(19) United States
(12) Patent Application Publication
(10) Pub. No.: US 2024/0277796 A1
(43) Pub. Date: Aug. 22, 2024

(54) METHODS FOR INHIBITING RAS

(71) Applicant: Revolution Medicines, Inc., Redwood City, CA (US)

(72) Inventor: Kyle SEAMON, San Mateo County, CA (US)

(21) Appl. No.: 18/518,027

(22) Filed: Nov. 22, 2023

Related U.S. Application Data
(63) Continuation of application No. PCT/US22/30823, filed on May 25, 2022.
(60) Provisional application No. 63/192,837, filed on May 25, 2021.

(51) Int. Cl. A61K 38/05; A61K 38/07; A61P 35/00

(52) U.S. Cl. CPC A61K 38/05; A61K 38/07; A61P 35/00

(57) ABSTRACT

The disclosure features methods for inhibiting RAS proteins. The disclosure also contains methods for the treatment of cancer.

### CROSS-REFERENCE TO RELATED APPLICATION

The present application is a continuation application of International Patent Application No. PCT/US2022/030823, filed on May 25, 2022, which claims the benefit of priority to U.S. Application No. 63/192,837, filed on May 25, 2021, which is hereby incorporated by reference in its entirety.

### BACKGROUND

Cancer remains one of the most-deadly threats to human health. In the U.S., cancer affects nearly 1.3 million new patients each year, and is the second leading cause of death after heart disease, accounting for approximately 1 in 4 deaths.

It has been well established in literature that RAS proteins (KRAS, HRAS, and NRAS) play an essential role in various human cancers and are therefore appropriate targets for anticancer therapy.

Indeed, mutations in RAS proteins account for approximately 30% of all human cancers in the United States, many of which are fatal. Dysregulation of RAS proteins by activating mutations, overexpression, or upstream activation is common in human tumors, and activating mutations in RAS are frequently found in human cancer. RAS converts between a GDP-bound “off” and a GTP-bound “on” state. The conversion between states is facilitated by interplay between a guanine nucleotide exchange factor (GEF) protein (e.g., SOS1), which loads RAS with GTP, and a GTPase-activating protein (GAP) protein (e.g., NF1), which hydrolyzes GTP, thereby inactivating RAS. Additionally, the SH2 domain-containing protein tyrosine phosphatase-2 (SHP2) associates with the receptor signaling apparatus and becomes active upon RTK activation, and then promotes RAS activation. Mutations in RAS proteins can lock the protein in the “on” state resulting in a constitutively active signaling pathway that leads to uncontrolled cell growth. For example, activating mutations at codon 12 in RAS proteins function by inhibiting both GAP-dependent and intrinsic hydrolysis rates of GTP, significantly skewing the population of RAS mutant proteins to the “on” (GTP-bound) state (RAS(ON)), leading to oncogenic MAPK signaling. Notably, RAS exhibits a picomolar affinity for GTP, enabling RAS to be activated even in the presence of low concentrations of this nucleotide. Mutations at codons 13 (e.g., G13D) and 61 (e.g., Q61K) of RAS are also responsible for oncogenic activity in some cancers.

First-in-class covalent inhibitors of the “off” form of RAS (RAS(OFF)) have demonstrated promising anti-tumor activity in cancer patients with oncogenic mutations in RAS. Further, therapeutic inhibition of the RAS pathway, although often initially efficacious, can ultimately prove ineffective as it may, for example, lead to over-activation of RAS pathway signaling via a number of mechanisms including, e.g., reactivation of the pathway via relief of the negative feedback machineries that naturally operate in these pathways, or may lead to resistance to RAS(OFF) inhibitors. Mutations contributing to resistance to such inhibitors have been reported (Tanaka et al., Clinical acquired resistance to KRASG12C inhibition through a novel KRAS switch-II pocket mutation and polyclonal alterations converging on RAS-MAPK reactivation, Cancer Discovery, Apr. 6, 2021. DOI: 10.1158/2159-8290. CD-21-0365; Awad et al., Mechanisms of acquired resistance to KRAS^{G12C} inhibition in cancer, AACR Annual Meeting 2021, Apr. 10, 2021). As a result, cells that were initially sensitive to such inhibitors may become resistant. Thus, a need exists for methods of effectively inhibiting RAS pathway signaling in cancer patients for whom RAS(OFF) inhibitors are not or may not be successful, including patients naïve to RAS(OFF) therapy.

### SUMMARY

The present disclosure provides methods for inhibiting RAS and for the treatment of cancer. The inventors observed that cancer cells treated with a RAS(OFF) inhibitor may develop resistance, e.g., through the acquisition of one or more mutations that render the RAS(OFF) inhibitor less effective or ineffective. The disclosure is based, at least in part, on the observation that some cancers resistant to treatment with a RAS(OFF) inhibitor remain responsive to treatment with a RAS(ON) inhibitor. Thus, administering a RAS(ON) inhibitor to a subject having cancer can slow or halt oncogenic signaling or disease progression where the cancer is resistant to treatment with a RAS(OFF) inhibitor. Additionally, administration of a RAS(ON) inhibitor, e.g., administered in combination with a RAS(OFF) inhibitor, may prevent the acquisition of one or more mutations in RAS that confer resistance to the RAS(OFF) inhibitor.

In addition, compounds disclosed herein may provide a clinical benefit for patients naïve to RAS(OFF) therapy.

In any embodiment herein, a RAS(ON) inhibitor may be a tri-complex RAS(ON) inhibitor, as that term is defined herein.

It is specifically contemplated that any limitation discussed with respect to one embodiment of the disclosure may apply to any other embodiment of the disclosure. Furthermore, any compound or composition of the disclosure may be used in any method of the disclosure, and any method of the disclosure may be used to produce or to utilize any compound or composition of the disclosure.

### Numbered Embodiments

1. A method of treating cancer in a subject in need thereof, wherein the cancer comprises:
(a) a first RAS mutation that is G12C and a second RAS mutation at a position selected from the group consisting of Y96, H95, R68, G13 and Q61; or
(b) a first RAS mutation at position G12,
wherein the cancer is resistant to treatment with a RAS(OFF) inhibitor, the method comprising administering to the subject a RAS(ON) inhibitor.

2. The method of embodiment 1, wherein the cancer does not comprise a KRAS Y96D mutation.

3. The method of embodiment 1, wherein the cancer does not comprise any of the following mutations: KRAS G12D, KRAS G12V, KRAS G12C, KRAS G12R, KRAS G12A, KRAS G12S, KRAS G12F, KRAS G12L, HRAS G12S, HRAS G12D, HRAS G12C, HRAS G12V, HRAS G12A, HRAS G12N, HRAS G12R, NRAS G12D, NRAS G12S, NRAS G12C, NRAS G12V, NRAS G12A, or NRAS G12R, or any combination thereof.

4. The method of any one of embodiments 1-3, wherein the cancer does not comprise a KRAS mutation selected from the group consisting of G12C^{amp}, G12D, G12R, G12V, G12W, G13D, Q61H, R68S, H95D, H95Q, H95R and Y96C, or any combination thereof.

5. The method of any one of embodiments 1-4, further comprising administering to the subject a RAS(OFF) inhibitor.

6. The method of embodiment 5, wherein the RAS(ON) inhibitor and the RAS(OFF) inhibitor are administered simultaneously or sequentially.

7. The method of embodiment 5 or 6 wherein the RAS(ON) inhibitor and the RAS(OFF) inhibitor are administered as a single formulation or in separate formulations.

8. The method of embodiment 6, wherein:
the RAS(OFF) inhibitor is administered for a first period of time; and
the RAS(ON) inhibitor is administered for a second period of time,
wherein the first period of time and the second period of time do not overlap and the first period of time precedes the second period of time.

9. The method of embodiment 6, wherein:
the RAS(OFF) inhibitor is administered for a first period of time; and
the RAS(OFF) inhibitor and RAS(ON) inhibitor are administered for a second period of time,
wherein the first period of time and the second period of time do not overlap and the first period of time precedes the second period of time.

10. The method of any one of embodiments 5-9, wherein the subject's cancer progresses on the RAS(OFF) inhibitor.

11. The method of embodiment 1, wherein the cancer comprises a first RAS mutation that is G12C and a second RAS mutation at position Y96.

12. The method of embodiment 1 or embodiment 11, wherein the second RAS mutation is selected from the group consisting of Y96C, Y96D, Y96F, Y96H, Y96N and Y96S.

13. The method of embodiment 1 or embodiment 11, wherein the second RAS mutation is selected from the group consisting of Y96D, Y96F, Y96H, Y96N and Y96S.

14. The method of embodiment 1 or embodiment 11, wherein the second RAS mutation is selected from the group consisting of Y96C, Y96F, Y96H, Y96N and Y96S.

15. The method of embodiment 1 or embodiment 11, wherein the second RAS mutation is selected from the group consisting of Y96F, Y96H, Y96N and Y96S.

16. The method of embodiment 1, wherein the cancer comprises a first RAS mutation that is G12C and a second RAS mutation at position H95 or R68.

17. The method of embodiment 1 or embodiment 16, wherein the first RAS mutation is G12C and the second RAS mutation is at position H95.

18. The method of any one of embodiments 1, 16 or 17, wherein the second RAS mutation is selected from the group consisting of H95D, H95L, H95N, H95P, H95Q, H95R and H95Y.

19. The method of any one of embodiments 1, 16 or 17, wherein the second RAS mutation is selected from the group consisting of H95L, H95N, H95P and H95Y.

20. The method of embodiment 1 or embodiment 16, wherein the first RAS mutation is G12C and the second RAS mutation is at position R68.

21. The method of any one of embodiments 1, 16 or 20, wherein the second mutation is selected from the group consisting of R68G, R68K, R68M, R68S, R68T and R68W.

22. The method of any one of embodiments 1, 16 or 20, wherein the second mutation is selected from the group consisting of R68G, R68K, R68M, R68T and R68W.

23. The method of any one of embodiments 1-4 and 11-22, wherein the subject has been treated with a RAS(OFF) inhibitor.

24. A method of treating cancer in a subject in need thereof, wherein the cancer comprises an amino acid substitution at RAS Y96, H95, or R68, the method comprising administering to the subject a RAS(ON) inhibitor.

25. The method of embodiment 24, wherein the cancer comprises a first RAS mutation that is G12C and a second RAS mutation at position Y96.

26. The method of embodiment 24 or embodiment 25, wherein the cancer does not comprise a Y96D RAS mutation.

27. The method of embodiment 25 or embodiment 26, wherein the second RAS mutation is selected from the group consisting of Y96C, Y96D, Y96F, Y96H, Y96N and Y96S.

28. The method of embodiment 25 or embodiment 26, wherein the second RAS mutation is selected from the group consisting of Y96D, Y96F, Y96H, Y96N and Y96S.

29. The method of embodiment 25 or embodiment 26, wherein the second RAS mutation is selected from the group consisting of Y96C, Y96F, Y96H, Y96N and Y96S.

30. The method of embodiment 25 or embodiment 26, wherein the second RAS mutation is selected from the group consisting of Y96F, Y96H, Y96N and Y96S.

31. The method of embodiment 25, wherein the cancer comprises a first RAS mutation that is G12C and a second RAS mutation at position H95 or R68.

32. The method of embodiment 25 or embodiment 31, wherein the first RAS mutation is G12C and the second RAS mutation is at position H95.

33. The method of any one of embodiments 25, 31 or 32, wherein the second RAS mutation is selected from the group consisting of H95D, H95L, H95N, H95P, H95Q, H95R and H95Y.

34. The method of any one of embodiments 25, 31 or 32, wherein the second RAS mutation is selected from the group consisting of H95L, H95N, H95P and H95Y.

35. The method of embodiment 25 or embodiment 31, wherein the first RAS mutation is G12C and the second RAS mutation is at position R68.

36. The method of any one of embodiments 25, 31 or 35, wherein the second mutation is selected from the group consisting of R68G, R68K, R68M, R68S, R68T and R68W.

37. The method of any one of embodiments 25, 31 or 35, wherein the second mutation is selected from the group consisting of R68G, R68K, R68M, R68T and R68W.

38. The method of embodiment 1, wherein the second mutation is Q61H.

39. The method of embodiment 1, wherein the second mutation is G13D.

40. The method of any one of embodiments 27-39, wherein the subject has been treated with a RAS(OFF) inhibitor.

41. The method of any one of embodiments 27-40, wherein the cancer is resistant to treatment with a RAS(OFF) inhibitor.

42. The method of embodiment 40 or embodiment 41, wherein the subject's cancer progresses on the RAS(OFF) inhibitor.

43. The method of any one of embodiments 1-42, wherein any RAS mutation is a KRAS mutation.

44. The method of any one of embodiments 1-42, wherein any RAS mutation is a NRAS mutation.

45. The method of any one of embodiments 1-42, wherein any RAS mutation is an HRAS mutation.

46. A method of treating cancer in a subject in need thereof, wherein the cancer comprises a RAS mutation selected from the group consisting of G12H, G12I, G12K, G12M, G12N, G12P, G12Q, G12T, G12W and G12Y, or a combination thereof, the method comprising administering to the subject a RAS(ON) inhibitor.

47. The method of embodiment 46, wherein the cancer further comprises a G12C RAS mutation.

48. The method of embodiment 46 or 47, wherein the subject has been treated with a RAS(OFF) inhibitor.

49. The method of any one of embodiments 46-48, wherein the cancer is resistant to treatment with a RAS(OFF) inhibitor.

50. The method of embodiment 48 or embodiment 49, wherein the subject's cancer progresses on the RAS(OFF) inhibitor.

51. A method of inhibiting RAS in a cell, wherein the RAS comprises an amino acid substitution at Y96, H95 or R68, the method comprising contacting the cell with a RAS(ON) inhibitor.

52. A method of inhibiting RAS in a cell, wherein the RAS comprises an amino acid substitution at H95 or R68, the method comprising contacting the cell with a RAS(ON) inhibitor.

53. The method of embodiment 51 or embodiment 52, wherein the cell is in vitro.

54. The method of embodiment 51 or claim 52, wherein the cell is in vivo.

55. The method of any one of embodiments 1-54, wherein the RAS(ON) inhibitor is an inhibitor selective for RAS G12C, G13D, or G12D.

56. The method of any one of embodiments 1-54, wherein the RAS(ON) inhibitor is a RAS(ON)^{MULTI} inhibitor.

57. The method of any one of embodiments 1-56, wherein the RAS(ON) inhibitor is a tri-complex RAS(ON) inhibitor.

58. The method of any one of embodiments 1-57, wherein the RAS(ON) inhibitor is selected from a compound disclosed in WO 2020132597.

59. The method of any one of embodiments 1-57, wherein the RAS(ON) inhibitor is a compound of Formula AI:

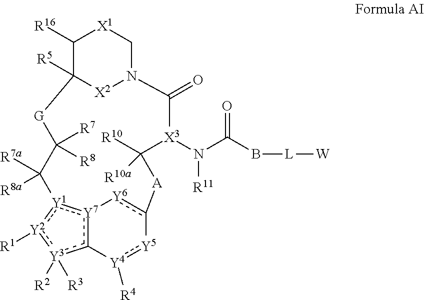

or a pharmaceutically acceptable salt thereof,
wherein the dotted lines represent zero, one, two, three, or four non-adjacent double bonds;
   A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
   B is absent, —CH(R⁹)—, or >C═CR⁹R9′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
   G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
   L is absent or a linker;
   W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
   X¹ is optionally substituted C₁-C₂ alkylene, NR, O, or S(O)ₙ;
   X² is O or NH;
   X³ is N or CH;
   n is 0, 1, or 2;
   R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
   each R′ is, independently, H or optionally substituted C₁-C₄ alkyl;
   Y¹ is C, CH, or N;
   Y², Y³, Y⁴, and Y⁷ are, independently, C or N;
   Y⁵ is CH, CH₂, or N;
   Y⁶ is C(O), CH, CH₂, or N;
   R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
   R¹ and R² combine with the atoms to which they are attached to form an optionally substituted 3 to 14-membered heterocycloalkyl;
   R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
   R³ is absent, or
   R² and R³ combine with the atom to which they are attached to form an optionally substituted 3 to 8-membered cycloalkyl or optionally substituted 3 to 14-membered heterocycloalkyl;
   R⁴ is absent, hydrogen, halogen, cyano, or methyl optionally substituted with 1 to 3 halogens;
   R⁵ is hydrogen, C₁-C₄ alkyl optionally substituted with halogen, cyano, hydroxy, or C₁-C₄ alkoxy, cyclopropyl, or cyclobutyl;
   R⁶ is hydrogen or methyl; R⁷ is hydrogen, halogen, or optionally substituted C₁-C₃ alkyl, or
   R⁶ and R⁷ combine with the carbon atoms to which they are attached to form an optionally substituted 3 to 6-membered cycloalkyl or optionally substituted 3 to 7-membered heterocycloalkyl;
   R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
   R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
   R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
   R⁷′ and R⁸′ combine with the carbon atom to which they are attached to form optionally substituted 3 to 6-membered cycloalkyl or optionally substituted 3 to 7-membered heterocycloalkyl;
   R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
   R⁹ and L combine with the atoms to which they are attached to form an optionally substituted 3 to 14-membered heterocycloalkyl;
   R⁹′ is hydrogen or optionally substituted C₁-C₆ alkyl;
   R¹⁰ is hydrogen, halo, hydroxy, C₁-C₃ alkoxy, or C₁-C₃ alkyl;
   R^{10a} is hydrogen or halo;
   R¹¹ is hydrogen or C₁-C₃ alkyl;
   R¹⁶ is hydrogen or C₁-C₃ alkyl.

60. The method of any one of embodiments 1-57, wherein the RAS(ON) inhibitor is selected from a compound of Table A1 or Table A2, or a pharmaceutically acceptable salt thereof.

61. The method of any one of embodiments 1-57, wherein the RAS(ON) inhibitor is a compound of Formula BI:

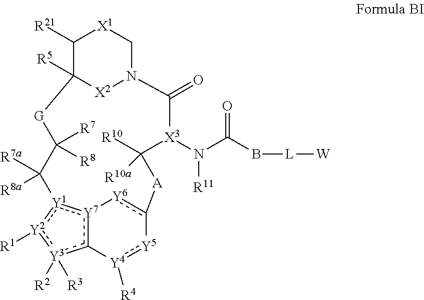

or a pharmaceutically acceptable salt thereof,
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R⁹′, or >CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl.

62. The method of any one of embodiments 1-57, wherein the RAS(ON) inhibitor is selected from a compound of Table B1 or Table B2, or a pharmaceutically acceptable salt thereof.

63. The method of any one of embodiments 1-57, wherein the RAS(ON) inhibitor is a compound of Formula CI, or a pharmaceutically acceptable salt thereof.

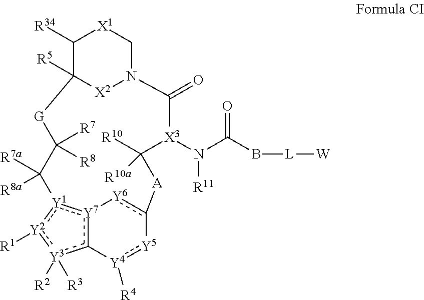

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl; and
R³⁴ is hydrogen or C₁-C₃ alkyl.

64. The method of any one of embodiments 1-57, wherein the RAS(ON) inhibitor is selected from a compound of Table C1 or Table C₂, or a pharmaceutically acceptable salt thereof.

65. The method of any one of embodiments 1-57, wherein the RAS(ON) inhibitor is a compound of Formula DIa:

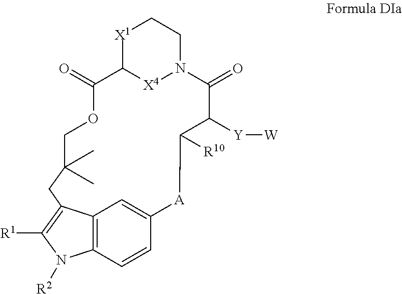

or a pharmaceutically acceptable salt thereof,
wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;

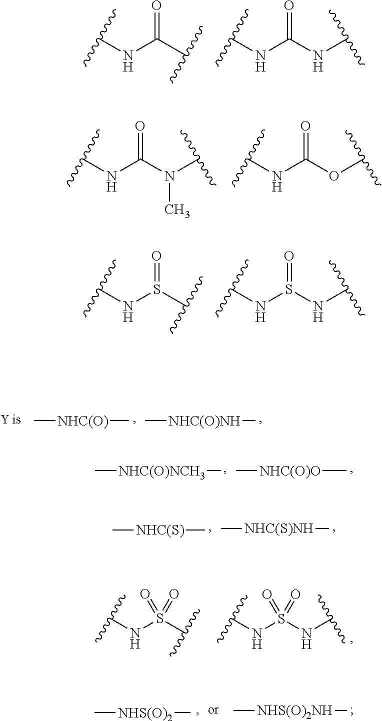

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R¹⁰ is hydrogen, hydroxy, optionally substituted C₁-C₃ alkyl, or optionally substituted C₁-C₆ heteroalkyl.

66. The method of any one of embodiments 1-57, wherein the RAS(ON) inhibitor is selected from a compound of Table D1a or D1 b, or a pharmaceutically acceptable salt thereof.

67. The method of any one of embodiments 1-57, wherein the RAS(ON) inhibitor is a compound of Formula EI:

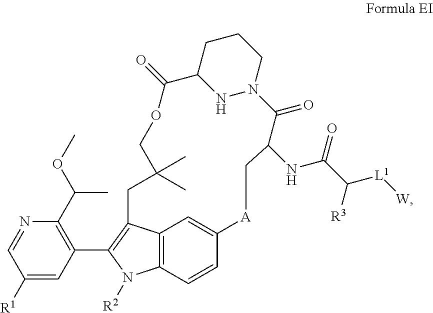

or a pharmaceutically acceptable salt thereof,
wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
L¹ is absent or a linker;
W is a cross-linking group comprising a vinyl ketone, vinyl sulfone, ynone, or an alkynyl sulfone;
R¹ is hydrogen, optionally substituted 3 to 10-membered heterocycloalkyl, or optionally substituted C₁-C₆ heteroalkyl;
R² is optionally substituted C₁-C₆ alkyl; and
R³ is optionally substituted C₁-C₆ alkyl or optionally substituted C₁-C₃ heteroalkyl.

68. The method of any one of embodiments 1-57, wherein the RAS(ON) inhibitor is selected from a compound of Table E1, or a pharmaceutically acceptable salt thereof.

69. The method of any one of embodiments 1-57, wherein the RAS(ON) inhibitor is a compound of Formula FI:

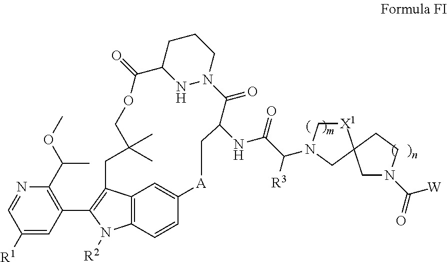

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
X¹ is CH₂ or O;
m is 1 or 2;
n is 0 or 1;
R¹ is hydrogen or optionally substituted 3 to 10-membered heterocycloalkyl;
R² is optionally substituted C₁-C₆ alkyl; and
R³ is optionally substituted C₁-C₆ alkyl or optionally substituted 3 to 6-membered cycloalkyl.

70. The method of any one of embodiments 1-57, wherein the RAS(ON) inhibitor is selected from a compound of Table F1, Table F2, Table F3, Table F4, Table F5, or Table F6.

71. The method of any one of embodiments 1-23, 40-45, or 48-50, wherein the RAS(OFF) inhibitor selectively targets RAS G12C.

72. The method of any one of embodiments 1-23, 40-45, or 48-50, wherein the RAS(OFF) inhibitor is selected from sotorasib (AMG 510), adagrasib (MRTX849), MRTX1257, JNJ-74699157 (ARS-3248), LY3537982, LY3499446, ARS-853, ARS-1620, GDC-6036, JDQ443, BPI-421286, JAB-21000, RSC-1255, ERAS-3490, D-1553, JAB-21822, GH-35, ICP-915, IBI351, and B11823911.

73. The method of any one of embodiments 1-72, wherein the cancer is selected from colorectal cancer, non-small cell lung cancer, small-cell lung cancer, pancreatic cancer, appendiceal cancer, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, bladder cancer and melanoma.

74. The method of embodiment 76, wherein the cancer is non-small cell lung cancer.

75. The method of any one of embodiments 1-74, wherein the method further comprises administering to the subject or the cell an additional anti-cancer therapy.

### BRIEF DESCRIPTION OF THE FIGURES

FIG. 1A and FIG. 1B. Compound AA, a tri-complex KRAS^{G12C}(ON) inhibitor disclosed herein as a compound of Formula B1 herein, and also a compound of Table B1 herein, and also found in WO 2021/091982, is active against second site mutations conferring resistance to KRAS^{G12C}(OFF) inhibitors MRTX849 and AMG 510. FIG. 1A is a heatmap representing cellular RAS/RAF disruption assay results regarding various KRAS mutations in the presence of different RAS inhibitors. Certain mutations have been observed in patients treated with AMG 510 (e.g., Y96C, Y96D, H95D, H95Q, H95R, R68S) (Tanaka et al., Clinical acquired resistance to KRASG12C inhibition through a novel KRAS switch-II pocket mutation and polyclonal alterations converging on RAS-MAPK reactivation, Cancer Discovery, Apr. 6, 2021. DOI: 10.1158/2159-8290. CD-21-0365; Awad et al., Mechanisms of acquired resistance to KRAS^{G12C} inhibition in cancer, AACR Annual Meeting 2021, Apr. 10, 2021). FIG. 1B shows the IC50 value associated with each colored bar of the heatmap. See Example 1.

FIG. 2A and FIG. 2B. Compound A, a tri-complex KRAS^{MULTI}(ON) inhibitor disclosed herein as a compound of Formula D1 herein, and also a compound of Table D1 herein, and also found in WO 2022/060836, is active against RAS oncogene switching mutations observed in KRAS^{G12C}(OFF) resistance. FIG. 2A is a heatmap representing cellular RAS/RAF disruption assay results regarding various KRAS mutations in the presence of different RAS inhibitors. Certain mutations have been observed in patients treated with AMG 510 (e.g., G12C, G12F, G12R, G12V, G12W) (Tanaka et al.; Awad et al.). FIG. 2B shows the IC50 value associated with each colored bar of the heatmap. See Example 2.

FIG. 3 demonstrates in vitro efficacy of Compound A, a tri-complex KRAS^{MULTI}(ON) inhibitor disclosed herein, in multiple RAS-driven cancer cell lines. Each graph shows cell proliferation (% relative to control) vs. log M [Compound A]. Potency of in vitro cell proliferation inhibition of Capan-1 (KRAS^{G12V}) AsPC-1 (KRAS^{G12D}), HCT116 (KRAS^{G13D}), SK-MEL-30 (NRAS^{Q61K}), NCI-H1975 (EGFR^{T790M/L858R}) and A375 (BRAF^{V600E}) cells exposed to Compound A for 120 hours. Data represent the mean of multiple experiments. See Example 3.

### DETAILED DESCRIPTION

The present disclosure relates generally to methods for inhibiting RAS and for the treatment of cancer. In some embodiments, the disclosure provides methods for delaying, preventing, or treating acquired resistance to a RAS(OFF) inhibitor by administering a RAS(ON) inhibitor. In some embodiments, administration of a RAS(ON) inhibitor, e.g., administered in combination with a RAS(OFF) inhibitor, may prevent the acquisition of one or more mutations in RAS that confers resistance to the RAS(OFF) inhibitor. In addition, compounds disclosed herein may provide a clinical benefit for patients naïve to RAS(OFF) therapy.

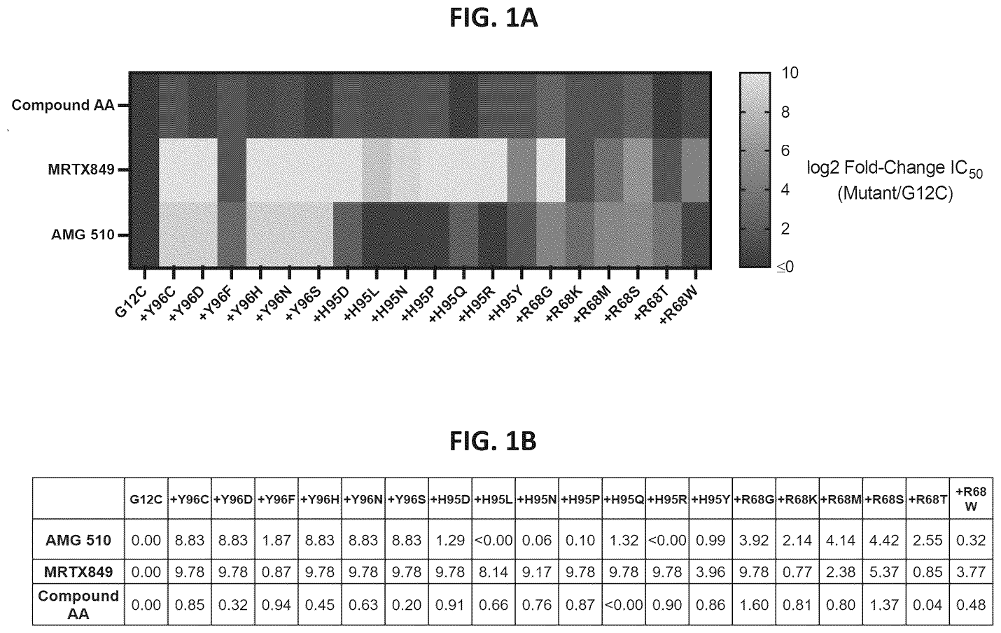

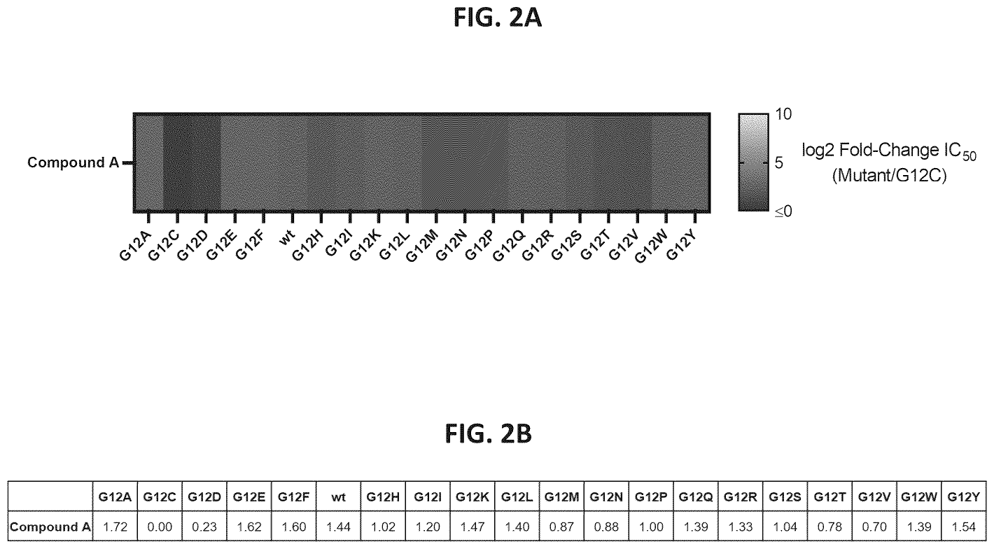

The heatmaps shown in FIG. 1A and FIG. 2A represent relative potencies observed in cellular assays measuring the abundance of protein complexes between the active form of RAS, RAS(ON), and its signaling partner, RAF kinase. Each tri-complex KRAS(ON) inhibitor maximally disrupted KRAS^{G12C}(ON)/CRAF complexes (data not shown), indicative of blockade of KRAS^{G12C} activation of RAF and the MAPK cascade.

Recently, two groups published the first descriptions of genetic mutations observed in ctDNA samples from patients who had exhibited resistance to adagrasib therapy (MRTX849, a KRAS^{G12C}(OFF) inhibitor in clinical development). Tanaka et al., Clinical acquired resistance to KRAS^{G12C} inhibition through a novel KRAS switch-II pocket mutation and polyclonal alterations converging on RAS-MAPK reactivation, Cancer Discovery, Apr. 6, 2021. DOI: 10.1158/2159-8290. CD-21-0365; Awad et al., Mechanisms of acquired resistance to KRAS^{G12C} inhibition in cancer, AACR Annual Meeting 2021, Apr. 10, 2021. Some of these mutations were studied herein, as described below.

One set of mutations (FIG. 1A, FIG. 1B) convey second site mutations in KRAS^{G12C} occurring on the same allele as the G12C mutation (in cis). These mutations confer resistance to KRAS^{G12C}(OFF) inhibitors via alteration of the binding site of that inhibitor class. This resistance is clearly represented in the heatmap (FIG. 1A) which reflects fold change in inhibitor IC50 for the indicated double mutant relative to the single G12C mutant—with yellow representing the largest fold change. For the KRAS^{G12C}(OFF) inhibitors MRTX849 and AMG 510, there is a decrease in potency (i.e., increase in fold change) for the majority of the double mutants relative to the single G12C mutant (all second site mutations are measured in cis with G12C). Compound AA, a tri-complex KRAS^{G12C}(ON) inhibitor disclosed herein, is active against all of the second site mutations tested with minimal fold change in potency relative to the single G12C mutant, indicating these mutations are not sufficient to confer resistance to Compound AA, or more broadly, as the inventors surmise, tri-complex G12C(ON) inhibitors generally (see, e.g., Tanaka et al.) and also that Compound AA and other tri-complex RAS(ON) inhibitors disclosed herein may offer clinical benefit in treating patients who are not only resistant to (e.g., have progressed on) KRAS^{G12C}(OFF) inhibitors, but patients naïve to such treatment whose tumors bear one or more of these second site KRAS mutations, as well as comparable positions in HRAS and NRAS.

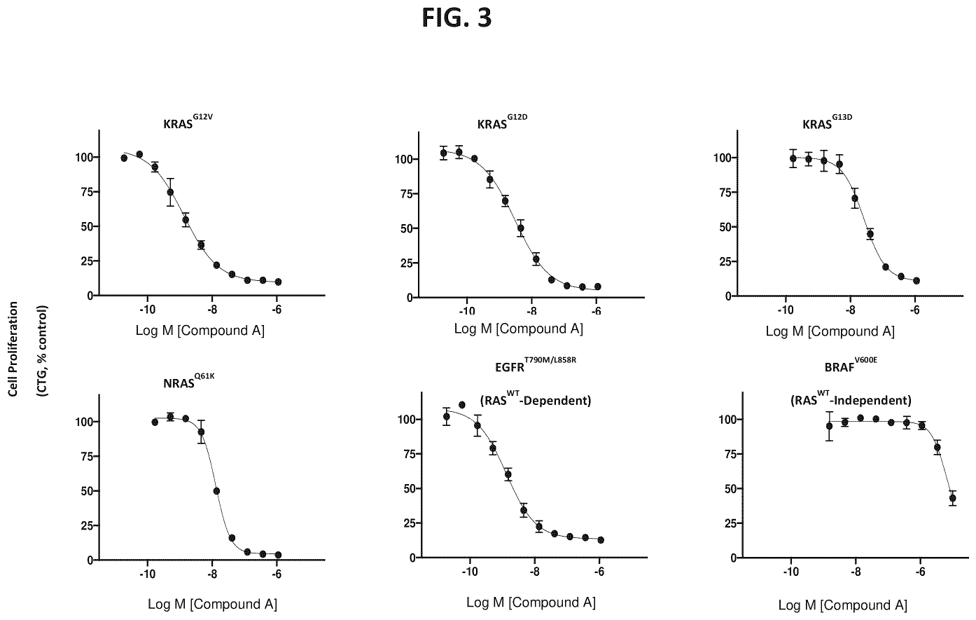

The second set of mutations (FIG. 2A, FIG. 2B) are alternative oncogenic RAS mutations. The inventors have previously disclosed cellular data demonstrating the ability of a tri-complex KRAS^{MULTI}(ON) inhibitor to inhibit the proliferation of cancer cells bearing a range of oncogenic RAS mutants (FIG. 3). The heatmap (FIG. 2A) demonstrates comprehensively that Compound A, a tri-complex KRAS^{MULTI}(ON) inhibitor disclosed herein, can inhibit KRAS^{G12X}/RAF complex formation and therefore signaling driven by all possible G12 mutants of KRAS. These data indicate that Compound A, or more broadly, as the inventors surmise, tri-complex RAS(ON) inhibitors generally (see, e.g., Tanaka et al.), may offer clinical benefit in treating not only patients who are resistant to (e.g., have progressed on) KRAS^{G12C}(OFF) inhibitors, but patients naïve to such treatment whose tumors bear one or more of these alternative KRAS mutations, as well as comparable 12 position in HRAS and NRAS.

### General Methods

The practice of the present disclosure will employ, unless otherwise indicated, conventional techniques of cell culturing, molecular biology (including recombinant techniques), microbiology, cell biology, biochemistry, and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present disclosure include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present disclosure described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{o}; —(CH₂)₀₋₄OR^{o}; —O(CH₂)₀₋₄R^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄—C(O)—N(R^{o})₂; —(CH₂)₀₋₄—C(O)—N(R^{o})—S(O)₂—R^{o}; —C(NCN)NR^{o}₂; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}; —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O) NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NOR^{o})NR^{o}₂; —C(NH)NR^{o}₂; —P(O)₂R^{o}; —P(O)R^{o}₂; —P(O)(OR^{o})₂; —OP(O)R^{o}₂; —OP(O)(OR^{o})₂; —OP(O)(OR^{o})R^{o}, —SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, —C₁₋₆ aliphatic, —CH₂Ph, —O(CH₂)₀₋₁ Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), may be, independently, halogen, —(CH₂)₀₋₂R^{○}, -(haloR^{○}), —(CH₂)₀₋₂OH, —(CH₂)₀₋₂OR^{○}, —(CH₂)₀₋₂CH(OR^{○})₂; —O(haloR^{○}), —CN, —N₃, —(CH₂)₀₋₂C(O)R^{○}, —(CH₂)₀₋₂C(O)OH, —(CH₂)₀₋₂C(O)OR^{○}, —(CH₂)₀₋₂SR^{○}, —(CH₂)₀₋₂SH, —(CH₂)₀₋₂NH₂, —(CH₂)₀₋₂NHR^{○}, —(CH₂)_{0- 2}NR^{○}₂, —NO₂, —SiR^{○}₃, —OSiR^{○}₃, —C(O)SR^{○}, —(C₁₋₄ straight or branched alkylene)C(O)OR^{○}, or —SSR^{○} wherein each R^{○} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{○} include halogen, —R^{○}, -(haloR^{○}), —OH, —OR^{○}, —O(haloR^{○}), —CN, —C(O)OH, —C(O)OR^{○}, —NH₂, —NHR^{○}, —NR^{○}₂, or —NO₂, wherein each R^{○} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{○}, -(haloR^{○}), —OH, —OR^{○}, —O(haloR^{○}), —CN, —C(O)OH, —C(O)OR^{○}, —NH₂, —NHR^{○}, —NR^{○}₂, or —NO₂, wherein each R^{○} is unsubstituted or where preceded by “halo” is substituted only with one or more halogens, and is independently C₁₋₄ aliphatic, —CH₂Ph, —O(CH₂)₀₋₁ Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

As used herein, the term “administration” refers to the administration of a composition (e.g., a compound, or a preparation that includes a compound as described herein) to a subject or system. Administration also includes administering a prodrug derivative or analog of the compound or pharmaceutically acceptable salt of the compound or composition to the subject, which can form an equivalent amount of active compound within the subject's body. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “CₓC_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

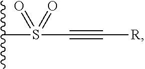

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

An “amino acid substitution,” as used herein, refers to the substitution of a wild-type amino acid of a protein with a non-wild-type amino acid. Amino acid substitutions can result from genetic mutations and may alter one or more properties of the protein (e.g., may confer altered binding affinity or specificity, altered enzymatic activity, altered structure, or altered function). For example, where a RAS protein includes an amino acid substitution at position Y96, this notation indicates that the wild-type amino acid at position 96 of the RAS protein is a Tyrosine (Y), and that the RAS protein including the amino acid substitution at position Y96 includes any amino acid other than Tyrosine (Y) at position 96. The notation Y96D indicates that the wild-type Tyrosine (Y) residue at position 96 has been substituted with an Aspartic Acid (D) residue.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “combination therapy” refers to a method of treatment including administering to a subject at least two therapeutic agents, optionally as one or more pharmaceutical compositions, as part of a therapeutic regimen. For example, a combination therapy may include administration of a single pharmaceutical composition including at least two therapeutic agents and one or more pharmaceutically acceptable carrier, excipient, diluent, or surfactant. A combination therapy may include administration of two or more pharmaceutical compositions, each composition including one or more therapeutic agent and one or more pharmaceutically acceptable carrier, excipient, diluent, or surfactant. In various embodiments, at least one of the therapeutic agents is a RAS(ON) inhibitor (e.g., any one or more KRAS(ON) inhibitors disclosed herein or known in the art). In various embodiments, at least one of the therapeutic agents is a RAS(OFF) inhibitor (e.g., any one or more KRAS(OFF) inhibitors disclosed herein or known in the art). The two or more agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due to an additive or synergistic effect of combining the two or more therapeutics.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

As used herein, the term “dosage form” refers to a physically discrete unit of a compound (e.g., a compound of the present disclosure) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present disclosure) has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The term “disorder” is used in this disclosure to mean, and is used interchangeably with, the terms disease, condition, or illness, unless otherwise indicated.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

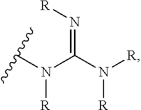

wherein each R is, independently, any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein. In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. Small molecule inhibitors include cyclic and acyclic compounds. Small molecules inhibitors include natural products, derivatives, and analogs thereof. Small molecule inhibitors can include a covalent cross-linking group capable of forming a covalent cross-link, e.g., with an amino acid side-chain of a target protein.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting a first moiety (e.g., a macrocyclic moiety or B) to a second moiety (e.g., W) in a compound of any one of Formula AI, Formula BI, Formula CI, Formula DIA, Formula EI, Formula FI, Formula FIII, or a subformula thereof, such that the resulting compound is capable of achieving an IC50 of 2 uM or less in the Ras-RAF disruption assay protocol provided here:
The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless Cyclophilin A, His 6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu-W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras:RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “mutation” as used herein indicates any modification of a nucleic acid or polypeptide which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications or chromosomal breaks or translocations. In particular embodiments, the mutation results in an amino acid substitution in the encoded-protein.

A “patient” or “subject” is a mammal, e.g., a human, mouse, rat, guinea pig, dog, cat, horse, cow, pig, or non-human primate, such as a monkey, chimpanzee, baboon or rhesus.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The term “preventing acquired resistance,” as used herein, means avoiding the occurrence of acquired or adaptive resistance. For example, the use of a RAS(ON) inhibitor described herein in preventing acquired/adaptive resistance to a RAS(OFF) inhibitor means that the RAS(ON) inhibitor is administered prior to any detectable existence of resistance to the RAS(OFF) inhibitor and the result of such administration of the RAS(ON) inhibitor is that no resistance to the RAS(OFF) inhibitor occurs.

As used herein, the term “pharmaceutical composition” refers to a compound, such as a compound of the present disclosure, or a pharmaceutically acceptable salt thereof, formulated together with a pharmaceutically acceptable excipient.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The terms “RAS inhibitor” and “inhibitor of [a] RAS” are used interchangeably to refer to any inhibitor that targets, that is, selectively binds to or inhibits a RAS protein. In various embodiments, these terms include RAS(OFF) and RAS(ON) inhibitors.

As used herein, the term “RAS(ON) inhibitor” refers to an inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). RAS(ON) inhibitors described herein include compounds of Formula AI, Formula BI, Formula CI, Formula DIa, Formula EI, Formula FI, Formula FIII, and subformulas thereof, and compounds of Table A1, Table A2, Table B1, Table B2, Table C1, Table C₂, Table D1a, Table D1b, Table D2, Table D3, Table E1, Table F1, Table F2, Table F3, Table F4, Table F5, Table F6, as well as salts (e.g., pharmaceutically acceptable salts), solvates, hydrates, stereoisomers (including atropisomers), and tautomers thereof. In some embodiments, a RAS(ON) inhibitor is a tri-complex RAS(ON) inhibitor, as that term is defined herein.

As used herein, the term “RAS(OFF) inhibitor” refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS(OFF) inhibitors may also bind to or inhibit the GTP-bound, active state of RAS (e.g., with a lower affinity or inhibition constant than for the GDP-bound, inactive state of RAS).

As used herein, the term “RAS^{MULTI}(ON) inhibitor” refers to a RAS(ON) inhibitor of at least 3 RAS variants with missense mutations at one of the following positions: 12, 13, 59, 61, or 146. In some embodiments, a RAS^{MULTI}(ON) inhibitor refers to a RAS^{MULTI}(ON) inhibitor of at least 3 RAS variants with missense mutations at one of the following positions: 12, 13, and 61. In some embodiments, a RAS^{MULTI}(ON) inhibitor is a tri-complex RAS^{MULTI}(ON) inhibitor.

The terms “RAS pathway” and “RAS/MAPK pathway” are used interchangeably herein to refer to a signal transduction cascade downstream of various cell surface growth factor receptors in which activation of RAS (and its various isoforms and alleotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

As used herein, the term “resistant to treatment” refers to a treatment of a disorder with a therapeutic agent, where the therapeutic agent is ineffective or where the therapeutic agent was previously effective and has become less effective over time. Resistance to treatment includes acquired resistance to treatment, which refers to a decrease in the efficacy of a treatment over a period of time where the subject is being administered the therapeutic agent. Acquired resistance to treatment may result from the acquisition of a mutation in a target protein that renders the treatment ineffective or less effective. Accordingly, resistance to treatment may persist even after cessation of administration of the therapeutic agent. In particular, a cancer may become resistant to treatment with a RAS(OFF) inhibitor by the acquisition of a mutation (e.g., in the RAS protein) that decreases the efficacy of the RAS(OFF) inhibitor. Measurement of a decrease in the efficacy of the treatment will depend on the disorder being treated, and such methods are known to those of skill in the art. For example, efficacy of a cancer treatment may be measured by the progression of the disease. An effective treatment may slow or halt the progression of the disease. A cancer that is resistant to treatment with a therapeutic agent, e.g., a RAS(OFF) inhibitor, may fail to slow or halt the progression of the disease.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

A “therapeutic agent” is any substance, e.g., a compound or composition, capable of treating a disease or disorder. In some embodiments, therapeutic agents that are useful in connection with the present disclosure include RAS inhibitors and cancer chemotherapeutics. Many such therapeutic agents are known in the art and are disclosed herein.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

A “therapeutic regimen” refers to a dosing regimen whose administration across a relevant population is correlated with a desired or beneficial therapeutic outcome.

The term “thiocarbonyl,” as used herein, refers to a —C(S)— group. The term “treatment” (also “treat” or “treating”), in its broadest sense, refers to any administration of a substance (e.g., a compound of the present disclosure) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “treatment” (also “treat” or “treating”), in its broadest sense, refers to any administration of a substance (e.g., a compound of the present disclosure) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond. The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

The term “ynone,” as used herein, refers to a group comprising the structure

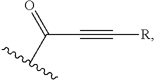

wherein R is any chemically feasible substituent described herein.

### RAS Inhibitors

Provided herein are compounds that inhibit RAS and uses thereof. Also provided are pharmaceutical compositions including one or more RAS inhibitor compounds, or a pharmaceutically acceptable salt thereof, and a pharmaceutically acceptable excipient. RAS inhibitor compounds may be used in methods of inhibiting RAS (e.g., in a subject or in a cell) and methods of treating cancer, as described herein. In some embodiments, a compound of the present disclosure is or acts as a prodrug, such as with respect to administration to a cell or to a subject in need thereof.

### RAS(ON) Inhibitors

Provided herein are RAS(ON) inhibitors. A RAS(ON) inhibitor targets, that is, selectively binds to or inhibits the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS).

In some embodiments, the RAS(ON) inhibitor is selected from a tri-complex inhibitor disclosed in WO 202132597, WO 2021091956, WO 2021091982, or WO 2021091967, or a compound disclosed in Table A1, Table A2, Table B1, Table B2, Table C1, Table C₂, Table D1a, Table D1 b, Table D2, Table D3, Table E1, Table F1, Table F2, Table F3, Table F4, Table F5, Table F6, or a compound of Formula AI, Formula BI, Formula CI, Formula DIa, Formula EI, Formula FI, Formula FIII, and subformulas thereof, o. In some embodiments, the RAS(ON) inhibitor is a compound described by a Formula in WO 2020132597, such as a compound of FIG. 1 therein, or a pharmaceutically acceptable salt thereof.

In some embodiments, the RAS(ON) inhibitor is selective for RAS that includes an amino acid substitution at G12, G13, Q61, or a combination thereof. In some embodiments, the RAS(ON) inhibitor is selective for RAS that includes an amino acid substitution selected from G12C, G12D, G12V, G13C, G13D, Q61 L, or a combination thereof. In some embodiments, the RAS(ON) inhibitor is selective for RAS that includes a G12C amino acid substitution.

In some embodiments, the RAS(ON) inhibitor is a KRAS(ON) inhibitor, where a KRAS(ON) inhibitor refers to an inhibitor that targets, that is, selectively binds to or inhibits the GTP-bound, active state of KRAS (e.g., selective over the GDP-bound, inactive state of KRAS). In some embodiments, the KRAS(ON) inhibitor is selective for KRAS that includes an amino acid substitution at G12, G13, Q61, A146, K117, L19, Q22, V14, A59, or a combination thereof. In some embodiments, the KRAS(ON) inhibitor is selective for KRAS that includes an amino acid substitution selected from G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, G13V, or a combination thereof.

In some embodiments, the RAS(ON) inhibitor is an NRAS(ON) inhibitor, where an NRAS(ON) inhibitor refers to an inhibitor that targets, that is, selectively binds to or inhibits the GTP-bound, active state of NRAS (e.g., selective over the GDP-bound, inactive state of NRAS). In some embodiments, the NRAS(ON) inhibitor is selective for NRAS that includes an amino acid substitution at G12, G13, Q61, P185, A146, G60, A59, E132, E49, T50, or a combination thereof. In some embodiments, the NRAS(ON) inhibitor is selective for NRAS that includes an amino acid substitution selected from Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, A59T, or a combination thereof.

In some embodiments, the RAS(ON) inhibitor is an HRAS(ON) inhibitor, where an HRAS(ON) inhibitor refers to an inhibitor that targets, that is selectively binds to or inhibits the GTP-bound, active state of HRAS (e.g., selective over the GDP-bound, inactive state of HRAS). In some embodiments, the HRAS(ON) inhibitor is selective for HRAS that includes an amino acid substitution at G12, G13, Q61, K117, A59, A18, D119, A66, A146, or a combination thereof. In some embodiments, the HRAS(ON) inhibitor is selective for NRAS that includes an amino acid substitution selected from Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, G12R, or a combination thereof.

In some embodiments, the RAS(ON) inhibitor is a RAS(ON)^{MULTI} inhibitor.

In some embodiments, a RAS(ON) inhibitor described herein entails formation of a high affinity three-component complex (“tri complex”) between a synthetic ligand and two intracellular proteins which do not interact under normal physiological conditions: the target protein of interest (e.g., RAS), and a widely expressed cytosolic chaperone (presenter protein) in the cell (e.g., cyclophilin A). More specifically, in some embodiments, the RAS(ON) inhibitors described herein induce a new binding pocket in RAS by driving formation of a high affinity tri-complex between the RAS protein and the widely expressed cytosolic chaperone, cyclophilin A (CYPA). Without being bound by theory, one way the inhibitory effect on Ras is affected by compounds of the invention and the complexes they form is by steric occlusion of the interaction site between Ras and downstream effector molecules, such as RAF and PI3K, which are required for propagating the oncogenic signal. In some embodiments, a RAS(ON) inhibitor is a tri-complex RAS^{G12C}(ON) inhibitor. In some embodiments, a RAS(ON) inhibitor is a tri-complex RAS^{G12D}(ON) inhibitor. In some embodiments, a RAS(ON) inhibitor is a tri-complex RAS^{MULTI}(ON) inhibitor. Such tri-complex RAS(ON) inhibitors may inhibit KRAS, HRAS or NRAS, or a combination thereof.

In some embodiments, the RAS(ON) inhibitor is a compound, or pharmaceutically acceptable salt thereof, having the structure of Formula A00:

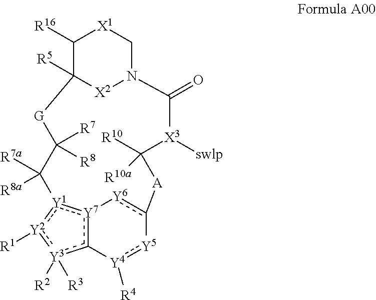

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
swIp (Switch I/P-loop) refers to an organic moiety that non-covalently binds to both the Switch I binding pocket and residues 12 or 13 of the P-loop of a Ras protein (see, e.g., Johnson et al., 292:12981-12993 (2017), incorporated herein by reference);
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R¹⁰ is hydrogen, halo, hydroxy, C₁-C₃ alkoxy, or C₁-C₃ alkyl;
R^{10a} is hydrogen or halo; and
R¹⁶ is hydrogen or C₁-C₃ alkyl (e.g., methyl). In some embodiments, the resulting compound is capable of achieving an IC50 of 2 uM or less (e.g., 1.5 uM, 1 uM, 500 nM, or 100 nM or less) in the Ras-RAF disruption assay protocol described herein.

In some embodiments, the disclosure features a compound, or pharmaceutically acceptable salt thereof, of structural Formula AI:

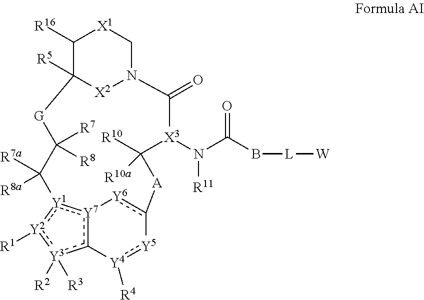

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂; each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R¹⁶ is hydrogen or C₁-C₃ alkyl (e.g., methyl).

In some embodiments, the disclosure features a compound, or pharmaceutically acceptable salt thereof, of structural Formula Ala:

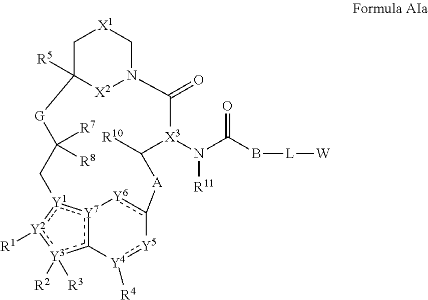

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂; each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the disclosure features a compound, or pharmaceutically acceptable salt thereof, of structural Formula AIb:

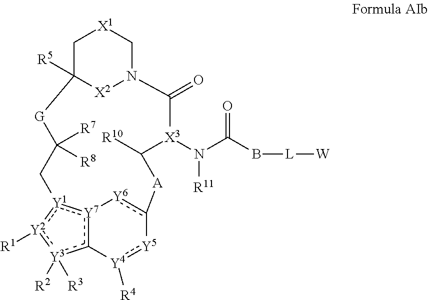

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula AI and subformula thereof, G is optionally substituted C₁-C₄ heteroalkylene.

In some embodiments, the RAS(ON) inhibitor has the structure of Formula AIc, or a pharmaceutically acceptable salt thereof:

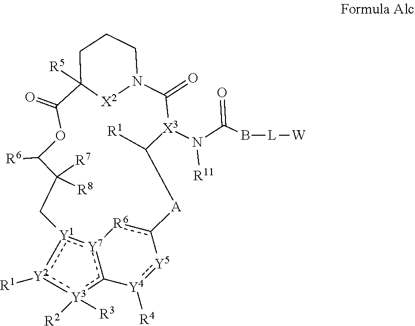

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula AI and subformula thereof, X² is NH. In some embodiments of Formula AI and subformula thereof, X³ is CH.

In some embodiments of Formula AI and subformula thereof, R¹¹ is hydrogen. In some embodiments of Formula AI and subformula thereof, R¹¹ is C₁-C₃ alkyl. In some embodiments of Formula AI and subformula thereof, R¹¹ is methyl.

In some embodiments, the RAS(ON) inhibitor has the structure of Formula AId, or a pharmaceutically acceptable salt thereof:

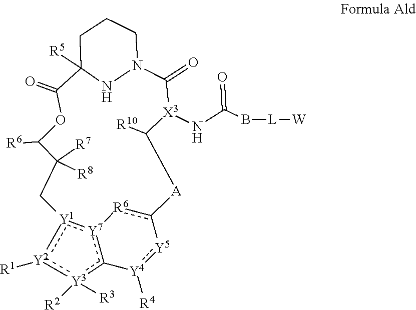

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of compounds of the present invention, X¹ is optionally substituted C₁-C₂ alkylene. In some embodiments, X¹ is methylene. In some embodiments, X¹ is methylene substituted with a C₁-C₆ alkyl group or a halogen. In some embodiments, X¹ is —CH(Br)—. In some embodiments, X¹ is —CH(CH₃)—.

In some embodiments of Formula AI and subformula thereof, R³ is absent.

In some embodiments of Formula AI and subformula thereof, R⁴ is hydrogen.

In some embodiments of Formula AI and subformula thereof, R⁵ is hydrogen. In some embodiments of Formula AI and subformula thereof, R⁵ is C₁-C₄ alkyl optionally substituted with halogen.

In some embodiments of Formula AI and subformula thereof, R⁵ is methyl.

In some embodiments of Formula AI and subformula thereof, Y⁴ is C. In some embodiments of Formula AI and subformula thereof, Y⁵ is CH. In some embodiments of Formula AI and subformula thereof, Y⁶ is CH. In some embodiments of Formula AI and subformula thereof, Y¹ is C. In some embodiments of Formula AI and subformula thereof, Y² is C. In some embodiments of Formula AI and subformula thereof, Y³ is N. In some embodiments of Formula AI and subformula thereof, Y⁷ is C.

In some embodiments, the RAS(ON) inhibitor has the structure of Formula Ale, or a pharmaceutically acceptable salt thereof:

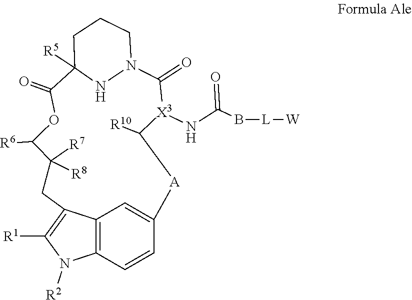

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula AI and subformula thereof, R⁶ is hydrogen.

In some embodiments of Formula AI and subformula thereof, R² is hydrogen, cyano, optionally substituted C₁-C₆ alkyl, optionally substituted 3 to 6-membered cycloalkyl, or optionally substituted 3 to 6-membered heterocycloalkyl. In some embodiments of Formula AI and subformula thereof, R² is optionally substituted C₁-C₆ alkyl, such as ethyl. In some embodiments of Formula AI and subformula thereof, R² is fluoro C₁-C₆ alkyl, such as —CH₂CH₂F, —CH₂CHF₂, or —CH₂CF₃.

In some embodiments of Formula AI and subformula thereof, R⁷ is optionally substituted C₁-C₃ alkyl. In some embodiments of Formula AI and subformula thereof, R⁷ is C₁-C₃ alkyl.

In some embodiments of Formula AI and subformula thereof, R⁸ is optionally substituted C₁-C₃ alkyl. In some embodiments of Formula AI and subformula thereof, R⁸ is C₁-C₃ alkyl, such as methyl.

In some embodiments, the RAS(ON) inhibitor has the structure of Formula Alf, or a pharmaceutically acceptable salt thereof:

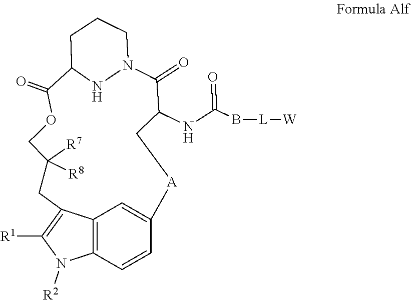

wherein A optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, R¹ is 5 to 10-membered heteroaryl.

In some embodiments, R¹ is optionally substituted 6-membered aryl or optionally substituted 6-membered heteroaryl.

In some embodiments of Formula AI and subformula thereof, R₁ is

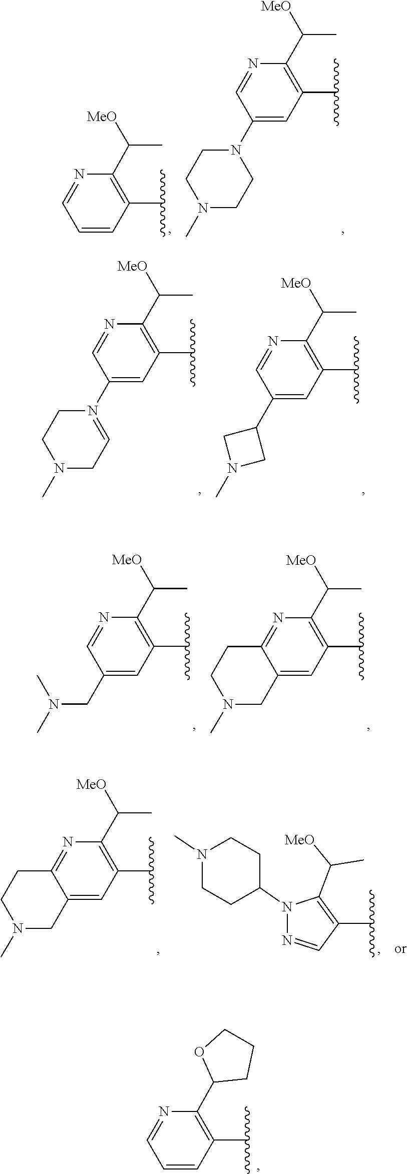

or a stereoisomer thereof. In some embodiments, R₁ is

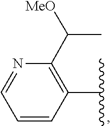 [SMILES: *c1cccnc1C(C)OC]

or a stereoisomer thereof. In some embodiments, R₁ is

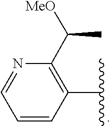 [SMILES: *c1cccnc1[C@H](C)OC]

In some embodiments, R₁ is

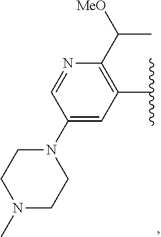 [SMILES: *c1cc(N2CCN(C)CC2)cnc1C(C)OC]

or a stereoisomer thereof. In some embodiments, R₁ is

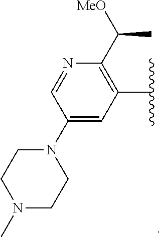 [SMILES: *c1cc(N2CCN(C)CC2)cnc1[C@H](C)OC]

In some embodiments, the RAS(ON) inhibitor has the structure of Formula AIg, or a pharmaceutically acceptable salt thereof:

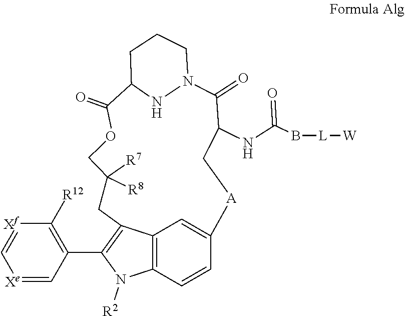

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is N, CH, or CR¹⁷;
X^{f} is N or CH;
R¹² is optionally substituted C₁-C₆ alkyl or optionally substituted C₁-C₆ heteroalkyl; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments of Formula AI and subformula thereof, X^{e} is N and X^{f} is CH. In some embodiments, X^{e} is CH and X^{f} is N. In some embodiments, X^{e} is CR¹⁷ and X¹ is N.

In some embodiments of Formula AI and subformula thereof, R¹² is optionally substituted C₁-C₆ heteroalkyl. In some embodiments, R¹² is

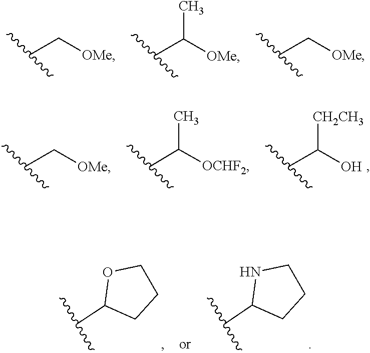

In some embodiments, the RAS(ON) inhibitor has the structure of Formula AIh, or a pharmaceutically acceptable salt thereof:

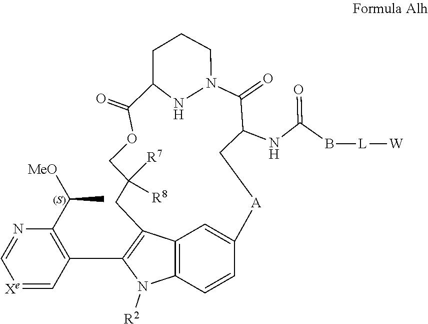

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} is CH, or CR¹⁷; and
R¹⁷ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS(ON) inhibitor has the structure of Formula Ali, or a pharmaceutically acceptable salt thereof:

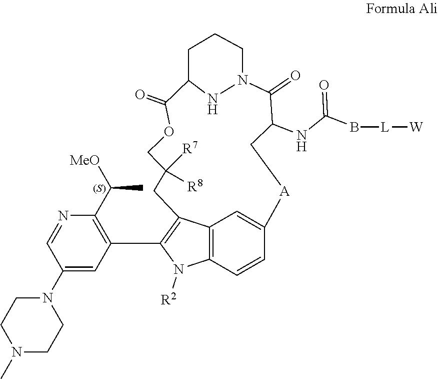

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, A is optionally substituted 6-membered arylene. In some embodiments, A has the structure:

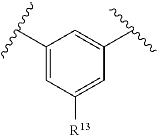

wherein R¹³ is hydrogen, hydroxy, amino, cyano, optionally substituted C₁-C₆ alkyl, or optionally substituted C₁-C₆ heteroalkyl. In some embodiments, R¹³ is hydrogen. In some embodiments, R¹³ is hydroxy. In some embodiments, A is an optionally substituted 5 to 10-membered heteroarylene. In some embodiments, A is:

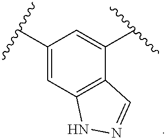 [SMILES: *c1cc(*)c2cn[nH]c2c1]

In some embodiments, A is optionally substituted 5 to 6-membered heteroaylene. In some embodiments, A is:

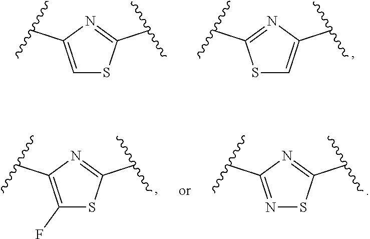 [SMILES: *c1csc(*)n1 ; *c1csc(*)n1 ; *c1nc(*)c(F)s1 ; *c1nsc(*)n1]

In some embodiments, A is

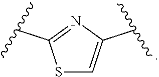 [SMILES: *c1csc(*)n1]

In some embodiments of Formula AI and subformula thereof, B is —CHR⁹—. In some embodiments, R⁹ is optionally substituted C₁-C₆ alkyl or optionally substituted 3 to 6-membered cycloalkyl. In some embodiments, R⁹ is:

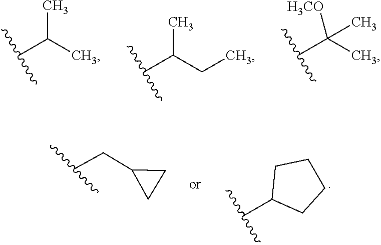

In some embodiments, R⁹ is:

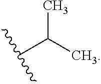 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula AI and subformula thereof, B is optionally substituted 6-membered arylene.

In some embodiments, B is 6-membered arylene. In some embodiments, B is:

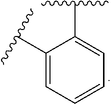 [SMILES: *c1ccccc1*]

In some embodiments B is absent.

In some embodiments of Formula AI and subformula thereof, R⁷ is methyl.

In some embodiments of Formula AI and subformula thereof, R⁸ is methyl.

In some embodiments of Formula AI and subformula thereof, R¹⁶ is hydrogen.

In some embodiments of Formula AI and subformula thereof, the linker is the structure of Formula AII:

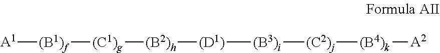

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₁-C₃ cycloalkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D1 is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments, the linker is acyclic. In some embodiments, the linker has the structure of Formula AIIa:

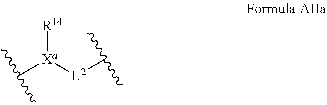

wherein X^{a} is absent or N;
R¹⁴ is absent, hydrogen or optionally substituted C₁-C₆ alkyl or optionally substituted C₁-C₃ cycloalkyl; and
L² is absent, —C(O)—, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

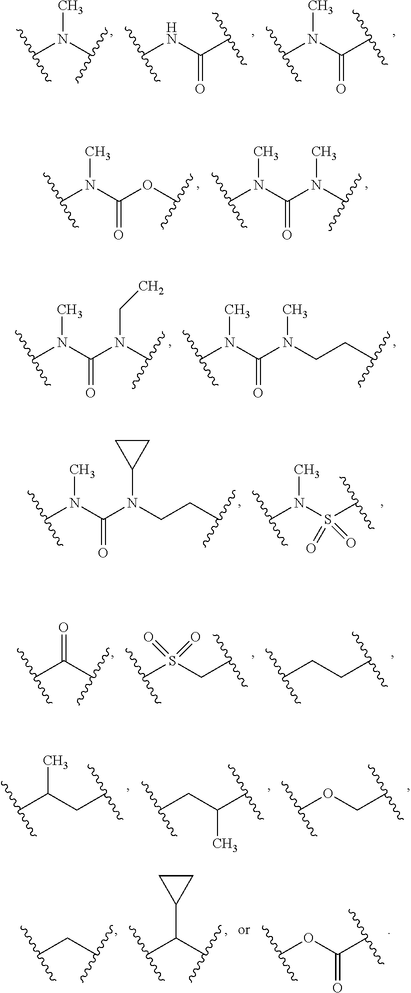

In some embodiments, L is

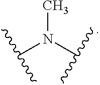 [SMILES: *N(*)C]

In some embodiments, L is

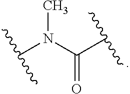 [SMILES: *C(=O)N(*)C]

In some embodiments, linker is or comprises a cyclic group. In some embodiments of Formula AI and subformula thereof, the linker has the structure of Formula AIIb:

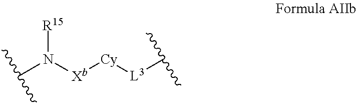

wherein o is 0 or 1;
X^{b} is C(O) or SO₂;
R¹⁵ is hydrogen or optionally substituted C₁-C₆ alkyl;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —C(O)—, —SO₂—, optionally substituted C₁-C₄ alkylene or optionally substituted C₁-C₄ heteroalkylene. In some embodiments, the linker has the structure:

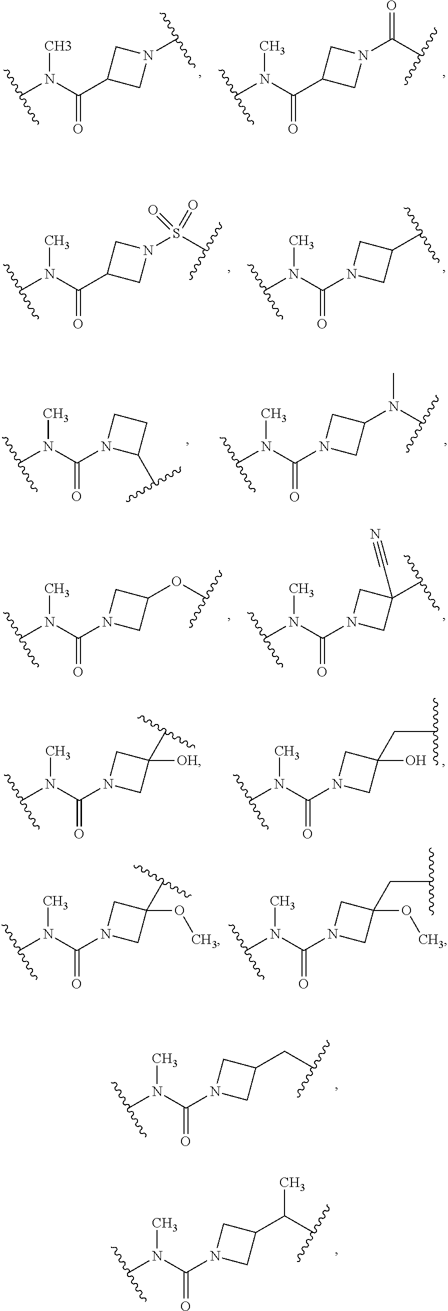

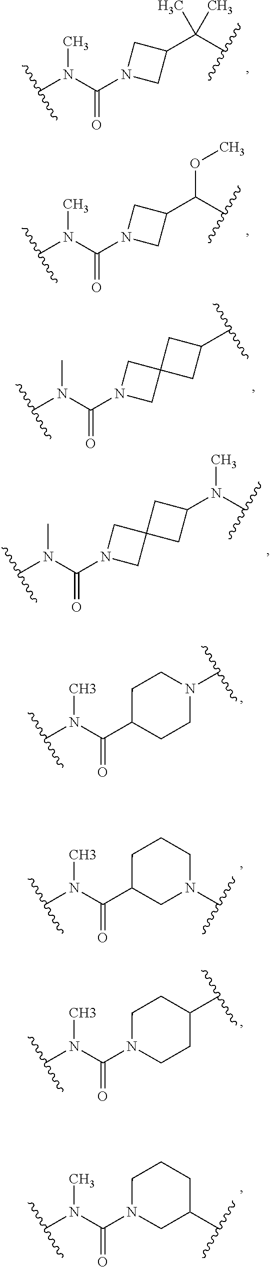

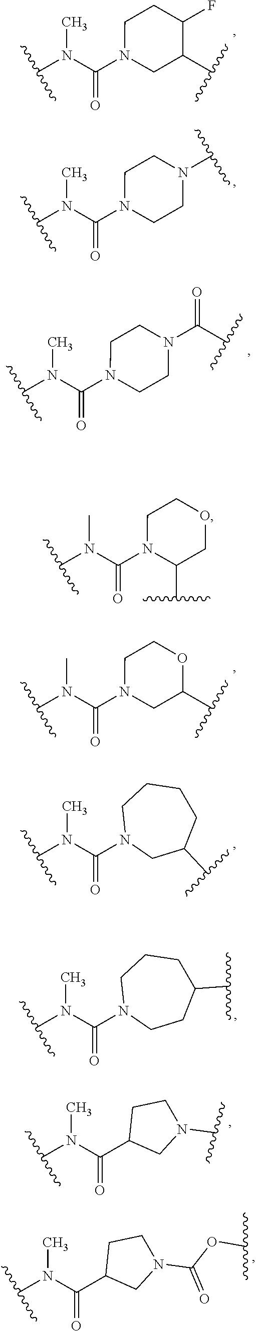

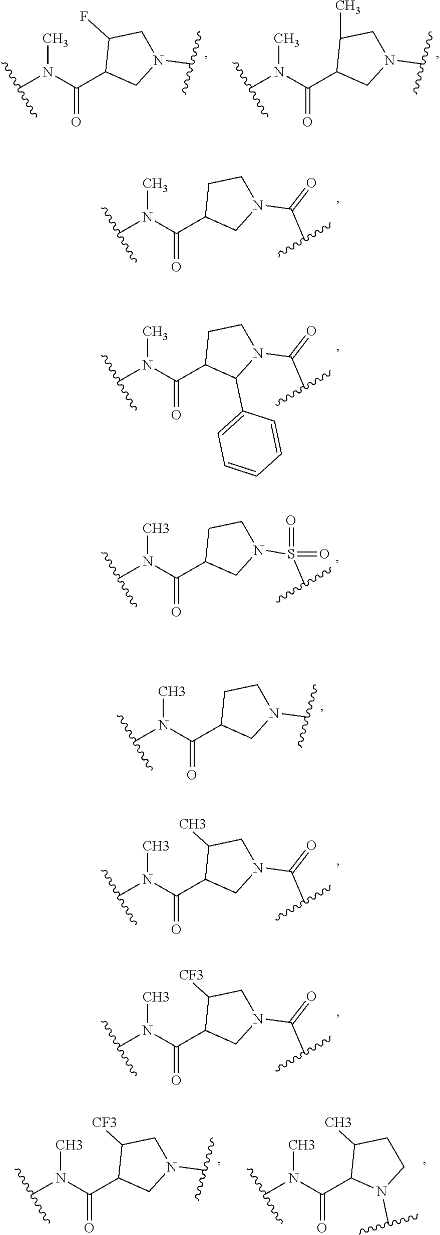

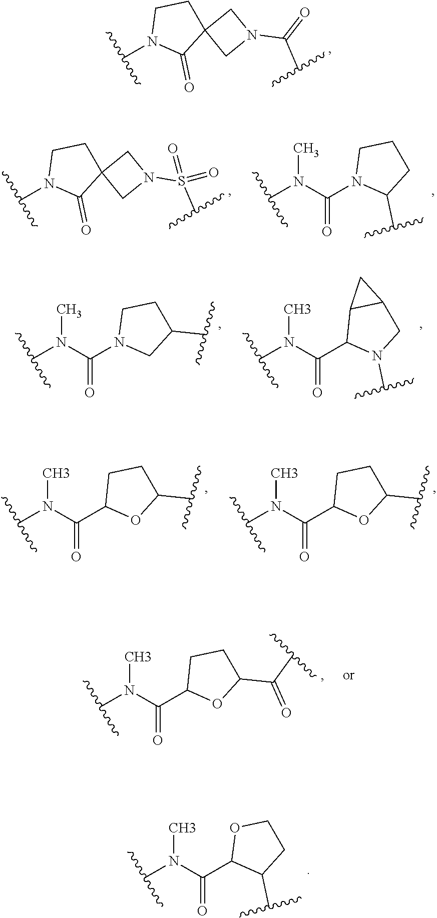

In some embodiments of Formula AI and subformula thereof, W is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 8-membered heterocycloalkyl, optionally substituted 3 to 8-membered cycloalkyl, or 3 to 8-membered heteroaryl.

In some embodiments of Formula AI and subformula thereof, W is hydrogen. In some embodiments of Formula AI and subformula thereof, W is optionally substituted amino. In some embodiments of Formula AI and subformula thereof, W is —NHCH₃ or —N(CH₃)₂. In some embodiments of Formula AI and subformula thereof, W is optionally substituted C₁-C₄ alkoxy. In some embodiments, W is methoxy or iso-propoxy. In some embodiments of Formula AI and subformula thereof, W is optionally substituted C₁-C₄ alkyl. In some embodiments, W is methyl, ethyl, iso-propyl, tert-butyl, or benzyl. In some embodiments of Formula AI and subformula thereof, W is optionally substituted amido. In some embodiments, W is

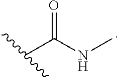 [SMILES: *C(=O)NC]

In some embodiments, W is

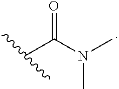 [SMILES: *C(=O)N(C)C]

In some embodiments of Formula AI and subformula thereof, W is optionally substituted C₁-C₄ hydroxyalkyl. In some embodiments, W is

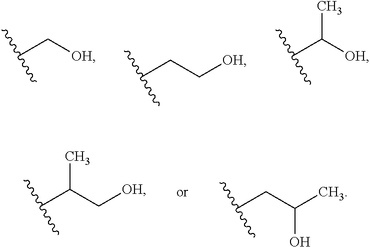

In some embodiments of Formula AI and subformula thereof, W is optionally substituted C₁-C₄ aminoalkyl. In some embodiments, W is

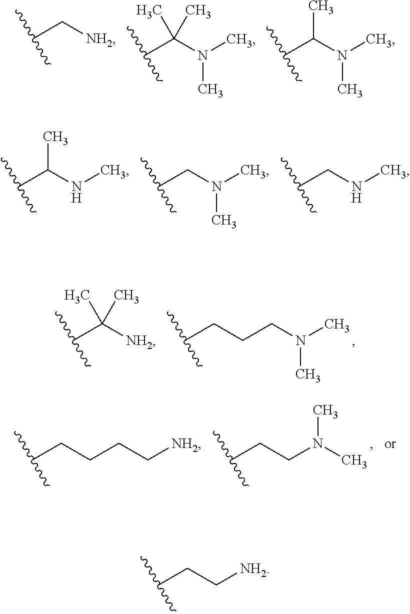

In some embodiments of Formula AI and subformula thereof, W is optionally substituted C₁-C₄ haloalkyl. In some embodiments, W is

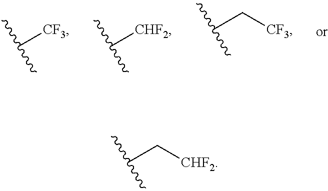

In some embodiments of Formula AI and subformula thereof, W is optionally substituted C₁-C₄ guanidinoalkyl. In some embodiments, W is

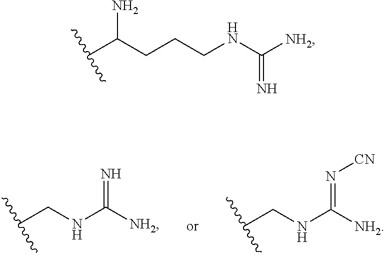 [SMILES: *C(N)CCCNC(=N)N ; *CNC(=N)N ; *CN/C(N)=N/C#N]

In some embodiments of Formula AI and subformula thereof, W is C₀-C₄ alkyl optionally substituted 3 to 11-membered heterocycloalkyl. In some embodiments, W is

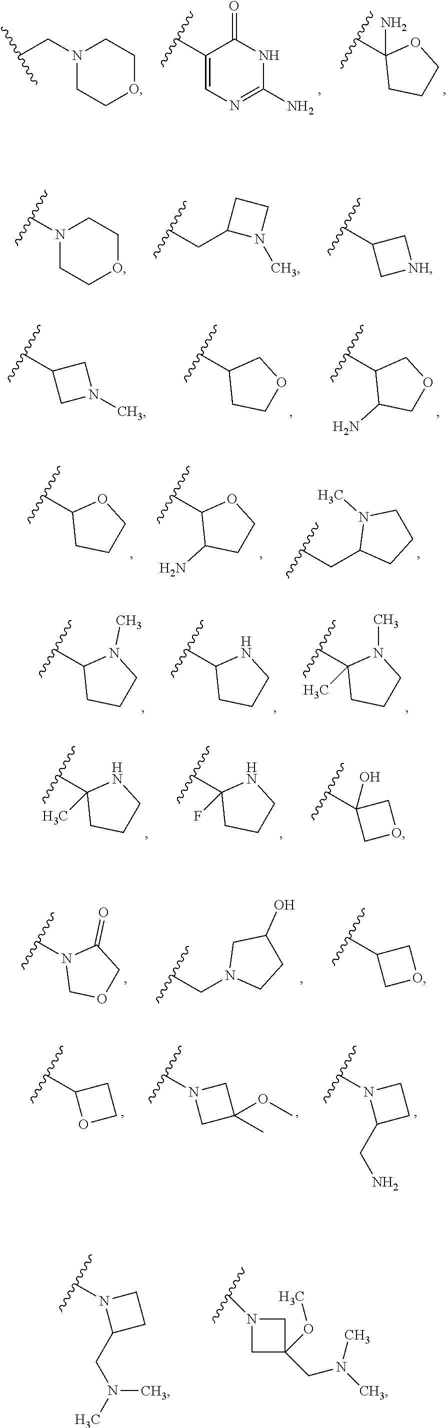

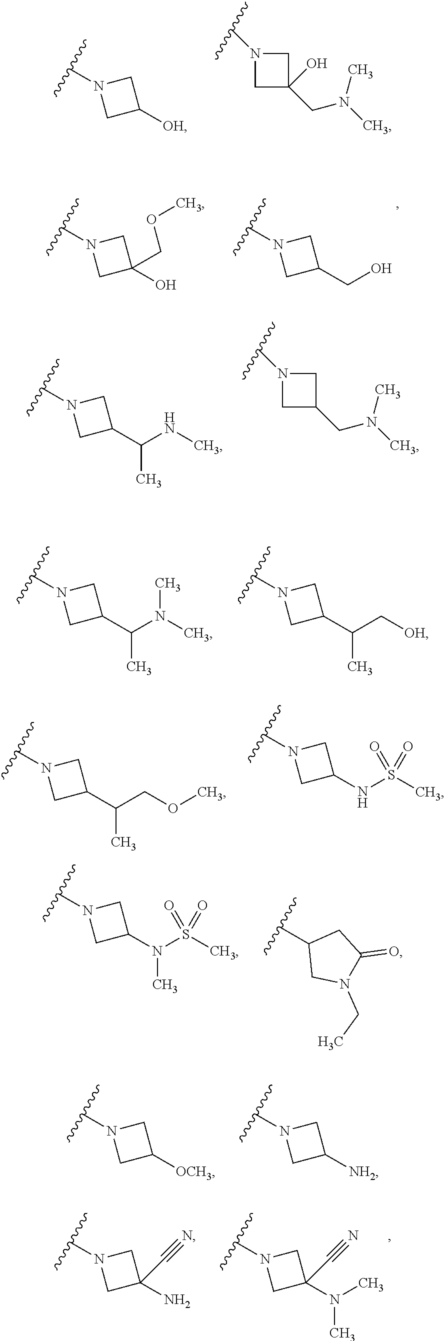

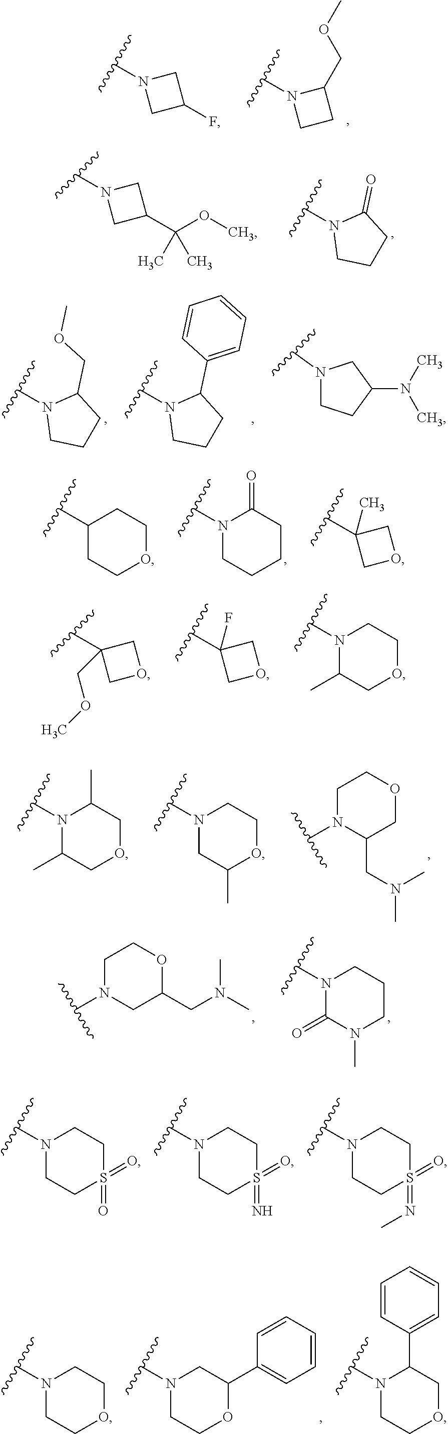

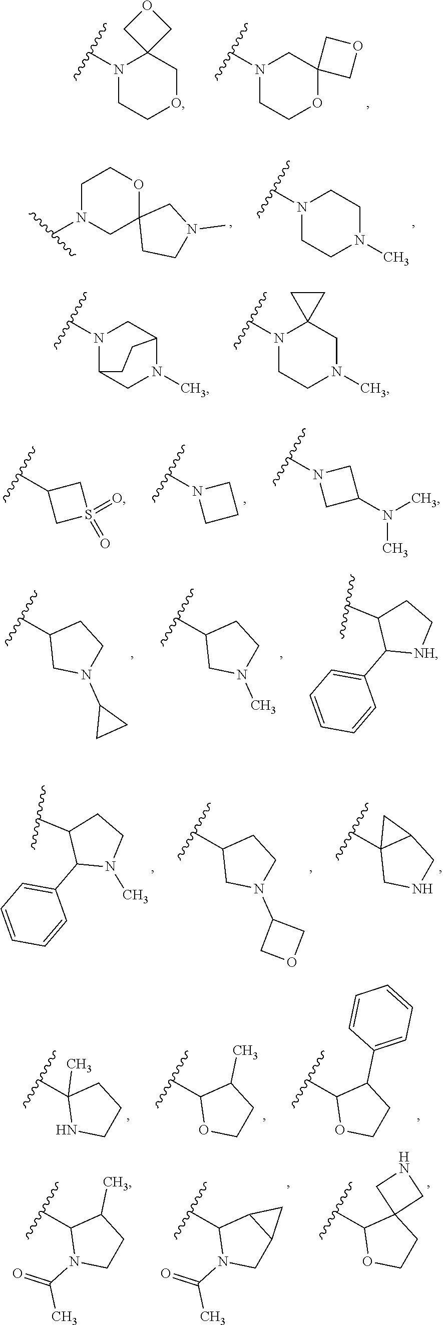

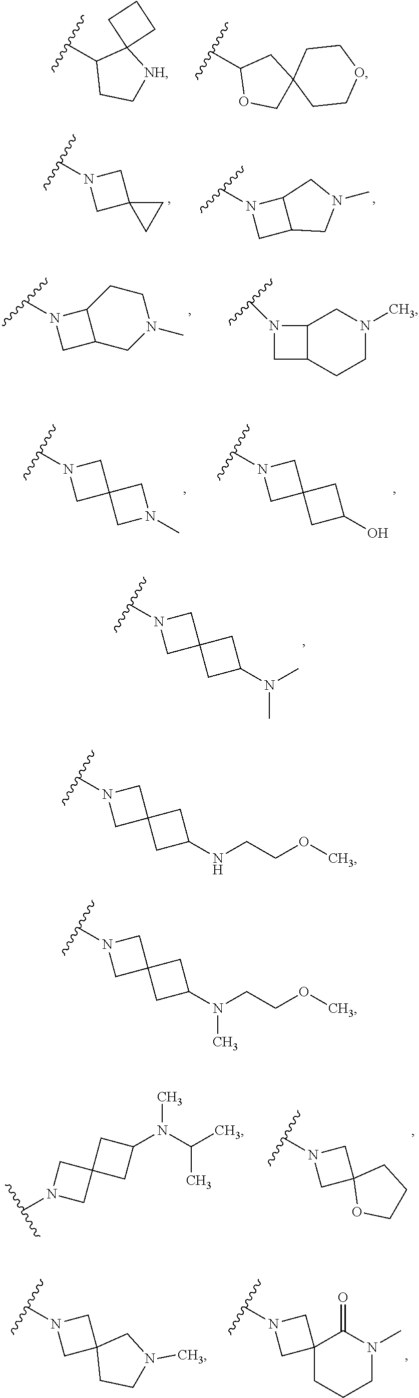

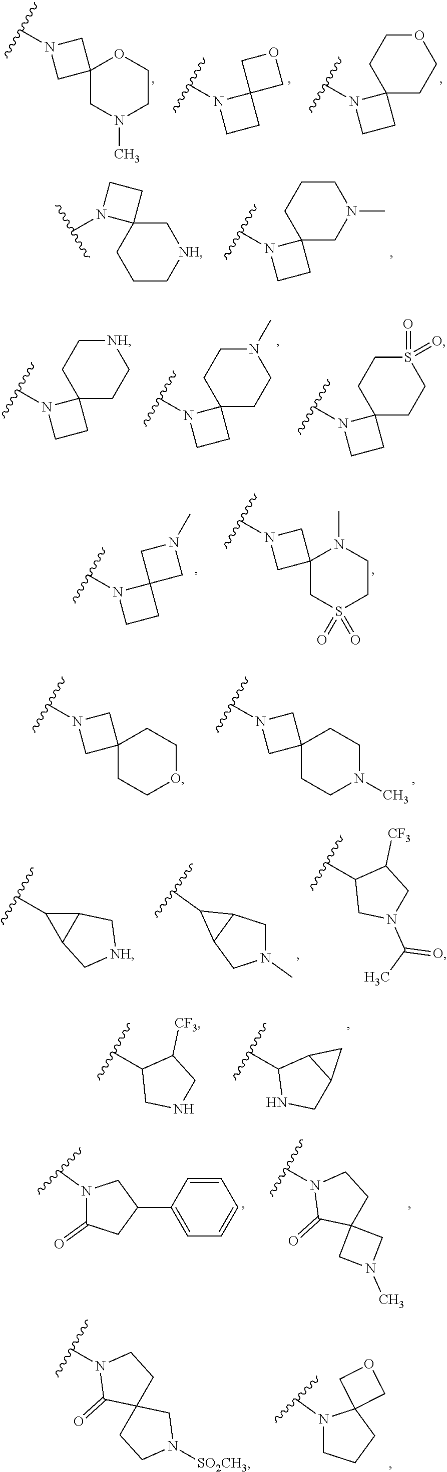

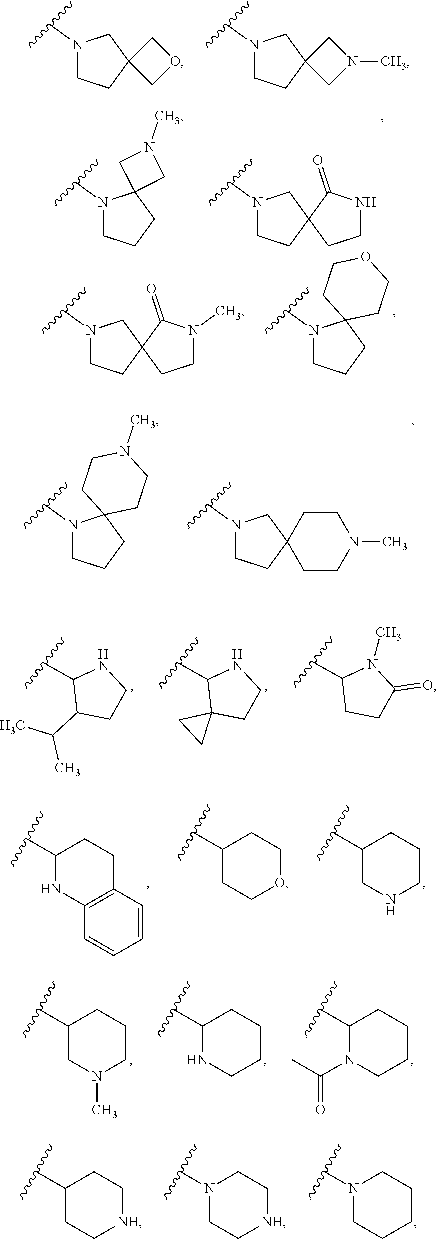

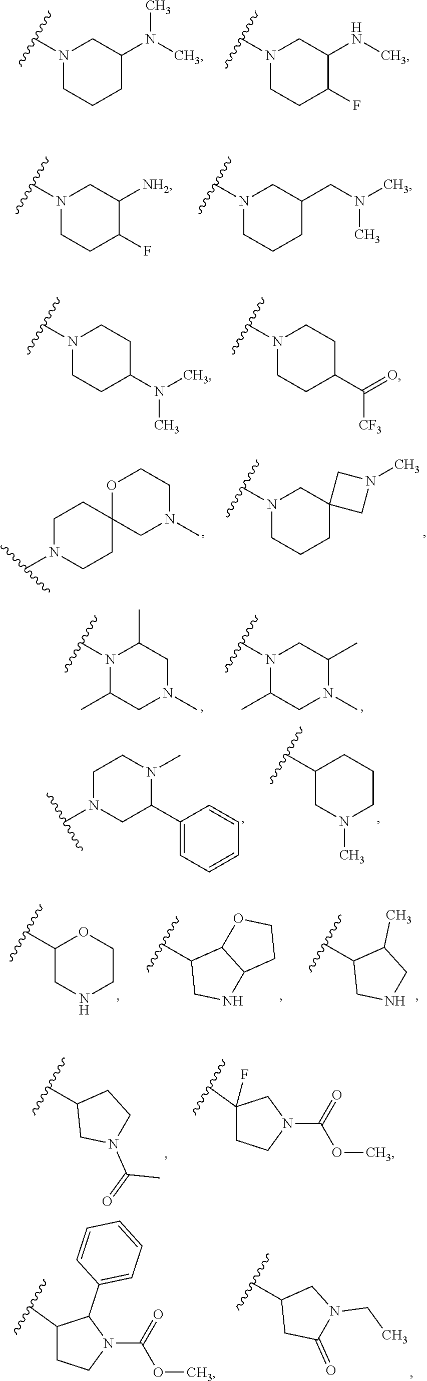

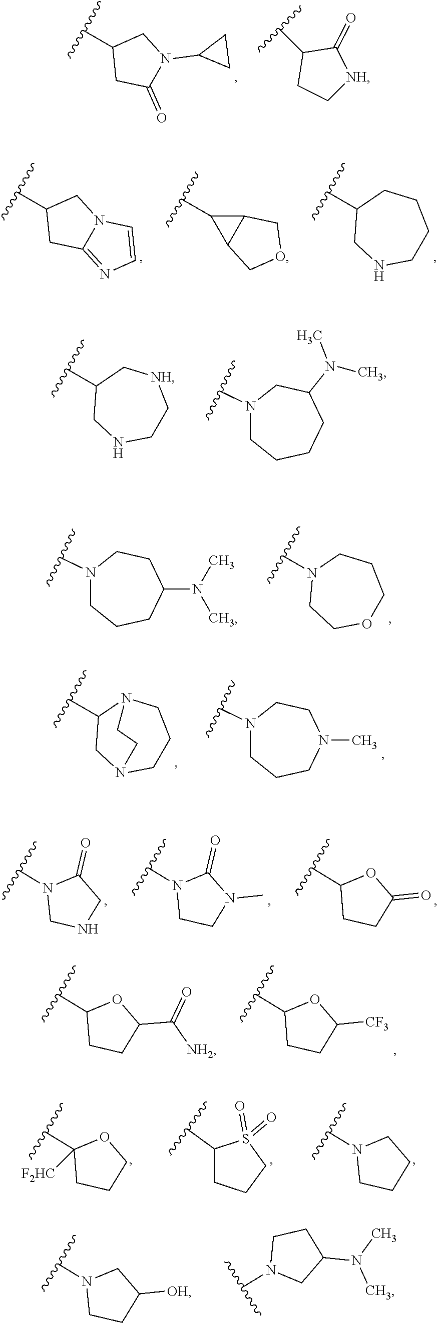

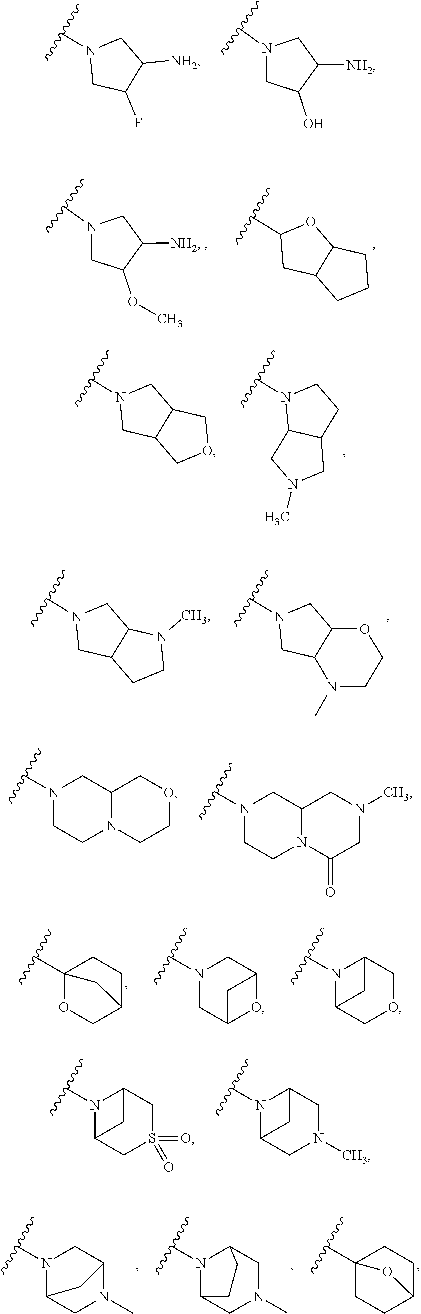

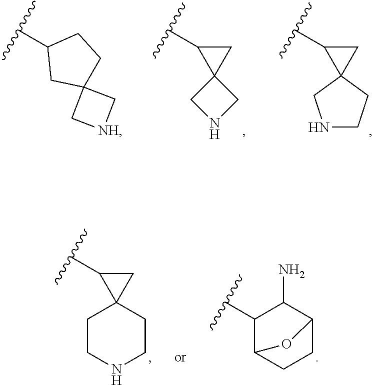 [SMILES: *C1CCC2(CNC2)C1 ; *C1CC12CNC2 ; *C1CC12CCNC2 ; *C1CC12CCNCC2 ; *C1C2CCC(O2)C1N]

In some embodiments of Formula AI and subformula thereof, W is optionally substituted 3 to 8-membered cycloalkyl. In some embodiments, W is

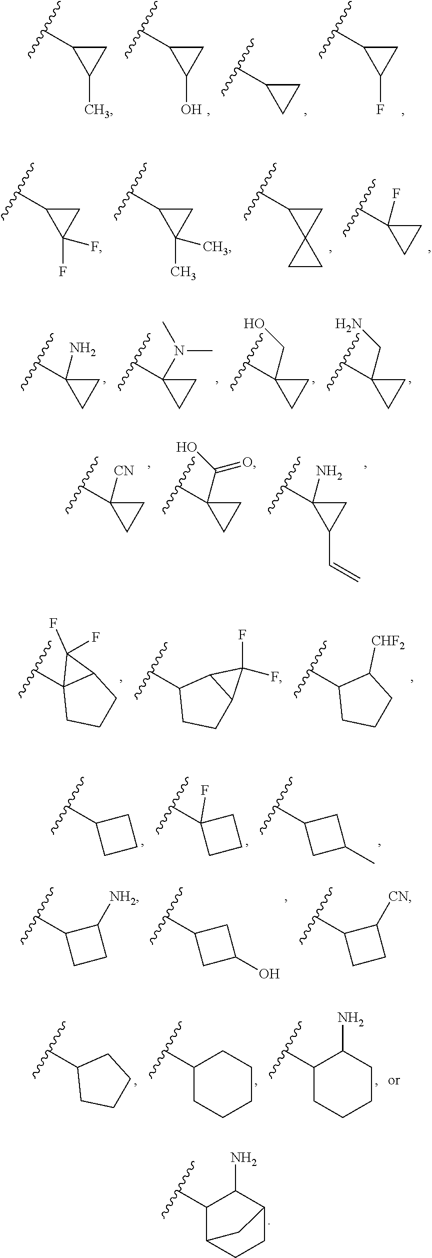

In some embodiments of Formula AI and subformula thereof, W is optionally substituted 3 to 8-membered heteroaryl. In some embodiments, W is

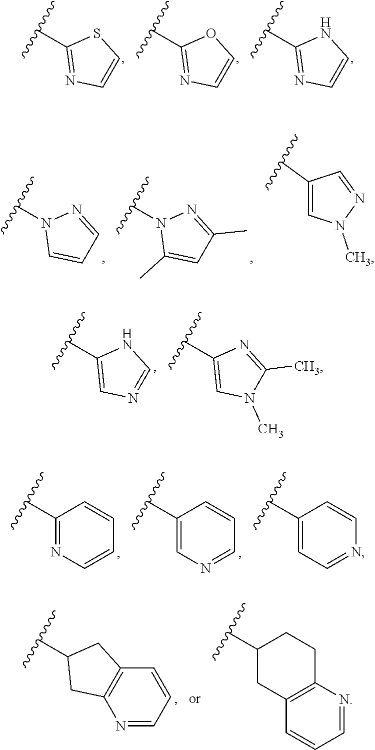

In some embodiments of Formula AI and subformula thereof, W is optionally substituted 6- to 10-membered aryl (e.g., phenyl, 4-hydroxy-phenyl, or 2,4-methoxy-phenyl).

In some embodiments, the RAS(ON) inhibitor is selected from Table A1, or a pharmaceutically acceptable salt or stereoisomer thereof. In some embodiments, the RAS(ON) inhibitor is selected from Table A1, or a pharmaceutically acceptable salt or atropisomer thereof.

TABLE A1
Certain Compounds of the Present Invention

| Ex # | Structure |
|---|---|
| AA1 | 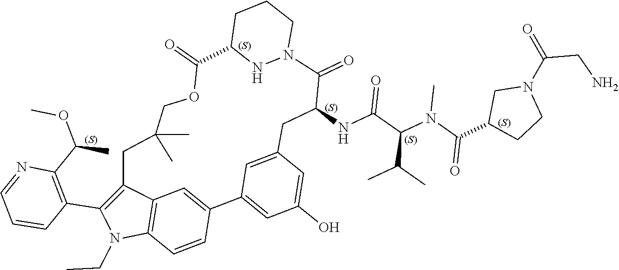 |
| AA2 | 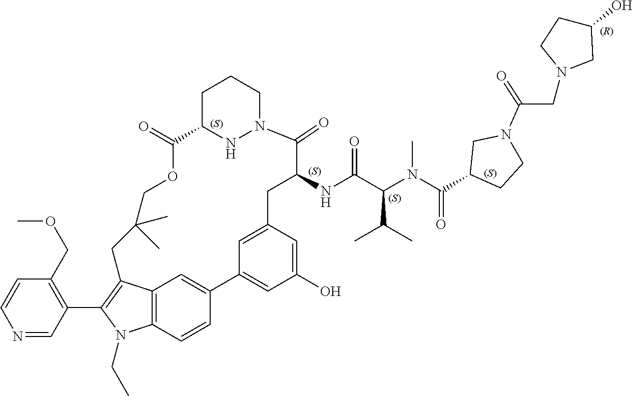 |
| AA3 | 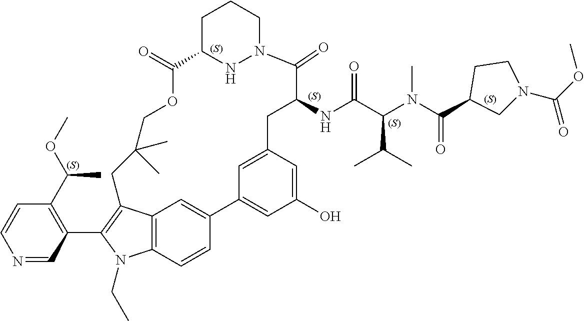 |
| AA4 | 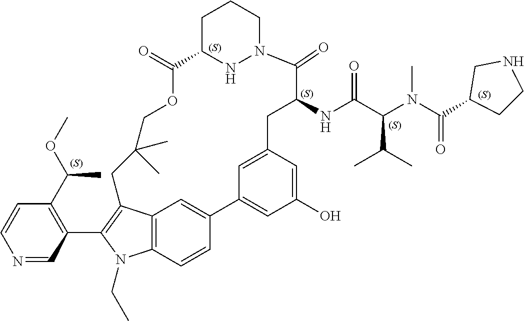 |
| AA5 | 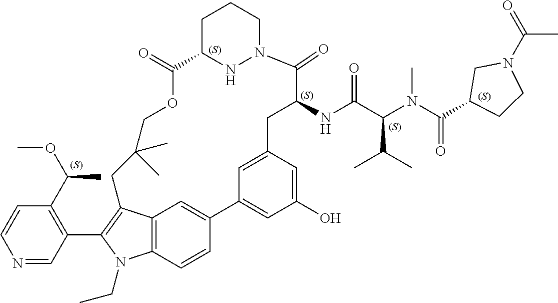 |
| AA6 | 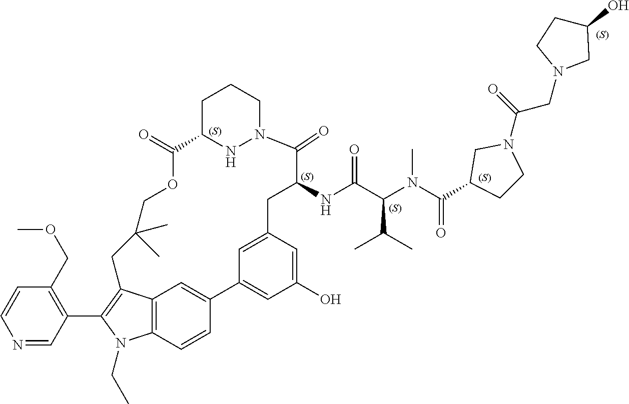 |
| AA7 | 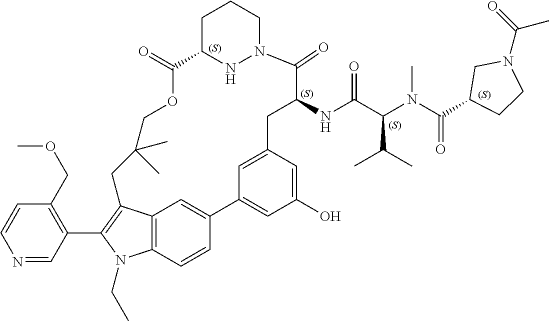 |
| AA8 | 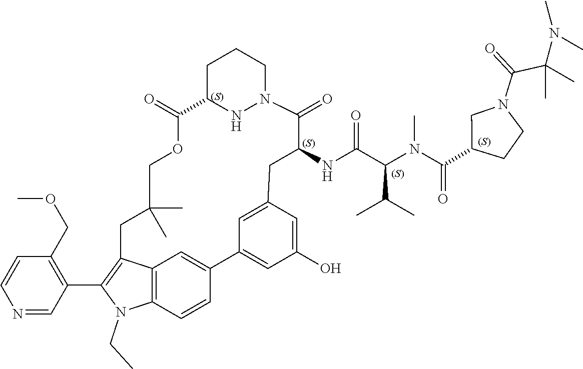 |
| AA9 | 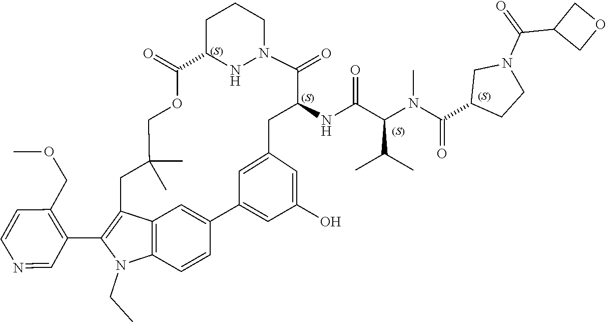 |
| AA10 | 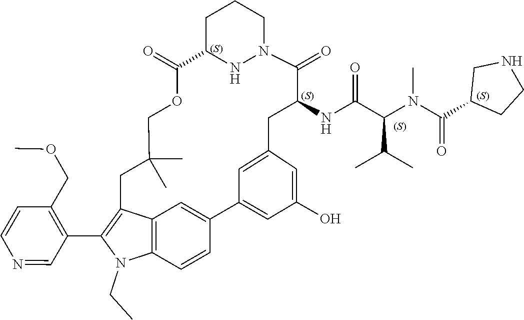 |
| AA11 | 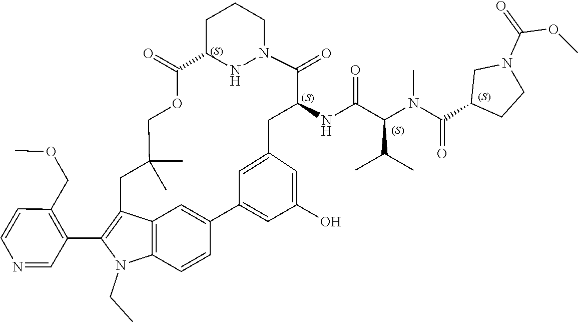 |
| AA12 | 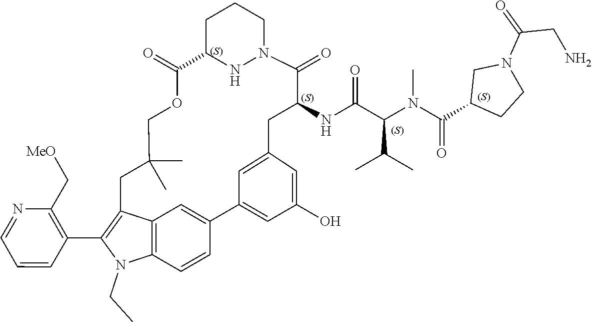 |
| AA13 | 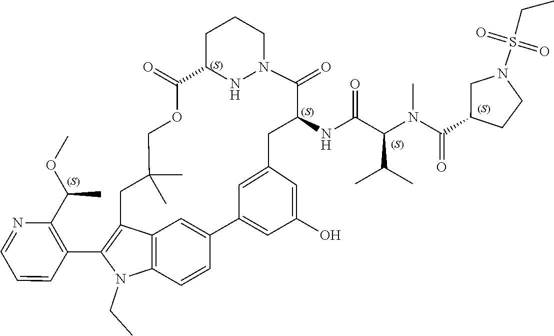 |
| AA14 | 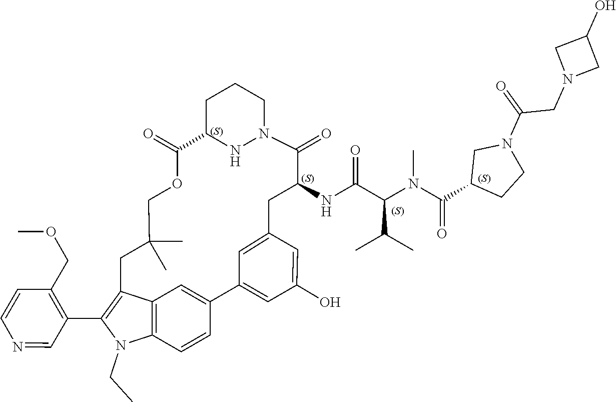 |
| AA15 | 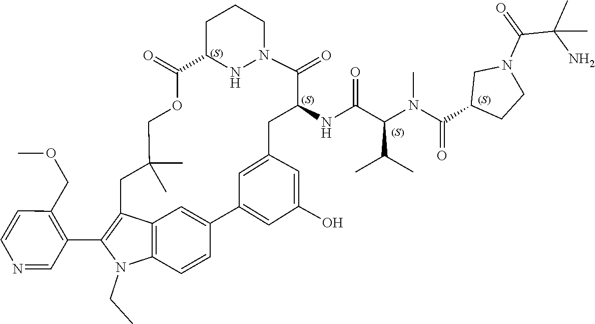 |
| AA16 | 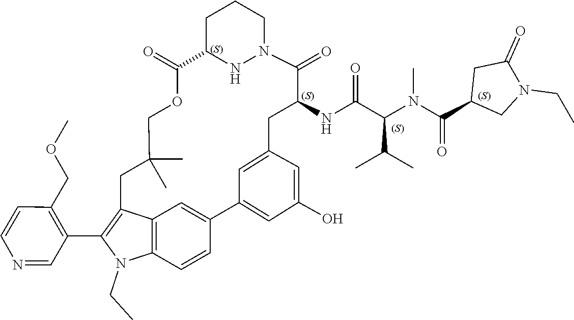 |
| AA17 | 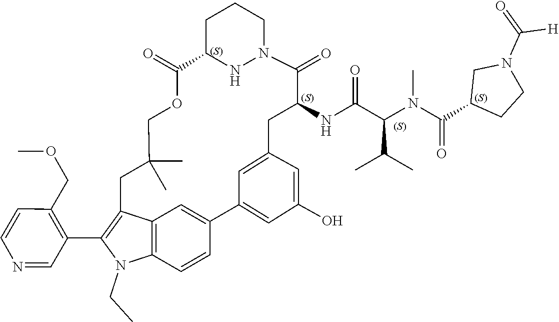 |
| AA18 | 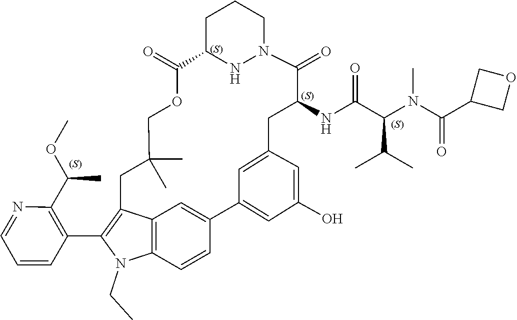 |
| AA19 | 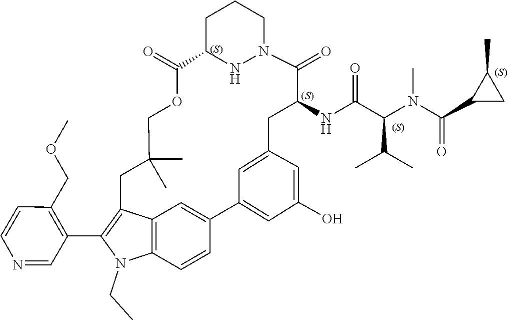 |
| AA20 | 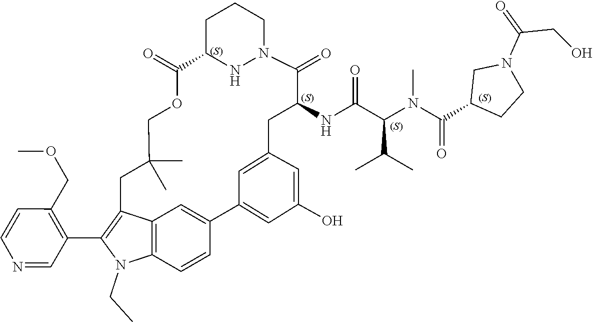 |
| AA21 | 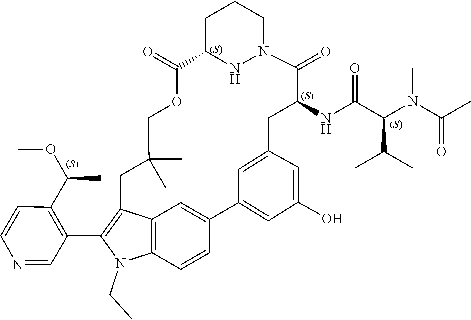 |
| AA22 | 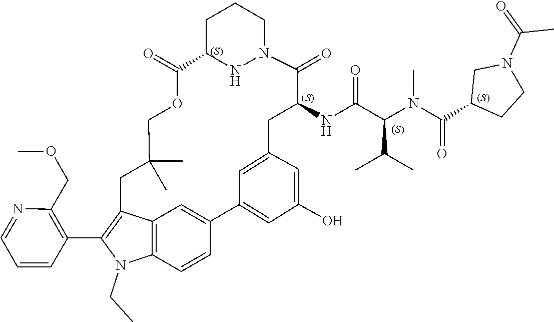 |
| AA23 | 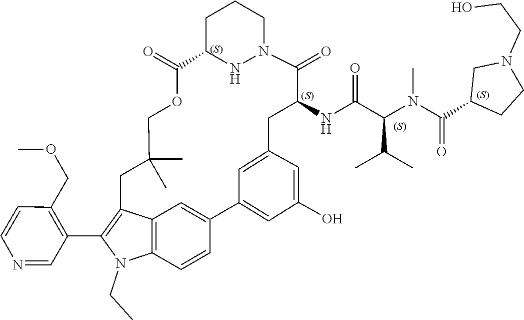 |
| AA24 | 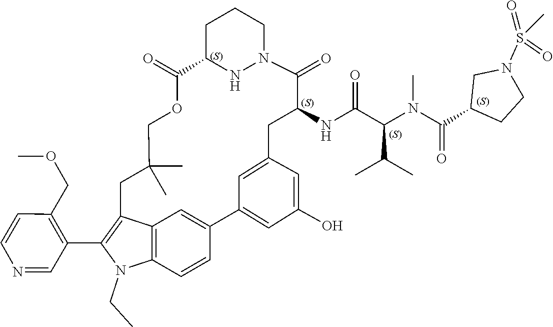 |
| AA25 | 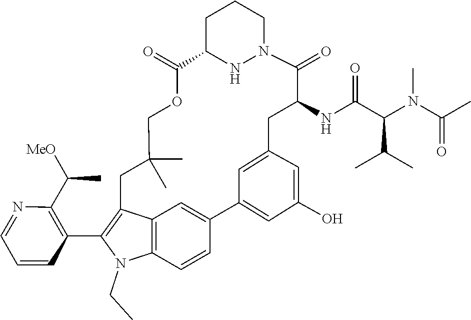 |
| AA26 | 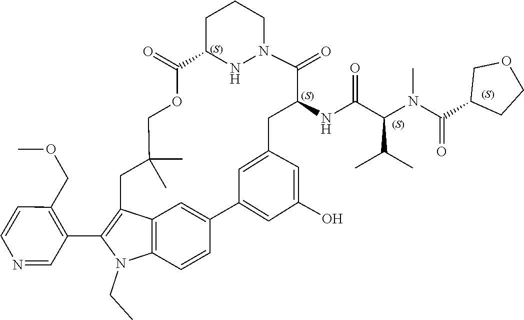 |
| AA27 | 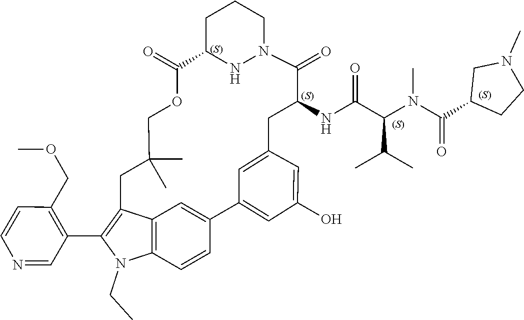 |
| AA28 | 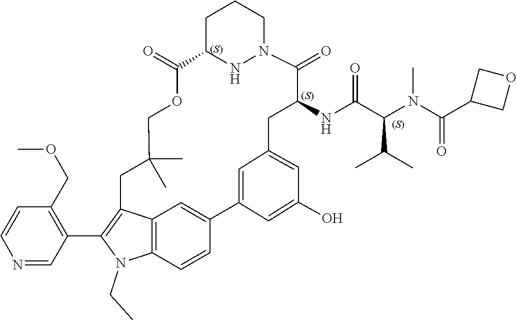 |
| AA29 | 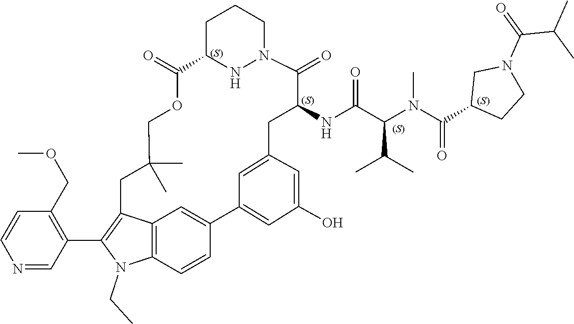 |
| AA30 | 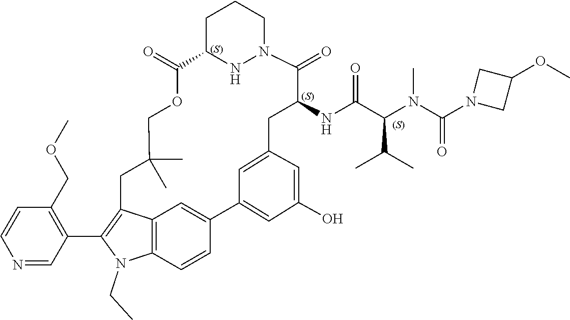 |
| AA31 | 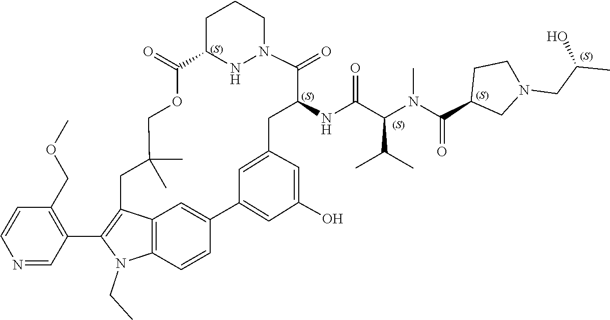 |
| AA32 | 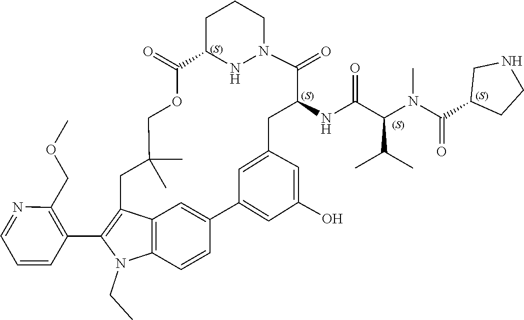 |
| AA33 | 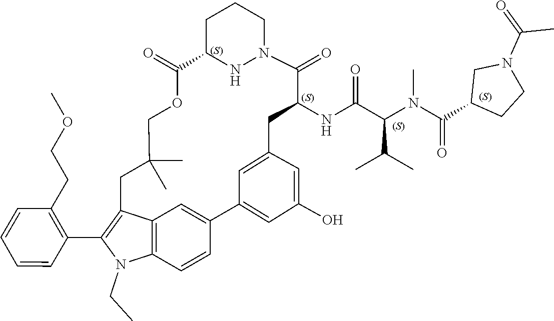 |
| AA34 | 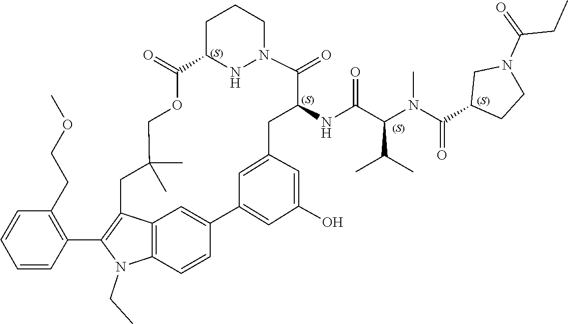 |
| AA35 | 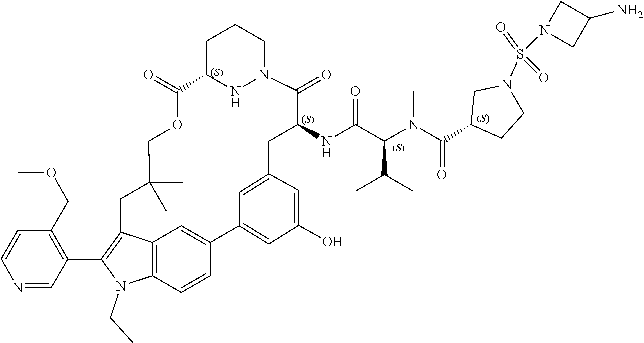 |
| AA36 | 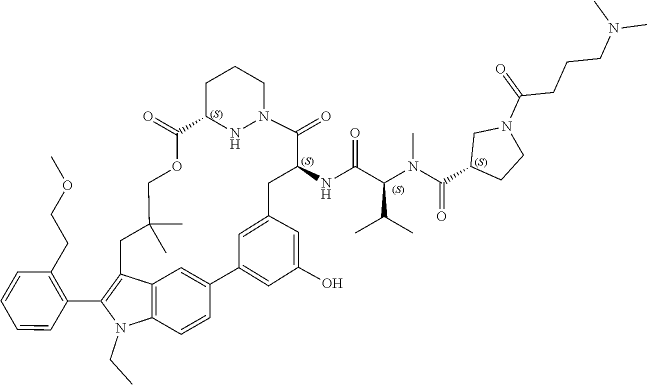 |
| AA37 | 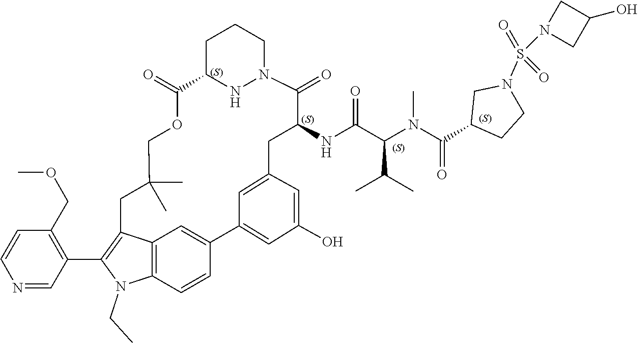 |
| AA38 | 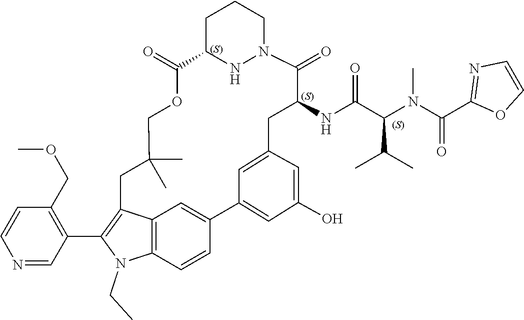 |
| AA39 | 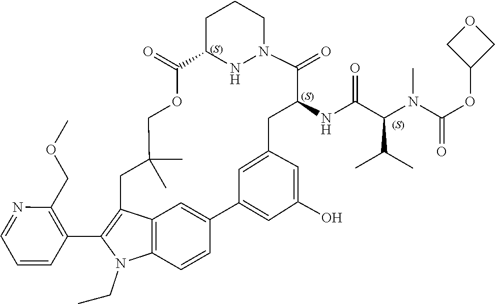 |
| AA40 | 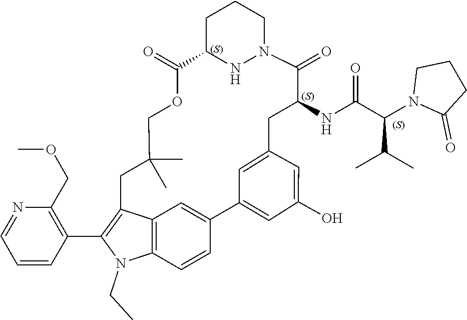 |
| AA41 | 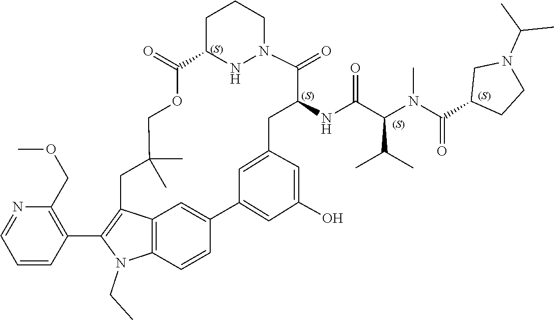 |
| AA42 | 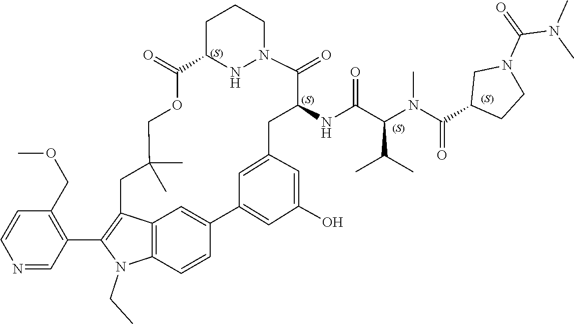 |
| AA43 | 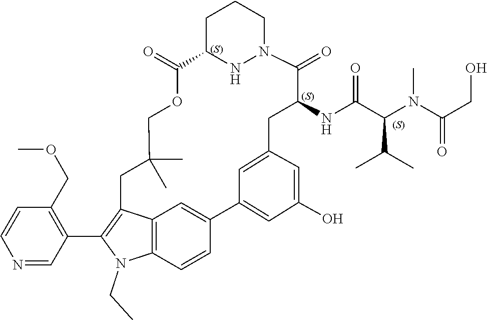 |
| AA44 | 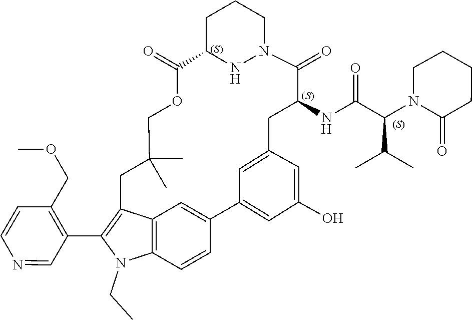 |
| AA45 | 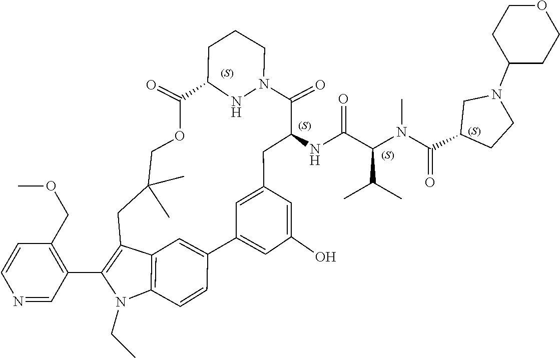 |
| AA46 | 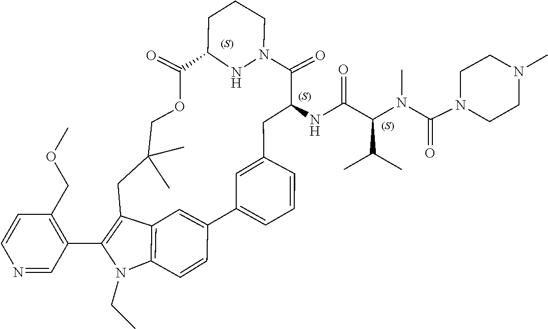 |
| AA47 | 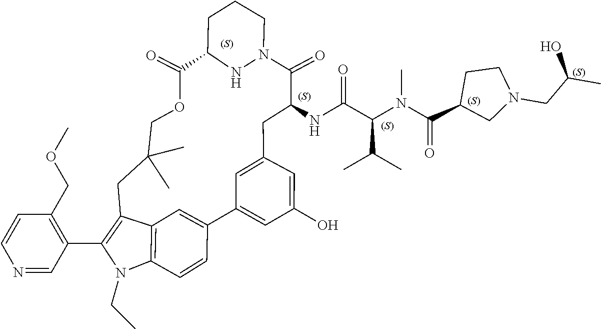 |
| AA48 | 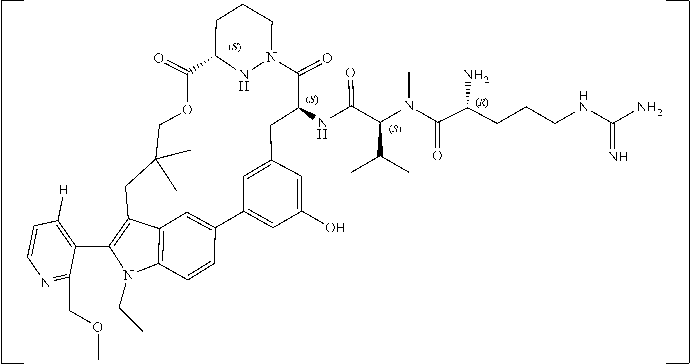 |
| AA49 | 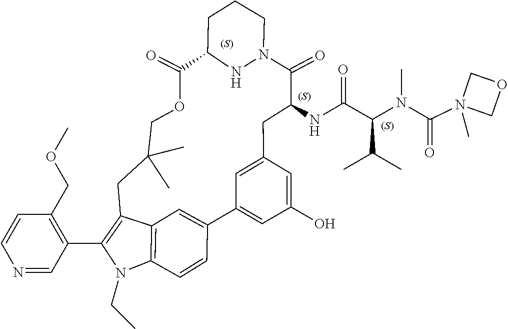 |
| AA50 | 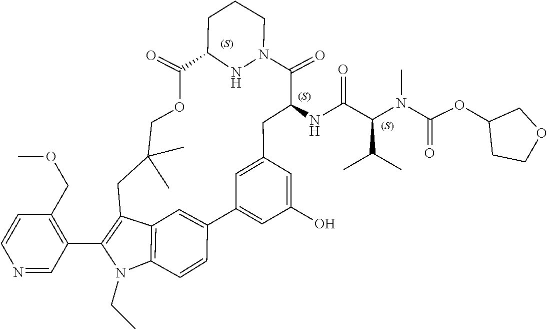 |
| AA51 | 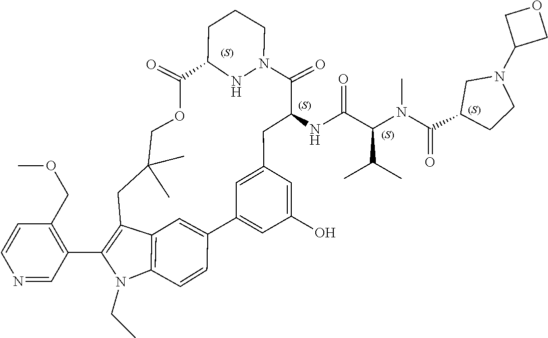 |
| AA52 | 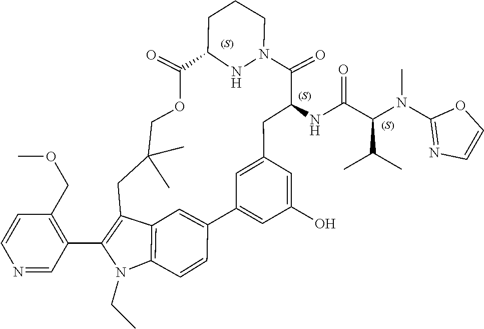 |
| AA53 | 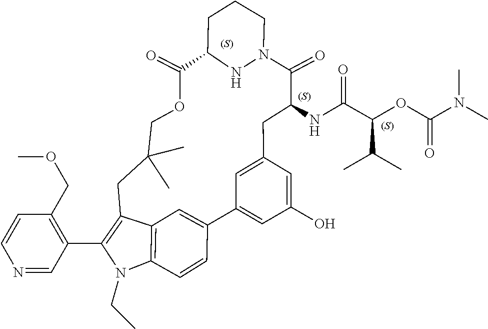 |
| AA54 | 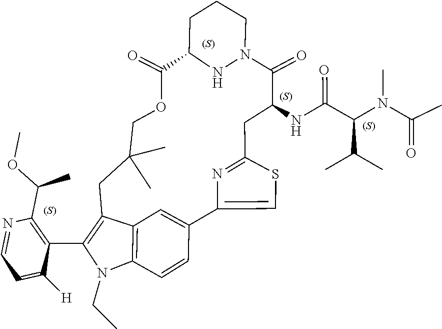 |
| AA55 | 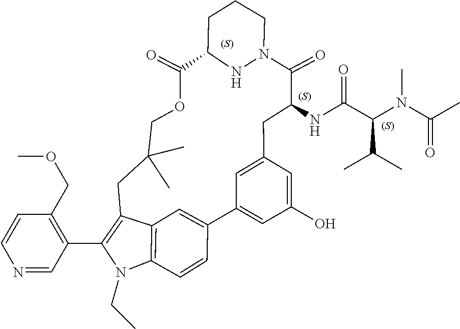 |
| AA56 | 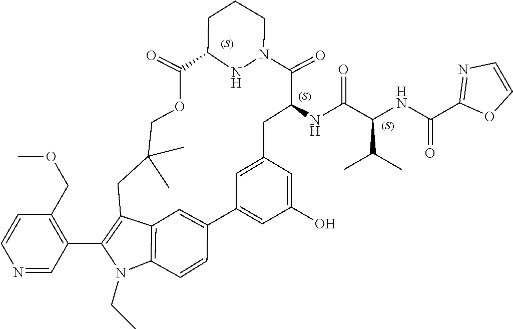 |
| AA57 | 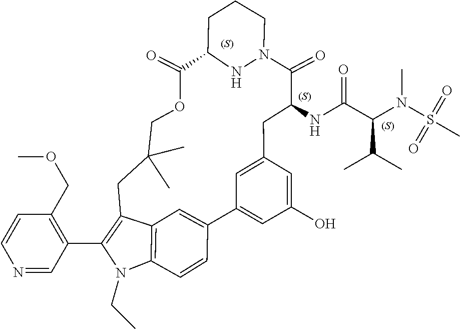 |
| AA58 | 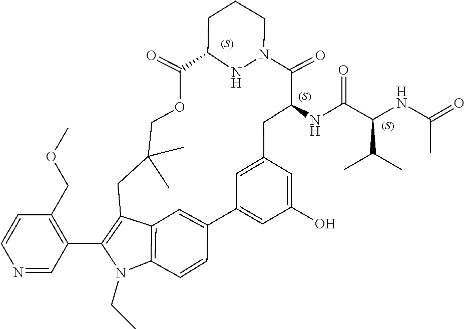 |
| AA59 | 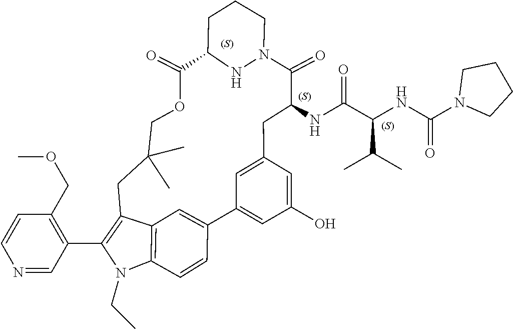 |
| AA60 | 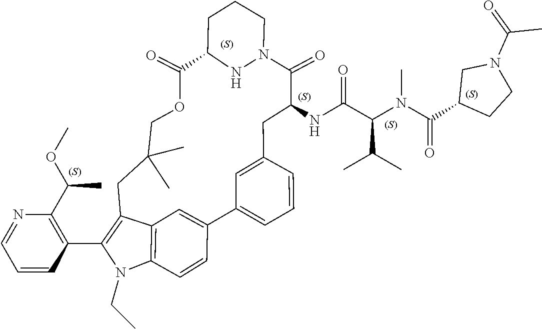 |
| AA61 | 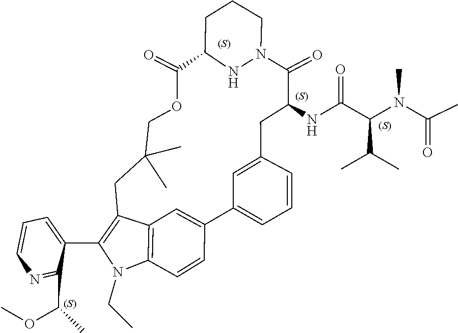 |
| AA62 | 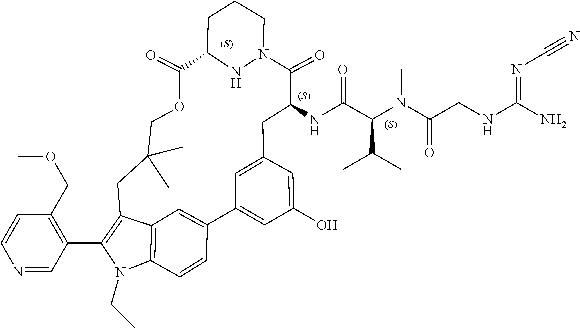 |
| AA63 | 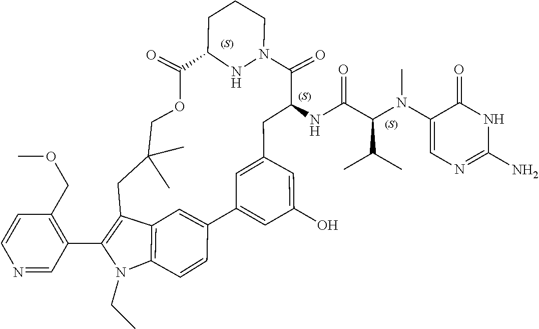 |
| AA64 | 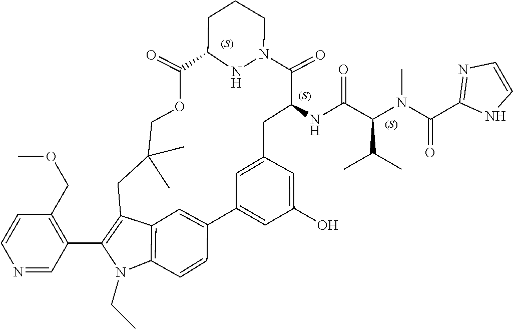 |
| AA65 | 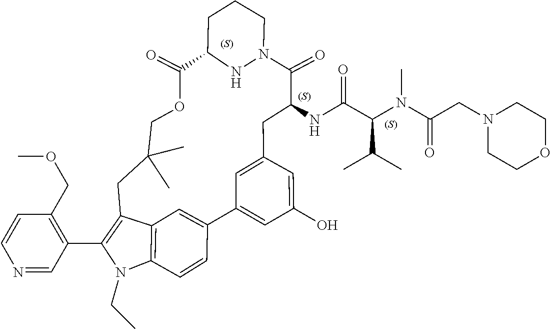 |
| AA66 | 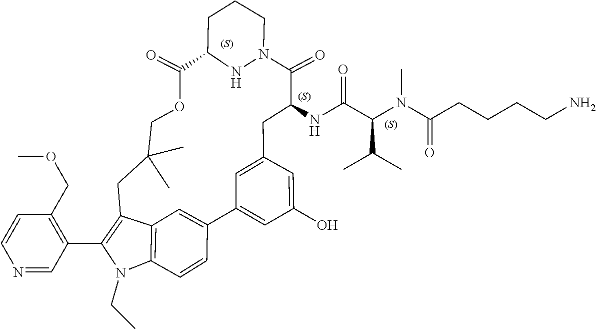 |
| AA67 | 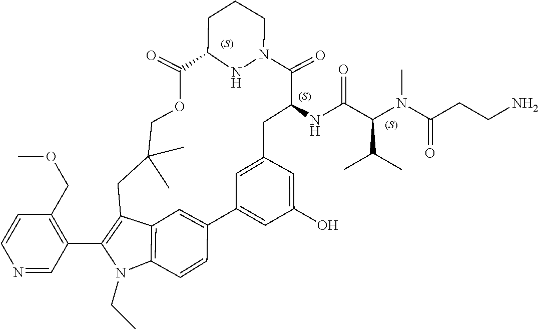 |
| AA68 | 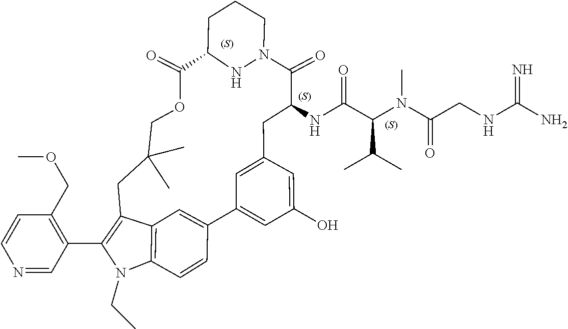 |
| AA69 | 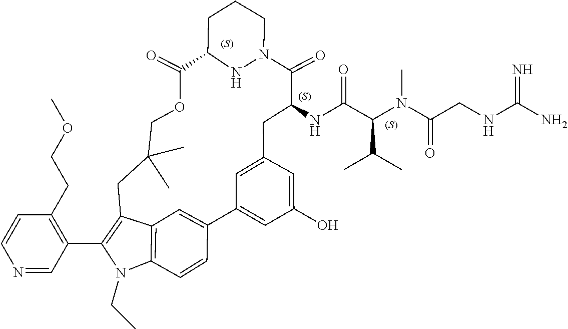 |
| AA70 | 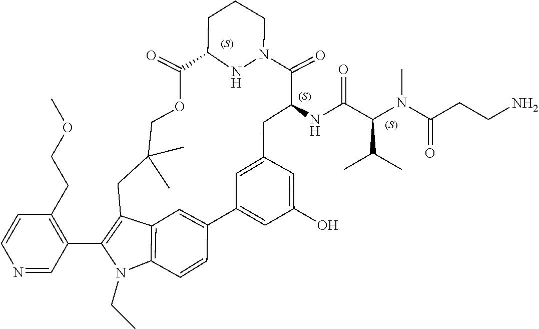 |
| AA71 | 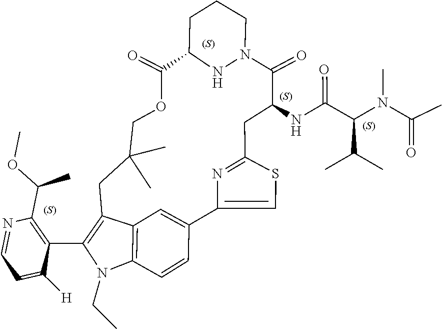 |
| AA72 | 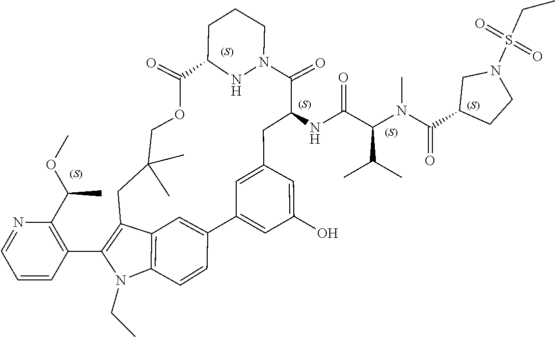 |
| AA73 | 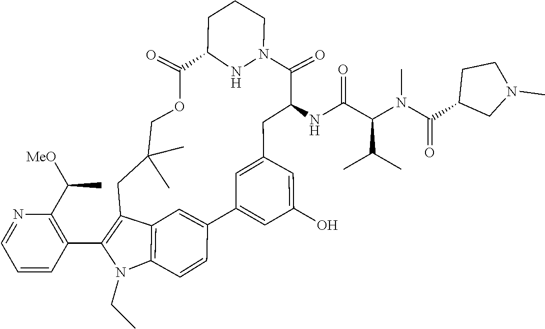 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CCN(C)C1)C(=O)N1CCC[C@H](N1)C(=O)OCC(C)(C)C2] |
| AA74 | 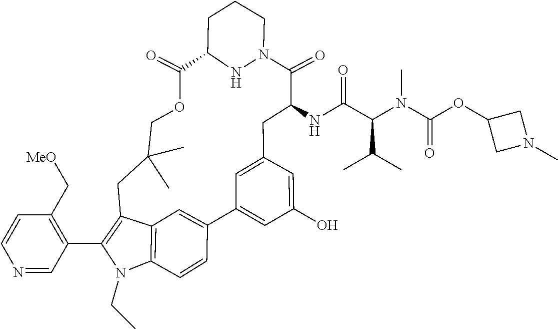 [SMILES: CCn1c(-c2cnccc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| AA75 | 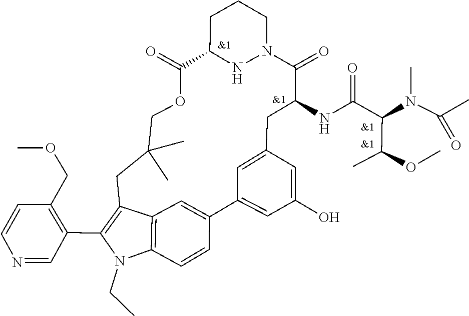 [SMILES: CCn1c(-c2cnccc2COC)c2c3cc(ccc31)-c1cc(O)cc(c1)C[C@H](NC(=O)[C@H]([C@H](C)OC)N(C)C(C)=O)C(=O)N1CCC[C@H](N1)C(=O)OCC(C)(C)C2] |
| AA76 | 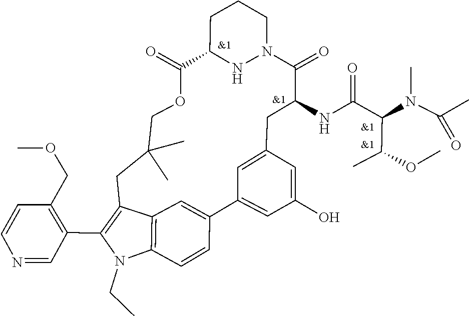 [SMILES: CCn1c(-c2cnccc2COC)c2c3cc(ccc31)-c1cc(O)cc(c1)C[C@H](NC(=O)[C@H]([C@@H](C)OC)N(C)C(C)=O)C(=O)N1CCC[C@H](N1)C(=O)OCC(C)(C)C2] |
| AA77 | 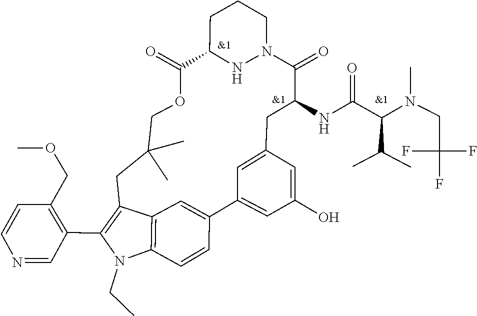 [SMILES: CCn1c(-c2cnccc2COC)c2c3cc(ccc31)-c1cc(O)cc(c1)C[C@H](NC(=O)[C@H](C(C)C)N(C)CC(F)(F)F)C(=O)N1CCC[C@H](N1)C(=O)OCC(C)(C)C2] |
| AA78 | 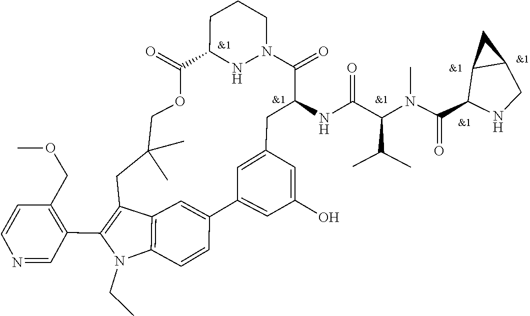 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NC[C@@H]3C[C@@H]31)C(=O)N1CCC[C@H](N1)C(=O)OCC(C)(C)C2] |
| AA79 | 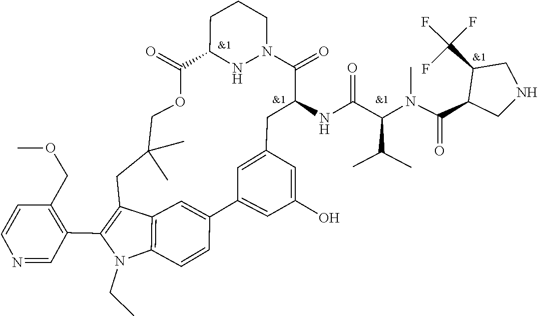 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1C(F)(F)F)C(=O)N1CCC[C@H](N1)C(=O)OCC(C)(C)C2] |
| AA80 | 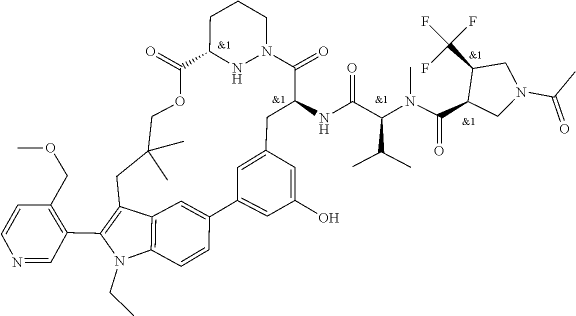 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CN(C(C)=O)C[C@@H]1C(F)(F)F)C(=O)N1CCC[C@H](N1)C(=O)OCC(C)(C)C2] |
| AA81 | 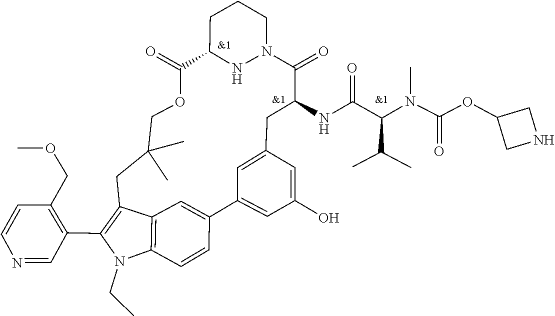 [SMILES: CCn1c(-c2cnccc2COC)c2c3cc(ccc31)-c1cc(O)cc(c1)C[C@H](NC(=O)[C@H](C(C)C)N(C)C(=O)OC1CNC1)C(=O)N1CCC[C@H](N1)C(=O)OCC(C)(C)C2] |
| AA82 | 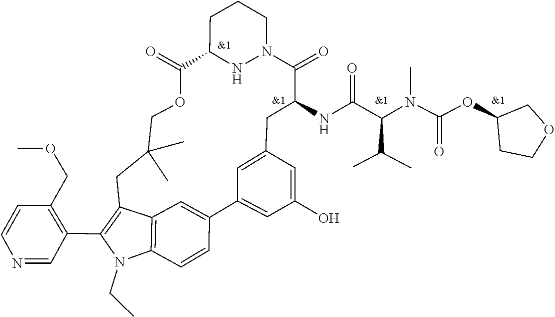 [SMILES: CCn1c(-c2cnccc2COC)c2c3cc(ccc31)-c1cc(O)cc(c1)C[C@H](NC(=O)[C@H](C(C)C)N(C)C(=O)O[C@@H]1CCOC1)C(=O)N1CCC[C@H](N1)C(=O)OCC(C)(C)C2] |
| AA83 | 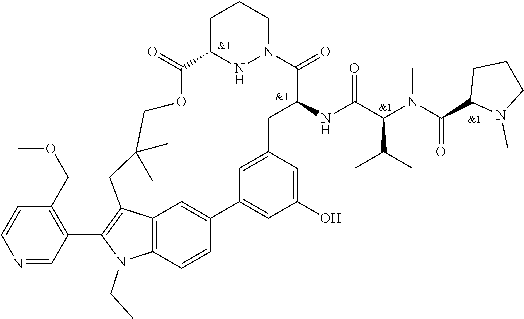 |
| AA84 | 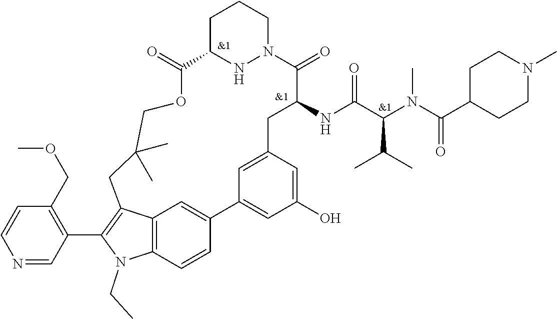 [SMILES: CCn1c(-c2cnccc2COC)c2c3cc(ccc31)-c1cc(O)cc(c1)C[C@H](NC(=O)[C@H](C(C)C)N(C)C(=O)C1CCN(C)CC1)C(=O)N1CCC[C@H](N1)C(=O)OCC(C)(C)C2] |
| AA85 | 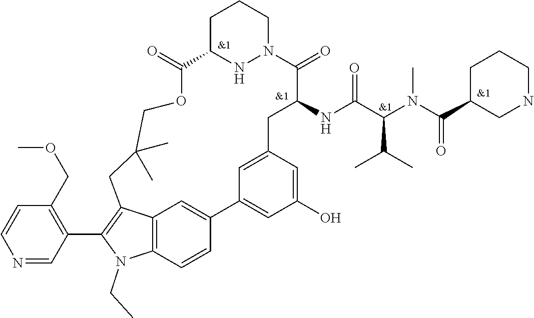 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCNC1)C(=O)N1CCC[C@H](N1)C(=O)OCC(C)(C)C2] |
| AA86 | 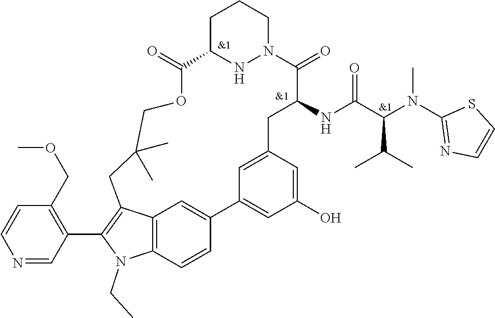 [SMILES: CCn1c(-c2cnccc2COC)c2c3cc(ccc31)-c1cc(O)cc(c1)C[C@H](NC(=O)[C@H](C(C)C)N(C)c1nccs1)C(=O)N1CCC[C@H](N1)C(=O)OCC(C)(C)C2] |
| AA87 | 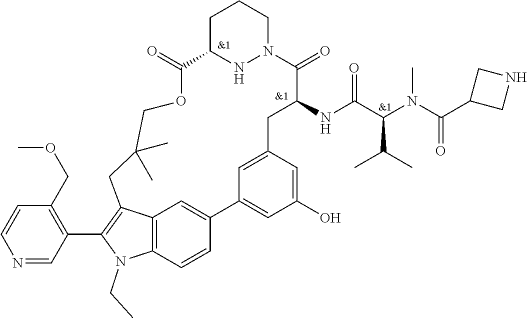 [SMILES: CCn1c(-c2cnccc2COC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| AA88 | 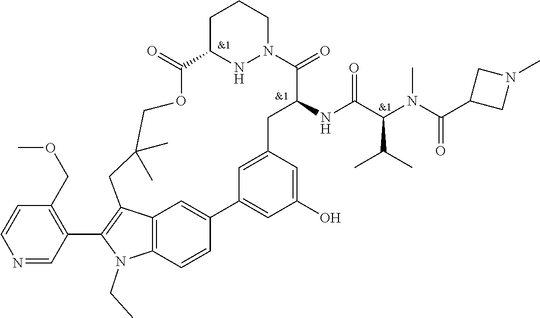 [SMILES: CCn1c(-c2cnccc2COC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| AA89 | 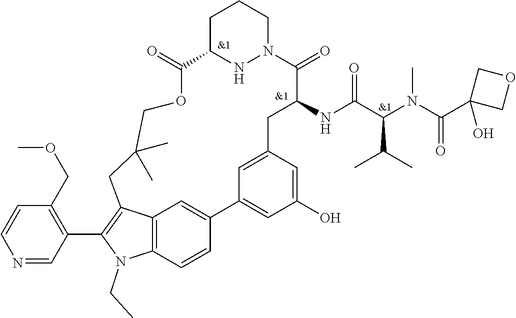 [SMILES: CCn1c(-c2cnccc2COC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| AA90 | 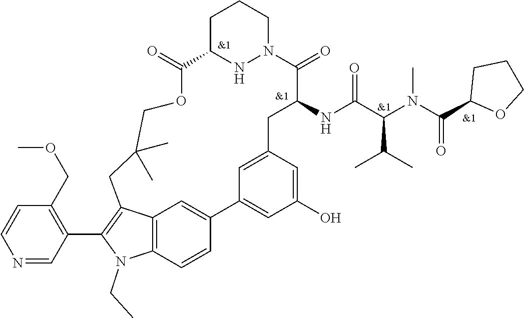 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCCO1)C(=O)N1CCC[C@H](N1)C(=O)OCC(C)(C)C2] |
| AA91 | 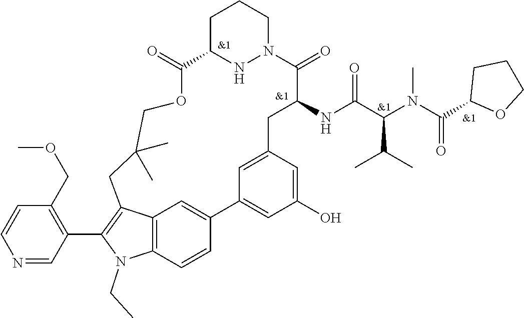 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCO1)C(=O)N1CCC[C@H](N1)C(=O)OCC(C)(C)C2] |
| AA92 | 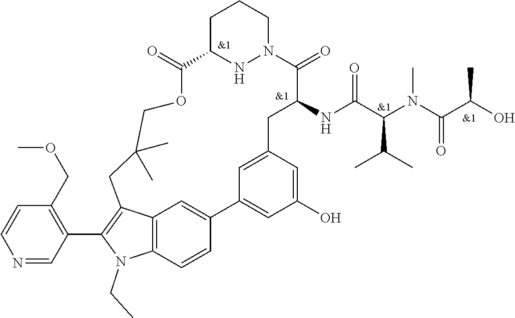 [SMILES: CCn1c(-c2cnccc2COC)c2c3cc(ccc31)-c1cc(O)cc(c1)C[C@H](NC(=O)[C@H](C(C)C)N(C)C(=O)[C@@H](C)O)C(=O)N1CCC[C@H](N1)C(=O)OCC(C)(C)C2] |
| AA93 | 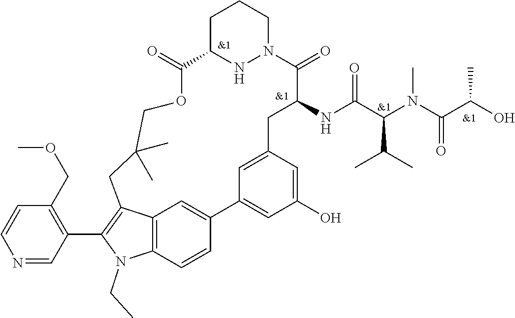 [SMILES: CCn1c(-c2cnccc2COC)c2c3cc(ccc31)-c1cc(O)cc(c1)C[C@H](NC(=O)[C@H](C(C)C)N(C)C(=O)[C@H](C)O)C(=O)N1CCC[C@H](N1)C(=O)OCC(C)(C)C2] |
| AA94 | 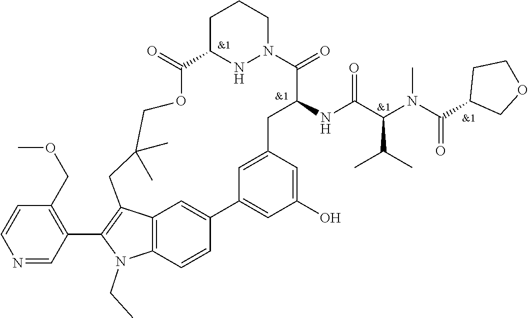 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCOC1)C(=O)N1CCC[C@H](N1)C(=O)OCC(C)(C)C2] |
| AA95 | 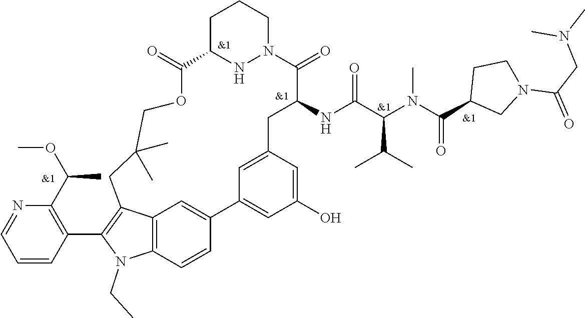 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA96 | 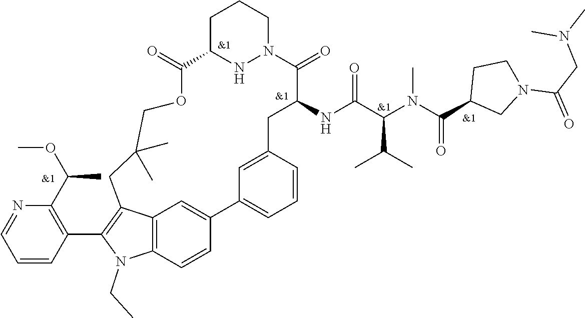 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA97 | 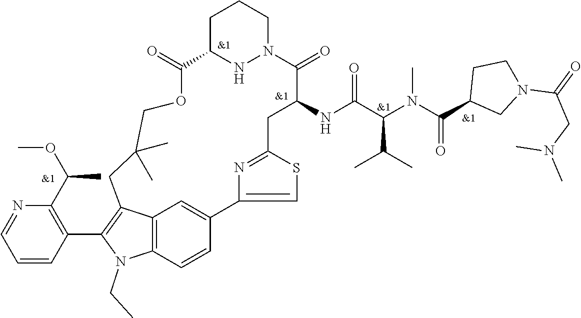 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN(C)C)C1)C(=O)N1CCC[C@H](N1)C(=O)OCC(C)(C)C2] |
| AA98 | 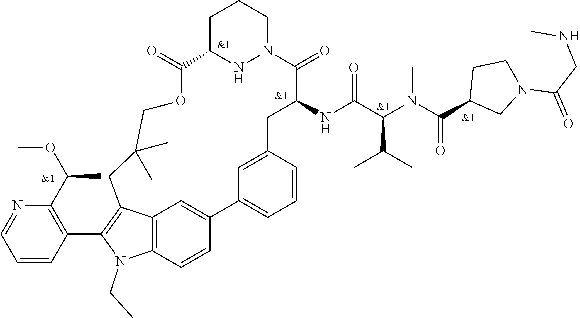 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA99 | 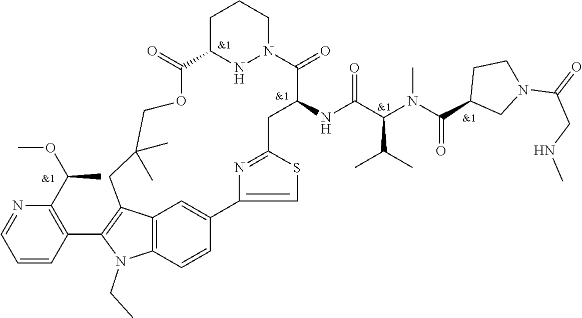 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA100 | 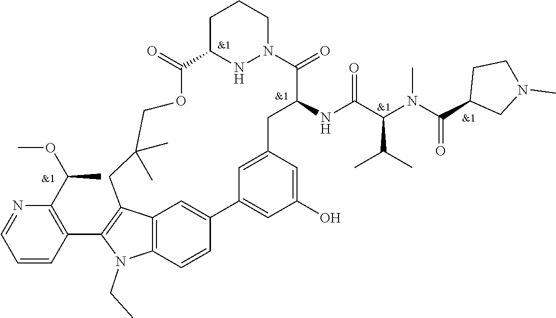 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA101 | 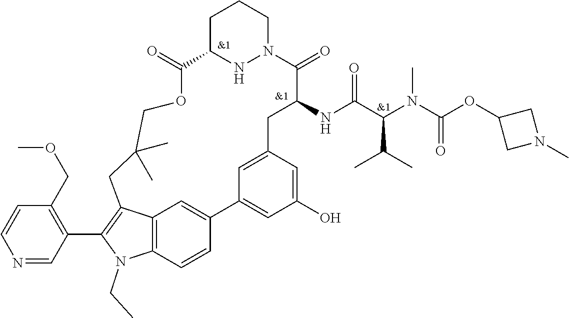 [SMILES: CCn1c(-c2cnccc2COC)c2c3cc(ccc31)-c1cc(O)cc(c1)C[C@H](NC(=O)[C@H](C(C)C)N(C)C(=O)OC1CN(C)C1)C(=O)N1CCC[C@H](N1)C(=O)OCC(C)(C)C2] |
| AA102 | 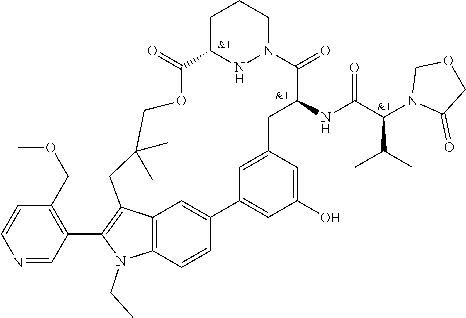 [SMILES: CCn1c(-c2cnccc2COC)c2c3cc(ccc31)-c1cc(O)cc(c1)C[C@H](NC(=O)[C@H](C(C)C)N1COCC1=O)C(=O)N1CCC[C@H](N1)C(=O)OCC(C)(C)C2] |
| AA103 | 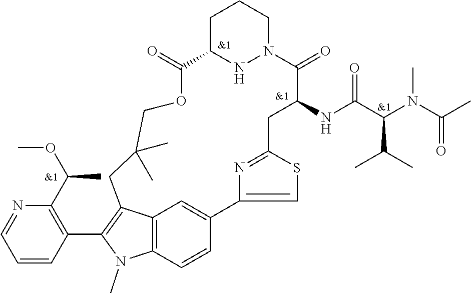 [SMILES: CO[C@@H](C)c1ncccc1-c1c2c3cc(ccc3n1C)-c1csc(n1)C[C@H](NC(=O)[C@H](C(C)C)N(C)C(C)=O)C(=O)N1CCC[C@H](N1)C(=O)OCC(C)(C)C2] |
| AA104 | 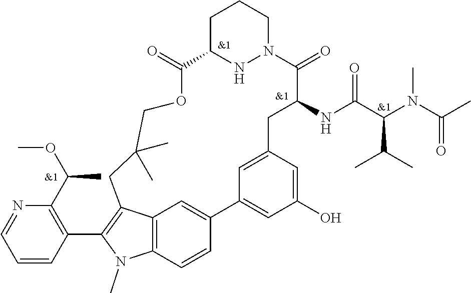 [SMILES: CO[C@@H](C)c1ncccc1-c1c2c3cc(ccc3n1C)-c1cc(O)cc(c1)C[C@H](NC(=O)[C@H](C(C)C)N(C)C(C)=O)C(=O)N1CCC[C@H](N1)C(=O)OCC(C)(C)C2] |
| AA105 | 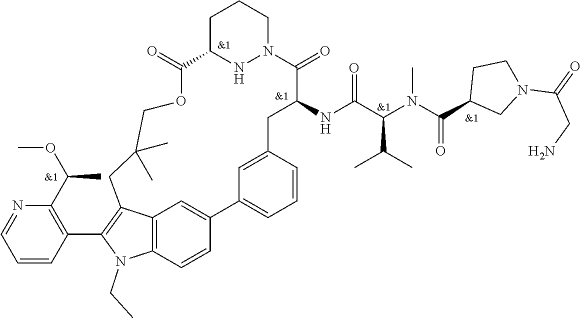 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| AA106 | 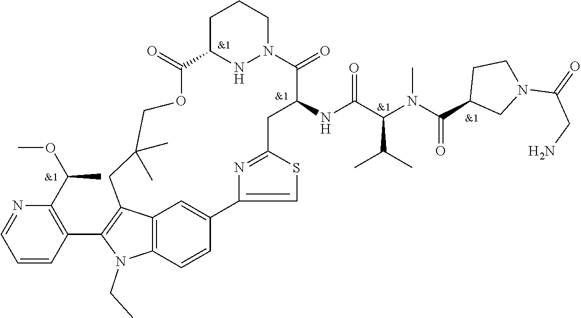 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)CN)C1)C(=O)N1CCC[C@H](N1)C(=O)OCC(C)(C)C2] |
| AA107 | 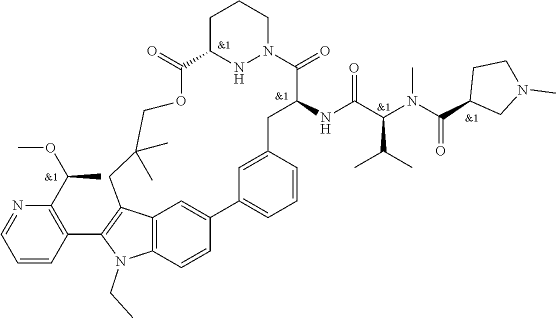 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C)C1)C(=O)N1CCC[C@H](N1)C(=O)OCC(C)(C)C2] |
| AA108 | 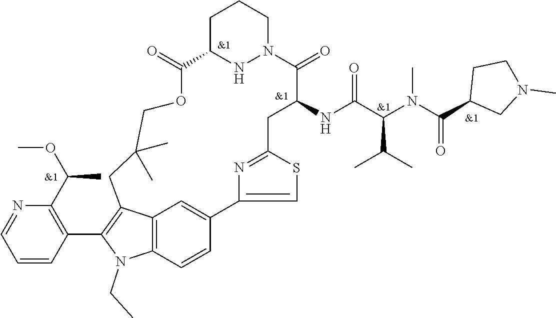 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C)C1)C(=O)N1CCC[C@H](N1)C(=O)OCC(C)(C)C2] |
| AA109 | 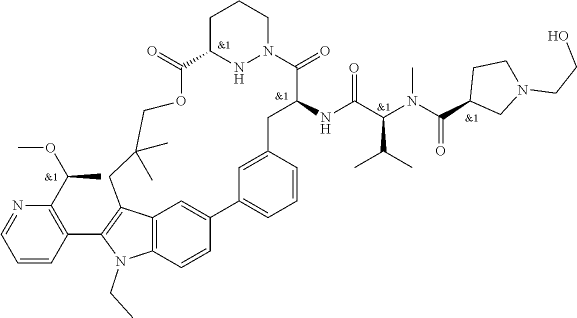 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA110 | 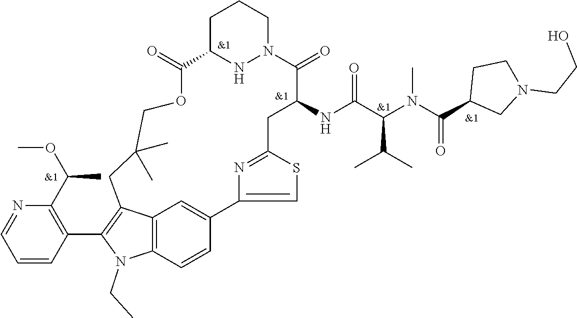 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA111 | 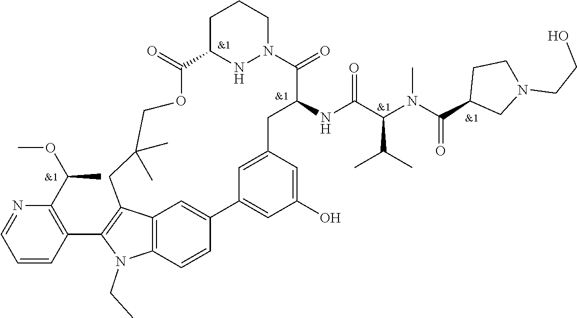 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(CCO)C1)C(=O)N1CCC[C@H](N1)C(=O)OCC(C)(C)C2] |
| AA112 | 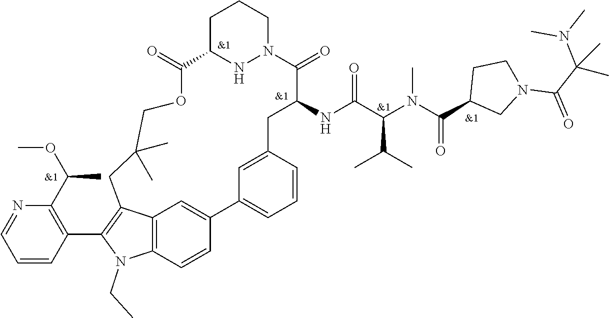 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA113 | 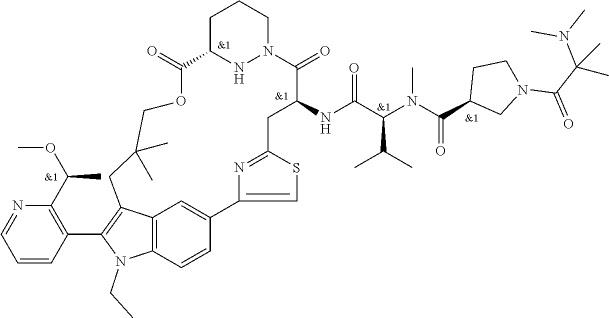 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA114 | 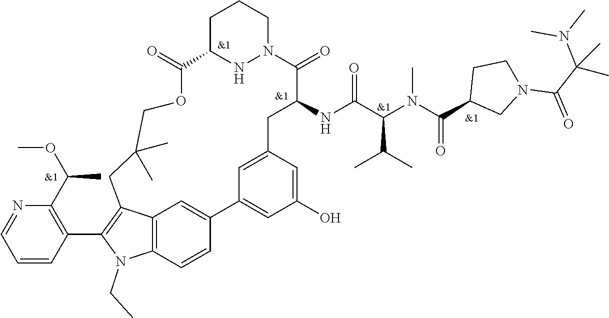 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C(C)(C)N(C)C)C1)C(=O)N1CCC[C@H](N1)C(=O)OCC(C)(C)C2] |
| AA115 | 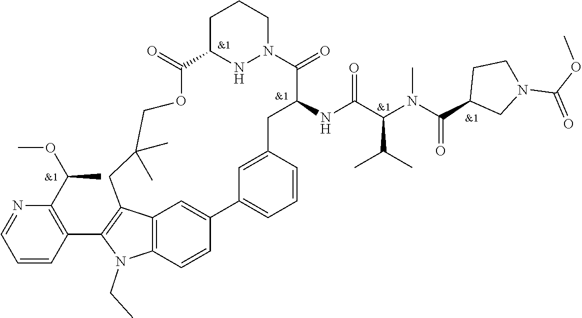 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| AA116 | 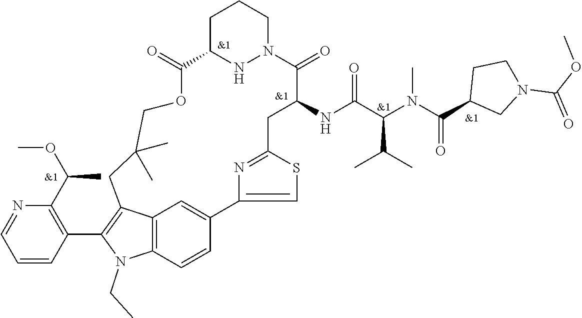 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCN(C(=O)OC)C1)C(=O)N1CCC[C@H](N1)C(=O)OCC(C)(C)C2] |
| AA117 | 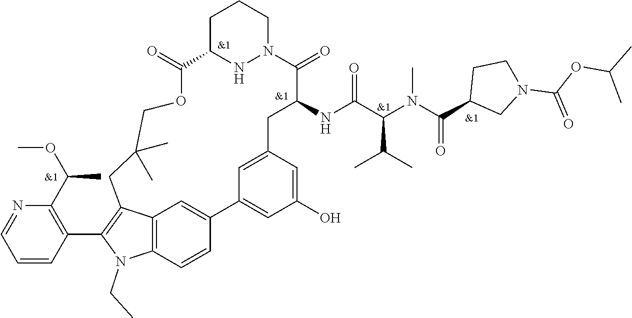 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)OC(C)C)C1)C(=O)N1CCC[C@H](N1)C(=O)OCC(C)(C)C2] |
| AA118 | 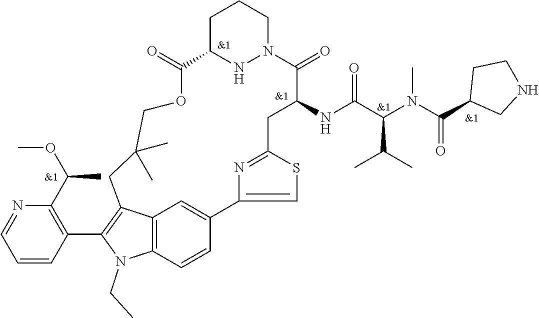 [SMILES: CCn1c(-c2cccnc2[C@H](C)OC)c2c3cc(ccc31)-c1csc(n1)C[C@H](NC(=O)[C@H](C(C)C)N(C)C(=O)[C@H]1CCNC1)C(=O)N1CCC[C@H](N1)C(=O)OCC(C)(C)C2] |
| AA119 | 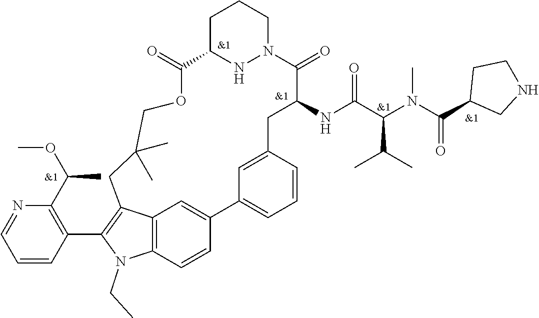 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCNC1)C(=O)N1CCC[C@H](N1)C(=O)OCC(C)(C)C2] |
| AA120 | 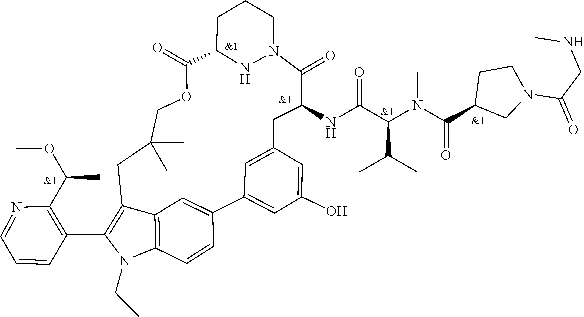 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)CNC)C1)C(=O)N1CCC[C@H](N1)C(=O)OCC(C)(C)C2] |
| AA121 | 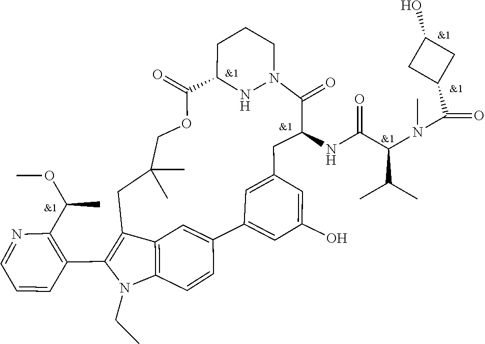 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H](O)C1)C(=O)N1CCC[C@H](N1)C(=O)OCC(C)(C)C2] |
| AA122 | 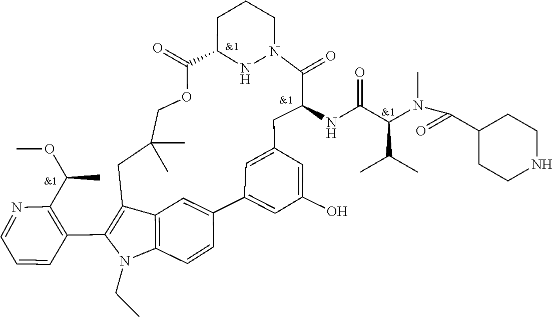 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNCC1)C(=O)N1CCC[C@H](N1)C(=O)OCC(C)(C)C2] |
| AA123 | 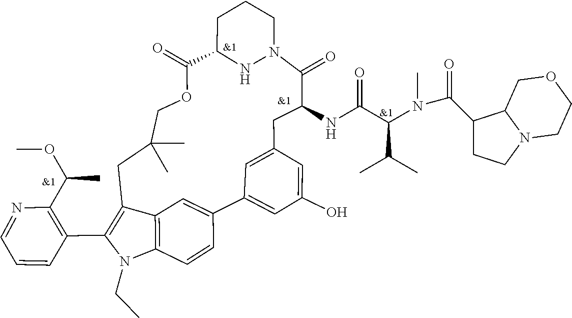 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCN3CCOCC13)C(=O)N1CCC[C@H](N1)C(=O)OCC(C)(C)C2] |
| AA124 | 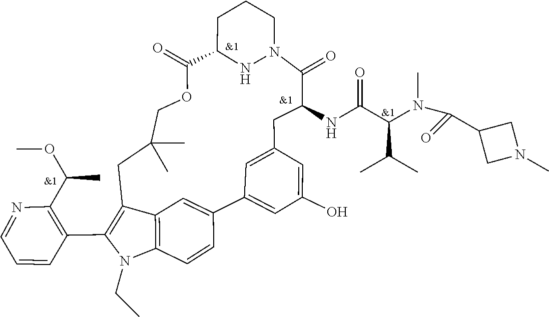 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CN(C)C1)C(=O)N1CCC[C@H](N1)C(=O)OCC(C)(C)C2] |
| AA125 | 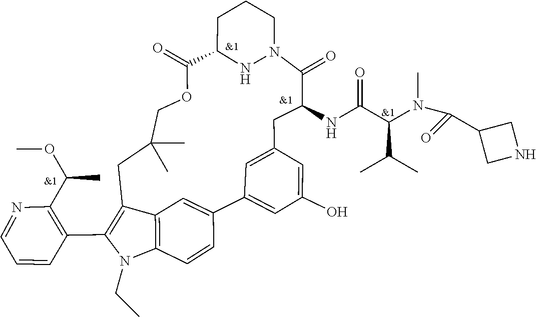 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CNC1)C(=O)N1CCC[C@H](N1)C(=O)OCC(C)(C)C2] |
| AA126 | 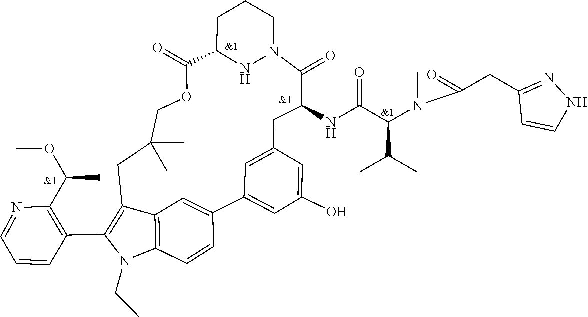 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1cc[nH]n1)C(=O)N1CCC[C@H](N1)C(=O)OCC(C)(C)C2] |
| AA127 | 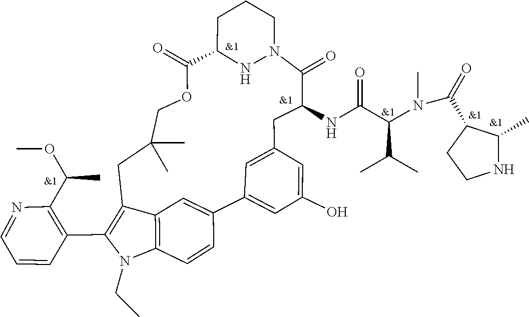 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN[C@H]1C)C(=O)N1CCC[C@H](N1)C(=O)OCC(C)(C)C2] |
| AA128 | 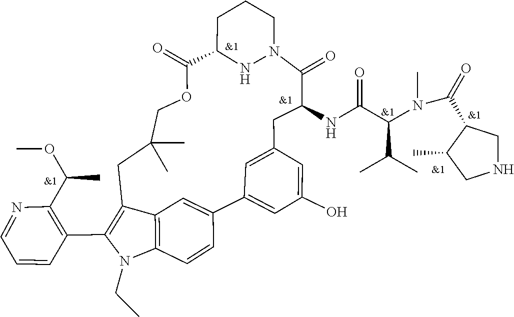 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1C)C(=O)N1CCC[C@H](N1)C(=O)OCC(C)(C)C2] |
| AA129 | 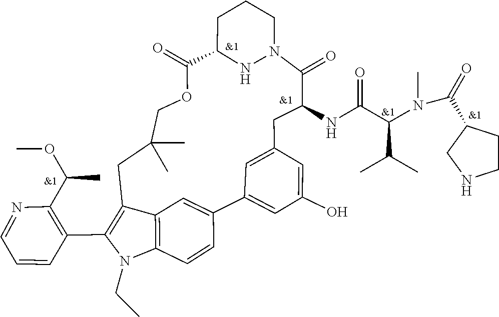 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CCNC1)C(=O)N1CCC[C@H](N1)C(=O)OCC(C)(C)C2] |
| AA130 | 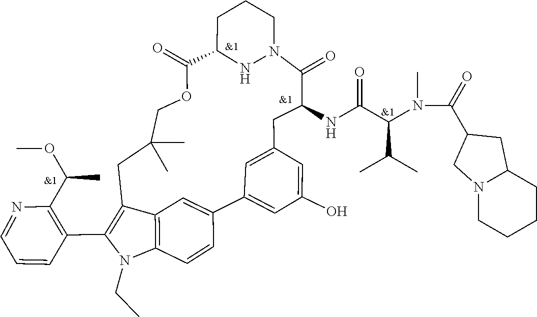 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CCCCN3C1)C(=O)N1CCC[C@H](N1)C(=O)OCC(C)(C)C2] |
| AA131 | 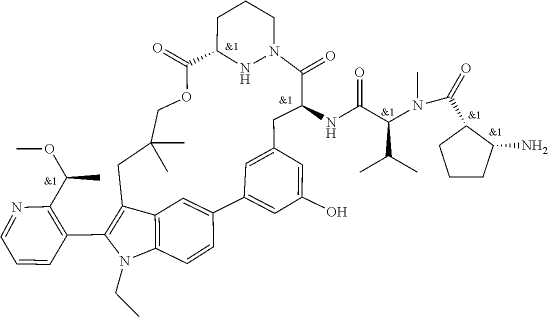 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCC[C@H]1N)C(=O)N1CCC[C@H](N1)C(=O)OCC(C)(C)C2] |
| AA132 | 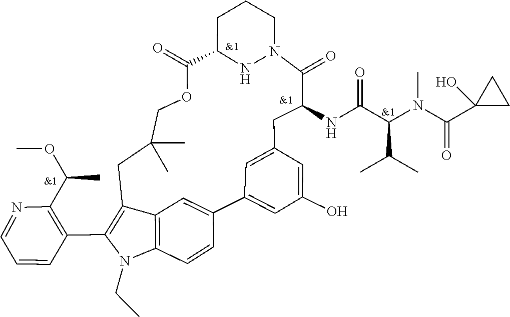 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)CC1)C(=O)N1CCC[C@H](N1)C(=O)OCC(C)(C)C2] |
| AA133 | 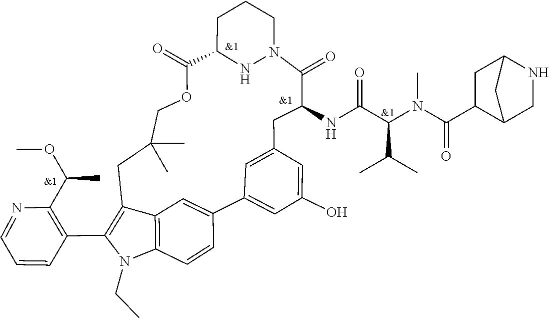 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC3CC1CN3)C(=O)N1CCC[C@H](N1)C(=O)OCC(C)(C)C2] |
| AA134 | 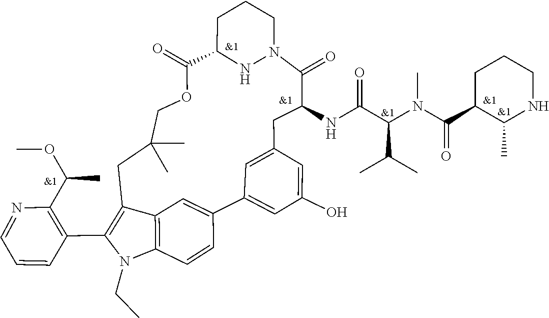 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCCN[C@@H]1C)C(=O)N1CCC[C@H](N1)C(=O)OCC(C)(C)C2] |
| AA135 | 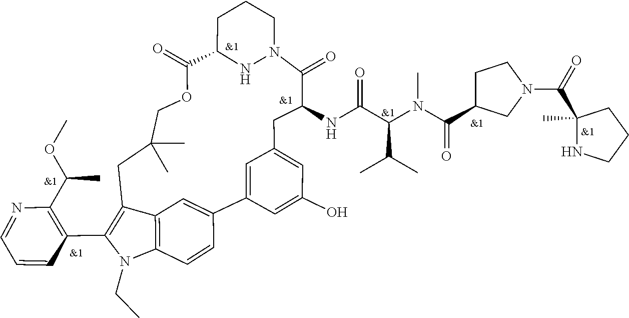 |
| AA136 | 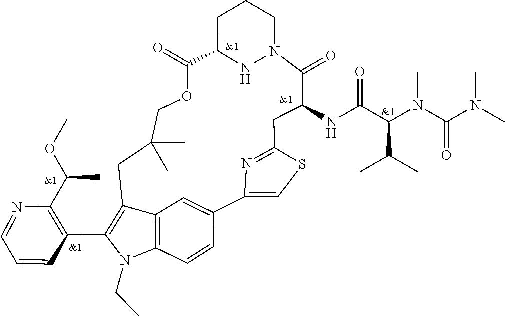 |
| AA137 | 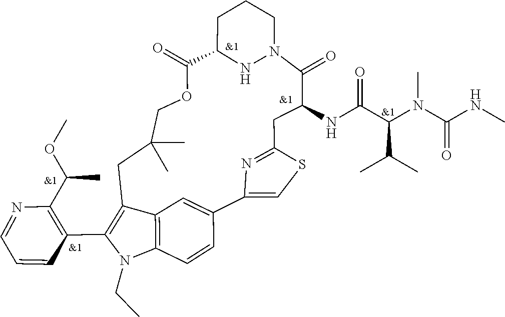 |
| AA138 | 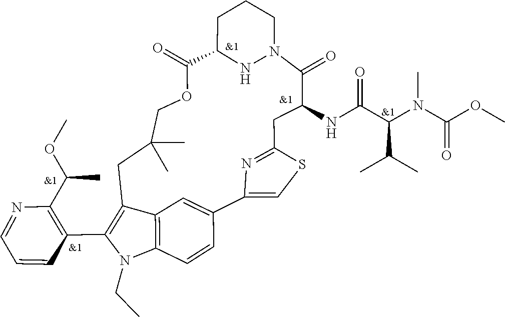 |
| AA139 | 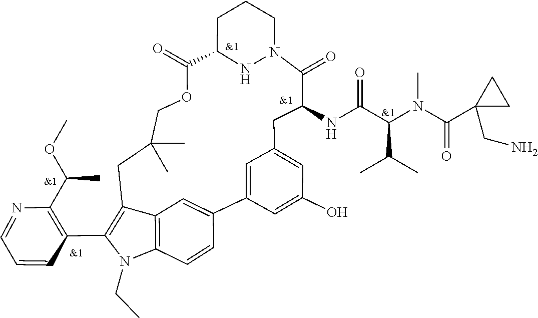 |
| AA140 | 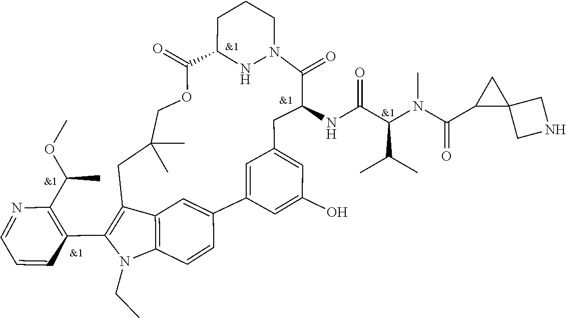 |
| AA141 | 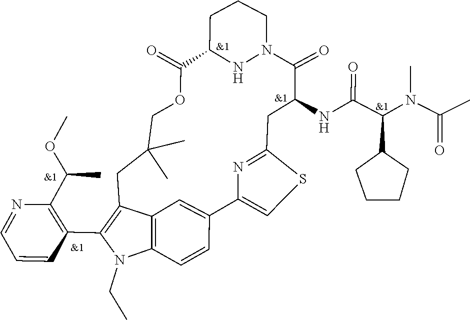 |
| AA142 | 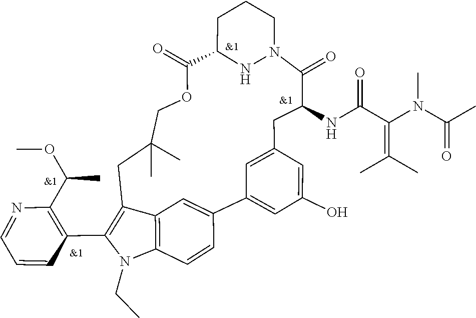 |
| AA143 | 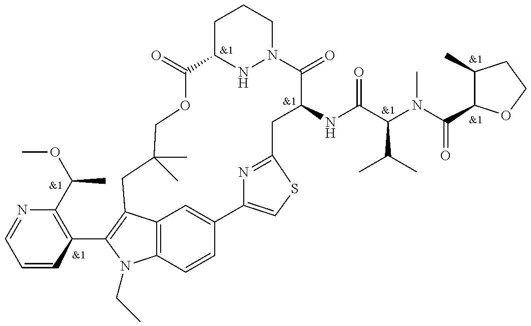 |
| AA144 | 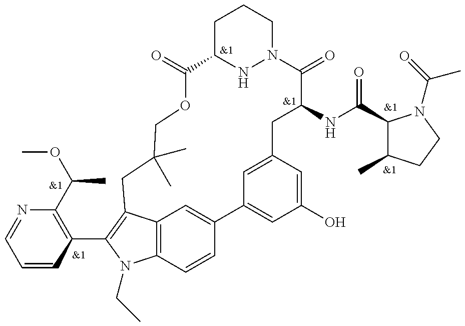 |
| AA145 | 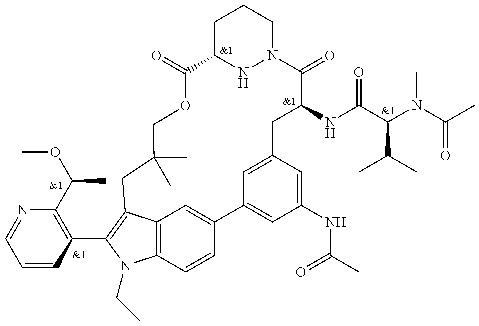 |
| AA146 | 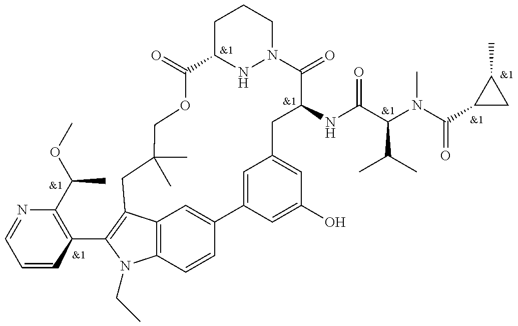 |
| AA147 | 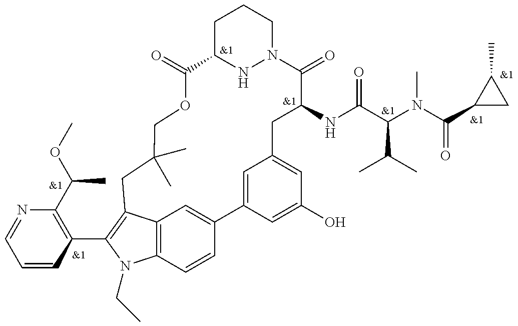 |
| AA148 | 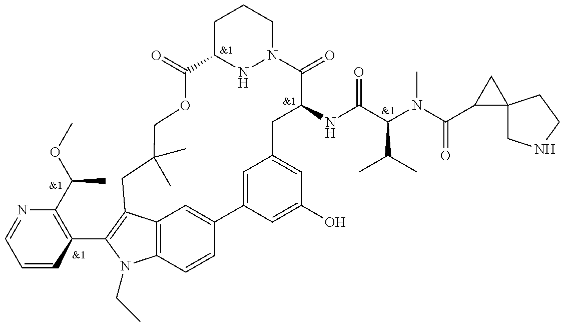 |
| AA149 | 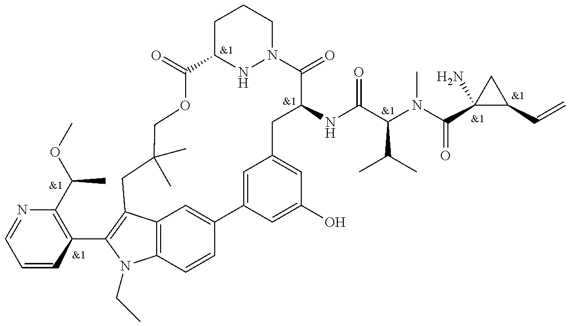 |
| AA150 | 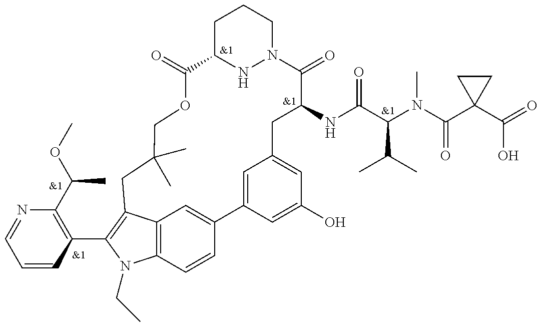 |
| AA151 | 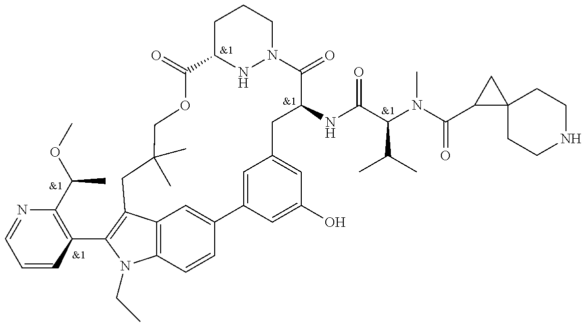 |
| AA152 | 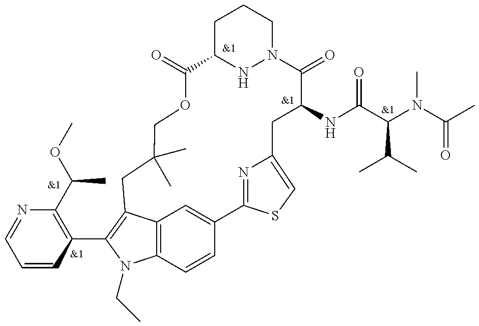 |
| AA153 | 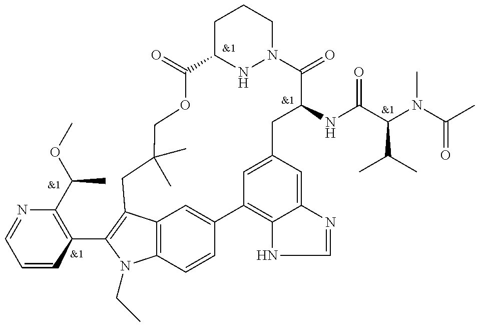 |
| AA154 | 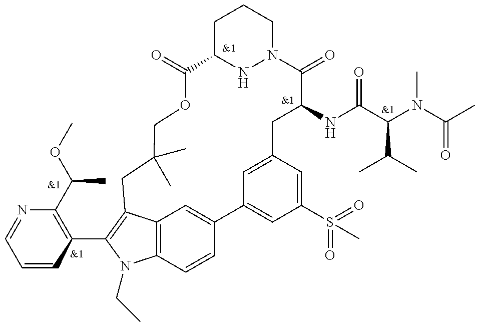 |
| AA155 | 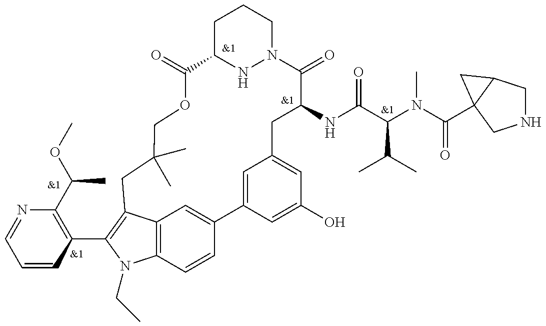 |
| AA156 | 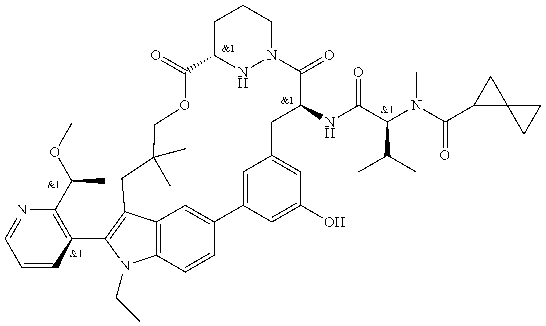 |
| AA157 | 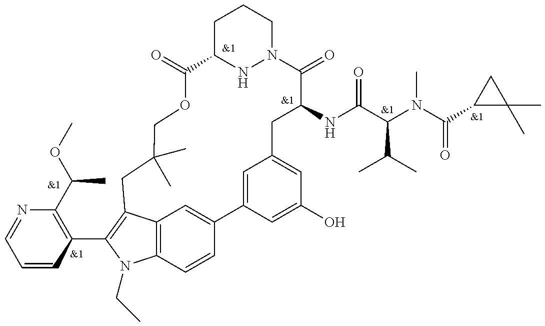 |
| AA158 | 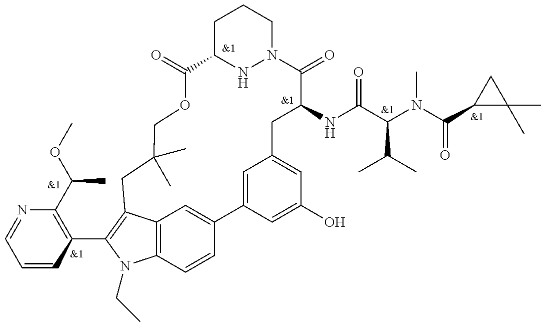 |
| AA159 | 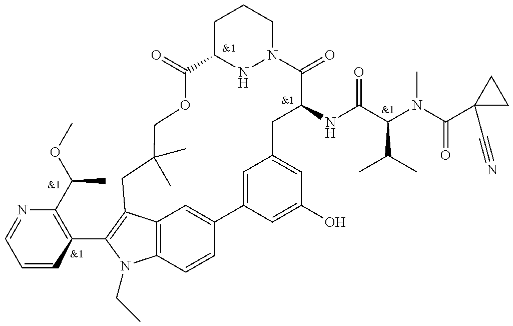 |
| AA160 | 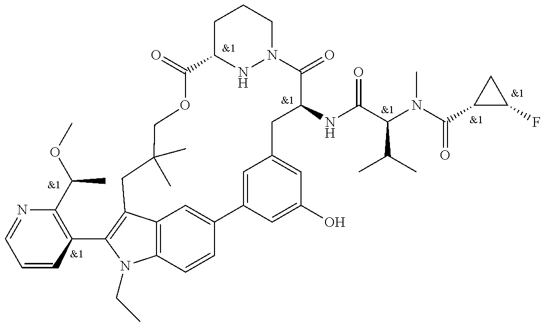 |
| AA161 | 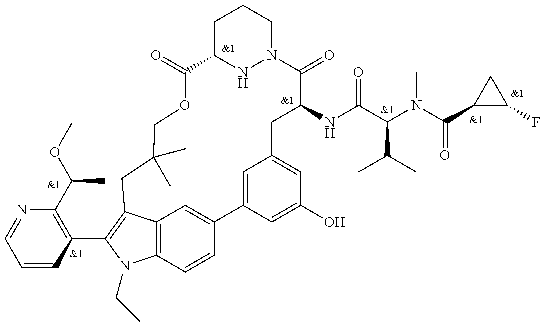 |
| AA162 | 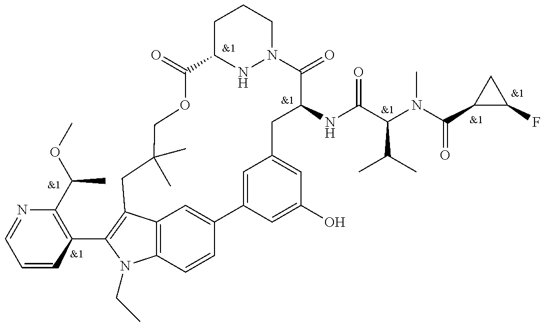 |
| AA163 | 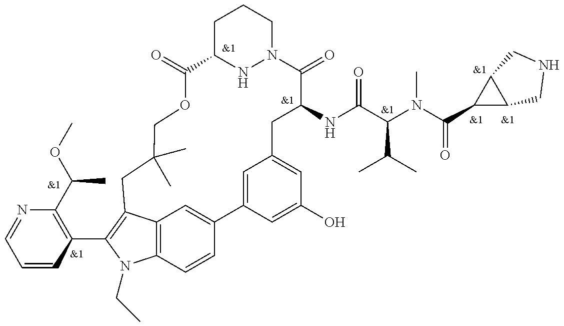 |
| AA164 | 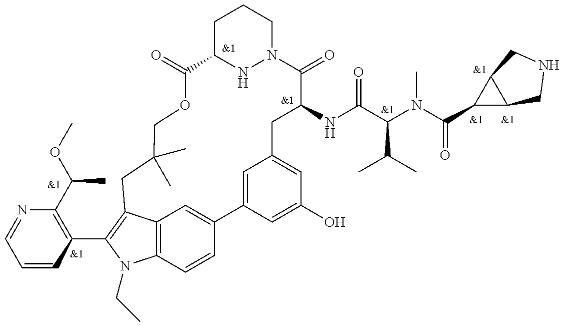 |
| AA165 | 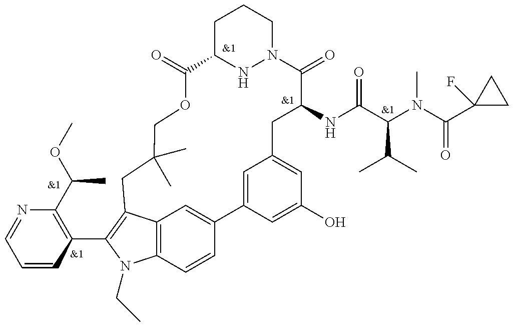 |
| AA166 | 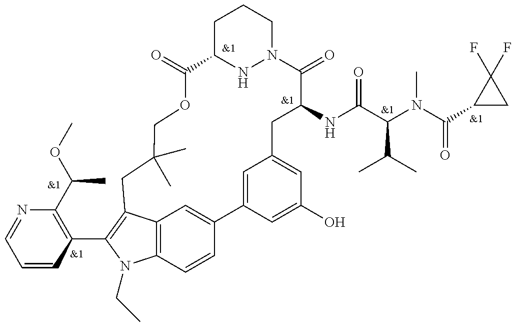 |
| AA167 | 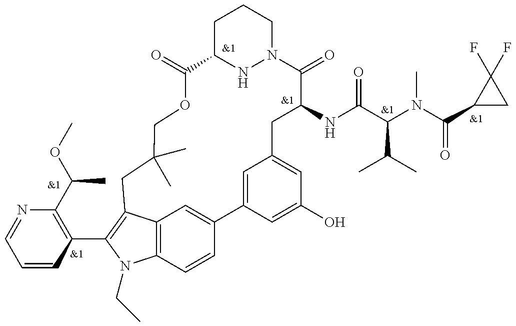 |
| AA168 | 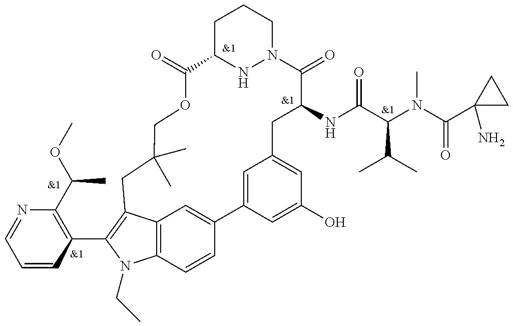 |
| AA169 | 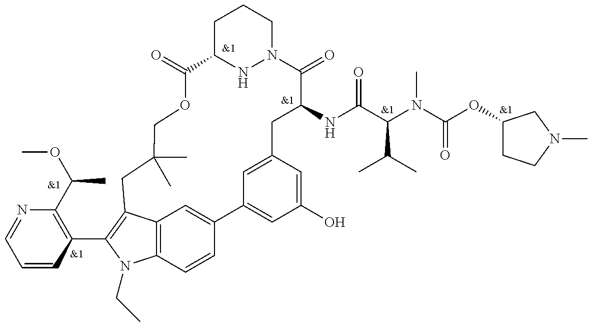 |
| AA170 | 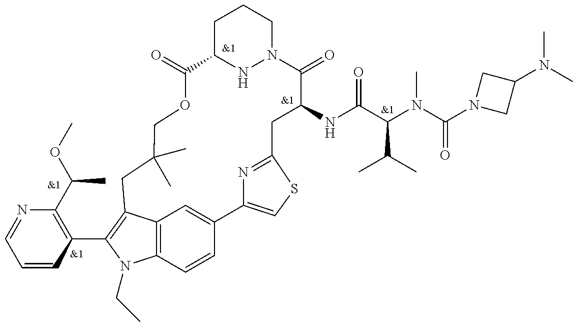 |
| AA171 | 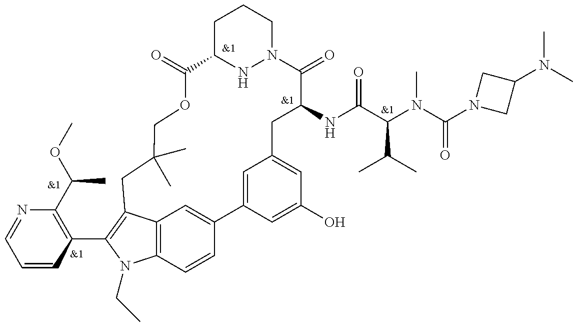 |
| AA172 | 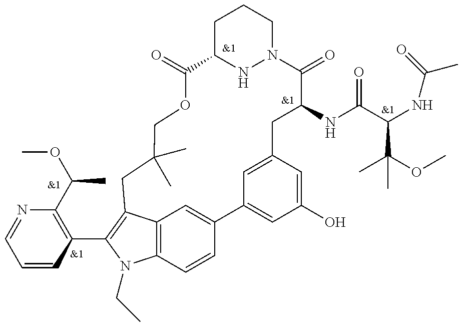 |
| AA173 | 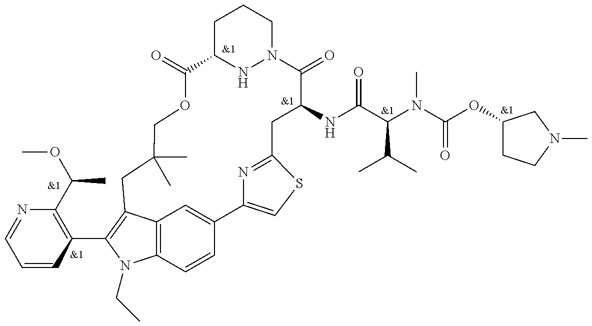 |
| AA174 | 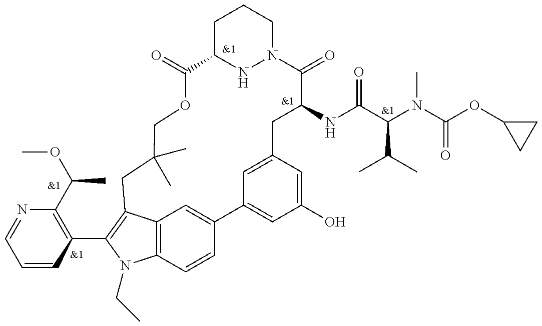 |
| AA175 | 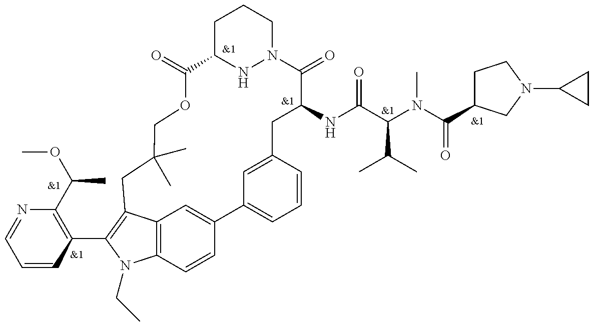 |
| AA176 | 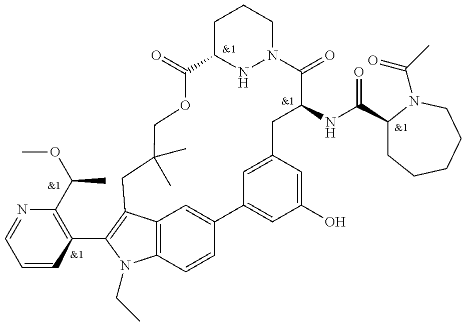 |
| AA177 | 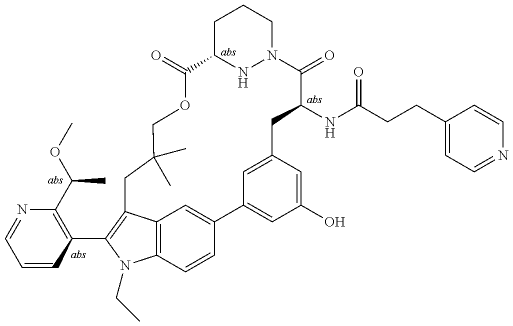 |
| AA178 | 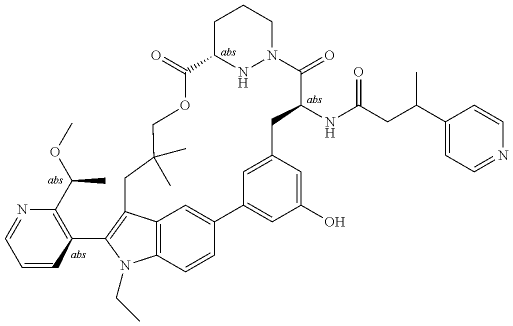 |
| AA179 | 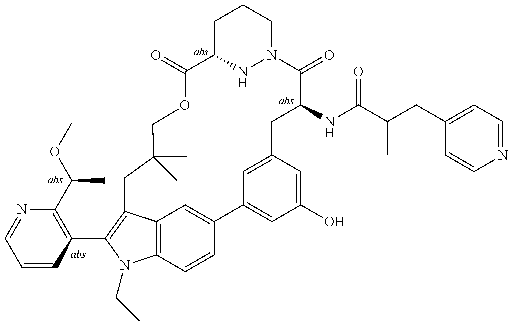 |
| AA180 | 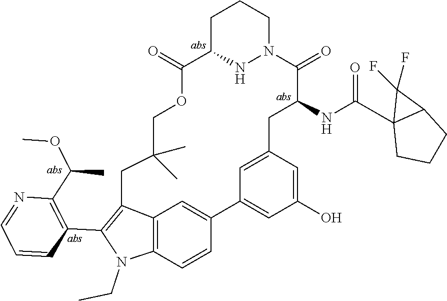 |
| AA181 | 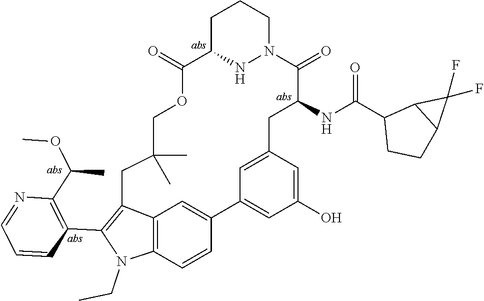 |
| AA182 | 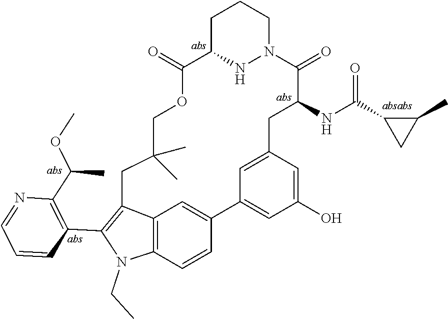 |
| AA183 | 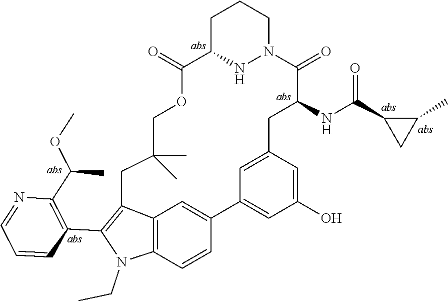 |
| AA184 | 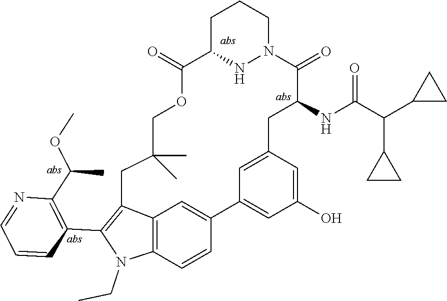 |
| AA185 | 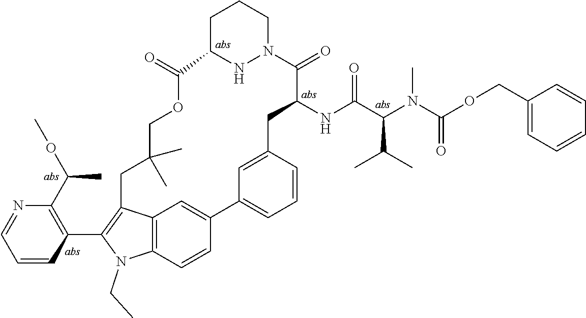 |
| AA186 | 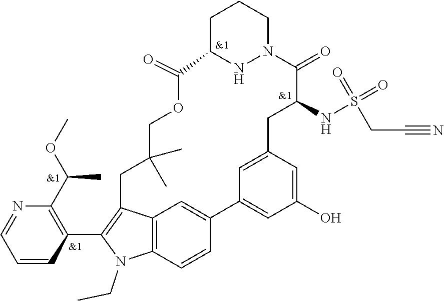 |
| AA187 | 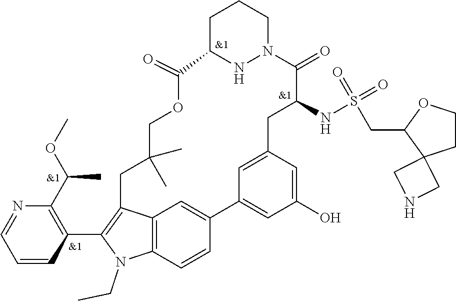 |
| AA188 | 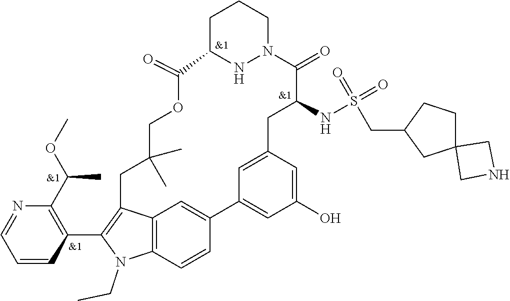 |
| AA189 | 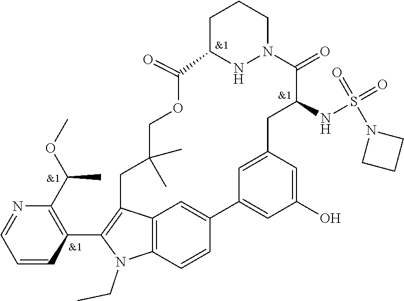 |
| AA190 | 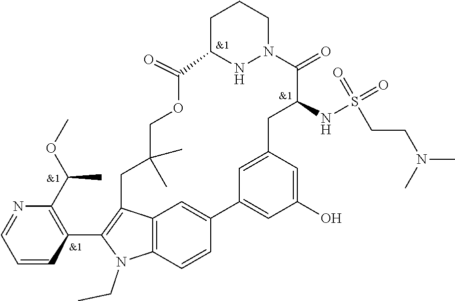 |
| AA191 | 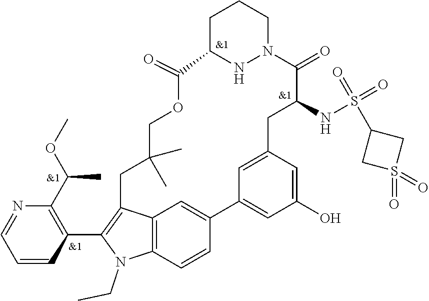 |
| AA192 | 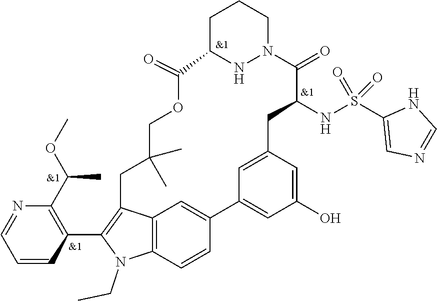 |
| AA193 | 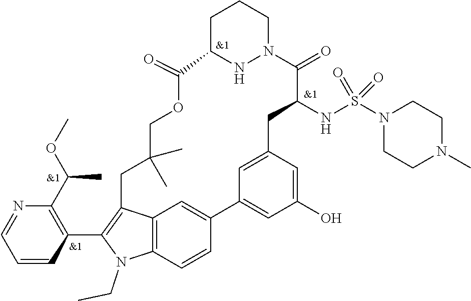 |
| AA194 | 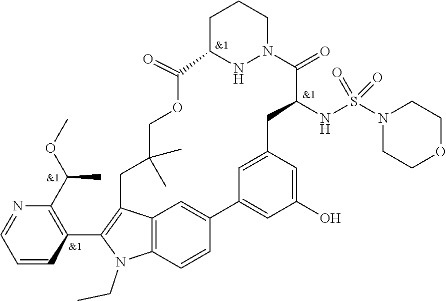 |
| AA195 | 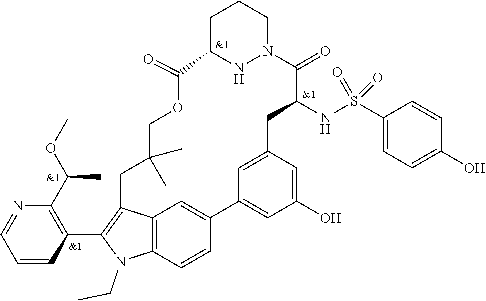 |
| AA196 | 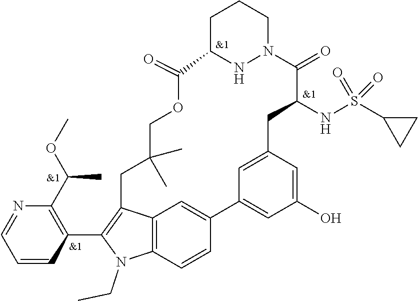 |
| AA197 | 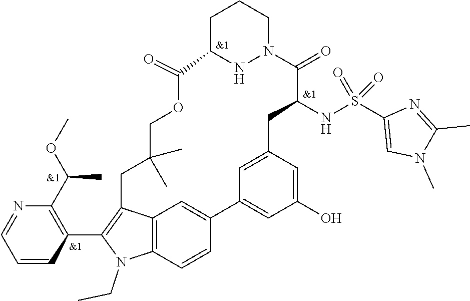 |
| AA198 | 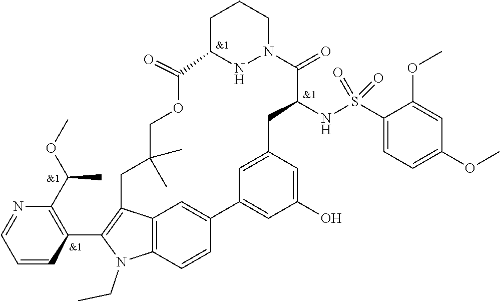 |
| AA199 | 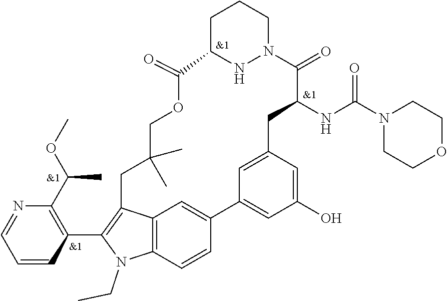 |
| AA200 | 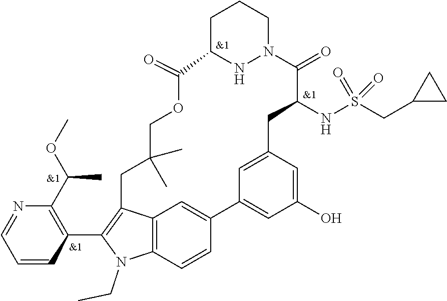 |
| AA201 | 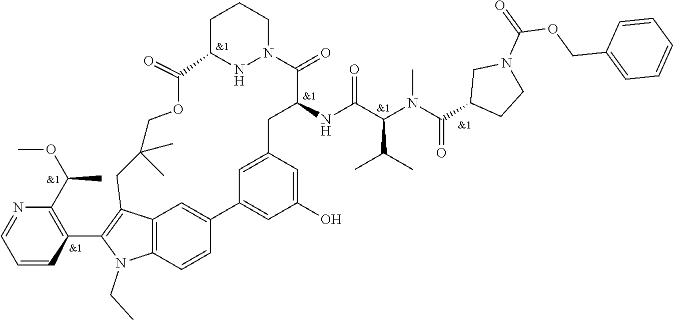 |
| AA202 | 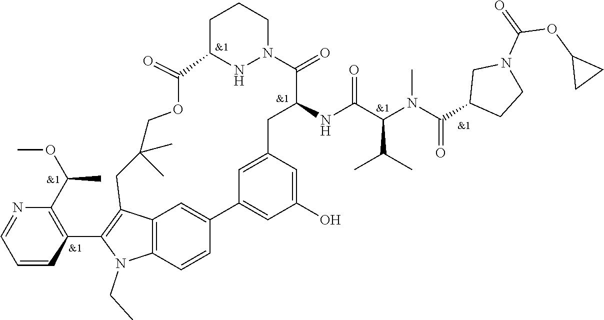 |
| AA203 | 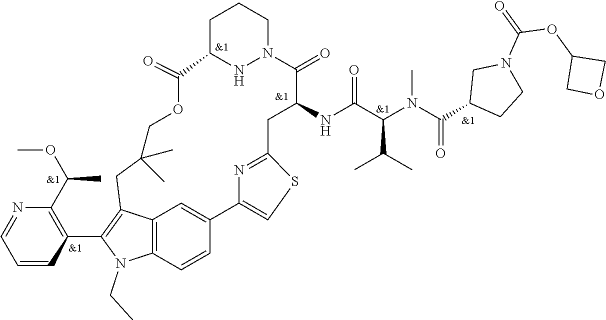 |
| AA204 | 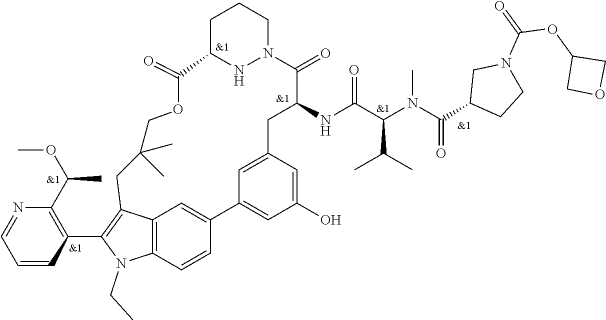 |
| AA205 | 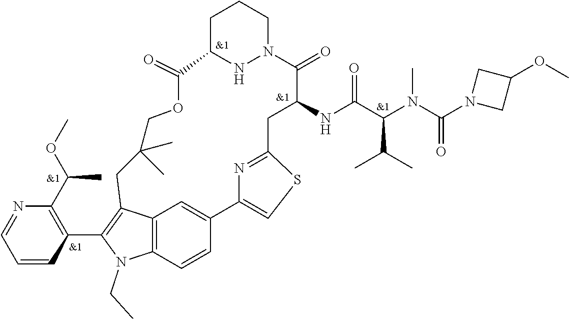 |
| AA206 | 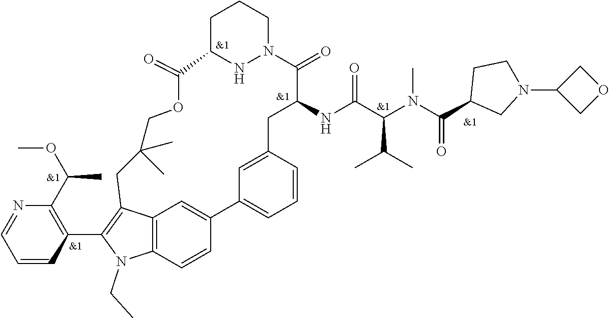 |
| AA207 | 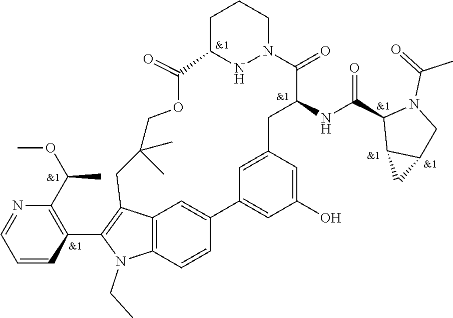 |
| AA208 | 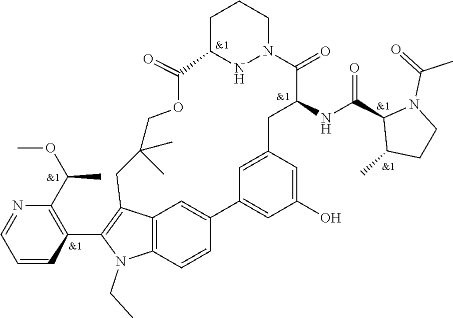 |
| AA209 | 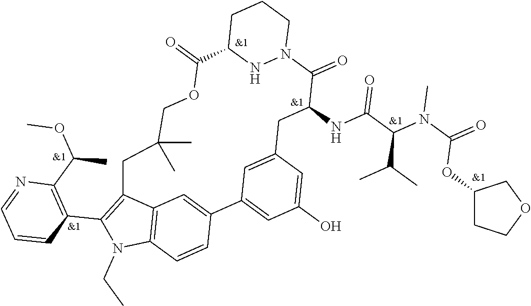 |
| AA210 | 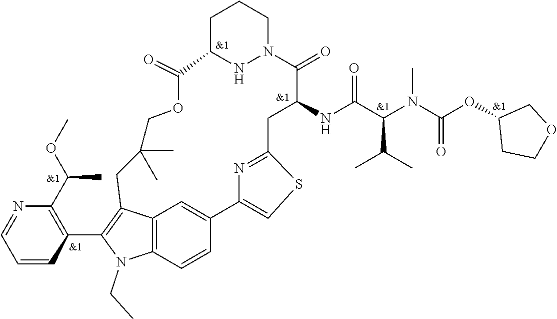 |
| AA211 | 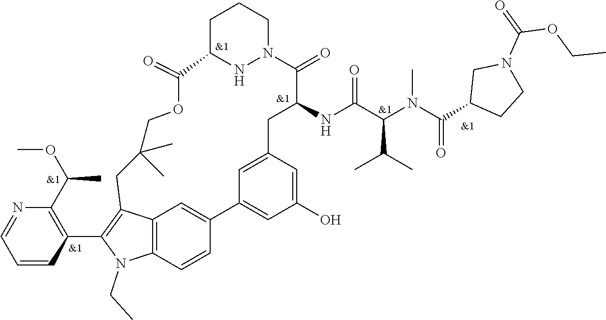 |
| AA212 | 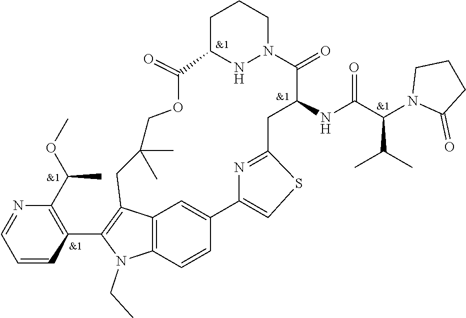 |
| AA213 | 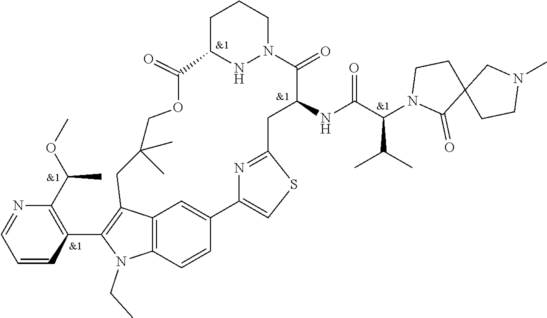 |
| AA214 | 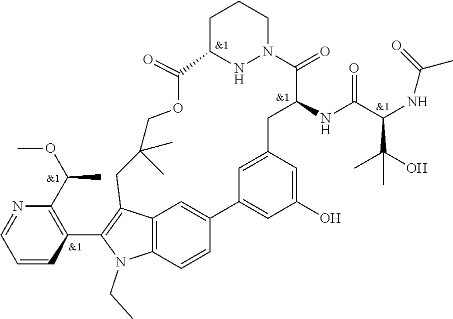 |
| AA215 | 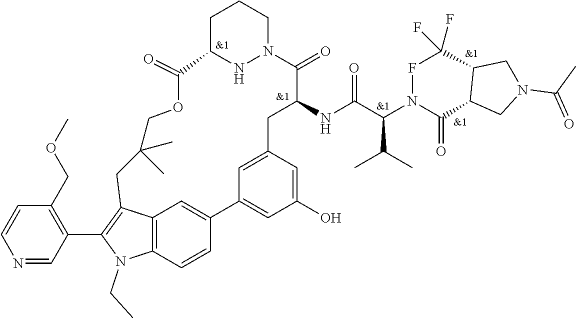 |
| AA216 | 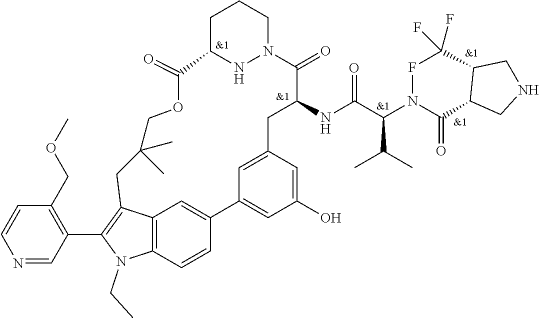 |
| AA217 | 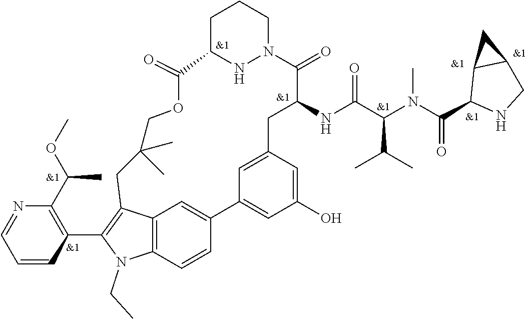 |
| AA218 | 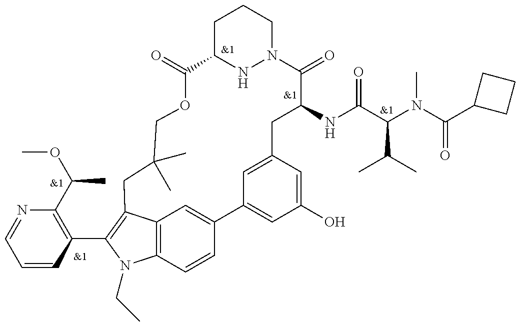 |
| AA219 | 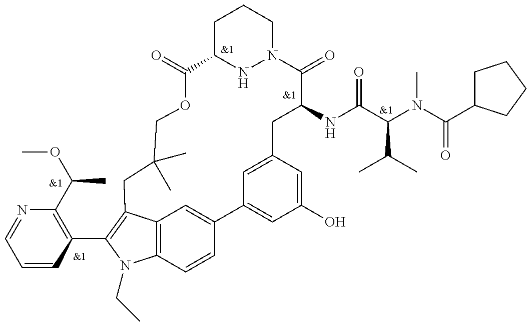 |
| AA220 | 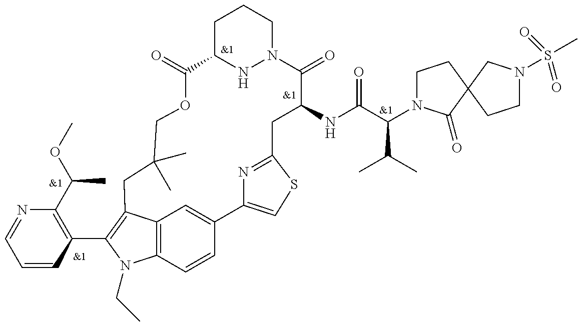 |
| AA221 | 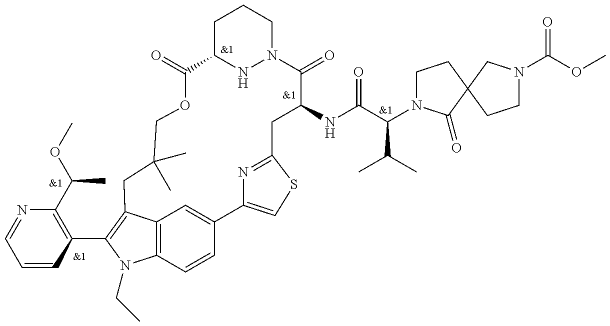 |
| AA222 | 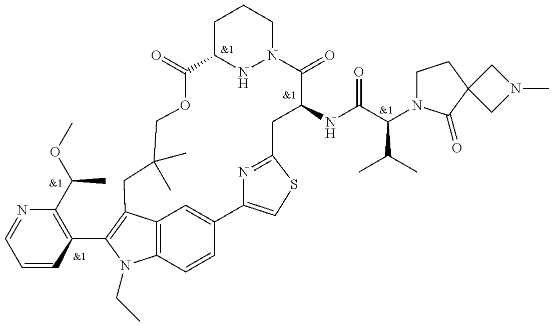 |
| AA223 | 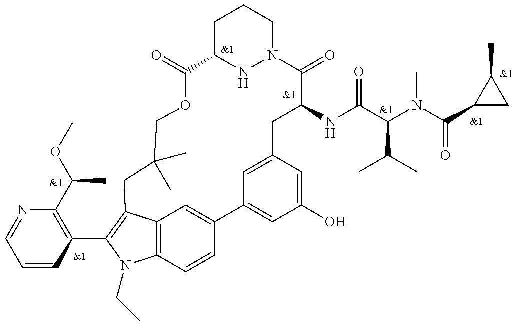 |
| AA224 | 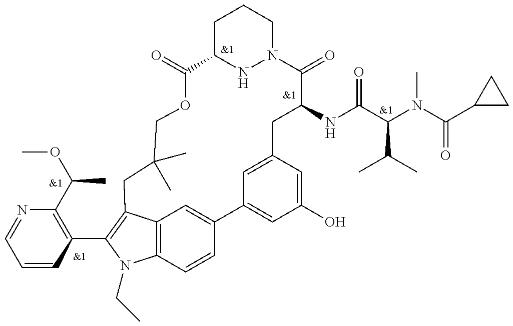 |
| AA225 | 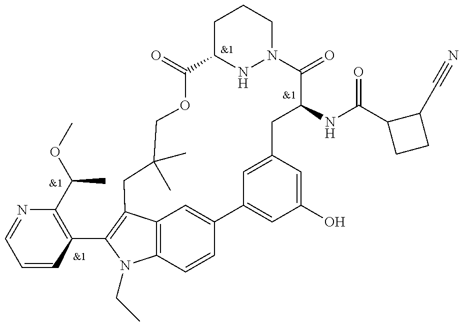 |
| AA226 | 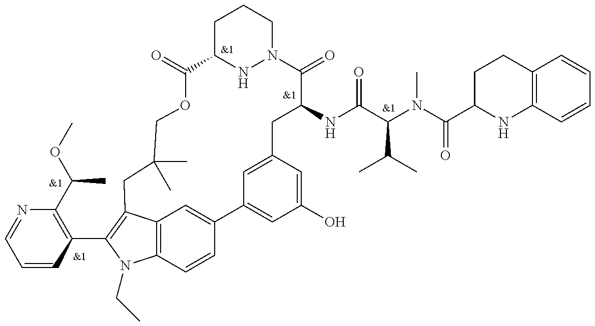 |
| AA227 | 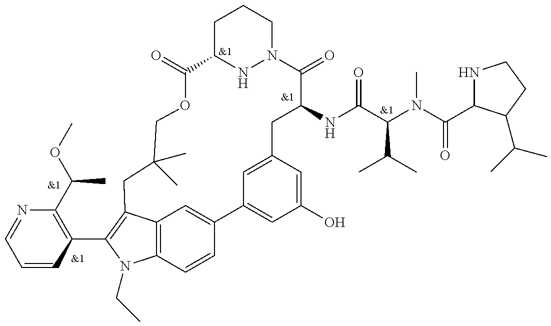 |
| AA228 | 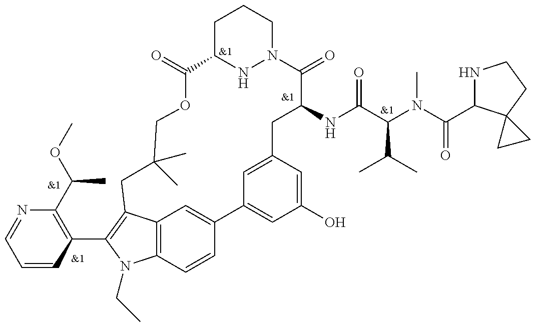 |
| AA229 | 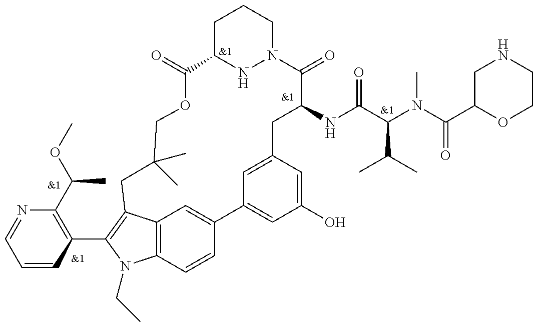 |
| AA230 | 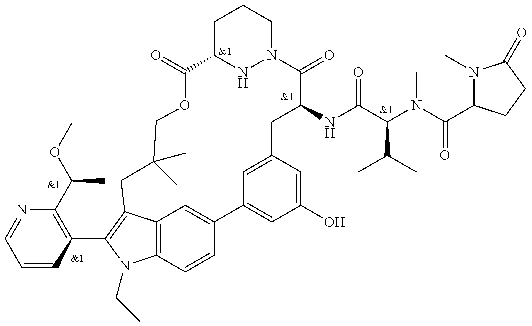 |
| AA231 | 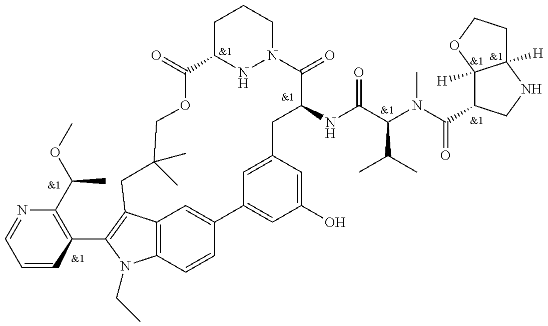 |
| AA232 | 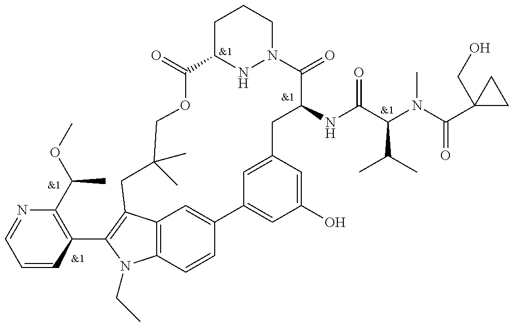 |
| AA233 | 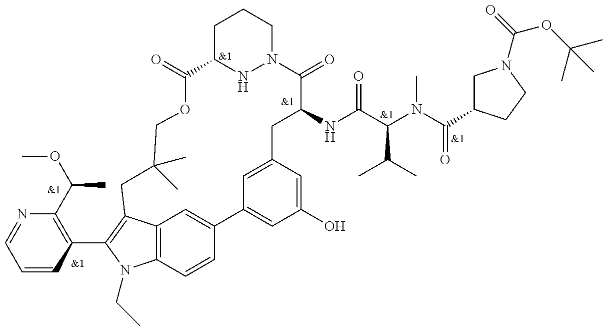 |
| AA234 | 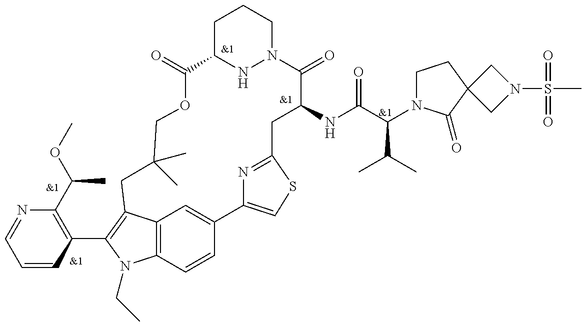 |
| AA235 | 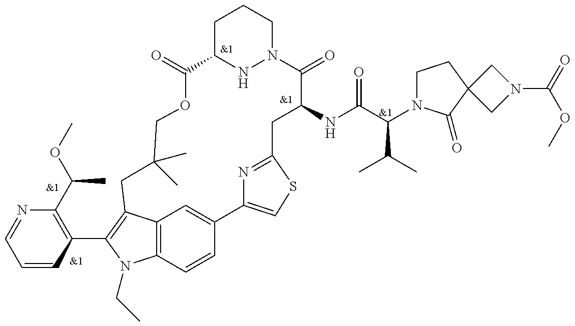 |
| AA236 | 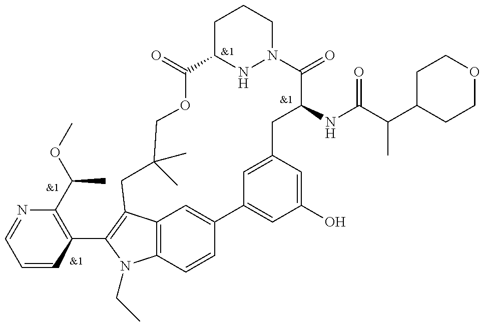 |
| AA237 | 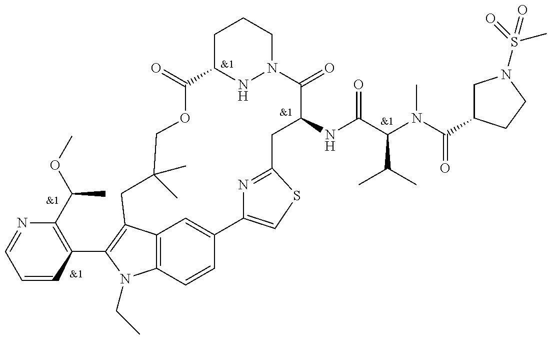 |
| AA238 | 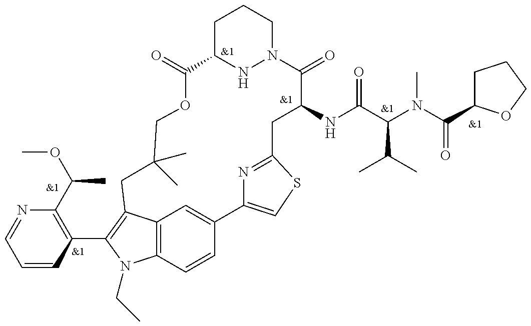 |
| AA239 | 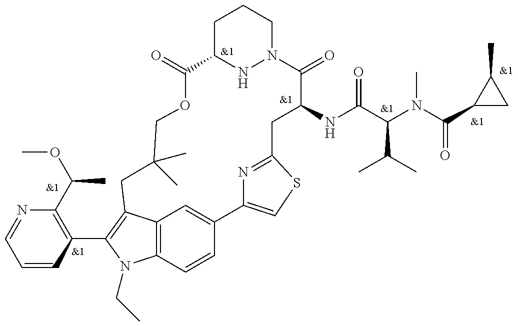 |
| AA240 | 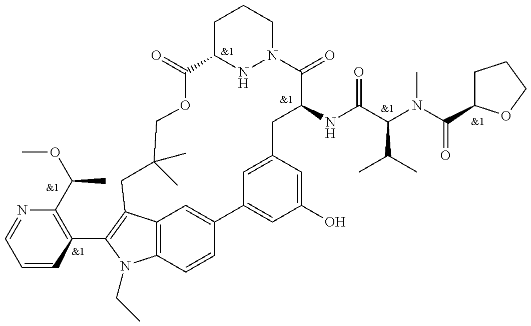 |
| AA241 | 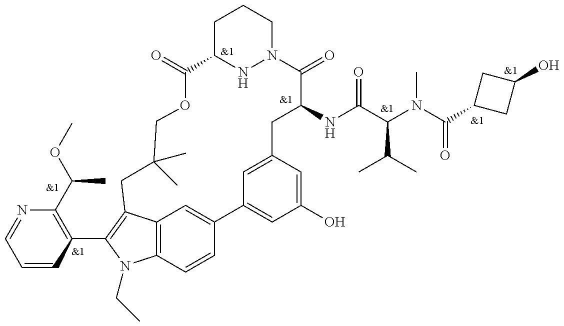 |
| AA242 | 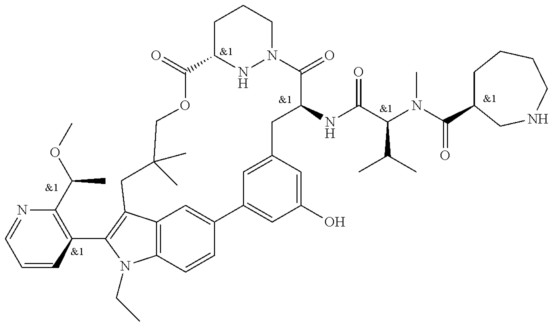 |
| AA243 | 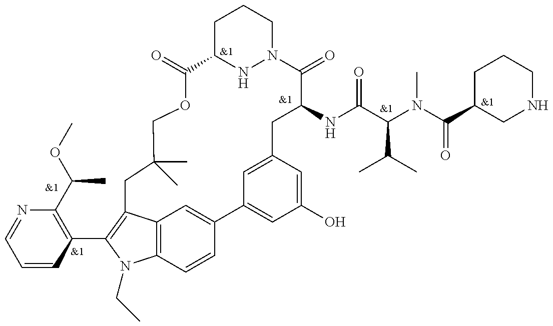 |
| AA244 | 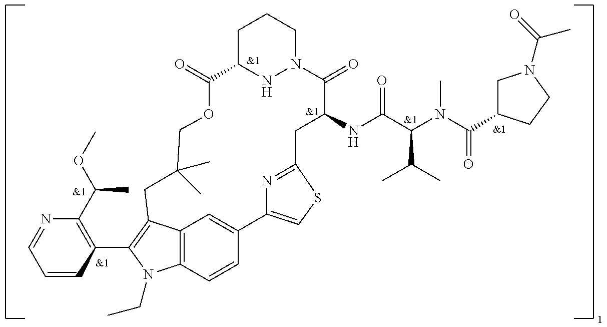 |
| AA245 | 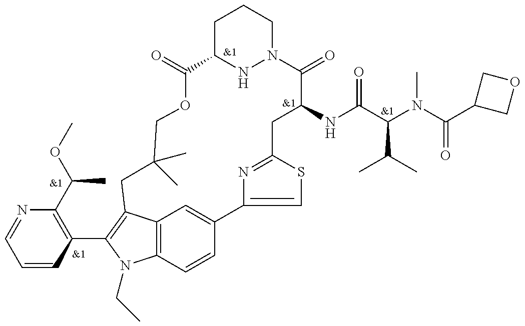 |
| AA246 | 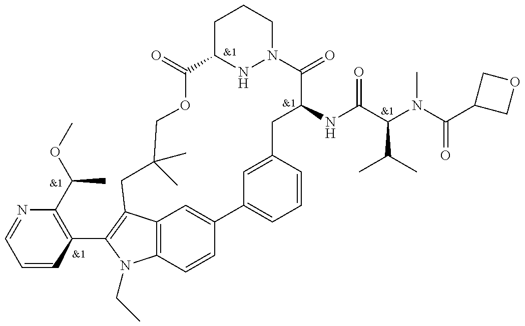 |
| AA247 | 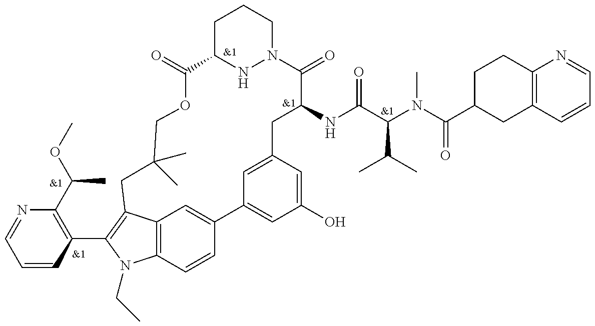 |
| AA248 | 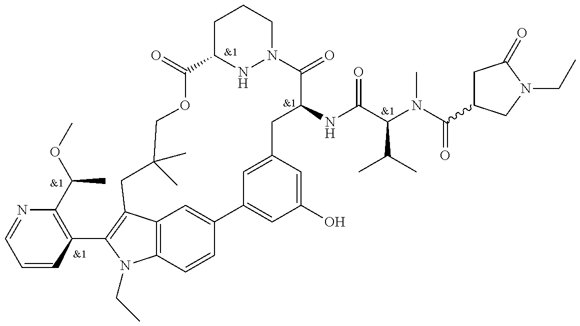 |
| AA249 | 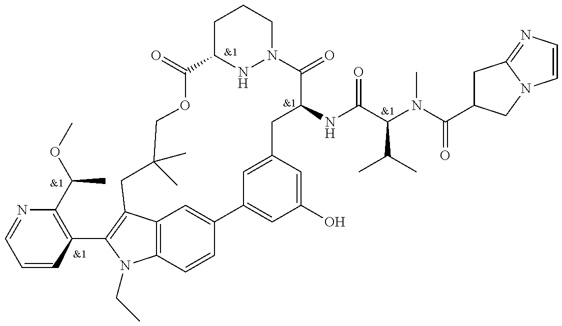 |
| AA250 | 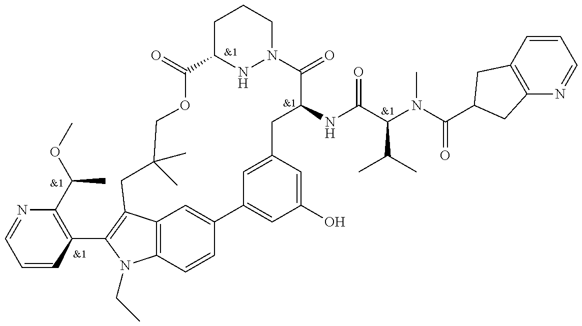 |
| AA251 | 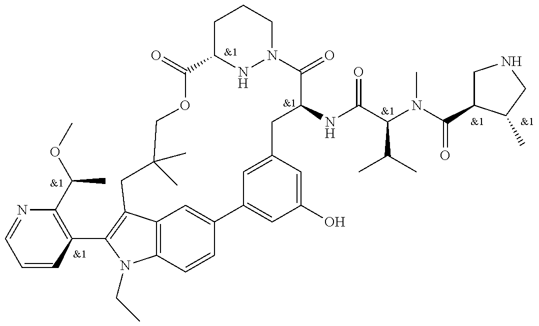 |
| AA252 | 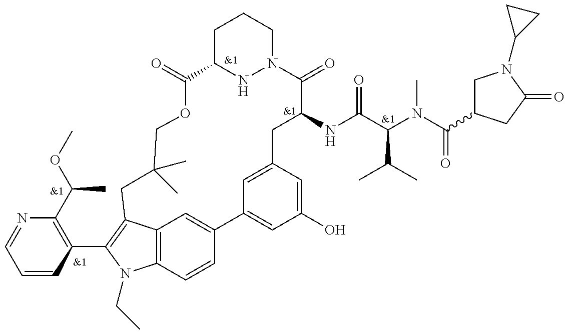 |
| AA253 | 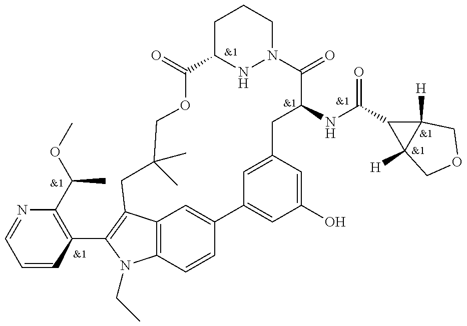 |
| AA254 | 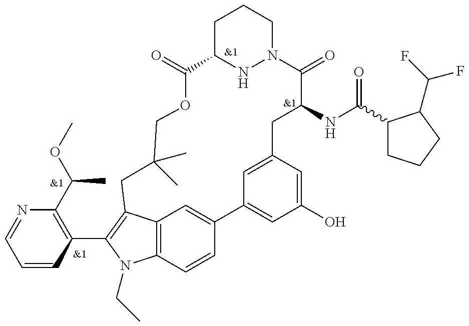 |
| AA255 | 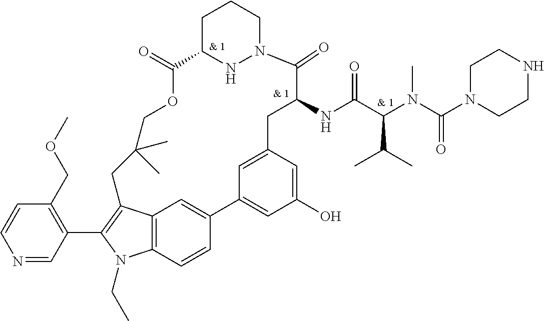 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AA256 | 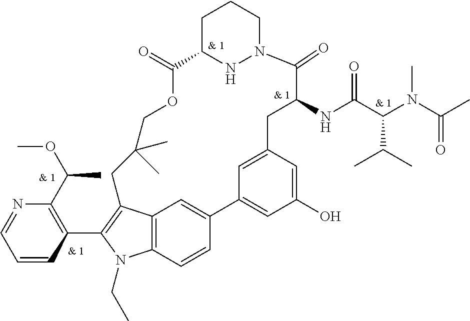 |
| AA257 | 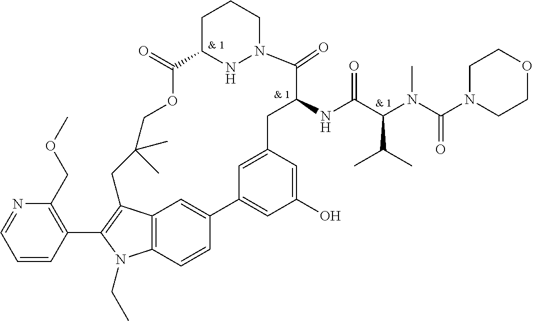 [SMILES: CCn1c(-c2cccnc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AA258 | 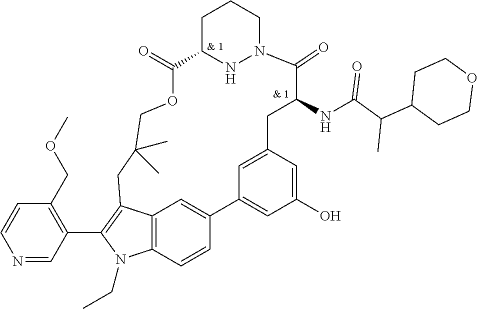 [SMILES: CCn1c(-c2cnccc2COC)c2c3cc(ccc31)-c1cc(O)cc(c1)C[C@H](NC(=O)C(C)C1CCOCC1)C(=O)N1CCC[C@H](N1)C(=O)OCC(C)(C)C2] |
| AA259 | 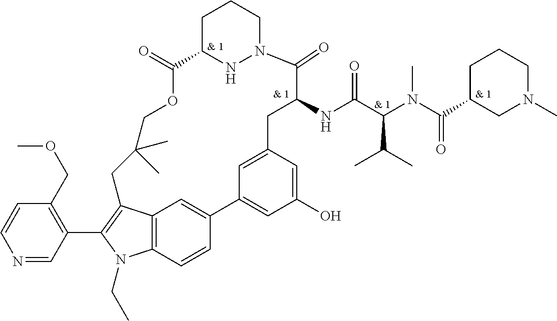 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| AA260 | 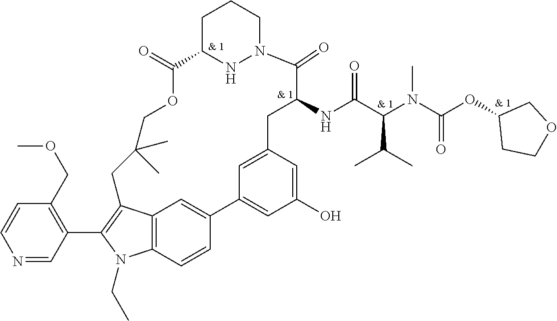 [SMILES: CCn1c(-c2cnccc2COC)c2c3cc(ccc31)-c1cc(O)cc(c1)C[C@H](NC(=O)[C@H](C(C)C)N(C)C(=O)O[C@H]1CCOC1)C(=O)N1CCC[C@H](N1)C(=O)OCC(C)(C)C2] |
| AA261 | 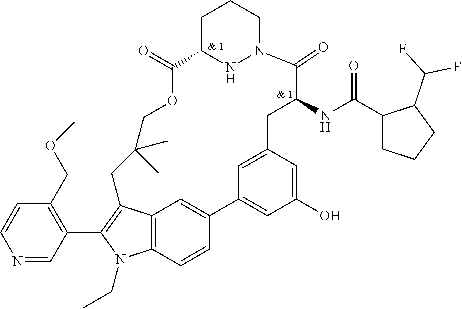 [SMILES: CCn1c(-c2cnccc2COC)c2c3cc(ccc31)-c1cc(O)cc(c1)C[C@H](NC(=O)C1CCCC1C(F)F)C(=O)N1CCC[C@H](N1)C(=O)OCC(C)(C)C2] |
| AA262 | 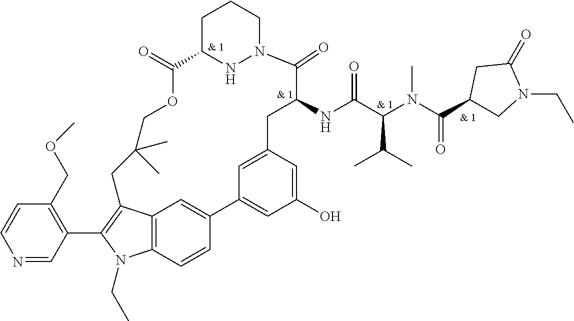 [SMILES: CCN1C[C@@H](C(=O)N(C)[C@H](C(=O)N[C@H]2Cc3cc(O)cc(c3)-c3ccc4c(c3)c(c(-c3cnccc3COC)n4CC)CC(C)(C)COC(=O)[C@@H]3CCCN(N3)C2=O)C(C)C)CC1=O] |
| AA263 | 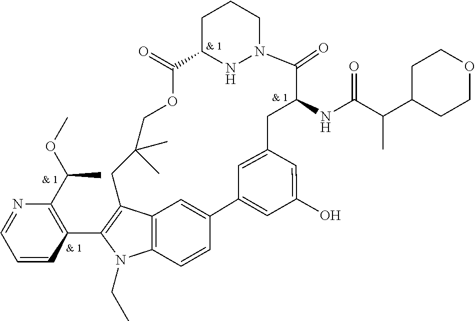 |
| AA264 | 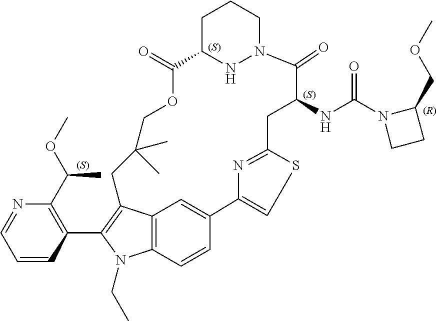 |
| AA265 | 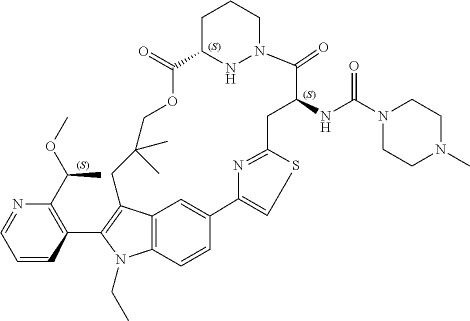 |
| AA266 | 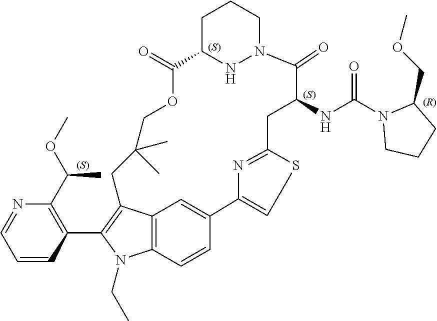 |
| AA267 | 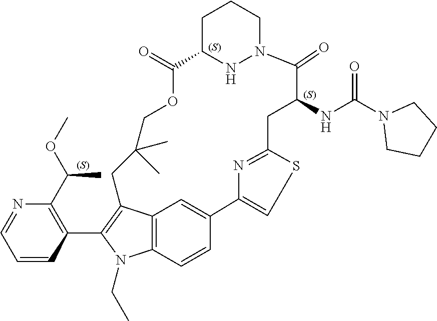 |
| AA268 | 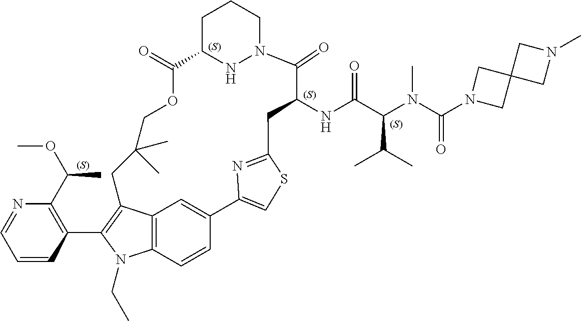 |
| AA270 | 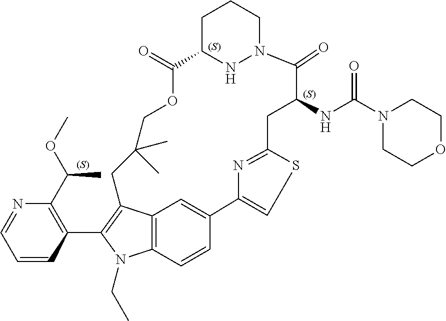 |
| AA271 | 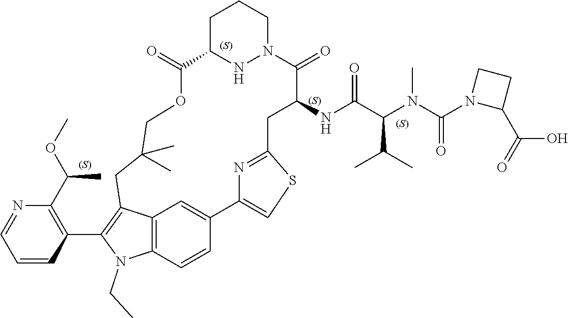 |
| AA272 | 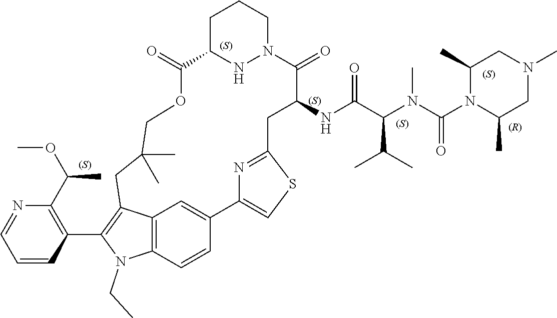 |
| AA273 | 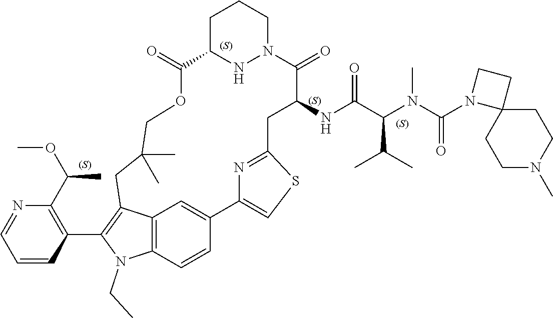 |
| AA274 | 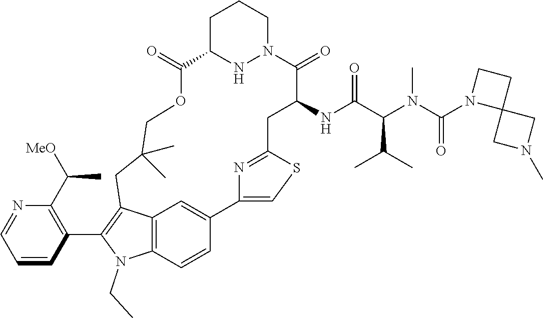 |
| AA275 | 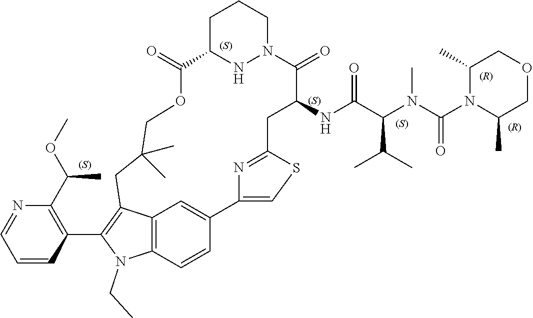 |
| AA276 | 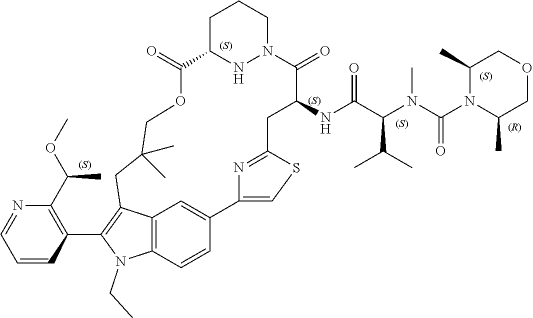 |
| AA277 | 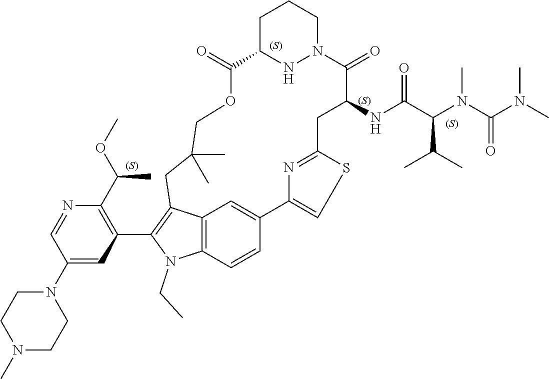 |
| AA278 | 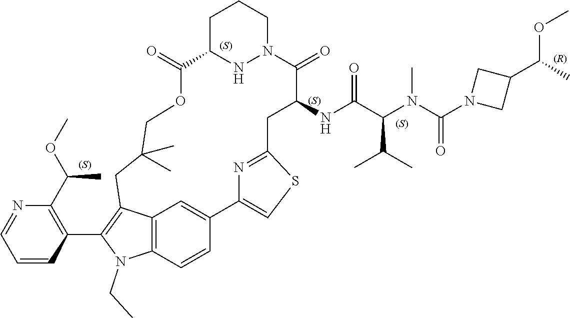 |
| AA279 | 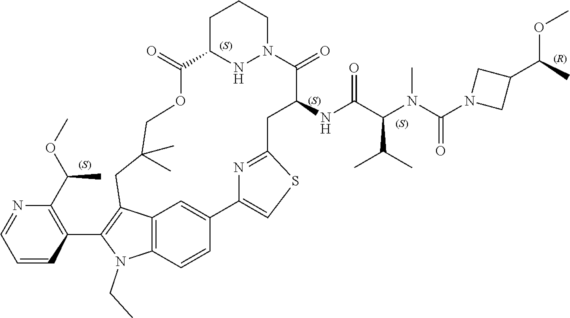 |
| AA280 | 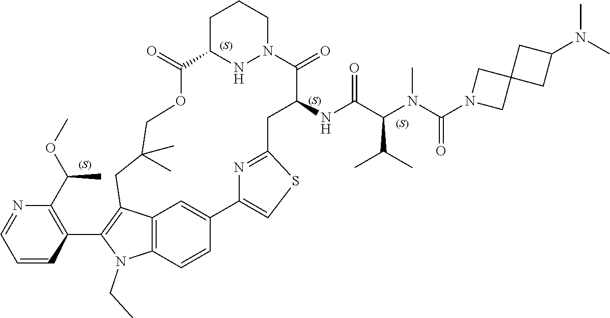 |
| AA281 | 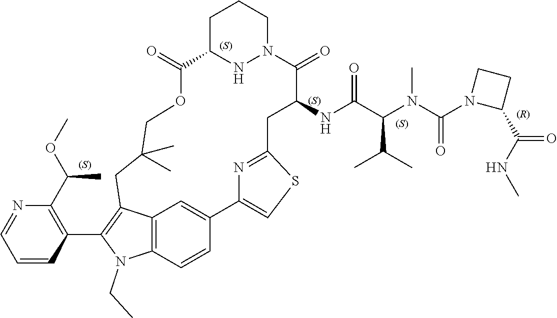 |
| AA282 | 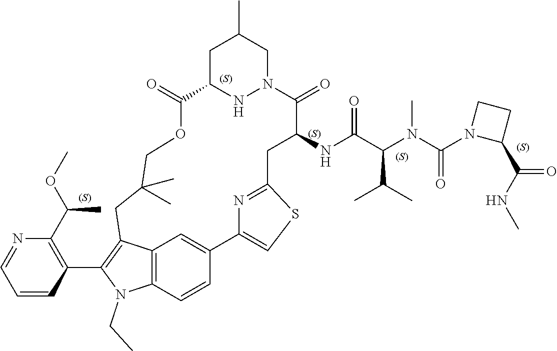 |
| AA283 | 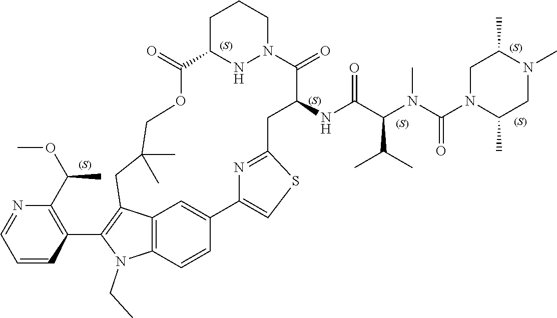 |
| AA284 | 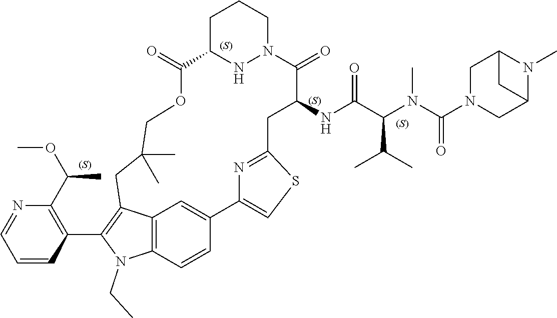 |
| AA285 | 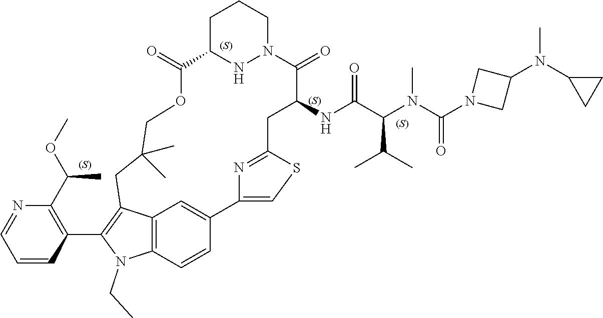 |
| AA286 | 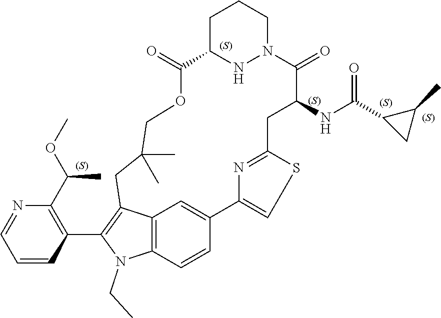 |
| AA287 | 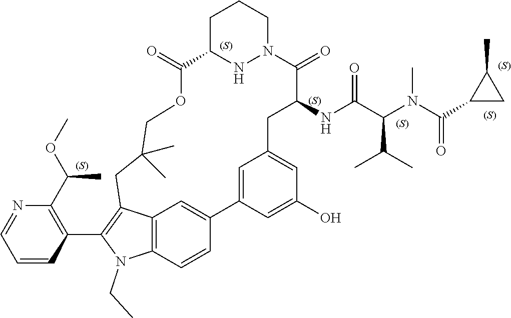 |
| AA288 | 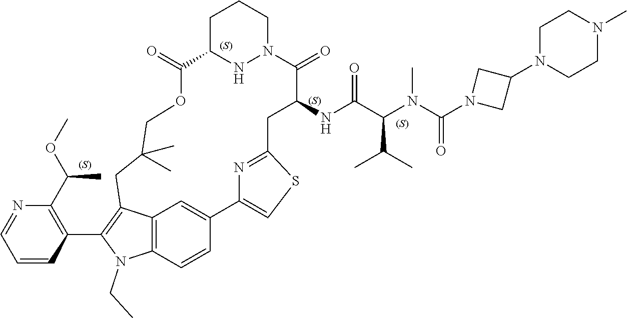 |
| AA289 | 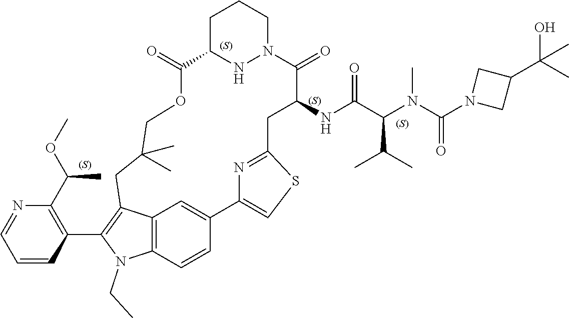 |
| AA290 | 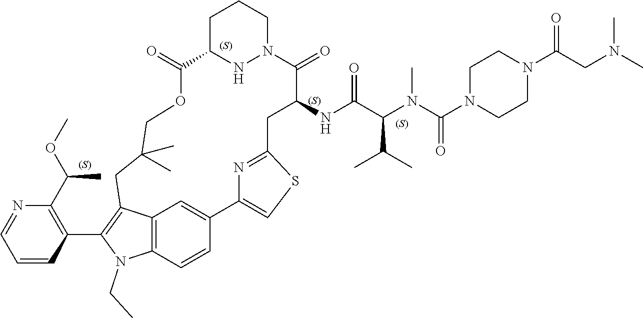 |
| AA291 | 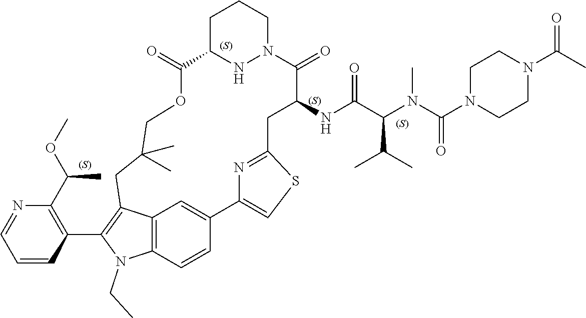 |
| AA292 | 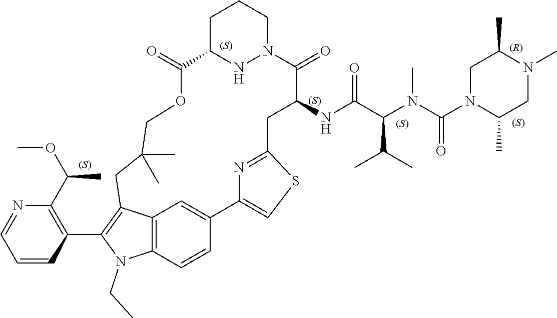 |
| AA293 | 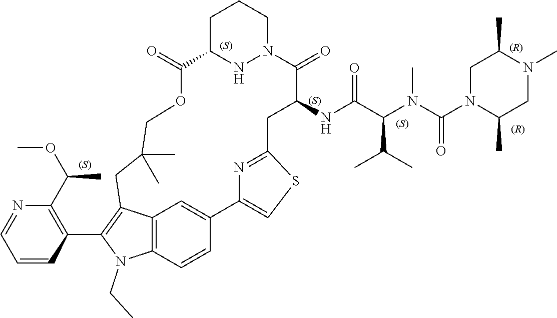 |
| AA294 | 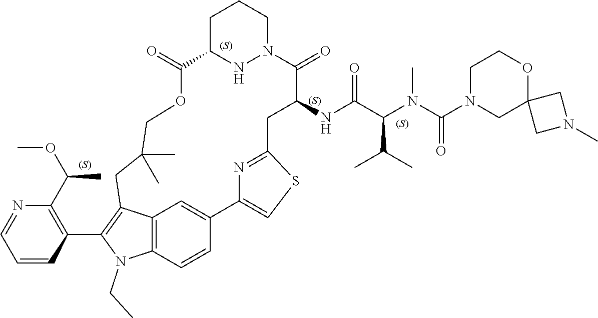 |
| AA295 | 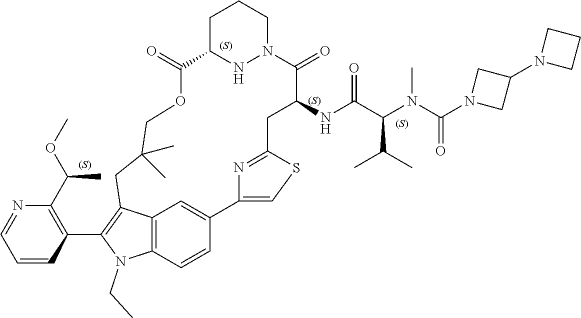 |
| AA296 | 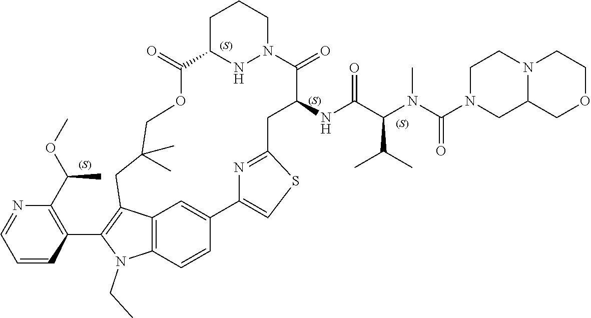 |
| AA297 | 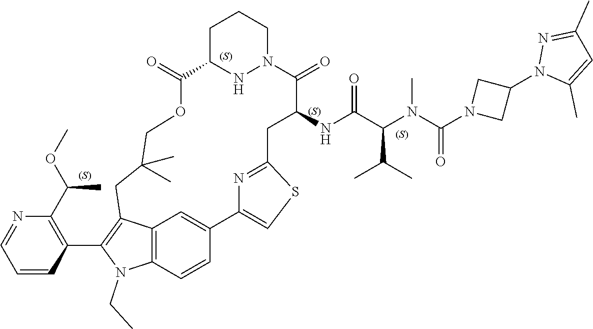 |
| AA298 | 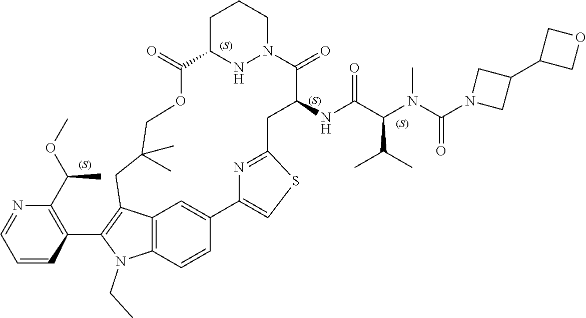 |
| AA299 | 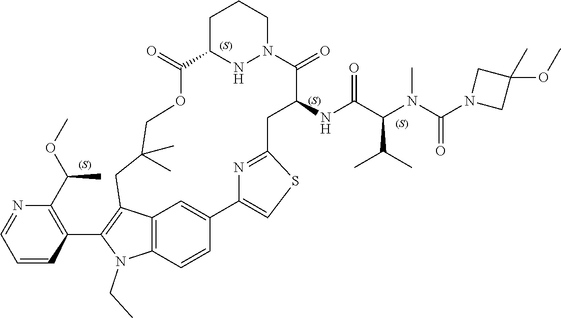 |
| AA300 | 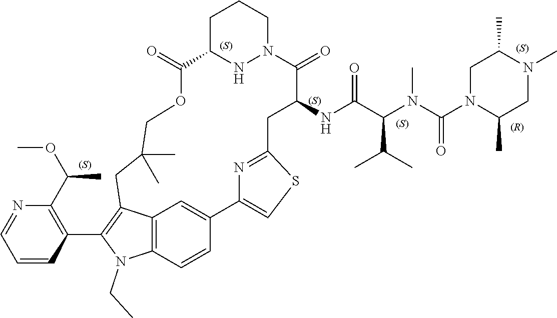 |
| AA301 | 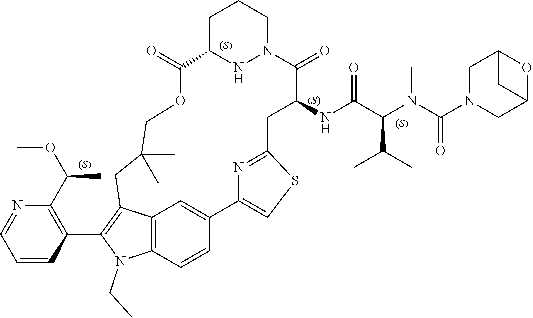 |
| AA302 | 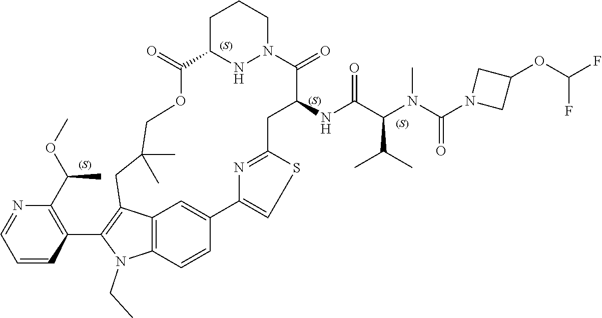 |
| AA303 | 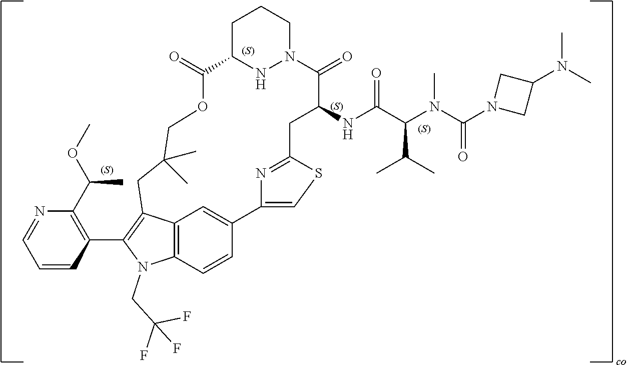 |
| AA304 | 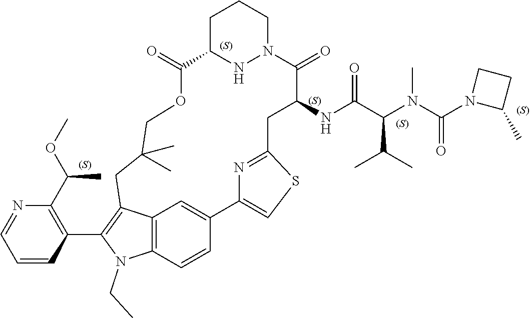 |
| AA305 | 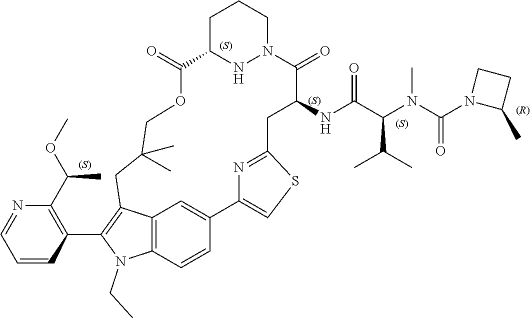 |
| AA306 | 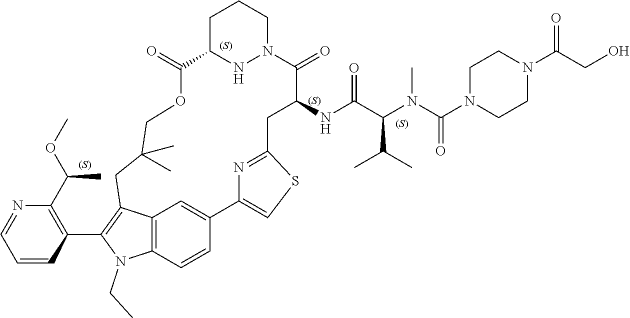 |
| AA307 | 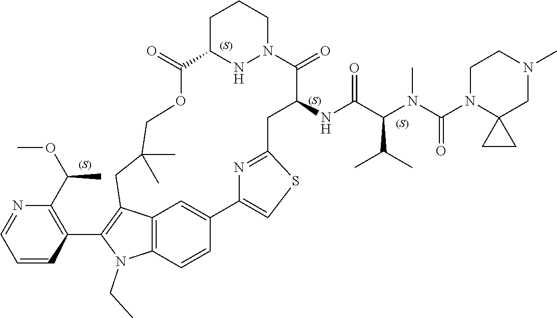 |
| AA308 | 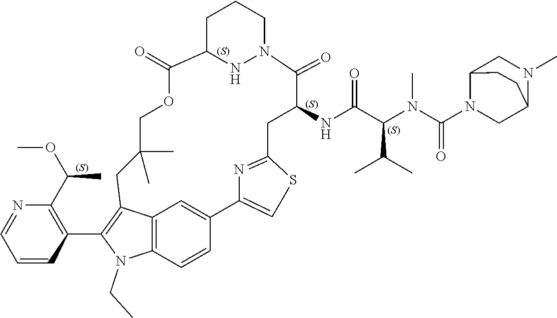 |
| AA309 | 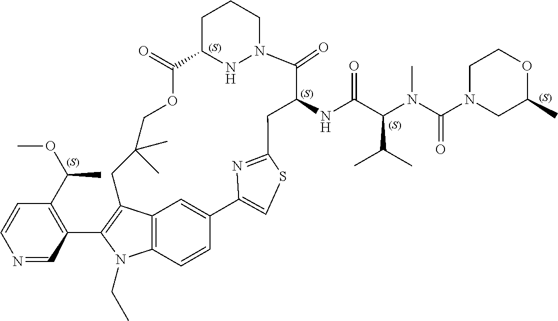 |
| AA310 | 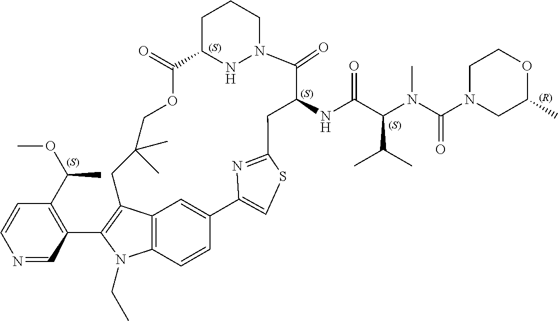 |
| AA311 | 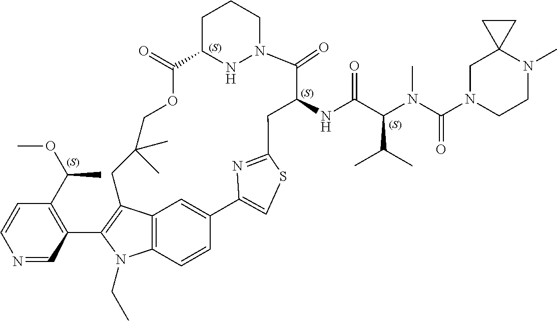 |
| AA312 | 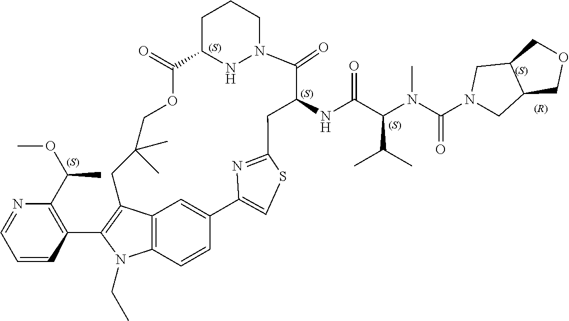 |
| AA313 | 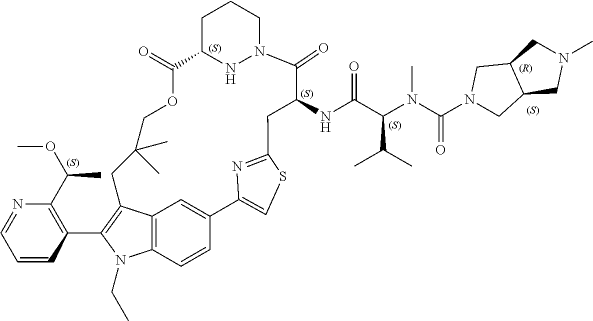 |
| AA314 | 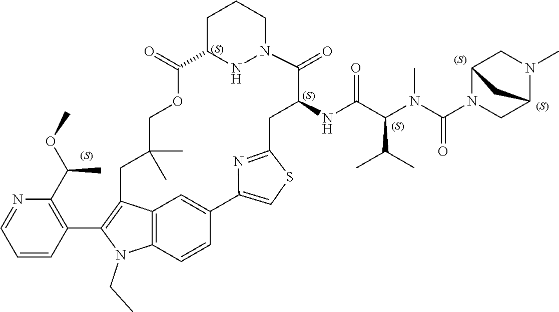 |
| AA315 | 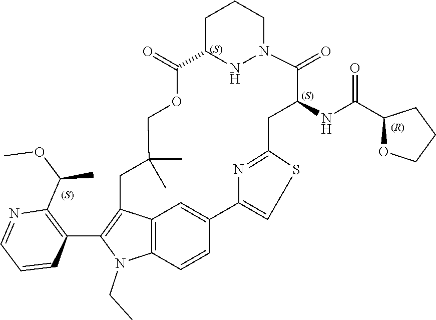 |
| AA316 | 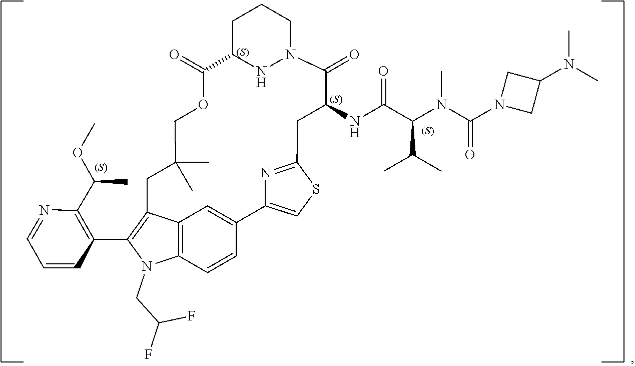 |
| AA317 | 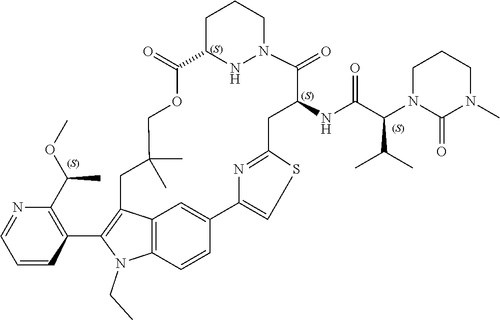 |
| AA318 | 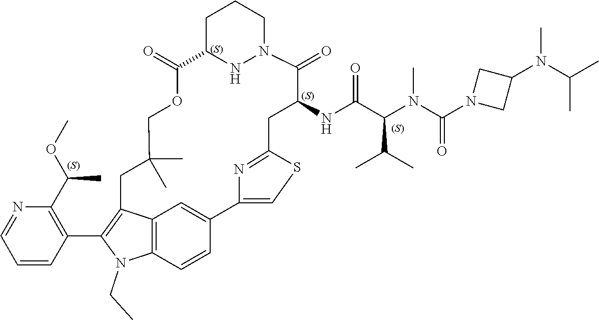 |
| AA319 | 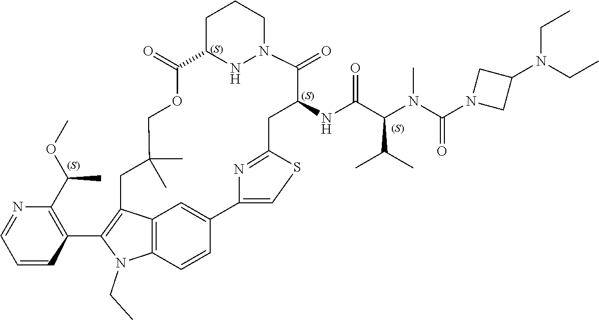 |
| AA320 | 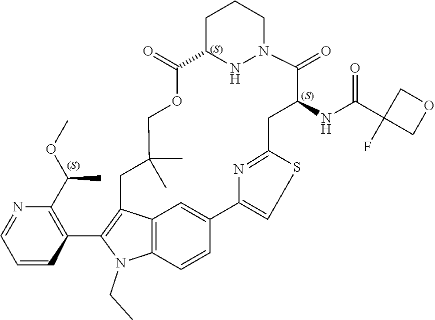 |
| AA321 | 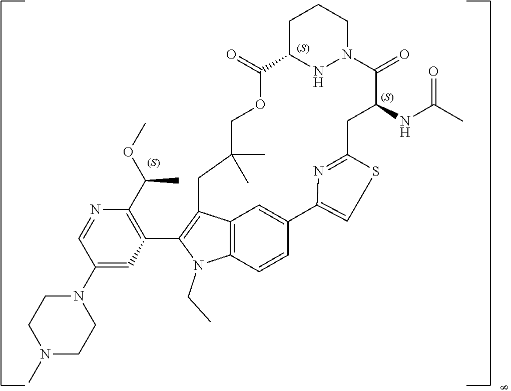 |
| AA322 | 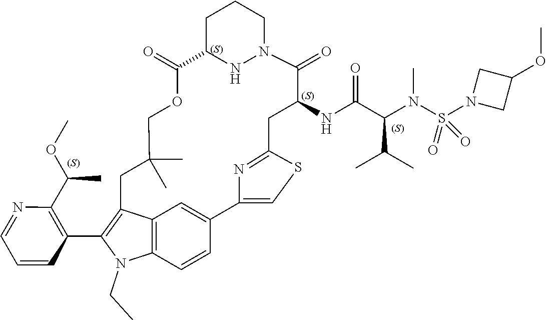 |
| AA323 | 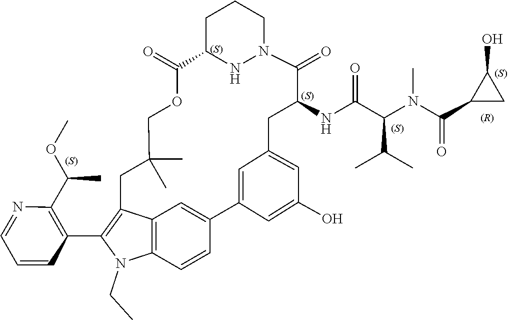 |
| AA324 | 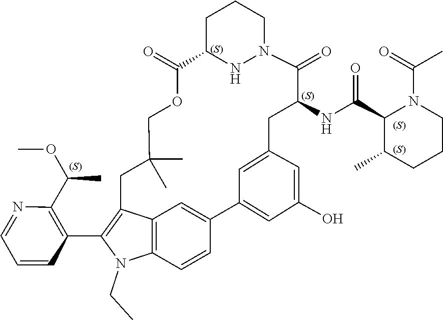 |
| AA325 | 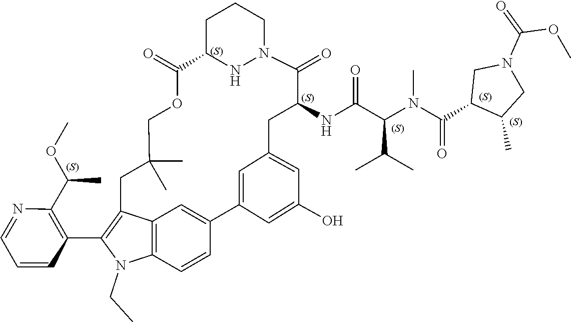 |
| AA326 | 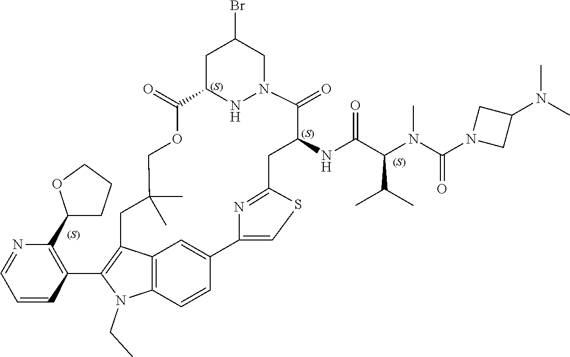 |
| AA327 | 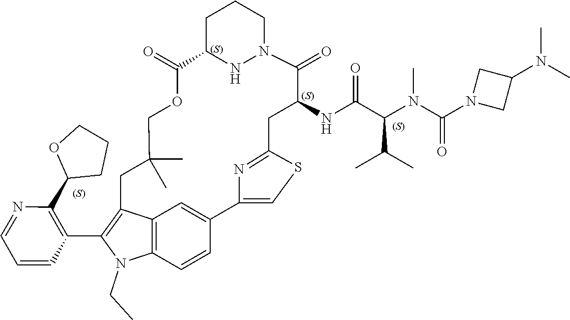 |
| AA328 | 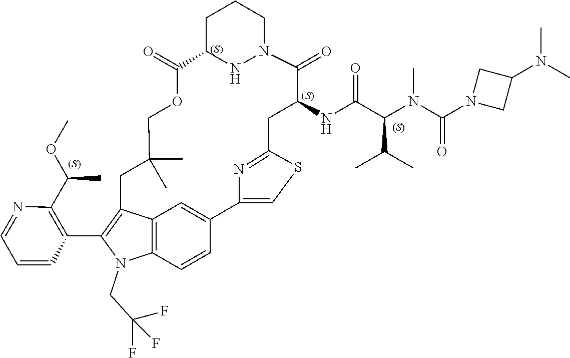 |
| AA329 | 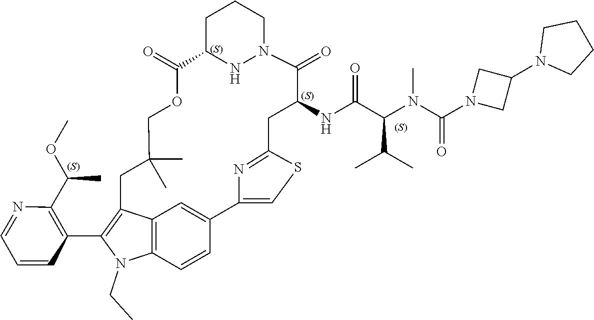 |
| AA330 | 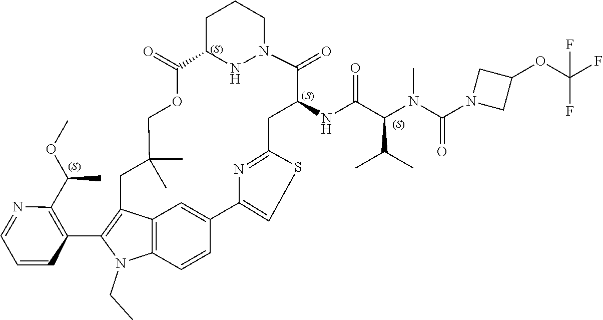 |
| AA331 | 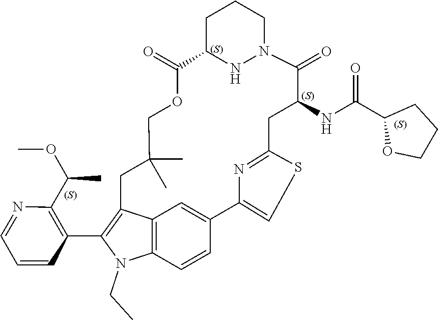 |
| AA332 | 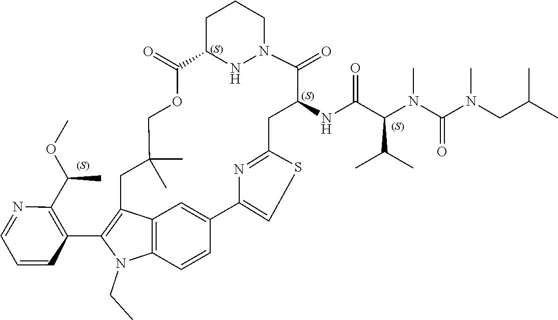 |
| AA333 | 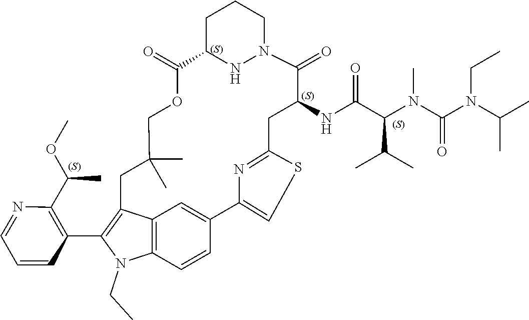 |
| AA334 | 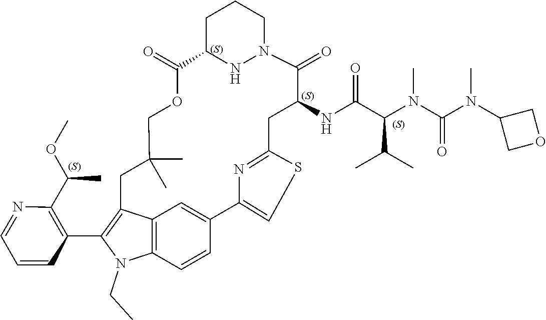 |
| AA335 | 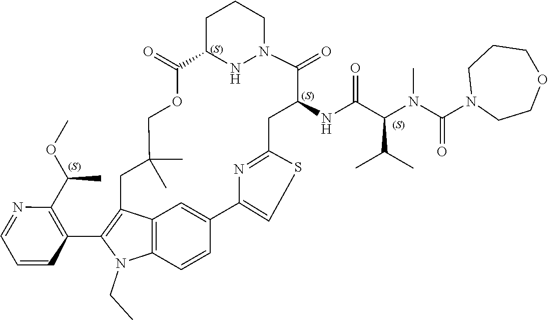 |
| AA336 | 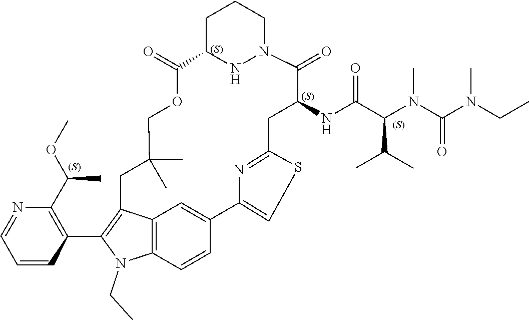 |
| AA337 | 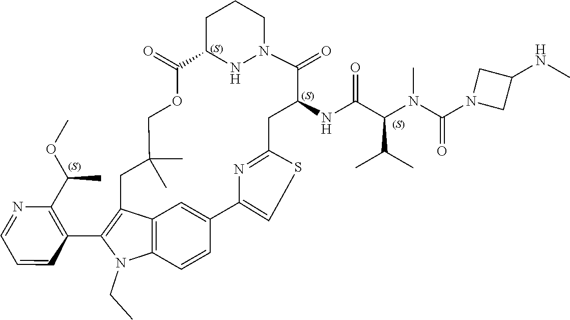 |
| AA338 | 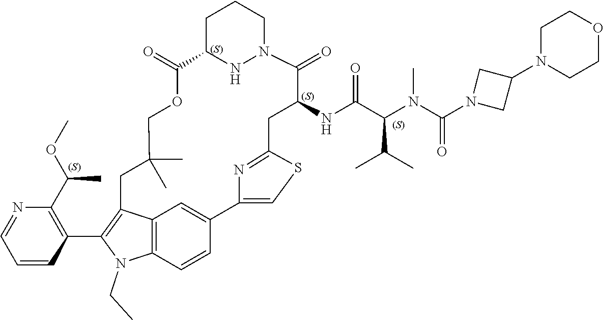 |
| AA339 | 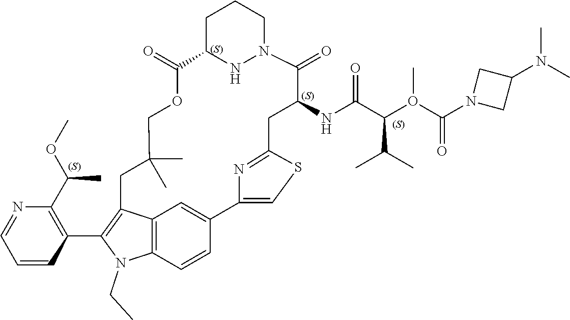 |
| AA340 | 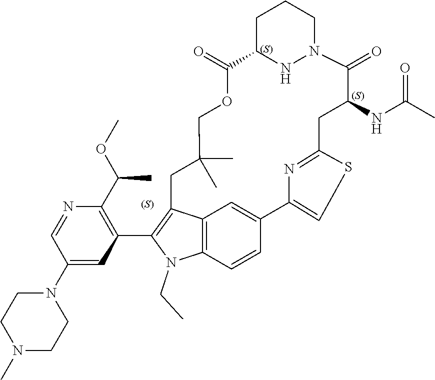 |
| AA341 | 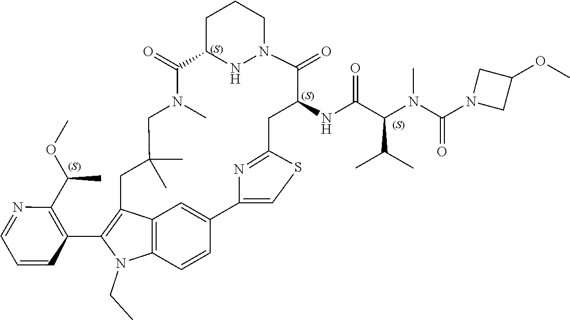 |
| AA342 | 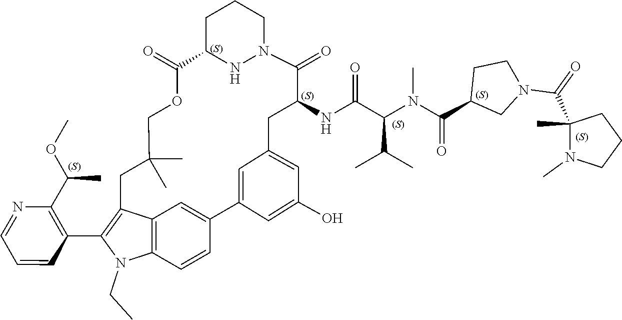 |
| AA343 | 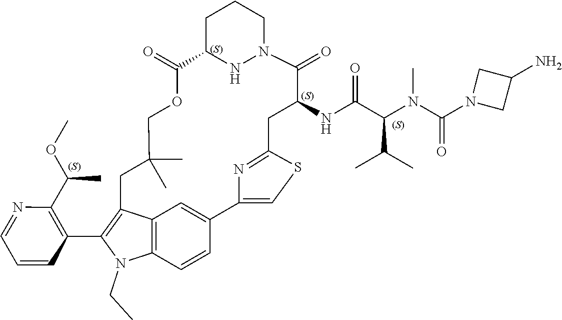 |
| AA344 | 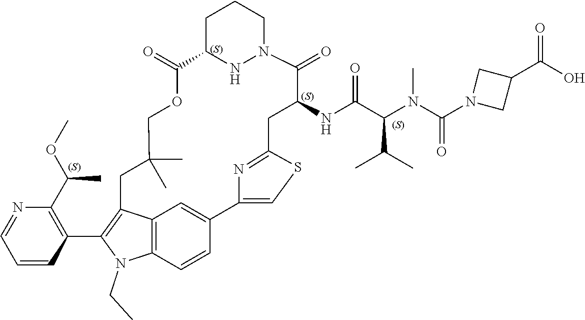 |
| AA345 | 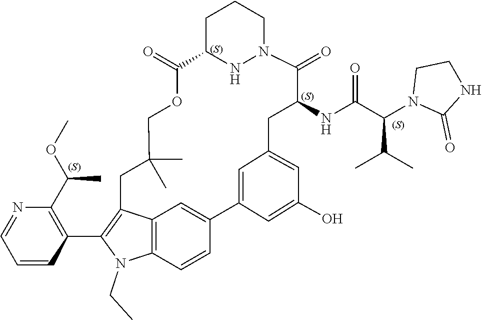 |
| AA346 | 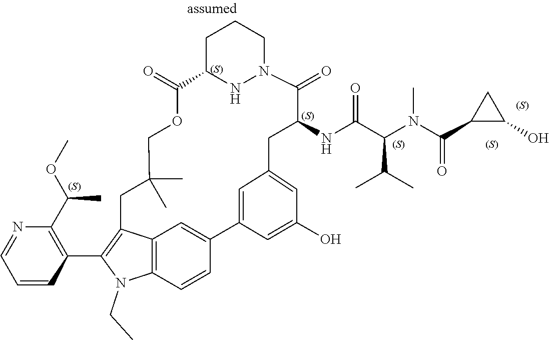 |
| AA347 | 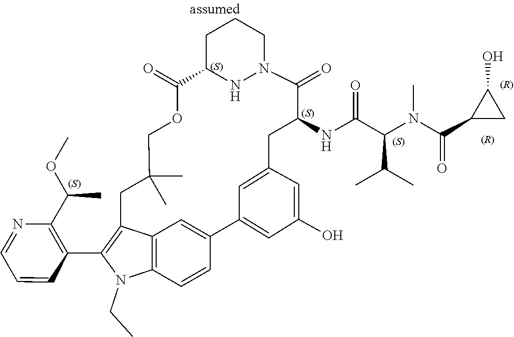 |
| AA348 | 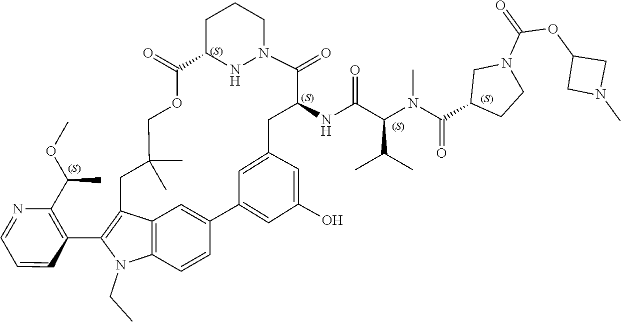 |
| AA349 | 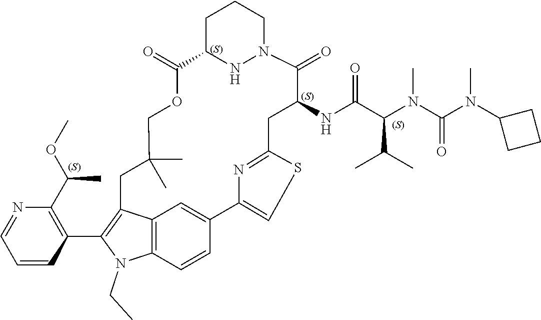 |
| AA350 | 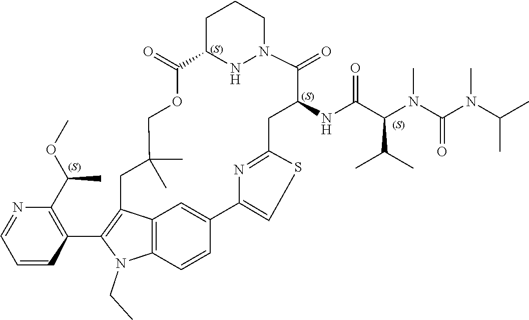 |
| AA351 | 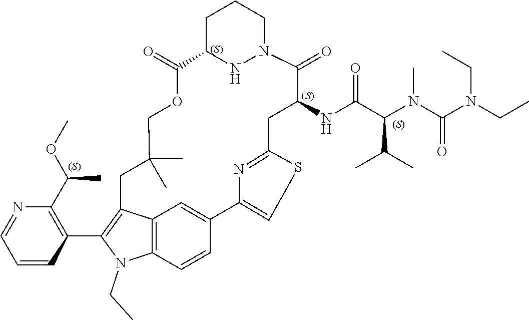 |
| AA352 | 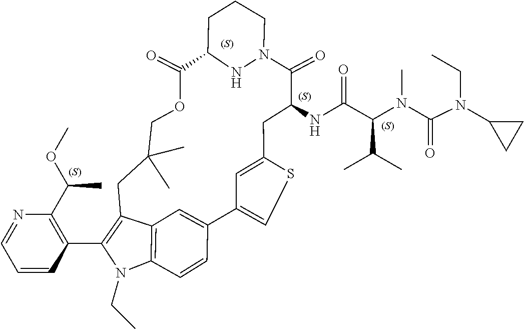 |
| AA353 | 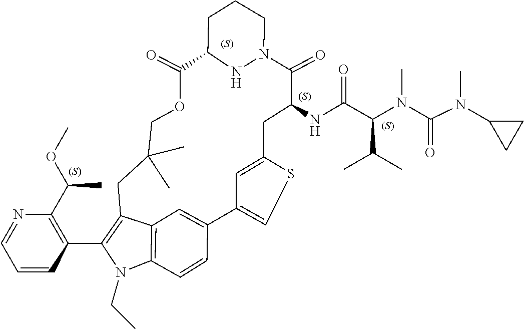 |
| AA354 | 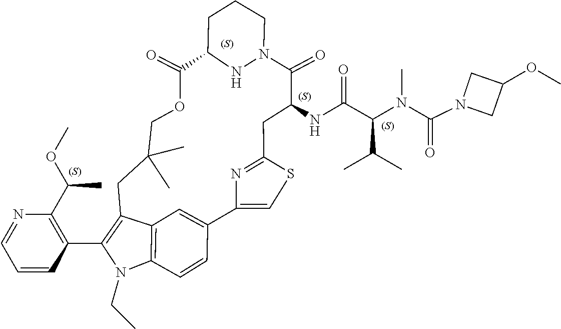 |
| AA355 | 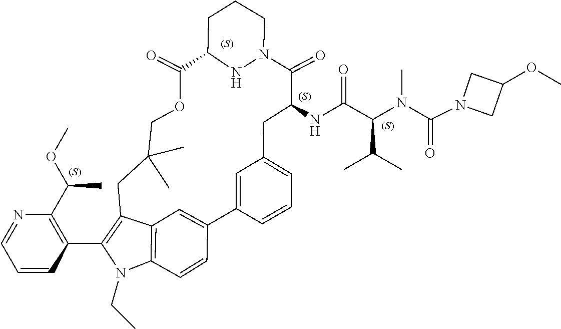 |
| AA356 | 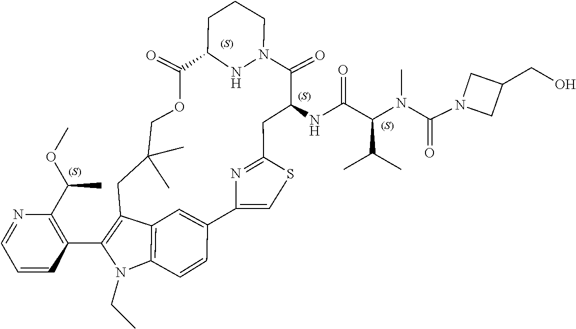 |
| AA357 | 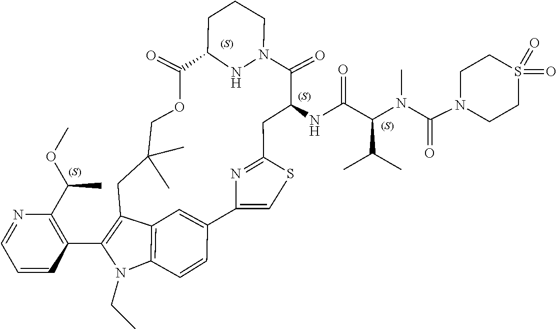 |
| AA358 | 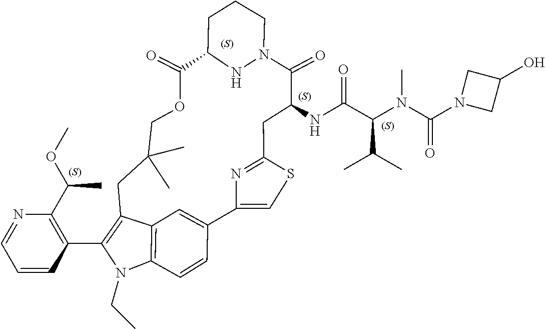 |
| AA359 | 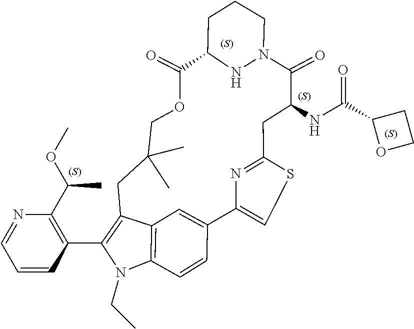 |
| AA360 | 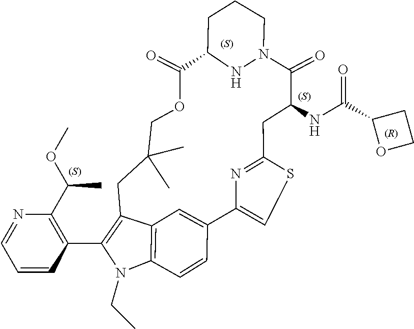 |
| AA361 | 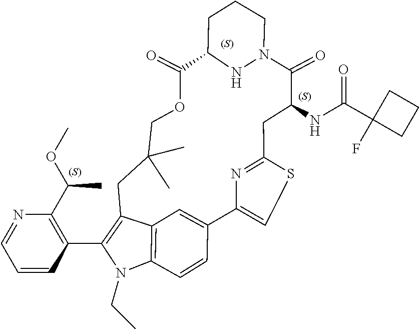 |
| AA362 | 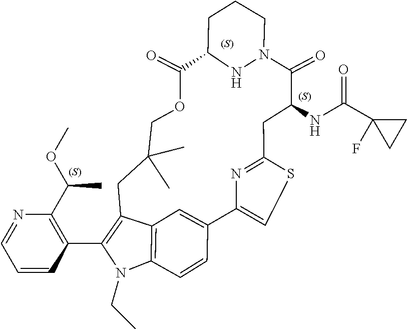 |
| AA363 | 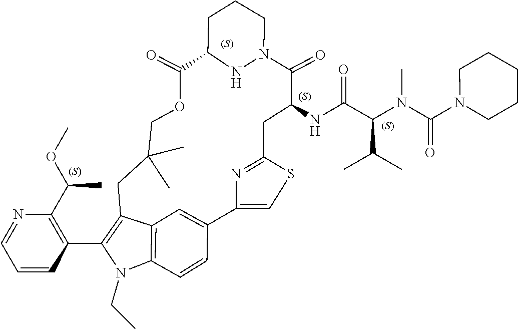 |
| AA364 | 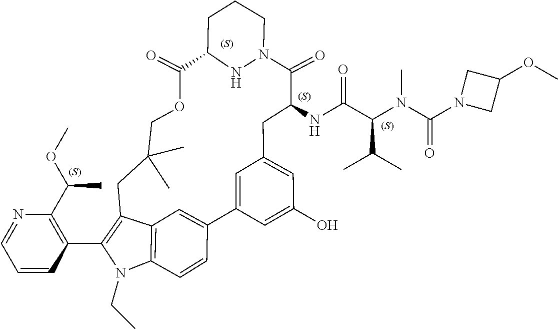 |
| AA365 | 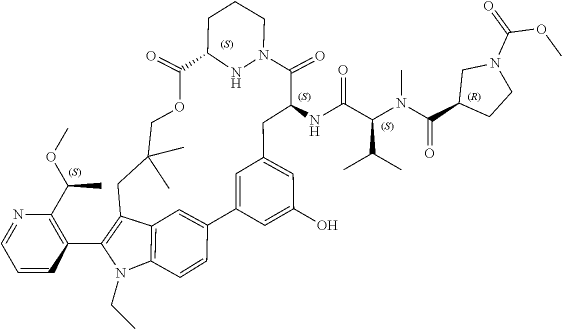 |
| AA366 | 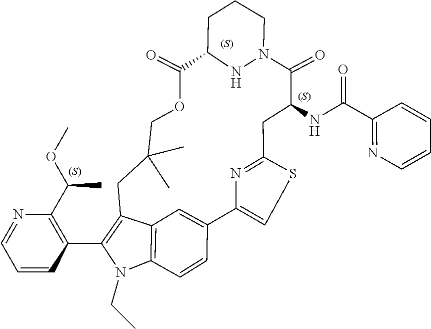 |
| AA367 | 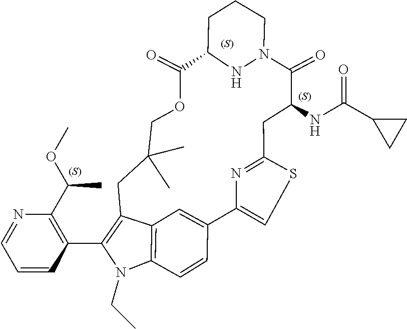 |
| AA368 | 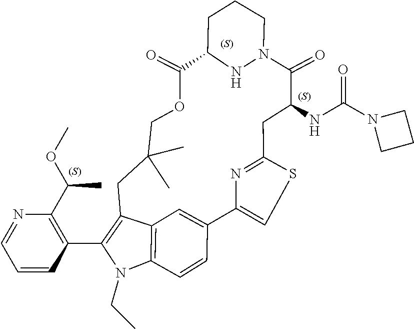 |
| AA369 | 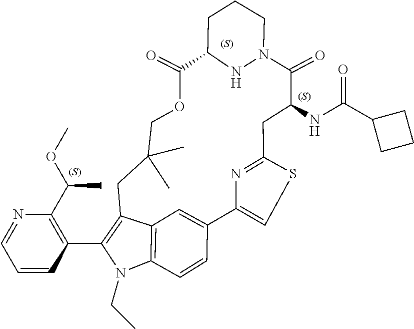 |
| AA370 | 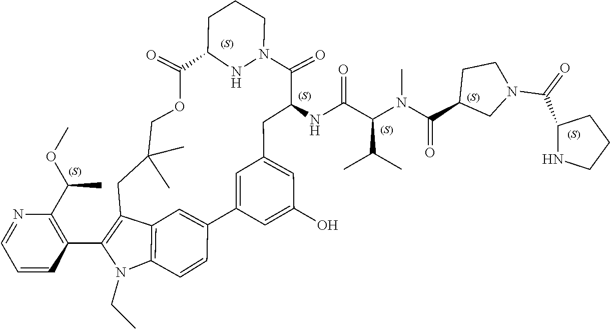 |
| AA371 | 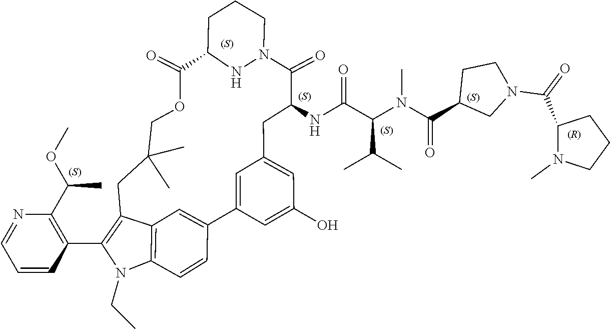 |
| AA372 | 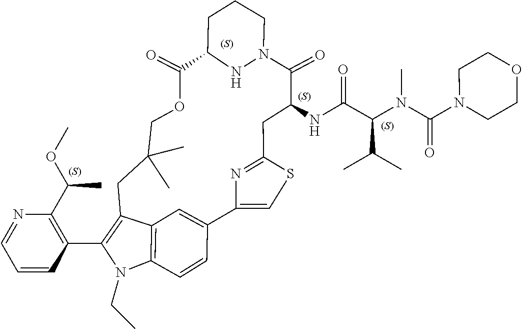 |
| AA373 | 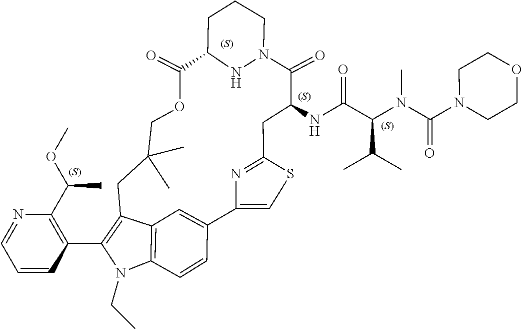 |
| AA374 | 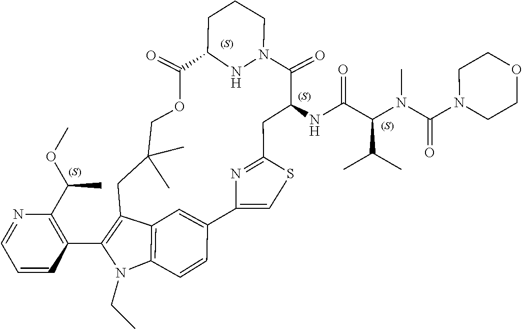 |
| AA375 | 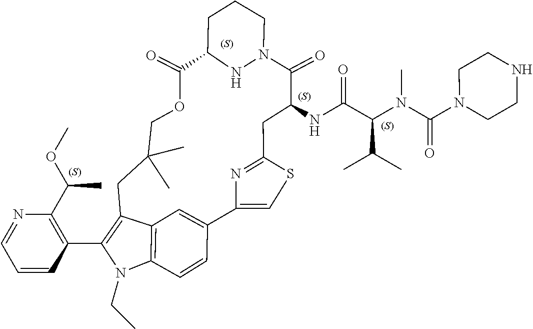 |
| AA376 | 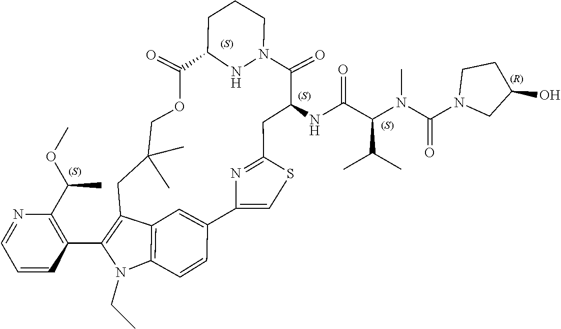 |
| AA377 | 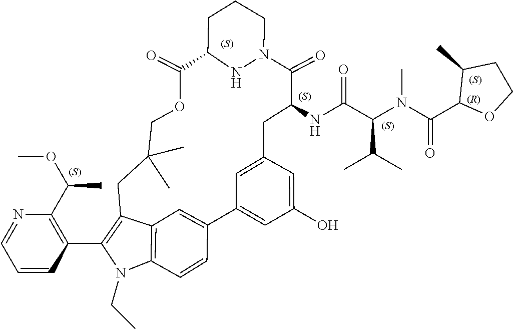 |
| AA378 | 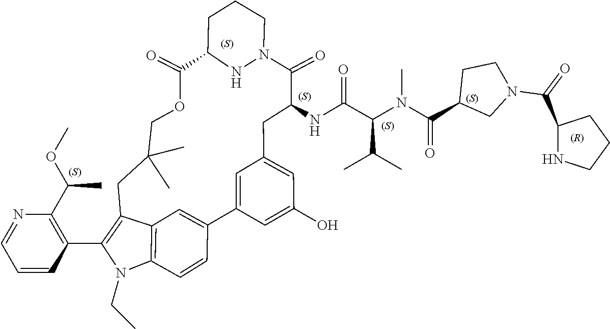 |
| AA379 | 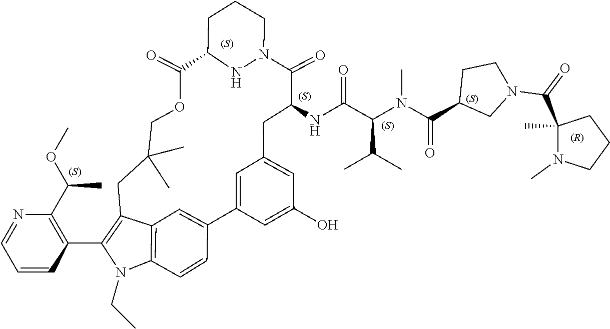 |
| AA380 | 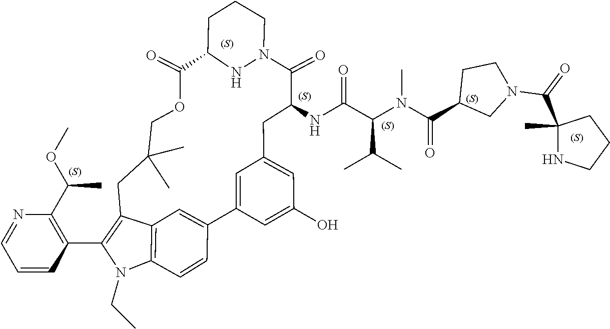 |
| AA381 | 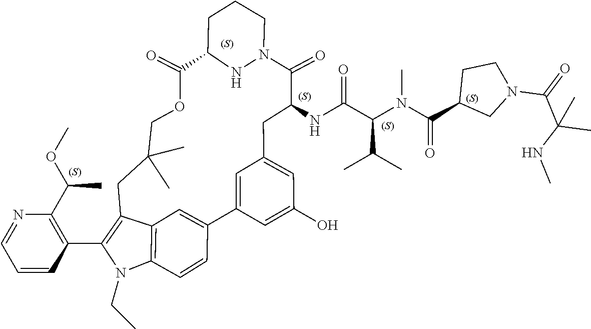 |
| AA382 | 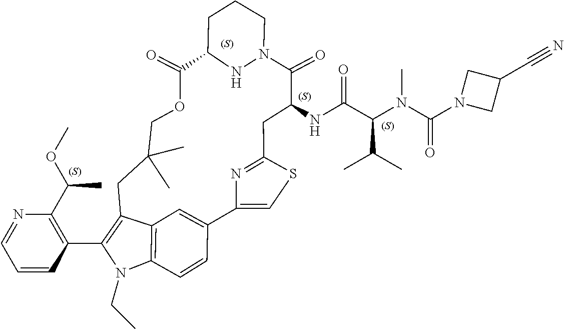 |
| AA383 | 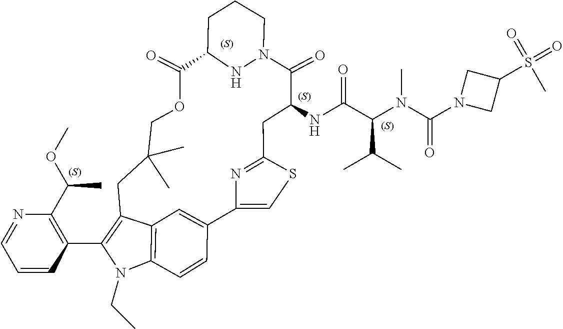 |
| AA384 | 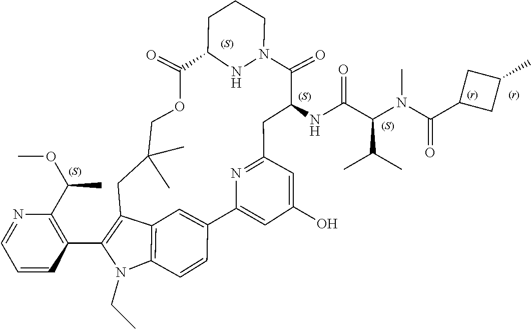 |
| AA385 | 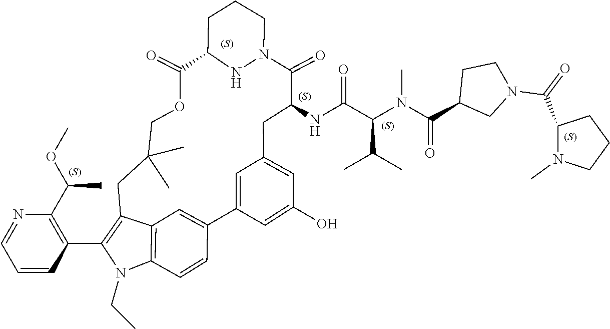 |
| AA386 | 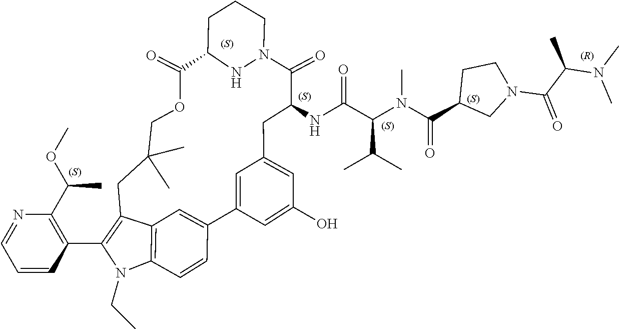 |
| AA387 | 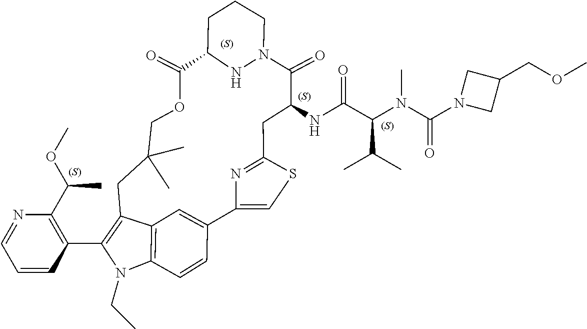 |
| AA388 | 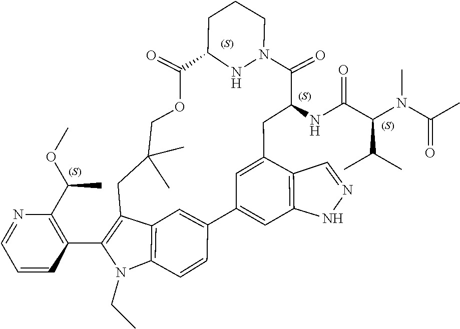 |
| AA389 | 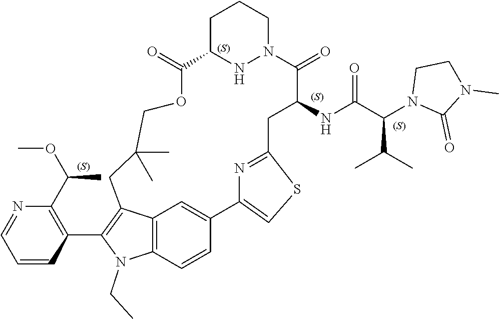 |
| AA391 | 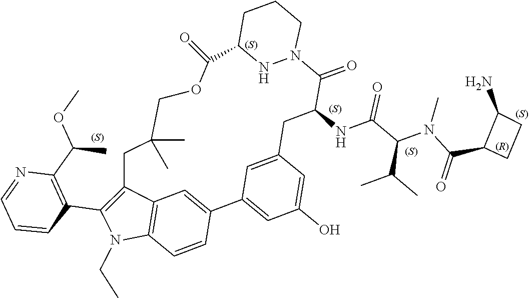 |
| AA392 | 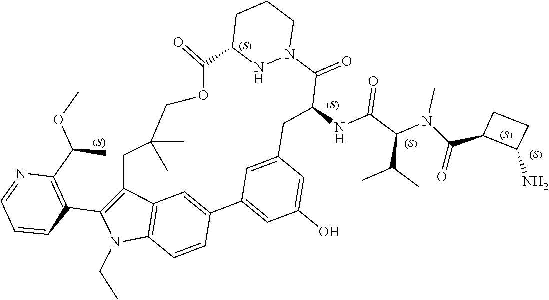 |
| AA393 | 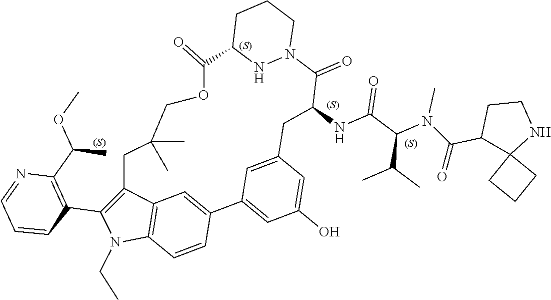 |
| AA394 | 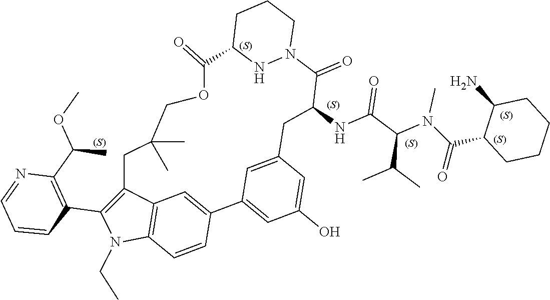 |
| AA395 | 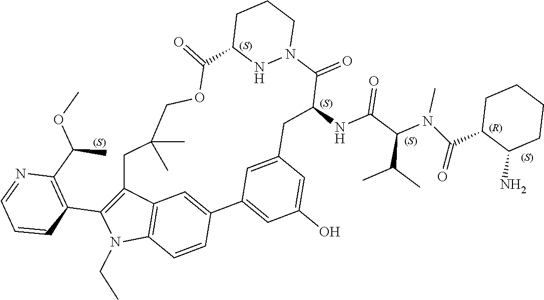 |
| AA396 | 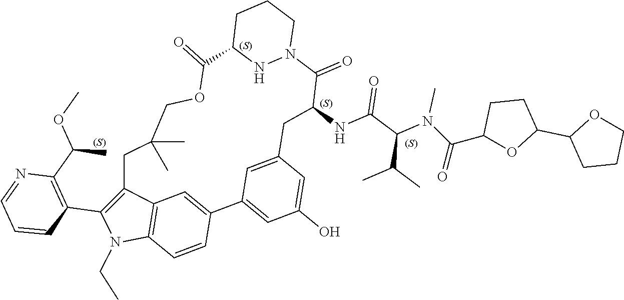 |
| AA397 | 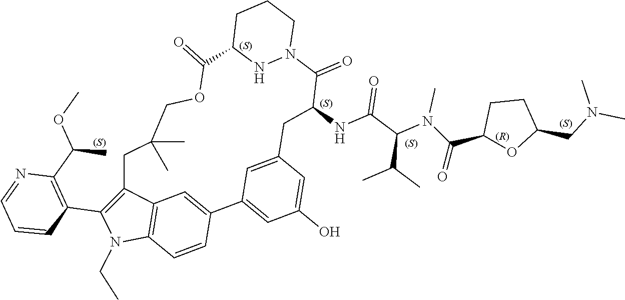 |
| AA398 | 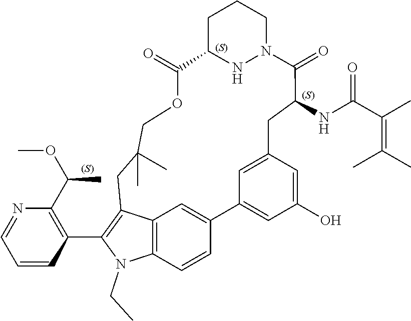 |
| AA399 | 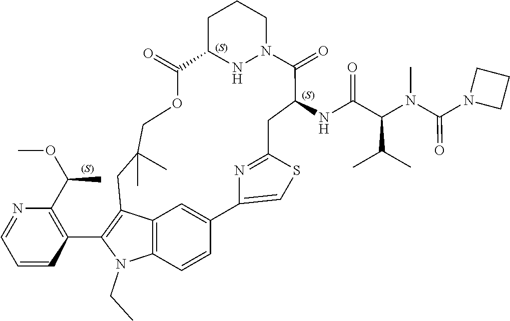 |
| AA400 | 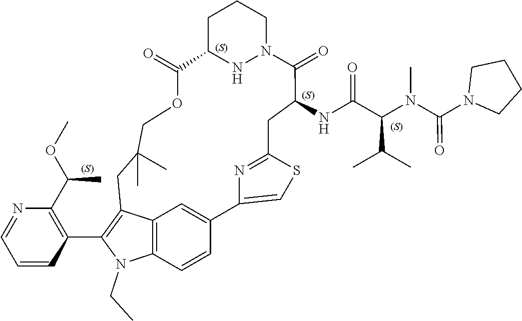 |
| AA401 | 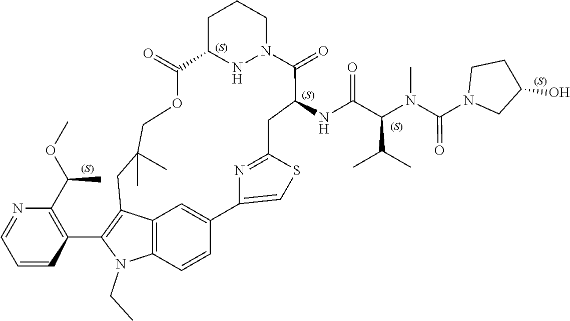 |
| AA402 | 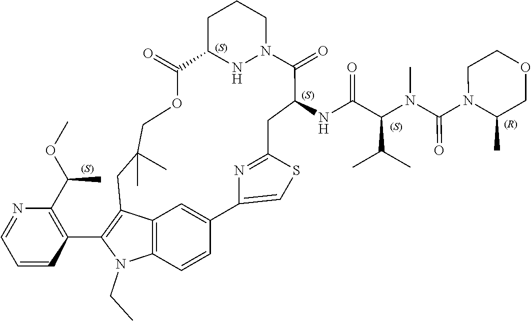 |
| AA403 | 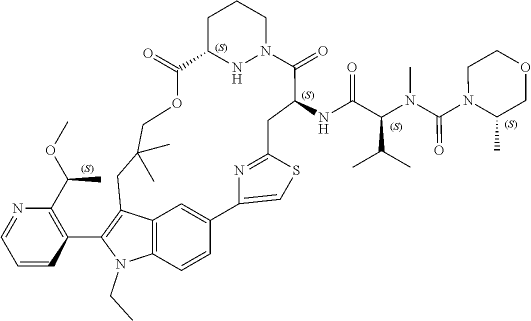 |
| AA404 | 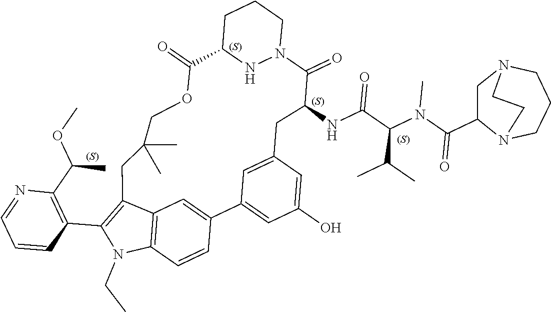 |
| AA405 | 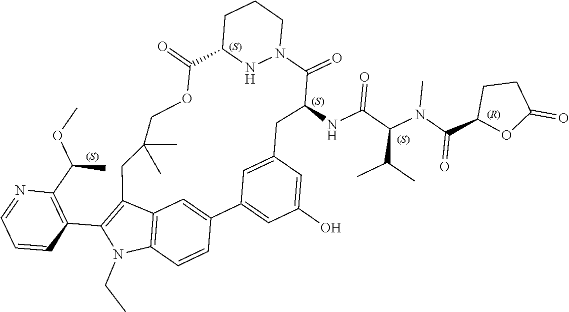 |
| AA406 | 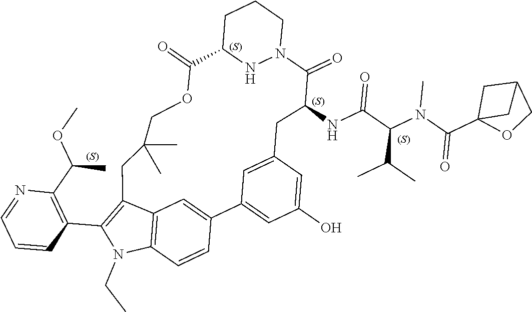 |
| AA407 | 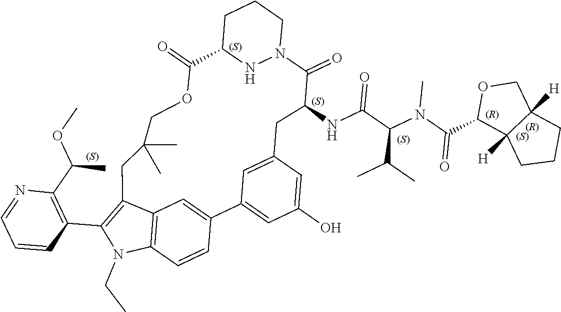 |
| AA408 | 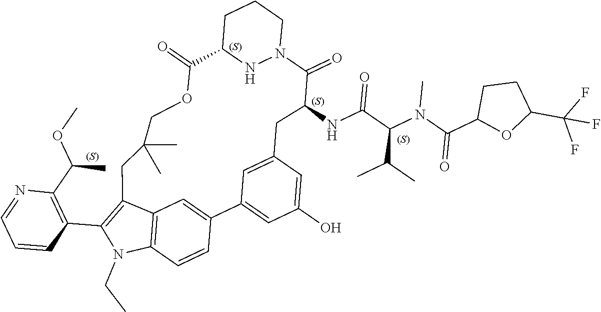 |
| AA409 | 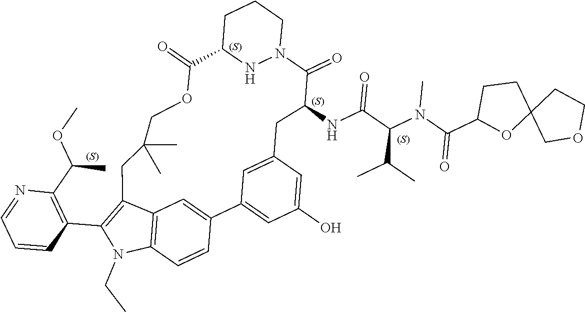 |
| AA410 | 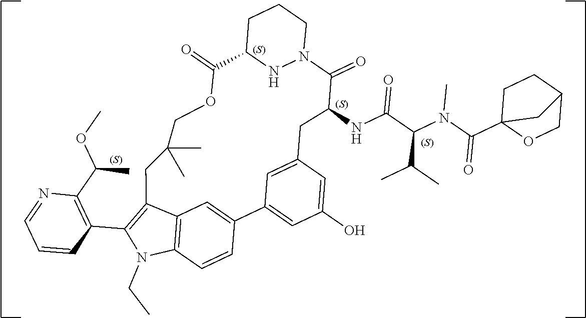 |
| AA411 | 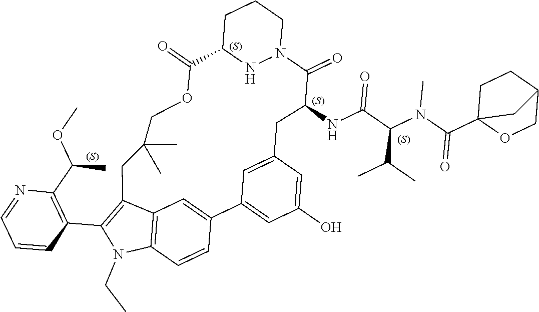 |
| AA412 | 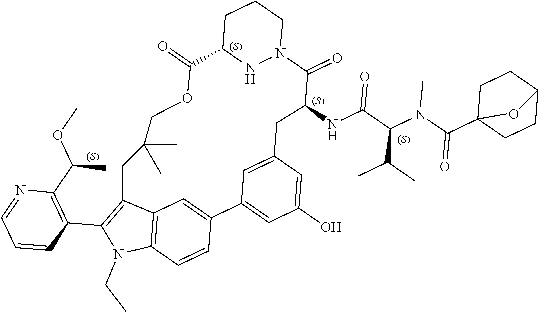 |
| AA413 | 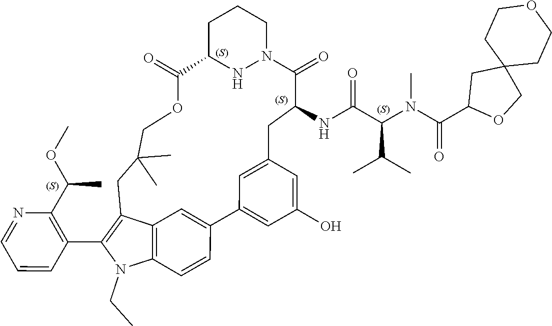 |
| AA414 | 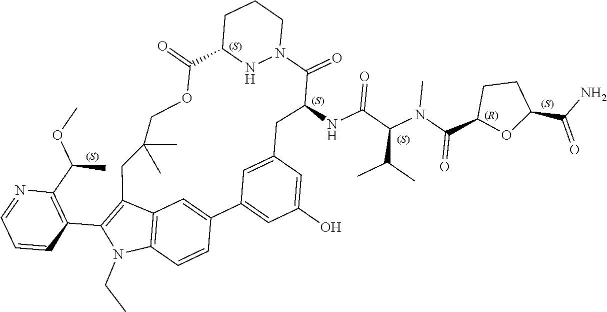 |
| AA415 | 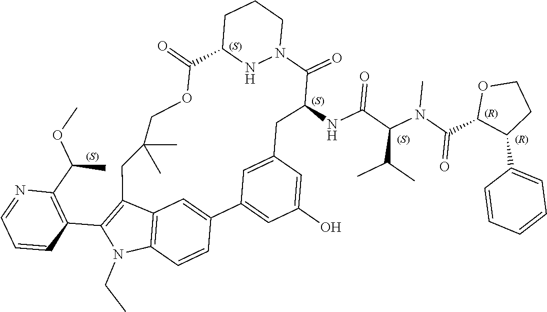 |
| AA416 | 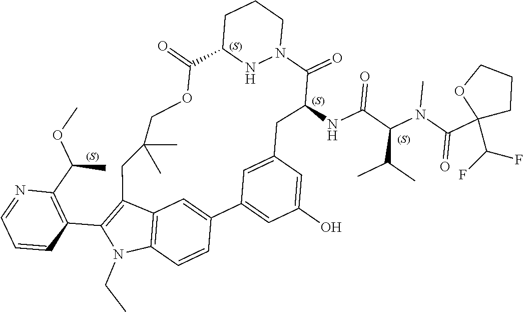 |
| AA417 | 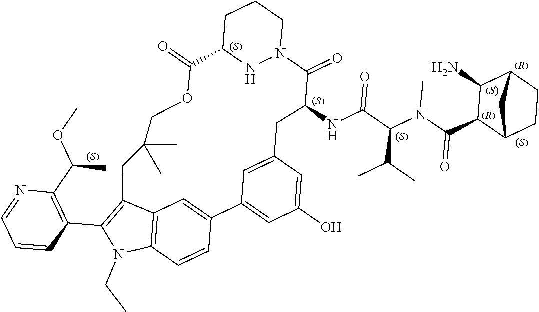 |
| AA418 | 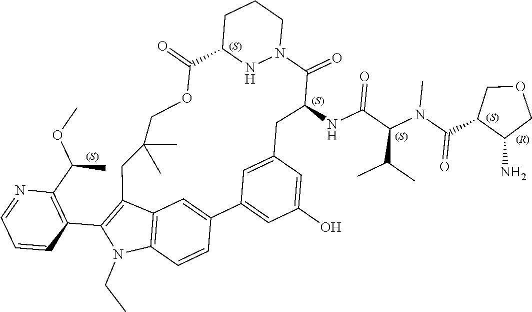 |
| AA419 | 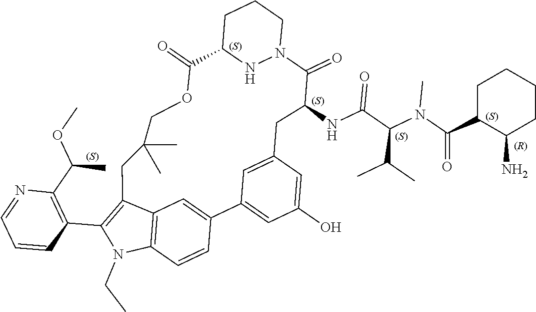 |
| AA420 | 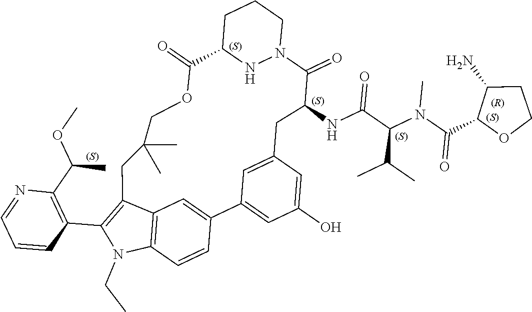 |
| AA421 | 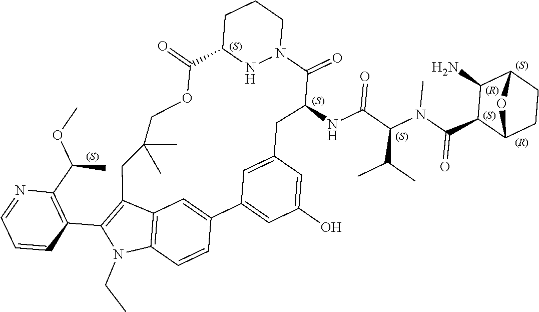 |
| AA422 | 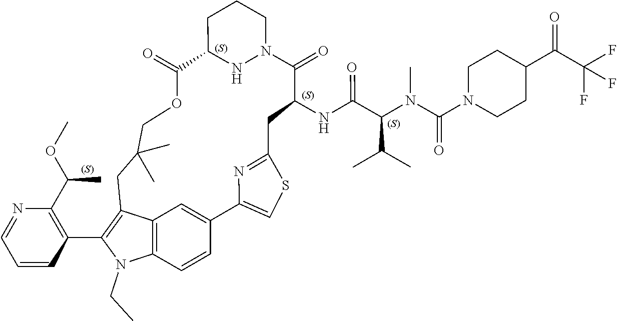 |
| AA423 | 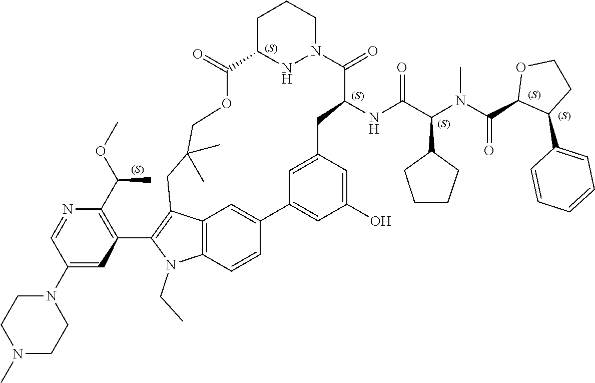 |
| AA424 | 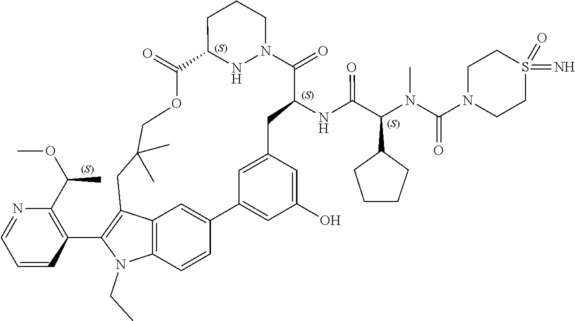 |
| AA425 | 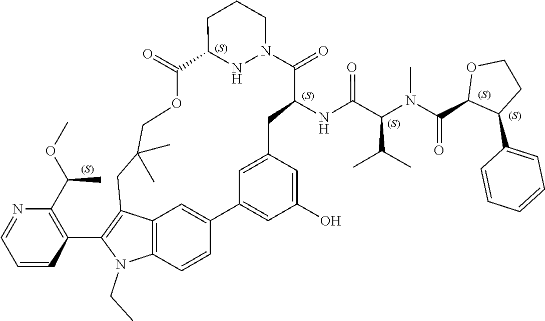 |
| AA426 | 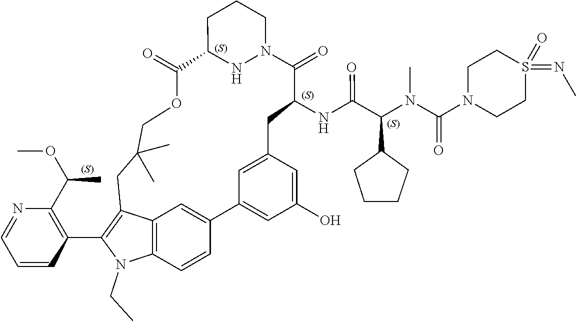 |
| AA427 | 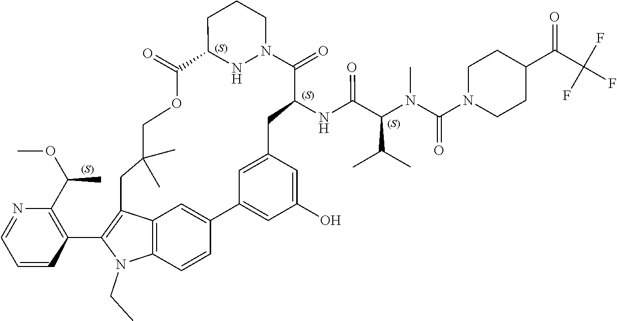 |
| AA428 | 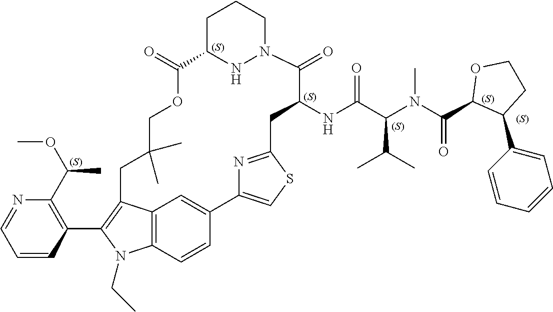 |
| AA429 | 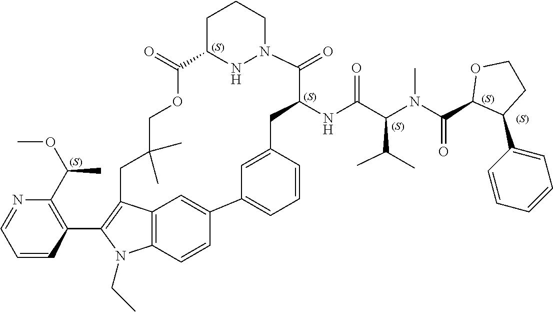 |
| AA430 | 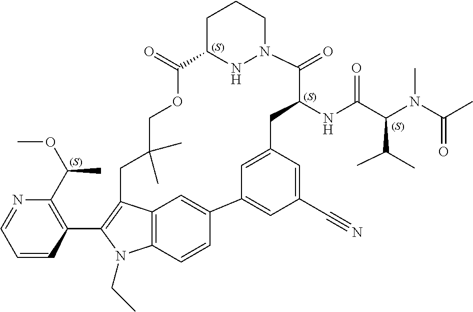 |
| AA431 | 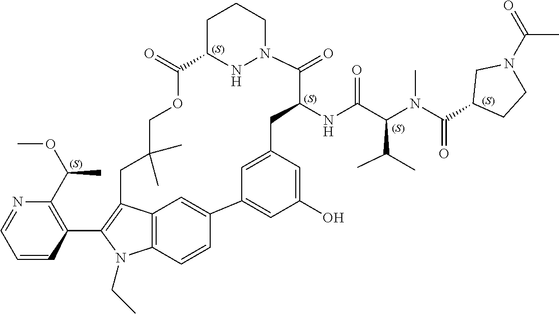 |
| AA432 | 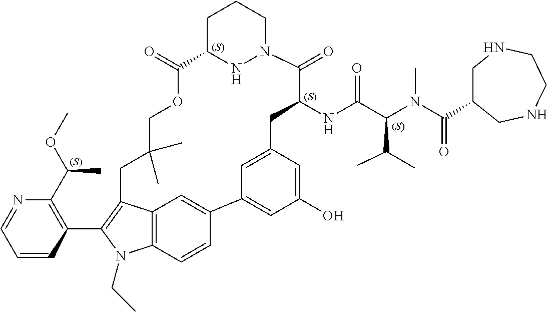 |
| AA433 | 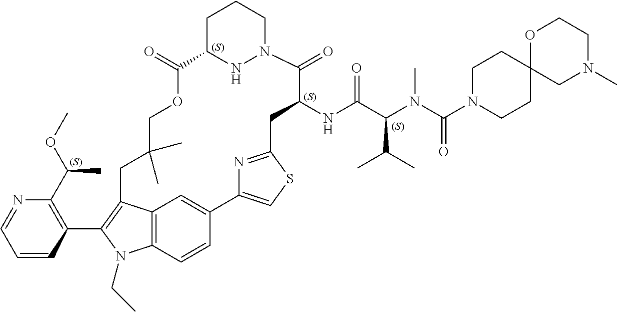 |
| AA434 | 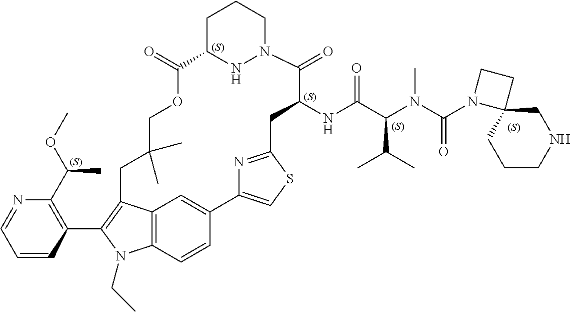 |
| AA435 | 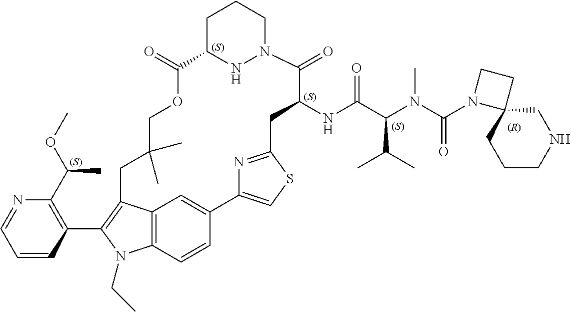 |
| AA436 | 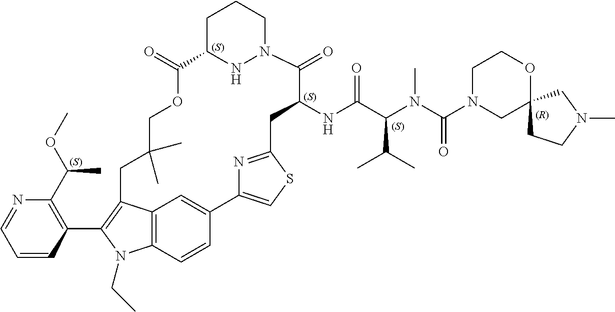 |
| AA437 | 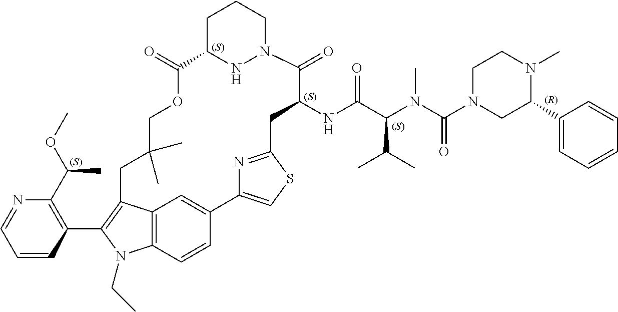 |
| AA438 | 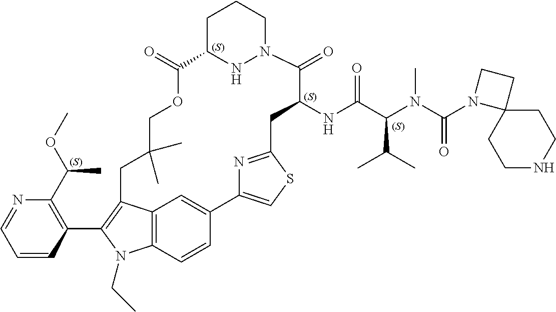 |
| AA439 | 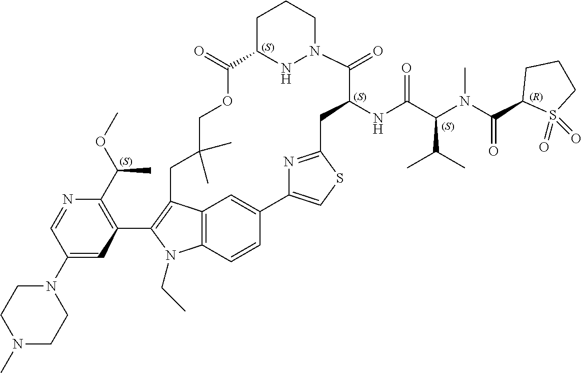 |
| AA440 | 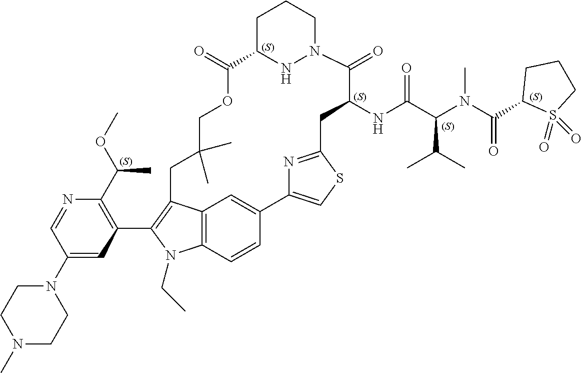 |
| AA441 | 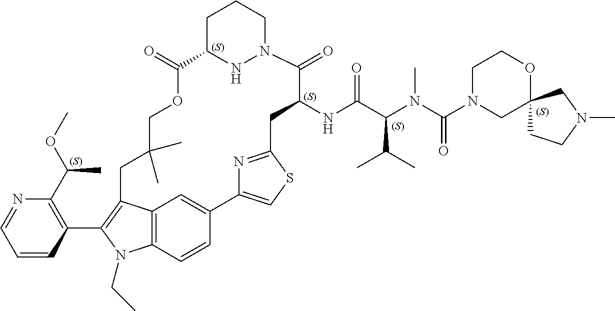 |
| AA442 | 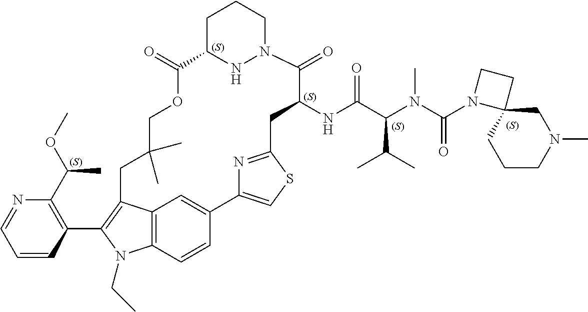 |
| AA443 | 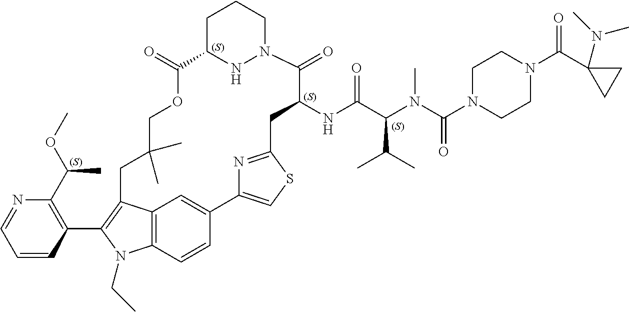 |
| AA444 | 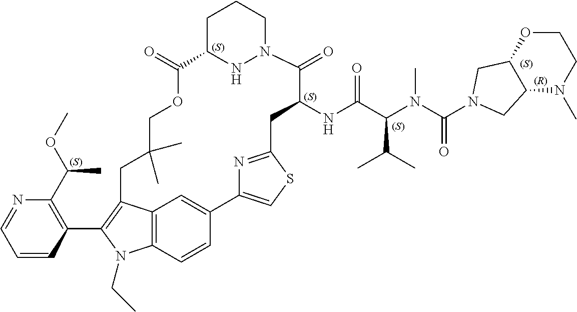 |
| AA445 | 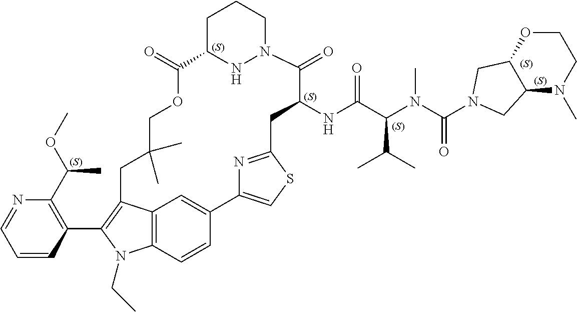 |
| AA446 | 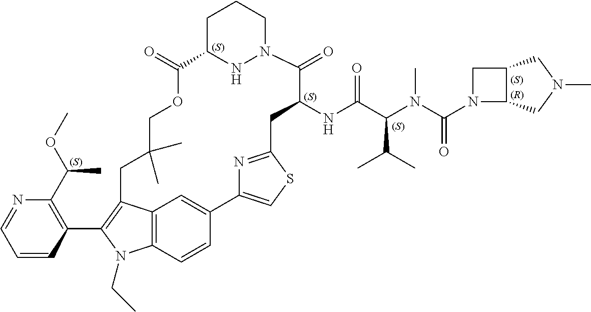 |
| AA447 | 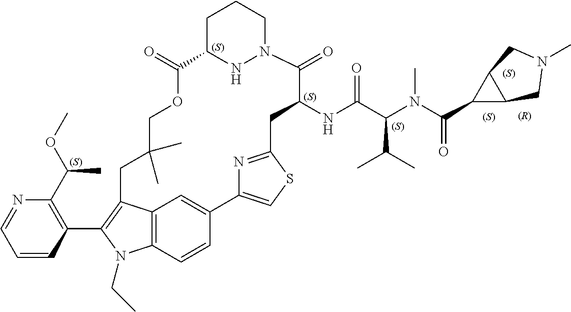 |
| AA448 | 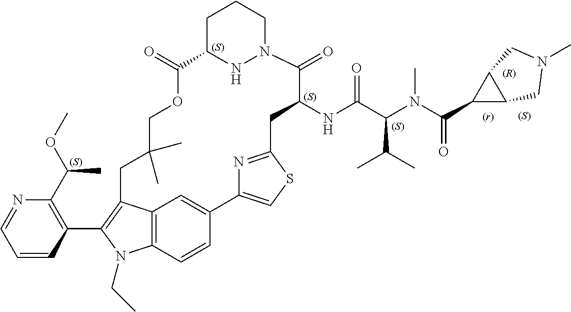 |
| AA449 | 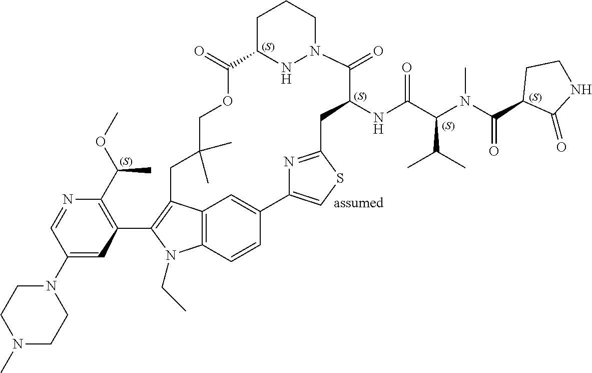 |
| AA450 | 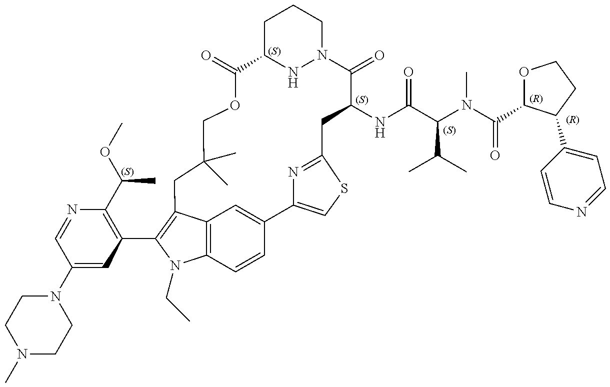 |
| AA451 | 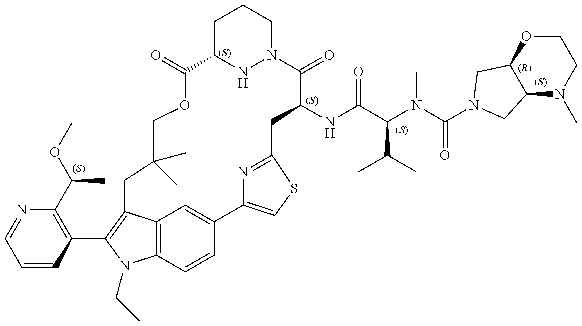 |
| AA452 | 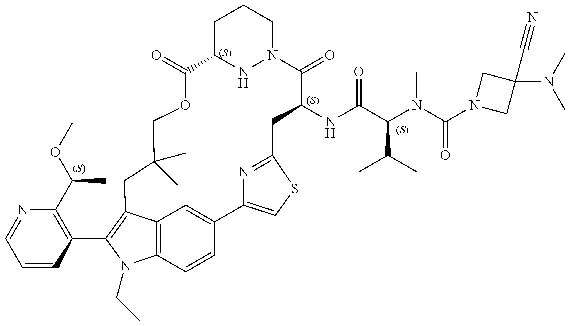 |
| AA453 | 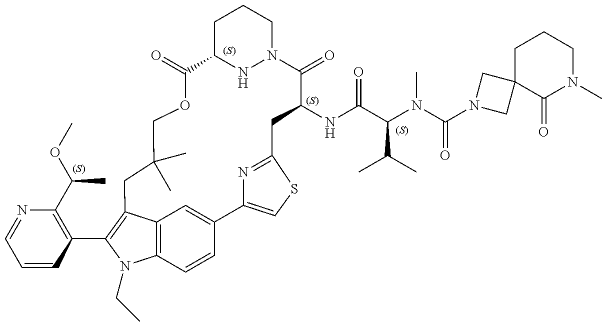 |
| AA454 | 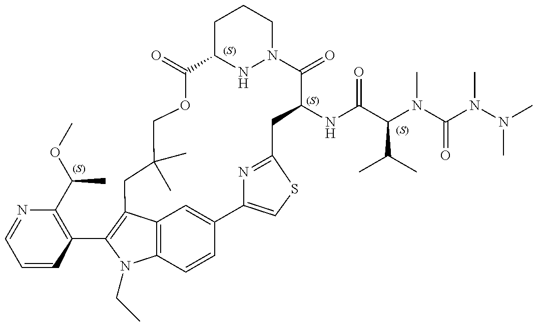 |
| AA455 | 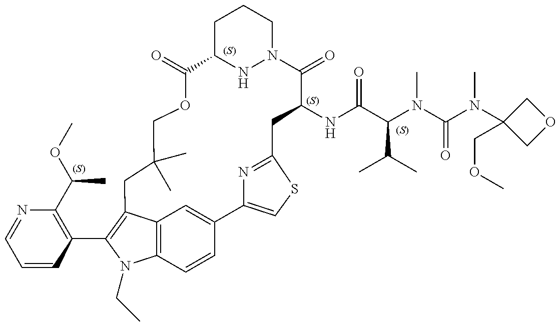 |
| AA456 | 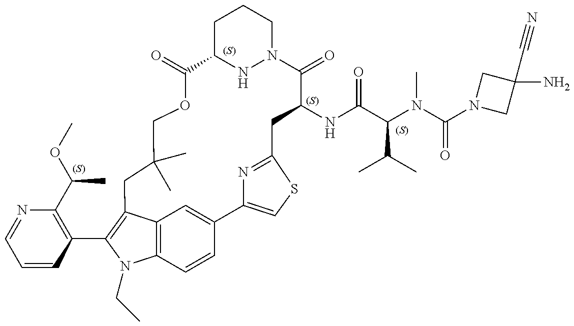 |
| AA457 | 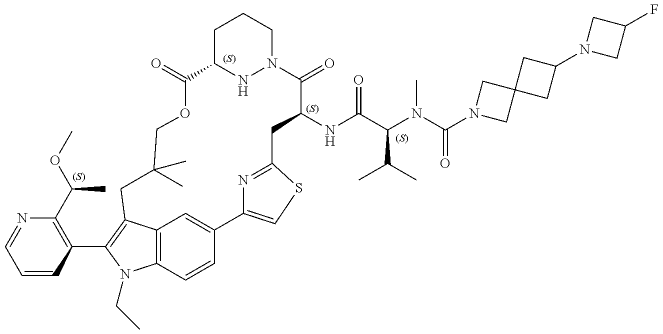 |
| AA458 | 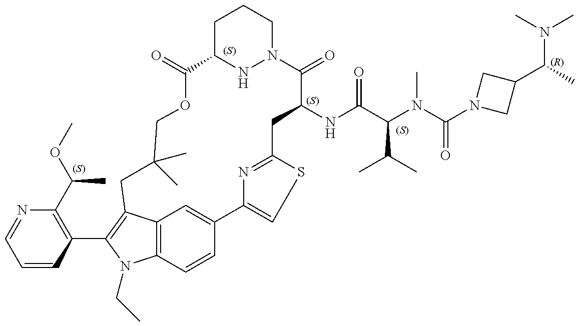 |
| AA459 | 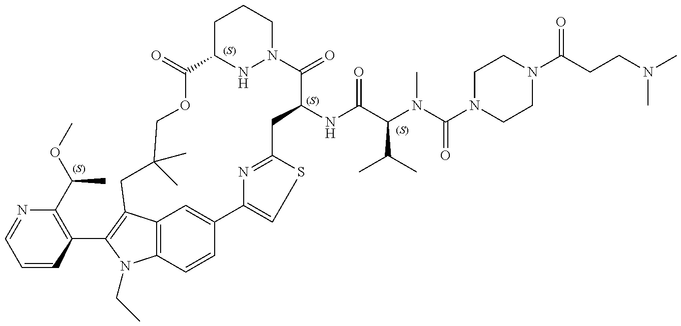 |
| AA460 | 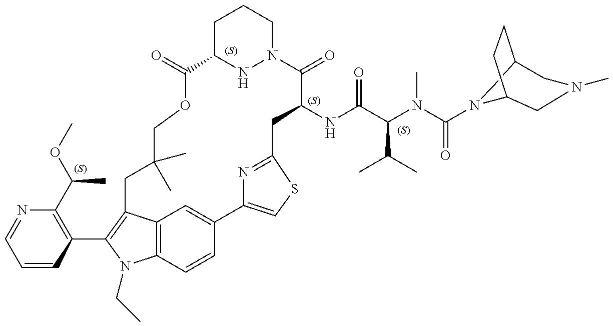 |
| AA461 | 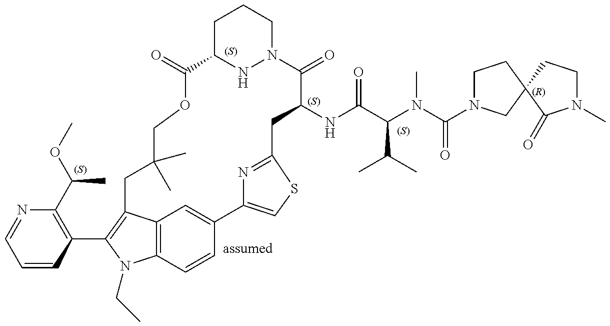 |
| AA462 | 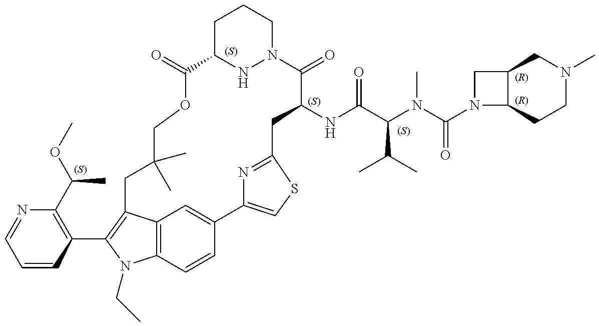 |
| AA463 | 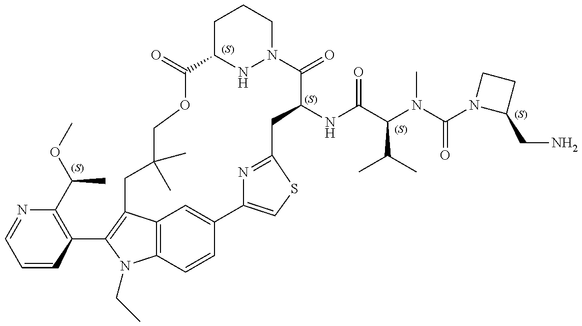 |
| AA464 | 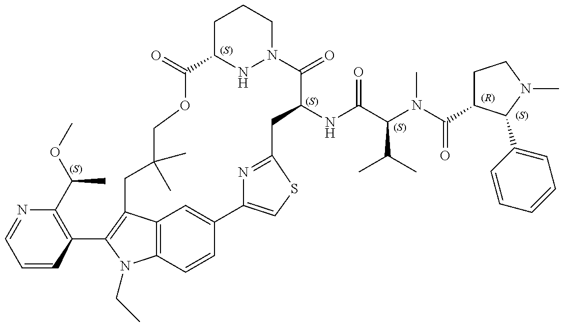 |
| AA465 | 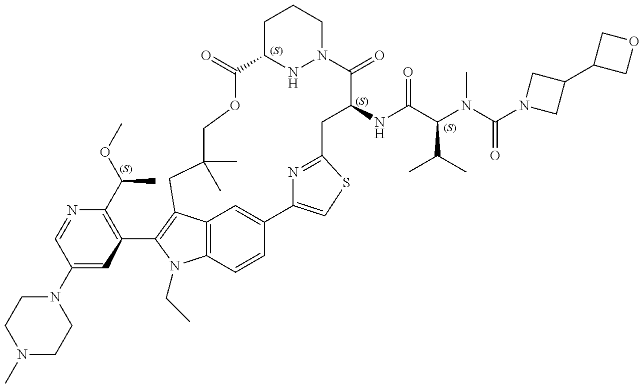 |
| AA466 | 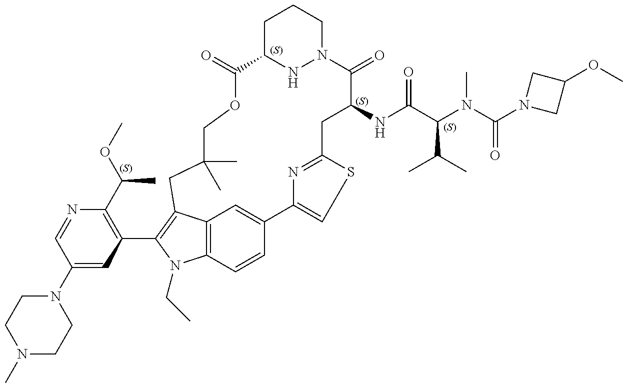 |
| AA467 | 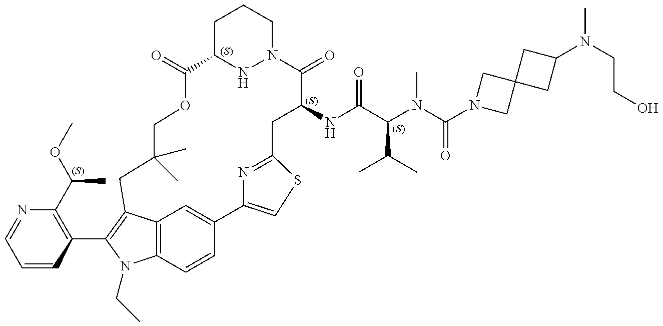 |
| AA468 | 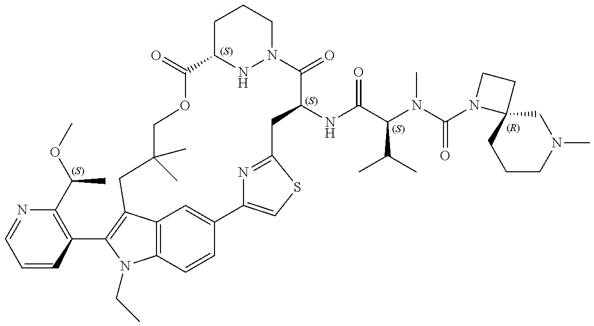 |
| AA469 | 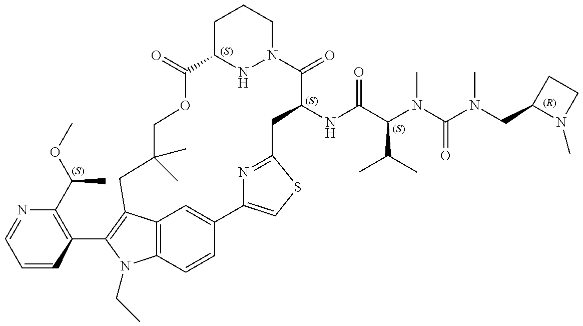 |
| AA470 | 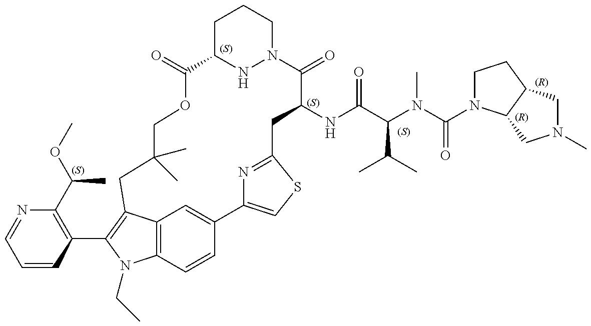 |
| AA471 | 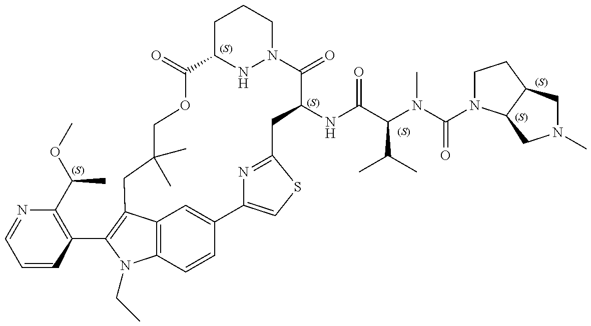 |
| AA472 | 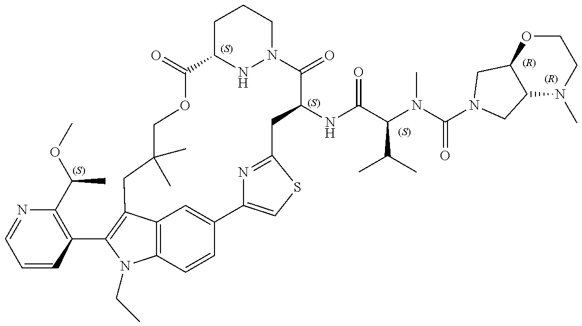 |
| AA473 | 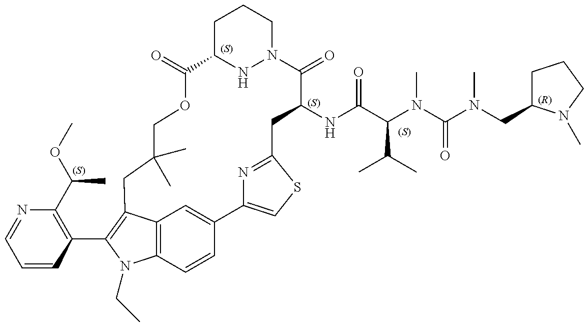 |
| AA474 | 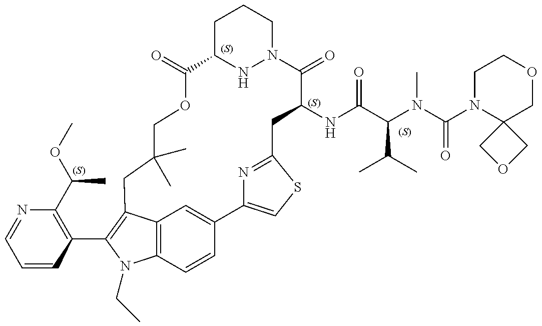 |
| AA475 | 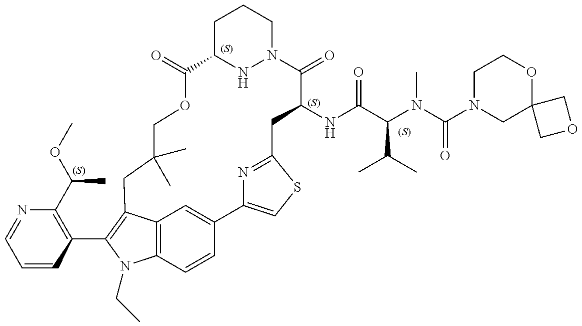 |
| AA476 | 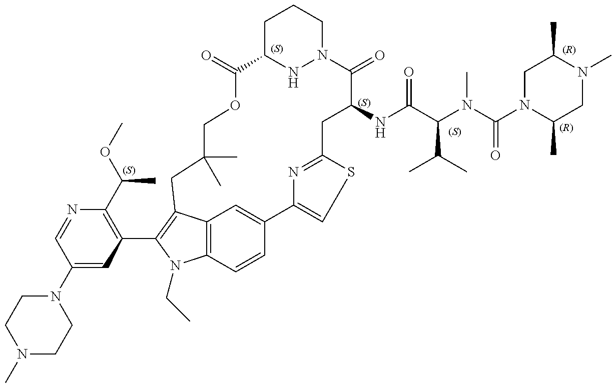 |
| AA477 | 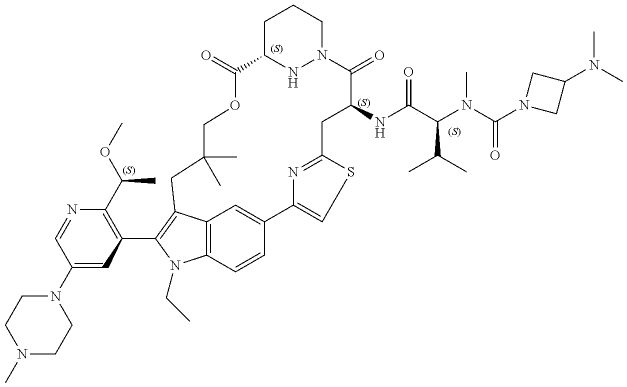 |
| AA478 | 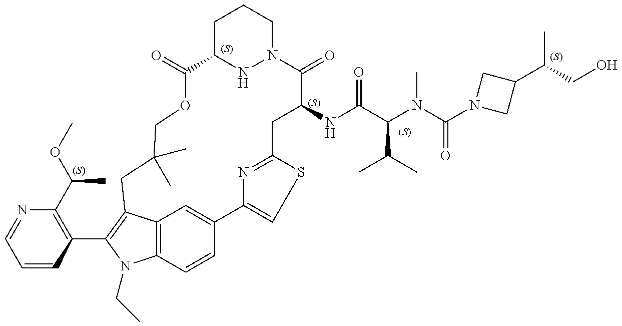 |
| AA479 | 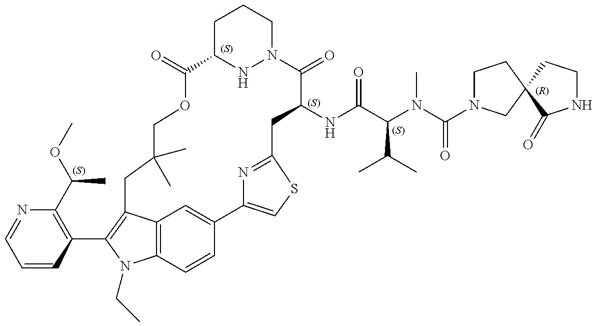 |
| AA480 | 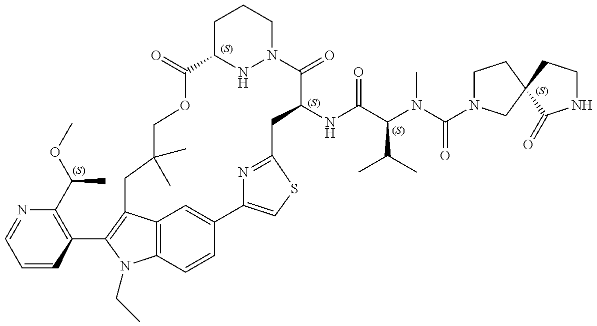 |
| AA481 | 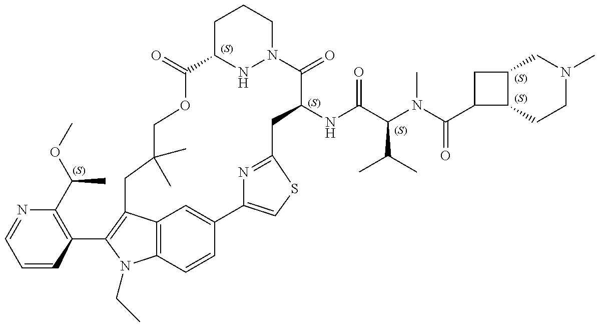 |
| AA482 | 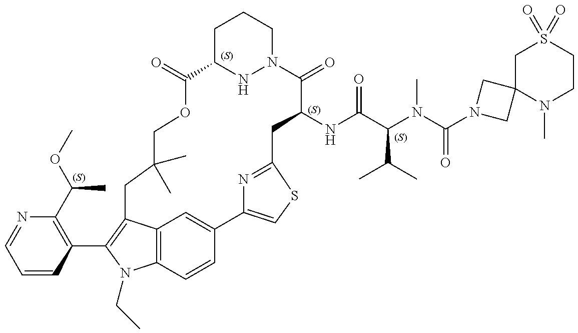 |
| AA483 | 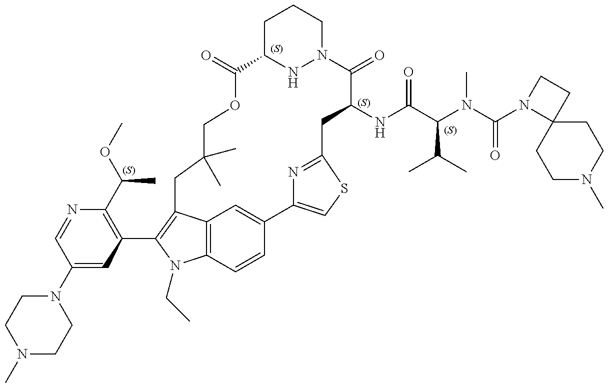 |
| AA484 | 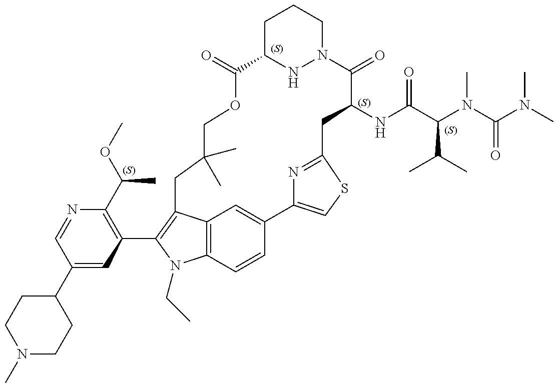 |
| AA485 | 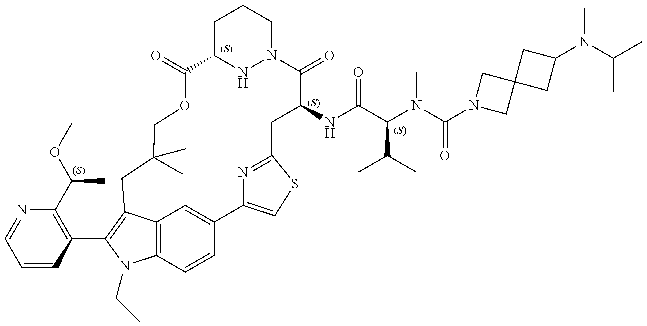 |
| AA486 | 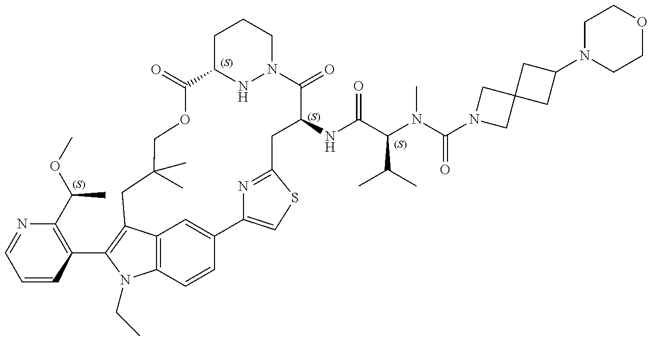 |
| AA487 | 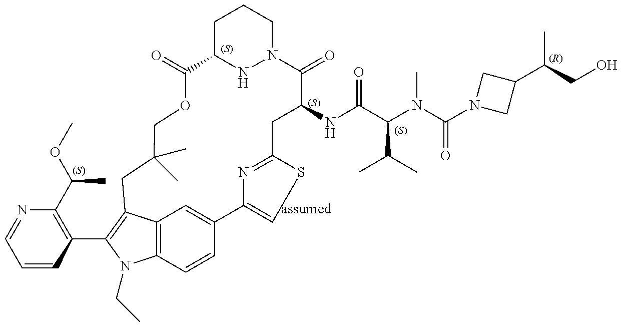 |
| AA488 | 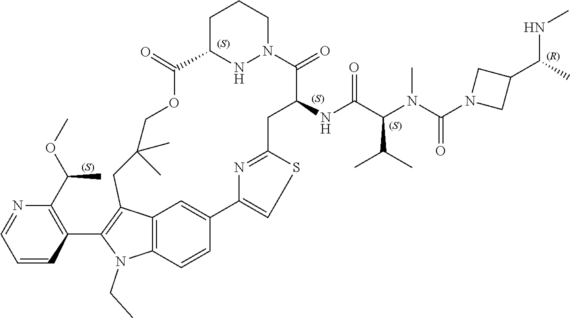 |
| AA489 | 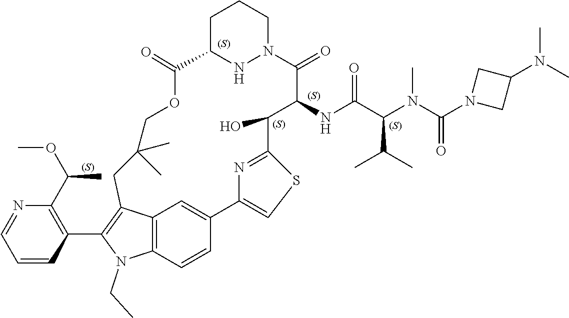 |
| AA490 | 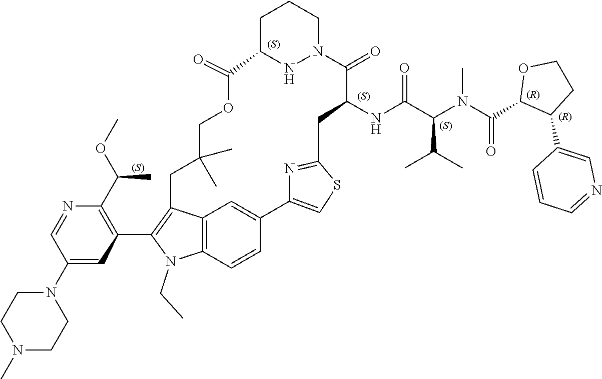 |
| AA491 | 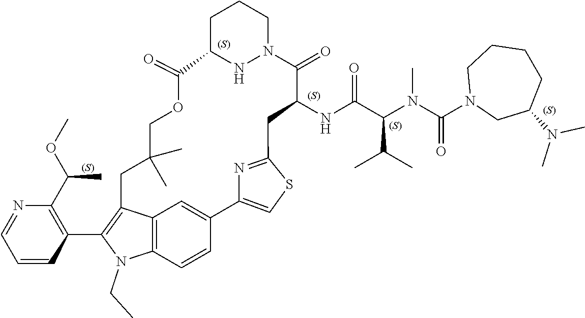 |
| AA492 | 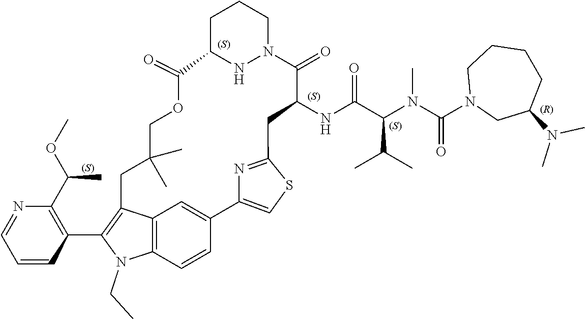 |
| AA493 | 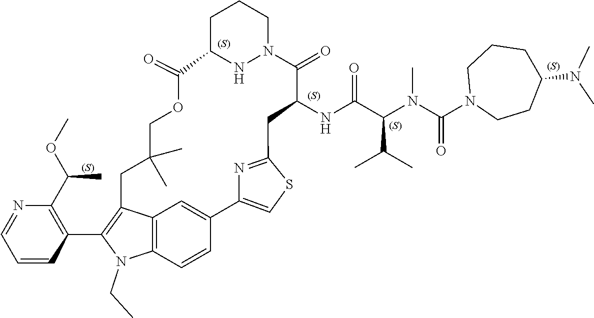 |
| AA494 | 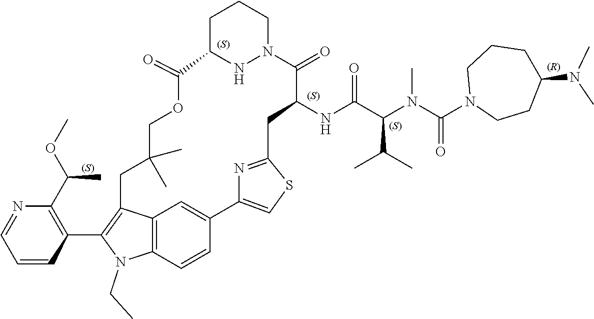 |
| AA495 | 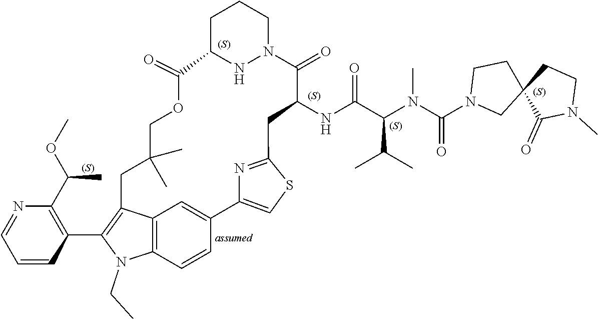 |
| AA496 | 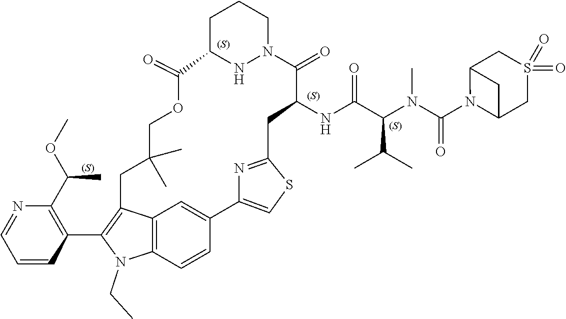 |
| AA497 | 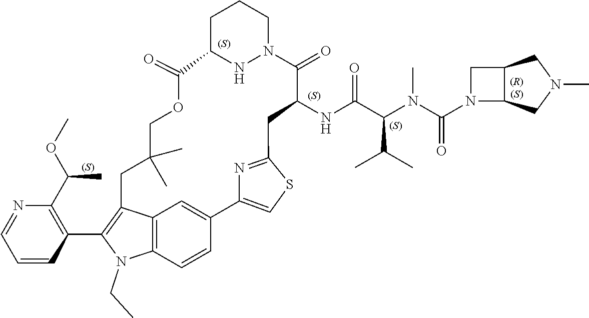 |
| AA498 | 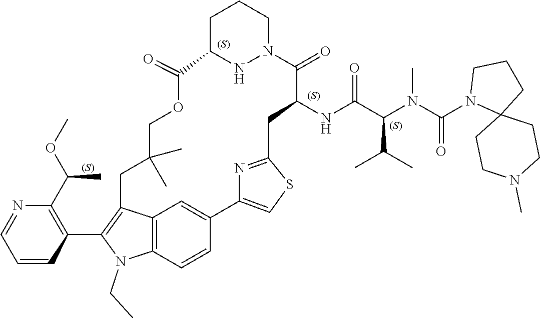 |
| AA499 | 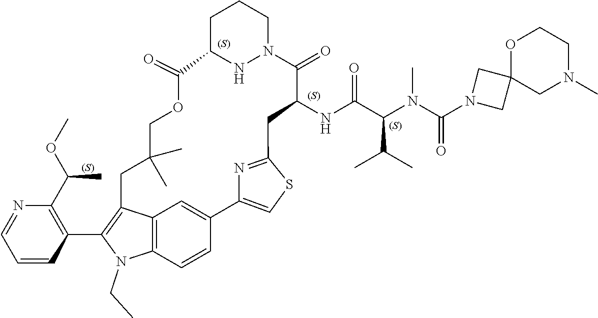 |
| AA500 | 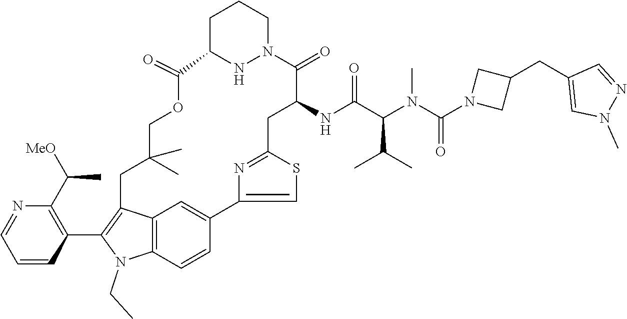 |
| AA501 | 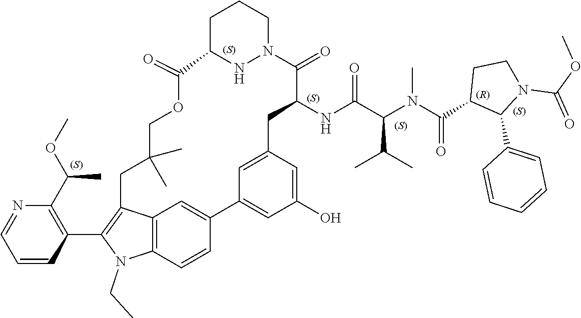 |
| AA502 | 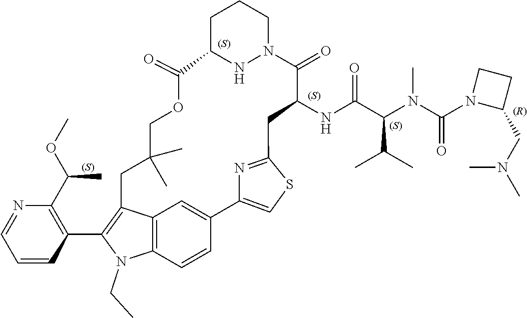 |
| AA503 | 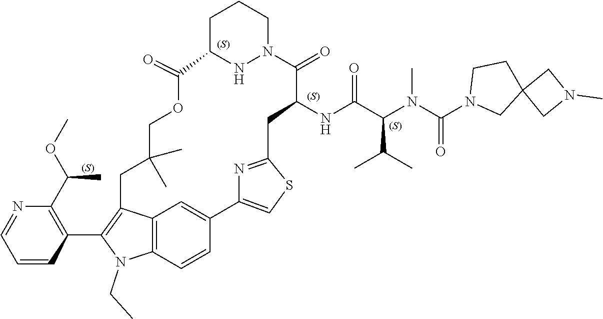 |
| AA504 | 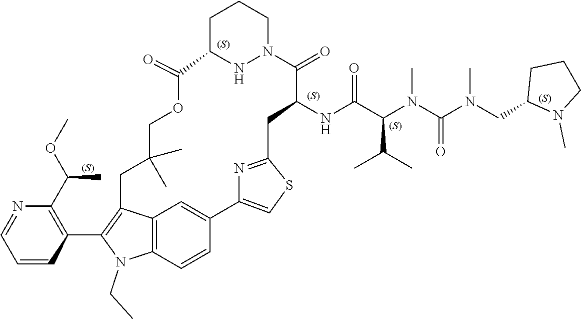 |
| AA505 | 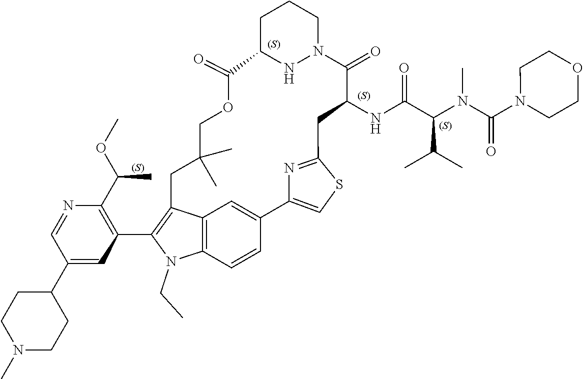 |
| AA506 | 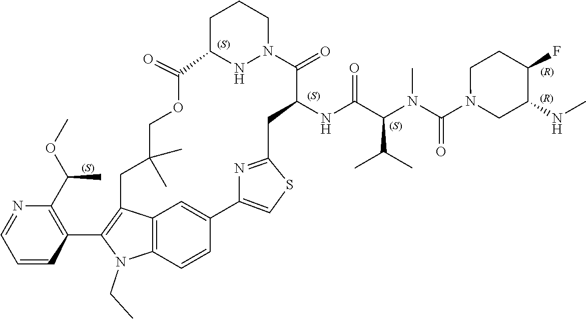 |
| AA507 | 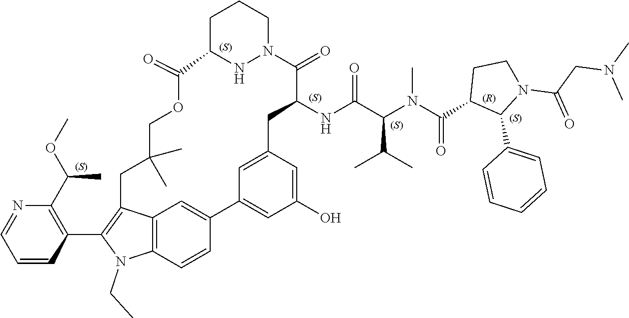 |
| AA508 | 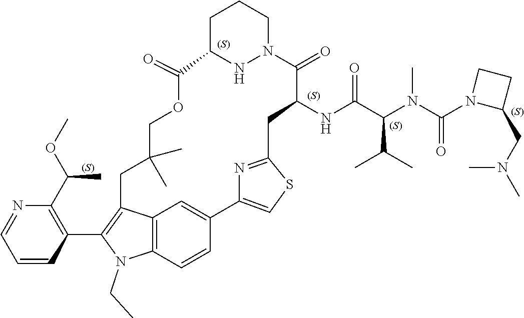 |
| AA509 | 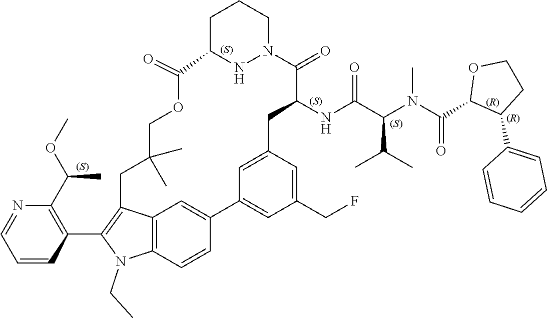 |
| AA510 | 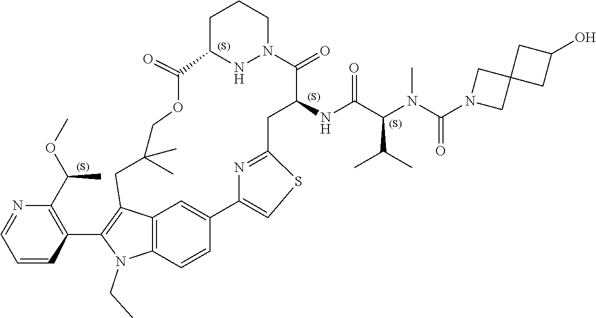 |
| AA511 | 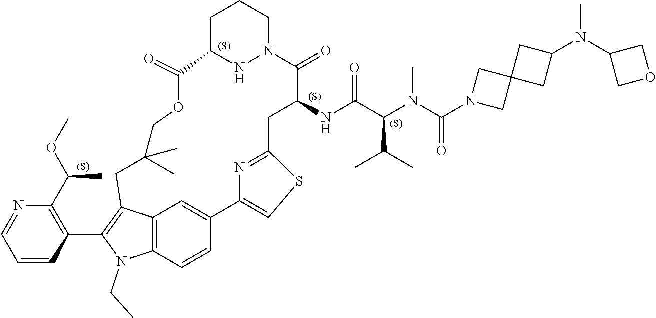 |
| AA512 | 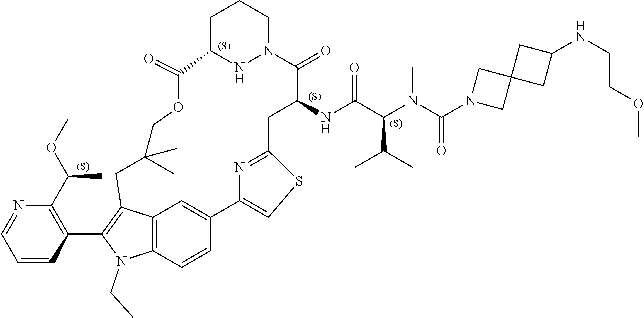 |
| AA513 | 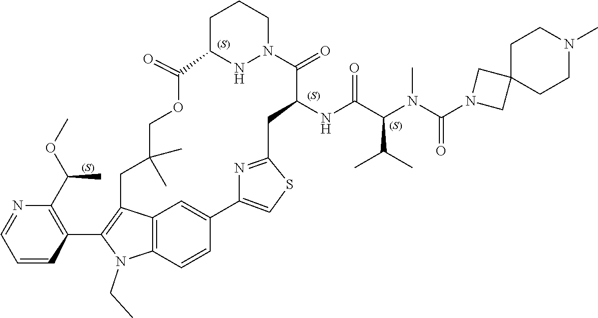 |
| AA514 | 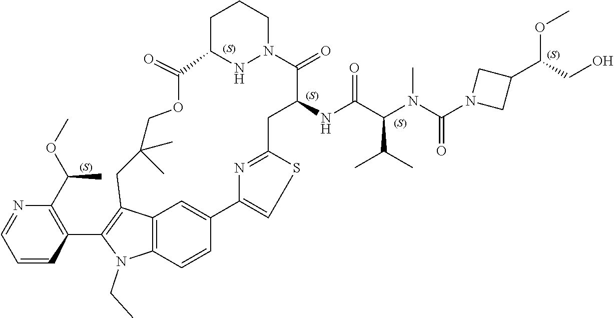 |
| AA515 | 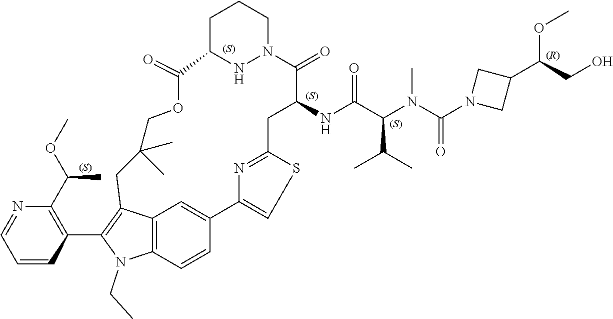 |
| AA516 | 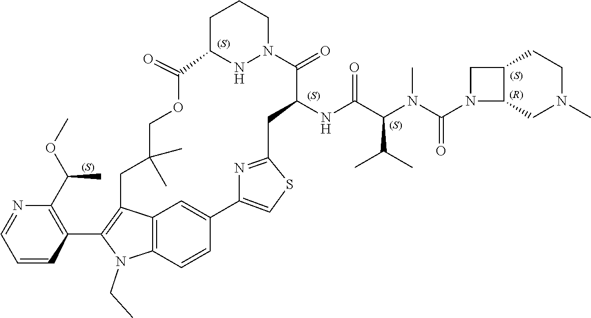 |
| AA517 | 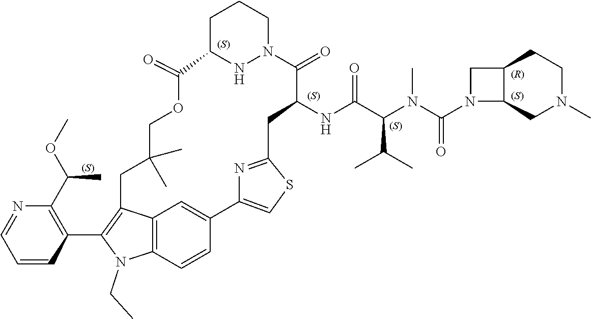 |
| AA518 | 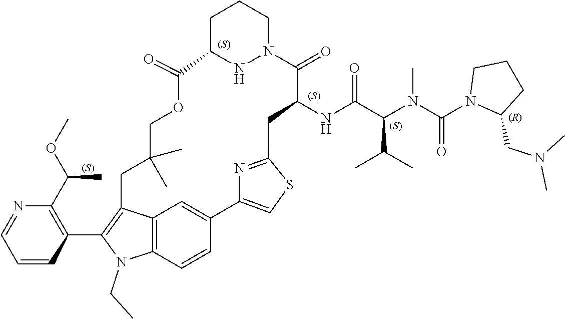 |
| AA519 | 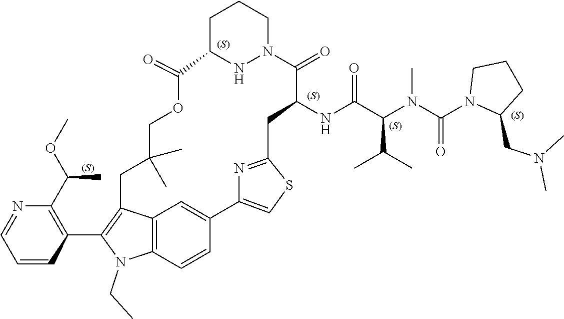 |
| AA520 | 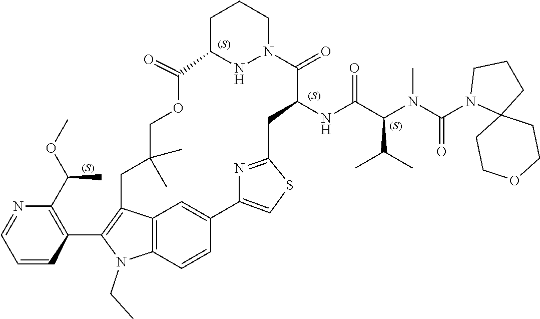 |
| AA521 | 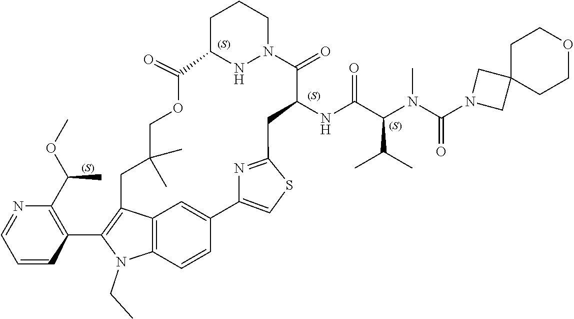 |
| AA522 | 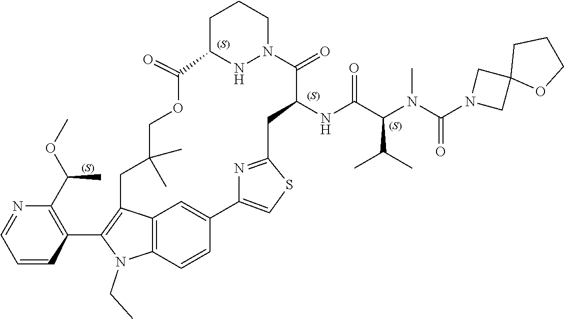 |
| AA523 | 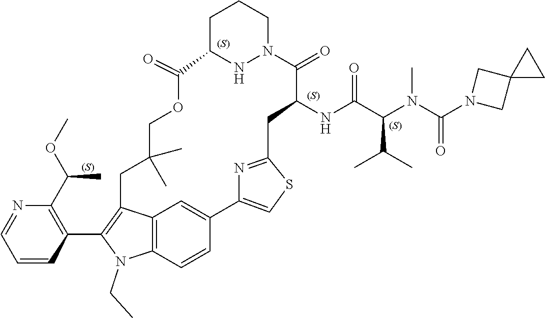 |
| AA524 | 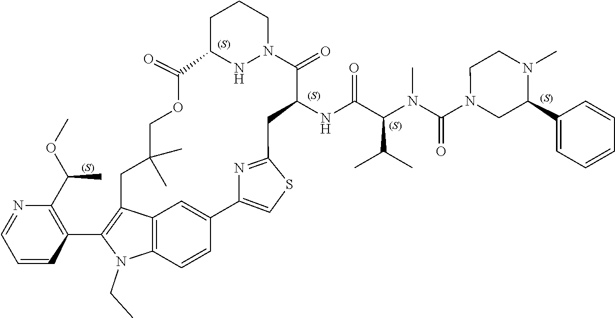 |
| AA525 | 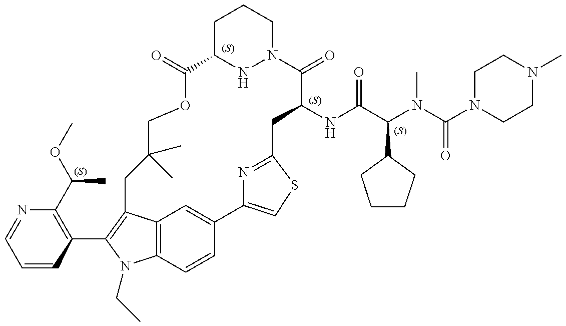 |
| AA526 | 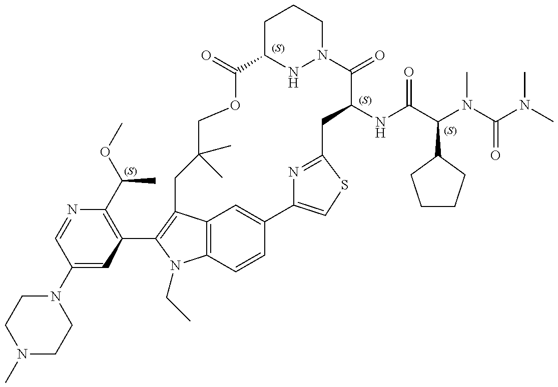 |
| AA527 | 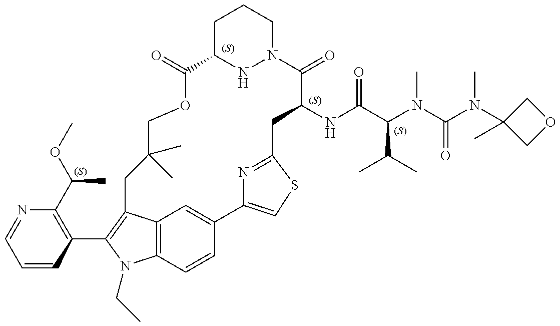 |
| AA528 | 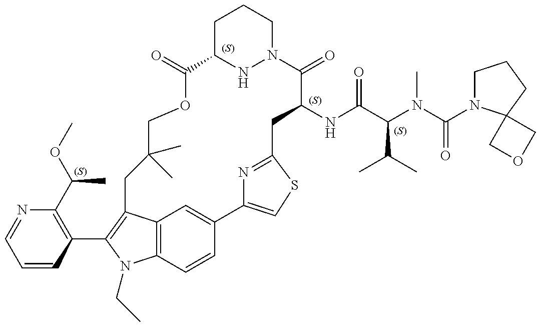 |
| AA529 | 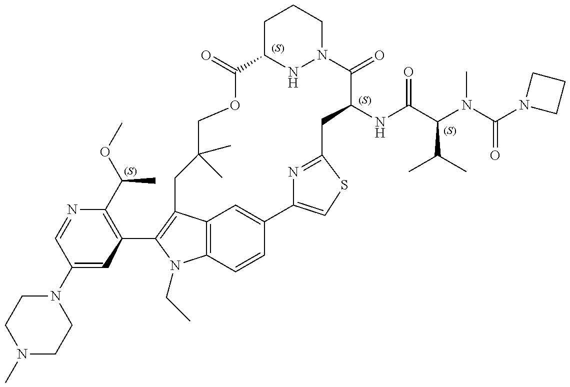 |
| AA530 | 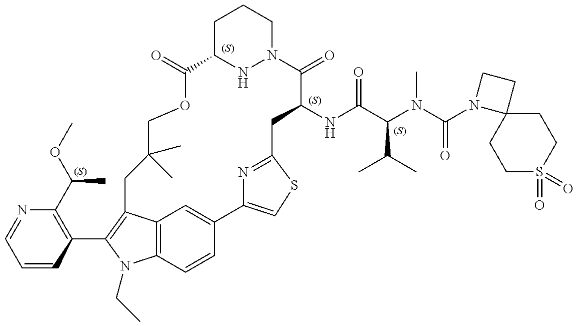 |
| AA531 | 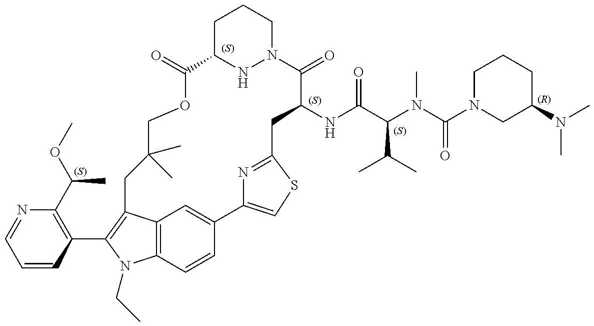 |
| AA532 | 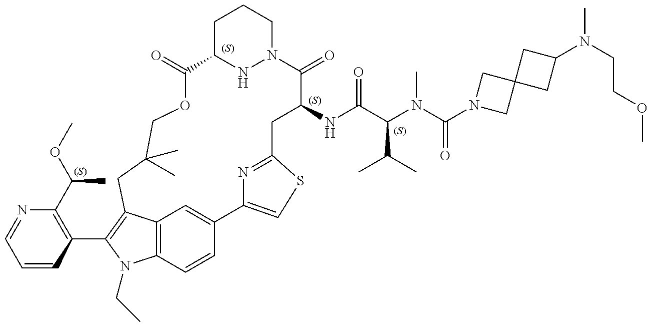 |
| AA533 | 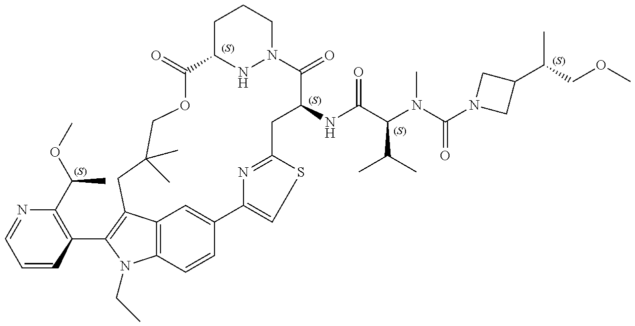 |
| AA534 | 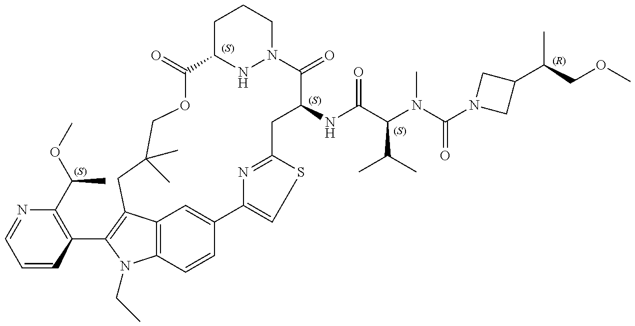 |
| AA535 | 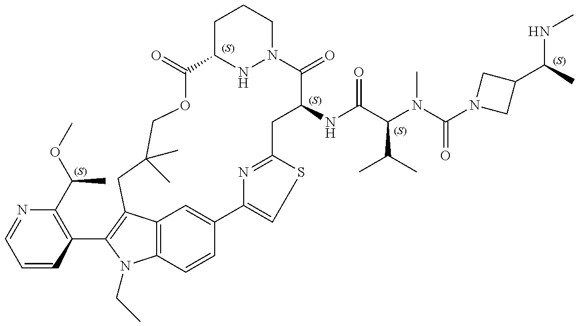 |
| AA536 | 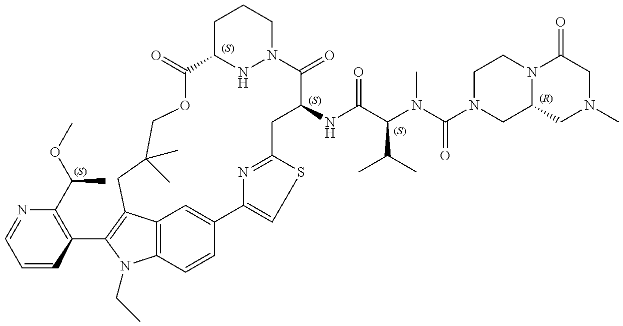 |
| AA537 | 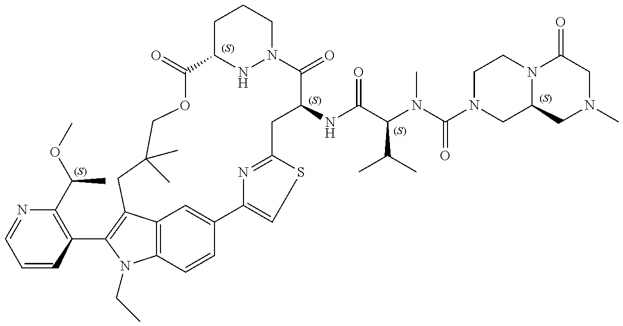 |
| AA538 | 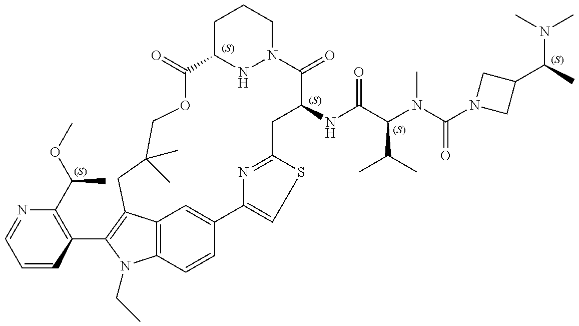 |
| AA539 | 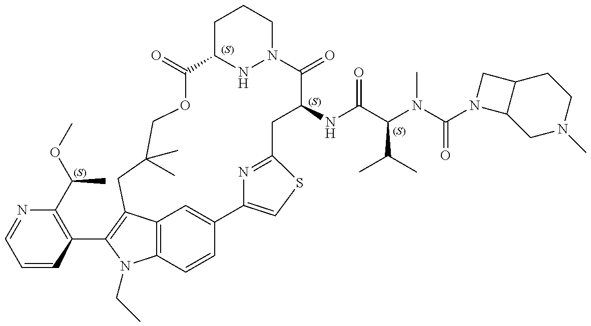 |
| AA540 | 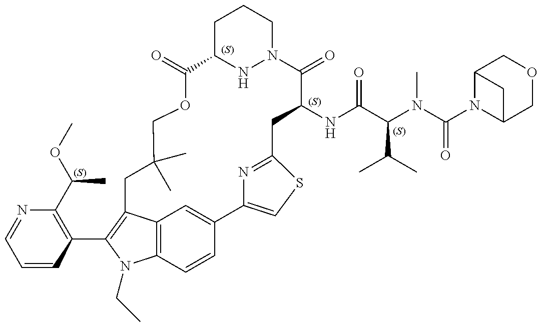 |
| AA541 | 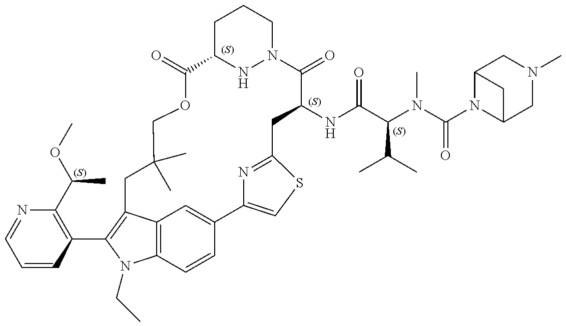 |
| AA542 | 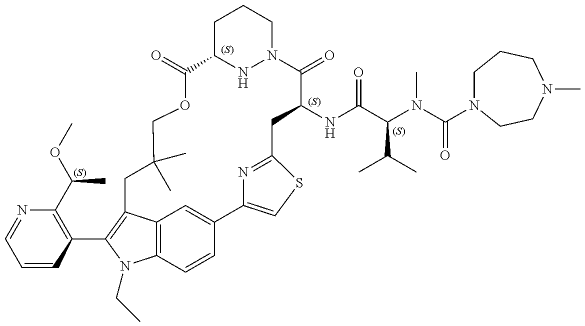 |
| AA543 | 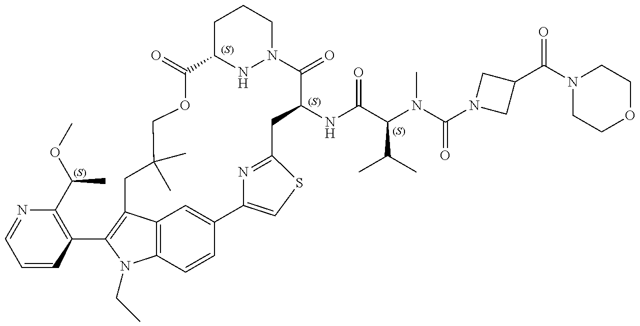 |
| AA544 | 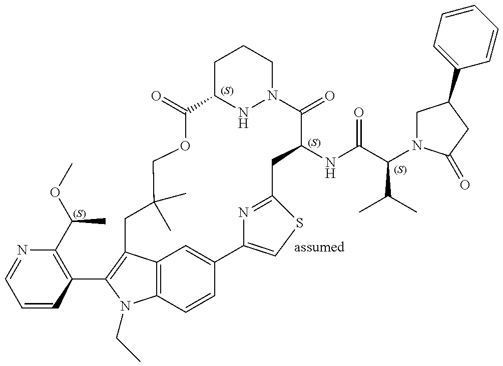 |
| AA545 | 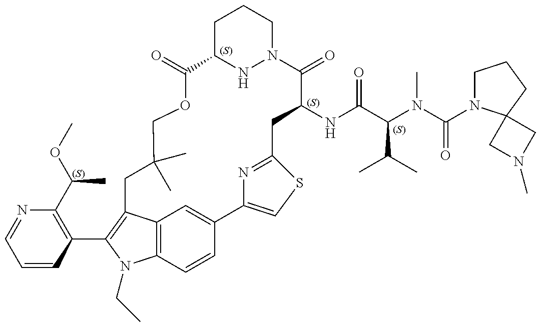 |
| AA546 | 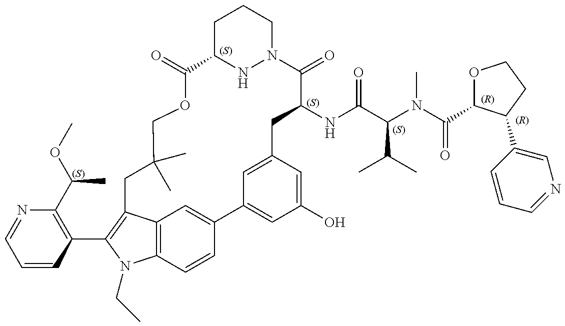 |
| AA547 | 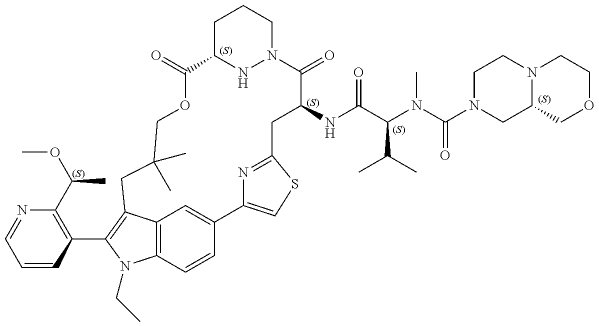 |
| AA548 | 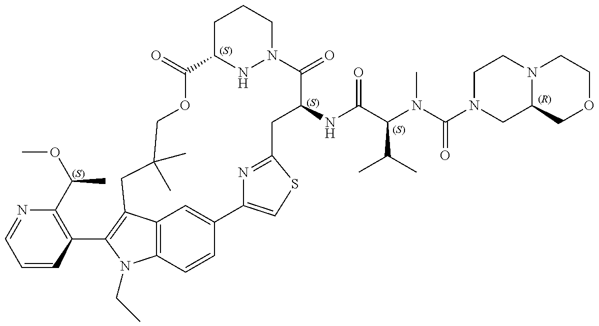 |
| AA549 | 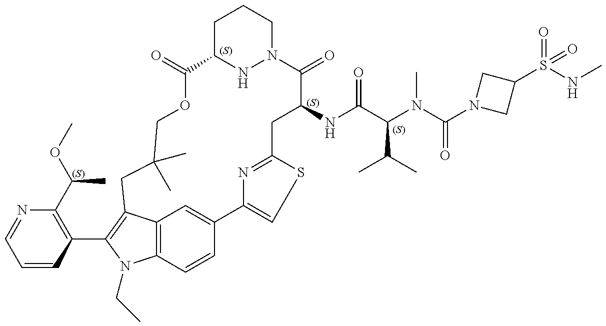 |
| AA550 | 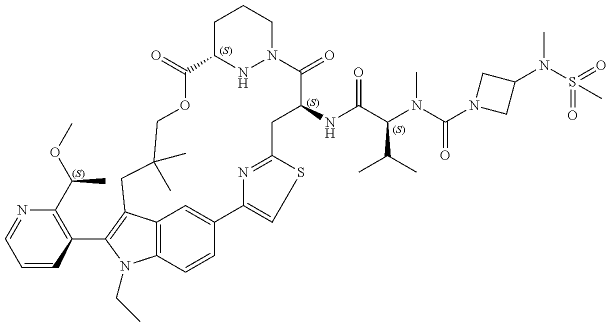 |
| AA551 | 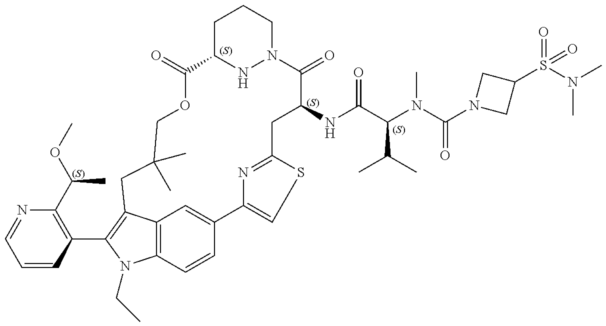 |
| AA552 | 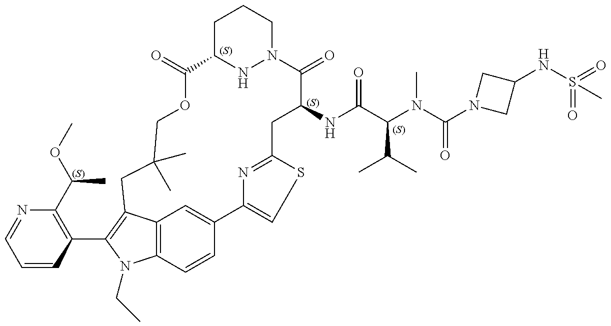 |
| AA553 | 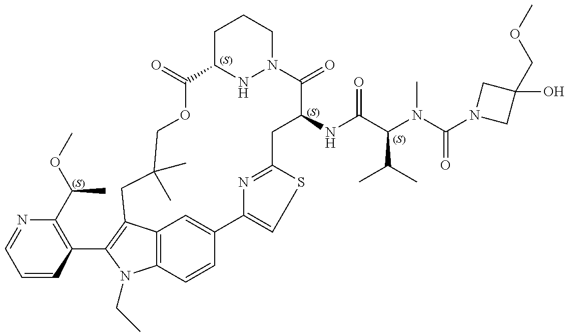 |
| AA554 | 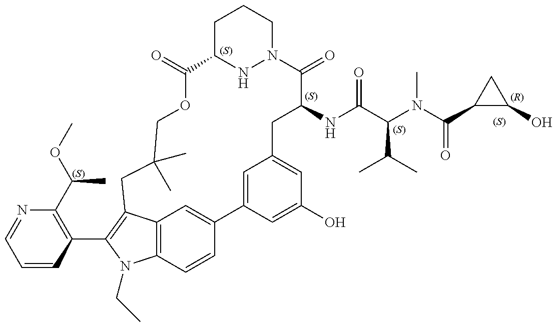 |
| AA555 | 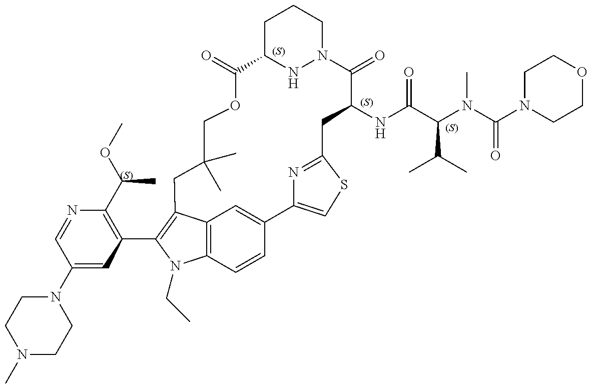 |
| AA556 | 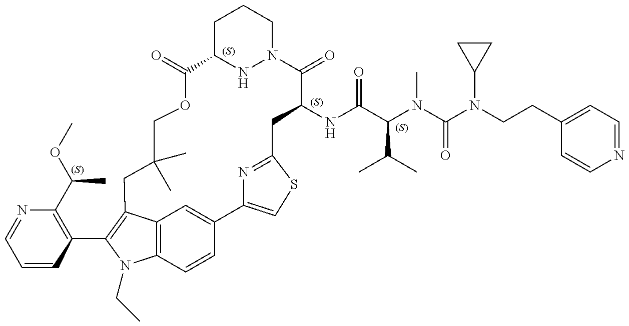 |
| AA557 | 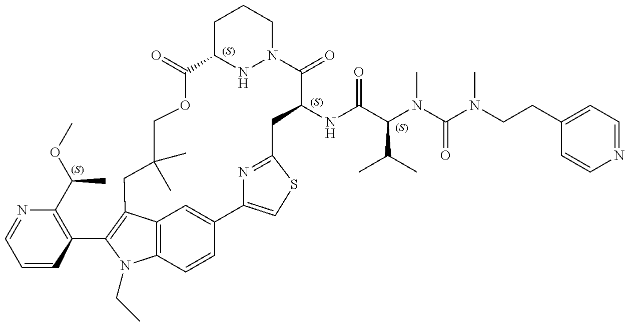 |
| AA558 | 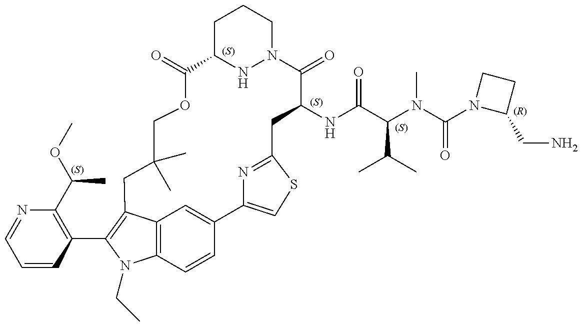 |
| AA559 | 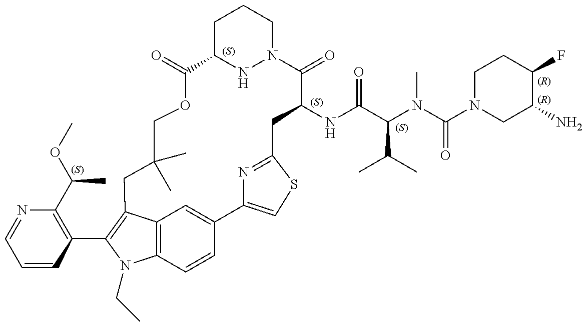 |
| AA560 | 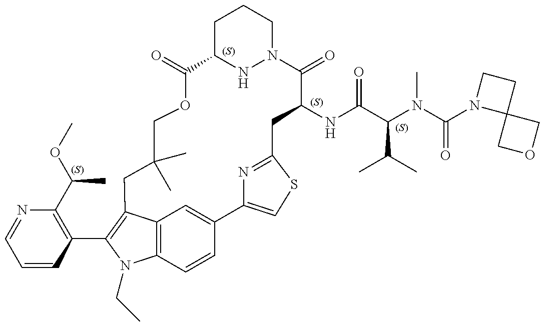 |
| AA561 | 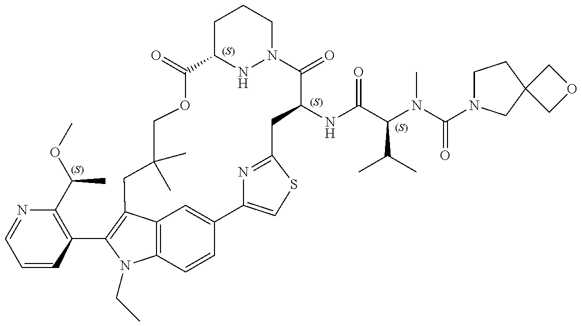 |
| AA562 | 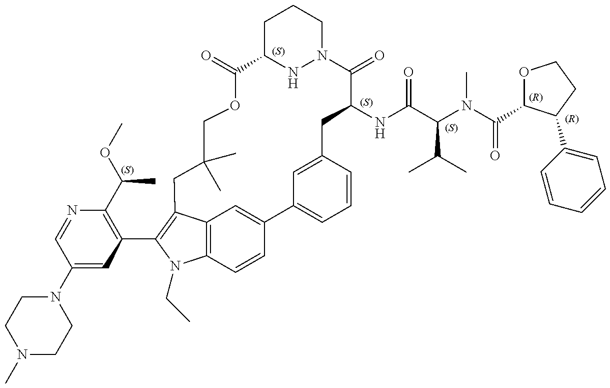 |
| AA563 | 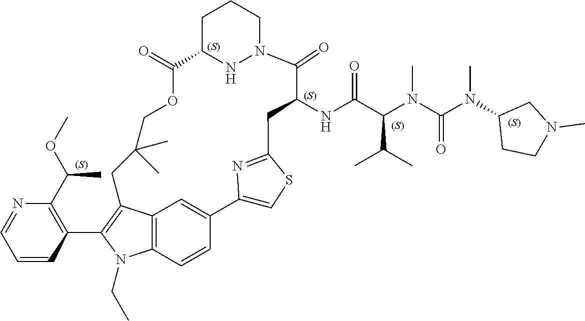 |
| AA564 | 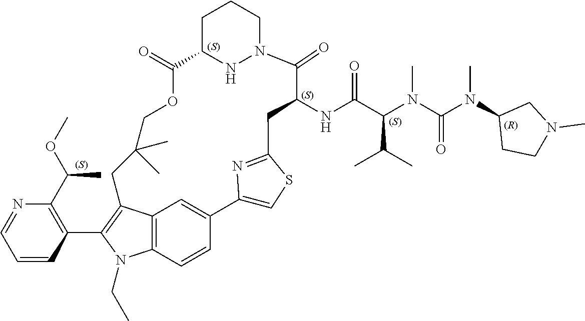 |
| AA565 | 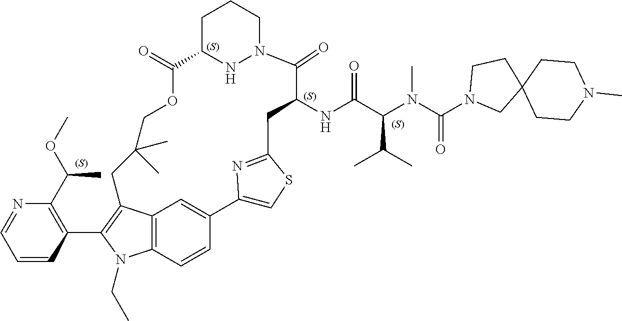 |
| AA566 | 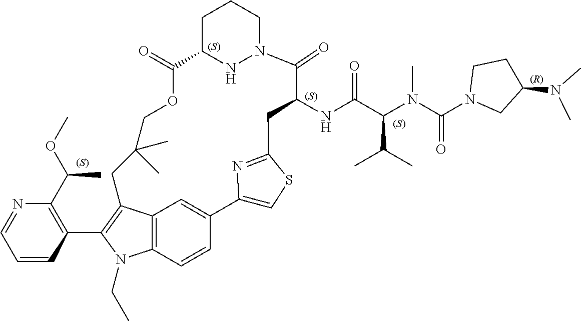 |
| AA567 | 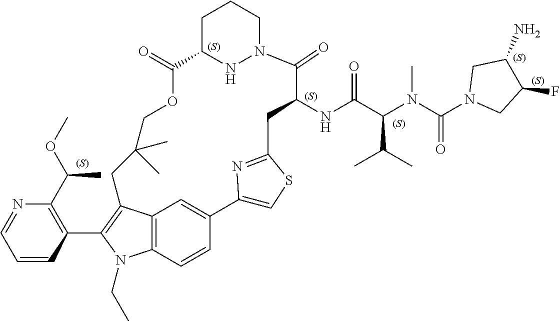 |
| AA568 | 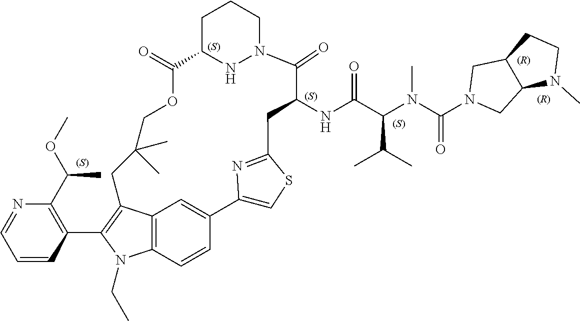 |
| AA569 | 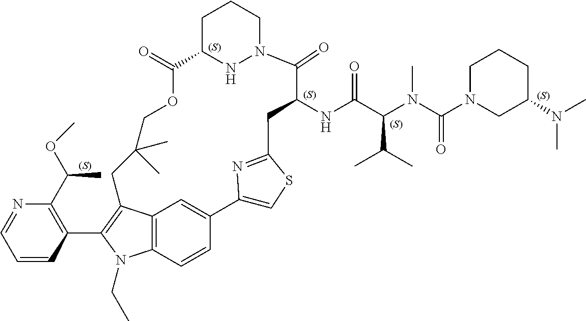 |
| AA570 | 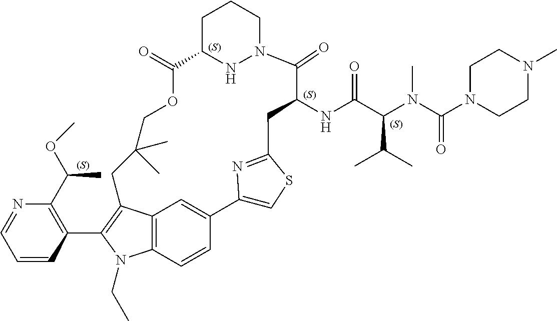 |
| AA571 | 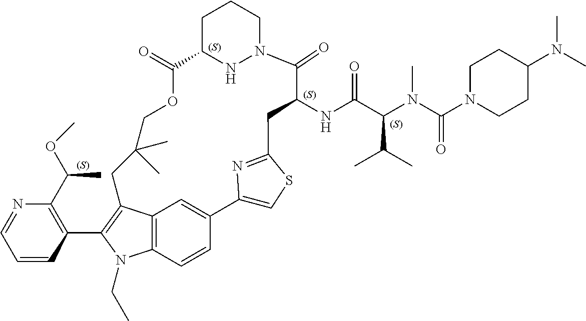 |
| AA572 | 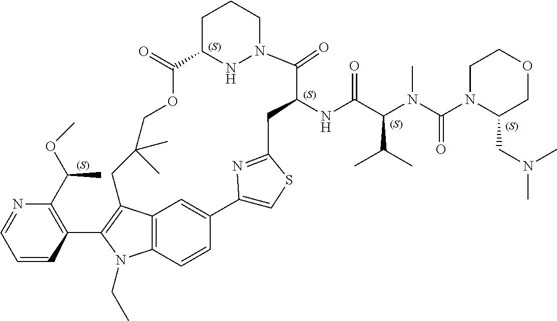 |
| AA573 | 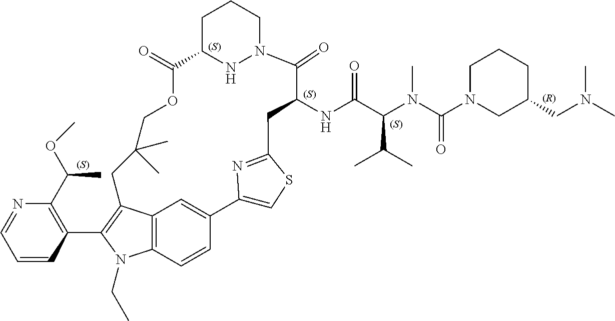 |
| AA574 | 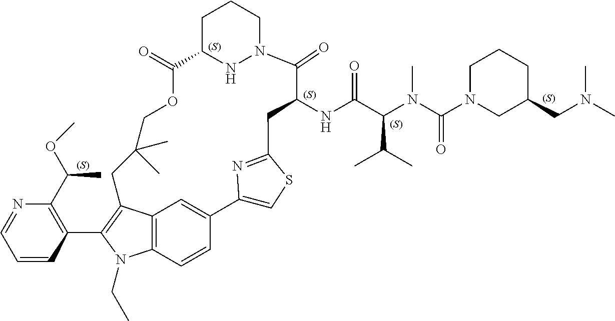 |
| AA575 | 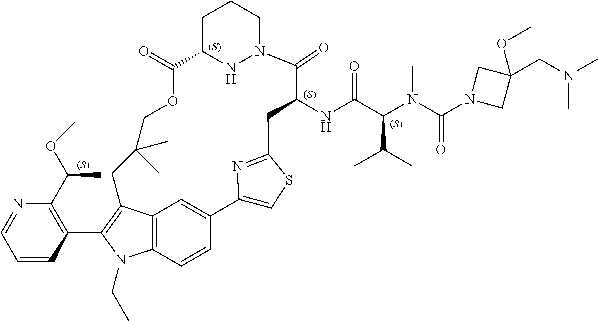 |
| AA576 | 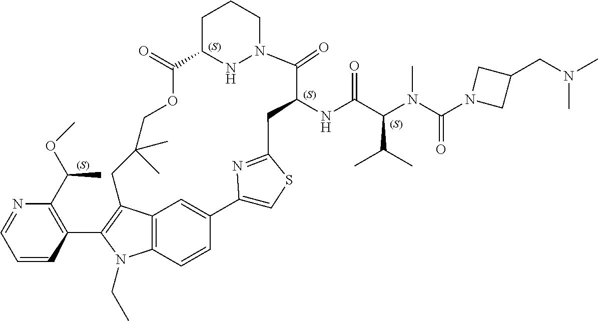 |
| AA577 | 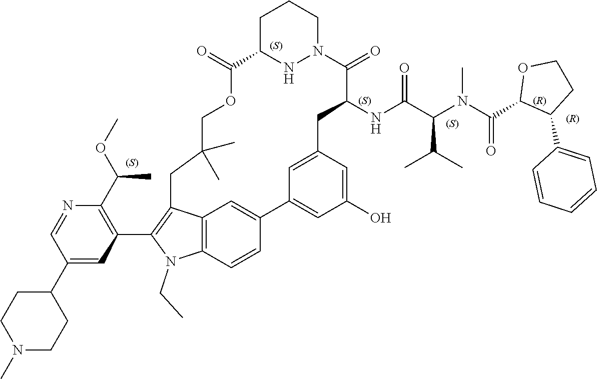 |
| AA578 | 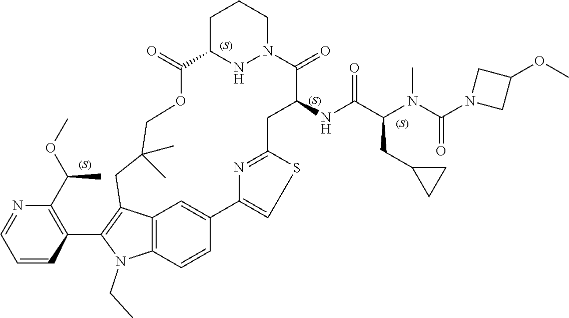 |
| AA579 | 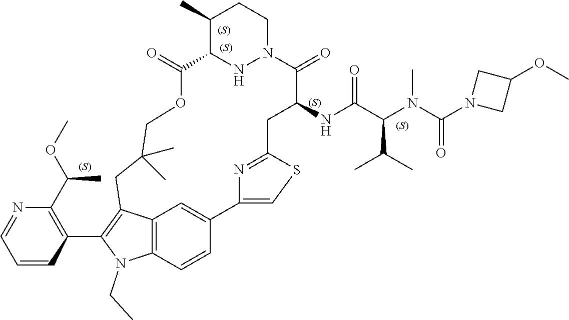 |
| AA580 | 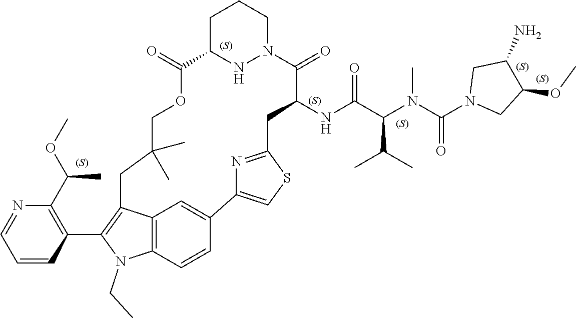 |
| AA581 | 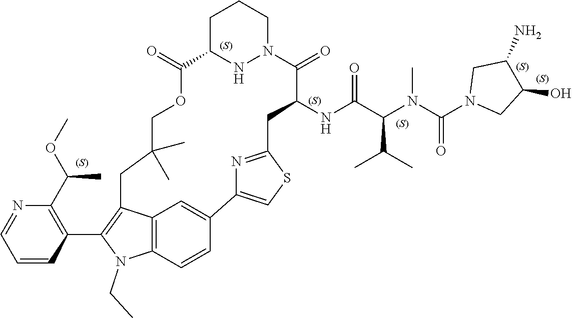 |
| AA582 | 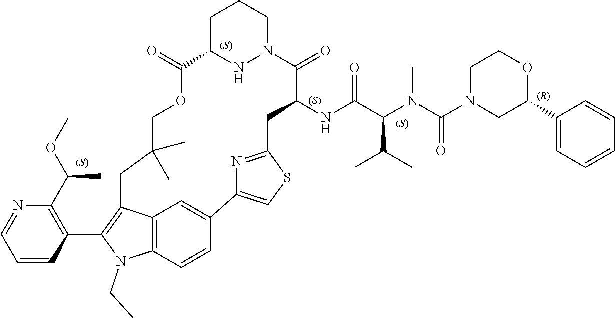 |
| AA583 | 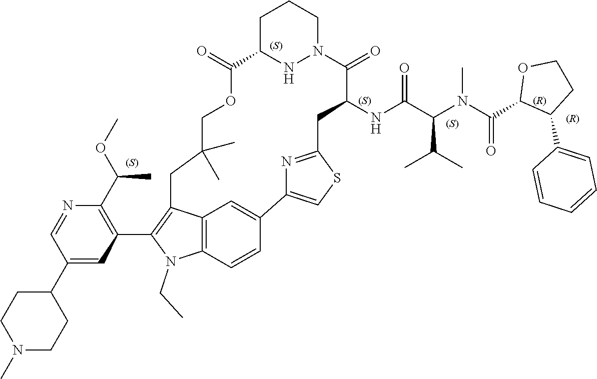 |
| AA584 | 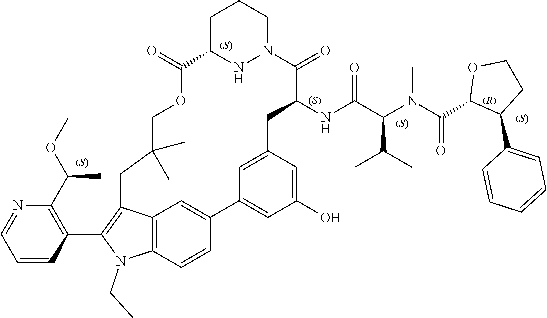 |
| AA585 | 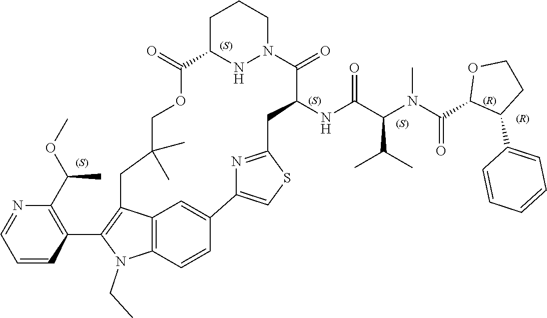 |
| AA586 | 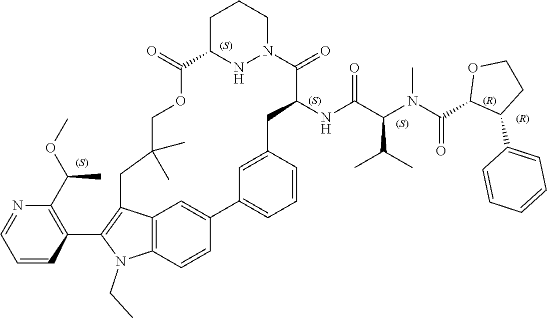 |
| AA587 | 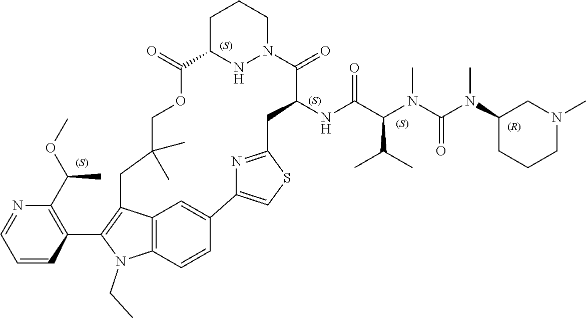 |
| AA588 | 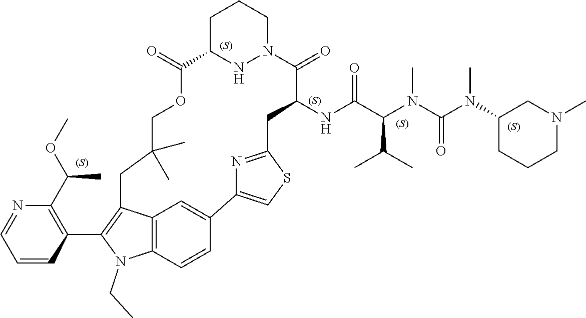 |
| AA589 | 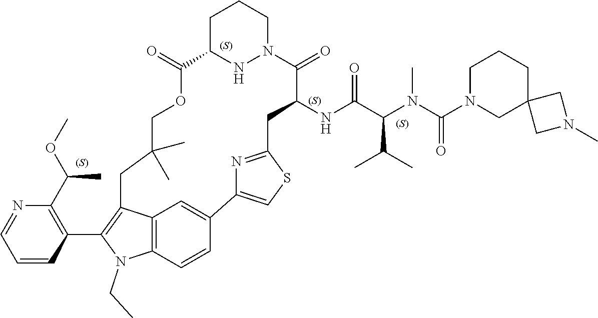 |
| AA590 | 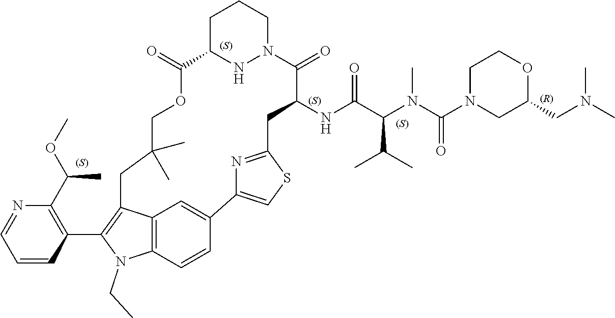 |
| AA591 | 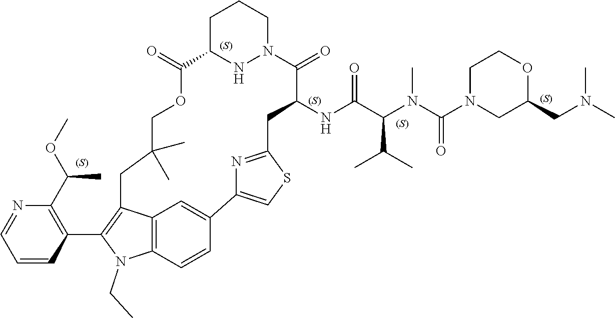 |
| AA592 | 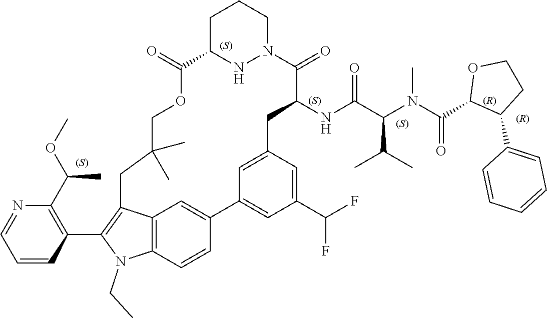 |
| AA593 | 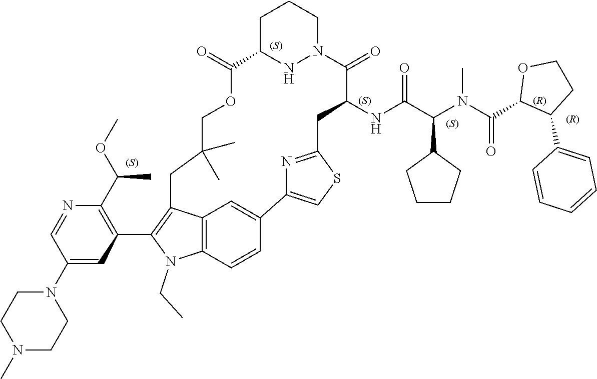 |
| AA594 | 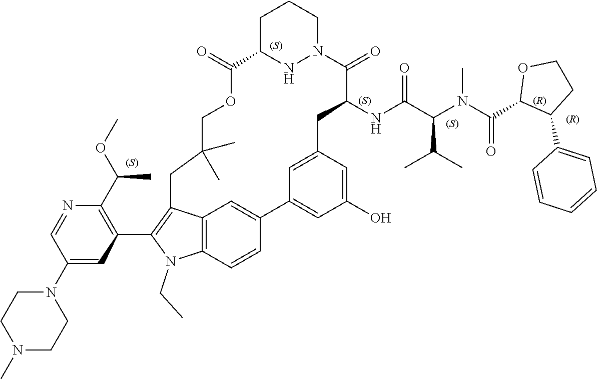 |
| AA595 | 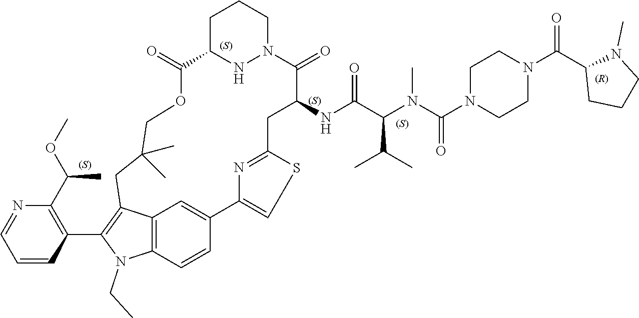 |
| AA596 | 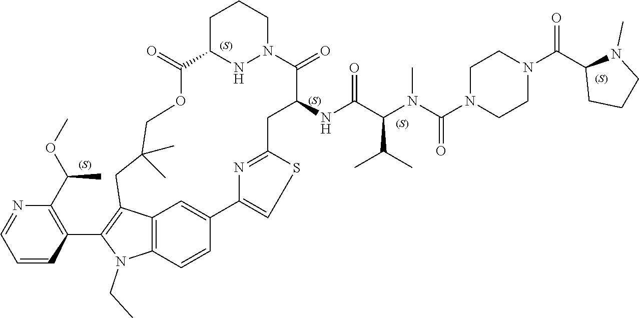 |
| AA597 | 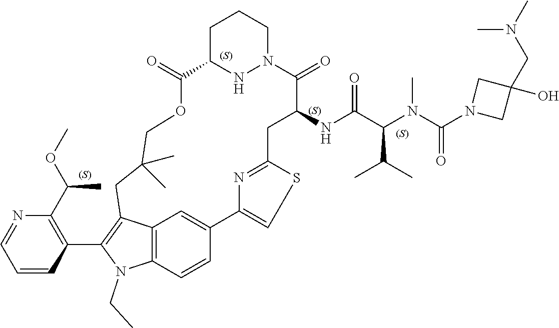 |
| AA598 | 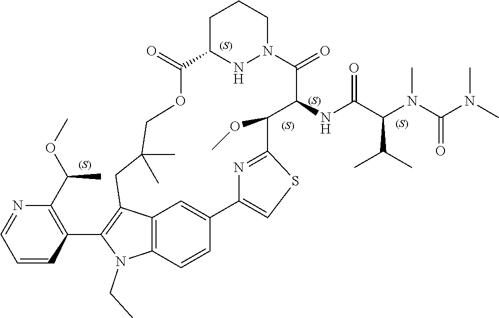 |
| AA599 | 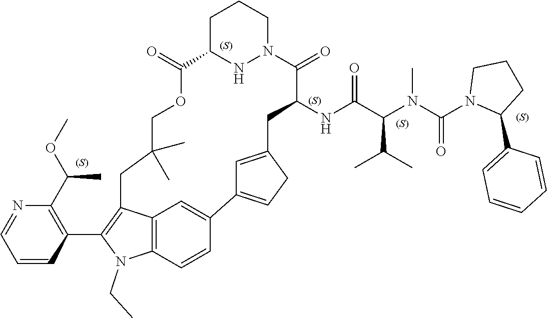 |
| AA600 | 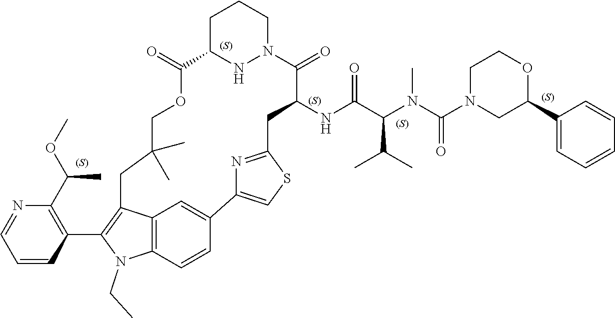 |
| AA601 | 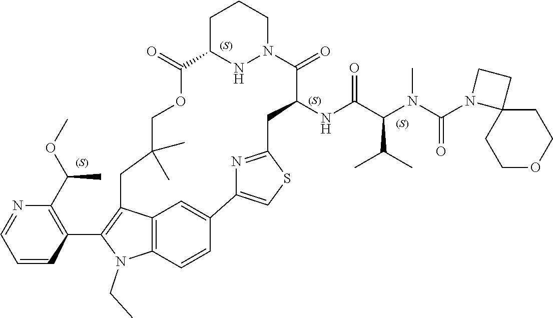 |
| AA602 | 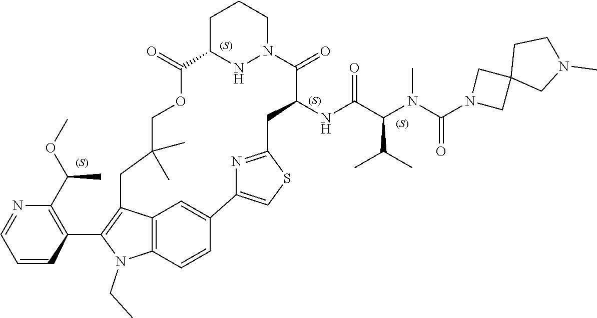 |
| AA603 | 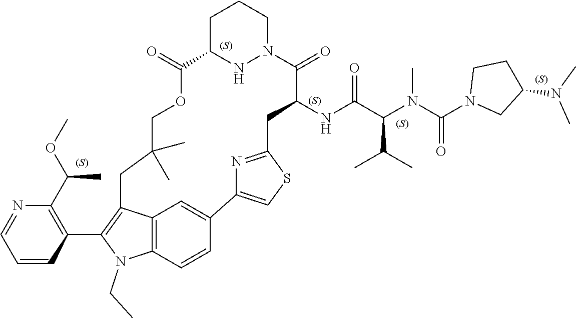 |
| AA604 | 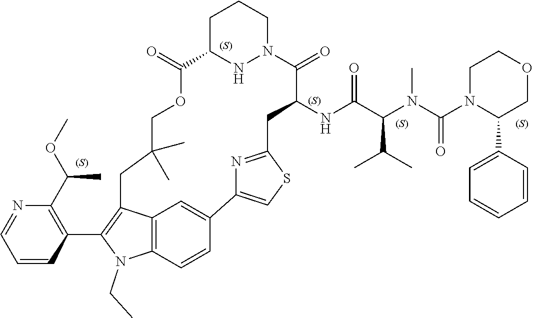 |
| AA605 | 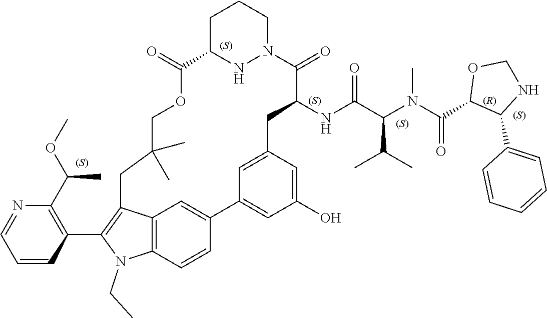 |
| AA606 | 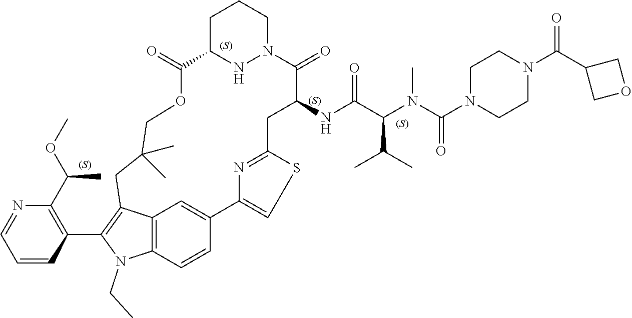 |
| AA607 | 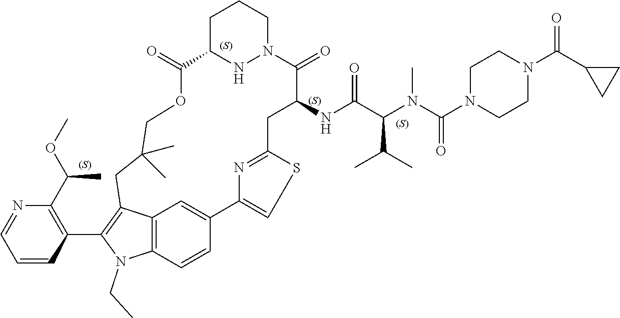 |
| AA608 | 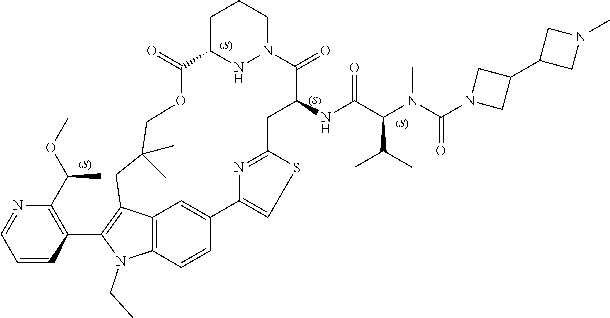 |
| AA609 | 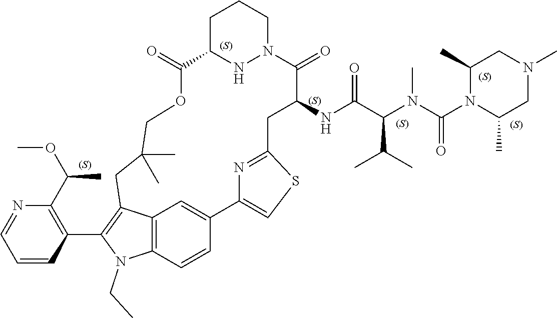 |
| AA610 | 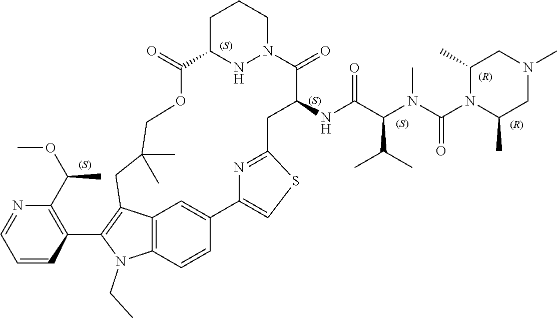 |
| AA611 | 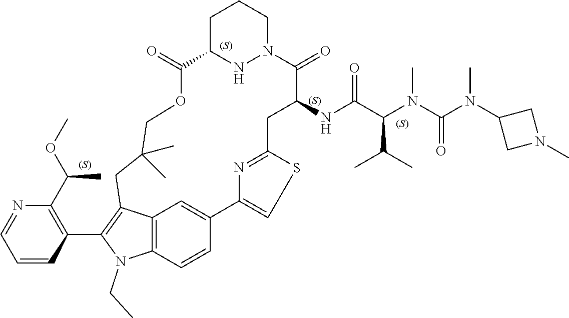 |
| AA612 | 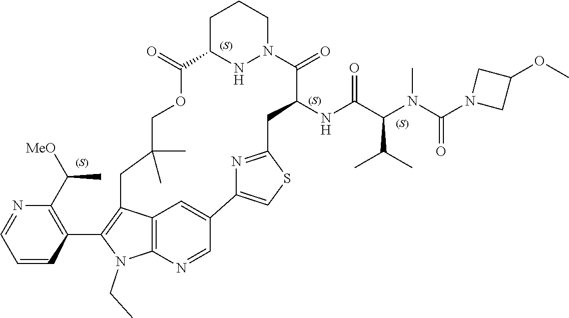 |
| AA613 | 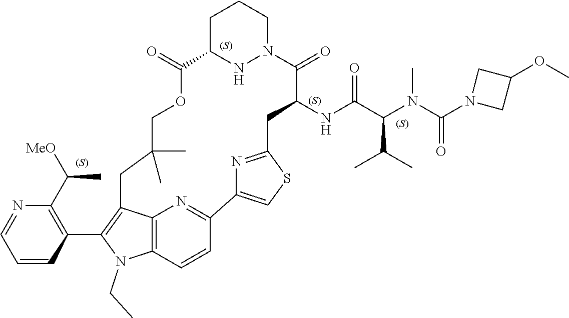 |
| AA614 | 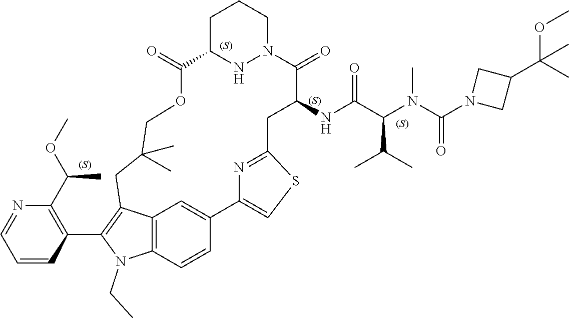 |

Note
that some compounds are shown with bonds as flat or wedged. In some instances, the relative stereochemistry of stereoisomers has
been determined; in some instances, the absolute stereochemistry has been determined. In some instances, a single Example number
corresponds to a mixture of stereoisomers. All stereoisomers of the compounds of the foregoing table are contemplated by the present
invention. In particular embodiments, an atropisomer of a compound of the foregoing table is contemplated. Any compound shown in
brackets indicates that the compound is a diastereomer, and the absolute stereochemistry of such diastereomer may not be known.

In some embodiments, a compound of Table A2 is provided, or a pharmaceutically acceptable salt thereof. In some embodiments, the RAS(ON) inhibitor is selected from Table A2, or a pharmaceutically acceptable salt or atropisomer thereof.

TABLE A2
Certain Compounds of the Present Invention

| Ex# | Structure |
|---|---|
| AB 4 | 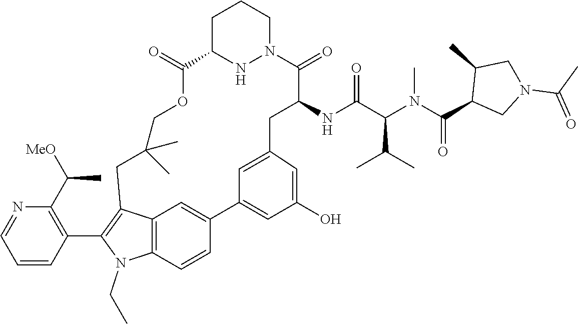 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| AB 5 | 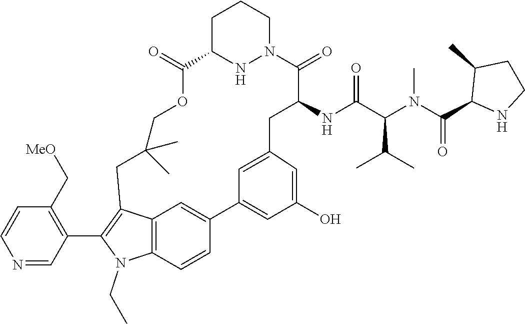 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| AB 6 | 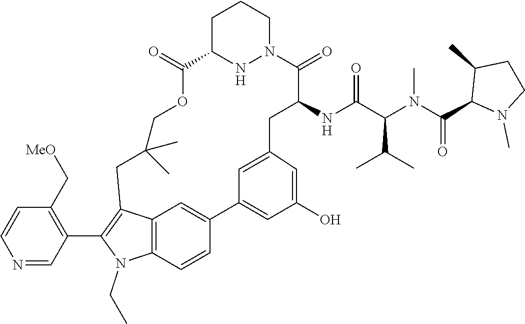 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| AB 8 | 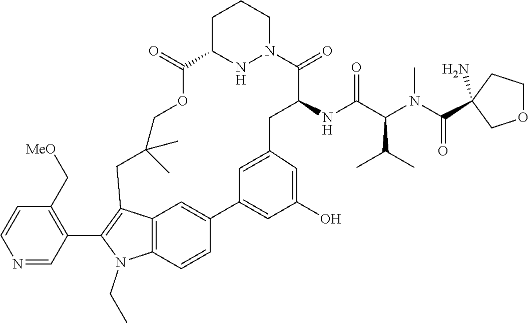 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| AB 9 | 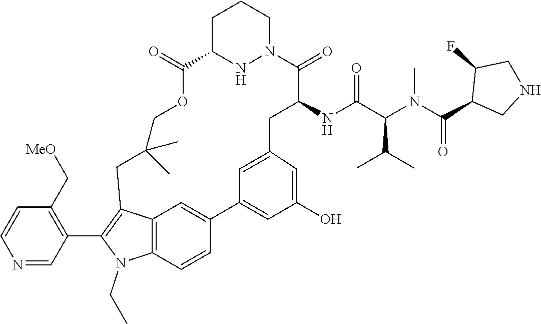 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| AB 12 | 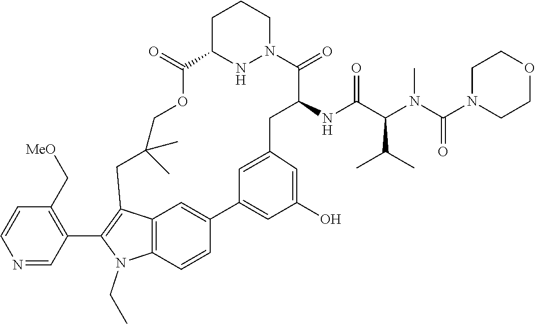 [SMILES: CCn1c(-c2cnccc2COC)c2c3cc(ccc31)-c1cc(O)cc(c1)C[C@H](NC(=O)[C@H](C(C)C)N(C)C(=O)N1CCOCC1)C(=O)N1CCC[C@H](N1)C(=O)OCC(C)(C)C2] |
| AB 13 | 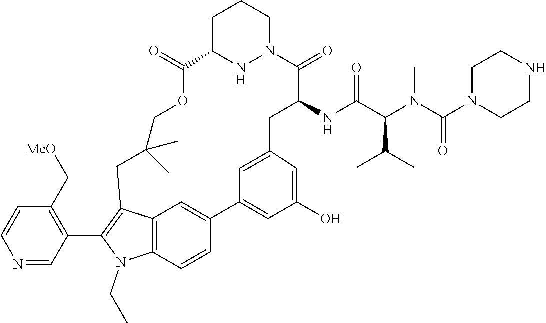 [SMILES: CCn1c(-c2cnccc2COC)c2c3cc(ccc31)-c1cc(O)cc(c1)C[C@H](NC(=O)[C@H](C(C)C)N(C)C(=O)N1CCNCC1)C(=O)N1CCC[C@H](N1)C(=O)OCC(C)(C)C2] |
| AB 19 | 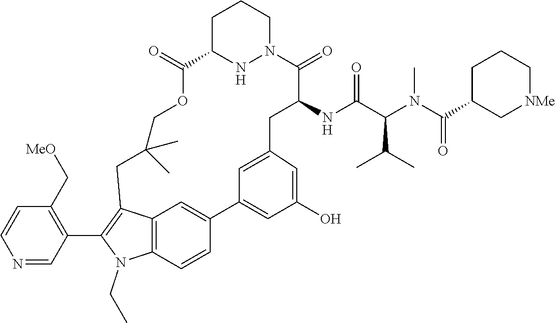 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCN(C)C1)C(=O)N1CCC[C@H](N1)C(=O)OCC(C)(C)C2] |
| AB 44 | 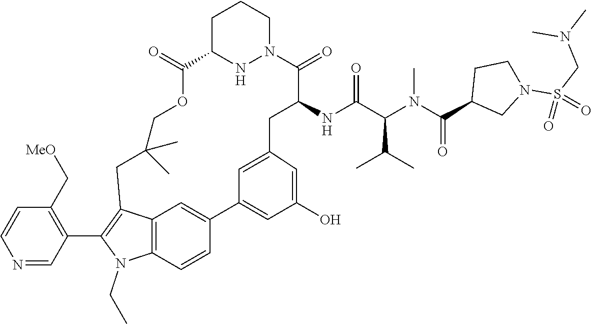 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(S(=O)(=O)CN(C)C)C1)C(=O)N1CCC[C@H](N1)C(=O)OCC(C)(C)C2] |
| AB 47 | 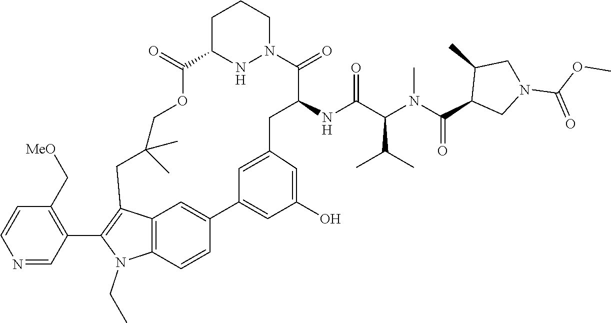 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CN(C(=O)OC)C[C@@H]1C)C(=O)N1CCC[C@H](N1)C(=O)OCC(C)(C)C2] |
| AB 57 | 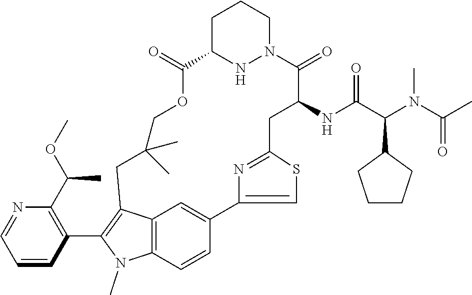 |
| AB 58 | 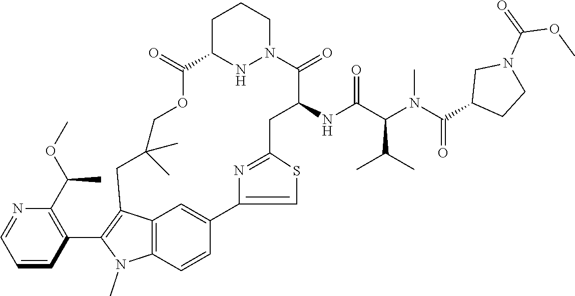 |
| AB 59 | 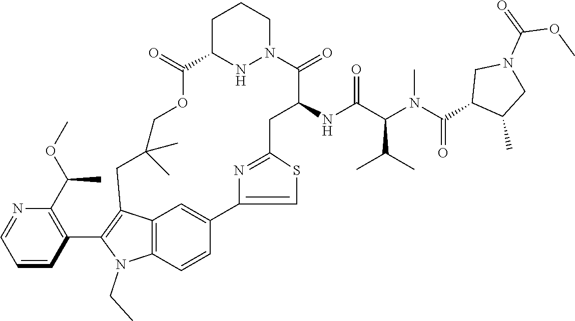 |
| AB 60 | 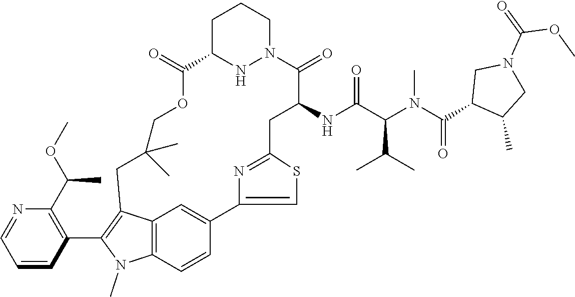 |
| AB 61 | 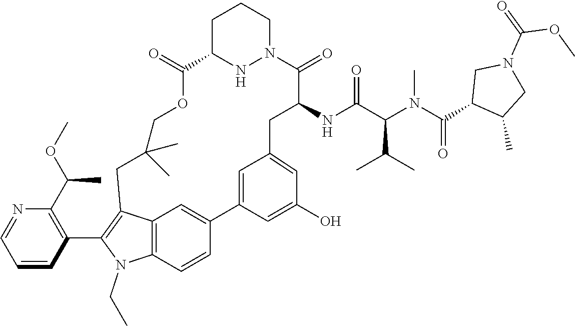 |
| AB 66 | 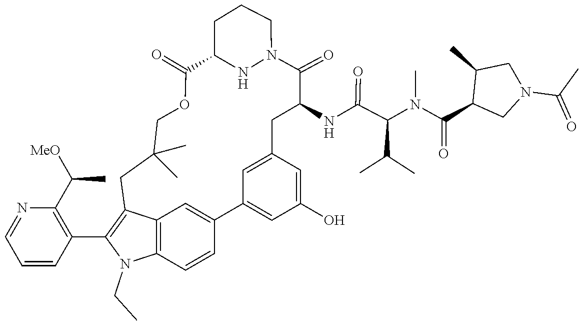 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CN(C(C)=O)C[C@@H]1C)C(=O)N1CCC[C@H](N1)C(=O)OCC(C)(C)C2] |
| AB 67 | 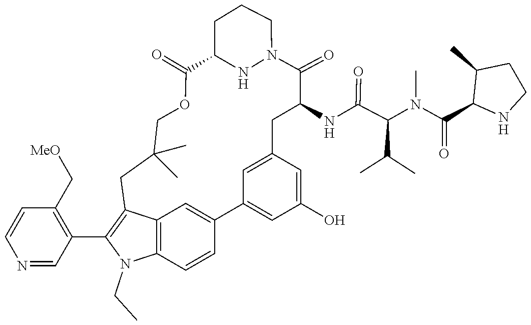 [SMILES: CCn1c(-c2cnccc2COC)c2c3cc(ccc31)-c1cc(O)cc(c1)C[C@H](NC(=O)[C@H](C(C)C)N(C)C(=O)[C@@H]1NCC[C@@H]1C)C(=O)N1CCC[C@H](N1)C(=O)OCC(C)(C)C2] |
| AB 69 | 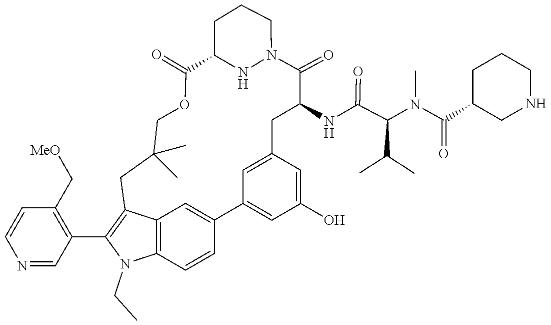 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CCCNC1)C(=O)N1CCC[C@H](N1)C(=O)OCC(C)(C)C2] |
| AB 71 | 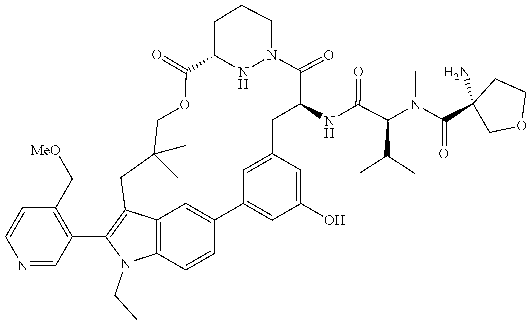 [SMILES: CCn1c(-c2cnccc2COC)c2c3cc(ccc31)-c1cc(O)cc(c1)C[C@H](NC(=O)[C@H](C(C)C)N(C)C(=O)[C@@]1(N)CCOC1)C(=O)N1CCC[C@H](N1)C(=O)OCC(C)(C)C2] |
| AB 73 | 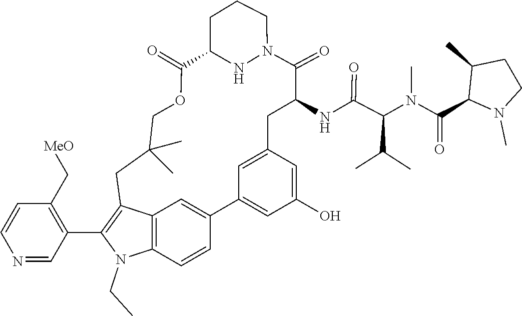 [SMILES: CCn1c(-c2cnccc2COC)c2c3cc(ccc31)-c1cc(O)cc(c1)C[C@H](NC(=O)[C@H](C(C)C)N(C)C(=O)[C@H]1[C@@H](C)CCN1C)C(=O)N1CCC[C@H](N1)C(=O)OCC(C)(C)C2] |
| AB 74 | 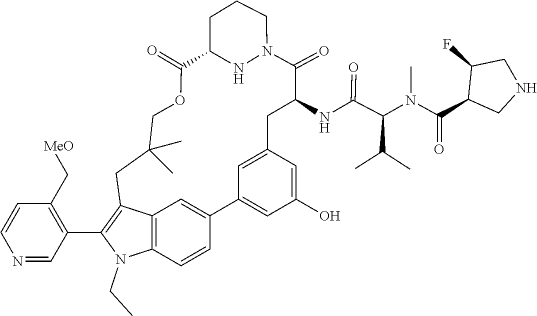 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1F)C(=O)N1CCC[C@H](N1)C(=O)OCC(C)(C)C2] |
| AB 80 | 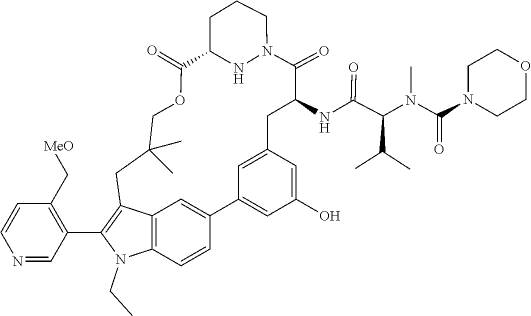 |
| AB 81 | 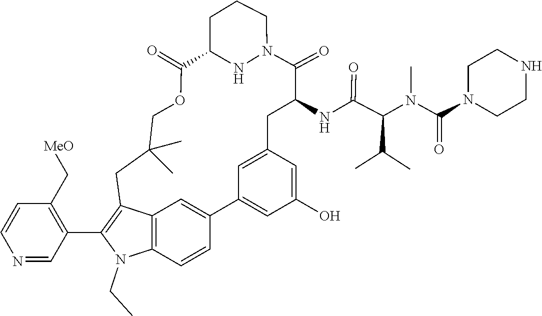 |
| AB 94 | 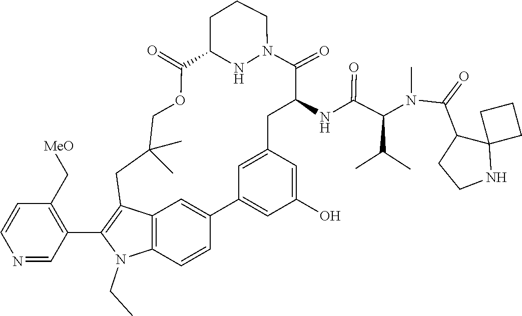 [SMILES: CCn1c(-c2cnccc2COC)c2c3cc(ccc31)-c1cc(O)cc(c1)C[C@H](NC(=O)[C@H](C(C)C)N(C)C(=O)C1CCNC13CCC3)C(=O)N1CCC[C@H](N1)C(=O)OCC(C)(C)C2] |
| AB 95 | 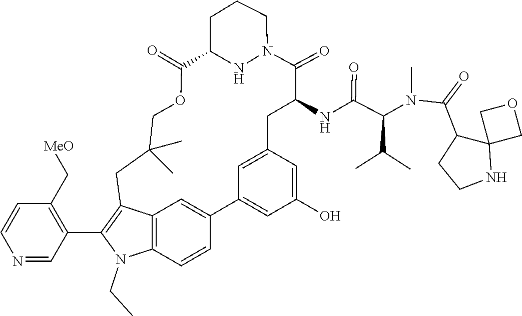 [SMILES: CCn1c(-c2cnccc2COC)c2c3cc(ccc31)-c1cc(O)cc(c1)C[C@H](NC(=O)[C@H](C(C)C)N(C)C(=O)C1CCNC13COC3)C(=O)N1CCC[C@H](N1)C(=O)OCC(C)(C)C2] |
| AB 96 | 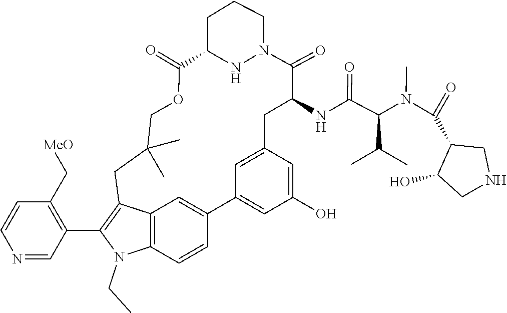 [SMILES: CCn1c(-c2cnccc2COC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| AB 97 | 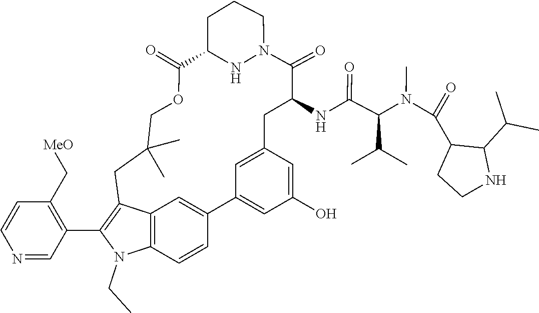 [SMILES: CCn1c(-c2cnccc2COC)c2c3cc(ccc31)-c1cc(O)cc(c1)C[C@H](NC(=O)[C@H](C(C)C)N(C)C(=O)C1CCNC1C(C)C)C(=O)N1CCC[C@H](N1)C(=O)OCC(C)(C)C2] |
| AB 99 | 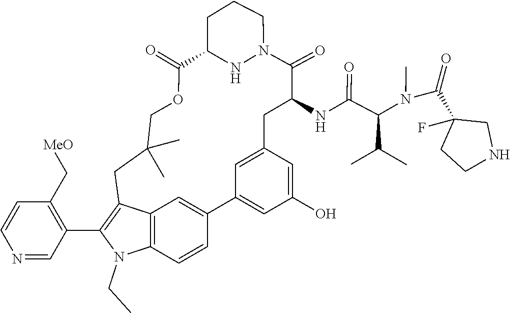 [SMILES: CCn1c(-c2cnccc2COC)c2c3cc(ccc31)-c1cc(O)cc(c1)C[C@H](NC(=O)[C@H](C(C)C)N(C)C(=O)[C@@]1(F)CCNC1)C(=O)N1CCC[C@H](N1)C(=O)OCC(C)(C)C2] |
| AB 100 | 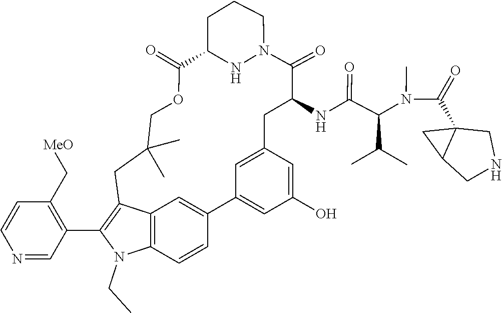 [SMILES: CCn1c(-c2cnccc2COC)c2c3cc(ccc31)-c1cc(O)cc(c1)C[C@H](NC(=O)[C@H](C(C)C)N(C)C(=O)[C@]13CNCC1C3)C(=O)N1CCC[C@H](N1)C(=O)OCC(C)(C)C2] |
| AB 104 | 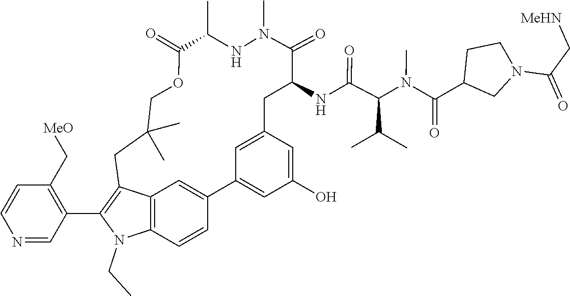 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)CNC)C1)C(=O)N(C)N[C@@H](C)C(=O)OCC(C)(C)C2] |
| AB 106 | 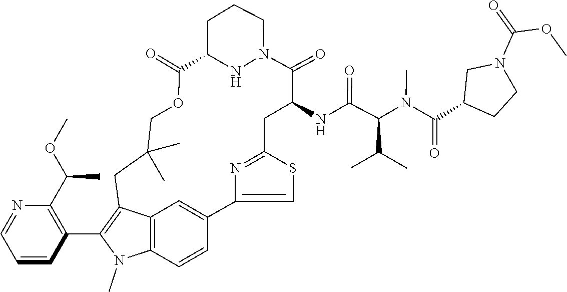 |
| AB 107 | 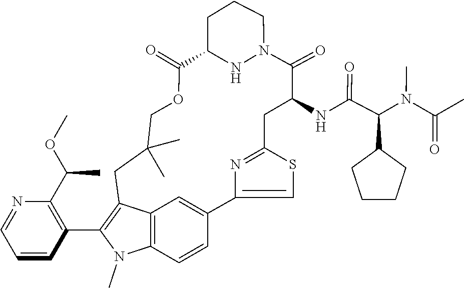 |
| AB 109 | 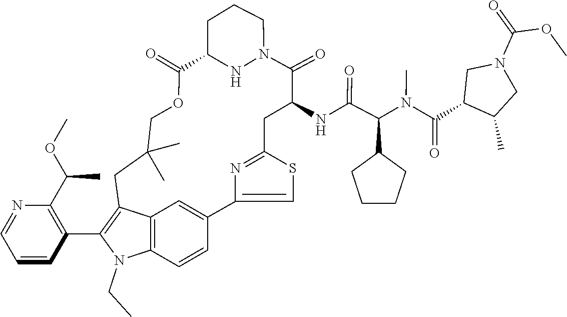 |
| AB 110 | 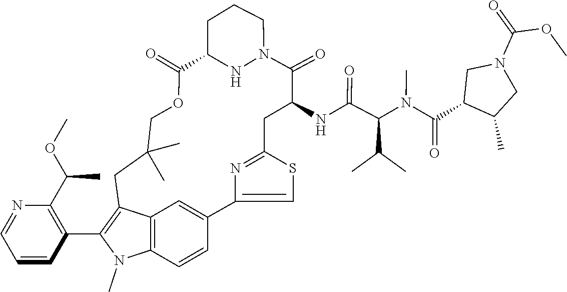 |
| AB 111 | 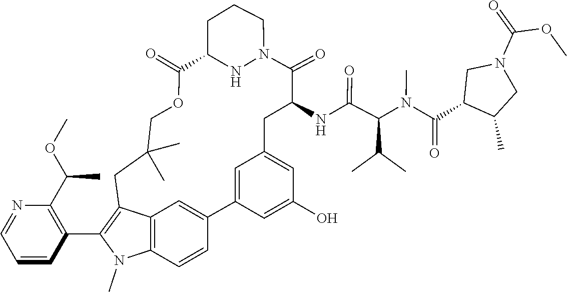 |
| AB 112 | 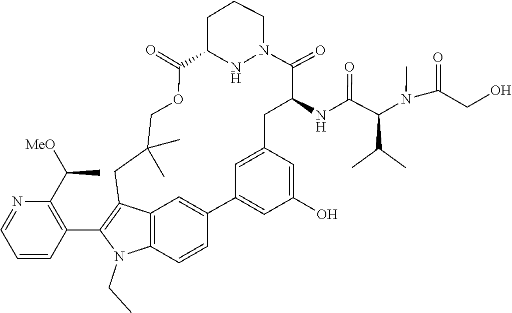 [SMILES: CCn1c(-c2cccnc2[C@H](C)OC)c2c3cc(ccc31)-c1cc(O)cc(c1)C[C@H](NC(=O)[C@H](C(C)C)N(C)C(=O)CO)C(=O)N1CCC[C@H](N1)C(=O)OCC(C)(C)C2] |
| AB 113 | 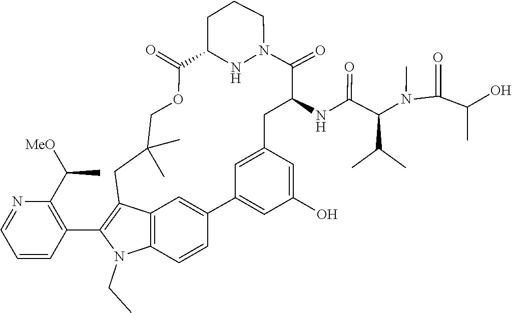 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(C)O)C(=O)N1CCC[C@H](N1)C(=O)OCC(C)(C)C2] |
| AB 114 | 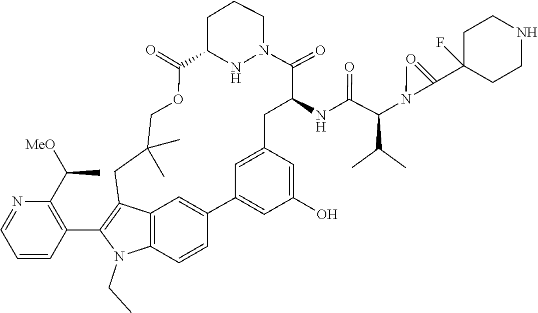 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(F)CCNCC1)C(=O)N1CCC[C@H](N1)C(=O)OCC(C)(C)C2] |
| AB 117 | 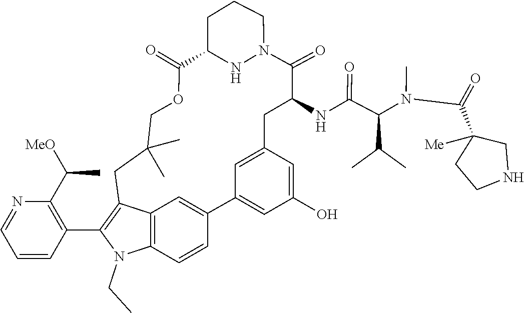 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@]1(C)CCNC1)C(=O)N1CCC[C@H](N1)C(=O)OCC(C)(C)C2] |
| AB 119 | 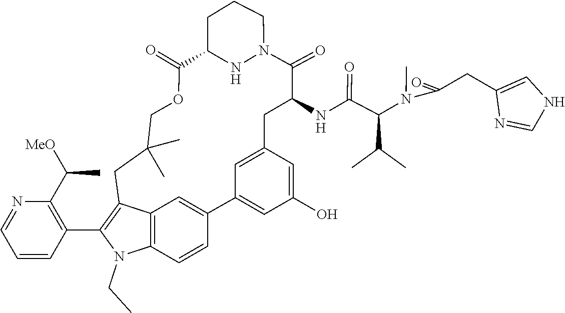 [SMILES: CCn1c(-c2cccnc2[C@H](C)OC)c2c3cc(ccc31)-c1cc(O)cc(c1)C[C@H](NC(=O)[C@H](C(C)C)N(C)C(=O)Cc1c[nH]cn1)C(=O)N1CCC[C@H](N1)C(=O)OCC(C)(C)C2] |
| AB 122 | 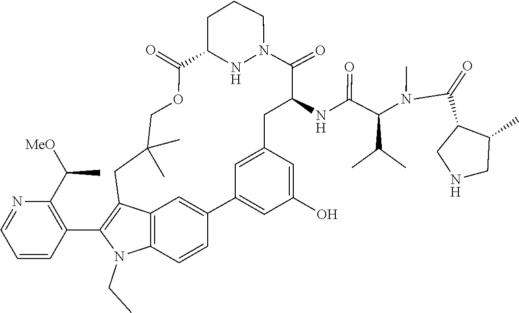 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CNC[C@H]1C)C(=O)N1CCC[C@H](N1)C(=O)OCC(C)(C)C2] |
| AB 123 | 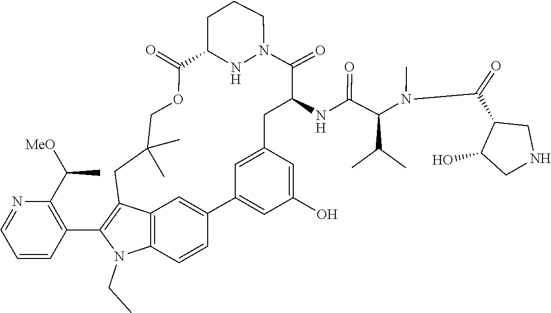 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@@H]1CNC[C@@H]1O)C(=O)N1CCC[C@H](N1)C(=O)OCC(C)(C)C2] |
| AB 124 | 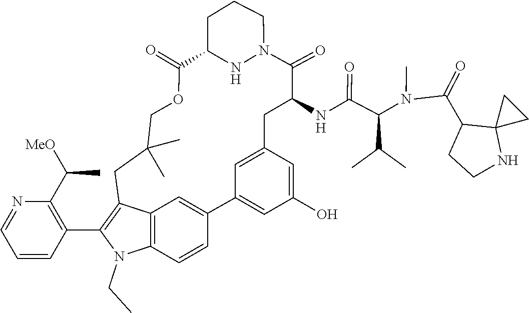 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCNC13CC3)C(=O)N1CCC[C@H](N1)C(=O)OCC(C)(C)C2] |
| AB 126 | 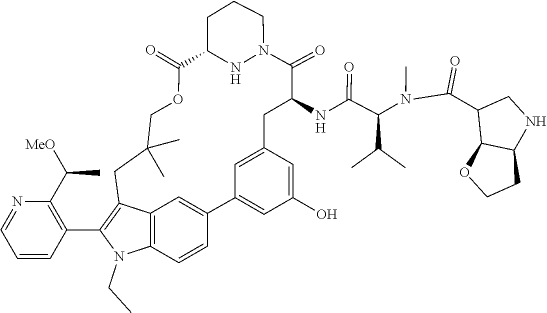 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CN[C@H]3CCO[C@@H]13)C(=O)N1CCC[C@H](N1)C(=O)OCC(C)(C)C2] |
| AB 128 | 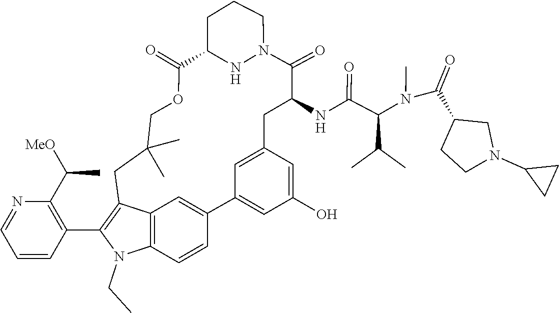 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C3CC3)C1)C(=O)N1CCC[C@H](N1)C(=O)OCC(C)(C)C2] |
| AB 129 | 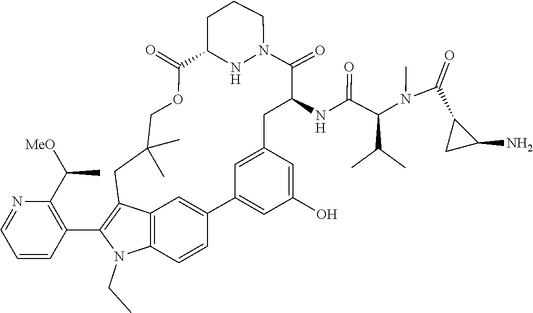 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@@H]1N)C(=O)N1CCC[C@H](N1)C(=O)OCC(C)(C)C2] |
| AB 130 | 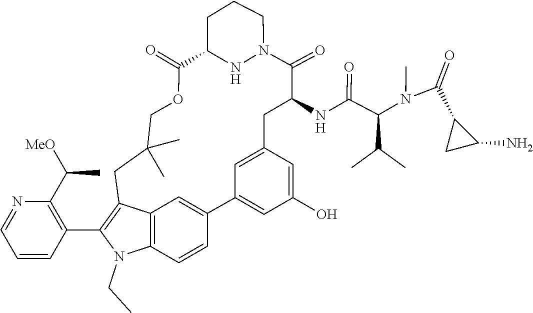 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1C[C@H]1N)C(=O)N1CCC[C@H](N1)C(=O)OCC(C)(C)C2] |
| AB 133 | 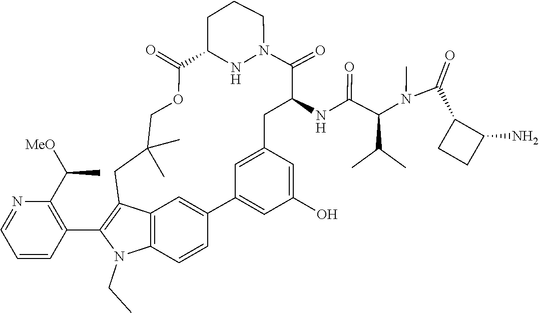 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CC[C@H]1N)C(=O)N1CCC[C@H](N1)C(=O)OCC(C)(C)C2] |
| AB 134 | 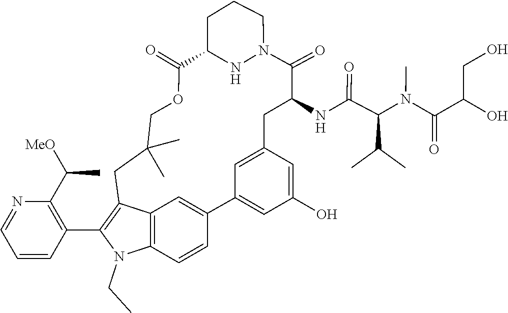 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C(O)CO)C(=O)N1CCC[C@H](N1)C(=O)OCC(C)(C)C2] |
| AB 135 | 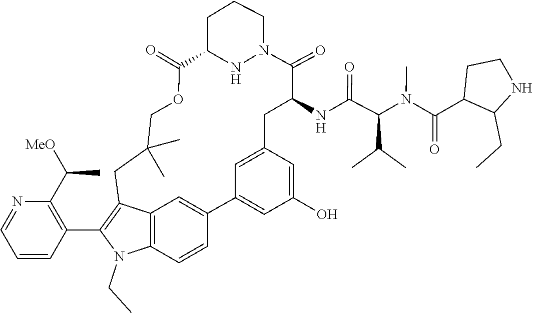 [SMILES: CCC1NCCC1C(=O)N(C)[C@H](C(=O)N[C@H]1Cc2cc(O)cc(c2)-c2ccc3c(c2)c(c(-c2cccnc2[C@H](C)OC)n3CC)CC(C)(C)COC(=O)[C@@H]2CCCN(N2)C1=O)C(C)C] |
| AB 137 | 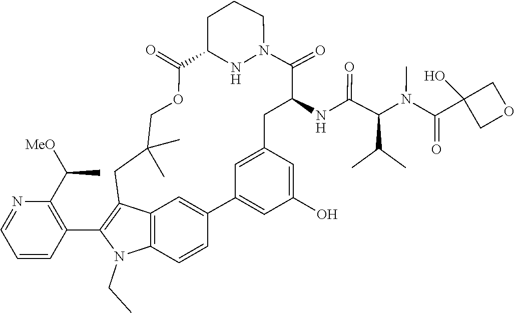 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1(O)COC1)C(=O)N1CCC[C@H](N1)C(=O)OCC(C)(C)C2] |
| AB 138 | 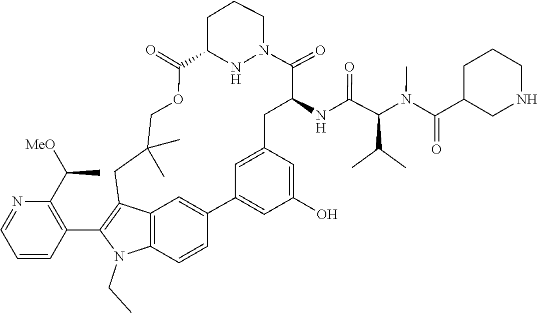 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CCCNC1)C(=O)N1CCC[C@H](N1)C(=O)OCC(C)(C)C2] |
| AB 139 | 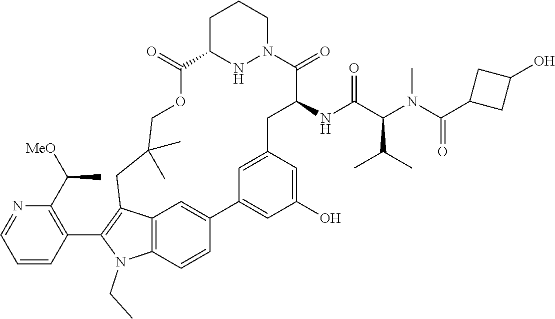 [SMILES: CCn1c(-c2cccnc2[C@H](C)OC)c2c3cc(ccc31)-c1cc(O)cc(c1)C[C@H](NC(=O)[C@H](C(C)C)N(C)C(=O)C1CC(O)C1)C(=O)N1CCC[C@H](N1)C(=O)OCC(C)(C)C2] |
| AB 141 | 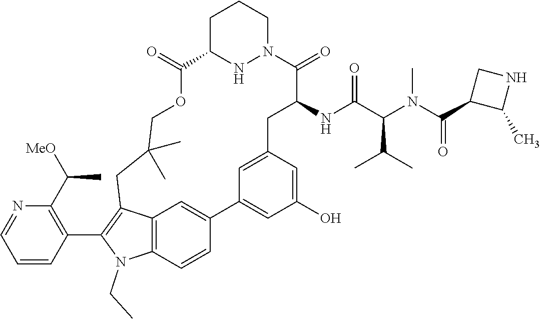 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CN[C@@H]1C)C(=O)N1CCC[C@H](N1)C(=O)OCC(C)(C)C2] |
| AB 143 | 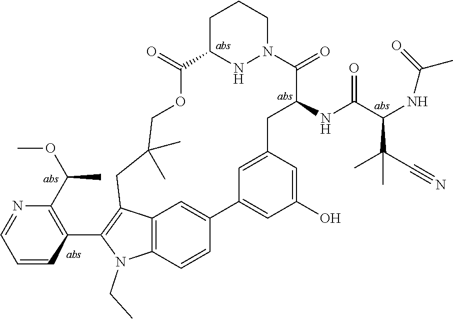 |
| AB 144 | 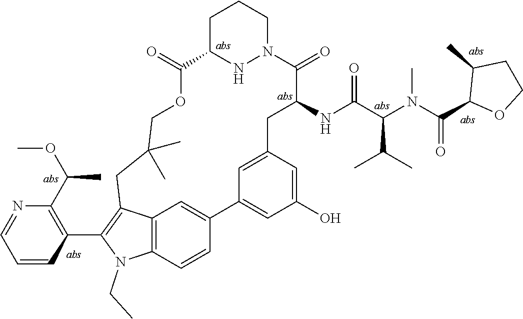 |
| AB 145 | 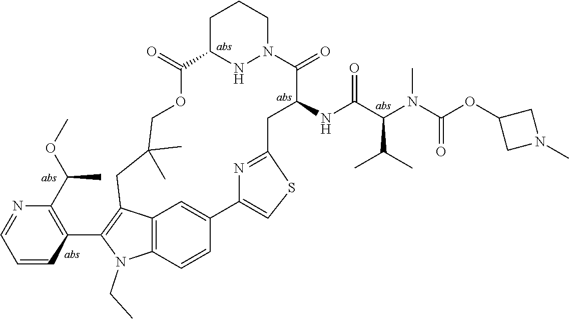 |
| AB 146 | 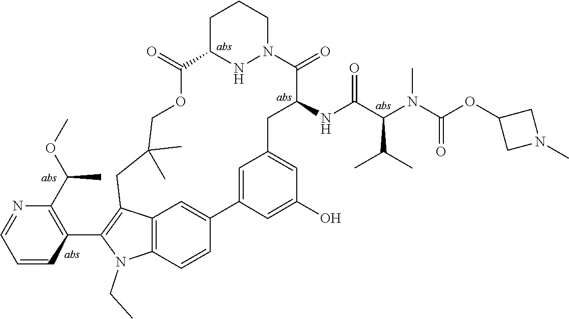 |
| AB 147 | 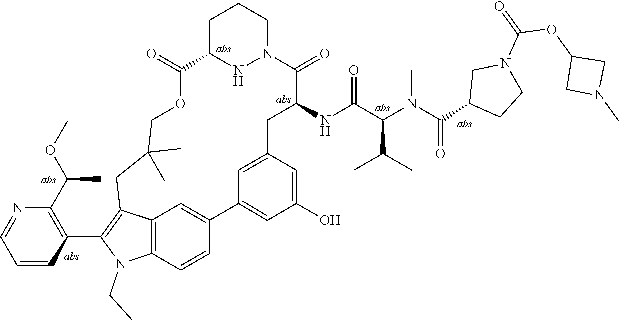 |
| AB 148 | 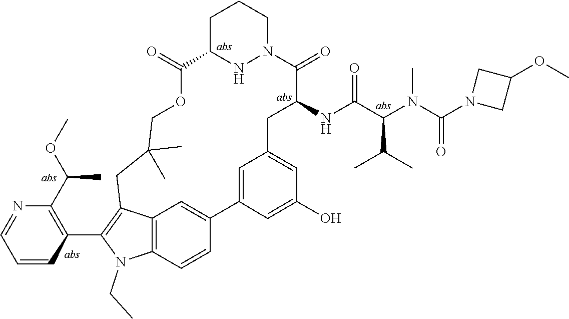 |
| AB 149 | 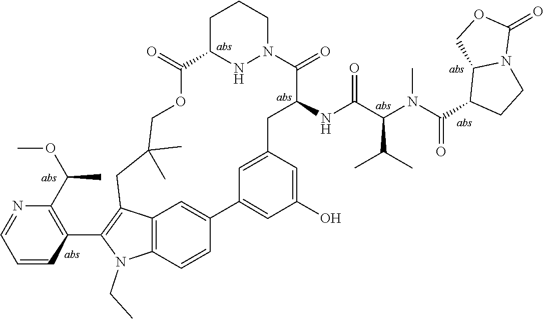 |
| AB 150 | 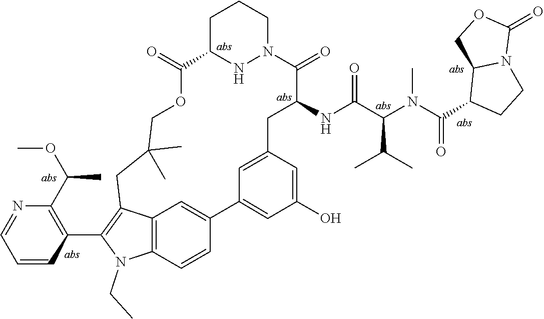 |
| AB 151 | 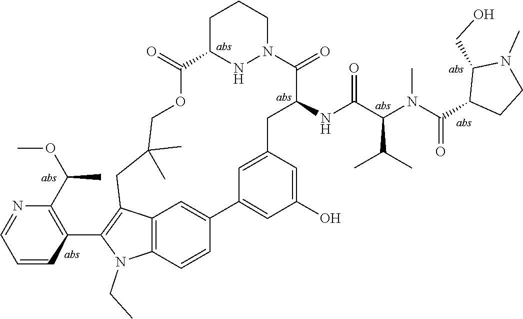 |
| AB 152 | 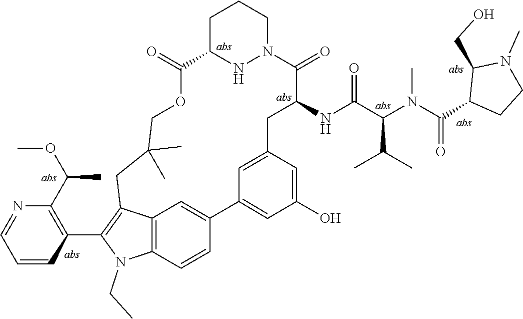 |
| AB 153 | 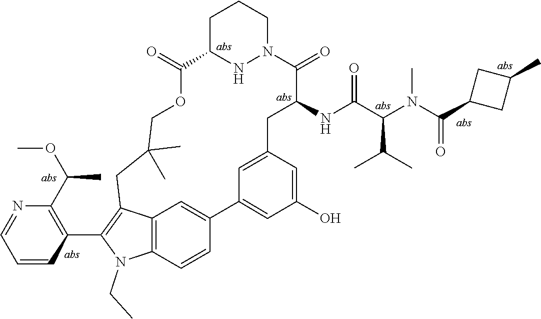 |
| AB 154 | 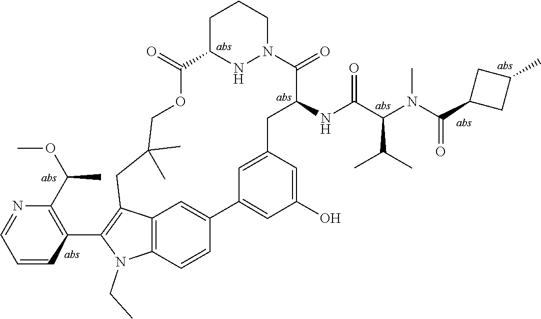 |
| AB 155 | 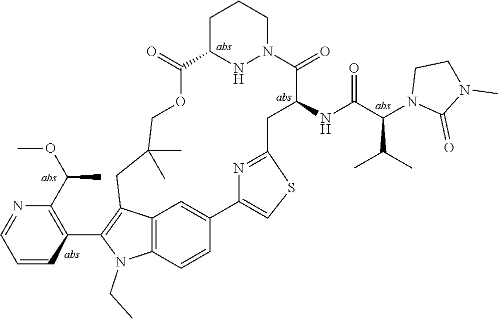 |
| AB 156 | 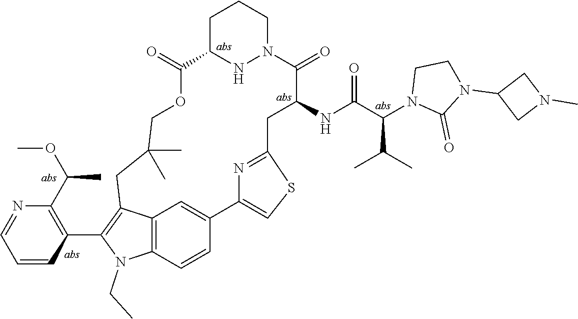 |
| AB 157 | 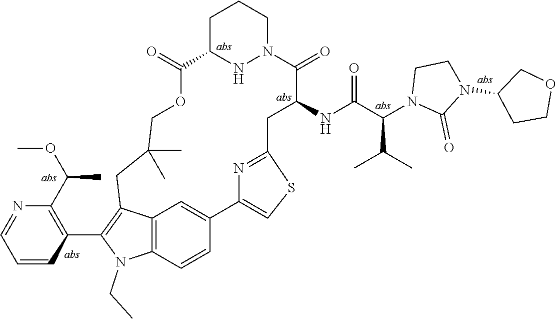 |
| AB 158 | 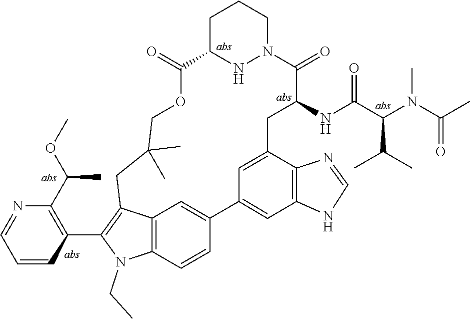 |
| AB 159 | 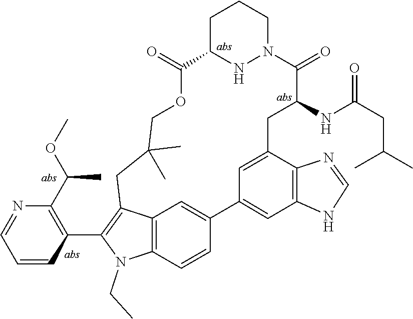 |
| AB 160 | 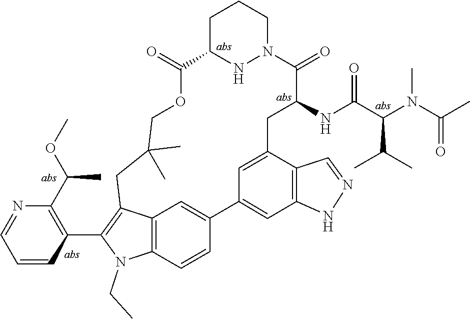 |
| AB 161 | 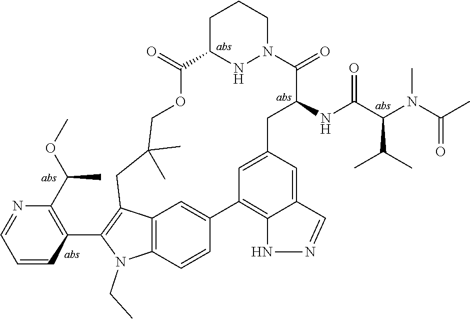 |
| AB 162 | 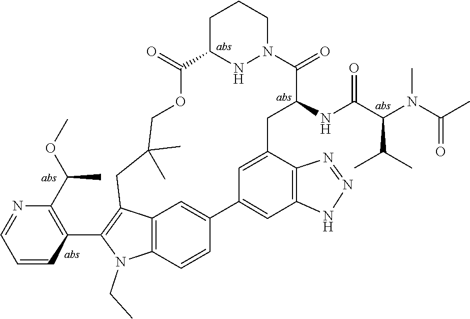 |
| AB 163 | 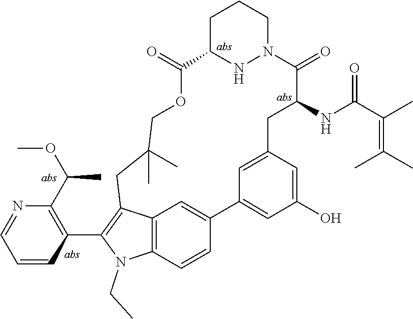 |
| AB 164 | 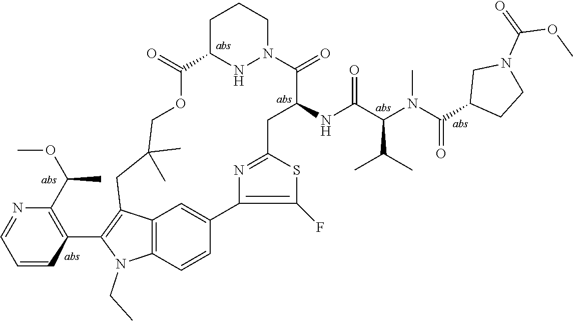 |
| AB 165 | 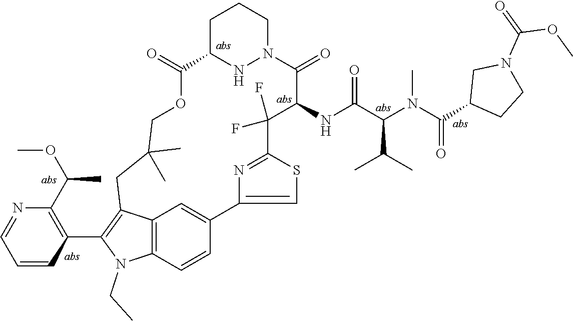 |
| AB 166 | 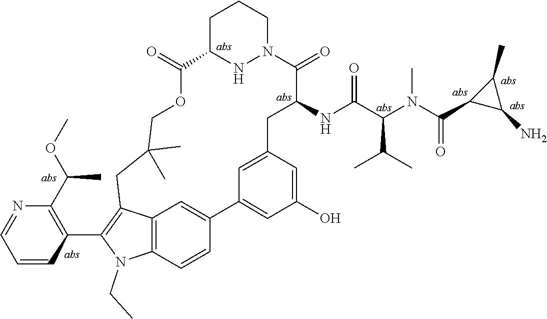 |
| AB 167 | 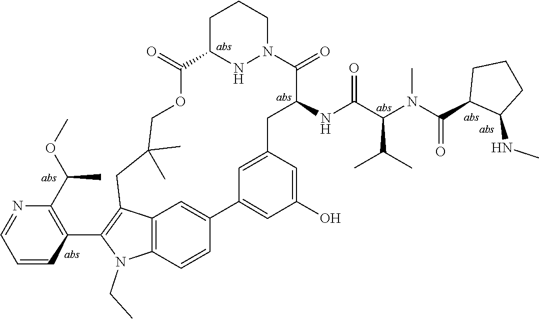 |
| AB 168 | 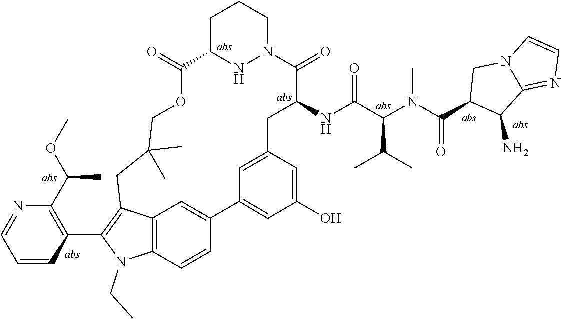 |
| AB 169 | 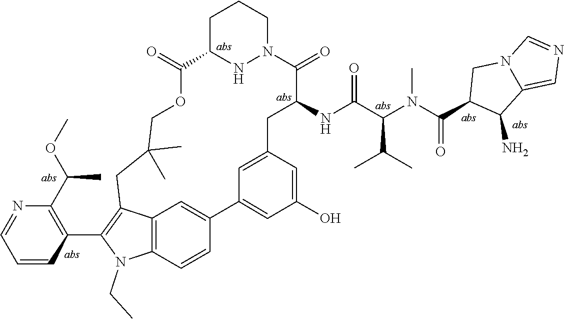 |
| AB 170 | 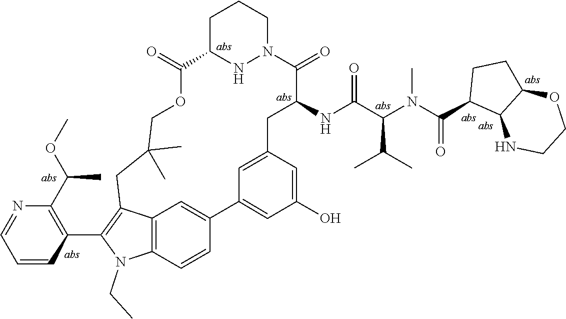 |
| AB 171 | 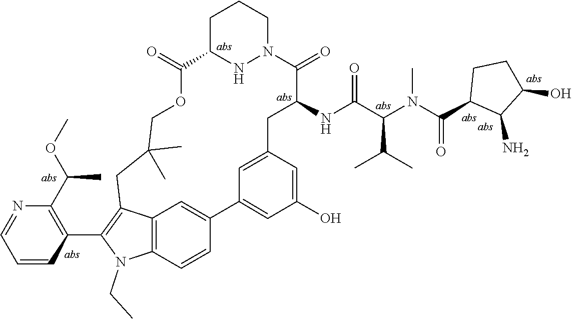 |
| AB 172 | 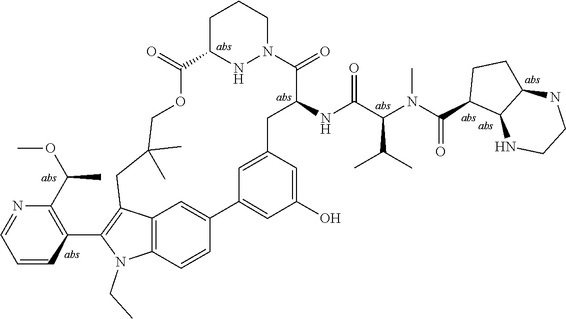 |
| AB 173 | 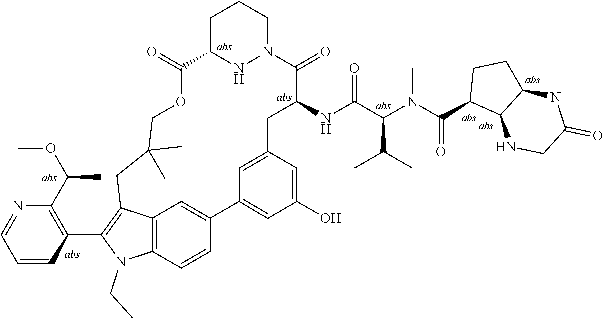 |
| AB 174 | 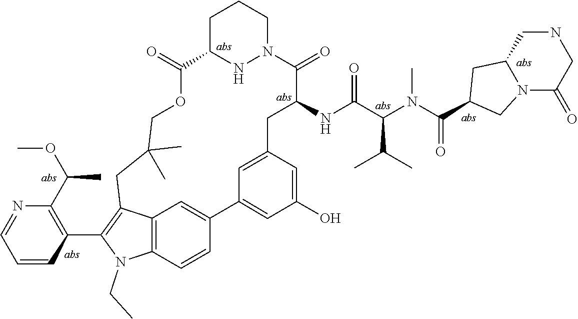 |
| AB 175 | 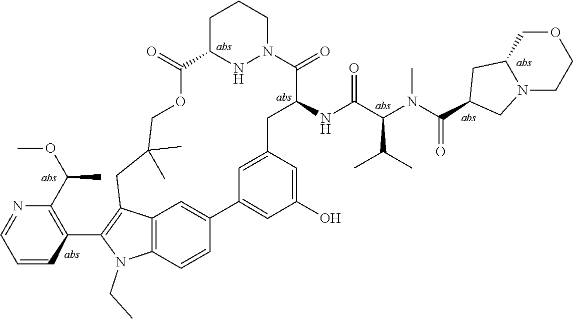 |
| AB 176 | 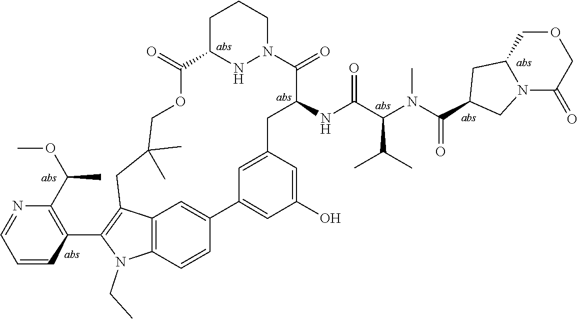 |
| AB 177 | 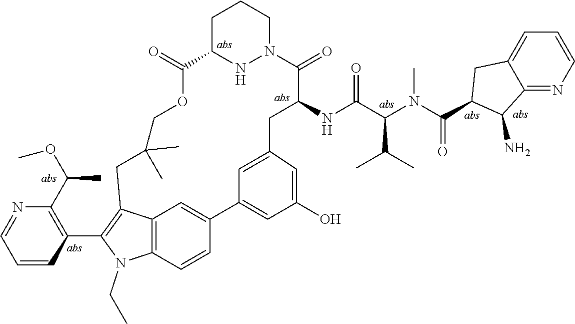 |
| AB 178 | 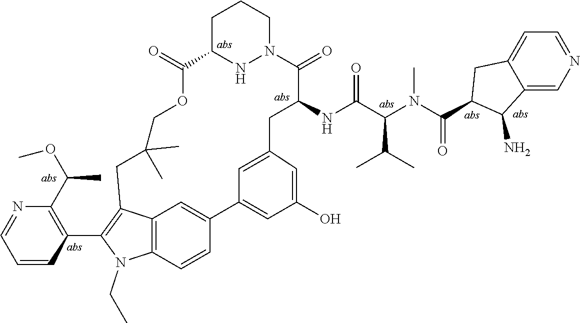 |
| AB 179 | 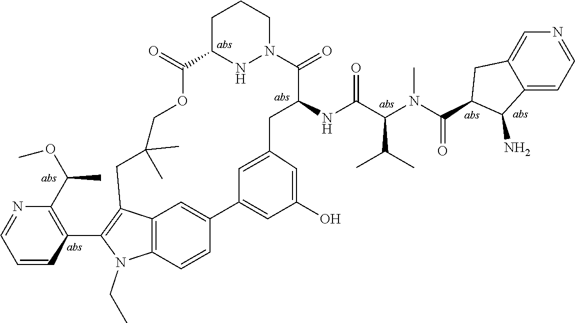 |
| AB 180 | 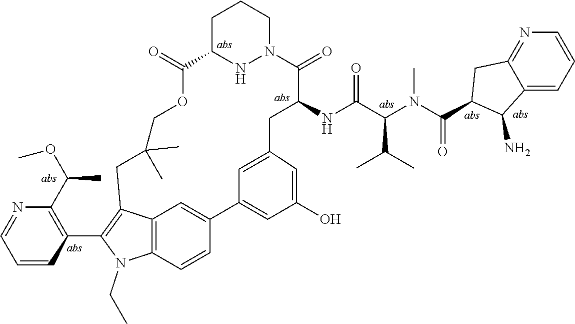 |
| AB 181 | 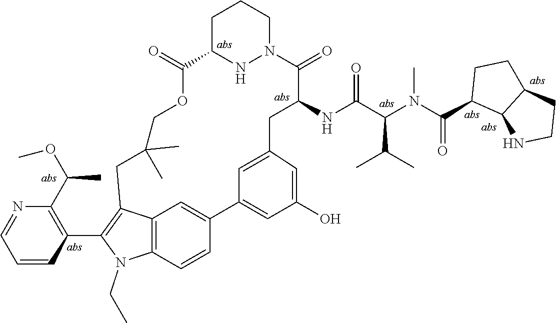 |
| AB 182 | 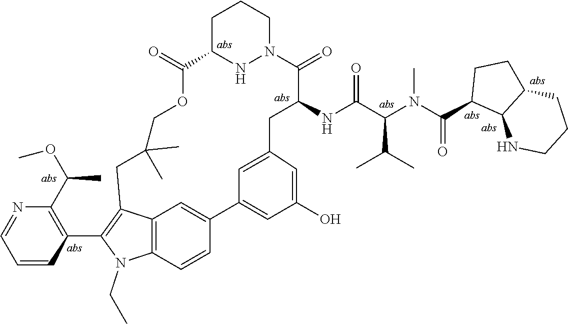 |
| AB 183 | 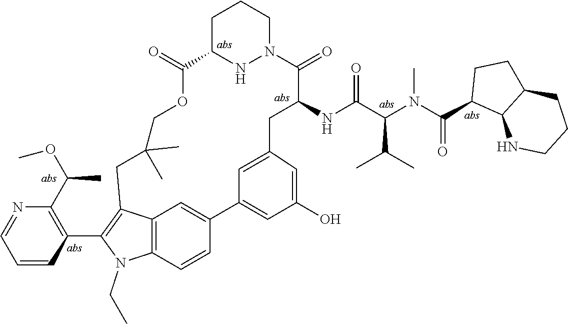 |
| AB 184 | 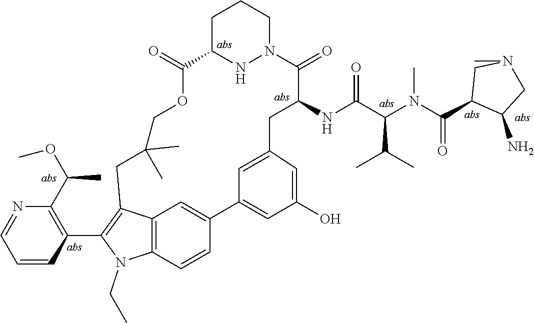 |
| AB 185 | 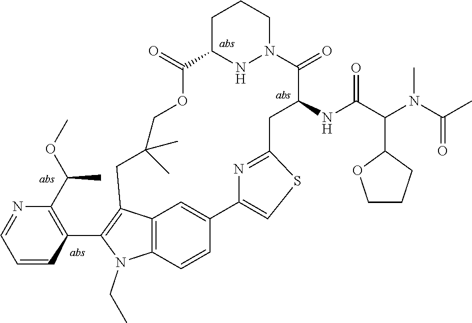 |
| AB 186 | 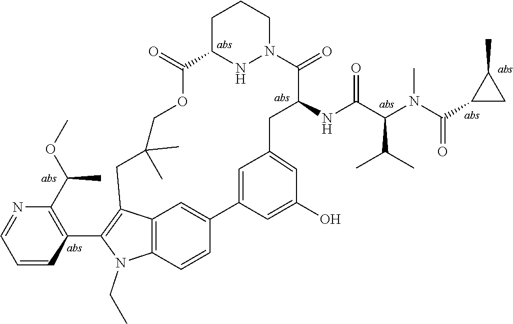 |
| AB 187 | 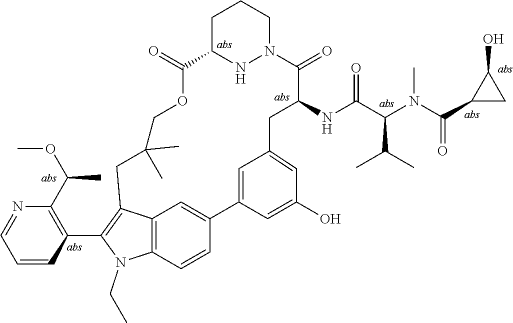 |
| AB 188 | 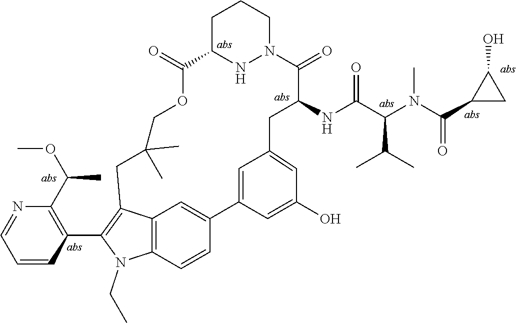 |
| AB 189 | 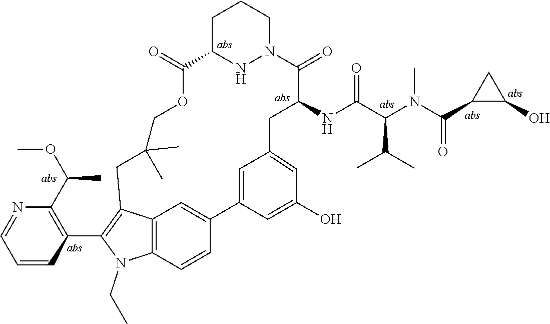 |
| AB 190 | 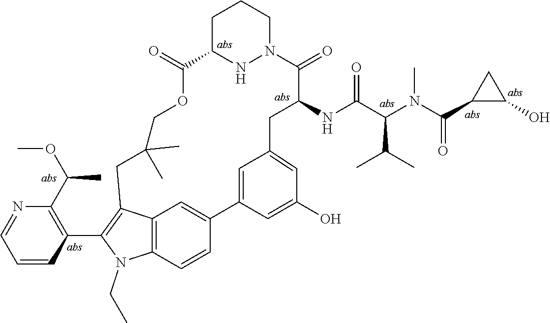 |
| AB 191 | 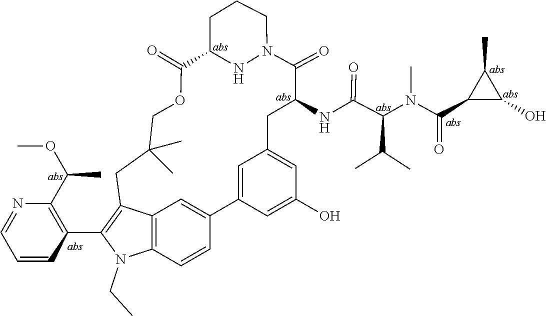 |
| AB 192 | 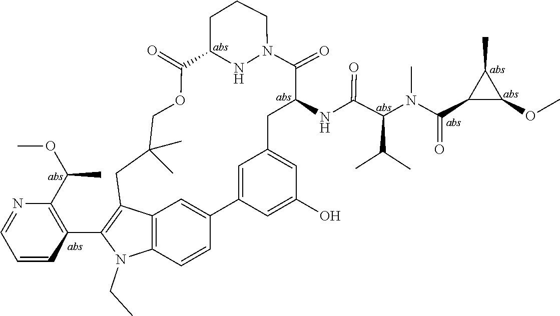 |
| AB 193 | 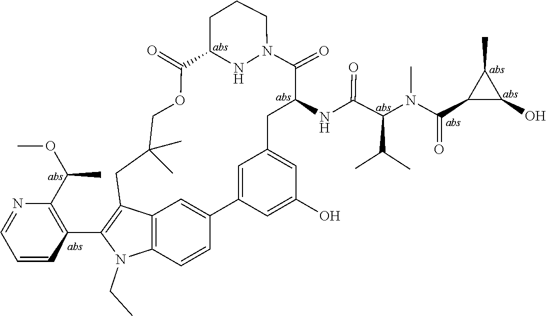 |
| AB 194 | 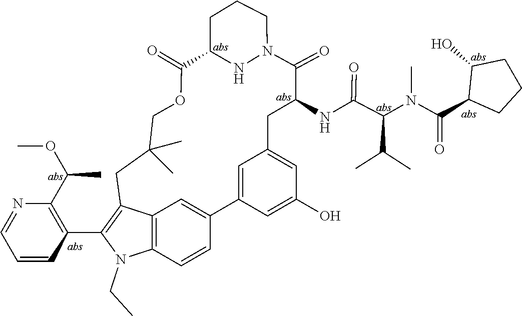 |
| AB 195 | 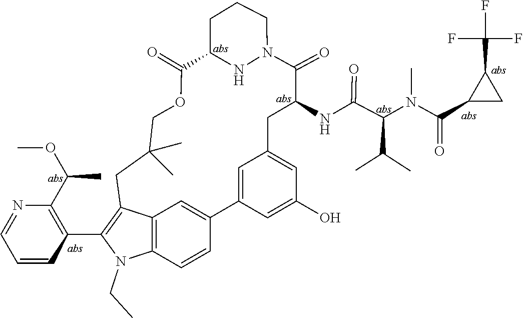 |
| AB 196 | 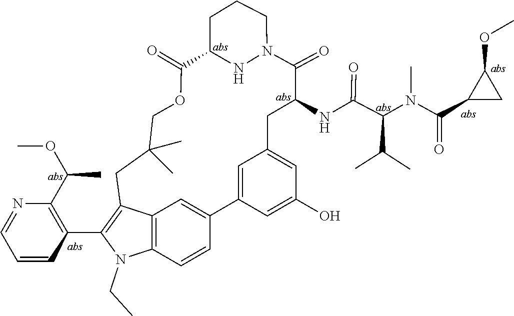 |
| AB 197 | 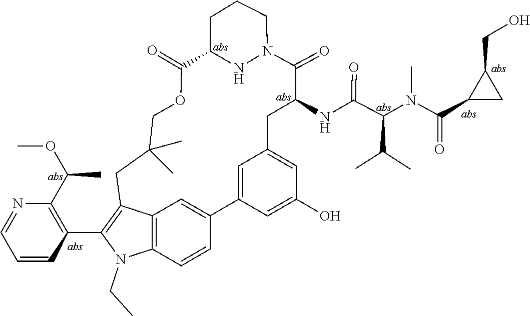 |
| AB 198 | 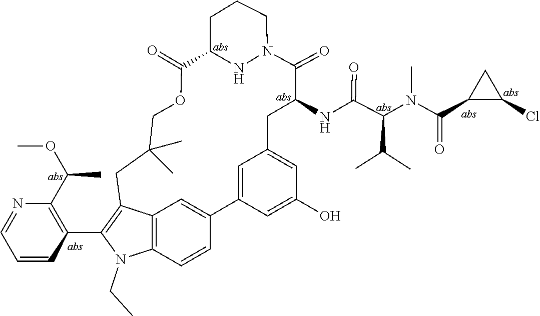 |
| AB 199 | 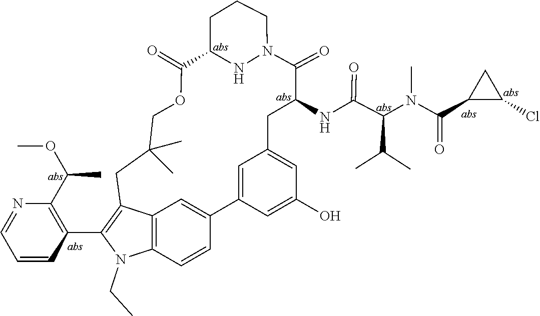 |
| AB 200 | 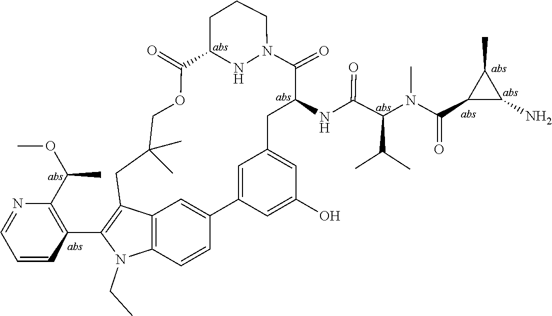 |
| AB 201 | 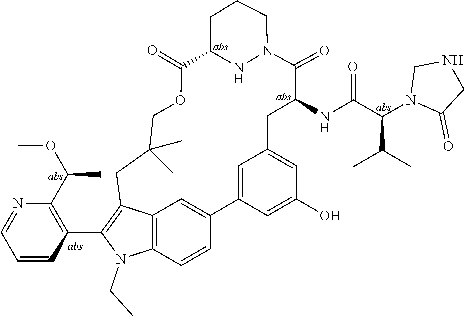 |
| AB 202 | 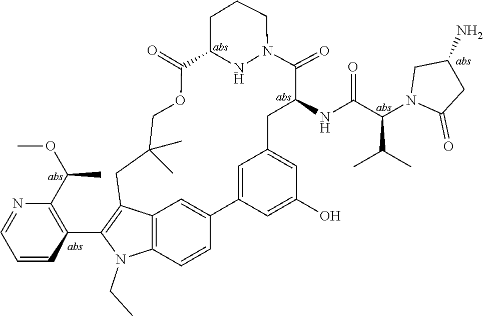 |
| AB 203 | 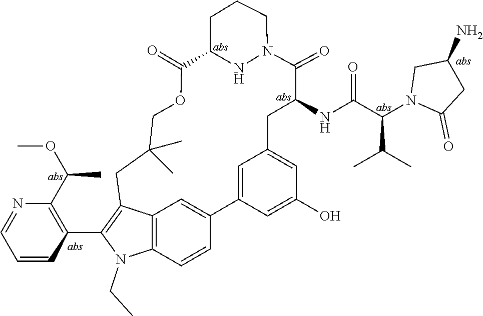 |
| AB 204 | 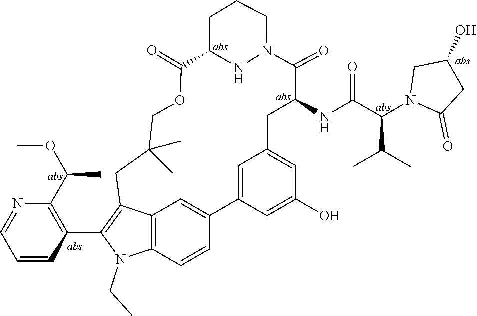 |
| AB 205 | 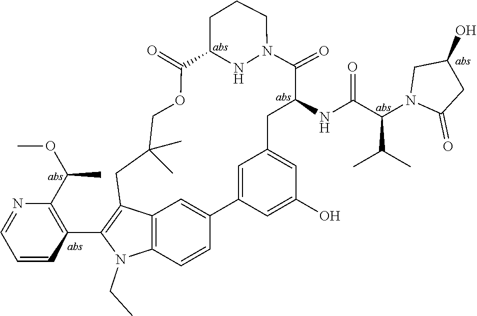 |
| AB 206 | 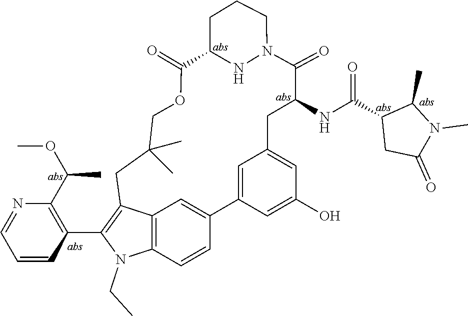 |
| AB 207 | 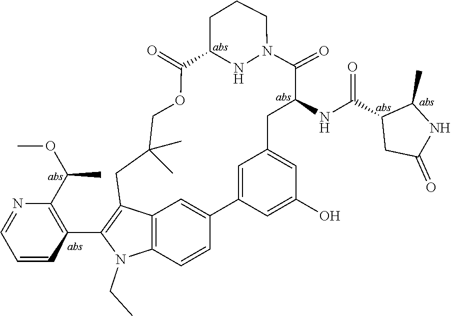 |
| AB 208 | 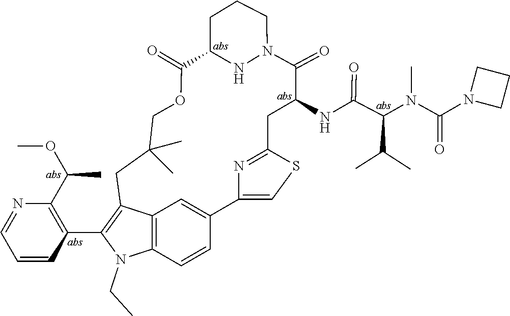 |
| AB 209 | 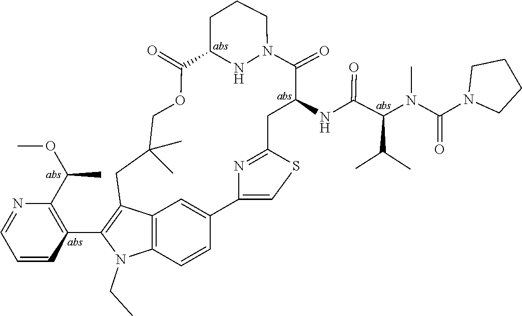 |
| AB 210 | 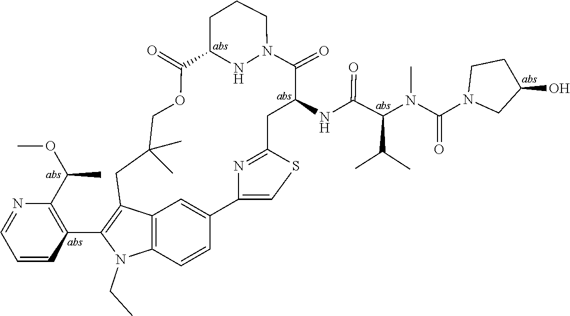 |
| AB 211 | 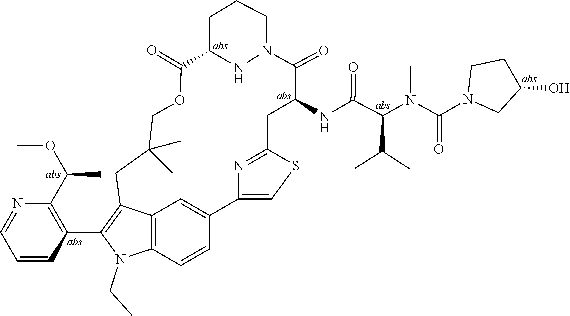 |
| AB 212 | 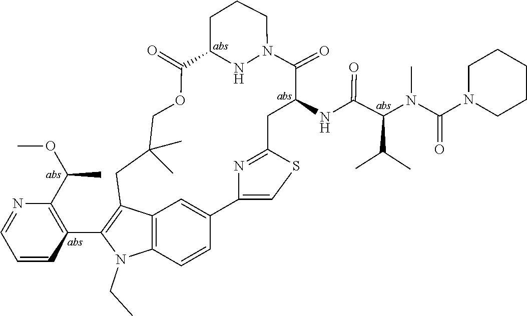 |
| AB 213 | 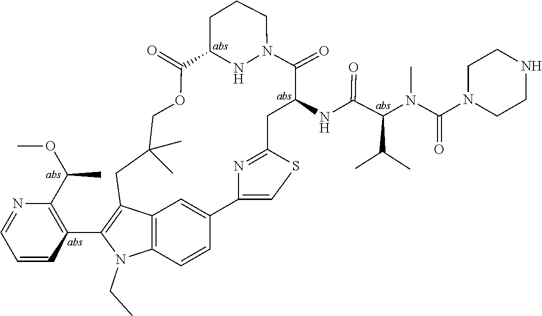 |
| AB 214 | 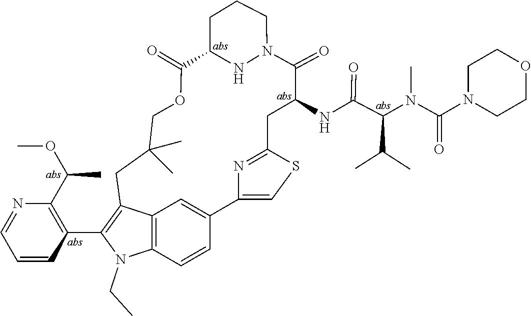 |
| AB 215 | 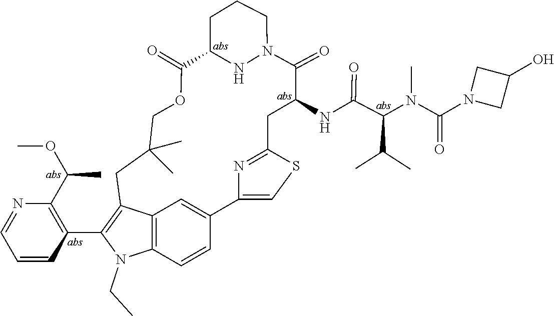 |
| AB 216 | 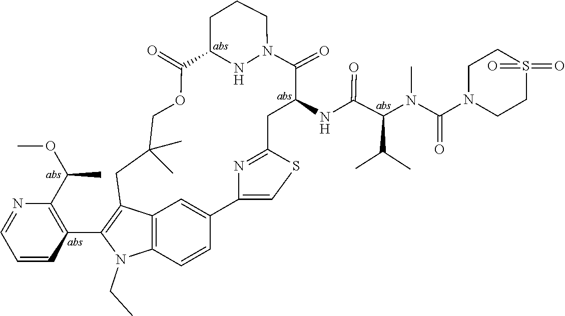 |
| AB 217 | 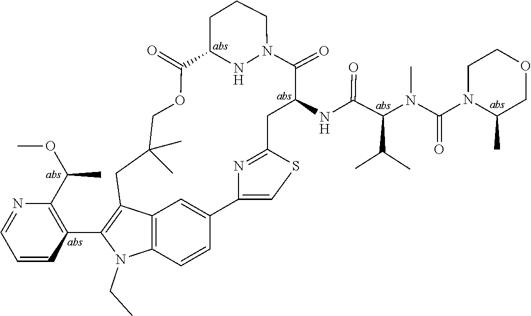 |
| AB 218 | 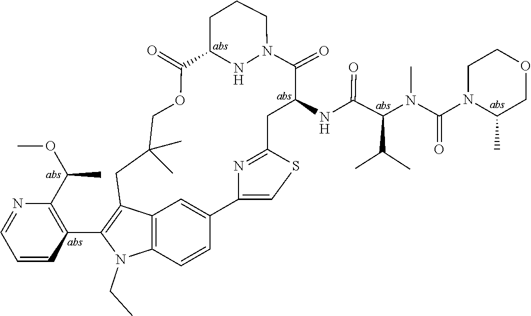 |
| AB 219 | 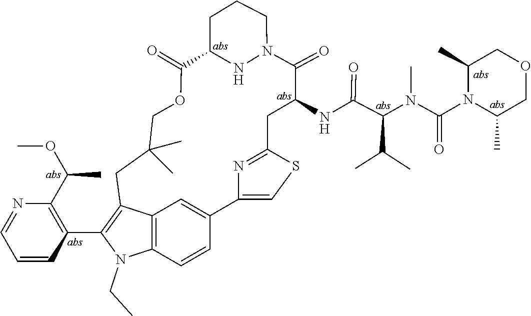 |
| AB 220 | 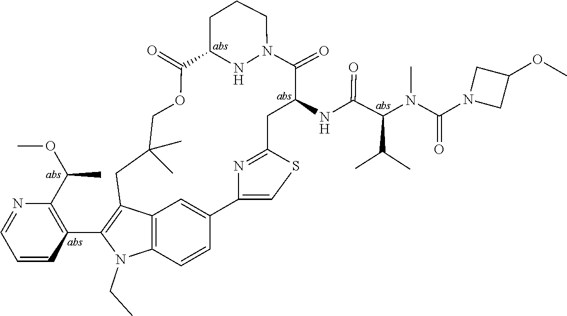 |
| AB 221 | 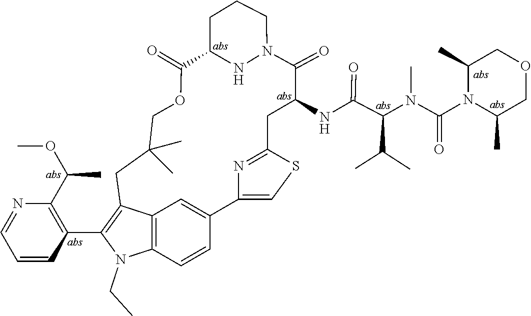 |
| AB 222 | 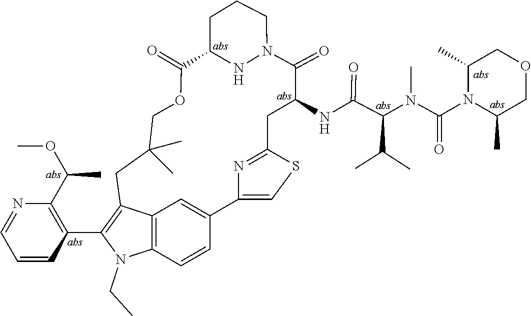 |
| AB 223 | 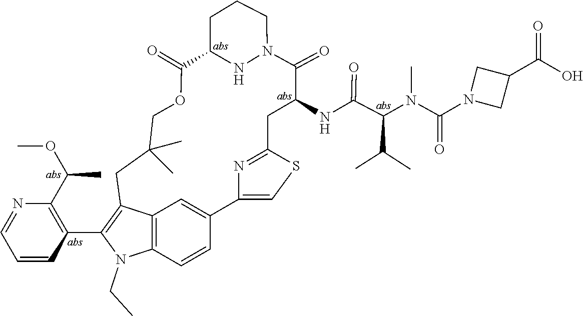 |
| AB 224 | 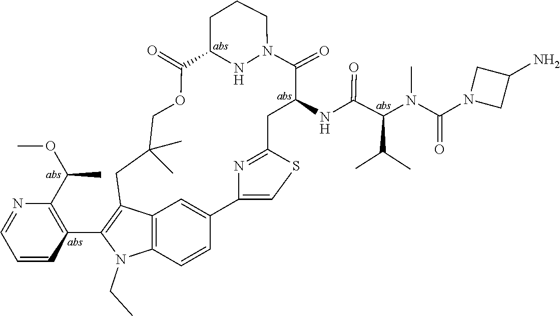 |
| AB 225 | 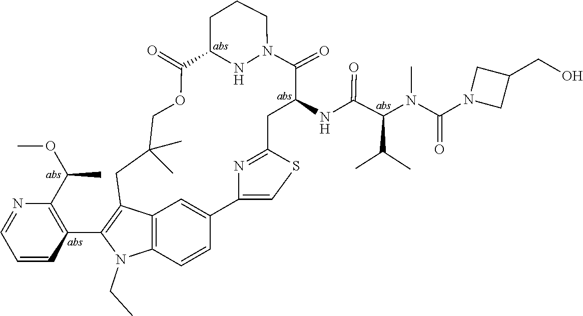 |
| AB 226 | 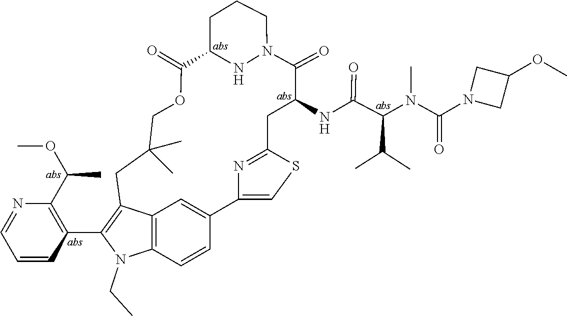 |
| AB 227 | 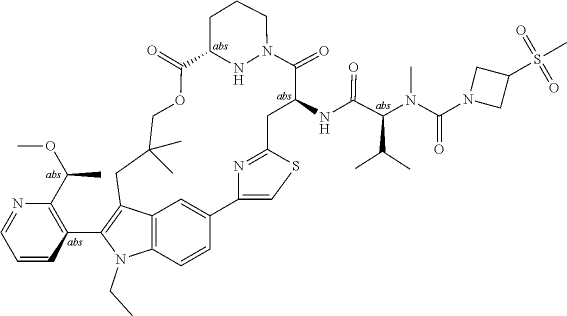 |
| AB 228 | 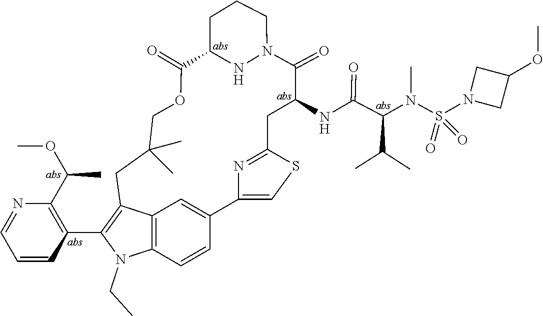 |
| AB 229 | 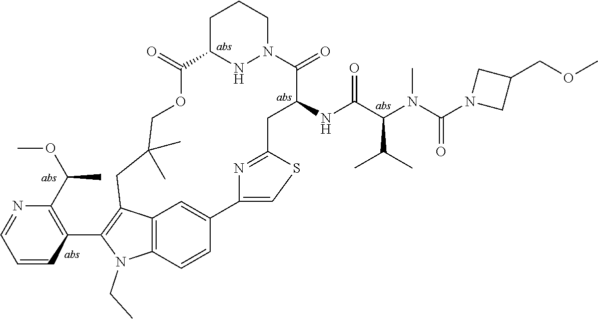 |
| AB 230 | 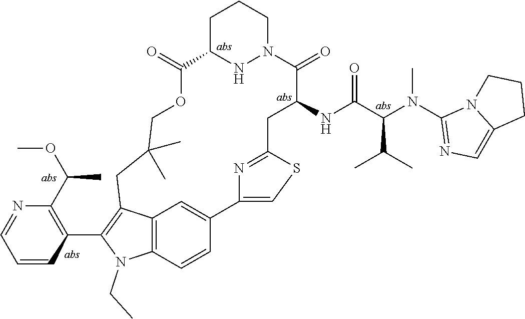 |
| AB 231 | 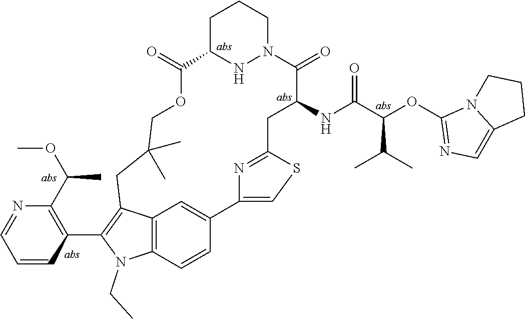 |
| AB 232 | 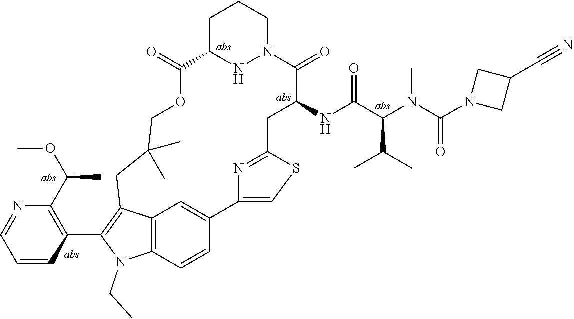 |
| AB 233 | 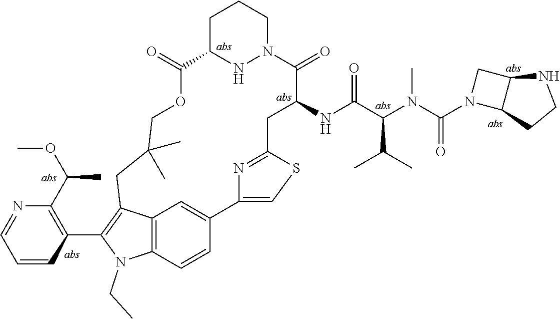 |
| AB 234 | 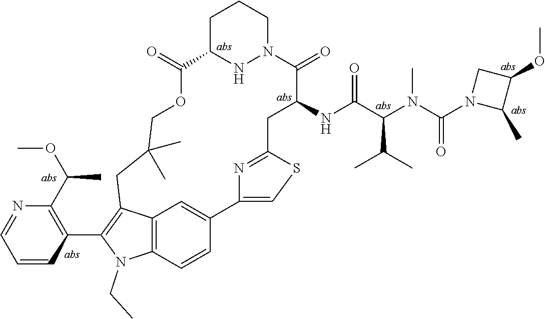 |
| AB 235 | 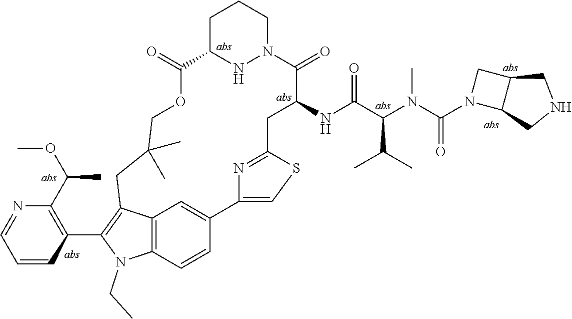 |
| AB 236 | 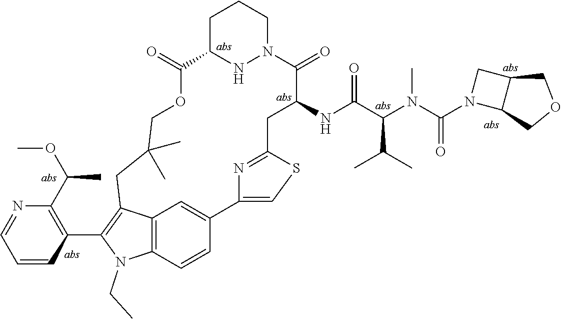 |
| AB 237 | 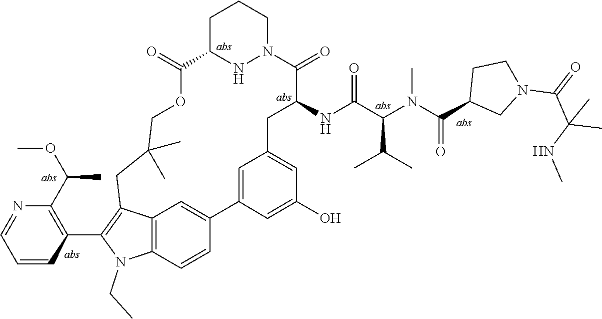 |
| AB 238 | 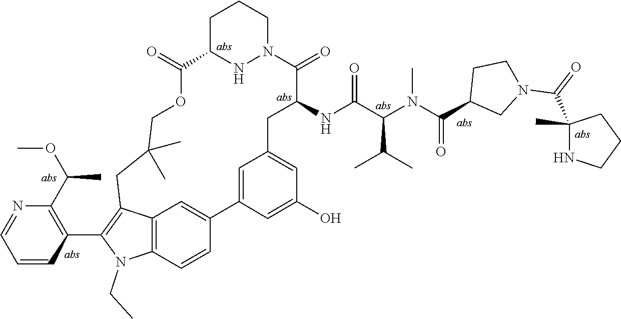 |
| AB 239 | 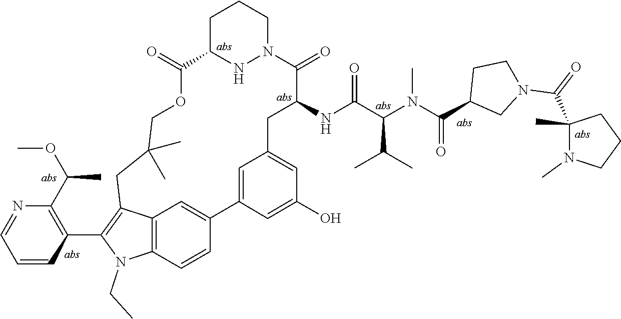 |
| AB 240 | 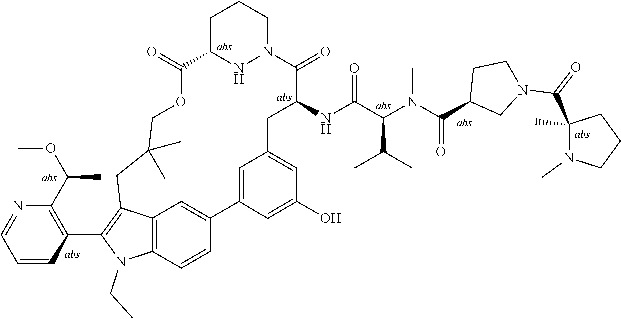 |
| AB 241 | 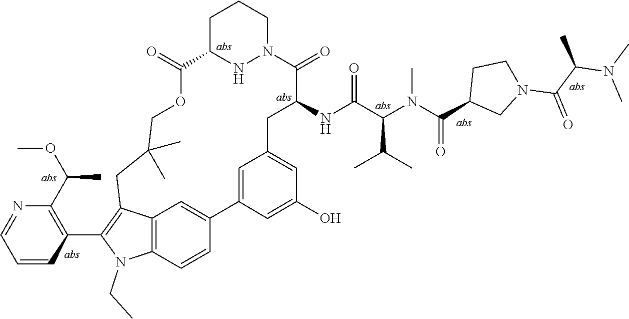 |
| AB 242 | 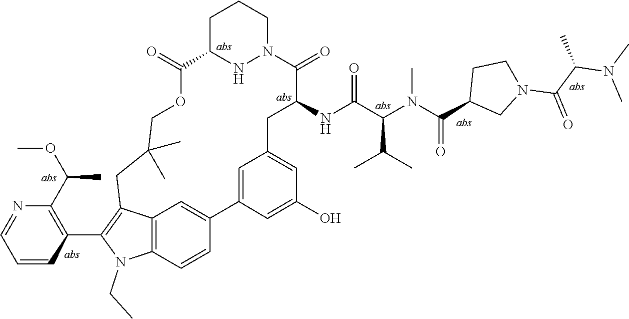 |
| AB 243 | 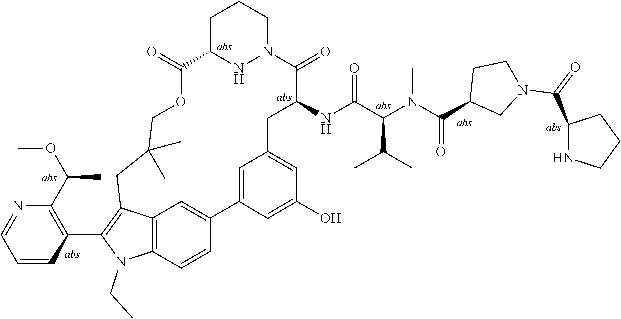 |
| AB 244 | 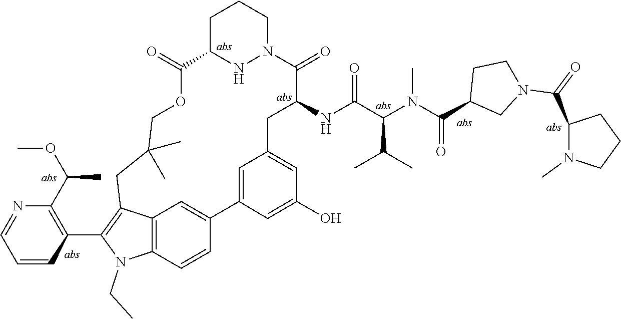 |
| AB 245 | 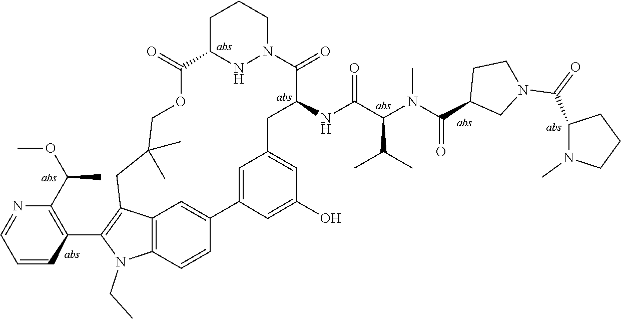 |
| AB 246 | 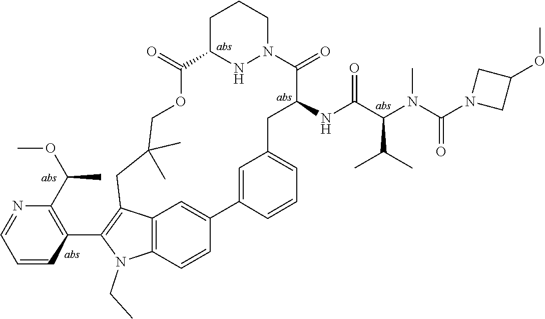 |
| AB 247 | 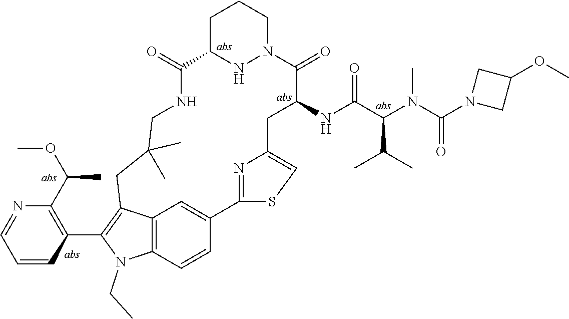 |
| AB 248 | 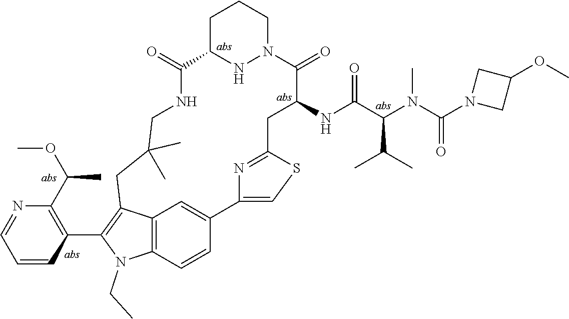 |
| AB 249 | 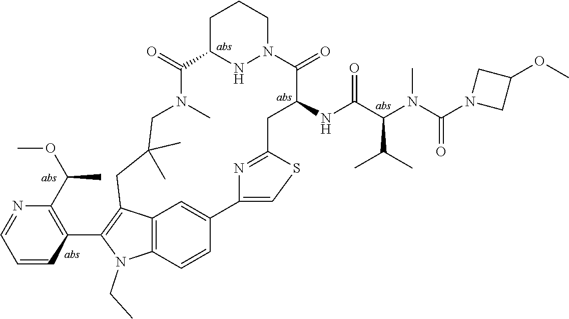 |
| AB 250 | 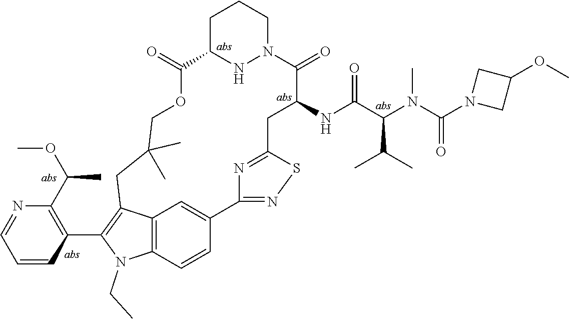 |
| AB 251 | 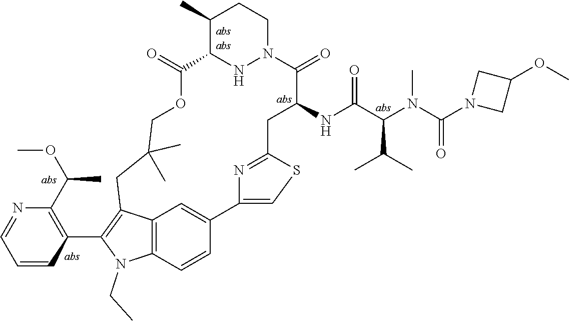 |
| AB 252 | 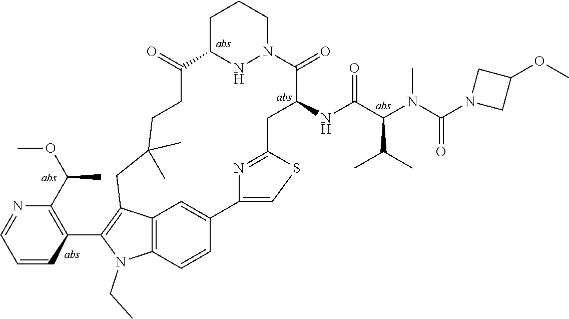 |
| AB 253 | 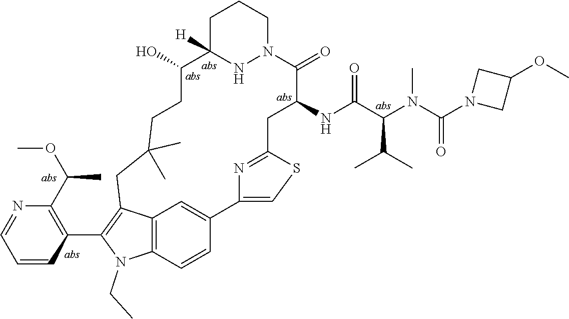 |
| AB 254 | 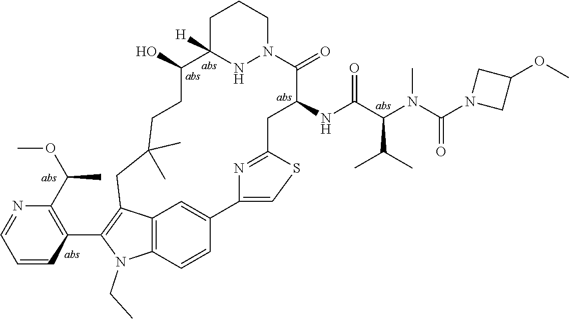 |
| AB 255 | 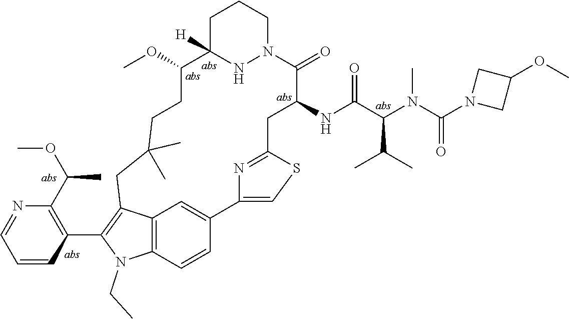 |
| AB 256 | 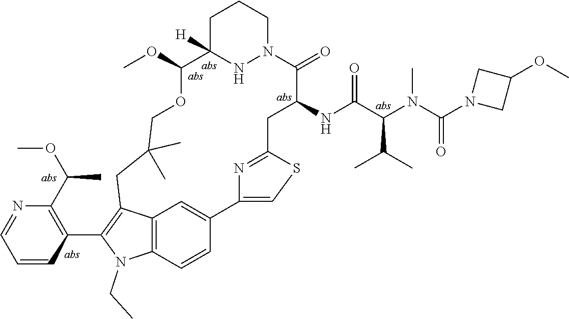 |
| AB 257 | 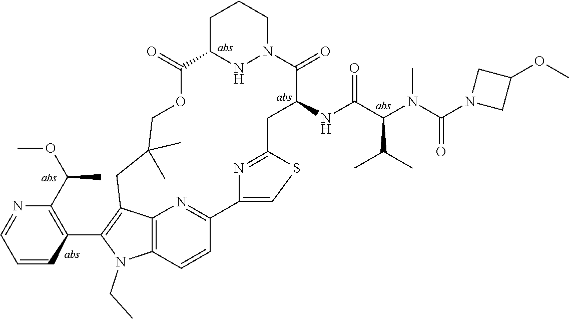 |
| AB 258 | 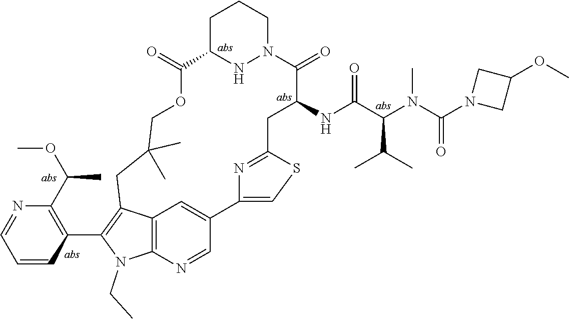 |
| AB 259 | 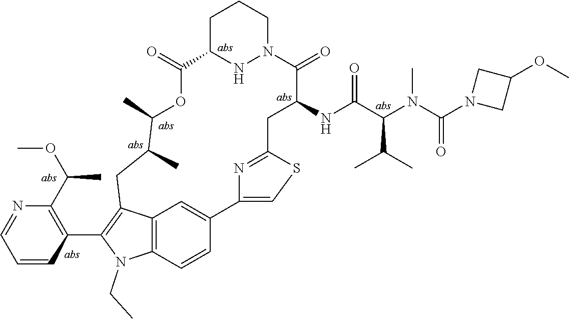 |
| AB 260 | 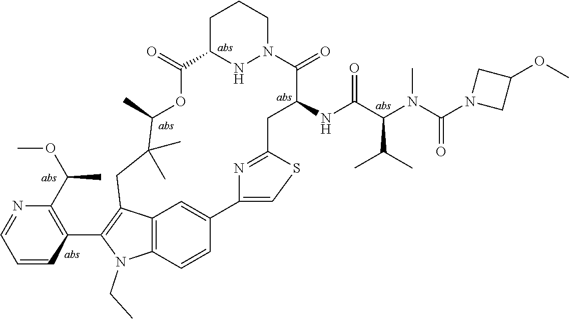 |
| AB 261 | 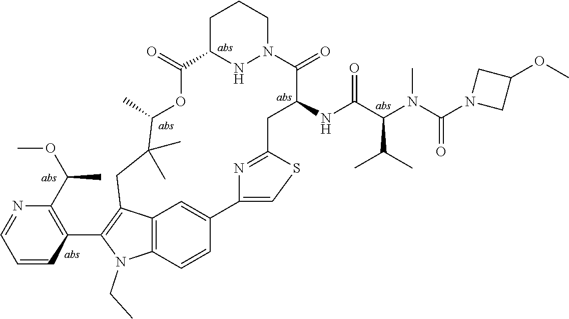 |
| AB 262 | 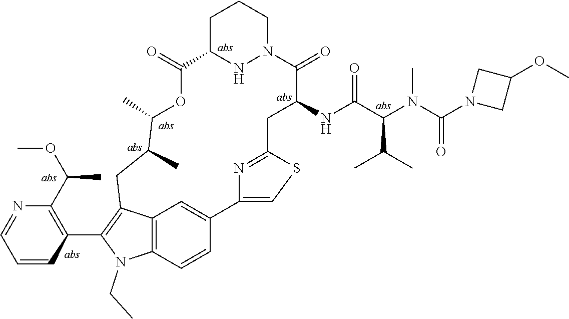 |
| AB 263 | 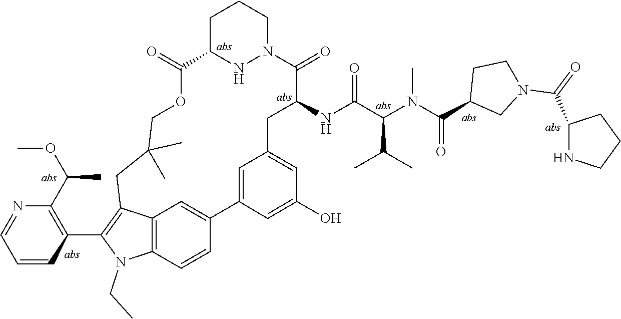 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below and in WO 2021/091956, together with synthetic methods known in the art of synthetic organic chemistry, or variations thereon as appreciated by those skilled in the art. These methods include but are not limited to those methods described in the Schemes below or as described in WO 2021/091956.

Compounds of Table A1 herein were prepared using methods disclosed herein or were prepared using methods disclosed herein combined with the knowledge of one of skill in the art. Compounds of Table A2 may be prepared using methods disclosed herein or may be prepared using methods disclosed herein combined with the knowledge of one of skill in the art.

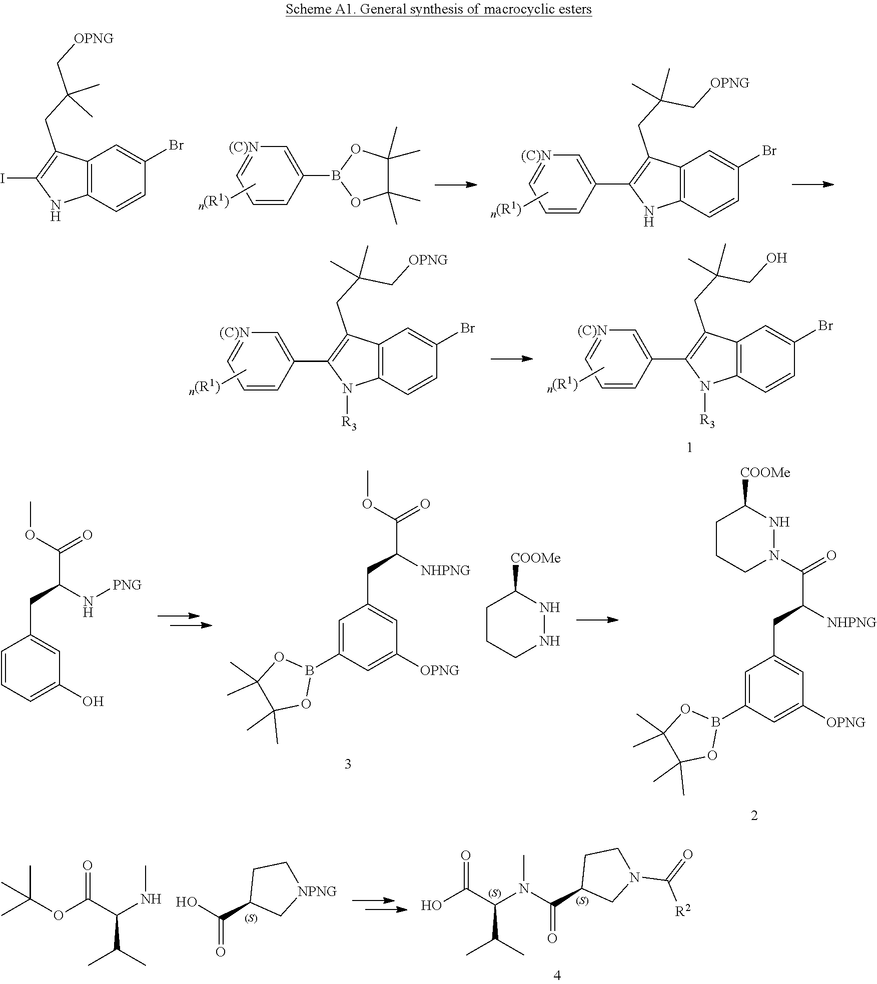

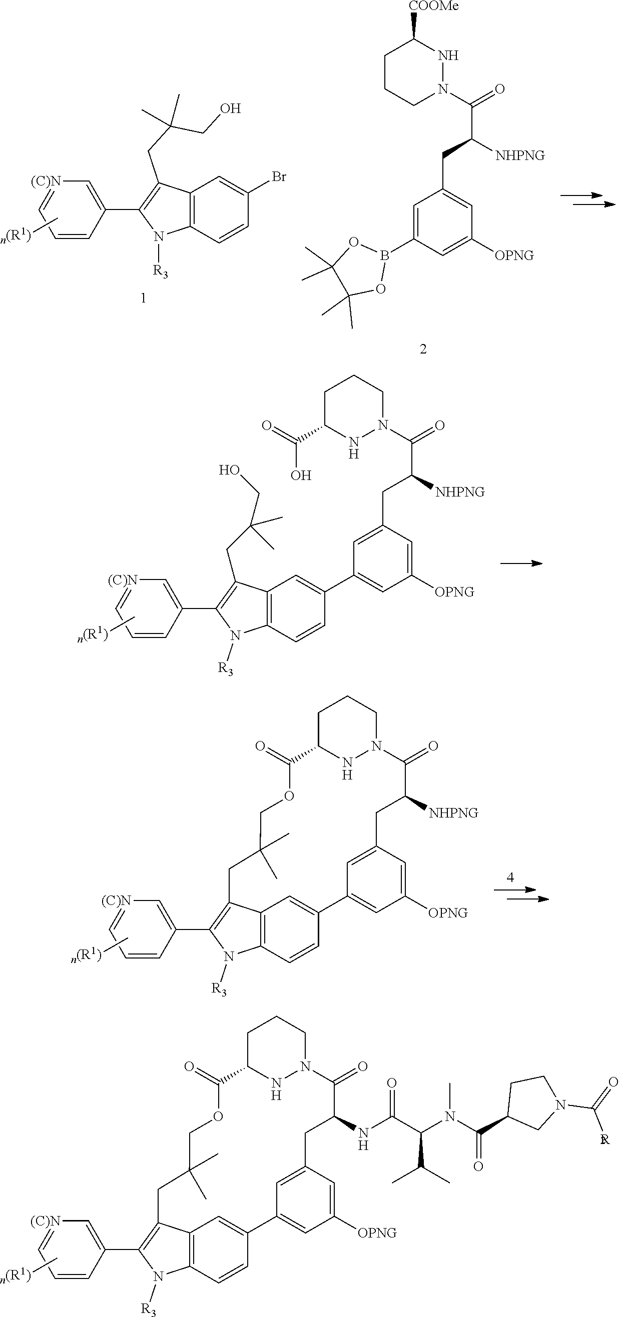

A general synthesis of macrocyclic esters is outlined in Scheme A1. An appropriately substituted Aryl Indole intermediate (1) can be prepared in three steps starting from protected 3-(5-bromo-2-iodo-1H-indol-3-yl)-2,2-dimethylpropan-1-ol and appropriately substituted boronic acid, including Palladium mediated coupling, alkylation, and de-protection reactions.

Methyl-amino-hexahydropyridazine-3-carboxylate-boronic ester (2) can be prepared in three steps, including protection, Iridium catalyst mediated borylation, and coupling with methyl (S)-hexahydropyridazine-3-carboxylate.

An appropriately substituted acetylpyrrolidine-3-carbonyl-N-methyl-L-valine (4) can be made by coupling of methyl-L-valinate and protected (S)-pyrrolidine-3-carboxylic acid, followed by deprotection, coupling with an appropriately substituted carboxylic acid, and a hydrolysis step.

The final macrocyclic esters can be made by coupling of methyl-amino-hexahydropyridazine-3-carboxylate-boronic ester (2) and intermediate (1) in the presence of Pd catalyst followed by hydrolysis and macrolactonization steps to result in an appropriately protected macrocyclic intermediate (5). Deprotection and coupling with an appropriately substituted acetylpyrrolidine-3-carbonyl-N-methyl-L-valine (4) results in a macrocyclic product. Additional deprotection or functionalization steps are be required to produce a final compound. For example, a person of skill in the art would be able to install into a macrocyclic ester a desired —B-L-W group of a compound of Formula (AI), where B, L and W are defined herein, including by using methods exemplified in the Example section herein.

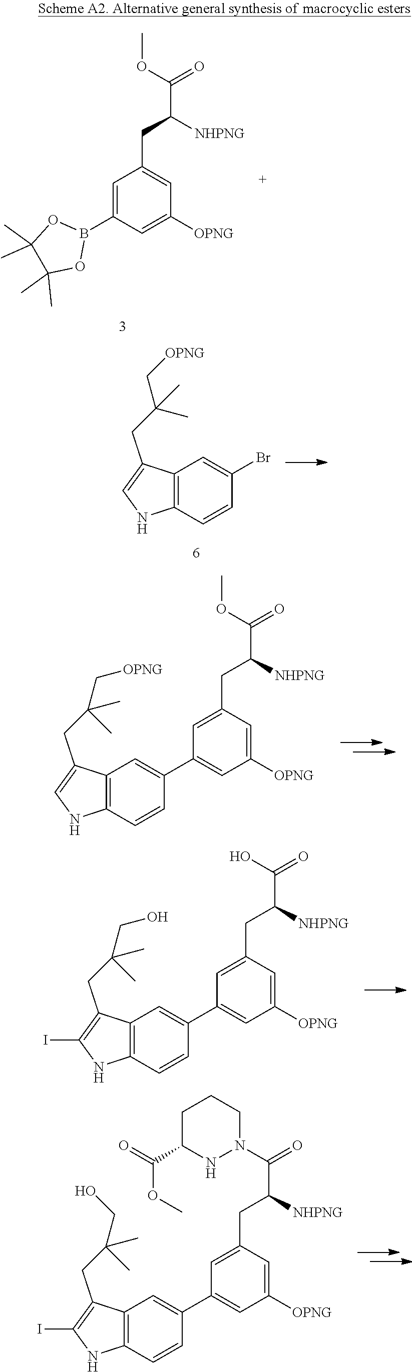

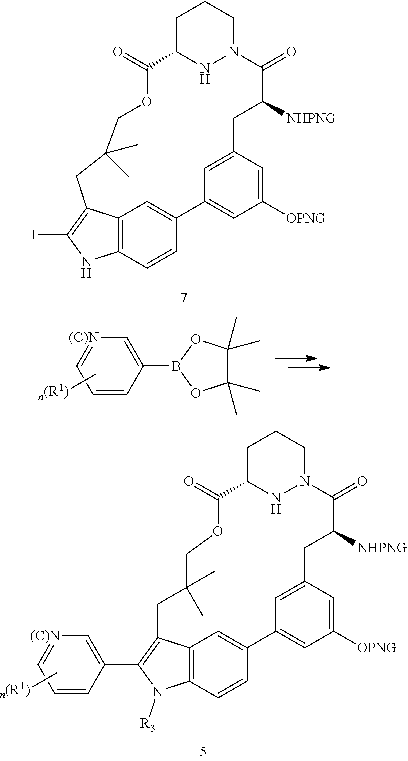

Alternatively, macrocyclic esters can be prepared as described in Scheme 2. An appropriately protected bromo-indolyl (6) can be coupled in the presence of Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of Pd catalyst with an appropriately substituted boronic ester and alkylation can yield fully a protected macrocycle (5). Additional deprotection or functionalization steps are required to produce a final compound. For example, a person of skill in the art would be able to install into a macrocyclic ester a desired —B-L-W group of a compound of Formula (AI), where B, L and W are defined herein, including by using methods exemplified in the Example section herein.

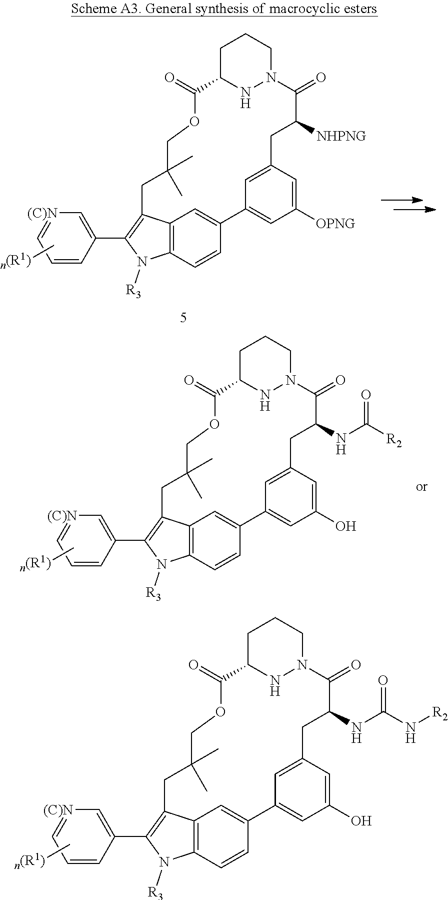

Alternatively, fully a protected macrocycle (5) can be deprotected and coupled with an appropriately substitututed coupling partners, and deprotected to results in a macrocyclic product. Additional deprotection or functionalization steps are be required to produce a final compound. For example, a person of skill in the art would be able to install into a macrocyclic ester a desired —B-L-W group of a compound of Formula (AI), where B, L and Ware defined herein, including by using methods exemplified in the Example section herein.

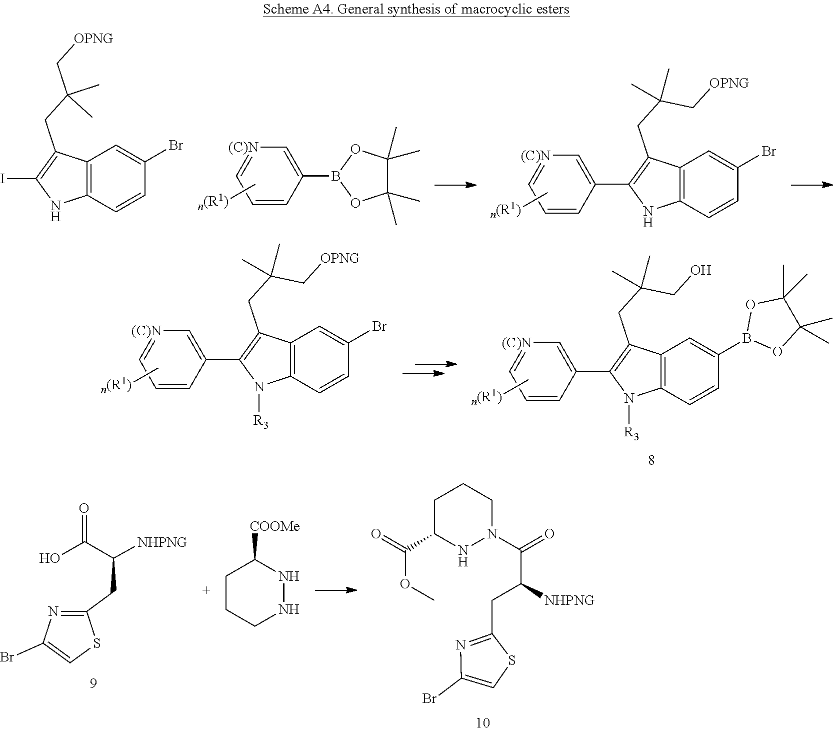

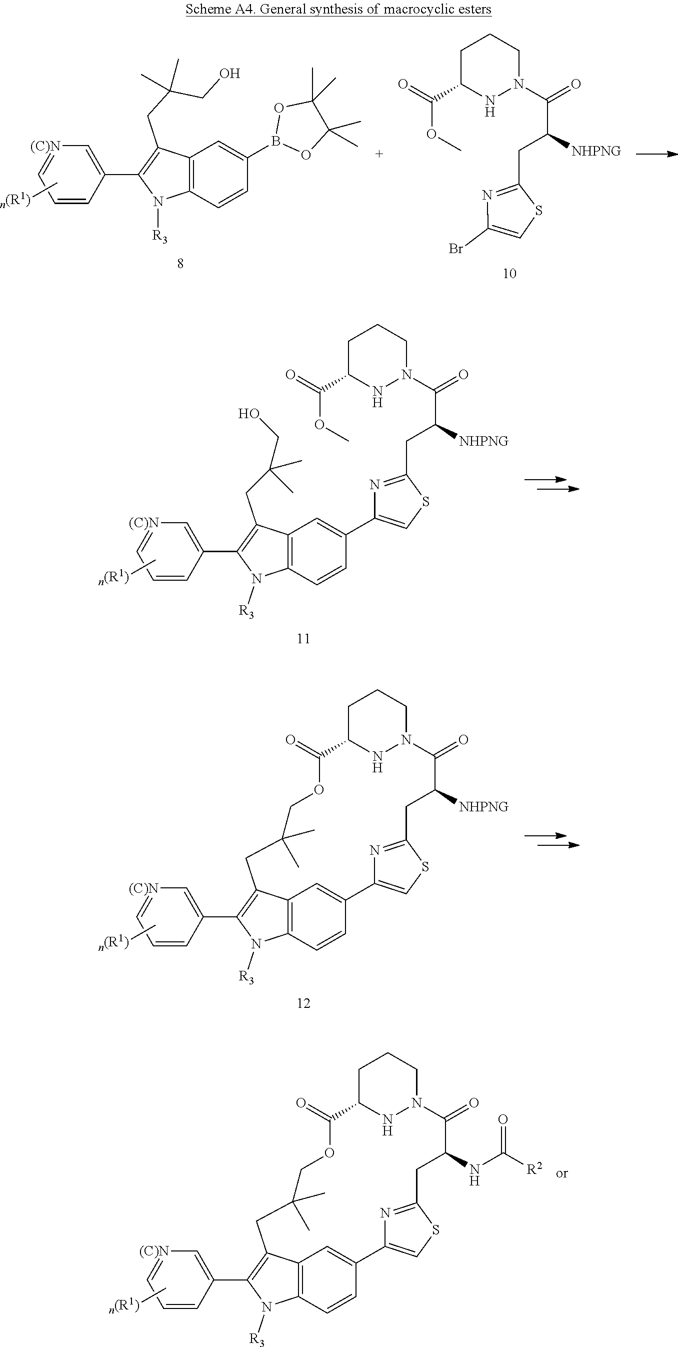

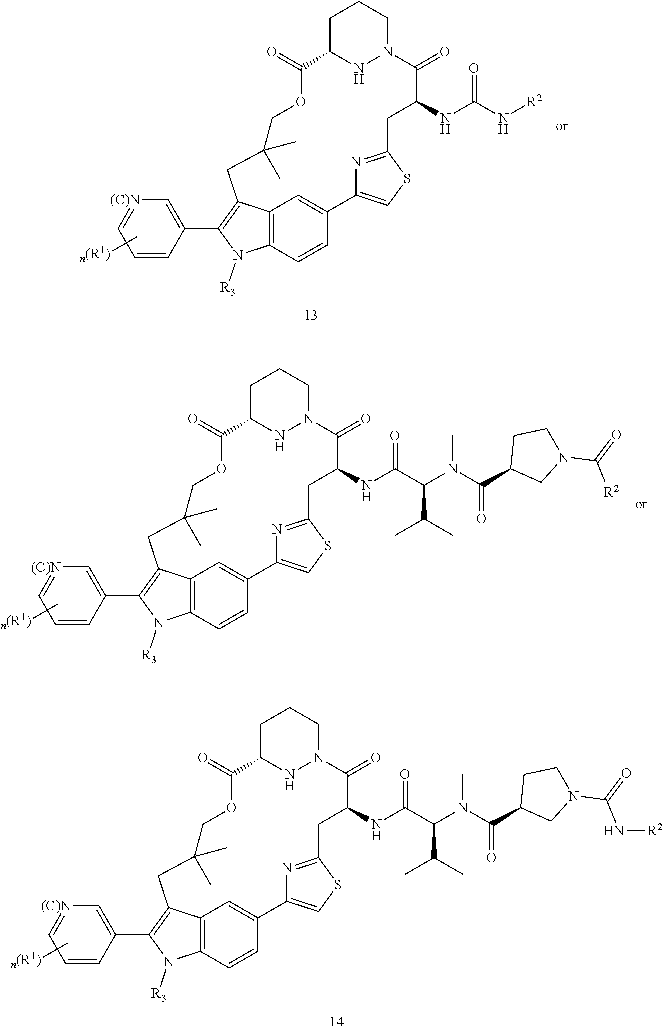

An alternative general synthesis of macrocyclic esters is outlined in Scheme A4. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted carboxylic acid (or other coupling partner) or intermediate 4 can result in a macrocyclic product. Additional deprotection or functionalization steps could be required to produce a final compound 13 or 14.

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the WO 2021/091956. For example, a person of skill in the art would be able to install into a macrocyclic ester a desired —B-L-W group of a compound of Formula (AI), where B, L and Ware defined herein, including by using methods exemplified in the Example section herein.

In some embodiments, the RAS(ON) inhibitor is a compound, or a pharmaceutically acceptable salt thereof, having the structure of Formula BI:

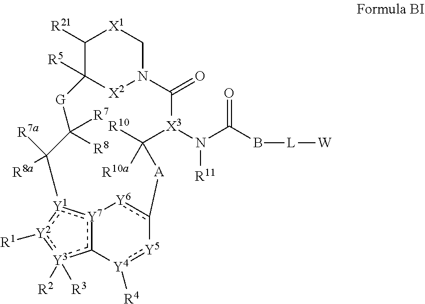

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R⁹′, or >CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments of Formula BI, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI, R²¹ is hydrogen.

In some embodiments, provided herein is a compound, or pharmaceutically acceptable salt thereof, having the structure of Formula BIa:

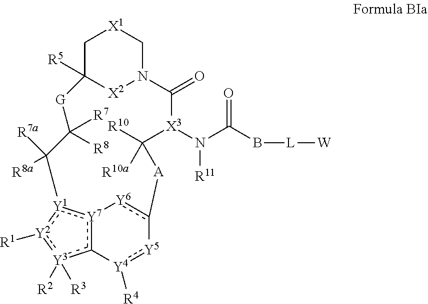

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone; X¹ is optionally substituted C₁-C₂ alkylene, NR, 0, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, (O)R, C(O)OR, C(O)N(R)₂, S(O)R, S(O)₂R, or S(O)₂N(R′)₂;
each R′ is, independently, H or optionally substituted C₁-C₄ alkyl;
Y¹ is C, CH, or N;
Y², Y³, Y⁴ and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the disclosure features a compound, or pharmaceutically acceptable salt thereof, of structural Formula BIb:

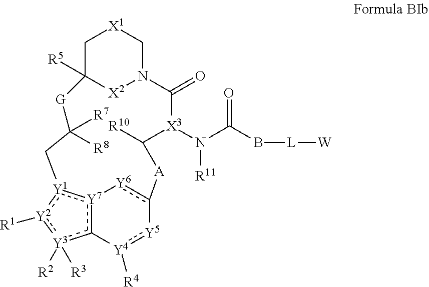

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula BI and subformula thereof, G is optionally substituted C₁-C₄ heteroalkylene.

In some embodiments, a compound having the structure of Formula BIc is provided, or a pharmaceutically acceptable salt thereof:

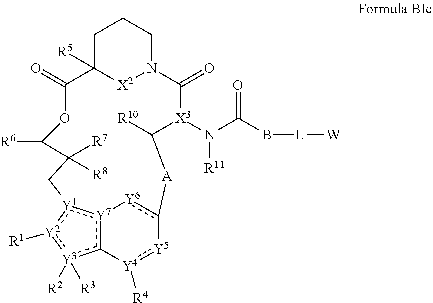

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula BI and subformula thereof, X² is NH. In some embodiments of Formula BI and subformula thereof, X³ is CH. In some embodiments of Formula BI and subformula thereof, R¹¹ is hydrogen. In some embodiments of Formula BI and subformula thereof, R¹¹ is C₁-C₃ alkyl.

In some embodiments of Formula BI and subformula thereof, R¹¹ is methyl.

In some embodiments, the RAS(ON) inhibitor has the structure of Formula BId, or a pharmaceutically acceptable salt thereof:

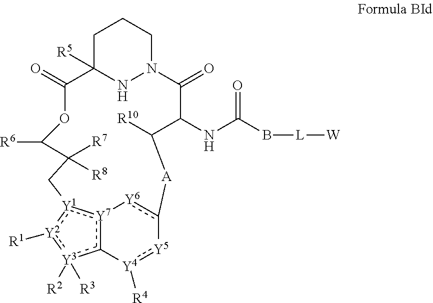

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula BI and subformula thereof, X¹ is optionally substituted C₁-C₂ alkylene. In some embodiments, X¹ is methylene. In some embodiments of Formula BI and subformula thereof, X¹ is methylene substituted with a C₁-C₆ alkyl group or a halogen. In some embodiments, X¹ is —CH(Br)—. In some embodiments, X¹ is —CH(CH₃)—. In some embodiments of Formula BI and subformula thereof, R⁵ is hydrogen. In some embodiments of Formula BI and subformula thereof, R⁵ is C₁-C₄ alkyl optionally substituted with halogen. In some embodiments, R⁵ is methyl. In some embodiments of Formula BI and subformula thereof, Y⁴ is C. In some embodiments of Formula BI and subformula thereof, R⁴ is hydrogen. In some embodiments of Formula BI and subformula thereof, Y⁵ is CH. In some embodiments of Formula BI and subformula thereof, Y⁶ is CH. In some embodiments of Formula BI and subformula thereof, Y¹ is C. In some embodiments of Formula BI and subformula thereof, Y² is C. In some embodiments of Formula BI and subformula thereof, Y³ is N. In some embodiments of Formula BI and subformula thereof, R³ is absent. In some embodiments of Formula BI and subformula thereof, Y⁷ is C.

In some embodiments, the RAS(ON) inhibitor has the structure of Formula BIe, or a pharmaceutically acceptable salt thereof:

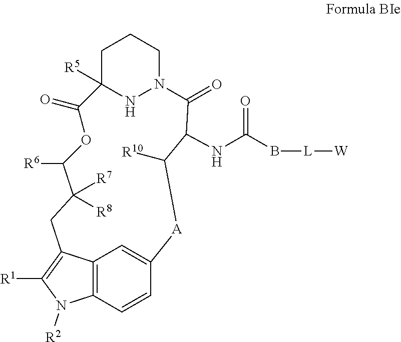

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula BI and subformula thereof, R⁶ is hydrogen. In some embodiments, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl. In some embodiments, R² is fluoroalkyl. In some embodiments, R² is ethyl. In some embodiments, R² is —CH₂CF₃. In some embodiments, R² is C₂-C₆ alkynyl. In some embodiments, R² is —CHC≡CH. In some embodiments, R² is —CH₂C≡CCH₃. In some embodiments, R⁷ is optionally substituted C₁-C₃ alkyl. In some embodiments, R⁷ is C₁-C₃ alkyl. In some embodiments, R⁸ is optionally substituted C₁-C₃ alkyl. In some embodiments, R⁸ is C₁-C₃ alkyl.

In some embodiments, the RAS(ON) inhibitor has the structure of Formula BIf, or a pharmaceutically acceptable salt thereof:

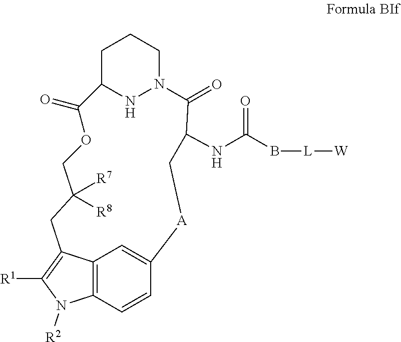

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI and subformula thereof, R¹ is optionally substituted 6 to 10-membered aryl, optionally substituted 3 to 6-membered cycloalkenyl, or optionally substituted 5 to 10-membered heteroaryl. In some embodiments, R¹ is optionally substituted 6-membered aryl, optionally substituted 6-membered cycloalkenyl, or optionally substituted 6-membered heteroaryl.

In some embodiments of Formula BI and subformula thereof, R₁ is

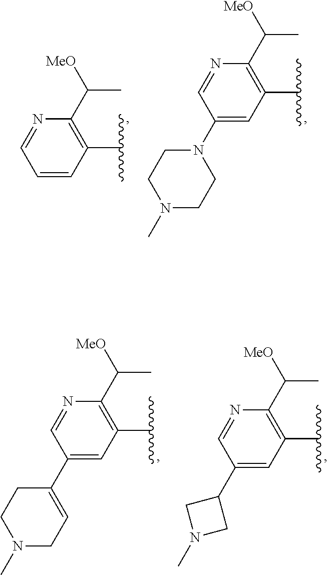

In some embodiments of Formula BI and subformula thereof, R¹² is optionally substituted C₁-C₆ heteroalkyl. In some embodiments, R¹² is

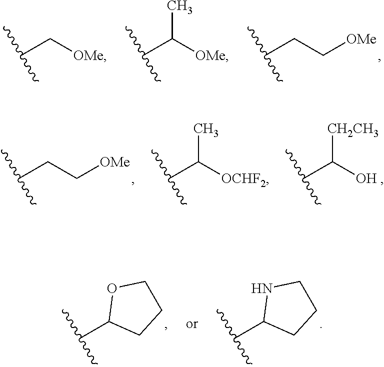

In some embodiments, R¹² is

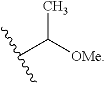 [SMILES: *C(C)OC]

In some embodiments, the RAS(ON) inhibitor has the structure of Formula BVI, or a pharmaceutically acceptable salt thereof:

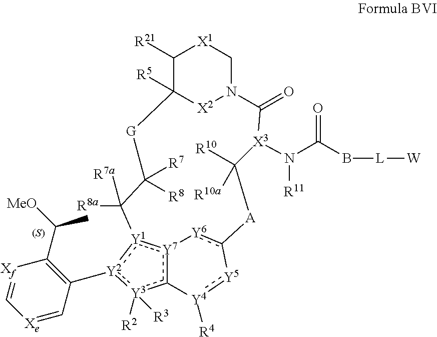

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is absent, —CH(R⁹)—, >C═CR⁹R⁹′, or >CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R²¹ is hydrogen or C₁-C₃ alkyl (e.g., methyl); and
X^{e} and X^{f} are, independently, N or CH.

In some embodiments, the RAS(ON) inhibitor has the structure of Formula BVIa, or a pharmaceutically acceptable salt thereof:

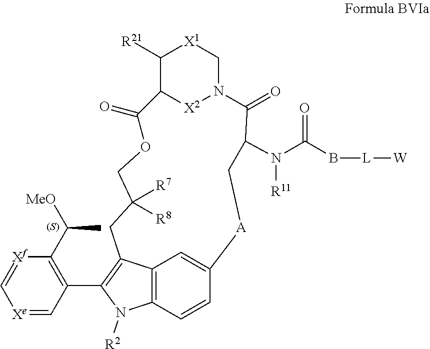

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a vinyl ketone, a vinyl sulfone, an ynone, or an alkynyl sulfone;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of Formula BI and subformula thereof, X^{e} is N and X^{f} is CH. In some embodiments, X^{e} is CH and X^{f} is N.

In some embodiments, the RAS(ON) inhibitor has the structure of Formula BVIb, or a pharmaceutically acceptable salt thereof:

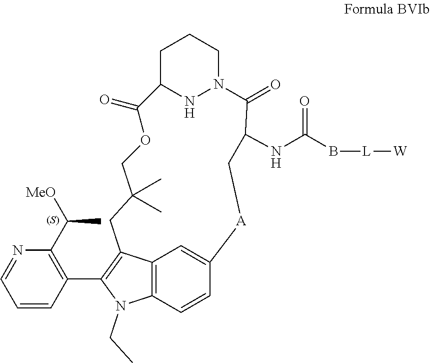

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
L is absent or a linker; and
W is a cross-linking group comprising a vinyl ketone, a vinyl sulfone, an ynone, or an alkynyl sulfone.

In some embodiments of formula BI or subformula thereof, A is optionally substituted 6-membered arylene.

In some embodiments, the RAS(ON) inhibitor has the structure of Formula BVIc, or a pharmaceutically acceptable salt thereof:

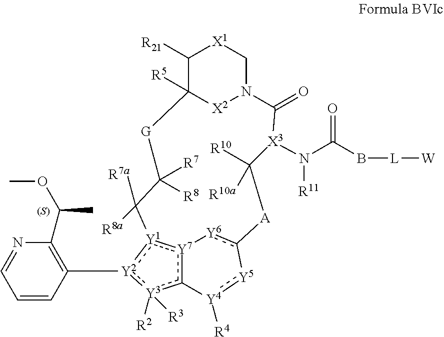

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is absent, —CH(R⁹)—, >C═CR⁹R⁹′, or >CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl (e.g., methyl).

In some embodiments of Formula BI and subformula thereof, A has the structure:

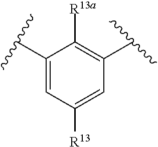

wherein R¹³ is hydrogen, halo, hydroxy, amino, optionally substituted C₁-C₆ alkyl, or optionally substituted C₁-C₆ heteroalkyl; and R^{13a} is hydrogen or halo. In some embodiments, R¹³ is hydrogen. In some embodiments, R¹³ and R^{13a} are each hydrogen. In some embodiments, R¹³ is hydroxy, methyl, fluoro, or difluoromethyl.

In some embodiments of Formula BI and subformula thereof, A is optionally substituted 5 to 6-membered heteroarylene. In some embodiments, A is:

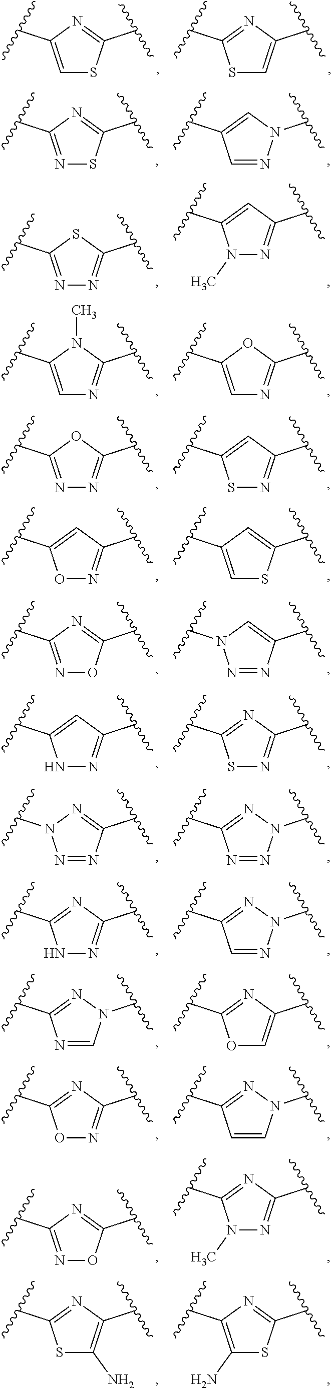

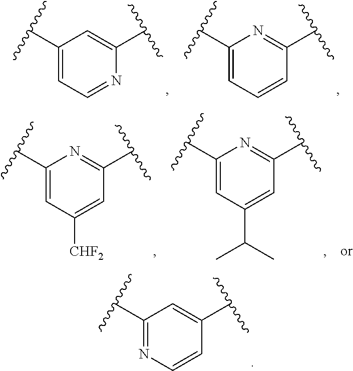

In some embodiments of Formula BI and subformula thereof, A is optionally substituted C₁-C₄ heteroalkylene. In some embodiments, A is:

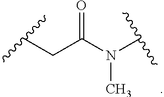 [SMILES: *CC(=O)N(*)C]

In some embodiments of Formula BI and subformula thereof, A is optionally substituted 3 to 6-membered heterocycloalkylene. In some embodiments, A is:

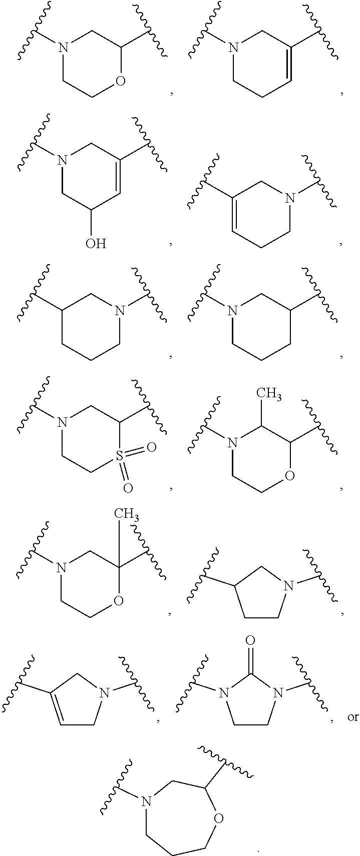

In some embodiments, A is

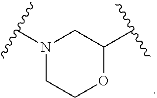 [SMILES: *C1CN(*)CCO1]

In some embodiments of Formula BI and subformula thereof, B is —CHR⁹—. In some embodiments of Formula BI and subformula thereof, R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl. In some embodiments, R⁹ is:

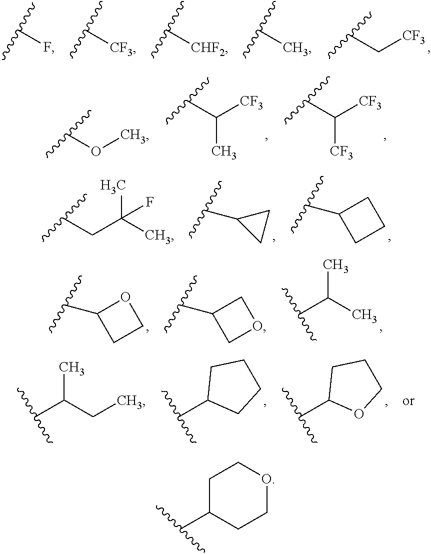

In some embodiments, R⁹ is:

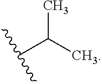 [SMILES: *C(C)C]

In some embodiments, R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula BI and subformula thereof, B is optionally substituted 6-membered arylene. In some embodiments, B is 6-membered arylene. In some embodiments, B is:

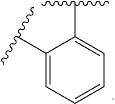 [SMILES: *c1ccccc1*]

In some embodiments of Formula BI and subformula thereof, R⁷ is methyl.

In some embodiments of Formula BI and subformula thereof, R⁸ is methyl.

In some embodiments of Formula BI and subformula thereof, R²¹ is hydrogen.

In some embodiments of Formula BI and subformula thereof, the linker is the structure of Formula BII:

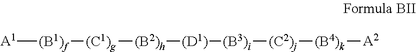

where A¹ is a bond between the linker and B; A² is a bond between Wand the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁₋₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C₁ and C₂ are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments, the linker is acyclic. In some embodiments, linker has the structure of Formula BIIa:

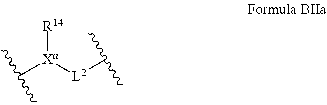

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

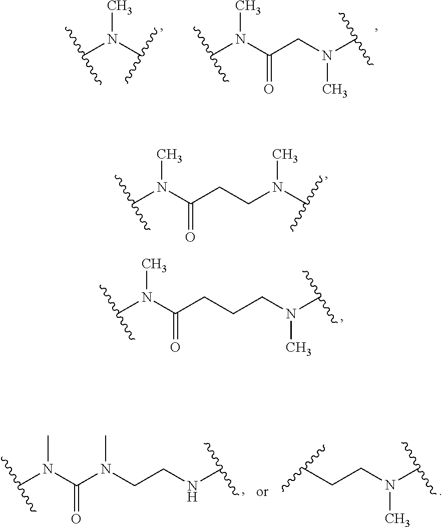

In some embodiments of Formula BI and subformula thereof, the linker is or comprises a cyclic moiety. In some embodiments, the linker has the structure of Formula BIIb:

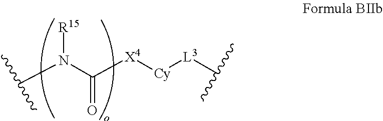

wherein o is 0 or 1;
R¹⁵ is hydrogen or optionally substituted C₁-C₆ alkyl, optionally substituted 3 to 8-membered cycloalkylene, or optionally substituted 3 to 8-membered heterocycloalkylene;
X⁴ is absent, optionally substituted C₁-C₄ alkylene, O, NCH₃, or optionally substituted C₁-C₄ heteroalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.

In some embodiments of Formula BI and subformula thereof, the linker has the structure of Formula BIIb-1:

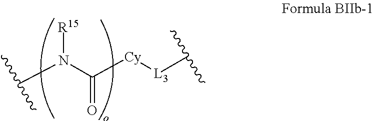

wherein o is 0 or 1;
R¹⁵ is hydrogen or optionally substituted C₁-C₆ alkyl, optionally substituted 3 to 8-membered cycloalkylene, or optionally substituted 3 to 8-membered heterocycloalkylene;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene.

In some embodiments of Formula BI and subformula thereof, the linker has the structure of Formula BIIc:

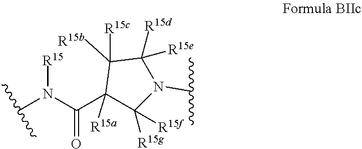

wherein R¹⁵ is hydrogen, optionally substituted C₁-C₆ alkyl, optionally substituted 3 to 8-membered cycloalkylene, or optionally substituted 3 to 8-membered heterocycloalkylene; and
R^{15a}, R^{15b}, R^{15c}, R^{15d}, R^{15e}, R^{15f}, and R¹⁵9 are, independently, hydrogen, halo, hydroxy, cyano, amino, optionally substituted C₁-C₆ alkyl, optionally substituted C₁-C₆ alkoxy, or, or R^{15b} and R^{15d} combine with the carbons to which they are attached to form an optionally substituted 3 to 8-membered cycloalkylene, or optionally substituted 3 to 8-membered heterocycloalkylene.

In some embodiments of Formula BI and subformula thereof, the linker has the structure:

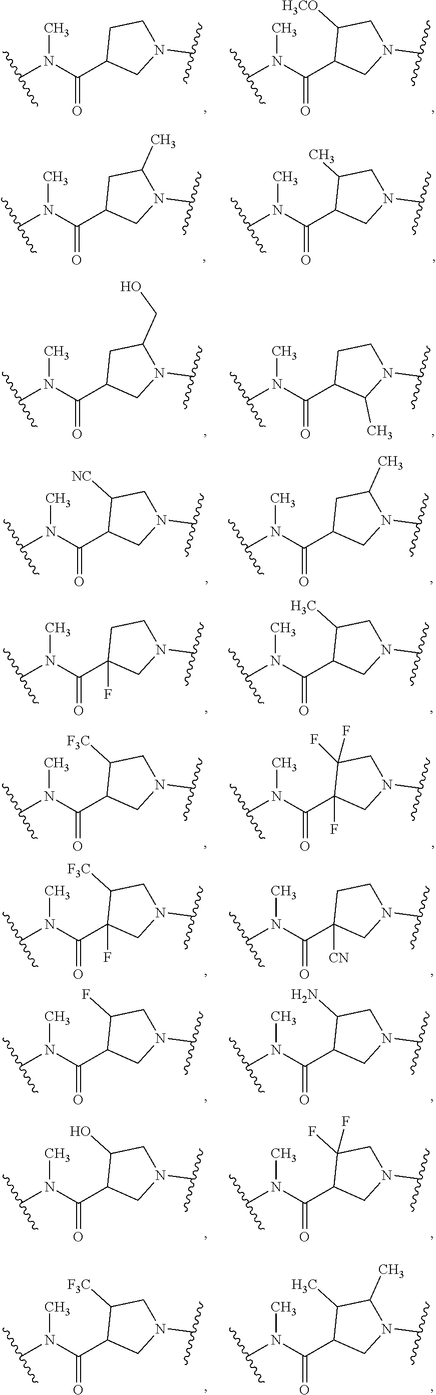

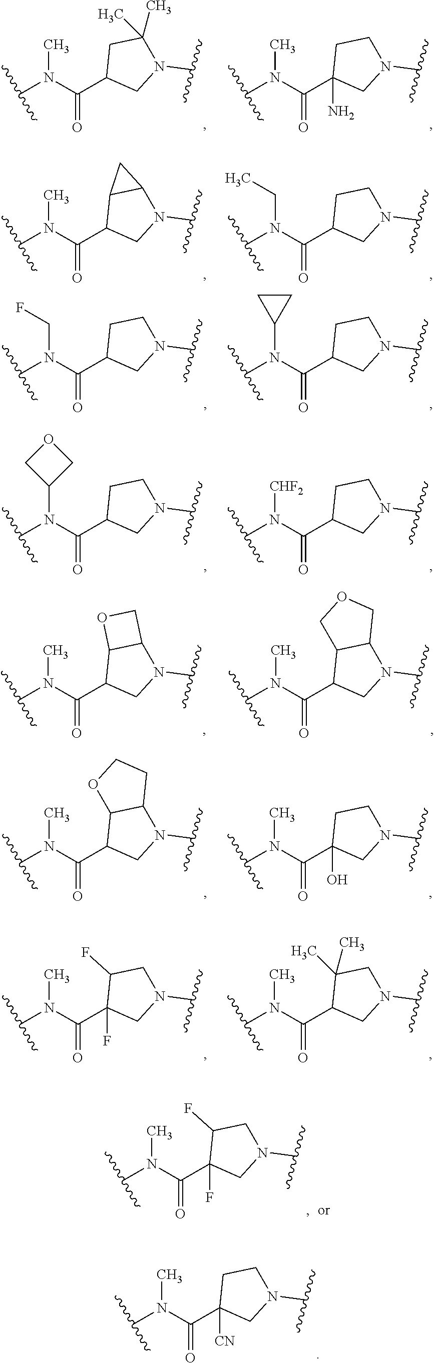

In some embodiments of Formula BI and subformula thereof, the linker has the structure:

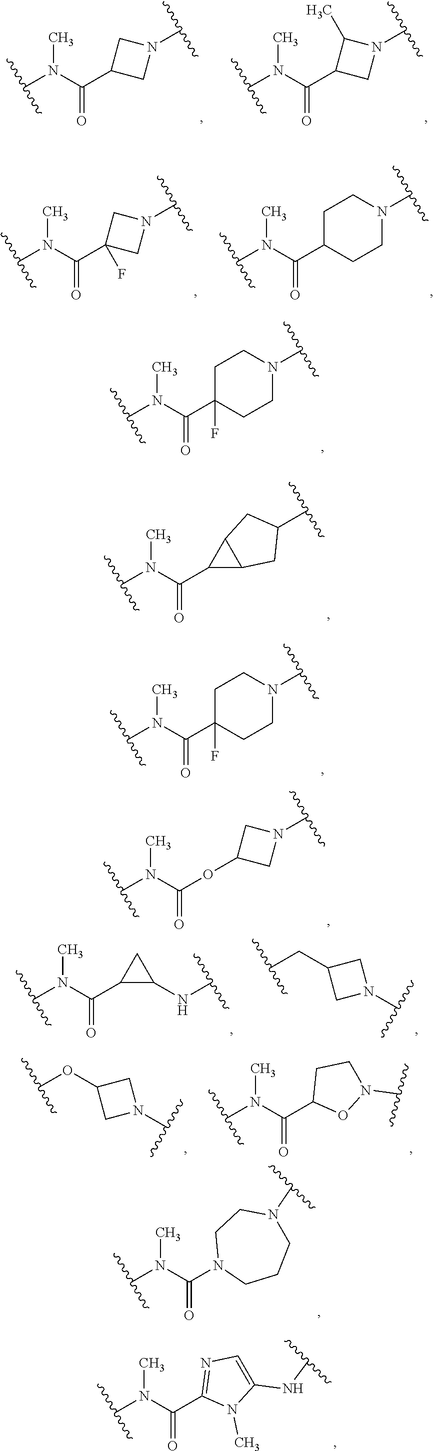

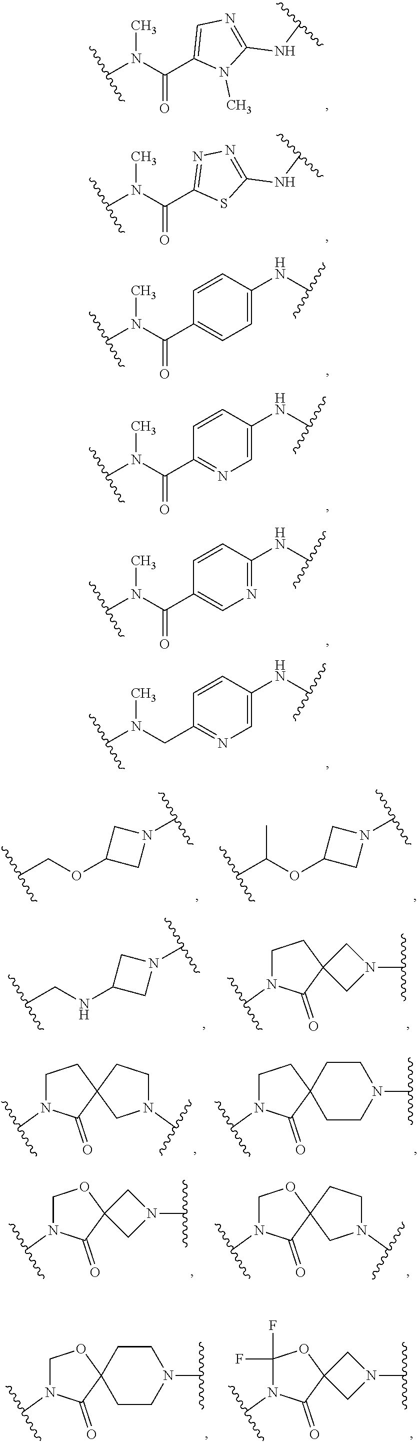

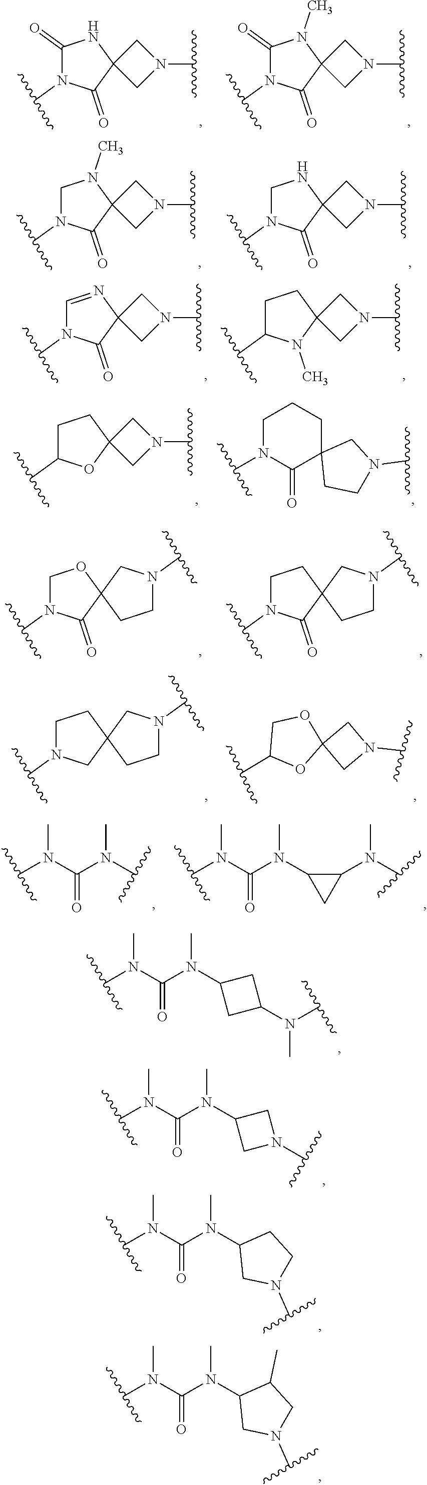

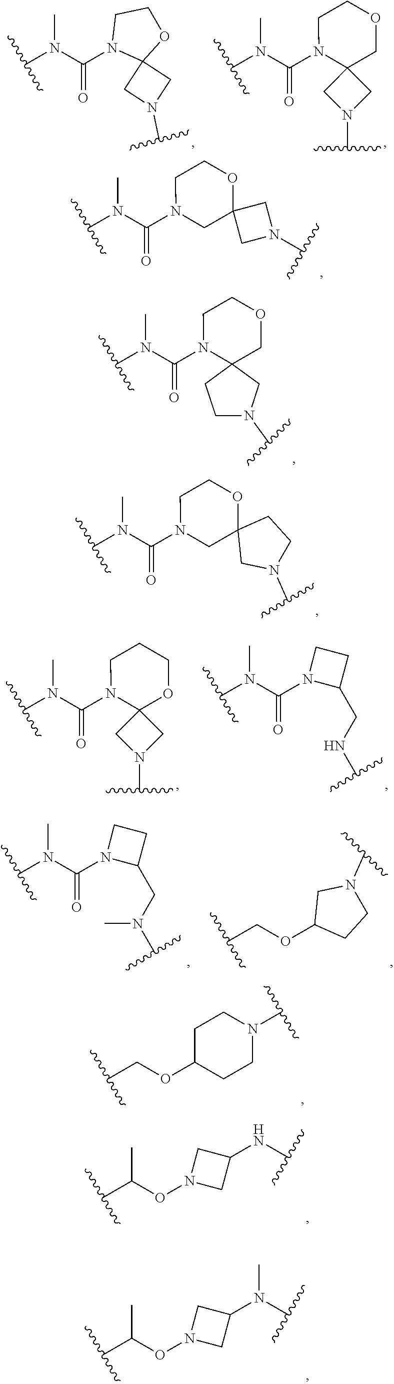

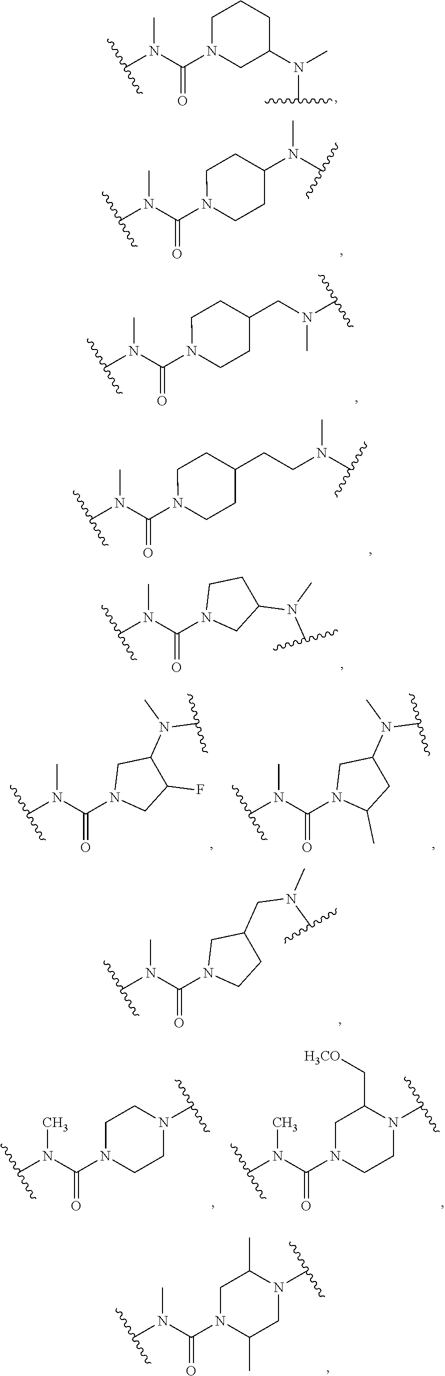

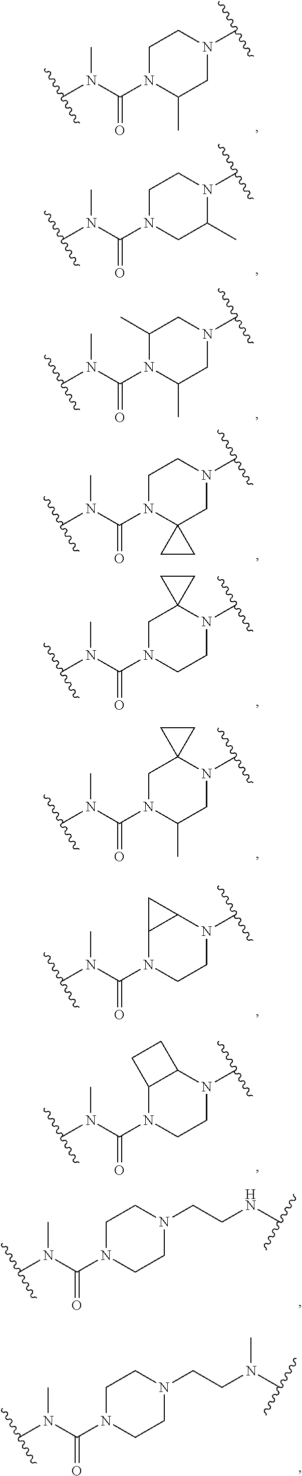

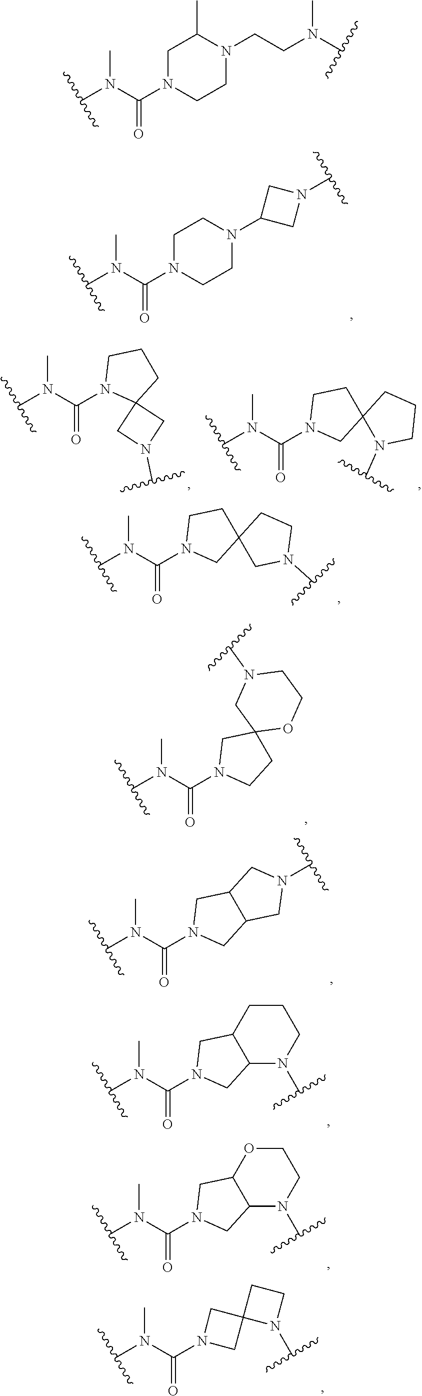

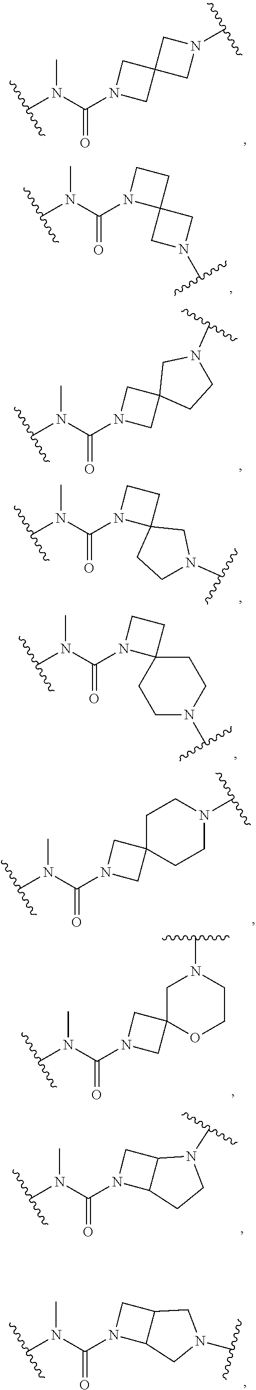

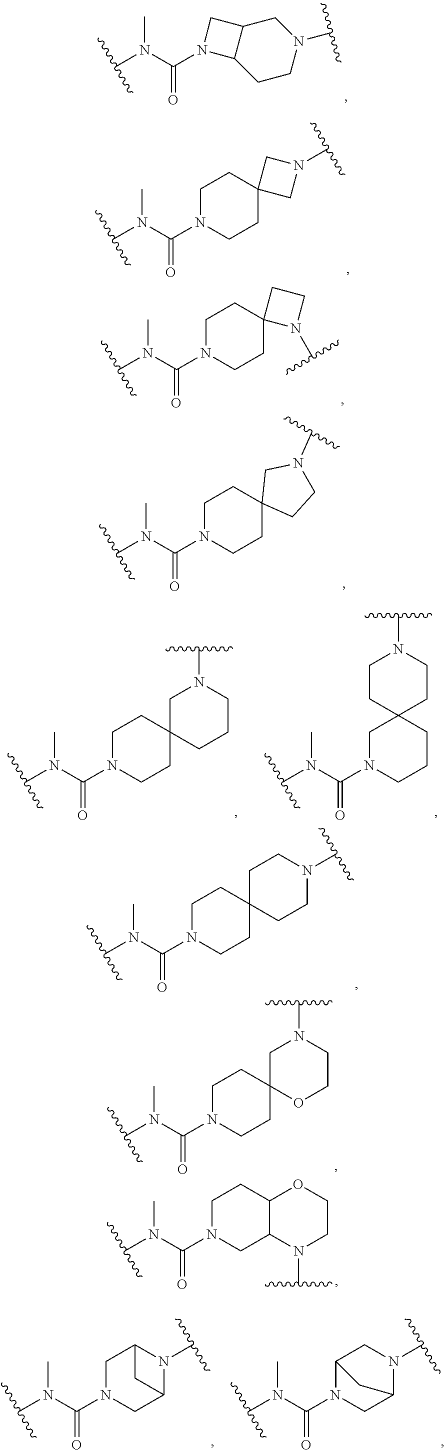

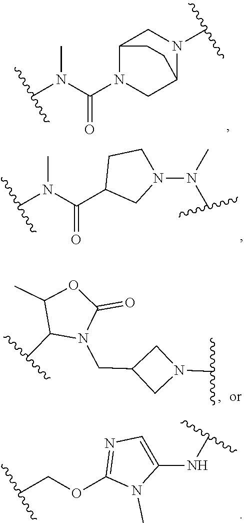

In some embodiments of Formula BI and subformula thereof, the linker has the structure

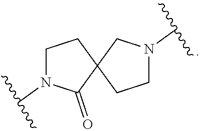 [SMILES: *N1CCC2(CCN(*)C2=O)C1]

In some embodiments of Formula BI and subformula thereof, the linker has the structure

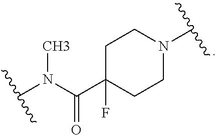 [SMILES: *N1CCC(F)(C(=O)N(*)C)CC1]

In some embodiments of Formula BI and subformula thereof, W is a cross-linking group comprising a vinyl ketone. In some embodiments, W has the structure of Formula BIIIa:

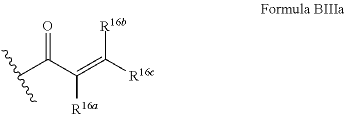

wherein R^{16a}, R^{16b}, and R^{16c} are, independently, hydrogen, —CN, halogen, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is:

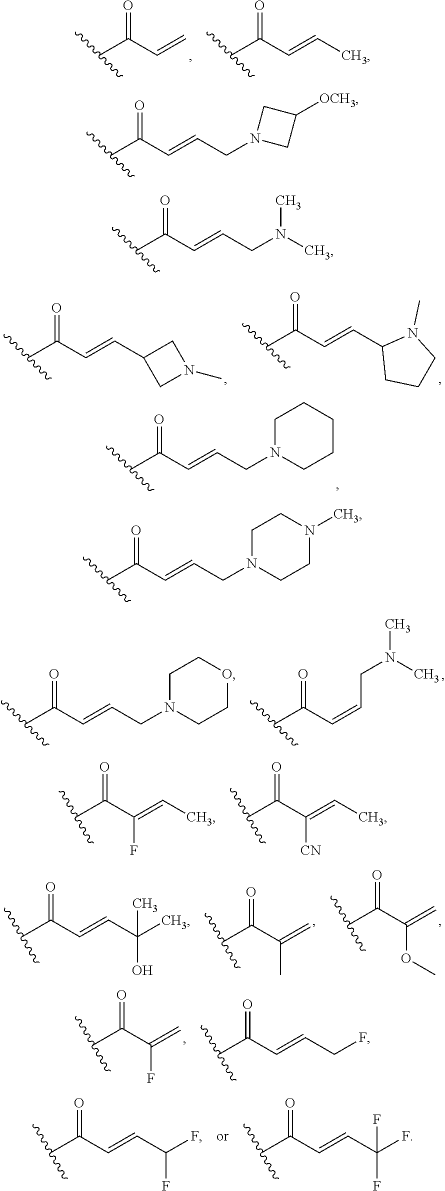

In some embodiments of Formula BI and subformula thereof, W is a cross-linking group comprising an ynone. In some embodiments, W has the structure of Formula BIIIb:

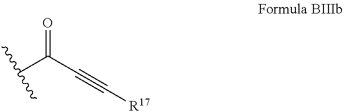

wherein R¹⁷ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl. In some embodiments, W is:

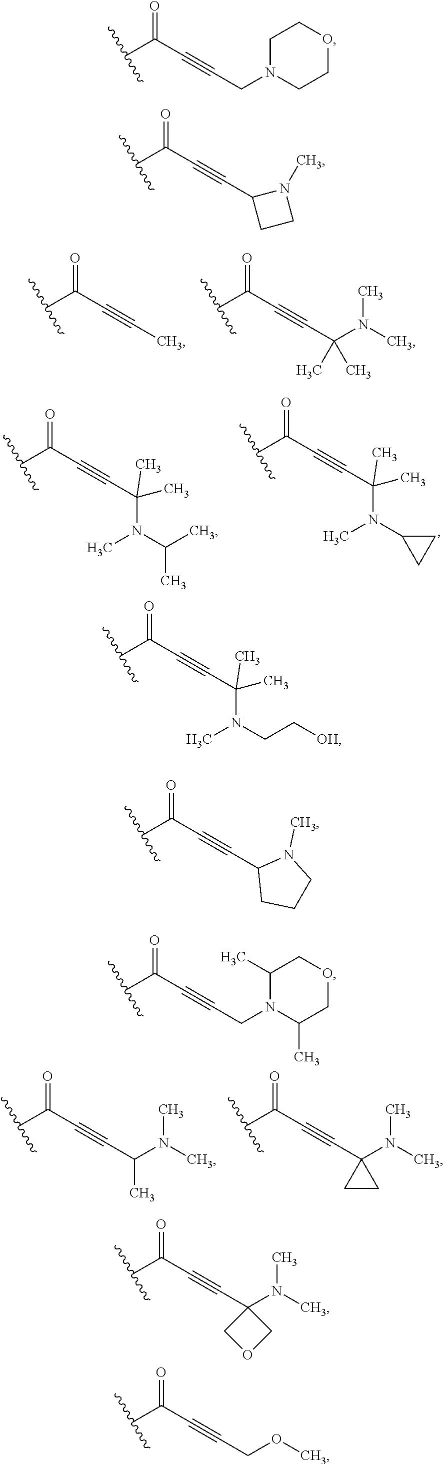

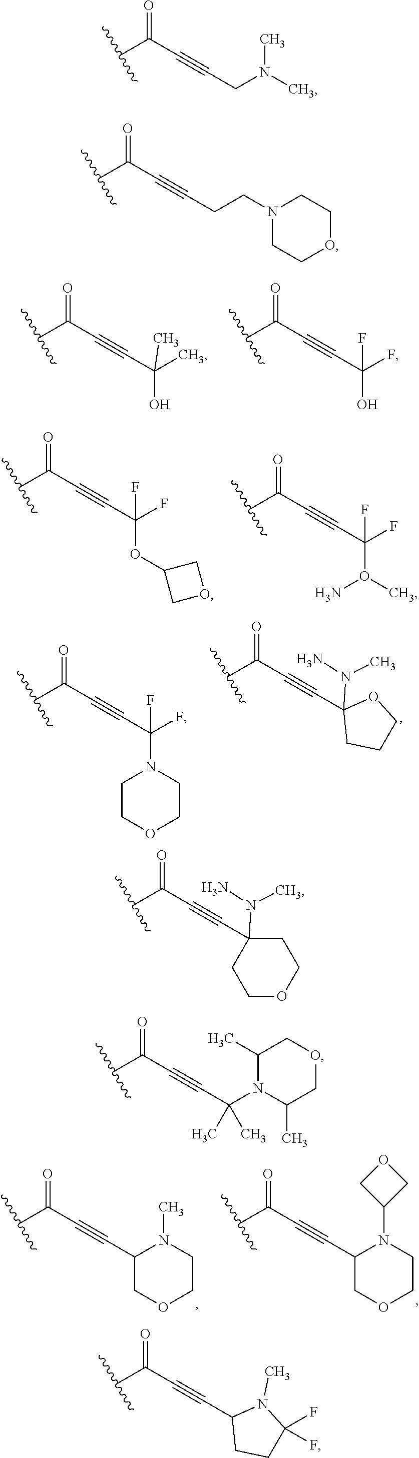

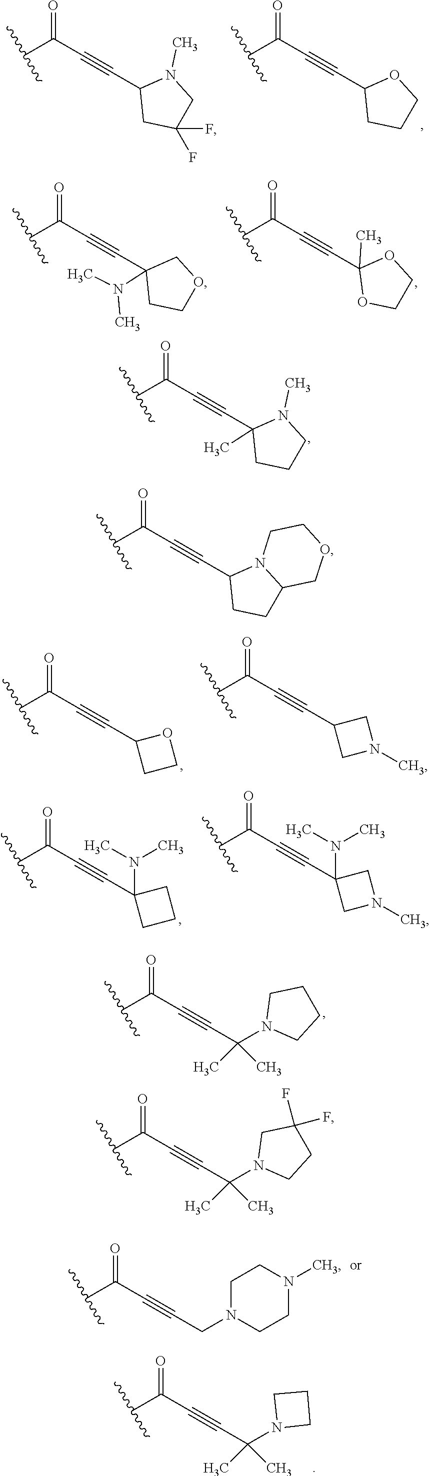

In some embodiments, W is

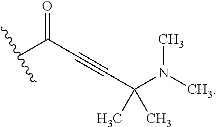 [SMILES: *C(=O)C#CC(C)(C)N(C)C]

In some embodiments of Formula BI and subformula thereof, W is a cross-linking group comprising a vinyl sulfone. In some embodiments, W has the structure of Formula BIIIc:

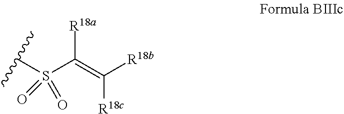

wherein R^{18a}, R^{18b}, and R^{18c} are, independently, hydrogen, —CN, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is:

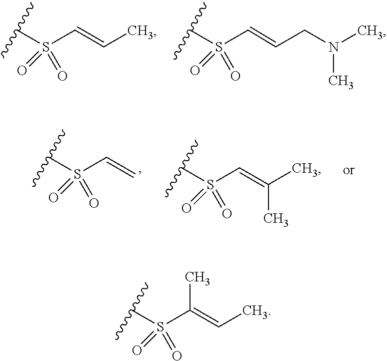

In some embodiments of Formula BI and subformula thereof, W is a cross-linking group comprising an alkynyl sulfone. In some embodiments, W has the structure of Formula BIIId:

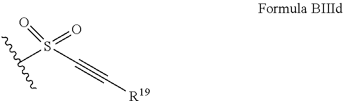

wherein R¹⁹ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl, or a 4 to 7-membered saturated heterocycloalkyl. In some embodiments, W is:

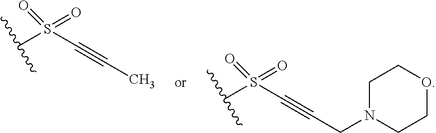

In some embodiments of Formula BI and subformula thereof, W has the structure of Formula BIIIe:

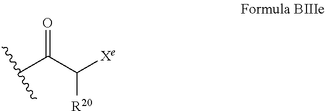

wherein X^{e} is a halogen; and
R²⁰ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments of Formula BI and subformula thereof, W is haloacetyl. In some embodiments of Formula BI and subformula thereof, W is not haloacetyl.

In some embodiments, the RAS(ON) inhibitor is selected from Table B1, or a pharmaceutically acceptable salt or stereoisomer thereof. In some embodiments, the RAS(ON) inhibitor is selected from Table B1, or a pharmaceutically acceptable salt or atropisomer thereof.

TABLE B1
Certain Compounds of the Present Invention

| Ex# | Structure |
|---|---|
| BA 1 | 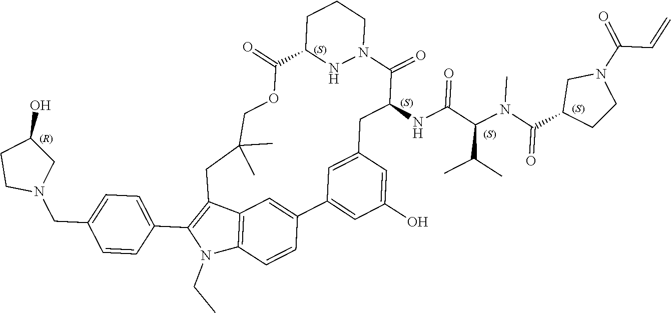 |
| BA 2 | 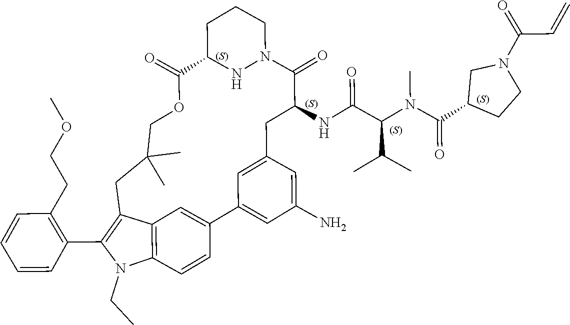 |
| BA 3 | 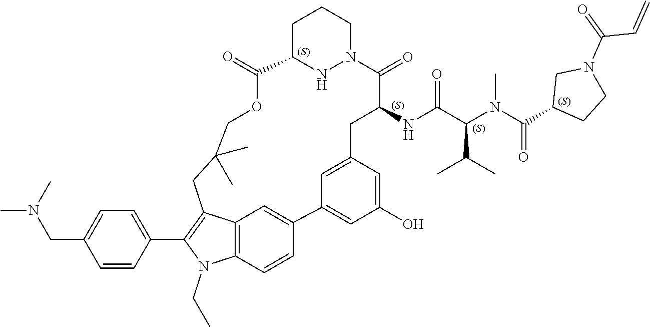 |
| BA 4 | 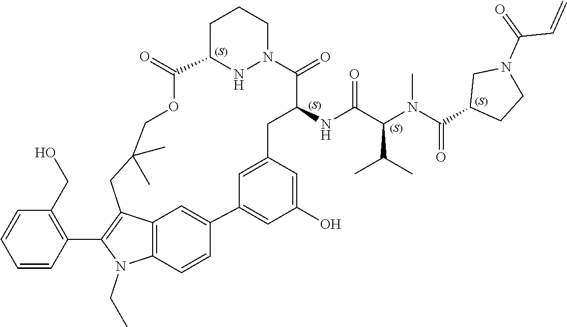 |
| BA 5 | 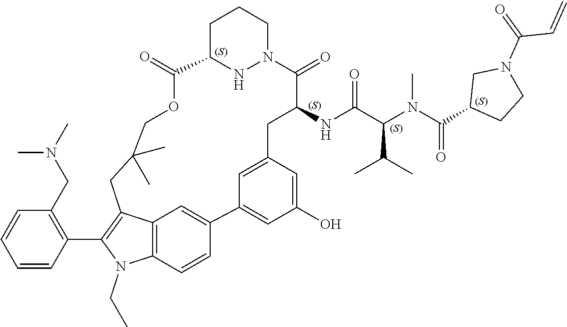 |
| BA 6 | 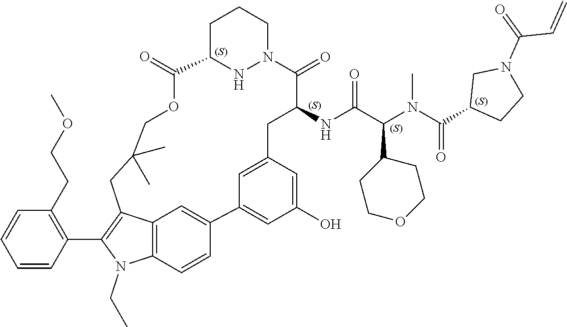 |
| BA 7 | 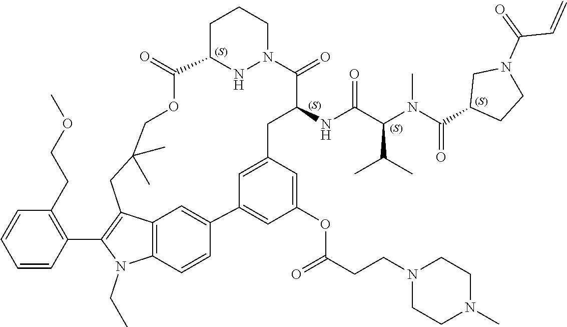 |
| BA 8 | 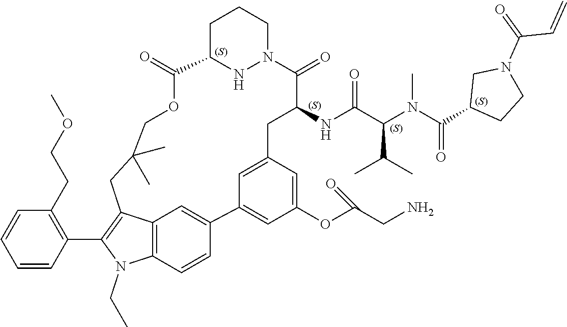 |
| BA 9 | 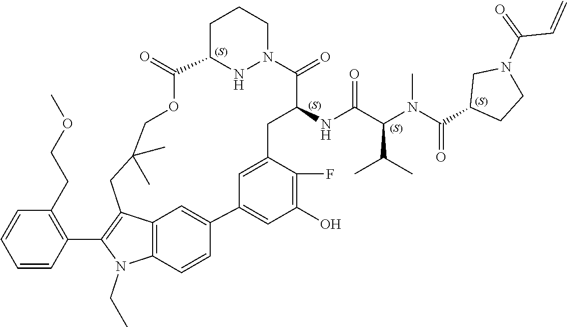 |
| BA 10 | 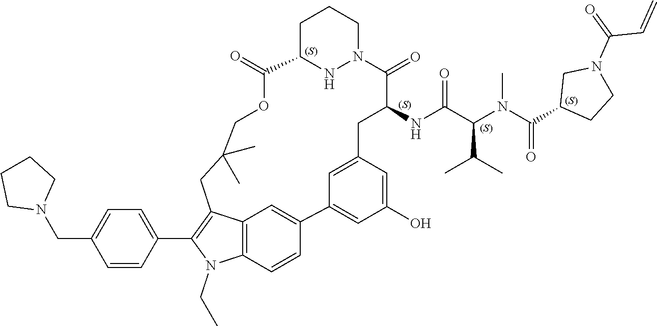 |
| BA 11 | 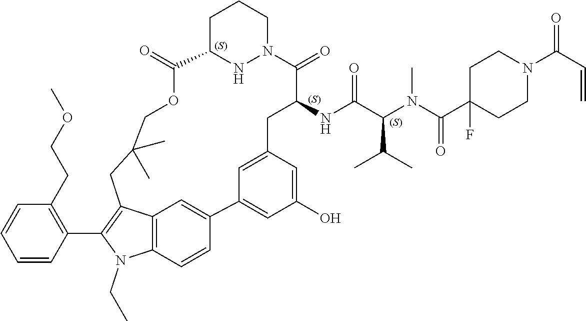 |
| BA 12 | 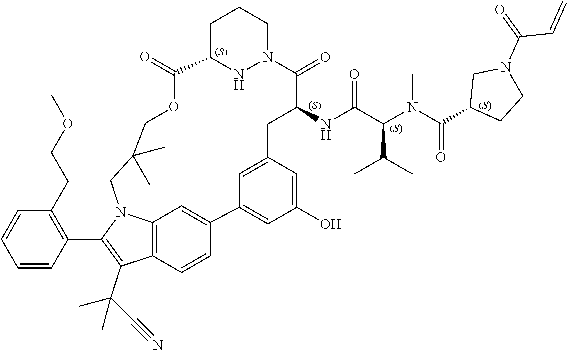 |
| BA 13 | 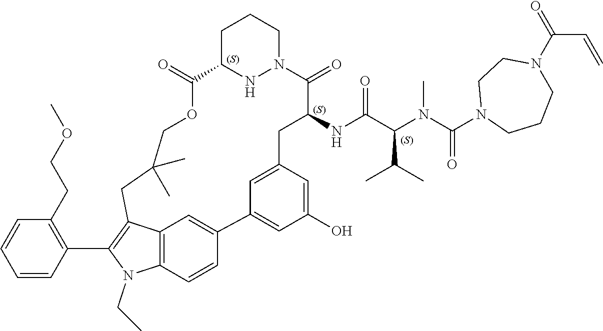 |
| BA 14 | 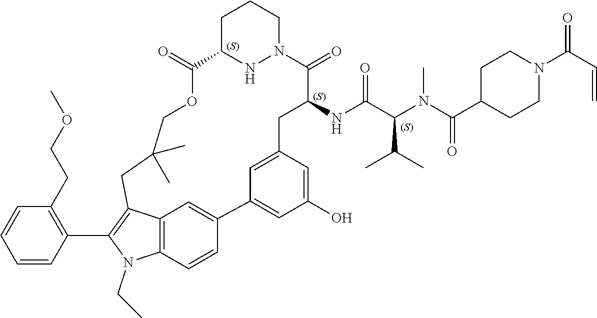 |
| BA 15 | 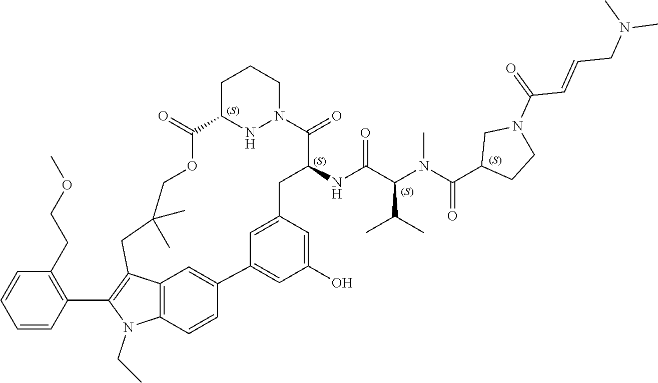 |
| BA 16 | 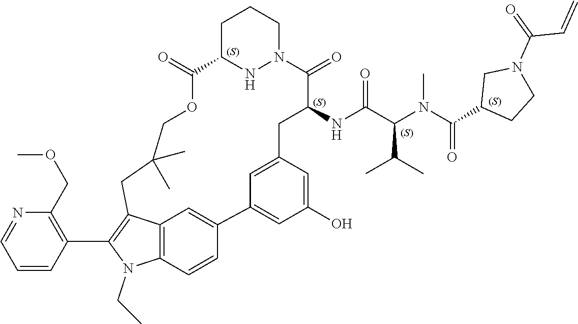 |
| BA 17 | 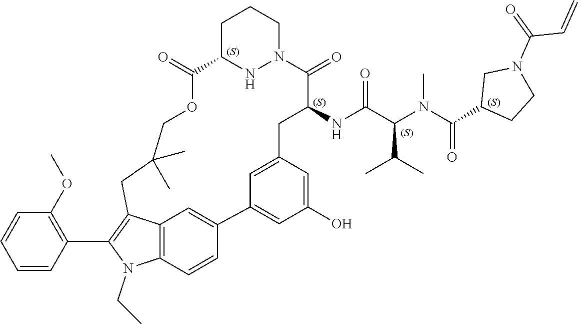 |
| BA 18 | 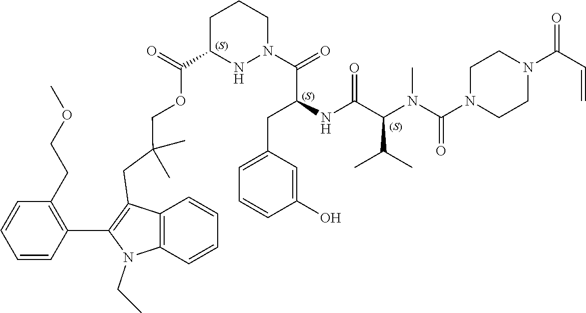 |
| BA 19 | 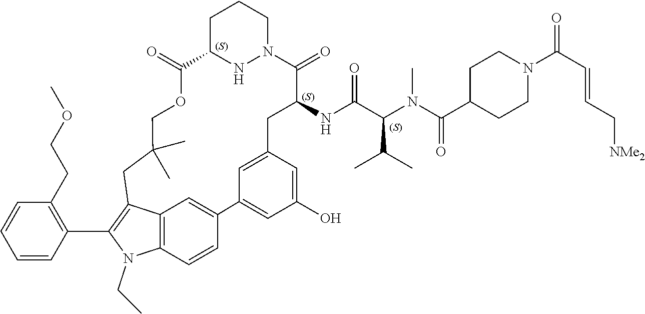 |
| BA 20 | 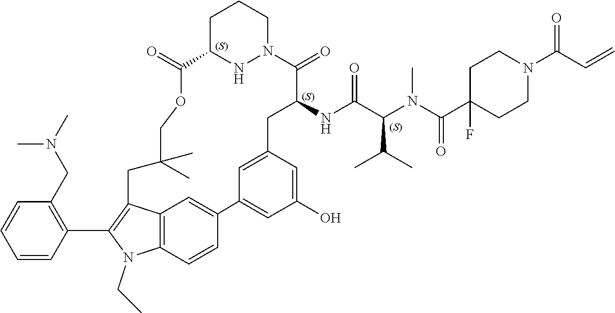 |
| BA 21 | 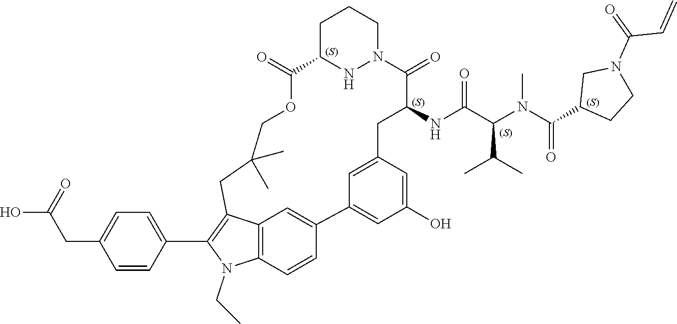 |
| BA 22 | 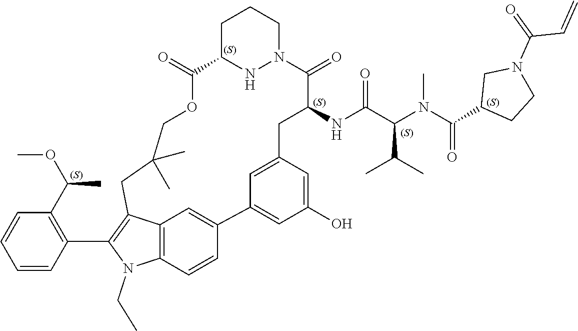 |
| BA 23 | 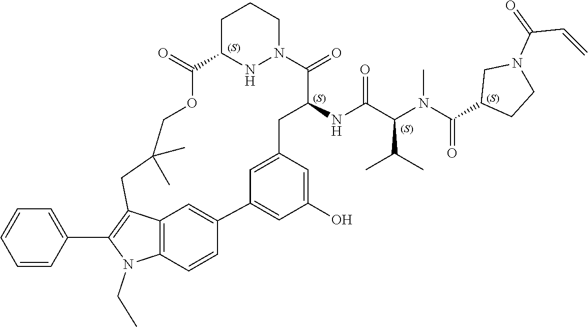 |
| BA 24 | 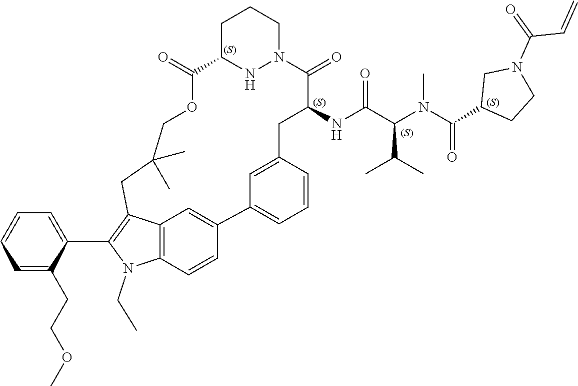 |
| BA 25 | 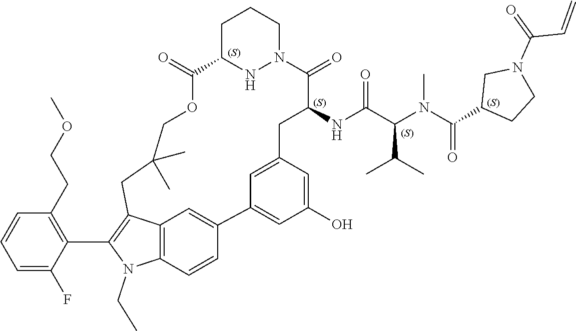 |
| BA 26 | 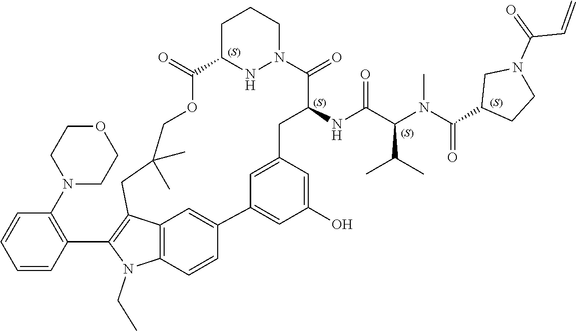 |
| BA 27 | 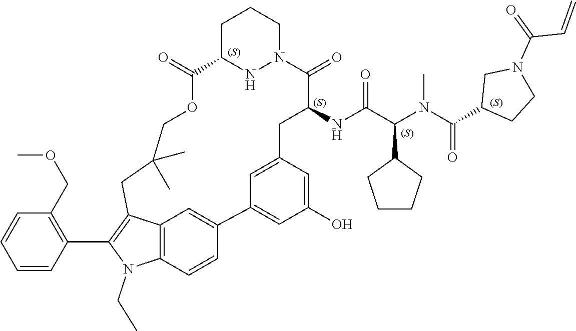 |
| BA 28 | 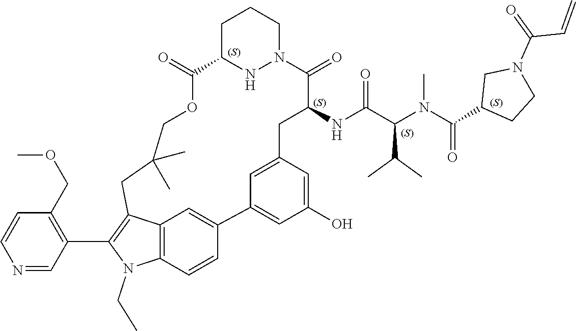 |
| BA 29 | 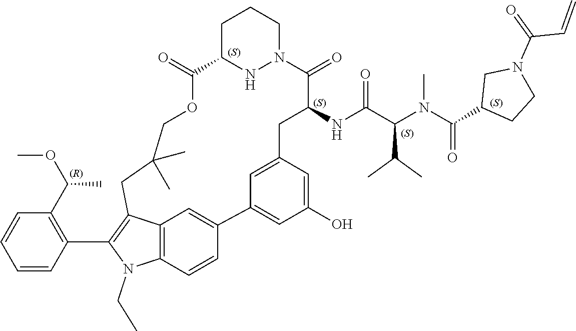 |
| BA 30 | 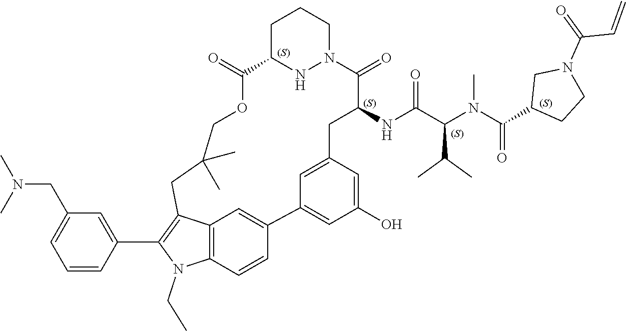 |
| BA 31 | 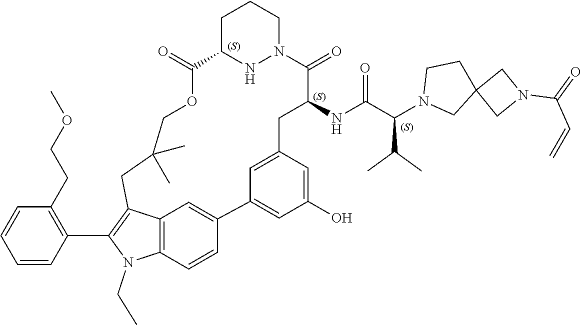 |
| BA 32 | 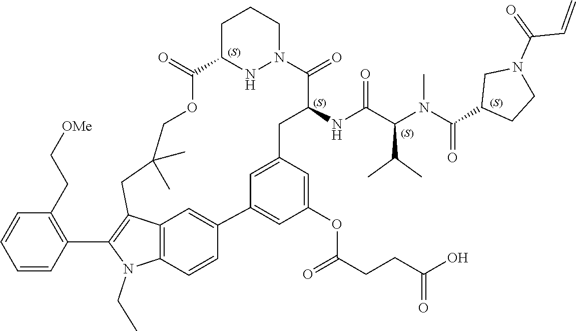 |
| BA 33 | 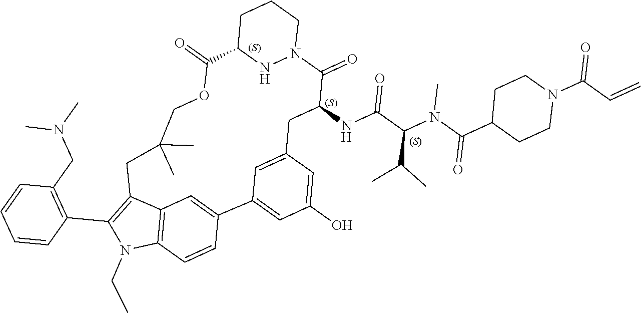 |
| BA 34 | 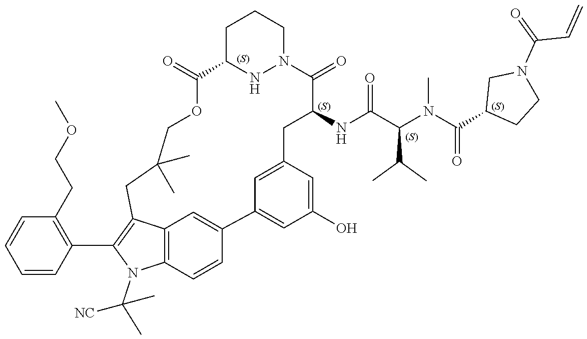 |
| BA 35 | 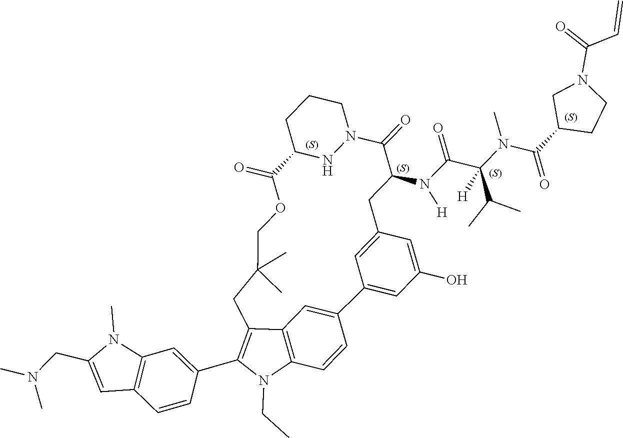 |
| BA 36 | 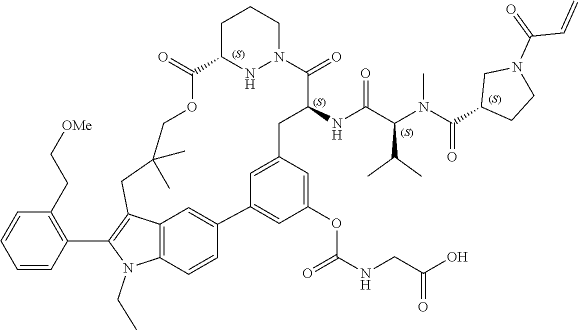 |
| BA 37 | 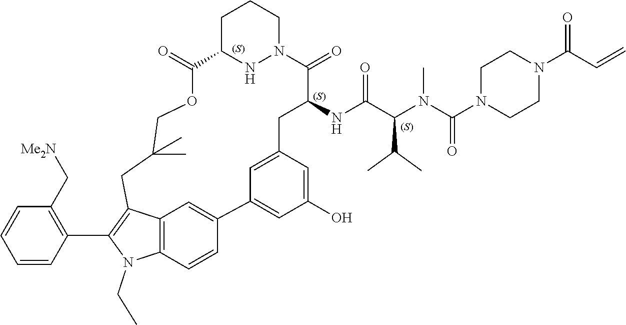 |
| BA 38 | 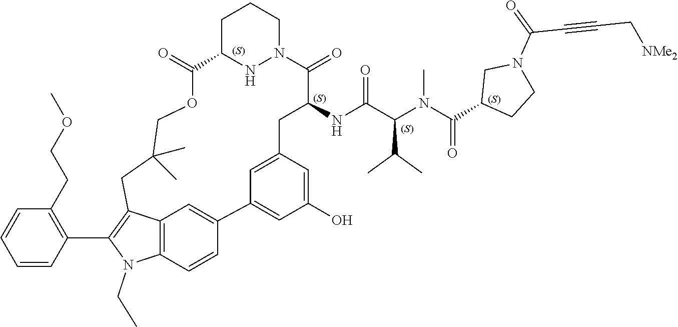 |
| BA 39 | 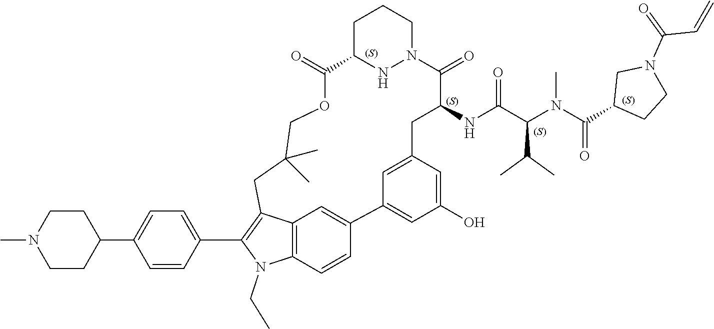 |
| BA 40 | 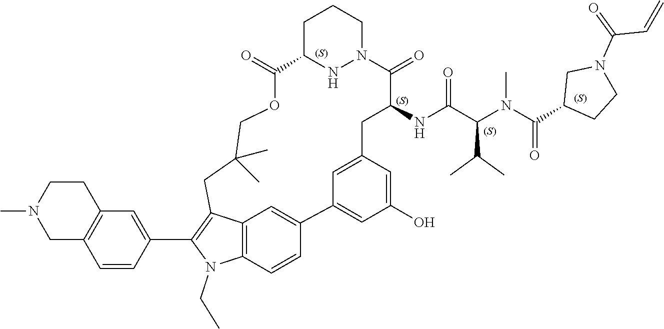 |
| BA 41 | 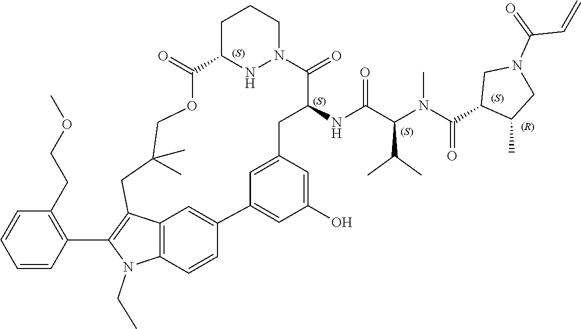 |
| BA 42 | 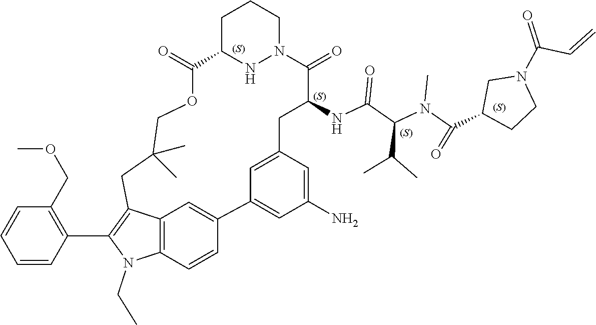 |
| BA 43 | 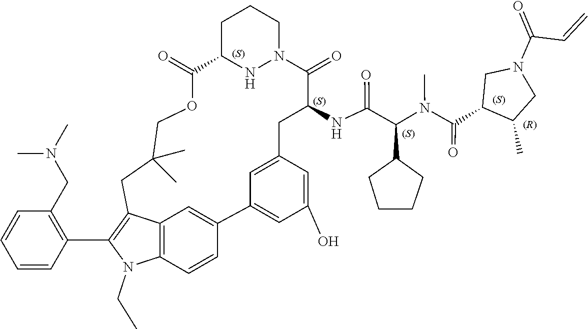 |
| BA 44 | 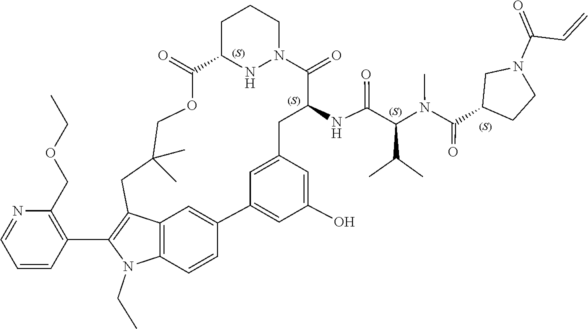 |
| BA 45 | 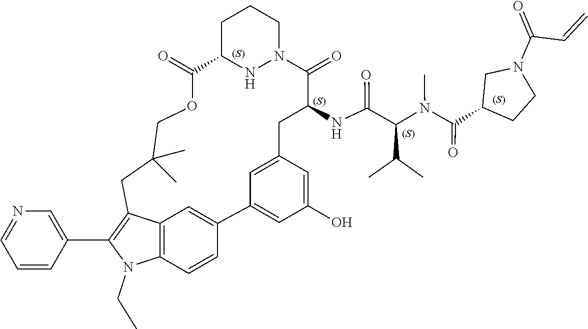 |
| BA 46 | 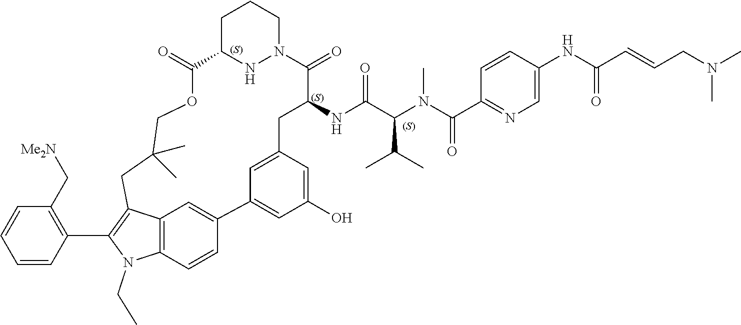 |
| BA 47 | 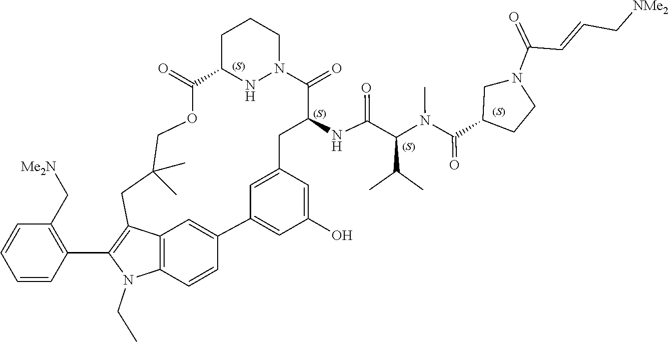 |
| BA 48 | 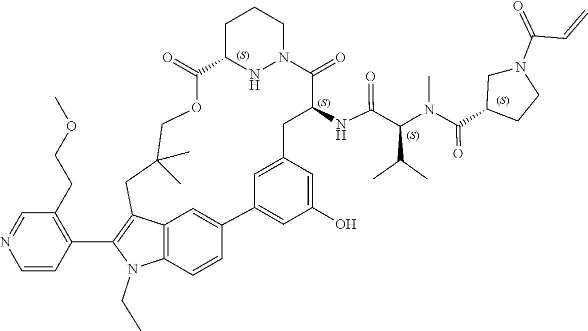 |
| BA 49 | 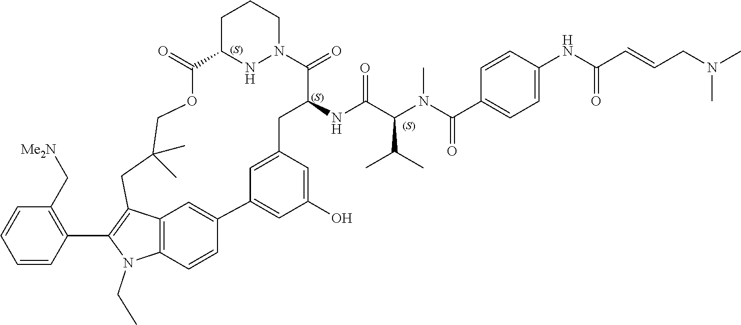 |
| BA 50 | 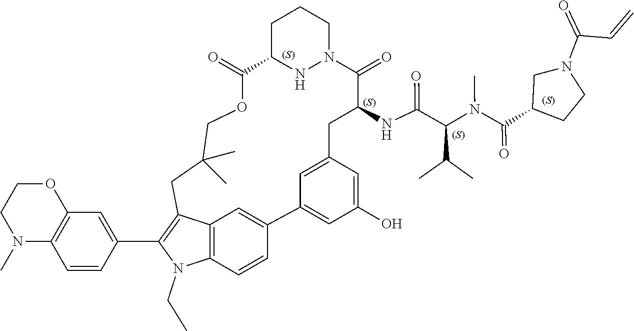 |
| BA 51 | 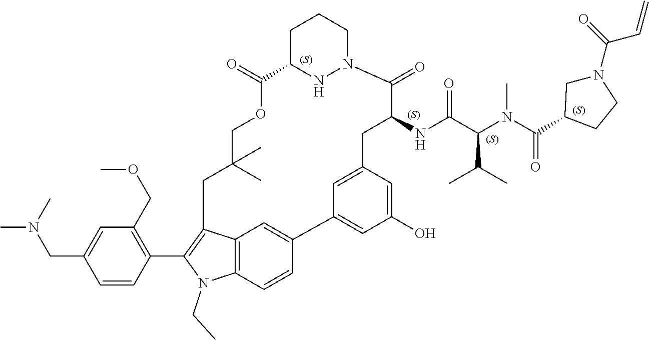 |
| BA 52 | 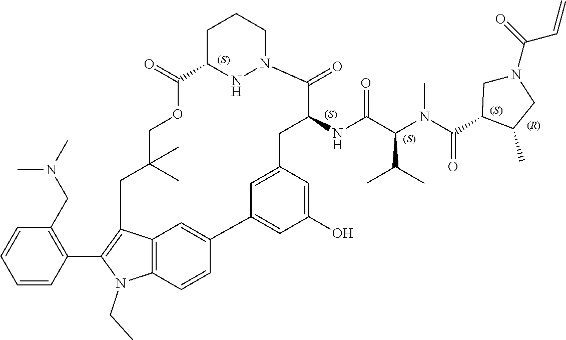 |
| BA 53 | 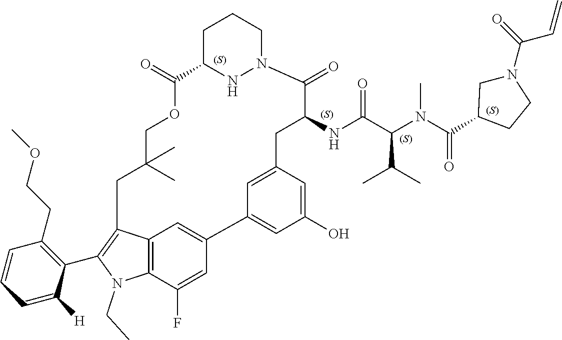 |
| BA 54 | 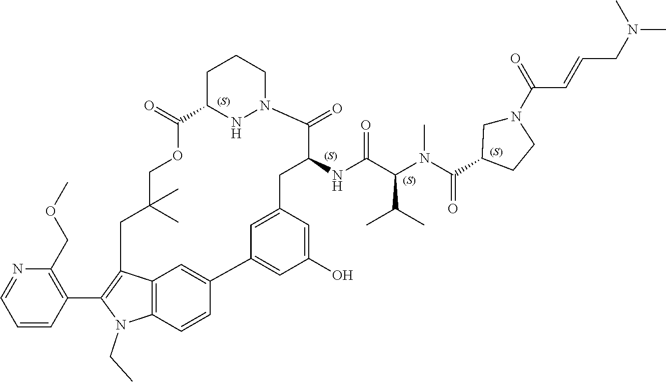 |
| BA 55 | 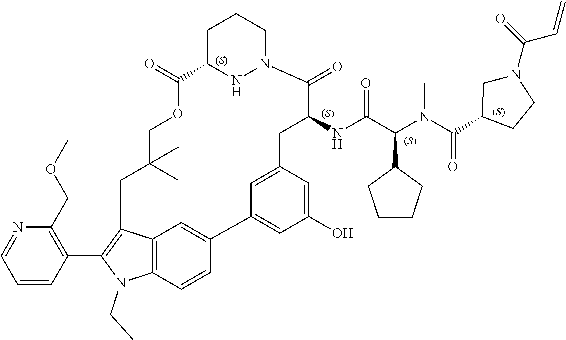 |
| BA 56 | 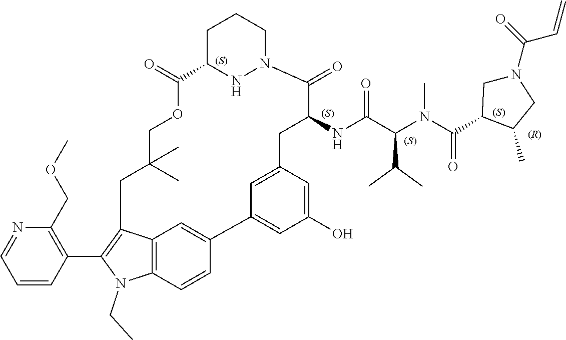 |
| BA 57 | 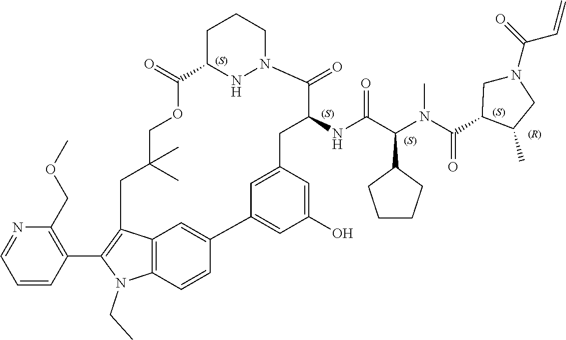 |
| BA 58 | 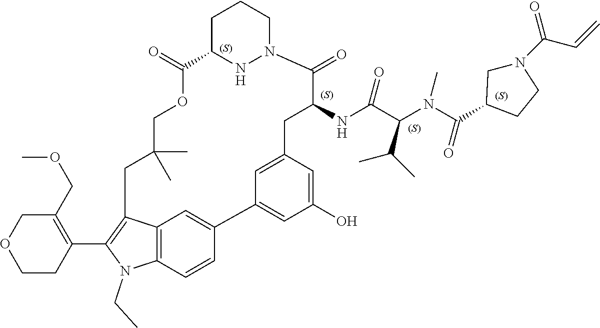 |
| BA 59 | 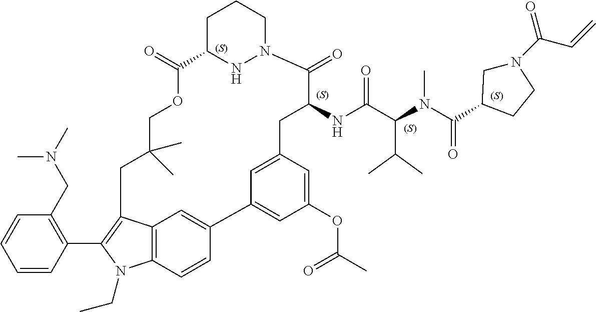 |
| BA 60 | 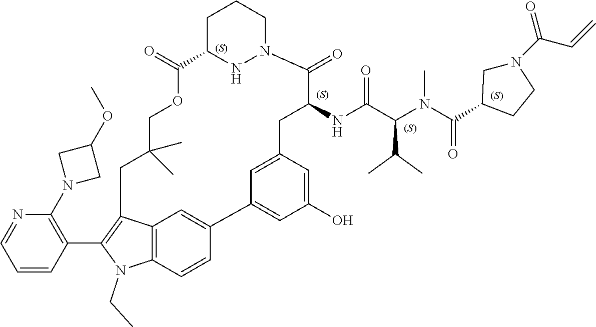 |
| BA 61 | 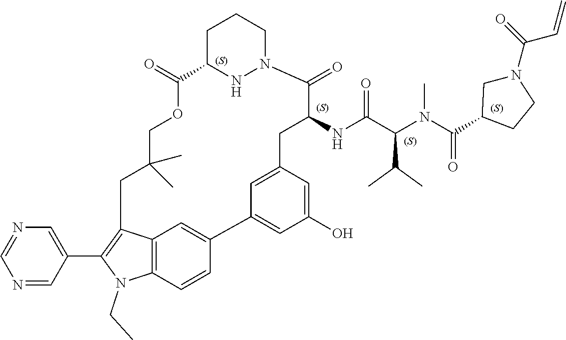 |
| BA 62 | 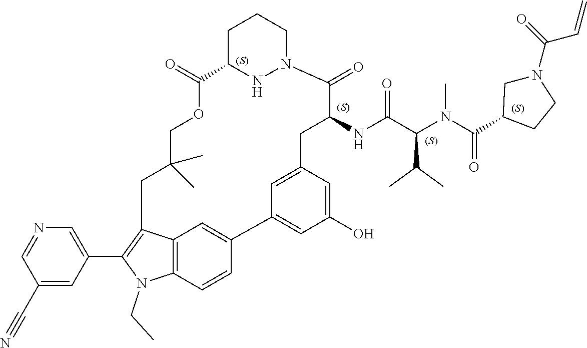 |
| BA 63 | 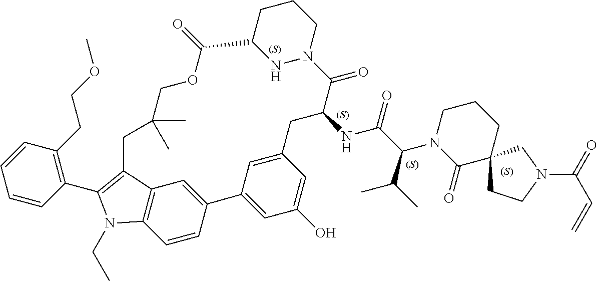 |
| BA 64 | 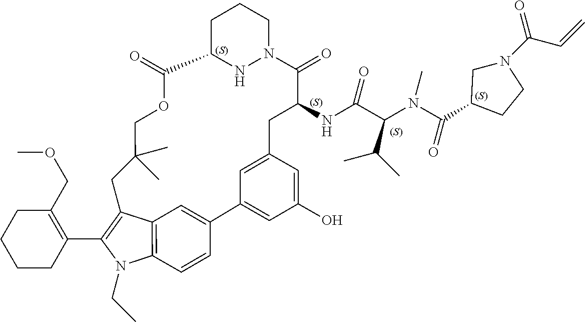 |
| BA 65 | 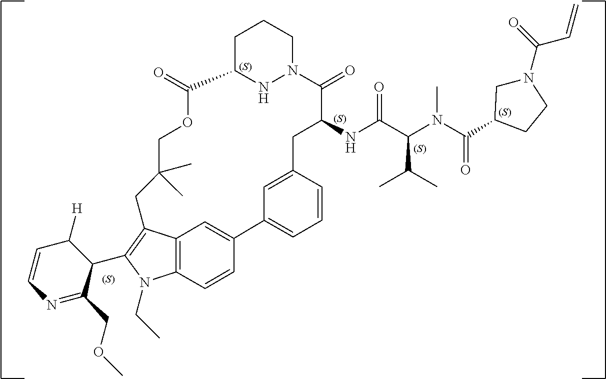 |
| BA 66 | 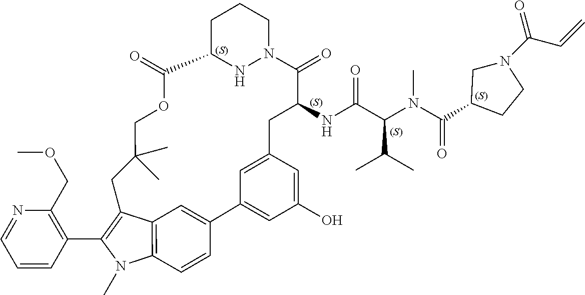 |
| BA 67 | 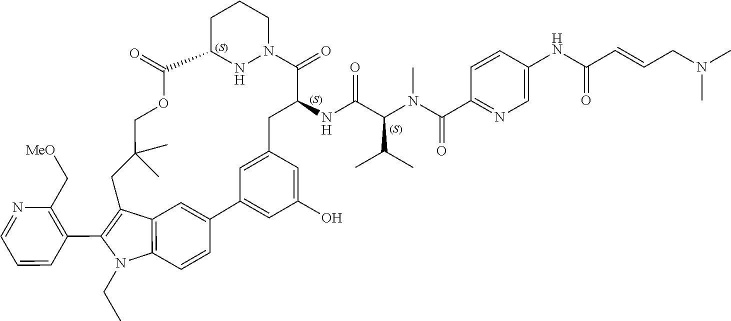 |
| BA 68 | 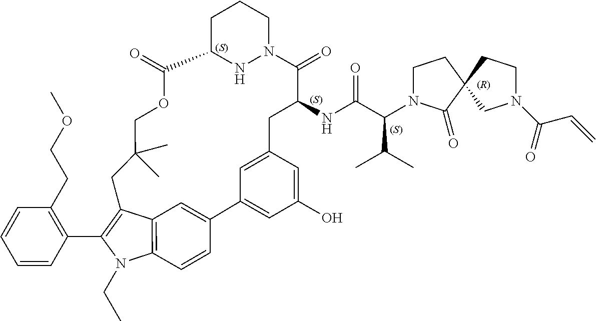 |
| BA 69 | 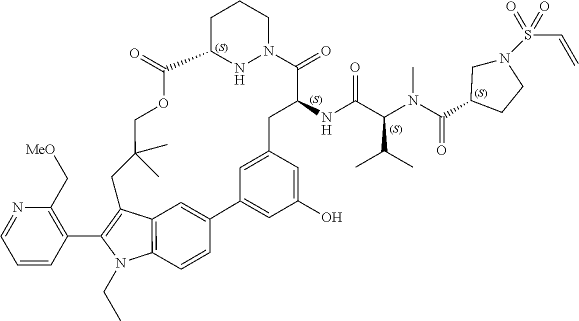 |
| BA 70 | 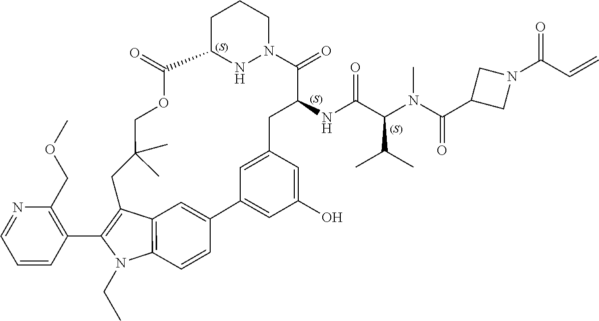 |
| BA 71 | 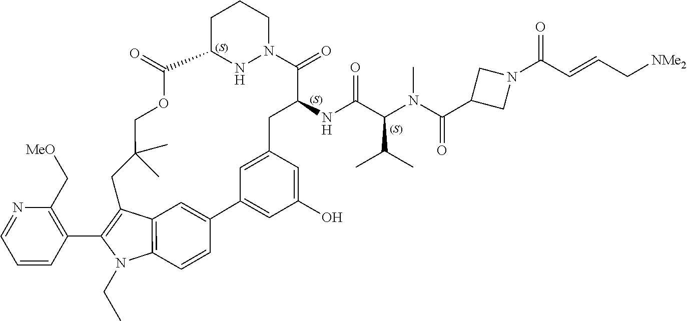 |
| BA 72 | 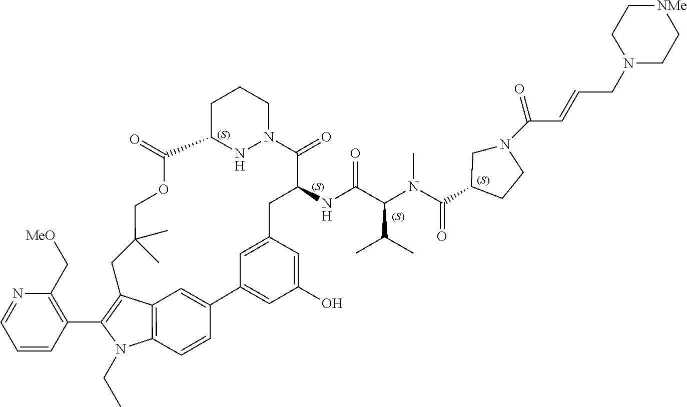 |
| BA 73 | 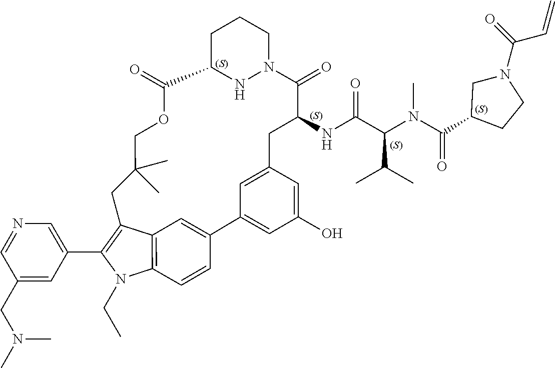 |
| BA 74 | 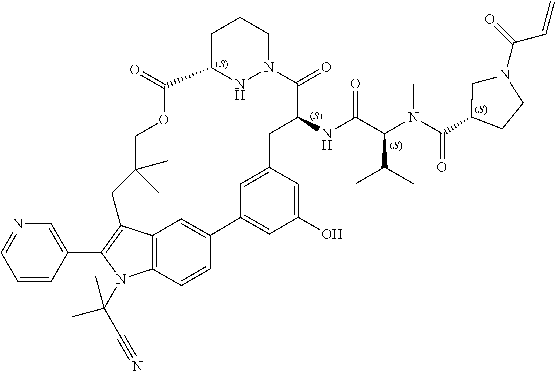 |
| BA 75 | 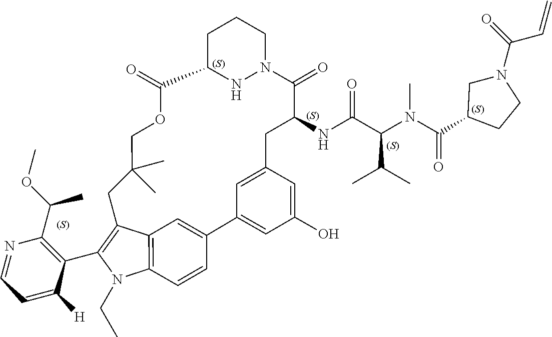 |
| BA 76 | 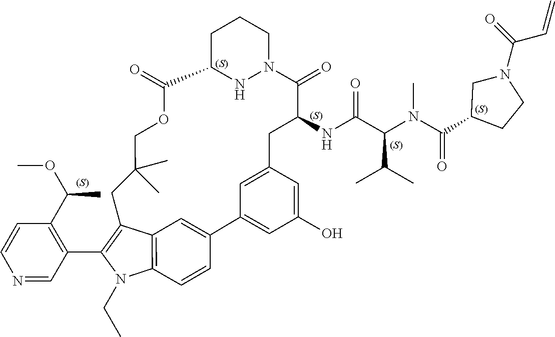 |
| BA 77 | 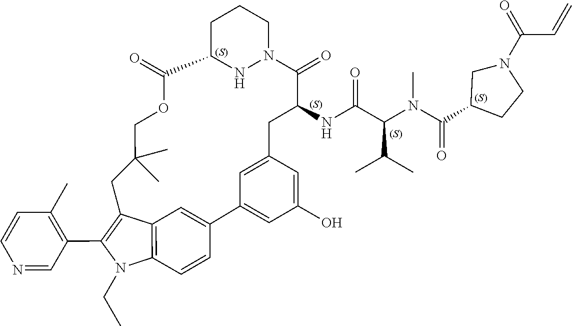 |
| BA 78 | 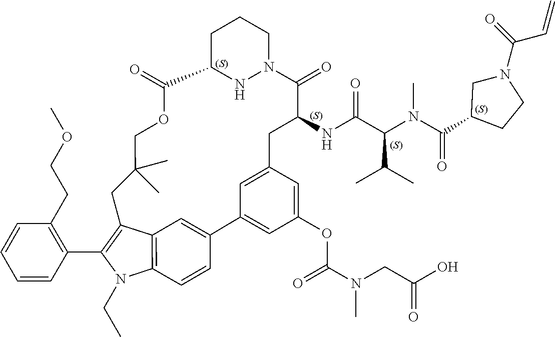 |
| BA 79 | 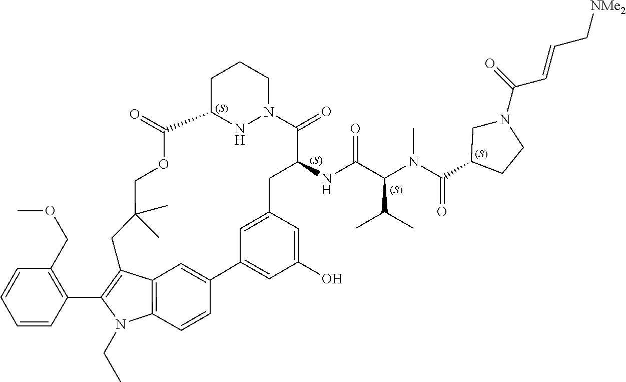 |
| BA 80 | 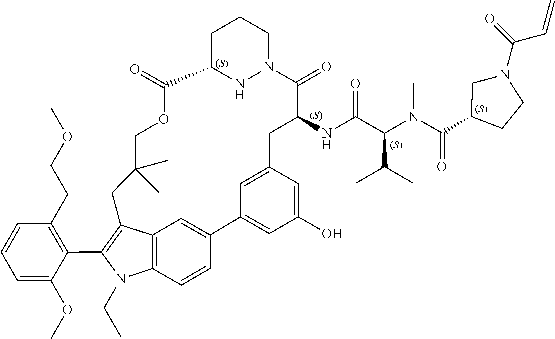 |
| BA 81 | 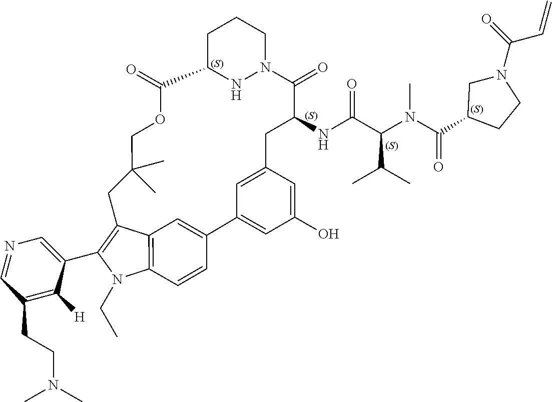 |
| BA 82 | 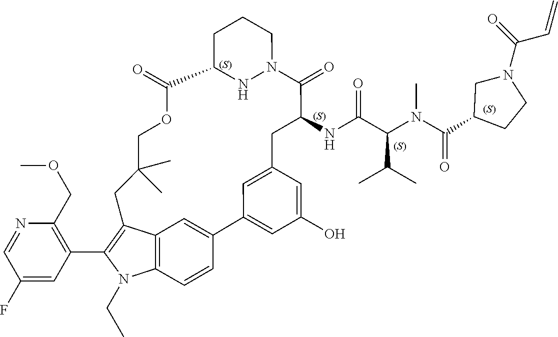 |
| BA 83 | 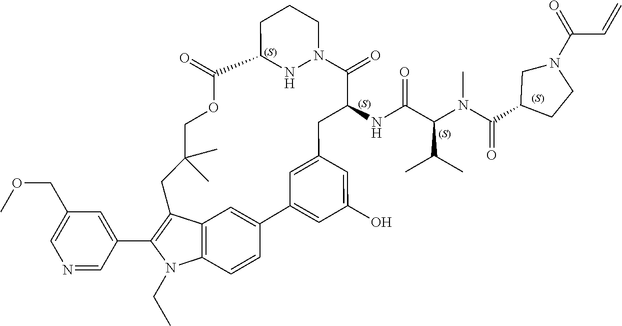 |
| BA 84 | 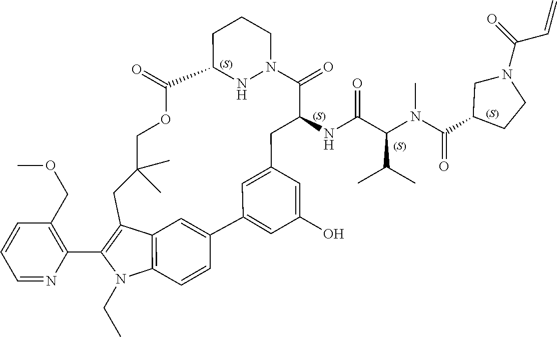 |
| BA 85 | 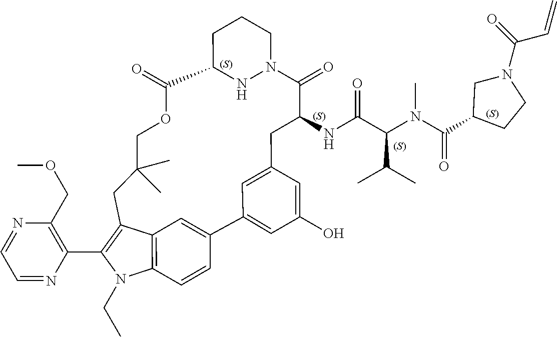 |
| BA 86 | 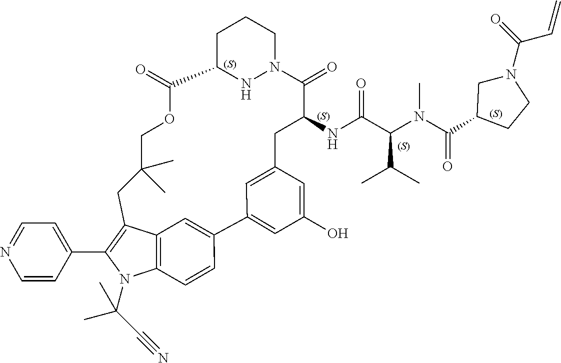 |
| BA 87 | 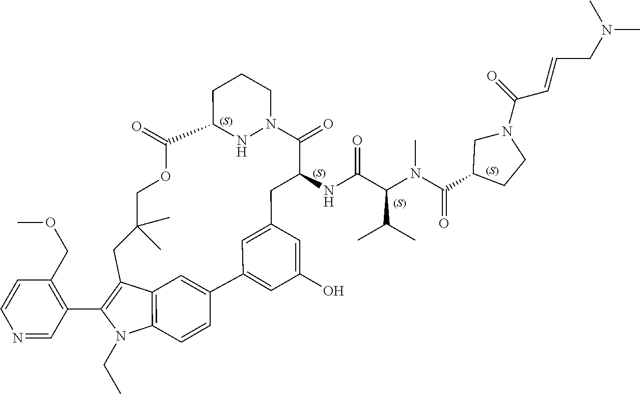 |
| BA 88 | 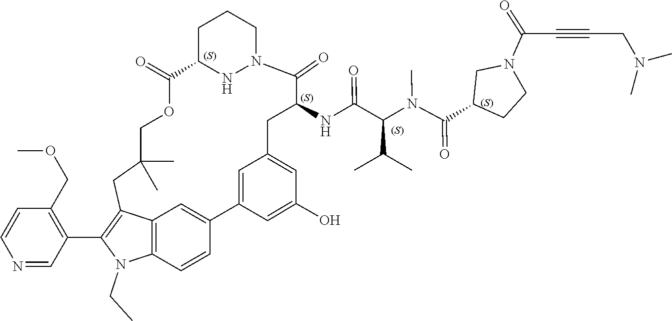 |
| BA 89 | 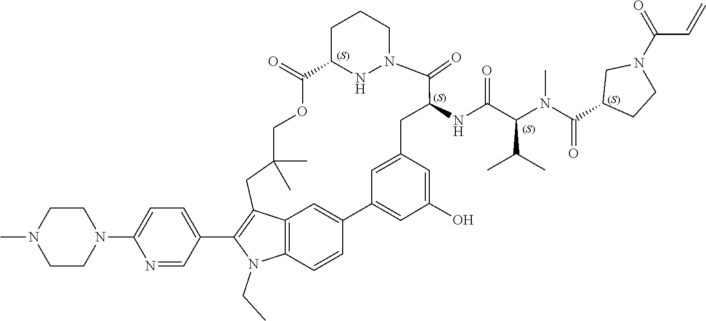 |
| BA 90 | 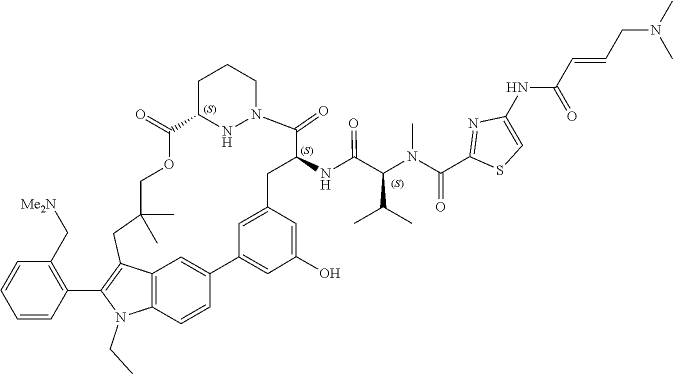 |
| BA 91 | 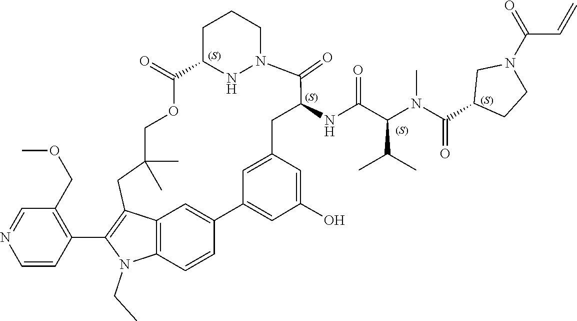 |
| BA 92 | 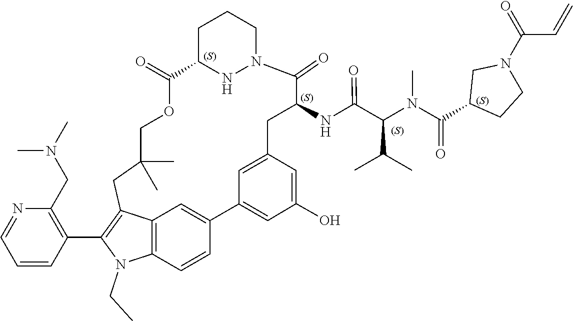 |
| BA 93 | 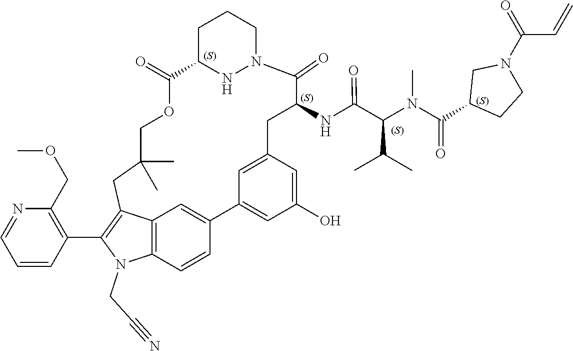 |
| BA 94 | 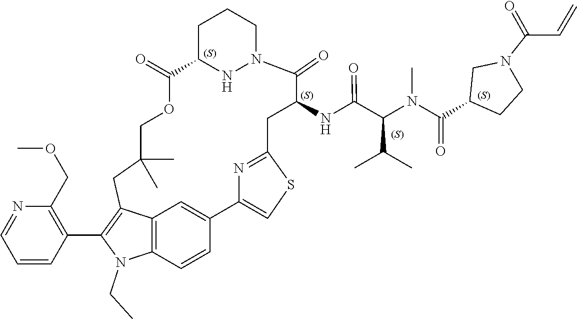 |
| BA 95 | 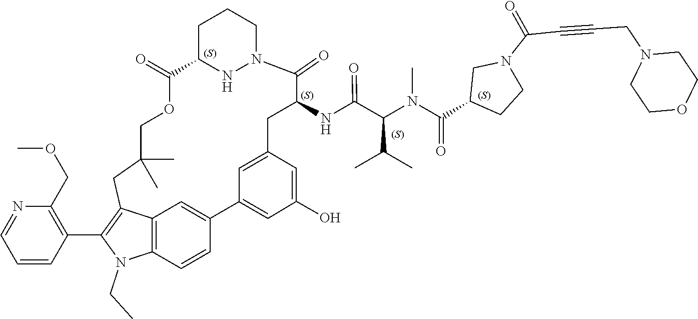 |
| BA 96 | 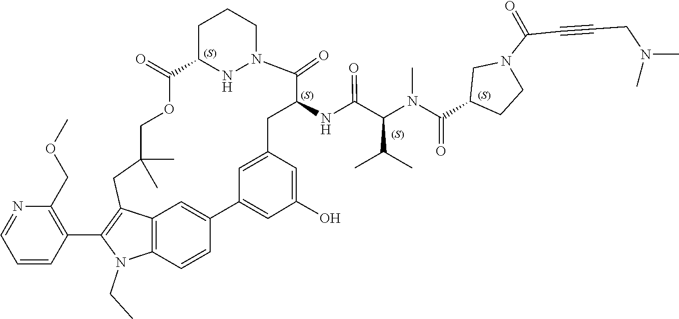 |
| BA 97 | 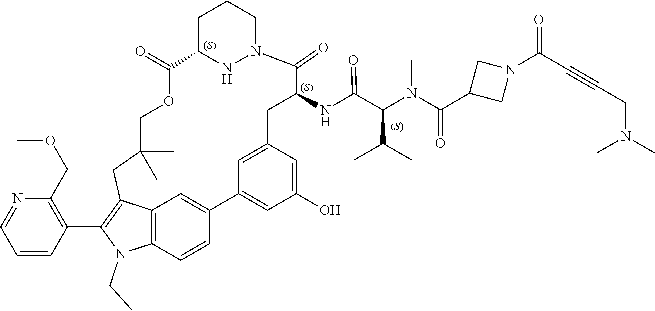 |
| BA 98 | 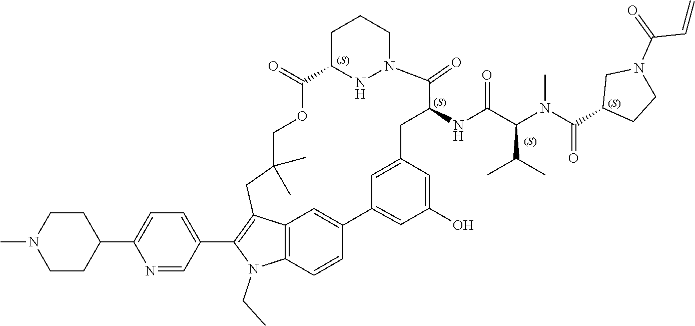 |
| BA 99 | 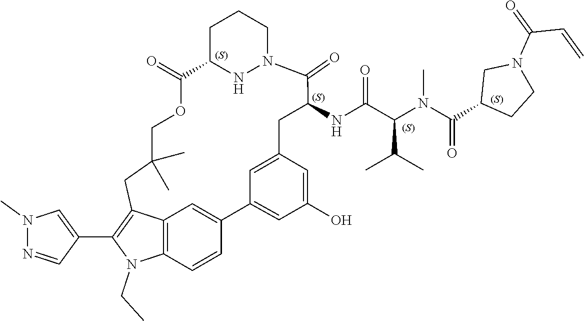 |
| BA 100 | 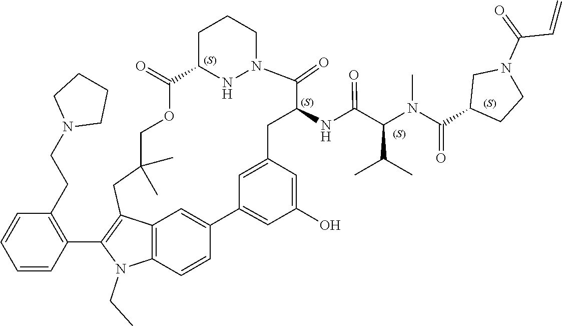 |
| BA 101 | 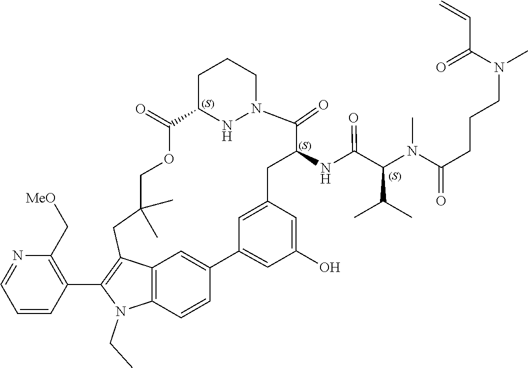 |
| BA 102 | 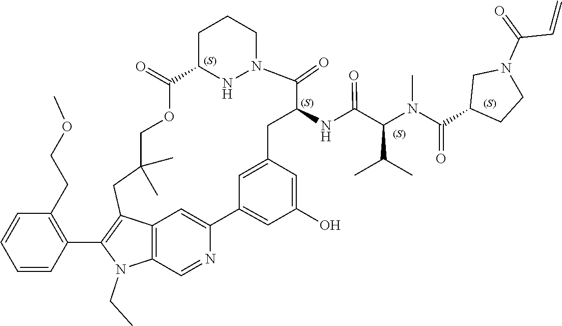 |
| BA 103 | 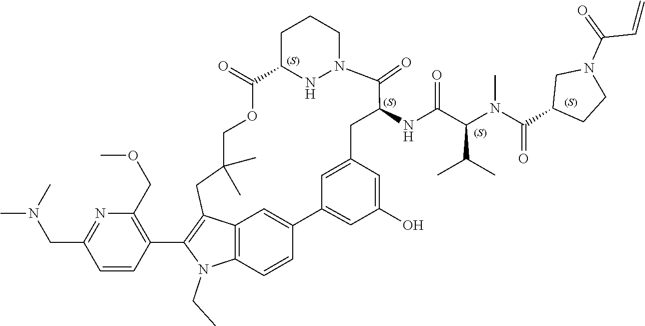 |
| BA 104 | 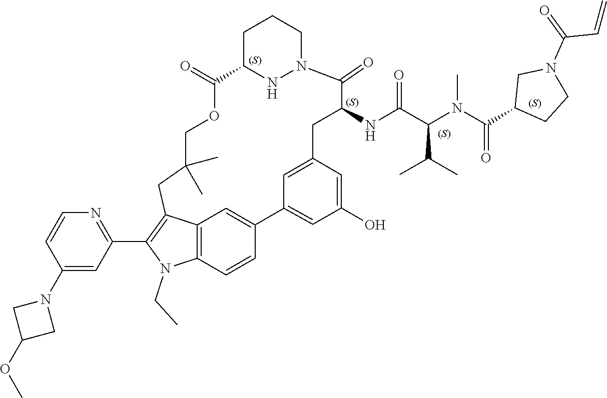 |
| BA 105 | 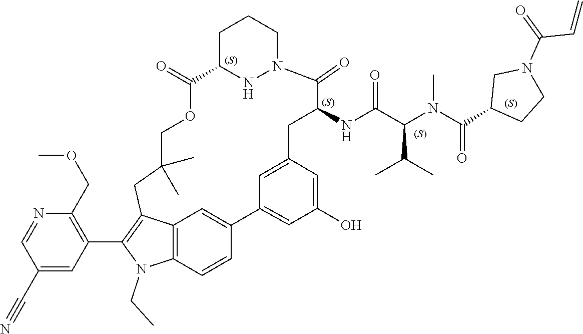 |
| BA 106 | 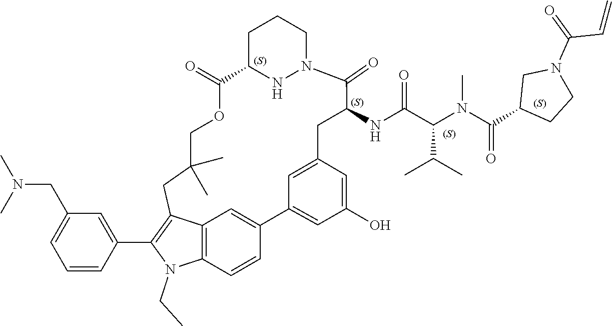 |
| BA 107 | 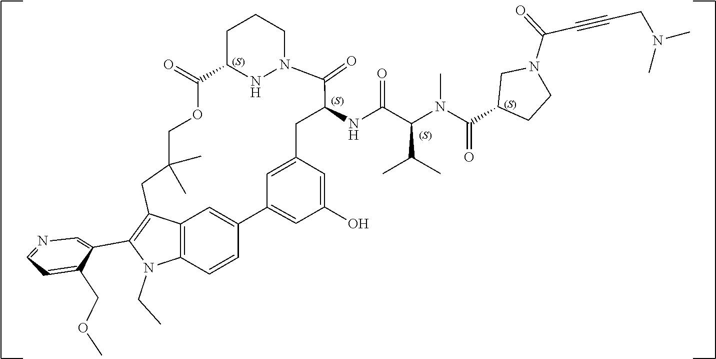 |
| BA 108 | 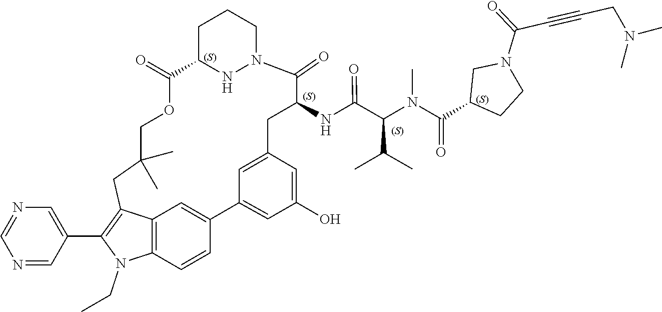 |
| BA 109 | 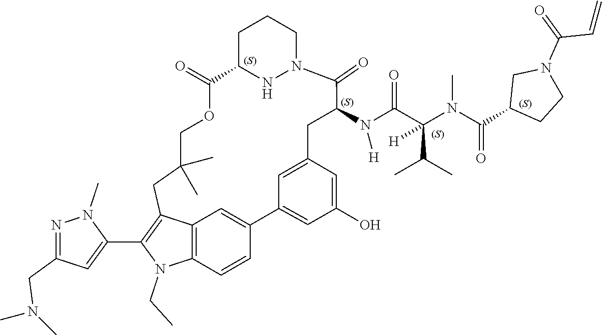 |
| BA 110 | 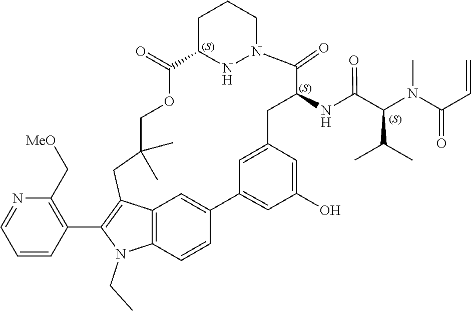 |
| BA 111 | 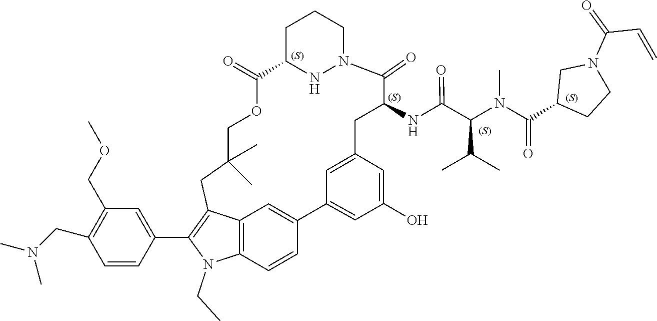 |
| BA 112 | 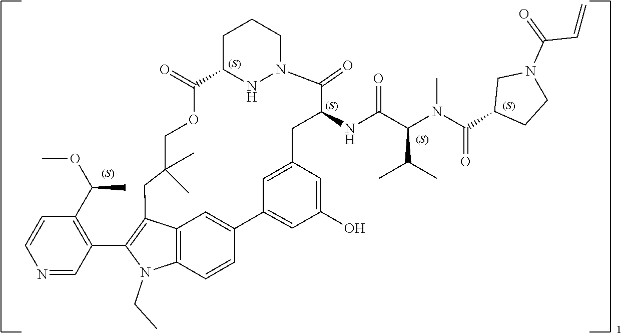 |
| BA 113 | 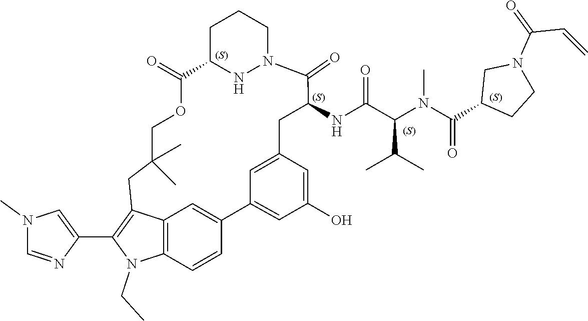 |
| BA 114 | 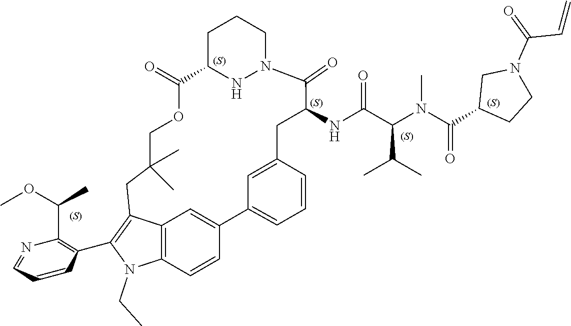 |
| BA 115 | 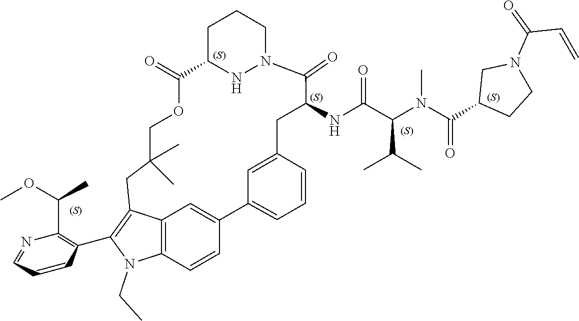 |
| BA 116 | 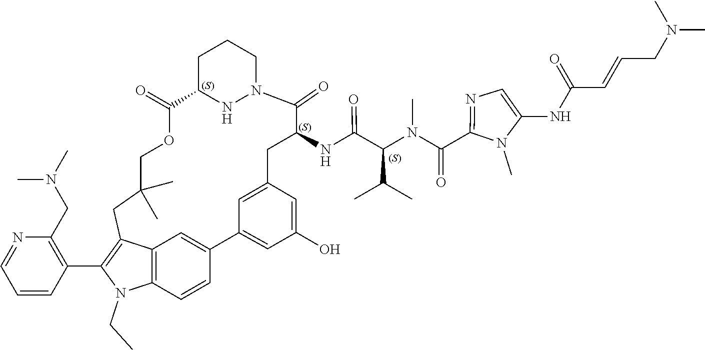 |
| BA 117 | 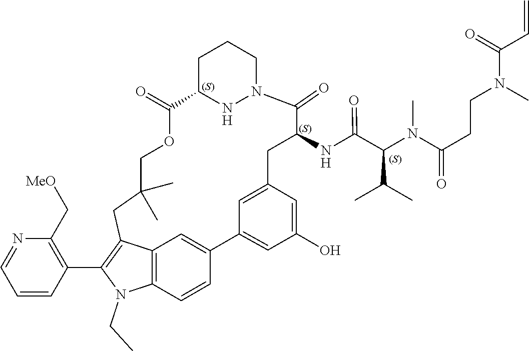 |
| BA 118 | 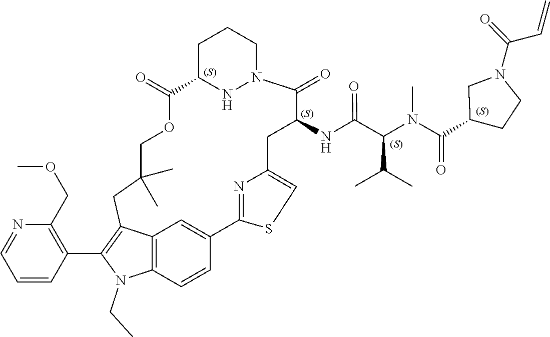 |
| BA 119 | 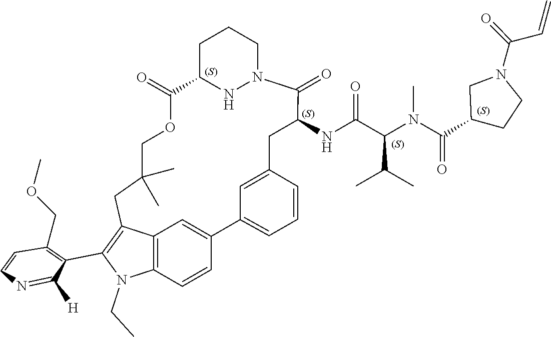 |
| BA 120 | 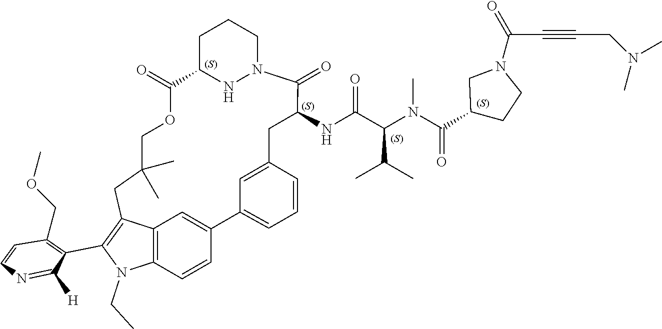 |
| BA 121 | 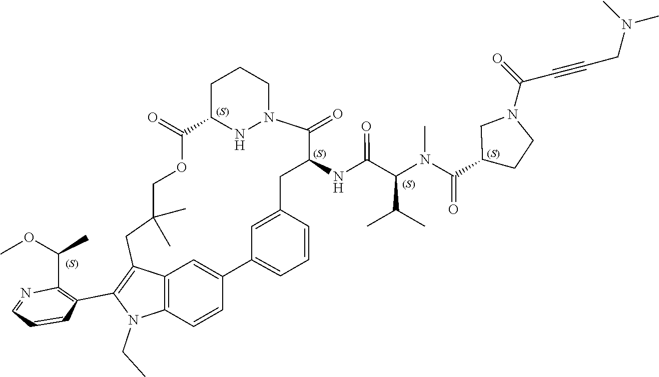 |
| BA 122 | 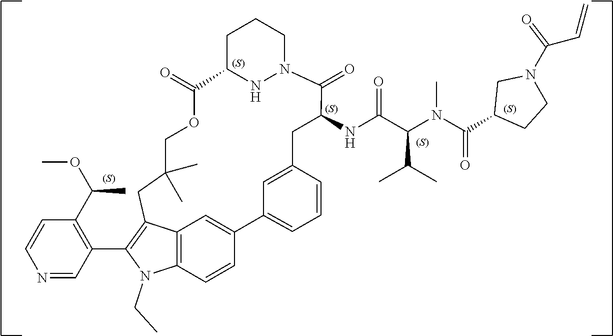 |
| BA 123 | 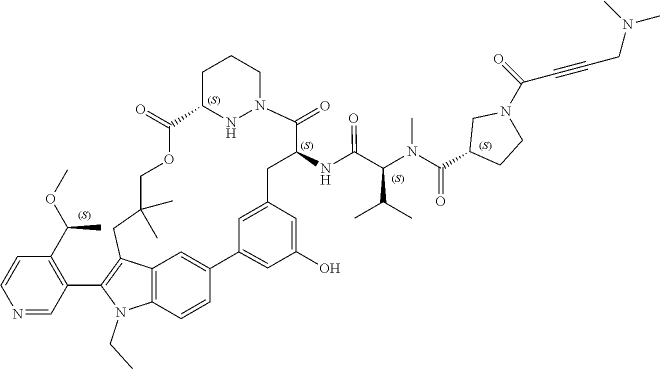 |
| BA 124 | 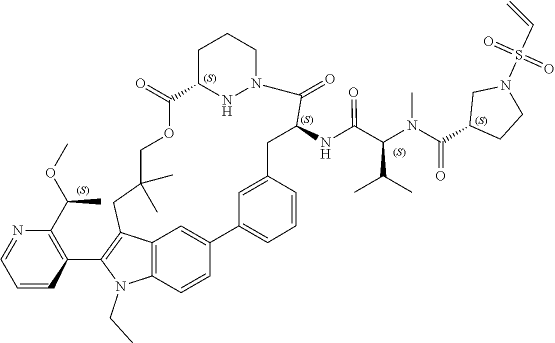 |
| BA 125 | 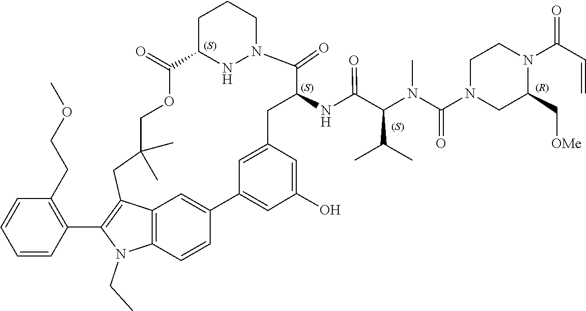 |
| BA 126 | 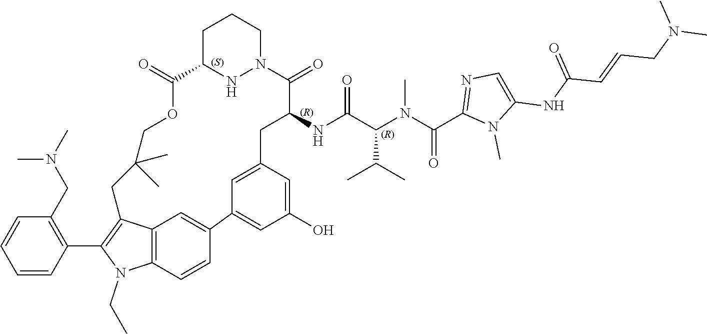 |
| BA 127 | 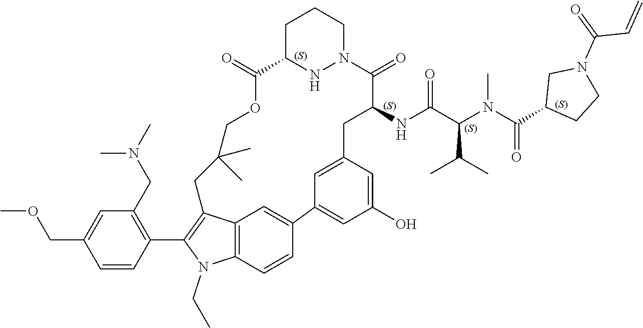 |
| BA 128 | 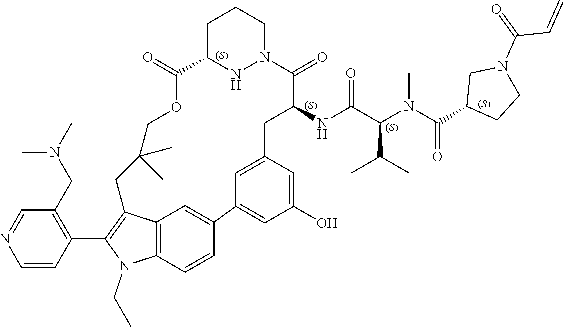 |
| BA 129 | 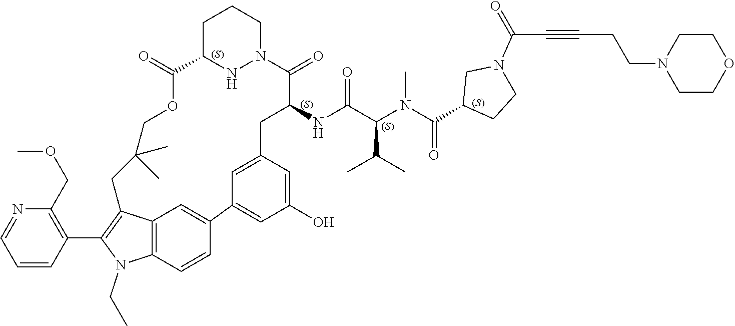 |
| BA 130 | 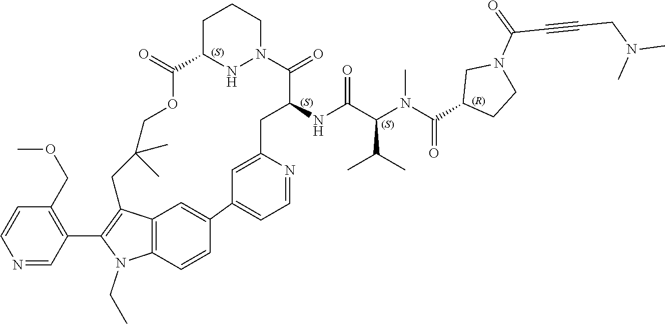 |
| BA 131 | 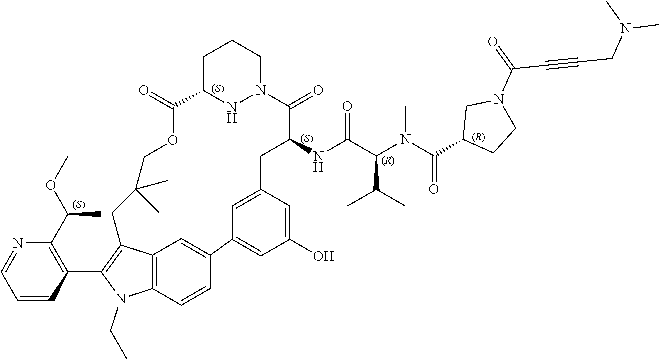 |
| BA 132 | 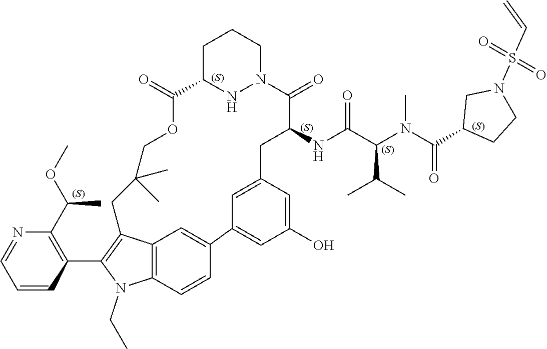 |
| BA 133 | 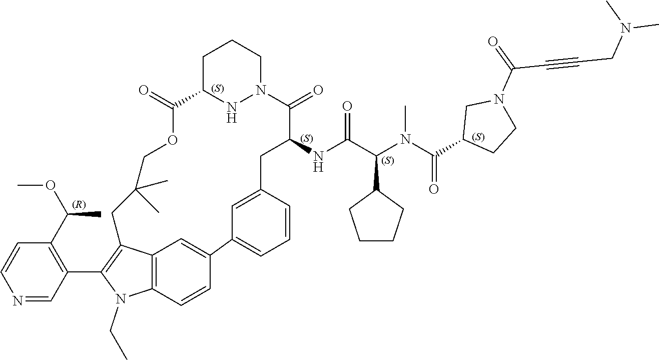 |
| BA 134 | 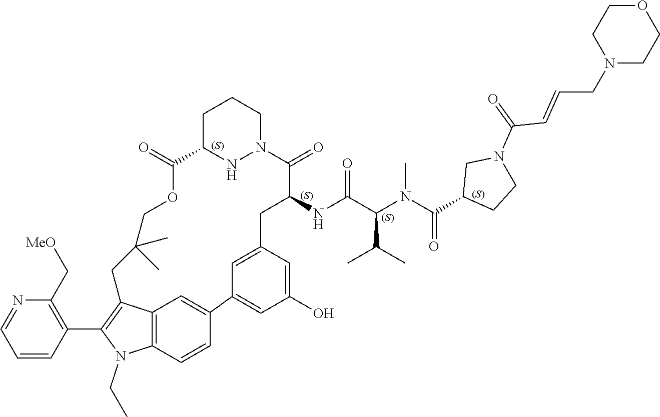 |
| BA 135 | 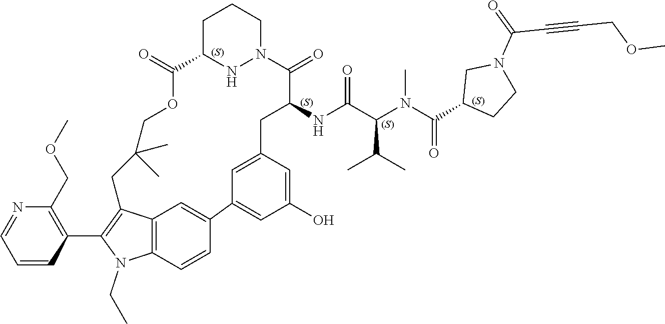 |
| BA 136 | 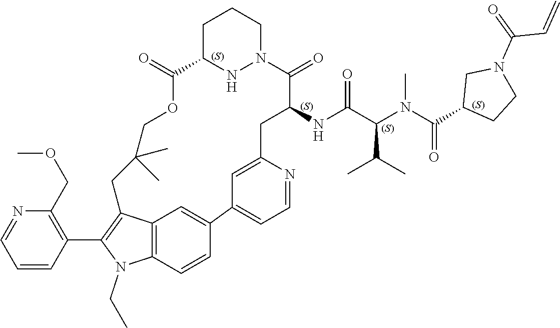 |
| BA 137 | 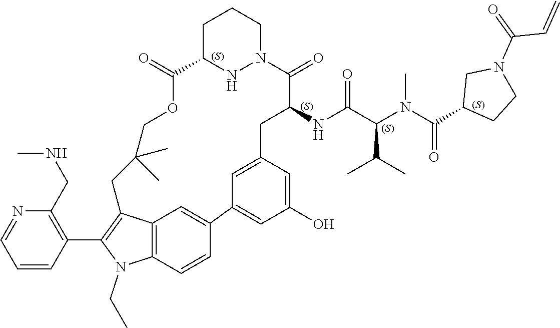 |
| BA 138 | 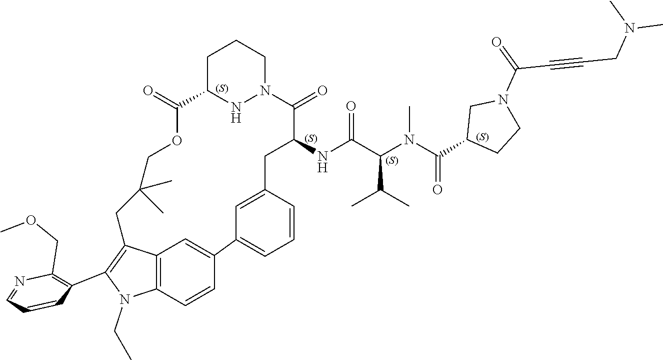 |
| BA 139 | 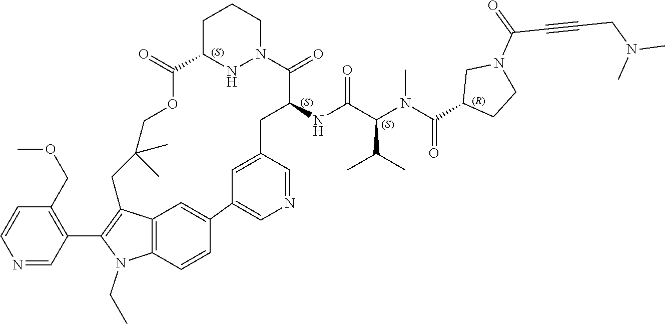 |
| BA 140 | 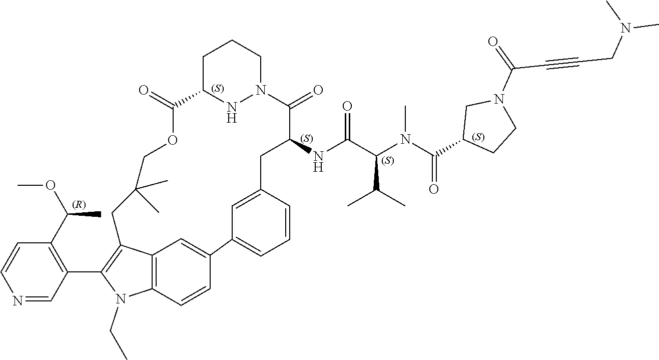 |
| BA 141 | 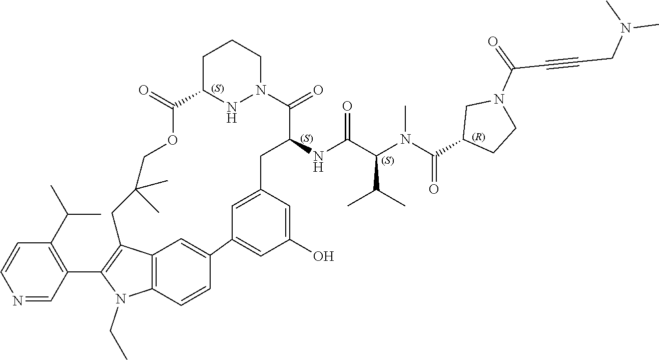 |
| BA 142 | 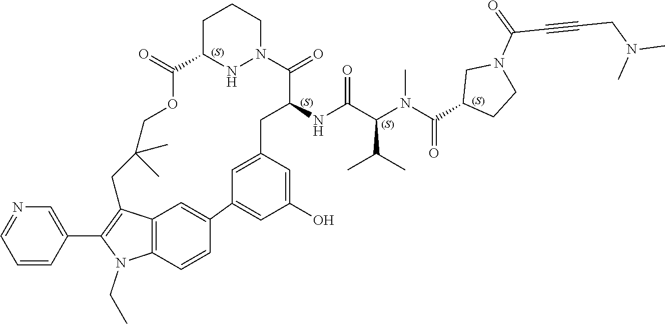 |
| BA 143 | 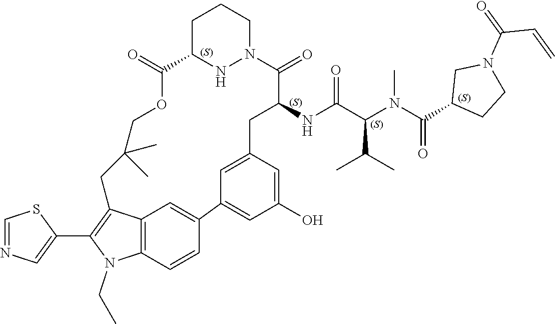 |
| BA 144 | 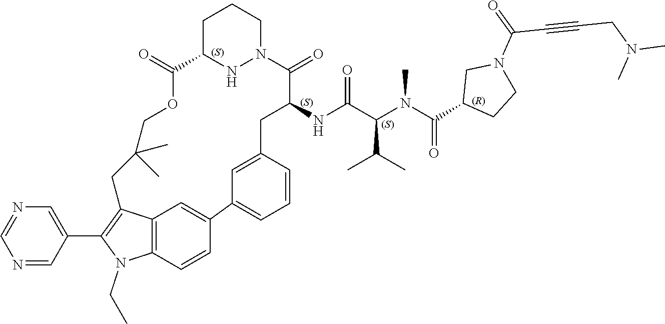 |
| BA 145 | 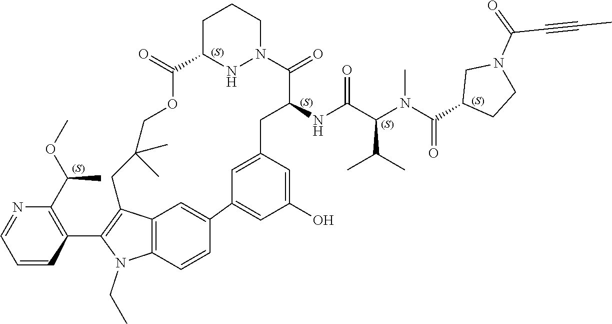 |
| BA 146 | 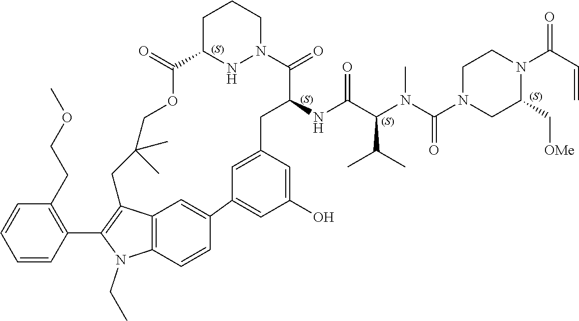 |
| BA 147 | 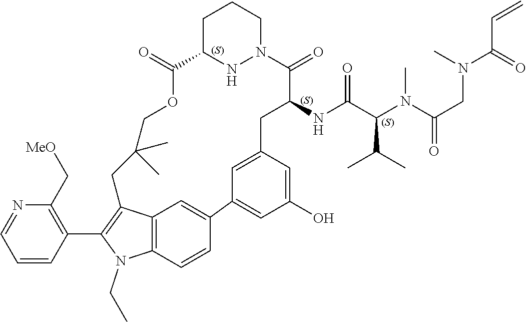 |
| BA 148 | 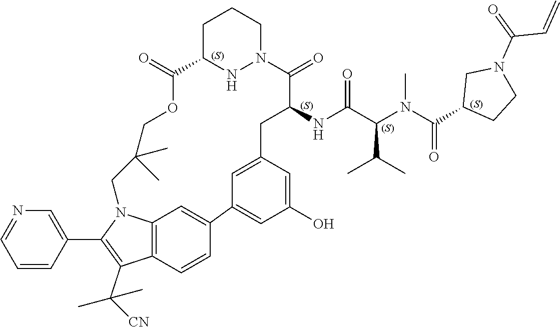 |
| BA 149 | 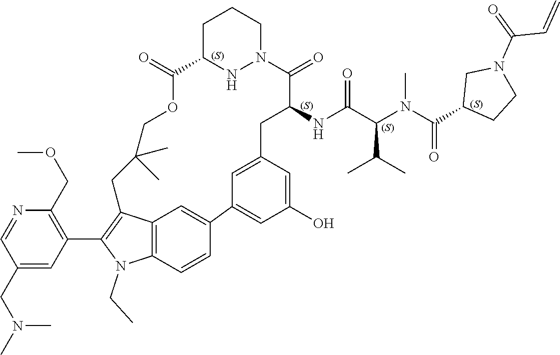 |
| BA 150 | 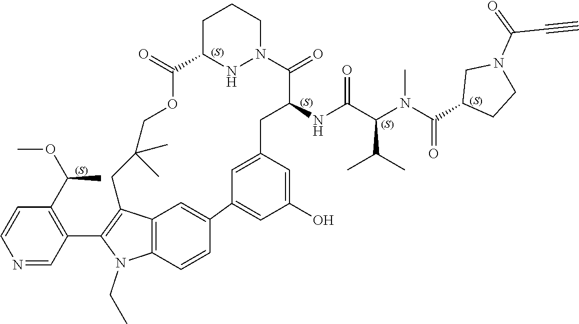 |
| BA 151 | 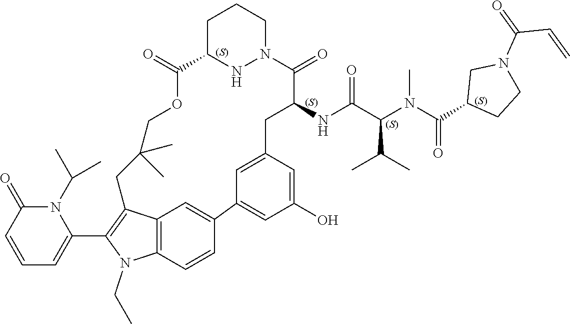 |
| BA 152 | 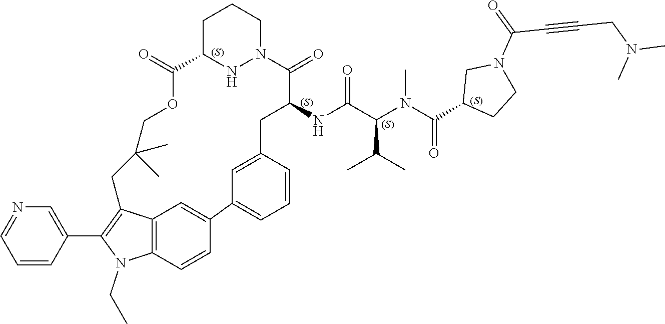 |
| BA 153 | 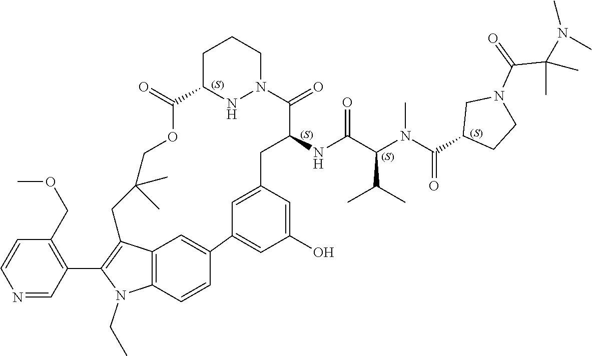 |
| BA 154 | 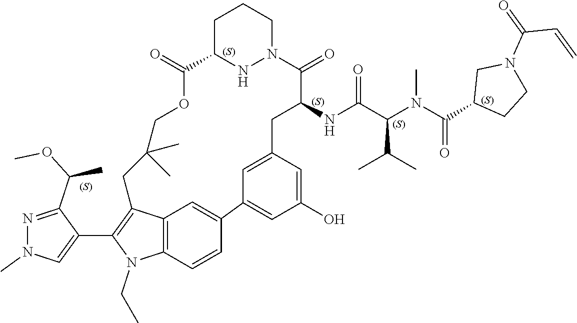 |
| BA 155 | 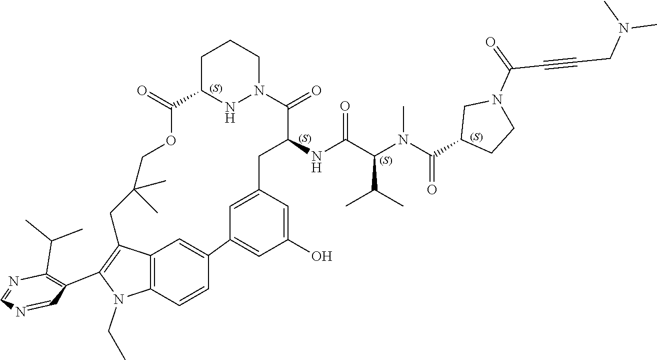 |
| BA 156 | 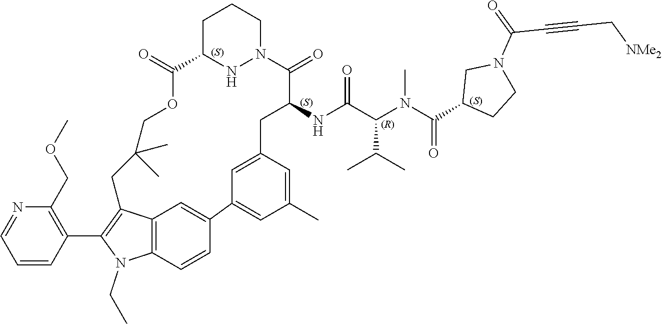 |
| BA 157 | 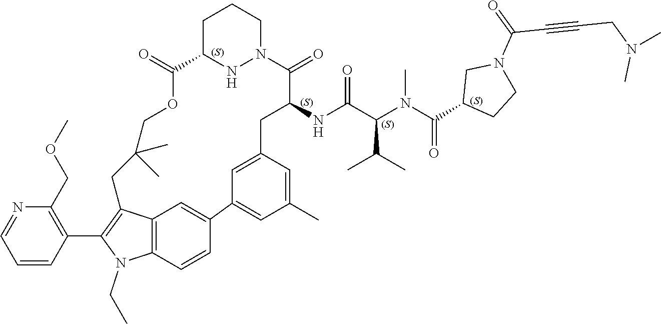 |
| BA 158 | 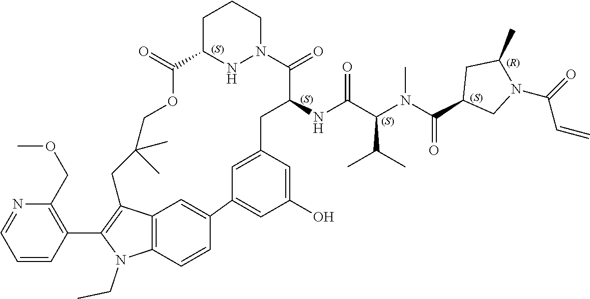 |
| BA 159 | 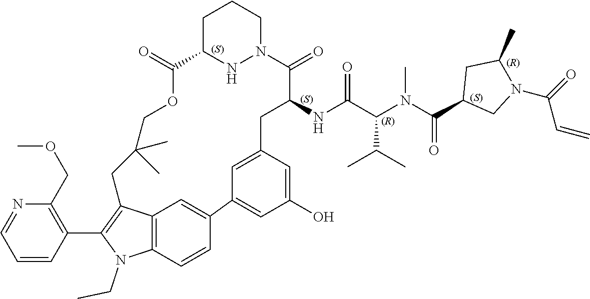 |
| BA 160 | 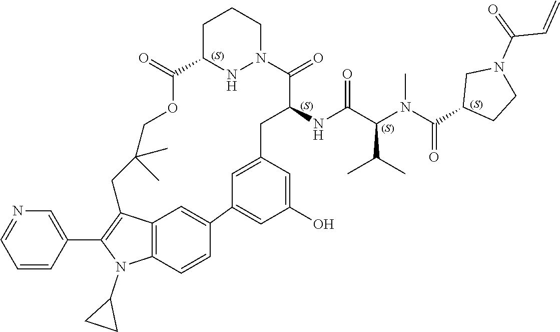 |
| BA 161 | 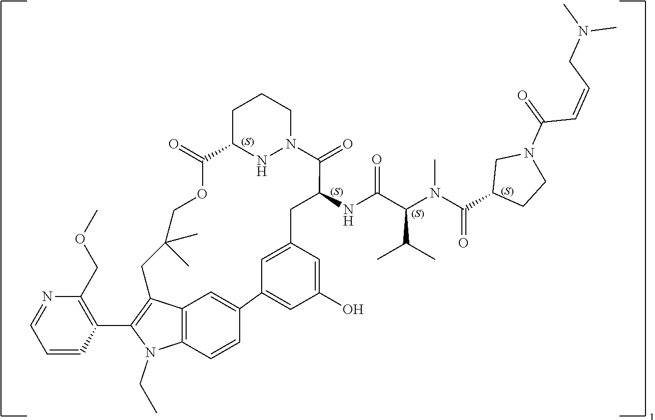 |
| BA 162 | 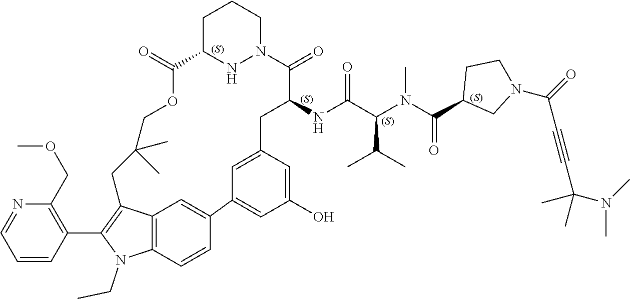 |
| BA 163 | 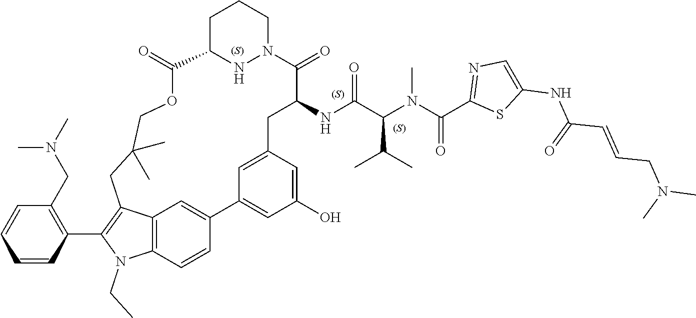 |
| BA 164 | 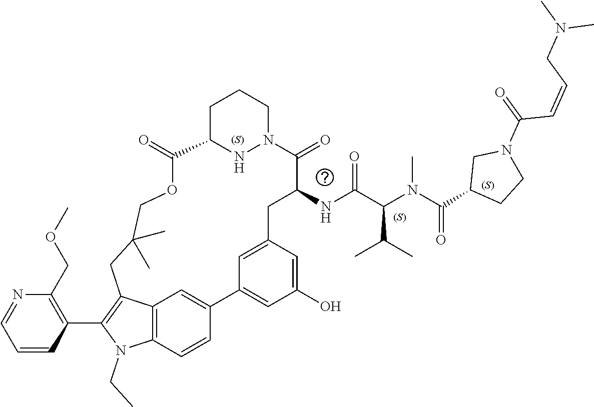 |
| BA 165 | 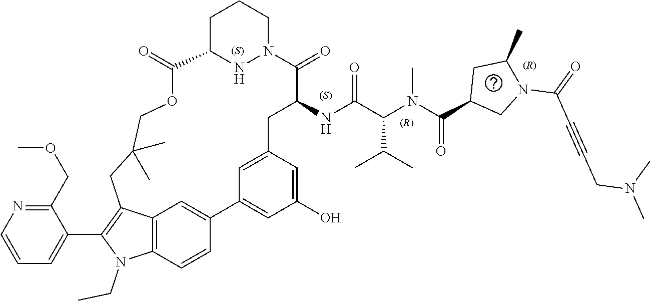 |
| BA 166 | 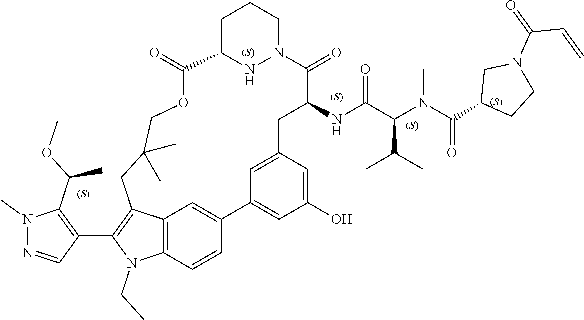 |
| BA 167 | 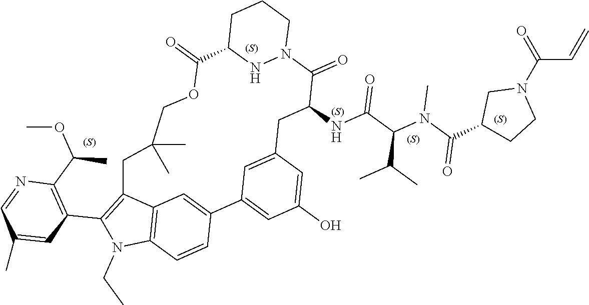 |
| BA 168 | 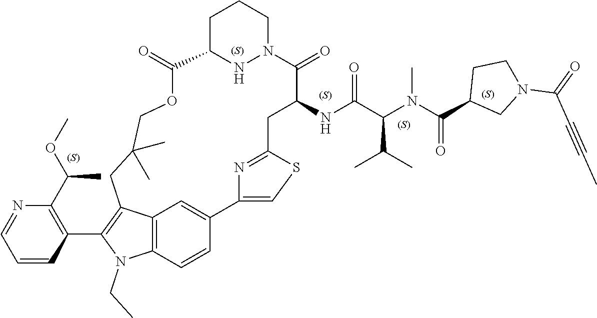 |
| BA 169 | 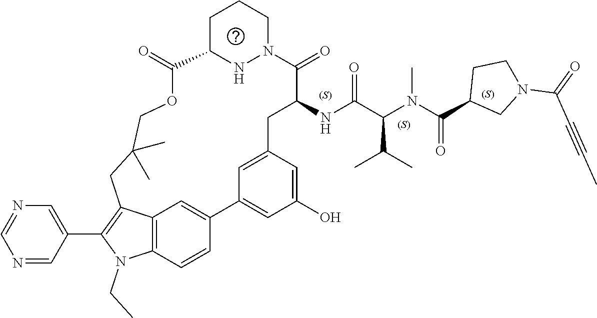 |
| BA 170 | 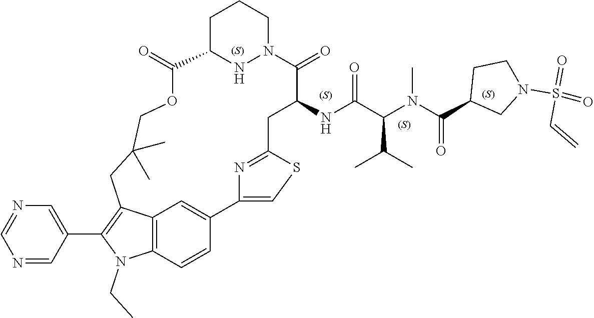 |
| BA 171 | 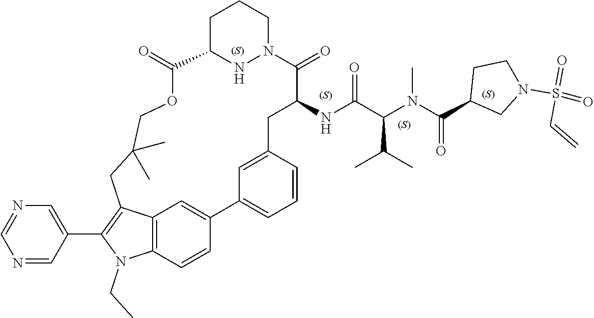 |
| BA 172 | 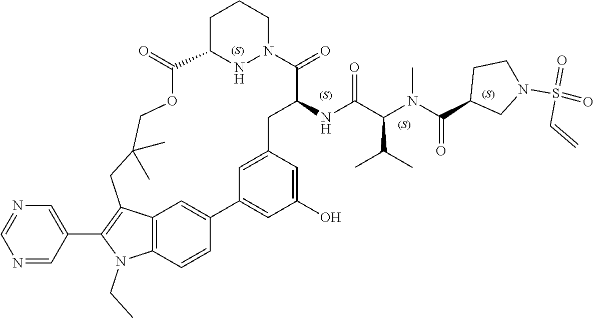 |
| BA 173 | 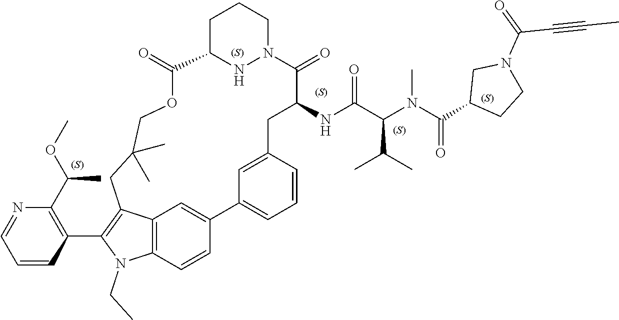 |
| BA 174 | 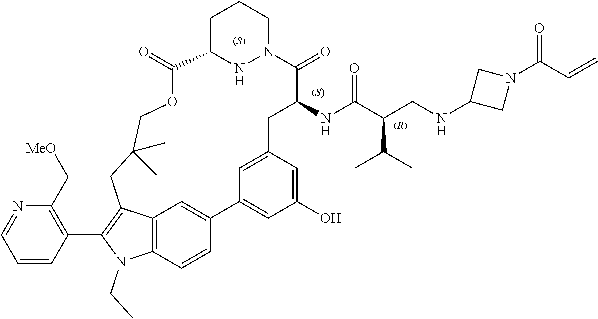 |
| BA 175 | 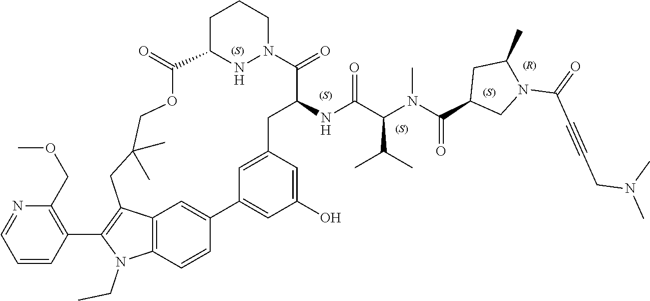 |
| BA 176 | 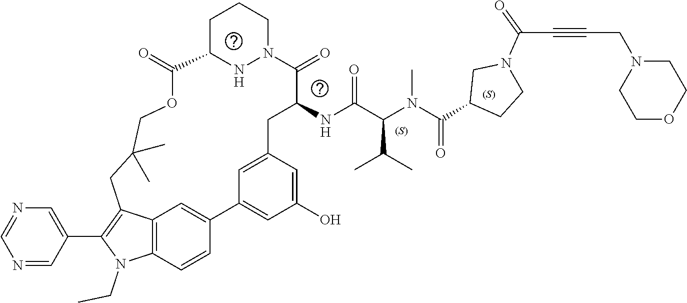 |
| BA 177 | 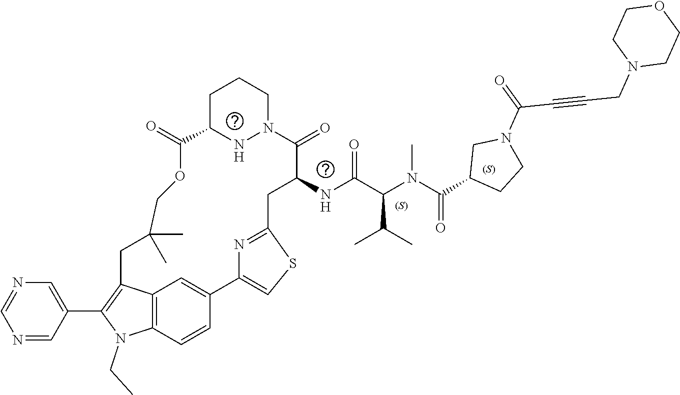 |
| BA 178 | 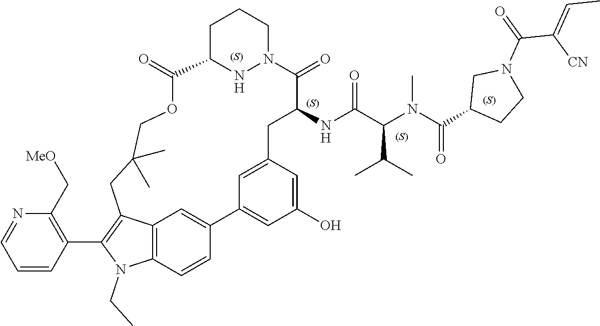 |
| BA 179 | 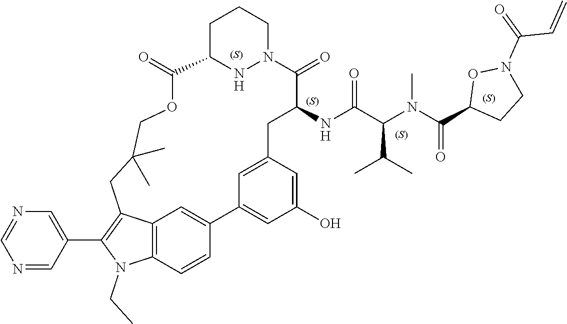 |
| BA 180 | 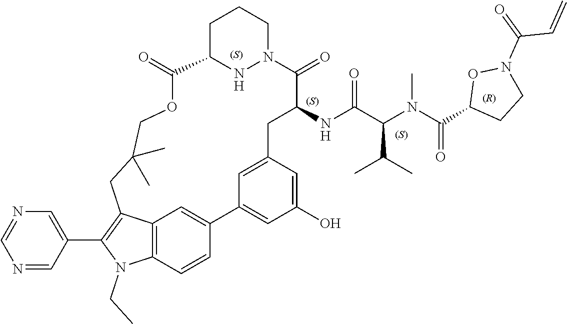 |
| BA 181 | 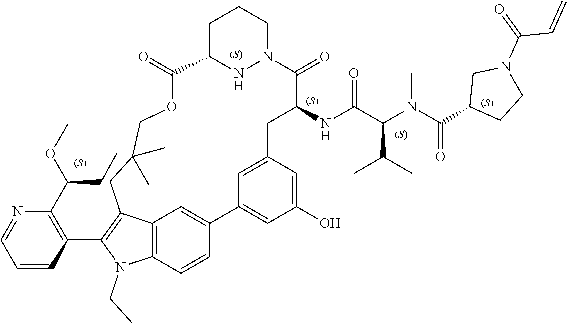 |
| BA 182 | 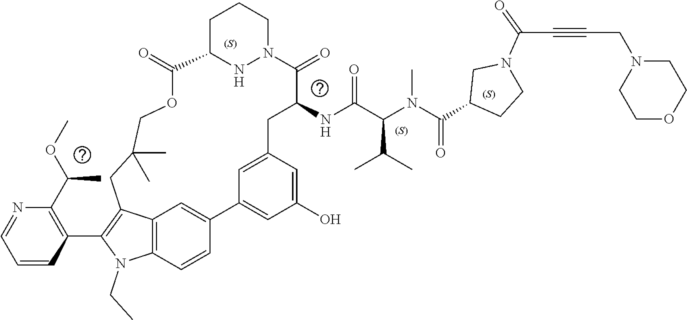 |
| BA 183 | 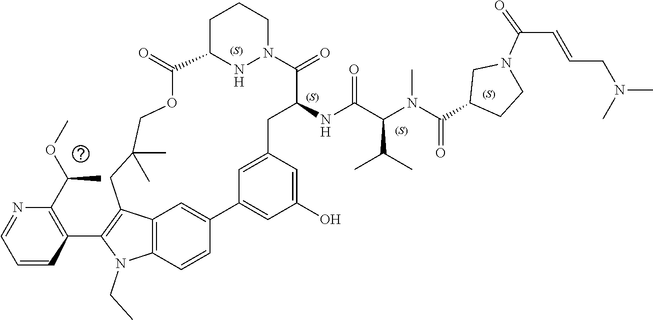 |
| BA 184 | 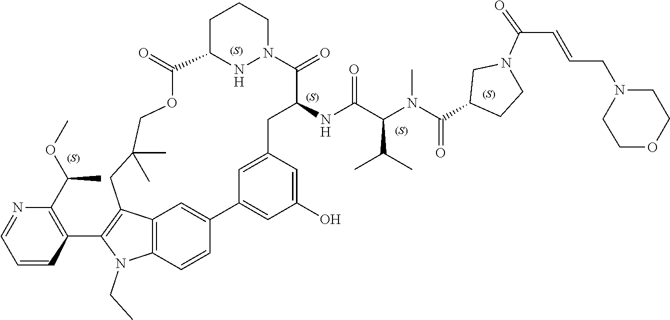 |
| BA 185 | 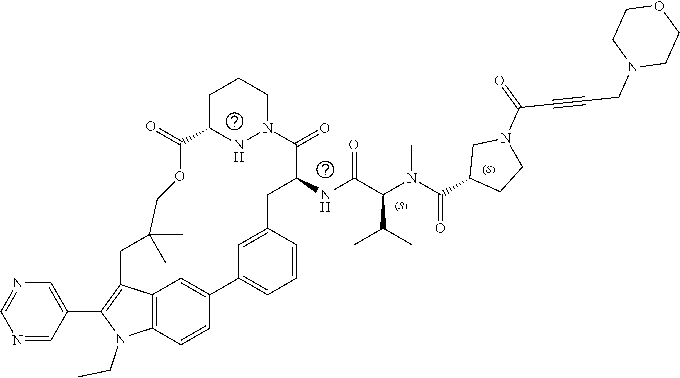 |
| BA 186 | 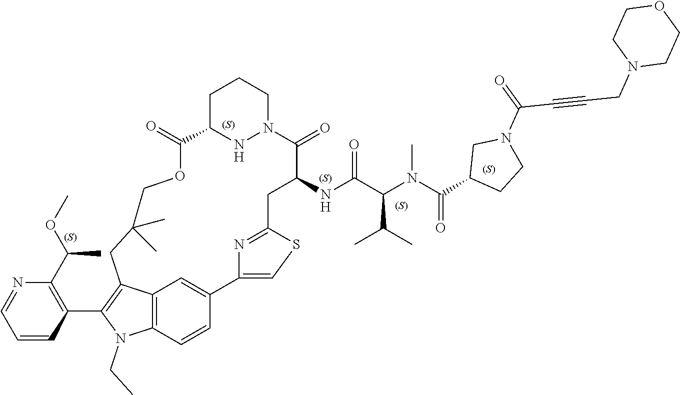 |
| BA 187 | 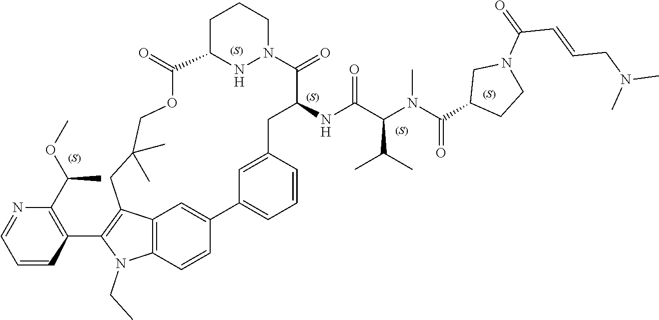 |
| BA 188 | 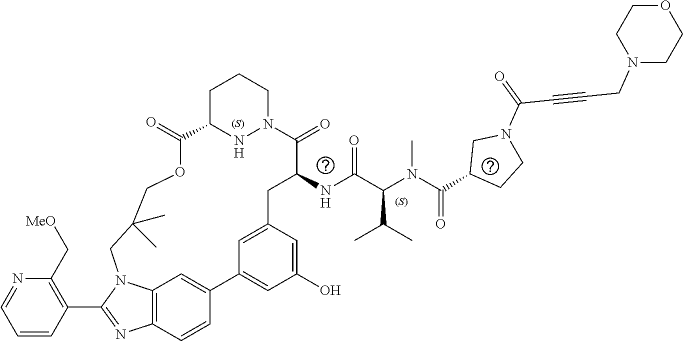 |
| BA 189 | 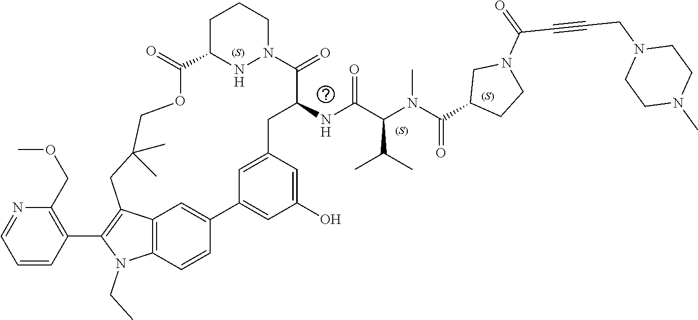 |
| BA 190 | 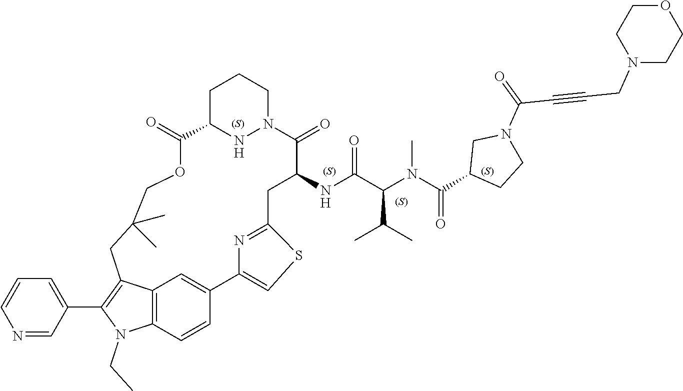 |
| BA 191 | 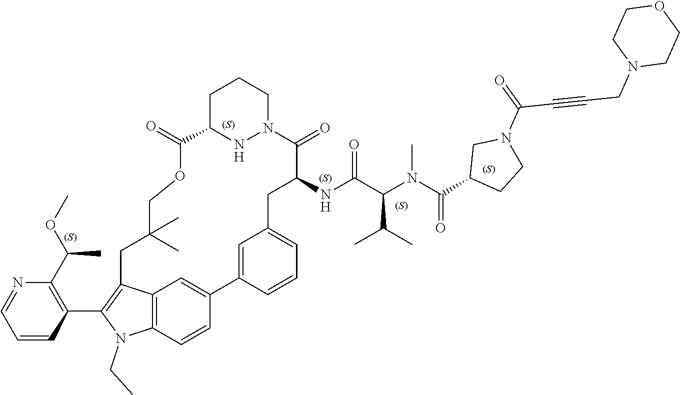 |
| BA 192 | 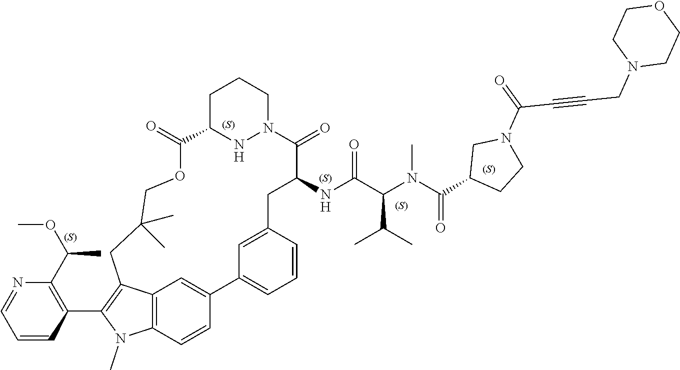 |
| BA 193 | 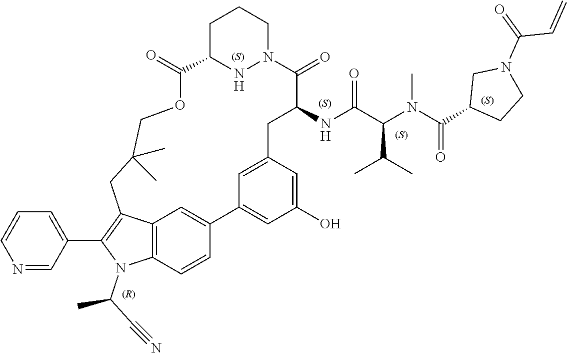 |
| BA 194 | 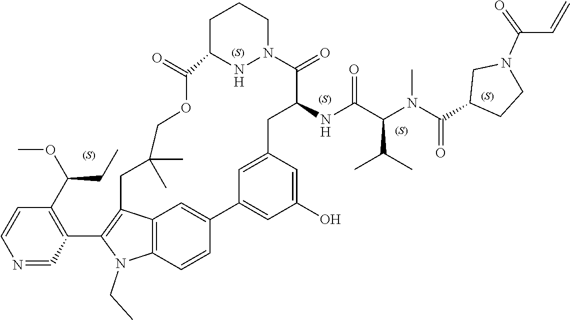 |
| BA 195 | 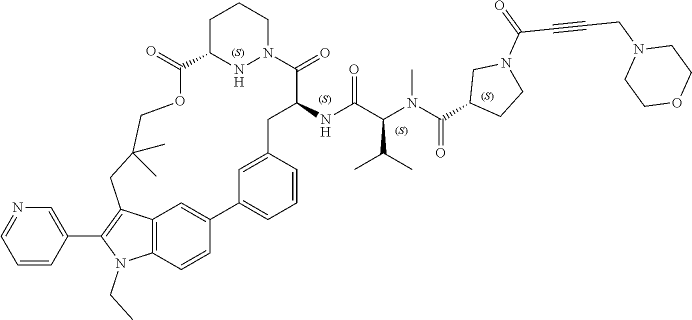 |
| BA 196 | 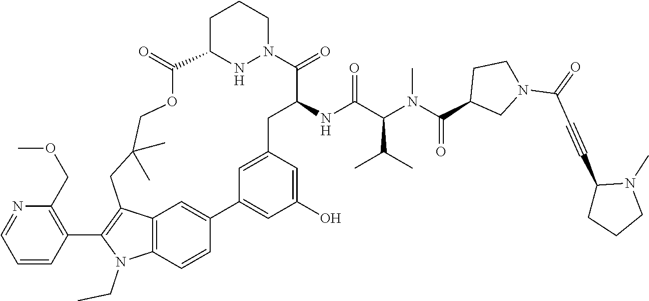 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| BA 197 | 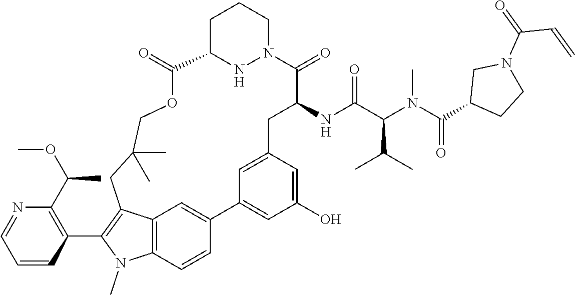 |
| BA 198 | 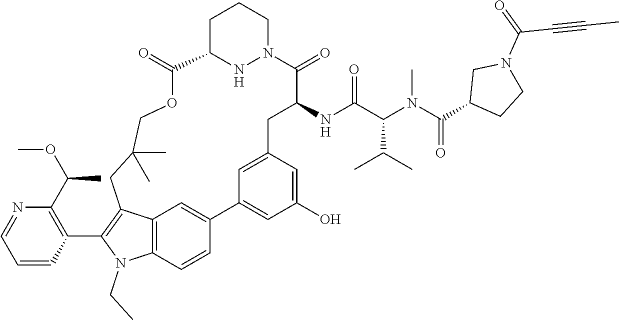 |
| BA 199 | 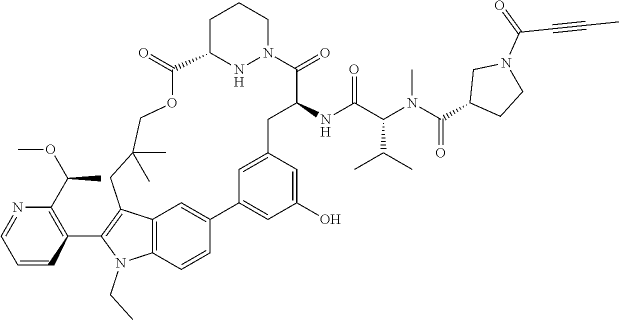 |
| BA 200 | 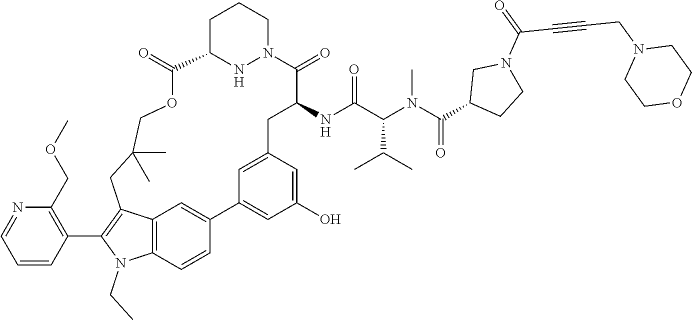 [SMILES: CCn1c(-c2cccnc2COC)c2c3cc(ccc31)-c1cc(O)cc(c1)C[C@H](NC(=O)[C@@H](C(C)C)N(C)C(=O)[C@H]1CCN(C(=O)C#CCN3CCOCC3)C1)C(=O)N1CCC[C@H](N1)C(=O)OCC(C)(C)C2] |
| BA 201 | 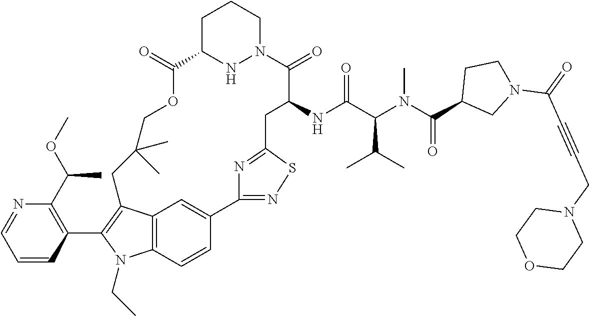 |
| BA 202 | 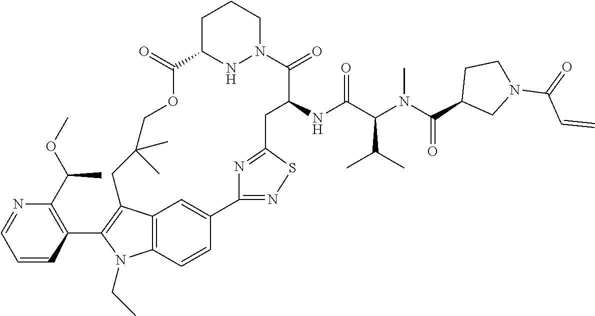 |
| BA 203 | 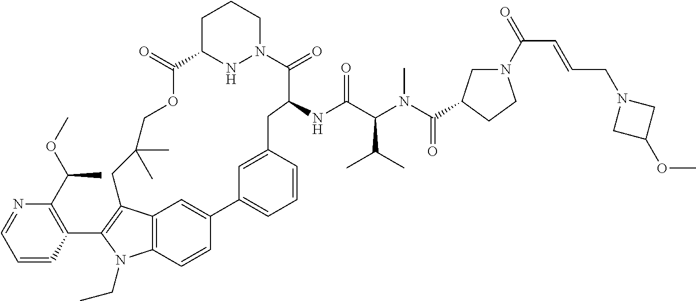 |
| BA 204 | 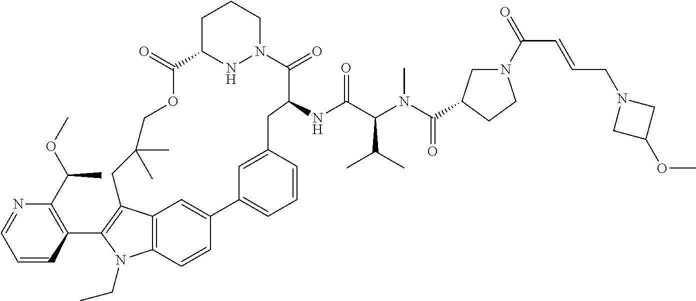 |
| BA 205 | 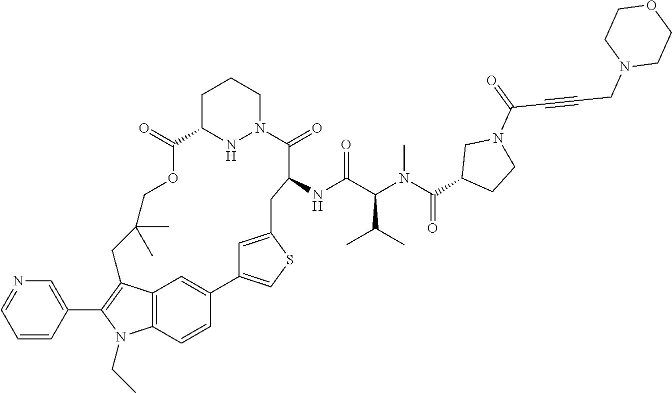 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BA 206 | 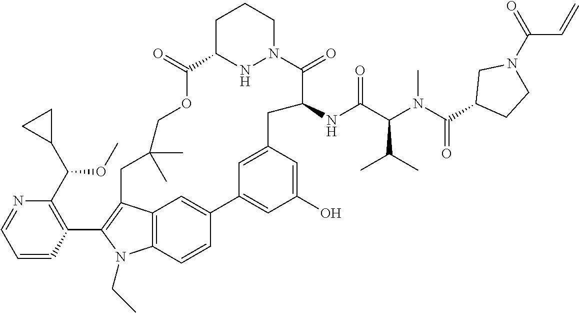 |
| BA 207 | 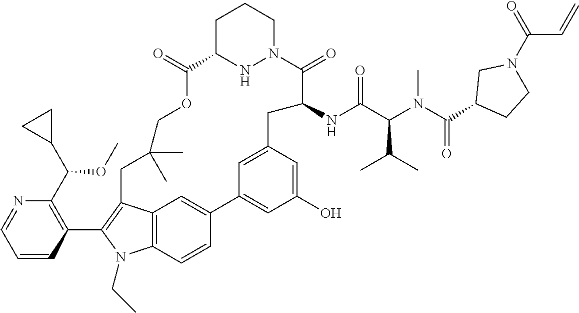 |
| BA 208 | 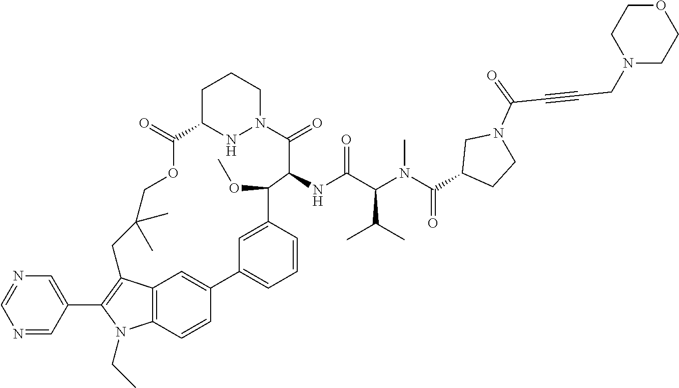 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BA 209 | 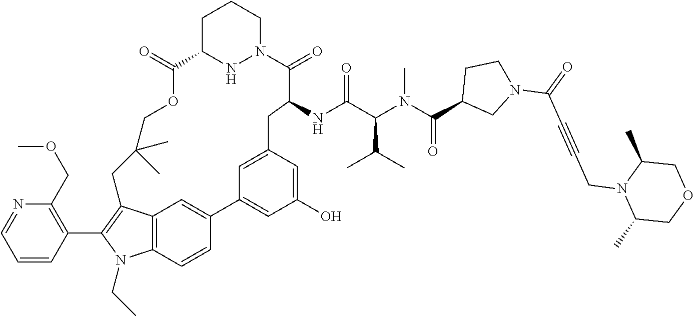 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3[C@@H](C)COC[C@@H]3C)C1)C(=O)N1CCC[C@H](N1)C(=O)OCC(C)(C)C2] |
| BA 210 | 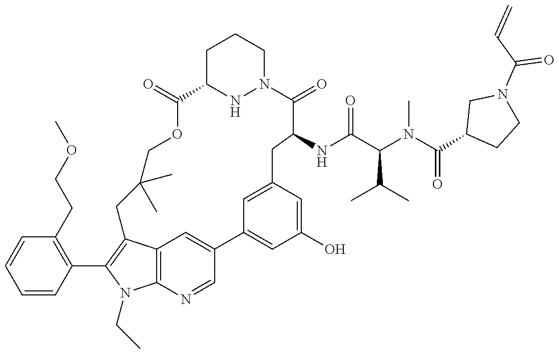 [SMILES: C=CC(=O)N1CC[C@H](C(=O)N(C)[C@H](C(=O)N[C@H]2Cc3cc(O)cc(c3)-c3cnc4c(c3)c(c(-c3ccccc3CCOC)n4CC)CC(C)(C)COC(=O)[C@@H]3CCCN(N3)C2=O)C(C)C)C1] |
| BA 211 | 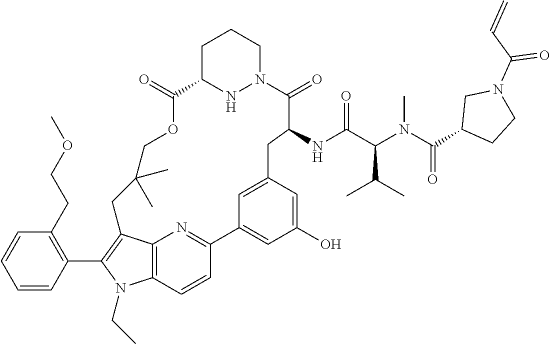 [SMILES: C=CC(=O)N1CC[C@H](C(=O)N(C)[C@H](C(=O)N[C@H]2Cc3cc(O)cc(c3)-c3ccc4c(n3)c(c(-c3ccccc3CCOC)n4CC)CC(C)(C)COC(=O)[C@@H]3CCCN(N3)C2=O)C(C)C)C1] |
| BA 212 | 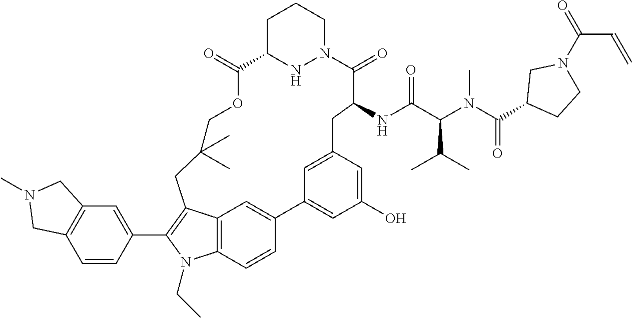 [SMILES: C=CC(=O)N1CC[C@H](C(=O)N(C)[C@H](C(=O)N[C@H]2Cc3cc(O)cc(c3)-c3ccc4c(c3)c(c(-c3ccc5c(c3)CN(C)C5)n4CC)CC(C)(C)COC(=O)[C@@H]3CCCN(N3)C2=O)C(C)C)C1] |
| BA 213 | 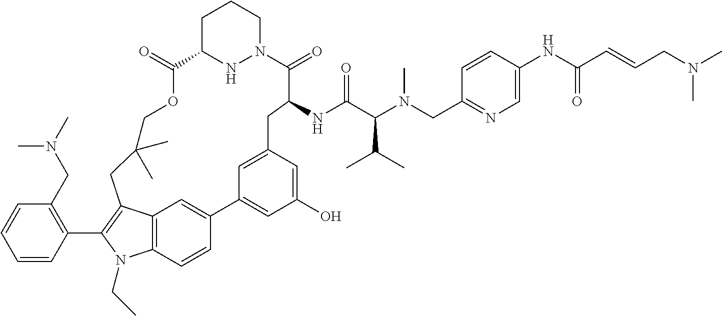 [SMILES: CCn1c(-c2ccccc2CN(C)C)c2c3cc(ccc31)-c1cc(O)cc(c1)C[C@H](NC(=O)[C@H](C(C)C)N(C)Cc1ccc(NC(=O)/C=C/CN(C)C)cn1)C(=O)N1CCC[C@H](N1)C(=O)OCC(C)(C)C2] |
| BA 214 | 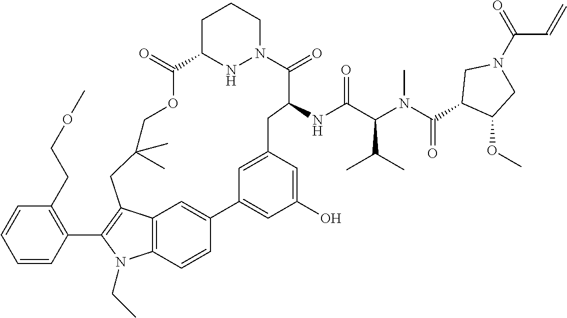 [SMILES: C=CC(=O)N1C[C@H](OC)[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| BA 215 | 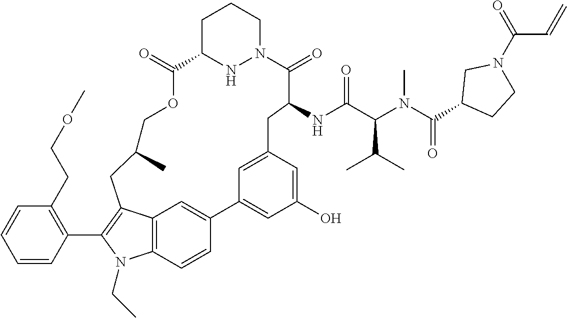 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)C[C@H](C)COC(=O)[C@@H]3CCCN(N3)C2=O)C(C)C)C1] |
| BA 216 | 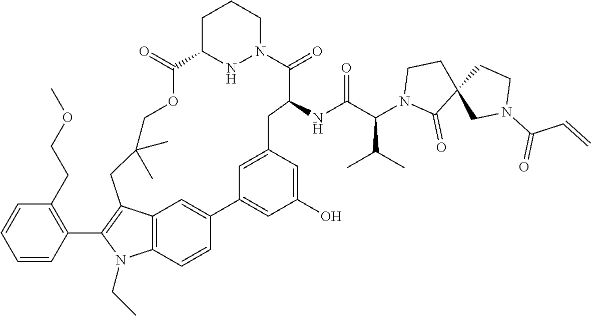 [SMILES: C=CC(=O)N1CC[C@]2(CCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| BA 217 | 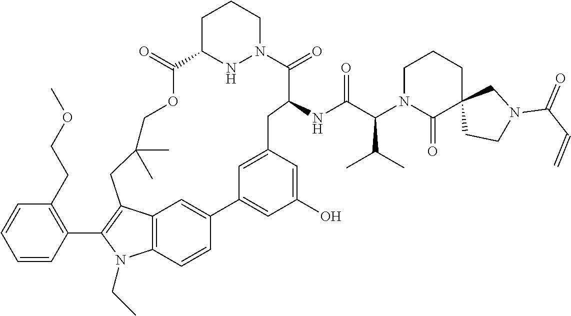 [SMILES: C=CC(=O)N1CC[C@]2(CCCN([C@H](C(=O)N[C@H]3Cc4cc(O)cc(c4)-c4ccc5c(c4)c(c(-c4ccccc4CCOC)n5CC)CC(C)(C)COC(=O)[C@@H]4CCCN(N4)C3=O)C(C)C)C2=O)C1] |
| BA 218 | 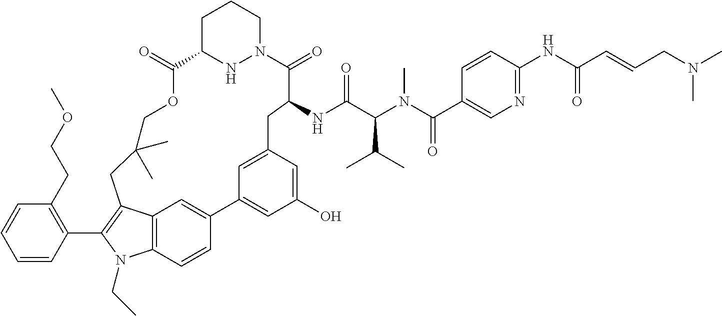 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)nc1)C(=O)N1CCC[C@H](N1)C(=O)OCC(C)(C)C2] |
| BA 219 | 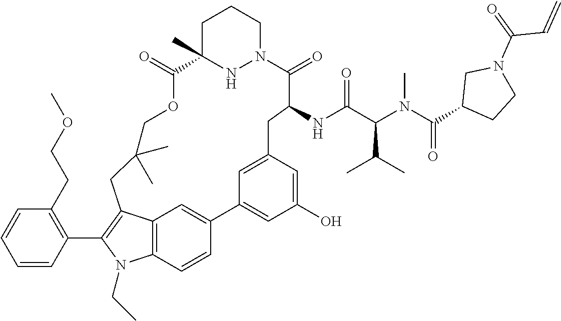 [SMILES: C=CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@]3(C)CCCN(N3)C2=O)C(C)C)C1] |
| BA 220 | 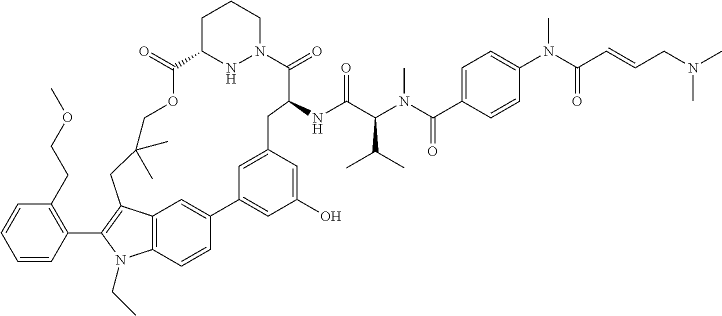 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(N(C)C(=O)/C=C/CN(C)C)cc1)C(=O)N1CCC[C@H](N1)C(=O)OCC(C)(C)C2] |
| BA 221 | 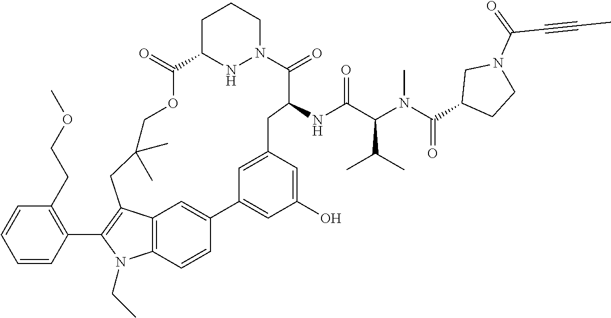 [SMILES: CC#CC(=O)N1CC[C@H](C(=O)N(C)[C@H](C(=O)N[C@H]2Cc3cc(O)cc(c3)-c3ccc4c(c3)c(c(-c3ccccc3CCOC)n4CC)CC(C)(C)COC(=O)[C@@H]3CCCN(N3)C2=O)C(C)C)C1] |
| BA 222 | 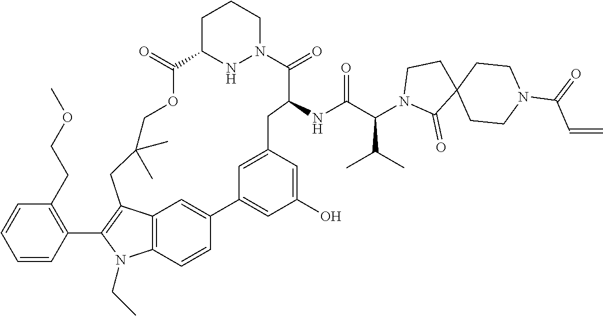 [SMILES: C=CC(=O)N1CCC2(CC1)CCN([C@H](C(=O)N[C@H]1Cc3cc(O)cc(c3)-c3ccc4c(c3)c(c(-c3ccccc3CCOC)n4CC)CC(C)(C)COC(=O)[C@@H]3CCCN(N3)C1=O)C(C)C)C2=O] |
| BA 223 | 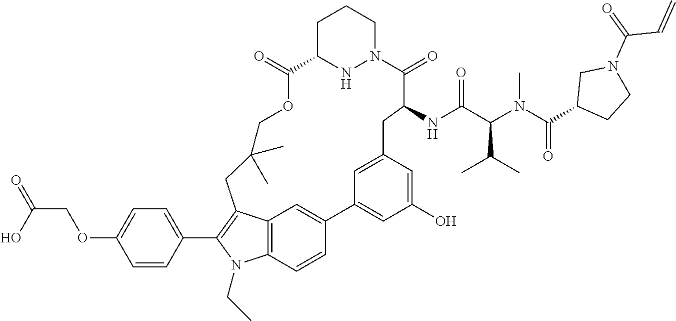 [SMILES: C=CC(=O)N1CC[C@H](C(=O)N(C)[C@H](C(=O)N[C@H]2Cc3cc(O)cc(c3)-c3ccc4c(c3)c(c(-c3ccc(OCC(=O)O)cc3)n4CC)CC(C)(C)COC(=O)[C@@H]3CCCN(N3)C2=O)C(C)C)C1] |
| BA 224 | 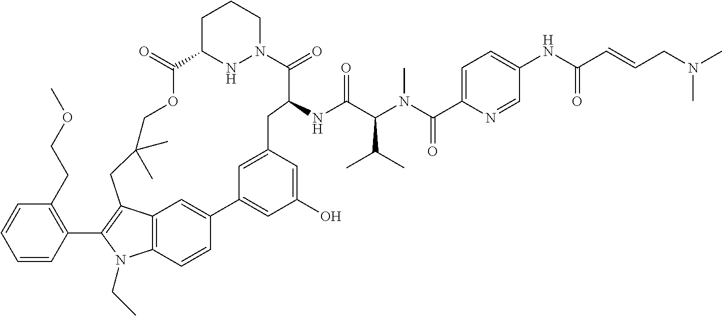 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cn1)C(=O)N1CCC[C@H](N1)C(=O)OCC(C)(C)C2] |
| BA 225 | 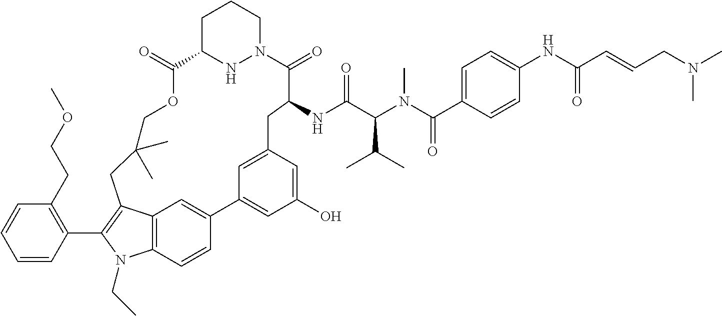 [SMILES: CCn1c(-c2ccccc2CCOC)c2c3cc(ccc31)-c1cc(O)cc(c1)C[C@H](NC(=O)[C@H](C(C)C)N(C)C(=O)c1ccc(NC(=O)/C=C/CN(C)C)cc1)C(=O)N1CCC[C@H](N1)C(=O)OCC(C)(C)C2] |
| BA 226 | 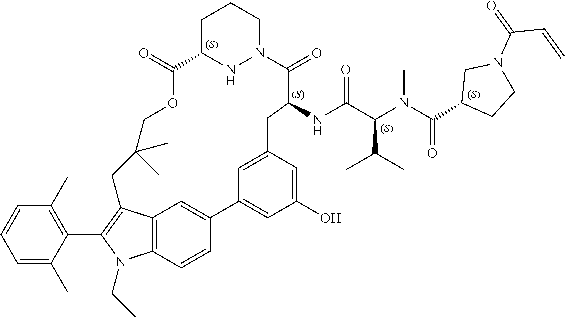 |
| BA 227 | 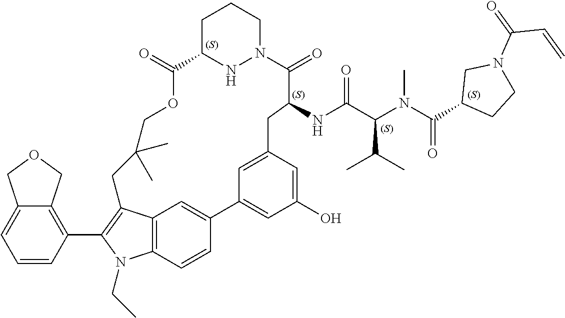 |
| BA 228 | 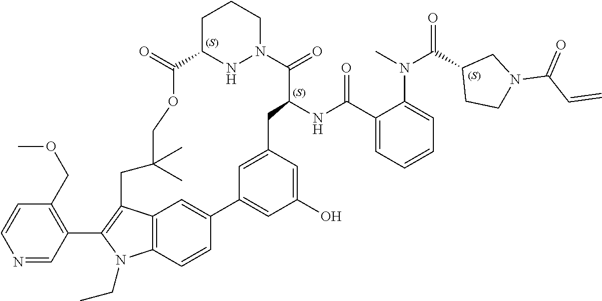 |
| BA 229 | 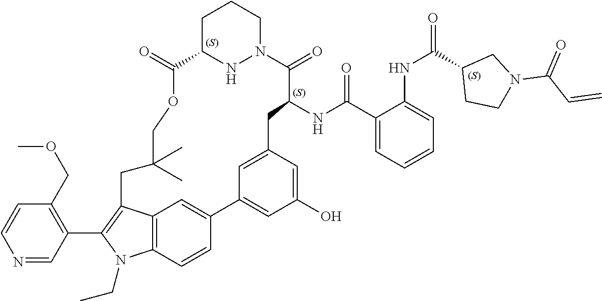 |
| BA 230 | 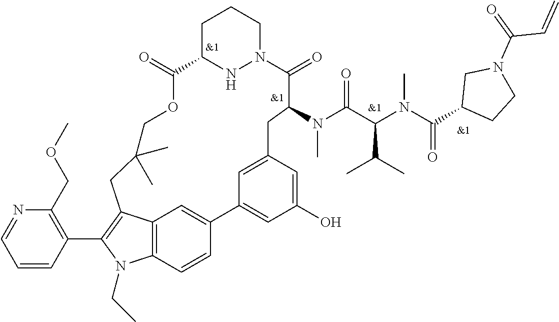 [SMILES: C=CC(=O)N1CC[C@H](C(=O)N(C)[C@H](C(=O)N(C)[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| BA 231 | 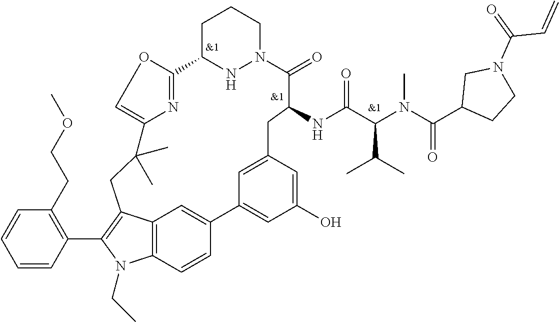 [SMILES: C=CC(=O)N1CCC(C(=O)N(C)[C@H](C(=O)N[C@H]2Cc3cc(O)cc(c3)-c3ccc4c(c3)c(c(-c3ccccc3CCOC)n4CC)CC(C)(C)c3coc(n3)[C@@H]3CCCN(N3)C2=O)C(C)C)C1] |
| BA 232 | 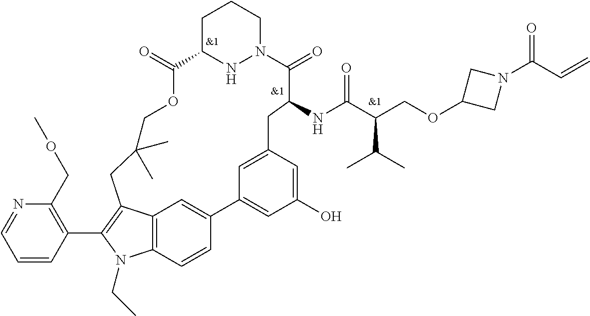 [SMILES: C=CC(=O)N1CC(OC[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| BA 233 | 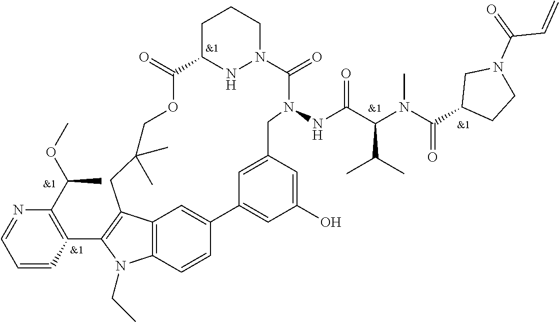 |
| BA 234 | 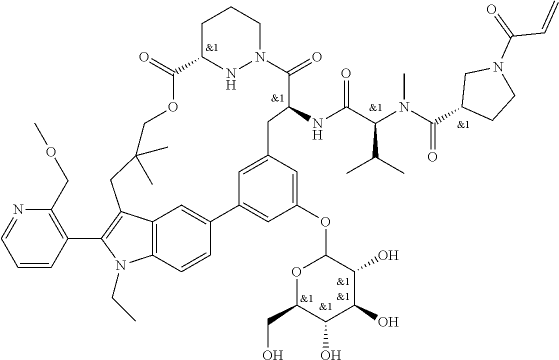 [SMILES: C=CC(=O)N1CC[C@H](C(=O)N(C)[C@H](C(=O)N[C@H]2Cc3cc(OC4O[C@H](CO)[C@@H](O)[C@H](O)[C@H]4O)cc(c3)-c3ccc4c(c3)c(c(-c3cccnc3COC)n4CC)CC(C)(C)COC(=O)[C@@H]3CCCN(N3)C2=O)C(C)C)C1] |
| BA 235 | 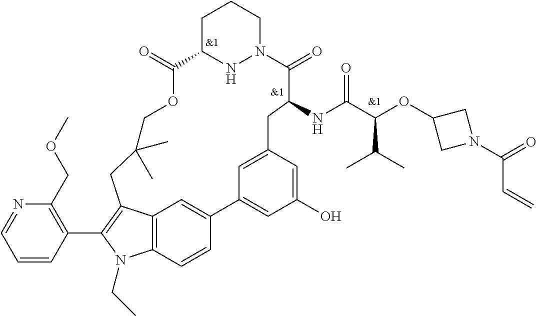 [SMILES: C=CC(=O)N1CC(O[C@H](C(=O)N[C@H]2Cc3cc(O)cc(c3)-c3ccc4c(c3)c(c(-c3cccnc3COC)n4CC)CC(C)(C)COC(=O)[C@@H]3CCCN(N3)C2=O)C(C)C)C1] |
| BA 236 | 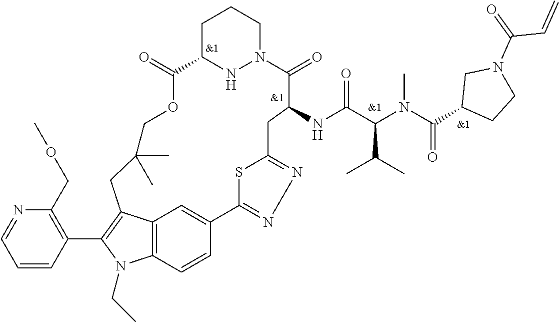 [SMILES: C=CC(=O)N1CC[C@H](C(=O)N(C)[C@H](C(=O)N[C@H]2CC3=NN=C(S3)c3ccc4c(c3)c(c(-c3cccnc3COC)n4CC)CC(C)(C)COC(=O)[C@@H]3CCCN(N3)C2=O)C(C)C)C1] |
| BA 237 | 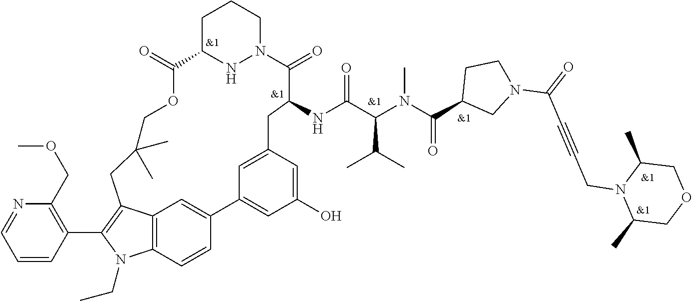 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CCN3[C@H](C)COC[C@@H]3C)C1)C(=O)N1CCC[C@H](N1)C(=O)OCC(C)(C)C2] |
| BA 238 | 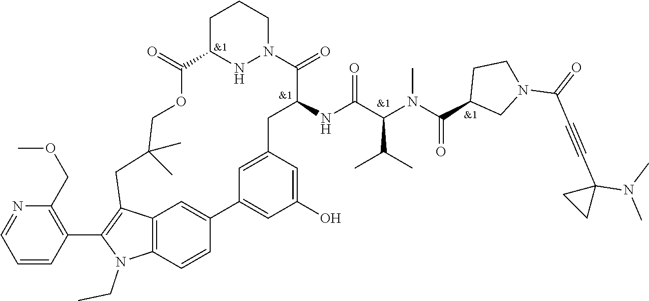 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#CC3(N(C)C)CC3)C1)C(=O)N1CCC[C@H](N1)C(=O)OCC(C)(C)C2] |
| BA 239 | 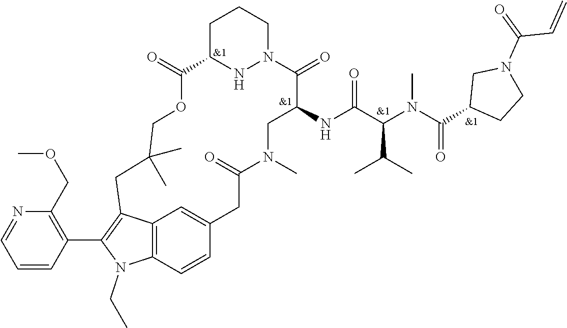 |
| BA 240 | 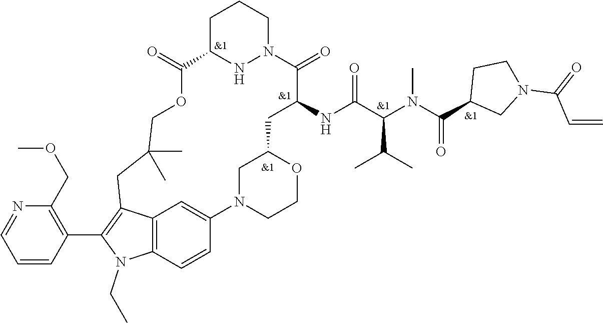 |
| BA 241 | 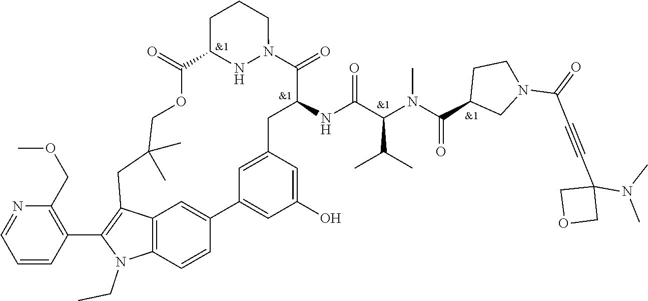 |
| BA 242 | 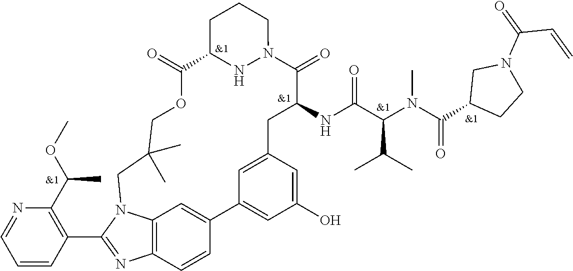 |
| BA 243 | 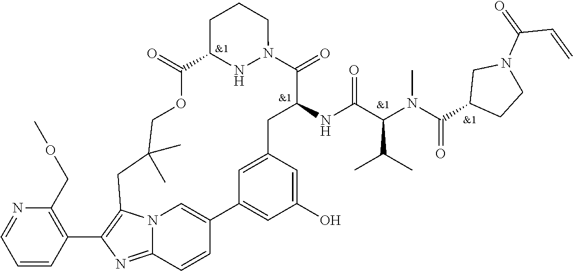 |
| BA 244 | 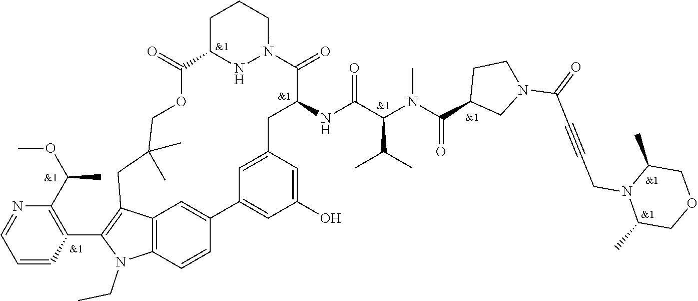 |
| BA 245 | 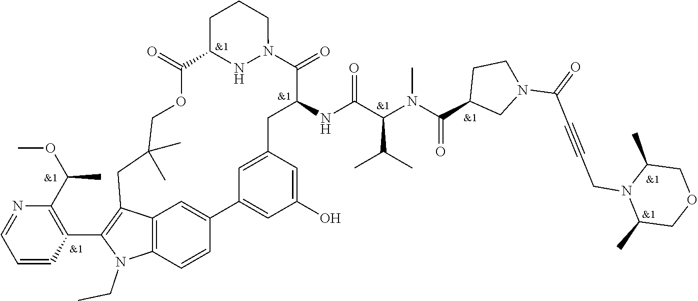 |
| BA 246 | 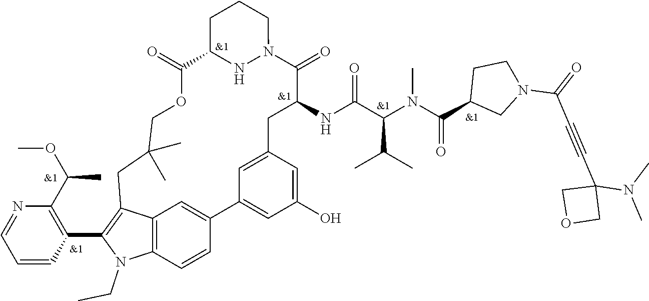 |
| BA 247 | 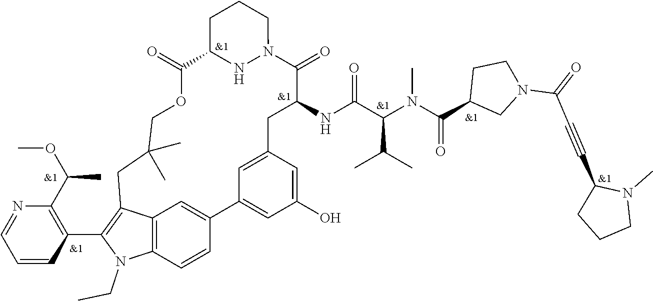 |
| BA 248 | 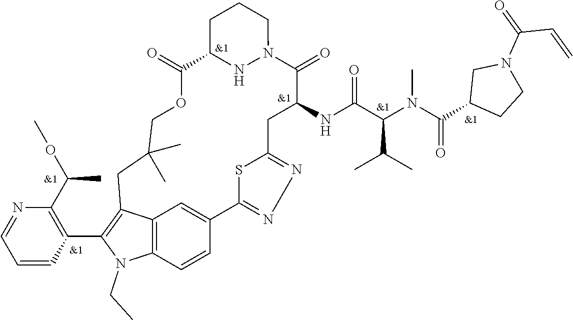 |
| BA 249 | 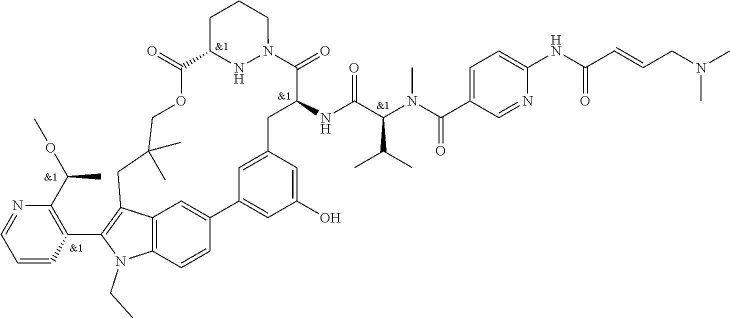 |
| BA 250 | 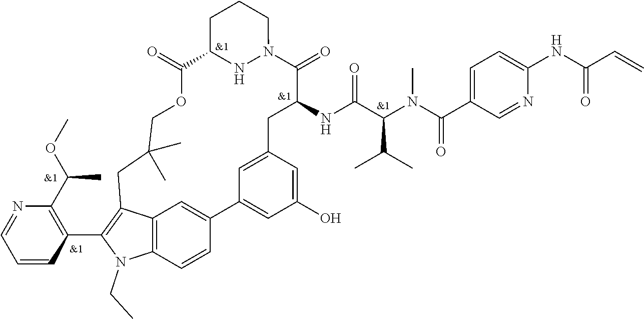 |
| BA 251 | 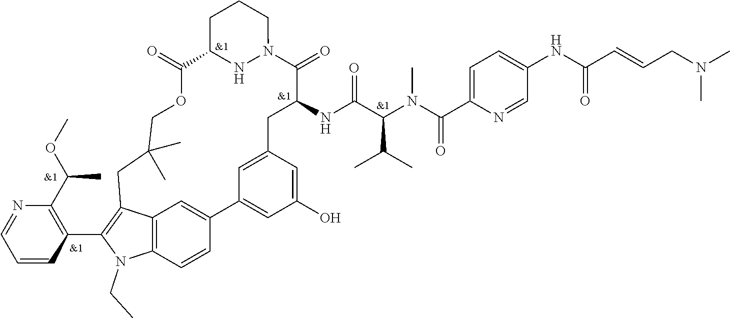 |
| BA 252 | 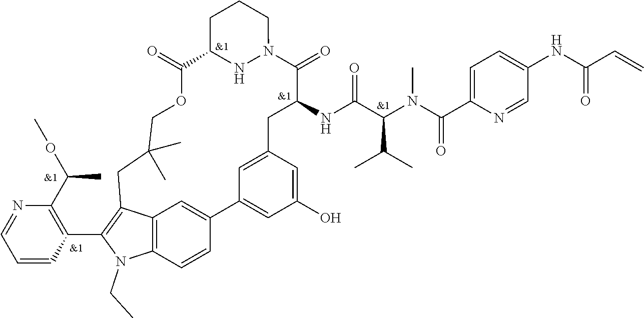 |
| BA 253 | 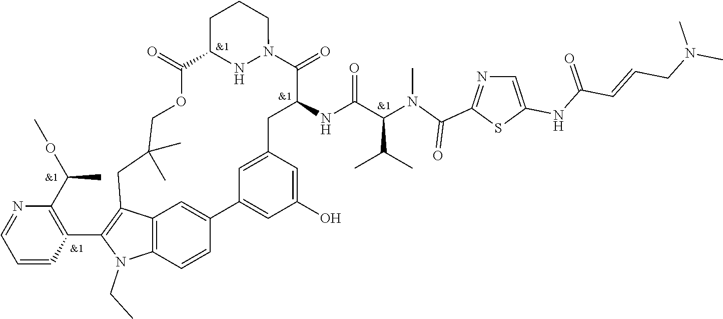 |
| BA 254 | 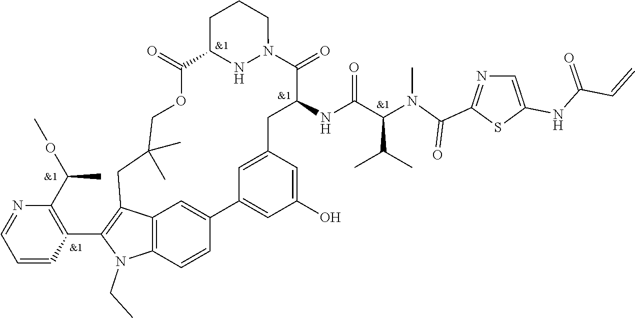 |
| BA 255 | 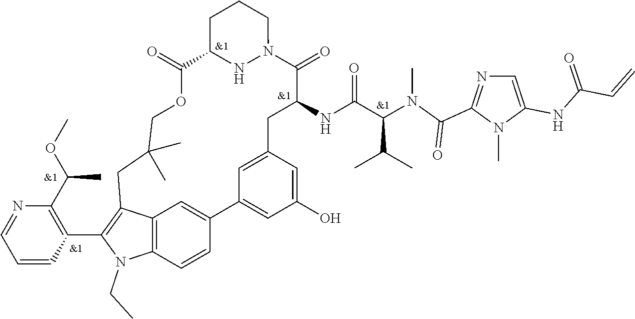 |
| BA 256 | 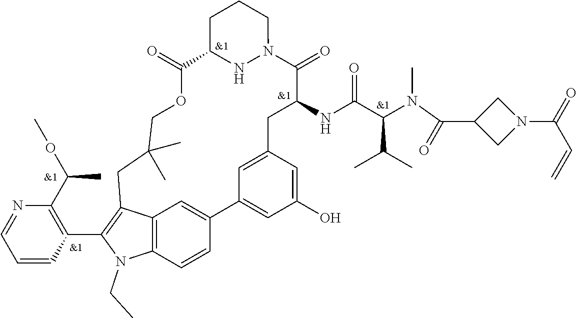 |
| BA 257 | 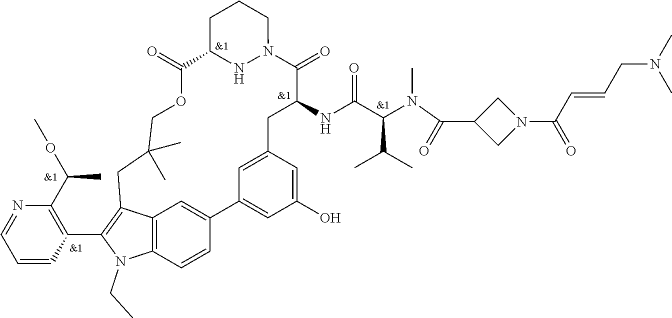 |
| BA 258 | 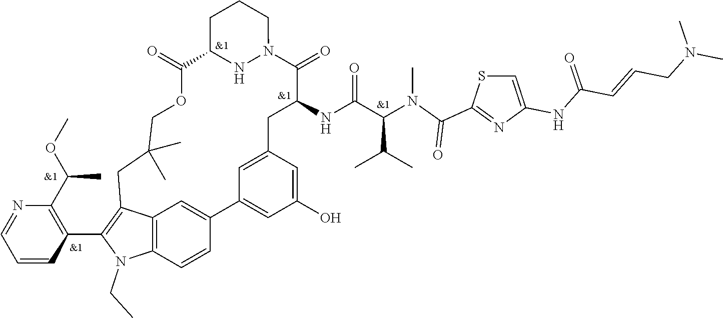 |
| BA 259 | 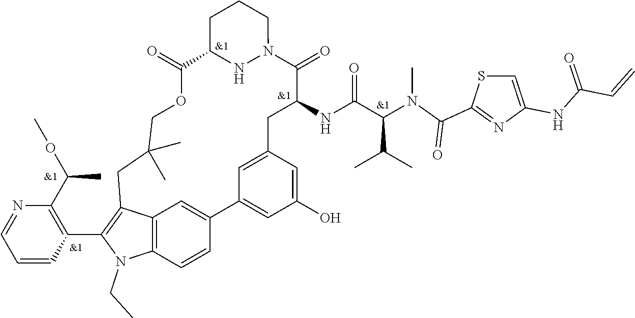 |
| BA 260 | 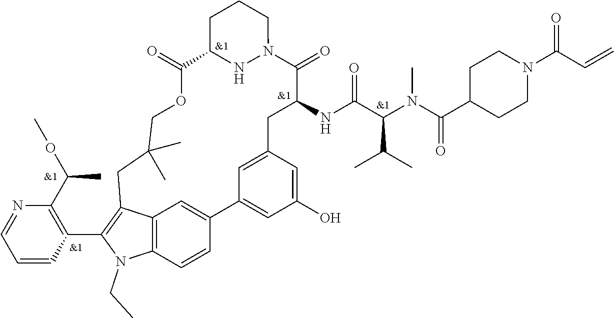 |
| BA 261 | 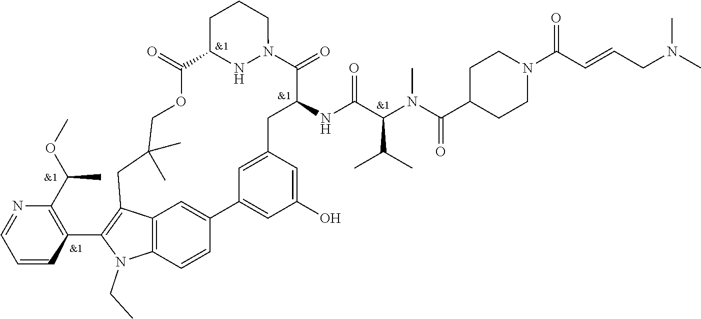 |
| BA 262 | 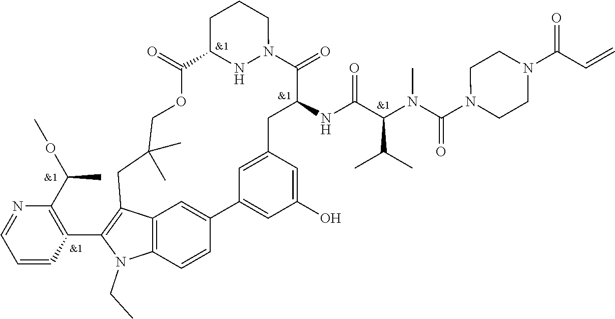 |
| BA 263 | 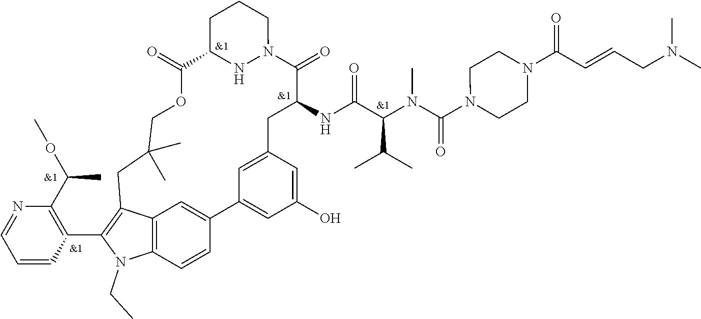 |
| BA 264 | 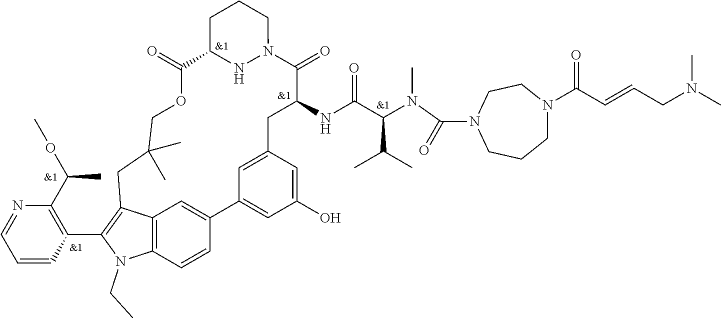 |
| BA 265 | 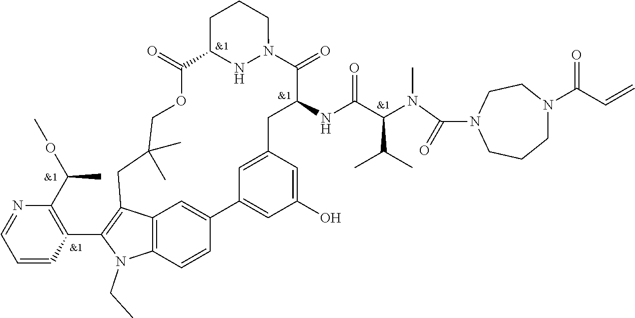 |
| BA 266 | 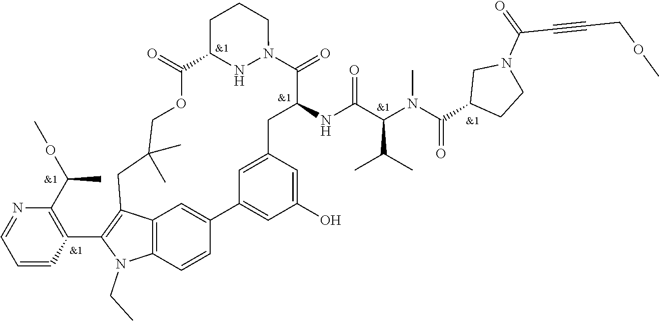 |
| BA 267 | 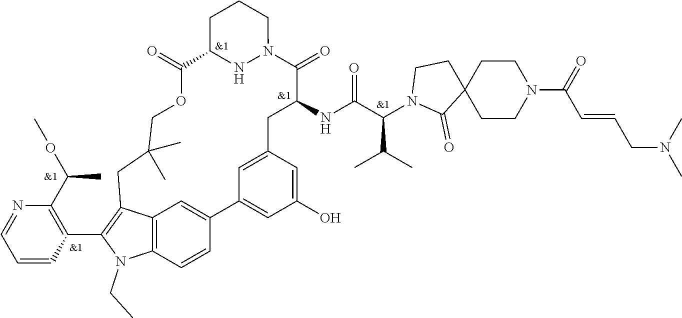 |
| BA 268 | 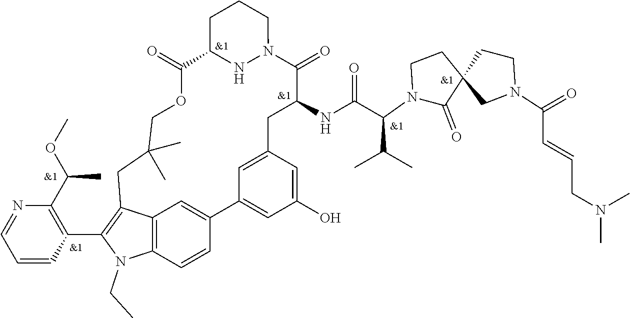 |
| BA 269 | 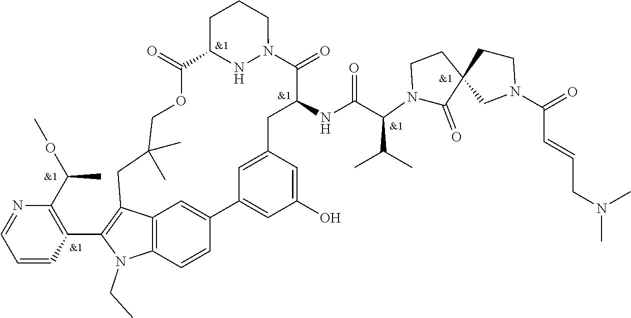 |
| BA 270 | 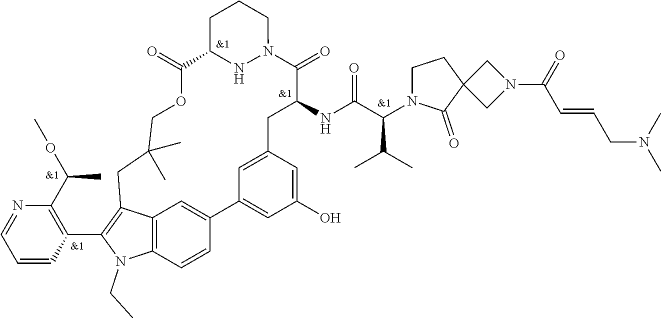 |
| BA 271 | 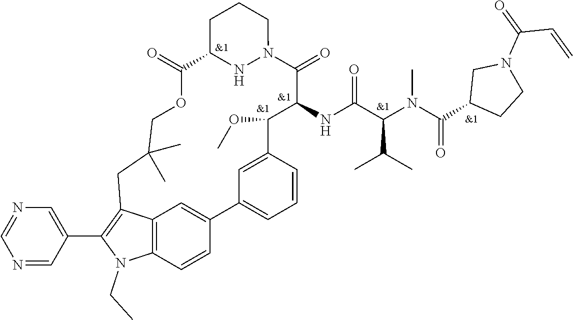 |
| BA 272 | 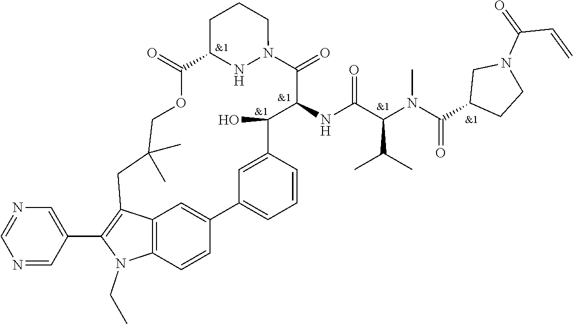 |
| BA 273 | 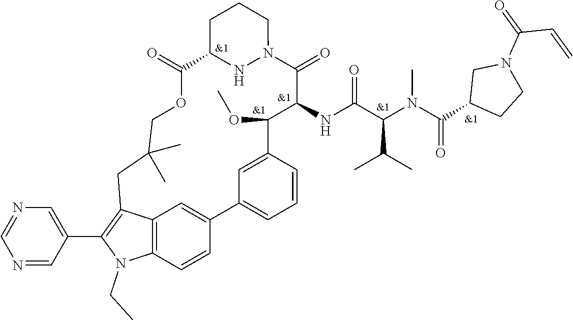 |
| BA 274 | 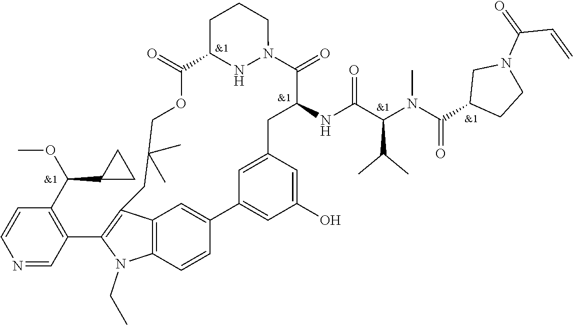 |
| BA 275 | 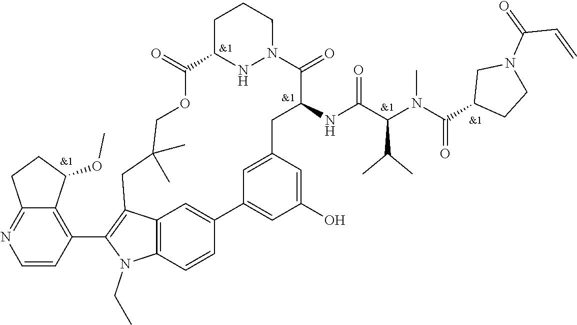 |
| BA 276 | 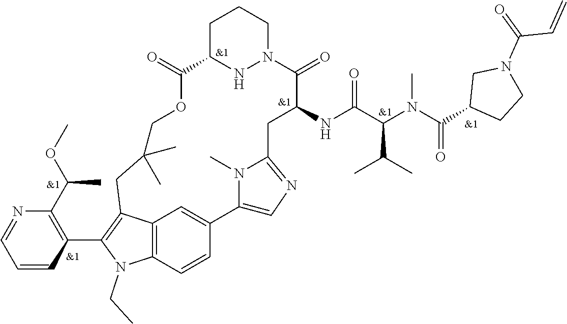 |
| BA 277 | 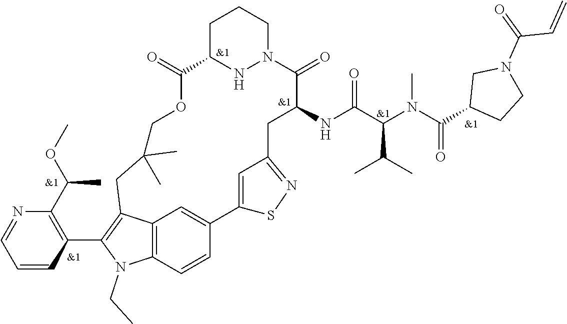 |
| BA 278 | 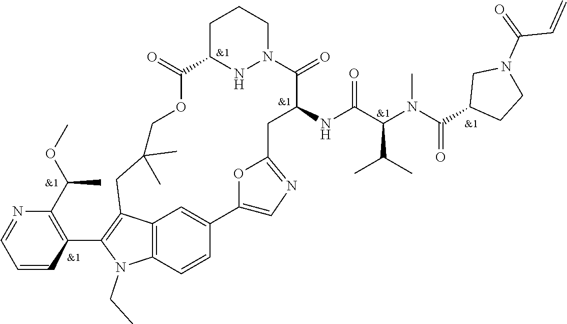 |
| BA 279 | 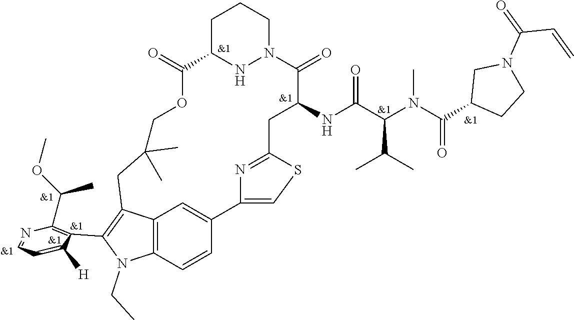 |
| BA 280 | 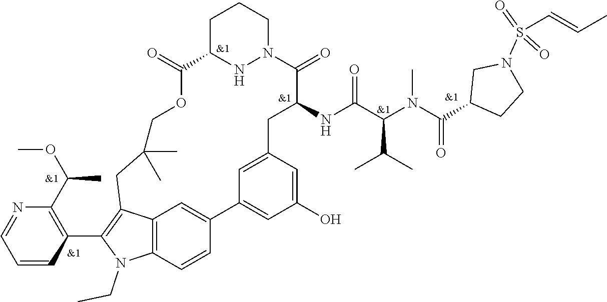 |
| BA 281 | 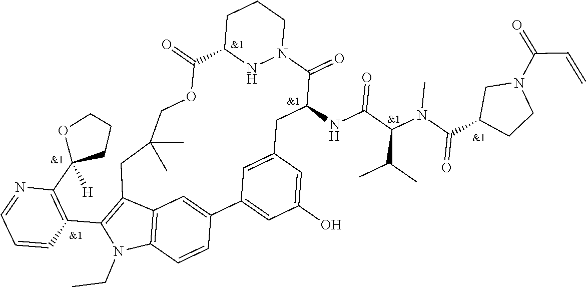 |
| BA 282 | 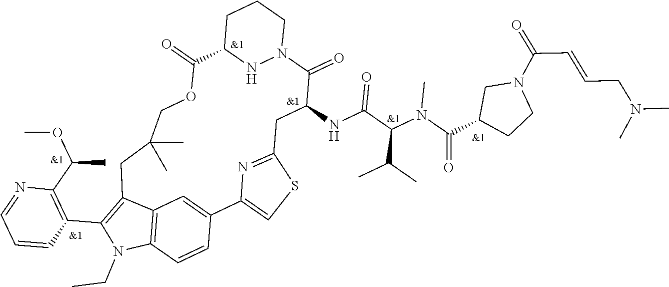 |
| BA 283 | 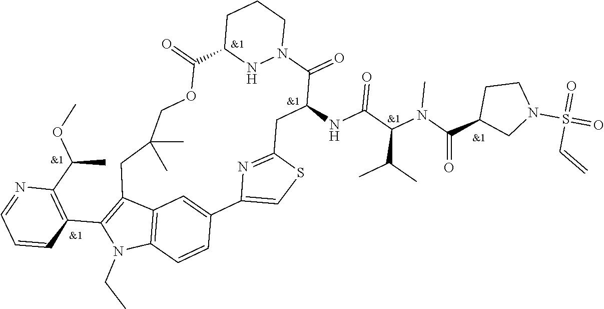 |
| BA 284 | 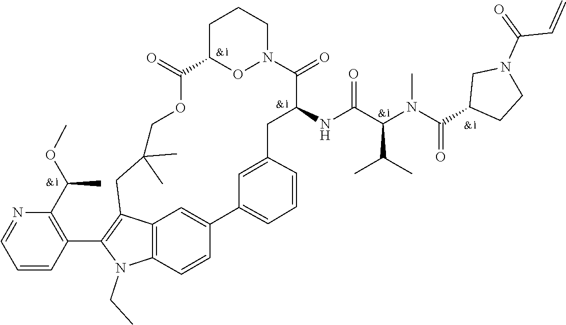 [SMILES: C=CC(=O)N1CC[C@H](C(=O)N(C)[C@H](C(=O)N[C@H]2Cc3cccc(c3)-c3ccc4c(c3)c(c(-c3cccnc3[C@H](C)OC)n4CC)CC(C)(C)COC(=O)[C@@H]3CCCN(O3)C2=O)C(C)C)C1] |
| BA 285 | 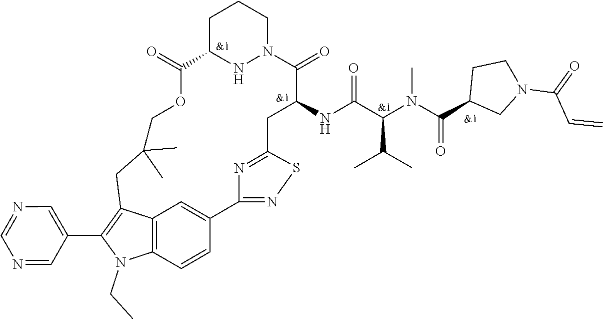 [SMILES: C=CC(=O)N1CC[C@H](C(=O)N(C)[C@H](C(=O)N[C@H]2Cc3nc(ns3)-c3ccc4c(c3)c(c(-c3cncnc3)n4CC)CC(C)(C)COC(=O)[C@@H]3CCCN(N3)C2=O)C(C)C)C1] |
| BA 286 | 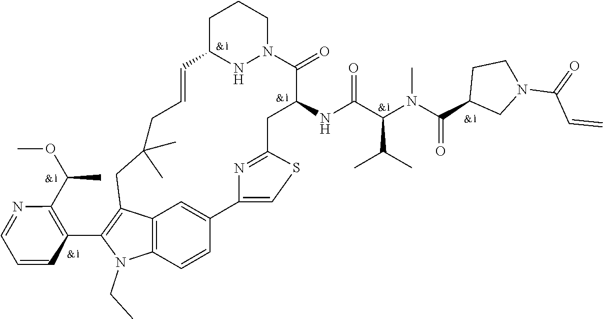 |
| BA 287 | 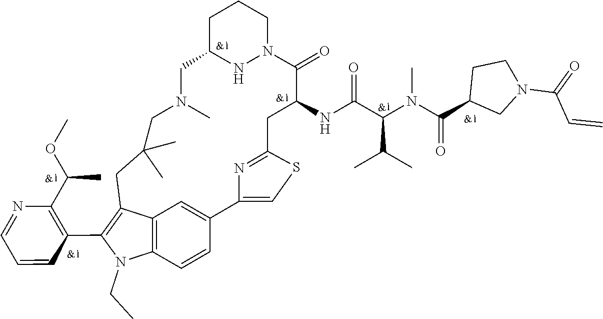 |
| BA 288 | 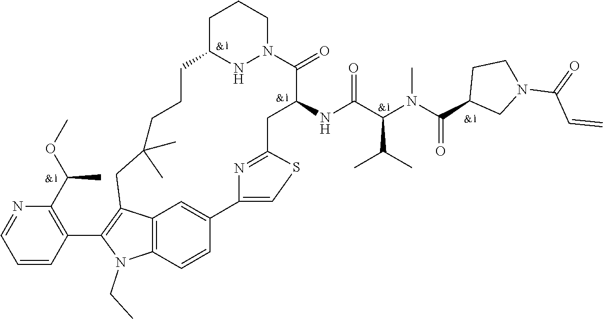 [SMILES: C=CC(=O)N1CC[C@H](C(=O)N(C)[C@H](C(=O)N[C@H]2Cc3nc(cs3)-c3ccc4c(c3)c(c(-c3cccnc3[C@H](C)OC)n4CC)CC(C)(C)CCC[C@@H]3CCCN(N3)C2=O)C(C)C)C1] |
| BA 289 | 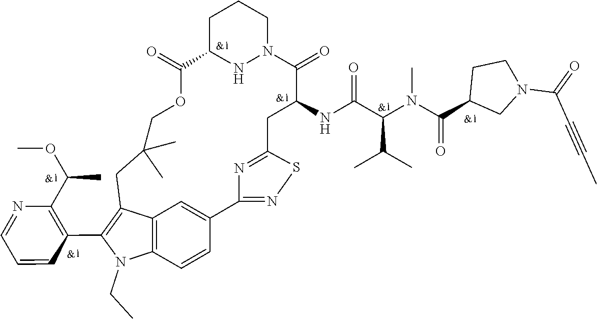 |
| BA 290 | 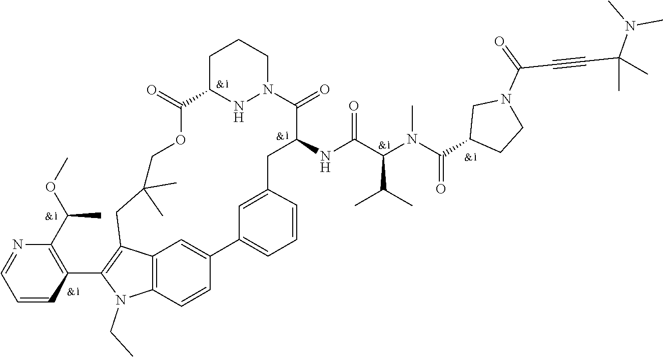 |
| BA 291 | 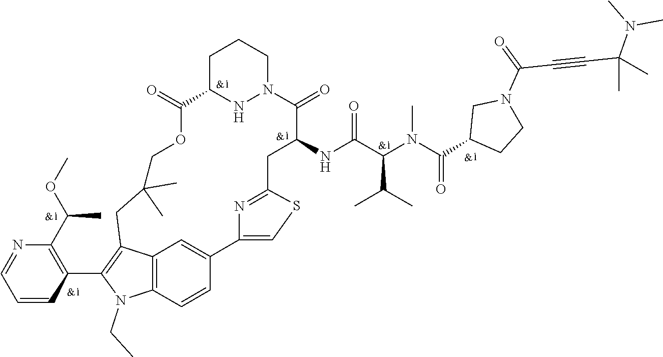 |
| BA 292 | 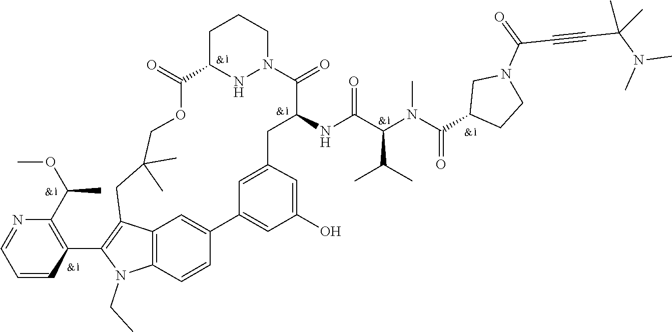 |
| BA 293 | 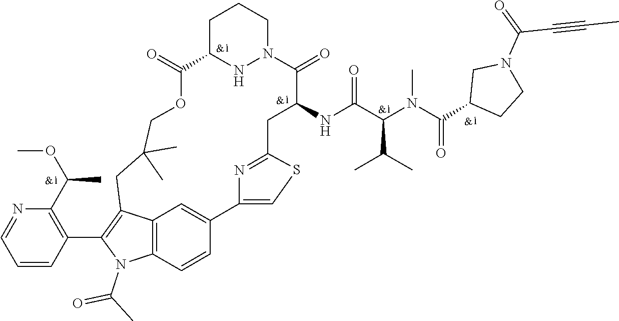 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(C)=O)CC(C)(C)COC(=O)[C@@H]3CCCN(N3)C2=O)C(C)C)C1] |
| BA 294 | 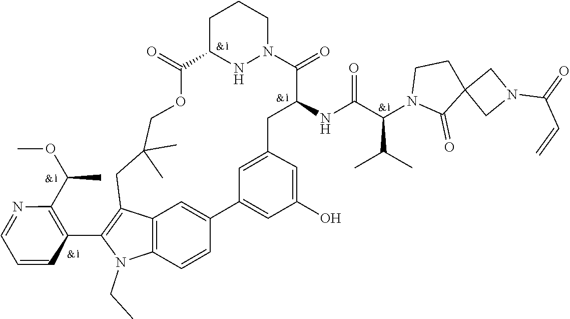 |
| BA 295 | 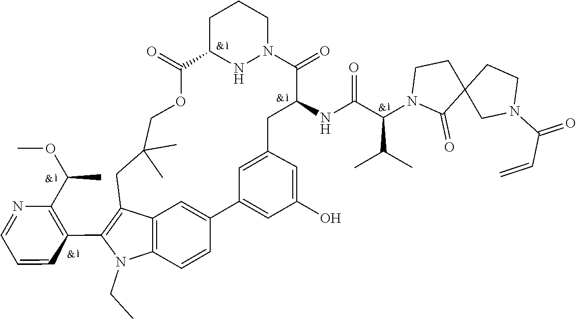 |
| BA 296 | 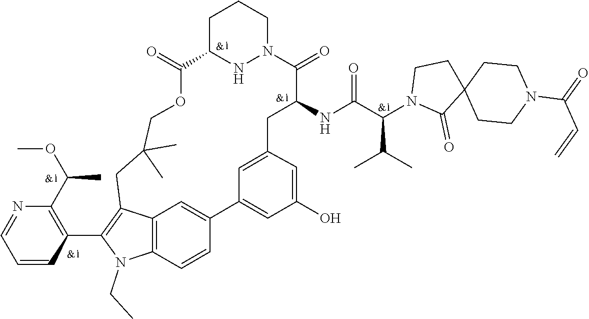 |
| BA 297 | 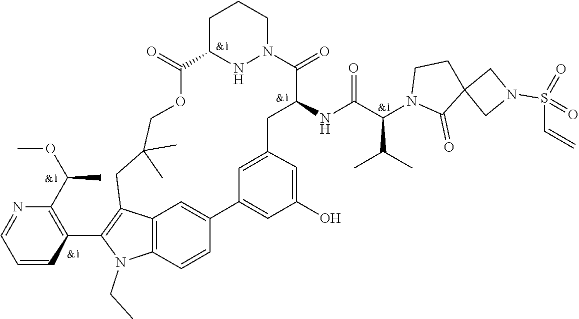 |
| BA 298 | 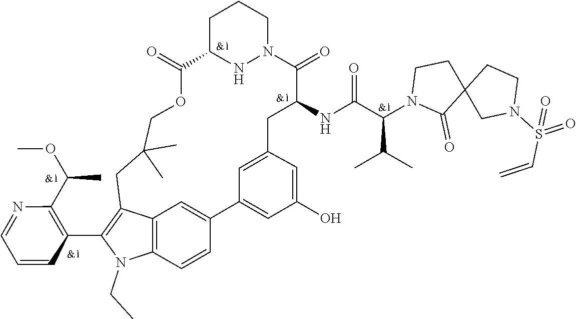 |
| BA 299 | 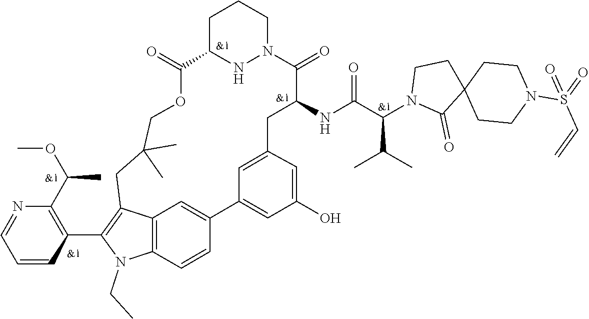 |
| BA 300 | 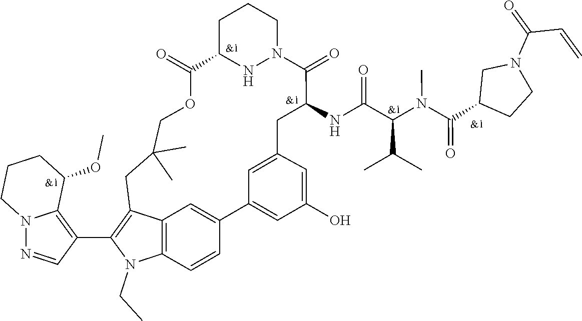 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CCC5)n4CC)CC(C)(C)COC(=O)[C@@H]3CCCN(N3)C2=O)C(C)C)C1] |
| BA 301 | 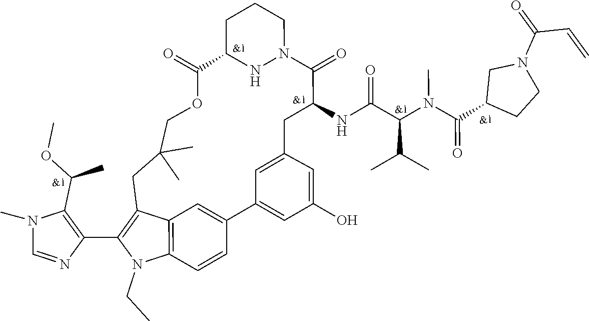 [SMILES: C=CC(=O)N1CC[C@H](C(=O)N(C)[C@H](C(=O)N[C@H]2Cc3cc(O)cc(c3)-c3ccc4c(c3)c(c(-c3ncn(C)c3[C@H](C)OC)n4CC)CC(C)(C)COC(=O)[C@@H]3CCCN(N3)C2=O)C(C)C)C1] |
| BA 302 | 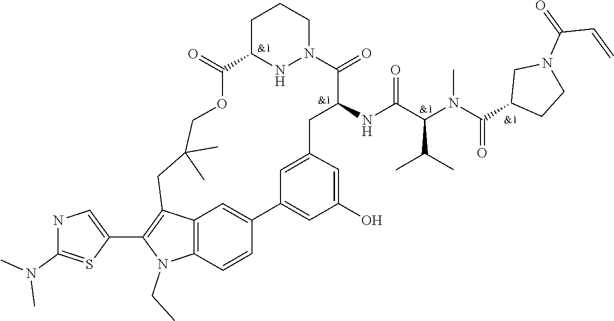 |
| BA 303 | 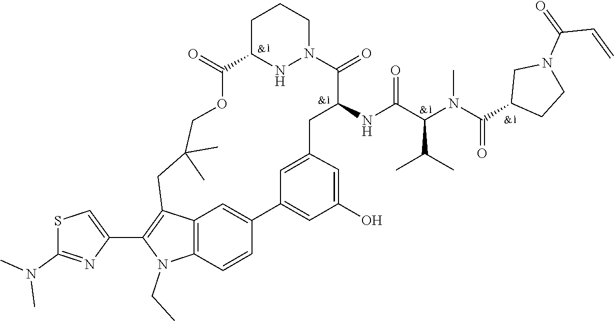 [SMILES: C=CC(=O)N1CC[C@H](C(=O)N(C)[C@H](C(=O)N[C@H]2Cc3cc(O)cc(c3)-c3ccc4c(c3)c(c(-c3csc(N(C)C)n3)n4CC)CC(C)(C)COC(=O)[C@@H]3CCCN(N3)C2=O)C(C)C)C1] |
| BA 304 | 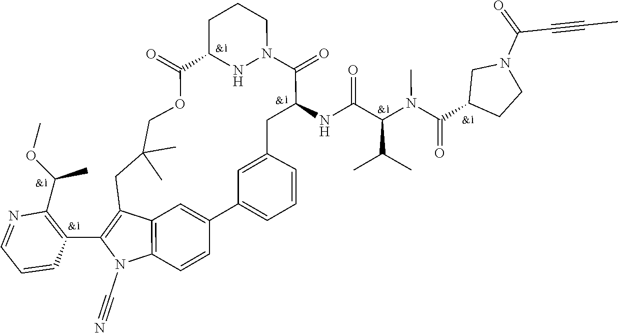 |
| BA 305 | 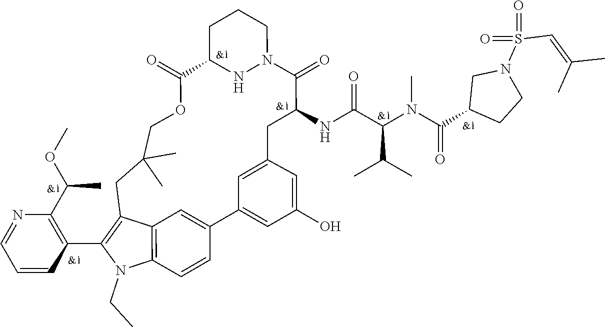 |
| BA 306 | 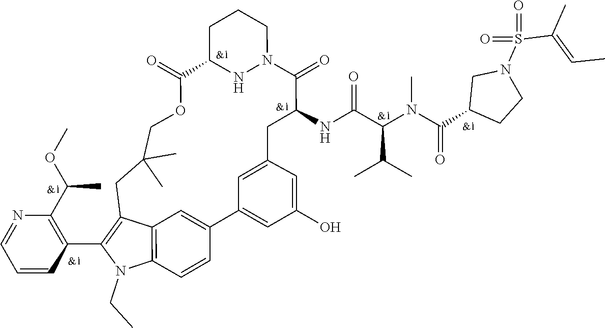 |
| BA 307 | 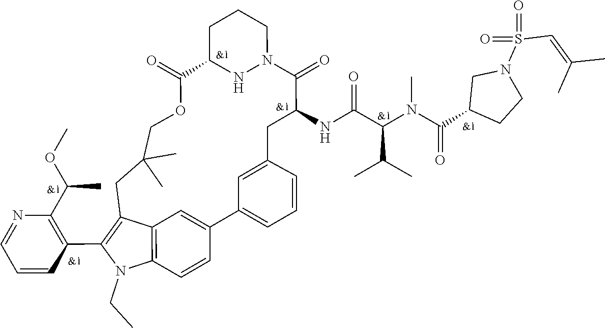 |
| BA 308 | 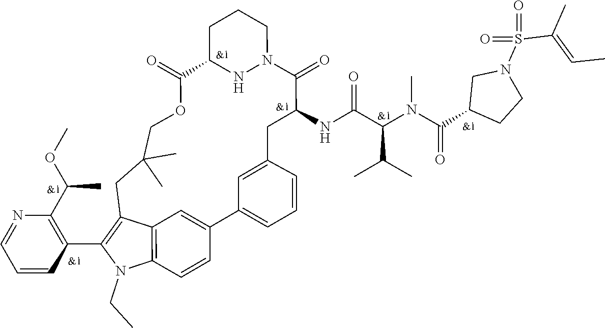 |
| BA 309 | 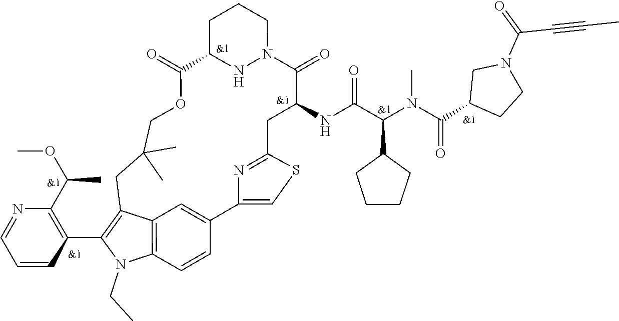 |
| BA 310 | 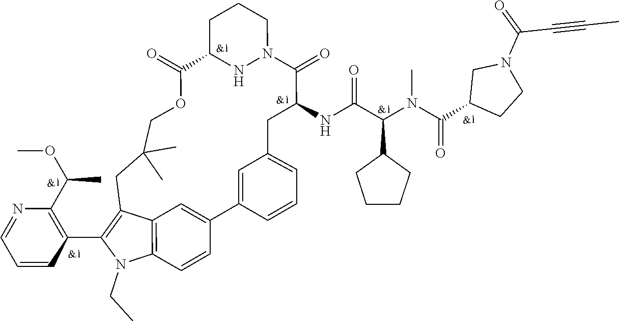 |
| BA 311 | 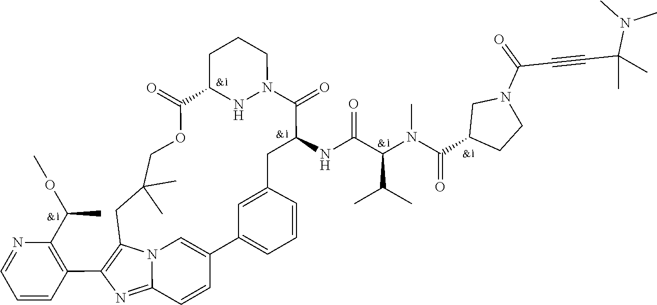 [SMILES: CO[C@@H](C)c1ncccc1-c1nc2ccc3cn2c1CC(C)(C)COC(=O)[C@@H]1CCCN(N1)C(=O)[C@@H](NC(=O)[C@H](C(C)C)N(C)C(=O)[C@H]1CCN(C(=O)C#CC(C)(C)N(C)C)C1)Cc1cccc-3c1] |
| BA 312 | 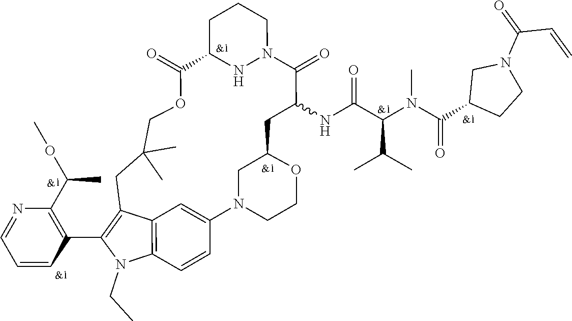 |
| BA 313 | 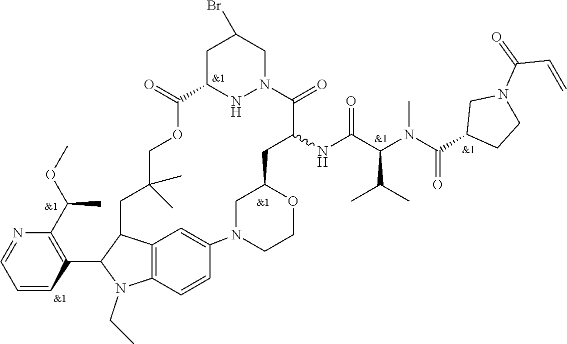 |
| BA 314 | 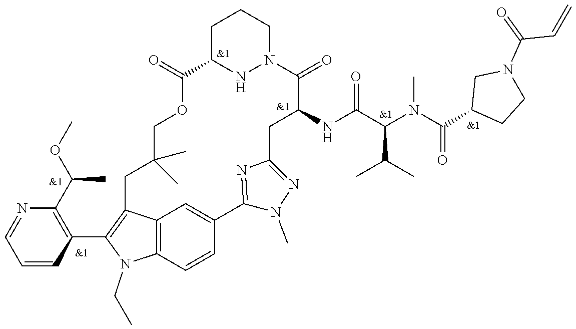 |
| BA 316 | 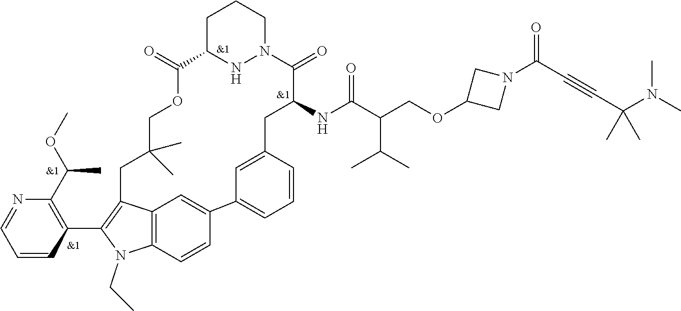 |
| BA 317 | 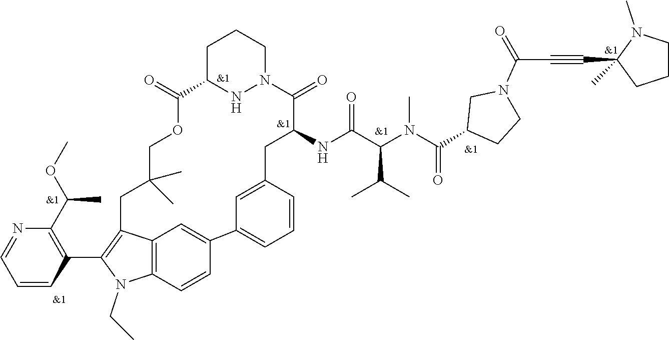 |
| BA 318 | 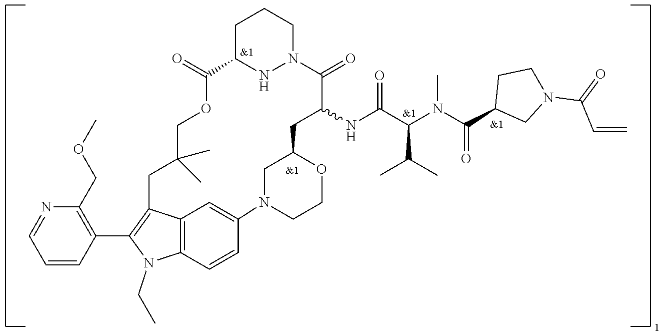 |
| BA 319 | 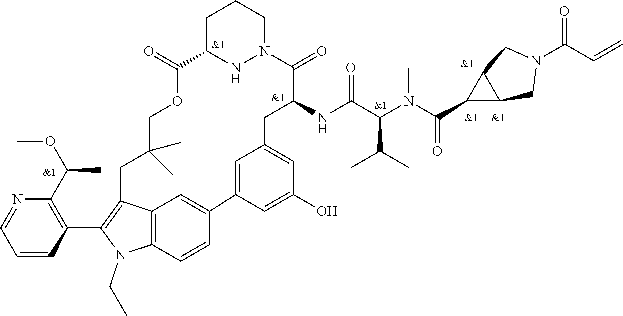 |
| BA 320 | 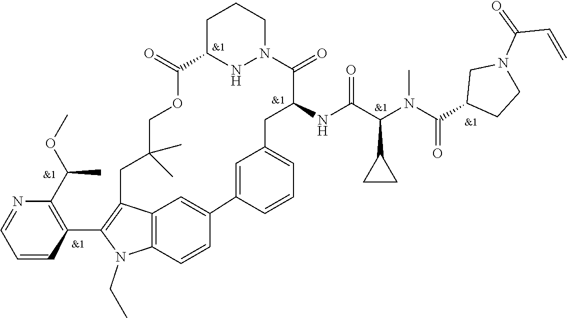 |
| BA 321 | 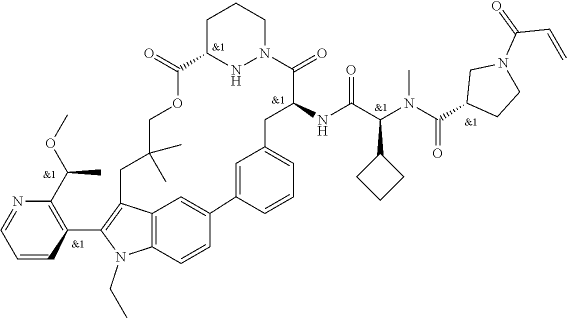 |
| BA 322 | 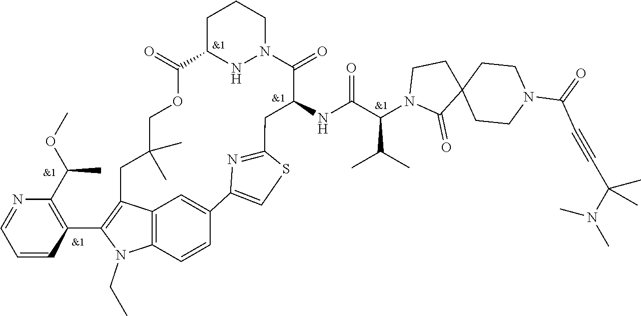 |
| BA 323 | 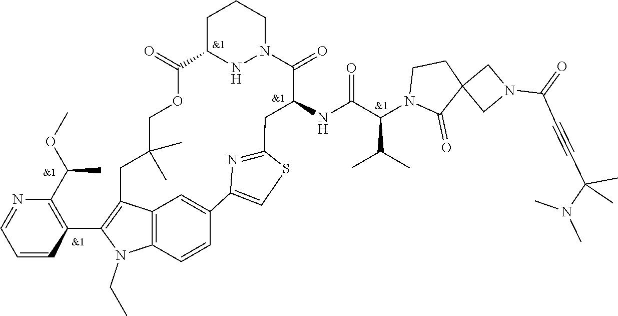 |
| BA 324 | 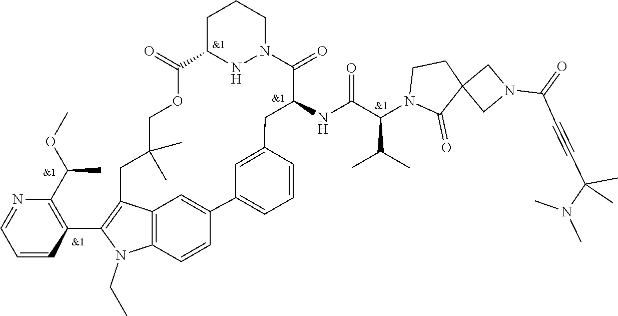 |
| BA 325 | 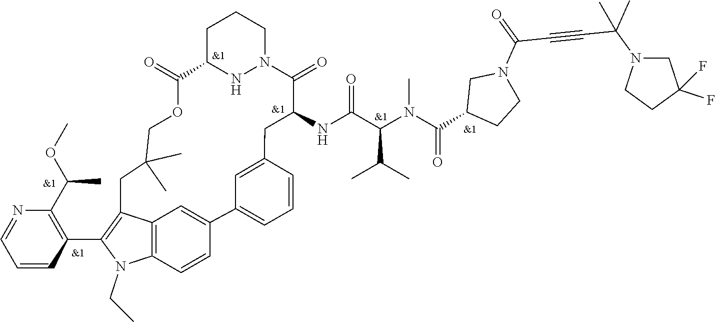 |
| BA 326 | 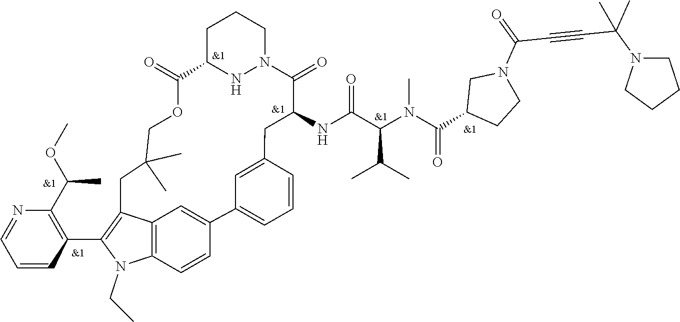 |
| BA 327 | 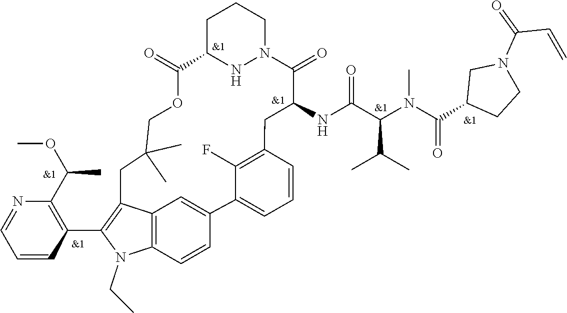 |
| BA 328 | 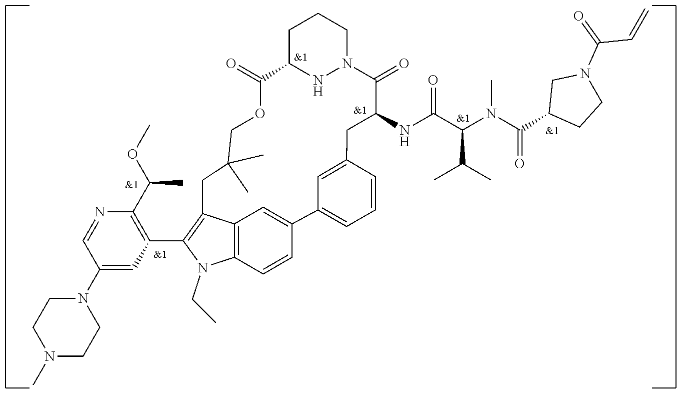 |
| BA 329 | 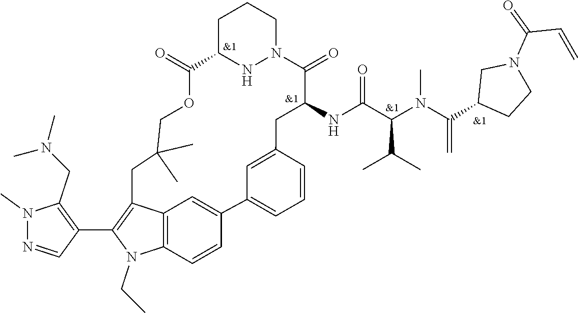 [SMILES: C=CC(=O)N1CC[C@H](C(=C)N(C)[C@H](C(=O)N[C@H]2Cc3cccc(c3)-c3ccc4c(c3)c(c(-c3cnn(C)c3CN(C)C)n4CC)CC(C)(C)COC(=O)[C@@H]3CCCN(N3)C2=O)C(C)C)C1] |
| BA 330 | 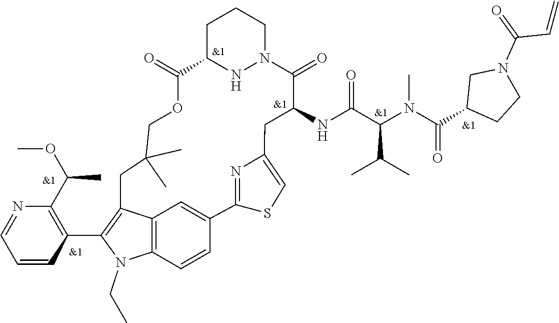 |
| BA 331 | 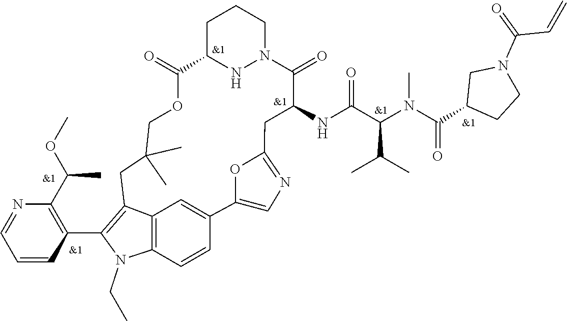 |
| BA 332 | 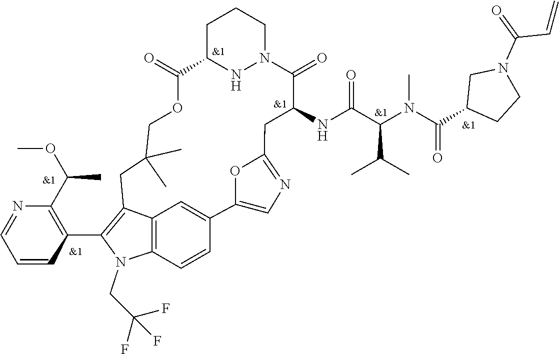 |
| BA 333 | 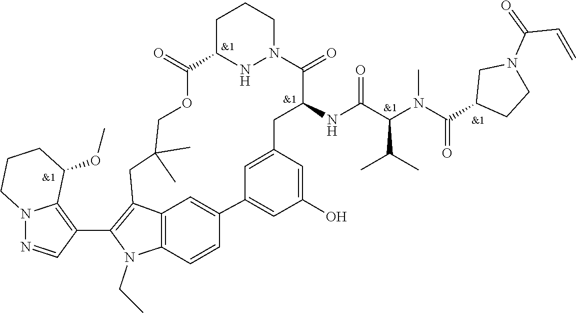 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CCC5)n4CC)CC(C)(C)COC(=O)[C@@H]3CCCN(N3)C2=O)C(C)C)C1] |
| BA 334 | 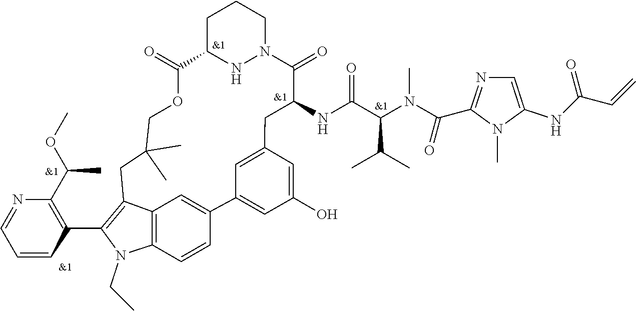 |
| BA 335 | 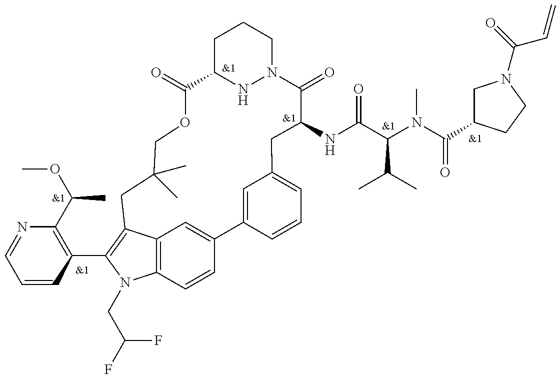 |
| BA 336 | 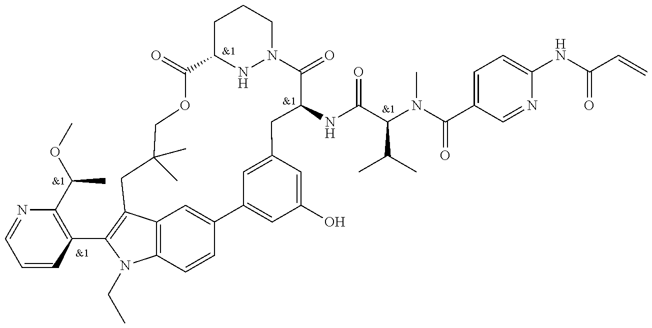 |
| BA 337 | 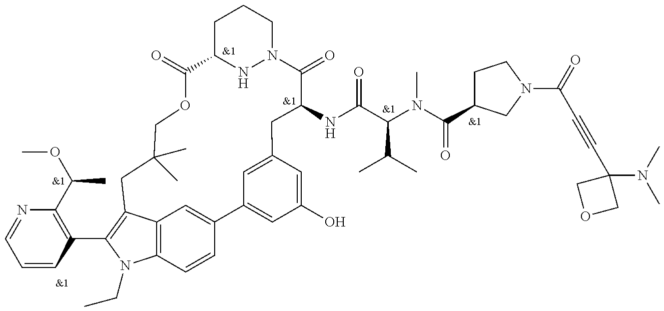 |
| BA 338 | 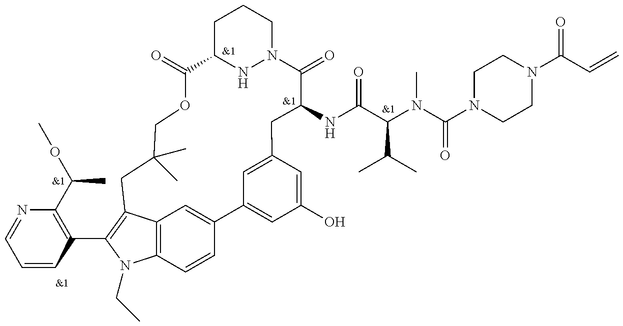 |
| BA 339 | 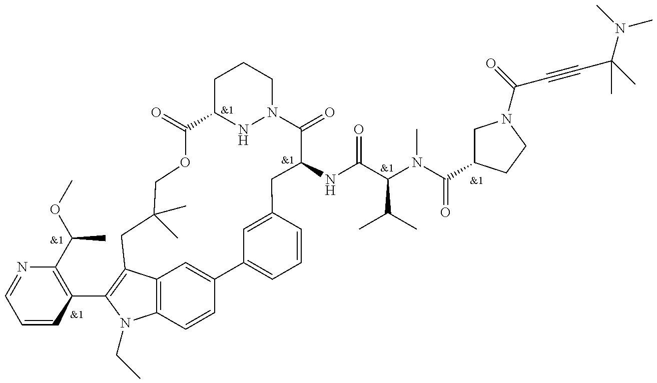 |
| BA 340 | 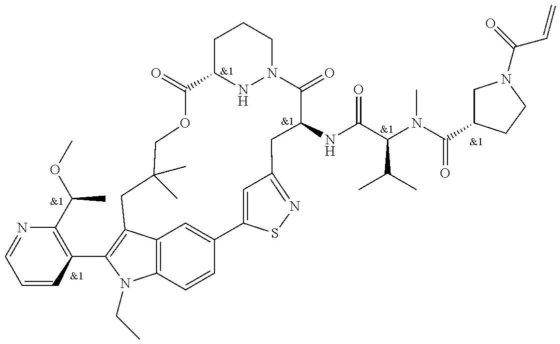 |
| BA 341 | 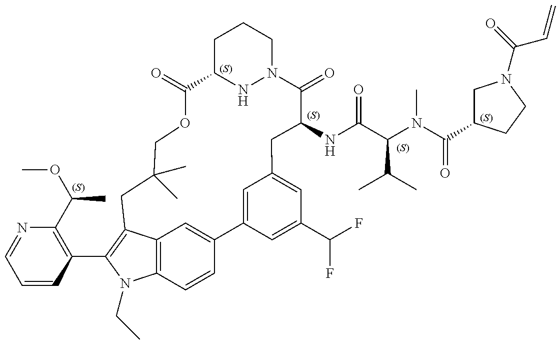 |
| BA 342 | 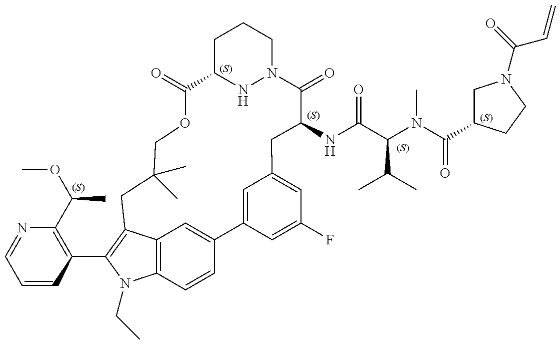 |
| BA 343 | 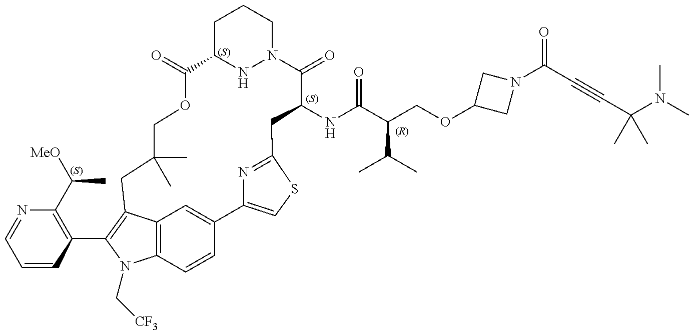 |
| BA 344 | 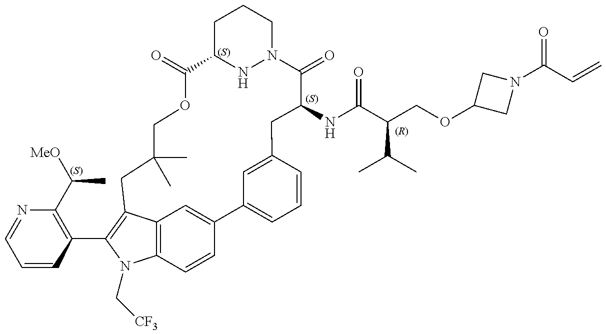 |
| BA 345 | 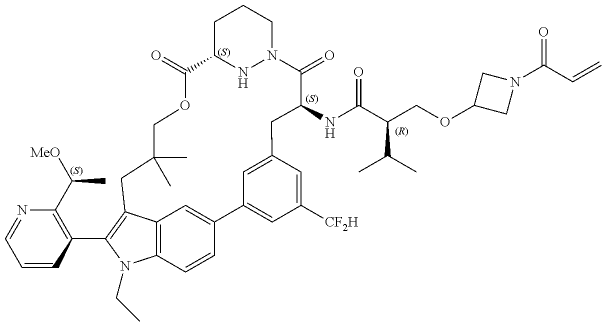 |
| BA 346 | 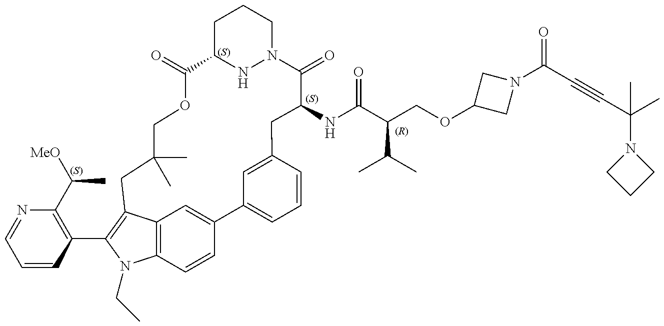 |
| BA 347 | 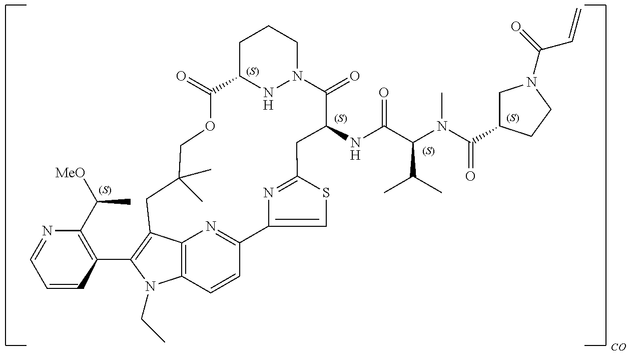 |
| BA 348 | 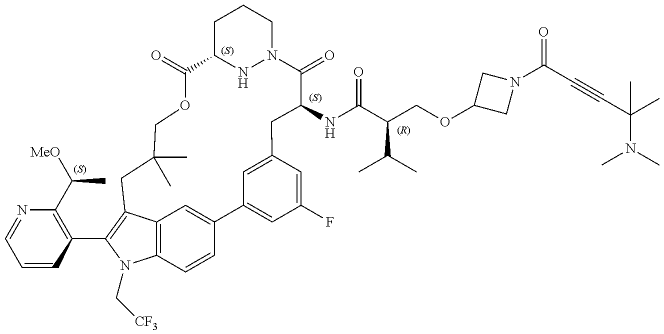 |
| BA 349 | 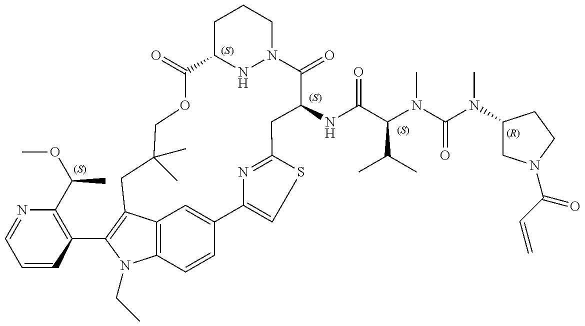 |
| BA 350 | 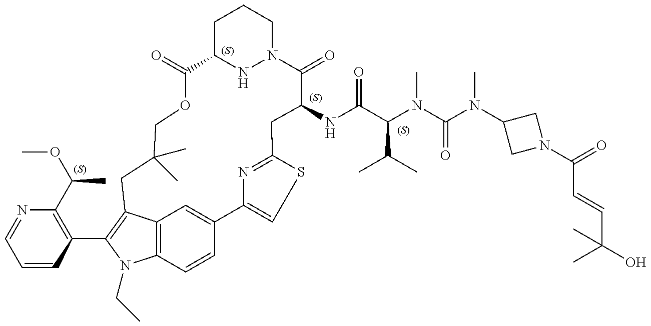 |
| BA 351 | 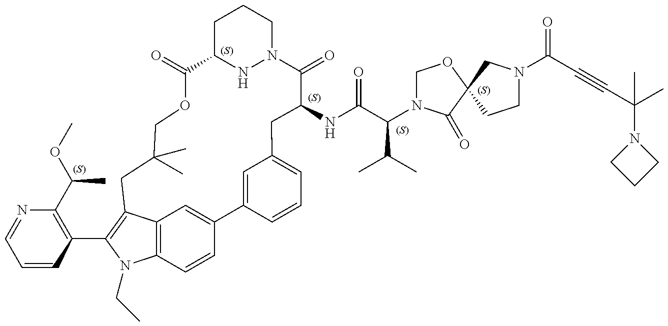 |
| BA 352 | 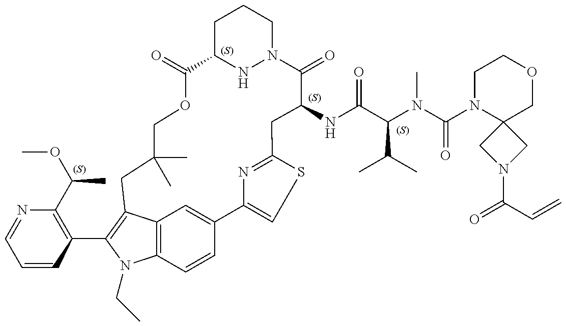 |
| BA 353 | 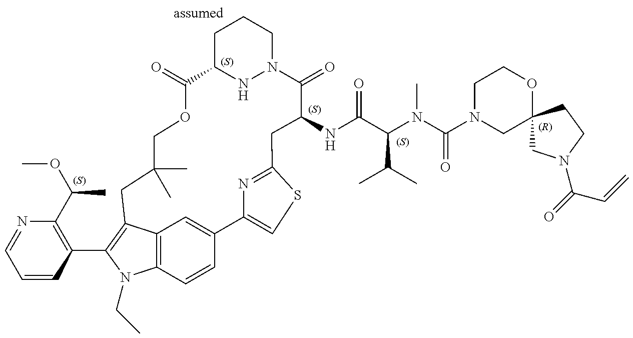 |
| BA 354 | 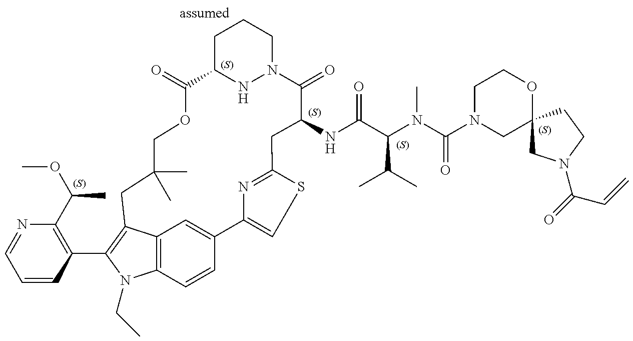 |
| BA 355 | 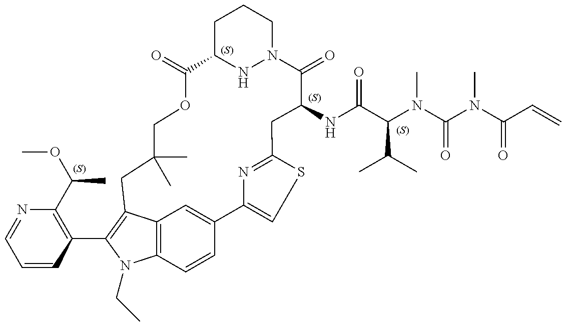 |
| BA 356 | 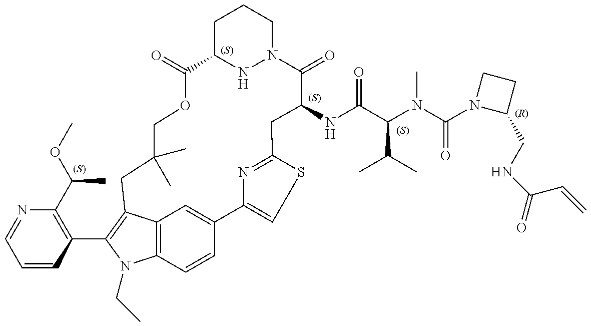 |
| BA 357 | 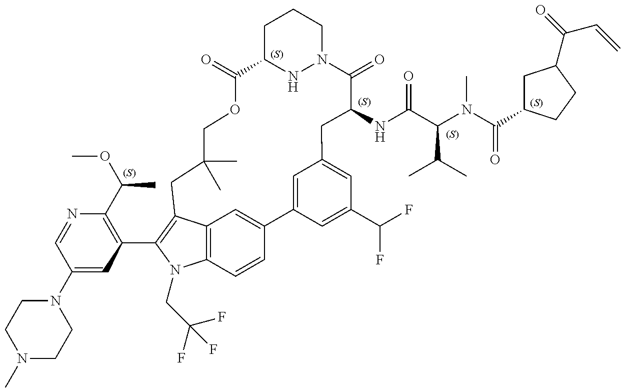 |
| BA 358 | 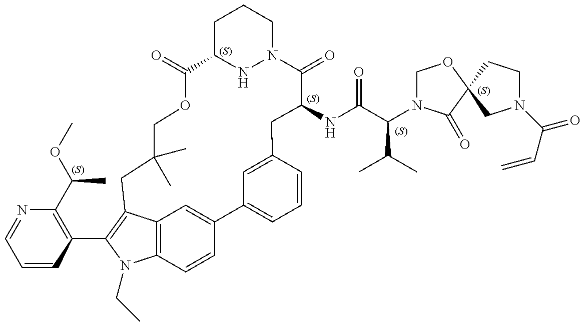 |
| BA 359 | 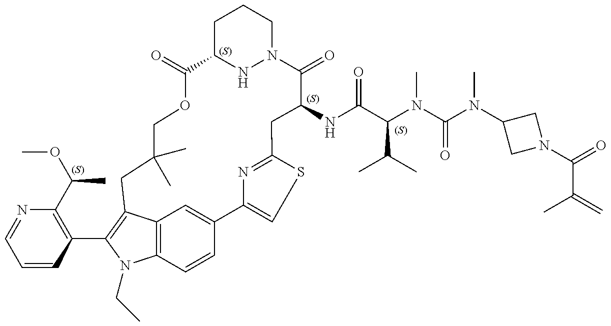 |
| BA 360 | 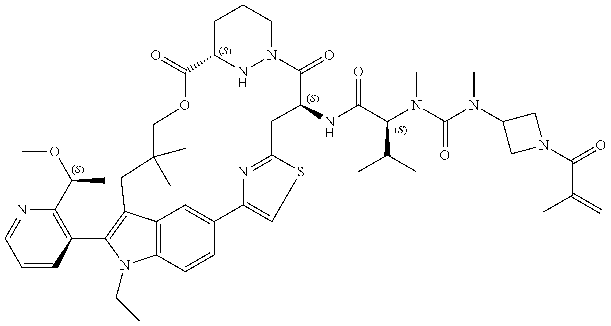 |
| BA 361 | 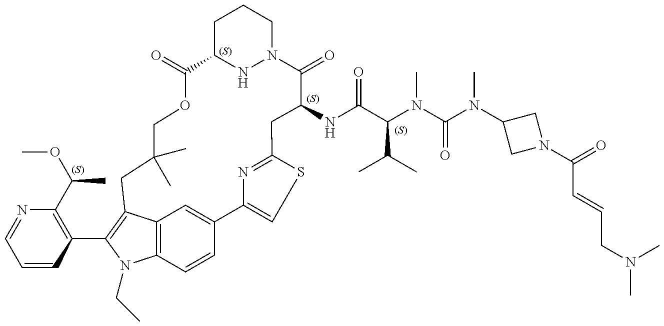 |
| BA 362 | 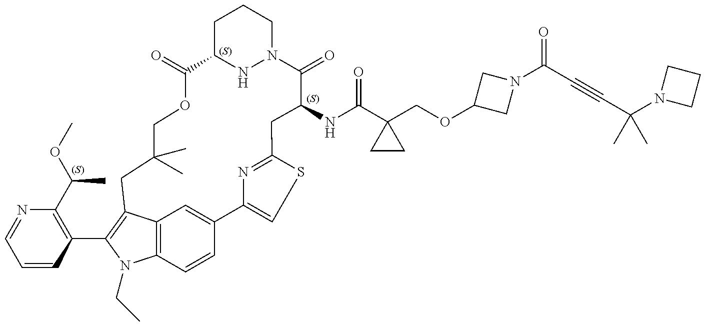 |
| BA 363 | 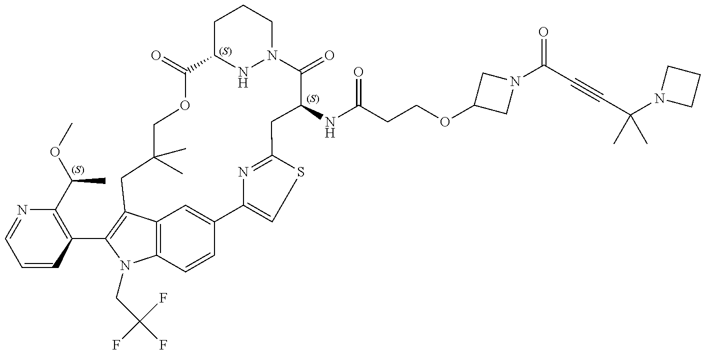 |
| BA 364 | 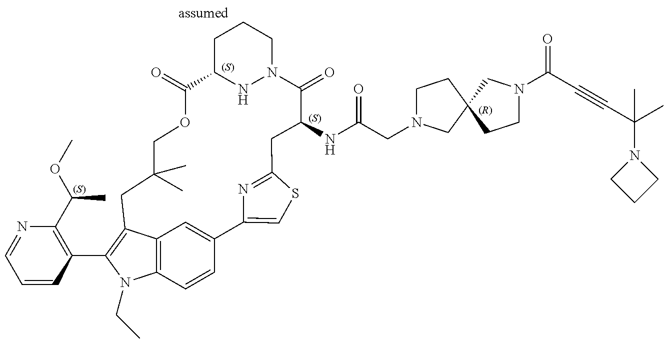 |
| BA 365 | 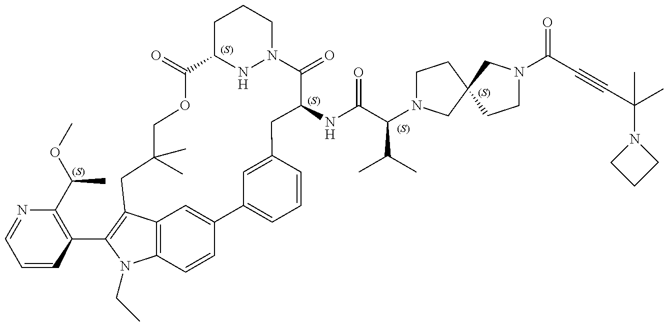 |
| BA 366 | 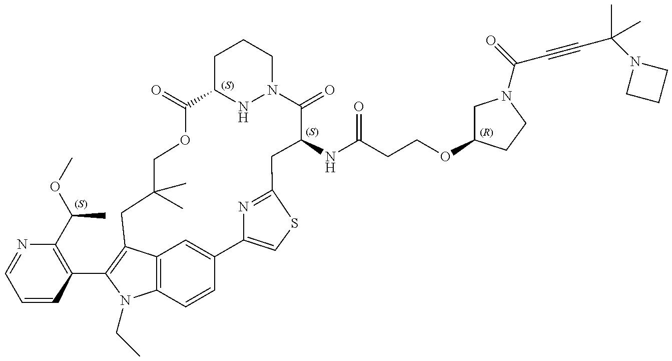 |
| BA 367 | 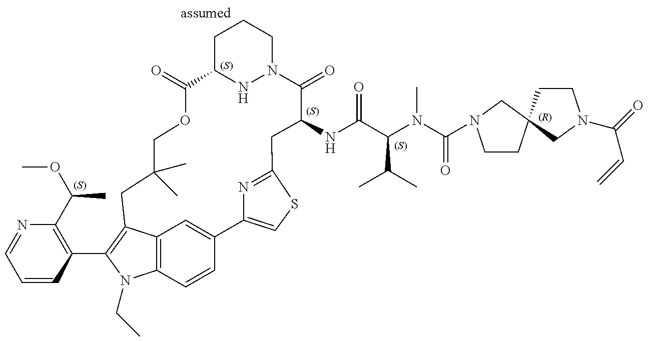 |
| BA 368 | 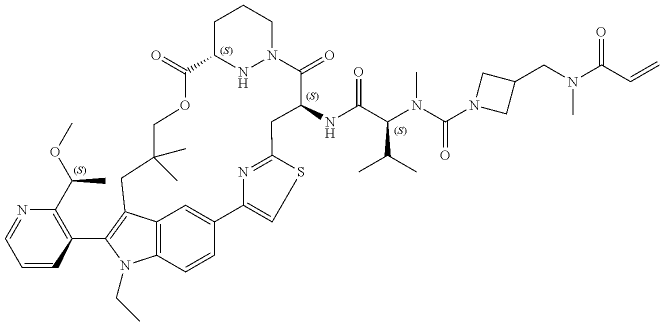 |
| BA 369 | 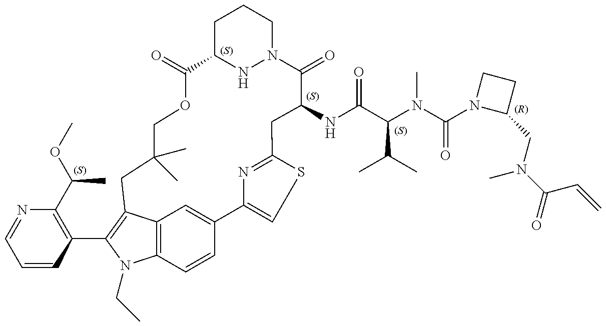 |
| BA 370 | 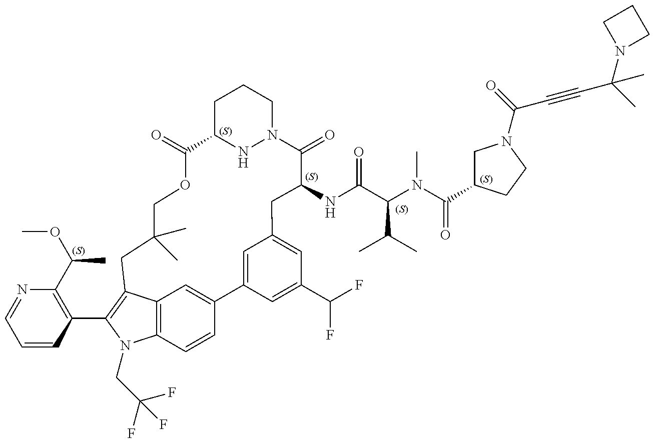 |
| BA 371 | 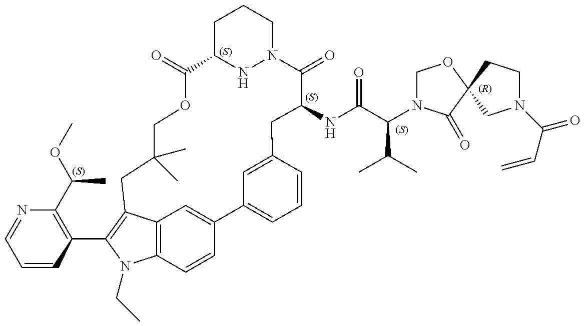 |
| BA 372 | 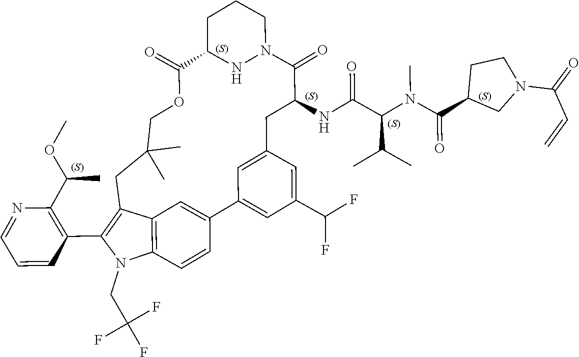 |
| BA 373 | 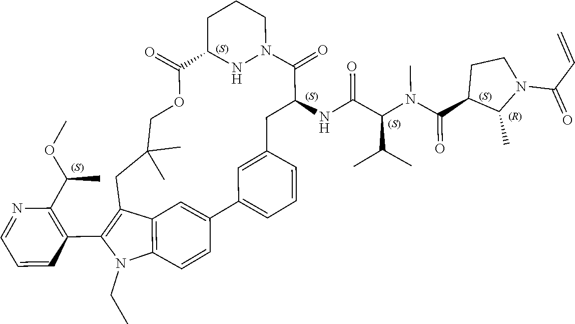 |
| BA 374 | 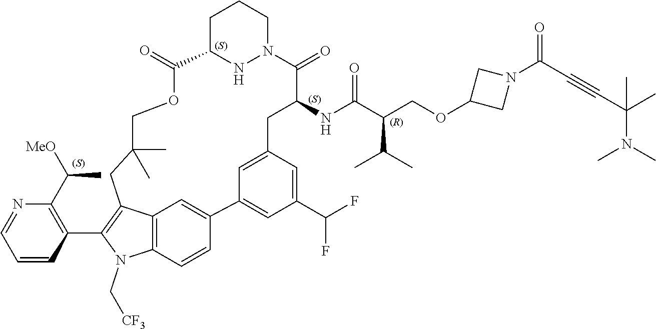 |
| BA 375 | 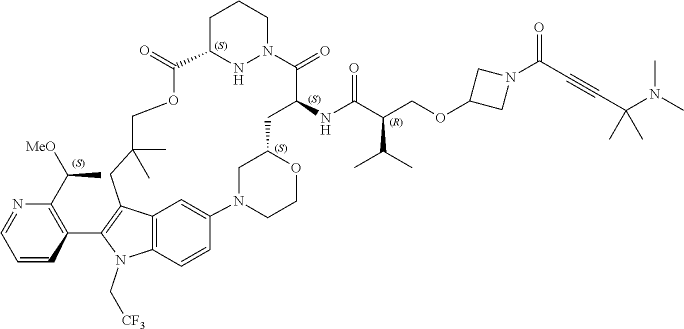 |
| BA 376 | 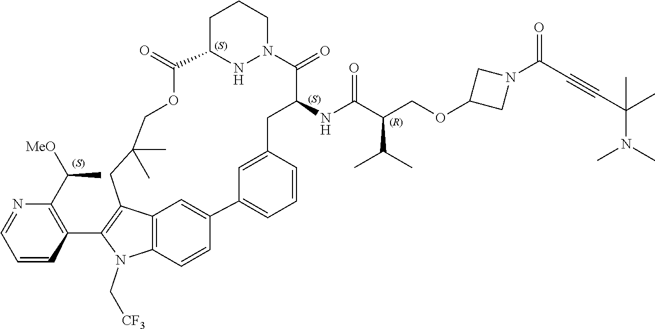 |
| BA 377 | 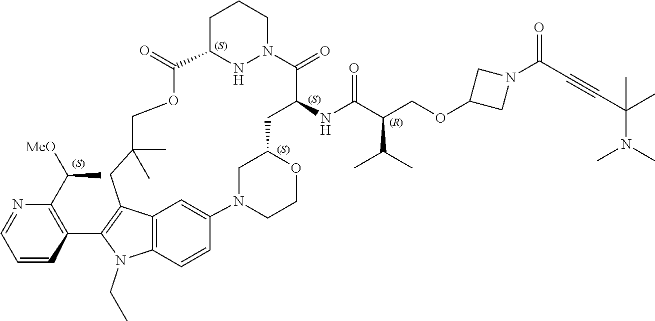 |
| BA 378 | 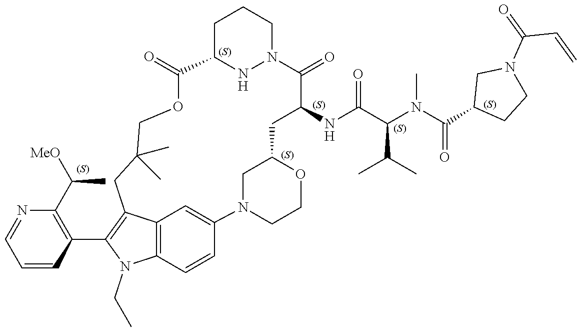 |
| BA 379 | 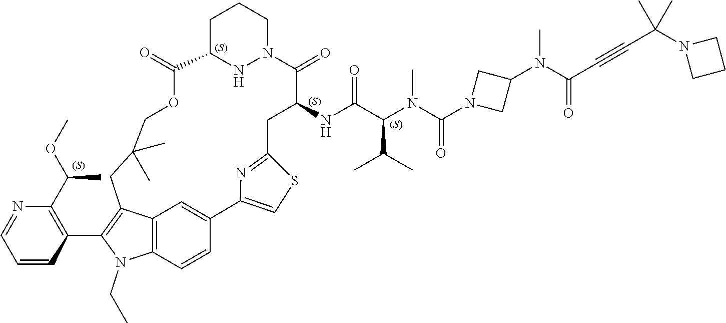 |
| BA 380 | 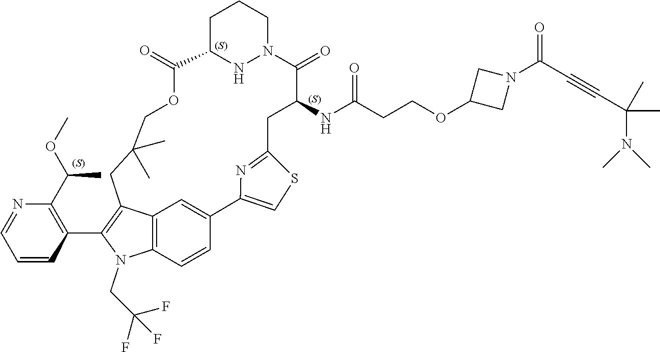 |
| BA 381 | 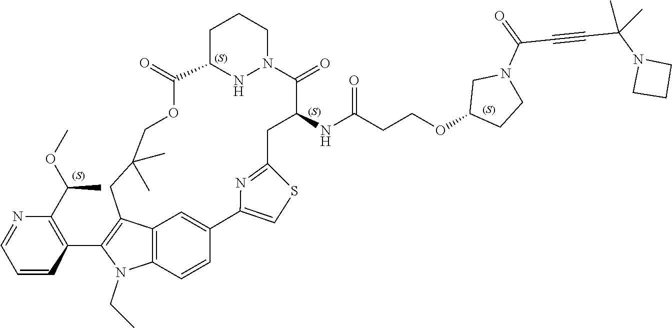 |
| BA 382 | 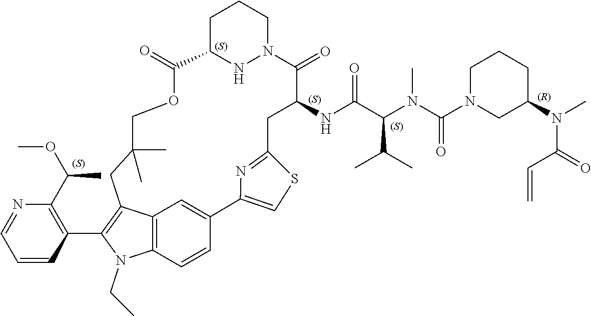 |
| BA 383 | 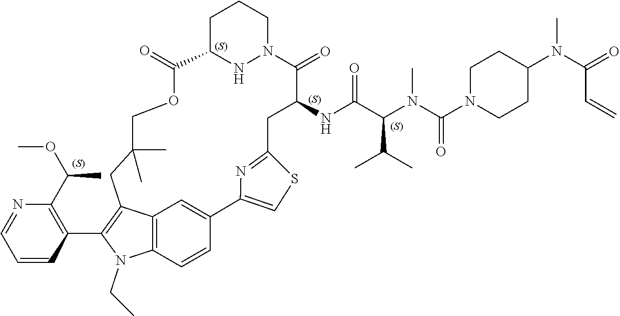 |
| BA 384 | 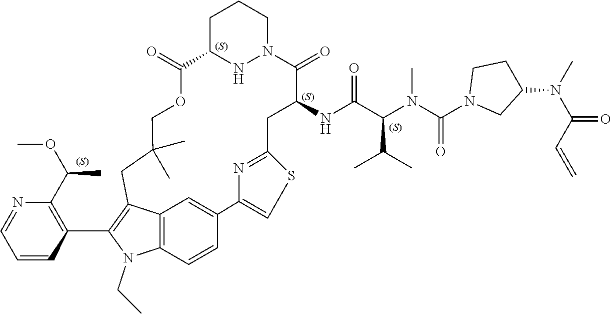 |
| BA 385 | 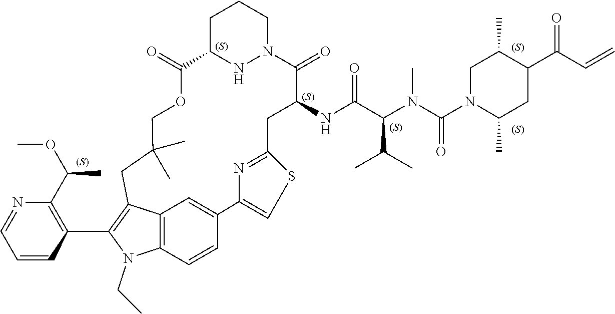 |
| BA 386 | 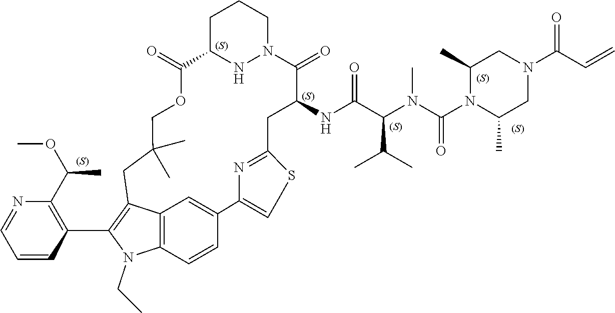 |
| BA 387 | 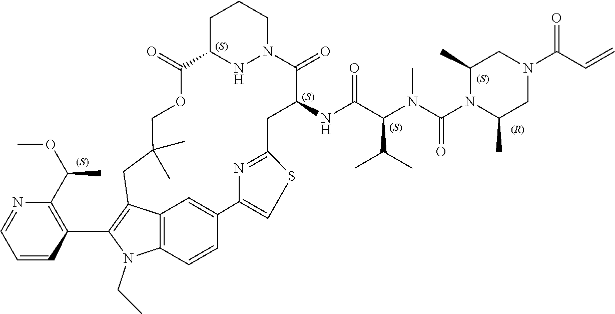 |
| BA 388 | 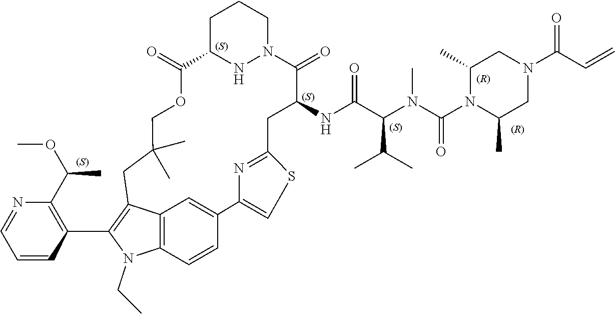 |
| BA 389 | 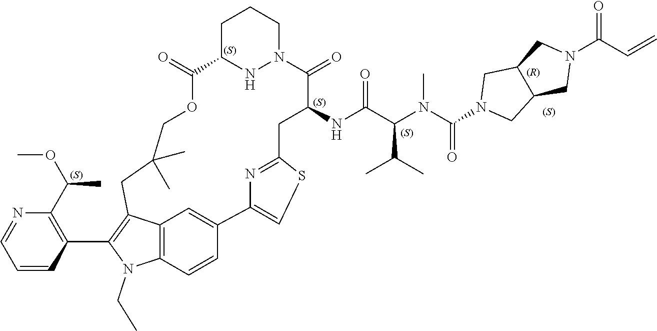 |
| BA 390 | 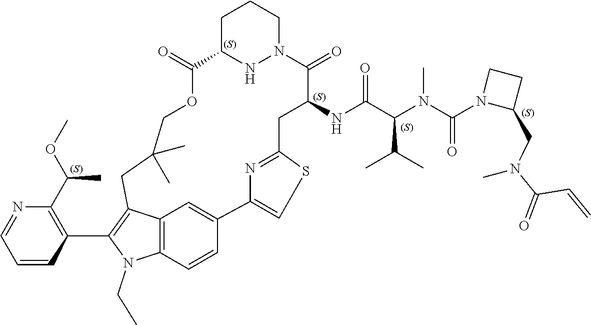 |
| BA 391 | 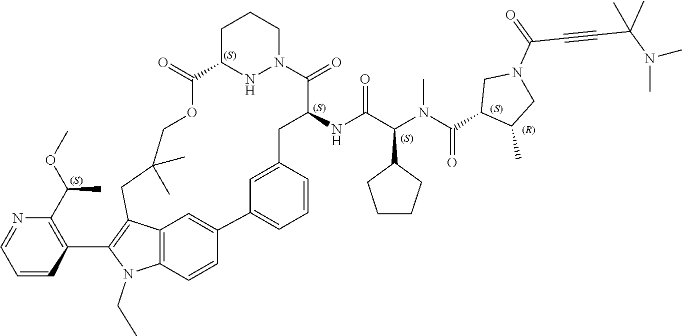 |
| BA 392 | 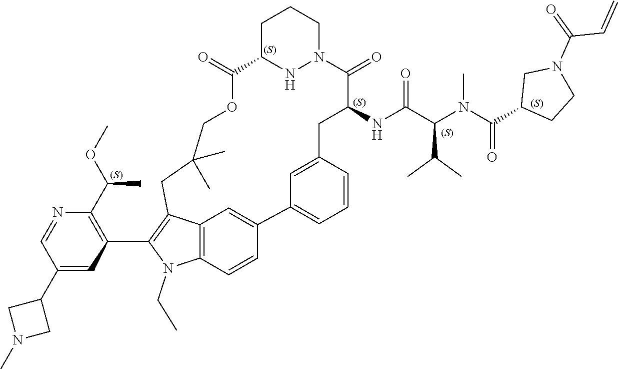 |
| BA 393 | 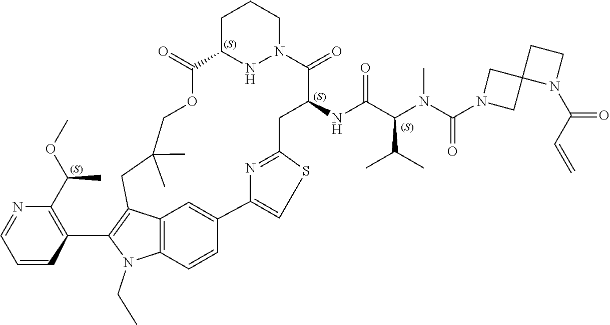 |
| BA 394 | 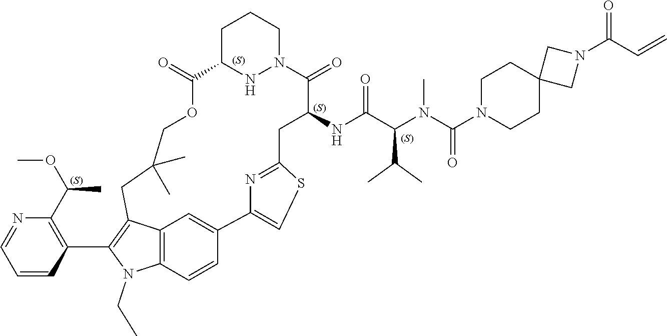 |
| BA 395 | 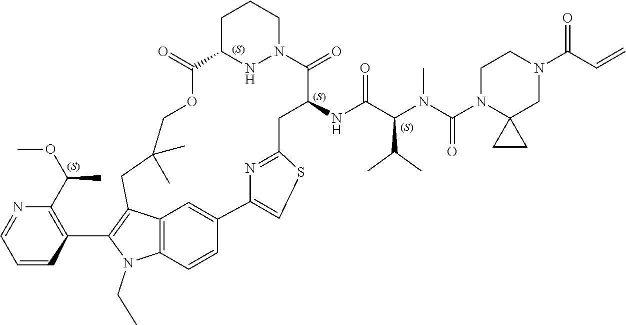 |
| BA 396 | 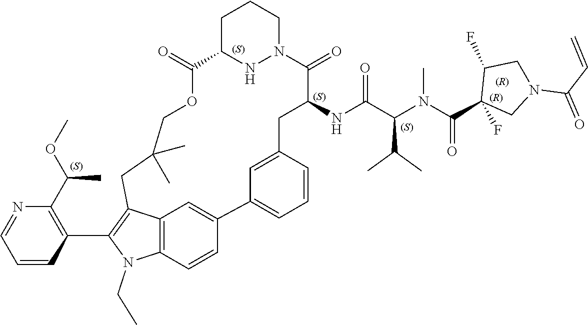 |
| BA 397 | 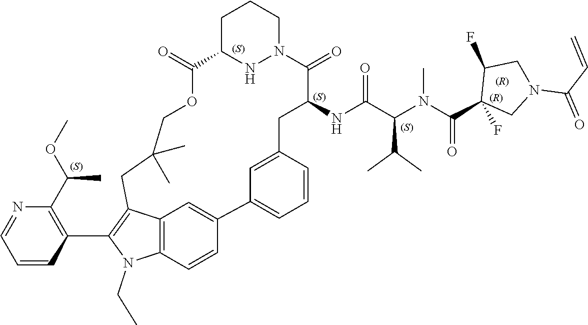 |
| BA 398 | 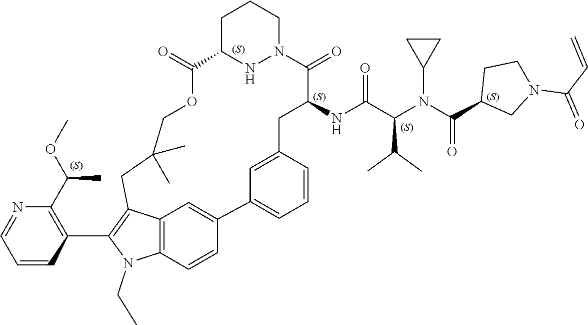 |
| BA 399 | 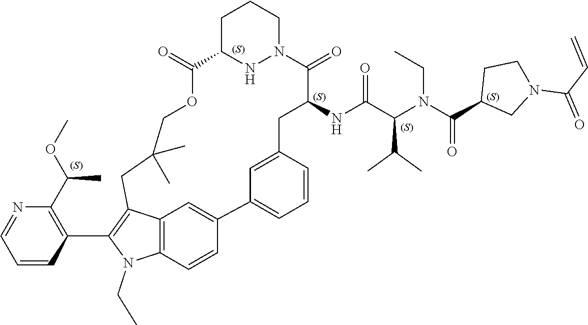 |
| BA 400 | 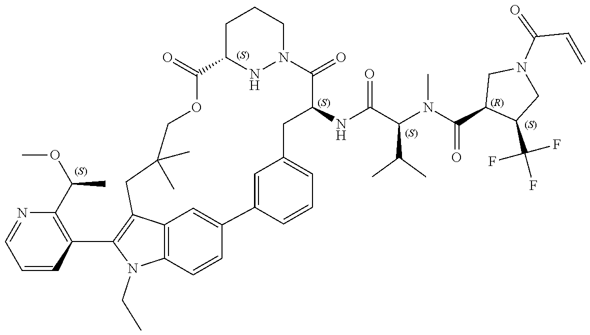 |
| BA 401 | 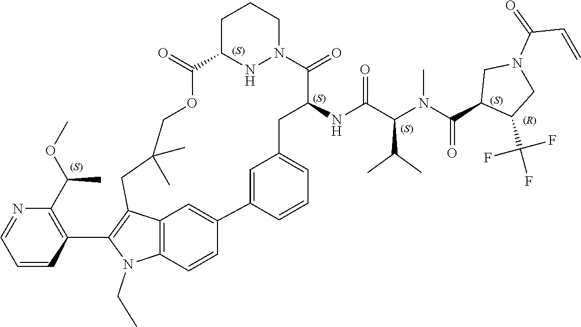 |
| BA 402 | 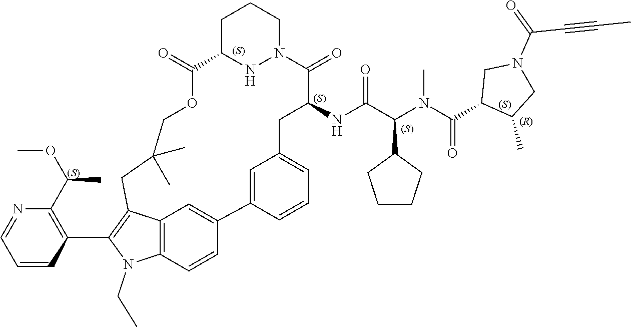 |
| BA 403 | 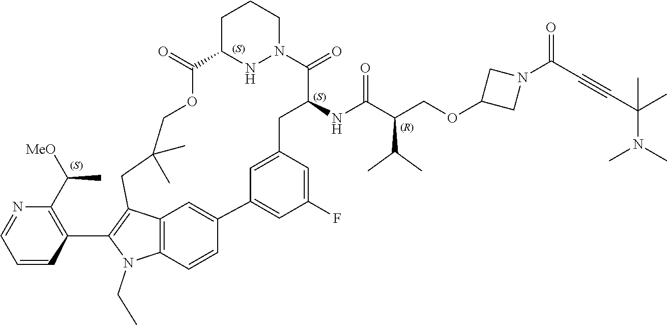 |
| BA 404 | 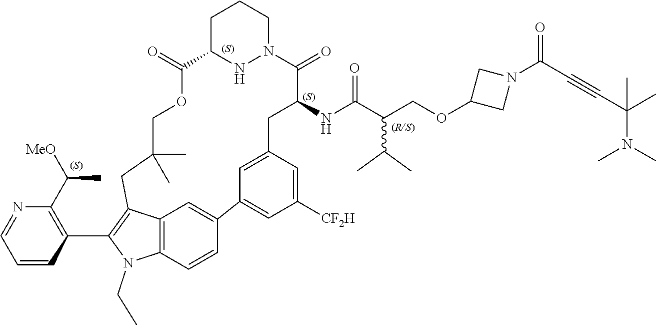 |
| BA 405 | 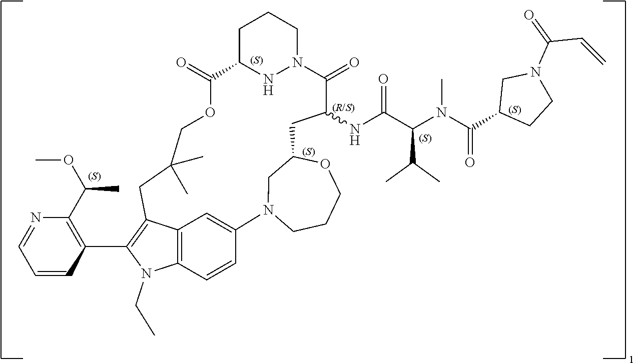 |
| BA 406 | 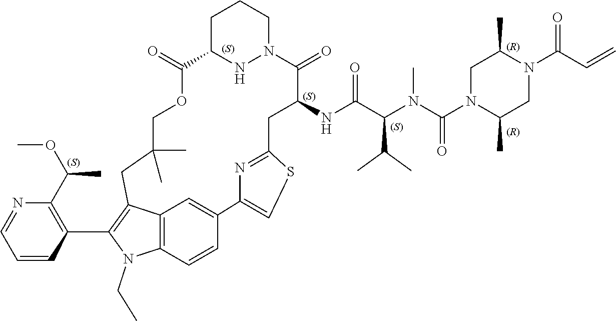 |
| BA 407 | 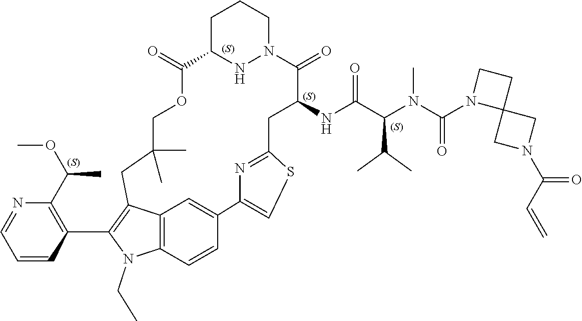 |
| BA 408 | 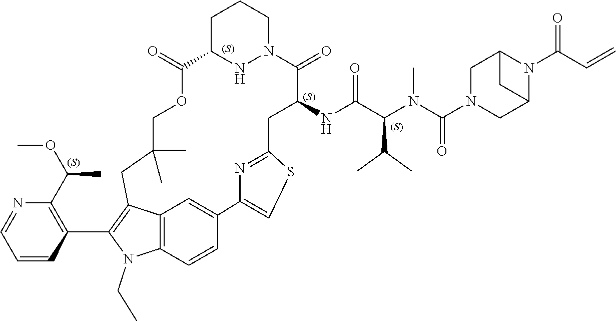 |
| BA 409 | 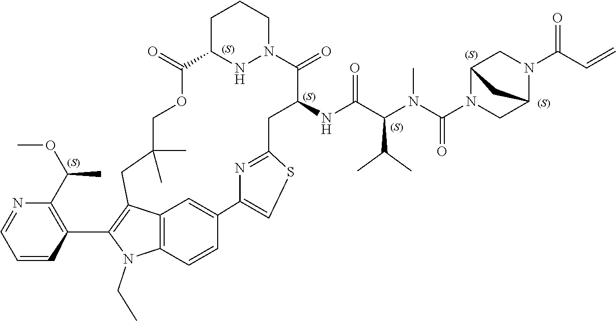 |
| BA 410 | 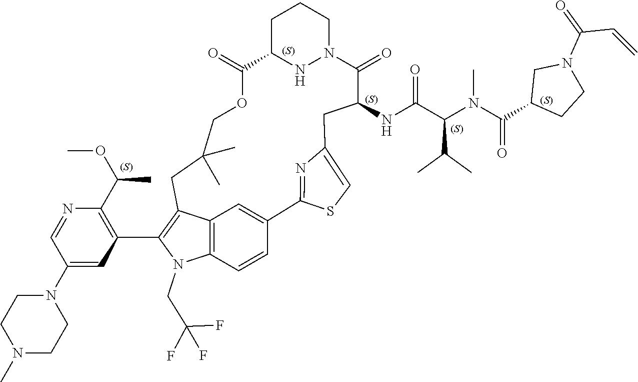 |
| BA 411 | 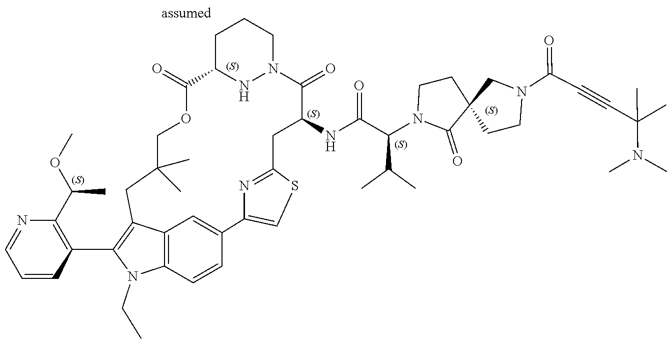 |
| BA 412 | 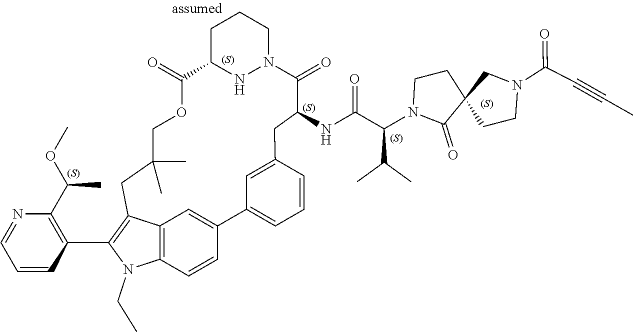 |
| BA 413 | 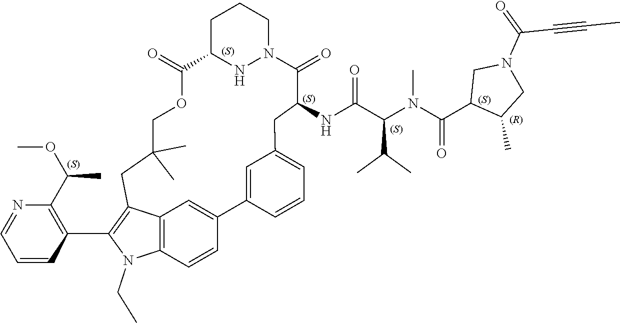 |
| BA 414 | 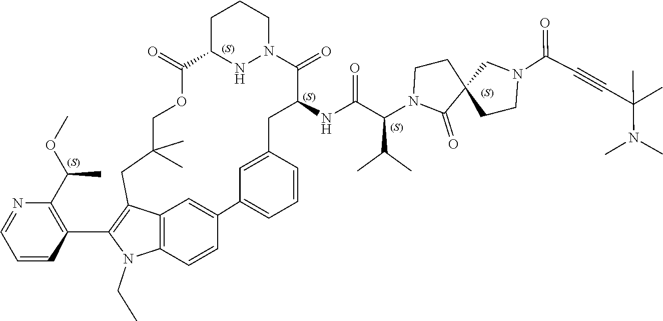 |
| BA 415 | 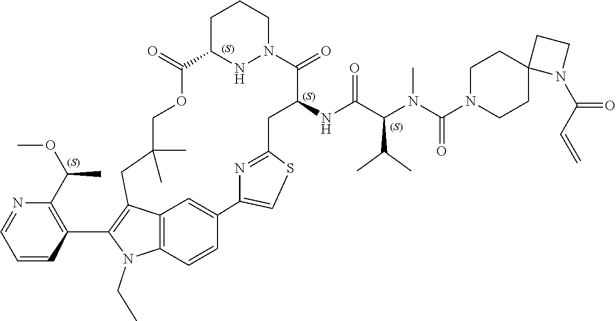 |
| BA 416 | 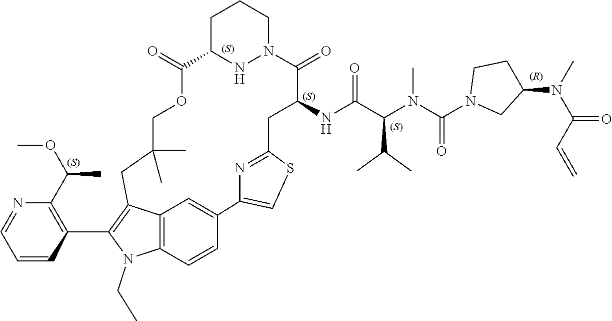 |
| BA 417 | 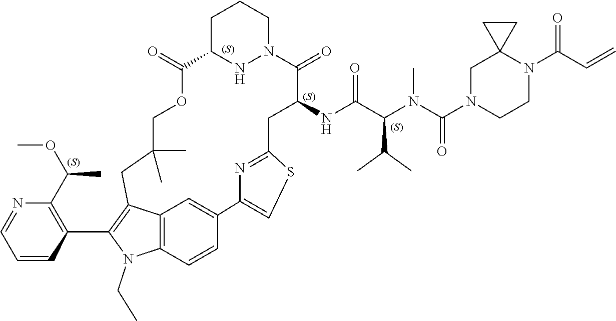 |
| BA 418 | 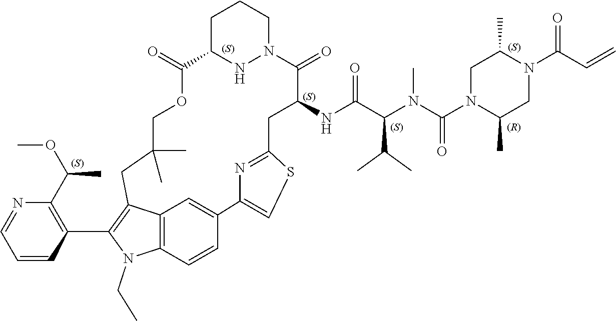 |
| BA 419 | 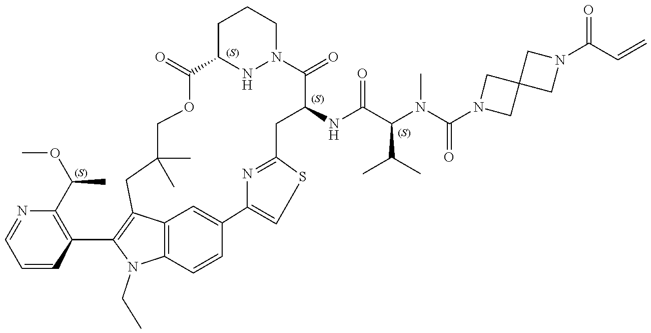 |
| BA 420 | 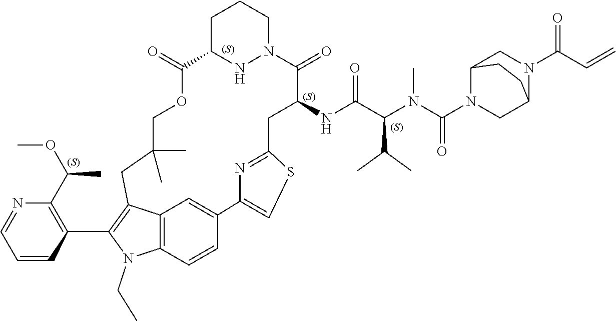 |
| BA 421 | 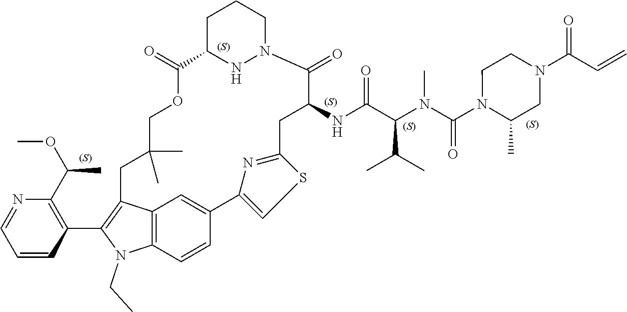 |
| BA 422 | 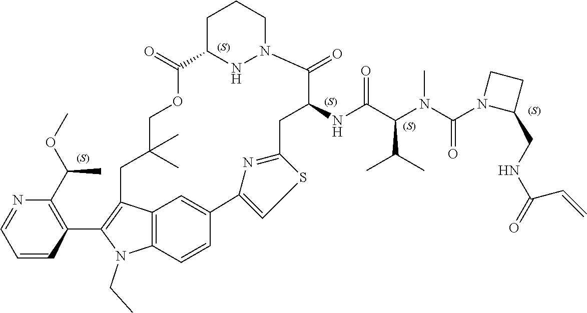 |
| BA 423 | 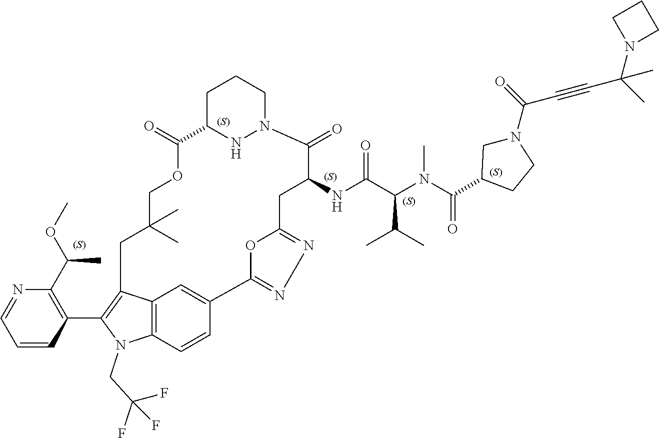 |
| BA 424 | 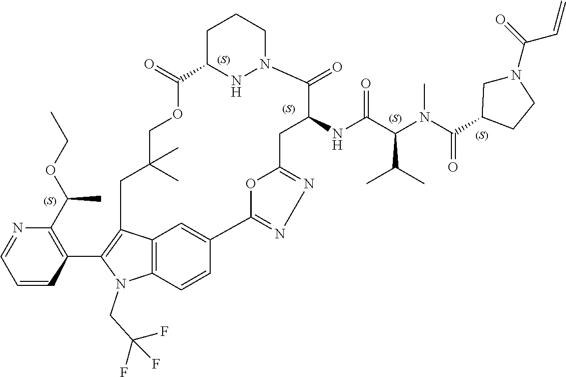 |
| BA 425 | 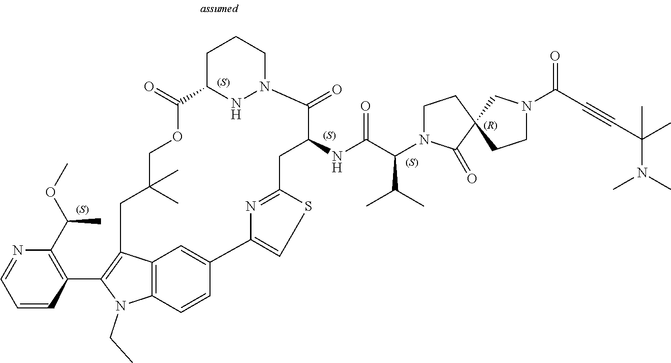 |
| BA 426 | 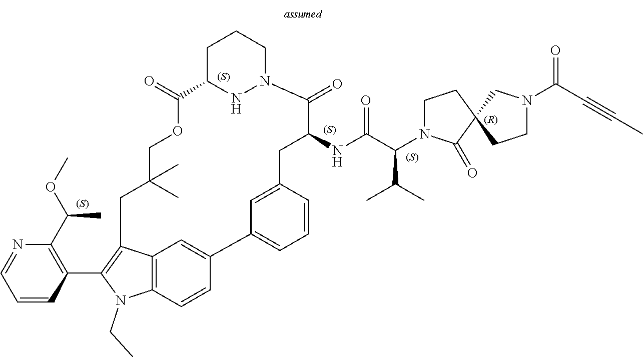 |
| BA 427 | 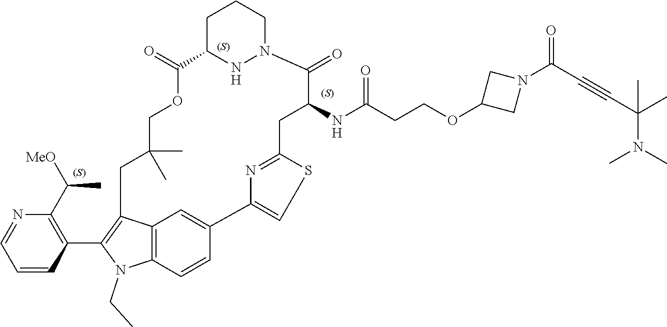 |
| BA 428 | 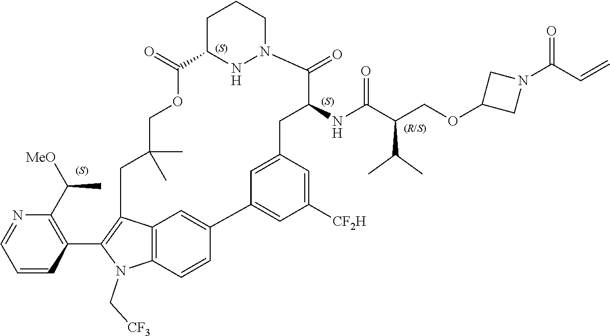 |
| BA 429 | 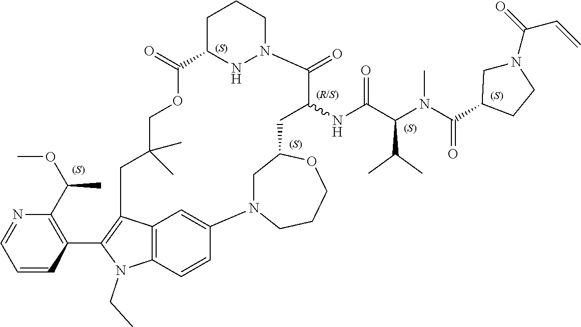 |
| BA 430 | 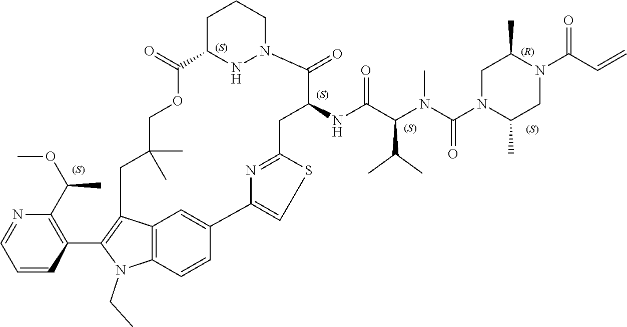 |
| BA 431 | 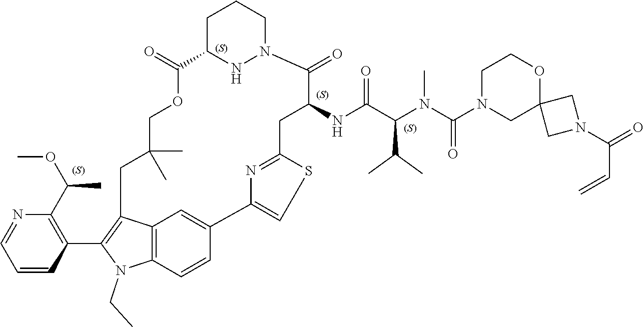 |
| BA 432 | 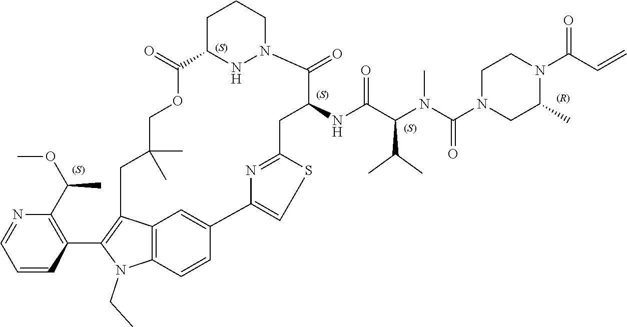 |
| BA 433 | 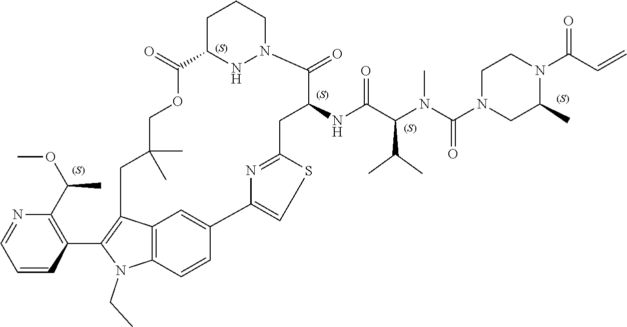 |
| BA 334 | 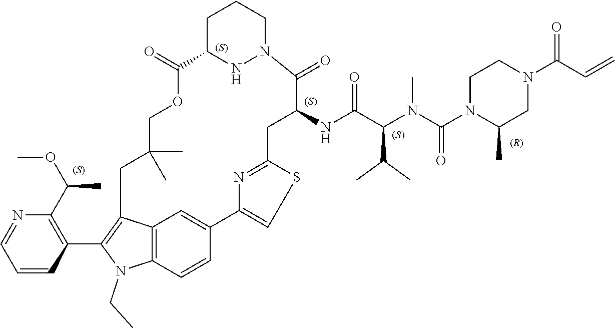 |
| BA 435 | 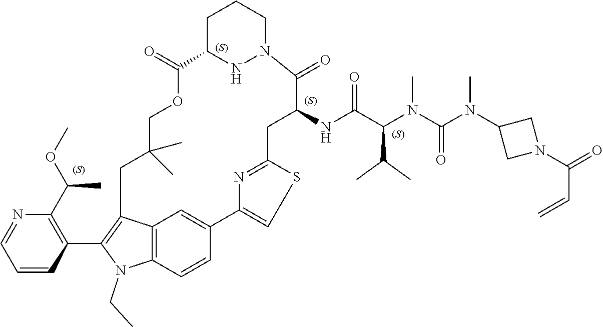 |
| BA 436 | 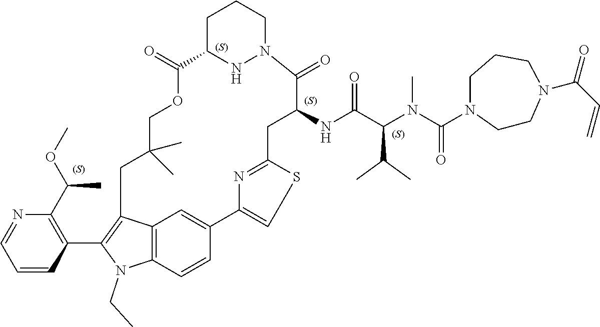 |
| BA 437 | 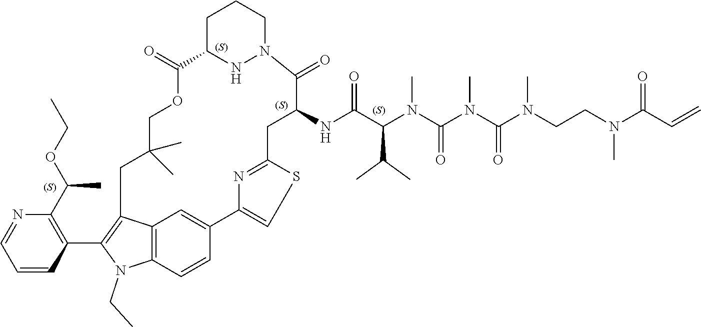 |
| BA 438 | 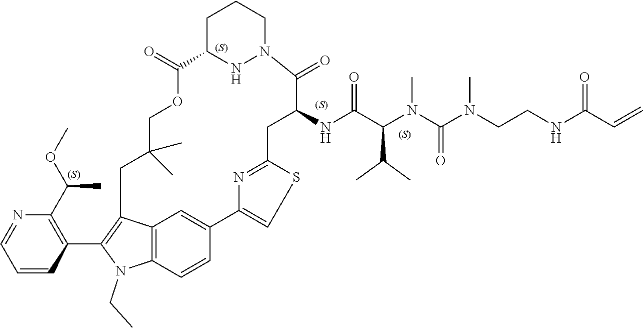 |
| BA 439 | 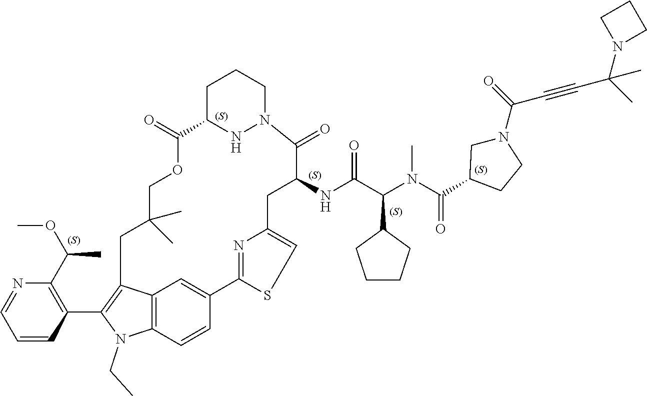 |
| BA 440 | 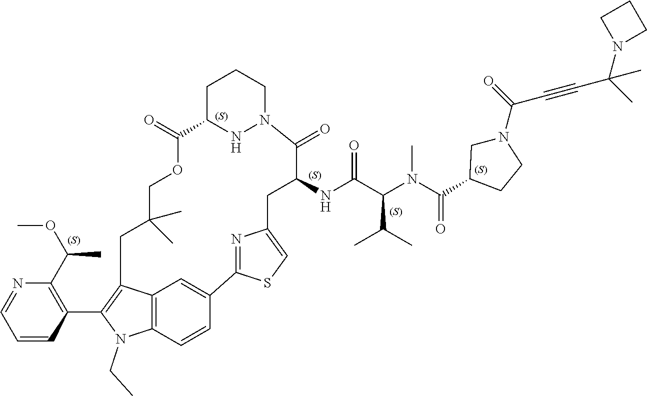 |
| BA 441 | 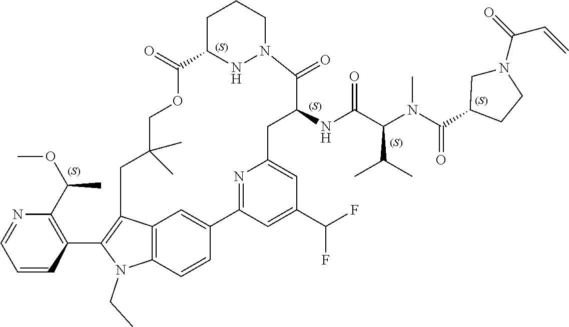 |
| BA 442 | 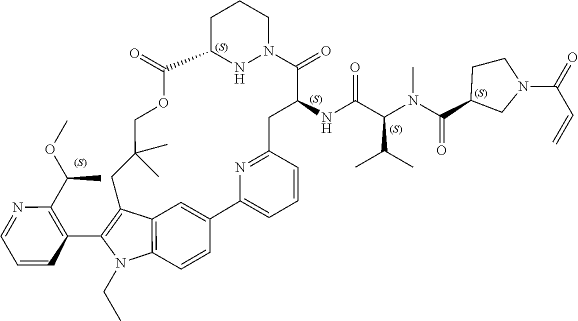 |
| BA 443 | 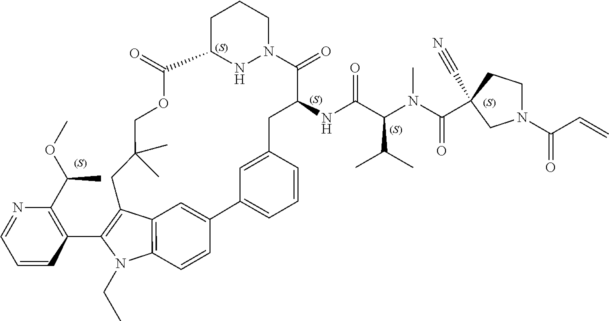 |
| BA 444 | 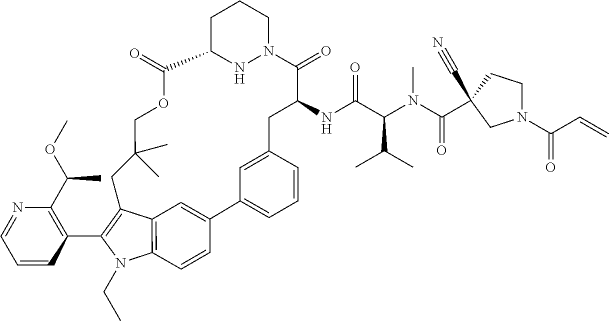 |
| BA 445 | 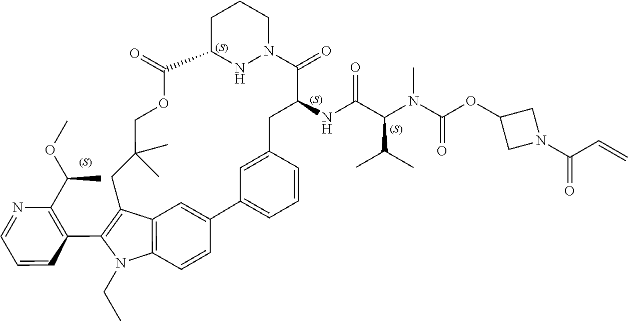 |
| BA 446 | 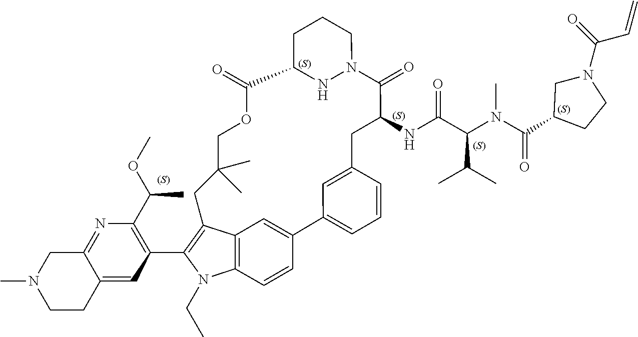 |
| BA 447 | 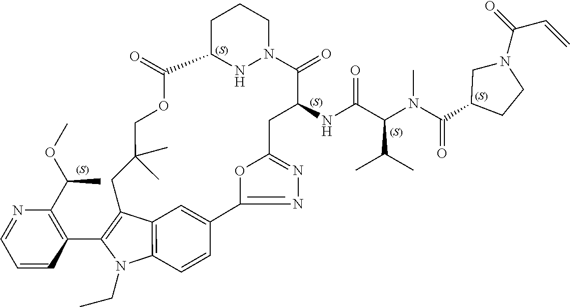 |
| BA 448 | 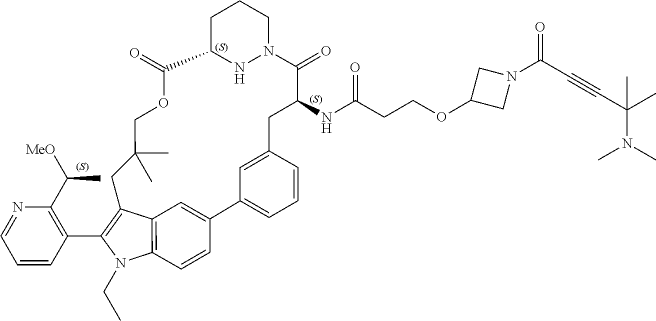 |
| BA 449 | 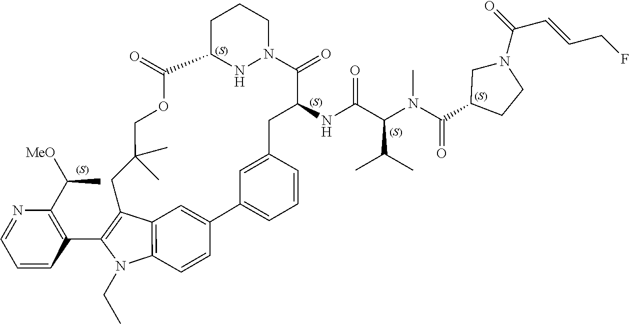 |
| BA 450 | 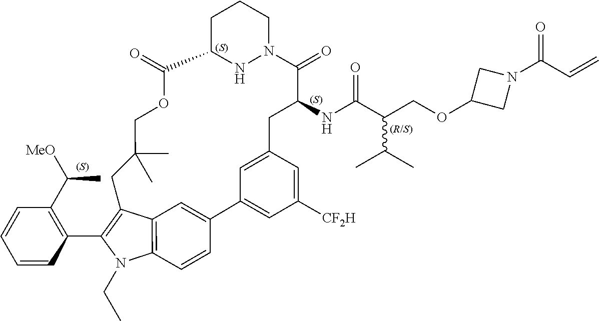 |
| BA 451 | 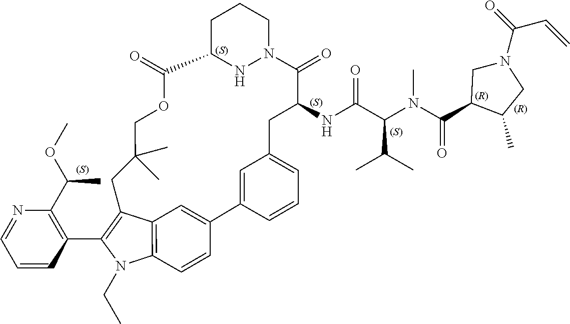 |
| BA 452 | 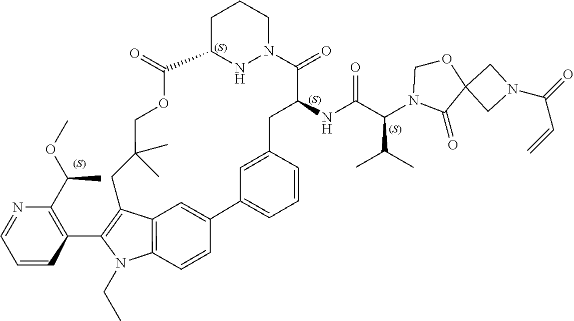 |
| BA 453 | 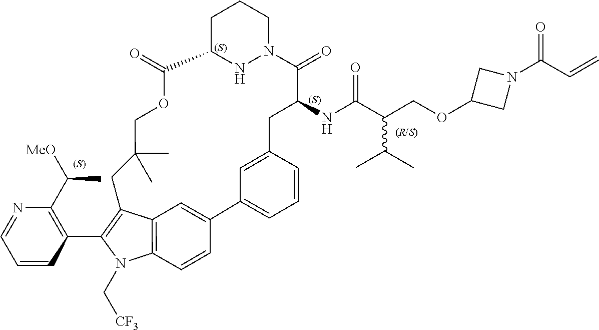 |
| BA 454 | 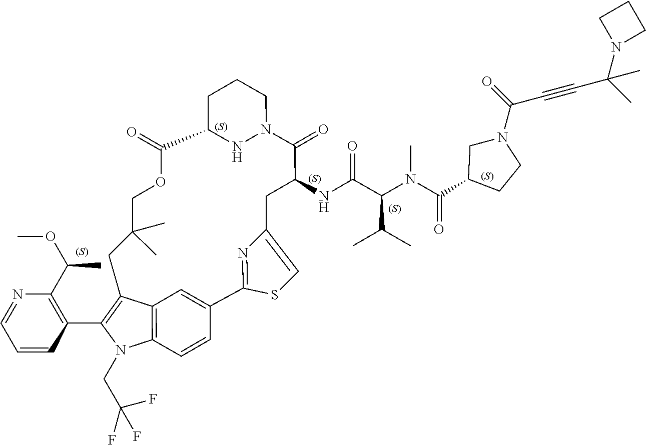 |
| BA 455 | 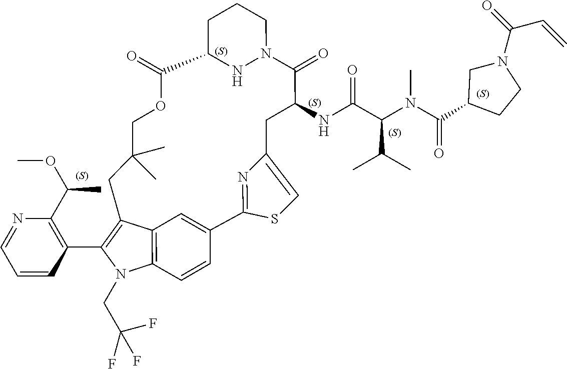 |
| BA 456 | 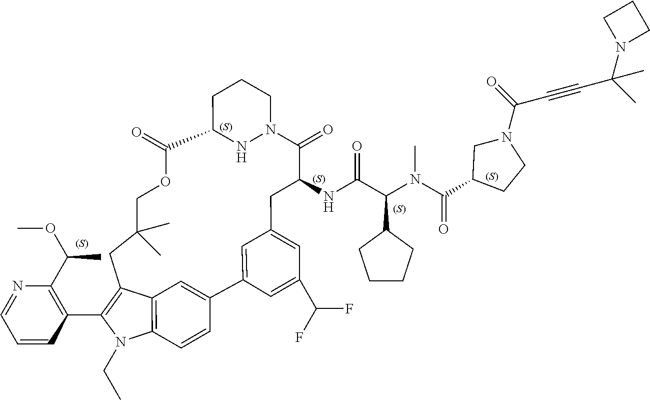 |
| BA 457 | 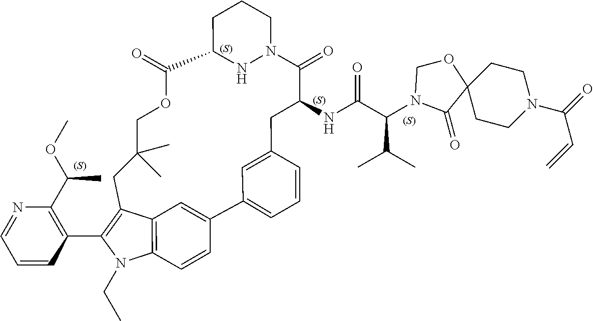 |
| BA 458 | 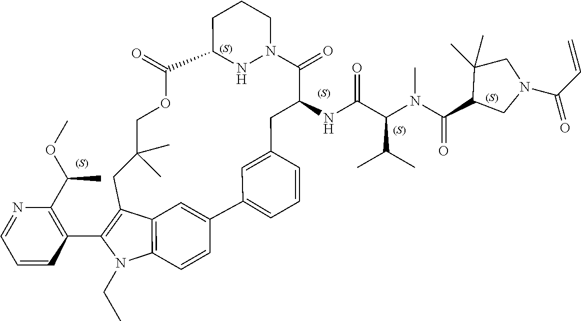 |
| BA 459 | 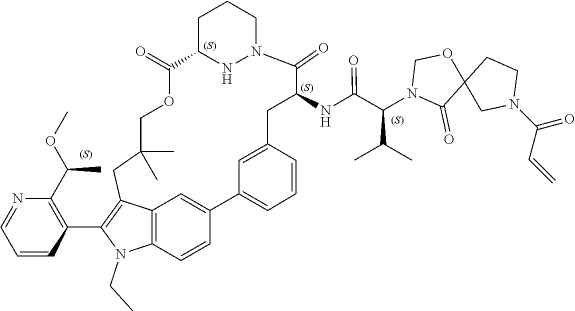 |
| BA 460 | 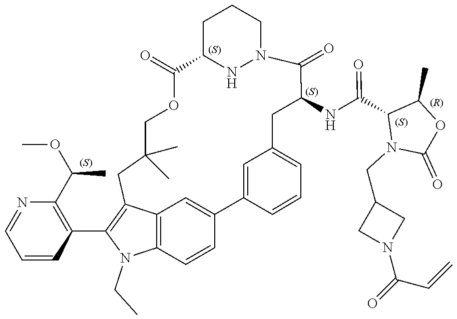 |
| BA 461 | 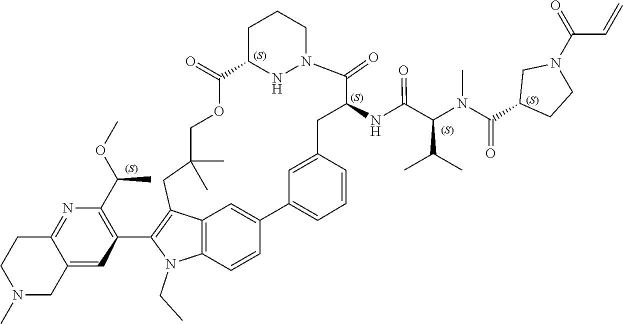 |
| BA 462 | 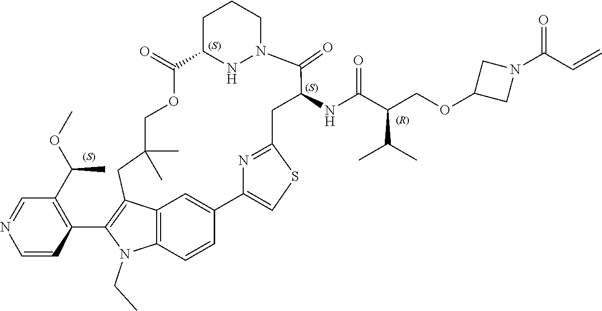 |
| BA 463 | 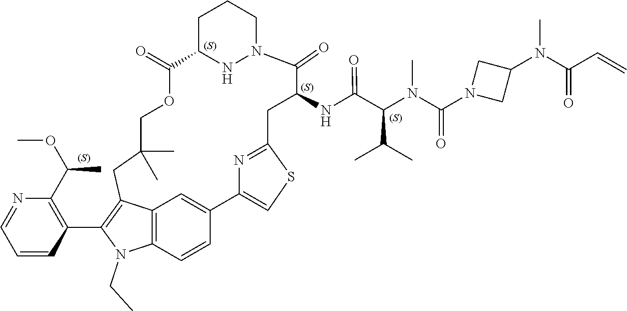 |
| BA 464 | 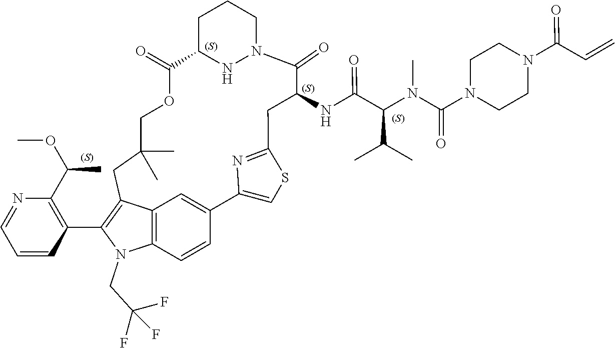 |
| BA 465 | 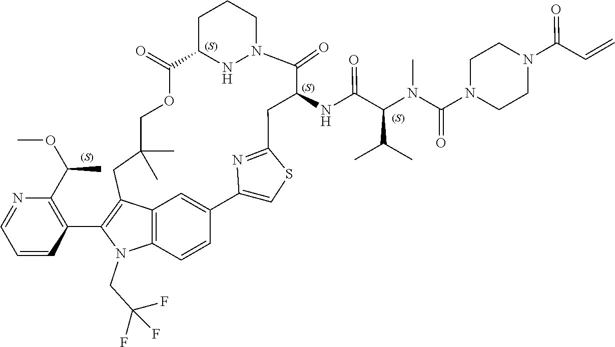 |
| BA 466 | 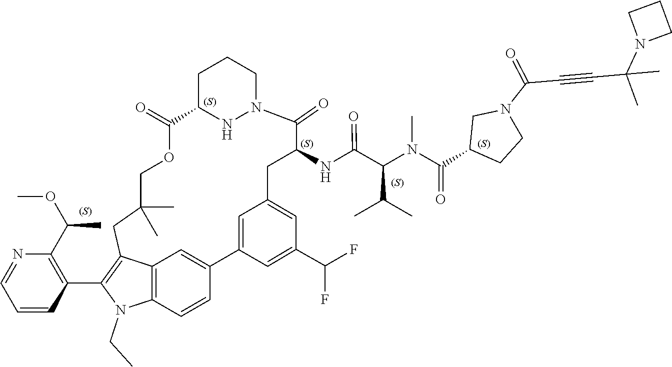 |
| BA 467 | 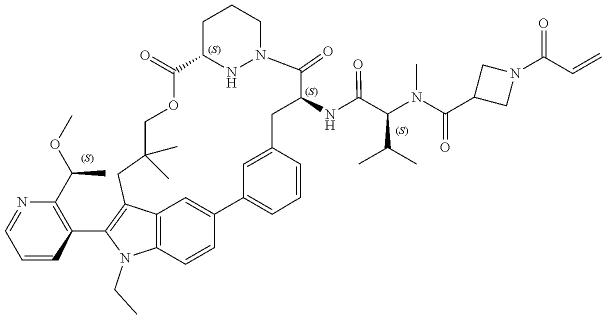 |
| BA 468 | 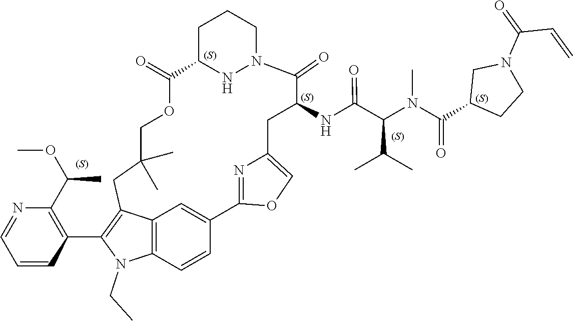 |
| BA 469 | 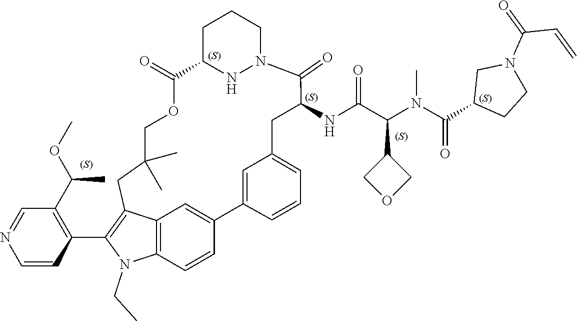 |
| BA 470 | 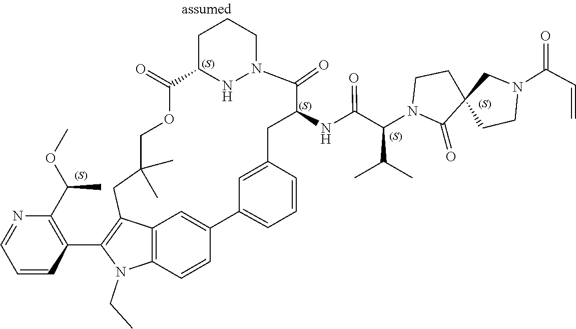 |
| BA 471 | 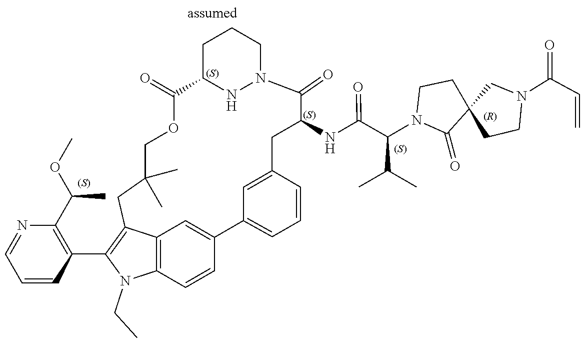 |
| BA 472 | 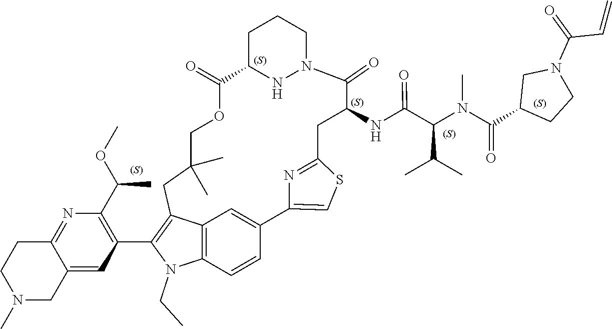 |
| BA 473 | 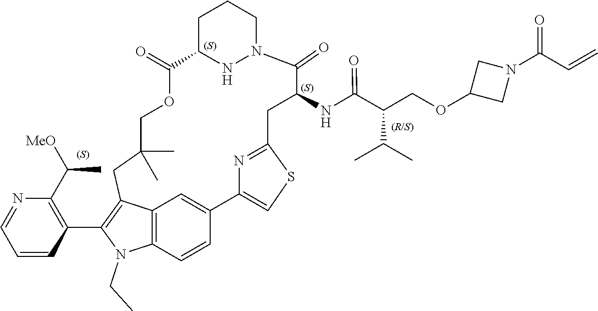 |
| BA 474 | 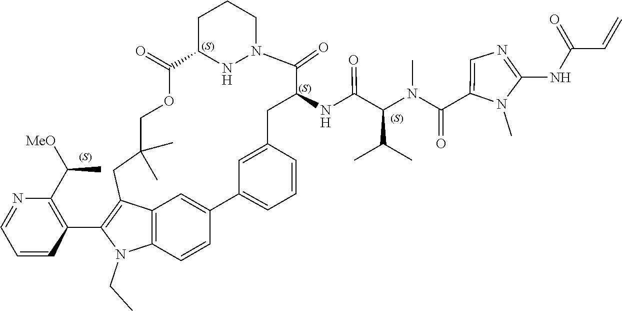 |
| BA 475 | 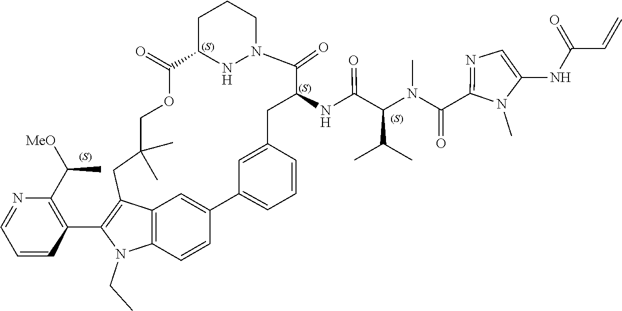 |
| BA 476 | 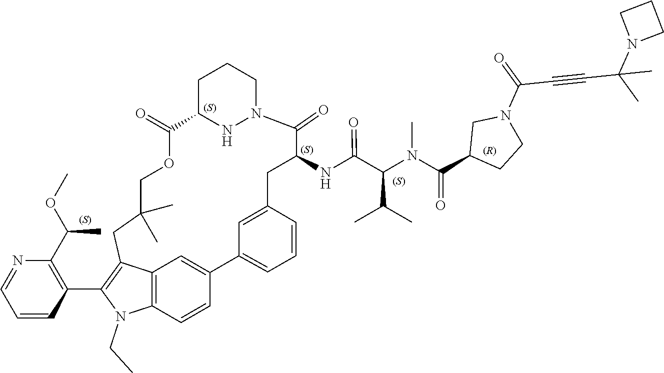 |
| BA 477 | 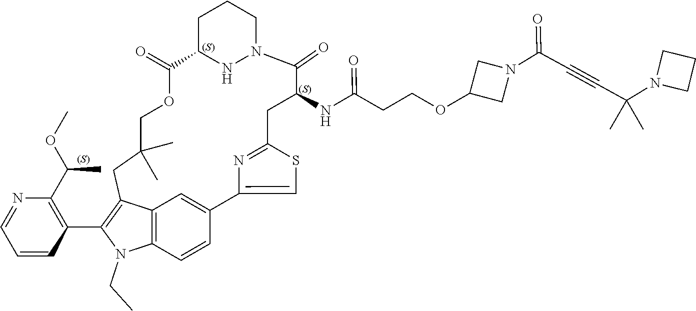 |
| BA 478 | 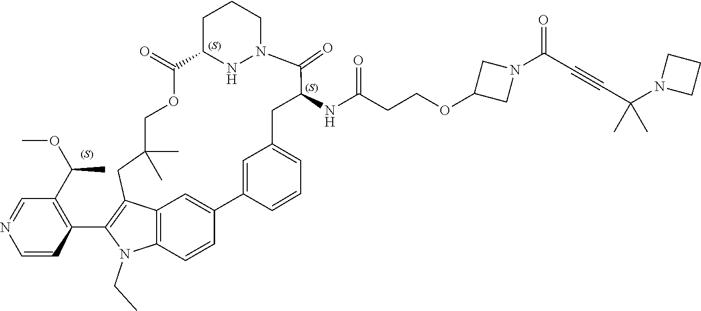 |
| BA 479 | 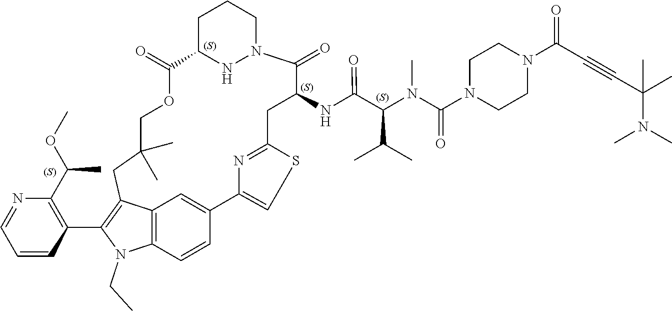 |
| BA 480 | 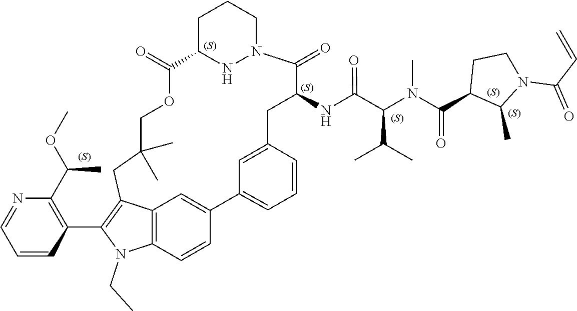 |
| BA 481 | 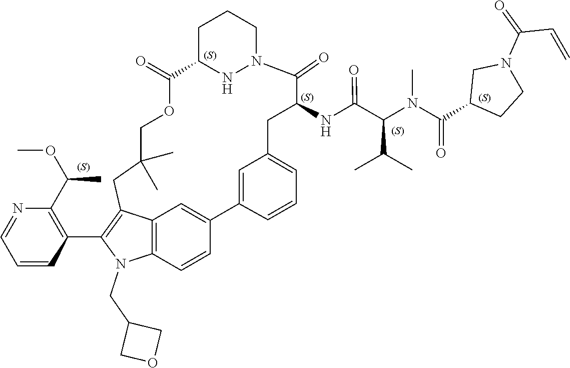 |
| BA 482 | 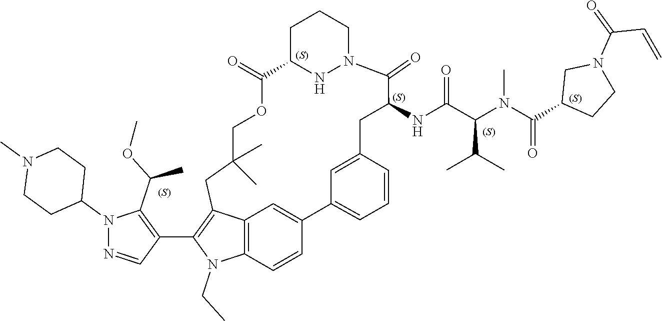 |
| BA 483 | 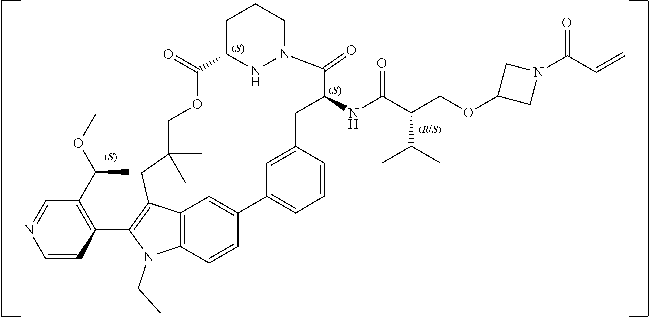 |
| BA 484 | 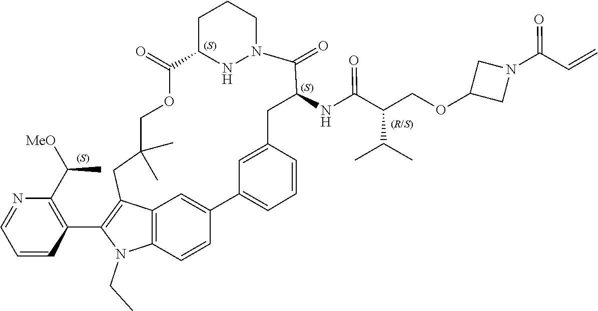 |
| BA 485 | 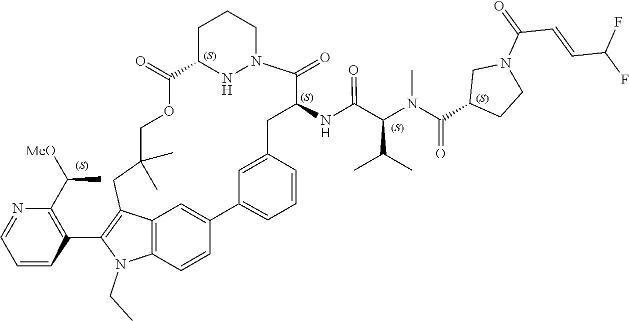 |
| BA 486 | 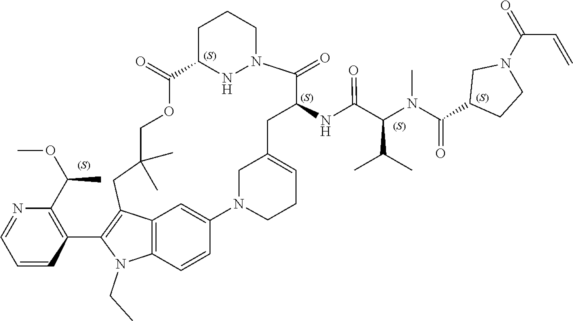 |
| BA 487 | 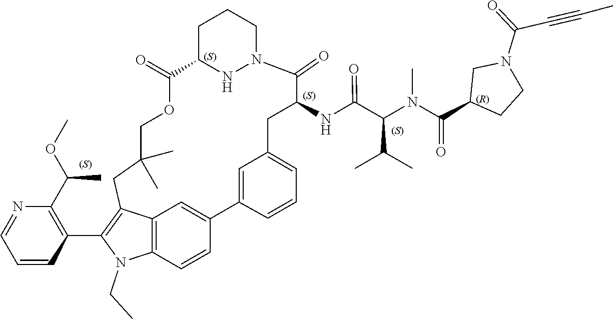 |
| BA 488 | 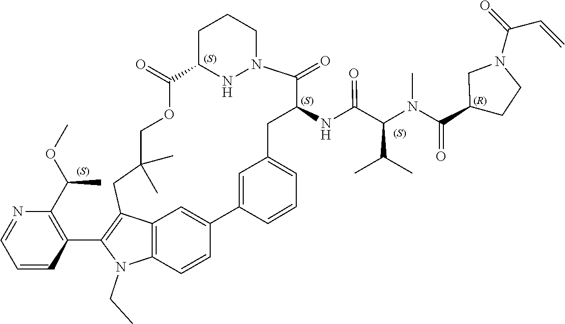 |
| BA 489 | 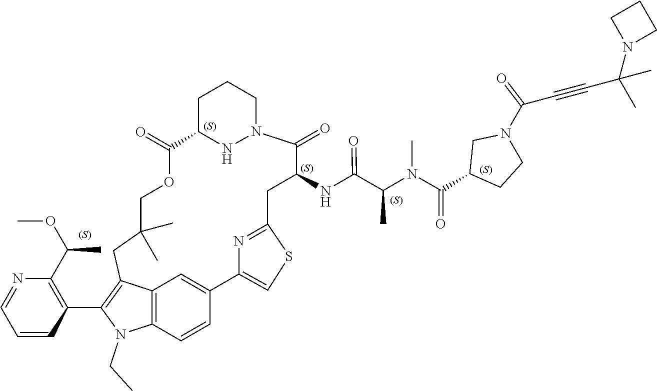 |
| BA 490 | 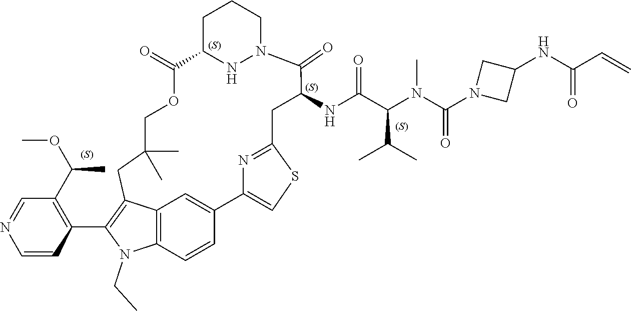 |
| BA 491 | 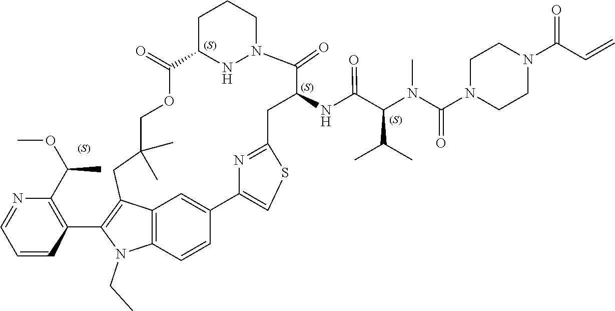 |
| BA 492 | 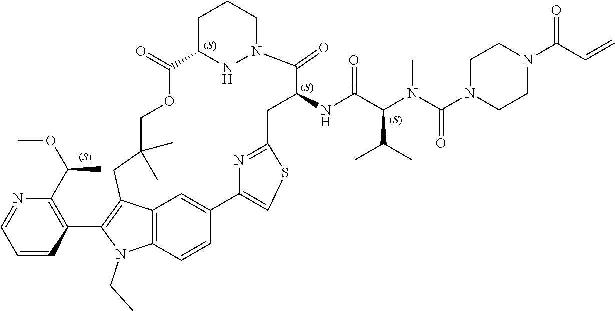 |
| BA 493 | 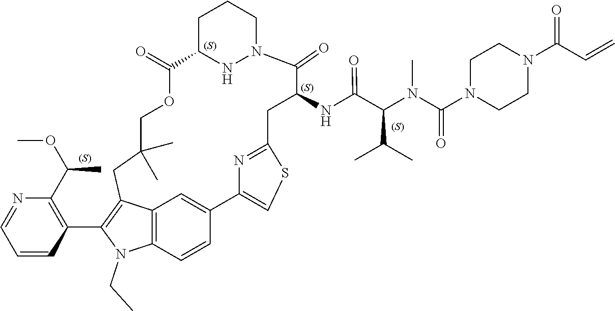 |
| BA 494 | 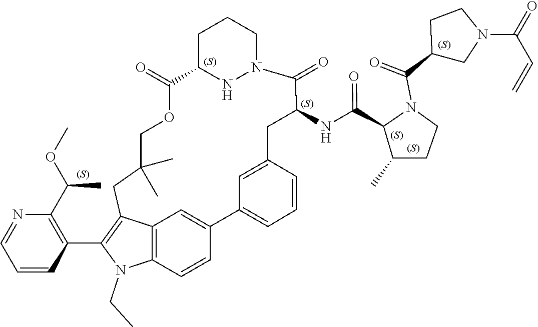 |
| BA 495 | 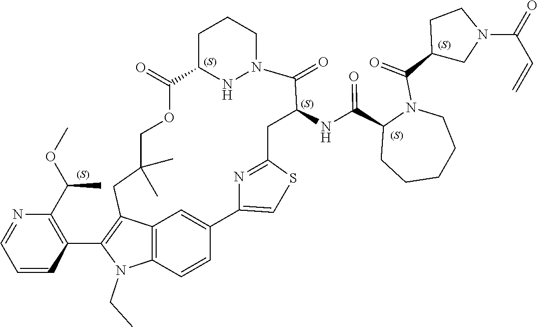 |
| BA 496 | 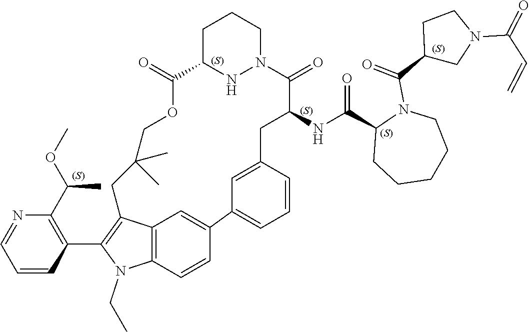 |
| BA 497 | 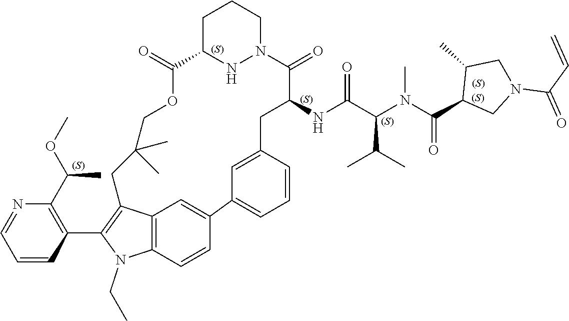 |
| BA 498 | 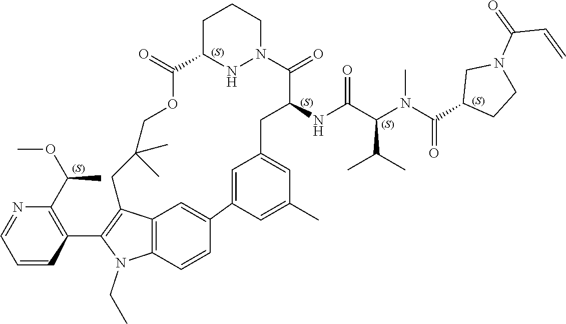 |
| BA 499 | 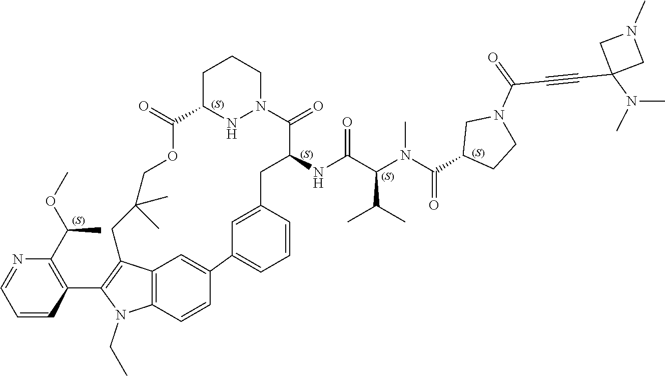 |
| BA 500 | 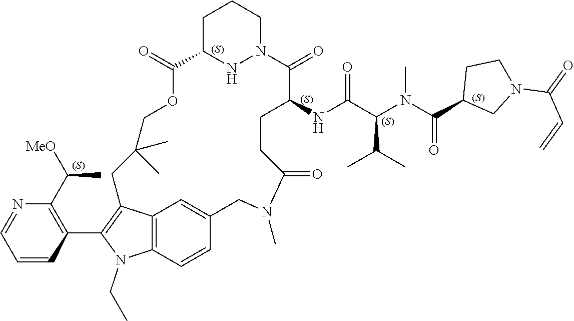 |
| BA 501 | 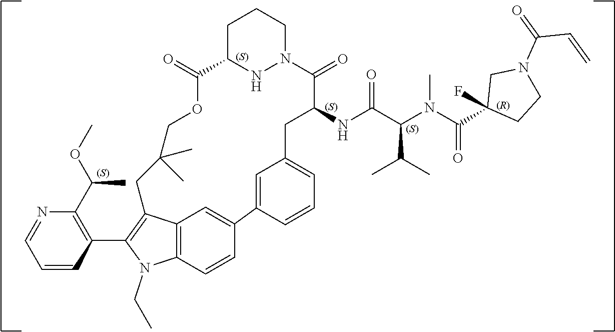 |
| BA 502 | 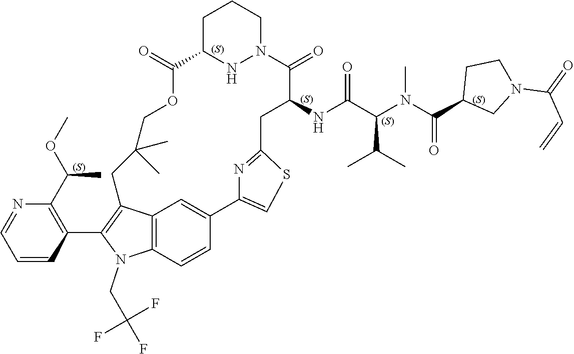 |
| BA 503 | 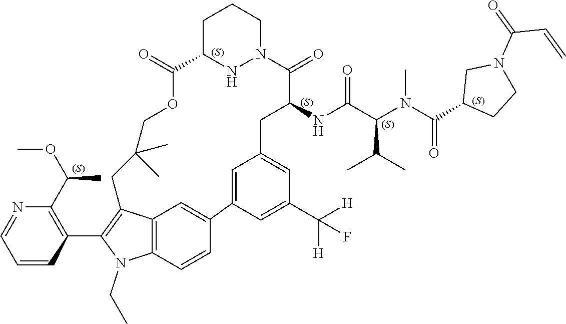 |
| BA 504 | 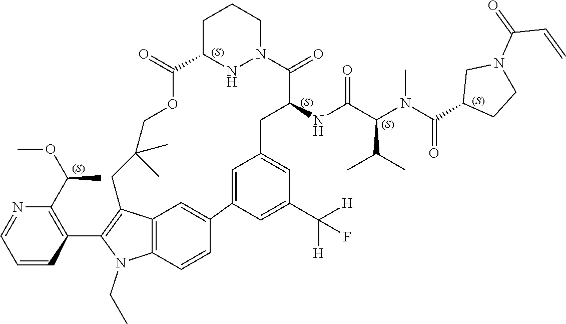 |
| BA 505 | 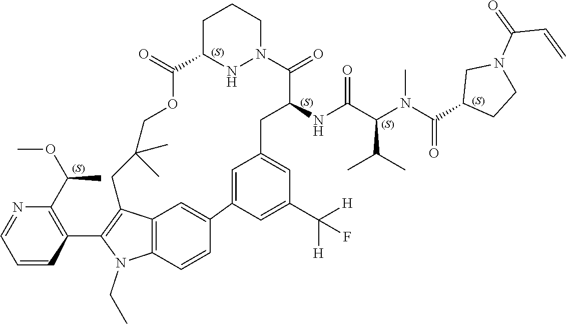 |
| BA 506 | 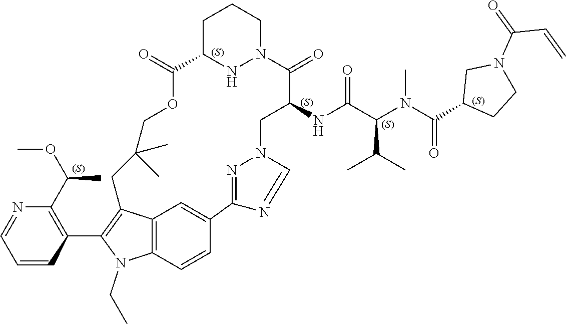 |
| BA 507 | 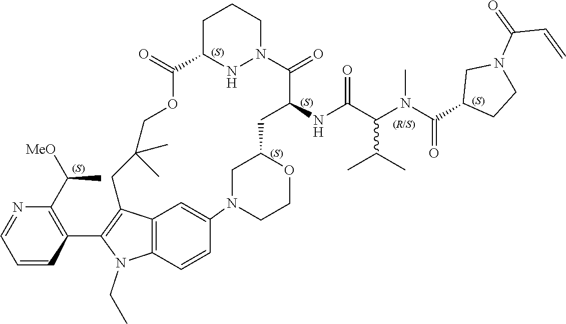 |
| BA 508 | 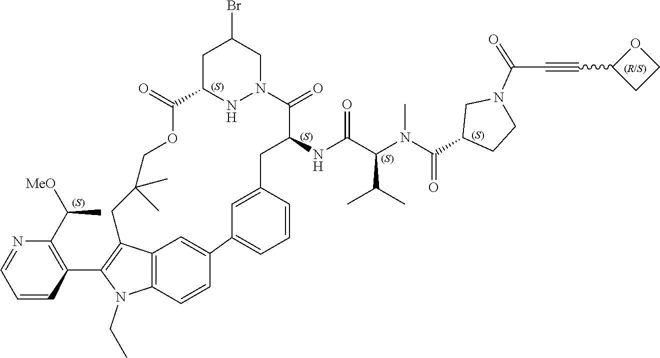 |
| BA 509 | 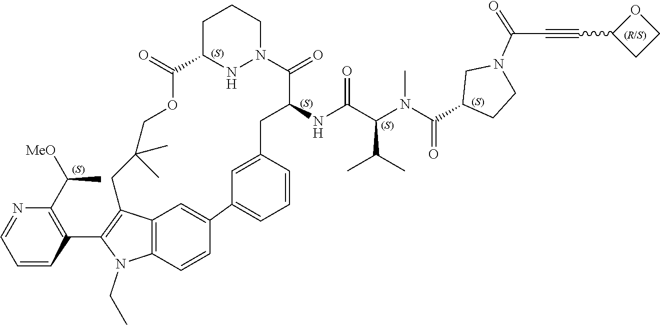 |
| BA 510 | 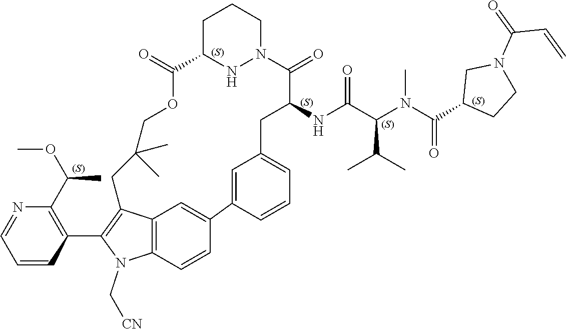 |
| BA 511 | 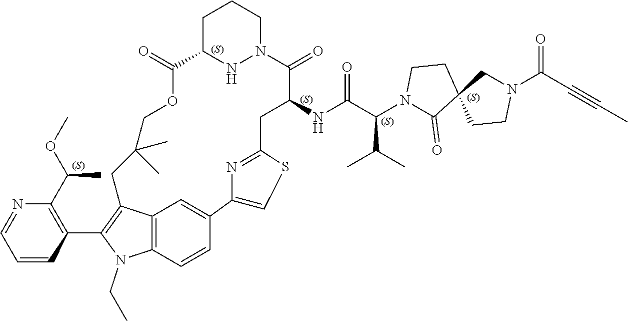 |
| BA 512 | 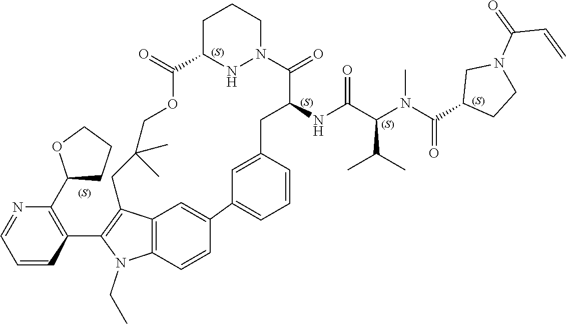 |
| BA 513 | 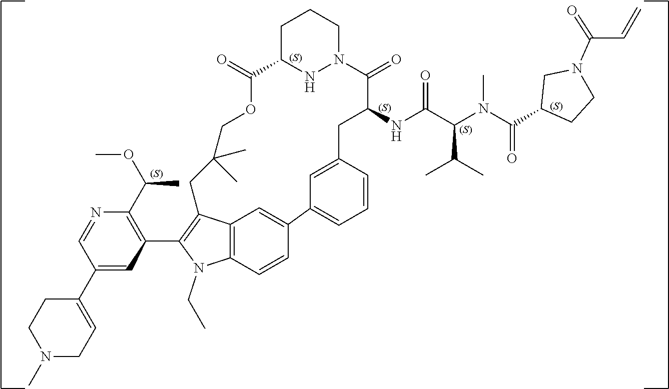 |
| BA 514 | 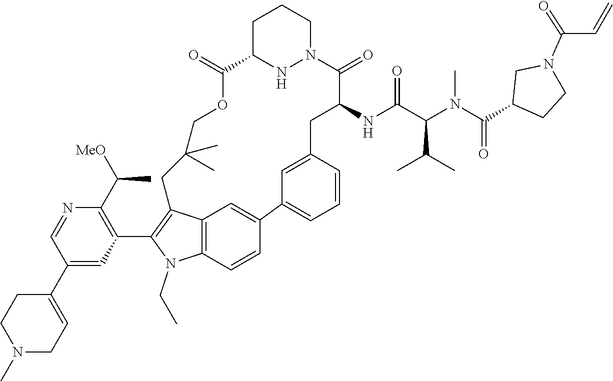 |
| BA 515 | 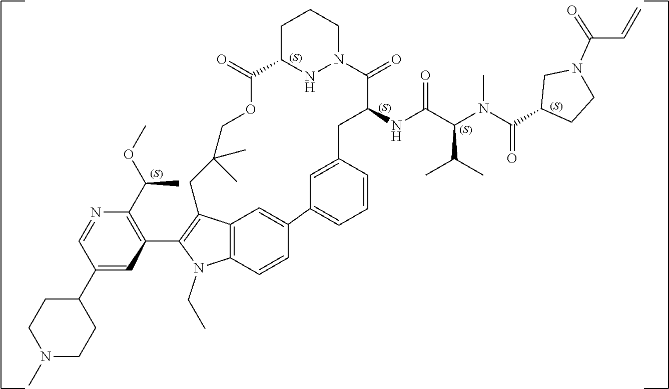 |
| BA 516 | 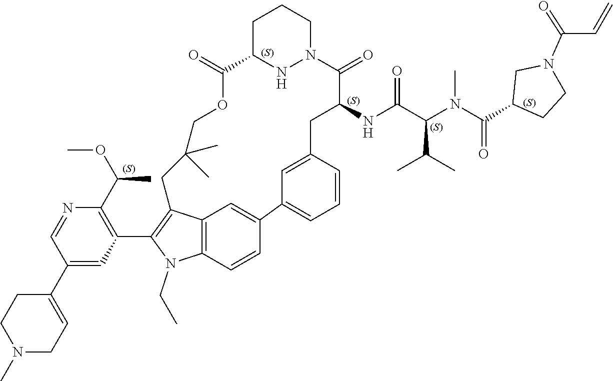 |
| BA 517 | 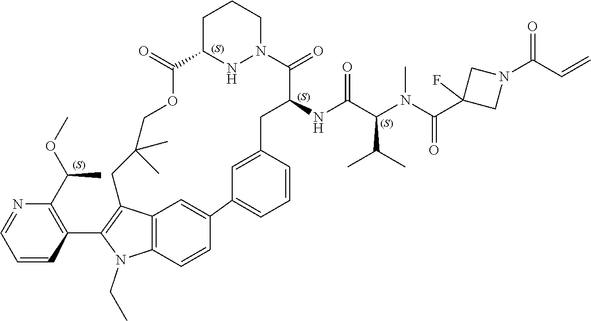 |
| BA 518 | 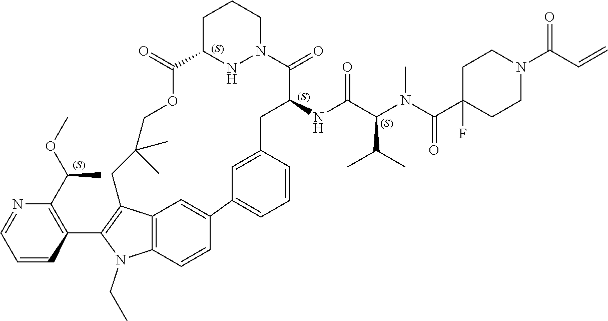 |
| BA 519 | 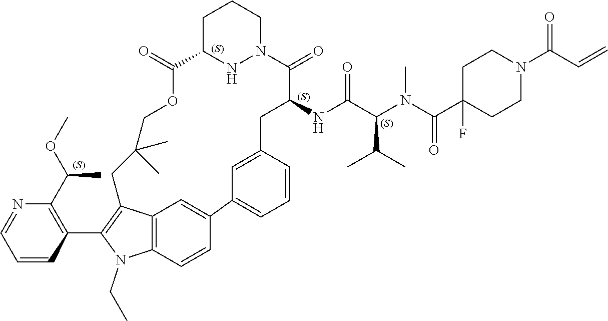 |
| BA 520 | 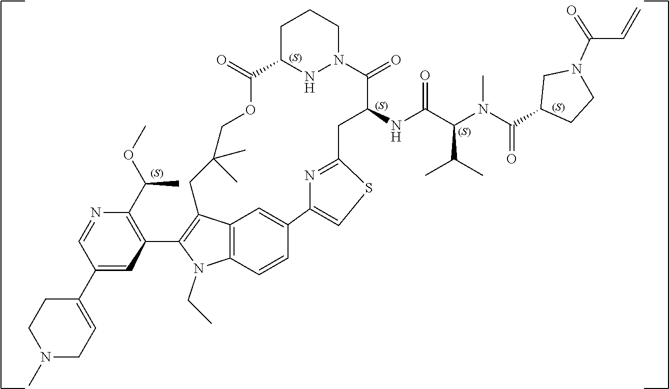 |
| BA 521 | 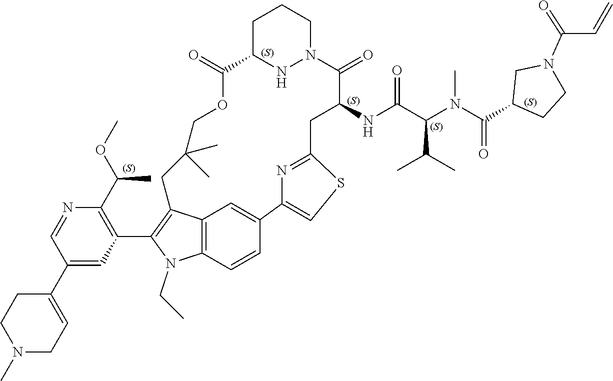 |
| BA 522 | 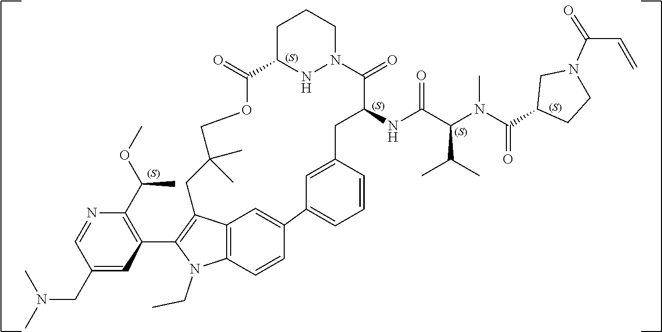 |
| BA 523 | 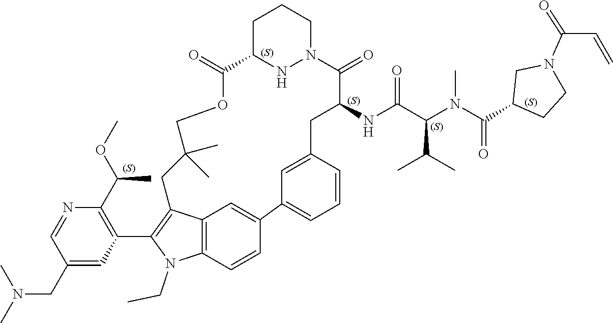 |
| BA 524 | 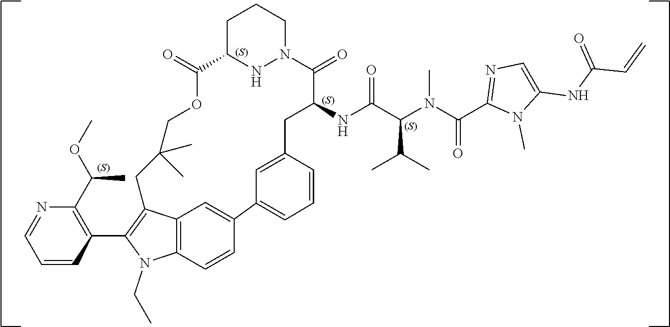 |
| BA 525 | 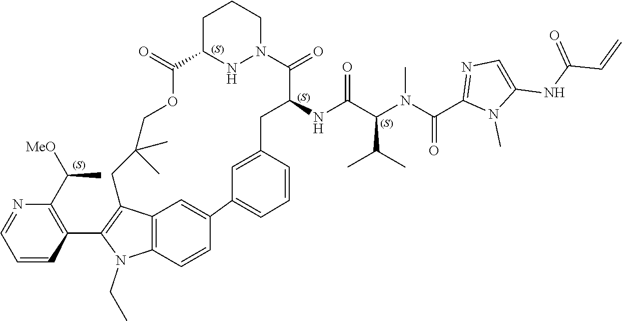 |
| BA 526 | 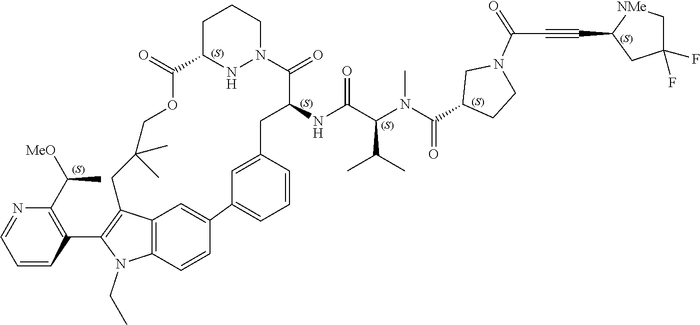 |
| BA 527 | 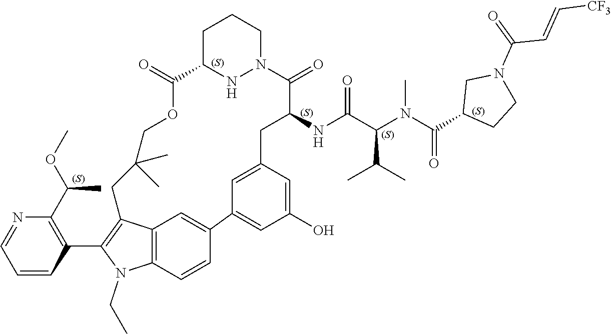 |
| BA 528 | 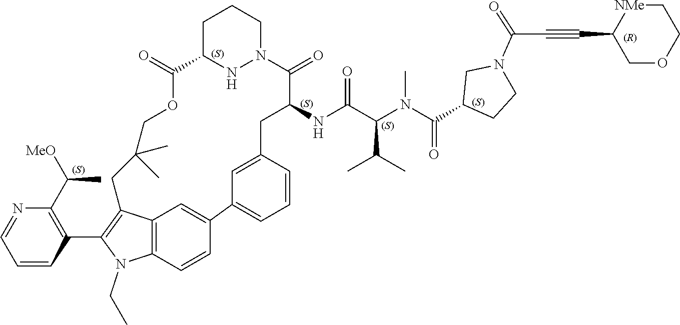 |
| BA 529 | 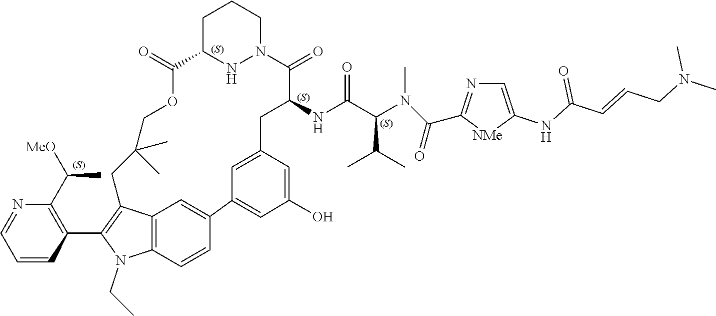 |
| BA 530 | 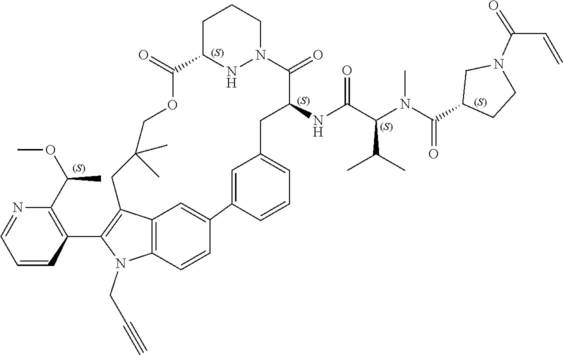 |
| BA 531 | 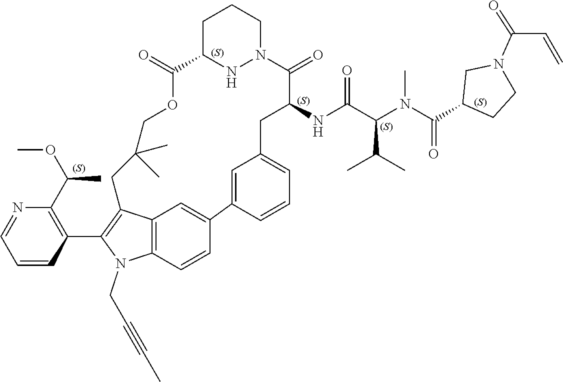 |
| BA 532 | 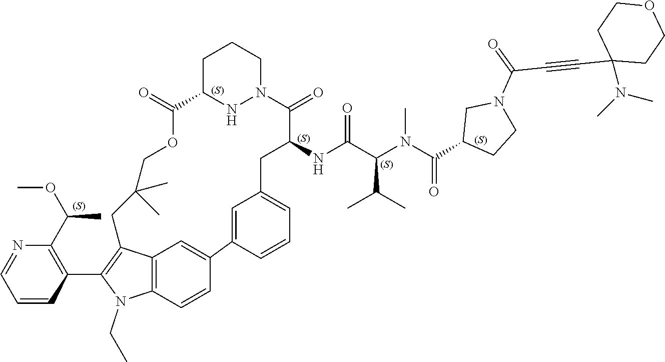 |
| BA 533 | 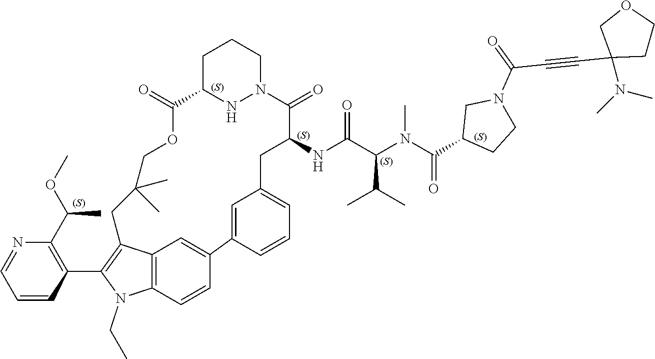 |
| BA 534 | 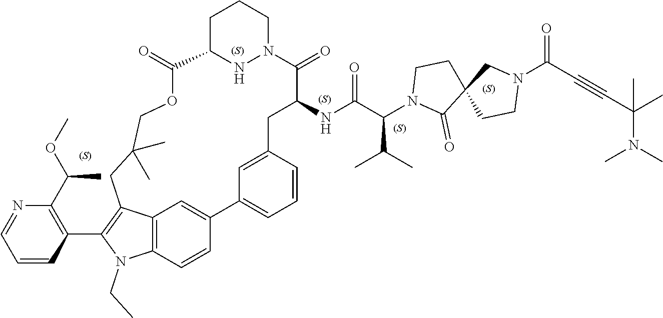 |
| BA 535 | 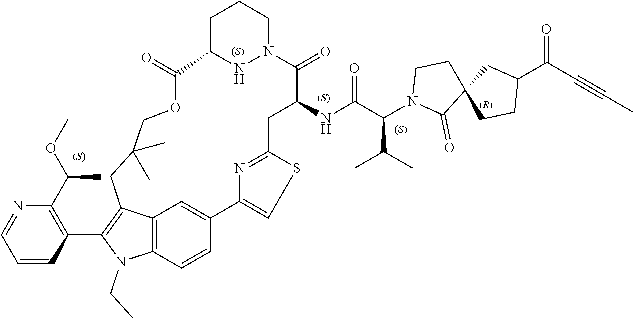 |
| BA 536 | 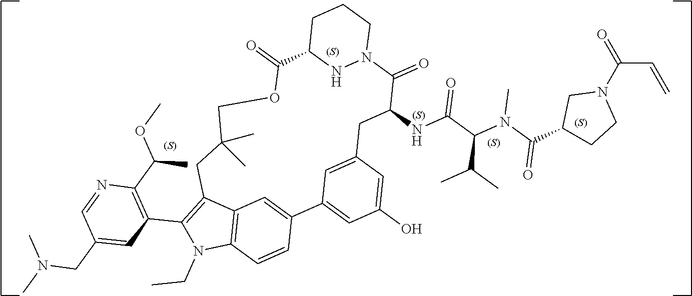 |
| BA 537 | 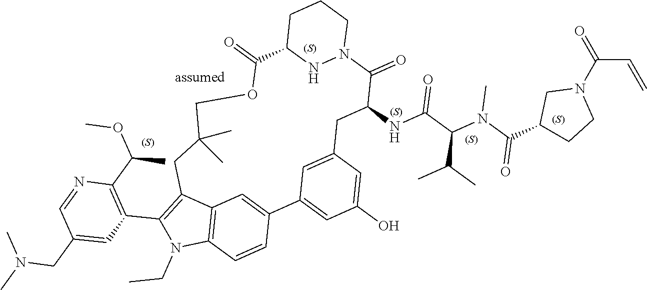 |
| BA 538 | 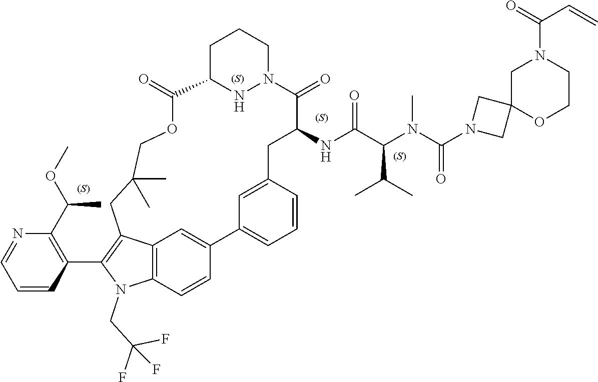 |
| BA 539 | 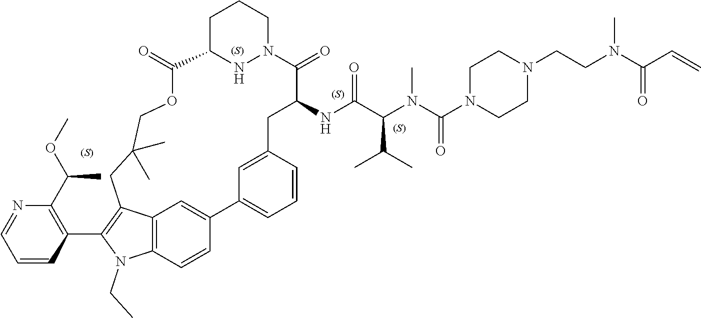 |
| BA 540 | 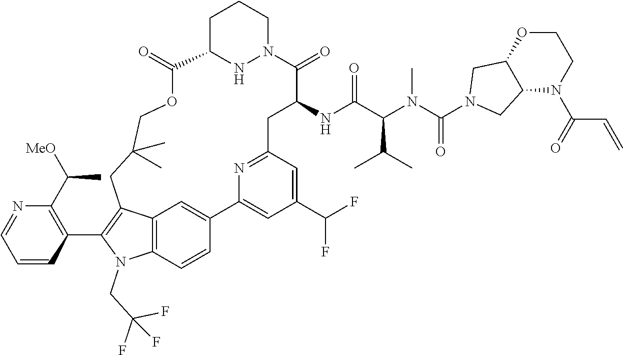 |
| BA 541 | 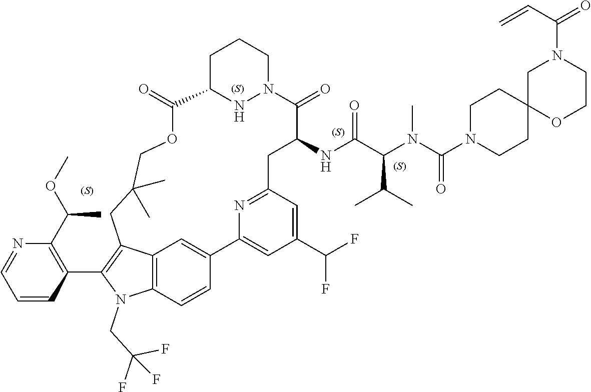 |
| BA 542 | 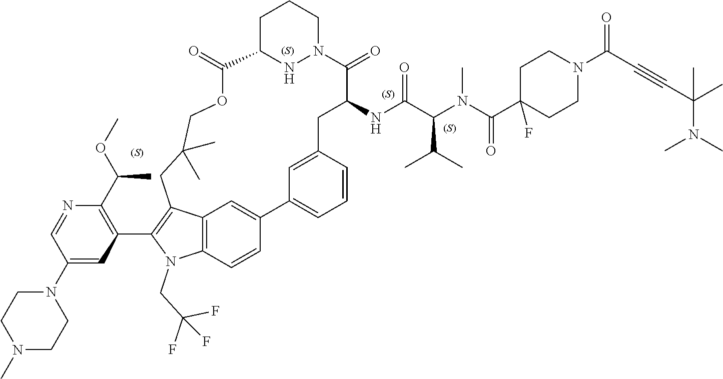 |
| BA 543 | 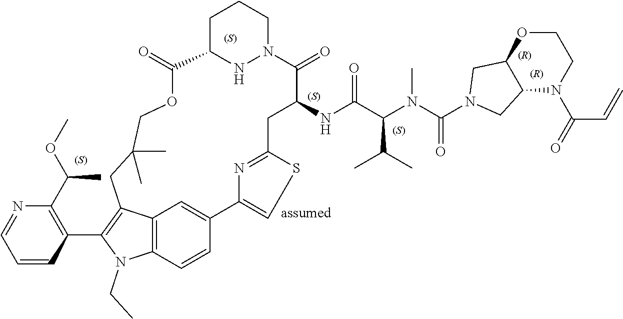 |
| BA 544 | 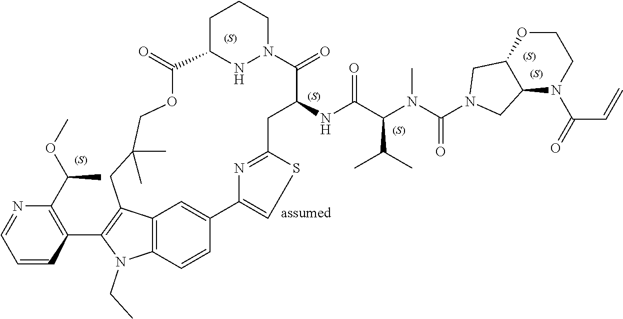 |
| BA 545 | 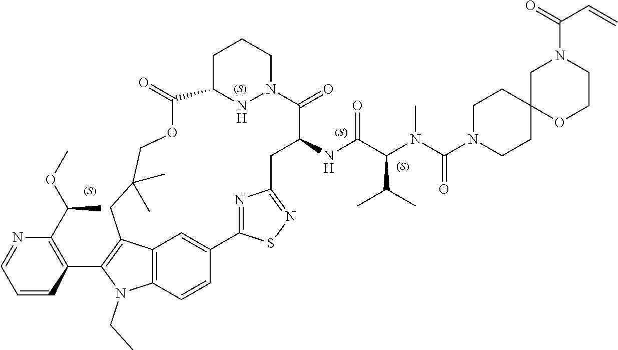 |
| BA 546 | 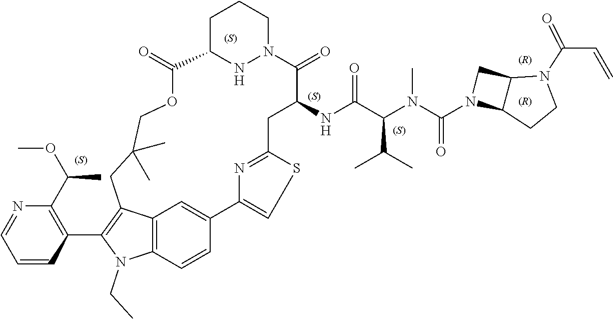 |
| BA 547 | 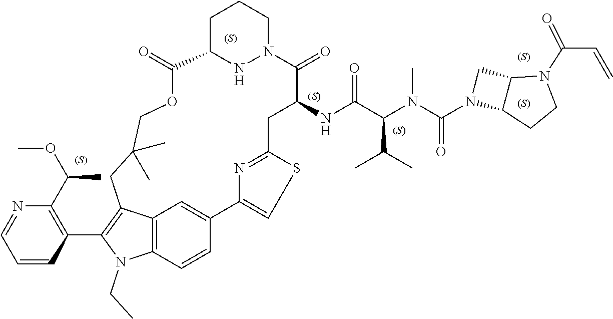 |
| BA 548 | 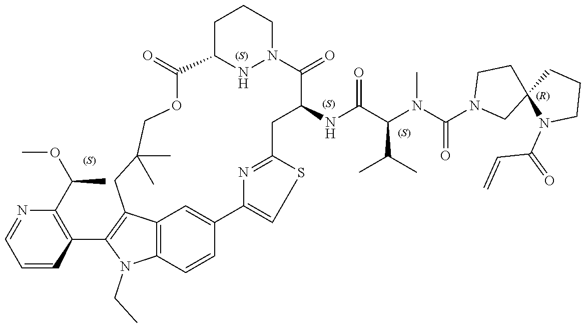 |
| BA 549 | 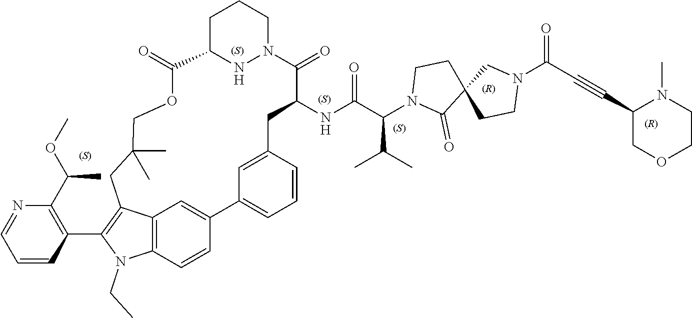 |
| BA 550 | 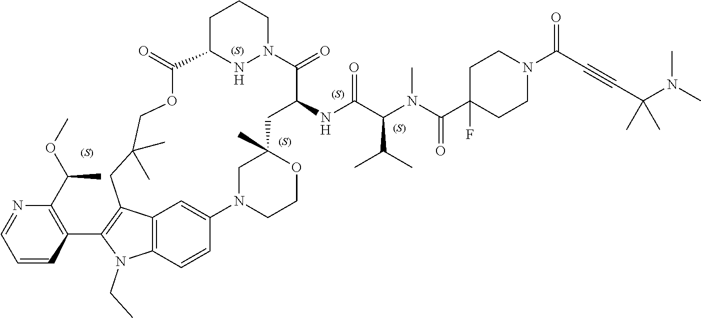 |
| BA 551 | 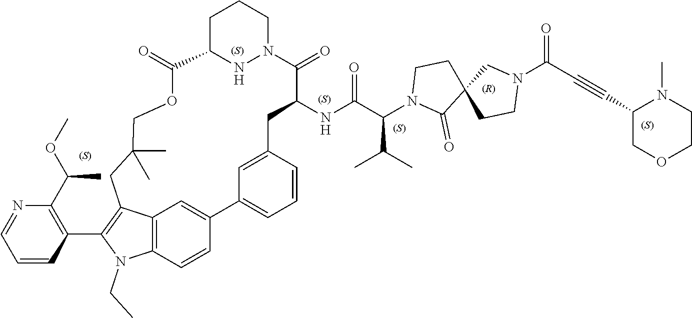 |
| BA 552 | 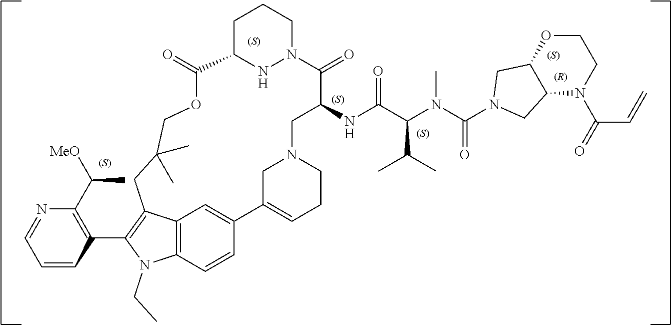 |
| BA 553 | 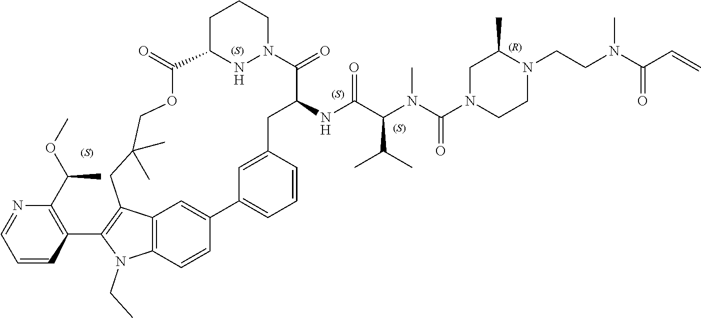 |
| BA 554 | 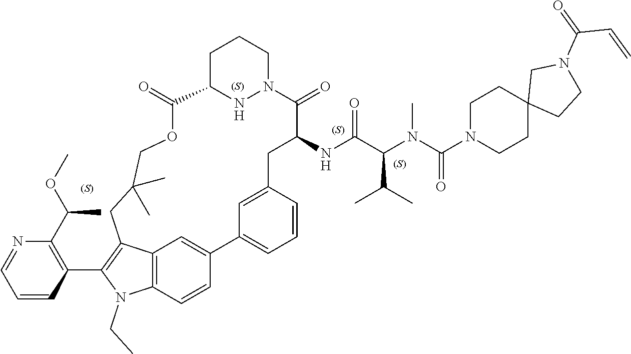 |
| BA 555 | 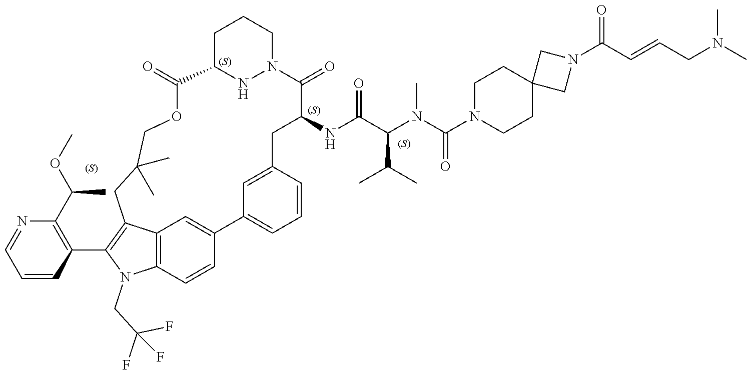 |
| BA 556 | 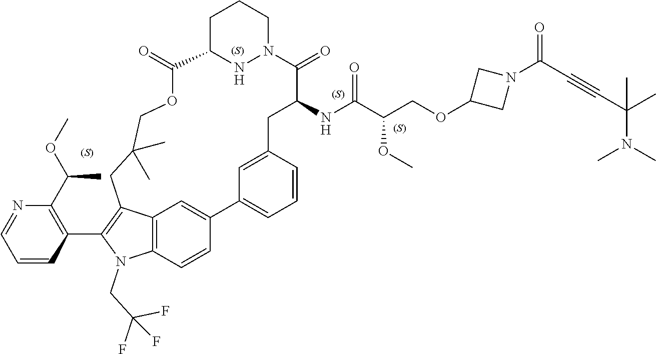 |
| BA 557 | 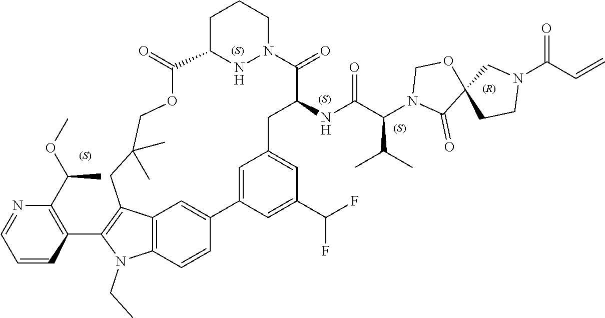 |
| BA 558 | 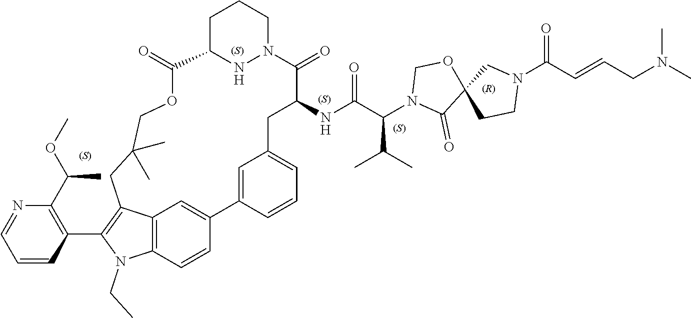 |
| BA 559 | 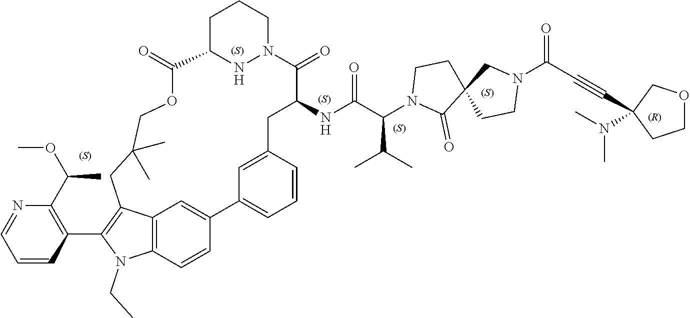 |
| BA 560 | 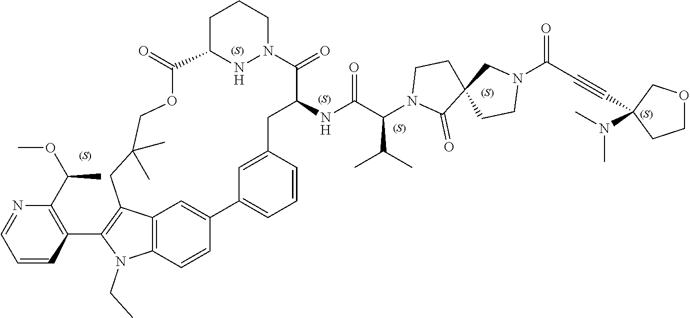 |
| BA 561 | 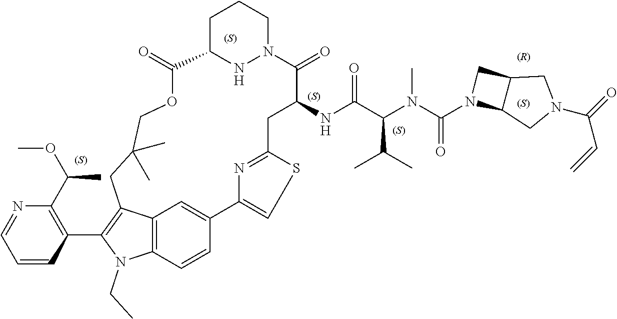 |
| BA 562 | 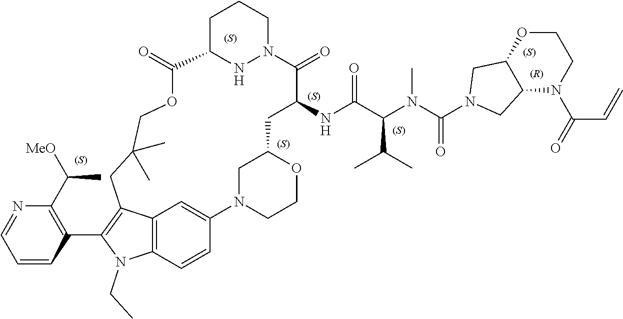 |
| BA 563 | 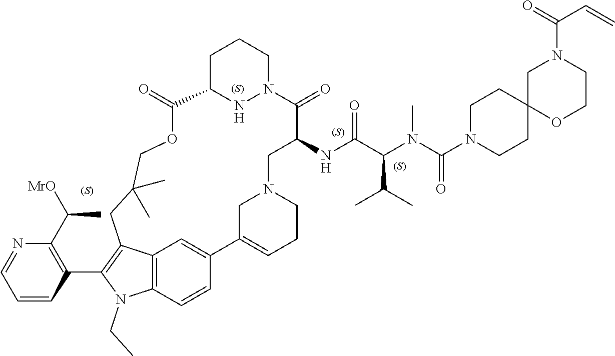 |
| BA 564 | 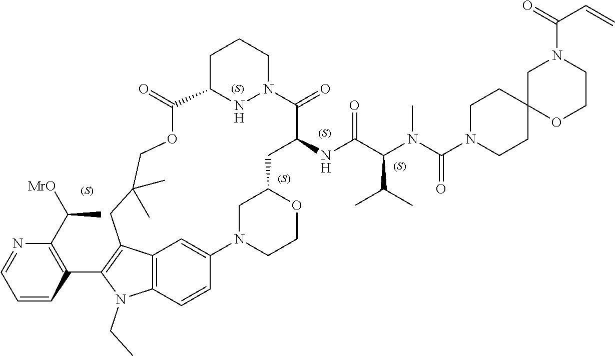 |
| BA 565 | 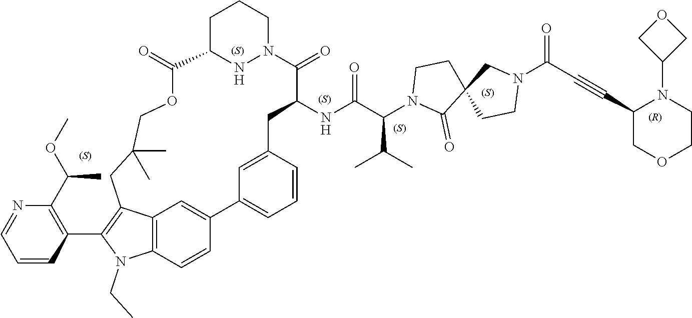 |
| BA 566 | 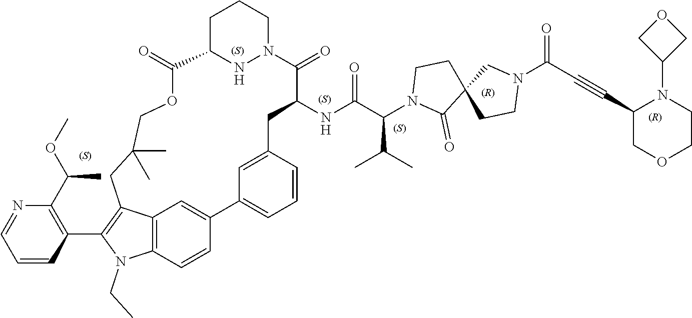 |
| BA 567 | 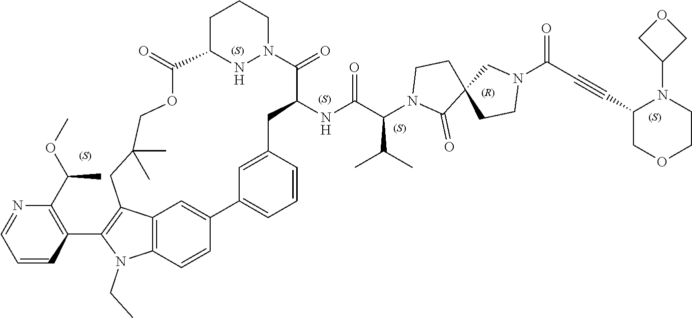 |
| BA 568 | 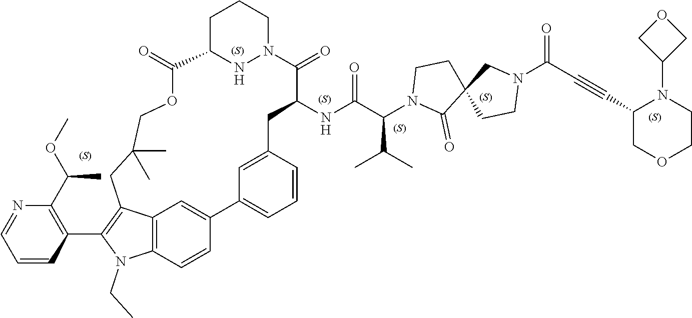 |
| BA 569 | 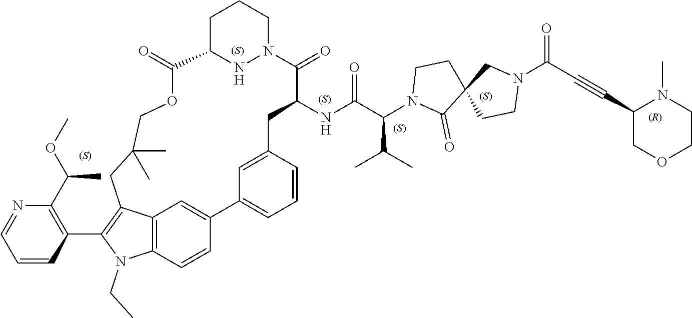 |
| BA 570 | 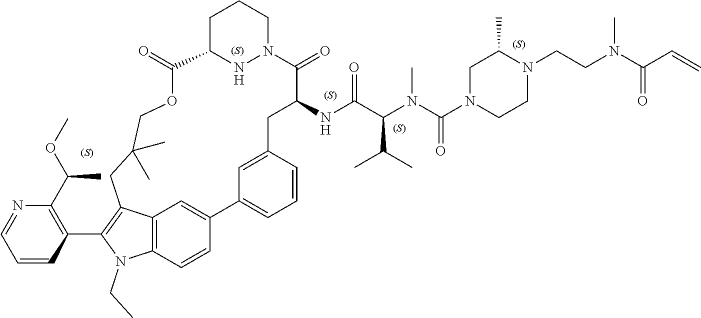 |
| BA 571 | 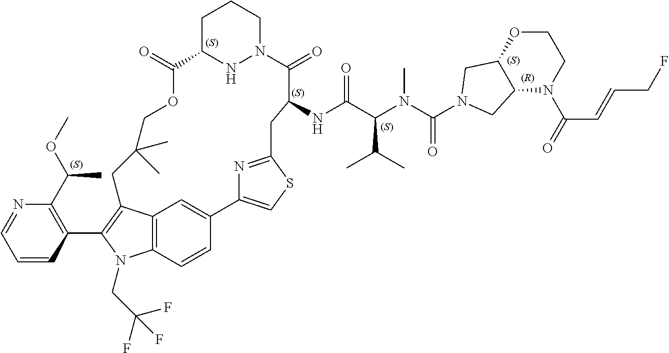 |
| BA 572 | 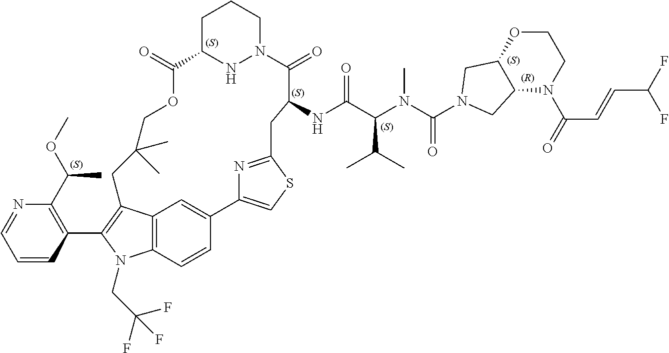 |
| BA 573 | 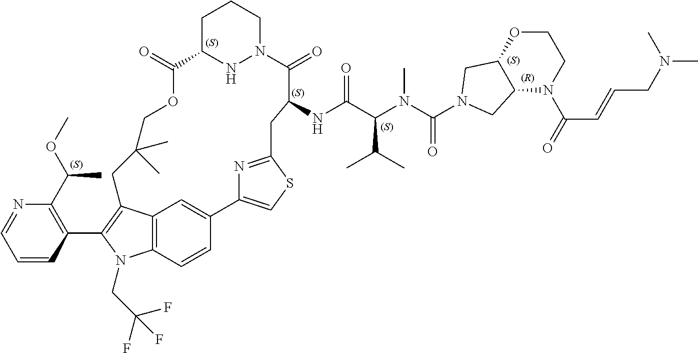 |
| BA 574 | 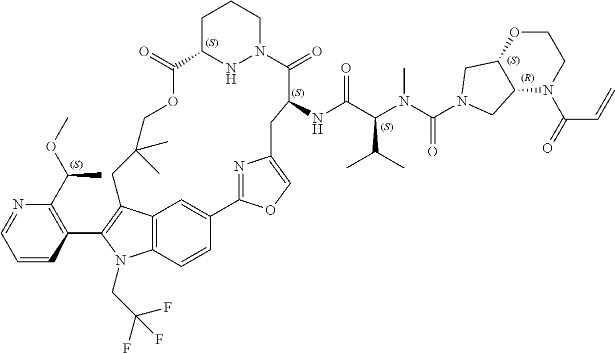 |
| BA 575 | 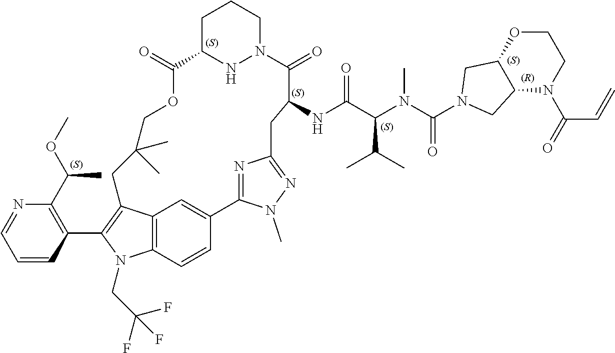 |
| BA 576 | 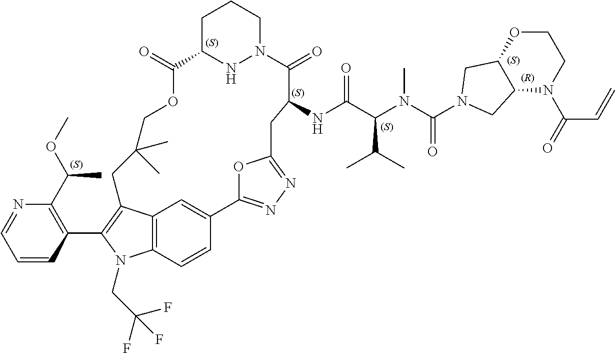 |
| BA 577 | 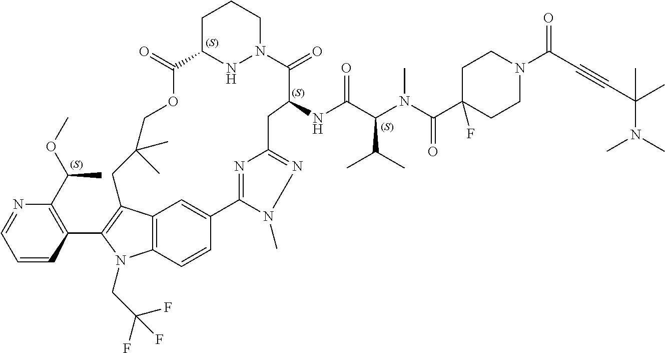 |
| BA 578 | 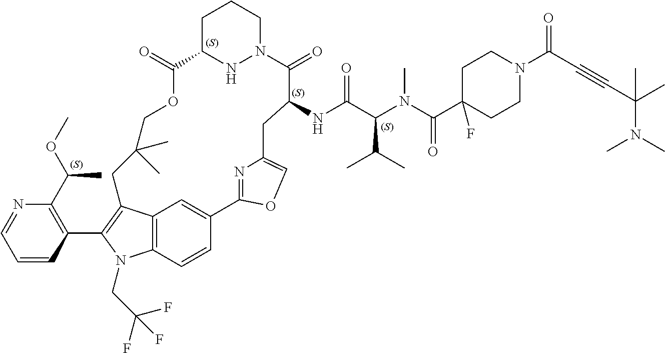 |
| BA 579 | 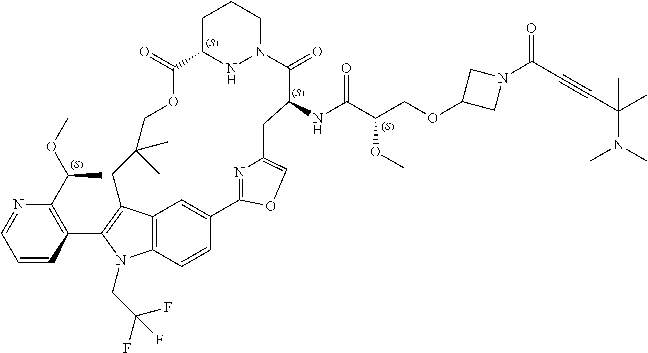 |
| BA 580 | 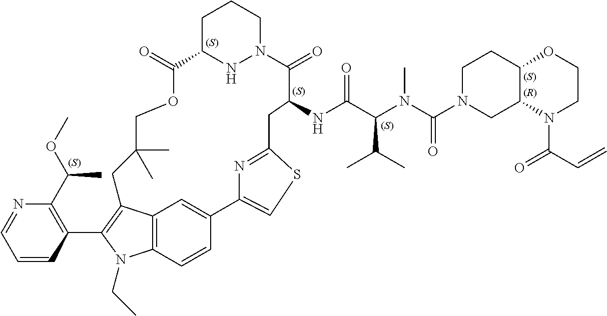 |
| BA 581 | 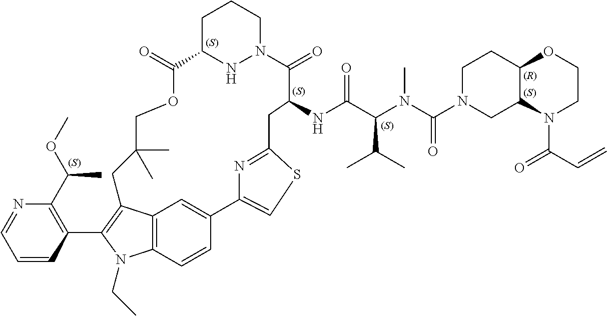 |
| BA 582 | 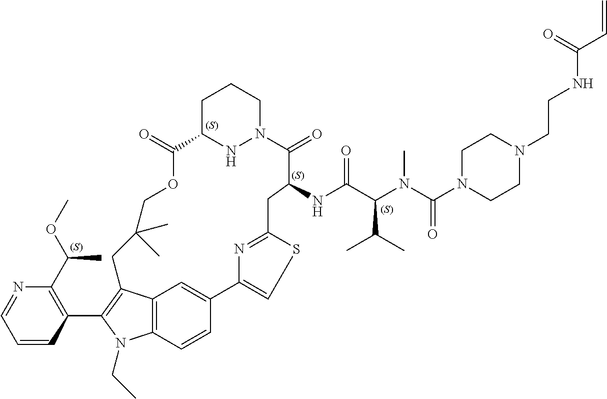 |
| BA 583 | 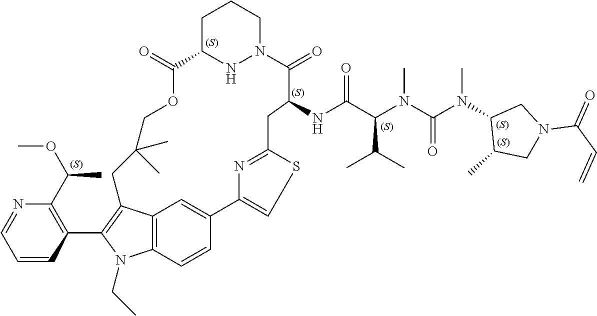 |
| BA 584 | 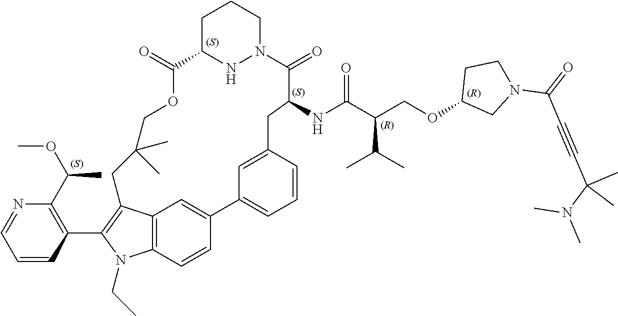 |
| BA 585 | 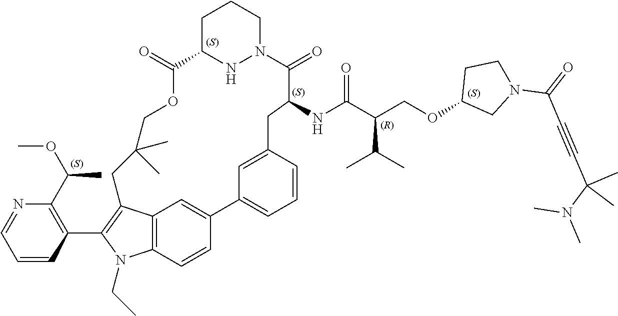 |
| BA 586 | 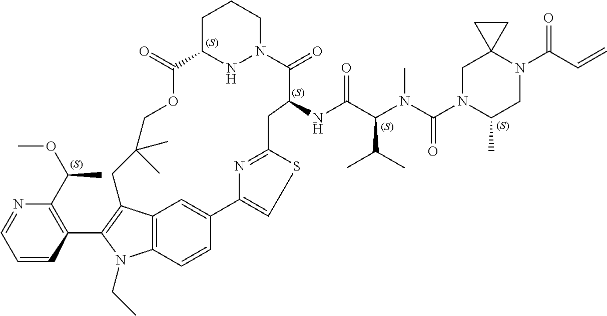 |
| BA 587 | 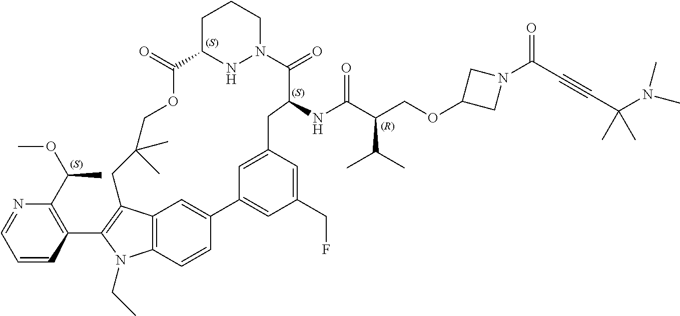 |
| BA 588 | 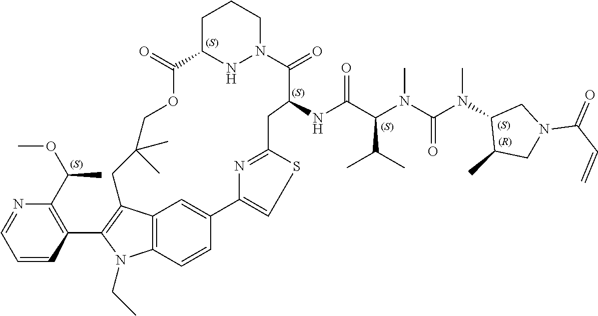 |
| BA 589 | 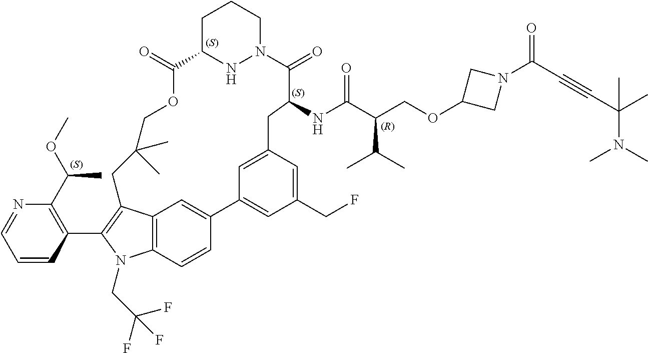 |
| BA 590 | 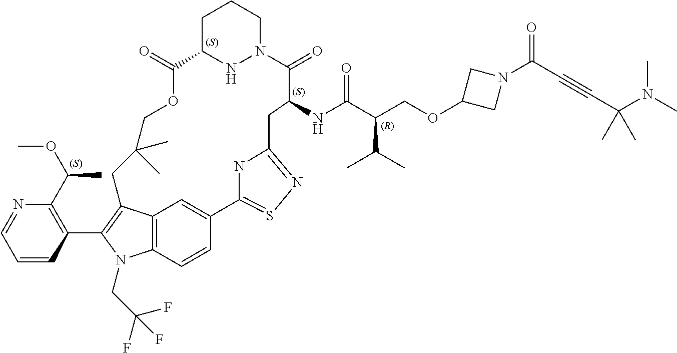 |
| BA 591 | 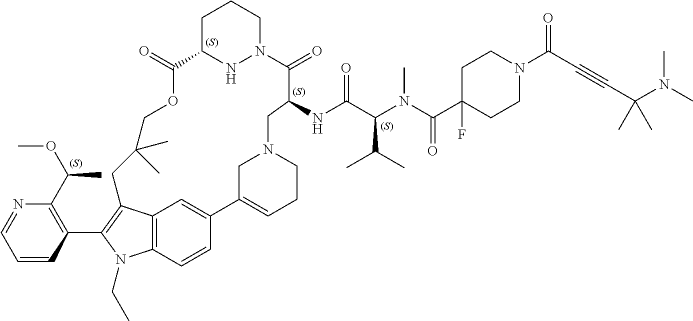 |
| BA 592 | 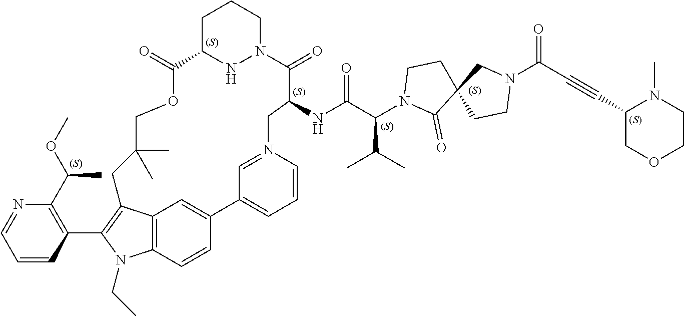 |
| BA 593 | 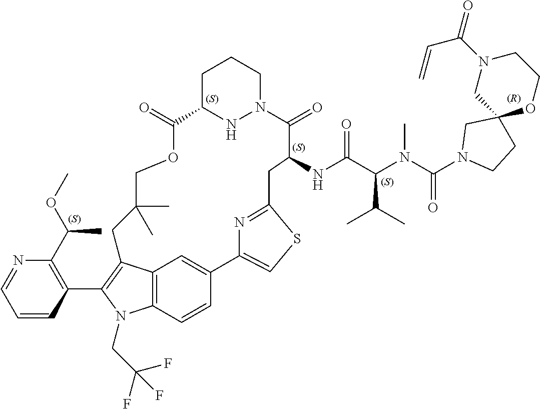 |
| BA 594 | 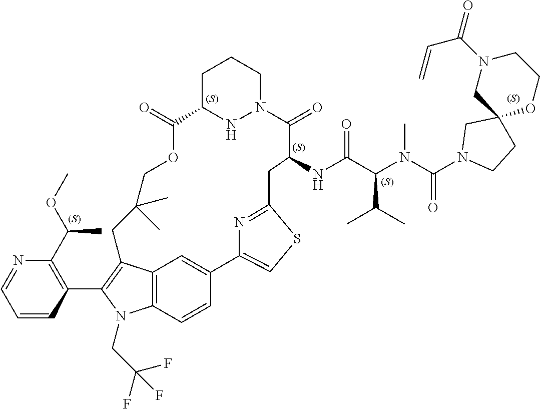 |
| BA 595 | 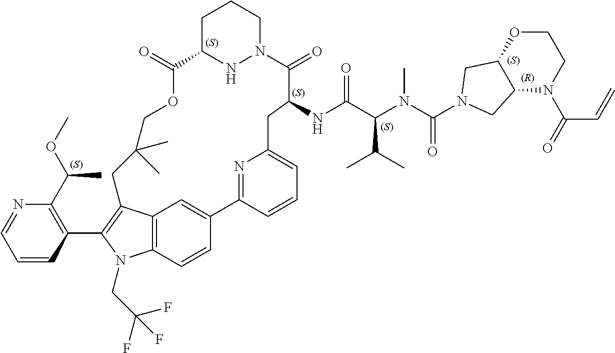 |
| BA 596 | 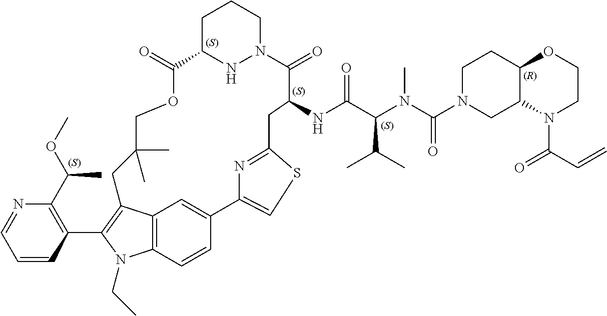 |
| BA 597 | 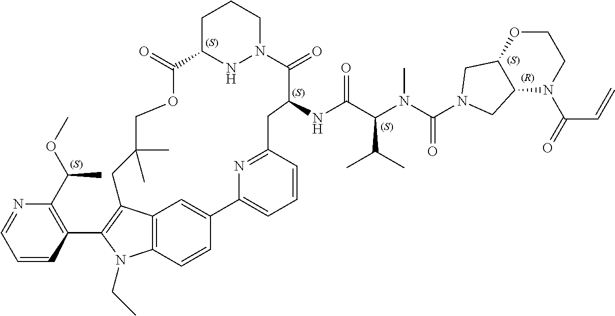 |
| BA 598 | 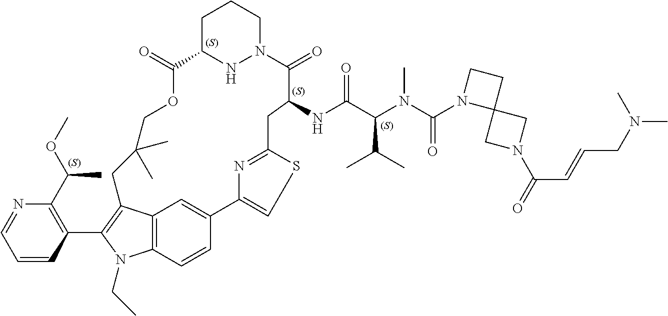 |
| BA 599 | 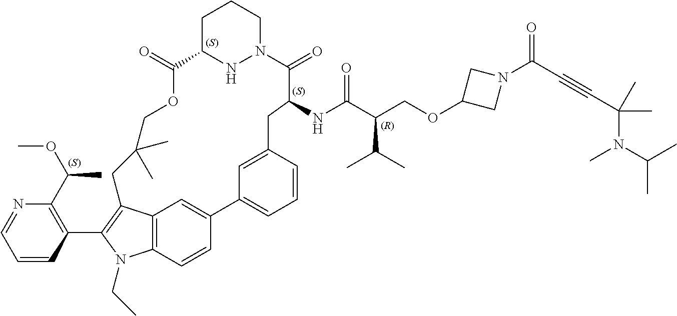 |
| BA 600 | 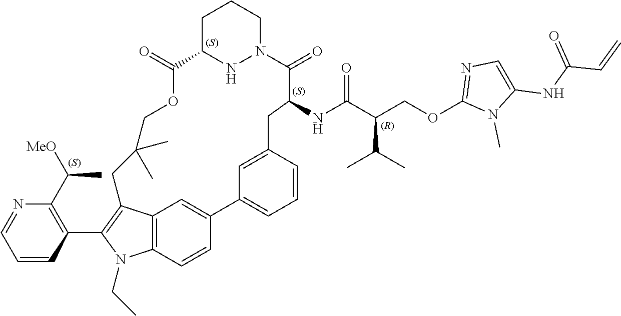 |
| BA 601 | 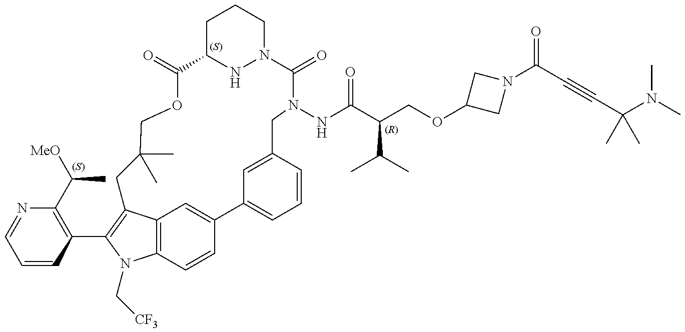 |
| BA 602 | 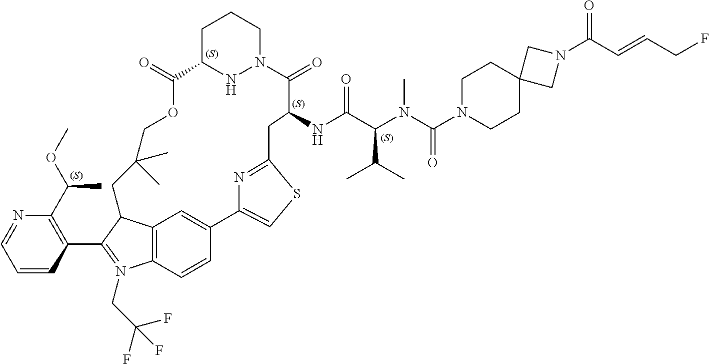 |
| BA 603 | 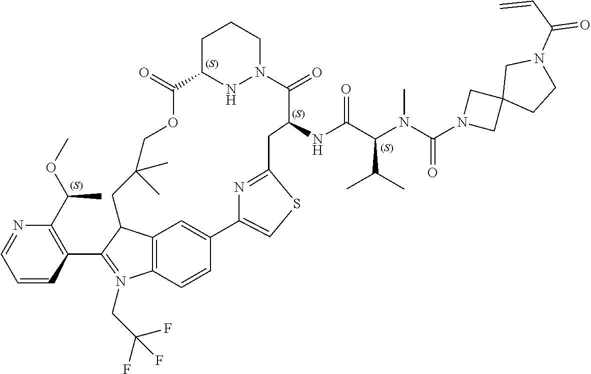 |
| BA 604 | 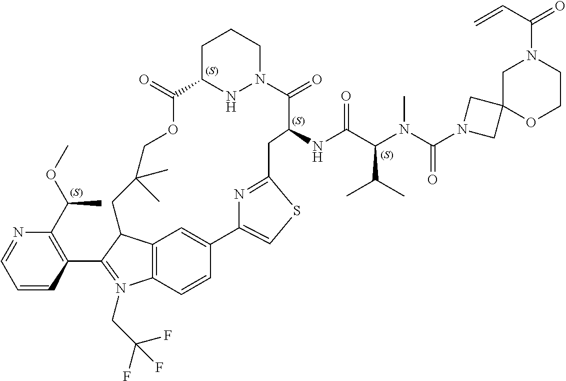 |
| BA 605 | 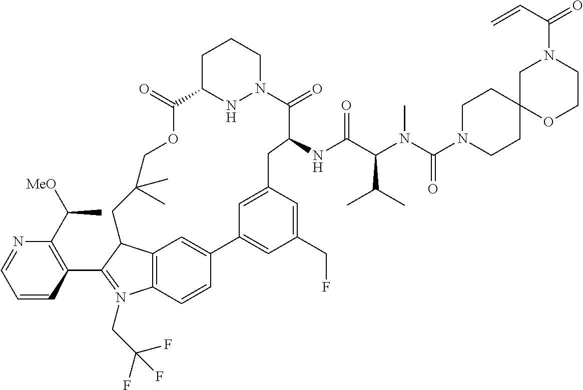 |
| BA 606 | 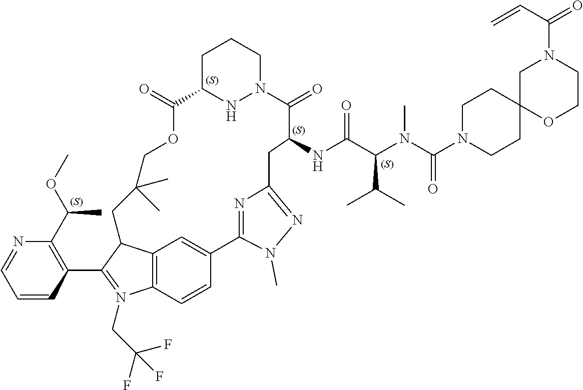 |
| BA 607 | 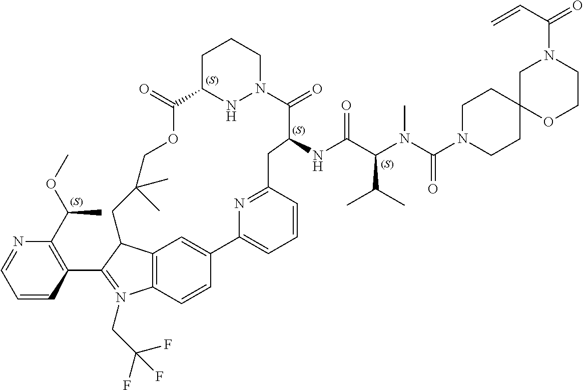 |
| BA 608 | 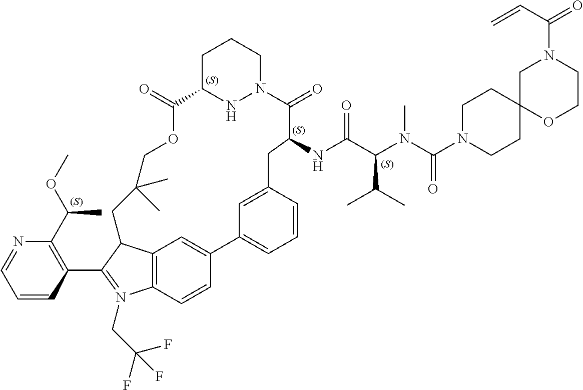 |
| BA 609 | 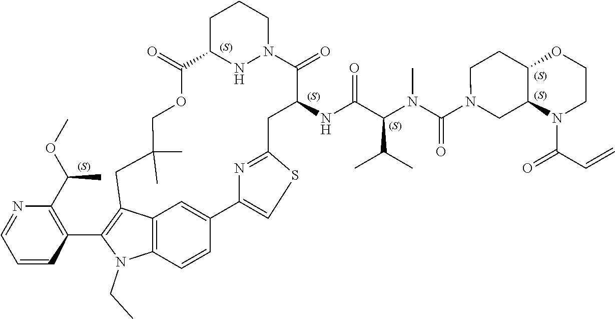 |
| BA 610 | 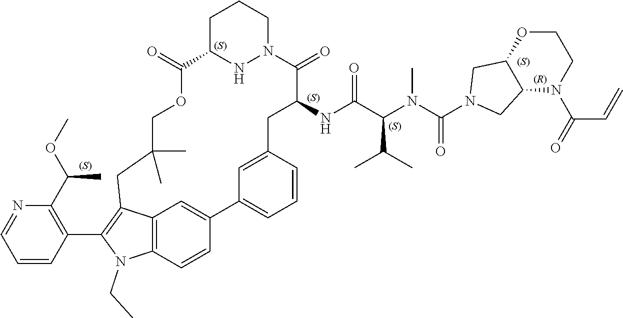 |
| BA 611 | 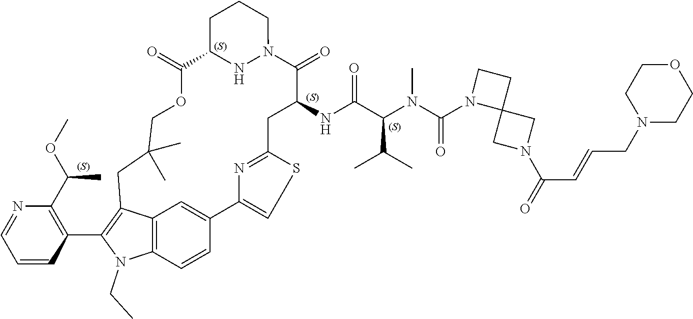 |
| BA 612 | 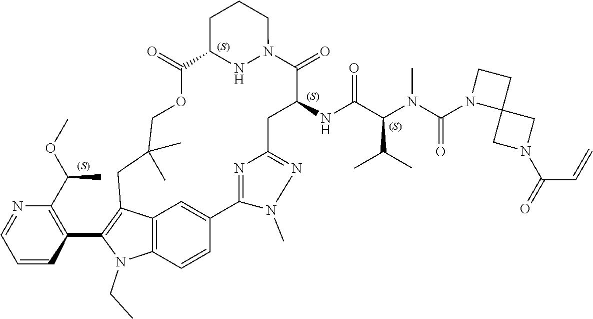 |
| BA 613 | 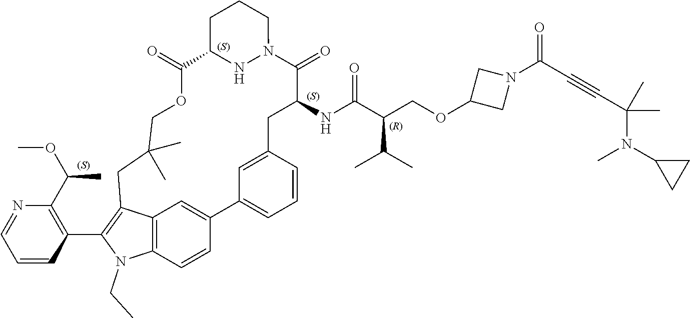 |
| BA 614 | 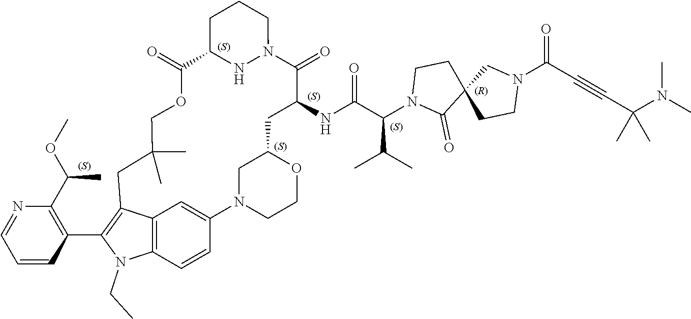 |
| BA 615 | 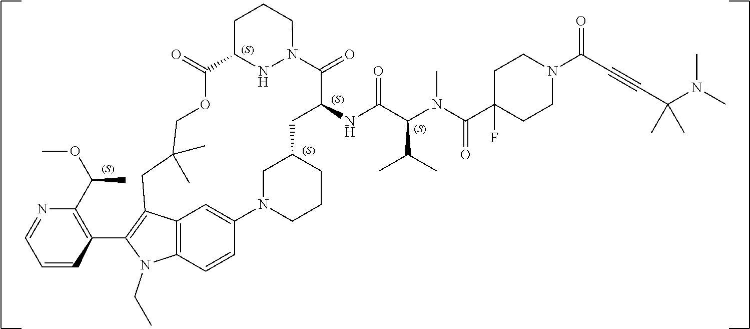 |
| BA 616 | 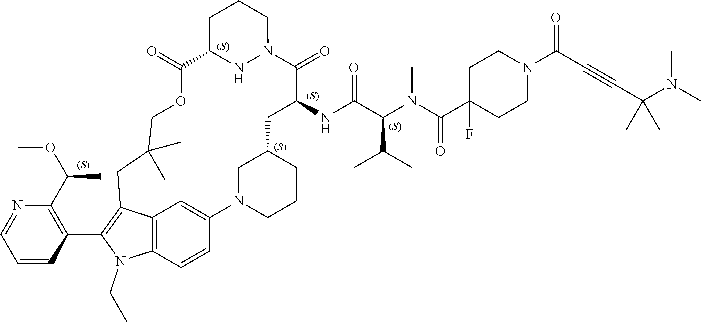 |
| BA 617 | 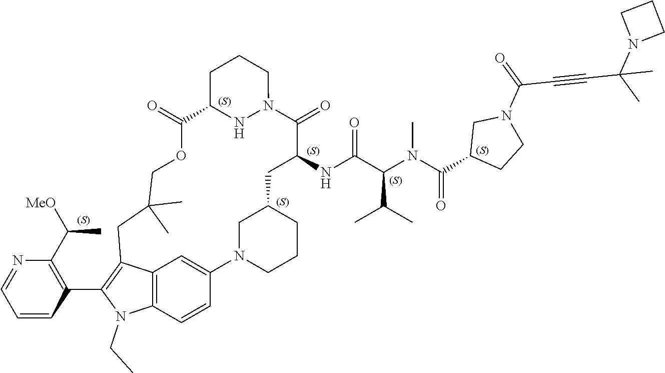 |
| BA 618 | 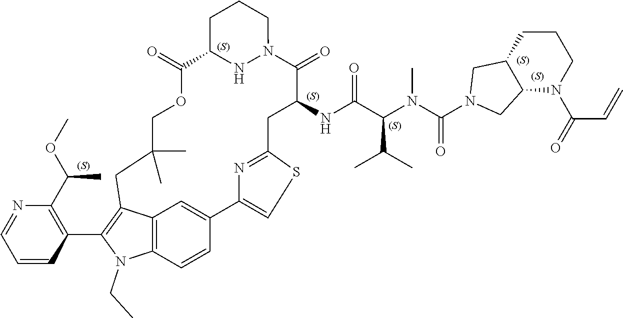 |
| BA 619 | 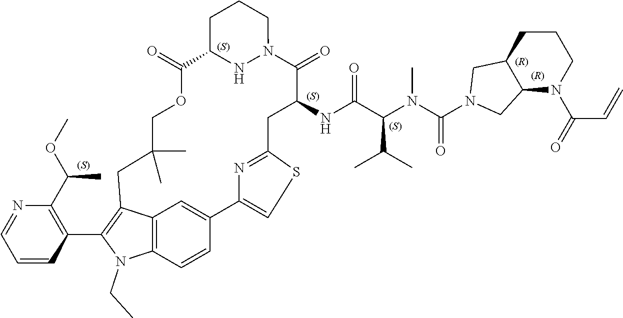 |
| BA 620 | 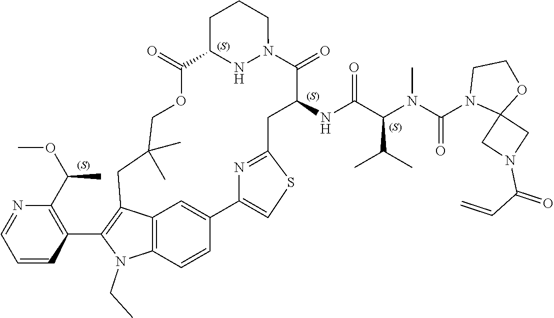 |
| BA 621 | 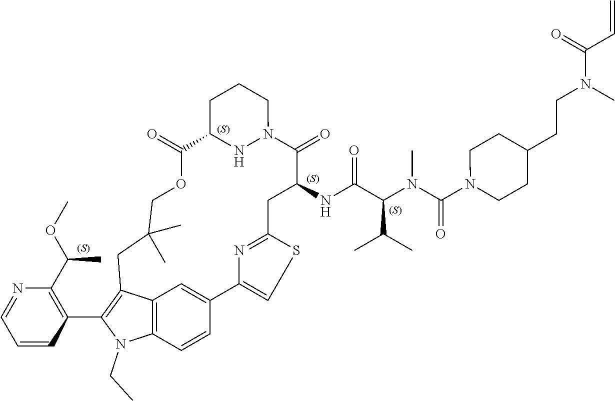 |
| BA 622 | 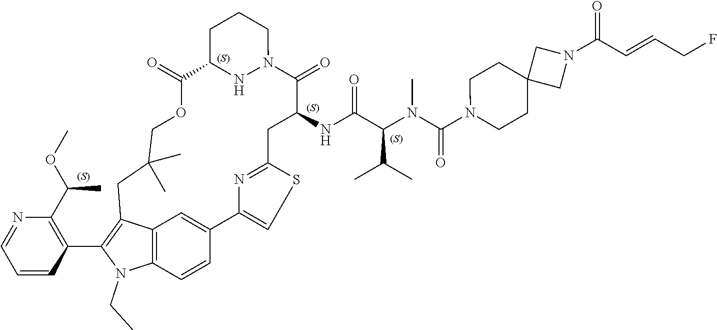 |
| BA 623 | 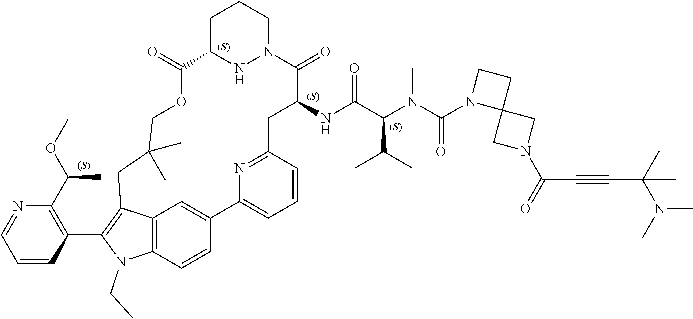 |
| BA 624 | 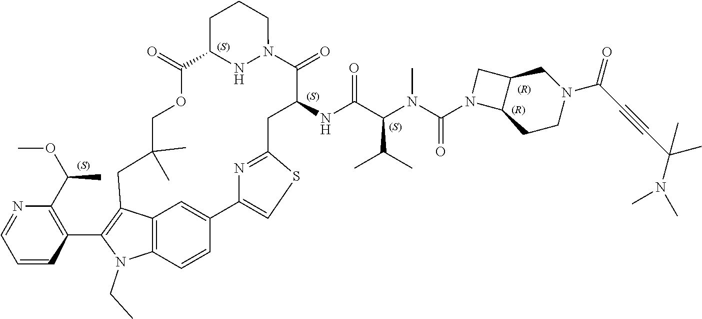 |
| BA 625 | 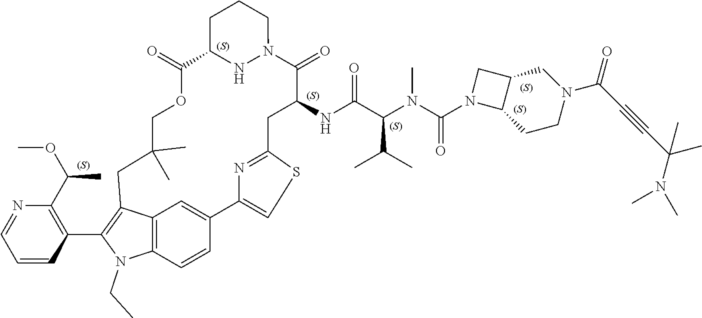 |
| BA 626 | 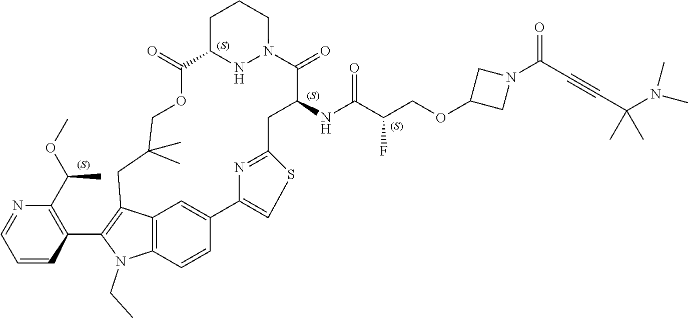 |
| BA 627 | 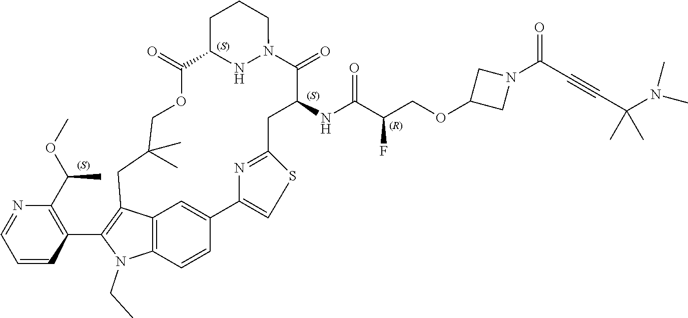 |
| BA 628 | 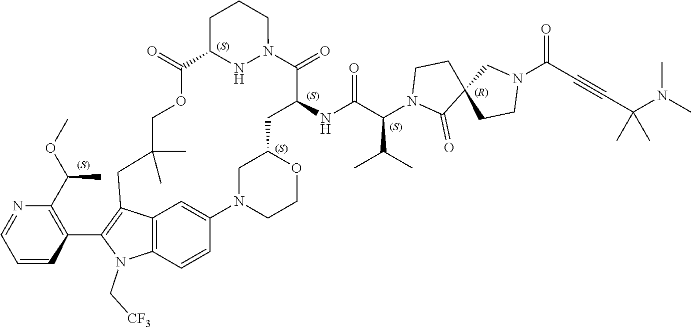 |
| BA 629 | 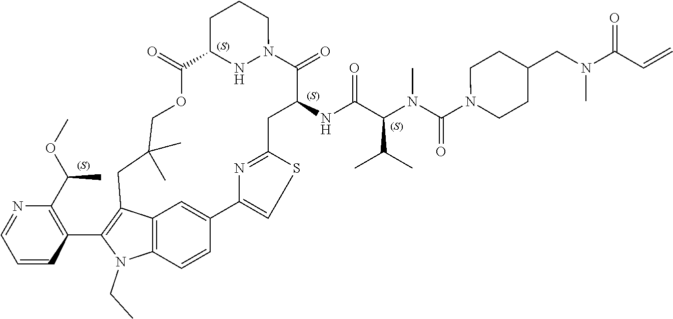 |
| BA 630 | 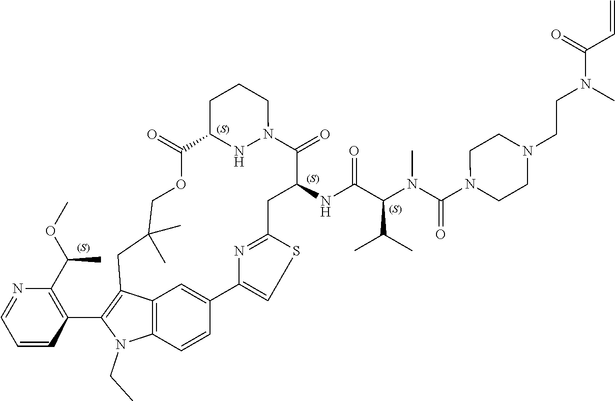 |
| BA 631 | 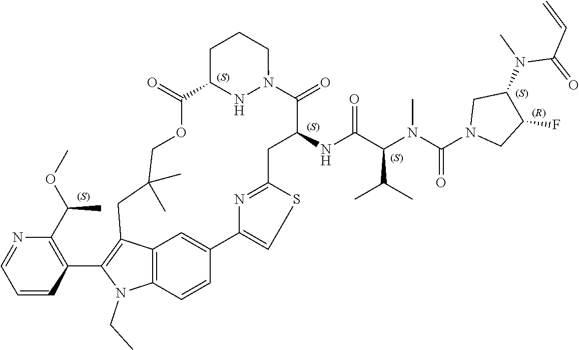 |
| BA 632 | 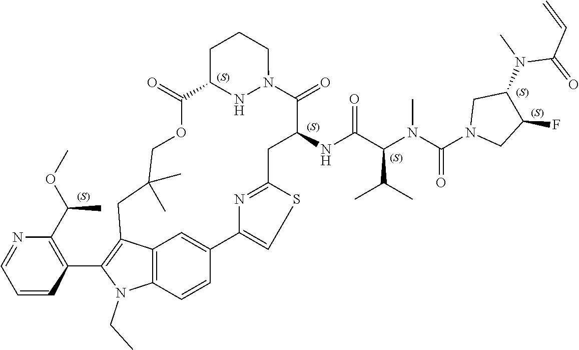 |
| BA 633 | 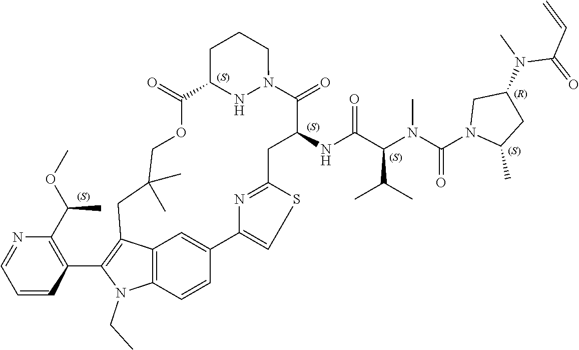 |
| BA 634 | 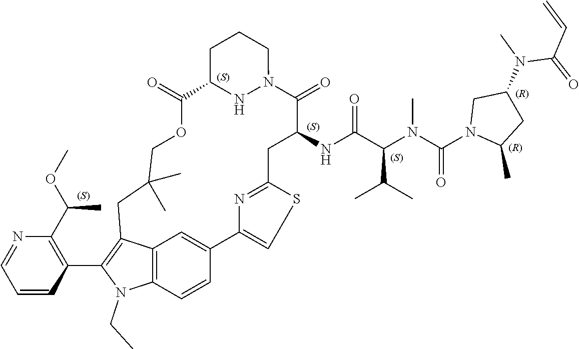 |
| BA 635 | 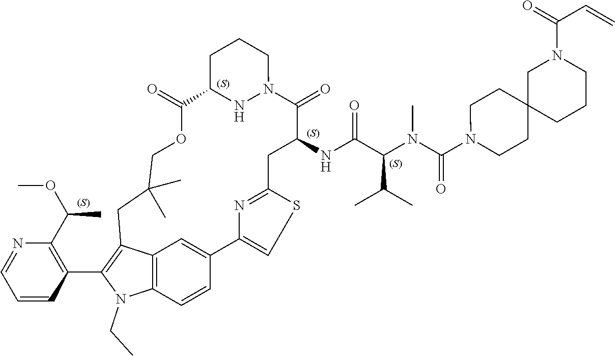 |
| BA 636 | 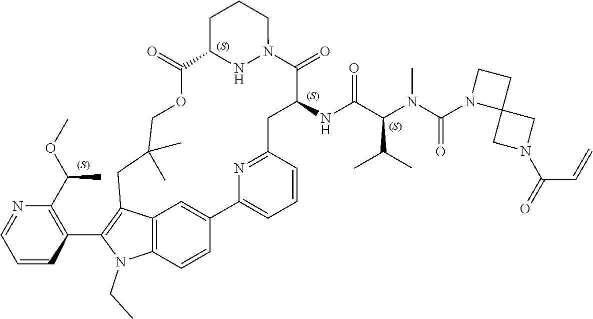 |
| BA 637 | 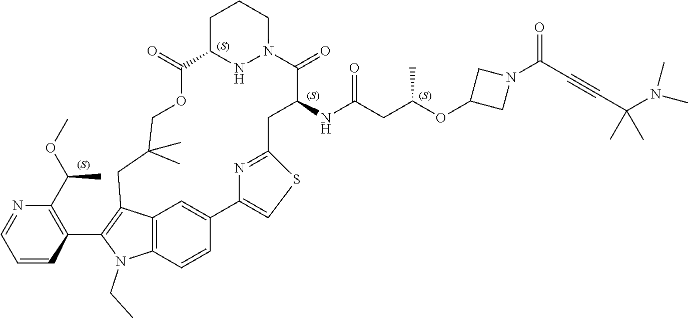 |
| BA 638 | 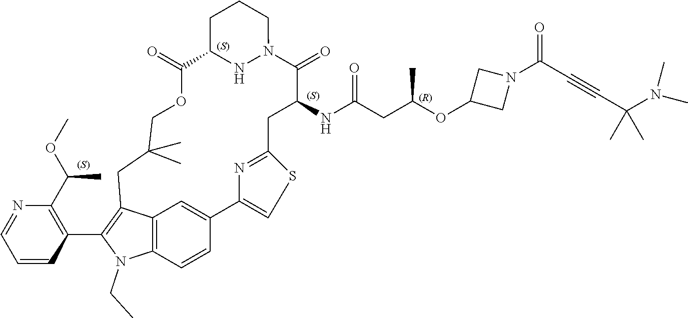 |
| BA 639 | 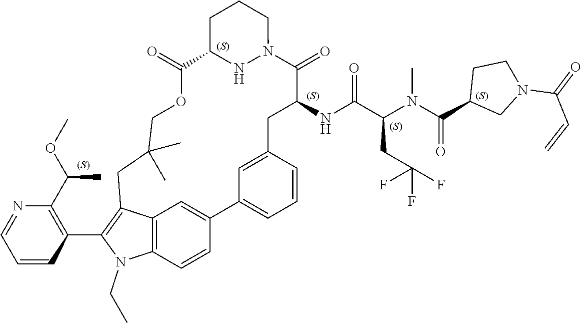 |
| BA 640 | 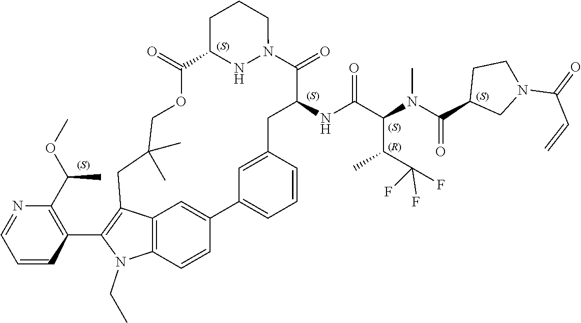 |
| BA 641 | 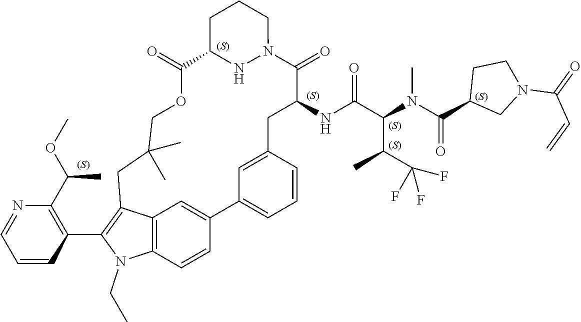 |
| BA 642 | 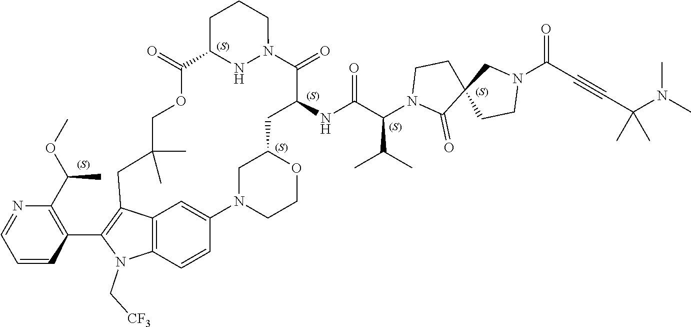 |
| BA 643 | 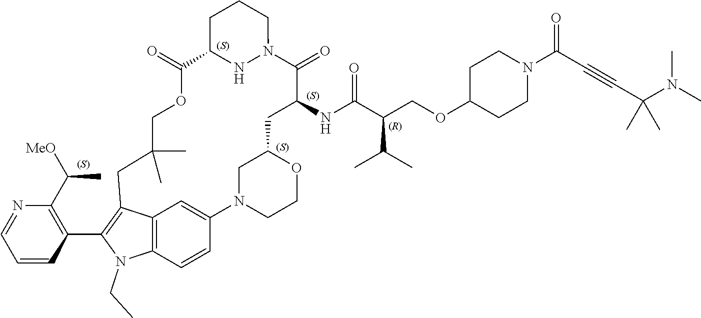 |
| BA 644 | 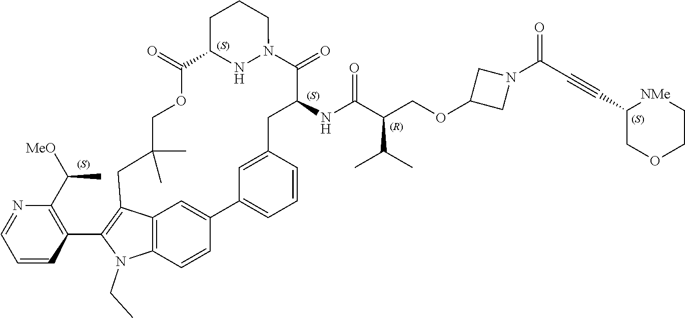 |
| BA 645 | 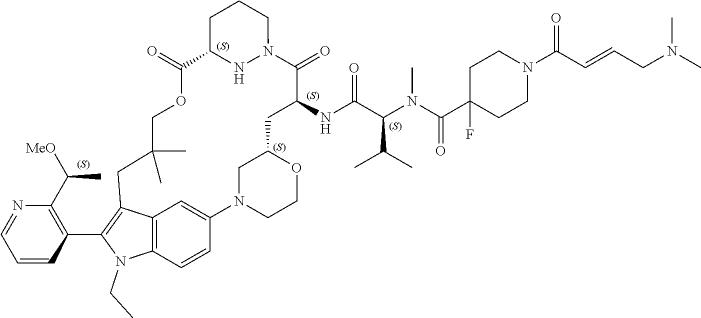 |
| BA 646 | 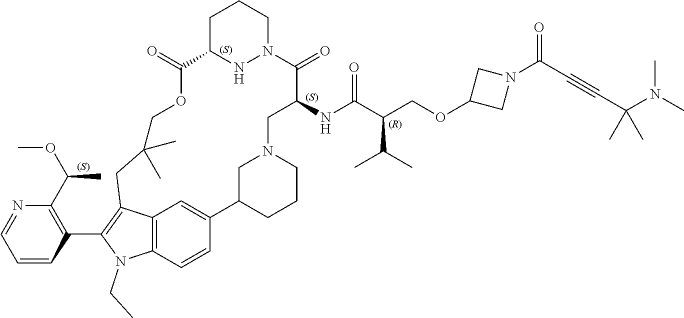 |
| BA 647 | 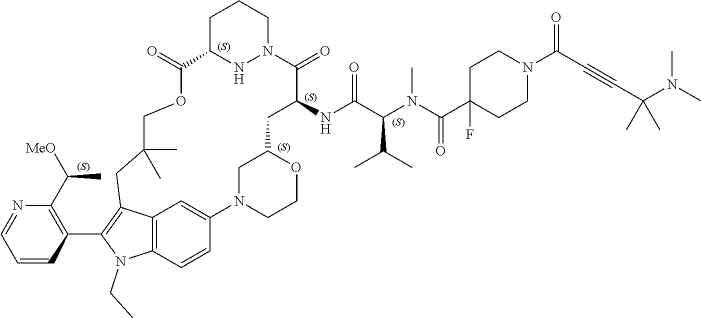 |
| BA 648 | 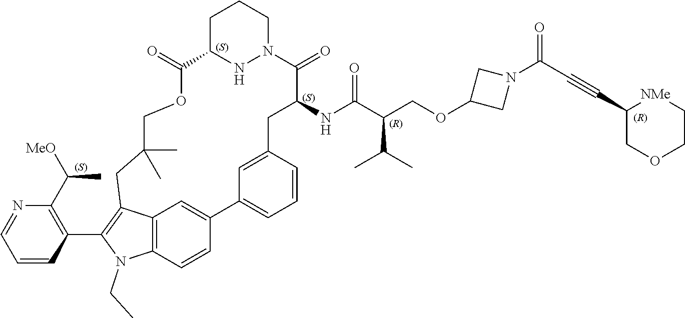 |
| BA 649 | 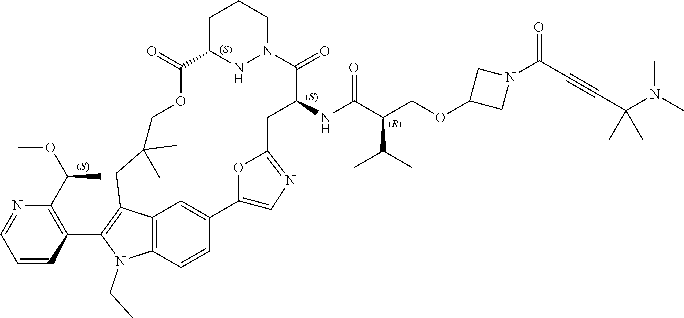 |
| BA 650 | 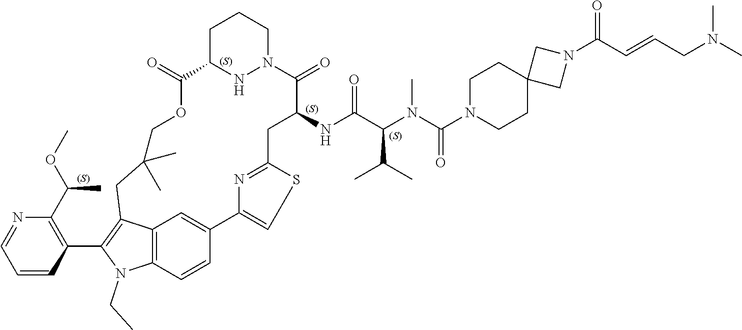 |
| BA 651 | 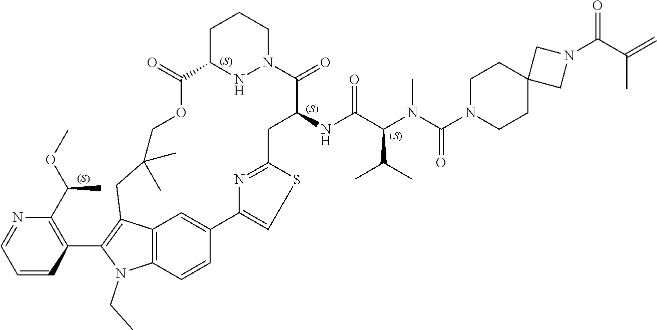 |
| BA 652 | 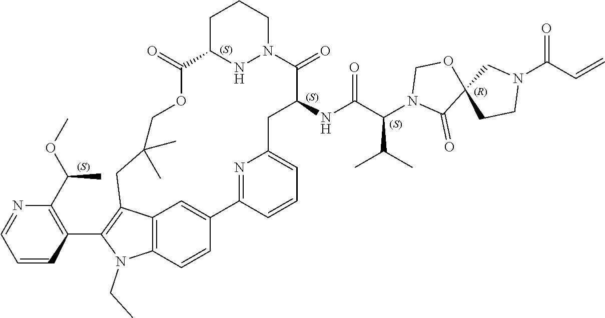 |
| BA 653 | 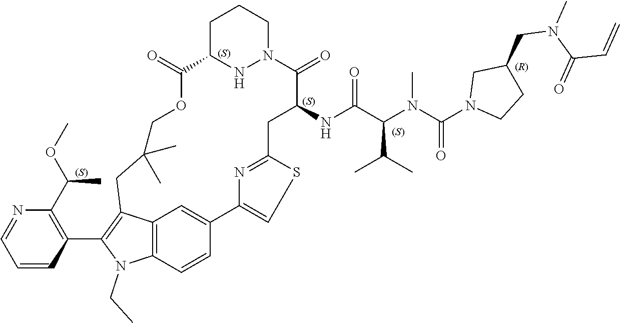 |
| BA 654 | 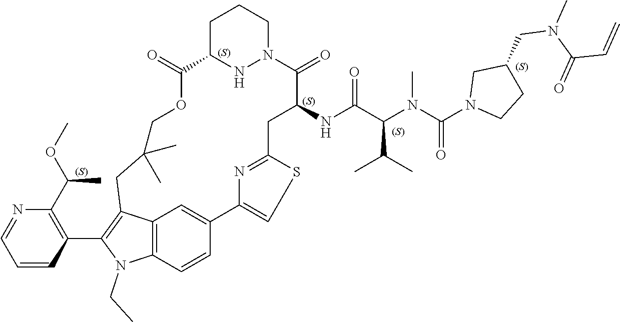 |
| BA 655 | 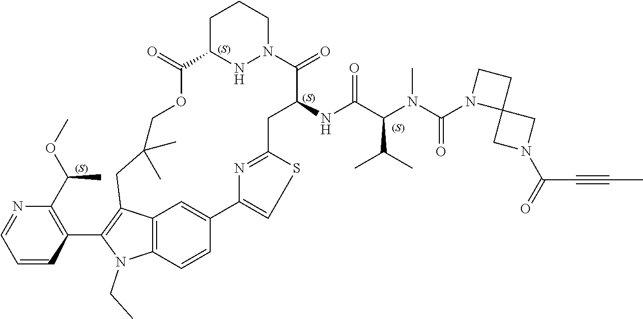 |
| BA 656 | 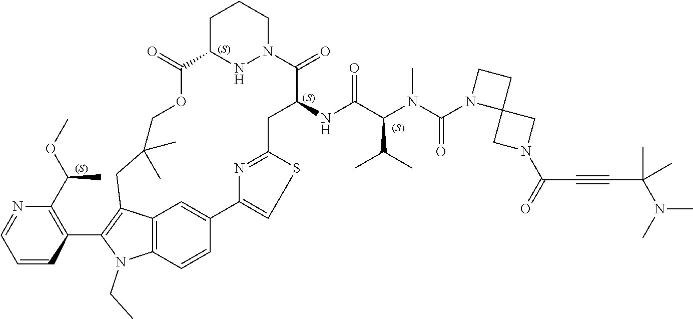 |
| BA 657 | 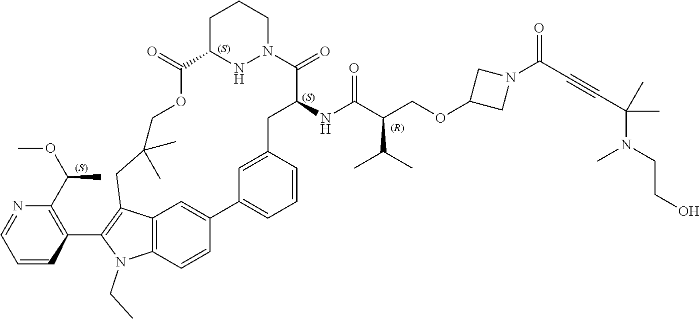 |
| BA 658 | 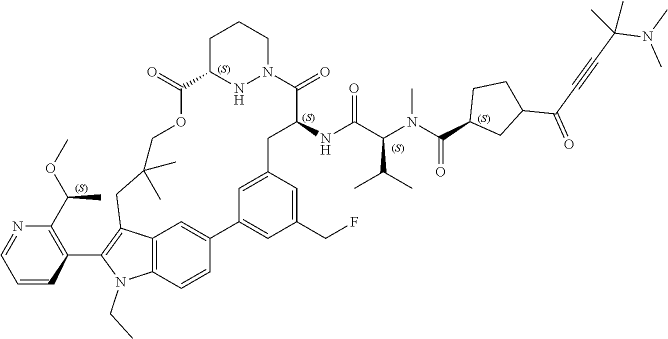 |
| BA 659 | 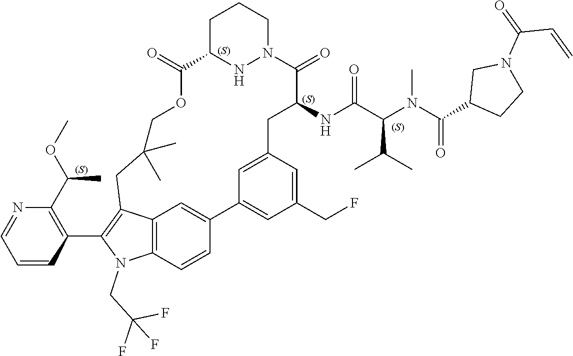 |
| BA 660 | 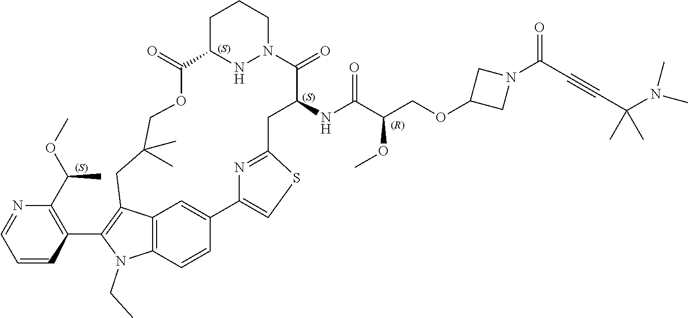 |
| BA 661 | 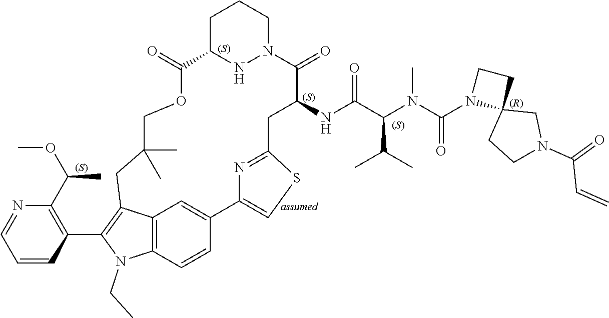 |
| BA 662 | 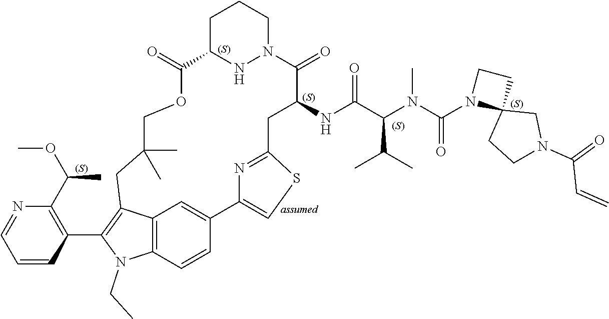 |
| BA 663 | 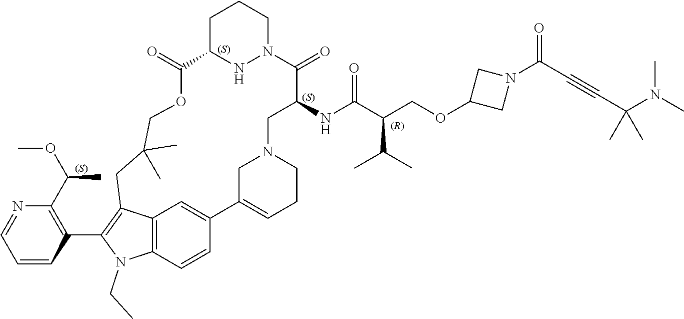 |
| BA 664 | 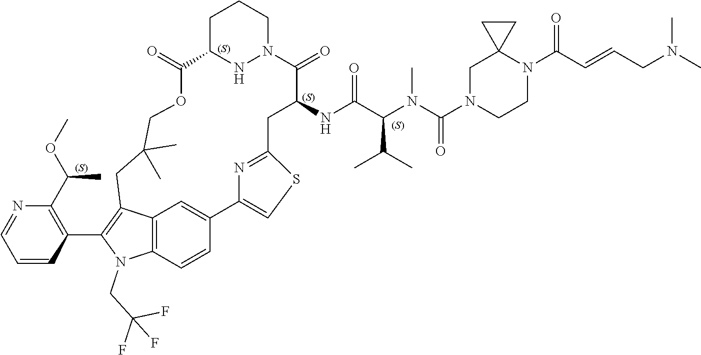 |
| BA 665 | 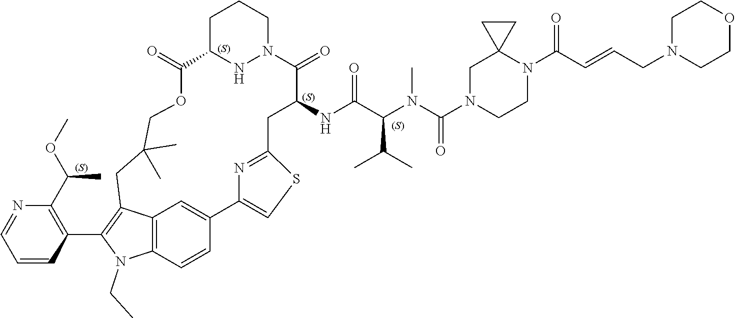 |
| BA 666 | 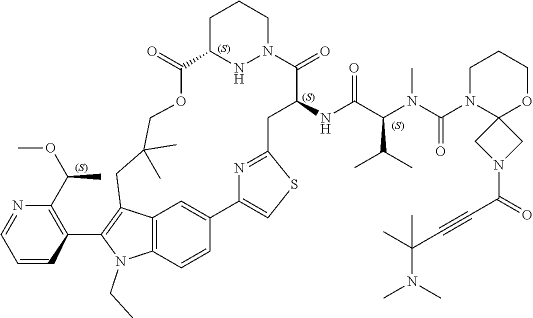 |
| BA 667 | 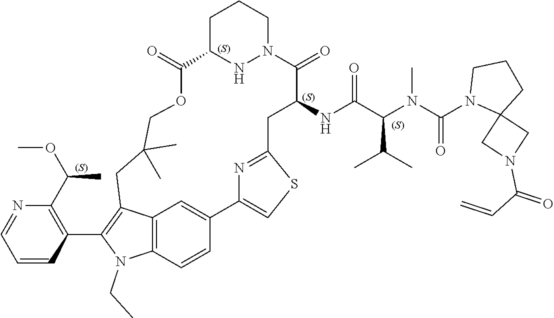 |
| BA 668 | 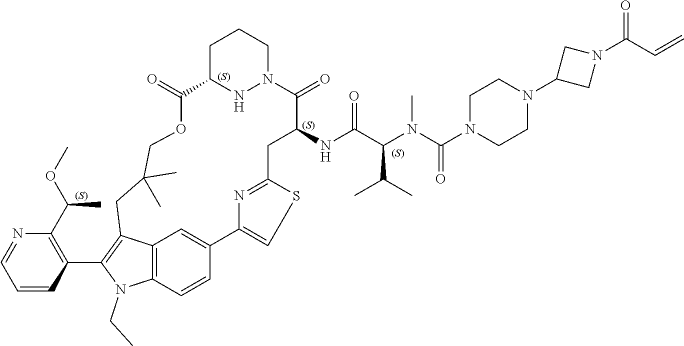 |
| BA 669 | 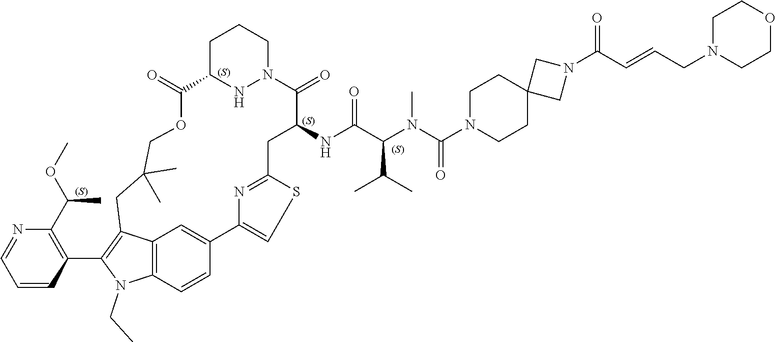 |
| BA 670 | 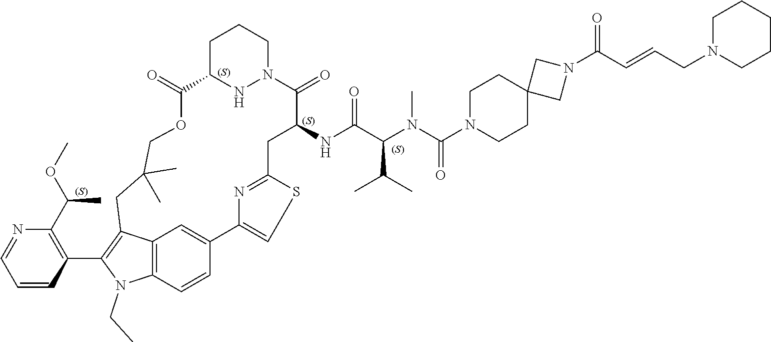 |
| BA 671 | 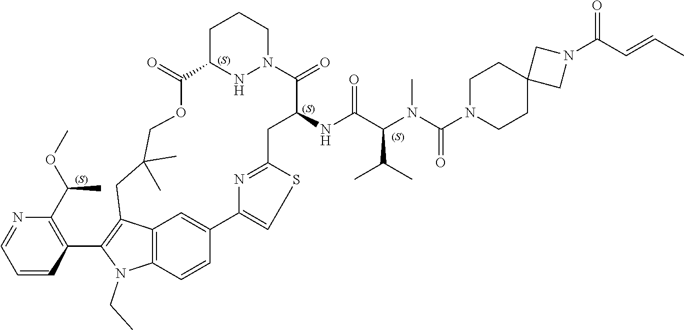 |
| BA 672 | 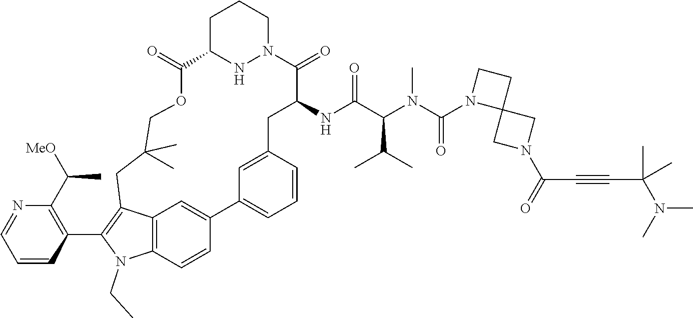 |
| BA 673 | 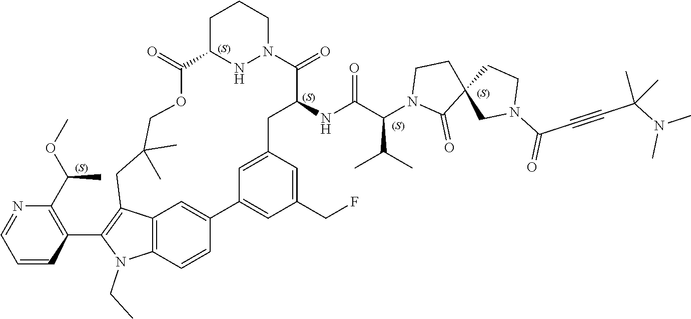 |
| BA 674 | 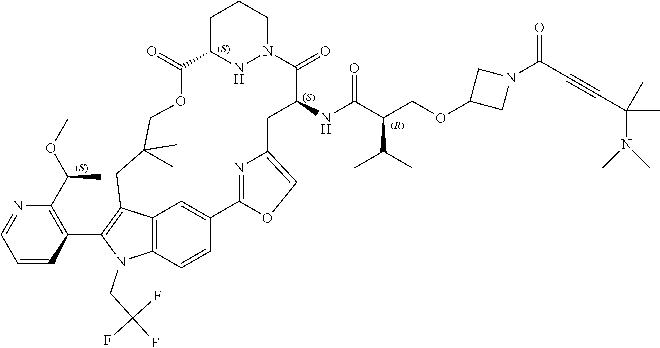 |
| BA 675 | 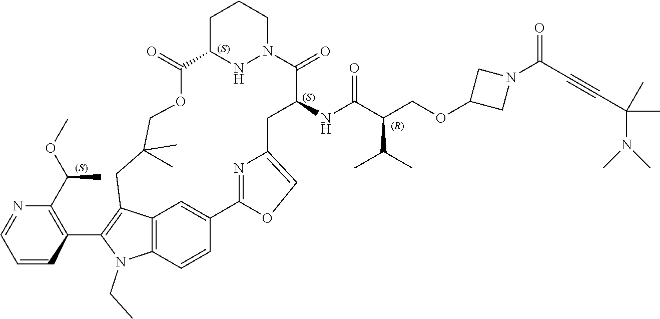 |
| BA 676 | 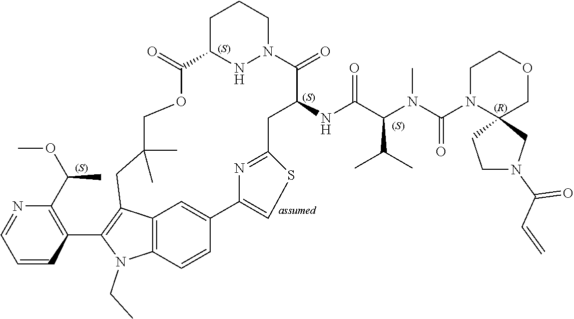 |
| BA 677 | 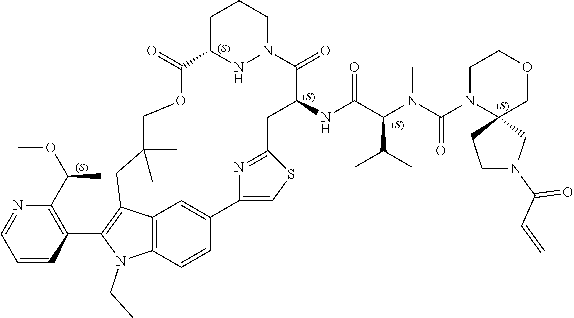 |
| BA 678 | 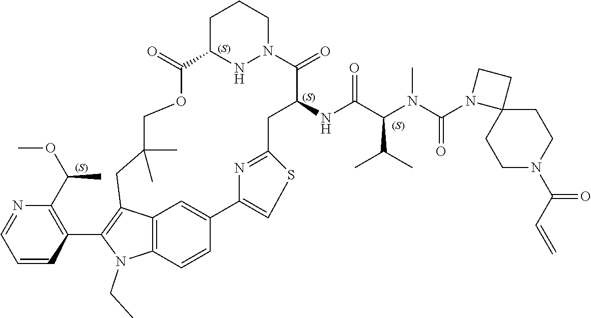 |
| BA 679 | 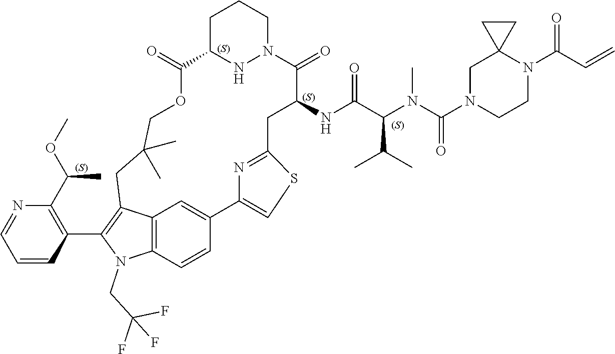 |
| BA 680 | 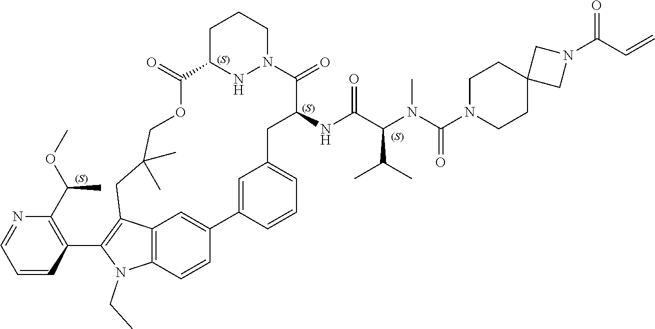 |
| BA 681 | 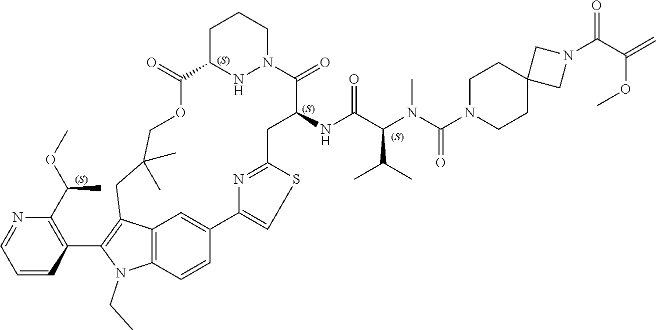 |
| BA 682 | 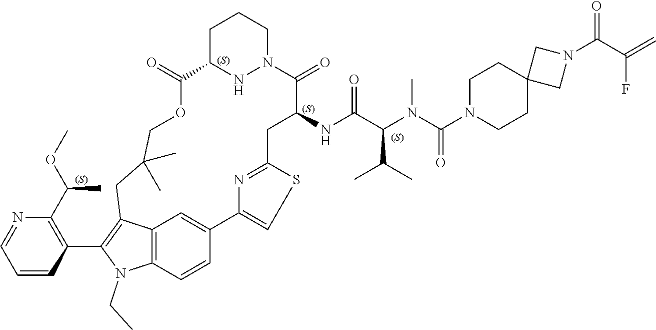 |
| BA 683 | 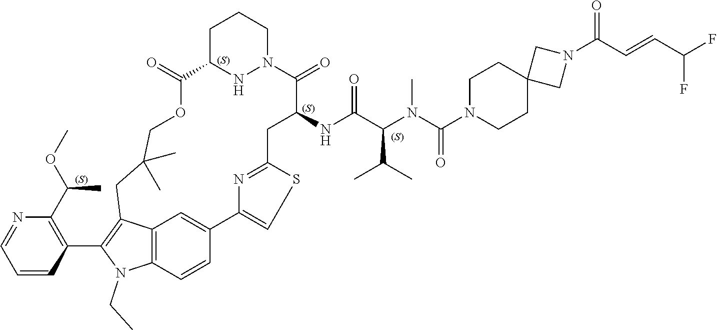 |
| BA 684 | 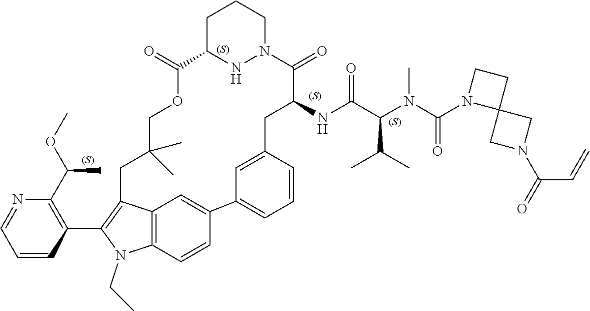 |
| BA 685 | 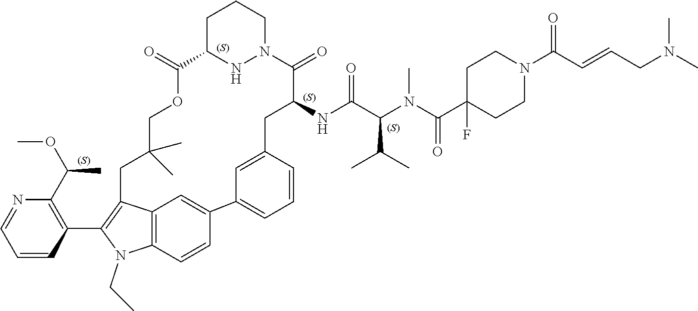 |
| BA 686 | 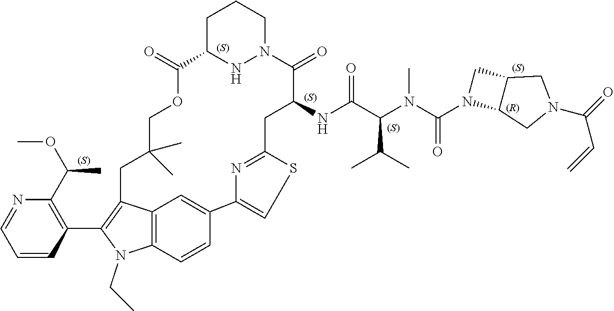 |
| BA 687 | 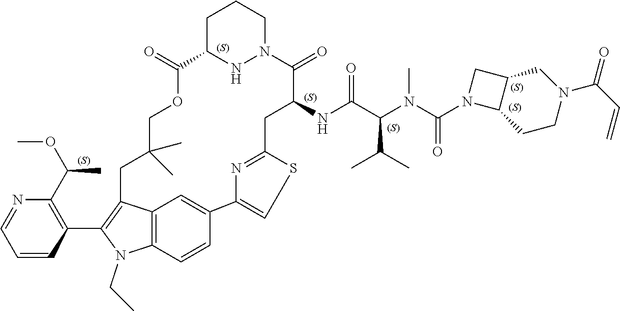 |
| BA 688 | 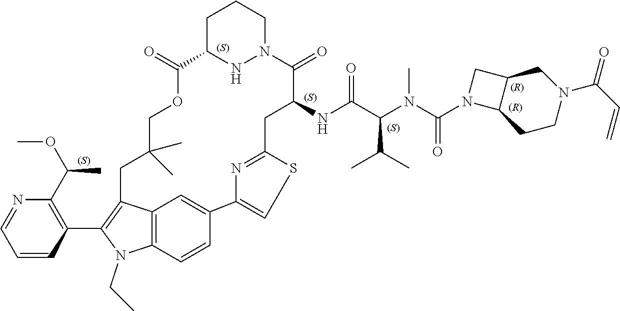 |
| BA 689 | 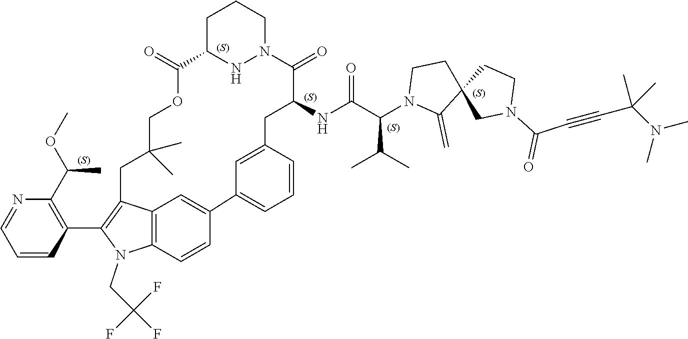 |
| BA 690 | 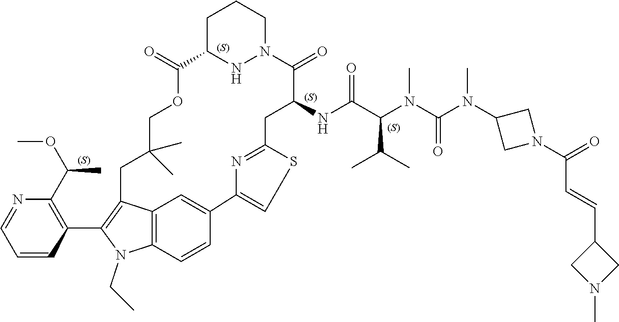 |
| BA 691 | 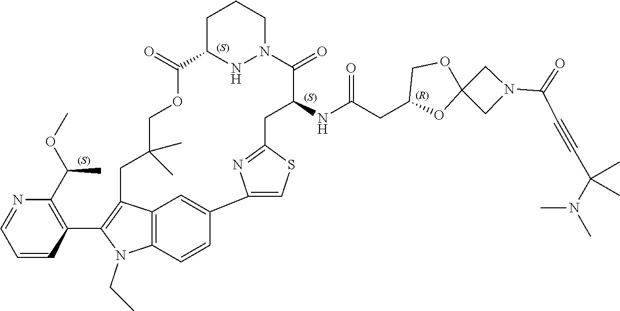 |
| BA 692 | 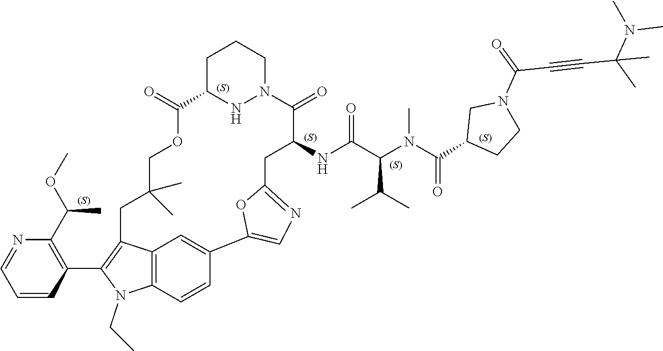 |
| BA 693 | 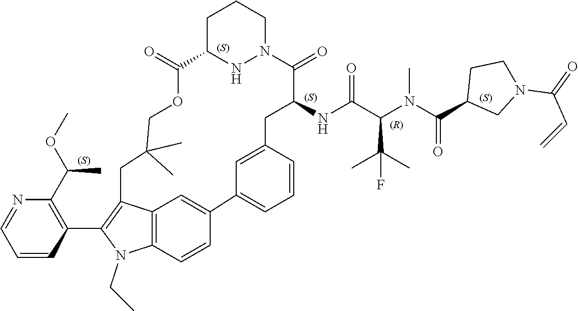 |
| BA 694 | 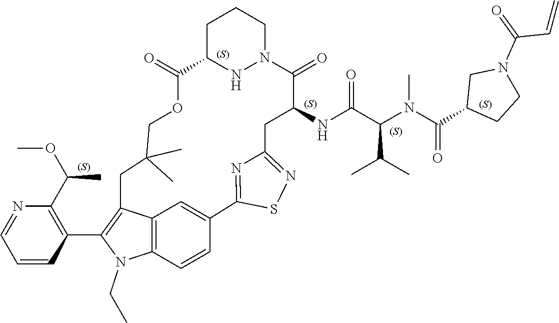 |
| BA 695 | 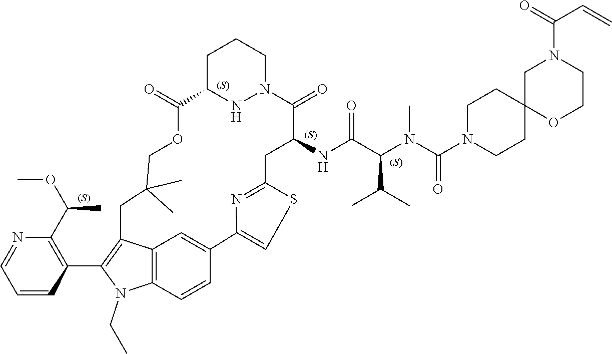 |
| BA 696 | 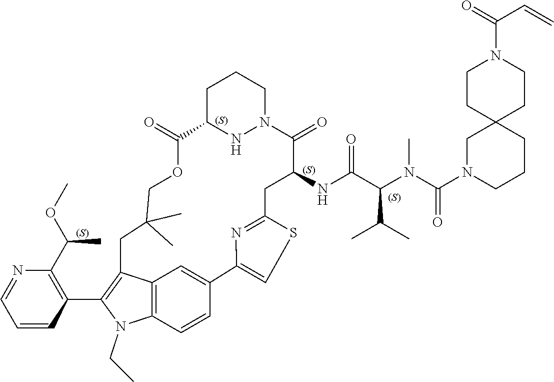 |
| BA 697 | 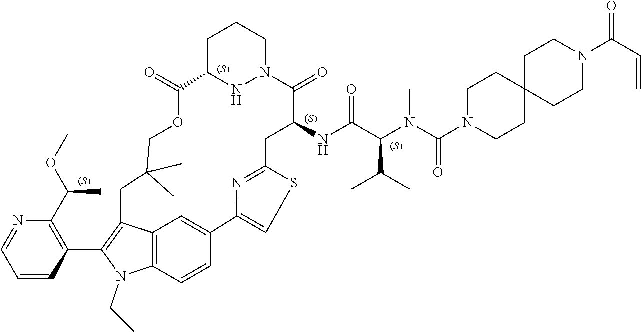 |
| BA 698 | 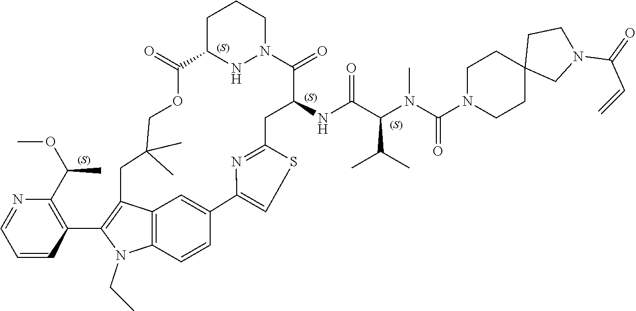 |
| BA 699 | 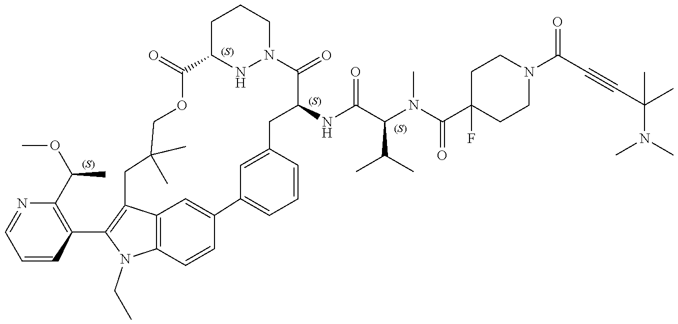 |
| BA 700 | 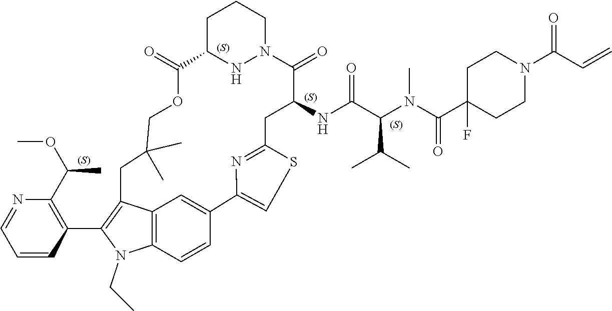 |
| BA 701 | 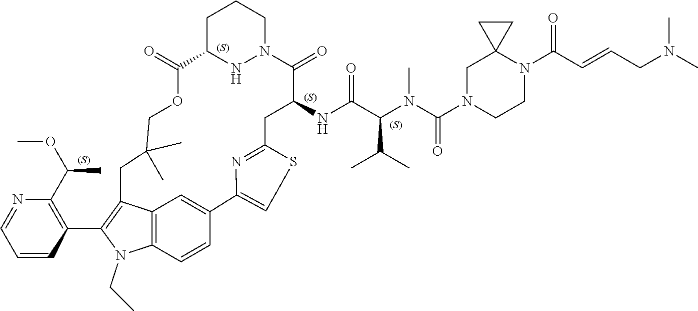 |
| BA 702 | 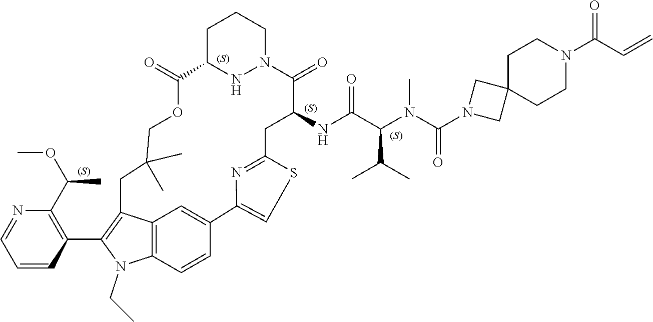 |
| BA 703 | 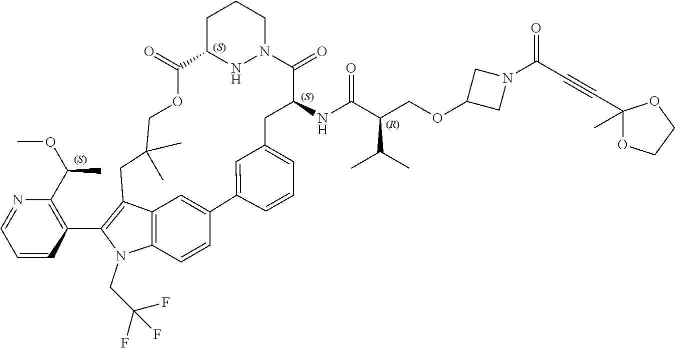 |
| BA 704 | 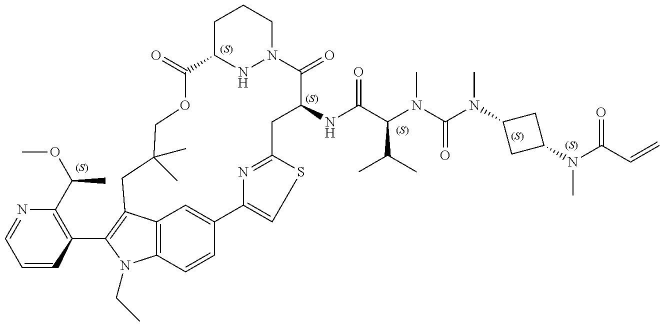 |
| BA 705 | 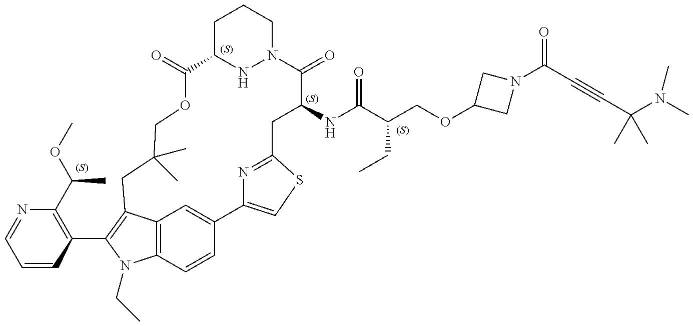 |
| BA 706 | 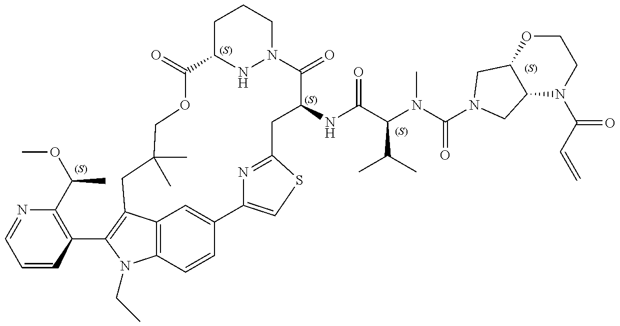 |
| BA 707 | 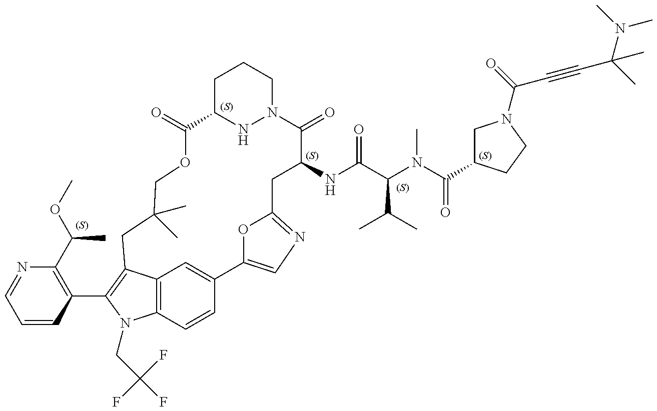 |
| BA 708 | 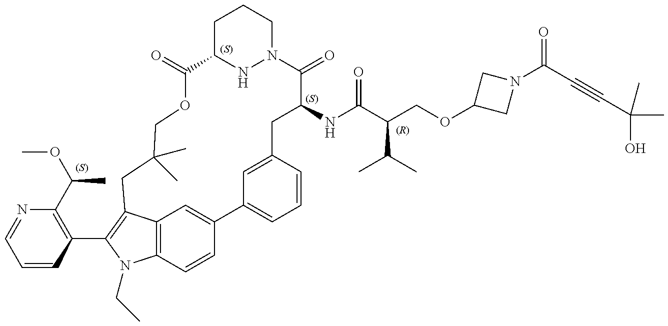 |
| BA 709 | 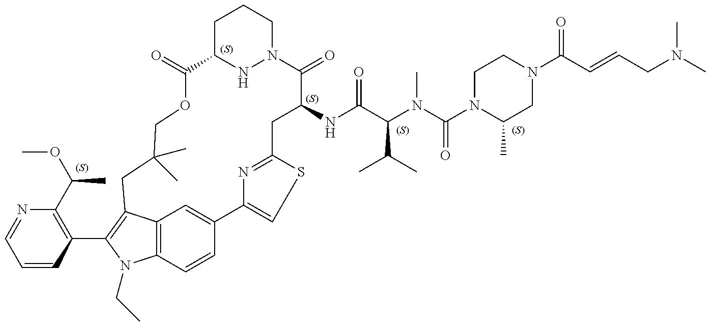 |
| BA 710 | 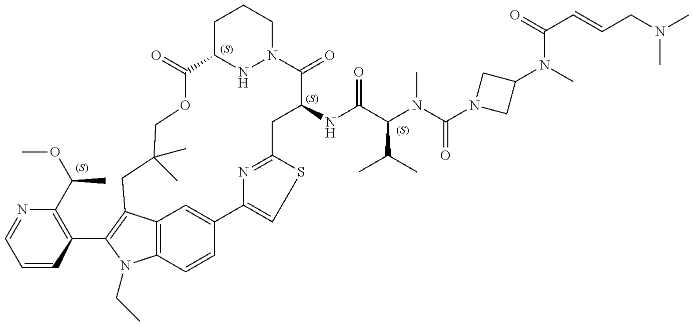 |
| BA 711 | 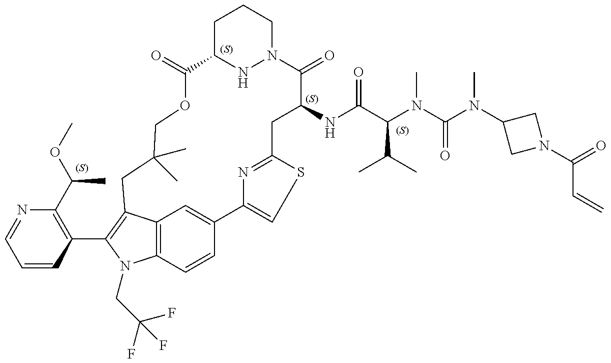 |
| BA 712 | 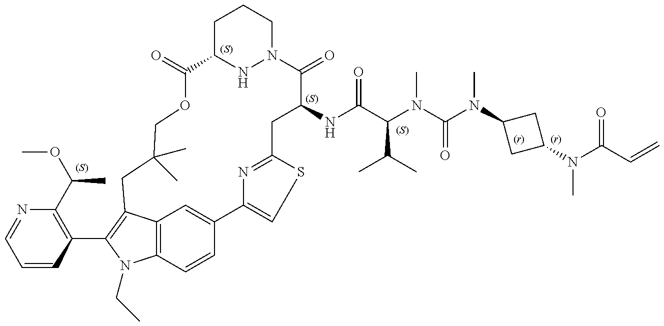 |
| BA 713 | 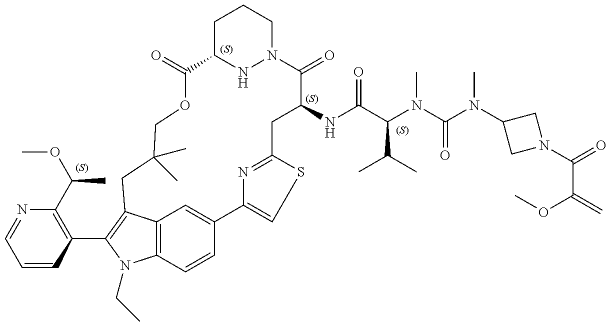 |
| BA 714 | 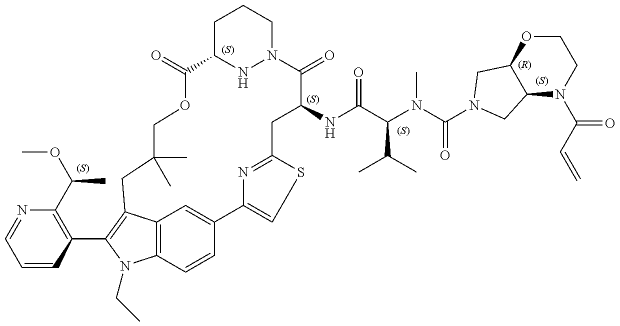 |
| BA 715 | 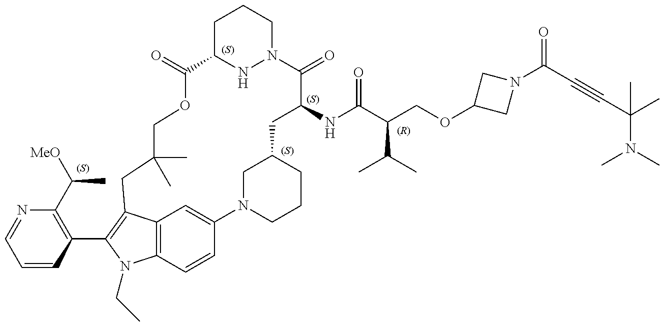 |
| BA 716 | 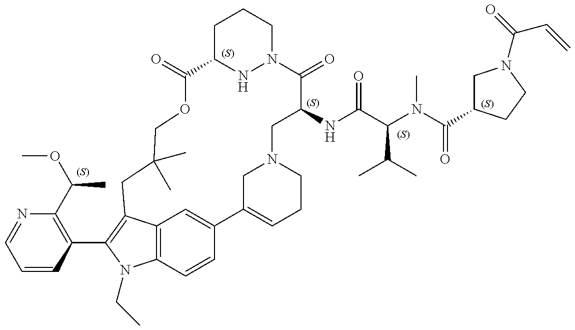 |
| BA 717 | 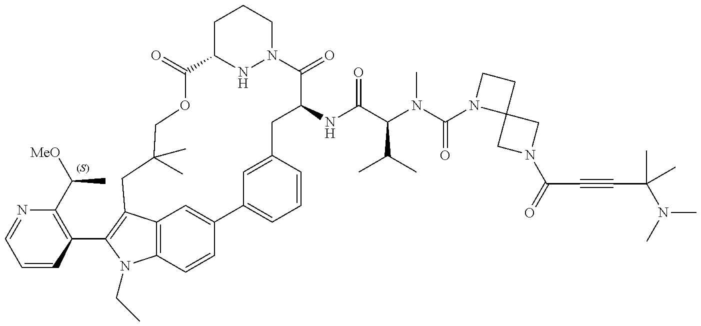 |
| BA 718 | 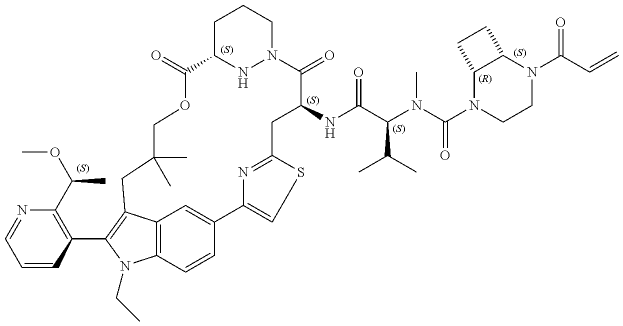 |
| BA 719 | 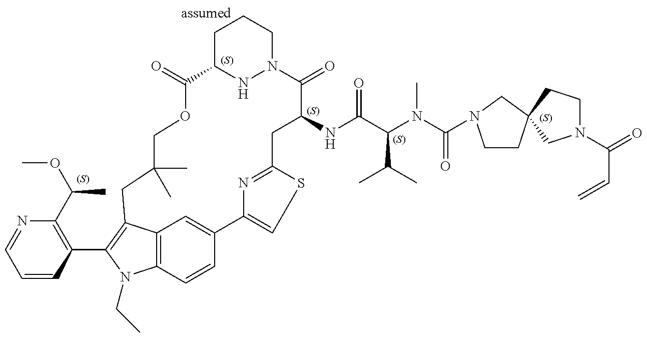 |
| BA 720 | 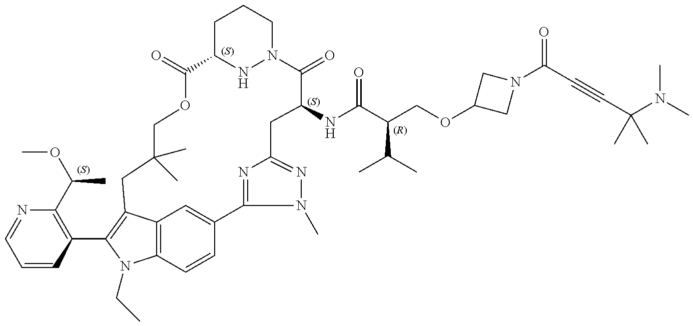 |
| BA 721 | 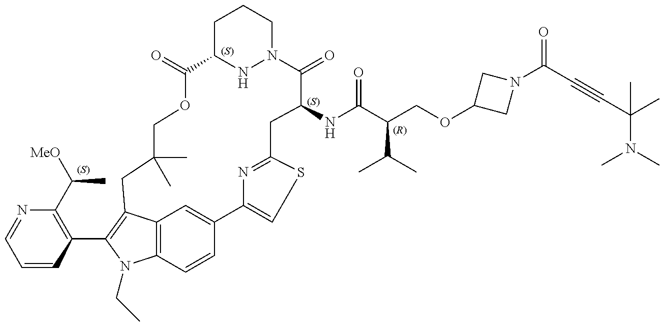 |
| BA 722 | 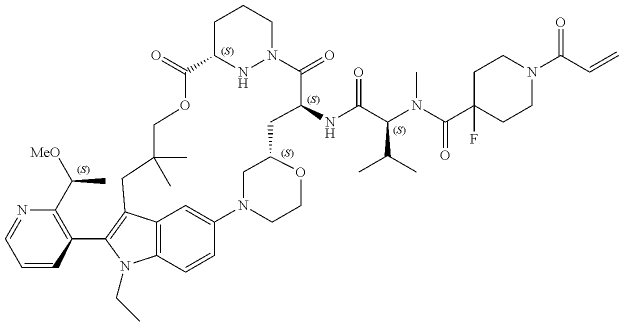 |
| BA 723 | 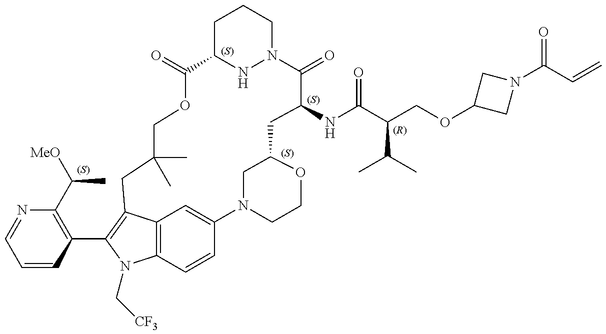 |
| BA 724 | 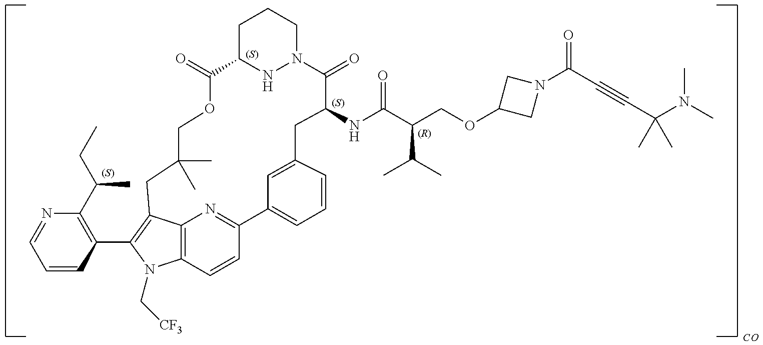 |
| BA 725 | 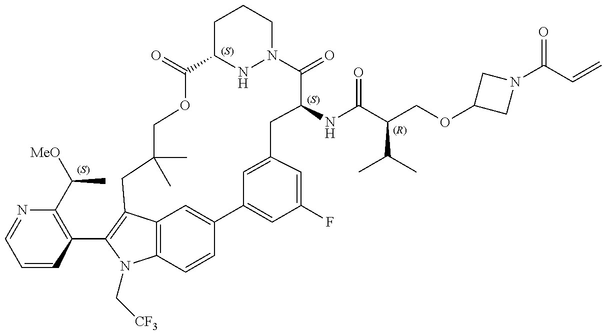 |
| BA 726 | 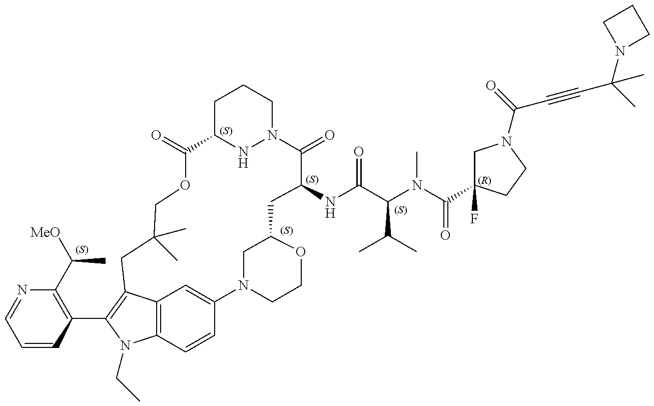 |
| BA 727 | 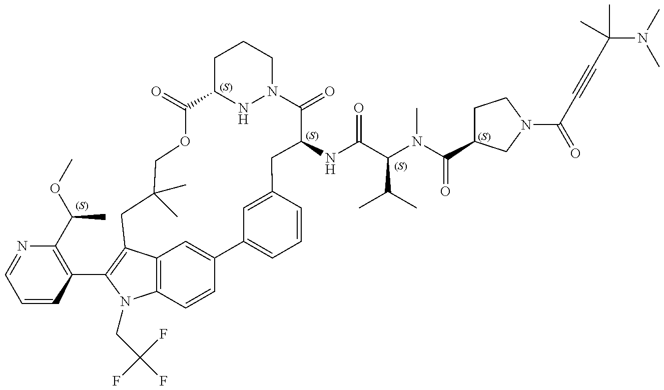 |
| BA 728 | 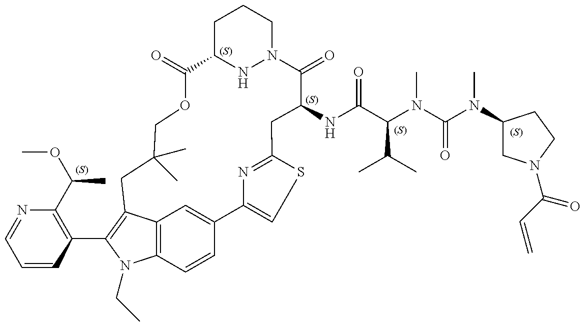 |
| BA 729 | 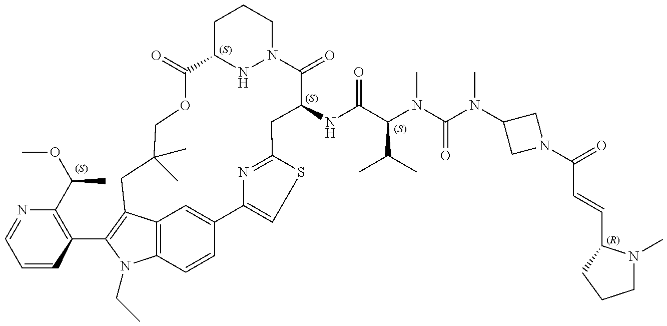 |
| BA 730 | 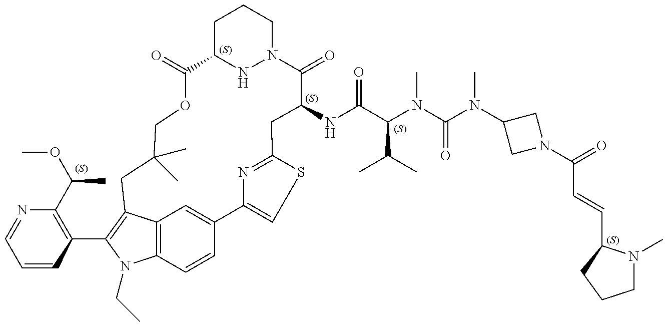 |
| BA 731 | 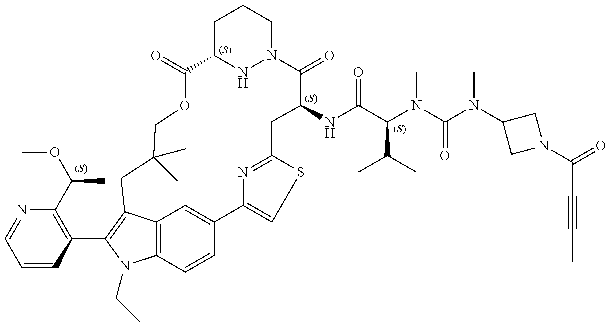 |
| BA 732 | 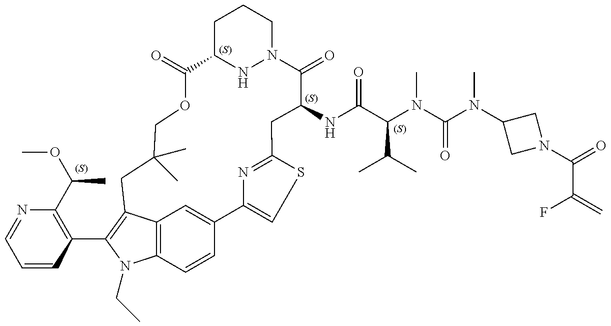 |
| BA 733 | 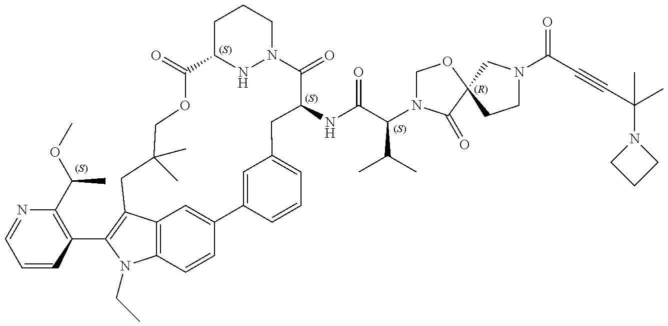 |
| BA 734 | 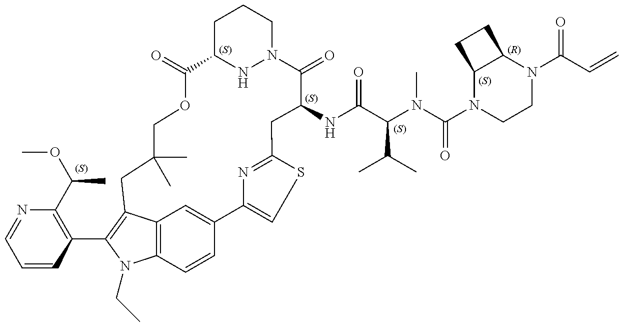 |
| BA 735 | 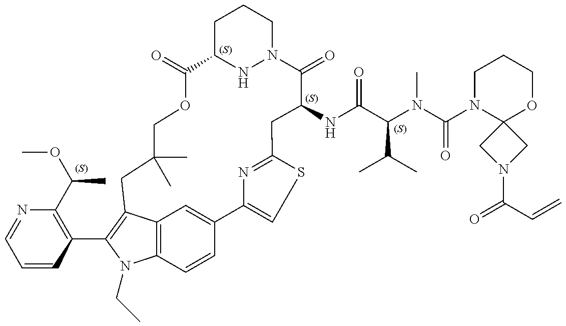 |
| BA 736 | 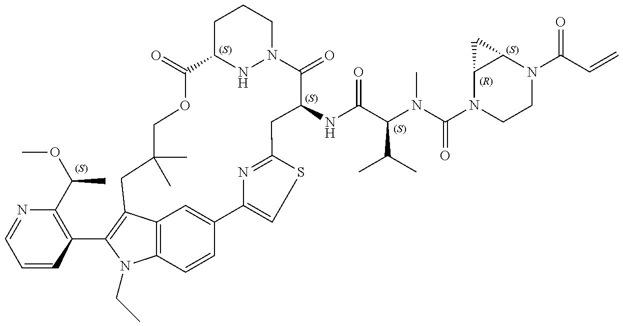 |
| BA 737 | 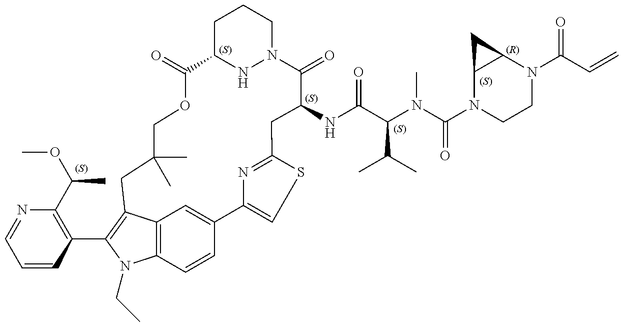 |
| BA 738 | 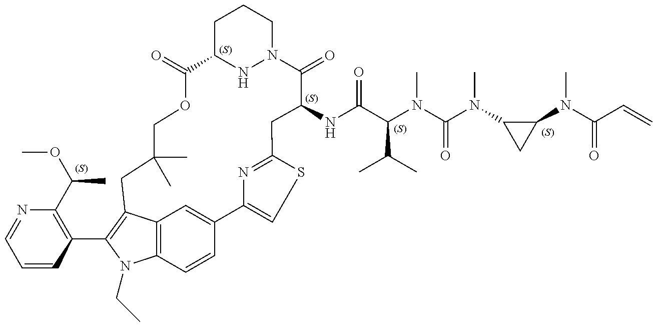 |
| BA 739 | 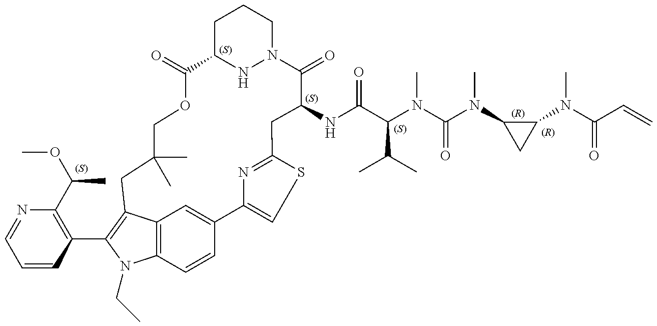 |
| BA 740 | 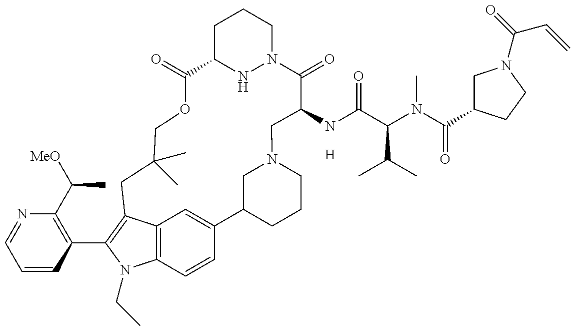 |
| BA 741 | 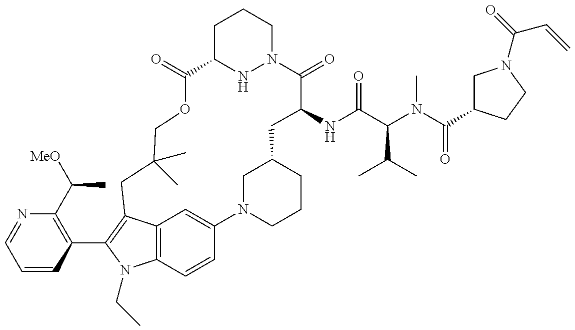 |

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated.
Brackets are to be ignored.
*The activity of this stereoisomer may, in fact, be attributable to the presence of a small amount of the stereoisomer with the (S) configuration at the —NC(O)—CH(CH₃)₂—N(CH₃)— position.

In some embodiments, a compound of Table B2 is provided, or a pharmaceutically acceptable salt thereof. In some embodiments, the RAS(ON) inhibitor is selected from Table B2, or a pharmaceutically acceptable salt or atropisomer thereof.

TABLE B2
Certain Compounds of the Present Invention

| Ex# | Structure |
|---|---|
| BB1 | 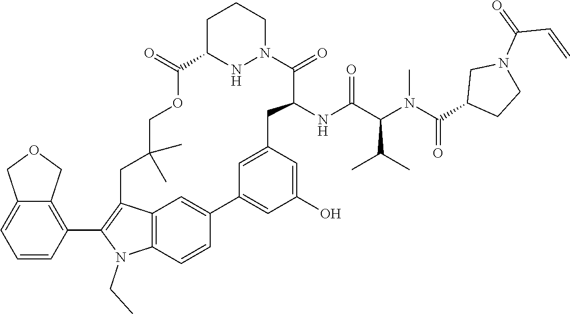 [SMILES: C=CC(=O)N1CC[C@H](C(=O)N(C)[C@H](C(=O)N[C@H]2Cc3cc(O)cc(c3)-c3ccc4c(c3)c(c(-c3cccc5c3COC5)n4CC)CC(C)(C)COC(=O)[C@@H]3CCCN(N3)C2=O)C(C)C)C1] |
| BB2 | 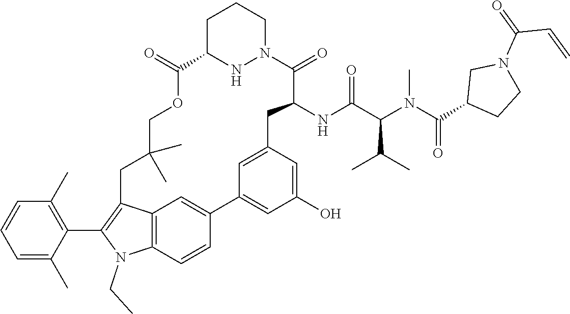 [SMILES: C=CC(=O)N1CC[C@H](C(=O)N(C)[C@H](C(=O)N[C@H]2Cc3cc(O)cc(c3)-c3ccc4c(c3)c(c(-c3c(C)cccc3C)n4CC)CC(C)(C)COC(=O)[C@@H]3CCCN(N3)C2=O)C(C)C)C1] |
| BB3 | 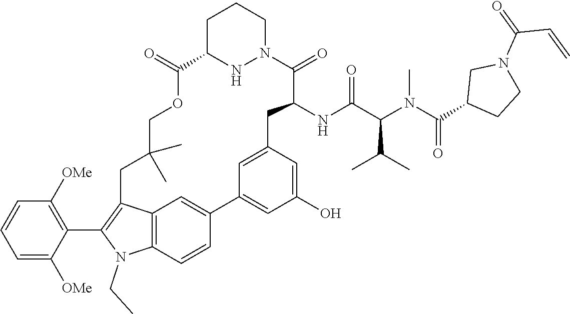 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cccc3OC)n4CC)CC(C)(C)COC(=O)[C@@H]3CCCN(N3)C2=O)C(C)C)C1] |
| BB4 | 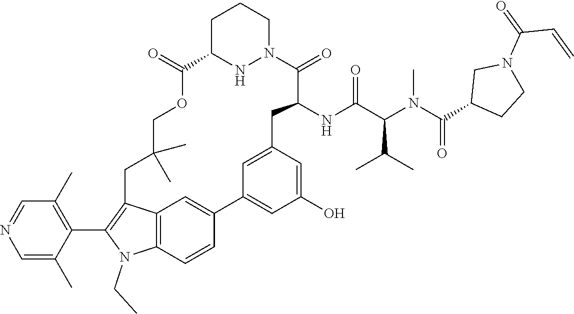 [SMILES: C=CC(=O)N1CC[C@H](C(=O)N(C)[C@H](C(=O)N[C@H]2Cc3cc(O)cc(c3)-c3ccc4c(c3)c(c(-c3c(C)cncc3C)n4CC)CC(C)(C)COC(=O)[C@@H]3CCCN(N3)C2=O)C(C)C)C1] |
| BB5 | 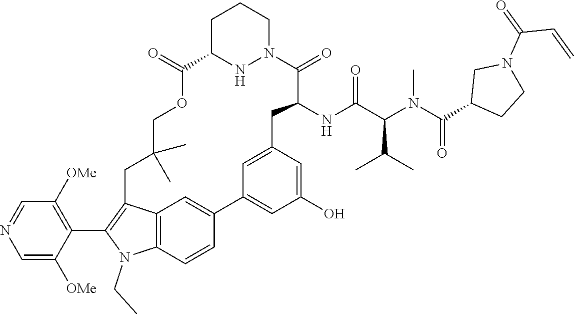 [SMILES: C=CC(=O)N1CC[C@H](C(=O)N(C)[C@H](C(=O)N[C@H]2Cc3cc(O)cc(c3)-c3ccc4c(c3)c(c(-c3c(OC)cncc3OC)n4CC)CC(C)(C)COC(=O)[C@@H]3CCCN(N3)C2=O)C(C)C)C1] |
| BB6 | 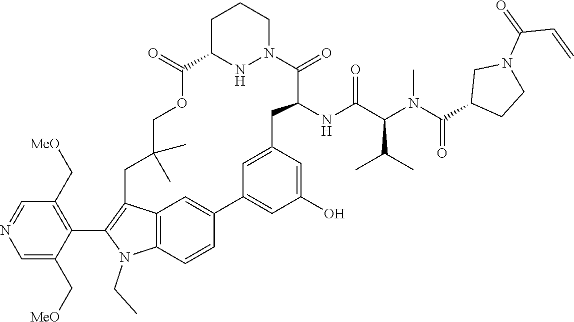 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cncc3COC)n4CC)CC(C)(C)COC(=O)[C@@H]3CCCN(N3)C2=O)C(C)C)C1] |
| BB7 | 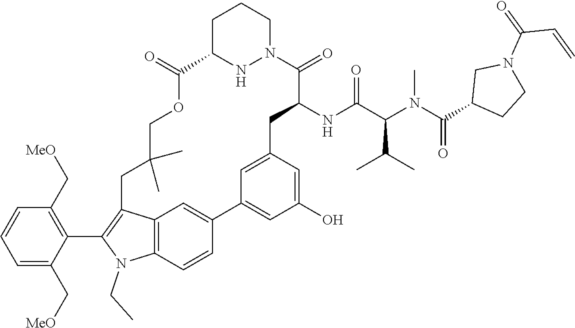 [SMILES: C=CC(=O)N1CC[C@H](C(=O)N(C)[C@H](C(=O)N[C@H]2Cc3cc(O)cc(c3)-c3ccc4c(c3)c(c(-c3c(COC)cccc3COC)n4CC)CC(C)(C)COC(=O)[C@@H]3CCCN(N3)C2=O)C(C)C)C1] |
| BB11 | 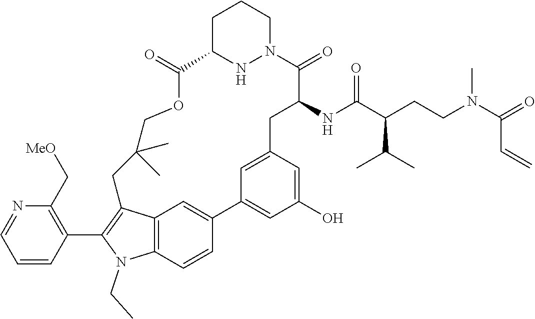 [SMILES: C=CC(=O)N(C)CC[C@H](C(=O)N[C@H]1Cc2cc(O)cc(c2)-c2ccc3c(c2)c(c(-c2cccnc2COC)n3CC)CC(C)(C)COC(=O)[C@@H]2CCCN(N2)C1=O)C(C)C] |
| BB12 | 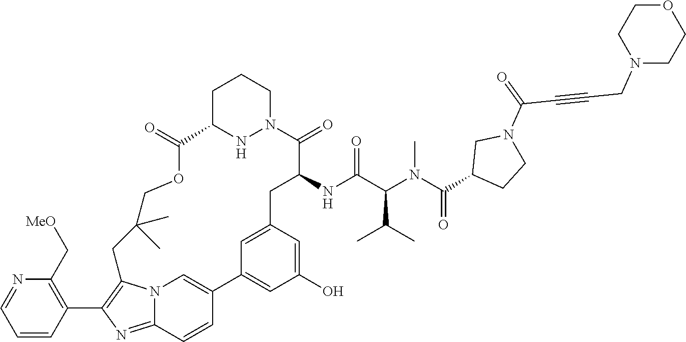 [SMILES: COCc1ncccc1-c1nc2ccc3cn2c1CC(C)(C)COC(=O)[C@@H]1CCCN(N1)C(=O)[C@@H](NC(=O)[C@H](C(C)C)N(C)C(=O)[C@H]1CCN(C(=O)C#CCN2CCOCC2)C1)Cc1cc(O)cc-3c1] |
| BB13 | 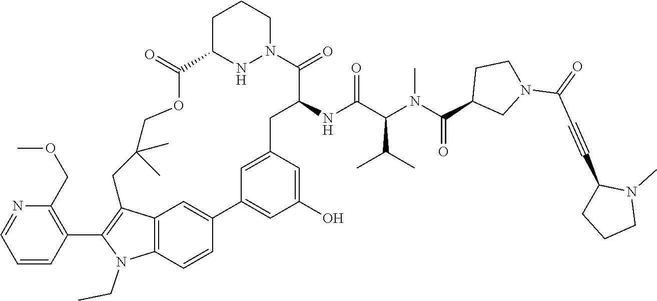 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H]3CCCN3C)C1)C(=O)N1CCC[C@H](N1)C(=O)OCC(C)(C)C2] |
| BB18 | 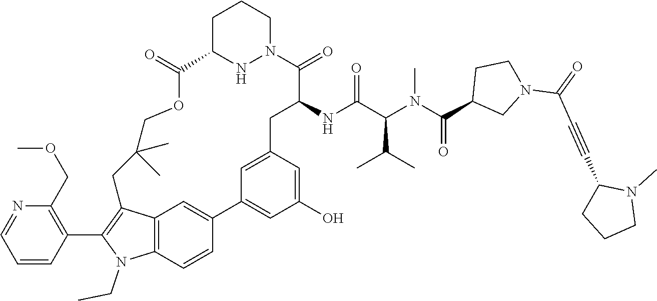 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H]3CCCN3C)C1)C(=O)N1CCC[C@H](N1)C(=O)OCC(C)(C)C2] |
| BB21 | 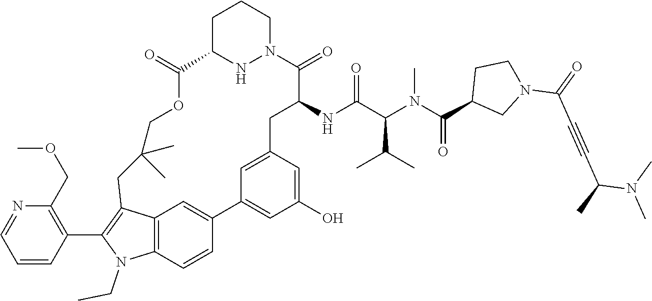 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@H](C)N(C)C)C1)C(=O)N1CCC[C@H](N1)C(=O)OCC(C)(C)C2] |
| BB22 | 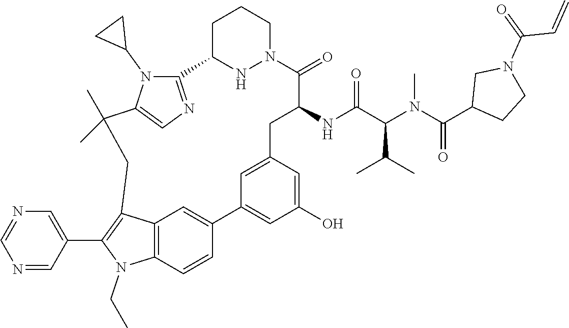 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)c3cnc(n3C3CC3)[C@@H]3CCCN(N3)C2=O)C(C)C)C1] |
| BB25 | 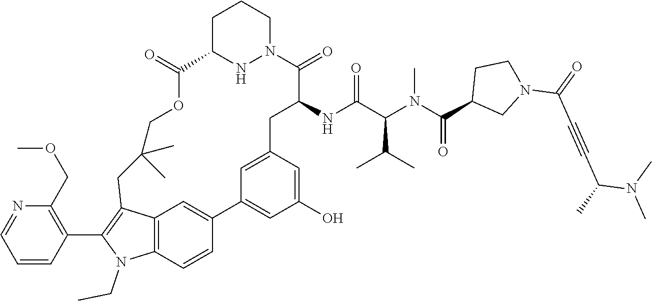 [SMILES: CCn1c(-c2cccnc2COC)c2c3cc(ccc31)-c1cc(O)cc(c1)C[C@H](NC(=O)[C@H](C(C)C)N(C)C(=O)[C@H]1CCN(C(=O)C#C[C@@H](C)N(C)C)C1)C(=O)N1CCC[C@H](N1)C(=O)OCC(C)(C)C2] |
| BB27 | 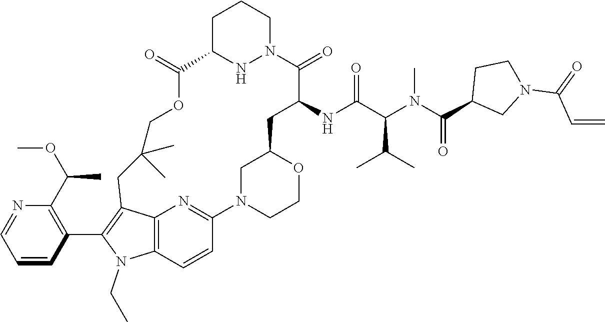 |
| BB28 | 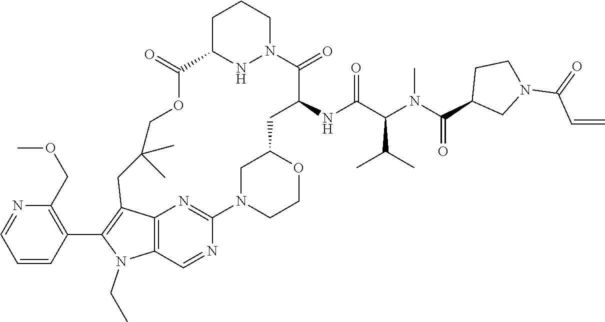 [SMILES: C=CC(=O)N1CC[C@H](C(=O)N(C)[C@H](C(=O)N[C@H]2C[C@H]3CN(CCO3)c3ncc4c(n3)c(c(-c3cccnc3COC)n4CC)CC(C)(C)COC(=O)[C@@H]3CCCN(N3)C2=O)C(C)C)C1] |
| BB29 | 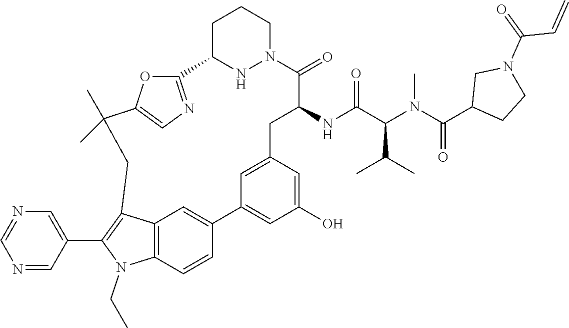 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC(C)(C)C3=CN=C(O3)[C@@H]3CCCN(N3)C2=O)C(C)C)C1] |
| BB30 | 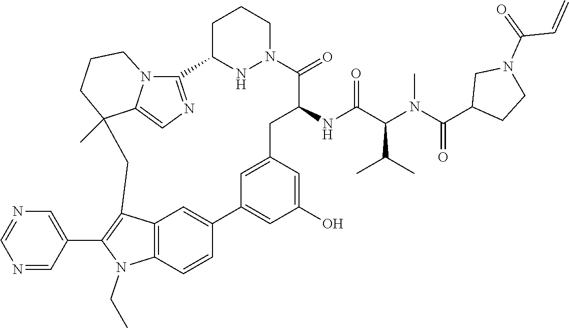 [SMILES: C=CC(=O)N1CCC(C(=O)N(C)[C@H](C(=O)N[C@H]2Cc3cc(O)cc(c3)-c3ccc4c(c3)c(c(-c3cncnc3)n4CC)CC3(C)CCCn4c3cnc4[C@@H]3CCCN(N3)C2=O)C(C)C)C1] |
| BB32 | 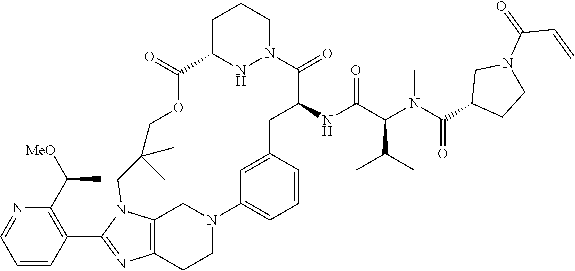 [SMILES: C=CC(=O)N1CC[C@H](C(=O)N(C)[C@H](C(=O)N[C@H]2Cc3cccc(c3)N3CCc4nc(-c5cccnc5[C@H](C)OC)n(c4C3)CC(C)(C)COC(=O)[C@@H]3CCCN(N3)C2=O)C(C)C)C1] |
| BB34 | 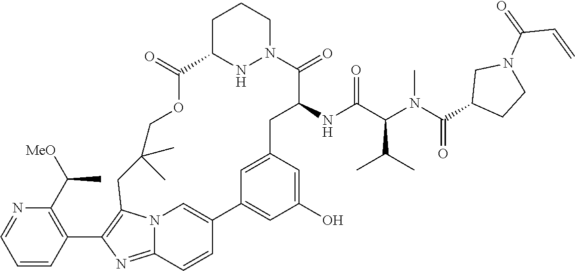 [SMILES: C=CC(=O)N1CC[C@H](C(=O)N(C)[C@H](C(=O)N[C@H]2Cc3cc(O)cc(c3)-c3ccc4nc(-c5cccnc5[C@H](C)OC)c(n4c3)CC(C)(C)COC(=O)[C@@H]3CCCN(N3)C2=O)C(C)C)C1] |
| BB38 | 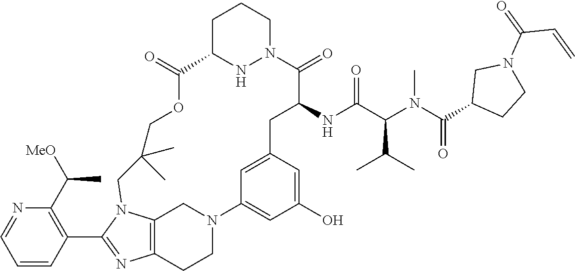 [SMILES: C=CC(=O)N1CC[C@H](C(=O)N(C)[C@H](C(=O)N[C@H]2Cc3cc(O)cc(c3)N3CCc4nc(-c5cccnc5[C@H](C)OC)n(c4C3)CC(C)(C)COC(=O)[C@@H]3CCCN(N3)C2=O)C(C)C)C1] |
| BB47 | 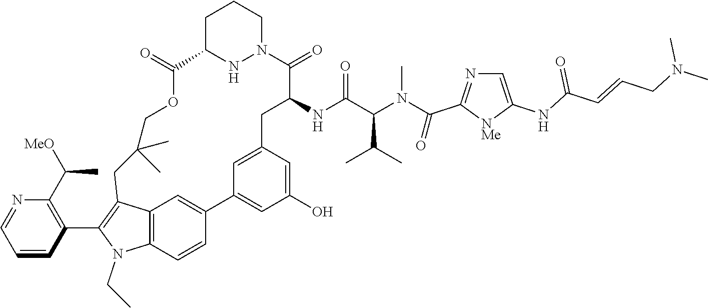 |
| BB64 | 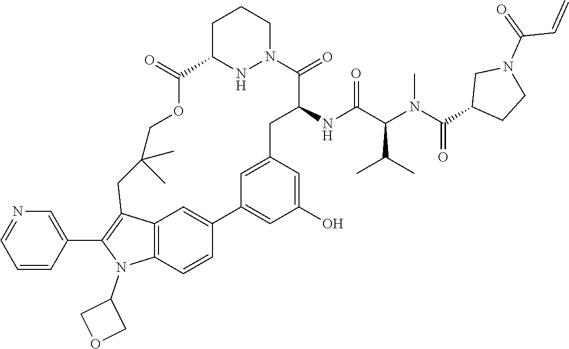 [SMILES: C=CC(=O)N1CC[C@H](C(=O)N(C)[C@H](C(=O)N[C@H]2Cc3cc(O)cc(c3)-c3ccc4c(c3)c(c(-c3cccnc3)n4C3COC3)CC(C)(C)COC(=O)[C@@H]3CCCN(N3)C2=O)C(C)C)C1] |
| BB65 | 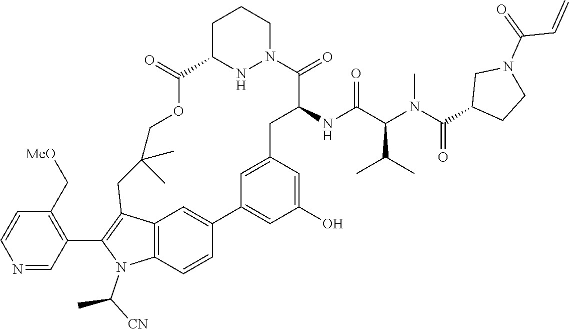 [SMILES: C=CC(=O)N1CC[C@H](C(=O)N(C)[C@H](C(=O)N[C@H]2Cc3cc(O)cc(c3)-c3ccc4c(c3)c(c(-c3cnccc3COC)n4[C@H](C)C#N)CC(C)(C)COC(=O)[C@@H]3CCCN(N3)C2=O)C(C)C)C1] |
| BB66 | 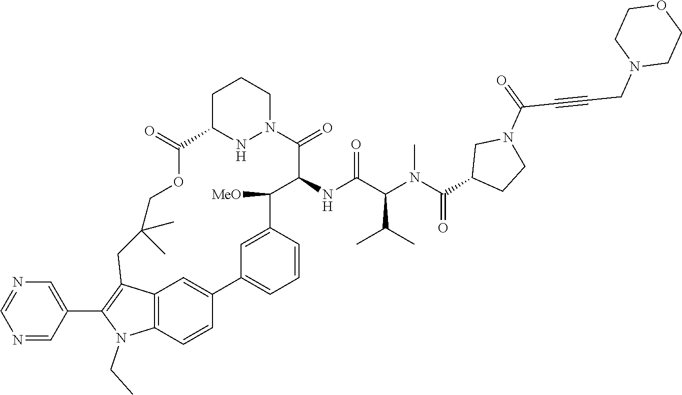 [SMILES: CCn1c(-c2cncnc2)c2c3cc(ccc31)-c1cccc(c1)[C@@H](OC)[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB70 | 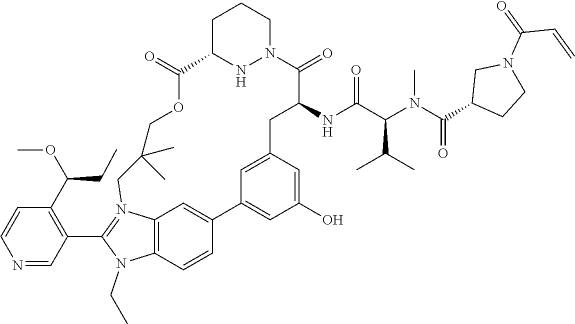 |
| BB73 | 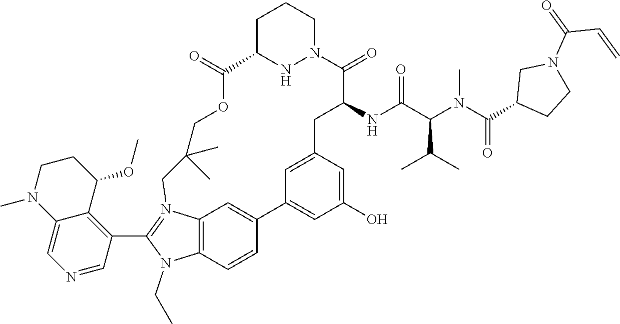 |
| BB74 | 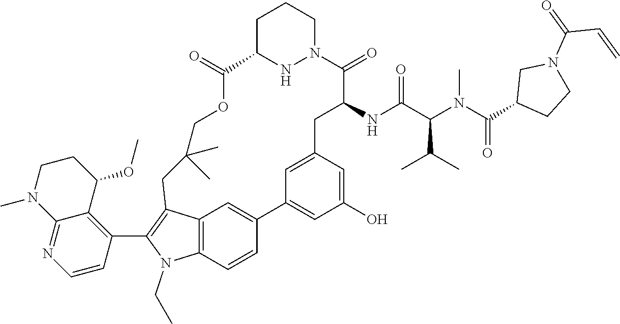 [SMILES: C=CC(=O)N1CC[C@H](C(=O)N(C)[C@H](C(=O)N[C@H]2Cc3cc(O)cc(c3)-c3ccc4c(c3)c(c(-c3ccnc5c3[C@@H](OC)CCN5C)n4CC)CC(C)(C)COC(=O)[C@@H]3CCCN(N3)C2=O)C(C)C)C1] |
| BB75 | 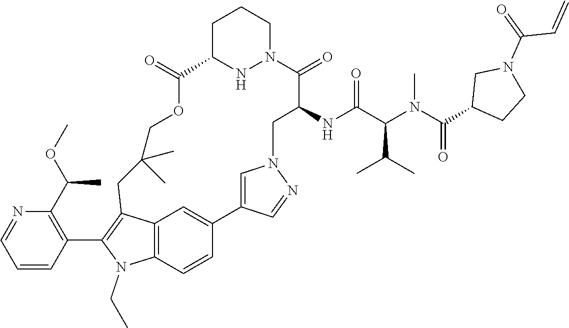 [SMILES: C=CC(=O)N1CC[C@H](C(=O)N(C)[C@H](C(=O)N[C@H]2Cn3cc(cn3)-c3ccc4c(c3)c(c(-c3cccnc3[C@H](C)OC)n4CC)CC(C)(C)COC(=O)[C@@H]3CCCN(N3)C2=O)C(C)C)C1] |
| BB76 | 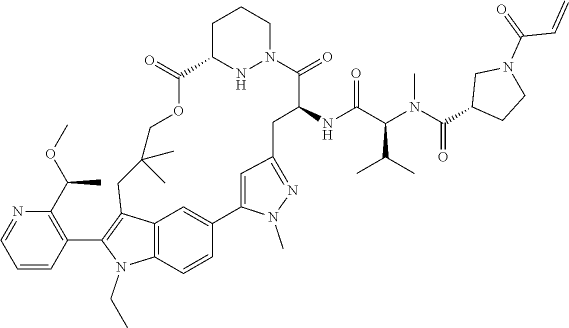 [SMILES: C=CC(=O)N1CC[C@H](C(=O)N(C)[C@H](C(=O)N[C@H]2Cc3cc(n(C)n3)-c3ccc4c(c3)c(c(-c3cccnc3[C@H](C)OC)n4CC)CC(C)(C)COC(=O)[C@@H]3CCCN(N3)C2=O)C(C)C)C1] |
| BB77 | 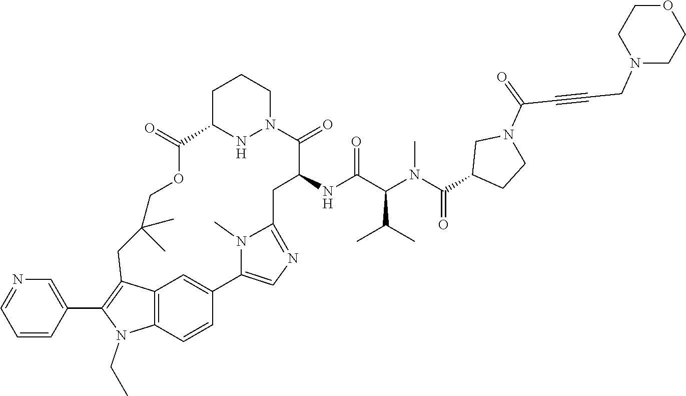 [SMILES: CCn1c(-c2cccnc2)c2c3cc(ccc31)-c1cnc(n1C)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB81 | 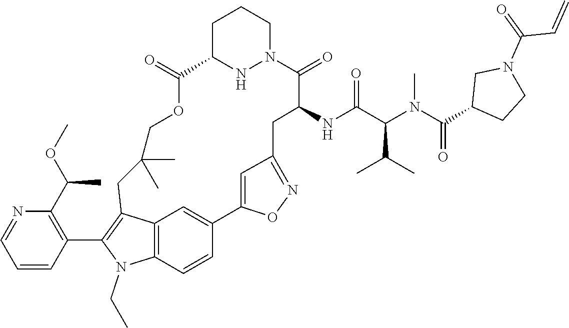 [SMILES: C=CC(=O)N1CC[C@H](C(=O)N(C)[C@H](C(=O)N[C@H]2Cc3cc(on3)-c3ccc4c(c3)c(c(-c3cccnc3[C@H](C)OC)n4CC)CC(C)(C)COC(=O)[C@@H]3CCCN(N3)C2=O)C(C)C)C1] |
| BB83 | 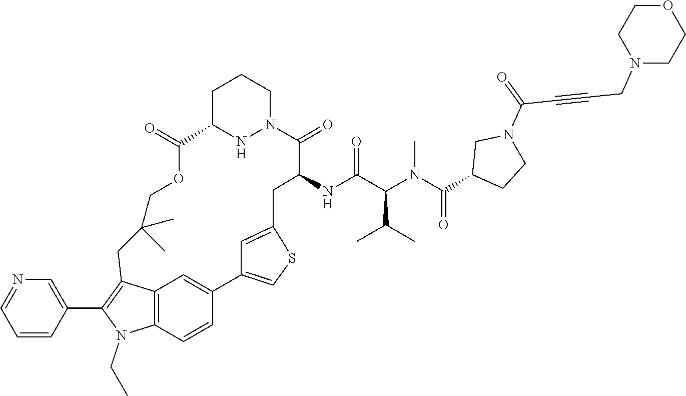 [SMILES: CCn1c(-c2cccnc2)c2c3cc(ccc31)-c1csc(c1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB85 | 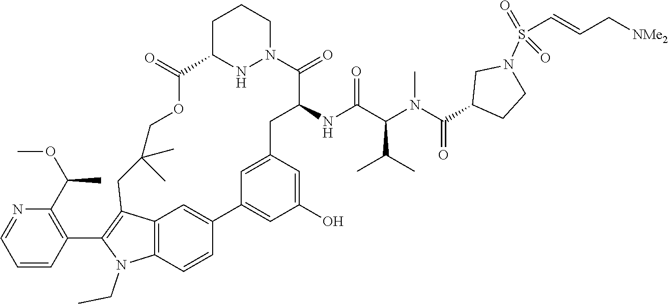 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)/C=C/CN(C)C)C1)C(=O)N1CCC[C@H](N1)C(=O)OCC(C)(C)C2] |
| BB86 | 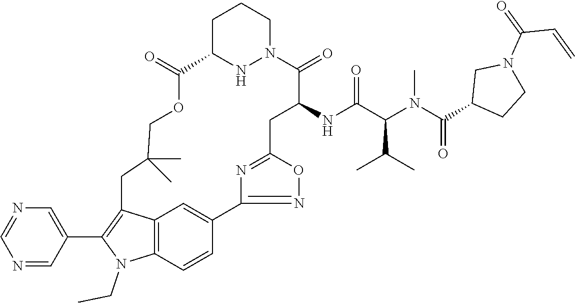 [SMILES: C=CC(=O)N1CC[C@H](C(=O)N(C)[C@H](C(=O)N[C@H]2Cc3nc(no3)-c3ccc4c(c3)c(c(-c3cncnc3)n4CC)CC(C)(C)COC(=O)[C@@H]3CCCN(N3)C2=O)C(C)C)C1] |
| BB87 | 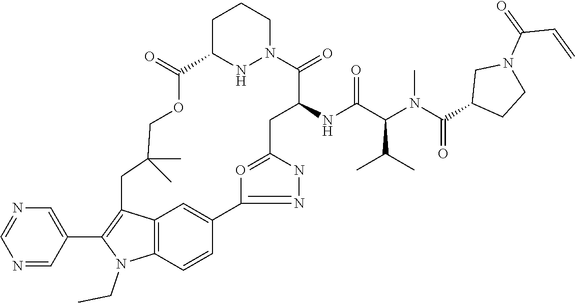 |
| BB88 | 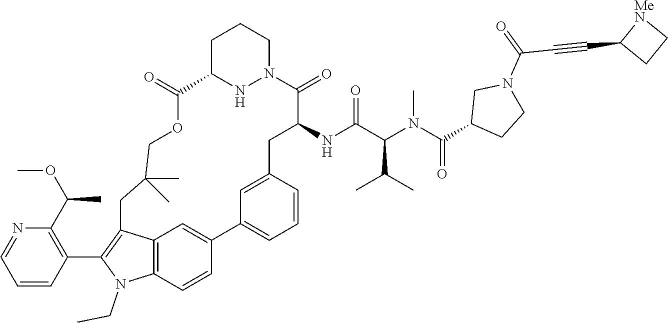 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| BB89 | 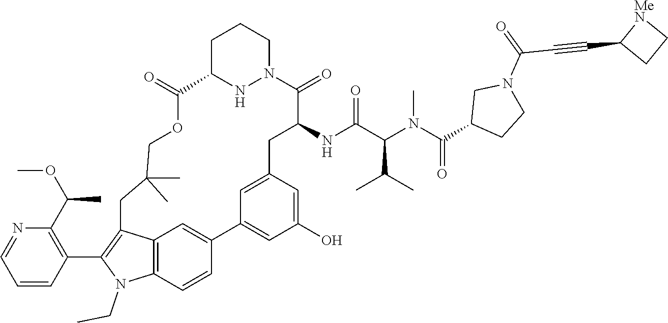 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(C(=O)C#C[C@@H]3CCN3C)C1)C(=O)N1CCC[C@H](N1)C(=O)OCC(C)(C)C2] |
| BB90 | 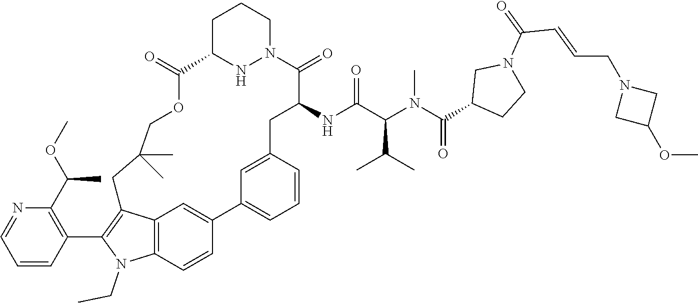 [SMILES: CCn1c(-c2cccnc2[C@H](C)OC)c2c3cc(ccc31)-c1cccc(c1)C[C@H](NC(=O)[C@H](C(C)C)N(C)C(=O)[C@H]1CCN(C(=O)/C=C/CN3CC(OC)C3)C1)C(=O)N1CCC[C@H](N1)C(=O)OCC(C)(C)C2] |
| BB91 | 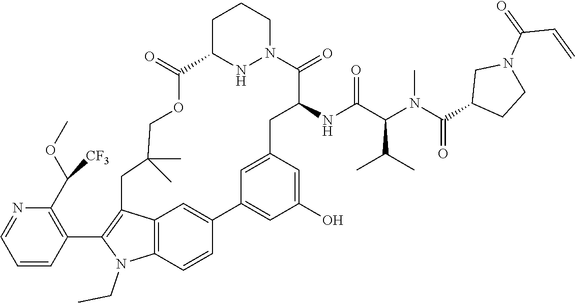 [SMILES: C=CC(=O)N1CC[C@H](C(=O)N(C)[C@H](C(=O)N[C@H]2Cc3cc(O)cc(c3)-c3ccc4c(c3)c(c(-c3cccnc3[C@@H](OC)C(F)(F)F)n4CC)CC(C)(C)COC(=O)[C@@H]3CCCN(N3)C2=O)C(C)C)C1] |
| BB96 | 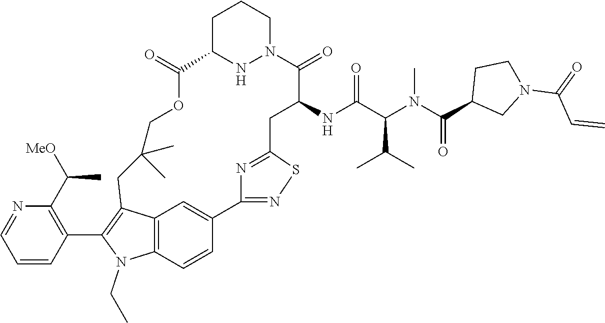 [SMILES: C=CC(=O)N1CC[C@H](C(=O)N(C)[C@H](C(=O)N[C@H]2Cc3nc(ns3)-c3ccc4c(c3)c(c(-c3cccnc3[C@H](C)OC)n4CC)CC(C)(C)COC(=O)[C@@H]3CCCN(N3)C2=O)C(C)C)C1] |
| BB97 | 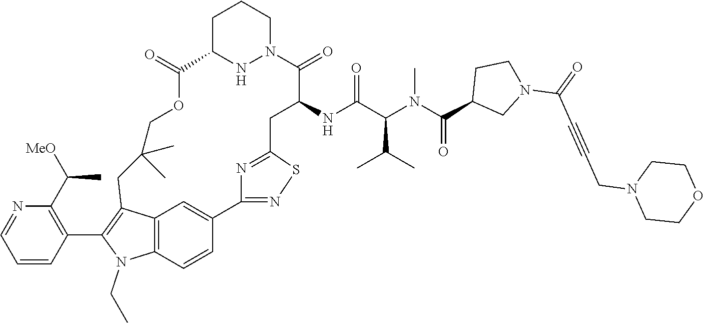 [SMILES: CCn1c(-c2cccnc2[C@H](C)OC)c2c3cc(ccc31)-c1nsc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB102 | 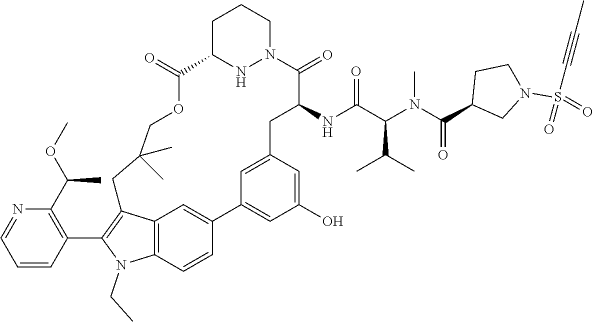 [SMILES: CC#CS(=O)(=O)N1CC[C@H](C(=O)N(C)[C@H](C(=O)N[C@H]2Cc3cc(O)cc(c3)-c3ccc4c(c3)c(c(-c3cccnc3[C@H](C)OC)n4CC)CC(C)(C)COC(=O)[C@@H]3CCCN(N3)C2=O)C(C)C)C1] |
| BB103 | 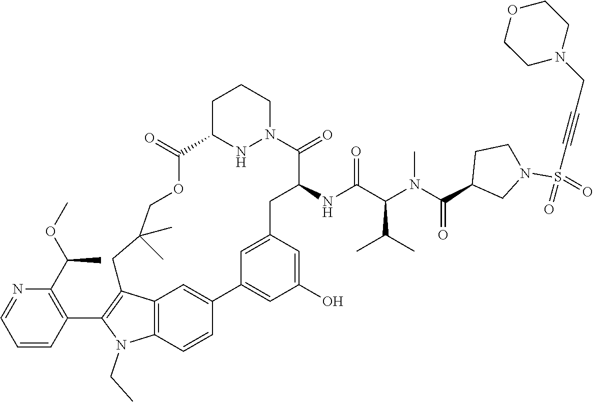 [SMILES: CCn1c(-c2cccnc2[C@H](C)OC)c2c3cc(ccc31)-c1cc(O)cc(c1)C[C@H](NC(=O)[C@H](C(C)C)N(C)C(=O)[C@H]1CCN(S(=O)(=O)C#CCN3CCOCC3)C1)C(=O)N1CCC[C@H](N1)C(=O)OCC(C)(C)C2] |
| BB104 | 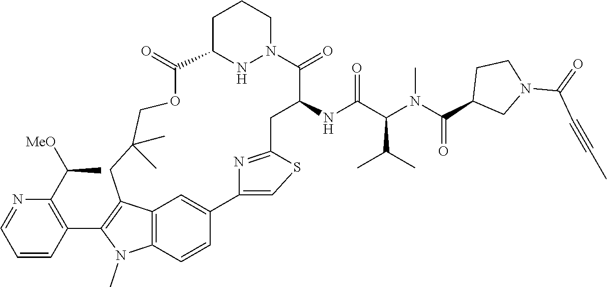 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C)CC(C)(C)COC(=O)[C@@H]3CCCN(N3)C2=O)C(C)C)C1] |
| BB106 | 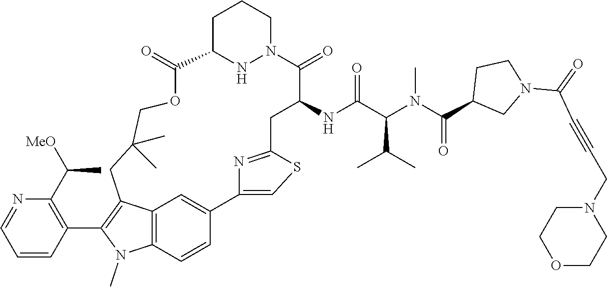 [SMILES: CO[C@@H](C)c1ncccc1-c1c2c3cc(ccc3n1C)-c1csc(n1)C[C@H](NC(=O)[C@H](C(C)C)N(C)C(=O)[C@H]1CCN(C(=O)C#CCN3CCOCC3)C1)C(=O)N1CCC[C@H](N1)C(=O)OCC(C)(C)C2] |
| BB107 | 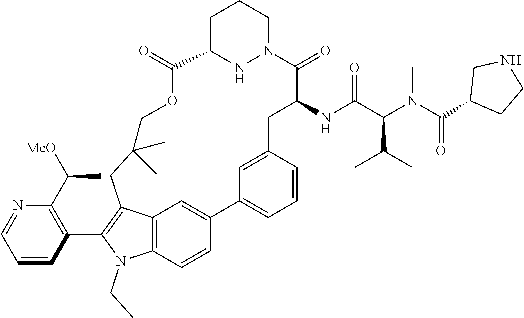 |
| BB109 | 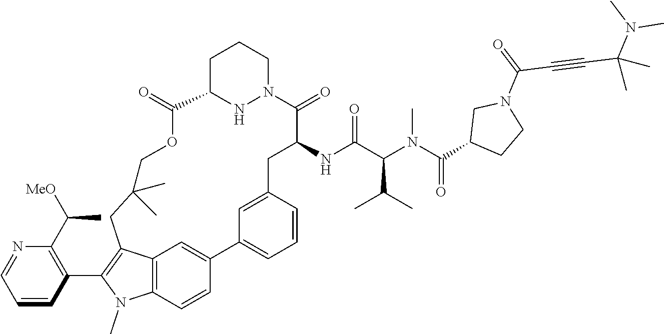 |
| BB111 | 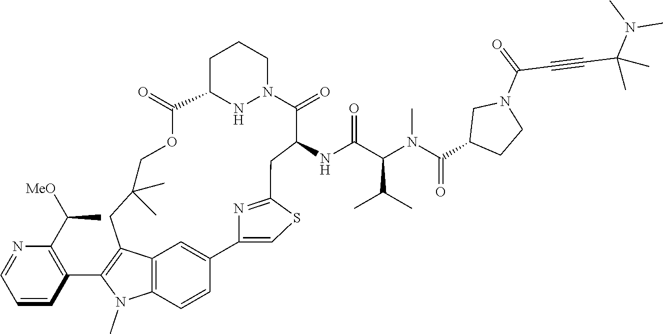 |
| BB112 | 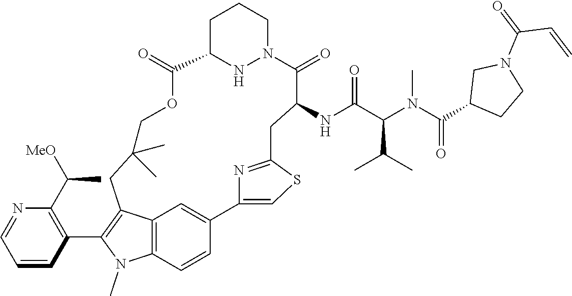 |
| BB113 | 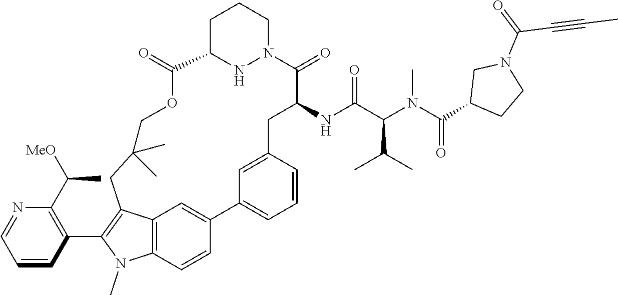 |
| BB115 | 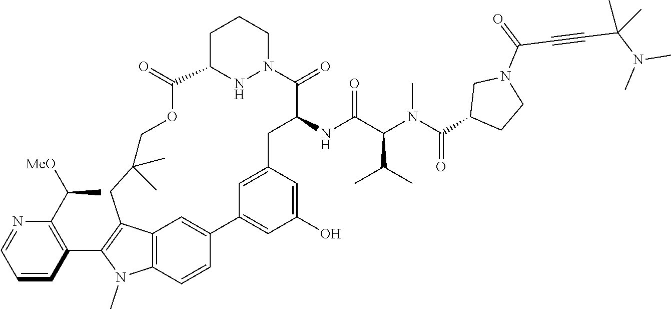 |
| BB116 | 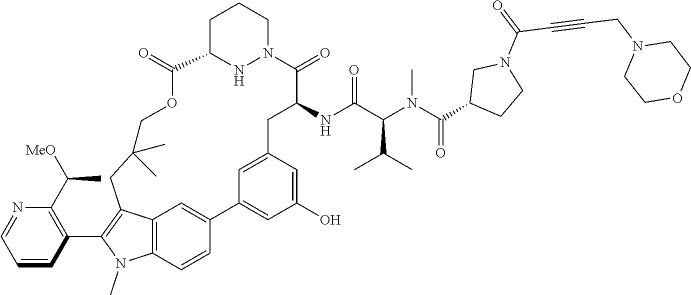 |
| BB117 | 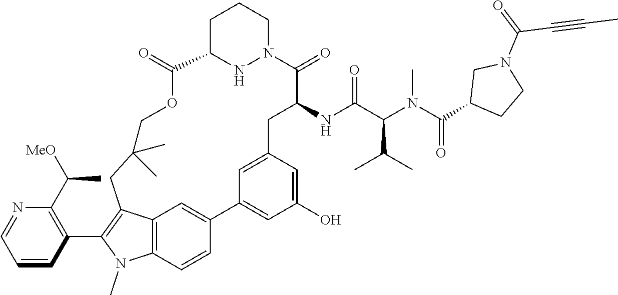 |
| BB118 | 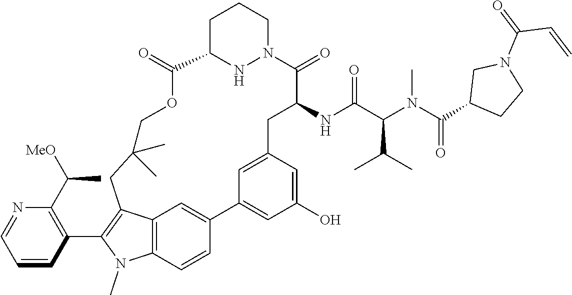 |
| BB119 | 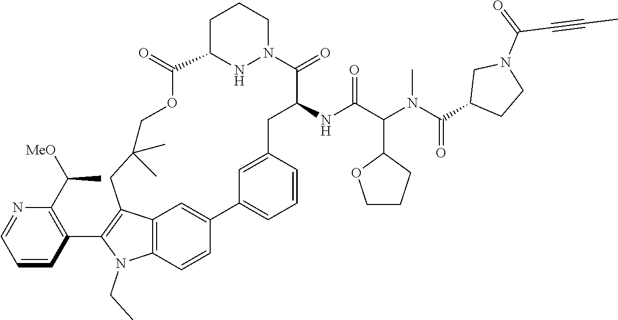 |
| BB120 | 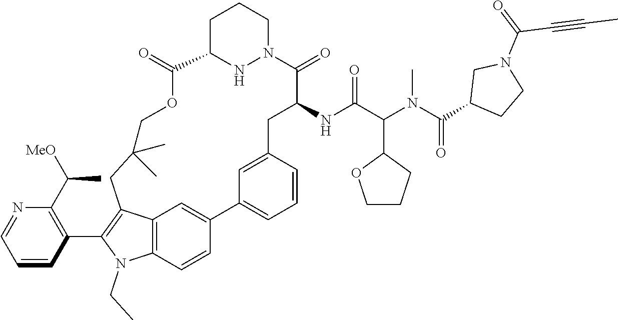 |
| BB121 | 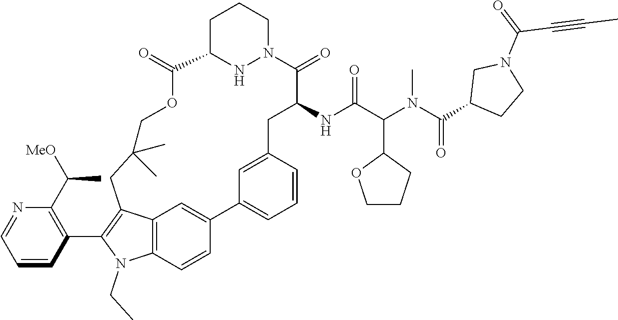 |
| BB122 | 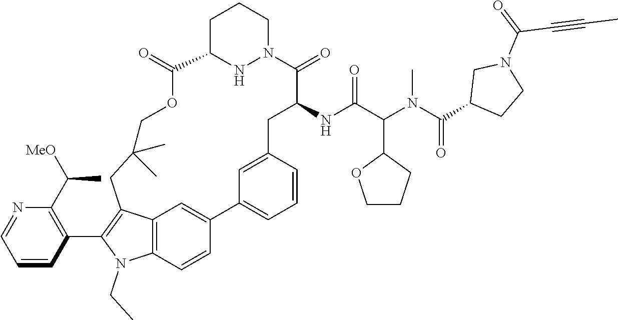 |
| BB123 | 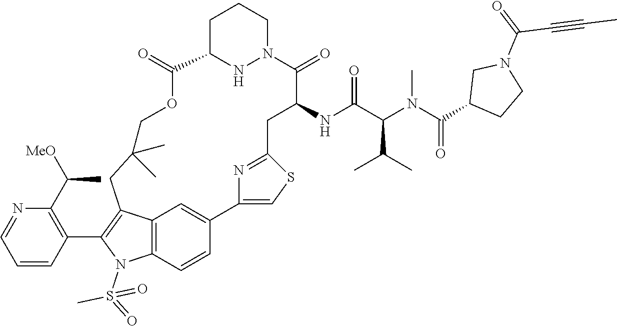 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4S(C)(=O)=O)CC(C)(C)COC(=O)[C@@H]3CCCN(N3)C2=O)C(C)C)C1] |
| BB124 | 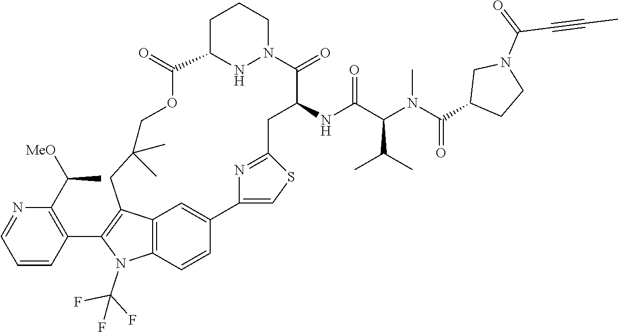 [SMILES: CC#CC(=O)N1CC[C@H](C(=O)N(C)[C@H](C(=O)N[C@H]2Cc3nc(cs3)-c3ccc4c(c3)c(c(-c3cccnc3[C@H](C)OC)n4C(F)(F)F)CC(C)(C)COC(=O)[C@@H]3CCCN(N3)C2=O)C(C)C)C1] |
| BB126 | 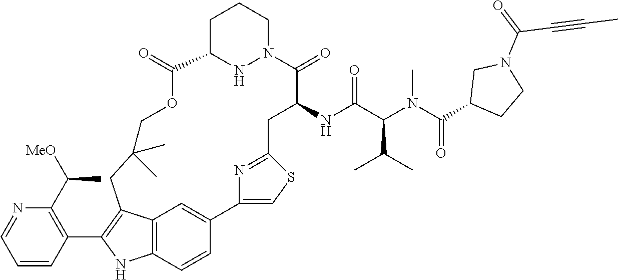 [SMILES: CC#CC(=O)N1CC[C@H](C(=O)N(C)[C@H](C(=O)N[C@H]2Cc3nc(cs3)-c3ccc4[nH]c(-c5cccnc5[C@H](C)OC)c(c4c3)CC(C)(C)COC(=O)[C@@H]3CCCN(N3)C2=O)C(C)C)C1] |
| BB127 | 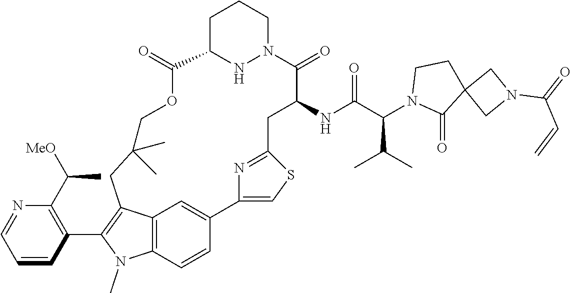 |
| BB128 | 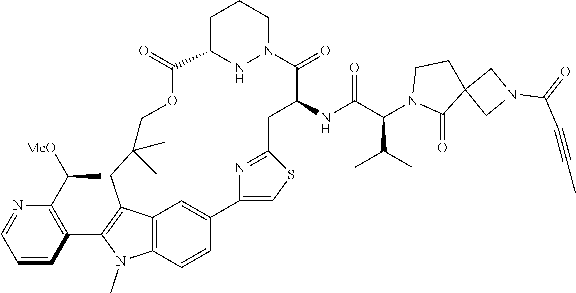 |
| BB129 | 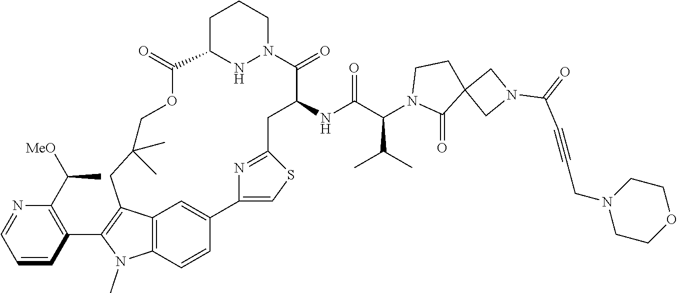 |
| BB130 | 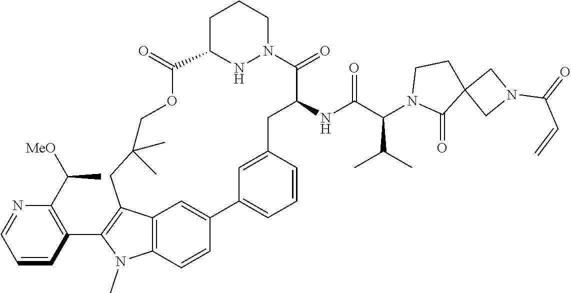 |
| BB131 | 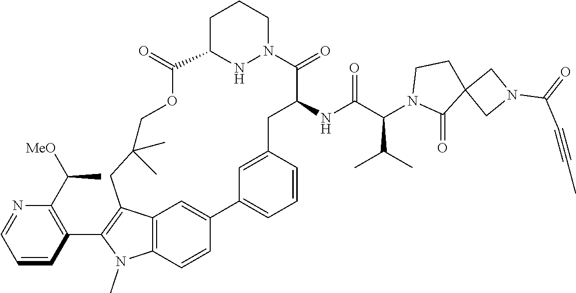 |
| BB132 | 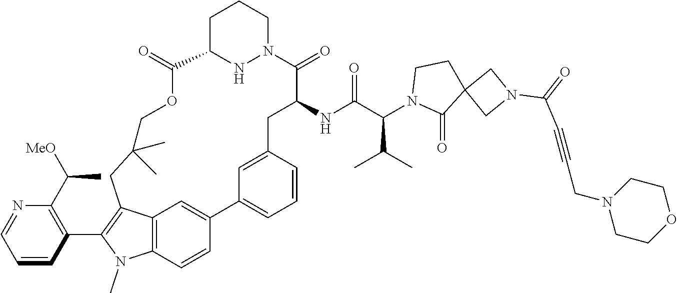 |
| BB139 | 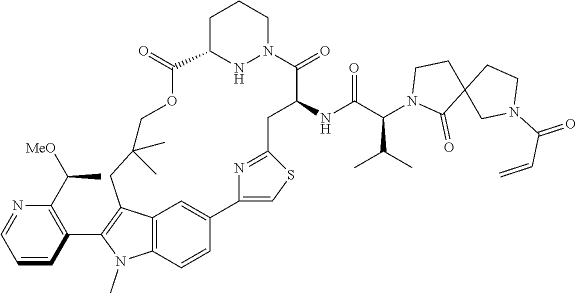 |
| BB140 | 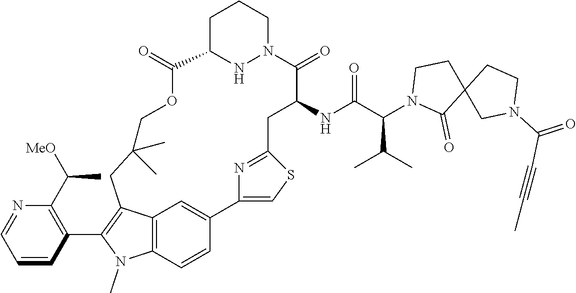 |
| BB141 | 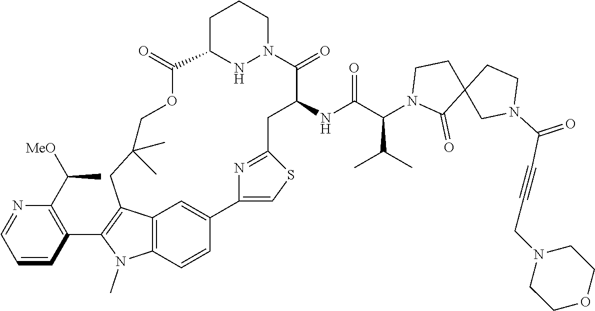 |
| BB142 | 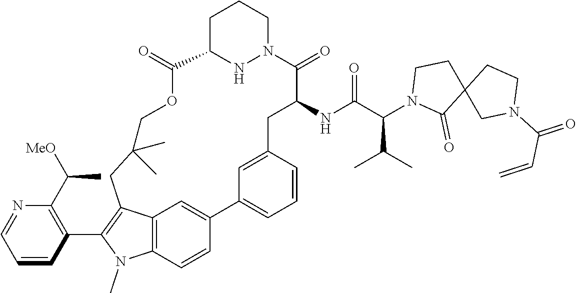 |
| BB143 | 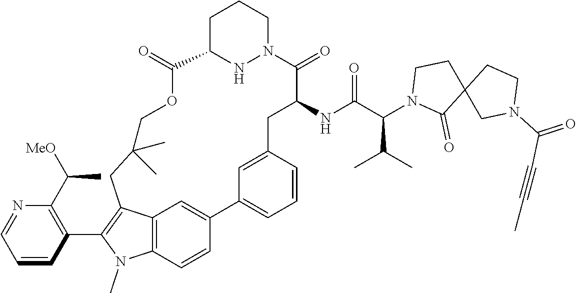 |
| BB144 | 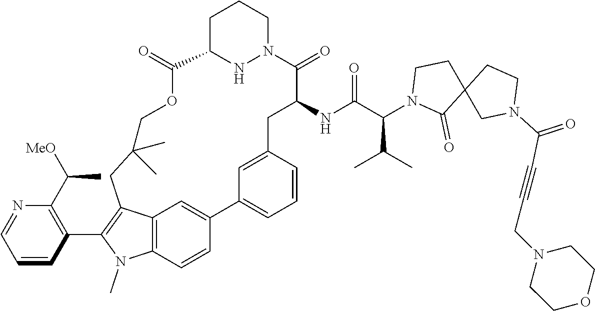 |
| BB145 | 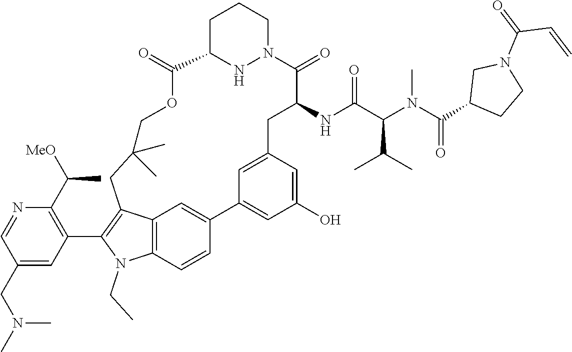 [SMILES: C=CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB146 | 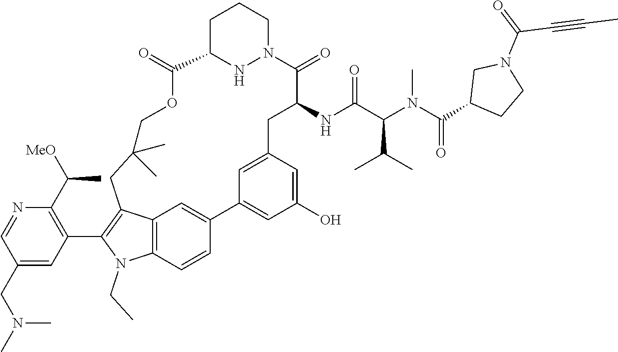 [SMILES: CC#CC(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB147 | 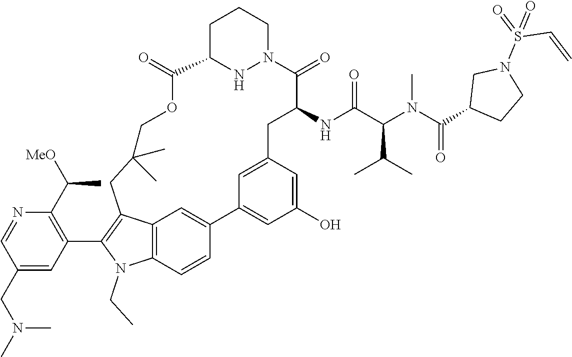 [SMILES: C=CS(=O)(=O)N1CC[C@H](C(=O)N(C)[C@H](C(=O)N[C@H]2Cc3cc(O)cc(c3)-c3ccc4c(c3)c(c(-c3cc(CN(C)C)cnc3[C@H](C)OC)n4CC)CC(C)(C)COC(=O)[C@@H]3CCCN(N3)C2=O)C(C)C)C1] |
| BB148 | 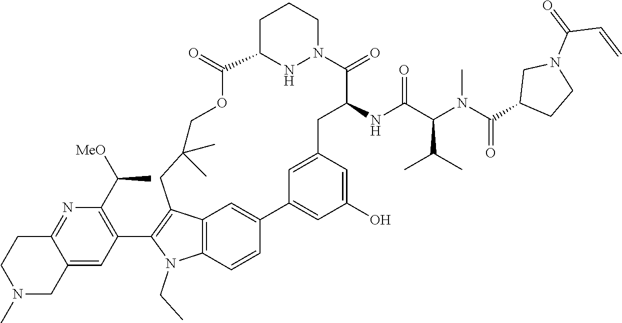 [SMILES: C=CC(=O)N1CC[C@H](C(=O)N(C)[C@H](C(=O)N[C@H]2Cc3cc(O)cc(c3)-c3ccc4c(c3)c(c(-c3cc5c(nc3[C@H](C)OC)CCN(C)C5)n4CC)CC(C)(C)COC(=O)[C@@H]3CCCN(N3)C2=O)C(C)C)C1] |
| BB149 | 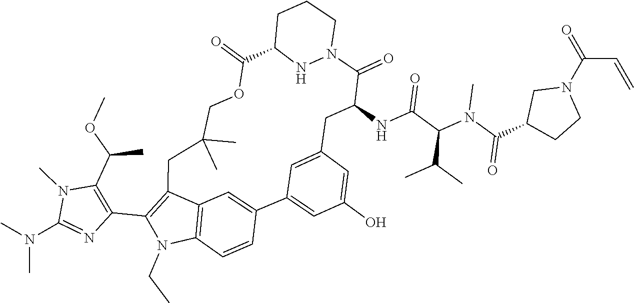 [SMILES: C=CC(=O)N1CC[C@H](C(=O)N(C)[C@H](C(=O)N[C@H]2Cc3cc(O)cc(c3)-c3ccc4c(c3)c(c(-c3nc(N(C)C)n(C)c3[C@H](C)OC)n4CC)CC(C)(C)COC(=O)[C@@H]3CCCN(N3)C2=O)C(C)C)C1] |
| BB150 | 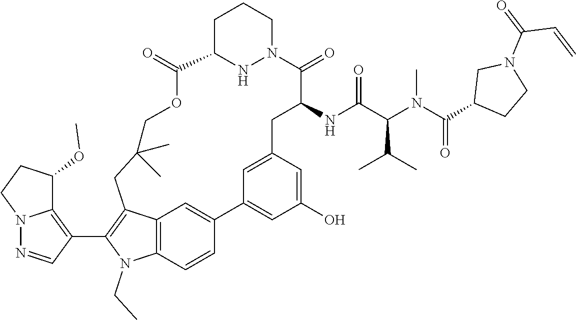 [SMILES: C=CC(=O)N1CC[C@H](C(=O)N(C)[C@H](C(=O)N[C@H]2Cc3cc(O)cc(c3)-c3ccc4c(c3)c(c(-c3cnn5c3[C@@H](OC)CC5)n4CC)CC(C)(C)COC(=O)[C@@H]3CCCN(N3)C2=O)C(C)C)C1] |
| BB161 | 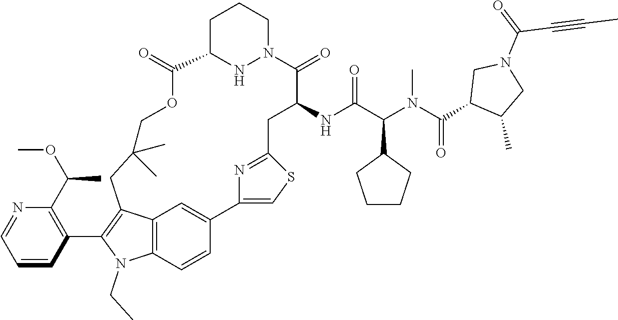 |
| BB162 | 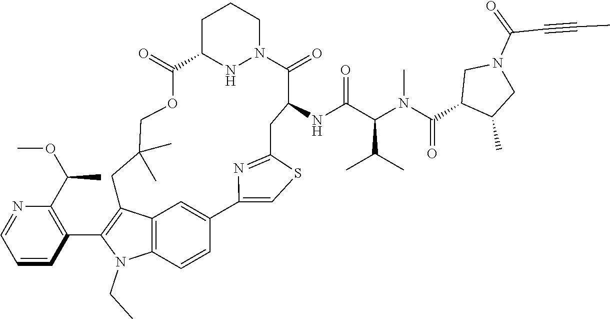 |
| BB163 | 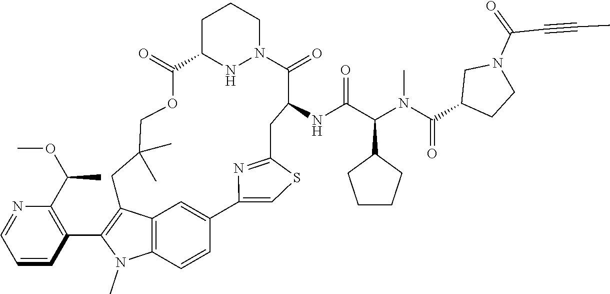 |
| BB164 | 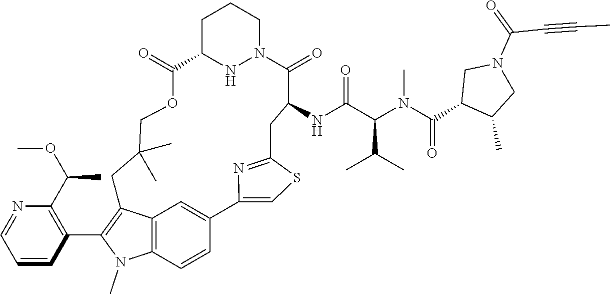 |
| BB165 | 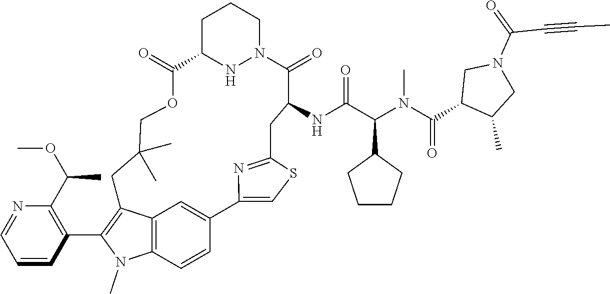 |
| BB167 | 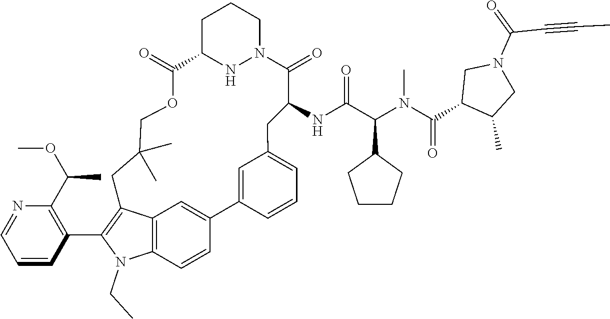 |
| BB168 | 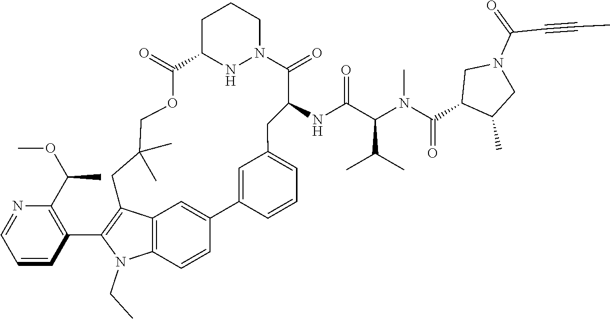 |
| BB169 | 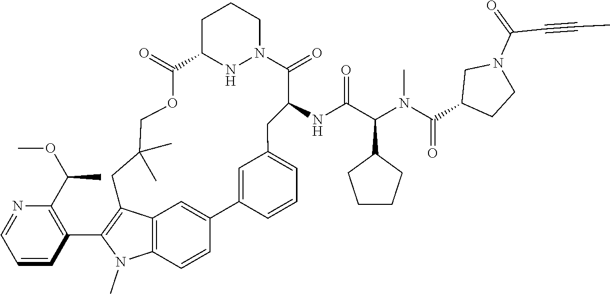 |
| BB170 | 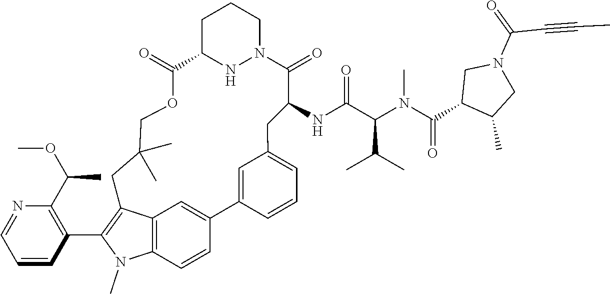 |
| BB171 | 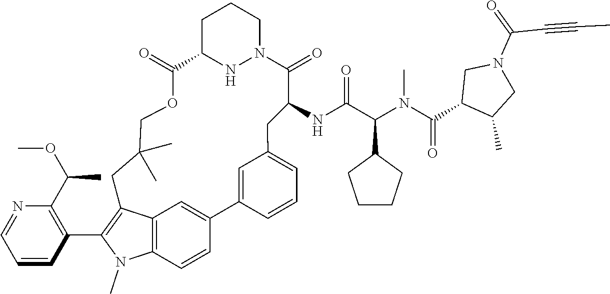 |
| BB172 | 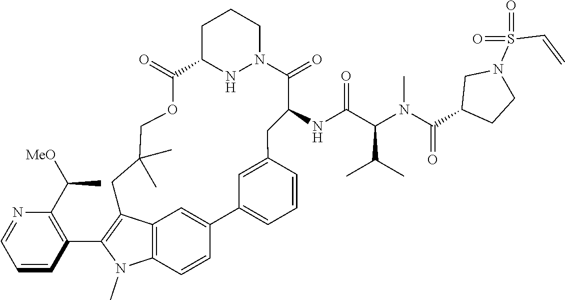 |
| BB173 | 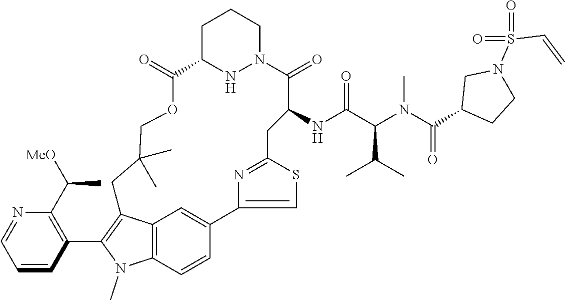 |
| BB174 | 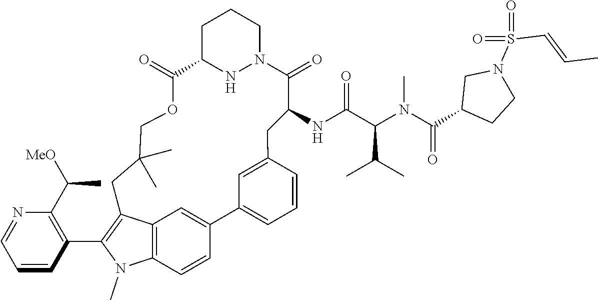 |
| BB175 | 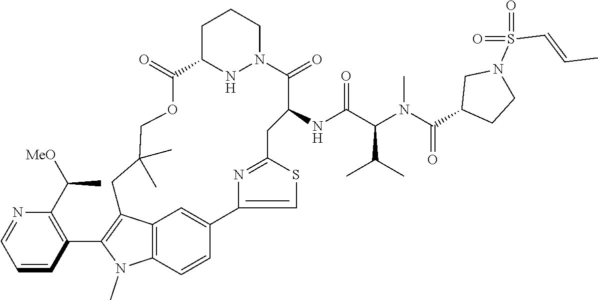 |
| BB176 | 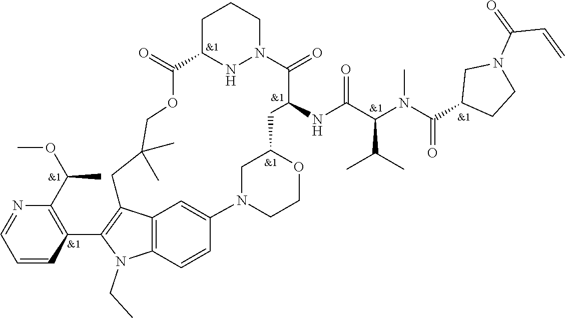 |
| BB177 | 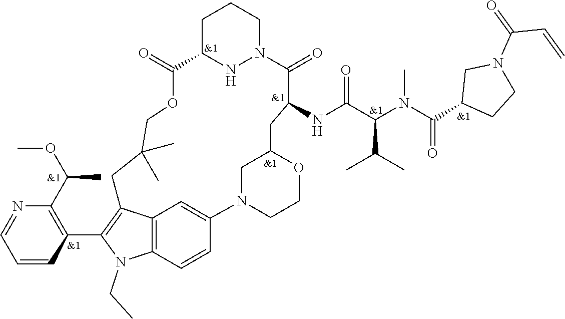 |
| BB178 | 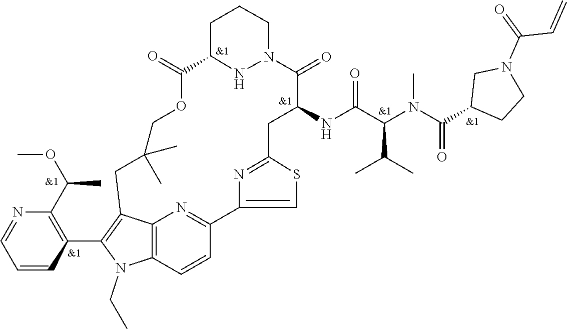 |
| BB179 | 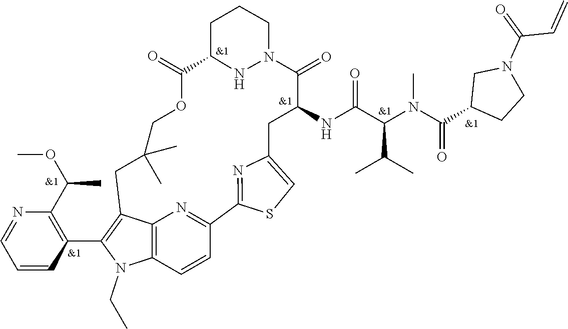 |
| BB180 | 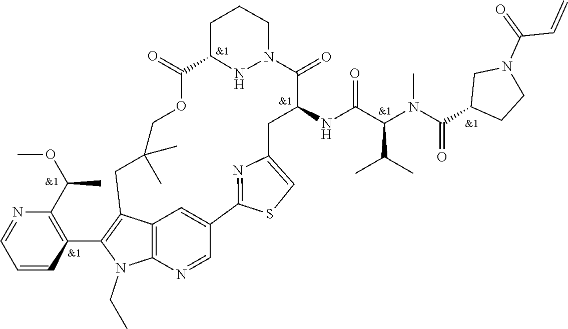 |
| BB181 | 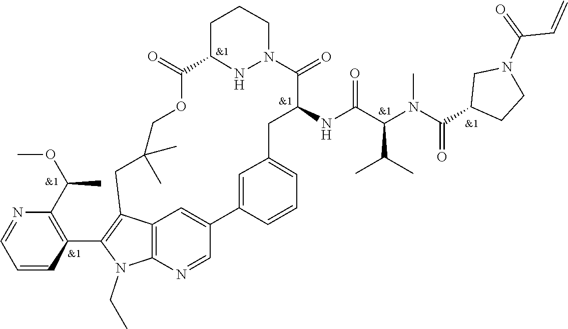 |
| BB182 | 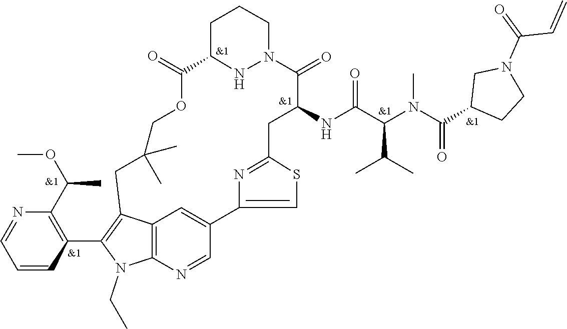 |
| BB183 | 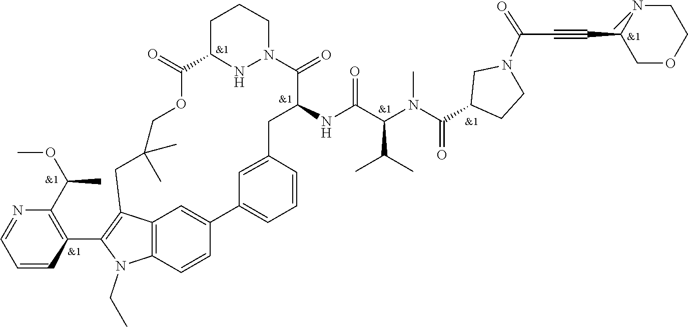 |
| BB184 | 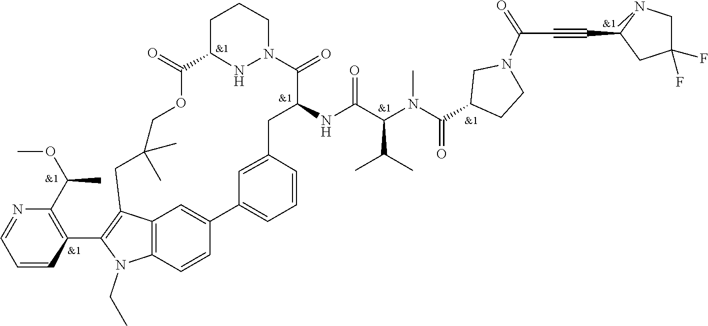 |
| BB185 | 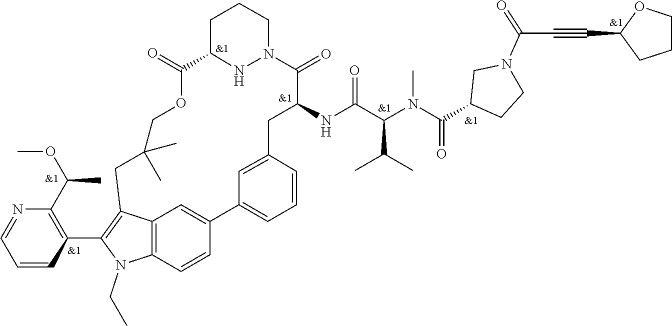 |
| BB186 | 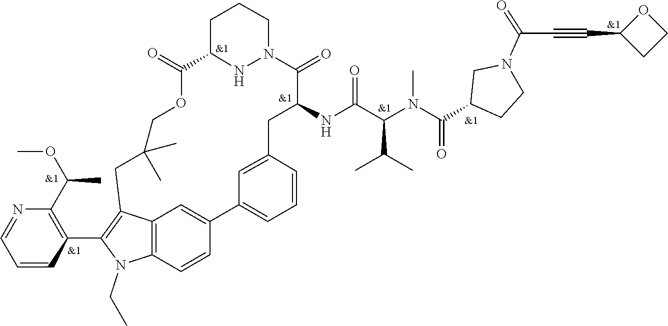 |
| BB187 | 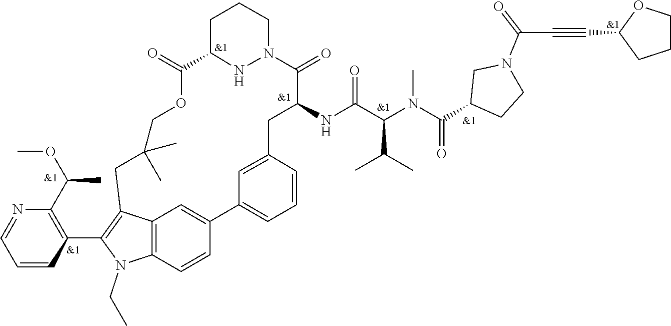 |
| BB188 | 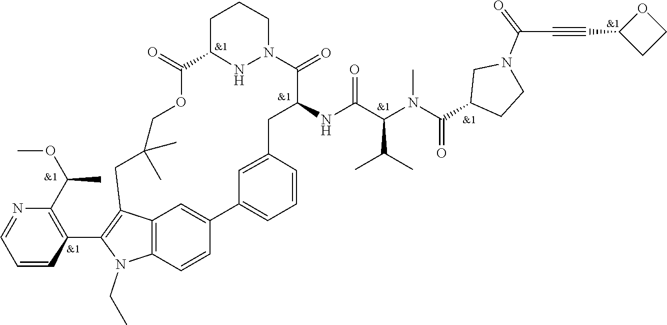 |
| BB189 | 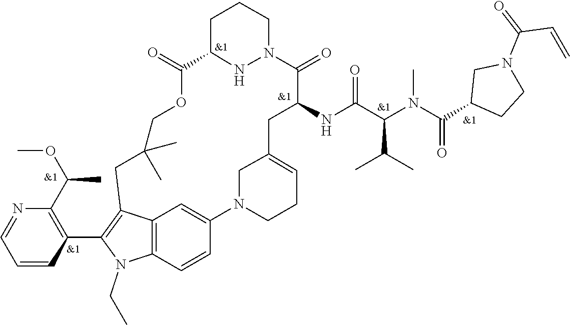 |
| BB190 | 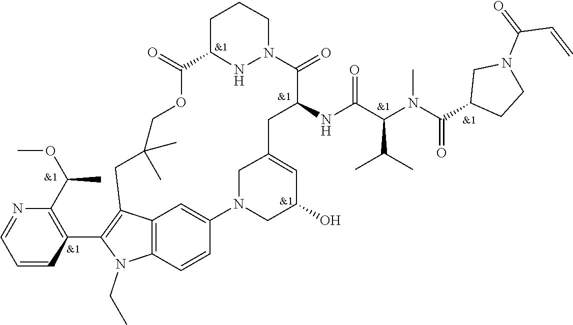 |
| BB191 | 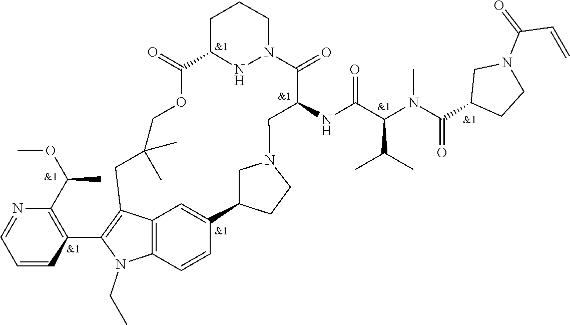 |
| BB192 | 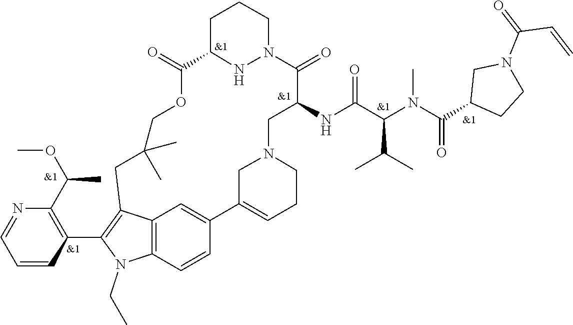 |
| BB194 | 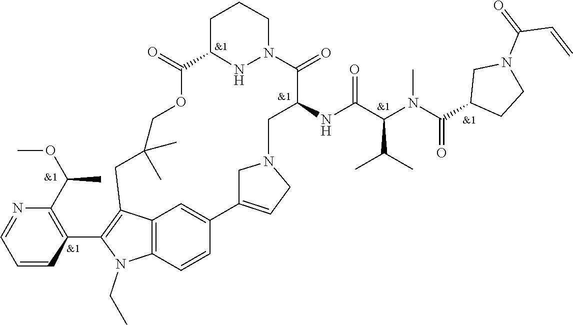 |
| BB195 | 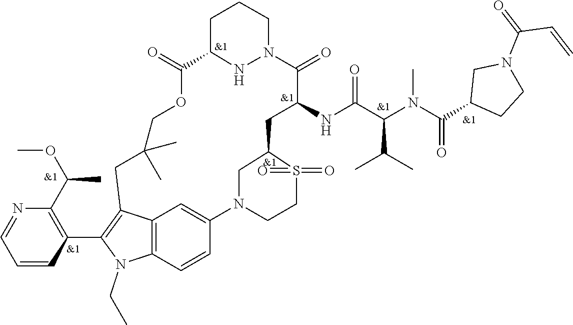 |
| BB196 | 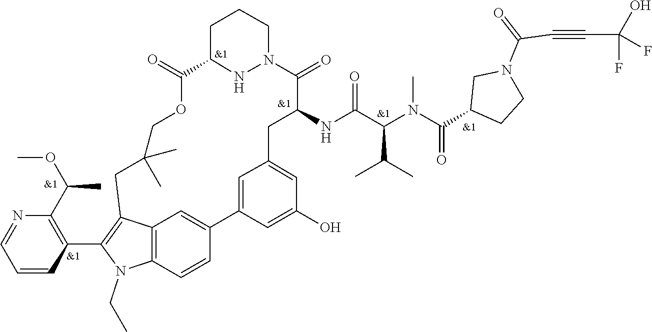 |
| BB197 | 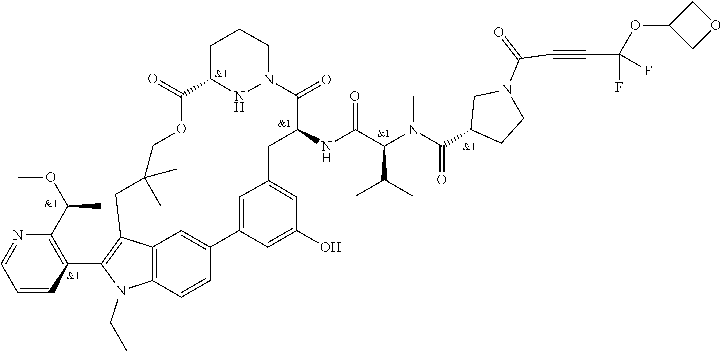 |
| BB198 | 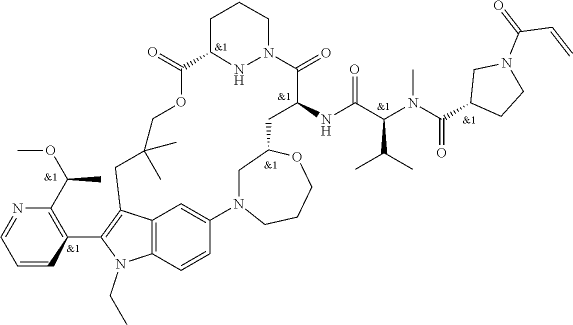 |
| BB199 | 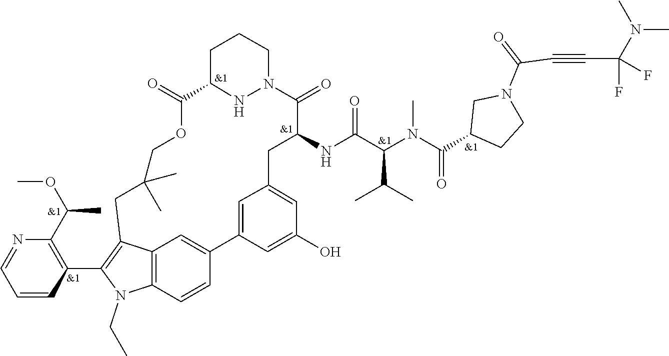 |
| BB200 | 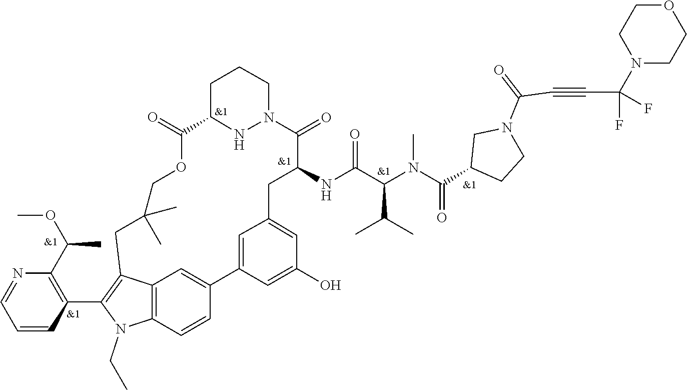 |
| BB201 | 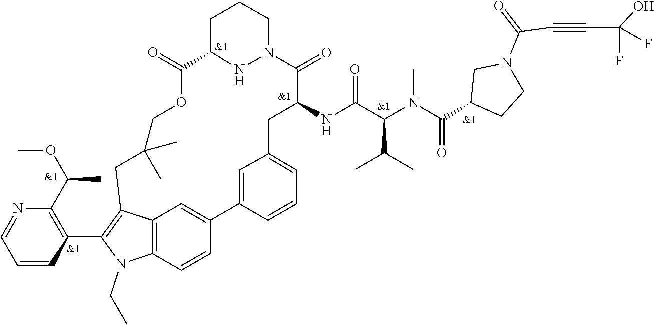 |
| BB202 | 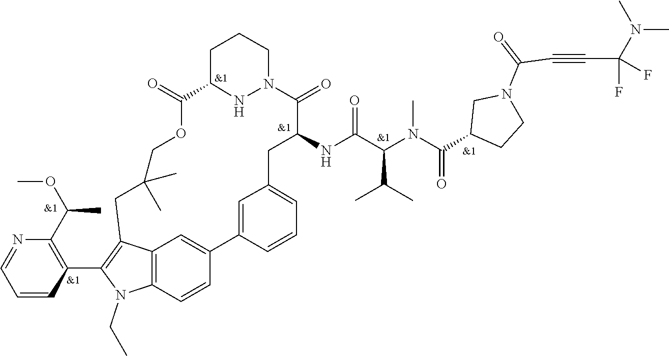 |
| BB203 | 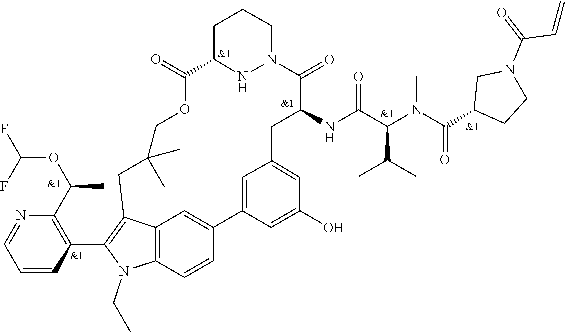 |
| BB204 | 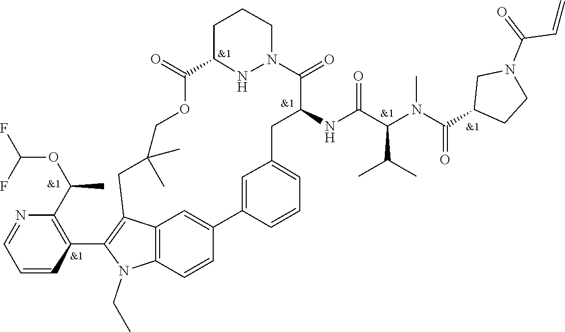 |
| BB205 | 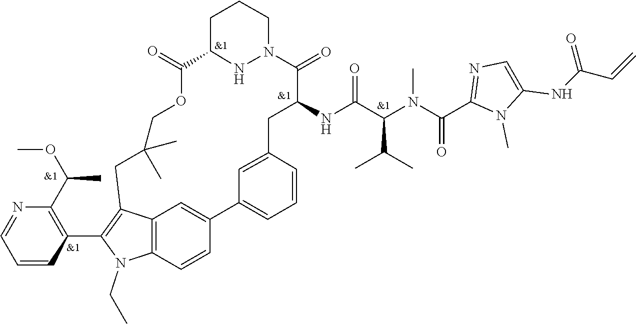 |
| BB206 | 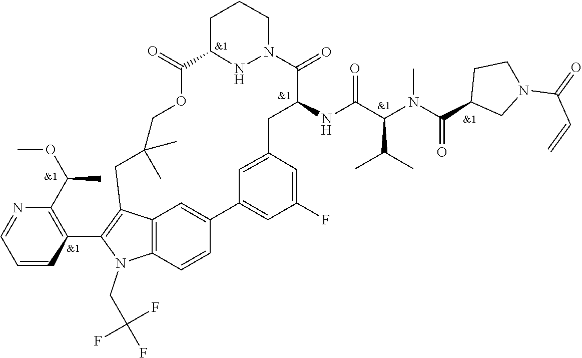 |
| BB207 | 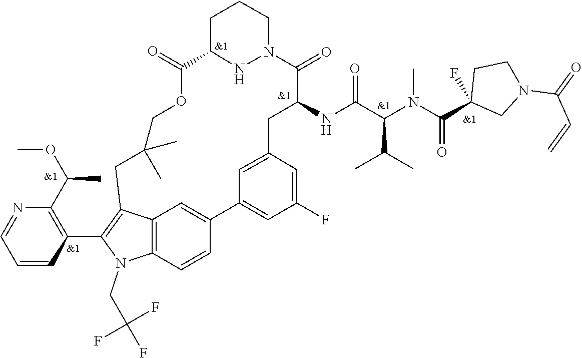 |
| BB208 | 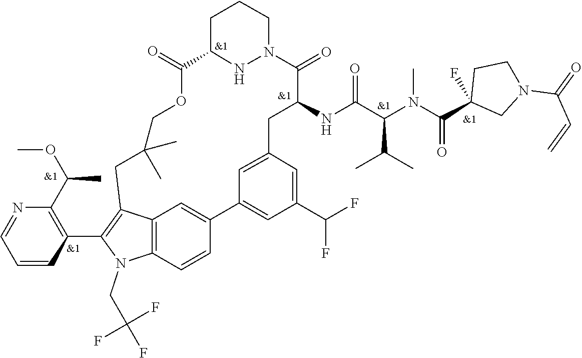 |
| BB209 | 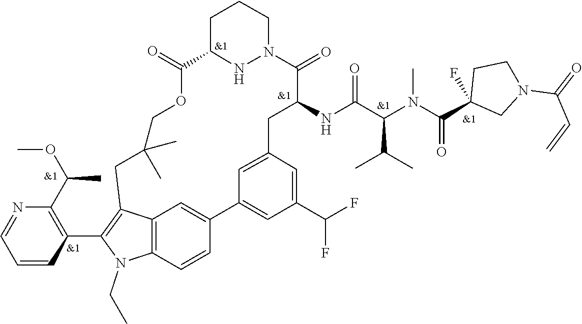 |
| BB210 | 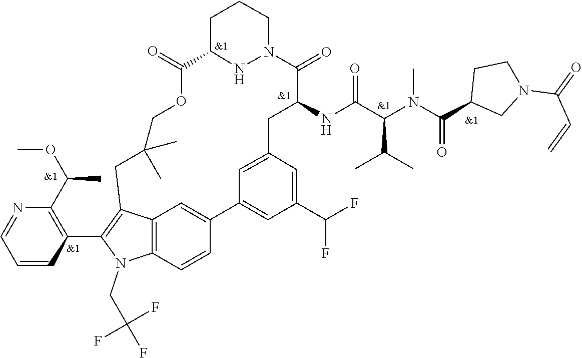 |
| BB211 | 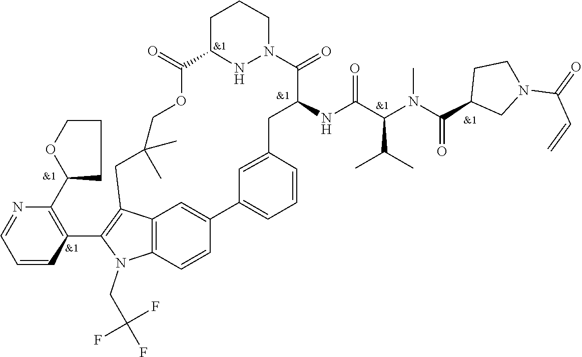 |
| BB212 | 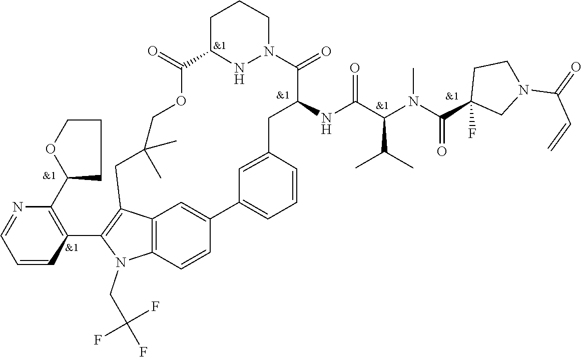 |
| BB213 | 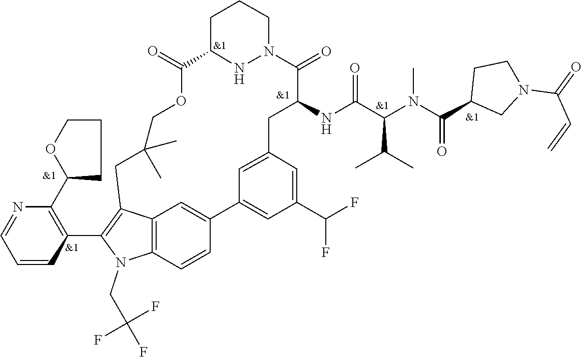 |
| BB214 | 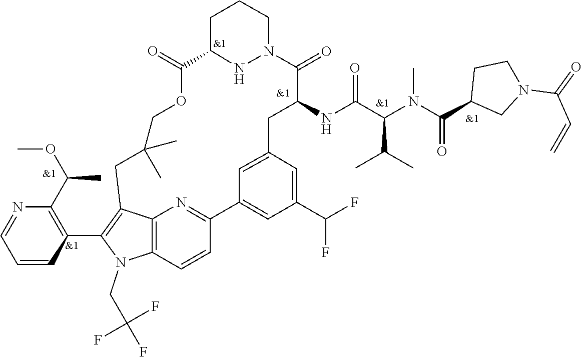 |
| BB215 | 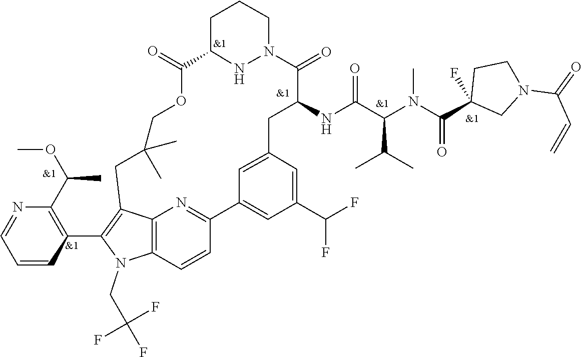 |
| BB216 | 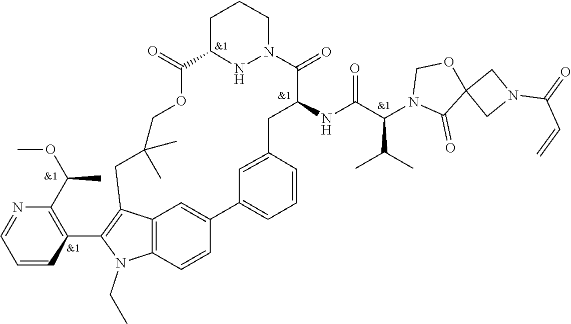 |
| BB217 | 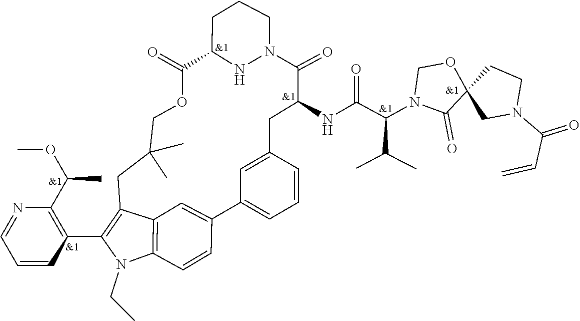 |
| BB218 | 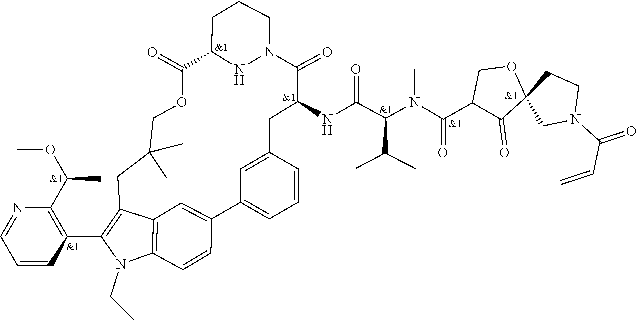 |
| BB219 | 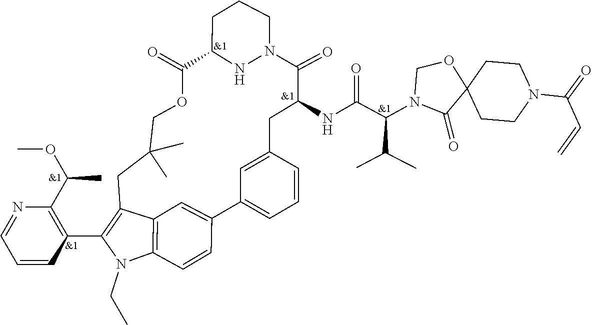 |
| BB220 | 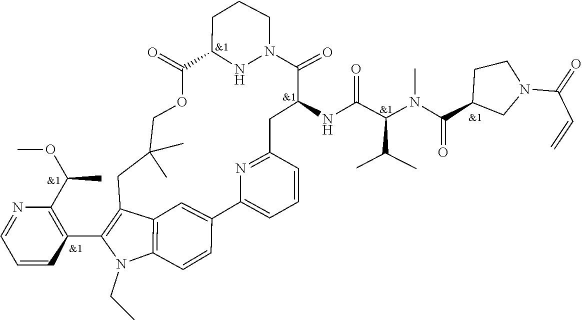 |
| BB221 | 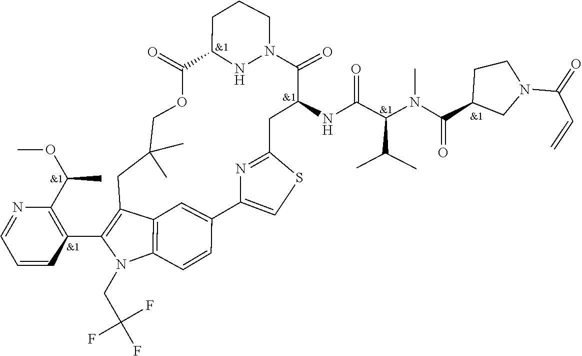 |
| BB222 | 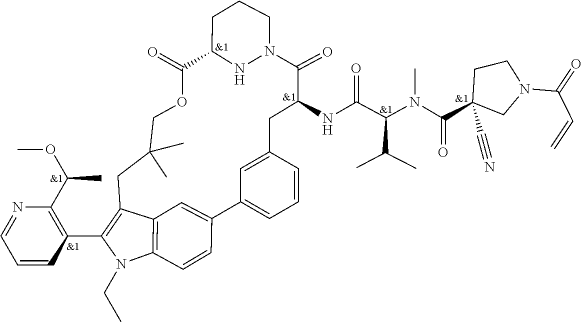 |
| BB223 | 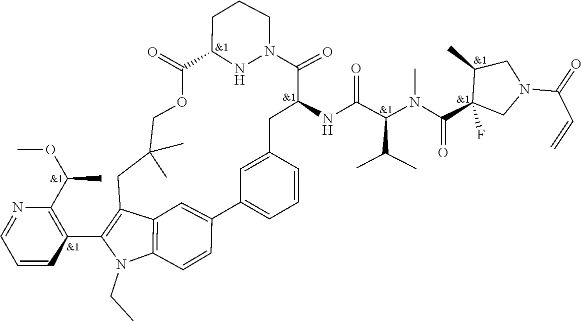 |
| BB224 | 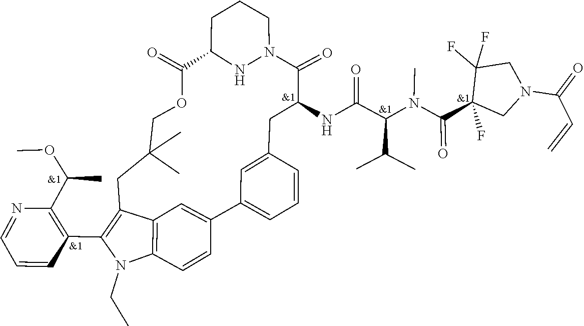 |
| BB225 | 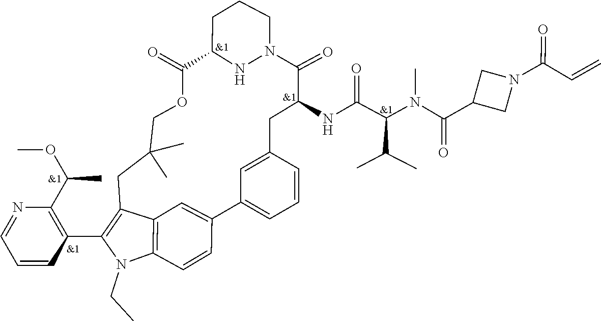 |
| BB226 | 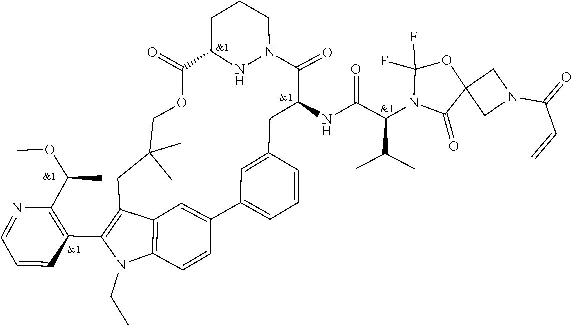 |
| BB227 | 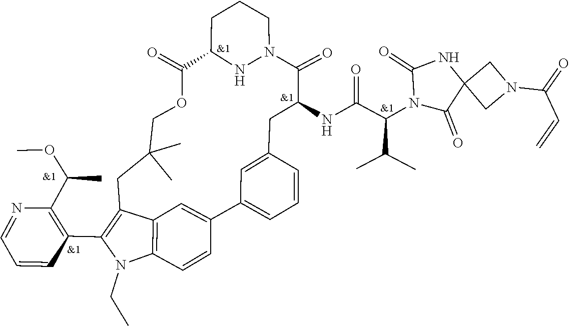 |
| BB228 | 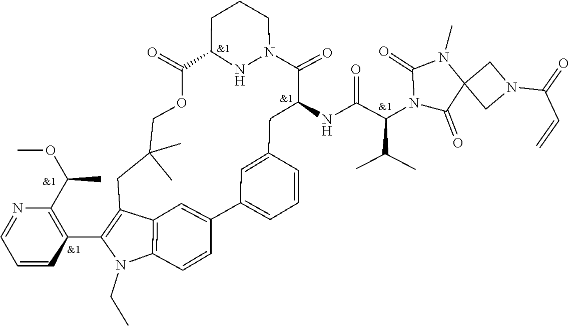 |
| BB229 | 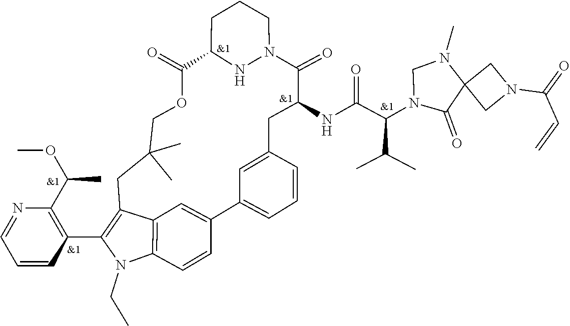 |
| BB230 | 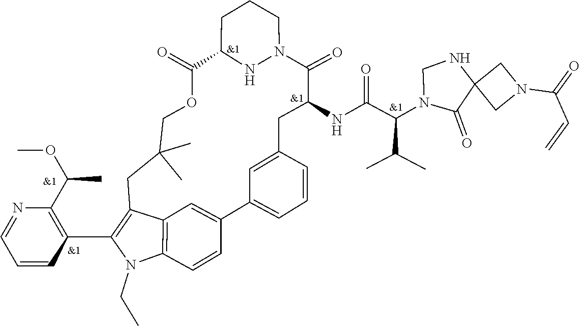 |
| BB231 | 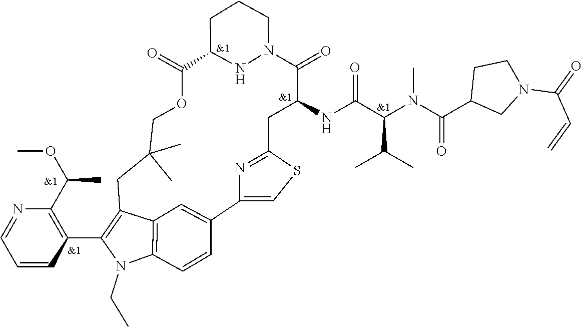 |
| BB232 | 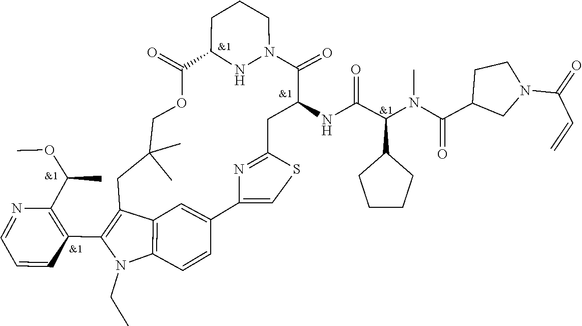 |
| BB233 | 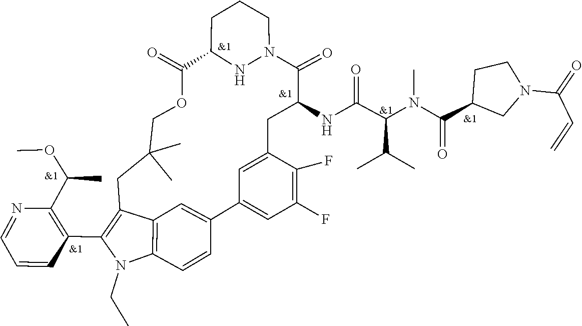 |
| BB234 | 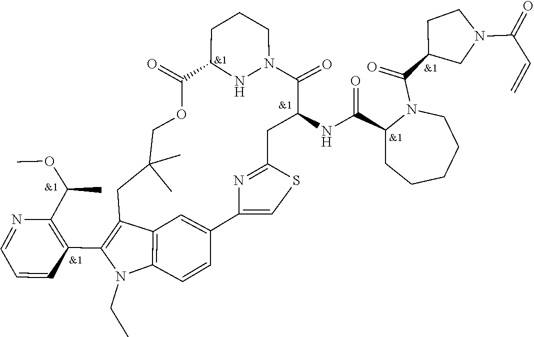 |
| BB235 | 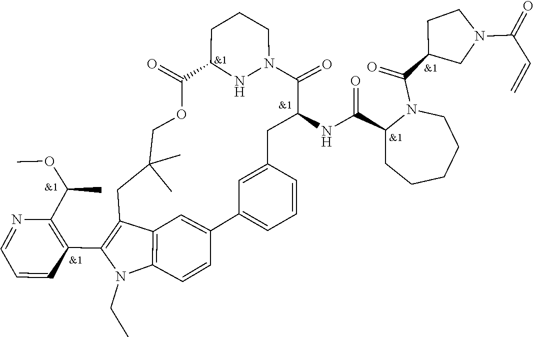 |
| BB236 | 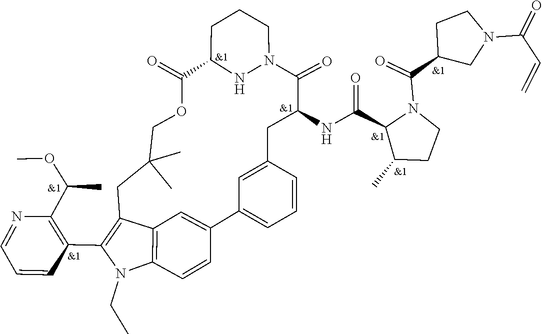 |
| BB237 | 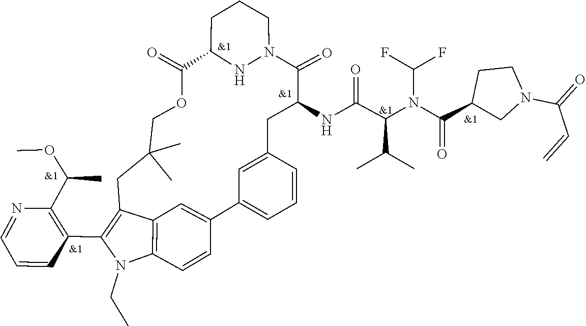 |
| BB238 | 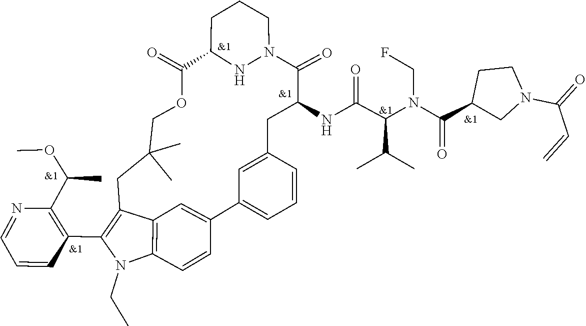 |
| BB239 | 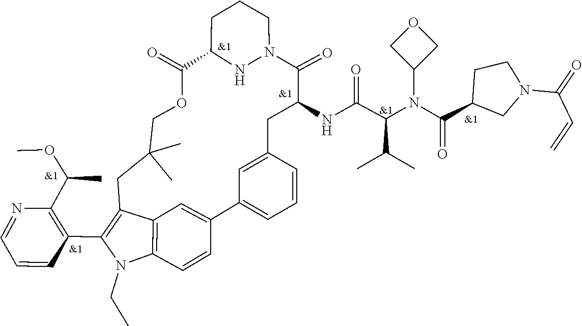 |
| BB240 | 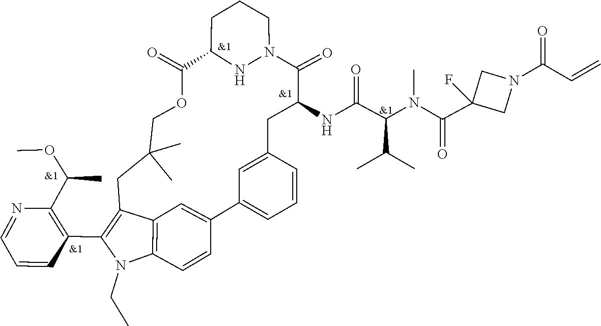 |
| BB241 | 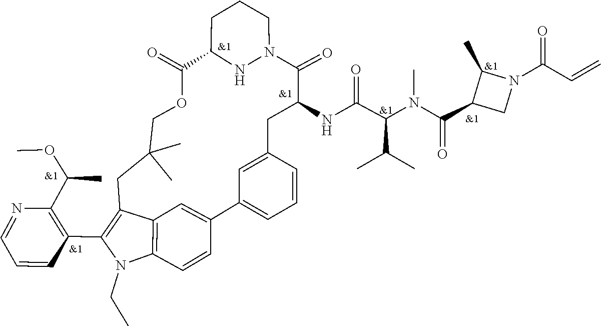 |
| BB242 | 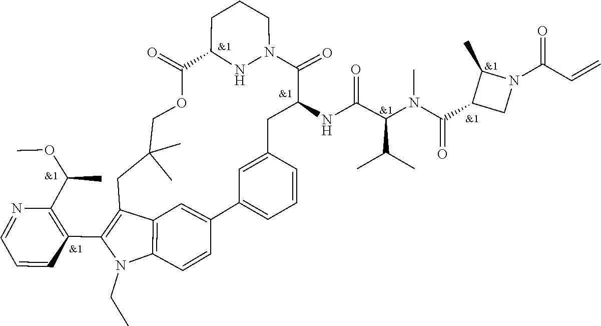 |
| BB243 | 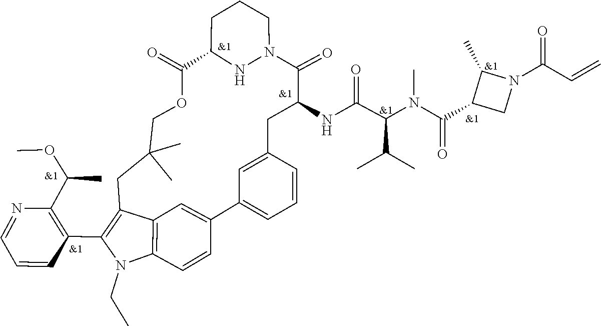 |
| BB244 | 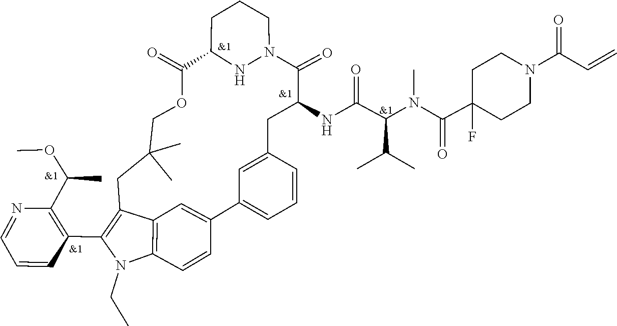 |
| BB245 | 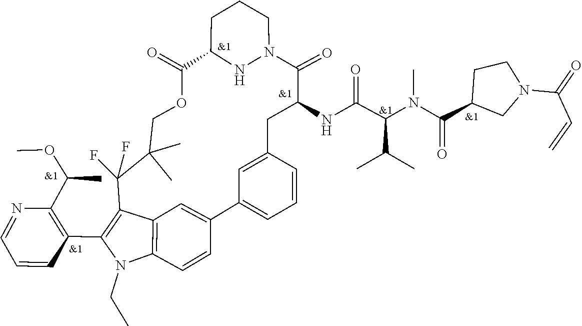 |
| BB246 | 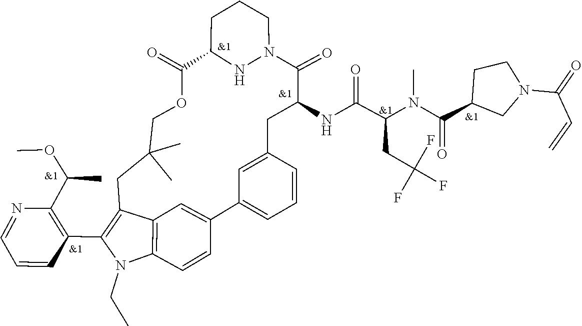 |
| BB247 | 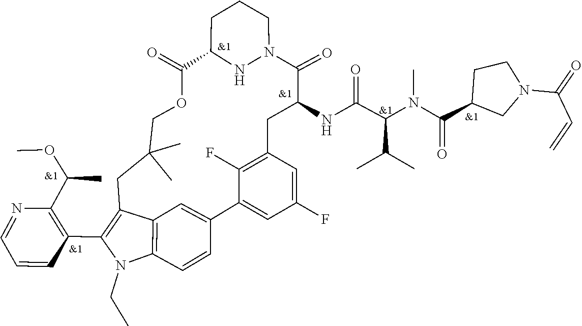 |
| BB248 | 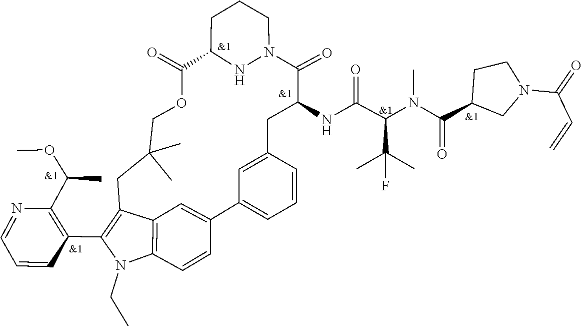 |
| BB249 | 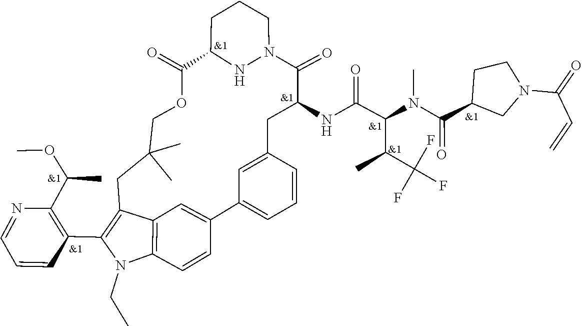 |
| BB250 | 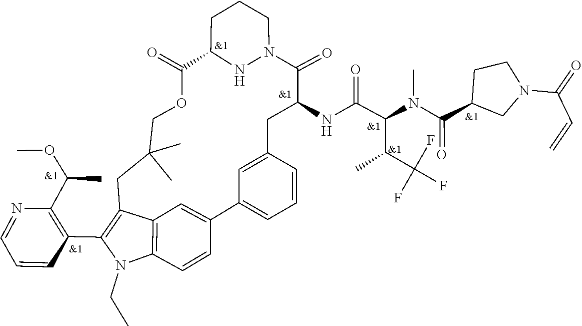 |
| BB251 | 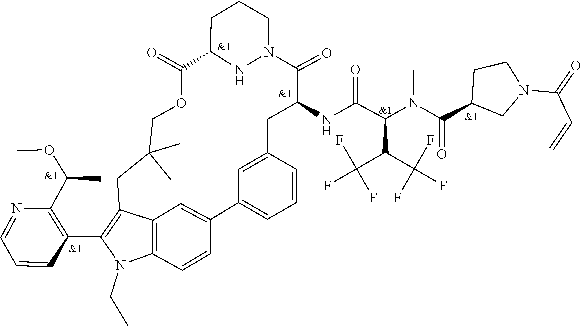 |
| BB252 | 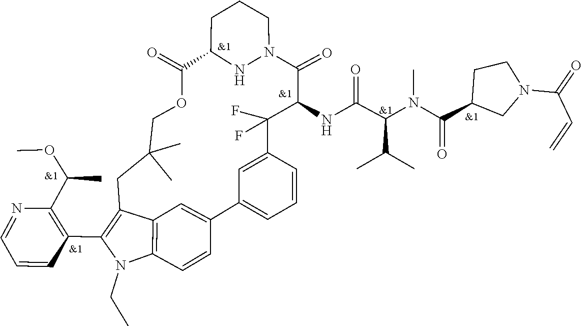 |
| BB253 | 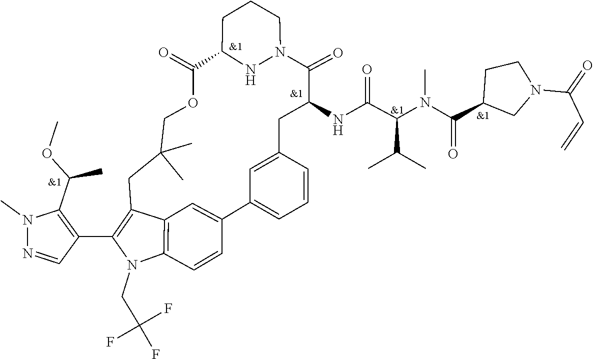 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)COC(=O)[C@@H]3CCCN(N3)C2=O)C(C)C)C1] |
| BB254 | 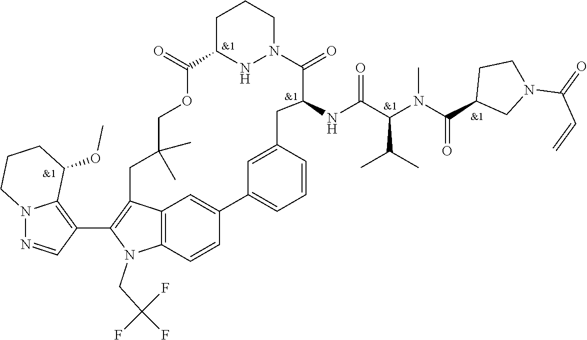 [SMILES: C=CC(=O)N1CC[C@H](C(=O)N(C)[C@H](C(=O)N[C@H]2Cc3cccc(c3)-c3ccc4c(c3)c(c(-c3cnn5c3[C@@H](OC)CCC5)n4CC(F)(F)F)CC(C)(C)COC(=O)[C@@H]3CCCN(N3)C2=O)C(C)C)C1] |
| BB255 | 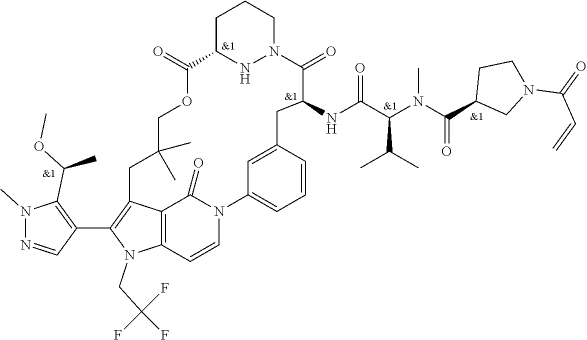 [SMILES: C=CC(=O)N1CC[C@H](C(=O)N(C)[C@H](C(=O)N[C@H]2Cc3cccc(c3)-n3ccc4c(c(c(-c5cnn(C)c5[C@H](C)OC)n4CC(F)(F)F)CC(C)(C)COC(=O)[C@@H]4CCCN(N4)C2=O)c3=O)C(C)C)C1] |
| BB256 | 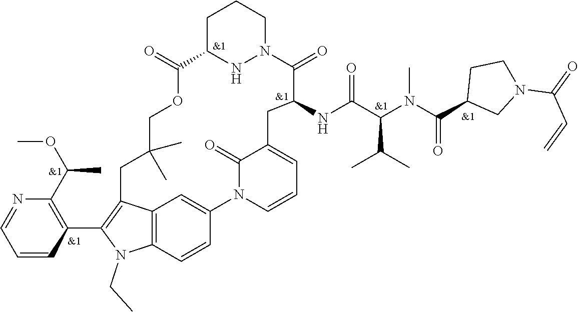 |
| BB257 | 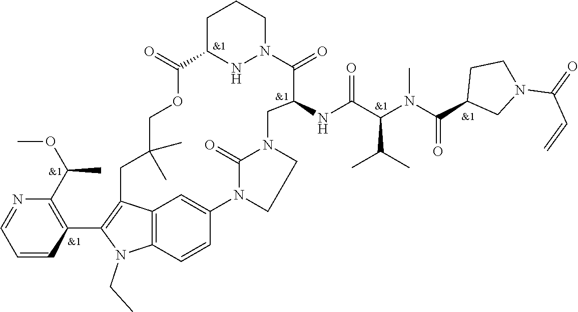 |
| BB258 | 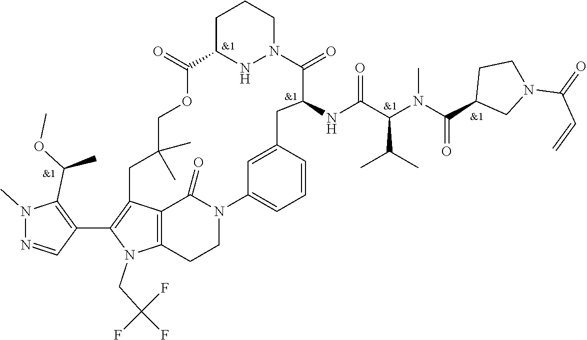 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3=O)C(C)C)C1] |
| BB259 | 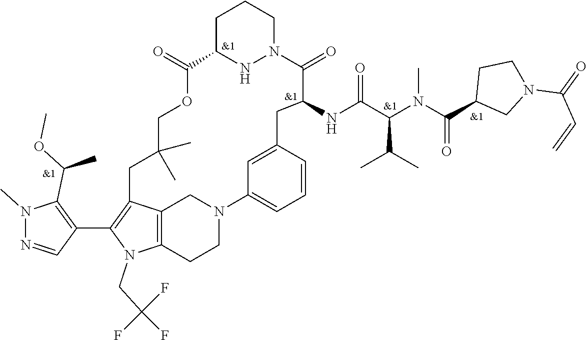 [SMILES: C=CC(=O)N1CC[C@H](C(=O)N(C)[C@H](C(=O)N[C@H]2Cc3cccc(c3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| BB260 | 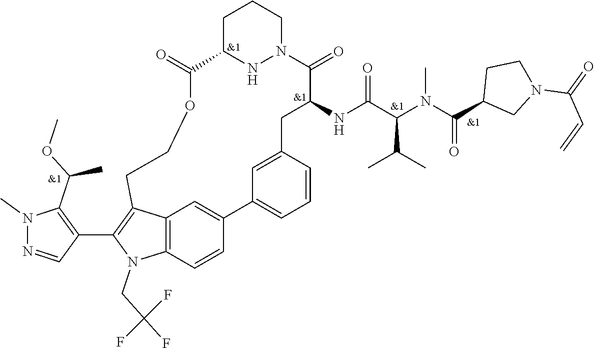 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CCOC(=O)[C@@H]3CCCN(N3)C2=O)C(C)C)C1] |
| BB261 | 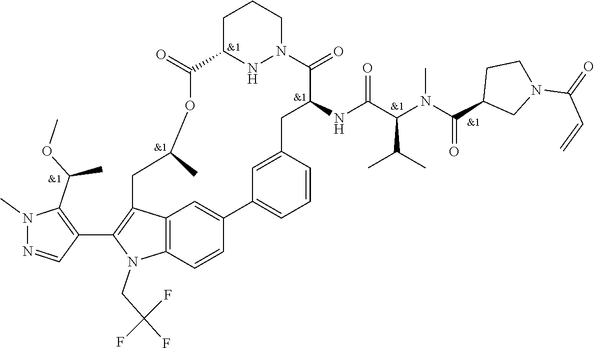 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@H](C)OC(=O)[C@@H]3CCCN(N3)C2=O)C(C)C)C1] |
| BB262 | 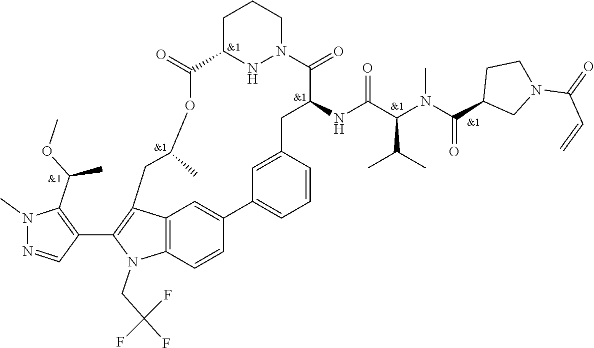 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)C[C@@H](C)OC(=O)[C@@H]3CCCN(N3)C2=O)C(C)C)C1] |
| BB263 | 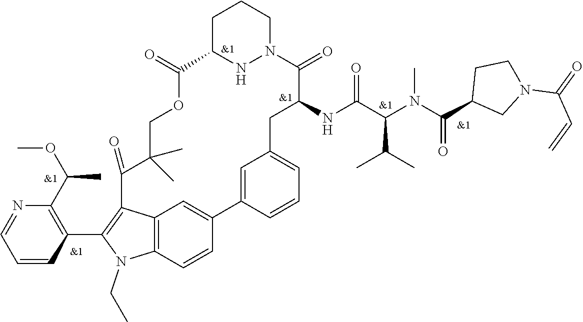 |
| BB264 | 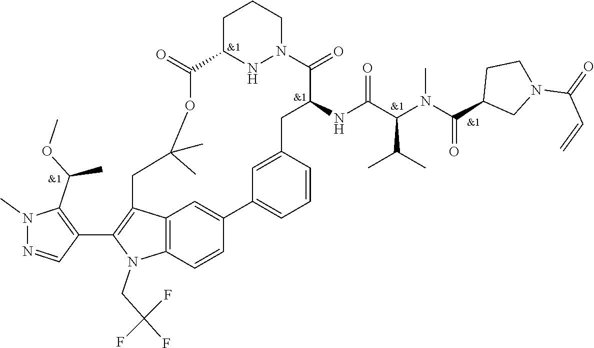 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)OC)n4CC(F)(F)F)CC(C)(C)OC(=O)[C@@H]3CCCN(N3)C2=O)C(C)C)C1] |
| BB265 | 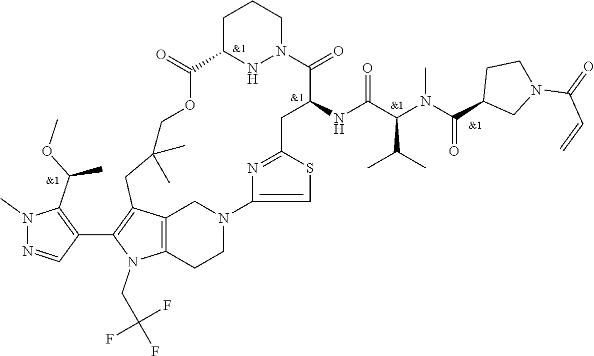 [SMILES: C=CC(=O)N1CC[C@H](C(=O)N(C)[C@H](C(=O)N[C@H]2Cc3nc(cs3)N3CCc4c(c(c(-c5cnn(C)c5[C@H](C)OC)n4CC(F)(F)F)CC(C)(C)COC(=O)[C@@H]4CCCN(N4)C2=O)C3)C(C)C)C1] |
| BB266 | 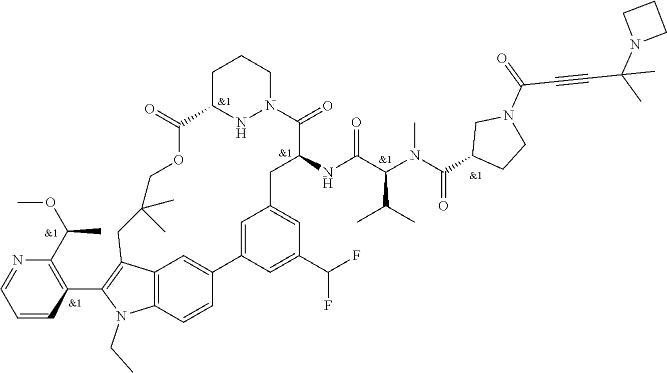 |
| BB267 | 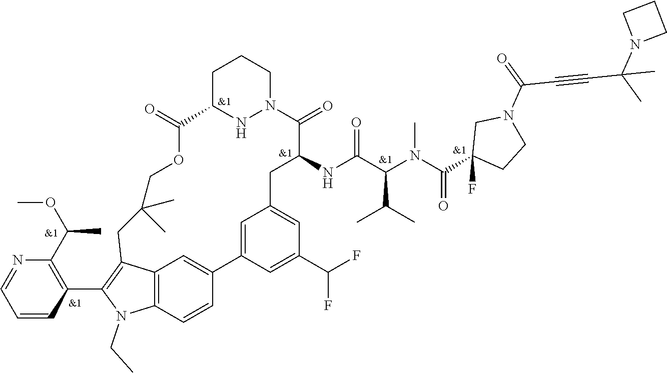 |
| BB268 | 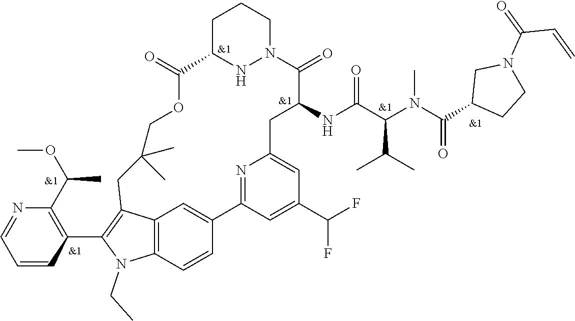 |
| BB269 | 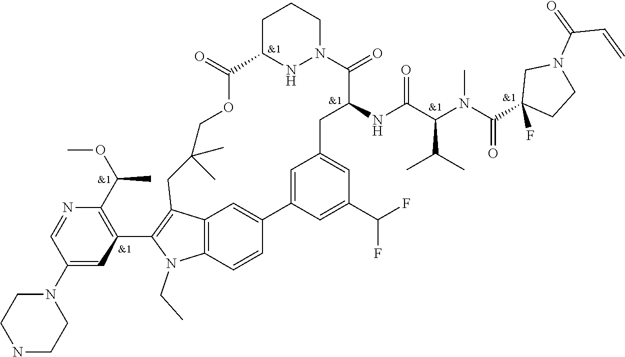 |
| BB270 | 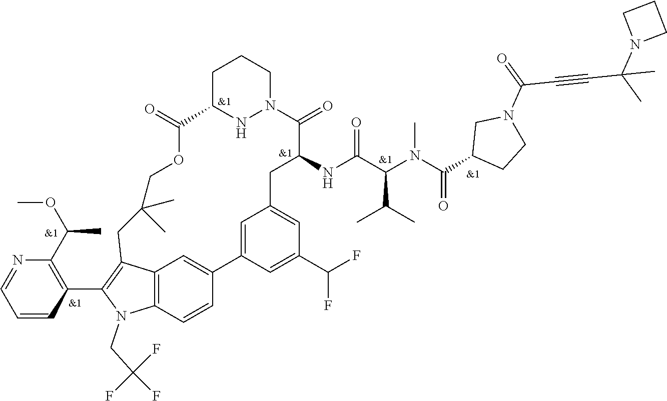 |
| BB271 | 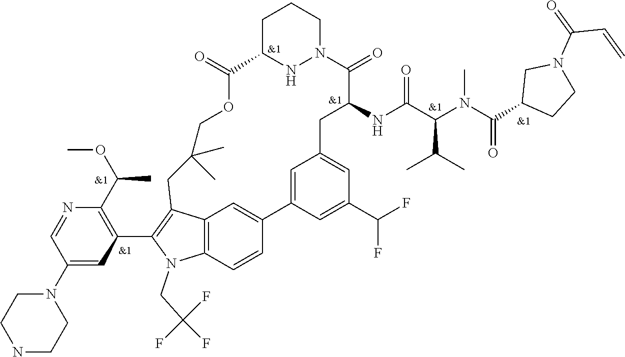 |
| BB272 | 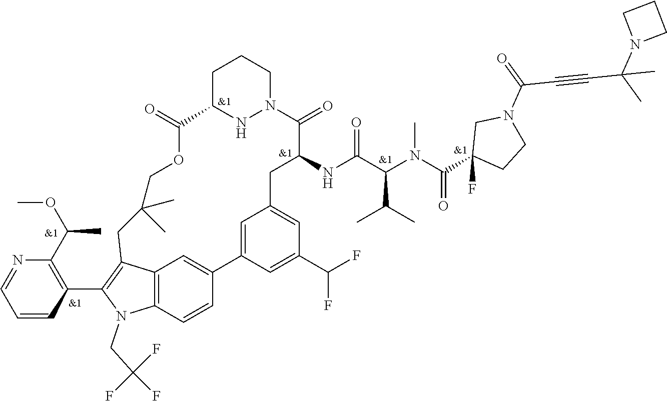 |
| BB273 | 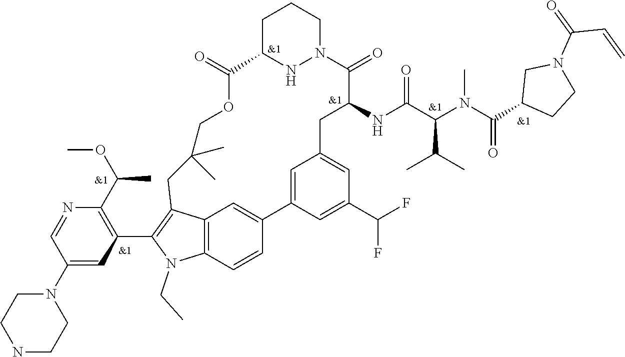 |
| BB274 | 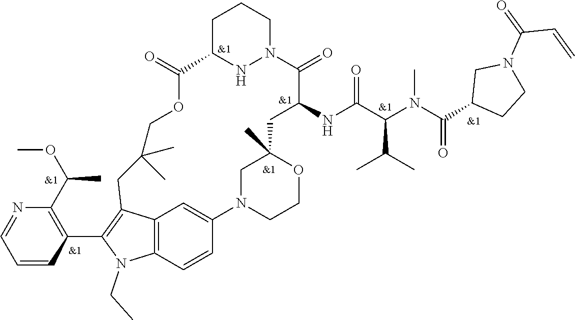 |
| BB275 | 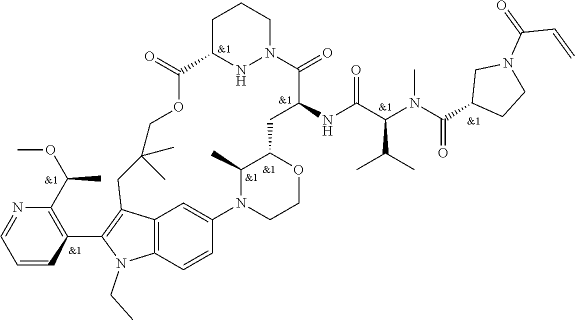 |
| BB276 | 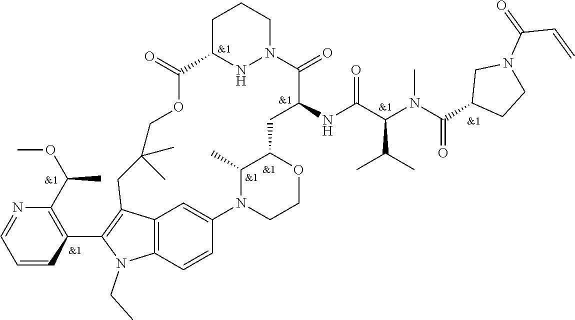 |
| BB277 | 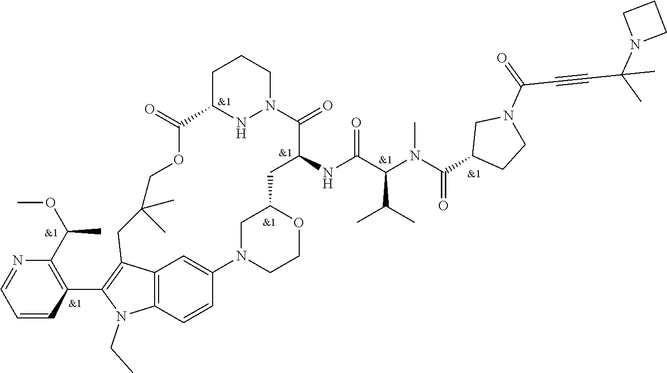 |
| BB278 | 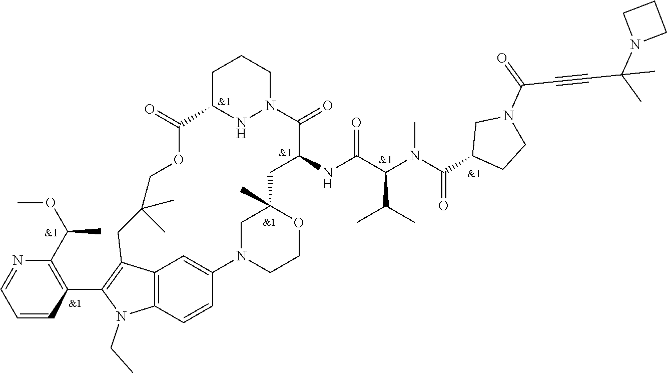 |
| BB279 | 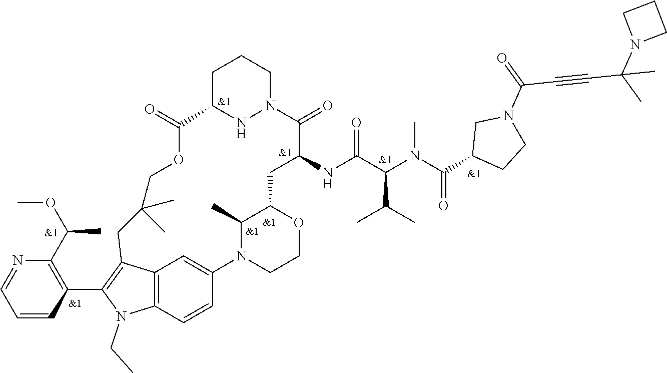 |
| BB280 | 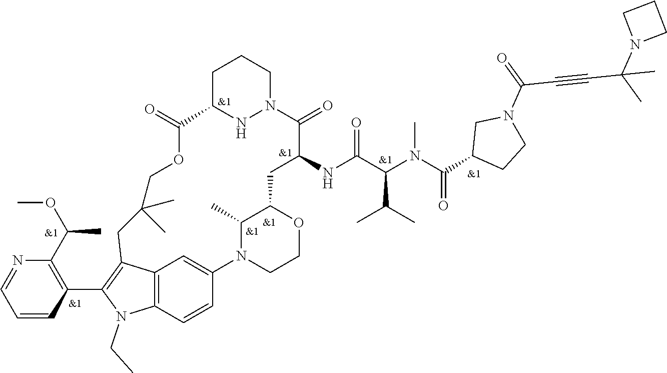 |
| BB282 | 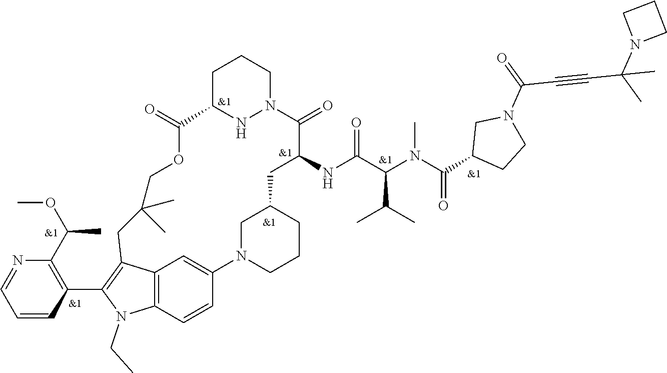 |
| BB283 | 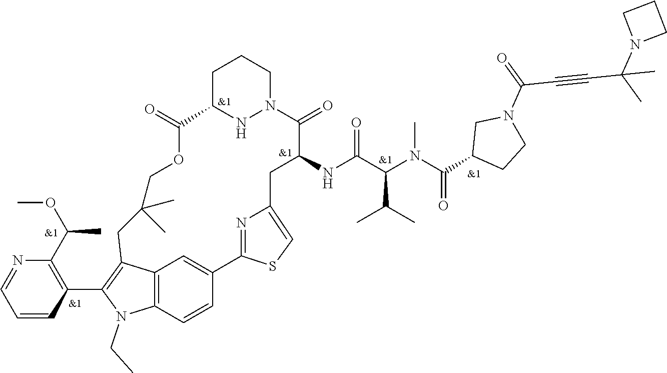 |
| BB284 | 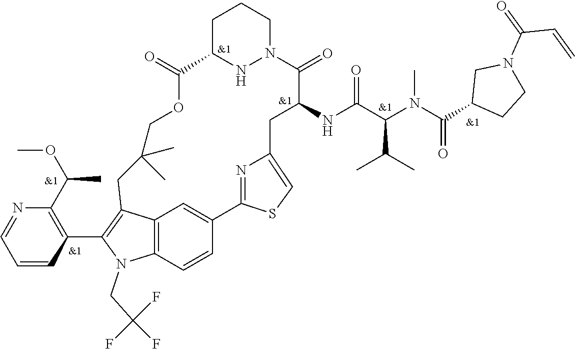 |
| BB285 | 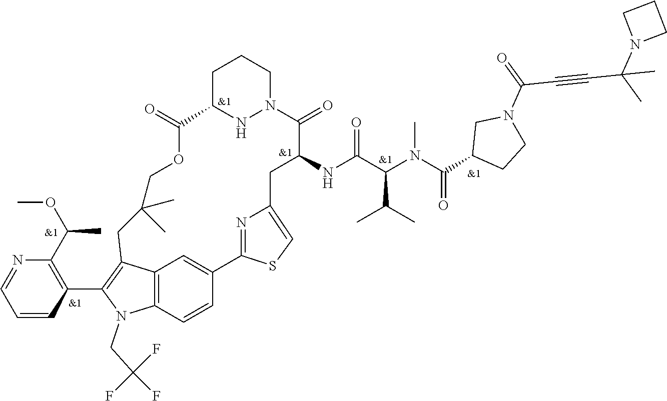 |
| BB286 | 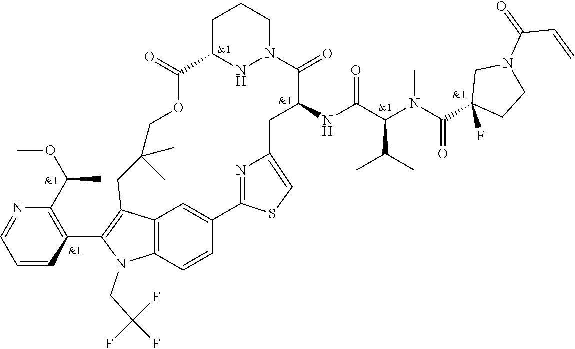 |
| BB287 | 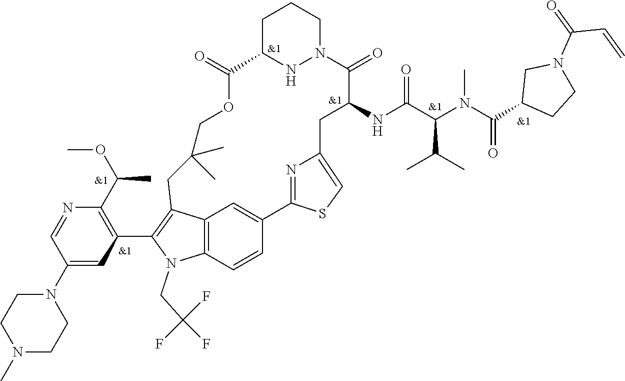 |
| BB288 | 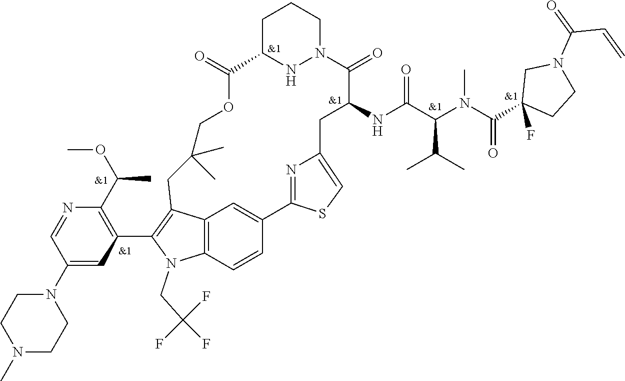 |
| BB289 | 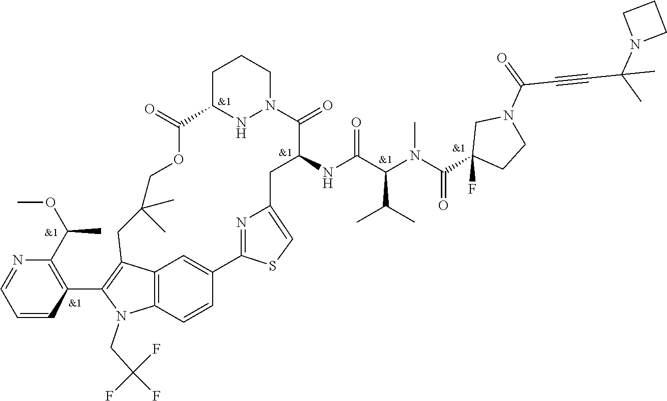 |
| BB290 | 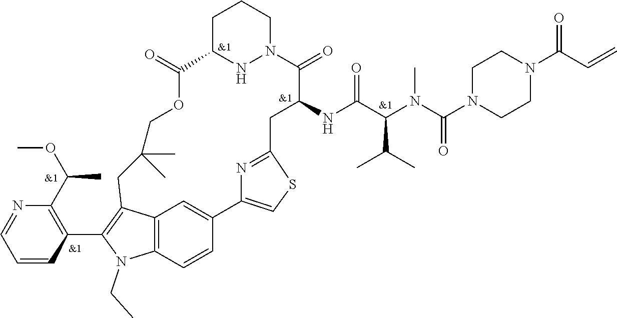 |
| BB291 | 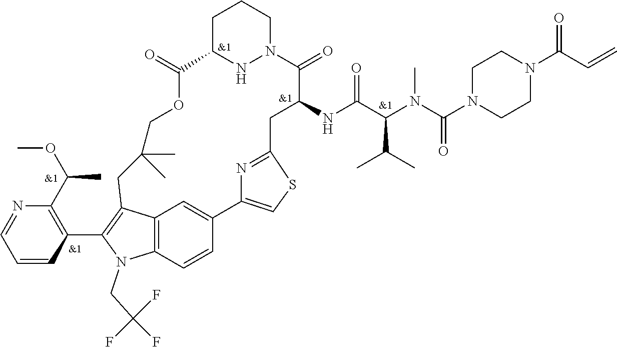 |
| BB292 | 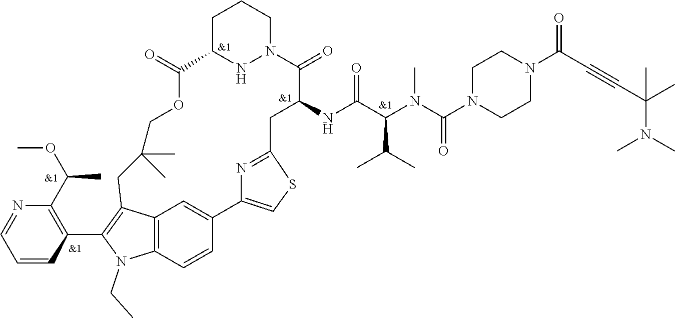 |
| BB293 | 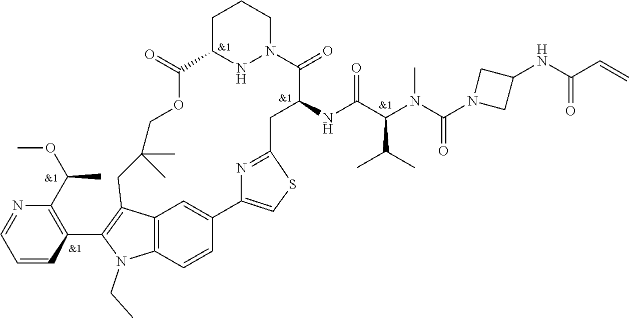 |
| BB294 | 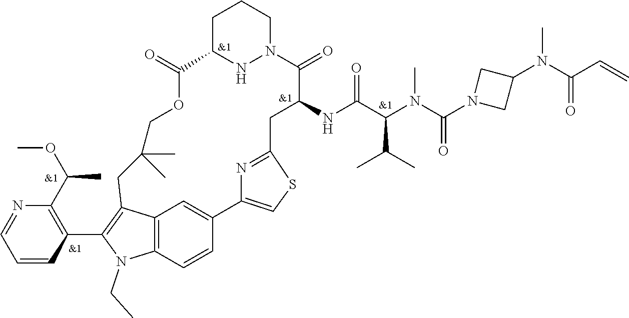 |
| BB295 | 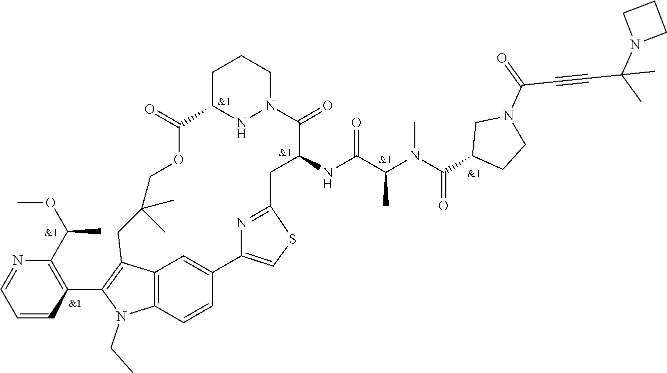 |
| BB296 | 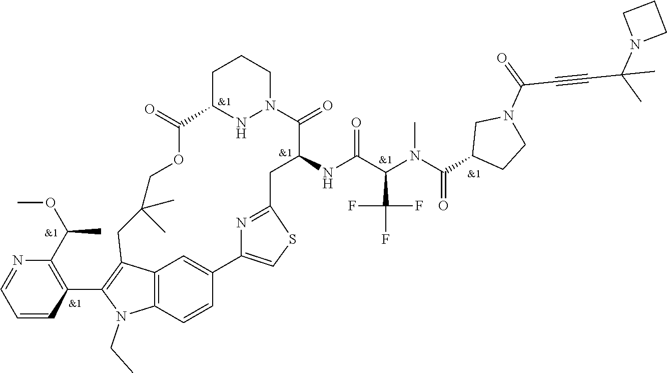 |
| BB297 | 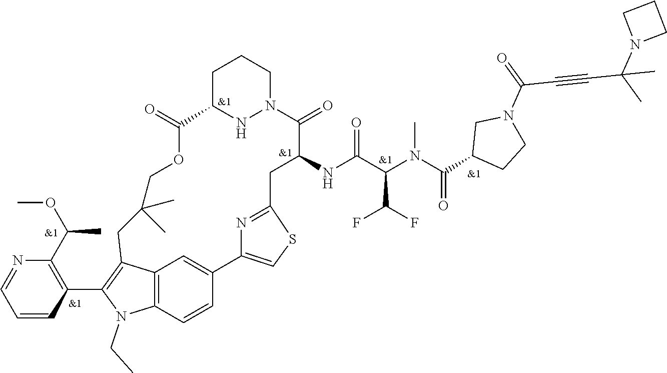 |
| BB298 | 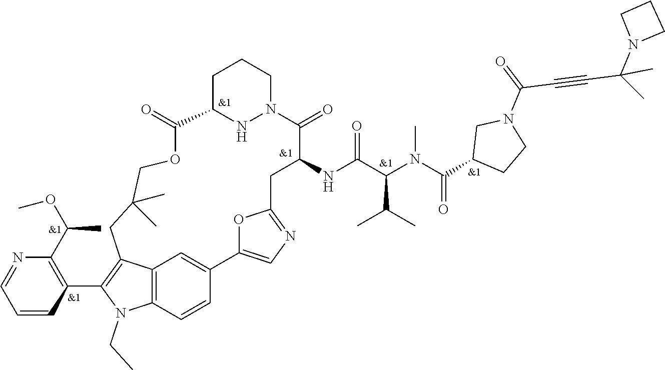 |
| BB299 | 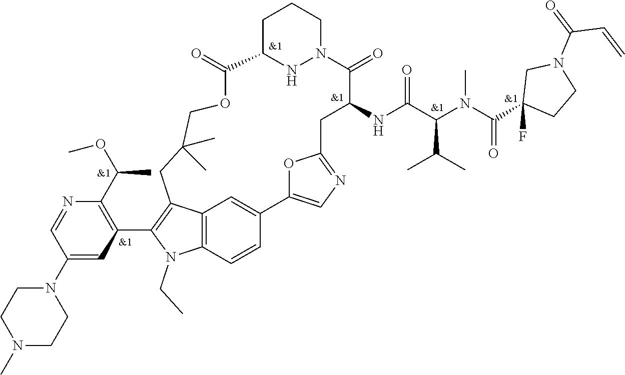 |
| BB300 | 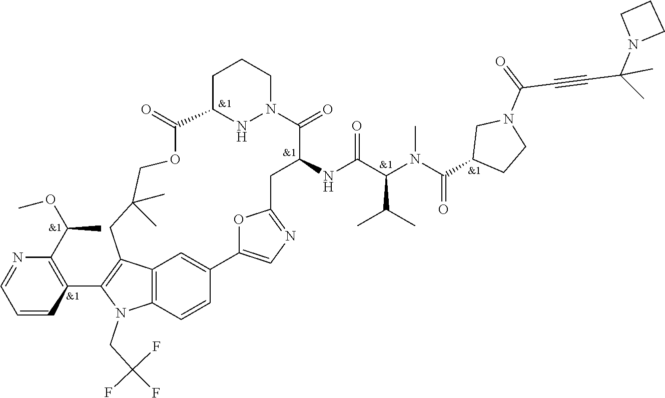 |
| BB301 | 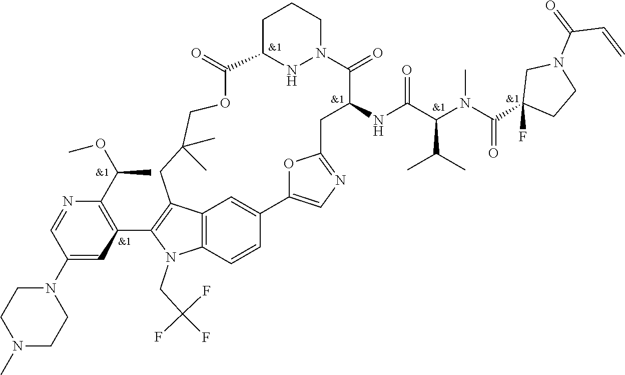 |
| BB302 | 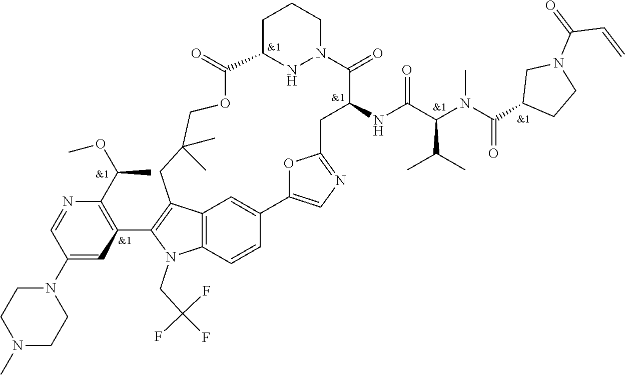 |
| BB303 | 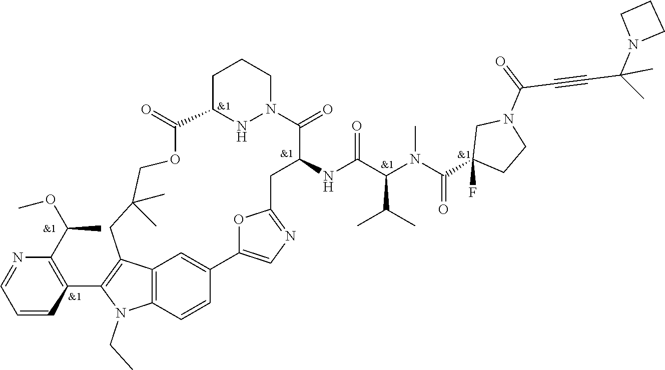 |
| BB304 | 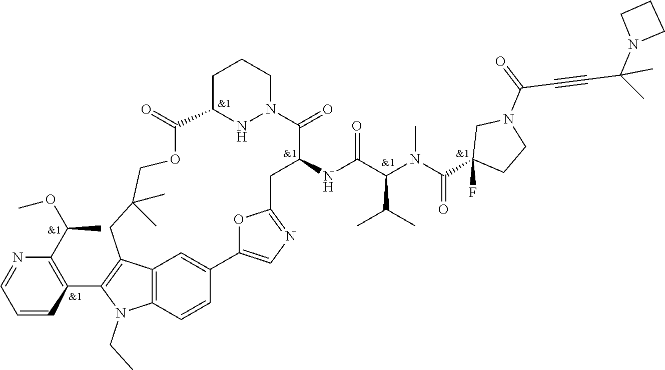 |
| BB305 | 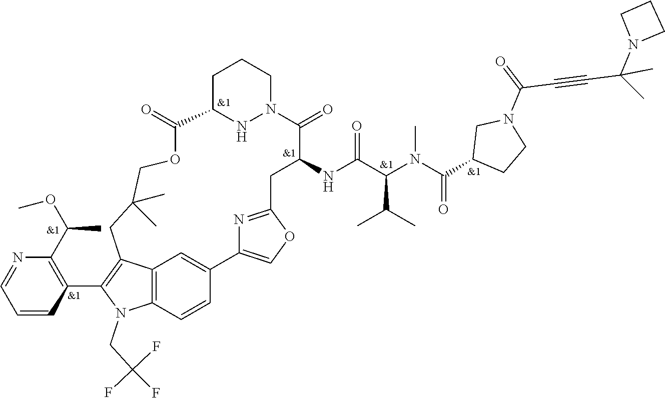 |
| BB306 | 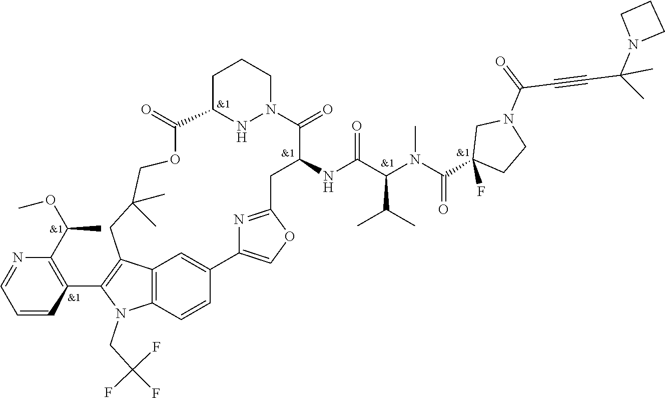 |
| BB307 | 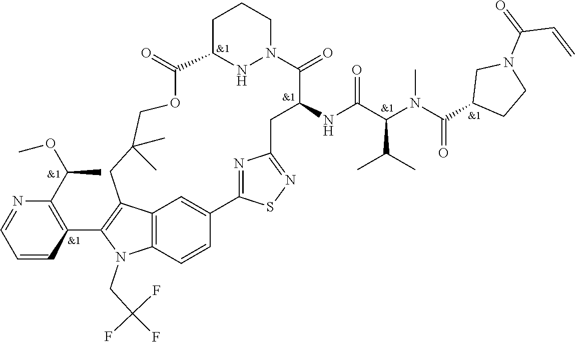 |
| BB308 | 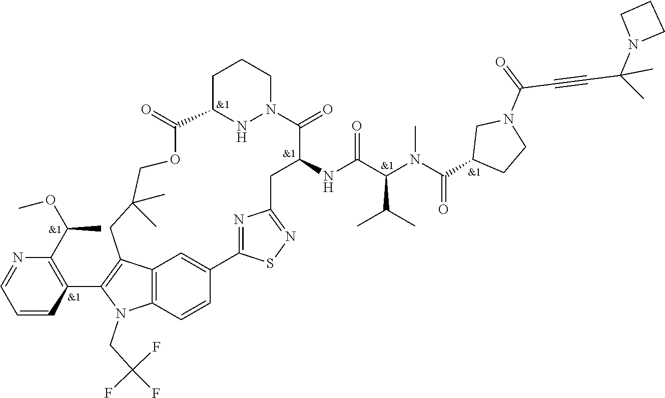 |
| BB309 | 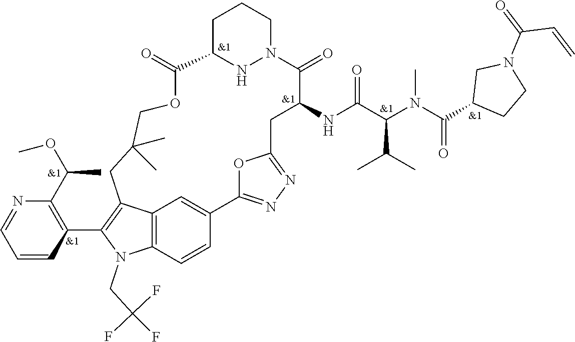 |
| BB310 | 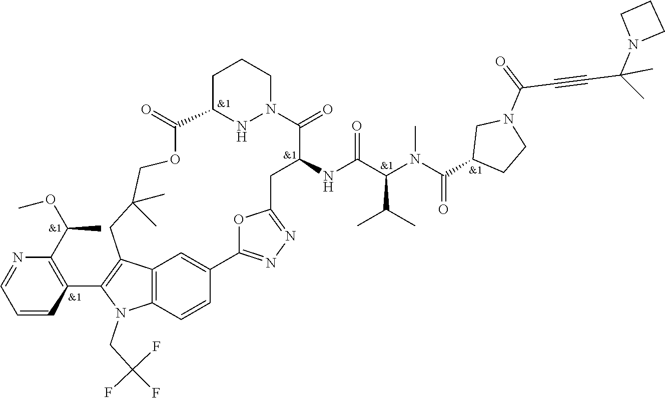 |
| BB311 | 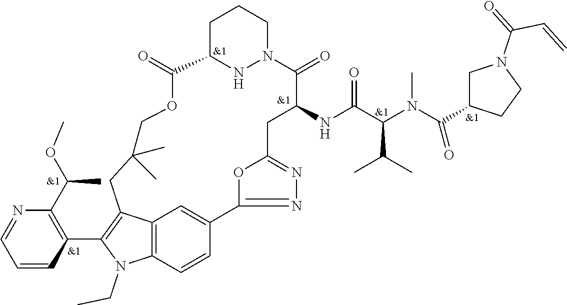 |
| BB312 | 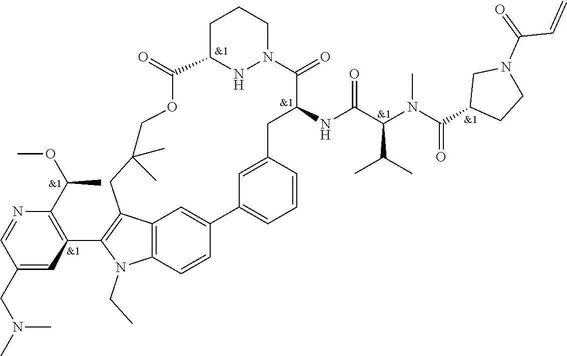 |
| BB313 | 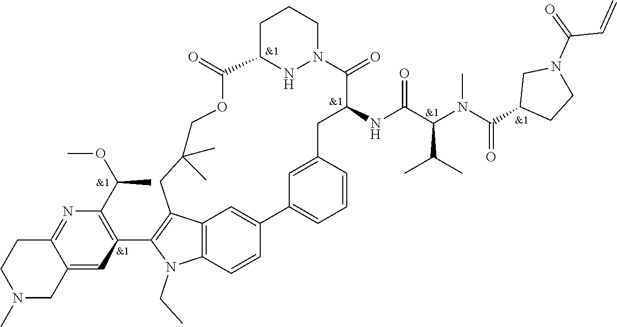 |
| BB314 | 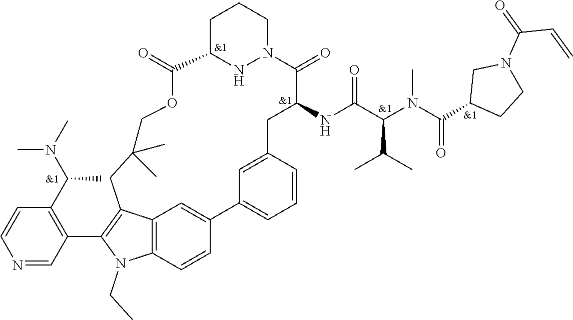 [SMILES: C=CC(=O)N1CC[C@H](C(=O)N(C)[C@H](C(=O)N[C@H]2Cc3cccc(c3)-c3ccc4c(c3)c(c(-c3cnccc3[C@@H](C)N(C)C)n4CC)CC(C)(C)COC(=O)[C@@H]3CCCN(N3)C2=O)C(C)C)C1] |
| BB315 | 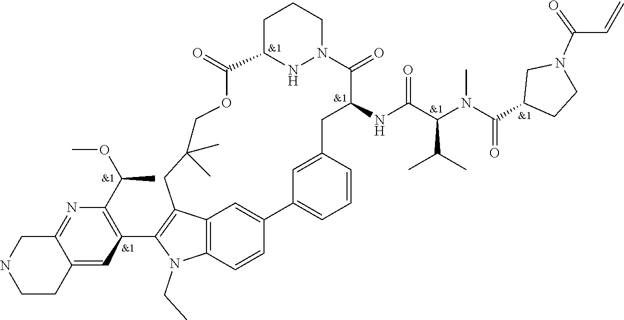 |
| BB316 | 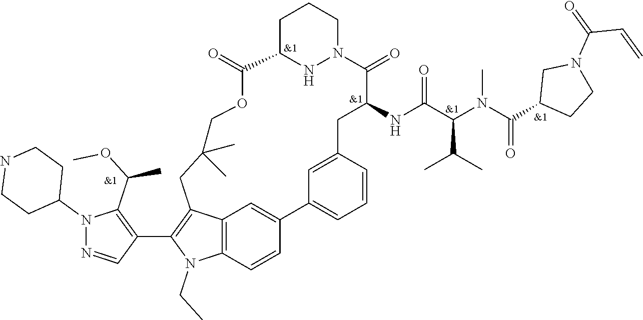 [SMILES: C=CC(=O)N1CC[C@H](C(=O)N(C)[C@H](C(=O)N[C@H]2Cc3cccc(c3)-c3ccc4c(c3)c(c(-c3cnn(C5CCNCC5)c3[C@H](C)OC)n4CC)CC(C)(C)COC(=O)[C@@H]3CCCN(N3)C2=O)C(C)C)C1] |
| BB317 | 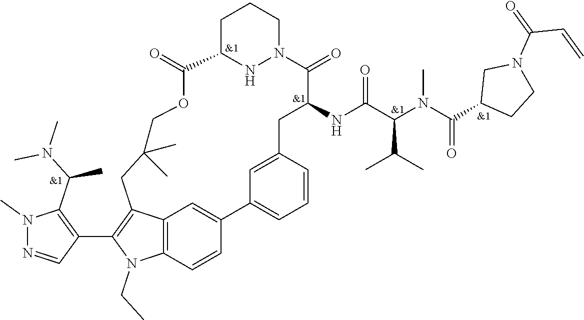 [SMILES: C=CC(=O)N1CC[C@H](C(=O)N(C)[C@H](C(=O)N[C@H]2Cc3cccc(c3)-c3ccc4c(c3)c(c(-c3cnn(C)c3[C@H](C)N(C)C)n4CC)CC(C)(C)COC(=O)[C@@H]3CCCN(N3)C2=O)C(C)C)C1] |
| BB318 | 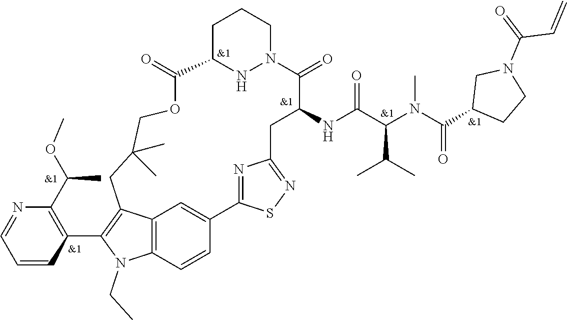 |
| BB319 | 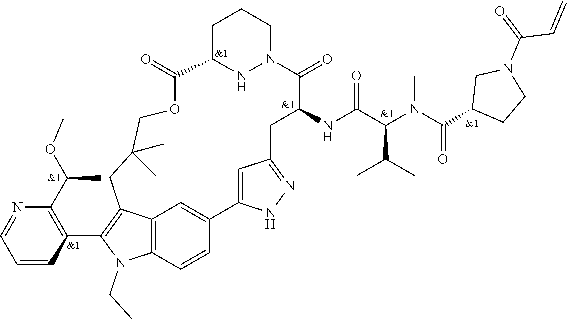 |
| BB320 | 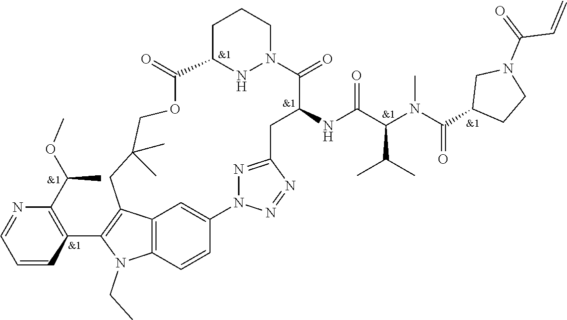 |
| BB321 | 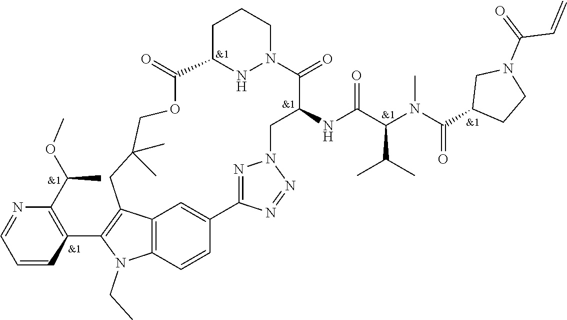 |
| BB322 | 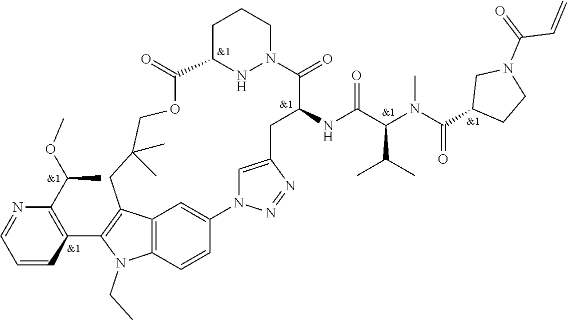 |
| BB323 | 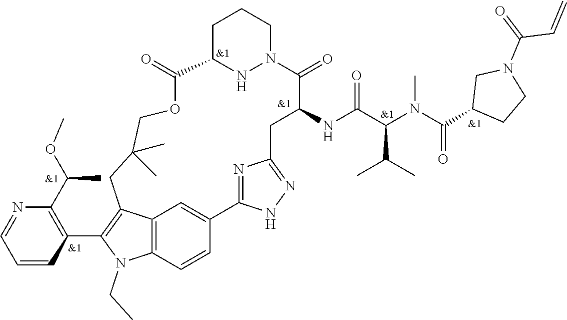 |
| BB324 | 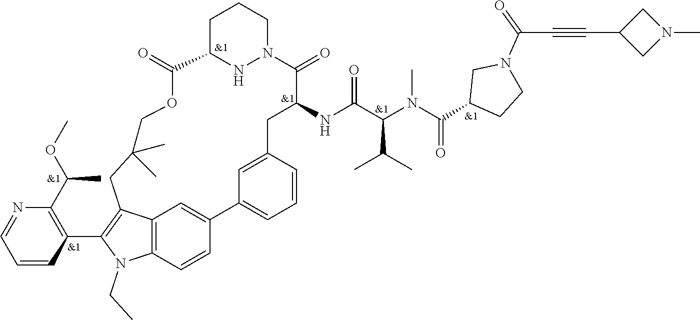 |
| BB325 | 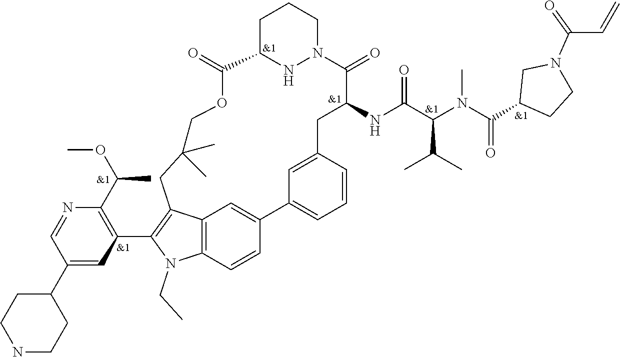 |
| BB326 | 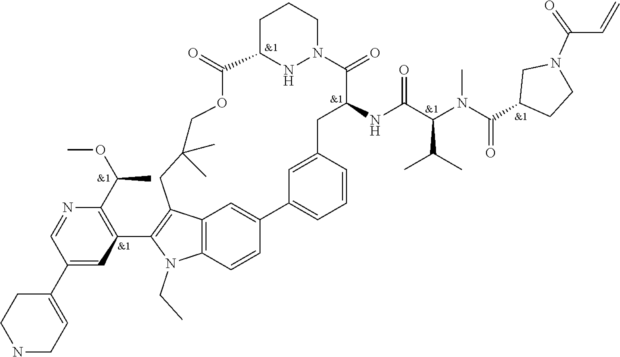 |
| BB327 | 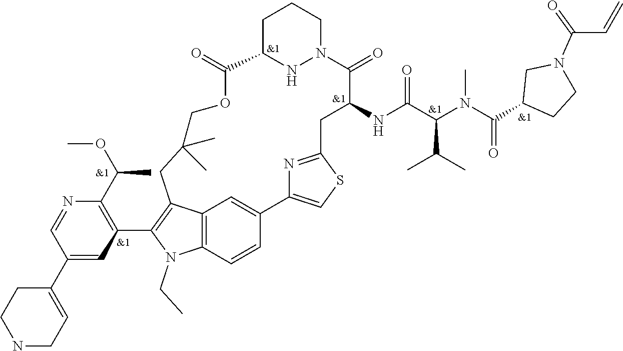 |
| BB328 | 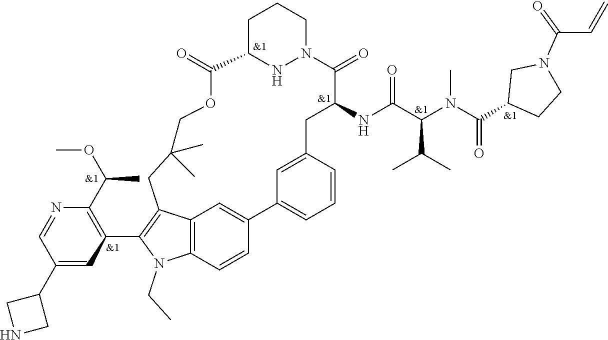 |
| BB329 | 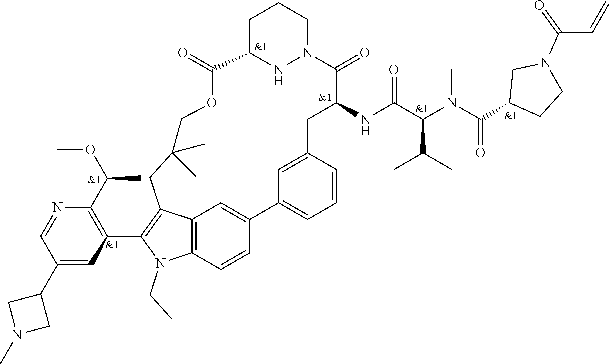 |
| BB330 | 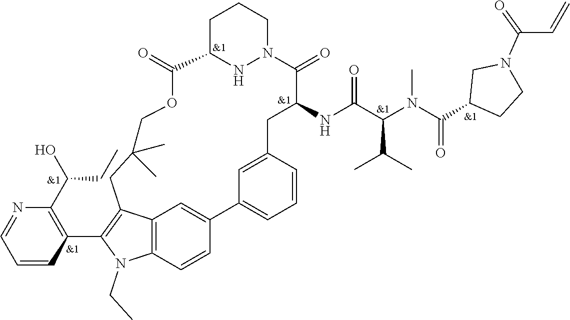 |
| BB331 | 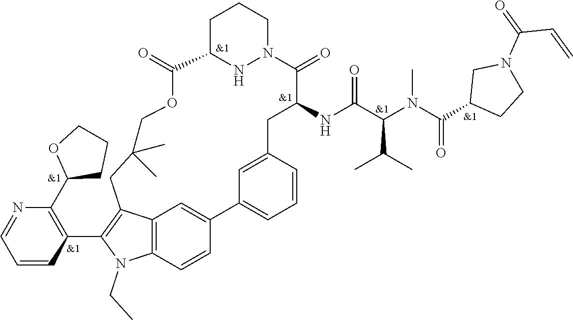 |
| BB332 | 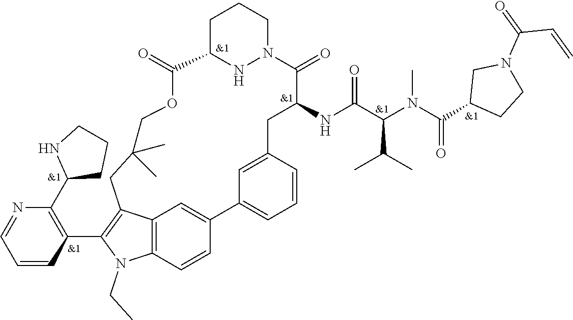 |
| BB333 | 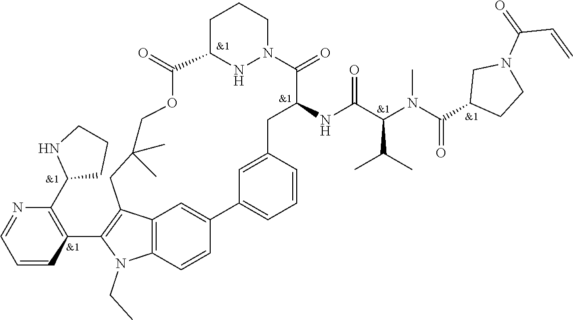 |
| BB334 | 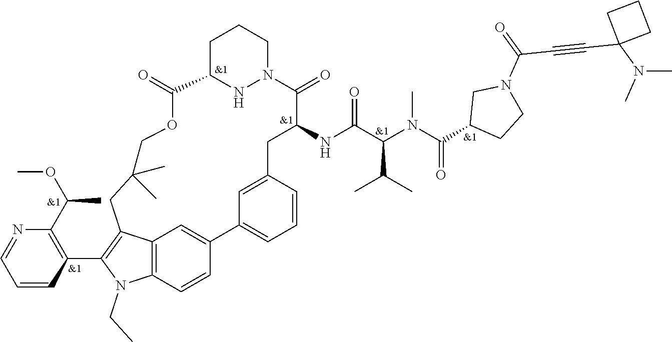 |
| BB335 | 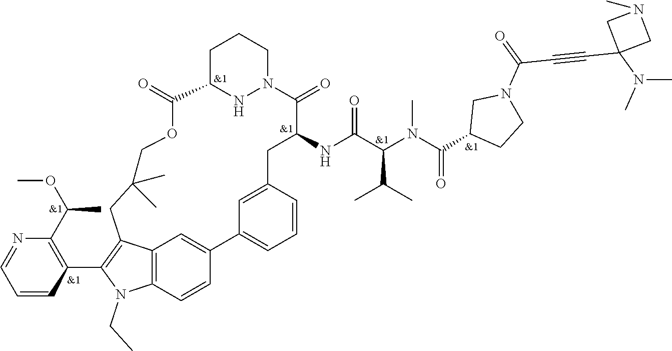 |
| BB336 | 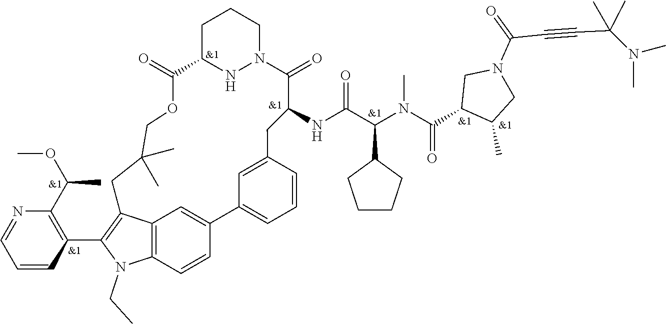 |
| BB337 | 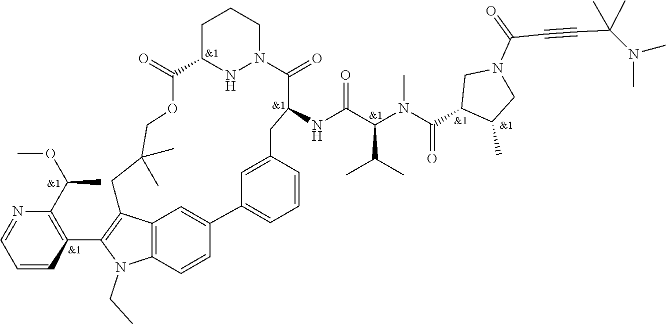 |
| BB338 | 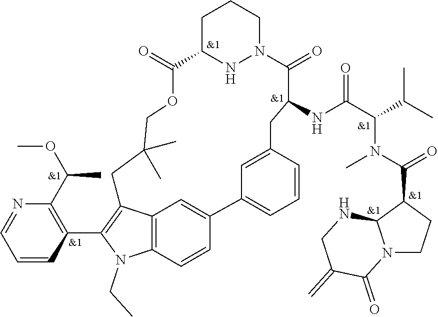 |
| BB339 | 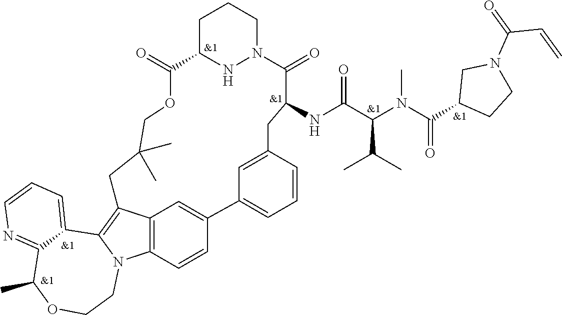 |
| BB340 | 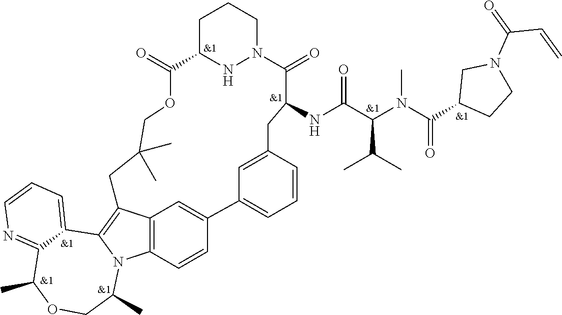 |
| BB341 | 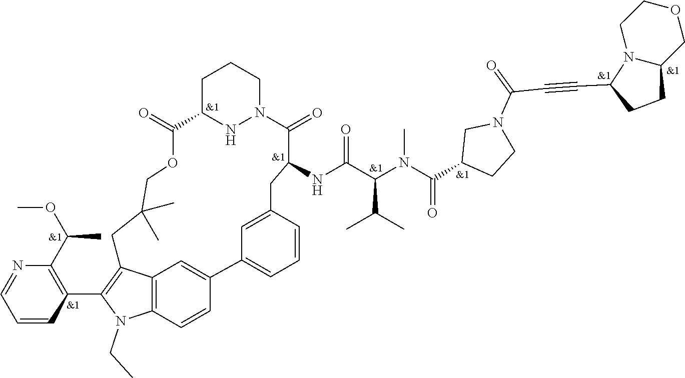 |
| BB342 | 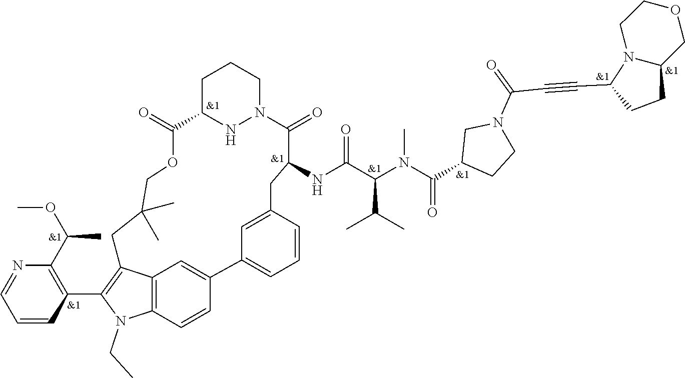 |
| BB343 | 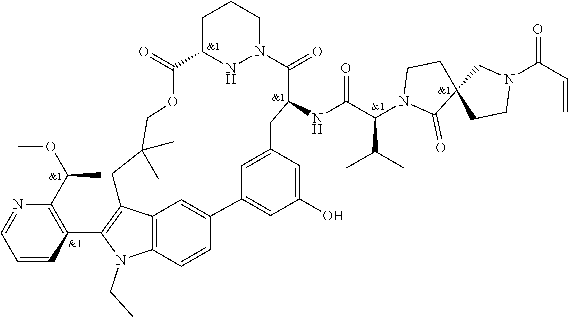 |
| BB344 | 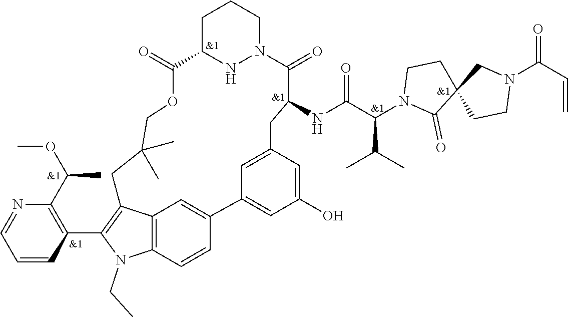 |
| BB345 | 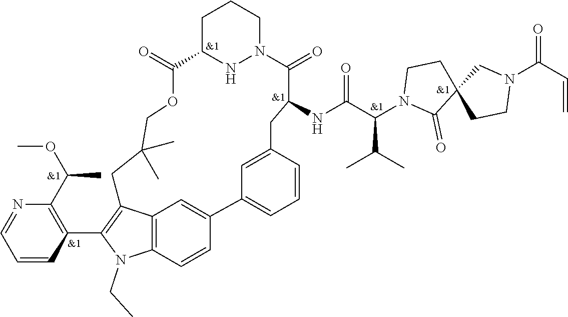 |
| BB346 | 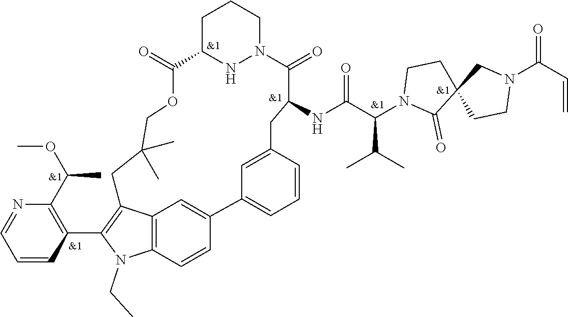 |
| BB347 | 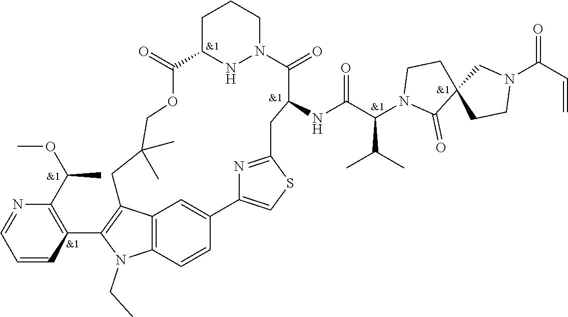 |
| BB348 | 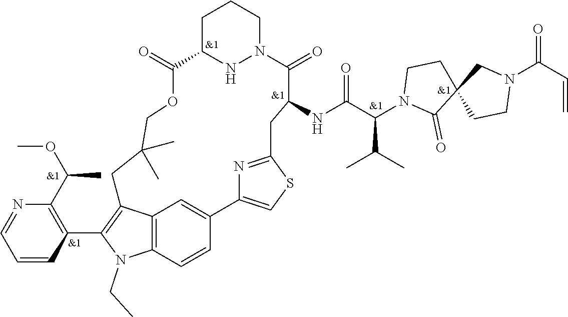 |
| BB349 | 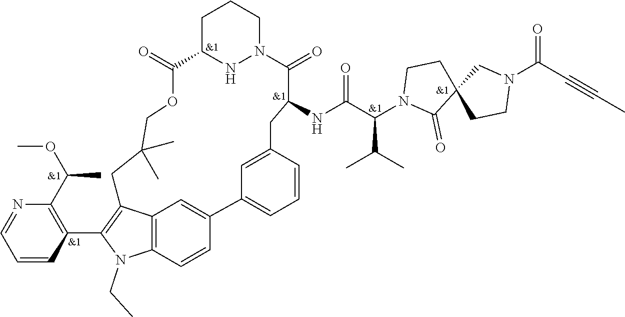 |
| BB350 | 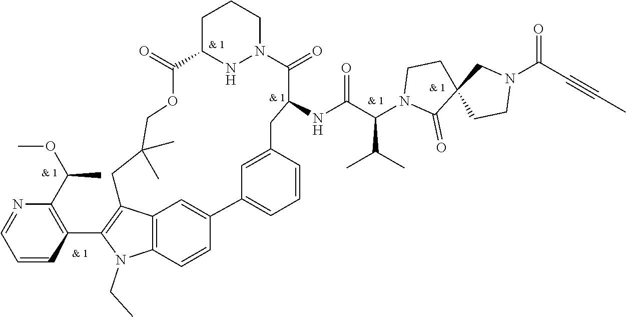 |
| BB351 | 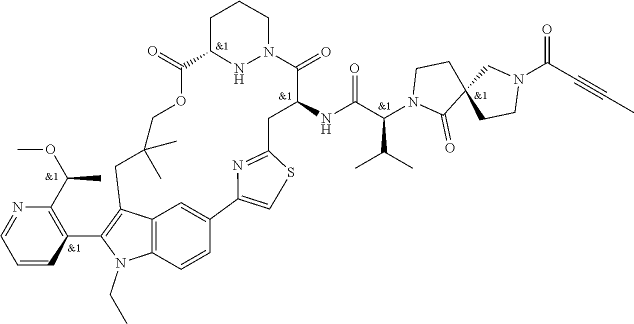 |
| BB352 | 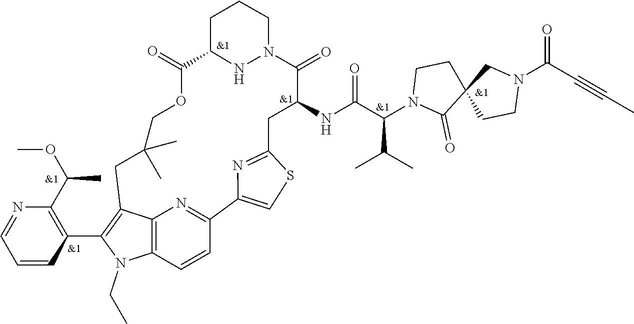 |
| BB353 | 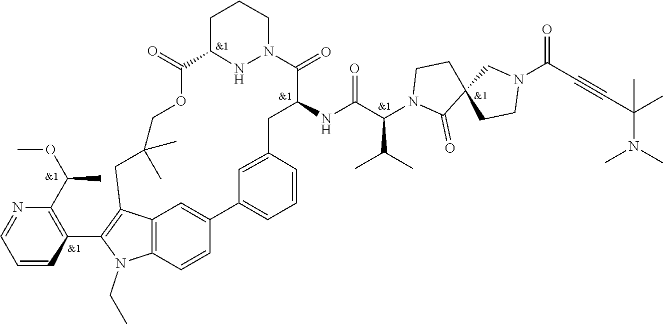 |
| BB354 | 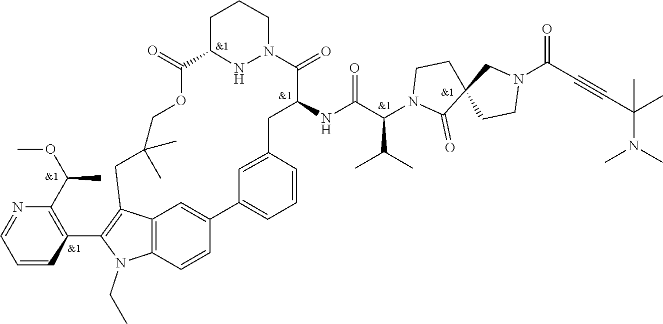 |
| BB355 | 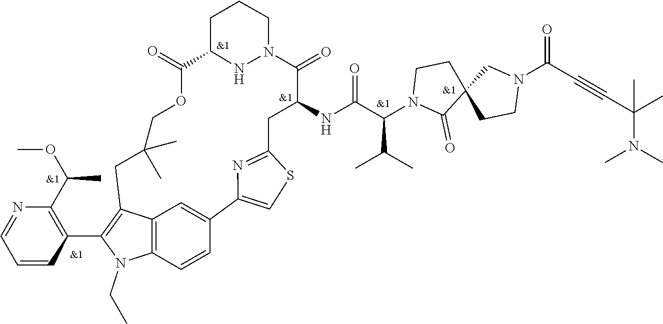 |
| BB356 | 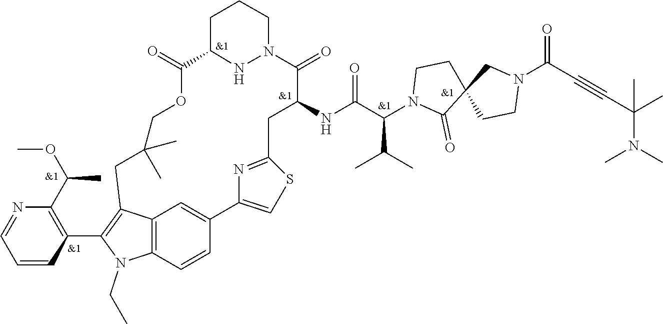 |
| BB357 | 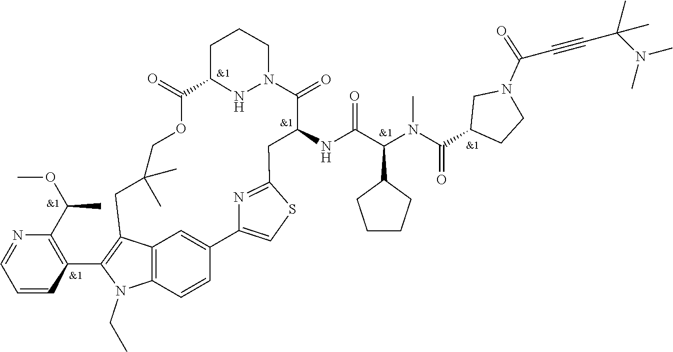 |
| BB358 | 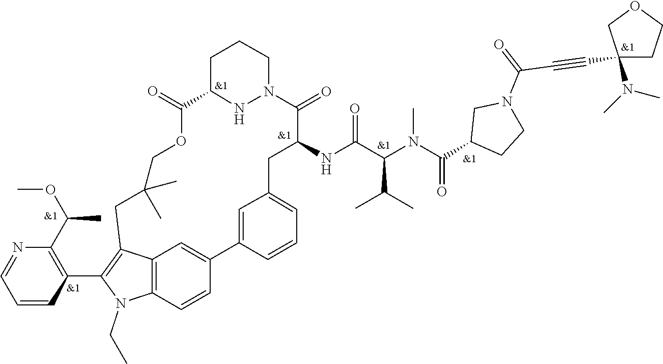 |
| BB359 | 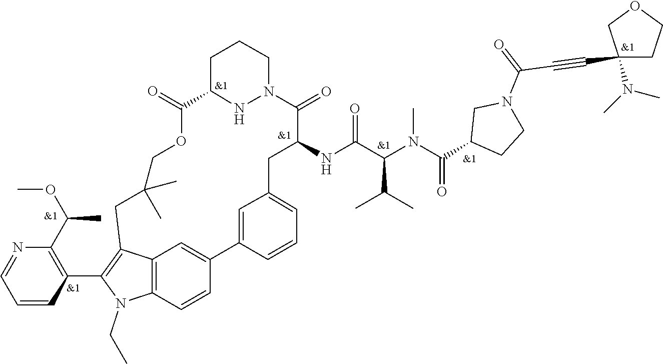 |
| BB360 | 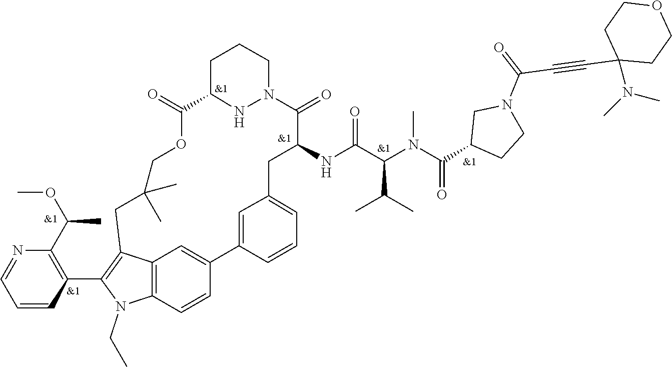 |
| BB361 | 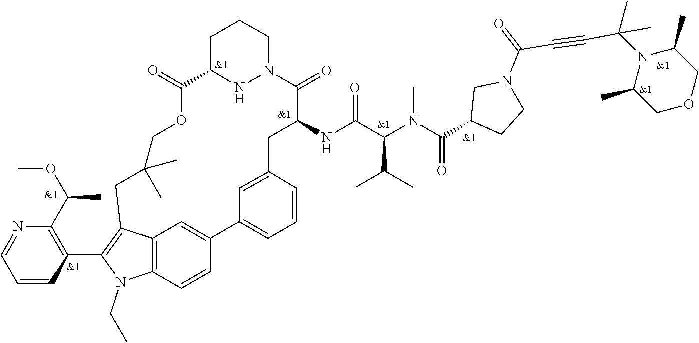 |
| BB362 | 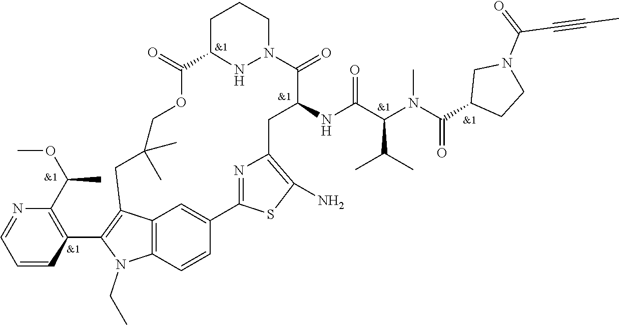 |
| BB363 | 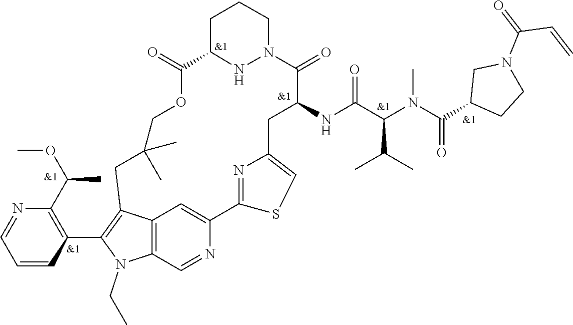 |
| BB364 | 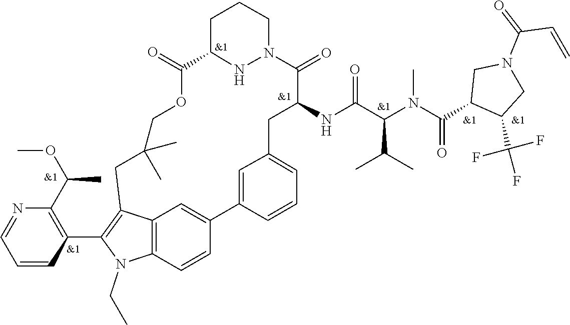 |
| BB365 | 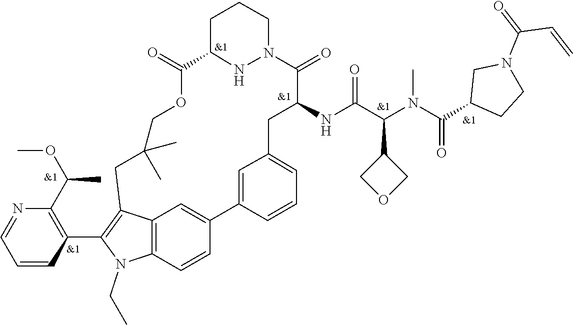 |
| BB366 | 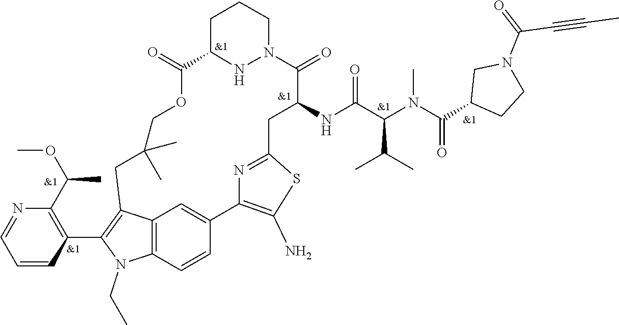 |
| BB367 | 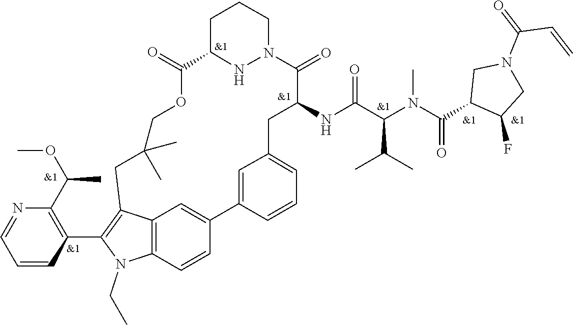 |
| BB368 | 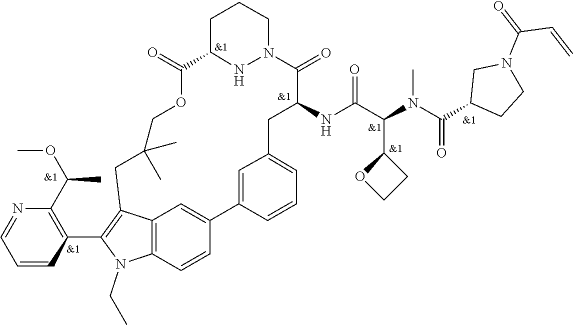 |
| BB369 | 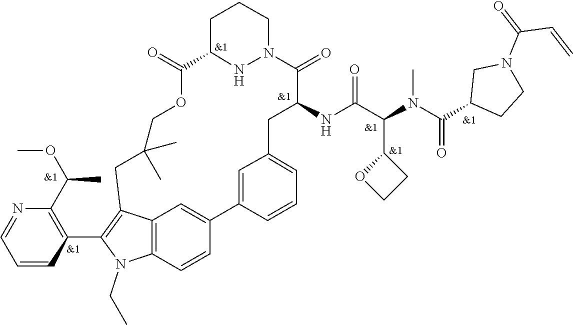 |
| BB370 | 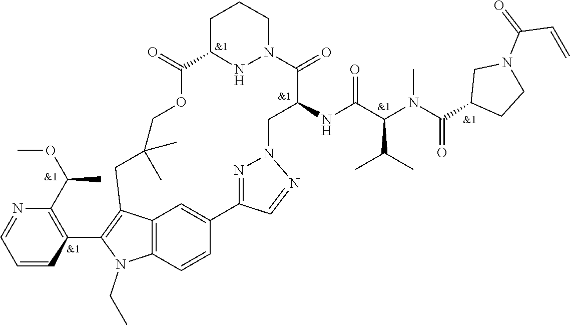 |
| BB371 | 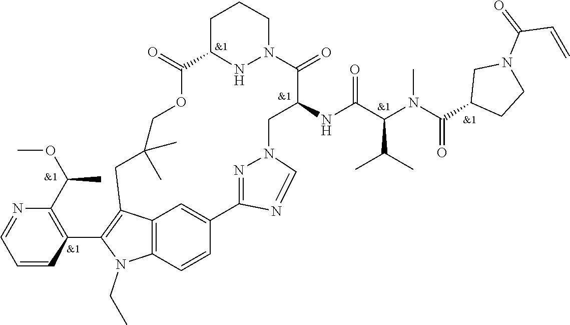 |
| BB372 | 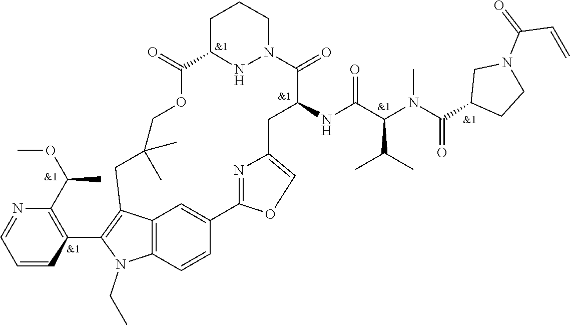 |
| BB373 | 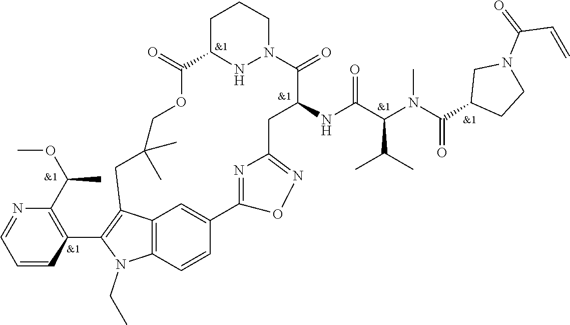 |
| BB374 | 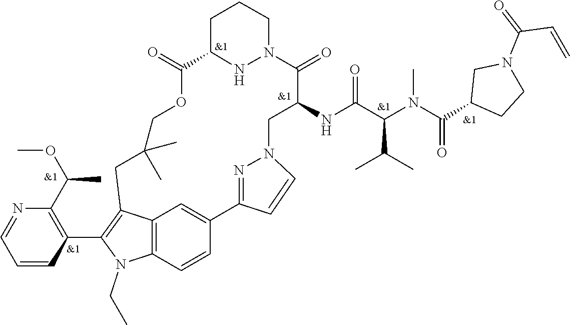 |
| BB375 | 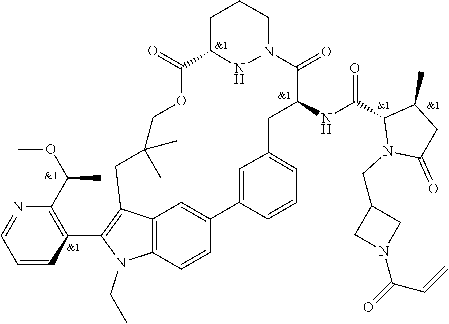 |
| BB376 | 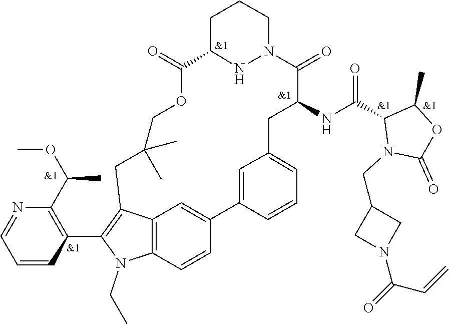 |
| BB377 | 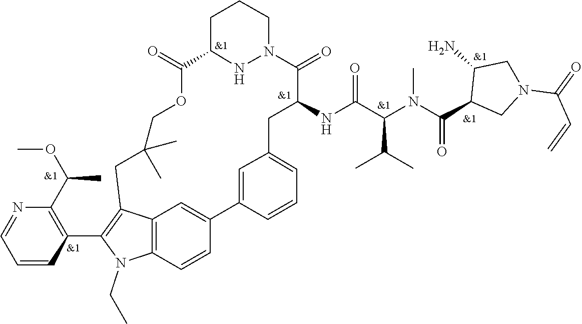 |
| BB378 | 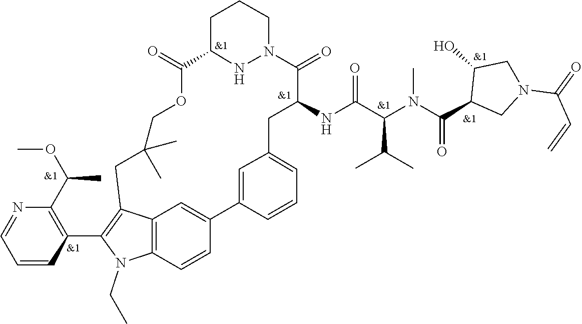 |
| BB379 | 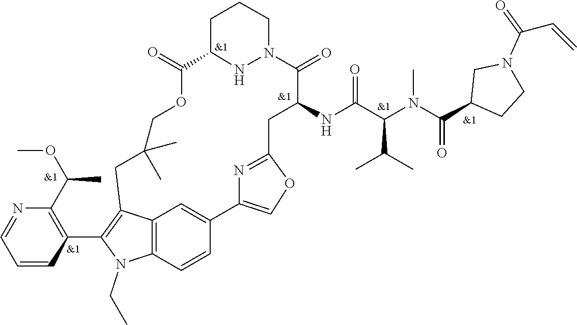 |
| BB380 | 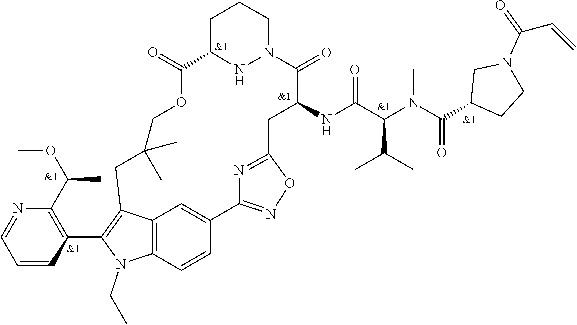 |
| BB381 | 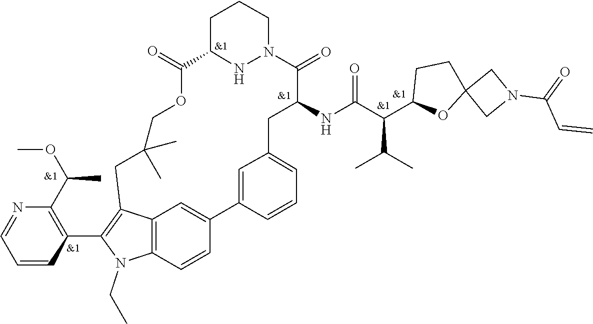 |
| BB382 | 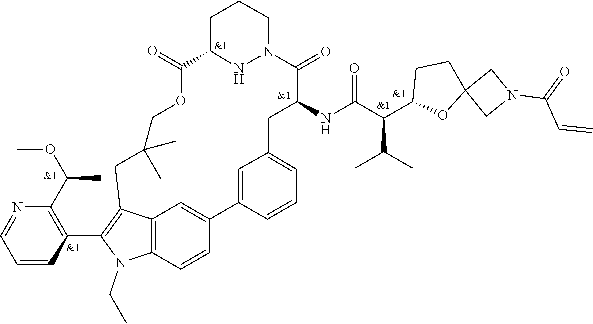 |
| BB383 | 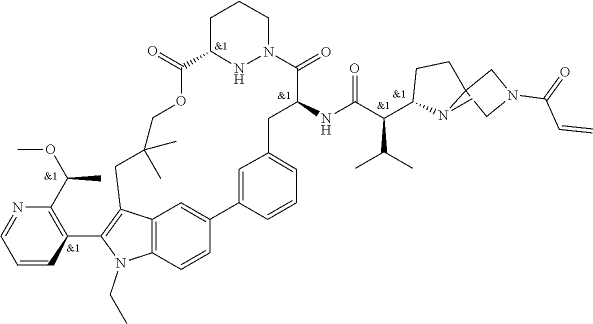 |
| BB384 | 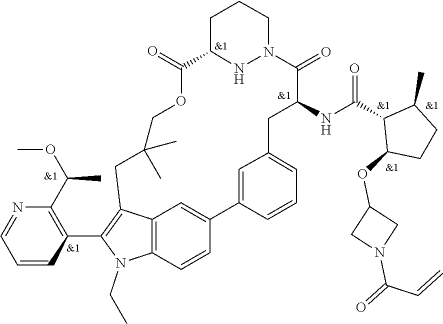 |
| BB385 | 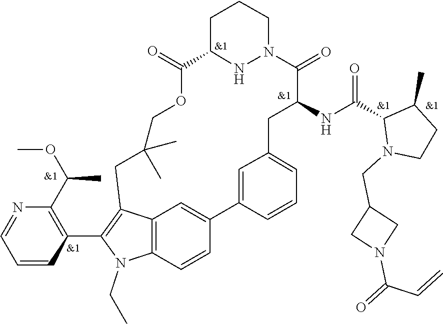 |
| BB386 | 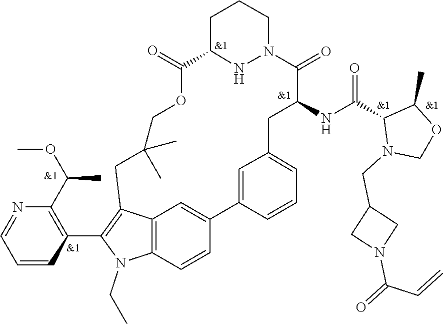 |
| BB387 | 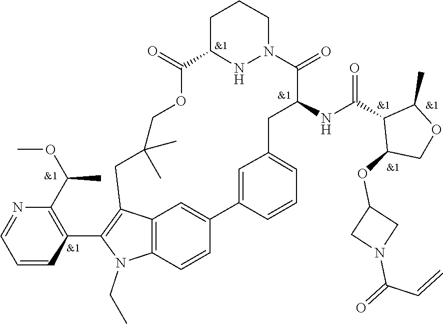 |
| BB388 | 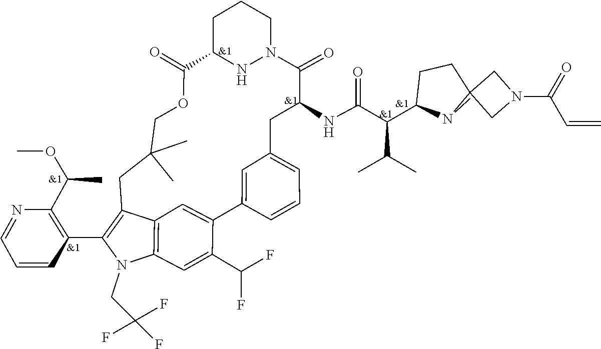 |
| BB389 | 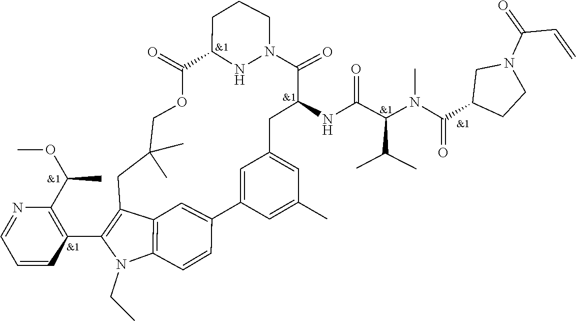 |
| BB390 | 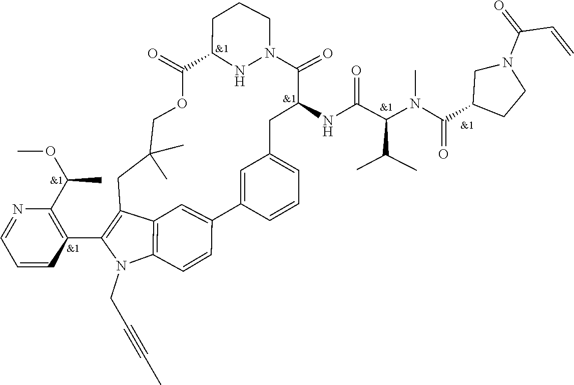 |
| BB391 | 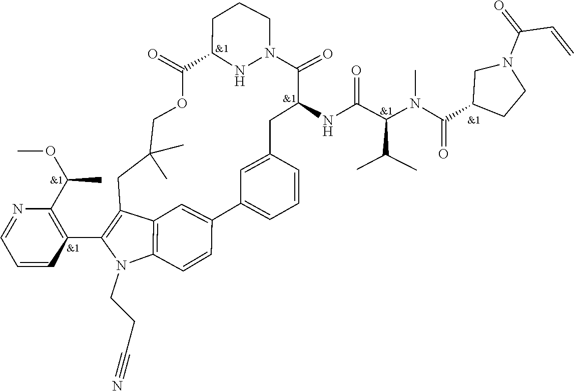 |
| BB392 | 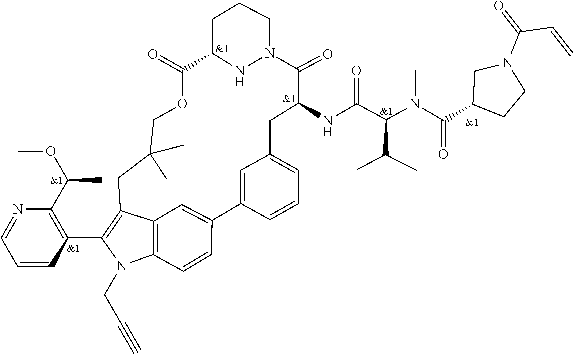 |
| BB393 | 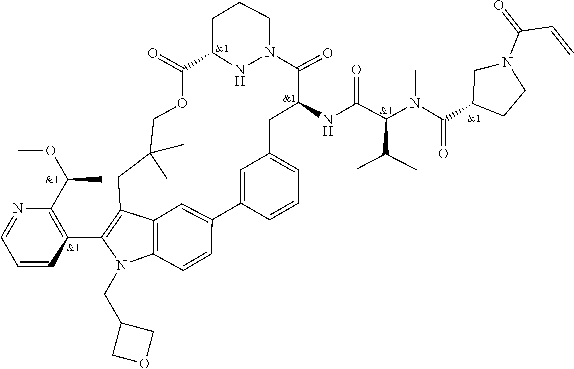 |
| BB394 | 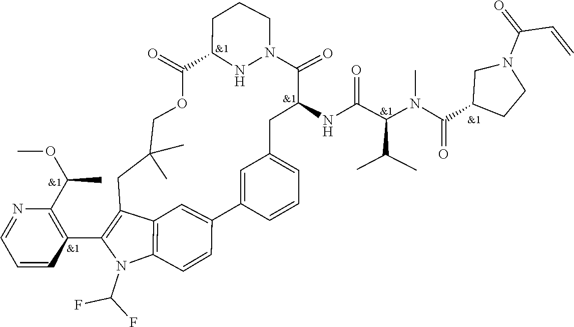 |
| BB395 | 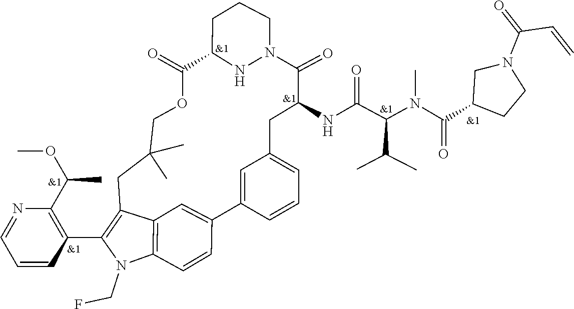 |
| BB396 | 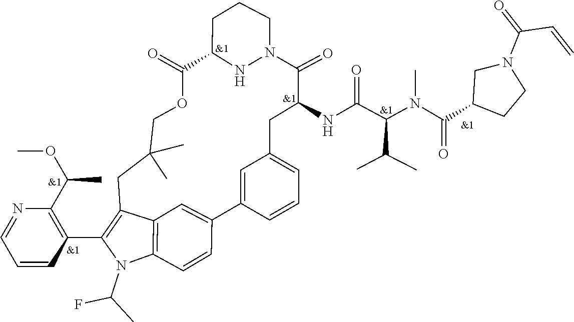 |
| BB397 | 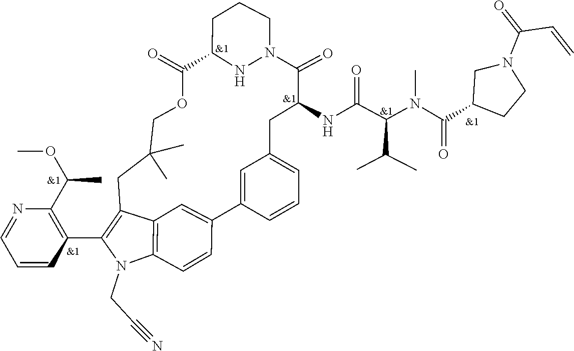 |
| BB398 | 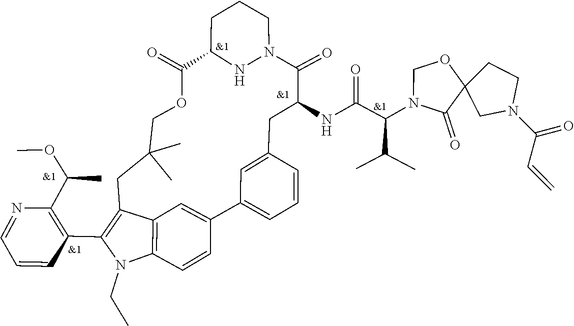 |
| BB399 | 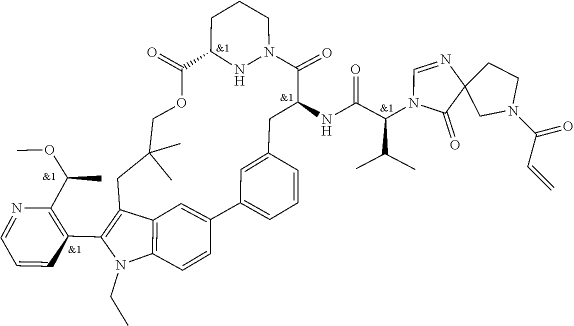 |
| BB400 | 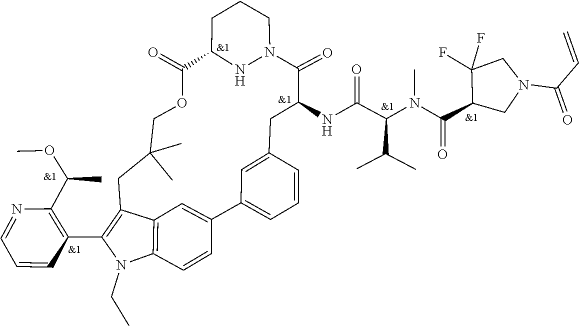 |
| BB401 | 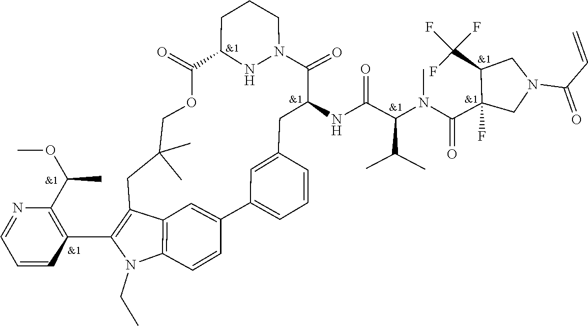 |
| BB402 | 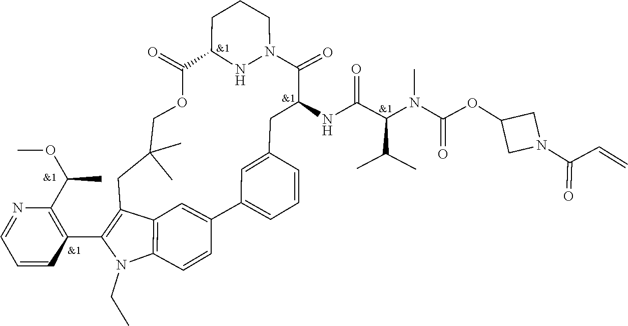 |
| BB403 | 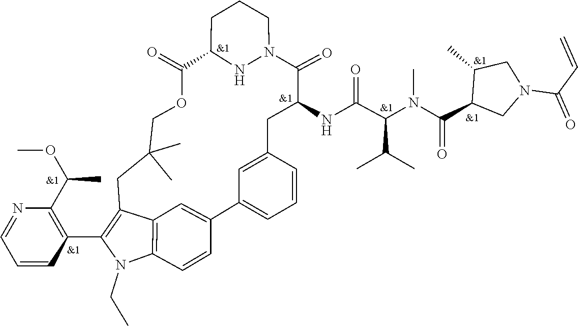 |
| BB404 | 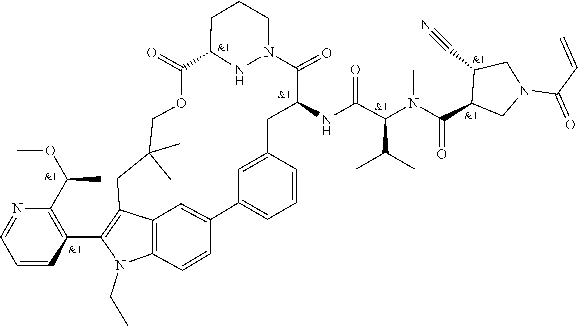 |
| BB405 | 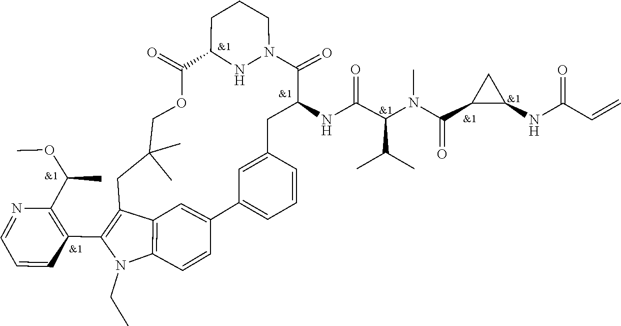 |
| BB406 | 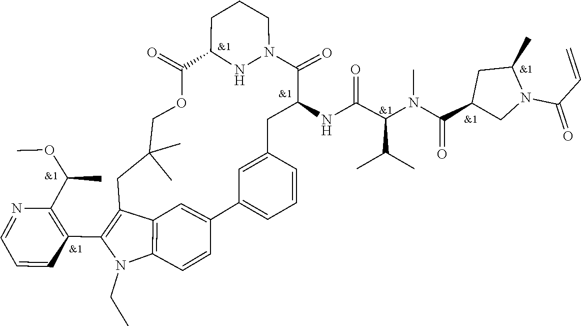 |
| BB407 | 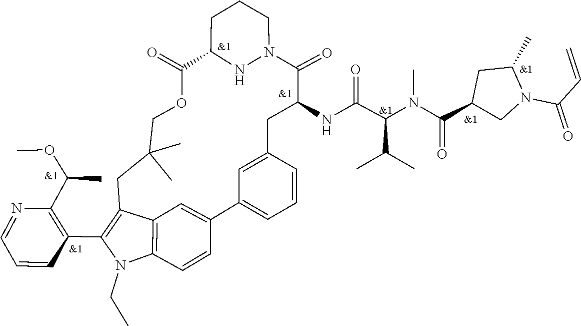 |
| BB408 | 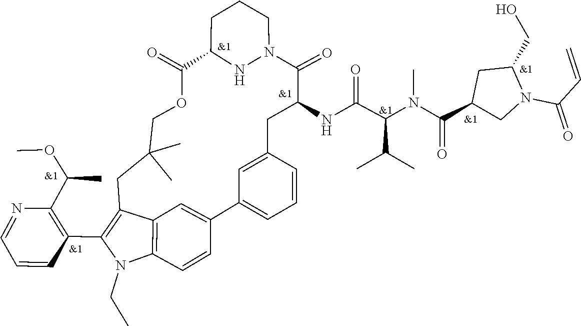 |
| BB409 | 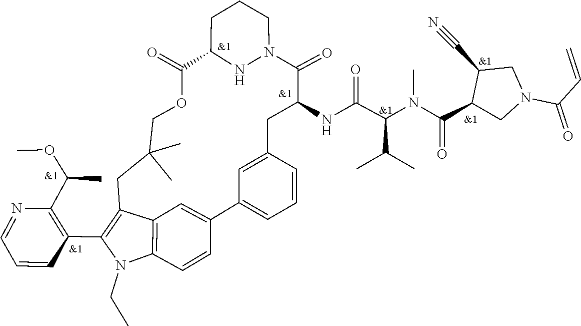 |
| BB410 | 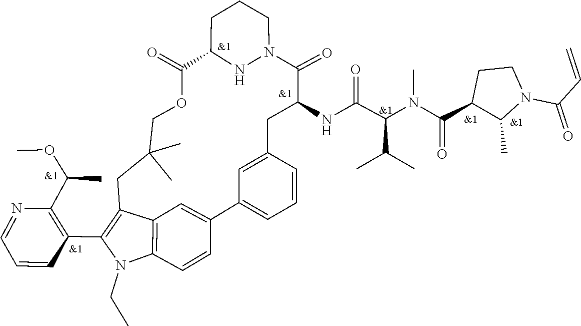 |
| BB411 | 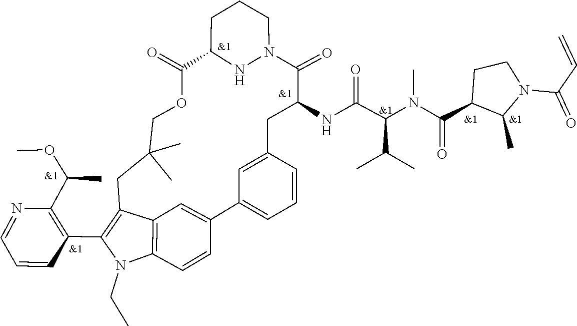 |
| BB412 | 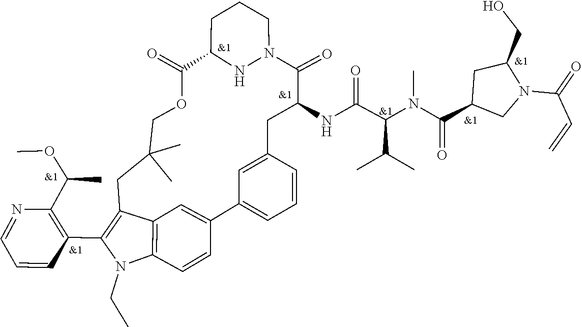 |
| BB413 | 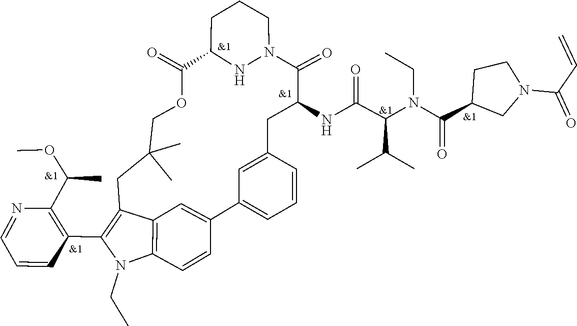 |
| BB414 | 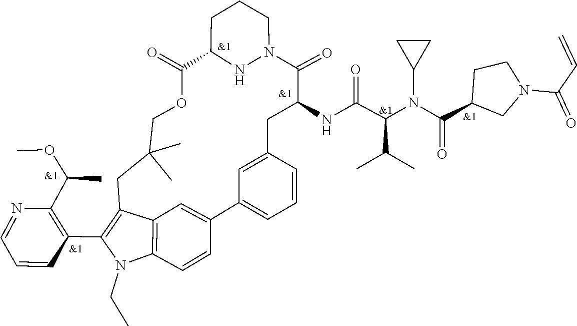 |
| BB415 | 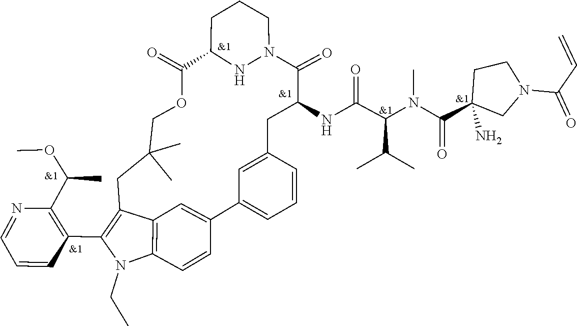 |
| BB416 | 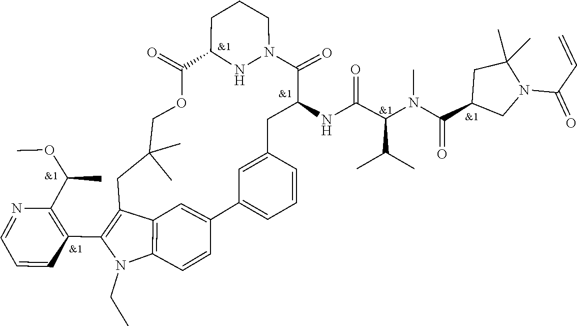 |
| BB417 | 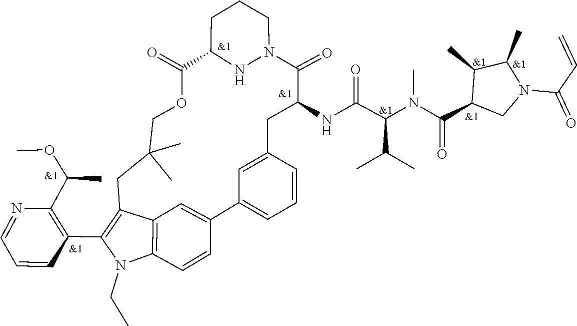 |
| BB418 | 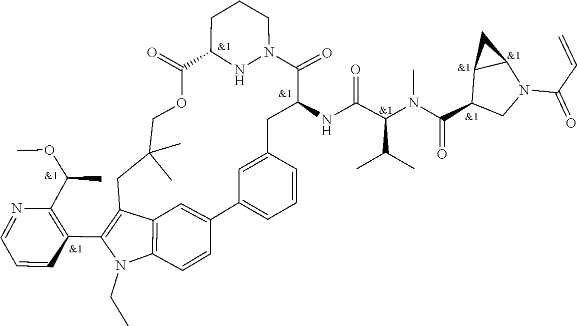 |
| BB419 | 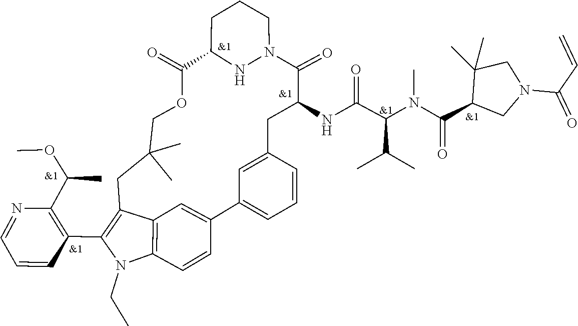 |
| BB420 | 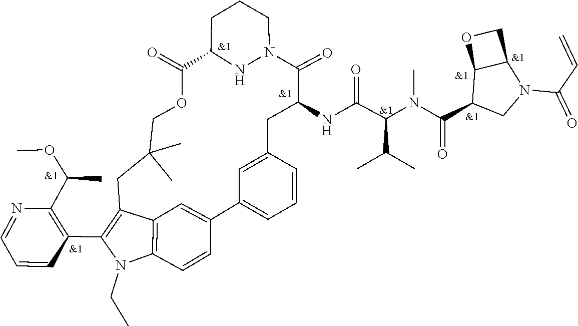 |
| BB421 | 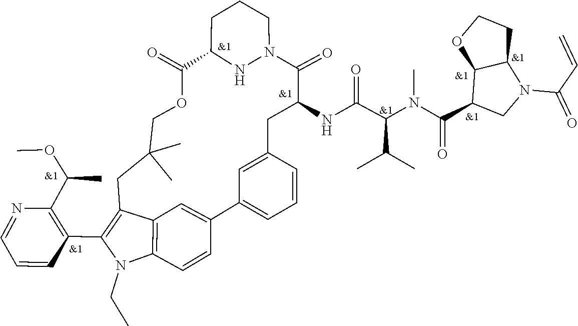 |
| BB422 | 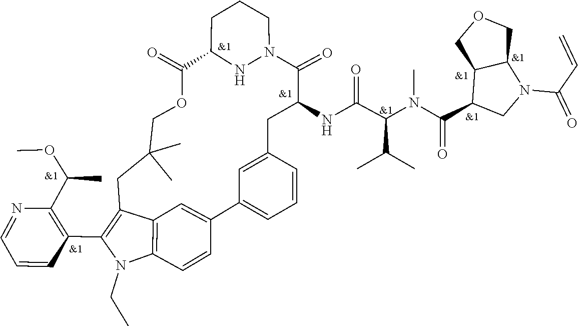 |
| BB423 | 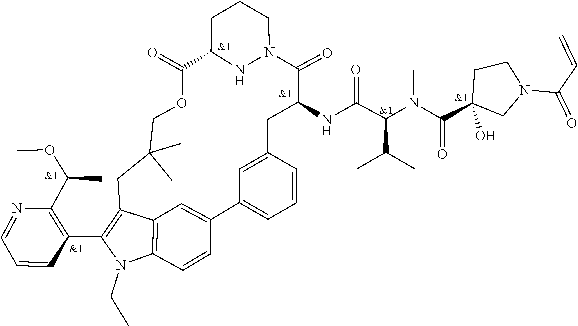 |
| BB424 | 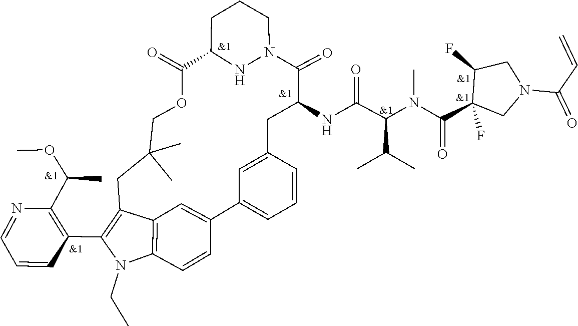 |
| BB425 | 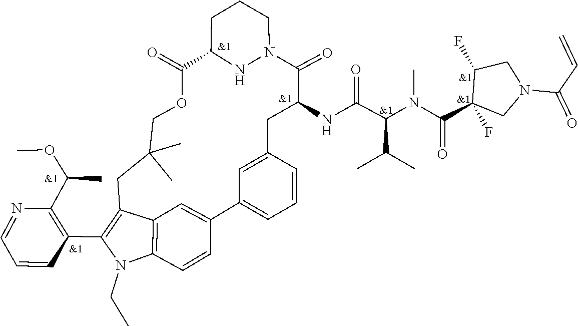 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

In some embodiments, the RAS(ON) inhibitor is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

In some embodiments, the RAS(ON) inhibitor is provided as a conjugate, or salt thereof, comprising the structure of Formula BIV:

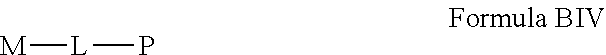

wherein L is a linker;
P is a monovalent organic moiety; and
M has the structure of Formula BVa:

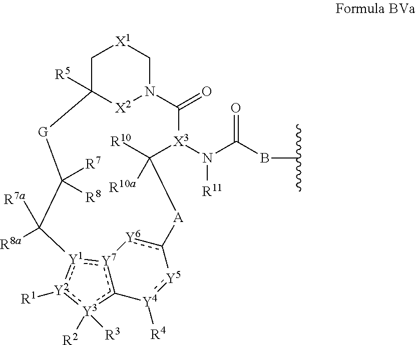

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is absent, —CH(R⁹)—, >C═CR⁹R⁹′, or >CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments the conjugate, or salt thereof, comprises the structure of Formula BIV:

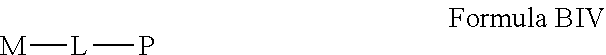

wherein L is a linker;
P is a monovalent organic moiety; and
M has the structure of Formula BVb:

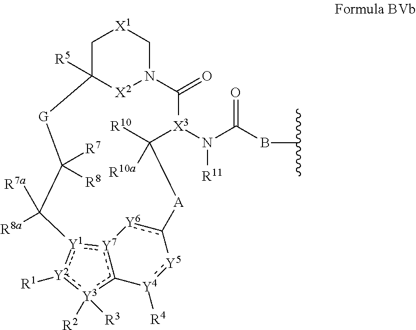

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the conjugate has the structure of Formula BIV:

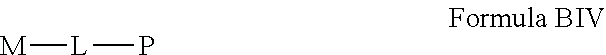

wherein L is a linker;
P is a monovalent organic moiety; and
M has the structure of Formula BVc:

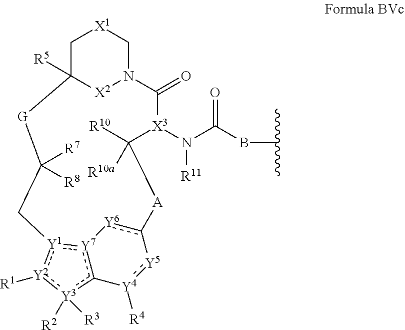

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, the RAS(ON) inhibitor has the structure of Formula BIV:

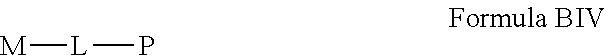

wherein L is a linker;
P is a monovalent organic moiety; and
M has the structure of Formula BVd:

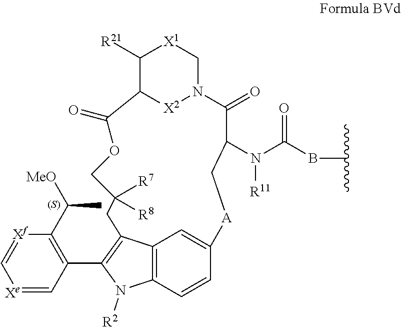

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl, C₁-C₆ fluoroalkyl, or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of formula BI and subformula thereof, X^{e} is N and X^{f} is CH. In some embodiments, X^{e} is CH and X^{f} is N.

In some embodiments, the RAS(ON) inhibitor has the structure of Formula BIV:

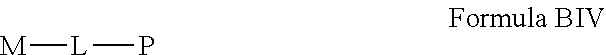

wherein L is a linker;
P is a monovalent organic moiety; and
M has the structure of Formula BVe:

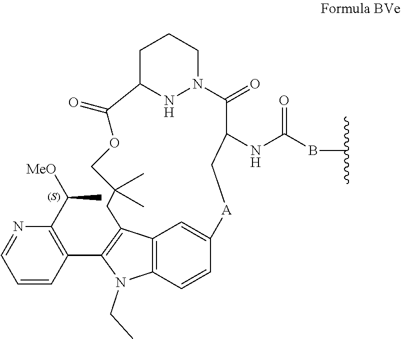

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of a conjugate of Formula BIV, the linker has the structure of Formula BII:

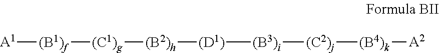

where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C₁ and C₂ are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A².

In some embodiments of a conjugate of formula BIV, the monovalent organic moiety is a protein, such as a Ras protein. In some embodiments, the Ras protein is K-Ras G12C, K-Ras G13C, H-Ras G12C, H-Ras G13C, N-Ras G12C, or N-Ras G13C. Other Ras proteins are described herein. In some embodiments, the linker is bound to the monovalent organic moiety through a bond to a sulfhydryl group of an amino acid residue of the monovalent organic moiety. In some embodiments, the linker is bound to the monovalent organic moiety through a bond to a carboxyl group of an amino acid residue of the monovalent organic moiety.

The compounds described in Tables B1 and B2 may be made from commercially available starting materials or synthesized using known organic, inorganic, or enzymatic processes. The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below or as described in WO 2021/091982.

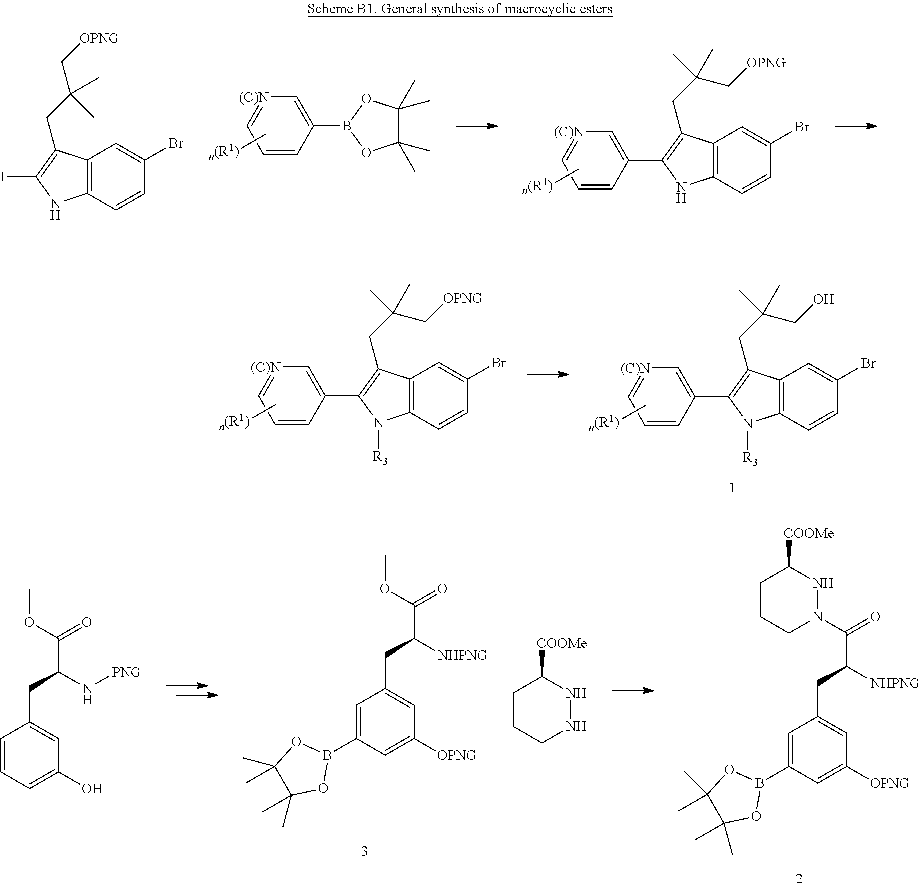

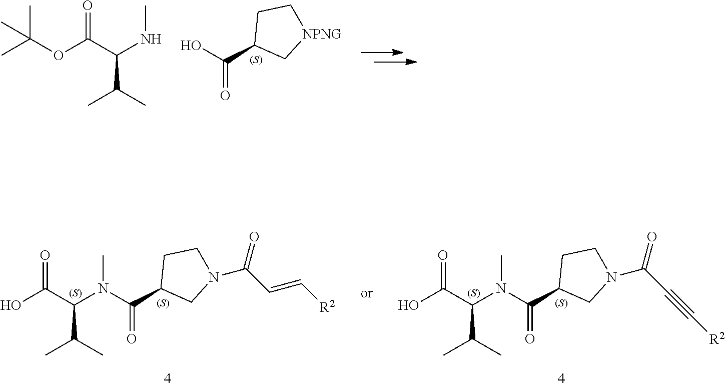

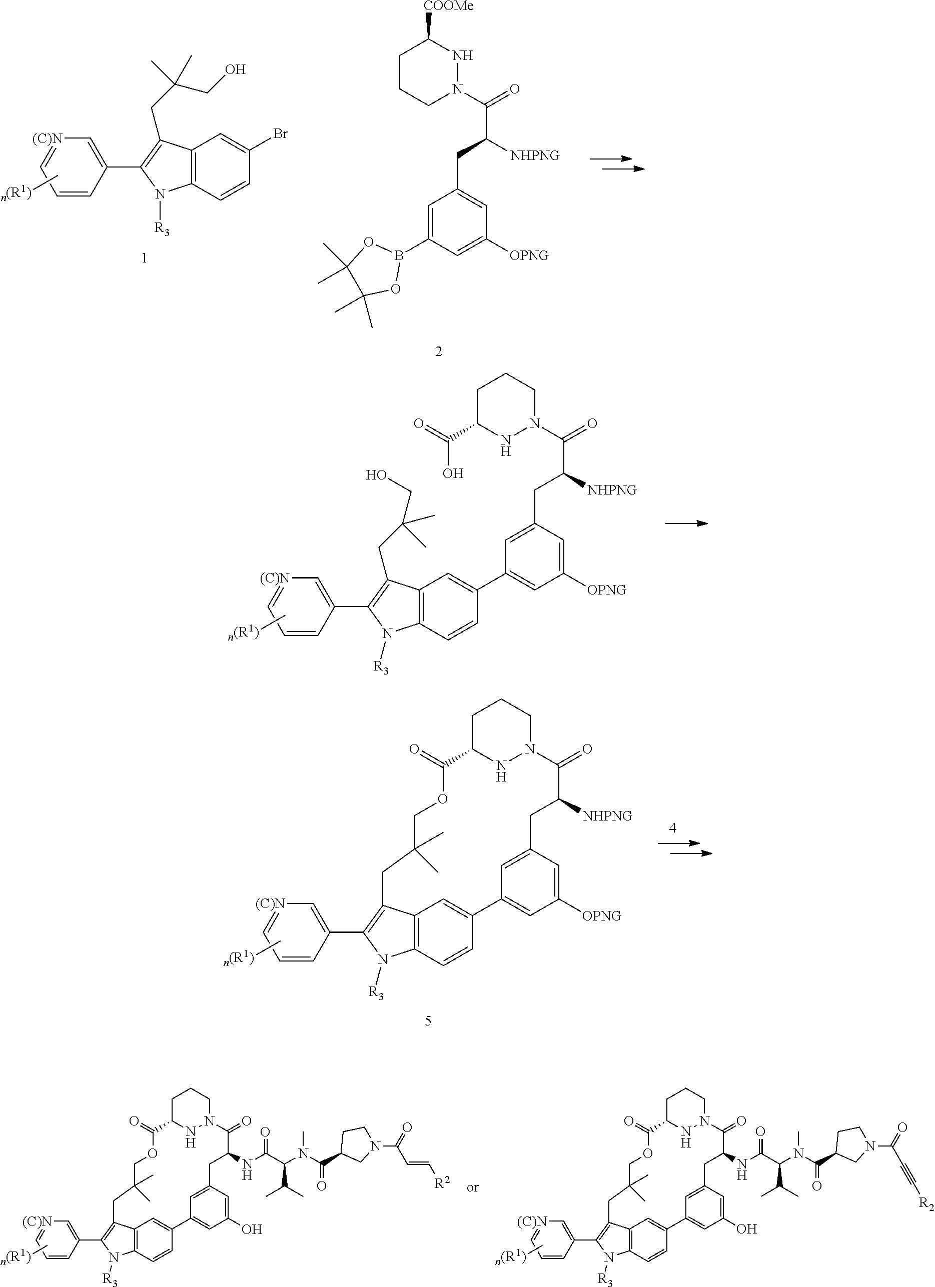

A general synthesis of macrocyclic esters is outlined in Scheme B1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions. Methyl-amino-hexahydropyridazine-3-carboxylate-boronic ester (2) can be prepared in three steps, including protection, iridium catalyst mediated borylation, and coupling with methyl (S)-hexahydropyridazine-3-carboxylate.

An appropriately substituted acetylpyrrolidine-3-carbonyl-N-methyl-L-valine (or an alternative aminoacid derivative (4) can be made by coupling of methyl-L-valinate and protected (S)-pyrrolidine-3-carboxylic acid, followed by deprotection, coupling with a carboxylic acid containing an appropriately substituted Michael acceptor, and a hydrolysis step.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of a Pd catalyst followed by hydrolysis and macrolactonization steps to result in an appropriately protected macrocyclic intermediate (5). Deprotection and coupling with an appropriately substituted intermediate 4 results in a macrocyclic product. Additional deprotection and/or functionalization steps can be required to produce the final compound.

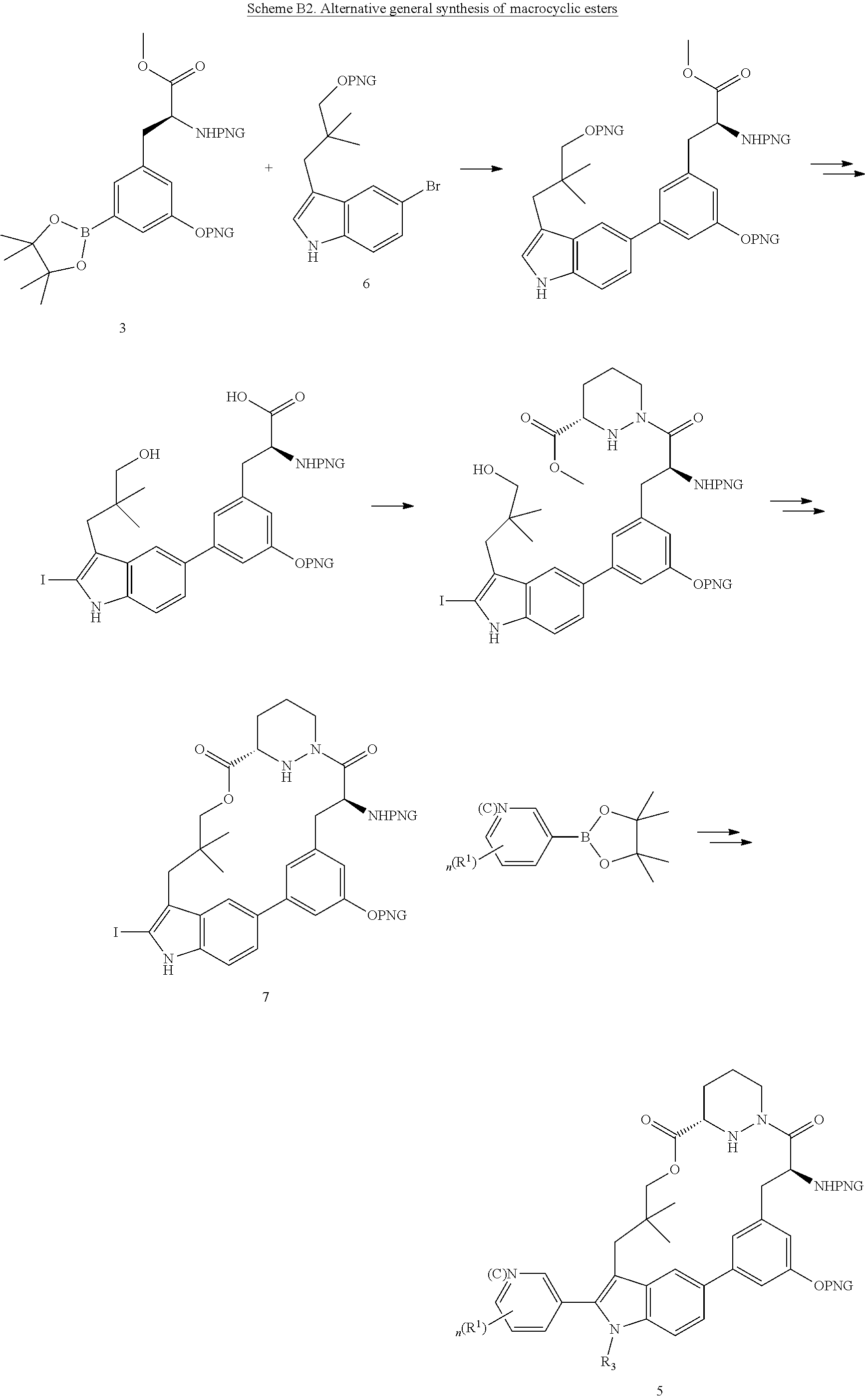

Alternatively, macrocyclic ester can be prepared as described in Scheme B2. An appropriately protected bromo-indolyl (6) coupled in the presence of a Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of a Pd catalyst with an appropriately substituted boronic ester and alkylation can yield fully protected macrocycle (5). Additional deprotection or functionalization steps are required to produce the final compound.

In addition, compounds of the disclosure can be synthesized using the methods described in the Examples below or as described in WO 2021/091982, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (BI), where B, L and Ware defined herein, including by using methods exemplified in the Example section herein and in WO 2021/091982.

Compounds of Table B1 herein were prepared using methods disclosed herein or were prepared using methods disclosed herein combined with the knowledge of one of skill in the art. Compounds of Table B2 may be prepared using methods disclosed herein or may be prepared using methods disclosed herein combined with the knowledge of one of skill in the art.

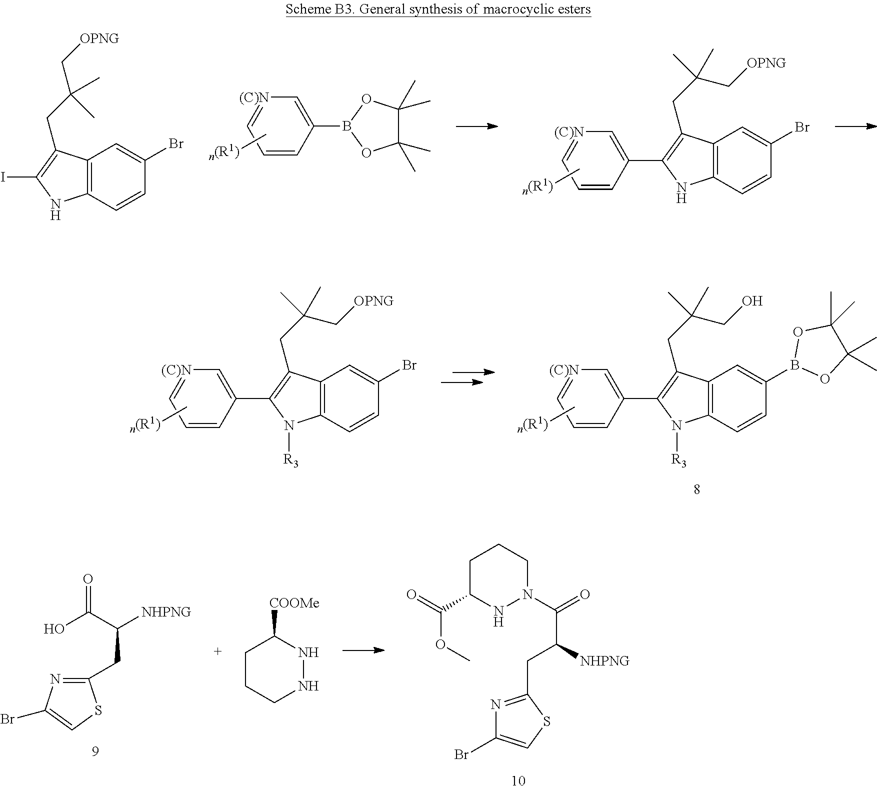

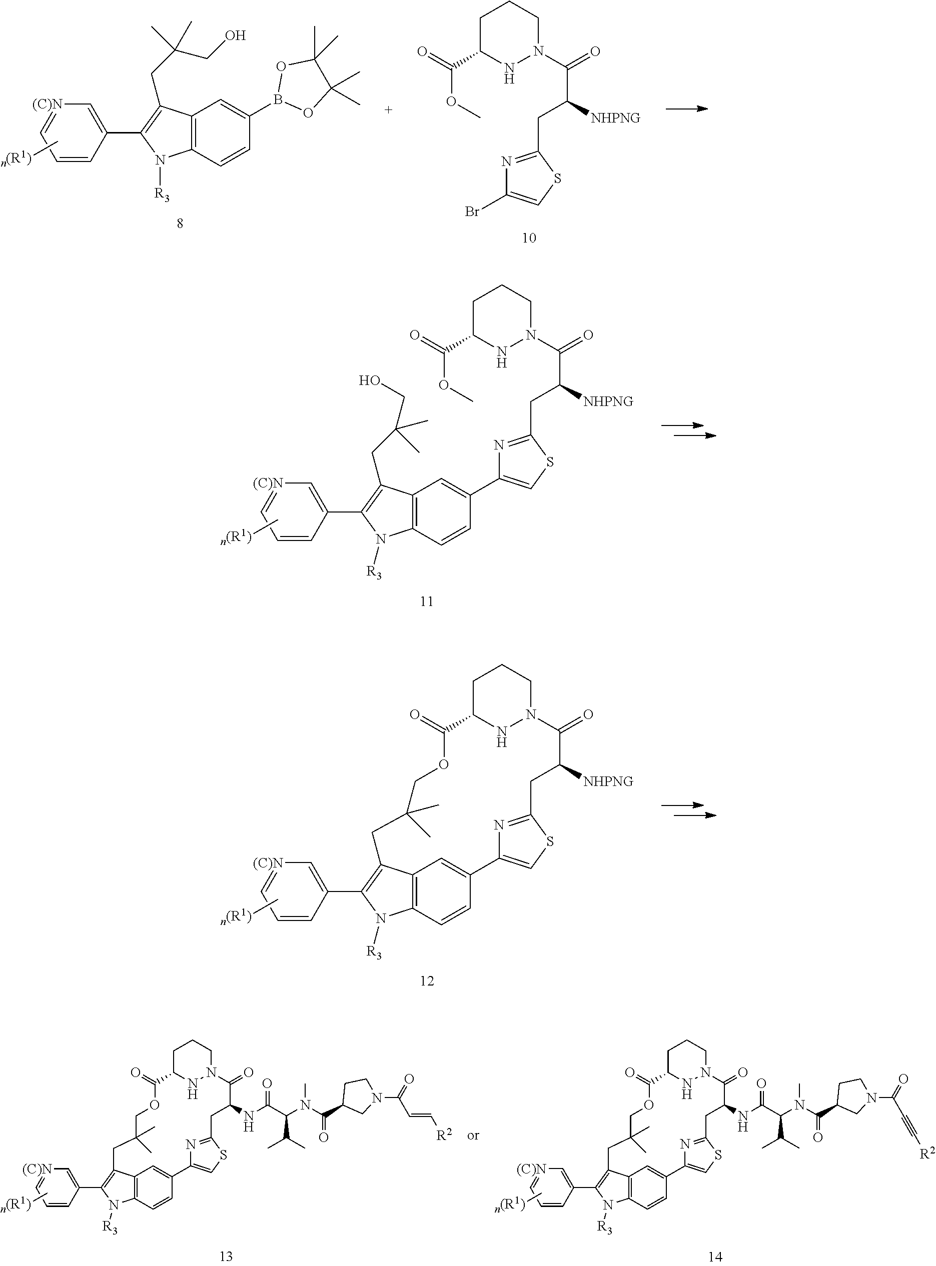

An alternative general synthesis of macrocyclic esters is outlined in Scheme B3. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macrolactonization steps to result in an appropriately protected macrocyclic intermediate (11). Deprotection and coupling with an appropriately substituted intermediate 4 can result in a macrocyclic product. Additional deprotection or functionalization steps could be required to produce a final compound 13 or 14.

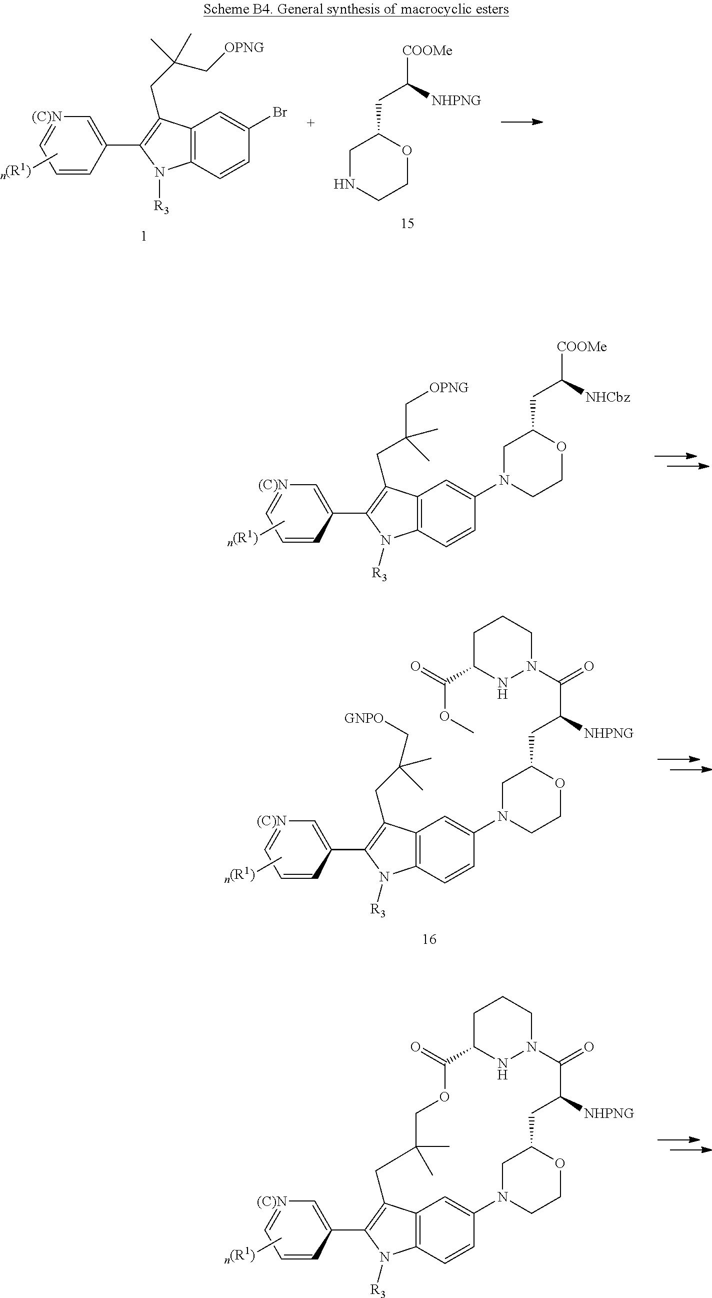

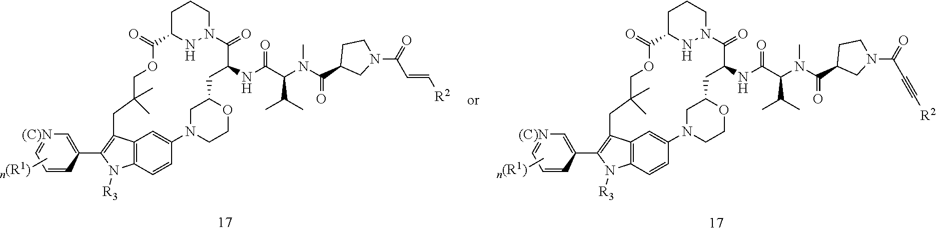

An alternative general synthesis of macrocyclic esters is outlined in Scheme B4. An appropriately substituted morpholine or an alternative heterocyclic intermediate (15) can be coupled with appropriately protected Intermediate 1 via Palladium mediated coupling. Subsequent ester hydrolysis, and coupling with piperazoic ester results in intermediate 16.

The macrocyclic esters can be made by hydrolysis, deprotection and macrocyclization sequence. Subsequent deprotection and coupling with Intermediate 4 (or analogs) result in an appropriately substituted final macrocyclic products. Additional deprotection or functionalization steps could be required to produce a final compound 17.

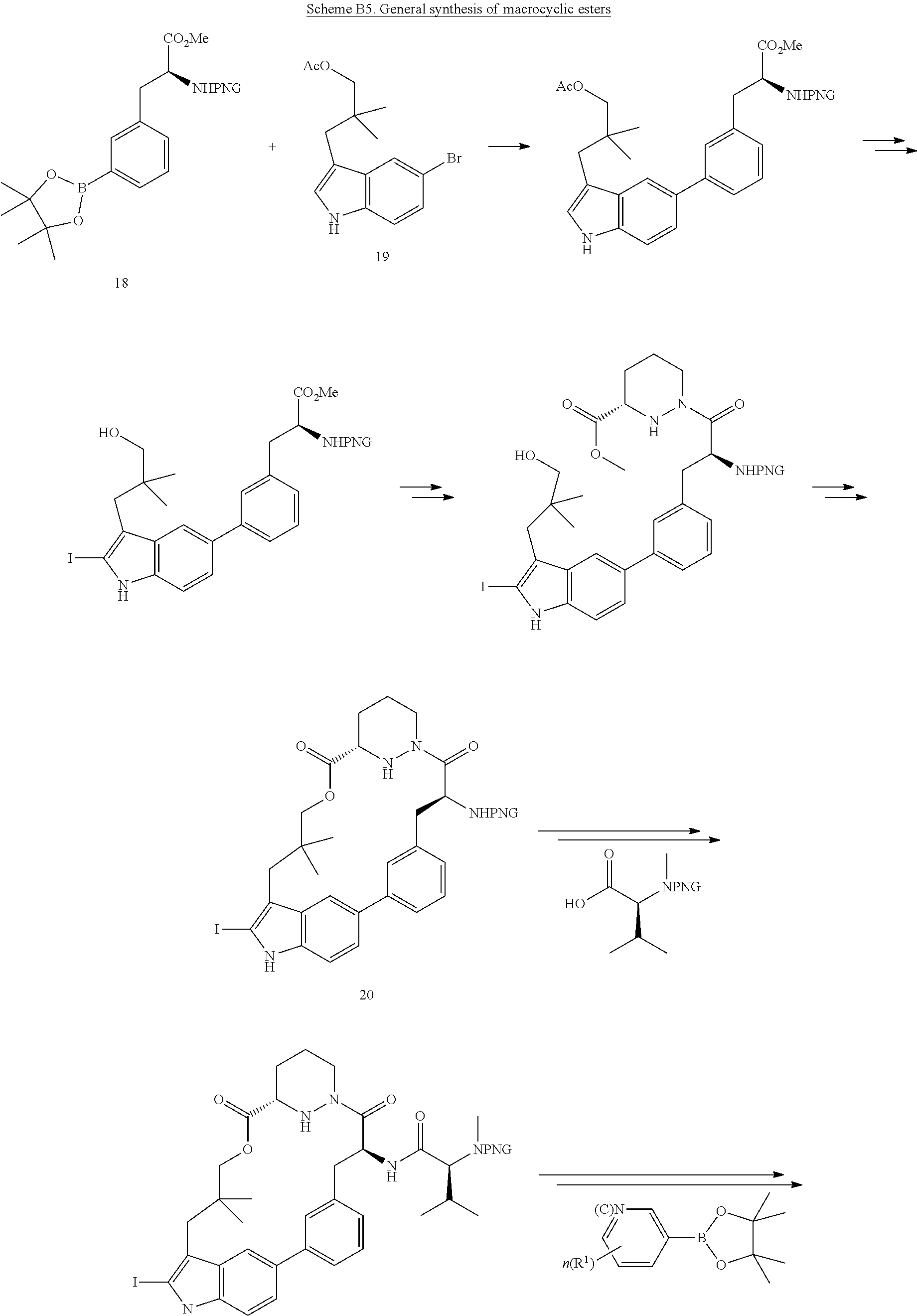

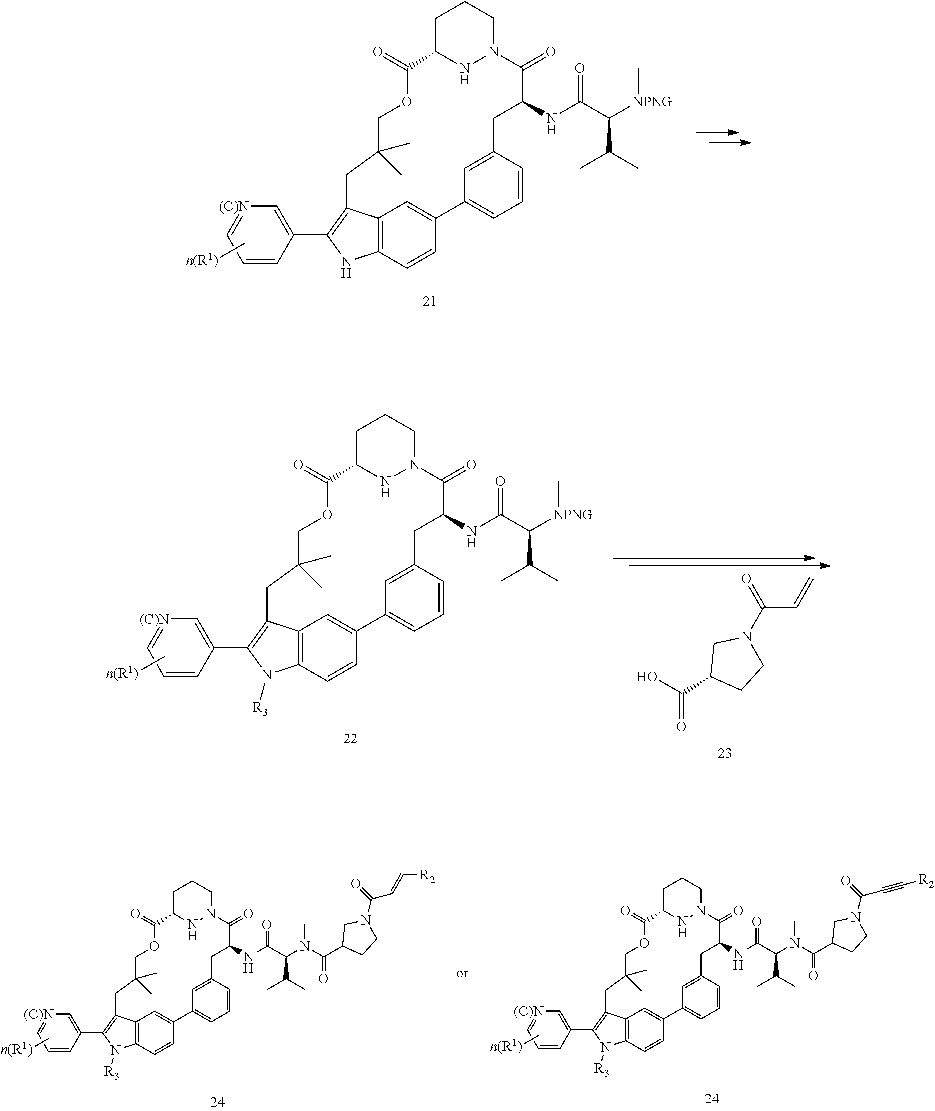

An alternative general synthesis of macrocyclic esters is outlined in Scheme B5. An appropriately substituted macrocycle (20) can be prepared starting from an appropriately protected boronic ester 18 and bromo indolyl intermediate (19), including Palladium mediated coupling, hydrolysis, coupling with piperazoic ester, hydrolysis, de-protection, and macrocyclizarion steps. Subsequent coupling with an appropriately substituted protected amino acid followed by palladium mediated coupling yiels intermediate 21. Additional deprotection and derivatization steps, including alkylation may be required at this point.

The final macrocyclic esters can be made by coupling of intermediate (22) and an appropriately substituted carboxylic acid intermediate (23). Additional deprotection or functionalization steps could be required to produce a final compound (24).

In addition, compounds of the disclosure can be synthesized using the methods described in the Examples below and in WO 2021/091982, together with synthetic methods known in the art of synthetic organic chemistry, or variations thereon as appreciated by those skilled in the art. These methods include but are not limited to those methods described in the Examples below. For example, a person of skill in the art would be able to install into a macrocyclic ester a desired —B-L-W group of a compound of Formula (BI), where B, L and Ware defined herein, including by using methods exemplified in the WO 2021/091982.

In some embodiments, the RAS(ON) inhibitor is a compound, or a pharmaceutically acceptable salt thereof, having the structure of Formula CI:

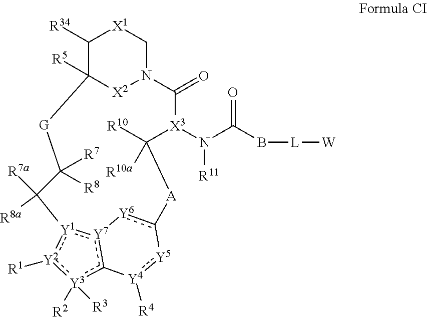

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl; and
R³⁴ is hydrogen or C₁-C₃ alkyl (e.g., methyl).

In some embodiments of Formula CI and subformula thereof, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula CI and subformula thereof, R³⁴ is hydrogen.

In some embodiments of Formula CI and subformula thereof, G is optionally substituted C₁-C₄ heteroalkylene.

In some embodiments, the RAS(ON) inhibitor has the structure of Formula CIa, or a pharmaceutically acceptable salt thereof:

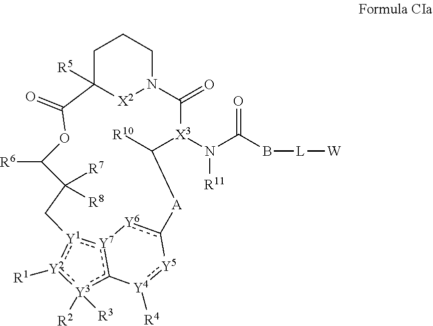

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments of Formula CI and subformula thereof, X² is NH. In some embodiments, X³ is CH.

In some embodiments of Formula CI and subformula thereof, R¹¹ is hydrogen. In some embodiments, R¹¹ is C₁-C₃ alkyl, such as methyl.

In some embodiments, the RAS(ON) inhibitor has the structure of Formula CIb, or a pharmaceutically acceptable salt thereof:

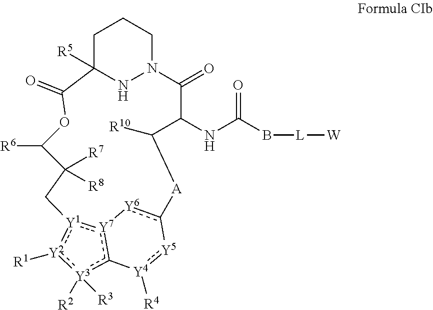

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula CI and subformula thereof, X¹ is optionally substituted C₁-C₂ alkylene. In some embodiments, X¹ is methylene.

In some embodiments of Formula CI and subformula thereof, R⁴ is hydrogen.

In some embodiments of Formula CI and subformula thereof, R⁵ is hydrogen. In some embodiments, R⁵ is C₁-C₄ alkyl optionally substituted with halogen. In some embodiments, R⁵ is methyl.

In some embodiments of Formula CI and subformula thereof, Y⁴ is C. In some embodiments of Formula CI and subformula thereof, R⁴ is hydrogen. In some embodiments of Formula CI and subformula thereof, Y⁵ is CH. In some embodiments of Formula CI and subformula thereof, Y⁶ is CH. In some embodiments of Formula CI and subformula thereof, Y¹ is C. In some embodiments of Formula CI and subformula thereof, Y² is C. In some embodiments of Formula CI and subformula thereof, Y³ is N. In some embodiments of Formula CI and subformula thereof, R³ is absent. In some embodiments of Formula CI and subformula thereof, Y⁷ is C.

In some embodiments, the RAS(ON) inhibitor has the structure of Formula CIc, or a pharmaceutically acceptable salt thereof:

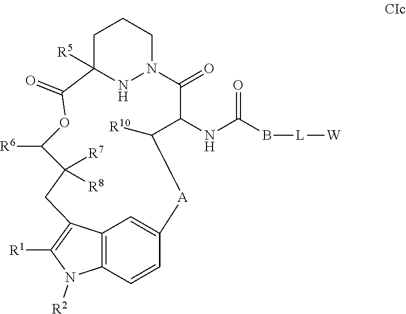

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
R¹⁰ is hydrogen, hydroxy, C₁-C₃ alkoxy, or C₁-C₃ alkyl.

In some embodiments of Formula CI and subformula thereof, R⁶ is hydrogen.

In some embodiments of Formula CI and subformula thereof, R² is hydrogen, cyano, optionally substituted C₁-C₆ alkyl, optionally substituted 3 to 6-membered cycloalkyl, or optionally substituted 3 to 6-membered heterocycloalkyl. In some embodiments, R² is optionally substituted C₁-C₆ alkyl, such as ethyl.

In some embodiments of Formula CI and subformula thereof, R⁷ is optionally substituted C₁-C₃ alkyl. In some embodiments, R⁷ is C₁-C₃ alkyl.

In some embodiments of Formula CI and subformula thereof, R⁸ is optionally substituted C₁-C₃ alkyl. In some embodiments, R⁸ is C₁-C₃ alkyl.

In some embodiments, the RAS(ON) inhibitor has the structure of Formula CId, or a pharmaceutically acceptable salt thereof:

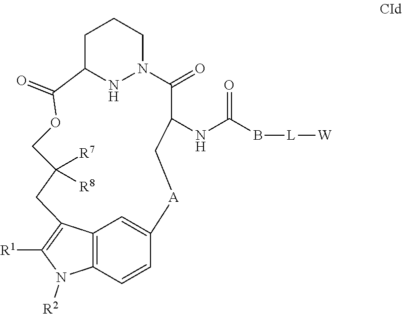

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula CI and subformula thereof, R¹ is 5 to 10-membered heteroaryl. In some embodiments, R¹ is optionally substituted 6-membered aryl or optionally substituted 6-membered heteroaryl.

In some embodiments, the RAS(ON) inhibitor has the structure of Formula Cle, or a pharmaceutically acceptable salt thereof:

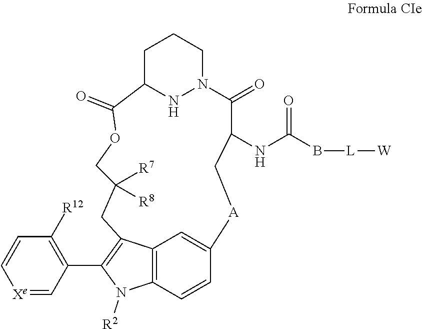

wherein A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl
X^{e} is N or CH; and
R¹² is optionally substituted C₁-C₆ alkyl or optionally substituted C₁-C₆ heteroalkyl.

In some embodiments of Formula CI and subformula thereof, X^{e} is N. In some embodiments, X^{e} is CH.

In some embodiments of Formula CI and subformula thereof, R¹² is optionally substituted C₁-C₆ heteroalkyl. In some embodiments, R¹² is,

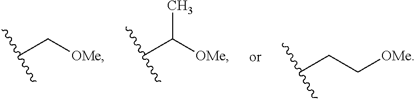

In some embodiments, R¹² is

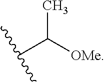 [SMILES: *C(C)OC]

In some embodiments, the RAS(ON) inhibitor has the structure of Formula CIf, or a pharmaceutically acceptable salt thereof:

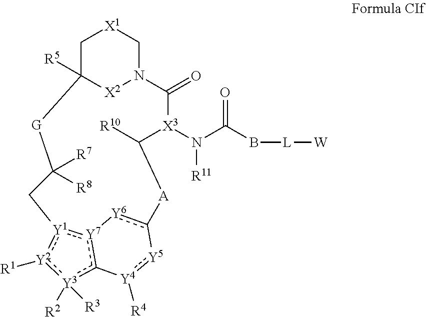

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, the RAS(ON) inhibitor has the structure of Formula CVI, or a pharmaceutically acceptable salt thereof:

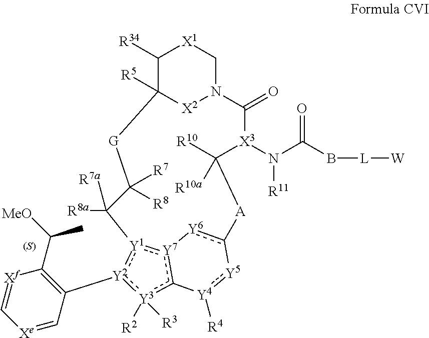

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene (e.g., phenyl or phenol), or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal: X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; R¹¹ is hydrogen or C₁-C₃ alkyl; R³⁴ is hydrogen or C₁-C₃ alkyl; and X^{e} and X^{f} are, independently, N or CH.

In some embodiments, the RAS(ON) inhibitor has the structure of Formula CVIa, or a pharmaceutically acceptable salt thereof:

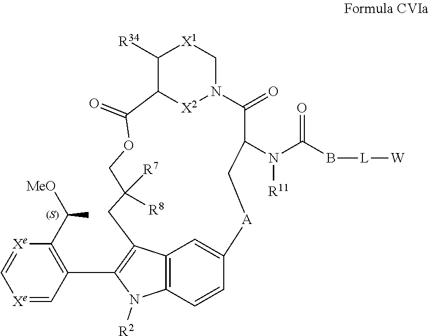

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{e} and X^{f} are, independently, N or CH;
R¹¹ is hydrogen or C₁-C₃ alkyl; and
R²¹ is hydrogen or C₁-C₃ alkyl.

In some embodiments of Formula CI and subformula thereof, X^{e} is N and X^{f} is CH. In some embodiments, X^{e} is CH and X^{f} is N.

In some embodiments, the RAS(ON) inhibitor has the structure of Formula CVIb, or a pharmaceutically acceptable salt thereof:

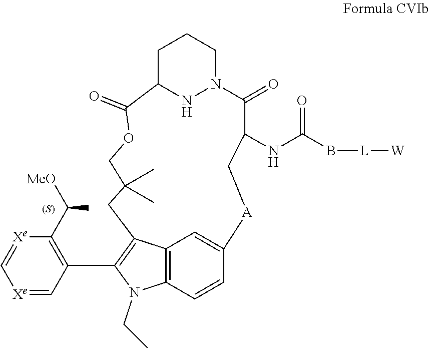

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
X^{e} and X^{f} are, independently, N or CH.

In some embodiments of Formula CI and subformula thereof, X^{e} is N and X^{f} is CH. In some embodiments, X^{e} is CH and X^{f} is N.

In some embodiments, the RAS(ON) inhibitor has the structure of Formula CVII, or a pharmaceutically acceptable salt thereof:

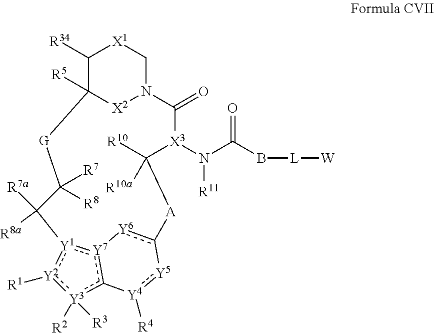

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is

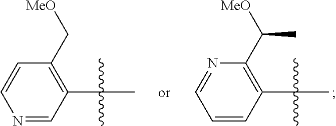 [SMILES: *c1cnccc1COC ; *c1cccnc1[C@H](C)OC]

R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl; R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R³⁴ is hydrogen or C₁-C₃ alkyl (e.g., methyl).

In some embodiments of Formula CI and subformula thereof, A is optionally substituted 6-membered arylene. In some embodiments, A has the structure:

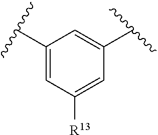

wherein R¹³ is hydrogen, hydroxy, amino, optionally substituted C₁-C₆ alkyl, or optionally substituted C₁-C₆ heteroalkyl. In some embodiments, R¹³ is hydrogen. In some embodiments, R¹³ is hydroxy.

In some embodiments of Formula CI and subformula thereof, B is —CHR⁹—. In some embodiments, R⁹ is optionally substituted C₁-C₆ alkyl or optionally substituted 3 to 6-membered cycloalkyl. In some embodiments, R⁹ is:

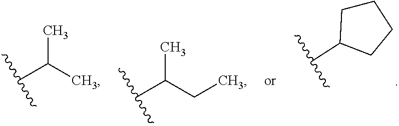

In some embodiments, R⁹ is:

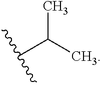 [SMILES: *C(C)C]

In some embodiments, R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments of Formula CI and subformula thereof, B is optionally substituted 6-membered arylene. In some embodiments, B is 6-membered arylene. In some embodiments, B is:

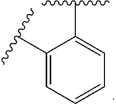 [SMILES: *c1ccccc1*]

In some embodiments of Formula CI and subformula thereof, R⁷ is methyl.

In some embodiments of Formula CI and subformula thereof, R⁸ is methyl.

In some embodiments of Formula CI and subformula thereof, R³⁴ is hydrogen.

In some embodiments of Formula CI and subformula thereof, the linker is the structure of Formula CII:

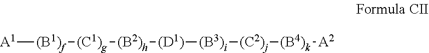

where A¹ is a bond between the linker and B; A² is a bond between Wand the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C₁ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments of Formula CI and subformula thereof, the linker is acyclic. In some embodiments of Formula CI and subformula thereof, the linker has the structure of Formula CIIa:

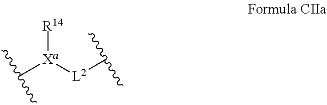

wherein X^{a} is absent or N;
R¹⁴ is absent, hydrogen or optionally substituted C₁-C₆ alkyl; and
L² is absent, —SO₂—, optionally substituted C₁-C₄ alkylene or optionally substituted C₁-C₄ heteroalkylene, wherein at least one of X^{a}, R¹⁴, or L² is present. In some embodiments, the linker has the structure:

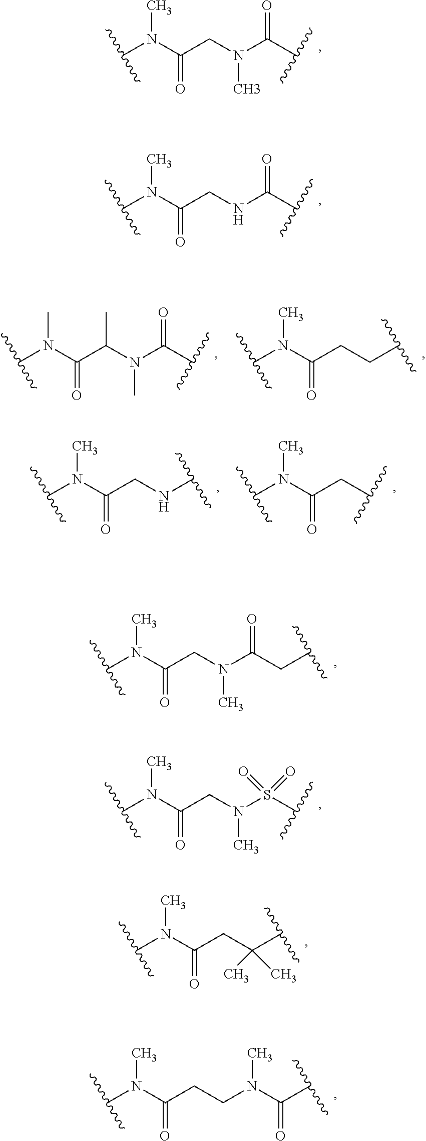

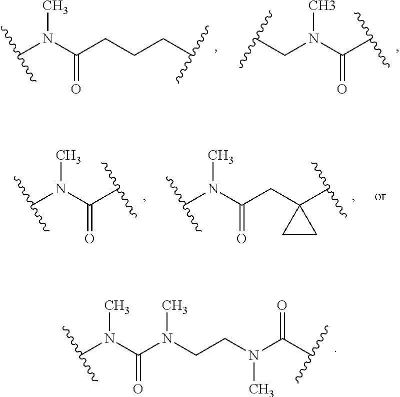

In some embodiments of Formula CI and subformula thereof, the linker is or a comprises a cyclic group. In some embodiments, the linker has the structure of Formula CIIb:

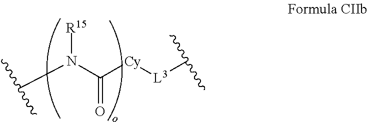

wherein o is 0 or 1;
R¹⁵ is hydrogen or optionally substituted C₁-C₆ alkyl;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —SO₂—, optionally substituted C₁-C₄ alkylene or optionally substituted C₁-C₄ heteroalkylene. In some embodiments, the linker has the structure:

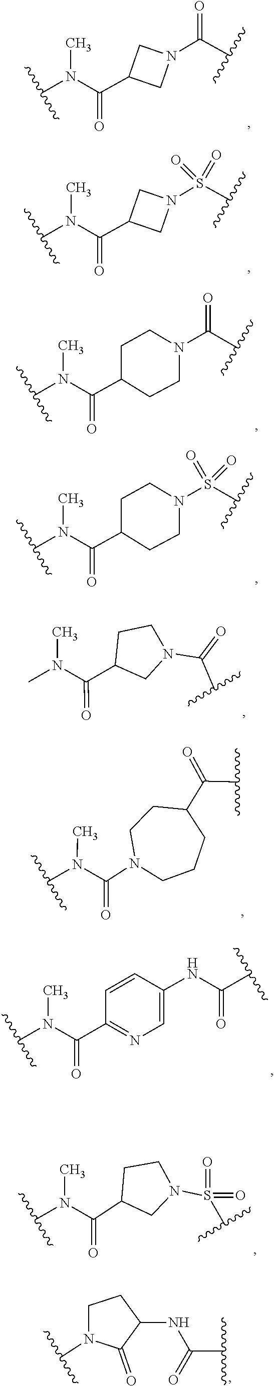

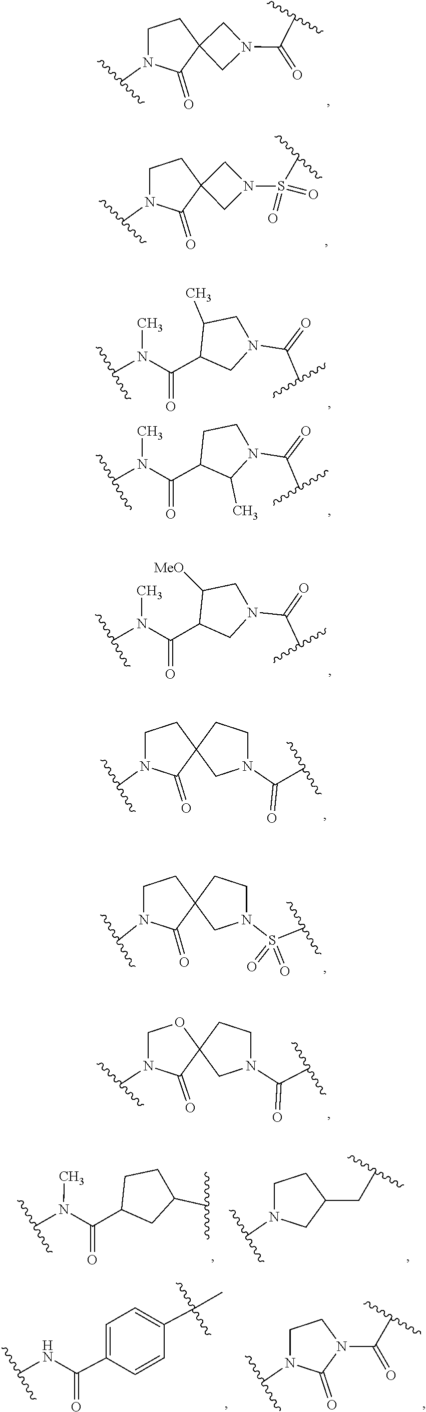

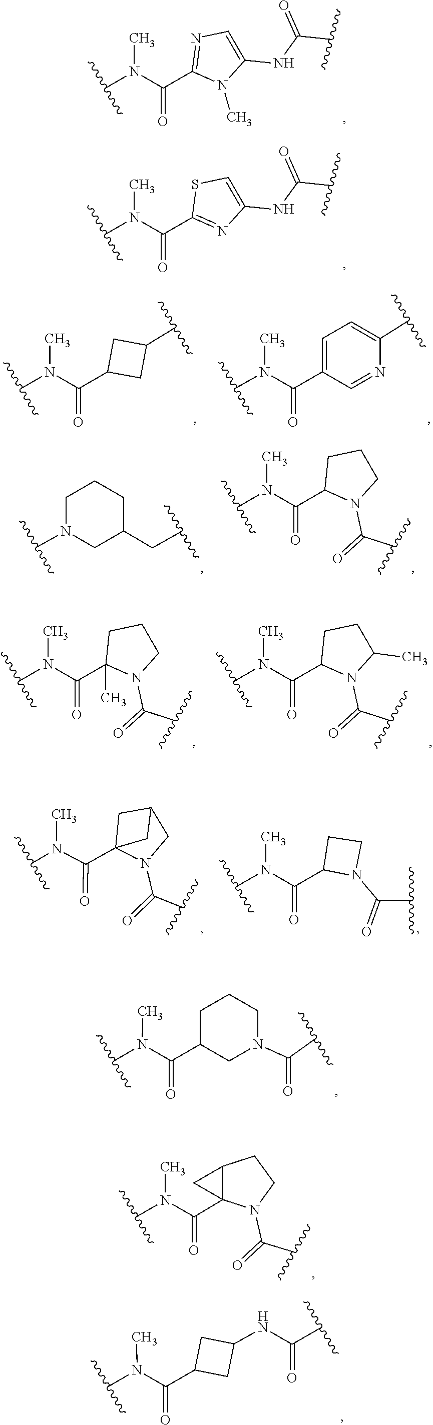

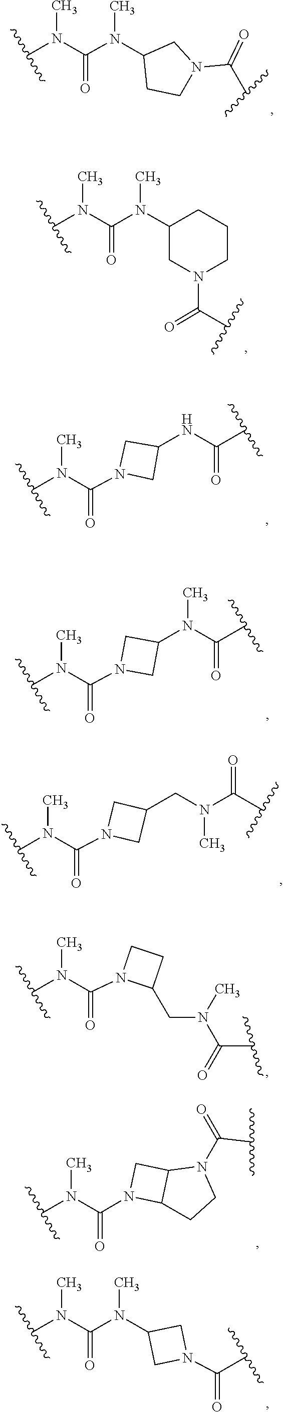

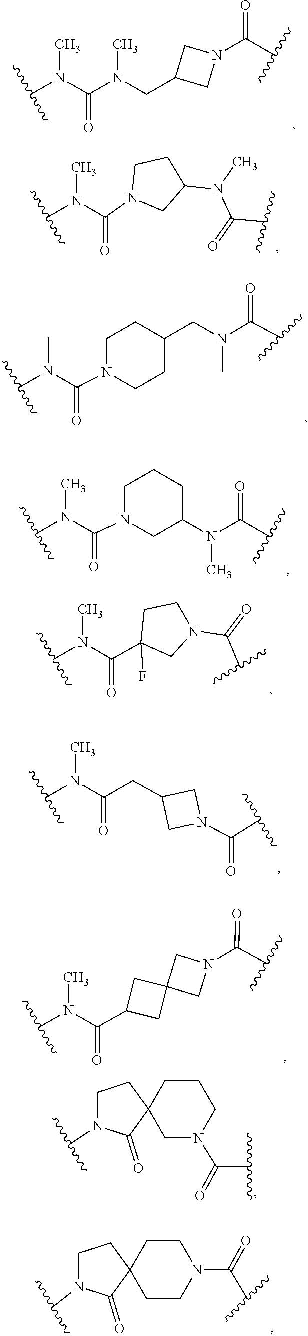

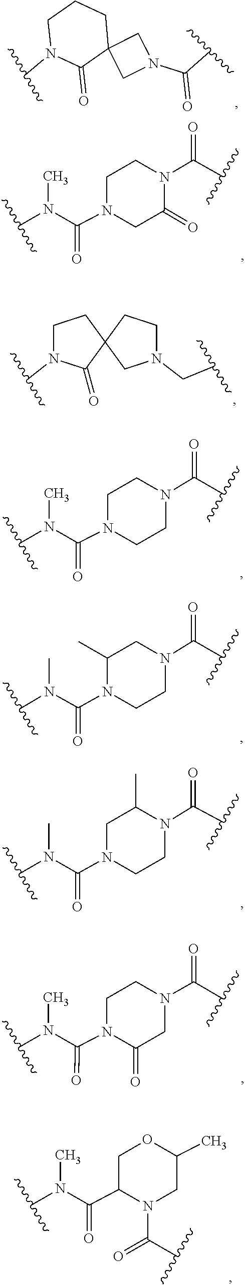

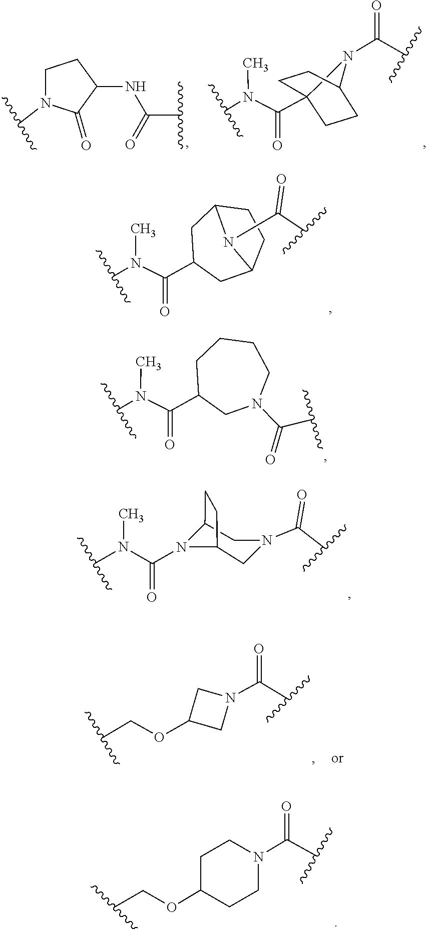

In some embodiments, a linker of Formula CII is selected from the group consisting of

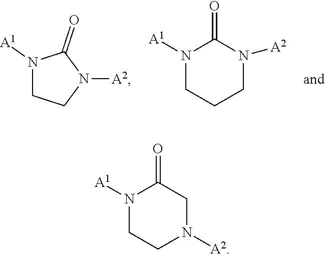

In some embodiments of Formula CI and subformula thereof, W comprises a carbodiimide. In some embodiments, W has the structure of Formula CIIIa:

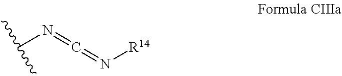

wherein R¹⁴ is optionally substituted C₁-C₆ alkyl, optionally substituted C₁-C₆ heteroalkyl, optionally substituted 3 to 8-membered cycloalkyl, optionally substituted 6 to 10-membered aryl, optionally substituted 3 to 14-membered heterocycloalkyl, or optionally substituted 5 to 10-membered heteroaryl. In some embodiments, W has the structure:

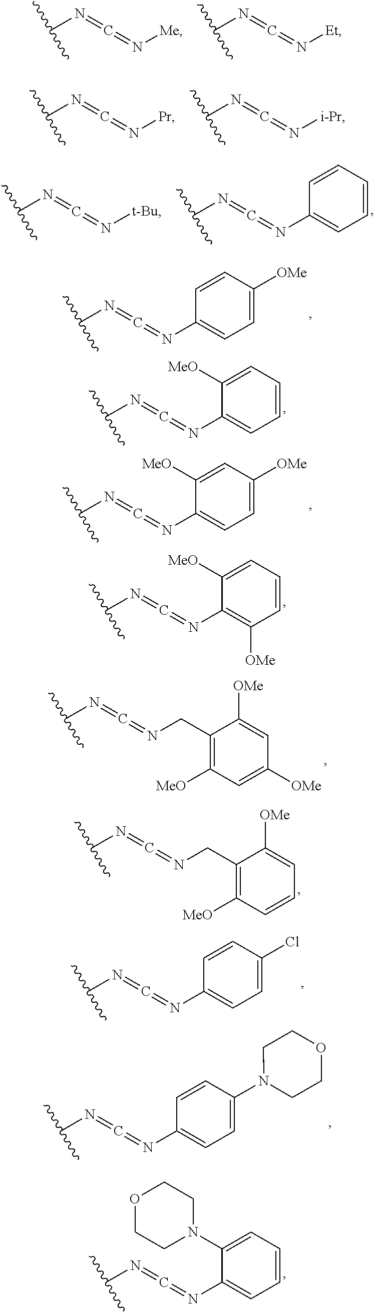

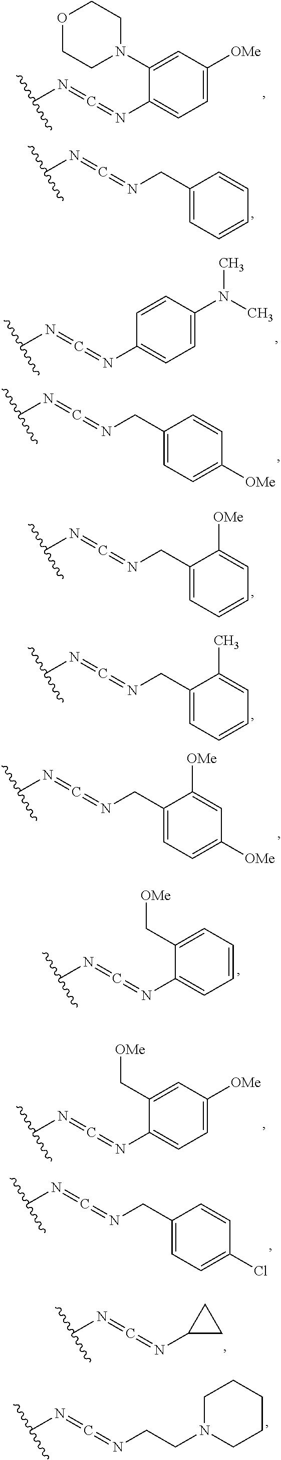

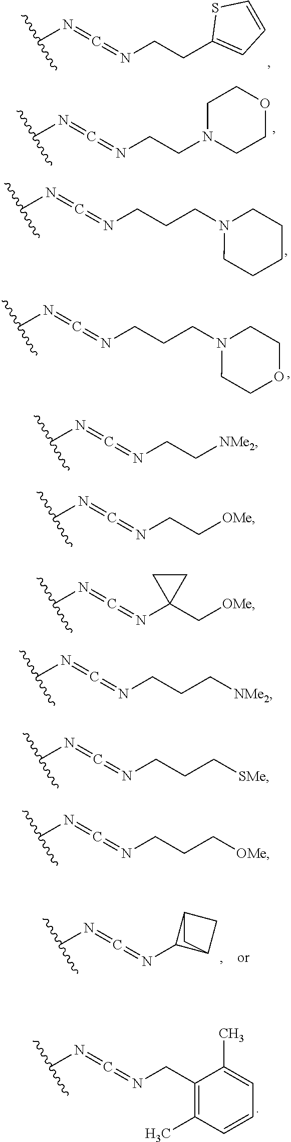

In some embodiments of Formula CI and subformula thereof, W comprises an oxazoline or thiazoline. In some embodiments, W has the structure of Formula CIIb:

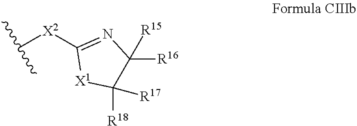

wherein X¹ is O or S;
X² is absent or NR¹⁹
R¹⁵, R¹⁶, R¹⁷, and R¹⁸ are, independently, hydrogen or optionally substituted C₁-C₆ alkyl; and
R¹⁹ is hydrogen, C(O)(optionally substituted C₁-C₆ alkyl), optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is

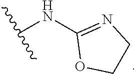 [SMILES: *NC1=NCCO1]

In some embodiments of Formula CI and subformula thereof, W comprises a chloroethyl urea, a chloroethyl thiourea, a chloroethyl carbamate, or a chloroethyl thiocarbamate. In some embodiments, W has the structure of Formula CIIIc:

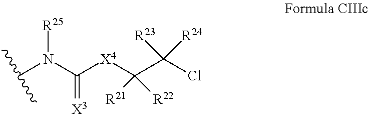

wherein X³ is O or S;
X⁴ is O, S, NR²⁶;
R²¹, R²², R²³, R²⁴ and R²⁶ are, independently, hydrogen or optionally substituted C₁-C₆ alkyl; and
R²⁵ is hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is

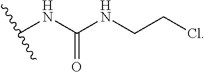 [SMILES: *NC(=O)NCCCl]

In some embodiments of Formula CI and subformula thereof, W comprises an aziridine. In some embodiments, W has the structure of Formula CIIId1, Formula CIIId2, Formula CIIId3, or Formula CIIId4:

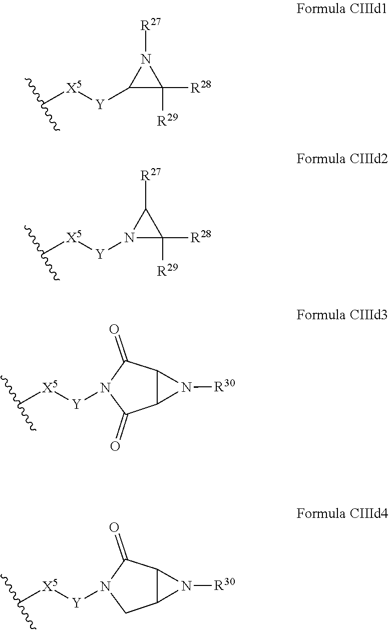

wherein X⁵ is absent or NR³⁰;
Y is absent or C(O), C(S), S(O), SO₂, or optionally substituted C₁-C₃ alkylene;
R²⁷ is hydrogen, —C(O)R³², —C(O)OR³², —SOR³³, —SO₂R³³, optionally substituted C₁-C₆ alkyl, optionally substituted 6 to 10-membered aryl, optionally substituted 3 to 14-membered heterocycloalkyl, or optionally substituted 5 to 10-membered heteroaryl;
R²⁸ and R²⁹ are, independently, hydrogen, CN, C(O)R³¹, C₀₂R³¹, C(O)R³¹R³¹ optionally substituted C₁-C₆ alkyl, optionally substituted 3 to 10-membered cycloalkyl, optionally substituted 6 to 10-membered aryl, optionally substituted 3 to 14-membered heterocycloalkyl, or optionally substituted 5 to 10-membered heteroaryl;
each R³¹ is, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl;
R³⁰ is hydrogen or optionally substituted C₁-C₆ alkyl; and
R³² and R³³ are, independently, hydrogen, optionally substituted C₁-C₆ alkyl, optionally substituted 6 to 10-membered aryl, optionally substituted 3 to 14-membered heterocycloalkyl, or optionally substituted 5 to 10-membered heteroaryl. In some embodiments, W is:

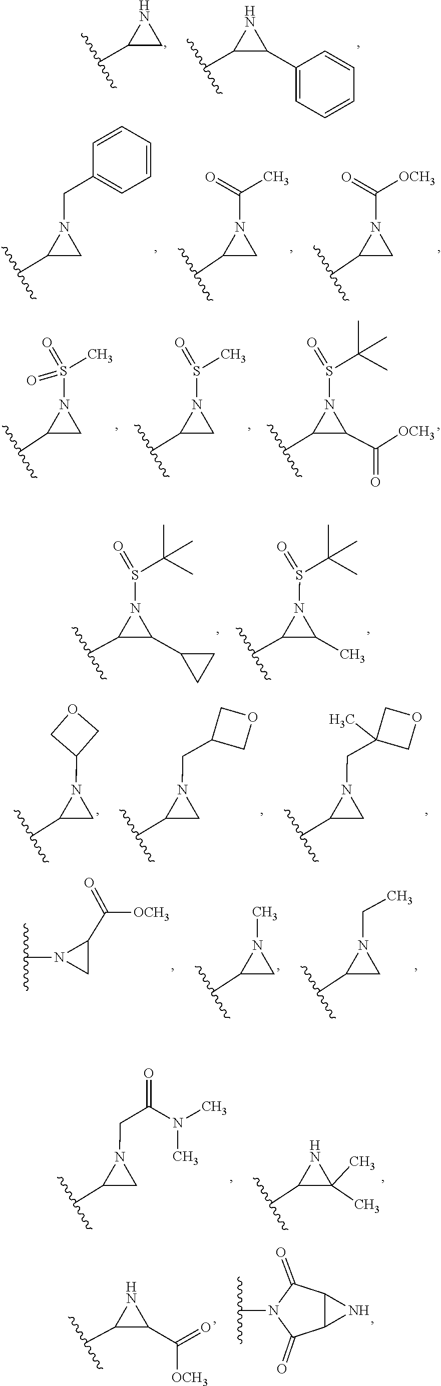

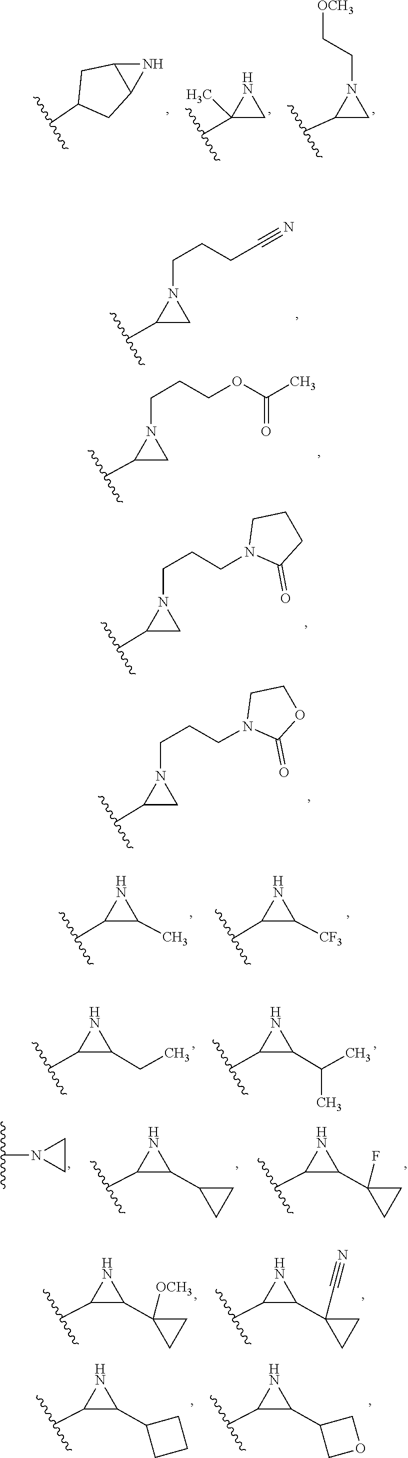

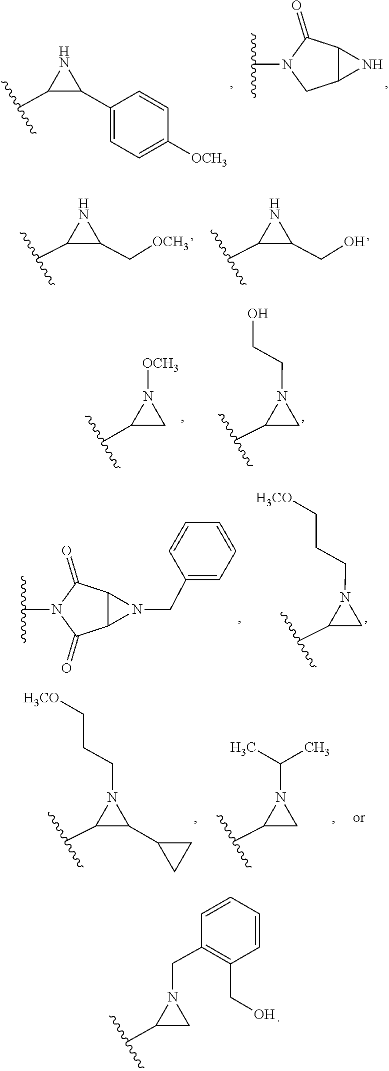

In some embodiments of Formula CI and subformula thereof, W comprises an epoxide. In some embodiments, W is

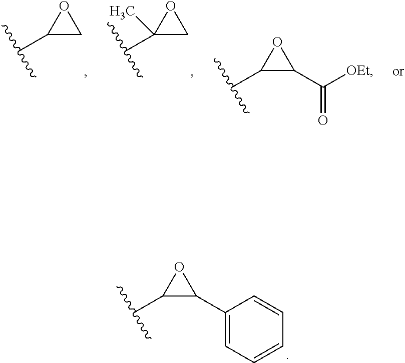

In some embodiments of Formula CI and subformula thereof, W is a cross-linking group bound to an organic moiety that is a Ras binding moiety, i.e., RBM-W, wherein upon contact of an RBM-W compound with a Ras protein, the RBM-W binds to the Ras protein to form a conjugate. For example, the W moiety of an RBM-W compound may bind, e.g., cross-link, with an amino acid of the Ras protein to form the conjugate. In some embodiments, the Ras binding moiety is a K-Ras binding moiety. In some embodiments, the K-Ras binding moiety binds to a residue of a K-Ras Switch-II binding pocket of the K-Ras protein. In some embodiments, the Ras binding moiety is an H-Ras binding moiety that binds to a residue of an H-Ras Switch-II binding pocket of an H-Ras protein. In some embodiments, the Ras binding moiety is an N-Ras binding moiety that binds to a residue of an N-Ras Switch-II binding pocket of an N-Ras protein. The W of an RBM-W compound may comprise any W described herein. The Ras binding moiety typically has a molecular weight of under 1200 Da. See, e.g., see, e.g., Johnson et al., 292:12981-12993 (2017) for a description of Ras protein domains, incorporated herein by reference. In some embodiments, the RAS(ON) inhibitor is selected from Table C1, or a pharmaceutically acceptable salt or stereoisomer thereof. In some embodiments, the RAS(ON) inhibitor is selected from Table C1, or a pharmaceutically acceptable salt or atropisomer thereof.

TABLE C1
Certain Compounds of the Present Invention

| Ex# | Structure |
|---|---|
| C1 | 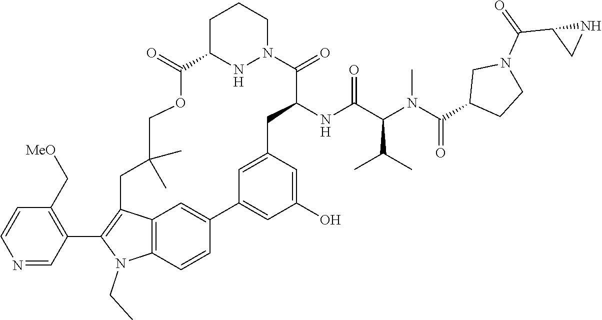 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@H]3CN3)C1)C(=O)N1CCC[C@H](N1)C(=O)OCC(C)(C)C2] |
| C2 | 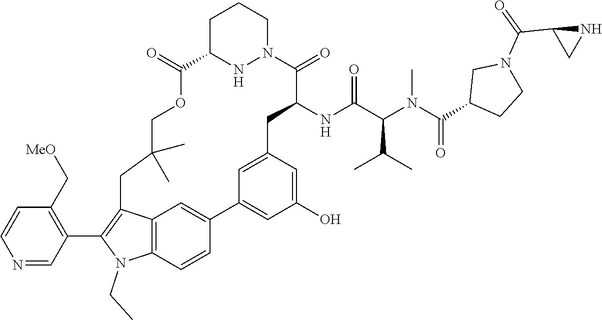 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@@H]3CN3)C1)C(=O)N1CCC[C@H](N1)C(=O)OCC(C)(C)C2] |
| C3 | 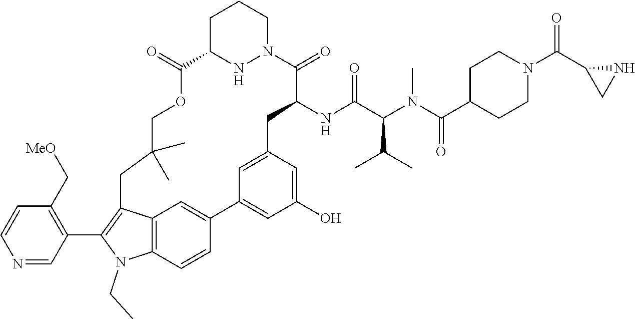 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)[C@H]3CN3)CC1)C(=O)N1CCC[C@H](N1)C(=O)OCC(C)(C)C2] |
| C4 | 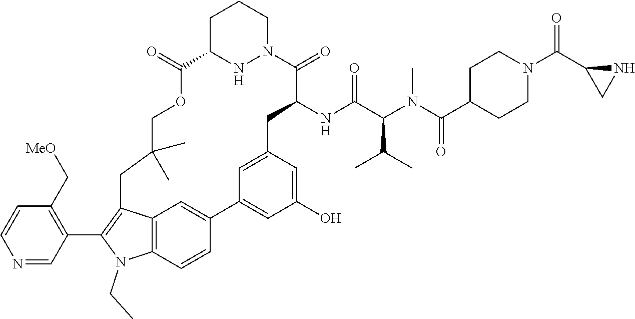 [SMILES: CCn1c(-c2cnccc2COC)c2c3cc(ccc31)-c1cc(O)cc(c1)C[C@H](NC(=O)[C@H](C(C)C)N(C)C(=O)C1CCN(C(=O)[C@@H]3CN3)CC1)C(=O)N1CCC[C@H](N1)C(=O)OCC(C)(C)C2] |
| C5 | 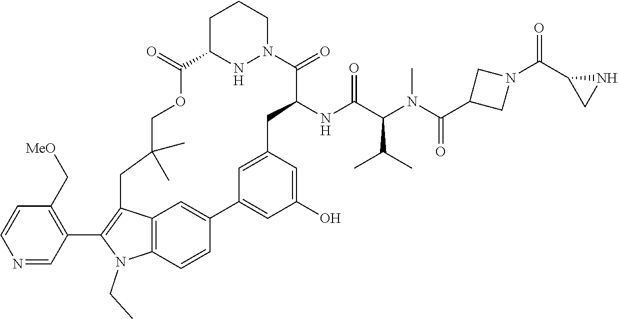 [SMILES: CCn1c(-c2cnccc2COC)c2c3cc(ccc31)-c1cc(O)cc(c1)C[C@H](NC(=O)[C@H](C(C)C)N(C)C(=O)C1CN(C(=O)[C@H]3CN3)C1)C(=O)N1CCC[C@H](N1)C(=O)OCC(C)(C)C2] |
| C6 | 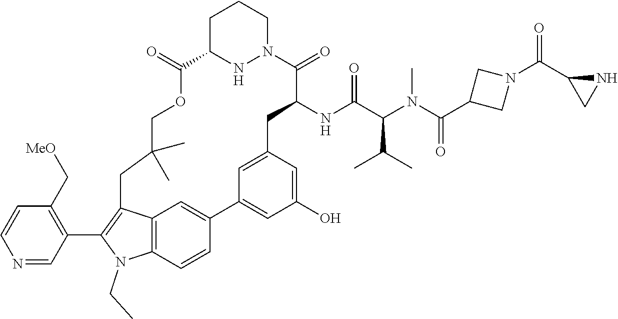 [SMILES: CCn1c(-c2cnccc2COC)c2c3cc(ccc31)-c1cc(O)cc(c1)C[C@H](NC(=O)[C@H](C(C)C)N(C)C(=O)C1CN(C(=O)[C@@H]3CN3)C1)C(=O)N1CCC[C@H](N1)C(=O)OCC(C)(C)C2] |
| C7 | 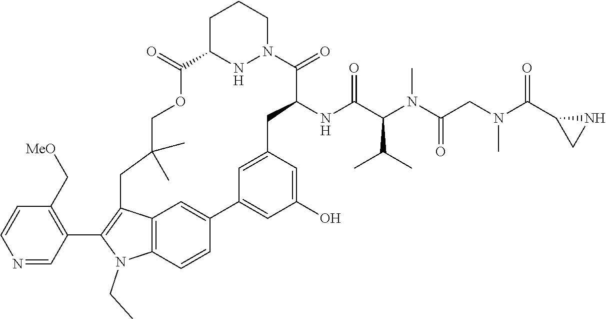 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1)C(=O)N1CCC[C@H](N1)C(=O)OCC(C)(C)C2] |
| C8 | 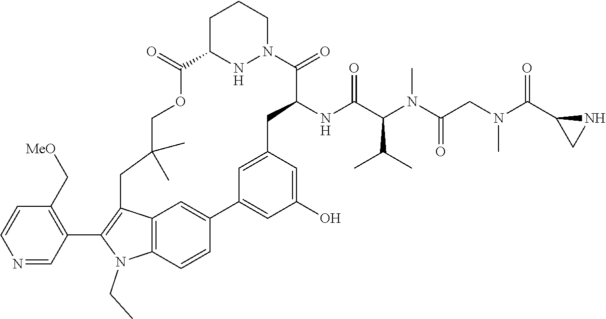 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1)C(=O)N1CCC[C@H](N1)C(=O)OCC(C)(C)C2] |
| C9 | 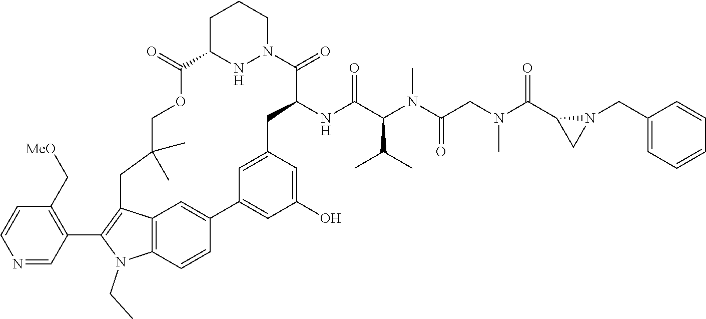 |
| C10 | 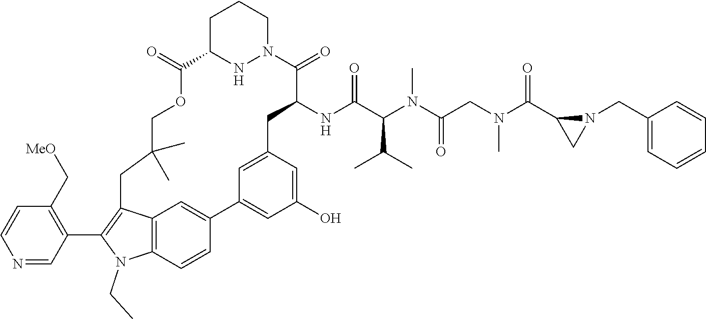 |
| C11 | 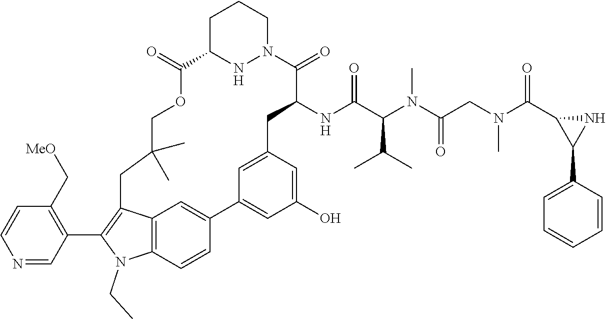 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1c1ccccc1)C(=O)N1CCC[C@H](N1)C(=O)OCC(C)(C)C2] |
| C12 | 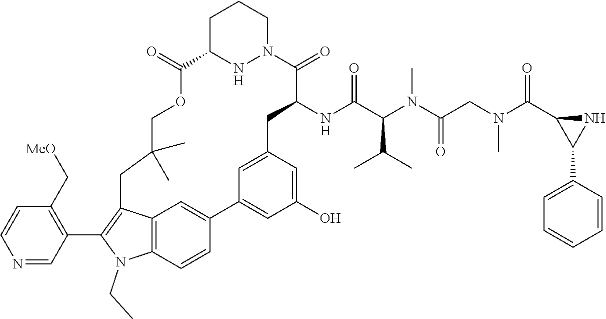 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1c1ccccc1)C(=O)N1CCC[C@H](N1)C(=O)OCC(C)(C)C2] |
| C13 | 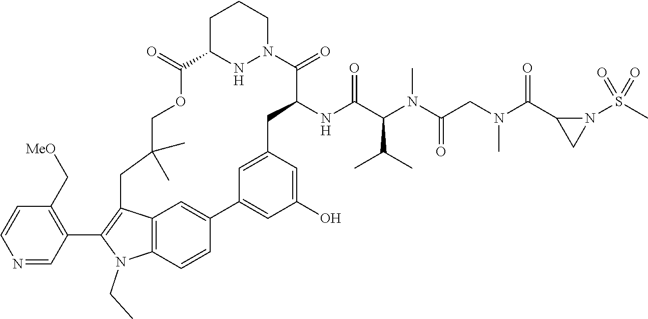 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1S(C)(=O)=O)C(=O)N1CCC[C@H](N1)C(=O)OCC(C)(C)C2] |
| C14 | 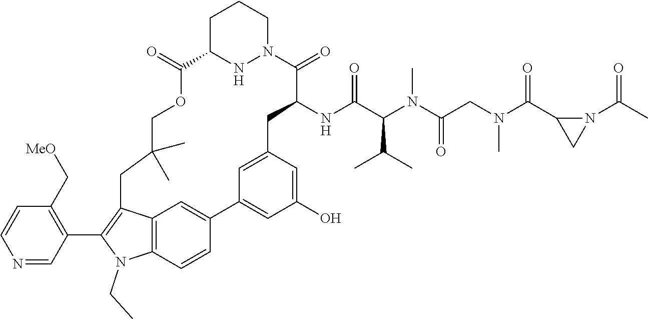 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1C(C)=O)C(=O)N1CCC[C@H](N1)C(=O)OCC(C)(C)C2] |
| C15 A and B | 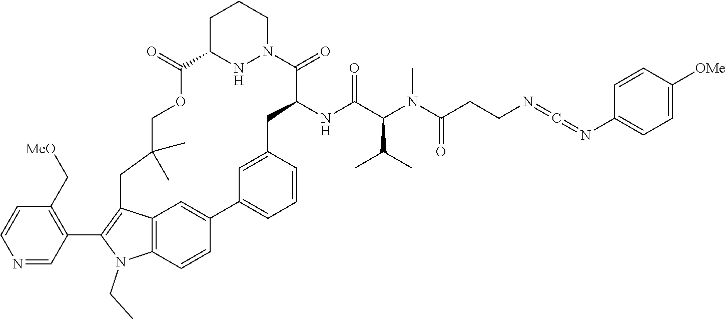 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1)C(=O)N1CCC[C@H](N1)C(=O)OCC(C)(C)C2] |
| C16 A and B | 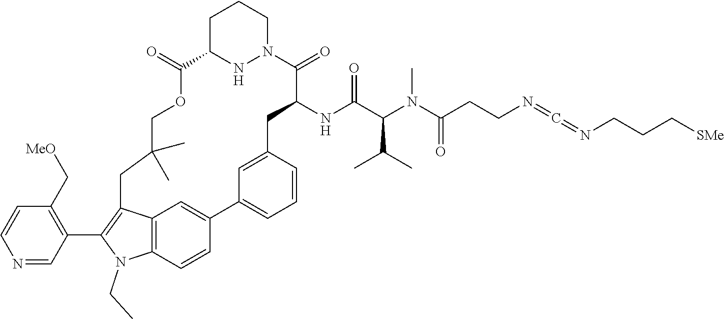 [SMILES: CCn1c(-c2cnccc2COC)c2c3cc(ccc31)-c1cccc(c1)C[C@H](NC(=O)[C@H](C(C)C)N(C)C(=O)CCN=C=NCCCSC)C(=O)N1CCC[C@H](N1)C(=O)OCC(C)(C)C2] |
| C17 A and B | 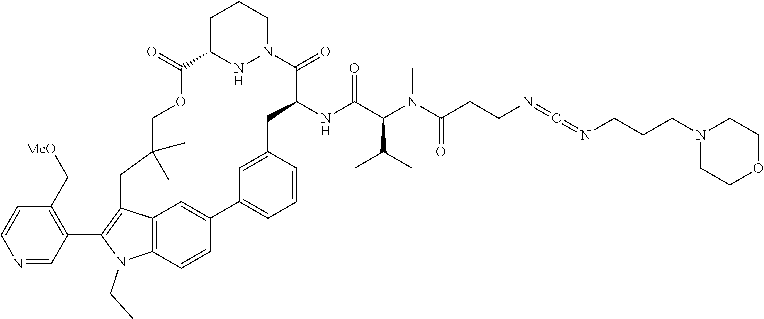 [SMILES: CCn1c(-c2cnccc2COC)c2c3cc(ccc31)-c1cccc(c1)C[C@H](NC(=O)[C@H](C(C)C)N(C)C(=O)CCN=C=NCCCN1CCOCC1)C(=O)N1CCC[C@H](N1)C(=O)OCC(C)(C)C2] |
| C18 A and B | 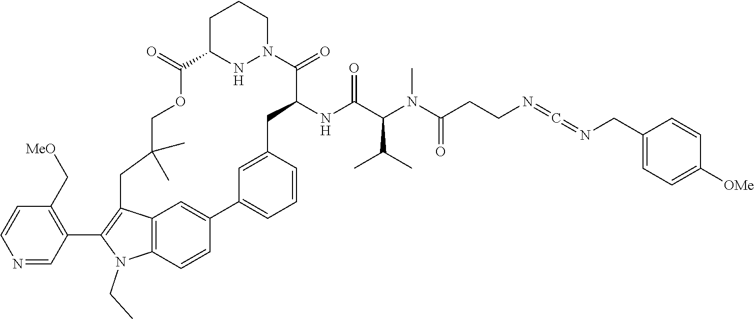 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccc(OC)cc1)C(=O)N1CCC[C@H](N1)C(=O)OCC(C)(C)C2] |
| C19 A and B | 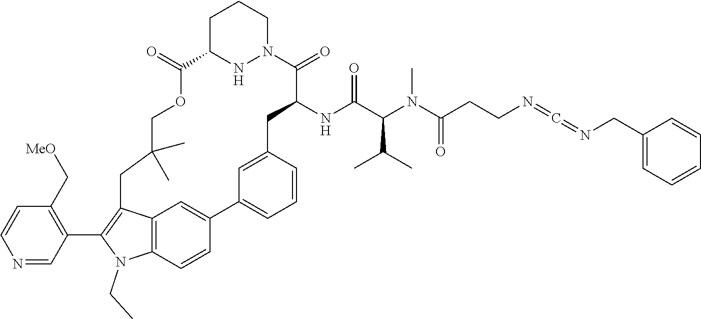 [SMILES: CCn1c(-c2cnccc2COC)c2c3cc(ccc31)-c1cccc(c1)C[C@H](NC(=O)[C@H](C(C)C)N(C)C(=O)CCN=C=NCc1ccccc1)C(=O)N1CCC[C@H](N1)C(=O)OCC(C)(C)C2] |
| C20 A and B | 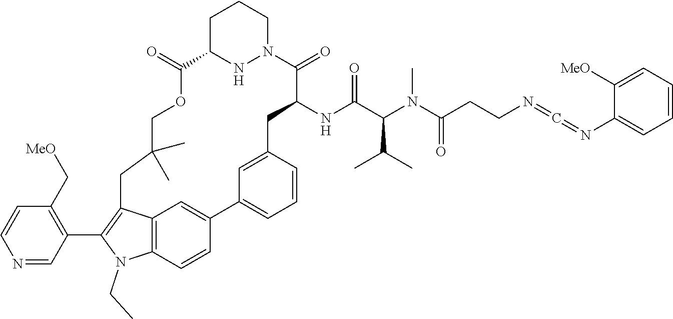 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1OC)C(=O)N1CCC[C@H](N1)C(=O)OCC(C)(C)C2] |
| C21 A and B | 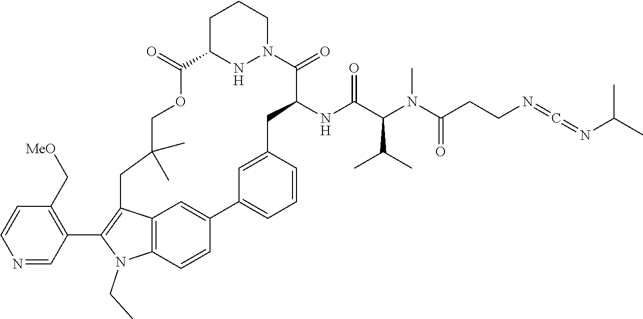 [SMILES: CCn1c(-c2cnccc2COC)c2c3cc(ccc31)-c1cccc(c1)C[C@H](NC(=O)[C@H](C(C)C)N(C)C(=O)CCN=C=NC(C)C)C(=O)N1CCC[C@H](N1)C(=O)OCC(C)(C)C2] |
| C22 A and B | 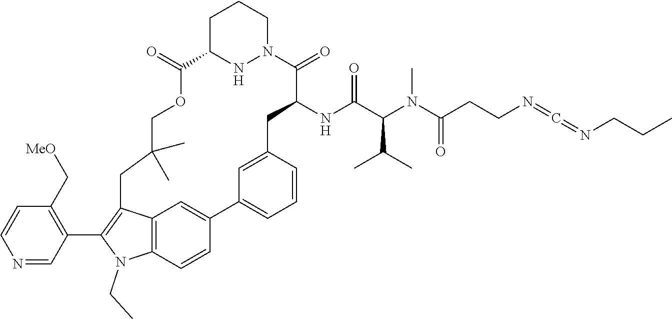 [SMILES: CCCN=C=NCCC(=O)N(C)[C@H](C(=O)N[C@H]1Cc2cccc(c2)-c2ccc3c(c2)c(c(-c2cnccc2COC)n3CC)CC(C)(C)COC(=O)[C@@H]2CCCN(N2)C1=O)C(C)C] |
| C23 A and B | 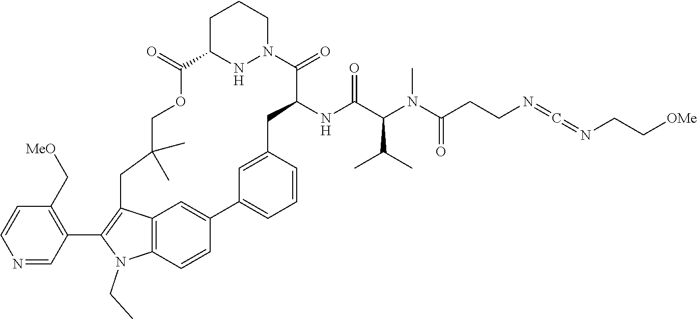 [SMILES: CCn1c(-c2cnccc2COC)c2c3cc(ccc31)-c1cccc(c1)C[C@H](NC(=O)[C@H](C(C)C)N(C)C(=O)CCN=C=NCCOC)C(=O)N1CCC[C@H](N1)C(=O)OCC(C)(C)C2] |
| C24 A and B | 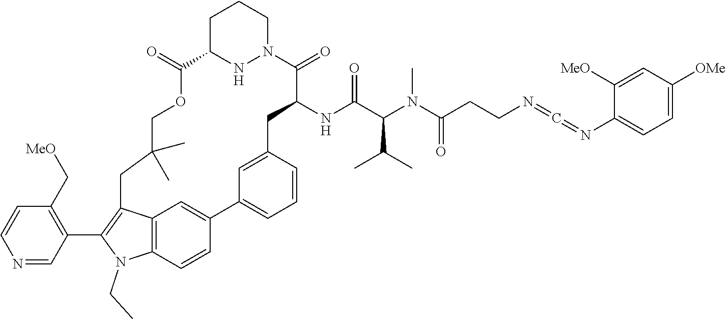 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1OC)C(=O)N1CCC[C@H](N1)C(=O)OCC(C)(C)C2] |
| C25 | 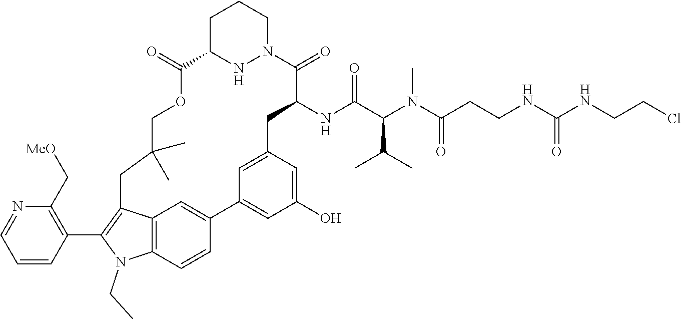 [SMILES: CCn1c(-c2cccnc2COC)c2c3cc(ccc31)-c1cc(O)cc(c1)C[C@H](NC(=O)[C@H](C(C)C)N(C)C(=O)CCNC(=O)NCCCl)C(=O)N1CCC[C@H](N1)C(=O)OCC(C)(C)C2] |
| C26 | 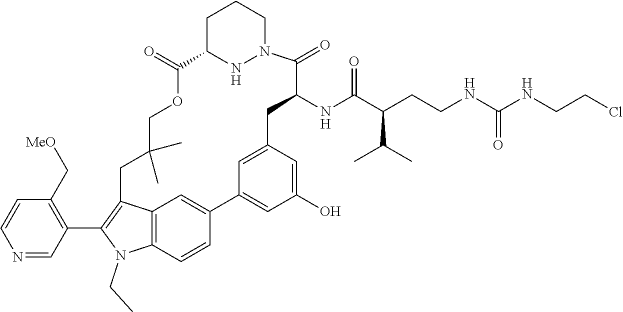 [SMILES: CCn1c(-c2cnccc2COC)c2c3cc(ccc31)-c1cc(O)cc(c1)C[C@H](NC(=O)[C@@H](CCNC(=O)NCCCl)C(C)C)C(=O)N1CCC[C@H](N1)C(=O)OCC(C)(C)C2] |
| C27 | 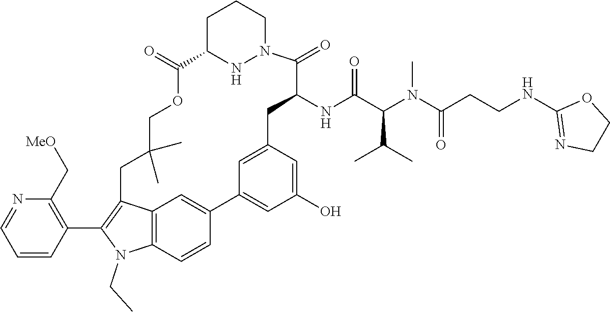 [SMILES: CCn1c(-c2cccnc2COC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2] |
| C28 | 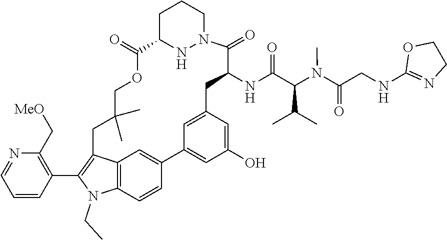 [SMILES: CCn1c(-c2cccnc2COC)c2c3cc(ccc31)-c1cc(O)cc(c1)C[C@H](NC(=O)[C@H](C(C)C)N(C)C(=O)CNC1=NCCO1)C(=O)N1CCC[C@H](N1)C(=O)OCC(C)(C)C2] |
| C29 | 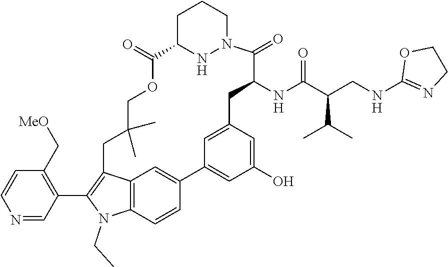 [SMILES: CCn1c(-c2cnccc2COC)c2c3cc(ccc31)-c1cc(O)cc(c1)C[C@H](NC(=O)[C@@H](CNC1=NCCO1)C(C)C)C(=O)N1CCC[C@H](N1)C(=O)OCC(C)(C)C2] |
| C30 | 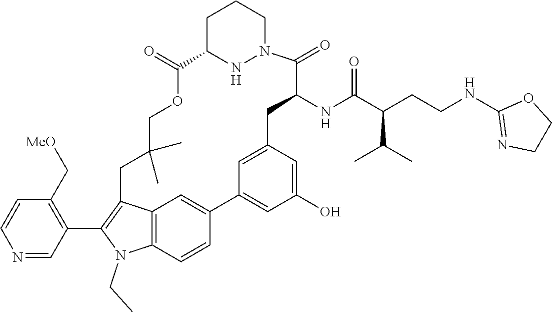 [SMILES: CCn1c(-c2cnccc2COC)c2c3cc(ccc31)-c1cc(O)cc(c1)C[C@H](NC(=O)[C@@H](CCNC1=NCCO1)C(C)C)C(=O)N1CCC[C@H](N1)C(=O)OCC(C)(C)C2] |
| C31 | 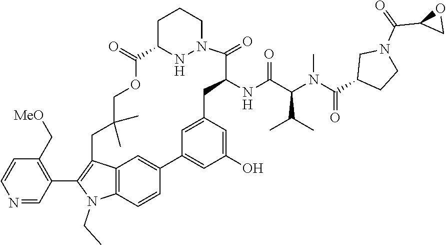 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)[C@@H]3CO3)C1)C(=O)N1CCC[C@H](N1)C(=O)OCC(C)(C)C2] |
| C32 A and B | 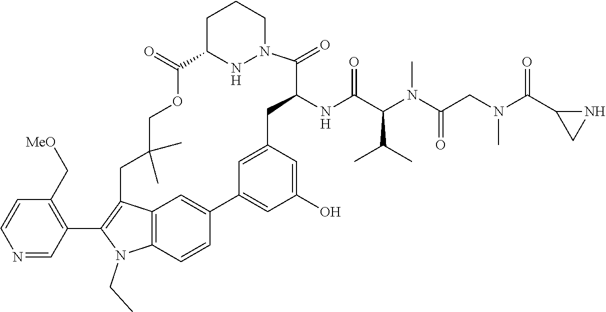 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1)C(=O)N1CCC[C@H](N1)C(=O)OCC(C)(C)C2] |
| C33 | 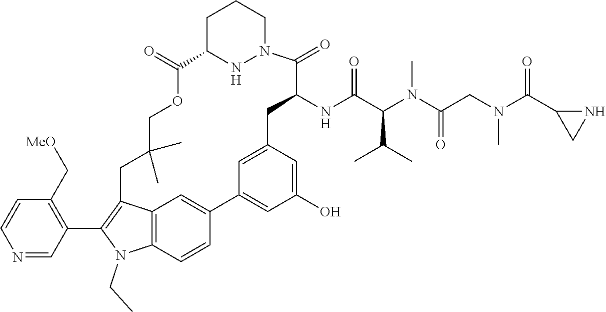 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)C1CN1)C(=O)N1CCC[C@H](N1)C(=O)OCC(C)(C)C2] |
| C34 | 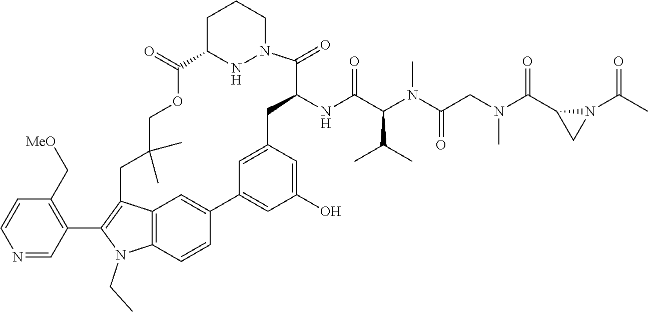 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1C(C)=O)C(=O)N1CCC[C@H](N1)C(=O)OCC(C)(C)C2] |
| C35 | 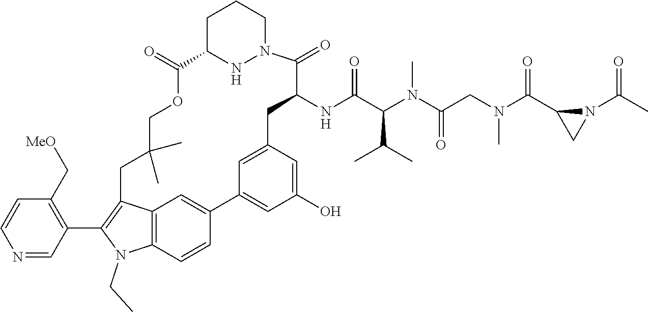 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1C(C)=O)C(=O)N1CCC[C@H](N1)C(=O)OCC(C)(C)C2] |
| C36 | 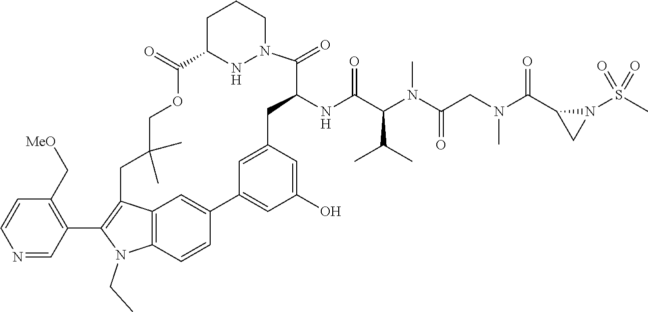 |
| C37 | 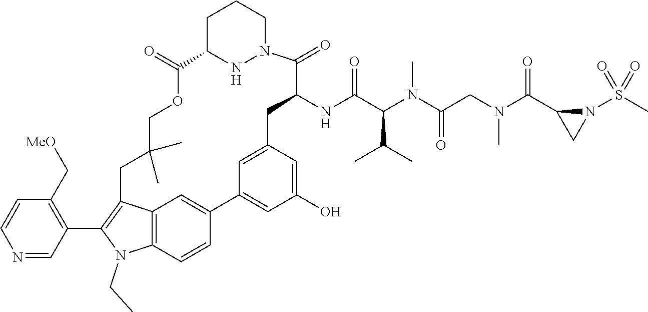 |
| C38 | 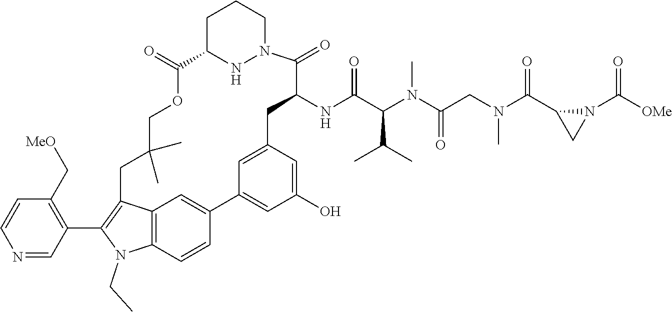 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1CN1C(=O)OC)C(=O)N1CCC[C@H](N1)C(=O)OCC(C)(C)C2] |
| C39 | 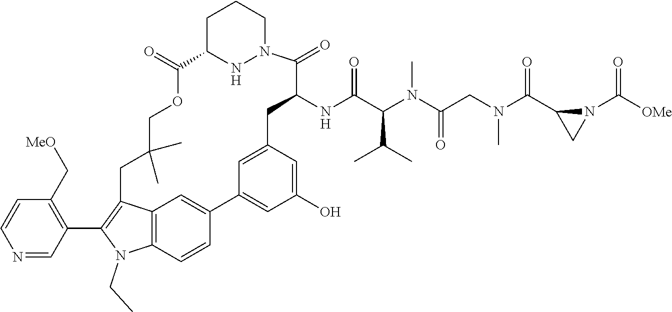 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1CN1C(=O)OC)C(=O)N1CCC[C@H](N1)C(=O)OCC(C)(C)C2] |
| C40 | 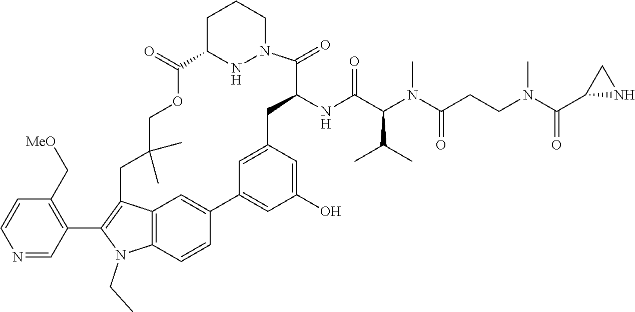 [SMILES: CCn1c(-c2cnccc2COC)c2c3cc(ccc31)-c1cc(O)cc(c1)C[C@H](NC(=O)[C@H](C(C)C)N(C)C(=O)CCN(C)C(=O)[C@@H]1CN1)C(=O)N1CCC[C@H](N1)C(=O)OCC(C)(C)C2] |
| C41 | 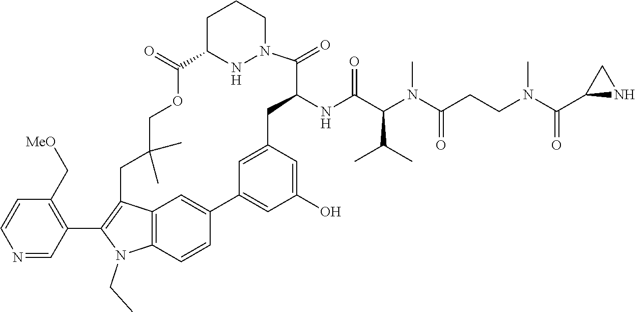 [SMILES: CCn1c(-c2cnccc2COC)c2c3cc(ccc31)-c1cc(O)cc(c1)C[C@H](NC(=O)[C@H](C(C)C)N(C)C(=O)CCN(C)C(=O)[C@H]1CN1)C(=O)N1CCC[C@H](N1)C(=O)OCC(C)(C)C2] |
| C42A | 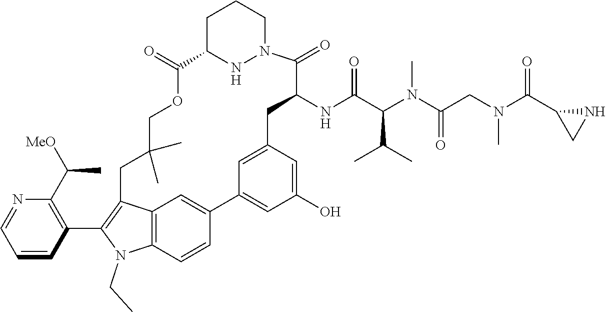 |
| C42B | 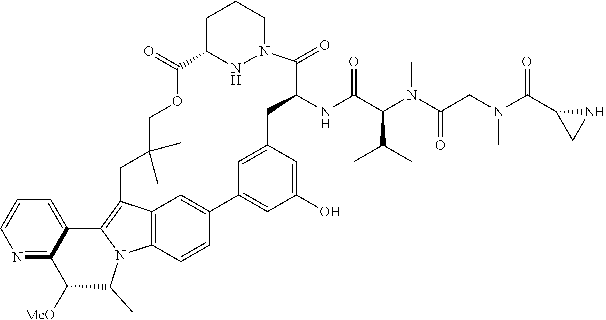 |
| C43A | 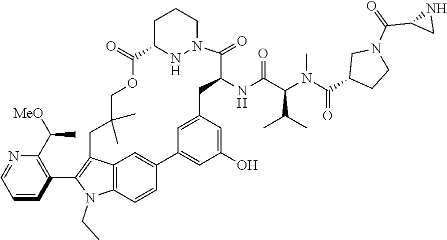 |
| C43B | 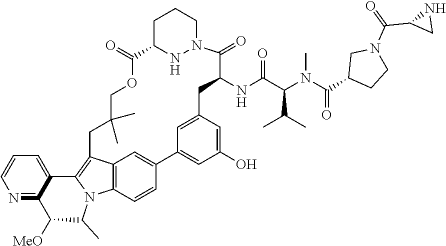 |
| C44 | 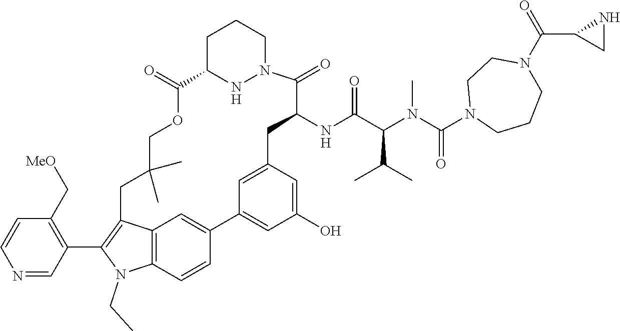 [SMILES: CCn1c(-c2cnccc2COC)c2c3cc(ccc31)-c1cc(O)cc(c1)C[C@H](NC(=O)[C@H](C(C)C)N(C)C(=O)N1CCCN(C(=O)[C@H]3CN3)CC1)C(=O)N1CCC[C@H](N1)C(=O)OCC(C)(C)C2] |
| C45 | 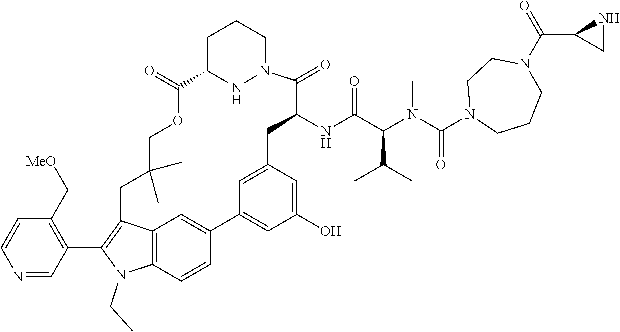 [SMILES: CCn1c(-c2cnccc2COC)c2c3cc(ccc31)-c1cc(O)cc(c1)C[C@H](NC(=O)[C@H](C(C)C)N(C)C(=O)N1CCCN(C(=O)[C@@H]3CN3)CC1)C(=O)N1CCC[C@H](N1)C(=O)OCC(C)(C)C2] |
| C46 | 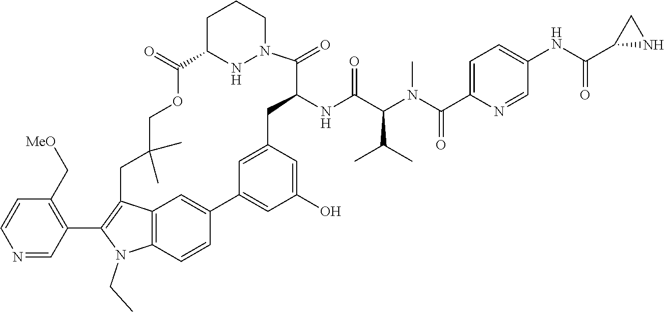 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3CN3)cn1)C(=O)N1CCC[C@H](N1)C(=O)OCC(C)(C)C2] |
| C47 A and B | 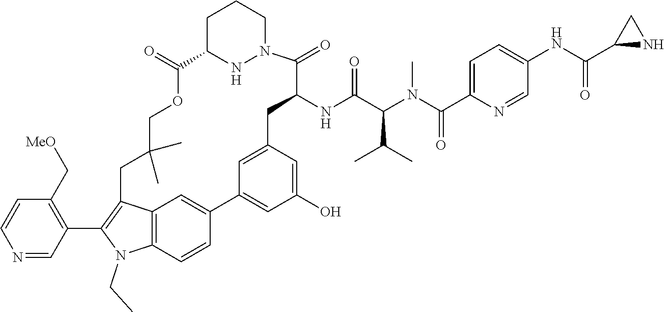 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3CN3)cn1)C(=O)N1CCC[C@H](N1)C(=O)OCC(C)(C)C2] |
| C48 | 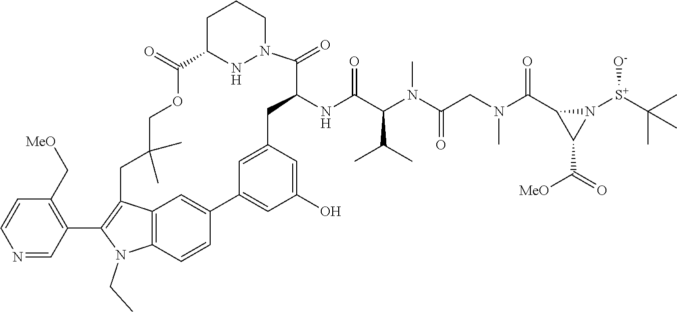 |
| C49 | 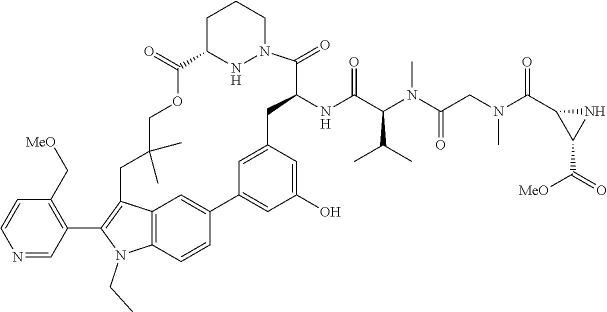 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C(=O)OC)C(=O)N1CCC[C@H](N1)C(=O)OCC(C)(C)C2] |
| C50 | 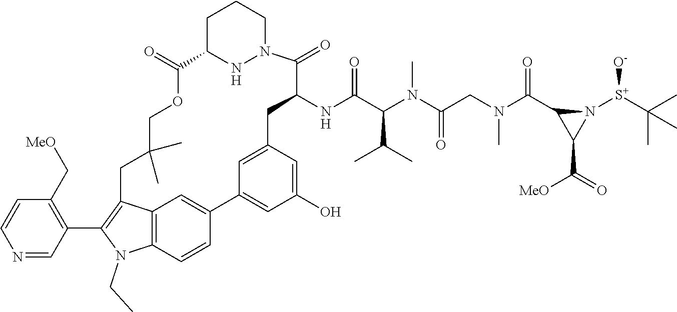 |
| C51 | 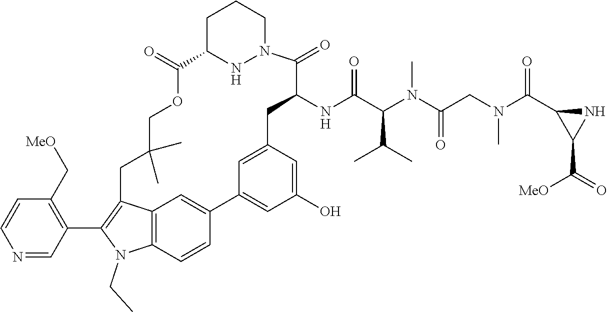 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C(=O)OC)C(=O)N1CCC[C@H](N1)C(=O)OCC(C)(C)C2] |
| C52 | 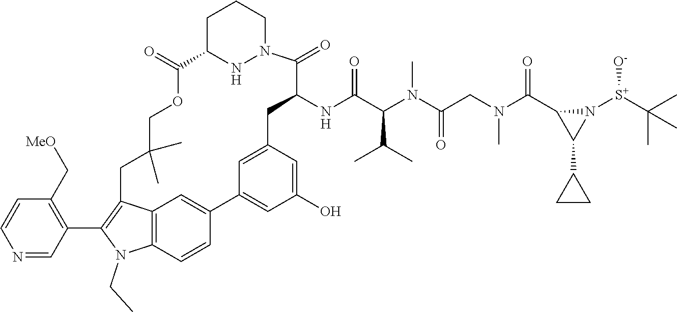 |
| C53 | 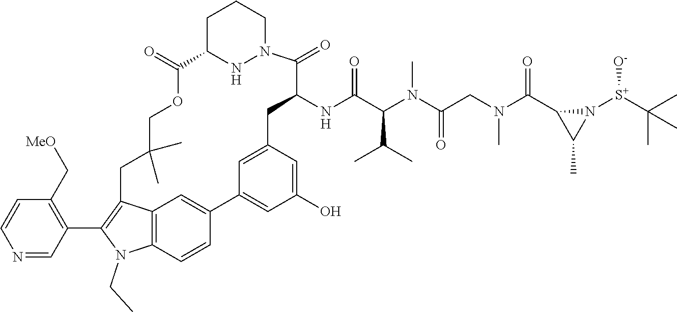 |
| C54 | 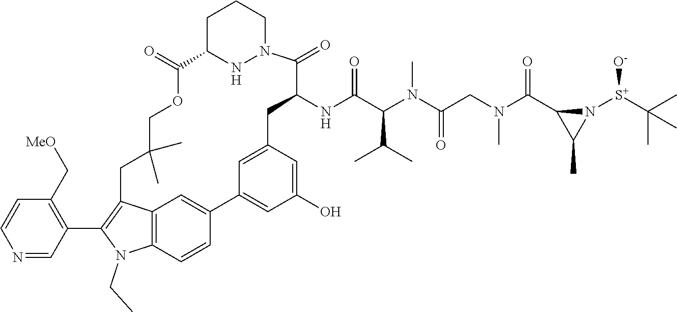 |
| C55 | 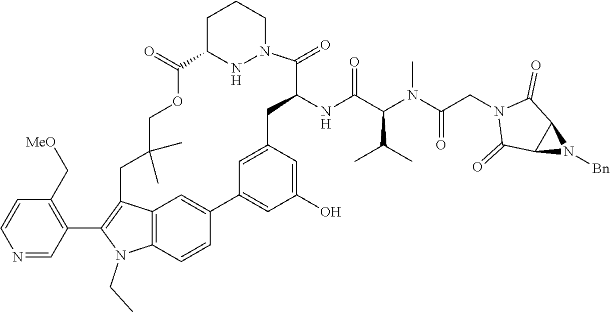 |
| C56* | 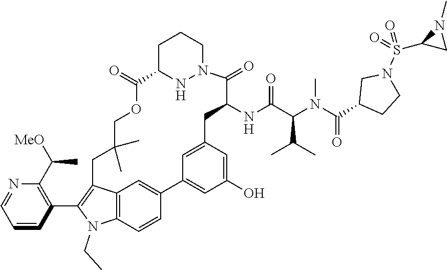 |
| C57* | 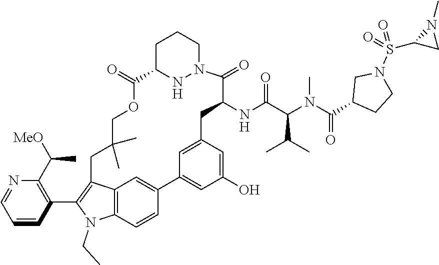 |
| C58 | 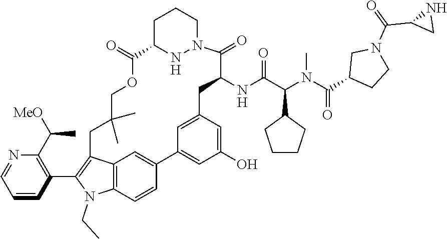 |
| C59 | 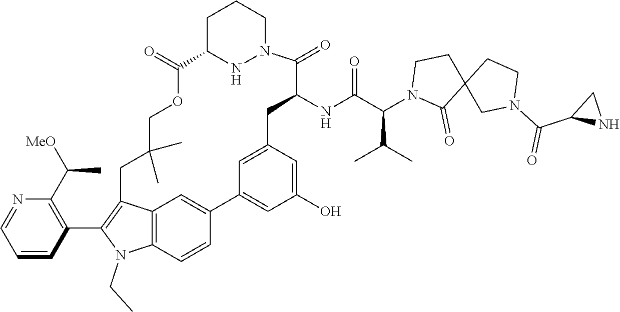 |
| C60 | 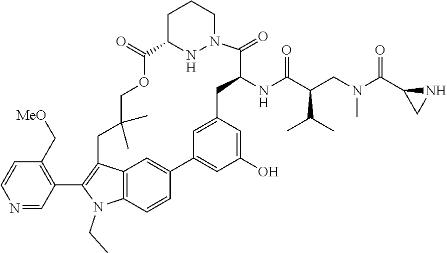 [SMILES: CCn1c(-c2cnccc2COC)c2c3cc(ccc31)-c1cc(O)cc(c1)C[C@H](NC(=O)[C@@H](CN(C)C(=O)[C@@H]1CN1)C(C)C)C(=O)N1CCC[C@H](N1)C(=O)OCC(C)(C)C2] |
| C61 A and B | 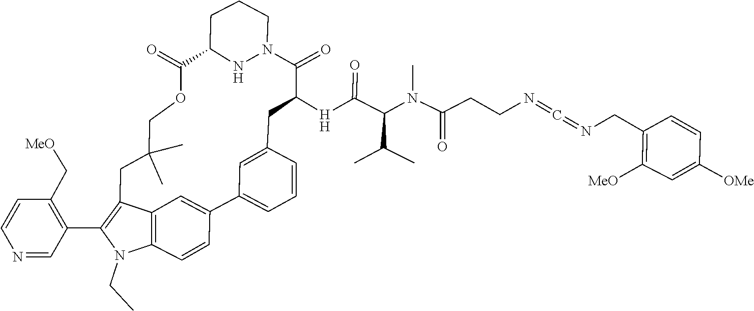 |
| C62 A and B | 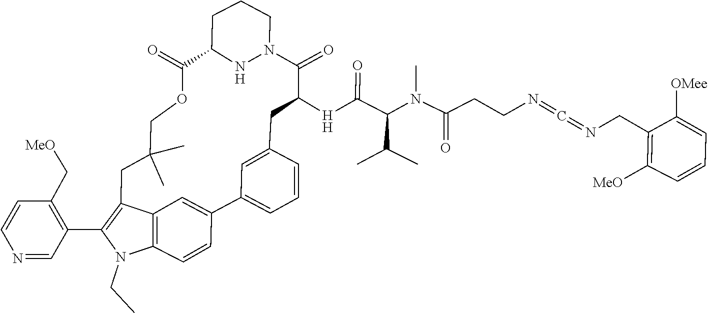 |
| C63 | 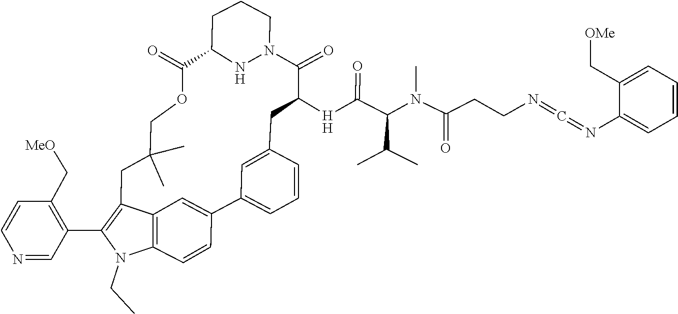 |
| C64 A and B | 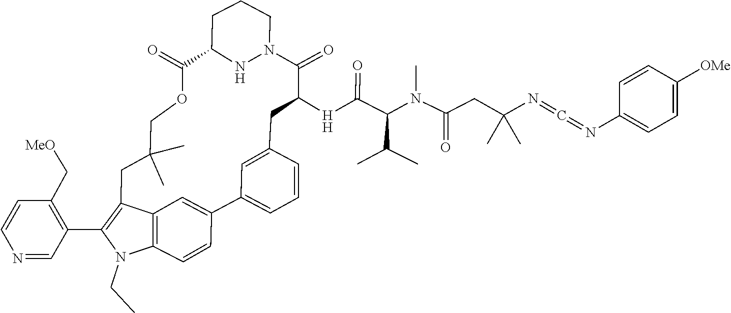 |
| C65 A and B | 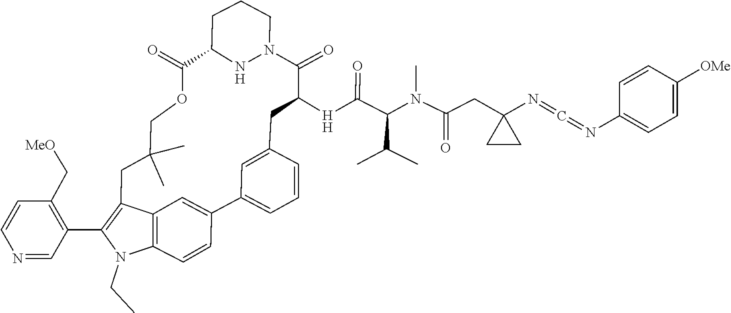 |
| C66 | 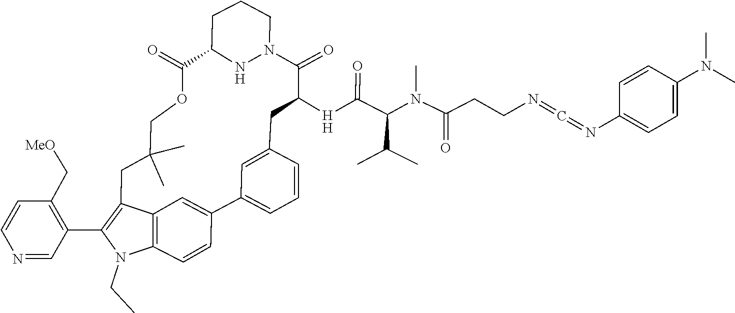 |
| C67 | 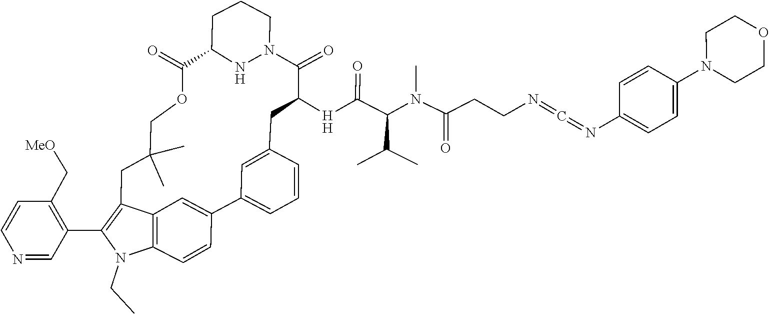 |
| C68 A and B | 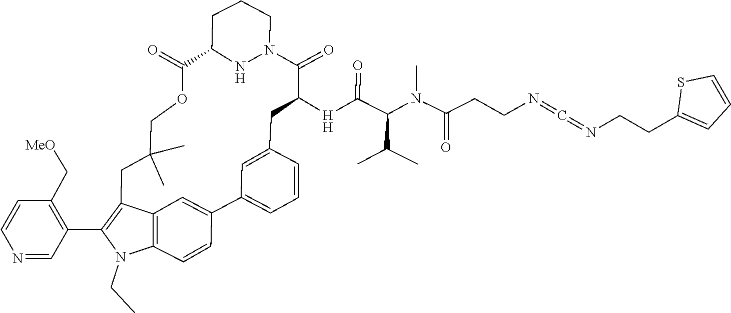 |
| C69 A and B | 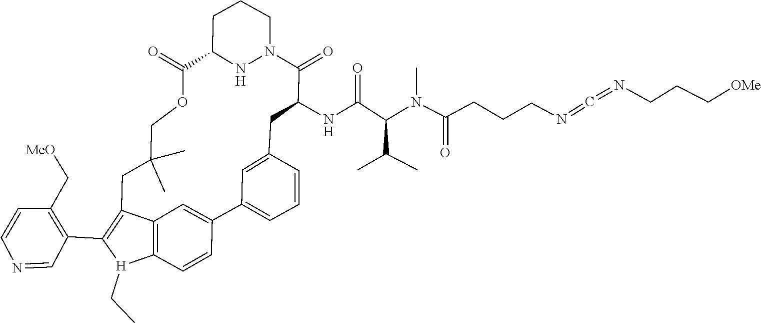 |
| C70 A and B | 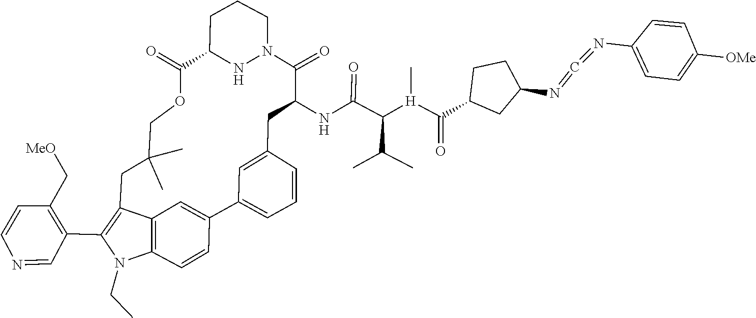 |
| C71 A and B | 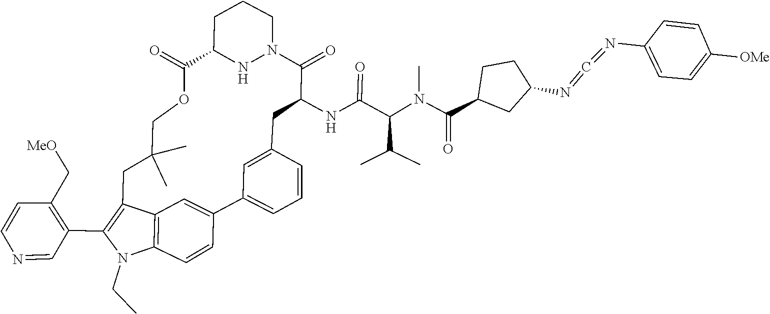 [SMILES: CCn1c(-c2cnccc2COC)c2c3cc(ccc31)-c1cccc(c1)C[C@H](NC(=O)[C@H](C(C)C)N(C)C(=O)[C@H]1CC[C@H](N=C=Nc3ccc(OC)cc3)C1)C(=O)N1CCC[C@H](N1)C(=O)OCC(C)(C)C2] |
| C72 A and B | 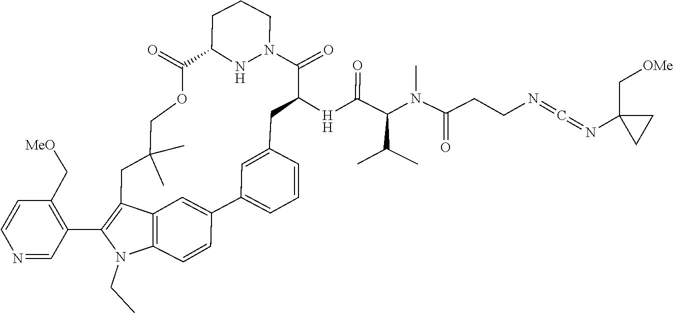 |
| C73 | 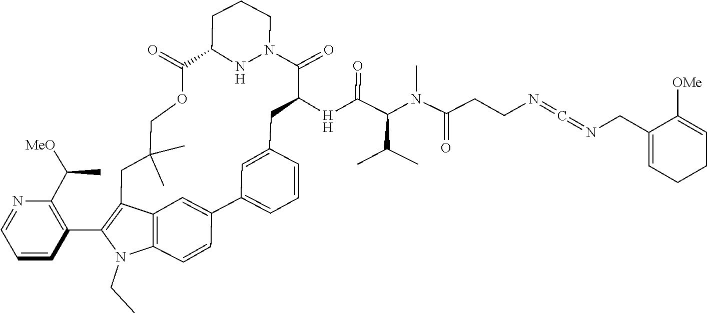 |
| C74 | 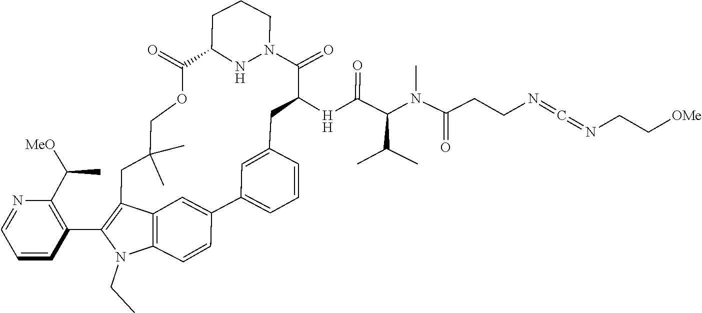 |
| C75 | 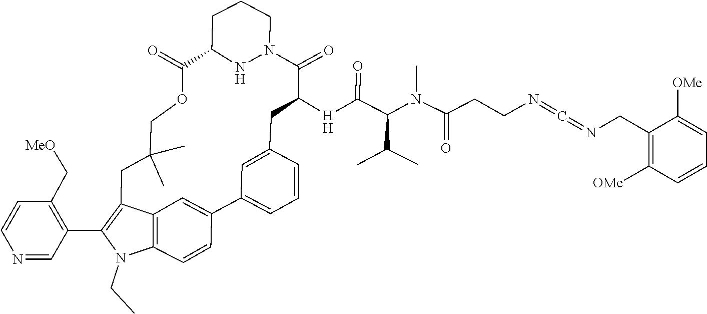 |
| C76 | 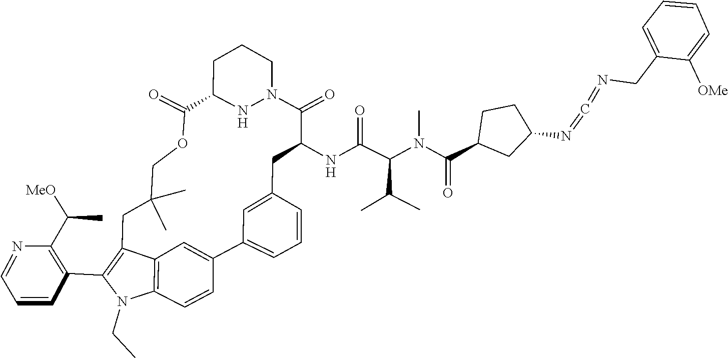 |
| C77 | 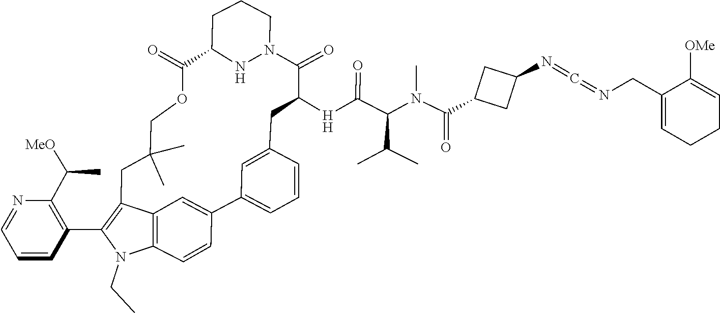 |
| C78 | 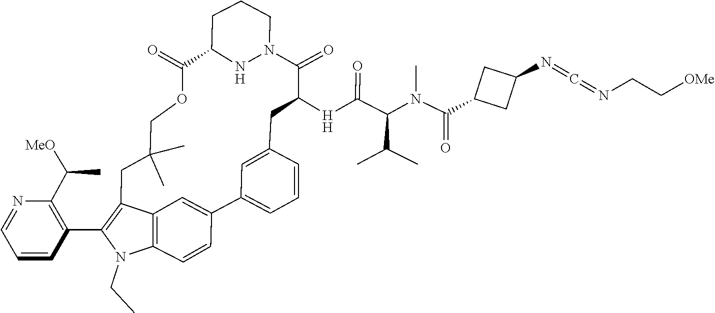 |
| C79 A and B | 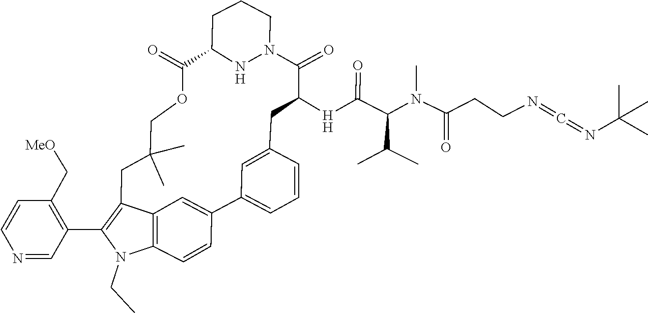 |
| C80 | 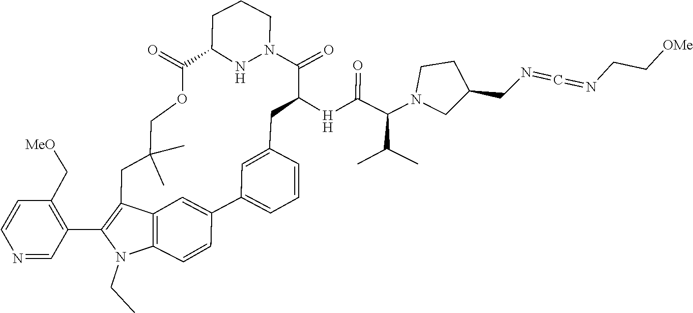 |
| C81 | 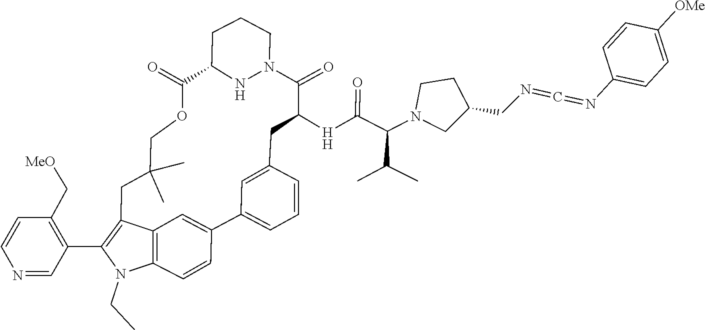 |
| C82 A and B | 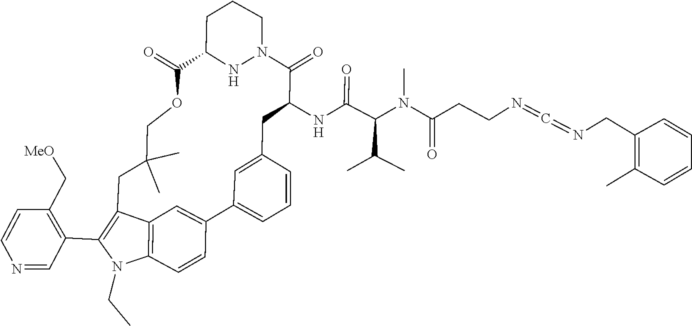 |
| C83 | 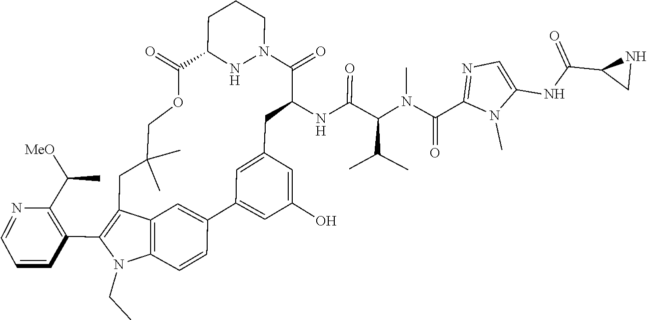 |
| C84 | 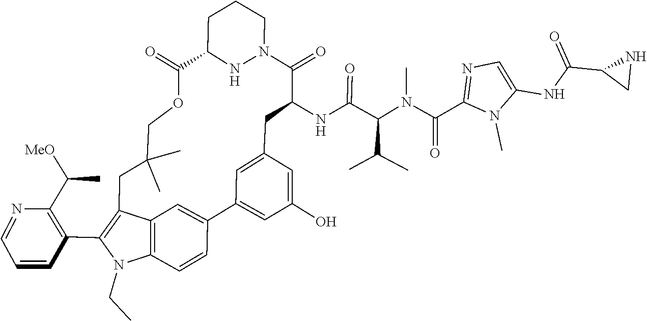 |
| C85 | 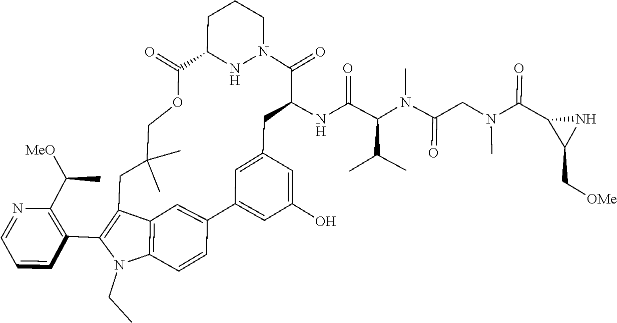 |
| C86 | 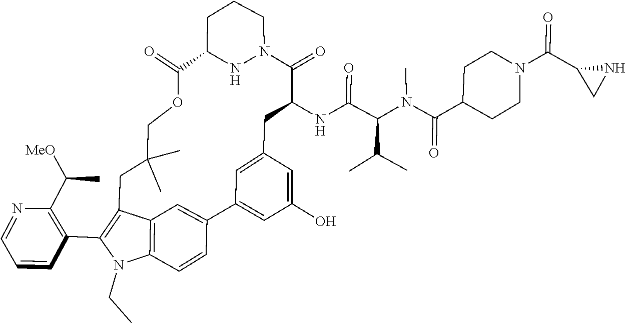 |
| C87 | 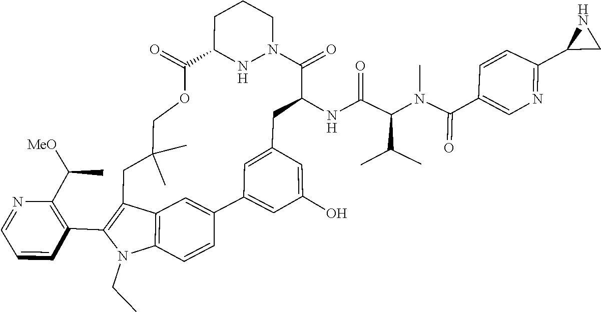 |
| C88 | 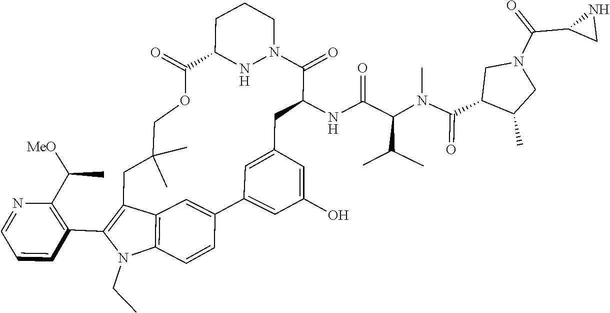 |
| C89 | 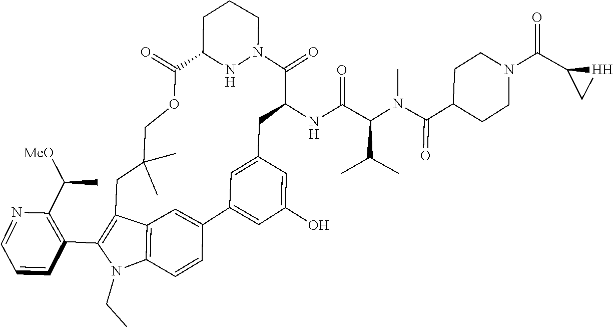 |
| C90 | 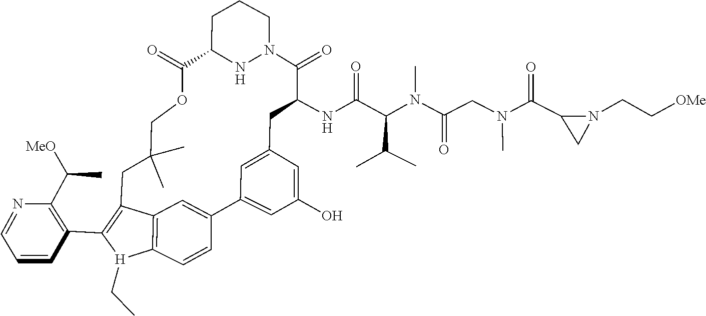 |
| C91* | 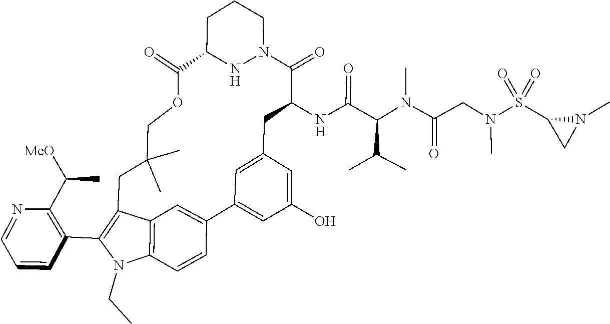 |
| C92* | 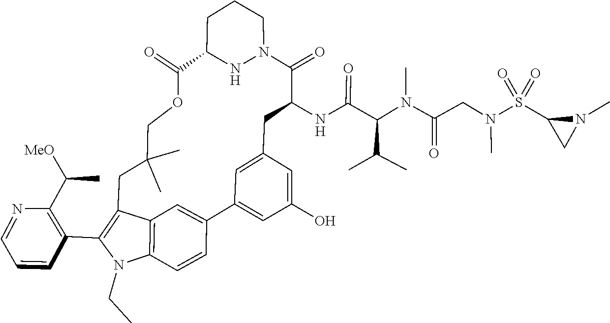 |
| C93 | 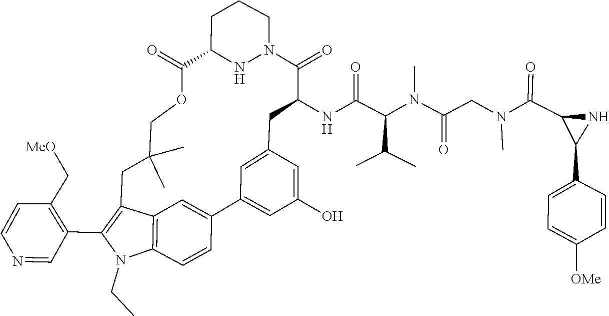 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1c1ccc(OC)cc1)C(=O)N1CCC[C@H](N1)C(=O)OCC(C)(C)C2] |
| C94 | 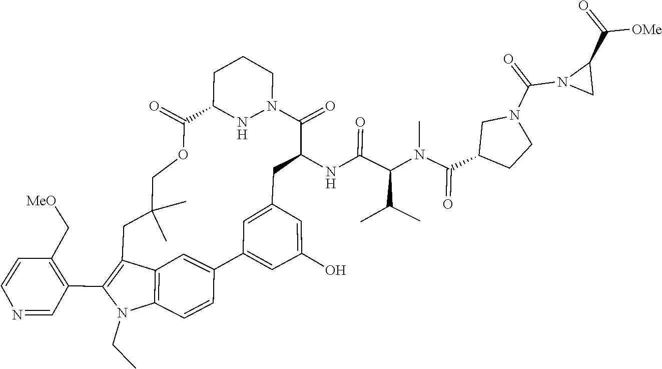 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3C[C@@H]3C(=O)OC)C1)C(=O)N1CCC[C@H](N1)C(=O)OCC(C)(C)C2] |
| C95 | 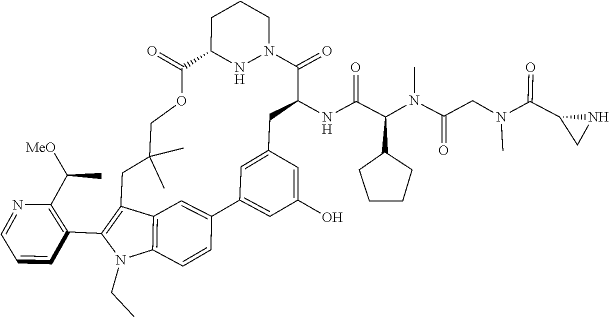 |
| C96 | 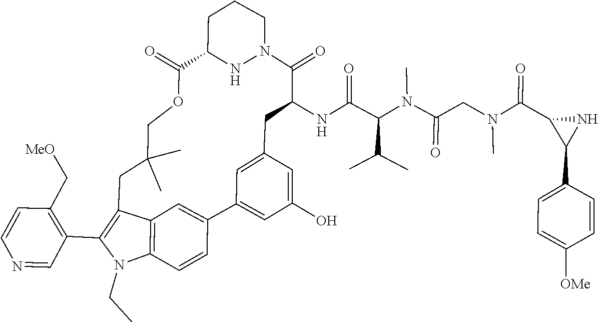 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1c1ccc(OC)cc1)C(=O)N1CCC[C@H](N1)C(=O)OCC(C)(C)C2] |
| C97 | 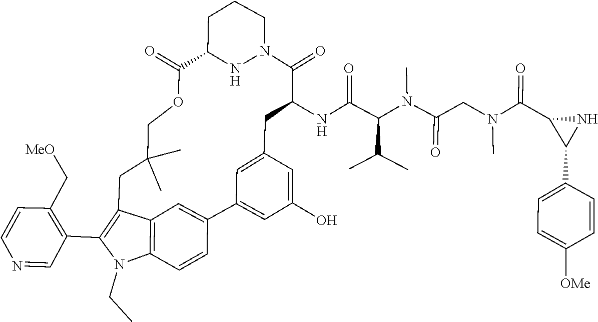 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1c1ccc(OC)cc1)C(=O)N1CCC[C@H](N1)C(=O)OCC(C)(C)C2] |
| C98 | 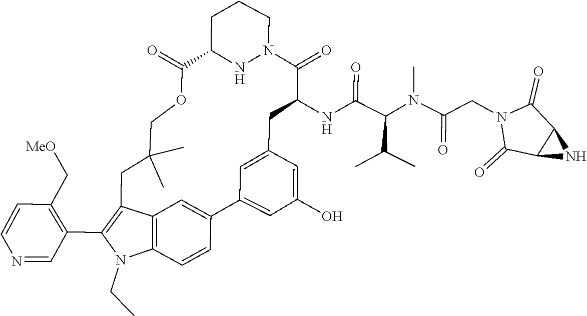 [SMILES: CCn1c(-c2cnccc2COC)c2c3cc(ccc31)-c1cc(O)cc(c1)C[C@H](NC(=O)[C@H](C(C)C)N(C)C(=O)CN1C(=O)[C@H]3N[C@H]3C1=O)C(=O)N1CCC[C@H](N1)C(=O)OCC(C)(C)C2] |
| C99 | 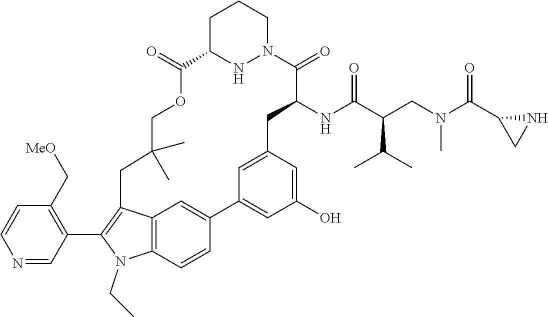 [SMILES: CCn1c(-c2cnccc2COC)c2c3cc(ccc31)-c1cc(O)cc(c1)C[C@H](NC(=O)[C@@H](CN(C)C(=O)[C@H]1CN1)C(C)C)C(=O)N1CCC[C@H](N1)C(=O)OCC(C)(C)C2] |
| C100 | 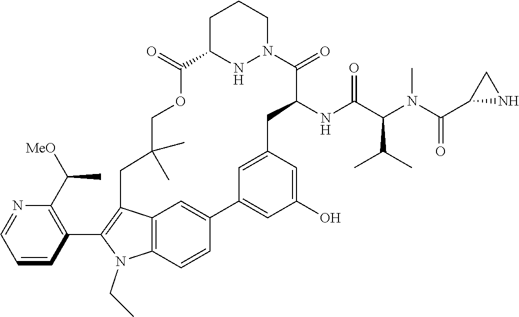 |
| C101 | 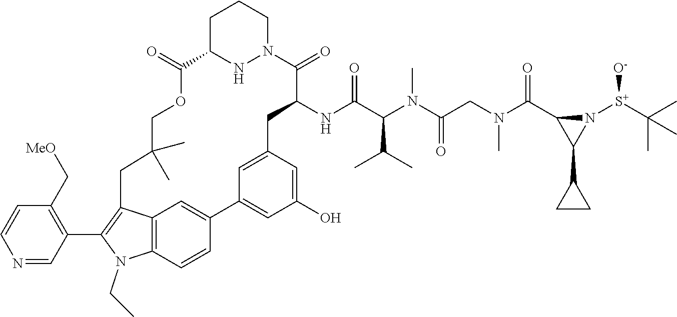 |
| C102 | 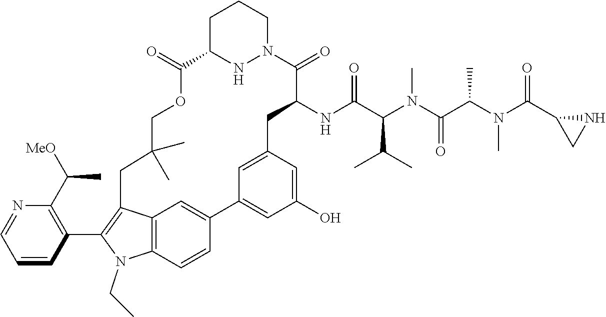 |
| C103 | 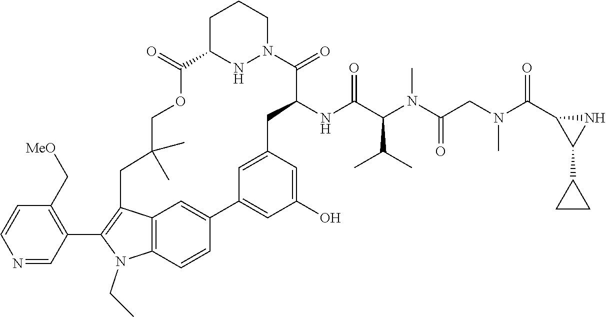 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C1CC1)C(=O)N1CCC[C@H](N1)C(=O)OCC(C)(C)C2] |
| C104 | 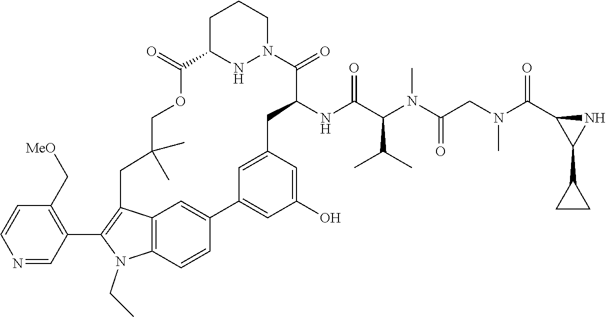 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C1CC1)C(=O)N1CCC[C@H](N1)C(=O)OCC(C)(C)C2] |
| C105 | 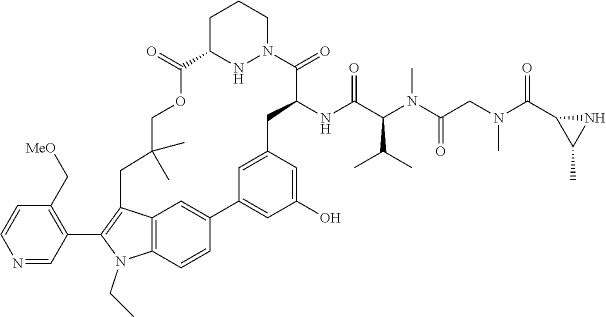 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1C)C(=O)N1CCC[C@H](N1)C(=O)OCC(C)(C)C2] |
| C106 | 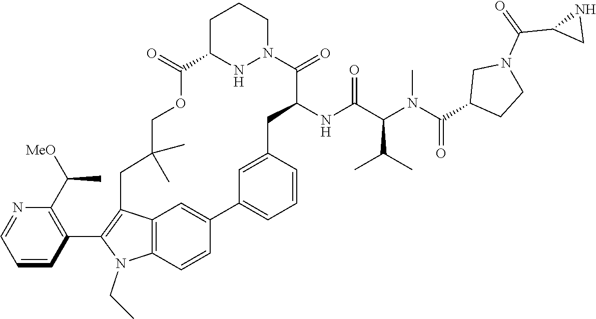 |
| C107 | 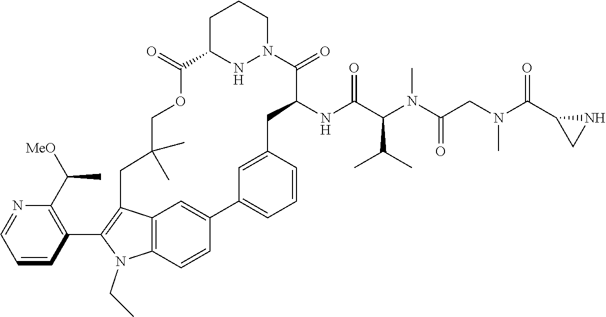 |
| C108* | 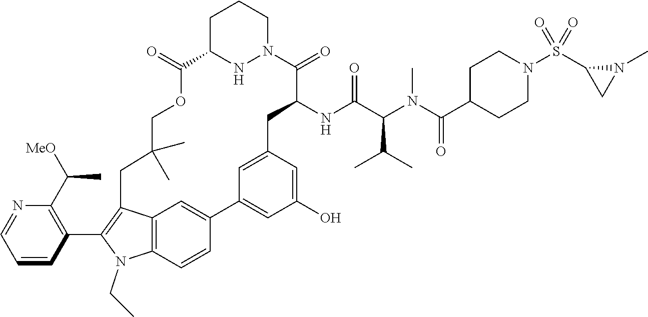 |
| C109 | 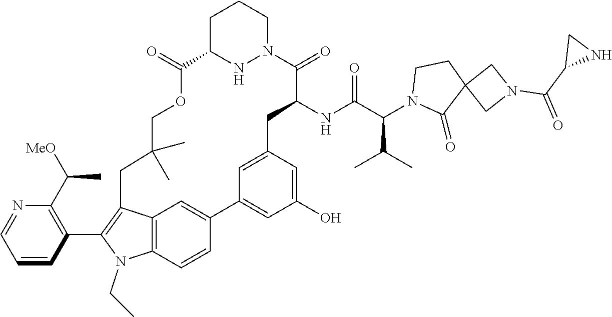 |
| C110 | 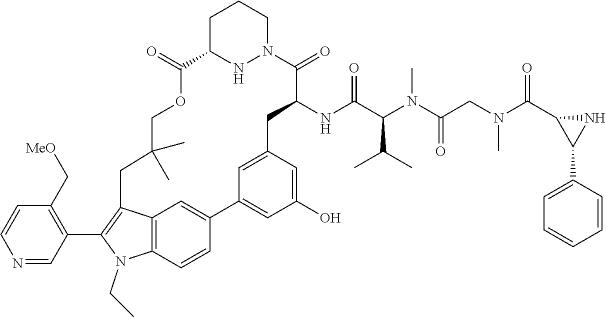 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1c1ccccc1)C(=O)N1CCC[C@H](N1)C(=O)OCC(C)(C)C2] |
| C111 | 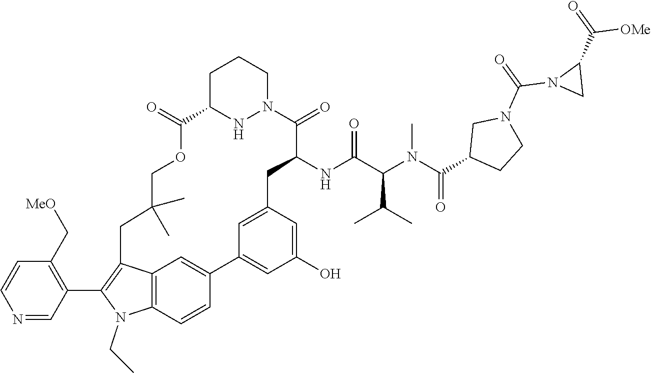 [SMILES: CCn1c(-c2cnccc2COC)c2c3cc(ccc31)-c1cc(O)cc(c1)C[C@H](NC(=O)[C@H](C(C)C)N(C)C(=O)[C@H]1CCN(C(=O)N3C[C@H]3C(=O)OC)C1)C(=O)N1CCC[C@H](N1)C(=O)OCC(C)(C)C2] |
| C112 | 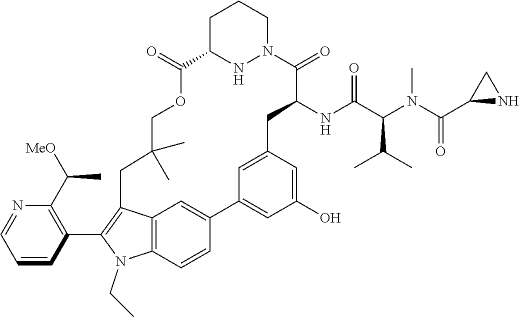 |
| C113 | 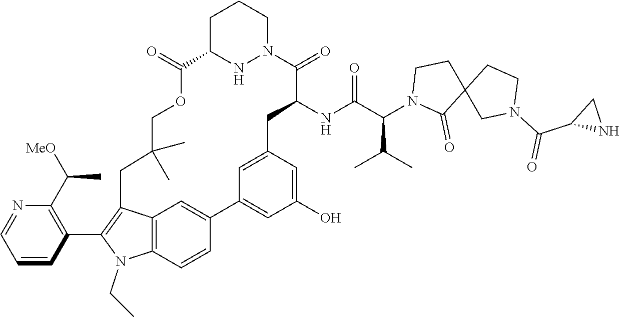 |
| C114 | 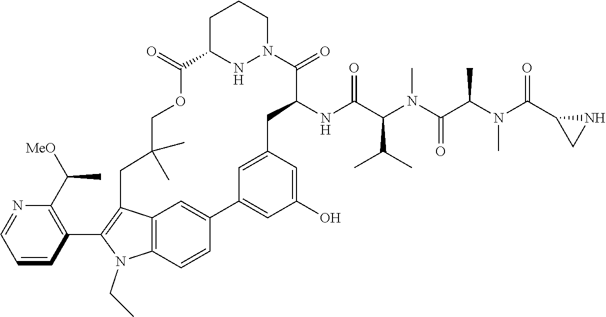 |
| C115 | 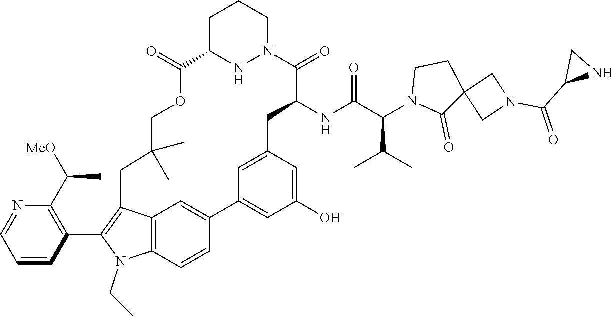 |
| C116 | 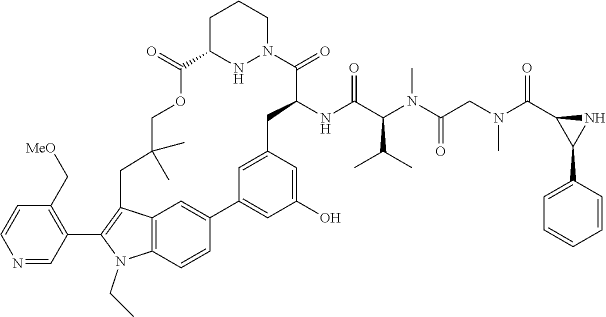 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1c1ccccc1)C(=O)N1CCC[C@H](N1)C(=O)OCC(C)(C)C2] |
| C117* | 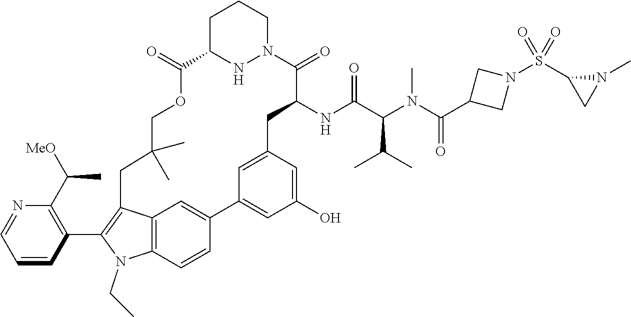 |
| C118* | 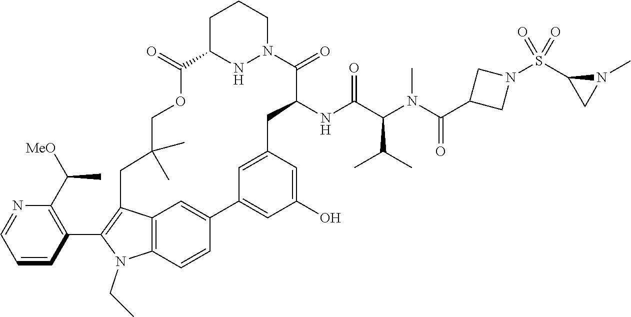 |
| C119* | 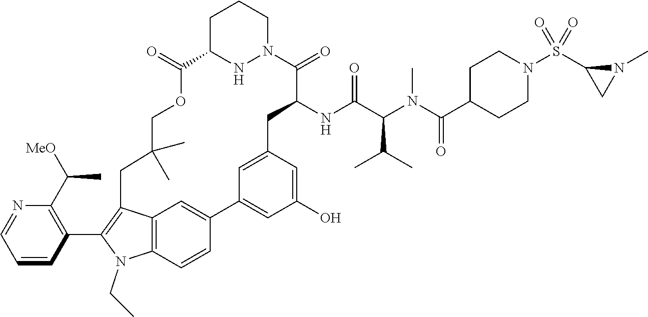 |
| C120 | 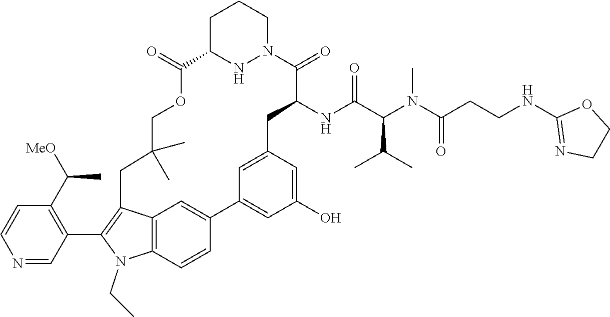 [SMILES: CCn1c(-c2cnccc2[C@H](C)OC)c2c3cc(ccc31)-c1cc(O)cc(c1)C[C@H](NC(=O)[C@H](C(C)C)N(C)C(=O)CCNC1=NCCO1)C(=O)N1CCC[C@H](N1)C(=O)OCC(C)(C)C2] |
| C121 | 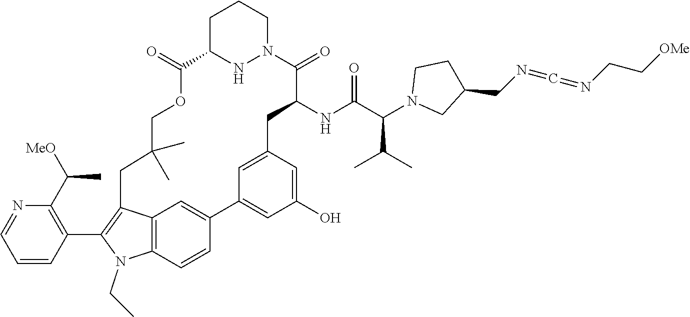 [SMILES: CCn1c(-c2cccnc2[C@H](C)OC)c2c3cc(ccc31)-c1cc(O)cc(c1)C[C@H](NC(=O)[C@H](C(C)C)N1CC[C@@H](CN=C=NCCOC)C1)C(=O)N1CCC[C@H](N1)C(=O)OCC(C)(C)C2] |
| C122 | 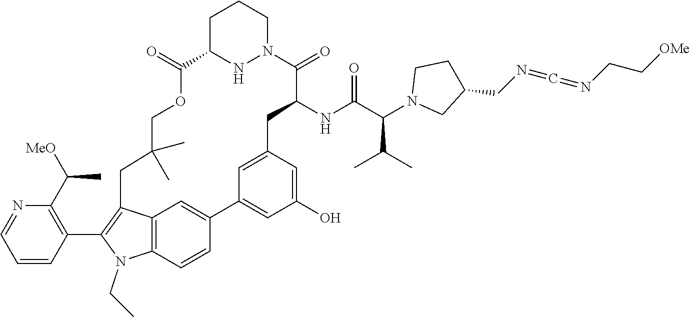 [SMILES: CCn1c(-c2cccnc2[C@H](C)OC)c2c3cc(ccc31)-c1cc(O)cc(c1)C[C@H](NC(=O)[C@H](C(C)C)N1CC[C@H](CN=C=NCCOC)C1)C(=O)N1CCC[C@H](N1)C(=O)OCC(C)(C)C2] |
| C123 | 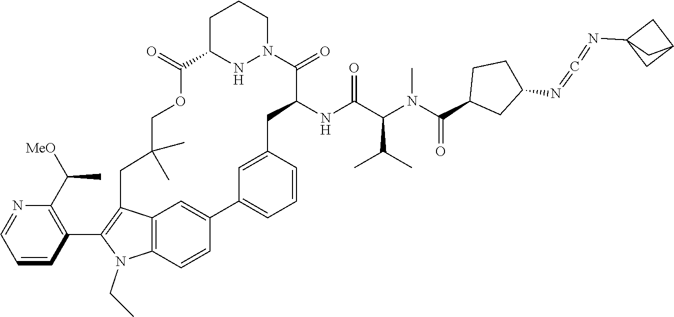 |
| C124 | 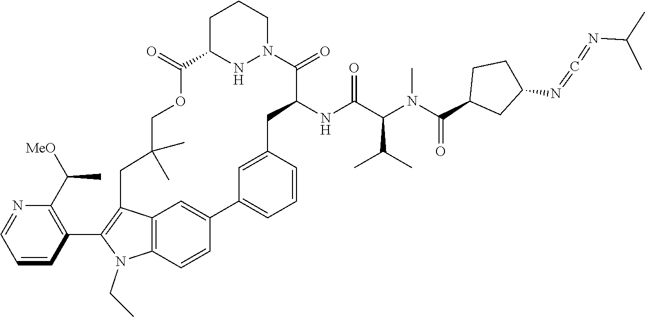 |
| C125 | 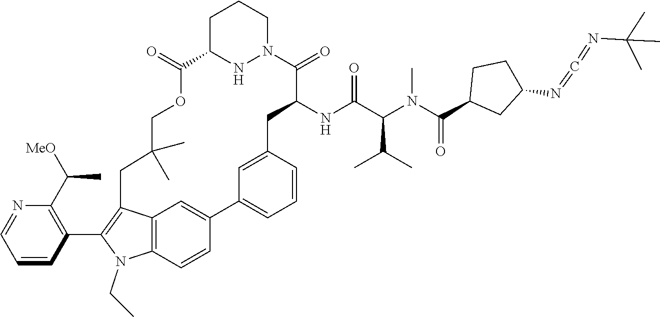 |
| C126 | 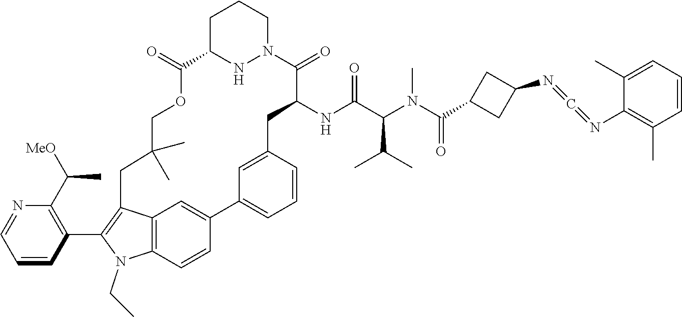 |
| C127 | 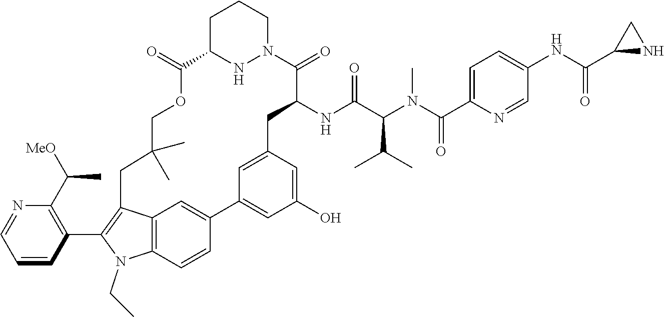 |
| C128 | 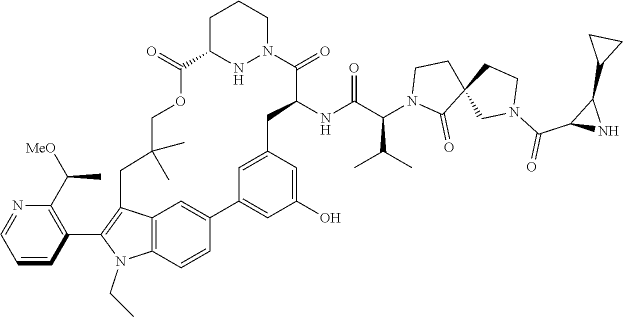 |
| C129 | 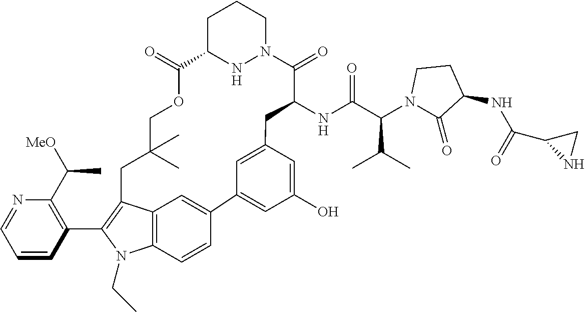 |
| C130 | 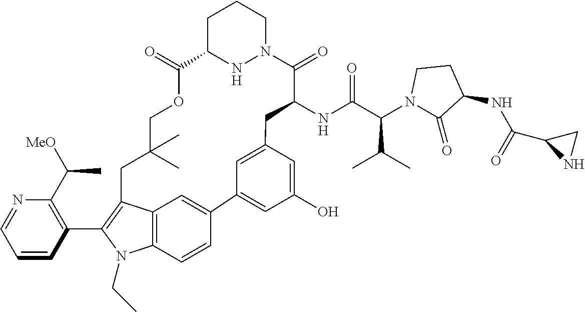 |
| C131 | 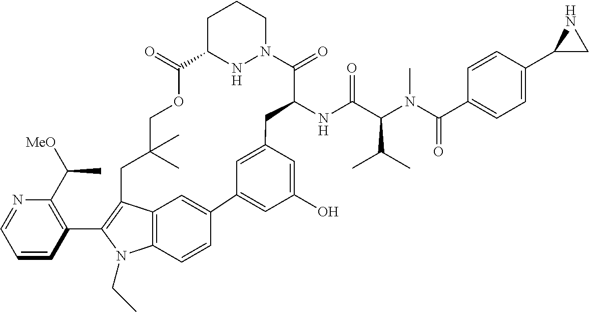 |
| C132 | 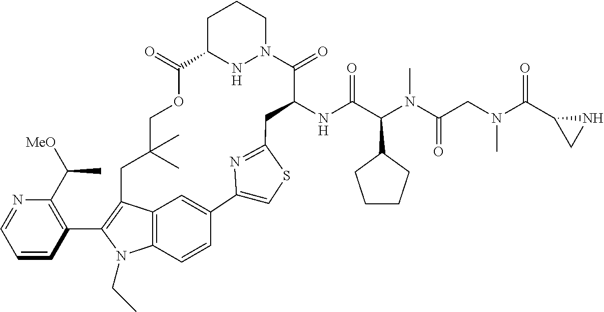 |
| C133 | 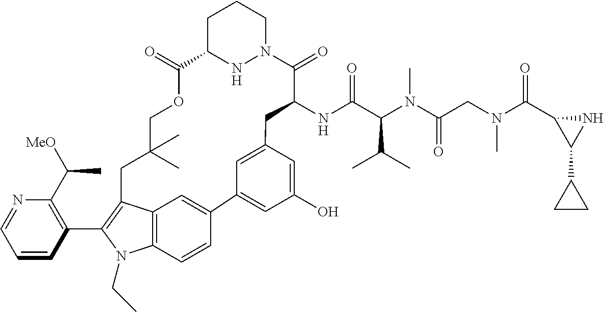 |
| C134 | 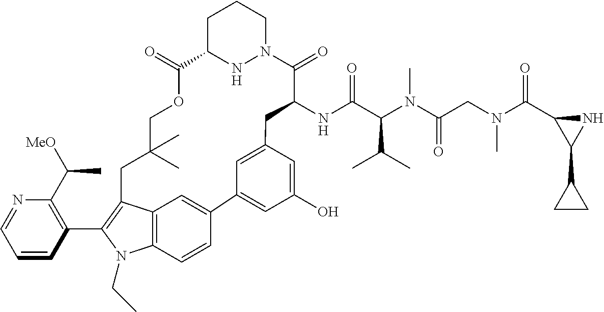 |
| C135 | 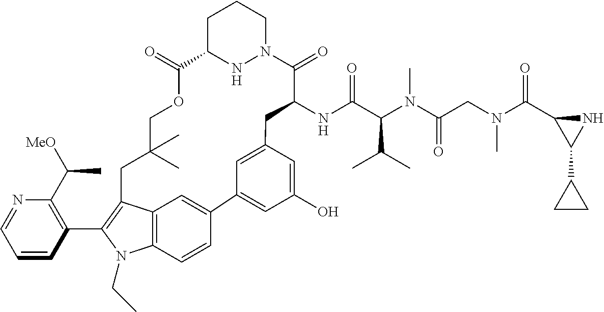 |
| C136 | 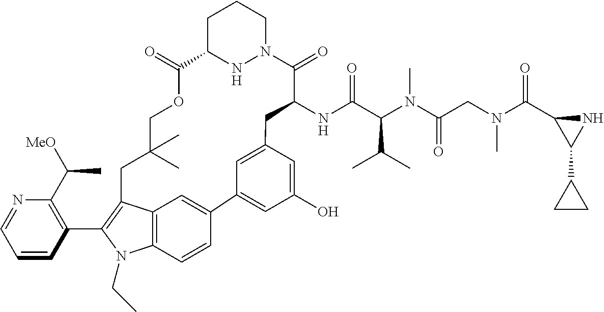 |
| C137 | 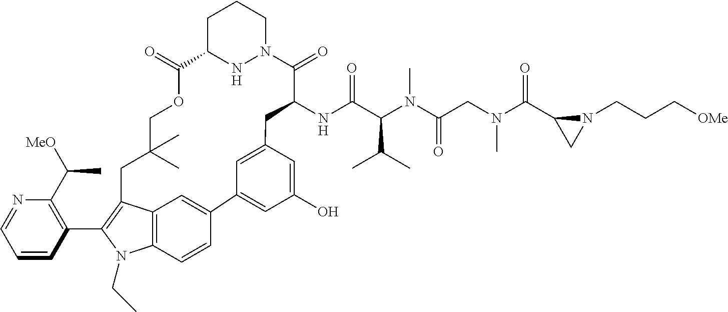 |
| C138 | 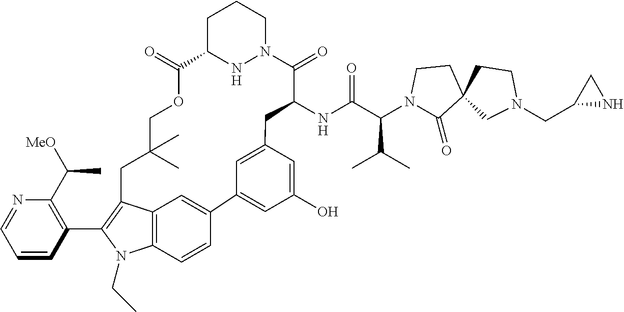 |
| C139 | 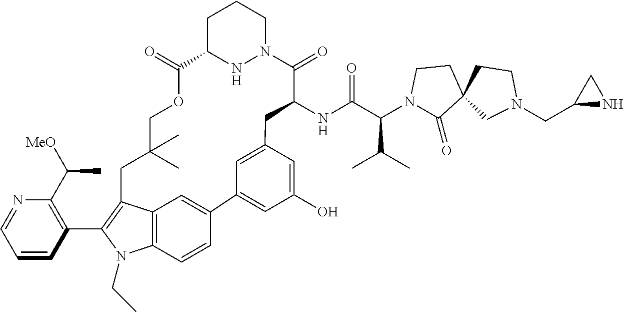 |
| C140 | 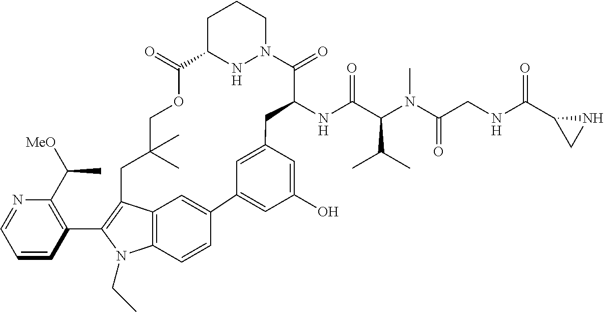 |
| C141 | 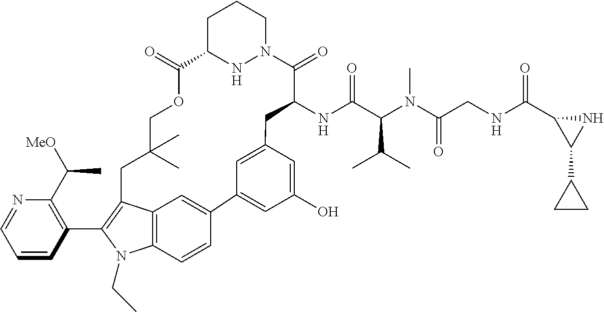 |
| C142 | 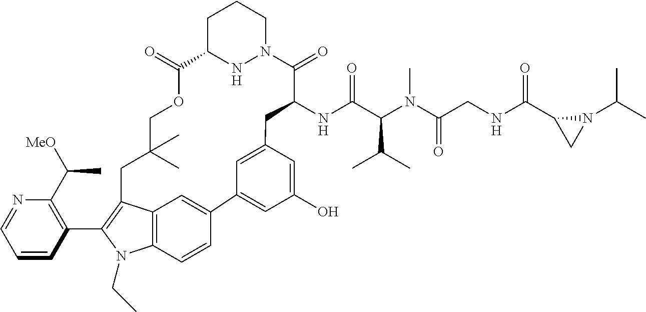 |
| C143 | 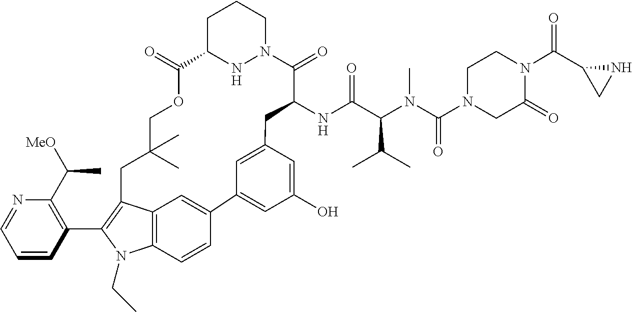 |
| C144 | 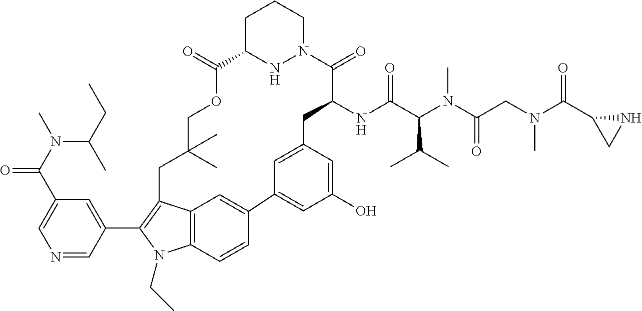 [SMILES: CCC(C)N(C)C(=O)c1cncc(-c2c3c4cc(ccc4n2CC)-c2cc(O)cc(c2)C[C@H](NC(=O)[C@H](C(C)C)N(C)C(=O)CN(C)C(=O)[C@H]2CN2)C(=O)N2CCC[C@H](N2)C(=O)OCC(C)(C)C3)c1] |
| C145 | 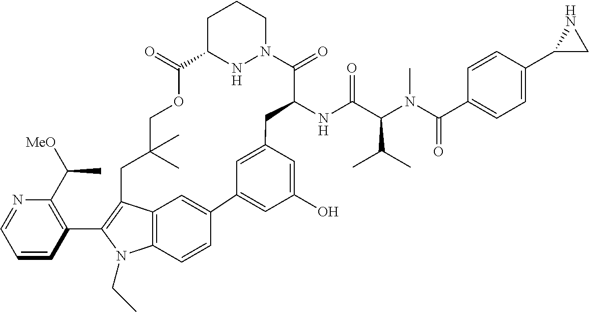 |
| C146 | 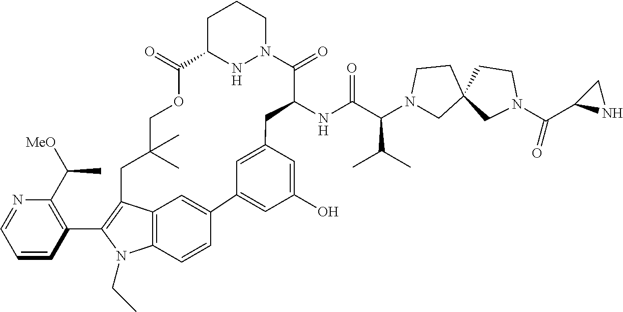 |
| C147 | 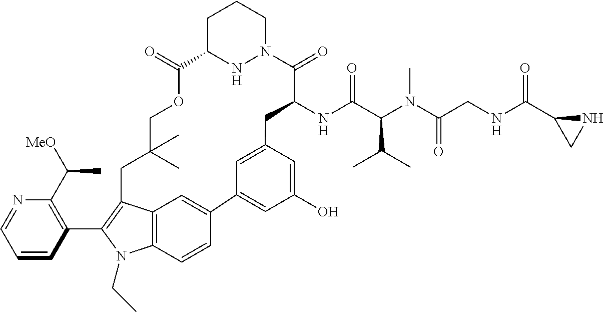 |
| C148 | 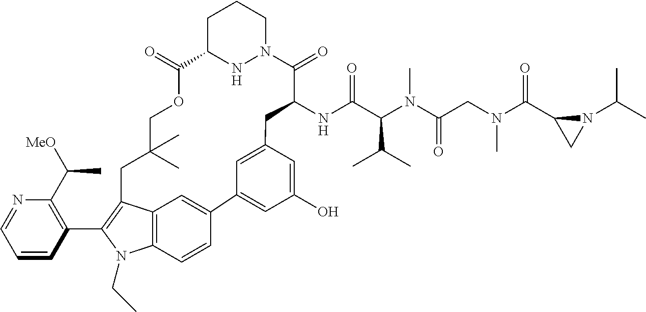 |
| C149 | 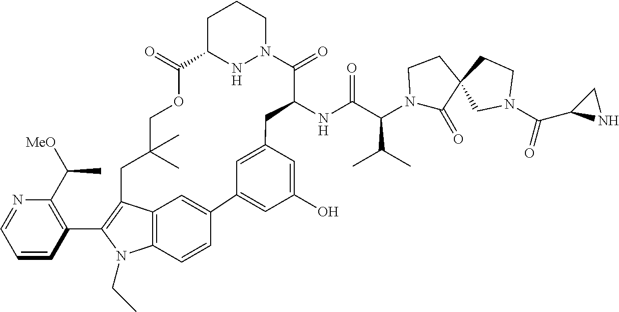 |
| C150 | 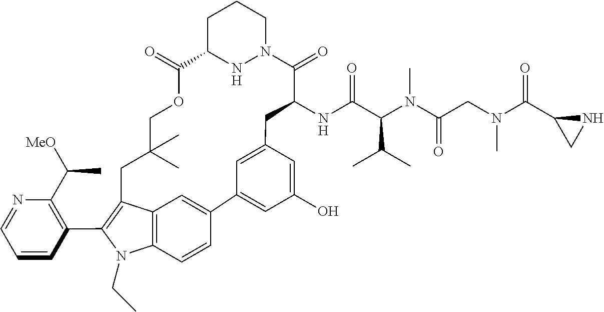 |
| C151 | 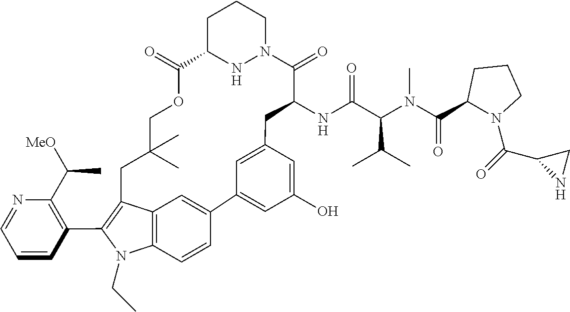 |
| C152 | 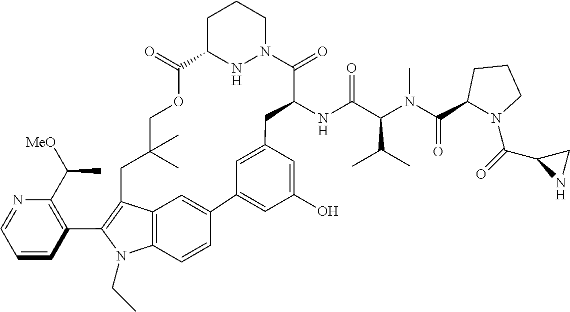 |
| C153 | 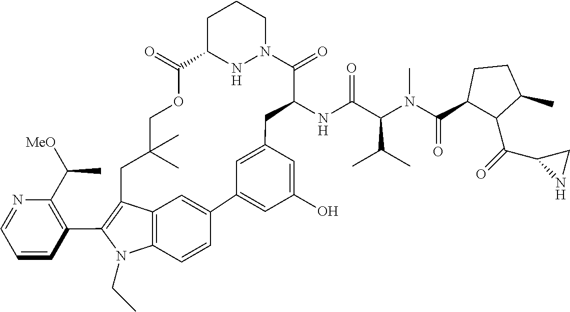 |
| C154 | 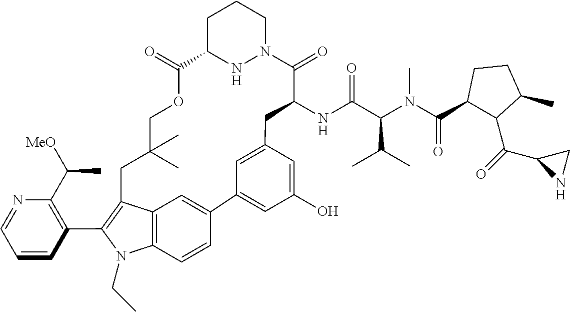 |
| C155 | 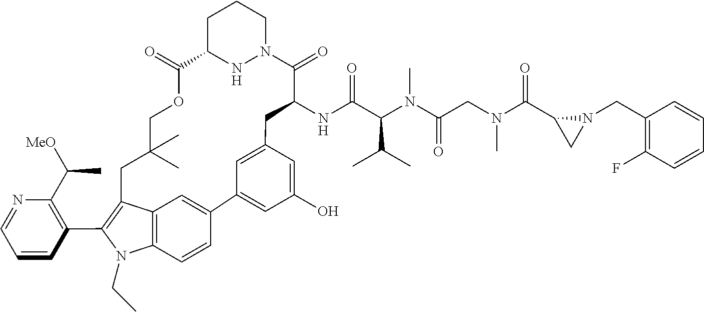 |
| C156 | 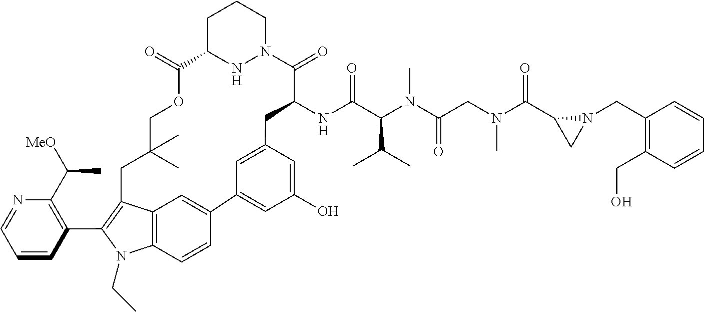 |
| C157 | 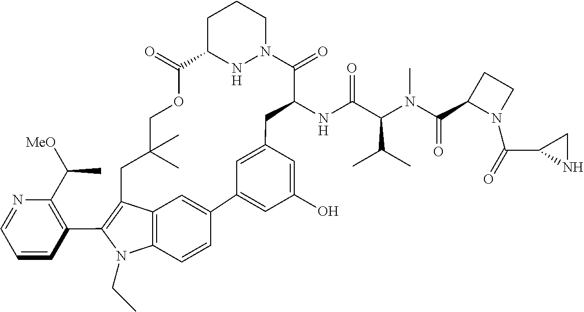 |
| C158 | 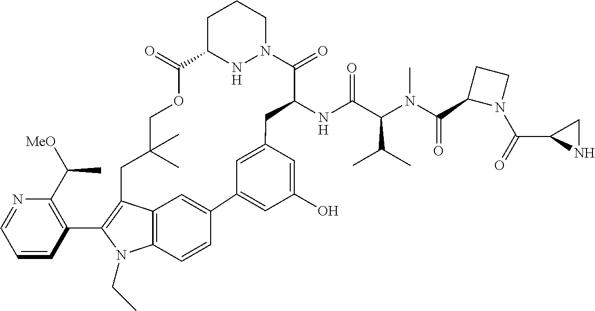 |
| C159 | 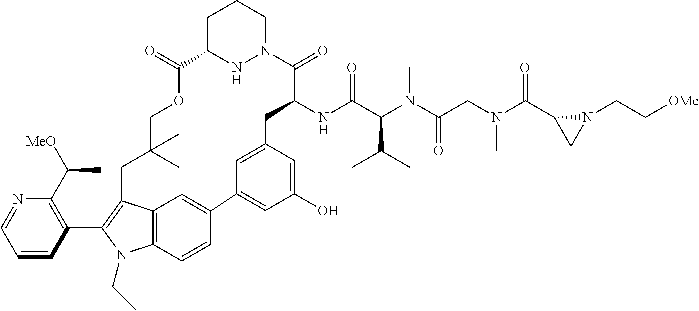 |
| C160 | 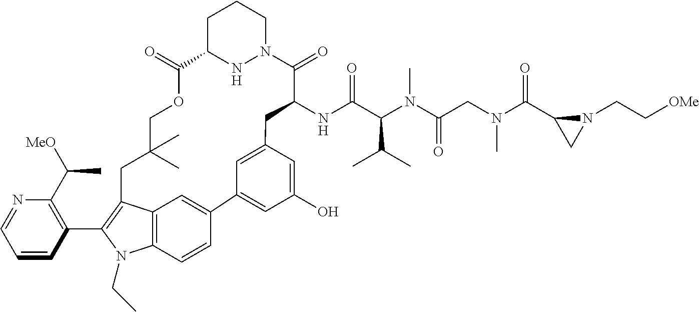 |
| C161 | 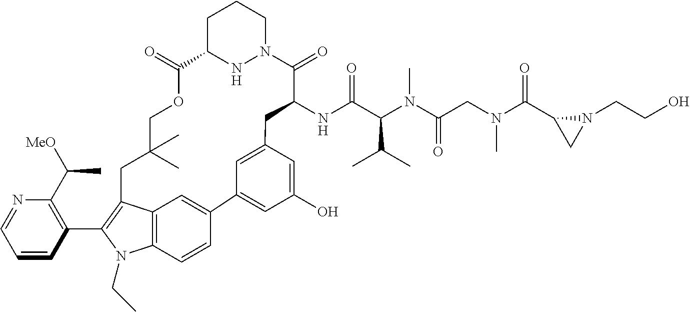 |
| C162 | 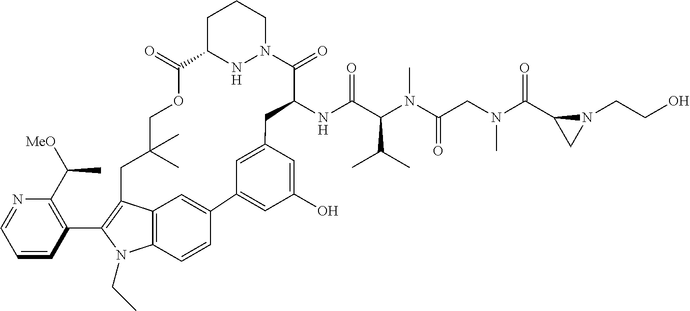 |
| C163 | 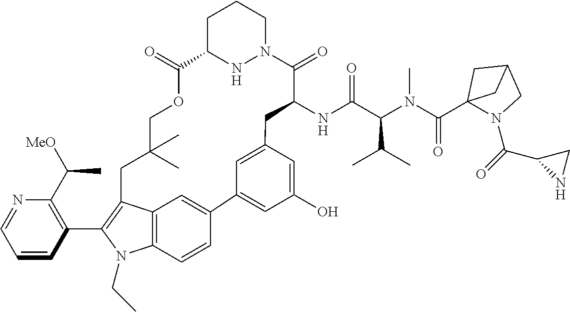 |
| C164 | 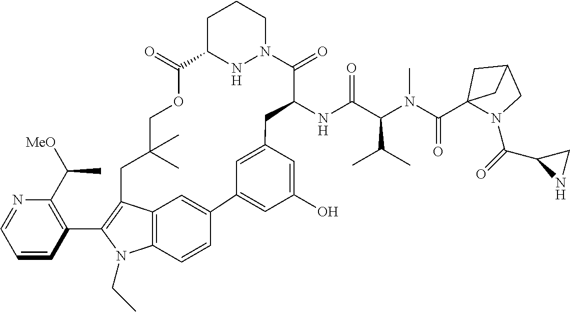 |
| C165 | 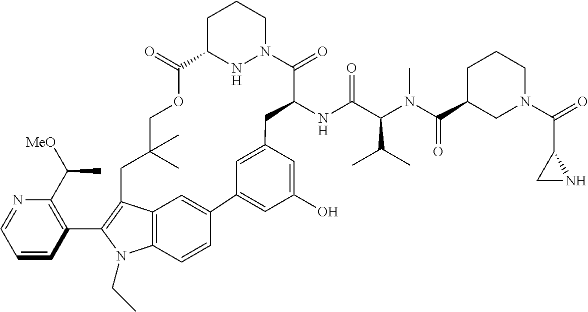 |
| C166 | 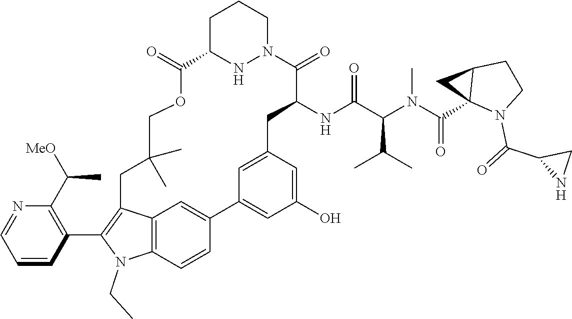 |
| C167 | 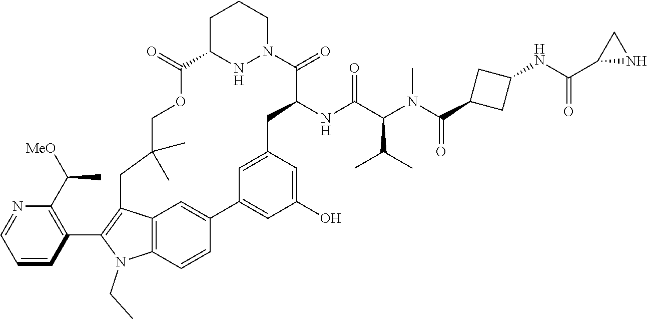 |
| C168 | 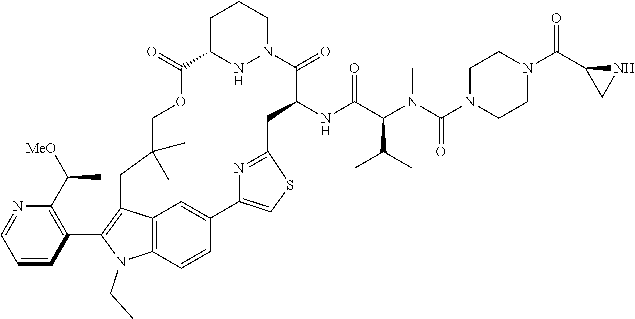 |
| C169 | 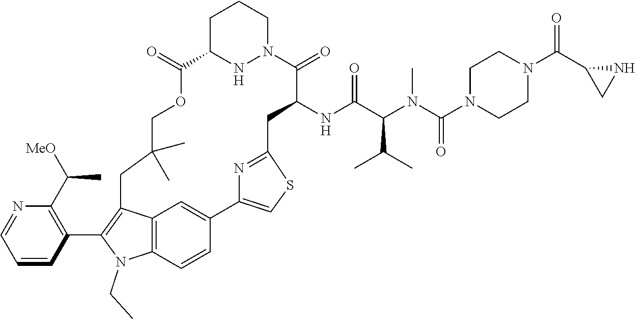 |
| C170 | 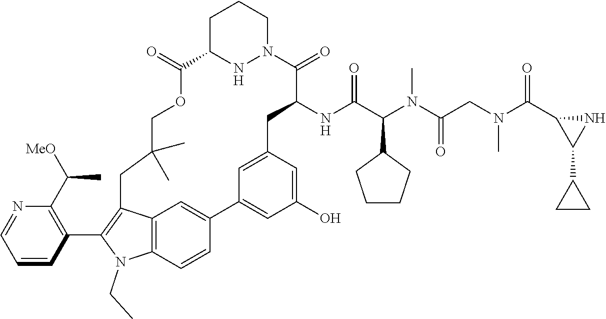 |
| C171 | 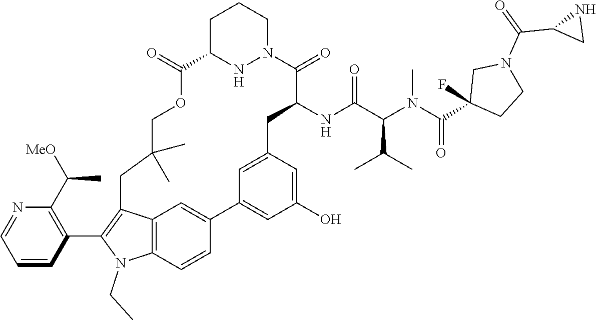 |
| C172 | 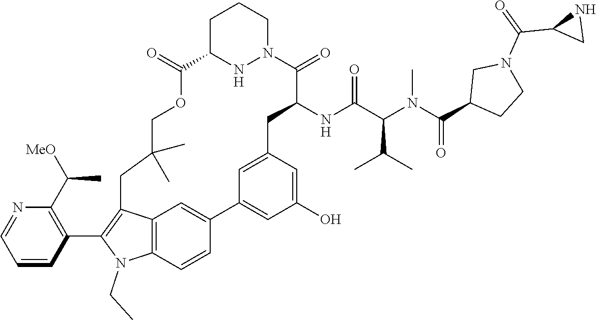 |
| C173 | 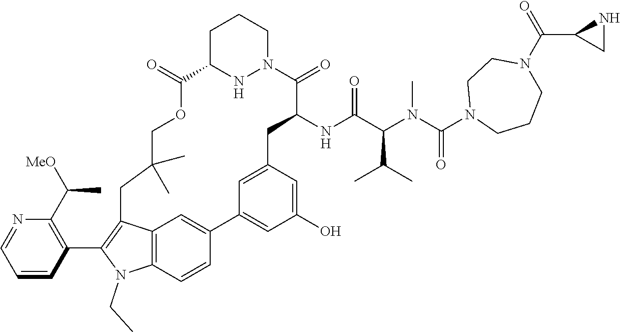 |
| C174 | 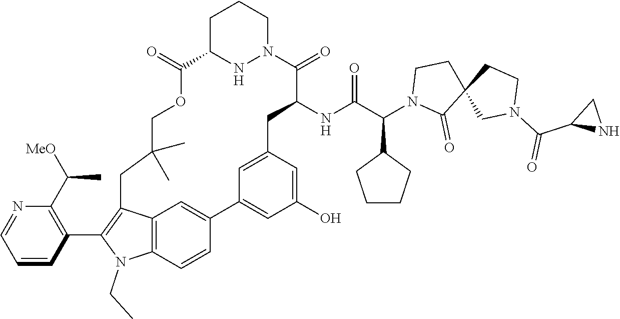 |
| C175 | 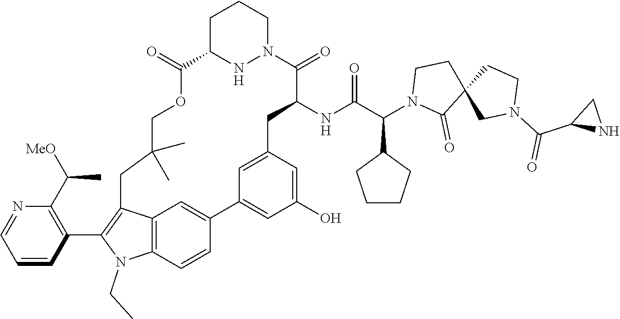 |
| C176 | 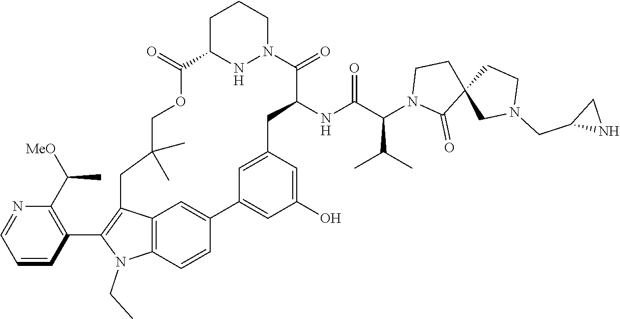 |
| C177 | 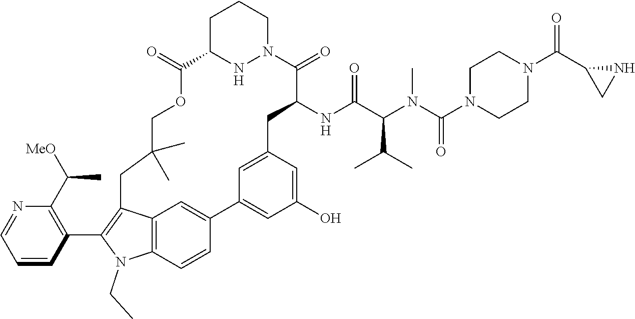 |
| C178 | 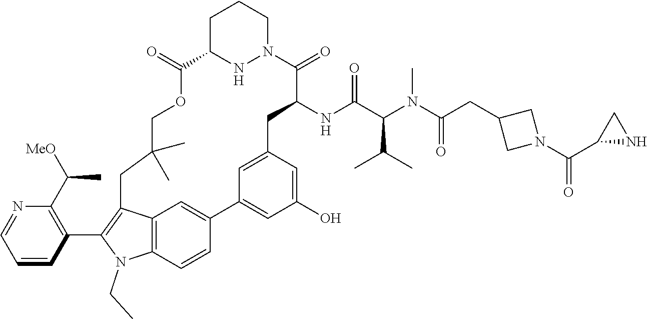 |
| C179 | 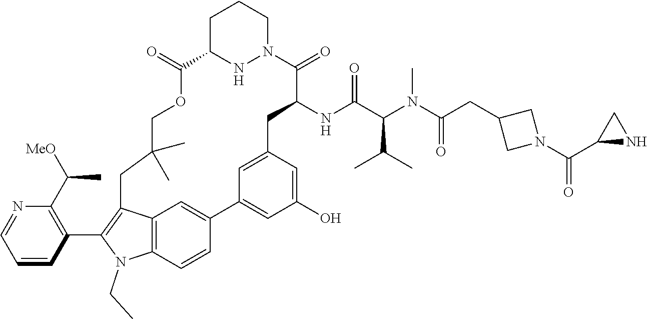 |
| C180 | 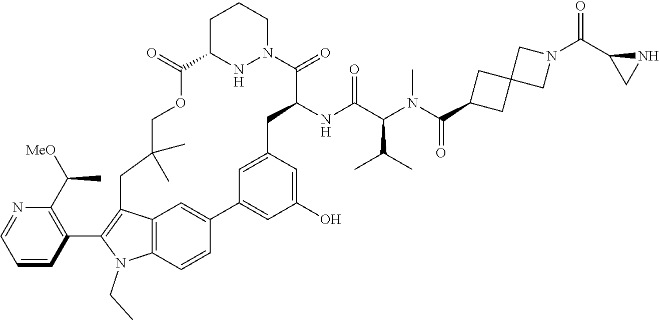 |
| C181 | 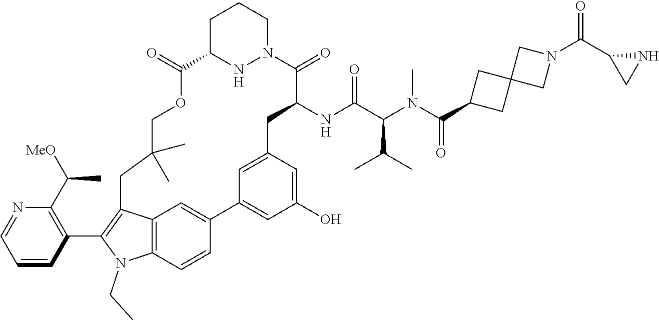 |
| C182 | 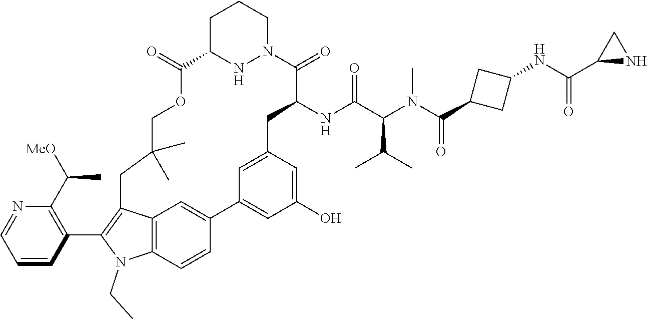 |
| C183 | 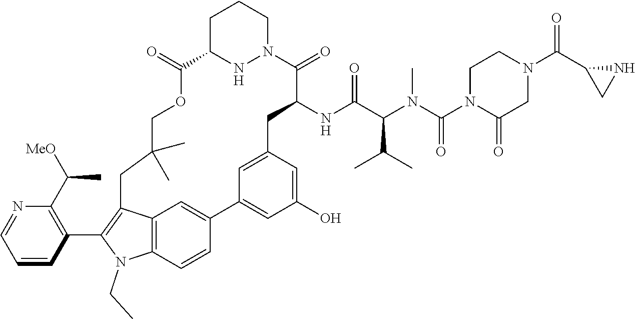 |
| C184 | 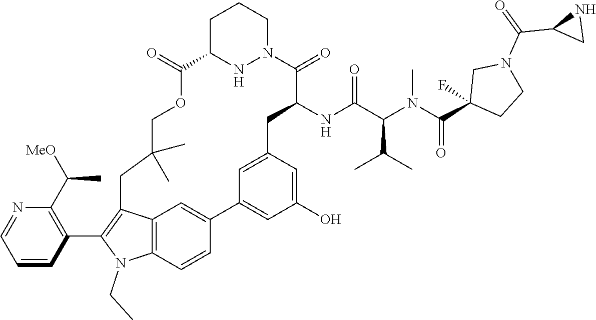 |
| C185 | 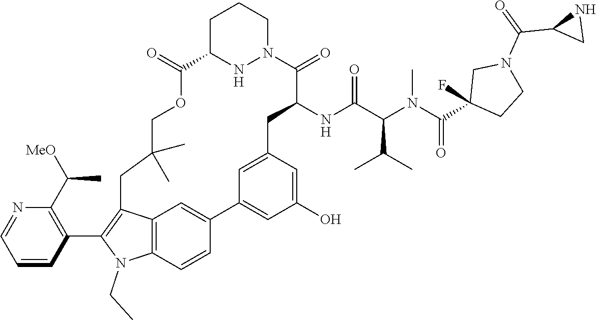 |
| C186 | 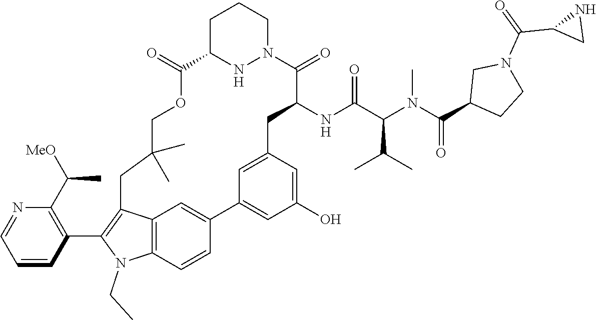 |
| C187 | 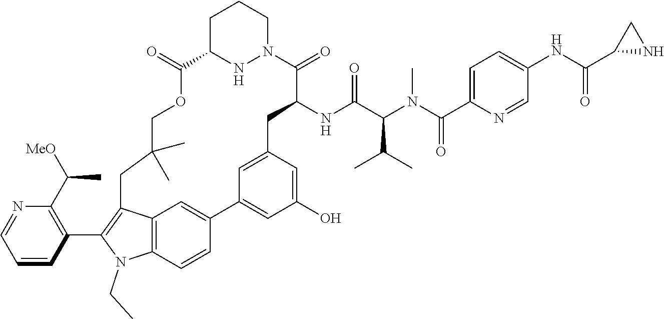 |
| C188 | 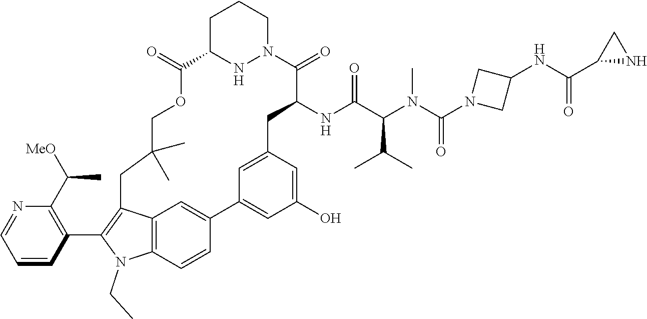 |
| C189 | 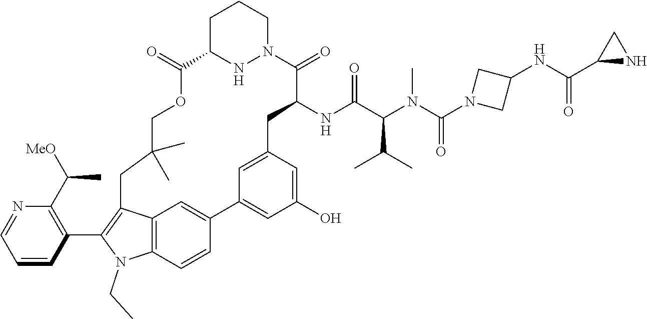 |
| C190 | 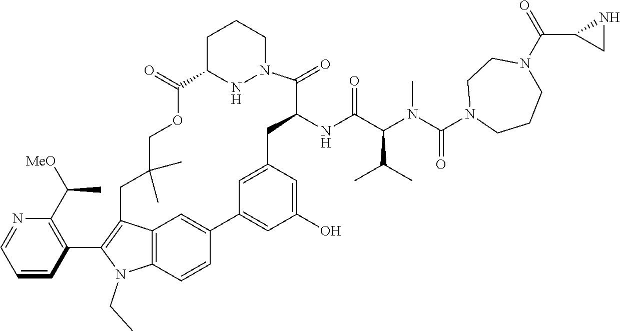 |
| C191 | 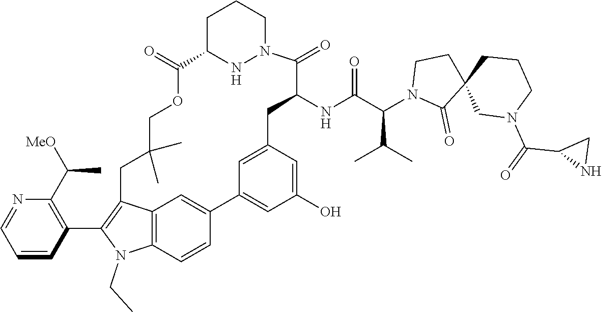 |
| C192 | 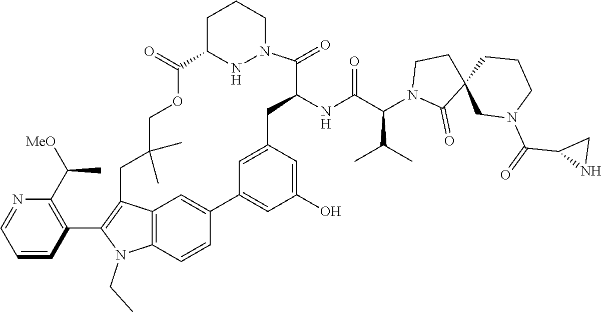 |
| C193 | 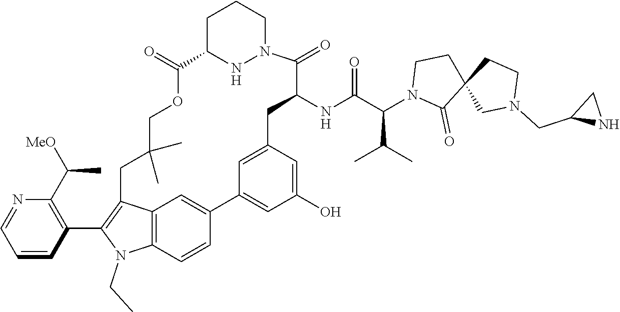 |
| C194 | 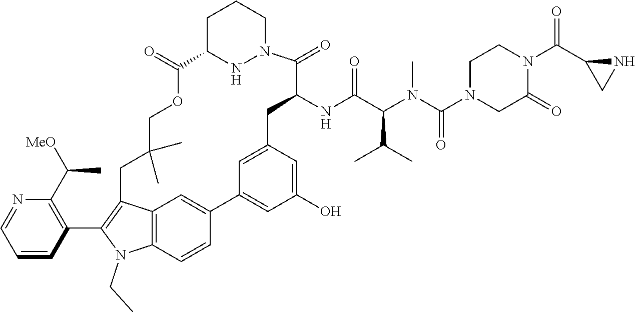 |
| C195 | 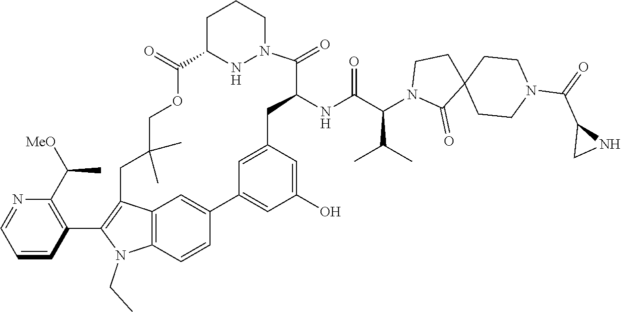 |
| C196 | 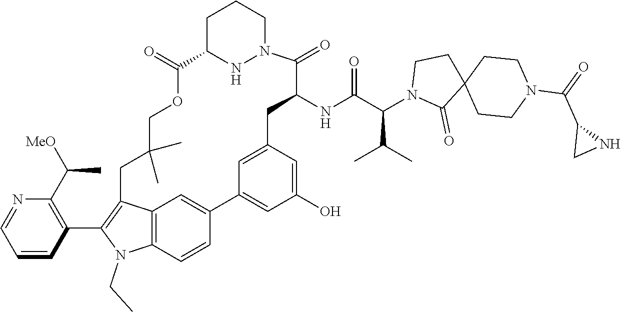 |
| C197 | 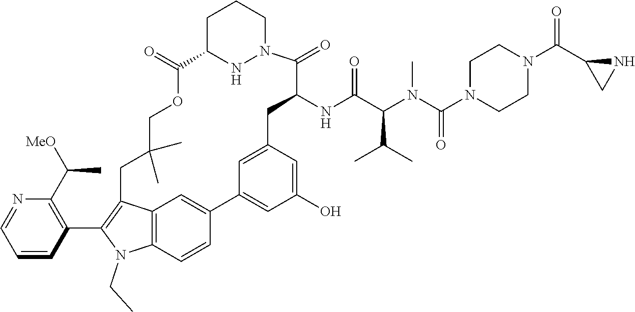 |
| C198 | 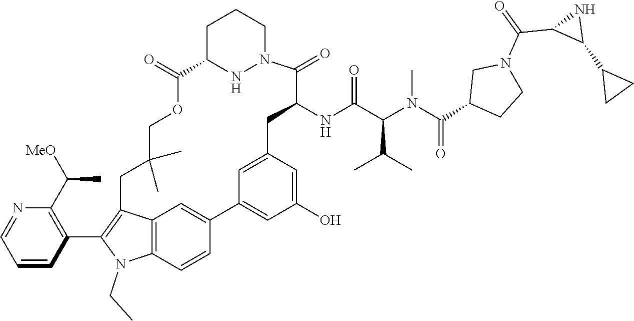 |
| C199 | 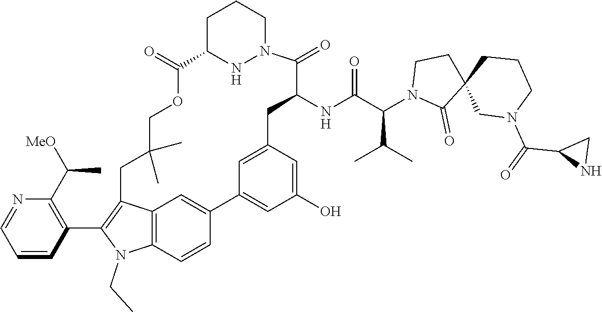 |
| C200 | 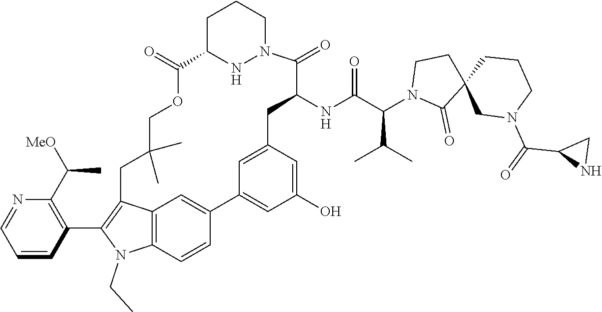 |
| C201 | 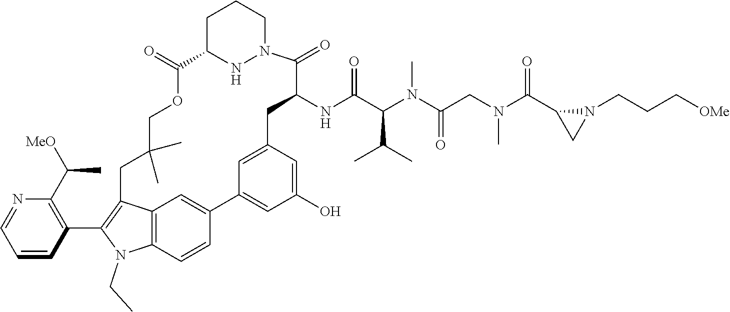 |
| C202 | 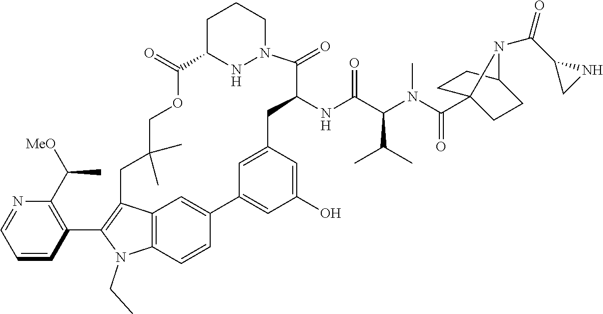 |
| C203 | 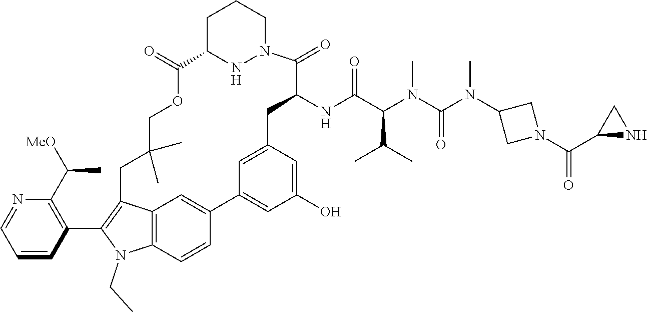 |
| C204 | 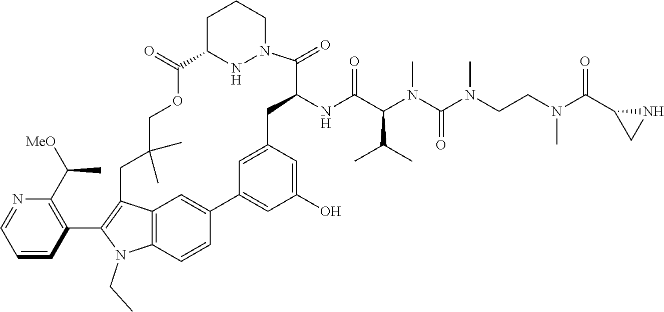 |
| C205 | 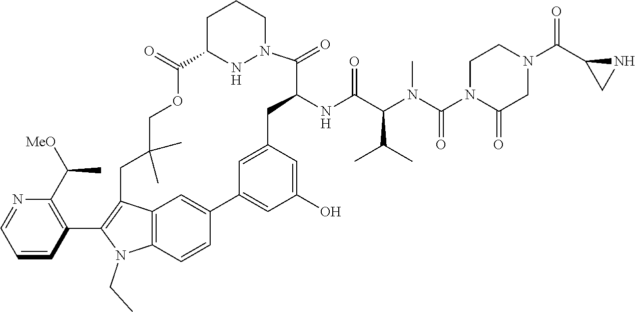 |
| C206 | 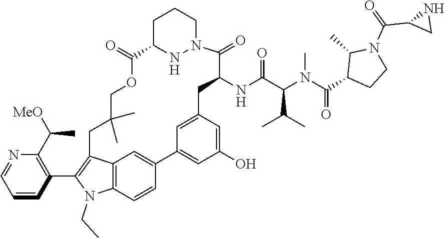 |
| C207 | 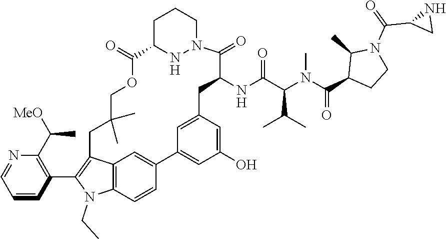 |
| C208 | 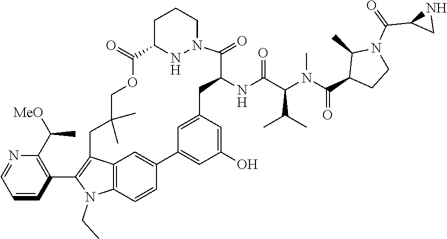 |
| C209 | 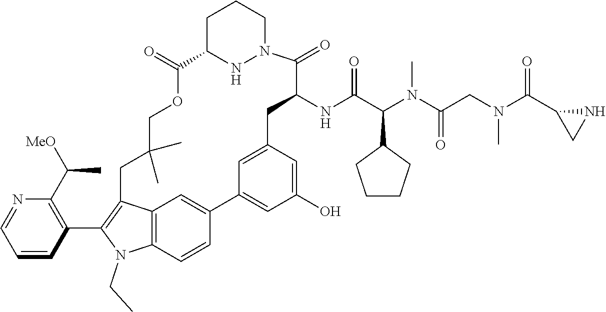 |
| C210 | 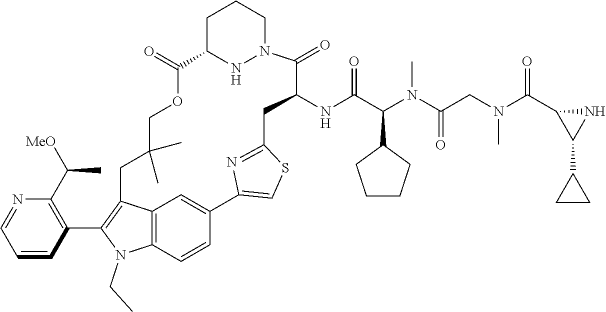 |
| C211 | 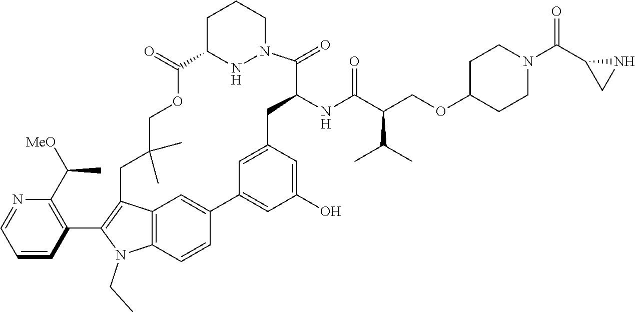 |
| C212 | 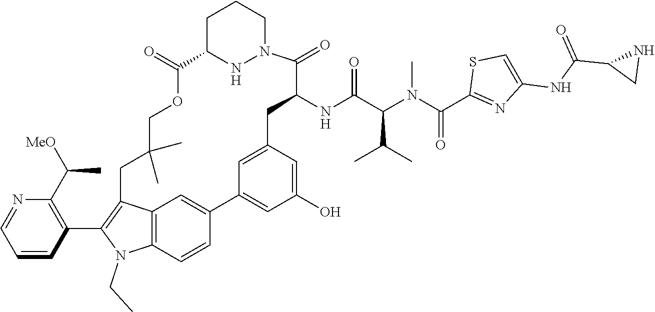 |
| C213 | 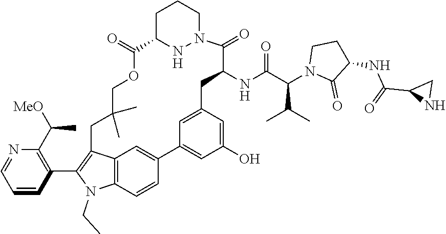 |
| C214 | 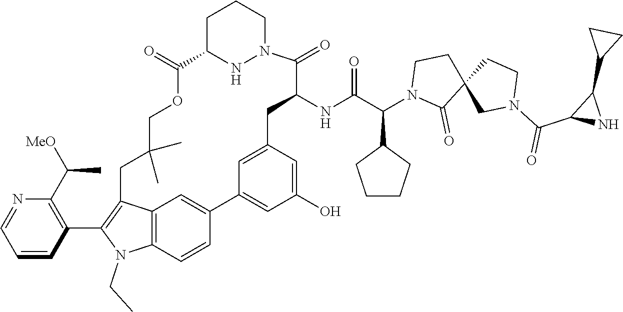 |
| C215 | 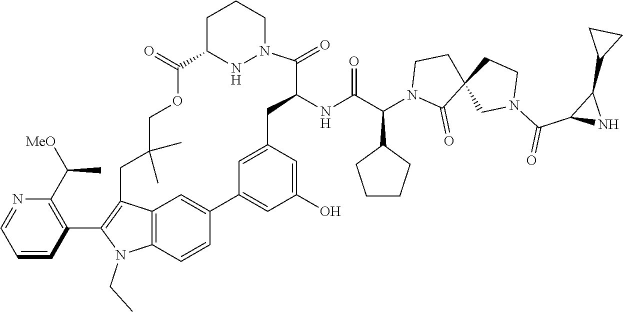 |
| C216 | 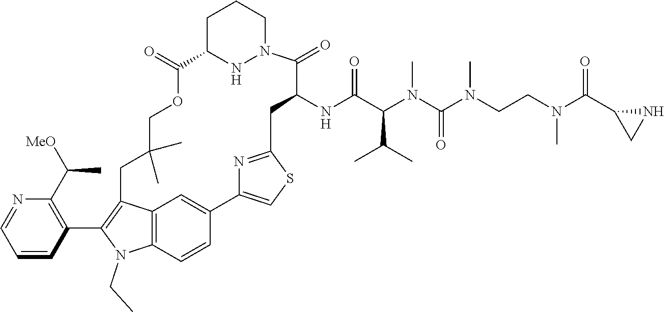 |
| C217 | 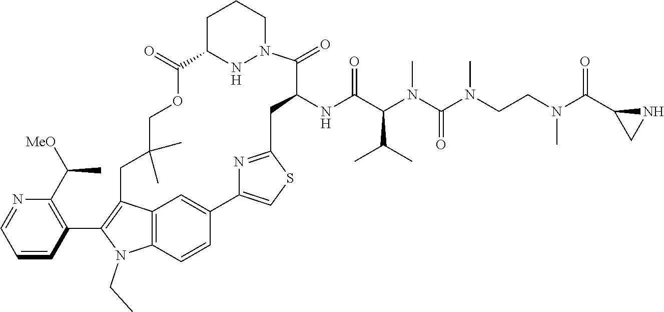 |
| C218 | 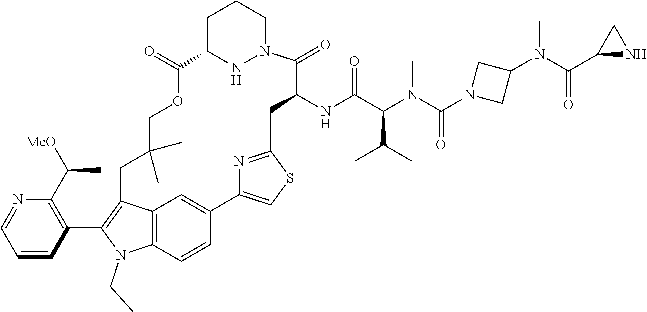 |
| C219 | 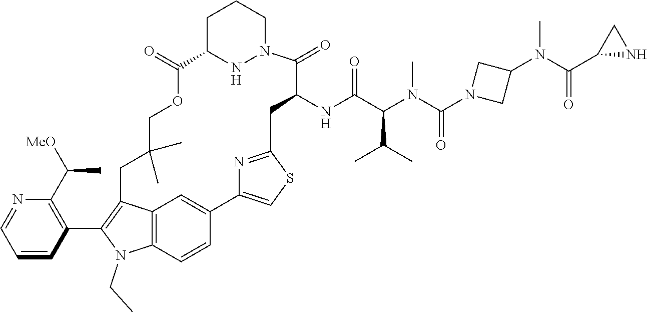 |
| C220 | 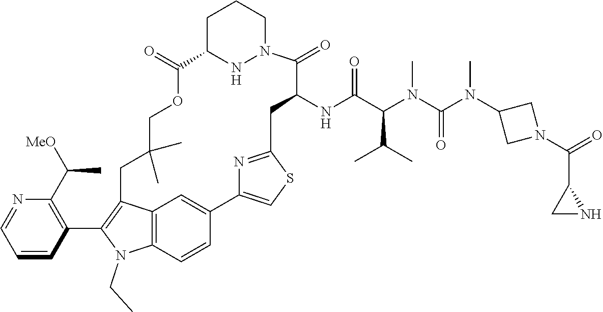 |
| C221 | 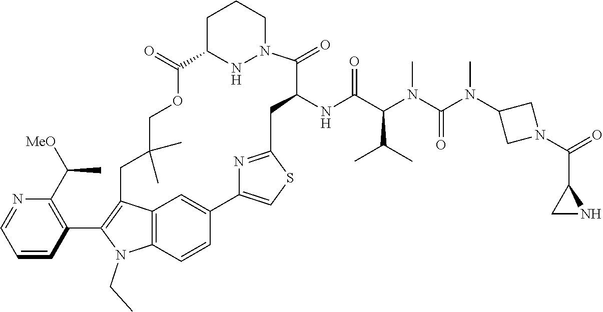 |
| C222 | 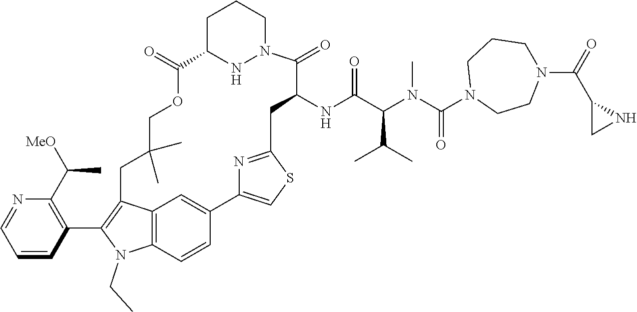 |
| C223 | 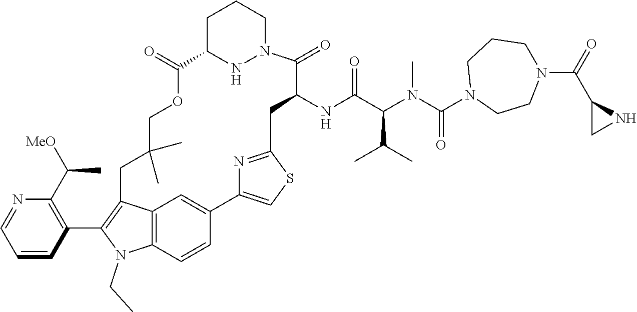 |
| C224 | 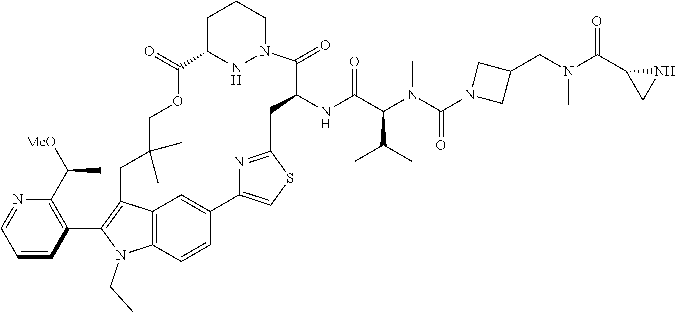 |
| C225 | 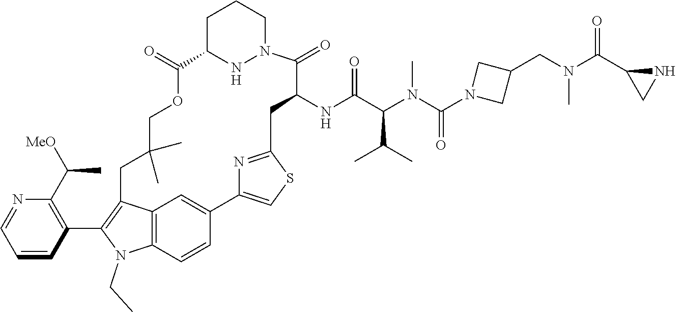 |
| C226 | 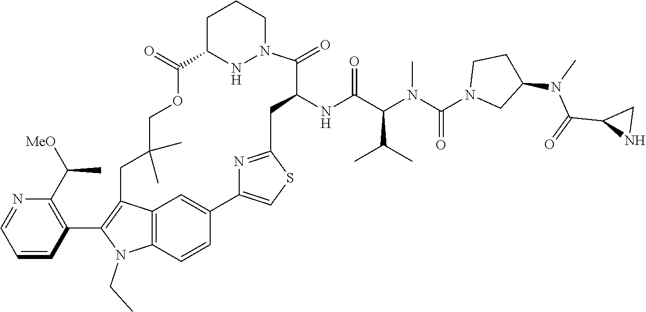 |
| C227 | 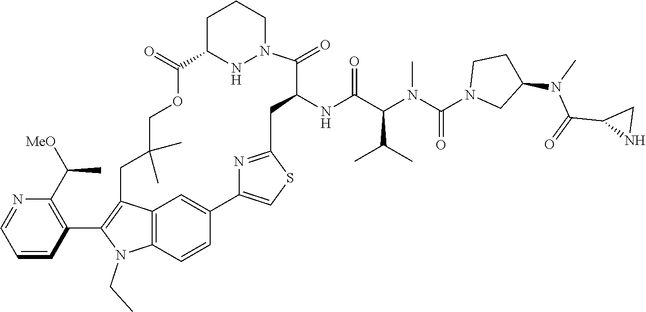 |
| C228 | 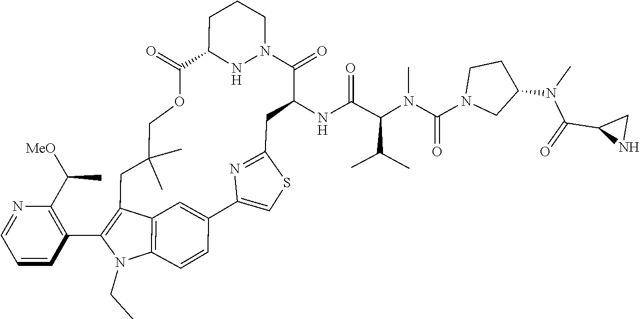 |
| C229 | 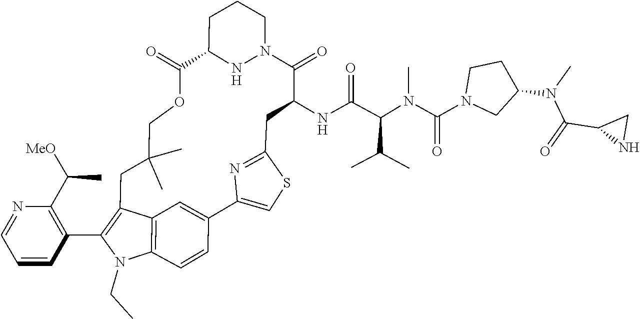 |
| C230 | 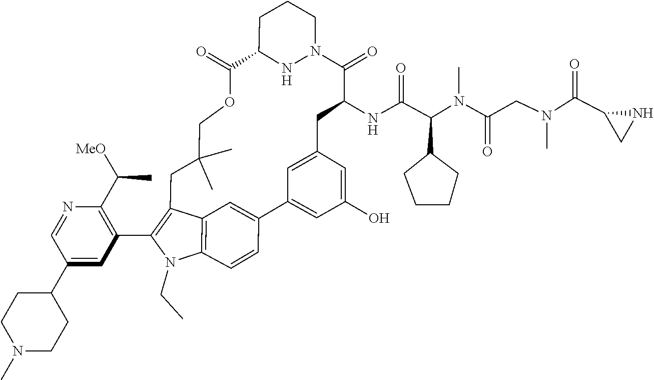 |
| C231 | 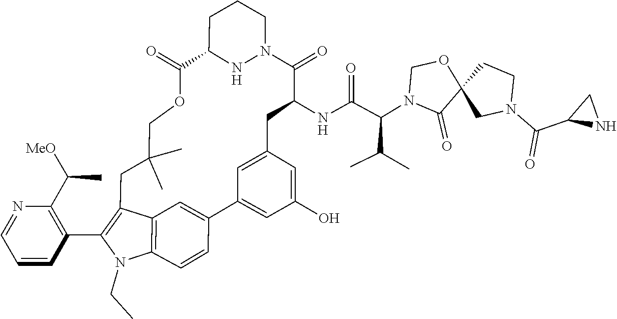 |
| C232 | 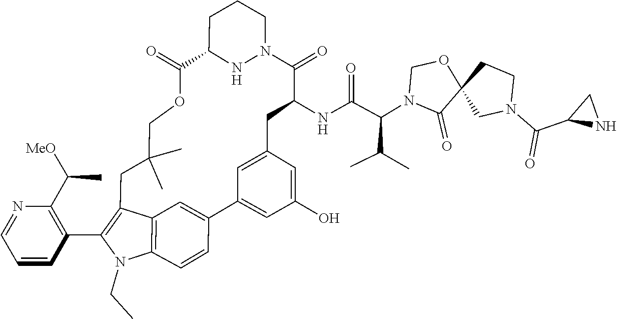 |
| C233 | 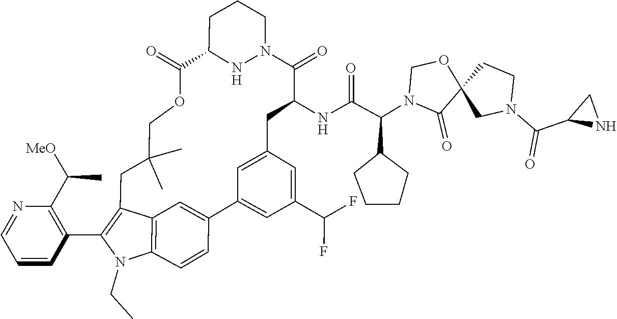 |
| C234 | 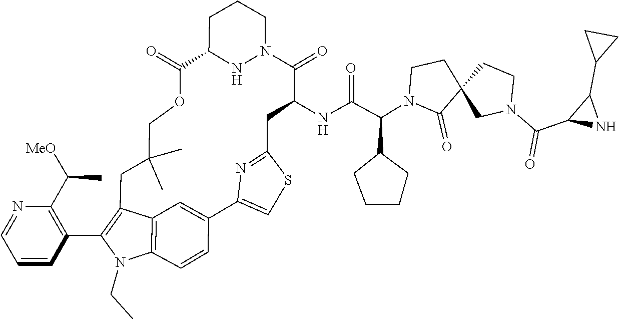 |
| C235 | 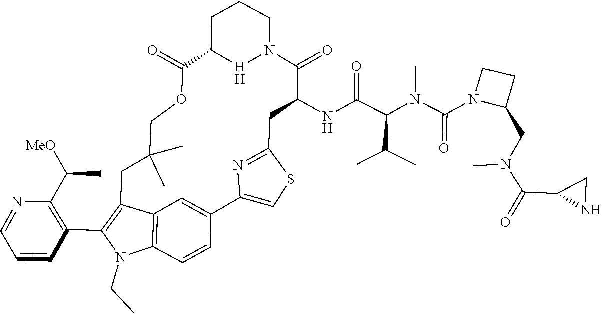 |
| C236 | 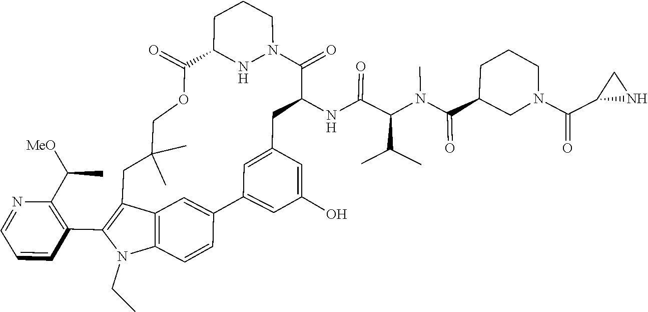 |
| C237 | 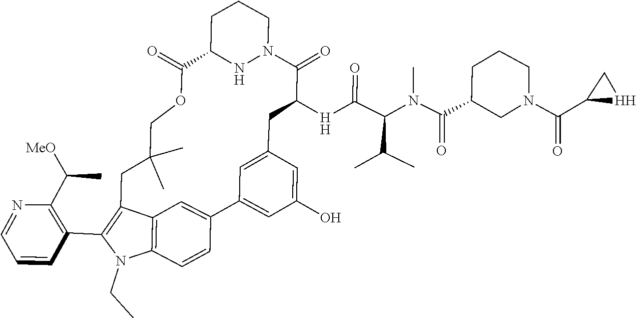 |
| C238 | 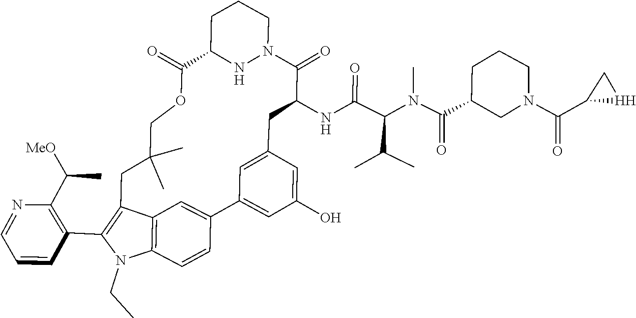 |
| C239 | 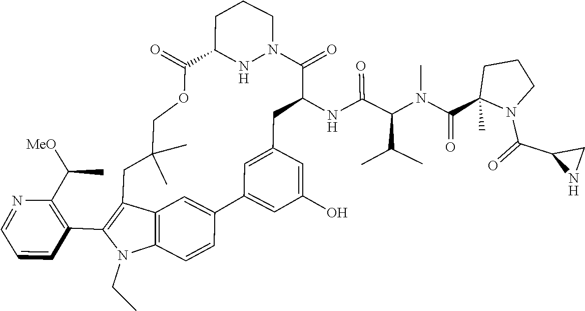 |
| C240 | 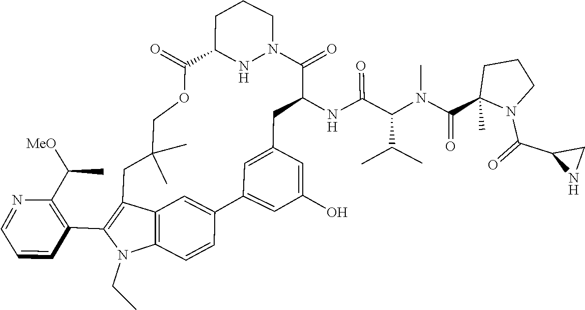 |
| C241 | 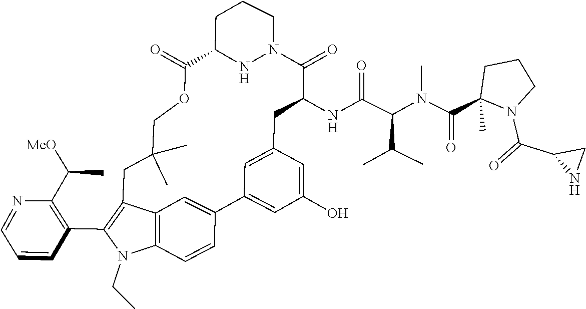 |
| C242 | 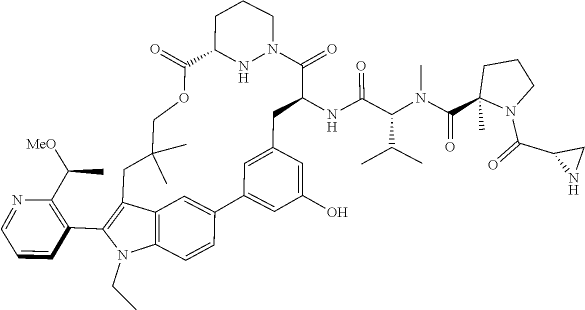 |
| C243 | 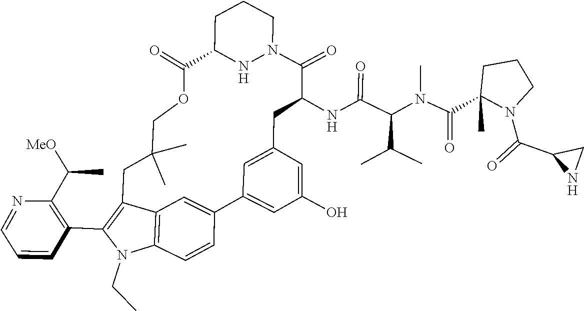 |
| C244 | 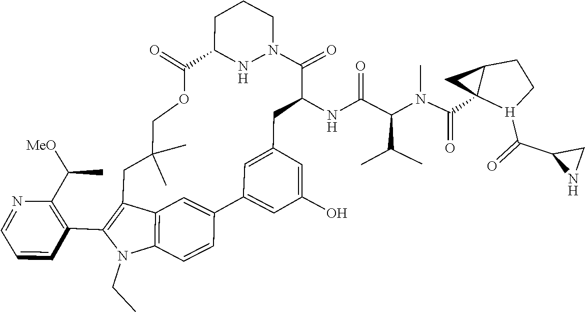 |
| C245 | 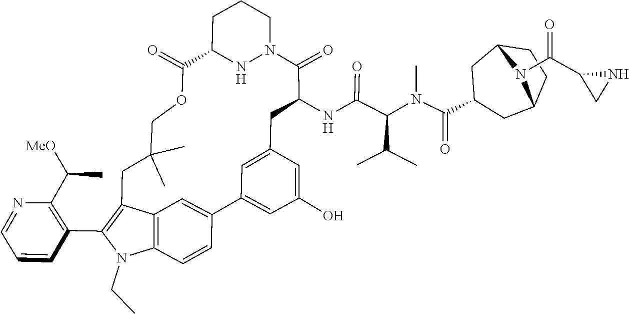 |
| C246 | 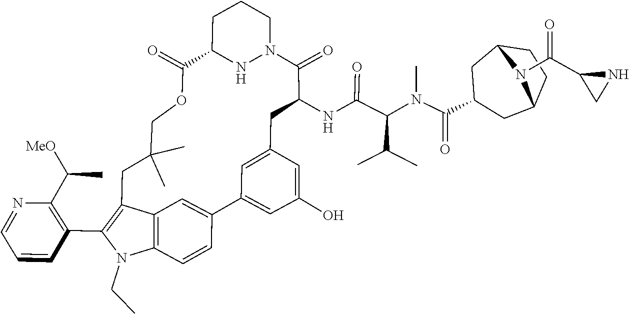 |
| C247 | 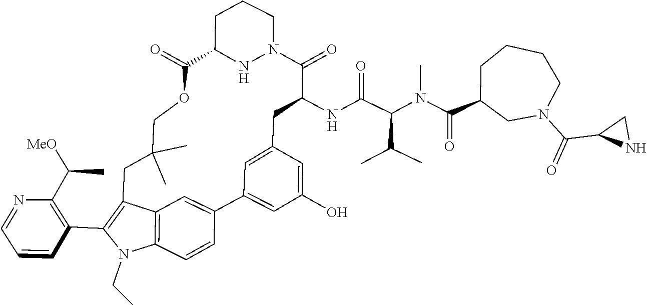 |
| C248 | 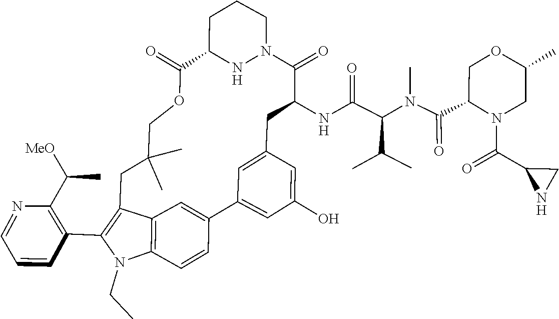 |
| C249 | 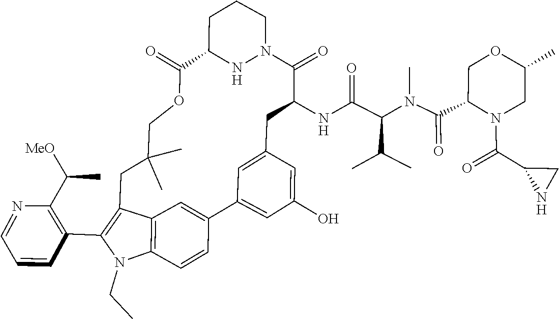 |
| C250 | 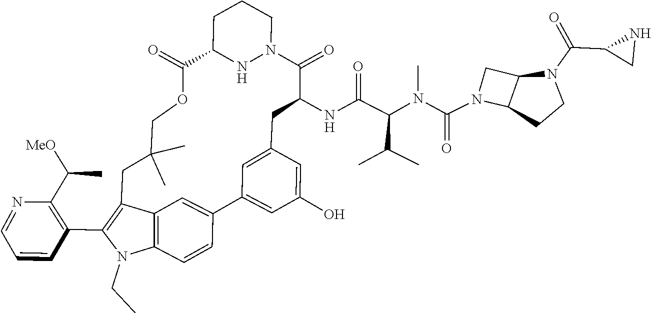 |
| C251 | 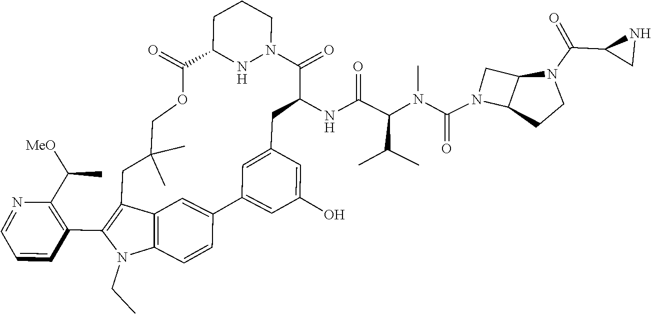 |
| C252 | 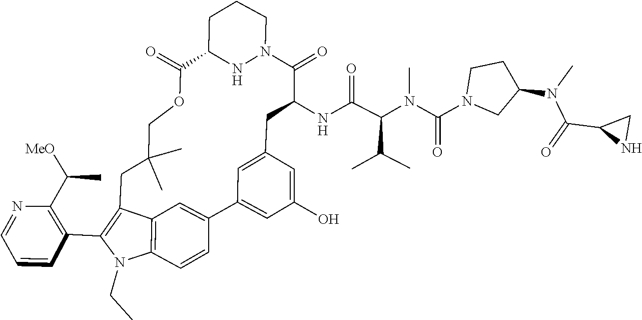 |
| C253 | 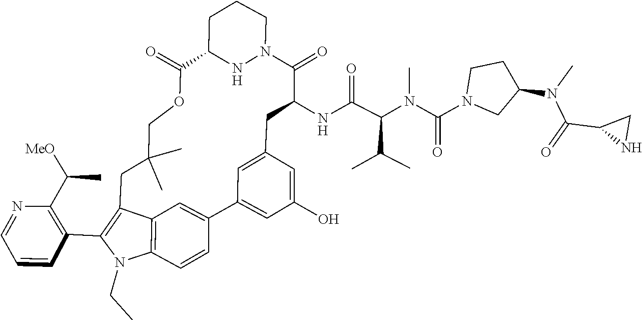 |
| C254 | 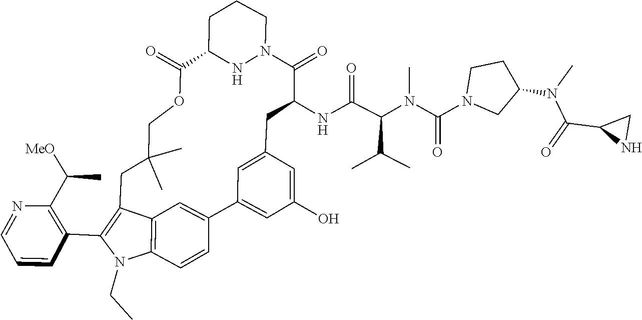 |
| C255 | 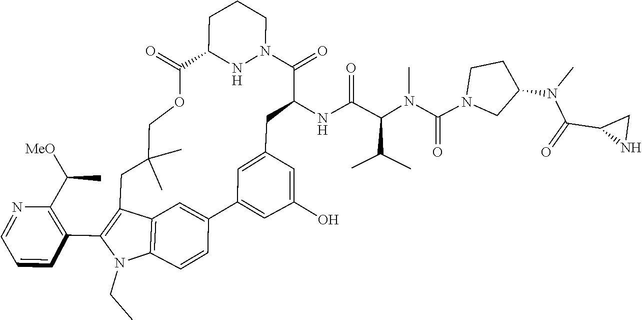 |
| C256 | 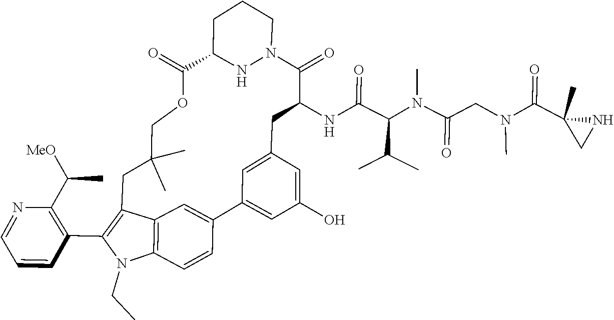 |
| C257 | 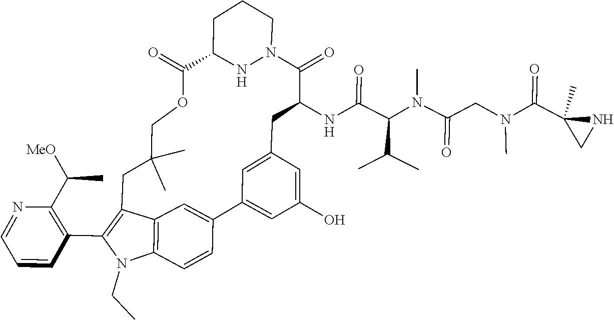 |
| C258* | 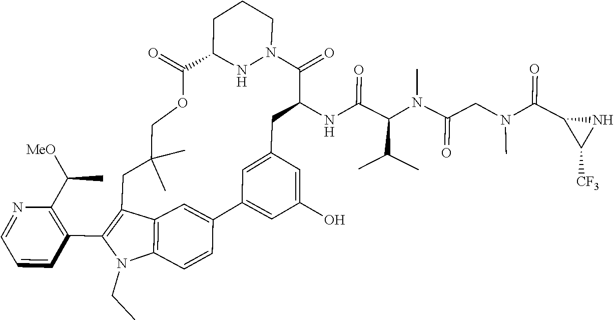 |
| C259* | 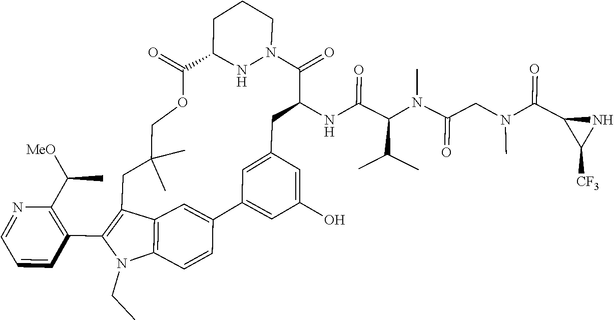 |
| C260* | 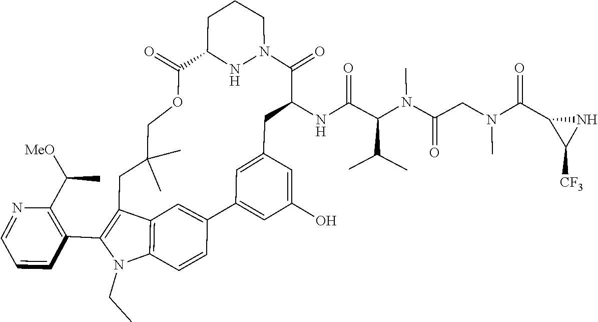 |
| C261* | 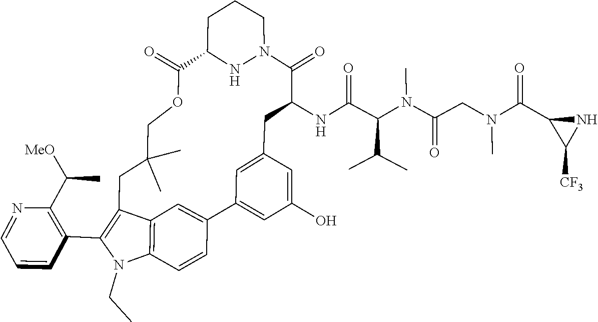 |
| C262 | 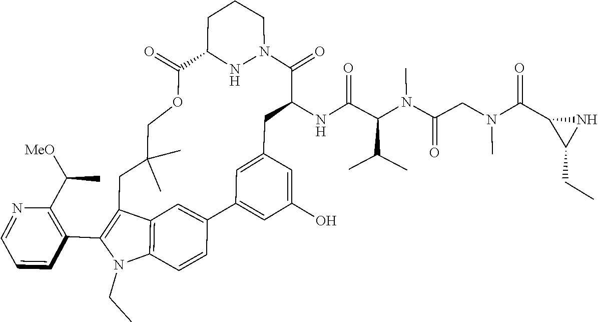 |
| C263 | 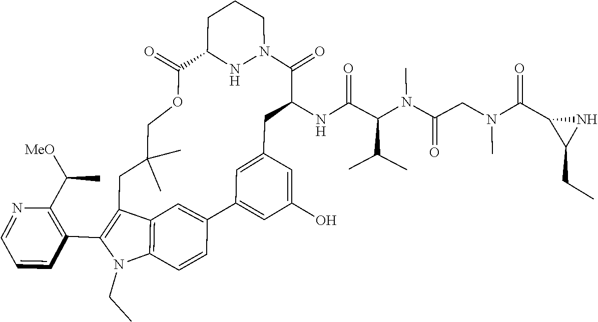 |
| C264 | 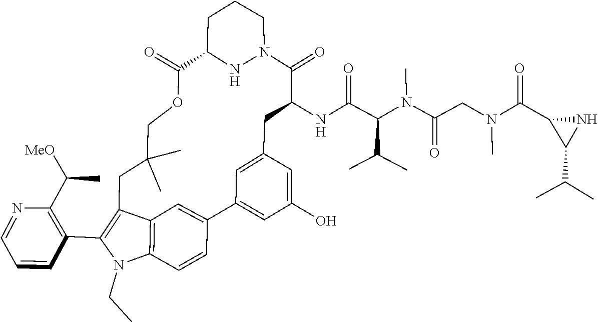 |
| C265 | 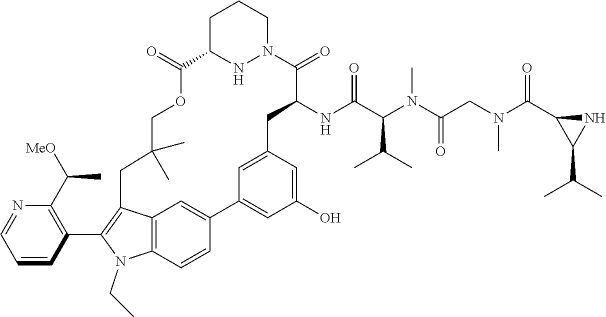 |
| C266 | 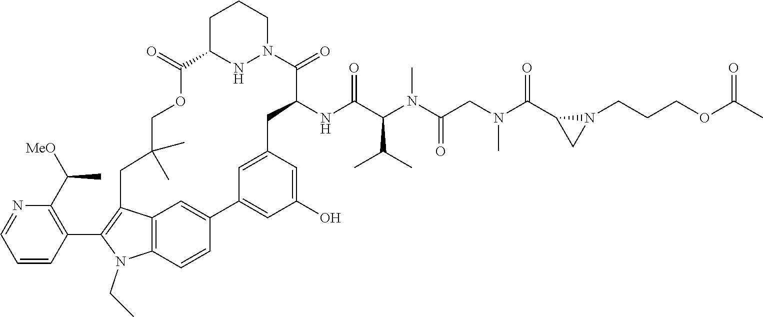 |
| C267 | 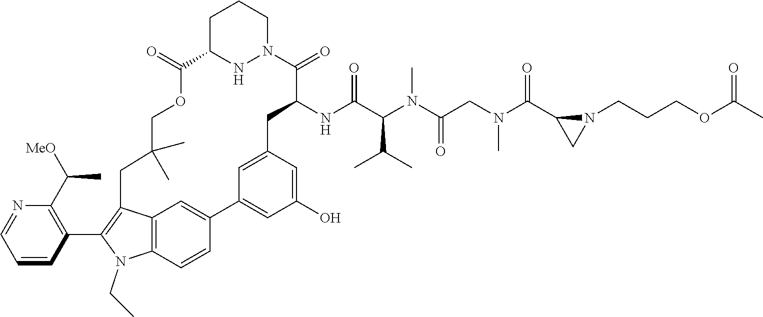 |
| C268 | 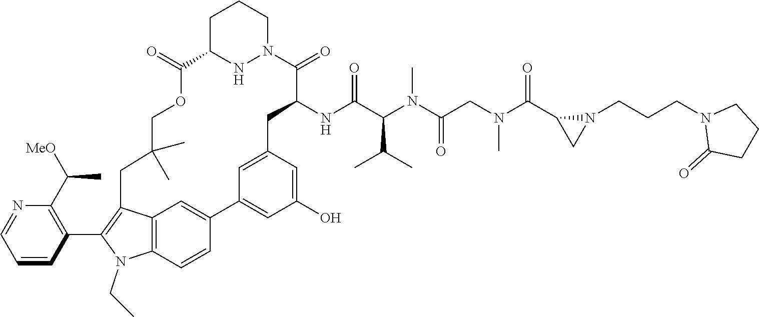 |
| C269 | 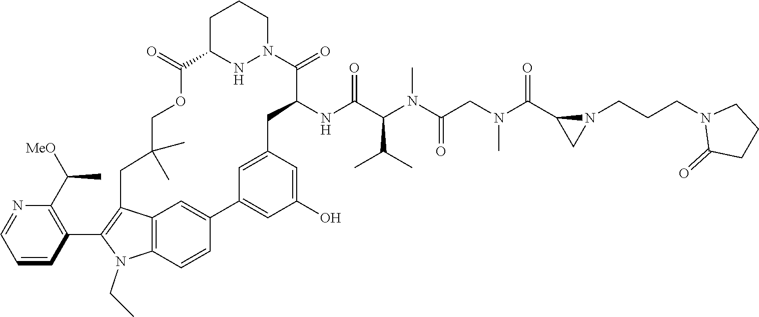 |
| C270 | 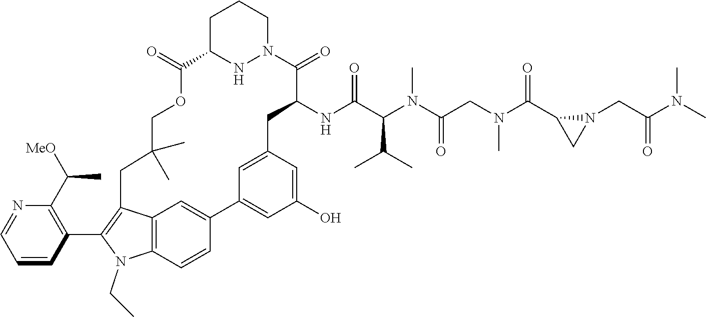 |
| C271 | 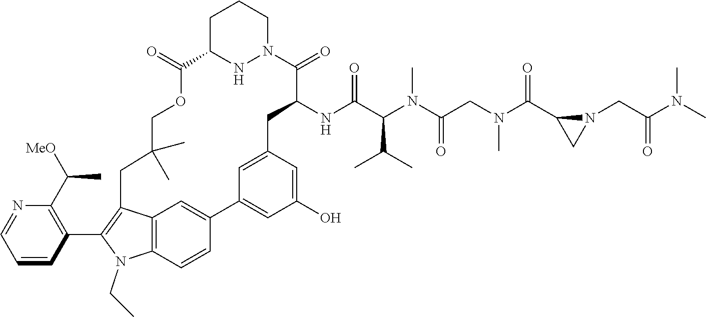 |
| C272 | 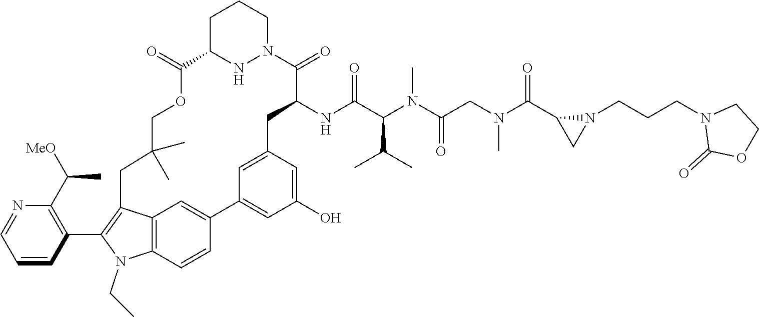 |
| C273 | 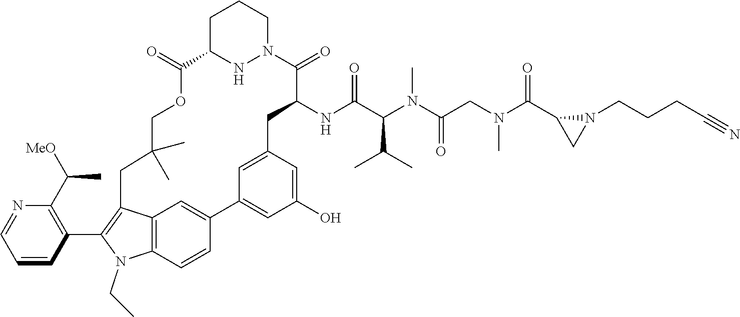 |
| C274 | 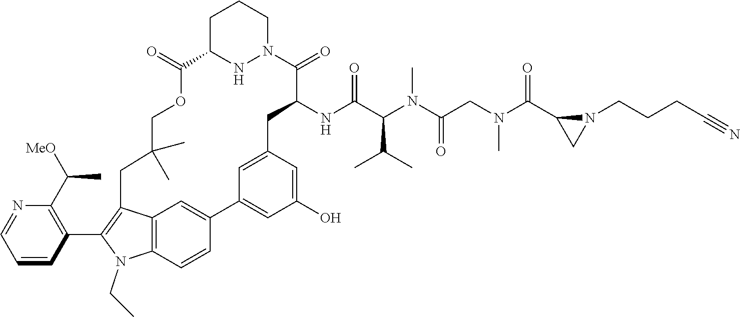 |
| C275 | 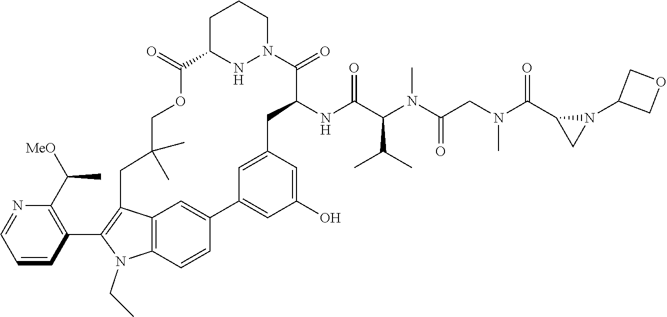 |
| C276 | 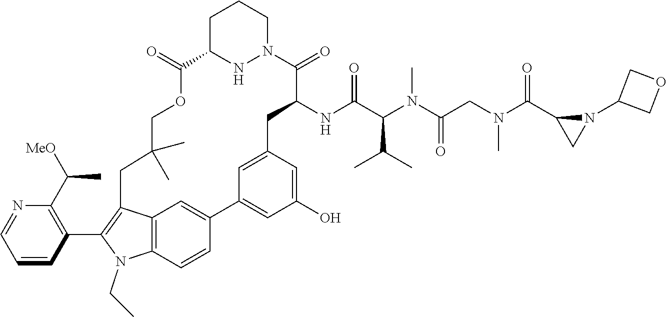 |
| C277 | 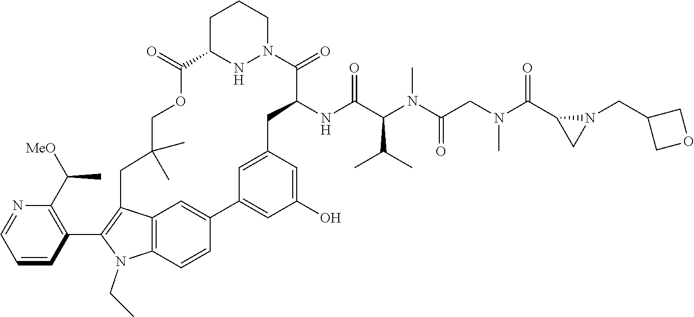 |
| C278 | 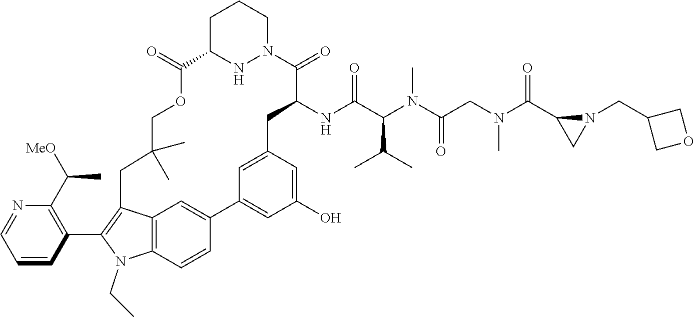 |
| C279 | 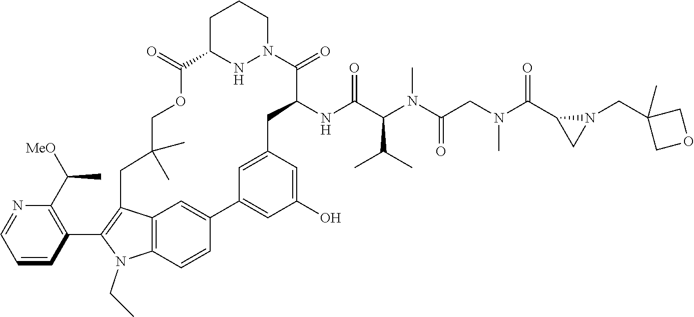 |
| C280 | 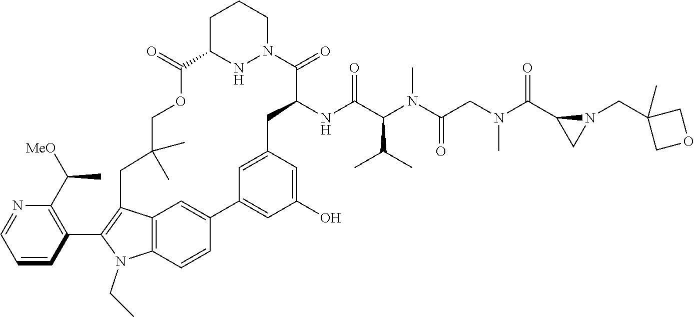 |
| C281* | 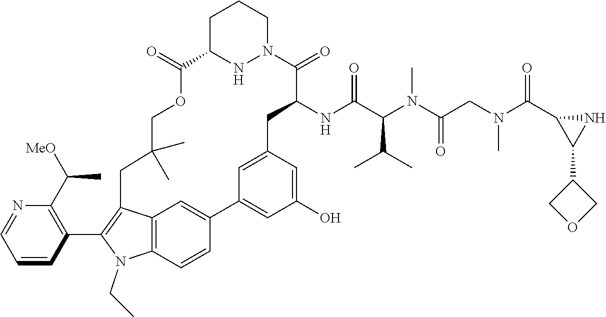 |
| C282* | 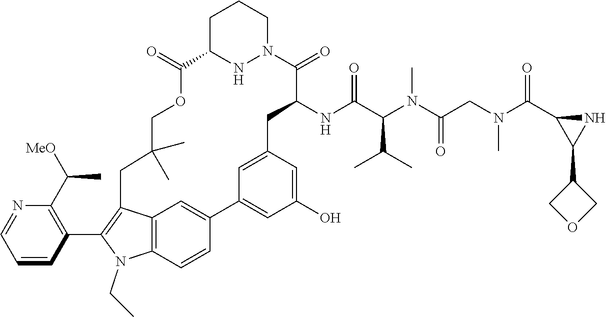 |
| C283* | 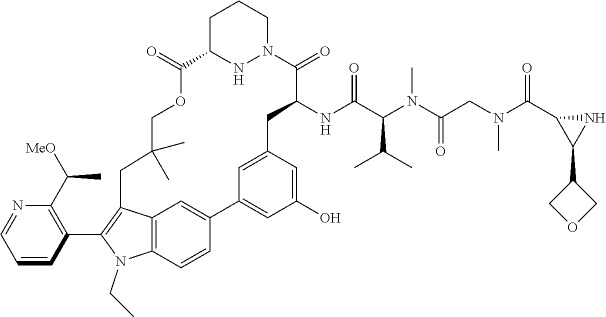 |
| C284* | 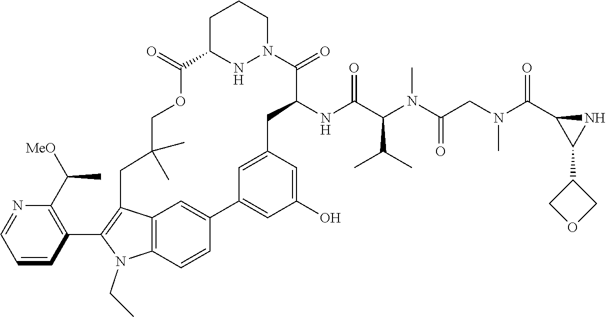 |
| C285 | 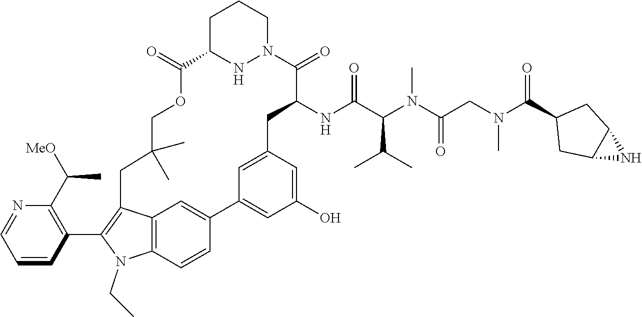 |
| C286 | 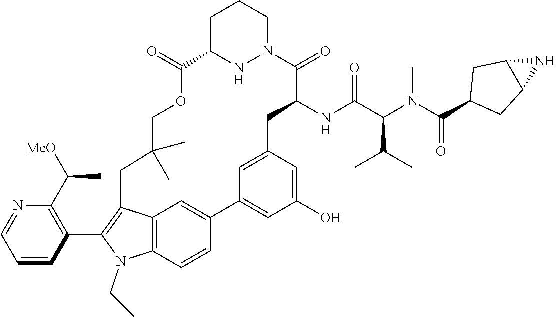 |
| C287 | 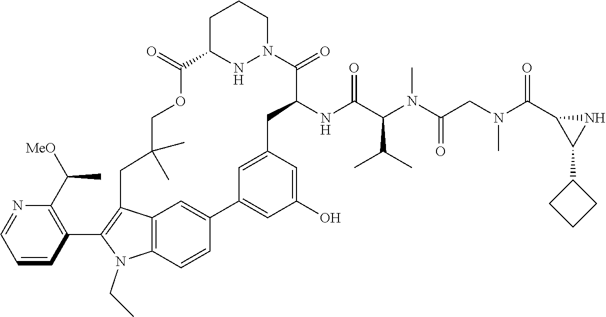 |
| C288 | 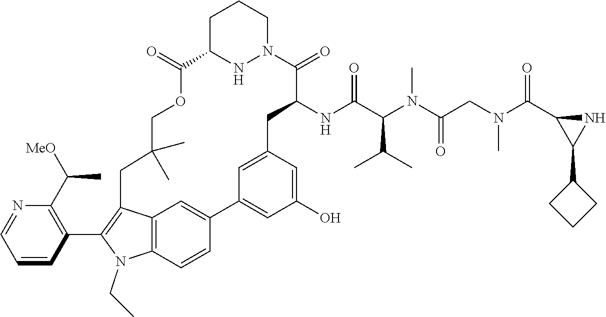 |
| C289 | 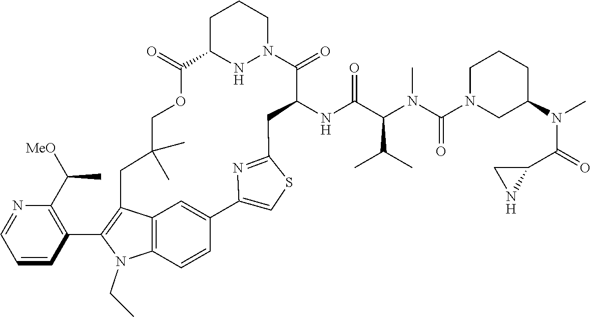 |
| C290 | 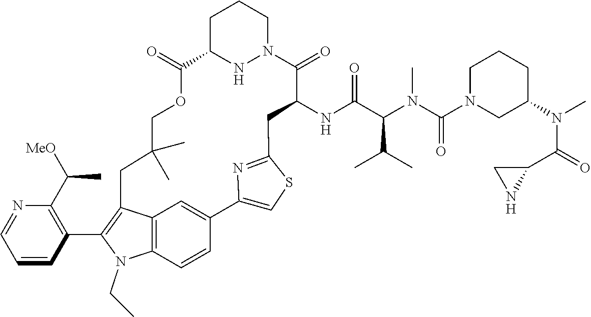 |
| C291 | 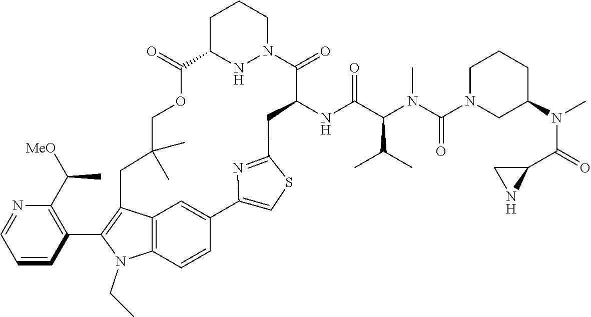 |
| C292 | 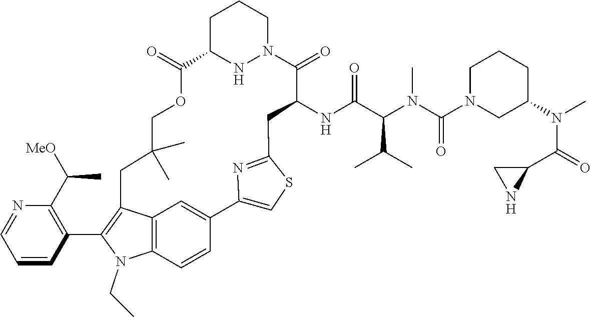 |
| C293 | 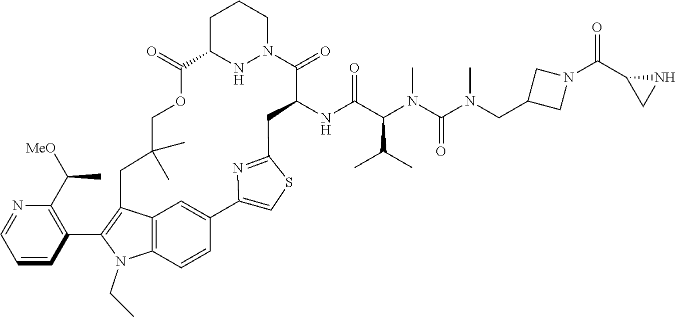 |
| C294 | 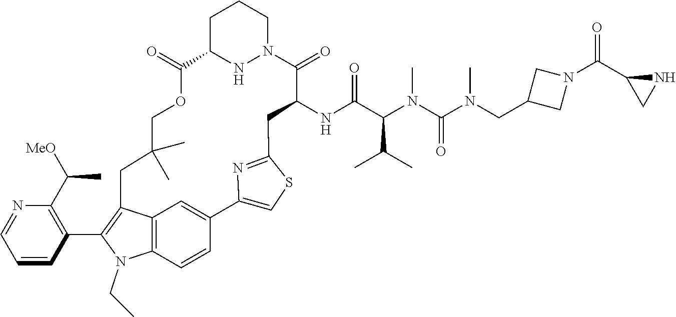 |
| C295 | 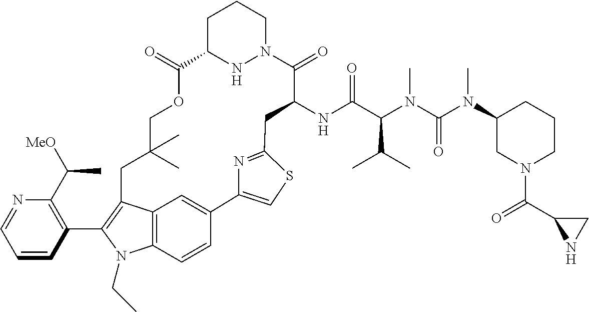 |
| C296 | 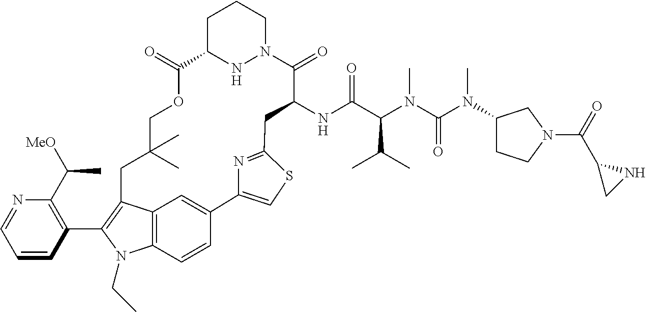 |
| C297 | 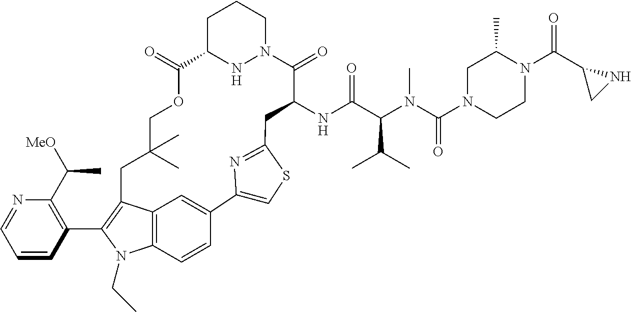 |
| C298 | 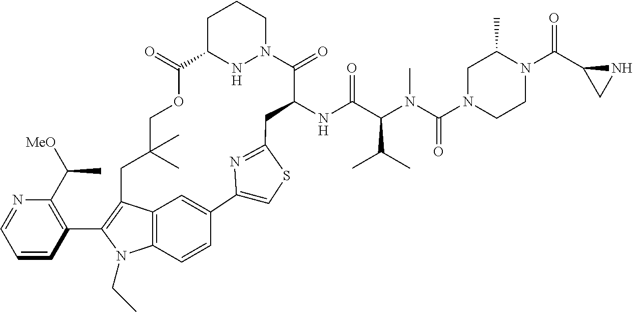 |
| C299 | 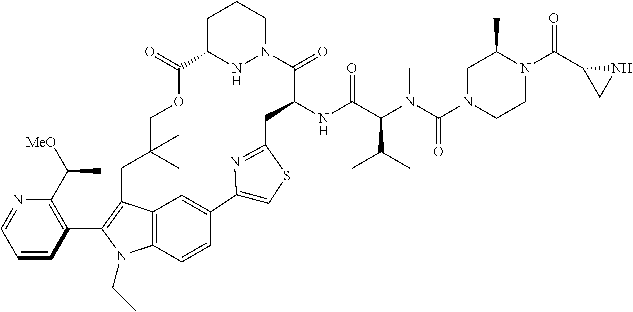 |
| C300 | 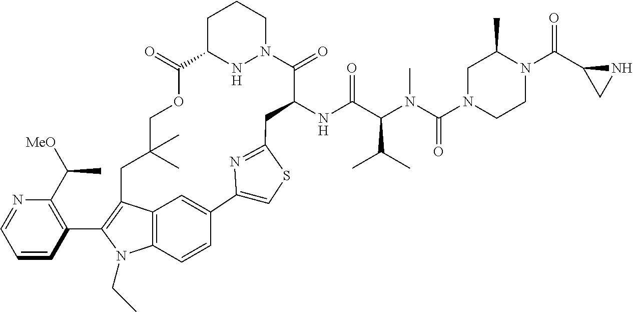 |
| C301 | 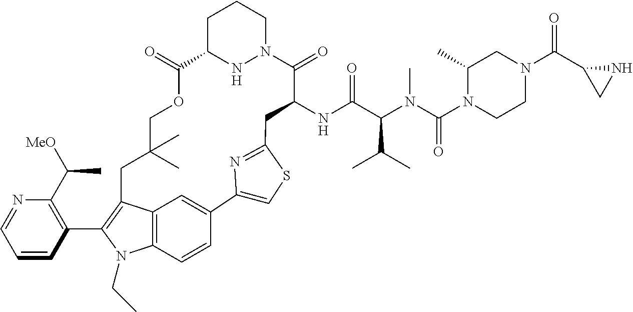 |
| C302 | 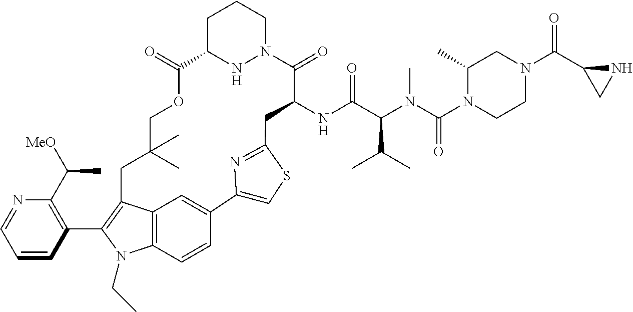 |
| C303 | 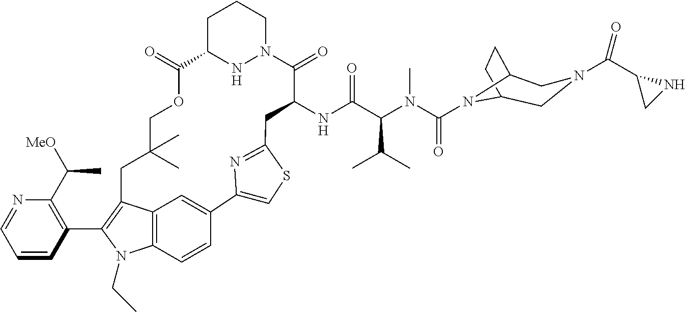 |
| C304 | 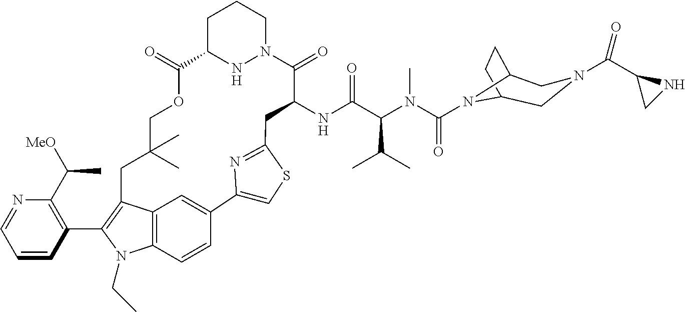 |
| C305 | 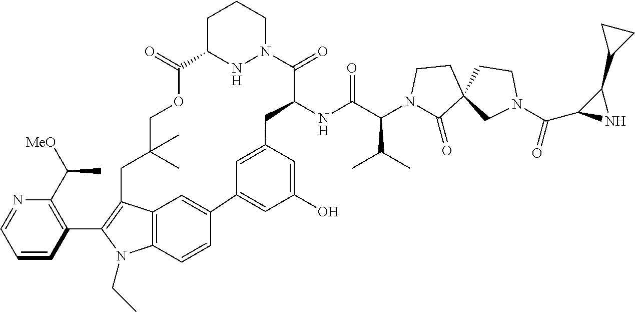 |
| C306 | 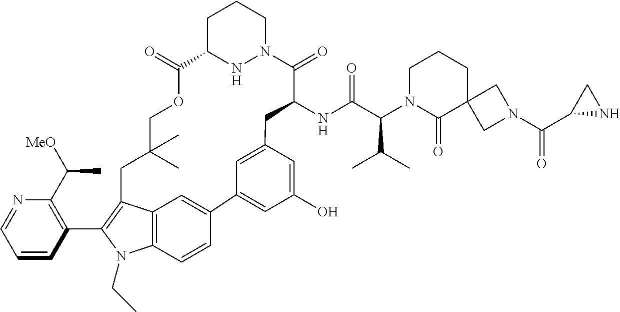 |
| C307 | 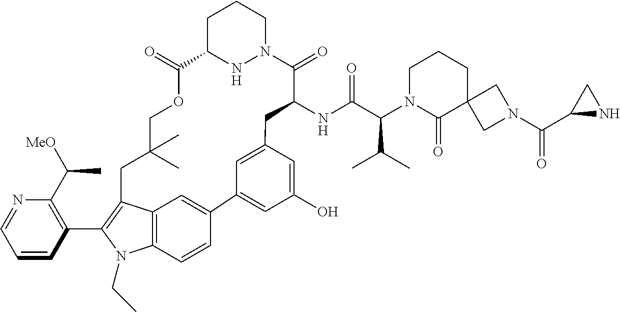 |
| C308* | 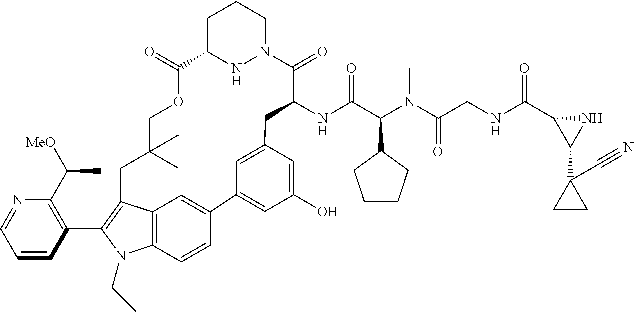 |
| C309 | 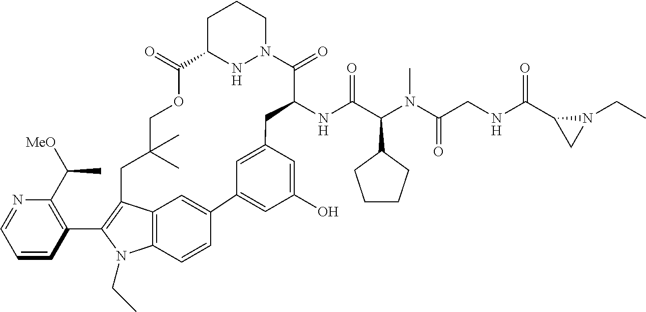 |
| C310 | 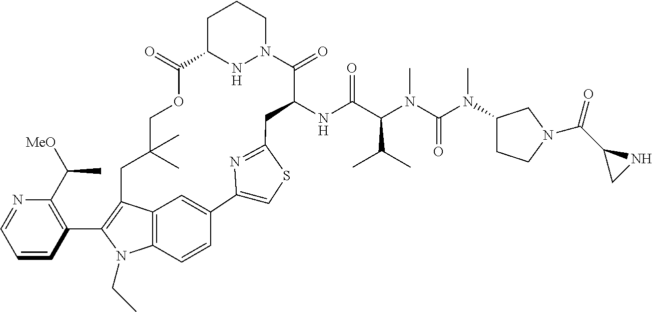 |
| C311 | 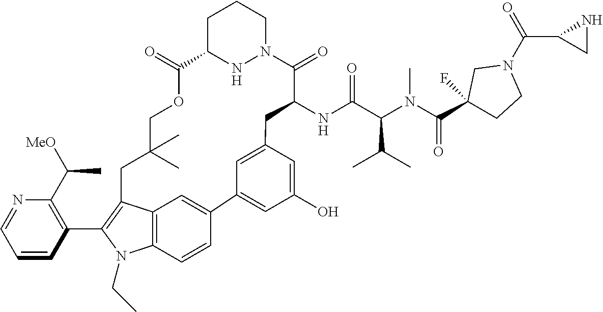 |
| C312 | 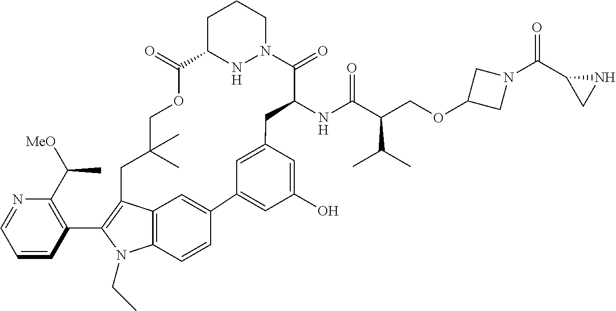 |
| C313 | 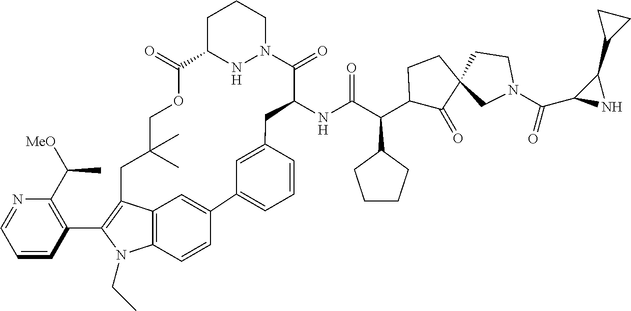 |
| C314* | 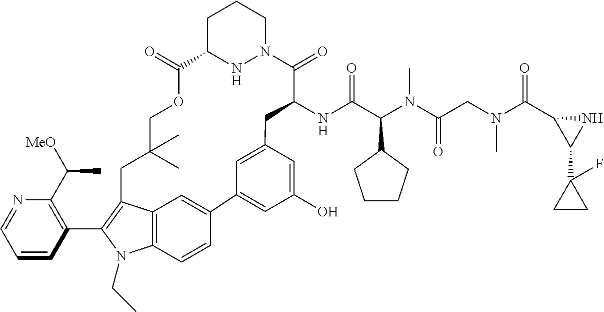 |
| C315* | 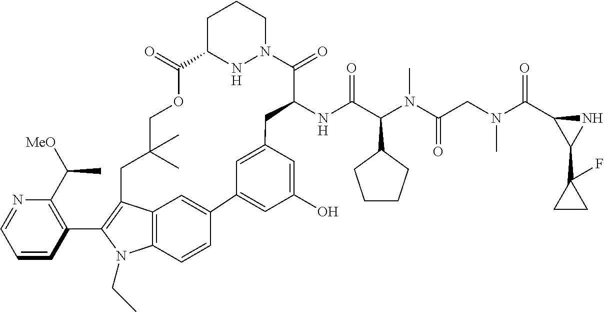 |
| C316* | 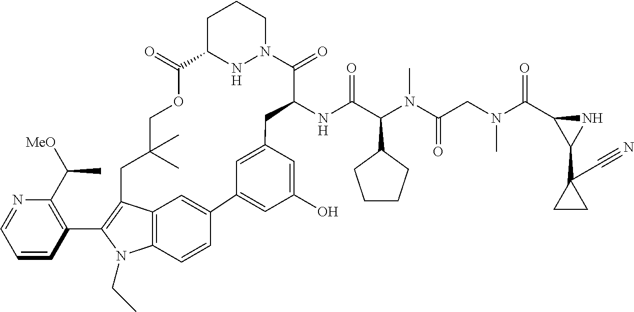 |
| C317 | 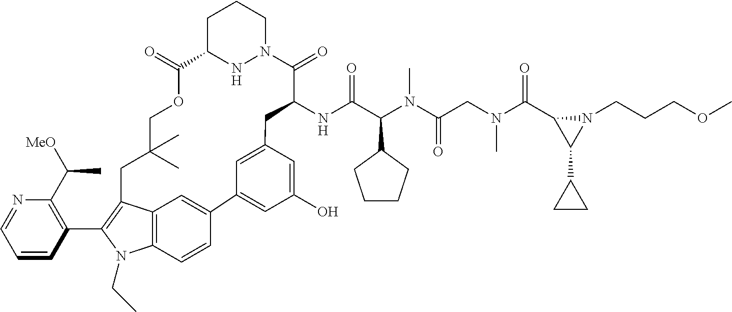 |
| C318 | 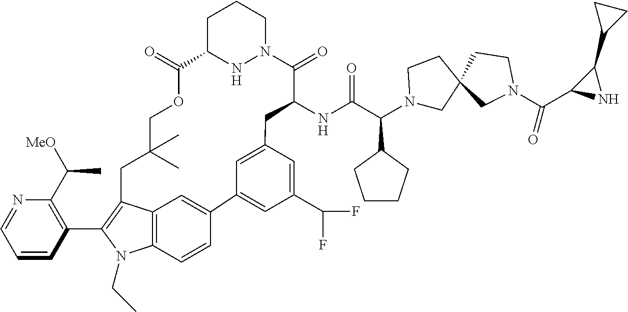 |
| C319 | 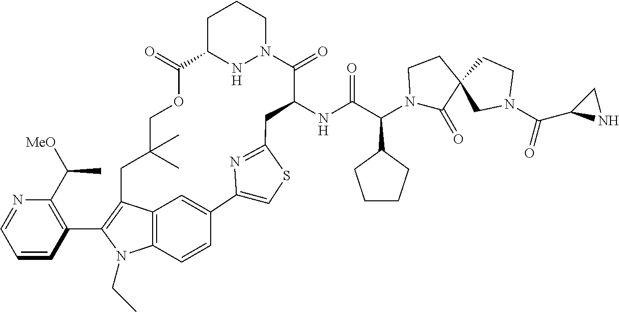 |
| C320 | 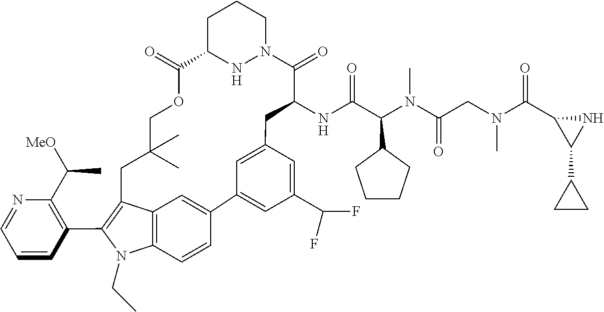 |
| C321 | 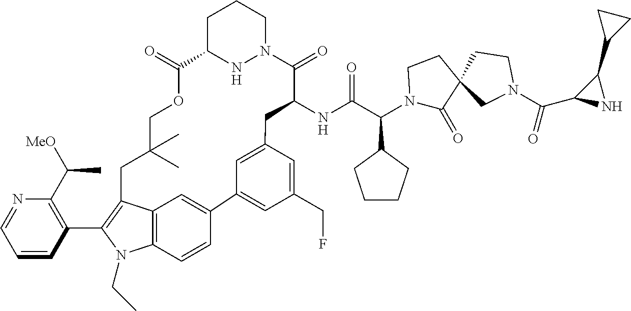 |
| C322* | 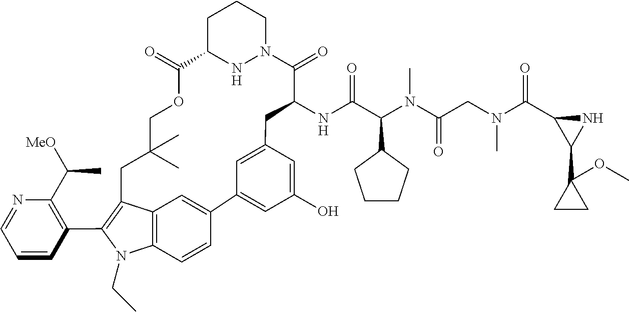 |
| C323* | 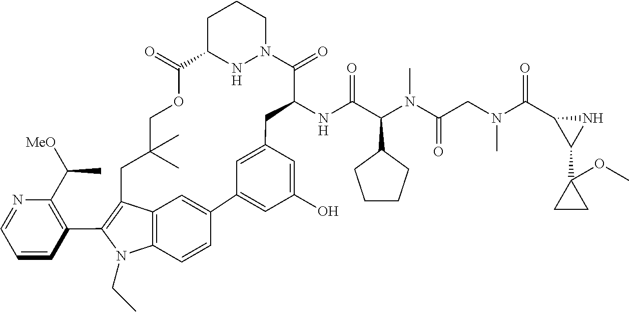 |
| C324 | 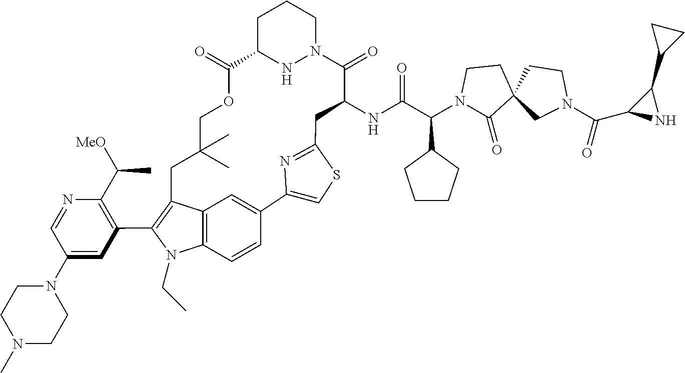 |
| C325 | 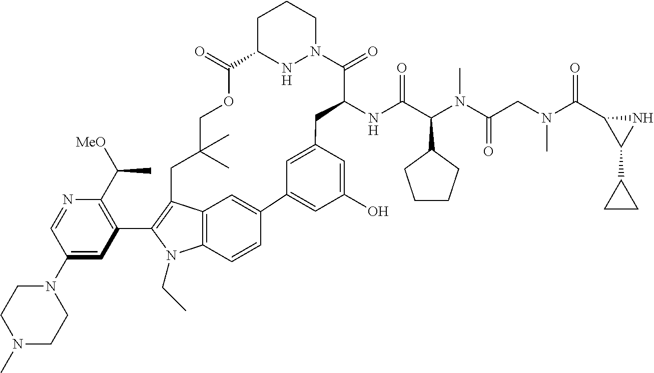 |
| C326 | 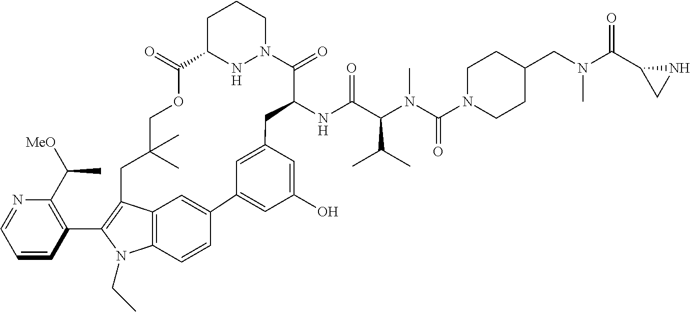 |
| C327 | 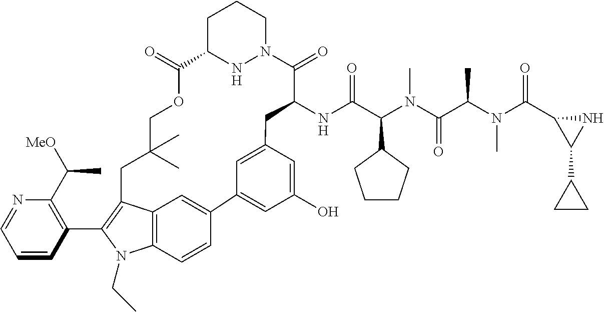 |
| C328 | 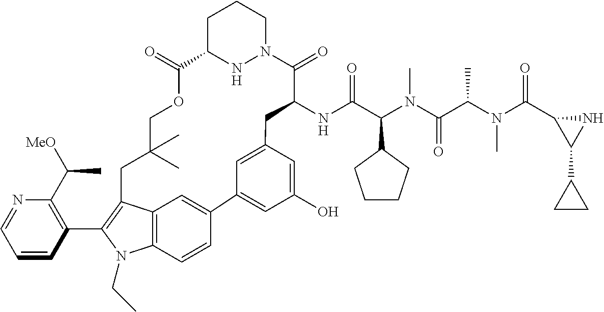 |
| C329 | 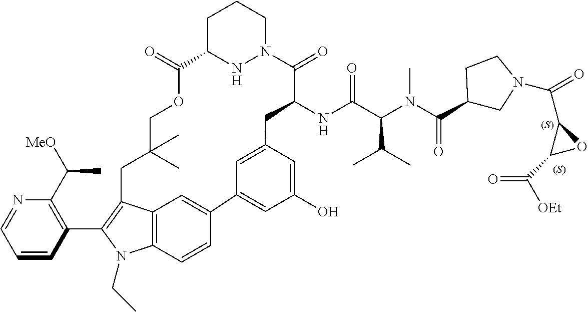 |
| C330 | 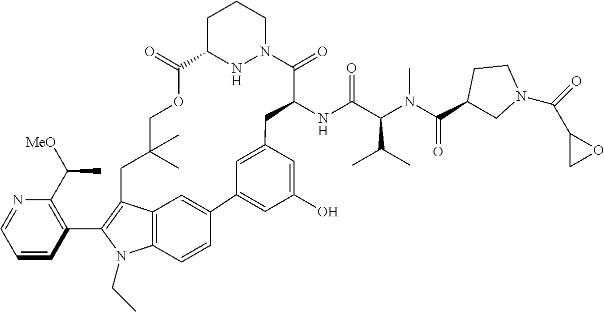 |
| C331 | 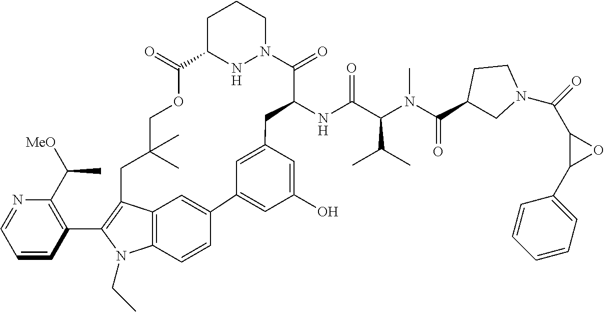 |
| C332 | 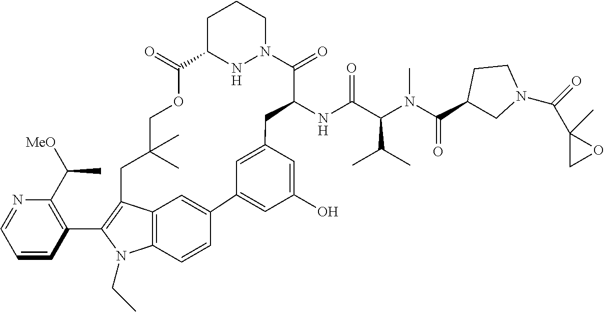 |

*Stereochemistry of the aziridine carbon is assumed.
Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated.

In some embodiments, a compound of Table C2 is provided, or a pharmaceutically acceptable salt thereof. In some embodiments, the RAS(ON) inhibitor is selected from Table C₂, or a pharmaceutically acceptable salt or atropisomer thereof.

TABLE C2
Certain Compounds of the Present Invention

| Ex# | Structure |
|---|---|
| CB5 | 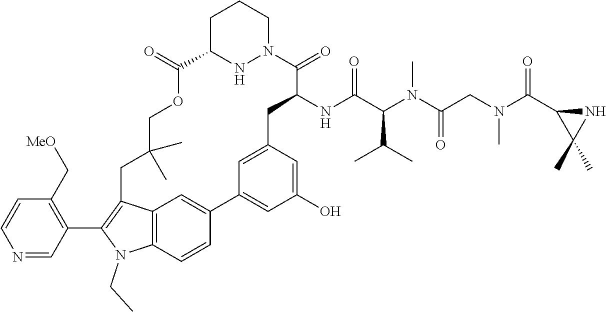 |
| CB6 | 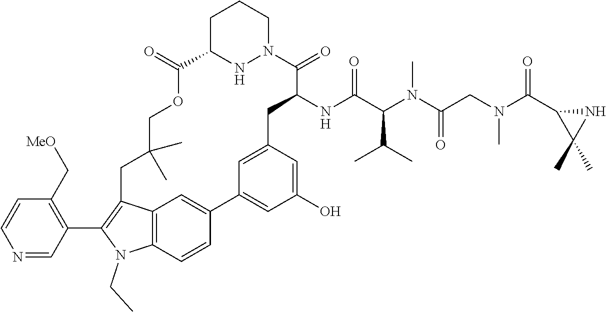 |
| CB8 | 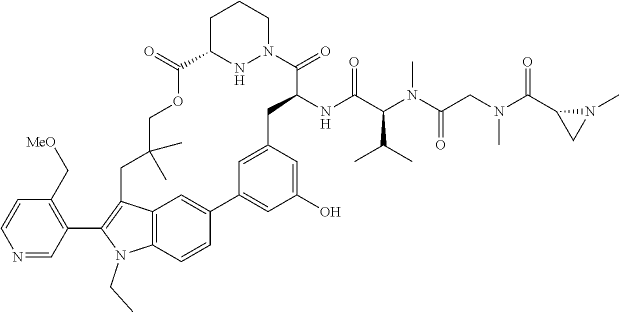 |
| CB9 | 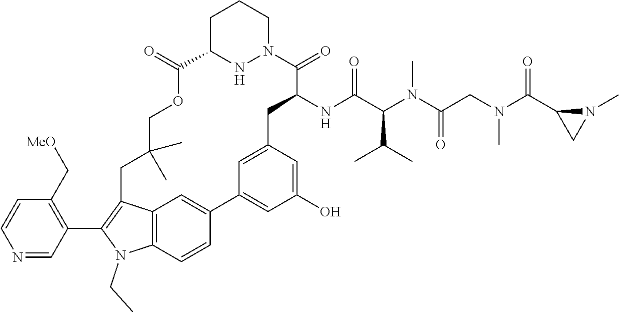 |
| CB16 | 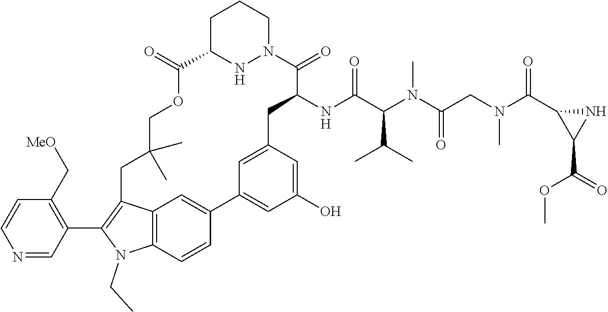 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C(=O)OC)C(=O)N1CCC[C@H](N1)C(=O)OCC(C)(C)C2] |
| CB19 | 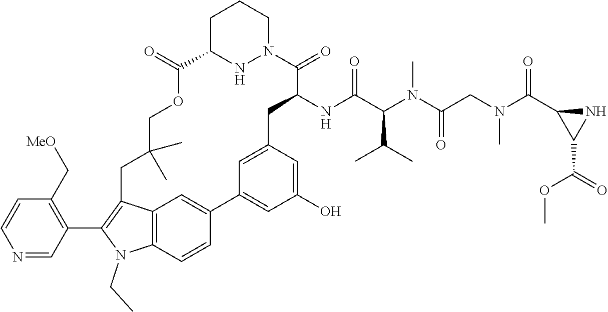 |
| CB29 | 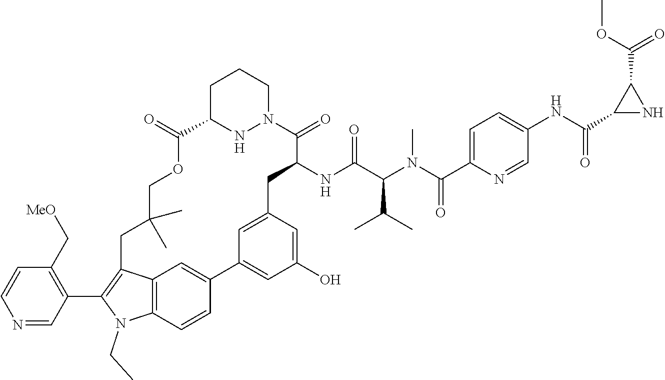 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3N[C@H]3C(=O)OC)cn1)C(=O)N1CCC[C@H](N1)C(=O)OCC(C)(C)C2] |
| CB30 | 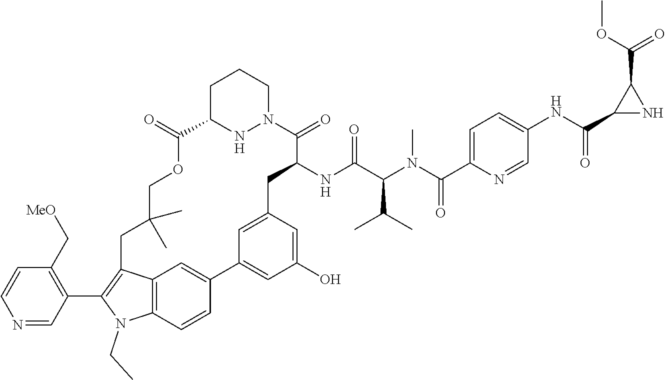 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3N[C@@H]3C(=O)OC)cn1)C(=O)N1CCC[C@H](N1)C(=O)OCC(C)(C)C2] |
| CB31 | 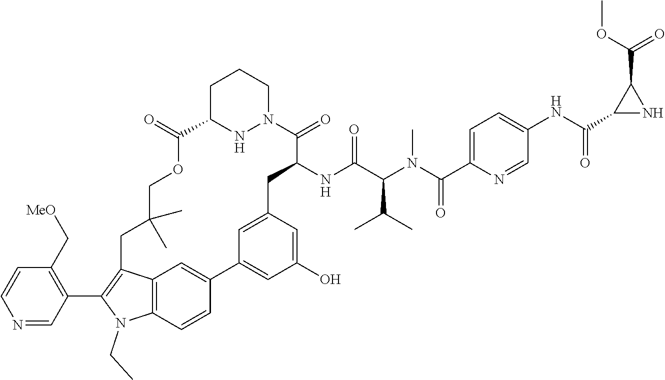 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@H]3N[C@@H]3C(=O)OC)cn1)C(=O)N1CCC[C@H](N1)C(=O)OCC(C)(C)C2] |
| CB32 | 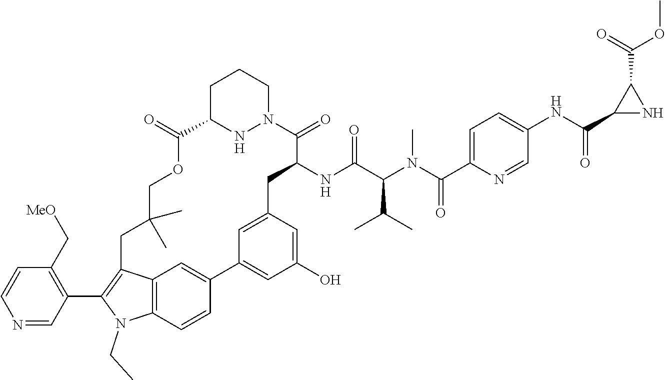 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC(=O)[C@@H]3N[C@H]3C(=O)OC)cn1)C(=O)N1CCC[C@H](N1)C(=O)OCC(C)(C)C2] |
| CB35 | 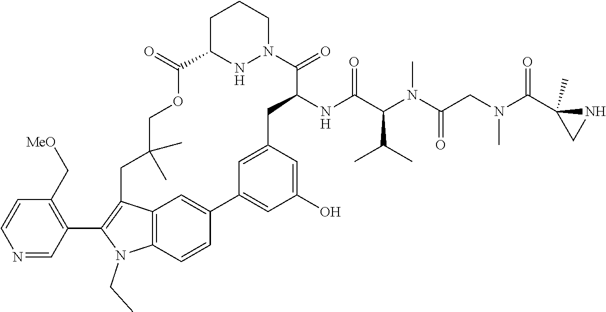 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@]1(C)CN1)C(=O)N1CCC[C@H](N1)C(=O)OCC(C)(C)C2] |
| CB36 | 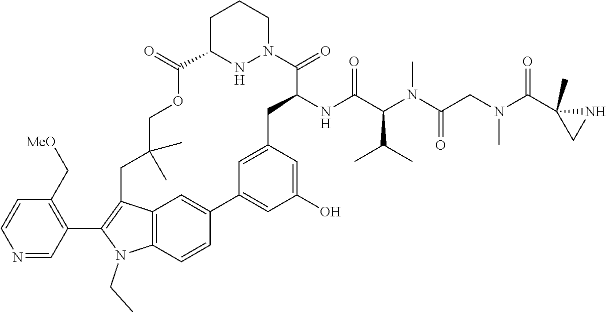 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@]1(C)CN1)C(=O)N1CCC[C@H](N1)C(=O)OCC(C)(C)C2] |
| CB37 | 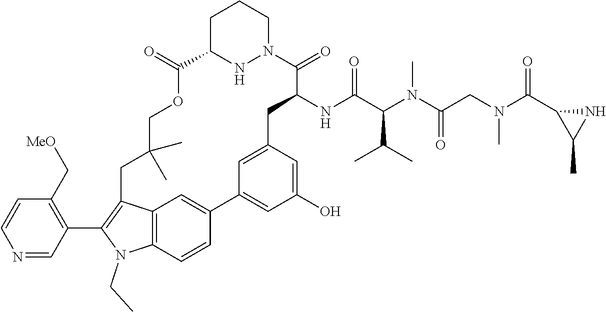 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C)C(=O)N1CCC[C@H](N1)C(=O)OCC(C)(C)C2] |
| CB38 | 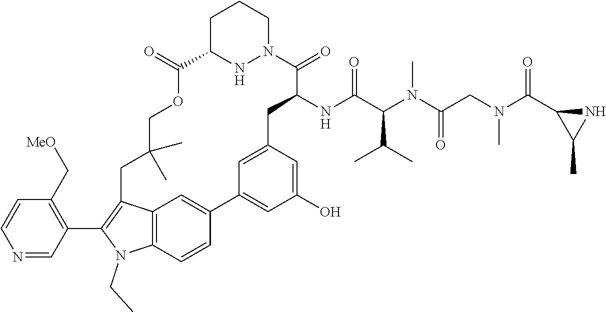 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@H]1C)C(=O)N1CCC[C@H](N1)C(=O)OCC(C)(C)C2] |
| CB40 | 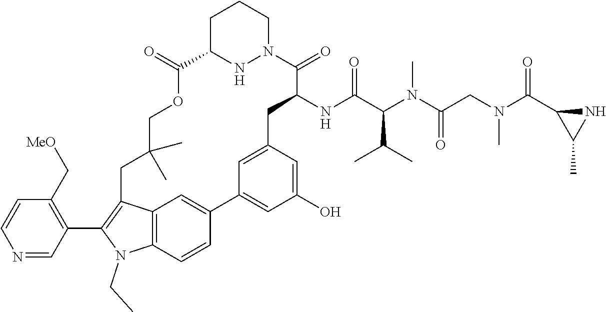 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C)C(=O)N1CCC[C@H](N1)C(=O)OCC(C)(C)C2] |
| CB41 | 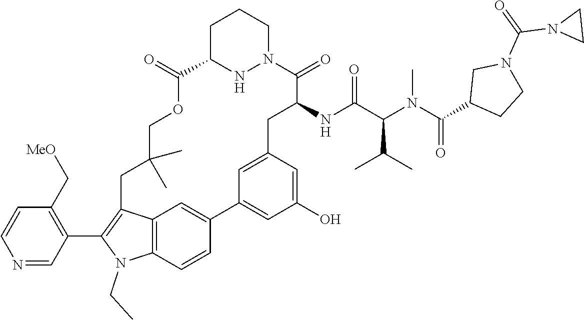 |
| CB42 | 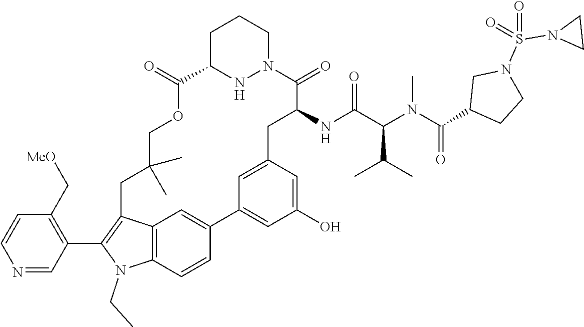 |
| CB43 | 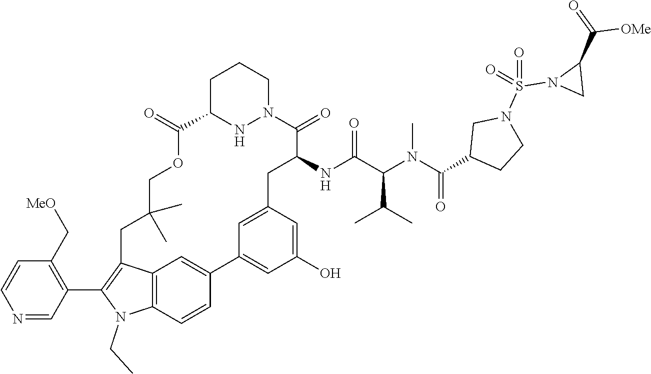 |
| CB44 | 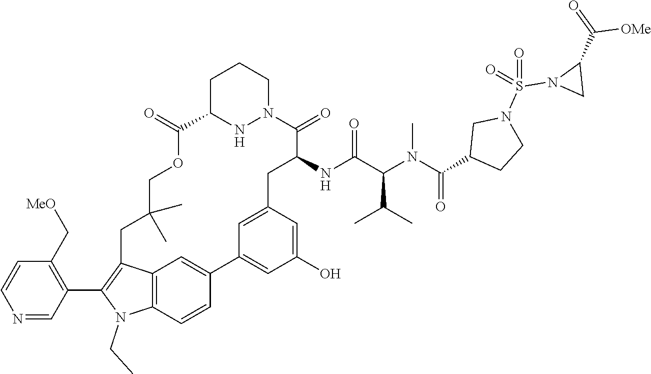 |
| CB46 | 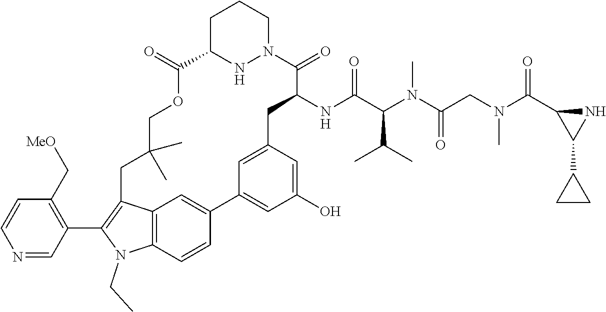 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@H]1N[C@@H]1C1CC1)C(=O)N1CCC[C@H](N1)C(=O)OCC(C)(C)C2] |
| CB48 | 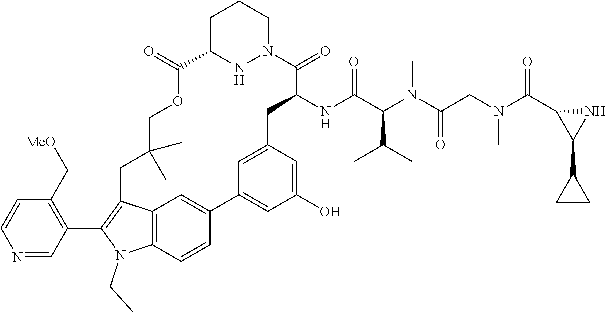 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1C1CC1)C(=O)N1CCC[C@H](N1)C(=O)OCC(C)(C)C2] |
| CB51 | 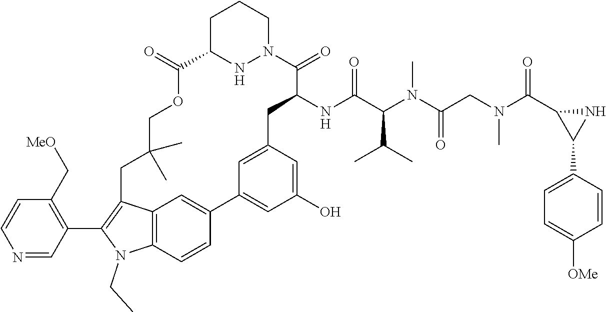 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1c1ccc(OC)cc1)C(=O)N1CCC[C@H](N1)C(=O)OCC(C)(C)C2] |
| CB53 | 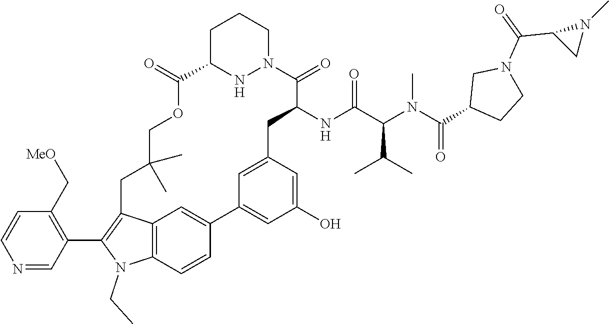 |
| CB54 | 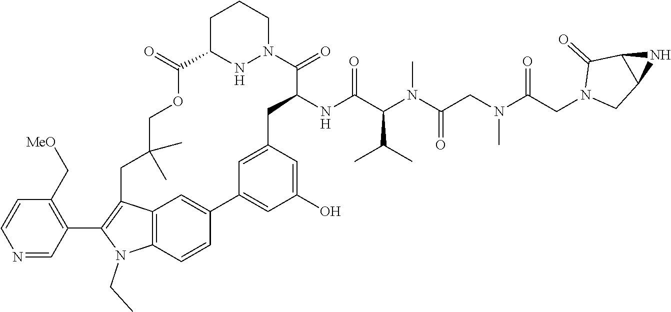 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)CN1C[C@@H]3N[C@@H]3C1=O)C(=O)N1CCC[C@H](N1)C(=O)OCC(C)(C)C2] |
| CB55 | 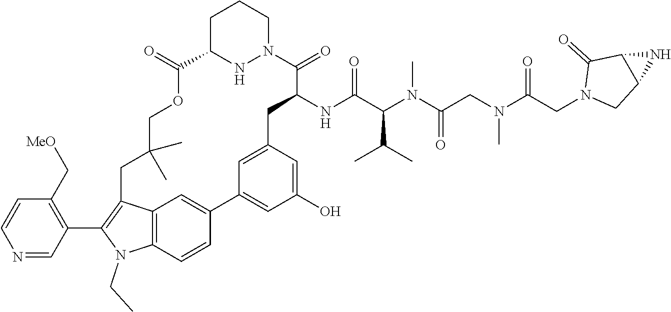 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)CN1C[C@H]3N[C@H]3C1=O)C(=O)N1CCC[C@H](N1)C(=O)OCC(C)(C)C2] |
| CB56 | 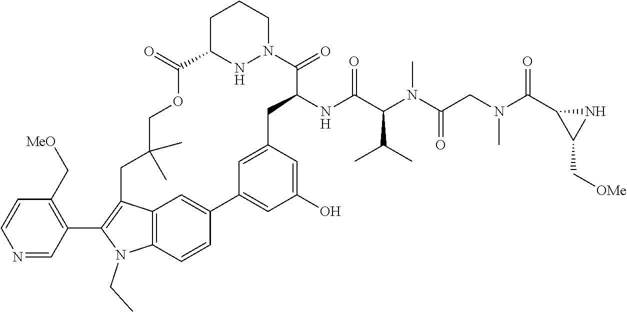 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1COC)C(=O)N1CCC[C@H](N1)C(=O)OCC(C)(C)C2] |
| CB57 | 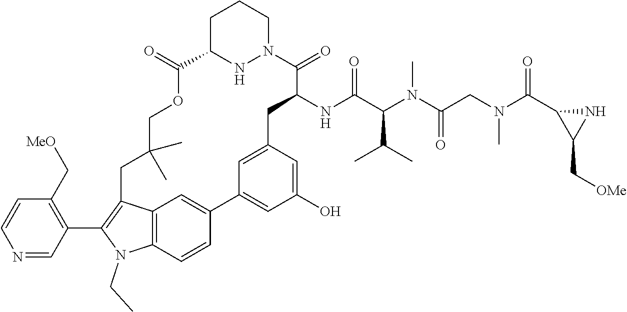 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1COC)C(=O)N1CCC[C@H](N1)C(=O)OCC(C)(C)C2] |
| CB58 | 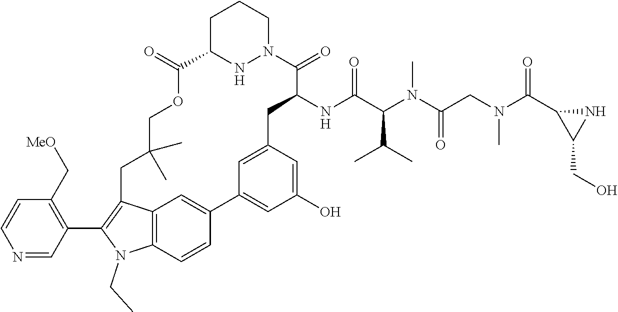 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@@H]1CO)C(=O)N1CCC[C@H](N1)C(=O)OCC(C)(C)C2] |
| CB59 | 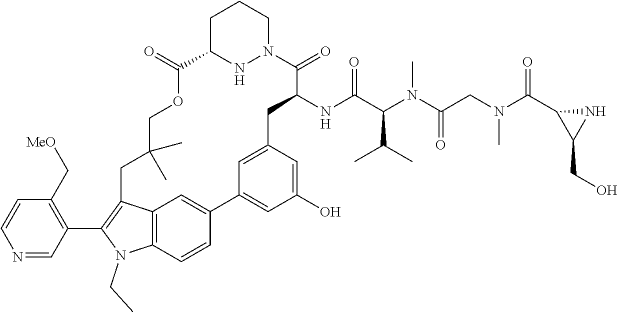 [SMILES: CCn1c(-c2cnccc2COC)c2c3cc(ccc31)-c1cc(O)cc(c1)C[C@H](NC(=O)[C@H](C(C)C)N(C)C(=O)CN(C)C(=O)[C@@H]1N[C@H]1CO)C(=O)N1CCC[C@H](N1)C(=O)OCC(C)(C)C2] |
| CB60 | 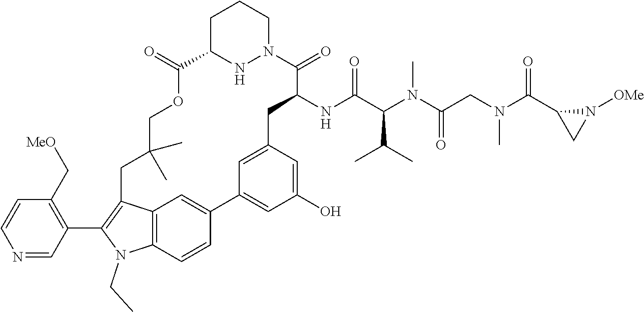 |
| CB61 | 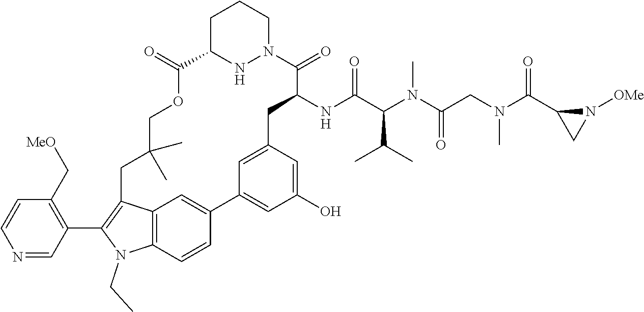 |
| CB62 | 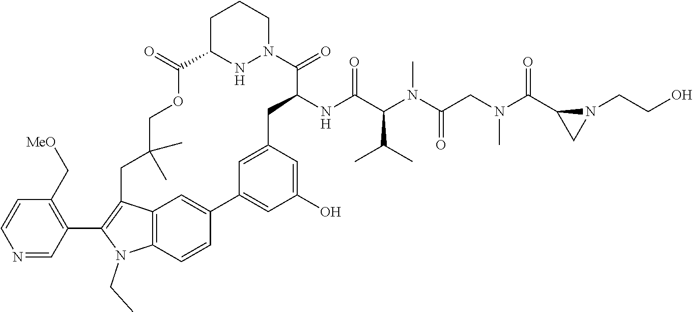 |
| CB63 | 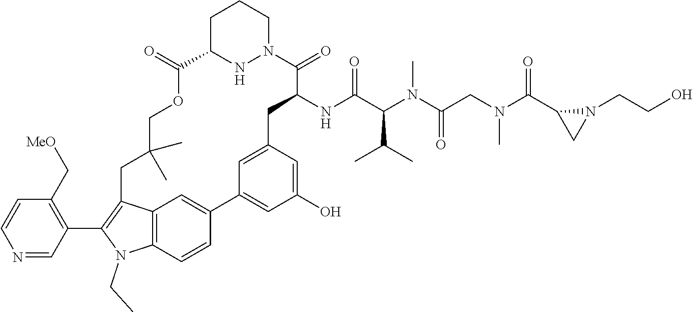 |
| CB73 | 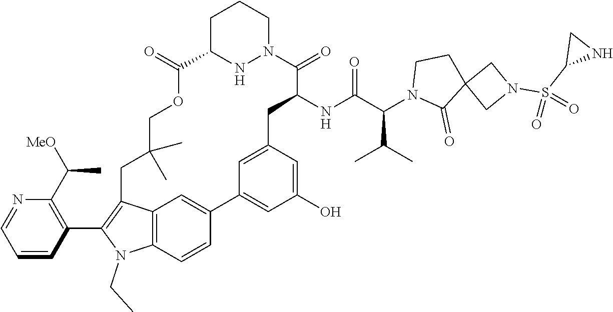 |
| CB74 | 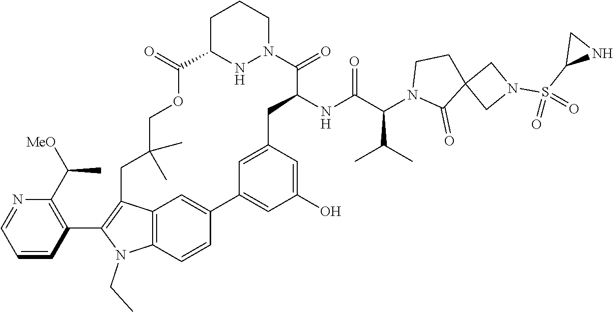 |
| CB76 | 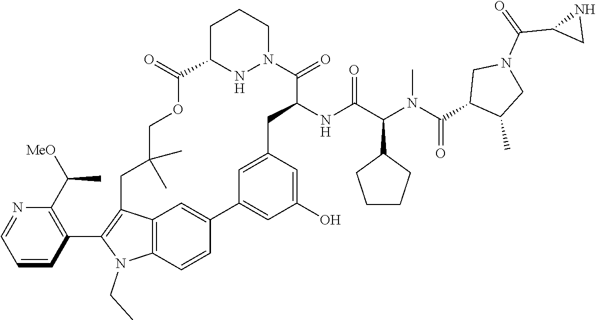 |
| CB78 | 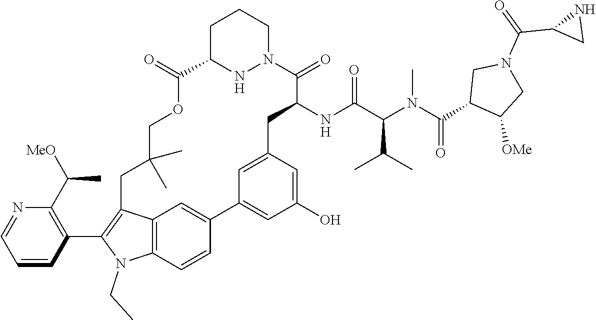 |
| CB79 | 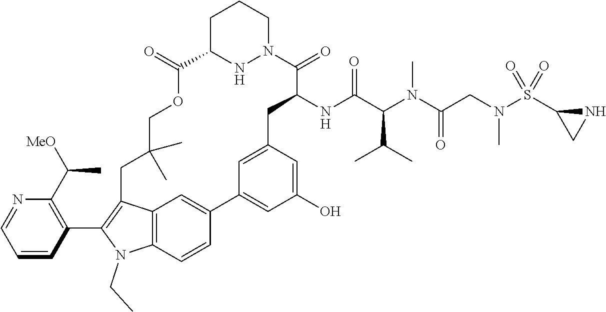 |
| CB80 | 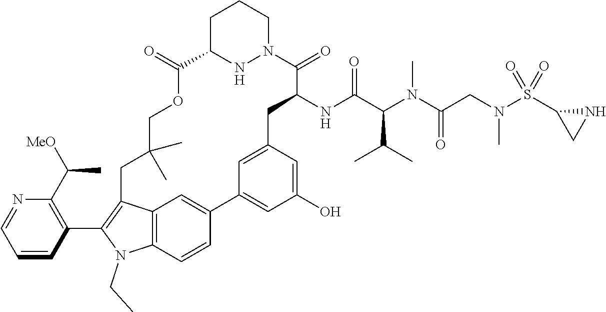 |
| CB83 | 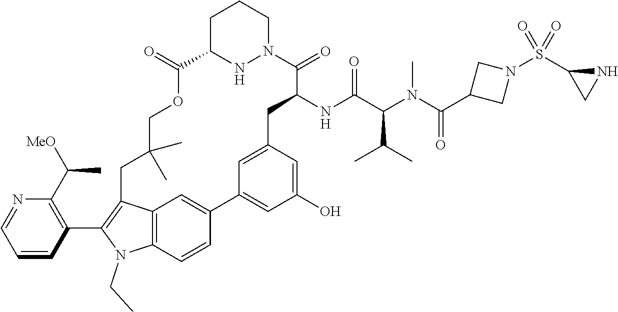 |
| CB84 | 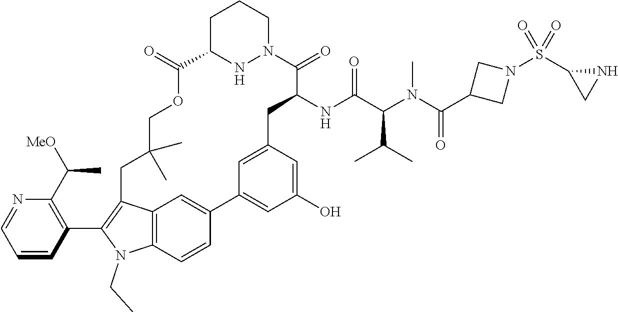 |
| CB87 | 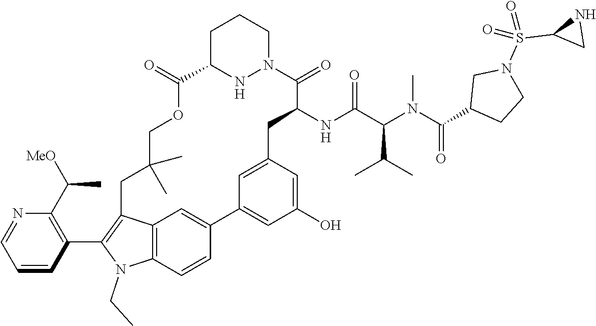 |
| CB88 | 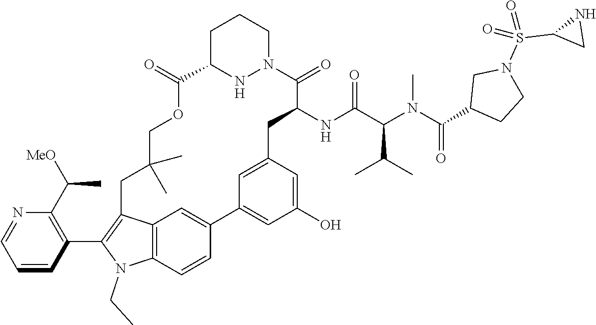 |
| CB91 | 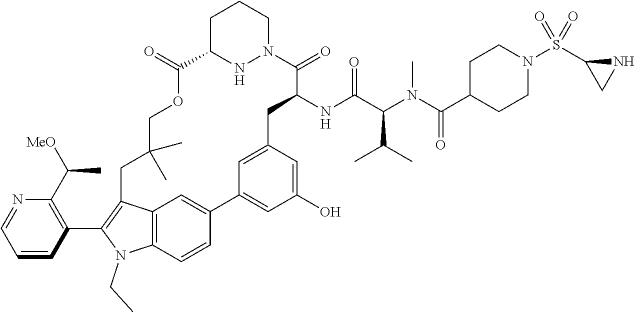 |
| CB92 | 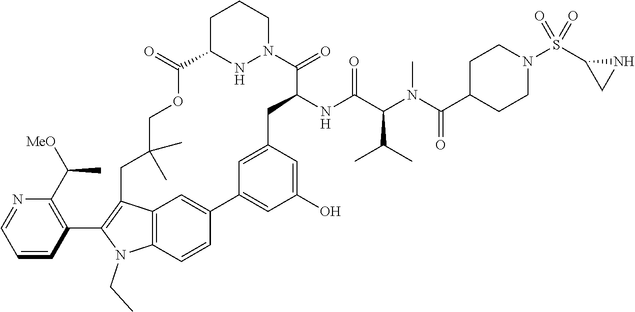 |
| CB97 | 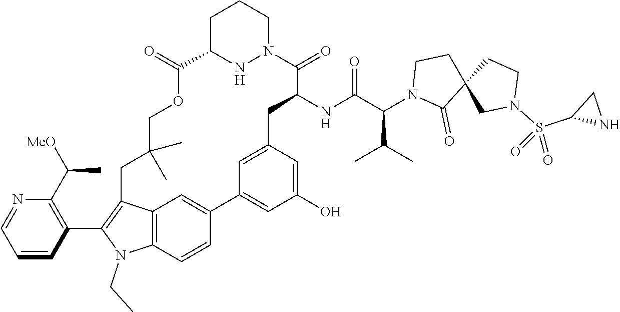 |
| CB98 | 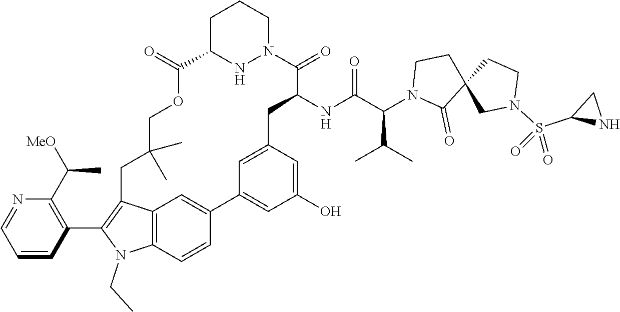 |
| CB101 | 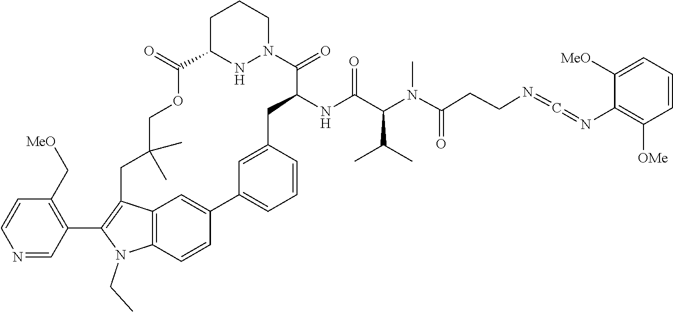 [SMILES: CCn1c(-c2cnccc2COC)c2c3cc(ccc31)-c1cccc(c1)C[C@H](NC(=O)[C@H](C(C)C)N(C)C(=O)CCN=C=Nc1c(OC)cccc1OC)C(=O)N1CCC[C@H](N1)C(=O)OCC(C)(C)C2] |
| CB108 | 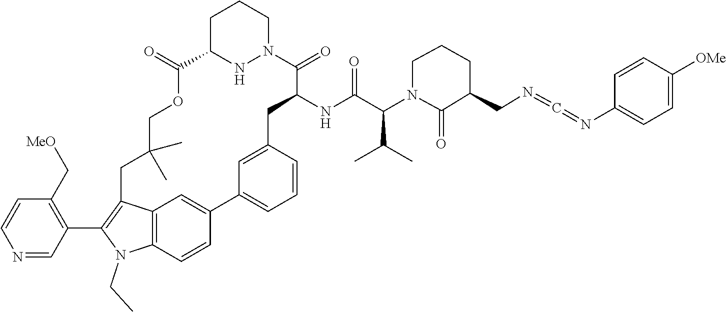 [SMILES: CCn1c(-c2cnccc2COC)c2c3cc(ccc31)-c1cccc(c1)C[C@H](NC(=O)[C@H](C(C)C)N1CCC[C@@H](CN=C=Nc3ccc(OC)cc3)C1=O)C(=O)N1CCC[C@H](N1)C(=O)OCC(C)(C)C2] |
| CB109 | 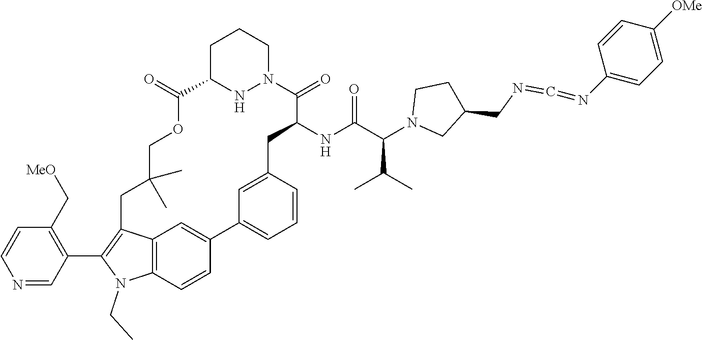 [SMILES: CCn1c(-c2cnccc2COC)c2c3cc(ccc31)-c1cccc(c1)C[C@H](NC(=O)[C@H](C(C)C)N1CC[C@@H](CN=C=Nc3ccc(OC)cc3)C1)C(=O)N1CCC[C@H](N1)C(=O)OCC(C)(C)C2] |
| CB113 | 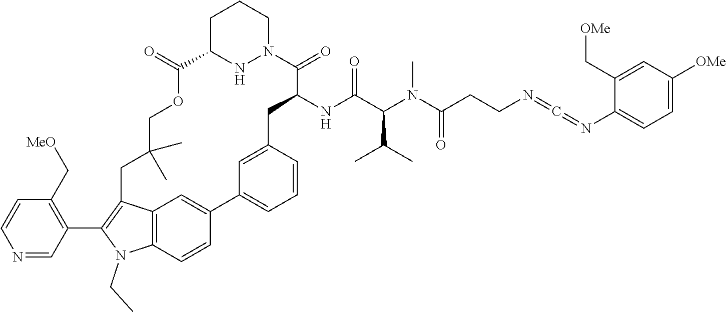 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1COC)C(=O)N1CCC[C@H](N1)C(=O)OCC(C)(C)C2] |
| CB116 | 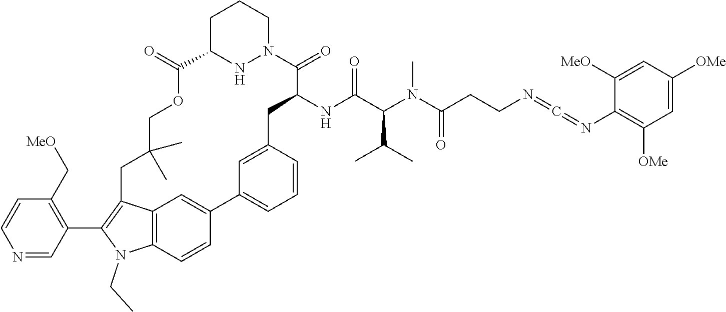 [SMILES: CCn1c(-c2cnccc2COC)c2c3cc(ccc31)-c1cccc(c1)C[C@H](NC(=O)[C@H](C(C)C)N(C)C(=O)CCN=C=Nc1c(OC)cc(OC)cc1OC)C(=O)N1CCC[C@H](N1)C(=O)OCC(C)(C)C2] |
| CB117 | 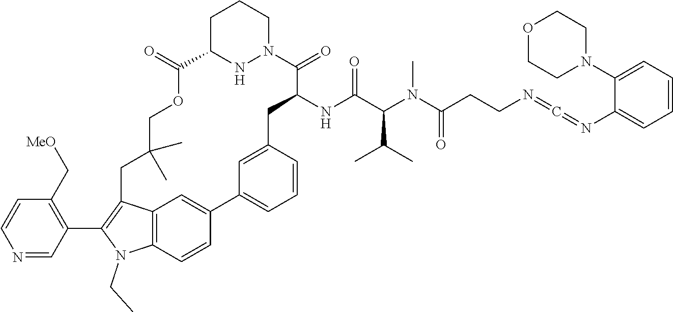 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccccc1N1CCOCC1)C(=O)N1CCC[C@H](N1)C(=O)OCC(C)(C)C2] |
| CB118 | 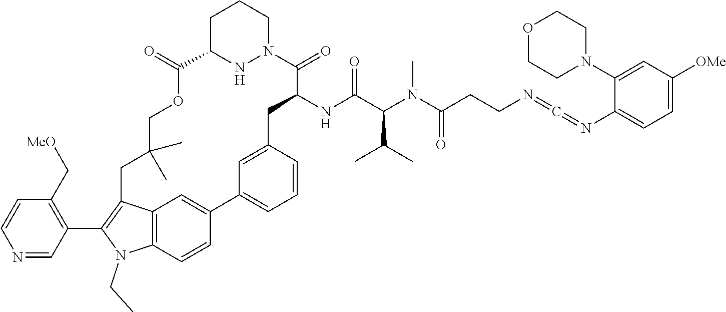 [SMILES: CCn1c(-c2cnccc2COC)c2c3cc(ccc31)-c1cccc(c1)C[C@H](NC(=O)[C@H](C(C)C)N(C)C(=O)CCN=C=Nc1ccc(OC)cc1N1CCOCC1)C(=O)N1CCC[C@H](N1)C(=O)OCC(C)(C)C2] |
| CB119 | 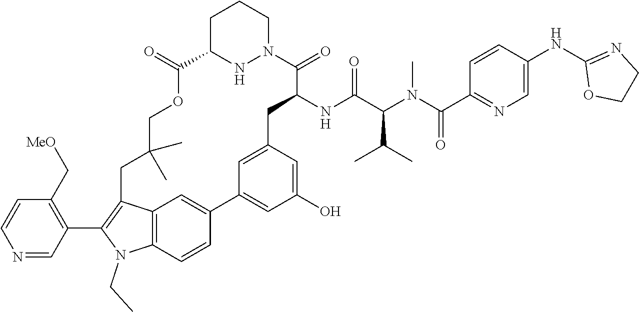 [SMILES: CCn1c(-c2cnccc2COC)c2c3cc(ccc31)-c1cc(O)cc(c1)C[C@H](NC(=O)[C@H](C(C)C)N(C)C(=O)c1ccc(NC3=NCCO3)cn1)C(=O)N1CCC[C@H](N1)C(=O)OCC(C)(C)C2] |
| CB120 | 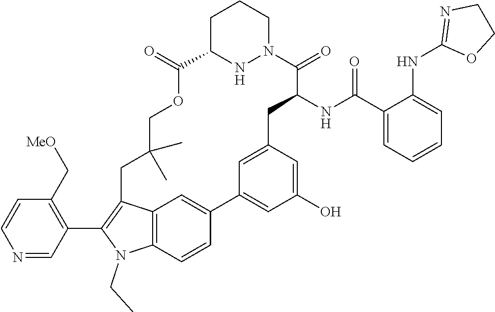 [SMILES: CCn1c(-c2cnccc2COC)c2c3cc(ccc31)-c1cc(O)cc(c1)C[C@H](NC(=O)c1ccccc1NC1=NCCO1)C(=O)N1CCC[C@H](N1)C(=O)OCC(C)(C)C2] |
| CB122 | 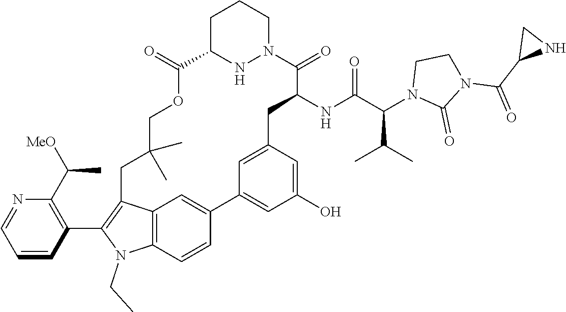 |
| CB123 | 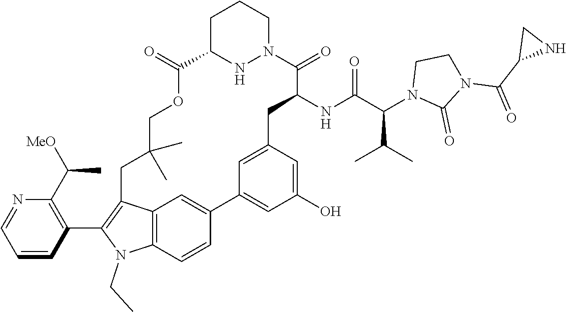 |

Note
that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

In some embodiments, the RAS(ON) inhibitor is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

In some embodiments, the RAS(ON) inhibitor is provided as a conjugate, or salt thereof, comprising the structure of Formula CIV:

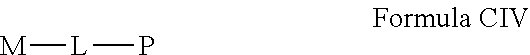

wherein L is a linker;
P is a monovalent organic moiety; and
M has the structure of Formula CVa:

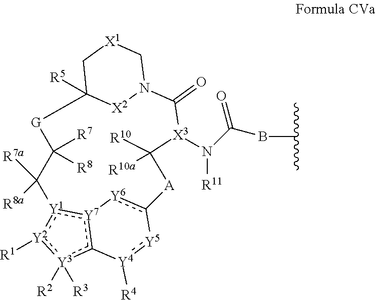

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl.

In some embodiments, the conjugate has the structure of Formula CIV:

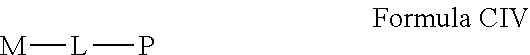

wherein L is a linker;
P is a monovalent organic moiety; and
M has the structure of Formula CVb:

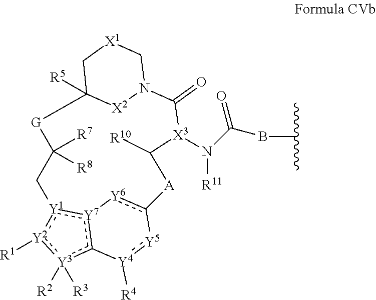

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B is —CH(R⁹)— where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ and Y⁶ are, independently, CH or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹⁰ is hydrogen, hydroxy, C₁-C₃ alkoxy, or C₁-C₃ alkyl; and
R¹¹ is hydrogen or C₁-C₃ alkyl.

In some embodiments, the conjugate has the structure of Formula CIV:

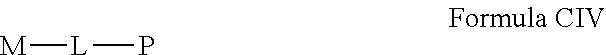

wherein L is a linker;
P is a monovalent organic moiety; and
M has the structure of Formula CVc:

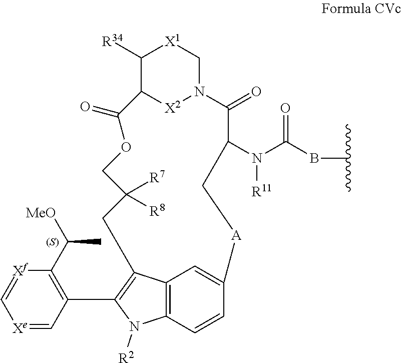

wherein A is optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
X¹ is optionally substituted C₁-C₂ alkylene, NR, O, or S(O)ₙ;
X² is O or NH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
X^{e} and X^{f} are, independently, N or CH;
R² is C₁-C₆ alkyl or 3 to 6-membered cycloalkyl;
R⁷ is C₁-C₃ alkyl;
R⁸ is C₁-C₃ alkyl; and
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R¹¹ is hydrogen or C₁-C₃ alkyl; and
R³⁴ is hydrogen or C₁-C₃ alkyl.

In some embodiments of Formula CI and subformula thereof, X^{e} is N and X^{f} is CH. In some embodiments, X^{e} is CH and X^{f} is N.

In some embodiments, the conjugate has the structure of Formula CIV:

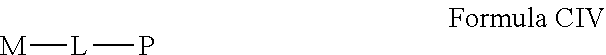

wherein L is a linker;
P is a monovalent organic moiety; and
M has the structure of Formula CVd:

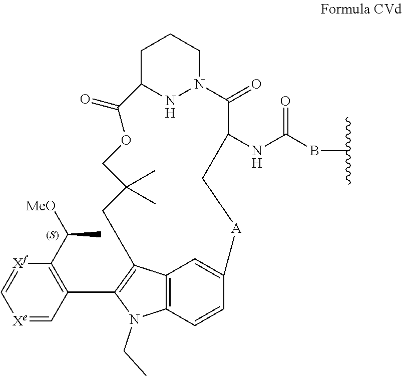

wherein A optionally substituted 3 to 6-membered cycloalkylene, optionally substituted 3 to 6-membered heterocycloalkylene, optionally substituted 6-membered arylene (e.g., phenyl or phenol), or optionally substituted 5 to 6-membered heteroarylene;
B is —CH(R⁹)— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
R⁹ is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl; and
X^{e} and X^{f} are, independently, N or CH.

In some embodiments of Formula CI and subformula thereof, X^{e} is N and X^{f} is CH. In some embodiments, X^{e} is CH and X^{f} is N.

In some embodiments of conjugates of formula CIV, the linker has the structure of Formula CII:

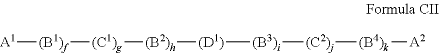

where A¹ is a bond between the linker and B; A² is a bond between P and the linker; B¹, B², B³, and B⁴ each, independently, is selected from optionally substituted C₁-C₂ alkylene, optionally substituted C₁-C₃ heteroalkylene, 0, S, and NR^{N}; R^{N} is hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A². In some embodiments of conjugates of the present invention, the linker is bound to the monovalent organic moiety through a bond to a carboxyl group of an amino acid residue of the monovalent organic moiety.

In some embodiments of conjugates of formula CIV, the monovalent organic moiety is a protein. In some embodiments, the protein is a Ras protein. In some embodiments, the Ras protein is K-Ras G12D or K-Ras G13D.

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below and in WO 2021/091967.

Compounds of Table C1 herein were prepared using methods disclosed herein or were prepared using methods disclosed herein combined with the knowledge of one of skill in the art. Compounds of Table C2 may be prepared using methods disclosed herein or may be prepared using methods disclosed herein combined with the knowledge of one of skill in the art.

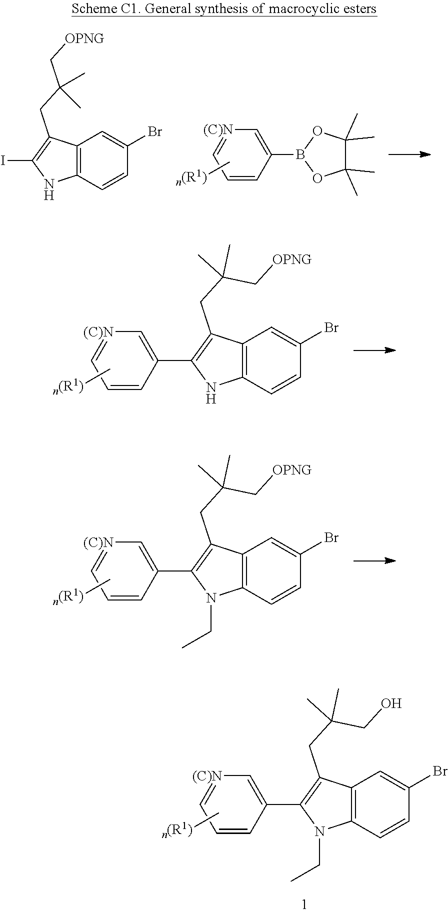

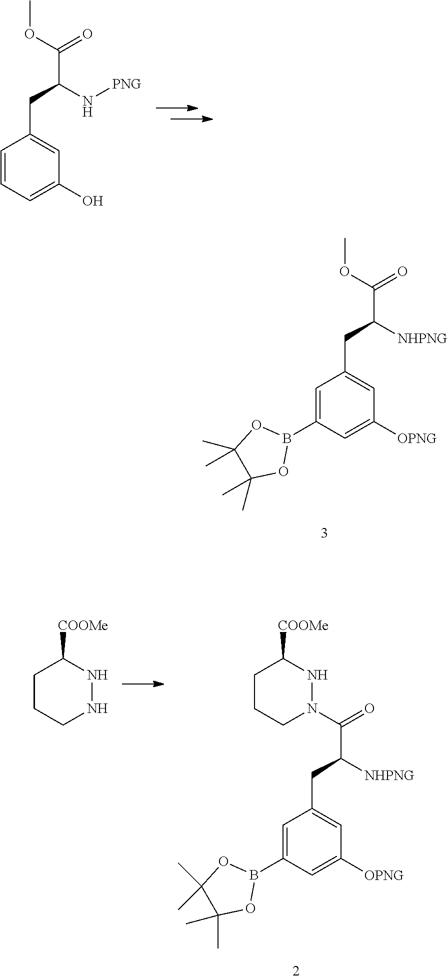

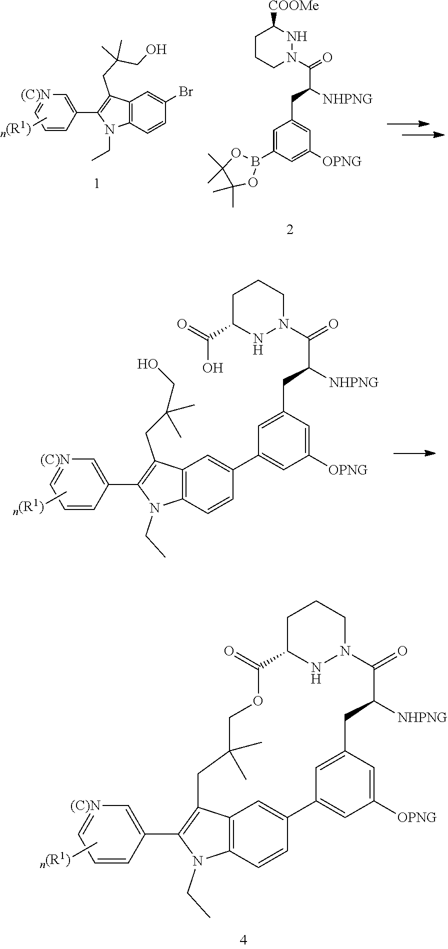

A general synthesis of macrocyclic esters is outlined in Scheme C₁. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions.

Methyl-amino-hexahydropyridazine-3-carboxylate-boronic ester (2) can be prepared in three steps, including protection, iridium catalyst mediated borylation, and coupling with methyl (S)-hexahydropyridazine-3-carboxylate.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of Pd catalyst followed by hydrolysis and macrolactonization steps to result in an appropriately protected macrocyclic intermediate (4). Additional deprotection or functionalization steps are required to produce a final compound. For example, a person of skill in the art would be able to install into a macrocyclic ester a desired —B-L-W group of a compound of Formula (CI), where B, L and Ware defined herein, including by using methods exemplified in certain Schemes below and in the Example section herein.

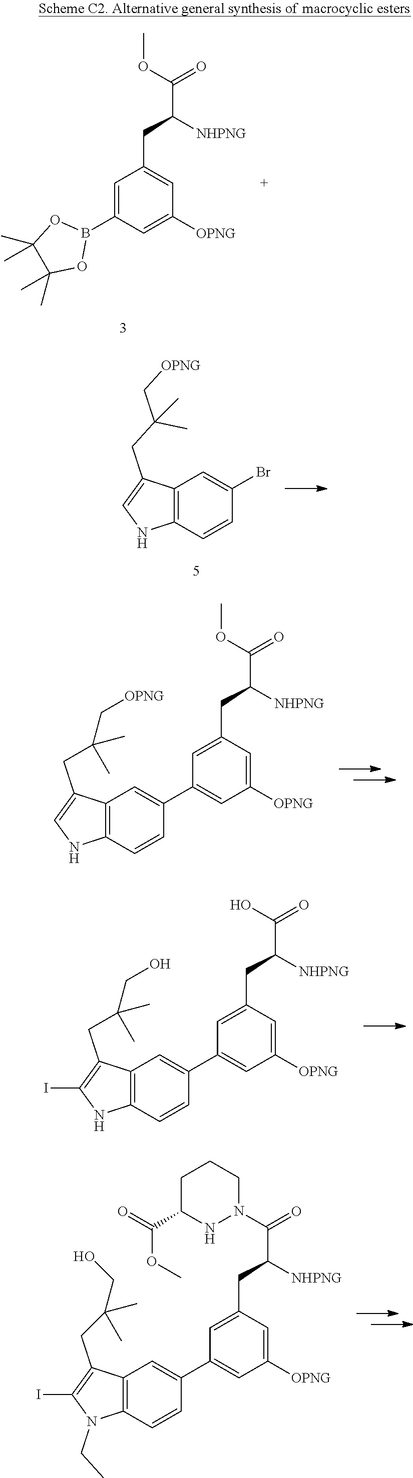

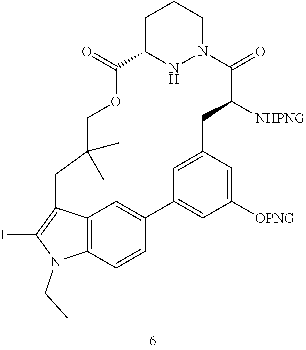

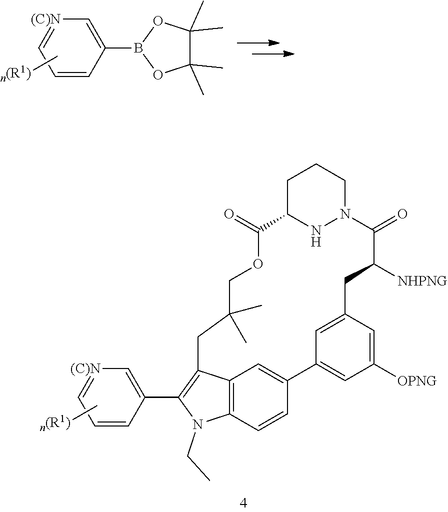

Alternatively, macrocyclic esters can be prepared as described in Scheme C₂. An appropriately protected bromo-indolyl (5) can be coupled in the presence of Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (6). Coupling in the presence of Pd catalyst with an appropriately substituted boronic ester can yield fully a protected macrocycle (4). Additional deprotection or functionalization steps are required to produce a final compound. For example, a person of skill in the art would be able to install into a macrocyclic ester a desired —B-L-W group of a compound of Formula (C₁), where B, L and Ware defined herein, including by using methods exemplified in certain Schemes below and in the Example section herein.

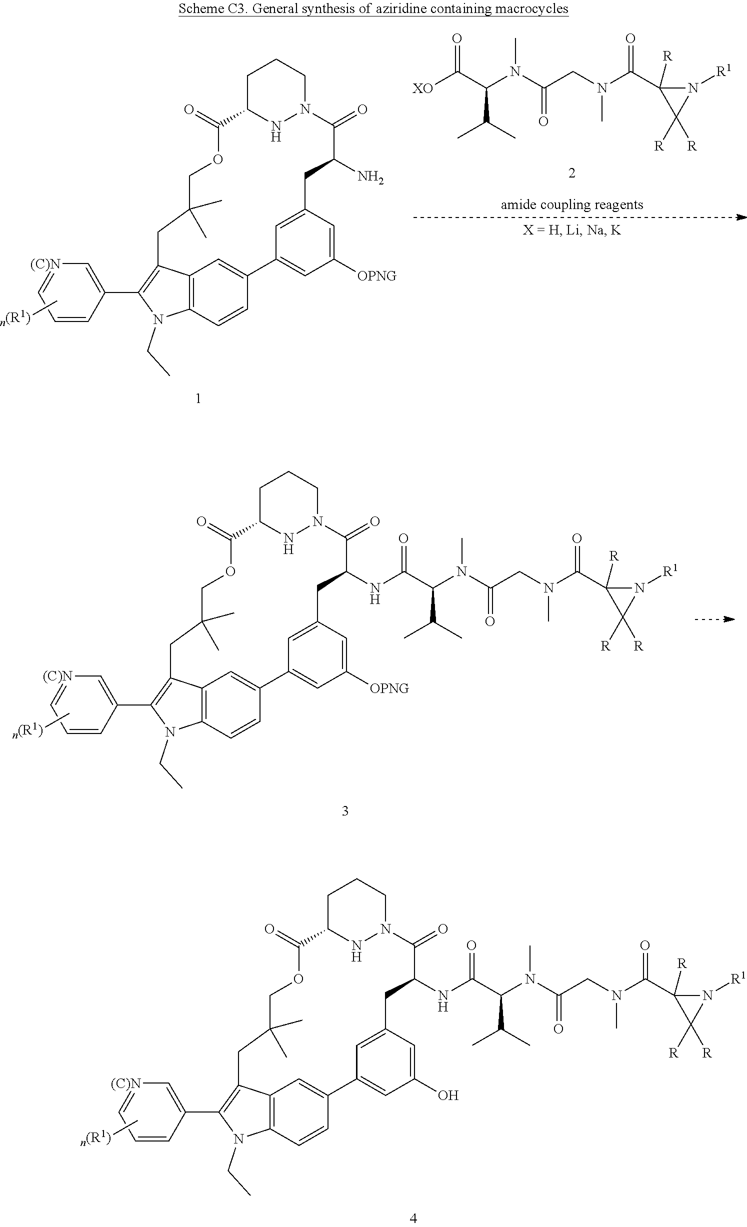

As shown in Scheme C₃, compounds of this type may be prepared by the reaction of an appropriate amine (1) with an aziridine containing carboxylic acid (2) in the presence of standard amide coupling reagents, followed by deprotection of the aziridine, if R¹ is a protecting group, and deprotection of the phenol, if required, to produce the final compound (4).

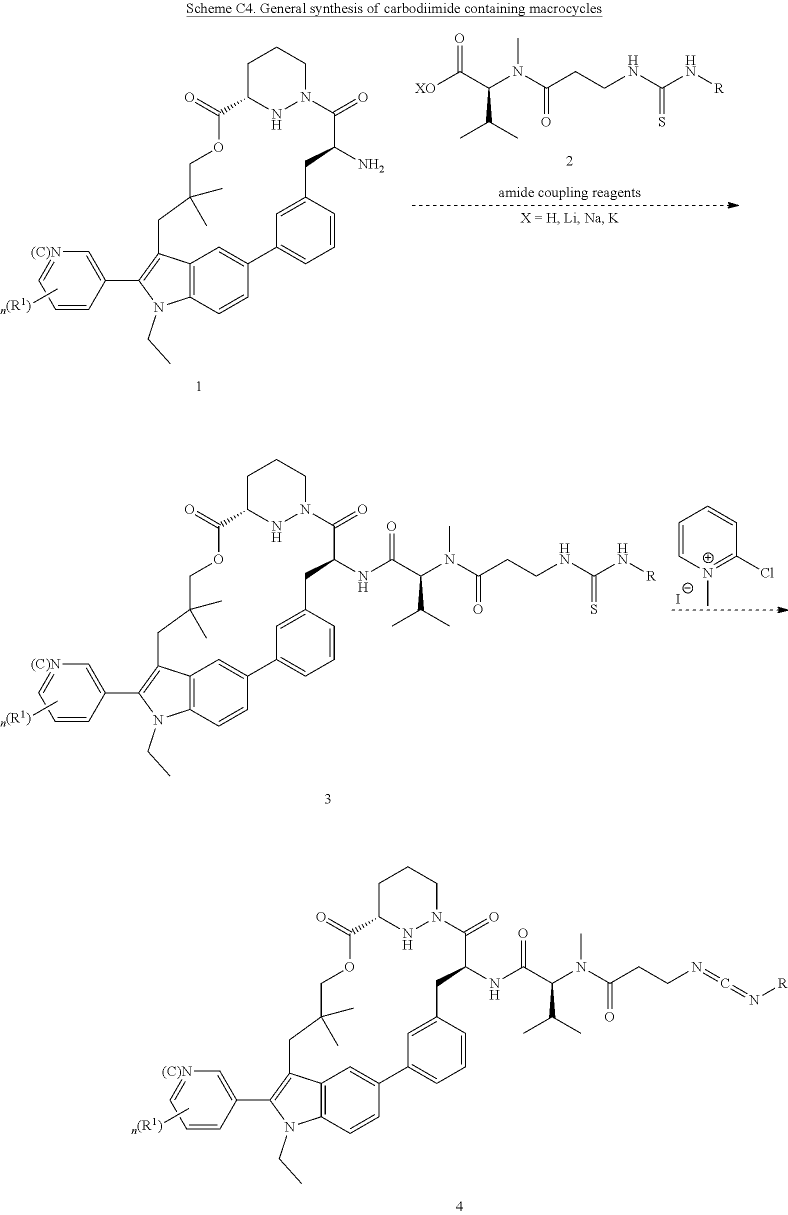

As shown in Scheme C₄, compounds of this type may be prepared by the reaction of an appropriate amine (1) with a thiourea containing carboxylic acid (2) in the presence of standard amide coupling reagents, followed by conversion of the thiourea (3) to a carbodiimide (4) in the presence of 2-chloro-1-methylpyridin-1-ium iodide.

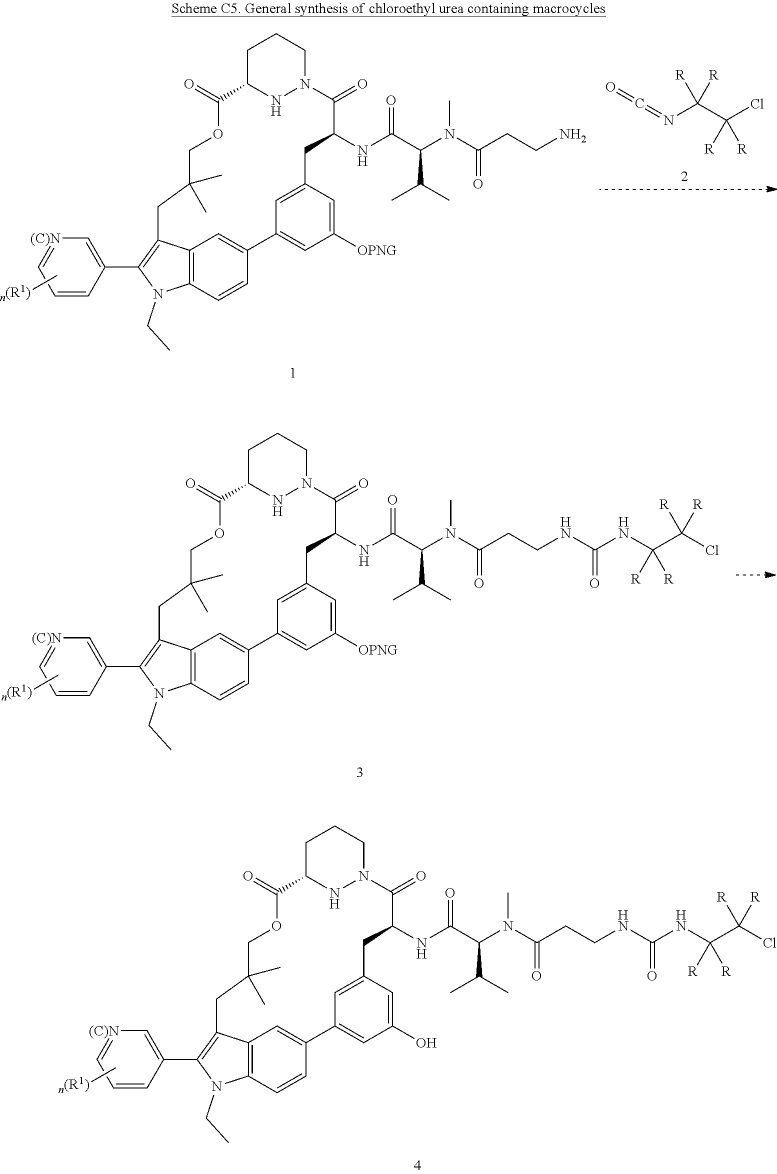

As shown in Scheme C₅, compounds of this type may be prepared by the reaction of an appropriate amine (1) with an isocyanate (2) under basic conditions, followed by deprotection of the phenol, if required, to produce the final compound (4).

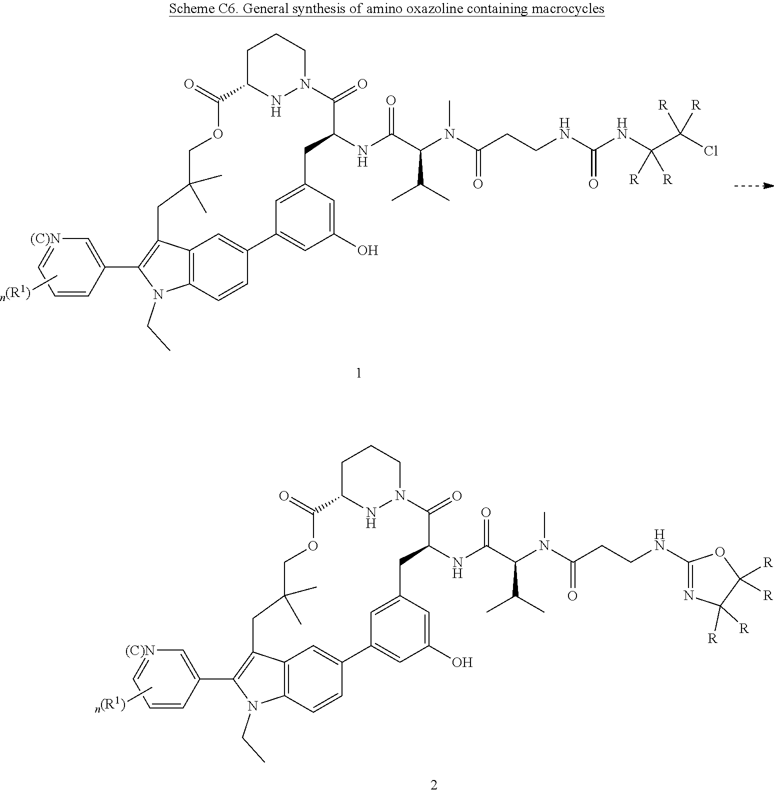

As shown in Scheme 06, compounds of this type may be prepared by cyclization of an appropriate chloroethyl urea (1) under elevated temperatures to produce the final compound (2).

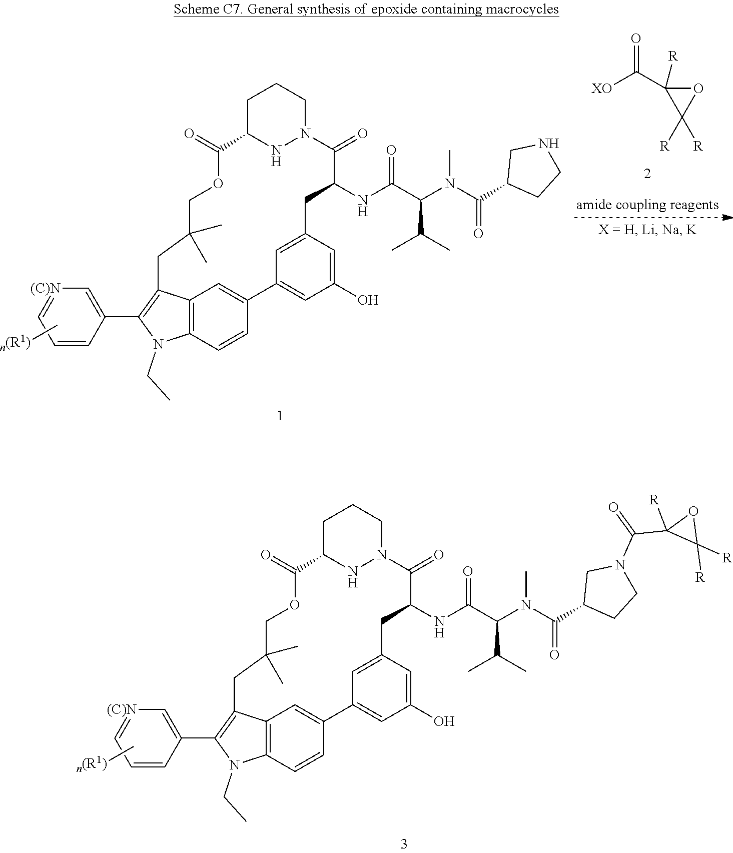

As shown in Scheme C₇, compounds of this type may be prepared by the reaction of an appropriate amine (1) with an epoxide containing carboxylic acid (2) in the presence of standard amide coupling reagents to produce the final compound (3).

In addition, compounds of the disclosure can be synthesized using the methods described in the WO 2021/091967, together with synthetic methods known in the art of synthetic organic chemistry, or variations thereon as appreciated by those skilled in the art. These methods include but are not limited to those methods described in the WO 2021/091967. For example, a person of skill in the art would be able to install into a macrocyclic ester a desired —B-L-W group of a compound of Formula (CI), where B, L and Ware defined herein, including by using methods exemplified in certain Schemes above and in the Example section herein.

In some embodiments, the RAS(ON) inhibitor is a compound, or a pharmaceutically acceptable salt thereof, having the structure of Formula DIa:

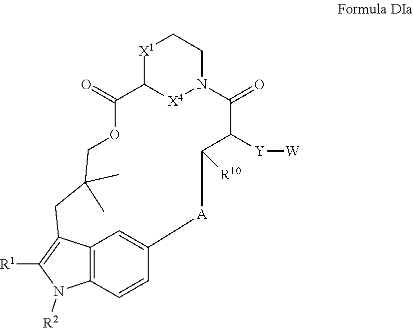

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
Y is

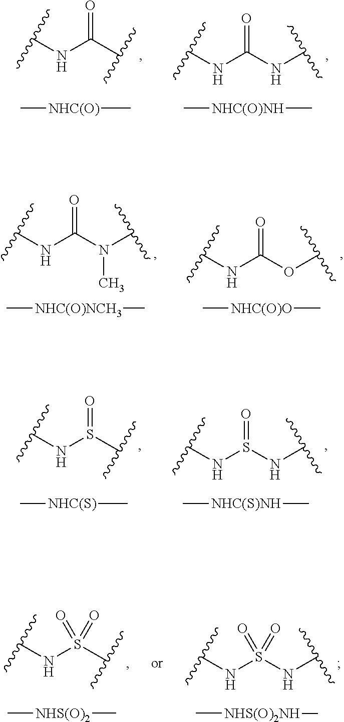

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R¹⁰ is hydrogen, hydroxy, optionally substituted C₁-C₃ alkyl, or optionally substituted C₁-C₆ heteroalkyl.

In some embodiments, the RAS(ON) inhibitor, or pharmaceutically acceptable salt thereof, has

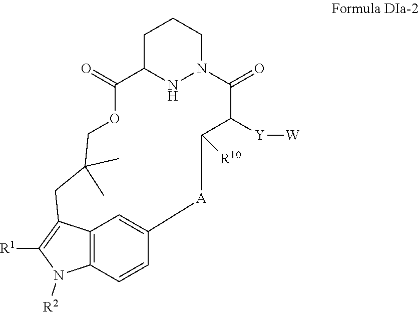

wherein A is optionally substituted 3 to 6-membered cycloalkylene, optionally substituted 3 to 6-membered heterocycloalkylene, optionally substituted 6-membered arylene, or optionally substituted 5 to 6-membered heteroarylene;
Y is

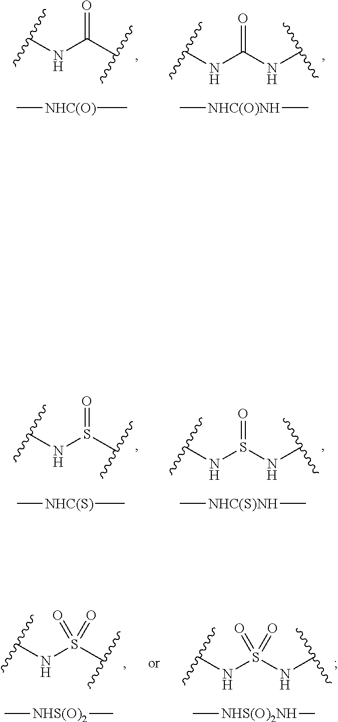

W is hydrogen, C₁-C₄ alkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
R¹⁰ is hydrogen or optionally substituted C₁-C₆ heteroalkyl. In some embodiments, R¹⁰ is hydrogen.

In some embodiments of Formula DIa and subformula thereof, R¹ is optionally substituted 6 to 10-membered aryl or optionally substituted 5 to 10-membered heteroaryl. In some embodiments, R¹ is optionally substituted phenyl or optionally substituted pyridine.

In some embodiments of Formula DIa and subformula thereof, A is optionally substituted thiazole, optionally substituted triazole, optionally substituted morpholino, optionally substituted piperidinyl, optionally substituted pyridine, or optionally substituted phenyl. In some embodiments, A is optionally substituted thiazole, optionally substituted triazole, optionally substituted morpholino, or phenyl. In some embodiments, A is not an optionally substituted phenyl or benzimidazole. In some embodiments, A is not hydroxyphenyl.

In some embodiments of Formula DIa and subformula thereof, Y is —NHC(O)— or —NHC(O)NH—.

In some embodiments, the RAS(ON) inhibitor, or a pharmaceutically acceptable salt thereof, has the structure of Formula DIIa:

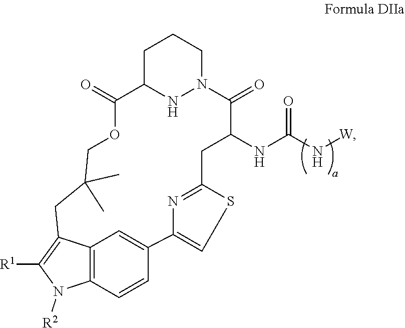

wherein W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
a is 0 or 1.

In some embodiments, the RAS(ON) inhibitor, or a pharmaceutically acceptable salt thereof, has the structure of Formula DIIa-1:

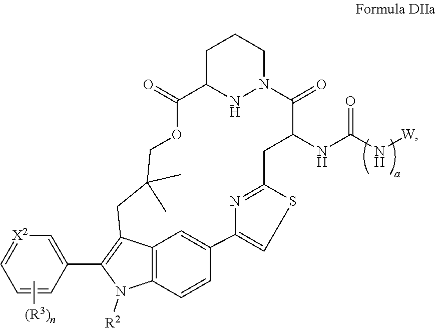

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
   R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
   each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
   n is an integer from 1 to 4.

In some embodiments, the RAS(ON) inhibitor, or a pharmaceutically acceptable salt thereof, has the structure of Formula DIIa-2:

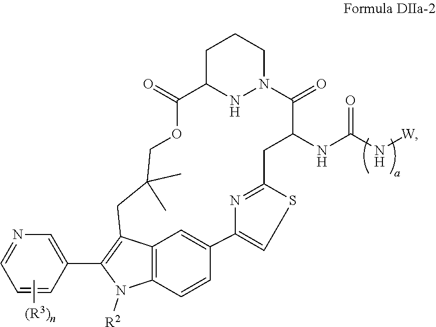

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIa-3:

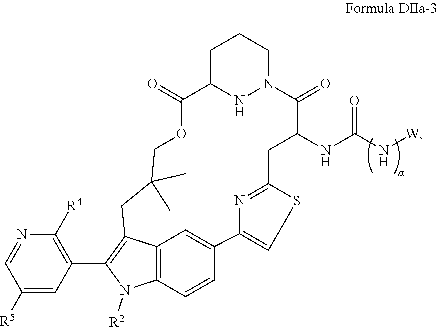

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
   R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
   R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIa-4:

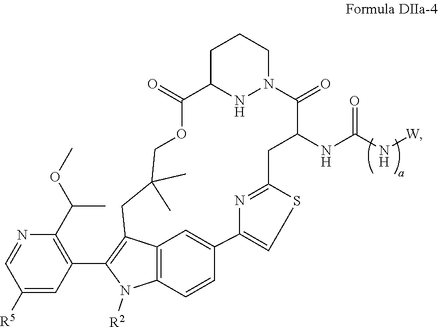

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
   R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
   R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIa-5:

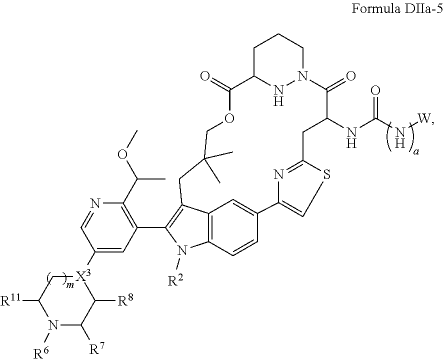

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is H. In some embodiments, X³ is N, m is 1, and R¹¹ is H.

In some embodiments, the RAS(ON) inhibitor, or a pharmaceutically acceptable salt thereof, has the structure of Formula DIIa-6:

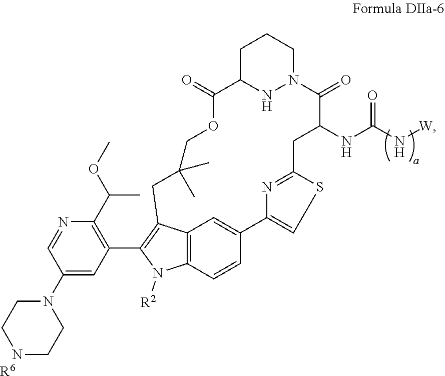

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
   R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
   R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIa-7:

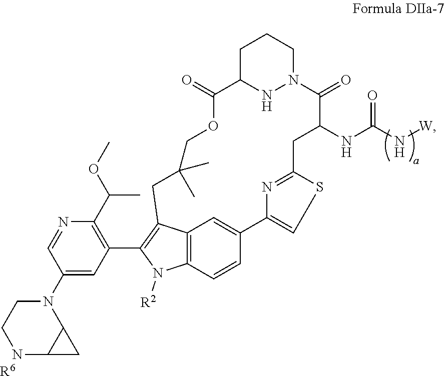

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
   R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and
   R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments (e.g., of any one of Formulae DIIa-6 or DIIa-7), R⁶ is methyl.

In some embodiments, the RAS(ON) inhibitor, or a pharmaceutically acceptable salt thereof, has the structure of Formula DIIa-8 or Formula DIIa-9:

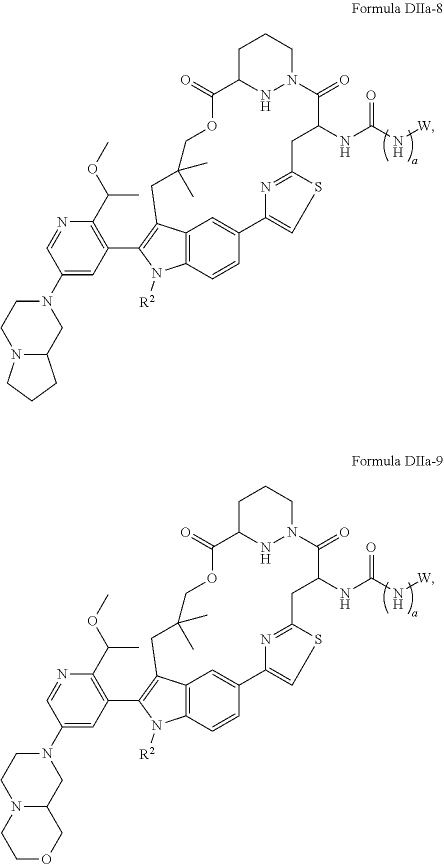

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl.

In some embodiments, the RAS(ON) inhibitor, or a pharmaceutically acceptable salt thereof, has the structure of Formula DIIIa:

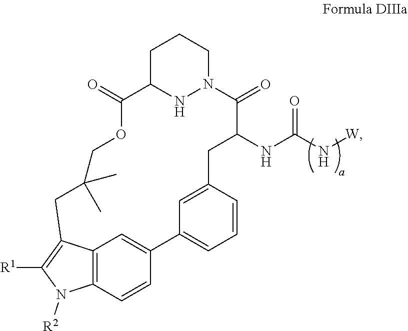

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl.

In some embodiments, the RAS(ON) inhibitor, or a pharmaceutically acceptable salt thereof, has the structure of Formula DIIIa-1:

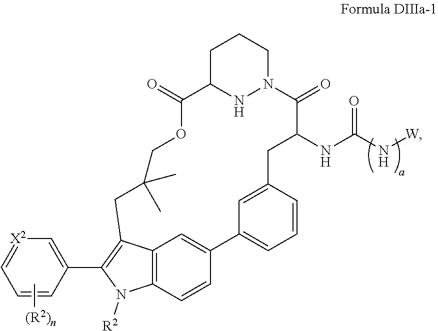

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIIa-2:

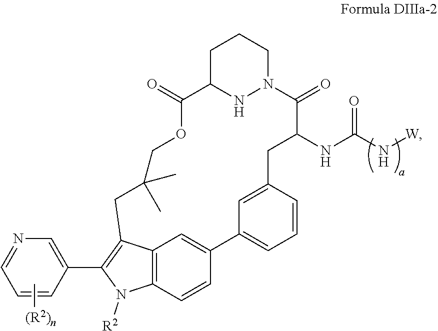

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIIIa-3:

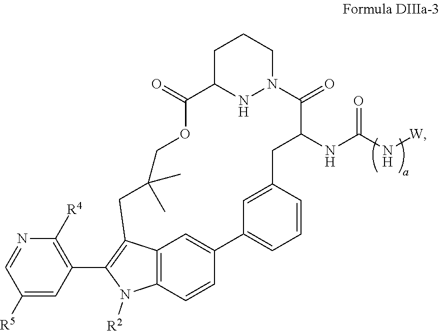

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIIa-4:

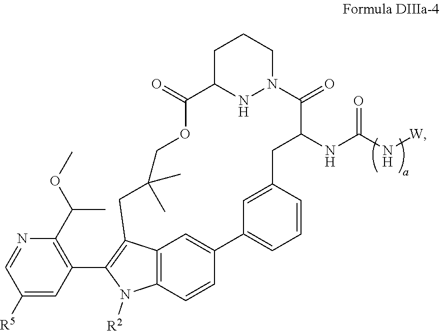

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIIa-5:

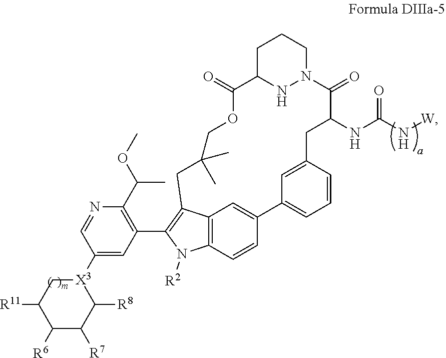

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl;
X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DIIIa-6:

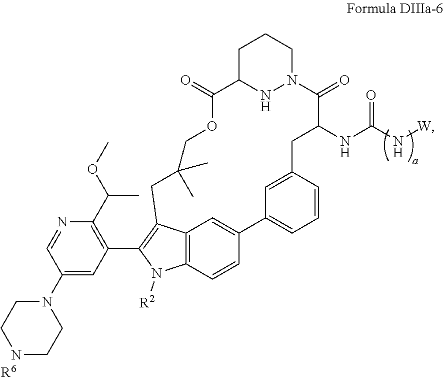

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIIIa-7:

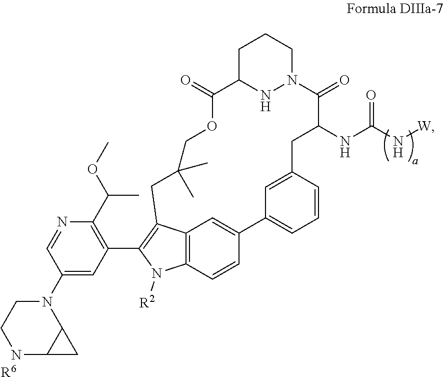

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
R⁶ is hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments (e.g., of any one of Formulae DIIIa-6 or DIIIa-7), R⁶ is methyl.

In some embodiments, the RAS(ON) inhibitor, or a pharmaceutically acceptable salt thereof, has the structure of Formula DIIIa-8 or Formula DIIIa-9:

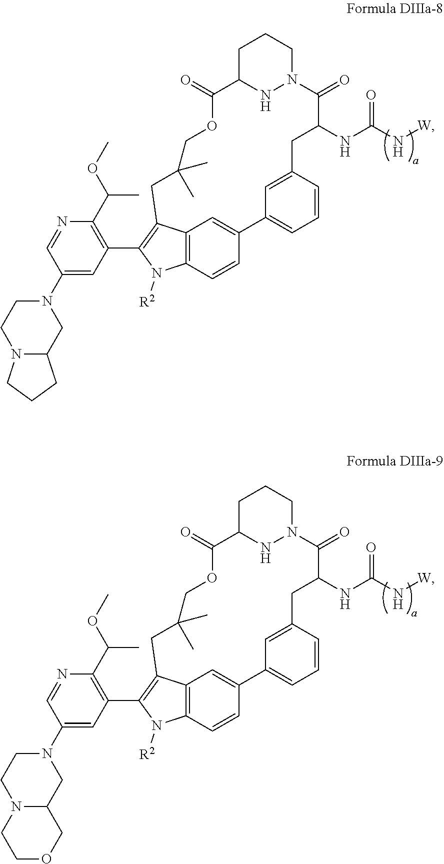

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl.

In some embodiments, the RAS(ON) inhibitor, or a pharmaceutically acceptable salt thereof, has the structure of Formula DIVa:

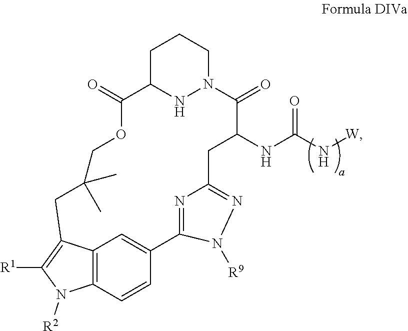

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, or optionally substituted 5 or 6-membered heteroaryl; and
a is 0 or 1.

In some embodiments, the RAS(ON) inhibitor, or a pharmaceutically acceptable salt thereof, has the structure of Formula DIVa-1:

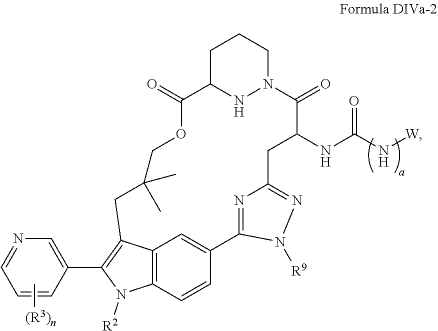

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X² is N or CH.
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl;
R⁹ is H or C₁-C₆ alkyl; and
n is an integer from 1 to 4.

In some embodiments, the RAS(ON) inhibitor, or a pharmaceutically acceptable salt thereof, has the structure of Formula DIVa-2:

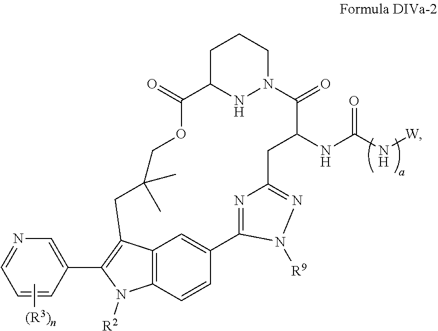

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl;
R⁹ is H or C₁-C₆ alkyl; and
n is an integer from 1 to 4.

In some embodiments, the RAS(ON) inhibitor, or a pharmaceutically acceptable salt thereof, has the structure of Formula DIVa-3:

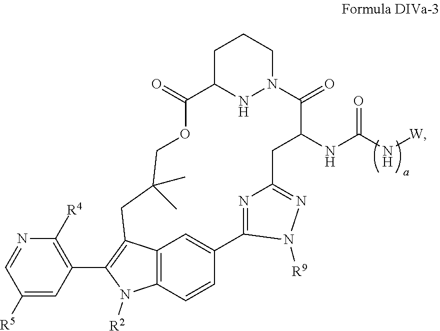

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl; and
R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIVa-4:

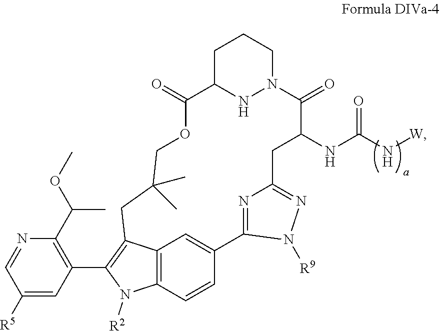

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl; and
R⁵ is halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIVa-5:

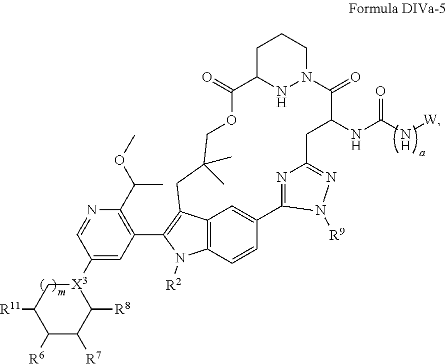

wherein a is 0 or 1;
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X³ is N or CH;
m is 1 or 2;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R⁹ is H or C₁-C₆ alkyl;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DIVa-6:

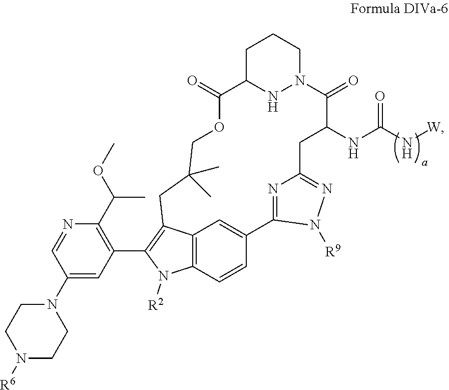

In some embodiments, the RAS(ON) inhibitor, or a pharmaceutically acceptable salt thereof, has the structure of Formula DIVa-7:

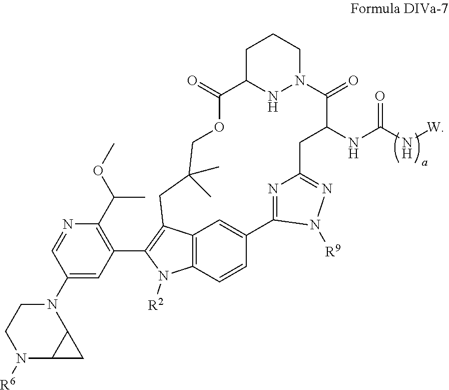

In some embodiments (e.g., of any one of Formulae DIVa-6 or DIVa-7), R⁶ is methyl.

In some embodiments, the RAS(ON) inhibitor, or a pharmaceutically acceptable salt thereof, has the structure of Formula DIVa-8 or Formula DIVa-9:

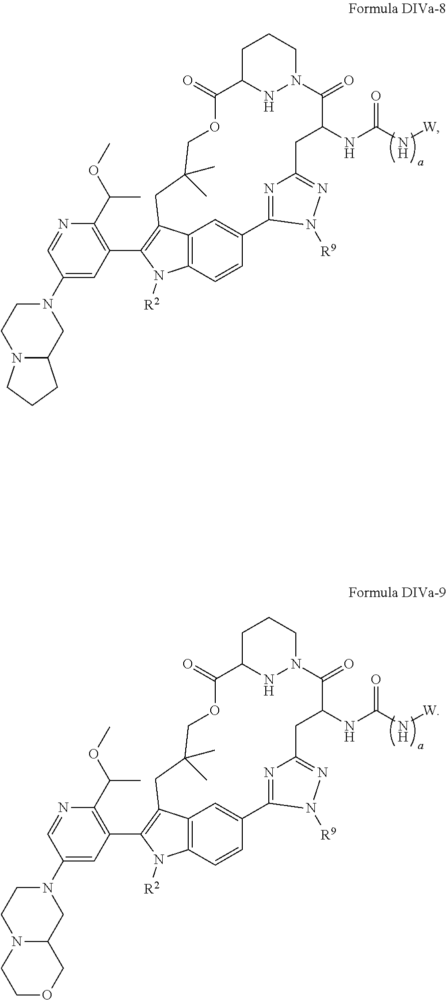

In some embodiments (e.g., of any one of Formulae DIVa, DIVa-1, DIVa-2, DIVa-3, DIVa-4, DIVa-5, DIVa-6, DIVa-7, DIVa-8, or DIVa-9), R⁹ is methyl.

In some embodiments, Y is —NHS(O)₂— or —NHS(O)₂NH—.

In some embodiments, the RAS(ON) inhibitor, or a pharmaceutically acceptable salt thereof, has the structure of Formula DVa:

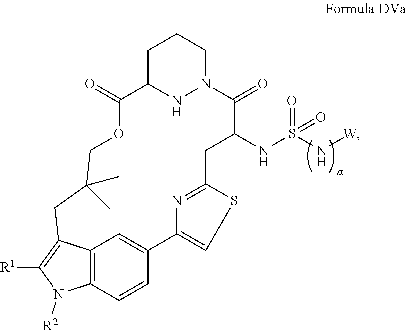

wherein a is 0 or 1.

In some embodiments, the RAS(ON) inhibitor, or a pharmaceutically acceptable salt thereof, has the structure of Formula DVa-1:

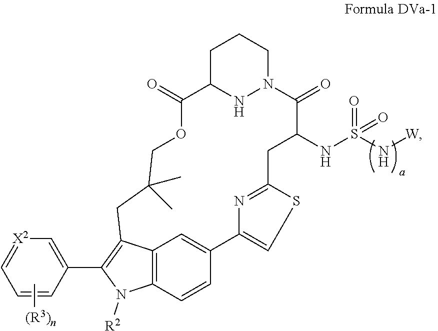

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVa-2:

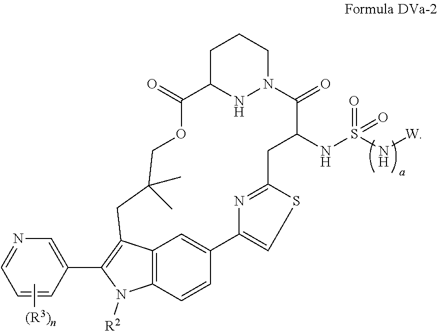

In some embodiments, the RAS(ON) inhibitor, or a pharmaceutically acceptable salt thereof, has the structure of Formula DVa-3:

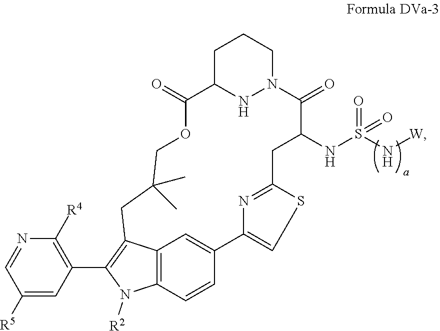

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVa-4:

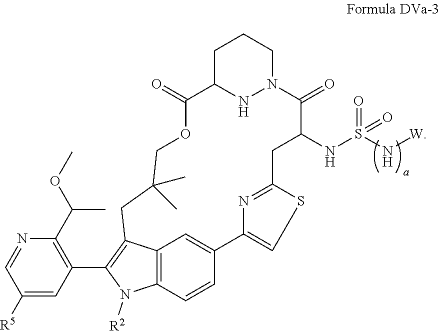

In some embodiments, the RAS(ON) inhibitor, or a pharmaceutically acceptable salt thereof, has the structure of Formula DVa-5:

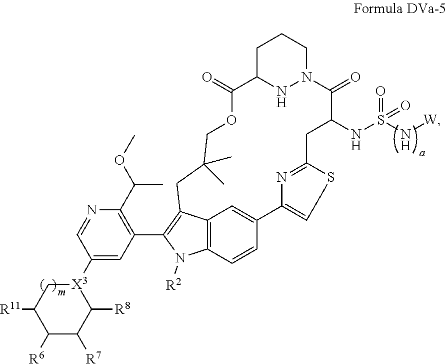

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DVIa:

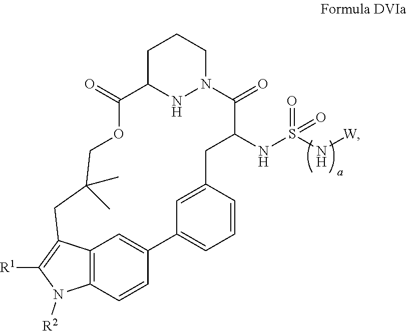

wherein a is 0 or 1.

In some embodiments, the RAS(ON) inhibitor, or a pharmaceutically acceptable salt thereof, has the structure of Formula DVIa-1:

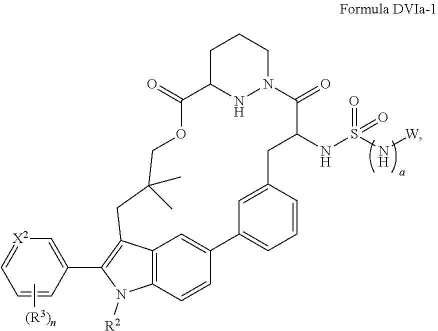

wherein X² is N or CH; each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and n is an integer from 1 to 4.

In some embodiments, the RAS(ON) inhibitor, or a pharmaceutically acceptable salt thereof, has the structure of Formula DVIa-2:

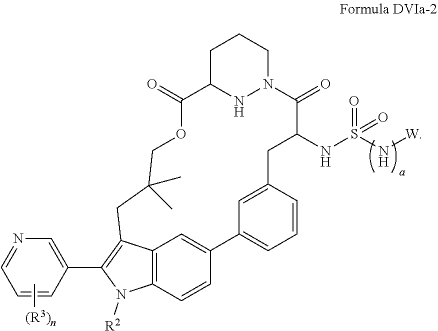

In some embodiments, the RAS(ON) inhibitor, or a pharmaceutically acceptable salt thereof, has the structure of Formula DVIa-3:

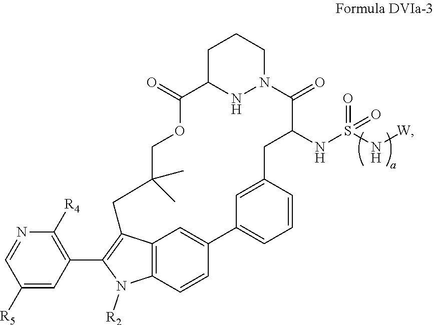

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVIa-4:

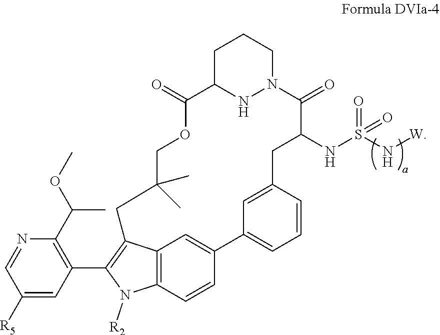

In some embodiments, the RAS(ON) inhibitor, or a pharmaceutically acceptable salt thereof, has the structure of Formula DVIa-5:

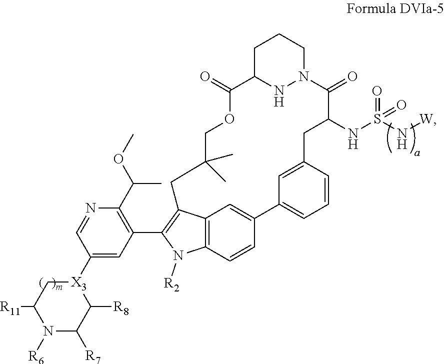

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DVIIa:

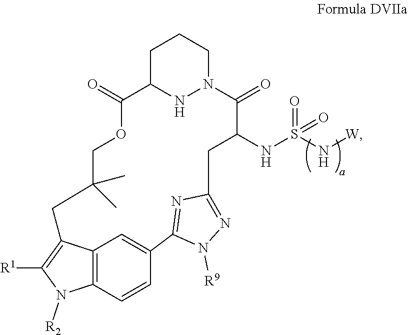

wherein R⁹ is H or C₁-C₆ alkyl; and
a is 0 or 1.

In some embodiments, the RAS(ON) inhibitor, or a pharmaceutically acceptable salt thereof, has the structure of Formula DVIIa-1:

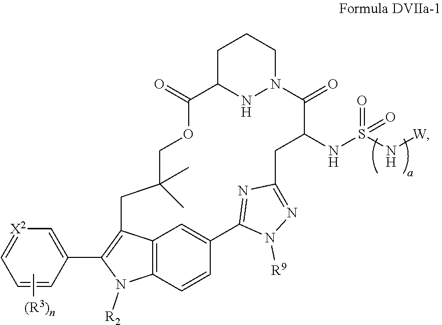

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVIIa-2:

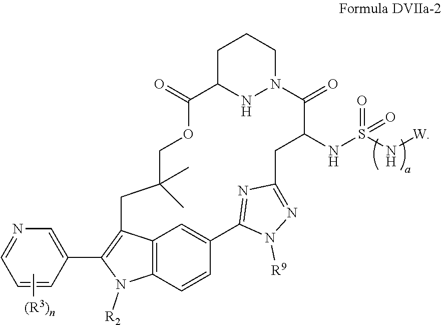

In some embodiments, the RAS(ON) inhibitor, or a pharmaceutically acceptable salt thereof, has the structure of Formula DVIIa-3:

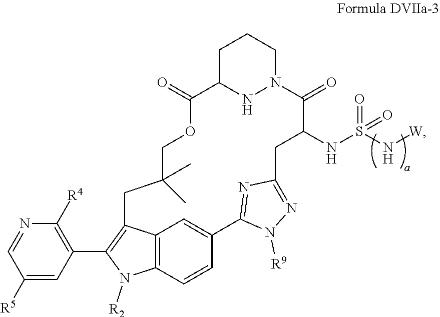

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVIIa-4:

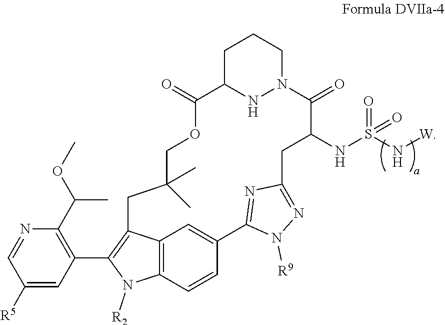

In some embodiments, the RAS(ON) inhibitor, or a pharmaceutically acceptable salt thereof, has the structure of Formula DVIIa-5:

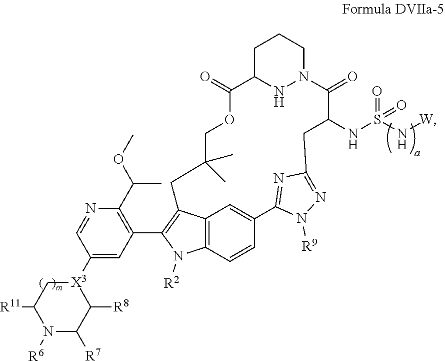

wherein X³ is N or CH;
m is 1 or 2;
R⁶, R⁷, R⁸, and R¹¹ are each independently selected from hydrogren, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae DVIIa, DVIIa-1, DVIIa-2, DVIIa-3, DVIIa-4, or DVIIa-5), R⁹ is methyl.

In some embodiments, Y is —NHS(O)— or —NHS(O)NH—.

In some embodiments, the RAS(ON) inhibitor, or a pharmaceutically acceptable salt thereof, has the structure of Formula DVIIIa:

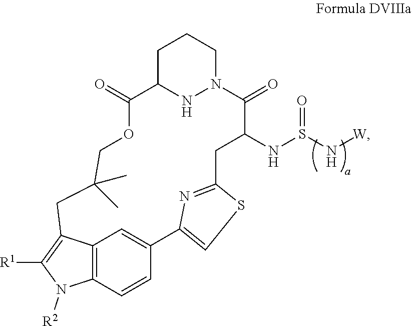

wherein a is 0 or 1.

In some embodiments, the RAS(ON) inhibitor, or a pharmaceutically acceptable salt thereof, has the structure of Formula Villa-1:

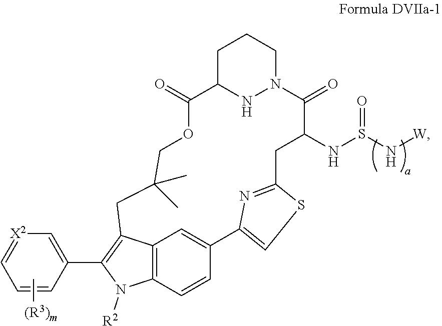

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DVIIIa-2:

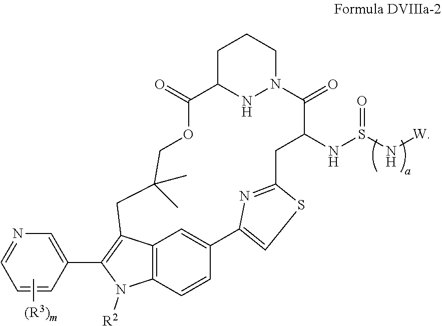

In some embodiments, the RAS(ON) inhibitor, or a pharmaceutically acceptable salt thereof, has the structure of Formula DVIIIa-3:

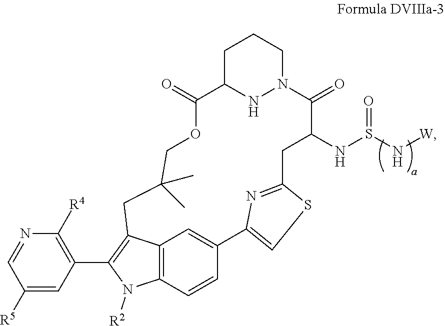

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DVIIIa-4:

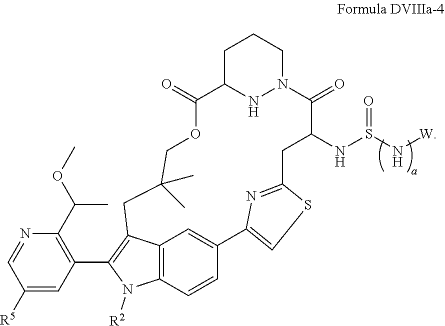

In some embodiments, the RAS(ON) inhibitor, or a pharmaceutically acceptable salt thereof, has the structure of Formula DVIIIa-5:

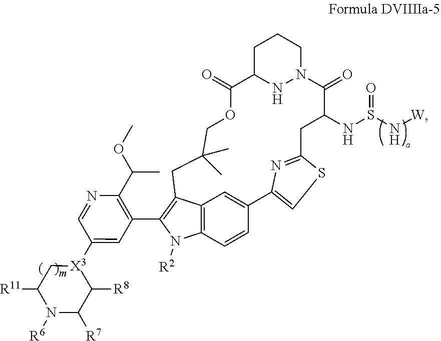

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DIXa:

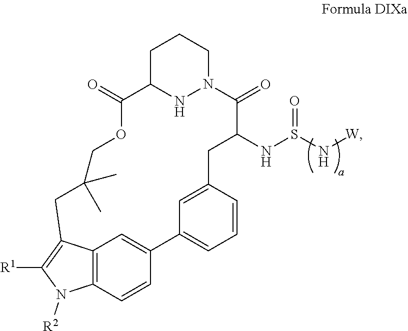

wherein a is 0 or 1.

In some embodiments, the RAS(ON) inhibitor, or a pharmaceutically acceptable salt thereof, has the structure of Formula DIXa-1:

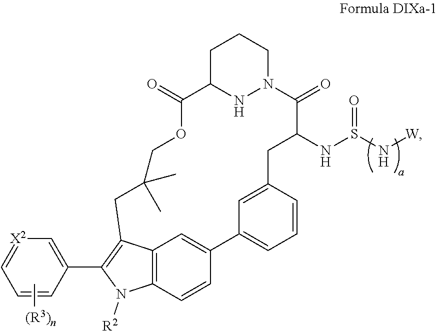

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DIXa-2:

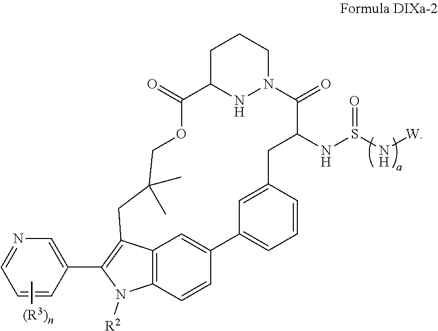

In some embodiments, the RAS(ON) inhibitor, or a pharmaceutically acceptable salt thereof, has the structure of Formula DIXa-3:

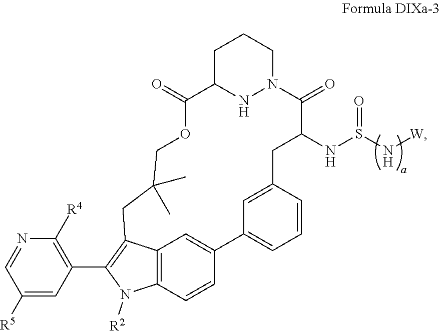

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DIXa-4:

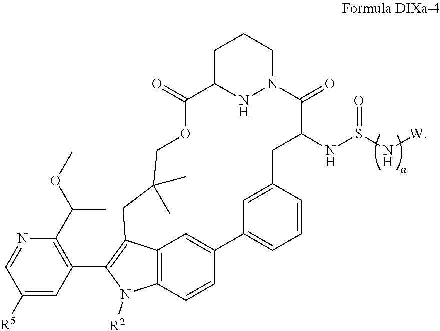

In some embodiments, the RAS(ON) inhibitor, or a pharmaceutically acceptable salt thereof, has the structure of Formula DIXa-5:

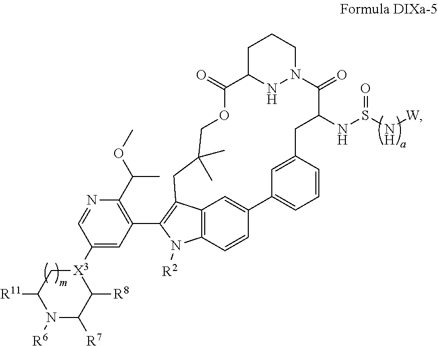

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments, the RAS(ON) inhibitor, or a pharmaceutically acceptable salt thereof, has the structure of Formula DXa:

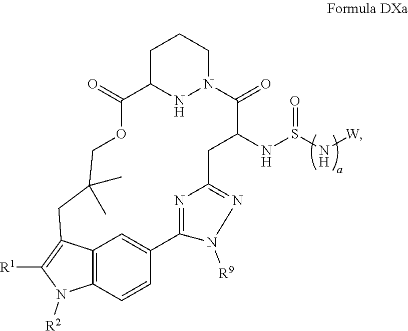

wherein R⁹ is H or C₁-C₆ alkyl; and
a is 0 or 1.

In some embodiments, the RAS(ON) inhibitor, or a pharmaceutically acceptable salt thereof, has the structure of Formula DXa-1:

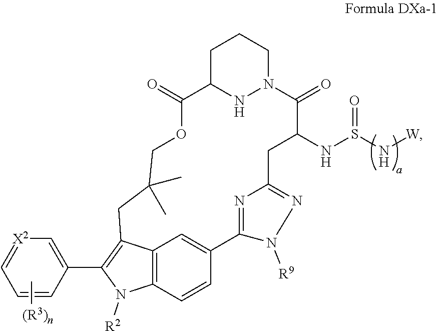

wherein X² is N or CH;
each R³ is independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl; and
n is an integer from 1 to 4.

In some embodiments, the RAS(ON) inhibitor, or a pharmaceutically acceptable salt thereof, has the structure of Formula DXa-2:

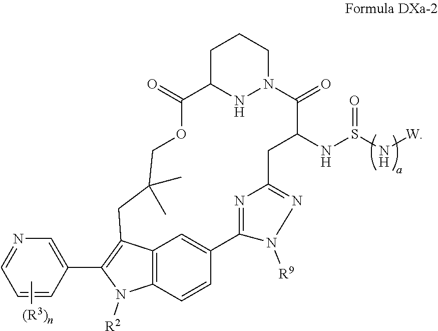

In some embodiments, the RAS(ON) inhibitor, or a pharmaceutically acceptable salt thereof, has the structure of Formula DXa-3:

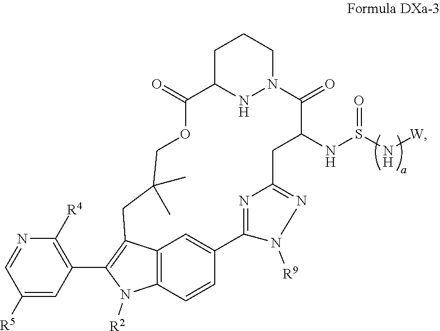

wherein R⁴ and R⁵ are each independently selected from halogen, cyano, hydroxy, optionally substituted amine, optionally substituted amido, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 11-membered heterocycloalkyl (e.g., optionally substituted 3 to 6-membered heterocycloalkyl), optionally substituted 6 to 10-membered aryl, or optionally substituted 5 to 10-membered heteroaryl.

In some embodiments, the RAS(ON) inhibitor, or a pharmaceutically acceptable salt thereof, has the structure of Formula DXa-4:

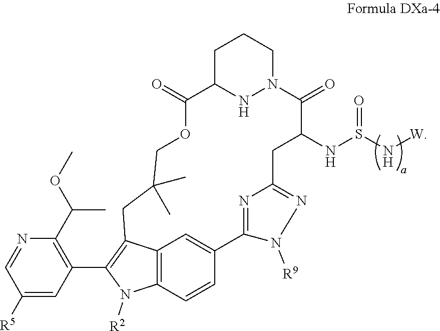

In some embodiments, the RAS(ON) inhibitor, or a pharmaceutically acceptable salt thereof, has the structure of Formula DXa-5:

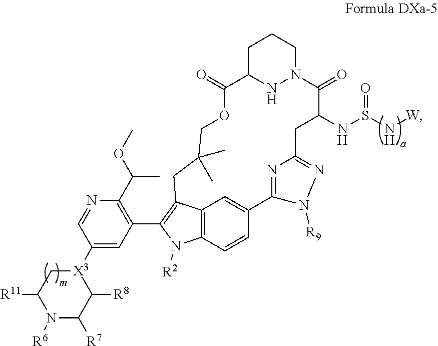

wherein X³ is N or CH;
m is 1 or 2;
R⁶, R⁷, R⁸, and R¹¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R⁶ and R⁷ combine with the atoms to which they are attached to form an optionally substituted 3 to 8-membered cycloalkyl or an optionally substituted 3 to 8-membered heterocycloalkyl; or
R⁷ and R⁸ combine with the atoms to which they are attached to form an optionally substituted 3 to 8-membered heterocycloalkyl; or
R⁷ and R¹¹ combine with the atoms to which they are attached to form an optionally substituted 4 to 8-membered heterocycloalkyl. In some embodiments, X³ is N. In some embodiments, m is 1. In some embodiments, R¹¹ is hydrogen. In some embodiments, X³ is N, m is 1, and R¹¹ is H.

In some embodiments (e.g., of any one of Formulae DXa, DXa-1, DXa-2, DXa-3, DXa-4, or DXa-5), R⁹ is methyl.

In some embodiments of formula DIa or subformula thereof, a is 0. In some embodiments of formula DIa or subformula thereof, a is 0.

In some embodiments of formula DIa or subformula thereof, R² is optionally substituted C₁-C₆ alkyl. In some embodiments, R² is selected from —CH₂CH₃ or —CH₂CF₃.

In some embodiments of formula DIa or subformula thereof, W is C₁-C₄ alkyl. In some embodiments W is:

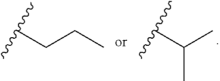

In some embodiments of formula DIa or subformula thereof, W is optionally substituted cyclopropyl, optionally substituted cyclobutyl, optionally substituted cyclopentyl, or optionally substituted cyclohexyl, optionally substituted piperidine, optionally substituted piperazine, optionally substituted pyridine, or optionally substituted phenyl.

In some embodiments of formula DIa or subformula thereof, W is optionally substituted 3 to 10-membered heterocycloalkyl, optionally substituted 3 to 10-membered cycloalkyl, optionally substituted 6 to 10-membered aryl, or optionally substituted 5 to 10-membered heteroaryl.

In some embodiments of formula DIa or subformula thereof, W is optionally substituted 3 to 10-membered heterocycloalkyl. In some embodiments, W is selected from the following, or a stereoisomer thereof:

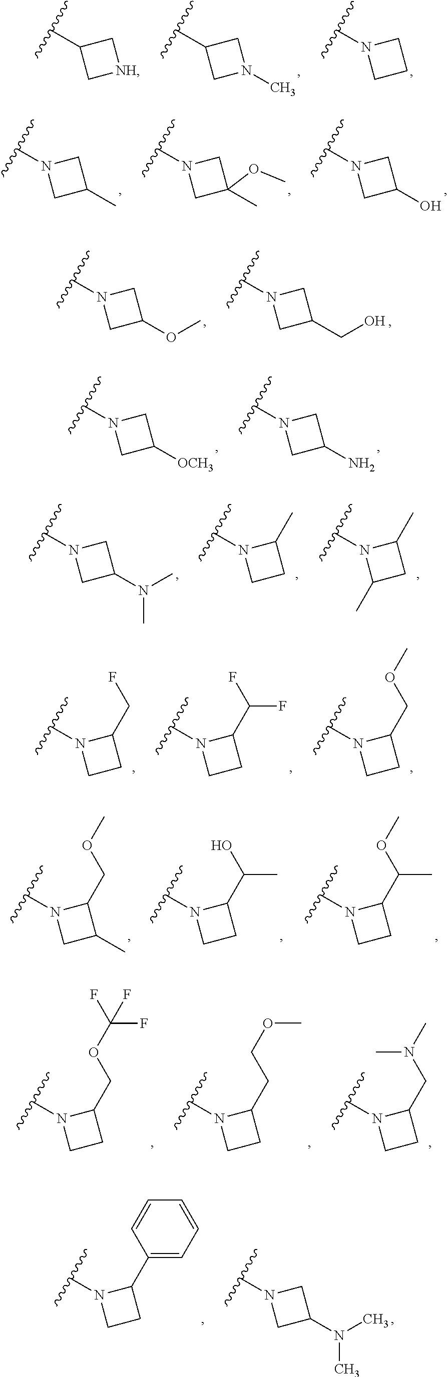

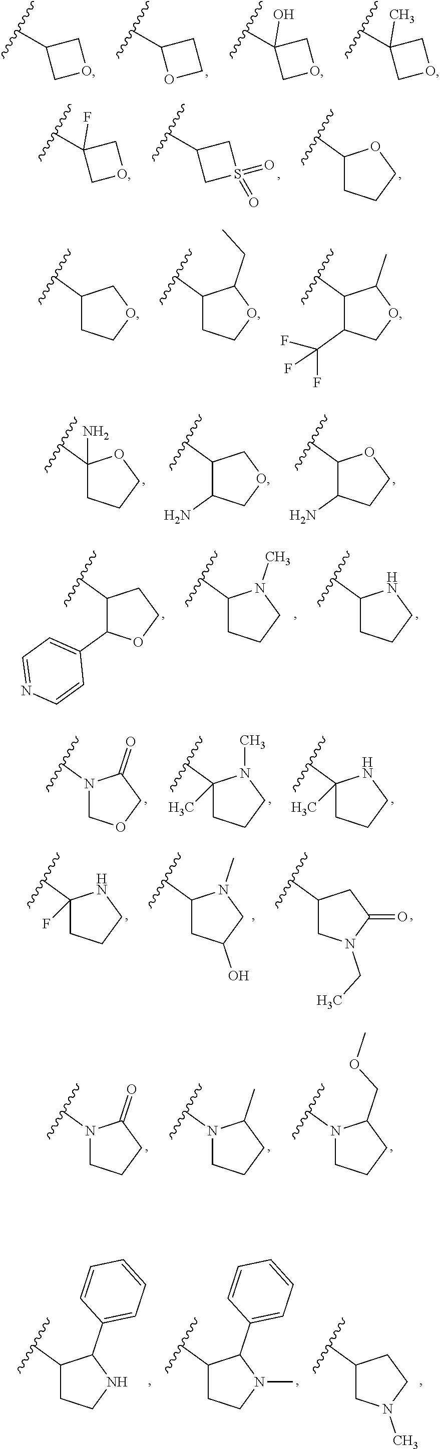

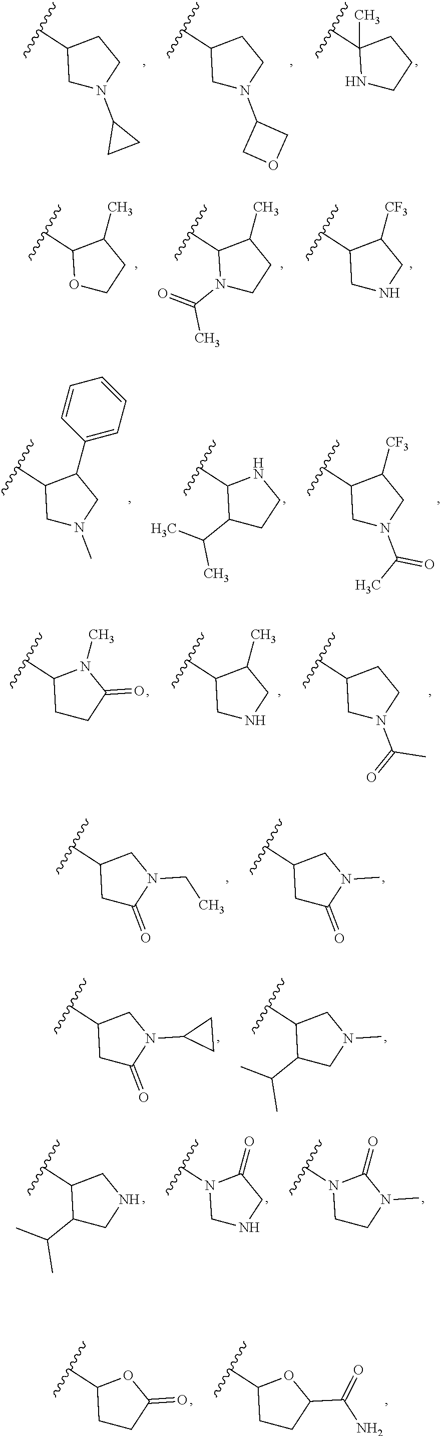

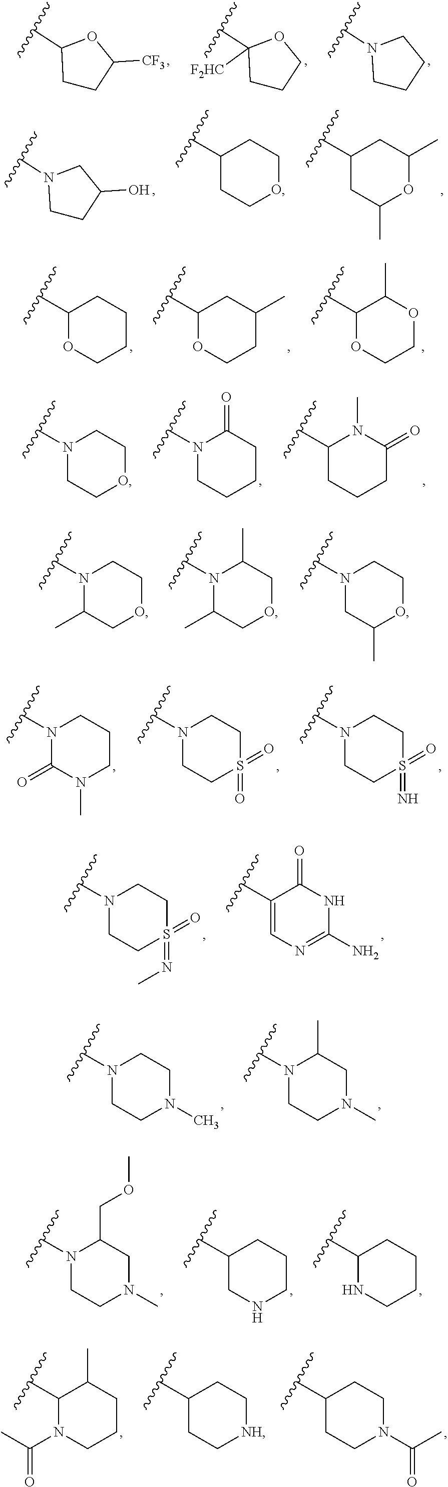

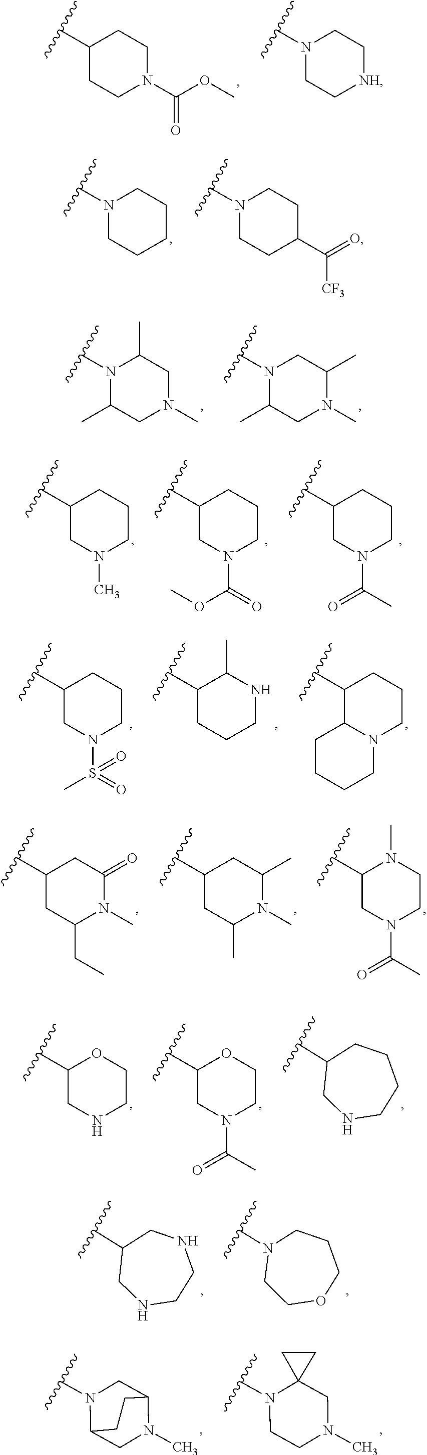

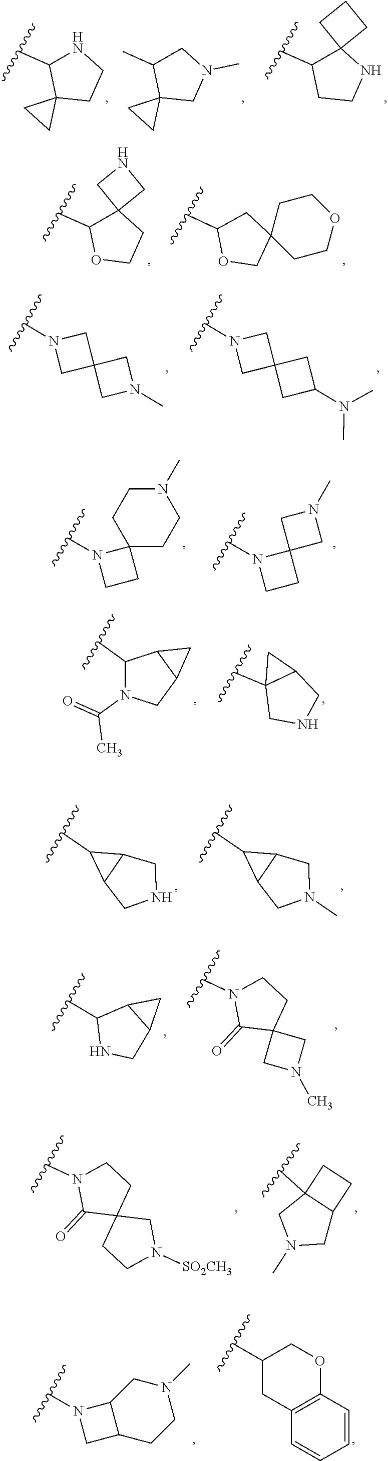

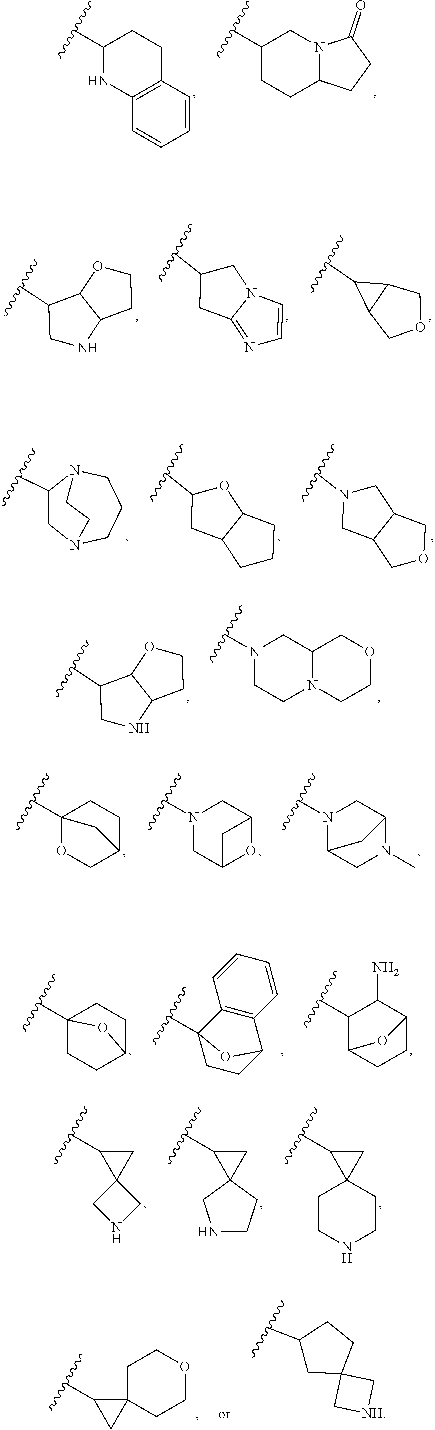

In some embodiments, W is selected from the following, or a stereoisomer thereof:

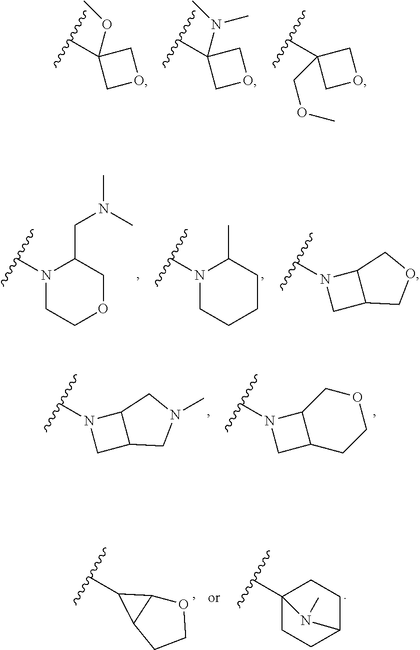

In some embodiments of formula DIa or subformula thereof, W is optionally substituted 3 to 10-membered cycloalkyl. In some embodiments, W is selected from the following, or a stereoisomer thereof:

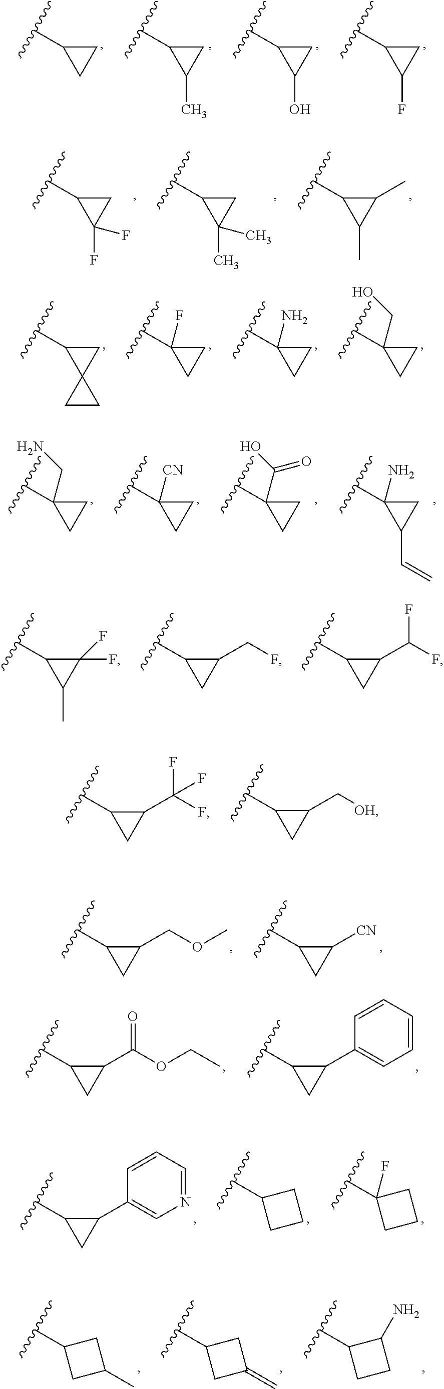

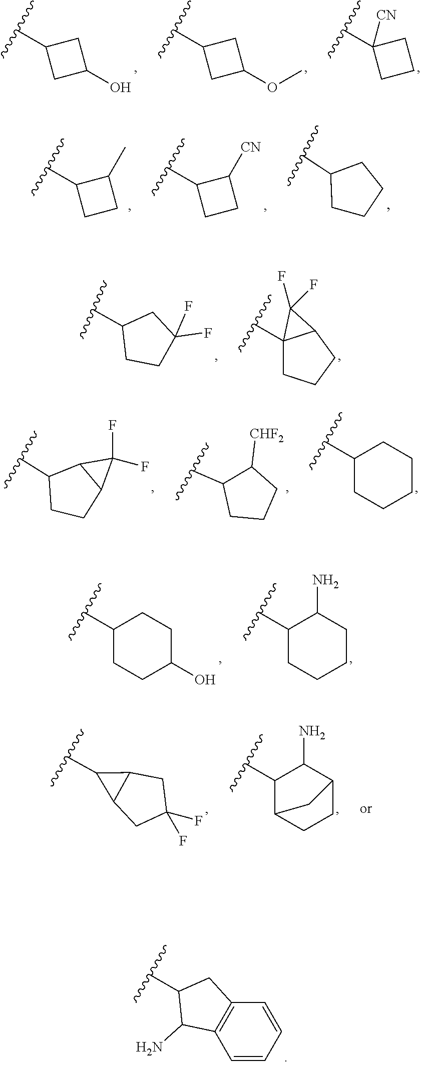

In some embodiments, W is selected from the following, or a stereoisomer thereof:

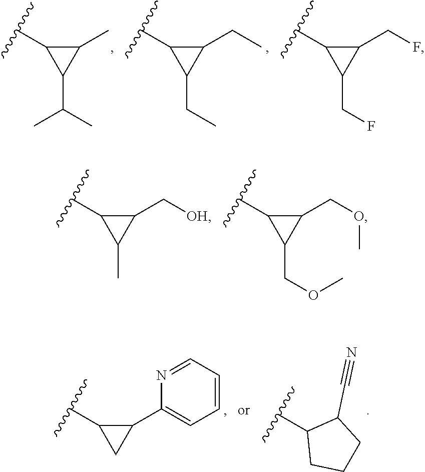

In some embodiments of formula DIa or subformula thereof, W is optionally substituted 5 to 10-membered heteroaryl. In some embodiments, W is selected from the following, or a stereoisomer thereof:

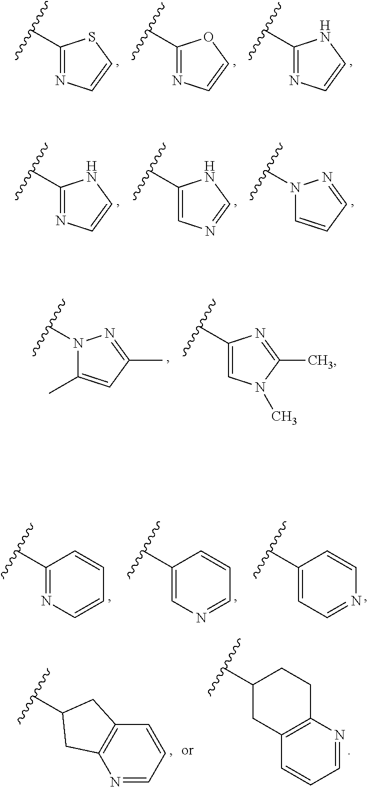

In some embodiments of formula DIa or subformula thereof, W is optionally substituted 6 to 10-membered aryl. In some embodiments, W is optionally substituted phenyl.

In some embodiments of formula DIa or subformula thereof, W is optionally substituted C₁-C₃ heteroalkyl. In some embodiments, W is selected from the following, or a stereoisomer thereof:

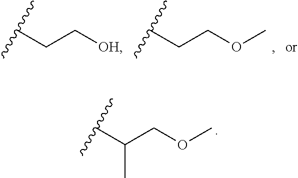

In some embodiments, the RAS(ON) inhibitor, or pharmaceutically acceptable salt thereof, has the structure of Formula DIb:

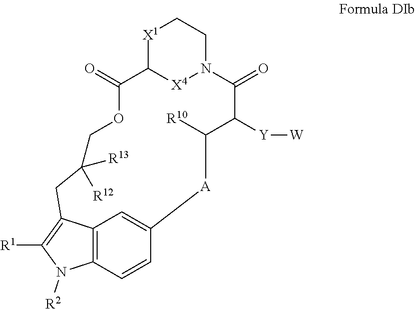

wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;

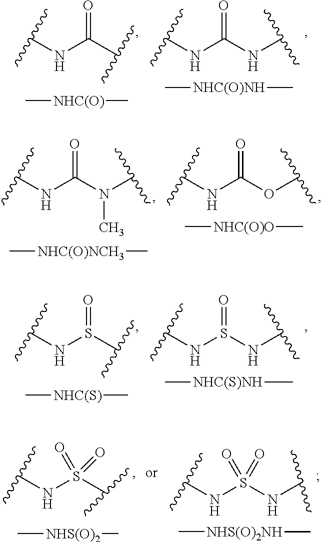

W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl; or W is —R¹⁴C(═O)R¹⁵ where R¹⁴ is 3 to 10-membered cycloalkylene and R¹⁵ is selected from optionally substituted 3 to 10-membered cycloalkyl, optionally substituted 6 to 10-membered aryl, or optionally substituted 5 to 10-membered heteroaryl;
X¹ and X⁴ are each, independently, CH₂, CH(CH₃) or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl;
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R¹⁰ is hydrogen, hydroxy, optionally substituted C₁-C₆ alkoxy, optionally substituted C₁-C₃ alkyl, optionally substituted C₁-C₆ heteroalkyl, or optionally substituted 3 to 7-membered heterocycloalkyl; and
R¹² and R¹³ are each, independently, selected from F or CH₃, or R¹² and R¹³ combine with the atoms to which they are attached to make a 3-membered cycloalkyl

In some embodiments, the RAS(ON) inhibitor is selected from Table D1a, or a pharmaceutically acceptable salt or stereoisomer thereof. In some embodiments, the RAS(ON) inhibitor is selected from Table D1a, or a pharmaceutically acceptable salt or atropisomer thereof.

TABLE D1a
Certain Compounds of the Present Invention

| Ex. | | Ex. | |
|---|---|---|---|
| # | Structure | # | Structure |
| DA 1 | 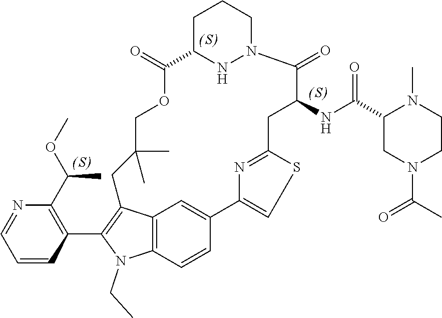 | DA 177 | 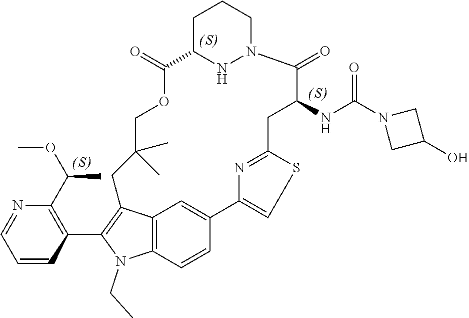 |
| DA 2 | 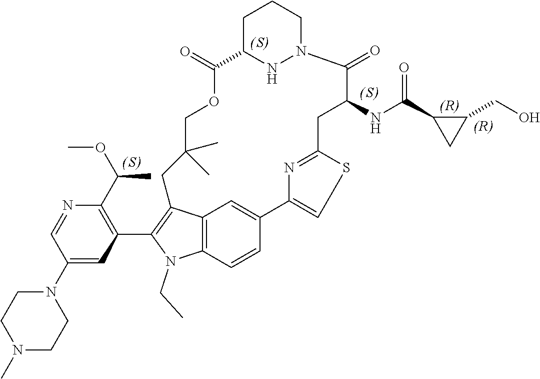 | DA 178 | 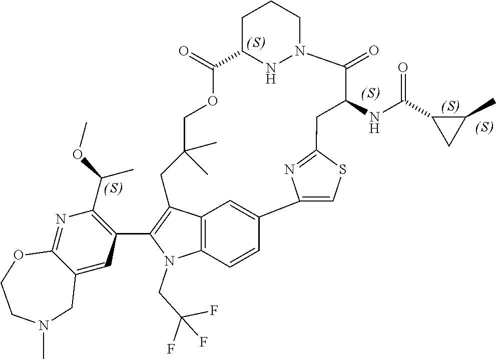 |
| DA 3 | 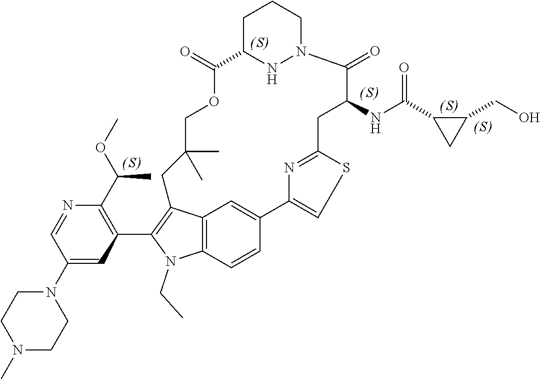 | DA 179 | 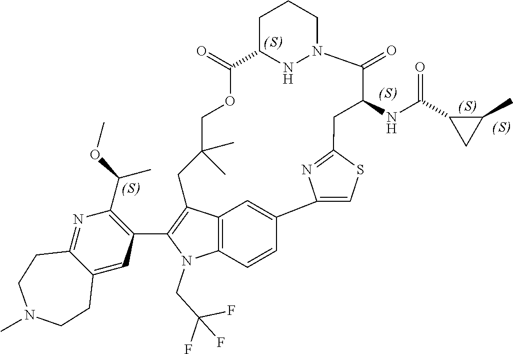 |
| DA 4 | 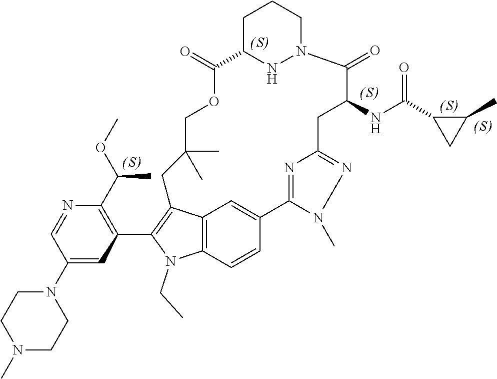 | DA 180 | 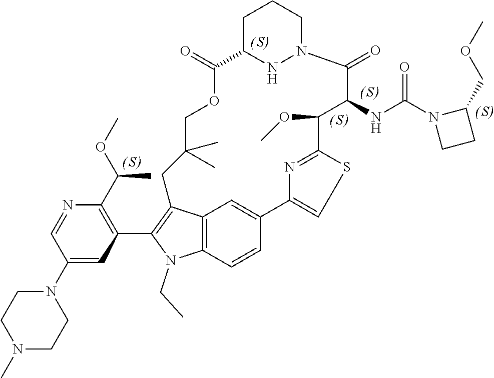 |
| DA 5 | 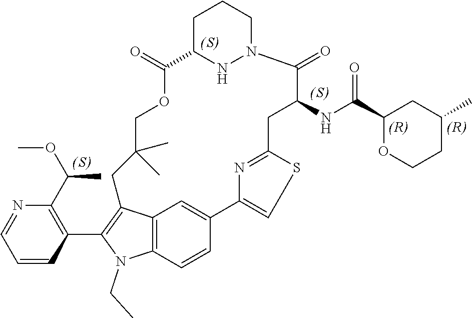 | DA 181 | 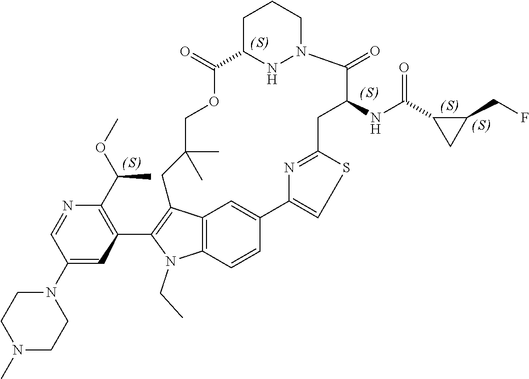 |
| DA 6 | 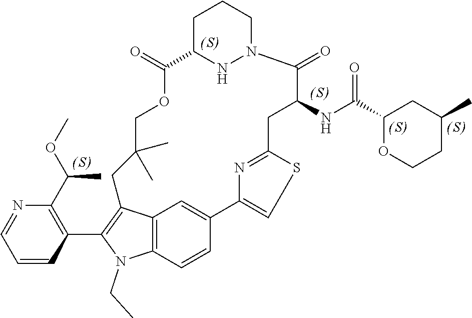 | DA 182 | 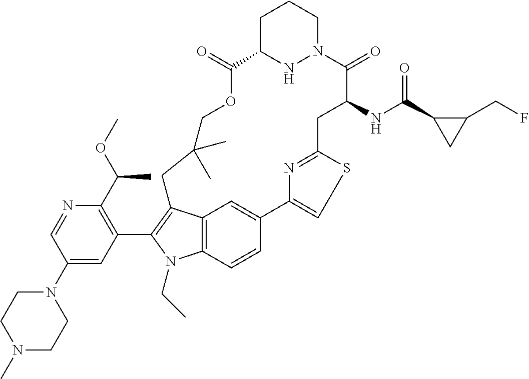 [SMILES: CCn1c(-c2cc(N3CCN(C)CC3)cnc2[C@H](C)OC)c2c3cc(ccc31)-c1csc(n1)C[C@H](NC(=O)[C@@H]1CC1CF)C(=O)N1CCC[C@H](N1)C(=O)OCC(C)(C)C2] |
| DA 7 | 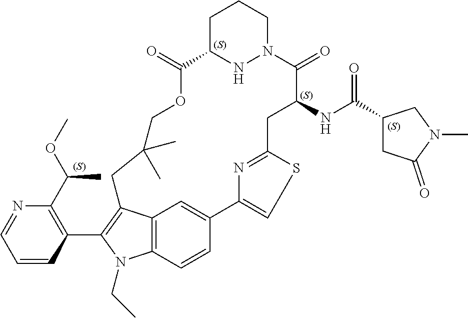 | DA 183 | 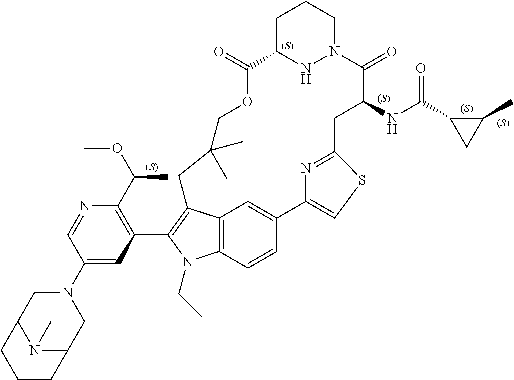 |
| DA 8 | 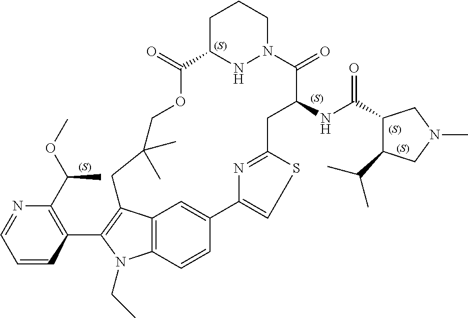 | DA 184 | 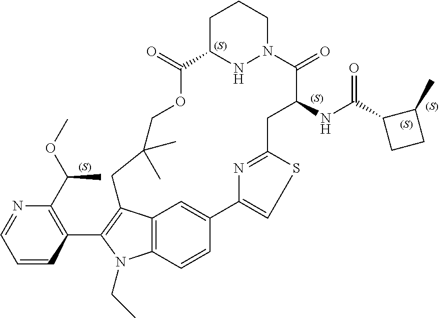 |
| DA 9 | 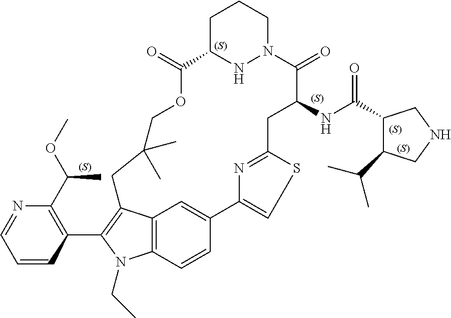 | DA 185 | 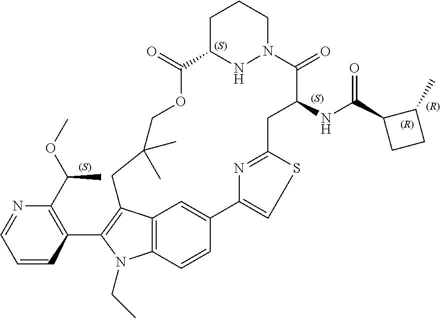 |
| DA 10 | 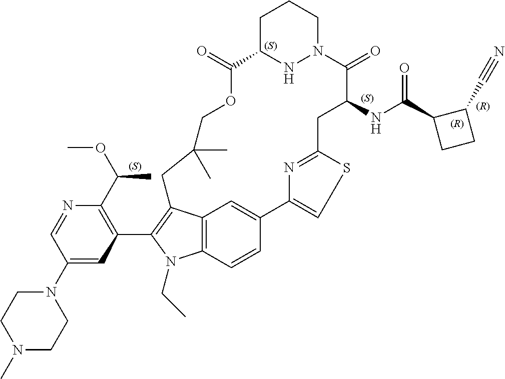 | DA 186 | 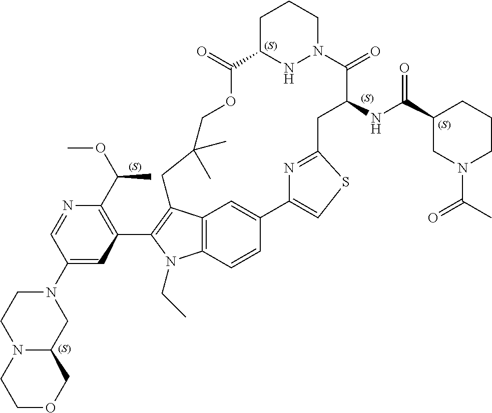 |
| DA 11 | 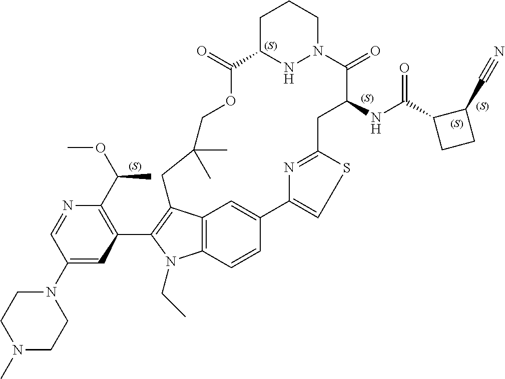 | DA 187 | 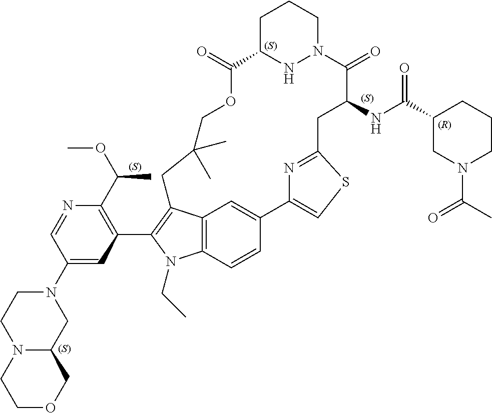 |
| DA 12 | 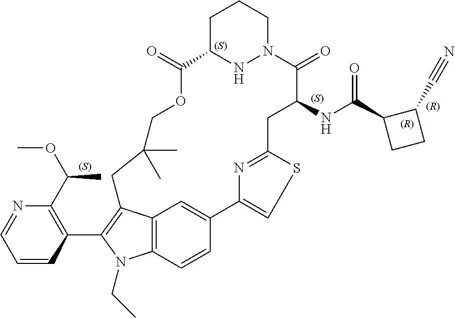 | DA 188 | 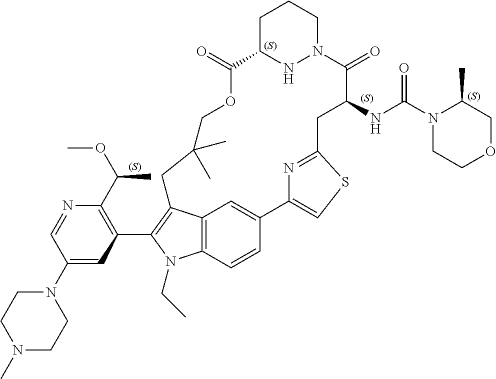 |
| DA 13 | 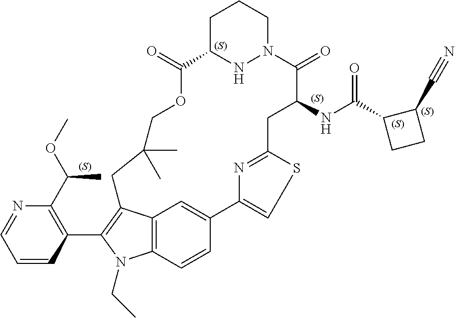 | DA 189 | 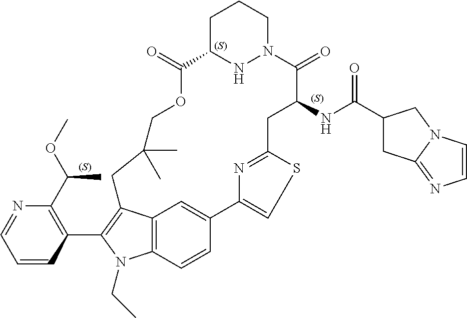 |
| DA 14 | 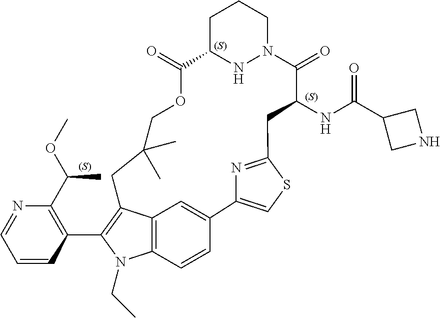 | DA 190 | 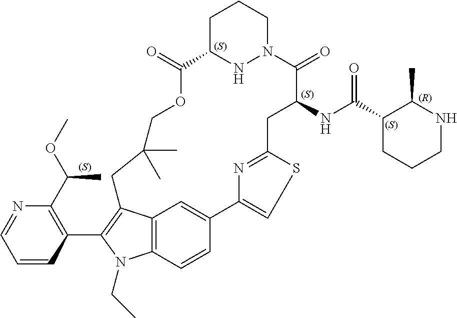 |
| DA 15 | 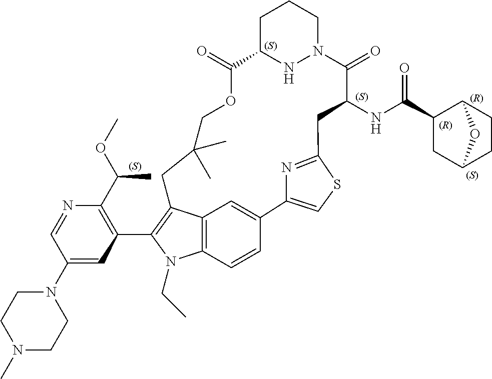 | DA 191 | 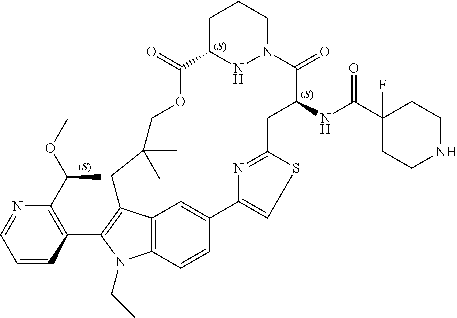 |
| DA 16 | 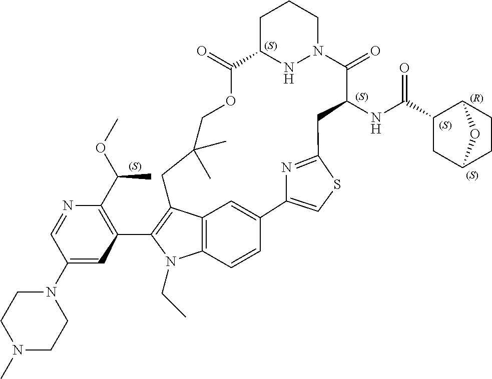 | DA 192 | 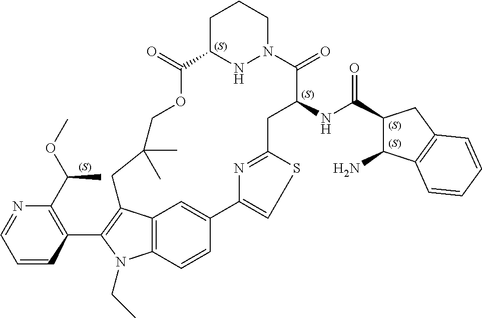 |
| DA 17 | 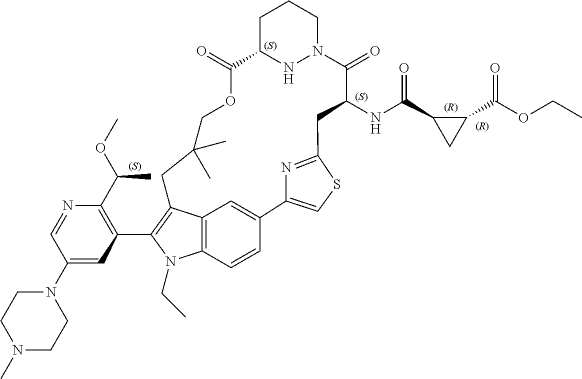 | DA 193 | 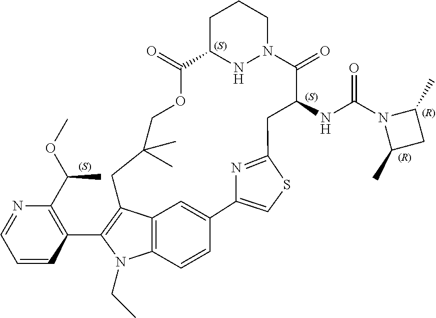 |
| DA 18 | 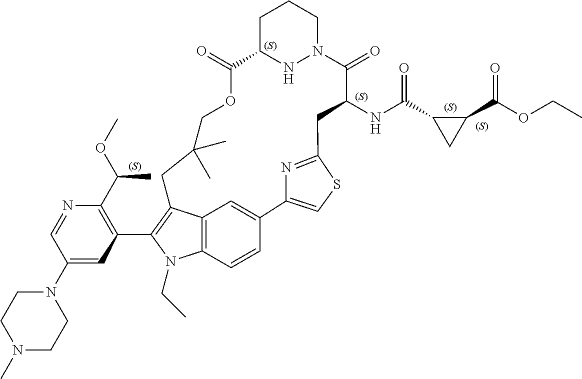 | DA 194 | 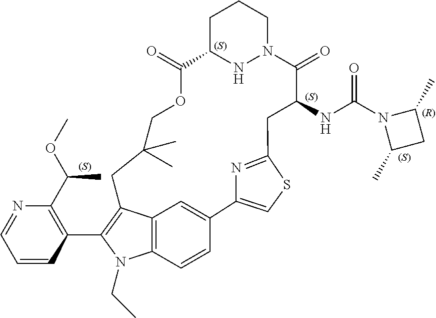 |
| DA 19 | 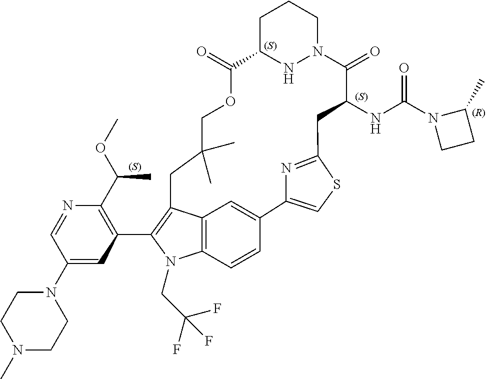 | DA 195 | 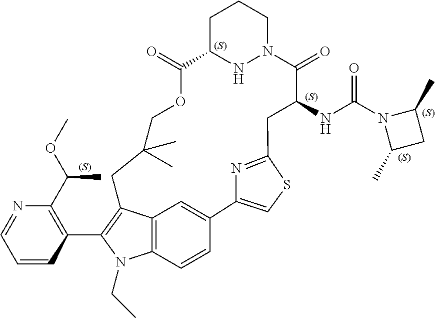 |
| DA 20 | 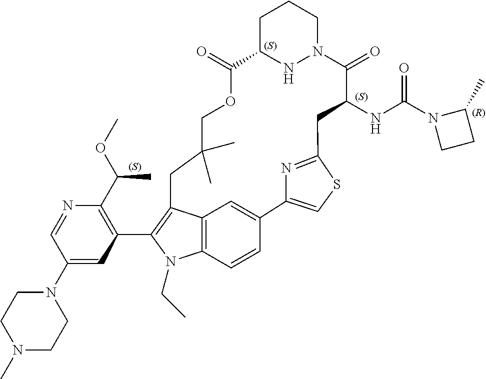 | DA 196 | 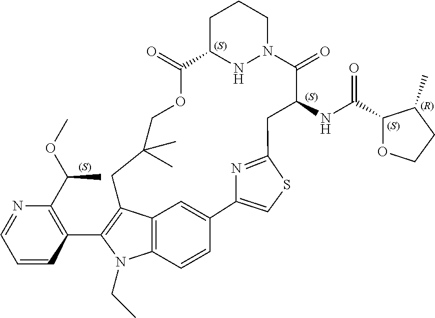 |
| DA 21 | 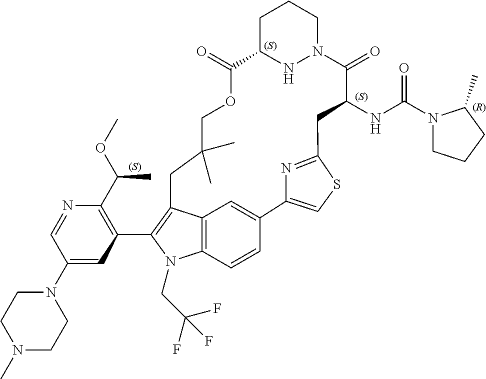 | DA 197 | 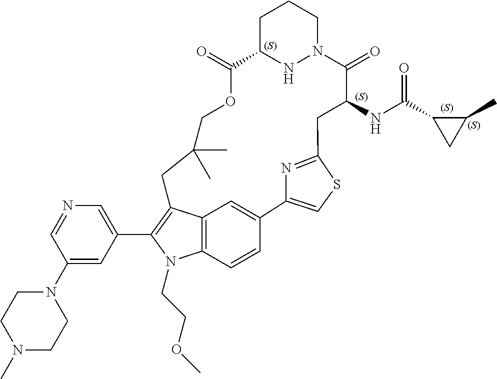 |
| DA 22 | 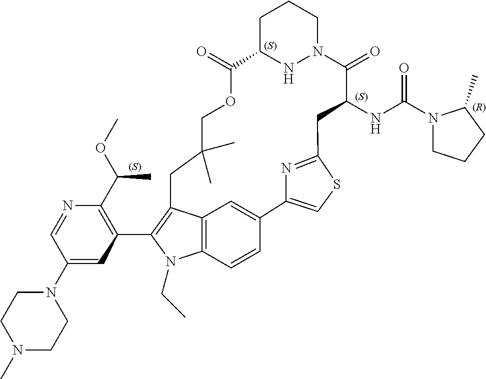 | DA 198 | 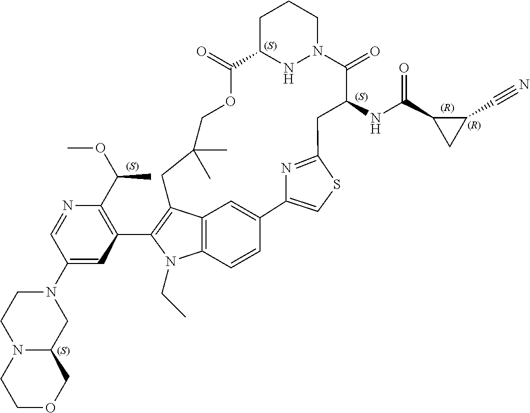 |
| DA 23 | 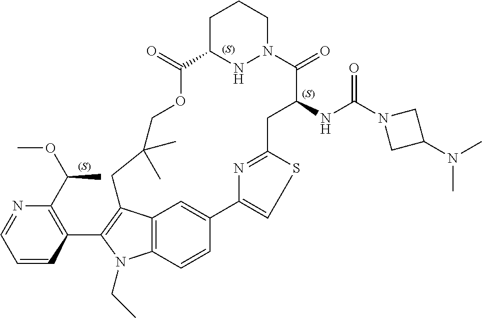 | DA 199 | 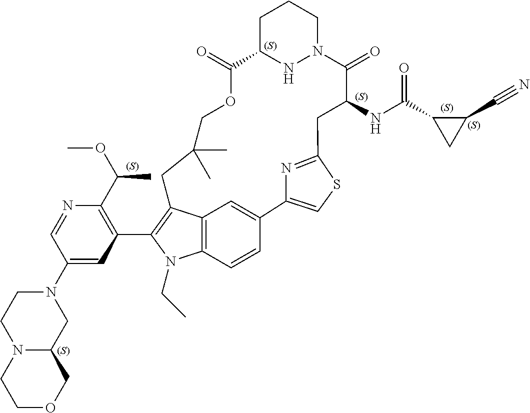 |
| DA 24 | 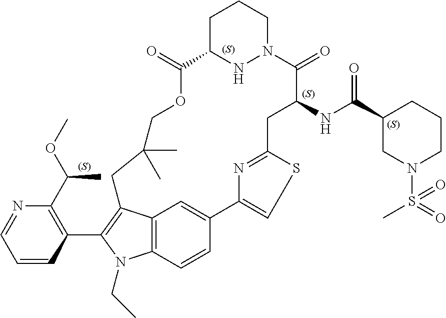 | DA 200 | 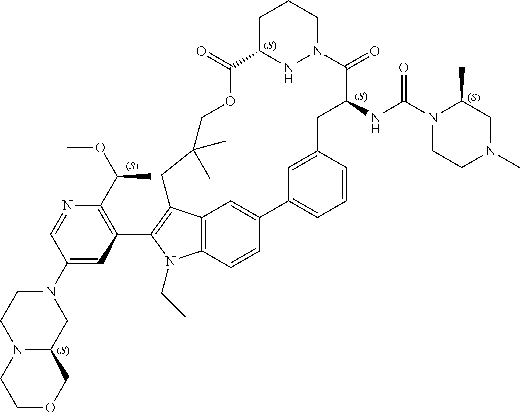 |
| DA 25 | 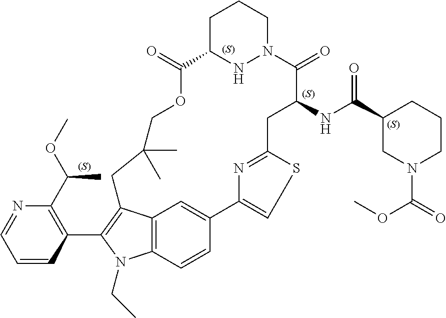 | DA 201 | 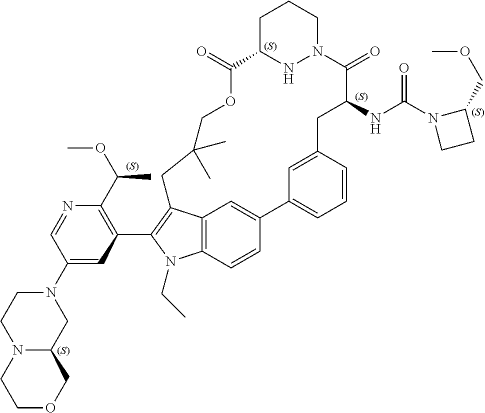 |
| DA 26 | 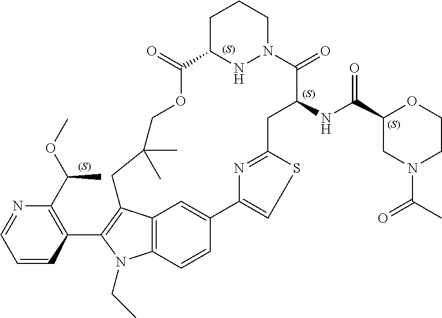 | DA 202 | 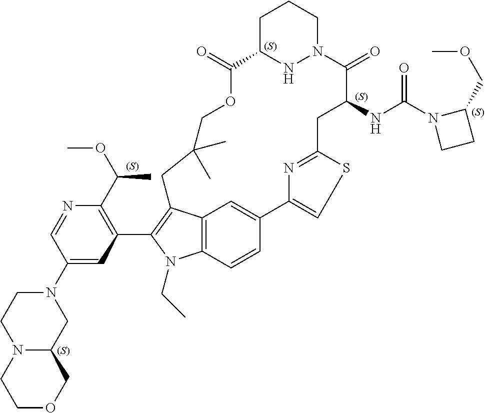 |
| DA 27 | 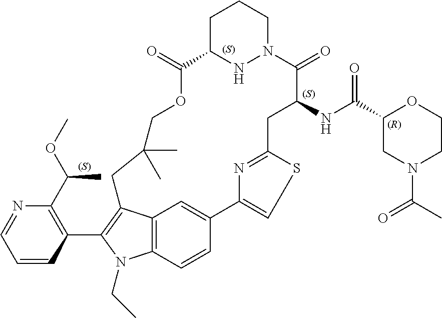 | DA 203 | 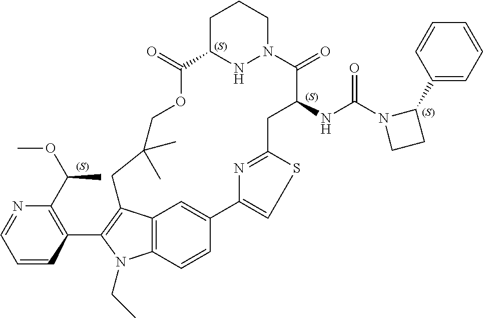 |
| DA 28 | 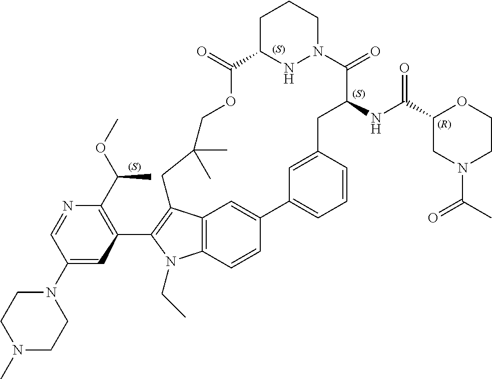 | DA 204 | 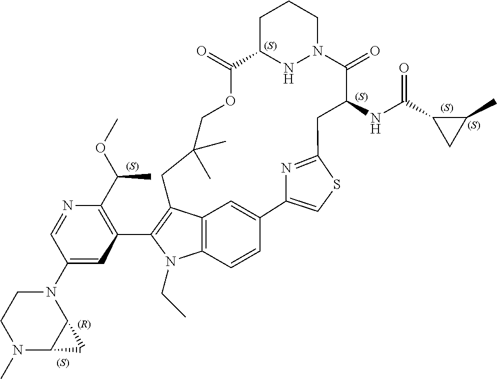 |
| DA 29 | 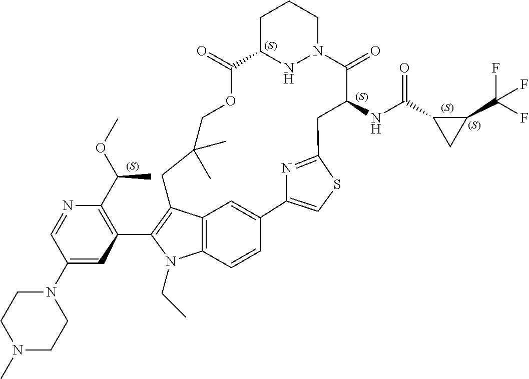 | DA 205 | 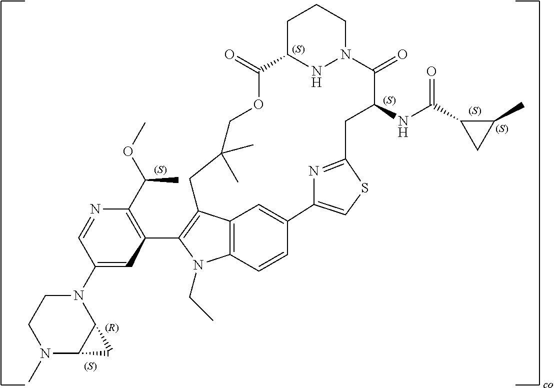 |
| DA 30 | 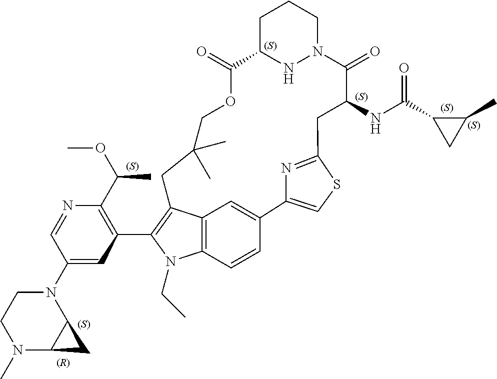 | DA 206 | 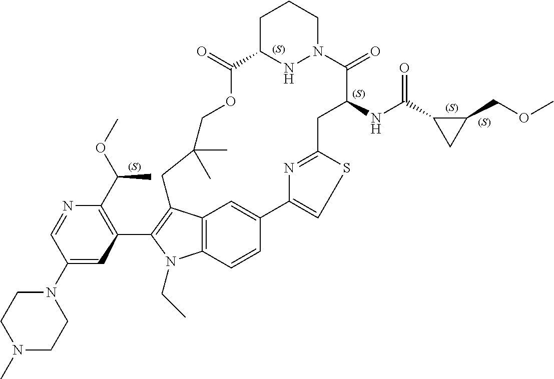 |
| DA 31 | 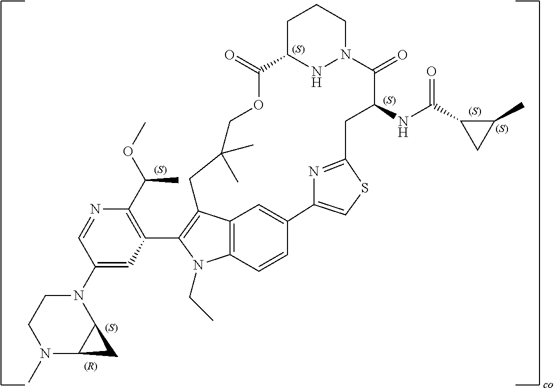 | DA 207 | 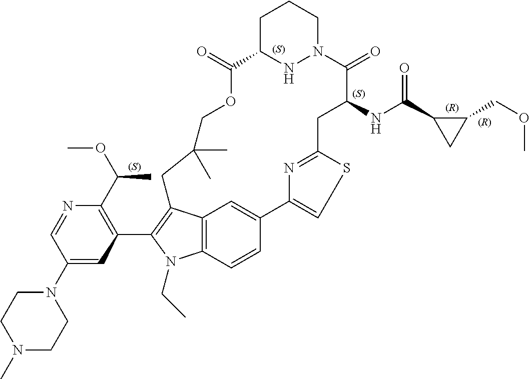 |
| DA 32 | 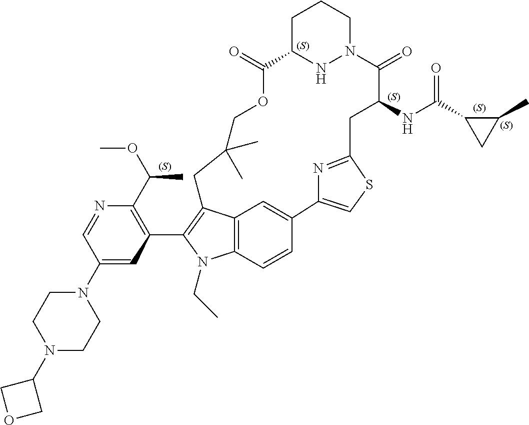 | DA 208 | 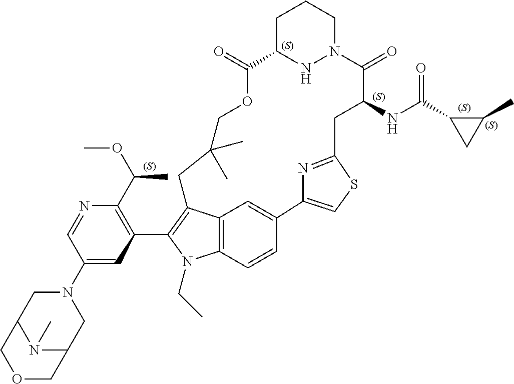 |
| DA 33 | 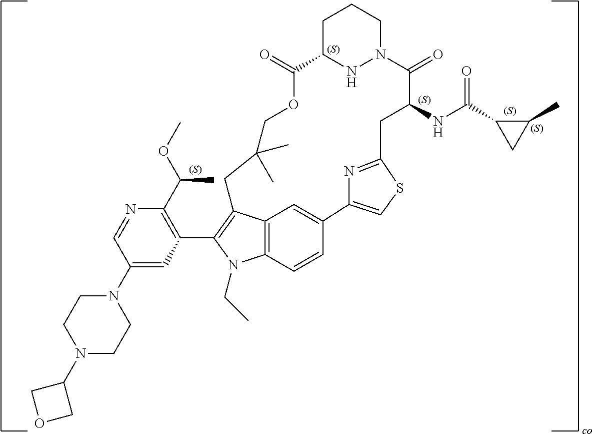 | DA 209 | 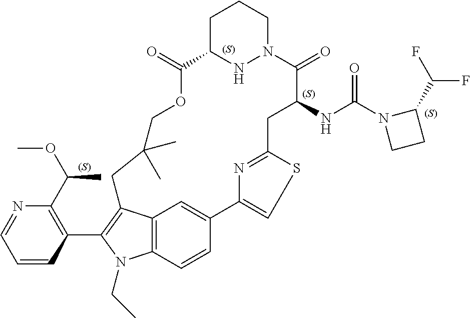 |
| DA 34 | 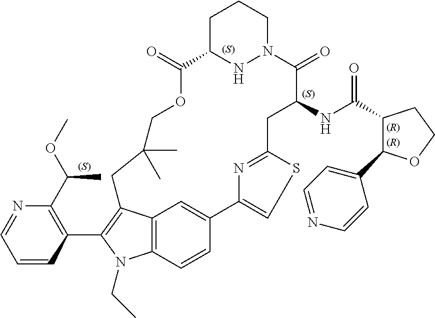 | DA 210 | 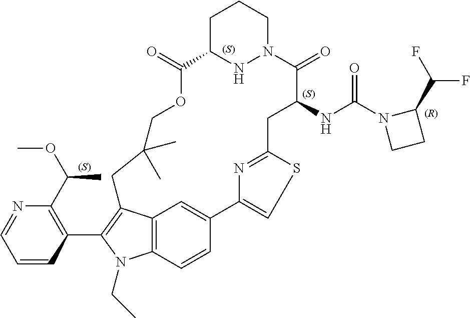 |
| DA 35 | 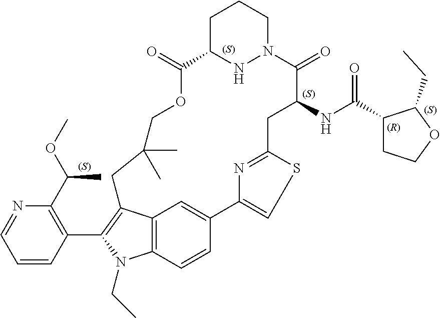 | DA 211 | 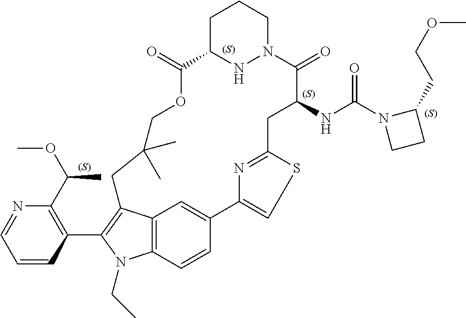 |
| DA 36 | 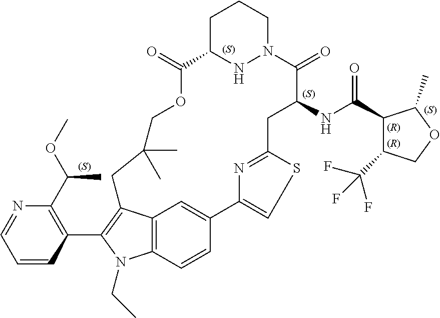 | DA 212 | 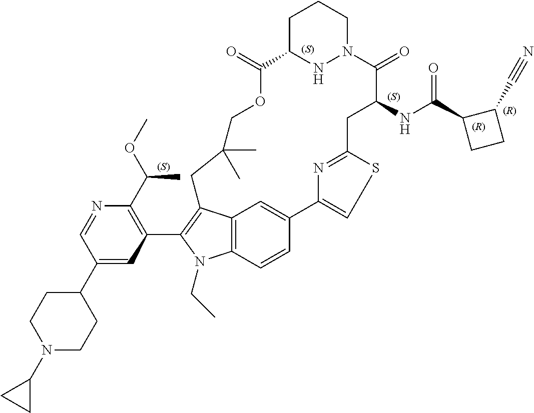 |
| DA 37 | 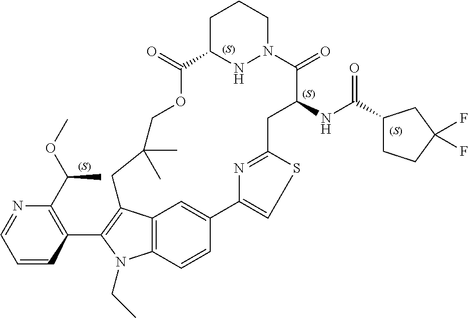 | DA 213 | 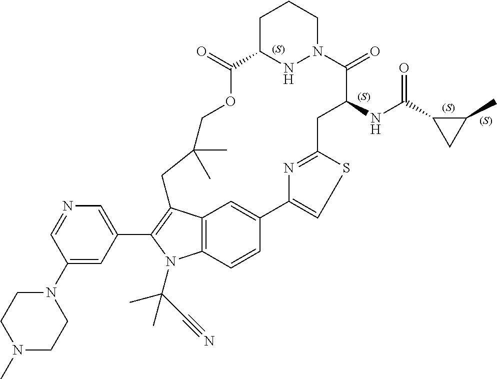 |
| DA 38 | 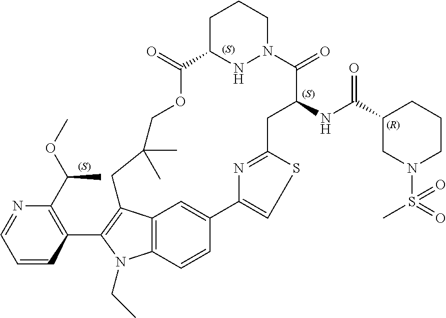 | DA 214 | 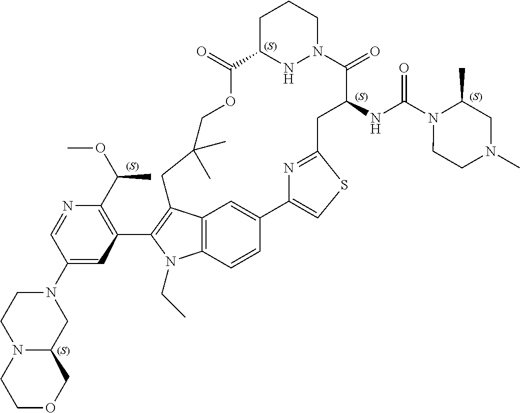 |
| DA 39 | 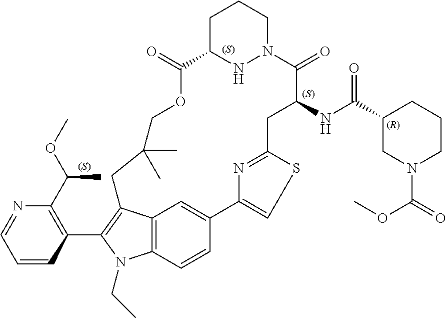 | DA 215 | 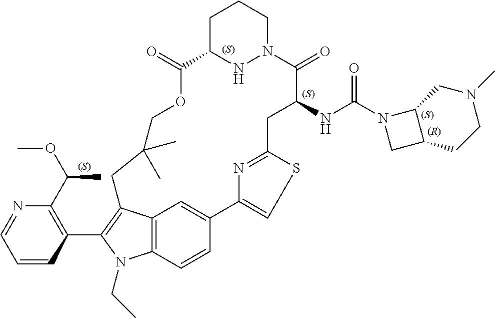 |
| DA 40 | 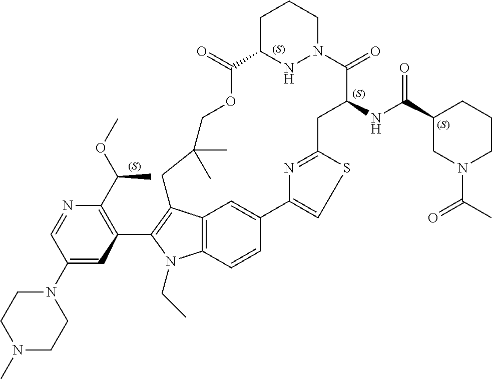 | DA 216 | 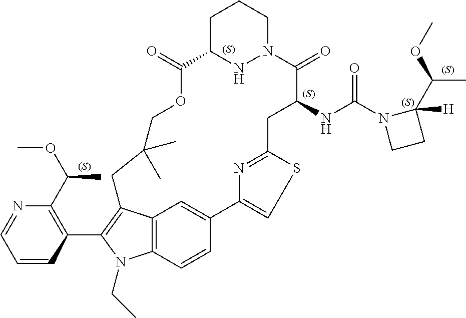 |
| DA 41 | 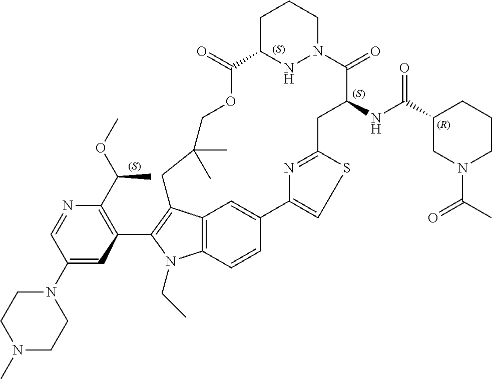 | DA 217 | 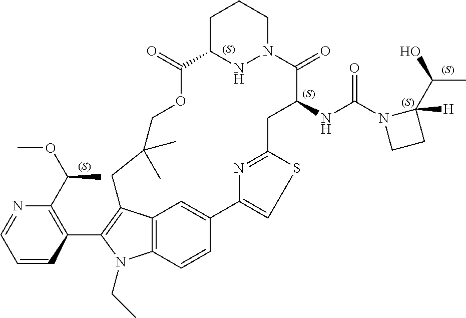 |
| DA 42 | 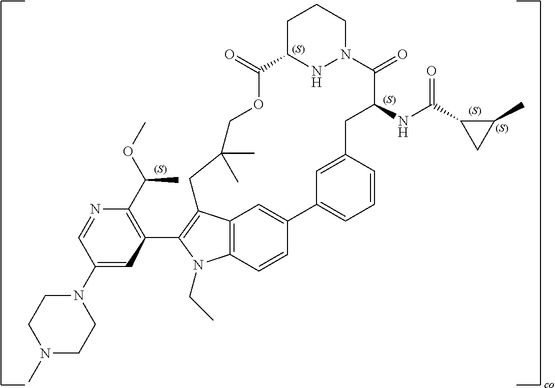 | DA 218 | 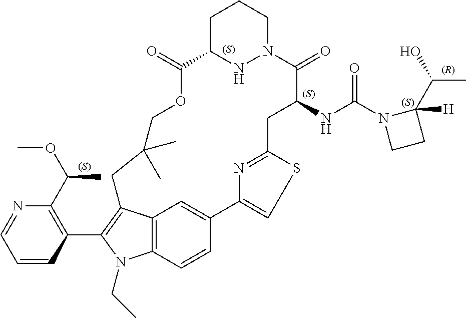 |
| DA 43 | 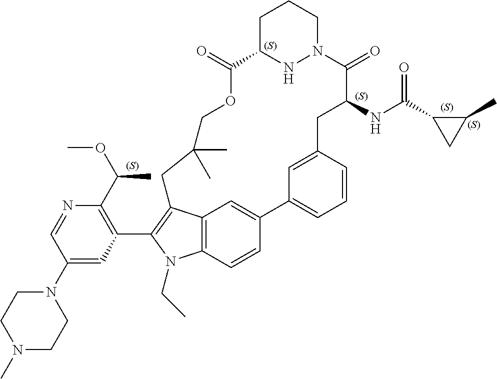 | DA 219 | 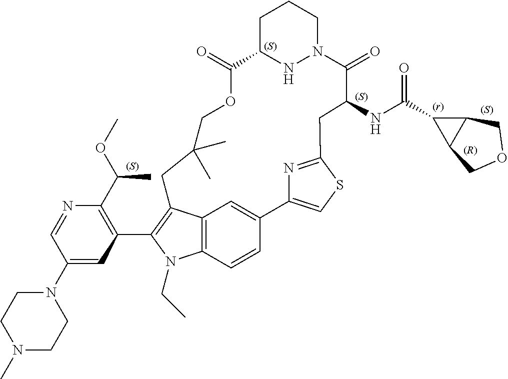 |
| DA 44 | 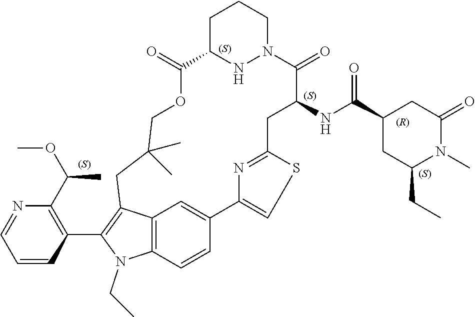 | DA 220 | 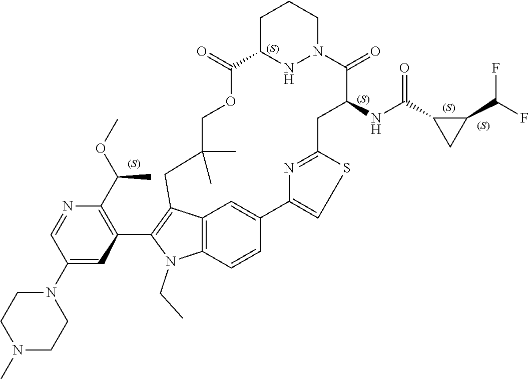 |
| DA 45 | 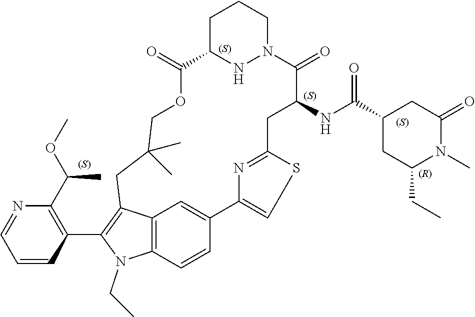 | DA 221 | 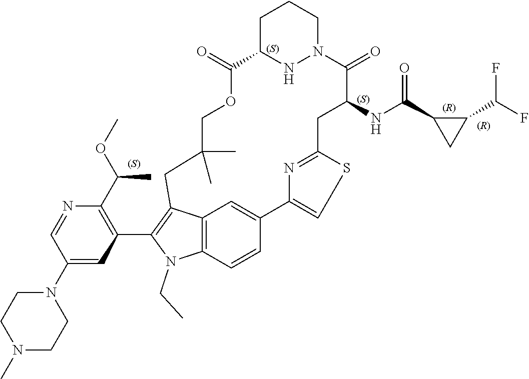 |
| DA 46 | 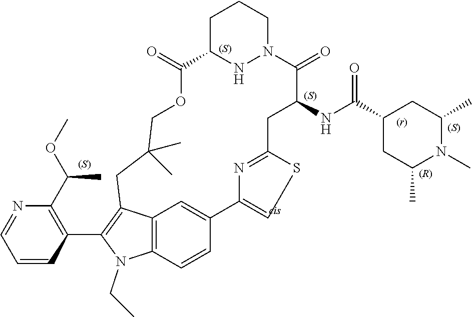 | DA 222 | 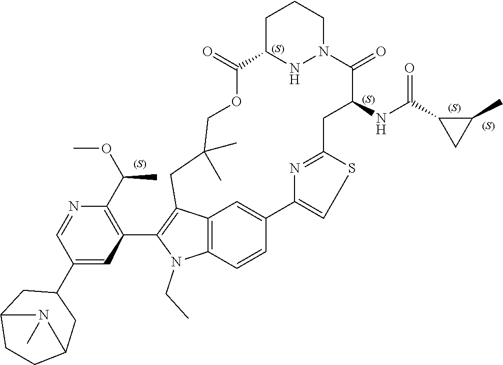 |
| DA 47 | 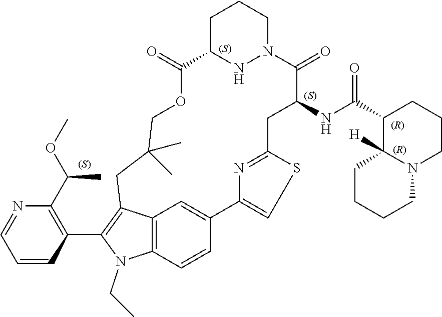 | DA 223 | 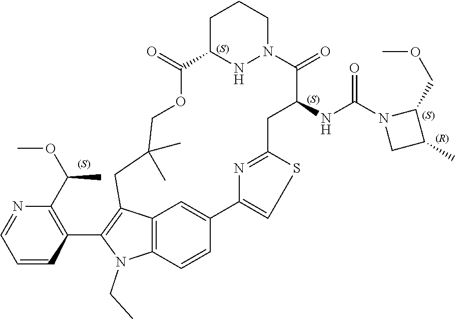 |
| DA 48 | 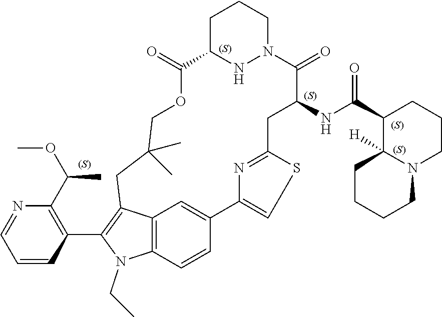 | DA 224 | 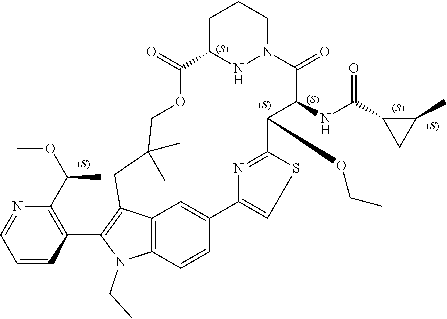 |
| DA 49 | 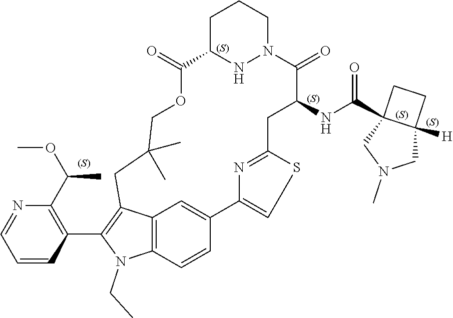 | DA 225 | 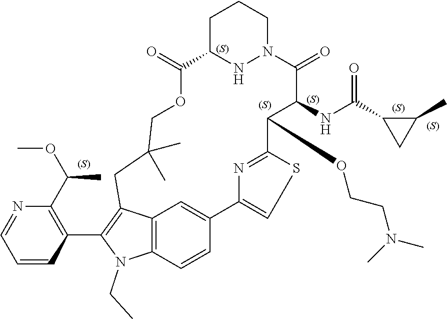 |
| DA 50 | 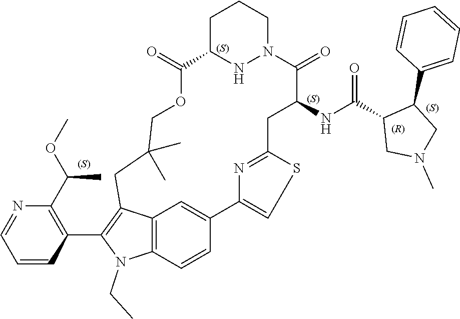 | DA 226 | 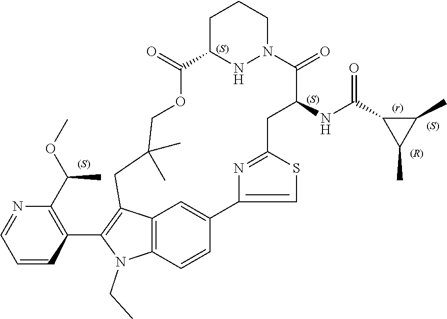 |
| DA 51 | 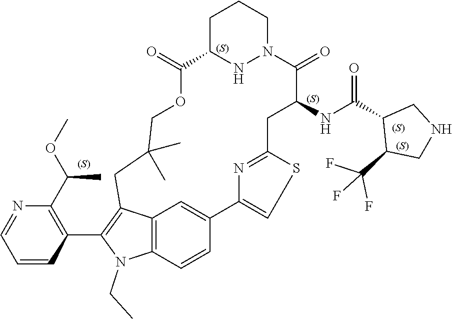 | DA 227 | 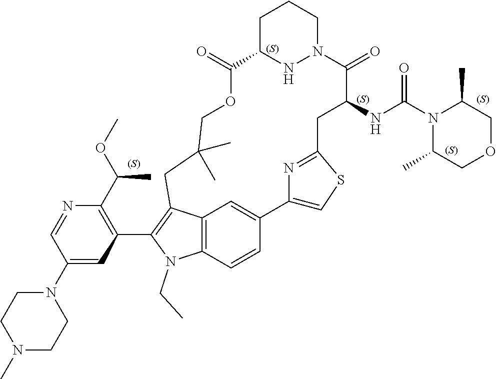 |
| DA 52 | 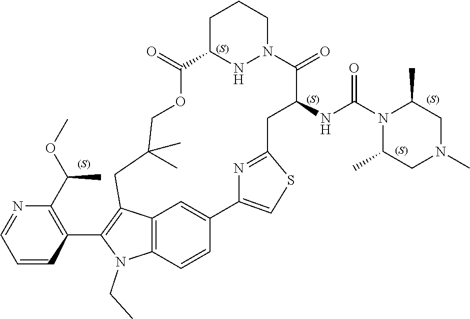 | DA 228 | 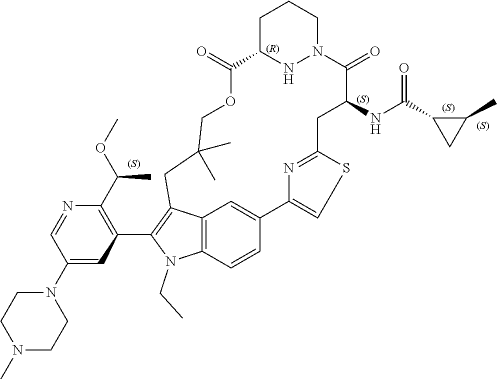 |
| DA 53 | 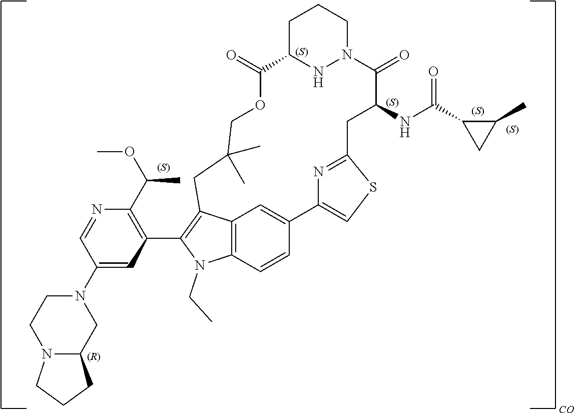 | DA 229 | 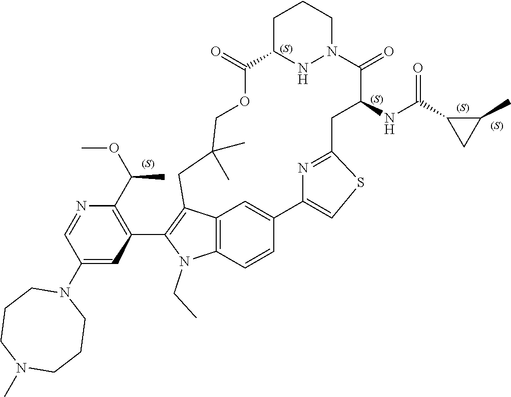 |
| DA 54 | 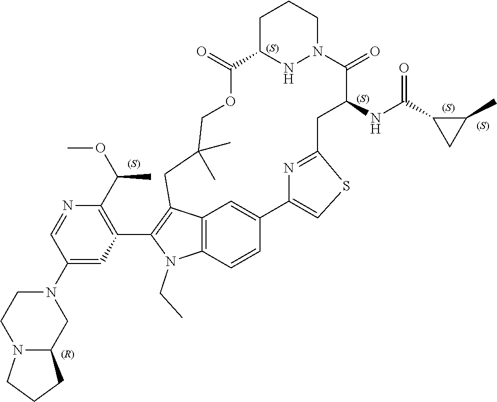 | DA 230 | 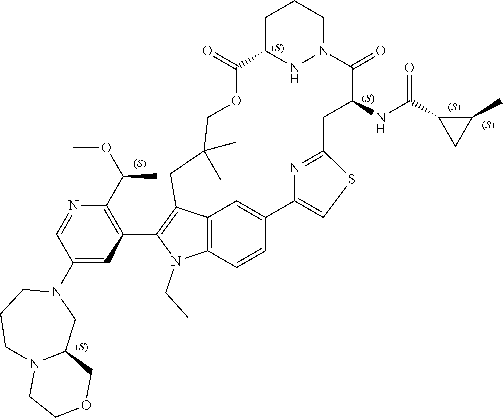 |
| DA 55 | 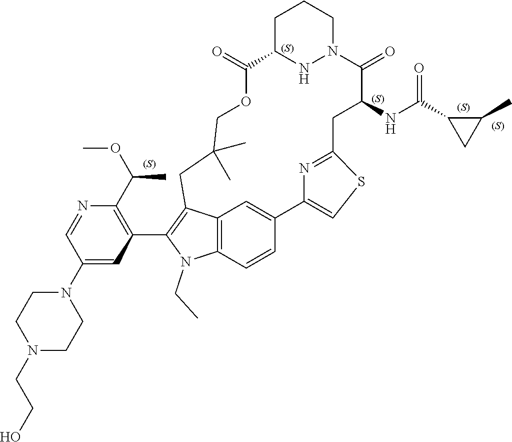 | DA 231 | 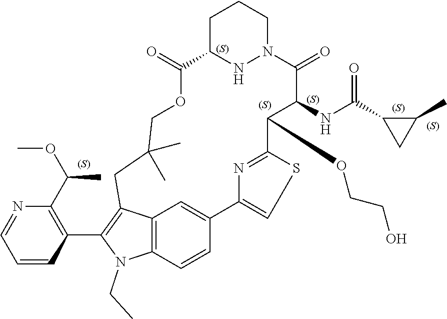 |
| DA 56 | 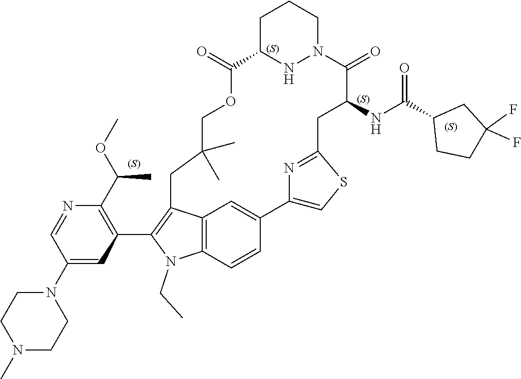 | DA 232 | 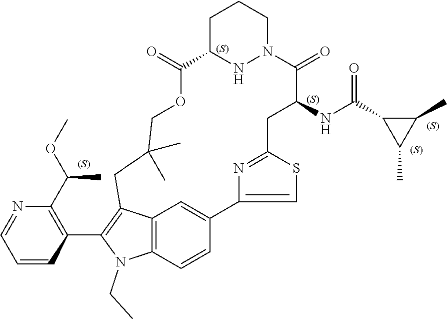 |
| DA 57 | 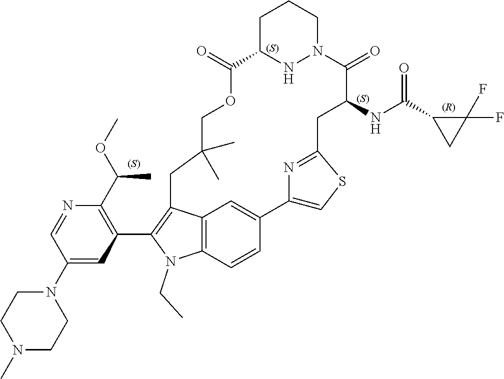 | DA 233 | 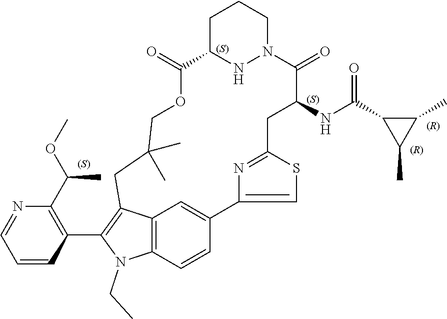 |
| DA 58 | 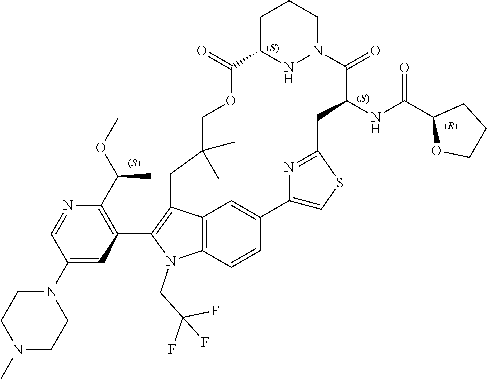 | DA 234 | 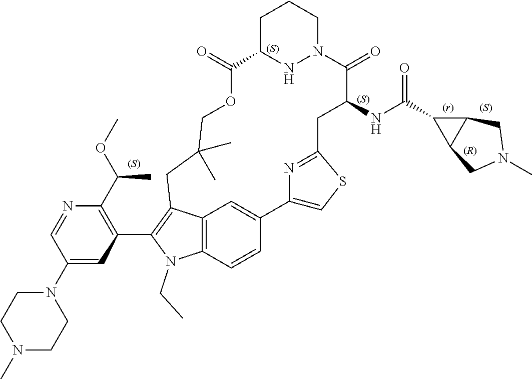 |
| DA 59 | 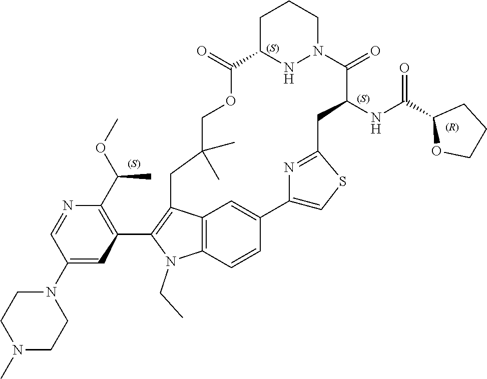 | DA 235 | 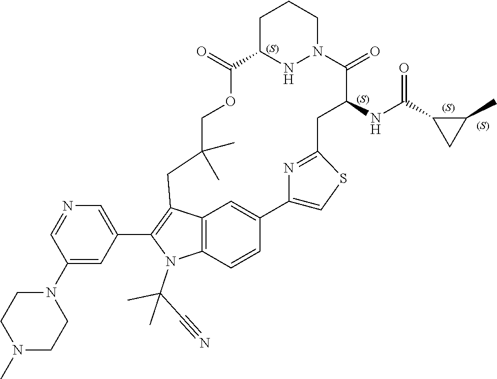 |
| DA 60 | 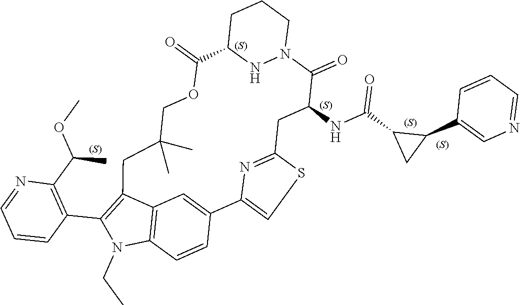 | DA 236 | 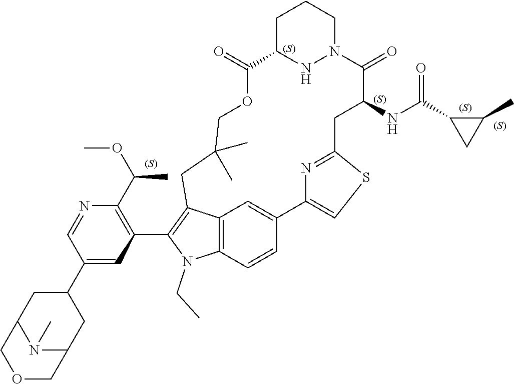 |
| DA 61 | 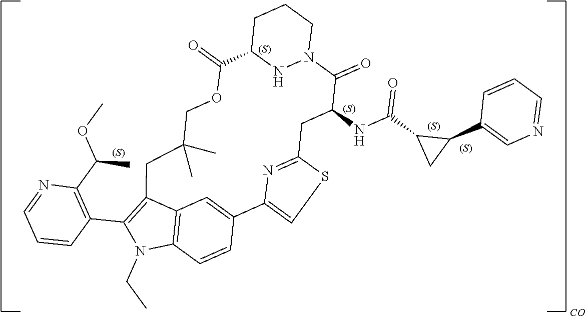 | DA 237 | 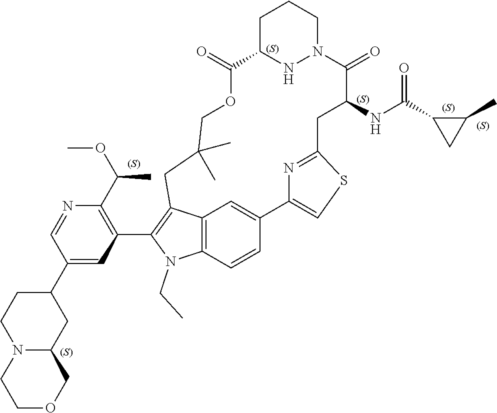 |
| DA 62 | 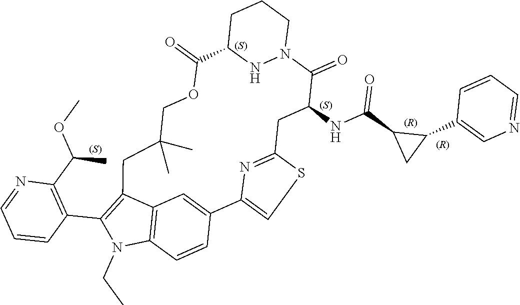 | DA 238 | 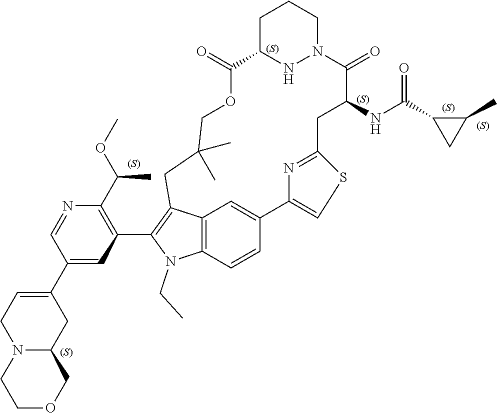 |
| DA 63 | 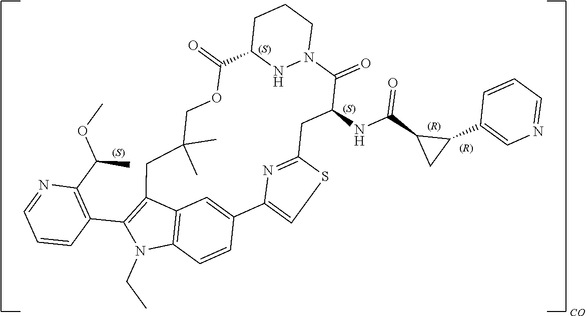 | DA 239 | 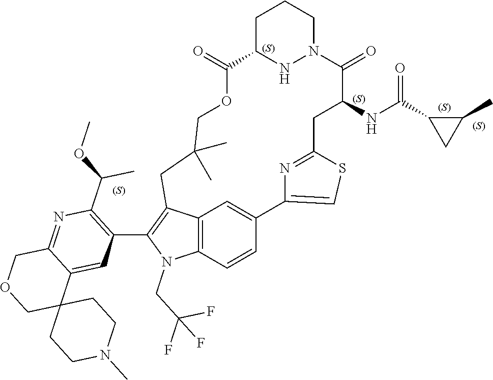 |
| DA 64 | 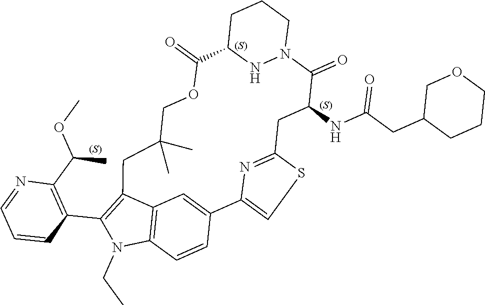 | DA 240 | 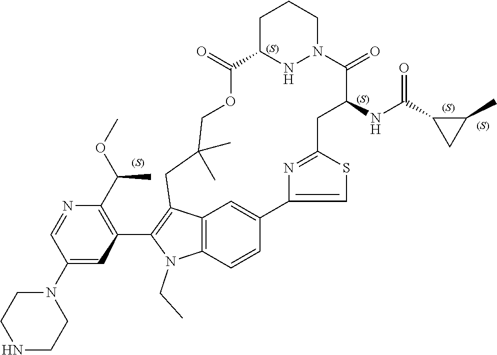 |
| DA 65 | 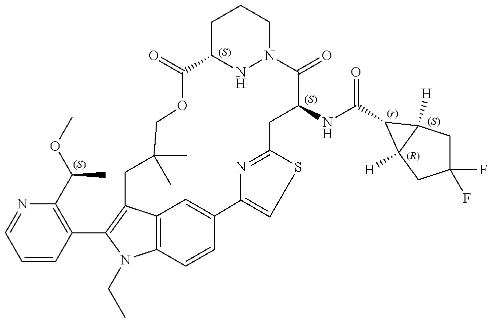 | DA 241 | 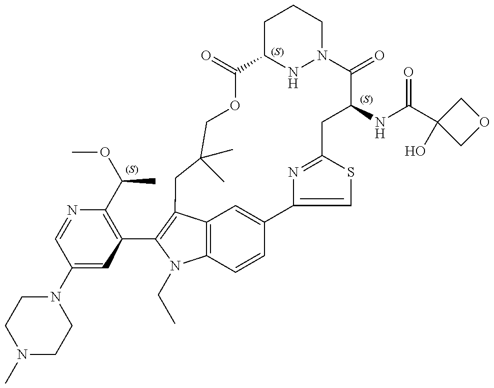 |
| DA 66 | 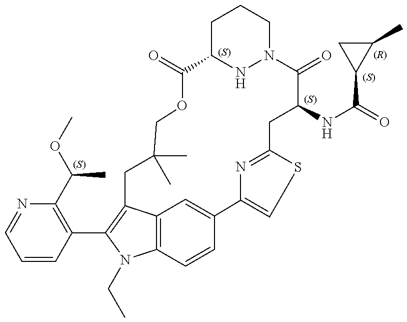 | DA 242 | 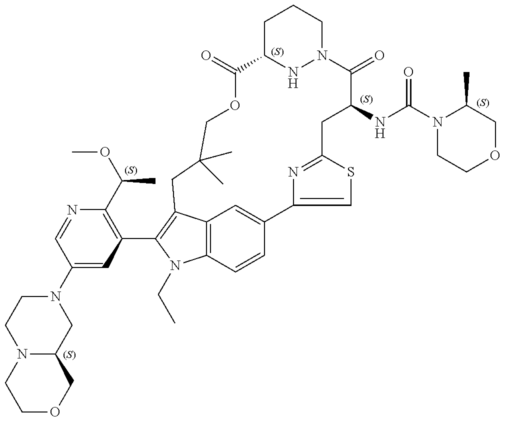 |
| DA 67 | 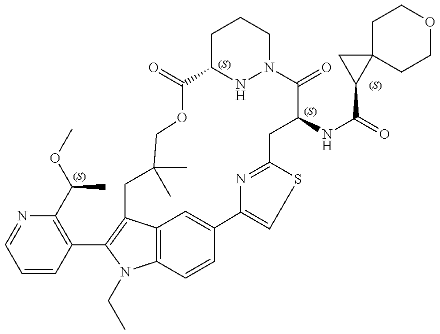 | DA 243 | 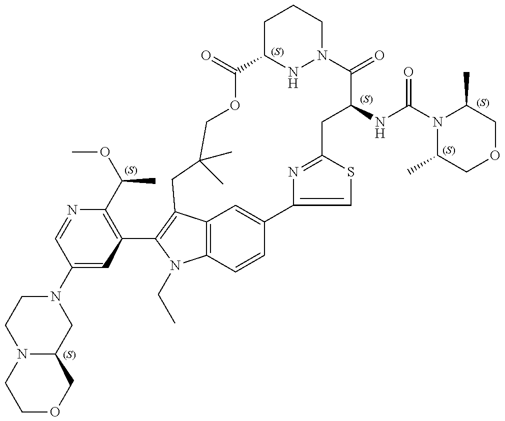 |
| DA 68 | 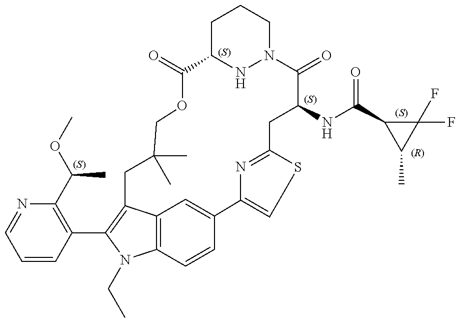 | DA 244 | 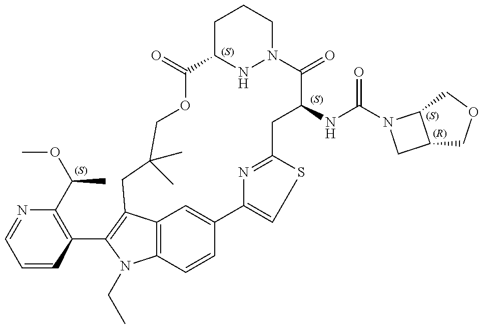 |
| DA 69 | 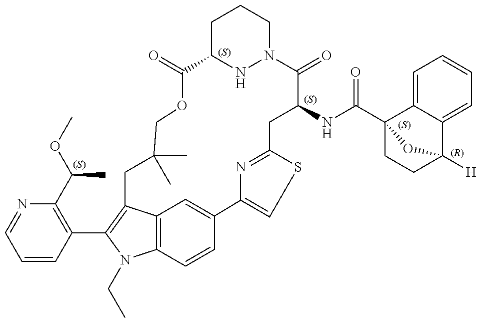 | DA 245 | 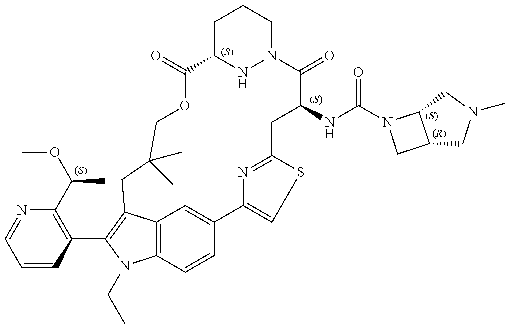 |
| DA 70 | 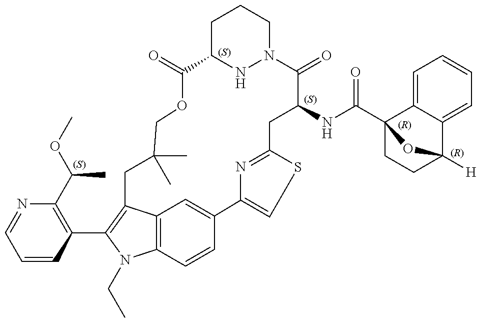 | DA 246 | 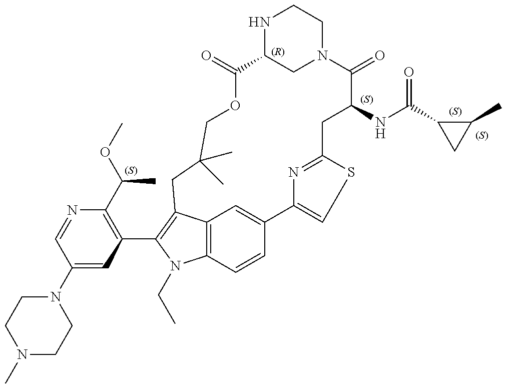 |
| DA 71 | 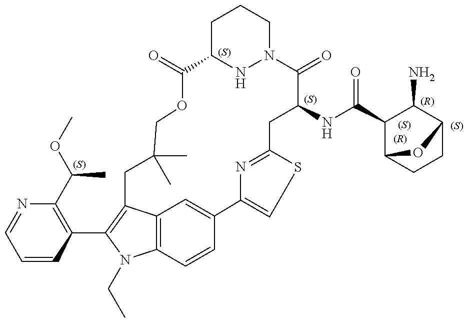 | DA 247 | 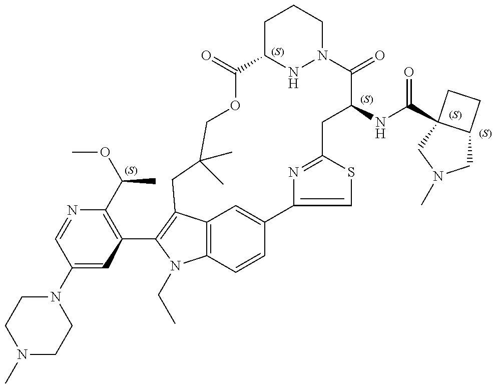 |
| DA 72 | 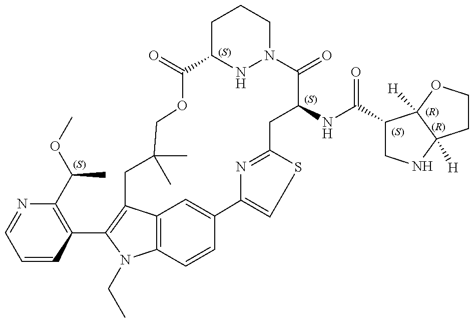 | DA 248 | 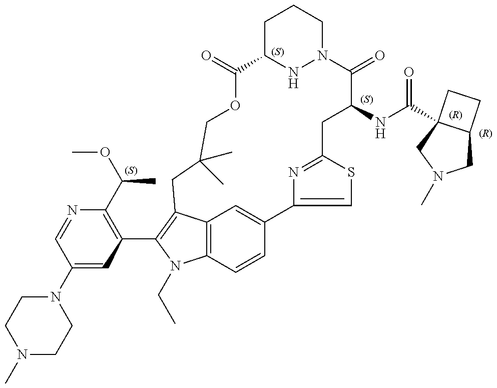 |
| DA 73 | 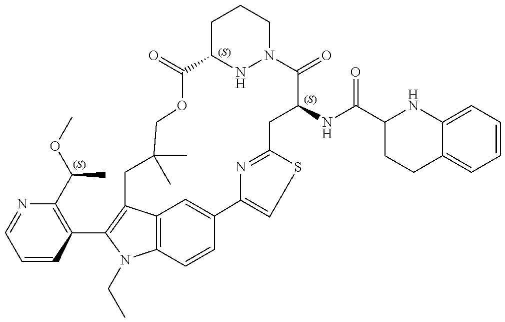 | DA 249 | 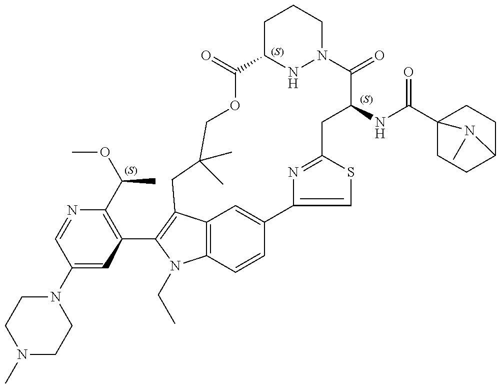 |
| DA 74 | 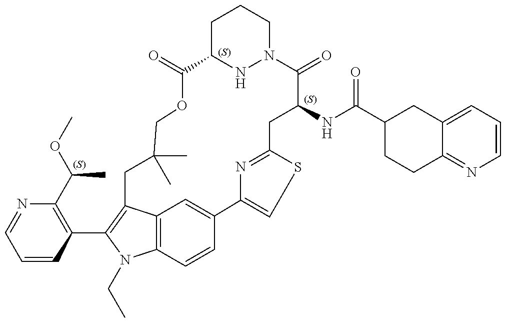 | DA 250 | 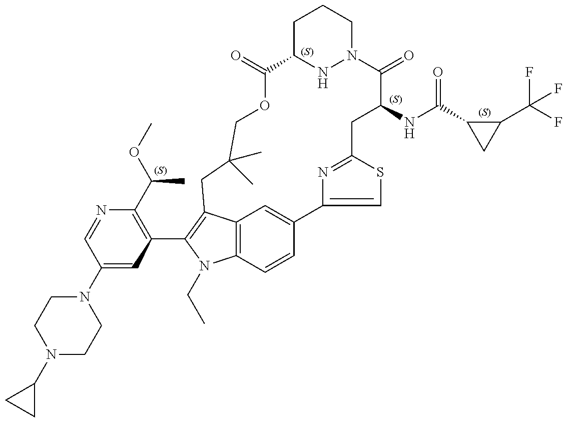 |
| DA 75 | 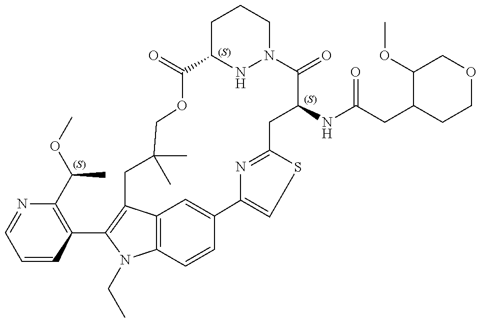 | DA 251 | 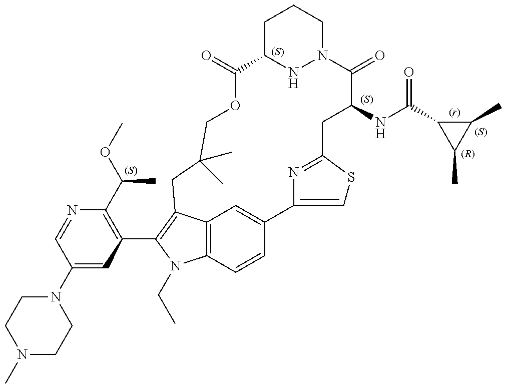 |
| DA 76 | 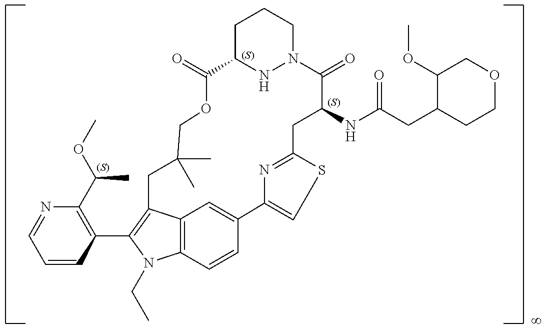 | DA 252 | 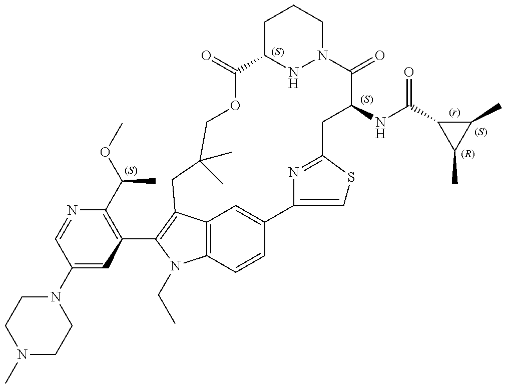 |
| DA 77 | 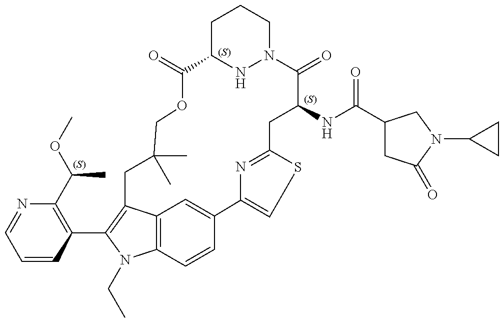 | DA 253 | 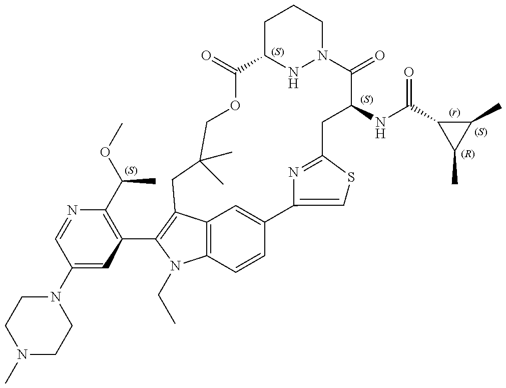 |
| DA 78 | 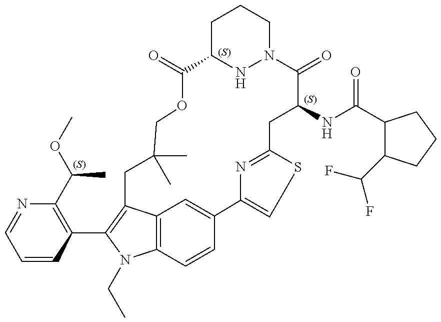 | DA 254 | 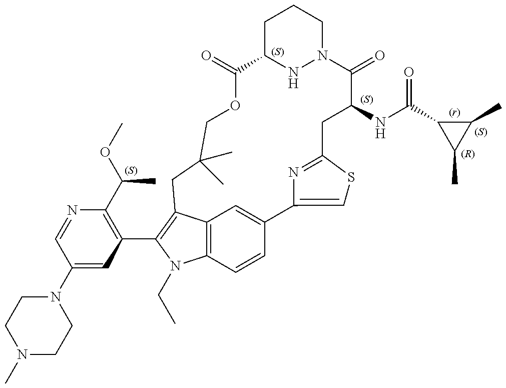 |
| DA 79 | 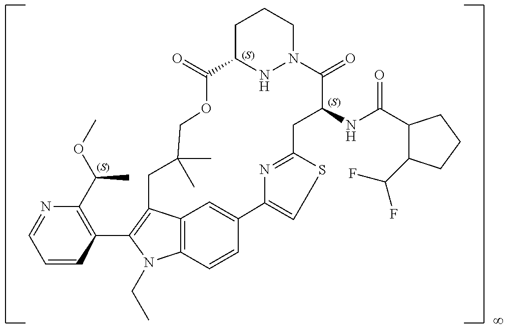 | DA 255 | 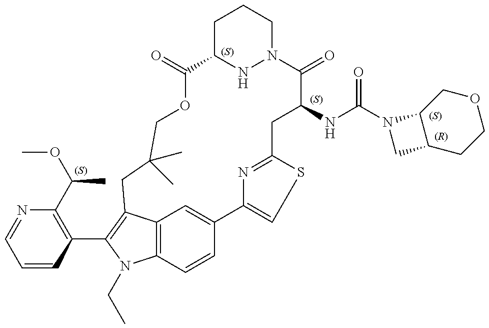 |
| DA 80 | 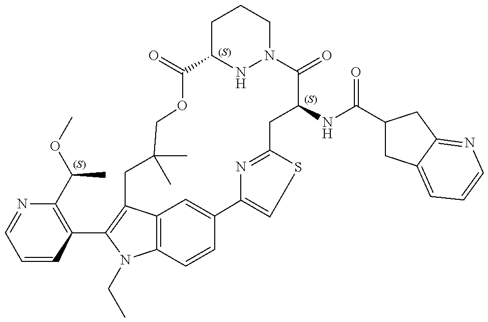 | DA 256 | 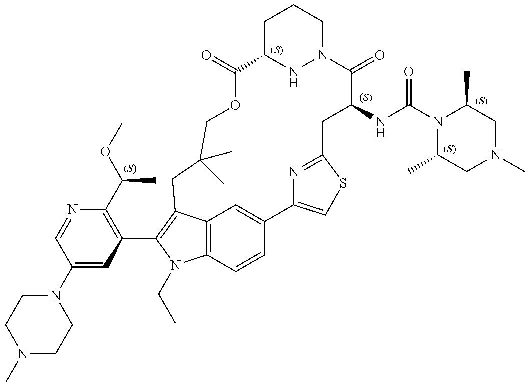 |
| DA 81 | 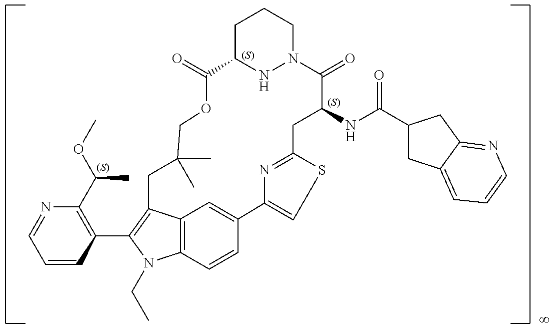 | DA 257 | 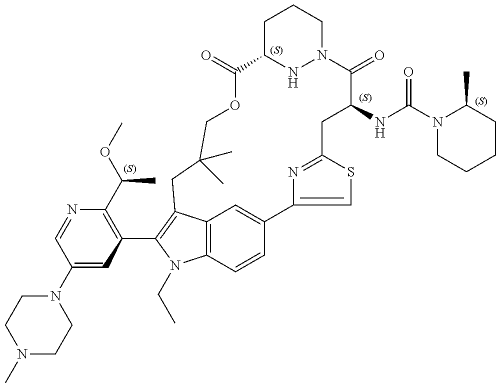 |
| DA 82 | 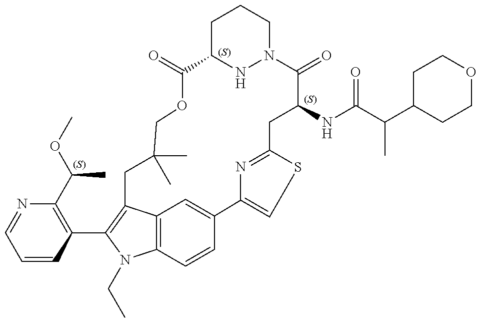 | DA 258 | 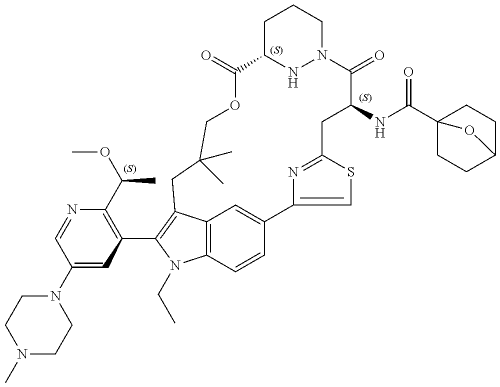 |
| DA 83 | 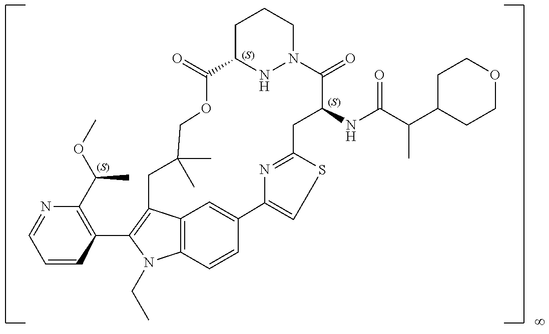 | DA 259 | 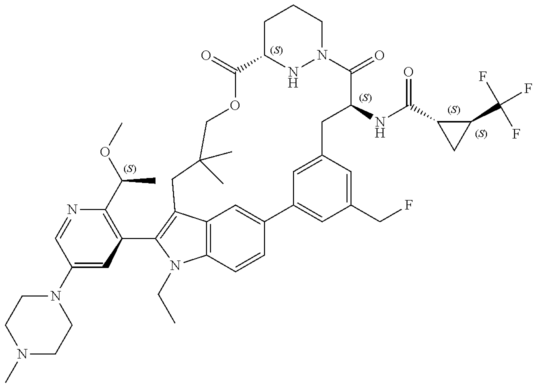 |
| DA 84 | 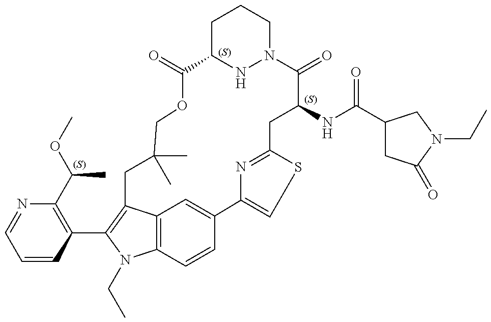 | DA 260 | 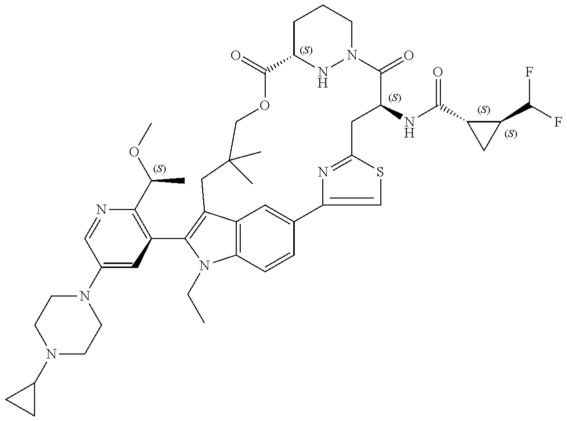 |
| DA 85 | 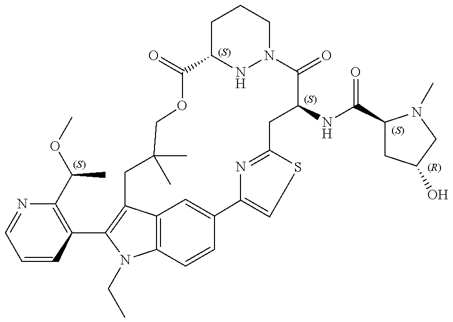 | DA 261 | 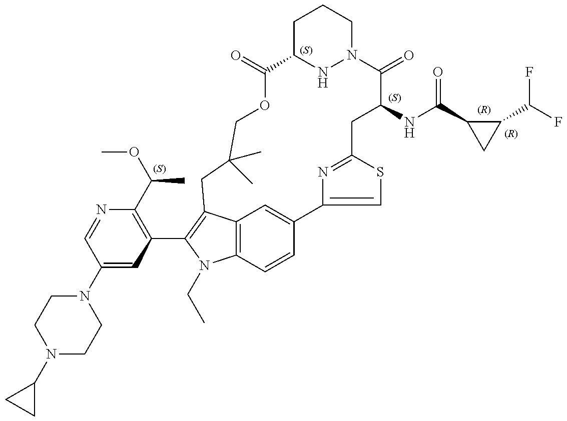 |
| DA 86 | 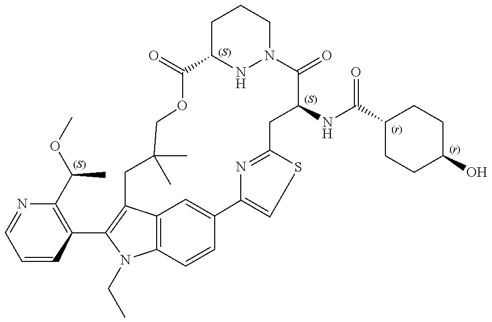 | DA 262 | 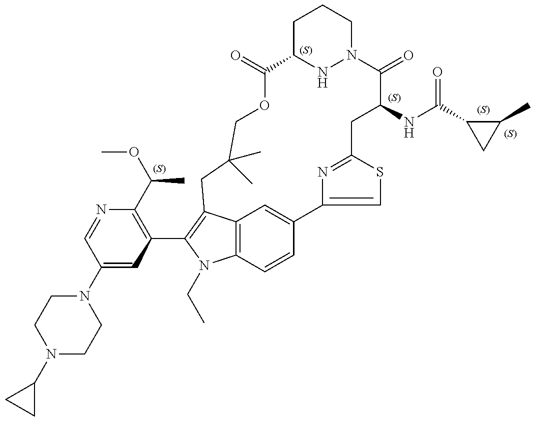 |
| DA 87 | 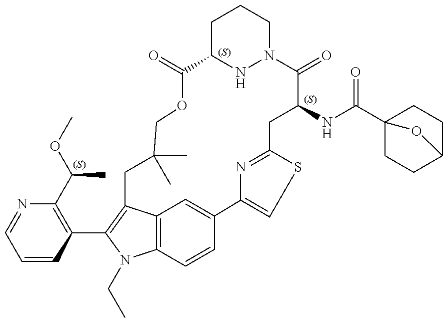 | DA 263 | 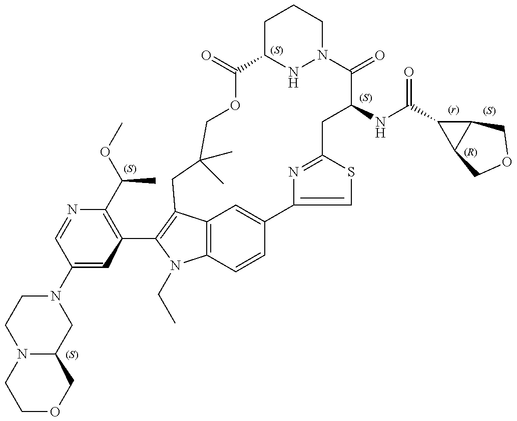 |
| DA 88 | 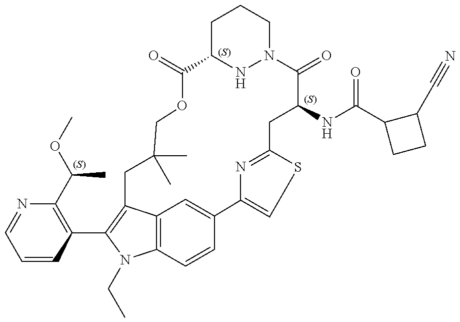 | DA 264 | 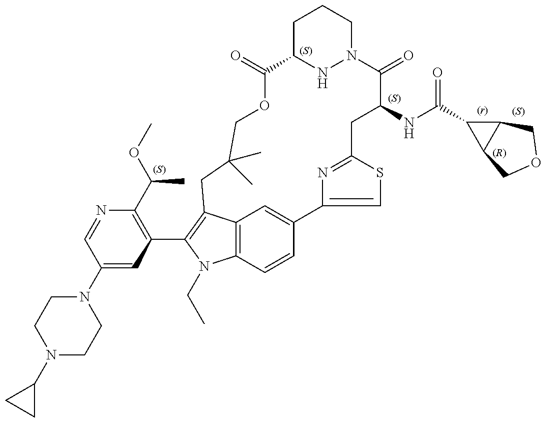 |
| DA 89 | 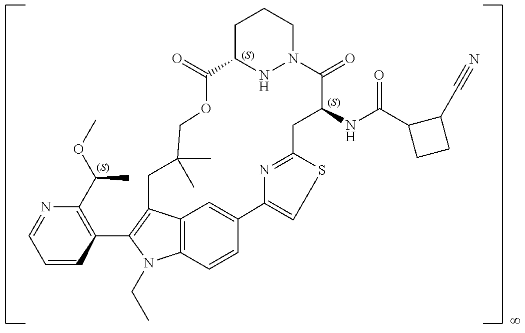 | DA 265 | 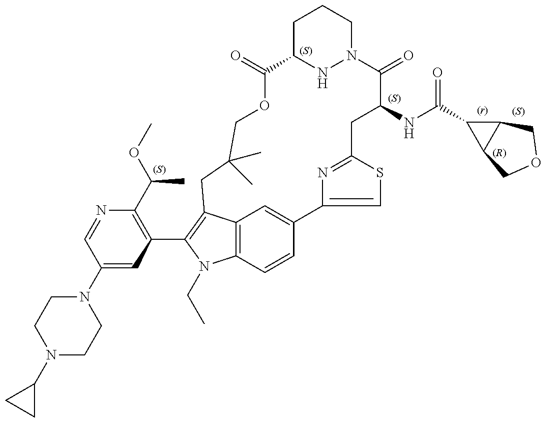 |
| DA 90 | 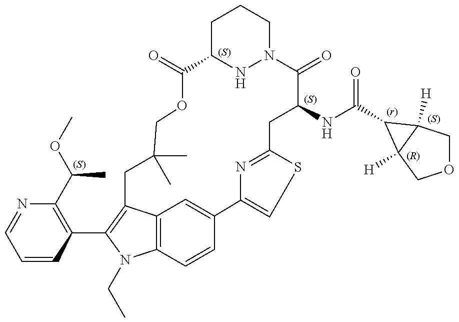 | DA 266 | 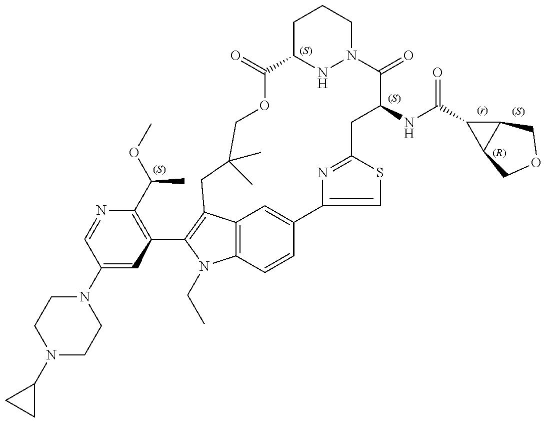 |
| DA 91 | 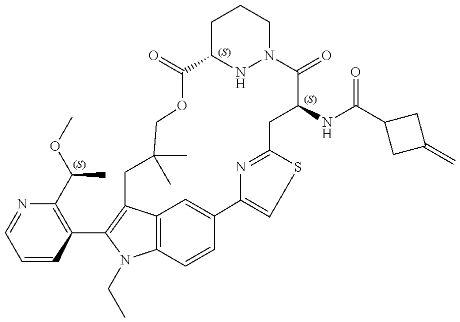 | DA 267 | 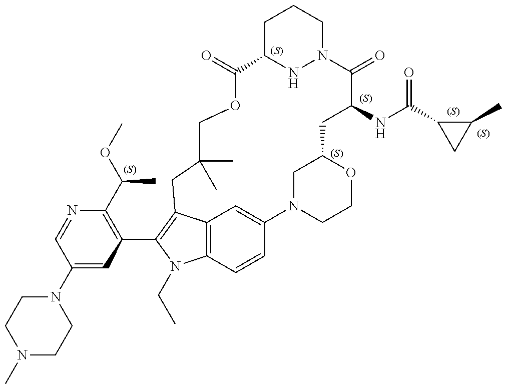 |
| DA 92 | 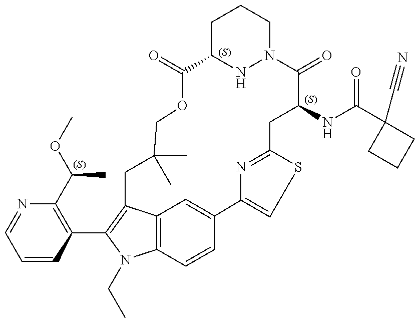 | DA 268 | 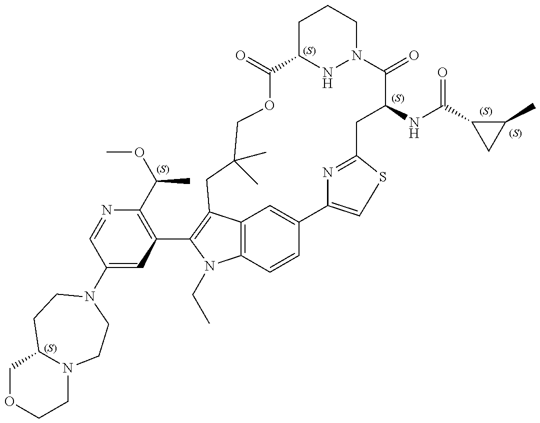 |
| DA 93 | 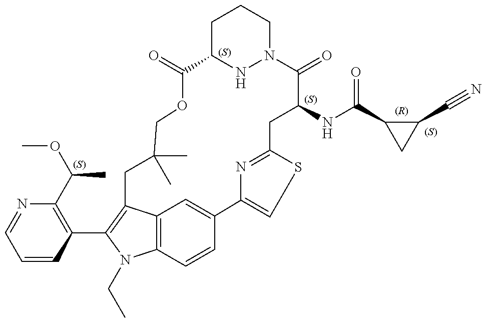 | DA 269 | 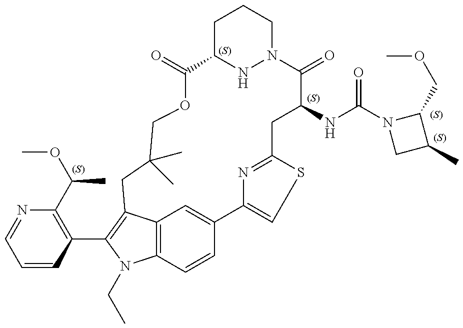 |
| DA 94 | 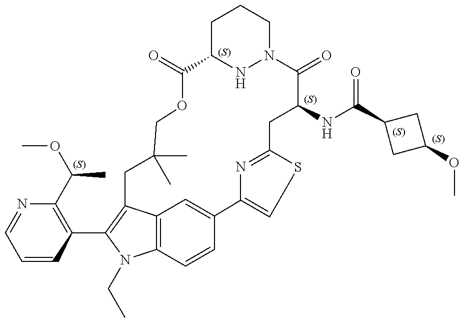 | DA 270 | 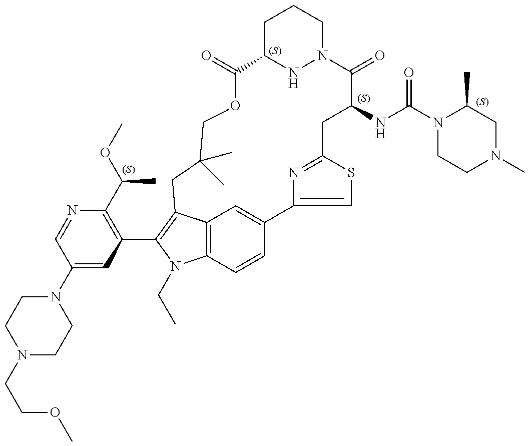 |
| DA 95 | 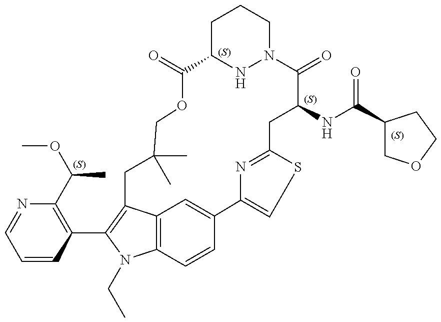 | DA 271 | 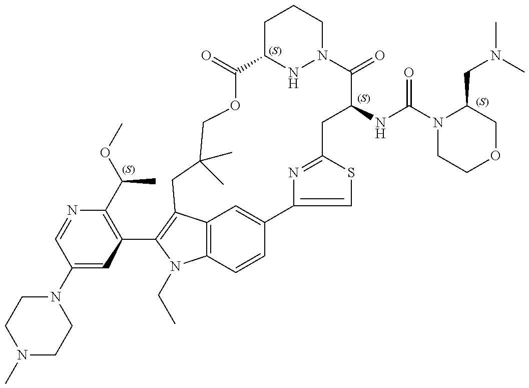 |
| DA 96 | 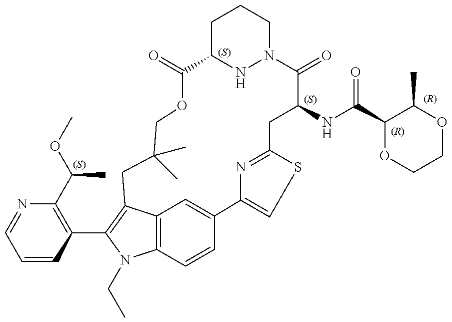 | DA 272 | 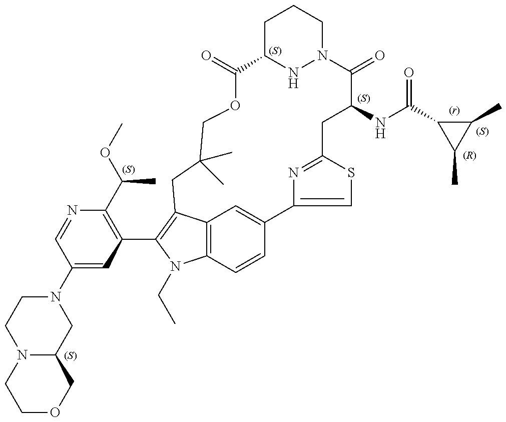 |
| DA 97 | 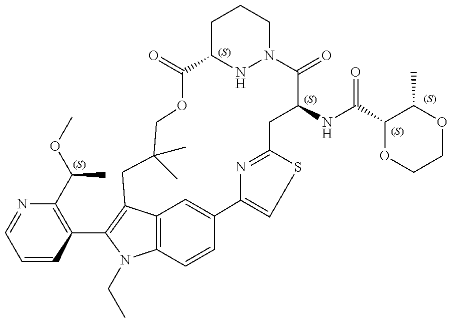 | DA 273 | 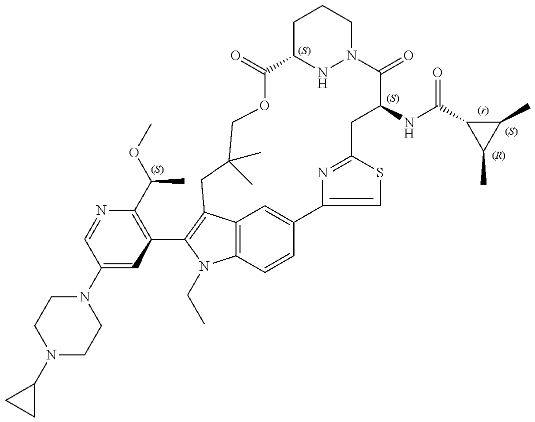 |
| DA 98 | 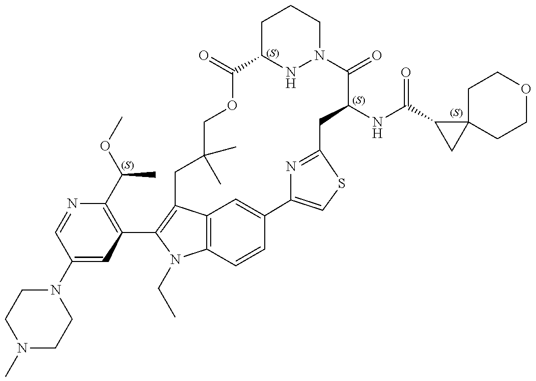 | DA 274 | 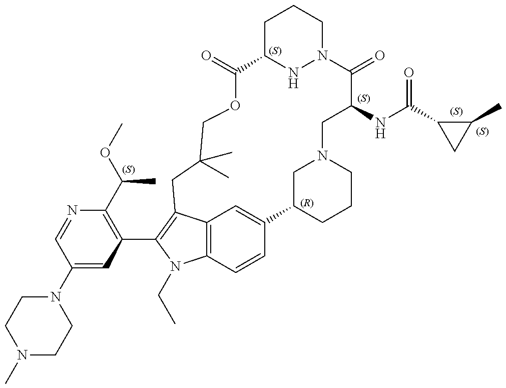 |
| DA 99 | 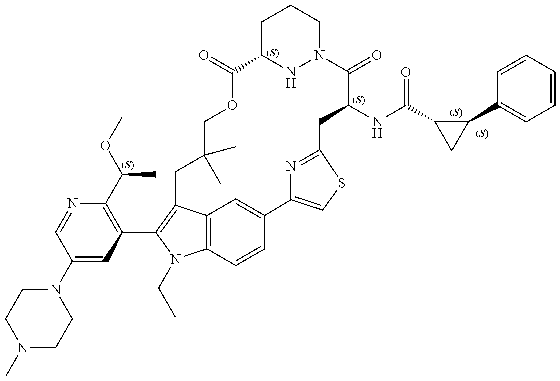 | DA 275 | 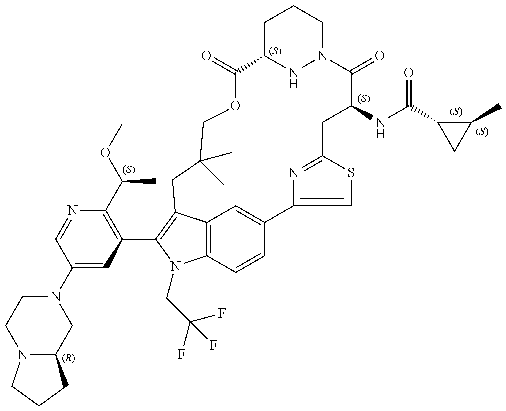 |
| DA 100 | 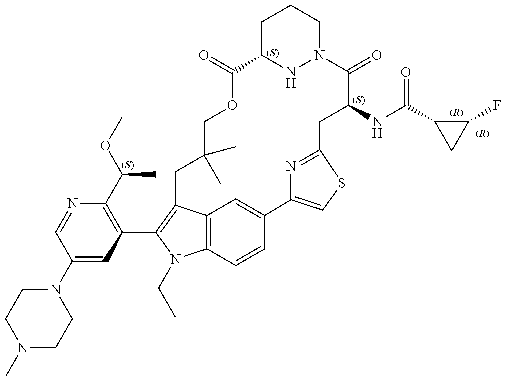 | DA 276 | 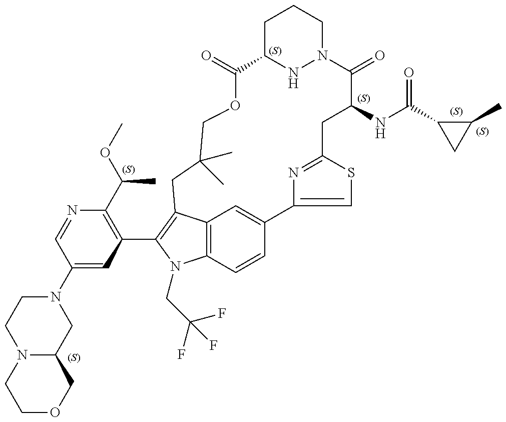 |
| DA 101 | 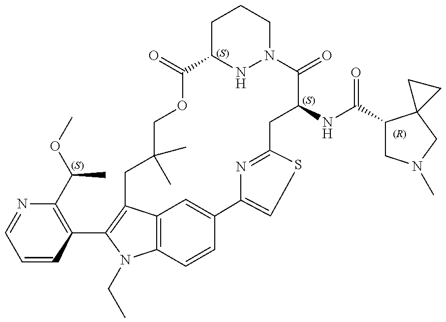 | DA 277 | 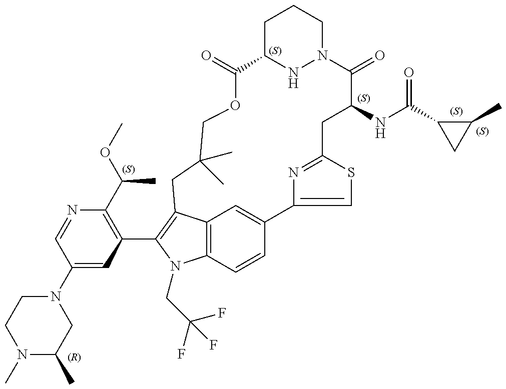 |
| DA 102 | 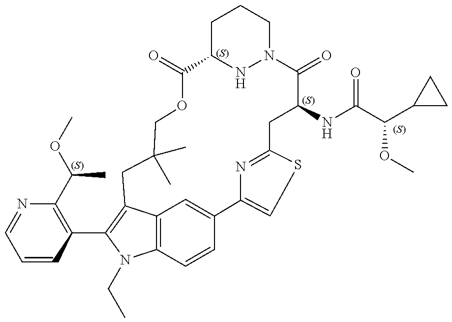 | DA 278 | 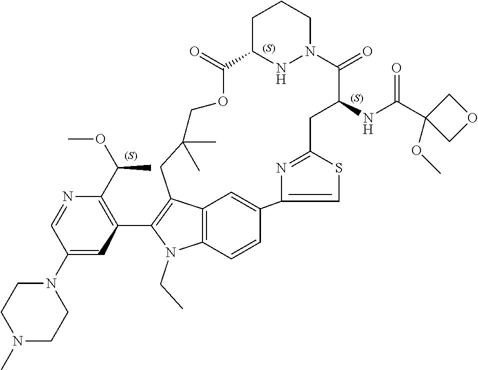 |
| DA 103 | 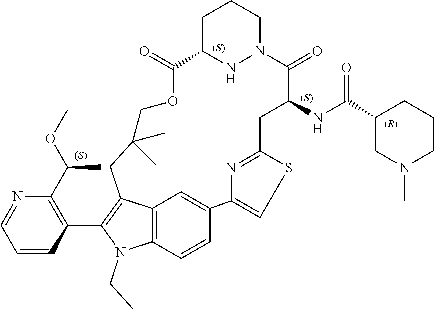 | DA 279 | 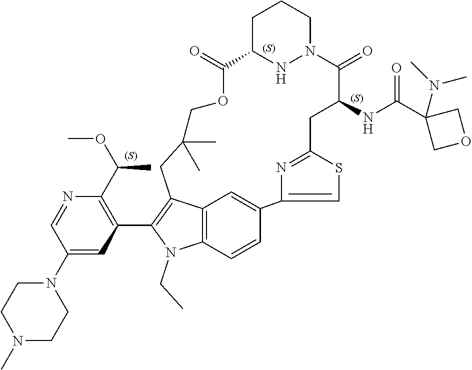 |
| DA 104 | 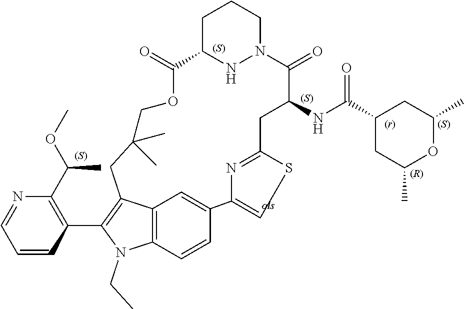 | DA 280 | 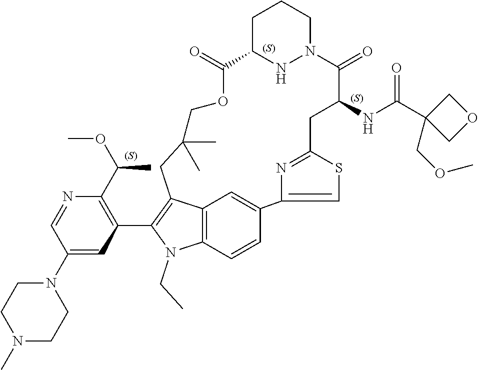 |
| DA 105 | 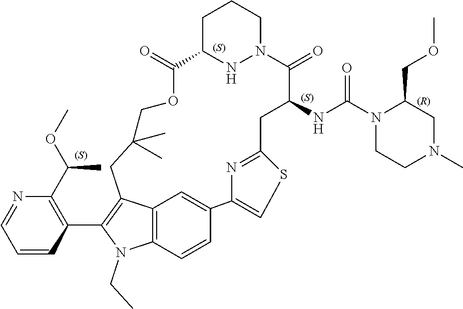 | DA 281 | 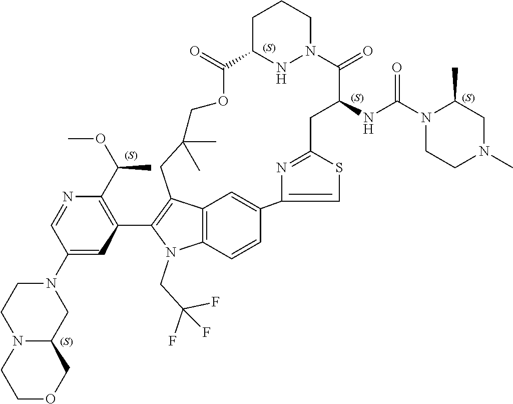 |
| DA 106 | 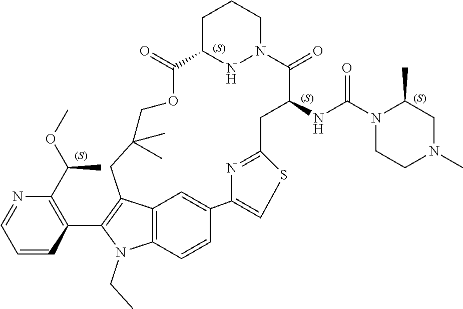 | DA 282 | 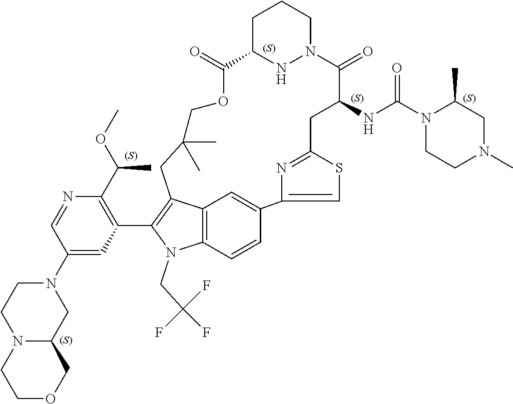 |
| DA 107 | 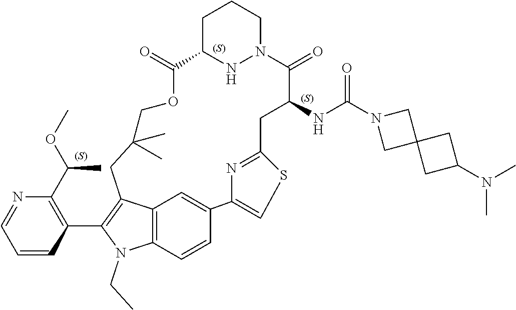 | DA 283 | 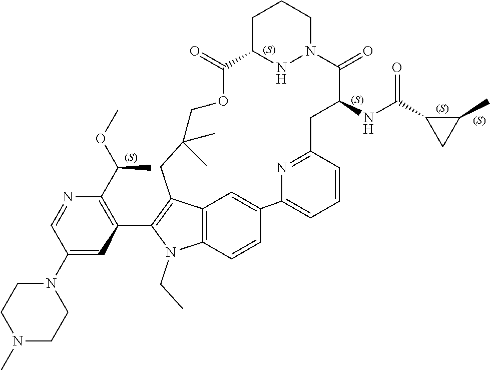 |
| DA 108 | 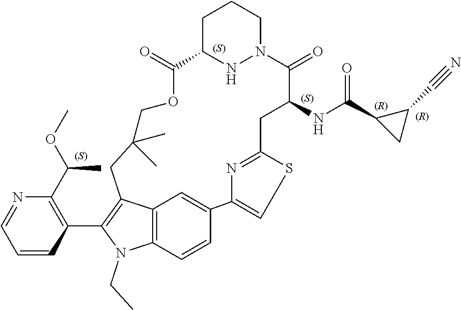 | DA 284 | 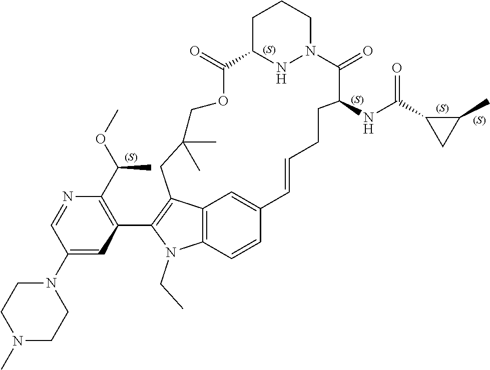 |
| DA 109 | 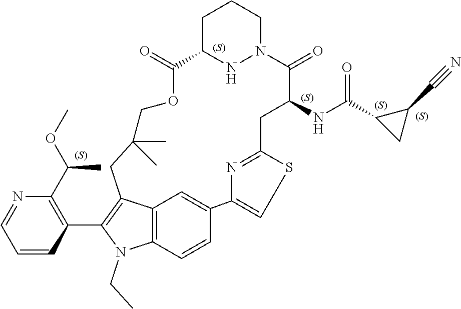 | DA 285 | 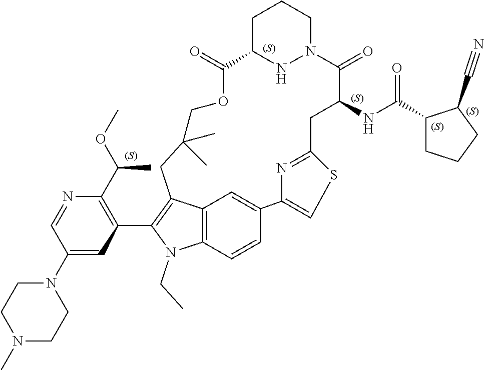 |
| DA 110 | 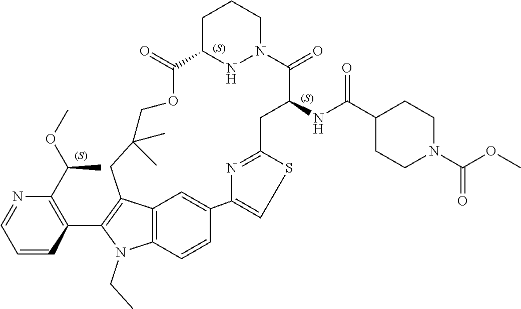 | DA 286 | 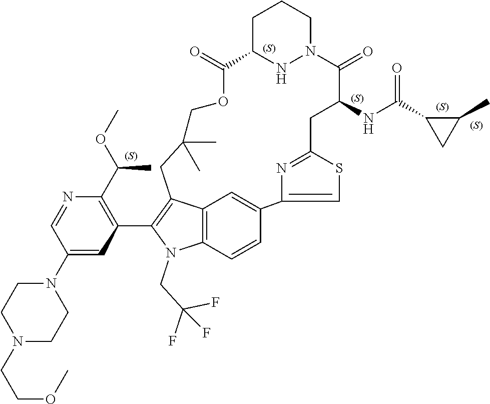 |
| DA 111 | 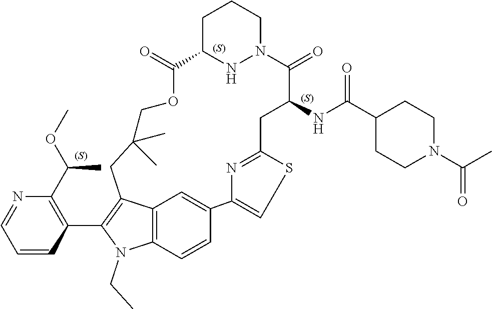 | DA 287 | 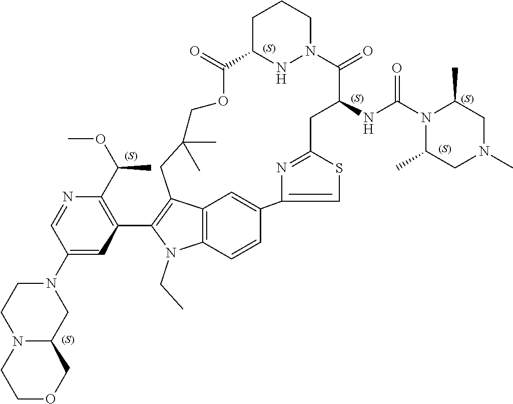 |
| DA 112 | 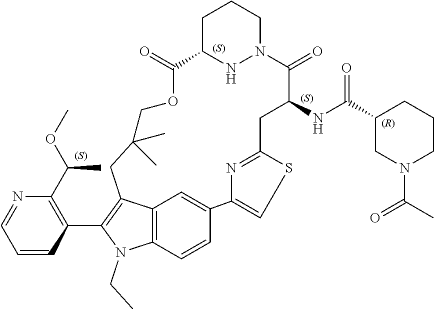 | DA 288 | 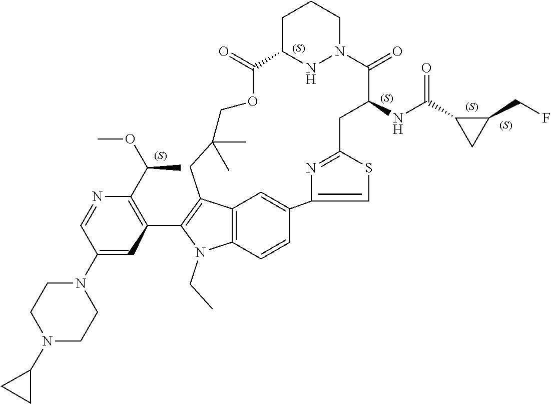 |
| DA 113 | 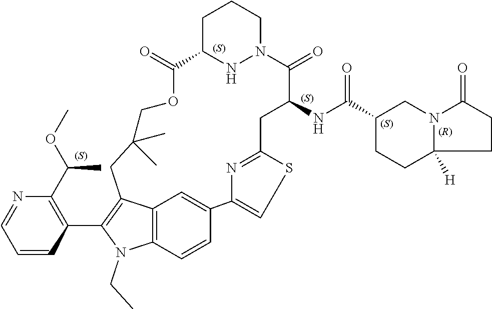 | DA 289 | 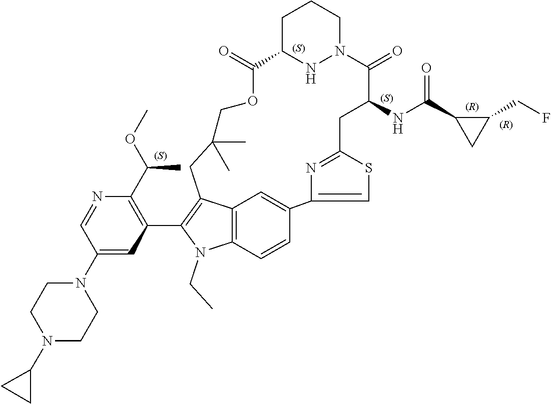 |
| DA 114 | 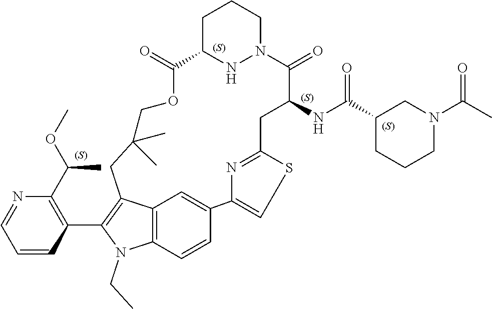 | DA 290 | 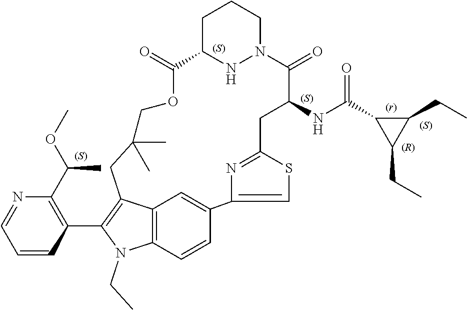 |
| DA 115 | 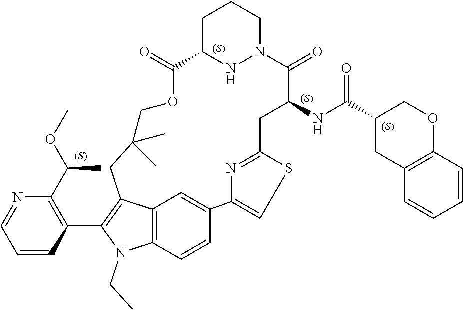 | DA 291 | 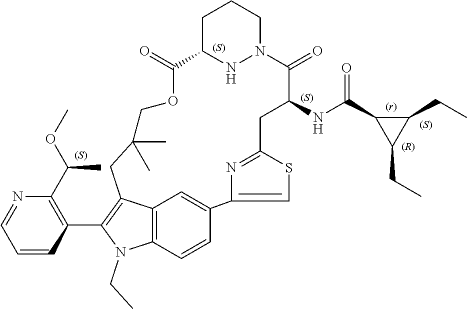 |
| DA 116 | 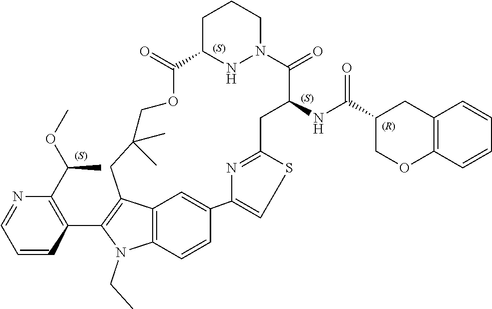 | DA 292 | 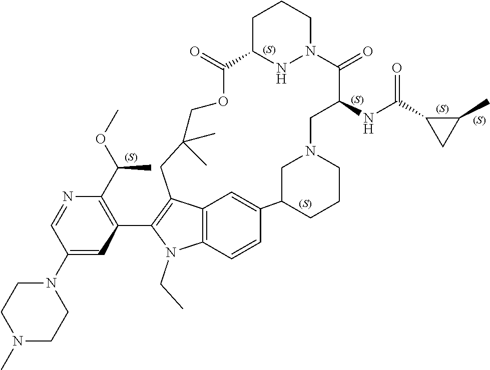 |
| DA 117 | 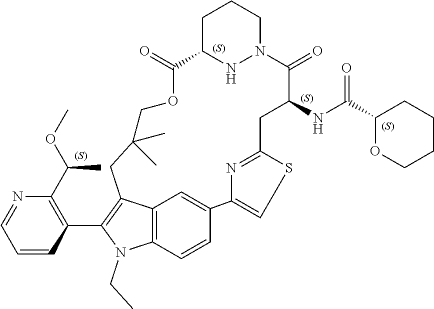 | DA 293 | 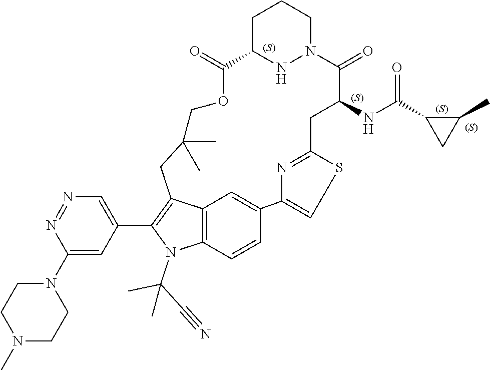 |
| DA 118 | 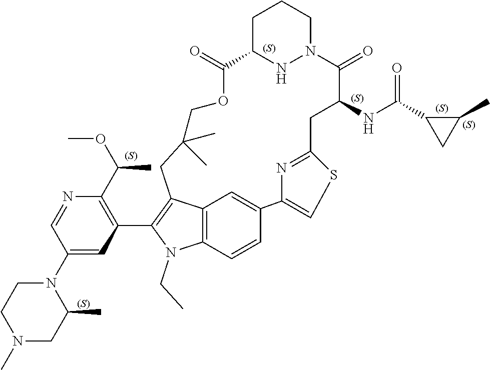 | DA 294 | 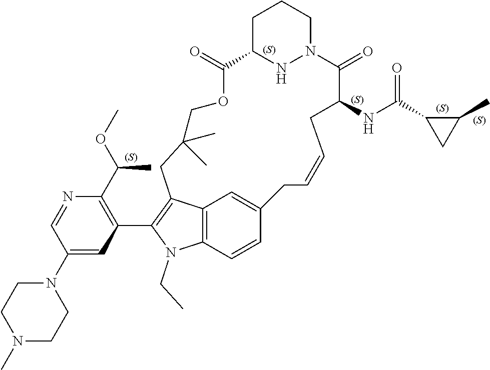 |
| DA 119 | 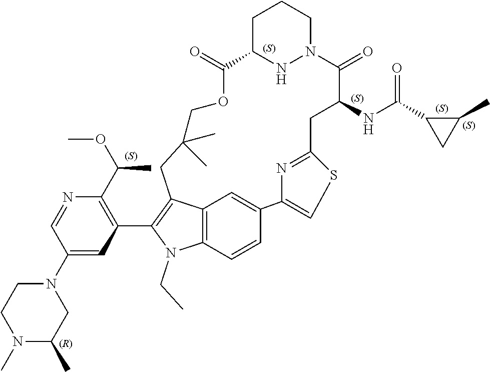 | DA 295 | 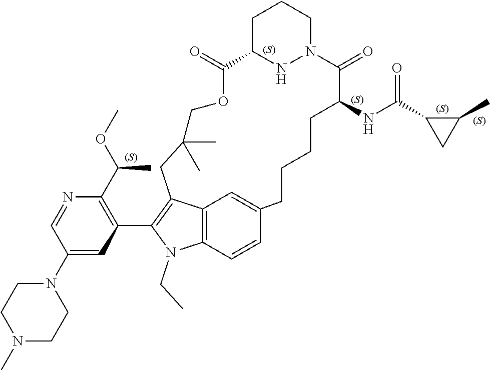 |
| DA 120 | 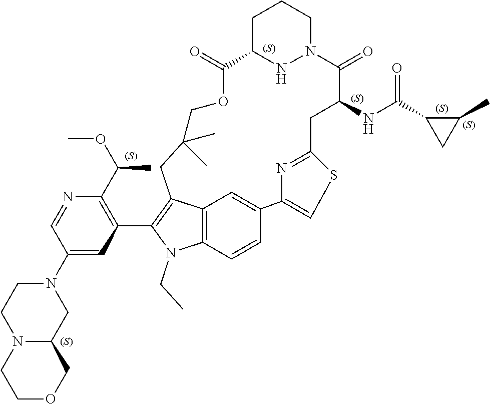 | DA 296 | 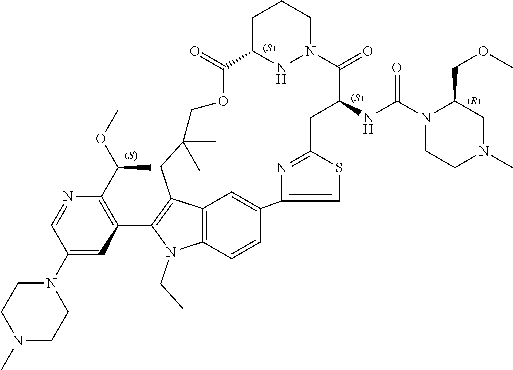 |
| DA 121 | 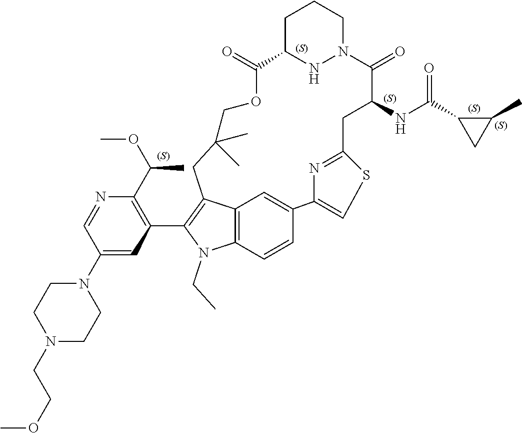 | DA 297 | 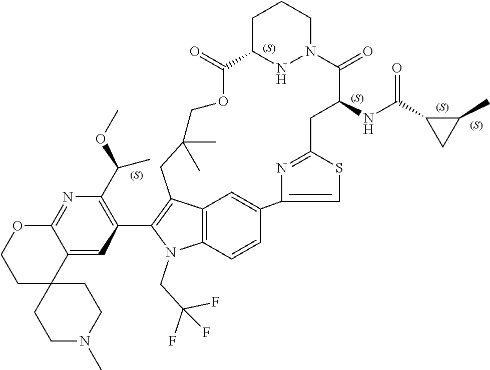 |
| DA 122 | 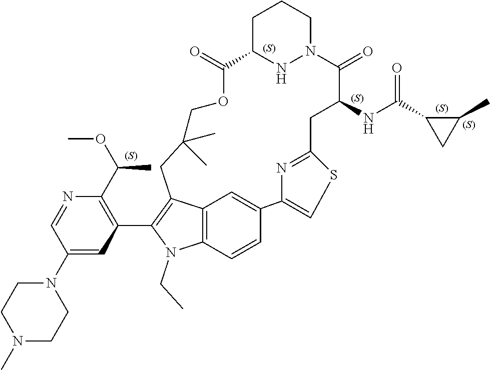 | DA 298 | 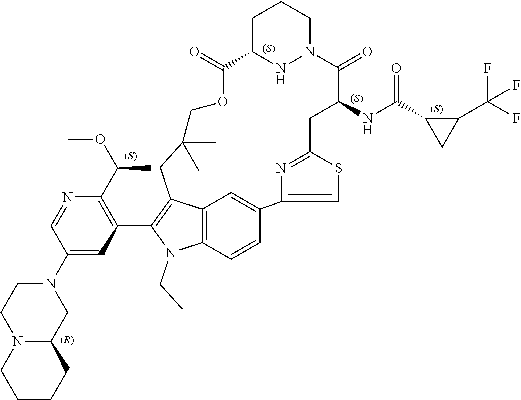 |
| DA 123 | 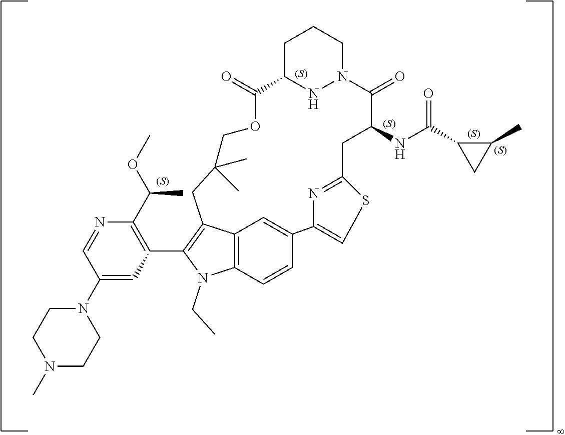 | DA 299 | 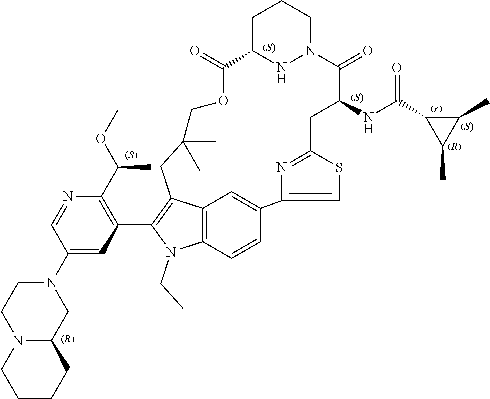 |
| DA 124 | 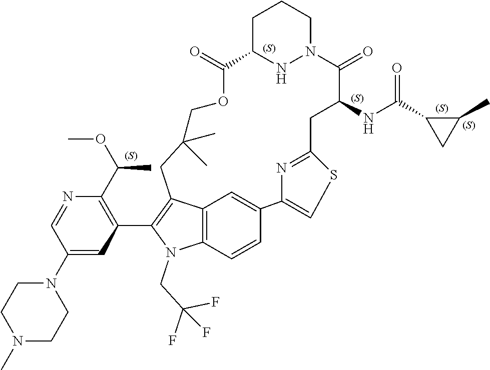 | DA 300 | 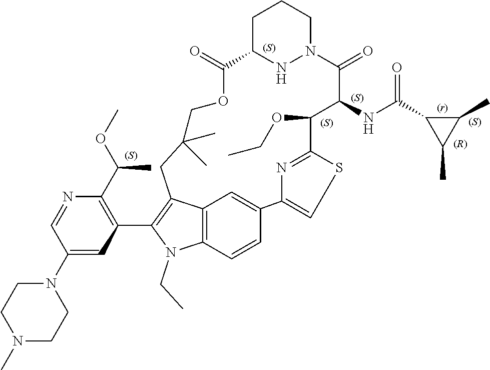 |
| DA 125 | 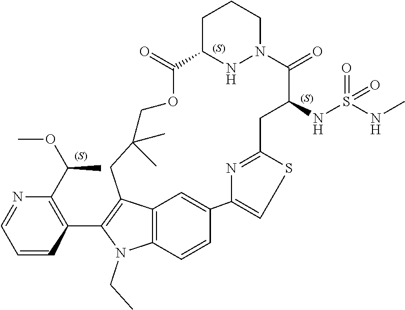 | DA 301 | 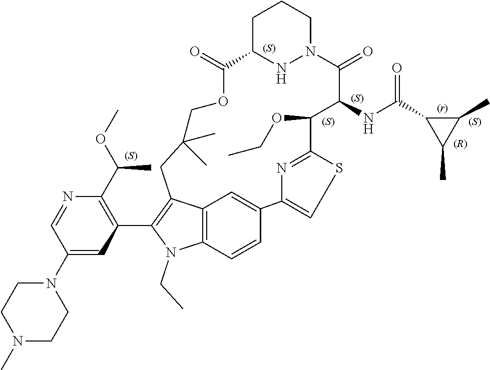 |
| DA 126 | 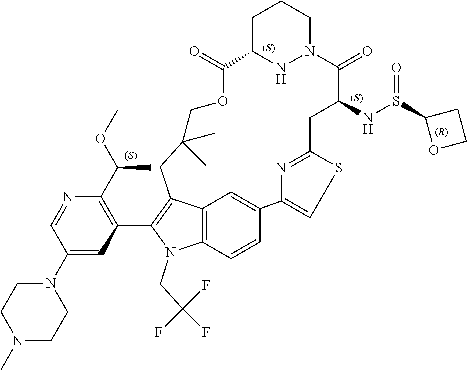 | DA 302 | 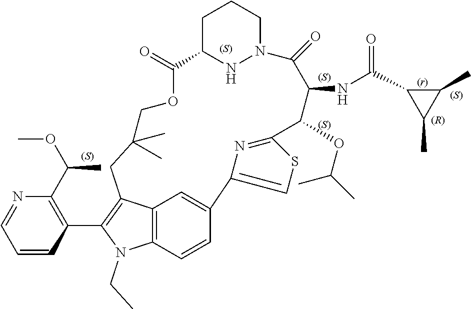 |
| DA 127 | 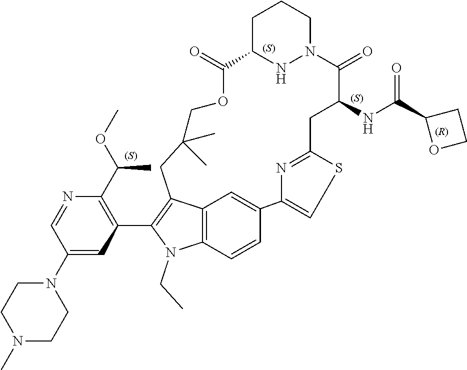 | DA 303 | 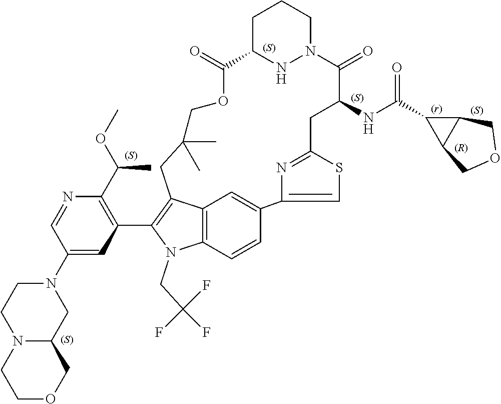 |
| DA 128 | 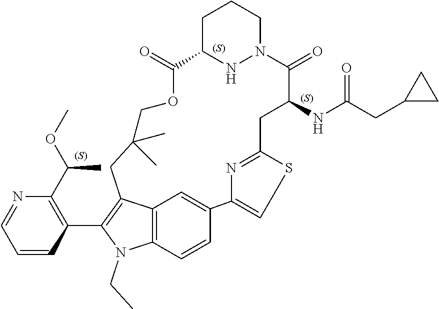 | DA 304 | 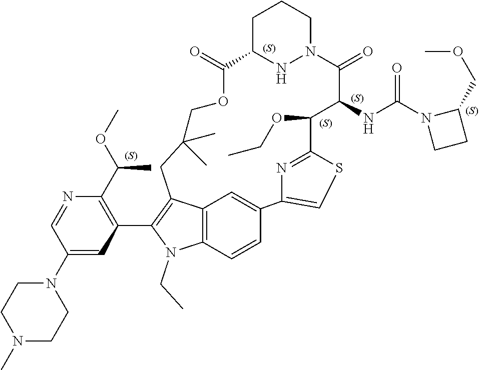 |
| DA 129 | 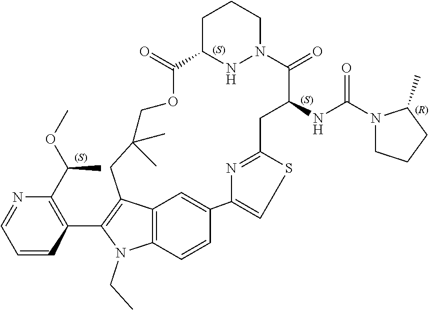 | DA 305 | 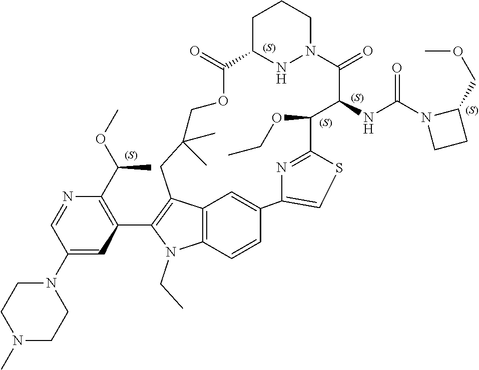 |
| DA 130 | 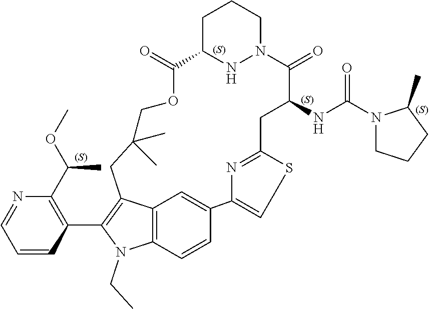 | DA 306 | 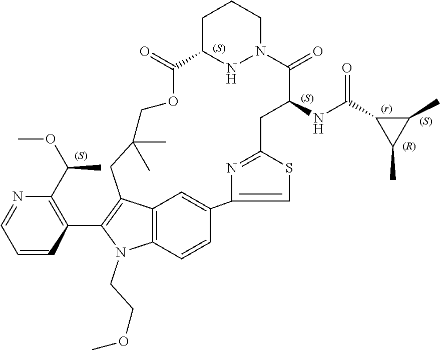 |
| DA 131 | 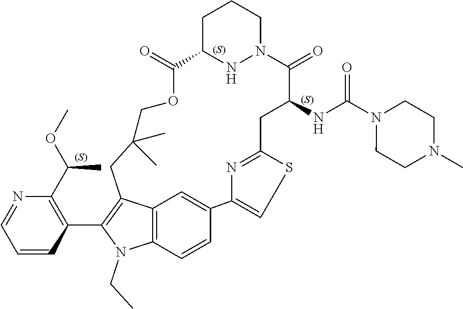 | DA 307 | 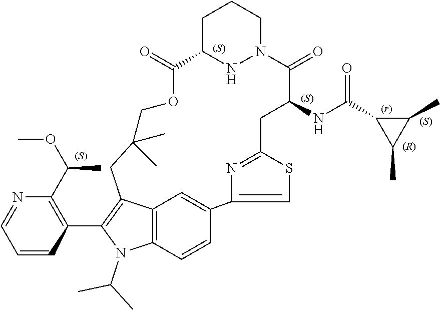 |
| DA 132 | 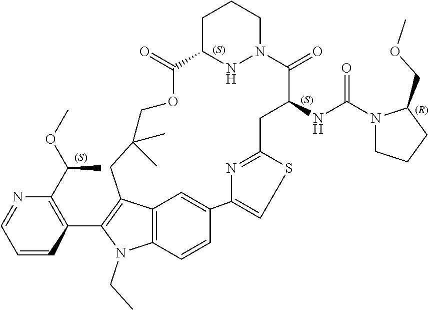 | DA 308 | 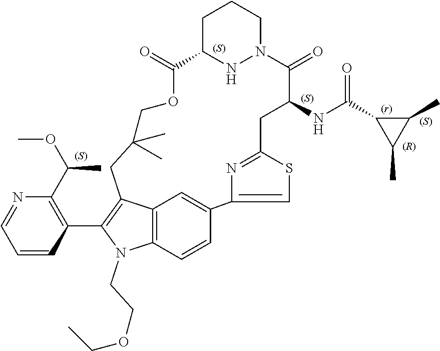 |
| DA 133 | 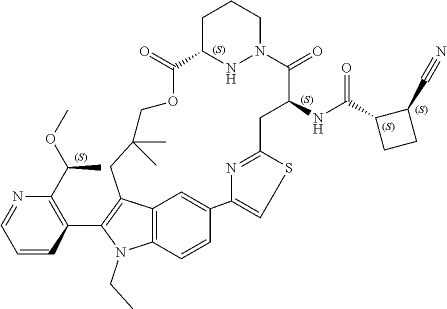 | DA 309 | 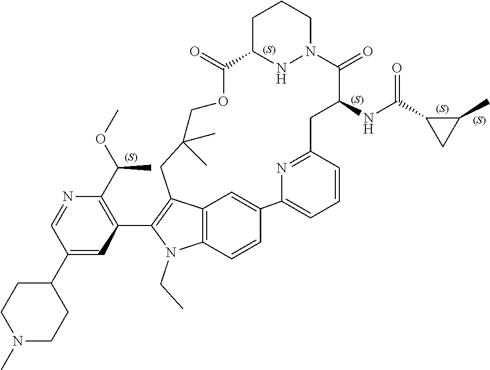 |
| DA 134 | 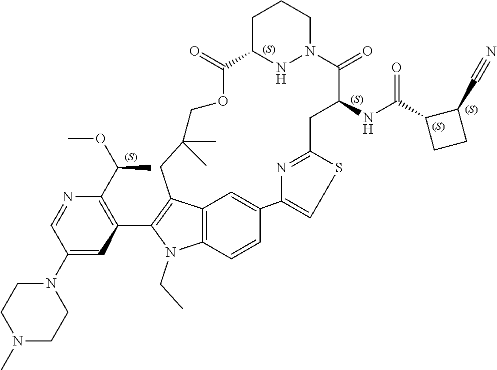 | DA 311 | 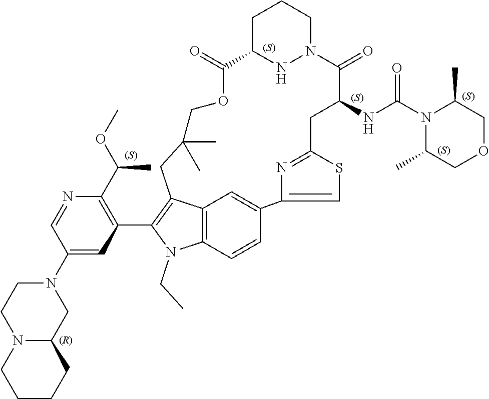 |
| DA 135 | 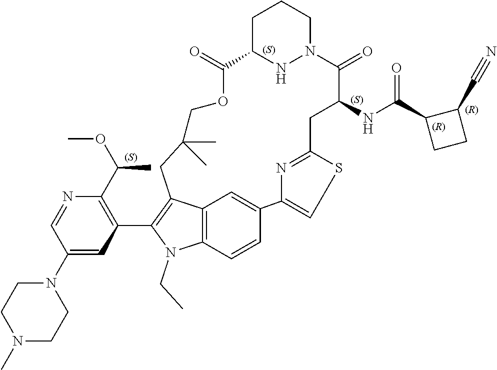 | DA 312 | 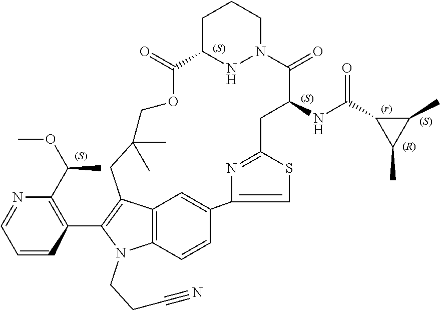 |
| DA 136 | 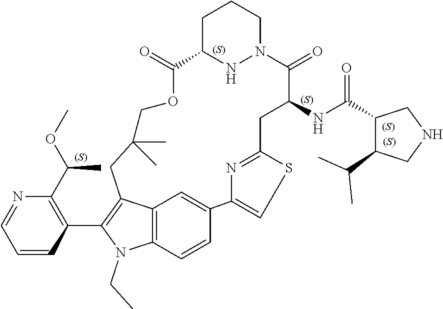 | DA 313 | 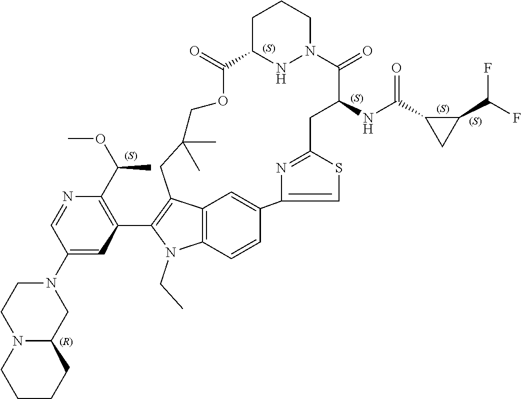 |
| DA 137 | 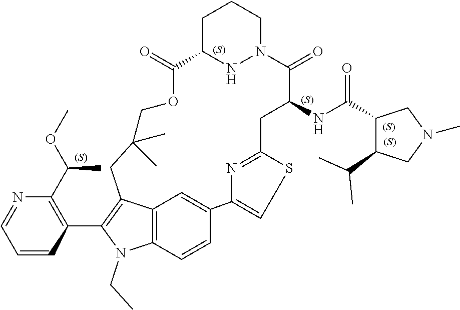 | DA 314 | 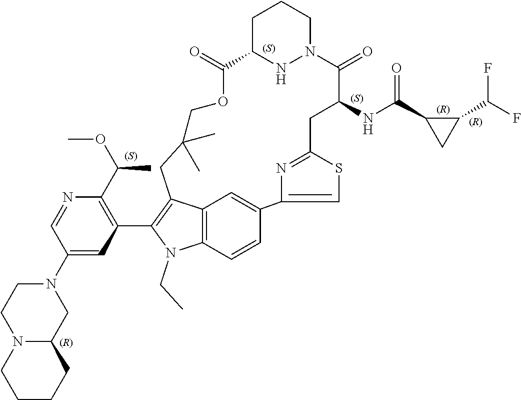 |
| DA 138 | 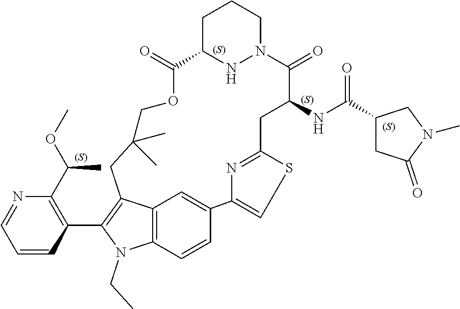 | DA 315 | 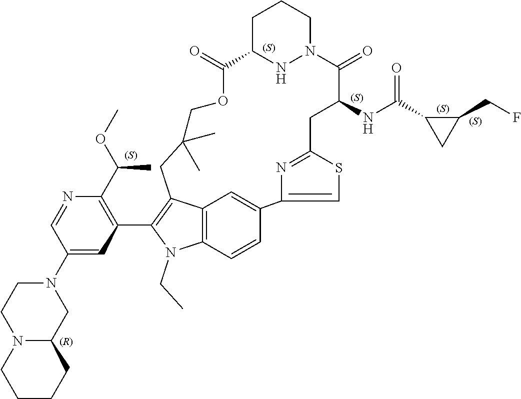 |
| DA 139 | 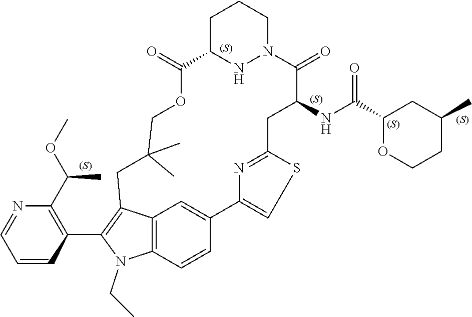 | DA 316 | 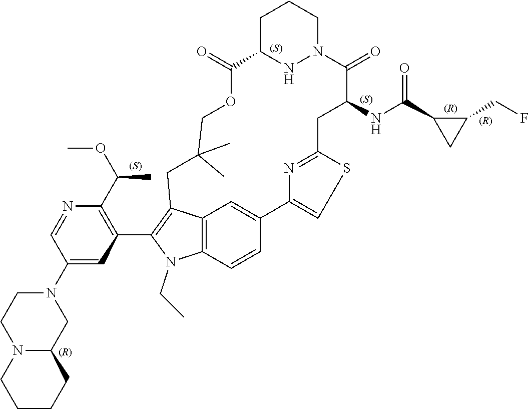 |
| DA 140 | 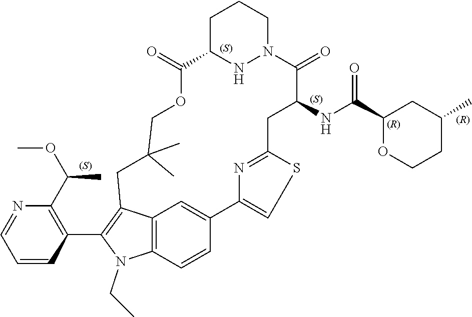 | DA 317 | 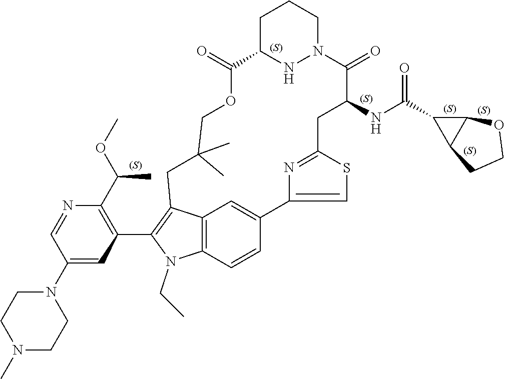 |
| DA 141 | 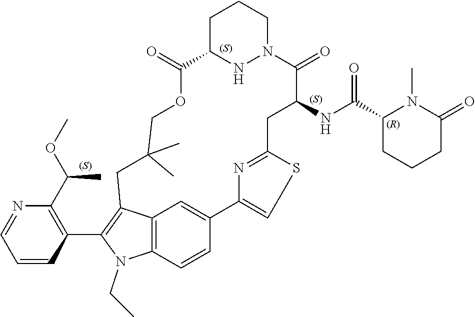 | DA 318 | 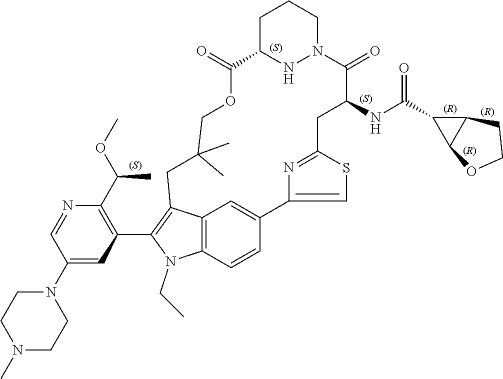 |
| DA 142 | 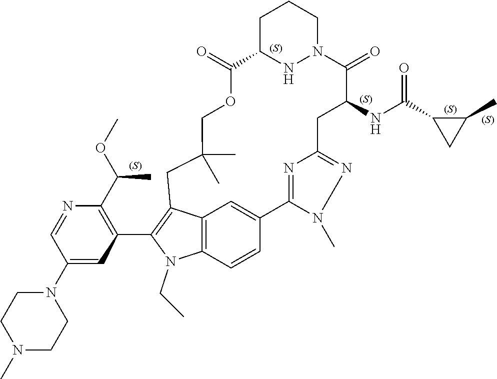 | DA 319 | 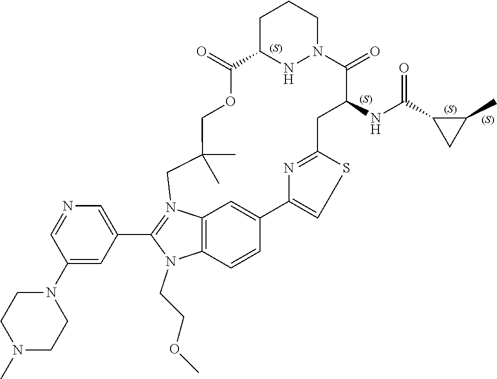 |
| DA 143 | 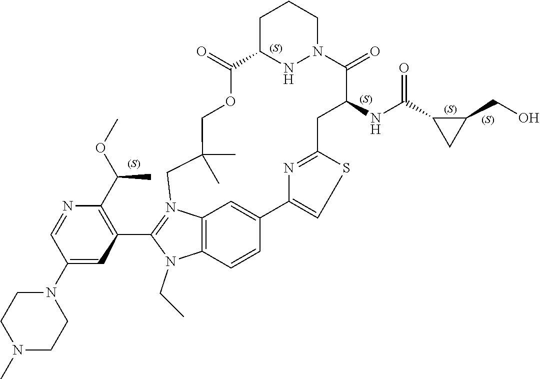 | DA 320 | 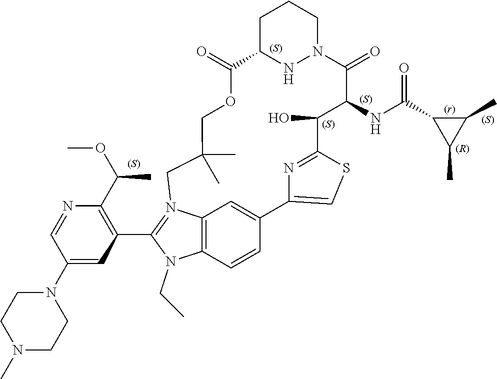 |
| DA 144 | 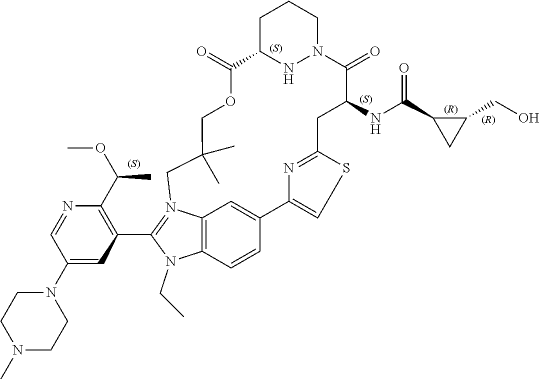 | DA 321 | 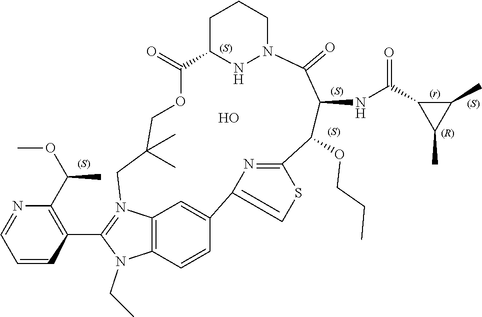 |
| DA 145 | 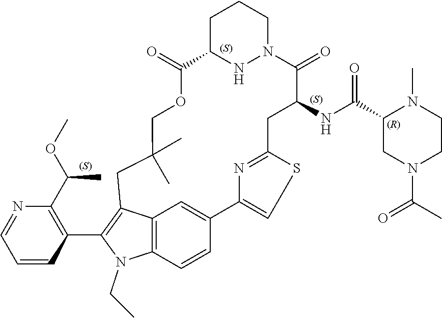 | DA 322 | 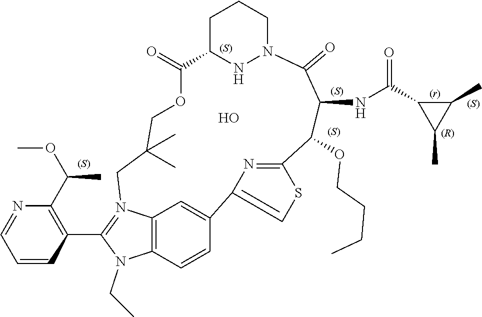 |
| DA 146 | 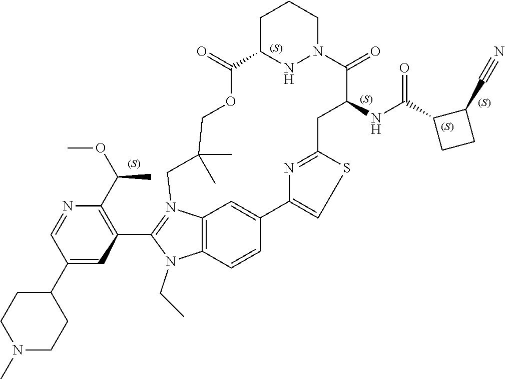 | DA 323 | 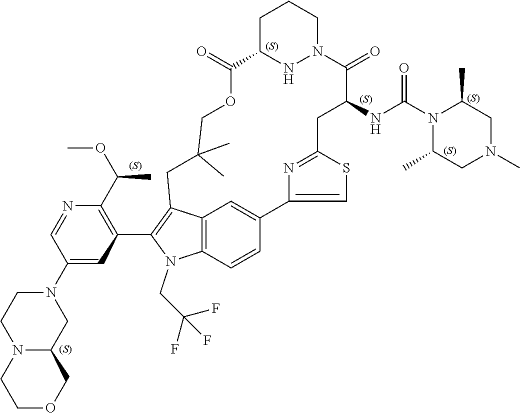 |
| DA 147 | 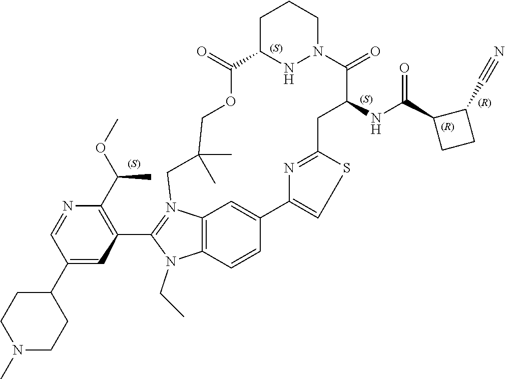 | DA 324 | 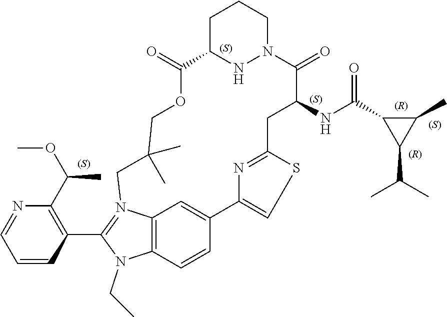 |
| DA 148 | 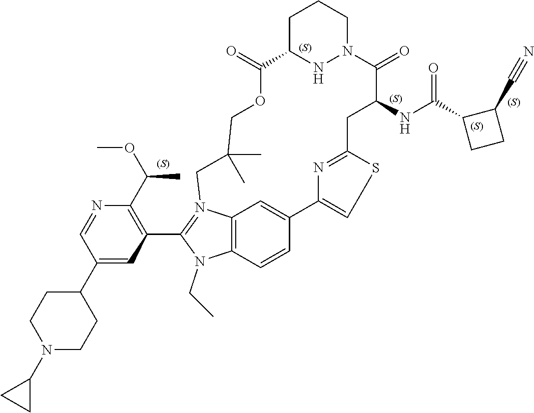 | DA 325 | 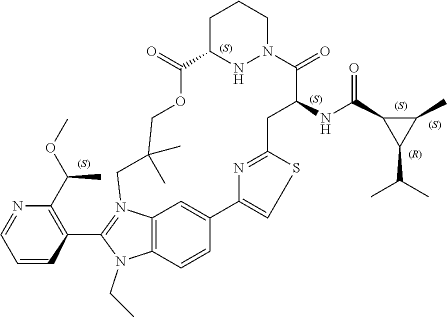 |
| DA 149 | 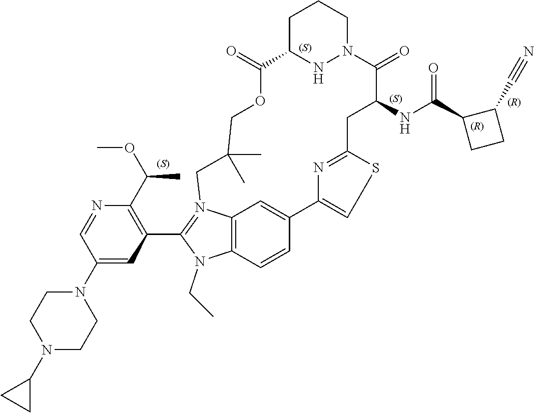 | DA 326 | 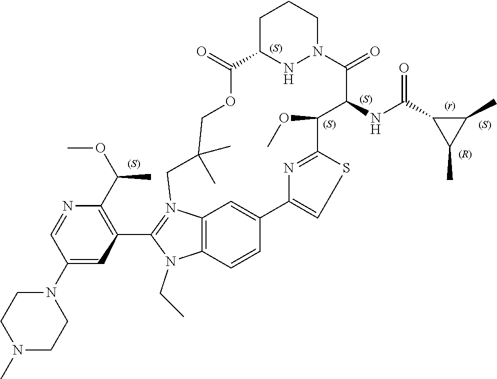 |
| DA 150 | 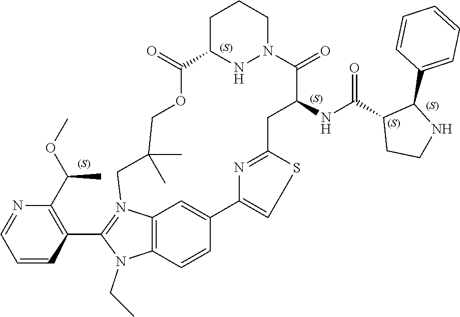 | DA 327 | 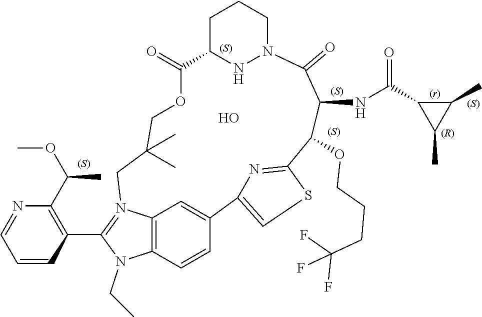 |
| DA 151 | 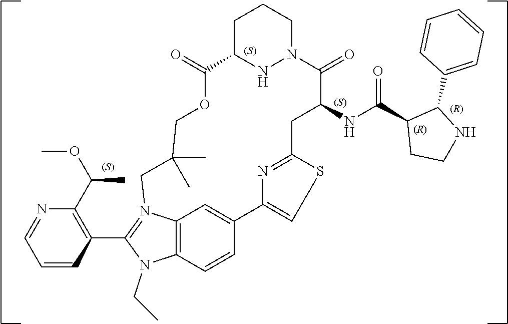 | DA 328 | 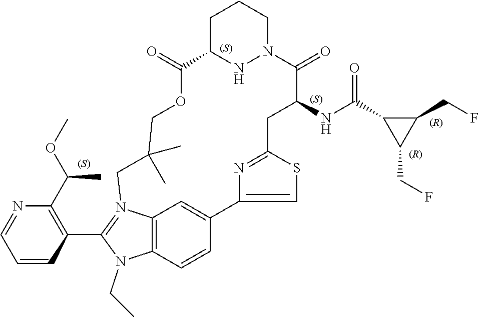 |
| DA 152 | 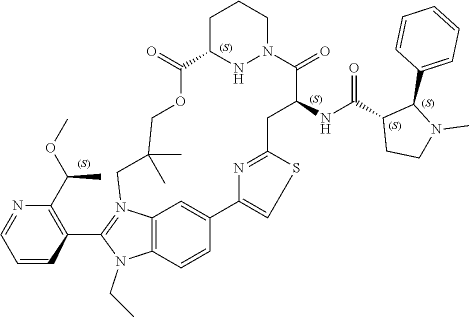 | DA 329 | 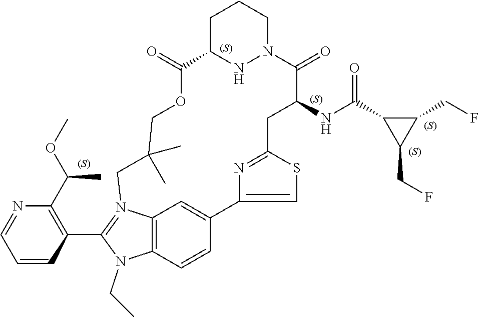 |
| DA 153 | 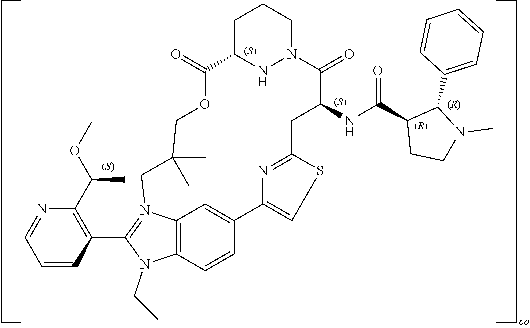 | DA 330 | 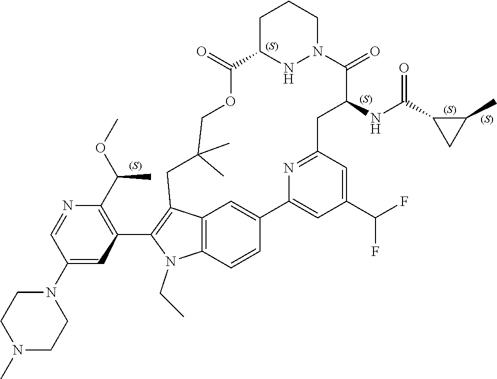 |
| DA 154 | 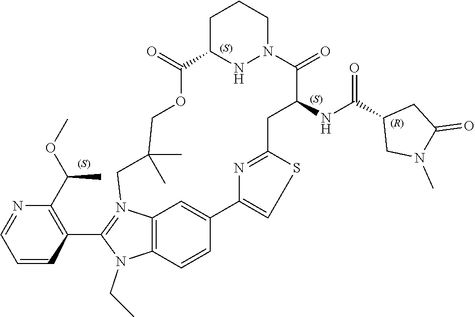 | DA 331 | 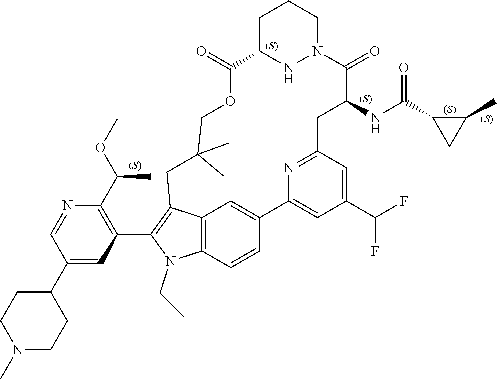 |
| DA 155 | 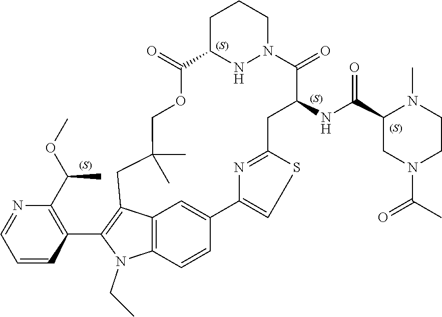 | DA 332 | 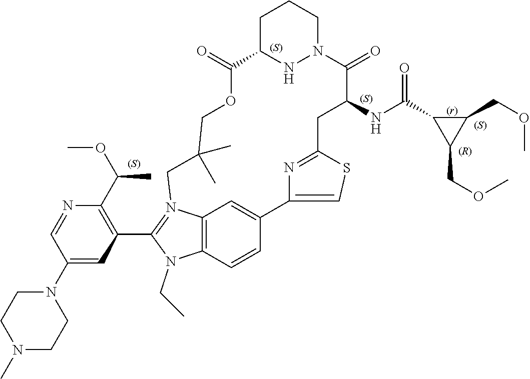 |
| DA 156 | 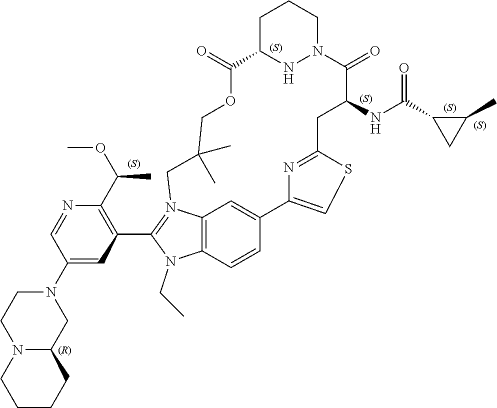 | DA 333 | 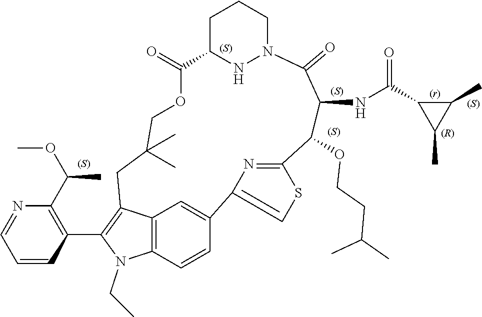 |
| DA 157 | 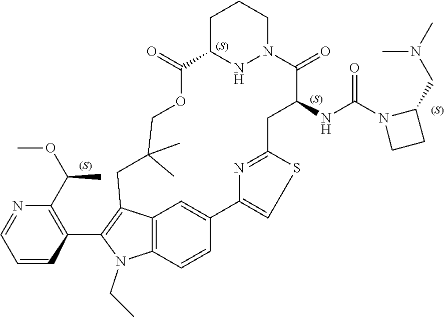 | DA 334 | 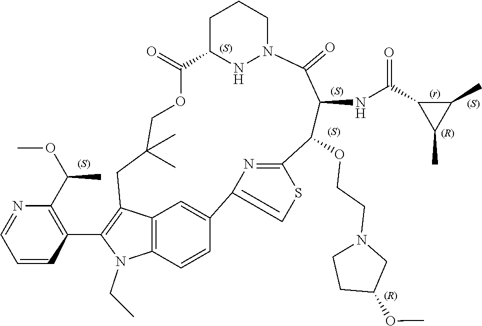 |
| DA 158 | 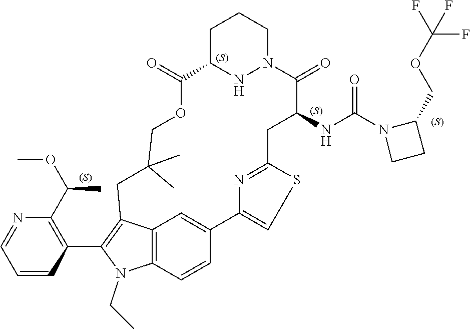 | DA 335 | 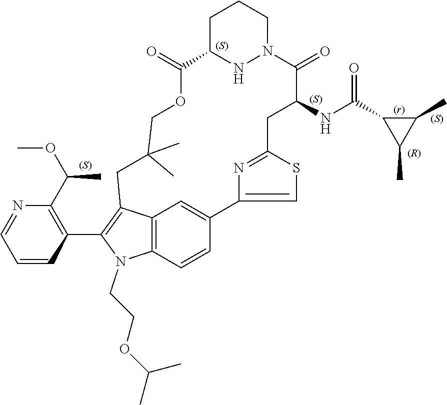 |
| DA 159 | 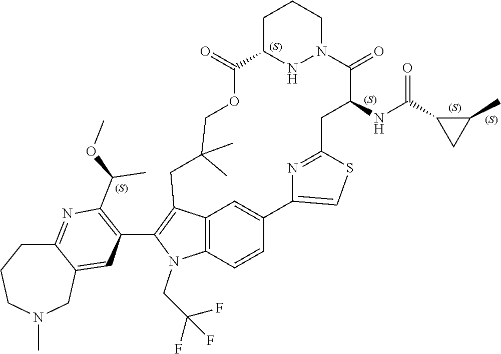 | DA 336 | 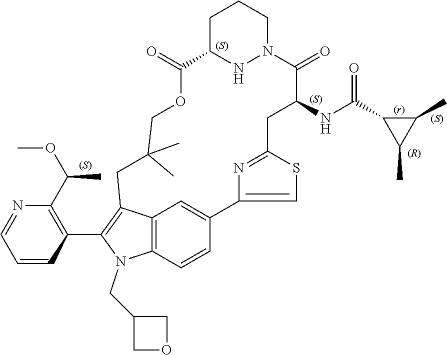 |
| DA 160 | 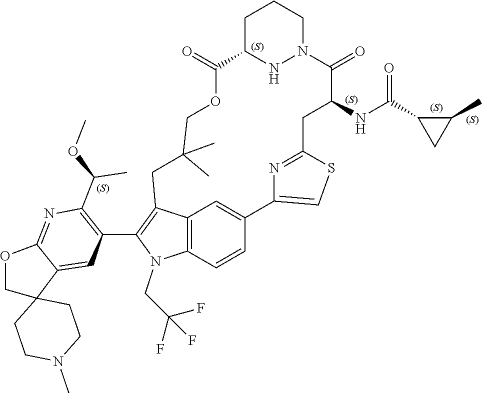 | DA 337 | 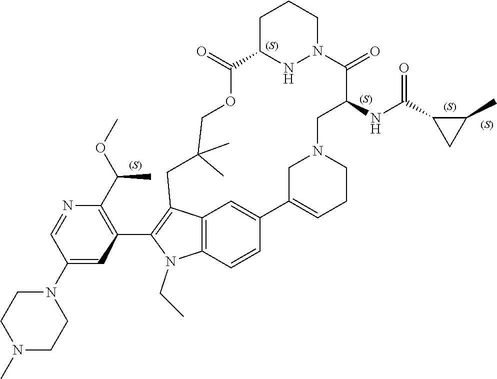 |
| DA 161 | 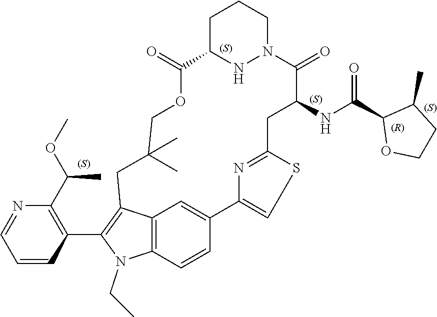 | DA 338 | 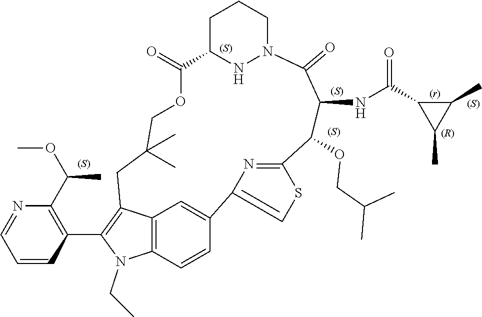 |
| DA 162 | 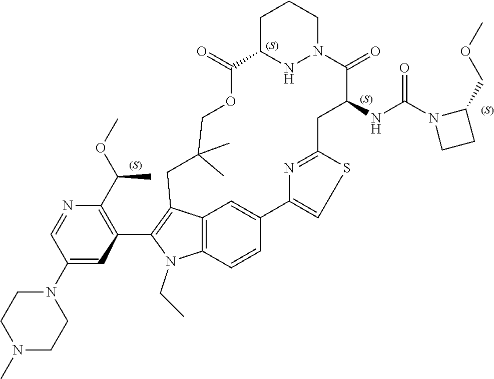 | DA 339 | 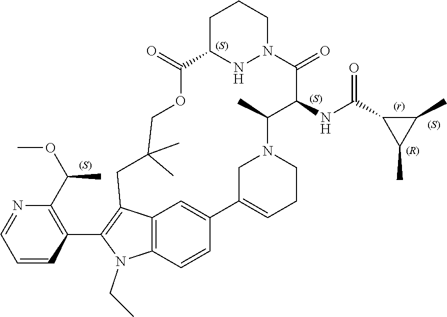 |
| DA 163 | 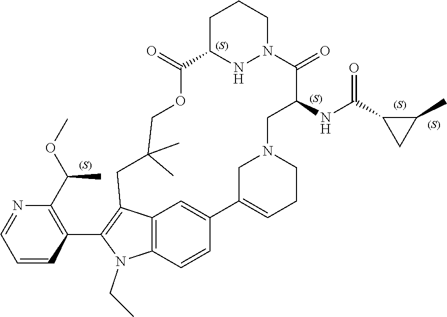 | DA 340 | 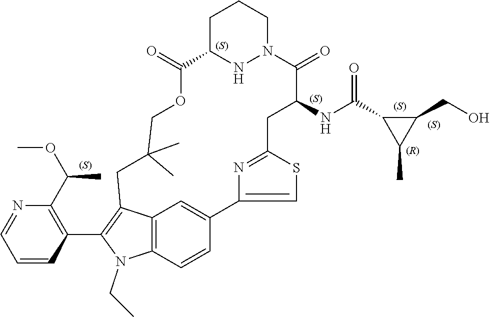 |
| DA 164 | 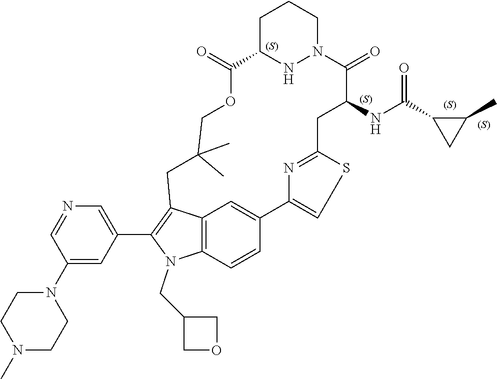 | DA 341 | 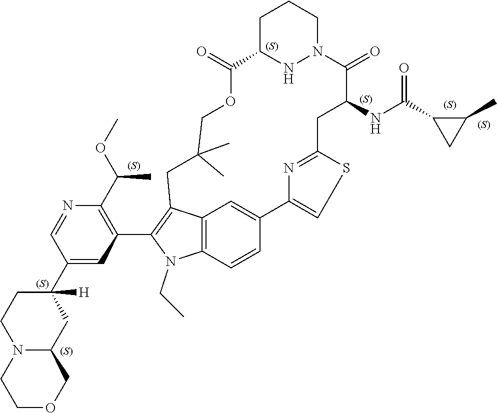 |
| DA 165 | 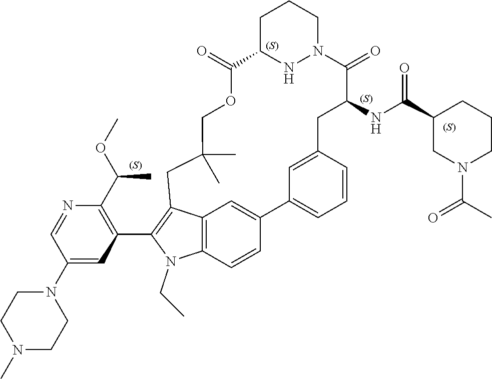 | DA 342 | 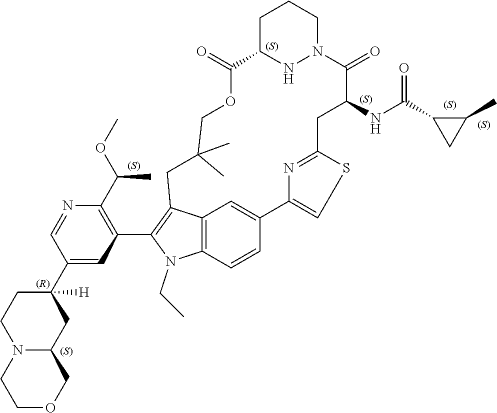 |
| DA 166 | 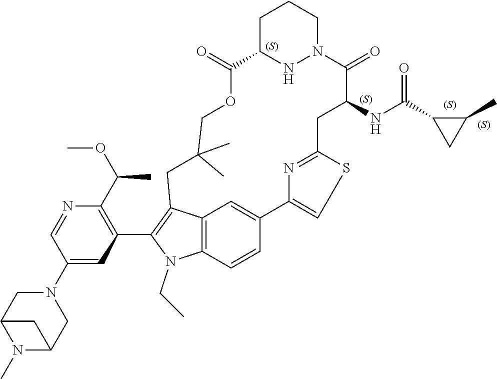 | DA 343 | 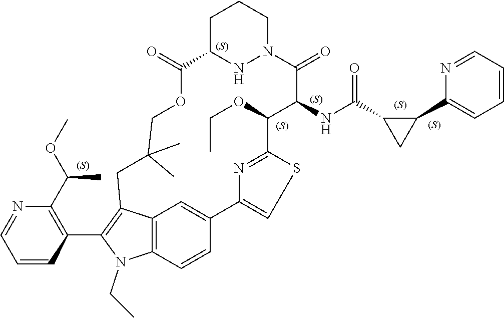 |
| DA 167 | 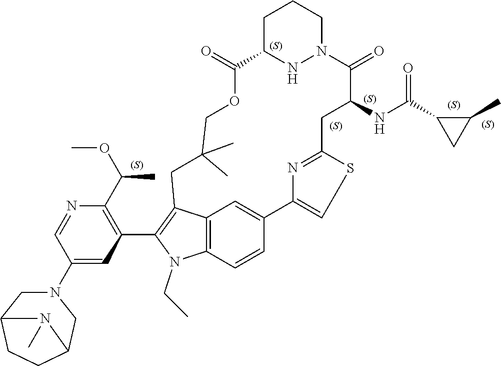 | DA 344 | 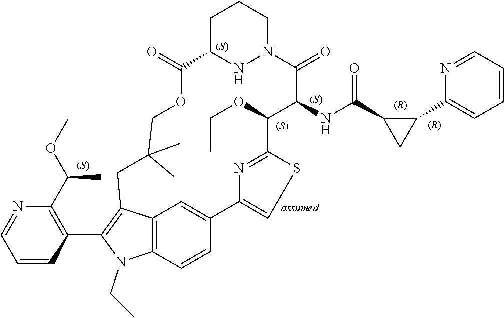 |
| DA 168 | 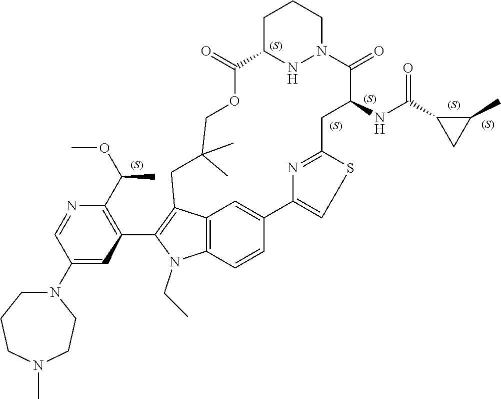 | DA 345 | 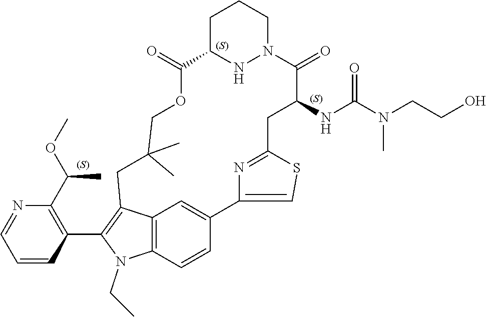 |
| DA 169 | 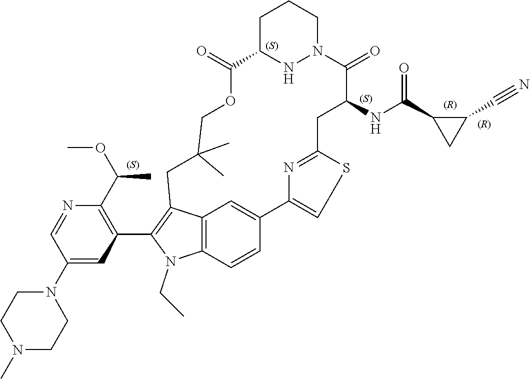 | DA 346 | 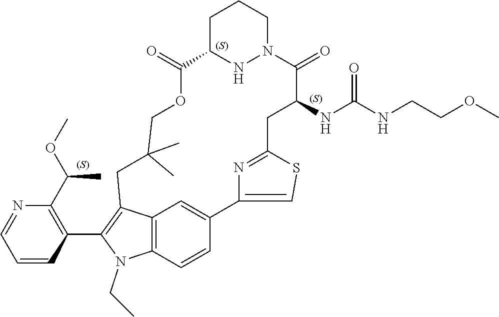 |
| DA 170 | 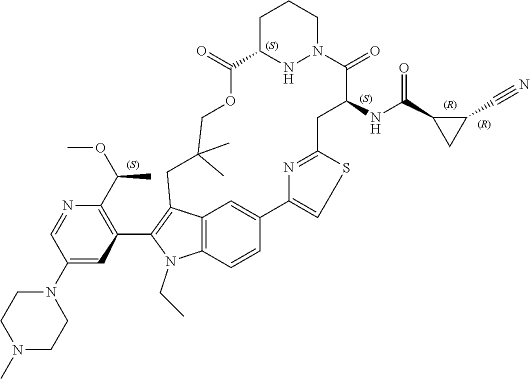 | DA 347 | 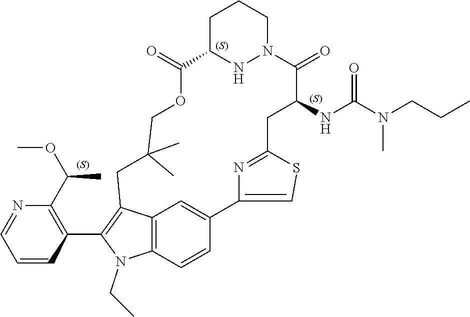 |
| DA 171 | 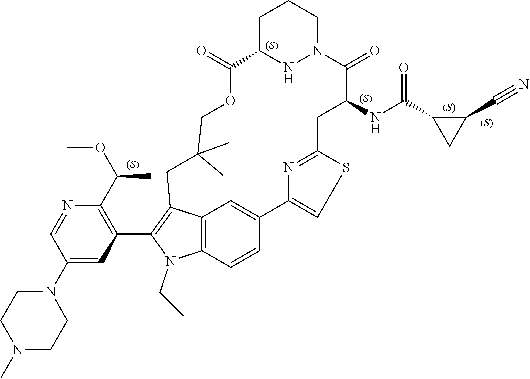 | DA 348 | 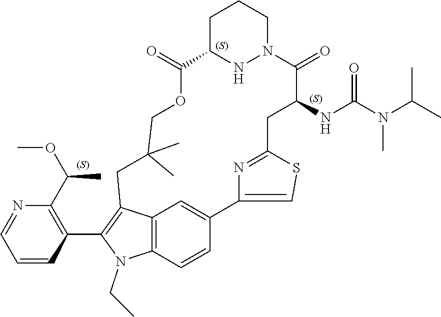 |
| DA 172 | 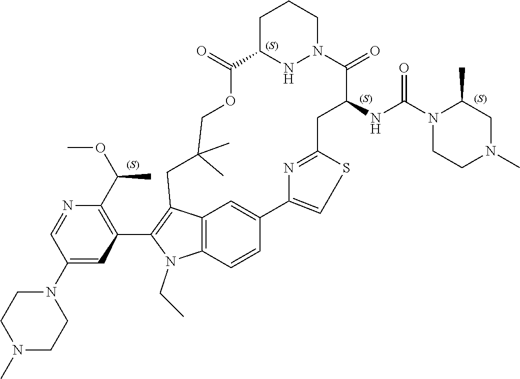 | DA 349 | 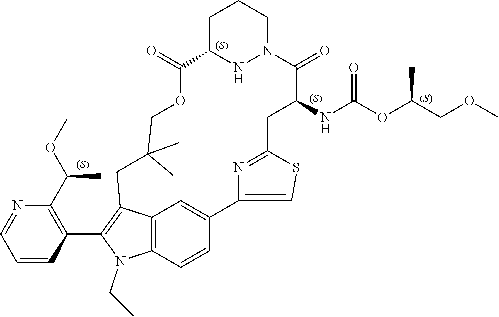 |
| DA 173 | 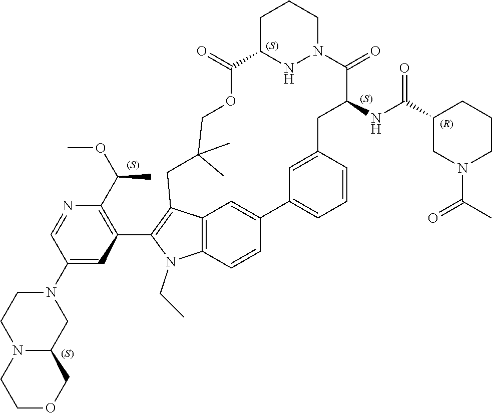 | DA 350 | 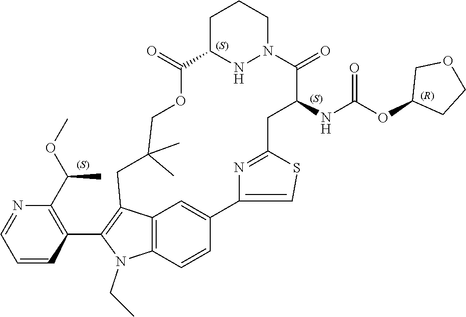 |
| DA 174 | 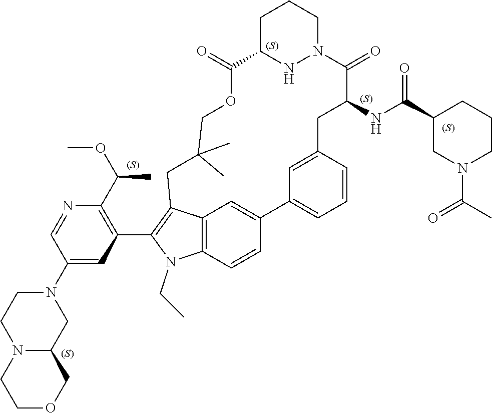 | DA 351 | 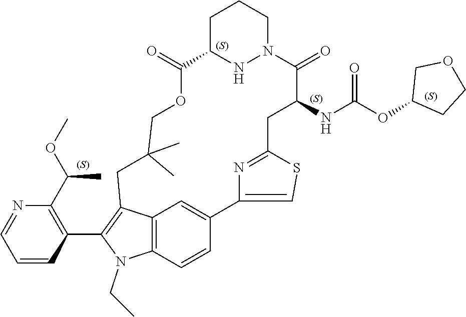 |
| DA 175 | 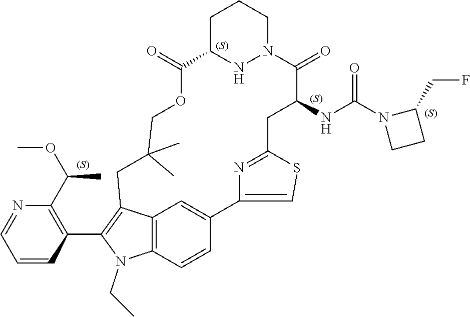 | DA 352 | 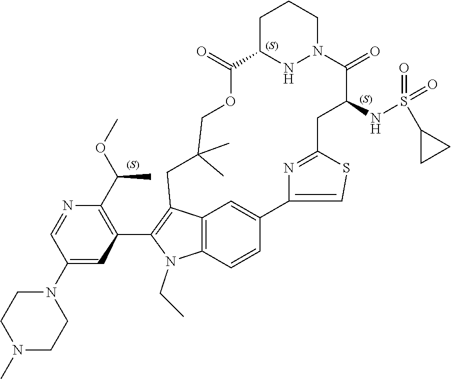 |
| DA 176 | 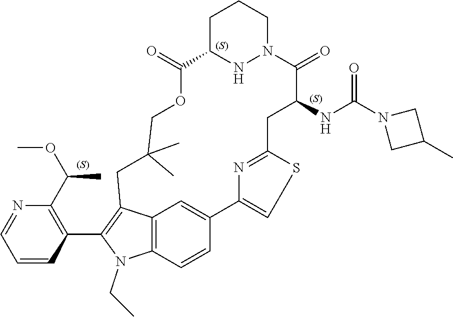 | | |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereochemistry of such diastereomer may not be known.

In some embodiments, the RAS(ON) inhibitor is selected from Table D1 b, or a pharmaceutically acceptable salt or stereoisomer thereof. In some embodiments, the RAS(ON) inhibitor is selected from Table D1 b, or a pharmaceutically acceptable salt or atropisomer thereof.

TABLE D1b
Certain Compounds of the Present Invention

| Ex. | |
|---|---|
| # | Structure |
| DA 354 | 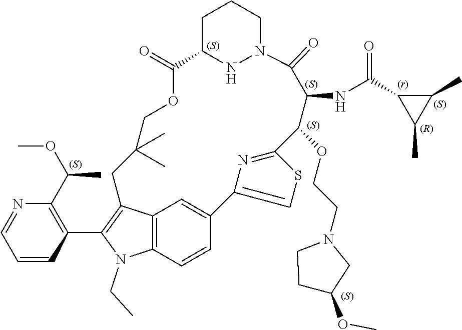 |
| DA 355 | 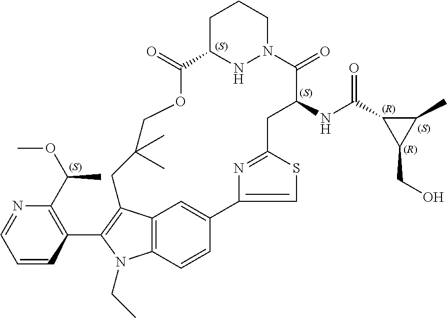 |
| DA 356 | 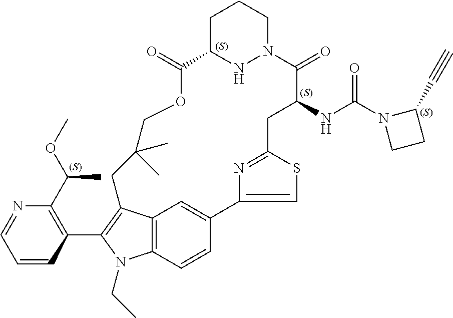 |
| DA 357 | 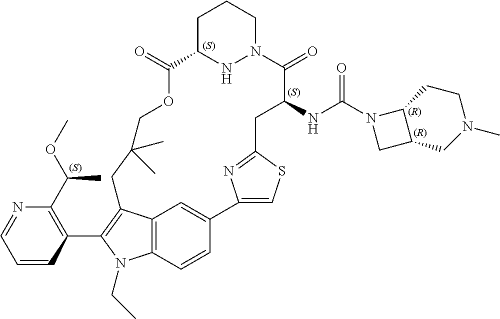 |
| DA 358 | 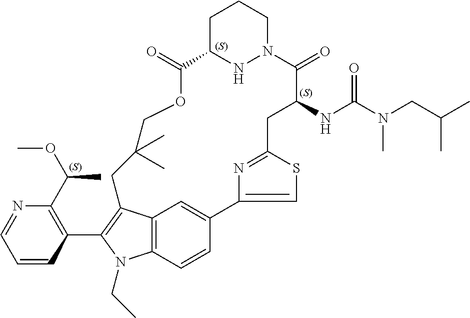 |
| DA 359 | 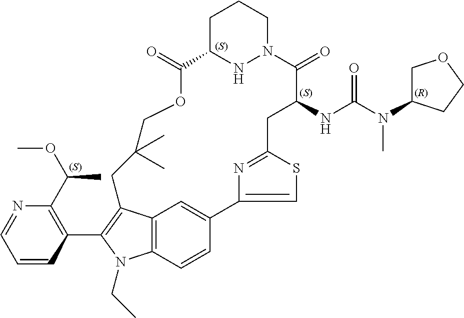 |
| DA 360 | 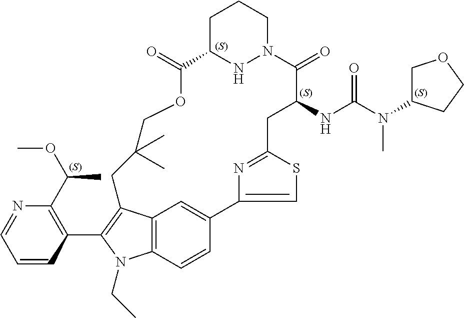 |
| DA 361 | 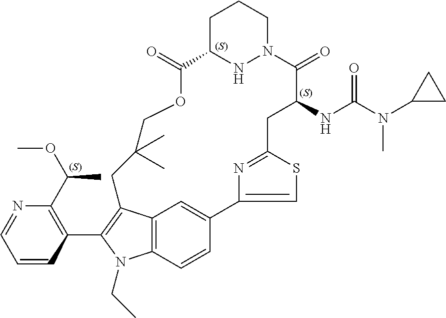 |
| DA 362 | 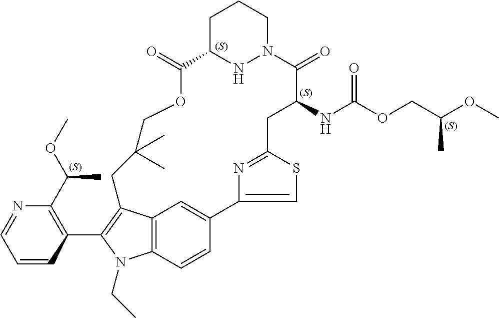 |
| DA 363 | 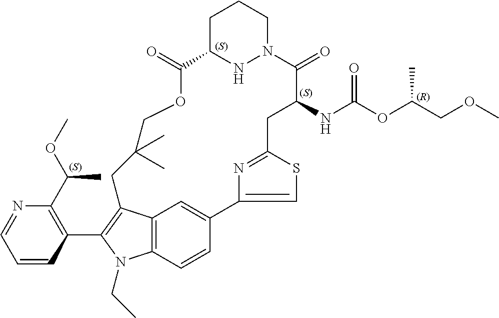 |
| DA 364 | 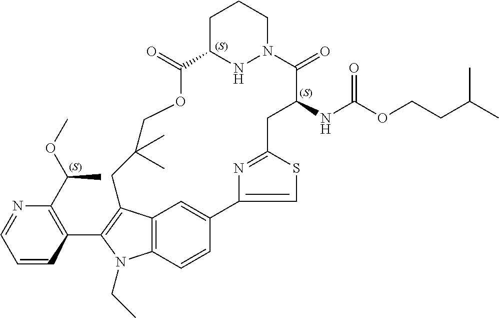 |
| DA 365 | 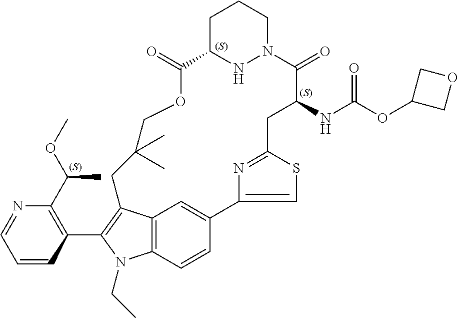 |
| DA 366 | 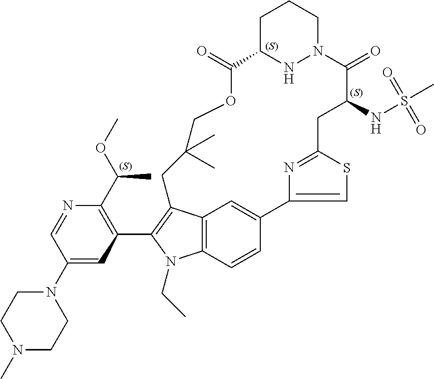 |
| DA 367 | 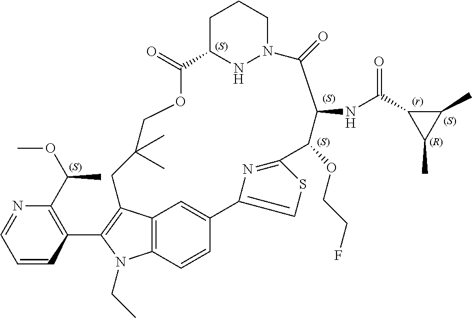 |
| DA 368 | 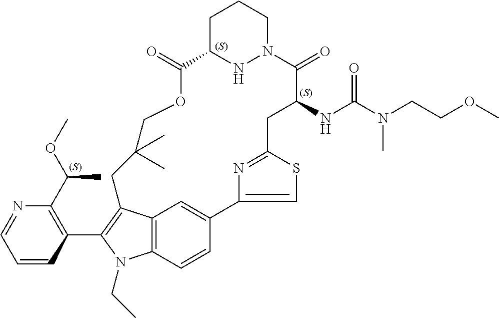 |
| DA 369 | 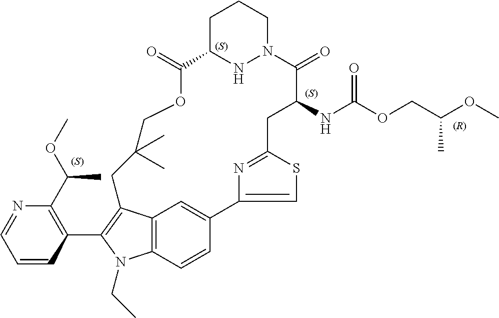 |
| DA 370 | 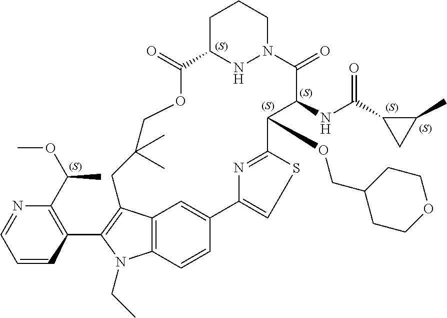 |
| DA 371 | 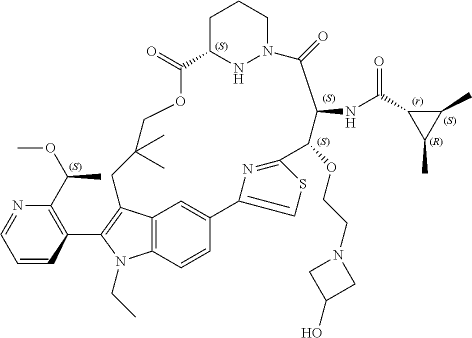 |
| DA 372 | 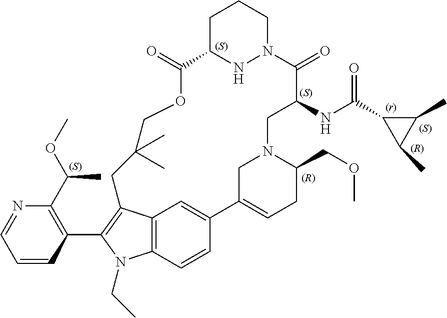 |
| DA 373 | 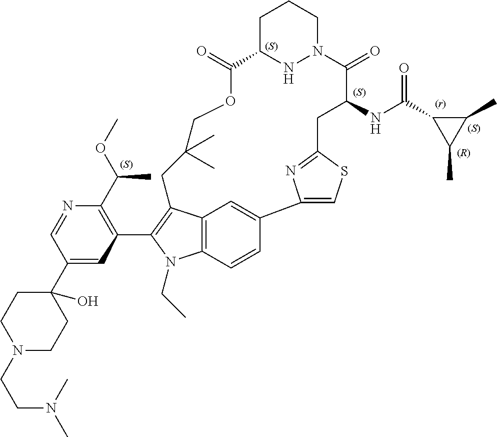 |
| DA 374 | 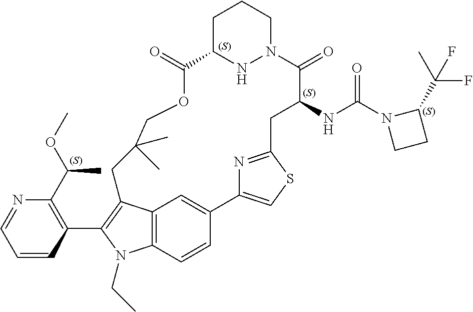 |
| DA 375 | 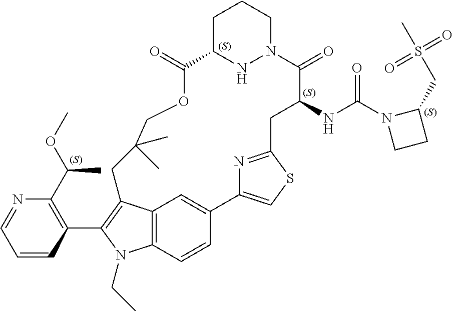 |
| DA 376 | 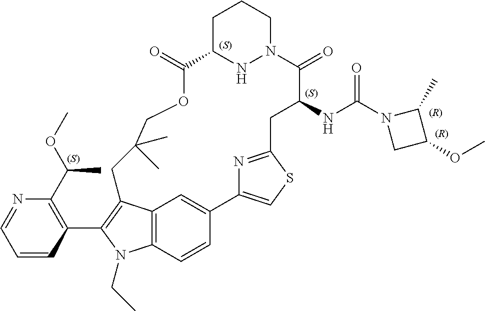 |
| DA 377 | 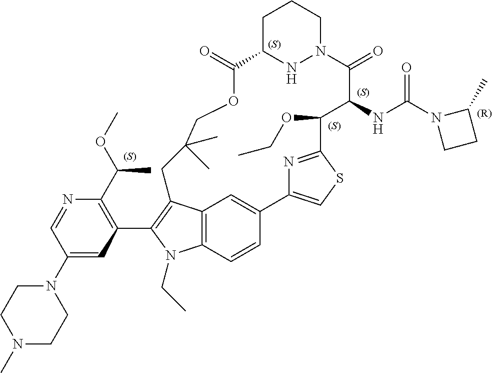 |
| DA 378 | 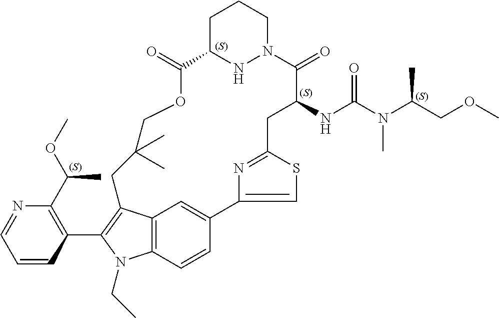 |
| DA 379 | 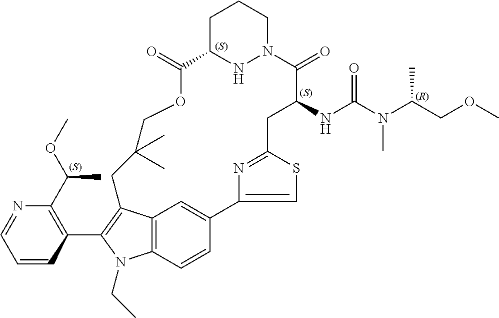 |
| DA 380 | 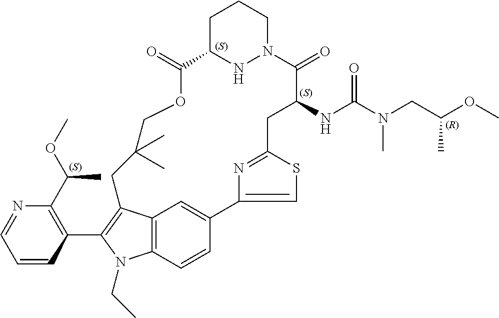 |
| DA 381 | 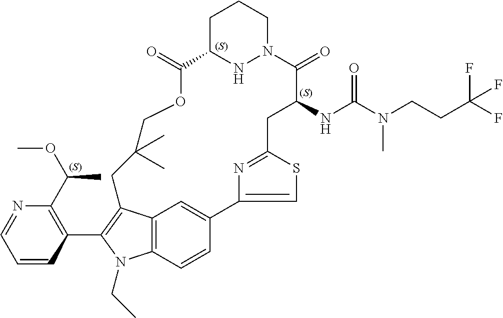 |
| DA 382 | 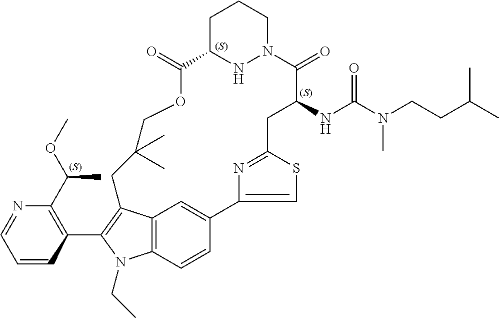 |
| DA 383 | 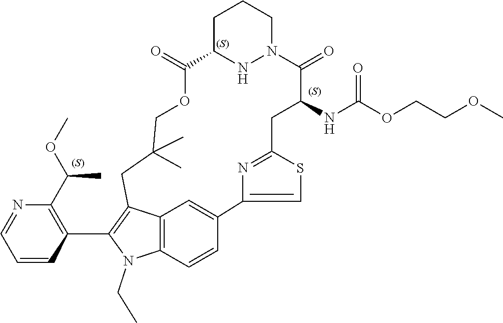 |
| DA 384 | 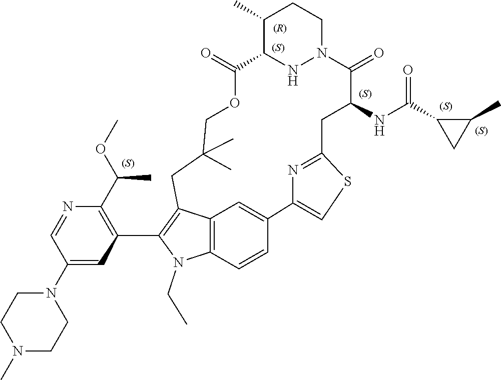 |
| DA 385 | 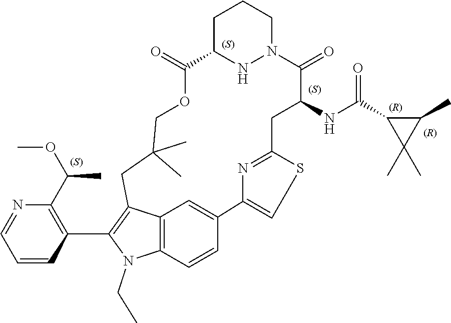 |
| DA 386 | 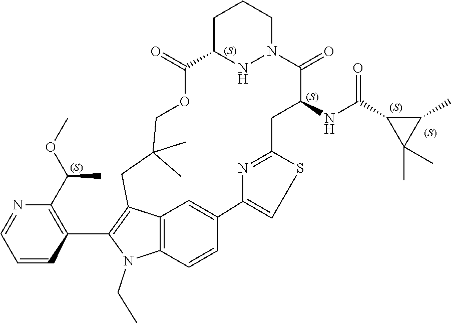 |
| DA 387 | 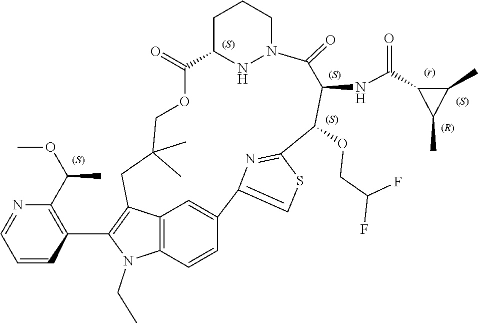 |
| DA 388 | 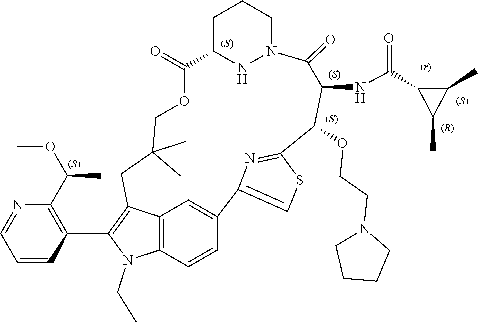 |
| DA 389 | 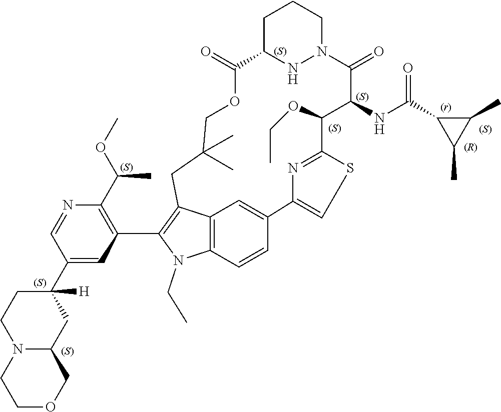 |
| DA 390 | 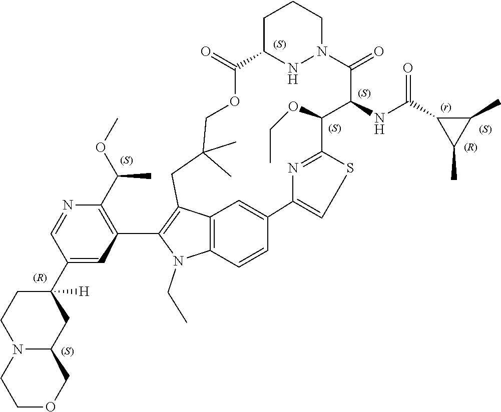 |
| DA 391 | 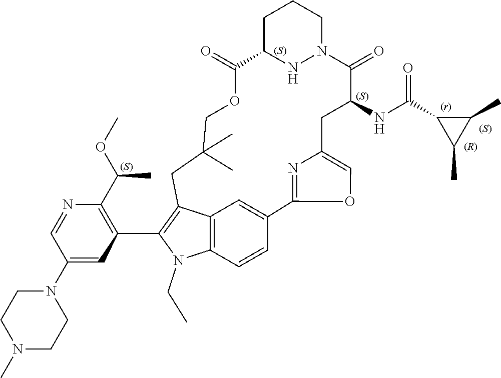 |
| DA 392 | 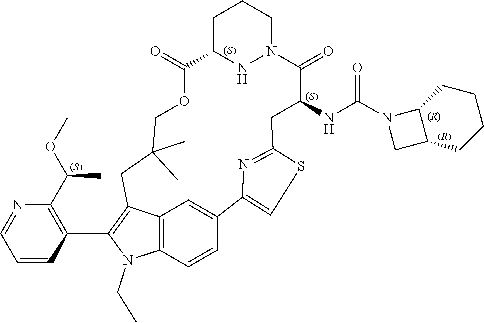 |
| DA 393 | 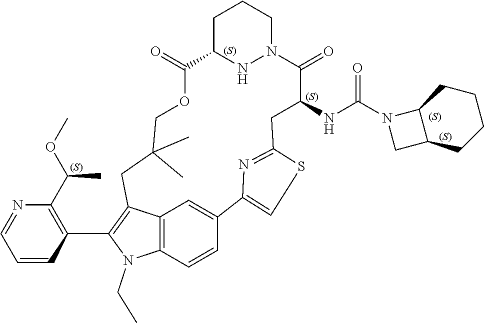 |
| DA 394 | 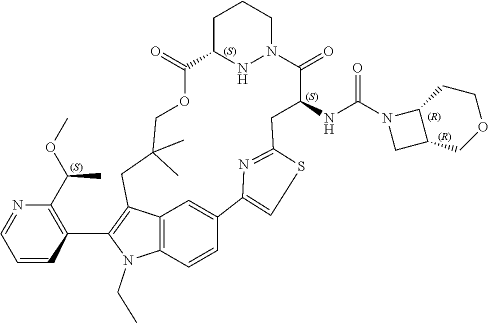 |
| DA 395 | 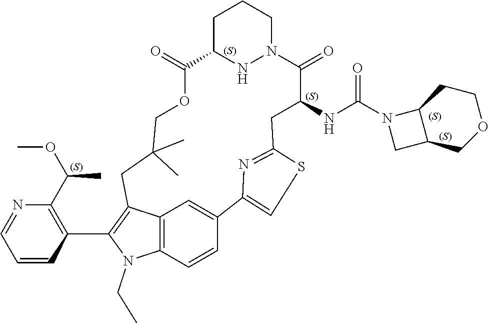 |
| DA 396 | 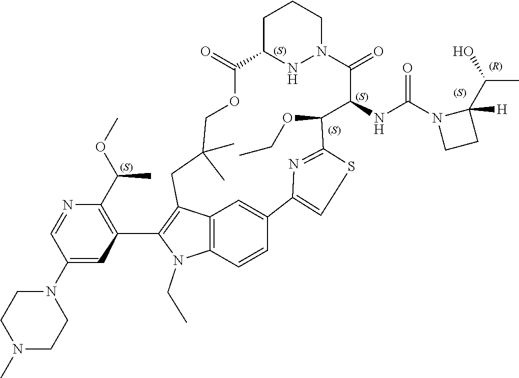 |
| DA 397 | 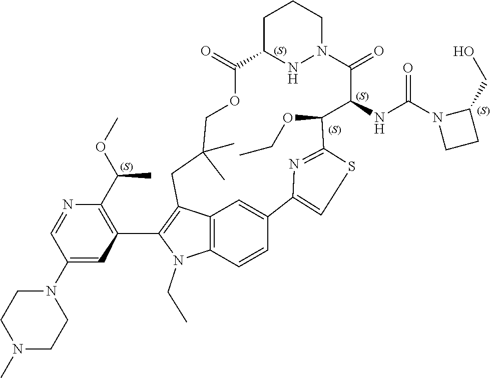 |
| DA 398 | 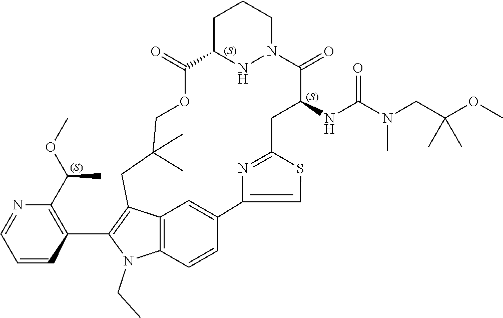 |
| DA 399 | 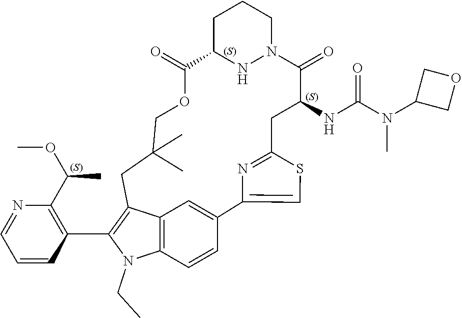 |
| DA 400 | 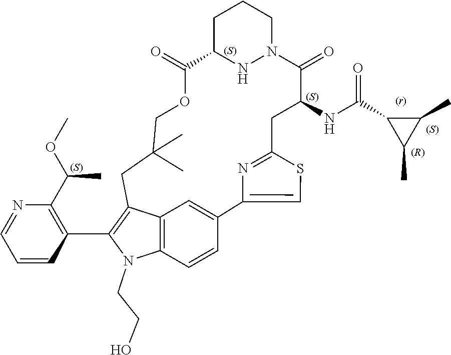 |
| DA 401 | 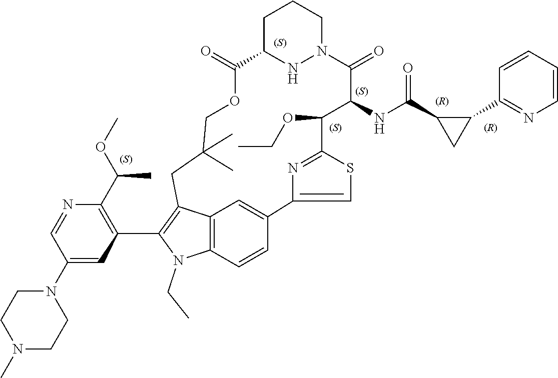 |
| DA 402 | 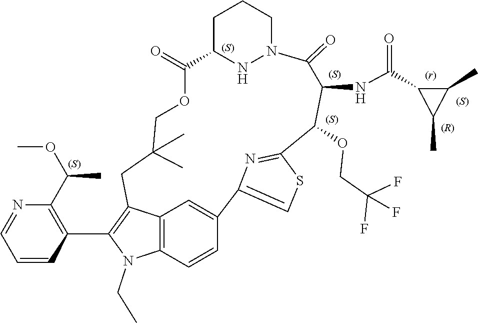 |
| DA 403 | 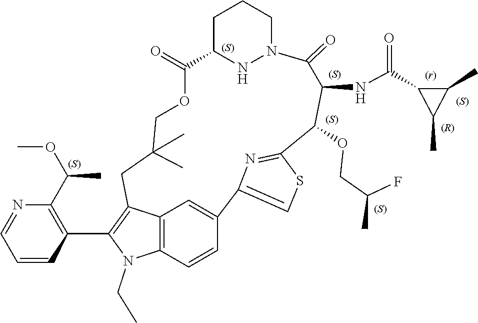 |
| DA 404 | 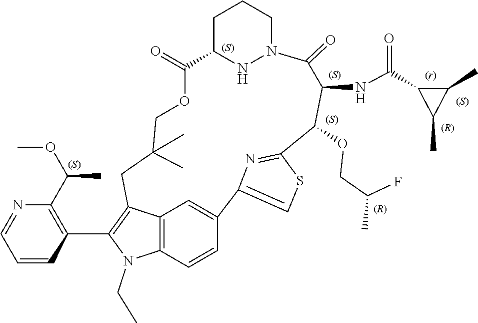 |
| DA 405 | 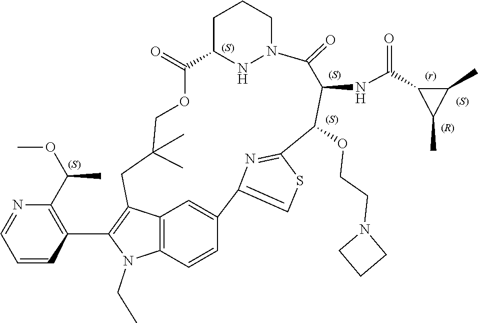 |
| DA 406 | 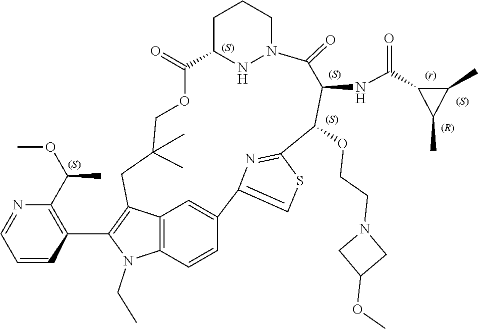 |
| DA 407 | 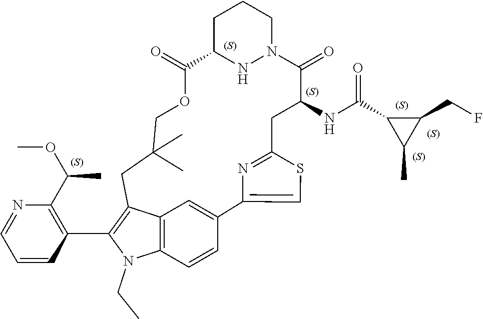 |
| DA 408 | 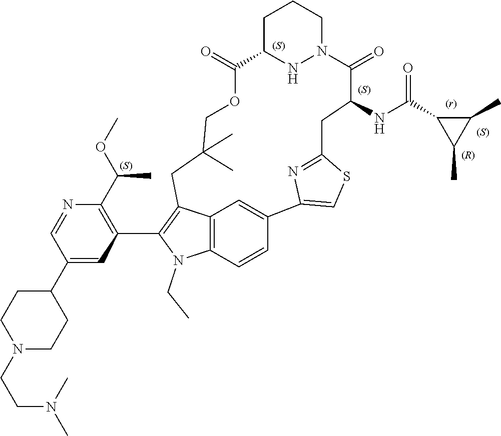 |
| DA 409 | 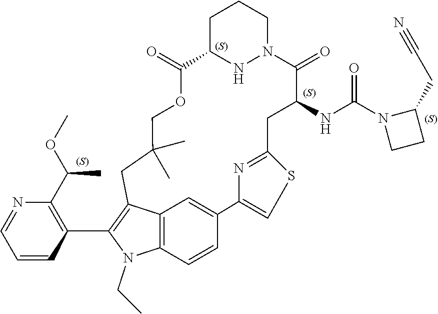 |
| DA 410 | 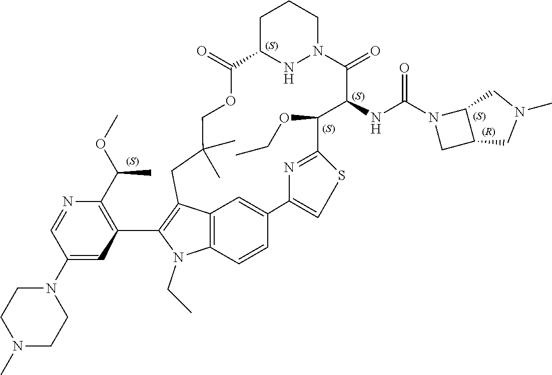 |
| DA 411 | 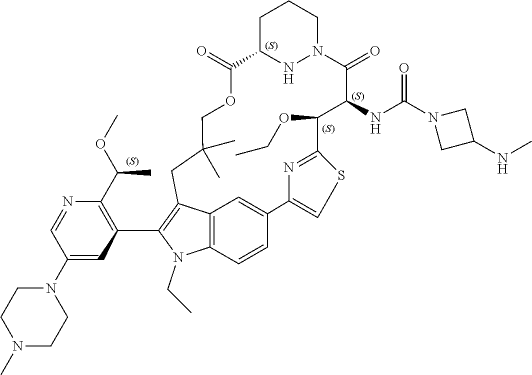 |
| DA 412 | 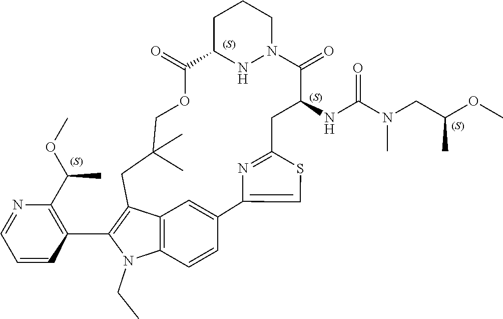 |
| DA 413 | 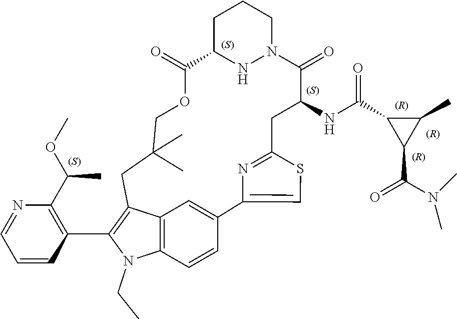 |
| DA 414 | 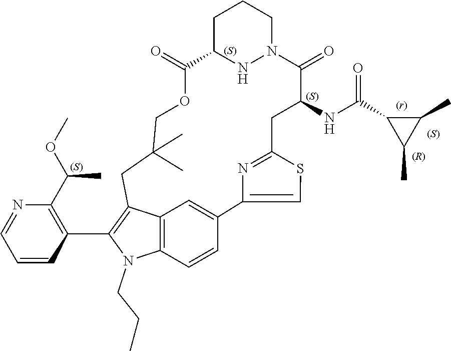 |
| DA 415 | 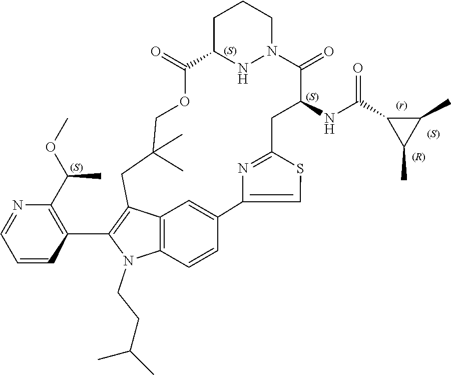 |
| DA 416 | 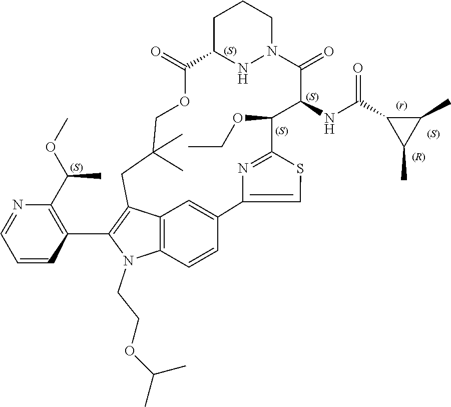 |
| DA 417 | 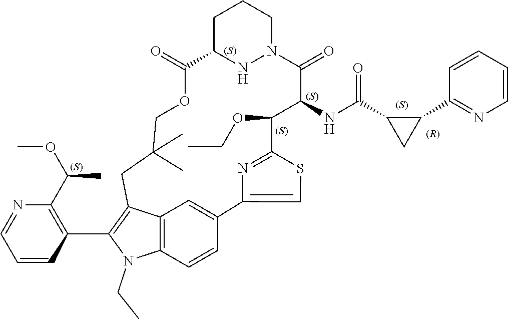 |
| DA 418 | 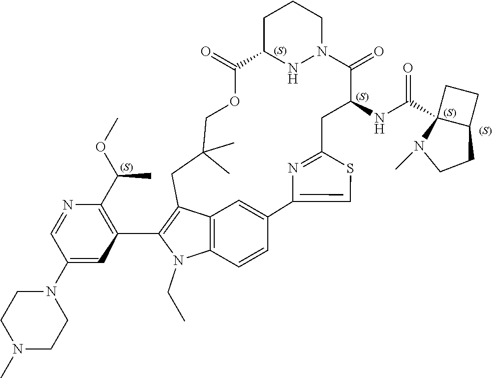 |
| DA 419 | 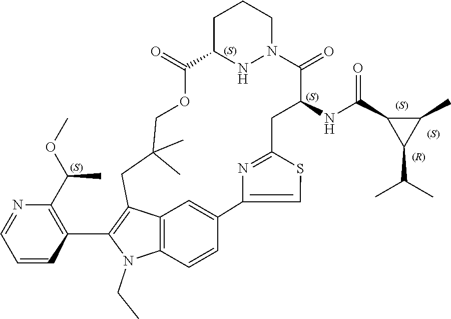 |
| DA 420 | 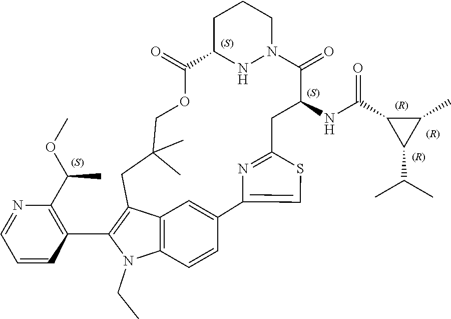 |
| DA 421 | 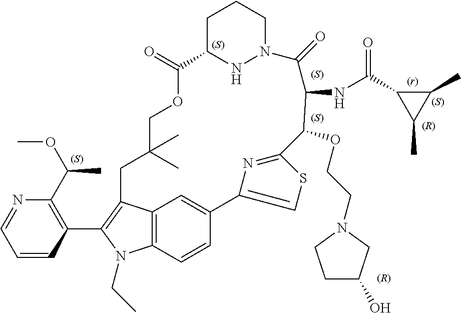 |
| DA 422 | 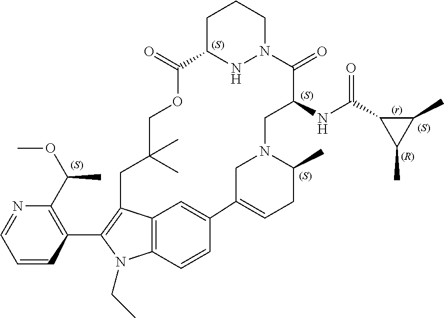 |
| DA 423 | 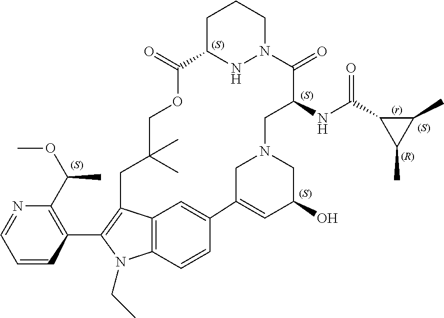 |
| DA 424 | 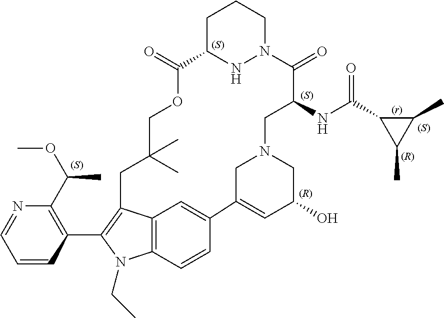 |
| DA 425 | 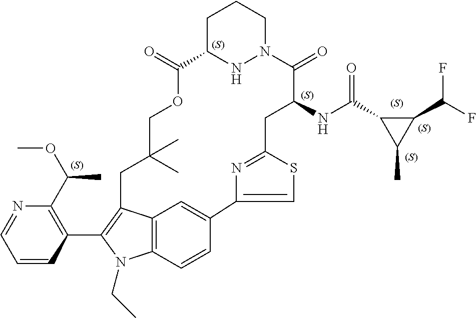 |
| DA 426 | 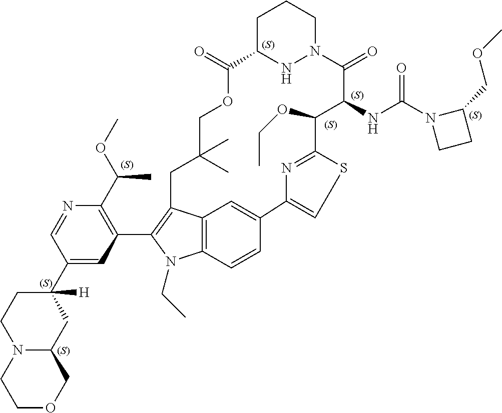 |
| DA 427 | 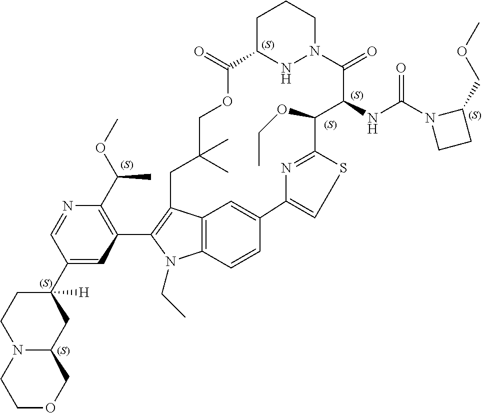 |
| DA 428 | 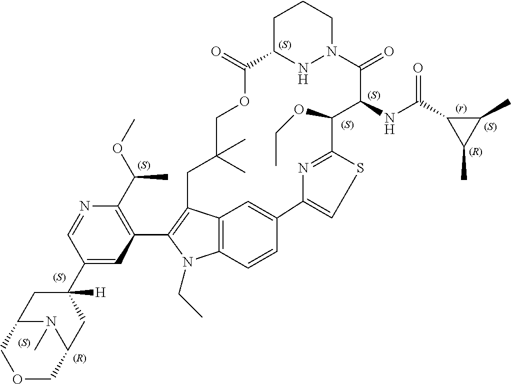 |
| DA 429 | 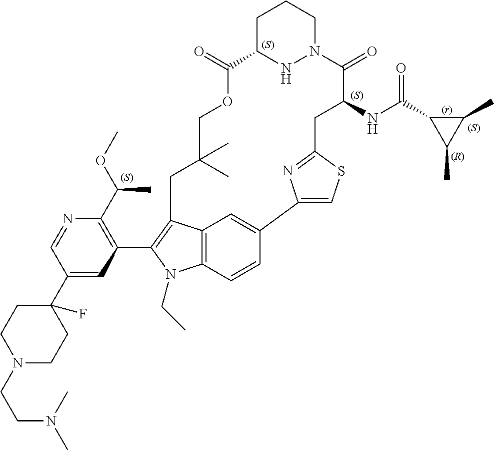 |
| DA 430 | 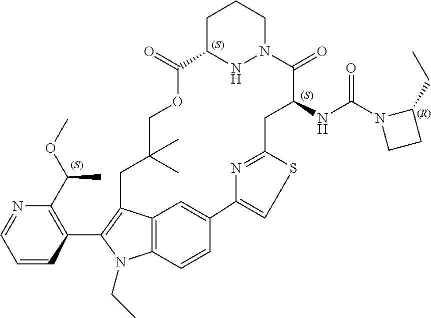 |
| DA 431 | 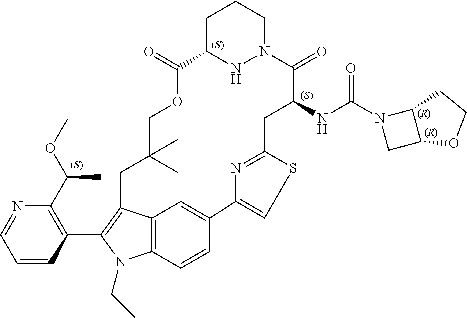 |
| DA 432 | 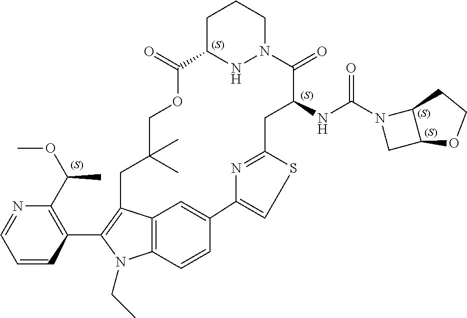 |
| DA 433 | 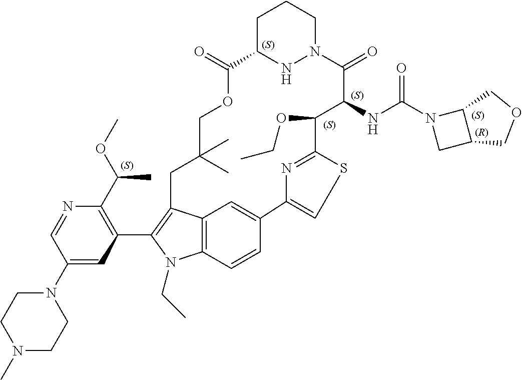 |
| DA 434 | 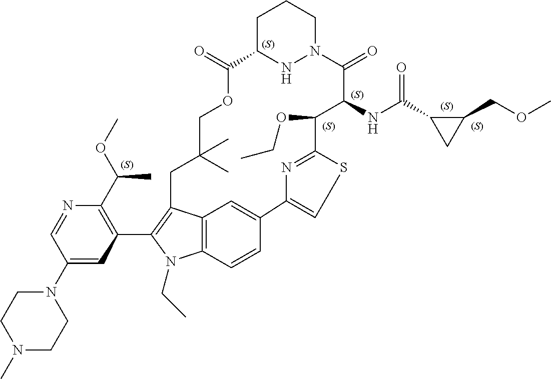 |
| DA 435 | 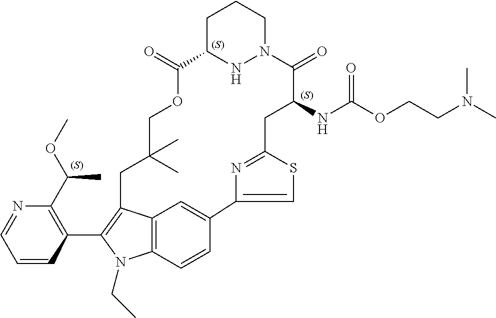 |
| DA 436 | 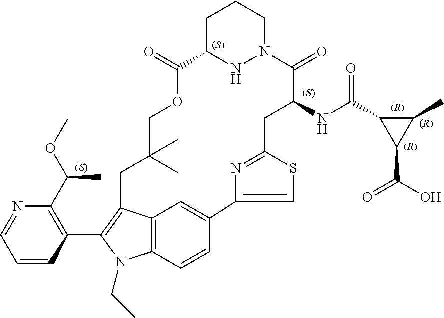 |
| DA 437 | 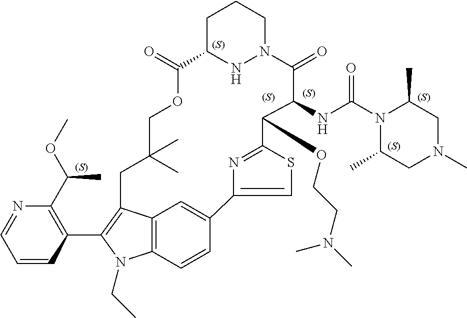 |
| DA 438 | 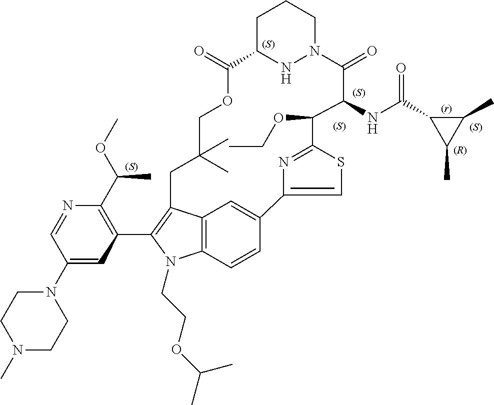 |
| DA 439 | 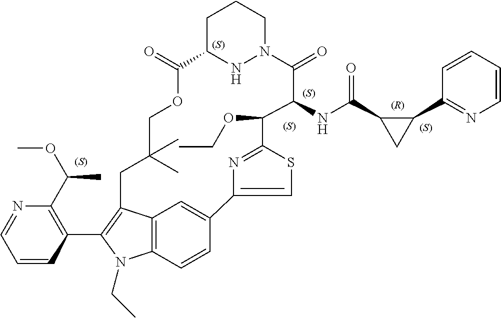 |
| DA 440 | 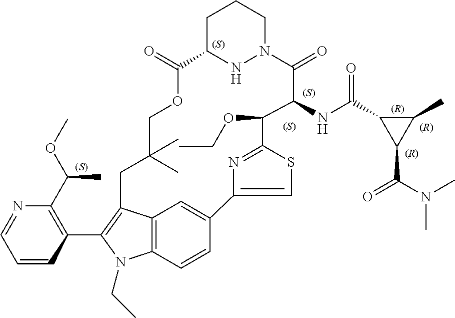 |
| DA 441 | 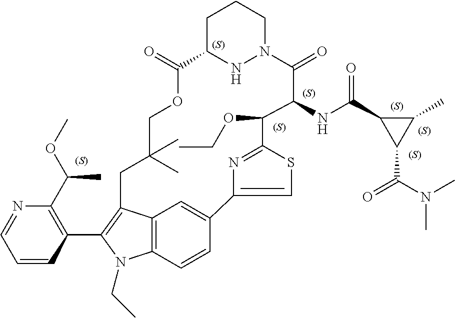 |
| DA 442 | 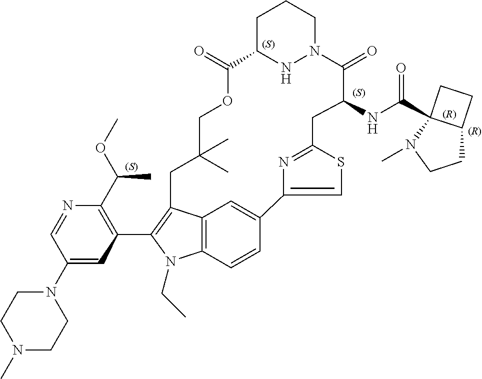 |
| DA 443 | 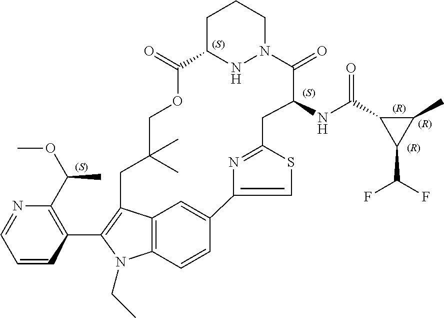 |
| DA 444 | 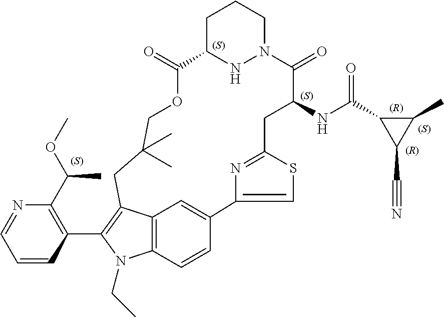 |
| DA 445 | 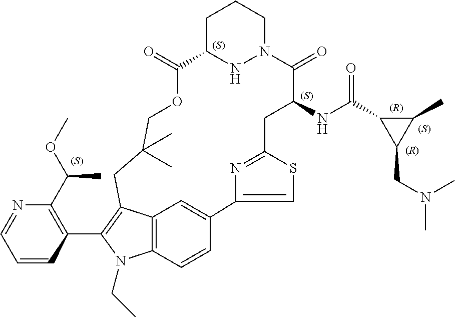 |
| DA 446 | 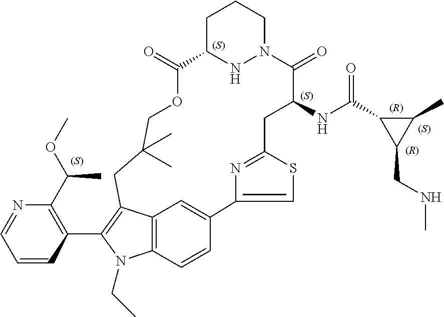 |
| DA 447 | 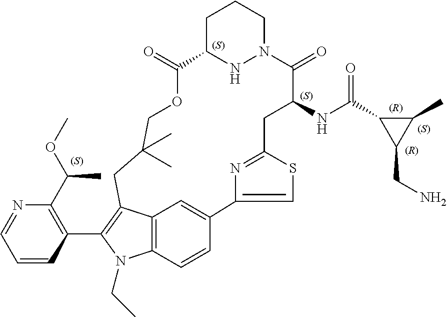 |
| DA 448 | 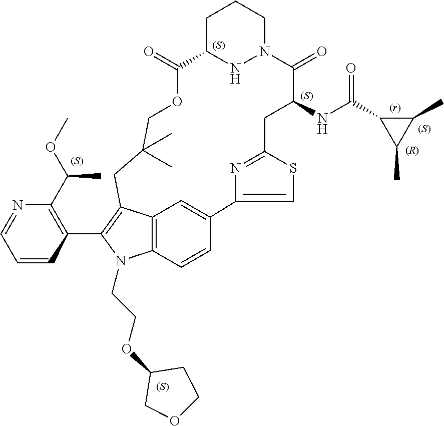 |
| DA 449 | 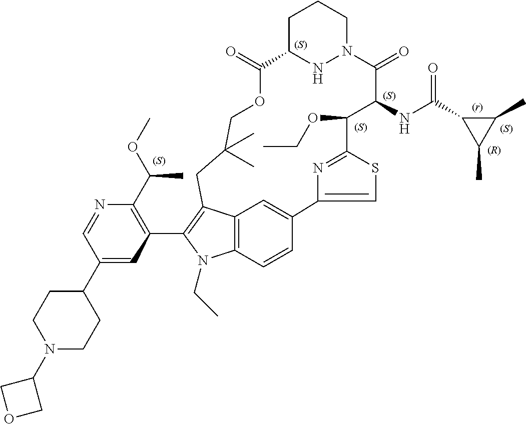 |
| DA 450 | 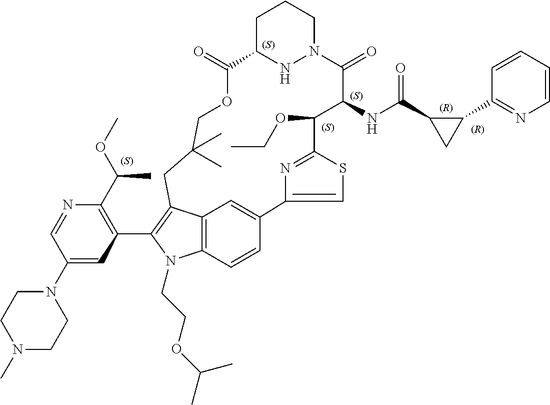 |
| DA 451 | 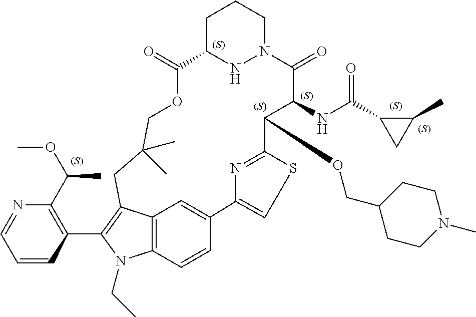 |
| DA 452 | 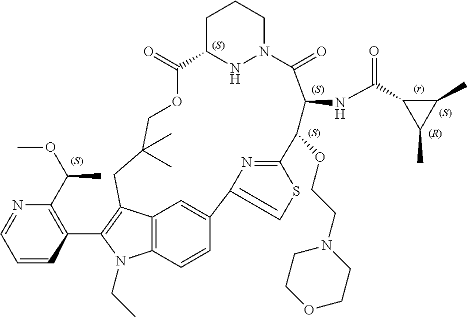 |
| DA 453 | 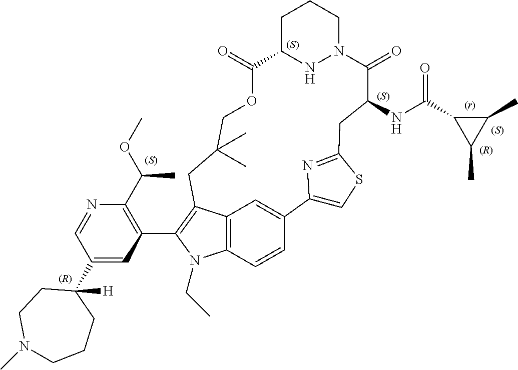 |
| DA 454 | 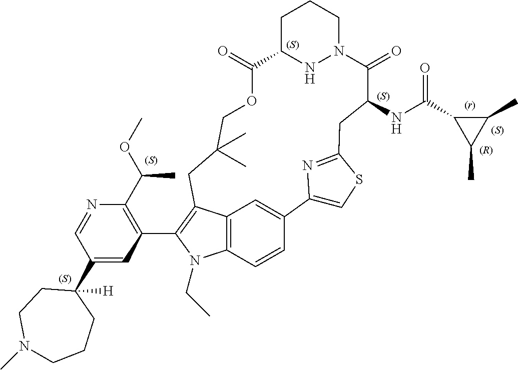 |
| DA 455 | 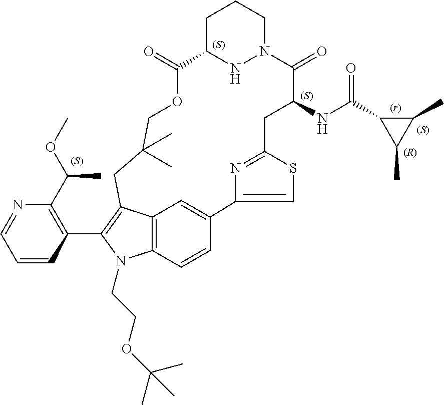 |
| DA 456 | 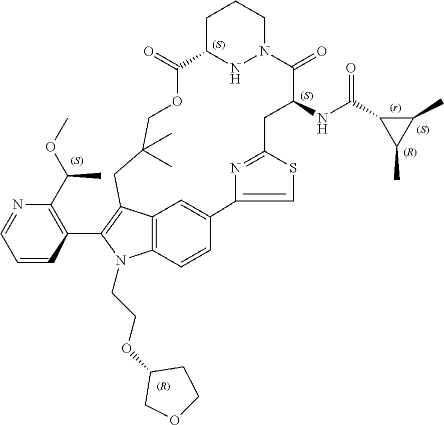 |
| DA 457 | 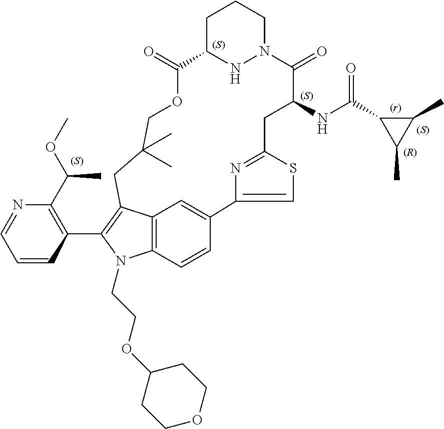 |
| DA 458 | 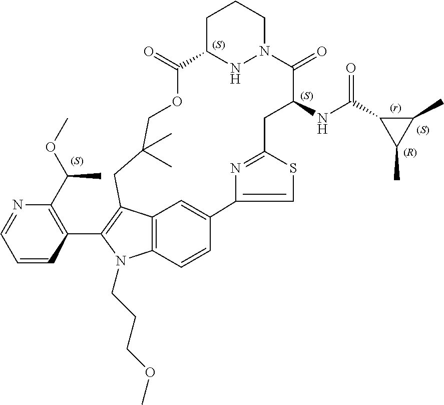 |
| DA 459 | 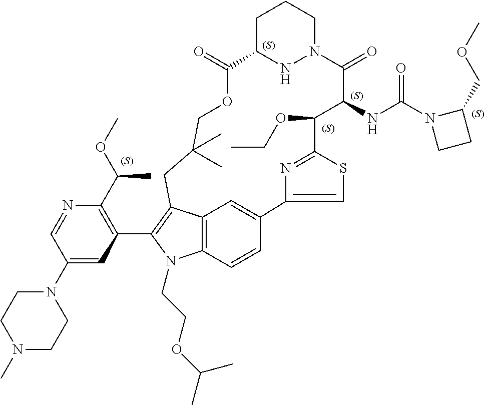 |
| DA 460 | 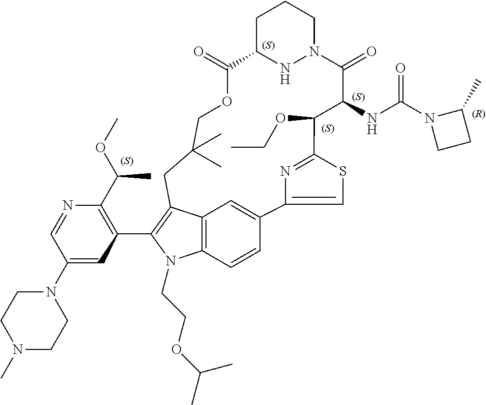 |
| DA 461 | 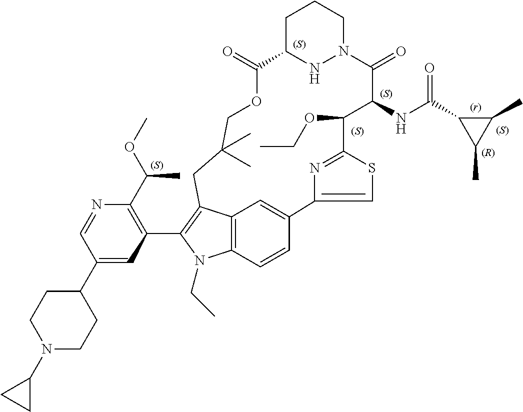 |
| DA 462 | 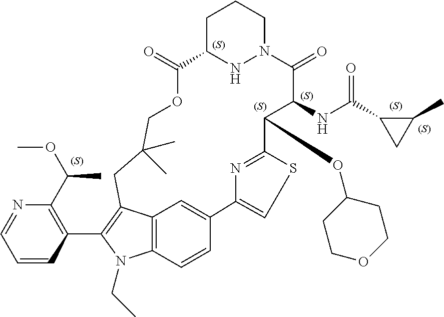 |
| DA 463 | 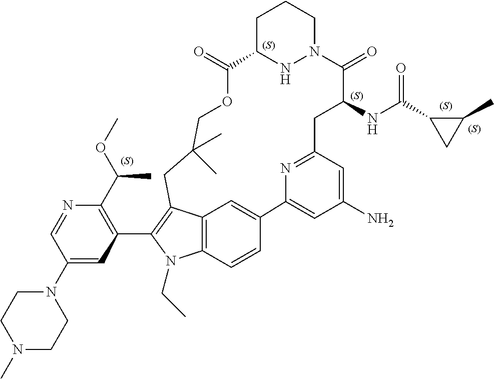 |
| DA 464 | 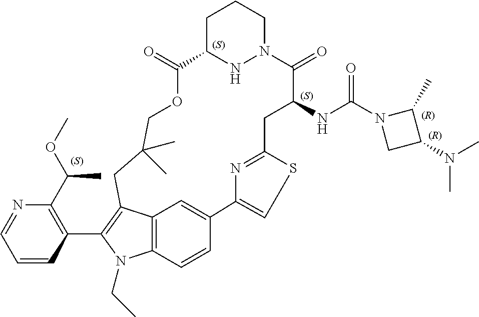 |
| DA 465 | 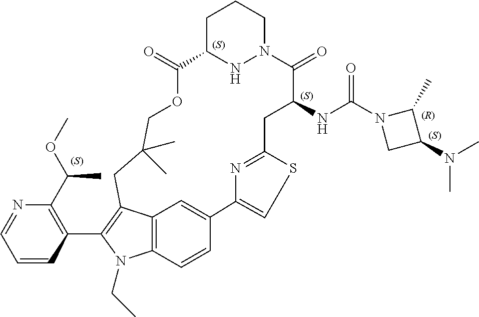 |
| DA 466 | 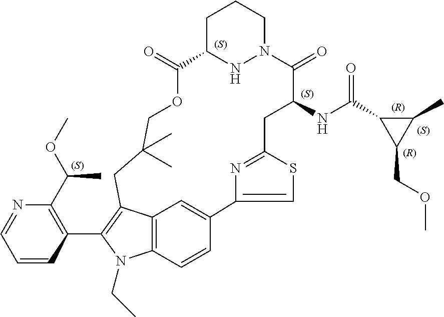 |
| DA 467 | 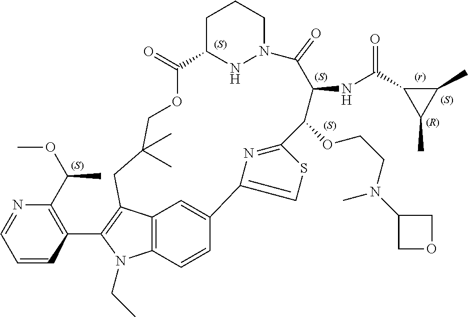 |
| DA 468 | 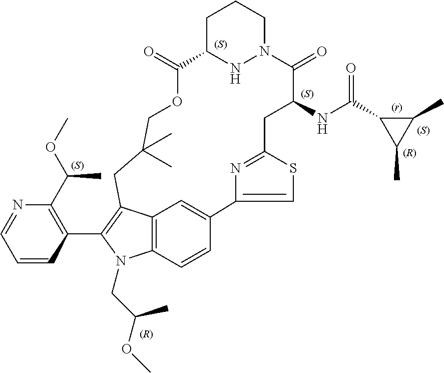 |
| DA 469 | 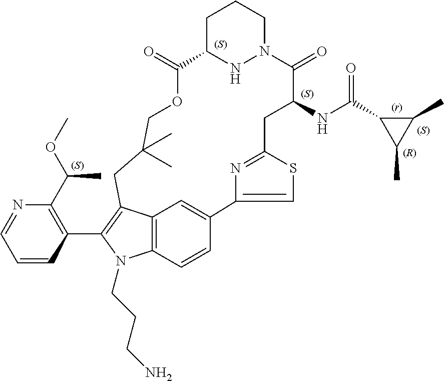 |
| DA 470 | 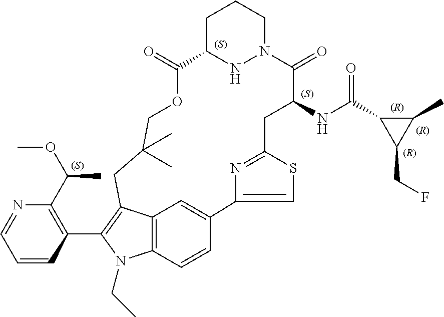 |
| DA 471 | 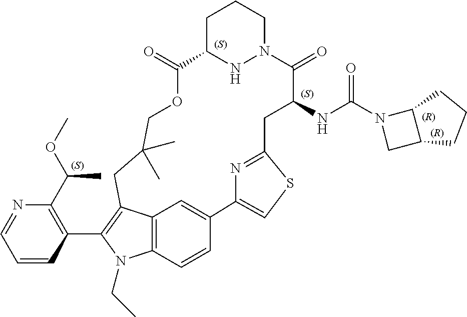 |
| DA 472 | 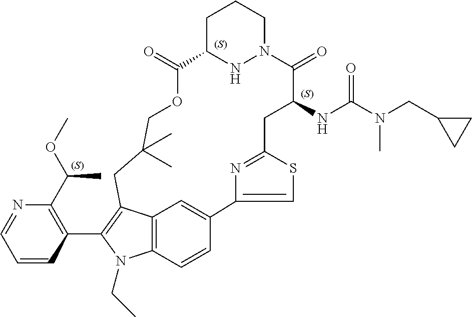 |
| DA 473 | 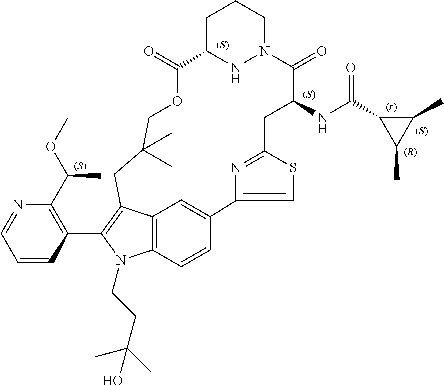 |
| DA 474 | 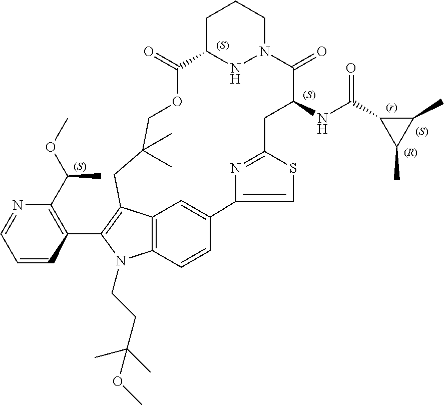 |
| DA 475 | 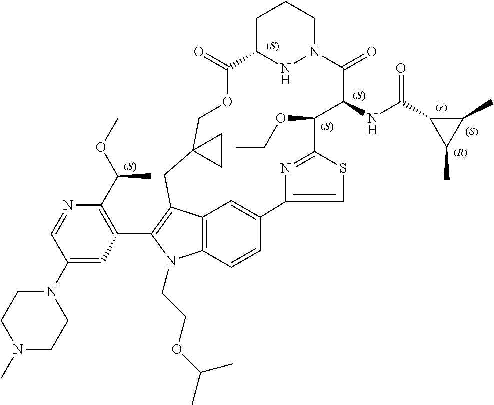 |
| DA 476 | 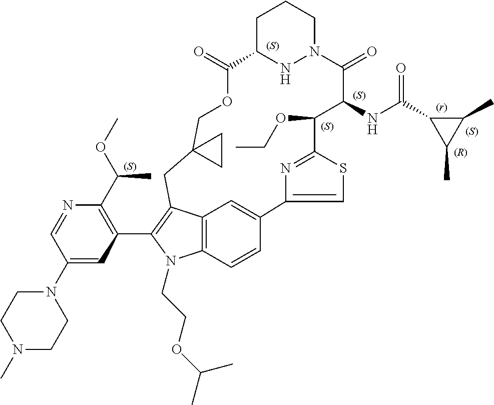 |
| DA 477 | 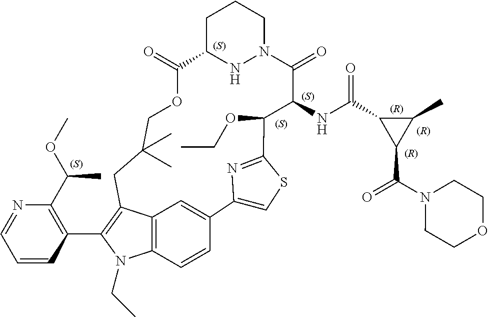 |
| DA 478 | 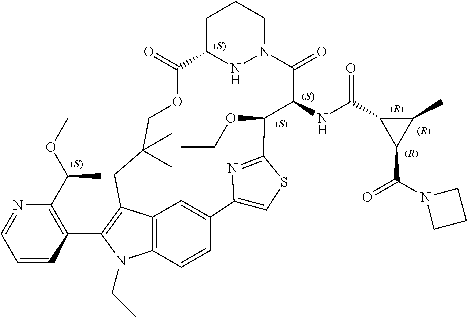 |
| DA 479 | 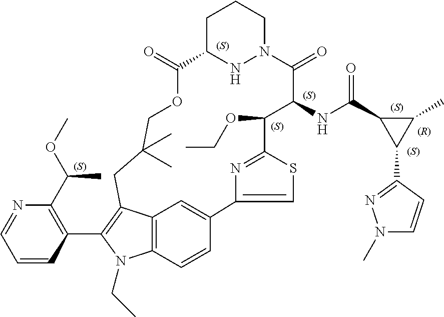 |
| DA 480 | 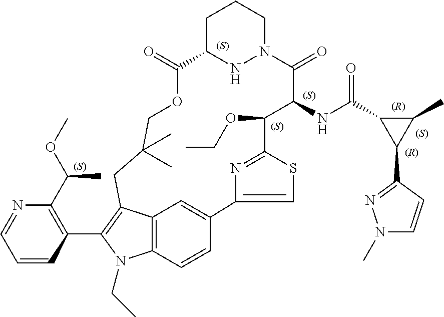 |
| DA 481 | 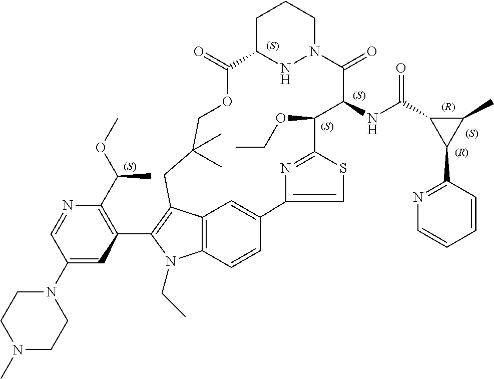 |
| DA 482 | 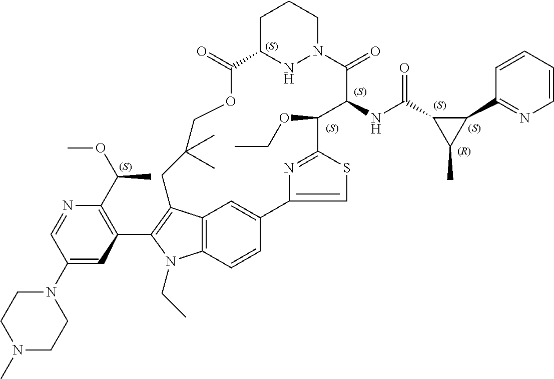 |
| DA 483 | 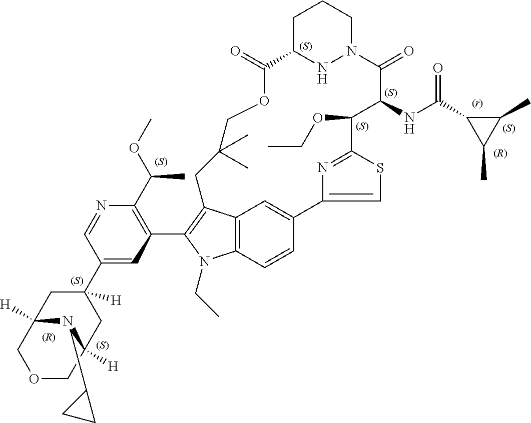 |
| DA 484 | 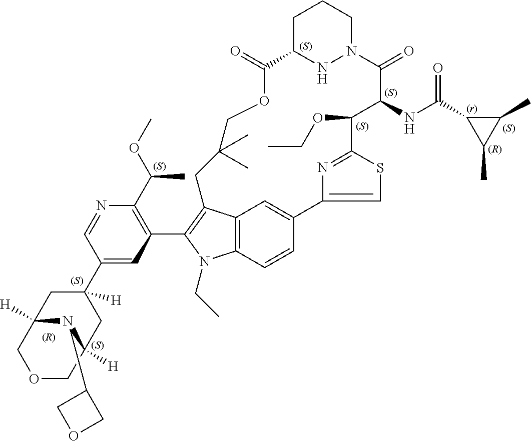 |
| DA 485 | 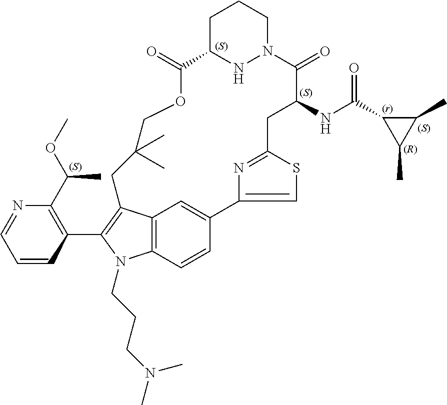 |
| DA 486 | 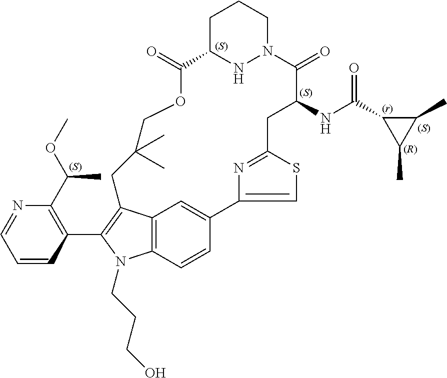 |
| DA 487 | 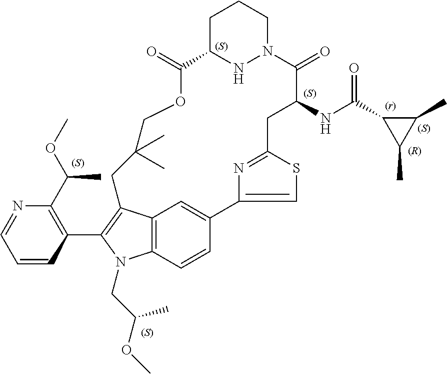 |
| DA 488 | 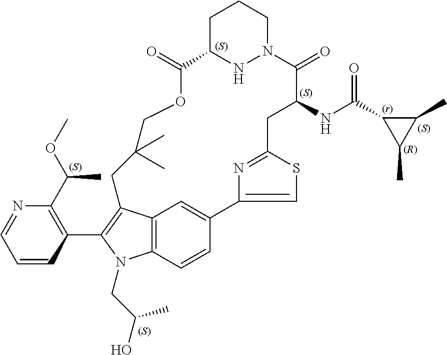 |
| DA 489 | 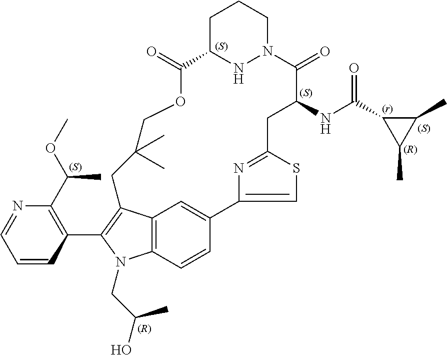 |
| DA 490 | 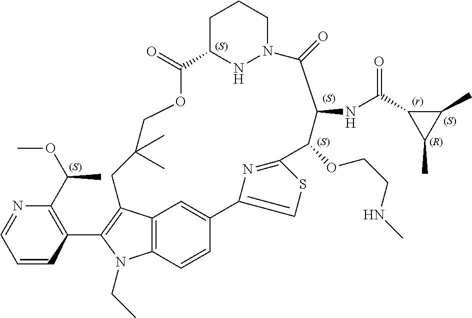 |
| DA 491 | 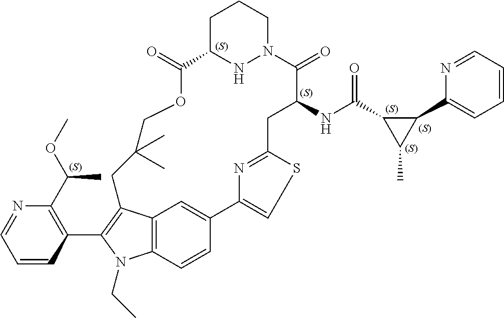 |
| DA 492 | 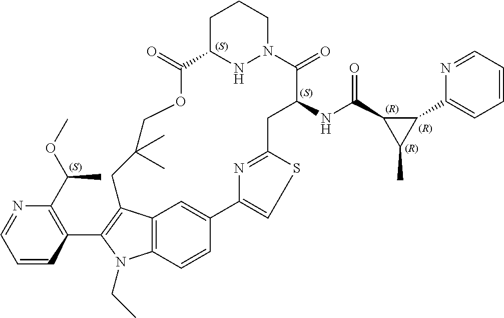 |
| DA 493 | 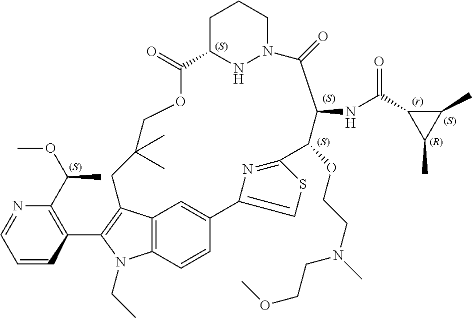 |
| DA 494 | 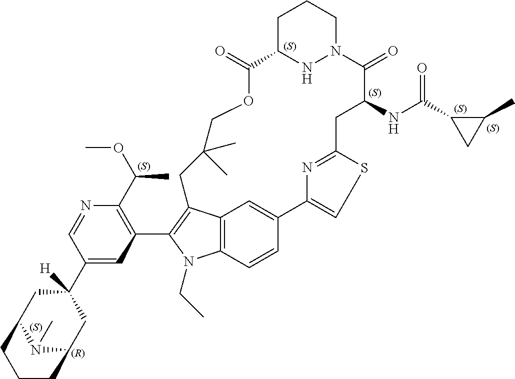 |
| DA 495 | 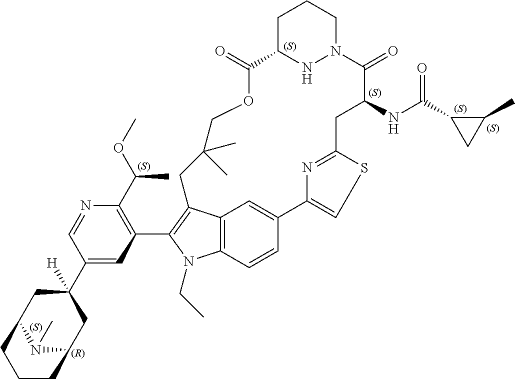 |
| DA 496 | 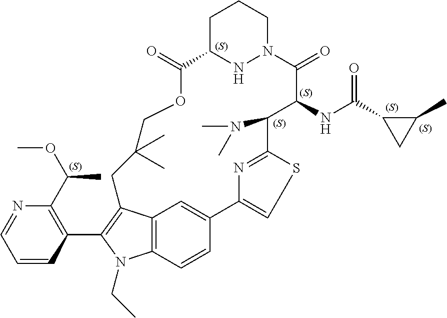 |
| DA 497 | 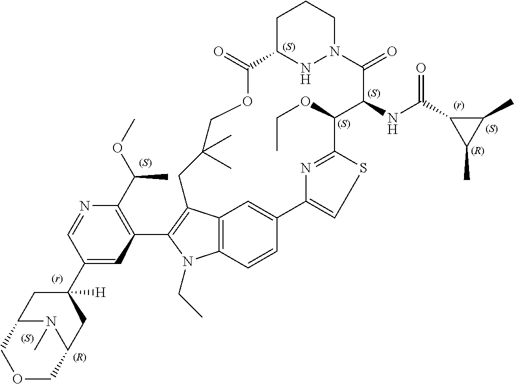 |
| DA 498 | 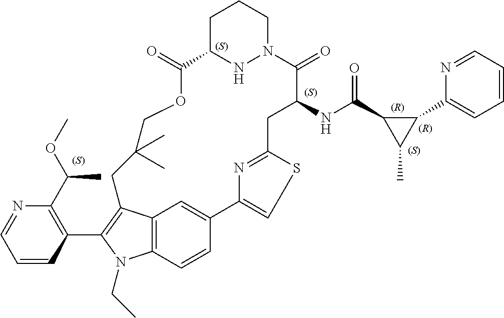 |
| DA 499 | 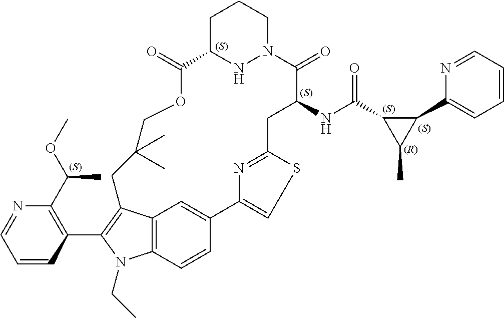 |
| DA 500 | 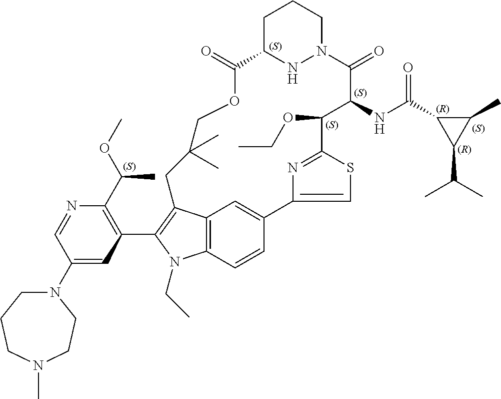 |
| DA 501 | 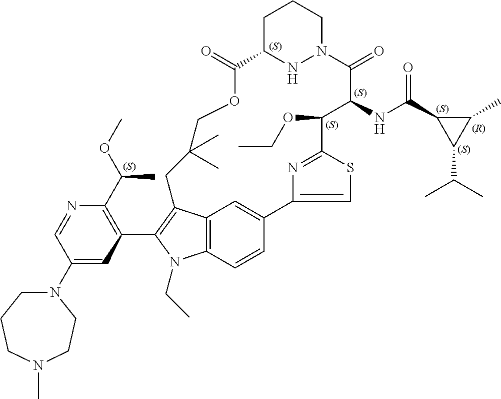 |
| DA 502 | 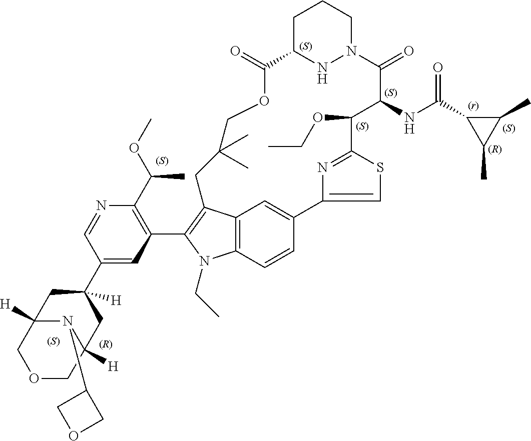 |
| DA 503 | 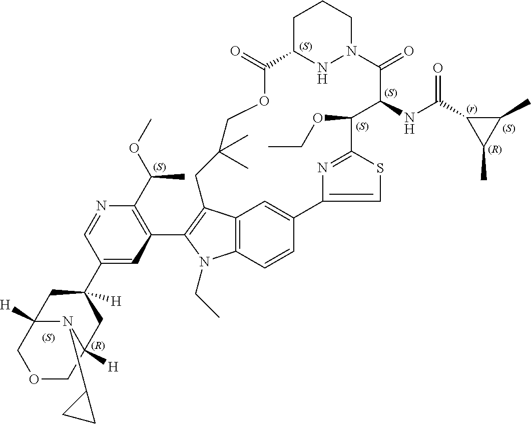 |
| DA 504 | 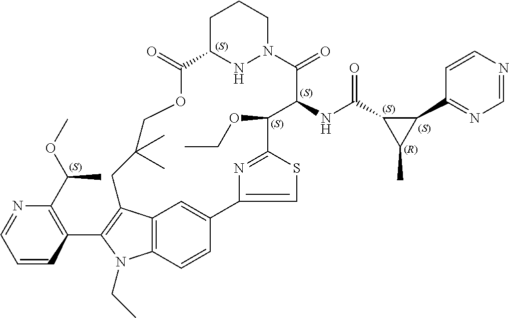 |
| DA 505 | 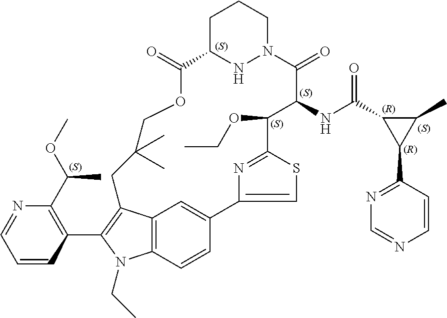 |
| DA 506 | 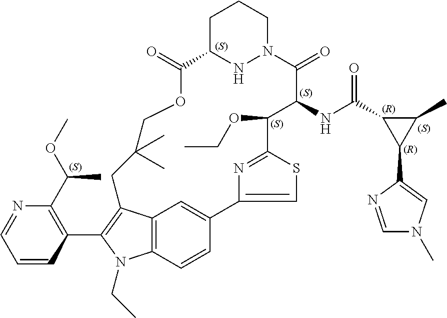 |
| DA 507 | 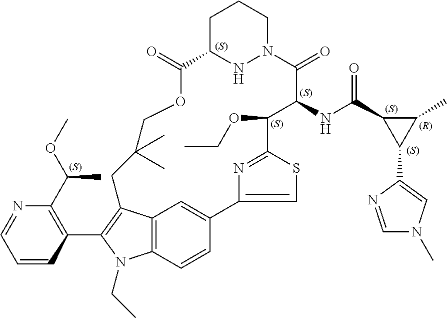 |
| DA 508 | 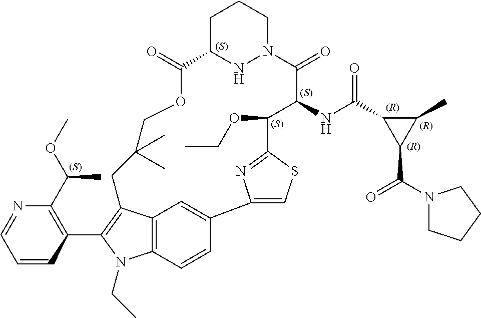 |
| DA 509 | 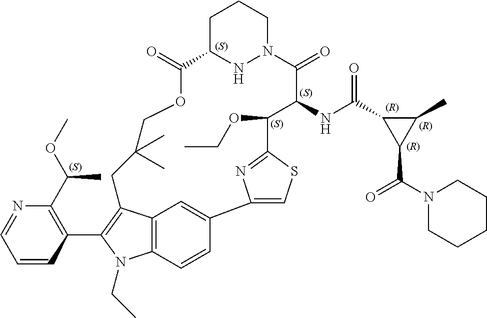 |
| DA 510 | 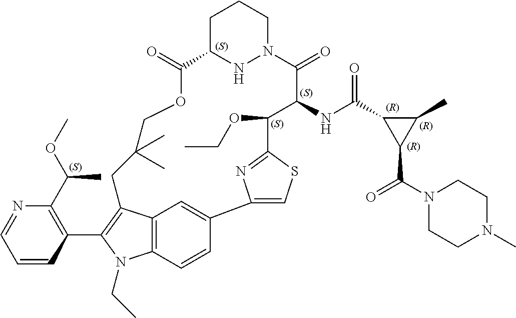 |
| DA 511 | 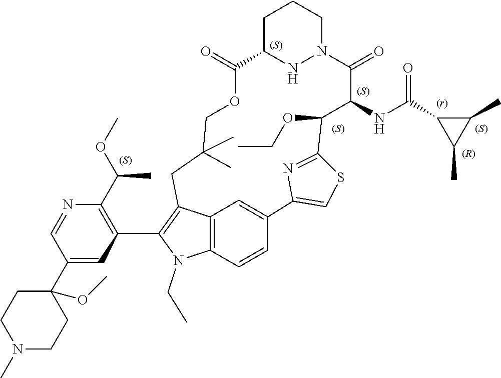 |
| DA 512 | 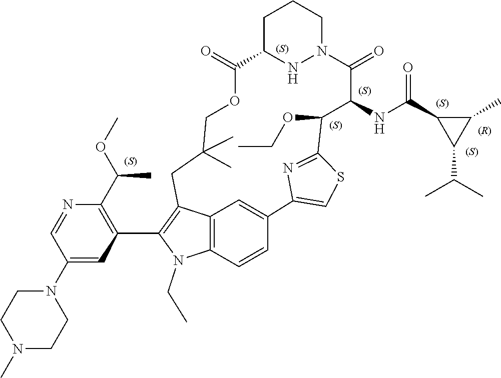 |
| DA 513 | 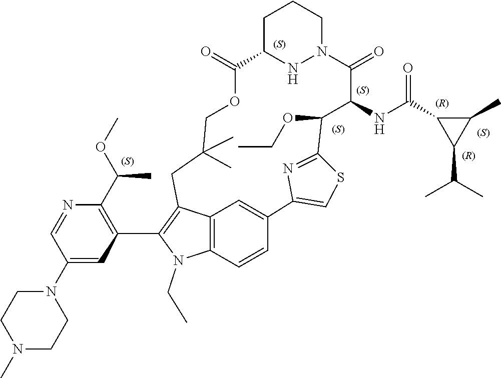 |
| DA 514 | 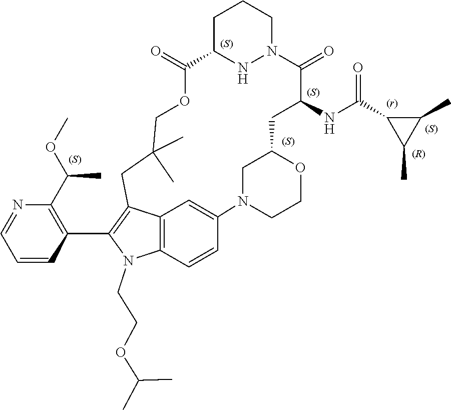 |
| DA 515 | 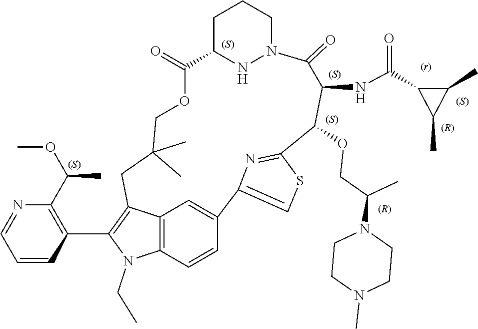 |
| DA 516 | 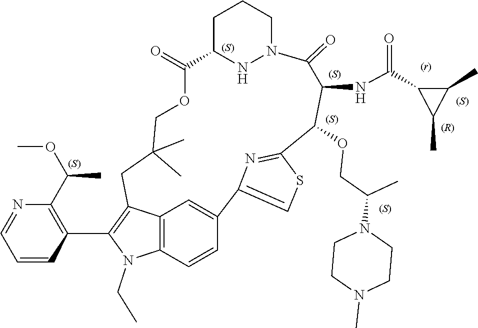 |
| DA 517 | 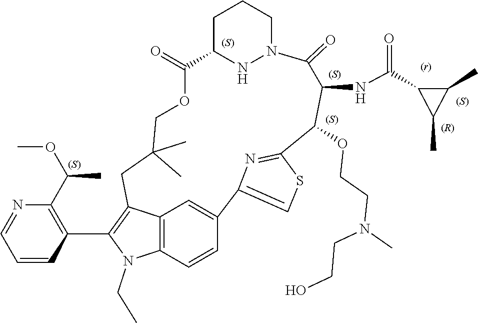 |
| DA 518 | 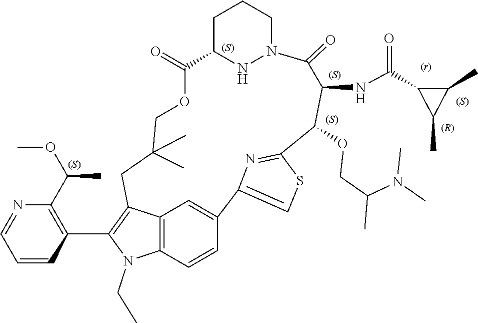 |
| DA 519 | 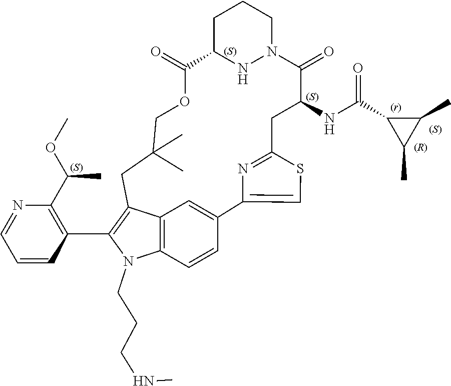 |
| DA 520 | 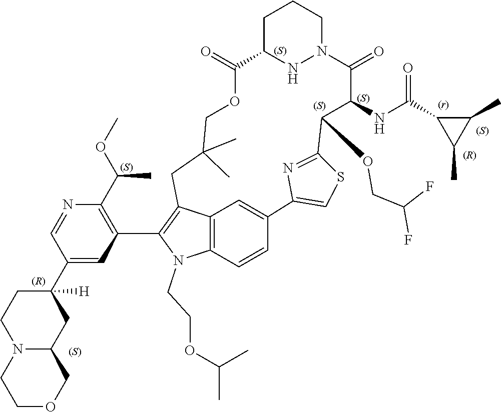 |
| DA 521 | 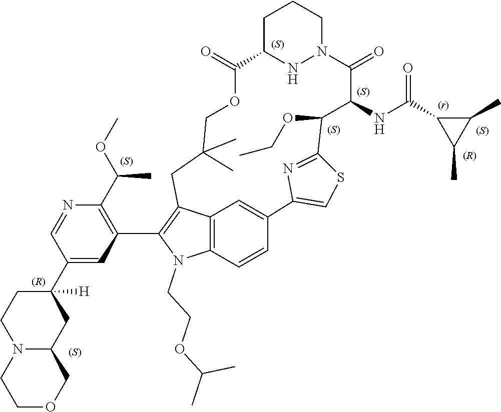 |
| DA 522 | 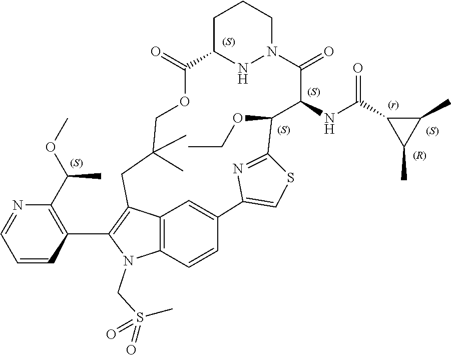 |
| DA 523 | 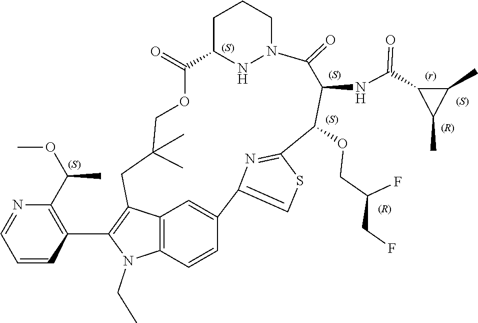 |
| DA 524 | 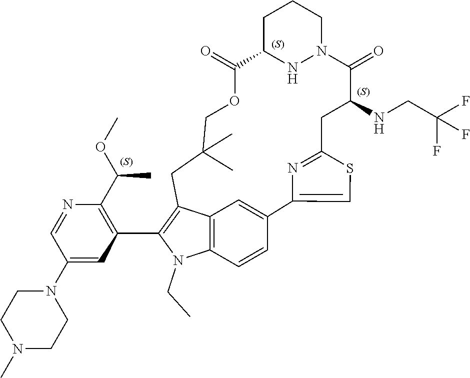 |
| DA 525 | 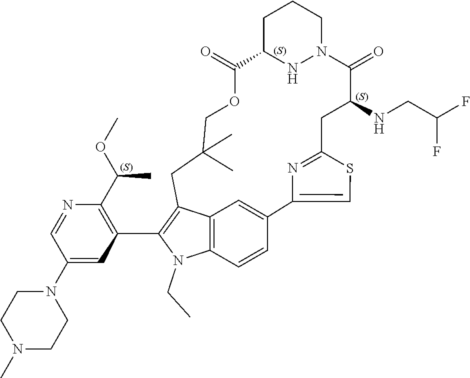 |
| DA 526 | 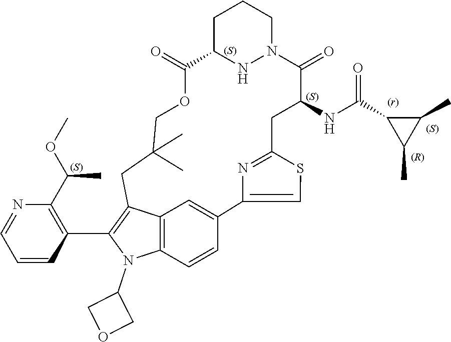 |
| DA 527 | 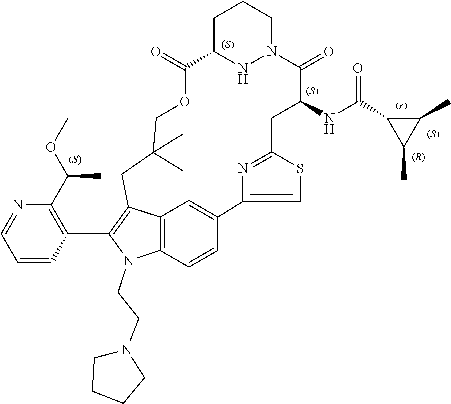 |
| DA 528 | 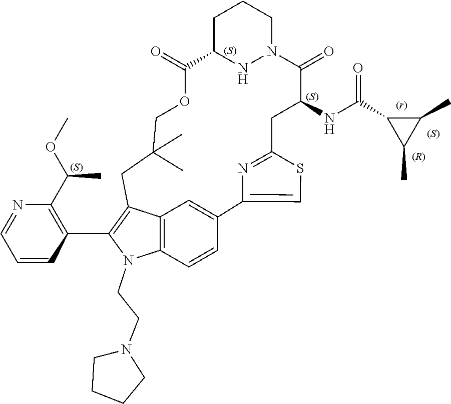 |
| DA 529 | 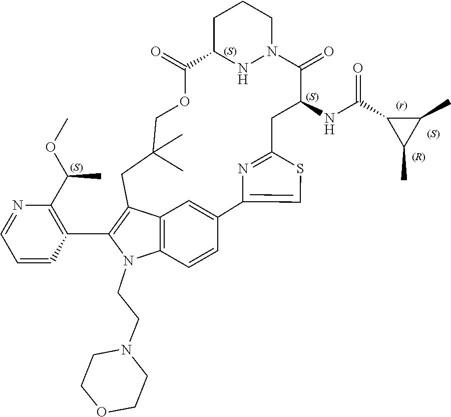 |
| DA 530 | 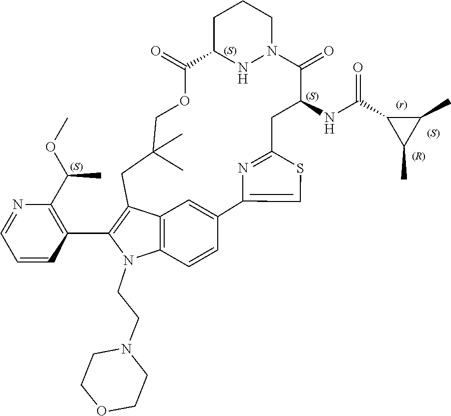 |
| DA 531 | 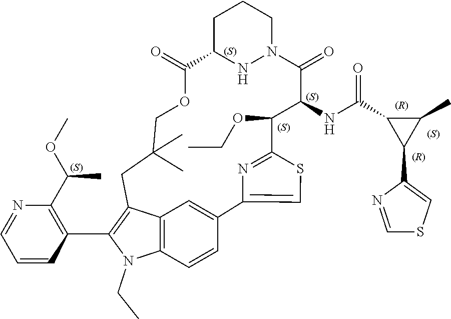 |
| DA 532 | 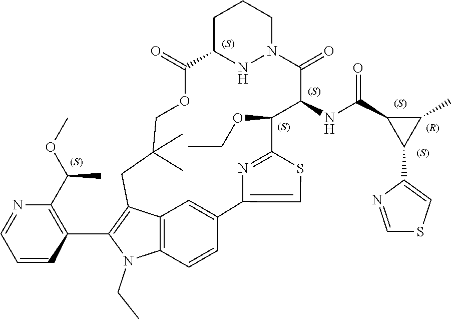 |
| DA 533 | 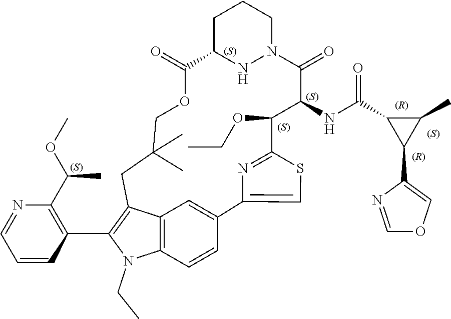 |
| DA 534 | 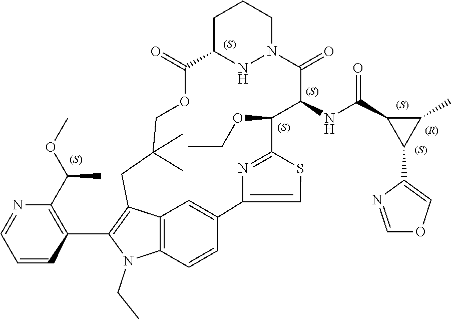 |
| DA 535 | 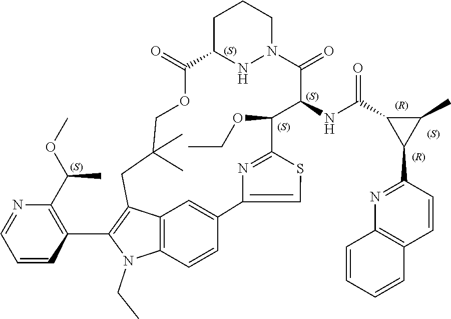 |
| DA 536 | 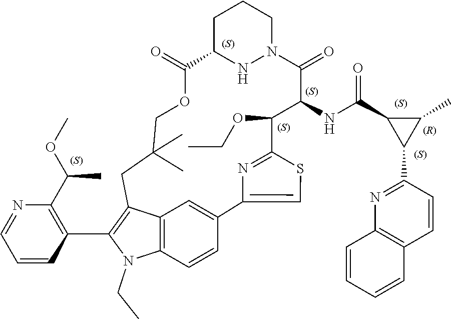 |
| DA 537 | 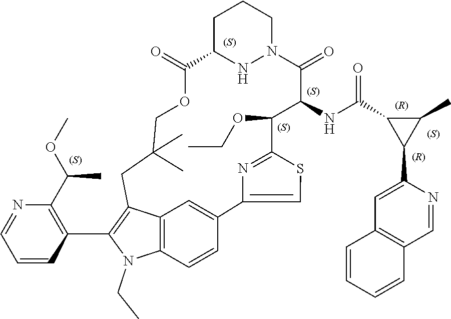 |
| DA 538 | 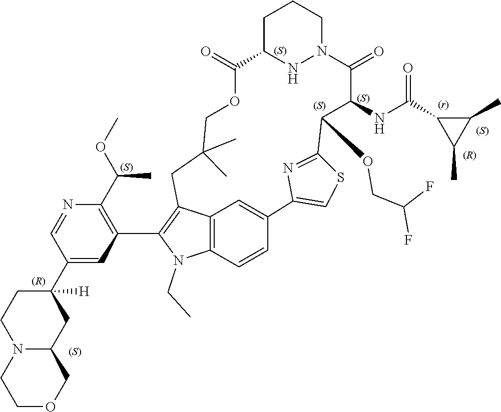 |
| DA 539 | 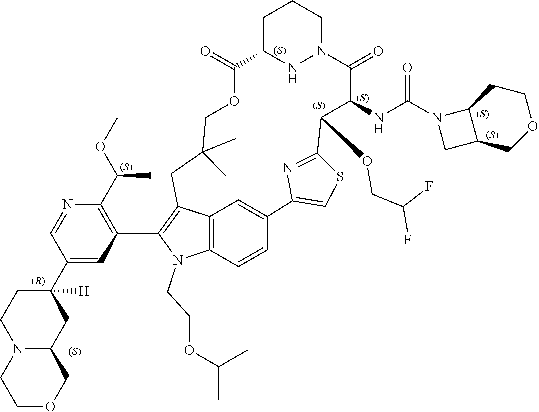 |
| DA 540 | 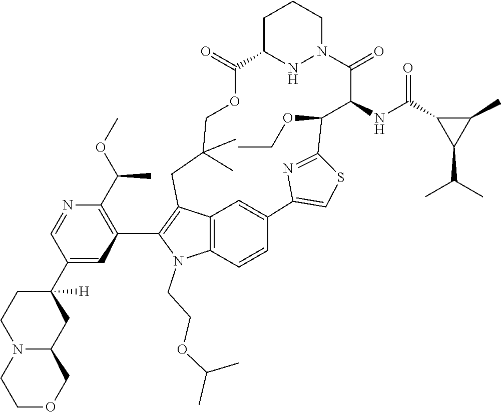 |
| DA 541 | 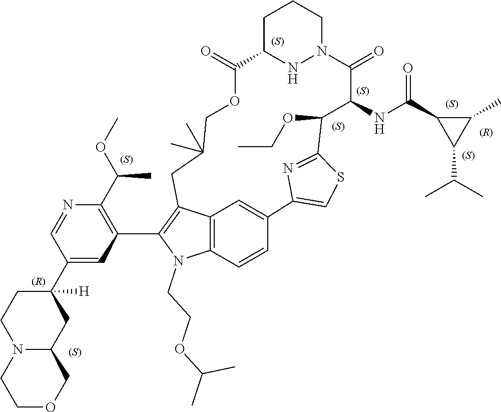 |
| DA 542 | 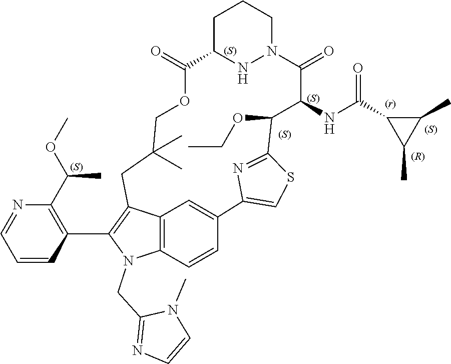 |
| DA 543 | 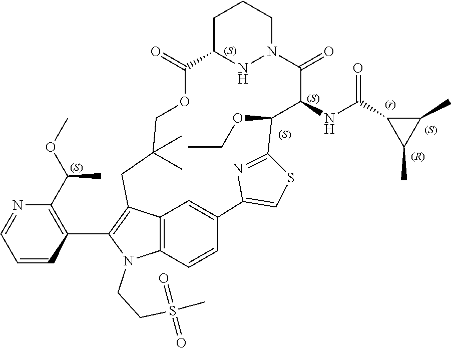 |
| DA 544 | 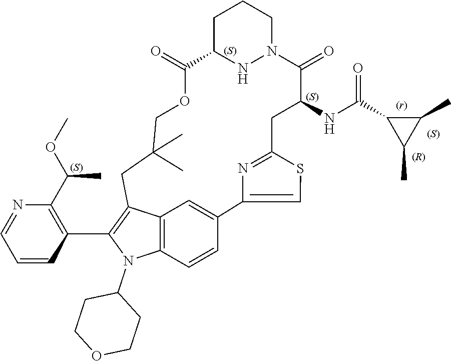 |
| DA 545 | 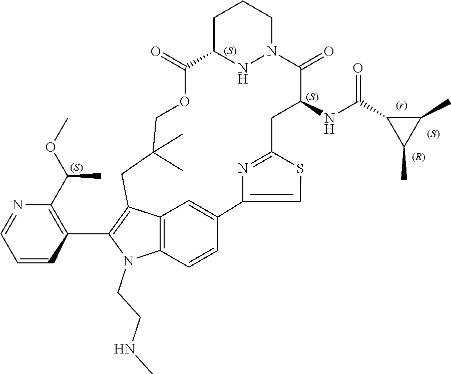 |
| DA 546 | 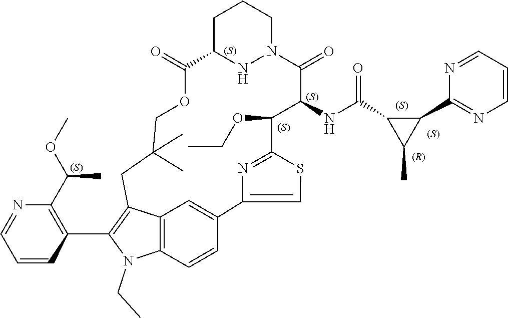 |
| DA 547 | 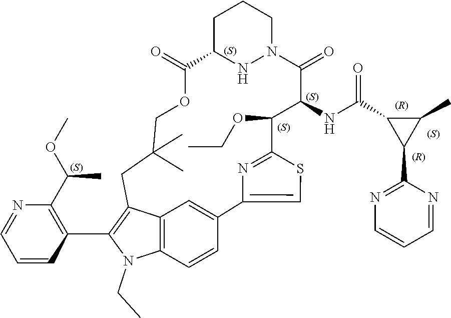 |
| DA 548 | 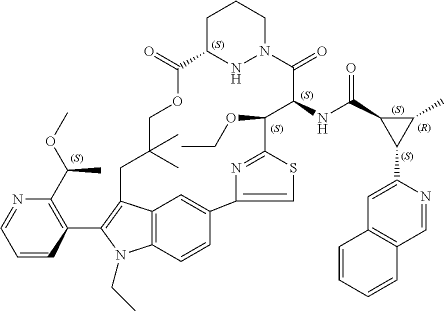 |
| DA 549 | 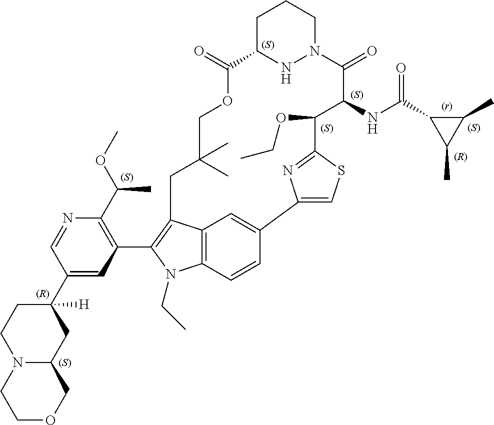 |
| DA 550 | 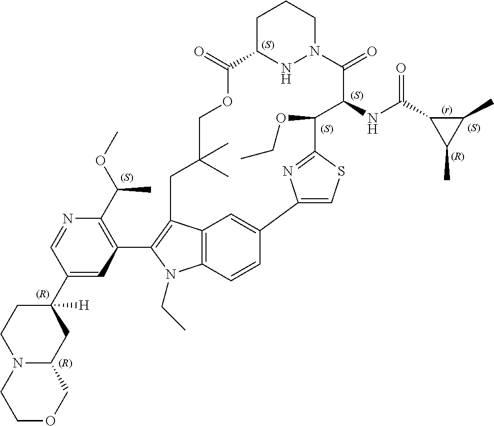 |
| DA 551 | 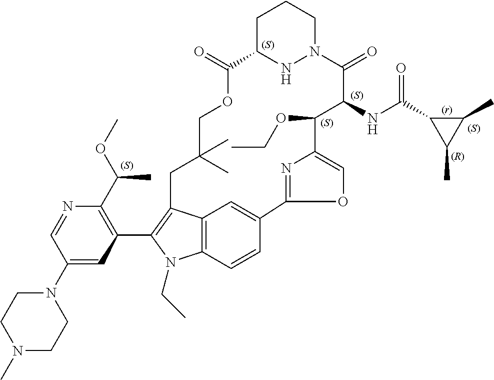 |
| DA 552 | 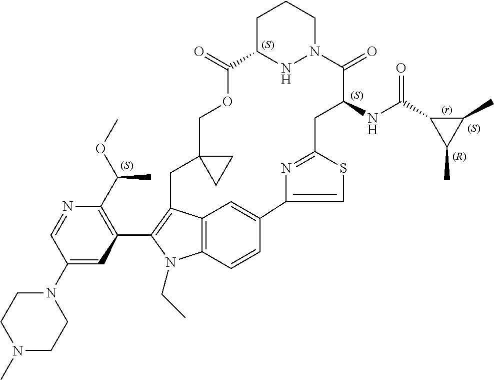 |
| DA 553 | 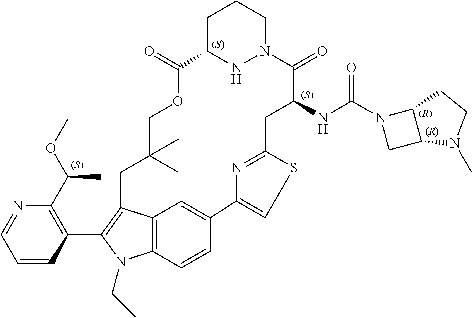 |
| DA 554 | 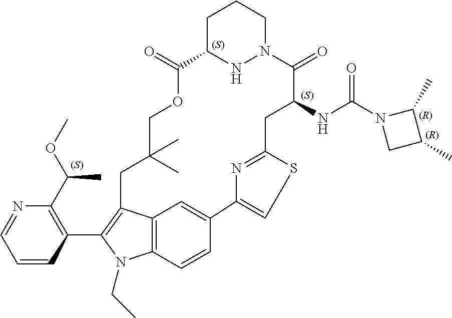 |
| DA 555 | 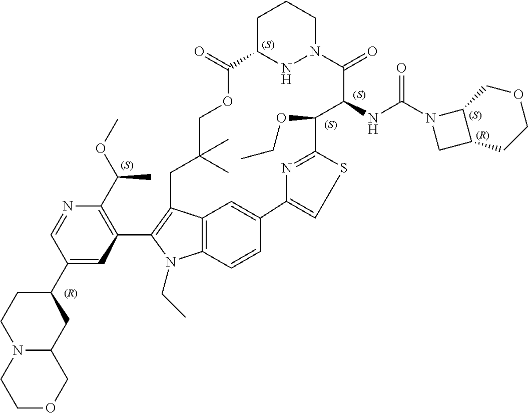 |
| DA 556 | 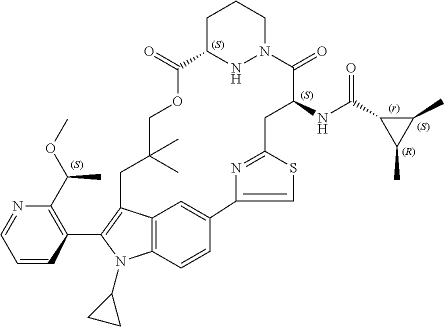 |
| DA 557 | 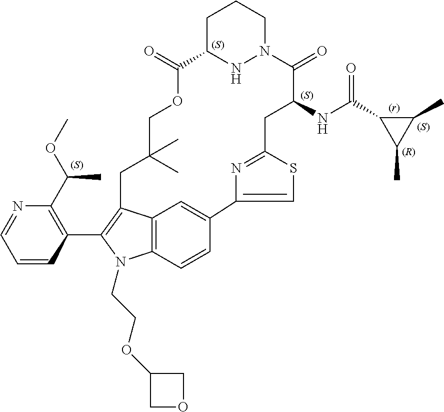 |
| DA 558 | 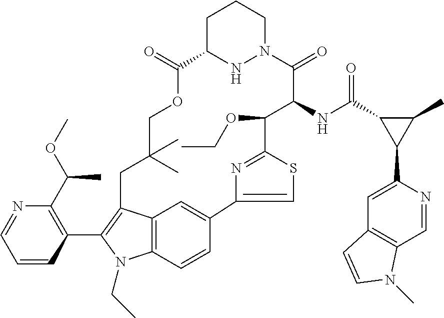 |
| DA 559 | 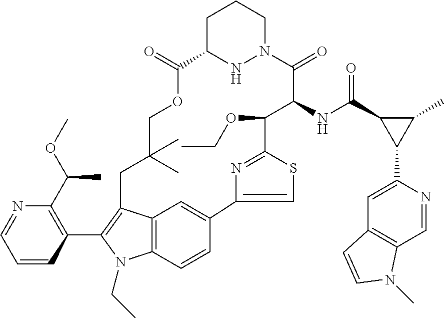 |
| DA 560 | 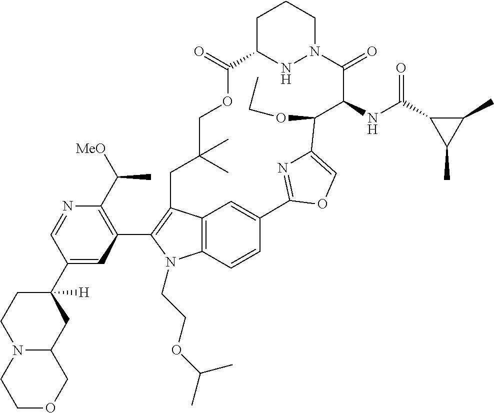 |
| DA 561 | 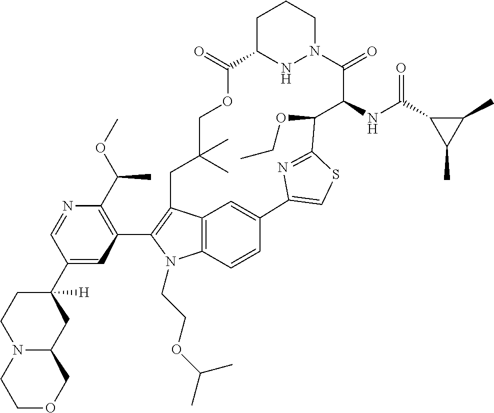 |
| DA 562 | 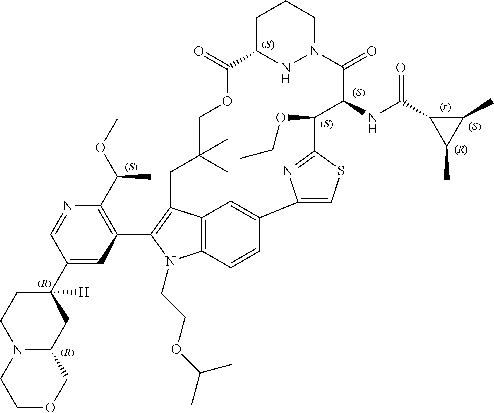 |
| DA 563 | 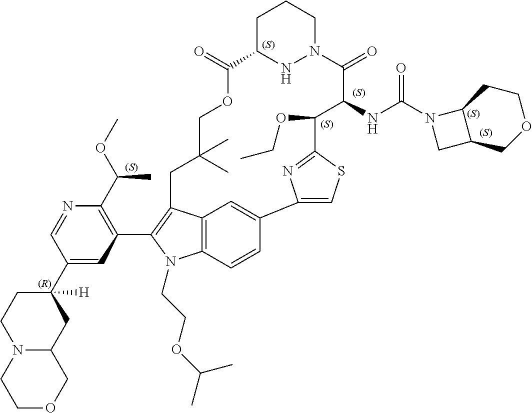 |
| DA 564 | 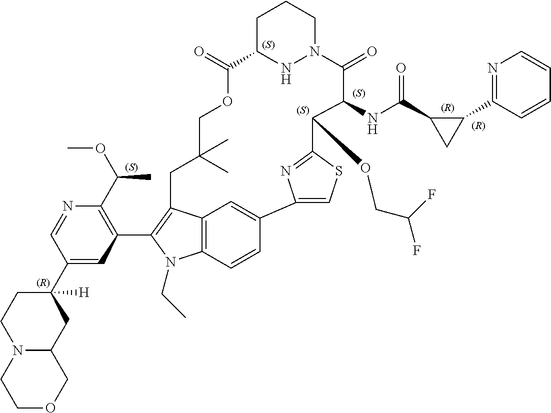 |
| DA 565 | 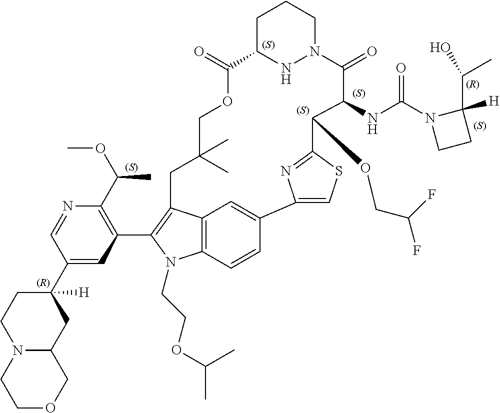 |
| DA 566 | 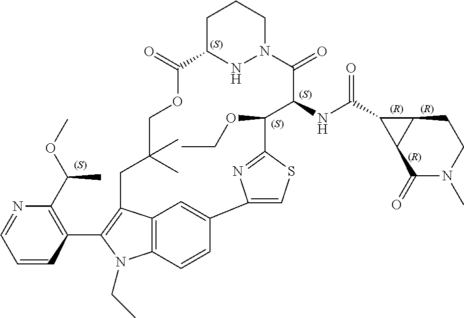 |
| DA 567 | 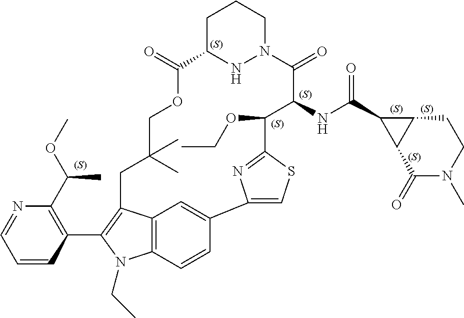 |
| DA 568 | 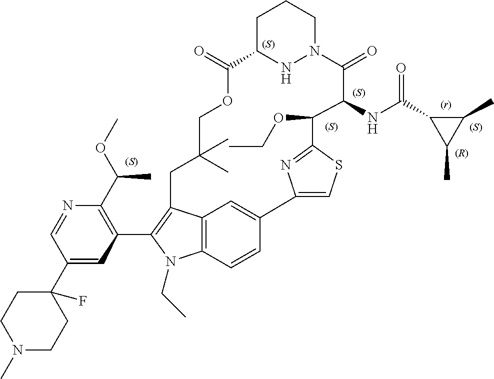 |
| DA 569 | 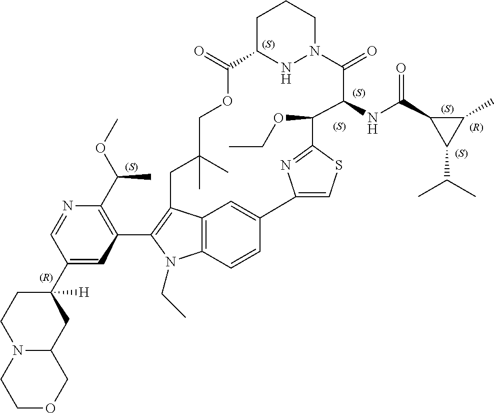 |
| DA 570 | 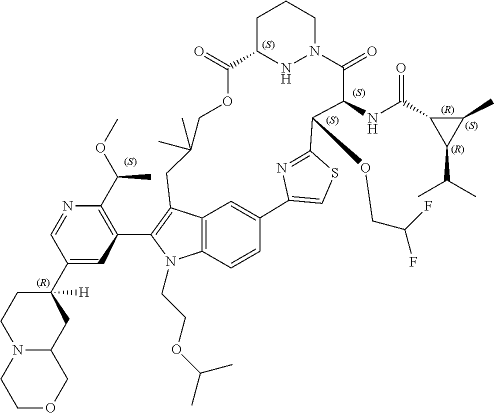 |
| DA 571 | 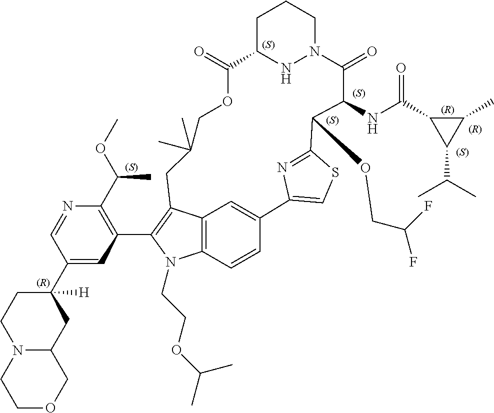 |
| DA 572 | 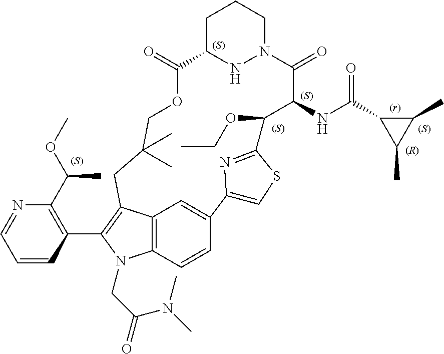 |
| DA 573 | 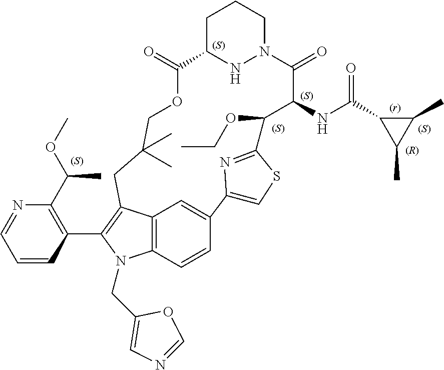 |
| DA 574 | 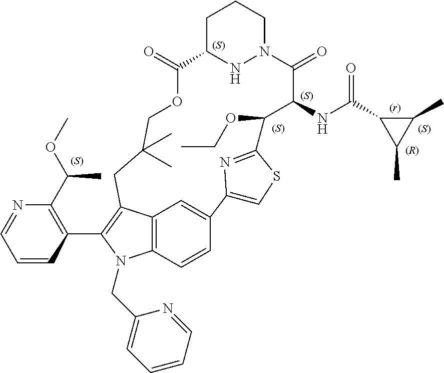 |
| DA 575 | 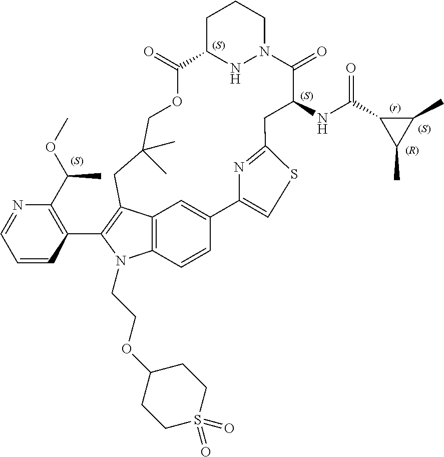 |
| DA 576 | 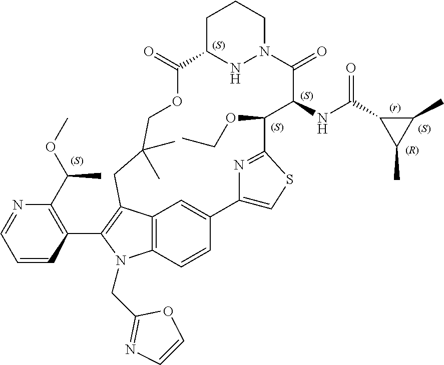 |
| DA 577 | 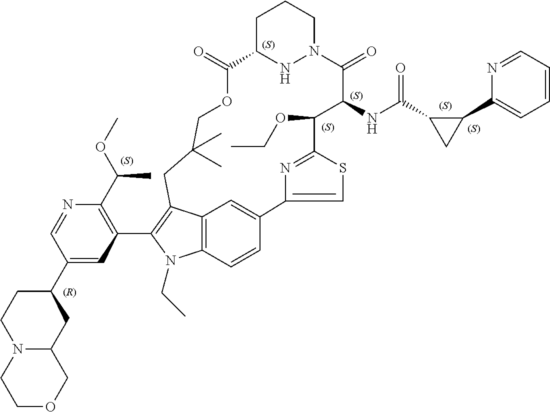 |
| DA 578 | 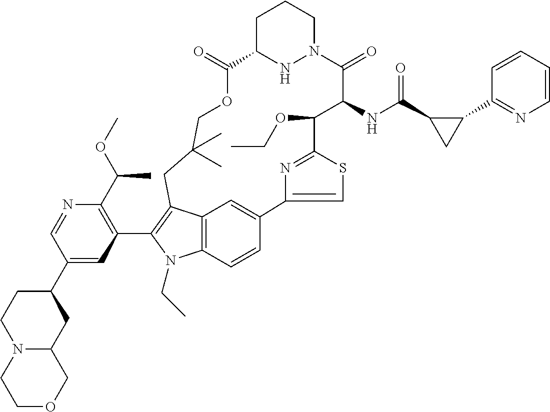 |
| DA 579 | 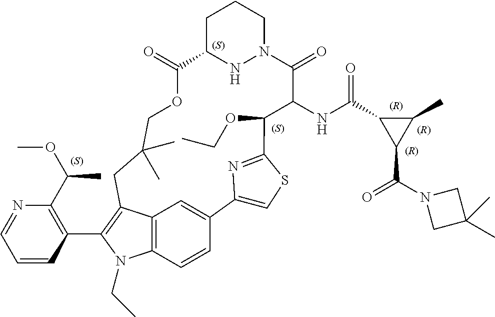 |
| DA 580 | 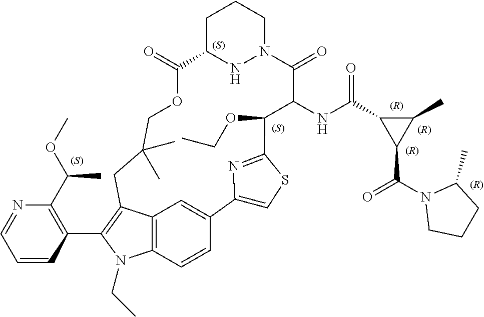 |
| DA 581 | 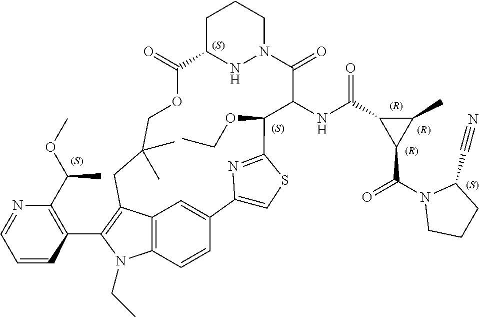 |
| DA 582 | 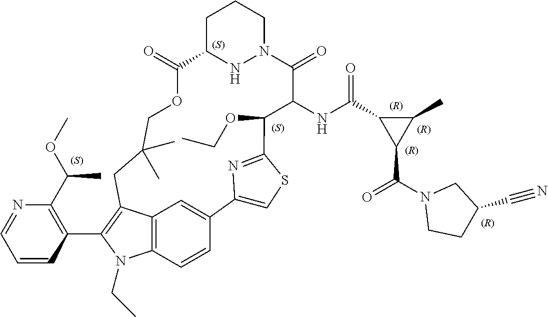 |
| DA 583 | 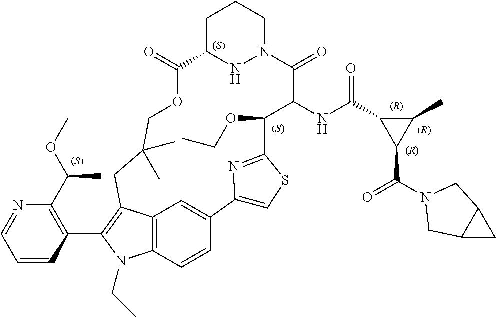 |
| DA 584 | 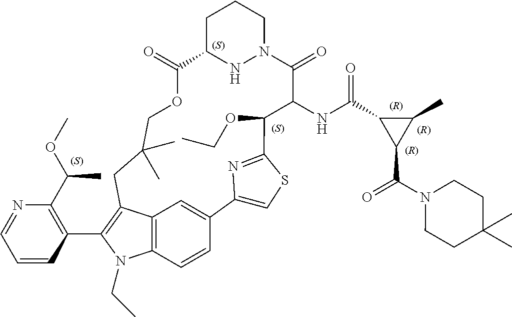 |
| DA 585 | 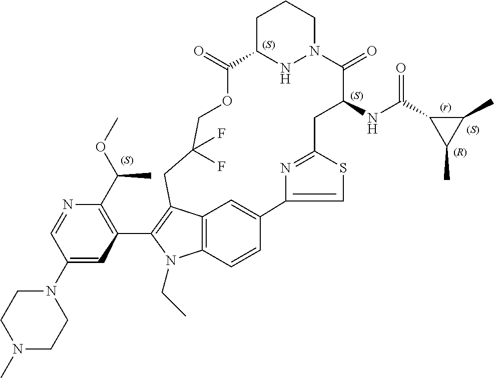 |
| DA 586 | 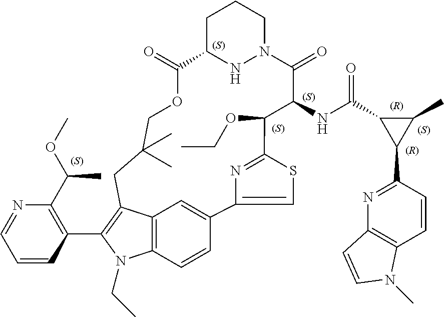 |
| DA 587 | 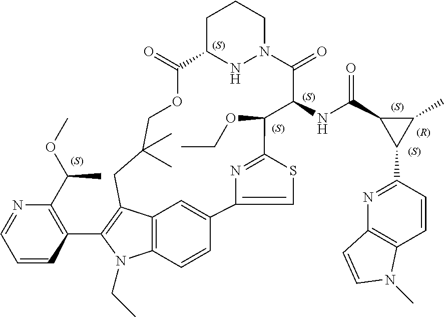 |
| DA 588 | 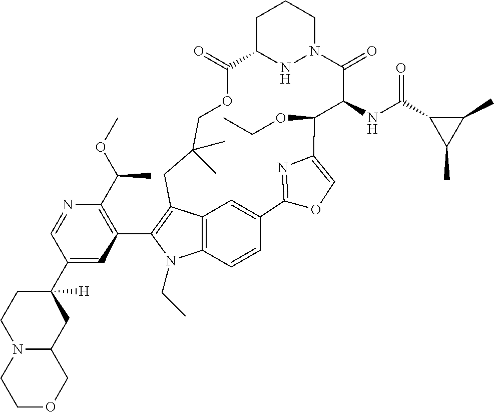 |
| DA 589 | 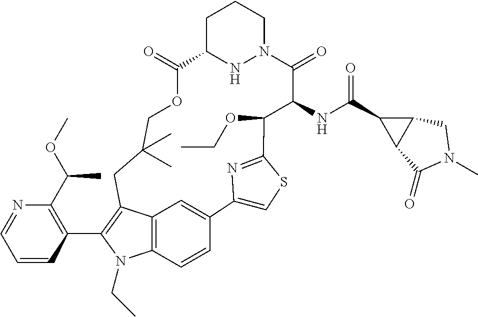 |
| DA 590 | 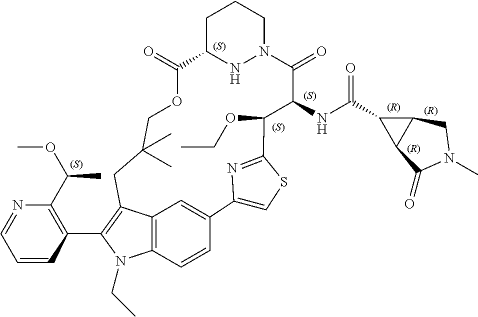 |
| DA 591 | 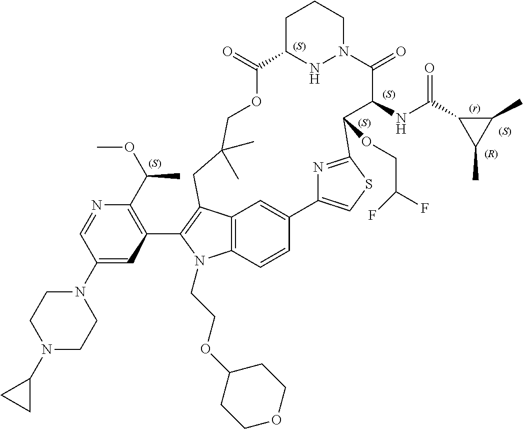 |
| DA 592 | 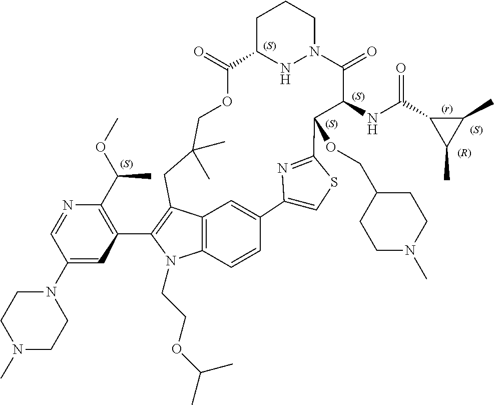 |
| DA 593 | 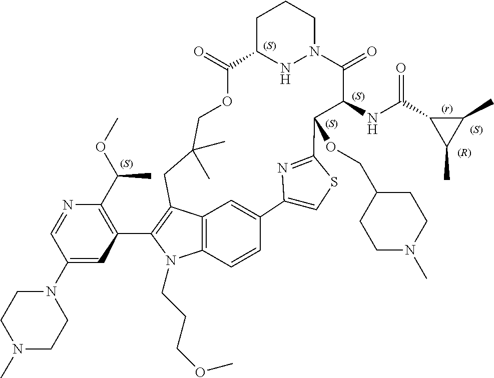 |
| DA 594 | 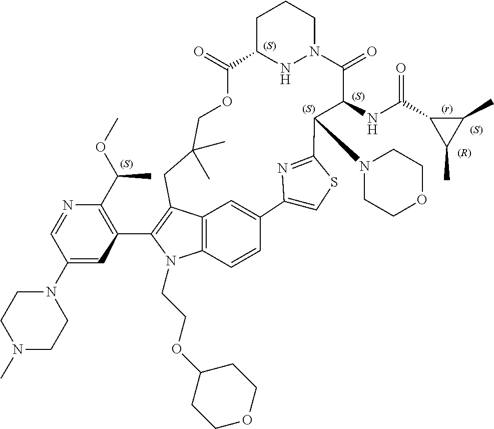 |
| DA 595 | 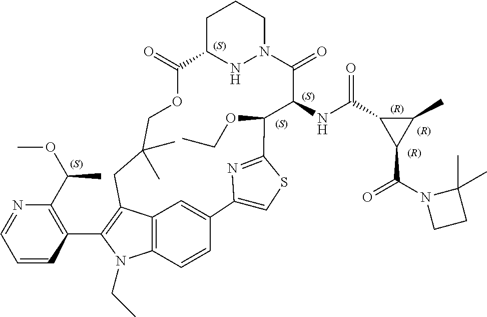 |
| DA 596 | 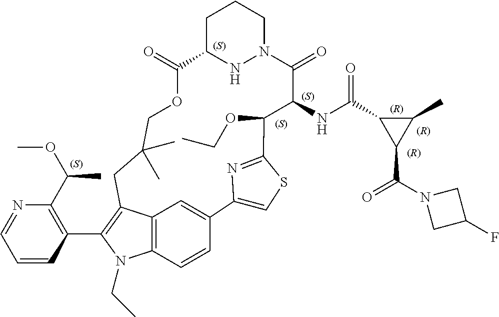 |
| DA 597 | 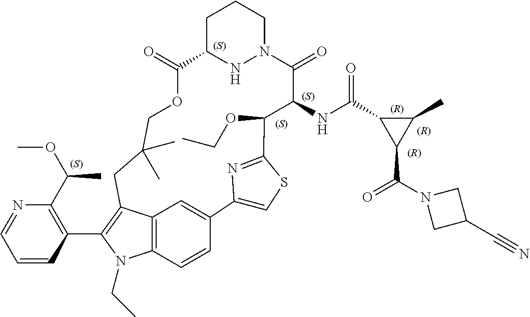 |
| DA 598 | 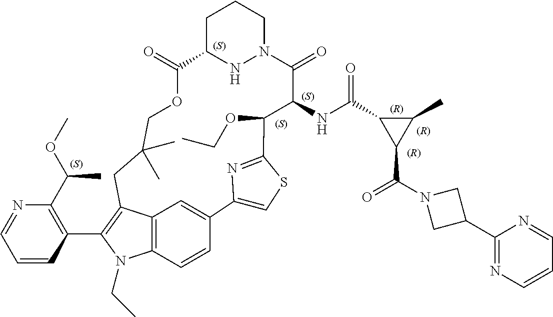 |
| DA 599 | 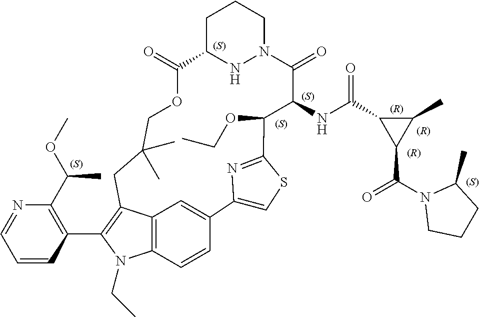 |
| DA 600 | 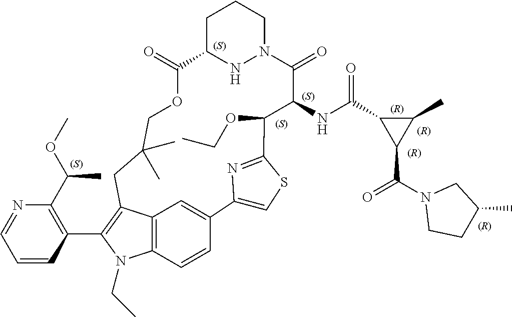 |
| DA 601 | 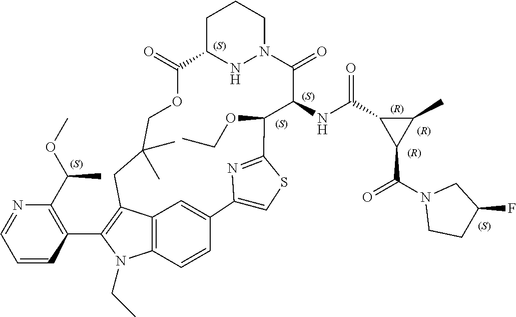 |
| DA 602 | 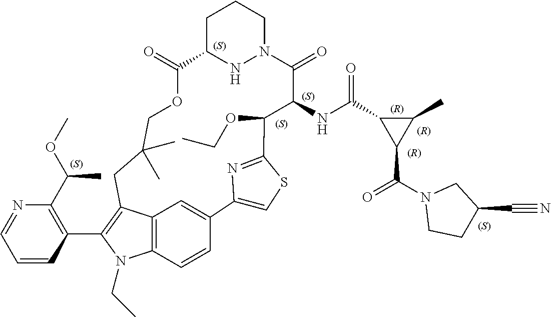 |
| DA 603 | 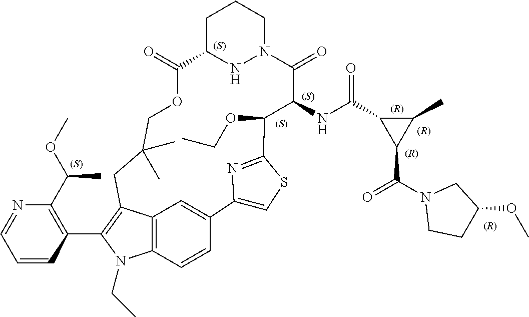 |
| DA 604 | 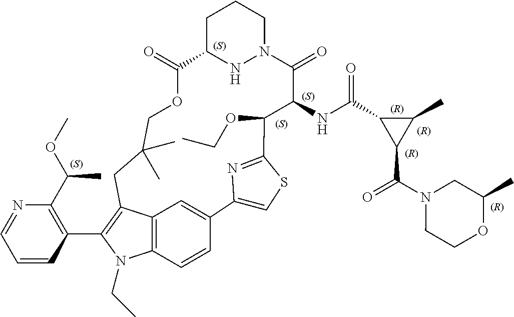 |
| DA 605 | 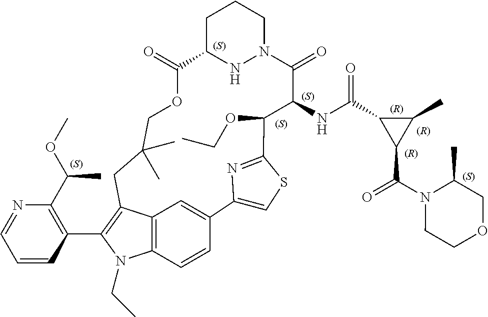 |
| DA 606 | 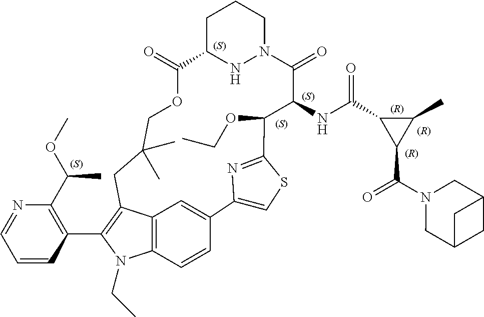 |
| DA 607 | 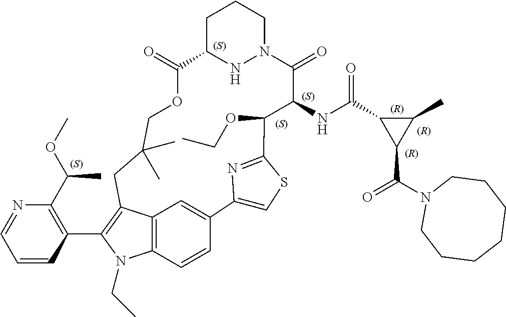 |
| DA 608 | 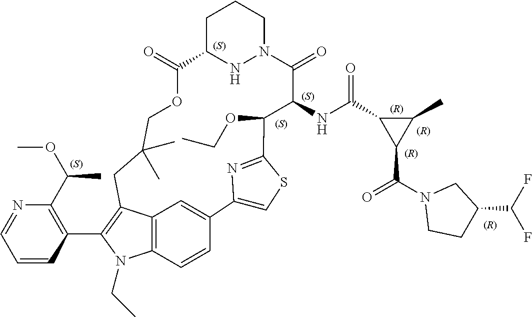 |

Note
that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereochemistry of such diastereomer may not be known.

In some embodiments, the RAS(ON) inhibitor is a compound selected from Table D2, or a pharmaceutically acceptable salt or stereoisomer thereof. In some embodiments, the RAS(ON) inhibitor is a compound selected from Table D2, or a pharmaceutically acceptable salt or atropisomer thereof.

In some embodiments, the RAS(ON) inhibitor is not a compound selected from Table D2. In some embodiments, the RAS(ON) inhibitor is not a compound selected from Table D2, or a pharmaceutically acceptable salt or stereoisomer thereof. In some embodiments, the RAS(ON) inhibitor is not a compound selected from Table D2, or a pharmaceutically acceptable salt or atropisomer thereof.

TABLE D2
Certain Compounds

| Ex. # | Structure |
|---|---|
| DB1 | 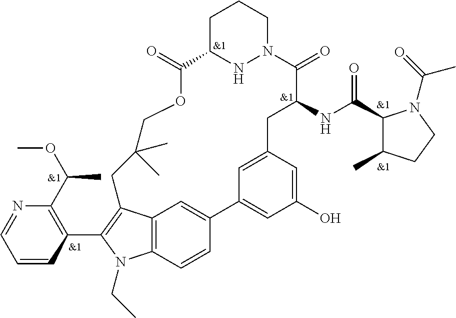 |
| DB2 | 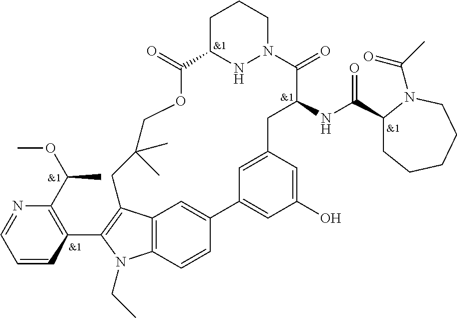 |
| DB3 | 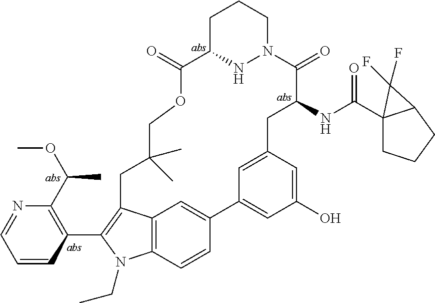 |
| DB4 | 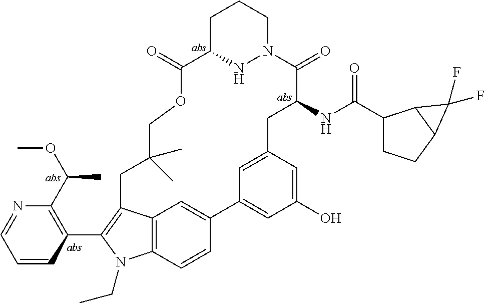 |
| DB5 | 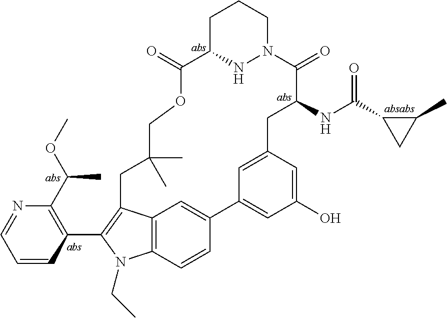 |
| DB6 | 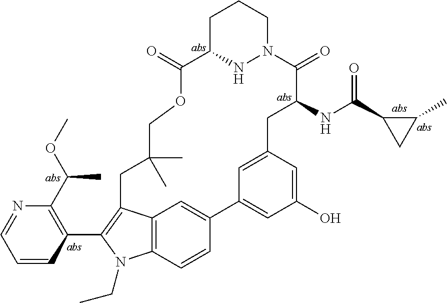 |
| DB7 | 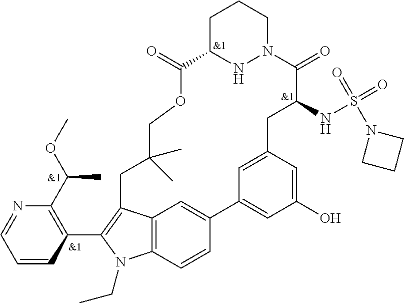 |
| DB8 | 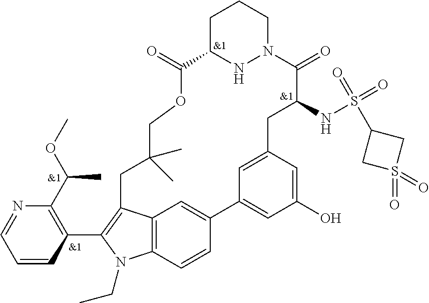 |
| DB9 | 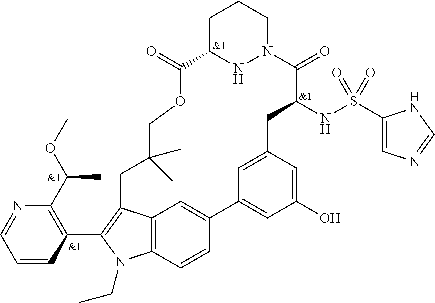 |
| DB10 | 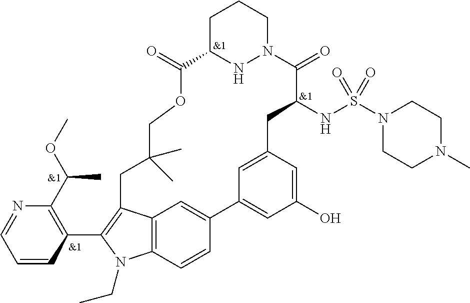 |
| DB11 | 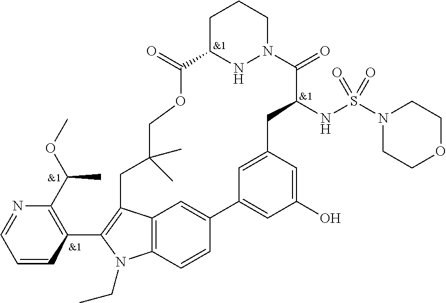 |
| DB12 | 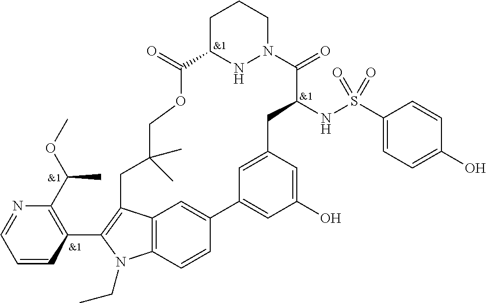 |
| DB13 | 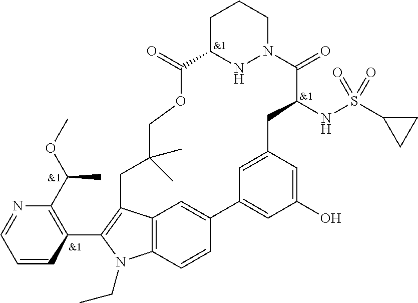 |
| DB14 | 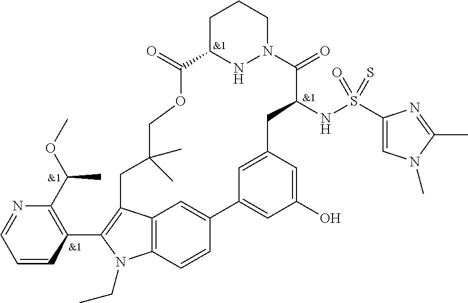 |
| DB15 | 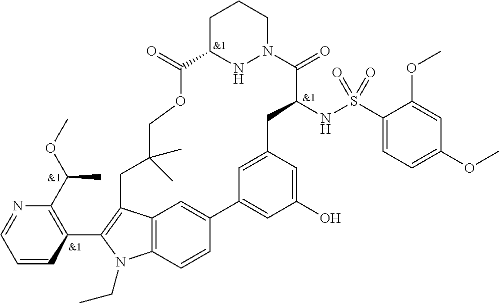 |
| DB16 | 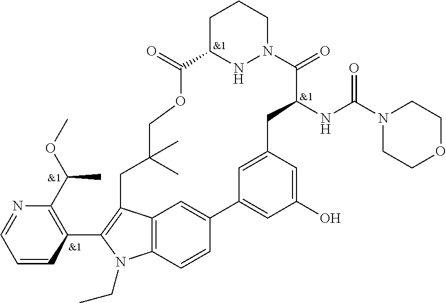 |
| DB17 | 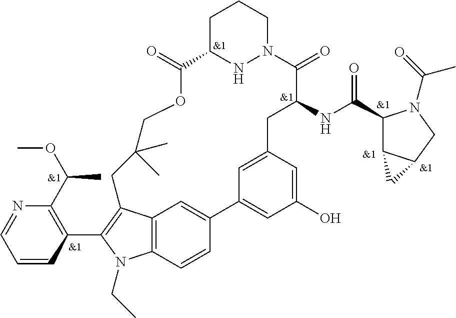 |
| DB18 | 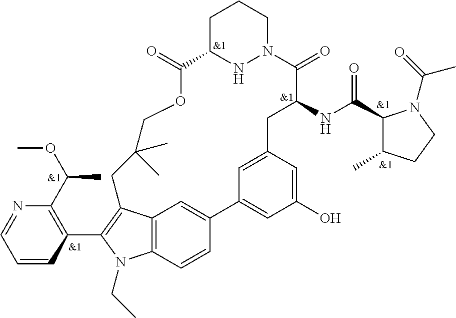 |
| DB19 | 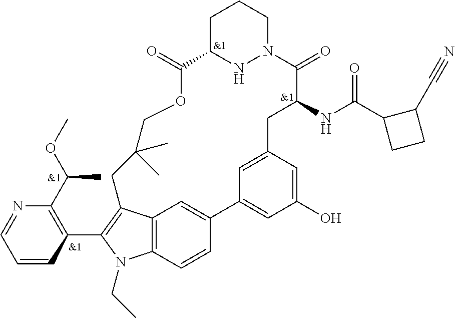 |
| DB20 | 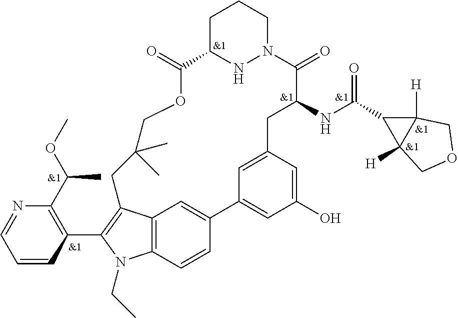 |
| DB21 | 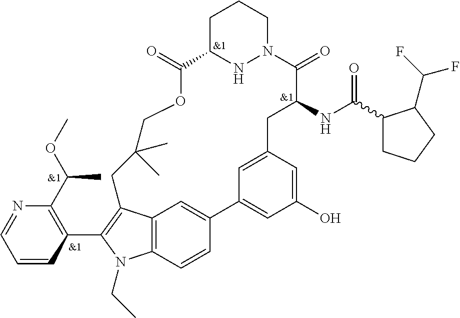 |
| DB22 | 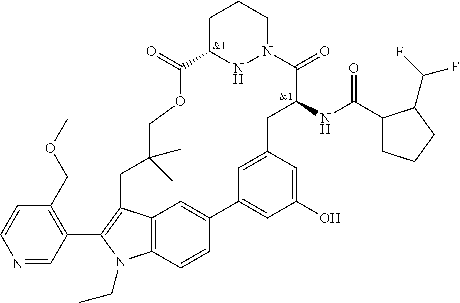 [SMILES: CCn1c(-c2cnccc2COC)c2c3cc(ccc31)-c1cc(O)cc(c1)C[C@H](NC(=O)C1CCCC1C(F)F)C(=O)N1CCC[C@H](N1)C(=O)OCC(C)(C)C2] |
| DB23 | 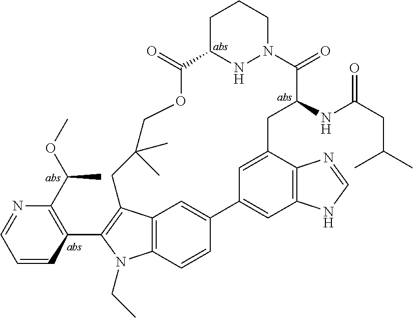 |
| DB24 | 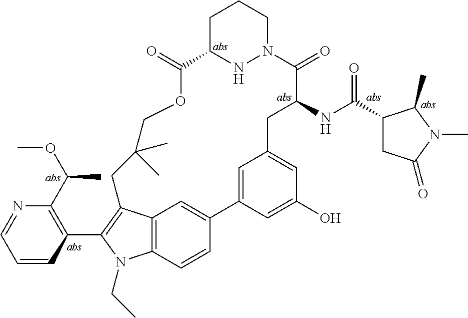 |
| DB25 | 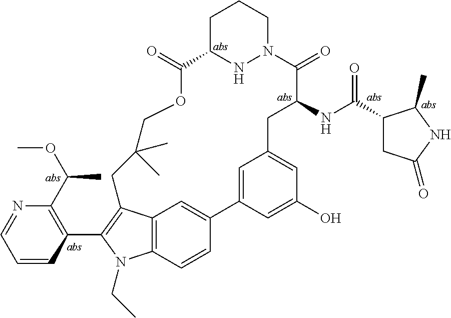 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereochemistry of such diastereomer may not be known.

In some embodiments, a compound of the present invention is a compound selected from Table D3 (e.g., DC1-DC20 or DC1-DC21), or a pharmaceutically acceptable salt or stereoisomer thereof. In some embodiments, a compound of the present invention is a compound selected from Table D3 (e.g., DC1-DC20 or DC1-DC21), or a pharmaceutically acceptable salt or atropisomer thereof.

In some embodiments, a compound of the present invention is not a compound selected from Table D3 (e.g., DC1-DC20 or DC1-DC21). In some embodiments, a compound of the present invention is not a compound selected from Table D3 (e.g., DC1-DC20 or DC1-DC21), or a pharmaceutically acceptable salt or stereoisomer thereof. In some embodiments, a compound of the present invention is not a compound selected from Table D3 (e.g., DC1-DC20 or DC1-DC21), or a pharmaceutically acceptable salt or atropisomer thereof.

TABLE D3
Certain Compounds

| Ex. | |
|---|---|
| # | Structure |
| DC1 | 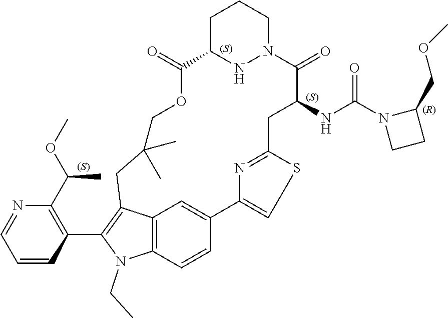 |
| DC2 | 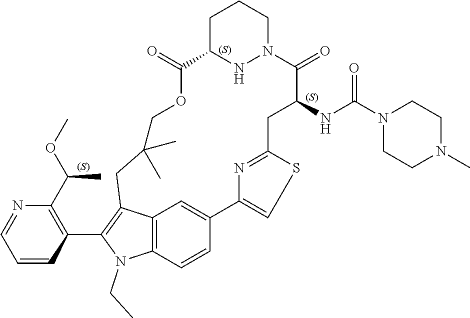 |
| DC3 | 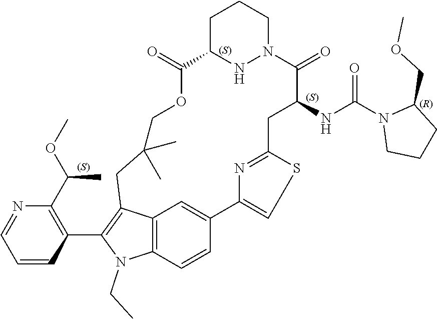 |
| DC4 | 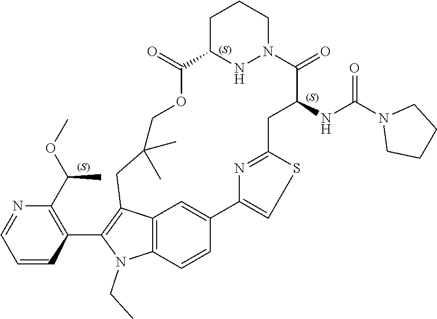 |
| DC5 | 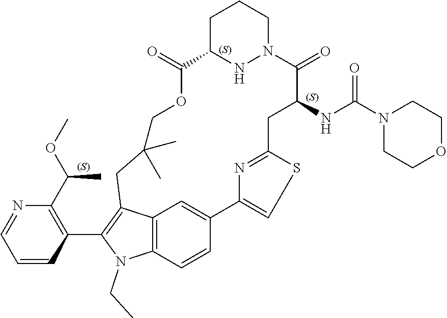 |
| DC6 | 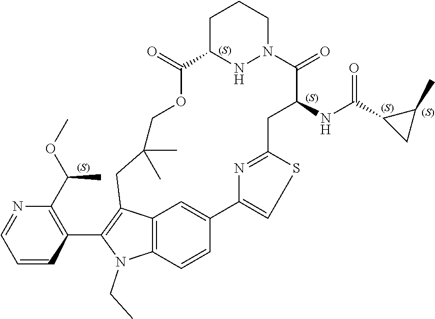 |
| DC7 | 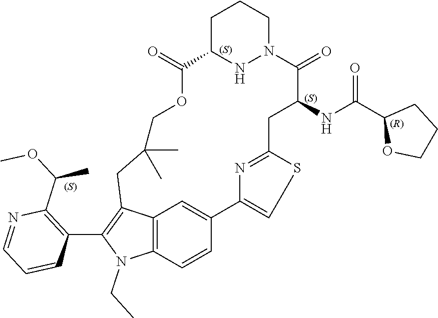 |
| DC8 | 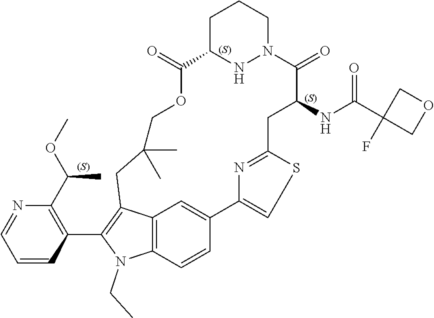 |
| DC9 | 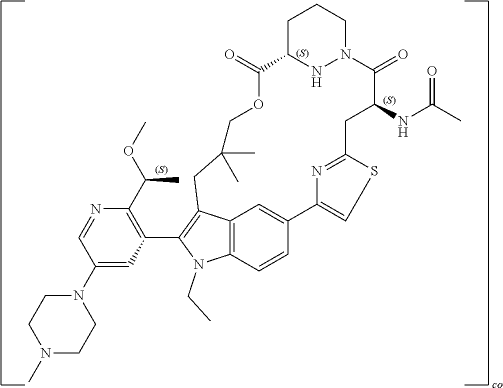 |
| DC10 | 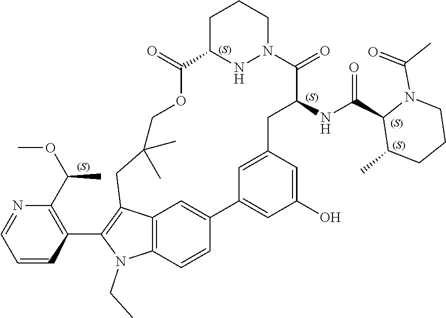 |
| DC11 | 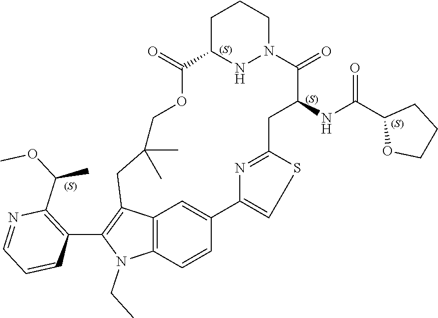 |
| DC12 | 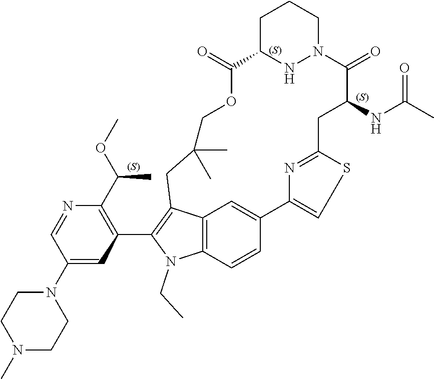 |
| DC13 | 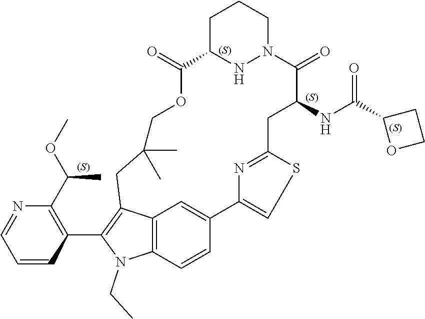 |
| DC14 | 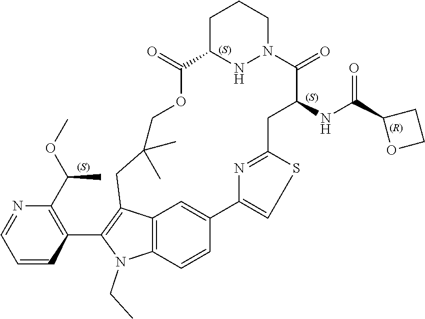 |
| DC15 | 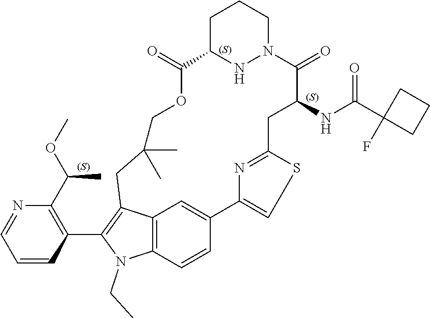 |
| DC16 | 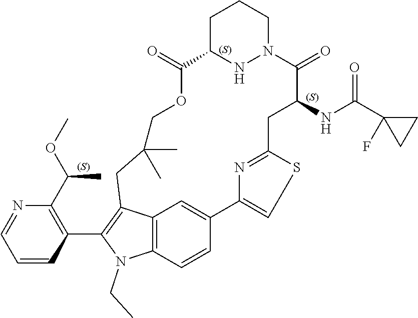 |
| DC17 | 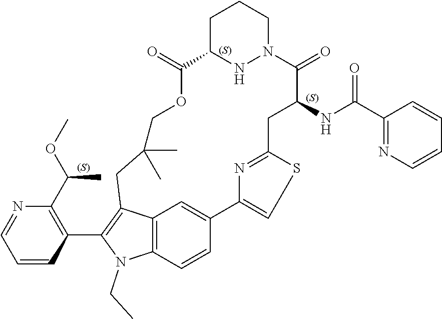 |
| DC18 | 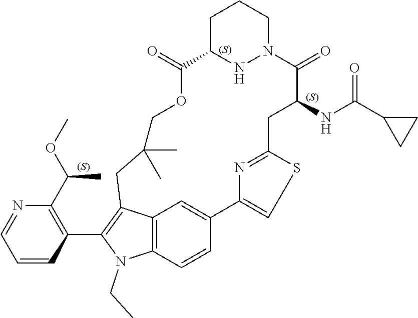 |
| DC19 | 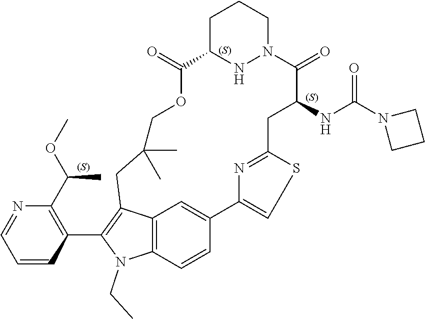 |
| DC20 | 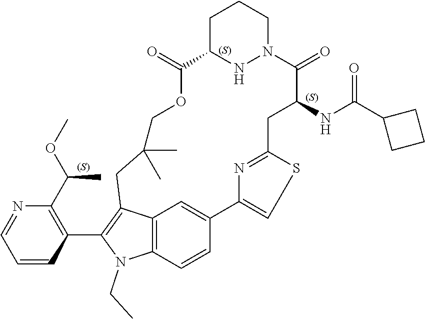 |
| DC21 | 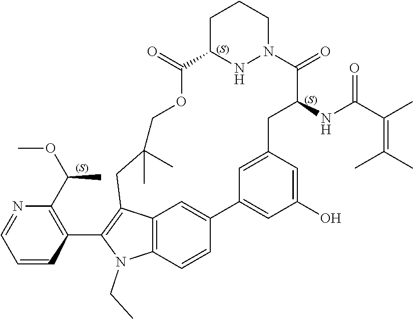 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated. Any compound shown in brackets indicates that the compound is a diastereomer, and the absolute stereo-
chemistry of such diastereomer may not be known.

The compounds described herein in Tables D1a, D1b, D2, and D3 may be made from commercially available starting materials or synthesized using known organic, inorganic, or enzymatic processes.

The compounds of the present invention in Tables D1a, D1b, D2, and D3 can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below and in WO 2022/060836.

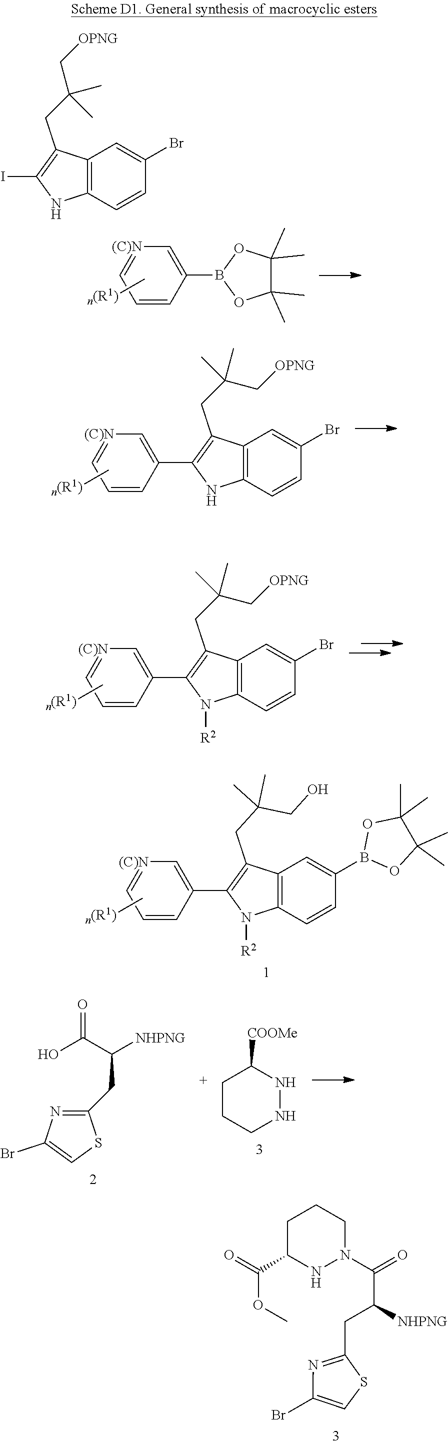

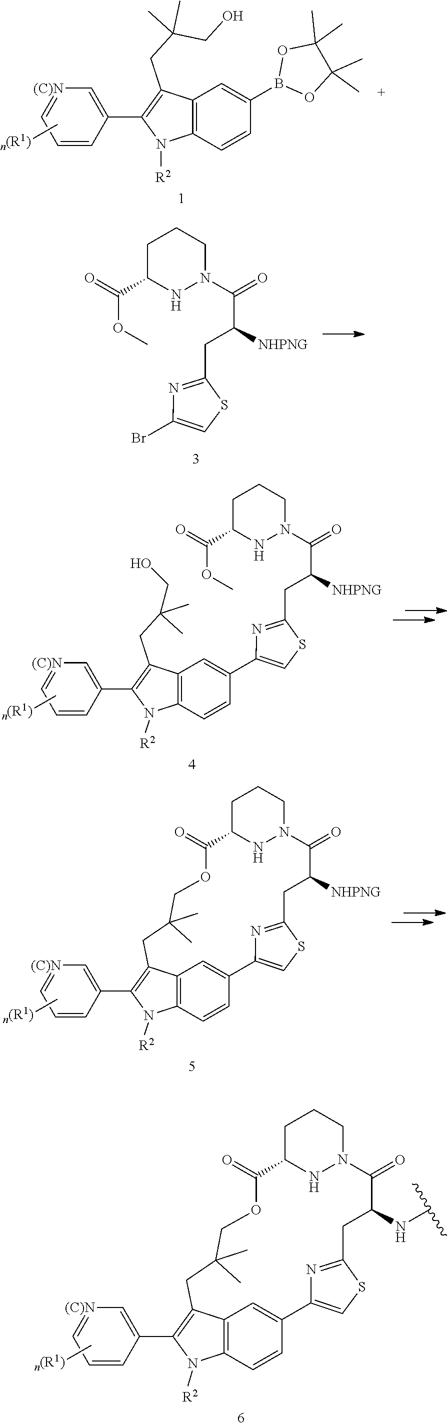

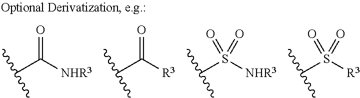

A general synthesis of macrocyclic esters is outlined in Scheme D1. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl-amino-3-(4-bromothiazol-2-yl)propanoyl)hexahydropyridazine-3-carboxylate (3) can be prepared via coupling of (S)-2-amino-3-(4-bromothiazol-2-yl)propanoic acid (2) with methyl (S)-hexahydropyridazine-3-carboxylate.

The final macrocyclic esters can be made by coupling of methyl-amino-3-(4-bromothiazol-2-yl)propanoyl)hexahydropyridazine-3-carboxylate (3) and an appropriately substituted indolyl boronic ester (1) in the presence of Pd catalyst followed by hydrolysis and macrolactonization steps to result in an appropriately protected macrocyclic intermediate (5). Deprotection and coupling with an appropriately substituted carboxylic acid (or other coupling partner) can result in a macrocyclic product. Additional deprotection or functionalization steps could be required to produce a final compound 6.

Further, with respect to Scheme D1, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

### Scheme D2. Alternative General Synthesis of Macrocyclic Esters

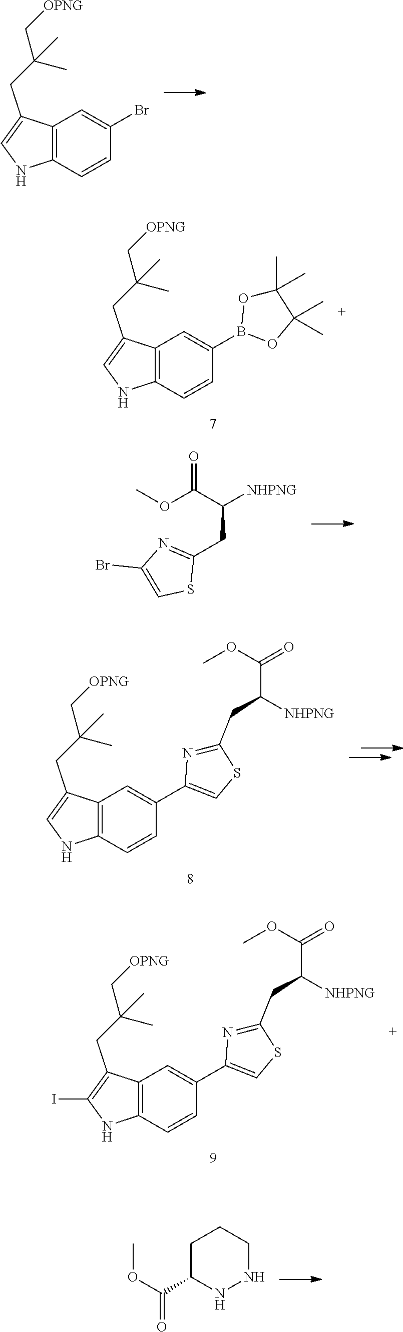

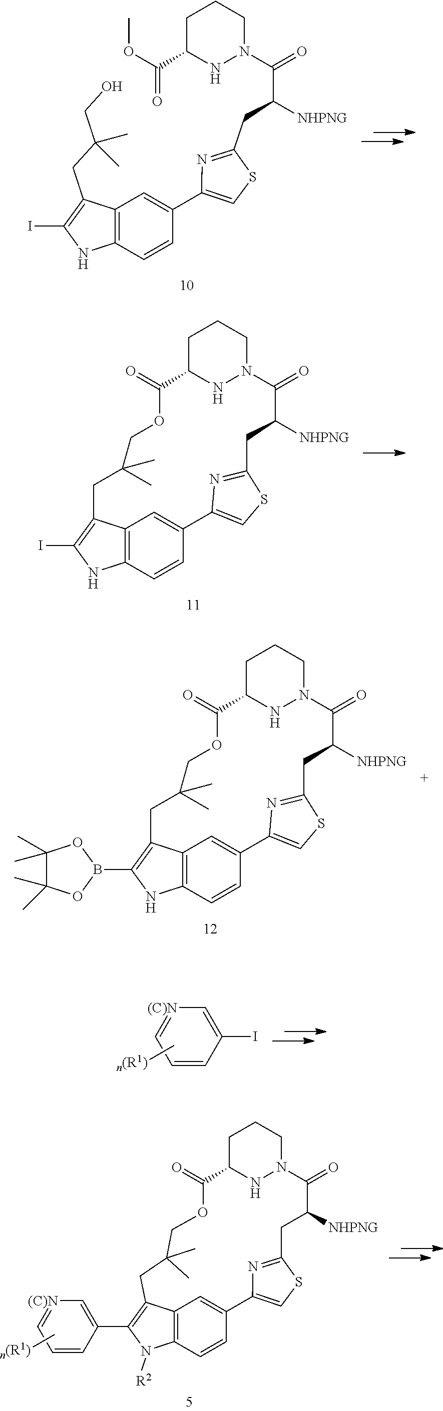

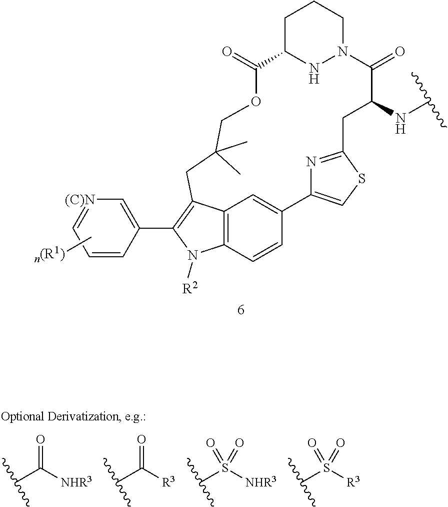

Alternatively, macrocyclic esters can be prepared as described in Scheme D2. An appropriately substituted and protected indolyl boronic ester (7) can be coupled in the presence of Pd catalyst with (S)-2-amino-3-(4-bromothiazol-2-yl)propanoic acid, followed by iodination, deprotection, and ester hydrolysis. Subsequent coupling with methyl (S)-hexahydropyridazine-3-carboxylate, followed by hydrolysis and macrolactonization can result in iodo intermediate (11). Subsequent palladium mediated borylation and coupling in the presence of Pd catalyst with an appropriately substituted iodo aryl or iodo heteroaryl intermediate can yield an appropriately protected macrocyclic intermediate. Alkylation, deprotection and coupling with an appropriately substituted carboxylic acid carboxylic acid (or other coupling partner) results in a macrocyclic product. Additional deprotection or functionalization steps could be required to produce a final compound 6.

Further, with respect to Scheme D2, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

Compounds of Table D1a or Table D1 b herein were prepared using methods disclosed in WO 2022/060836 or were prepared using methods described herein combined with the knowledge of one of skill in the art.

In some embodiments, the RAS(ON) inhibitor is a compound, or a pharmaceutically acceptable salt thereof, having the structure of Formula EI:

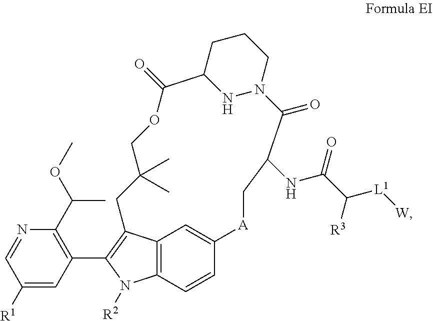

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
L¹ is absent or a linker;
W is a cross-linking group comprising a vinyl ketone, vinyl sulfone, ynone, or an alkynyl sulfone;
R¹ is hydrogen, optionally substituted 3 to 10-membered heterocycloalkyl, or optionally substituted C₁-C₆ heteroalkyl;
R² is optionally substituted C₁-C₆ alkyl; and
R³ is optionally substituted C₁-C₆ alkyl or optionally substituted C₁-C₃ heteroalkyl.

In some embodiments, W is a cross-linking group comprising a vinyl ketone, vinyl sulfone, or an ynone.

In some embodiments, provided herein is a compound, or pharmaceutically acceptable salt thereof, having the structure of Formula Ela:

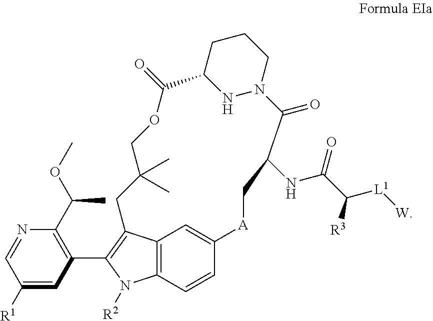

In some embodiments of Formula EI and subformula thereof, A is optionally substituted thiazole, optionally substituted oxazole, optionally substituted morpholino, optionally substituted pyrrolidinyl, optionally substituted pyridyl, optionally substituted azetidinyl, optionally substituted pyrazinyl, optionally substituted pyrimidine, optionally substituted piperidinyl, optionally substituted oxadiazole, optionally substituted thiadiazole, optionally substituted triazole, optionally substituted thiomorpholino, or optionally substituted phenyl.

In some embodiments, the RAS(ON) inhibitor is a compound, or pharmaceutically acceptable salt thereof, of structural Formula EII-1:

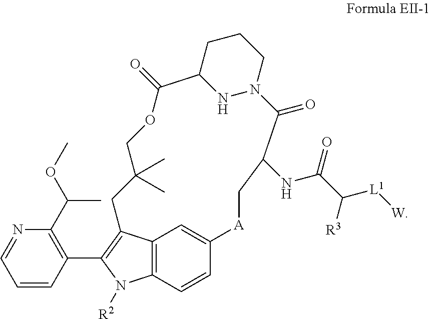

In some embodiments, the RAS(ON) inhibitor is a compound having the structure of Formula EII-2 is provided, or a pharmaceutically acceptable salt thereof:

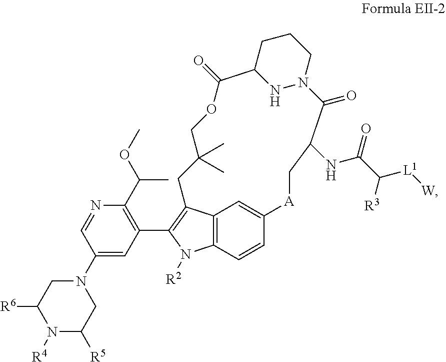

wherein R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.

In some embodiments, the RAS(ON) inhibitor has the structure of Formula EII-3, or a pharmaceutically acceptable salt thereof:

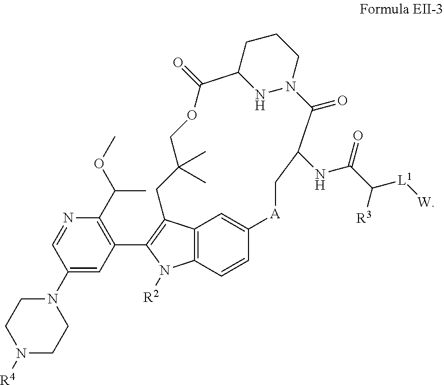

In some embodiments, the RAS(ON) inhibitor has the structure of Formula EII-4, or a pharmaceutically acceptable salt thereof:

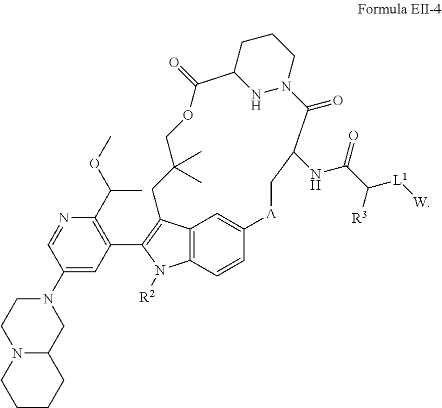

In some embodiments of Formula EI and subformula thereof, R² is:

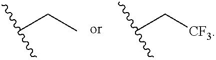

In some embodiments of Formula EI and subformula thereof, R³ is optionally substituted C₁-C₃ alkyl. In some embodiments, R³ is:

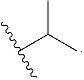 [SMILES: *C(C)C]

In some embodiments of Formula EI and subformula thereof, R³ is optionally substituted C₁-C₃ heteroalkyl. In some embodiments, R³ is:

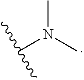 [SMILES: *N(C)C]

In some embodiments of Formula EI and subformula thereof, A is optionally substituted 5 to 10-membered heteroarylene. In some embodiments, A is:

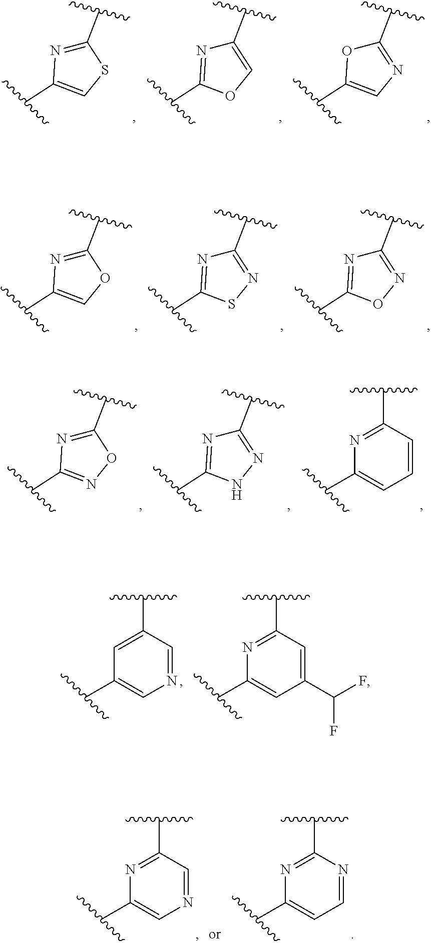

In some embodiments of Formula EI and subformula thereof, A is optionally substituted phenyl. In some embodiments, A is:

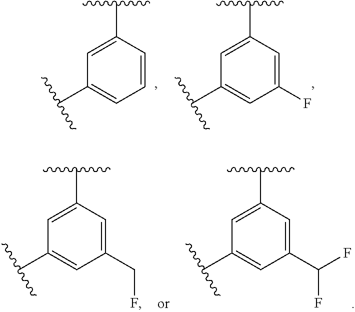 [SMILES: *c1cccc(*)c1 ; *c1cc(*)cc(F)c1 ; *c1cc(*)cc(CF)c1 ; *c1cc(*)cc(C(F)F)c1]

In some embodiments of Formula EI and subformula thereof, A is optionally substituted 3 to 6-membered heterocycloalkylene. In some embodiments, A is selected from the following, or a stereoisomer thereof:

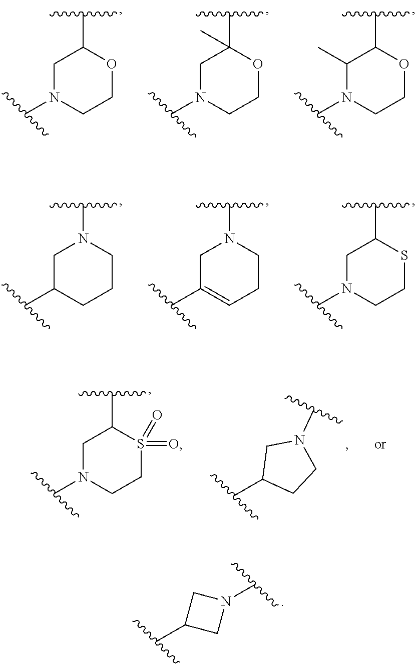

In some embodiments of Formula EI and subformula thereof, the linker is the structure of Formula III:

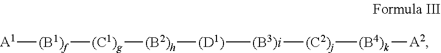

wherein A¹ is a bond between the linker and CH(R³); A² is a bond between Wand the linker; B¹, B², B³, and B⁴ each, independently, is selected from optionally substituted C₁-C₂ alkylene, optionally substituted C₁-C₃ heteroalkylene, O, S, and NR^{N}; each R^{N} is, independently, hydrogen, optionally substituted C₁-C₄ alkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C₁ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A².

In some embodiments of Formula EI and subformula thereof, the linker is or comprises a cyclic moiety. In some embodiments, the linker has the structure of Formula IIIa:

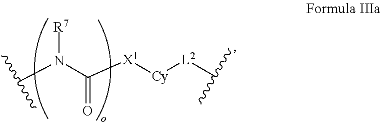

wherein o is 0 or 1;
R⁷ is hydrogen, optionally substituted C₁-C₆ alkyl, optionally substituted 3 to 8-membered cycloalkylene, or optionally substituted 3 to 8-membered heterocycloalkylene;
X¹ is absent, optionally substituted C₁-C₄ alkylene, 0, NCH₃, or optionally substituted C₁-C₄ heteroalkylene;
Cy is optionally substituted 3 to 8-membered cycloalkylene, optionally substituted 3 to 12-membered heterocycloalkylene, optionally substituted 6-10 membered arylene, or optionally substituted 5 to 10-membered heteroarylene; and
L² is absent, —SO₂—, —NH—, optionally substituted C₁-C₄ alkylene, optionally substituted C₁-C₄ heteroalkylene, or optionally substituted 3 to 6-membered heterocycloalkylene.

In some embodiments of Formula EI and subformula thereof, the linker is selected from, or a stereoisomer thereof:

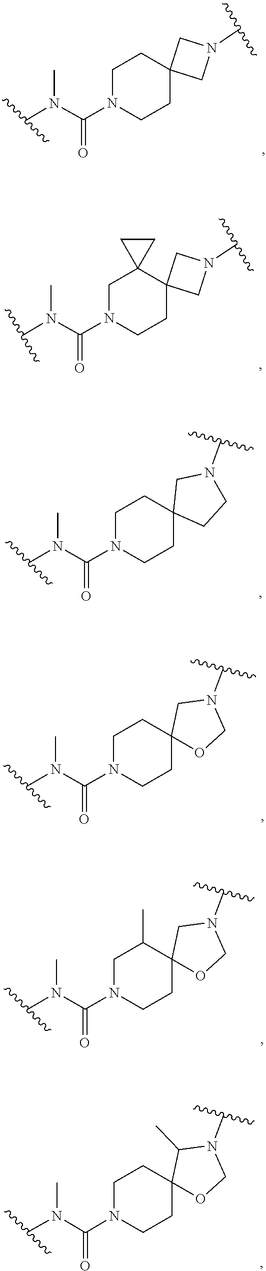 [SMILES: *N1CC2(CCN(C(=O)N(*)C)CC2)C1 ; *N1CC2(CCN(C(=O)N(*)C)CC23CC3)C1 ; *N1CCC2(CCN(C(=O)N(*)C)CC2)C1 ; *N1COC2(CCN(C(=O)N(*)C)CC2)C1 ; *N1COC2(CCN(C(=O)N(*)C)CC2C)C1 ; *N(C)C(=O)N1CCC2(CC1)OCN(*)C2C]

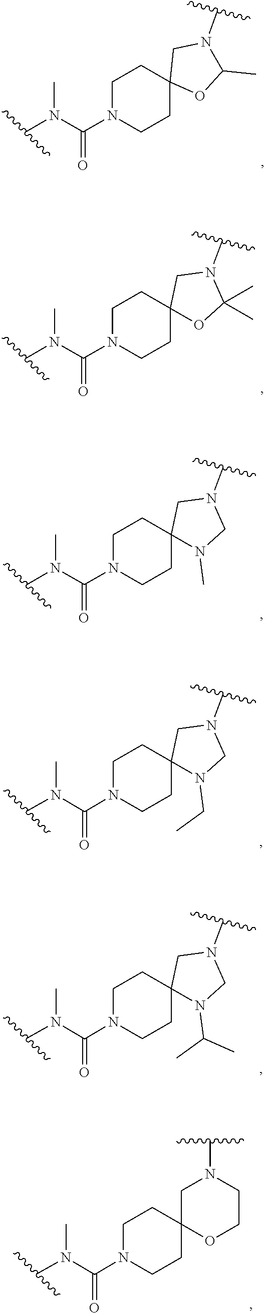 [SMILES: *N(C)C(=O)N1CCC2(CC1)CN(*)C(C)O2 ; *N(C)C(=O)N1CCC2(CC1)CN(*)C(C)(C)O2 ; *N1CN(C)C2(CCN(C(=O)N(*)C)CC2)C1 ; *N1CN(CC)C2(CCN(C(=O)N(*)C)CC2)C1 ; *N1CN(C(C)C)C2(CCN(C(=O)N(*)C)CC2)C1 ; *N1CCOC2(CCN(C(=O)N(*)C)CC2)C1]

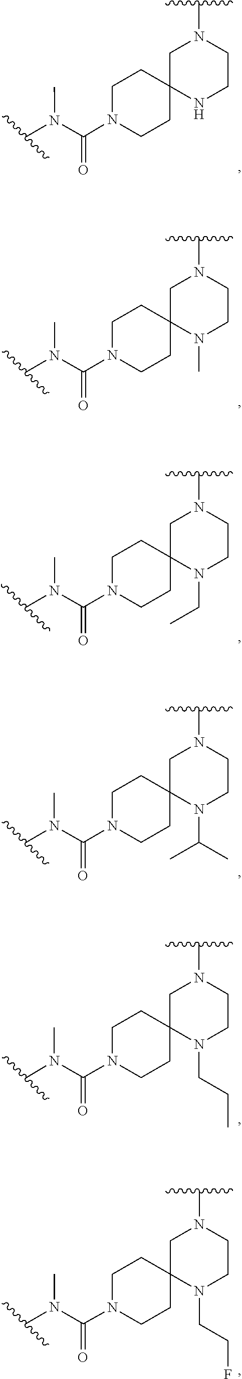 [SMILES: *N1CCNC2(CCN(C(=O)N(*)C)CC2)C1 ; *N1CCN(C)C2(CCN(C(=O)N(*)C)CC2)C1 ; *N1CCN(CC)C2(CCN(C(=O)N(*)C)CC2)C1 ; *N1CCN(C(C)C)C2(CCN(C(=O)N(*)C)CC2)C1 ; *N1CCN(CCC)C2(CCN(C(=O)N(*)C)CC2)C1 ; *N1CCN(CCF)C2(CCN(C(=O)N(*)C)CC2)C1]

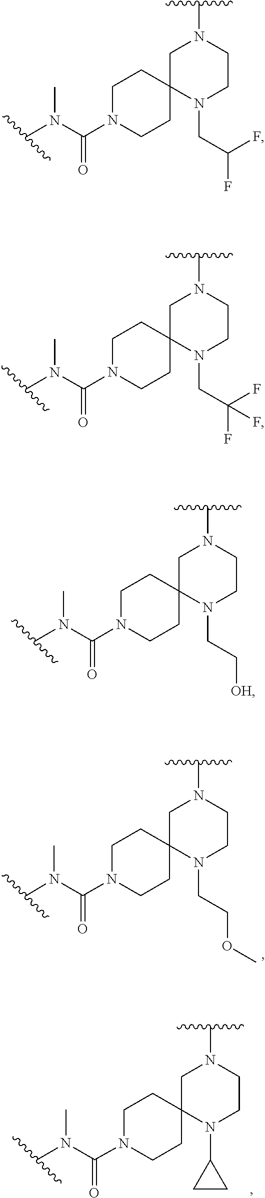 [SMILES: *N1CCN(CC(F)F)C2(CCN(C(=O)N(*)C)CC2)C1 ; *N1CCN(CC(F)(F)F)C2(CCN(C(=O)N(*)C)CC2)C1 ; *N1CCN(CCO)C2(CCN(C(=O)N(*)C)CC2)C1 ; *N1CCN(CCOC)C2(CCN(C(=O)N(*)C)CC2)C1 ; *N1CCN(C2CC2)C2(CCN(C(=O)N(*)C)CC2)C1]

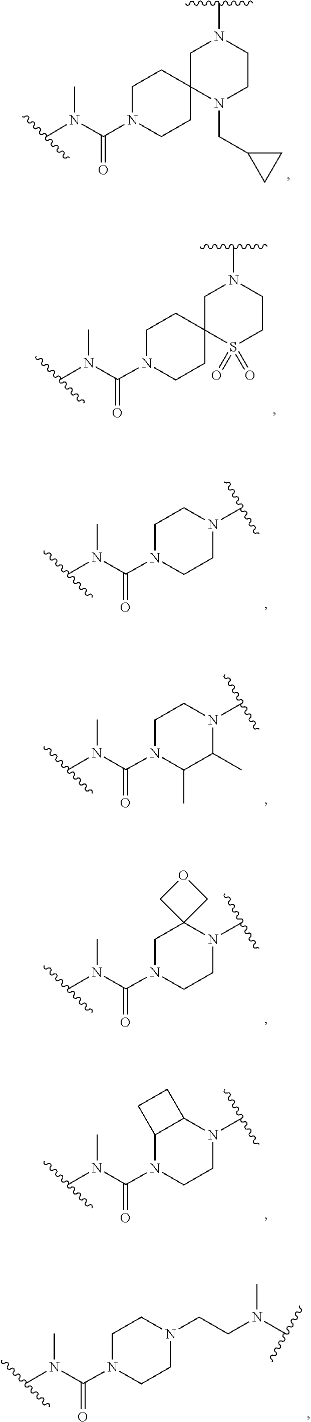

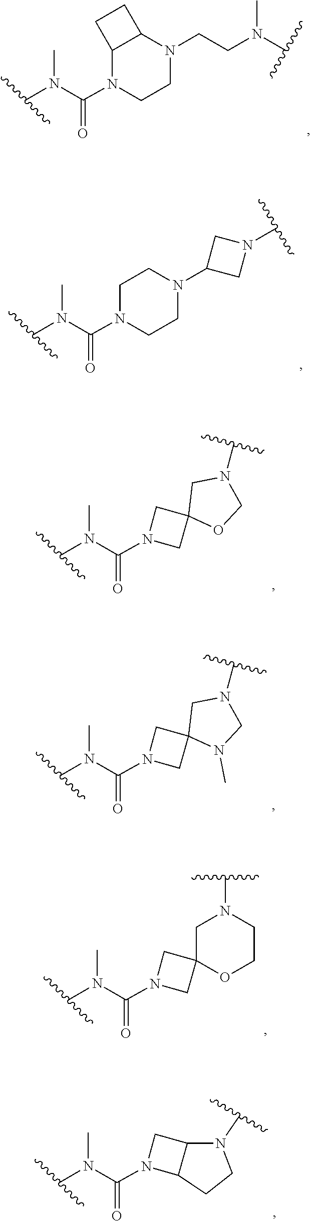 [SMILES: *N(C)CCN1CCN(C(=O)N(*)C)C2CCC21 ; *N1CC(N2CCN(C(=O)N(*)C)CC2)C1 ; *N1COC2(C1)CN(C(=O)N(*)C)C2 ; *N1CN(C)C2(C1)CN(C(=O)N(*)C)C2 ; *N1CCOC2(C1)CN(C(=O)N(*)C)C2 ; *N(C)C(=O)N1CC2C1CCN2*]

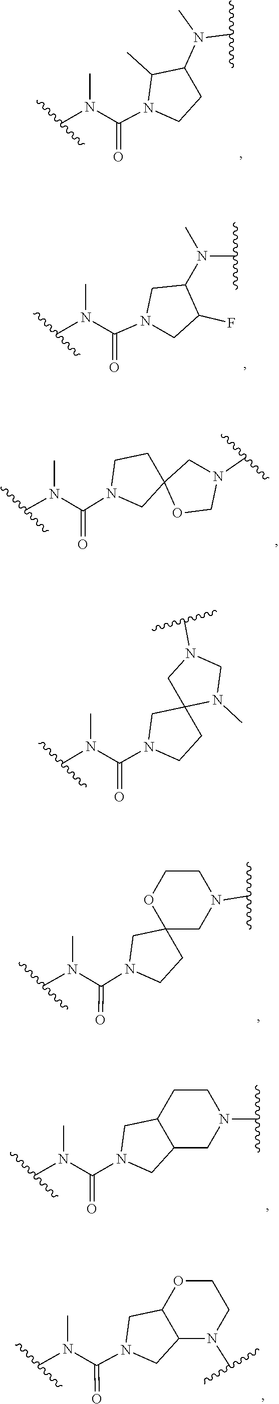

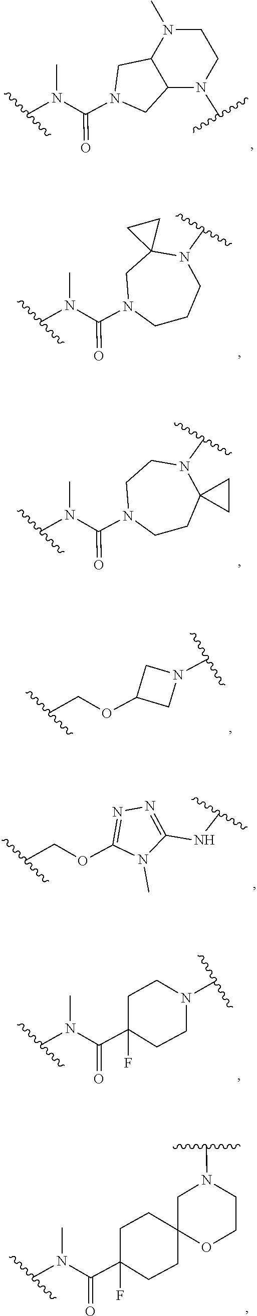

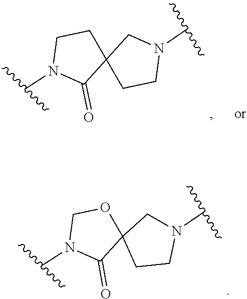 [SMILES: *N1CCC2(CCN(*)C2=O)C1 ; *N1CCC2(C1)OCN(*)C2=O]

In some embodiments, the RAS(ON) inhibitor has the structure of Formula EII-5, or a pharmaceutically acceptable salt thereof:

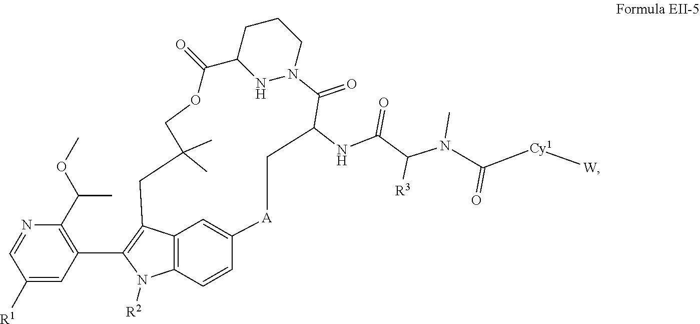

wherein Cy¹ is optionally substituted spirocyclic 8 to 11-membered heterocycloalkylene or optionally substituted bicyclic 7 to 9-membered heterocycloalkylene; and
wherein W comprises a vinyl ketone or a vinyl sulfone.

In some embodiments, Cy¹ is optionally substituted spirocyclic 10 to 11-membered heterocycloalkylene.

In some embodiments, the RAS(ON) inhibitor has the structure of Formula II-5a, or a pharmaceutically acceptable salt thereof:

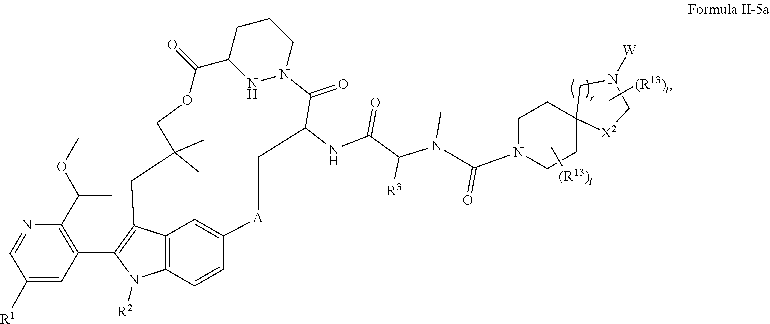

wherein X² is O, C(R¹¹)₂, NR¹², S, or SO₂,
r is 1 or 2;
each t is, independently, 0, 1, or 2;
R¹¹ and R¹² are each, independently, hydrogen, optionally substituted C₁-C₄ alkyl, optionally substituted C₂-C₄ heteroalkyl, or optionally substituted 3 to 5-membered cycloalkyl; and each R¹³ is, independently, —CH₃.

In some embodiments of Formula EI and subformula thereof, r is 1. In some embodiments, r is 2.

In some embodiments, X² is O. In some embodiments, X² is S. In some embodiments, X² is SO₂.

In some embodiments of Formula EI and subformula thereof, X² is NR¹². In some embodiments, R¹² is selected from, or a stereoisomer thereof:

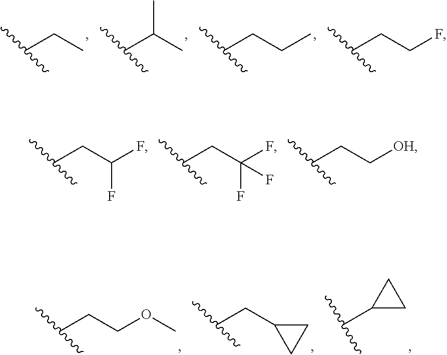

In some embodiments of Formula EI and subformula thereof, X² is C(R¹¹)₂. In some embodiments, each R¹¹ is hydrogen.

In some embodiments of Formula EI and subformula thereof, W is a cross-linking group comprising a vinyl ketone. In some embodiments, W has the structure of Formula IVa:

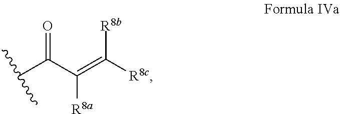

wherein R^{8a}, R^{8b}, and R^{8c} are, independently, hydrogen, —CN, halogen, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is selected from, or a stereoisomer thereof:

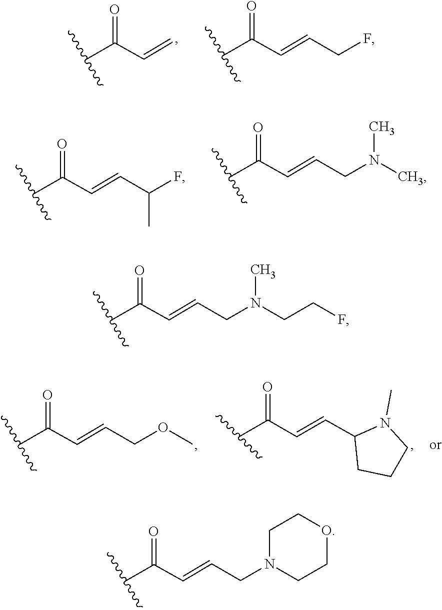

In some embodiments of Formula EI and subformula thereof, W is a cross-linking group comprising a vinyl sulfone. In some embodiments, W has the structure of Formula IVc:

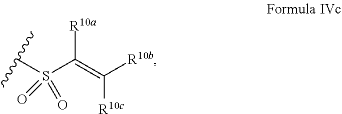

wherein R^{10a}, R^{10b}, and R^{10c} are, independently, hydrogen, —CN, or —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated heterocycloalkyl. In some embodiments, W is:

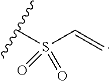 [SMILES: *S(=O)(=O)C=C]

In some embodiments of Formula EI and subformula thereof, W is a cross-linking group comprising an ynone. In some embodiments, W has the structure of Formula IVb:

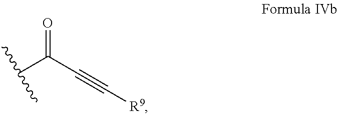

wherein R⁹ is hydrogen, —C₁-C₃ alkyl optionally substituted with one or more substituents independently selected from —OH, —O—C₁-C₃ alkyl, —NH₂, —NH(C₁-C₃ alkyl), —N(C₁-C₃ alkyl)₂, or a 4 to 7-membered saturated cycloalkyl, or a 4 to 7-membered saturated heterocycloalkyl. In some embodiments, W is selected from:

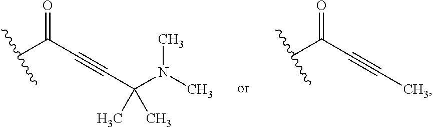 [SMILES: *C(=O)C#CC(C)(C)N(C)C ; "or" ; *C(=O)C#CC]

In some embodiments, the RAS(ON) inhibitor has the structure of Formula EII-6:

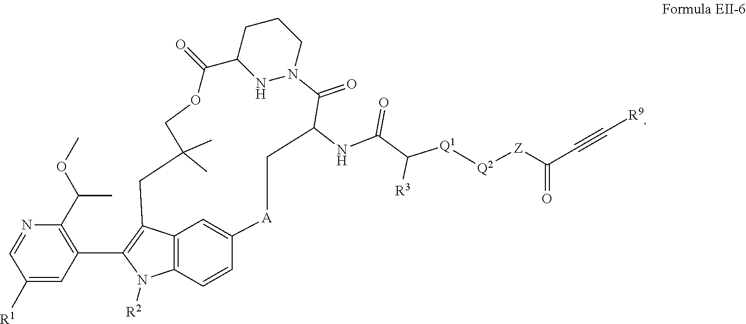

wherein Q¹ is CH₂, NR^{N}, or O;
Q² is CO, NR^{N}, or O; and
Z is optionally substituted 3 to 6-membered heterocycloalkylene or optionally substituted 5 to 10-membered heteroarylene; or
wherein Q¹-Q²-Z is an optionally substituted 9 to 10-membered spirocyclic heterocycloalkylene.

In some embodiments, the RAS(ON) inhibitor has the structure of Formula EII-6a:

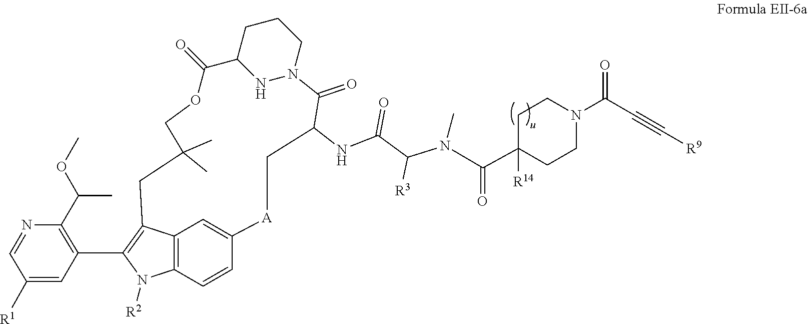

wherein R¹⁴ is fluoro, hydrogen, or C₁-C₃ alkyl; and
u is 0 or 1.

In some embodiments, R¹⁴ is fluoro and u is 1. In some embodiments, R¹⁴ is hydrogen and u is 0.

In some embodiments, the RAS(ON) inhibitor has the structure of Formula EII-6b:

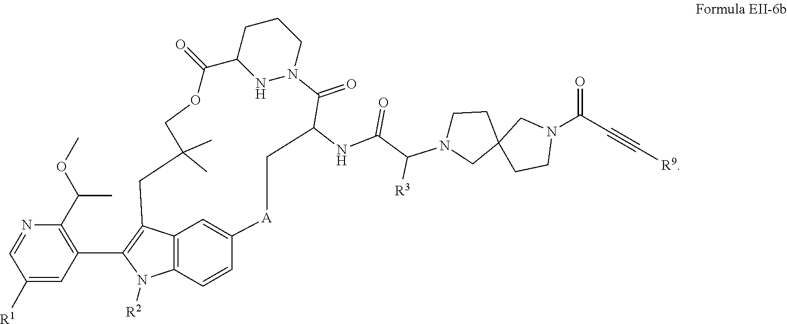

In some embodiments, a compound of the present invention has the structure of Formula EII-6c:

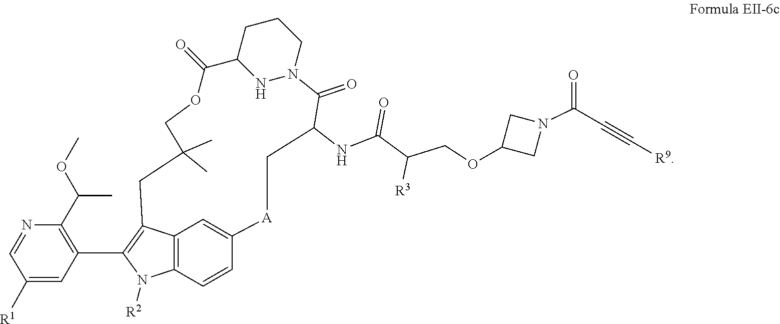

In some embodiments, a compound of the present invention is selected from Table E1, or a pharmaceutically acceptable salt or stereoisomer thereof. In some embodiments, a compound of the present invention is selected from Table E1, or a pharmaceutically acceptable salt or atropisomer thereof.

TABLE E1

| Ex# | Structure |
|---|---|
| EA1 | 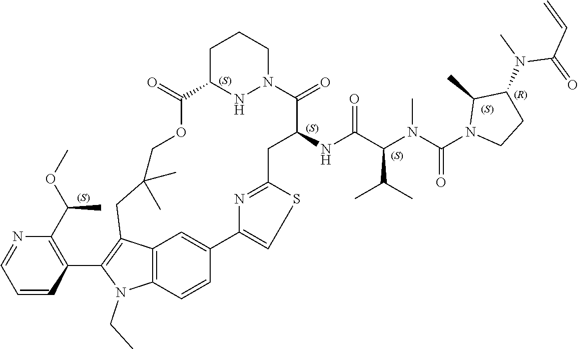 |
| EA2 | 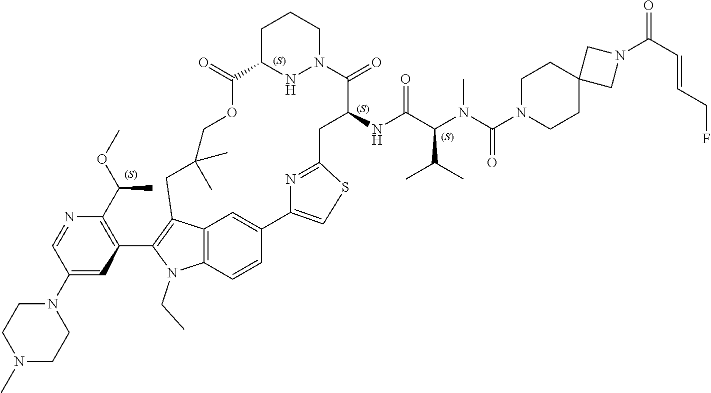 |
| EA3 | 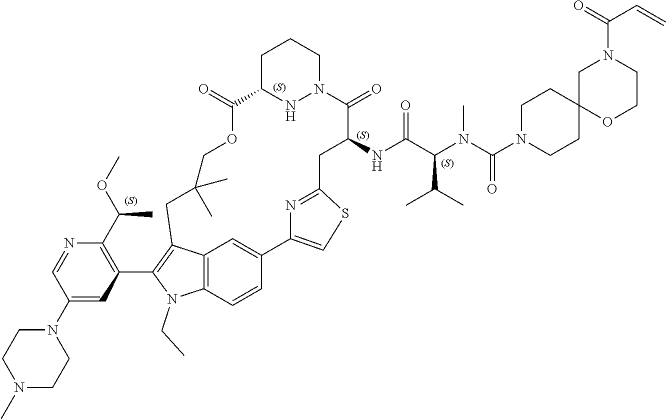 |
| EA4 | 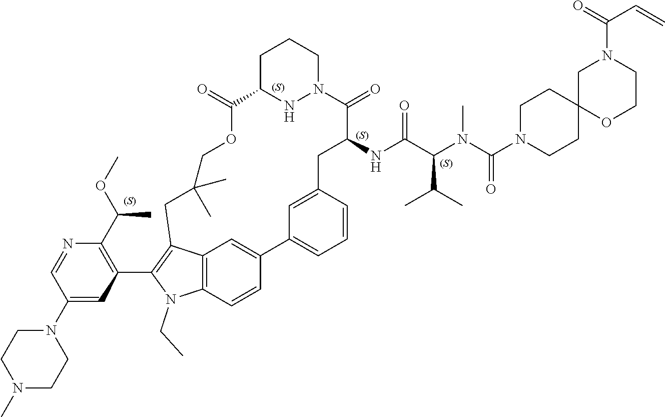 |
| EA5 | 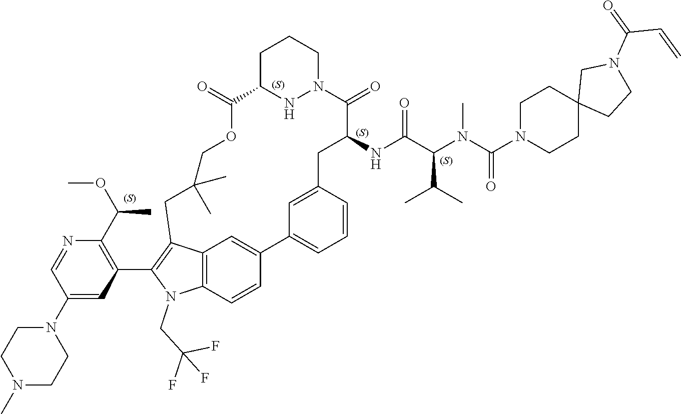 |
| EA6 | 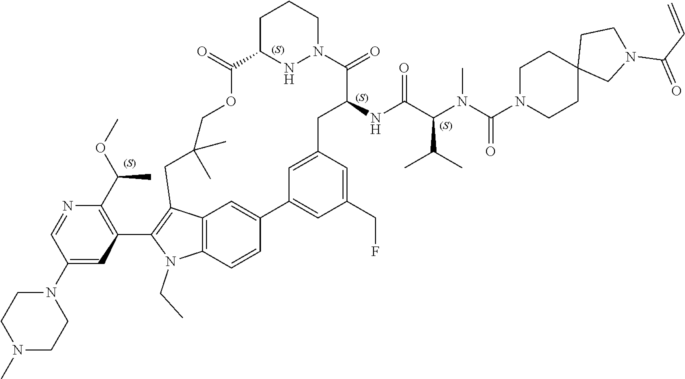 |
| EA7 | 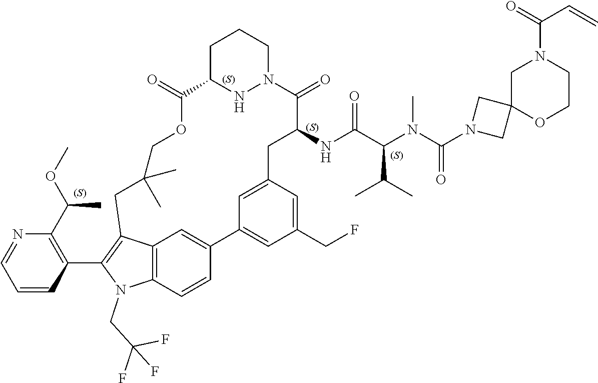 |
| EA8 | 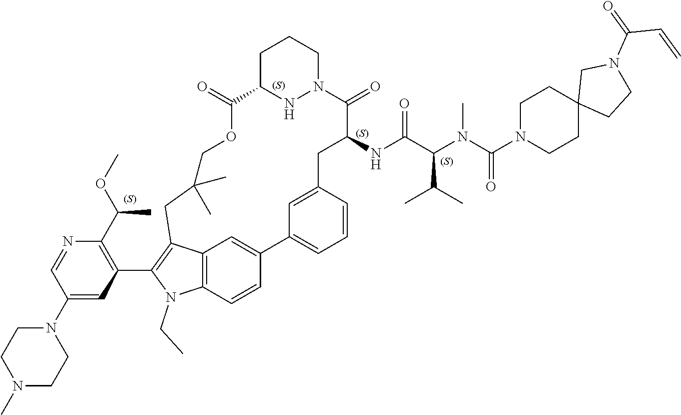 |
| EA9 | 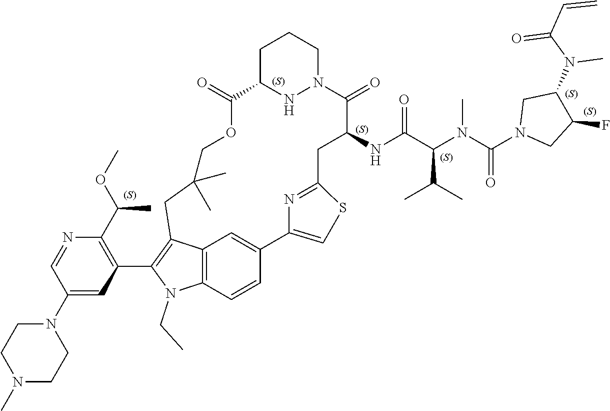 |
| EA10 | 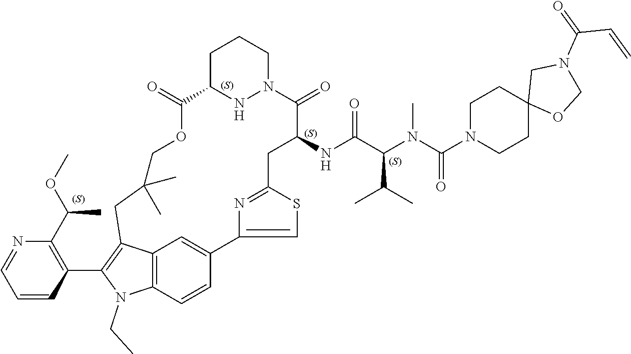 |
| EA11 | 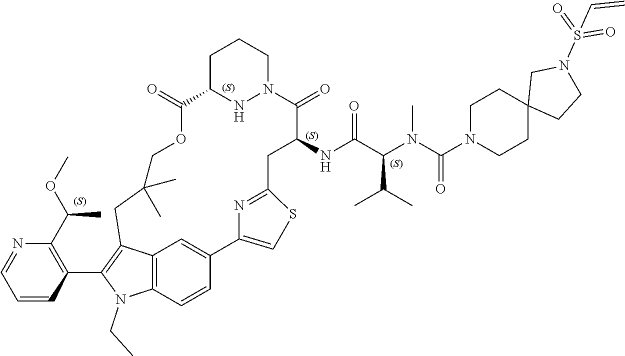 |
| EA12 | 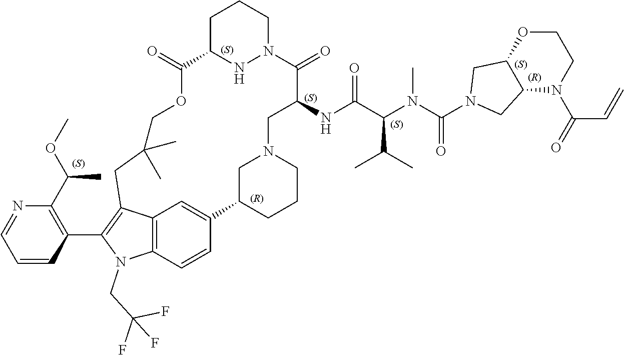 |
| EA13 | 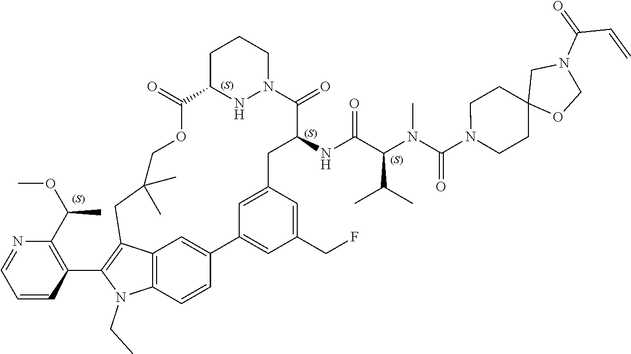 |
| EA14 | 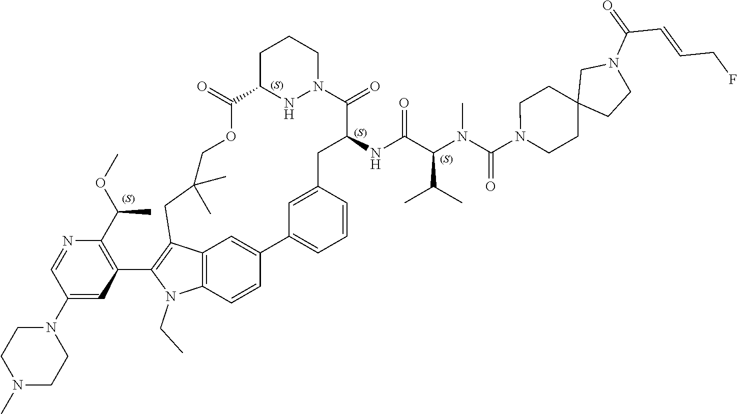 |
| EA15 | 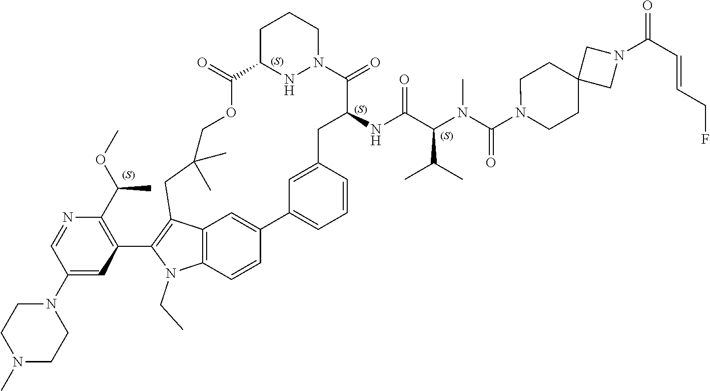 |
| EA16 | 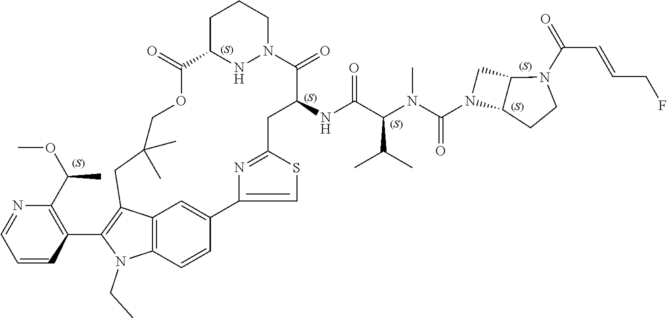 |
| EA17 | 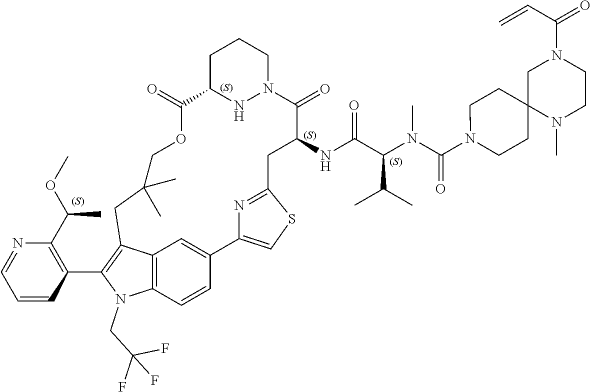 |
| EA18 | 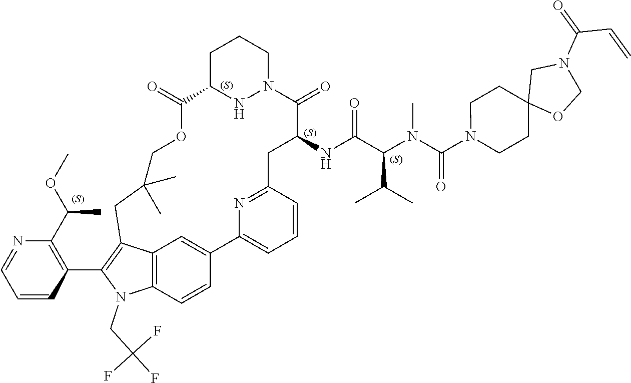 |
| EA19 | 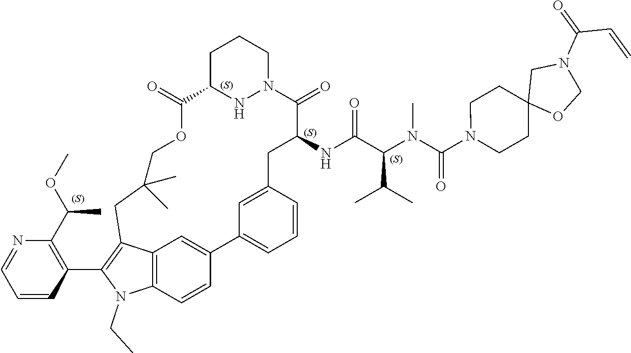 |
| EA20 | 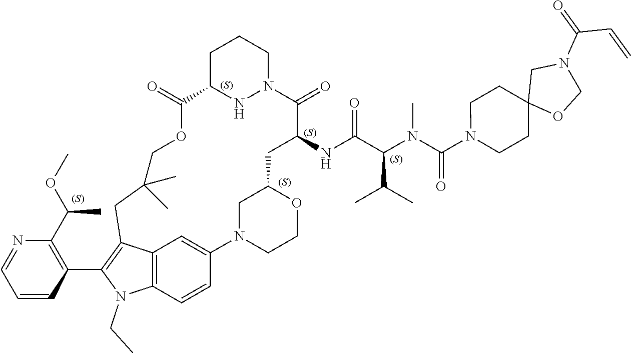 |
| EA21 | 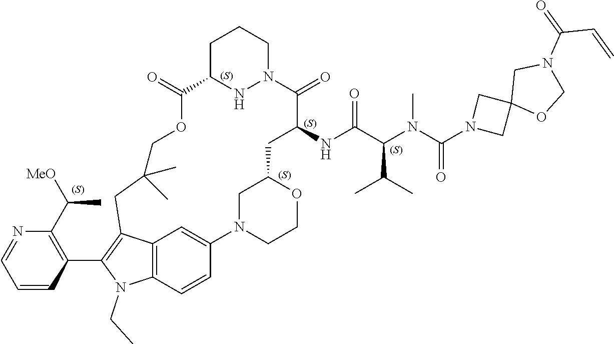 |
| EA22 | 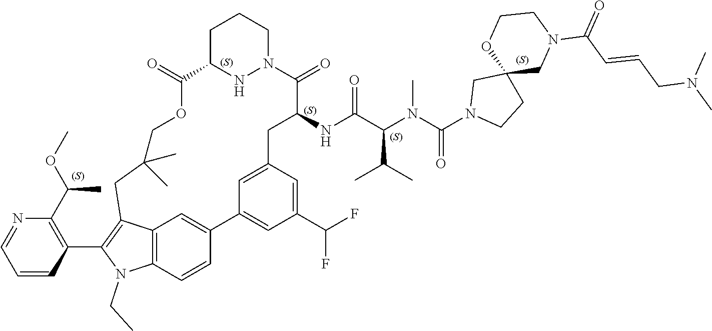 |
| EA23 | 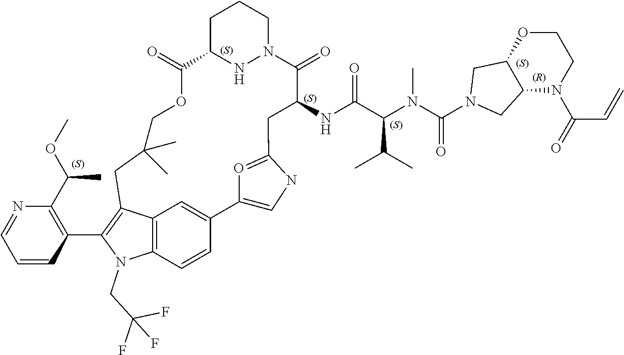 |
| EA24 | 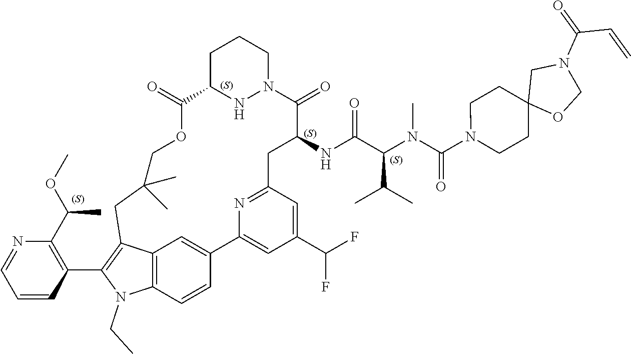 |
| EA25 | 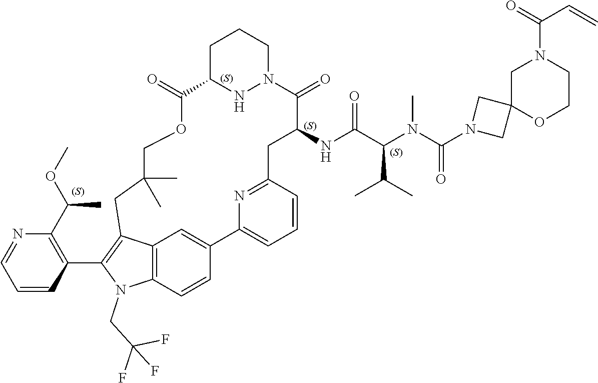 |
| EA26 | 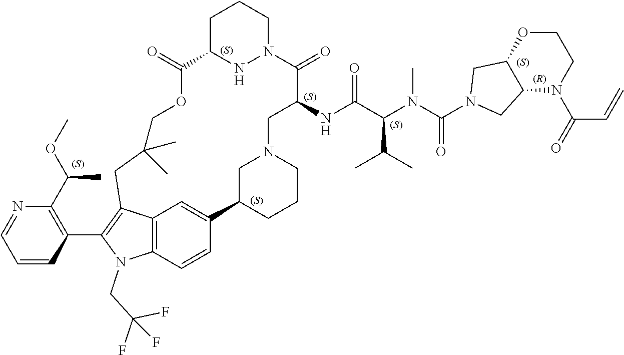 |
| EA27 | 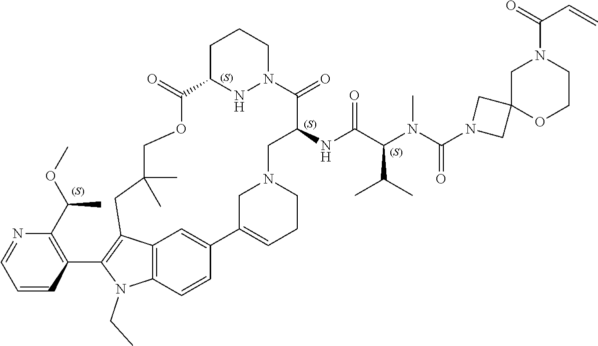 |
| EA28 | 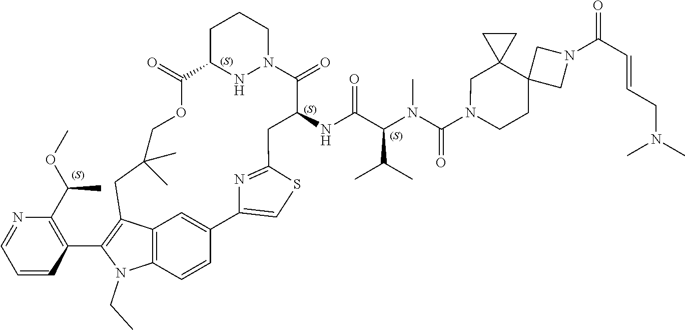 |
| EA29 | 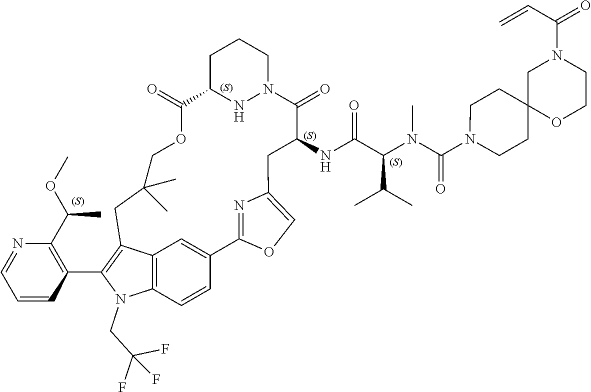 |
| EA30 | 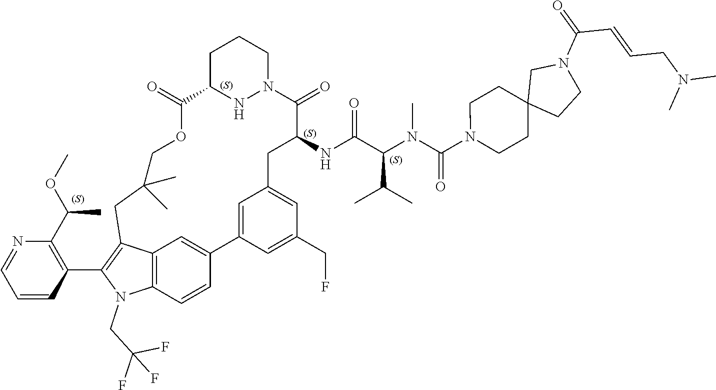 |
| EA31 | 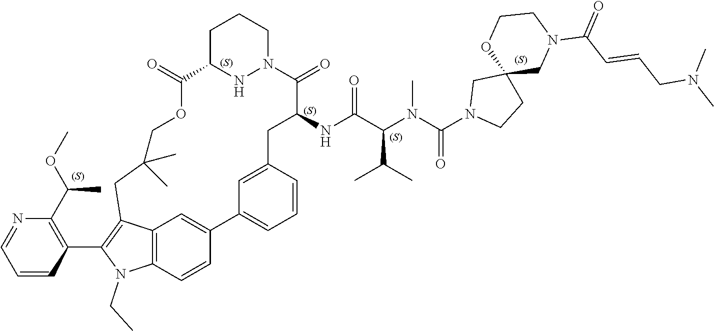 |
| EA32 | 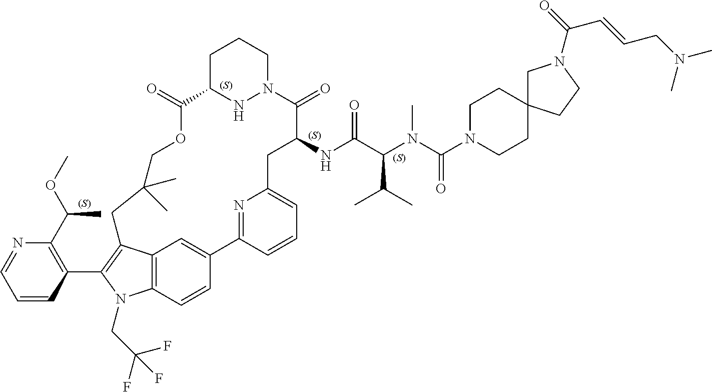 |
| EA33 | 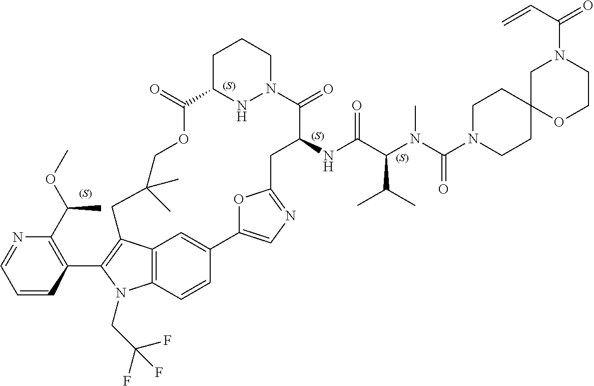 |
| EA34 | 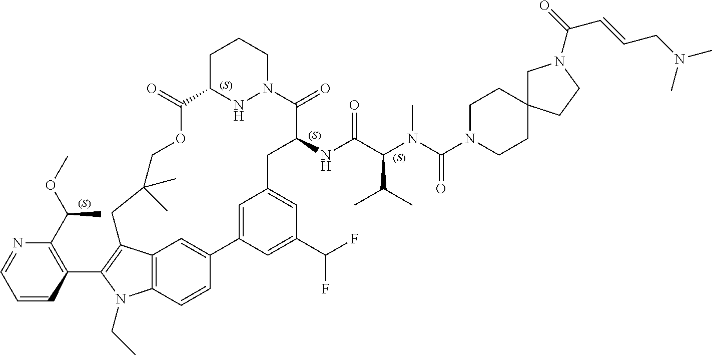 |
| EA35 | 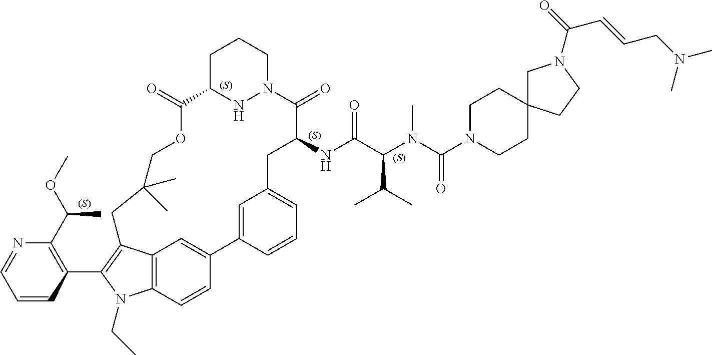 |
| EA36 | 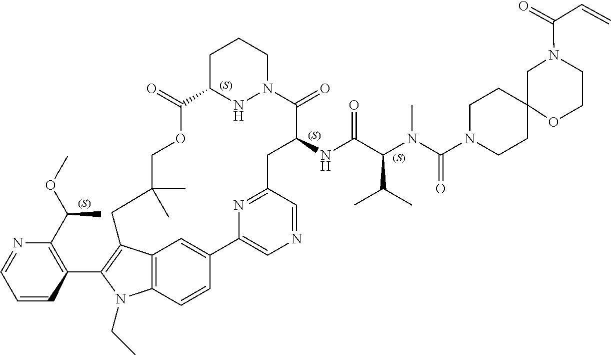 |
| EA37 | 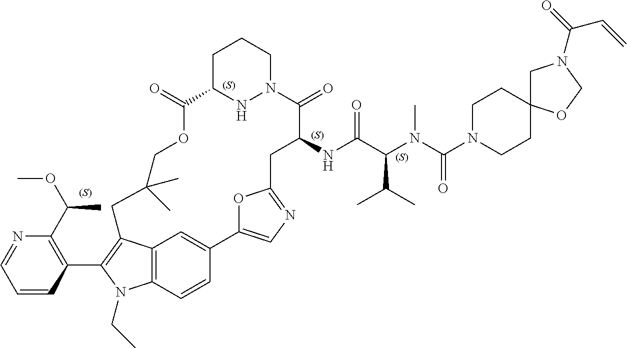 |
| EA38 | 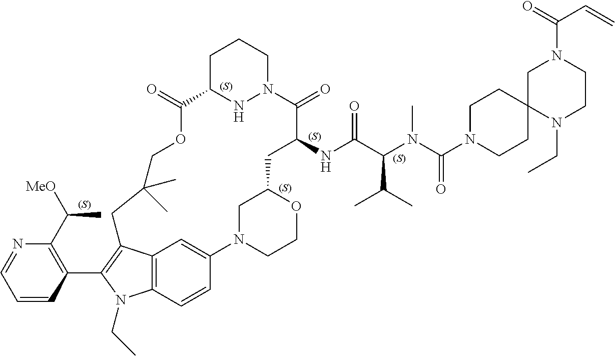 |
| EA39 | 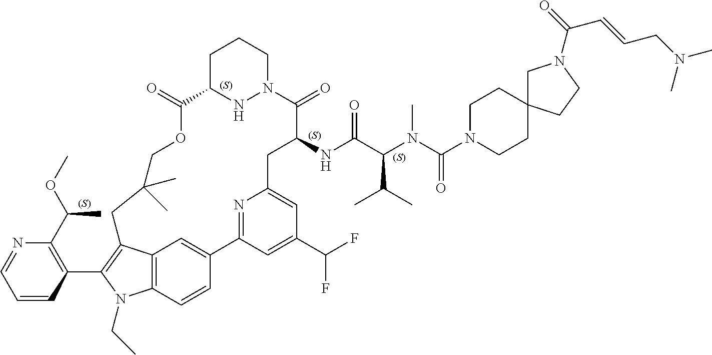 |
| EA40 | 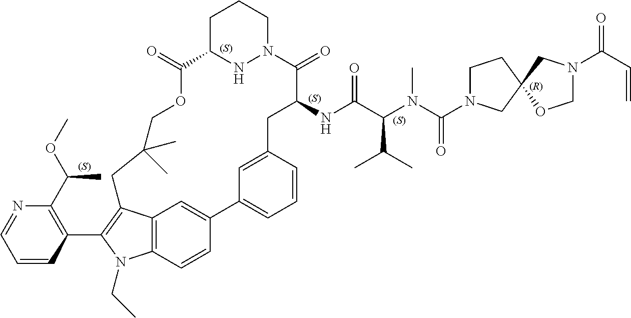 |
| EA41 | 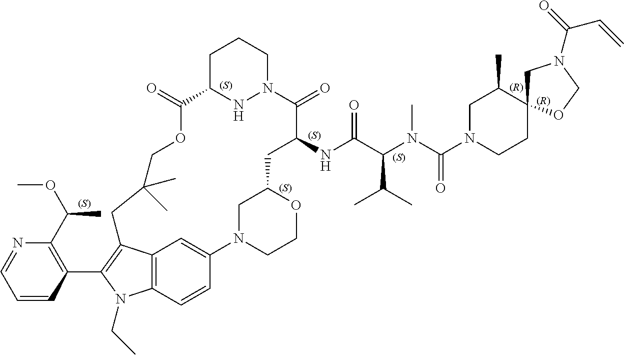 |
| EA42 | 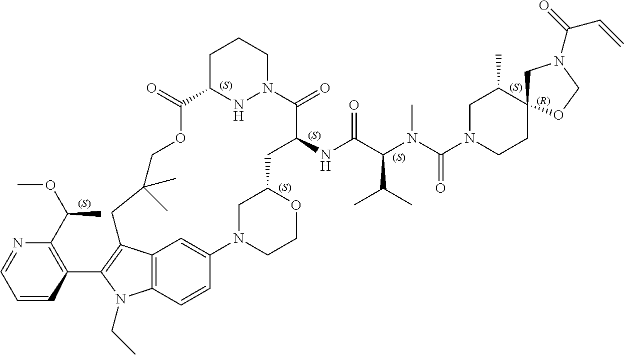 |
| EA43 | 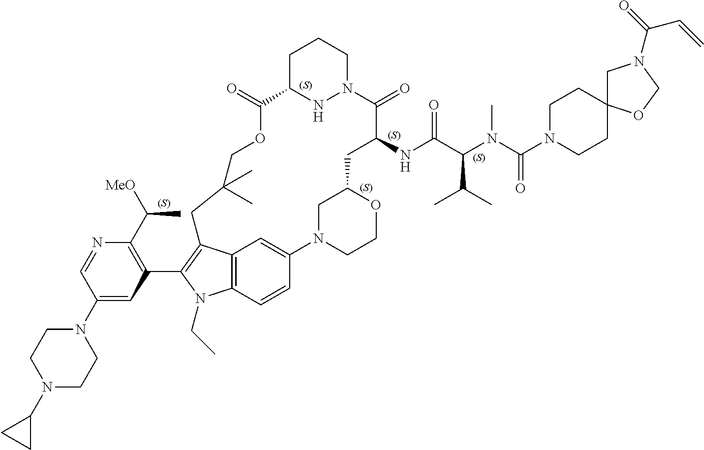 |
| EA44 | 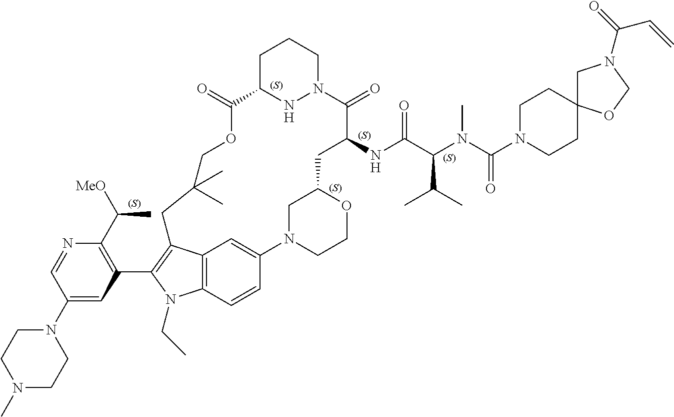 |
| EA45 | 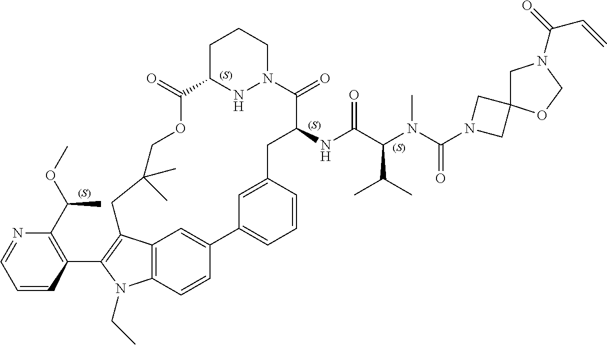 |
| EA46 | 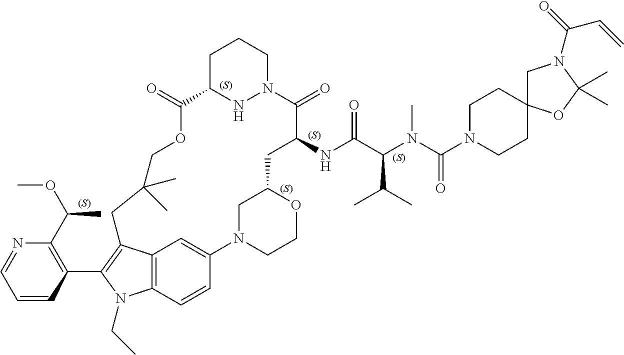 |
| EA47 | 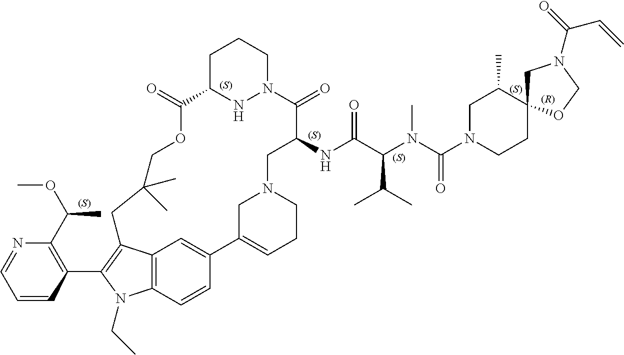 |
| EA48 | 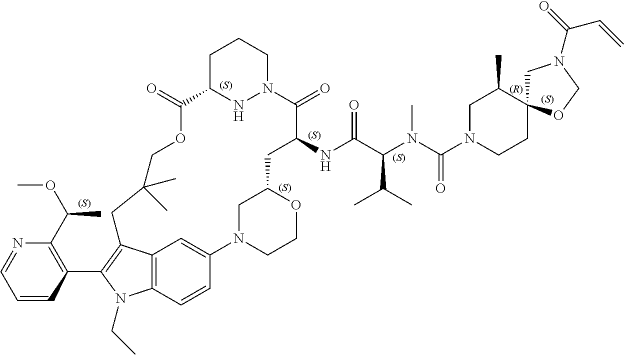 |
| EA49 | 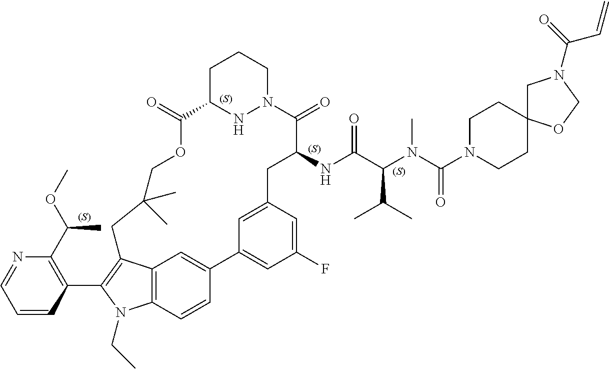 |
| EA50 | 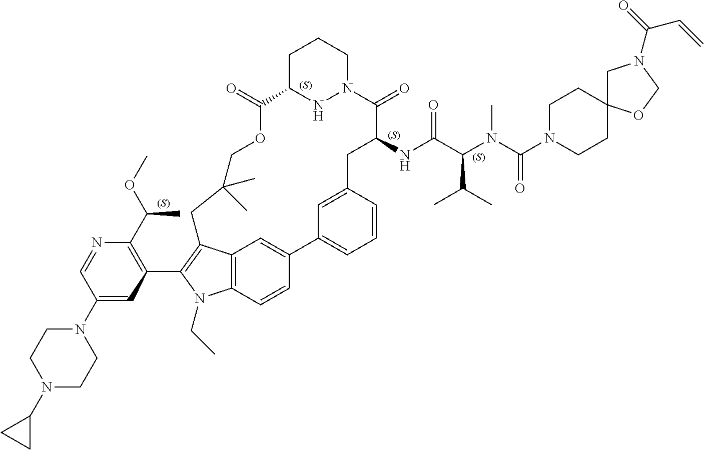 |
| EA51 | 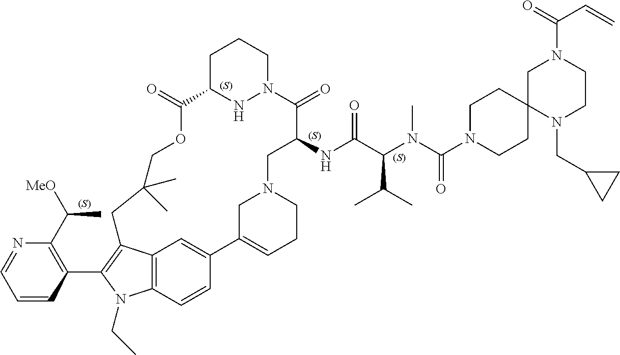 |
| EA52 | 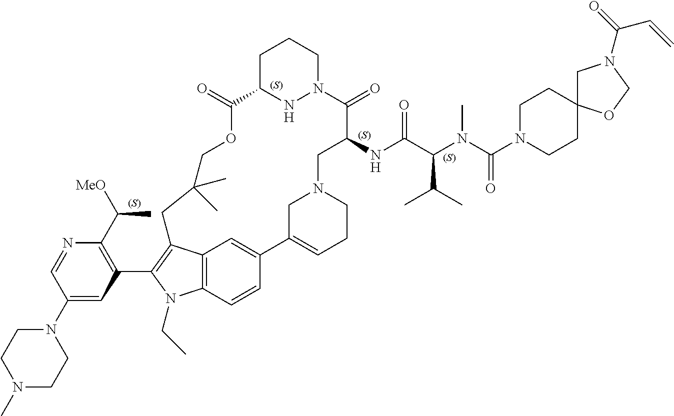 |
| EA53 | 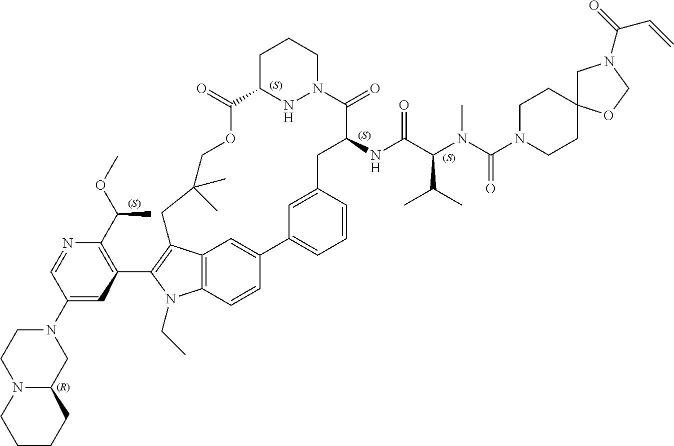 |
| EA54 | 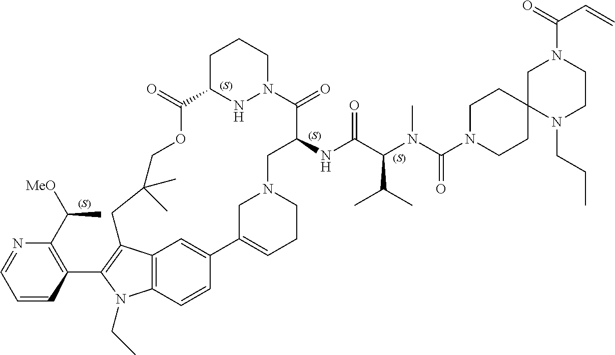 |
| EA55 | 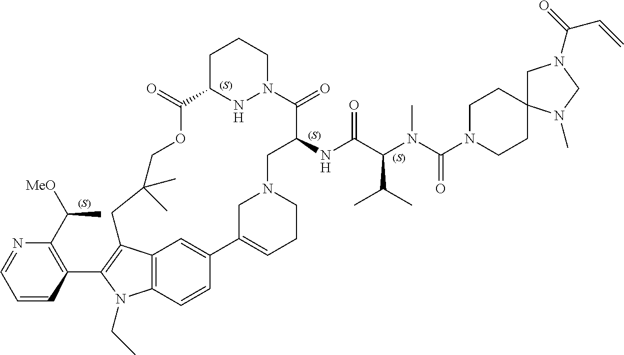 |
| EA56 | 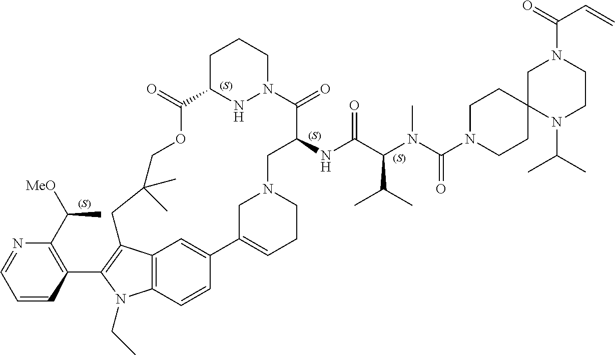 |
| EA57 | 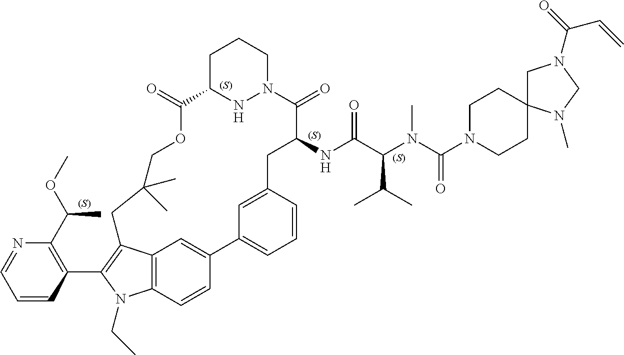 |
| EA58 | 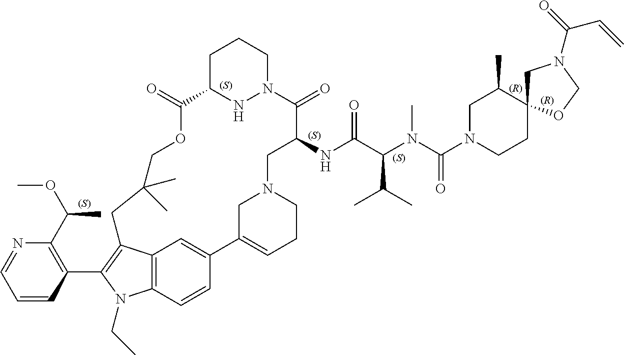 |
| EA59 | 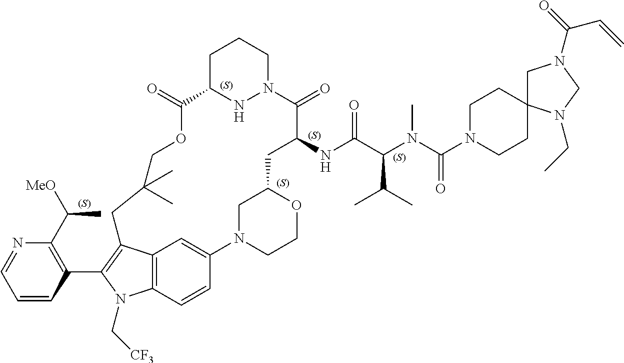 |
| EA60 | 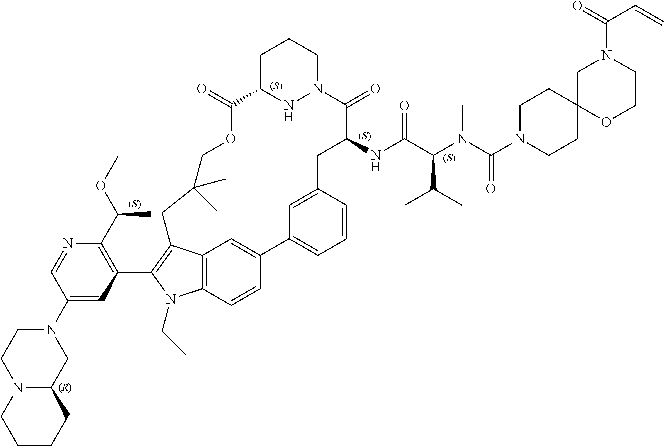 |
| EA61 | 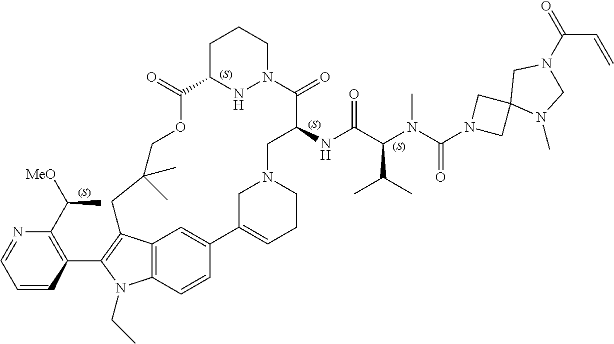 |
| EA62 | 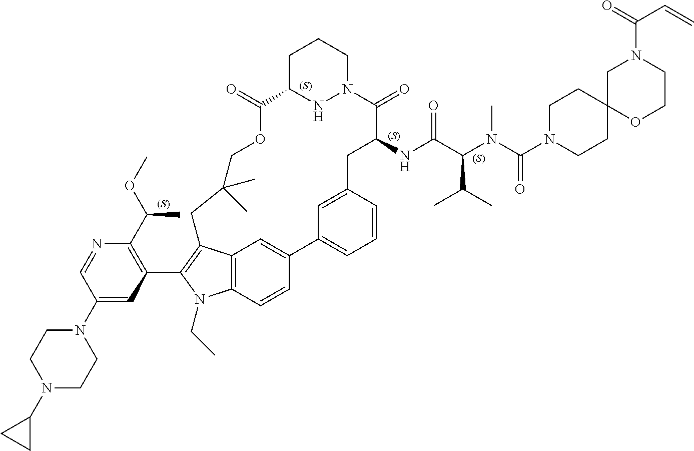 |
| EA63 | 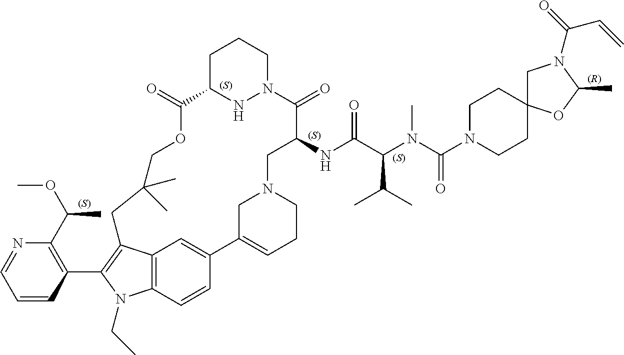 |
| EA64 | 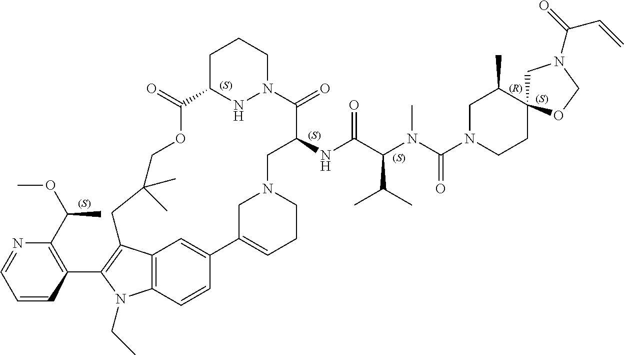 |
| EA65 | 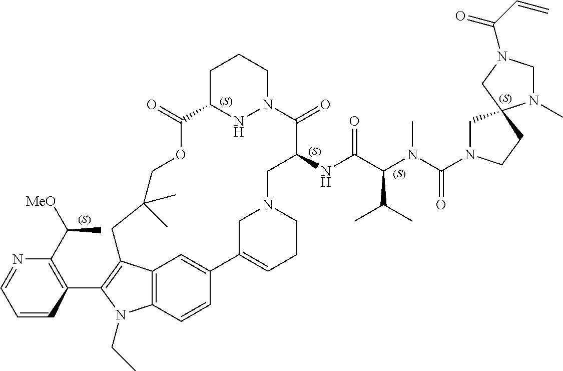 |
| EA66 | 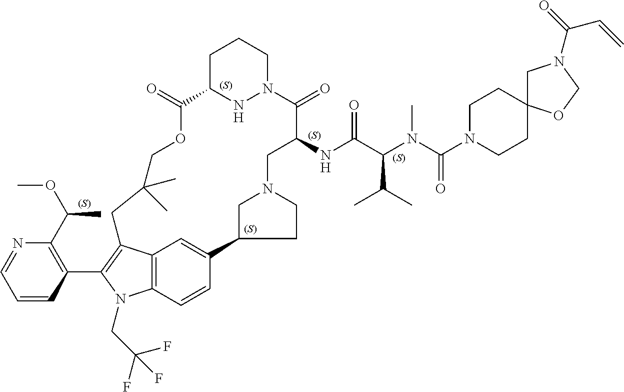 |
| EA67 | 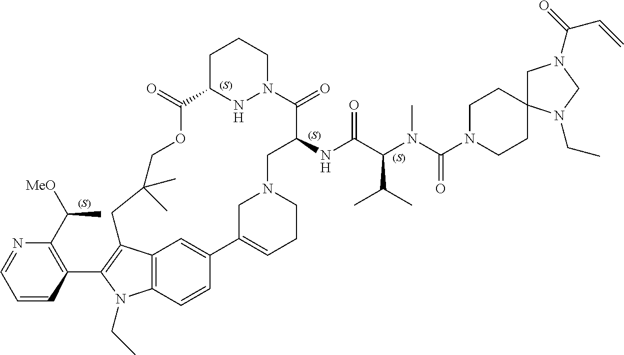 |
| EA68 | 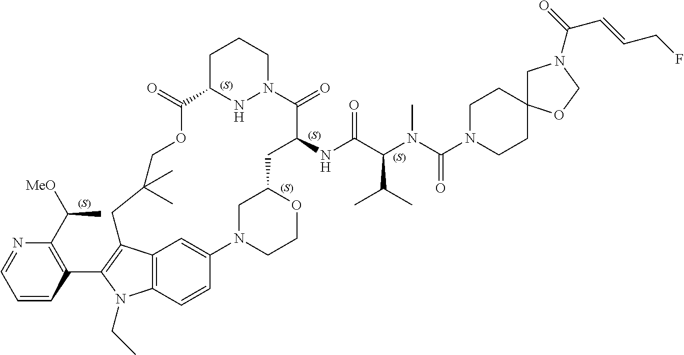 |
| EA69 | 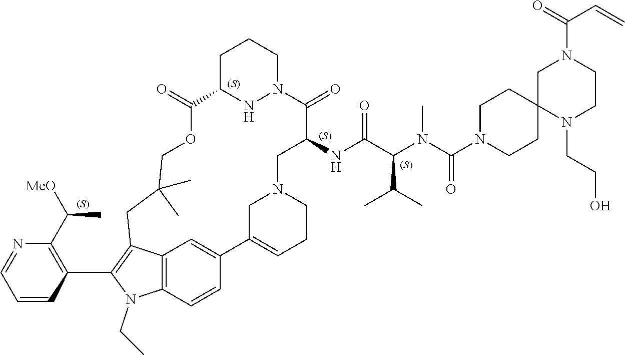 |
| EA70 | 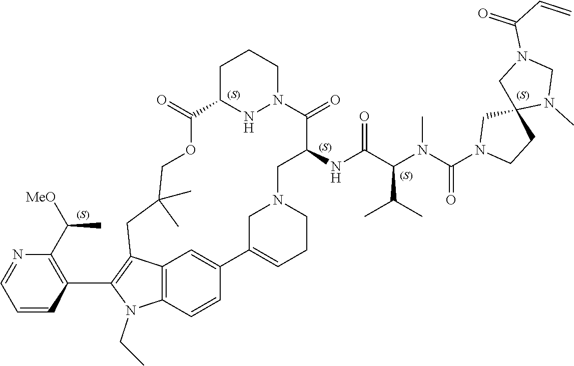 |
| EA71 | 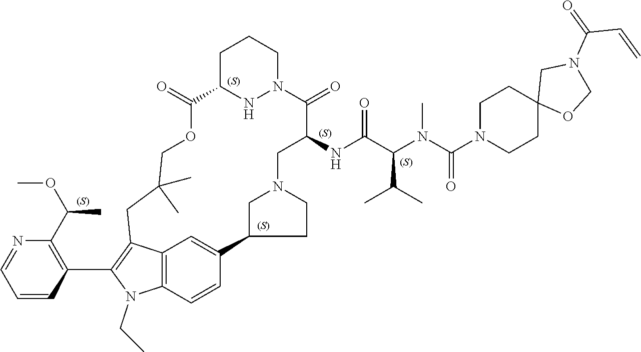 |
| EA72 | 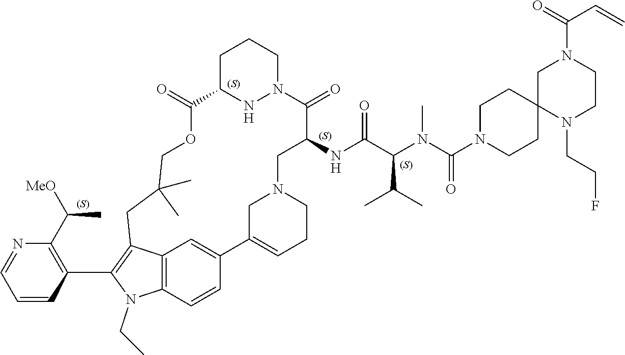 |
| EA73 | 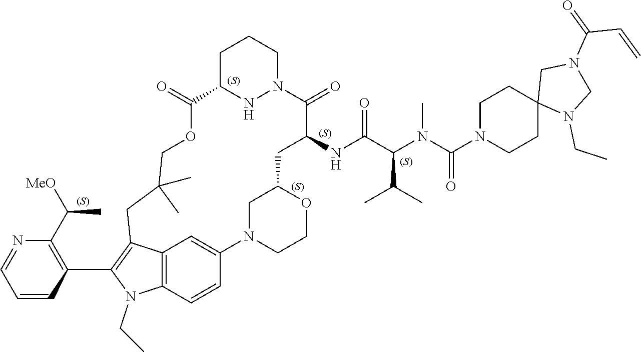 |
| EA74 | 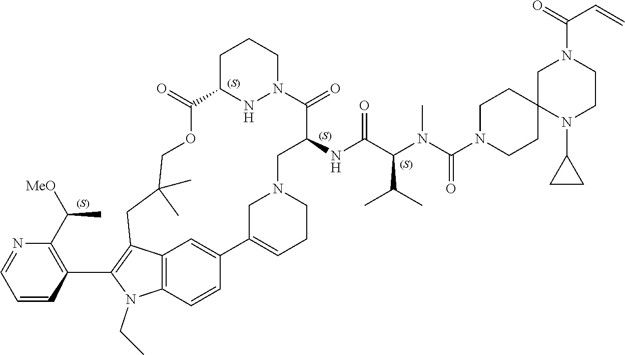 |
| EA75 | 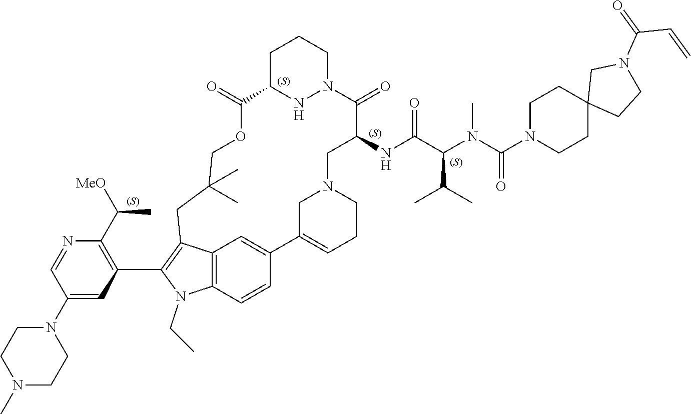 |
| EA76 | 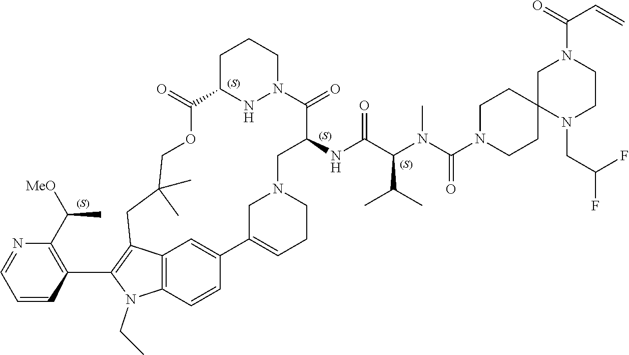 |
| EA77 | 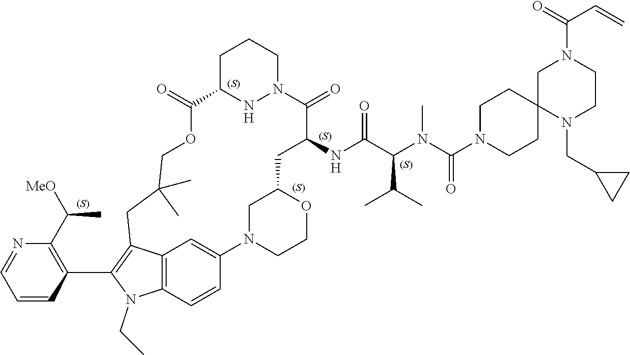 |
| EA78 | 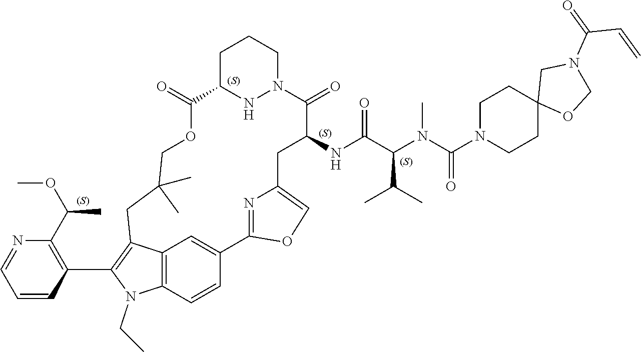 |
| EA79 | 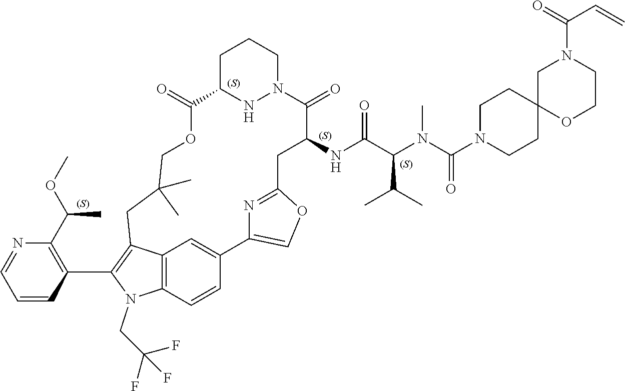 |
| EA80 | 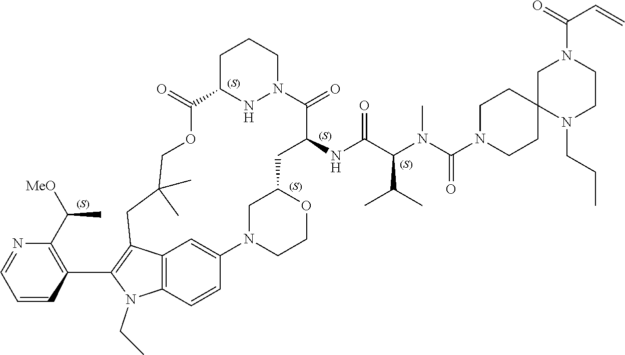 |
| EA81 | 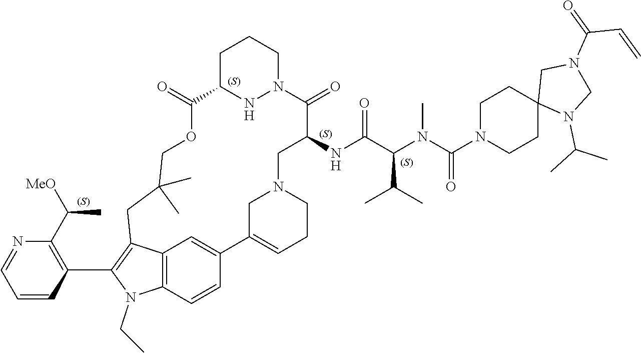 |
| EA82 | 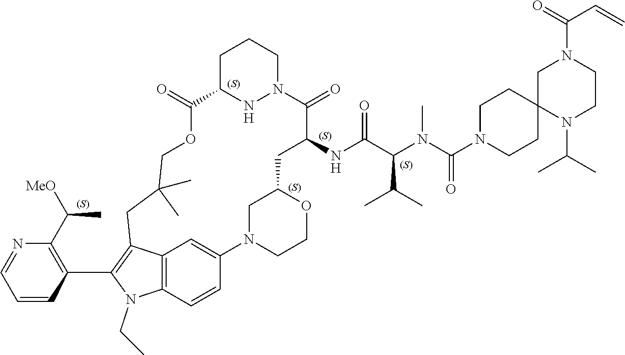 |
| EA83 | 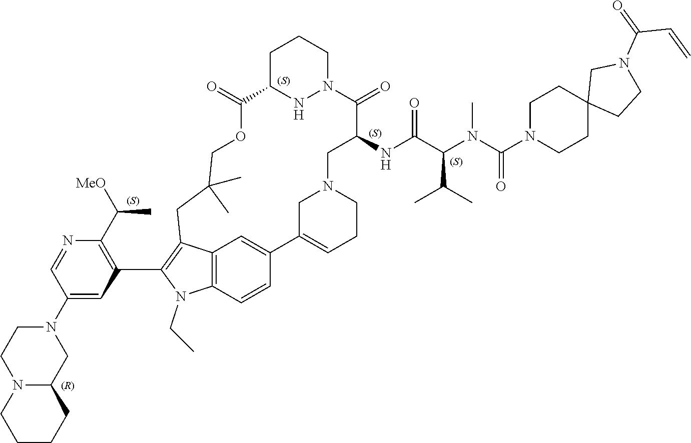 |
| EA84 | 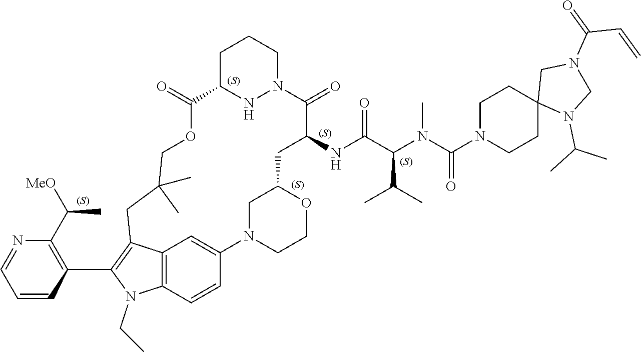 |
| EA85 | 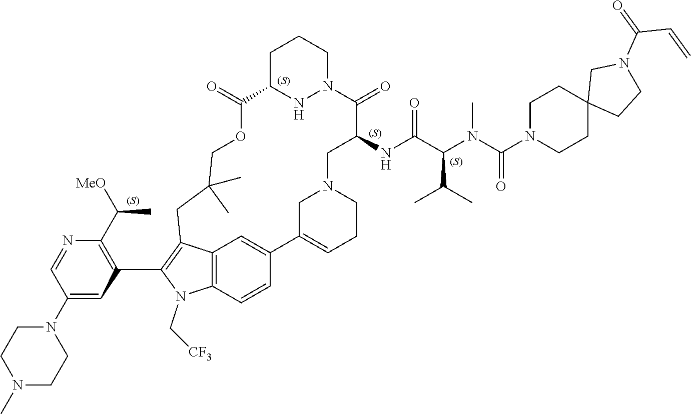 |
| EA86 | 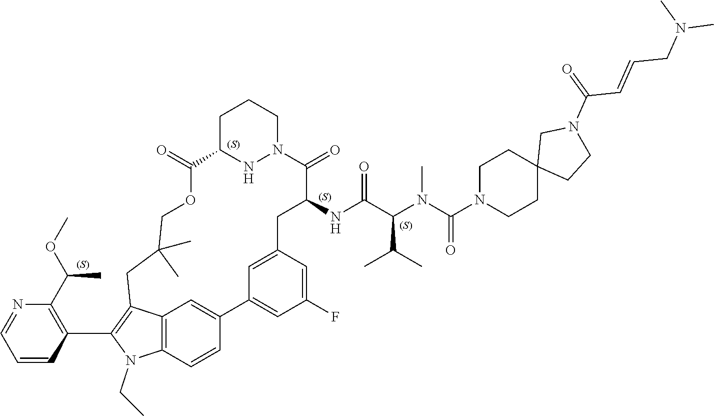 |
| EA87 | 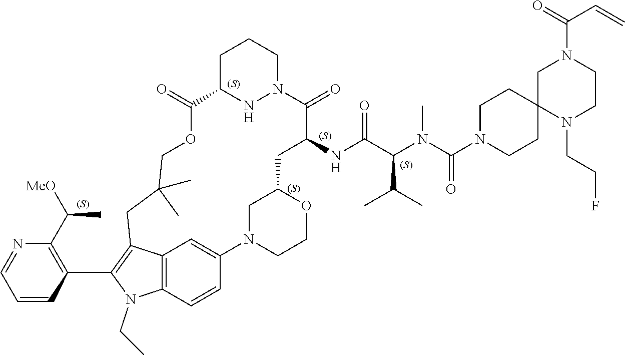 |
| EA88 | 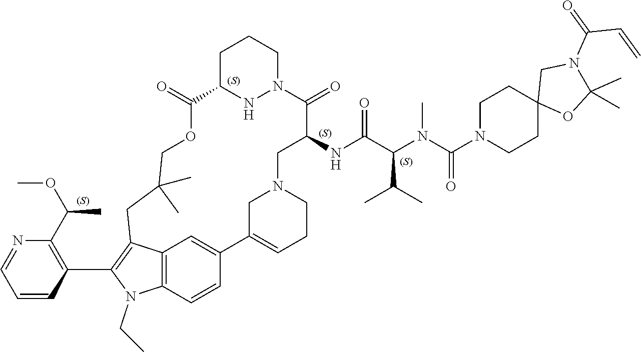 |
| EA89 | 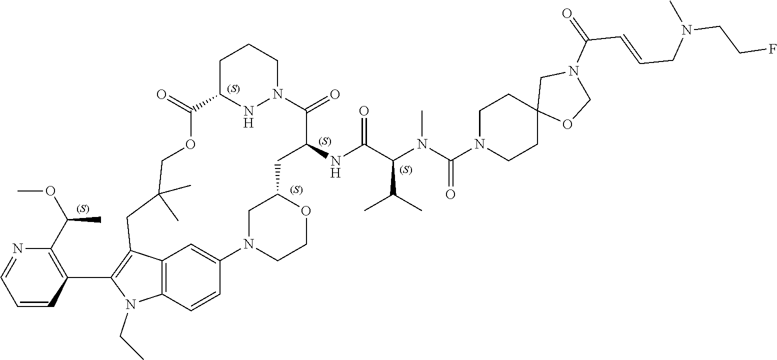 |
| EA90 | 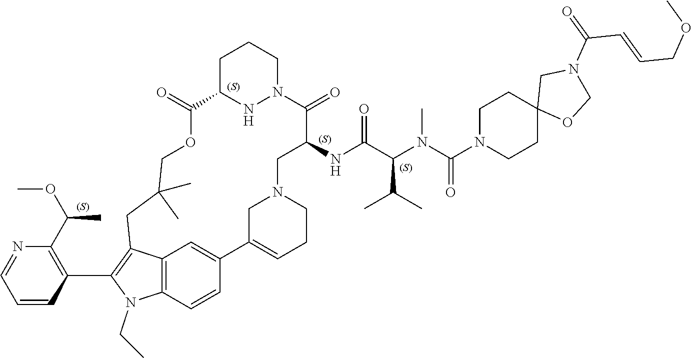 |
| EA91 | 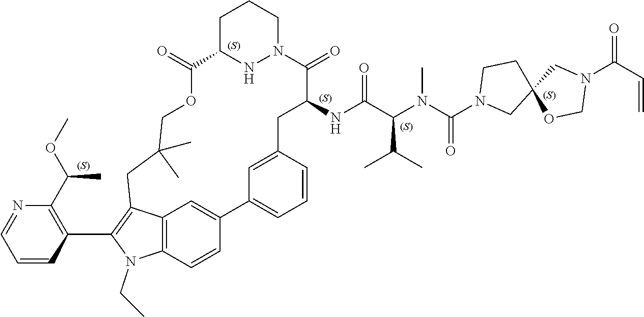 |
| EA92 | 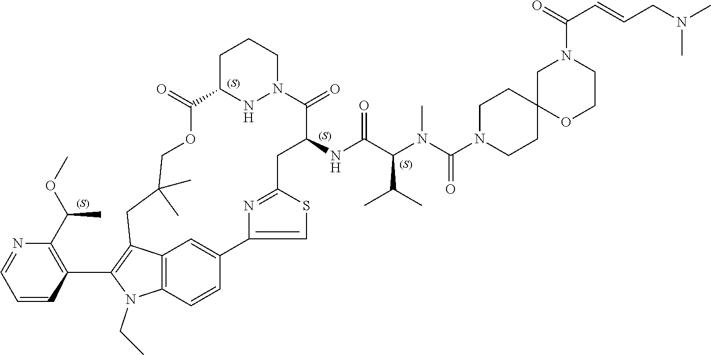 |
| EA93 | 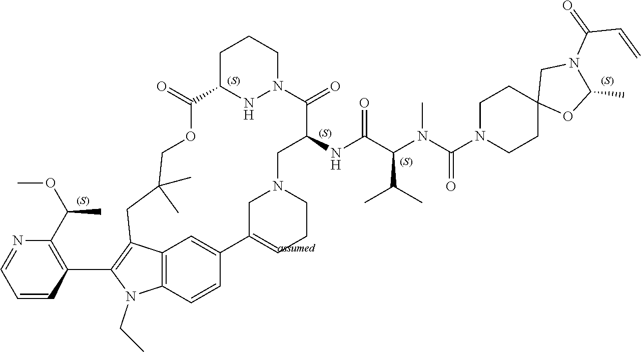 |
| EA94 | 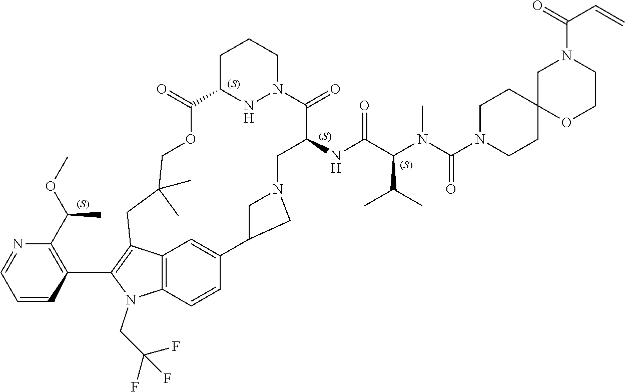 |
| EA95 | 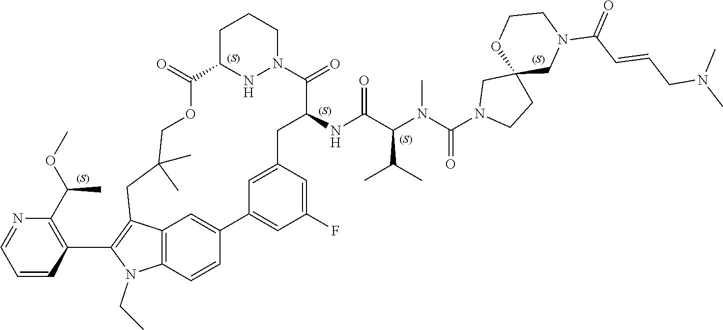 |
| EA96 | 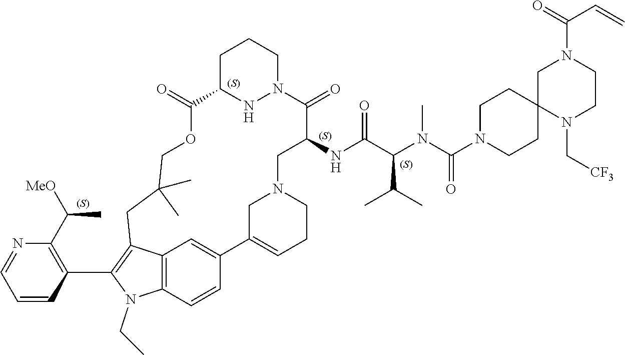 |
| EA97 | 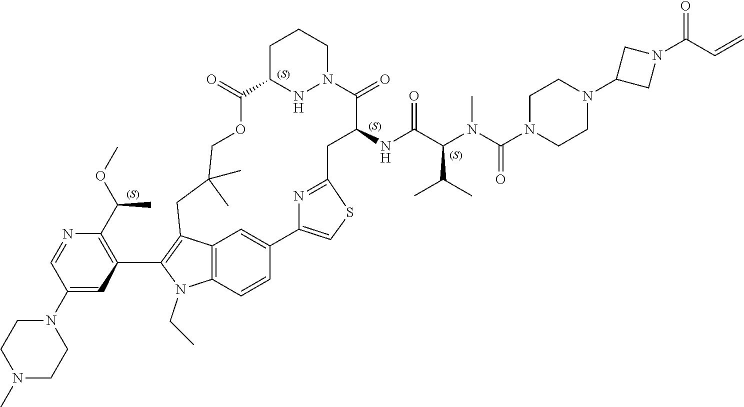 |
| EA98 | 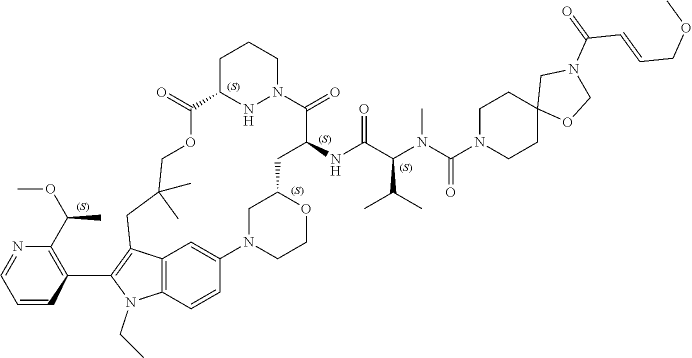 |
| EA99 | 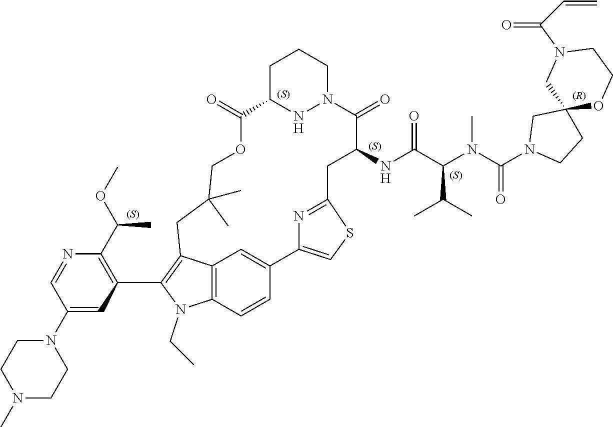 |
| EA100 | 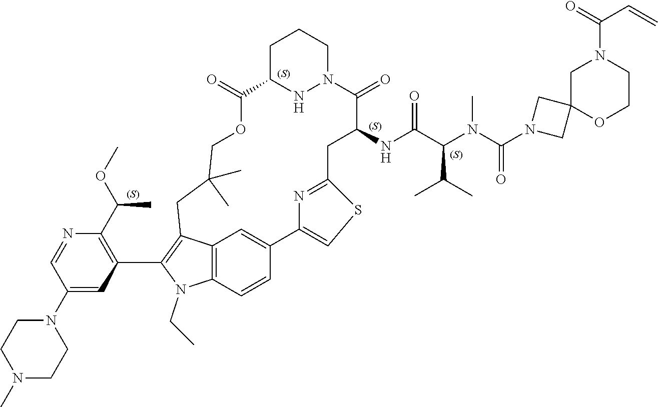 |
| EA101 | 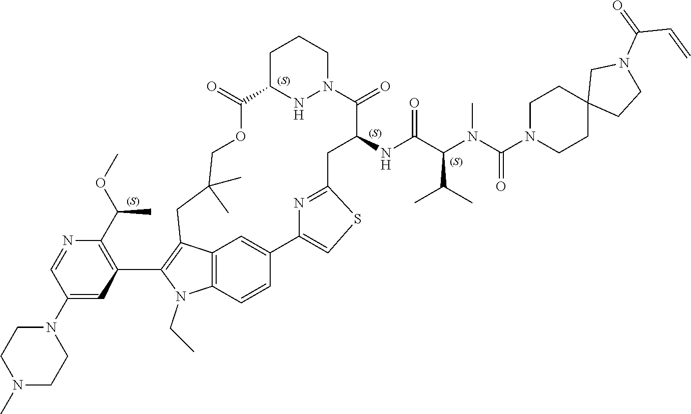 |
| EA102 | 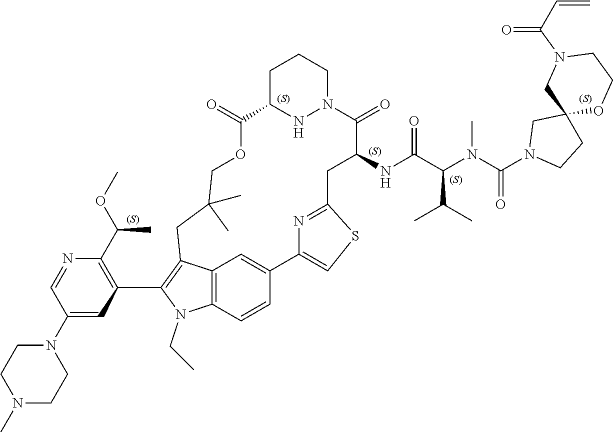 |
| EA103 | 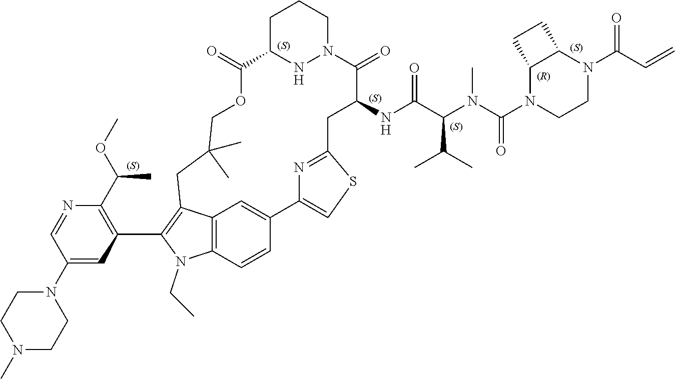 |
| EA104 | 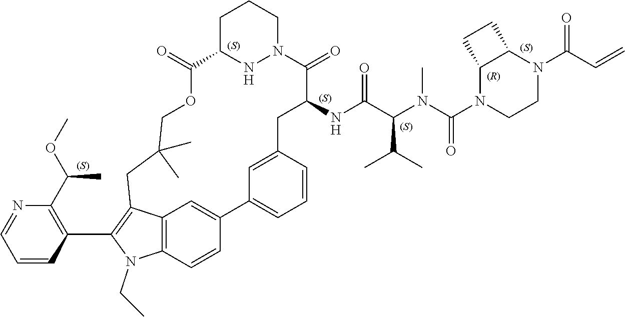 |
| EA105 | 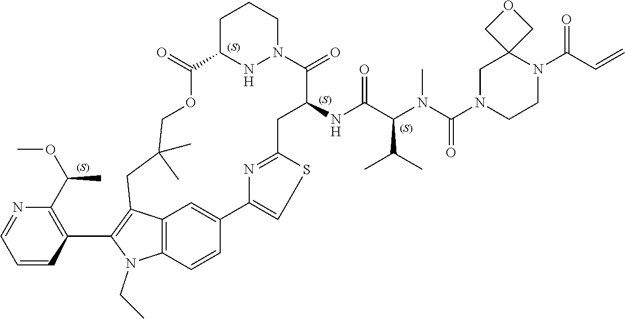 |
| EA105 | 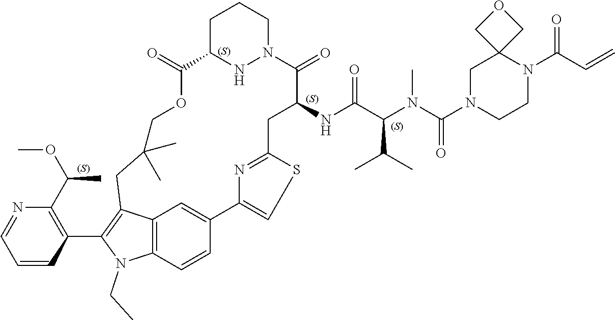 |
| EA106 | 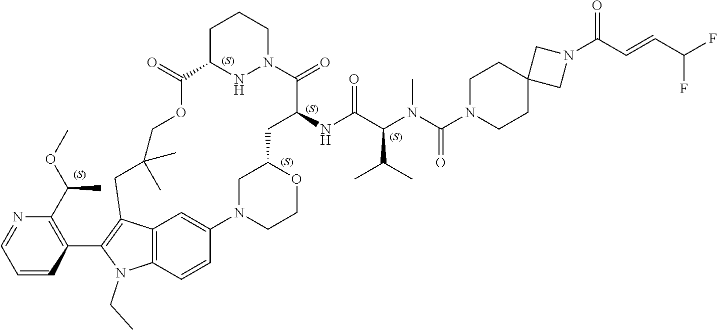 |
| EA107 | 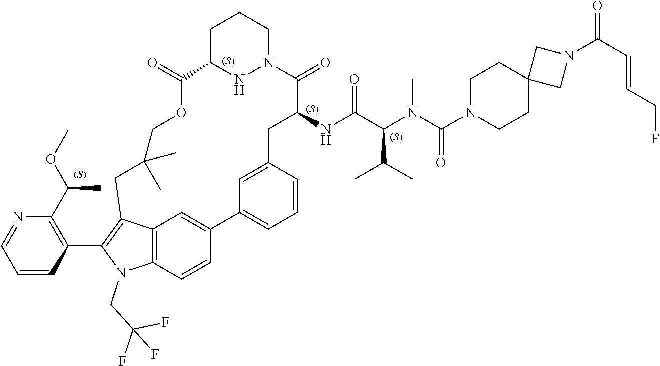 |
| EA108 | 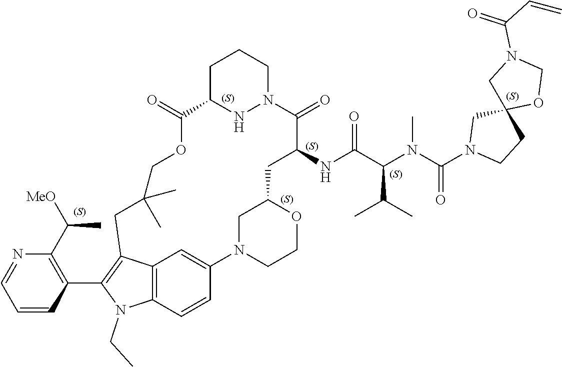 |
| EA109 | 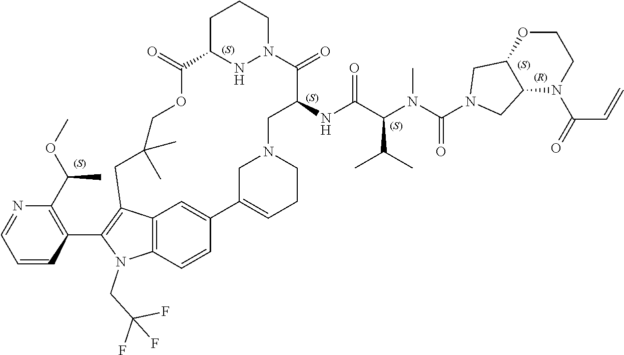 |
| EA110 | 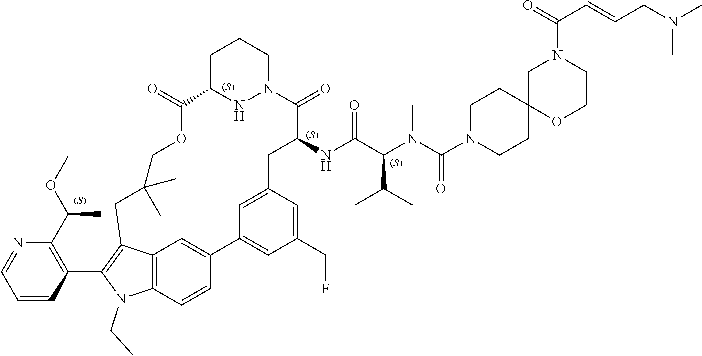 |
| EA111 | 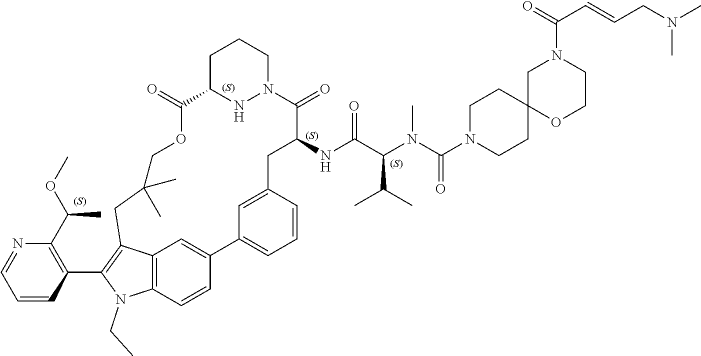 |
| EA112 | 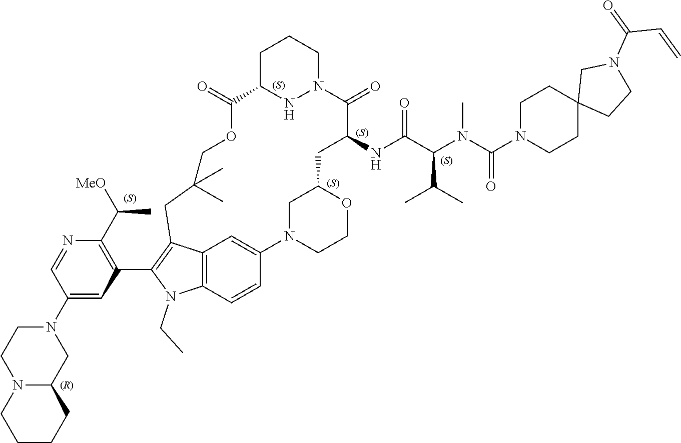 |
| EA113 | 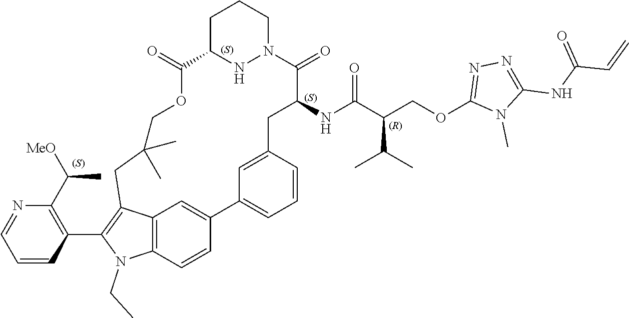 |
| EA114 | 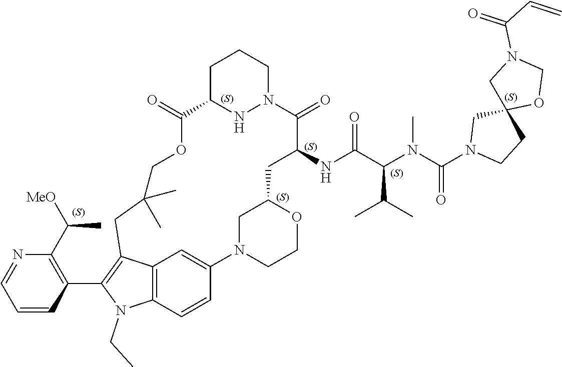 |
| EA115 | 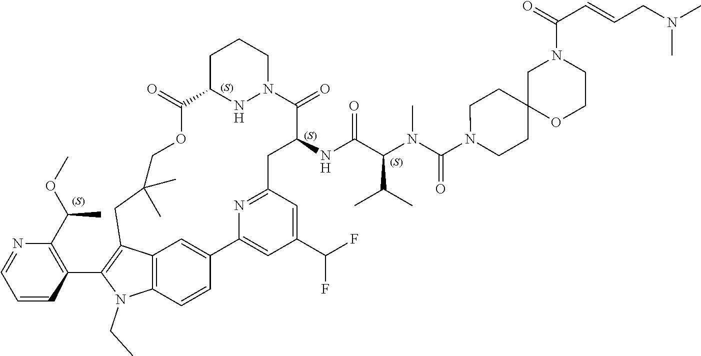 |
| EA116 | 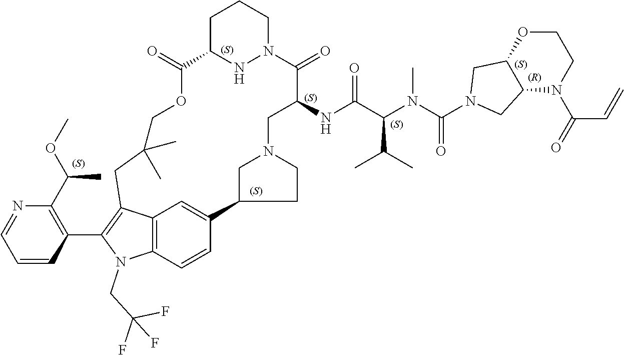 |
| EA117 | 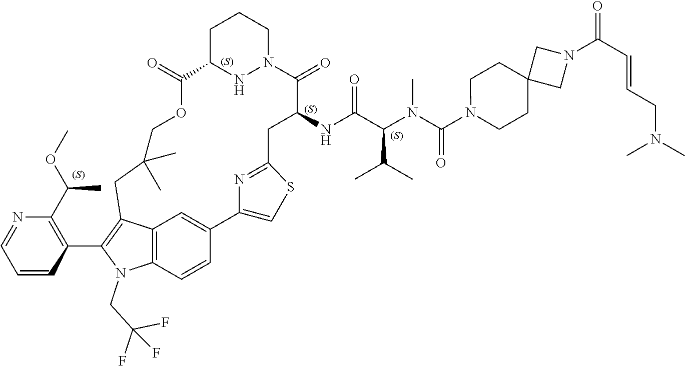 |
| EA118 | 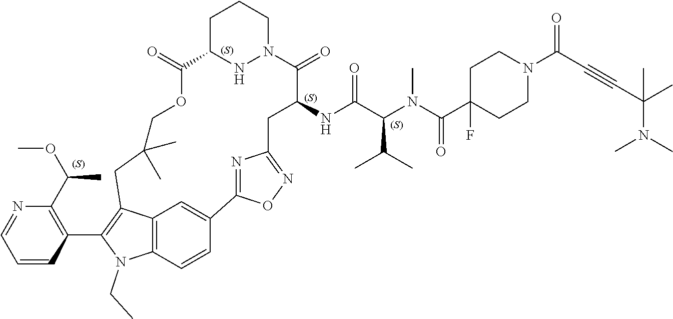 |
| EA119 | 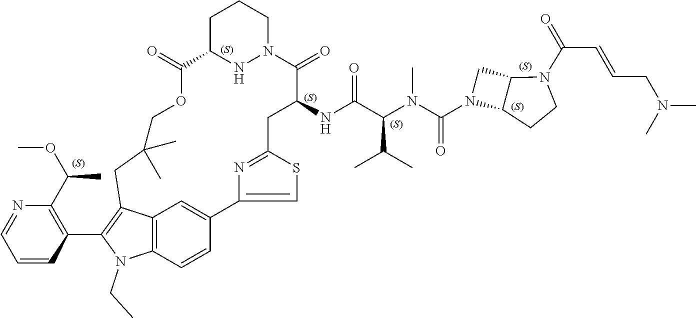 |
| EA120 | 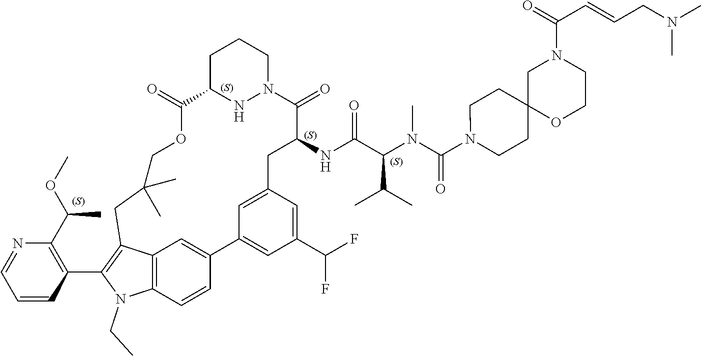 |
| EA121 | 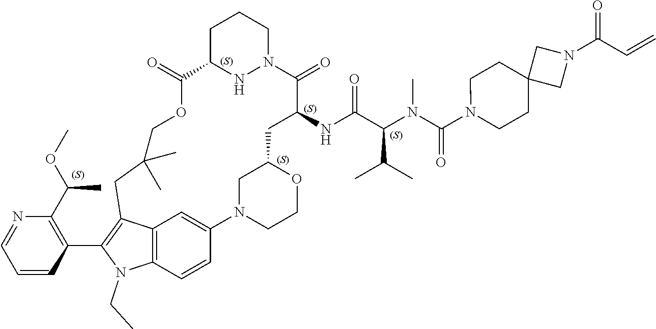 |
| EA122 | 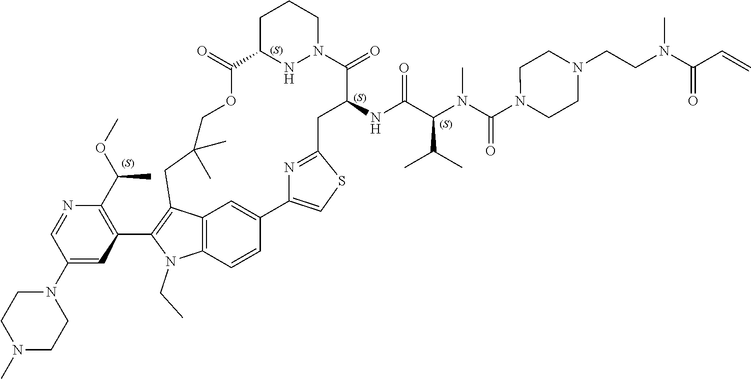 |
| EA123 | 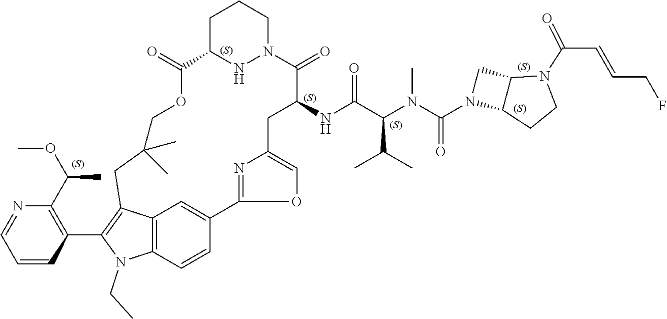 |
| EA124 | 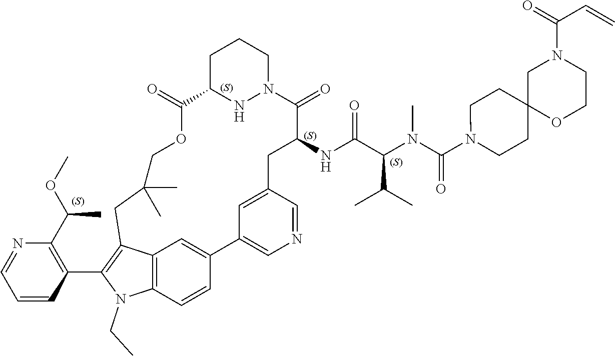 |
| EA125 | 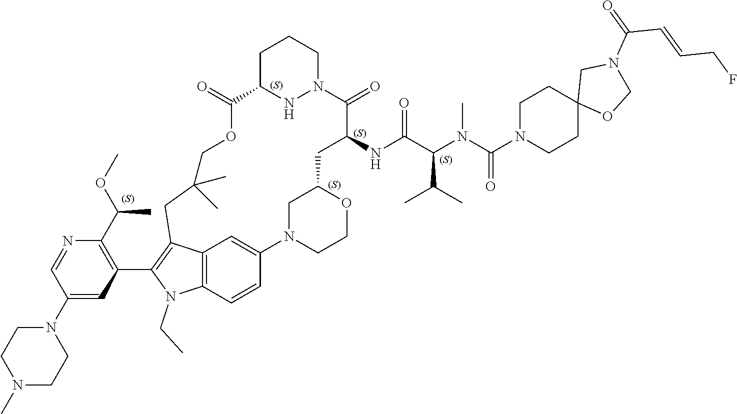 |
| EA126 | 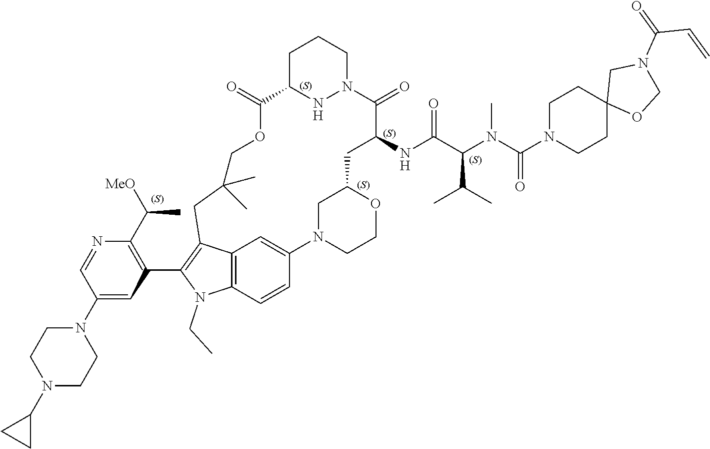 |
| EA127 | 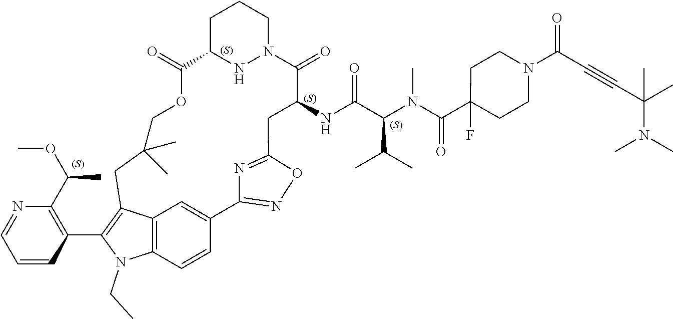 |
| EA128 | 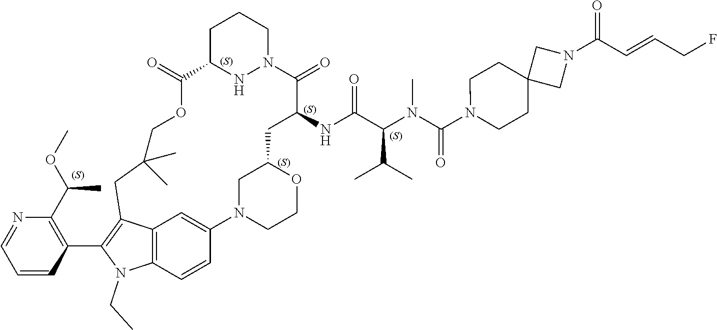 |
| EA129 | 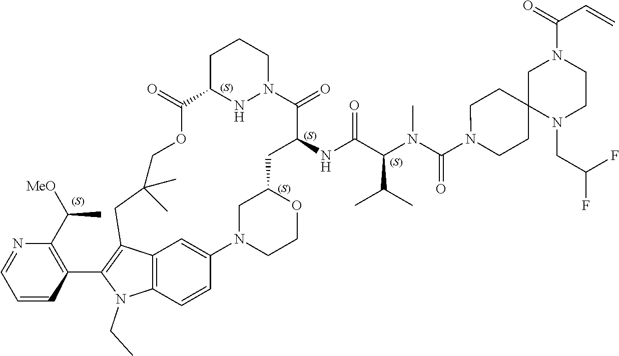 |
| EA130 | 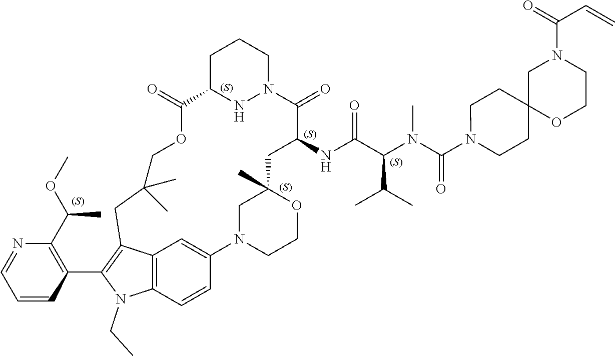 |
| EA131 | 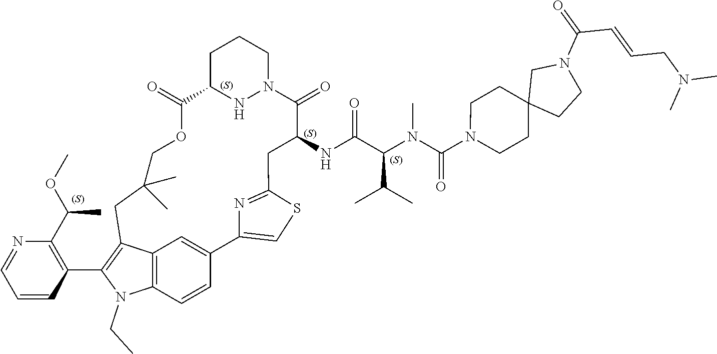 |
| EA132 | 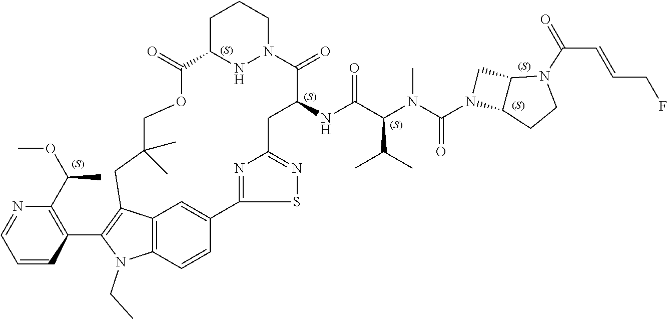 |
| EA133 | 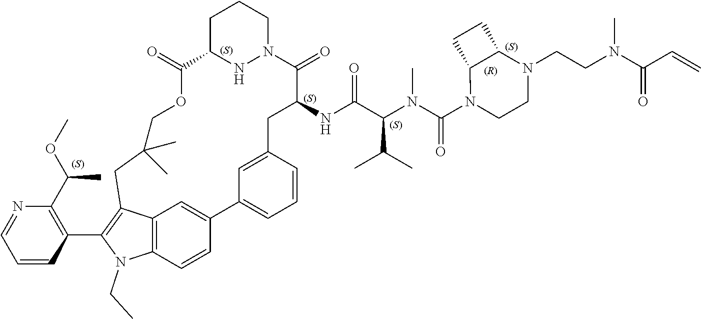 |
| EA134 | 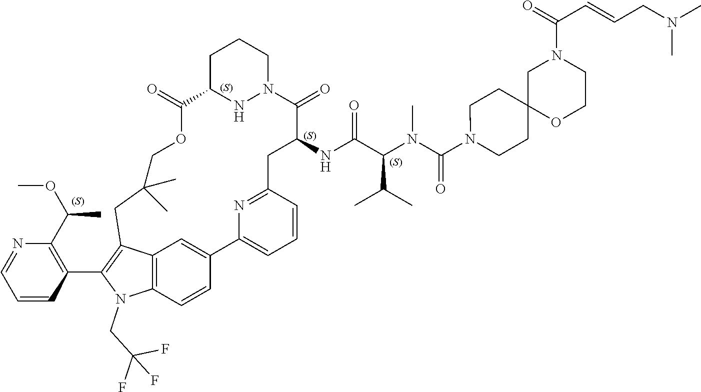 |
| EA135 | 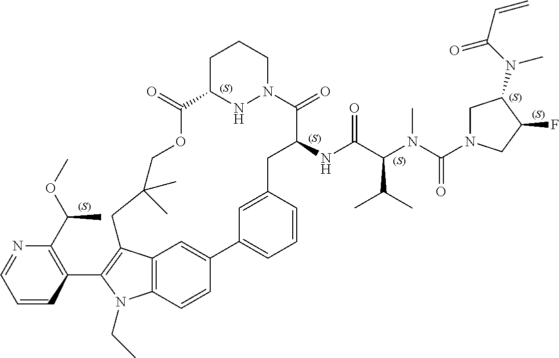 |
| EA136 | 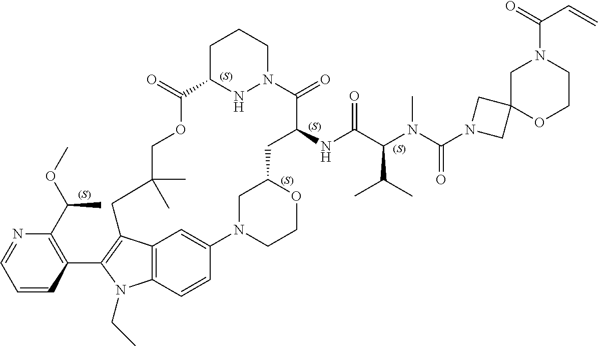 |
| EA137 | 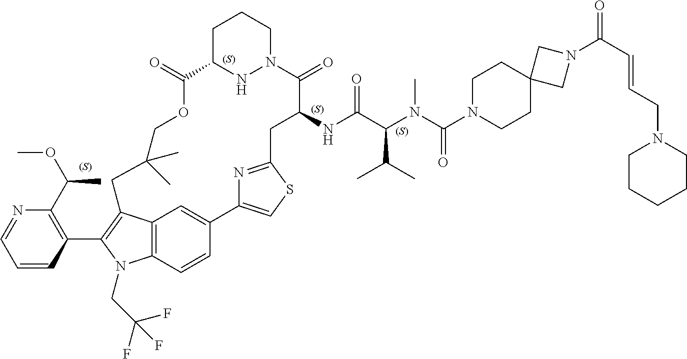 |
| EA138 | 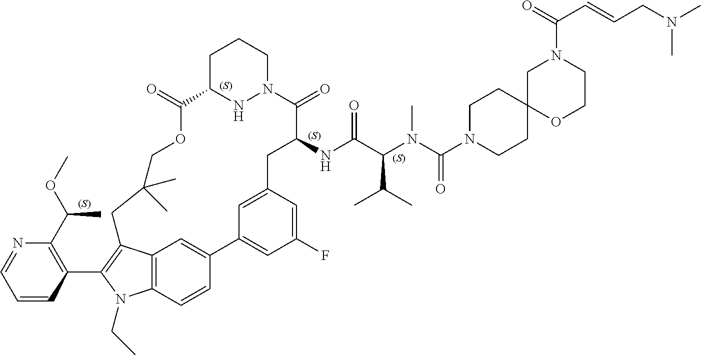 |
| EA139 | 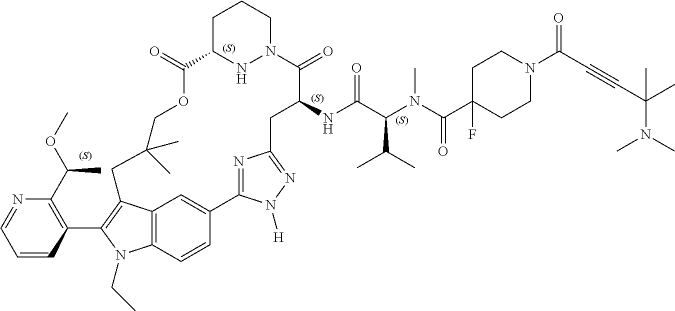 |
| EA140 | 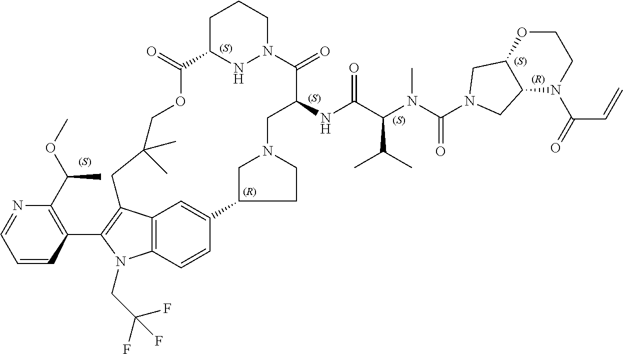 |
| EA141 | 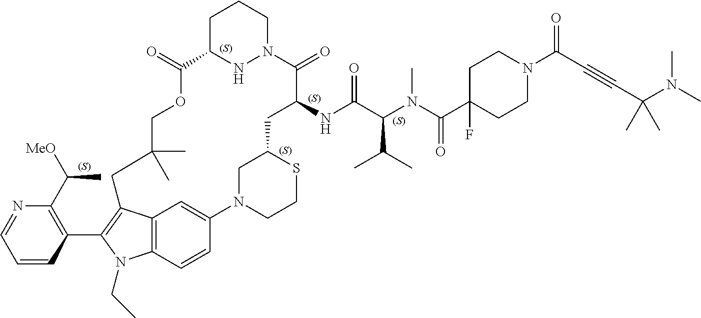 |
| EA142 | 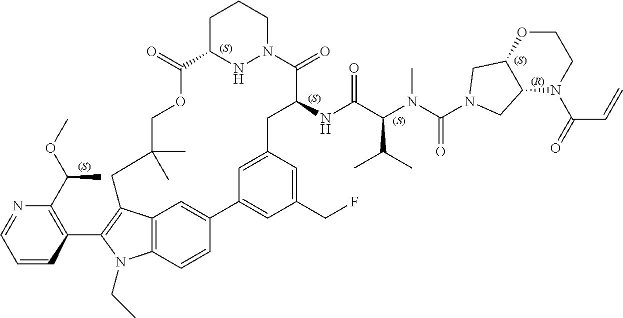 |
| EA143 | 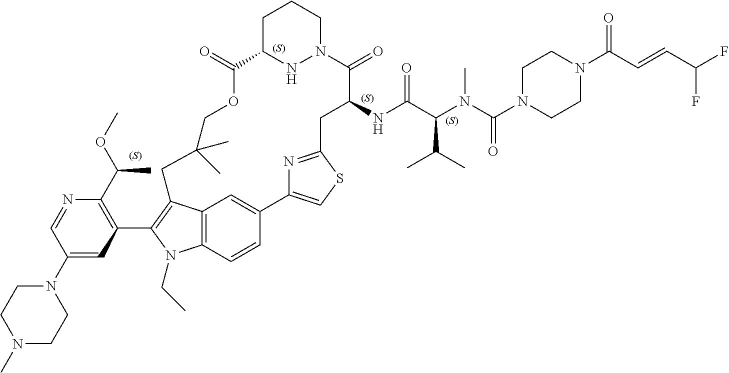 |
| EA144 | 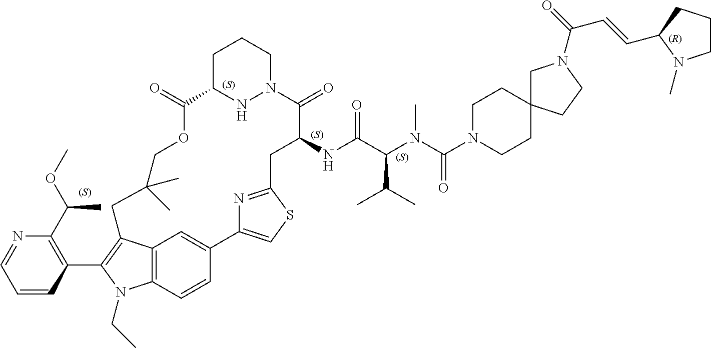 |
| EA145 | 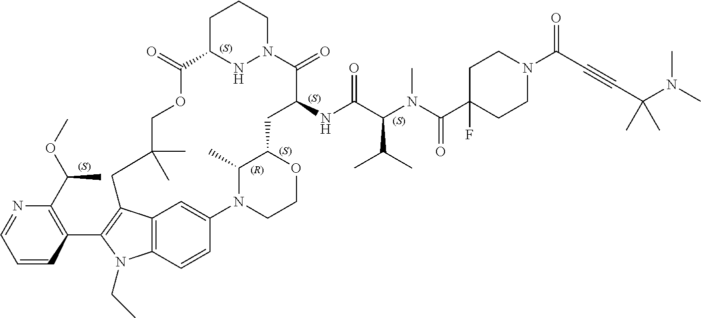 |
| EA146 | 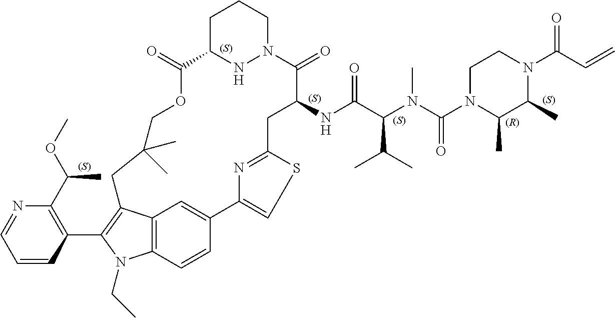 |
| EA147 | 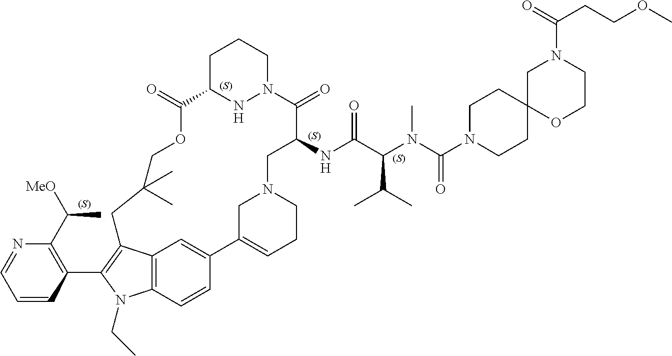 |
| EA148 | 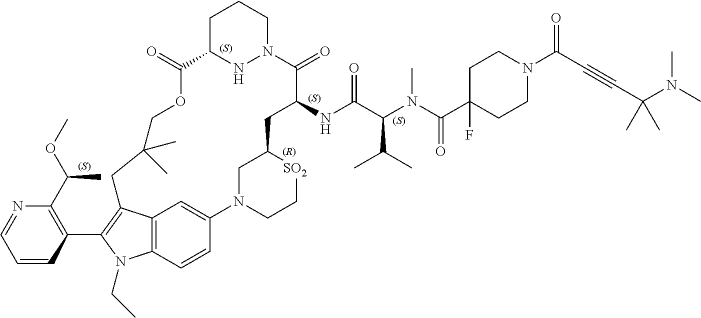 |
| EA149 | 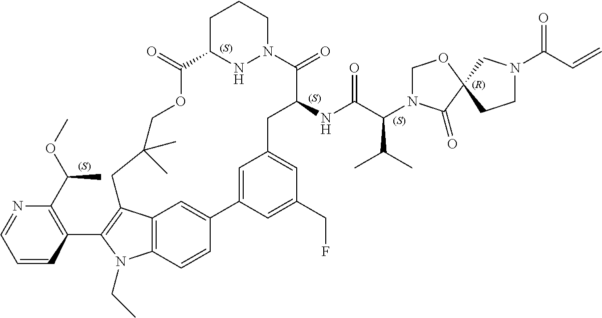 |
| EA150 | 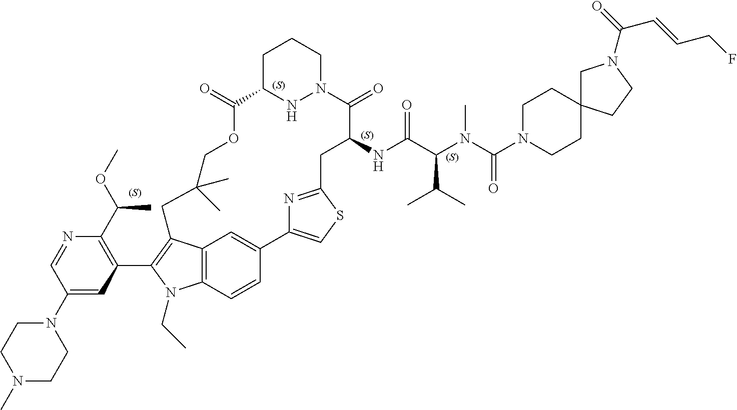 |
| EA151 | 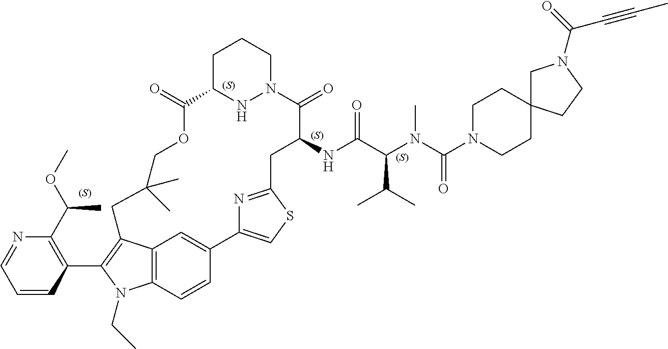 |
| EA152 | 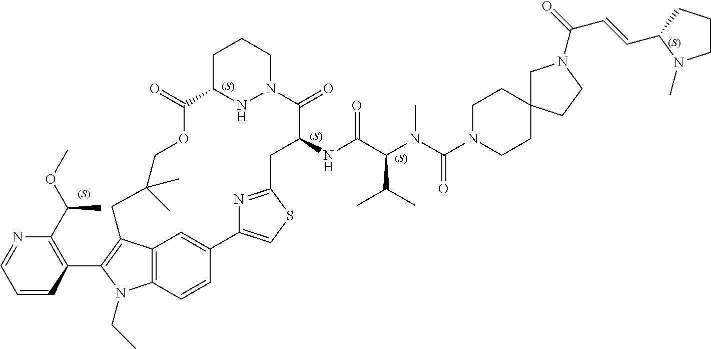 |
| EA153 | 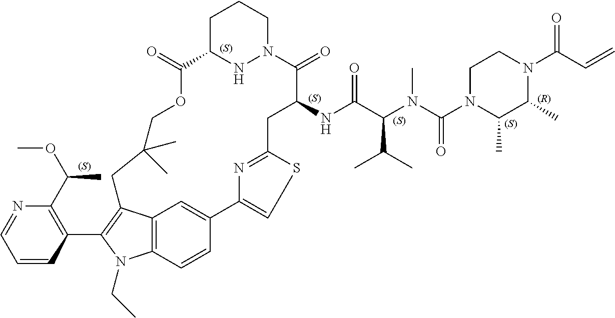 |
| EA154 | 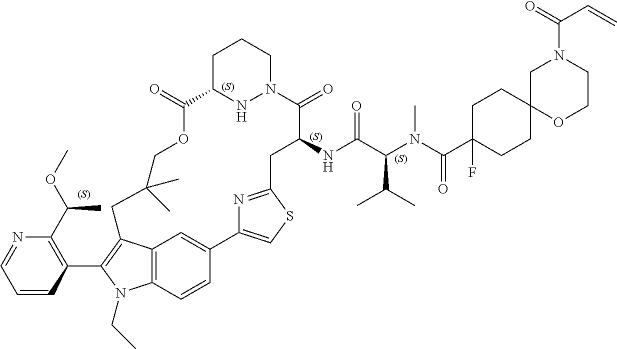 |
| EA155 | 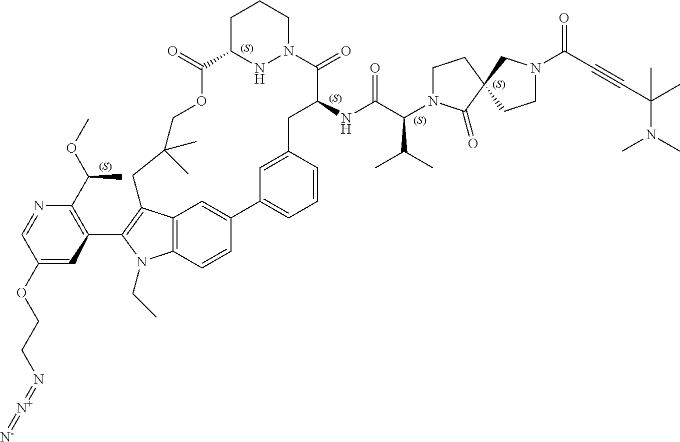 |
| EA156 | 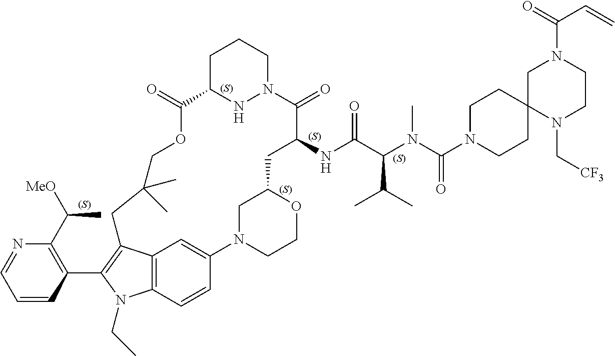 |
| EA157 | 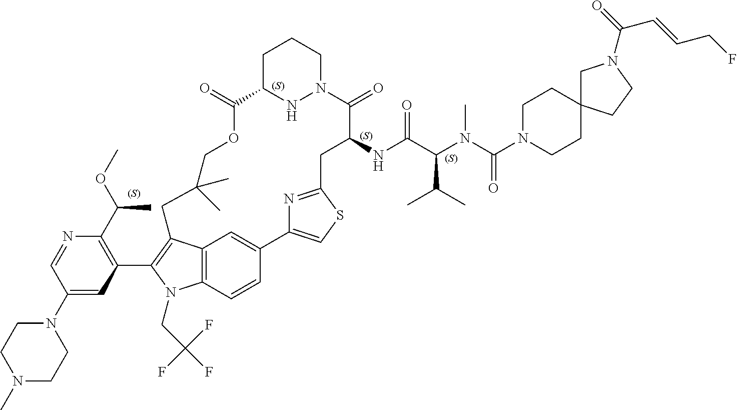 |
| EA158 | 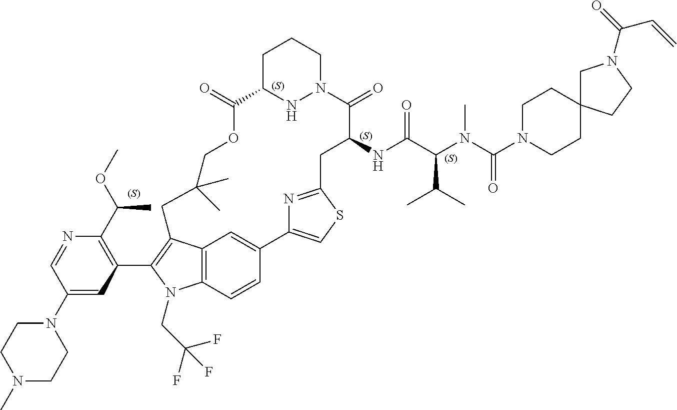 |
| EA159 | 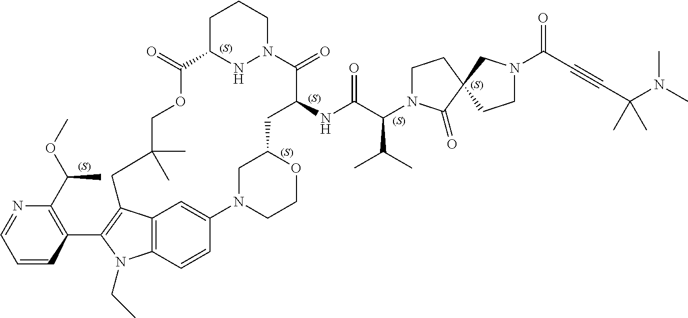 |
| EA160 | 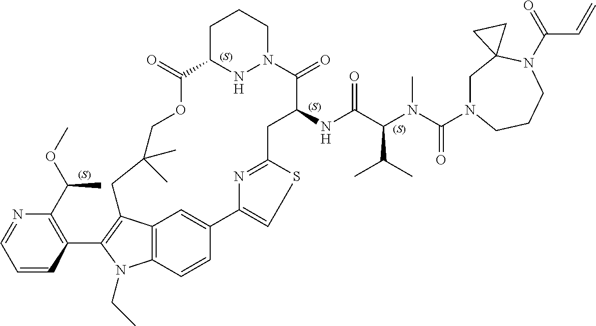 |
| EA161 | 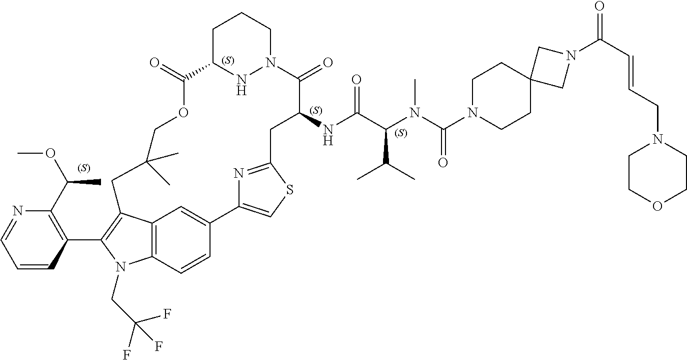 |
| EA162 | 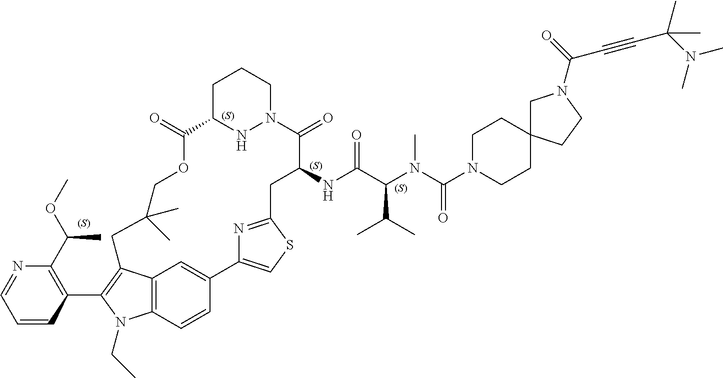 |
| EA163 | 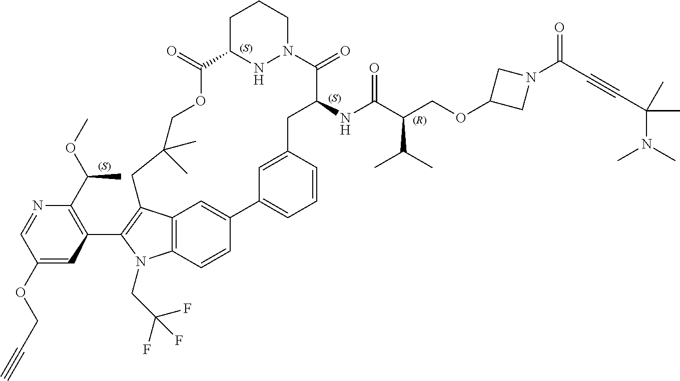 |
| EA164 | 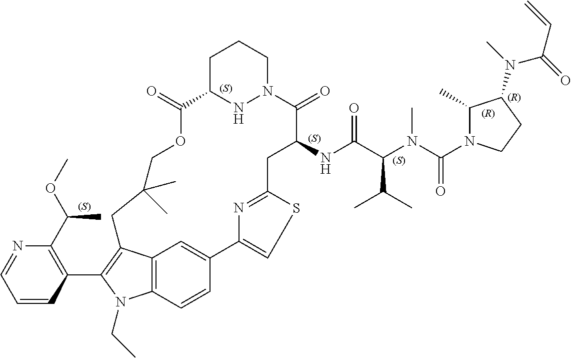 |
| EA165 | 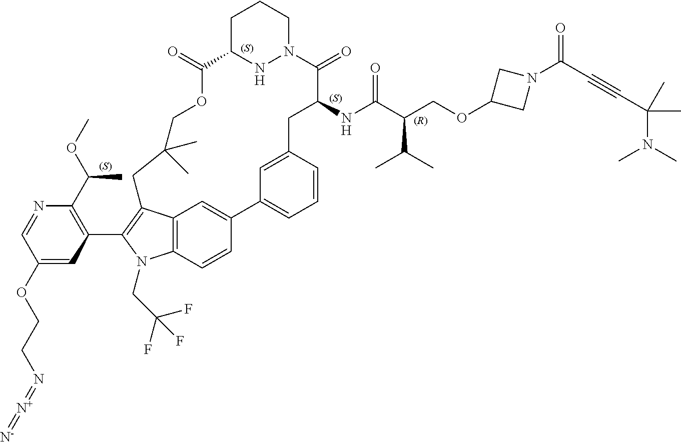 |
| EA166 | 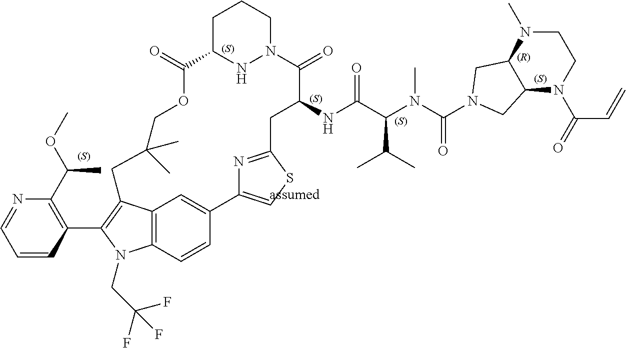 |
| EA167 | 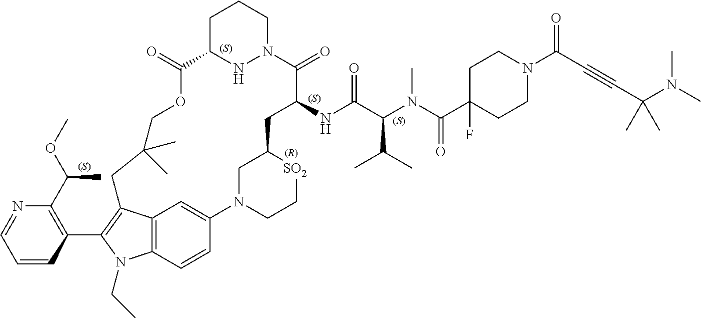 |
| EA168 | 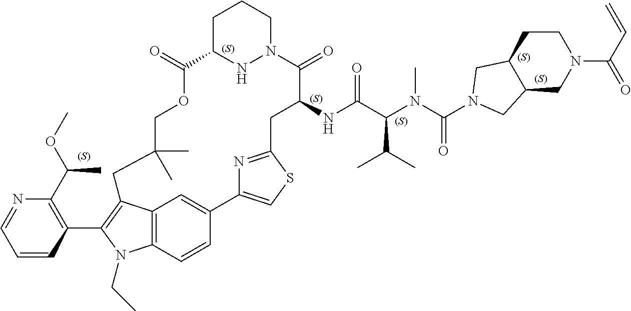 |
| EA169 | 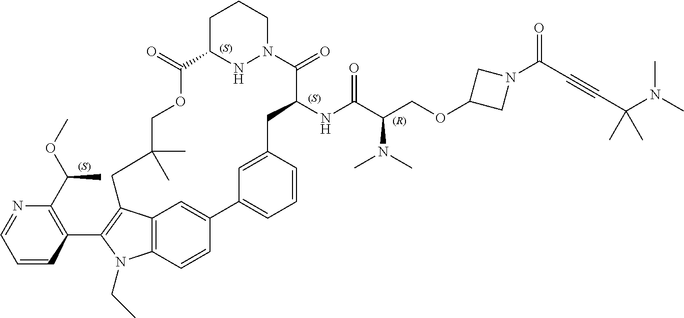 |
| EA170 | 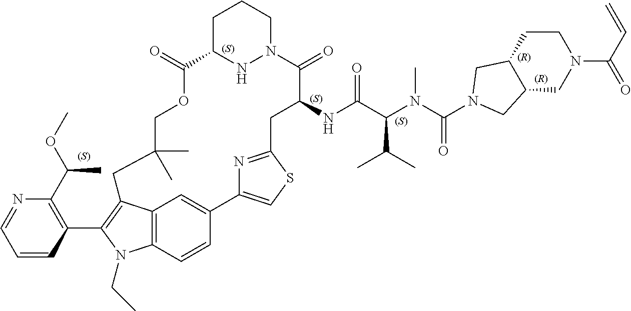 |
| EA171 | 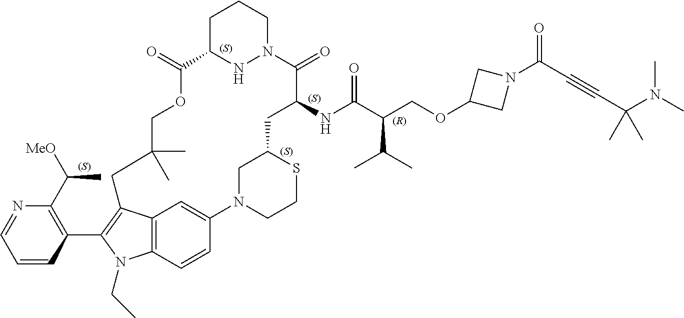 |
| EA172 | 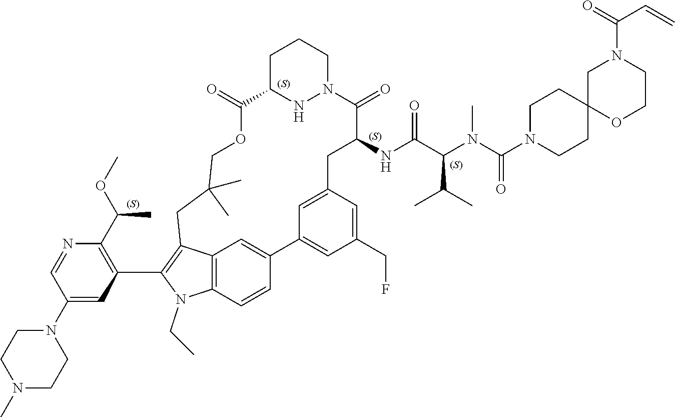 |
| EA173 | 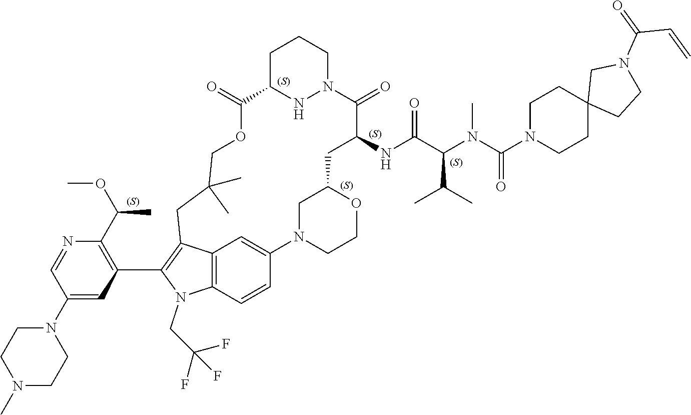 |
| EA174 | 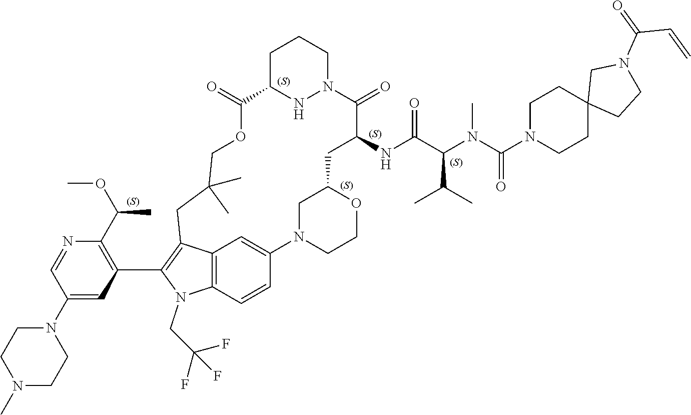 |
| EA175 | 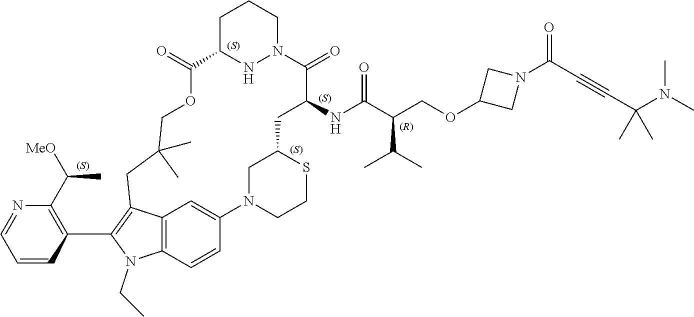 |
| EA176 | 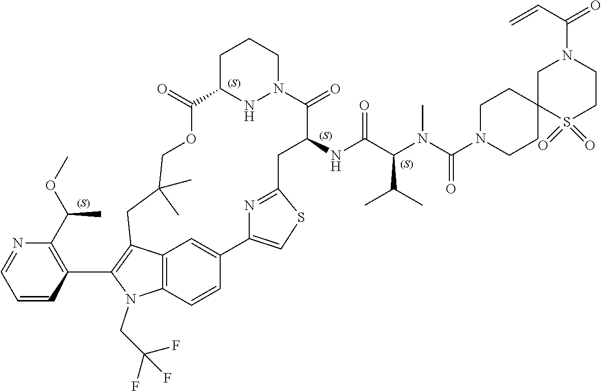 |
| EA177 | 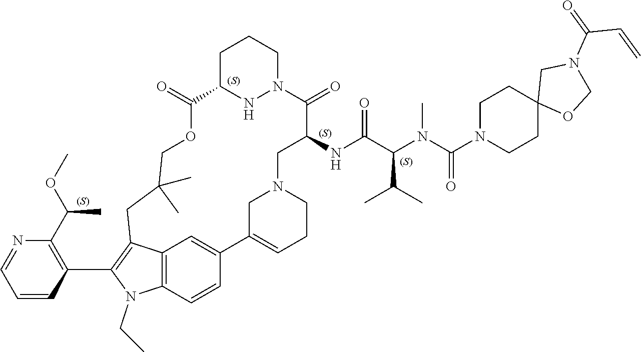 |
| EA178 | 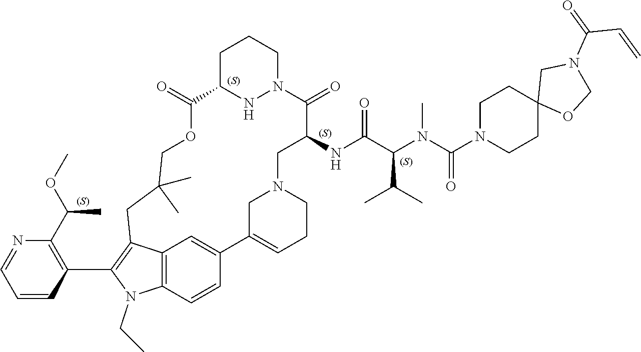 |
| EA179 | 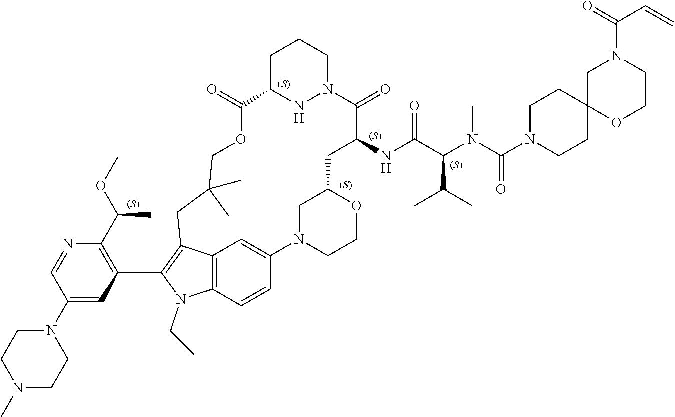 |
| EA180 | 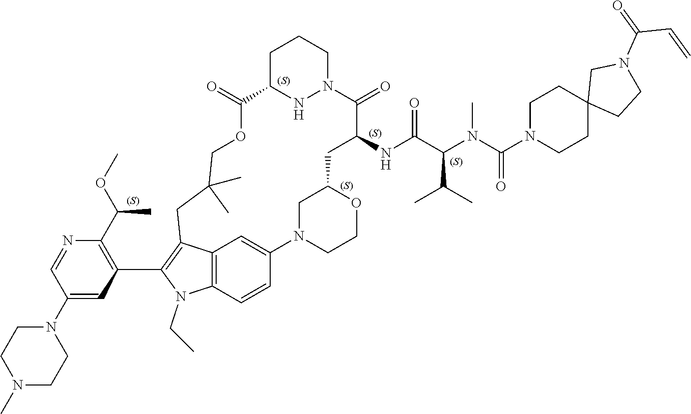 |
| EA181 | 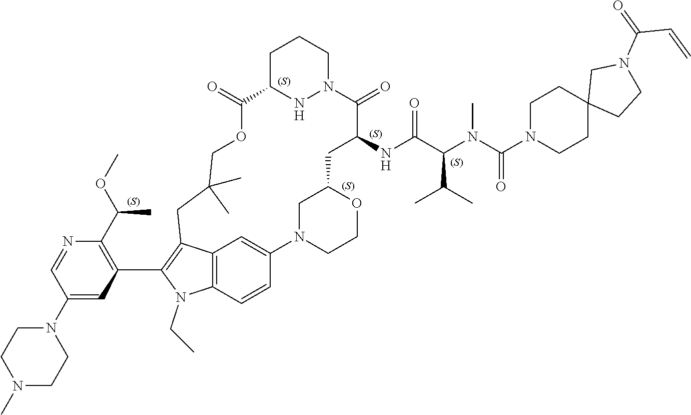 |
| EA182 | 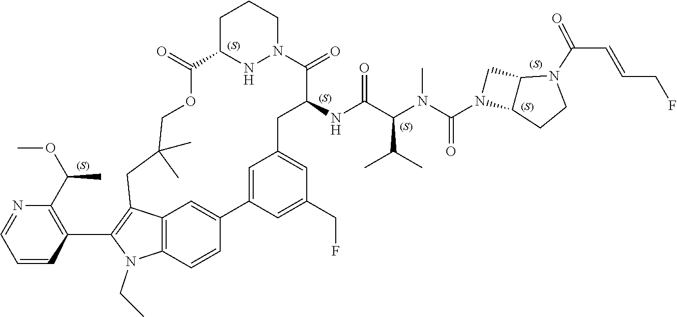 |
| EA183 | 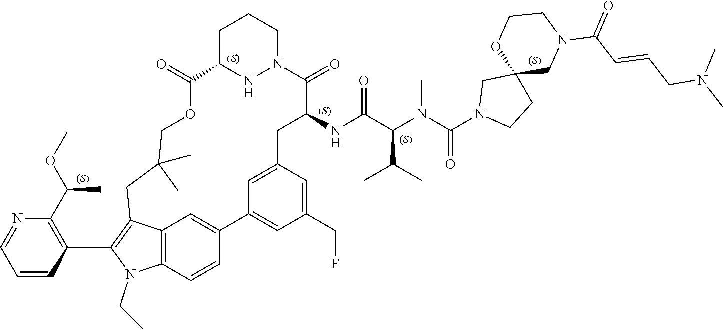 |
| EA184 | 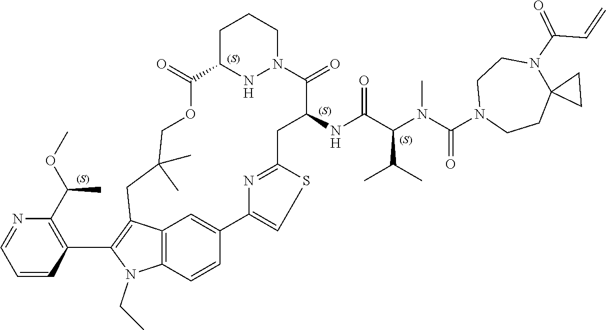 |
| EA185 | 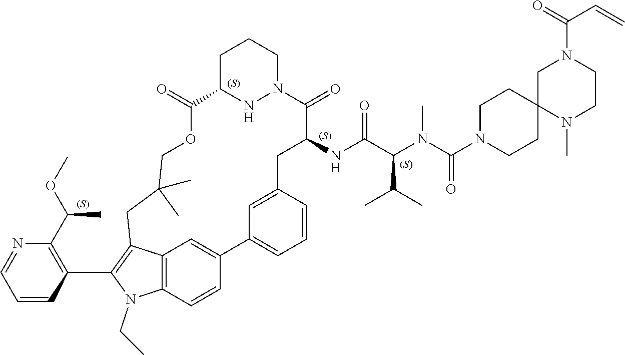 |
| EA186 | 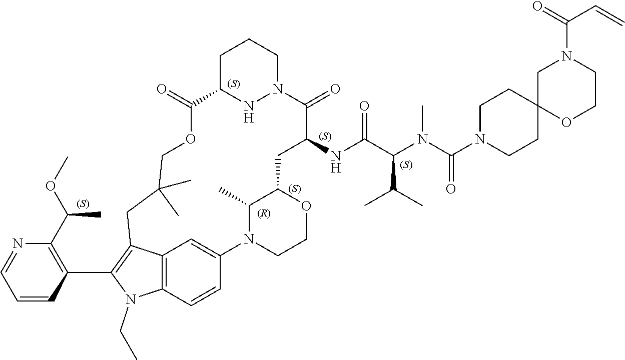 |
| EA187 | 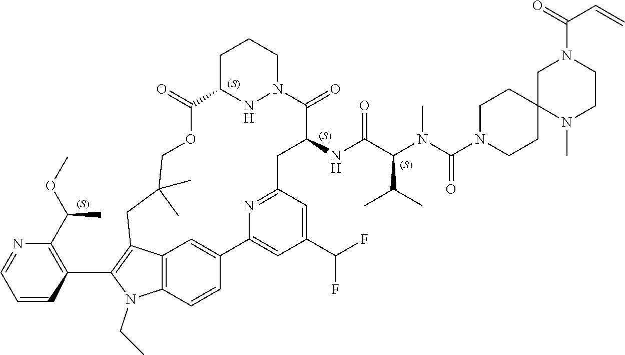 |
| EA188 | 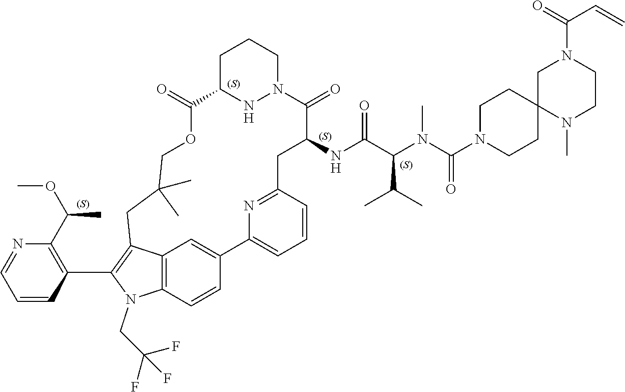 |
| EA189 | 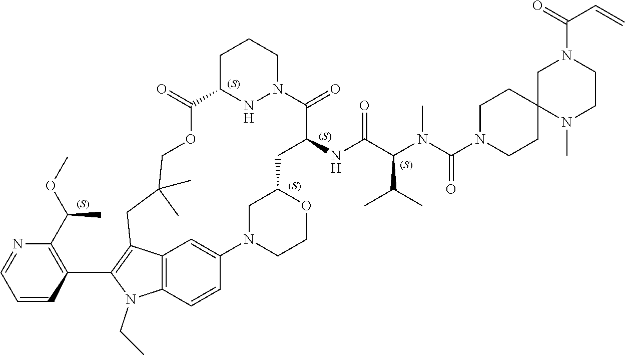 |
| EA190 | 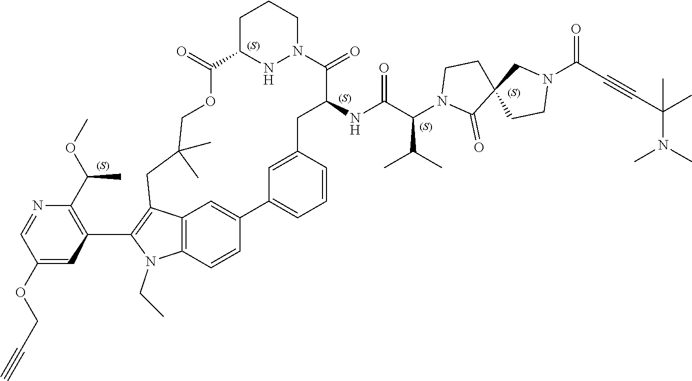 |
| EA191 | 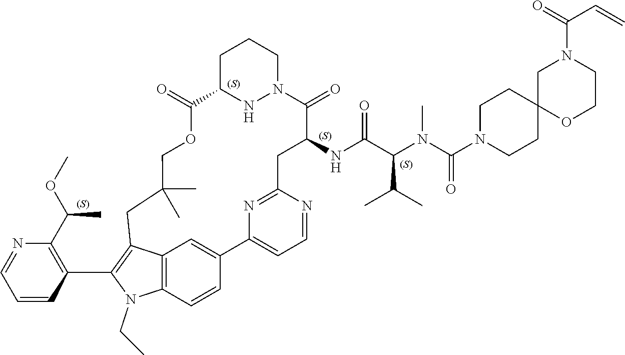 |
| EA192 | 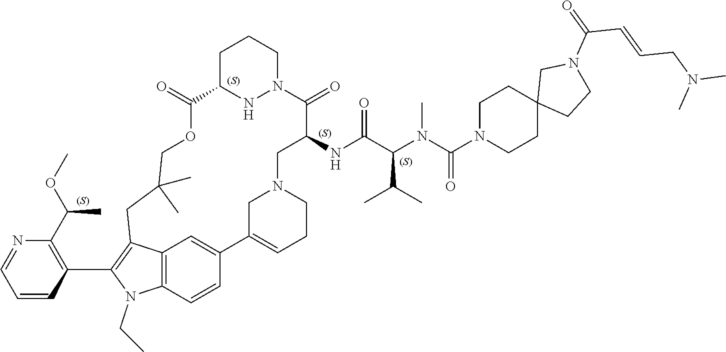 |
| EA193 | 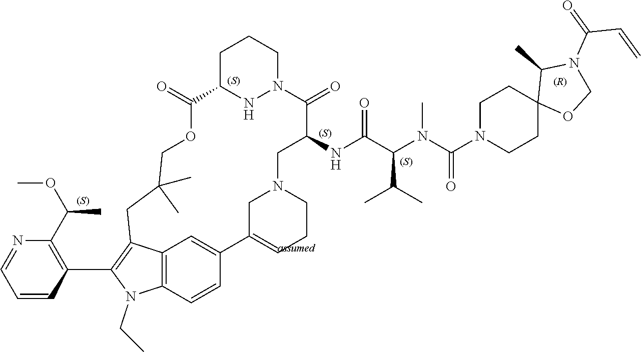 |
| EA194 | 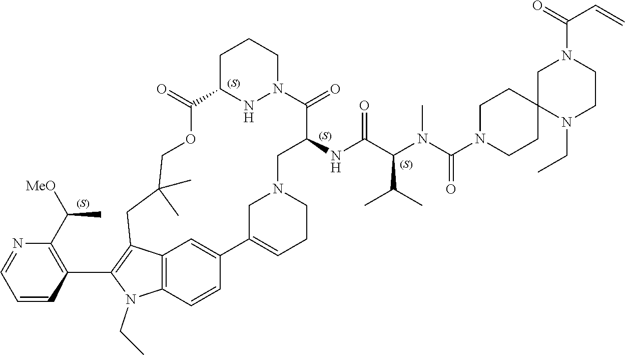 |
| EA195 | 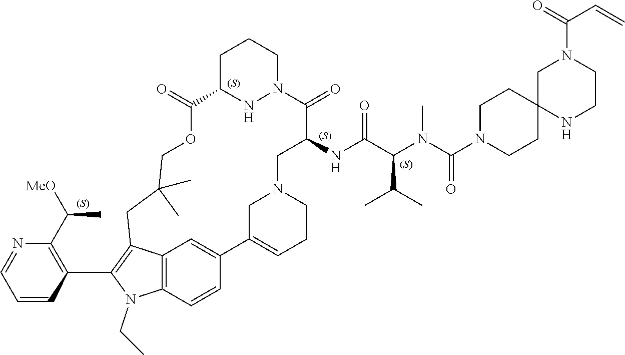 |
| EA196 | 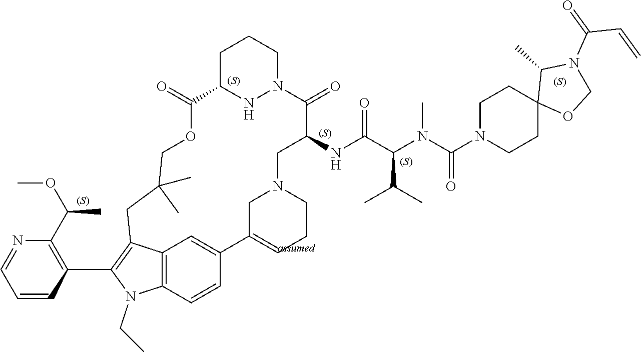 |
| EA197 | 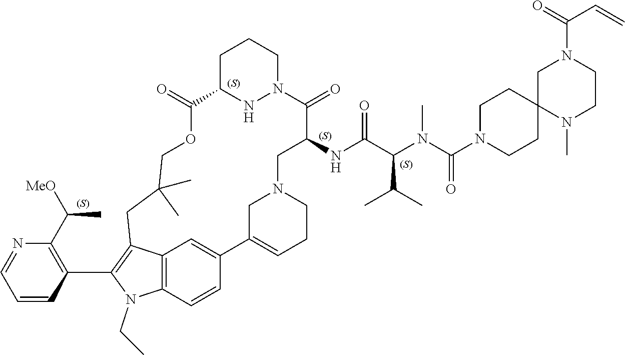 |
| EA198 | 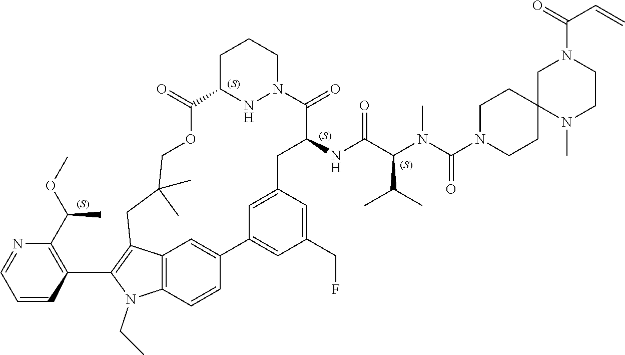 |
| EA199 | 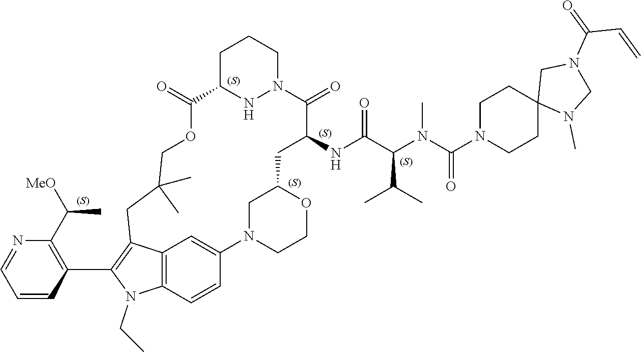 |
| EA200 | 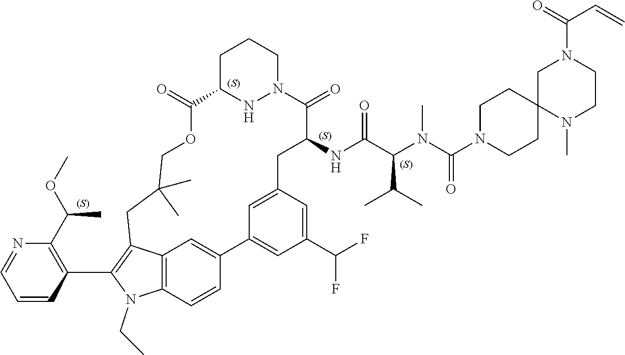 |
| EA201 | 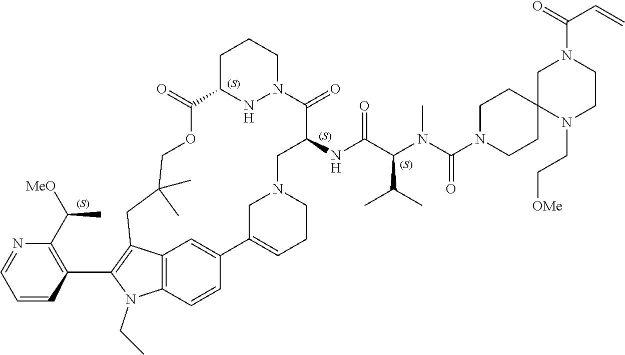 |
| EA202 | 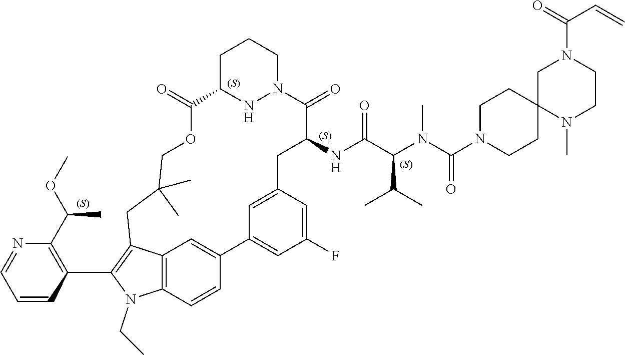 |
| EA203 | 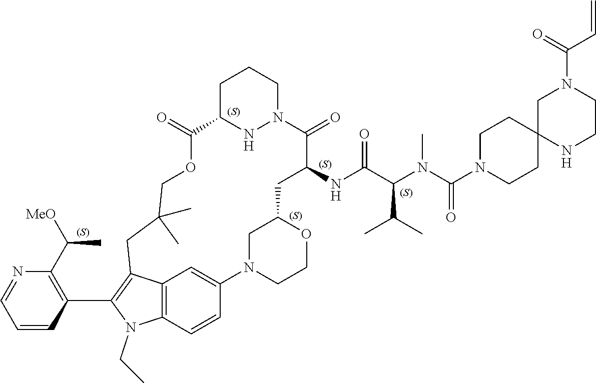 |
| EA204 | 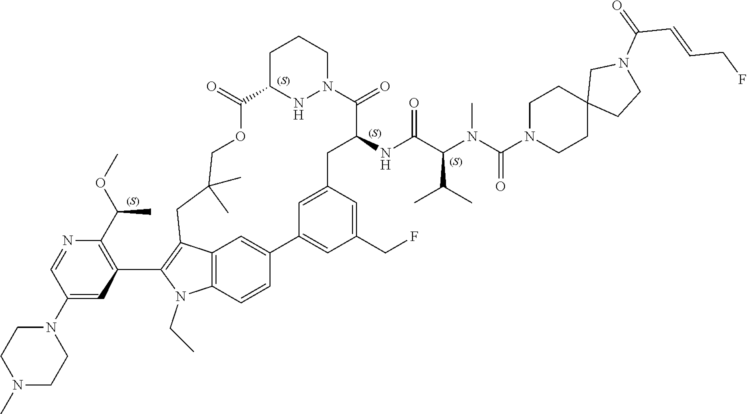 |
| EA205 | 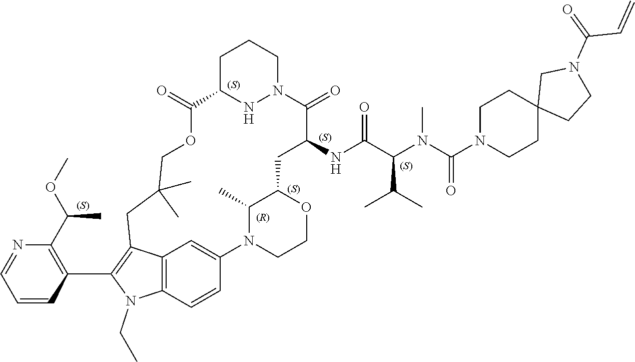 |
| EA206 | 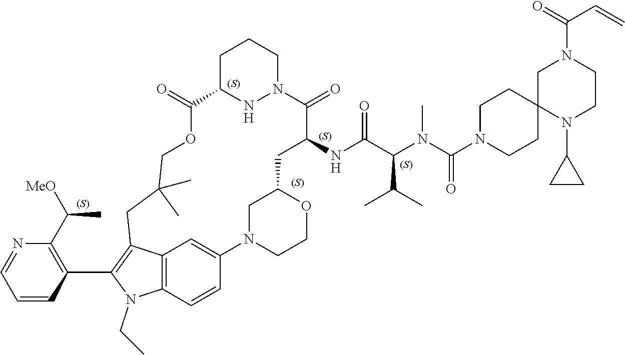 |
| EA207 | 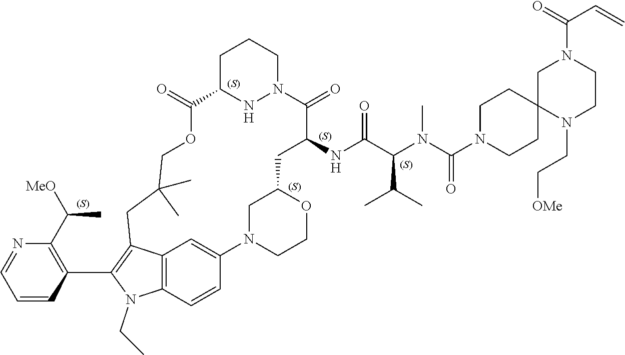 |
| EA208 | 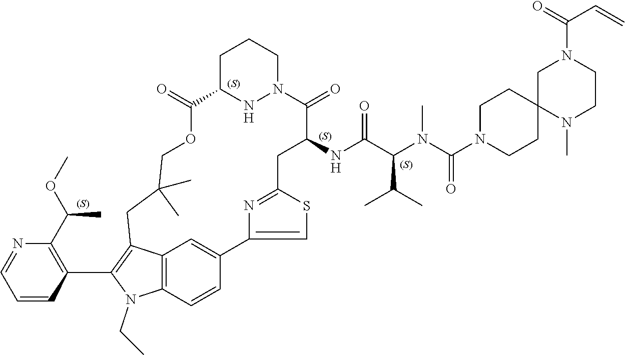 |
| EA209 | 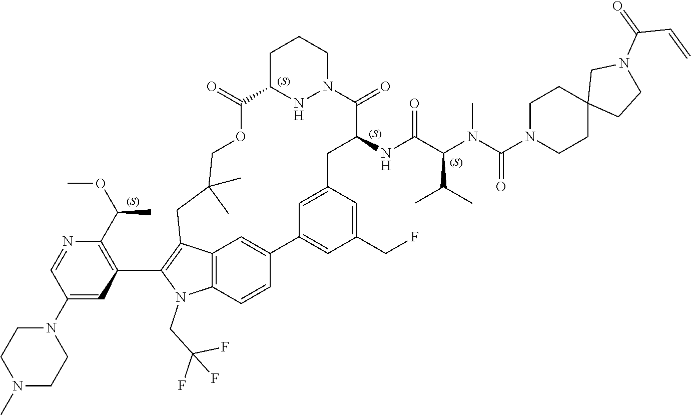 |

Also provided is a pharmaceutical composition comprising a compound of the present invention, or a pharmaceutically acceptable salt thereof, and a pharmaceutically acceptable excipient.

In some embodiments, the RAS(ON) inhibitor is provided as a conjugate, or salt thereof, comprising the structure of Formula EV:

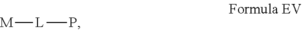

wherein L is a linker;
P is a monovalent organic moiety; and
M has the structure of Formula EVIa:

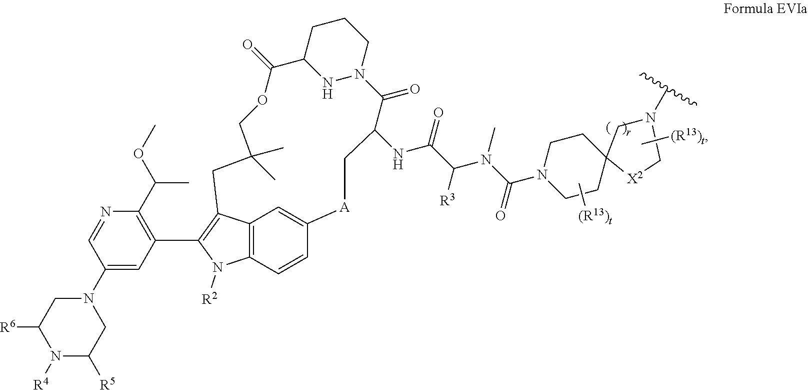

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl;
X² is O, C(R¹¹)₂, NR¹², S, or SO₂;
r is 1 or 2;
each t is, independently, 0, 1, or 2;
R¹¹ and R¹² are each, independently, hydrogen, optionally substituted C₁-C₄ alkyl, optionally substituted C₂-C₄ heteroalkyl, or optionally substituted 3 to 5-membered cycloalkyl;
each R¹³ is, independently, —CH₃; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or R⁴ and R⁶ combine with the atoms to which they are attached to form an optionally substituted 3 to 8-membered cycloalkyl or an optionally substituted 3 to 8-membered heterocycloalkyl.

In some embodiments, the RAS(ON) inhibitor is provided as a conjugate, or salt thereof, comprising the structure of Formula EV:

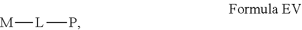

wherein L is a linker;
P is a monovalent organic moiety; and
M has the structure of Formula EVIb:

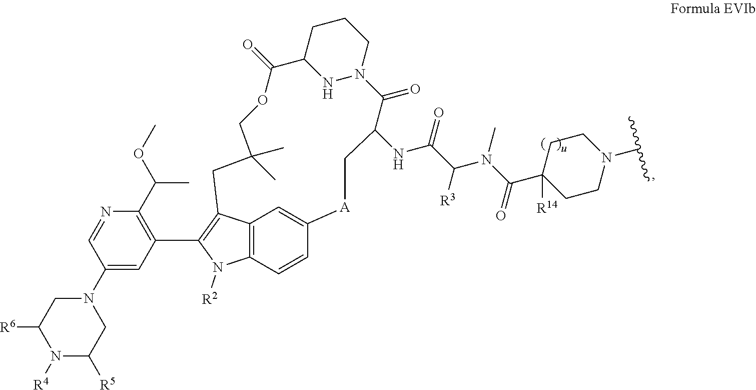

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl;
R¹⁴ is fluoro, hydrogen, or C₁-C₃ alkyl;
u is 0 or 1; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.

In some embodiments, the RAS(ON) inhibitor is provided as a conjugate, or salt thereof, comprising the structure of Formula EV:

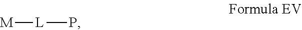

wherein L is a linker;
P is a monovalent organic moiety; and
M has the structure of Formula EVIc:

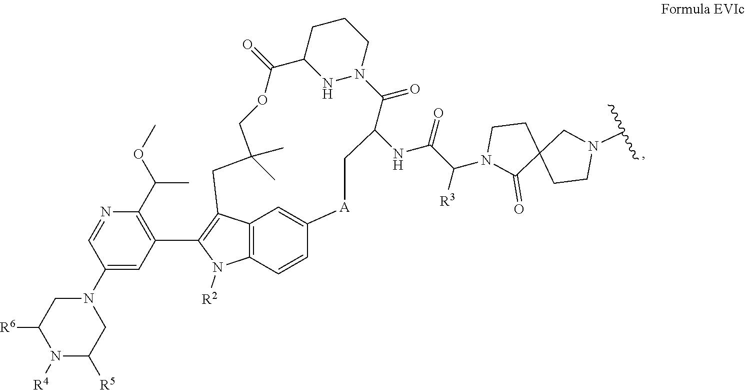

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.

In some embodiments, the RAS(ON) inhibitor is provided as a conjugate, or salt thereof, comprising the structure of Formula EV:

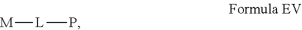

wherein L is a linker;
P is a monovalent organic moiety; and
M has the structure of Formula EVId:

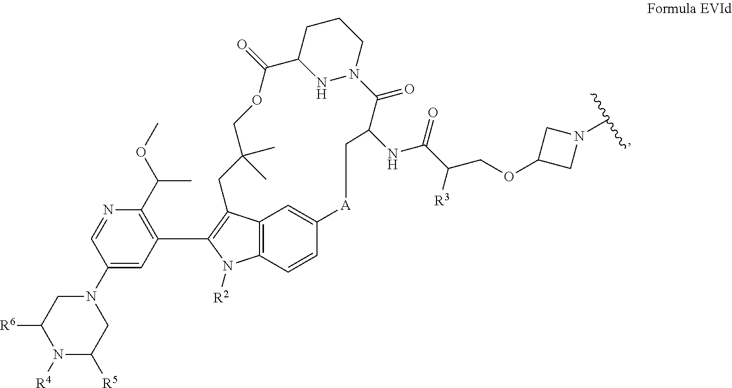

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
R² is optionally substituted C₁-C₆ alkyl;
R³ is optionally substituted C₁-C₆ alkyl or optionally substituted C₁-C₃ heteroalkyl; and
R⁴, R⁵, and R⁶ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁴ and R⁵ combine with the atoms to which they are attached to form an optionally substituted 3 to 8-membered cycloalkyl or an optionally substituted 3 to 8-membered heterocycloalkyl; or
R⁴ and R⁶ combine with the atoms to which they are attached to form an optionally substituted 3 to 8-membered cycloalkyl or an optionally substituted 3 to 8-membered heterocycloalkyl.

In some embodiments of conjugates of Formula EV, the monovalent organic moiety is a protein. In some embodiments, the protein is a Ras protein. In some embodiments, the Ras protein is K-Ras G12C, K-Ras G13C, H-Ras G12C, H-Ras G13C, N-Ras G12C, or N-Ras G13C. In some embodiments of conjugates of Formula EV, the linker is bound to the monovalent organic moiety through a bond to a sulfhydryl group of an amino acid residue of the monovalent organic moiety.

The compounds described herein in table E1 may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

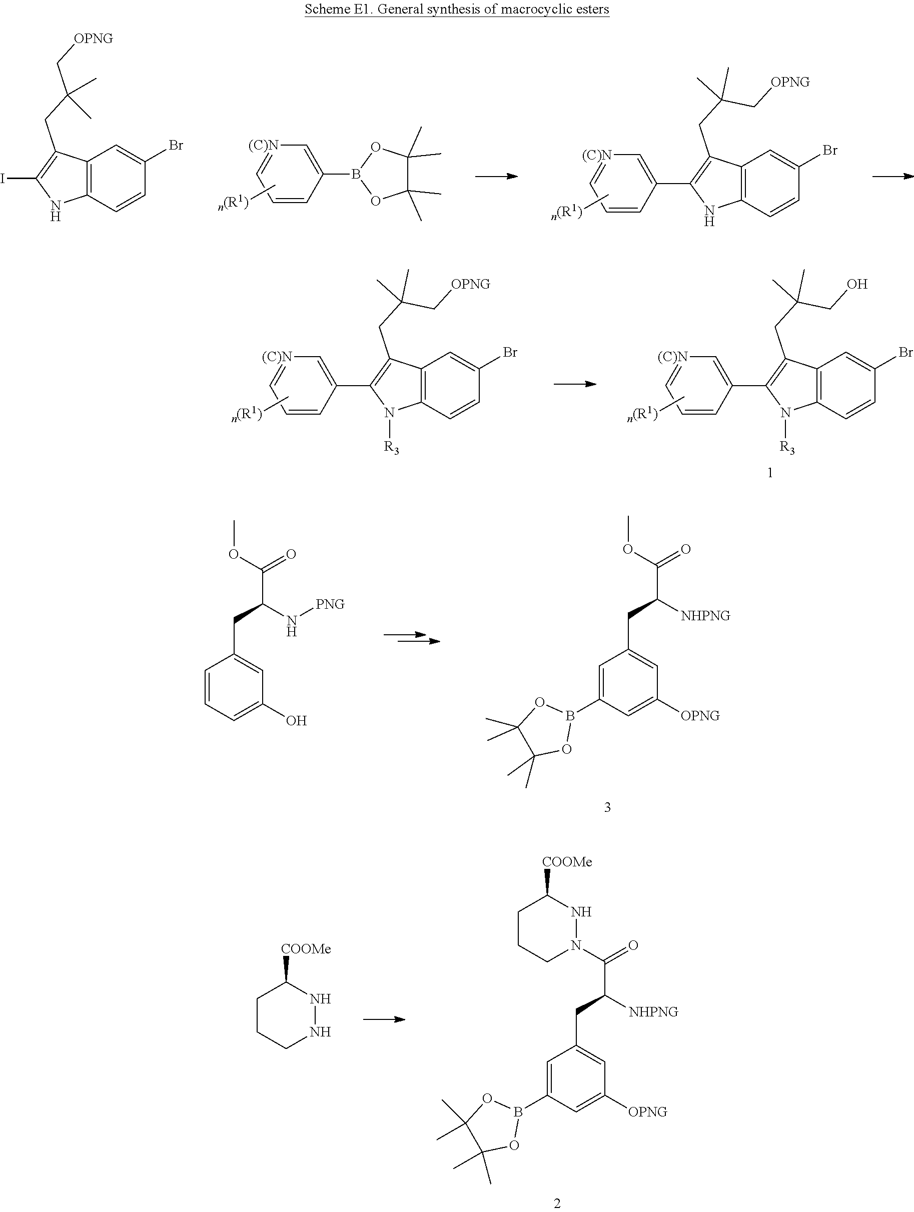

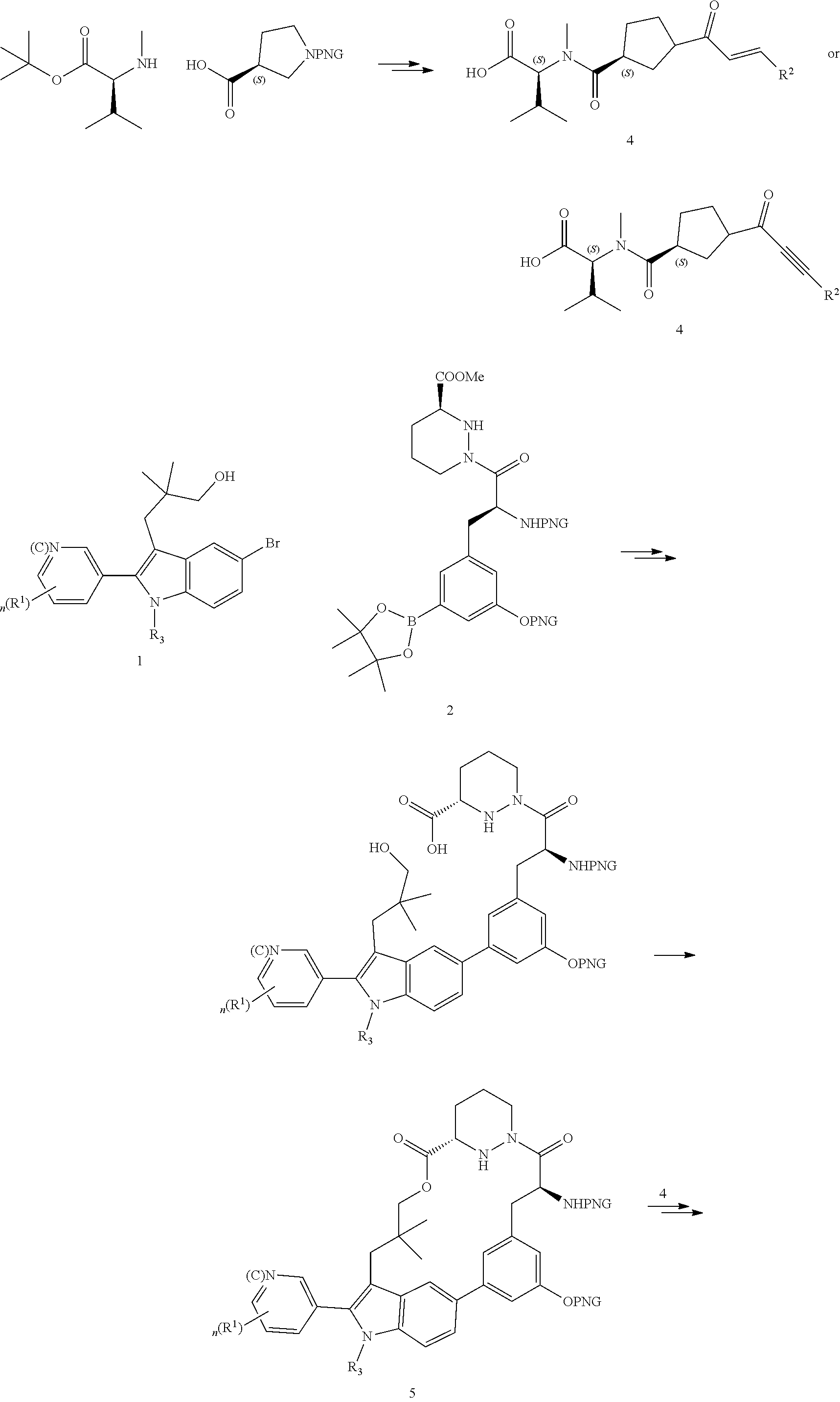

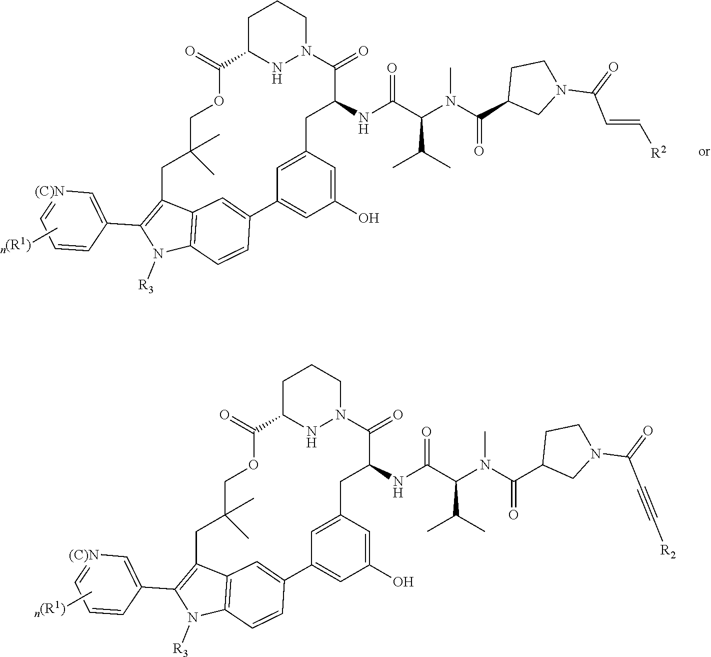

A general synthesis of macrocyclic esters is outlined in Scheme E1. An appropriately substituted aryl-3-(5-bromo-1-ethyl-1H-indol-3-yl)-2,2-dimethylpropan-1-ol (1) can be prepared in three steps starting from protected 3-(5-bromo-2-iodo-1H-indol-3-yl)-2,2-dimethylpropan-1-ol and appropriately substituted boronic acid, including palladium mediated coupling, alkylation, and de-protection reactions. Methyl-amino-hexahydropyridazine-3-carboxylate-boronic ester (2) can be prepared in three steps, including protection, iridium catalyst mediated borylation, and coupling with methyl (S)-hexahydropyridazine-3-carboxylate.

An appropriately substituted acetylpyrrolidine-3-carbonyl-N-methyl-L-valine (or an alternative aminoacid derivative (4) can be made by coupling of methyl-L-valinate and protected (S)-pyrrolidine-3-carboxylic acid, followed by deprotection, coupling with a carboxylic acid containing an appropriately substituted Michael acceptor, and a hydrolysis step.

The final macrocyclic esters can be made by coupling of methyl-amino-hexahydropyridazine-3-carboxylate-boronic ester (2) and aryl-3-(5-bromo-1-ethyl-1H-indol-3-yl)-2,2-dimethylpropan-1-ol (1) in the presence of a Pd catalyst followed by hydrolysis and macrolactonization steps to result in an appropriately protected macrocyclic intermediate (5). Deprotection and coupling with an appropriately substituted intermediate 4 results in a macrocyclic product. Additional deprotection or functionalization steps can be required to produce the final compound.

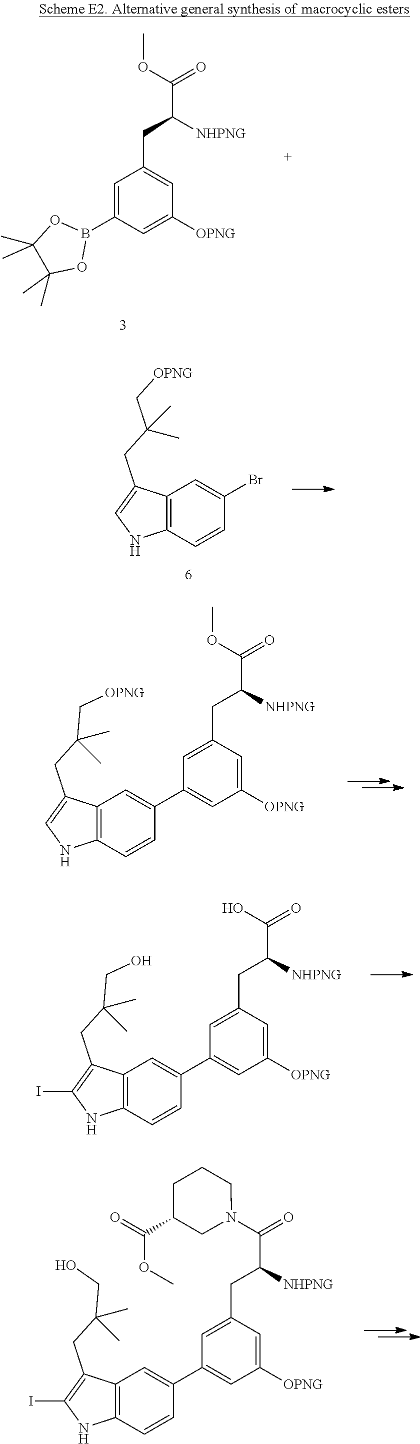

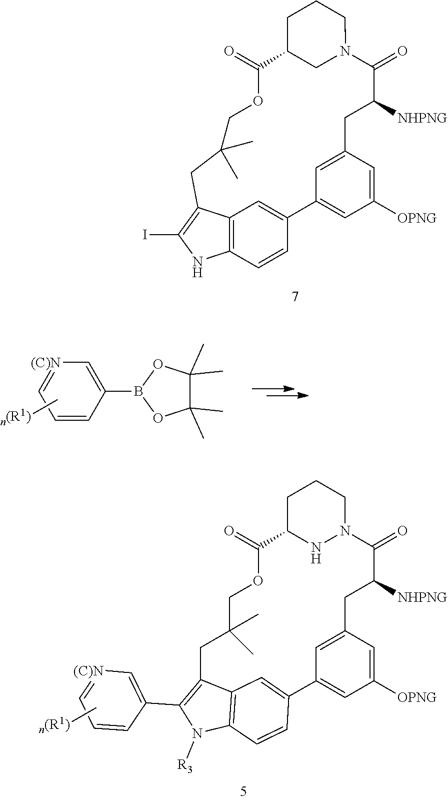

Alternatively, macrocyclic ester can be prepared as described in Scheme E2. An appropriately protected bromo-indolyl (6) coupled in the presence of a Pd catalyst with boronic ester (3), followed by iodination, deprotection, and ester hydrolysis. Subsequent coupling with methyl (S)-hexahydropyridazine-3-carboxylate, followed by hydrolysis and macrolactonization can result in iodo intermediate (7). Coupling in the presence of a Pd catalyst with an appropriately substituted boronic ester and alkyllation can yield fully protected macrocycle (5). Additional deprotection or functionalization steps are required to produce the final compound.

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below.

Compounds of Table E1 herein were prepared using methods disclosed herein or were prepared using methods disclosed herein combined with the knowledge of one of skill in the art. Compounds of Table E2 may be prepared using methods disclosed herein or may be prepared using methods disclosed herein combined with the knowledge of one of skill in the art.

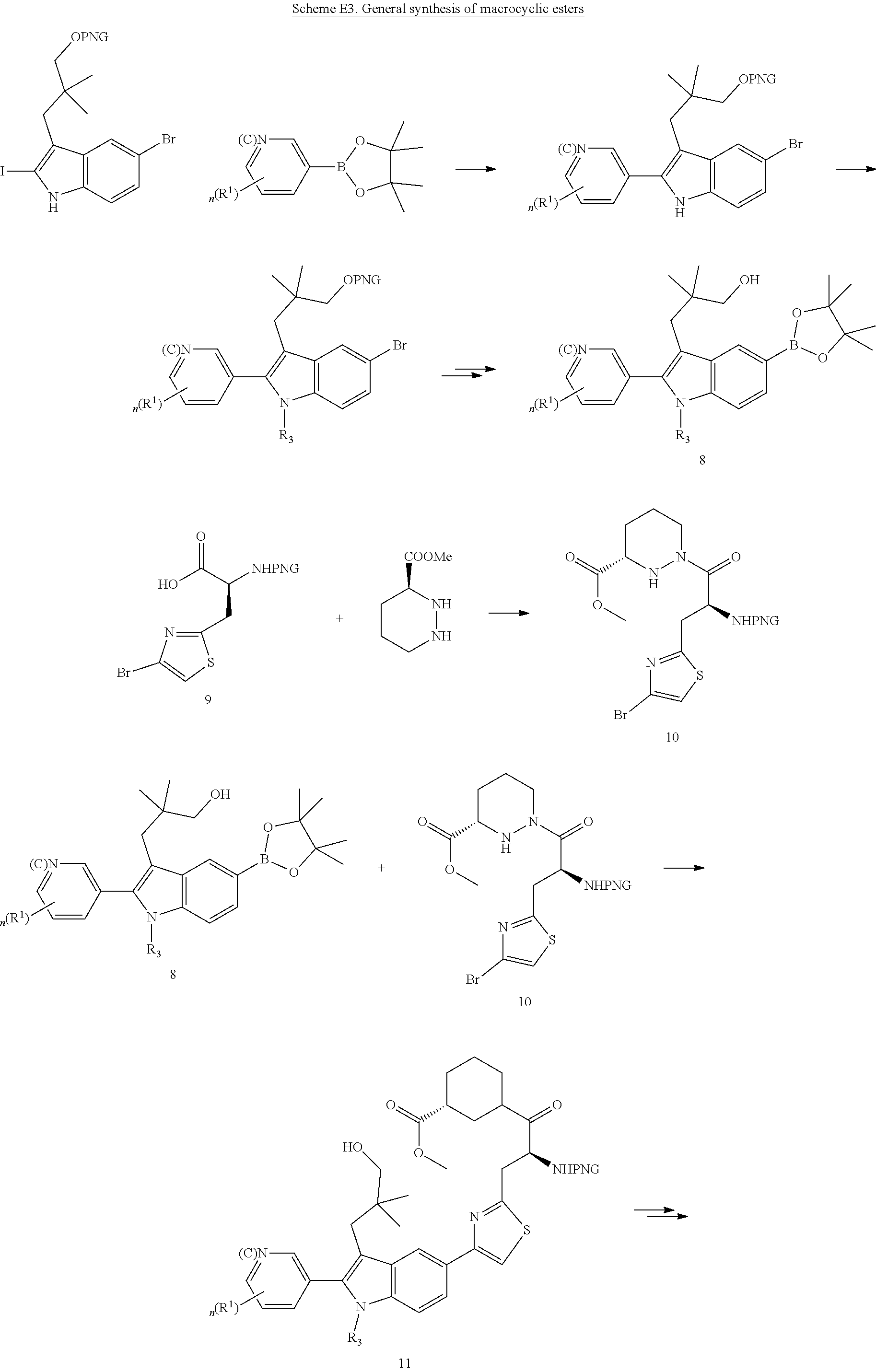

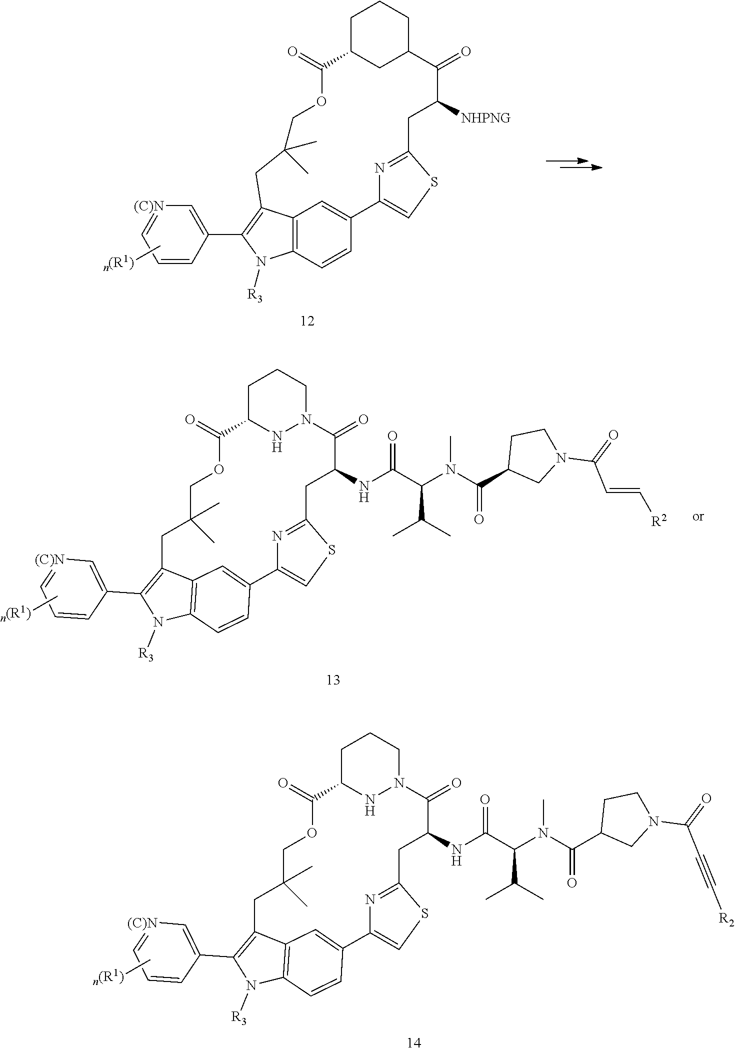

An alternative general synthesis of macrocyclic esters is outlined in Scheme E3. An appropriately substituted indolyl boronic ester (8) can be prepared in four steps starting from protected 3-(5-bromo-2-iodo-1H-indol-3-yl)-2,2-dimethylpropan-1-ol and appropriately substituted boronic acid, including Palladium mediated coupling, alkylation, de-protection, and Palladium mediated borylation reactions.

Methyl-amino-3-(4-bromothiazol-2-yl)propanoyl)hexahydropyridazine-3-carboxylate (10) can be prepared via coupling of (S)-2-amino-3-(4-bromothiazol-2-yl)propanoic acid (9) with methyl (S)-hexahydropyridazine-3-carboxylate.

The final macrocyclic esters can be made by coupling of Methyl-amino-3-(4-bromothiazol-2-yl)propanoyl)hexahydropyridazine-3-carboxylate (10) and an appropriately substituted indolyl boronic ester (8) in the presence of Pd catalyst followed by hydrolysis and macro lactonization steps to result in an appropriately protected macrocyclic intermediate (11). Deprotection and coupling with an appropriately substituted intermediate 4 can result in a macrocyclic product. Additional deprotection or functionalization steps could be required to produce a final compound 13 or 14.

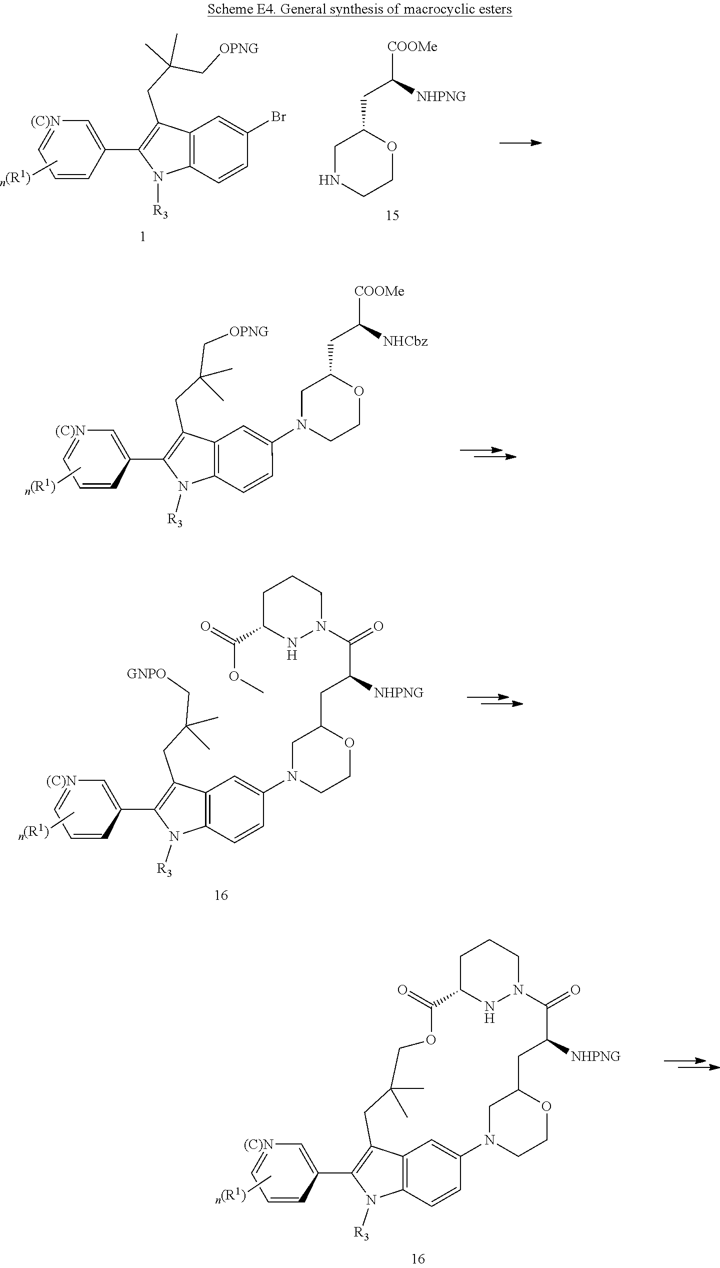

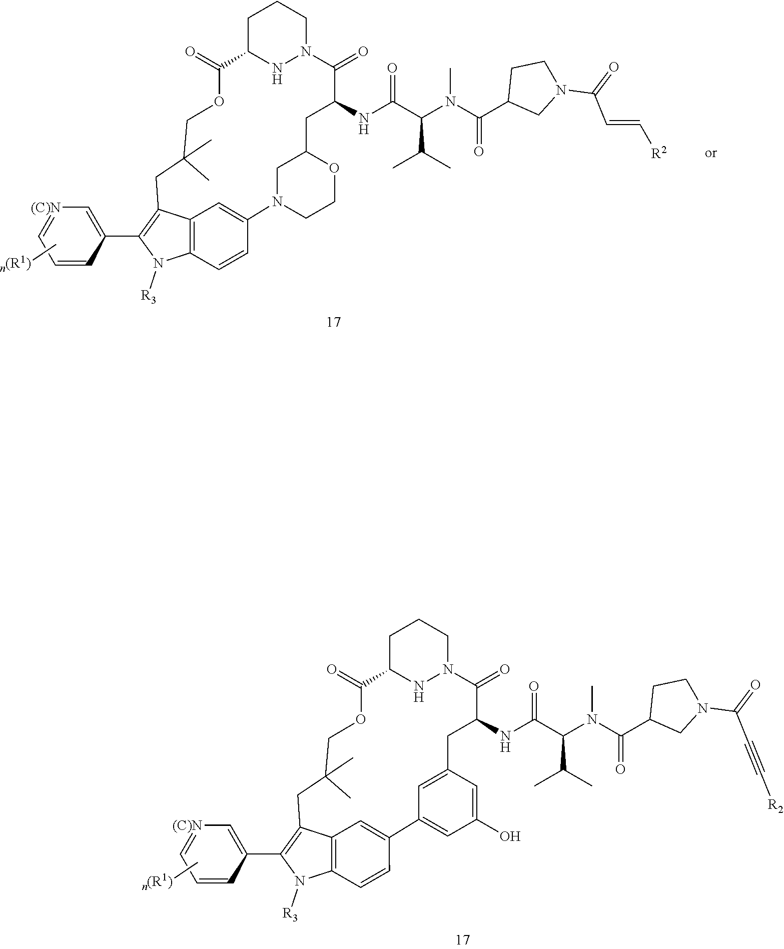

An alternative general synthesis of macrocyclic esters is outlined in Scheme E4. An appropriately substituted morpholine or an alternative herecyclic intermediate (15) can be coupled with appropriately protected Intermediate 1 via Palladium mediated coupling. Subsequent ester hydrolysis, and coupling with piperazoic ester results in intermediate 16.

The macrocyclic esters can be made by hydrolysis, deprotection and macrocyclization sequence. Subsequent deprotection and coupling with Intermediate 4 (or analogs) result in an appropriately substituted final macrocyclic products. Additional deprotection or functionalization steps could be required to produce a final compound 17.

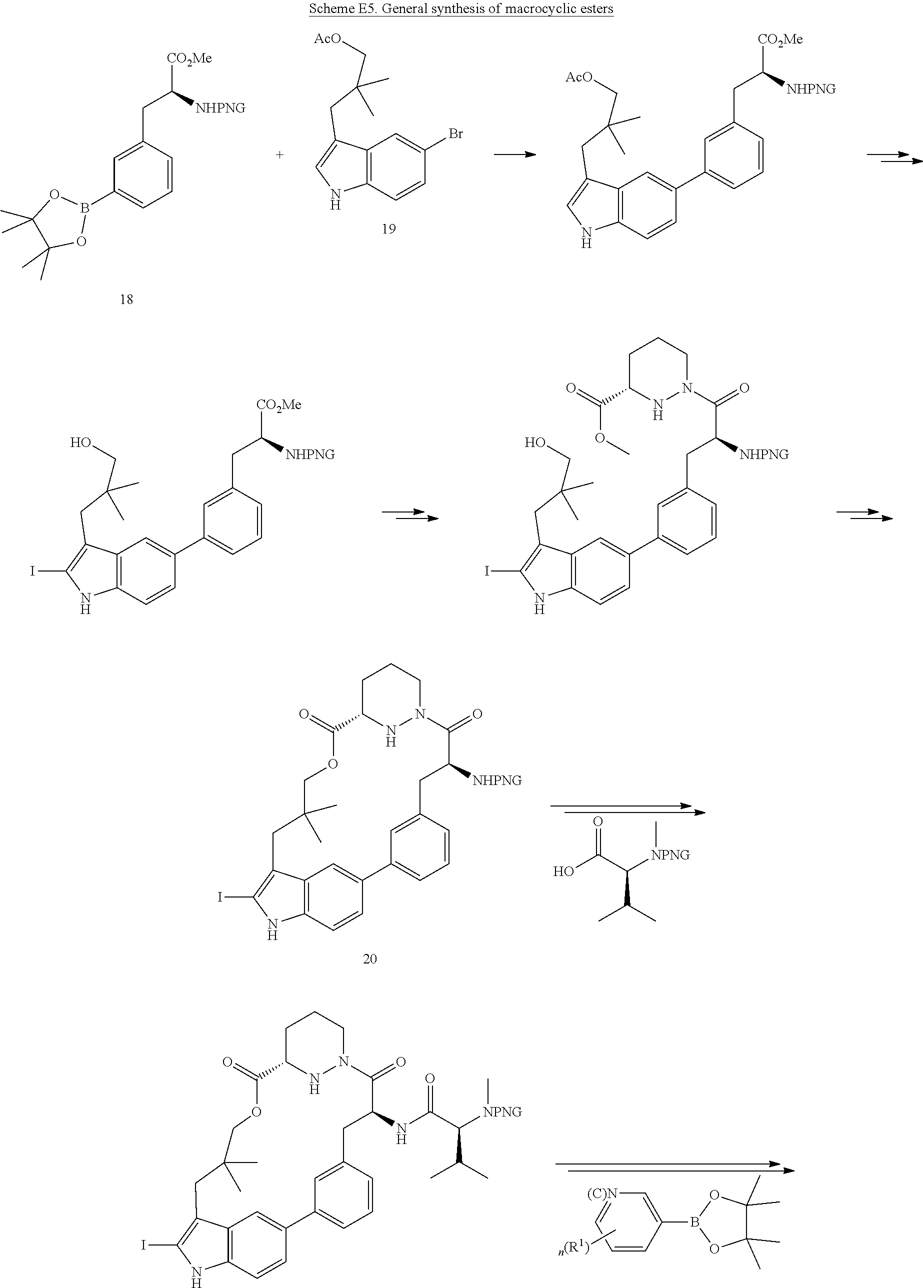

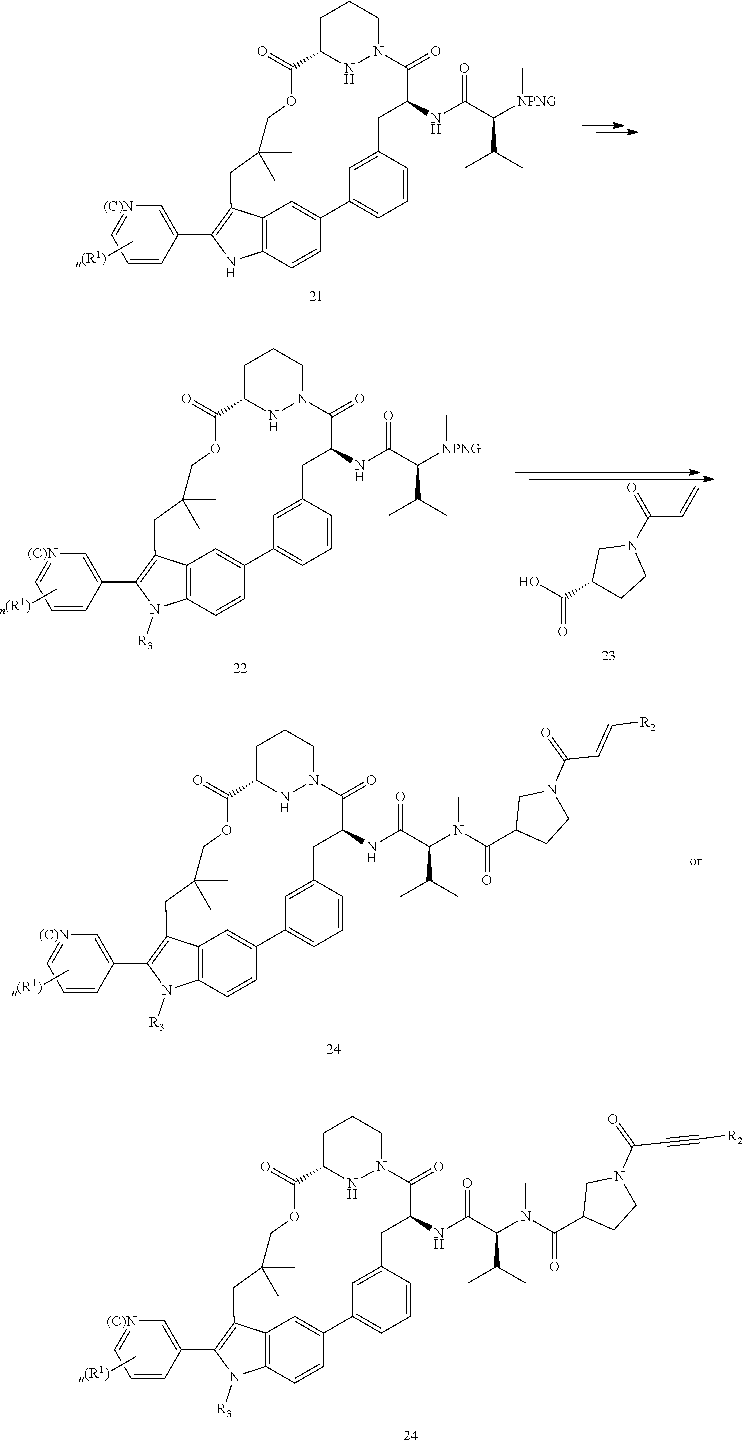

An alternative general synthesis of macrocyclic esters is outlined in Scheme E5. An appropriately substituted macrocycle (20) can be prepared starting from an appropriately protected boronic ester 18 and bromo indolyl intermediate (19), including Palladium mediated coupling, hydrolysis, coupling with piperazoic ester, hydrolysis, de-protection, and macrocyclizarion steps. Subsequent coupling with an appropriately substituted protected aminoacid followed by palladium mediated coupling yiels intermediate 21. Additional deprotection and derivatization steps, including alkyllation may be required at this point.

The final macrocyclic esters can be made by coupling of intermediate (22) and an appropriately substituted carboxylic acid intermediate (23). Additional deprotection or functionalization steps could be required to produce a final compound (24).

In addition, compounds of the disclosure can be synthesized using the methods described in the Examples below, together with synthetic methods known in the art of synthetic organic chemistry, or variations thereon as appreciated by those skilled in the art. These methods include but are not limited to those methods described in the Examples below.

In some embodiments, the RAS(ON) inhibitor is a compound, or pharmaceutically acceptable salt thereof, having the structure of Formula FI:

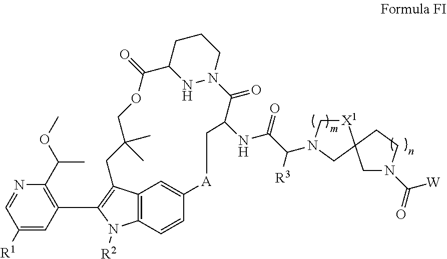

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
X¹ is CH₂ or O;
m is 1 or 2;
n is 0 or 1;
R¹ is hydrogen or optionally substituted 3 to 10-membered heterocycloalkyl;
R² is optionally substituted C₁-C₆ alkyl; and
R³ is optionally substituted C₁-C₆ alkyl or optionally substituted 3 to 6-membered cycloalkyl.

In some embodiments, W is a cross-linking group comprising an aziridine or an epoxide.

In some embodiments, A is optionally substituted thiazole, optionally substituted oxazole, optionally substituted morpholino, optionally substituted pyrrolidinyl, optionally substituted piperidinyl, or optionally substituted phenyl.

In some embodiments, the RAS(ON) inhibitor has the structure of Formula Fla, or a pharmaceutically acceptable salt thereof:

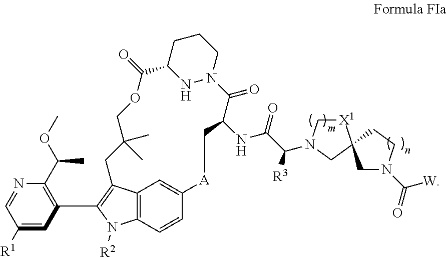

In some embodiments, the RAS(ON) inhibitor has the structure of Formula FII-1, or a pharmaceutically acceptable salt thereof:

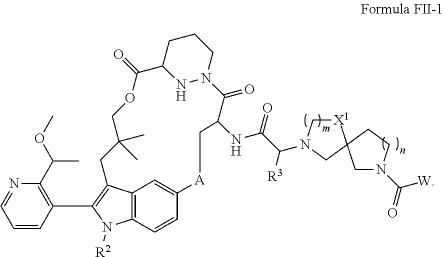

In some embodiments, the RAS(ON) inhibitor has the structure of Formula FII-2, or a pharmaceutically acceptable salt thereof:

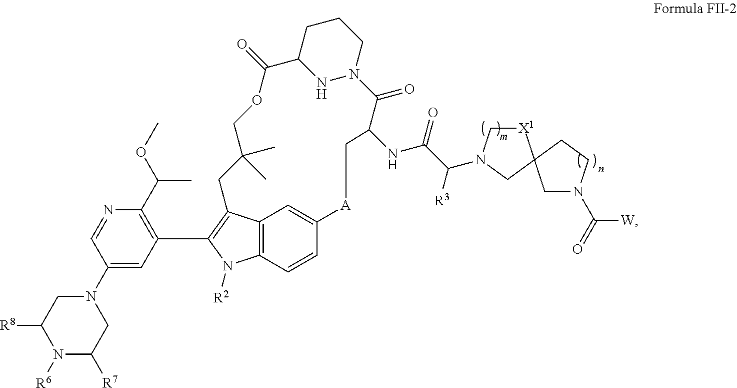

wherein R⁶, R′, and R⁸ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered heterocycloalkyl; or
R⁶ and R⁷ combine with the atoms to which they are attached to form an optionally substituted 3 to 8-membered cycloalkyl or an optionally substituted 3 to 8-membered heterocycloalkyl; or
R⁶ and R⁸ combine with the atoms to which they are attached to form an optionally substituted 3 to 8-membered cycloalkyl or an optionally substituted 3 to 8-membered heterocycloalkyl.

In some embodiments, the RAS(ON) inhibitor has the structure of Formula FII-3, or a pharmaceutically acceptable salt thereof:

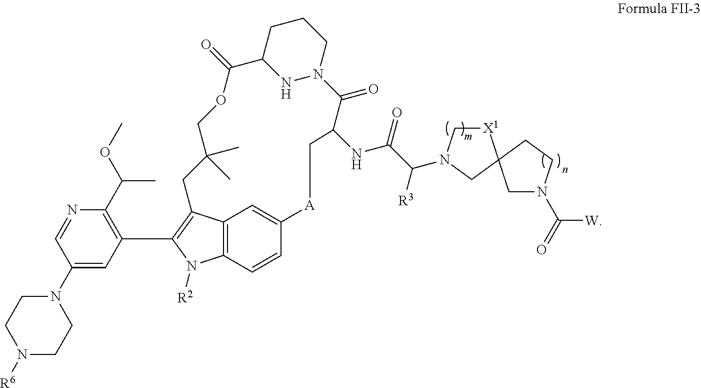

In some embodiments, the RAS(ON) inhibitor has the structure of Formula FII-4, or a pharmaceutically acceptable salt thereof:

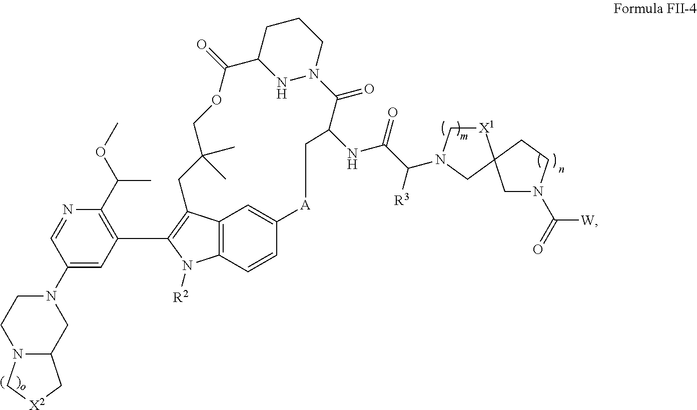

wherein X² is CH₂ or O; and
o is 1 or 2.

In some embodiments of Formula FI and subformula thereof, X² is CH₂. In some embodiments, o is 1. In some embodiments, o is 2.

In some embodiments of Formula FI and subformula thereof, X² is O. In some embodiments, o is 1. In some embodiments, o is 2.

In some embodiments of Formula FI and subformula thereof, R² is:

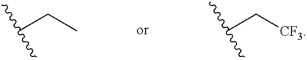

In some embodiments of Formula FI and subformula thereof, R³ is optionally substituted C₁-C₆ alkyl. In some embodiments, R³ is:

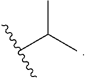 [SMILES: *C(C)C]

In some embodiments of Formula FI and subformula thereof, R³ is or optionally substituted 3 to 6-membered cycloalkyl. In some embodiments, R³ is:

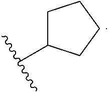 [SMILES: *C1CCCC1]

In some embodiments of Formula FI and subformula thereof, A is optionally substituted 5 to 10-membered heteroarylene. In some embodiments, A is:

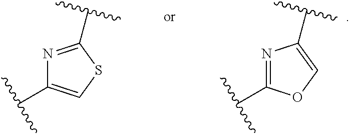 [SMILES: *c1csc(*)n1 ; *c1coc(*)n1]

In some embodiments of Formula FI and subformula thereof, A is optionally substituted phenyl. In some embodiments, A is:

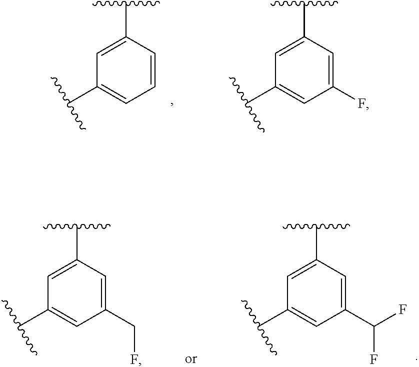

In some embodiments of Formula FI and subformula thereof, A is optionally substituted 3 to 6-membered heterocycloalkylene. In some embodiments, A is selected from the following, or a stereoisomer thereof:

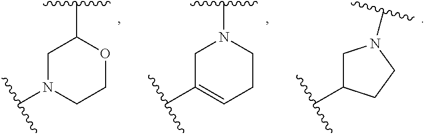 [SMILES: *C1CN(*)CCO1 ; *C1=CCCN(*)C1 ; *C1CCN(*)C1]

In some embodiments of Formula FI and subformula thereof, m is 1. In some embodiments, n is 1. In some embodiments, X¹ is CH₂. In some embodiments, X¹ is O. In some embodiments, m is 1, n is 1, and X¹ is CH₂. In some embodiments, m is 1, n is 1, and X¹ is O.

In some embodiments of Formula FI and subformula thereof, m is 2. In some embodiments, X¹ is CH₂. In some embodiments, n is 1. In some embodiments, n is 0. In some embodiments, m is 2, X¹ is CH₂, and n is 1. In some embodiments, m is 2 and X¹ is O. In some embodiments, m is 2, X¹ is O, and n is 1. In some embodiments, m is 2, X¹ is O, and n is 0.

In some embodiments of Formula FI and subformula thereof, W comprises an aziridine. In some embodiments, W comprises an optionally substituted cyclopropyl-aziridinyl moiety. In some embodiments, W is selected from the following, or a stereoisomer thereof:

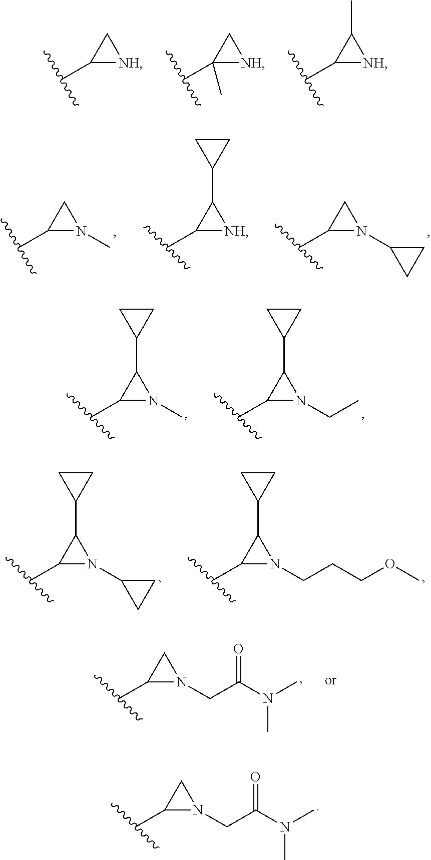

In some embodiments of Formula FI and subformula thereof, W comprises an epoxide. In some embodiments, W is selected from the following, or a stereoisomer thereof:

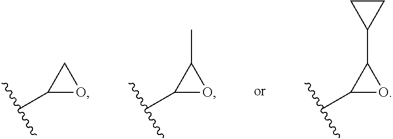

In some embodiments, the RAS(ON) inhibitor is selected from Table F1, or a pharmaceutically acceptable salt or stereoisomer thereof. In some embodiments, a compound of the present invention is selected from Table F1, or a pharmaceutically acceptable salt or atropisomer thereof.

TABLE F1
Certain Compounds of the Present Invention

| Ex # | Structure |
|---|---|
| FA 1 | 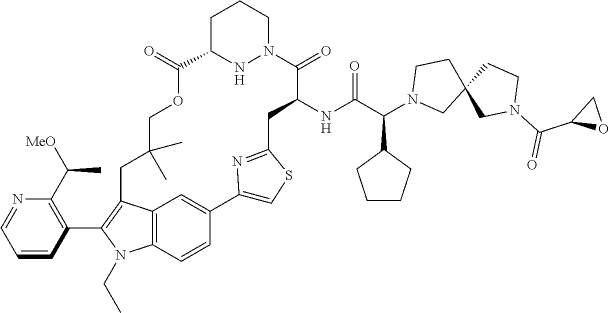 |
| FA 2 | 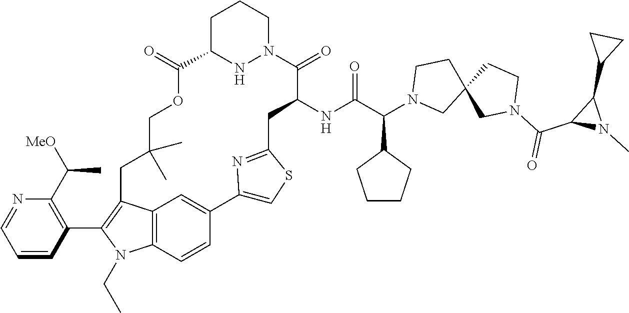 |
| FA 3 | 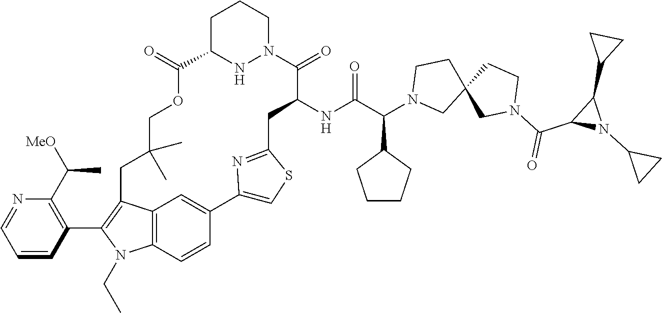 |
| FA 4 | 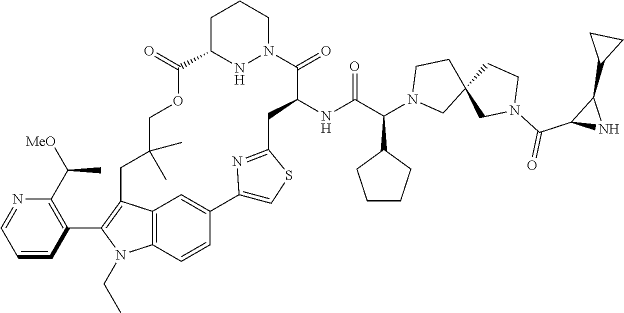 |
| FA 5 | 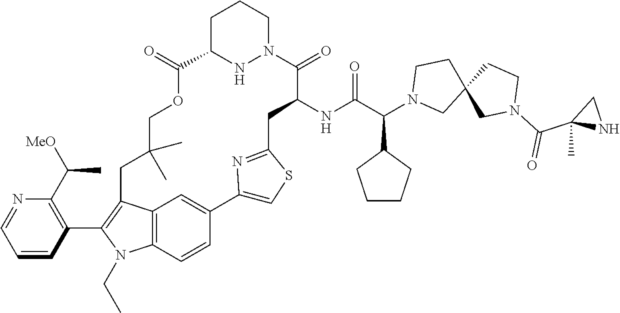 |
| FA 6 | 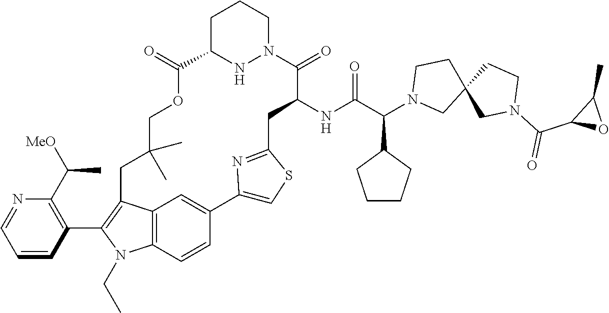 |
| FA 7 | 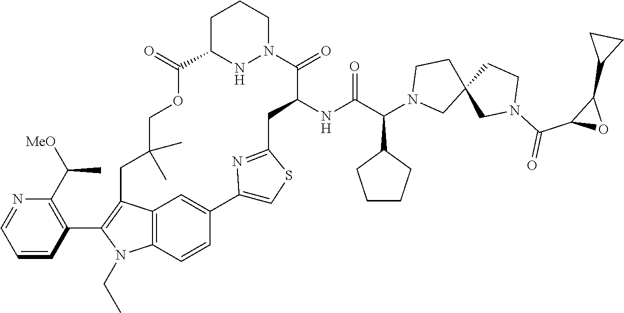 |
| FA 8 | 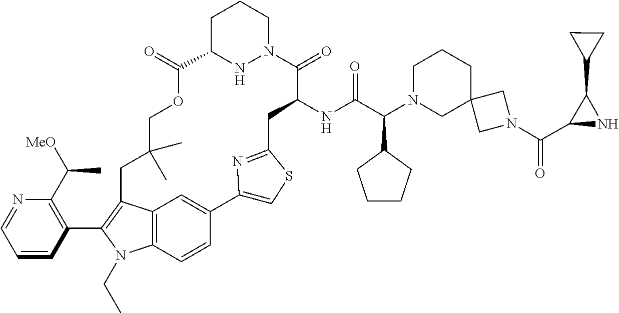 |
| FA 9 | 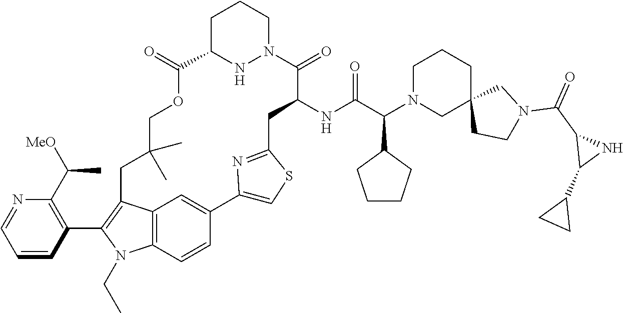 |
| FA 10 | 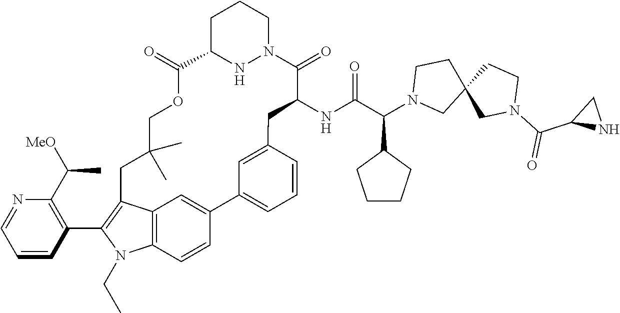 |
| FA 11 | 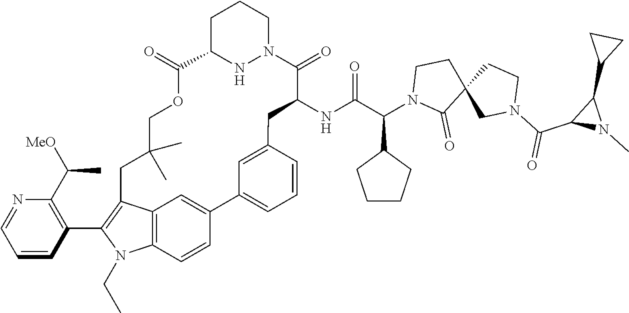 |
| FA 12 | 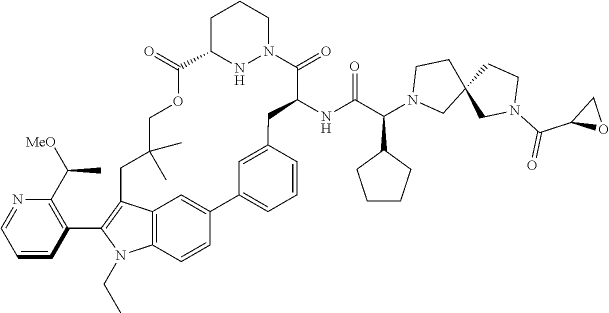 |
| FA 13 | 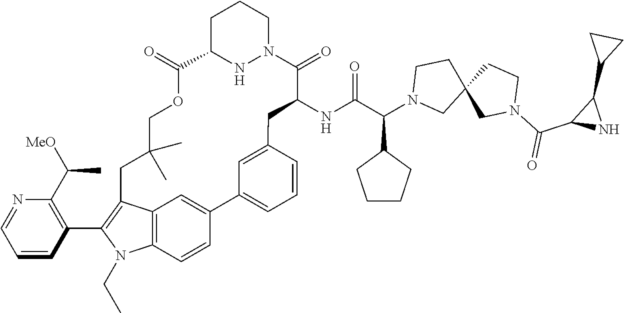 |
| FA 14 | 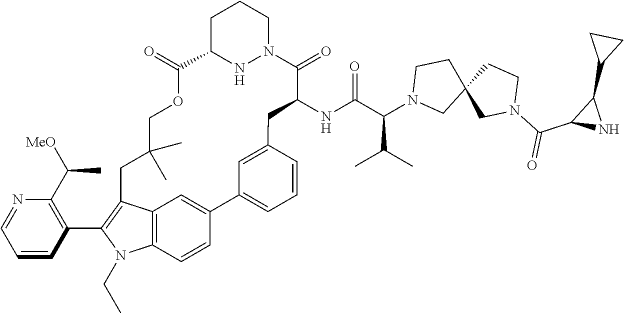 |
| FA 15 | 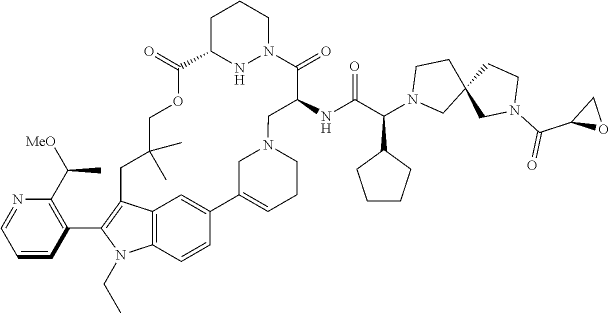 |
| FA 16 | 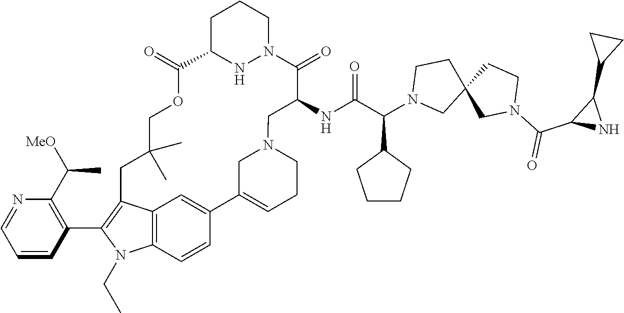 |
| FA 17 | 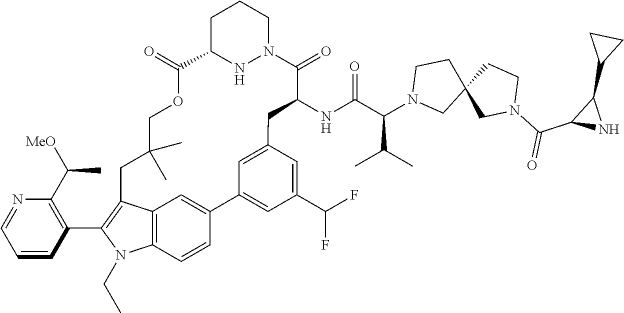 |
| FA 18 | 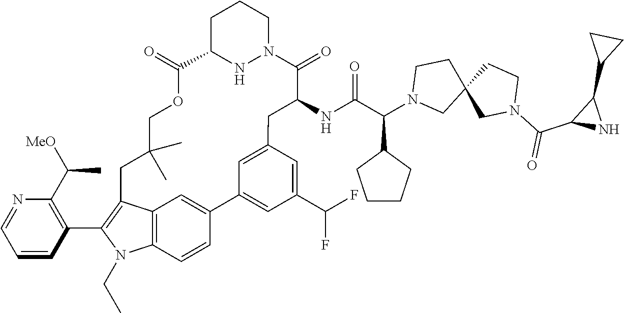 |
| FA 19 | 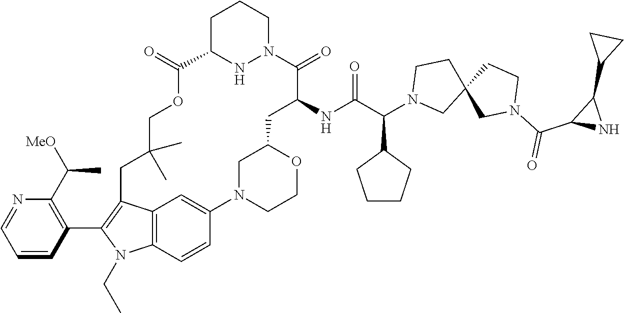 |
| FA 20 | 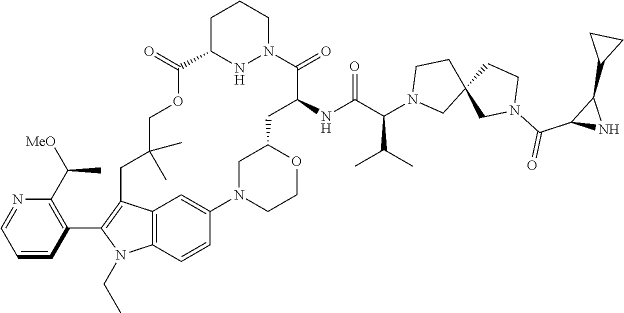 |
| FA 21 | 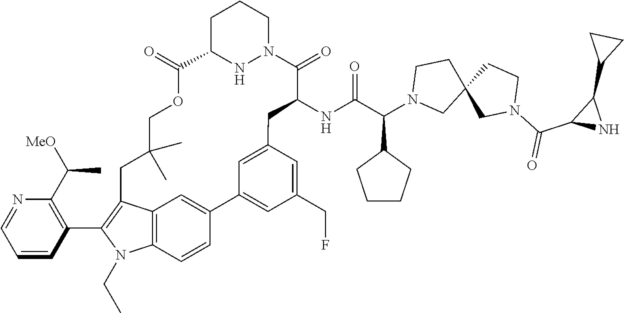 |
| FA 22 | 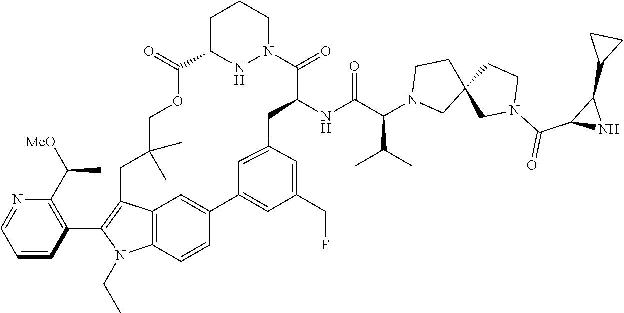 |
| FA 23 | 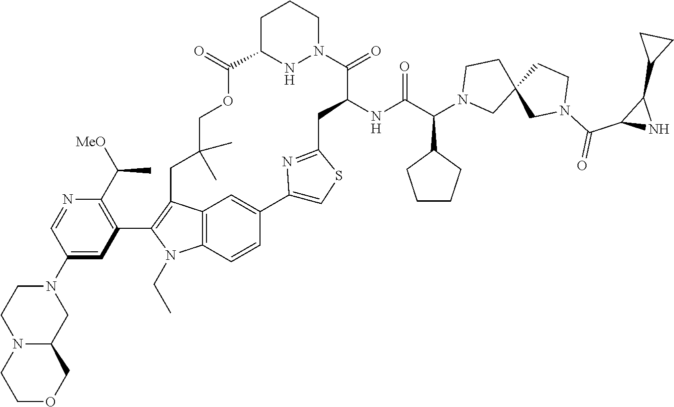 |
| FA 24 | 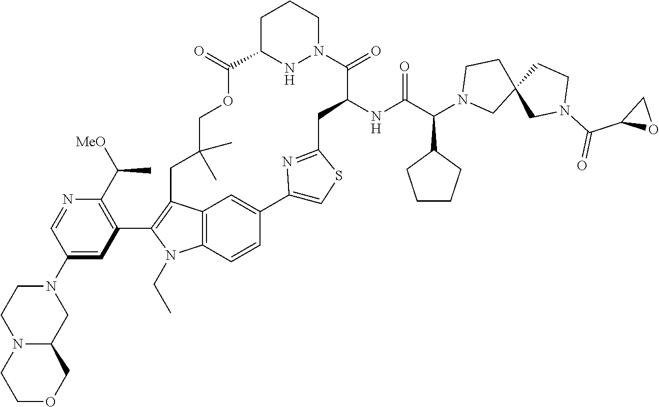 |
| FA 25 | 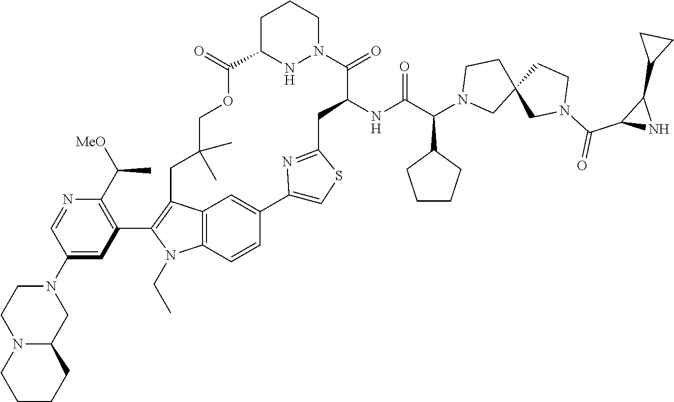 |
| FA 26 | 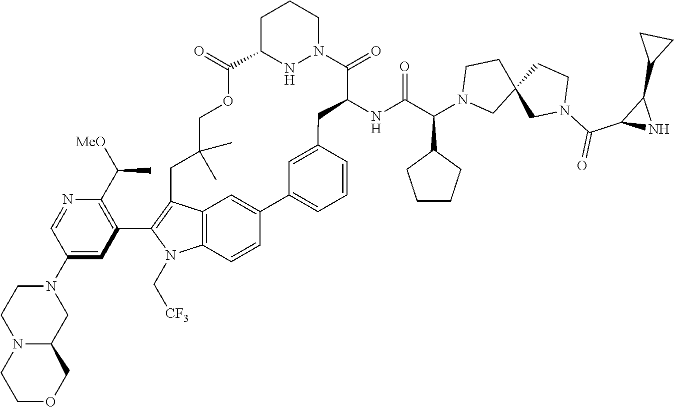 |
| FA 27 | 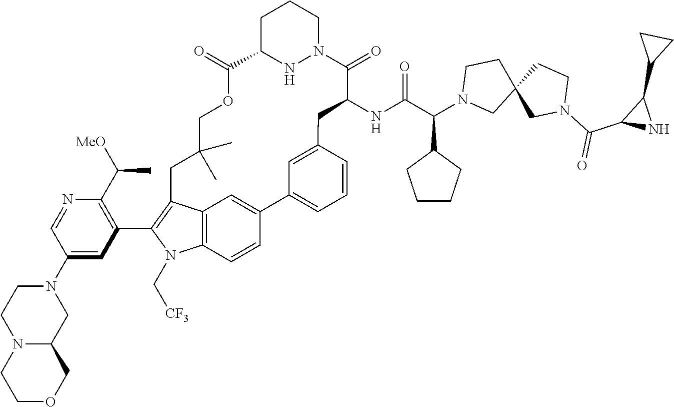 |
| FA 28 | 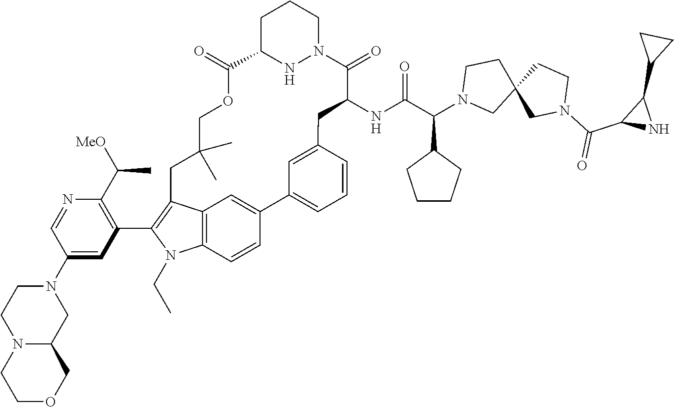 |
| FA 29 | 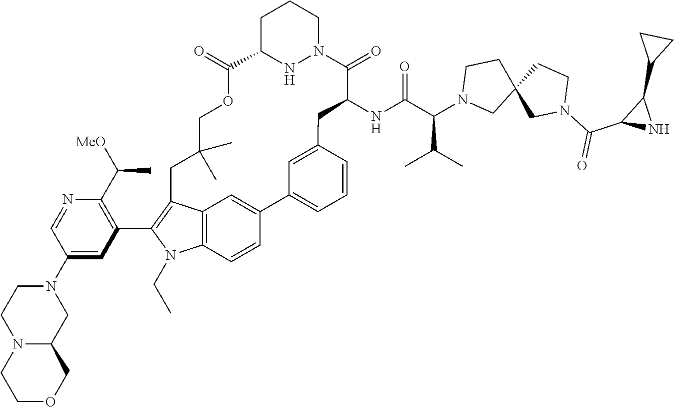 |
| FA 30 | 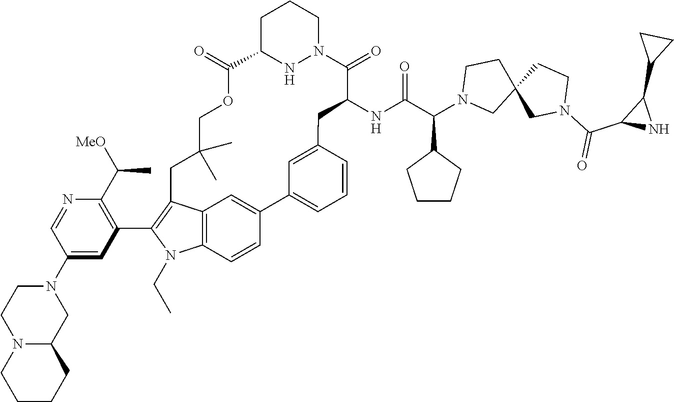 |
| FA 31 | 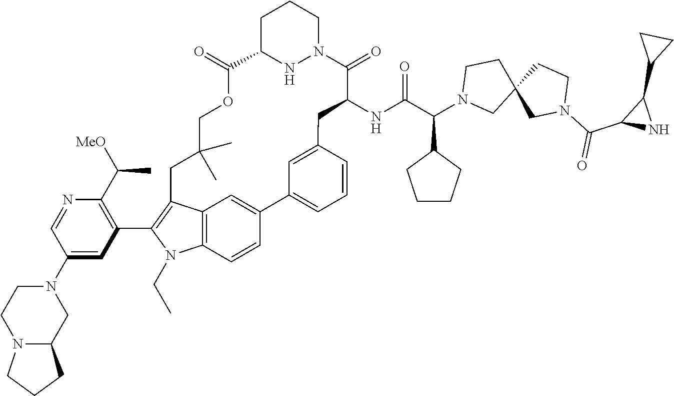 |
| FA 32 | 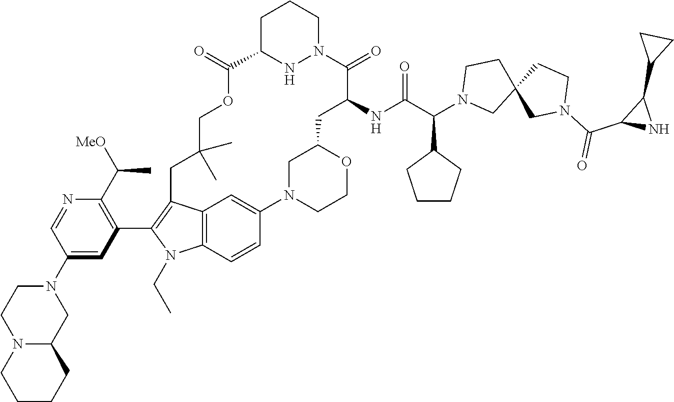 |
| FA 33 | 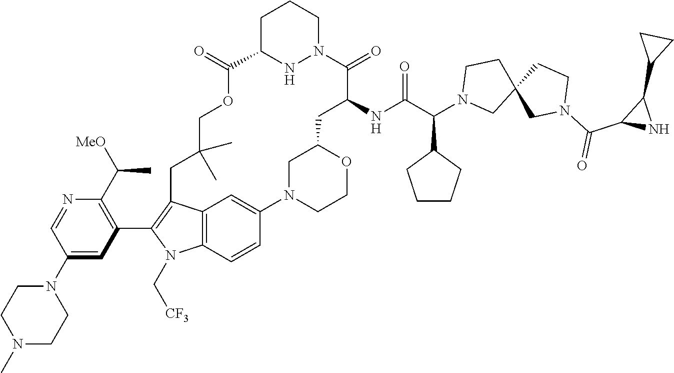 |
| FA 34 | 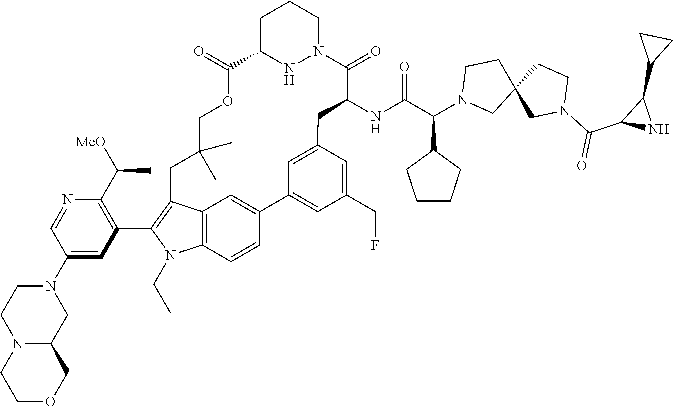 |
| FA 35 | 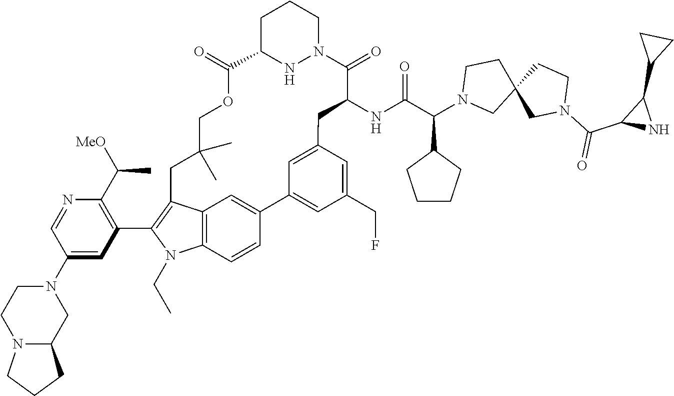 |
| FA 36 | 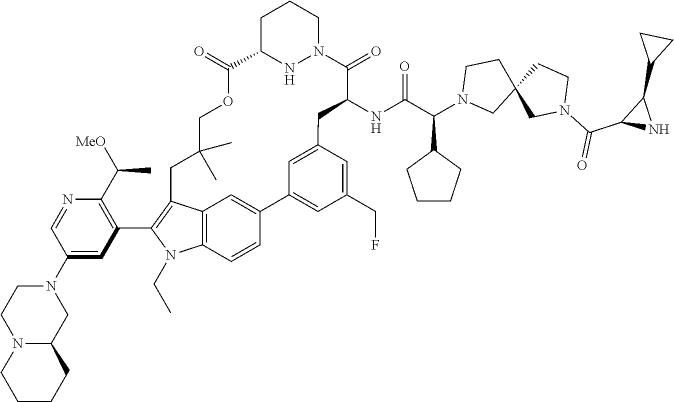 |
| FA 37 | 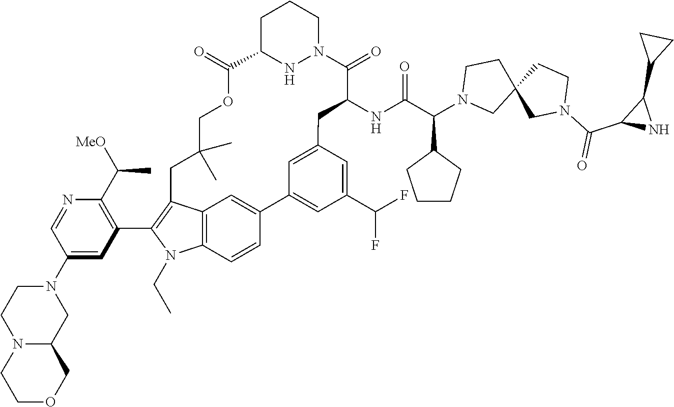 |
| FA 38 | 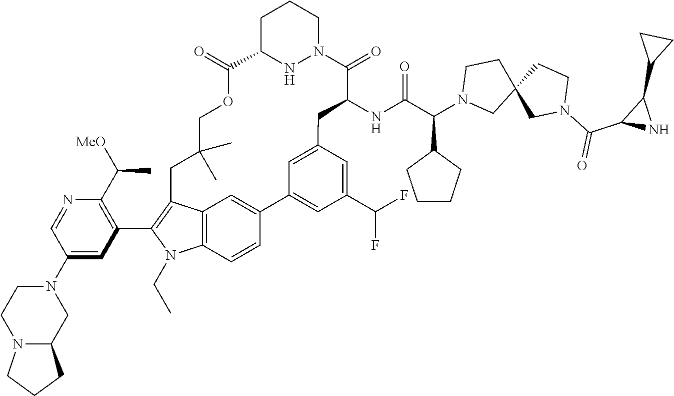 |
| FA 39 | 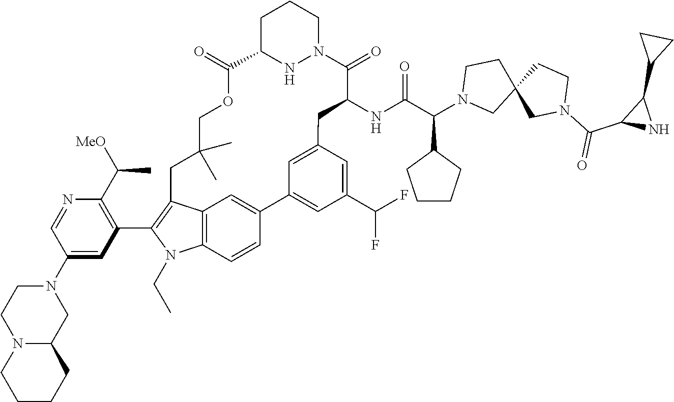 |
| FA 40 | 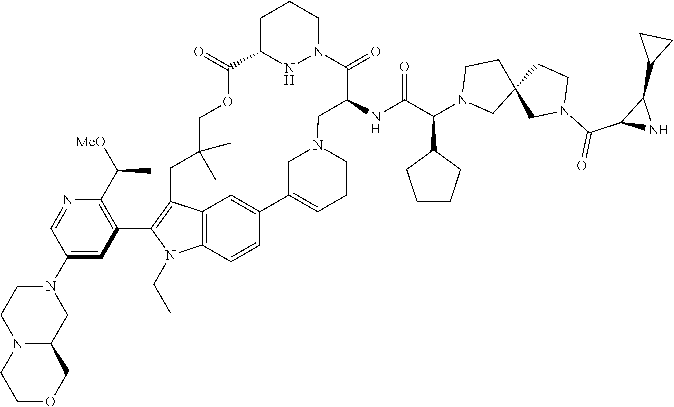 |
| FA 41 | 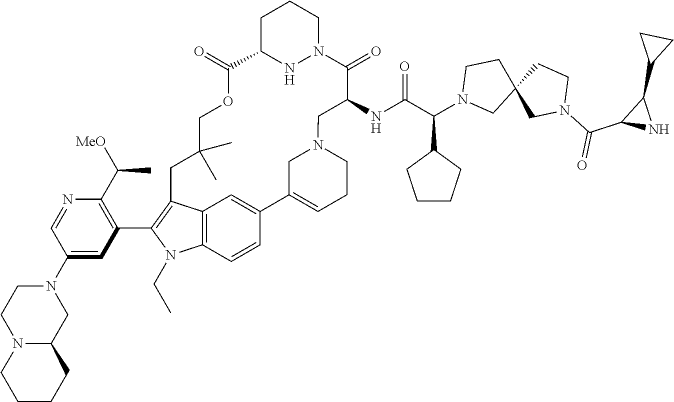 |
| FA 42 | 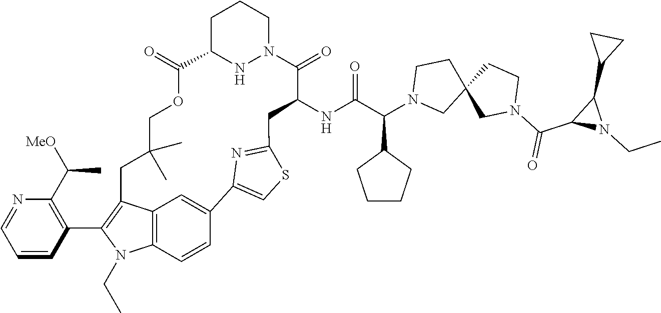 |
| FA 43 | 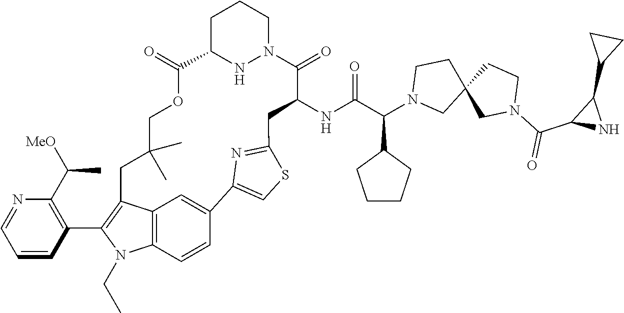 |
| FA 44 | 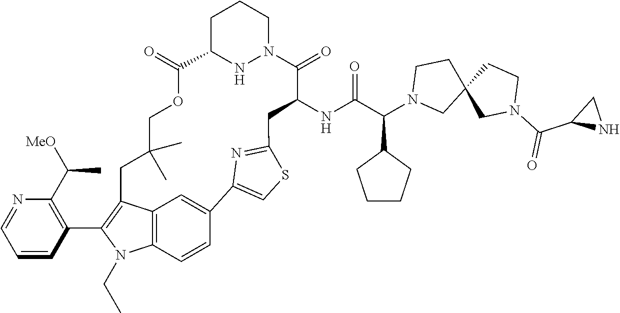 |

* Stereochemistry of the aziridine carbon is assumed.

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated.

In some embodiments, the RAS(ON) inhibitor is selected from Table F2, or a pharmaceutically acceptable salt thereof. In some embodiments, the RAS(ON) inhibitor is selected from Table F2, or a pharmaceutically acceptable salt or atropisomer thereof.

TABLE F2
Certain Compounds of the Present Invention

| Ex # | Structure |
|---|---|
| FB 1 | 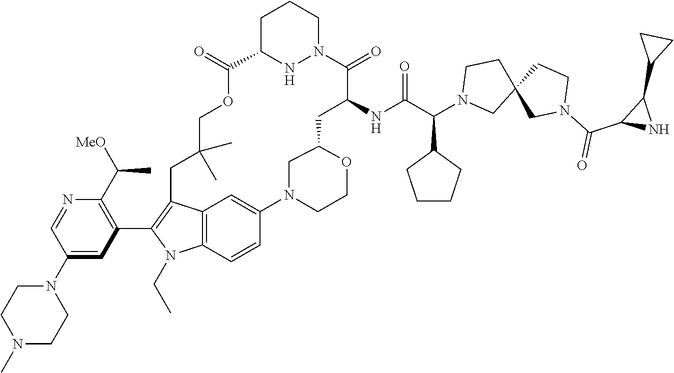 |
| FB 2 | 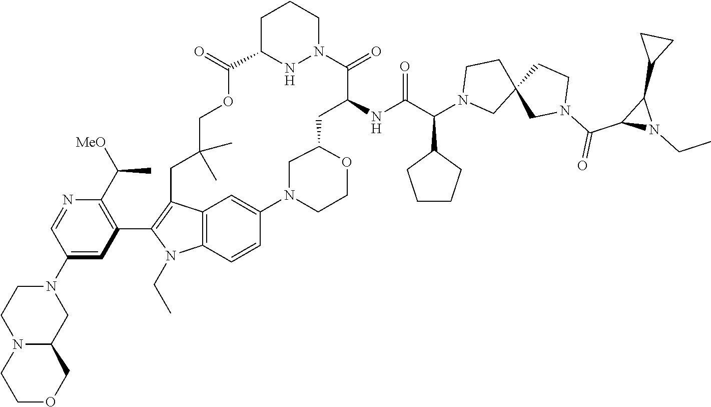 |
| FB 3 | 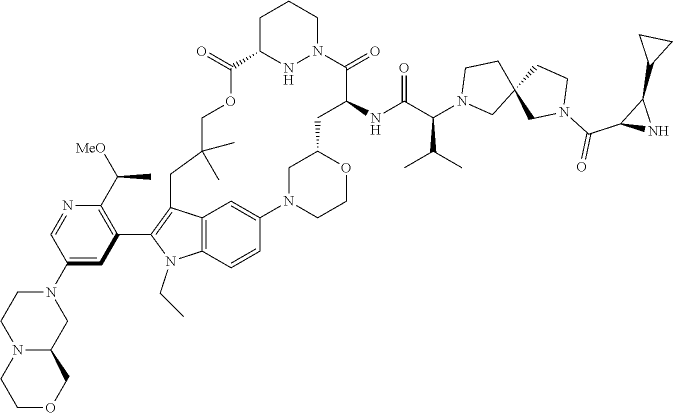 |
| FB 4 | 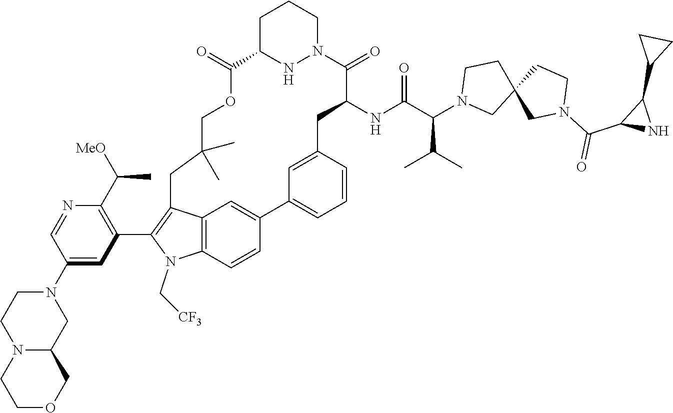 |
| FB 5 | 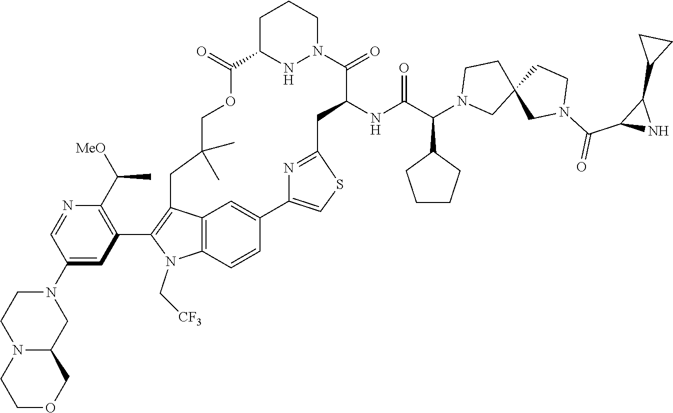 |
| FB 6 | 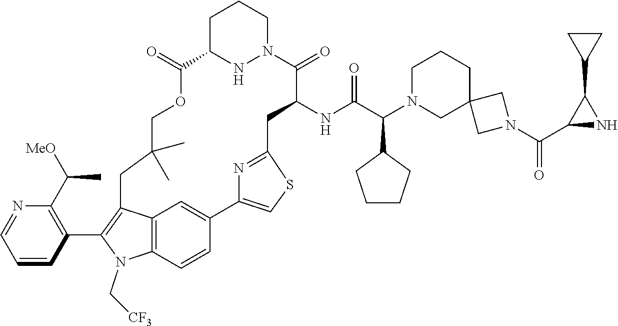 |
| FB 7 | 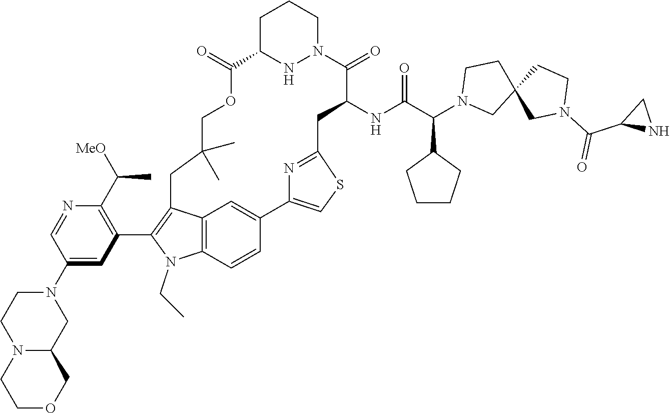 |
| FB 8 | 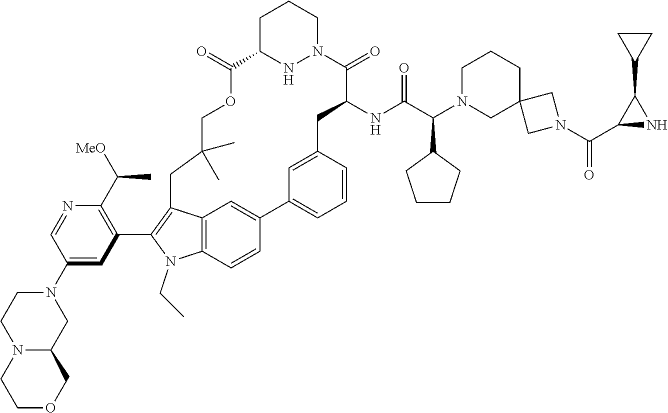 |
| FB 9 | 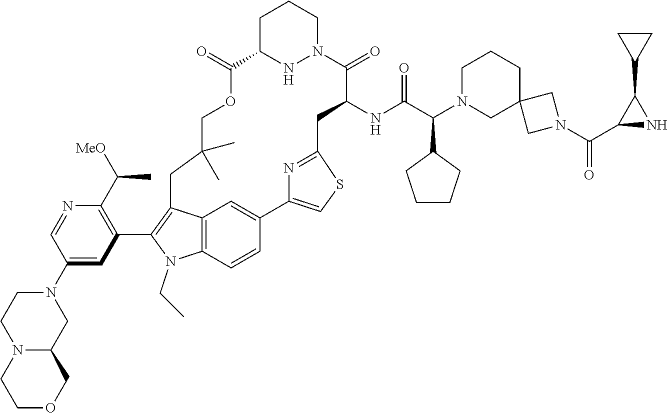 |
| FB 10 | 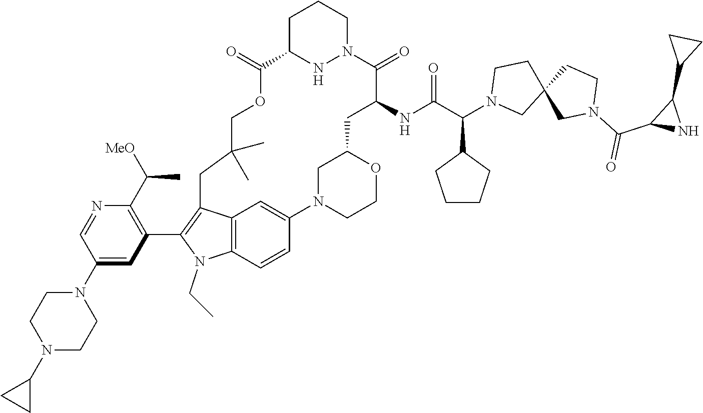 |
| FB 11 | 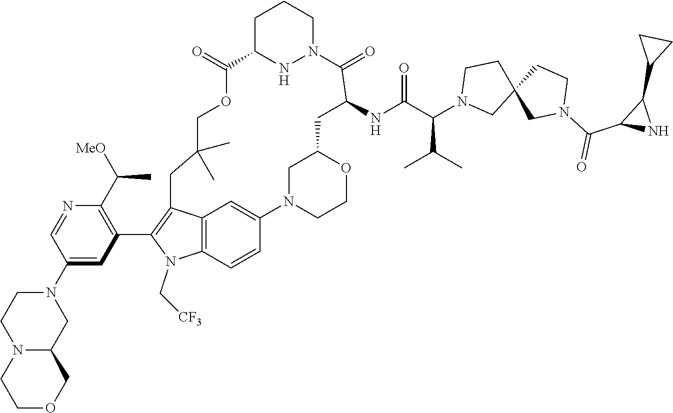 |
| FB 12 | 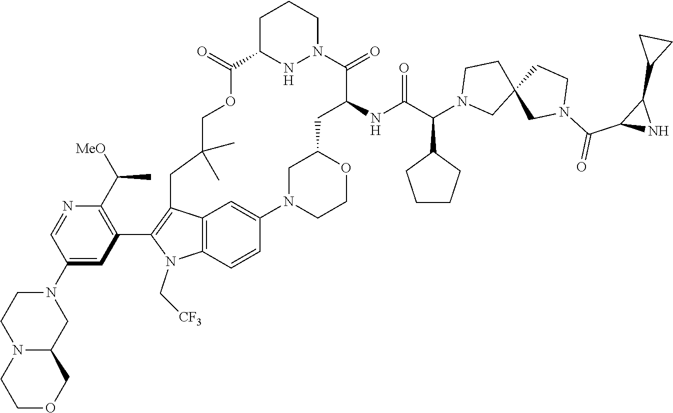 |
| FB 13 | 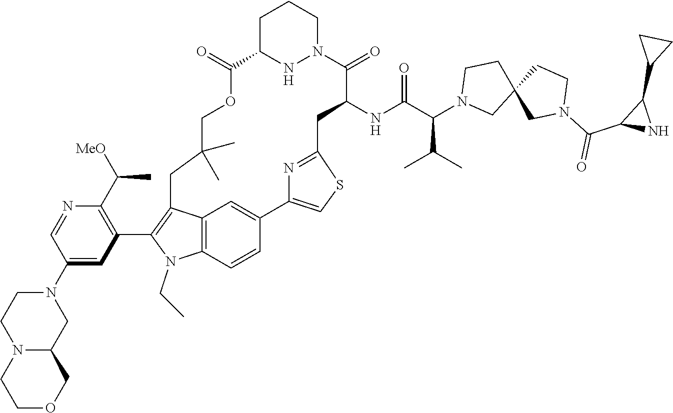 |
| FB 14 | 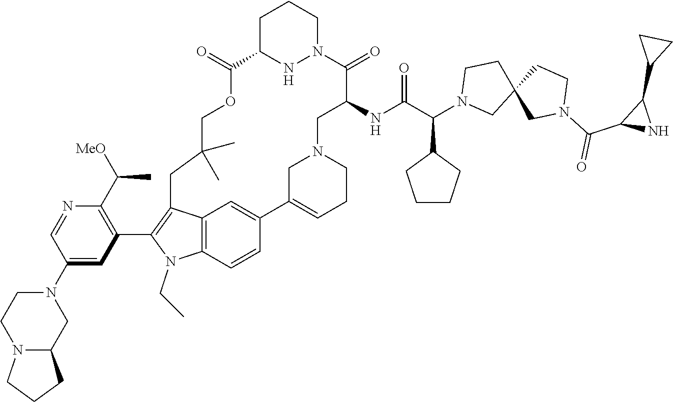 |
| FB 15 | 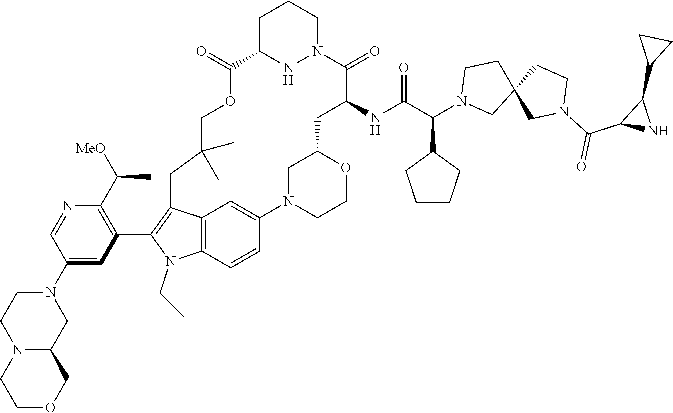 |
| FB 16 | 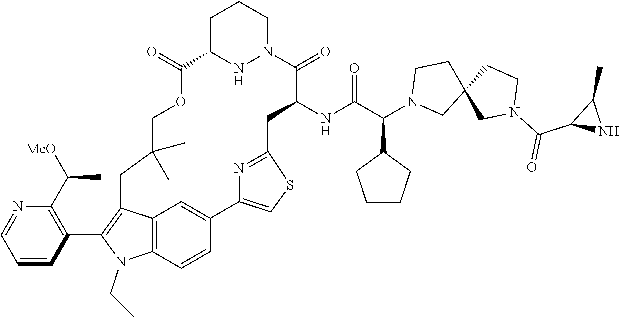 |
| FB 17 | 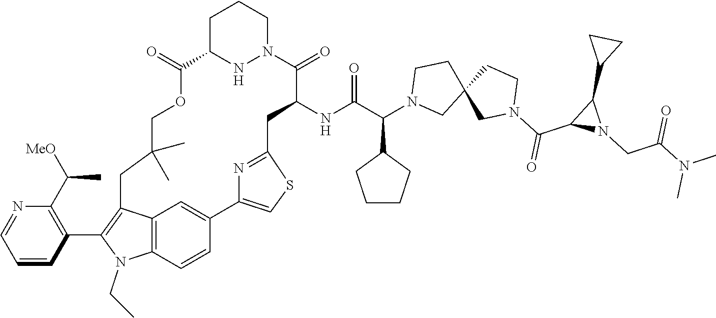 |
| FB 18 | 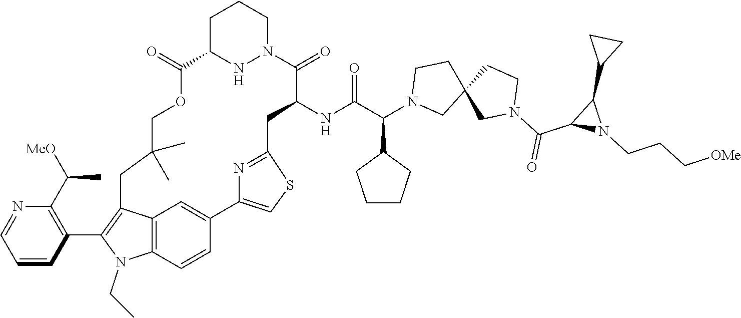 |
| FB 19 | 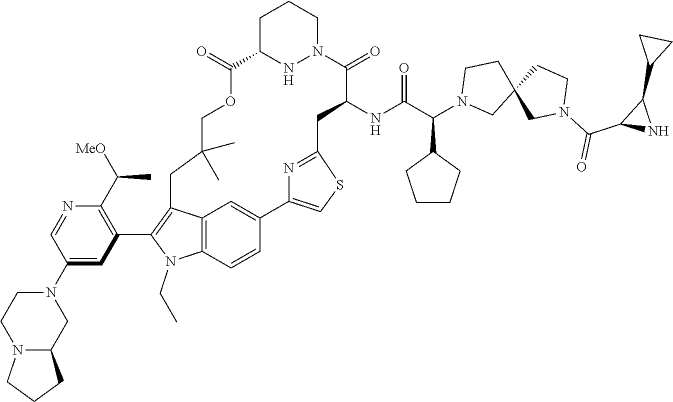 |
| FB 20 | 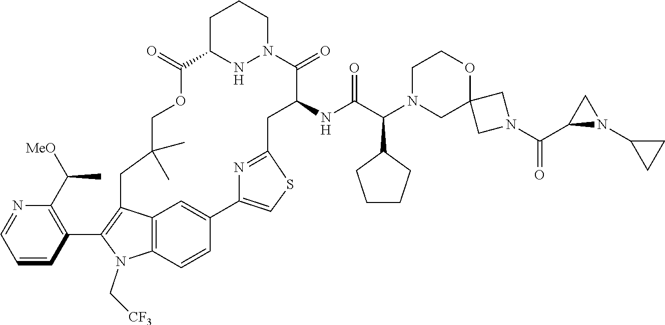 |
| FB 21 | 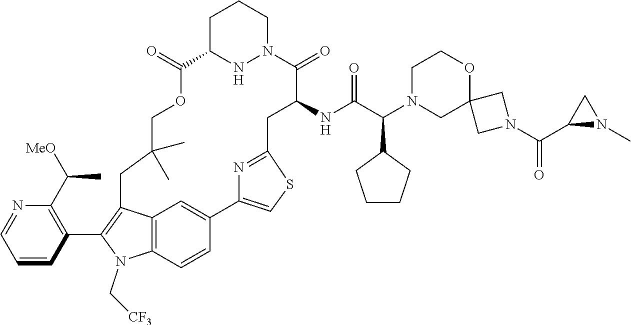 |
| FB 22 | 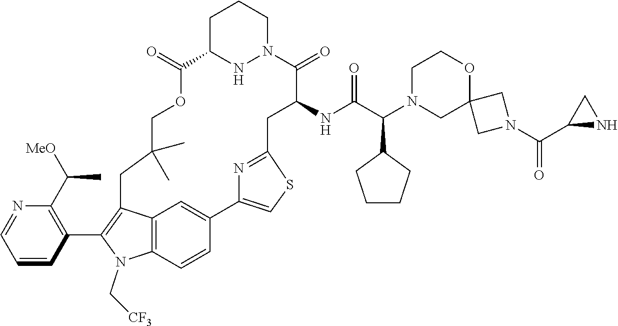 |
| FB 23 | 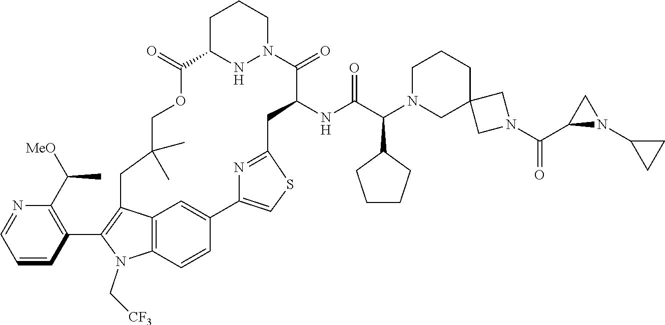 |
| FB 24 | 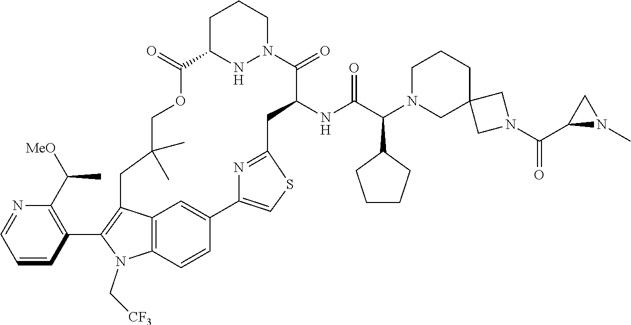 |
| FB 25 | 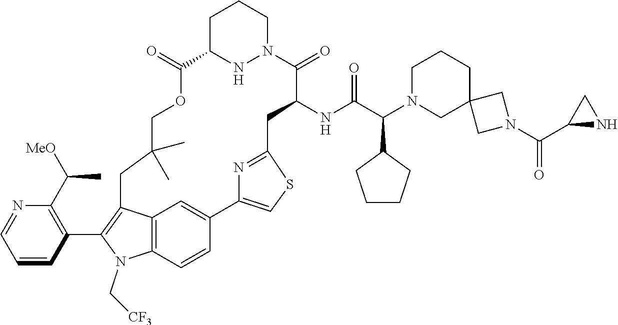 |
| FB 26 | 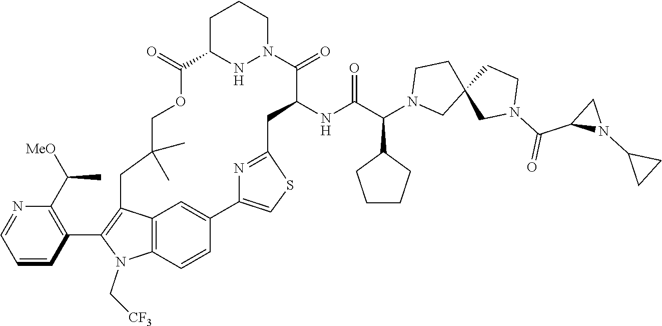 |
| FB 27 | 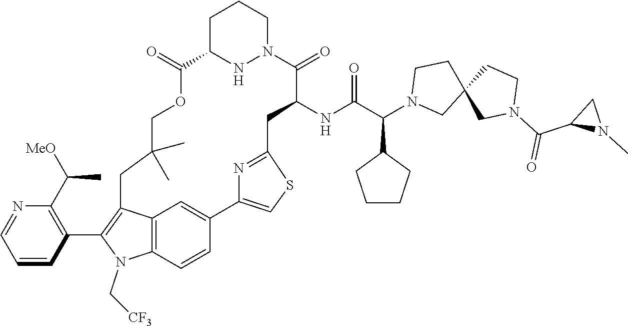 |
| FB 28 | 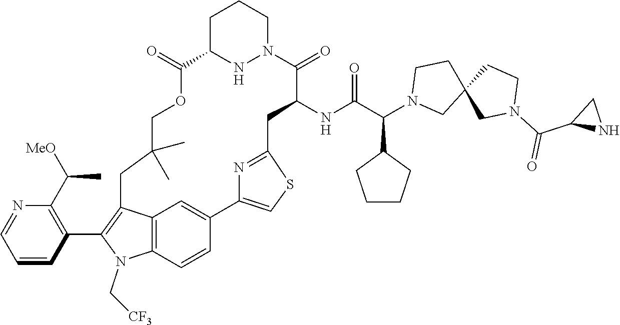 |
| FB 29 | 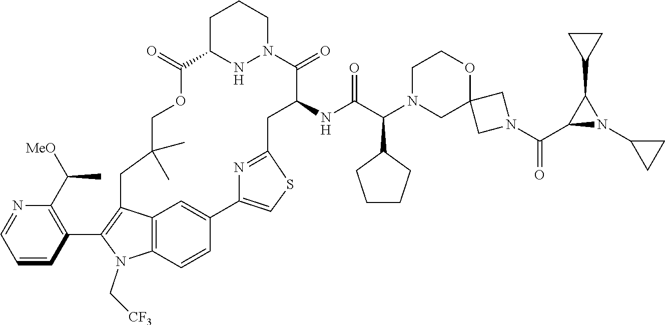 |
| FB 30 | 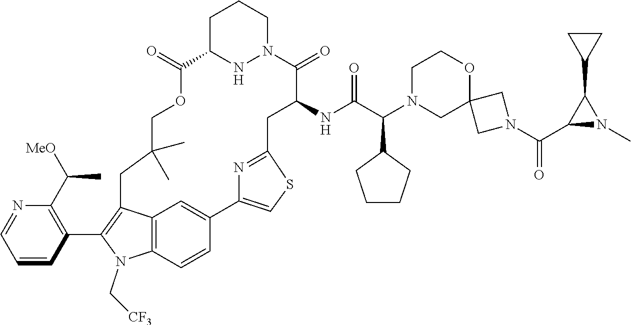 |
| FB 31 | 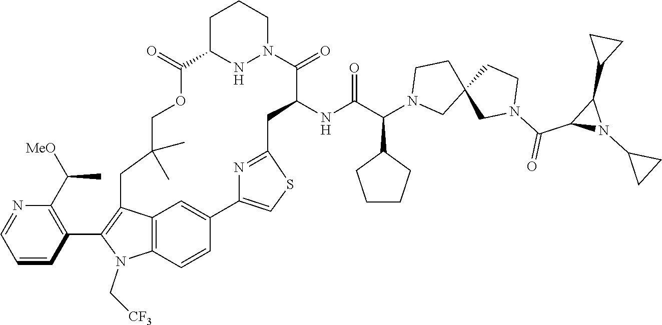 |
| FB 32 | 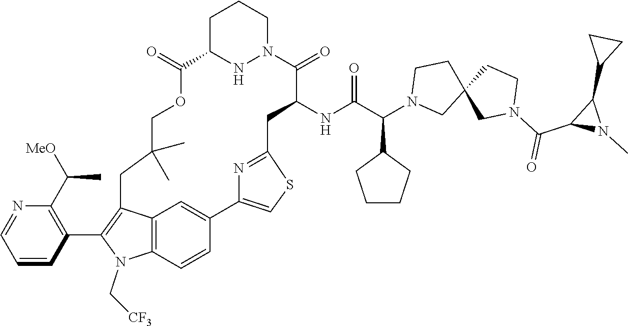 |
| FB 33 | 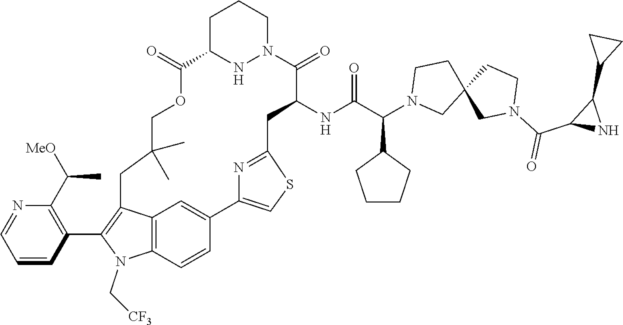 |
| FB 34 | 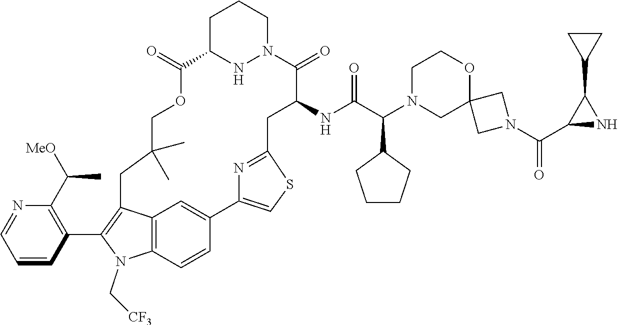 |
| FB 35 | 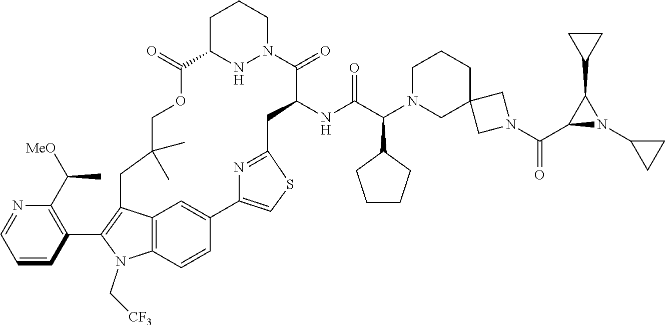 |
| FB 36 | 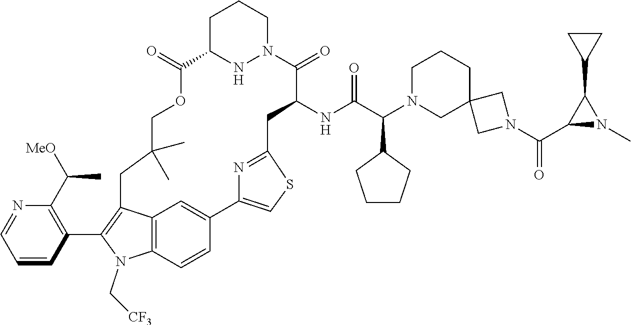 |
| FB 37 | 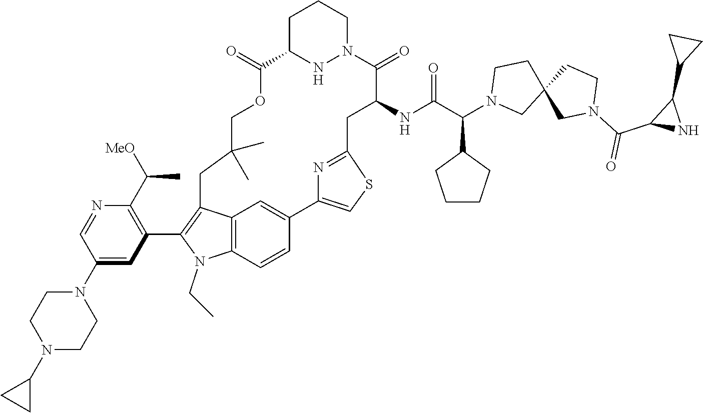 |
| FB 38 | 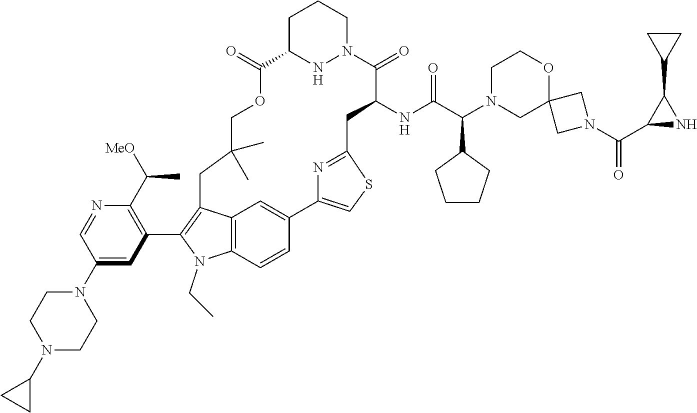 |
| FB 39 | 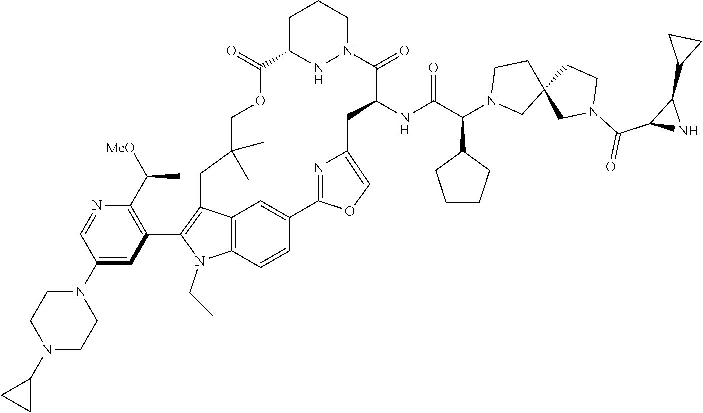 |
| FB 40 | 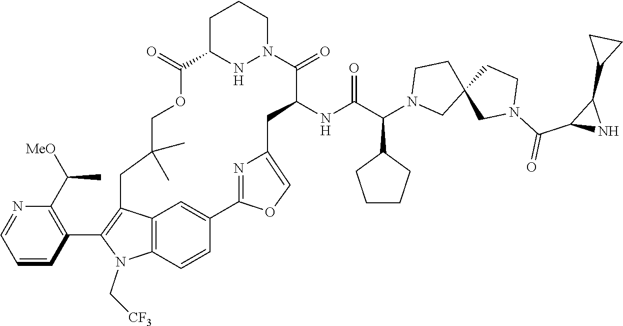 |
| FB 41 | 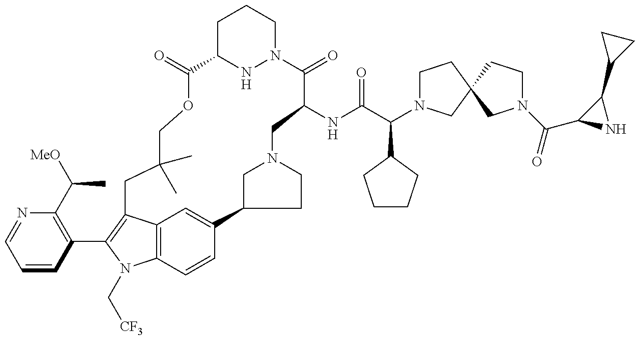 |
| FB 42 | 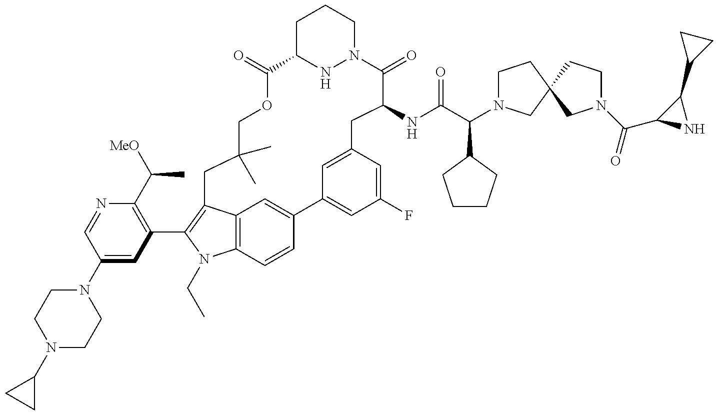 |
| FB 43 | 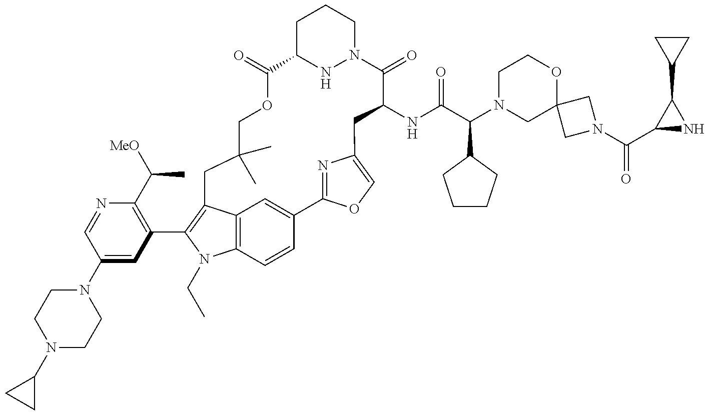 |
| FB 44 | 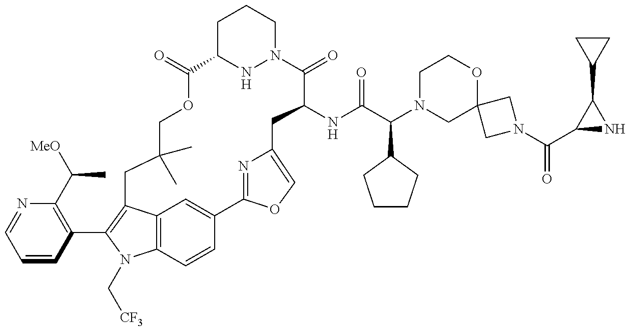 |
| FB 45 | 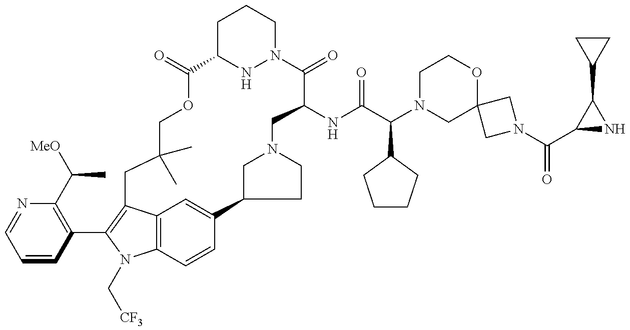 |
| FB 46 | 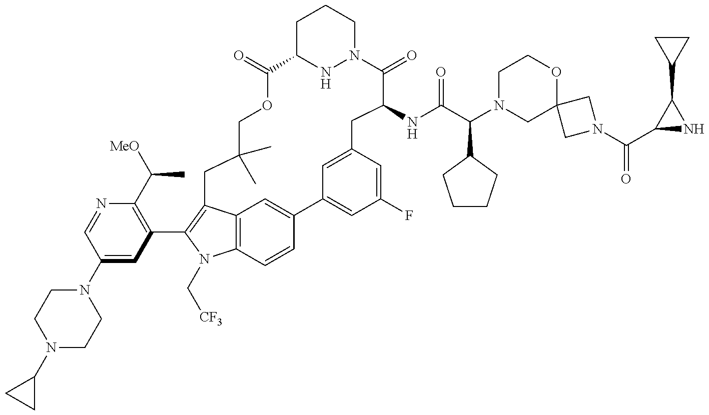 |
| FB 47 | 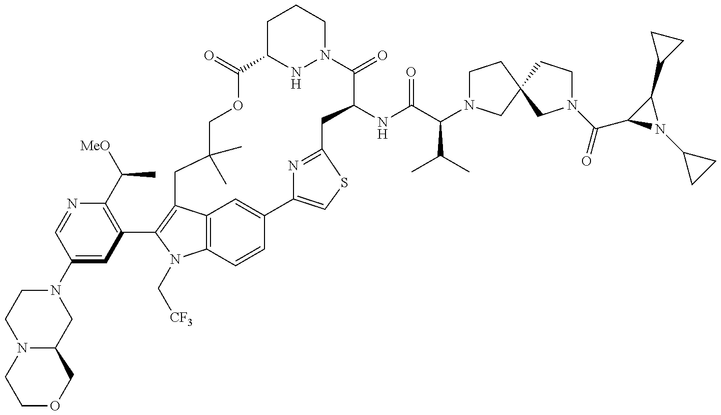 |
| FB 48 | 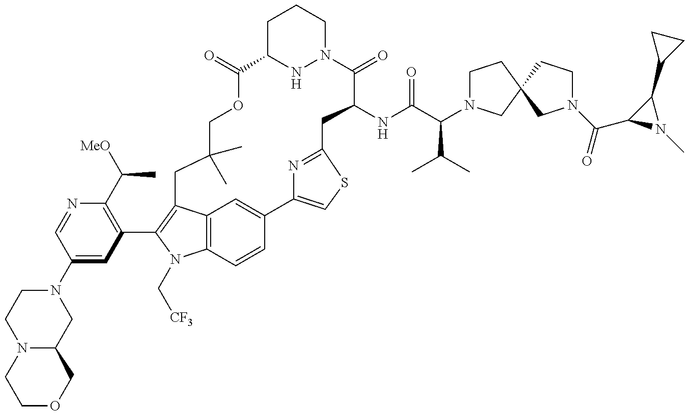 |
| FB 49 | 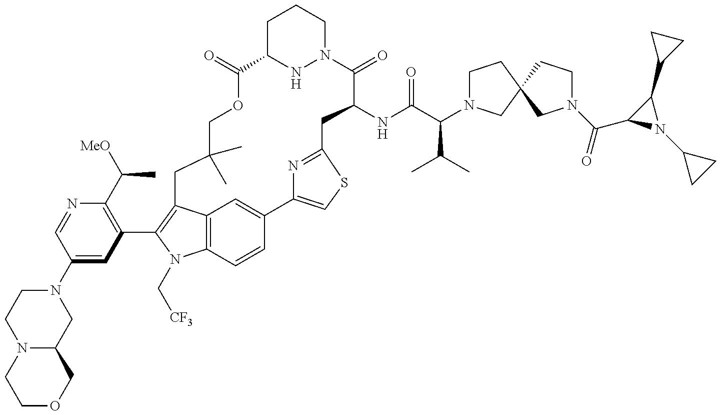 |
| FB 50 | 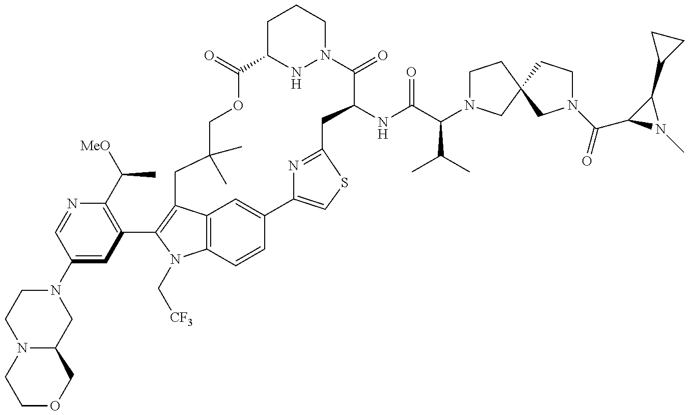 |
| FB 51 | 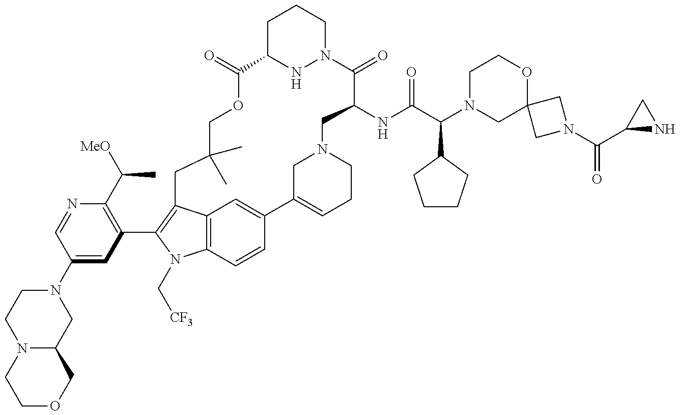 |
| FB 52 | 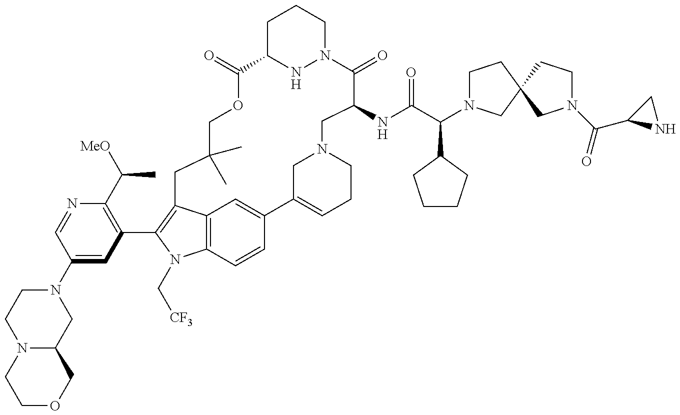 |
| FB 53 | 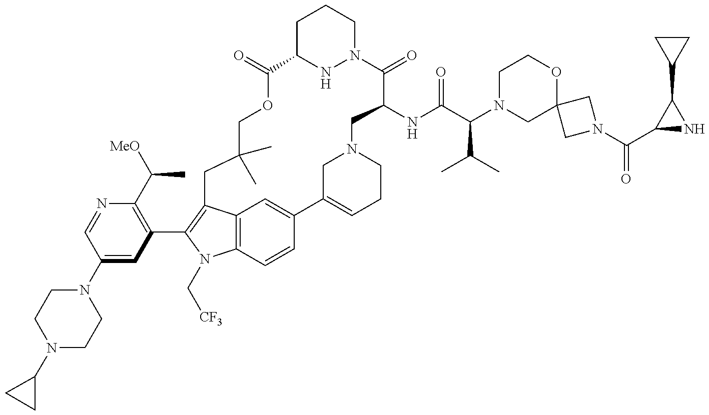 |
| FB 54 | 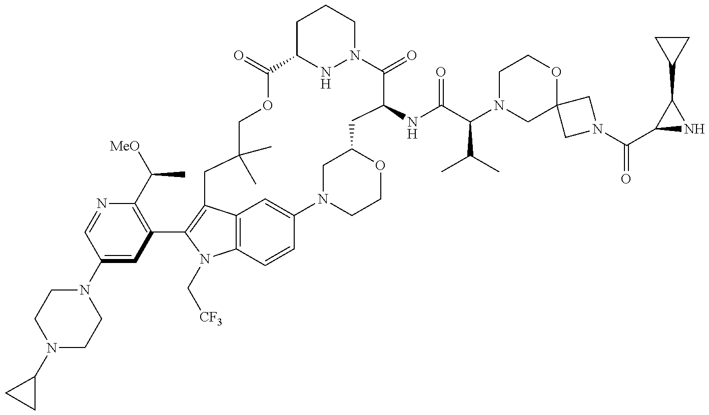 |
| FB 55 | 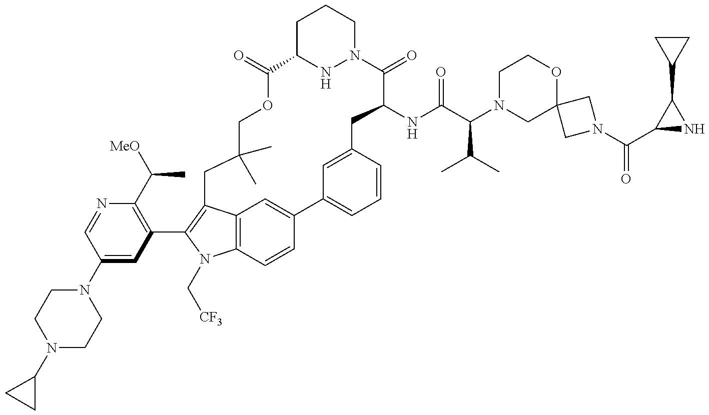 |
| FB 56 | 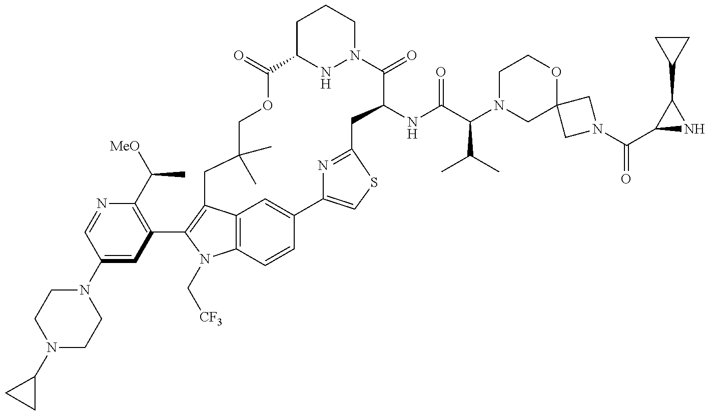 |
| FB 57 | 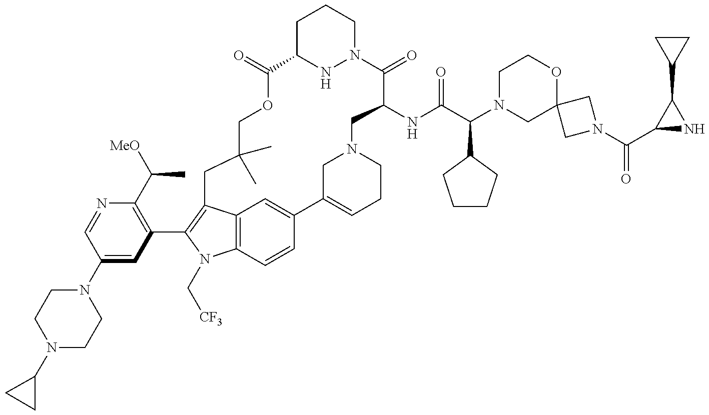 |
| FB 58 | 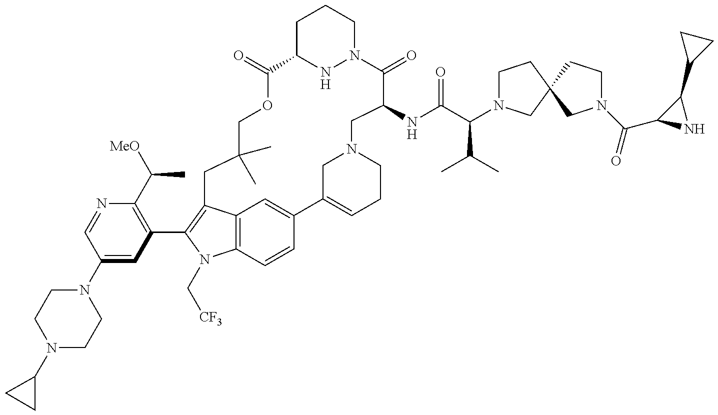 |
| FB 59 | 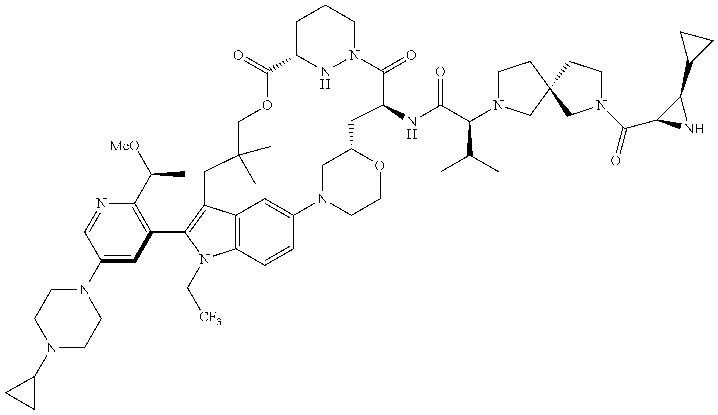 |
| FB 60 | 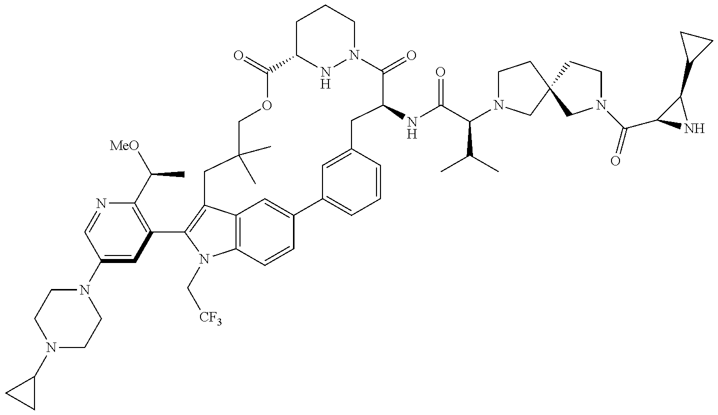 |
| FB 61 | 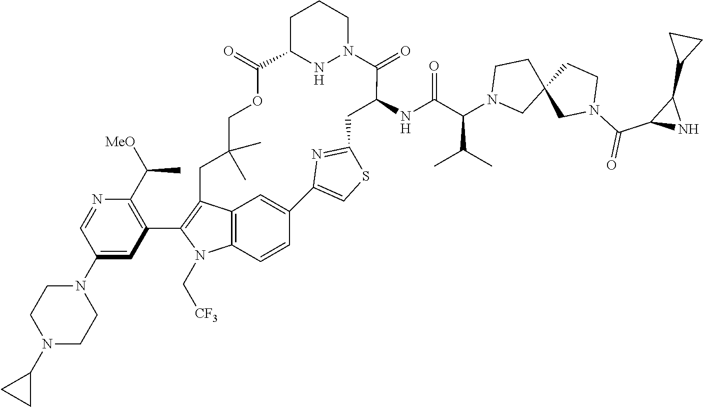 |
| FB 62 | 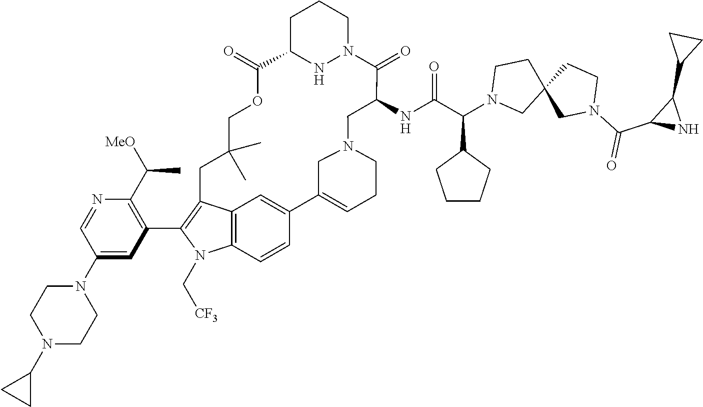 |
| FB 63 | 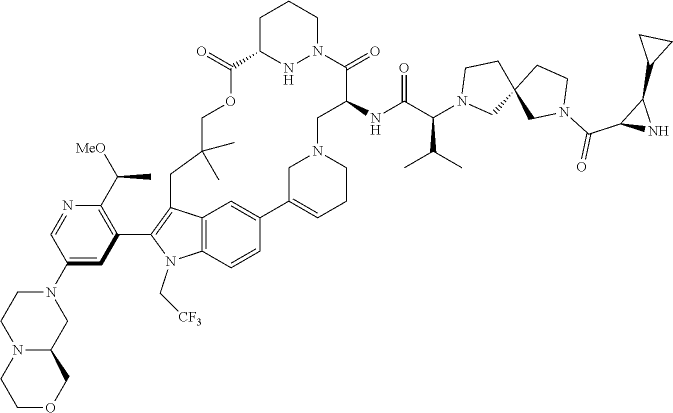 |
| FB 64 | 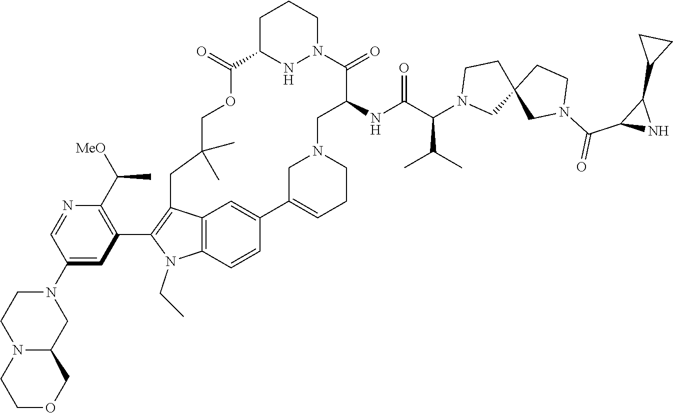 |
| FB 65 | 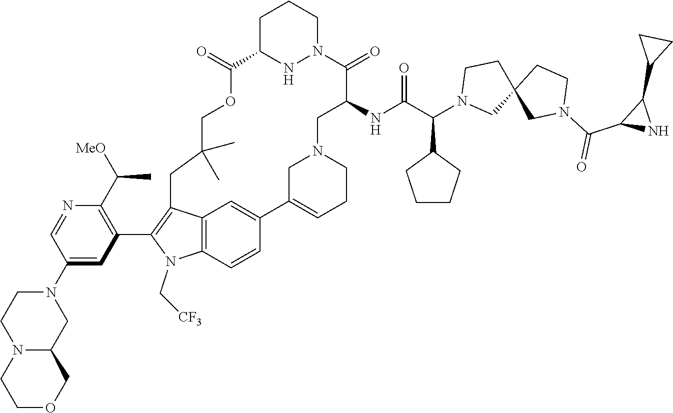 |
| FB 66 | 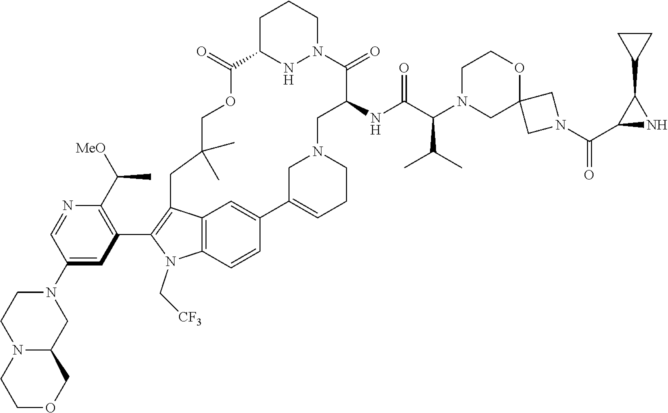 |
| FB 67 | 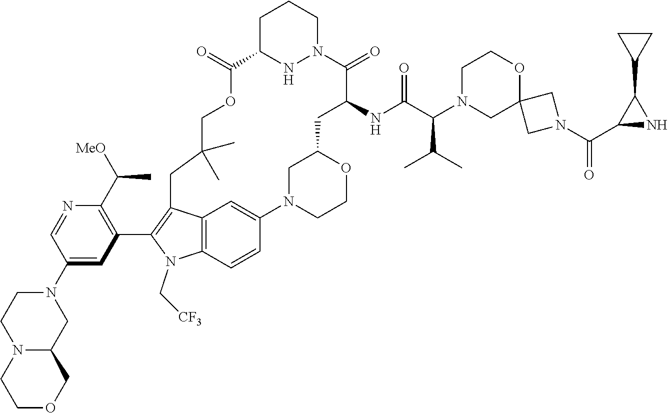 |
| FB 68 | 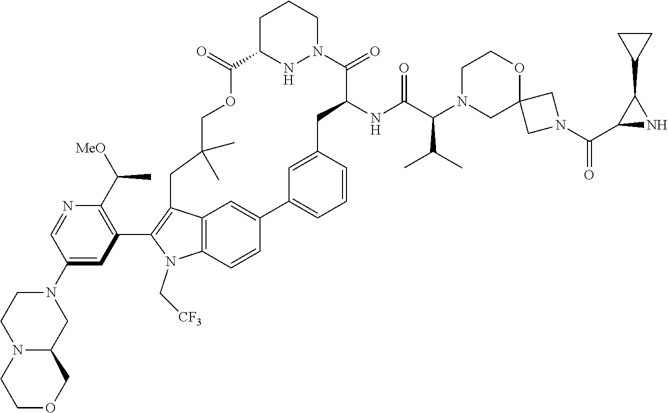 |
| FB 69 | 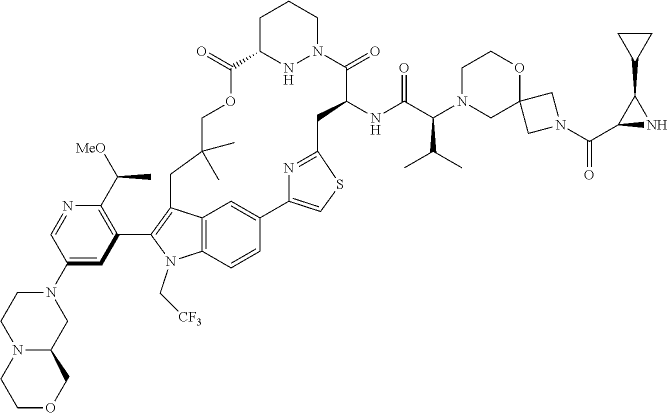 |
| FB 70 | 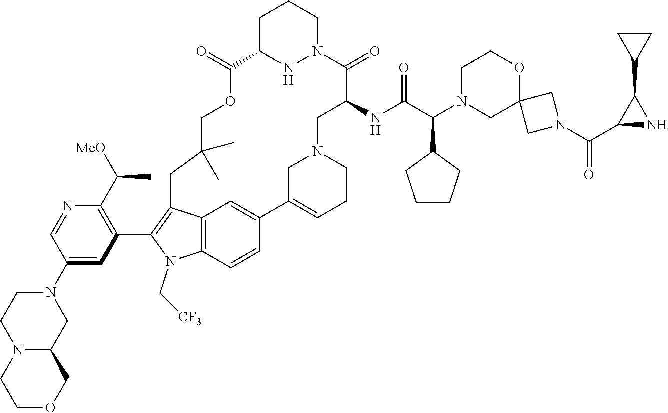 |
| FB 71 | 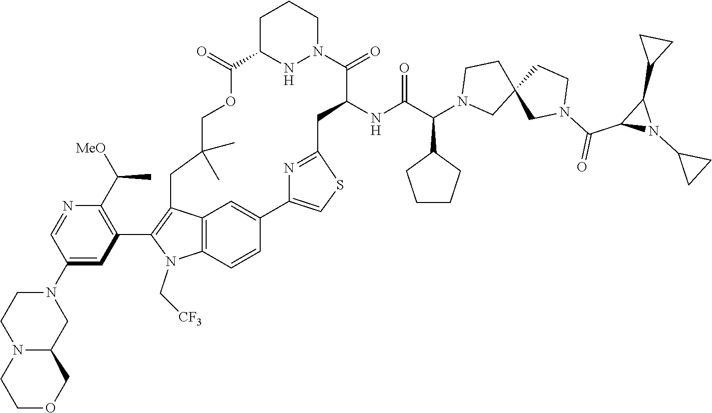 |
| FB 72 | 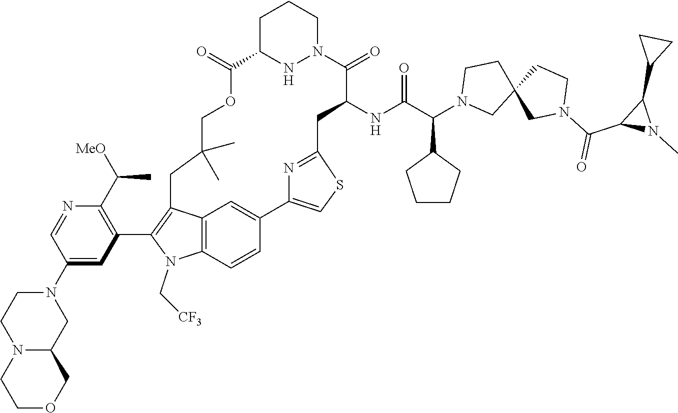 |
| FB 73 | 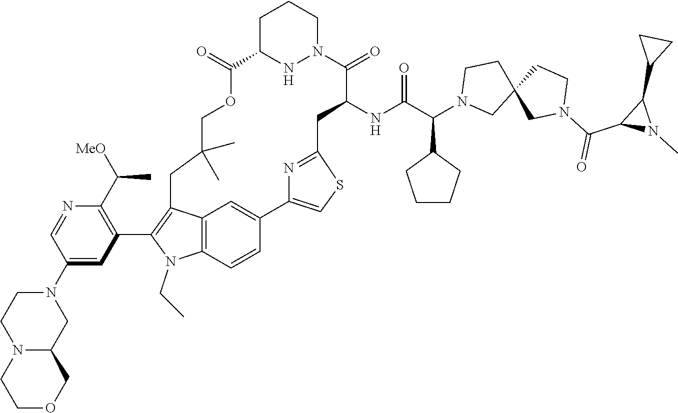 |
| FB 74 | 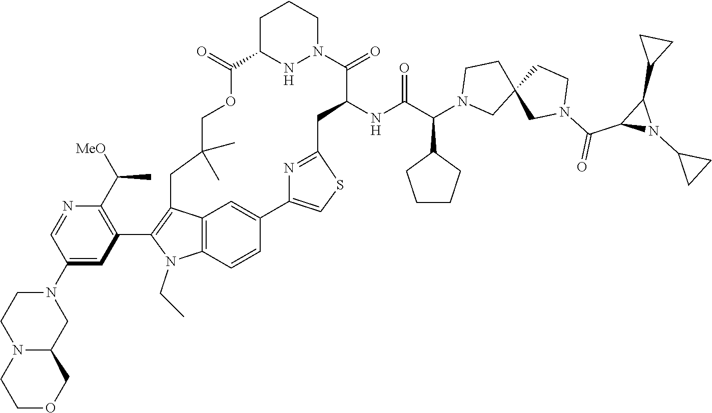 |
| FB 75 | 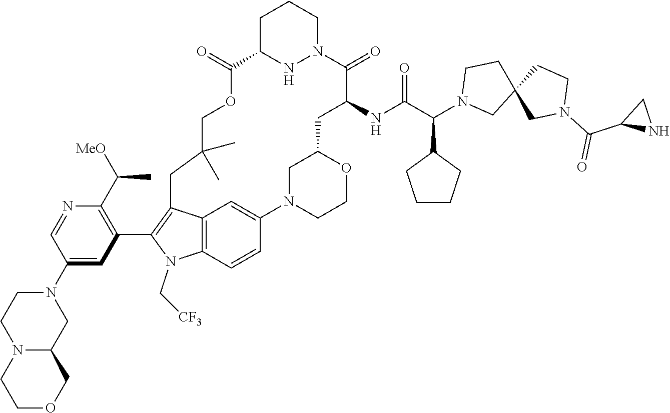 |
| FB 76 | 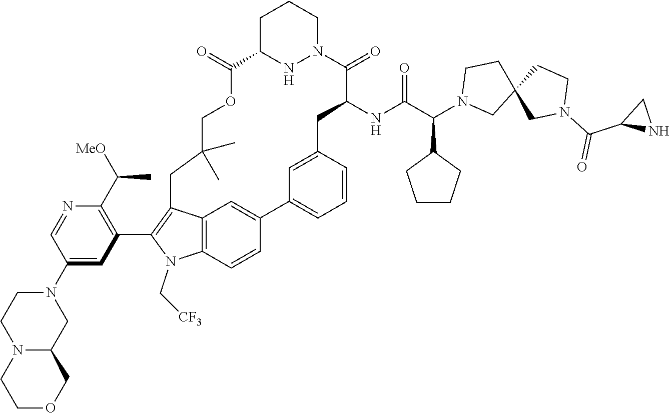 |
| FB 77 | 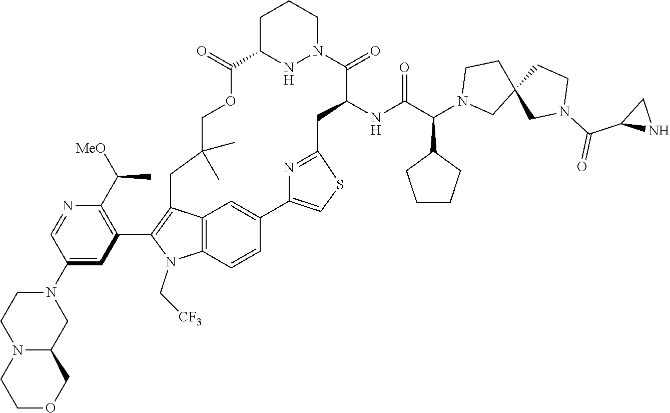 |
| FB 78 | 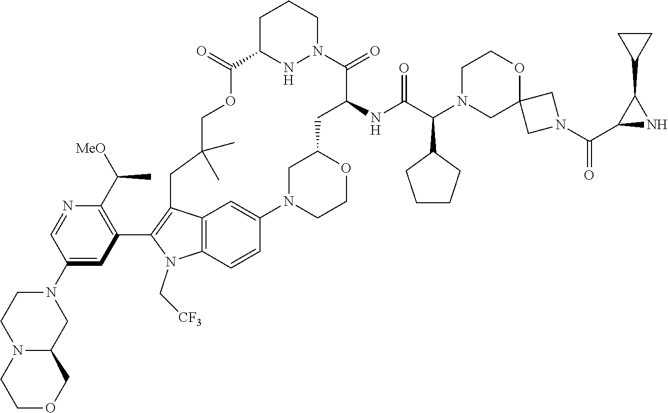 |
| FB 79 | 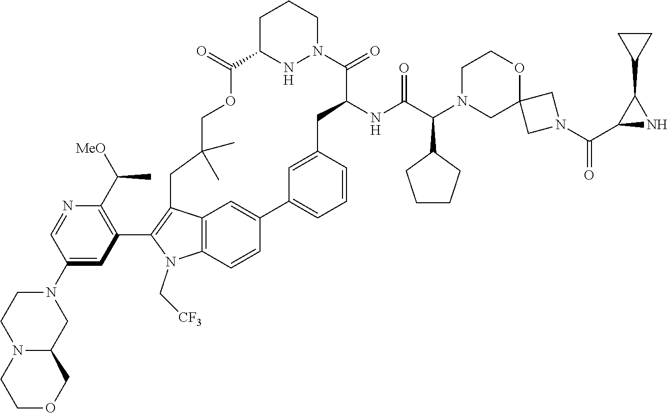 |
| FB 80 | 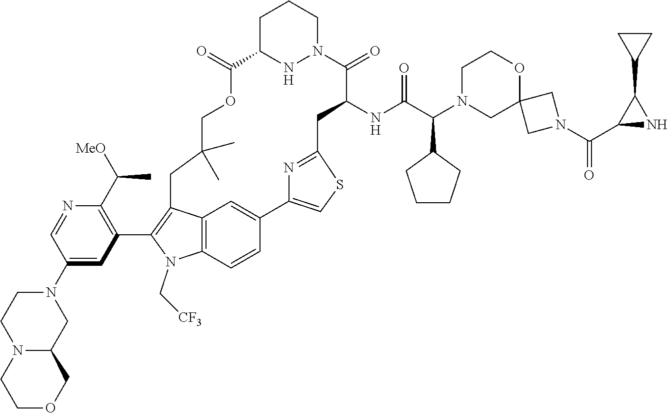 |
| FB 81 | 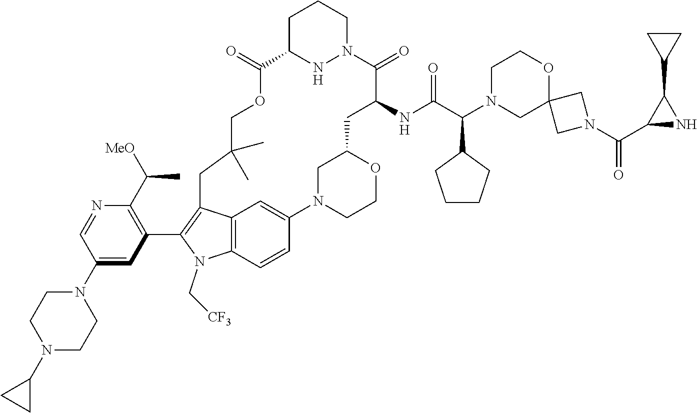 |
| FB 82 | 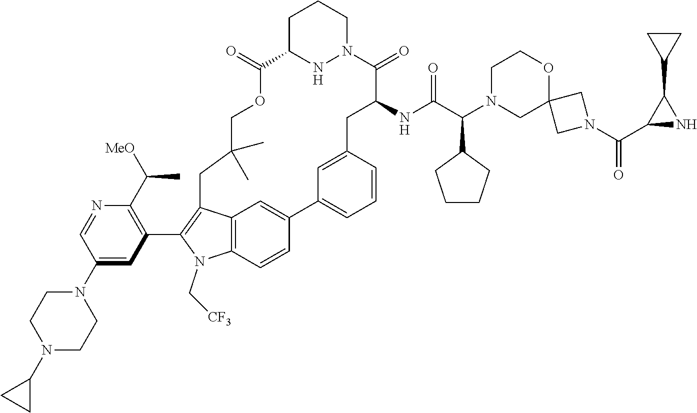 |
| FB 83 | 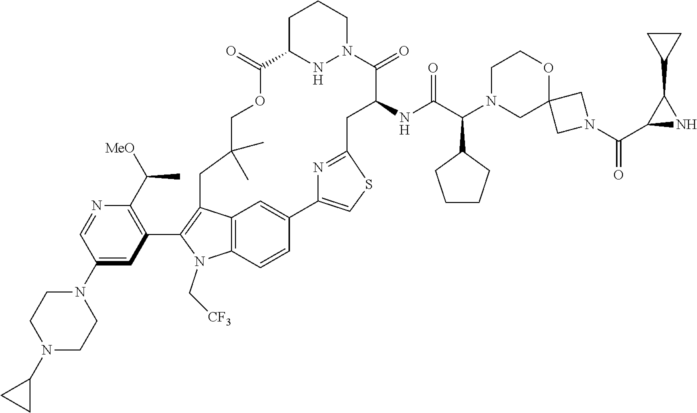 |
| FB 84 | 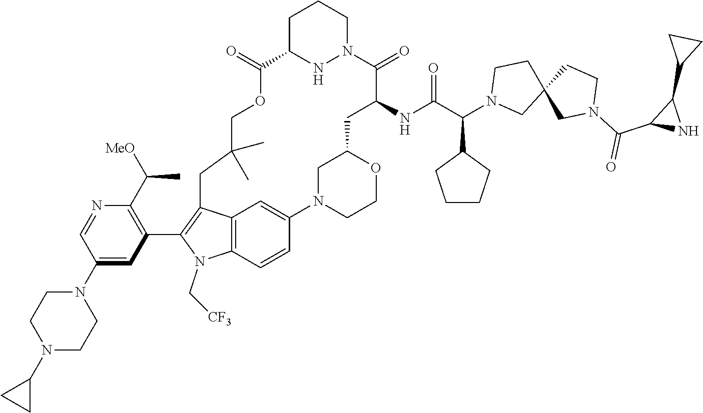 |
| FB 85 | 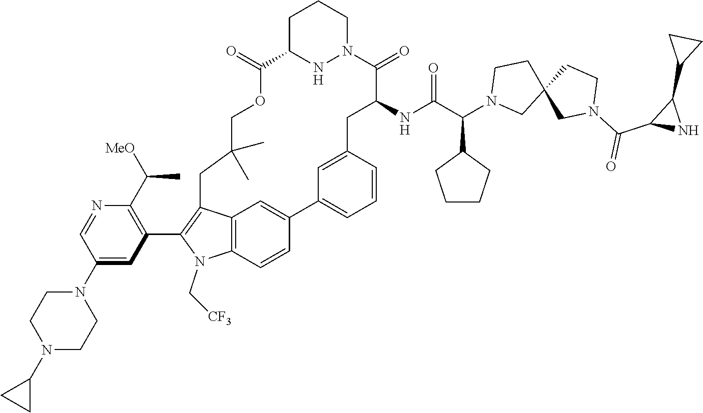 |
| FB 86 | 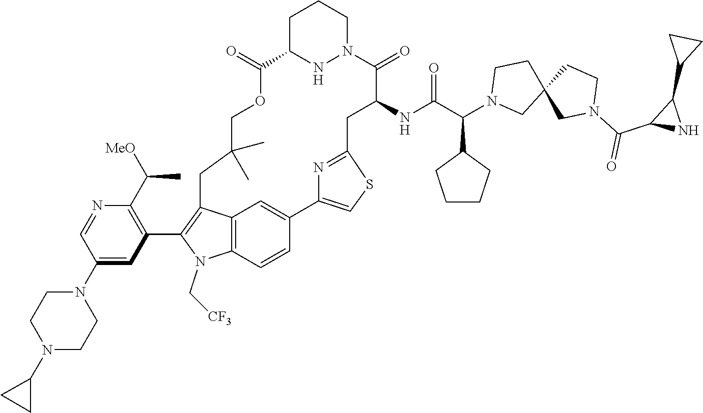 |
| FB 87 | 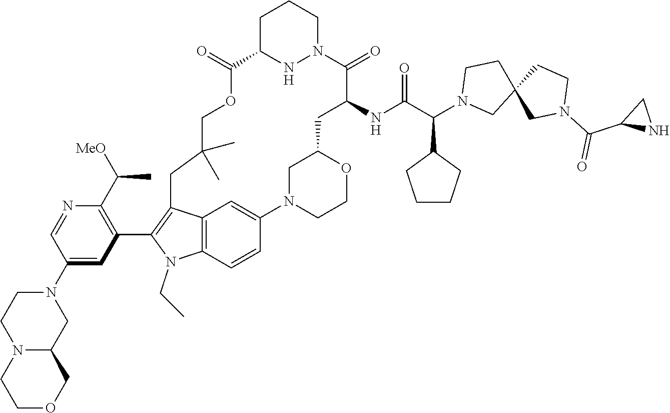 |
| FB 88 | 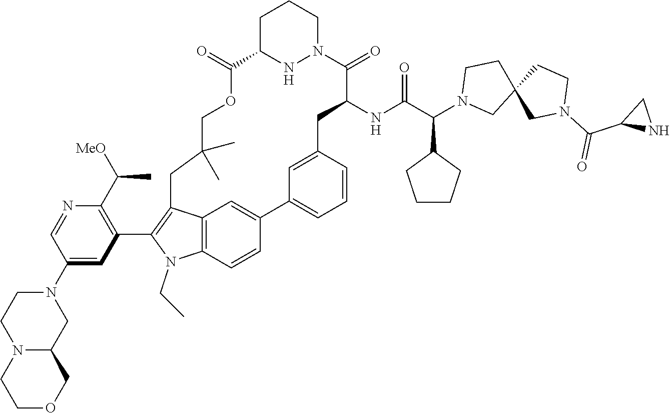 |
| FB 89 | 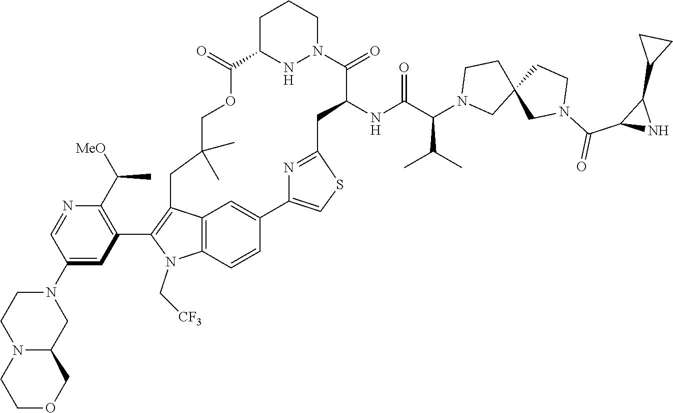 |
| FB 90 | 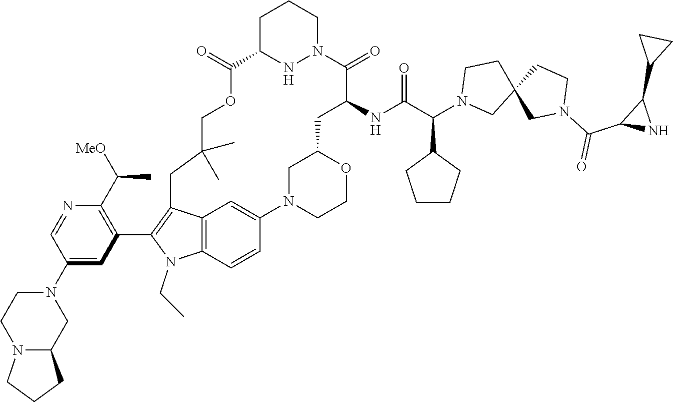 |
| FB 91 | 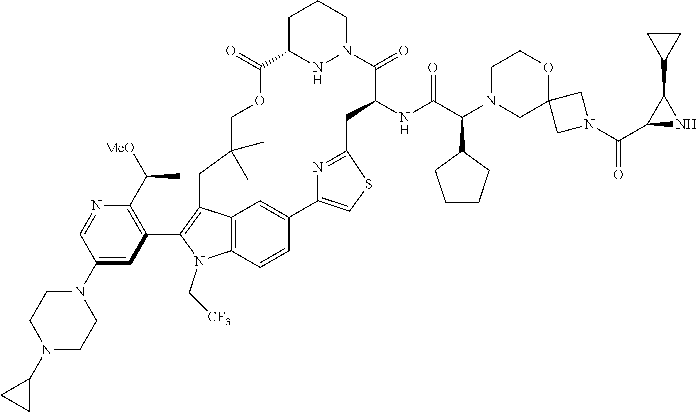 |
| FB 92 | 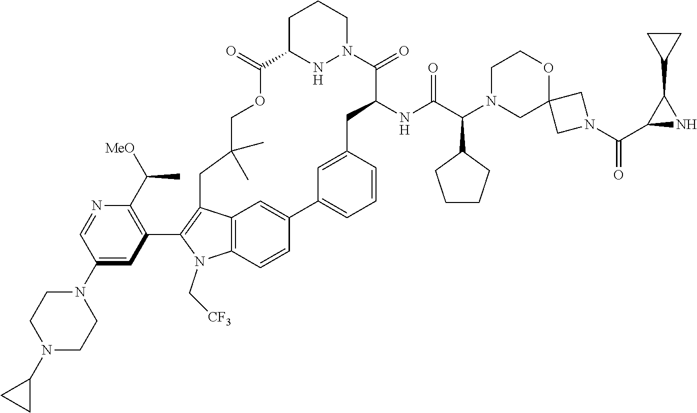 |
| FB 93 | 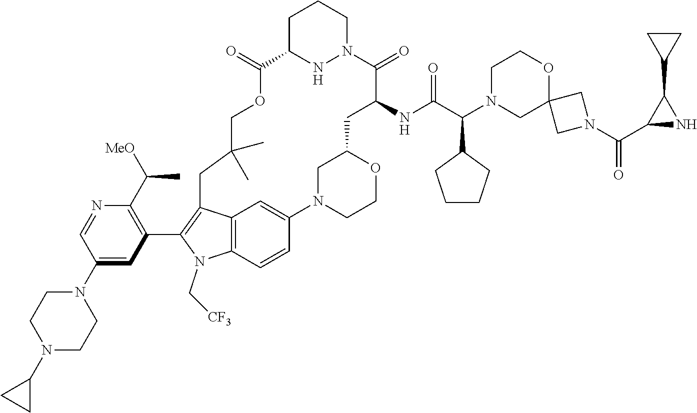 |
| FB 94 | 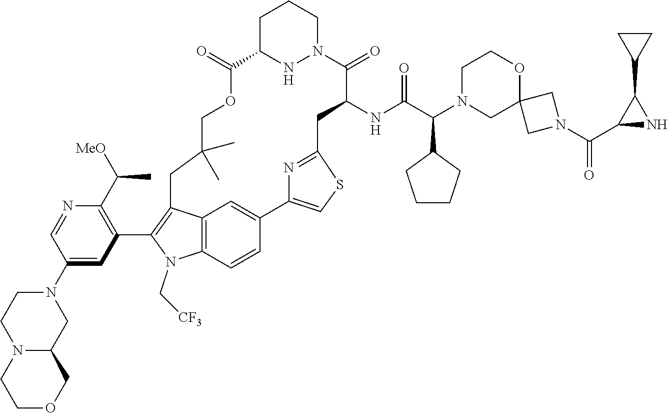 |
| FB 95 | 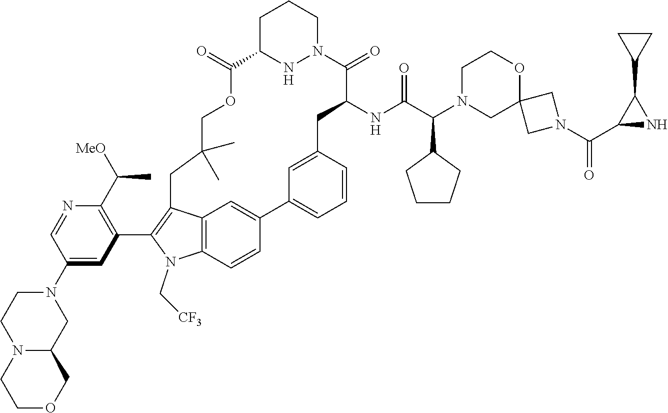 |
| FB 96 | 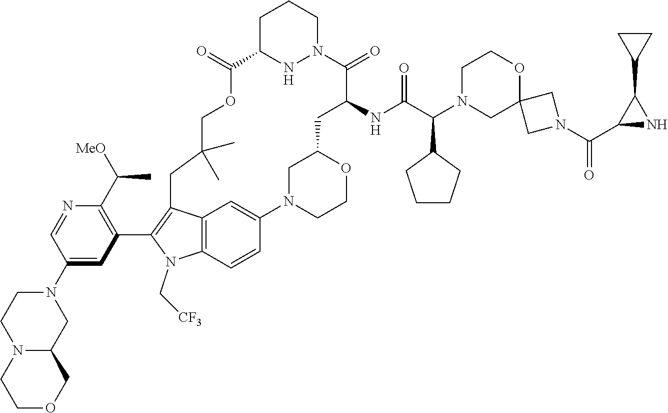 |
| FB 97 | 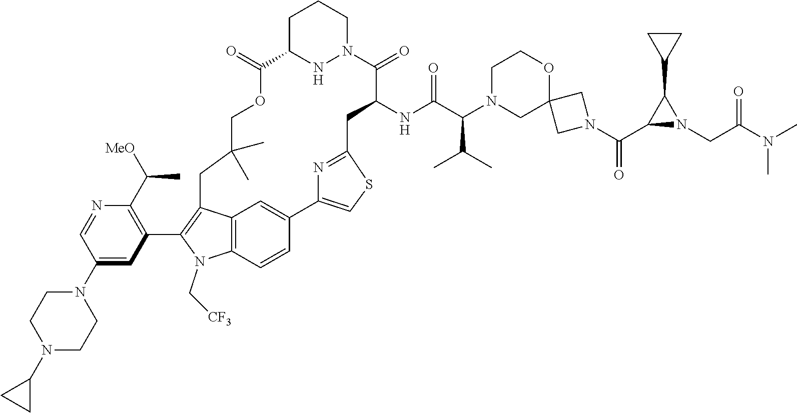 |
| FB 98 | 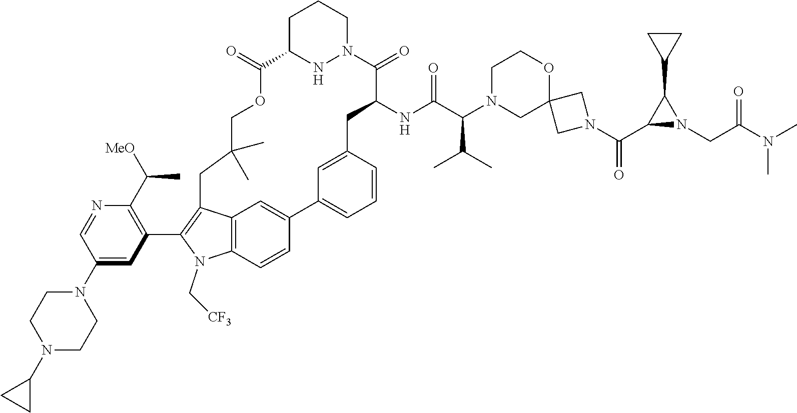 |
| FB 99 | 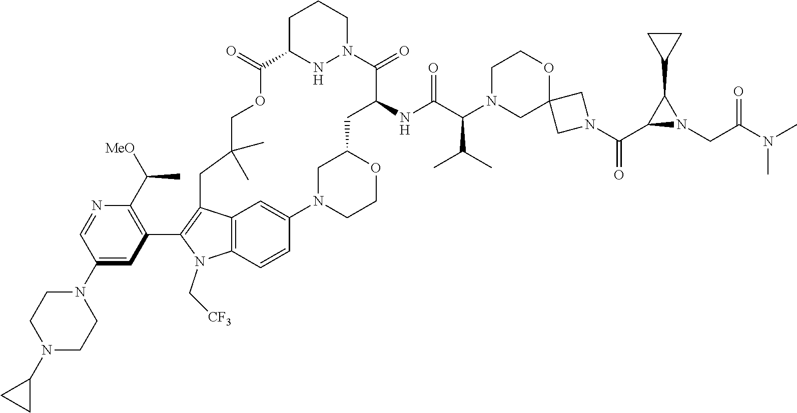 |
| FB 100 | 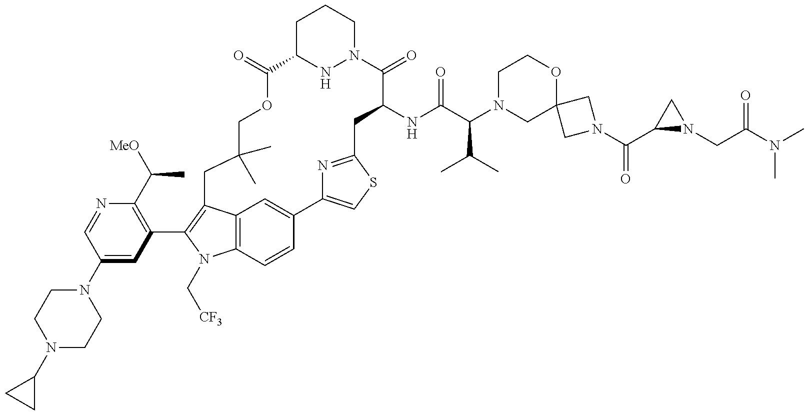 |
| FB 101 | 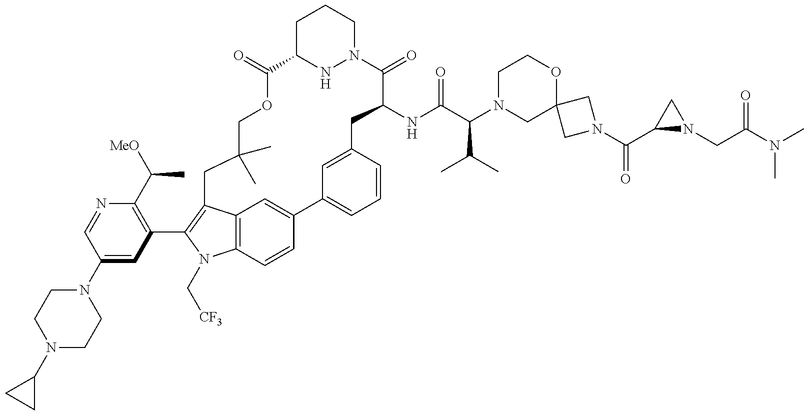 |
| FB 102 | 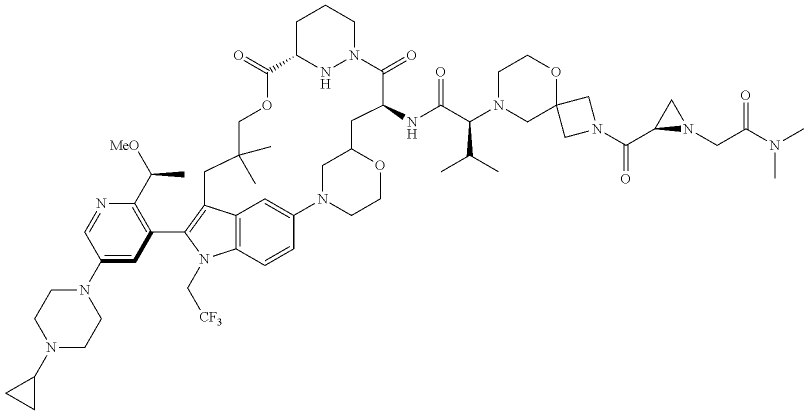 |
| FB 103 | 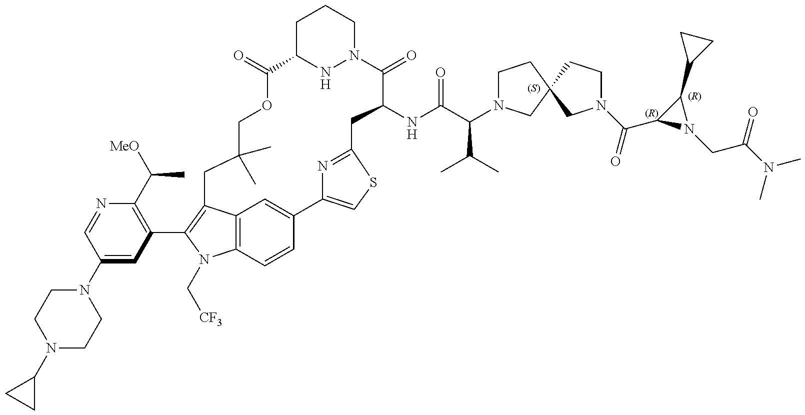 |
| FB 104 | 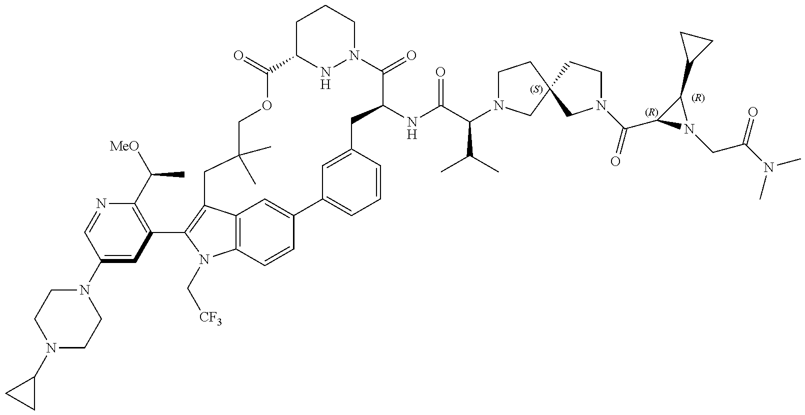 |
| FB 105 | 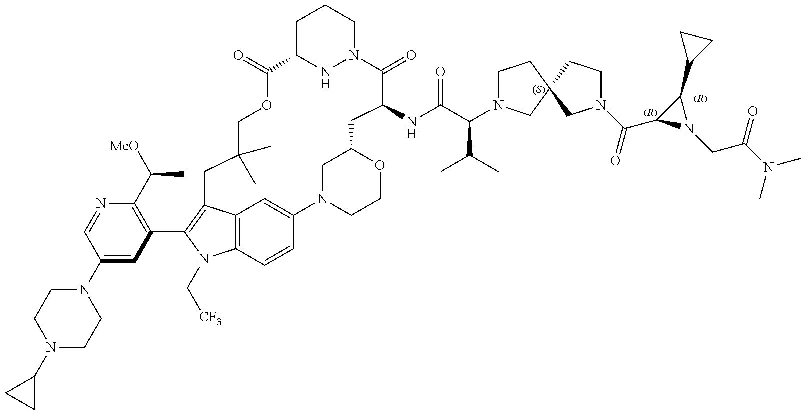 |
| FB 106 | 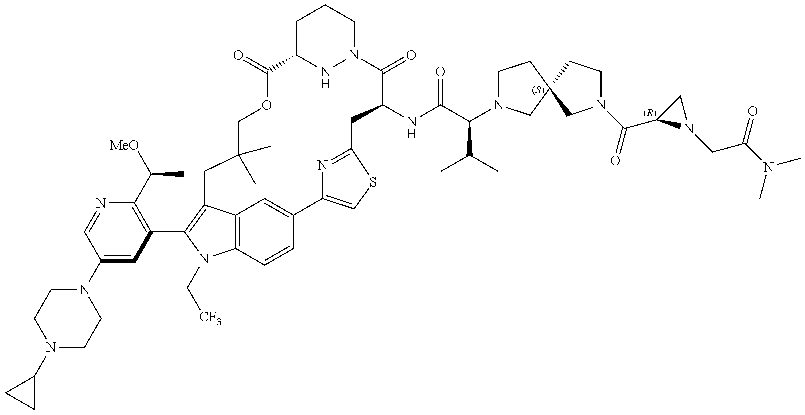 |
| FB 107 | 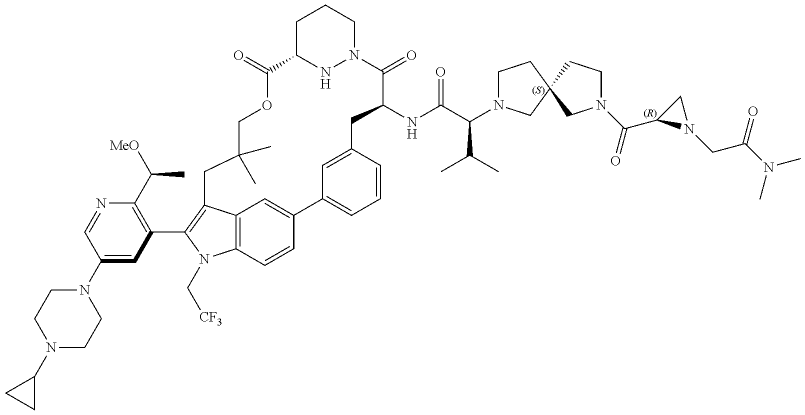 |
| FB 108 | 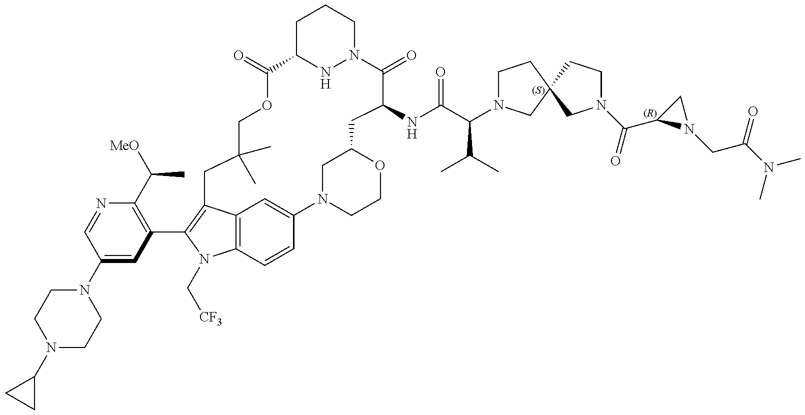 |
| FB 109 | 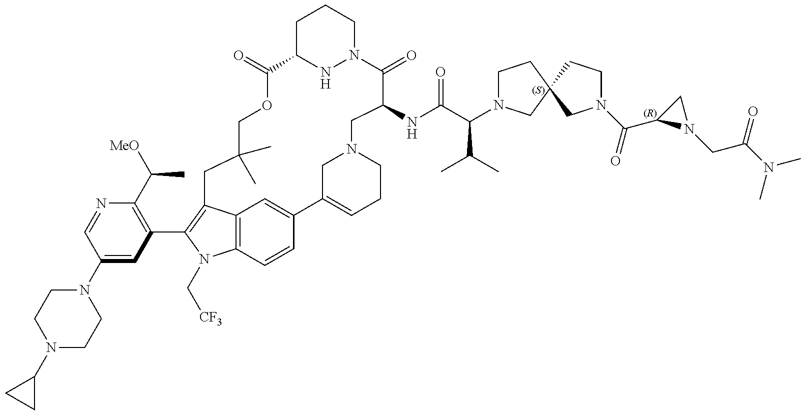 |
| FB 110 | 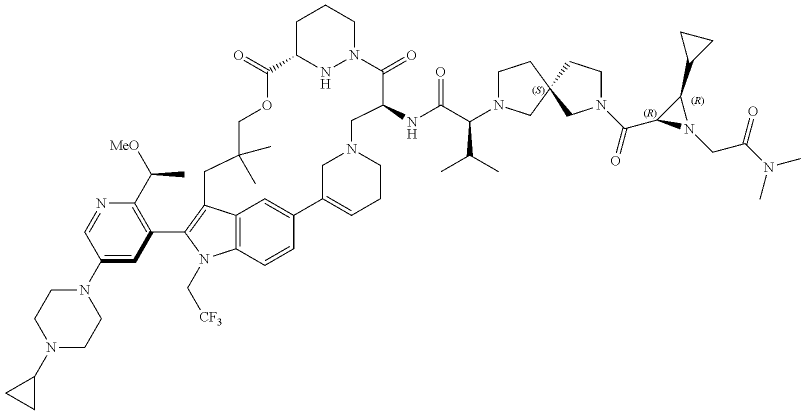 |
| FB 111 | 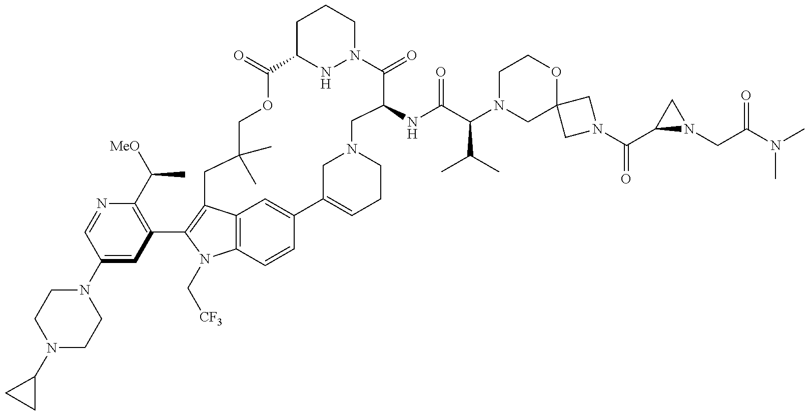 |
| FB 112 | 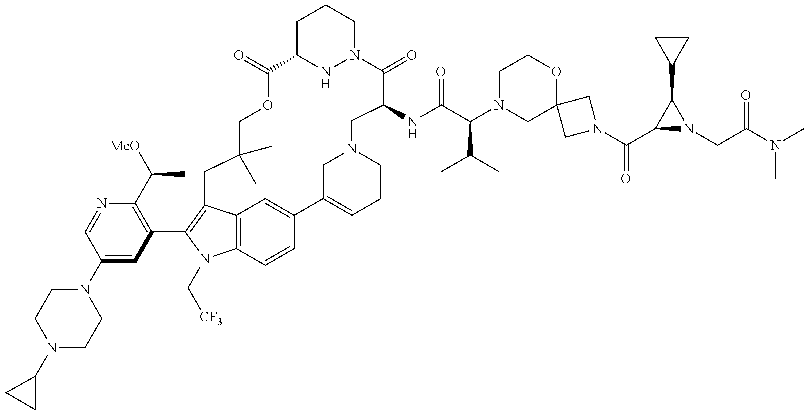 |
| FB 113 | 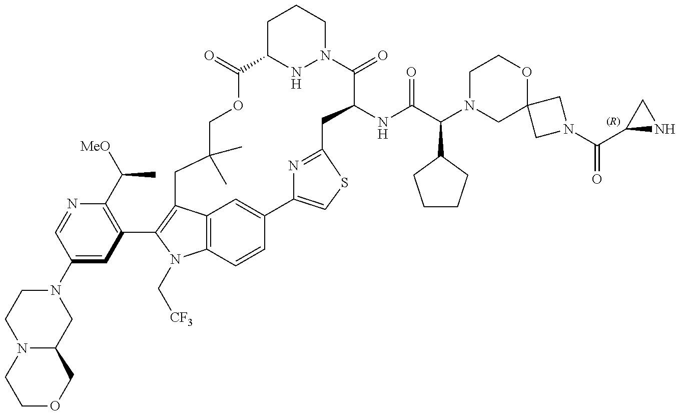 |
| FB 114 | 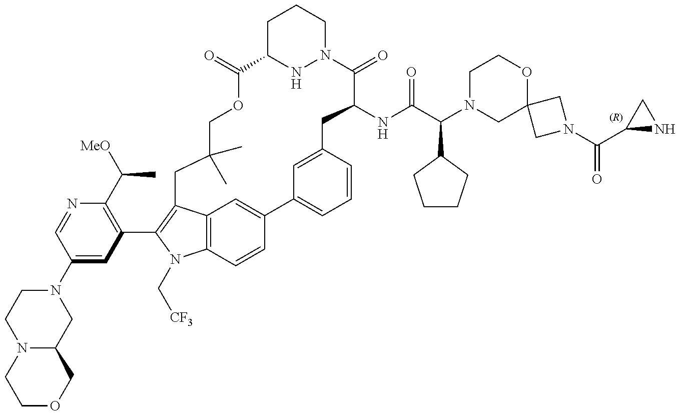 |
| FB 115 | 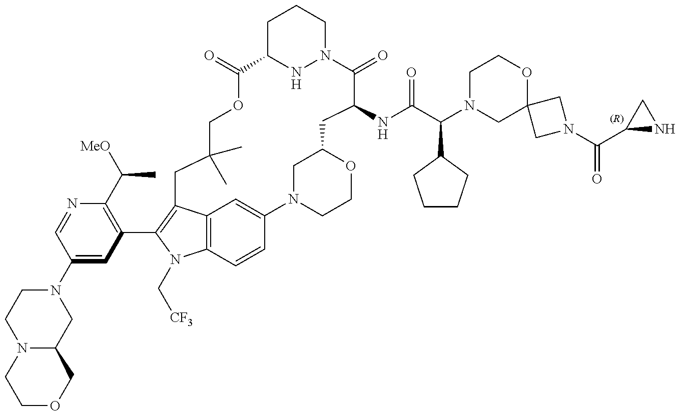 |
| FB 116 | 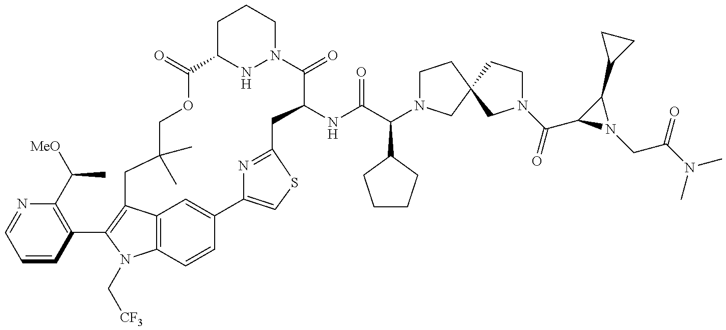 |
| FB 117 | 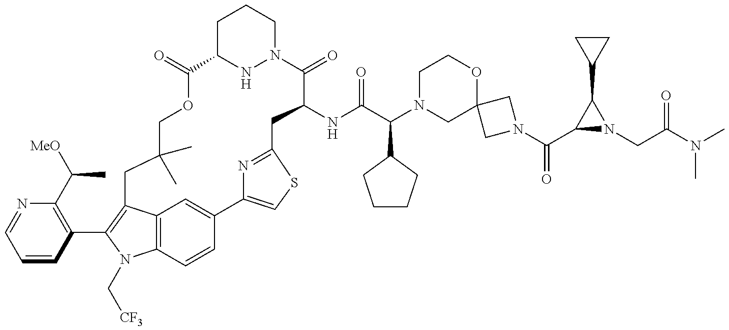 |
| FB 118 | 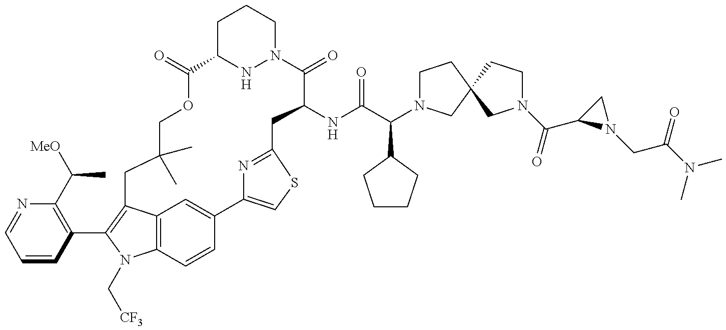 |
| FB 119 | 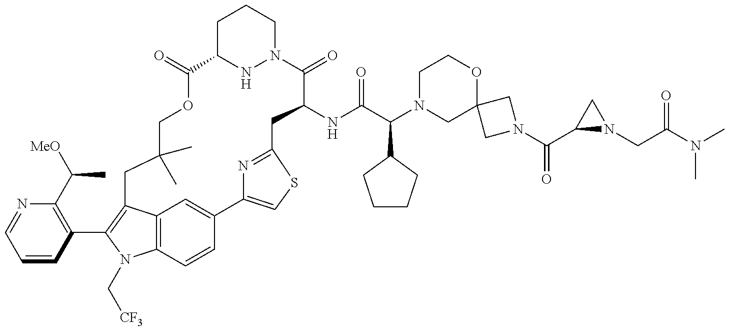 |

Note that some compounds are shown with bonds as flat or wedged. In some instances, the relative stereochemistry of stereoisomers has been determined; in some instances, the absolute stereochemistry has been determined. All stereoisomers of the compounds of the foregoing table are contemplated by the present invention. In particular embodiments, an atropisomer of a compound of the foregoing table is contemplated.

In some embodiments, the RAS(ON) inhibitor is a compound of Formula FIII, or pharmaceutically acceptable salt thereof:

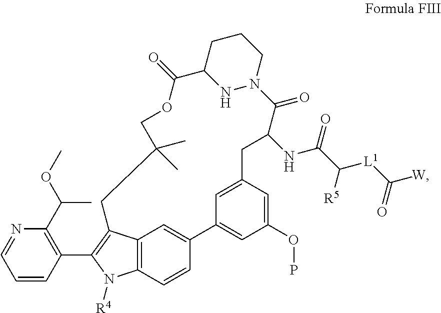

wherein P is —(CO)R⁹, —(PO)(OH)₂, or —Si(R¹⁰)₃;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
L¹ is optionally substituted 3 to 9-membered heterocycloalkylene or optionally substituted C₂-C₄ heteroalkylene;
R⁴ is optionally substituted C₁-C₆ alkyl;
R⁵ is optionally substituted C₁-C₆ alkyl or optionally substituted 3 to 6-membered cycloalkyl;
R⁹ is optionally substituted C₁-C₃ alkyl or optionally substituted C₁-C₃ heteroalkyl; and each R¹⁰ is, independently, optionally substituted C₁-C₃ alkyl.

In some embodiments, W is a cross-linking group comprising an aziridine.

In some embodiments of Formula FIII or subformula thereof, R⁴ is:

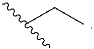 [SMILES: *CC]

In some embodiments of Formula FIII or subformula thereof, R⁵ is optionally substituted C₁-C₆ alkyl. In some embodiments, R⁵ is:

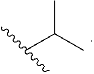 [SMILES: *C(C)C]

In some embodiments of Formula FIII or subformula thereof, R⁵ is optionally substituted 3 to 6-membered cycloalkyl. In some embodiments, R⁵ is:

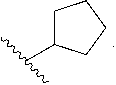 [SMILES: *C1CCCC1]

In some embodiments of Formula FIII or subformula thereof, P is —(CO)R⁹. In some embodiments, P is selected from:

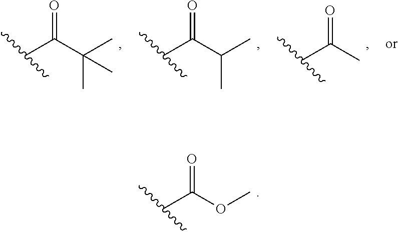

In some embodiments of Formula FIII or subformula thereof, P is —(PO)(OH)₂.

In some embodiments of Formula FIII or subformula thereof, P is —Si(R¹⁰)₃. In some embodiments, P is selected from:

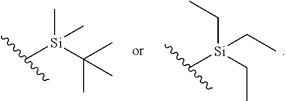

In some embodiments of Formula FIII or subformula thereof, L¹ is 3 to 9-membered heterocycloalkylene. In some embodiments, L¹ is, or a stereoisomer thereof:

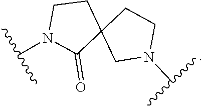 [SMILES: *N1CCC2(CCN(*)C2=O)C1]

In some embodiments of Formula FIII or subformula thereof, L¹ is optionally substituted C₂-C₄ heteroalkylene. In some embodiments, L¹ is:

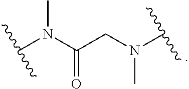 [SMILES: *N(C)CC(=O)N(*)C]

In some embodiments of Formula FI, Formula FIII or subformula thereof, W is an optionally substituted cyclopropyl-aziridinyl moiety. In some embodiments, W is, or a stereoisomer thereof:

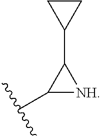 [SMILES: *C1NC1C1CC1]

In some embodiments, W is, or a stereoisomer thereof:

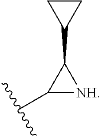 [SMILES: *C1N[C@@H]1C1CC1]

In some embodiments, W is:

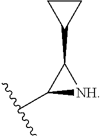 [SMILES: *[C@@H]1N[C@@H]1C1CC1]

In some embodiments, W is:

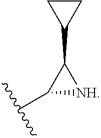 [SMILES: *[C@H]1N[C@@H]1C1CC1]

In some embodiments, W is, or a stereoisomer thereof:

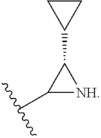 [SMILES: *C1N[C@H]1C1CC1]

In some embodiments, W is, or a stereoisomer thereof:

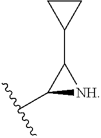

In some embodiments, W is, or a stereoisomer thereof:

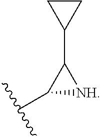

In some embodiments, W is:

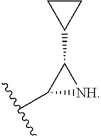 [SMILES: *[C@H]1N[C@H]1C1CC1]

In some embodiments, W is:

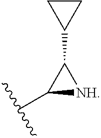 [SMILES: *[C@@H]1N[C@H]1C1CC1]

In some embodiments, W is, or a stereoisomer thereof:

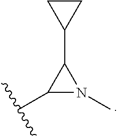 [SMILES: *C1C(C2CC2)N1C]

In some embodiments, W is, or a stereoisomer thereof:

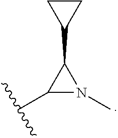 [SMILES: *C1[C@@H](C2CC2)N1C]

In some embodiments, W is:

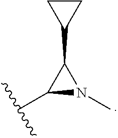

In some embodiments, W is:

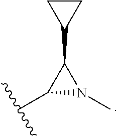

In some embodiments, W is, or a stereoisomer thereof:

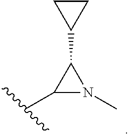 [SMILES: *C1[C@H](C2CC2)N1C]

In some embodiments, W is, or a stereoisomer thereof:

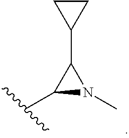

In some embodiments, W is, or a stereoisomer thereof:

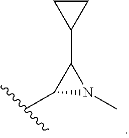

In some embodiments, W is:

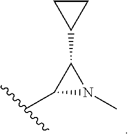

In some embodiments, W is:

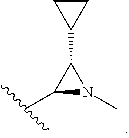

In some embodiments, the RAS(ON) inhibitor is selected from Table F3, or a pharmaceutically acceptable salt or stereoisomer thereof. In some embodiments, the RAS(ON) inhibitor is selected from Table F3, or a pharmaceutically acceptable salt or atropisomer thereof.

TABLE F3
Certain Compounds of the Present Invention

| Ex # | Structure |
|---|---|
| FC 1 | 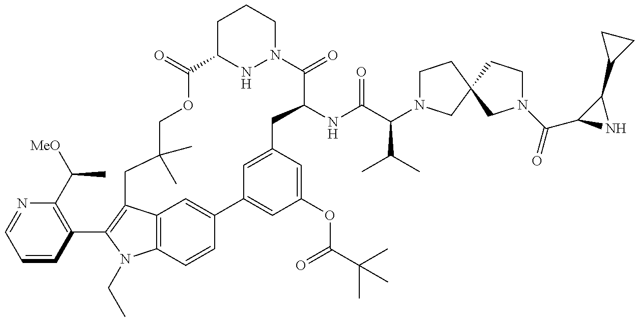 |
| FC 2 | 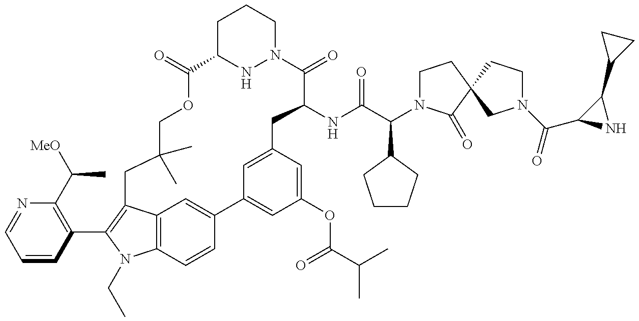 |
| FC 3 | 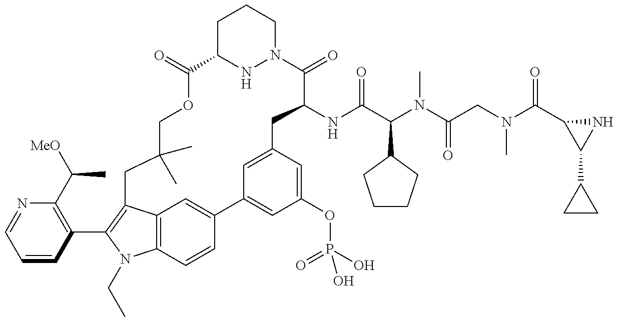 |
| FC 4 | 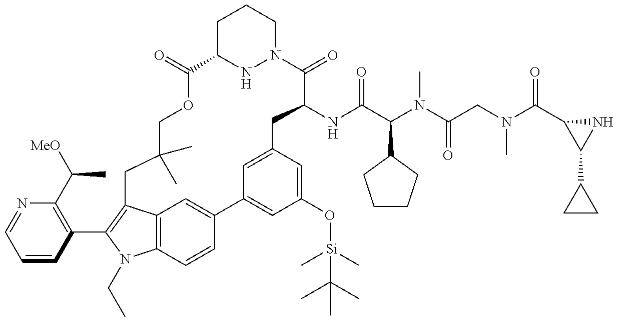 |
| FC 5 | 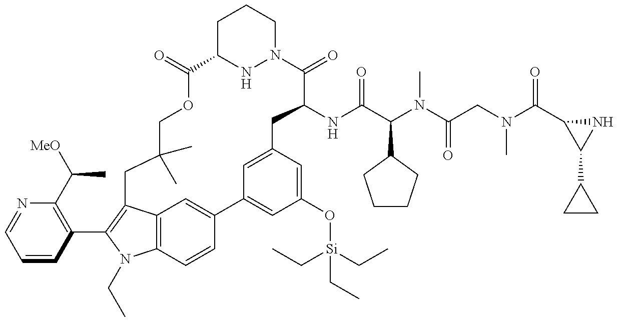 |
| FC 6 | 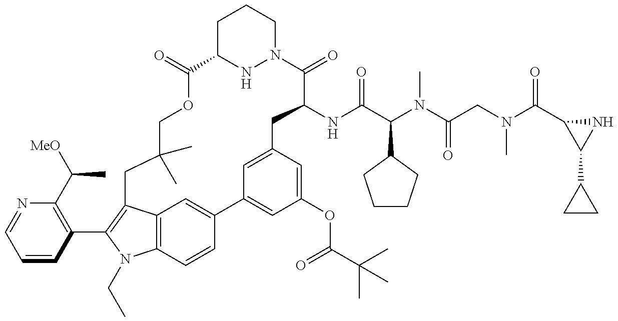 |
| FC 7 | 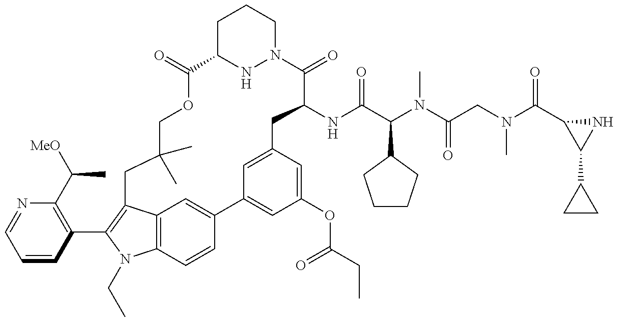 |
| FC 8 | 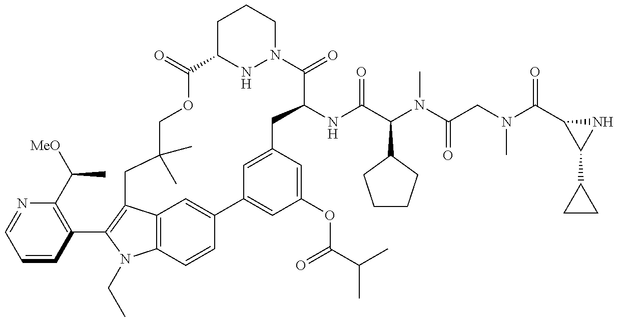 |
| FC 9 | 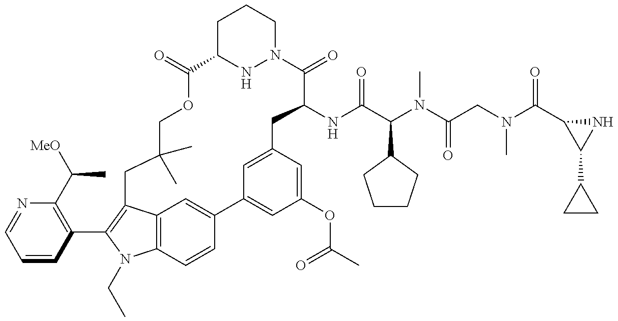 |

In some embodiments, the RAS(ON) inhibitor is a compound selected from Table F4, or a pharmaceutically acceptable salt or stereoisomer thereof. In some embodiments, the RAS(ON) inhibitor is selected from Table F4, or a pharmaceutically acceptable salt or atropisomer thereof.

TABLE F4
Certain Compounds of the Present Invention

| Ex # | Structure |
|---|---|
| FD 1 | 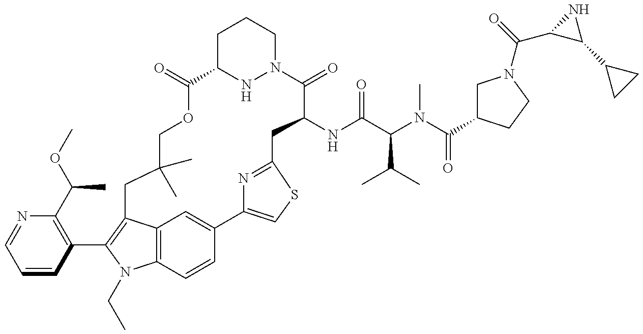 |
| FD 2 | 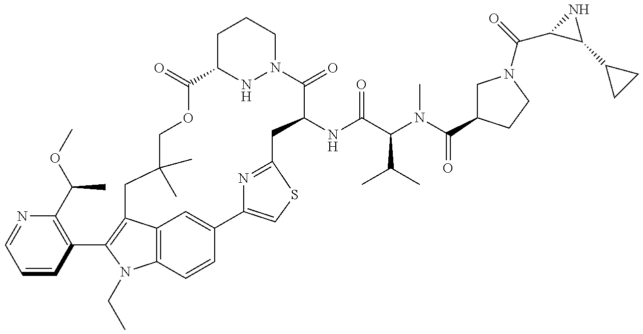 |
| FD 3 | 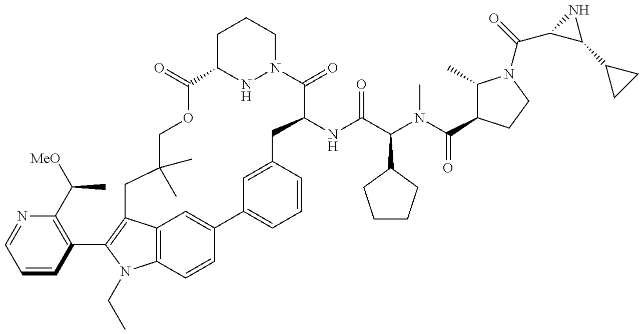 |
| FD 4 | 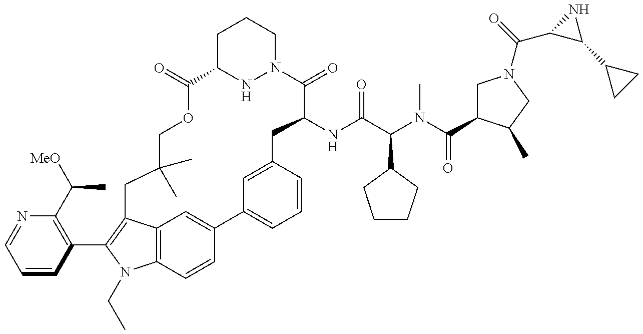 |
| FD 5 | 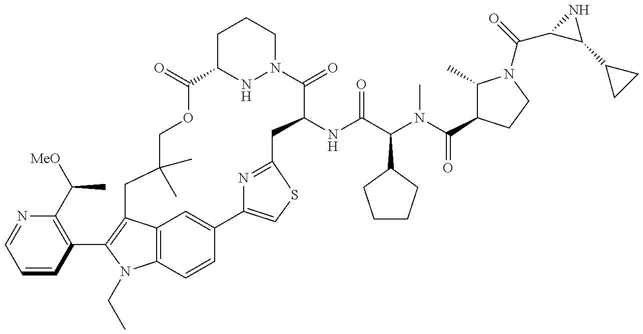 |
| FD 6 | 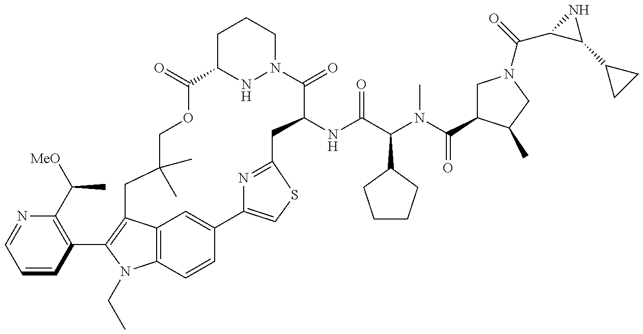 |

In some embodiments, the RAS(ON) inhibitor is a compound of Table E5, or a pharmaceutically acceptable salt thereof. In some embodiments, the RAS(ON) inhibitor is a compound selected from Table F5, or a pharmaceutically acceptable salt or atropisomer thereof.

TABLE F5
Certain Compounds of the Present Invention

| Ex # | Structure |
|---|---|
| FE 1 | 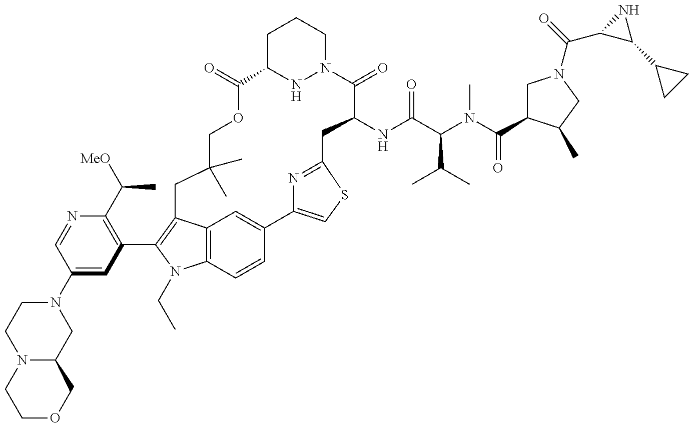 |
| FE 2 | 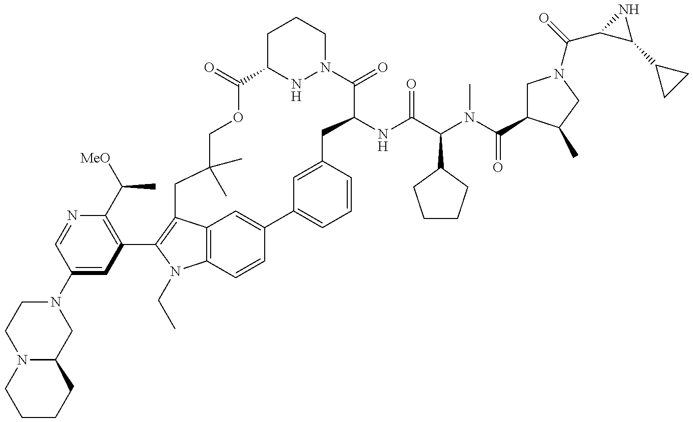 |
| FE 3 | 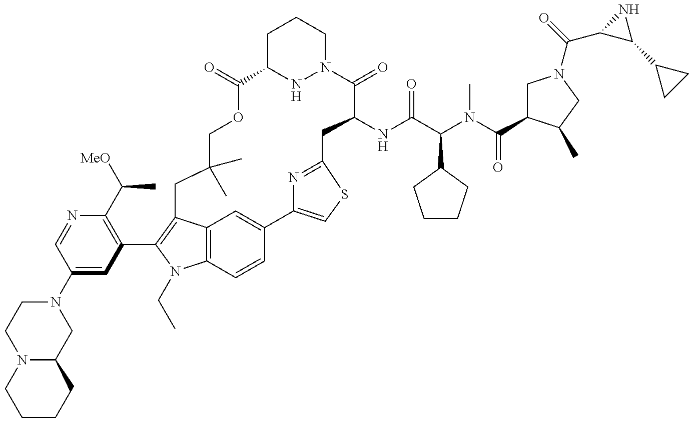 |
| FE 4 | 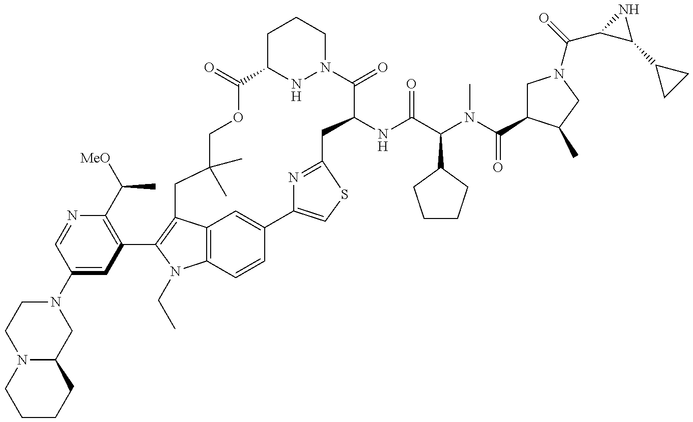 |

In some embodiments, the RAS(ON) inhibitor is a compound selected from Table F6, or a pharmaceutically acceptable salt or stereoisomer thereof. In some embodiments the RAS(ON) inhibitor is a compound selected from Table F6, or a pharmaceutically acceptable salt or atropisomer thereof.

TABLE F6
Certain Compounds of the Present Invention

| Ex # | Structure |
|---|---|
| FF 1 | 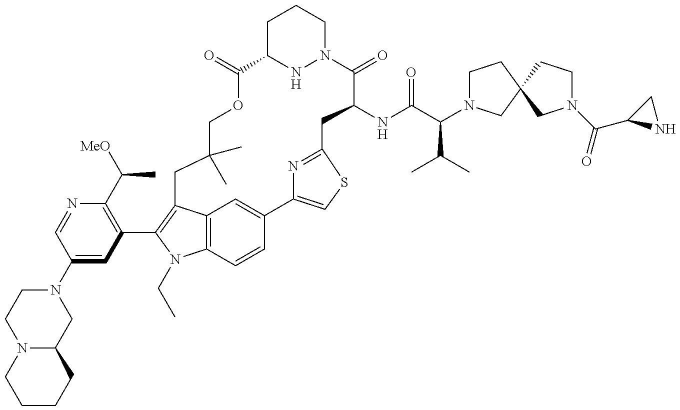 |
| FF 2 | 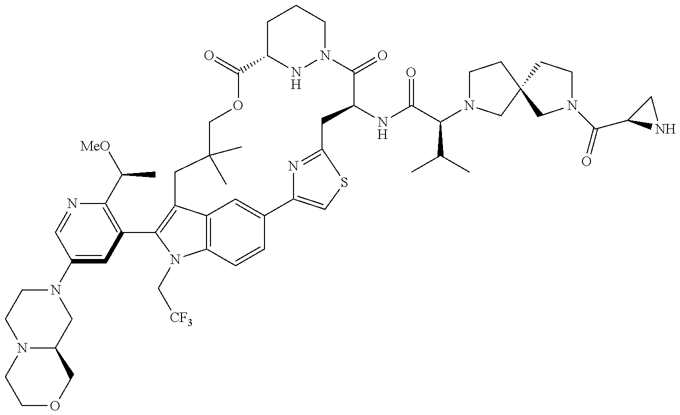 |
| FF 3 | 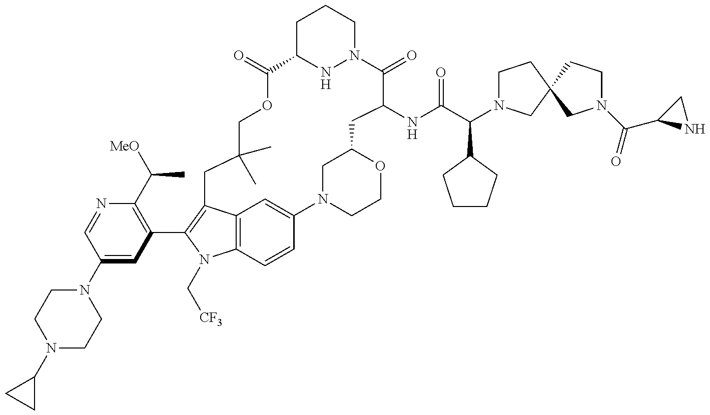 |
| FF 4 | 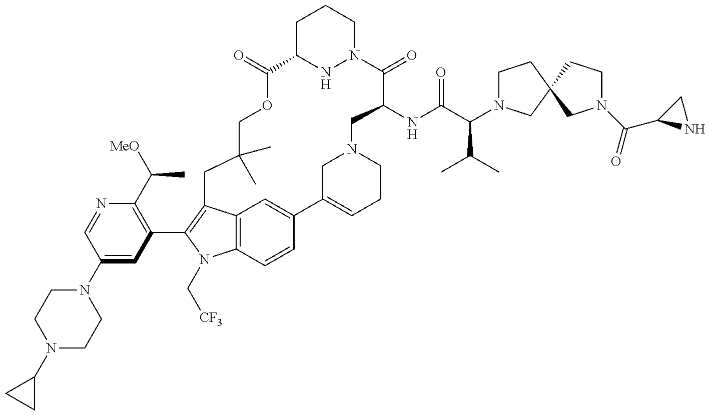 |
| FF 5 | 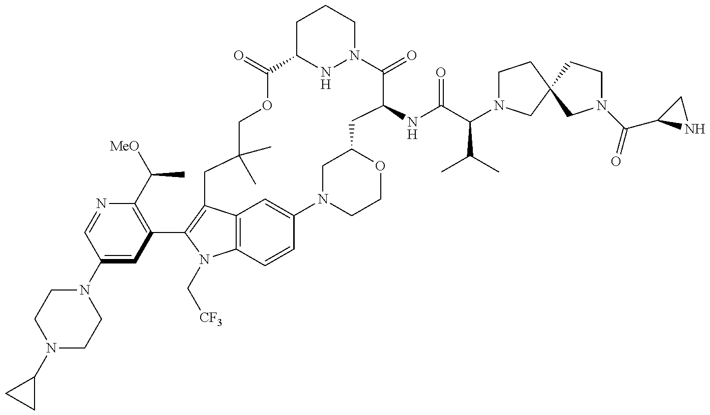 |
| FF 6 | 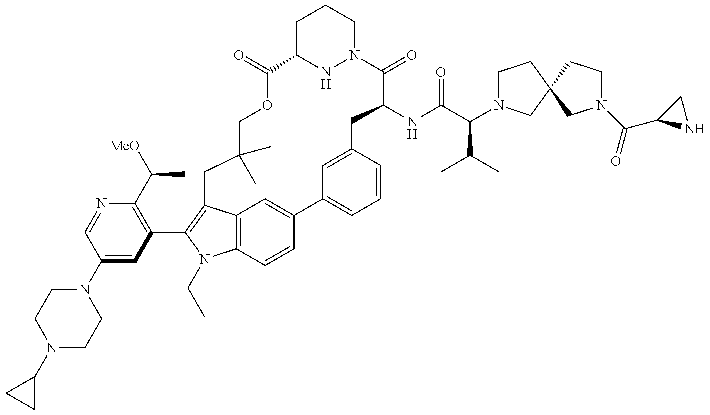 |
| FF 7 | 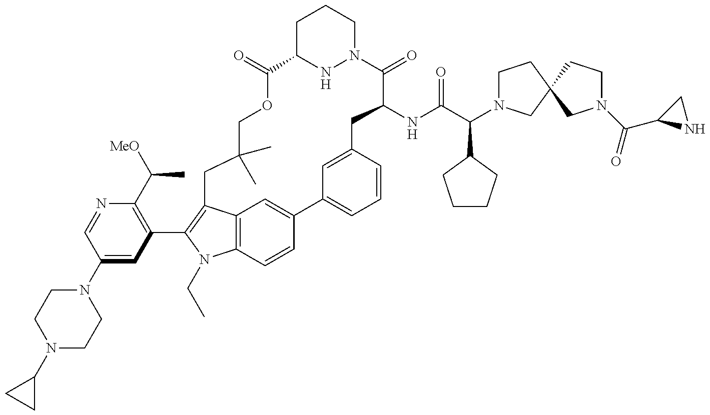 |
| FF 8 | 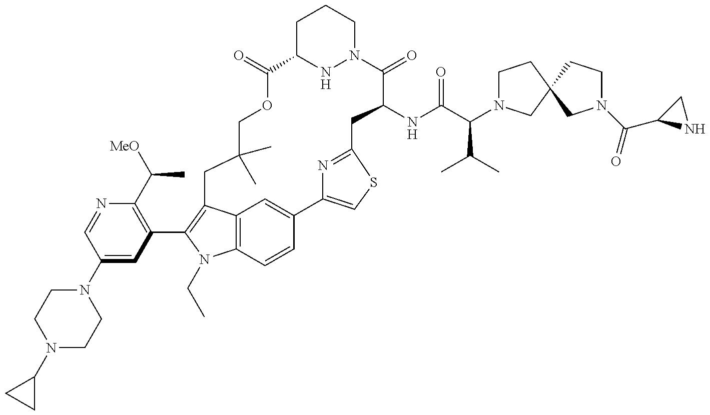 |
| FF 9 | 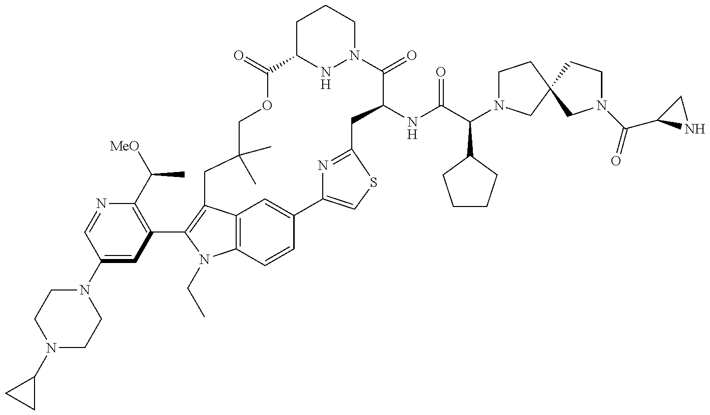 |
| FF 10 | 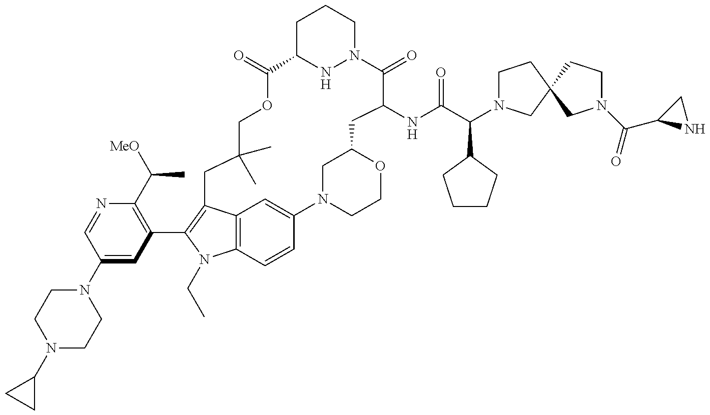 |
| FF 11 | 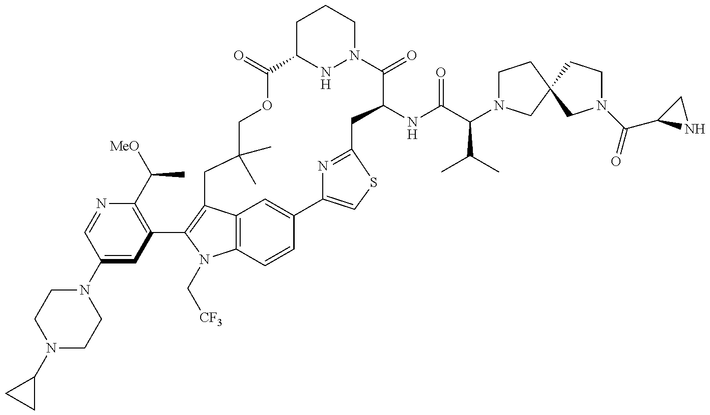 |
| FF 12 | 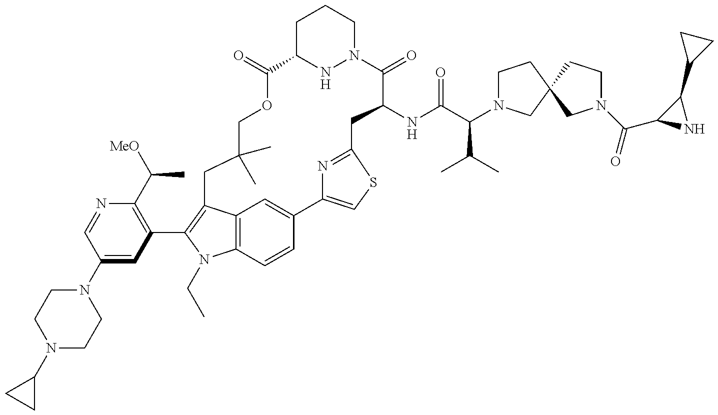 |
| FF 13 | 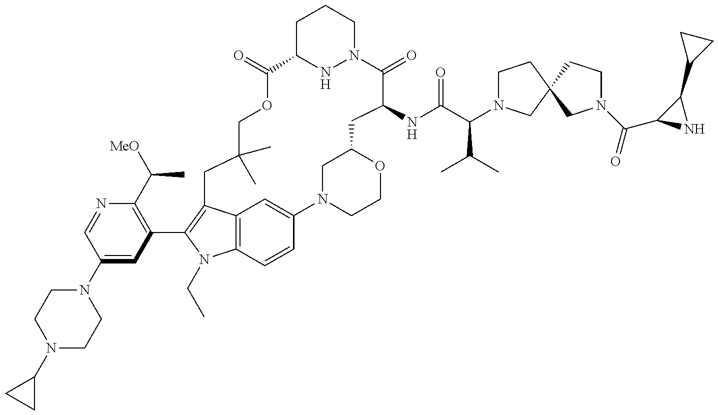 |
| FF 14 | 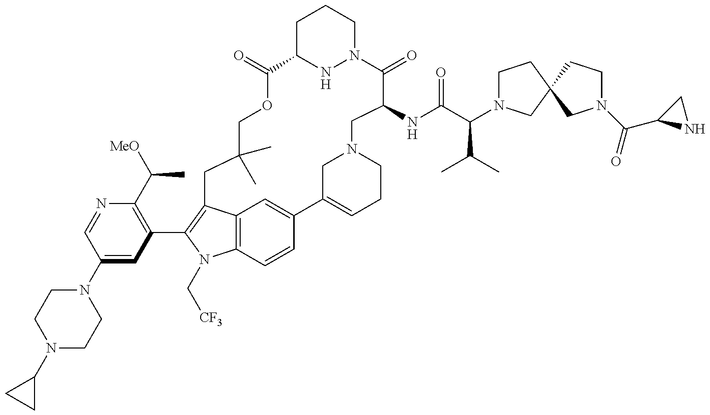 |
| FF 15 | 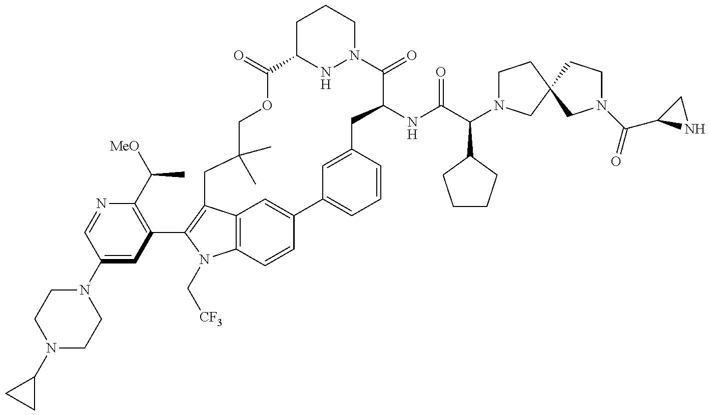 |
| FF 16 | 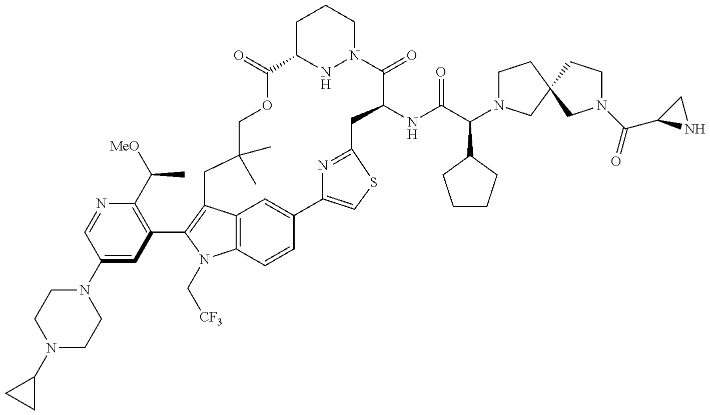 |

* Stereochemistry of the aziridine carbon is assumed.

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated.

In some embodiments of Formula FI or a subformula thereof or Formula FIII or a subformula thereof, W is a cross-linking group bound to an organic moiety that is a Ras binding moiety, i.e., RBM-W, wherein upon contact of an RBM-W compound with a Ras protein, the RBM-W binds to the Ras protein to form a conjugate. For example, the W moiety of an RBM-W compound may bind, e.g., cross-link, with an amino acid of the Ras protein to form the conjugate. In some embodiments, the Ras binding moiety is a K-Ras binding moiety. In some embodiments, the K-Ras binding moiety binds to a residue of a K-Ras Switch-II binding pocket of the K-Ras protein. In some embodiments, the Ras binding moiety is an H-Ras binding moiety that binds to a residue of an H-Ras Switch-II binding pocket of an H-Ras protein. In some embodiments, the Ras binding moiety is an N-Ras binding moiety that binds to a residue of an N-Ras Switch-II binding pocket of an N-Ras protein. The W of an RBM-W compound may comprise any W described herein. The Ras binding moiety typically has a molecular weight of under 1200 Da. See, e.g., see, e.g., Johnson et al., 292:12981-12993 (2017) for a description of Ras protein domains, incorporated herein by reference.

In some embodiments, the RAS(ON) inhibitor is or acts as a prodrug, such as with respect to administration to a cell or to a subject in need thereof.

Also provided are pharmaceutical compositions comprising a compound of the present invention, or a pharmaceutically acceptable salt thereof, and a pharmaceutically acceptable excipient.

In some embodiments, the RAS(ON) inhibitor is provided as a conjugate, or salt thereof, comprising the structure of Formula FIV:

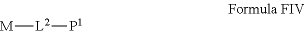

wherein L² is a linker;
P¹ is a monovalent organic moiety; and
M has the structure of Formula FV:

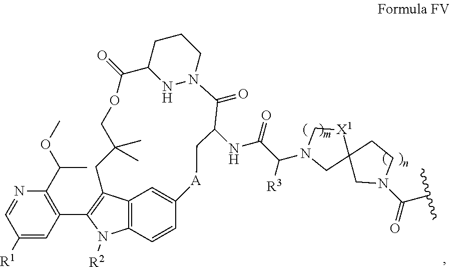

wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
X¹ is CH₂ or O;
m is 1 or 2;
n is 0 or 1;
R¹ is hydrogen or optionally substituted 3 to 10-membered heterocycloalkyl;
R² is optionally substituted C₁-C₆ alkyl; and
R³ is optionally substituted C₁-C₆ alkyl or optionally substituted 3 to 6-membered cycloalkyl.

In some embodiments of conjugates of Formula FIV, the monovalent organic moiety is a protein. In some embodiments, the protein is a Ras protein. In some embodiments, the Ras protein is K-Ras G12D or K-Ras G13D. In some embodiments of conjugates of the present invention, the linker, L², is bound to the monovalent organic moiety through a bond to a carboxyl group of an amino acid residue of the monovalent organic moiety.

With respect to RAS(ON) inhibitors of the present invention, one stereoisomer may exhibit better inhibition than another stereoisomer. For example, one atropisomer may exhibit inhibition, whereas the other atropisomer may exhibit little or no inhibition.

The RAS(ON) inhibitors described herein of Formula FI or a subformula thereof or Formula FIII or a subformula thereof may be made from commercially available starting materials or synthesized using known organic, inorganic, or enzymatic processes.

The RAS(ON) inhibitors can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

Compounds of Table F1, Table F2, Table F3, Table F4, and Table F6 herein were prepared using methods disclosed herein or were prepared using methods disclosed herein combined with the knowledge of one of skill in the art. Compounds of Table F5 may be prepared using methods disclosed herein or may be prepared using methods disclosed herein combined with the knowledge of one of skill in the art.

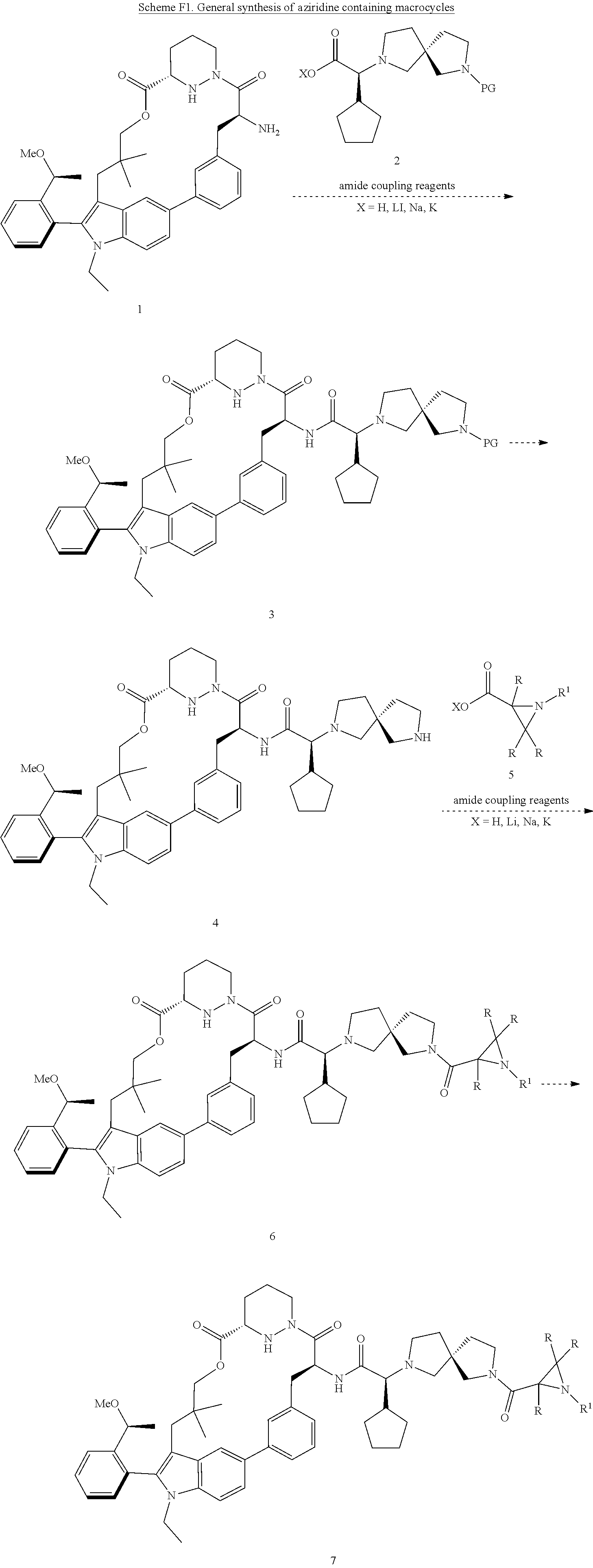

As shown in Scheme F1, compounds of this type may be prepared by the reaction of an appropriate amine (1) with a carboxylic acid containing protected amine (2) in the presence of standard amide coupling reagents to give 3, followed by deprotection of the amine to produce 4. Coupling of an aziridine carboxylate (5) in the presence of standard amide coupling reagents affords 6. If R¹ is a protecting group, deprotection affords the final compound (7).

In some embodiments, the RAS(ON) inhibitor is a compound described by a Formula in WO 2020132597, such as a compound of Formula (I) therein, or a pharmaceutically acceptable salt thereof, or FIG. 1 therein, or a pharmaceutically acceptable salt thereof.

Without being bound by theory, both covalent and non-covalent interactions of a RAS(ON) inhibitor described herein with Ras and the chaperone protein (e.g., cyclophilin A) may contribute to the inhibition of Ras activity. In some embodiments, a RAS(ON) inhibitor described herein forms a covalent adduct with a side chain of a Ras protein (e.g., a sulfhydryl side chain of the cysteine at position 12 or 13 of a mutant Ras protein). Covalent adducts may also be formed with other side chains of Ras. In addition, or alternatively, non-covalent interactions may be at play: for example, van der Waals, hydrophobic, hydrophilic and hydrogen bond interactions, and combinations thereof, may contribute to the ability of the compounds of the present invention to form complexes and act as Ras inhibitors. Accordingly, a variety of Ras proteins may be inhibited by RAS(ON) inhibitors described herein (e.g., K-Ras, N-Ras, H-Ras, and mutants thereof at positions 12, 13 and 61, such as G12C, G12D, G12V, G12S, G13C, G13D, and Q61L, and others described herein).

Methods of determining covalent adduct formation are known in the art and are described in, for example, WO 2021/091982 and WO 2021/091967.

### RAS(OFF) Inhibitors

RAS(OFF) inhibitors are provided herein and are known to those of skill in the art. A RAS(OFF) inhibitor refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS(OFF) inhibitors may also bind to or inhibit the GTP-bound, active state of RAS (e.g., with a lower affinity or inhibition constant than for the GDP-bound, inactive state of RAS).

In some embodiments, the RAS(OFF) inhibitor is selective for RAS that includes an amino acid substitution at G12, G13, Q61, or a combination thereof. In some embodiments, the RAS(OFF) inhibitor is selective for RAS that includes an amino acid substitution selected from G12C, G12D, G12V, G13C, G13D, Q61 L, or a combination thereof. In some embodiments, the RAS(OFF) inhibitor is selective for RAS that includes a G12C or G12D amino acid substitution.

In some embodiments, the RAS(OFF) inhibitor is a KRAS(OFF) inhibitor, where a KRAS(OFF) inhibitor refers to an inhibitor that targets, that is, selectively binds to or inhibits the GDP-bound, inactive state of KRAS (e.g., selective over the GTP-bound, active state of KRAS). In some embodiments, the KRAS(OFF) inhibitor is selective for KRAS that includes an amino acid substitution at G12, G13, Q61, A146, K117, L19, Q22, V14, A59, or a combination thereof. In some embodiments, the KRAS(OFF) inhibitor is selective for KRAS that includes an amino acid substitution selected from G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, G13V, or a combination thereof.

In some embodiments, the RAS(OFF) inhibitor is an NRAS(OFF) inhibitor, where an NRAS(OFF) inhibitor refers to an inhibitor that targets, that is, selectively binds to or inhibits the GDP-bound, inactive state of NRAS (e.g., selective over the GTP-bound, active state of NRAS). In some embodiments, the NRAS(OFF) inhibitor is selective for NRAS that includes an amino acid substitution at G12, G13, Q61, P185, A146, G60, A59, E132, E49, T50, or a combination thereof. In some embodiments, the NRAS(OFF) inhibitor is selective for NRAS that includes an amino acid substitution selected from Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, A59T, or a combination thereof.

In some embodiments, the RAS(OFF) inhibitor is an HRAS(OFF) inhibitor, where an HRAS(OFF) inhibitor refers to an inhibitor that targets, that is, selectively binds to or inhibits the GDP-bound, inactive state of HRAS (e.g., selective over the GTP-bound, active state of HRAS). In some embodiments, the HRAS(OFF) inhibitor is selective for HRAS that includes an amino acid substitution at G12, G13, Q61, K117, A59, A18, D119, A66, A146, or a combination thereof. In some embodiments, the HRAS(OFF) inhibitor is selective for NRAS that includes an amino acid substitution selected from Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, G12R, or a combination thereof.

In some embodiments, the RAS(OFF) inhibitor is a compound disclosed in any one of the following patent publications: WO 2022095960, WO 2022098625, WO 2022093856, WO 2022089219, WO 2022087624, WO 2022 087375, WO 2022087371, WO 2022083616, WO 2022083569, WO 2022081655, WO 2022076917, WO 2022072783, WO 2022066805, WO 2022066646, WO 2022063297, WO 2022061251, WO 2022056307, WO 2022052895, WO 2022048545, WO 2022047093, WO 2022042630, WO 2022040469, WO 2022037631, WO 2022037560, WO 2022031678, WO 2022028492, WO 2022028346, WO 2022026726, WO 2022026723, WO 2022015375, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021088458, WO 2021086833, WO 2021085653, WO 2021084765, WO 2021081212, WO 2021058018, WO 2021057832, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, CN 114437084, CN 114195788, CN 114437107, CN 114409653, CN 114380827, CN 114195804, CN 114057776, CN 114057744, CN 114057743, CN 113999226, CN 113980032, CN 113980014, CN 113960193, CN 113929676, CN 113754653, CN 113683616, CN 113563323, CN 113527299, CN 113527294, CN 113527293, CN 113493440, CN 113429405, CN 113248521, CN 113321654, CN 113087700, CN 113024544, CN 113004269, CN 112920183, CN 112778284, CN 112390818, CN 112390788, CN 112300196, CN 112300194, CN 112300173, CN 112225734, CN 112142735, CN 112110918, CN 112094269, CN 112047937, and CN 109574871, each of which is incorporated herein by reference in its entirety.

In some embodiments, the RAS(OFF) inhibitor is selected from sotorasib (AMG 510), adagrasib (MRTX849), MRTX1257, JNJ-74699157 (ARS-3248), LY3537982, LY3499446, ARS-853, ARS-1620, GDC-6036, JDQ443, BPI-421286, JAB-21000, RSC-1255, ERAS-3490, D-1553, JAB-21822, GH-35, ICP-915, 1B1351, and B11823911.

Reference to “AMG 510,” “MRTX849,” “MRTX1257,” “ARS-853”, “ARS-1620” and GDC-6036 means the following compounds:

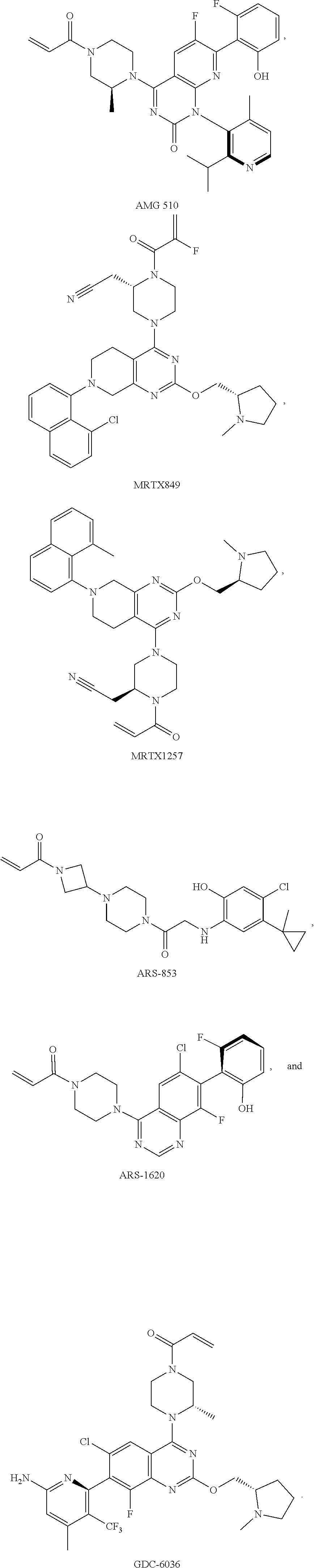

### Pharmaceutical Compositions

The disclosure provides pharmaceutical compositions including one or more RAS inhibitor compounds, or a pharmaceutically acceptable salt thereof, and a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Compounds described herein, whether expressly stated or not, may be provided or utilized in salt form, e.g., a pharmaceutically acceptable salt form, unless expressly stated to the contrary.

The compounds of the disclosure may have ionizable groups so as to be capable of preparation as pharmaceutically acceptable salts. These salts may be acid addition salts involving inorganic or organic acids or the salts may, in the case of acidic forms of the compounds of the disclosure, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

For use as treatment of subjects, the compounds of the disclosure, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present disclosure, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the disclosure, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present disclosure can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the disclosure, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further include an additional compound having antiproliferative (e.g., anti-cancer) activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present disclosure can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Methods of Use

In some embodiments, the disclosure provides a method of treating a disease or disorder that is characterized by aberrant RAS activity due to one or more RAS mutations. In some embodiments, the disease or disorder is a cancer (e.g., a cancer having one or more RAS mutations that cause aberrant RAS activity).

As described herein, cancer cells treated with a RAS(OFF) inhibitor may develop resistance, e.g., through the acquisition of one or more mutations that render the RAS(OFF) inhibitor less effective or ineffective. The present disclosure is based, at least in part, on the observation that some cancers resistant to treatment with a RAS(OFF) inhibitor remain responsive to treatment with a RAS(ON) inhibitor. Thus, administering a RAS(ON) inhibitor to a subject having cancer can slow or halt oncogenic signaling or disease progression where the cancer is resistant to treatment with a RAS(OFF) inhibitor. Additionally, administration of a RAS(ON) inhibitor, e.g., administered in combination with a RAS(OFF) inhibitor, may prevent the acquisition of one or more mutations in RAS that confer resistance to the RAS(OFF) inhibitor. In addition, compounds disclosed herein may provide a clinical benefit for patients naïve to RAS(OFF) therapy.

Accordingly, the disclosure provides a method of treating cancer in a subject in need thereof, the method including administering to the subject a therapeutically effective amount of one or more compounds described here, or a pharmaceutically acceptable salt thereof, or a pharmaceutical composition including one or more compounds described herein or salts thereof.

The disclosure also provides a method of treating cancer in a subject in need thereof, wherein the cancer includes a mutation in RAS and the cancer is resistant to treatment with a RAS(OFF) inhibitor, the method including administering to the subject a RAS(ON) inhibitor.

In some embodiments, the method further includes administering to the subject a RAS(OFF) inhibitor (e.g., a RAS(OFF) inhibitor is administered to the subject in combination with the RAS(ON) inhibitor). The RAS(ON) inhibitor and the RAS(OFF) inhibitor may be administered simultaneously or sequentially. The RAS(ON) inhibitor and the RAS(OFF) inhibitor may be administered as a single formulation or in separate formulations. In some embodiments, the RAS(OFF) inhibitor is administered for a first period of time; and the RAS(ON) inhibitor is administered for a second period of time, wherein the first period of time and the second period of time do not overlap and the first period of time precedes the second period of time. In some embodiments, the RAS(OFF) inhibitor is administered for a first period of time; and the RAS(OFF) inhibitor and RAS(ON) inhibitor are administered for a second period of time, wherein the first period of time and the second period of time do not overlap and the first period of time precedes the second period of time. In some embodiments, the first period of time is a period of time sufficient to acquire a mutation (e.g., a RAS mutation) that confers resistance to treatment with the RAS(OFF) inhibitor. In some embodiments, the first period of time is between one week and one month, between one week and six months, between one week and one year, between one month and six months, between one month and one year, between one month and two years, between one month and five years, at least one week, at least one month, at least six months, or at least one year. In some embodiments, the second period of time is between one week and one month, between one week and six months, between one week and one year, between one month and six months, between one month and one year, between one month and two years, between one month and five years, at least one week, at least one month, at least six months, or at least one year.

In some embodiments, the subject's cancer progresses on the RAS(OFF) inhibitor (e.g., when the subject is administered the RAS(OFF) inhibitor in the absence of a RAS(ON) inhibitor). Disease progression of a cancer (e.g., a cancer described herein) can be evaluated by any one or more of several established methods. A person of skill in the art can monitor a subject by direct observation in order to evaluate how the symptoms exhibited by the subject have changed (e.g., a decrease or absence of symptoms) in response to a treatment (e.g., a method of treatment disclosed herein). A subject may also be examined by MRI, CT scan, or PET analysis in order to determine if a tumor has metastasized or if the size of a tumor has changed (e.g., decreased in response to a treatment (e.g., a method of treatment described herein)). Optionally, cells can be extracted from the subject through a biopsy or procedure or tumor DNA can be isolated from the blood of a subject, and a quantitative biochemical analysis can be conducted in order to assess the relative cancer burden and determine the presence or emergence of specific mutations possibly involved in resistance. Based on the results of these analyses, a person of skill in the art may prescribe higher/lower dosages or more/less frequent dosing of a treatment in subsequent rounds of treatment.

In some embodiments, the subject has been treated with a RAS(OFF) inhibitor (e.g., the subject has been previously treated with a RAS(OFF) inhibitor, e.g., prior to administration of the RAS(ON) inhibitor). In some embodiments, the subject has acquired resistance to a RAS(OFF) inhibitor (e.g., has acquired a mutation that confers resistance to a RAS(OFF) inhibitor, e.g., prior to administration of the RAS(ON) inhibitor).

In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. In some embodiments, the cancer is non-small cell lung cancer. In some embodiments, the cancer is pancreatic cancer.

In some embodiments, the compounds of the present disclosure or pharmaceutically acceptable salts thereof, pharmaceutical compositions including such compounds or salts, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions including such compounds or salts, and methods of the disclosure include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms), multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

In some embodiments, the cancer includes a RAS mutation, such as a RAS mutation described herein. In some embodiments, a mutation is selected from:
1. the following KRAS mutants: G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V, and combinations thereof;
2. the following HRAS mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
3. the following NRAS mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof;
   or a combination of any of the foregoing. In some embodiments, the cancer includes a KRAS mutation selected from the group consisting of G12C, G12D, G13C, G12V, G13D, G12R, G12S, Q61H, Q61K and Q61 L. In some embodiments, the cancer includes an NRAS mutation selected from the group consisting of G12C, Q61H, Q61K, Q61L, Q61P and Q61R. In some embodiments, the cancer includes an HRAS mutation selected from the group consisting of Q61H and Q61 L. In some embodiments, the cancer includes a RAS mutation selected from the group consisting of G12C, G13C, G12A, G12D, G13D, G12S, G13S, G12V and G13V. In some embodiments, the cancer includes at least two RAS mutations selected from the group consisting of G12C, G13C, G12A, G12D, G13D, G12S, G13S, G12V and G13V. In some embodiments, a compound of the present disclosure inhibits more than one RAS mutant. For example, a compound may inhibit both KRAS G12C and KRAS G13C. A compound may inhibit both NRAS G12C and KRAS G12C. In some embodiments, a compound may inhibit both KRAS G12C and KRAS G12D.

In some embodiments, a compound may inhibit both KRAS G12V and KRAS G12C. In some embodiments, a compound may inhibit both KRAS G12V and KRAS G12S. In some embodiments, a compound of the present disclosure inhibits RAS^{amp} in addition to one or more additional RAS mutations (e.g., K-, H- or NRAS^{amp} and KRAS G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V; K-, H- or NRAS^{amp} and HRAS Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K-, H- or NRAS^{amp} and NRAS Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T).

In some embodiments, the cancer is non-small cell lung cancer and the RAS mutation includes a KRAS mutation, such as KRAS G12C, KRAS G12V or KRAS G12D. In some embodiments, the cancer is colorectal cancer and the RAS mutation includes a KRAS mutation, such as KRAS G12C, KRAS G12V or KRAS G12D. In some embodiments, the cancer is pancreatic cancer and the RAS mutation includes an KRAS mutation, such as KRAS G12D or KRAS G12V. In some embodiments, the cancer is pancreatic cancer and the RAS mutation includes an NRAS mutation, such as NRAS G12D. In some embodiments, the cancer is melanoma and the RAS mutation includes an NRAS mutation, such as NRAS Q61R or NRAS Q61K.

In some embodiments, a cancer includes a RAS mutation and an STK11^{LOF}, a KEAP1, an EPHA5 or an NF1 mutation. In some embodiments, the cancer is non-small cell lung cancer and includes a KRAS G12C mutation. In some embodiments, the cancer is non-small cell lung cancer and includes a KRAS G12C mutation and an STK11^{LOF} mutation. In some embodiments, the cancer is non-small cell lung cancer and includes a KRAS G12C mutation and an STK11^{LOF} mutation. In some embodiments, a cancer includes a KRAS G13C RAS mutation and an STK11^{LOF}, a KEAP1, an EPHA5 or an NF1 mutation. In some embodiments, the cancer is non-small cell lung cancer and includes a KRAS G12D mutation. In some embodiments, the cancer is non-small cell lung cancer and includes a KRAS G12V mutation. In some embodiments, the cancer is colorectal cancer and includes a KRAS G12C mutation. In some embodiments, the cancer is pancreatic cancer and includes a KRAS G12D mutation. In some embodiments, the cancer is pancreatic cancer and includes a KRAS G12V mutation. In some embodiments, the cancer is endometrial cancer and includes a KRAS G12C mutation. In some embodiments, the cancer is gastric cancer and includes a KRAS G12C mutation.

Methods for detecting a mutation in a KRAS, HRAS or NRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12C KRAS, HRAS or NRAS mutations by real-time PCR. In real-time PCR, fluorescent probes specific for the KRAS, HRAS or NRAS G12C mutation are used. When a mutation is present, the probe binds and fluorescence is detected. In some embodiments, the KRAS, HRAS or NRAS G12C mutation is identified using a direct sequencing method of specific regions (e.g., exon 2 or exon 3) in the KRAS, HRAS or NRAS gene. This technique will identify all possible mutations in the region sequenced.

Methods for detecting a mutation in a KRAS, HRAS or NRAS protein are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing. Other methods include ctDNA measurement (e.g., Cescon et al., Nature Cancer 1:276-290 (2020)), and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

Methods for determining whether a tumor or cancer includes a G12C or other KRAS, HRAS or NRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is a circulating tumor cell (CTC) sample. In some embodiments, the sample is processed to a cell lysate. In some embodiments, the sample is processed to DNA or RNA.

Also provided is a method of inhibiting a RAS protein in a cell, the method including contacting the cell with an effective amount of a compound of the present disclosure, or a pharmaceutically acceptable salt thereof. The cell may be a cancer cell. The cancer cell may be of any type of cancer described herein. The cell may be in vivo or in vitro.

### Combination Therapies

The methods of the disclosure may include a compound of the disclosure used alone or in combination with one or more additional therapies (e.g., non-drug treatments or therapeutic agents).

In particular, the disclosure provides methods of treatment that include administering (e.g., to a subject or a cell) a RAS(ON) inhibitor with one or more additional therapies (e.g., one or more additional cancer therapies described herein). In some embodiments, a RAS(ON) inhibitor is administered in combination with a RAS(OFF) inhibitor. In some embodiments, a RAS(ON) inhibitor is administered in combination with a RAS(OFF) inhibitor and one or more additional therapies (e.g., one or more additional cancer therapies described herein).

As described herein, “in combination,” includes administration of two or more therapies as part of a therapeutic regimen. The therapies may be administered simultaneously or sequentially. Such sequential administration may be close or remote in time. Where the therapies are therapeutic agents, the therapeutic agents may be formulated together as a single dosage form or formulated as separate dosage forms. The therapeutic agents may be administered by the same route of administration or by different routes of administration.

When a RAS(ON) inhibitor is administered in combination with one or more additional therapies, the RAS(ON) inhibitor may be administered before, after, or concurrently with one or more of such additional therapies.

The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment. For example, in some embodiments, the compounds of the present disclosure can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

In this combination therapy section, all references are incorporated by reference for the agents described, whether explicitly stated as such or not.

### Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the compounds of the disclosure may be used as an adjuvant therapy after surgery. In some embodiments, the compounds of the disclosure may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachy therapy,” as used herein, refers to radiation therapy delivered by a spatially confined radioactive material inserted into the body at or near, a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present disclosure can render abnormal cells more sensitive to treatment with radiation for purposes of killing or inhibiting the growth of such cells. Accordingly, this disclosure further relates to a method for sensitizing abnormal cells in a mammal to treatment with radiation which includes administering to the mammal an amount of a compound of the present disclosure, which amount is effective to sensitize abnormal cells to treatment with radiation. The amount of the compound in this method can be determined according to the means for ascertaining effective amounts of such compounds described herein. In some embodiments, the compounds of the present disclosure may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### Therapeutic Agents

A therapeutic agent may be a compound used in the treatment of cancer or symptoms associated therewith.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present disclosure include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PDL-1. In some embodiments, the checkpoint inhibitor is an inhibitor or antagonist (e.g., an inhibitory antibody or Fc fusion or small molecule inhibitor) of PDL-2 (e.g., a PDL-2/lg fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. Nat. Rev. Neurol. 11(9):504-514 (2015), including, without limitation, ipilimumab, tremelimumab, nivolumab, pembrolizumab, AMP224, AMP514/MEDI0680, BMS936559, MED14736, MPDL3280A, MSB0010718C, BMS986016, IMP321, lirilumab, IPH2101, 1-7F9, and KW-6002.

A therapeutic agent may be an anti-TIGIT antibody, such as MBSA43, BMS-986207, MK-7684, COM902, AB154, MTIG7192A or OMP-313M32 (etigilimab).

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezornib); Casodex (bicalutarnide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, Ianiquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK4/6 inhibitor such as abemaciclib, ribociclib, palbociclib; seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., vistusertib, temsirolimus, everolimus, ridaforolimus, and sirolimus), KSP(Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TGO2 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), P13K/Akt inhibitors (e.g., perifosine), Akt inhibitors (e.g., GSK-2141795), PKC inhibitors (e.g., enzastaurin), FTIs (e.g., Zamestra™), anti-CD138 (e.g., BT062), Torc1/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, the anti-cancer agent is a HER2 inhibitor. Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, JAB-3312, RLY-1971, BBP-398, ERAS-601, PF-07284892, SH3809; see also Wu et al., Curr Med Chem (2020) 27:1; world wide web at doi.org/10.2174/1568011817666200928114851), a SOS1 inhibitor (e.g., BI-1701963, BI-3406, SDR5, BAY-293, RMC-5845, MRTX0902), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312.

In some embodiments, an anti-cancer agent is a SOS1 inhibitor. In some embodiments, the SOS1 inhibitor is selected from those disclosed in WO 2022081912, WO 2022058344, WO 2022028506, WO 2022026465, WO 2022017339, WO 2022017519, WO 2021249519, WO 2021249575, WO 2021228028, WO 2021225982, WO 2021203768, WO 2021173524, WO 2021130731, WO 2021127429, WO 2021092115, WO 2021105960, WO 2021074227, WO 2020180768, WO 2020180770, WO 2020173935, WO 2020146470, WO 2019201848, WO 2019122129, WO 2018172250, WO 2018115380, CN 114456165, CN 114436976, CN 114380805, CN 113912608, CN 1138010114, CN 113200981, and US 20210338694, or a pharmaceutically acceptable salt, solvate, isomer (e.g., stereoisomer), prodrug, or tautomer thereof.

In some embodiments, a therapeutic agent that may be combined with a compound of the present disclosure is an inhibitor of the MAP kinase (MAPK) pathway (or “MAPK inhibitor”). MAPK inhibitors include, but are not limited to, one or more MAPK inhibitor described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9(11)); and GSK1120212 (or JTP-74057, described in Clin Cancer Res. 2011 Mar. 1; 17(5):989-1000). The MAPK inhibitor may be PLX8394, LXH254, GDC-5573, or LY3009120.

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies. In some embodiments, a therapeutic agent may be a pan-RTK inhibitor, such as afatinib.

IGF-1R inhibitors include linsitinib, or a pharmaceutically acceptable salt thereof.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. In some embodiments, the EGFR inhibitor is osimertinib (Tagrisso®). Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8(12):1599-1625. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4).

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N.

P13K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl] phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other P13K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552, or a stereoisomer, tautomer, or oxepane isomer thereof, or pharmaceutically acceptable salt of any of the foregoing.

BRAF inhibitors that may be used in combination with compounds of the disclosure include, for example, vemurafenib, dabrafenib, and encorafenib. A BRAF may include a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581 I; D594N; D594G; D594A; D594H; F595L; G596D; G596R and A762E.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is a SHP2 inhibitor. SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N-SH2 and C-SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N-SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phosphoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) have been shown to inhibit the proliferation of multiple cancer cell lines in vitro (e.g., pancreas, lung, ovarian and breast cancer). Thus, combination therapy involving a SHP2 inhibitor with a RAS pathway inhibitor could be a general strategy for preventing tumor resistance in a wide range of malignancies.

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; Wu et al., Curr Med Chem (2020) 27:1; world wide web at doi.org/10.2174/1568011817666200928114851; and patent publications: WO 2022089406, WO 2022089389, WO 2022063190, WO 2022043685, WO 2022042331, WO 2022033430, WO 2022033430, WO 2022017444, WO 2022007869, WO 2021259077, WO 2021249449, WO 2021249057, WO 2021244659, WO 2021218755, WO 2021281752, WO 2021197542, WO 2021176072, WO 2021149817, WO 2021148010, WO 2021147879, WO 2021143823, WO 2021143701, WO 2021143680, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, US 20210085677, U.S. Ser. No. 10/858,359, U.S. Ser. No. 10/934,302, U.S. Ser. No. 10/954,243, U.S. Ser. No. 10/988,466, U.S. Ser. No. 11/001,561, U.S. Ser. No. 11/033,547, U.S. Ser. No. 11/034,705, U.S. Ser. No. 11/044,675, CN 114213417, CN 114163457, CN 113896710, CN 113248521, CN 113248449, CN 113135924, CN 113024508, CN 112920131, CN 112823796, CN 112402385, CN 111848599, CN 111704611, CN 111265529, and CN 108113848, each of which is incorporated herein by reference.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C₃₃₃) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TNO155, or a pharmaceutically acceptable salt, solvate, isomer (e.g., stereoisomer), prodrug, or tautomer thereof. In some embodiments, the SHP2 inhibitor is RMC-4550, or a pharmaceutically acceptable salt, solvate, isomer (e.g., stereoisomer), prodrug, or tautomer thereof. In some embodiments, the SHP2 inhibitor is RMC-4630, or a pharmaceutically acceptable salt, solvate, isomer (e.g., stereoisomer), prodrug, or tautomer thereof. In some embodiments, the SHP2 inhibitor is JAB-3068, or a pharmaceutically acceptable salt, solvate, isomer (e.g., stereoisomer), prodrug, or tautomer thereof. In some embodiments, the SHP2 inhibitor is JAB-3312. In some embodiments, the SHP2 inhibitor is the following compound

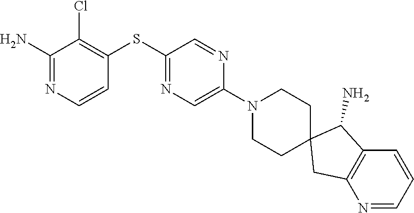 [SMILES: Nc1nccc(Sc2cnc(N3CCC4(CC3)Cc3ncccc3[C@H]4N)cn2)c1Cl]

or a pharmaceutically acceptable salt, solvate, isomer (e.g., stereoisomer), prodrug, or tautomer thereof. In some embodiments, the SHP2 inhibitor is RLY-1971, or a pharmaceutically acceptable salt, solvate, isomer (e.g., stereoisomer), prodrug, or tautomer thereof.

In some embodiments, the SHP2 inhibitor is ERAS-601, or a pharmaceutically acceptable salt, solvate, isomer (e.g., stereoisomer), prodrug, or tautomer thereof. In some embodiments, the SHP2 inhibitor is BBP-398, or a pharmaceutically acceptable salt, solvate, isomer (e.g., stereoisomer), prodrug, or tautomer thereof. In some embodiments, the SHP2 inhibitor is SH3809. In some embodiments, the SHP2 inhibitor is PF-07284892, or a pharmaceutically acceptable salt, solvate, isomer (e.g., stereoisomer), prodrug, or tautomer thereof.

In some embodiments, the additional therapeutic agent is selected from the group consisting of a MEK inhibitor, a HER2 inhibitor, a SHP2 inhibitor, CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a RAS inhibitor of the present disclosure is used in combination with a MEK inhibitor and a SOS1 inhibitor. In some embodiments, a RAS inhibitor of the present disclosure is used in combination with a PDL-1 inhibitor and a SOS1 inhibitor. In some embodiments, a RAS inhibitor of the present disclosure is used in combination with a PDL-1 inhibitor and a SHP2 inhibitor. In some embodiments, a RAS inhibitor of the present disclosure is used in combination with a MEK inhibitor and a SHP2 inhibitor. In some embodiments, the cancer is colorectal cancer and the treatment includes administration of a RAS inhibitor of the present disclosure in combination with a second or third therapeutic agent.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PDL-1, anti-CTLA4, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the disclosure is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists(ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the disclosure include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the disclosure is an autophagy inhibitor. Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the disclosure is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-la, interferon beta-Ib, interferon gamma, natural interferon gamma-la, interferon gamma-Ib, interleukin-1 beta, iobenguane, irinotecan, irsogladine, Ianreotide, LC 9018 (Yakult), leflunomide, lenograstim, lentinan sulfate, letrozole, leukocyte alpha interferon, leuprorelin, levamisole+fluorouracil, liarozole, lobaplatin, Ionidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the disclosure include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the disclosure and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the disclosure and any of the therapies described herein are administered a few minutes apart, or a few hours apart, or a few days apart.

In some embodiments of any of the methods described herein, the first therapy (e.g., a compound of the disclosure) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The disclosure also features kits including (a) a pharmaceutical composition including an agent (e.g., one or more compounds of the disclosure) described herein, and (b) a package insert with instructions to perform any of the methods described herein. In some embodiments, the kit includes (a) a pharmaceutical composition including an agent (e.g., one or more compounds of the disclosure) described herein, (b) one or more additional therapies (e.g., non-drug treatment or therapeutic agent), and (c) a package insert with instructions to perform any of the methods described herein.

As one aspect of the present disclosure contemplates the treatment of the disease or symptoms associated therewith with a combination of pharmaceutically active compounds that may be administered separately, the disclosure further relates to combining separate pharmaceutical compositions in kit form. The kit may include two separate pharmaceutical compositions: a compound of the present disclosure, and one or more additional therapies. The kit may include a container for containing the separate compositions such as a divided bottle or a divided foil packet. Additional examples of containers include syringes, boxes, and bags. In some embodiments, the kit may include directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1. Compound AA, a KRAS^{G12C}(ON) Inhibitor Disclosed Herein, is Active Against Second Site Mutations Conferring Resistance to KRAS^{G12C}(OFF) Inhibitors MRTX849 and AMG 510

FIG. 1 is a heatmap representing cellular RAS/RAF disruption assay results regarding various KRAS mutations in the presence of different RAS inhibitors. Certain mutations have been observed in patients treated with AMG 510 (e.g., Y96C, Y96D, H95D, H95Q, H95R, R68S) (Tanaka et al., Clinical acquired resistance to KRASG12C inhibition through a novel KRAS switch-II pocket mutation and polyclonal alterations converging on RAS-MAPK reactivation, Cancer Discovery, Apr. 6, 2021. DOI: 10.1158/2159-8290. CD-21-0365; Awad et al., Mechanisms of acquired resistance to KRAS^{G12C} inhibition in cancer, AACR Annual Meeting 2021, Apr. 10, 2021).

Plasmids expressing nanoluciferase-tagged mutant KRAS4B and halo-tagged RAF1(residues 51-149) were co-transfected into U2OS cells and incubated for 24 hours. Plasmids encoding the relevant mutation were generated by New England Biolabs Q5 site-directed mutagenesis. Transfected cells were reseeded at 25000 cells/well in 96-well plates in assay media (OptiMEM+4% FBS+100 nM HaloTag NanoBRET 618 Ligand) and incubated overnight. Promega Vivazine Nano-Glo substrate was added according to manufacturer's instructions. Compounds were added at concentrations ranging from 0 to 10 μM and incubated for 5 hours. The luminescence signal was measured at 460 nm and 618 nm and the BRET ratio was calculated as the 618 nm signal divided by the 460 nm signal. The BRET ratios were fit to a standard sigmoidal dose response function and the IC50 values were used to calculate the Log₂(Fold-Change) relative to KRAS^{G12C}. FIG. 1B shows the IC50 value associated with each colored bar of the heatmap.

### Example 2. Compound A, a KRAS^{MULTI}(ON) Inhibitor Disclosed Herein, is Active Against RAS Oncogene Switching Mutations Observed in KRAS^{G12C}(OFF) Resistance

FIG. 2A is a heatmap representing cellular RAS/RAF disruption assay results regarding various KRAS mutations in the presence of different RAS inhibitors (Compound A, a KRAS^{MULTI}(ON) inhibitor disclosed herein, and KRAS^{G12C}(OFF) inhibitors MRTX849 and AMG 510). Certain mutations have been observed in patients treated with AMG 510 (e.g., G12C, G12F, G12R, G12V, G12W) (Tanaka et al.; Awad et al.).

Plasmids expressing nanoluciferase-tagged mutant KRAS4B and halo-tagged RAF1(residues 51-149) were co-transfected into U2OS cells and incubated for 24 hours. Plasmids encoding the relevant mutation were generated by New England Biolabs Q5 site-directed mutagenesis. Transfected cells were reseeded at 25000 cells/well in 96-well plates in assay media (OptiMEM+4% FBS+100 nM HaloTag NanoBRET 618 Ligand) and incubated overnight. Promega Vivazine Nano-Glo substrate was added according to manufacturer's instructions. Compounds were added at concentrations ranging from 0 to 10 μM and incubated for 1 hour. The luminescence signal was measured at 460 nm and 618 nm and the BRET ratio was calculated as the 618 nm signal divided by the 460 nm signal. The BRET ratios were fit to a standard sigmoidal dose response function and the IC50 values were used to calculate the Log 2(Fold-Change) relative to KRAS^{G12C}. FIG. 2B shows the IC50 value associated with each colored bar of the heatmap.

### Example 3. Compound A Exhibits Potent in Vivo Inhibition of Multiple RAS-Driven Cancer Cell Lines

Methods: Potency of in vitro cell proliferation inhibition of Capan-1 (KRAS^{G}12W), AsPC-1 (KRAS^{G12D}), HCT116 (KRAS^{G13D}), SK-MEL-30 (NRAS^{Q61K}), NCI-H1975 (EGFR^{T790M/L858R}), and A375 (BRAF^{V600E}) cells exposed to Compound A, a KRAS^{MULTI}(ON) inhibitor disclosed herein, for 120 hours. Data represent the mean of multiple experiments. Cells were seeded in growth medium in 384-well assay plates and incubated overnight in a humidified atmosphere of 5% CO₂ at 37° C. The following day, cells were exposed to a 9-concentration 3-fold serial dilution of Compound A at a starting assay concentration of 1 μM (or 10 μM for A375). After 5 days of incubation, CellTiter-Glo® 2.0 Reagent was added to assay plates and luminescence measured. Data were normalized to the mean signal of DMSO-treated cells, and IC₅₀ values were estimated using a four-parameter concentration response model.

Results: Compound A inhibited cell proliferation in RAS-driven lines (FIG. 3). IC50 for RAS-Driven cancer cell lines as follows: Capan-1 (KRAS^{G12V})=1 nM, AsPC-1 (KRAS^{G12D})=3 nM, HCT116 (KRAS^{G13D})=27 nM, SK-MEL-30 (NRAS^{Q61K})=13 nM, NCI-H1975 (EGFR^{T790M/L858R})=1 nM. RAS WT-Independent cell line A375 (BRAF^{V600E}) was not sensitive to Compound A treatment with IC50>8700 nM.

### Example 4. Synthesis of Compounds of Table A1

Compounds of Table A1, and intermediates in the synthesis thereto, were prepared according to experimental procedures detailed in the Example section of WO 2021/091956, which is incorporated herein by reference in its entirety.

### Example 5. Biological Assay Data for Compounds of Table A1

Compounds of Table A1 were tested in the following biological assays as described in detail in WO 2021/091956: decrease in cellular pERK; determination of cell viability in RAS mutant cancer cell lines; disruption of B-Raf Ras-binding domain (BRAFRBD) interaction with K-Ras; and in vivo pharmacodynamic and efficacy.

The corresponding data for compounds of Table A1 evaluated in the assays described above are given in Tables 4-20, FIG. 1A, FIG. 1B, and the Examples section of WO 2021/091956.

### Example 6. Synthesis of Compounds of Table B1

Compounds of Table B1, and intermediates in the synthesis thereto, were prepared according to experimental procedures detailed in the Example section of WO 2021/091982, which is incorporated herein by reference in its entirety.

### Example 7. Biological Assay Data for Compounds of Table B1

Compounds of Table B1 were tested in the following biological assays as described in detail in WO 2021/091982: decrease in cellular pERK; determination of cell viability in RAS mutant cancer cell lines; disruption of B-Raf Ras-binding domain (BRAF^{RBD}) interaction with K-Ras; in vitro cell proliferation panels; in vivo NSCLC K-Ras G12C xenograft models; and a cell proliferation assay. Certain compounds were also tested in a matched-pair analysis, wherein a H was replaced with (S)Me in the context of two different cell-based assays.

The corresponding data for compounds of Table B1 evaluated in the assays described above are given in Tables 4-19, FIG. 1A, FIG. 1B, FIG. 2A, and FIG. 2B, FIG. 3A, FIG. 3B, FIG. 4A, FIG. 4B, FIG. 5, and the Examples section of WO 2021/091982.

### Example 8. Synthesis of Compounds of Table C1

Compounds of Table C1, and intermediates in the synthesis thereto, were prepared according to experimental procedures detailed in the Example section of WO 2021/091967, which is incorporated herein by reference in its entirety.

### Example 9. Biological Assay Data for Compounds of Table C1

Compounds of Table C1 were tested in the following biological assays as described in detail in WO 2021/091967: decrease in cellular pERK; determination of cell viability in RAS mutant cancer cell lines; disruption of B-Raf Ras-binding domain (BRAF^{BRD}) interaction with K-Ras; cross-linking of Ras proteins with compounds to form conjugates; in vitro cell proliferation panels; and in vivo pharmacodynamics and efficacy.

The corresponding date for compounds of Table C1 evaluated in the assays described above are given in Tables 5-20, FIG. 1A, FIG. 1B, and the Examples section of WO 2021/091967.

### Example 10. Synthesis of Compounds of Table D1

Compounds of Table D1, and intermediates in the synthesis thereto, were prepared according to experimental procedures detailed in the Example section of WO 2022/060836, which is incorporated herein by reference in its entirety.

### Example 11. Biological Assay Data for Compounds of Table D1

Compounds of Table D1 were tested in the following biological assays as described in detail in WO 2022/060836: decrease in cellular pERK; disruption of B-Raf Ras-binding domain (BRAF^{BRD}) interaction with K-Ras; determination of cell viability in RAS mutant cancer cell lines; regressions of KRAD^{G12D} tumors in vivo; regulation of RAS pathway and regressions of KRAS^{G12V} tumors in vivo; regressions of KRAS^{G12V} pancreatic ductal adenocarcinoma and colorectal tumors in vivo; in vivo inhibition of multiple RAS-driven cancer call lines; regressions of KRAS^{G12D} tumors in vivo; regulation of immune checkpoint proteins in NCI-H358, SW900, and Capan-2 cells in vitro; activity against RAS oncogene switching mutations; regressions of a syngenic KRAS G12C tumor model in vivo and synergy with anti-PD-1; modulation of the immune tumor microenvironment in favor of anti-tumor immunity in vivo; anti-tumor activity in KRAS^{G12X} tumor models in vivo; extension of time to tumor doubling across xenograft models; regressions of KRAS^{G12V} tumors in vivo; and inhibition of RAS pathway signaling in vivo.

The corresponding data for compounds of Table D1 evaluated in the assays described above are given in Table 5, FIG. 1A, FIG. 1B, FIG. 1C, FIG. 1D, FIG. 1E, FIG. 1F, FIG. 2A, FIG. 2B, FIG. 2C, FIG. 2D, FIG. 2E, FIG. 2F, FIG. 3A, FIG. 3B, FIG. 3C, FIG. 4A, FIG. 4B, FIG. 4C, FIG. 4D, FIG. 4E, FIG. 4F, FIG. 5A, FIG. 5B, FIG. 5C, FIG. 6A-6B, FIG. 7A-7D, FIG. 8, FIG. 9A, FIG. 9B, FIG. 9C, FIG. 10A, FIG. 10B, FIG. 11, FIG. 12A, FIG. 12B, FIG. 12C, FIG. 13A, FIG. 13B, and the Examples section of WO 2022/060836, which is incorporated herein by reference in its entirety.

### Example 12. Chemical Synthesis of Compounds of Table E1

Definitions used in the following examples and elsewhere herein are:

| | |
|---|---|
| CH₂Cl₂, | Methylene chloride, Dichloromethane |
| DCM | |
| CH₃CN, | Acetonitrile |
| MeCN | |
| CuI | Copper (I) iodide |
| DIPEA | Diisopropylethyl amine |
| DMF | N,N-Dimethylformamide |
| EtOAc | Ethyl acetate |
| h | hour |
| H₂O | Water |
| HCl | Hydrochloric acid |
| K₃PO₄ | Potassium phosphate (tribasic) |
| MeOH | Methanol |
| Na₂SO₄ | Sodium sulfate |
| NMP | N-methyl pyrrolidone |
| Pd(dppf)Cl₂ | [1,1′- |
| | Bis(diphenylphosphino)ferrocene]dichloropalladium(II) |

### Instrumentation

Mass spectrometry data collection took place with a Shimadzu LCMS-2020, an Agilent 1260LC-6120/6125MSD, a Shimadzu LCMS-2010EV, or a Waters Acquity UPLC, with either a QDa detector or SQ Detector 2. Samples were injected in their liquid phase onto a C-18 reverse phase. The compounds were eluted from the column using an acetonitrile gradient and fed into the mass analyzer. Initial data analysis took place with either Agilent ChemStation, Shimadzu LabSolutions, or Waters MassLynx. NMR data was collected with either a Bruker AVANCE III HD 400 MHz, a Bruker Ascend 500 MHz instrument, or a Varian 400 MHz, and the raw data was analyzed with either TopSpin or Mestrelab Mnova.

### Synthesis of Intermediates

### Intermediate E1. Synthesis of 3-(5-bromo-1-ethyl-2-[2-[(1S)-1-methoxyethyl]pyridin-3-yl]indol-3-yl)-2,2-dimethylpropan-1-ol

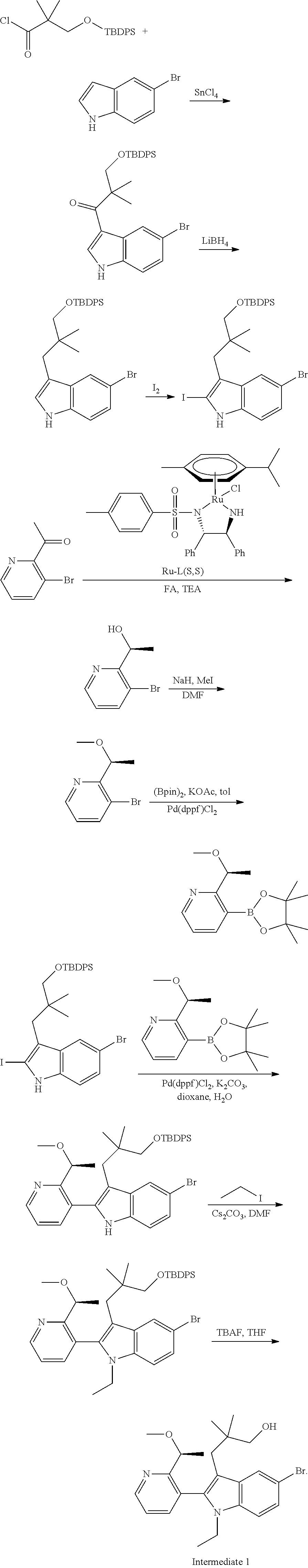

Step 1. To a mixture of 3-((tert-butyldiphenylsilyl)oxy)-2,2-dimethylpropanoyl chloride (65 g, 137 mmol, crude) in DCM (120 mL) at 0° C. under an atmosphere of N₂ was added 1M SnCl₄ in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (100 mL×4), dried over Na₂SO₄, and filtered. The filtrate was concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (55 g, 75% yield). LCMS (ESI): m/z [M+Na] calc'd for C₂₉H₃₂BrNO₂SiNa 556.1; found 556.3.

Step 2. To a mixture of 1-(5-bromo-1H-indol-3-yl)-3-((tert-butyldiphenylsilyl)oxy)-2,2-dimethylpropan-1-one (50 g, 93.6 mmol) in THE (100 mL) at 0° C. under an atmosphere of N₂ was added LiBH₄ (6.1 g, 281 mmol). The mixture was heated to 60° C. and stirred for 20 h, then MeOH (10 mL) and EtOAc (100 mL) were added and the mixture washed with brine (50 mL), dried over Na₂SO₄, filtered, and the filtrate concentrated under reduced pressure. The residue was diluted with DCM (50 mL), cooled to 10° C. and diludine (9.5 g, 37.4 mmol) and TsOH·H₂O (890 mg, 4.7 mmol) added. The mixture was stirred at 10° C. for 2 h, filtered, the filtrate concentrated under reduced pressure and the residue was purified by silica gel column chromatography to give 1-(5-bromo-1H-indol-3-yl)-3-((tert-butyldiphenylsilyl)oxy)-2,2-dimethylpropan-1-one (41 g, 84% yield). LCMS (ESI): m/z [M+H] calc'd for C₂₉H₃₄BrNOSi 519.2; found 520.1; ¹H NMR (400 MHz, CDCl₃) δ 7.96 (s, 1H), 7.75-7.68 (m, 5H), 7.46-7.35 (m, 6H), 7.23-7.19 (m, 2H), 6.87 (d, J=2.1 Hz, 1H), 3.40 (s, 2H), 2.72 (s, 2H), 1.14 (s, 9H), 0.89 (s, 6H).

Step 3. To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THE (15 mL) at rt was added AgOTf (888 mg, 3.5 mmol). The mixture was stirred at rt for 2 h, then diluted with EtOAc (200 mL) and washed with saturated Na₂S₂O₃ (100 mL), dried over anhydrous Na₂SO₄, and filtered. The filtrate was concentrated under reduced pressure and the residue was purified by silica gel column chromatography to give 5-bromo-3-(3-((tert-butyldiphenylsilyl)oxy)-2,2-dimethylpropyl)-2-iodo-1H-indole (900 mg, 72% yield) as a solid. ¹H NMR (400 MHz, DMSO-d₆) δ 11.70 (s, 1H), 7.68 (d, J=1.3 Hz, 1H), 7.64-7.62 (m, 4H), 7.46-7.43 (m, 6H), 7.24-7.22 (d, 1H), 7.14-7.12 (dd, J=8.6, 1.6 Hz, 1H), 3.48 (s, 2H), 2.63 (s, 2H), 1.08 (s, 9H), 0.88 (s, 6H).

Step 4. To a stirred mixture of HCOOH (66.3 g, 1.44 mol) in TEA (728 g, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to rt and 1-(3-bromopyridin-2-yl)ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄, and filtered. The filtrate was concentrated under reduced pressure and the residue was purified by silica gel column chromatography to give (1 S)-1-(3-bromopyridin-2-yl)ethanol (100 g, 74% yield) a an oil. LCMS (ESI): m/z [M+H] calc'd for C₇H₈BrNO 201.1; found 201.9.

Step 5. To a stirred mixture of (1 S)-1-(3-bromopyridin-2-yl)ethanol (100 g, 495 mmol) in DMF (1 L) at 0° C. was added NaH, 60% dispersion in oil (14.25 g, 594 mmol) in portions. The mixture was stirred at 0° C. for 1 h. Mel (140.5 g, 990 mmol) was added dropwise at 0° C. and the mixture was allowed to warm to rt and stirred for 2 h. The mixture was cooled to 0° C. and saturated NH₄Cl (5 L) was added. The mixture was extracted with EtOAc (3×1.5 L), dried over anhydrous Na₂SO₄ and filtered. The filtrate was concentrated under reduced pressure and the residue was purified by silica gel column chromatography to give 3-bromo-2-[(1 S)-1-methoxyethyl]pyridine (90 g, 75% yield) as an oil. LCMS (ESI): m/z [M+H] calc'd for C₈H₁₀BrNO 215.0; found 215.9.

Step 6. To a stirred mixture of 3-bromo-2-[(1S)-1-methoxyethyl]pyridine (90 g, 417 mmol) and Pd(dppf)Cl₂ (30.5 g, 41.7 mmol) in toluene (900 mL) at rt under an atmosphere of Ar was added bis(pinacolato)diboron (127 g, 500 mmol) and KOAc (81.8 g, 833 mmol) in portions. The mixture was heated to 100° C. and stirred for 3 h. The filtrate was concentrated under reduced pressure and the residue was purified by Al₂O₃ column chromatography to give 2-[(1 S)-1-methoxyethyl]-3-(4,4,5,5-tetramethyl-1,3,2-dioxaborolan-2-yl)pyridine (100 g, 63% yield) as a semi-solid. LCMS (ESI): m/z [M+H] calc'd for C₁₄H₂₂BNO₃ 263.2; found 264.1.

Step 7. To a stirred mixture of 5-bromo-3-[3-[(tert-butyldiphenylsilyl)oxy]-2,2-dimethylpropyl]-2-iodo-1H-indole (140 g, 217 mmol) and 2-[(1 S)-1-methoxyethyl]-3-(4,4,5,5-tetramethyl-1,3,2-dioxaborolan-2-yl)pyridine (100 g, 380 mmol) in 1,4-dioxane (1.4 L) at rt under an atmosphere of Ar was added K₂CO₃ (74.8 g, 541 mmol), Pd(dppf)Cl₂ (15.9 g, 21.7 mmol), and H₂O (280 mL) in portions. The mixture was heated to 85° C. and stirred for 4 h, then cooled, H₂O (5 L) added, and the mixture extracted with EtOAc (3×2 L). The combined organic layers were washed with brine (2×1 L), dried over anhydrous Na₂SO₄, and filtered. The filtrate was concentrated under reduced pressure and the residue was purified by silica gel column chromatography to give 5-bromo-3-[3-[(tert-butyldiphenylsilyl)oxy]-2,2-dimethylpropyl]-2-[2-[(1 S)-1-methoxyethyl]pyridin-3-yl]-1H-indole (71 g, 45% yield) as a solid. LCMS (ESI): m/z [M+H] calc'd for C₃₇H₄₃BrN₂O₂Si 654.2; found 655.1.

Step 8. To a stirred mixture of 5-bromo-3-[3-[(tert-butyldiphenylsilyl)oxy]-2,2-dimethylpropyl]-2-[2-[(1S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and Etl (33.8 g, 217 mmol) in portions. The mixture was warmed to rt and stirred for 16 h then H₂O (4 L) added and the mixture extracted with EtOAc (3×1.5 L). The combined organic layers were washed with brine (2×1 L), dried over anhydrous Na₂SO₄, and filtered. The filtrate was concentrated under reduced pressure and the residue was purified by silica gel column chromatography to give 5-bromo-3-[3-[(tert-butyldiphenylsilyl)oxy]-2,2-dimethylpropyl]-1-ethyl-2-[2-[(1S)-1-methoxyethyl]pyridin-3-yl]indole (66 g, 80% yield) as an oil. LCMS (ESI): m/z [M+H] calc'd for C₃₉H₄₇BrN₂O₂Si 682.3; found 683.3.

Step 9. To a stirred mixture of TBAF (172.6 g, 660 mmol) in THE (660 mL) at rt under an atmosphere of N₂ was added 5-bromo-3-[3-[(tert-butyldiphenylsilyl)oxy]-2,2-dimethylpropyl]-1-ethyl-2-[2-[(1 S)-1-methoxyethyl]pyridin-3-yl]indole (66 g, 97 mmol) in portions. The mixture was heated to 50° C. and stirred for 16 h, cooled, diluted with H₂O (5 L), and extracted with EtOAc (3×1.5 L). The combined organic layers were washed with brine (2×1 L), dried over anhydrous Na₂SO₄, and filtered. After filtration, the filtrate was concentrated under reduced pressure. The filtrate was concentrated under reduced pressure and the residue was purified by silica gel column chromatography to give 3-(5-bromo-1-ethyl-2-[2-[(1 S)-1-methoxyethyl]pyridin-3-yl]indol-3-yl)-2,2-dimethylpropan-1-ol (30 g, 62% yield) as a solid. LCMS (ESI): m/z [M+H] calc'd for C₂₃H₂₉BrN₂O₂ 444.1; found 445.1.

### Intermediate E1. Alternative Synthesis Through Fisher Indole Route

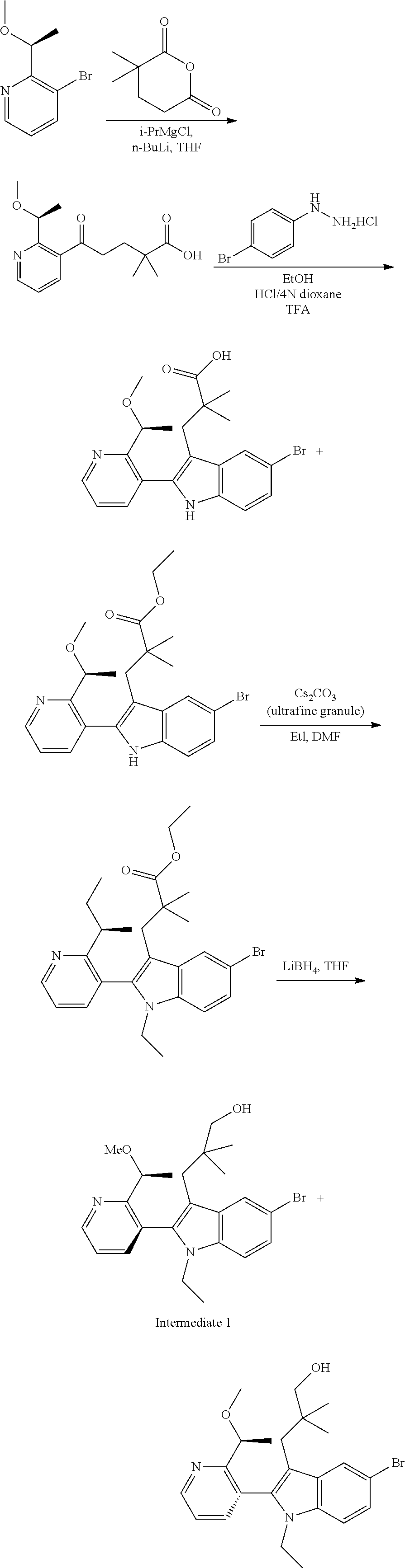

Step 1. To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at −10° C. then 3-bromo-2-[(1S)-1-methoxyethyl]pyridine (180 g, 833 mmol) in THE (0.5 L) added dropwise over 30 min at −10° C. The resulting mixture was warmed to −5° C. and stirred for 1 h, then 3,3-dimethyloxane-2,6-dione (118 g, 833 mmol) in THE (1.2 L) was added dropwise over 30 min at −5° C. The mixture was warmed to 0° C. and stirred for 1.5 h, then quenched with the addition of pre-cooled 4M HCl in 1,4-dioxane (0.6 L) at 0° C. to adjust pH ˜5. The mixture was diluted with ice-water (3 L) and extracted with EtOAc (3×2.5 L). The combined organic layers were dried over anhydrous Na₂SO₄, filtered, the filtrate was concentrated under reduced pressure, and the residue was purified by silica gel column chromatography to give 5-[2-[(1 S)-1-methoxyethyl]pyridin-3-yl]-2,2-dimethyl-5-oxopentanoic acid (87 g, 34% yield) as a solid. LCMS (ESI): m/z [M+H] calc'd for C₁₅H₂₁NO₄ 279.2; found 280.1.

Step 2. To a mixture of 5-[2-[(1 S)-1-methoxyethyl]pyridin-3-yl]-2,2-dimethyl-5-oxopentanoic acid (78 g, 279 mmol) in EtOH (0.78 L) at rt under an atmosphere of N₂ was added (4-bromophenyl)hydrazine HCl salt (68.7 g, 307 mmol) in portions. The mixture was heated to 85° C. and stirred for 2 h, cooled to rt, then 4M HCl in 1,4-dioxane (69.8 mL, 279 mmol) added dropwise. The mixture was heated to 85° C. and stirred for an additional 3 h, then concentrated under reduced pressure, and the residue was dissolved in TFA (0.78 L). The mixture was heated to 60° C. and stirred for 1.5 h, concentrated under reduced pressure, and the residue adjusted to pH ˜5 with saturated NaHCO₃, then extracted with EtOAc (3×1.5 L). The combined organic layers were dried over anhydrous Na₂SO₄, filtered, the filtrate concentrated under reduced pressure, and the residue was purified by silica gel column chromatography to give 3-(5-bromo-2-[2-[(1 S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate (78 g, crude). LCMS (ESI): m/z [M+H] calc'd for C₂₁H₂₃BrN₂O₃ 430.1 and C₂₃H₂₇BrN₂O₃ 458.1; found 431.1 and 459.1.

Step 3. To a mixture of 3-(5-bromo-2-[2-[(1 S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate (198 g, 459 mmol) in DMF (1.8 L) at 0° C. under an atmosphere of N₂ was added Cs₂CO₃ (449 g, 1.38 mol) in portions. Etl (215 g, 1.38 mmol) in DMF (200 mL) was then added dropwise at 0° C. The mixture was warmed to rt and stirred for 4 h then diluted with brine (5 L) and extracted with EtOAc (3×2.5 L). The combined organic layers were washed with brine (2×1.5 L), dried over anhydrous Na₂SO₄, and filtered. The filtrate was concentrated under reduced pressure and the residue was purified by silica gel column chromatography to give ethyl 3-(5-bromo-1-ethyl-2-[2-[(1 S)-1-methoxyethyl]pyridin-3-yl]indol-3-yl)-2,2-dimethylpropanoate (160 g, 57% yield) as a solid. LCMS (ESI): m/z [M+H] calc'd for C₂₅H₃₁BrN₂O₃ 486.2; found 487.2.

Step 4. To a mixture of ethyl 3-(5-bromo-1-ethyl-2-[2-[(1 S)-1-methoxyethyl]pyridin-3-yl]indol-3-yl)-2,2-dimethylpropanoate (160 g, 328 mmol) in THE (1.6 L) at 0° C. under an atmosphere of N₂ was added LiBH₄ (28.6 g, 1.3 mol). The mixture was heated to 60° C. for 16 h, cooled, and quenched with pre-cooled (0° C.) aqueous NH₄Cl (5 L). The mixture was extracted with EtOAc (3×2 L) and the combined organic layers were washed with brine (2×1 L), dried over anhydrous Na₂SO₄, and filtered. The filtrate was concentrated under reduced pressure and the residue was purified by silica gel column chromatography to give to two atropisomers (as single atropisomers) of 3-(5-bromo-1-ethyl-2-(2-((S)-1-methoxyethyl)pyridin-3-yl)-1H-indol-3-yl)-2,2-dimethylpropan-1-ol (60 g, 38% yield) and (40 g, 26% yield) both as solids. LCMS (ESI): m/z [M+H] calc'd for C₂₃H₂₉BrN₂O₂ 444.1; found 445.2.

### Intermediate E2. Synthesis of Tert-Butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

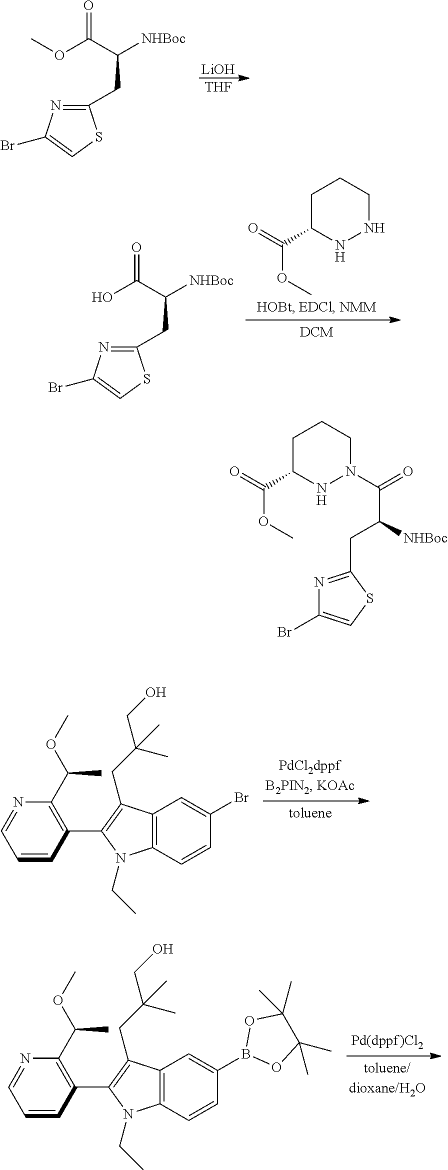

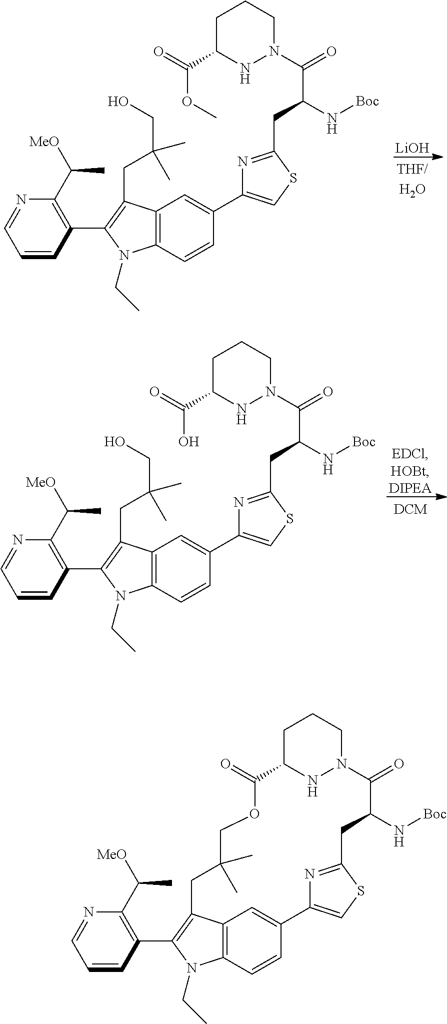

Step 1. To a solution of methyl (2S)-3-(4-bromo-1,3-thiazol-2-yl)-2-[(tert-butoxycarbonyl)amino]propanoate (110 g, 301.2 mmol) in THE (500 mL) and H₂O (200 mL) at room temperature was added LiOH (21.64 g, 903.6 mmol). The resulting solution was stirred for 1 h and was then concentrated under reduced pressure. The resulting residue was adjusted to pH 6 with 1 M HCl and then extracted with DCM (3×500 mL). The combined organic layers were, dried over Na₂SO₄, filtered, and concentrated under reduced pressure to afford the desired product (108 g, crude). LCMS (ESI) m/z: [M+H] calcd for C₁₁H₁₅BrN₂O₄S: 351.00; found 351.0.

Step 2. To a solution of (S)-3-(4-bromothiazol-2-yl)-2-((tert-butoxycarbonyl)amino)propanoic acid (70 g, 199.3 mmol) in DCM (500 mL) at 0° C. was added methyl (3S)-1,2-diazinane-3-carboxylate bis(trifluoroacetic acid) salt (111.28 g, 298.96 mmol), NMM (219.12 mL. 1993.0 mmol), EDCI (76.41 g, 398.6 mmol) and HOBt (5.39 g, 39.89 mmol). The resulting solution was warmed to room temperature and stirred for 1 h. The reaction was then quenched with H₂O (500 mL) and was extracted with EtOAc (3×500 mL). The combined organic layers were dried over Na₂SO₄, filtered, and concentrated under reduced pressured. The residue was purified by silica gel chromatography (0→50% EtOAc/pet. ether) to afford the desired product (88.1 g, 92.6% yield). LCMS (ESI) m/z: [M+H] calcd for C₁₇H₂₅BrN₄O₅S: 477.08; found 477.1.

Step 3. To a solution of 3-(5-bromo-1-ethyl-2-(2-((S)-1-methoxyethyl)pyridin-3-yl)-1H-indol-3-yl)-2,2-dimethylpropan-1-ol (60 g, 134.7 mmol) in toluene (500 mL) at room temperature was added bis(pinacolato)diboron (51.31 g, 202.1 mmol), Pd(dppf)Cl₂ (9.86 g, 13.48 mmol) and KOAc (26.44 g, 269.4 mmol). Then reaction mixture was then heated to 90° C. and stirred for 2 h. The reaction solution was then cooled to room temperature and concentrated under reduced pressure. Purification by silica gel chromatography (0→50% EtOAc/pet. ether) afforded the desired product (60.6 g, 94.0% yield). LCMS (ESI) m/z: [M+H] calcd for C₂₉H₄₁BN₂O₄: 493.32; found 493.3.

Step 4. To a solution of (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (30 g, 60.9 mmol) in toluene (600 mL), dioxane (200 mL), and H₂O (200 mL) at room temperature was added methyl (S)-1-((S)-3-(4-bromothiazol-2-yl)-2-((tert-butoxycarbonyl)amino)propanoyl)hexahydropyridazine-3-carboxylate (43.62 g, 91.4 mmol), K₃PO₄ (32.23 g, 152.3 mmol) and Pd(dppf)Cl₂ (8.91 g, 12.18 mmol). The resulting solution was heated to 70° C. and stirred overnight. The reaction mixture was then cooled to room temperature and was quenched with H₂O (200 mL). The resulting mixture was extracted with EtOAc (3×1000 mL) and the combined organic layers were dried over Na₂SO₄, filtered, and concentrated under reduced pressure. The residue was purified by silica gel chromatography (0→90% EtOAc/pet. ether) to afford the desired product (39.7 g, 85.4% yield). LCMS (ESI) m/z: [M+H] calcd for C₄₀H₅₄N₆O₇S: 763.39; found 763.3.

Step 5. To a solution of methyl (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylate (39.7 g, 52.0 mmol) in THE (400 mL) and H₂O (100 mL) at room temperature was added LiOH⋅H₂O (3.74 g, 156.2 mmol). The resulting mixture was stirred for 1.5 h and was then concentrated under reduced pressure. The residue was acidified to pH 6 with 1 M HCl and extracted with DCM (3×1000 mL). The combined organic layers were dried over Na₂SO₄, filtered, and concentrated under reduced pressure to afford the desired product (37.9 g, crude). LCMS (ESI) m/z: [M+H] calcd for C₃₉H₅₂N₆O₇S: 749.37; found 749.4.

Step 6. To a solution of (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylic acid (37.9 g, 50.6 mmol), HOBt (34.19 g, 253.0 mmol) and DIPEA (264.4 mL, 1518 mmol) in DCM (4 L) at 0° C. was added EDCI (271.63 g, 1416.9 mmol). The resulting mixture was warmed to room temperature and stirred overnight. The reaction mixture was then quenched with H₂O and washed with 1 M HCl (4×1 L). The organic layer was separated and concentrated under reduced pressure. The residue was purified by silica gel chromatography (0→70% EtOAc/pet. ether) to afford the desired product (30 g, 81.1% yield). LCMS (ESI) m/z: [M+H] calcd for C₃₉H₅₀N₆O₆S: 731.36; found 731.3.

### Intermediate E3. Synthesis of (S)-3-bromo-5-iodo-2-(1-methoxyethyl) pyridine

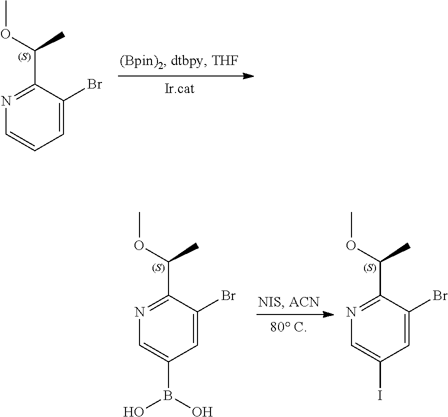

Step 1. To a stirred solution of 3-bromo-2-[(1S)-1-methoxyethyl]pyridine (80.00 g, 370.24 mmol, 1.00 equiv) and bis(pinacolato)diboron (141.03 g, 555.3 mmol, 1.50 equiv) in THE (320 mL) was added dtbpy (14.91 g, 55.5 mmol) and Chloro(1,5-cyclooctadiene)iridium(I) dimer (7.46 g, 11.1 mmol) under argon atmosphere. The resulting mixture was stirred for 16 h at 75° C. under argon atmosphere. The mixture was concentrated under reduced pressure. The resulting mixture was dissolved in EtOAc (200 mL) and the mixture was adjusted to pH 10 with Na₂CO₃ (40 g) and NaOH (10 g) (mass 4:1) in water (600 mL). The aqueous layer was extracted with EtOAc (800 mL). The aqueous phase was acidified to pH=6 with HCl (6 NV) to precipitate the desired solid to afford 5-bromo-6-[(1S)-1-methoxyethyl]pyridin-3-ylboronic acid (50 g, 52.0% yield) as a light-yellow solid. LCMS (ESI): m/z [M+H] calc'd for C₈H₁₁BBrNO₃ 259.0; found 260.0.

Step 2. To a stirred solution of 5-bromo-6-[(1S)-1-methoxyethyl]pyridin-3-ylboronic acid (23.00 g, 88.5 mmol) in ACN (230 mL) were added NIS (49.78 g, 221.2 mmol) at room temperature under argon atmosphere. The resulting mixture was stirred for overnight at 80° C. under argon atmosphere. The resulting mixture was concentrated under reduced pressure. The resulting mixture was dissolved in DCM (2.1 L) and washed with Na₂S₂O₃ (3×500 mL). The organic layer was dried over anhydrous Na₂SO₄. After filtration, the filtrate was concentrated under reduced pressure. The residue was purified by silica gel column chromatography to afford (S)-3-bromo-5-iodo-2-(1-methoxyethyl)pyridine (20 g, 66.0% yield).

LCMS (ESI): m/z [M+H] calc'd for C₈H₉BrINO 340.9; found 341.7.

### Intermediate E4. Synthesis of Tert-Butyl ((6³S,4S,Z)-11-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1H-8-oxa-2(4,2)-thiazola-1(5,3)-indola-6(1,3)-pyridazinacycloundecaphane-4-yl)carbamate

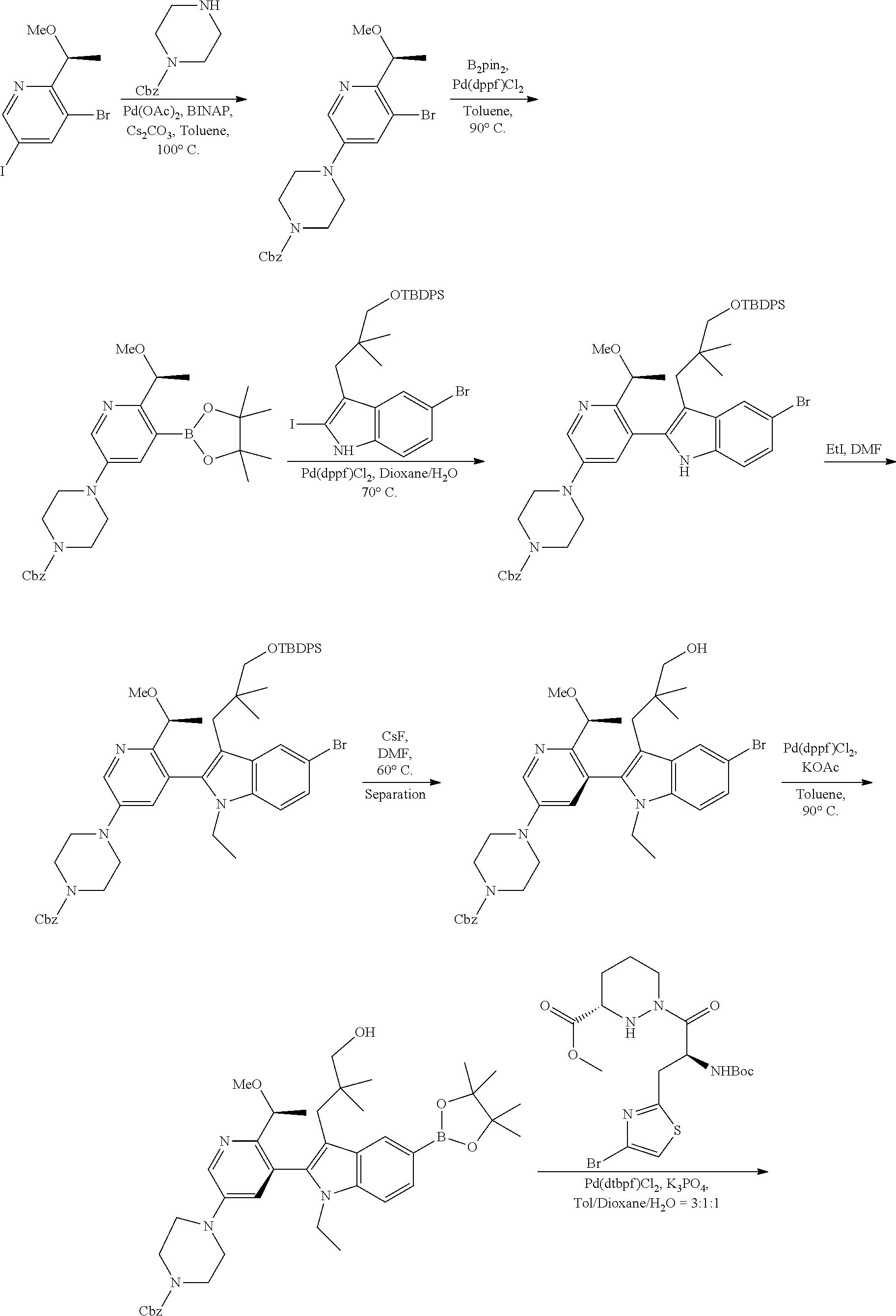

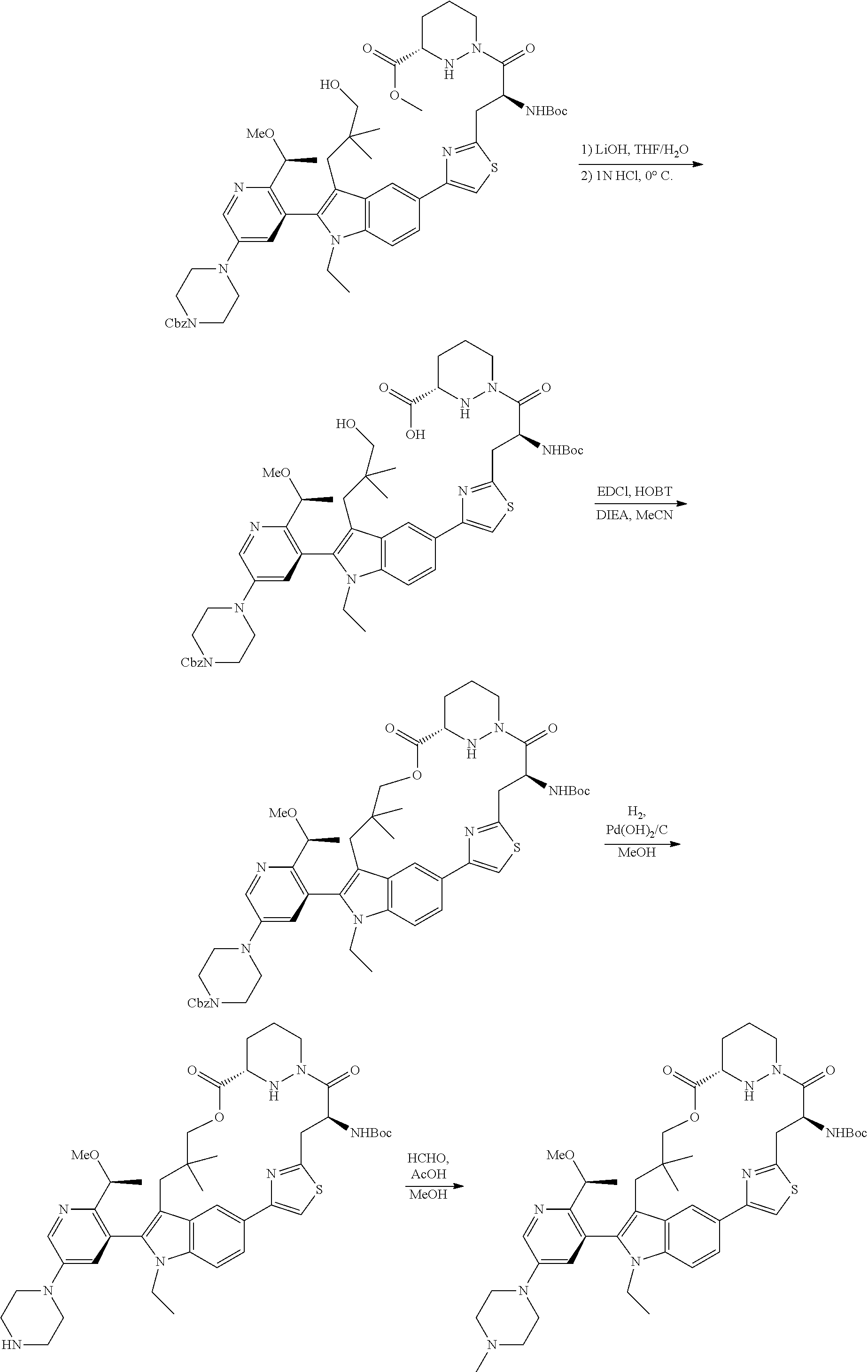

Step 1. Into a 3L 3-necked round-bottom flask purged and maintained with an inert atmosphere of argon, was placed 3-bromo-5-iodo-2-[(1 S)-1-methoxyethyl]pyridine (147 g, 429.8 mmol) benzyl piperazine-1-carboxylate (94.69 g, 429.8 mmol), Pd(OAc)₂ (4.83 g, 21.4 mmol), BINAP (5.35 g, 8.6 mmol), Cs₂CO₃ (350.14 g, 1074.6 mmol), toluene (1 L). The resulting solution was stirred for overnight at 100° C. in an oil bath. The reaction mixture was cooled to 25° C. after reaction completed. The resulting mixture was concentrated under reduced pressure. The residue was applied onto a silica gel column with ethyl acetate/hexane (1:1). Removal of solvent under reduced pressure gave benzyl (S)-4-(5-bromo-6-(1-methoxyethyl)pyridin-3-yl)piperazine-1-carboxylate (135 g, 65.1% yield) as a dark yellow solid. LCMS (ESI): m/z [M+H] calc'd for C₂₀H₂₄BrN₃O₃ 433.1; found 434.1.

Step 2. Into a 3-L 3-necked round-bottom flask purged and maintained with an inert atmosphere of argon, was placed benzyl 4-[5-bromo-6-[(1 S)-1-methoxyethyl]pyridin-3-yl]piperazine-1-carboxylate (135 g, 310.8 mmol), bis(pinacolato)diboron (86.82 g, 341.9 mmol), Pd(dppf)Cl₂ (22.74 g, 31.0 mmol), KOAc (76.26 g, 777.5 mmol), Toluene (1 L). The resulting solution was stirred for 2 days at 90° C. in an oil bath. The reaction mixture was cooled to 25° C. The resulting mixture was concentrated under vacuum. The residue was applied onto a neutral alumina column with ethyl acetate/hexane (1:3). Removal of solvent under reduced pressure gave benzyl (S)-4-(6-(1-methoxyethyl)-5-(4,4,5,5-tetramethyl-1,3,2-dioxaborolan-2-yl)pyridin-3-yl)piperazine-1-carboxylate (167 g, crude) as a dark yellow solid. LCMS (ESI): m/z [M+H] calc'd for C₂₆H₃₆BN₃O₅ 481.3; found 482.1.

Step 3. Into a 3-L 3-necked round-bottom flask purged and maintained with an inert atmosphere of argon, was placed (S)-4-(6-(1-methoxyethyl)-5-(4,4,5,5-tetramethyl-1,3,2-dioxaborolan-2-yl)pyridin-3-yl)piperazine-1-carboxylate (167 g, 346.9 mmol), 5-bromo-3-[3-[(tert-butyldiphenylsilyl)oxy]-2,2-dimethylpropyl]-2-iodo-1H-indole (224.27 g, 346.9 mmol), Pd(dppf)Cl₂ (25.38 g, 34.6 mmol), dioxane (600 mL), H₂O (200 mL), K₃PO₄ (184.09 g, 867.2 mmol), Toluene (200 mL). The resulting solution was stirred for overnight at 70° C. in an oil bath. The reaction mixture was cooled to 25° C. after reaction completed. The resulting mixture was concentrated under vacuum. The residue was applied onto a silica gel column with ethyl acetate/hexane (1:1). Removal of solvent under reduced pressure gave benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl)piperazine-1-carboxylate (146 g, 48.1% yield) as a yellow solid. LCMS (ESI): m/z [M+H] calc'd for C₄₉H₅₇BrN₄O₄Si 872.3; found 873.3.

Step 4. To a stirred mixture of benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl)piperazine-1-carboxylate (146 g, 167.0 mmol) and Cs₂CO₃ (163.28 g, 501.1 mmol) in DMF (1200 mL) was added C₂H₅₁ (52.11 g, 334.0 mmol) in portions at 0° C. under N₂ atmosphere. The final reaction mixture was stirred at 25° C. for 12 h. Desired product could be detected by LCMS. The resulting mixture was diluted with EA (1 L) and washed with brine (3×1.5L). The organic layers were dried over anhydrous Na₂SO₄. After filtration, the filtrate was concentrated under reduced pressure to give benzyl (S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl)piperazine-1-carboxylate (143 g, crude) as a yellow solid that was used directly for next step without further purification. LCMS (ESI): m/z [M+H] calc'd for C₅₁H₆₁BrN₄O₄Si 900.4; found 901.4.

Step 5. To a stirred mixture of benzyl benzyl (S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl)piperazine-1-carboxylate (143 g, 158.5 mmol) in DMF (1250 mL) was added CsF (72.24 g, 475.5 mmol). Then the reaction mixture was stirred at 60° C. for 2 days under N₂ atmosphere. Desired product could be detected by LCMS. The resulting mixture was diluted with EA (1 L) and washed with brine (3×1 L). Then the organic phase was concentrated under reduced pressure. The residue was purified by silica gel column chromatography, eluted with PE/EA (⅓) to afford two atropisomers of benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate A (38 g, 36% yield, RT=1.677 min in 3 min LCMS(0.1% FA)) and B (34 g, 34% yield, RT=1.578 min in 3 min LCMS(0.1% FA)) both as yellow solid. LCMS (ESI): m/z [M+H] calc'd for C₃₅H₄₃BrN₄O₄ 663.2; found 662.2.

Step 6. Into a 500-mL 3-necked round-bottom flask purged and maintained with an inert atmosphere of nitrogen, was placed benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate A (14 g, 21.1 mmol), bis(pinacolato)diboron (5.89 g, 23.21 mmol), Pd(dppf)Cl₂ (1.54 g, 2.1 mmol), KOAc (5.18 g, 52.7 mmol), Toluene (150 mL). The resulting solution was stirred for 5 h at 90° C. in an oil bath. The reaction mixture was cooled to 25° C. The resulting mixture was concentrated under vacuum. The residue was purified by silica gel column chromatography, eluted with PE/EA (⅓) to give benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate (12 g, 76.0% yield) as a yellow solid. LCMS (ESI): m/z [M+H] calc'd for C₄₁H₅₅BN₄O₆ 710.4; found 711.3.

Step 7. Into a 250-mL round-bottom flask purged and maintained with an inert atmosphere of argon, was placed benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate (10.8 g, 15.2 mmol), methyl (3S)-1-[(2S)-3-(4-bromo-1,3-thiazol-2-yl)-2-[(tert-butoxycarbonyl)amino]propanoyl]-1,2-diazinane-3-carboxylate (7.98 g, 16.7 mmol), Pd(dtbpf)Cl₂ (0.99 g, 1.52 mmol), K₃PO₄ (8.06 g, 37.9 mmol), Toluene (60 mL), dioxane (20 mL), H₂O (20 mL). The resulting solution was stirred for 3 h at 70° C. in an oil bath. The reaction mixture was cooled to 25° C. The resulting solution was extracted with EtOAc (2×50 mL) and concentrated under reduced pressure. The residue was applied onto a silica gel column with ethyl acetate/hexane (10:1). Removal of solvent to give methyl (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylate (8 g, 50.9% yield) as a yellow solid. LCMS (ESI): m/z [M+H] calc'd for C₅₂H₆₈N₈O₉S 980.5; found 980.9.

Step 8. To a stirred mixture of methyl (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylate (12 g, 12.23 mmol) in THE (100 mL)/H₂O (100 mL) was added LiOH (2.45 g, 61.1 mmol) under N₂ atmosphere and the resulting mixture was stirred for 2 h at 25° C. Desired product could be detected by LCMS. THE was concentrated under reduced pressure. The pH of aqueous phase was acidified to 5 with HCL (1N) at 0° C. The aqueous layer was extracted with DCM (3×100 ml). The organic phase was concentrated under reduced pressure to give (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylic acid (10 g, 84.5% yield) as a light yellow solid. LCMS (ESI): m/z [M+H] calc'd for C₅₁H₆₆N₈O₉S 966.5; found 967.0.

Step 9. Into a 3-L round-bottom flask purged and maintained with an inert atmosphere of nitrogen, was placed (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylic acid (18 g, 18.61 mmol), ACN (1.8 L), DIEA (96.21 g, 744.4 mmol), EDCI (107.03 g, 558.3 mmol), HOBT (25.15 g, 186.1 mmol). The resulting solution was stirred for overnight at 25° C. The resulting mixture was concentrated under vacuum after reaction completed. The resulting solution was diluted with DCM (1 L). The resulting mixture was washed with HCl (3×1 L, 1N aqueous). The resulting mixture was washed with water (3×1 L). Then the organic layer was concentrated, the residue was applied onto a silica gel column with ethyl acetate/hexane (1:1). Removal of solvent under reduced pressure gave benzyl 4-(5-((6³S,4S,Z)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl)piperazine-1-carboxylate (10.4 g, 54.8% yiels) as a light yellow solid. LCMS (ESI): m/z [M+H] calc'd for C₅₁H₆₄N₈O₈S 948.5; found 949.3.

Step 10. Into a 250-mL round-bottom flask purged and maintained with an inert atmosphere of nitrogen, was placed benzyl 4-(5-((6³S,4S,Z)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl)piperazine-1-carboxylate (10.40 g, 10.9 mmol), Pd(OH)₂/C (5 g, 46.9 mmol), MeOH (100 mL). The resulting solution was stirred for 3 h at 25° C. under 2 atm H₂ atmosphere. The solids were filtered out and the filter cake was washed with MeOH (3×100 mL). Then combined organic phase was concentrated under reduced pressure to give tert-butyl ((6³S,4S,Z)-11-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (8.5 g, 90.4% yield) as a light yellow solid. LCMS (ESI): m/z [M+H] calc'd for C₄₃H₅₈N806S 814.4; found 815.3.

Step 11. Into a 1000-mL round-bottom flask purged and maintained with an inert atmosphere of nitrogen, was placed tert-butyl ((6³S,4S,Z)-11-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)carbamate (8.5 g, 10.4 mmol), MeOH (100 mL), AcOH (1.88 g, 31.2 mmol) and stirred for 15 mins. Then HCHO (1.88 g, 23.15 mmol, 37% aqueous solution) and NaBH₃CN (788 mg, 12.5 mmol) was added at 25° C. The resulting solution was stirred for 3 h at 25° C. The resulting mixture was quenched with 100 mL water and concentrated under reduced pressure to remove MeOH. The resulting solution was diluted with 300 mL of DCM. The resulting mixture was washed with water (3×100 mL). Removal of solvent gave tert-butyl ((6³S,4S,Z)-1¹-ethyl-12-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (8.2 g, 90.1% yield) as a yellow solid. LCMS (ESI): m/z [M+H] calc'd for C₄₄H₆₀N₈O₆S 828.4; found 829.3.

### Intermediate E5. Synthesis of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

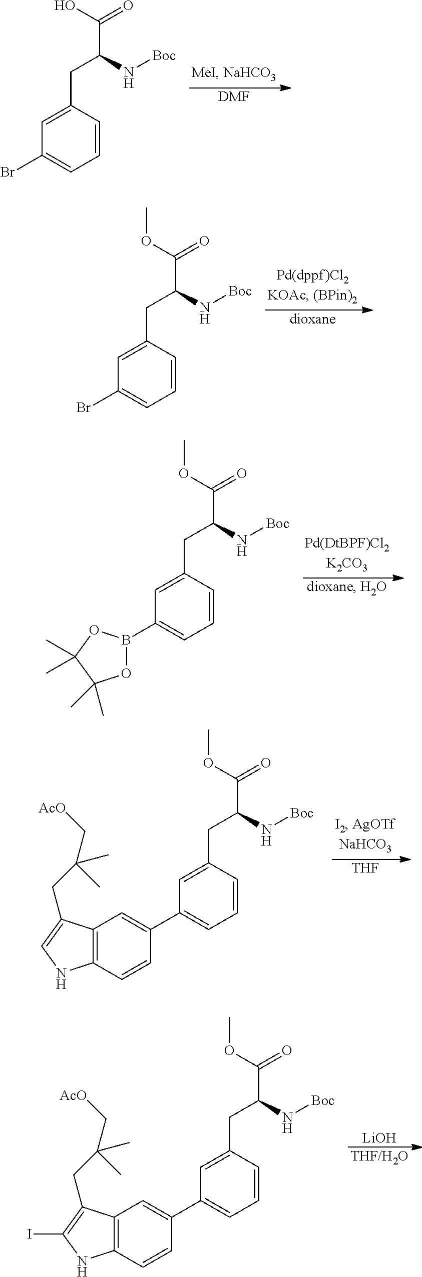

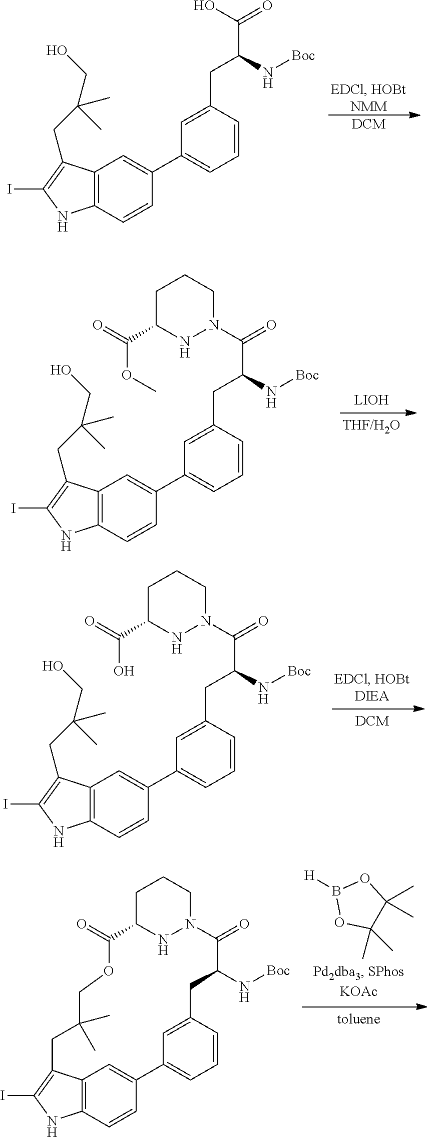

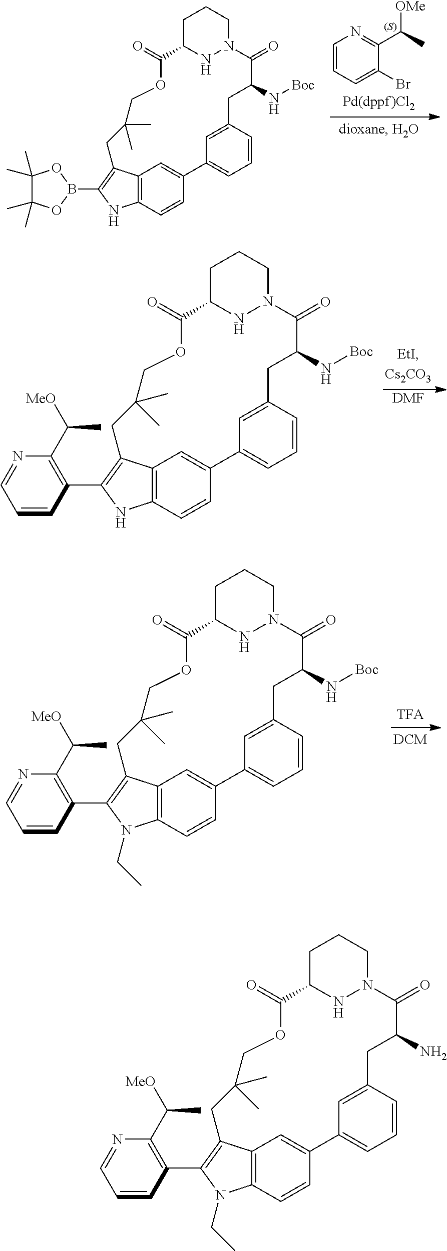

Step 1. To a solution of (2S)-3-(3-bromophenyl)-2-[(tert-butoxycarbonyl)amino]propanoic acid (100 g, 290 mmol) in DMF (1 L) at room temperature was added NaHCO₃ (48.8 g, 581.1 mmol) and Mel (61.9 g, 435.8 mmol). The reaction mixture was stirred for 16 h and was then quenched with H₂O (1 L) and extracted with EtOAc (3×1 L). The combined organic layers were washed with brine (3×500 mL), dried over Na₂SO₄, filtered, and concentrated under reduced pressure. The residue was purified by silica gel column chromatography (13% EtOAc/pet. ether) to give the final product (109 g, crude). LCMS (ESI) m/z [M+Na] calcd for C₁₅H₂₀BrNO₄ 380.05; found: 380.0.

Step 2. To a stirred solution of methyl (2S)-3-(3-bromophenyl)-2-[(tert-butoxycarbonyl)amino]propanoate (108 g, 301.5 mmol) and bis(pinacolato)diboron (99.53 g, 391.93 mmol) in dioxane (3.2 L) was added KOAc (73.97 g, 753.70 mmol) and Pd(dppf)Cl₂ (22.06 g, 30.15 mmol). The reaction mixture was heated to 90° C. for 3 h and was then cooled to room temperature and extracted with EtOAc (2×3 L). The combined organic layers were washed with brine (3×800 mL), dried over Na₂SO₄, filtered, and concentrated under reduced pressure. The residue was purified by silica gel column chromatography (5% EtOAc/pet. ether) to afford the product (96 g, 78.6% yield). LCMS (ESI) m/z [M+Na] calcd for C₂₁H₃₂BNO₆ 428.22; found: 428.1.

Step 3. To a mixture of methyl (2S)-2-[(tert-butoxycarbonyl)amino]-3-[3-(4,4,5,5-tetramethyl-1,3,2-dioxaborolan-2-yl)phenyl]propanoate (94 g, 231.9 mmol) and 3-(5-bromo-1H-indol-3-yl)-2,2-dimethylpropyl acetate (75.19 g, 231.93 mmol) in dioxane (1.5 L) and H₂O (300 mL) was added K₂CO₃ (64.11 g, 463.85 mmol) and Pd(DtBPF)Cl₂ (15.12 g, 23.19 mmol). The reaction mixture was heated to 70° C. and stirred for 4 h. The reaction mixture was extracted with EtOAc (2×2 L) and the combined organic layers were washed with brine (3×600 mL), dried over Na₂SO₄, filtered, and concentrated under reduced pressure. The residue was purified by silica gel column chromatography (20% EtOAc/pet. ether) to give the product (130 g, crude). LCMS (ESI) m/z [M+H] calcd for C₃₀H₃₈N₂O₆ 523.28; found: 523.1.

Step 4. To a solution of methyl (2S)-3-(3-[3-[3-(acetyloxy)-2,2-dimethylpropyl]-1H-indol-5-yl]phenyl)-2-[(tert-butoxycarbonyl)amino]propanoate (95.0 g, 181.8 mmol) and iodine (36.91 g, 145.41 mmol) in THE (1 L) at −10° C. was added AgOTf (70.0 g, 272.7 mmol) and NaHCO₃(22.9 g, 272.65 mmol). The reaction mixture was stirred for 30 min and was then quenched by the addition of sat. aq. Na₂S₂O₃ (100 mL) at 0° C. The resulting mixture was extracted with EtOAc (3×1 L) and the combined organic layers were washed with brine (3×500 mL), dried over Na₂SO₄, filtered, and concentrated under reduced pressure. The residue was purified by silica gel column chromatography (50% EtOAc/pet. ether) to give methyl (S)-3-(3-(3-(3-acetoxy-2,2-dimethylpropyl)-2-iodo-1H-indol-5-yl)phenyl)-2-((tert-butoxycarbonyl)amino)propanoate (49.3 g, 41.8% yield). LCMS (ESI) m/z [M+H] calcd for C₃₀H₃₇IN₂O₆: 649.18; found: 649.1.

Step 5. To a solution of methyl (2S)-3-(3-[3-[3-(acetyloxy)-2,2-dimethylpropyl]-2-iodo-1H-indol-5-yl]phenyl)-2-[(tert-butoxycarbonyl)amino]propanoate (60 g, 92.5 mmol) in THE (600 mL) was added a solution of LiOH⋅H₂O (19.41 g, 462.5 mmol) in H₂O (460 mL). The resulting solution was stirred overnight and then the pH was adjusted to 6 with HCl (1 M). The resulting solution was extracted with EtOAc (2×500 mL) and the combined organic layers was washed with brine (2×500 mL), dried over Na₂SO₄, filtered, and concentrated under reduced pressure to give the product (45 g, 82.1% yield). LCMS (ESI) m/z [M+Na] calcd for C₂₇H₃₃IN₂O₆ 615.13; found: 615.1.

Step 6. To a solution of (2S)-2-[(tert-butoxycarbonyl)amino]-3-[3-[3-(3-hydroxy-2,2-dimethylpropyl)-2-iodo-1H-indol-5-yl]phenyl]propanoic acid (30 g, 50.6 mmol) and methyl (3S)-1,2-diazinane-3-carboxylate (10.9 g, 75.9 mmol) in DCM (400 mL) was added NMM (40.97 g, 405.08 mmol), HOBt (2.05 g, 15.19 mmol), and EDCI (19.41 g, 101.27 mmol). The reaction mixture was stirred overnight and then the mixture was washed with sat. aq. NH₄Cl (2×200 mL) and brine (2×200 mL), and the mixture was dried over Na₂SO₄, filtered, and concentrated under reduced pressure to give the product (14 g, 38.5% yield). LCMS (ESI) m/z [M+H] calcd for C₃₃H₄₃IN₄O₆ 718.23; found: 719.4.

Step 7. To a solution of methyl (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylate (92 g, 128.0 mmol) in THE (920 mL) at 0° C. was added a solution of LiOH⋅H₂O (26.86 g, 640.10 mmol) in H₂O (640 mL). The reaction mixture was stirred for 2 h and was then concentrated under reduced pressure to give the product (90 g, crude). LCMS (ESI) m/z [M+H] calcd for C₃₂H₄₁IN₄O₆ 705.22; found: 705.1.

Step 8. To a solution of (3S)-1-[(2S)-2-[(tert-butoxycarbonyl)amino]-3-[3-[3-(3-hydroxy-2,2-dimethylpropyl)-2-iodo-1H-indol-5-yl]phenyl]propanoyl]-1,2-diazinane-3-carboxylic acid (90 g, 127.73 mmol) in DCM (10 L) at 0° C. was added HOBt (34.52 g, 255.46 mmol), DIPEA (330.17 g, 2554.62 mmol) and EDCI (367.29 g, 1915.96 mmol). The reaction mixture was stirred for 16 h and was then concentrated under reduced pressure. The mixture was extracted with DCM (2×2 L) and the combined organic layers were washed with brine (3×1 L), dried over Na₂SO₄, filtered, and concentrated under reduced pressure. The residue was purified by silica gel column chromatography (50% EtOAc/pet. ether) to give the product (70 g, 79.8% yield). LCMS (ESI) m/z [M+H] calcd for C₃₂H₃₉IN₄O₅ 687.21; found: 687.1.

Step 9. A 1 L round-bottom flask was charged with tert-butyl ((6³S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (22.0 g, 32.042 mmol), toluene (300.0 mL), Pd₂(dba)₃ (3.52 g, 3.845 mmol), S-Phos (3.95 g, 9.613 mmol), and KOAc (9.43 g, 96.127 mmol) at room temperature. To the mixture was added 4,4,5,5-tetramethyl-1,3,2-dioxaborolane (26.66 g, 208.275 mmol) dropwise with stirring at room temperature. The resulting solution was stirred for 3 h at 60° C. The resulting mixture was filtered, and the filter cake was washed with EtOAc. The filtrate was concentrated under reduced pressure and the remaining residue was purified by silica gel column chromatography to afford the product (22 g, 90% yield) as a solid. LCMS (ESI) m/z [M+H] calcd for C₃₈H₅₁BN₄O₇ 687.3; found: 687.4.

Step 10. A mixture of tert-butyl ((6³S,4S)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (2.0 g, 2.8 mmol), 3-bromo-2-[(1 S)-1-methoxyethyl]pyridine (0.60 g, 2.8 mmol), Pd(dppf)Cl₂ (0.39 g, 0.5 mmol), and K₃PO₄ (1.2 g, 6.0 mmol) in dioxane (50 mL) and H₂O (10 mL) under an atmosphere of N₂ was heated to 70° C. and stirred for 2 h. The mixture was diluted with H₂O (50 mL) and extracted with EtOAc (3×50 mL). The combined organic layers were washed with brine (3×50 mL), dried over anhydrous Na₂SO₄, and filtered. The filtrate was concentrated under reduced pressure and the residue was purified by silica gel column chromatography to afford the product (1.5 g, 74% yield) as a solid. LCMS (ESI) m/z [M+H] calcd for C₄₀H₄₉N₅O₆ 695.4; found: 696.5.

Step 11. To a solution of tert-butyl ((6³S,4S)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl) carbamate (20 g, 28.7 mmol) and Cs₂CO₃ (18.7 g, 57.5 mmol) in DMF (150 mL) at 0° C. was added a solution of Etl (13.45 g, 86.22 mmol) in DMF (50 mL). The resulting mixture was stirred overnight at 35° C. and then diluted with H₂O (500 mL). The mixture was extracted with EtOAc (2×300 mL) and the combined organic layers were washed with brine (3×100 mL), dried over Na₂SO₄, filtered, and concentrated under reduced pressure. The residue was purified by silica gel column chromatography to afford the product (4.23 g, 18.8% yield) and the atropisomer (5.78 g, 25.7% yield) as solids. LCMS (ESI) m/z [M+H] calcd for C₄₂H₅₃N₅O₆ 724.4; found: 724.6.

Step 12. A mixture of tert-butyl ((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.3 g, 1.7 mmol) in TFA (10 mL) and DCM (20 mL) was stirred at 0° C. for 2 h. The mixture was concentrated under reduced pressure to afford the product (1.30 g, crude) as a solid. LCMS (ESI) m/z [M+H] calcd for C₃₇H₄₅N₅O₄ 623.3; found: 624.4.

### Intermediate E6: Synthesis of (S)-3-(5-bromo-1-ethyl-2-(5-iodo-2-(1-methoxyethyl)pyridin-3-yl)-1H-indol-3-yl)-2,2-dimethylpropyl acetate

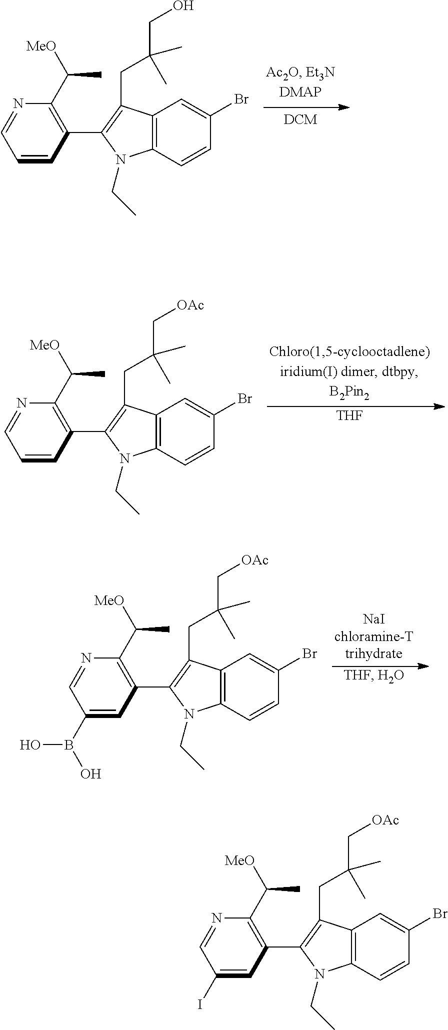

Step 1. To a stirred solution of (S)-3-(5-bromo-1-ethyl-2-(2-(1-methoxyethyl)pyridin-3-yl)-1H-indol-3-yl)-2,2-dimethylpropan-1-ol (100 g, 224.517 mmol) and Et₃N (45.44 g, 449.034 mmol) in DCM (1 L) was added DMAP (2.74 g, 22.452 mmol) and Ac₂O (27.50 g, 269.420 mmol) in portions at 0° C. under an argon atmosphere. The resulting mixture was stirred for 3 h at room temperature. The resulting mixture was concentrated under reduced pressure then diluted with EtOAc (1000 mL). The resulting mixture was washed with 1 M HCl (500 mL) then washed with sat. NaHCO₃ (500 mL) and brine (500 mL) dried over anhydrous Na₂SO₄. After filtration, the filtrate was concentrated under reduced pressure. The residue was purified by trituration with pet. ether (500 mL) to afford the product (93.3 g, 85% yield) as a white solid.

LCMS (ESI) m/z [M+H] calcd for C₂₅H₃₁BrN₂O₃: 487.16; found: 489.2

Step 2. To a stirred solution of (S)-3-(5-bromo-1-ethyl-2-(2-(1-methoxyethyl)pyridin-3-yl)-1H-indol-3-yl)-2,2-dimethylpropyl acetate (93.3 g, 191.409 mmol) and B₂PIN₂ (72.91 g, 287.113 mmol) in THE (370 mL) was added dtbpy (7.71 g, 28.711 mmol) and chloro(1,5-cyclooctadiene)iridium(I) dimer (6.43 g, 9.570 mmol) in portions at room temperature under an argon atmosphere. The resulting mixture was stirred overnight at 75° C. The resulting mixture was concentrated under reduced pressure to afford the product (190 g, crude) as an oil. LCMS(ESI) m/z [M+H]; calcd for C₂₅H₃₂BBrN₂O₅: 531.17; found: 533.3 Step 3. To a stirred solution of (S)-(5-(3-(3-acetoxy-2,2-dimethylpropyl)-5-bromo-1-ethyl-1H-indol-2-yl)-6-(1-methoxyethyl)pyridin-3-yl)boronic acid (110 g, 207.059 mmol) and chloramine-T trihydrate (349.96 g, 1242.354 mmol) in THE (550 mL) was added a solution of NaI (186.22 g, 1242.354 mmol) in H₂O (225 mL) in portions at 0° C. under an air atmosphere. The resulting mixture was stirred overnight at 50° C. under an argon atmosphere. The resulting mixture was concentrated under reduced pressure then washed with CHCl₃ (500 mL). The resulting mixture was filtered, the filter cake was washed with CHCl₃ (3 35×250 mL). The filtrate was extracted with CHCl₃ (3×500 mL). The combined organic layers were washed with Na₂S₂O₃ (500 mL), washed with brine (2×200 mL) dried over anhydrous Na₂SO₄. After filtration, the filtrate was concentrated under reduced pressure. The residue was purified by silica gel column

### Intermediate E7: Synthesis of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate

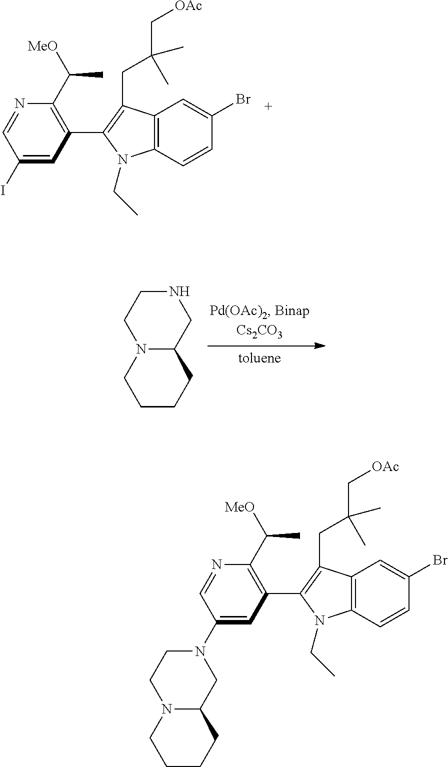

To a stirred solution of 3-(5-bromo-1-ethyl-2-{5-iodo-2-[(1 S)-1-methoxyethyl]pyridin-3-yl}indol-3-yl)-2,2-dimethylpropyl acetate (9 g, 14.674 mmol), (R)-octahydro-2H-pyrido[1,2-a]pyrazine (2.469 g, 17.609 mmol), Cs₂CO₃ (11.9523 g, 36.685 mmol) and BINAP (456.85 mg, 0.734 mmol) in toluene (63 mL) was added Pd(OAc)₂ (329.44 mg, 1.467 mmol) in portions at room temperature under an argon atmosphere. The resulting mixture was stirred for 6 h at 100° C. then the mixture was filtered, the filter cake was washed with EtOAc (100 mL). The filtrate was concentrated under reduced pressure. The residue was purified by prep-TLC (8% MeOH/DCM) to afford the product (6 g, 65% yield) as a solid.

LCMS (ESI) m/z [M+H] calcd C₃₃H₄₅BrN₄O₃: 625.28; found: 627.4

### Intermediate E8. Synthesis of (6³S,4S)-4-amino-1¹-ethyl-2⁵-(fluoromethyl)-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

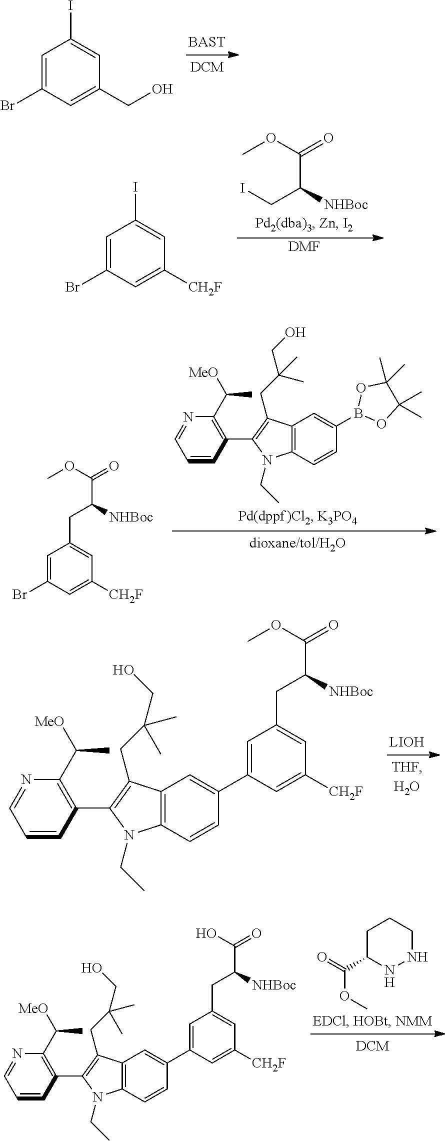

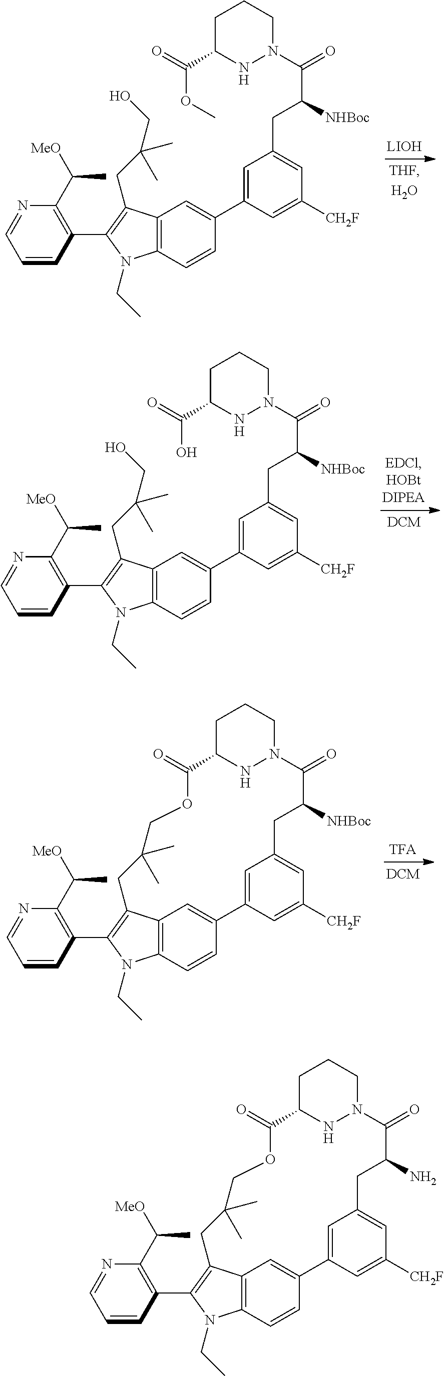

Step 1. To a solution of (3-bromo-5-iodophenyl)methanol (175.0 g, 559.227 mmol) in DCM (2 L) was added BAST (247.45 g, 1118.454 mmol) dropwise at 0° C. The resulting mixture was stirred for 16 h at room temperature. The reaction was quenched with sat. aq. NaHCO₃ at 0° C. The organic layers were washed with H₂O (3×700 mL) and dried over anhydrous Na₂SO₄. After filtration, the filtrate was concentrated under reduced pressure. The residue was purified by silica gel column chromatography (3% EtOAc/pet. ether) to afford the desired product (120 g, 68% yield).

Step 2. Into a 1000 mL 3-necked round-bottom flask was added Zn powder (32.40 g, 495.358 mmol) in DMF (350.0 mL) and 12 (967.12 mg, 3.810 mmol). To the mixture was added a solution of methyl (2R)-2-[(tert-butoxycarbonyl)amino]-3-iodopropanoate (27.0 g, 82.03 mmol) in DMF (10 mL). The mixture was heated to 30° C. for 10 min. To the mixture was then added a solution of methyl (2R)-2-[(tert-butoxycarbonyl)amino]-3-iodopropanoate (54.0 g, 164.07 mmol) in DMF (20 mL). The resulting mixture was stirred for 30 min at room temperature and was filtered. The resulting solution was added to a mixture of 1-bromo-3-(fluoromethyl)-5-iodobenzene (60 g, 190.522 mmol), tris(furan-2-yl)phosphane (2.65 g, 11.431 mmol), and Pd₂(dba)₃ (3.49 g, 3.810 mmol) in DMF (400 mL) at room temperature under argon atmosphere and the reaction mixture was heated to 60° C. for 10 min then removed the oil bath. The resulting mixture was stirred for about 1 h until the temperature cooled down to 50° C. The reaction was quenched with aq. NH₄Cl (3000 mL) and the resulting mixture was extracted with EtOAc (3×1000 mL). The combined organic layers were washed with brine (2×1000 mL) and dried over anhydrous Na₂SO₄. After filtration, the filtrate was concentrated under reduced pressure. The residue was purified by silica gel column chromatography (9% EtOAc/pet. ether) to afford the desired product (45 g, 60% yield).

Step 3. A mixture of methyl (2S)-3-[3-bromo-5-(fluoromethyl)phenyl]-2-[(tert-butoxycarbonyl)amino]propanoate (75.28 g, 192.905 mmol), (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (95 g, 192.905 mmol), Pd(dppf)Cl₂ (14.11 g, 19.291 mmol) and K₂CO₃ (53.32 g, 385.810 mmol) in dioxane (900 mL) and H₂O (180 mL) was stirred for 2 h at 80° C. The resulting mixture was concentrated under reduced pressure and was then diluted with H₂O. The resulting mixture was extracted with EtOAc (3×1200 mL) and the combined organic layers were washed with H₂O (3×500 mL) and dried over anhydrous Na₂SO₄. After filtration, the filtrate was concentrated under reduced pressure. The residue was purified by silica gel column chromatography (50% EtOAc/pet. ether) to afford the desired product (105 g, 80% yield). LCMS (ESI) m/z: [M+H] calcd for C₃₉H₅₀FN₃O₆: 676.38; found 676.1.

Step 4. To a stirred solution of methyl (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoate (108 g, 159.801 mmol) in THE (500 mL) was added a solution of LiOH⋅H₂O (11.48 g, 479.403 mmol) in H₂O (500 mL) at 0° C. The resulting mixture was stirred for 2 h at 0° C. and was then acidified to pH 6 with 1 M HCl (aq.). The mixture was extracted with EtOAc (3×800 mL) and the combined organic layers were washed with brine (2×200 mL) and dried over anhydrous Na₂SO₄. After filtration, the filtrate was concentrated under reduced pressure to afford the desired product (101 g, crude). LCMS (ESI) m/z: [M+H] calcd for C₃₈H₄₈FN₃O₆: 662.36; found 662.1.

Step 5. To a stirred solution of (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoic acid (103 g, 155.633 mmol) and NMM (157.42 g, 1556.330 mmol) in DCM (1200 mL) was added methyl (3S)-1,2-diazinane-3-carboxylate (33.66 g, 233.449 mmol), HOBt (10.51 g, 77.816 mmol) and EDCI (59.67 g, 311.265 mmol) in portions at 0° C. The resulting mixture was stirred a t room temperature for 16 h. The organic layers were then washed with 0.5 M HCl (2×1000 mL) and brine (2×800 mL), dried over anhydrous Na₂SO₄, filtered, and concentrated under reduced pressure. The residue was purified by silica gel column chromatography (50% EtOAc/pet. ether) to afford the desired product (103 g, 83% yield).

LCMS (ESI) m/z: [M+H] calcd for C₄₄H₅₈FN₅O₇: 788.44; found 788.1.

Step 6. To a stirred solution of methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylate (103 g, 130.715 mmol) in THE (700 mL) was added a solution of LiOH⋅H₂O (27.43 g, 653.575 mmol) in H₂O (700 mL) at 0° C. The resulting mixture was stirred for 2 h at 0° C. and was then neutralized to pH 6 with 1 M HCl. The resulting mixture was extracted with EtOAc (3×800 mL) and the combined organic layers were washed with brine (2×600 mL), dried over anhydrous Na₂SO₄, filtered, and concentrated under reduced pressure to afford the desired product (101 g, crude). LCMS (ESI) m/z: [M+H] calcd for C₄₃H₅₆FN₅O₇: 774.43; found 774.1.

Step 7. To a stirred solution of (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylic acid (101 g, 130.50 mmol) in DCM (5500 mL) was added DIPEA (227.31 mL, 1305.0 mmol) and HOBt (88.17 g, 652.499 mmol), and EDCI (375.26 g, 1957.498 mmol) at 0° C. The resulting mixture was stirred at room temperature overnight. The mixture was then washed with 0.5 M HCl (2×2000 mL), brine (2×2000 mL), dried over anhydrous Na₂SO₄, filtered, and concentrated under reduced pressure. The residue was purified by silica gel column chromatography (50% EtOAc/pet. ether) to afford the desired product (68 g, 65% yield). LCMS (ESI) m/z: [M+H] calcd for C₄₃H₅₄FN₅O₆: 756.42; found 756.4.

Step 8. To a stirred solution of tert-butyl ((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)carbamate (350 mg, 0.403 mmol) in DCM (4 mL) was added TFA (1.50 mL) at 0° C. The resulting mixture was stirred at room temperature for 1.5 h and was then concentrated under reduced pressure to afford the desired product (600 mg, crude). LCMS (ESI) m/z: [M+H] calcd for C₃₈H₄₆FN₅O₄: 656.36; found 656.4.

### Intermediate E9. Synthesis of (6³S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione

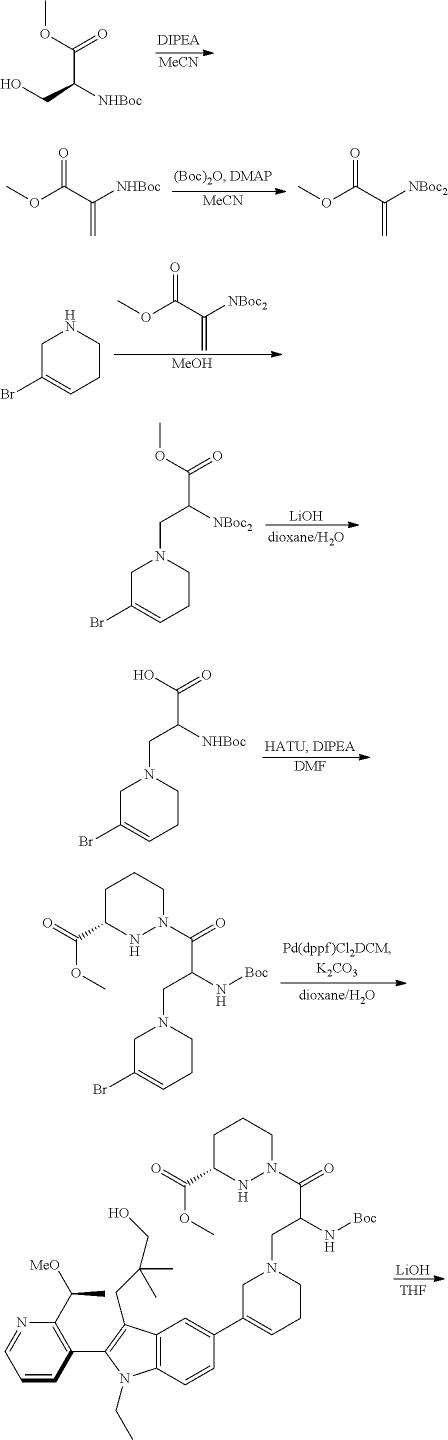

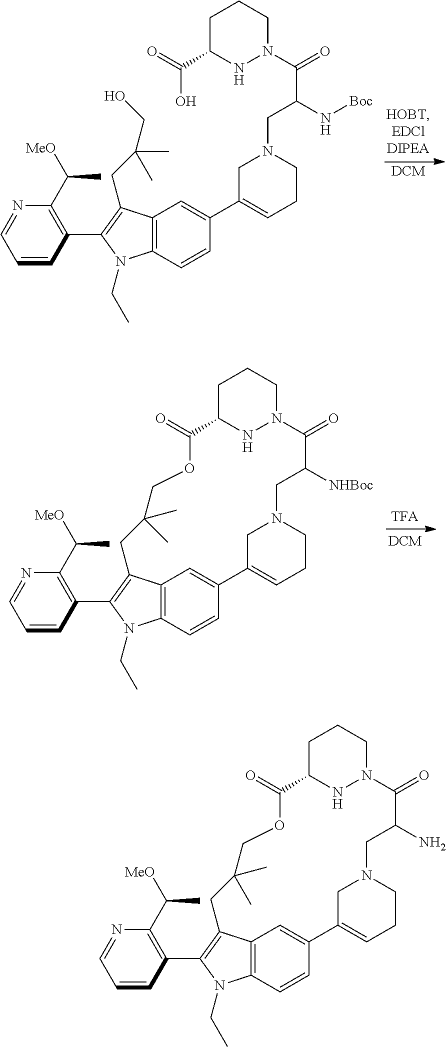

Step 1. To a solution of methyl (tert-butoxycarbonyl)-L-serinate (10 g, 45 mmol) in anhydrous MeCN (150 mL), was added DIPEA (17 g, 137 mmol). The reaction mixture was stirred at 45° C. for 2 h to give the product in solution. LCMS (ESI) m/z [M+Na] calcd for C₉H₁₅NO₄ 201.1; found: 224.1.

Step 2. To a solution of methyl 2-((tert-butoxycarbonyl)amino)acrylate (12 g, 60 mmol) in anhydrous MeCN (150 mL) at 0° C., was added DMAP (13 g, 90 mmol) and (Boc)₂O (26 g, 120 mmol). The reaction was stirred for 6 h, then quenched with H₂O (100 mL) and extracted with DCM (3×200 mL). The combined organic layers were washed with brine (150 mL), dried over anhydrous Na₂SO₄, filtered and concentrated under reduced pressure. The residue was purified by silica gel column chromatography to give the product (12.5 g, 65% yield) as solid. LCMS (ESI) m/z [M+Na] calcd for C₁₄H₂₃NO₆ 301.2; found: 324.1.

Step 3. To a mixture of 5-bromo-1,2,3,6-tetrahydropyridine (8.0 g, 49 mmol) in MeOH (120 mL) under an atmosphere of Ar was added methyl 2-{bis[(tert-butoxy)carbonyl]amino}prop-2-enoate (22 g, 74 mmol). The mixture was stirred for 16 h, then concentrated under reduced pressure and the residue was purified by silica gel column chromatography to give the product (12 g, 47% yield) as an oil. LCMS (ESI) m/z [M+H] calcd for C₁₉H₃₁BrN₂O₆ 462.1; found: 463.1.

Step 4. To a mixture of methyl 2-(bis(tert-butoxycarbonyl)amino)-3-(5-bromo-3,6-dihydropyridin-1(2H)-yl)propanoate (14 g, 30 mmol) in dioxane (30 mL) and H₂O (12 mL) was added LiOH (3.6 g, 151 mmol). The mixture was heated to 35° C. and stirred for 12 h, then 1M HCl was added and the pH adjusted to ˜3-4. The mixture was extracted with DCM (2×300 mL) and the combined organic layers were dried over anhydrous Na₂SO₄ and filtered. The filtrate was concentrated under reduced pressure to give the product (10 g, 85% yield) as a solid. LCMS (ESI) m/z [M+H] calcd for C₁₃H₂₁BrN₂O₄ 348.1; found: 349.0.

Step 5. To a mixture of 3-(5-bromo-3,6-dihydropyridin-1(2H)-yl)-2-((tert-butoxycarbonyl)amino)propanoic acid (10 g, 30 mmol), DIPEA (12 g, 93 mmol) and methyl (3S)-1,2-diazinane-3-carboxylate (5.4 g, 37 mmol) in DMF (100 mL) at 0° C. under an atmosphere of Ar was added HATU (13 g, 34 mmol). The mixture was stirred at 0° C. for 2 h, then H₂O was added and the mixture extracted with EtOAc (2×300 mL). The combined organic layers were dried over anhydrous Na₂SO₄, filtered, the filtrate was concentrated under reduced pressure and the residue was purified by reverse phase chromatography to give the product (9.0 g, 55% yield) as a solid. LCMS (ESI) m/z [M+H] calcd for C₁₉H₃₁BrN₄O₅ 474.1; found: 475.1.

Step 6. A mixture of methyl (3S)-1-(3-(5-bromo-3,6-dihydropyridin-1(2H)-yl)-2-((tert-butoxycarbonyl)amino)propanoyl)hexahydropyridazine-3-carboxylate (9.0 g, 18 mmol), K₂CO₃ (4.5 g, 32 mmol), Pd(dppf)Cl₂. DCM (1.4 g, 2 mmol), 3-(1-ethyl-2-{2-[(1S)-1-methoxyethyl]pyridin-3-yl}-5-(4,4,5,5-tetramethyl-1,3,2-dioxaborolan-2-yl)indol-3-yl)-2,2-dimethylpropan-1-ol (9.8 g, 20 mmol) in dioxane (90 mL) and H₂O (10 mL) under an atmosphere of Ar was heated to 75° C. and stirred for 2 h. H₂O was added and the mixture was extracted with EtOAc (3×200 mL). The combined organic layers were dried over Na₂SO₄, filtered, the filtrate was concentrated under reduced pressure and the residue was purified by silica gel column chromatography to give the product (4.0 g, 25% yield) as a solid. LCMS (ESI) m/z [M+H] calcd for C₄₂H₆₀N₆O₇ 760.5; found: 761.4.

Step 7. To a mixture of methyl (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylate (4.1 g, 5.0 mmol) in THE (35 mL) at 0° C. was added LiOH (0.60 g, 27 mmol). The mixture was stirred at 0° C. for 1.5 h, then 1M HCl added to adjust pH to ˜6-7 and the mixture was extracted with EtOAc (3×200 mL). The combined organic layers were dried over Na₂SO₄, filtered and the filtrate was concentrated under reduced pressure to give the product (3.6 g, 80% yield) as a solid. LCMS (ESI) m/z [M+H] calcd for C₄₁H₅₈N₆O₇ 746.4; found: 747.4.

Step 8. To a mixture of (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylic acid (3.6 g, 5.0 mmol) and DIPEA (24 g, 190 mmol) in DCM (700 mL) under an atmosphere of Ar was added EDCI⋅HCl (28 g, 140 mmol) and HOBt (6.5 g, 50 mmol). The mixture was heated to 30° C. and stirred for 16 h at 30° C., then concentrated under reduced pressure. The residue was diluted with EtOAc (200 mL) and washed with H₂O (2×200 mL), brine (200 mL), dried over Na₂SO₄ and filtered. The filtrate was concentrated under reduced pressure and the residue was purified by silica gel column chromatography to give the product (1.45 g, 40% yield) as a solid. LCMS (ESI) m/z [M+H] calcd for C₄₁H₅NO₆ 728.4; found: 729.4.

Step 9, To a mixture of tert-butyl ((6³S)-1¹-ethyl-1²-(2-((S)-1-methoxyethyl)pyridin-3-yl)-10,10-dimethyl-5,7-dioxo-2¹,2²,2³,2⁶,6¹¹,6²,6³,6⁴,6⁵, 6⁶-decahydro-1¹H-8-oxa-1 (5,3)-indola-6(1,3)-pyridazina-2(5,1)-pyridinacycloundecaphane-4-yl)carbamate (130 mg, 0.20 mmol) in DCM (1.0 mL) at 0° C. was added TFA (0.3 mL). The mixture was warmed to room temperature and stirred for 2 h, then concentrated under reduced pressure to give the product, which was used directly in the next step directly without further purification. LCMS (ESI) m/z [M+H] calcd for C₃₆H₄₈N₆O₄ 628.4; found: 629.4.

### Intermediate E10. Synthesis of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione

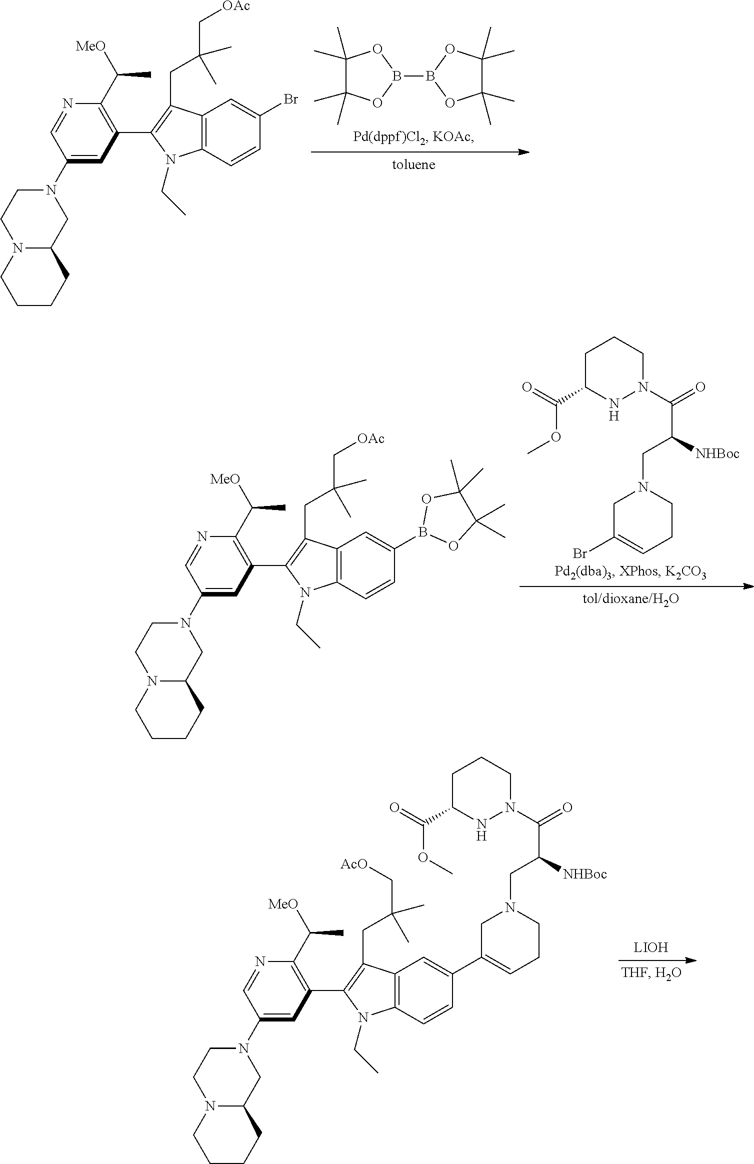

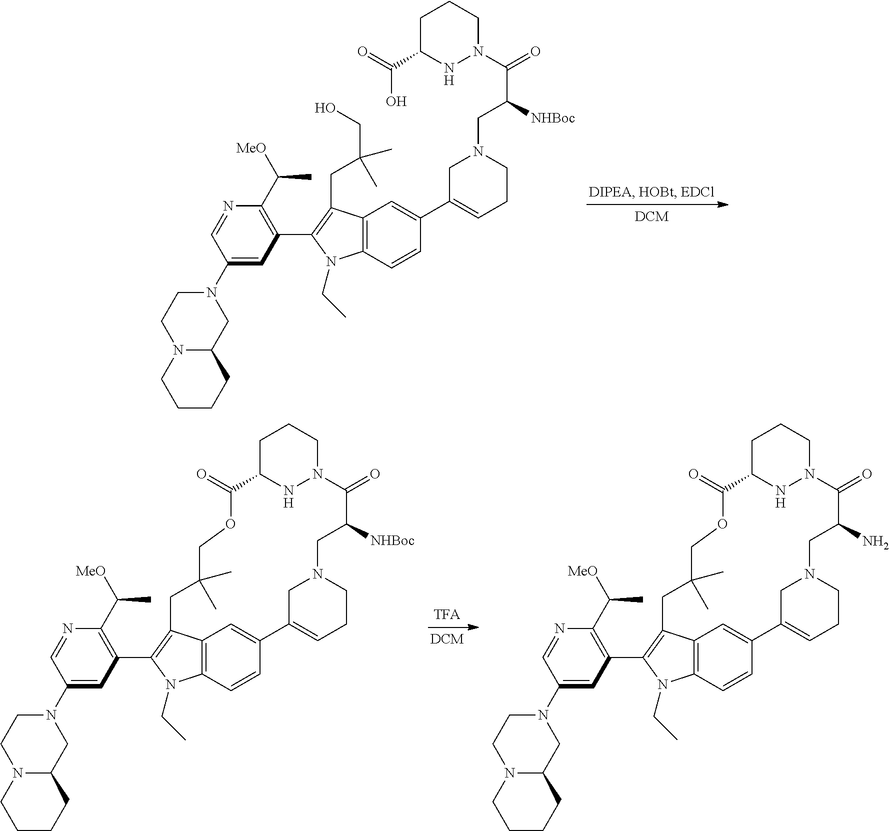

Step 1. To a stirred solution of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (1 g, 1.598 mmol) and B₂Pin₂ (0.81 g, 3.196 mmol) in toluene (20 mL) was added KOAc (0.39 g, 3.995 mmol) and Pd(dppf)Cl₂ (0.12 g, 0.16 mmol). The mixture was stirred for 2 h at 90° C. under a nitrogen atmosphere. The mixture was then basified to pH 8 with sat. aq. NaHCO₃. The resulting mixture was extracted with DCM (3×40 mL) and the combined organic layers were washed with brine (3×40 mL) and dried over anhydrous Na₂SO₄. After filtration, the filtrate was concentrated under reduced pressure. The residue was purified by silica gel column chromatography (2% MeOH/DCM) to afford the product (0.9 g, 83% yield) as a solid.

LCMS (ESI) m/z [M+H] calcd for C₃₉H₅₇BN₄O₅: 673.45; found: 673.6

Step 2. To a stirred solution of 3-(1-ethyl-2-(2-((S)-1-methoxyethyl)-5-((R)-octahydro-2H-pyrido[1,2-a]pyrazin-2-yl)pyridin-3-yl)-5-(4,4,5,5-tetramethyl-1,3,2-dioxaborolan-2-yl)-1H-indol-3-yl)-2,2-dimethylpropyl acetate (0.9 g, 1.338 mmol), methyl (3S)-1-[(2S)-3-(3-bromo-5,6-dihydro-2H-pyridin-1-yl)-2-[(tert-butoxycarbonyl)amino]propanoyl]-1,2-diazinane-3-carboxylate (1.02 g, 2.141 mmol), K₂CO₃ (0.46 g, 3.345 mmol), and X-Phos (0.26 g, 0.535 mmol) in toluene (13.5 mL), dioxane (90 mL), and H₂O (4.5 mL) was added Pd₂(dba)₃ (0.37 g, 0.401 mmol). The mixture was stirred for 2 h at 70° C. under a nitrogen atmosphere. The mixture was then basified to pH 8 with sat. aq. NaHCO₃. The resulting mixture was extracted with DCM (3×100 mL) and the combined organic layers were washed with brine (3×100 mL) and dried over anhydrous Na₂SO₄. After filtration, the filtrate was concentrated under reduced pressure. The residue was purified by silica gel column chromatography (2% MeOH/DCM) to afford the product (1.1 g, 87% yield) as a solid. LCMS (ESI) m/z [M+H] calcd for C₅₂H₇₆N₈O₈: 941.59; found: 941.8 Step 3. To a stirred solution of methyl (S)-1-((S)-3-(5-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)-3,6-dihydropyridin-1(2H)-yl)-2-((tert-butoxycarbonyl)amino)propanoyl)hexahydropyridazine-3-carboxylate (1.1 g, 1.169 mmol) in THE (8 mL) was added a solution of LiOH (0.14 g, 5.845 mmol) in H₂O (8 mL) dropwise at 0° C. under a nitrogen atmosphere. The reaction mixture was stirred for 16 h. The mixture was then acidified to pH 6 with conc. HCl. The resulting mixture was extracted with DCM (3×50 mL) and dried over anhydrous Na₂SO₄. After filtration, the filtrate was concentrated under reduced pressure to afford the product (1.0 g, 96% yield) as a solid, which was used in the next step directly without further purification.

LCMS (ESI) m/z [M+H] calcd for C₄₉H₇₂N₈O₇: 885.56; found: 885.5

Step 4. To a stirred solution of (S)-1-((S)-2-((tert-butoxycarbonyl)amino)-3-(5-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)-3,6-dihydropyridin-1(2H)-yl)propanoyl)hexahydropyridazine-3-carboxylic acid (1.0 g, 1.13 mmol) and HOBt (0.76 g, 5.65 mmol) in DCM (100 mL) was added EDC⋅HCl (6.06 g, 31.64 mmol) and DIPEA (5.11 g, 39.55 mmol) dropwise at 0° C. under a nitrogen atmosphere. The reaction mixture was stirred for 16 h. The mixture was then basified to pH 8 with sat. aq. NaHCO₃. The resulting mixture was extracted with DCM (3×100 mL) and the combined organic layers were washed with brine (3×100 mL) and dried over anhydrous Na₂SO₄. After filtration, the filtrate was concentrated under reduced pressure. The residue was purified by silica gel column chromatography (3% MeOH/DCM) to afford the product (650 mg, 66% yield) as a solid. LCMS (ESI) m/z [M+H] calcd for C₄₉H₇₀N₈O₆: 867.55; found: 867.5

Step 5. To a stirred solution of tert-butyl ((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate (300 mg, 0.346 mmol) in DCM (3 mL) was added TFA (3 mL) dropwise at 0° C. under a nitrogen atmosphere. The resulting mixture was stirred for 1 h at 0° C. The mixture was then basified to pH 8 with sat. aq. NaHCO₃. The resulting mixture was extracted with DCM (3×50 mL) and the combined organic layers were washed with brine (3×50 mL) and dried over anhydrous Na₂SO₄. After filtration, the filtrate was concentrated under reduced pressure to afford the product (260 mg, 98% yield) as a solid, which was used in the next step directly without further purification. LCMS (ESI) m/z [M+H] calcd for C₄₄H₆₂N₈O₄: 767.50; found: 767.2

### Intermediate E11. Synthesis of (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

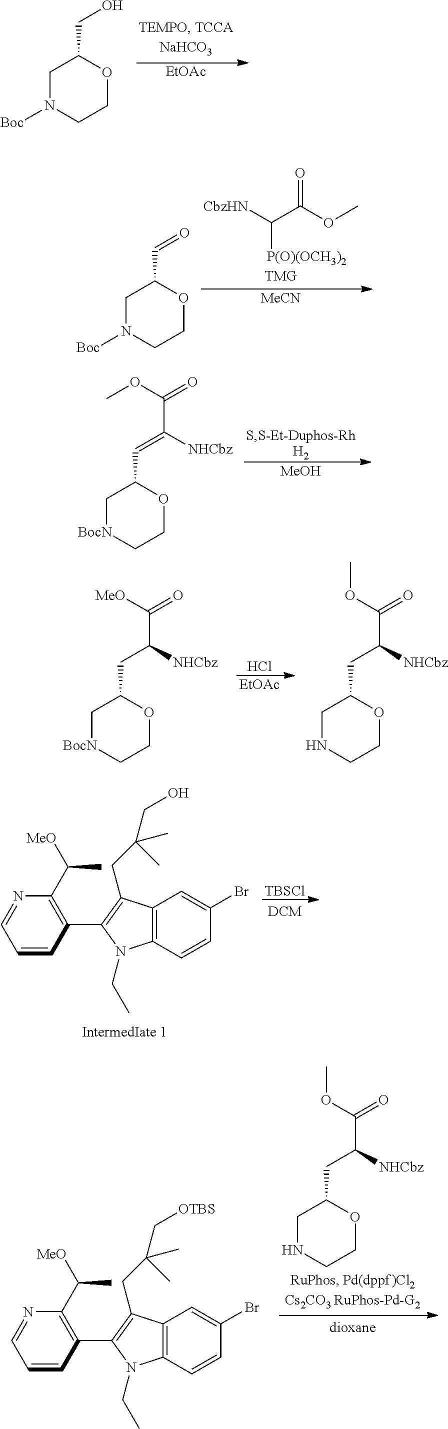

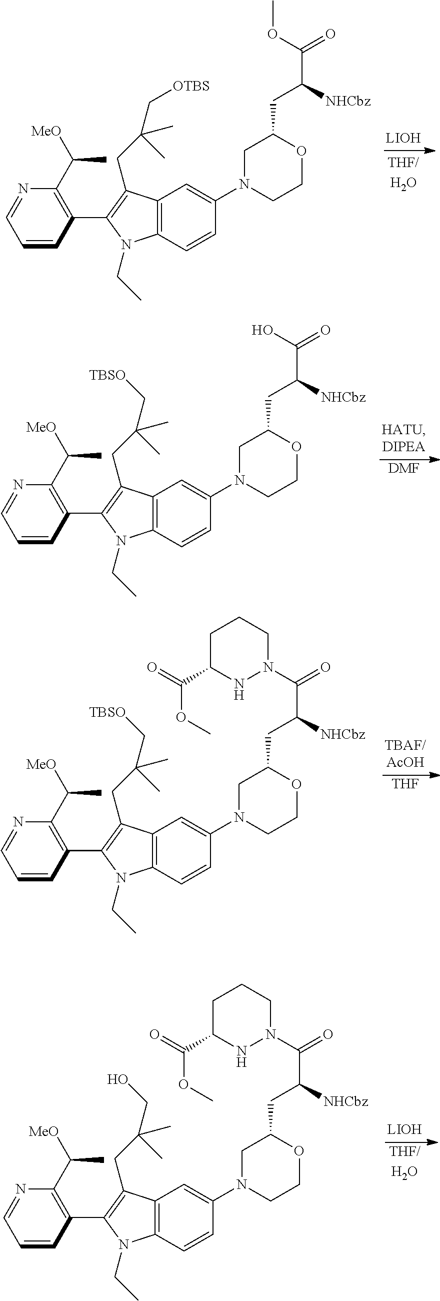

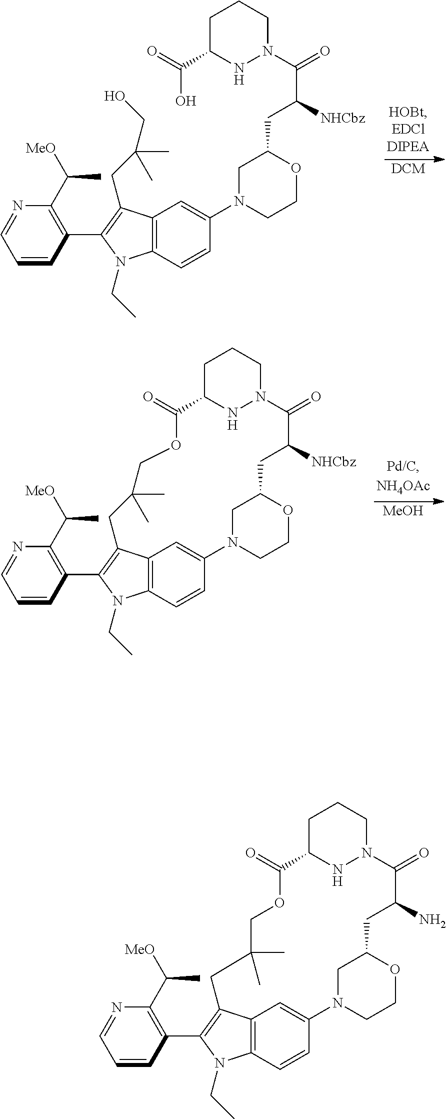

Step 1. To a solution of tert-butyl (2R)-2-(hydroxymethyl)morpholin-4-yl formate (50 g, 230 mmol) in EtOAc (1 L) was added TEMPO (715 mg, 4.6 mmol) and NaHCO₃(58 g, 690 mmol) at room temperature. The mixture was cooled to −50° C., then TCCA (56 g, 241 mmol) in EtOAc (100 mL) was added dropwise over 30 min. The reaction mixture was warmed to 5° C. for 2 h, then quenched with 10% Na₂S₂O₃ (200 mL) and stirred for 20 min. The resulting mixture was filtered and the organic phase was separated. The aqueous phase was extracted with EtOAc (2×100 mL). The combined organic layers were washed with H₂O (100 mL) and brine (100 mL), then dried over anhydrous Na₂SO₄. The organic layer was concentrated under reduced pressure to afford the product (50 g, crude) as an oil.

Step 2. To a solution of tert-butyl (2R)-2-formylmorpholin-4-yl formate (49 g, 153 mmol) and methyl 2-{[(benzyloxy)carbonyl]amino}-2-(dimethoxyphosphoryl)acetate (60 g, 183 mmol) in MeCN (300 mL) was added tetramethylguanidine (35 g, 306 mmol) at 0-10° C. The reaction mixture was stirred at 10° C. for 30 min then warmed to room temperature for 2 h. The reaction mixture was diluted with DCM (200 mL) and washed with 10% citric acid (200 mL) and 10% NaHCO₃ aq. (200 mL). The organic phase was concentrated under reduced pressure, and purified by silica gel column chromatography to afford the product (36 g, 90% yield) as solid. LCMS (ESI) m/z [M+Na] calcd for C₂₁H₂₈N₂O₄ 420.2; found: 443.1 Step 3. To a solution of tert-butyl (S,Z)-2-(2-(((benzyloxy)carbonyl)amino)-3-methoxy-3-oxoprop-1-en-1-yl)morpholine-4-carboxylate (49 g, 0.12 mol) in MeOH (500 mL) was added (S,S)-Et-DUPHOS-Rh (500 mg, 0.7 mmol). The mixture was stirred at room temperature under an H₂ (60 psi) atmosphere for 48 h. The reaction was concentrated and purified by silica gel column chromatography to give the product (44 g, 90% yield) as solid. LCMS (ESI) m/z [M+Na] calcd for C₂₁H₃₀N₂O₇ 422.2; found: 445.2.

Step 4. To a stirred solution of tert-butyl (S)-2-((S)-2-(((benzyloxy)carbonyl)amino)-3-methoxy-3-oxopropyl)morpholine-4-carboxylate (2.2 g, 5.2 mmol) in EtOAc (2 mL) was added HCl/EtOAc (25 mL) at 15° C. The reaction was stirred at 15° C. for 2 h, then concentrated under reduced pressure to afford the product (1.51 g, 90% yield) as an oil. LCMS (ESI) m/z [M+H] calcd for C₁₆H₂₂N₂O₅ 322.1; found: 323.2.

Step 5. To a solution of 3-(5-bromo-1-ethyl-2-{2-[(1 S)-1-methoxyethyl]pyridin-3-yl}indol-3-yl)-2,2-dimethylpropan-1-ol (100 g, 0.22 mol) and imidazole (30.6 g, 0.45 mol) in DCM (800 mL) was added TBSCI (50.7 g, 0.34 mol) in DCM (200 mL) at 0° C. The reaction was stirred at room temperature for 2 h. The resulting solution was washed with H₂O (3×300 mL) and brine (2×200 mL), dried over anhydrous Na₂SO₄, filtered and concentrated under reduced pressure. The residue was purified with silica gel column chromatography to give the product (138 g, 90% yield) as a solid. LCMS (ESI) m/z [M+H] calcd for C₂₉H₄₃BrN₂O₂Si 558.2; found: 559.2.

Step 6. To a stirred solution of (S)-5-bromo-3-(3-((tert-butyldimethylsilyl)oxy)-2,2-dimethylpropyl)-1-ethyl-2-(2-(1-methoxyethyl)pyridin-3-yl)-1H-indole (50 g, 89.3 mmol) in dioxane (500 mL) was added methyl (2S)-2-{[(benzyloxy)carbonyl]amino}-3-[(2S)-morpholin-2-yl]propanoate (31.7 g, 98.2 mmol), RuPhos (16.7 g, 35.7 mmol), di-μ-chlorobis(2-amino-1,1-biphenyl-2-yl-C,N)dipalladium(II) (2.8 g, 4.4 mmol) and cesium carbonate (96 g, 295 mmol) followed by RuPhos-Pd-G2 (3.5 g, 4.4 mmol) at 105° C. under an N₂ atmosphere. The reaction mixture was stirred for 6 h at 105° C. under an N₂ atmosphere. The resulting mixture was filtered, and the filtrate was concentrated under reduced pressure. The residue was purified by prep-TLC chromatography to afford the product (55 g, 73% yield) as a solid. LCMS (ESI) m/z [M+H] calcd for C₄₅H₆₄N₄O₇Si 800.5; found: 801.5.

Step 7. To a solution of methyl (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1 S)-1-methoxyethyl]pyridin-3-yl}indol-5-yl)morpholin-2-yl]propanoate (10 g, 12 mmol) in THE (270 mL) was added LiOH (1.3 g, 31 mmol) in H₂O (45 mL) at room temperature. The reaction was stirred at room temperature for 2 h, then treated with 1N HCl to adjust pH to 4˜5 at 0˜5° C. The resulting mixture was extracted with EtOAc (2×50 mL). The combined organic layers were washed with brine and dried over anhydrous Na₂SO₄. The organic phase was then concentrated under reduced pressure to afford the product (9.5 g, 97% yield) as a solid. LCMS (ESI) m/z [M+H] calcd for C₄₄H₆₂N₄O₇Si 786.4; found: 787.4.

Step 8. To a stirred solution of (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1 S)-1-methoxyethyl]pyridin-3-yl}indol-5-yl)morpholin-2-yl]propanoic acid (10 g, 12.7 mmol) in DMF (150 mL), was added methyl (S)-hexahydropyridazine-3-carboxylate (2 g, 14 mmol), then cooled to 0° C., DIPEA (32.8 g, 254 mmol) was added followed by HATU (9.7 g, 25.4 mmol) at 0-5° C. The reaction mixture was stirred at 0˜5° C. for 1 h. The resulting mixture was diluted with EtOAc (500 mL) and H₂O (200 mL). The organic layer was separated and washed with H₂O (2×100 mL) and brine (100 mL), dried over anhydrous sodium sulfate. The solution was filtered and concentrated under reduced pressure, and the residue was purified by silica gel column chromatography to afford the product. LCMS (ESI) m/z [M+H] calcd for C₅₀H₇₂N₆O₈Si 912.5; found: 913.4.

Step 9. A solution of methyl (S)-1-((S)-2-(((benzyloxy)carbonyl)amino)-3-((S)-4-(3-(3-((tert-butyldimethylsilyl)oxy)-2,2-dimethylpropyl)-1-ethyl-2-(2-((S)-1-methoxyethyl)pyridin-3-yl)-1H-indol-5-yl)morpholin-2-yl)propanoyl)hexahydropyridazine-3-carboxylate (8.5 g, 9 mmol) in THE (8 mL) was added a mixture of tetrabutylammonium fluoride (1M in THF, 180 mL, 180 mmol) and AcOH (11 g, 200 mmol) at room temperature. The reaction mixture was stirred at 75° C. for 3 h. The resulting mixture was diluted with EtOAc (150 mL) and washed with H₂O (6×20 mL). The organic phase was concentrated under reduced pressure to give the product (7.4 g, 100% yield) as solid. LCMS (ESI) m/z [M+H] calcd for C₄₄H₅₈N₆O₈ 799.4; found: 798.4.

Step 10. To a solution of methyl (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl)morpholin-2-yl)propanoyl)hexahydropyridazine-3-carboxylate (8 g, 10 mmol) in THE (200 mL) was added LiOH (600 mg, 25 mmol) in H₂O (30 mL). The reaction mixture was stirred at room temperature for 1 h, then treated with 1 N HCl to adjust pH to 4˜5 at 0˜5° C., and extracted with EtOAc (2×500 mL). The organic phase was washed with brine and concentrated under reduced pressure to afford the product (8 g, crude) as a solid. LCMS (ESI) m/z [M+H] calcd for C₄₃H₅₆N₆O₈ 784.4; found: 785.4.

Step 11. To a stirred solution of (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl)morpholin-2-yl)propanoyl)hexahydropyridazine-3-carboxylic acid (8 g, 10.2 mmol) and DIPEA (59 g, 459 mmol) in DCM (800 mL) was added EDCI (88 g, 458 mmol) and HOBt (27.6 g, 204 mmol) at room temperature under an argon atmosphere. The reaction mixture was stirred at room temperature for 16 h. The resulting mixture was concentrated under reduced pressure, and the residue was purified by silica gel column chromatography to afford the product (5 g, 66% yield) as a solid; LCMS (ESI) m/z [M+H] calcd for C₄₃H₅₄N₆O₇ 766.4; found: 767.4.

Step 12. To a solution of benzyl ((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)- pyridazinacycloundecaphane-4-yl)carbamate (400 mg, 0.5 mmol) in MeOH (20 mL) was added Pd/C (200 mg) and ammonium acetate (834 mg, 16 mmol) at room temperature under an H₂ atmosphere and the mixture was stirred for 2 h. The resulting mixture was filtered and concentrated under reduced pressure. The residue was redissolved in DCM (20 mL) and washed with H₂O (5 mL×2), then concentrated under reduced pressure to afford the product (320 mg, 97% yield) as a solid. LCMS (ESI) m/z [M+H] calcd for C₃₅H₄₈N₆O₅ 632.4; found: 633.3.

### Intermediate E12. Synthesis of (2S)-3-methyl-2-[methyl(4-(prop-2-enoyl)-1-oxa-4,9-diazaspiro[5.5]undecane-9-carbonyl)amino]butanoic acid

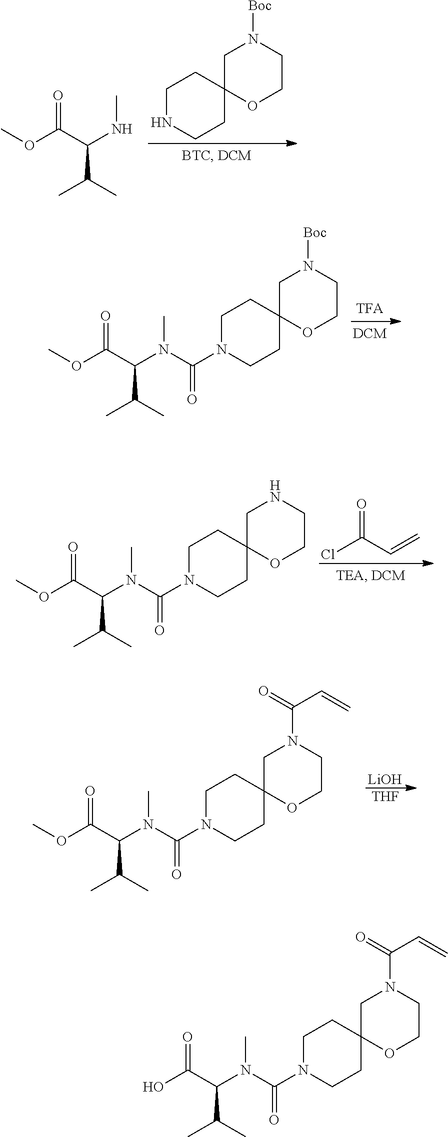

Step 1. To a mixture of ditrichloromethyl carbonate (135 mg, 0.45 mmol) and DCM (1 mL) at 0° C. was added a mixture of methyl (2S)-3-methyl-2-(methylamino)butanoate (200 mg, 1.4 mmol) and pyridine (327 mg, 4.1 mmol) in DCM (1 mL) dropwise. The mixture was stirred at 0° C. for 1 h, then tert-butyl 1-oxa-4,9-diazaspiro[5.5]undecane-4-carboxylate (353 mg, 1.4 mmol), TEA (418 mg, 4.1 mmol) in DCM (2 mL) were added dropwise at 0° C. The mixture was stirred at 0° C. for 1 h, then concentrated under reduced pressure. Brine (20 mL) was added to the residue and the mixture was extracted with DCM (3×20 mL). The combined organic layers were dried over anhydrous Na₂SO₄, filtered, the filtrate was concentrated under reduced pressure and the residue was purified by preparative-HPLC to give tert-butyl 9-{[(2S)-1-methoxy-3-methyl-1-oxobutan-2-yl](methyl)carbamoyl}-1-oxa-4,9-diazaspiro[5.5]undecane-4-carboxylate (335 mg, 57% yield) as a solid. LCMS (ESI): m/z [M+H]⁺ calc'd for C₂₁H₃₇N₃O₆ 427.3; found 428.2.

Step 2. To a mixture of tert-butyl 9-{[(2S)-1-methoxy-3-methyl-1-oxobutan-2-yl](methyl)carbamoyl}-1-oxa-4,9-diazaspiro[5.5]undecane-4-carboxylate (330 mg, 0.77 mmol) in DCM (2.4 mL) at 0° C. was added TFA (0.8 mL). The mixture was stirred at 0° C. for h, then basified to pH ˜7 with saturated NaHCO₃ and the mixture extracted with DCM (3×10 mL). The combined organic layers were washed with brine (3×10 mL), dried over anhydrous Na₂SO₄, filtered and the filtrate was concentrated under reduced pressure to give methyl (2S)-3-methyl-2-[methyl(1-oxa-4,9-diazaspiro[5.5]undecane-9-carbonyl)amino]butanoate (280 mg, crude) as a light yellow solid. LCMS (ESI): m/z [M+H]⁺ calc'd for C₁₆H₂₉N₃O₄ 327.2; found 328.1.

Step 3. To a mixture of methyl (2S)-3-methyl-2-[methyl(1-oxa-4,9-diazaspiro[5.5]undecane-9-carbonyl)amino]butanoate (270 mg, 0.83 mmol) and TEA (1.67 g, 16.5 mmol) in DCM (3 mL) at 0° C. was added acryloyl chloride (75 mg, 0.83 mmol) dropwise. The mixture was stirred at 0° C. for 1 h, then concentrated under reduced pressure and the residue was purified by preparative-HPLC to give methyl (2S)-3-methyl-2-[methyl(4-(prop-2-enoyl)-1-oxa-4,9-diazaspiro[5.5]undecane-9-carbonyl)amino]butanoate (230 mg, 73% yield) as a solid. LCMS (ESI): m/z [M+H]⁺ calc'd for C₁₉H₃₁N₃O₅ 381.2; found 382.2.

Step 4. To a mixture of methyl (2S)-3-methyl-2-[methyl(4-(prop-2-enoyl)-1-oxa-4,9-diazaspiro[5.5]undecane-9-carbonyl)amino]butanoate (220 mg, 0.58 mmol) in THE (1.8 mL) and H₂O (0.6 mL) at 0° C. was added LiOH (21 mg, 0.87 mmol). The mixture was stirred at 0° C. for 1 day, then acidified to pH ˜4 with aqueous HCl and the mixture was extracted with DCM (3×20 mL). The combined organic layers were dried over anhydrous Na₂SO₄, filtered and the filtrate was concentrated under reduced pressure to give (2S)-3-methyl-2-[methyl(4-(prop-2-enoyl)-1-oxa-4,9-diazaspiro[5.5]undecane-9-carbonyl)amino]butanoic acid (137 mg, 65% yield) as a solid. LCMS (ESI): m/z [M+H]⁺ calc'd for C₁₈H₂₉N₃O₅ 367.2; found 368.2.

### Intermediate E13. Synthesis of Methyl N—((S)-3-acryloyl-2-methyl-1-oxa-3,8-diazaspiro[4.5]decane-8-carbonyl)-N-methyl-L-valinate and methyl N—((R)-3-acryloyl-2-methyl-1-oxa-3,8-diazaspiro[4.5]decane-8-carbonyl)-N-methyl-L-valinate

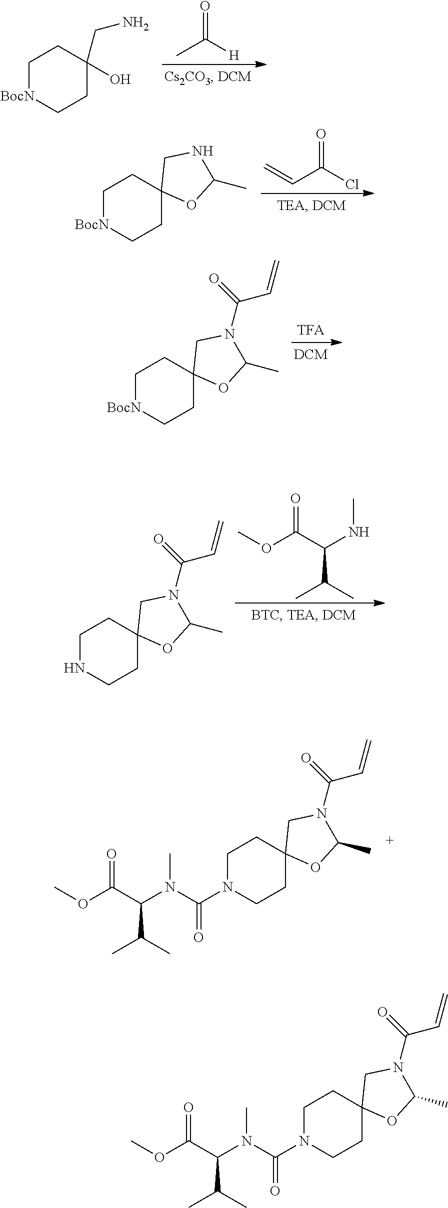

Step 1. To a mixture of tert-butyl 4-(aminomethyl)-4-hydroxypiperidine-1-carboxylate (5.0 g, 21.7 mmol) in DCM (50 mL) was added MgSO₄ (10 g), Cs₂CO₃ (7.07 g, 21.7 mmol) and acetaldehyde (0.96 g, 21.7 mmol). The mixture was stirred at rt for 2 h, then filtered and the filter cake was washed with EtOAc (5×100 mL). The filtrate was concentrated under reduced pressure to give tert-butyl 2-methyl-1-oxa-3,8-diazaspiro[4.5]decane-8-carboxylate (6 g) as an oil, which was used directly in the next step. LCMS (ESI): m/z [M+H]⁺ calc'd for C₁₃H₂₄N₂O₃ 256.2; found 257.4.

Step 2. To a mixture of tert-butyl 2-methyl-1-oxa-3,8-diazaspiro[4.5]decane-8-carboxylate (5.9 g, 23.0 mmol) in DCM (50 mL) at 0° C. was added TEA (6.99 g, 69.1 mmol) and acryloyl chloride (2.08 g, 23.0 mmol). The mixture was stirred at 0° C. for 30 min, then ice/H₂O was added and the mixture extracted with EtOAc (4×30 mL). The combined organic layers were dried over anhydrous Na₂SO₄, filtered, the filtrate was concentrated under reduced pressure and the residue was purified by silica gel column chromatography to give tert-butyl 3-acryloyl-2-methyl-1-oxa-3,8-diazaspiro[4.5]decane-8-carboxylate (2.7 g, 38%) as an oil.

Step 3. To a mixture of tert-butyl 3-acryloyl-2-methyl-1-oxa-3,8-diazaspiro[4.5]decane-8-carboxylate (2.65 g, 8.5 mmol) in DCM (26 mL) at 0° C. was added TFA (13 mL). The mixture was stirred at 0° C. for 1 h, then concentrated under reduced pressure to give 1-(2-methyl-1-oxa-3,8-diazaspiro[4.5]decan-3-yl)prop-2-en-1-one (4.8 g) as an oil. LCMS (ESI): m/z [M+H]⁺ calc'd for C₁₁H₁₈N₂O₂ 210.1; found 211.2.

Step 4. To a mixture of BTC (0.40 g, 1.4 mmol) in DCM (10 mL) at 0° C. was added methyl methyl-L-valinate HCl (0.73 g, 4.1 mmol) and pyridine (1.28 g, 16.2 mmol) in DCM (7 mL). The mixture was stirred at 0° C. for 1 h, then TEA (4.10 g, 40.5 mmol) and 1-(2-methyl-1-oxa-3,8-diazaspiro[4.5]decan-3-yl)prop-2-en-1-one (1.70 g, 8.1 mmol) in DCM were added. The mixture was stirred at 0° C. for 2 h, then ice/H₂O was added and the mixture extracted with EtOAc (3×20 mL). The combined organic layers were dried over anhydrous Na₂SO₄, filtered, and the filtrate was concentrated under reduced pressure. The residue was purified by preparative-TLC and preparative-HPLC to give methyl N—((S)-3-acryloyl-2-methyl-1-oxa-3,8-diazaspiro[4.5]decane-8-carbonyl)-N-methyl-L-valinate (750 mg) and methyl N—((R)-3-acryloyl-2-methyl-1-oxa-3,8-diazaspiro[4.5]decane-8-carbonyl)-N-methyl-L-valinate (730 mg) as an oil. LCMS (ESI): m/z [M+H]⁺ calc'd for C₁₉H₃₁N₃O₅ 381.2; found 382.2.

### Intermediate E14. Synthesis of (2S)-3-methyl-2-{methyl[1-methyl-3-(prop-2-enoyl)-1,3,8-triazaspiro[4.5]decan-8-yl]carbonylamino}butanoic acid, lithium salt

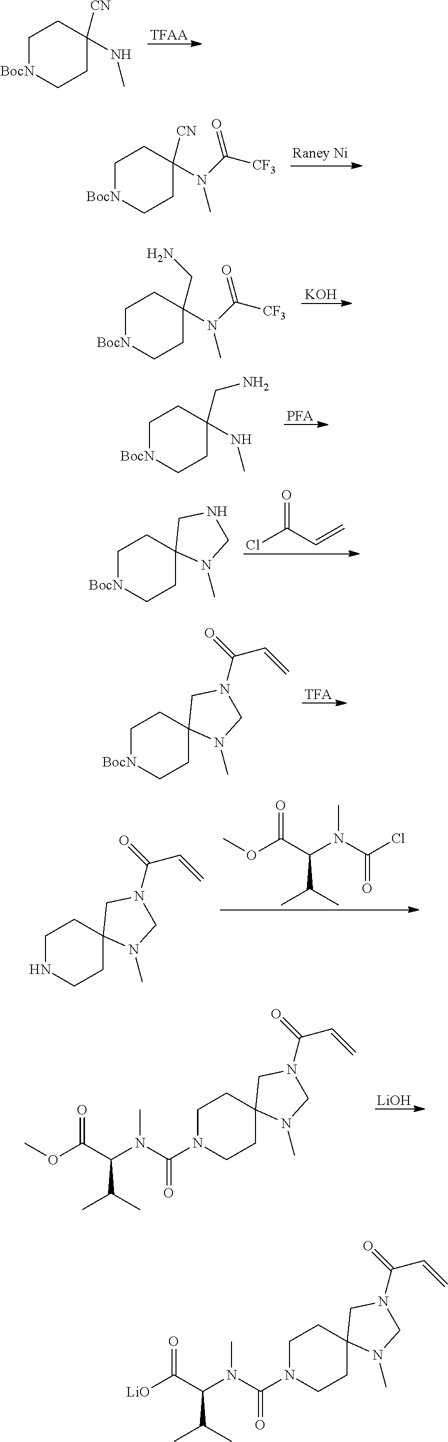

Step 1. To a mixture of tert-butyl [4-cyano-4-(methylamino)piperidin-1-yl] formate (14.4 g, 63 mmol) and pyridine (8 g, 125.6 mmol) in THE (200 mL) at 0° C. was added TFAA (15.8 g, 75.2 mmol). The mixture was warmed to rt and stirred for 1 h, then concentrated under reduced pressure. The residue was dissolved in EtOAc (100 mL), washed with 1 N HCl (100 mL), then dried over Na₂SO₄ and filtered. The filtrate was concentrated under reduced pressure and the crude residue was purified by silica gel column chromatography to give tert-butyl 4-cyano-4-(2,2,2-trifluoro-N-methylacetamido)piperidine-1-carboxylate (15.9 g, 71% yield) as a solid. LCMS (ESI): m/z [M+Na]⁺ calc'd for C₁₄H₂₀F₃N₃NaO₃ 358.1; found 358.2.

Step 2. A mixture of tert-butyl 4-cyano-4-(2,2,2-trifluoro-N-methylacetamido)piperidine-1-carboxylate (9.6 g, 28 mmol) in EtOH (100 mL) and Raney Ni (2 g) was stirred under an atmosphere of H₂ (15 psi) for 16 h. The mixture was filtered, the filtrate was concentrated under reduced pressure and the crude residue was purified by silica gel column chromatography to give tert-butyl 4-(aminomethyl)-4-(2,2,2-trifluoro-methylacetamido)piperidine-1-carboxylate (3.9 g, 40% yield) as a solid. LCMS (ESI): m/z [M+H]⁺ calc'd for C₁₄H₂₄F₃N₃O₃ 339.2; found 340.2.

Step 3. To a mixture of tert-butyl 4-(aminomethyl)-4-(2,2,2-trifluoro-methylacetamido)piperidine-1-carboxylate (3.9 g, 12 mmol) in MeOH (40 mL) and H₂O (8 mL) was added KOH (3.45 g, 60 mmol). The mixture heated to 80° C. and stirred for 1 h, then concentrated under reduced pressure to remove MeOH. The aqueous was extracted with DCM (30 mL×3) and the combined organic layers were dried over Na₂SO₄ and filtered. The filtrate was concentrated under reduced pressure to give tert-butyl 4-(aminomethyl)-4-(methylamino)piperidine-1-carboxylate (2.9 g, 92% yield) as a solid. LCMS (ESI): m/z [M+H]⁺ calc'd for C₁₂H₂₅N₃O₂ 243.2; found 244.2.

Step 4. To a mixture of [4-(aminomethyl)-4-(methylamino)piperidin-1-yl] tert-butyl formate (1.4 g, 5.7 mmol) in Et₂₀ (15 mL) was added formaldehyde (0.77 g, 25.6 mmol). The mixture was stirred at rt for 1 h, then filtered and the filter cake washed with DCM. The filtrate was concentrated under reduced pressure to give tert-butyl {1-methyl-1,3,8-triazaspiro[4.5]decan-8-yl}formate (1.2 g, 77% yield) as an oil.

LCMS (ESI): m/z [M+H]⁺ calc'd for C₁₃H₂₅N₃O₂ 255.2; found 256.3.

Step 5. To a mixture of tert-butyl {1-methyl-1,3,8-triazaspiro[4.5]decan-8-yl}formate (1.4 g, 5.5 mmol), NaHCO₃(1.16 g, 13.7 mmol) in H₂O (15 mL) and DCM (15 mL) at 0° C. was added prop-2-enoyl chloride (0.55 g, 6 mmol). The mixture was stirred at 0° C. for 1H, then H₂O (30 mL) added and the mixture was extracted with DCM (50 mL×3). The obtained organic layers were washed with brine, dried over anhydrous Na₂SO₄ and filtered. The filtrate was concentrated under reduced pressure and the crude residue was purified by silica gel column chromatography to give tert-butyl [1-methyl-3-(prop-2-enoyl)-1,3,8-triazaspiro[4.5]decan-8-yl] formate (0.8 g, 43% yield) as an oil. LCMS (ESI): m/z [M+H]⁺ calc'd for C₁BH₂₇N₃O₃ 309.2; found 310.3.

Step 6. To a mixture of tert-butyl [1-methyl-3-(prop-2-enoyl)-1,3,8-triazaspiro[4.5]decan-8-yl]formate (800 mg, 2.6 mmol) in DCM (6 mL) was added TFA (2 mL). The mixture was stirred at rt for 1 h then concentrated under reduced pressure to give 1-{1-methyl-1,3,8-triazaspiro[4.5]decan-3-yl}prop-2-en-1-one (540 mg), which was used directly in the next step. LCMS (ESI): m/z [M+H]⁺ calc'd for C₁₁H₁₉N₃O 209.2; found 210.3.

Step 7. To a mixture of 1-{1-methyl-1,3,8-triazaspiro[4.5]decan-3-yl}prop-2-en-1-one (540 mg, 2.6 mmol) and methyl (2S)-2-[(chlorocarbonyl)(methyl)amino]-3-methylbutanoate (589 mg, 2.83 mmol) in DCM (10 mL) at 0° C. was added TEA (781 mg, 7.74 mmol). The mixture was stirred at 0° C. for 1 h, then H₂O (30 mL) added and the mixture was extracted with DCM (50 mL×3). The obtained organic layers were washed with brine, dried over anhydrous Na₂SO₄ and filtered. The filtrate was concentrated under reduced pressure and the crude residue was purified by silica gel column chromatography to give methyl (2S)-3-methyl-2-{methyl[1-an oil. LCMS (ESI): m/z [M+H]⁺ calc'd for C₁₉H₃₂N₄O₄ 380.2; found 381.3.

Step 8. To a mixture of methyl (2S)-3-methyl-2-{methyl[1-methyl-3-(prop-2-enoyl)-1,3,8-triazaspiro[4.5]decan-8-yl]carbonylamino}butanoate (600 mg, 1.6 mmol) in THE (3 mL) was added LiOH (75.5 mg, 3.15 mmol) in H₂O (2 mL). The mixture was stirred at rt for 1 h, then lyophilized to afford (2S)-3-methyl-2-{methyl[1-methyl-3-(prop-2-enoyl)-1,3,8-triazaspiro[4.5]decan-8-yl]carbonylamino}butanoic acid, lithium salt (500 mg, 78% yield) as a solid. LCMS (ESI): m/z [M+H]⁺ calc'd for C₁₈H₃₀N₄O₄ 366.2; found 367.2.

### Intermediate E15. Synthesis of (2S)-3-methyl-2-{methyl[3-(prop-2-enoyl)-1-oxa-3,8-diazaspiro[4.5]decan-8-yl]carbonylamino}butanoic acid

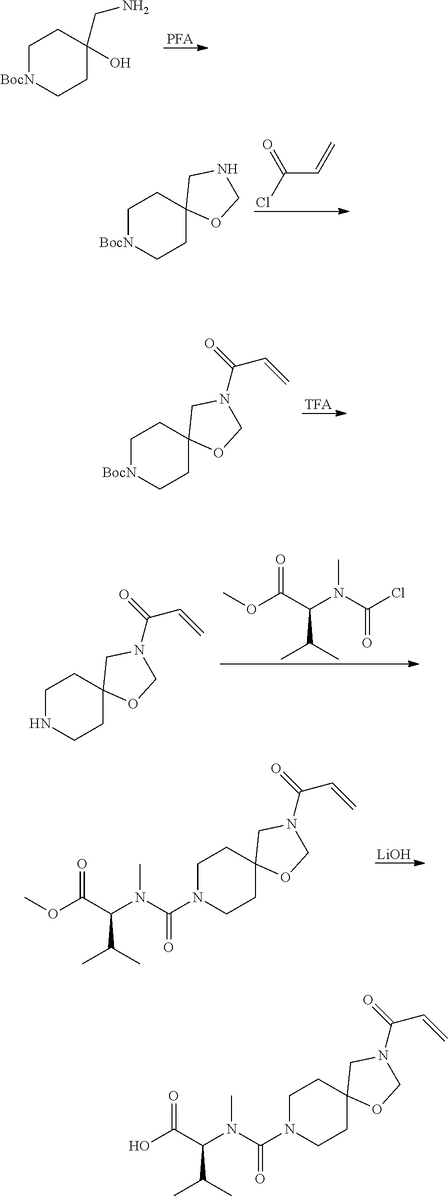

Step 1. To a mixture of tert-butyl 4-(aminomethyl)-4-hydroxypiperidine-1-carboxylate (26 g, 112.9 mmol) in MeOH (52 mL) and 3M NaOH (260 mL) was added HCHO (37 wt. % in H₂O; 52 mL). The mixture was stirred for stirred at rt for 16 h, then extracted with DCM (100 mL×3). The combined organic layers were dried over Na₂SO₄, filtered and concentrated under reduced pressure to give tert-butyl 1-oxa-3,8-diazaspiro[4.5]decane-8-carboxylate (28.8 g) as an oil. The crude product was used directly in the next step. LCMS (ESI): m/z [M+H]⁺ calc'd for C₁₂H₂₂N₂O₃ 242.2; found 243.2.

Step 2. To a mixture of tert-butyl 1-oxa-3,8-diazaspiro[4.5]decane-8-carboxylate (14.4 g, 59.4 mmol) and NaHCO₃(14.97 g, 178.2 mmol) in DCM (75 mL) and H₂O (75 mL) at 0° C. was added prop-2-enoyl chloride (8.06 g, 89.1 mmol). The mixture was stirred at 0° C. for 1 h, then extracted with DCM (50 mL×3). The combined organic layers were concentrated under reduced pressure and the crude residue was purified by silica gel column chromatography to give tert-butyl 3-(prop-2-enoyl)-1-oxa-3,8-diazaspiro[4.5]decane-8-carboxylate (10 g, 54% yield) as an oil. LCMS (ESI): m/z [M+H]⁺ calc'd for C₁₅H₂₄N₂O₄ 296.2; found 297.2.

Step 3. To a mixture of tert-butyl 3-(prop-2-enoyl)-1-oxa-3,8-diazaspiro[4.5]decane-8-carboxylate (1.0 g, 3.4 mmol) in DCM (6 mL) was added TFA (2 mL). The mixture was stirred at rt for 1 h, then concentrated under reduced pressure to give 1-{1-oxa-3,8-diazaspiro[4.5]decan-3-yl}prop-2-en-1-one (0.67 g) as an oil. The product was used to next step directly. LCMS (ESI): m/z [M+H]⁺ calc'd for C₁₀H₁₆N₂O₂ 196.1; found 197.1.

Step 4. To a mixture of methyl (2S)-2-[(chlorocarbonyl)amino]-3-methylbutanoate (0.66 g, 3.4 mmol) and TEA (1.72 g, 17 mmol) in DCM (10 mL) at 0° C. was added 1-{1-oxa-3,8-diazaspiro[4.5]decan-3-yl}prop-2-en-1-one (0.67 g, 3.4 mmol). The mixture was stirred at 0° C. for 1 h, then H₂O (30 mL) added and the mixture was extracted with DCM (30 mL). The combined organic layers were concentrated under reduced pressure and the crude residue was purified by silica gel column chromatography to give methyl (2S)-3-methyl-2-{methyl[3-(prop-2-enoyl)-1-oxa-3,8-diazaspiro[4.5]decan-8-yl]carbonylamino}butanoate (600 mg, 47% yield) as an oil. LCMS (ESI): m/z [M+H]⁺ calc'd for C₁₈H₂₉N₃O₅ 367.2; found 368.3.

Step 5. To a mixture of methyl (2S)-3-methyl-2-{methyl[3-(prop-2-enoyl)-1-oxa-3,8-diazaspiro[4.5]decan-8-yl]carbonylamino}butanoate (600 mg, 1.63 mmol) in THE (5 mL) was added a solution of lithium hydroxide (78 mg, 3.3 mmol) in H₂O (5 mL). The mixture was stirred at rt for 4 h, then adjusted to pH ˜4 with 1 N HCl, and extracted with DCM (20 mL×3). The combined organic layers were concentrated under reduced pressure to give (2S)-3-methyl-2-{methyl[3-(prop-2-enoyl)-1-oxa-3,8-diazaspiro[4.5]decan-8-yl]carbonylamino}butanoic acid (500 mg) as an oil. LCMS (ESI): m/z [M+H]⁺ calc'd for C₁₇H₂₇N₃O₅ 353.2; found 354.2.

### Intermediate E16. Synthesis of (2S)-3-methyl-2-{methyl[4-(prop-2-enoyl)-1-propyl-1,4,9-triazaspiro[5.5]undecan-9-yl]carbonylamino}butanoic acid, lithium salt

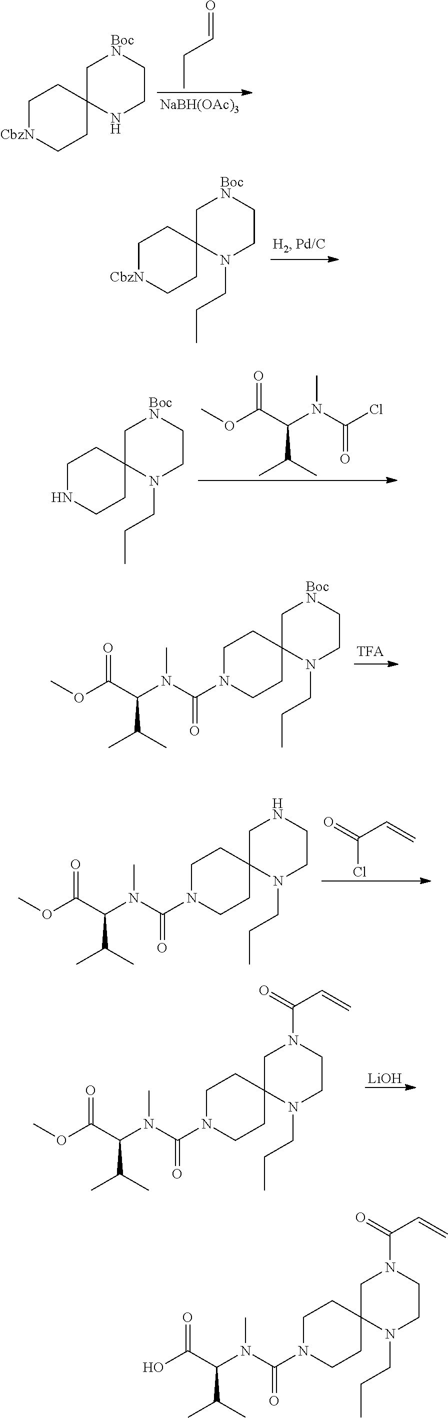

Step 1. To a mixture of tert-butyl 9-{3-[(formyloxy)methyl]phenyl}-1,4,9-triazaspiro[5.5]undecane-4-carboxylate (1.0 g, 2.6 mmol) and propanal (0.3 g, 5.2 mmol) in DCM (10 mL) was stirred at rt for 20 min. NaBH(OAc)₃ (1.1 g, 5.2 mmol) was added and the mixture was stirred at rt for 1 h, then H₂O (20 mL) added and the mixture was extracted with DCM (20 mL×3). The combined organic layers were washed with brine (20 mL), dried over anhydrous Na₂SO₄ and filtered. The filtrate was concentrated under reduced pressure and the crude residue was purified by silica gel column chromatography to give tert-butyl 9-{3-[(formyloxy)methyl]phenyl}-1-propyl-1,4,9-triazaspiro[5.5]undecane-4-carboxylate (0.7 g, 62% yield) as an oil. LCMS (ESI): m/z [M+H]⁺ calc'd for C₂₄H₃₇N₃O₄ 431.3; found 432.3.

Step 2. A mixture of tert-butyl 9-{3-[(formyloxy)methyl]phenyl}-1-propyl-1,4,9-triazaspiro[5.5]undecane-4-carboxylate (600 mg, 1.39 mmol) and 10% Pd/C (148 mg, 1.39 mmol) in THE (10 mL) was stirred under an atmosphere of H2 (15 psi) at rt for 1 h. The mixture was filtered and the filtrate was concentrated under reduced pressure to give tert-butyl 1-propyl-1,4,9-triazaspiro[5.5]undecane-4-carboxylate (500 mg) as an oil. LCMS (ESI): m/z [M+H]⁺ calc'd for C₁₆H₃₁N₃O₂ 297.2; found 298.2.

Step 3. To a mixture of methyl (2S)-2-[(chlorocarbonyl)(methyl)amino]-3-methylbutanoate (314 mg, 1.5 mmol) in DCM (5 mL) at 0° C. was added TEA (458 mg, 4.5 mmol) and tert-butyl 1-propyl-1,4,9-triazaspiro[5.5]undecane-4-carboxylate (450 mg, 1.5 mmol). The mixture was stirred at 0° C. for 1 h, then H₂O (20 mL) added and the mixture was extracted with DCM (20 mL×3). The combined organic layers were washed with brine (20 mL), dried over anhydrous Na₂SO₄ and filtered. The filtrate was concentrated under reduced pressure and the crude residue was purified by silica gel column chromatography to give tert-butyl 9-{[(2S)-1-methoxy-3-methyl-1-oxobutan-2-yl](methyl)carbamoyl}-1-propyl-1,4,9-triazaspiro[5.5]undecane-4-carboxylate (650 mg, 83% yield) as an oil. LCMS (ESI): m/z [M+H]⁺ calc'd for C₂₄H₄₄N₄O₅ 468.3; found 469.3.

Step 4. To a mixture of tert-butyl 9-{[(2S)-1-methoxy-3-methyl-1-oxobutan-2-yl](methyl)carbamoyl}-1-propyl-1,4,9-triazaspiro[5.5]undecane-4-carboxylate (550 mg, 1.17 mmol) in DCM (6 mL) at 0° C. was added TFA (2 mL). The mixture was stirred at 0° C. for 15 min, then concentrated under reduced pressure to give methyl (2S)-3-methyl-2-[methyl({1-propyl-1,4,9-triazaspiro[5.5]undecan-9-yl}carbonyl)amino]butanoate (435 mg), that was used directly in the next step. LCMS (ESI): m/z [M+H]⁺ calc'd for C₁₉H₃₆N₄O₃ 368.3; found 369.3.

Step 5. To a mixture of methyl (2S)-3-methyl-2-[methyl({1-propyl-1,4,9-triazaspiro[5.5]undecan-9-yl}carbonyl)amino]butanoate (435 mg, 1.18 mmol) in DCM (5 mL) and H₂O (5 mL) at 0° C. was added NaHCO₃ (991 mg, 11.8 mmol) and prop-2-enoyl chloride (214 mg, 2.36 mmol). The mixture was stirred at 0° C. for 1 h, then H₂O (20 mL) added and the mixture was extracted with DCM (20 mL×3). The combined organic layers were washed with brine (20 mL), dried over anhydrous Na₂SO₄ and filtered. The filtrate was concentrated under reduced pressure and the crude residue was purified by silica gel column chromatography to give methyl (2S)-3-methyl-2-{methyl[4-(prop-2-enoyl)-1-propyl-1,4,9-triazaspiro[5.5]undecan-9-yl]carbonylamino}butanoate (460 mg, 83% yield) as an oil. LCMS (ESI): m/z [M+H]⁺ calc'd for C₂₂H₃₈N₄O₄ 422.3; found 423.3.

Step 6. To a mixture of methyl (2S)-3-methyl-2-{methyl[4-(prop-2-enoyl)-1-propyl-1,4,9-triazaspiro[5.5]undecan-9-yl]carbonylamino}butanoate (100 mg, 0.24 mmol) in THE (1 mL) was added a mixture of LiOH (11.3 mg, 0.47 mmol) in H₂O (1.5 mL). The mixture was stirred at rt for 4 h, then lyophilized to afford (2S)-3-methyl-2-{methyl[4-(prop-2-enoyl)-1-propyl-1,4,9-triazaspiro[5.5]undecan-9-yl]carbonylamino}butanoic acid, lithium salt (96 mg) as a solid, that was used directly in the next step.

LCMS (ESI): m/z [M+H]⁺ calc'd for C₂₁H₃₆N₄O₄ 408.3; found 409.3.

### Intermediate E17. Synthesis of N—((S)-3-acryloyl-4-methyl-1-oxa-3,8-diazaspiro[4.5]decane-8-carbonyl)-N-methyl-L-valine methyl ester

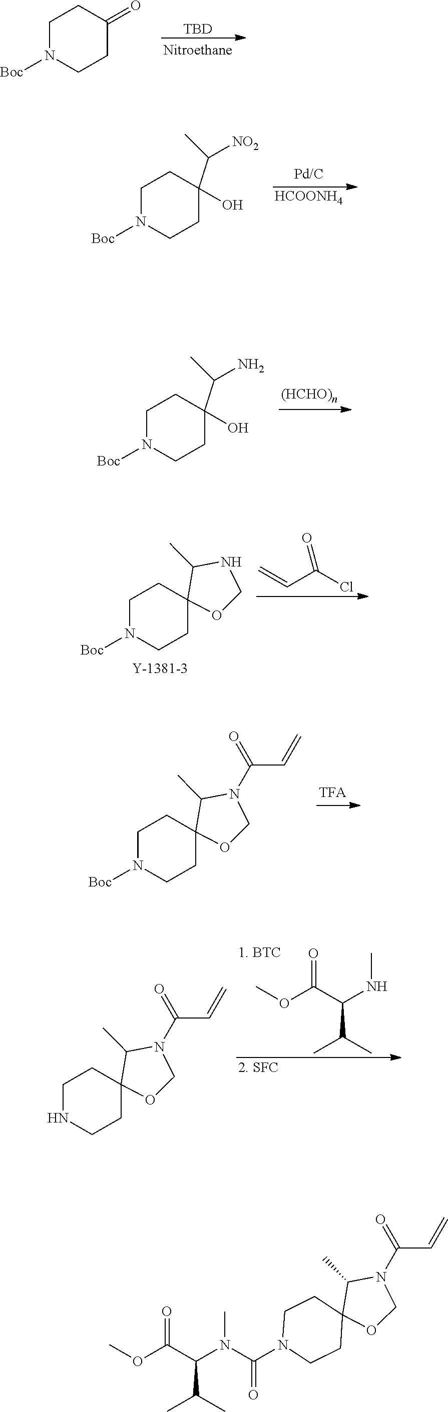

Step 1. To a solution of nitroethane (1 L) was added tert-butyl (4-oxopiperidin-1-yl) formate (200 g, 1 mol, 1 eq) and TBD (13.9 g, 0.1 mol, 0.1 eq) at 0° C. The reaction mixture was stirred at 20° C. for 16 h. The resulting mixture was concentrated under reduced pressure and the remaining residue was purified by silica gel column chromatography to afford tert-butyl 4-hydroxy-4-(1-nitroethyl)piperidine-1-carboxylate (135 g, yield 49%) as a white solid. ESI-MS m/z=299.2 [M+H]⁺, Calculated MW: 274.15.

Step 2. To a solution of tert-butyl 4-hydroxy-4-(1-nitroethyl)piperidine-1-carboxylate (135 g, 0.49 mol, 1 equiv) and HCOONH₄ (269 g, 4.3 mol, 8.7 equiv) in MeOH (1350 mL) was added Pd/C (13.6 g, 0.13 mol, 0.26 equiv) and AcOH (0.29 g, 4.9 mmol, 0.01 equiv) at room temperature. The reaction mixture was stirred for 16 h after which the mixture was adjusted to pH value of 8 with TEA (4.96 g, 0.1 equiv) and filtered. The filter cake was washed with DCM/MeOH (200 mL, 5/1). The filtrate was concentrated under reduced pressure and purified by alkaline silica gel column chromatography to afford tert-butyl 4-(1-aminoethyl)-4-hydroxypiperidine-1-carboxylate (135 g, yield 89%) as a white solid. ESI-MS m/z=189.3 [M+H−tBu]⁺, Calculated MW: 244.34.

Step 3. To a solution of [4-(1-aminoethyl)-4-hydroxypiperidin-1-yl] tert-butyl formate (40 g, 0.16 mol, 1 eq) in ACN (800 mL) was added MgSO₄ (39.1 g, 0.33 mol, 2 eq), Cs₂CO₃ (79.7 g, 0.25 mol, 1.5 eq) and (HCHO)n (19.6 g, 0.65 mol, 4 eq). The mixture was stirred at 50° C. for 2 h under N₂. The reaction mixture was filtered and the filtrate was concentrated in vacuo to afford tert-butyl {4-methyl-1-oxa-3,8-diazaspiro[4.5]decan-8-yl}formate (40 g, yield 97%) as a colorless oil. ESI-MS m/z=257.3 [M+H]⁺, Calculated MW: 256.35.

Step 4. To a mixture of tert-butyl {4-methyl-1-oxa-3,8-diazaspiro[4.5]decan-8-yl}formate (40 g, 155.4 mmol, 1 eq) and NaHCO₃(52.2 g, 621.6 mmol, 3 eq) in DCM (500 mL) and H₂O (500 mL) was added prop-2-enoyl chloride (15.5 g, 170.9 mmol, 1 eq) dropwise at 0° C. and stirred at 0° C. for 1 h. The resulting was filtered, and the filtrate was extracted with DCM (200 mL×2). The organic phase was washed with brine (100 mL) and concentrated under reduced pressure. The resulting residue was purified by column chromatography to afford give tert-butyl [4-methyl-3-(prop-2-enoyl)-1-oxa-3,8-diazaspiro[4.5]decan-8-yl] formate (33 g, yield 68%) as a colorless oil. ESI-MS m/z=311.1 [M+H]⁺, Calculated MW: 310.39.

Step 5. A mixture of tert-butyl [4-methyl-3-(prop-2-enoyl)-1-oxa-3,8-diazaspiro[4.5]decan-8-yl]formate (200 g, 0.64 mol, 1 equiv) in TFA/DCM (700 ml, ⅓, 2 L) was stirred for 1 h at 0° C. The mixture was concentrated under reduced pressure at 0˜10° C. to afford crude 1-(4-methyl-1-oxa-3,8-diazaspiro[4.5]decan-3-yl)prop-2-en-1-one (350 g TFA salt, purity 36%). ESI-MS m/z=211.2 [M+H]⁺, Calculated MW: 210.28.

Step 6. To a solution of methyl (2S)-3-methyl-2-(methylamino)butanoate (63 g, 0.345 mol, 1 eq) and DIEA (360 g, 2.8 mol, 8 eq) in DCM (600 mL) was added BTC (36.5 g, 0.14 mol, 0.4 eq) in portions at 0° C., and the mixture was stirred at 0° C. for 1 h. The reaction mixture was then cooled to −40° C. and a solution of 1-{4-methyl-1-oxa-3,8-diazaspiro[4.5]decan-3-yl}prop-2-en-1-one (TFA salt, 36%, 175 g, 0.32 mol, 0.92 eq) in 300 ml DCM was added dropwise. The reaction mixture was then allowed to warm to rt and stirred for 12 h at rt. The reaction mixture was then concentrated under reduced pressure and the remaining residue was diluted with EA (0.5 L). The mixture was washed with brine (200 ml×2), dried over Na₂SO₄, and concentrated under reduced pressure to afford crude residue. The residue was purified by chromatography to afford methyl N-(3-acryloyl-4-methyl-1-oxa-3,8-diazaspiro[4.5]decane-8-carbonyl)-N-methyl-L-valinate as a racemic mixture (168 g, 64% yield). A portion of the racemic product (85 g) was separated using chiral SFC to afford N—((S)-3-acryloyl-4-methyl-1-oxa-3,8-diazaspiro[4.5]decane-8-carbonyl)-N-methyl-L-valine methyl ester ESI-MS m/z=382.2 [M+H]⁺, Calculated MW: 381.2. ¹H NMR (400 MHz, CD₃OD) δ 6.72-6.24 (m, 2H), 5.85-5.70 (m, 1H), 5.22-4.99 (m, 2H), 4.01 (d, J=6.5 Hz, 1H), 3.88 (d, J=10.4 Hz, 1H), 3.69 (s, 3H), 3.51-3.40 (m, 2H), 3.25-3.06 (m, 2H), 2.96 (s, 3H), 2.26-2.15 (m, 1H), 1.82-1.63 (m, 4H), 1.19 (dd, J=6.5, 2.3 Hz, 3H), 0.95 (dd, J=12.3, 6.6 Hz, 6H).

### Intermediate E18. Synthesis of Methyl (2S)-2-[(3-{3-[(formyloxy)methyl]phenyl}-1-oxa-3,8-diazaspiro[4.5]decan-8-yl)carbonyl(methyl)amino]-3-methylbutanoate

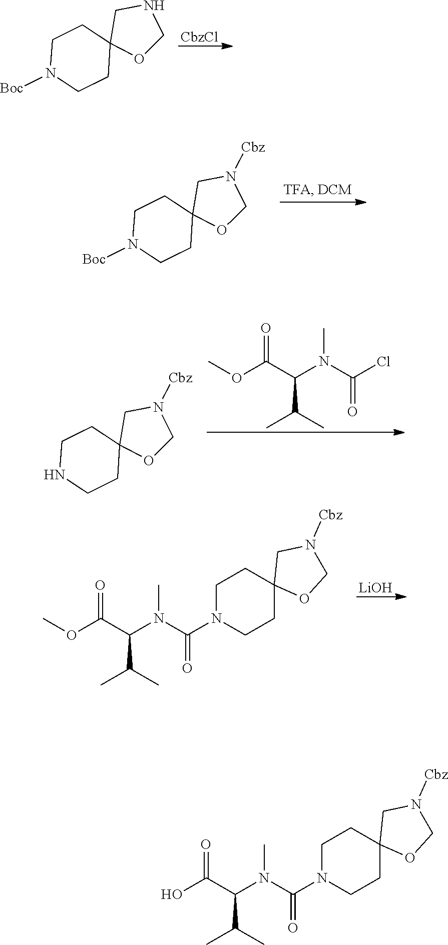

Step 1. To a solution of tert-butyl {1-oxa-3,8-diazaspiro[4.5]decan-8-yl}formate (2 g, 8.2 mmol, 1 eq) and NaHCO₃(2.1 g, 25 mmol, 3 eq) in DCM/H₂O=1/1 (20 mL) was added CbzCl (1.7 g, 9.8 mmol, 1.2 eq). The mixture was stirred at 0° C. for 20 min. The reaction mixture was treated with H₂O (20 mL), extracted with DCM (30 mL×3). The combined organic layers were washed with water (20 mL) and brine (20 mL) and then dried over Na₂SO₄, filtered, and concentrated under reduced pressure to afford the crude product. The crude product was purified by chromatography to afford tert-butyl (3-{3-[(formyloxy)methyl]phenyl}-1-oxa-3,8-diazaspiro[4.5]decan-8-yl) formate (2 g, 61% yield) as a colorless oil. ESI-MS m/z: 399.3 [M+Na]⁺; Calculated MW: 376.2

Step 2. To a solution of tert-butyl (3-{3-[(formyloxy)methyl]phenyl}-1-oxa-3,8-diazaspiro[4.5]decan-8-yl) formate (2 g, 5.3 mmol, 1 eq) in DCM (12 mL) was added TFA (6 g, 53 mmol, 10 eq) at 20° C. The reaction mixture was stirred at 20° C. for 40 min. The reaction mixture was then concentrated to afford a yellow oil. The yellow oil was dissolved in DCM (30 ml) and adjusted with saturated NaHCO₃ aqueous to pH=8˜9. The resulting mixture was extracted with DCM (30 mL×3) and the combined organic layers were washed with water (20 mL), and brine (20 mL). The organic layer was dried over Na₂SO₄, filtered and concentrated under reduced pressure to afford (3-{1-oxa-3,8-diazaspiro[4.5]decan-3-yl}phenyl)methyl formate (1.5 g, 98% yield) as a white solid. ESI-MS m/z: 277.3 [M+H]⁺; Calculated MW: 276.2

Step 3. To a solution of methyl (2S)-2-[(chlorocarbonyl)(methyl)amino]-3-methylbutanoate (1.1 g, 5.1 mmol, 1 eq) and TEA (1.6 g, 15 mmol, 3 eq) in DCM (20 mL) was added (3-{1-oxa-3,8-diazaspiro[4.5]decan-3-yl}phenyl)methyl formate (1.4 g, 5.1 mmol, 1 eq). The mixture was stirred at 0° C. for 0.5 h. The mixture was treated with H₂O (20 mL), extracted with DCM (30 mL×3) and the combined organic layers were washed with water (20 mL), and brine (20 mL). The organic layer was dried over Na₂SO₄, filtered and concentrated under reduced pressure to afford crude product. The crude product was purified by chromatography to afford methyl (2S)-2-[(3-{3-[(formyloxy)methyl]phenyl}-1-oxa-3,8-diazaspiro[4.5]decan-8-yl)carbonyl(methyl)amino]-3-methylbutanoate (1.7 g, 70% yield) as yellow oil.

ESI-MS m/z: 448.3 [M+H]⁺, Calculated MW: 447.2

Step 4. To a solution of methyl (2S)-2-[(3-{3-[(formyloxy)methyl]phenyl}-1-oxa-3,8-diazaspiro[4.5]decan-8-yl)carbonyl(methyl)amino]-3-methylbutanoate (400 mg, 0.89 mmol, 1 eq) in THF(1 mL) was added a solution of LiOH (64 mg, 2.7 mmol, 3 eq) in H₂O (1.5 mL). The mixture was stirred at 20° C. for 12 h. The resulting solution was adjusted pH=6 with 1N HCl and extracted with DCM (30 mL×3). The combined organic layers were washed with water (20 mL), and brine (20 mL) and dried over Na₂SO₄, filtered and concentrated under reduced pressure to give (2S)-2-[(3-{3-[(formyloxy)methyl]phenyl}-1-oxa-3,8-diazaspiro[4.5]decan-8-yl)carbonyl(methyl)amino]-3-methylbutanoic acid (380 mg, 88% yield) as yellow oil. ESI-MS m/z: 434.3 [M+H]⁺, Calculated MW: 433.2

### Intermediate E19. Synthesis of (2S)-3-methyl-2-{methyl[7-(prop-2-enoyl)-5-oxa-2,7-diazaspiro[3.4]octan-2-yl]carbonylamino}butanoic acid

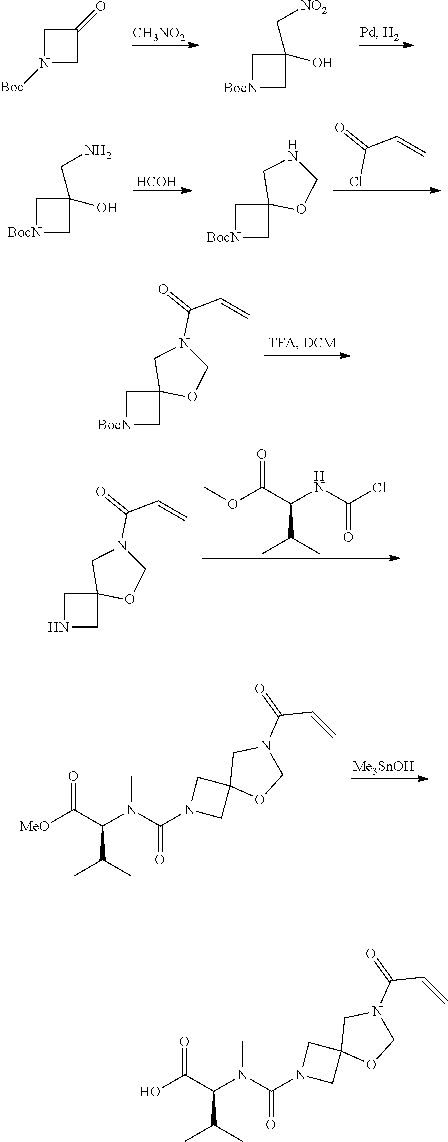

Step 1. To a solution of the tert-butyl 3-oxoazetidine-1-carboxylate (10 g, 0.058 mol, 1 eq) in EtOH (30 mL) was added CH₂NO₂ (12 mL) and triethylamine (0.59 g, 0.0058 mol, 0.1 eq). The resulting mixture was stirred for 16 h at 20° C. The mixture was concentrated under reduced pressure to afford tert-butyl 3-hydroxy-3-(nitromethyl)azetidine-1-carboxylate (13.5 g, 95% yield) as a yellow solid. ESI-MS m/z=255.1 [M+Na]⁺; Calculated MW: 232.11

Step 2. To a solution of tert-butyl 3-hydroxy-3-(nitromethyl)azetidine-1-carboxylate (13.5 g, 0.058 mol, 1.0 equiv) in MeOH (100 mL) was added Pd/C (1 g). The reaction mixture was then stirred at 20° C. for 16 hrs under hydrogen (15 psi). The resulting mixture was filtered and the filtrate was concentrated to afford tert-butyl 3-(aminomethyl)-3-hydroxyazetidine-1-carboxylate (12 g, 97% yield) as a white solid. ESI-MS m/z=103.2 [M−Boc+H]⁺; Calculated MW: 202.13

Step 3. To a solution of tert-butyl 3-(aminomethyl)-3-hydroxyazetidine-1-carboxylate (1.5 g, 7.4 mmol, 1.0 eq) in MeOH (3 mL) and NaOH(15 mL, 2 mol/L aqueous) was added HCHO (3 mL) (37 wt % in H₂O) and the reaction mixture was stirred for 16 h at 20° C. The resulting solution was extracted with DCM (3*10 mL). The combined organic layers were dried over Na₂SO₄ and concentrated under reduced pressure to afford tert-butyl 5-oxa-2,7-diazaspiro[3.4]octane-2-carboxylate (1 g, crude) as a yellow solid. The crude product was used directly in the next step. ESI-MS m/z=215.1 [M+H]⁺; Calculated MW: 214.13

Step 3. Prop-2-enoyl chloride(633 mg, 7.0 mmol, 1.5 equiv) was added to the solution of (tert-butyl 5-oxa-2,7-diazaspiro[3.4]octane-2-carboxylate (1.0 g, 4.7 mmol, 1.0 equiv) and NaHCO₃ (1.2 g, 14 mmol, 3.0 equiv) in DCM (5 mL) and H₂O (5 mL) at 0° C. The resulting mixture was stirred at 0° C. for 1 h. The mixture was then diluted with DCM (20 mL) and washed with water (20 mL) and brine (20 mL). The organic phase was collected, dried over Na₂SO₄, filtered and concentrated. The resulting residue was purified by column chromatography afford tert-butyl 7-(prop-2-enoyl)-5-oxa-2,7-diazaspiro[3.4]octane-2-carboxylate (660 mg, 50% yield) as a white solid. ESI-MS m/z=269.1 [M+H]⁺; Calculated MW: 268.14

Step 4. To a solution of the tert-butyl 7-(prop-2-enoyl)-5-oxa-2,7-diazaspiro[3.4]octane-2-carboxylate (660 mg, 2.46 mmol, 1.0 equiv) in DCM (6 mL) was added TFA (2 mL) at 20° C. The resulting solution was stirred at 20° C. for 1 h. The solvent was removed under reduced pressure to afford 1-{5-oxa-2,7-diazaspiro[3.4]octan-7-yl}prop-2-en-1-one (510 mg, crude) as a yellow solid. This crude product was used in the next step without further purification. ESI-MS m/z=169.2 [M+H]⁺; Calculated MW:168.09.

Step 5. To a solution of the methyl (2S)-3-methyl-2-(methylamino)butanoate (357 mg, 2.5 mmol, 1.0 equiv) in DCM (5 mL) was added triethylamine (1492 mg, 14.7 mmol, 6 equiv) and triphosgene (365 mg, 1.23 mmol, 0.5 equiv) at 0° C. The resulting solution was stirred at 0° C. for 1 h. The mixture was used directly in the next step.

Step 6. To a solution of methyl (2S)-2-[(chlorocarbonyl)(methyl)amino]-3-methylbutanoate (509 mg, 2.46 mmol, 1 equiv) and triethylamine (1492 mg, 14.7 mmol, 6 equiv) in DCM (15 mL) was added 1-{5-oxa-2,7-diazaspiro[3.4]octan-7-yl}prop-2-en-1-one (413 mg, 2.46 mmol, 1 equiv) at 0° C. The mixture was stirred at 0° C. for 0.5 h. The mixture was then diluted with DCM (20 mL) and washed with H₂O (30*2 mL). The organic layers were dried over Na₂SO₄ and concentrated under reduced pressure. The resulting residue was purified by chromatography to afford methyl (2S)-3-methyl-2-{methyl[7-(prop-2-enoyl)-5-oxa-2,7-diazaspiro[3.4]octan-2-yl]carbonylamino}butanoate (605 mg, 69% yield) as a white solid.

ESI-MS m/z=340.2 [M+H]⁺; Calculated MW: 339.18

Step 7. To a solution of methyl (2S)-3-methyl-2-{methyl[7-(prop-2-enoyl)-5-oxa-2,7-diazaspiro[3.4]octan-2-yl]carbonylamino}butanoate (300 mg, 0.88 mmol, 1.0 equiv) in DCE (10 mL) was added trimethyltin hydroxide (1.9 g, 10.6 mmol, 12 eq). The reaction mixture was stirred at 85° C. for 16 h. The reaction mixture was then diluted with DCM (10 mL). The resulting mixture was washed with 1 N HCl (10 mL) and the organic layers were dried over Na₂SO₄ and concentrated under reduced pressure. The resulting residue was purified by chromatography to afford (2S)-3-methyl-2-{methyl[7-(prop-2-enoyl)-5-oxa-2,7-diazaspiro[3.4]octan-2-yl]carbonylamino}butanoic acid (200 mg, 66% yield) as a white solid. ESI-MS m/z=326.1 [M+H]⁺; Calculated MW: 325.16

### Intermediate P1 and P2. Synthesis of (2S)-3-methyl-2-{methyl[3-(prop-2-enoyl)-1-oxa-3,7-diazaspiro[4.4]nonan-7-yl]carbonylamino}butanoate (P1+P2)

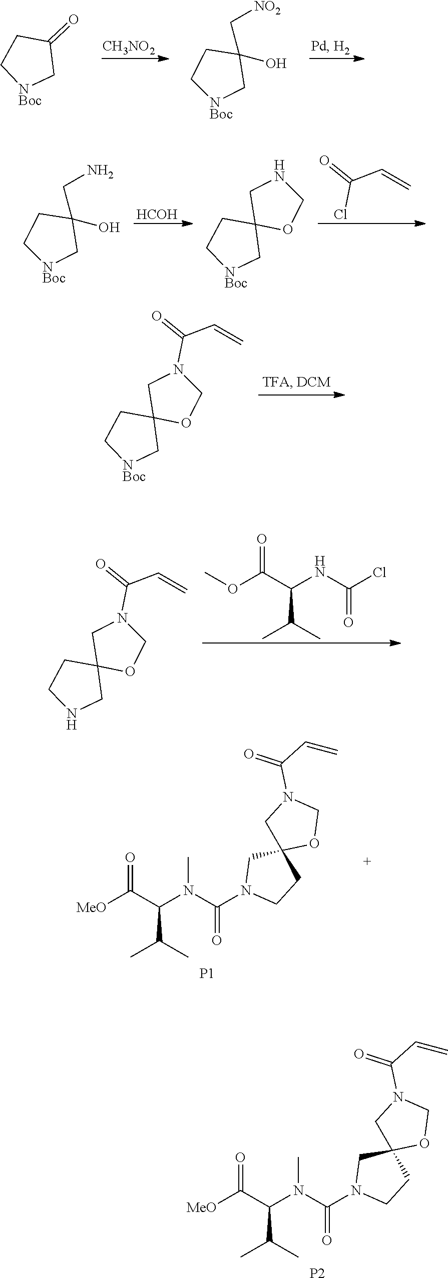

Step 1. To a solution of the tert-butyl 3-oxopyrrolidine-1-carboxylate (10 g, 0.058 mol, 1 eq) in EtOH (30 mL) was added CH₂NO₂(12 mL) and triethylamine (0.59 g, 5.8 mol, 0.1 eq). The reaction mixture was stirred for 16 h at 20° C. After which the mixture was concentrated under reduced pressure to afford tert-butyl 3-hydroxy-3-(nitromethyl)pyrrolidine-1-carboxylate (13.3 g, 80% yield) as a yellow solid.

ESI-MS m/z=269.1 [M+Na]⁺; Calculated MW: 246.12

Step 2. To a solution of tert-butyl 3-hydroxy-3-(nitromethyl)pyrrolidine-1-carboxylate (9.7 g, 0.039 mol, 1.0 equiv) in EtOH (100 mL) and THF(20 mL) was added raney Ni (2 g) and NH₃H₂O (3 mL, purity:28-30%). The resulting reaction mixture was stirred at 20° C. for 4 h under hydrogen (15 psi). The reaction mixture was filtered and the filtrate was concentrated to afford tert-butyl 3-(aminomethyl)-3-hydroxypyrrolidine-1-carboxylate (9.3 g, 87% yield) as a yellow oil. ESI-MS m/z=117.3 [M−Boc+H]⁺; Calculated MW: 216.15

Step 3. To a solution of tert-butyl 3-(aminomethyl)-3-hydroxypyrrolidine-1-carboxylate (9 g, 41.6 mmol, 1 eq) in MeOH (20 mL) and 3 N NaOH (100 mL) was added HCHO (20 mL, 37 wt % in H₂O). The reaction mixture was stirred for 16 h at 20° C. After which the resulting solution was extracted with DCM (3*100 mL) and the combined organic layers were dried over Na₂SO₄, filtered and concentrated under reduced pressure to afford tert-butyl 1-oxa-3,7-diazaspiro[4.4]nonane-7-carboxylate (6.1 g, crude) as a colorless oil. The crude product was used directly in the next step. ESI-MS m/z=129.3 [M−Boc+H]⁺; Calculated MW: 228.15

Step 4. Prop-2-enoyl chloride (3.6 g, 40 mmol, 1.5 equiv) was added to the solution of tert-butyl 1-oxa-3,7-diazaspiro[4.4]nonane-7-carboxylate (6.1 g, 26.7 mmol, 1.0 equiv) and NaHCO₃ (6.7 g, 80 mmol, 3 equiv) in DCM (60 mL) and H₂O (60 mL) at 0° C. The reaction mixture was stirred at 0° C. for 1 h. The mixture was then diluted with DCM (100 mL), and washed with water (100 mL) and brine (100 mL). The organic phase was collected, dried over Na₂SO₄, filtered and concentrated. The resulting residue was purified by chromatography to afford tert-butyl 3-(prop-2-enoyl)-1-oxa-3,7-diazaspiro[4.4]nonane-7-carboxylate (2.4 g, 30% yield) as a white solid. ESI-MS m/z=305.1 [M+Na]⁺; Calculated MW: 282.16

Step 5. To a solution of the tert-butyl 3-(prop-2-enoyl)-1-oxa-3,7-diazaspiro[4.4]nonane-7-carboxylate (2.4 g, 8.5 mmol, 1.0 equiv) in DCM (30 mL) was added TFA (10 mL) at 20° C. The reaction mixture was stirred at 20° C. for 1 h. The solvent was removed under reduced pressure to give 1-{1-oxa-3,7-diazaspiro[4.4]nonan-3-yl}prop-2-en-1-one (1.6 g, crude) as a yellow solid. The crude product was used in the next step without further purification. ESI-MS m/z=183.1 [M+H]⁺; Calculated MW:182.22

Step 6. To a solution of methyl (2S)-2-[(chlorocarbonyl)(methyl)amino]-3-methylbutanoate (1.75 g, 8.5 mmol, 1.0 equiv) and triethylamine (5131 mg, 51 mmol, 6.0 equiv) in DCM (20 mL) was added 1-{1-oxa-3,7-diazaspiro[4.4]nonan-3-yl}prop-2-en-1-one (1540 mg, 8.5 mmol, 1.0 equiv) at 0° C. The mixture was stirred at 0° C. for 0.5 h. The mixture was diluted with DCM (100 mL) and washed with H₂O (100*2 mL). The organic layers were dried over Na₂SO₄ and concentrated under reduced pressure. The resulting residue was purified by chromatography to afford methyl (2S)-3-methyl-2-{methyl[3-(prop-2-enoyl)-1-oxa-3,7-diazaspiro[4.4]nonan-7-yl]carbonylamino}butanoate (1.78 g, 56% yield) as a white solid. The desired product was separated via chiral resolution (Chromatographic columns: chiralpak-ADMobile Phase:CO₂-MeOH(0.1% DEA)) to give methyl (2S)-3-methyl-2-{methyl[(5S)-3-(prop-2-enoyl)-1-oxa-3,7-diazaspiro[4.4]nonan-7-yl]carbonylamino}butanoate(P1, 800 mg; P2, 780 mg). ESI-MS m/z=354.2 [M+H]⁺; Calculated MW: 353.20

### Intermediate P1-OH. Synthesis of (2S)-3-methyl-2-{methyl[(5R)-3-(prop-2-enoyl)-1-oxa-3,7-diazaspiro[4.4]nonan-7-yl]carbonylamino}butanoic acid

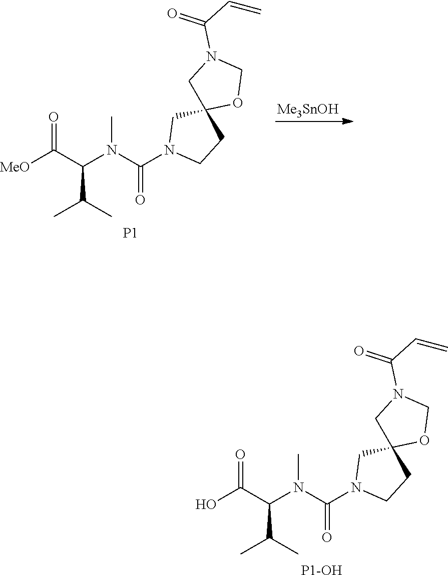

Step 1. To a solution of (P1) methyl (2S)-3-methyl-2-{methyl[(5S)-3-(prop-2-enoyl)-1-oxa-3,7-diazaspiro[4.4]nonan-7-yl]carbonylamino}butanoate (330 mg, 0.93 mmol, 1.0 equiv) in DCE (10 mL) was added trimethyltin hydroxide (2.5 g, 14 mmol, 15 eq). The reaction mixture was stirred at 85° C. for 16 h. The reaction mixture was then diluted with DCM (10 mL) and the resulting mixture was washed with 1 N HCl (10 mL). The organic layers were dried over Na₂SO₄ and concentrated under reduced pressure. The resulting residue was purified by chromatography to afford (2S)-3-methyl-2-{methyl[(5R)-3-(prop-2-enoyl)-1-oxa-3,7-diazaspiro[4.4]nonan-7-yl]carbonylamino}butanoic acid (150 mg, 45% yield) as a white solid.

ESI-MS m/z=340.2 [M+H]⁺; Calculated MW: 339.18

### Intermediate P2-OH. Synthesis of (2S)-3-methyl-2-{methyl[(5S)-3-(prop-2-enoyl)-1-oxa-3,7-diazaspiro[4.4]nonan-7-yl]carbonylamino}butanoic acid

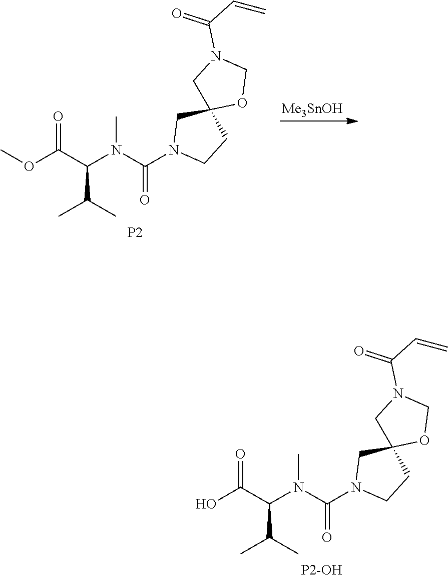

A mixture of (P2) methyl (2S)-3-methyl-2-{methyl[3-(prop-2-enoyl)-1-oxa-3,7-diazaspiro[4.4]nonan-7-yl]carbonylamino}butanoate (165 mg, 0.47 mmol, 1.0 equiv) and (Me)₃SnOH (1.7 g, 9.3 mmol, 20 equiv) in DCE (2 mL) was stirred at 85° C. for 24 h. The mixture was diluted with DCM (20 mL), and then washed with 1 N HCl (20 mL), water (15 mL) and brine (15 mL). The organic phase was collected, dried over Na₂SO₄, filtered and concentrated. The resulting residue was purified by chromatography to afford (2S)-3-methyl-2-{methyl[(5S)-3-(prop-2-enoyl)-1-oxa-3,7-diazaspiro[4.4]nonan-7-yl]carbonylamino}butanoic acid (100 mg, 60% yield) as an off-white solid. ESI-MS m/z: 340.2 [M+H]⁺. Calculated MW: 339.18

### Synthesis of 1-(4-(dimethylamino)-4-methylpent-2-ynoyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,3)-oxadiazola-1(5,3)-indola-6(1,3)-pyridazinacycloundecaphane-4-yl)amino)-3-methyl-1-oxobutan-2-yl)-4-fluoro-N-methylpiperidine-4-carboxamide

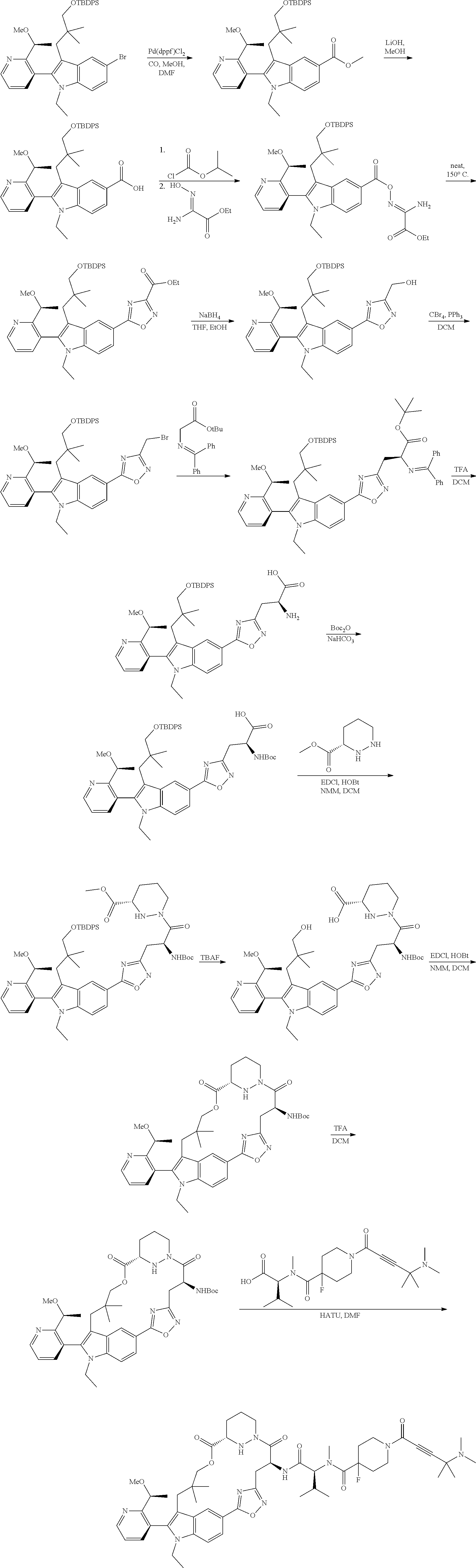

Step 1. A mixture of (2M)-5-bromo-3-[3-[(tert-butyldiphenylsilyl)oxy]-2,2-dimethylpropyl]-1-ethyl-2-[2-[(1S)-1-methoxyethyl]pyridin-3-yl]indole (10.0 g, 14.6 mmol), Pd(dppf)Cl₂. DCM (1.19 g, 1.46 mmol) and TEA (2.66 g, 26.3 mmol) in DMF (50 mL) and MeOH (1 mL) under an atmosphere of CO was heated too 100° C. and stirred overnight. H₂O (100 mL) was added, and the mixture extracted with EtOAc (3×100 mL). The combined organic layers were dried over anhydrous Na₂SO₄ and filtered. The filtrate was concentrated under reduced pressure and the residue was purified by silica gel column chromatography to give (2M)-3-[3-[(tert-butyldiphenylsilyl)oxy]-2,2-dimethylpropyl]-1-ethyl-2-[2-[(1 S)-1-methoxyethyl]pyridin-3-yl]indole-5-carboxylate (8.0 g, 74% yield) as a foam. LCMS (ESI): m/z [M+H]⁺ calc'd for C₄₁H₅₀N₂O₄Si 662.4; found 663.4.

Step 2. To a mixture of (2M)-3-[3-[(tert-butyldiphenylsilyl)oxy]-2,2-dimethylpropyl]-1-ethyl-2-[2-[(1S)-1-methoxyethyl]pyridin-3-yl]indole-5-carboxylate (3.90 g, 5.9 mmol) in THE (10 mL) and MeOH (30 mL) at 0° C. was added LiOH (0.70 g, 29.2 mmol) in H₂O (30 mL) dropwise. The mixture was warmed to rt and stirred for 3 h, then acidified to pH ˜7 with aqueous HCl and the mixture extracted with EtOAc (3×20 mL). The combined organic layers were washed with brine (2×20 mL), dried over anhydrous Na₂SO₄, filtered and the filtrate was concentrated under reduced pressure to give (2M)-3-[3-[(tert-butyldiphenylsilyl)oxy]-2,2-dimethylpropyl]-1-ethyl-2-[2-[(1S)-1-methoxyethyl]pyridin-3-yl]indole-5-carboxylic acid (2.89 g) as a solid. LCMS (ESI): m/z [M+H]⁺ calc'd for C₄₀H₄₈N₂O₄Si 648.3; found 649.3.

Step 3. To a mixture of (2M)-3-[3-[(tert-butyldiphenylsilyl)oxy]-2,2-dimethylpropyl]-1-ethyl-2-[2-[(1 S)-1-methoxyethyl]pyridin-3-yl]indole-5-carboxylic acid (2.00 g, 3.1 mmol) and K₂CO₃ (0.85 g, 6.2 mmol) in DCM (20 mL) at 0° C. was added isopropyl chloroformate (0.76 g, 6.2 mmol) dropwise. The mixture was stirred at rt for 45 min, then H₂O was added and the mixture extracted with EtOAc (3×50 mL). The combined organic layers were dried over anhydrous Na₂SO₄, filtered and the filtrate was concentrated under reduced pressure. The residue was dissolved in DCM (20 mL) and ethyl [(Z)—N-hydroxycarbamimidoyl]formate (0.81 g, 6.2 mmol) and K₂CO₃ (0.85 g, 6.2 mmol) were added. The mixture was stirred at rt for 2 h, then H₂O was added and the mixture extracted with EtOAc (3×30 mL). The combined organic layers were dried over anhydrous Na₂SO₄ and filtered. The filtrate was concentrated under reduced pressure and the residue was purified by preparative-HPLC to give ethyl [(2)-N-[(2)-(2M)-3-[3-[(tert-butyldiphenylsilyl)oxy]-2,2-dimethylpropyl]-1-ethyl-2-[2-[(1 S)-1-methoxyethyl]pyridin-3-yl]indole-5-carbonyloxy]carbamimidoyl]formate (1.23 g, 45% yield) as a solid.

LCMS (ESI): m/z [M+H]⁺ calc'd for C₄₄H₅₄N₄O₆Si 762.4; found 763.3.

Step 4. Ethyl [(2)-N-[(2)-(2M)-3-[3-[(tert-butyldiphenylsilyl)oxy]-2,2-dimethylpropyl]-1-ethyl-2-[2-[(1 S)-1-methoxyethyl]pyridin-3-yl]indole-5-carbonyloxy]carbamimidoyl]formate (1.30 g, 1.7 mmol) was heated to 150° C. and stirred for 4 h, then purified by silica gel column chromatography to give ethyl 5-[(2M)-3-[3-[(tert-butyldiphenylsilyl)oxy]-2,2-dimethylpropyl]-1-ethyl-2-[2-[(1 S)-1-methoxyethyl]pyridin-3-yl]indol-5-yl]-1,2,4-oxadiazole-3-carboxylate (600 mg, 28% yield) as a solid. LCMS (ESI): m/z [M+H]⁺ calc'd for C₄₄H₅₂N₄O₅Si 744.4; found 745.3.

Step 5. To a mixture of ethyl 5-[(2M)-3-[3-[(tert-butyldiphenylsilyl)oxy]-2,2-dimethylpropyl]-1-ethyl-2-[2-[(1 S)-1-methoxyethyl]pyridin-3-yl]indol-5-yl]-1,2,4-oxadiazole-3-carboxylate (1.1 g, 1.5 mmol) in EtOH (6 mL) and THE (6 mL) at 0° C. was added NaBH₄ (112 mg, 3.0 mmol) in portions. The mixture was stirred at rt for 1 h, then the mixture was cooled to 0° C. and saturated NH₄Cl was added and the mixture extracted with EtOAc (30 mL). The organic layer was washed with brine (20 mL), dried over anhydrous Na₂SO₄, filtered and the filtrate was concentrated under reduced pressure to give [5-[(2M)-3-[3-[(tert-butyldiphenylsilyl)oxy]-2,2-dimethylpropyl]-1-ethyl-2-[2-[(1 S)-1-methoxyethyl]pyridin-3-yl]indol-5-yl]-1,2,4-oxadiazol-3-yl]methanol (900 mg, 78% yield) as a solid. LCMS (ESI): m/z [M+H]⁺ calc'd for C₄₂H₅₀N₄O₄Si 702.4; found 703.4.

Step 6. To a mixture of [5-[(2M)-3-[3-[(tert-butyldiphenylsilyl)oxy]-2,2-dimethylpropyl]-1-ethyl-2-[2-[(1 S)-1-methoxyethyl]pyridin-3-yl]indol-5-yl]-1,2,4-oxadiazol-3-yl]methanol (900 mg, 1.3 mmol) and Ph₃P (504 mg, 1.92 mmol) in DCM (9 mL) was added CBr₄ (637 mg, 1.92 mmol). The mixture was stirred at rt for 3 h, then H₂O was added and the mixture extracted with EtOAc (10 mL). The combined organic layers were washed with brine (10 mL), dried over anhydrous Na₂SO₄ and filtered. The filtrate was concentrated under reduced pressure and the residue was purified by silica gel column chromatography to give (2M)-5-[3-(bromomethyl)-1,2,4-oxadiazol-5-yl]-3-[3-[(tert-butyldiphenylsilyl)oxy]-2,2-dimethylpropyl]-1-ethyl-2-[2-[(1 S)-1-methoxyethyl]pyridin-3-yl]indole (700 mg, 36% yield) as a solid. LCMS (ESI): m/z [M+H]⁺ calc'd for C₄₂H₄₉BrN₄O₃Si 764.3; found 765.2.

Step 7. To a mixture of (2M)-5-[3-(bromomethyl)-1,2,4-oxadiazol-5-yl]-3-[3-[(tert-butyldiphenylsilyl)oxy]-2,2-dimethylpropyl]-1-ethyl-2-[2-[(1S)-1-methoxyethyl]pyridin-3-yl]indole (1.0 g, 1.3 mmol) and tert-butyl 2-[(diphenylmethylidene)amino]acetate (579 mg, 2.0 mmol) in toluene (4.2 mL) and DCM (1.8 mL) at 0° C. was added KOH (7.0 g, 124.8 mmol) in H₂O (2 mL) and cinchonanium (158 mg, 0.26 mmol). The mixture was warmed to rt and stirred for 3 h, then H₂O was added and the mixture extracted with EtOAc (10 mL). The combined organic layers were washed with brine (5 mL), dried over anhydrous Na₂SO₄ and filtered. The filtrate was concentrated under reduced pressure and the residue was purified by silica gel column chromatography to give tert-butyl 3-[5-[(2M)-3-[3-[(tert-butyldiphenylsilyl)oxy]-2,2-dimethylpropyl]-1-ethyl-2-[2-[(1S)-1-methoxyethyl]pyridin-3-yl]indol-5-yl]-1,2,4-oxadiazol-3-yl]-2-[(diphenylmethylidene)amino]propanoate (350 mg, 25% yield) as a solid. LCMS (ESI): m/z [M+H]⁺ calc'd for C₆₁H₆₉N₅O₅Si 979.5; found 980.4.

Step 8. To a mixture of tert-butyl 3-[5-[(2M)-3-[3-[(tert-butyldiphenylsilyl)oxy]-2,2-dimethylpropyl]-1-ethyl-2-[2-[(1 S)-1-methoxyethyl]pyridin-3-yl]indol-5-yl]-1,2,4-oxadiazol-3-yl]-2-[(diphenylmethylidene)amino]propanoate (1.80 g, 1.8 mmol) in DCM (18 mL) at 0° C. was added TFA (18 mL) dropwise. The mixture was warmed to rt and stirred for 2 h, then concentrated under reduced pressure to give 2-amino-3-[5-[(2M)-3-[3-[(tert-butyldiphenylsilyl)oxy]-2,2-dimethylpropyl]-1-ethyl-2-[2-[(1 S)-1-methoxyethyl]pyridin-3-yl]indol-5-yl]-1,2,4-oxadiazol-3-yl]propanoic acid (4 g) as an oil. LCMS (ESI): m/z [M+H]⁺ calc'd for C₄₄H₅₃N₅O₅Si 759.4; found 760.2.

Step 9. To a mixture of 2-amino-3-[5-[(2M)-3-[3-[(tert-butyldiphenylsilyl)oxy]-2,2-dimethylpropyl]-1-ethyl-2-[2-[(1 S)-1-methoxyethyl]pyridin-3-yl]indol-5-yl]-1,2,4-oxadiazol-3-yl]propanoic acid (4.0 g, 5.3 mmol) and NaHCO₃(2.65 g, 30 mmol) in THE (20 mL) and H₂O (20 mL) was added Boc₂O (1.72 g, 7.9 mmol) dropwise. The mixture was stirred at rt for 2 h, then H₂O was added and the mixture was extracted with EtOAc (3×50 mL). The combined organic layers were dried over anhydrous Na₂SO₄ and filtered. The filtrate was concentrated under reduced pressure and the residue was purified by silica gel column chromatography to give 2-[(tert-butoxycarbonyl)amino]-3-[5-[(2M)-3-[3-[(tert-butyldiphenylsilyl)oxy]-2,2-dimethylpropyl]-1-ethyl-2-[2-[(1 S)-1-methoxyethyl]pyridin-3-yl]indol-5-yl]-1,2,4-oxadiazol-3-yl]propanoic acid (1.2 g, 21% yield) as a solid. LCMS (ESI): m/z [M+H]⁺ calc'd for C₄₉H₁NO₇Si 859.4; found 860.2.

Step 10. To a mixture of 2-[(tert-butoxycarbonyl)amino]-3-[5-[(2M)-3-[3-[(tert-butyldiphenylsilyl)oxy]-2,2-dimethylpropyl]-1-ethyl-2-[2-[(1 S)-1-methoxyethyl]pyridin-3-yl]indol-5-yl]-1,2,4-oxadiazol-3-yl]propanoic acid (1.00 g, 1.2 mmol), methyl (3S)-1,2-diazinane-3-carboxylate (0.34 g, 2.3 mmol), HOBT (0.08 g, 0.6 mmol) and DIPEA (1.50 g, 11.6 mmol) in DCM (10 mL) at 0° C. under an atmosphere of N₂ was added EDCI (0.33 g, 1.7 mmol) in portions. The mixture was warmed to rt and stirred for 2 h, then H₂O (50 mL) was added and the mixture extracted with EtOAc (3×50 mL). The combined organic layers were dried over anhydrous Na₂SO₄ and filtered. The filtrate was concentrated under reduced pressure and the residue was purified by silica gel column chromatography to give methyl (3S)-1-[(2S)-2-[(tert-butoxycarbonyl)amino]-3-[5-(3-[3-[(tert-butyldiphenylsilyl)oxy]-2,2-dimethylpropyl]-1-ethyl-2-[2-[(1 S)-1-methoxyethyl]pyridin-3-yl]indol-5-yl)-1,2,4-oxadiazol-3-yl]propanoyl]-1,2-diazinane-3-carboxylate (800 mg, 63% yield) as a solid. LCMS (ESI): m/z [M+H]⁺ calc'd for C₅₅H₇₁N₇O₈Si 985.5; found 986.6.

Step 11. To a mixture of methyl (3S)-1-[(2S)-2-[(tert-butoxycarbonyl)amino]-3-[5-(3-[3-[(tert-butyldiphenylsilyl)oxy]-2,2-dimethylpropyl]-1-ethyl-2-[2-[(1 S)-1-methoxyethyl]pyridin-3-yl]indol-5-yl)-1,2,4-oxadiazol-3-yl]propanoyl]-1,2-diazinane-3-carboxylate (800 mg, 0.8 mmol) in THE (5 mL) at 0° C. under an atmosphere of N₂ was added 1M TBAF in THE (5 mL) dropwise. The mixture was heated to 60° C. and stirred overnight, then H₂O (100 mL) was added and the mixture was extracted with EtOAc (3×50 mL). The combined organic layers were dried over anhydrous Na₂SO₄, filtered and the filtrate was concentrated under reduced pressure to give (3S)-1-[(2S)-2-[(tert-butoxycarbonyl)amino]-3-[5-[(2M)-1-ethyl-3-(3-hydroxy-2,2-dimethylpropyl)-2-[2-[(1 S)-1-methoxyethyl]pyridin-3-yl]indol-5-yl]-1,2,4-oxadiazol-3-yl]propanoyl]-1,2-diazinane-3-carboxylic acid (680 mg) as a solid. LCMS (ESI): m/z [M+H]⁺ calc'd for C₃₈H₅₁N₇O₈ 733.3; found 734.3.

Step 12. To a mixture of (3S)-1-[(2S)-2-[(tert-butoxycarbonyl)amino]-3-[5-[(2M)-1-ethyl-3-(3-hydroxy-2,2-dimethylpropyl)-2-[2-[(1 S)-1-methoxyethyl]pyridin-3-yl]indol-5-yl]-1,2,4-oxadiazol-3-yl]propanoyl]-1,2-diazinane-3-carboxylic acid (500 mg, 0.68 mmol), HOBT (460 mg, 3.4 mmol) and DIPEA (2.64 g, 20.4 mmol) in DCM (100 mL) at 0° C. under an atmosphere of N₂ was added EDCI (2.61 g, 13.6 mmol) in portions. The mixture was warmed to rt and stirred overnight, then concentrated under reduced pressure and the residue was purified by preparative-TLC to give tert-butyl ((6³S,4S,Z)-11-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,3)-oxadiazola-1(5,3)-indola-6(1,3)-pyridazinacycloundecaphane-4-yl)carbamate (22 mg, 18% yield) as a solid. LCMS (ESI): m/z [M+H]⁺ calc'd for C₃₈H₄₉N₇O₇ 715.4; found 716.2.

Step 13. To a mixture of tert-butyl ((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,3)-oxadiazola-1 (5,3)-indola-6(1,3)- pyridazinacycloundecaphane-4-yl)carbamate (20 mg, 0.03 mmol) in DCM (0.30 mL) at 0° C. under an atmosphere of N₂ was added TFA (0.1 mL) dropwise. The mixture was warmed to rt and stirred for 1 h, then concentrated under reduced pressure to give (6³S,4S,2)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(5,3)-oxadiazola-1(5,3)-indola-6(1,3)-pyridazinacycloundecaphane-5,7-dione (30 mg) as an oil. LCMS (ESI): m/z [M+H]⁺ calc'd for C₃₃H₄₁N₇O₅ 615.3; found 616.4.

Step 14. To a mixture of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(5,3)-oxadiazola-1(5,3)-indola-6(1,3)-pyridazinacycloundecaphane-5,7-dione (20 mg, 0.03 mmol), DIPEA (42 mg, 0.33 mmol) and (2S)-2-(1-[1-[4-(dimethylamino)-4-methylpent-2-ynoyl]-4-fluoropiperidin-4-yl]-N-methylformamido)-3-methylbutanoic acid (19 mg, 0.05 mmol) in DMF (1 mL) at 0° C. under an atmosphere of N₂ was added HATU (16 mg, 0.04 mmol) in portions. The mixture was warmed to rt and stirred for 1 h, then purified by preparative-HPLC to give 1-(4-(dimethylamino)-4-methylpent-2-ynoyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(5,3)-oxadiazola-1(5,3)-indola-6(1,3)-pyridazinacycloundecaphane-4-yl)amino)-3-methyl-1-oxobutan-2-yl)-4-fluoro-N-methylpiperidine-4-carboxamide (2.4 mg, 7% yield) as a solid. LCMS (ESI): m/z [M+H]⁺ calc'd for C₅₃H₇₁FN₁₀O₈ 994.5; found 995.4; ¹H NMR (400 MHz, DMSO-d₆) δ 8.78 (dd, J=4.7, 1.7 Hz, 1H), 8.63 (s, 1H), 8.33 (s, 1H), 7.95-7.68 (m, 3H), 7.55 (dd, J=7.7, 4.7 Hz, 1H), 5.79 (s, 1H), 5.07 (d, J=11.7 Hz, 1H), 4.62 (d, J=10.3 Hz, 1H), 4.34-4.20 (m, 7H), 3.70-3.49 (m, 3H), 3.23 (s, 3H), 3.17-3.03 (m, 5H), 2.98-2.89 (m, 3H), 2.77 (t, J=12.2 Hz, 1H), 2.46-2.41 (m, 1H), 2.20 (dd, J=10.7, 6.6 Hz, 7H), 2.15-2.03 (m, 5H), 1.81 (d, J=12.5 Hz, 1H), 1.65 (d, J=13.0 Hz, 1H), 1.53 (d, J=11.9 Hz, 1H), 1.37 (t, J=6.3 Hz, 9H), 1.03-0.86 (m, 10H), 0.88-0.80 (m, 2H), 0.80-0.74 (m, 3H).

### Synthesis of 1-(4-(dimethylamino)-4-methylpent-2-ynoyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵, 6⁶-hexahydro-1¹H-8-oxa-2(3,5)-oxadiazola-1(5,3)-indola-6(1,3)-pyridazinacycloundecaphane-4-yl)amino)-3-methyl-1-oxobutan-2-yl)-4-fluoro-N-methylpiperidine-4-carboxamide

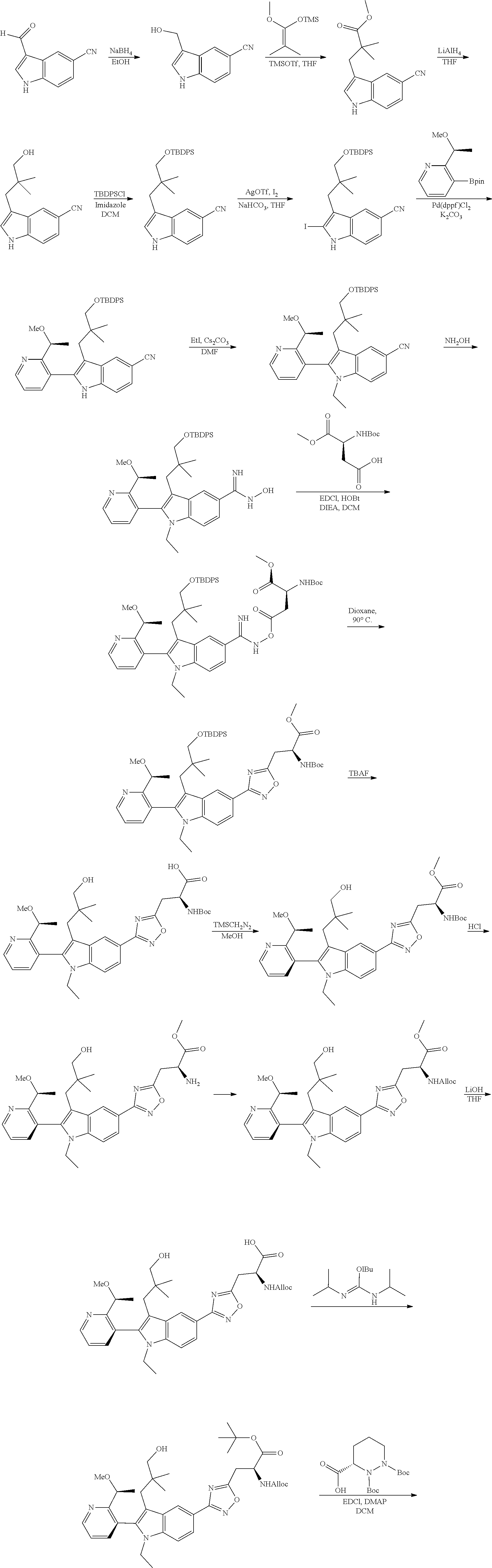

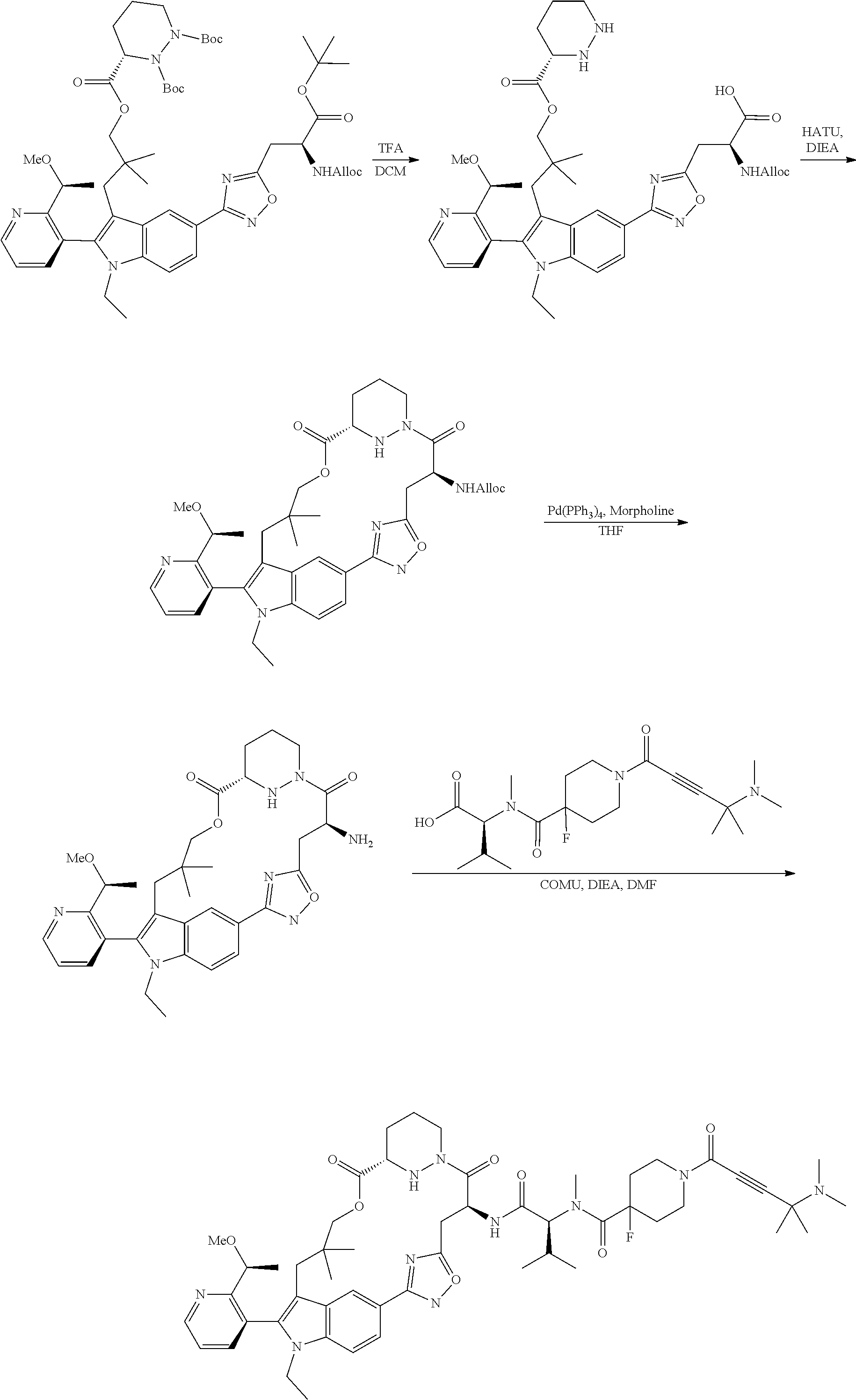

Step 1. To a mixture of 3-formyl-1H-indole-5-carbonitrile (24.8 g, 145.7 mmol) in EtOH (248 mL) at 0° C. was added NaBH₄ (8.05 g, 218.6 mmol) in portions. The mixture was stirred at 0° C. for 2 h then saturated NH₄Cl (500 mL) was added, and the volatiles were removed under reduced pressure. The mixture was extracted with DCM (3×200 mL) and the combined organic layers were washed with water (3×200 mL), dried over anhydrous Na₂SO₄ and filtered. Th filtrate was concentrated under reduced pressure and the residue was purified by silica gel column chromatography to give 3-(hydroxymethyl)-1H-indole-5-carbonitrile (21 g, 84% yield) as a solid. LCMS (ESI): m/z [M−H]⁺ calc'd for C₁₀H₈N₂O 172.1; found 171.1.

Step 2. To a mixture of 3-(hydroxymethyl)-1H-indole-5-carbonitrile (20.0 g, 116.2 mmol) in THE (200 mL) at −40° C. under an atmosphere of Ar was added [(1-methoxy-2-methylprop-1-en-1-yl)oxy]trimethylsilane (50.62 g, 290.4 mmol) and TMSOTf (19.36 g, 87.1 mmol) dropwise. The mixture was stirred at −40° C. for 2 h, then brine (200 mL) was added at 0° C. The aqueous and organic layers were partitioned and the organic layer was extracted with EtOAc (3×200 mL). The combined organic layers were concentrated under reduced pressure and the residue was purified by silica gel column chromatography to give methyl 3-(5-cyano-1H-indol-3-yl)-2,2-dimethylpropanoate (22 g, 74% yield) as a solid. LCMS (ESI): m/z [M−H]⁺ calc'd for C₁₅H₁₆N₂O₂ 256.1; found 255.1.

Step 3. To mixture of methyl 3-(5-cyano-1H-indol-3-yl)-2,2-dimethylpropanoate (22 g, 85.8 mmol) in THE (220 mL) at 0° C. was added 1M LiAlH₄ in THE (171.7 mL, 171.7 mmol) dropwise. The mixture was stirred at 0° C. for 2 h, then Na₂SO₄·10H₂O was added, the mixture was filtered and the filter cake was washed with DCM (3×300 mL). The filtrate was concentrated under reduced pressure to give 3-(3-hydroxy-2,2-dimethylpropyl)-1H-indole-5-carbonitrile (12.8 g, 65% yield) as a solid. LCMS (ESI): m/z [M−H]⁺ calc'd for C₁₄H₁₆N₂O 228.1; found 255.1.

Step 4. To a mixture of 3-(3-hydroxy-2,2-dimethylpropyl)-1H-indole-5-carbonitrile (15.0 g, 65.7 mmol) in DCM (150 mL) at 0° C. was added imidazole (11.18 g, 164.3 mmol) and TBDPSCI (23.48 g, 85.4 mmol). The mixture was warmed to rt and stirred overnight, then concentrated under reduced pressure and the residue was purified by silica gel column chromatography to give 3-[3-[(tert-butyldiphenylsilyl)oxy]-2,2-dimethylpropyl]-1H-indole-5-carbonitrile (30 g, 97% yield) as an oil. LCMS (ESI): m/z [M−H]⁺ calc'd for C₃₀H₃₄N₂OSi 466.2; found 465.2.

Step 5. To a mixture of 3-[3-[(tert-butyldiphenylsilyl)oxy]-2,2-dimethylpropyl]-1H-indole-5-carbonitrile (18.0 g, 38.6 mmol) in THE (180 mL) at 0° C. under an atmosphere of N₂ was added NaHCO₃(3.89 g, 46.3 mmol), AgOTf (10.9 g, 42.4 mmol) and 12 (8.81 g, 34.7 mmol). The mixture was stirred at 0° C. for 2 h, then 5% aqueous Na₂S₂O₃ was added and the mixture was extracted with EtOAc (3×200 mL). The combined organic layers were washed with water (3×200 mL), dried over anhydrous Na₂SO₄ and filtered. The filtrate was concentrated under reduced pressure and the residue was purified by silica gel column chromatography to give 3-[3-[(tert-butyldiphenylsilyl)oxy]-2,2-dimethylpropyl]-2-iodo-1H-indole-5-carbonitrile (18.2 g, 80% yield) as a solid. LCMS (ESI): m/z [M+Na]⁺ calc'd for C₃₀H₃₃IN₂NaOSi 615.1; found 615.0.

Step 6. To a mixture of 3-[3-[(tert-butyldiphenylsilyl)oxy]-2,2-dimethylpropyl]-2-iodo-1H-indole-5-carbonitrile (18.2 g, 30.7 mmol) and 2-[(1 S)-1-methoxyethyl]-3-(4,4,5,5-tetramethyl-1,3,2-dioxaborolan-2-yl)pyridine (32.33 g, 122.9 mmol) in 1,4-dioxane (150 mL) and H₂O (30 mL) under an atmosphere of Ar was added K₂CO₃ (10.60 g, 76.8 mmol), Pd(dppf)Cl₂ (4.49 g, 6.1 mmol). The mixture was heated to 50° C. and stirred for 3 h, then concentrated under reduced pressure and the residue was purified by silica gel column chromatography to give 3-[3-[(tert-butyldiphenylsilyl)oxy]-2,2-dimethylpropyl]-2-[2-[(1 S)-1-methoxyethyl]pyridin-3-yl]-1H-indole-5-carbonitrile (20 g) as an oil. LCMS (ESI): m/z [M+H]⁺ calc'd for C₃₈H₄₃N₃O₂Si 601.3; found 602.3.

Step 7. To a mixture of 3-[3-[(tert-butyldiphenylsilyl)oxy]-2,2-dimethylpropyl]-2-[2-[(1 S)-1-methoxyethyl]pyridin-3-yl]-1H-indole-5-carbonitrile (22.0 g, 36.6 mmol) in DMF (220 mL) at 0° C. was added Cs₂CO₃ (35.73 g, 109.7 mmol) and Etl (34.21 g, 219.3 mmol). The mixture was stirred at 0° C. for 2 h, then H₂O was added and the mixture extracted with EtOAc (300 mL). The organic layer was washed with H₂O (3×300 mL), dried over anhydrous Na₂SO₄ and filtered. The filtrate was concentrated under reduced pressure and the residue was purified by silica gel column chromatography to give 3-[3-[(tert-butyldiphenylsilyl)oxy]-2,2-dimethylpropyl]-1-ethyl-2-[2-[(1S)-1-methoxyethyl]pyridin-3-yl]indole-5-carbonitrile (15.6 g, 63% yield) as an oil. LCMS (ESI): m/z [M+H]⁺ calc'd for C₄₀H₄₇N₃O₂Si 629.3; found 630.0.

Step 8. To a mixture of 3-[3-[(tert-butyldiphenylsilyl)oxy]-2,2-dimethylpropyl]-1-ethyl-2-[2-[(1 S)-1-methoxyethyl]pyridin-3-yl]indole-5-carbonitrile (15.60 g, 24.8 mmol) in MeOH (156 mL) was added NH₂OH, 50% in H₂O (9.81 g, 296.9 mmol). The mixture was heated to 50° C. and stirred for 3 h, then concentrated under reduced pressure and the residue was purified by silica gel column chromatography to give 3-[3-[(tert-butyldiphenylsilyl)oxy]-2,2-dimethylpropyl]-1-ethyl-N-hydroxy-2-[2-[(1S)-1-methoxyethyl]pyridin-3-yl]indole-5-carboximidamide (14.6 g, 89% yield) as an oil. LCMS (ESI): m/z [M+H]⁺ calc'd for C₄₀H₅₀N₄O₃Si 662.4; found 663.2.

Step 9. To a mixture of 3-[3-[(tert-butyldiphenylsilyl)oxy]-2,2-dimethylpropyl]-1-ethyl-N-hydroxy-2-[2-[(1S)-1-methoxyethyl]pyridin-3-yl]indole-5-carboximidamide (14.60 g, 22.0 mmol) in DCM (146 mL) at −5° C. was added DIPEA (14.23 g, 110.1 mmol), HOBt (0.60 g, 4.4 mmol), followed by EDC. HCl (5.07 g, 26.4 mmol) in portions over 2 min. The mixture was allowed to warm to rt and stirred for 2 h, then concentrated under reduced pressure and the residue was purified by silica gel column chromatography to give 4-(3-[3-[(tert-butyldiphenylsilyl)oxy]-2,2-dimethylpropyl]-1-ethyl-2-[2-[(1 S)-1-methoxyethyl]pyridin-3-yl]indol-5-yl)methanimidamido 1-methyl (2S)-2-[(tert-butoxycarbonyl)amino]butanedioate (18.1 g, 92% yield) as an oil. LCMS (ESI): m/z [M+H]⁺ calc'd for C₅₀H₆₅N₅O₈Si 891.5; found 892.3.

Step 10. A mixture of 4-(3-[3-[(tert-butyldiphenylsilyl)oxy]-2,2-dimethylpropyl]-1-ethyl-2-[2-[(1 S)-1-methoxyethyl]pyridin-3-yl]indol-5-yl)methanimidamido 1-methyl (2S)-2-[(tert-butoxycarbonyl)amino]butanedioate (18 g, 20.2 mmol) in 1,4-dioxane (900 mL) was heated to 90° C. and stirred for 3 h. The mixture was concentrated under reduced pressure and the residue was purified by silica gel column chromatography to give methyl 2-[(tert-butoxycarbonyl)amino]-3-[3-(3-{3-[(tert-butyldiphenylsilyl)oxy]-2,2-dimethylpropyl}-1-ethyl-2-{2-[(1 S)-1-methoxyethyl]pyridin-3-yl}indol-5-yl)-1,2,4-oxadiazol-5-yl]propanoate (16.5 g, 94% yield) as an oil. LCMS (ESI): m/z [M+H]⁺ calc'd for C₅₀H₆₃N₅O₇Si 873.4; found 874.4.

Step 11. To a mixture of methyl 2-[(tert-butoxycarbonyl)amino]-3-[3-(3-{3-[(tert-butyldiphenylsilyl)oxy]-2,2-dimethylpropyl}-1-ethyl-2-{2-[(1S)-1-methoxyethyl]pyridin-3-yl}indol-5-yl)-1,2,4-oxadiazol-5-yl]propanoate (18 g, 20.6 mmol) in THE (180 mL) was added 1M TBAF in THE (180 mL) dropwise. The mixture was heated to 60° C. and stirred overnight, then H₂O was added and the mixture extracted with DCM (3×300 mL). The combined organic layers were washed with brine (6×300 mL), dried over anhydrous Na₂SO₄, filtered and the filtrate was concentrated under reduced pressure to give 2-[(tert-butoxycarbonyl)amino]-3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}propanoic acid (14 g) as an oil. LCMS (ESI): m/z [M−H]⁺ calc'd for C₃₃H₄₃N₅O₇ 621.3; found 620.3.

Step 12. To a mixture of 2-[(tert-butoxycarbonyl)amino]-3-{3-[(2M)-1-ethyl-3-(3-hydroxy-2,2-dimethylpropyl)-2-{2-[(1 S)-1-methoxyethyl]pyridin-3-yl}indol-5-yl]-1,2,4-oxadiazol-5-yl}propanoic acid (14 g, 22.5 mmol) in MeOH (140 mL) at 0° C. was added TMSCHN₂ (12.86 g, 112.6 mmol). The mixture was stirred at 0° C. for 2 h, then concentrated under reduced pressure and the residue was purified by silica gel column chromatography to give methyl (2R)-2-[(tert-butoxycarbonyl)amino]-3-{3-[(2M)-1-ethyl-3-(3-hydroxy-2,2-dimethylpropyl)-2-{2-[(1 S)-1-methoxyethyl]pyridin-3-yl}indol-5-yl]-1,2,4-oxadiazol-5-yl}propanoate (3.5 g, 25% yield) as an oil. LCMS (ESI): m/z [M+H]⁺ calc'd for C₃₄H₄₅N₅O₇ 635.3; found 636.4.

Step 13. To a mixture of methyl (2R)-2-[(tert-butoxycarbonyl)amino]-3-{3-[(2M)-1-ethyl-3-(3-hydroxy-2,2-dimethylpropyl)-2-{2-[(1 S)-1-methoxyethyl]pyridin-3-yl}indol-5-yl]-1,2,4-oxadiazol-5-yl}propanoate (2.0 g, 3.2 mmol) in 1,4-dioxane (20 mL) was added HCl in 1,4-dioxane (20 mL). The mixture was stirred at rt for 1 h, then concentrated under reduced pressure to give methyl (2R)-2-amino-3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}propanoate (1.5 g, 89% yield) as a solid. LCMS (ESI): m/z [M+H]⁺ calc'd for C₂₉H₃₇N₅O₅ 535.3; found 536.4.

Step 14. To a mixture of methyl (2R)-2-amino-3-{3-[(2M)-1-ethyl-3-(3-hydroxy-2,2-dimethylpropyl)-2-{2-[(1 S)-1-methoxyethyl]pyridin-3-yl}indol-5-yl]-1,2,4-oxadiazol-5-yl}propanoate (3.0 g, 5.6 mmol) in THE (30 mL) and H₂O (6 mL) at 0° C. was added NaHCO₃(1.18 g, 14.0 mmol) and allyl chlorocarbonate (1.01 g, 8.4 mmol). The mixture was stirred at 0° C. for 2 h, then concentrated under reduced pressure and the residue was purified by silica gel column chromatography to give methyl 3-{3-[(2M)-1-ethyl-3-(3-hydroxy-2,2-dimethylpropyl)-2-{2-[(1 S)-1-methoxyethyl]pyridin-3-yl}indol-5-yl]-1,2,4-oxadiazol-5-yl}-2-{[(prop-2-en-1-yloxy)carbonyl]amino}propanoate (1.5 g, 43% yield) as a solid. LCMS (ESI): m/z [M+H]⁺ calc'd for C₃₃H₄₁N₅O₇ 619.3; found 620.4.

Step 15. To a mixture of methyl 3-{3-[(2M)-1-ethyl-3-(3-hydroxy-2,2-dimethylpropyl)-2-{2-[(1 S)-1-methoxyethyl]pyridin-3-yl}indol-5-yl]-1,2,4-oxadiazol-5-yl}-2-{[(prop-2-en-1-yloxy)carbonyl]amino}propanoate (1.5 g, 2.1 mmol) in THE (15 mL) at 0° C. was added LiOH (16 mg, 6.8 mmol) in H₂O (15 mL). The mixture was stirred at 0° C. for 1 h, then acidified to pH ˜4 with aqueous HCl and extracted with DCM (3×30 mL). The combined organic layers were washed with brine (3×30 mL), dried over anhydrous Na₂SO₄, filtered and the filtrate was concentrated under reduced pressure to give (2R)-3-[3-[(2M)-1-ethyl-3-(3-hydroxy-2,2-dimethylpropyl)-2-[2-[(1S)-1-methoxyethyl]pyridin-3-yl]indol-5-yl]-1,2,4-oxadiazol-5-yl]-2-[[(prop-2-en-1-yloxy)carbonyl]amino]propanoic acid (1.46 g) as a solid. LCMS (ESI): m/z [M+H]⁺ calc'd for C₃₂H₃₉N₅O₇ 605.3; found 606.3.

Step 16. To a mixture of (2R)-3-[3-[(2M)-1-ethyl-3-(3-hydroxy-2,2-dimethylpropyl)-2-[2-[(1 S)-1-methoxyethyl]pyridin-3-yl]indol-5-yl]-1,2,4-oxadiazol-5-yl]-2-[[(prop-2-en-1-yloxy)carbonyl]amino]propanoic acid (1.46 g, 2.4 mmol) in DCM (15 mL) at 0° C. was added (2)-N,N′-diisopropyltert-butoxymethanimidamide (2.41 g, 12.1 mmol). The mixture was heated to 40° C. and stirred overnight, then H₂O was added and the mixture extracted with DCM (3×20 mL). The combined organic layers were washed with aqueous NH₄Cl (3×40 mL), dried over anhydrous Na₂SO₄, filtered and the filtrate was concentrated under reduced pressure to give tert-butyl 3-{3-[(2M)-1-ethyl-3-(3-hydroxy-2,2-dimethylpropyl)-2-{2-[(1S)-1-methoxyethyl]pyridin-3-yl}indol-5-yl]-1,2,4-oxadiazol-5-yl}-2-{[(prop-2-en-1-yloxy)carbonyl]amino}propanoate (2.3 g) as a solid. LCMS (ESI): m/z [M+H]⁺ calc'd for C₃₆H₄₇N₅O₇ 661.4; found 662.4.

Step 17. To a mixture of tert-butyl 3-{3-[(2M)-1-ethyl-3-(3-hydroxy-2,2-dimethylpropyl)-2-{2-[(1 S)-1-methoxyethyl]pyridin-3-yl}indol-5-yl]-1,2,4-oxadiazol-5-yl}-2-{[(prop-2-en-1-yloxy)carbonyl]amino}propanoate (2.30 g, 3.5 mmol) in DCM (23 mL) at −5° C. was added DMAP (85 mg, 0.7 mmol), (3S)-1,2-bis(tert-butoxycarbonyl)-1,2-diazinane-3-carboxylic acid (3.44 g, 10.4 mmol) and EDCI (0.87 g, 4.5 mmol) in portions. The mixture was warmed to rt and stirred for 2 h, then concentrated under reduced pressure and the residue was purified by silica gel column chromatography to give 3-(2-[[(2M)-5-[5-[(2R)-3-(tert-butoxy)-3-oxo-2-[[(prop-2-en-1-yloxy)carbonyl]amino]propyl]-1,2,4-oxadiazol-3-yl]-1-ethyl-2-[2-[(1S)-1-methoxyethyl]pyridin-3-yl]indol-3-yl]methyl]-2-methylpropyl)_{1,2}-di-tert-butyl (3S)-1,2-diazinane-1,2,3-tricarboxylate (2.29 g, 68% yield) as a solid. LCMS (ESI): m/z [M+H]⁺ calc'd for C₅₁H₇₁N₇O₁₂ 973.5; found 974.4.

Step 18. To a mixture of 3-(2-[[(2M)-5-[5-[(2R)-3-(tert-butoxy)-3-oxo-2-[[(prop-2-en-1-yloxy)carbonyl]amino]propyl]-1,2,4-oxadiazol-3-yl]-1-ethyl-2-[2-[(1 S)-1-methoxyethyl]pyridin-3-yl]indol-3-yl]methyl]-2-methylpropyl)1,2-di-tert-butyl (3S)-1,2-diazinane-1,2,3-tricarboxylate (2.29 g, 2.4 mmol) in DCM (30 mL) at 0° C. was added TFA (10 mL) dropwise. The mixture was stirred at 0° C. for 5 h, then concentrated under reduced pressure. The mixture was basified to pH ˜7 with saturated NaHCO₃ and extracted with DCM (3×300 mL). The combined organic layers were washed with H₂O (3×60 mL), dried over anhydrous Na₂SO₄, filtered and the filtrate was concentrated under reduced pressure to give 3-{3-[(2M)-3-{3-[(3S)-1,2-diazinane-3-carbonyloxy]-2,2-dimethylpropyl}-1-ethyl-2-{2-[(1S)-1-methoxyethyl]pyridin-3-yl}indol-5-yl]-1,2,4-oxadiazol-5-yl}-2-{[(prop-2-en-1-yloxy)carbonyl]amino}propanoic acid (1.4 g, 83% yield) as a solid. LCMS (ESI): m/z [M−H]⁺ calc'd for C₃₇H₄₇N₇O₈ 717.4; found 716.5.

Step 19. To a mixture of 3-{3-[(2M)-3-{3-[(3S)-1,2-diazinane-3-carbonyloxy]-2,2-dimethylpropyl}-1-ethyl-2-{2-[(1 S)-1-methoxyethyl]pyridin-3-yl}indol-5-yl]-1,2,4-oxadiazol-5-yl}-2-{[(prop-2-en-1-yloxy)carbonyl]amino}propanoic acid (720 mg, 1.0 mmol) in DCM (7.2 mL) at 0° C. was added DIPEA (3.89 g, 30.1 mmol) and HATU (4.58 g, 12.0 mmol). The mixture was warmed to rt and stirred overnight, then concentrated under reduced pressure and the residue was purified by silica gel column chromatography to give prop-2-en-1-yl N-[(7S,13S,19M)-21-ethyl-20-[2-[(1 S)-1-methoxyethyl]pyridin-3-yl]-17,17-dimethyl-8,14-dioxo-4,15-dioxa-3,9,21,27,28-pentaazapentacyclo[17.5.2.1\hat{ }[2,5]0.1\hat{ }[9,13]0.0\hat{ }[22,26]]octacosa-1(25),2,5(28),19,22(26),23-hexaen-7-yl]carbamate (230 mg, 33% yield) as a solid. LCMS (ESI): m/z [M−H]⁺ calc'd for C₃₇H₄₅N₇O₇ 699.3; found 699.9.

Step 20. To a mixture of allyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(3,5)-oxadiazola-1(5,3)-indola-6(1,3)-pyridazinacycloundecaphane-4-yl)carbamate (135 mg, 0.19 mmol) in THE (1.35 mL) under an atmosphere of Ar was added morpholine (50 mg, 0.58 mmol) and Pd(PPh₃)₄ (22.29 mg, 0.019 mmol). The mixture was heated to 35° C. and stirred for 4 h, then directly purified by silica gel column chromatography to give (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(3,5)-oxadiazola-1(5,3)-indola-6(1,3)-pyridazinacycloundecaphane-5,7-dione (120 mg) as a solid. LCMS (ESI): m/z [M+H]⁺ calc'd for C₃₃H₄₁N₇O₅ 615.3; found 616.4.

Step 21. To a mixture of (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(3,5)-oxadiazola-1(5,3)-indola-6(1,3)-pyridazinacycloundecaphane-5,7-dione (100 mg, 0.16 mmol) in DMF (1 mL) at 0° C. was added DIPEA (315 mg, 2.44 mmol), (2S)-2-(1-[1-[4-(dimethylamino)-4-methylpent-2-ynoyl]-4-fluoropiperidin-4-yl]-N-methylformamido)-3-methylbutanoic acid (129 mg, 0.32 mmol) and COMU (104 mg, 0.24 mmol). The mixture was stirred at 0° C. for 1 h, then purified by preparative-HPLC 1-(4-(dimethylamino)-4-methylpent-2-ynoyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(3,5)-oxadiazola-1(5,3)-indola-6(1,3)-pyridazinacycloundecaphane-4-yl)amino)-3-methyl-1-oxobutan-2-yl)-4-fluoro-N-methylpiperidine-4-carboxamide (25 mg, 15% yield) as a solid. LCMS (ESI): m/z [M−H]⁺ calc'd for C₅₃H₇₁FN₁₀O₈ 994.5; found 995.8; ¹H NMR (400 MHz, DMSO-d₆) δ 8.78 (dd, J=4.8, 1.8 Hz, 1H), 8.45 (d, J=17.0 Hz, 2H), 7.86-7.75 (m, 2H), 7.71 (d, J=8.7 Hz, 1H), 7.54 (dd, J=7.7, 4.7 Hz, 1H), 5.69 (s, 1H), 5.16 (d, J=11.8 Hz, 1H), 4.71-4.49 (m, 1H), 4.41-4.06 (m, 7H), 3.68-3.47 (m, 3H), 3.23 (s, 4H), 3.15-3.05 (m, 3H), 2.94 (d, J=11.1 Hz, 2H), 2.79-2.61 (m, 1H), 2.45-2.37 (m, 1H), 2.26-1.95 (m, 12H), 1.85-1.63 (m, 2H), 1.57-1.42 (m, 1H), 1.39-1.24 (m, 9H), 1.03-0.71 (m, 12H), 0.34 (s, 3H).

### Synthesis of (4aR,7aS)-4-acryloyl-N-((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide

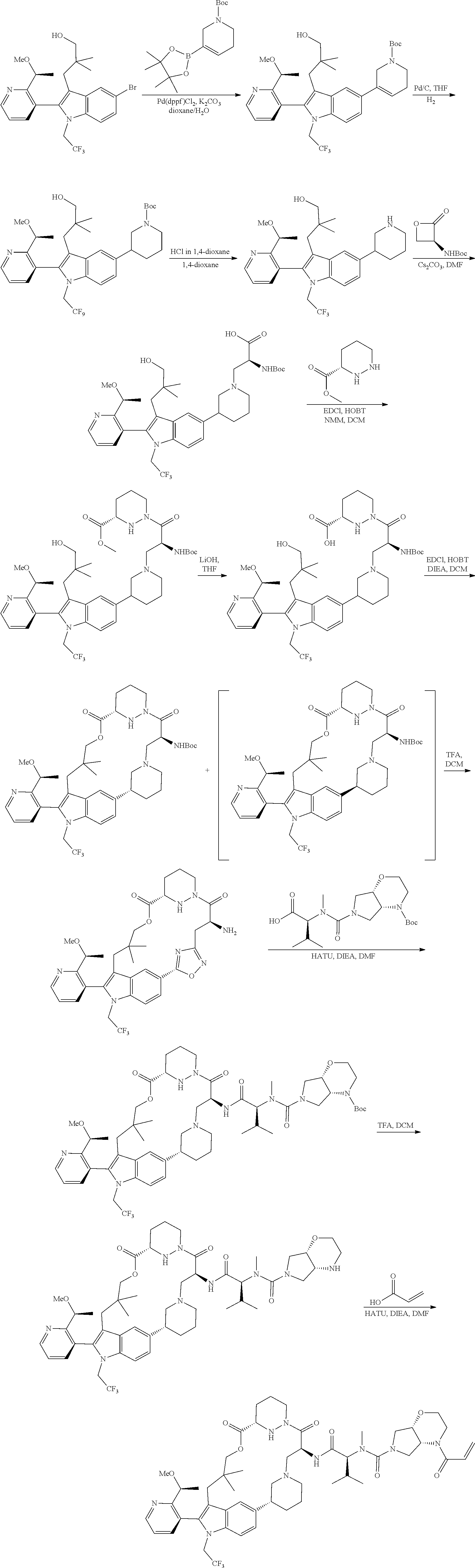

Step 1. To a mixture of 3-[(2M)-5-bromo-2-[2-[(1 S)-1-methoxyethyl] pyridin-3-yl]-1-(2,2,2-trifluoroethyl) indol-3-yl]-2,2-dimethylpropan-1-ol (10.0 g, 20.0 mmol) and tert-butyl 3-(4,4,5,5-tetramethyl-1,3,2-dioxaborolan-2-yl)-5,6-dihydro-2H-pyridine-1-carboxylate (9.29 g, 30.0 mmol) in 1,4-dioxane (85 mL) and H₂O (17 mL) under an atmosphere of N₂ was added Pd(dppf)Cl₂ (0.73 g, 1.0 mmol) and K₂CO₃ (6.92 g, 50.1 mmol) in portions. The mixture was heated to 80° C. and stirred for 3 h, then the mixture extracted with EtOAc (3×100 mL). The combined organic layers were washed with brine (3×100 mL), dried over anhydrous Na₂SO₄ and filtered. The filtrate was concentrated under reduced pressure and the residue was purified by silica gel column chromatography to give tert-butyl 3-[(2M)-3-(3-hydroxy-2,2-dimethylpropyl)-2-[2-[(1 S)-1-methoxyethyl]pyridin-3-yl]-1-(2,2,2-trifluoroethyl)indol-5-yl]-5,6-dihydro-2H-pyridine-1-carboxylate (9.0 g, 67% yield) as a solid. LCMS (ESI): m/z [M+H]⁺ calc'd for C₃₃H₄₂F₃N₃O₄ 601.3; found 602.3.

Step 2. A mixture of tert-butyl 3-[(2M)-3-(3-hydroxy-2,2-dimethylpropyl)-2-[2-[(1 S)-1-methoxyethyl]pyridin-3-yl]-1-(2,2,2-trifluoroethyl)indol-5-yl]-5,6-dihydro-2H-pyridine-1-carboxylate (6.00 g, 10.0 mmol) and Pd/C (605 mg, 5.7 mmol) in THE (60 mL) was stirred under an atmosphere of H₂ overnight. The mixture was filtered and the filtrate was concentrated under reduced pressure to give tert-butyl 3-[(2M)-3-(3-hydroxy-2,2-dimethylpropyl)-2-[2-[(1 S)-1-methoxyethyl]pyridin-3-yl]-1-(2,2,2-trifluoroethyl)indol-5-yl]piperidine-1-carboxylate (5.8 g) as an oil. LCMS (ESI): m/z [M+H]⁺ calc'd for C₃₃H₄₄F₃N₃O₄ 603.3; found 604.3.

Step 3. To a mixture of tert-butyl 3-[(2M)-3-(3-hydroxy-2,2-dimethylpropyl)-2-[2-[(1 S)-1-methoxyethyl]pyridin-3-yl]-1-(2,2,2-trifluoroethyl)indol-5-yl]piperidine-1-carboxylate (5.70 g, 9.4 mmol) in 1,4-dioxane (30 mL) at 0° C. under an atmosphere of N₂ was added HCl in 1,4-dioxane (30 mL). The mixture was stirred at 0° C. for 2 h, then aqueous NaHCO₃ was added and the mixture was extracted with EtOAc (3×20 mL). The combined organic layers were washed with brine (3×20 mL), dried over anhydrous Na₂SO₄, filtered and the filtrate was concentrated under reduced pressure to give 3-[(2M)-2-[2-[(1S)-1-methoxyethyl]pyridin-3-yl]-5-(piperidin-3-yl)-1-(2,2,2-trifluoroethyl) indol-3-yl]-2,2-dimethylpropan-1-ol (4.8 g) as an oil. LCMS (ESI): m/z [M+H]⁺ calc'd for C₂₈H₃₆F₃N₃O₂ 503.3; found 504.3.

Step 4. To a mixture of 3-[(2M)-2-[2-[(1 S)-1-methoxyethyl] pyridin-3-yl]-5-(piperidin-3-yl)-1-(2,2,2-trifluoroethyl) indol-3-yl]-2,2-dimethylpropan-1-ol (4.6 g, 9.1 mmol) in DMF (46 mL) under an atmosphere of N₂ was added tert-butyl N-[(3S)-2-oxooxetan-3-yl]carbamate (3.46 g, 18.3 mmol) and Cs₂CO₃ (7.44 g, 22.8 mmol). The mixture was heated to 40° C. and stirred for 2 h, then H₂O was added and the mixture was extracted with EtOAc (3×50 mL). The combined organic layers were washed with brine (3×50 mL), dried over anhydrous Na₂SO₄ and filtered. The filtrate was concentrated under reduced pressure and the residue was purified by preparative-HPLC to give (2S)-2-[(tert-butoxycarbonyl) amino]-3-[3-[(2M)-3-(3-hydroxy-2,2-dimethylpropyl)-2-[2-[(1 S)-1-methoxyethyl]pyridin-3-yl]-1-(2,2,2-trifluoroethyl)indol-5-yl]piperidin-1-yl]propanoic acid (2.7 g, 39% yield) as a solid. LCMS (ESI): m/z [M+H]⁺ calc'd for C₃₆H₄₉F₃N₄O₆ 690.4; found 691.1.

Step 5. To a mixture of methyl (3S)-1,2-diazinane-3-carboxylate (835 mg, 5.79 mmol) in DCM (20 mL) at 0° C. under an atmosphere of N₂ was added NMM (2.93 g, 29.0 mmol), (2S)-2-[(tert-butoxycarbonyl)amino]-3-[3-[(2M)-3-(3-hydroxy-2,2-dimethylpropyl)-2-[2-[(1 S)-1-methoxyethyl]pyridin-3-yl]-1-(2,2,2-trifluoroethyl)indol-5-yl]piperidin-1-yl] propanoic acid (2.00 g, 2.9 mmol), EDCI (833 mg, 4.3 mmol) and HOBT (196 mg, 1.5 mmol) in portions. The mixture was stirred at rt for h, then H₂O was added and the mixture extracted with DCM (3×10 mL). The combined organic layers were washed with brine (3×10 mL), dried over anhydrous Na₂SO₄ and filtered. The filtrate was concentrated under reduced pressure and the residue was purified by silica gel column chromatography to give methyl (3S)-1-[(2S)-2-[(tert-butoxycarbonyl)amino]-3-[3-[(2M)-3-(3-hydroxy-2,2-dimethylpropyl)-2-[2-[(1 S)-1-methoxyethyl]pyridin-3-yl]-1-(2,2,2-trifluoroethyl)indol-5-yl]piperidin-1-yl]propanoyl]-1,2-diazinane-3-carboxylate (2.0 g, 72% yield) as a solid. LCMS (ESI): m/z [M+H]⁺ calc'd for C₄₂H₅₉F₃N₆O₇ 816.4; found 817.5.

Step 6. To a mixture of methyl (3S)-1-[(2S)-2-[(tert-butoxycarbonyl)amino]-3-[3-[(2M)-3-(3-hydroxy-2,2-dimethylpropyl)-2-[2-[(1 S)-1-methoxyethyl]pyridin-3-yl]-1-(2,2,2-trifluoroethyl)indol-5-yl]piperidin-1-yl]propanoyl]-1,2-diazinane-3-carboxylate (2.0 g, 2.5 mmol) in THE (20 mL) under an atmosphere of N₂ was added 1M LiOH (12.24 mL, 12.24 mmol). The mixture was stirred at rt, then acidified to pH ˜6 with 1M HCl and the mixture extracted with EtOAc (3×10 mL). The combined organic layers were washed with brine (3×10 mL), dried over anhydrous Na₂SO₄, filtered and the filtrate was concentrated under reduced pressure to give (3S)-1-[(2S)-2-[(tert-butoxycarbonyl)amino]-3-[3-[(2M)-3-(3-hydroxy-2,2-dimethylpropyl)-2-[2-[(1 S)-1-methoxyethyl]pyridin-3-yl]-1-(2,2,2-trifluoroethyl)indol-5-yl]piperidin-1-yl]propanoyl]-1,2-diazinane-3-carboxylic acid (1.8 g) as a solid. LCMS (ESI): m/z [M+H]⁺ calc'd for C₄₁H₅₇F₃N₆O₇ 802.4; found 803.5.

Step 7. To a mixture of (3S)-1-[(2S)-2-[(tert-butoxycarbonyl)amino]-3-[3-[(2M)-3-(3-hydroxy-2,2-dimethylpropyl)-2-[2-[(1 S)-1-methoxyethyl]pyridin-3-yl]-1-(2,2,2-trifluoroethyl)indol-5-yl]piperidin-1-yl]propanoyl]-1,2-diazinane-3-carboxylic acid (1.80 g, 2.2 mmol) in DCM (360 mL) at 0° C. under an atmosphere of N₂ was added DIPEA (8.69 g, 67.3 mmol), HOBT (1.51 g, 11.2 mmol) and EDCI (8.60 g, 44.8 mmol) in portions. The mixture was stirred at rt for h, H₂O was added, and the mixture was extracted with DCM (3×10 mL) The combined organic layers were washed with brine (3×10 mL), dried over anhydrous Na₂SO₄ and filtered. The filtrate was concentrated under reduced pressure and the residue was purified by preparative-TLC to give two diastereomers of tert-butyl ((6³S, 4S)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)carbamate (160 mg, 9% yield) and (140 mg, 8% yield) both as solid. LCMS (ESI): m/z [M+H]⁺ calc'd for C₄₁H₅₅F₃N₆O₆ 784.4; found 785.7.

Step 8. To a mixture of tert-butyl ((6³S, 4S)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)carbamate (170 mg, 0.21 mmol) in DCM (2 mL) at 0° C. under an atmosphere of N₂ was added TFA (0.6 mL). The mixture was stirred at 0° C. for 2 h, then acidified to pH ˜8 with saturated aqueous NaHCO₃ and extracted with DCM (3×10 mL). The combined organic layers were washed with brine (3×10 mL), dried over anhydrous Na₂SO₄, filtered and the filtrate was concentrated under reduced pressure to give (2³R,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-5,7-dione (160 mg) as an oil. LCMS (ESI): m/z [M+H]⁺ calc'd for C₃₆H₄₇F₃N₆O₄ 684.4; found 685.4.

Step 9. To mixture of (2³R,6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-5,7-dione (150 mg, 0.22 mmol) in DMF (2 mL) at 0° C. under an atmosphere of N₂ was added DIPEA (283 mg, 2.2 mmol), (2S)-2-[(4aR,7aS)-4-(tert-butoxycarbonyl)-hexahydropyrrolo[3,4-b][1,4]oxazine-6-carbonyl(methyl)amino]-3-methylbutanoic acid (127 mg, 0.33 mmol) and HATU (100 mg, 0.26 mmol) in portions. The mixture was warmed to rt and stirred for 2 h, then H₂O was added and the mixture was extracted with EtOAc (3×10 mL). The combined organic layers were washed with brine (3×10 mL), dried over anhydrous Na₂SO₄ and filtered. The filtrate was concentrated under reduced pressure and the residue was purified by preparative-TLC to give tert-butyl (4aR,7aS)-6-(((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)- piperidinacycloundecaphane-4-yl)amino)-3-methyl-1-oxobutan-2-yl)(methyl)carbamoyl)hexahydropyrrolo[3,4-b][1,4]oxazine-4(4aH)-carboxylate (150 mg, 52% yield) as a solid. LCMS (ESI): m/z [M+H]⁺ calc'd for C₅₄H₇₆F₃N₉O₉ 1051.6; found 1052.5.

Step 10. To mixture of tert-butyl (4aR,7aS)-6-(((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)(methyl)carbamoyl)hexahydropyrrolo[3,4-b][1,4]oxazine-4(4aH)-carboxylate (150 mg, 0.14 mmol) in DCM (2 mL) at 0° C. under an atmosphere of N₂ was added TFA (0.70 mL). The mixture was warmed to rt and stirred for 2 h, then acidified to pH ˜8 with saturated NaHCO₃ and the mixture extracted with DCM (3×10 mL). The combined organic layers were washed with brine (3×10 mL), dried over anhydrous Na₂SO₄, filtered and the filtrate was concentrated under reduced pressure to give (4aR,7aS)-N-((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (130 mg) as an oil. LCMS (ESI): m/z [M+H]⁺ calc'd for C₄₉H₆₈F₃N₉O₇ 951.5; found 952.6.

Step 11. To a mixture of (4aR,7aS)-N-((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (120 mg, 0.13 mmol) in DMF (2 mL) at 0° C. under an atmosphere of N₂ was added DIPEA (163 mg, 1.26 mmol), acrylic acid (13.6 mg, 0.19 mmol) and HATU (57.5 mg, 0.15 mmol) in portions. The mixture was allowed to warm to rt and stirred for 2 h, then H₂O was added and the mixture extracted with EtOAc (3×10 mL). The combined organic layers were washed with brine (3×10 mL), dried over anhydrous Na₂SO₄ and filtered. The filtrate was concentrated under reduced pressure and the residue was purified by preparative-HPLC to give (4aR,7aS)-4-acryloyl-N-((2S)-1-(((2³R,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)- piperidinacycloundecaphane-4-yl)amino)-3-methyl-1-oxobutan-2-yl)-N-methylhexahydropyrrolo[3,4-b][1,4]oxazine-6(2H)-carboxamide (16 mg, 12% yield) as a solid. LCMS (ESI): m/z [M+H]⁺ calc'd for C₅₂H₇₀F₃N₉O₈ 1005.5; found 1006.9; ¹H NMR (300 MHz, DMSO-d₆) δ 8.83 (dd, J=4.7, 1.7 Hz, 1H), 7.84 (t, J=7.4 Hz, 2H), 7.71 (d, J=8.5 Hz, 1H), 7.60 (dd, J=7.8, 4.7 Hz, 1H), 7.40 (s, 1H), 7.24 (d, J=8.5 Hz, 1H), 6.94-6.79 (m, 1H), 6.25 (d, J=16.7 Hz, 1H), 5.87-5.77 (m, 2H), 5.77 (s, 1H), 5.59-5.42 (m, 1H), 5.34 (d, J=12.0 Hz, 1H), 4.83 (s, 2H), 4.31 (d, J=12.9 Hz, 1H), 4.22 (d, J=6.8 Hz, 1H), 4.10-4.01 (m, 2H), 3.93 (d, J=11.3 Hz, 5H), 3.82-3.62 (m, 4H), 3.67-3.56 (m, 4H), 3.59-3.44 (m, 5H), 3.44-3.31 (m, 1H), 3.23 (d, J=5.7 Hz, 4H), 3.09 (s, 1H), 2.88-2.69 (m, 7H), 2.73-2.59 (m, 3H), 2.35 (m, 2H), 2.29 (s, 1H), 2.12 (s, 4H), 2.06 (s, 1H), 1.84 (s, 1H), 1.74-1.56 (m, 4H), 1.45 (d, J=6.1 Hz, 3H), 1.35-1.04 (m, 1H), 1.05-0.91 (m, 2H), 0.92-0.63 (m, 8H), 0.43 (s, 3H).

### Synthesis of (4aR,7aS)-4-acryloyl-N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide

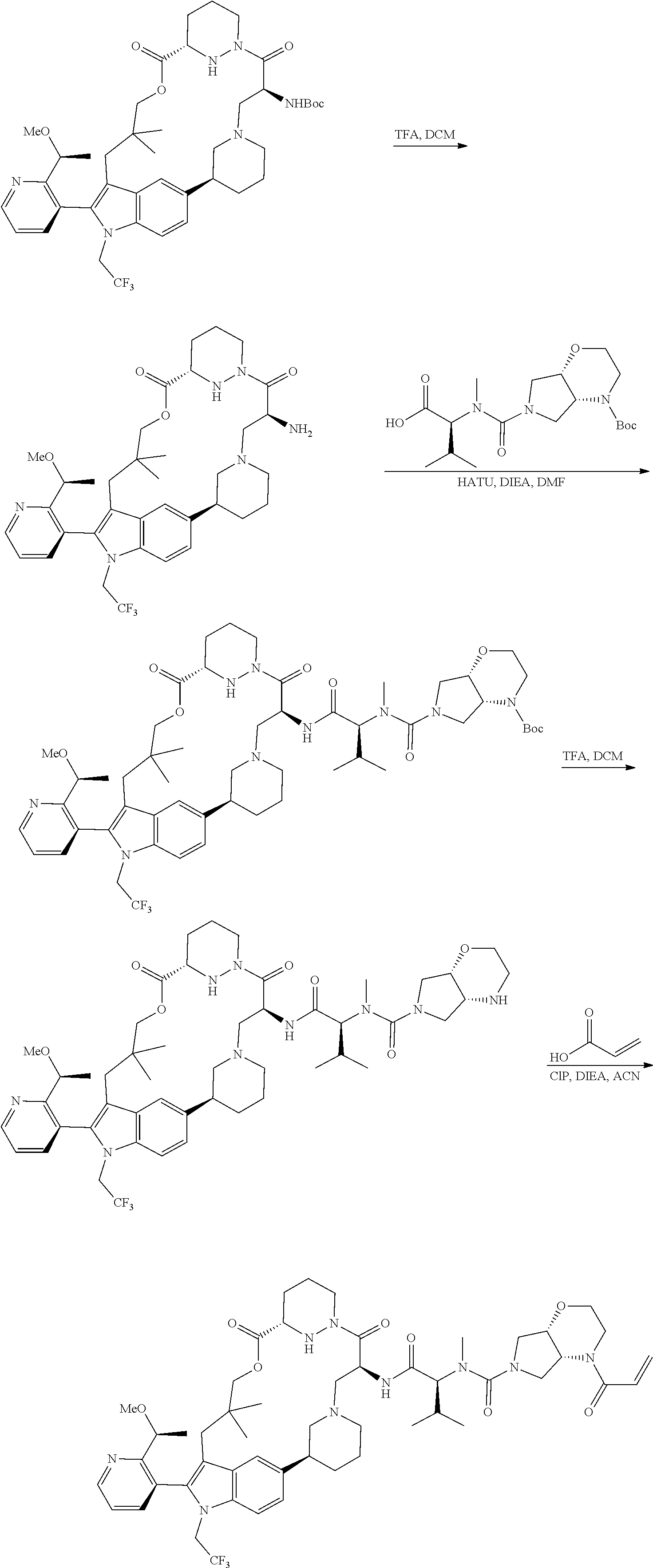

Step 1. To a mixture of tert-butyl ((2³S,6³S,4S)-1²-(2-((S)-1-methoxyethyl) pyridin-3-yl)-10,10-dimethyl-5,7-dioxo-1¹-(2,2,2-trifluoroethyl)-6¹,6²,6³,6⁴,6⁵,6⁶-hexahydro-1H-8-oxa-1(5,3)-indola-6(1,3)-pyridazina-2(3,1)-piperidinacycloundecaphane-4-yl) carbamate (200 mg, 0.26 mmol) in DCM (2 mL) at 0° C. was added TEA (0.7 mL). The mixture was stirred at 0° C. for 2 h, then acidified to pH ˜8 with saturated NaHCO₃ and extracted with DCM (3×10 mL). The combined organic layers were washed with brine (3×10 mL), dried over anhydrous Na₂SO₄, filtered and the filtrate was concentrated under reduced pressure to give (2³S,6³S,4S)-4-amino-1²-(2-((S)-1-methoxyethyl) pyridin-3-yl)-10,10-dimethyl-1¹-(2,2,2-trifluoroethyl)-6¹,6²,6³,6⁴,6⁵,6⁶-hexahydro-1¹H-8-oxa-1 (5,3)-indola-6(1,3)-pyridazina-2(3,1)-piperidinacycloundecaphane-5,7-dione (200 mg) as an oil. LCMS (ESI): m/z [M+H]⁺ calc'd for C₃₆H₄₇F₃N₆O₄ 684.4; found 985.4.

Step 2. To a mixture of (2³S,6³S,4S)-4-amino-1²-(2-((S)-1-methoxyethyl) pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-5,7-dione (200 mg, 0.29 mmol) in DMF (2 mL) at 0° C. under an atmosphere of N₂ was added DIPEA (378 mg, 2.9 mmol), (2S)-2-[(4aR,7aS)-4-(tert-butoxycarbonyl)-hexahydropyrrolo[3,4-b] [1,4] oxazine-6-carbonyl (methyl) amino]-3-methylbutanoic acid (169 mg, 0.44 mmol) and HATU (133 mg, 0.35 mmol). The mixture was warmed to rt and stirred for 2 h, then H₂O added and the mixture was extracted with EtOAc (3×10 mL). The combined organic layers were washed with brine (3×10 mL), dried over anhydrous Na₂SO₄ and filtered. The filtrate was concentrated under reduced pressure and the crude residue was purified by preparative-TLC to give tert-butyl (4aR,7aS)-6-(((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)(methyl) carbamoyl)hexahydropyrrolo[3,4-b][1,4]oxazine-4(4aH)-carboxylate (230 mg, 67% yield) as a solid. LCMS (ESI): m/z [M+H]⁺ calc'd for C₅₄H₇₆F₃N₉O₉ 1051.6; found 1052.6.

Step 3. To a mixture of tert-butyl (4aR,7aS)-6-(((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)(methyl) carbamoyl)hexahydropyrrolo[3,4-b][1,4]oxazine-4(4aH)-carboxylate (230 mg, 0.22 mmol) in DCM (3 mL) at 0° C. under an atmosphere of N₂ was added TFA. The mixture was warmed to rt and stirred for 2 h, then H₂O added and the mixture extracted with DCM (3×10 mL). The combined organic layers were washed with brine (3×10 mL), dried over anhydrous Na₂SO₄, filtered and the filtrate was concentrated under reduced pressure to give (4aR,7aS)-N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (220 mg) as a solid.

LCMS (ESI): m/z [M+H]⁺ calc'd for C₄₉H₆₈F₃N₉O₇ 951.5; found 952.5.

Step 4. To a mixture of (4aR,7aS)-N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (220 mg, 0.23 mmol) in ACN (3 mL) at 0° C. under an atmosphere of N₂ was added DIPEA (299 mg, 2.3 mmol), acrylic acid (25 mg, 0.35 mmol) and CIP (77 mg, 0.28 mmol). The mixture was warmed to rt and stirred for 2 h, then H₂O added and the mixture extracted with EtOAc (3×10 mL). The combined organic layers were washed with brine (3×10 mL), dried over anhydrous Na₂SO₄ and filtered. The filtrate was concentrated under reduced pressure and the crude residue was purified by preparative-HPLC to give (4aR,7aS)-4-acryloyl-N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-piperidinacycloundecaphane-4-yl)amino)-3-methyl-1-oxobutan-2-yl)-N-methylhexahydropyrrolo[3,4-b][1,4]oxazine-6(2H)-carboxamide (20 mg, 8% yield) as a solid. LCMS (ESI): m/z [M+H]⁺ calc'd for C₅₂H₇₀F₃N₉O₈ 1005.5; found 1006.9; ¹H NMR (300 MHz, DMSO-d₆) δ 8.75 (dd, J=4.7, 1.8 Hz, 1H), 7.77 (d, J=7.9 Hz, 1H), 7.67 (t, J=9.2 Hz, 2H), 7.58-7.48 (m, 2H), 7.17 (d, J=8.6 Hz, 1H), 6.86 (dd, J=17.2, 10.6 Hz, 1H), 6.20 (d, J=16.5 Hz, 1H), 5.80-5.59 (m, 2H), 5.48 (s, 1H), 5.11 (d, J=11.7 Hz, 1H), 4.73 (d, J=15.3 Hz, 2H), 4.35 (d, J=12.8 Hz, 1H), 4.21-4.04 (m, 2H), 3.99-3.71 (m, 6H), 3.67-3.49 (m, 3H), 3.30-3.05 (m, 7H), 3.04-2.91 (m, 3H), 2.77-2.60 (m, 9H), 2.09 (d, J=42.2 Hz, 5H), 1.81 (d, J=28.6 Hz, 2H), 1.64-1.56 (m, 5H), 1.40 (d, J=6.1 Hz, 3H), 0.95 (s, 3H), 0.82 (t, J=6.4 Hz, 6H), 0.21 (s, 3H).

### Synthesis of 4-acryloyl-N-((2S)-1-(((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)-pyridazinacycloundecaphane-4-yl)amino)-3-methyl-1-oxobutan-2-yl)-N-methyl-1-oxa-4,9-diazaspiro[5.5]undecane-9-carboxamide

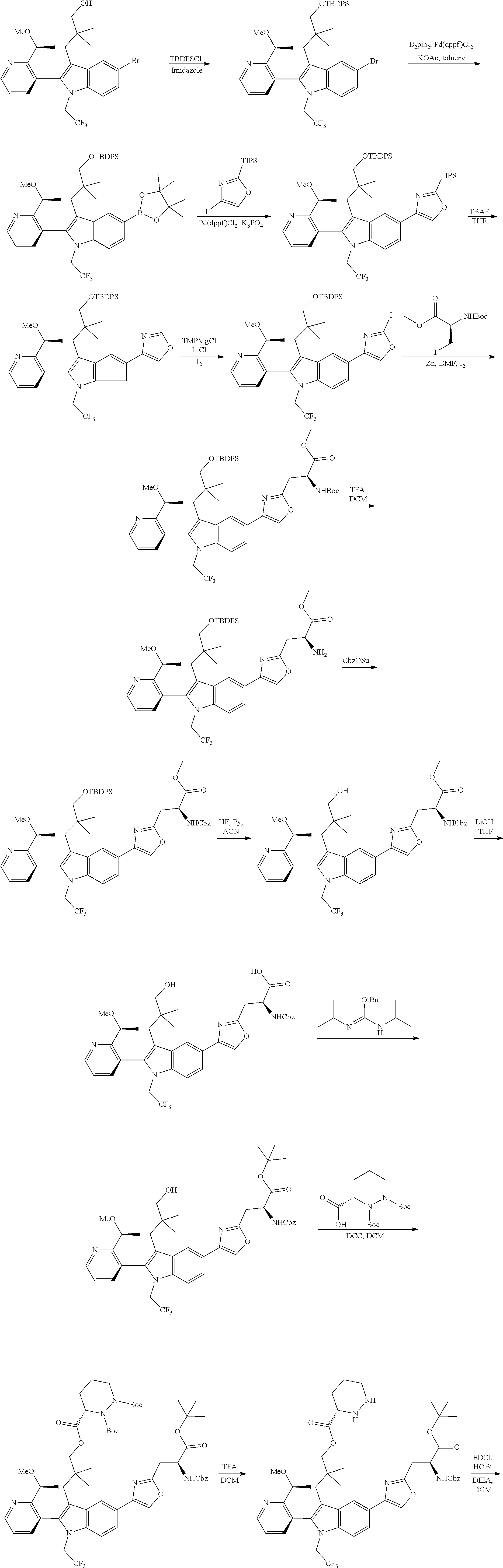

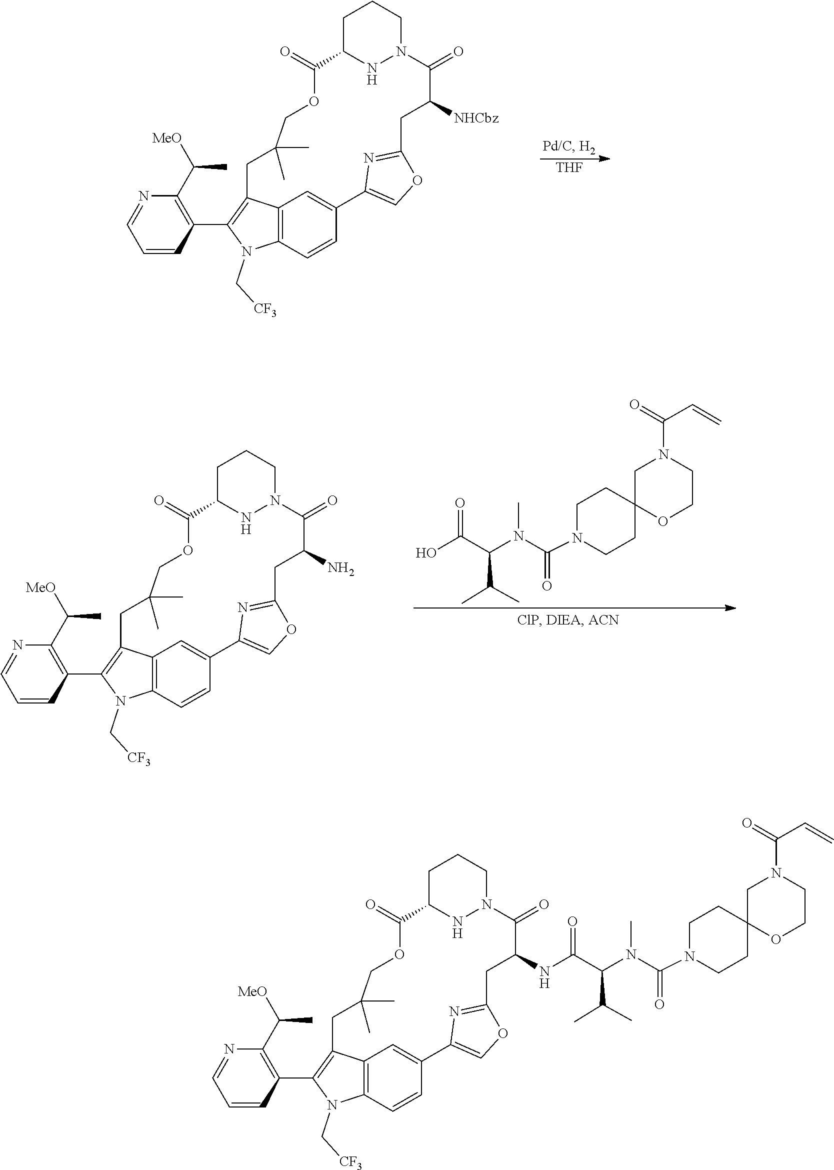

Step 1. To a mixture of (S)-3-(3-((tert-butyldiphenylsilyl)oxy)-2,2-dimethyl propyl)-2-(2-(1-methoxyethyl)pyridin-3-yl)-5-(4,4,5,5-tetramethyl-1,3,2-dioxaborolan-2-yl)-1-(2,2,2-trifluoroethyl)-1H-indole (6.3 g, 8.0 mmol) and 4-iodo-2-(triisopropylsilyl)-1,3-oxazole (8.46 g, 24.1 mmol) in 1,4-dioxane (60 mL) and H₂O (12 mL) under an atmosphere of Ar was added K₃PO₄ (4.26 g, 20.1 mmol) and Pd(dppf)Cl₂ (0.59 g, 0.80 mmol). The mixture was heated to 70° C. and stirred for 2 h, then concentrated under reduced pressure and the residue was purified by silica gel column chromatography to give (S)-4-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-2-(triisopropylsilyl)oxazole (8.84 g) as an oil. LCMS (ESI): m/z [M+H]⁺ calc'd for C₅₁H₆₆F₃N₃O₃Si₂ 881.5; found 882.5.

Step 2. To a mixture of (2M)-3-[3-[(tert-butyldiphenylsilyl)oxy]-2,2-dimethylpropyl]-2-[2-[(1 S)-1-methoxyethyl]pyridin-3-yl]-1-(2,2,2-trifluoroethyl)-5-[2-(triisopropylsilyl)-1,3-oxazol-4-yl]indole (8.84 g, 10.0 mmol) in THE (90 mL) at 0° C. was added 1M TBAF in THE (10.0 mL, 10.0 mmol). The mixture was stirred at 0° C. for 1 h, then washed with saturated NH₄Cl (3×100 mL). The combined aqueous layers were extracted with EtOAc (3×100 mL) and the combined organic layers were dried over anhydrous Na₂SO₄ and filtered. The filtrate was concentrated under reduced pressure to give (2M)-3-[3-[(tert-butyldiphenylsilyl)oxy]-2,2-dimethylpropyl]-2-[2-[(1 S)-1-methoxyethyl]pyridin-3-yl]-5-(1,3-oxazol-4-yl)-1-(2,2,2-trifluoroethyl)indole (8.4 g) as an oil. LCMS (ESI): m/z [M+H]⁺ calc'd for C₄₂H₄₆F₃N₃O₃Si 725.3; found 726.4.

Step 3. To a mixture of (2M)-3-[3-[(tert-butyldiphenylsilyl)oxy]-2,2-dimethylpropyl]-2-[2-[(1 S)-1-methoxyethyl]pyridin-3-yl]-5-(1,3-oxazol-4-yl)-1-(2,2,2-trifluoroethyl)indole (4.5 g, 6.2 mmol) in THE (45 mL) at 0° C. under an atmosphere of N₂ was added 1M TMPMgCl·LiCl (12.2 mL, 12.2 mmol) dropwise. The mixture was warmed to rt and stirred for 1 h, then a mixture of 12 (1.89 g, 7.4 mmol) in THE (10 mL) was added dropwise. The mixture was stirred at rt for 1 h, then re-cooled to 0° C. and saturated NH₄Cl added and the mixture extracted with EtOAc (3×10 mL). The combined organic layers were washed with brine (2×10 mL), dried over anhydrous Na₂SO₄ and filtered. The filtrate was concentrated under reduced pressure and the residue was purified by preparative-TLC to give (S)-4-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-2-iodooxazole (3.0 g, 57% yield) as a solid. LCMS (ESI): m/z [M+H]⁺ calc'd for C₄₂H₄₅F31N₃₀₃Si 851.2; found 852.3.

Step 4. To a mixture of Zn (645 mg, 9.9 mmol) in DMF (10 mL) under an atmosphere of Ar was added 12 (125 mg, 0.49 mmol). The mixture was heated to 45° C. and stirred for 30 min, then methyl (2R)-2-[(tert-butoxycarbonyl)amino]-3-iodopropanoate (1.22 g, 3.7 mmol) in DMF (5 mL) was added dropwise at 45° C. The mixture was stirred at 45° C. for 2 h then cooled to 0° C. and (S)-4-(3-(3-((tert-butyldiphenylsilyl)oxy)-2,2-dimethylpropyl)-2-(2-(1-methoxyethyl)pyridin-3-yl)-1-(2,2,2-trifluoroethyl)-1H-indol-5-yl)-2-iodooxazole (2.1 g, 2.5 mmol), then Pd(PPh₃)₂Cl₂ (173 mg, 0.25 mmol) in DMF (20 mL) added dropwise. The mixture was heated to 75° C. and stirred for 2 h, then brine (20 mL) added and the mixture extracted with EtOAc (3×50 mL). The combined organic layers were washed with brine (3×10 mL), dried over anhydrous Na₂SO₄ and filtered. The filtrate was concentrated under reduced pressure and the residue was purified by silica gel column chromatography to give methyl (S)-2-((tert-butoxycarbonyl)amino)-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (1.6 g, 70% yield) as an oil. LCMS (ESI): m/z [M+H]⁺ calc'd for C₅₁H₆₁F₃N₄O₇Si 926.4; found 927.5.

Step 5. To a mixture of (S)-2-((tert-butoxycarbonyl)amino)-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (2.4 g, 2.6 mmol) in DCM (1.8 mL) at 0° C. was added TFA (0.6 mL). The mixture was stirred at 0° C. for 1 h, then saturated NaHCO₃ was added and the mixture was extracted with DCM/MeOH (10:1; 3×50 mL). The combined organic layers were washed with brine (3×20 mL), dried over anhydrous Na₂SO₄, filtered and the filtrate was concentrated under reduced pressure to give methyl (S)-2-amino-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (2.1 g, 98% yield) as a solid. LCMS (ESI): m/z [M+H]⁺ calc'd for C₄₆H₅₃F₃N₄O₅Si 826.4; found 827.5.

Step 6. To a mixture of (S)-2-amino-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (2.1 g, 2.5 mmol) in THE (15 mL) and H₂O (5 mL) at 0° C. was added NaHCO₃(0.64 g, 7.6 mmol) and benzyl 2,5-dioxopyrrolidin-1-yl carbonate (0.95 g, 3.8 mmol). The mixture was stirred at 0° C. for 1 h then EtOAc (20 mL) added and the mixture was washed with brine (3×10 mL). The organic layer was concentrated under reduced pressure and the residue was purified by silica gel column chromatography to give methyl (S)-2-(((benzyloxy)carbonyl)amino)-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (2.2 g, 90% yield) as a solid. LCMS (ESI): m/z [M+H]⁺ calc'd for C₅₄H₅₉EF₃N₄O₇Si 960.4; found 961.4.

Step 7. To a mixture of methyl (S)-2-(((benzyloxy)carbonyl)amino)-3-(4-(3-(3-((tert-butyldiphenylsilyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (2.2 g, 2.3 mmol) in ACN (11 mL) at 0° C. was added HF-pyridine (11 mL, 122 mmol). The mixture was warmed to rt and stirred for 1 h, then basified to pH ˜7 with saturated NaHCO₃. The aqueous and organic layers were partitioned and the organic layer was concentrated under reduced pressure to give methyl (S)-2-(((benzyloxy)carbonyl)amino)-3-(4-(3-(3-hydroxy-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (1.7 g) as a solid. LCMS (ESI): m/z [M+H]⁺ calc'd for C₃₈H₄₁F₃N₄O₇ 722.3; found 723.3.

Step 8. To a mixture of methyl (S)-2-(((benzyloxy)carbonyl)amino)-3-(4-(3-(3-hydroxy-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (1.7 g, 2.4 mmol) in THE (1.2 mL) and H₂O (0.4 mL) at 0° C. was added LiOH (0.08 g, 3.5 mmol). The mixture was stirred at 0° C. overnight, then acidified to pH ˜4 with aqueous HCl. The mixture was extracted with DCM/MeOH (10:1; 3×20 mL). The combined organic layers were dried over anhydrous Na₂SO₄, filtered and the filtrate was concentrated under reduced pressure to give (2S)-2-{[(benzyloxy)carbonyl]amino}-3-{4-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]-1,3-oxazol-2-yl}propanoic acid (1.5 g, 90% yield) as an oil. LCMS (ESI): m/z [M+H]⁺ calc'd for C₃₇H₃₉F₃N₄O₇ 708.3; found 709.3.

Step 9. To a mixture of (2S)-2-{[(benzyloxy)carbonyl]amino}-3-{4-[(2M)-3-(3-hydroxy-2,2-dimethylpropyl)-2-{2-[(1 S)-1-methoxyethyl]pyridin-3-yl}-1-(2,2,2-trifluoroethyl)indol-5-yl]-1,3-oxazol-2-yl}propanoic acid (1.5 g, 2.1 mmol) in DCM (15 mL) and (2)-N,N′-diisopropyl tert-butoxymethanimidamide (2.12 mL, 10.6 mmol). The mixture was heated to 40° C. and stirred for 3 h, then concentrated under reduced pressure and the residue was purified by silica gel column chromatography to give tert-butyl (S)-2-(((benzyloxy)carbonyl)amino)-3-(4-(3-(3-hydroxy-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (1.6 g, 99% yield) as a solid. LCMS (ESI): m/z [M+H]⁺ calc'd for C₄₁H₄₇F₃N₄O₇ 764.3; found 765.3.

Step 10. To a mixture of tert-butyl (S)-2-(((benzyloxy)carbonyl)amino)-3-(4-(3-(3-hydroxy-2,2-dimethylpropyl)-2-(2-((S)-1-methoxyethyl)pyridin-3-yl)-1-(2,2,2-trifluoroethyl)-1H-indol-5-yl)oxazol-2-yl)propanoate (1.8 g, 2.4 mmol) in DCM (16 mL) at 0° C. was added (3S)-1,2-bis(tert-butoxycarbonyl)-1,2-diazinane-3-carboxylic acid (1.04 g, 3.1 mmol) and DCC (0.65 g, 3.1 mmol). The mixture was stirred at 0° C. for 1 h, then concentrated under reduced pressure and the residue was purified by silica gel column chromatography to give 3-(3-(5-(2-((S)-2-(((benzyloxy)carbonyl)amino)-3-(tert-butoxy)-3-oxopropyl)oxazol-4-yl)-2-(2-((S)-1-methoxyethyl)pyridin-3-yl)-1-(2,2,2-trifluoroethyl)-1H-indol-3-yl)-2,2-dimethylpropyl) 1,2-di-tert-butyl (S)-tetrahydropyridazine-1,2,3-tricarboxylate (1.8 g, 80% yield) as a solid.

LCMS (ESI): m/z [M+H]⁺ calc'd for C₅₆H₇₁F₃N₆O₁₂ 1076.5; found 1077.4.

Step 11. To a mixture of 3-(3-(5-(2-((S)-2-(((benzyloxy)carbonyl)amino)-3-(tert-butoxy)-3-oxopropyl)oxazol-4-yl)-2-(2-((S)-1-methoxyethyl)pyridin-3-yl)-1-(2,2,2-trifluoroethyl)-1H-indol-3-yl)-2,2-dimethylpropyl) 1,2-di-tert-butyl (S)-tetrahydropyridazine-1,2,3-tricarboxylate (1.8 g, 1.7 mmol) in DCM (15 mL) at 0° C. was added TFA (5 mL). The mixture was stirred at 0° C. for 1 h, then saturated NaHCO₃ was added and the mixture extracted with EtOAc (3×100 mL). The combined organic layers were washed with brine (3×10 mL), dried over anhydrous Na₂SO₄, filtered and the filtrate was concentrated under reduced pressure to give (S)-2-(((benzyloxy)carbonyl)amino)-3-(4-(3-(3-(((S)-hexahydropyridazine-3-carbonyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoic acid (1.27 g, 93% yield) as a solid. LCMS (ESI): m/z [M+H]⁺ calc'd for C₄₂H₄₇F₃N₆O₈ 820.3; found 821.4.

Step 12. To a mixture of (S)-2-(((benzyloxy)carbonyl)amino)-3-(4-(3-(3-(((S)-hexahydropyridazine-3-carbonyl)oxy)-2,2-dimethylpropyl)-2-(2-((S)-1-methoxyethyl)pyridin-3-yl)-1-(2,2,2-trifluoroethyl)-1H-indol-5-yl)oxazol-2-yl)propanoic acid (870 mg, 1.1 mmol) and DIPEA (4.1 g, 31.8 mmol) in DCM (175 mL) at 0° C. was added HOBt (1.15 g, 8.5 mmol) and EDCI (8.13 g, 42.4 mmol) in portions over 15 min. The mixture was allowed to warm to rt and stirred overnight then concentrated under reduced pressure and the residue was purified by silica gel column chromatography to give benzyl ((6³S,4S,2)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)-pyridazinacycloundecaphane-4-yl)carbamate (180 mg, 21% yield) as a solid. LCMS (ESI): m/z [M+H]⁺ calc'd for C₄₂H₄₅F₃N₆O₇ 802.3; found 803.4.

Step 13. A mixture of benzyl ((6³S,4S,2)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)-pyridazinacycloundecaphane-4-yl)carbamate (150 mg, 0.19 mmol) and 10% Pd/C (0.1 g) in THE (2 mL) was stirred at 35° C. under an atmosphere of H₂ (balloon) for 1 h. The mixture was filtered through a pad of Celite and the filtrate was concentrated under reduced pressure to give (6³S,4S,Z)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-oxazola-1 (5,3)-indola-6(1,3)-pyridazinacycloundecaphane-5,7-dione (112 mg, 90% yield) as a solid. LCMS (ESI): m/z [M+H]⁺ calc'd for C₃₄H₃₉F₃N₆O₅ 668.3; found 669.3.

Step 14. To a mixture of (6³S,4S,Z)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)-pyridazinacycloundecaphane-5,7-dione (91 mg, 0.14 mmol) in ACN (1 mL) at 0° C. was added DIPEA (352 mg, 2.7 mmol) and (2S)-3-methyl-2-[methyl(4-(prop-2-enoyl)-1-oxa-4,9-diazaspiro[5.5]undecane-9-carbonyl)amino]butanoic acid (75 mg, 0.20 mmol) and 2-chloro-1,3-dimethyl-4,5-dihydro-1H-imidazol-3-ium; hexafluorophosphate(V) (46 mg, 0.16 mmol). The mixture was stirred at 0° C. for 1 h, then concentrated under reduced pressure and the residue was purified by preparative-HPLC to give 4-acryloyl-N-((2S)-1-(((6³S,4S,Z)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-oxazola-1(5,3)-indola-6(1,3)- pyridazinacycloundecaphane-4-yl)amino)-3-methyl-1-oxobutan-2-yl)-N-methyl-1-oxa-4,9-diazaspiro[5.5]undecane-9-carboxamide (29.6 mg, 21% yield) as a solid. LCMS (ESI): m/z [M+H]⁺ calc'd for C₅₂H₆₆F₃NOs 1017.5; found 1018.7; ¹H NMR (400 MHz, DMSO-d₆) δ 8.77 (dd, J=4.7, 1.8 Hz, 1H), 8.45-8.21 (m, 3H), 7.94-7.70 (m, 2H), 7.63 (d, J=7.6 Hz, 1H), 7.54 (m, 1H), 6.84 (t, J=13.8 Hz, 1H), 6.16 (d, J=16.5 Hz, 1H), 5.70 (d, J=10.5 Hz, 1H), 5.62-5.50 (m, 2H), 5.08 (d, J=11.9 Hz, 1H), 4.94-4.75 (m, 1H), 4.35 (td, J=12.1, 3.2 Hz, 1H), 4.34-4.15 (m, 2H), 3.94-3.80 (m, 1H), 3.65 (d, J=5.0 Hz, 2H), 3.57-3.48 (m, 6H), 3.28 (s, 4H), 3.19-2.93 (m, 4H), 2.93-2.62 (m, 5H), 2.40 (d, J=14.4 Hz, 1H), 2.20-2.04 (m, 2H), 1.86-1.57 (m, 5H), 1.58-1.40 (m, 2H), 1.37 (d, J=6.1 Hz, 3H), 0.98-0.77 (m, 9H), 0.28 (s, 3H).

### Synthesis of 4-acryloyl-N-((2S)-1-(((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-azetidinacycloundecaphane-4-yl)amino)-3-methyl-1-oxobutan-2-yl)-N-methyl-1-oxa-4,9-diazaspiro[5.5]undecane-9-carboxamide

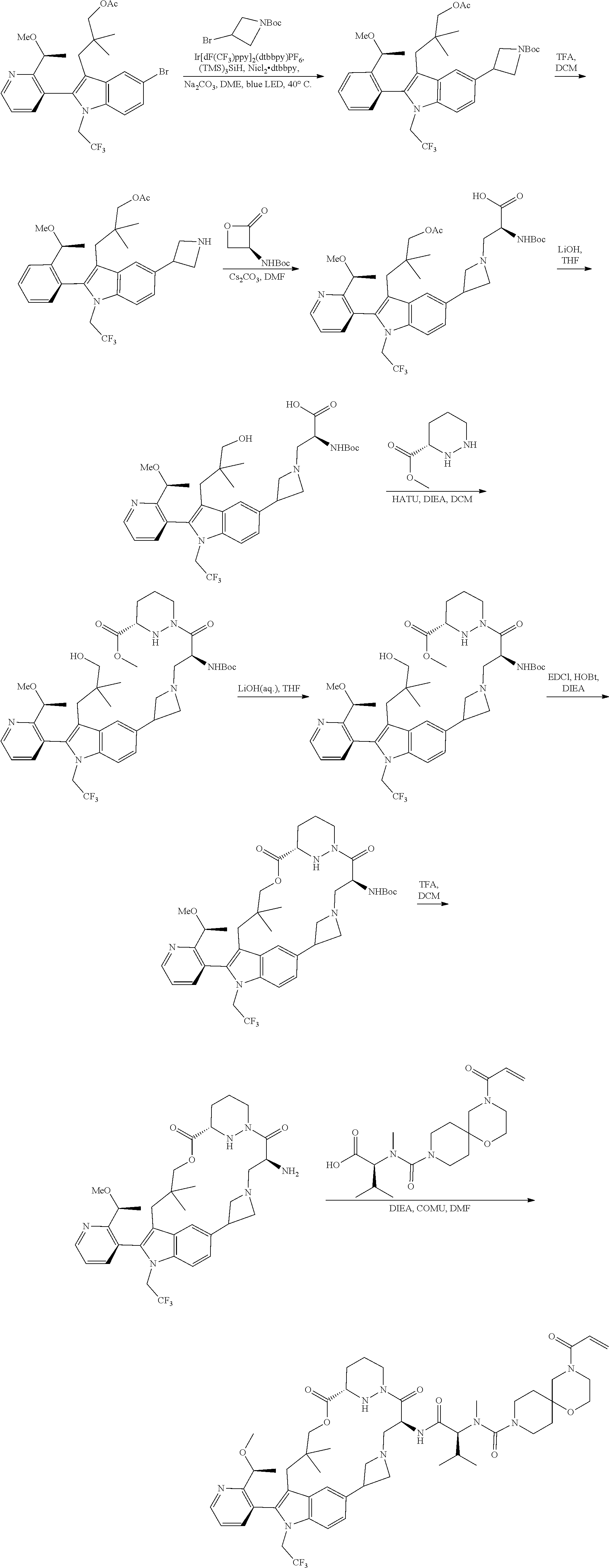

Step 1. To a 40 mL vial equipped with a stir bar was added photocatalyst Ir[dF(CF₃)ppy]₂(dtbbpy)PF6 (62 mg, 0.055 mmol), methyl 4-bromobenzoate (1.5 g, 2.8 mmol), 4-bromotetrahydropyran (981 mg, 4.2 mmol) tris(trimethylsilyl)silane (689 mg, 2.8 mmol), and anhydrous sodium carbonate (587 mg, 5.54 mmol). The vial was sealed and placed under an atmosphere of N₂ then DME (15 mL) added. To a separate vial was added NiCl₂⋅glyme (6.1 mg, 0.028 mmol) and 4,4′-di-tert-butyl-2,2′-bipyridine (7.4 mg, 0.028 mmol). The catalyst vial was sealed, purged with N₂ and DME (2 mL) was added, then this mixture was sonicated 5 min, after which, the mixture was added to the photocalatyst. The mixture was degassed with N₂ for 10 min, then the mixture was sealed and stirred under irradiation from a 34 W blue LED lamp (7 cm away, with a cooling fan to keep the reaction temperature at rt. The mixture was stirred at rt for 6 h, then H₂O was added and the mixture extracted with EtOAc (3×30 mL). The combined organic layers were washed with brine (30 mL), dried over anhydrous Na₂SO₄ and filtered. The filtrate was concentrated under reduced pressure and the crude residue was purified by silica gel column chromatography to give tert-butyl 3-[(2M)-3-[3-(acetyloxy)-2,2-dimethylpropyl]-2-{2-[(1S)-1-methoxyethyl]pyridin-3-yl}-1-(2,2,2-trifluoroethyl)indol-5-yl]azetidine-1-carboxylate (700 mg, 41% yield) as a solid. LCMS (ESI): m/z [M+H]⁺ calc'd for C₃₃H₄₂F₃N₃O₅ 617.3; found 618.4.

Step 2. To a mixture of tert-butyl 3-[(2M)-3-[3-(acetyloxy)-2,2-dimethylpropyl]-2-{2-[(1 S)-1-methoxyethyl]pyridin-3-yl}-1-(2,2,2-trifluoroethyl)indol-5-yl]azetidine-1-carboxylate (800 mg, 1.3 mmol) in DCM (8 mL) at 0° C. was added TFA (2.95 g, 25.9 mmol). The mixture was warmed to rt and stirred for 2 h, then concentrated under reduced pressure and the residue was basified to pH ˜8 with saturated NaHCO₃ and extracted with EtOAc (3×30 mL). The combined organic layers were washed with brine (30 mL), dried over anhydrous Na₂SO₄, filtered and the filtrate was concentrated under reduced pressure to give 3-[(2M)-5-(azetidin-3-yl)-2-{2-[(1S)-1-methoxyethyl]pyridin-3-yl}-1-(2,2,2-trifluoroethyl)indol-3-yl]-2,2-dimethylpropyl acetate (650 mg, 97%) as a solid. LCMS (ESI): m/z [M+H]⁺ calc'd for C₂₈H₃₄F₃N₃O₃ 517.3; found 518.3.

Step 3. To a mixture of 3-[(2M)-5-(azetidin-3-yl)-2-{2-[(1 S)-1-methoxyethyl]pyridin-3-yl}-1-(2,2,2-trifluoroethyl)indol-3-yl]-2,2-dimethylpropyl acetate (900 mg, 1.7 mmol) in DMF (9 mL) was added tert-butyl N-[(3S)-2-oxooxetan-3-yl]carbamate (488 mg, 2.6 mmol) and Cs₂CO₃ (567 mg, 1.7 mmol). The mixture was heated to 40° C. and stirred for 2 h, then H₂O was added and the mixture extracted with EtOAc (3×30 mL). The combined organic layers were washed with brine (30 mL), dried over anhydrous Na₂SO₄ and filtered. After filtration, the filtrate was concentrated under reduced pressure. The filtrate was concentrated under reduced pressure and the crude residue was purified by preparative-HPLC to give (2S)-3-{3-[(2M)-3-[3-(acetyloxy)-2,2-dimethylpropyl]-2-{2-[(1S)-1-methoxyethyl]pyridin-3-yl}-1-(2,2,2-trifluoroethyl)indol-5-yl]azetidin-1-yl}-2-[(tert-butoxycarbonyl)amino]propanoic acid (400 mg, 33% yield) as a solid. LCMS (ESI): m/z [M+H]⁺ calc'd for C₃₆H₄₇F₃N₄O₇ 704.3; found 705.4.

Step 4. To a mixture of (2S)-3-{3-[(2M)-3-[3-(acetyloxy)-2,2-dimethylpropyl]-2-{2-[(1 S)-1-methoxyethyl]pyridin-3-yl}-1-(2,2,2-trifluoroethyl)indol-5-yl]azetidin-1-yl}-2-[(tert-butoxycarbonyl)amino]propanoic acid (400 mg, 0.57 mmol) in THE (2.8 mL) at 0° C. was added 1M LiOH (2.84 mL, 2.84 mmol). The mixture was stirred at 0° C. for 2 h, then diluted with DCM (30 mL). The organic layer was washed with H₂O (3×30 mL) and the combined aqueous layers were acidified to pH ˜5 with 1M HCl, then extracted with EtOAc (3×40 mL). The combined organic layers were washed with brine (40 mL), dried over anhydrous Na₂SO₄, filtered and the filtrate was concentrated under reduced pressure to give (2S)-2-[(tert-butoxycarbonyl)amino]-3-{3-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]azetidin-1-yl}propanoic acid (300 mg, 80%) as a solid. LCMS (ESI): m/z [M+H]⁺ calc'd for C₃₄H₄₅F₃N₄O₆ 662.3; found 663.4.

Step 5. To a mixture of (2S)-2-[(tert-butoxycarbonyl)amino]-3-{3-[(2M)-3-(3-hydroxy-2,2-dimethylpropyl)-2-{2-[(1 S)-1-methoxyethyl]pyridin-3-yl}-1-(2,2,2-trifluoroethyl)indol-5-yl]azetidin-1-yl}propanoic acid (300 mg, 0.45 mmol) in DCM (3 mL) at 0° C. was added DIPEA (351 mg, 2.7 mmol), methyl (3S)-1,2-diazinane-3-carboxylate (131 mg, 0.91 mmol) and HATU (258 mg, 0.68 mmol). The mixture was stirred at 0° C. for 3 h, then H₂O was added and the mixture extracted with DCM (3×30 mL). The combined organic layers were dried over anhydrous Na₂SO₄, filtered, the filtrate was concentrated under reduced pressure and the crude residue was purified by preparative-TLC to give methyl (3S)-1-[(2S)-2-[(tert-butoxycarbonyl)amino]-3-{3-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]azetidin-1-yl}propanoyl]-1,2-diazinane-3-carboxylate (290 mg, 81%) as a solid. LCMS (ESI): m/z [M+H]⁺ calc'd for C₄₀H₅₅F₃N₆O₇ 788.4; found 789.5.

Step 6. To a mixture of methyl (3S)-1-[(2S)-2-[(tert-butoxycarbonyl)amino]-3-{3-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]azetidin-1-yl}propanoyl]-1,2-diazinane-3-carboxylate (290 mg, 0.37 mmol) in THE (1.8 mL) at 0° C. was added 1M LiOH (1.84 mL, 1.84 mmol). The mixture was stirred at 0° C. for 1 h, then DCM (20 mL) was added and the mixture washed with H₂O (3×30 mL). The combined aqueous layers were acidified to pH ˜5 with 1M HCl and the mixture was extracted with EtOAc (3×60 mL). The combined organic layers were dried over anhydrous Na₂SO₄, filtered and the filtrate was concentrated under reduced pressure to give (3S)-1-[(2S)-2-[(tert-butoxycarbonyl)amino]-3-{3-[(2M)-3-(3-hydroxy-2,2-dimethylpropyl)-2-{2-[(1S)-1-methoxyethyl]pyridin-3-yl}-1-(2,2,2-trifluoroethyl)indol-5-yl]azetidin-1-yl}propanoyl]-1,2-diazinane-3-carboxylic acid (230 mg, 81% yield) as a solid. LCMS (ESI): m/z [M+H]⁺ calc'd for C₃₉H₅₃F₃N₆O₇ 774.4; found 775.5.

Step 7. To a mixture of (3S)-1-[(2S)-2-[(tert-butoxycarbonyl)amino]-3-{3-[(2M)-3-(3-hydroxy-2,2-dimethylpropyl)-2-{2-[(1 S)-1-methoxyethyl]pyridin-3-yl}-1-(2,2,2-trifluoroethyl)indol-5-yl]azetidin-1-yl}propanoyl]-1,2-diazinane-3-carboxylic acid (280 mg, 0.36 mmol) in DCM (56 mL) was added DIPEA (1.4 g, 10.8 mmol), HOBT (293 mg, 2.2 mmol) and EDCI (2.1 g, 10.8 mmol). The mixture was warmed to 30° C. and stirred overnight the H₂O was added and the mixture extracted with DCM (3×50 mL). The combined organic layers were dried over anhydrous Na₂SO₄, filtered, the filtrate was concentrated under reduced pressure and the residue was purified by preparative-TLC to give tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-azetidinacycloundecaphane-4-yl)carbamate (100 mg, 37% yield) as a solid. LCMS (ESI): m/z [M+H]⁺ calc'd for C₃₉H₅₁F₃N₆O₆ 756.4; found 757.4.

Step 8. To a mixture of tert-butyl ((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-azetidinacycloundecaphane-4-yl)carbamate (100 mg, 0.13 mmol) in DCM (2 mL) at 0° C. was added TFA (301 mg, 2.64 mmol). The mixture was stirred at 0° C. for 4 h, then concentrated under reduced pressure to give (6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)- azetidinacycloundecaphane-5,7-dione (80 mg, 92% yield) as a solid. LCMS (ESI): m/z [M+H]⁺ calc'd for C₃₄H₄₃F₃N₆O₄ 656.3; found 657.5.

Step 9. To a mixture of (6³S,4S)-4-amino-1²-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)- azetidinacycloundecaphane-5,7-dione (90 mg, 0.14 mmol) in DMF (2 mL) at 0° C. was added DIPEA (106 mg, 0.82 mmol), (2S)-3-methyl-2-[methyl(4-(prop-2-enoyl)-1-oxa-4,9-diazaspiro[5.5]undecane-9-carbonyl)amino]butanoic acid (76 mg, 0.21 mmol) and COMU (88 mg, 0.21 mmol). The mixture was stirred at 0° C. for 1 h, then H₂O was added and the mixture was extracted with EtOAc (3×20 mL). The combined organic layers were washed with brine (20 mL), dried over anhydrous Na₂SO₄ and filtered. The filtrate was concentrated under reduced pressure and the residue was purified by preparative-HPLC to give 4-acryloyl-N-((2S)-1-(((6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)- azetidinacycloundecaphane-4-yl)amino)-3-methyl-1-oxobutan-2-yl)-N-methyl-1-oxa-4,9-diazaspiro[5.5]undecane-9-carboxamide (37 mg, 27% yield) as a solid. LCMS (ESI): m/z [M+H]⁺ calc'd for C₅₂H₇₀F₃N₉O₈ 1005.5; found 1006.8; ¹H NMR (400 MHz, DMSO-d₆) δ 8.73 (dd, J=4.7, 1.8 Hz, 1H), 7.80 (s, 1H), 7.71-7.69 (m, 2H), 7.58-7.46 (m, 2H), 7.10 (d, J=8.4 Hz, 1H), 6.86-6.71 (m, 1H), 6.11 (dd, J=16.3, 9.7 Hz, 1H), 5.65 (t, J=8.3 Hz, 1H), 5.46 (dq, J=17.2, 8.8 Hz, 1H), 5.29-5.15 (m, 2H), 4.87-4.74 (m, 1H), 4.23 (d, J=12.3 Hz, 1H), 4.11 (q, J=6.0 Hz, 1H), 4.07-3.97 (m, 1H), 3.86-3.71 (m, 2H), 3.61-3.47 (m, 12H), 3.23 (m, 5H), 3.07-2.87 (m, 5H), 2.78 (s, 3H), 2.76-2.66 (m, 1H), 2.32 (d, J=14.4 Hz, 1H), 2.18-2.05 (m, 1H), 2.04-1.94 (m, 1H), 1.78 (d, J=10.0 Hz, 1H), 1.71 (d, J=13.3 Hz, 1H), 1.58 (dd, J=16.6, 6.9 Hz, 4H), 1.48-1.38 (m, 1H), 1.32 (d, J=6.0 Hz, 3H), 0.88-0.75 (m, 9H), 0.24 (s, 3H).

### Synthesis of (2R)-3-acryloyl-N-((2S)-1-(((6³S,4S)-11-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina- 2(5,1)-pyridinacycloundecaphane-4-yl)amino)-3-methyl-1-oxobutan-2-yl)-N,2-dimethyl-1-oxa-3,8-diazaspiro[4.5]decane-8-carboxamide

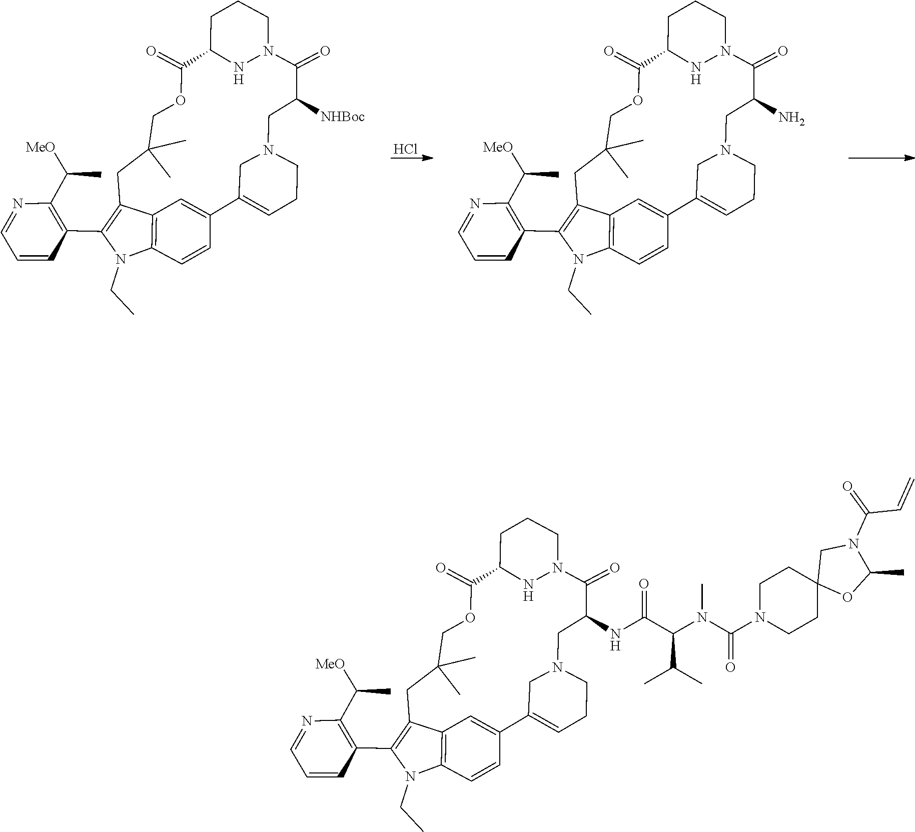

Step 1. To a mixture of methyl N—((R)-3-acryloyl-2-methyl-1-oxa-3,8-diazaspiro[4.5]decane-8-carbonyl)-N-methyl-L-valinate (430 mg, 1.127 mmol, 1.00 equiv) in THE (4 mL) and H₂O (4 mL) was added NaOH (225 mg, 5.6 mmol). The mixture was stirred at rt for 16 hours at rt, then acidified to pH ˜5 with 1M HCl and the mixture was extracted with EtOAc (4×10 mL). The combined organic layers were washed with brine (3 mL), dried over anhydrous Na₂SO₄, filtered and the filtrate was concentrated under reduced pressure to give N—((R)-3-acryloyl-2-methyl-1-oxa-3,8-diazaspiro[4.5]decane-8-carbonyl)-N-methyl-L-valine (300 mg) as a solid. LCMS (ESI): m/z [M+H]⁺ calc'd for C₁₈H₂₉N₃O₅ 367.2; found 368.3.

Step 2. To a mixture of tert-butyl ((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6(1,3)-pyridazina-2(5,1)-pyridinacycloundecaphane-4-yl)carbamate (1.0 g, 1.4 mmol) in DCM (10 mL) at 0° C. was added HCl in 1,4-dioxane (5 mL). The mixture was stirred at 0° C. for 2 h, then concentrated under reduced pressure to give (6³S,4S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-5,7-dione HCl (1.0 g) as a solid. LCMS (ESI): m/z [M+H]⁺ calc'd for C₃₆H₄₈N₆O₄ 628.4; found 629.6.

Step 3. To a mixture of (6³S,4S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1 (5,3)-indola-6(1,3)-pyridazina-2(5,1)- pyridinacycloundecaphane-5,7-dione HCl (460 mg, 0.73 mmol) and N—((R)-3-acryloyl-2-methyl-1-oxa-3,8-diazaspiro[4.5]decane-8-carbonyl)-N-methyl-L-valine (269 mg, 0.73 mmol) in DMF (5 mL) at 0° C. was added DIPEA (2.84 g, 22.0 mmol) and COMU (282 mg, 0.66 mmol). The mixture was stirred at 0° C. for 1 h, then H₂O was added and the mixture extracted with EtOAc (5×10 mL). The combined organic layers were washed with brine (3×6 mL), dried over anhydrous Na₂SO₄ and filtered. The filtrate was concentrated under reduced pressure and the residue was purified by preparative-HPLC to give (2R)-3-acryloyl-N-((2S)-1-(((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6(1,3)-pyridazina-2(5,1)- pyridinacycloundecaphane-4-yl)amino)-3-methyl-1-oxobutan-2-yl)-N,2-dimethyl-1-oxa-3,8-diazaspiro[4.5]decane-8-carboxamide (50 mg, 7% yield) as a solid. LCMS (ESI): m/z [M+H]⁺ calc'd for C₅₄H₇₅N₉O₈ 977.6; found 978.6; ¹H NMR (400 MHz, DMSO-d₆) δ 8.83-8.67 (m, 1H), 7.89 (dd, J=18.7, 8.2 Hz, 2H), 7.62-7.33 (m, 4H), 6.57 (dd, J=16.7, 10.3 Hz, 1H), 6.38-6.11 (m, 2H), 5.75 (d, J=9.8 Hz, 2H), 5.61 (d, J=11.8 Hz, 1H), 5.35 (d, J=5.5 Hz, 1H), 4.30 (d, J=12.7 Hz, 1H), 4.16 (q, J=6.2 Hz, 1H), 4.04 (s, 2H), 3.92-3.68 (m, 4H), 3.63 (s, 2H), 3.18 (d, J=61.5 Hz, 6H), 2.95 (d, J=33.8 Hz, 5H), 2.78 (t, J=11.8 Hz, 1H), 2.64 (d, J=24.7 Hz, 7H), 2.42-1.83 (m, 7H), 1.89-1.45 (m, 7H), 1.40 (dd, J=11.9, 5.7 Hz, 6H), 1.10 (t, J=7.0 Hz, 3H), 0.94-0.64 (m, 9H), 0.52 (s, 3H).

### Synthesis of 3-acryloyl-N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-pyrrolidinacycloundecaphane-4-yl)amino)-3-methyl-1-oxobutan-2-yl)-N-methyl-1-oxa-3,8-diazaspiro[4.5]decane-8-carboxamide

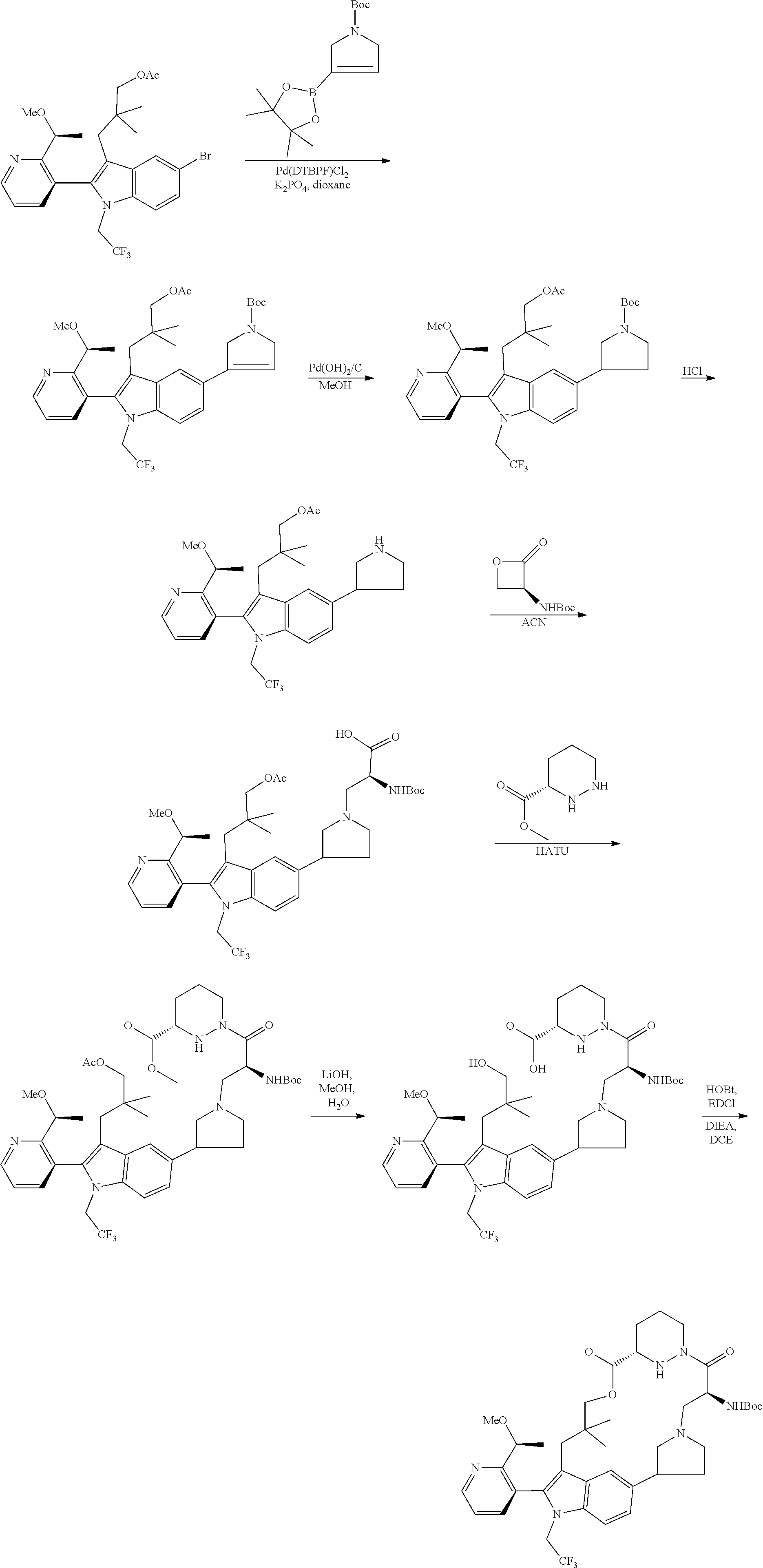

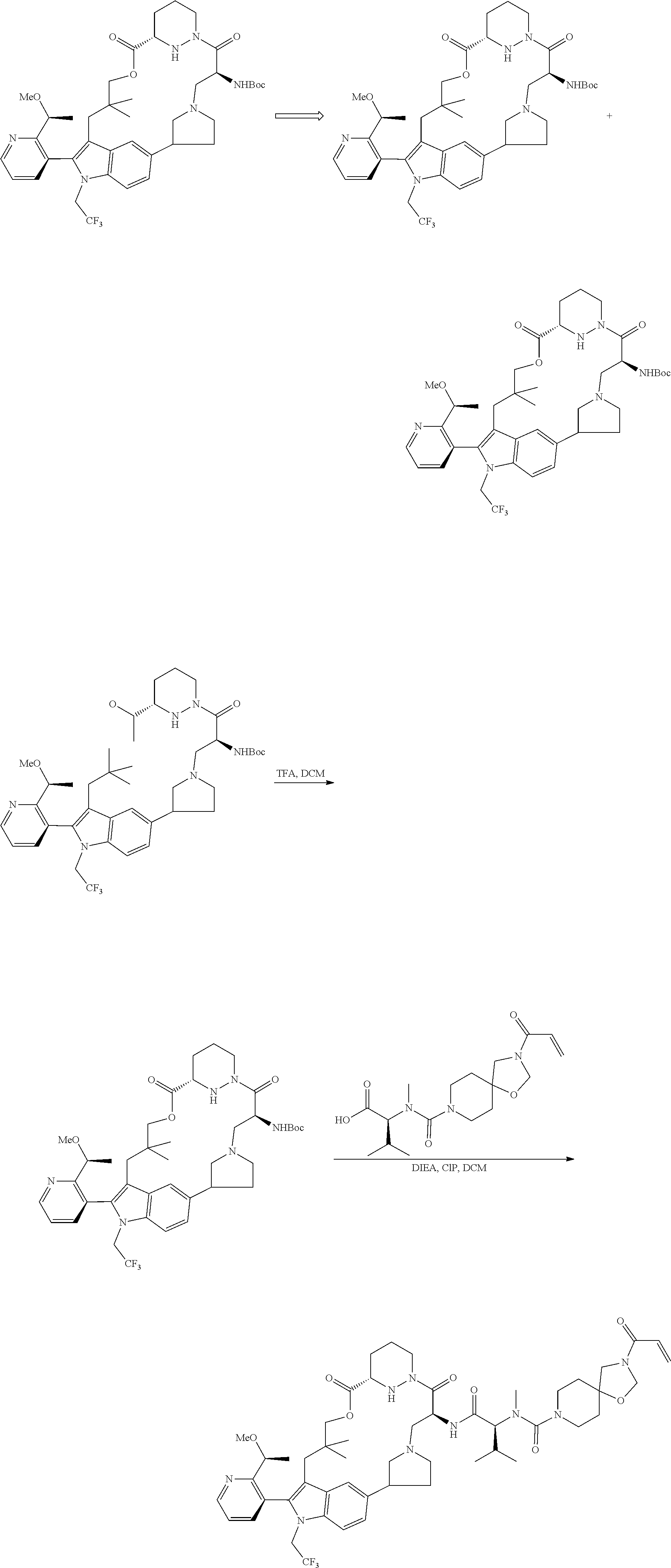

Step 1. To a mixture of (S)-3-(5-bromo-2-(2-(1-methoxyethyl) pyridin-3-yl)-1-(2,22-trifluoroethyl)-1H-indol-3-yl)-2,2-dimethylpropyl acetate (10 g, 18.5 mmol) and tert-butyl 3-(4,4,5,5-tetramethyl-1,3,2-PP-3723, C₃ dioxaborolan-2-yl)-2,5-dihydro-1H-pyrrole-1-carboxylate (8.18 g, 27.7 mmol) in dioxane (100 mL) and H₂O (20 mL) under an atmosphere of Ar was added Pd(DTBPF)C₁₂ (1.20 g, 1.85 mmol) and K₃PO₄ (9.80 g, 46.2 mmol). The mixture was heated to 85° C. and stirred for 1 h, then extracted with EtOAc (10 m. The combined organic layers were washed with brine (8×5 mL, dried over anhydrous Na₂SO₄ and filtered. The filtrate was concentrated under reduced pressure and the residue was purified by silica gel column chromatography to give tert-butyl (S)-3-(3-(3-acetoxy-2,2-dimethyl propyl)-2-(2-(1-methoxyethyl) pyridin-3-yl)-1-(2,2,2-trifluoroethyl)-1H-indol-5-yl)-2,5-dihydro-1H-pyrrole-1-carboxylate (13 g, 89% yield) as an oil. LCMS (ESI): m/z [M+H]⁺ calc'd for C₃₄H₄₂F₃N₃O₅ 629.3; found 630.4.

Step 2. A mixture of tert-butyl (S)-3-(3-(3-acetoxy-2,2-dimethyl propyl)-2-(2-(1-methoxyethyl) pyridin-3-yl)-1-(2,2,2-trifluoroethyl)-1H-indol-5-yl)-2,5-dihydro-1H-pyrrole-1-carboxylate (10.75 g, 17.1 mmol) and Pd(OH)₂/C (3.2 g, 22.8 mmol) in MeOH (100 mL) was heated to 40° C. and under an atmosphere of H₂ for 2 h. The mixture was filtered and the filter cake was washed with DCM (10×10 mL). The filtrate was concentrated under reduced pressure and the residue was purified by preparative-HPLC to give tert-butyl 3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidine-1-carboxylate (6.4 g, 56% yield) as an oil. LCMS (ESI): m/z [M+H]⁺ calc'd for C₃₄H₄₄F₃N₃O₅ 631.3; found 632.4.

Step 3. To a mixture of tert-butyl 3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidine-1-carboxylate (7.0 g, 11.1 mmol) in dioxane (70 mL) was added HCl in 1,4-dioxane (17.5 mL). The mixture was stirred at rt for 1 h, then concentrated under reduced pressure to give 3-(2-(2-((S)-1-methoxyethyl)pyridin-3-yl)-5-(pyrrolidin-3-yl)-1-(2,2,2-trifluoroethyl)-1H-indol-3-yl)-2,2-dimethylpropyl acetate (7.6 g) as an oil. LCMS (ESI): m/z [M+H]⁺ calc'd for C₂₉H₃₆F₃N₃O₃ 531.3; found 532.5.

Step 4. To a mixture of 3-(2-(2-((S)-1-methoxyethyl)pyridin-3-yl)-5-(pyrrolidin-3-yl)-1-(2,2,2-trifluoroethyl)-1H-indol-3-yl)-2,2-dimethylpropyl acetate (7.7 g, 14.5 mmol) in ACN (80 mL) was added tert-butyl (S)-(2-oxooxetan-3-yl)carbamate (4.07 g, 21.7 mmol) and Cs₂CO₃ (11.80 g, 36.2 mmol). The mixture was heated to 40° C. and stirred for 2 h, then acidified to pH ˜7 with conc. HCl and the mixture was extracted with EtOAc (500 mL). The combined organic layers were washed with brine (3×100 mL), dried over anhydrous Na₂SO₄ and filtered. The filtrate was concentrated under reduced pressure and the residue was purified by preparative-HPLC to give (2S)-3-(3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)-2-((tert-butoxycarbonyl)amino)propanoic acid (2.3 g, 19% yield) as an oil. LCMS (ESI): m/z [M+H]⁺ calc'd for C₃₇H₄₉F₃N₄O₇ 718.4; found 719.5.

Step 5. To a mixture of methyl (S)-hexahydropyridazine-3-carboxylate (0.69 g, 4.8 mmol), DIPEA (16.54 g, 128 mmol) and (2S)-3-(3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)-2-((tert-butoxycarbonyl)amino)propanoic acid (2.3 g, 3.2 mmol) in DCM (60 mL) at 0° C. under an atmosphere of N₂ was added HATU (1.46 g, 3.84 mmol) in portions. The resulting mixture was warmed to rt and stirred for 1 h, the H₂O was added and the mixture extracted with EtOAc (200 mL). The combined organic layers were washed with brine (3×400 mL), dried over anhydrous Na₂SO₄ and filtered. The filtrate was concentrated under reduced pressure and the residue was purified by preparative-HPLC to give methyl (3S)-1-((2S)-3-(3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)-2-((tert-butoxycarbonyl)amino)propanoyl)hexahydropyridazine-3-carboxylate (2 g, 70% yield) as an oil.

LCMS (ESI): m/z [M+H]⁺ calc'd for C₄₃H₅₉F₃N₆O₈ 844.4; found 845.6.

Step 6. A mixture of methyl (3S)-1-((2S)-3-(3-(3-(3-acetoxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)-2-((tert-butoxycarbonyl)amino)propanoyl)hexahydropyridazine-3-carboxylate (2.0 g, 2.4 mmol) and LiOH (0.28 g, 11.8 mmol) in H₂O (10 mL) and MeOH (20 mL) was stirred at rt. The mixture was acidified to pH ˜6 with aqueous HCl and the mixture extracted with DCM (4× mL). The combined organic layers were washed with brine (6×4 mL), dried over anhydrous Na₂SO₄, filtered and the filtrate was concentrated under reduced pressure to give (3S)-1-((2S)-2-((tert-butoxycarbonyl)amino)-3-(3-(3-(3-hydroxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)propanoyl)hexahydropyridazine-3-carboxylic acid (1.9 g) as an oil. LCMS (ESI): m/z [M+H]⁺ calc'd for C₄₀H₅₅F₃N₆O₇ 788.4; found 789.4.

Step 7. To a mixture of (3S)-1-((2S)-2-((tert-butoxycarbonyl)amino)-3-(3-(3-(3-hydroxy-2,2-dimethylpropyl)-2-(2-((S)-1-methoxyethyl)pyridin-3-yl)-1-(2,2,2-trifluoroethyl)-1H-indol-5-yl)pyrrolidin-1-yl)propanoyl)hexahydropyridazine-3-carboxylic acid (1.87 g, 2.4 mmol) in DCM (340 mL) under an atmosphere of N₂ was added DIPEA (9.19 g, 71.1 mmol), HOBt (1.60 g, 11.9 mmol) and EDCI (9.09 g, 47.4 mmol). The mixture was stirred at rt overnight, then H₂O was added and the mixture extracted with DCM (2× mL). The combined organic layers were washed with brine (3×3 mL) dried over anhydrous Na₂SO₄ and filtered. The filtrate was concentrated under reduced pressure and the residue was purified by silica gel column chromatography to give tert-butyl ((6³S,4S)-12-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-pyrrolidinacycloundecaphane-4-yl)carbamate (410 mg, 21% yield) as a solid.

Step 8. Diastereomers were separated by use of silica gel column chromatography to give each respective isomer.

Data for Isomer 1 (R_{f}=0.4 in 1:1 petroleum ether/EtOAc): LCMS (ESI): m/z [M+H]⁺ calc'd for C₄₀H₅₃F₃N₆O₆ 770.4; found 771.4.

Data for Isomer 2 (R_{f}=0.7 in 1:1 petroleum ether/EtOAc): LCMS (ESI): m/z [M+H]⁺ calc'd for C₄₀H₅₃F₃N₆O₆ 770.4; found 771.4.

Step 9. To a mixture of tert-butyl ((6³S,4S)-12-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-pyrrolidinacycloundecaphane-4-yl)carbamate (410 mg, 0.53 mmol) in DCM (5 mL) at 0° C. was added TFA (1.7 mL, 22.9 mmol). The mixture was warmed to rt and stirred for 1 h, then basified to pH ˜6 with saturated NaHCO₃ and the mixture was extracted with EtOAc (6×3 mL). The combined organic layers were washed with brine (5×3 mL), dried over anhydrous Na₂SO₄, filtered and the filtrate was concentrated under reduced pressure to give (2³S,6³S,4S)-4-amino-12-(2-((S)-1-methoxyethyl) pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-pyrrolidinacycloundecaphane-5,7-dione (390 mg) as a solid. LCMS (ESI): m/z [M+H]⁺ calc'd for C₃₅H₄₅F₃N₆O₄ 670.4; found 671.7.

Step 10. To a mixture of (2³S,6³S,4S)-4-amino-12-(2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(3,1)-pyrrolidinacycloundecaphane-5,7-dione (270 mg, 0.4 mmol) and DIPEA (2.1 g, 16.1 mmol) in DCM (3 mL) at 0° C. under an atmosphere of N₂ was added (2S)-3-methyl-2-[methyl(3-(prop-2-enoyl)-1-oxa-3,8-diazaspiro[4.5]decane-8-carbonyl)amino]butanoic acid (142 mg, 0.4 mmol) and CIP (227 mg, 0.81 mmol). The mixture was stirred at rt for 30 min, then H₂O was added and the mixture extracted with EtOAc (4×30 mL). The combined organic layers were washed with brine (5×30 mL), dried over anhydrous Na₂SO₄ and filtered. The filtrate was concentrated under reduced pressure and the residue was purified by preparative-HPLC to give 3-acryloyl-N-((2S)-1-(((2³S,6³S,4S)-1²-(2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1 (5,3)-indola-6(1,3)-pyridazina-2(3,1)-pyrrolidinacycloundecaphane-4-yl)amino)-3-methyl-1-oxobutan-2-yl)-N-methyl-1-oxa-3,8-diazaspiro[4.5]decane-8-carboxamide (45 mg, 10% yield) as a solid. LCMS (ESI): m/z [M+H]⁺ calc'd for C₅₂H₇₀F₃N₉O₈ 1005.5; found 1006.9; ¹H NMR (400 MHz, DMSO-d₆) δ 8.76 (dd, J=4.7, 1.8 Hz, 1H), 7.81 (d, J=8.8 Hz, 1H), 7.74 (d, J=7.7 Hz, 1H), 7.60 (d, J=8.4 Hz, 1H), 7.58-7.50 (m, 2H), 7.13 (d, J=8.2 Hz, 1H), 6.54 (dd, J=16.8, 10.3 Hz, 1H), 6.24-6.14 (m, 1H), 5.74 (td, J=10.2, 2.3 Hz, 1H), 5.58 (q, J=6.9 Hz, 1H), 5.46 (dt, J=17.2, 8.7 Hz, 1H), 5.13 (d, J=13.2 Hz, 2H), 5.01 (s, 1H), 4.81 (dt, J=18.2, 9.0 Hz, 1H), 4.31 (d, J=12.4 Hz, 1H), 4.20 (q, J=6.0 Hz, 1H), 3.87 (s, 1H), 3.80 (d, J=11.0 Hz, 1H), 3.67 (s, 2H), 3.60-3.55 (m, 1H), 3.45 (s, 1H), 3.12 (dt, J=17.2, 9.6 Hz, 3H), 2.76 (d, J=13.0 Hz, 5H), 2.61 (q, J=7.8, 6.9 Hz, 2H), 2.42 (d, J=14.4 Hz, 1H), 2.29-1.87 (m, 4H), 1.80 (t, J=12.5 Hz, 3H), 1.65 (dt, J=22.2, 8.9 Hz, 3H), 1.58-1.48 (m, 2H), 1.38 (d, J=6.0 Hz, 3H), 0.98-0.83 (m, 6H), 0.81 (d, J=6.6 Hz, 3H), 0.26 (s, 3H).

### Synthesis of 4-acryloyl-N-((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-1-oxa-4,9-diazaspiro[5.5]undecane-9-carboxamide

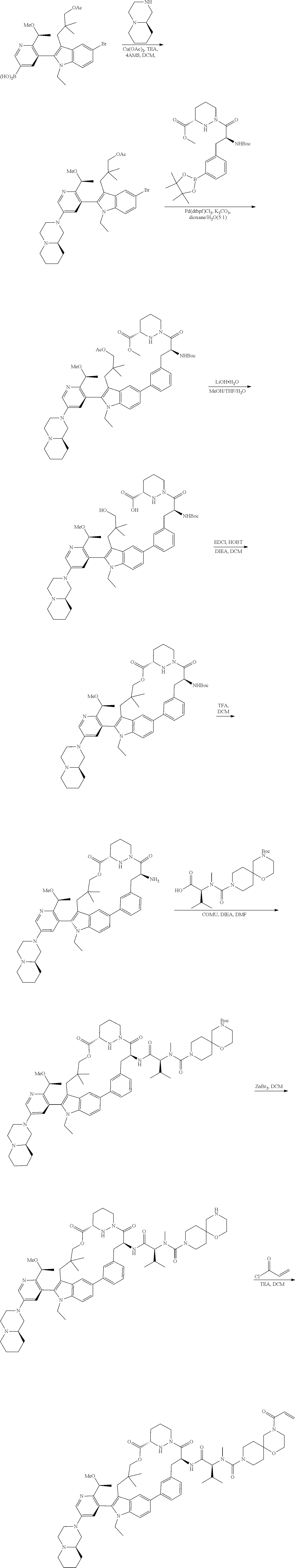

Step 1. To a mixture of (S)-(5-(3-(3-acetoxy-2,2-dimethylpropyl)-5-bromo-1-ethyl-1H-indol-2-yl)-6-(1-methoxyethyl)pyridin-3-yl)boronic acid (7.7 g, 14.5 mmol) and (R)-octahydro-2H-pyrido[1,2-a]pyrazine (3.9 g, 27.8 mmol) in DCM (230 mL) under an atmosphere of 02 was added TEA (14.7 g, 145.3 mmol) and 4 Å molecular sieves (26 g). The mixture was stirred at rt for 30 min, then Cu(OAc)₂ (2.4 g, 13.2 mmol) was added, the mixture heated to 40° C. and stirred overnight. Ice/H₂O was added and the mixture was extracted with EtOAc (5×200 mL). The combined organic layers were washed with brine, dried over anhydrous Na₂SO₄ and filtered. The filtrate was concentrated under reduced pressure and the residue was purified to give 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (3.5 g, 27% yield) as a solid. LCMS (ESI): m/z [M+H]⁺ calc'd for C₃₃H₄₅BrN₄O₃ 624.3; found 625.5.

Step 2. To a mixture of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (1.9 g, 3.0 mmol) and methyl (S)-1-((S)-2-((tert-butoxycarbonyl)amino)-3-(3-(4,4,5,5-tetramethyl-1,3,2-dioxaborolan-2-yl)phenyl)propanoyl)hexahydropyridazine-3-carboxylate (1.89 g, 3.6 mmol) in dioxane (19 mL) and H₂O (3.8 mL) was added K₂CO₃ (1.05 g, 7.6 mmol) and Pd(dtbpf)Cl₂ (395 mg, 0.61 mmol). The mixture was heated to 70° C. and stirred for 3 h, then diluted with EtOAc (40 mL), ice/H₂O added, and the mixture was extracted with EtOAc (3×10 mL). The combined organic layers were washed with brine, dried over anhydrous Na₂SO₄ and filtered. The filtrate was concentrated under reduced pressure and the residue was purified to give methyl (S)-1-((S)-3-(3-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)phenyl)-2-((tert-butoxycarbonyl)amino)propanoyl)hexahydropyridazine-3-carboxylate (1.1 g, 29% yield) as a solid. LCMS (ESI): m/z [M+H]⁺ calc'd for C₅₃H₇₃N₇O₈ 935.6; found 936.8.

Step 3. To a mixture of methyl (S)-1-((S)-3-(3-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)phenyl)-2-((tert-butoxycarbonyl)amino)propanoyl)hexahydropyridazine-3-carboxylate (900 mg, 0.92 mmol) in THE (4.5 mL), MeOH (4.5 mL) and H₂O (4.5 mL) at 0° C. was added LiOH·H₂O (89 mg, 3.7 mmol). The mixture was warmed to rt and stirred for 3 h, then ice/H₂O (10 mL) added, the mixture acidified to pH ˜5 with citric acid and the mixture was extracted with EtOAc (3×20 mL). The combined organic layers were washed with brine, dried over anhydrous Na₂SO₄, filtered and the filtrate was concentrated under reduced pressure to give (S)-1-((S)-2-((tert-butoxycarbonyl)amino)-3-(3-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)phenyl)propanoyl)hexahydropyridazine-3-carboxylic acid (900 mg) as a solid. LCMS (ESI): m/z [M+H]⁺ calc'd for C₅₀H₆₉N₇O₇ 879.5; found 880.6.

Step 4. To a mixture of (S)-1-((S)-2-((tert-butoxycarbonyl)amino)-3-(3-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)phenyl)propanoyl)hexahydropyridazine-3-carboxylic acid (670 mg, 0.76 mmol) in DCM (67 mL) at 0° C. was added DIPEA (3.94 g, 30.4 mmol), EDCI (4.4 g, 22.8 mmol) and HOBT (514 mg, 3.8 mmol). The mixture was warmed to rt and stirred overnight, then ice/H₂O (100 mL) was added and the mixture extracted with EtOAc (3×100 mL). The combined organic layers were washed with saturated NH₄Cl (3×100 mL), dried over anhydrous Na₂SO₄ and filtered. The filtrate was concentrated under reduced pressure and the residue was purified to give tert-butyl ((6³S,4S)-1′-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (450 mg, 62% yield) as a solid. LCMS (ESI): m/z [M+H]⁺ calc'd for C₅₀H₆₇N₇O₆ 861.5; found 862.7.

Step 5. To a mixture of tert-butyl ((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (230 mg, 0.27 mmol) in DCM (2 mL) at 0° C. was added TFA (1 mL) dropwise. The mixture was stirred at 0° C. for 1 h, then basified to pH ˜8 with saturated NaHCO₃ at 0° C. and the mixture extracted with EtOAc (3×30 mL). The combined organic layers were washed with brine (3×30 mL), dried over anhydrous Na₂SO₄, filtered and the filtrate was concentrated under reduced pressure to give (6³S,4S)-4-amino-11-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (300 mg) as a solid, that was used in the next step without further purification. LCMS (ESI): m/z [M+H]⁺ calc'd for C₄₅H₅₉N₇O₄ 761.5; found 762.8.

Step 6. To a mixture of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (300 mg, 0.39 mmol) and (2S)-2-[4-(tert-butoxycarbonyl)-1-oxa-4,9-diazaspiro[5.5]undecane-9-carbonyl(methyl)amino]-3-methylbutanoic acid (211 mg, 0.51 mmol) in DMF (3 mL) at 0° C. under an atmosphere of Ar was added DIPEA (1.53 g, 11.8 mmol) and COMU (168 mg, 0.39 mmol) in DMF (0.1 mL) dropwise. The mixture was stirred at 0° C. for 1 h, then ice/H₂O (3 mL) was added and the mixture extracted with EtOAc (3×30 mL). The combined organic layers were washed with brine (3×30 mL), dried over anhydrous Na₂SO₄ and filtered. The filtrate was concentrated under reduced pressure and the residue was purified to give tert-butyl 9-(((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-3-methyl-1-oxobutan-2-yl)(methyl)carbamoyl)-1-oxa-4,9-diazaspiro[5.5]undecane-4-carboxylate (200 mg, 59% yield) as a solid. LCMS (ESI): m/z [M+H]⁺ calc'd for C₆₅H₉₂N₁₀O₉ 1156.7; found 1158.2.

Step 7. A mixture of tert-butyl 9-(((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-1-oxa-4,9-diazaspiro[5.5]undecane-4-carboxylate (200 mg, 0.17 mmol) and ZnBr₂ (195 mg, 0.87 mmol) in DCM (4 mL) was heated to 35° C. and stirred overnight. Ice/H₂O (5 mL) was added and the mixture was basified to pH ˜8 with saturated NaHCO₃ at 0° C., then extracted with EtOAc (3×30 mL). The combined organic layers were washed with brine (3×30 mL), dried over anhydrous Na₂SO₄, filtered and the filtrate was concentrated under reduced pressure to give N-((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-3-methyl-1-oxobutan-2-yl)-N-methyl-1-oxa-4,9-diazaspiro[5.5]undecane-9-carboxamide (200 mg) as a solid. LCMS (ESI): m/z [M+H]⁺ calc'd for C₆₀H₈₄N₁₀O₇ 1056.7; found 1058.1.

Step 8. To a mixture of N-((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-1-oxa-4,9-diazaspiro[5.5]undecane-9-carboxamide (200 mg, 0.19 mmol) and TEA (57 mg, 0.57 mmol) in DCM (2 mL) at 0° C. under an atmosphere of Ar was added acryloyl chloride (12 mg, 0.13 mmol) dropwise. The mixture was stirred at 0° C. for additional 1 h, then concentrated under reduced pressure and the crude residue was purified by preparative-HPLC to give 4-acryloyl-N-((2S)-1-(((6³S,4S)-1′-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methyl-1-oxa-4,9-diazaspiro[5.5]undecane-9-carboxamide (40 mg, 19% yield) as a solid. LCMS (ESI): m/z [M+H]⁺ calc'd for C₆₃H₈₆N₁₀O₈ 1110.7; found 1112.1; ¹H NMR (400 MHz, DMSO-d₆) δ 8.46 (d, J=2.8 Hz, 1H), 8.17-8.05 (m, 1H), 7.98 (s, 1H), 7.86 (s, 1H), 7.74-7.54 (m, 3H), 7.27-7.19 (m, 2H), 7.01-6.81 (m, 2H), 6.28-6.11 (m, 1H), 5.73 (d, J=10.3 Hz, 1H), 5.43 (d, J=9.4 Hz, 2H), 4.40-4.17 (m, 2H), 4.10 (dq, J=21.9, 7.1, 6.5 Hz, 2H), 3.95 (t, J=12.0 Hz, 1H), 3.77 (dt, J=25.3, 13.0 Hz, 3H), 3.69-3.64 (m, 3H), 3.64-3.55 (m, 3H), 3.54-3.48 (m, 2H), 3.15 (d, J=11.7 Hz, 2H), 3.07 (s, 3H), 2.97-2.89 (m, 1H), 2.79 (m, 4H), 2.66 (s, 1H), 2.56 (s, 3H), 2.42 (d, J=11.1 Hz, 1H), 2.23 (td, J=11.6, 3.2 Hz, 1H), 2.07-1.89 (m, 4H), 1.82 (d, J=12.2 Hz, 1H), 1.77-1.63 (m, 4H), 1.59 (d, J=12.6 Hz, 3H), 1.47 (d, J=13.1 Hz, 2H), 1.36 (d, J=6.1 Hz, 3H), 1.19 (m, 3H), 1.00 (t, J=7.1 Hz, 3H), 0.90-0.70 (m, 9H), 0.57 (s, 3H).

### Synthesis of (3S)-1-acryloyl-N-((2S)-1-(((6³S)-11-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)-N-methylpyrrolidine-3-carboxamide

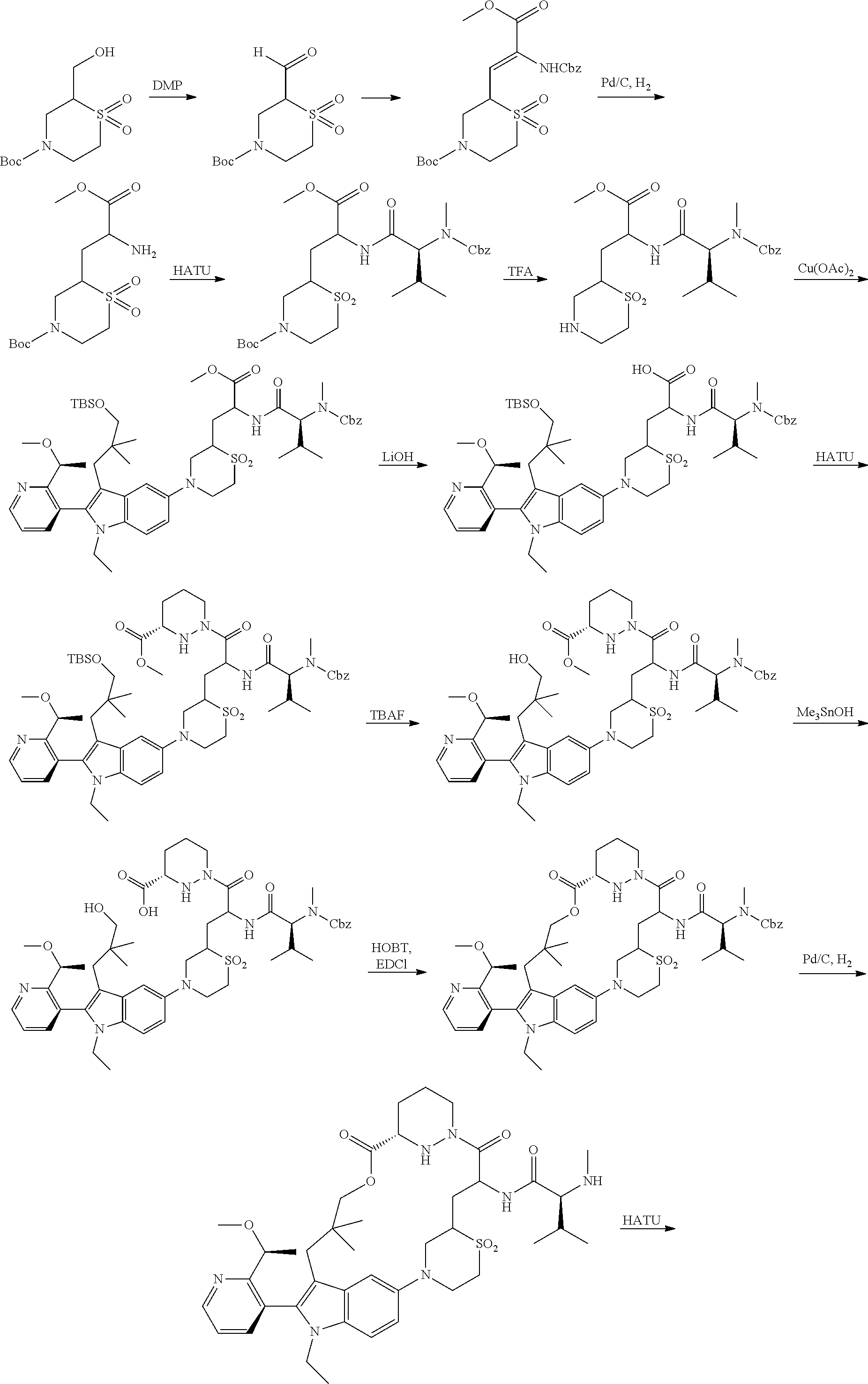

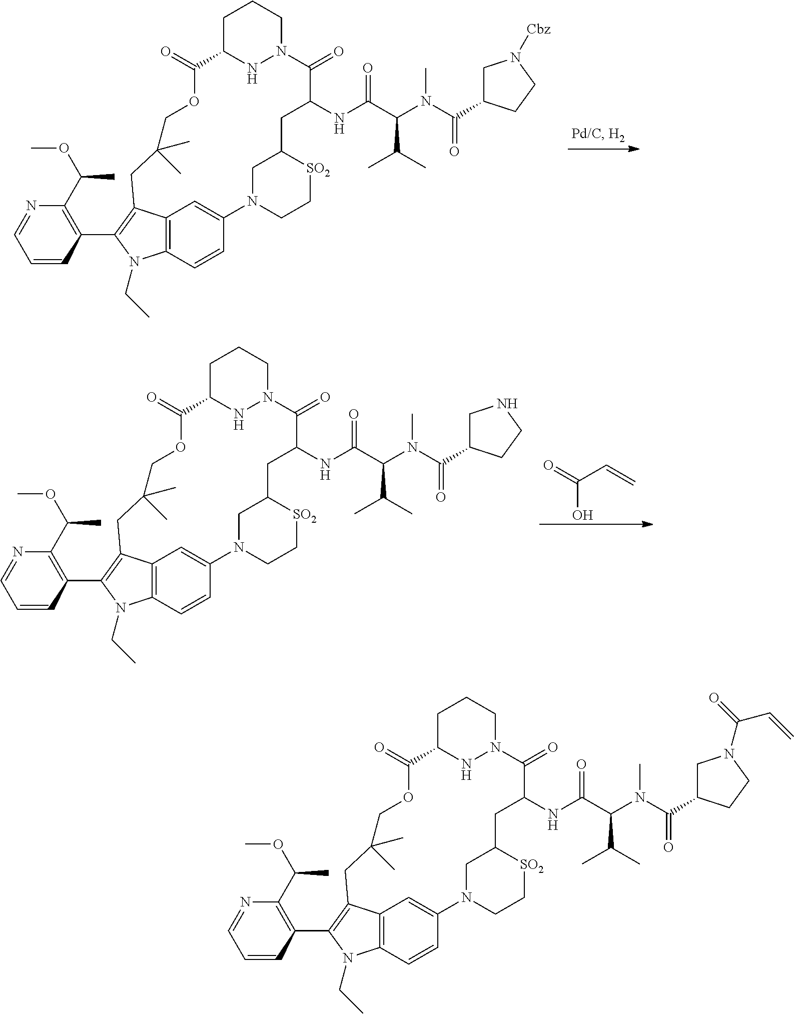

Step 1. To a mixture of tert-butyl 2-(hydroxymethyl)thiomorpholine-4-carboxylate 1,1-dioxide (17.8 g, 60 mmol) in DCM (200 mL) was added Dess-Martin periodinane (56.6 g, 130 mmol). The mixture was stirred at rt for 2 h, then filtered and the filtrate was concentrated under reduced pressure to give tert-butyl 2-formylthiomorpholine-4-carboxylate 1,1-dioxide (30 g) as a syrup, which was used in the next step without further purification. LCMS (ESI): m/z [M−tBu+H]⁺ calc'd for C₆H₉NO₅S 207.2; found 208.0; ¹H NMR (400 MHz, 00013) b 9.88 (s, 1H), 4.17 (d, J=39.4, 33.7 Hz, 4H), 3.15 (d, J=34.2 Hz, 3H), 1.48 (s, 1 OH).

Step 2. To a mixture of tert-butyl 2-formylthiomorpholine-4-carboxylate 1,1-dioxide (58 g, 60 mmol) in ACN (400 mL) at 0° C. was added 1,1,3,3-tetramethylguanidine (30.5 g, 200 mmol) and methyl 2-{[(benzyloxy)carbonyl]amino)-2-(dimethoxyphosphoryl)acetate (43.8 g, 130 mmol). The mixture was warmed to rt and stirred for 2 h then concentrated under reduced pressure. The residue was diluted with EtOAc (200 mL) and washed with H₂O (150 mL×3), then dried and filtered. The filtrate was concentrated under reduced pressure and the residue was purified by silica gel column chromatography to give tert-butyl 2-(2-(((benzyloxy)carbonyl)amino)-3-methoxy-3-oxoprop-1-en-1-yl)thiomorpholine-4-carboxylate 1,1-dioxide (8 g, 25% yield over 2 steps) as a syrup. LCMS (ESI): m/z [M+Na]⁺ calc'd for C₂₁H₂₈N₂NaO₈S 491.2; found 491.2.

Step 3. A mixture of tert-butyl 2-(2-(((benzyloxy)carbonyl)amino)-3-methoxy-3-oxoprop-1-en-1-yl)thiomorpholine-4-carboxylate (8 g, 17.0 mmol), 10% Pd/C (4 g) and NH₄Cl (9.1 g, 170 mmol) in MeOH (200 mL) was stirred at rt under an atmosphere of H₂ for 48 h. The mixture was filtered and the filtrate was concentrated under reduced pressure to give tert-butyl 2-(2-amino-3-methoxy-3-oxopropyl)thiomorpholine-4-carboxylate 1,1-dioxide (6.3 g) as an oil, which was used in next step without further purification. LCMS (ESI): m/z [M+H]⁺ calc'd for C₁₃H₂₄N₂O₆S 336.1; found 337.1.

Step 4. To a mixture of tert-butyl 2-(2-amino-3-methoxy-3-oxopropyl)thiomorpholine-4-carboxylate 1,1-dioxide (6.3 g, 10 mmol) and (2S)-2-({3-[(formyloxy)methyl]phenyl}(methyl)amino)-3-methylbutanoic acid (5 g, 10 mmol) in dry DMF (20 mL) at 0° C. was added DIPEA (49.2 g, 30 mmol) and HATU (7.2 g, 10 mmol). The mixture was stirred at 0° C. for 1 h, then diluted with EtOAc (100 mL) and washed with H₂O (50 mL×3). The combined organic layers were concentrated under reduced pressure and the residue was purified by silica gel column chromatography to give tert-butyl 2-(2-((S)-2-(((benzyloxy)carbonyl)(methyl)amino)-3-methylbutanamido)-3-methoxy-3-oxopropyl)thiomorpholine-4-carboxylate 1,1-dioxide (5 g, 57% yield over 2 steps) as an oil. LCMS (ESI): m/z [M+H]⁺ calc'd for C₂₇H₄₁N₃₀S 583.3; found 584.3.

Step 5. To a mixture of tert-butyl 2-(2-((S)-2-(((benzyloxy)carbonyl)(methyl)amino)-3-methylbutanamido)-3-methoxy-3-oxopropyl)thiomorpholine-4-carboxylate 1,1-dioxide (12 g, 20 mmol) in DCM (80 mL) at 0° C. was added TFA (20 mL). The mixture was warmed to rt and stirred for 1.5 h, then concentrated under reduced pressure. The residue was diluted with EtOAc (50 mL) and adjusted to pH ˜9 with saturated Na₂CO₃. The organic layer was concentrated under reduced pressure to give methyl 2-((S)-2-(((benzyloxy)carbonyl)(methyl)amino)-3-methylbutanamido)-3-(1,1-dioxidothiomorpholin-2-yl)propanoate (9.1 g, yield 94%) as a syrup, which was used in the next step without further purification.

LCMS (ESI): m/z [M+H]⁺ calc'd for C₂₂H₃₃N₃₀₇S 483.2; found 484.2.

Step 6. To a mixture of methyl 2-((S)-2-(((benzyloxy)carbonyl)(methyl)amino)-3-methylbutanamido)-3-(1,1-dioxidothiomorpholin-2-yl)propanoate (5.9 g, 12 mmol) in DCM (50 mL) at rt was added (3-{3-[(tert-butyldimethylsilyl)oxy]-2,2-dimethylpropyl}-1-ethyl-2-{2-[(1S)-1-methoxyethyl]pyridin-3-yl}indol-5-yl)boranediol (6.4 g, 12 mmol), Cu(OAc)₂ (2.2 g, 12 mmol) and pyridine (2.8 g, 36 mmol). The mixture was stirred at rt for 48 h, then the mixture was filtered, the filtrate was diluted with EtOAc (30 mL) and washed with H₂O (80 mL×3). The organic layer was concentrated under reduced pressure and the residue was purified by silica gel column chromatography to give methyl 2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoate (7.59 g, 66% yield) as an oil. LCMS (ESI): m/z [M+H]⁺ calc'd for C₅₁H₇₅N₅O₉SSi 961.5; found 962.3.

Step 7. To a mixture of methyl 2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoate (7.59 g, 7.9 mmol) in THE (40 mL) at 0° C. was added LiOH (0.38 g, 16 mmol) in H₂O (8 mL). The mixture was stirred at 0° C. for 1.5 h, then the pH adjusted to pH ˜7 with 3M HCl (5 mL), the mixture diluted with brine (15 mL) and extracted with EtOAc (50 mL×3). The combined organic layers were concentrated under reduced pressure to give 2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoic acid (7.4 g, 98% yield) as a syrup. LCMS (ESI): m/z [M+H]⁺ calc'd for C₅₀H₇₃N₅O₉SSi 947.5; found 948.4.

Step 8. To a mixture of 2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoic acid (7.4 g, 7.8 mmol) in DMF (50 mL) at 0° C. was added methyl (3S)-1,2-diazinane-3-carboxylate dihydrochloride (2.6 g, 12 mmol), DIPEA (20 g, 160 mmol) and HATU (4.6 g, 12 mmol). The mixture was stirred at 0° C. for 2 h, then diluted with EtOAc (300 mL) and washed with H₂O (100 mL×2). The combined organic layers were concentrated under reduced pressure and the residue was purified by silica gel column chromatography to give (3S)-methyl 1-(2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoyl)hexahydropyridazine-3-carboxylate (8.08 g, 96% yield) as a syrup. LCMS (ESI): m/z [M+H]⁺ calc'd for C₅₆H₈₃N₇O₁₀SSi 1073.6; found 1074.5.

Step 9. To a mixture of 1M TBAF in THE (38 mL, 38 mmol) and AcOH (2.3 g, 38 mmol) was added (3S)-methyl 1-(2-((S)-2-(((benzyloxy)carbonyl)(methyl)amino)-3-methylbutanamido)-3-(4-((R)-3-(3-((tert-butyldimethylsilyl)oxy)-2,2-dimethylpropyl)-1-ethyl-2-(2-((S)-1-methoxyethyl)pyridin-3-yl)-1H-indol-5-yl)-1,1-dioxidothiomorpholin-2-yl)propanoyl)hexahydropyridazine-3-carboxylate (8.08 g, 7.5 mmol). The mixture was heated to 55° C. and stirred for 16 h, then diluted with EtOAc (200 mL) and washed with H₂O (150 mL×2). The combined organic layers were concentrated under reduce pressure to give (3S)-methyl 1-(2-((S)-2-(((benzyloxy)carbonyl)(methyl)amino)-3-methylbutanamido)-3-(4-((R)-1-ethyl-3-(3-hydroxy-2,2-dimethylpropyl)-2-(2-((S)-1-methoxyethyl)pyridin-3-yl)-1H-indol-5-yl)-1,1-dioxidothiomorpholin-2-yl)propanoyl)hexahydropyridazine-3-carboxylate (7.2 g, 99% yield) as a syrup. LCMS (ESI): m/z [M+H]⁺ calc'd for C₅₀H₆₉N₇O₁₀S 959.5; found 960.3.

Step 10. To a mixture of (3S)-methyl 1-(2-((S)-2-(((benzyloxy)carbonyl)(methyl)amino)-3-methylbutanamido)-3-(4-((R)-1-ethyl-3-(3-hydroxy-2,2-dimethylpropyl)-2-(2-((S)-1-methoxyethyl)pyridin-3-yl)-1H-indol-5-yl)-1,1-dioxidothiomorpholin-2-yl)propanoyl)hexahydropyridazine-3-carboxylate (7.2 g, 7.5 mol) in DCE (30 mL) was added Me₃SnOH (6.7 g, 38 mmol). The mixture was heated to 65° C. and stirred for 16 h, then filtered and the filtrate was concentrated under reduced pressure to give (3S)-1-(2-((S)-2-(((benzyloxy)carbonyl)(methyl)amino)-3-methylbutanamido)-3-(4-((R)-1-ethyl-3-(3-hydroxy-2,2-dimethylpropyl)-2-(2-((S)-1-methoxyethyl)pyridin-3-yl)-1H-indol-5-yl)-1,1-dioxidothiomorpholin-2-yl)propanoyl)hexahydropyridazine-3-carboxylic acid (13 g) as an oil. LCMS (ESI): m/z [M+H]⁺ calc'd for C₄₉H₆₇N₇O₁₀S 945.5; found 946.4.

Step 11. To a mixture of (3S)-1-(2-((S)-2-(((benzyloxy)carbonyl)(methyl)amino)-3-methylbutanamido)-3-(4-((R)-1-ethyl-3-(3-hydroxy-2,2-dimethylpropyl)-2-(2-((S)-1-methoxyethyl)pyridin-3-yl)-1H-indol-5-yl)-1,1-dioxidothiomorpholin-2-yl)propanoyl)hexahydropyridazine-3-carboxylic acid (13 g, 7.4 mmol; ca. 55% purity) in DCM (400 mL) at 0° C. was added DIPEA (38 g, 300 mmol), HOBT (10 g, 74 mmol) and EDCI (42 g, 220 mmol). The mixture was warmed to rt and stirred for 48 h, then concentrated under reduced pressure, the residue diluted with EtOAc (200 mL) and washed with H₂O (100 mL×2). The organic layer was concentrated under reduced pressure and the residue was purified by silica gel chromatography to give benzyl ((2S)-1-(((63S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1 (5,3)-indola-6(1,3)-pyridazinacycloundecaphane-4-yl)amino)-3-methyl-1-oxobutan-2-yl)(methyl)carbamate [four isomers; a mixture of Isomer 1 and Isomer 2, 1.6 g; Isomer 3 (651 mg, 9.5% yield); Isomer 4 (332 mg, 4.8% yield)]. The mixture of Isomer 1 and Isomer 2 was purified further by preparative-HPLC to give Isomer 1 (470 mg, 6.8% yield) and Isomer 2 (797 mg, 12% yield).

Data for Isomer 1: LCMS (ESI): m/z [M+H]⁺ calc'd for C₄₉H₆₅N₇O₉S 927.5; found 928.4; ¹H NMR (400 MHz, CD₃OD) δ 8.74 (dd, J=4.8, 1.6 Hz, 1H), 8.36-8.13 (m, 1H), 7.91 (dd, J=7.8, 1.7 Hz, 1H), 7.52 (dd, J=7.8, 4.8 Hz, 1H), 7.45-7.25 (m, 6H), 7.21-7.07 (m, J=8.8 Hz, 1H), 5.59-5.40 (m, 2H), 5.28-5.05 (m, 2H), 4.45 (d, 1H), 4.17 (d, J=11.0 Hz, 1H), 4.13-3.97 (m, 2H), 3.97-3.62 (m, 6H), 3.50-3.34 (m, 2H), 3.27-3.04 (m, 4H), 3.01-2.83 (m, 4H), 2.78 (s, 2H), 2.64-2.32 (m, 2H), 2.24-1.90 (m, 5H), 1.84-1.65 (m, 2H), 1.46 (dd, J=16.6, 6.6 Hz, 3H), 1.36-1.17 (m, 4H), 1.02 (s, 2H), 0.94-0.70 (m, 6H), 0.58 (s, 3H).

Data for Isomer 2: LCMS (ESI): m/z [M+H]⁺ calc'd for C₄₉H₆₅N₇O₉S 927.5; found 928.4; ¹H NMR (400 MHz, CD₃OD) δ 8.71 (dd, J=4.8, 1.6 Hz, 1H), 8.18-8.01 (m, 1H), 7.83 (dd, J=7.7, 1.6 Hz, 1H), 7.52 (dd, J=7.7, 4.9 Hz, 1H), 7.45-7.23 (m, 6H), 7.20 (s, 1H), 7.06 (dd, J=8.9, 2.1 Hz, 1H), 5.66-5.50 (m, 1H), 5.29-5.05 (m, 2H), 4.36-4.18 (m, 3H), 4.17-4.09 (m, 2H), 4.05-3.86 (m, 5H), 3.75 (d, J=16.6 Hz, 1H), 3.54-3.36 (m, 2H), 3.27 (s, 1H), 3.21-3.06 (m, 4H), 3.03-2.91 (m, 1H), 2.88 (s, 3H), 2.81-2.63 (m, 2H), 2.47-2.35 (m, 1H), 2.34-2.09 (m, 3H), 2.00-1.93 (m, 1H), 1.86 (d, J=10.2 Hz, 1H), 1.79-1.63 (m, 2H), 1.43 (d, J=6.2 Hz, 3H), 1.28 (s, 1H), 1.01 (d, J=5.7 Hz, 3H), 0.91-0.77 (m, 10H), 0.57 (s, 3H).

Data for Isomer 3: LCMS (ESI): m/z [M+H]⁺ calc'd for C₄₉H₆₅N₇O₉S 927.5; found 928.4; ¹H NMR (400 MHz, CD₃OD) δ 8.79-8.66 (m, 1H), 8.17-8.04 (m, 1H), 7.88 (dd, J=19.8, 5.4 Hz, 1H), 7.52 (dd, J=7.7, 4.8 Hz, 1H), 7.45-7.16 (m, 7H), 7.15-6.98 (m, 1H), 5.50-5.38 (m, 1H), 5.16 (d, J=8.2 Hz, 2H), 4.32 (d, J=12.0 Hz, 1H), 4.24-4.16 (m, 1H), 4.14-4.02 (m, 2H), 4.00-3.72 (m, 5H), 3.62 (dd, J=30.7, 6.5 Hz, 2H), 3.28-3.14 (m, 2H), 3.11-2.92 (m, 5H), 2.88 (d, J=6.7 Hz, 3H), 2.74-2.54 (m, 1H), 2.52-2.12 (m, 4H), 1.94-1.65 (m, 2H), 1.61-1.47 (m, 1H), 1.43 (d, J=6.3 Hz, 3H), 1.38-1.25 (m, 2H), 1.18 (t, J=6.9 Hz, 3H), 0.98-0.73 (m, 9H), 0.68 (s, 3H).

Data for Isomer 4: LCMS (ESI): m/z [M+H]⁺ calc'd for C₄₉H₆₅N₇O₉S 927.5; found 928.4; ¹H NMR (400 MHz, CD₃OD) δ 8.79-8.61 (m, 1H), 8.21 (d, J=47.9 Hz, 1H), 7.92 (dd, J=7.7, 1.6 Hz, 1H), 7.64-7.46 (m, 2H), 7.44-7.20 (m, 5H), 7.07 (d, J=8.7 Hz, 1H), 5.84-5.45 (m, 1H), 5.26-5.02 (m, 2H), 4.42-3.38 (m, 11H), 3.27-3.06 (m, 4H), 3.05-2.94 (m, 3H), 2.93-2.70 (m, 4H), 2.53 (t, 1H), 2.27-2.09 (m, 2H), 2.01 (d, J=3.8 Hz, 1H), 1.87-1.54 (m, 3H), 1.52-1.26 (m, 3H), 1.26-0.98 (m, 4H), 0.97-0.40 (m, 12H).

Step 12. A mixture of benzyl ((2S)-1-(((63S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)(methyl)carbamate (Isomer 1; 380 mg, 0.41 mmol), Pd/C, 50% wt with H₂O (100 mg) and NH₄Cl (220 mg, 4.1 mmol) in MeOH (10 mL), was stirred at 15° C. for 10 h. The mixture was filtered, the filtrate was concentrated under reduced pressure, the residue was diluted with sat. NaHCO₃ (20 mL) and extracted with DCM (20 mL×5). The combined organic layers was dried over Na₂SO₄, filtered and the filtrate was concentrated under reduced pressure to give (2S)—N-((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)-3-methyl-2-(methylamino)butanamide (300 mg, 92% yield) as a solid, and used in the next step without further purification. LCMS (ESI): m/z [M+H]⁺ calc'd for C₄₁H₅₉N₇O₇S 793.4; found 794.4.

A similar reaction was undertaken using Isomers 2, 3 and 4 as starting material to give the respective products.

Data for Isomer 2: Starting from (170 mg, 0.18 mmol) to give (140 mg, 98% yield). LCMS (ESI): m/z [M+H]⁺ calc'd for C₄₁H₅₉N₇O₇S 793.4; found 794.4.

Data for Isomer 3: Starting from (390 mg, 0.42 mmol) to give (300 mg, 90% yield). LCMS (ESI): m/z [M+H]⁺ calc'd for C₄₁H₅₉N₇O₇S 793.4; found 794.3.

Data for Isomer 4: Starting from (240 mg, 0.26 mmol) to give (200 mg, 96% yield). LCMS (ESI): m/z [M+H]⁺ calc'd for C₄₁H₅₉N₇O₇S 793.4; found 794.3.

Step 13. To a mixture of (2S)—N-((6³S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)-3-methyl-2-(methylamino)butanamide (Isomer 1; 120 mg, 0.15 mmol) and (3S)-1-{3-[(formyloxy)methyl]phenyl}pyrrolidine-3-carboxylic acid (56 mg, 0.23 mmol) in DMF (5 mL) at 0° C. was added DIPEA (390 mg, 3 mmol) and HATU (87 mg, 0.23 mmol). The mixture was stirred at 0° C. for 1 h, then diluted with EtOAc (20 mL) and washed with H₂O (20 mL×2). The organic layer was dried over Na₂SO₄, filtered, the filtrate was concentrated under reduced pressure and the residue was purified by silica gel chromatography to give benzyl (3S)-3-(((2S)-1-(((63S)-1¹-ethyl-1²-(2-((S)-1-methoxyethyl)pyridin-3-yl)-10,10-dimethyl-2,2-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)(methyl)carbamoyl)pyrrolidine-1-carboxylate (111 mg, 72% yield) as a solid. LCMS (ESI): m/z [M+H]⁺ calc'd for C₅₄H₇₂N₈O₁₀S 1024.5; found 1025.3.

A similar reaction was undertaken using Isomers 2, 3 and 4 as starting material to give the respective products.

Data Isomer 2: Starting from (150 mg, 0.19 mmol) to give (120 mg, 62% yield). LCMS (ESI): m/z [M+H]⁺ calc'd for C₅₄H₇₂N₈O₁₀S 1024.5; found 1025.4.

Data for Isomer 3: Starting from (300 mg, 0.38 mmol) to give (300 mg, 77% yield). LCMS (ESI): m/z [M+H]⁺ calc'd for C₅₄H₇₂N₈O₁₀S 1024.5; found 1025.5.

Data for Isomer 4: Starting from (199 mg, 0.25 mmol) to give (220 mg, 85% yield). LCMS (ESI): m/z [M+H]⁺ calc'd for C₅₄H₇₂N₈O₁₀S 1024.5; found 1025.4.

Step 14. A mixture of benzyl (3S)-3-(((2S)-1-(((63S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)(methyl)carbamoyl)pyrrolidine-1-carboxylate (Isomer 1; 111 mg, 0.11 mmol), Pd/C, 50% wt. with H₂O (30 mg) and NH₄Cl (60 mg, 1.1 mmol) in MeOH (20 mL) was stirred at 15° C. for 10 h. The mixture was filtered, the filtrate was concentrated under reduced pressure and the residue was diluted with DCM (20 mL) and washed with sat. NaHCO₃. The organic layer was dried over Na₂SO₄, filtered and the filtrate was concentrated under reduced pressure to give (3S)—N-((2S)-1-(((63S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)-N-methylpyrrolidine-3-carboxamide (77 mg, 79% yield) as a solid, which was used in the next step without further purification. LCMS (ESI): m/z [M+H]⁺ calc'd for C₄₆H₆₆N₈O₈S 890.5; found 891.4.

A similar reaction was undertaken using Isomers 2, 3 and 4 as starting material to give the respective products.

Data for Isomer 2: Starting from (120 mg, 0.12 mmol) to give (85 mg, 89% yield). LCMS (ESI): m/z [M+H]⁺ calc'd for C₄₆H₆₆N₈O₈S 890.5; found 891.4.

Data for Isomer 3: Starting from (300 mg, 0.34 mmol) to give (220 mg, 73% yield). LCMS (ESI): m/z [M+H]⁺ calc'd for C₄₆H₆₆N₈O₈S 890.5; found 891.5.

Data for Isomer 4: Starting from (220 mg, 0.21 mmol) to give (147 mg, 71% yield). LCMS (ESI): m/z [M+H]⁺ calc'd for C₄₆H₆₆N₈O₈S 890.5; found 891.4.

Step 15. To a mixture of (3S)—N-((2S)-1-(((63S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)-N-methylpyrrolidine-3-carboxamide (Isomer 1; 77 mg, 0.086 mmol) in DCM (2 mL) at 0° C. was added sat. NaHCO₃ (2 mL) and prop-2-enoyl chloride (7 mg, 0.077 mmol) in DCM (1 mL). The mixture was stirred at 0° C. for 30 min, then H₂O added and the mixture extracted with DCM (10 mL×3). The combined organic layers were dried over Na₂SO₄, filtered, the filtrate was concentrated under reduced pressure and the residue was purified by preparative-TLC to give (3S)-1-acryloyl-N-((2S)-1-(((63S)-1¹-ethyl-1²-(2-((S)-1-methoxyethyl)pyridin-3-yl)-10,10-dimethyl-2¹,2¹-dioxido-5,7-dioxo-6¹,6²,6³,6⁴,6⁵,6⁶-hexahydro-1¹H-8-oxa-2(4,2)-thiomorpholina-1(5,3)-indola-6(1,3)-pyridazinacycloundecaphane-4-yl)amino)-3-methyl-1-oxobutan-2-yl)-N-methylpyrrolidine-3-carboxamide (23 mg, 28% yield) as a solid. LCMS (ESI): m/z [M+H]⁺ calc'd for C₄₉H₆₈N₈O₉S 944.5; found 945.4; ¹H NMR (400 MHz, CD₃OD) δ 8.75-8.74 (m, 1H), 7.92-7.90 (m, 1H), 7.54-7.51 (m, 1H), 7.43 (dd, J=8.8, 2.2 Hz, 1H), 7.34 (d, J=3.2 Hz, 1H), 7.25-7.15 (m, 1H), 6.71-6.60 (m, 1H), 6.32-6.25 (m, 1H), 5.77 (dd, J=10.5, 1.9 Hz, 1H), 5.53-5.48 (m, 1H), 4.62 (dd, J=24.9, 11.1 Hz, 1H), 4.45 (s, 1H), 4.13-4.03 (m, 3H), 3.89-3.76 (m, 6H), 3.69-3.63 (m, 2H), 3.60-3.35 (m, 3H), 3.25-3.21 (m, 3H), 3.13-3.11 (m, 1H), 3.00 (d, J=2.3 Hz, 5H), 2.90 (d, J=3.5 Hz, 2H), 2.25-2.20 (m, 2H), 2.16-2.09 (m, 3H), 2.04-1.94 (m, 2H), 1.80-1.72 (m, 2H), 1.46-1.43 (m, 3H), 1.29 (m, 3H), 1.26-1.22 (m, 3H), 1.01-0.98 (m, 3H), 0.95-0.88 (m, 3H), 0.84-0.81 (m, 3H), 0.62-0.59 (m, 2H).

A similar reaction was undertaken using Isomers 2, 3 and 4 as starting material to give the respective products.

Data for Isomer 2: Starting from (110 mg, 0.12 mmol) to give (24.5 mg, 21% yield). LCMS (ESI): m/z [M+H]⁺ calc'd for C₄₉H₆₈N₈O₉S 944.5; found 945.3; ¹H NMR (400 MHz, CD₃OD) δ 8.71 (dd, J=4.8, 1.7 Hz, 1H), 7.91-7.78 (m, 1H), 7.52 (dd, J=7.7, 4.9 Hz, 1H), 7.45-7.36 (m, 1H), 7.25-7.03 (m, 2H), 6.65-6.56 (m, 1H), 6.30-6.22 (m, 1H), 5.76-5.70 (m, 1H), 5.67-5.48 (m, 1H), 5.27 (dd, J=11.7, 8.2 Hz, 1H), 4.69 (dd, J=10.9, 3.3 Hz, 1H), 4.37-4.28 (m, 1H), 4.26-4.18 (m, 1H), 4.18-3.98 (m, 3H), 3.97-3.83 (m, 4H), 3.82-3.62 (m, 4H), 3.60-3.41 (m, 3H), 3.28-3.20 (m, 2H), 3.14 (d, J=10.4 Hz, 3H), 3.06 (d, J=4.8 Hz, 3H), 2.96 (s, 1H), 2.89-2.77 (m, 1H), 2.73-2.55 (m, 1H), 2.48-2.34 (m, 1H), 2.33-2.18 (m, 3H), 2.13-1.95 (m, 2H), 1.90-1.84 (m, 1H), 1.80-1.67 (m, 2H), 1.43 (m, 3H), 1.27 (s, 1H), 1.14-0.95 (m, 4H), 0.94-0.85 (m, 4H), 0.82 (d, J=6.2 Hz, 5H), 0.56 (d, J=8.7 Hz, 3H).

Data for Isomer 3: Starting from (120 mg, 0.13 mmol) to give (32 mg, 11% yield). LCMS (ESI): m/z [M+H]⁺ calc'd for C₄₉H₆₈N₈O₉S 944.5; found 945.5; ¹H NMR (400 MHz, CD₃OD) δ 8.73 (dt, J=3.8, 1.9 Hz, 1H), 7.93-7.86 (m, 1H), 7.53 (dd, J=7.7, 4.9 Hz, 1H), 7.40 (dd, J=8.8, 2.3 Hz, 1H), 7.28 (d, J=9.6 Hz, 1H), 7.13-6.99 (m, 1H), 6.65 (ddd, J=35.6, 16.8, 10.5 Hz, 1H), 6.28 (ddd, J=16.8, 4.9, 1.9 Hz, 1H), 5.75 (td, J=10.4, 1.9 Hz, 1H), 5.53-5.34 (m, 1H), 4.63 (dd, J=13.4, 11.3 Hz, 1H), 4.26 (d, J=11.1 Hz, 1H), 4.12-4.01 (m, 2H), 4.00-3.82 (m, 5H), 3.82-3.45 (m, 7H), 3.41-3.33 (m, 1H), 3.14-3.02 (m, 4H), 3.02-2.87 (m, 5H), 2.62-2.34 (m, 3H), 2.33-2.17 (m, 3H), 2.10-1.94 (m, 1H), 1.69-1.52 (m, 1H), 1.46-1.39 (m, 3H), 1.27 (s, 2H), 1.23-1.16 (m, 3H), 1.16-1.01 (m, 2H), 0.96-0.90 (m, 3H), 0.88-0.74 (m, 6H), 0.73-0.63 (m, 3H).

Data for Isomer 4: Starting from (147 mg, 0.16 mmol) to give (47.2 mg, 31% yield). LCMS (ESI): m/z [M+H]⁺ calc'd for C₄₉H₆₈N₈O₉S 944.5; found 945.3; ¹H NMR (400 MHz, CD₃OD) δ 8.73-8.72 (m, 1H), 7.92 (dd, J=7.8, 1.6 Hz, 1H), 7.53-7.50 (m, 1H), 7.49-7.46 (m, 1H), 7.41-7.38 (m, 1H), 7.07 (d, J=8.8 Hz, 1H), 6.65-6.56 (m, 1H), 6.28-6.23 (m, 1H), 5.76-5.71 (m, 2H), 4.59-4.55 (m, 1H), 4.34-4.30 (m, 1H), 4.13-4.03 (m, 4H), 3.88-3.72 (m, 6H), 3.68-3.48 (m, 5H), 3.30-3.20 (m, 4H), 3.08-3.07 (m, 3H), 3.02 (d, J=4.1 Hz, 4H), 2.55-2.53 (m, 1H), 2.34-2.19 (m, 3H), 2.11-2.00 (m, 3H), 1.90-1.88 (m, 1H), 1.76-1.74 (m, 2H), 1.44 (d, J=6.3 Hz, 3H), 1.29 (s, 1H), 1.23-1.20 (m, 3H), 0.91-0.86 (m, 3H), 0.78-0.75 (m, 5H), 0.69-0.66 (m, 3H).

### Synthesis of 3-acryloyl-N-((2S)-1-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N,1-dimethyl-1,3,8-triazaspiro[4.5]decane-8-carboxamide

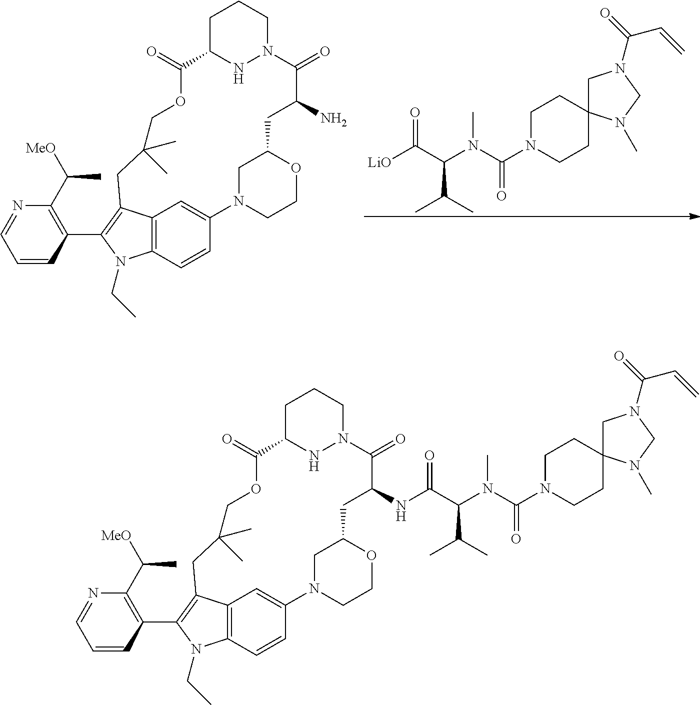

To a mixture of (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (150 mg, 0.24 mmol) and (2S)-3-methyl-2-{methyl[1-methyl-3-(prop-2-enoyl)-1,3,8-triazaspiro[4.5]decan-8-yl]carbonylamino}butanoate, lithium salt (132 mg, 0.36 mmol) in DMF (5 mL) at 0° C. was added HATU (108 mg, 0.28 mmol) and DIPEA (459 mg, 3.5 mmol). The mixture was stirred at 0° C. for 1 h, then diluted with EtOAc (30 mL), washed with H₂O (10 mL×2) and brine (10 mL). The organic layer was dried over Na₂SO₄ and filtered. The filtrate was concentrated under reduced pressure and the crude residue was purified by silica gel column chromatography and preparative-HPLC to give 3-acryloyl-N-((2S)-1-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N,1-dimethyl-1,3,8-triazaspiro[4.5]decane-8-carboxamide (6.9 mg, 3% yield) as a solid. LCMS (ESI): m/z [M+H]⁺ calc'd for C₅₃H₇₆N₁₀O₈ 980.6; found 367.2; ¹H NMR (400 MHz, CD₃OD) δ 8.71 (dd, J=4.8, 1.6 Hz, 1H), 7.86 (dd, J=7.8, 1.6 Hz, 1H), 7.51 (dd, J=7.8, 4.8 Hz, 1H), 7.39 (d, J=8.8 Hz, 1H), 7.14-7.04 (m, 2H), 6.67-6.44 (m, 1H), 6.31 (d, J=16.8 Hz, 1H), 5.81-5.75 (m, 1H), 5.65 (d, J=9.0 Hz, 1H), 4.51-4.13 (m, 2H), 4.33 (s, 1H), 4.27-4.18 (m, 1H), 4.17-4.08 (m, 1H), 3.96-3.87 (m, 3H), 3.87-3.77 (m, 3H), 3.76-3.65 (m, 4H), 3.64-3.51 (m, 3H), 3.28-3.24 (m, 1H), 3.16 (s, 3H), 3.10-3.02 (m, 1H), 2.99-2.90 (m, 2H), 2.87-2.74 (m, 5H), 2.70-2.53 (m, 2H), 2.40-2.30 (m, 3H), 2.27-2.18 (m, 1H), 2.14-2.05 (m, 2H), 1.98-1.88 (m, 3H), 1.79-1.68 (m, 2H), 1.65-1.47 (m, 3H), 1.44 (d, J=6.4 Hz, 3H), 1.04 (t, J=6.8 Hz, 3H), 0.95 (d, J=6.4 Hz, 3H), 0.88 (d, J=6.4 Hz, 3H), 0.80-0.60 (m, 6H).

### Synthesis 2-acryloyl-N-((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)-N-methyl-2,8-diazaspiro[4.5]decane-8-carboxamide

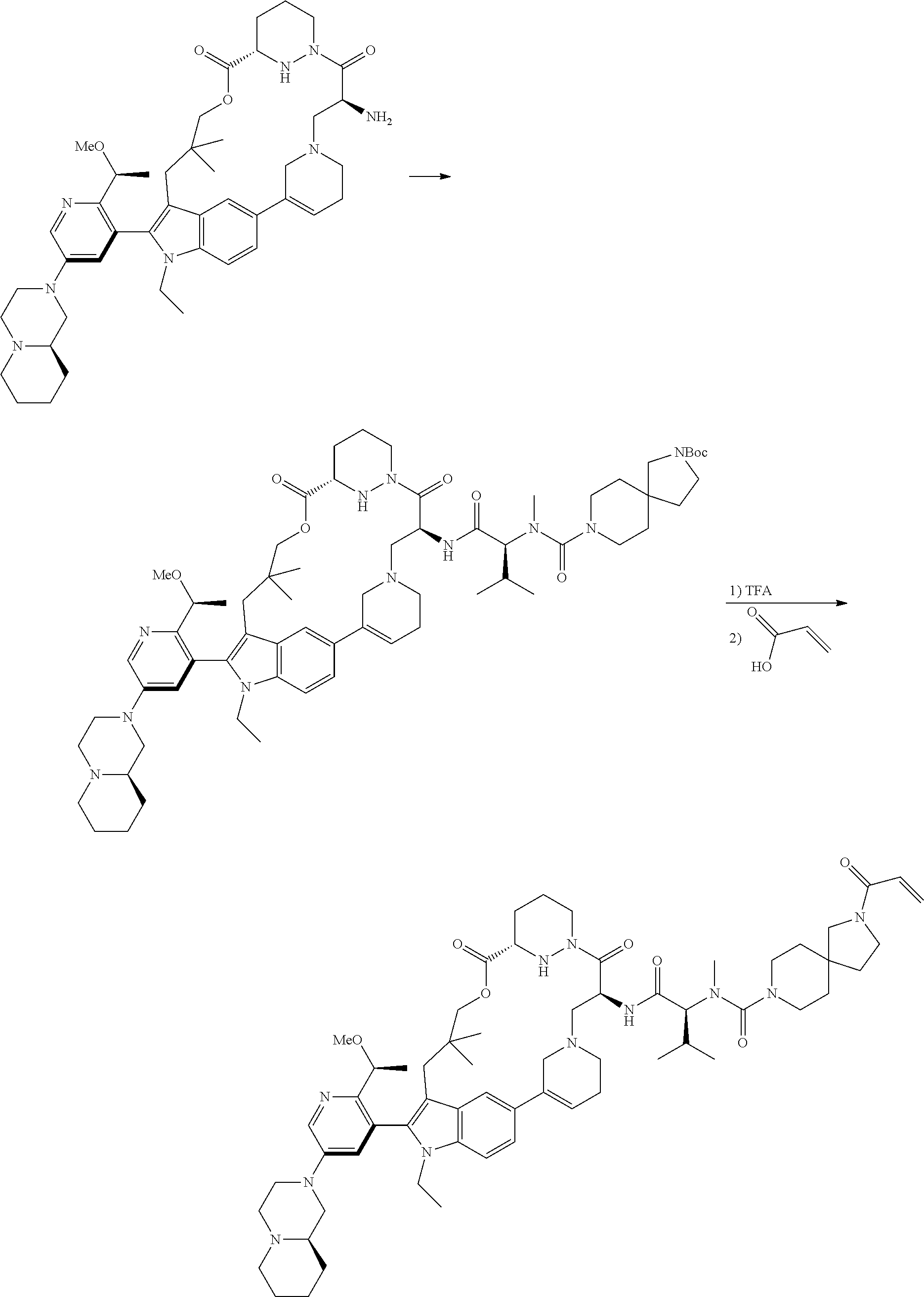

Step 1. To a mixture of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-2¹,2²,2³,2⁶,6¹,6²,6³,6⁴,6⁵,6⁶-decahydro-1′H-8-oxa-1(5,3)-indola-6(1,3)-pyridazina-2(5,1)-pyridinacycloundecaphane-5,7-dione (150 mg, 0.23 mmol) in DMF (2 mL) at 0° C. was added (2S)-2-({2-[(tert-butoxy)carbonyl]-2,8-diazaspiro[4.5]decan-8-yl}carbonyl(methyl)amino)-3-methylbutanoic acid (125 mg, 0.30 mmol), DIPEA (310 mg, 2.34 mmol) and HATU (134 mg, 0.35 mmol). The mixture was stirred at 0° C. for 1 h, then H₂O (150 mL) and extracted with EtOAc (150 mL×2). The combined organic layers were washed with H₂O (50 mL), brine (50 mL), dried over anhydrous Na₂SO₄ and filtered. The filtrate was concentrated under reduced pressure and the crude residue was purified by preparative-TLC to give tert-butyl 8-(((2S)-1-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)amino)-3-methyl-1-oxobutan-2-yl)(methyl)carbamoyl)-2,8-diazaspiro[4.5]decane-2-carboxylate (130 mg, 40% yield) as a solid. LCMS (ESI): m/z [M+H]⁺ calc'd for C₆₄H₉₅N₁₁O₈ 1145.7; found 1146.7.

Step 2. To a mixture of tert-butyl 8-(((2S)-1-(((63S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)(methyl)carbamoyl)-2,8-diazaspiro[4.5]decane-2-carboxylate (130 mg, 0.12 mmol) in DCM (1.0 mL) at 0° C. was added TFA (0.5 mL). The mixture was stirred at 0° C. for 1 h, then diluted with DCM (5 mL) and saturated NaHCO₃ added to adjust pH ˜9. Prop-2-enoyl chloride (10 mg, 0.11 mmol) in DCM was added at 0° C., and the mixture was stirred at 0° C. for 15 min. The mixture was poured into H₂O (50 mL) and extracted with DCM (150 mL×2). The combined organic layers were washed with H₂O (50 mL), brine (50 mL), dried over anhydrous Na₂SO₄ and filtered. The filtrate was concentrated under reduced pressure and the crude residue was purified by preparative-TLC to give 2-acryloyl-N-((2S)-1-(((63S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)- pyridazina-2(5,1)-pyridinacycloundecaphane-4-yl)amino)-3-methyl-1-oxobutan-2-yl)-N-methyl-2,8-diazaspiro[4.5]decane-8-carboxamide as a solid. LCMS (ESI): m/z [M+H]⁺ calc'd for C₆₂H₈₉N₁O₇ 1099.7; found 1100.6; ¹H NMR (400 MHz, CD₃OD) δ 8.45 (d, J=2.8 Hz, 1H), 7.54 (d, J=9.2 Hz, 2H), 7.42 (d, J=8.4 Hz, 2H), 6.65 (m, 1H), 6.41-6.21 (m, 2H), 5.93 (dd, J=7.6, 3.8 Hz, 1H), 5.81-5.75 (m, 1H), 4.50 (d, J=12.8 Hz, 1H), 4.20-4.04 (m, 3H), 3.98-3.71 (m, 8H), 3.63-3.48 (m, 2H), 3.46-3.36 (m, 2H), 3.30-3.15 (m, 3H), 3.12-2.97 (m, 6H), 2.93-2.76 (m, 6H), 2.64 (t, J=11.2 Hz, 2H), 2.55 (d, J=11.6 Hz, 9H), 2.45-2.12 (m, 4H), 1.99-1.83 (m, 2H), 1.80-1.55 (m, 10H), 1.48-1.29 (m, 6H), 1.22 (t, J=7.0 Hz, 3H), 0.93 (dd, J=22.8, 6.4 Hz, 9H), 0.72 (s, 3H).

### Synthesis of 3-acryloyl-N-((2S)-1-(((2²S,6³S,4S)-1¹-ethyl-12-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N-methyl-1-oxa-3,8-diazaspiro[4.5]decane-8-carboxamide

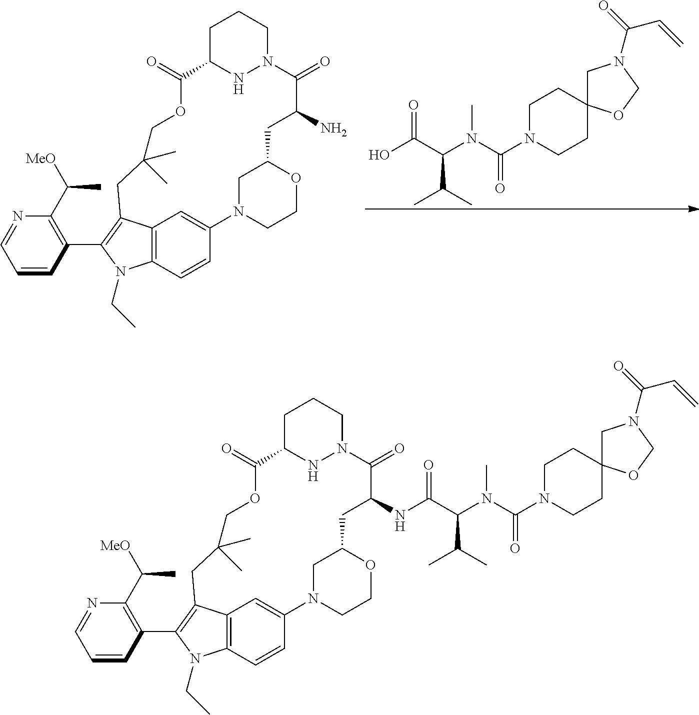

To a mixture of (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (450 mg, 0.7 mmol). The mixture was stirred at 0° C. for 1 h, then H₂O (20 mL) was added and the mixture was extracted with EtOAc (30 mL×3). The combined organic layers were washed with brine (20 mL), dried over anhydrous Na₂SO₄ and filtered. The filtrate was concentrated under reduced pressure and the crude residue was purified by silica gel column chromatography and preparative-HPLC to give 3-acryloyl-N-((2S)-1-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-3-methyl-1-oxobutan-2-yl)-N-methyl-1-oxa-3,8-diazaspiro[4.5]decane-8-carboxamide (297 mg, 40% yield) as a solid. LCMS (ESI): m/z [M+H]⁺ calc'd for C₅₂H₇₃N₉O₉ 967.6; found 968.6; ¹H NMR (400 MHz, CD₃OD) δ 8.72-8.69 (m, 1H), 8.10 (d, J=6.4 Hz, 1H), 7.89-7.80 (m, 1H), 7.56-7.47 (m, 1H), 7.45-7.35 (m, 1H), 7.17-7.01 (m, 2H), 6.62-6.45 (m, 1H), 6.32 (s, 1H), 5.85-5.71 (m, 1H), 5.64 (d, J=8.8 Hz, 1H), 5.19 (s, 1H), 5.10 (s, 1H), 4.46 (d, J=12.4 Hz, 1H), 4.25-4.03 (m, 2H), 3.99-3.61 (m, 8H), 3.61-3.33 (m, 6H), 3.29-3.18 (m, 2H), 3.15 (s, 3H), 2.99-2.71 (m, 6H), 2.68-2.46 (m, 2H), 2.30-2.17 (m, 1H), 2.12-2.02 (m, 2H), 1.96-1.54 (m, 8H), 1.43 (d, J=6.4 Hz, 3H), 1.15-0.97 (m, 3H), 0.96-0.79 (m, 6H), 0.77-0.53 (m, 6H).

### Synthesis of 4-acryloyl-N-((2S)-1-(((63S,4S)-1¹-ethyl-12-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)amino)-3-methyl-1-oxobutan-2-yl)-N-methyl-1-propyl-1,4,9-triazaspiro[5.5]undecane-9-carboxamide

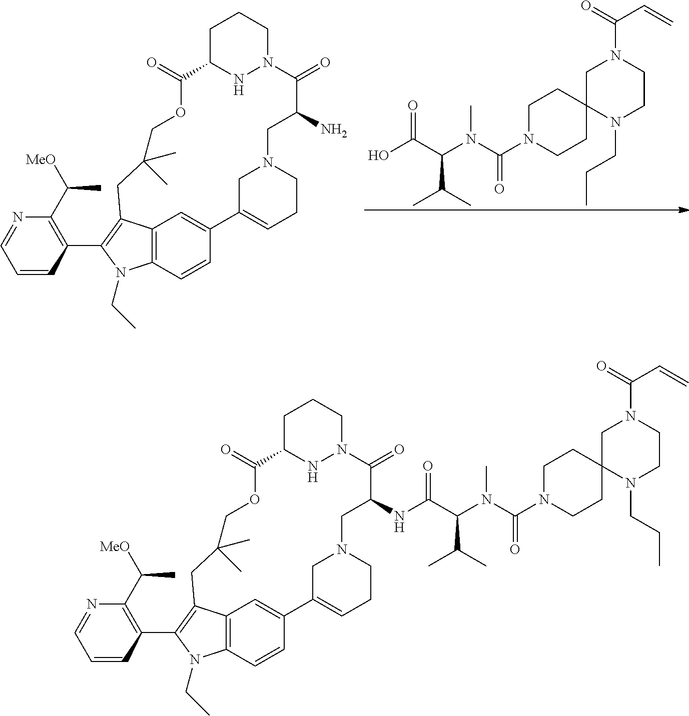

To a mixture of (6³S,4S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1 (5,3)-indola-6(1,3)-pyridazina-2(5,1)- pyridinacycloundecaphane-5,7-dione (113 mg, 0.18 mmol) and (2S)-3-methyl-2-{methyl[4-(prop-2-enoyl)-1-propyl-1,4,9-triazaspiro[5.5]undecan-9-yl]carbonylamino}butanoic acid, lithium salt (88 mg, 0.22 mmol) in DMF (2 mL) at 0° C. was added DIPEA (464 mg, 3.6 mmol) and HATU (82 mg, 0.23 mmol). The mixture was stirred at 0° C. for 1 h, then H₂O (20 mL) was added and the mixture was extracted with DCM (20 mL×3). The combined organic layers were washed with brine (20 mL), dried over anhydrous Na₂SO₄ and filtered. The filtrate was concentrated under reduced pressure and the crude residue was purified by preparative-HPLC to give 4-acryloyl-N-((2S)-1-(((6³S,4S)-1¹-ethyl-1²-(2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1 (5,3)-indola-6(1,3)- pyridazina-2(5,1)-pyridinacycloundecaphane-4-yl)amino)-3-methyl-1-oxobutan-2-yl)-N-methyl-1-propyl-1,4,9-triazaspiro[5.5]undecane-9-carboxamide (26 mg, 14% yield) as a solid. LCMS (ESI): m/z [M+H]⁺ calc'd for C₅₇H₈₂N₁₀O₇ 1018.6; found 1019.6; ¹H NMR (400 MHz, CD₃OD) δ 8.73 (dd, J=8.0, 4.0 Hz, 1H), 7.90 (dd, J=8.0, 4.0 Hz, 1H), 7.54-7.51 (m, 3H), 7.41-7.38 (m, 1H), 6.90-6.74 (m, 1H), 6.30-6.18 (m, 2H), 5.91-5.88 (m, 1H), 5.80-5.75 (m, 1H), 4.59-4.46 (m, 1H), 4.10-3.47 (m, 15H), 3.19-2.72 (m, 17H), 2.42-2.15 (m, 8H), 2.08-1.63 (m, 7H), 1.48-1.44 (m, 6H), 1.16 (t, J=6.4 Hz, 3H), 0.93-0.86 (m, 9H), 0.66 (s, 3H).

### Synthesis of (2S)—N-[(6S,8S,14S,20M)-21-[5-(4-cyclopropylpiperazin-1-yl)-2-[(18)-1-methoxyethyl]pyridin-3-yl]-22-ethyl-18,18-dimethyl-9,15-dioxo-5,16-dioxa-2,10,22,28-tetraazapentacyclo[18.5.2.1^{2,6}0.1^{10,14}0.0^{23,27}]nonacosa-1(26),20,23(27),24-tetraen-8-yl]-3-methyl-2-{methyl[3-(prop-2-enoyl)-1-oxa-3,8-diazaspiro[4.5]decane-8-carbonyl]amino}butanamide

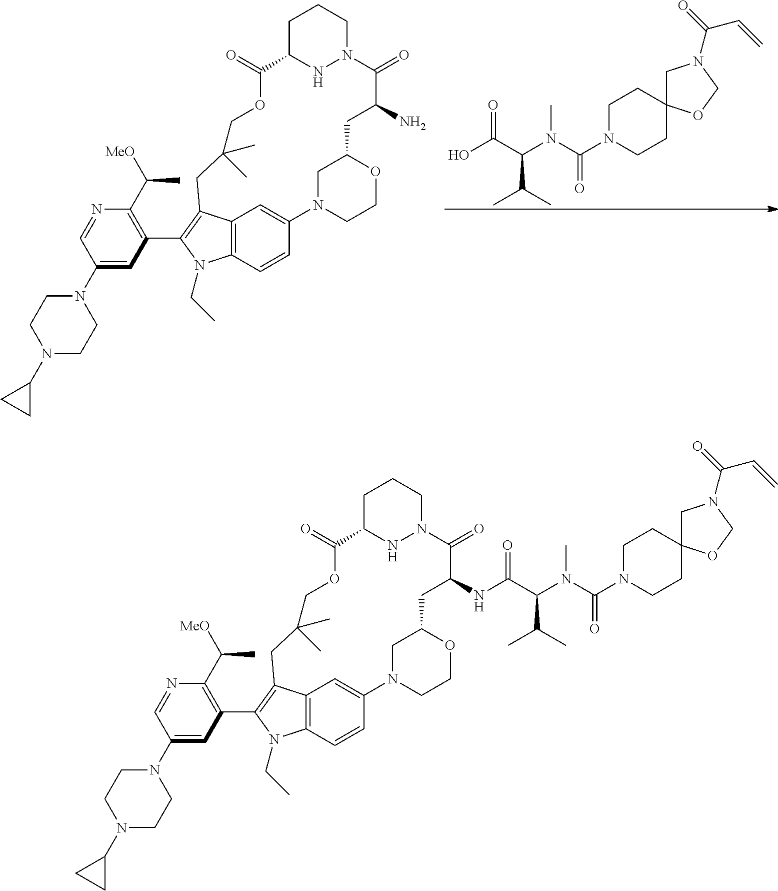

To a solution of ((6S,8S,14S)-8-amino-21-[5-(4-cyclopropylpiperazin-1-yl)-2-[(1S)-1-methoxyethyl]pyridin-3-yl]-22-ethyl-18,18-dimethyl-5,16-dioxa-2,10,22,28-tetraazapentacyclo[18.5.2.1\hat{ }{2,6}0.1\hat{ }{10,14}0.0\hat{ }{23,27}]nonacosa-1(26),20,23(27),24-tetraene-9,15-dione (60 mg, 0.08 mmol, 1 equiv) and (2S)-3-methyl-2-{methyl[3-(prop-2-enoyl)-1-oxa-3,8-diazaspiro[4.5]decan-8-yl]carbonylamino}butanoic acid (42 mg, 0.119 mmol, 1.5 equiv,) in DMF (3 mL) was added N,N-Diisopropylethylamine (205 mg, 1.59 mmol, 20 equiv) followed by HATU (60 mg, 0.159 mmol, 2 equiv) at −5˜0° C. This reaction was stirred at −5˜0° C. for 1 h. The reaction mixture was quenched with water (5 mL) and extracted with EA (10 mL×3). The combined organic phase was washed with water (10 mL×1) and brine (10 mL×1). The organic phase was concentrated to dryness and the resulting residue was purified by chromatography to afford (2S)—N-[(6S,8S,14S,20M)-21-[5-(4-cyclopropylpiperazin-1-yl)-2-[(1S)-1-methoxyethyl]pyridin-3-yl]-22-ethyl-18,18-dimethyl-9,15-dioxo-5,16-dioxa-2,10,22,28-tetraazapentacyclo[18.5.2.1^{2,6}0.1^{10,14}0.0^{23,27}]nonacosa-1(26),20,23(27),24-tetraen-8-yl]-3-methyl-2-{methyl[3-(prop-2-enoyl)-1-oxa-3,8-diazaspiro[4.5]decane-8-carbonyl]amino}butanamide (16 mg, 18% yield) as a white solid. ESI-MS m/z=1092.6[M+H]⁺; Calculated MW: 1091.7; ¹H NMR (400 MHz, CD₃OD) δ 8.29 (d, J=2.9 Hz, 1H), 7.25 (dd, J=22.5, 5.9 Hz, 2H), 7.06-6.91 (m, 2H), 6.51-6.16 (m, 2H), 5.75-5.63 (m, 1H), 5.55 (d, J=8.9 Hz, 1H), 5.09 (d, J=5.0 Hz, 1H), 5.00 (s, 1H), 4.57-4.32 (m, 2H), 4.09-3.95 (m, 2H), 3.98-3.50 (m, 9H), 3.52-3.21 (m, 7H), 3.23-3.03 (m, 8H), 3.03 (s, 3H), 2.85 (dd, J=26.6, 15.7 Hz, 2H), 2.69 (d, J=14.8 Hz, 2H), 2.61-2.41 (m, 2H), 2.22-2.07 (m, 1H), 1.99 (dd, J=18.3, 12.0 Hz, 2H), 1.94-1.09 (m, 13H), 0.98 (t, J=6.9 Hz, 3H), 0.81 (dd, J=27.0, 6.5 Hz, 6H), 0.64 (d, J=28.9 Hz, 6H), 0.50-0.32 (m, 4H).

### Synthesis of (2S)—N-[(8S,14S,20M)-22-ethyl-21-{2-[(1S)-1-methoxyethyl]-5-(4-methylpiperazin-1-yl)pyridin-3-yl}-18,18-dimethyl-9,15-dioxo-16-oxa-6,10,22,28-tetraazapentacyclo[18.5.2.1^{2,6}0.1^{10,14}0.0^{23,27}]nonacosa-1(26),2,20,23(27),24-pentaen-8-yl]-3-methyl-2-{methyl[3-(prop-2-enoyl)-1-oxa-3,8-diazaspiro[4.5]decane-8-carbonyl]amino}butanamide

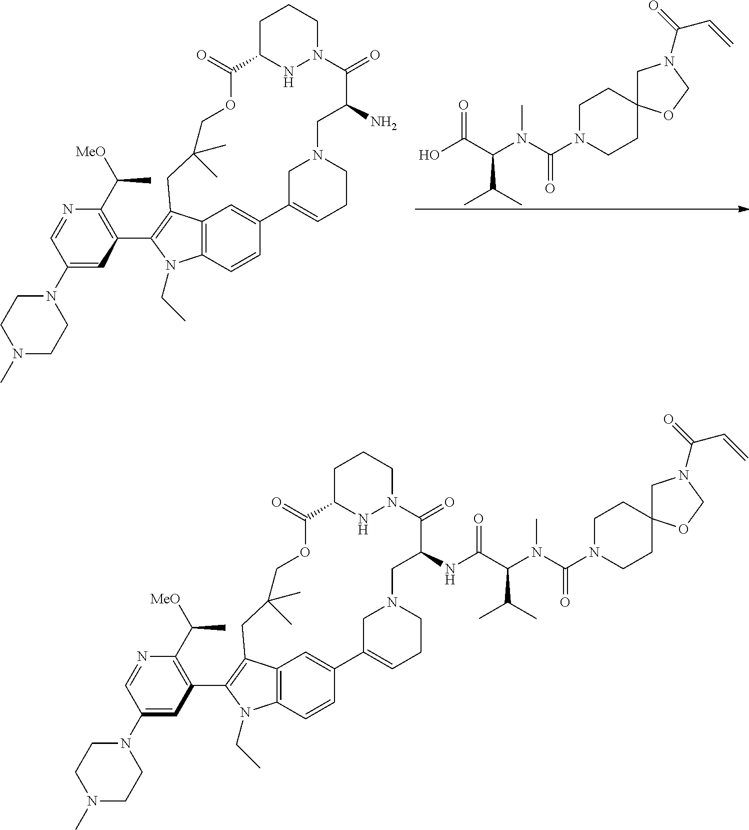

To a solution of (8S)-8-amino-22-ethyl-21-{2-[(1S)-1-methoxyethyl]-5-(4-methylpiperazin-1-yl)pyridin-3-yl}-18,18-dimethyl-16-oxa-6,10,22,28-tetraazapentacyclo[18.5.2.1\hat{ }{2,6}0.1\hat{ }{10,14}0.\hat{ }{23,27}]nonacosa-1 (26),2,20,23(27),24-pentaene-9,15-dione (70 mg, 0.10 mmol, 1.0 equiv) and (2S)-3-methyl-2-{methyl[3-(prop-2-enoyl)-1-oxa-3,8-diazaspiro[4.5]decan-8-yl]carbonylamino}butanoic acid (40 mg, 0.12 mmol, 1.2 equiv) in DMF(2 mL) was added HATU(44 mg, 0.12 mmol, 1.2 equiv) and DIEA (187 mg, 1.44 mmol, 15.0 equiv) at 0° C. The reaction mixture was stirred at 0° C. for 1 h. The solution was purified by chromatography to afford (2S)—N-[(8S,14S,20M)-22-ethyl-21-{2-[(1S)-1-methoxyethyl]-5-(4-methylpiperazin-1-yl)pyridin-3-yl}-18,18-dimethyl-9,15-dioxo-16-oxa-6,10,22,28-tetraazapentacyclo[18.5.2.1^{2,6} 0.1^{10,14}0.0^{23,27}]nonacosa-1(26),2,20,23(27),24-pentaen-8-yl]-3-methyl-2-{methyl[3-(prop-2-enoyl)-1-oxa-3,8-diazaspiro[4.5]decane-8-carbonyl]amino}butanamide (20.5 mg, 18% yield) as a white solid. ESI-MS m/z=1062.5[M+H]⁺, Calculated MW: 1061.64. ¹H NMR (400 MHz, CD₃OD) δ 8.43 (d, J=2.8 Hz, 1H), 7.58-7.49 (m, 2H), 7.45-7.35 (m, 2H), 6.65-6.41 (in, 1H), 6.38-6.32 (in, 1H), 6.29 (s, 1H), 5.91 (dd, J=8.8, 2.6 Hz, 1H), 5.84-5.78 (m, 1H), 5.21 (dd, J=7.6, 4.0 Hz, 1H), 5.12 (q, J=6.0 Hz, 1H), 4.50 (d, J=13.2 Hz, 1H), 4.18-3.02 (in, 3H), 3.00-3.81 (in, 4H), 3.76-3.70 (in, 1H), 3.59 (s, 1H), 3.52-3.42 (in, 2H), 3.42-3.34 (m, 6H), 3.28-3.20 (m, 2H), 3.14-3.07 (m, 1H), 3.06-2.98 (m, 3H), 2.90-2.74 (m, 7H), 2.70-2.62 (in, 4H), 2.62-2.56 (in, 1H), 2.44-2.29 (m, 6H), 2.26-2.17 (m, 1H), 2.16-2.09 (m, 1H), 2.00-1.90 (m, 1H), 1.89-1.78 (m, 3H), 1.76-1.64 (m, 3H), 1.44 (d, J=6.4 Hz, 3H), 1.21 (t, J=7.2 Hz, 2H), 0.95 (d, J=6.4 Hz, 3H), 0.92-0.82 (m, 6H), 0.71 (s, 3H).

### Synthesis of (2S)—N-[(6S,8S,14S,20P)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1^{2,6}.1^{10,14}0.0^{23,27}]nonacosa-1(26),20,23(27),24-tetraen-8-yl]-2-({3-[(2E)-4-fluorobut-2-enoyl]-1-oxa-3,8-diazaspiro[4.5]decane-8-carbonyl}(methyl)amino)-3-methylbutanamide

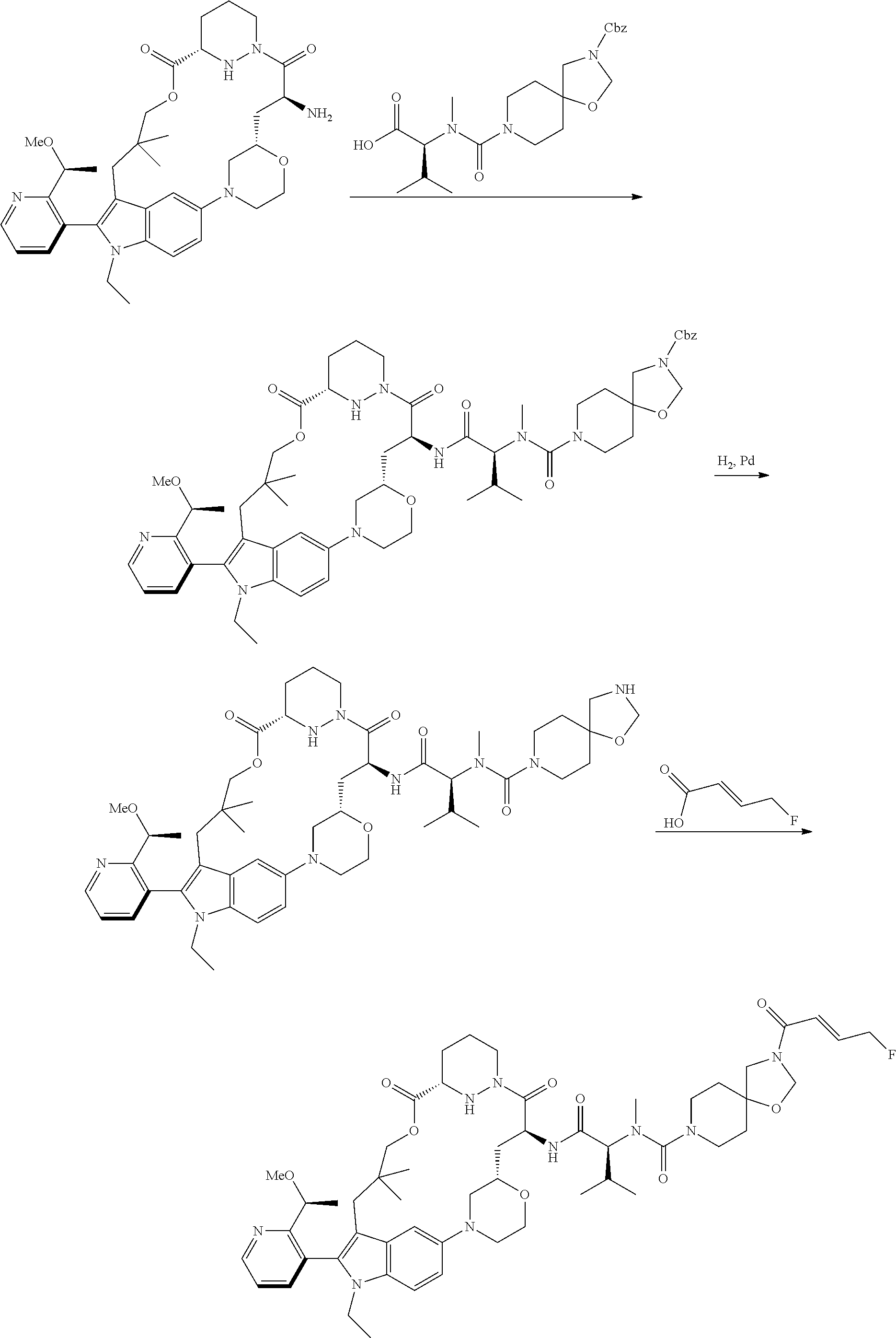

Step 1. To a solution of (2S)-2-[(3-{3-[(formyloxy)methyl]phenyl}-1-oxa-3,8-diazaspiro[4.5]decan-8-yl)carbonyl(methyl)amino]-3-methylbutanoic acid (308 mg, 0.71 mmol, 1.5 eq) and (6S,8S,14S)-8-amino-22-ethyl-21-{2-[(1S)-1-methoxyethyl]pyridin-3-yl}-18,18-dimethyl-5,16-dioxa-2,10,22,28-tetraazapentacyclo[18.5.2.1\hat{ }{2,6}0.1\hat{ }{10,14}0.0\hat{ }{23,27}]nonacosa-1(26),20,23(27),24-tetraene-9,15-dione (300 mg, 0.47 mmol, 1 eq) in DMF (3 mL) was added DIEA (184 mg, 1.4 mmol, 3 eq) and HATU (216 mg, 0.57 mmol, 1.2 eq) at 0° C. The mixture was stirred at 0° C. for 0.5 h. The reaction mixture was quenched with H₂O (30 mL), extracted with EtOAc (20 mL×3), and the combined organic layers were washed with water (20 mL), brine (20 mL), dried over Na₂SO₄. The mixture was filtered and concentrated under reduced pressure. The resulting residue was purified by chromatography to afford [3-(8-{[(1S)-1-{[(6S,8S,14S)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1\hat{ }{2,6}0.1\hat{ }{10,14}0.0\hat{ }{23,27}]nonacosa-1(26),20,23(27),24-tetraen-8-yl]carbamoyl}-2-methylpropyl](methyl)carbamoyl}-1-oxa-3,8-diazaspiro[4.5]decan-3-yl)phenyl]methyl formate (300 mg, 60% yield) as a white solid. ESI-MS m/z: 1048.5 [M+H]⁺, Calculated MW: 1047.6

Step 2. To a solution of [3-(8-{[(1S)-1-{[(6S,8S,14S)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1\hat{ }{2,6}0.1\hat{ }{10,14}0.0\hat{ }{23,27}]nonacosa-1(26),20,23(27),24-tetraen-8-yl]carbamoyl}-2-methylpropyl](methyl)carbamoyl}-1-oxa-3,8-diazaspiro[4.5]decan-3-yl)phenyl]methyl formate (300 mg, 0.29 mmol, 1 eq) in i-PrOH (10 mL) was added 20% Pd(OH)₂/C (30 mg, 60% water). The mixture was stirred at 20° C. for 20 min under H₂ (15 psi) atmosphere. The mixture was filtered and the filtrate was concentrated under reduced pressure to afford (2S)—N-[(6S,8S,14S)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1\hat{ }{2,6}0.1\hat{ }{10,14}0.0\hat{ }{23,27}]nonacosa-1 (26),20,23(27),24-tetraen-8-yl]-3-methyl-2-[methyl({1-oxa-3,8-diazaspiro[4.5]decan-8-yl}carbonyl)amino]butanamide (200 mg, 61% yield) as brown oil. ESI-MS m/z: 914.4 [M+H]⁺, Calculated MW: 913.5 Step 3. To a solution of (2S)—N-[(6S,8S,14S)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1\hat{ }{2,6}0.1\hat{ }{10,14}0.0\hat{ }{23,27}]nonacosa-1(26),20,23(27),24-tetraen-8-yl]-3-methyl-2-[methyl({1-oxa-3,8-diazaspiro[4.5]decan-8-yl}carbonyl)amino]butanamide (200 mg, 0.22 mmol, 1 eq), (2E)-4-fluorobut-2-enoic acid (23 mg, 0.22 mmol, 1 eq) and TEA (111 mg, 0.11 mmol, 5 eq) in DMF (3 mL) was added T3P (278 mg, 0.44 mmol, 2 eq, 50% EtOAc) at 0° C. The reaction mixture was stirred at 0° C. for 0.5 h. The reaction mixture was then quenched with water (20 mL) and the resulting mixture was extracted with EtOAc (15 mL×4). The combined organic phases were washed with brine (10 mL×4), dried over anhydrous sodium sulfate, filtered, and concentrated under reduced pressure. The resulting residue was purified by chromatography to afford (2S)—N-[(6S,8S,14S,20P)-22-ethyl-21-{2-[(1 S)-1-methoxyethyl]pyridin-3-yl}-18,18-dimethyl-9,15-dioxo-5,16-dioxa-2,10,22,28-tetraazapentacyclo[18.5.2.1^{2,6} 0.1^{10,14}0.0^{23,27}]nonacosa-1(26),20,23(27),24-tetraen-8-yl]-2-({3-[(2E)-4-fluorobut-2-enoyl]-1-oxa-3,8-diazaspiro[4.5]decane-8-carbonyl}(methyl)amino)-3-methylbutanamide (56.7 mg, 26% yield) as a white solid. ESI-MS m/z: 1000.6 [M+H]⁺, Calculated MW: 999.6. ¹H NMR (400 MHz, CD₃OD) δ 8.74 (dd, J=4.8, 1.6 Hz, 1H), 8.15 (d, J=6.0 Hz, 1H), 7.89 (dd, J=8.0, 1.6 Hz, 1H), 7.54 (dd, J=8.0, 4.8 Hz, 1H), 7.41 (d, J=9.2 Hz, 1H), 7.13-7.09 (m, 2H), 7.02-6.88 (m, 1H), 6.51-6.26 (m, 1H), 5.73-5.60 (m, 1H), 5.29-5.01 (m, 4H), 4.49 (d, J=12.8 Hz, 1H), 4.30-4.21 (m, 1H), 4.17-4.11 (m, 1H), 4.02-3.78 (m, 6H), 3.72-3.67 (m, 2H), 3.64-3.56 (m, 2H), 3.54-3.45 (m, 2H), 3.44-3.36 (m, 2H), 3.31-3.24 (m, 2H), 3.19 (s, 3H), 3.00-2.91 (m, 1H), 2.90-2.74 (m, 5H), 2.71-2.54 (m, 2H), 2.29-2.21 (m, 1H), 2.17-2.05 (m, 2H), 1.98-1.85 (m, 4H), 1.77-1.72 (m, 3H), 1.69-1.60 (m, 1H), 1.46 (d, J=6.0 Hz, 3H), 1.07 (t, J=6.4 Hz, 3H), 0.96 (d, J=6.4 Hz, 3H), 0.91 (d, J=7.6 Hz, 3H), 0.83-0.58 (m, 6H).

### Synthesis of (2S)—N-[(6S,8S,14S,20M)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1^{2,6}0.1^{10,14}0.0^{23,27}]nonacosa-1(26),20,23(27),24-tetraen-8-yl]-3-methyl-2-{methyl[7-(prop-2-enoyl)-5-oxa-2,7-diazaspiro[3.4]octane-2-carbonyl]amino}butanamide

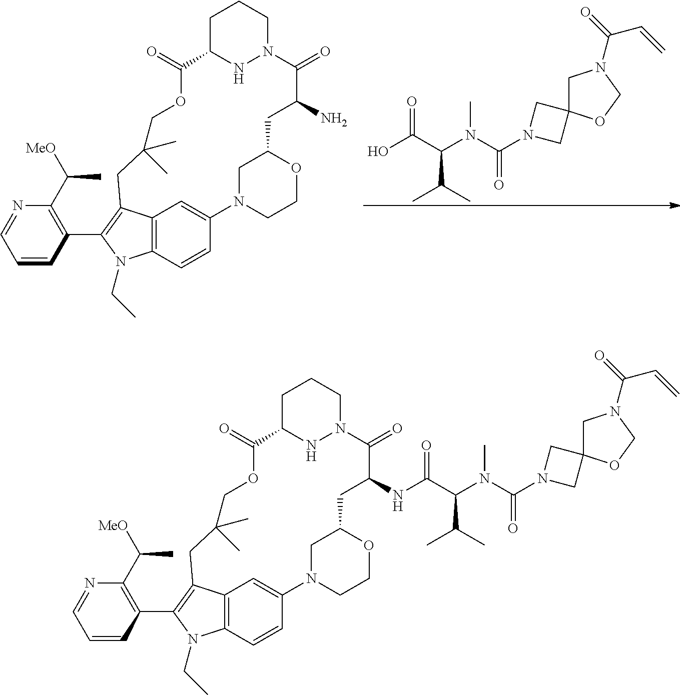

To a solution of the (6S,8S,14S)-8-amino-22-ethyl-21-{2-[(1S)-1-methoxyethyl]pyridin-3-yl}-18,18-dimethyl-5,16-dioxa-2,10,22,28-tetraazapentacyclo[18.5.2.1\hat{ }{2,6}0.1\hat{ }{10,14}0.0\hat{ }{23,27}]nonacosa-1(26),20,23(27),24-tetraene-9,15-dione (250 mg, 0.40 mmol, 1.0 equiv) and (2S)-3-methyl-2-{methyl[7-(prop-2-enoyl)-5-oxa-2,7-diazaspiro[3.4]octan-2-yl]carbonylamino}butanoic acid (200 mg, 0.60 mmol, 1.5 equiv) in DMF(4 mL) was added HATU(180 mg, 0.47 mmol, 1.2 equiv) and DIEA (766 mg, 5.2 mmol, 15 equiv) dropwise at 0° C. The reaction mixture was stirred at 0° C. for 1 h. The solution was purified by chromatography to afford (2S)—N-[(6S,8S,14S,20M)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1^{2,6}0.1^{10,14}0.0^{23,27}]nonacosa-1(26),20,23(27),24-tetraen-8-yl]-3-methyl-2-{methyl[7-(prop-2-enoyl)-5-oxa-2,7-diazaspiro[3.4]octane-2-carbonyl]amino}butanamide (124.3 mg, yield: 33%) as a white solid. ESI-MS m/z=940.5[M+H]⁺; Calculated MW: 939.52 ¹H NMR (400 MHz, CD₃OD) δ 8.71 (dd, J=4.8, 1.6 Hz, 1H), 7.86 (dd, J=7.6, 1.6 Hz, 1H), 7.51 (dd, J=7.6, 4.8 Hz, 1H), 7.39 (d, J=8.8 Hz, 1H), 7.16-7.05 (m, 2H), 6.61-6.27 (m, 2H), 5.86-5.76 (m, 1H), 5.67-5.58 (m, 1H), 5.18 (s, 1H), 5.09 (s, 1H), 4.46 (d, J=11.6 Hz, 1H), 4.29-4.19 (m, 3H), 4.18-4.10 (m, 3H), 4.07 (d, J=9.6 Hz, 1H), 3.99-3.90 (m, 3H), 3.89-3.82 (m, 1H), 3.82-3.78 (m, 2H), 3.77-3.66 (m, 2H), 3.54 (d, J=11.6 Hz, 1H), 3.16 (s, 3H), 2.94 (t, J=10.8 Hz, 1H), 2.85-2.74 (m, 5H), 2.71-2.64 (m, 1H), 2.63-2.49 (m, 1H), 2.26-2.16 (m, 1H), 2.15-2.05 (m, 2H), 1.92 (d, J=14.8 Hz, 2H), 1.81-1.69 (m, 1H), 1.68-1.56 (m, 1H), 1.44 (d, J=6.4 Hz, 3H), 1.33 (d, J=6.4 Hz, 2H), 1.04 (t, J=6.8 Hz, 3H), 0.95 (d, J=6.4 Hz, 3H), 0.88 (d, J=6.4 Hz, 3H), 0.76 (s, 3H), 0.68 (s, 3H).

### Synthesis of (2S)—N-[(6S,8S,14S,20M)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1^{2,6}0.1^{10,14}0.0^{23,27}]nonacosa-1(26),20,23(27),24-tetraen-8-yl]-3-methyl-2-{methyl[(5S)-3-(prop-2-enoyl)-1-oxa-3,7-diazaspiro[4.4]nonane-7-carbonyl]amino}butanamide

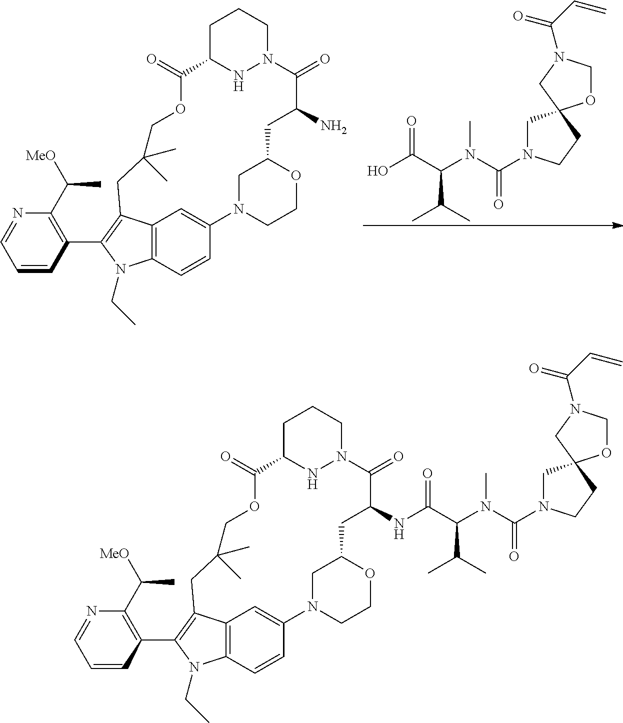

To a solution of the (6S,8S,14S)-8-amino-22-ethyl-21-{2-[(1S)-1-methoxyethyl]pyridin-3-yl}-18,18-dimethyl-5,16-dioxa-2,10,22,28-tetraazapentacyclo[18.5.2.1\hat{ }{2,6}0.1\hat{ }{10,14}0.0\hat{ }{23,27}]nonacosa-1(26),20,23(27),24-tetraene-9,15-dione (160 mg, 0.25 mmol, 1.0 equiv) and (2S)-3-methyl-2-{methyl[(5S)-3-(prop-2-enoyl)-1-oxa-3,7-diazaspiro[4.4]nonan-7-yl]carbonylamino}butanoic acid (137 mg, 0.4 mmol, 1.6 equiv) in DMF(4 mL) was added HATU (115 mg, 0.3 mmol, 1.2 equiv) and DIEA (490 mg, 3.7 mmol, 15.0 equiv) dropwise at 0° C. The reaction mixture was stirred at 0° C. for 1 h. The solution was purified by chromatography to afford (2S)—N-[(6S,8S,14S,20M)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1^{2, 6}0.1^{10,14}0.0^{23,27}]nonacosa-1(26),20,23(27),24-tetraen-8-yl]-3-methyl-2-{methyl[(5S)-3-(prop-2-enoyl)-1-oxa-3,7-diazaspiro[4.4]nonane-7-carbonyl]amino}butanamide (65.5 mg, yield: 27%) as white solid. ESI-MS m/z=954.5[M+H]⁺; Calculated MW: 953.54. ¹H NMR (400 MHz, CD30D δ 8.71 (dd, J=4.8, 1.6 Hz, 1H), 7.86 (dd, J=7.6, 1.6 Hz, 1H), 7.51 (dd, J=7.6, 4.8 Hz, 1H), 7.39 (d, J=8.8 Hz, 1H), 7.16-7.02 (m, 2H), 6.61-6.36 (m, 1H), 6.36-6.27 (m, 1H), 5.83-5.76 (m, 1H), 5.64 (d, J=7.6 Hz, 1H), 5.21 (dd, J=11.2, 4.0 Hz, 1H), 5.11 (q, J=6.0 Hz, 1H), 4.47 (d, J=12.0 Hz, 1H), 4.25-4.18 (m, 1H), 4.17-4.12 (m, 1H), 4.04 (d, J=11.2 Hz, 1H), 3.97-3.64 (m, 11H), 3.55 (d, J=11.6 Hz, 1H), 3.50-3.40 (m, 2H), 3.26 (s, 1H), 3.15 (s, 3H), 2.98-2.75 (m, 6H), 2.68-2.49 (m, 2H), 2.25-2.15 (m, 2H), 2.15-2.01 (m, 3H), 1.92 (d, J=14.8 Hz, 2H), 1.81-1.59 (m, 2H), 1.44 (d, J=6.0 Hz, 3H), 1.05 (t, J=6.4 Hz, 3H), 1.00-0.85 (m, 6H), 0.80-0.60 (m, 6H).

### Synthesis of Synthesis of (2S)—N-[(6S,8S,14S,20M)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1^{2,6}0.1^{10,14}0.0^{23,27}]nonacosa-1(26),20,23(27),24-tetraen-8-yl]-3-methyl-2-{methyl[(5R)-3-(prop-2-enoyl)-1-oxa-3,7-diazaspiro[4.4]nonane-7-carbonyl]amino}butanamide

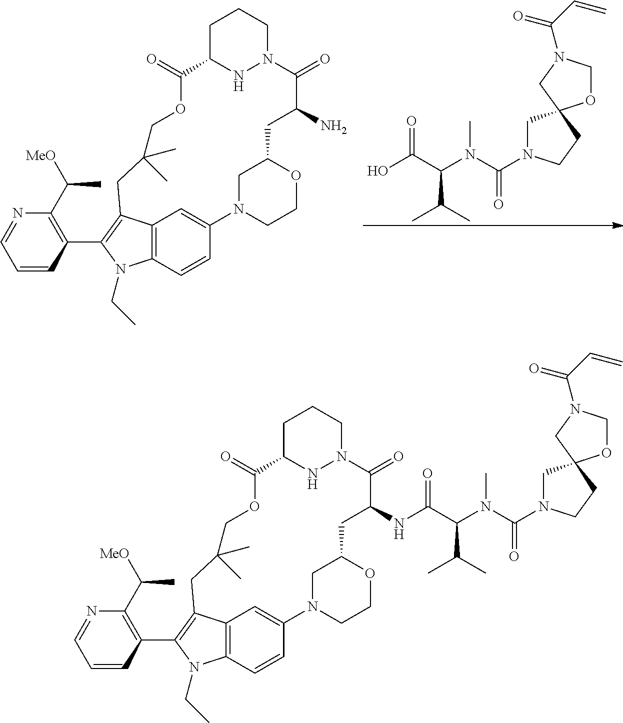

To a solution of (6S,8S,14S)-8-amino-22-ethyl-21-{2-[(1S)-1-methoxyethyl]pyridin-3-yl}-18,18-dimethyl-5,16-dioxa-2,10,22,28-tetraazapentacyclo[18.5.2.1\hat{ }{2,6}0.1\hat{ }{10,14}0.0\hat{ }{23,27}]nonacosa-1(26),20,23(27),24-tetraene-9,15-dione (160 mg, 0.25 mmol, 1.0 equiv) and (2S)-3-methyl-2-{methyl[3-(prop-2-enoyl)-1-oxa-3,7-diazaspiro[4.4]nonan-7-yl]carbonylamino}butanoic acid (103 mg, 0.30 mmol, 1.2 equiv) and DIPEA (653 mg, 5.1 mmol, 20 equiv) in DMF (1 mL) was added HATU (96 mg, 0.25 mmol, 1.0 equiv) at 0° C., then the mixture was stirred at 0-5° C. for 1 h. The mixture was diluted with EA (20 mL), then washed with water (20 mL*2) and brine (20 mL). The organic phase was collected, dried over Na₂SO₄, filtered and concentrated. The resulting residue was purified by chromatography to afford (2S)—N-[(6S,8S,14S)-22-ethyl-21-{2-[(1S)-1-methoxyethyl]pyridin-3-yl}-18,18-dimethyl-9,15-dioxo-5,16-dioxa-2,10,22,28-tetraazapentacyclo[18.5.2.1\hat{ }{2,6}0.1\hat{ }{10,14}0.0\hat{ }{23,27}]nonacosa-1 (26),20,23(27),24-tetraen-8-yl]-3-methyl-2-{methyl[(5R)-3-(prop-2-enoyl)-1-oxa-3,7-diazaspiro[4.4]nonan-7-yl]carbonylamino}butanamide (92 mg, 38% yield) as an off-white solid. ESI-MS m/z: 954.4 [M+H]⁺. Calculated MW: 953.54. 1 H NMR (400 MHz, MeOD) δ 8.72-8.70 (m, 1H), 7.85-7.82 (m, 1H), 7.51-7.51 (m, 1H), 7.38 (d, J=8.8 Hz, 1H), 7.15-7.01 (m, 2H), 6.59-6.41 (m, 1H), 6.35-6.27 (m, 1H), 5.85-5.73 (m, 1H), 5.64 (d, J=8.8 Hz, 1H), 5.19-5.10 (m, 1H), 5.10 (s, 1H), 4.46 (d, J=12.0 Hz, 1H), 4.23-4.11 (m, 2H), 3.92-3.82 (m, 7H), 3.76-3.63 (m, 4H), 3.50-3.49 (m, 4H), 3.26 (s, 1H), 3.15 (s, 3H), 2.95-2.74 (m, 1H), 2.88-2.73 (m, 5H), 2.65-2.54 (m, 2H), 2.29-2.18 (m, 1H), 2.23-2.02 (m, 4H), 1.92 (d, J=14.8 Hz, 2H), 1.73-1.62 (m, 2H), 1.43 (d, J=6.4 Hz, 3H), 1.10-1.02 (m, 3H), 0.94 (d, J=6.4 Hz, 3H), 0.88 (d, J=6.8 Hz, 3H), 0.60-0.50 (m, 6H).

TABLE E3
Exemplary Compounds Prepared by
Methods of the Present Invention

| | | LCMS |
|---|---|---|
| | Molecular | (ESI) |
| | weight | m/z |
| Ex# | (g/mol) | Found |
| EA1 | 938.2 | 938.7 |
| EA2 | 1080.38 | 1081.1 |
| EA3 | 1078.39 | 1078.7 |
| EA4 | 1071.378 | 1071.5 |
| EA5 | 1109.35 | 1109.8 |
| EA6 | 1087.396 | 1087.9 |
| EA7 | 1031.164 | 1031.9 |
| EA8 | 1055.379 | 1056.7 |
| EA9 | 1040.31 | 1040.9 |
| EA10 | 966.21 | 966.2 |
| EA11 | 1000.29 | 1000.7 |
| EA12 | 1006.182 | 1006.9 |
| EA13 | 991.219 | 991.9 |
| EA14 | 1087.396 | 1087.8 |
| EA15 | 1073.369 | 1073.4 |
| EA16 | 954.18 | 954.4 |
| EA17 | 1047.25 | 1047.6 |
| EA18 | 1014.161 | 1014.8 |
| EA19 | 959.202 | 959.1 |
| EA20 | 968.21 | 968.3 |
| EA21 | 940.156 | 940.5 |
| EA22 | 1066.306 | 1066.8 |
| EA23 | 990.095 | 990.5 |
| EA24 | 1010.198 | 1010.8 |
| EA25 | 1000.134 | 1000.7 |
| EA26 | 1006.182 | 1006.9 |
| EA27 | 950.195 | 950.7 |
| EA28 | 1033.35 | 1033.4 |
| EA29 | 1018.149 | 1018.9 |
| EA30 | 1100.315 | 1100.8 |
| EA31 | 1016.298 | 1016.8 |
| EA32 | 1069.285 | 1069.6 |
| EA33 | 1018.149 | 1018.7 |
| EA34 | 1064.334 | 1065.1 |
| EA35 | 1014.326 | 1014.4 |
| EA36 | 975.205 | 975.6 |
| EA37 | 950.151 | 950.5 |
| EA38 | 1009.307 | 1009.2 |
| EA39 | 1065.322 | 1065.6 |
| EA40 | 945.175 | 945.8 |
| EA41 | 982.237 | 982.8 |
| EA42 | 982.237 | 982.3 |
| EA43 | 1092.397 | 1092.6 |
| EA44 | 1066.359 | 1065.6 |
| EA45 | 931.148 | 931.7 |
| EA46 | 996.264 | 996.8 |
| EA47 | 978.249 | 978.2 |
| EA48 | 982.237 | 982.8 |
| EA49 | 977.192 | 977.5 |
| EA50 | 1083.389 | 1083.9 |
| EA51 | 1031.357 | 1031.5 |
| EA52 | 1062.371 | 1062.5 |
| EA53 | 1097.416 | 1098.0 |
| EA54 | 1019.346 | 1019.6 |
| EA55 | 977.265 | 977.5 |
| EA56 | 1019.346 | 1019.5 |
| EA57 | 972.245 | 972.7 |
| EA58 | 978.249 | 978.8 |
| EA59 | 1049.251 | 1049.5 |
| EA60 | 1111.443 | 1112.1 |
| EA61 | 949.211 | 949.5 |
| EA62 | 1097.416 | 1097.9 |
| EA63 | 978.249 | 978.8 |
| EA64 | 978.249 | 978.8 |
| EA65 | 963.238 | 963.45 |
| EA66 | 1006.182 | 1006.8 |
| EA67 | 991.292 | 991.6 |
| EA68 | 1000.227 | 1000.6 |
| EA69 | 1021.318 | 1021.5 |
| EA70 | 963.238 | 963.4 |
| EA71 | 952.211 | 952.6 |
| EA72 | 1023.309 | 1023.6 |
| EA73 | 995.28 | 995.5 |
| EA74 | 1017.33 | 1017.6 |
| EA75 | 1060.399 | 1060.6 |
| EA76 | 1041.3 | 1041.6 |
| EA77 | 1035.345 | 1035.5 |
| EA78 | 950.151 | 950.7 |
| EA79 | 1018.149 | 1018.7 |
| EA80 | 1023.334 | 1023.6 |
| EA81 | 1005.319 | 1005.5 |
| EA82 | 1023.334 | 1023.6 |
| EA83 | 1100.464 | 1100.6 |
| EA84 | 1009.307 | 1009.5 |
| EA85 | 1114.37 | 1114.6 |
| EA86 | 1032.316 | 1032.9 |
| EA87 | 1027.297 | 1027.5 |
| EA88 | 992.276 | 992.8 |
| EA89 | 1057.323 | 1057.8 |
| EA90 | 1008.275 | 1008.8 |
| EA91 | 945.175 | 945.9 |
| EA92 | 1037.34 | 1037.9 |
| EA93 | 978.249 | 978.8 |
| EA94 | 1006.182 | 1006.8 |
| EA95 | 1034.288 | 1034.9 |
| EA96 | 1059.29 | 1059.6 |
| EA97 | 1063.38 | 1063.9 |
| EA98 | 1012.263 | 1012.8 |
| EA99 | 1064.36 | 1064.8 |
| EA100 | 1050.33 | 1050.8 |
| EA101 | 1062.39 | 1062.9 |
| EA102 | 1064.36 | 1064.8 |
| EA103 | 1034.34 | 1034.8 |
| EA104 | 929.176 | 929.8 |
| EA105 | 952.19 | 952.3 |
| EA106 | 1002.219 | 1002.5 |
| EA107 | 1029.192 | 1029.6 |
| EA108 | 954.183 | 954.4 |
| EA109 | 1004.166 | 1004.8 |
| EA110 | 1062.342 | 1062.9 |
| EA111 | 1030.325 | 1030.9 |
| EA112 | 1104.452 | 1104.5 |
| EA113 | 888.083 | 888.4 |
| EA114 | 954.183 | 954.5 |
| EA115 | 1081.321 | 1081.9 |
| EA116 | 992.155 | 992.7 |
| EA117 | 1061.28 | 1062.7 |
| EA118 | 995.211 | 995.4 |
| EA119 | 979.26 | 979.9 |
| EA120 | 1080.333 | 1080.8 |
| EA121 | 952.211 | 952.4 |
| EA122 | 1065.39 | 1065.8 |
| EA123 | 938.115 | 938.8 |
| EA124 | 974.217 | 974.7 |
| EA125 | 1096.404 | 1097.0 |
| EA126 | 1090.425 | 1090.7 |
| EA127 | 995.211 | 995.8 |
| EA128 | 984.228 | 984.5 |
| EA129 | 1045.288 | 1045.6 |
| EA130 | 996.264 | 996.5 |
| EA131 | 1021.34 | 1021.3 |
| EA132 | 955.16 | 955.4 |
| EA133 | 986.272 | 986.8 |
| EA134 | 1085.284 | 1085.8 |
| EA135 | 935.155 | 935.5 |
| EA136 | 954.183 | 954.7 |
| EA137 | 1101.35 | 1101.9 |
| EA138 | 1048.315 | 1048.9 |
| EA139 | 994.227 | 994.7 |
| EA140 | 992.155 | 992.7 |
| EA141 | 1028.34 | 1029.5 |
| EA142 | 977.192 | 977.7 |
| EA143 | 1058.3 | 1058.7 |
| EA144 | 1047.37 | 1047.9 |
| EA145 | 1026.309 | 1026.5 |
| EA146 | 938.2 | 938.4 |
| EA147 | 1010.291 | 1010.5 |
| EA148 | 1060.34 | 1060.4 |
| EA149 | 934.123 | 934.7 |
| EA150 | 1094.41 | 1095.0 |
| EA151 | 976.25 | 976.5 |
| EA152 | 1047.37 | 1047.8 |
| EA153 | 938.2 | 938.4 |
| EA154 | 997.24 | 997.5 |
| EA155 | 1068.334 | 1068.9 |
| EA156 | 1063.278 | 1063.5 |
| EA157 | 1148.38 | 1148.5 |
| EA158 | 1116.36 | 1116.6 |
| EA159 | 992.276 | 992.5 |
| EA160 | 950.21 | 950.5 |
| EA161 | 1103.32 | 1104.0 |
| EA162 | 1047.37 | 1047.6 |
| EA163 | 1038.223 | 1038.8 |
| EA164 | 938.2 | 938.5 |
| EA165 | 1069.241 | 1069.6 |
| EA166 | 1019.2 | 1019.7 |
| EA167 | 1060.34 | 1060.4 |
| EA168 | 950.21 | 950.9 |
| EA169 | 931.192 | 931.5 |
| EA170 | 950.21 | 950.8 |
| EA171 | 955.27 | 955.5 |
| EA172 | 1103.395 | 1103.6 |
| EA173 | 1118.358 | 1118.4 |
| EA174 | 1118.358 | 1118.4 |
| EA175 | 955.27 | 955.5 |
| EA176 | 1082.27 | 1082.4 |
| EA177 | 964.222 | 964.4 |
| EA178 | 964.222 | 964.4 |
| EA179 | 1080.386 | 1080.4 |
| EA180 | 1064.387 | 1064.4 |
| EA181 | 1064.387 | 1064.4 |
| EA182 | 979.184 | 979.5 |
| EA183 | 1048.315 | 1048.8 |
| EA184 | 950.21 | 950.7 |
| EA185 | 986.272 | 986.9 |
| EA186 | 996.264 | 996.3 |
| EA187 | 1037.268 | 1037.7 |
| EA188 | 1041.231 | 1041.7 |
| EA189 | 995.28 | 995.9 |
| EA190 | 1037.316 | 1037.8 |
| EA191 | 975.205 | 975.8 |
| EA192 | 1019.346 | 1019.8 |
| EA193 | 978.249 | 978.7 |
| EA194 | 1005.319 | 1005.6 |
| EA195 | 977.265 | 977.6 |
| EA196 | 978.249 | 978.7 |
| EA197 | 991.292 | 991.4 |
| EA198 | 1018.289 | 1018.9 |
| EA199 | 981.253 | 981.5 |
| EA200 | 1036.28 | 1036.9 |
| EA201 | 1035.345 | 1035.5 |
| EA202 | 1004.262 | 1004.5 |
| EA203 | 981.253 | 981.5 |
| EA204 | 1119.414 | 1119.9 |
| EA205 | 980.265 | 980.5 |
| EA206 | 1021.318 | 1021.6 |
| EA207 | 1039.333 | 1039.5 |
| EA208 | 993.28 | 993.8 |
| EA209 | 1141.368 | 1141.8 |

### Example 13. Biological Assays of Compounds of Table E1

All but 10 Compounds of Table E1 herein exhibited an IC₅₀ of 1 μM or less in the H358 (K-Ras G12C) pERK potency assay described below. Ten compounds exceeded 1 μM (EA36, EA37, EA38, EA121, EA124, EA128, EA136, EA189, EA191, EA192). Compound EA130 had an IC₅₀ greater than 0.89 μM. Compounds of Table E1 herein exhibited an IC₅₀ of 3 μM or less in the MiaPaCa-2 (K-Ras G13C) pERK potency assay described below.

All but 5 compounds in Table E1 exhibited an IC₅₀ less than 1 μM in a cell viability assay described below (NCI-H358 (K-Ras G12C)). Five compounds exceeded 1 μM (EA38, EA39, EA128, EA191, EA192).

All compounds in Table E1 exhibited an IC₅₀ less than 3.5 μM in the Raf-Ras (FRET or MOA) binding assay described below re: K-Ras G12C. All but 3 compounds in Table E1 exhibited an IC₅₀ less than 1.5 μM in the Raf-Ras (FRET or MOA) binding assay described below re: K-Ras G13C. Three compounds exceeded 1.5 μM (EA169, EA171, EA175).

All compounds in Table E1 except EA168 and EA170 exhibited a cross-linking percent of greater than 0 under an incubation timeframe of 4 hours in the cross-linking assay described below with respect to K-Ras G12C or K-Ras G13C.

Potency Assay: pERK

The purpose of this assay was to measure the ability of test compounds to inhibit K-Ras in cells. Activated K-Ras induces increased phosphorylation of ERK at Threonine 202 and Tyrosine 204 (pERK). This procedure measures a decrease in cellular pERK in response to test compounds. The procedure described below in NCI-H358 cells is applicable to K-Ras G12C.

Note: This protocol may be executed substituting other cell lines to characterize inhibitors of other RAS variants, including, for example, AsPC-1 (K-Ras G12D), Capan-1 (K-Ras G12V), or NCI-H1355 (K-Ras G13C).

NCI-H358 cells were grown and maintained using media and procedures recommended by the ATCC. On the day prior to compound addition, cells were plated in 384-well cell culture plates (40 μl/well) and grown overnight in a 37° C., 5% CO₂ incubator. Test compounds were prepared in 10, 3-fold dilutions in DMSO, with a high concentration of 10 mM. On the day of assay, 40 nL of test compound was added to each well of cell culture plate using an Echo550 liquid handler (LabCyte®). Concentrations of test compound were tested in duplicate. After compound addition, cells were incubated 4 hours at 37° C., 5% CO₂. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μL lysis buffer, with shaking at 600 RPM at room temperature. Lysate (10 μL) was transferred to a 384-well Opti-plate (PerkinElmer) and 5 μL acceptor mix was added. After a 2-hour incubation in the dark, 5 μL donor mix was added, the plate was sealed, and incubated 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 μL tris buffered saline (TBS) and fixed for 15 minutes with 150 μL 4% formaldehyde in TBS. Fixed cells were washed 4 times for 5 minutes with TBS containing 0.1% Triton X-100 (TBST) and then blocked with 100 μL Odyssey blocking buffer (LI-COR) for 60 minutes at room temperature. Primary antibody (pERK, CST-4370, Cell Signaling Technology) was diluted 1:200 in blocking buffer, and 50 μL were added to each well and incubated overnight at 4° C. Cells were washed 4 times for 5 minutes with TBST. Secondary antibody (IR-800CW rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a Li-COR Odyssey CLx Imager. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

Regarding G13C, another pERK assay protocol is as follows.

Note: This protocol may be executed substituting other cell lines to characterize inhibitors of other RAS variants, including, for example, AsPC-1 (K-Ras G12D), Capan-1 (K-Ras G12V), or NCI-H358 (K-Ras G12C).

MIA PaCa-2 KRAS G13C A12 cells were grown and maintained using media and procedures recommended by the ATCC. On the day prior to compound addition, cells were plated in 384-well cell culture plates (8,000 cells/40 μl/well) and grown overnight in a 37° C., 5% CO₂ incubator. Test compounds were prepared in 10, 3-fold dilutions in DMSO, with a high concentration of 10, 1 or 0.1 mM. On the day of assay, 40 nL of test compound were added to each well of cell culture plate using an Echo550 liquid handler (LabCyte®). Concentrations of test compound were tested in duplicate. After compound addition, cells were incubated 4 hours at 37° C., 5% CO₂. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μL lysis buffer, with shaking at 600 RPM at room temperature. Lysate (10 μL) was transferred to a 384-well Opti-plate (PerkinElmer) and 5 μL acceptor mix was added. After a 2-hour incubation in the dark, 5 μL donor mix was added, the plate was sealed, and incubated 2 hours at room temperature. Signal was read on an Envision plate reader (PerkinElmer) using standard AlphaLISA settings. Analysis of raw data was carried out in Genedata Screener and Prism (GraphPad). Data were normalized by the following calculation: ((sample signal−average low control)/(average DMSO−average low control))*100. Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 μL tris buffered saline (TBS) and fixed for 15 minutes with 150 μL 4% formaldehyde in TBS. Fixed cells were washed 4 times for 5 minutes with TBS containing 0.1% Triton X-100 (TBST) and then blocked with 100 μL Odyssey blocking buffer (LI-COR) for 60 minutes at room temperature. Primary antibody (pERK, CST-4370, Cell Signaling Technology) was diluted 1:200 in blocking buffer, and 50 μL were added to each well and incubated overnight at 4° C. Cells were washed 4 times for 5 minutes with TBST. Secondary antibody (IR-800CW rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a Li-COR Odyssey CLx Imager. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

### Determination of Cell Viability in RAS Mutant Cancer Cell Lines

### Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay was to determine the effects of test compounds on the proliferation of three human cancer cell lines (NCI-H358 (K-Ras G12C), AsPC-1 (K-Ras G12D), and Capan-1 (K-Ras G12V)) over a 5-day treatment period by quantifying the amount of ATP present at endpoint using the CellTiter-Glo® 2.0 Reagent (Promega).

Cells were seeded at 250 cells/well in 40 μL of growth medium in 384-well assay plates and incubated overnight in a humidified atmosphere of 5% CO₂ at 37° C. On the day of the assay, 10 mM stock solutions of test compounds were first diluted into 3 mM solutions with 100% DMSO. Well-mixed compound solutions (15 μL) were transferred to the next wells containing 30 μL of 100% DMSO, and repeated until a 9-concentration 3-fold serial dilution was made (starting assay concentration of 10 μM). Test compounds (132.5 nL) were directly dispensed into the assay plates containing cells. The plates were shaken for 15 seconds at 300 rpm, centrifuged, and incubated in a humidified atmosphere of 5% CO₂ at 37° C. for 5 days. On day 5, assay plates and their contents were equilibrated to room temperature for approximately 30 minutes. CellTiter-Glo® 2.0 Reagent (25 μL) was added, and plate contents were mixed for 2 minutes on an orbital shaker before incubation at room temperature for 10 minutes. Luminescence was measured using the PerkinElmer Enspire. Data were normalized by the following: (Sample signal/Avg. DMSO)*100. The data were fit using a four-parameter logistic fit.

Another CTG assay protocol employed with respect to MIA PaCa-2 KRAS G13C A12 (K-Ras G13C, in particular, is as follows, Note: other RAS isoforms may be employed (e.g., NCI-H358 (K-Ras G12C), AsPC-1 (K-Ras G12D), and Capan-1 (K-Ras G12V)), though the number of cells to be seeded will vary based on cell line used).

The purpose of this cellular assay was to determine the effects of test compounds on the proliferation of human cancer cell lines over a 5-day treatment period by quantifying the amount of ATP present at endpoint using the CellTiter-Glo® 2.0 Reagent (Promega).

Cells were seeded at 250 cells/well in 40 μL of growth medium in 384-well assay plates and incubated overnight in a humidified atmosphere of 5% CO₂ at 37° C. Test compounds were prepared in 9 point, 3-fold dilutions in DMSO, with a high concentration of 10, 1 or 0.1 mM. On the day of the assay, test compounds (40 nL) were directly dispensed into the assay plates containing cells. The plates were shaken for 15 seconds at 300 rpm, centrifuged, and incubated in a humidified atmosphere of 5% CO₂ at 37° C. for 5 days. On day 5, assay plates and their contents were equilibrated to room temperature for approximately 30 minutes. CellTiter-Glo® 2.0 Reagent (25 μL) was added, and plate contents were mixed for 2 minutes on an orbital shaker before incubation at room temperature for 10 minutes. Luminescence was measured using the PerkinElmer Enspire. Data were normalized by the following: (Sample signal/Avg. DMSO)*100. The data were fit using a four-parameter logistic fit.

Disruption of B-Raf Ras-Binding Domain (BRAF^{RBD}) Interaction with K-Ras by Compounds of the Invention (Also Called a FRET Assay or an MOA Assay)

Note—The following protocol describes a procedure for monitoring disruption of K-Ras G12C (GMP-PNP) binding to BRAF^{RBD} by a compound of the invention. This protocol may also be executed substituting other Ras proteins or nucleotides.

The purpose of this biochemical assay was to measure the ability of test compounds to facilitate ternary complex formation between a nucleotide-loaded K-Ras isoform and Cyclophilin A; the resulting ternary complex disrupts binding to a BRAF^{RBD} construct, inhibiting K-Ras signaling through a RAF effector. Data was reported as IC₅₀ values.

In assay buffer containing 25 mM HEPES pH 7.3, 0.002% Tween20, 0.1% BSA, 100 mM NaCl and 5 mM MgCl₂, tagless Cyclophilin A, His 6-K-Ras-GMPPNP, and GST-BRAF^{RBD} were combined in a 384-well assay plate at final concentrations of 25 μM, 12.5 nM and 50 nM, respectively. Compound was present in plate wells as a 10-point 3-fold dilution series starting at a final concentration of 30 μM. After incubation at 25° C. for 3 hours, a mixture of Anti-His Eu-W1024 and anti-GST allophycocyanin was then added to assay sample wells at final concentrations of 10 nM and 50 nM, respectively, and the reaction incubated for an additional 1.5 hours. TR-FRET signal was read on a microplate reader (Ex 320 nm, Em 665/615 nm). Compounds that facilitate disruption of a K-Ras:RAF complex were identified as those eliciting a decrease in the TR-FRET ratio relative to DMSO control wells.

Cross-Linking of Ras Proteins with Compounds of the Invention to Form Conjugates

The following cross-linking assay describes a method of determining covalent adduct formation by a compound of the present invention with a Ras protein.

(Note—the following protocol describes a procedure for monitoring cross-linking of K-Ras G12C (GMP-PNP) to a compound of the invention. This protocol may also be executed substituting other Ras proteins or nucleotides).

The purpose of this biochemical assay was to measure the ability of test compounds to covalently label nucleotide-loaded K-Ras isoforms. In assay buffer containing 12.5 mM HEPES pH 7.4, 75 mM NaCl, 1 mM MgCl₂, 1 mM BME, 5 μM Cyclophilin A and 2 μM test compound, a 5 μM stock of GMP-PNP-loaded K-Ras (1-169) G12C was diluted 10-fold to yield a final concentration of 0.5 μM; with final sample volume being 100 μL.

The sample was incubated at 25° C. for a time period of up to 24 hours prior to quenching by the addition of 10 μL of 5% Formic Acid. Quenched samples were centrifuged at 15000 rpm for 15 minutes in a benchtop centrifuge before injecting a 10 μL aliquot onto a reverse phase C4 column and eluting into a mass spectrometer with an increasing acetonitrile gradient in the mobile phase. Analysis of raw data was carried out using Waters MassLynx MS software, with % bound calculated from the deconvoluted protein peaks for labeled and unlabeled K-Ras.

### In Vitro Cell Proliferation Panels

Potency for inhibition of cell growth may be assessed at CrownBio using standard methods. Briefly, cell lines are cultured in appropriate medium, and then plated in 3D methylcellulose. Inhibition of cell growth is determined by CellTiter-Glo® after 5 days of culture with increasing concentrations of compounds. Compound potency is reported as the 50% inhibition concentration (absolute IC₅₀).

The assay took place over 7 days. On day 1, cells in 2D culture are harvested during logarithmic growth and suspended in culture medium at 1×105 cells/ml. Higher or lower cell densities are used for some cell lines based on prior optimization. 3.5 ml of cell suspension is mixed with 6.5% growth medium with 1% methylcellulose, resulting in a cell suspension in 0.65% methylcellulose. 90 μl of this suspension is distributed in the wells of 2 96-well plates. One plate is used for day 0 reading and 1 plate is used for the end-point experiment. Plates are incubated overnight at 37C with 5% CO₂. On day 2, one plate (for t0 reading) is removed and 10 μl growth medium plus 100 μl CellTiter-Glo® Reagent is added to each well. After mixing and a 10 minute incubation, luminescence is recorded on an EnVision Multi-Label Reader (Perkin Elmer). Compounds in DMSO are diluted in growth medium such that the final, maximum concentration of compound is 10 μM, and serial 4-fold dilutions are performed to generate a 9-point concentration series. 10 μl of compound solution at 10 times final concentration is added to wells of the second plate. Plate is then incubated for 120 hours at 37C and 5% CO₂. On day 7 the plates are removed, 100 μl CellTiter-Glo® Reagent is added to each well, and after mixing and a 10 minute incubation, luminescence is recorded on an EnVision Multi-Label Reader (Perkin Elmer). Data is exported to GeneData Screener and modeled with a sigmoidal concentration response model in order to determine the IC₅₀ for compound response.

Not all cell lines with a given RAS mutation may be equally sensitive to a RAS inhibitor targeting that mutation, due to differential expression of efflux transporters, varying dependencies on RAS pathway activation for growth, or other reasons. This has been exemplified by the cell line KYSE-410 which, despite 8 having a KRAS G12C mutation, is insensitive to the KRAS G12C (OFF) inhibitor MRTX-849 (Hallin et al., Cancer Discovery 10:54-71 (2020)), and the cell line SW1573, which is insensitive to the KRAS G12C (OFF) inhibitor AMG510 (Canon et al., Nature 575:217-223 (2019)).

### Example 14. Chemical Synthesis of Compounds of Tables F1-F6

Definitions used in this example are defined above in Example 12.

### Synthesis of Intermediates

### Intermediate F1. Synthesis of 3-(5-bromo-1-ethyl-2-[2-[(1S)-1-methoxyethyl]pyridin-3-yl]indol-3-yl)-2,2-dimethylpropan-1-ol

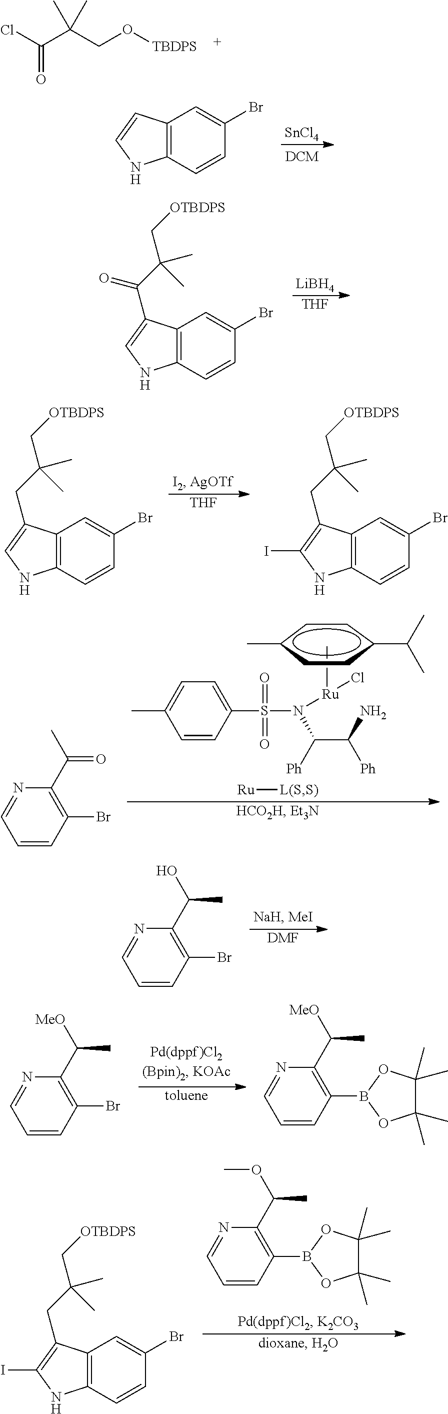

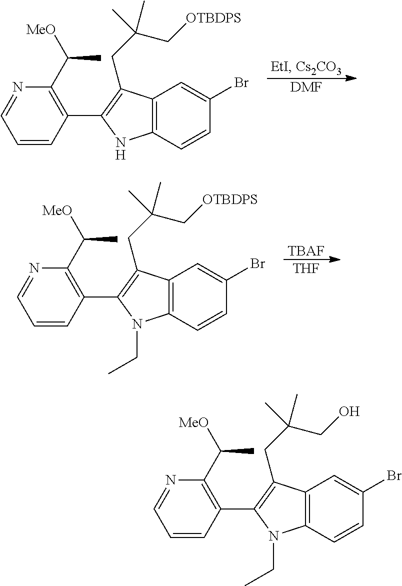

### Step 1: Synthesis of 1-(5-bromo-1H-indol-3-yl)-3-((tert-butyldiphenylsilyl)oxy)-2,2-dimethylpropan-1-one

To a mixture of 3-((tert-butyldiphenylsilyl)oxy)-2,2-dimethylpropanoyl chloride (65 g, 137 mmol, crude) in DCM (120 mL) at 0° C. under an atmosphere of N₂ was added 1M SnCl₄ in DCM (137 mL, 137 mmol) slowly. The mixture was stirred at 0° C. for 30 min, then a solution of 5-bromo-1H-indole (26.8 g, 137 mmol) in DCM (40 mL) was added dropwise. The mixture was stirred at 0° C. for 45 min, then diluted with EtOAc (300 mL), washed with brine (4×100 mL), dried over Na₂SO₄ and filtered. The filtrate was concentrated under reduced pressure and the residue was purified by silica gel silica gel column chromatography to the product (55 g, 75% yield). LCMS (ESI) m/z [M+Na] calcd for C₂₉H₃₂BrNO₂SiNa 556.1; found: 556.3.

### Step 2: Synthesis of 1-(5-bromo-1H-indol-3-yl)-3-((tert-butyldiphenylsilyl)oxy)-2,2-dimethylpropan-1-one

To a mixture of 1-(5-bromo-1H-indol-3-yl)-3-((tert-butyldiphenylsilyl)oxy)-2,2-dimethylpropan-1-one (50 g, 93.6 mmol) in THE (100 mL) at 0° C. under an atmosphere of N₂ was added LiBH₄ (6.1 g, 281 mmol). The mixture was heated to 60° C. and stirred for 20 h, then MeOH (10 mL) and EtOAc (100 mL) were added and the mixture washed with brine (50 mL), dried over Na₂SO₄, filtered and the filtrate concentrated under reduced pressure. The residue was diluted with DCM (50 mL), cooled to 10° C. and diludine (9.5 g, 37.4 mmol) and TsOH⋅H₂O (890 mg, 4.7 mmol) were added. The mixture was stirred at 10° C. for 2 h, filtered, the filtrate concentrated under reduced pressure and the residue was purified by silica gel column chromatography to give the product (41 g, 84% yield). LCMS (ESI) m/z [M+H] calcd for C₂₉H₃₄BrNOSi: 519.2; found: 520.1

### Step 3: Synthesis of 5-bromo-3-(3-((tert-butyldiphenylsilyl)oxy)-2,2-dimethylpropyl)-2-iodo-1H-indole

To a mixture of 1-(5-bromo-1H-indol-3-yl)-3-((tert-butyldiphenylsilyl)oxy)-2,2-dimethylpropan-1-one (1.5 g, 2.9 mmol) and 12 (731 mg, 2.9 mmol) in THE (15 mL) at room temperature was added AgOTf (888 mg, 3.5 mmol). The mixture was stirred at room temperature for 2 h, then diluted with EtOAc (200 mL) and washed with sat. aq. Na₂S₂O₃ (100 mL), dried over anhydrous Na₂SO₄ and filtered. The filtrate was concentrated under reduced pressure and the residue was purified by silica gel column chromatography to afford the product (900 mg, 72% yield) as a solid.

### Step 4: Synthesis of (1 S)-1-(3-bromopyridin-2-yl)ethanol

To a stirred mixture of HCO₂H (66.3 g, 1.44 mol) in Et₃N (728 g, 7.2 mol) at 0° C. under an atmosphere of Ar was added (4S,5S)-2-chloro-2-methyl-1-(4-methylbenzenesulfonyl)-4,5-diphenyl-1,3-diaza-2-ruthenacyclopentane cymene (3.9 g, 6.0 mmol) portion-wise. The mixture was heated to 40° C. and stirred for 15 min, then cooled to room temperature and 1-(3-bromopyridin-2-yl)ethanone (120 g, 600 mmol) added in portions. The mixture was heated to 40° C. and stirred for an additional 2 h, then the solvent was concentrated under reduced pressure. Brine (2 L) was added to the residue, the mixture was extracted with EtOAc (4×700 mL), dried over anhydrous Na₂SO₄ and filtered. The filtrate was concentrated under reduced pressure and the residue was purified by silica gel column chromatography to give the product (100 g, 74% yield) as an oil. LCMS (ESI) m/z [M+H] calcd for C₇H₈BrNO: 201.98; found: 201.9.

### Step 5: Synthesis of 3-bromo-2-[(1 S)-1-methoxyethyl]pyridine

To a stirred mixture of (1S)-1-(3-bromopyridin-2-yl)ethanol (100 g, 495 mmol) in DMF (1 L) at 0° C. was added NaH, 60% dispersion in oil (14.25 g, 594 mmol) in portions. The mixture was stirred at 0° C. for 1 h. Mel (140.5 g, 990 mmol) was added dropwise at 0° C. and the mixture was warmed to room temperature and stirred for 2 h. The mixture was cooled to 0° C. and sat. aq. NH₄Cl (5 L) was added. The mixture was extracted with EtOAc (3×1.5 L), dried over anhydrous Na₂SO₄ and filtered. The filtrate was concentrated under reduced pressure and the residue was purified by silica gel column chromatography to give the product (90 g, 75% yield) as an oil. LCMS (ESI) m/z [M+H] calcd for CH₁₀BrNO: 215.99; found: 215.9.

### Step 6: Synthesis of 2-[(1 S)-1-methoxyethyl]-3-(4,4,5,5-tetramethyl-1,3,2-dioxaborolan-2-yl)pyridine

To a stirred mixture of 3-bromo-2-[(1S)-1-methoxyethyl]pyridine (90 g, 417 mmol) in toluene (900 mL) at room temperature under an atmosphere of Ar was added bis(pinacolato)diboron (127 g, 500 mmol) and KOAc (81.8 g, 833 mmol) and Pd(dppf)Cl₂ (30.5 g, 41.7 mmol). The mixture was heated to 100° C. and stirred for 3 h. The filtrate was concentrated under reduced pressure and the residue was purified by Al₂O₃ column chromatography to give the product (100 g, 63% yield) as a semi-solid. LCMS (ESI) m/z [M+H] calcd for C₁₄H₂₂BNO₃: 264.17; found: 264.1.

### Step 7: Synthesis of 5-bromo-3-[3-[(tert-butyldiphenylsilyl)oxy]-2,2-dimethylpropyl]-2-[2-[(1 S)-1-methoxyethyl]pyridin-3-yl]-1H-indole

To a stirred mixture of 5-bromo-3-[3-[(tert-butyldiphenylsilyl)oxy]-2,2-dimethylpropyl]-2-iodo-1H-indole (140 g, 217 mmol) and 2-[(1 S)-1-methoxyethyl]-3-(4,4,5,5-tetramethyl-1,3,2-dioxaborolan-2-yl)pyridine (100 g, 380 mmol) in dioxane (1.4 L) at room temperature under an atmosphere of Ar was added K₂CO₃ (74.8 g, 541 mmol), Pd(dppf)Cl₂ (15.9 g, 21.7 mmol) and H₂O (280 mL) in portions. The mixture was heated to 85° C. and stirred for 4 h, then cooled, H₂O (5 L) added and the mixture extracted with EtOAc (3×2 L). The combined organic layers were washed with brine (2×1 L), dried over anhydrous Na₂SO₄ and filtered. The filtrate was concentrated under reduced pressure and the residue was purified by silica gel column chromatography to give the product (71 g, 45% yield) as a solid. LCMS (ESI) m/z [M+H] calcd for C₃₇H₄₃BrN₂O₂Si: 655.23; found: 655.1.

### Step 8: Synthesis of 5-bromo-3-[3-[(tert-butyldiphenylsilyl)oxy]-2,2-dimethylpropyl]-1-ethyl-2-[2-[(1 S)-1-methoxyethyl]pyridin-3-yl]indole

To a stirred mixture of 5-bromo-3-[3-[(tert-butyldiphenylsilyl)oxy]-2,2-dimethylpropyl]-2-[2-[(1 S)-1-methoxyethyl]pyridin-3-yl]-1H-indole (71 g, 108 mmol) in DMF (0.8 L) at 0° C. under an atmosphere of N₂ was added Cs₂CO₃ (70.6 g, 217 mmol) and Etl (33.8 g, 217 mmol) in portions. The mixture was warmed to room temperature and stirred for 16 h then H₂O (4 L) was added and the mixture extracted with EtOAc (3×1.5 L). The combined organic layers were washed with brine (2×1 L), dried over anhydrous Na₂SO₄ and filtered. The filtrate was concentrated under reduced pressure and the residue was purified by silica gel column chromatography to give the product (66 g, 80% yield) as an oil. LCMS (ESI) m/z [M+H] calcd for C₃₉H₄₇BrN₂O₂Si: 683.26; found: 683.3.

### Step 9: Synthesis of 3-(5-bromo-1-ethyl-2-[2-[(1 S)-1-methoxyethyl]pyridin-3-yl]indol-3-yl)-2,2-dimethylpropan-1-ol

To a stirred mixture of TBAF (172.6 g, 660 mmol) in THE (660 mL) at room temperature under an atmosphere of N₂ was added 5-bromo-3-[3-[(tert-butyldiphenylsilyl)oxy]-2,2-dimethylpropyl]-1-ethyl-2-[2-[(1 S)-1-methoxyethyl]pyridin-3-yl]indole (66 g, 97 mmol) in portions. The mixture was heated to 50° C. and stirred for 16 h, cooled, diluted with H₂O (5 L) and extracted with EtOAc (3×1.5 L). The combined organic layers were washed with brine (2×1 L), dried over anhydrous Na₂SO₄ and filtered. After filtration, the filtrate was concentrated under reduced pressure and the residue was purified by silica gel column chromatography to give the product (30 g, 62% yield) as a solid. LCMS (ESI) m/z [M+H] calcd for C₂₃H₂₉BrN₂O₂: 445.14; found: 445.1.

### Intermediate F2. Alternative Synthesis Through Fisher Indole Route

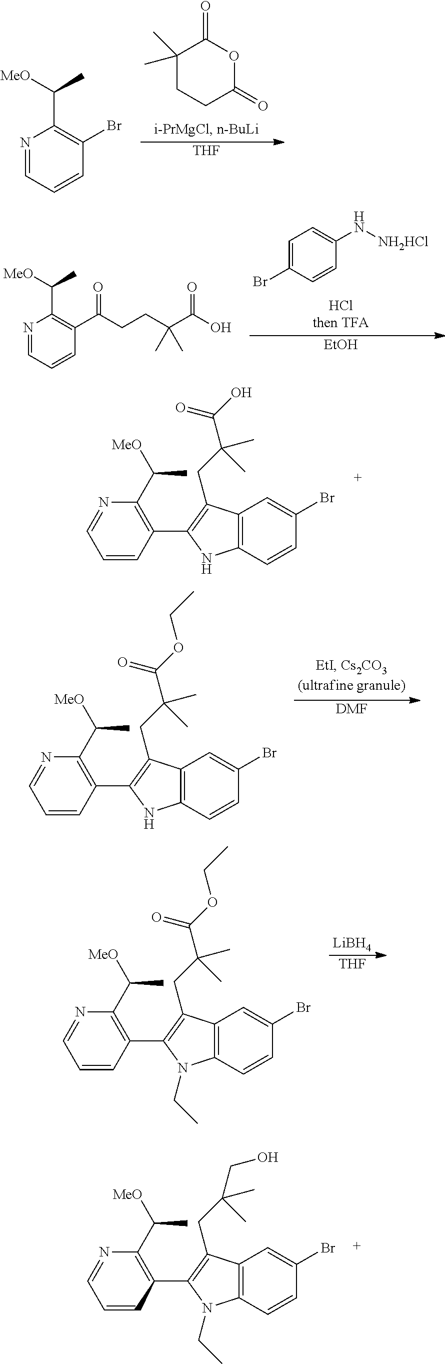

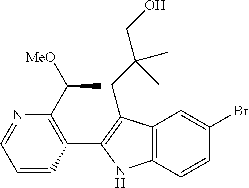

### Step 1: Synthesis of 5-[2-[(1 S)-1-methoxyethyl]pyridin-3-yl]-2,2-dimethyl-5-oxopentanoic acid

To a mixture of i-PrMgCl (2M in in THF, 0.5 L) at −10° C. under an atmosphere of N₂ was added n-BuLi, 2.5 M in hexane (333 mL, 833 mmol) dropwise over 15 min. The mixture was stirred for 30 min at −10° C. then 3-bromo-2-[(1S)-1-methoxyethyl]pyridine (180 g, 833 mmol) in THE (0.5 L) added dropwise over 30 min at −10° C. The resulting mixture was warmed to −5° C. and stirred for 1 h, then 3,3-dimethyloxane-2,6-dione (118 g, 833 mmol) in THE (1.2 L) was added dropwise over 30 min at −5° C. The mixture was warmed to 0° C. and stirred for 1.5 h, then quenched with the addition of pre-cooled 4M HCl in dioxane (0.6 L) at 0° C. to adjust pH ˜5. The mixture was diluted with H₂O (3 L) at 0° C. and extracted with EtOAc (3×2.5 L). The combined organic layers were dried over anhydrous Na₂SO₄, filtered, the filtrate was concentrated under reduced pressure and the residue was purified by silica gel column chromatography to give the product (87 g, 34% yield) as a solid. LCMS (ESI) m/z [M+H] calcd for C₁₅H₂₁NO₄: 280.15; found: 280.1.

### Step 2: Synthesis of 3-(5-bromo-2-[2-[(1 S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate

To a mixture of 5-[2-[(1 S)-1-methoxyethyl]pyridin-3-yl]-2,2-dimethyl-5-oxopentanoic acid (78 g, 279 mmol) in EtOH (0.78 L) at room temperature under an atmosphere of N₂ was added (4-bromophenyl)hydrazine HCl salt (68.7 g, 307 mmol) in portions. The mixture was heated to 85° C. and stirred for 2 h, cooled to room temperature, then 4M HCl in dioxane (69.8 mL, 279 mmol) added dropwise. The mixture was heated to 85° C. and stirred for an additional 3 h, then concentrated under reduced pressure and the residue was dissolved in TFA (0.78 L). The mixture was heated to 60° C. and stirred for 1.5 h, concentrated under reduced pressure and the residue adjusted to pH ˜5 with sat. aq. NaHCO₃, then extracted with EtOAc (3×1.5 L). The combined organic layers were dried over anhydrous Na₂SO₄, filtered, the filtrate concentrated under reduced pressure and the residue was purified by silica gel column chromatography to the product (78 g, crude). LCMS (ESI) m/z [M+H] calcd for C₂₁H₂₃BrN₂O₃: 430.1 and C₂₃H₂₇BrN₂O₃: 459.12; found: 431.1 (carboxylic acid) and 459.1.

### Step 3: Synthesis of ethyl 3-(5-bromo-1-ethyl-2-[2-[(1 S)-1-methoxyethyl]pyridin-3-yl]indol-3-yl)-2,2-dimethylpropanoate

To a mixture of 3-(5-bromo-2-[2-[(1 S)-1-methoxyethyl]pyridin-3-yl]-1H-indol-3-yl)-2,2-dimethylpropanoic acid and ethyl (S)-3-(5-bromo-2-(2-(1-methoxyethyl)pyridin-3-yl)-1H-indol-3-yl)-2,2-dimethylpropanoate (198 g, 459 mmol) in DMF (1.8 L) at 0° C. under an atmosphere of N₂ was added Cs₂CO₃ (449 g, 1.38 mol) in portions. Etl (215 g, 1.38 mmol) in DMF (200 mL) was then added dropwise at 0° C. The mixture was warmed to room temperature and stirred for 4 h then diluted with brine (5 L) and extracted with EtOAc (3×2.5 L). The combined organic layers were washed with brine (2×1.5 L), dried over anhydrous Na₂SO₄ and filtered. The filtrate was concentrated under reduced pressure and the residue was purified by silica gel column chromatography to give the product (160 g, 57% yield) as a solid. LCMS (ESI) m/z [M+H] calcd for C₂₅H₃₁BrN₂O₃: 487.17; found: 487.2.

### Step 4: Synthesis of 3-(5-bromo-1-ethyl-2-(2-((S)-1-methoxyethyl)pyridin-3-yl)-1H-indol-3-yl)-2,2-dimethylpropan-1-ol

To a mixture of ethyl 3-(5-bromo-1-ethyl-2-[2-[(1 S)-1-methoxyethyl]pyridin-3-yl]indol-3-yl)-2,2-dimethylpropanoate (160 g, 328 mmol) in THE (1.6 L) at 0° C. under an atmosphere of N₂ was added LiBH₄ (28.6 g, 1.3 mol). The mixture was heated to 60° C. for 16 h, cooled, and quenched with pre-cooled (0° C.) sat. aq. NH₄Cl (5 L). The mixture was extracted with EtOAc (3×2 L) and the combined organic layers were washed with brine (2×1 L), dried over anhydrous Na₂SO₄ and filtered. The filtrate was concentrated under reduced pressure and the residue was purified by silica gel column chromatography to give to two atropisomers of 3-(5-bromo-1-ethyl-2-(2-((S)-1-methoxyethyl)pyridin-3-yl)-1H-indol-3-yl)-2,2-dimethylpropan-1-ol (as single atropisomers) (60 g, 38% yield) and (40 g, 26% yield) both as solids.

LCMS (ESI) m/z [M+H] calcd for C₂₃H₂₉BrN₂O₂: 445.14; found: 445.2.

### Intermediate F3. Synthesis of (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate

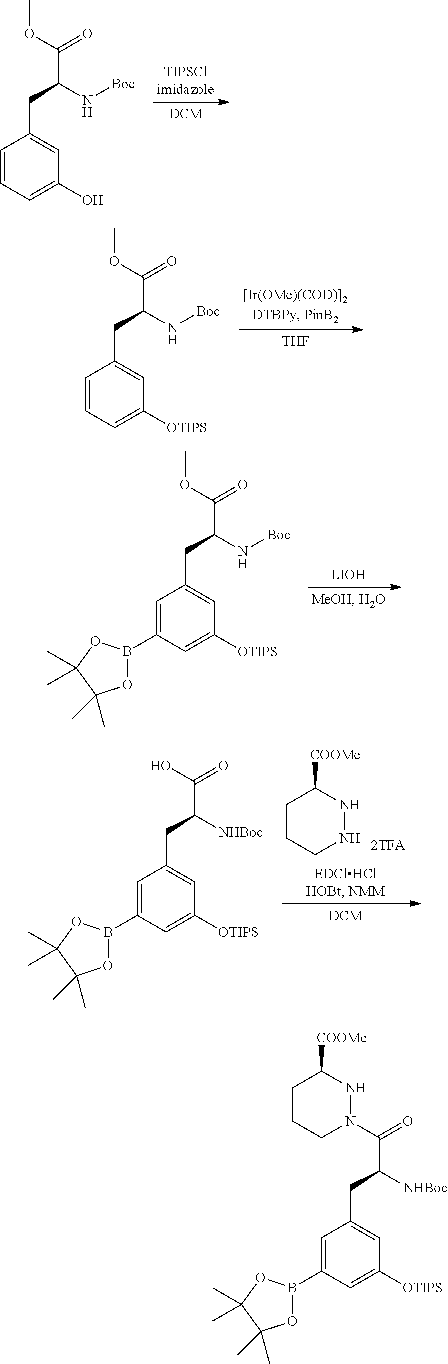

### Step 1: Synthesis of (S)-methyl 2-(tert-butoxycarbonylamino)-3-(3-(triisopropylsilyloxy)phenyl)-propanoate

To a mixture of (S)-methyl 2-(tert-butoxycarbonylamino)-3-(3-hydroxyphenyl)propanoate (10.0 g, 33.9 mmol) in DCM (100 mL) was added imidazole (4.6 g, 67.8 mmol) and TIPSCI (7.8 g, 40.7 mmol). The mixture was stirred at room temperature overnight then diluted with DCM (200 mL) and washed with H₂O (3×150 mL). The organic layer was dried over anhydrous Na₂SO₄, filtered, concentrated under reduced pressure and the residue was purified by silica gel column chromatography to give the product (15.0 g, 98% yield) as an oil. LCMS (ESI) m/z [M+Na] calcd for C₂₄H₄₁NO₅SiNa: 474.22; found: 474.2.

### Step 2: Synthesis of (S)-methyl 2-(tert-butoxycarbonylamino)-3-(3-(4,4,5,5-tetramethyl-1,3,2-dioxaborolan-2-yl)-5-(triisopropylsilyloxy)phenyl)-propanoate

A mixture of (S)-methyl 2-(tert-butoxycarbonylamino)-3-(3-(triisopropylsilyloxy)phenyl)-propanoate (7.5 g, 16.6 mmol), PinB₂ (6.3 g, 24.9 mmol), [Ir(OMe)(COD)]2 (1.1 g, 1.7 mmol) and 4-tert-butyl-2-(4-tert-butyl-2-pyridyl)pyridine (1.3 g, 5.0 mmol) was purged with Ar, then THE (75 mL) was added and the mixture placed under an atmosphere of Ar and sealed. The mixture was heated to 80° C. and stirred for 16 h, concentrated under reduced pressure and the residue was purified by silica gel column chromatography to give the product (7.5 g, 78% yield) as a solid. LCMS (ESI) m/z [M+Na] calcd for C₃₀H₅₂BNO₇SiNa: 600.35; found: 600.4

### Step 3: Synthesis of (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoic acid

To a mixture of triisopropylsilyl (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoate (4.95 g, 6.9 mmol) in MeOH (53 mL) at 0° C. was added LiOH (840 mg, 34.4 mmol) in H₂O (35 mL). The mixture was stirred at 0° C. for 2 h, then acidified to pH ˜5 with 1M HCl and extracted with EtOAc (2×250 mL). The combined organic layers were washed with brine (3×100 mL), dried over anhydrous Na₂SO₄, filtered and the filtrate concentrated under reduced pressure to give the product (3.7 g, 95% yield), which was used directly in the next step without further purification. LCMS (ESI) m/z [M+NH₄] calcd for C₂₉H₅₀BNO₇SiNH₄: 581.38; found: 581.4.

### Step 4: Synthesis of methyl (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate

To a mixture of methyl (S)-hexahydropyridazine-3-carboxylate (6.48 g, 45.0 mmol) in DCM (200 mL) at 0° C. was added NMM (41.0 g, 405 mmol), (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoic acid (24 g, 42.6 mmol) in DCM (50 mL) then HOBt (1.21 g, 9.0 mmol) and EDCI HCl salt (12.9 g, 67.6 mmol). The mixture was warmed to room temperature and stirred for 16 h, then diluted with DCM (200 mL) and washed with H₂O (3×150 mL). The organic layer was dried over anhydrous Na₂SO, filtered, the filtrate concentrated under reduced pressure and the residue was purified by silica gel column chromatography to give the product (22 g, 71% yield) as an oil. LCMS (ESI) m/z [M+H] calcd for C₃₅H₆₀BN₃O₈Si: 690.42; found: 690.5.

### Intermediate F4. Synthesis of Tert-Butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

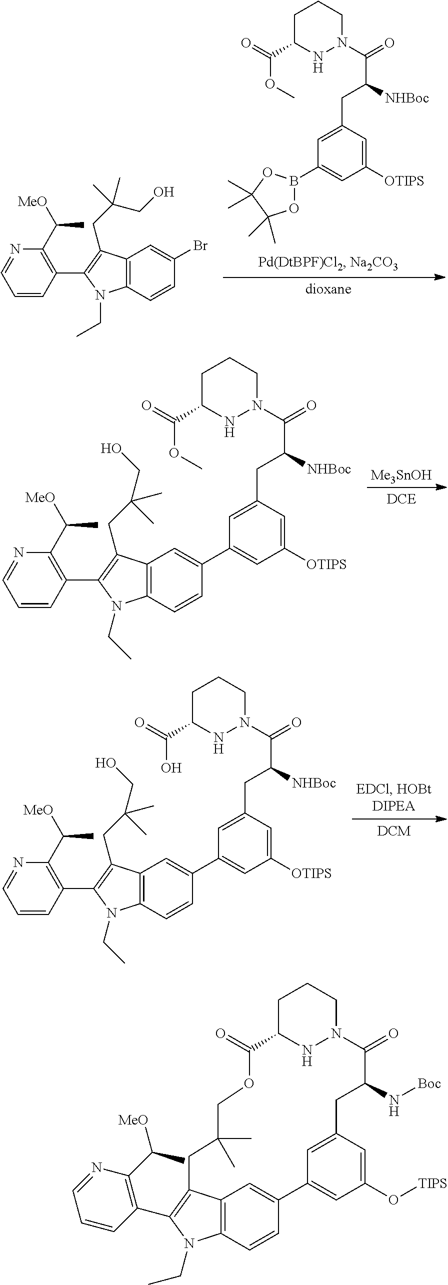

### Step 1: Synthesis of methyl (3S)-1-[(2S)-2-[(tert-butoxycarbonyl)amino]-3-[3-[1-ethyl-3-(3-hydroxy-2,2-dimethylpropyl)-2-[2-[(1 S)-1-methoxyethyl]pyridin-3-yl]indol-5-yl]-5-[(triisopropylsilyl)oxy]phenyl]propanoyl]-1,2-diazinane-3-carboxylate

To a stirred mixture of 3-(5-bromo-1-ethyl-2-[2-[(1 S)-1-methoxyethyl]pyridin-3-yl]indol-3-yl)-2,2-dimethylpropan-1-ol (30 g, 67 mmol) and methyl (3S)-1-[(2S)-2-[(tert-butoxycarbonyl)amino]-3-[3-(4,4,5,5-tetramethyl-1,3,2-dioxaborolan-2-yl)-5-[(triisopropylsilyl)oxy]phenyl]propanoyl]-1,2-diazinane-3-carboxylate (55.8 g, 80.8 mmol) in dioxane (750 mL) at room temperature under an atmosphere of Ar was added Na₂CO₃ (17.9 g, 168.4 mmol), Pd(DtBPF)Cl₂ (4.39 g, 6.7 mmol) and H₂O (150 mL) in portions. The mixture was heated to 85° C. and stirred for 3 h, cooled, diluted with H₂O (2 L) and extracted with EtOAc (3×1 L). The combined organic layers were washed with brine (2×500 mL), dried over anhydrous Na₂SO₄ and filtered. The filtrate was concentrated under reduced pressure and the residue was purified by silica gel column chromatography to give the product (50 g, 72% yield) as a solid. LCMS (ESI) m/z [M+H] calcd for C₅₂H₇₇N₅O₈Si: 928.56; found: 928.8.

### Step 2: Synthesis of (3S)-1-[(2S)-2-[(tert-butoxycarbonyl)amino]-3-[3-[1-ethyl-3-(3-hydroxy-2,2-dimethylpropyl)-2-[2-[(1 S)-1-methoxyethyl]pyridin-3-yl]indol-5-yl]-5-[(triisopropylsilyl)oxy]phenyl]propanoyl]-1,2-diazinane-3-carboxylic acid

To a stirred mixture of methyl (3S)-1-[(2S)-2-[(tert-butoxycarbonyl)amino]-3-[3-[1-ethyl-3-(3-hydroxy-2,2-dimethylpropyl)-2-[2-[(1 S)-1-methoxyethyl]pyridin-3-yl]indol-5-yl]-5-[(triisopropylsilyl)oxy]phenyl]propanoyl]-1,2-diazinane-3-carboxylate (50 g, 54 mmol) in DCE (500 mL) at room temperature was added trimethyltin hydroxide (48.7 g, 269 mmol) in portions. The mixture was heated to 65° C. and stirred for 16 h, then filtered and the filter cake washed with DCM (3×150 mL). The filtrate was concentrated under reduced pressure to give the product (70 g, crude), which was used directly in the next step without further purification. LCMS (ESI) m/z [M+H] calcd for C₅₁H₇₅N₅O₈Si: 914.55; found: 914.6.

### Step 3: Synthesis of tert-butyl ((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a stirred mixture of (3S)-1-[(2S)-2-[(tert-butoxycarbonyl)amino]-3-[3-[1-ethyl-3-(3-hydroxy-2,2-dimethylpropyl)-2-[2-[(1 S)-1-methoxyethyl]pyridin-3-yl]indol-5-yl]-5-[(triisopropylsilyl)oxy]phenyl]propanoyl]-1,2-diazinane-3-carboxylic acid (70 g) in DCM (5 L) at 0° C. under an atmosphere of N₂ was added DIPEA (297 g, 2.3 mol), HOBT (51.7 g, 383 mmol) and EDCI (411 g, 2.1 mol) in portions. The mixture was warmed to room temperature and stirred for 16 h, then diluted with DCM (1 L), washed with brine (3×1 L), dried over anhydrous Na₂SO₄ and filtered. The filtrate was concentrated under reduced pressure and the residue was purified by silica gel column chromatography to give the product (36 g, 42% yield) as a solid. LCMS (ESI) m/z [M+H] calcd for C₅₁H₇₃N₅O₇Si: 896.54; found: 896.5.

### Intermediate F5. Synthesis of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

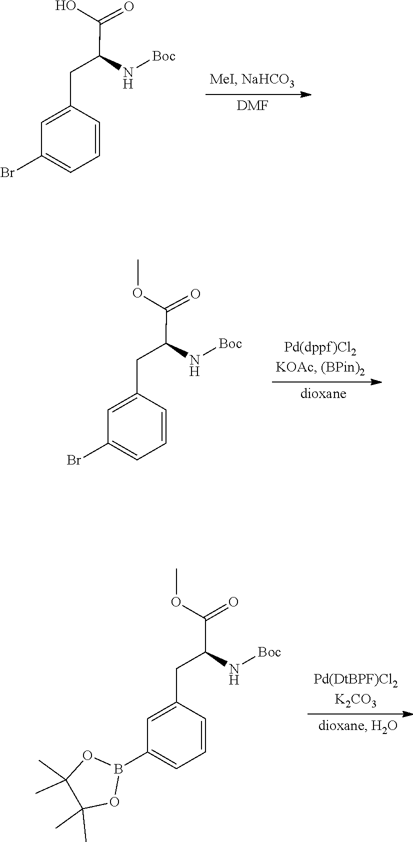

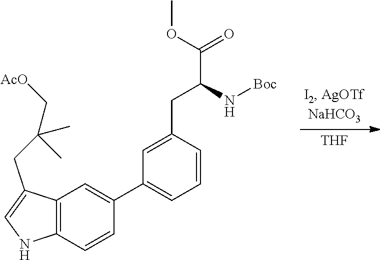 [SMILES: "THF" ; COC(=O)[C@H](Cc1cccc(-c2ccc3[nH]cc(CC(C)(C)COC(C)=O)c3c2)c1)NC(=O)OC(C)(C)C]

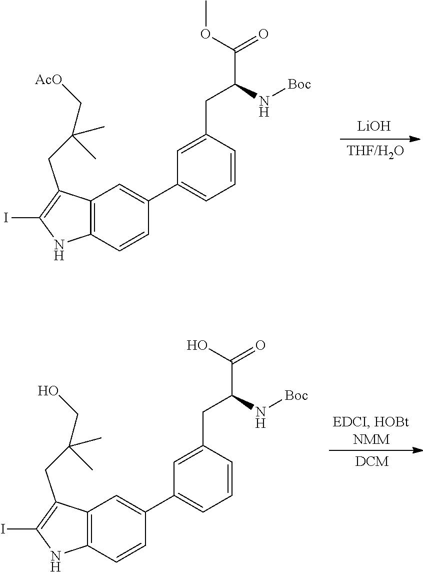

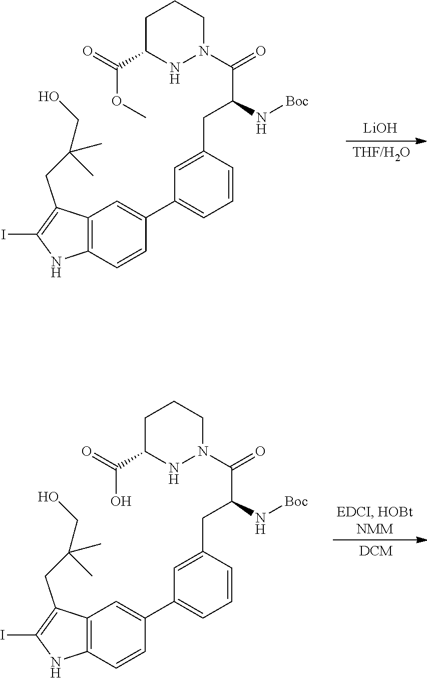

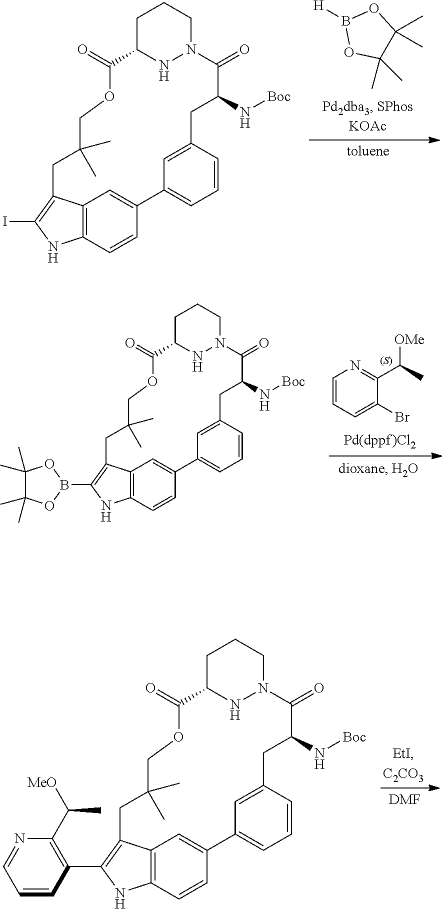

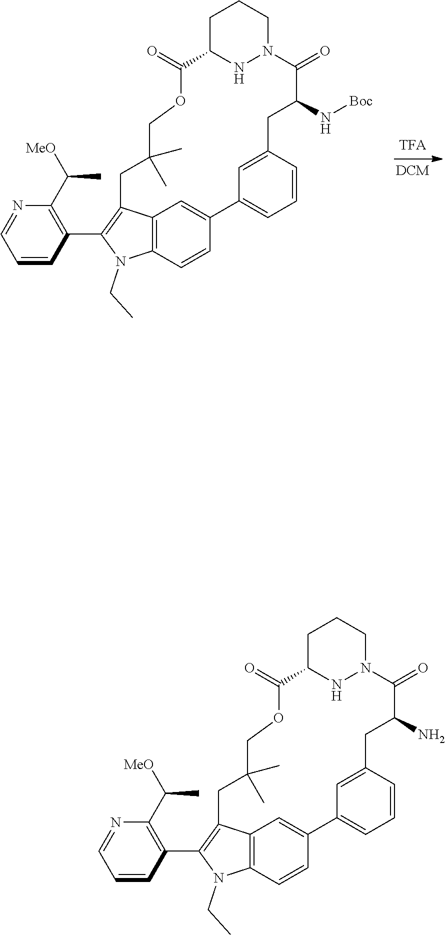

### Step 1: Synthesis of methyl (S)-3-(3-bromophenyl)-2-((tert-butoxycarbonyl)amino)propanoate

To a solution of (2S)-3-(3-bromophenyl)-2-[(tert-butoxycarbonyl)amino]propanoic acid (100 g, 290 mmol) in DMF (1 L) at room temperature was added NaHCO₃ (48.8 g, 581.1 mmol) and Mel (61.9 g, 435.8 mmol). The reaction mixture was stirred for 16 h and was then quenched with H₂O (1 L) and extracted with EtOAc (3×1 L). The combined organic layers were washed with brine (3×500 mL), dried over Na₂SO₄, filtered, and concentrated under reduced pressure. The residue was purified by silica gel column chromatography (13% EtOAc/pet. ether) to give the final product (109 g, crude). LCMS (ESI) m/z [M+Na] calcd for C₁₅H₂₀BrNO₄ 380.05; found: 380.0.

### Step 2: Synthesis of methyl (S)-2-((tert-butoxycarbonyl)amino)-3-(3-(4,4,5,5-tetramethyl-1,3,2-dioxaborolan-2-yl)phenyl)propanoate

To a stirred solution of methyl (2S)-3-(3-bromophenyl)-2-[(tert-butoxycarbonyl)amino]propanoate (108 g, 301.5 mmol) and bis(pinacolato)diboron (99.53 g, 391.93 mmol) in dioxane (3.2 L) was added KOAc (73.97 g, 753.70 mmol) and Pd(dppf)Cl₂ (22.06 g, 30.15 mmol). The reaction mixture was heated to 90° C. for 3 h and was then cooled to room temperature and extracted with EtOAc (2×3 L). The combined organic layers were washed with brine (3×800 mL), dried over Na₂SO₄, filtered, and concentrated under reduced pressure. The residue was purified by silica gel column chromatography (5% EtOAc/pet. ether) to afford the product (96 g, 78.6% yield). LCMS (ESI) m/z [M+Na] calcd for C₂₁H₃₂BNO₆ 428.22; found: 428.1.

### Step 3: Synthesis of methyl (S)-3-(3-(3-(3-acetoxy-2,2-dimethylpropyl)-1H-indol-5-yl)phenyl)-2-((tert-butoxycarbonyl)amino)propanoate

To a mixture of methyl (2S)-2-[(tert-butoxycarbonyl)amino]-3-[3-(4,4,5,5-tetramethyl-1,3,2-dioxaborolan-2-yl)phenyl]propanoate (94 g, 231.9 mmol) and 3-(5-bromo-1H-indol-3-yl)-2,2-dimethylpropyl acetate (75.19 g, 231.93 mmol) in dioxane (1.5 L) and H₂O (300 mL) was added K₂CO₃ (64.11 g, 463.85 mmol) and Pd(DtBPF)Cl₂ (15.12 g, 23.19 mmol). The reaction mixture was heated to 70° C. and stirred for 4 h. The reaction mixture was extracted with EtOAc (2×2 L) and the combined organic layers were washed with brine (3×600 mL), dried over Na₂SO₄, filtered, and concentrated under reduced pressure. The residue was purified by silica gel column chromatography (20% EtOAc/pet. ether) to give the product (130 g, crude). LCMS (ESI) m/z [M+H] calcd for C₃₀H₃₈N₂O₆ 523.28; found: 523.1.

### Step 4: Synthesis of methyl (S)-3-(3-(3-(3-acetoxy-2,2-dimethylpropyl)-2-iodo-1H-indol-5-yl)phenyl)-2-((tert-butoxycarbonyl)amino)propanoate

To a solution of methyl (2S)-3-(3-[3-[3-(acetyloxy)-2,2-dimethylpropyl]-1H-indol-5-yl]phenyl)-2-[(tert-butoxycarbonyl)amino]propanoate (95.0 g, 181.8 mmol) and iodine (36.91 g, 145.41 mmol) in THE (1 L) at −10° C. was added AgOTf (70.0 g, 272.7 mmol) and NaHCO₃(22.9 g, 272.65 mmol). The reaction mixture was stirred for 30 min and was then quenched by the addition of sat. aq. Na₂S₂O₃ (100 mL) at 0° C. The resulting mixture was extracted with EtOAc (3×1 L) and the combined organic layers were washed with brine (3×500 mL), dried over Na₂SO₄, filtered, and concentrated under reduced pressure. The residue was purified by silica gel column chromatography (50% EtOAc/pet. ether) to give methyl (S)-3-(3-(3-(3-acetoxy-2,2-dimethylpropyl)-2-iodo-1H-indol-5-yl)phenyl)-2-((tert-butoxycarbonyl)amino)propanoate (49.3 g, 41.8% yield). LCMS (ESI) m/z [M+H] calcd for C₃₀H₃₇IN₂O₆: 649.18; found: 649.1.

### Step 5: Synthesis of (S)-2-((tert-butoxycarbonyl)amino)-3-(3-(3-(3-hydroxy-2,2-dimethylpropyl)-2-iodo-1H-indol-5-yl)phenyl)propanoic acid

To a solution of methyl (2S)-3-(3-[3-[3-(acetyloxy)-2,2-dimethylpropyl]-2-iodo-1H-indol-5-yl]phenyl)-2-[(tert-butoxycarbonyl)amino]propanoate (60 g, 92.5 mmol) in THE (600 mL) was added a solution of LiOH⋅H₂O (19.41 g, 462.5 mmol) in H₂O (460 mL). The resulting solution was stirred overnight and then the pH was adjusted to 6 with HCl (1 M). The resulting solution was extracted with EtOAc (2×500 mL) and the combined organic layers was washed with brine (2×500 mL), dried over Na₂SO₄, filtered, and concentrated under reduced pressure to give the product (45 g, 82.1% yield). LCMS (ESI) m/z [M+Na] calcd for C₂₇H₃₃IN₂O₆ 615.13; found: 615.1.

### Step 6: Synthesis of methyl (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylate

To a solution of (2S)-2-[(tert-butoxycarbonyl)amino]-3-[3-[3-(3-hydroxy-2,2-dimethylpropyl)-2-iodo-1H-indol-5-yl]phenyl]propanoic acid (30 g, 50.6 mmol) and methyl (3S)-1,2-diazinane-3-carboxylate (10.9 g, 75.9 mmol) in DCM (400 mL) was added NMM (40.97 g, 405.08 mmol), HOBt (2.05 g, 15.19 mmol), and EDCI (19.41 g, 101.27 mmol). The reaction mixture was stirred overnight and then the mixture was washed with sat. aq. NH₄Cl (2×200 mL) and brine (2×200 mL), and the mixture was dried over Na₂SO₄, filtered, and concentrated under reduced pressure to give the product (14 g, 38.5% yield).

LCMS (ESI) m/z [M+H] calcd for C₃₃H₄₃IN₄O₆ 718.23; found: 719.4.

### Step 7: Synthesis of (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylic acid

To a solution of methyl (S)-1-((S)-2-((tert-butoxycarbonyl)amino)-3-(3-(3-(3-hydroxy-2,2-dimethylpropyl)-2-iodo-1H-indol-5-yl)phenyl)propanoyl)hexahydropyridazine-3-carboxylate (92 g, 128.0 mmol) in THE (920 mL) at 0° C. was added a solution of LiOH⋅H₂O (26.86 g, 640.10 mmol) in H₂O (640 mL). The reaction mixture was stirred for 2 h and was then concentrated under reduced pressure to give the product (90 g, crude). LCMS (ESI) m/z [M+H] calcd for C₃₂H₄₁IN₄O₆ 705.22; found: 705.1.

### Step 8: Synthesis of tert-butyl ((63S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of (3S)-1-[(2S)-2-[(tert-butoxycarbonyl)amino]-3-[3-[3-(3-hydroxy-2,2-dimethylpropyl)-2-iodo-1H-indol-5-yl]phenyl]propanoyl]-1,2-diazinane-3-carboxylic acid (90 g, 127.73 mmol) in DCM (10 L) at 0° C. was added HOBt (34.52 g, 255.46 mmol), DIPEA (330.17 g, 2554.62 mmol) and EDCI (367.29 g, 1915.96 mmol). The reaction mixture was stirred for 16 h and was then concentrated under reduced pressure. The mixture was extracted with DCM (2×2 L) and the combined organic layers were washed with brine (3×1 L), dried over Na₂SO₄, filtered, and concentrated under reduced pressure. The residue was purified by silica gel column chromatography (50% EtOAc/pet. ether) to give the product (70 g, 79.8% yield). LCMS (ESI) m/z [M+H] calcd for C₃₂H₃₉IN₄O₅ 687.21; found: 687.1.

### Step 9: Synthesis of tert-butyl ((63S,4S)-10,10-dimethyl-5,7-dioxo-12-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

A 1 L round-bottom flask was charged with tert-butyl ((63S,4S)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (22.0 g, 32.042 mmol), toluene (300.0 mL), Pd₂(dba)₃ (3.52 g, 3.845 mmol), S-Phos (3.95 g, 9.613 mmol), and KOAc (9.43 g, 96.127 mmol) at room temperature. To the mixture was added 4,4,5,5-tetramethyl-1,3,2-dioxaborolane (26.66 g, 208.275 mmol) dropwise with stirring at room temperature. The resulting solution was stirred for 3 h at 60° C. The resulting mixture was filtered, and the filter cake was washed with EtOAc. The filtrate was concentrated under reduced pressure and the remaining residue was purified by silica gel column chromatography to afford the product (22 g, 90% yield) as a solid. LCMS (ESI) m/z [M+H] calcd for C₃₈H₅₁BN₄O₇ 687.3; found: 687.4.

### Step 10: Synthesis of tert-butyl ((6³S,4S)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

A mixture of tert-butyl ((63S,4S)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (2.0 g, 2.8 mmol), 3-bromo-2-[(1 S)-1-methoxyethyl]pyridine (0.60 g, 2.8 mmol), Pd(dppf)Cl₂ (0.39 g, 0.5 mmol), and K₃PO₄ (1.2 g, 6.0 mmol) in dioxane (50 mL) and H₂O (10 mL) under an atmosphere of N₂ was heated to 70° C. and stirred for 2 h. The mixture was diluted with H₂O (50 mL) and extracted with EtOAc (3×50 mL). The combined organic layers were washed with brine (3×50 mL), dried over anhydrous Na₂SO₄, and filtered. The filtrate was concentrated under reduced pressure and the residue was purified by silica gel column chromatography to afford the product (1.5 g, 74% yield) as a solid. LCMS (ESI) m/z [M+H] calcd for C₄₀H₄₉N₅O₆ 695.4; found: 696.5.

### Step 11: Synthesis of tert-butyl ((63S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of tert-butyl ((63S,4S)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl) carbamate (20 g, 28.7 mmol) and Cs₂CO₃ (18.7 g, 57.5 mmol) in DMF (150 mL) at 0° C. was added a solution of Etl (13.45 g, 86.22 mmol) in DMF (50 mL). The resulting mixture was stirred overnight at 35° C. and then diluted with H₂O (500 mL). The mixture was extracted with EtOAc (2×300 mL) and the combined organic layers were washed with brine (3×100 mL), dried over Na₂SO₄, filtered, and concentrated under reduced pressure. The residue was purified by silica gel column chromatography to afford the product (4.23 g, 18.8% yield) and the atropisomer (5.78 g, 25.7% yield) as solids. LCMS (ESI) m/z [M+H] calcd for C₄₂H₅₃N₅O₆ 724.4; found: 724.6.

### Step 12: Synthesis of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

A mixture of tert-butyl ((63S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.3 g, 1.7 mmol) in TFA (10 mL) and DCM (20 mL) was stirred at 0° C. for 2 h. The mixture was concentrated under reduced pressure to afford the product (1.30 g, crude) as a solid. LCMS (ESI) m/z [M+H] calcd for C₃₇H₄₅N₅O₄ 623.3; found: 624.4.

### Intermediate F6. Synthesis of (6³S,4S)-4-amino-1¹-ethyl-2⁵-(fluoromethyl)-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

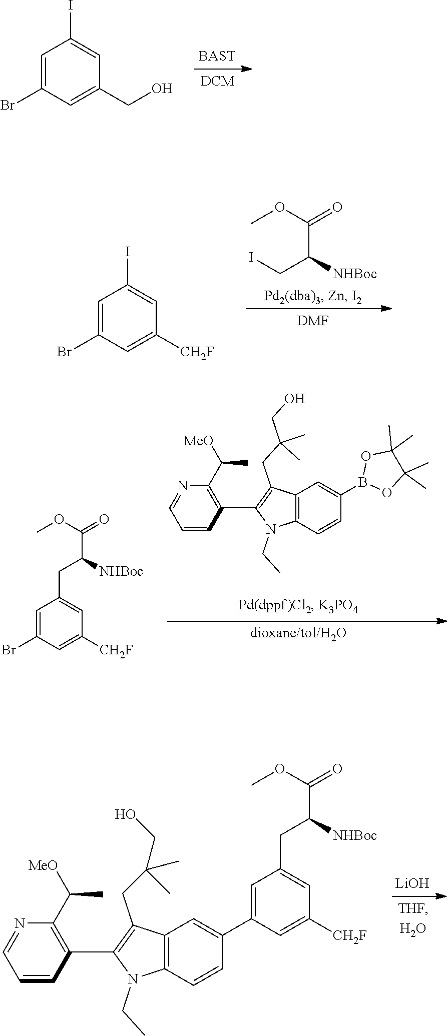

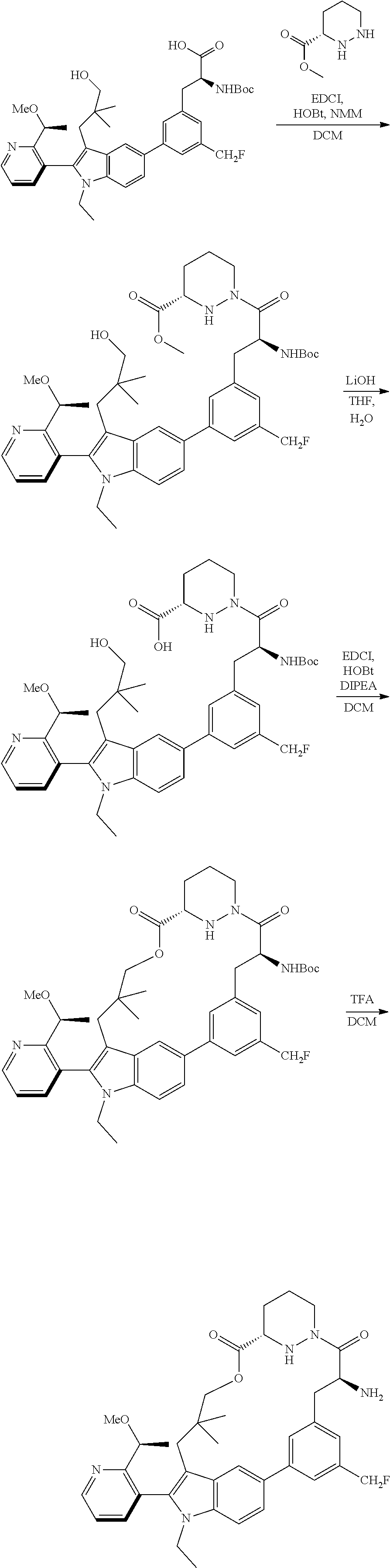

### Step 1: Synthesis of 1-bromo-3-(fluoromethyl)-5-iodobenzene

To a solution of (3-bromo-5-iodophenyl)methanol (175.0 g, 559.23 mmol) in DCM (2 L) was added BAST (247.45 g, 1118.45 mmol) dropwise at 0° C. The resulting mixture was stirred for 16 h at room temperature. The reaction was quenched with sat. aq. NaHCO₃ at 0° C. The organic layers were washed with H₂O (3×700 mL) and dried over anhydrous Na₂SO₄. After filtration, the filtrate was concentrated under reduced pressure. The residue was purified by silica gel column chromatography (3% EtOAc/pet. ether) to afford the desired product (120 g, 68% yield).

Step 2: Synthesis of methyl (2S)-3-[3-bromo-5-(fluoromethyl)phenyl]-2-[(tert-butoxycarbonyl)amino]propanoate

Into a 1000 mL 3-necked round-bottom flask was added Zn powder (32.40 g, 495.358 mmol) in DMF (350.0 mL) and I₂ (967.12 mg, 3.810 mmol). To the mixture was added a solution of methyl (2R)-2-[(tert-butoxycarbonyl)amino]-3-iodopropanoate (27.0 g, 82.03 mmol) in DMF (10 mL). The mixture was heated to 30° C. for 10 min. To the mixture was then added a solution of methyl (2R)-2-[(tert-butoxycarbonyl)amino]-3-iodopropanoate (54.0 g, 164.07 mmol) in DMF (20 mL). The resulting mixture was stirred for 30 min at room temperature and filtered. The resulting solution was added to a mixture of 1-bromo-3-(fluoromethyl)-5-iodobenzene (60 g, 190.522 mmol), tris(furan-2-yl)phosphane (2.65 g, 11.431 mmol), and Pd₂(dba)₃ (3.49 g, 3.810 mmol) in DMF (400 mL) at room temperature under an argon atmosphere and the reaction mixture was heated to 60° C. for 10 min then removed the oil bath. The resulting mixture was stirred for about 1 h until the temperature cooled to 50° C. The reaction was quenched with sat. aq. NH₄Cl (3000 mL) and the resulting mixture was extracted with EtOAc (3×1000 mL). The combined organic layers were washed with brine (2×1000 mL) and dried over anhydrous Na₂SO₄. After filtration, the filtrate was concentrated under reduced pressure. The residue was purified by silica gel column chromatography (9% EtOAc/pet. ether) to afford the desired product (45 g, 60% yield).

### Step 3: Synthesis of methyl (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoate

A mixture of methyl (2S)-3-[3-bromo-5-(fluoromethyl)phenyl]-2-[(tert-butoxycarbonyl)amino]propanoate (75.28 g, 192.905 mmol), (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (95 g, 192.905 mmol), Pd(dppf)Cl₂ (14.11 g, 19.291 mmol) and K₂CO₃ (53.32 g, 385.810 mmol) in dioxane (900 mL) and H₂O (180 mL) was stirred for 2 h at 80° C. The resulting mixture was concentrated under reduced pressure and was then diluted with H₂O. The resulting mixture was extracted with EtOAc (3×1200 mL) and the combined organic layers were washed with H₂O (3×500 mL) and dried over anhydrous Na₂SO₄. After filtration, the filtrate was concentrated under reduced pressure. The residue was purified by silica gel column chromatography (50% EtOAc/pet. ether) to afford the desired product (105 g, 80% yield). LCMS (ESI) m/z [M+H] calcd for C₃₉H₅₀FN₃O₆: 676.38; found: 676.1.

### Step 4: Synthesis of (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoic acid

To a stirred solution of methyl (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoate (108 g, 159.801 mmol) in THE (500 mL) was added a solution of LiOH⋅H₂O (11.48 g, 479.403 mmol) in H₂O (500 mL) at 0° C. The resulting mixture was stirred for 2 h at 0° C. and was then acidified to pH 6 with 1 M HCl (aq.). The mixture was extracted with EtOAc (3×800 mL) and the combined organic layers were washed with brine (2×200 mL) and dried over anhydrous Na₂SO₄. After filtration, the filtrate was concentrated under reduced pressure to afford the desired product (101 g, crude). LCMS (ESI) m/z [M+H] calcd for C₃₈H₄₈FN₃O₆: 662.36; found: 662.1.

### Step 5: Synthesis of methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylate

To a stirred solution of (S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoic acid (103 g, 155.633 mmol) and NMM (157.42 g, 1556.330 mmol) in DCM (1200 mL) was added methyl (3S)-1,2-diazinane-3-carboxylate (33.66 g, 233.449 mmol), HOBt (10.51 g, 77.816 mmol) and EDCI (59.67 g, 311.265 mmol) in portions at 0° C. The resulting mixture was stirred at room temperature for 16 h. The organic layers were then washed with 0.5 M HCl (2×1000 mL) and brine (2×800 mL), dried over anhydrous Na₂SO₄, filtered, and concentrated under reduced pressure. The residue was purified by silica gel column chromatography (50% EtOAc/pet. ether) to afford the desired product (103 g, 83% yield).

LCMS (ESI) m/z [M+H] calcd for C₄₄H₅₈FN₅O₇: 788.44; found: 788.1.

### Step 6: Synthesis of (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylic acid

To a stirred solution of methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylate (103 g, 130.715 mmol) in THE (700 mL) was added a solution of LiOH⋅H₂O (27.43 g, 653.575 mmol) in H₂O (700 mL) at 0° C. The resulting mixture was stirred for 2 h at 0° C. and was then neutralized to pH 6 with 1 M HCl. The resulting mixture was extracted with EtOAc (3×800 mL) and the combined organic layers were washed with brine (2×600 mL), dried over anhydrous Na₂SO₄, filtered, and concentrated under reduced pressure to afford the desired product (101 g, crude). LCMS (ESI) m/z [M+H] calcd for C₄₃H₅₆FN₅O₇: 774.43; found: 774.1.

### Step 7: Synthesis of tert-butyl ((63S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a stirred solution of (S)-1-((S)-2-((tert-butoxycarbonyl)amino)-3-(3-(1-ethyl-3-(3-hydroxy-2,2-dimethylpropyl)-2-(2-((S)-1-methoxyethyl)pyridin-3-yl)-1H-indol-5-yl)-5-(fluoromethyl)phenyl)propanoyl)hexahydropyridazine-3-carboxylic acid (101 g, 130.50 mmol) in DCM (5500 mL) was added DIPEA (227.31 mL, 1305.0 mmol) HOBt (88.17 g, 652.499 mmol), and EDCI (375.26 g, 1957.498 mmol) at 0° C. The resulting mixture was stirred at room temperature overnight. The mixture was then washed with 0.5 M HCl (2×2000 mL), brine (2×2000 mL), dried over anhydrous Na₂SO₄, filtered, and concentrated under reduced pressure. The residue was purified by silica gel column chromatography (50% EtOAc/pet. ether) to afford the desired product (68 g, 65% yield). LCMS (ESI) m/z [M+H] calcd for C₄₃H₅₄FN₅O₆: 756.42; found: 756.4.

### Step 8: Synthesis of (2S)—N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-(methylamino)butanamide

To a stirred solution of tert-butyl ((63S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (350 mg, 0.403 mmol) in DCM (4 mL) was added TFA (1.5 mL) at 0° C. The resulting mixture was stirred at room temperature for 1.5 h and was then concentrated under reduced pressure to afford the desired product (600 mg, crude). LCMS (ESI) m/z [M+H] calcd for C₃₈H₄₆FN₅O₄: 656.36; found: 656.4.

### Intermediate F7. Synthesis of (6³S,4S)-4-amino-2⁵-(difluoromethyl)-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

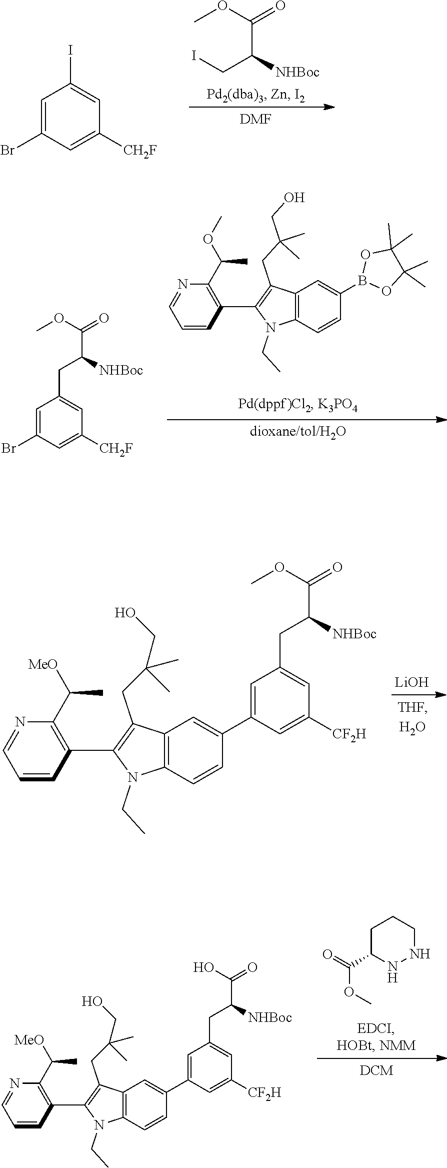

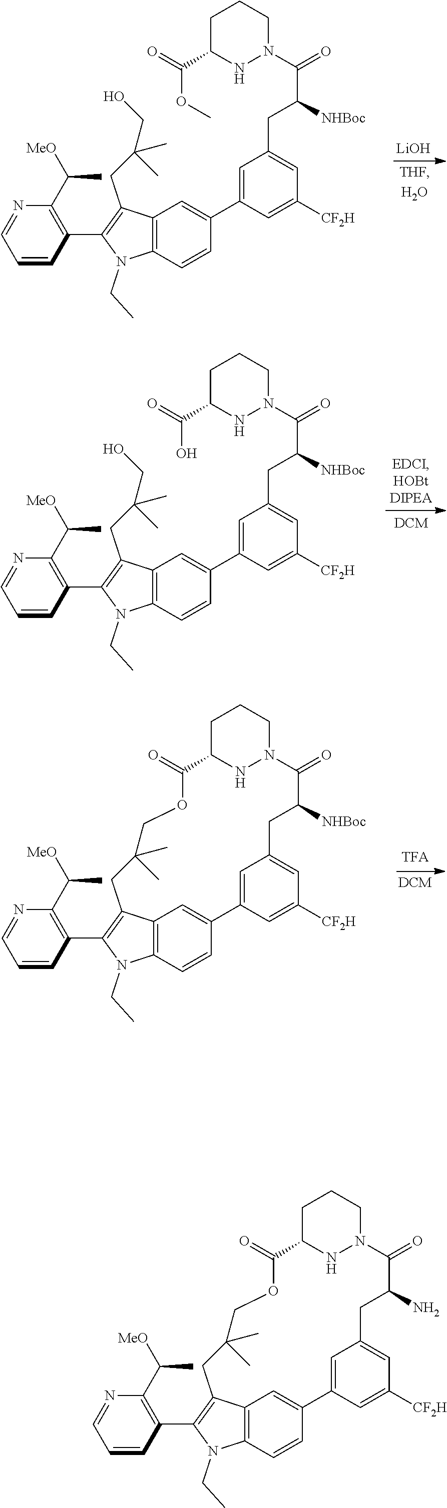

### Step 1: Synthesis of methyl (S)-3-(3-bromo-5-(difluoromethyl)phenyl)-2-((tert-butoxycarbonyl)amino)propanoate

Into a 1000 mL 3-necked round-bottom flask was added Zn powder (43.42 g, 663.835 mmol) and 12 (1.30 g, 5.106 mmol) in DMF (400 mL) at room temperature. To the above mixture was added a solution of methyl (2R)-2-[(tert-butoxycarbonyl)amino]-3-iodopropanoate (36.42 g, 110.64 mmol) in DMF (10 mL). The mixture was heated to 30° C. for 10 min. To the mixture was then added a solution of methyl (2R)-2-[(tert-butoxycarbonyl)amino]-3-iodopropanoate (72.83 g, 221.28 mmol) in DMF (20 mL) dropwise at room temperature. The resulting mixture was stirred for 30 min. The mixture was filtered and the solution was added to a mixture of 1-bromo-3-(difluoromethyl)-5-iodobenzene (85.0 g, 255.321 mmol), tris(furan-2-yl) phosphane (3.56 g, 15.319 mmol), and Pd₂(dba)₃ (4.68 g, 5.106 mmol) in DMF (400 mL) at room temperature under an argon atmosphere. The reaction mixture was heated to 60° C. for 10 min and then removed from the oil bath and was stirred for 1 h until the temperature of the resulting mixture cooled to 50° C. The reaction was quenched with sat. aq. NH₄Cl (3000 mL) and the aqueous layer was extracted with EtOAc (3×1000 mL). The combined organic layers were washed with brine (2×1000 mL), dried over anhydrous Na₂SO₄, filtered, and concentrated under reduced pressure. The residue was purified by silica gel column chromatography (9% EtOAc/pet. ether) to afford the desired product (59 g, 56.6% yield).

### Step 2: Synthesis of methyl (S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoate

A mixture of methyl (2S)-3-[3-bromo-5-(difluoromethyl)phenyl]-2-[(tert-butoxycarbonyl)amino]propanoate (90.0 g, 220.459 mmol), (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (1.50 g, 3.046 mmol), Pd(dppf)Cl₂ (16.13 g, 22.046 mmol) and K₃PO₄ (116.99 g, 551.148 mmol) in dioxane (600 mL), H₂O (200 mL), and toluene(200 mL) was stirred for 2 h at 70° C. The resulting mixture was concentrated under reduced pressure and then diluted with H₂O (300 mL). The mixture was extracted with EtOAc (3×500 mL). The combined organic layers were washed with H₂O (3×500 mL), dried over anhydrous Na₂SO₄. After filtration, the filtrate was concentrated under reduced pressure and the residue was purified by silica gel column chromatography (50% EtOAc/pet. ether) to afford the desired product (128 g, 83.7% yield).

LCMS (ESI) m/z [M+H] calcd for C₃₉H₄₉F₂N₃O₆: 694.37; found: 694.2.

### Step 3: Synthesis of (S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoic acid

To a stirred solution of methyl (S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoate (125.0 g, 180.159 mmol) in THE (800 mL) was added LiOH⋅H₂O (11.48 g, 479.403 mmol) in H₂O (200 mL) dropwise at 0° C. The resulting mixture was stirred for 2 h at 0° C. The mixture was acidified to pH 6 with 1 M HCl (aq.) and then extracted with EtOAc (3×800 mL). The combined organic layers were washed with brine (2×200 mL), dried over anhydrous Na₂SO₄. After filtration, the filtrate was concentrated under reduced pressure to afford the desired product (125 g, crude). LCMS (ESI) m/z [M+H] calcd for C₃₈H₄₇F₂N₃O₆: 680.37; found: 680.2.

### Step 4: Synthesis of methyl (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylate

To a stirred solution of methyl (3S)-1,2-diazinane-3-carboxylate (39.77 g, 275.814 mmol) and NMM (185.98 g, 1838.760 mmol) in DCM (1500 mL) was added (S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoic acid (125.0 g, 183.876 mmol), HOBt (12.42 g, 91.938 mmol) and EDCI (70.50 g, 367.752 mmol) in portions at 0° C. The resulting mixture was stirred at room temperature for 16 h. The reaction mixture was then washed with 0.5 M HCl (2×1000 mL) and brine (2×800 mL), dried over anhydrous Na₂SO₄, filtered, and concentrated under reduced pressure. The residue was purified by silica gel column chromatography (50% EtOAc/pet.ether) to afford the desired product (110 g, 74.2% yield).

LCMS (ESI) m/z [M+H] calcd for C₄₄H₅₇F₂N₅O₇: 806.43; found: 806.2.

### Step 5: Synthesis of (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylic acid

To a stirred solution of methyl (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylate (110.0 g, 136.482 mmol) in THE (800 mL) was added a solution of LiOH⋅H₂O (17.18 g, 409.446 mmol) in H₂O (200 mL) in portions at 0° C. The resulting mixture was stirred for 2 h at 0° C. and was then neutralized to pH 6 with 0.5 M HCl. The resulting mixture was extracted with EtOAc (3×800 mL) and the combined organic layers were washed with brine (2×600 mL), dried over anhydrous Na₂SO₄, filtered, and concentrated under reduced pressure to afford the desired product (100 g, crude). LCMS (ESI) m/z [M+H] calcd for C₄₃H₅₅F₂N₅O₇: 792.42; found: 792.4.

### Step 6: Synthesis of tert-butyl ((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)carbamate

To a stirred solution of (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)pyridin-3-yl)-1H-indol-5-yl)phenyl)propanoyl)hexahydropyridazine-3-carboxylic acid (100.0 g, 126.273 mmol) in DCM (6000 mL) was added DIPEA (163.20 g, 1262.730 mmol), HOBt (85.31 g, 631.365 mmol), and EDCI (363.10 g, 1894.095 mmol) dropwise at 0° C. The resulting mixture was stirred overnight at room temperature. The mixture was then washed with 0.5 M HCl (2×2 000 mL) and brine (2×2000 mL), dried over anhydrous Na₂SO₄, filtered, and concentrated under reduced pressure. The residue was purified by silica gel column chromatography (50% EtOAc/pet. ether) to afford the desired product (70 g, 71.6% yield). LCMS (ESI) m/z [M+H] calcd for C₄₃H₅₃F₂N₅O₆: 774.41; found: 774.0.

### Step 7: Synthesis of (6³S,4S)-4-amino-2⁵-(difluoromethyl)-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a stirred solution of tert-butyl ((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)carbamate (202.0 mg, 0.261 mmol) in DCM (2 mL) was added TFA (1.0 mL) dropwise at 0° C. The resulting mixture was stirred for 1.5 h at 0° C. and was then concentrated under reduced pressure to afford the desired product. LCMS (ESI) m/z [M+H] calcd for C₃₈H₄₅F₂N₅O₄: 674.35; found: 674.5.

### Intermediate F8. Synthesis of Tert-Butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

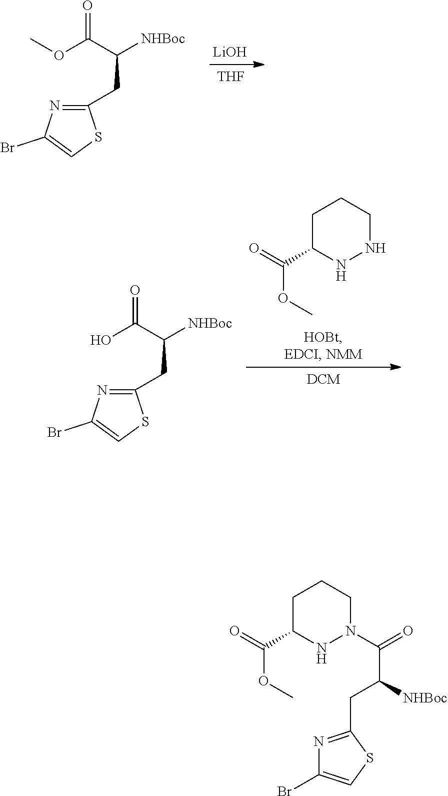

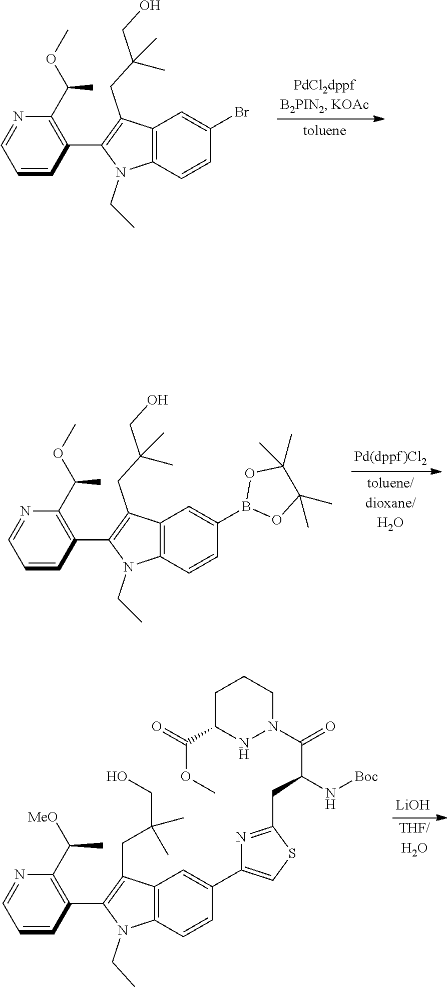

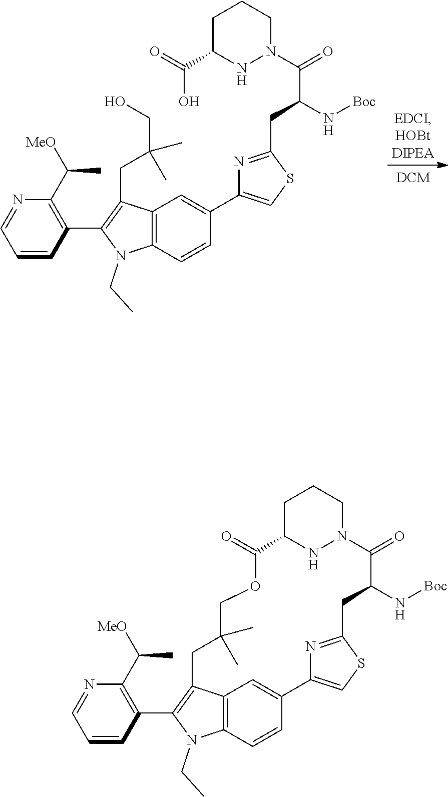

### Step 1: Synthesis of (S)-3-(4-bromothiazol-2-yl)-2-((tert-butoxycarbonyl)amino)propanoic acid

To a solution of methyl (2S)-3-(4-bromo-1,3-thiazol-2-yl)-2-[(tert-butoxycarbonyl)amino]propanoate (110 g, 301.2 mmol) in THE (500 mL) and H₂O (200 mL) at room temperature was added LiOH (21.64 g, 903.6 mmol). The resulting solution was stirred for 1 h and then concentrated under reduced pressure. The resulting residue was adjusted to pH 6 with 1 M HCl and then extracted with DCM (3×500 mL). The combined organic layers were, dried over Na₂SO₄, filtered, and concentrated under reduced pressure to afford the desired product (108 g, crude). LCMS (ESI) m/z [M+H] calcd for C₁₁H₁₅BrN₂O₄S: 351.00; found: 351.0.

### Step 2: Synthesis of methyl (S)-1-((S)-3-(4-bromothiazol-2-yl)-2-((tert-butoxycarbonyl)amino)propanoyl)hexahydropyridazine-3-carboxylate

To a solution of (S)-3-(4-bromothiazol-2-yl)-2-((tert-butoxycarbonyl)amino)propanoic acid (70 g, 199.3 mmol) in DCM (500 mL) at 0° C. was added methyl (3S)-1,2-diazinane-3-carboxylate bis(trifluoroacetic acid) salt (111.28 g, 298.96 mmol), NMM (219.12 mL. 1993.0 mmol), EDCI (76.41 g, 398.6 mmol) and HOBt (5.39 g, 39.89 mmol). The resulting solution was warmed to room temperature and stirred for 1 h. The reaction was then quenched with H₂O (500 mL) and was extracted with EtOAc (3 20×500 mL). The combined organic layers were dried over Na₂SO₄, filtered, and concentrated under reduced pressured. The residue was purified by silica gel column chromatography (0→50% EtOAc/pet. ether) to afford the desired product (88.1 g, 93% yield). LCMS (ESI) m/z [M+H] calcd for C₁₇H₂₅BrN₄O₅S: 477.08; found: 477.1.

### Step 3: Synthesis of (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol

To a solution of 3-(5-bromo-1-ethyl-2-(2-((S)-1-methoxyethyl)pyridin-3-yl)-1H-indol-3-yl)-2,2-dimethylpropan-1-ol (60 g, 134.7 mmol) in toluene (500 mL) at room temperature was added bis(pinacolato)diboron (51.31 g, 202.1 mmol), Pd(dppf)Cl₂ (9.86 g, 13.48 mmol) and KOAc (26.44 g, 269.4 mmol). Then reaction mixture was then heated to 90° C. and stirred for 2 h. The reaction solution was then cooled to room temperature and concentrated under reduced pressure. Purification by silica gel column chromatography (0→50% EtOAc/pet. ether) afforded the desired product (60.6 g, 94% yield).

LCMS (ESI) m/z [M+H] calcd for C₂₉H₄₁BN₂O₄: 493.32; found: 493.3.

### Step 4: Synthesis of methyl (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylate

To a solution of (S)-3-(1-ethyl-2-(2-(1-methoxyethyl)pyridin-3-yl)-5-(4,4,5,5-tetramethyl-1,3,2-dioxaborolan-2-yl)-1H-indol-3-yl)-2,2-dimethylpropan-1-ol (30 g, 60.9 mmol) in toluene (600 mL), dioxane (200 mL), and H₂O (200 mL) at room temperature was added methyl (S)-1-((S)-3-(4-bromothiazol-2-yl)-2-((tert-butoxycarbonyl)amino)propanoyl)hexahydropyridazine-3-carboxylate (43.62 g, 91.4 mmol), K₃PO₄ (32.23 g, 152.3 mmol) and Pd(dppf)Cl₂ (8.91 g, 12.18 mmol). The resulting solution was heated to 70° C. and stirred overnight. The reaction mixture was then cooled to room temperature and was quenched with H₂O (200 mL). The resulting mixture was extracted with EtOAc (3×1000 mL) and the combined organic layers were dried over Na₂SO₄, filtered, and concentrated under reduced pressure. The residue was purified by silica gel column chromatography (0→90% EtOAc/pet. ether) to afford the desired product (39.7 g, 85% yield). LCMS (ESI) m/z [M+H] calcd for C₄₀H₅₄N₆O₇S: 763.39; found: 763.3.

### Step 5: Synthesis of (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylic acid

To a solution of methyl (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylate (39.7 g, 52.0 mmol) in THE (400 mL) and H₂O (100 mL) at room temperature was added LiOH⋅H₂O (3.74 g, 156.2 mmol). The resulting mixture was stirred for 1.5 h and was then concentrated under reduced pressure. The residue was acidified to pH 6 with 1 M HCl and extracted with DCM (3×1000 mL). The combined organic layers were dried over Na₂SO₄, filtered, and concentrated under reduced pressure to afford the desired product (37.9 g, crude). LCMS (ESI) m/z [M+H] calcd for C₃₉H₅₂N₆O₇S: 749.37; found: 749.4.

### Step 6: Synthesis of tert-butyl ((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

To a solution of (S)-1-((S)-2-((tert-butoxycarbonyl)amino)-3-(4-(1-ethyl-3-(3-hydroxy-2,2-dimethylpropyl)-2-(2-((S)-1-methoxyethyl)pyridin-3-yl)-1H-indol-5-yl)thiazol-2-yl)propanoyl)hexahydropyridazine-3-carboxylic acid (37.9 g, 50.6 mmol), HOBt (34.19 g, 253.0 mmol) and DIPEA (264.4 mL, 1518 mmol) in DCM (4 L) at 0° C. was added EDCI (271.63 g, 1416.9 mmol). The resulting mixture was warmed to room temperature and stirred overnight. The reaction mixture was then quenched with H₂O and washed with 1 M HCl (4×1 L). The organic layer was separated and concentrated under reduced pressure. The residue was purified by silica gel column chromatography (0→70% EtOAc/pet. ether) to afford the desired product (30 g, 81% yield). LCMS (ESI) m/z [M+H] calcd for C₃₉H₅₀N₆O₆S: 731.36; found: 731.3.

### Intermediate F9. Synthesis of (6³S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione

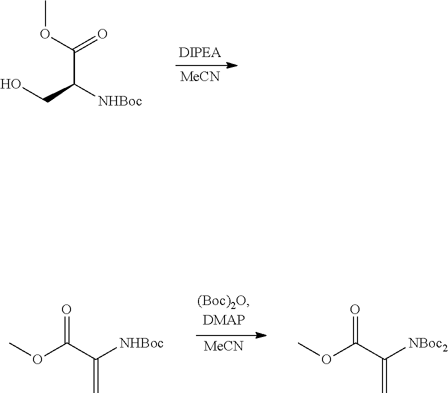

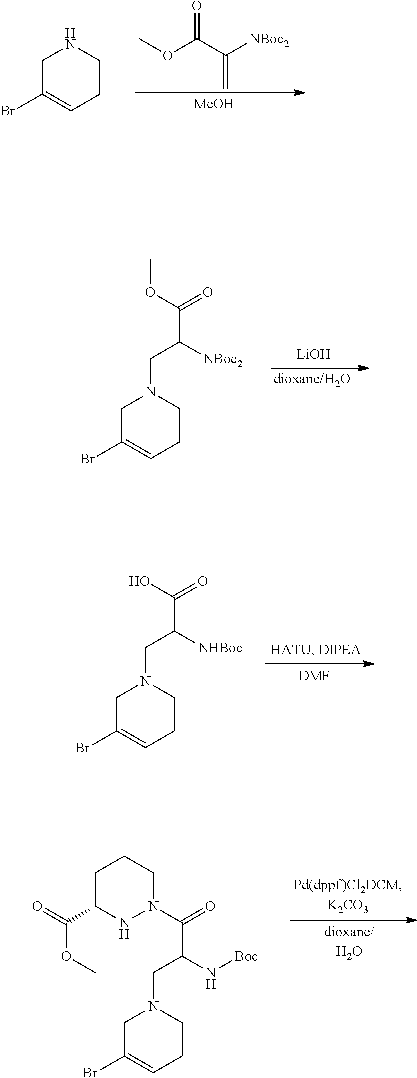

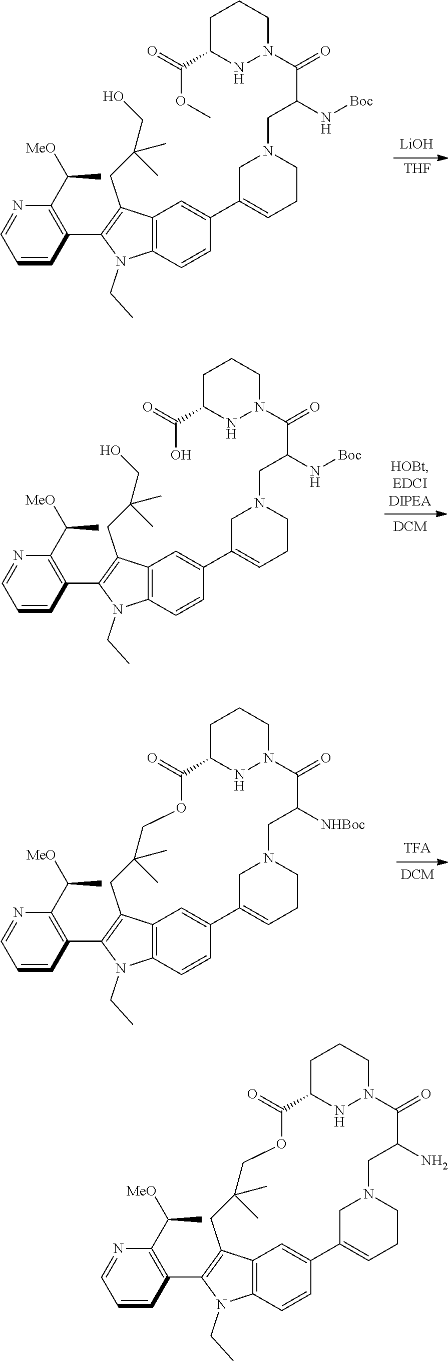

### Step 1: Synthesis of methyl 2-((tert-butoxycarbonyl)amino)acrylate

To a solution of methyl (tert-butoxycarbonyl)-L-serinate (10 g, 45 mmol) in anhydrous MeCN (150 mL), was added DIPEA (17 g, 137 mmol). The reaction mixture was stirred at 45° C. for 2 h to give the product in solution. LCMS (ESI) m/z [M+Na] calcd for C₉H₁₅NO₄ 201.1; found: 224.1.

### Step 2: Synthesis of methyl 2-(bis(tert-butoxycarbonyl)amino)acrylate

To a solution of methyl 2-((tert-butoxycarbonyl)amino)acrylate (12 g, 60 mmol) in anhydrous MeCN (150 mL) at 0° C., was added DMAP (13 g, 90 mmol) and (Boc)₂O (26 g, 120 mmol). The reaction was stirred for 6 h, then quenched with H₂O (100 mL) and extracted with DCM (3×200 mL). The combined organic layers were washed with brine (150 mL), dried over anhydrous Na₂SO₄, filtered and concentrated under reduced pressure. The residue was purified by silica gel column chromatography to give the product (12.5 g, 65% yield) as solid. LCMS (ESI) m/z [M+Na] calcd for C₁₄H₂₃NO₆ 301.2; found: 324.1.

### Step 3: Synthesis of methyl 2-(bis(tert-butoxycarbonyl)amino)-3-(5-bromo-3,6-dihydropyridin-1(2F)-yl)propanoate

To a mixture of 5-bromo-1,2,3,6-tetrahydropyridine (8.0 g, 49 mmol) in MeOH (120 mL) under an atmosphere of Ar was added methyl 2-{bis[(tert-butoxy)carbonyl]amino}prop-2-enoate (22 g, 74 mmol). The mixture was stirred for 16 h, then concentrated under reduced pressure and the residue was purified by silica gel column chromatography to give the product (12 g, 47% yield) as an oil. LCMS (ESI) m/z [M+H] calcd for C₁₉H₃₁BrN₂O₆ 462.1; found: 463.1.

### Step 4: Synthesis of 3-(5-bromo-3,6-dihydropyridin-1(2H)-yl)-2-((tert-butoxycarbonyl)amino)propanoic acid

To a mixture of methyl 2-(bis(tert-butoxycarbonyl)amino)-3-(5-bromo-3,6-dihydropyridin-1(2H)-yl)propanoate (14 g, 30 mmol) in dioxane (30 mL) and H₂O (12 mL) was added LiOH (3.6 g, 151 mmol). The mixture was heated to 35° C. and stirred for 12 h, then 1M HCl was added and the pH adjusted to ˜3-4. The mixture was extracted with DCM (2×300 mL) and the combined organic layers were dried over anhydrous Na₂SO₄ and filtered. The filtrate was concentrated under reduced pressure to give the product (10 g, 85% yield) as a solid. LCMS (ESI) m/z [M+H] calcd for C₁₃H₂₁BrN₂O₄ 348.1; found: 349.0.

### Step 5: Synthesis of methyl (3S)-1-(3-(5-bromo-3,6-dihydropyridin-1(2H)-yl)-2-((tert-butoxycarbonyl)amino)propanoyl)hexahydropyridazine-3-carboxylate

To a mixture of 3-(5-bromo-3,6-dihydropyridin-1(2H)-yl)-2-((tert-butoxycarbonyl)amino)propanoic acid (10 g, 30 mmol), DIPEA (12 g, 93 mmol) and methyl (3S)-1,2-diazinane-3-carboxylate (5.4 g, 37 mmol) in DMF (100 mL) at 0° C. under an atmosphere of Ar was added HATU (13 g, 34 mmol). The mixture was stirred at 0° C. for 2 h, then H₂O was added and the mixture extracted with EtOAc (2×300 mL). The combined organic layers were dried over anhydrous Na₂SO₄, filtered, the filtrate was concentrated under reduced pressure and the residue was purified by reverse phase chromatography to give the product (9.0 g, 55% yield) as a solid. LCMS (ESI) m/z [M+H] calcd for C₁₉H₃₁BrN₄O₅ 474.1; found: 475.1.

### Step 6: Synthesis of methyl (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylate

A mixture of methyl (3S)-1-(3-(5-bromo-3,6-dihydropyridin-1(2H)-yl)-2-((tert-butoxycarbonyl)amino)propanoyl)hexahydropyridazine-3-carboxylate (9.0 g, 18 mmol), K₂CO₃ (4.5 g, 32 mmol), Pd(dppf)Cl₂. DCM (1.4 g, 2 mmol), 3-(1-ethyl-2-{2-[(1S)-1-methoxyethyl]pyridin-3-yl}-5-(4,4,5,5-tetramethyl-1,3,2-dioxaborolan-2-yl)indol-3-yl)-2,2-dimethylpropan-1-ol (9.8 g, 20 mmol) in dioxane (90 mL) and H₂O (10 mL) under an atmosphere of Ar was heated to 75° C. and stirred for 2 h. H₂O was added and the mixture was extracted with EtOAc (3×200 mL). The combined organic layers were dried over Na₂SO₄, filtered, the filtrate was concentrated under reduced pressure and the residue was purified by silica gel column chromatography to give the product (4.0 g, 25% yield) as a solid. LCMS (ESI) m/z [M+H] calcd for C₄₂H₆₀N₆O₇ 760.5; found: 761.4.

### Step 7: Synthesis of (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylic acid

To a mixture of methyl (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylate (4.1 g, 5.0 mmol) in THE (35 mL) at 0° C. was added LiOH (0.60 g, 27 mmol). The mixture was stirred at 0° C. for 1.5 h, then 1M HCl added to adjust pH to ˜6-7 and the mixture was extracted with EtOAc (3×200 mL). The combined organic layers were dried over Na₂SO₄, filtered and the filtrate was concentrated under reduced pressure to give the product (3.6 g, 80% yield) as a solid. LCMS (ESI) m/z [M+H] calcd for C₄₁H₅₈N₆O₇ 746.4; found: 747.4.

### Step 8: Synthesis of tert-butyl ((63S)-1¹-ethyl-1²-(2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1 (5,3)-indola-6(1,3)-pyridazina-2(5,1)-pyridinacycloundecaphane-4-yl)carbamate

To a mixture of (3S)-1-(2-((tert-butoxycarbonyl)amino)-3-(5-(1-ethyl-3-(3-hydroxy-2,2-dimethylpropyl)-2-(2-((S)-1-methoxyethyl)pyridin-3-yl)-1H-indol-5-yl)-3,6-dihydropyridin-1(2H)-yl)propanoyl)hexahydropyridazine-3-carboxylic acid (3.6 g, 5.0 mmol) and DIPEA (24 g, 190 mmol) in DCM (700 mL) under an atmosphere of Ar was added EDCI⋅HCl (28 g, 140 mmol) and HOBt (6.5 g, 50 mmol). The mixture was heated to 30° C. and stirred for 16 h at 30° C., then concentrated under reduced pressure. The residue was diluted with EtOAc (200 mL) and washed with H₂O (2×200 mL), brine (200 mL), dried over Na₂SO₄ and filtered. The filtrate was concentrated under reduced pressure and the residue was purified by silica gel column chromatography to give the product (1.45 g, 40% yield) as a solid. LCMS (ESI) m/z [M+H] calcd for C₄₁H₅NO₆ 728.4; found: 729.4.

### Step 9: Synthesis of (6³S)-4-amino-1¹-ethyl-1²-(2-((S)-1-methoxyethyl)pyridin-3-yl)-10,10-dimethyl-2¹ 2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-5,7-dione

To a mixture of tert-butyl ((63S)-1¹-ethyl-1²-(2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1 (5,3)-indola-6(1,3)-pyridazina-2(5,1)- pyridinacycloundecaphane-4-yl)carbamate (130 mg, 0.20 mmol) in DCM (1.0 mL) at 0° C. was added TFA (0.3 mL). The mixture was warmed to room temperature and stirred for 2 h, then concentrated under reduced pressure to give the product, which was used directly in the next step directly without further purification. LCMS (ESI) m/z [M+H] calcd for C₃₆H₄₈N₆O₄ 628.4; found: 629.4.

### Intermediate F10. Synthesis of (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

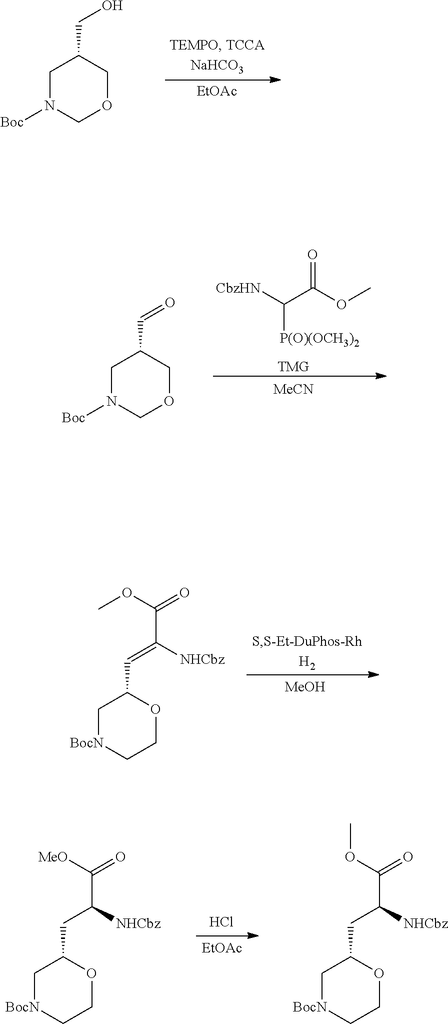

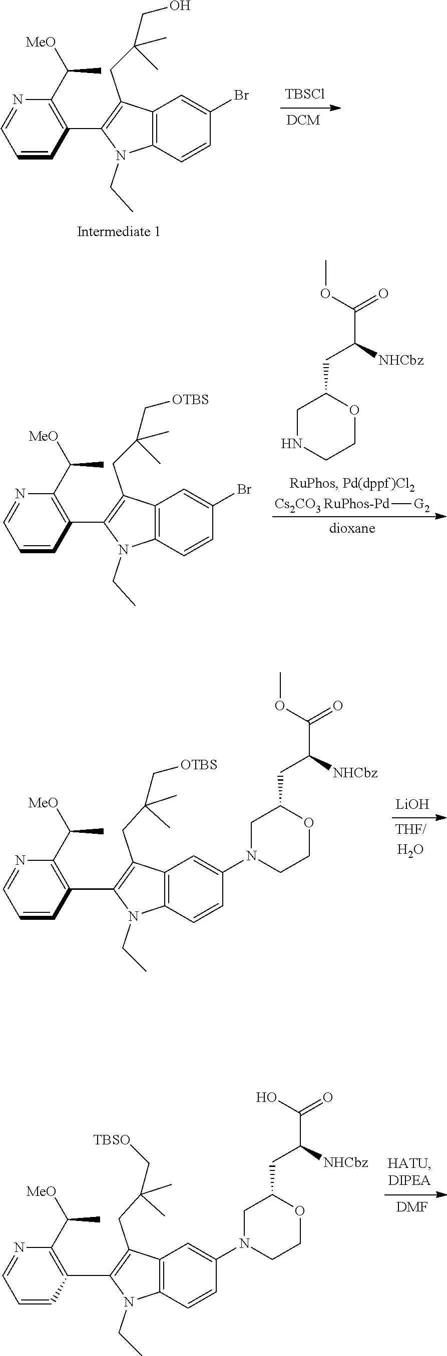

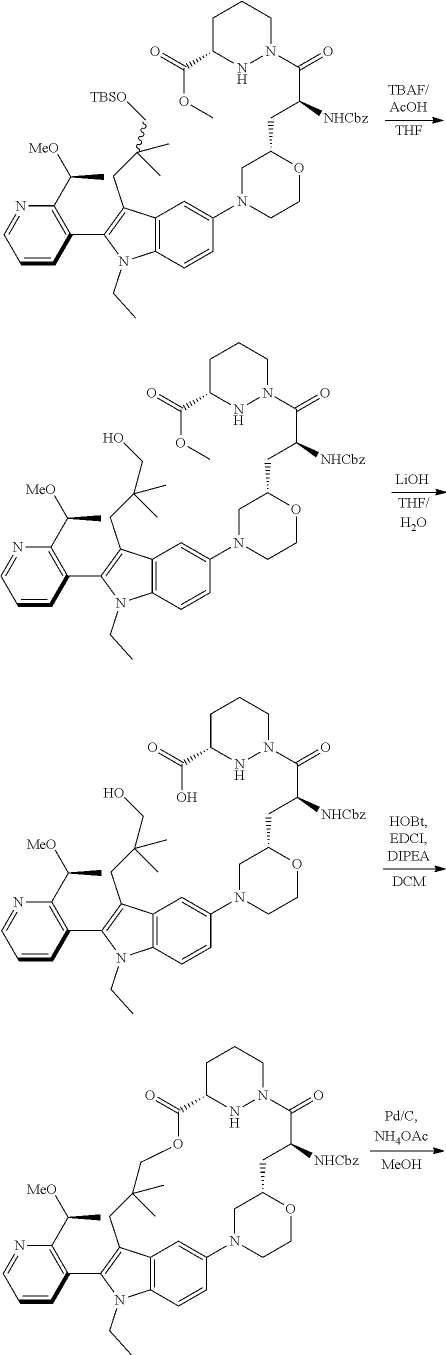

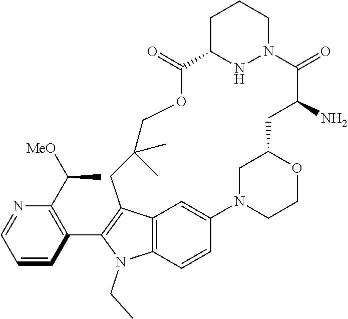

### Step 1: Synthesis of tert-butyl (2R)-2-formylmorpholin-4-yl formate

To a solution of tert-butyl (2R)-2-(hydroxymethyl)morpholin-4-yl formate (50 g, 230 mmol) in EtOAc (1 L) was added TEMPO (715 mg, 4.6 mmol) and NaHCO₃ (58 g, 690 mmol) at room temperature. The mixture was cooled to −50° C., then TCCA (56 g, 241 mmol) in EtOAc (100 mL) was added dropwise over 30 min. The reaction mixture was warmed to 5° C. for 2 h, then quenched with 10% Na₂S₂O₃ (200 mL) and stirred for 20 min. The resulting mixture was filtered and the organic phase was separated. The aqueous phase was extracted with EtOAc (2×100 mL). The combined organic layers were washed with H₂O (100 mL) and brine (100 mL), then dried over anhydrous Na₂SO₄. The organic layer was concentrated under reduced pressure to afford the product (50 g, crude) as an oil.

### Step 2: Synthesis of afford tert-butyl (S,Z)-2-(2-(((benzyloxy)carbonyl)amino)-3-methoxy-3-oxoprop-1-en-1-yl)morpholine-4-carboxylate

To a solution of tert-butyl (2R)-2-formylmorpholin-4-yl formate (49 g, 153 mmol) and methyl 2-{[(benzyloxy)carbonyl]amino}-2-(dimethoxyphosphoryl)acetate (60 g, 183 mmol) in MeCN (300 mL) was added tetramethylguanidine (35 g, 306 mmol) at 0-10° C. The reaction mixture was stirred at 10° C. for 30 min then warmed to room tempetature for 2 h. The reaction mixture was diluted with DCM (200 mL) and washed with 10% citric acid (200 mL) and 10% NaHCO₃ aq. (200 mL). The organic phase was concentrated under reduced pressure, and purified by silica gel column chromatography to afford the product (36 g, 90% yield) as solid. LCMS (ESI) m/z [M+Na] calcd for C₂₁H₂₈N₂O₄ 420.2; found: 443.1

### Step 3: Synthesis of tert-butyl (S)-2-((S)-2-(((benzyloxy)carbonyl)amino)-3-methoxy-3-oxopropyl)morpholine-4-carboxylate

To a solution of tert-butyl (S,Z)-2-(2-(((benzyloxy)carbonyl)amino)-3-methoxy-3-oxoprop-1-en-1-yl)morpholine-4-carboxylate (49 g, 0.12 mol) in MeOH (500 mL) was added (S,S)-Et-DUPHOS-Rh (500 mg, 0.7 mmol). The mixture was stirred at room temperature under an H₂ (60 psi) atmosphere for 48 h. The reaction was concentrated and purified by silica gel column chromatography to give the product (44 g, 90% yield) as solid. LCMS (ESI) m/z [M+Na] calcd for C₂₁H₃₀N₂O₇ 422.2; found: 445.2.

### Step 4: Synthesis of methyl (S)-2-(((benzyloxy)carbonyl)amino)-3-((S)-morpholin-2-yl)propanoate

To a stirred solution of tert-butyl (S)-2-((S)-2-(((benzyloxy)carbonyl)amino)-3-methoxy-3-oxopropyl)morpholine-4-carboxylate (2.2 g, 5.2 mmol) in EtOAc (2 mL) was added HCl/EtOAc (25 mL) at 15° C. The reaction was stirred at 15° C. for 2 h, then concentrated under reduced pressure to afford the product (1.51 g, 90% yield) as an oil. LCMS (ESI) m/z [M+H] calcd for C₁₆H₂₂N₂O₅ 322.1; found: 323.2.

### Step 5: Synthesis of (S)-5-bromo-3-(3-((tert-butyldimethylsilyl)oxy)-2,2-dimethylpropyl)-1-ethyl-2-(2-(1-methoxyethyl)pyridin-3-yl)-1H-indole

To a solution of 3-(5-bromo-1-ethyl-2-{2-[(1 S)-1-methoxyethyl]pyridin-3-yl}indol-3-yl)-2,2-dimethylpropan-1-ol (100 g, 0.22 mol) and imidazole (30.6 g, 0.45 mol) in DCM (800 mL) was added TBSCI (50.7 g, 0.34 mol) in DCM (200 mL) at 0° C. The reaction was stirred at room temperature for 2 h. The resulting solution was washed with H₂O (3×300 mL) and brine (2×200 mL), dried over anhydrous Na₂SO₄, filtered and concentrated under reduced pressure. The residue was purified with silica gel column chromatography to give the product (138 g, 90% yield) as a solid. LCMS (ESI) m/z [M+H] calcd for C₂₉H₄₃BrN₂O₂Si 558.2; found: 559.2.

### Step 6: Synthesis of methyl (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1 S)-1-methoxyethyl]pyridin-3-yl}indol-5-yl)morpholin-2-yl]propanoate

To a stirred solution of (S)-5-bromo-3-(3-((tert-butyldimethylsilyl)oxy)-2,2-dimethylpropyl)-1-ethyl-2-(2-(1-methoxyethyl)pyridin-3-yl)-1H-indole (50 g, 89.3 mmol) in dioxane (500 mL) was added methyl (2S)-2-{[(benzyloxy)carbonyl]amino}-3-[(2S)-morpholin-2-yl]propanoate (31.7 g, 98.2 mmol), RuPhos (16.7 g, 35.7 mmol), di-μ-chlorobis(2-amino-1,1-biphenyl-2-yl-C,N)dipalladium(II) (2.8 g, 4.4 mmol) and cesium carbonate (96 g, 295 mmol) followed by RuPhos-Pd-G2 (3.5 g, 4.4 mmol) at 105° C. under an N₂ atmosphere. The reaction mixture was stirred for 6 h at 105° C. under an N₂ atmosphere. The resulting mixture was filtered, and the filtrate was concentrated under reduced pressure. The residue was purified by prep-TLC chromatography to afford the product (55 g, 73% yield) as a solid. LCMS (ESI) m/z [M+H] calcd for C₄₅H₆₄N₄O₇Si 800.5; found: 801.5.

### Step 7: Synthesis of (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1 S)-1-methoxyethyl]pyridin-3-yl}indol-5-yl)morpholin-2-yl]propanoic acid

To a solution of methyl (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1S)-1-methoxyethyl]pyridin-3-yl}indol-5-yl)morpholin-2-yl]propanoate (10 g, 12 mmol) in THE (270 mL) was added LiOH (1.3 g, 31 mmol) in H₂O (45 mL) at room temperature. The reaction was stirred at room temperature for 2 h, then treated with 1 N HCl to adjust pH to 4˜5 at 0-5° C. The resulting mixture was extracted with EtOAc (2×50 mL). The combined organic layers were washed with brine and dried over anhydrous Na₂SO₄. The organic phase was then concentrated under reduced pressure to afford the product (9.5 g, 97% yield) as a solid. LCMS (ESI) m/z [M+H] calcd for C₄₄H₆₂N₄O₇Si 786.4; found: 787.4.

### Step 8: Synthesis of methyl (S)-1-((S)-2-(((benzyloxy)carbonyl)amino)-3-((S)-4-(3-(3-((tert-butyldimethylsilyl)oxy)-2,2-dimethylpropyl)-1-ethyl-2-(2-((S)-1-methoxyethyl)pyridin-3-yl)-1H-indol-5-yl)morpholin-2-yl)propanoyl)hexahydropyridazine-3-carboxylate

To a stirred solution of (2S)-2-{[(benzyloxy)carbonyl]amino}-3-[(2S)-4-(3-{3-[(tert-butyldimethylsilyl)oxy]-2,2-dimethylpropyl}-1-ethyl-2-{2-[(1 S)-1-methoxyethyl]pyridin-3-yl}indol-5-yl)morpholin-2-yl]propanoic acid (10 g, 12.7 mmol) in DMF (150 mL), was added methyl (S)-hexahydropyridazine-3-carboxylate (2 g, 14 mmol), then cooled to 0° C., DIPEA (32.8 g, 254 mmol) was added followed by HATU (9.7 g, 25.4 mmol) at 0-5° C. The reaction mixture was stirred at 0-5° C. for 1 h. The resulting mixture was diluted with EtOAc (500 mL) and H₂O (200 mL). The organic layer was separated and washed with H₂O (2×100 mL) and brine (100 mL), dried over anhydrous sodium sulfate. The solution was filtered and concentrated under reduced pressure, and the residue was purified by silica gel column chromatography to afford the product. LCMS (ESI) m/z [M+H] calcd for C₅₀H₇₂N₆O₈Si 912.5; found: 913.4.

### Step 9: Synthesis of methyl (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl)morpholin-2-yl)propanoyl)hexahydropyridazine-3-carboxylate

A solution of methyl (S)-1-((S)-2-(((benzyloxy)carbonyl)amino)-3-((S)-4-(3-(3-((tert-butyldimethylsilyl)oxy)-2,2-dimethylpropyl)-1-ethyl-2-(2-((S)-1-methoxyethyl)pyridin-3-yl)-1H-indol-5-yl)morpholin-2-yl)propanoyl)hexahydropyridazine-3-carboxylate (8.5 g, 9 mmol) in THE (8 mL) was added a mixture of tetrabutylammonium fluoride (1M in THF, 180 mL, 180 mmol) and AcOH (11 g, 200 mmol) at room temperature. The reaction mixture was stirred at 75° C. for 3 h. The resulting mixture was diluted with EtOAc (150 mL) and washed with H₂O (6×20 mL). The organic phase was concentrated under reduced pressure to give the product (7.4 g, 100% yield) as solid. LCMS (ESI) m/z [M+H] calcd for C₄₄H₅₈N₆O₈ 799.4; found: 798.4.

### Step 10: Synthesis of (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl)morpholin-2-yl)propanoyl)hexahydropyridazine-3-carboxylic acid

To a solution of methyl (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl)morpholin-2-yl)propanoyl)hexahydropyridazine-3-carboxylate (8 g, 10 mmol) in THE (200 mL) was added LiOH (600 mg, 25 mmol) in H₂O (30 mL). The reaction mixture was stirred at room temperature for 1 h, then treated with 1 N HCl to adjust pH to 4˜5 at 0-5° C., and extracted with EtOAc (2×500 mL). The organic phase was washed with brine and concentrated under reduced pressure to afford the product (8 g, crude) as a solid. LCMS (ESI) m/z [M+H] calcd for C₄₃H₅₆N₆O₈ 784.4; found: 785.4.

### Step 11: Synthesis of afford benzyl ((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)- pyridazinacycloundecaphane-4-yl)carbamate

To a stirred solution of (S)-1-((S)-2-(((benzyloxy)carbonyl)amino)-3-((S)-4-(1-ethyl-3-(3-hydroxy-2,2-dimethylpropyl)-2-(2-((S)-1-methoxyethyl)pyridin-3-yl)-1H-indol-5-yl)morpholin-2-yl)propanoyl)hexahydropyridazine-3-carboxylic acid (8 g, 10.2 mmol) and DIPEA (59 g, 459 mmol) in DCM (800 mL) was added EDCI (88 g, 458 mmol) and HOBt (27.6 g, 204 mmol) at room temperature under an argon atmosphere. The reaction mixture was stirred at room temperature for 16 h. The resulting mixture was concentrated under reduced pressure, and the residue was purified by silica gel column chromatography to afford the product (5 g, 66% yield) as a solid; LCMS (ESI) m/z [M+H] calcd for C₄₃H₅₄N₆O₇ 766.4; found: 767.4.

### Step 12: Synthesis of (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

To a solution of benzyl ((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1 (5,3)-indola-6(1,3)-pyridazinacycloundecaphane- 4-yl)carbamate (400 mg, 0.5 mmol) in MeOH (20 mL) was added Pd/C (200 mg) and ammonium acetate (834 mg, 16 mmol) at room temperature under an H₂ atmosphere and the mixture was stirred for 2 h. The resulting mixture was filtered and concentrated under reduced pressure. The residue was redissolved in DCM (20 mL) and washed with H₂O (5 mL×2), then concentrated under reduced pressure to afford the product (320 mg, 97% yield) as a solid. LCMS (ESI) m/z [M+H] calcd for C₃₅H₄₈N₆O₅ 632.4; found: 633.3.

### Intermediate F11. Synthesis of Tert-Butyl ((6³S,4S,Z)-11-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

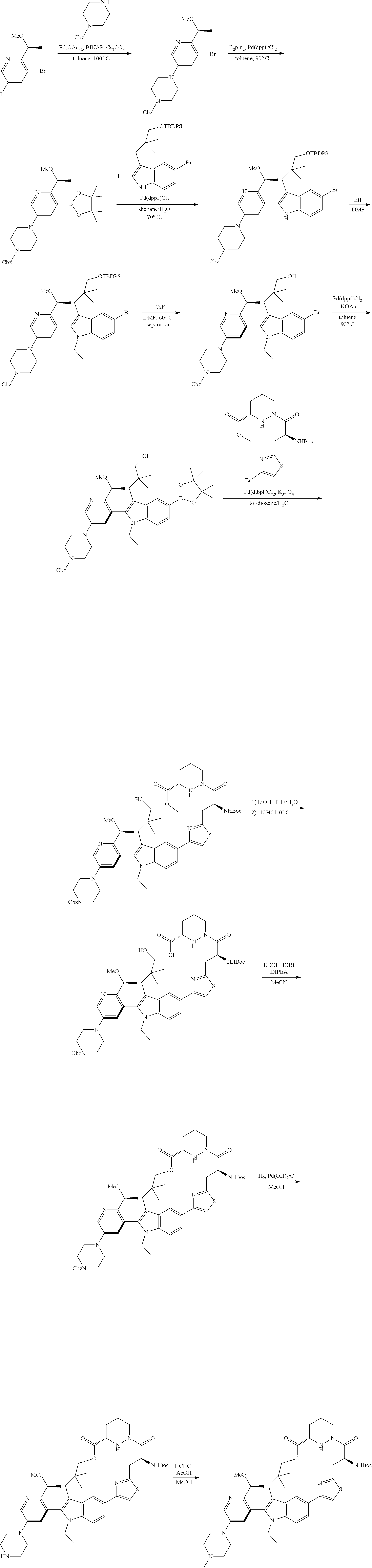

### Step 1: Synthesis of benzyl (S)-4-(5-bromo-6-(1-methoxyethyl)pyridin-3-yl)piperazine-1-carboxylate PP-3747, C₃

Into a 3L 3-necked round-bottom flask purged and maintained with an inert atmosphere of argon, was placed 3-bromo-5-iodo-2-[(1S)-1-methoxyethyl]pyridine (147 g, 429.8 mmol) benzyl piperazine-1-carboxylate (94.69 g, 429.8 mmol), Pd(OAc)₂ (4.83 g, 21.4 mmol), BINAP (5.35 g, 8.6 mmol), Cs₂CO₃ (350.14 g, 1074.6 mmol), toluene (1 L). The resulting solution was stirred overnight at 100° C. in an oil bath. The reaction mixture was then cooled to room temperature. The resulting mixture was concentrated under reduced pressure. The residue was purified by silica gel column chromatography (50% EtOAc/hexanes) to afford the product (135 g, 65% yield) as a solid. LCMS (ESI) m/z [M+H] calcd for C₂₀H₂₄BrN₃O₃ 433.1; found: 434.1.

### Step 2: Synthesis of benzyl (S)-4-(6-(1-methoxyethyl)-5-(4,4,5,5-tetramethyl-1,3,2-dioxaborolan-2-yl)pyridin-3-yl)piperazine-1-carboxylate

Into a 3-L 3-necked round-bottom flask purged and maintained with an inert atmosphere of argon, was placed benzyl 4-[5-bromo-6-[(1S)-1-methoxyethyl]pyridin-3-yl]piperazine-1-carboxylate (135 g, 310.8 mmol), bis(pinacolato)diboron (86.82 g, 341.9 mmol), Pd(dppf)Cl₂ (22.74 g, 31.0 mmol), KOAc (76.26 g, 777.5 mmol), toluene (1 L). The resulting solution was stirred for 2 days at 90° C. in an oil bath. The reaction mixture was cooled to room temperature. The resulting mixture was concentrated under reduced pressure. The residue was purified by neutral alumina column chromatography (30% EtOAC//hexane) to afford the product (167 g, crude) as a solid. LCMS (ESI) m/z [M+H] calcd for C₂₆H₃₆BN₃O₅ 481.3; found: 482.1.

### Step 3: Synthesis of benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl)piperazine-1-carboxylate

Into a 3-L 3-necked round-bottom flask purged and maintained with an inert atmosphere of argon, was placed (S)-4-(6-(1-methoxyethyl)-5-(4,4,5,5-tetramethyl-1,3,2-dioxaborolan-2-yl)pyridin-3-yl)piperazine-1-carboxylate (167 g, 346.9 mmol), 5-bromo-3-[3-[(tert-butyldiphenylsilyl)oxy]-2,2-dimethylpropyl]-2-iodo-1H-indole (224.27 g, 346.9 mmol), Pd(dppf)Cl₂ (25.38 g, 34.6 mmol), dioxane (600 mL), H₂O (200 mL), K₃PO₄ (184.09 g, 867.2 mmol), toluene (200 mL). The resulting solution was stirred overnight at 70° C. in an oil bath. The reaction mixture was then cooled to room temperature. The resulting mixture was concentrated under reduced pressure. The residue was purified by silica gel column chromatography (50% EtOAc/hexane) to afford the product (146 g, 48% yield) as a solid. LCMS (ESI) m/z [M+H] calcd for C₄₉H₅₇BrN₄O₄Si 872.3; found: 873.3.

### Step 4: Synthesis of benzyl (S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl)piperazine-1-carboxylate

To a stirred mixture of benzyl (S)-4-(5-(5-bromo-3-(3-((tert-butyldiphenylsilyl)oxy)-2,2-dimethylpropyl)-1H-indol-2-yl)-6-(1-methoxyethyl)pyridin-3-yl)piperazine-1-carboxylate (146 g, 167.0 mmol) and Cs₂CO₃ (163.28 g, 501.1 mmol) in DMF (1200 mL) was added ethyl iodide (52.11 g, 334.0 mmol) in portions at 0° C. under N₂ atmosphere. The final reaction mixture was stirred at room temperature for 12 h. The resulting mixture was diluted with EtOAc (1 L) and washed with brine (3×1.5L). The organic layers were dried over anhydrous Na₂SO₄. After filtration, the filtrate was concentrated under reduced pressure to give the product (143 g, crude) as a solid that was used directly for next step without further purification. LCMS (ESI) m/z [M+H] calcd for C₅₁H₆₁BrN₄O₄Si 900.4; found: 901.4.

### Step 5: Synthesis of benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate

To a stirred mixture of benzyl (S)-4-(5-(5-bromo-3-(3-((tert-butyldiphenylsilyl)oxy)-2,2-dimethylpropyl)-1-ethyl-1H-indol-2-yl)-6-(1-methoxyethyl)pyridin-3-yl)piperazine-1-carboxylate (143 g, 158.5 mmol) in DMF (1250 mL) was added CsF (72.24 g, 475.5 mmol). Then the reaction mixture was stirred at 60° C. for 2 days under an N₂ atmosphere. The resulting mixture was diluted with EtOAc (1 L) and washed with brine (3×1 L). Then the organic phase was concentrated under reduced pressure. The residue was purified by silica gel column chromatography (30% EtOAc/pet. ether) to afford two atropisomers A (38 g, 36% yield) and B (34 g, 34% yield) both as solids. LCMS (ESI) m/z [M+H] calcd for C₃₅H₄₃BrN₄O₄ 663.2; found: 662.2.

### Step 6: Synthesis of benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate

Into a 500-mL 3-necked round-bottom flask purged and maintained with an inert atmosphere of nitrogen, was placed benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate (14 g, 21.1 mmol), bis(pinacolato)diboron (5.89 g, 23.21 mmol), Pd(dppf)Cl₂ (1.54 g, 2.1 mmol), KOAc (5.18 g, 52.7 mmol), toluene (150 mL). The resulting solution was stirred for 5 h at 90° C. in an oil bath. The reaction mixture was cooled to room temperature then concentrated under reduced pressure. The residue was purified by silica gel column chromatography (30% EtOAc/pet. ether) to give the product (12 g, 76% yield) as a solid. LCMS (ESI) m/z [M+H] calcd for C₄₁H₅₅BN₄O₆ 710.4; found: 711.3.

### Step 7: Synthesis of methyl (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylate

Into a 250-mL round-bottom flask purged and maintained with an inert atmosphere of argon, was placed benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate (10.8 g, 15.2 mmol), methyl (3S)-1-[(2S)-3-(4-bromo-1,3-thiazol-2-yl)-2-[(tert-butoxycarbonyl)amino]propanoyl]-1,2-diazinane-3-carboxylate (7.98 g, 16.7 mmol), Pd(dtbpf)Cl₂ (0.99 g, 1.52 mmol), K₃PO₄ (8.06 g, 37.9 mmol), toluene (60 mL), dioxane (20 mL), H₂O (20 mL). The resulting solution was stirred for 3 h at 70° C. in an oil bath. The reaction mixture was cooled to room temperature. The resulting solution was extracted with EtOAc (2×50 mL) and concentrated under reduced pressure. The residue was purified by silica gel column chromatography (10% EtOAc/hexane). The solvent was removed under reduced pressure to give the product (8 g, 51% yield) as a solid. LCMS (ESI) m/z [M+H] calcd for C₅₂H₆₈N₈O₉S 980.5; found: 980.9.

### Step 8: Synthesis of (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylic acid

To a stirred mixture of methyl (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylate (12 g, 12.23 mmol) in THE (100 mL)/H₂O (100 mL) was added LiOH (2.45 g, 61.1 mmol) under an N₂ atmosphere and the resulting mixture was stirred for 2 h at room temperature. THE was removed under reduced pressure. The pH of the aqueous phase was acidified to 5 with 1N HCl at 0° C. The aqueous layer was extracted with DCM (3×100 mL). The organic phase was concentrated under reduced pressure to give the product (10 g, 85% yield) as a solid. LCMS (ESI) m/z [M+H] calcd for C₅₁H₆₆N₈O₉S 966.5; found: 967.0.

### Step 9: Synthesis of benzyl 4-(5-((6³S,4S,2)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl)piperazine-1-carboxylate

Into a 3-L round-bottom flask purged and maintained with an inert atmosphere of nitrogen, was placed (S)-1-((S)-3-(4-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)thiazol-2-yl)-2-((tert-butoxycarbonyl)amino)propanoyl)hexahydropyridazine-3-carboxylic acid (18 g, 18.61 mmol), MeCN (1.8 L), DIPEA (96.21 g, 744.4 mmol), EDCI (107.03 g, 558.3 mmol), and HOBt (25.15 g, 186.1 mmol). The resulting solution was stirred overnight at room temperature then concentrated under reduced pressure. The resulting solution was diluted with DCM (1 L) and washed with 1M HCl (3×1 L,) and H₂O (3×1 L). Then the organic layer was concentrated under reduced pressure and purified by silica gel column chromatography (50% EtOAc/hexane) to afford the product (10.4 g, 55% yield) as a solid. LCMS (ESI) m/z [M+H] calcd for C₅₁H₆₄N₈O₈S 948.5; found: 949.3.

### Step 10: Synthesis of tert-butyl ((63S,4S,2)-11-ethyl-12-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

Into a 250-mL round-bottom flask purged and maintained with an inert atmosphere of nitrogen, was placed benzyl 4-(5-((6³S,4S,2)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-1²-yl)-6-((S)-1-methoxyethyl)pyridin-3-yl)piperazine-1-carboxylate (10.40 g, 10.9 mmol), Pd(OH)₂/C (5 g, 46.9 mmol), MeOH (100 mL). The resulting solution was stirred for 3 h at room temperature under a 2 atm H₂ atmosphere. The solids were filtered out and the filter cake was washed with MeOH (3×100 mL). The combined organic phases were concentrated under reduced pressure to give the product (8.5 g, 90% yield) as a solid. LCMS (ESI) m/z [M+H] calcd for C₄₃H₅₈N₈O₆S 814.4; found: 815.3.

### Step 11: Synthesis of tert-butyl ((63S,4S,2)-1¹-ethyl-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

Into a 1000-mL round-bottom flask purged and maintained with an inert atmosphere of nitrogen, was placed tert-butyl ((6³S,4S,Z)-1¹-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (8.5 g, 10.4 mmol), MeOH (100 mL), AcOH (1.88 g, 31.2 mmol). The solution was stirred for 15 min and then HCHO (1.88 g, 23.15 mmol, 37% aqueous solution) and NaBH₃CN (788 mg, 12.5 mmol) were added at room temperature. The resulting solution was stirred for 3 h. The mixture was then quenched with H₂O (100 mL) and concentrated under reduced pressure to remove MeOH. The resulting solution was diluted with DCM (300 mL) and washed with H₂O (3×100 mL). The solution was concentrated under reduced pressure to afford the product (8.2 g, 90% yield) as a solid.

LCMS (ESI) m/z [M+H] calcd for C₄₄H₆₀N₈O₆S 828.4; found: 829.3.

### Intermediate F12: Synthesis of (6³S,4S,Z)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1H-8-oxa-2(4,2)-thiazola-1(5,3)-indola-6(1,3)-pyridazinacycloundecaphane-5,7-dione

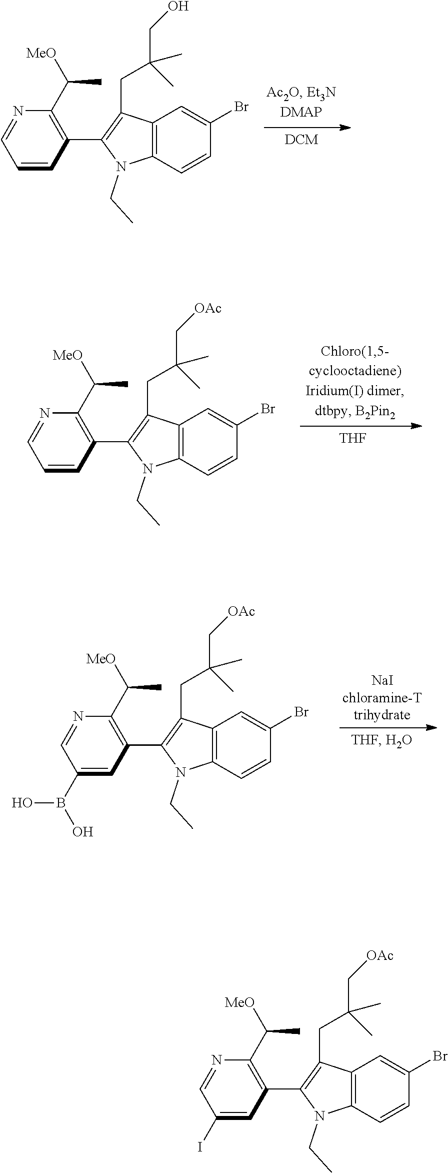

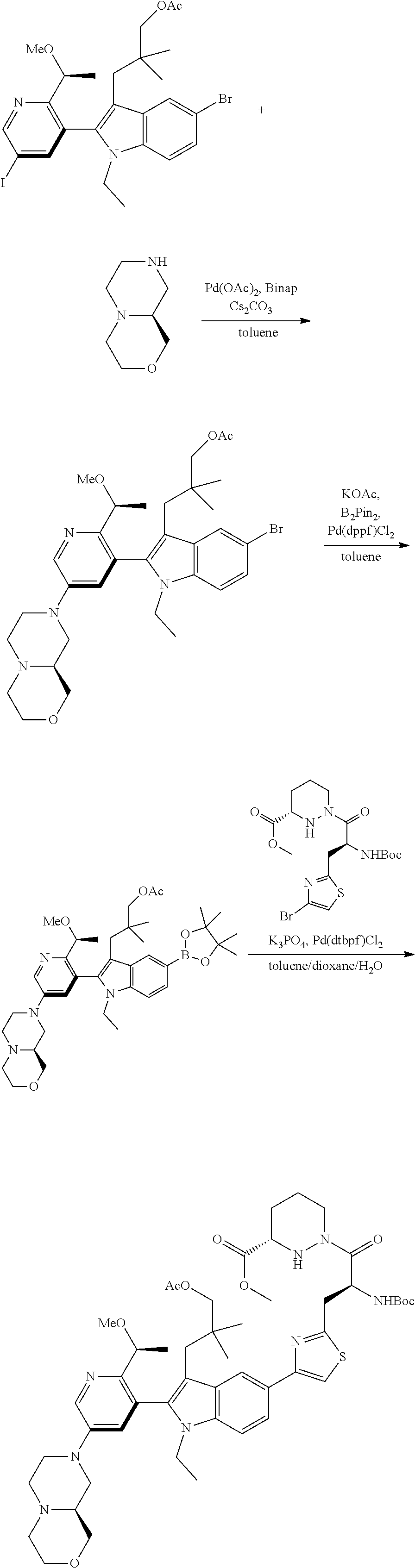

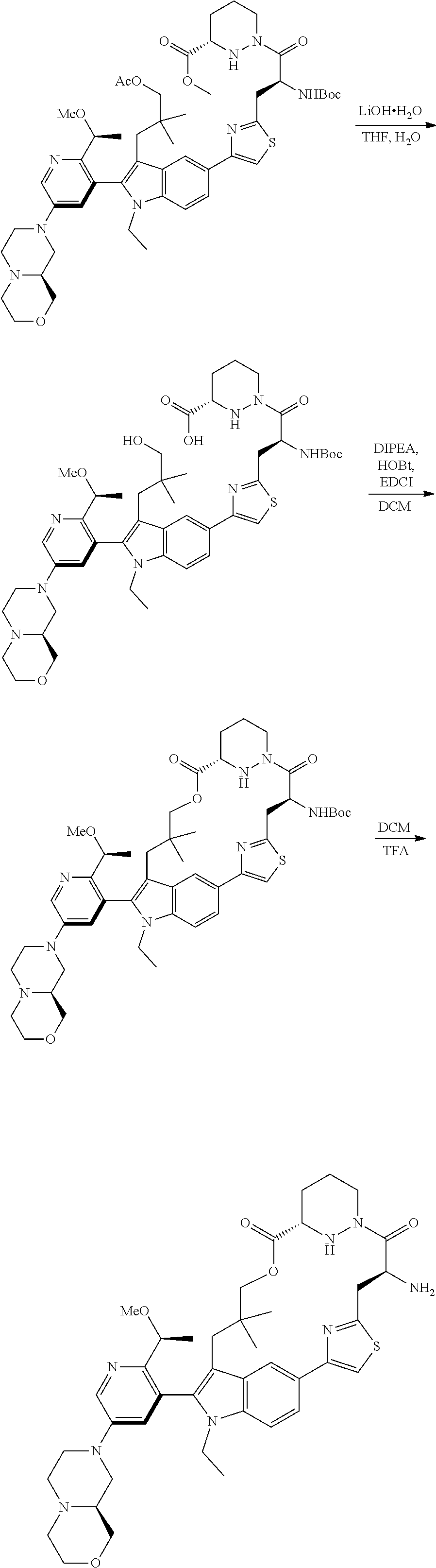

### Step 1: Synthesis of (S)-3-(5-bromo-1-ethyl-2-(2-(1-methoxyethyl)pyridin-3-yl)-1H-indol-3-yl)-2,2-dimethylpropyl acetate

To a stirred solution of (S)-3-(5-bromo-1-ethyl-2-(2-(1-methoxyethyl)pyridin-3-yl)-1H-indol-3-yl)-2,2-dimethylpropan-1-ol (100 g, 224.517 mmol) and Et₃N (45.44 g, 449.034 mmol) in DCM (1 L) was added DMAP (2.74 g, 22.452 mmol) and Ac₂O (27.50 g, 269.420 mmol) in portions at 0° C. under an argon atmosphere. The resulting mixture was stirred for 3 h at room temperature. The resulting mixture was concentrated under reduced pressure then diluted with EtOAc (1000 mL). The resulting mixture was washed with 1M HCl (500 mL) then washed with sat. NaHCO₃ (500 mL) and brine (500 mL) dried over anhydrous Na₂SO₄. After filtration, the filtrate was concentrated under reduced pressure. The residue was purified by trituration with pet. ether (500 mL) to afford the product (93.3 g, 85% yield) as a white solid.

LCMS (ESI) m/z [M+H] calcd for C₂₅H₃₁BrN₂O₃: 487.16; found: 489.2

### Step 2: Synthesis of (S)-(5-(3-(3-acetoxy-2,2-dimethylpropyl)-5-bromo-1-ethyl-1H-indol-2-yl)-6-(1-methoxyethyl)pyridin-3-yl)boronic acid

To a stirred solution of (S)-3-(5-bromo-1-ethyl-2-(2-(1-methoxyethyl)pyridin-3-yl)-1H-indol-3-yl)-2,2-dimethylpropyl acetate (93.3 g, 191.409 mmol) and B₂PIN₂ (72.91 g, 287.113 mmol) in THF (370 mL) was added dtbpy (7.71 g, 28.711 mmol) and chloro(1,5-cyclooctadiene)iridium(I) dimer (6.43 g, 9.570 mmol) in portions at room temperature under an argon atmosphere. The resulting mixture was stirred overnight at 75° C. The resulting mixture was concentrated under reduced pressure to afford the product (190 g, crude) as an oil. LCMS(ESI) m/z [M+H]; calcd for C₂₅H₃₂BBrN₂O₅: 531.17; found: 533.3

### Step 3: Synthesis of (S)-3-(5-bromo-1-ethyl-2-(5-iodo-2-(1-methoxyethyl)pyridin-3-yl)-1H-indol-3-yl)-2,2-dimethylpropyl acetate

To a stirred solution of (S)-(5-(3-(3-acetoxy-2,2-dimethylpropyl)-5-bromo-1-ethyl-1H-indol-2-yl)-6-(1-methoxyethyl)pyridin-3-yl)boronic acid (110 g, 207.059 mmol) and chloramine-T trihydrate (349.96 g, 1242.354 mmol) in THE (550 mL) was added a solution of NaI (186.22 g, 1242.354 mmol) in H₂O (225 mL) in portions at 0° C. under an air atmosphere. The resulting mixture was stirred overnight at 50° C. under an argon atmosphere. The resulting mixture was concentrated under reduced pressure then washed with CHCl₃ (500 mL). The resulting mixture was filtered, the filter cake was washed with CHCl₃ (3×250 mL). The filtrate was extracted with CHCl₃ (3×500 mL). The combined organic layers were washed with Na₂S₂O₃ (500 mL), washed with brine (2×200 mL) dried over anhydrous Na₂SO₄. After filtration, the filtrate was concentrated under reduced pressure. The residue was purified by silica gel column chromatography, (18% EtOAc/pet. ether) to afford the product (24 g, 18% yield) as a solid. LCMS(ESI) m/z [M+H]; calcd for C₂₅H₃₀BrIN₂O₃: 613.06; found: 614.7 Step 4: Synthesis of 3-(5-bromo-1-ethyl-2-(5-((S)-hexahydropyrazino[2,1-c][1,4]oxazin-8(1H)-yl)-2-((S)-1-methoxyethyl)pyridin-3-yl)-1H-indol-3-yl)-2,2-dimethylpropyl acetate

To a stirred solution of 3-(5-bromo-1-ethyl-2-{5-iodo-2-[(1 S)-1-methoxyethyl]pyridin-3-yl}indol-3-yl)-2,2-dimethylpropyl acetate (9 g, 14.674 mmol), (S)-octahydropyrazino[2,1-c][1,4]oxazine (2.469 g, 17.609 mmol), Cs₂CO₃ (11.953 g, 36.685 mmol,) and BINAP (456.9 mg, 0.734 mmol) in toluene (63 mL) was added Pd(OAc)₂ (329.44 mg, 1.467 mmol) at room temperature under an argon atmosphere. The resulting mixture was stirred for 6 h at 100° C. After filtration, the filter cake was washed with EtOAc (100 mL). The filtrate was concentrated under reduced pressure. The residue was purified by prep-TLC (8% MeOH/DCM) to afford the product (6.9 g, 75% yield) as a solid. LCMS (ESI) m/z [M+H] calcd for C₃₂H₄₃BrN₄O₄: 627.25; found: 627.4.

### Step 5: Synthesis of 3-(1-ethyl-2-(5-((S)-hexahydropyrazino[2,1-c][1,4]oxazin-8(1H)-yl)-2-((S)-1-methoxyethyl)pyridin-3-yl)-5-(4,4,5,5-tetramethyl-1,3,2-dioxaborolan-2-yl)-1H-indol-3-yl)-2,2-dimethylpropyl acetate

To a stirred solution of 3-(5-bromo-1-ethyl-2-(5-((S)-hexahydropyrazino[2,1-c][1,4]oxazin-8(1H)-yl)-2-((S)-1-methoxyethyl)pyridin-3-yl)-1H-indol-3-yl)-2,2-dimethylpropyl acetate (3.2 g, 5.115 mmol), KOAc (1.51 g, 15.345 mmol) and 4,4,5,5-tetramethyl-2-(tetramethyl-1,3,2-dioxaborolan-2-yl)-1,3,2-dioxaborolane (2.60 g, 10.230 mmol) in toluene (48 mL) was added Pd(dppf)Cl₂ (0.37 g, 0.512 mmol) in portions at room temperature under an argon atmosphere. The resulting mixture was stirred for 1.5 h at 90° C. The resulting mixture was filtered, the filter cake was washed with EtOAc (100 mL). The filtrate was concentrated under reduced pressure. The residue was purified by prep-TLC (8% MeOH/DCM) to afford the product (3.0 g, 88% yield) as a solid. LCMS (ESI) m/z [M+H] calcd for C₃₈H₅₅BN₄O₆: 675.43; found: 675.1

### Step 6: Synthesis of methyl (S)-1-((S)-3-(4-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)thiazol-2-yl)-2-((tert-butoxycarbonyl)amino)propanoyl)hexahydropyridazine-3-carboxylate

To a stirred mixture of 3-(1-ethyl-2-(5-((S)-hexahydropyrazino[2,1-c][1,4]oxazin-8(1H)-yl)-2-((S)-1-methoxyethyl)pyridin-3-yl)-5-(4,4,5,5-tetramethyl-1,3,2-dioxaborolan-2-yl)-1H-indol-3-yl)-2,2-dimethylpropyl acetate (5 g, 7.433 mmol) and K₃PO₄ (4.26 g, 20.067 mmol) in toluene (54 mL) were added dioxane (17.82 mL, 210.307 mmol) and H₂O (17.82 mL) at room temperature under an argon atmosphere. The resulting mixture was stirred for 2 h at 70° C. The resulting mixture was filtered, the filter cake was washed with EtOAc (100 mL). The filtrate was concentrated under reduced pressure. The resulting mixture was extracted with EtOAc (200 mL). The combined organic layers were washed with brine (100 mL), dried over anhydrous Na₂SO₄. After filtration, the filtrate was concentrated under reduced pressure. The residue was purified by prep-TLC (8% MeOH/DCM) to afford the product (4.6 g, 66% yield) as a solid. LCMS (ESI) m/z [M+H] calcd for C₄₉H₆₈N₈O₉S: 945.49; found: 945.7

### Step 7: Synthesis of (S)-1-((S)-2-((tert-butoxycarbonyl)amino)-3-(4-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)thiazol-2-yl)propanoyl)hexahydropyridazine-3-carboxylic acid

To a stirred solution of methyl (S)-1-((S)-3-(4-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)thiazol-2-yl)-2-((tert-butoxycarbonyl)amino)propanoyl)hexahydropyridazine-3-carboxylate (6 g, 6.361 mmol) in THE (43 mL) was added LiOH⋅H₂O (573.92 mg, 13.677 mmol) at 0° C. The resulting mixture was stirred for 16 h at room temperature. The mixture was acidified to pH 6 with HCl (aq.). The resulting mixture was extracted with EtOAc (3×100 mL). The combined organic layers were washed with brine, dried over anhydrous Na₂SO₄. After filtration, the filtrate was concentrated under reduced pressure. to afford the product (4 g, crude) as a solid. LCMS (ESI) m/z [M+H] calcd for C₄₅H₆₀N₈O₉S: 889.43: found: 889.7

### Step 8: Synthesis of tert-butyl ((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

To a stirred solution of (S)-1-((S)-2-((tert-butoxycarbonyl)amino)-3-(4-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)thiazol-2-yl)propanoyl)hexahydropyridazine-3-carboxylic acid (4 g, 4.51 mmol), HOBt (6.09 g, 45.09 mmol) and DIPEA (23.31 g, 180.36 mmol) in DCM (200 mL) was added EDCI (25.93 g, 135.27 mmol) in DCM (200 mL) dropwise at 0° C. under an argon atmosphere. The resulting mixture was stirred for 16 h at room temperature then concentrated under reduced pressure. The reaction was quenched with H₂O at 0° C. and extracted with EtOAc (500 mL). The combined organic layers were washed with brine, dried over anhydrous Na₂SO₄. After filtration, the filtrate was concentrated under reduced pressure. The residue was purified by prep-TLC (8% MeOH/DCM) to afford the product (2.0 g, 52% yield) as a solid. LCMS (ESI) m/z [M+H] calcd for C₄₆H₆₂N₈O₇S:870.4; found:871.8

### Step 9: Synthesis of (6³S,4S,Z)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

To a stirred solution of tert-butyl ((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (316 mg, 0.345 mmol) in DCM (3 mL,) was added TFA (1 mL) dropwise at 0° C. under an argon atmosphere. The resulting mixture was stirred for 2 h at room temperature. The mixture was basified to pH 8 with sat. aq. NaHCO₃. The resulting mixture was extracted with EtOAc (3×80 mL). The combined organic layers were washed with brine (3×40 mL), dried over anhydrous Na₂SO₄. After filtration, the filtrate was concentrated under reduced pressure. The crude product mixture was used in the next step directly without further purification. LCMS (ESI) m/z [M+H] calcd for C₄₁H₅₄N₈O₅S: 771.4; found: 771.6

### Intermediate F13: Synthesis of (6³S,4S,Z)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

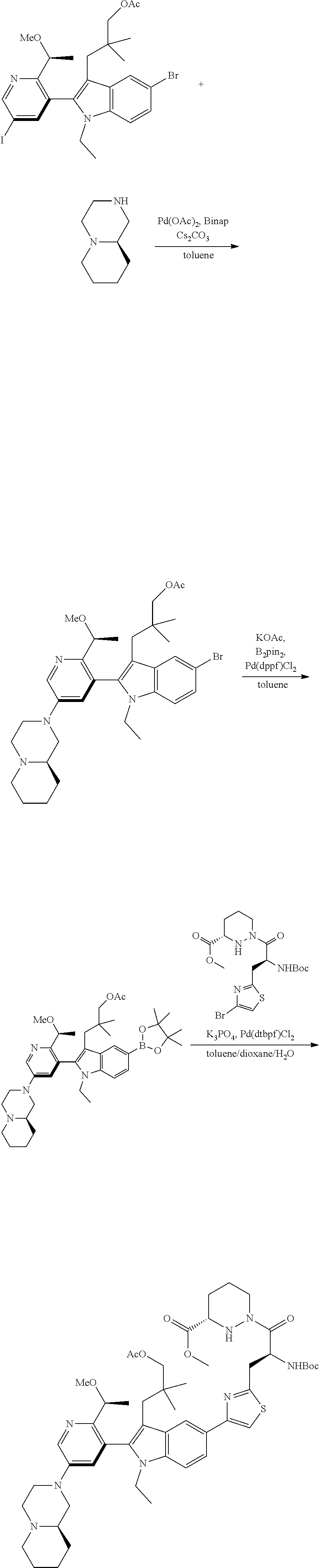

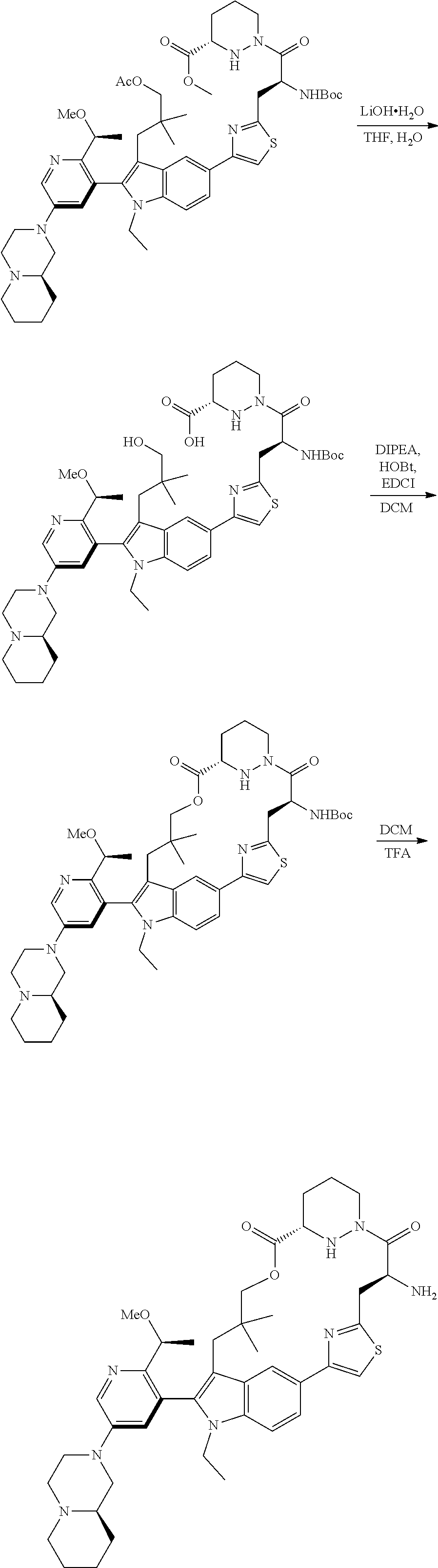

### Step 1: Synthesis of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate

To a stirred solution of 3-(5-bromo-1-ethyl-2-{5-iodo-2-[(1 S)-1-methoxyethyl]pyridin-3-yl}indol-3-yl)-2,2-dimethylpropyl acetate (9 g, 14.674 mmol), (R)-octahydro-2H-pyrido[1,2-a]pyrazine (2.469 g, 17.609 mmol), Cs₂CO₃ (11.9523 g, 36.685 mmol) and BINAP (456.85 mg, 0.734 mmol) in toluene (63 mL) was added Pd(OAc)₂ (329.44 mg, 1.467 mmol) in portions at room temperature under an argon atmosphere. The resulting mixture was stirred for 6 h at 100° C. then the mixture was filtered, the filter cake was washed with EtOAc (100 mL). The filtrate was concentrated under reduced pressure. The residue was purified by prep-TLC (8% MeOH/DCM) to afford the product (6 g, 65% yield) as a solid.

LCMS (ESI) m/z [M+H] calcd C₃₃H₄₅BrN₄O₃: 625.28; found: 627.4.

### Step 2: Synthesis of 3-(1-ethyl-2-(2-((S)-1-methoxyethyl)-5-((R)-octahydro-2H-pyrido[1,2-a]pyrazin-2-yl)pyridin-3-yl)-5-(4,4,5,5-tetramethyl-1,3,2-dioxaborolan-2-yl)-1H-indol-3-yl)-2,2-dimethylpropyl acetate

To a stirred solution of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (3.2 g, 5.115 mmol), KOAc (1.51 g, 15.345 mmol) and 4,4,5,5-tetramethyl-2-(tetramethyl-1,3,2-dioxaborolan-2-yl)-1,3,2-dioxaborolane (2.60 g, 10.230 mmol) in toluene (48 mL) was added Pd(dppf)Cl₂ (0.37 g, 0.512 mmol) in portions at room temperature under an argon atmosphere. The resulting mixture was stirred for 1.5 h at 90° C. The resulting mixture was filtered, the filter cake was washed with EtOAc (100 mL). The filtrate was concentrated under reduced pressure and purified by prep-TLC (8% MeOH/DCM) to afford the product (3.1 g, 81% yield) as a solid. LCMS (ESI) m/z [M+H] calcd C₃₉H₅₇BN₄O₅: 673.45; found: 673.4

### Step 3: Synthesis for methyl (S)-1-((S)-3-(4-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)thiazol-2-yl)-2-((tert-butoxycarbonyl)amino)propanoyl)hexahydropyridazine-3-carboxylate

To a stirred mixture of 3-(1-ethyl-2-(2-((S)-1-methoxyethyl)-5-((R)-octahydro-2H-pyrido[1,2-a]pyrazin-2-yl)pyridin-3-yl)-5-(4,4,5,5-tetramethyl-1,3,2-dioxaborolan-2-yl)-1H-indol-3-yl)-2,2-dimethylpropyl acetate (5 g, 7.433 mmol), methyl (S)-1-((S)-3-(4-bromothiazol-2-yl)-2-((tert-butoxycarbonyl)amino)propanoyl)hexahydropyridazine-3-carboxylate (3.89 g, 8.176 mmol) and K₃PO₄ (4.26 g, 20.067 mmol) in toluene (54 mL), dioxane (18 mL) and H₂O (18 mL) was added Pd(dtbpf)Cl₂ (969 mg, 1.486 mmol) at room temperature under an argon atmosphere. The resulting mixture was stirred for 2 h at 70° C. The mixture was filtered, the filter cake was washed with EtOAc (100 mL). The filtrate was concentrated under reduced pressure and the resulting mixture was extracted with EtOAc (200 mL). The combined organic layers were washed with brine (100 mL), dried over anhydrous Na₂SO₄. After filtration, the filtrate was concentrated under reduced pressure. The residue was purified by prep-TLC (8% MeOH/DCM) to afford the product (6.8 g, 83% yield) as a solid. LCMS (ESI) m/z [M+H] calcd C₅₀H₇₀N₈O₈S: 943.51; found: 943.4

### Step 4: Synthesis of (S)-1-((S)-2-((tert-butoxycarbonyl)amino)-3-(4-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)thiazol-2-yl)propanoyl)hexahydropyridazine-3-carboxylic acid

To a stirred solution of methyl (S)-1-((S)-3-(4-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)thiazol-2-yl)-2-((tert-butoxycarbonyl)amino)propanoyl)hexahydropyridazine-3-carboxylate (6 g, 6.361 mmol) in THE (43 mL) was added LiOH⋅H₂O (573.92 mg, 13.677 mmol) dropwise at 0° C. under an argon atmosphere. The resulting mixture was stirred for 16 h at room temperature. The mixture was acidified to pH 6 with HCl (aq.). The resulting mixture was extracted with EtOAc (3×150 mL). The combined organic layers were washed with brine, dried over anhydrous Na₂SO₄. After filtration, the filtrate was concentrated under reduced pressure to afford the product (4 g, crude) as a solid. LCMS (ESI) m/z [M+H] calcd C₄₇H₆₆N₈O₇S: 887.49; found: 887.6

### Step 5: Synthesis of tert-butyl ((6³S,4S,Z)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)-pyridazinacycloundecaphane-4-yl)carbamate

To a stirred solution of (S)-1-((S)-2-((tert-butoxycarbonyl)amino)-3-(4-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)thiazol-2-yl)propanoyl)hexahydropyridazine-3-carboxylic acid (4 g, 4.509 mmol), HOBt (6.09 g, 45.090 mmol) and DIPEA (23.31 g, 180.360 mmol) in DCM (200 mL) was added EDCI (25.93 g, 135.270 mmol) in DCM (200 mL) dropwise at 0° C. under an argon atmosphere. The resulting mixture was stirred for 16 h at room temperature. The resulting mixture was concentrated under reduced pressure and quenched with H₂O at 0° C. and extracted with EtOAc (500 mL). The combined organic layers were washed with brine, dried over anhydrous Na₂SO₄. After filtration, the filtrate was concentrated under reduced pressure. The residue was purified by prep-TLC (8% MeOH/DCM) to afford the product (2.0 g, 49% yield) as a solid. LCMS (ESI) m/z [M+H] calcd for C₄₇H₆₄N₈O₆S: 869.47; found: 869.8

### Step 6: Synthesis of (6³S,4S,Z)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

To a stirred solution of tert-butyl ((6³S,4S,Z)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)-pyridazinacycloundecaphane-4-yl)carbamate (900 mg, 1.035 mmol) in DCM (9 mL) was added TFA (3 mL) dropwise at 0° C. The resulting mixture was stirred for 2 h at room temperature. The mixture was basified to pH=8 with sat. aq. NaHCO₃. and extracted with EtOAc (3×100 mL). The combined organic layers were washed with brine (3×50 mL), dried over anhydrous Na₂SO₄. After filtration, the filtrate was concentrated under reduced pressure to afford the product (800 mg), which was used in the next step directly without further purification. LCMS (ESI) m/z [M+H] calcd for C₄₂H₅₆N₈O₄S: 769.42; found: 769.5

### Intermediate F14: Synthesis of (6³S,4S)-4-amino-1²-(5-((S)-hexahydropyrazino[2,1-c][1,4]oxazin-8(1H)-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

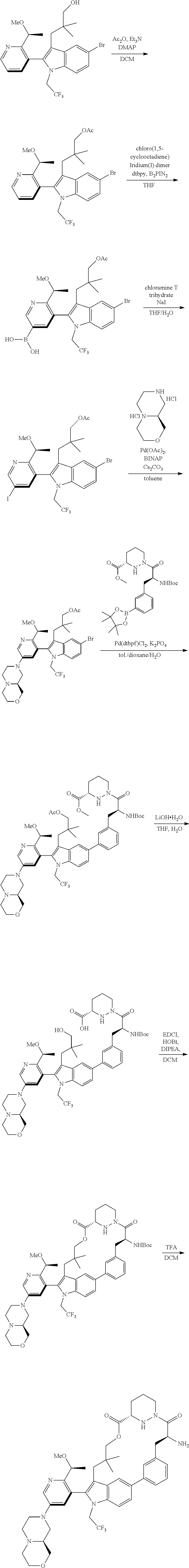

### Step 1: Synthesis of (S)-3-(5-bromo-2-(2-(1-methoxyethyl)pyridin-3-yl)-1-(2,2,2-trifluoroethyl)-1H-indol-3-yl)-2,2-dimethylpropyl acetate

To a stirred solution of (S)-3-(5-bromo-2-(2-(1-methoxyethyl)pyridin-3-yl)-1-(2,2,2-trifluoroethyl)-1H-indol-3-yl)-2,2-dimethylpropan-1-ol (60 g, 0.12 mol) and Et₃N (24.33 g, 0.24 mol) in DCM (600 mL) were added DMAP (1.46 g, 0.012 mol) and acetic anhydride (14.7 g, 144 mmol) dropwise at 0° C. under an argon atmosphere. The resulting mixture was stirred for 2 h at room temperature. The resulting mixture was concentrated under reduced pressure and washed with of HCl (500 mL). The resulting mixture was washed with of sat. aq. NaHCO₃ (500 mL). The combined organic layers were washed with brine (500 mL), dried over anhydrous Na₂SO₄. After filtration, the filtrate was concentrated under reduced pressure to afford the product (59.6 g, 92% yield) as a n oil. LCMS (ESI) m/z [M+H] calcd C₂₅H₂₈BrF₃N₂O₃: 541.13; found: 543.2

### Step 2: Synthesis of (S)-(5-(3-(3-acetoxy-2,2-dimethylpropyl)-5-bromo-1-(2,2,2-trifluoroethyl)-1H-indol-2-yl)-6-(1-methoxyethyl)pyridin-3-yl)boronic acid

To a stirred mixture of (S)-3-(5-bromo-2-(2-(1-methoxyethyl)pyridin-3-yl)-1-(2,2,2-trifluoroethyl)-1H-indol-3-yl)-2,2-dimethylpropyl acetate (55.1 g, 101.771 mmol) and 4,4,5,5-tetramethyl-2-(tetramethyl-1,3,2-dioxaborolan-2-yl)-1,3,2-dioxaborolane (38.77 g, 152.656 mmol) in THE (40 mL) were added dtbpy (4.10 g, 15.266 mmol) and Chloro(1,5-cyclooctadiene)iridium(I) dimer (3.42 g, 5.089 mmol) in portions at room temperature under an argon atmosphere. The resulting mixture was stirred for 5 h at 75° C. The resulting mixture was concentrated under reduced pressure to afford the product (102.4 g, crude) as an oil. LCMS (ESI) m/z [M+H] calcd C₂₅H₂₉BBrF₃N₂O₅: 585.14; found: 585.2

### Step 3: Synthesis of (S)-3-(5-bromo-2-(5-iodo-2-(1-methoxyethyl)pyridin-3-yl)-1-(2,2,2-trifluoroethyl)-1H-indol-3-yl)-2,2-dimethylpropyl acetate

To a stirred solution of (S)-(5-(3-(3-acetoxy-2,2-dimethylpropyl)-5-bromo-1-(2,2,2-trifluoroethyl)-1H-indol-2-yl)-6-(1-methoxyethyl)pyridin-3-yl)boronic acid (51.2 g, 87.487 mmol) and sodium chloro[(4-methylbenzene)sulfonyl]azanide (197 g, 699.896 mmol) in THE (258 mL) was added NaI (104.91 g, 699.896 mmol) in H₂O (129 mL) dropwise at 0° C. under an argon atmosphere. The resulting mixture was stirred for 16 h at 55° C. The resulting mixture was concentrated under reduced pressure and extracted with CH₃Cl (2×200 mL). The combined organic layers were washed with brine (2×20 mL), dried over anhydrous Na₂SO₄. After filtration, the filtrate was concentrated under reduced pressure. The residue was purified by silica gel column chromatography (20% EtOAc/pet. ether) to afford the product (15.3 g, 26% yield) as a solid. LCMS (ESI) m/z [M+H] calcd C₃₂H₄₀BrF₃N₄O₄: 666.0; found: 667.3

### Step 4: Synthesis of methyl (S)-1-((S)-3-(3-(3-(3-acetoxy-2,2-dimethylpropyl)-2-(5-((S)-hexahydropyrazino[2,1-c][1,4]oxazin-8(1H)-yl)-2-((S)-1-methoxyethyl)pyridin-3-yl)-1-(2,2,2-trifluoroethyl)-1H-indol-5-yl)phenyl)-2-((tert-butoxycarbonyl)amino)propanoyl)hexahydropyridazine-3-carboxylate

To a stirred mixture of (S)-3-(5-bromo-2-(5-iodo-2-(1-methoxyethyl)pyridin-3-yl)-1-(2,2,2-trifluoroethyl)-1H-indol-3-yl)-2,2-dimethylpropyl acetate (2.70 g, 4.046 mmol) and (S)-octahydropyrazino[2,1-c][1,4]oxazine dihydrochloride (1.044 g, 4.855 mmol) in toluene(18.9 mL) was added Cs₂CO₃ (5932.38 mg, 18.207 mmol) and BINAP (125.97 mg, 0.202 mmol) in portions at room temperature under an argon atmosphere. To the above mixture was added Pd(OAc)₂ (90.84 mg, 0.405 mmol) in portions. The resulting mixture was stirred for additional 16 h at 90° C. The mixture was cooled to room temperature then filtered, the filter cake was washed with EtOAc (2×20 mL). The filtrate was concentrated under reduced pressure. The residue was purified by silica gel column chromatography (10% MeOH/DCM) to afford the product (2.3 g, 83% yield) as a solid. LCMS (ESI) m/z [M+H] calcd C₃₂H₄₀BrF₃N₄O₄: 681.23; found: 681.4

### Step 5: Synthesis of methyl (S)-1-((S)-3-(3-(3-(3-acetoxy-2,2-dimethylpropyl)-2-(5-((S)-hexahydropyrazino[2,1-c][1,4]oxazin-8(1H)-yl)-2-((S)-1-methoxyethyl)pyridin-3-yl)-1-(2,2,2-trifluoroethyl)-1H-indol-5-yl)phenyl)-2-((tert-butoxycarbonyl)amino)propanoyl)hexahydropyridazine-3-carboxylate

Into a 250 mL 3-necked round-bottom flask was added methyl (S)-1-((S)-3-(3-(3-(3-acetoxy-2,2-dimethylpropyl)-2-(5-((S)-hexahydropyrazino[2,1-c][1,4]oxazin-8(1H)-yl)-2-((S)-1-methoxyethyl)pyridin-3-yl)-1-(2,2,2-trifluoroethyl)-1H-indol-5-yl)phenyl)-2-((tert-butoxycarbonyl)amino)propanoyl)hexahydropyridazine-3-carboxylate (2.33 g, 4.512 mmol) and K₃PO₄ (1.59 g, 7.490 mmol) at room temperature under an air atmosphere. To a stirred mixture of H₂O (8.20 mL), and dioxane (8.20 mL) in toluene was added Pd(dtbpf)Cl₂ (0.29 g, 0.451 mmol) in portions at room temperature. The resulting mixture was stirred for 3 h at 65° C. The resulting mixture was filtered, the filter cake was washed with EtOAc (2×100 mL). The filtrate was concentrated under reduced pressure. The resulting mixture was extracted with EtOAc (2×100 mL). The combined organic layers were washed with brine (2×150 mL), dried over anhydrous Na₂SO₄. After filtration, the filtrate was concentrated under reduced pressure. The residue was purified by silica gel column chromatography (3→4% MeOH/DCM) to afford the product (2.7 g, 90% yield) as a solid. LCMS (ESI) m/z [M+H] calcd C₅₂H₆₈F₃N₇O₉: 991.5; found: 992.7

### Step 6: Synthesis of (S)-1-((S)-2-((tert-butoxycarbonyl)amino)-3-(3-(2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1-(2,2,2-trifluoroethyl)-1H-indol-5-yl)phenyl)propanoyl)hexahydropyridazine-3-carboxylic acid

Into a 100 mL 3-necked round-bottom flask were added methyl (S)-1-((S)-3-(3-(3-(3-acetoxy-2,2-dimethylpropyl)-2-(5-((S)-hexahydropyrazino[2,1-c][1,4]oxazin-8(1H)-yl)-2-((S)-1-methoxyethyl)pyridin-3-yl)-1-(2,2,2-trifluoroethyl)-1H-indol-5-yl)phenyl)-2-((tert-butoxycarbonyl)amino)propanoyl)hexahydropyridazine-3-carboxylate (3 g, 3.024 mmol) and THE (30 mL) at room temperature. Followed by LiOH (0.30 g, 12.701 mmol) in H₂O (12.7 mL) in portions at 0° C. The resulting mixture was stirred for 16 h at room temperature. The mixture was acidified to pH 5 with 1 N HCl. The resulting mixture was extracted with EtOAc (2×100 mL). The combined organic layers were washed with brine (2×100 mL), dried over anhydrous Na₂SO₄. After filtration, the filtrate was concentrated under reduced pressure to afford the product (2.7 g, crude) as a solid. LCMS (ESI) m/z [M+H] calcd for C₄₉H₆₄F₃N₇O₈: 936.48; found: 936.7

### Step 7: synthesis of tert-butyl ((63S,4S)-12-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

Into a 2 L 3-necked round-bottom flask were added (S)-1-((S)-2-((tert-butoxycarbonyl)amino)-3-(3-(2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1-(2,2,2-trifluoroethyl)-1H-indol-5-yl)phenyl)propanoyl)hexahydropyridazine-3-carboxylic acid (3.12 g, 3.333 mmol) and DCM (624 mL) at room temperature. To the above mixture was added DIPEA (17.23 g, 133.320 mmol) and HOBt (4.50 g, 33.330 mmol) in portions at 0° C. The resulting mixture was stirred for additional 30 min. To the above mixture was added EDCI (19.17 g, 99.990 mmol) in portions over 16 h at room temperature. The resulting mixture was concentrated under reduced pressure. The reaction was quenched with H₂O at 0° C. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography (3→4% MeOH/DCM) to afford the product (3 g, 98% yield) as a solid. LCMS (ESI) m/z [M+H] calcd C₄₉H₆₂F₃N₇O₇: 918.47; found: 918.8

### Step 8: Synthesis of (6³S,4S)-4-amino-1²-(5-((S)-hexahydropyrazino[2,1-c][1,4]oxazin-8(1H)-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a stirred solution of tert-butyl ((63S,4S)-12-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (930 mg, 1.013 mmol) in DCM (15 mL) was added TFA (5 mL, 67.315 mmol) dissolved in DCM (5 mL) dropwise at 0° C. under an argon atmosphere. The resulting mixture was stirred for 2 h at 0° C. The residue was basified to pH 8 with sat. aq. NaHCO₃. The resulting mixture was extracted with DCM, the combined organic layers were washed with brine, dried over anhydrous Na₂SO₄. After filtration, the filtrate was concentrated under reduced pressure to afford the product (880 mg, crude) as a solid. LCMS (ESI) m/z [M+H] calcd for C₄₄H₅₄F₃N₇O₅: 818.42; found: 818.6

### Intermediate F15: Synthesis of (6³S,4S)-4-amino-12-(2-((S)-1-methoxyethyl)-5-((R)-octahydro-2H-pyrido[1,2-a]pyrazin-2-yl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

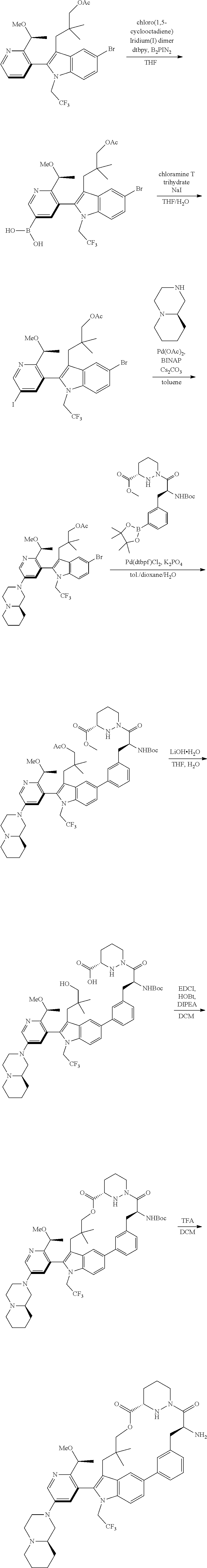

### Step 1: Synthesis of (S)-(5-(3-(3-acetoxy-2,2-dimethylpropyl)-5-bromo-1-(2,2,2-trifluoroethyl)-1H-indol-2-yl)-6-(1-methoxyethyl)pyridin-3-yl)boronic acid

Into a 100 mL 3-necked round-bottom flask were added 3-(5-bromo-2-{2-[(1S)-1-methoxyethyl]pyridin-3-yl}-1-(2,2,2-trifluoroethyl)indol-3-yl)-2,2-dimethylpropyl acetate (10 g, 18.470 mmol), 4,4,5,5-tetramethyl-2-(tetramethyl-1,3,2-dioxaborolan-2-yl)-1,3,2-dioxaborolane (8.44 g, 33.25 mmol) and dtbpy (0.89 g, 3.325 mmol) at room temperature. To the above mixture was added chloro(1,5-cyclooctadiene)iridium(I) dimer (0.74 g, 1.108 mmol) and THE (40 mL). The resulting mixture was stirred for additional 16 h at 80° C. The resulting mixture was concentrated under reduced pressure. The crude product was used in the next step directly without further purification. LCMS (ESI) m/z [M+H] calcd for C₂₅H₂₉BBrF₃N₂O₅: 585.14; found: 585.0

### Step 2: Synthesis of (S)-3-(5-bromo-2-(5-iodo-2-(1-methoxyethyl)pyridin-3-yl)-1-(2,2,2-trifluoroethyl)-1H-indol-3-yl)-2,2-dimethylpropyl acetate

To a stirred solution of (S)-(5-(3-(3-acetoxy-2,2-dimethylpropyl)-5-bromo-1-(2,2,2-trifluoroethyl)-1H-indol-2-yl)-6-(1-methoxyethyl)pyridin-3-yl)boronic acid (17.9 g, 30.586 mmol) in THE (89.5 mL,) were added sodium chloro[(4-methylbenzene)sulfonyl]azanide (68.93 g, 244.688 mmol) and NaI (36.68 g, 244.688 mmol) in H₂O (44.75 mL) dropwise at 0° C. under a nitrogen atmosphere. The resulting mixture was stirred for additional 20 min at room temperature then heated to 50° C. for 16 h. The resulting mixture was concentrated under reduced pressure and washed with CHCl₃ (300 mL). After filtration, the filter cake was washed with CHCl₃ (3×100 mL). The filtrate was extracted with CHCl₃ (3×200 mL). The combined organic layers were washed with Na₂S₂O₃ (300 mL), and brine (2×150 mL), dried over anhydrous Na₂SO₄. After filtration, the filtrate was concentrated under reduced pressure. The residue was purified by silica gel column chromatography (16% EtOAc/pet. ether) to afford the product (6.6 g, 32% yield) as a solid. LCMS (ESI) m/z [M+H] calcd for C₂₅H₂₇BrF₃IN₂O₃: 667.03; found: 668.7

### Step 3: Synthesis of 3-(5-bromo-2-(2-((S)-1-methoxyethyl)-5-((R)-octahydro-2H-pyrido[1,2-a]pyrazin-2-yl)pyridin-3-yl)-1-(2,2,2-trifluoroethyl)-1H-indol-3-yl)-2,2-dimethylpropyl acetate

To a stirred mixture of (S)-3-(5-bromo-2-(5-iodo-2-(1-methoxyethyl)pyridin-3-yl)-1-(2,2,2-trifluoroethyl)-1H-indol-3-yl)-2,2-dimethylpropyl acetate (1.4 g, 2.098 mmol) and (R)-octahydro-2H-pyrido[1,2-a]pyrazine (353.04 mg, 2.518 mmol) in toluene (10 mL) was added Cs₂CO₃ (3076.05 mg, 9.441 mmol), BINAP (65.32 mg, 0.105 mmol) and Pd(OAc)₂ (47.10 mg, 0.210 mmol). The resulting mixture was stirred overnight at 90° C. under an argon atmosphere. The reaction was quenched with H₂O (100 mL). The resulting mixture was extracted with DCM (3×100 mL). The combined organic layers were washed with H₂O (3×100 mL), dried over anhydrous Na₂SO₄. After filtration, the filtrate was concentrated under reduced pressure. The residue was purified by silica gel column chromatography (5% MeOH/DCM) to afford the product (1 g, 49% yield) as an oil. LCMS (ESI) m/z [M+H] calcd for C₃₃H₄₂BrF₃N₄O₃: 679.25; found: 679.5

### Step 4: Synthesis of methyl (S)-1-((S)-3-(3-(3-(3-acetoxy-2,2-dimethylpropyl)-2-(2-((S)-1-methoxyethyl)-5-((R)-octahydro-2H-pyrido[1,2-a]pyrazin-2-yl)pyridin-3-yl)-1-(2,2,2-trifluoroethyl)-1H-indol-5-yl)phenyl)-2-((tert-butoxycarbonyl)amino)propanoyl)hexahydropyridazine-3-carboxylate

To a stirred mixture of 3-(5-bromo-2-(2-((S)-1-methoxyethyl)-5-((R)-octahydro-2H-pyrido[1,2-a]pyrazin-2-yl)pyridin-3-yl)-1-(2,2,2-trifluoroethyl)-1H-indol-3-yl)-2,2-dimethylpropyl acetate (1 g, 1.471 mmol) and methyl (S)-1-((S)-2-((tert-butoxycarbonyl)amino)-3-(3-(4,4,5,5-tetramethyl-1,3,2-dioxaborolan-2-yl)phenyl)propanoyl)hexahydropyridazine-3-carboxylate (913.62 mg, 1.765 mmol) in toluene (9 mL) were added dioxane (6 mL), H₂O (3 mL), K₃PO₄ (780.82 mg, 3.678 mmol) and Pd(dtbpf)Cl₂ (95.90 mg, 0.147 mmol), the resulting mixture was stirred for 2 h at 70° C. under a nitrogen atmosphere. The mixture was basified to pH 8 with sat. aq. NaHCO₃. The resulting mixture was extracted with DCM (3×30 mL). The combined organic layers were washed with H₂O (3×30 mL), dried over anhydrous Na₂SO₄. After filtration, the filtrate was concentrated under reduced pressure. The residue was purified by silica gel column chromatography (20% EtOAc/pet. ether) to afford the product (1.2 g, 74% yield) as a solid. LCMS (ESI) m/z [M+H] calcd for C₅₃H₇₀F₃N₇O₈: 990.53; found: 990.8

### Step 5: Synthesis of (S)-1-((S)-2-((tert-butoxycarbonyl)amino)-3-(3-(3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1-(2,2,2-trifluoroethyl)-1H-indol-5-yl)phenyl)propanoyl)hexahydropyridazine-3-carboxylic acid

To a stirred mixture of methyl (S)-1-((S)-3-(3-(3-(3-acetoxy-2,2-dimethylpropyl)-2-(2-((S)-1-methoxyethyl)-5-((R)-octahydro-2H-pyrido[1,2-a]pyrazin-2-yl)pyridin-3-yl)-1-(2,2,2-trifluoroethyl)-1H-indol-5-yl)phenyl)-2-((tert-butoxycarbonyl)amino)propanoyl)hexahydropyridazine-3-carboxylate (1.2 g, 1.212 mmol) and LiOH (252 mg, 10.523 mmol) in THE (6 mL) was added H₂O (6 mL) in portions at 0° C. The resulting mixture was stirred overnight at 0° C. The mixture was acidified to pH 7 with 1 N HCl (aq.). The aqueous layer was extracted with DCM (3×30 mL). The combined organic layers were washed with H₂O (30 mL), dried over anhydrous Na₂SO₄. After filtration, the filtrate was concentrated under reduced pressure to afford the product (1.2 g, 84% yield) as a solid. LCMS (ESI) m/z [M+H] calcd for C₅₀H₆₆F₃N₇O₇: 934.51; found: 935.0

### Step 6: Synthesis of tert-butyl ((6³S,4S)-12-(2-((S)-1-methoxyethyl)-5-((R)-octahydro-2H-pyrido[1,2-a]pyrazin-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a stirred mixture of (S)-1-((S)-2-((tert-butoxycarbonyl)amino)-3-(3-(3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1-(2,2,2-trifluoroethyl)-1H-indol-5-yl)phenyl)propanoyl)hexahydropyridazine-3-carboxylic acid (1.2 g, 1.285 mmol) and DIPEA (7.83 mL, 44.975 mmol) in DCM (100 mL) were added HOBt (0.87 g, 6.425 mmol) and EDCI⋅HCl (5.58 g, 35.980 mmol) in portions at 0° C. The resulting mixture was stirred overnight at 0° C. The mixture was diluted with DCM (30 mL). The combined organic layers were washed with H₂O (3×30 mL), dried over anhydrous Na₂SO₄. After filtration, the filtrate was concentrated under reduced pressure. The residue was purified by silica gel column chromatography (5% MeOH/DCM) to afford the product (850 mg, 65% yield) as a solid. LCMS (ESI) m/z [M+H] calcd for C₅₀H₆₄F₃N₇O₆: 916.49; found: 917.0

### Step 7: Synthesis of (6³S,4S)-4-amino-12-(2-((S)-1-methoxyethyl)-5-((R)-octahydro-2H-pyrido[1,2-a]pyrazin-2-yl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a stirred mixture tert-butyl ((63S,4S)-12-(2-((S)-1-methoxyethyl)-5-((R)-octahydro-2H-pyrido[1,2-a]pyrazin-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1000 mg, 1.092 mmol) in DCM (4 mL) was added TFA (4 mL) at 0° C. The resulting mixture was stirred for 1 h at 0° C. under a nitrogen atmosphere. The resulting mixture was concentrated under reduced pressure. The residue was basified to pH 8 with sat. aq. NaHCO₃. The aqueous layer was extracted with DCM (3×30 mL). The combined organic layers dried over anhydrous Na₂SO₄. After filtration, the filtrate was concentrated under reduced pressure to afford the product (800 mg, 80% yield) as a solid. LCMS (ESI) m/z [M+H] calcd for C₄₅H₅₆F₃N₇O₄: 816.44 found: 816.6

### Intermediate F16: Synthesis of (6³S,4S)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

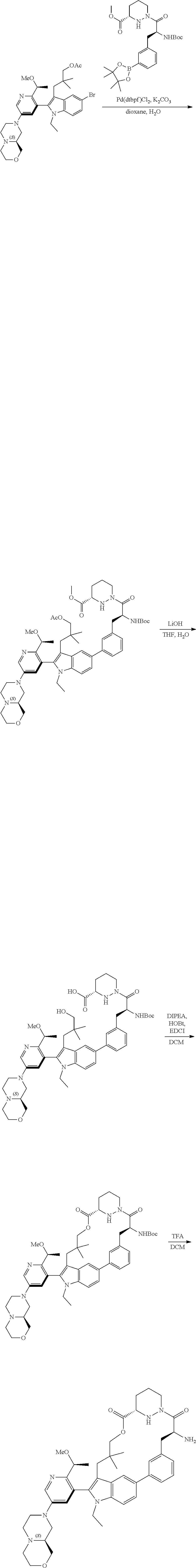

### Step 1: Synthesis of methyl (S)-1-((S)-3-(3-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)phenyl)-2-((tert-butoxycarbonyl)amino)propanoyl)hexahydropyridazine-3-carboxylate

Into a 500 mL 3-necked round-bottom flask were added 3-(5-bromo-1-ethyl-2-(5-((S)-hexahydropyrazino[2,1-c][1,4]oxazin-8(1H)-yl)-2-((S)-1-methoxyethyl)pyridin-3-yl)-1H-indol-3-yl)-2,2-dimethylpropyl acetate (14.2 g, 22.625 mmol), methyl (S)-1-((S)-2-((tert-butoxycarbonyl)amino)-3-(3-(4,4,5,5-tetramethyl-1,3,2-dioxaborolan-2-yl)phenyl)propanoyl)hexahydropyridazine-3-carboxylate (17.56 g, 33.938 mmol), H₂O (30 mL,) in dioxane (150 mL), Pd(dtbpf)Cl₂ (1.47 g, 2.263 mmol) at room temperature under an argon atmosphere. The resulting mixture was stirred for 3 h at 65° C. and then cooled to room temperature. The mixture was filtered, the filter cake was washed with EtOAc (2×200 mL). The filtrate was concentrated under reduced pressure and was then extracted with EtOAc (2×200 mL). The combined organic layers were washed with brine (2×250 mL) and dried over anhydrous Na₂SO₄. After filtration, the filtrate was concentrated under reduced pressure. The residue was purified by silica gel column chromatography (3→4% MeOH/DCM) to afford the product (17.2 g, 81% yield) as a solid. LCMS (ESI) m/z [M+H] calcd for C₅₂H₇₁N₇O₉: 938.54; found: 938.8

### Step 2: Synthesis of (S)-1-((S)-2-((tert-butoxycarbonyl)amino)-3-(3-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)phenyl)propanoyl)hexahydropyridazine-3-carboxylic acid

Into a 250 mL 3-necked round-bottom flask were added methyl (S)-1-((S)-3-(3-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)phenyl)-2-((tert-butoxycarbonyl)amino)propanoyl)hexahydropyridazine-3-carboxylate (17.2 g, 18.33 mmol) and THE (175 mL) at room temperature. To a stirred mixture of LiOH (1.88 g, 78.343 mmol) in H₂O (78.34 mL, 4348.526 mmol) in portions at 0° C. The resulting mixture was stirred for 16 h at room temperature. The mixture was acidified to pH 5 with 1 N HCl. The resulting mixture was extracted with EtOAc (2×200 mL). The combined organic layers were washed with brine (2×200 mL), dried over anhydrous Na₂SO₄. After filtration, the filtrate was concentrated under reduced pressure. The crude mixture (17 g, crude) as a solid was used in the next step directly without further purification. LCMS (ESI) m/z [M+H] calcd for C₄₉H₆₇N₇O₈: 882.51; found: 882.8

### Step 3: Synthesis of tert-butyl ((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

Into a 5 L 3-necked round-bottom flask were added (S)-1-((S)-2-((tert-butoxycarbonyl)amino)-3-(3-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)phenyl)propanoyl)hexahydropyridazine-3-carboxylic acid (16.8 g, 19.045 mmol) and DCM (2.52 L) at room temperature. To the above mixture was added DIPEA (98.46 g, 761.800 mmol) and HOBt (25.73 g, 190.450 mmol) in portions 0° C. The resulting mixture was stirred for additional 30 min at 0° C. Followed by the addition of EDCI (109.53 g, 571.350 mmol) in portions at 0° C. The mixture was stirred for 16 h at room temperature then concentrated under reduced pressure. The reaction was quenched with cold H₂O (500 mL) at 0° C. and extracted with EtOAc (3×500 mL). The combined organic layers were washed with brine (3×500 mL), dried over anhydrous Na₂SO₄. After filtration, the filtrate was concentrated under reduced pressure and purified by silica gel column chromatography (3→4% MeOH/DCM) to afford the product (13.4 g, 81% yield) as a solid. LCMS (ESI) m/z [M+H] calcd for C₄₉H₆₅N₇O₇: 864.50; found: 864.8

### Step 4: Synthesis of (6³S,4S)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

Into a 100 mL round-bottom flask were added tert-butyl ((6³S,4S)-11-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamatebutyl (300 mg, 0.347 mmol) and DCM (3 mL), TFA (1.5 mL) was added to the above solution at 0° C. After 1 h, the mixture was basified to pH 9 with sat. aq. NaHCO₃. The resulting mixture was extracted with DCM (3×50 mL). The combined organic layers were washed with brine (3×50 mL), dried over anhydrous Na₂SO₄. After filtration, the filtrate was concentrated under reduced pressure to afford the product (242 mg, crude) as a solid. The crude product was used in the next step directly without further purification. LCMS (ESI) m/z [M+H] calcd for C₄₄H₅₇N₇O₅: 764.45; found: 764.4

### Intermediate F17: Synthesis of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

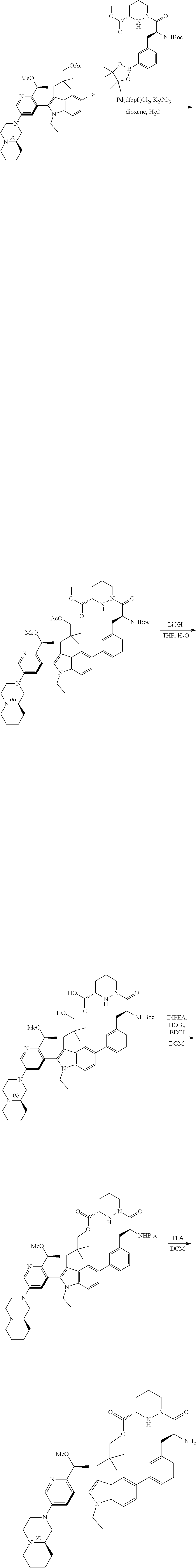

### Step 1: Synthesis of methyl (S)-1-((S)-3-(3-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)phenyl)-2-((tert-butoxycarbonyl)amino)propanoyl)hexahydropyridazine-3-carboxylate

Into a 40 mL vial were added 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (2 g, 3.196 mmol), methyl (3S)-1-[(2S)-2-[(tert-butoxycarbonyl)amino]-3-[3-(4,4,5,5-tetramethyl-1,3,2-dioxaborolan-2-yl)phenyl]propanoyl]-1,2-diazinane-3-carboxylate (1.98 g, 3.836 mmol) and H₂O (5 mL) in dioxane (20 mL) at room temperature. To the above mixture was added K₂CO₃ (883 mg, 6.392 mmol) and Pd(dtbpf)Cl₂ (208 mg, 0.32 mmol) in portions. The resulting mixture was stirred for additional 4 h at 65° C., then filtered, and the filter cake was washed with EtOAc (2×200 mL). The filtrate was concentrated under reduced pressure. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by prep-TLC (50% MeOH/DCM) to afford the product (2.11 g, 70% yield) as a solid. LCMS (ESI) m/z [M+H] calcd for C₅₃H₇₃N₇O₈: 936.56; found: 936.7

### Step 2: Synthesis of (S)-1-((S)-2-((tert-butoxycarbonyl)amino)-3-(3-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)phenyl)propanoyl)hexahydropyridazine-3-carboxylic acid

Into a 100 mL 3-necked round-bottom flask were added methyl (S)-1-((S)-3-(3-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)phenyl)-2-((tert-butoxycarbonyl)amino)propanoyl)hexahydropyridazine-3-carboxylate (2.1 g, 2.245 mmol) and THE (21 mL) at room temperature. To the above mixture was added LiOH⋅H₂O (283 mg, 6.735 mmol) in H₂O (7 mL) in portions at 0° C. The resulting mixture was stirred overnight at room temperature. The mixture was acidified to pH 5 with 1 N HCl. The resulting mixture was extracted with EtOAc (3×150 mL). The combined organic layers were washed with brine (2×150 mL), dried over anhydrous Na₂SO₄. After filtration, the filtrate was concentrated under reduced pressure to afford the product (1.8 g, crude). LCMS (ESI) m/z [M+H] calcd for C₅₀H₆₉N₇O₇: 880.53; found: 880.8

### Step 3: Synthesis of tert-butyl ((63S,4S)-1¹-ethyl-1²-(2-((S)-1-methoxyethyl)-5-((R)-octahydro-2H-pyrido[1,2-a]pyrazin-2-yl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

Into a 2 L 3-necked round-bottom flask were added (S)-1-((S)-2-((tert-butoxycarbonyl)amino)-3-(3-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)phenyl)propanoyl)hexahydropyridazine-3-carboxylic acid (1.8 g, 2.09 mmol) and DCM (360 mL) at room temperature followed by DIPEA (24.28 mL, 139.394 mmol) and HOBt (4.71 g, 34.857 mmol) in portions at 0° C. The resulting mixture was stirred for additional 30 min at 0° C. To the above mixture was added EDCI (8.67 g, 37.63 mmol) in portions over 5 min at 0° C. The resulting mixture was stirred overnight at room temperature then concentrated under reduced pressure. The reaction was quenched with H₂O at 0° C. The resulting mixture was extracted with EtOAc (3×300 mL). The combined organic layers were washed with brine (2×250 mL), dried over anhydrous Na₂SO₄.

After filtration, the filtrate was concentrated under reduced pressure. The residue was purified by silica gel column chromatography (10% MeOH/DCM) to the product (623 mg, 34% yield) as a solid. LCMS (ESI) m/z [M+H] calcd for C₅₁H₆₈N₆O₆: 861.53; found: 862.8

### Step 4: Synthesis of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

Into a 100 mL round-bottom flask were added tert-butyl ((6³S,4S)-11-ethyl-1²-(2-((S)-1-methoxyethyl)-5-((R)-octahydro-2H-pyrido[1,2-a]pyrazin-2-yl)phenyl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (350 mg, 0.406 mmol) and DCM (4 mL) at 0° C. Then TFA (1 mL) was added into above mixture. After 1 h, the mixture was basified to pH 9 with sat. aq. NaHCO₃. The resulting mixture was extracted with DCM (3×5 mL). The combined organic layers were washed with brine (3×30 mL), dried over anhydrous Na₂SO₄. After filtration, the filtrate was concentrated under reduced pressure to afford the product (300 mg, crude). LCMS (ESI) m/z [M+H] calcd for C₄₅H₅₉N₇O₄: 762.47; found: 762.3

### Intermediate F18: Synthesis of (6³S,4S)-4-amino-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-6¹,6²,6³,6⁴,6⁵,6⁶-hexahydro-1H-8-oxa-1(5,3)-indola-6(1,3)-pyridazina-2(1,3)-benzenacycloundecaphane-5,7-dione

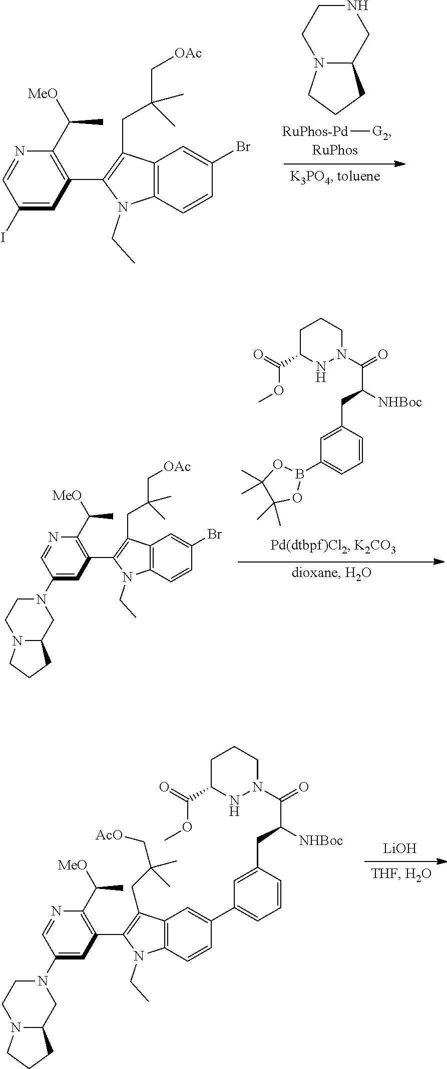

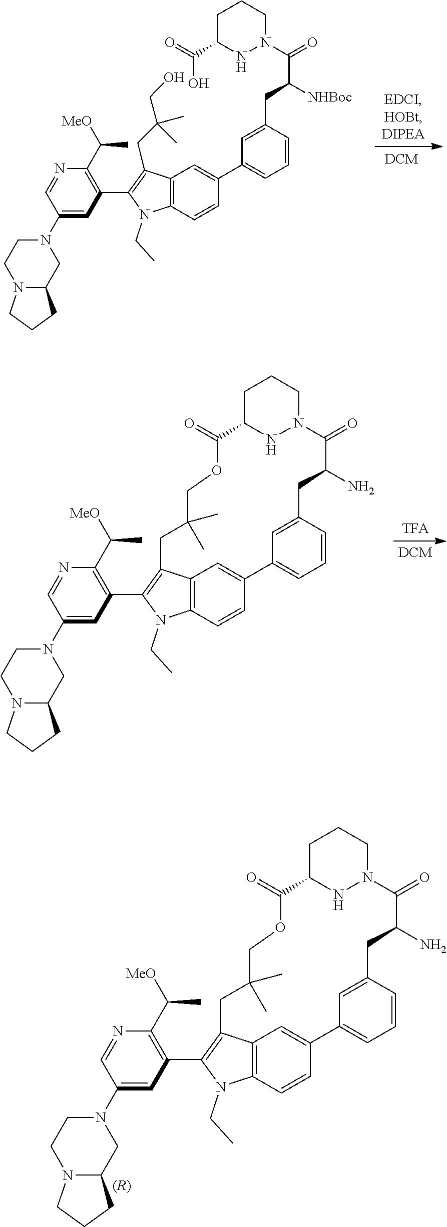

### Step 1: Synthesis of 3-(5-bromo-1-ethyl-2-(5-((R)-hexahydropyrrolo[1,2-a]pyrazin-2(1H)-yl)-2-((S)-1-methoxyethyl)pyridin-3-yl)-1H-indol-3-yl)-2,2-dimethylpropyl acetate

Into a 100 mL 3-necked round-bottom flask were added (S)-3-(5-bromo-1-ethyl-2-(5-iodo-2-(1-methoxyethyl)pyridin-3-yl)-1H-indol-3-yl)-2,2-dimethylpropyl acetate (10 g, 16.304 mmol), K₃PO₄ (8.65 g, 40.760 mmol), (R)-octahydropyrrolo[1,2-a]pyrazine (2.67 g, 21.195 mmol) in toluene (100 mL) at room temperature. To the above mixture was added RuPhos-Pd-G2 (2.53 g, 3.261 mmol), RuPhos (2.28 g, 4.891 mmol) in portions over 1 min. The resulting mixture was stirred for additional 3 h at 90° C. The reaction was quenched by the addition of H₂O (50 mL) at room temperature. The aqueous layer was extracted with EtOAc (3×600 mL). The resulting mixture was concentrated under reduced pressure. The residue was purified by silica gel column chromatography (12% MeOH/DCM) to afford the product (7 g, 70% yield) as a solid. LCMS (ESI) m/z [M+H] calcd for C₃₂H₄₃BrN₄O₃: 611.26; found: 611.3

### Step 2: Synthesis of methyl (S)-1-((S)-3-(3-(3-(3-acetoxy-2,2-dimethylpropyl)-1-ethyl-2-(5-((R)-hexahydropyrrolo[1,2-a]pyrazin-2(1H)-yl)-2-((S)-1-methoxyethyl)pyridin-3-yl)-1H-indol-5-yl)phenyl)-2-((tert-butoxycarbonyl)amino)propanoyl)hexahydropyridazine-3-carboxylate

To a solution of 3-(5-bromo-1-ethyl-2-(5-((R)-hexahydropyrrolo[1,2-a]pyrazin-2(1H)-yl)-2-((S)-1-methoxyethyl)pyridin-3-yl)-1H-indol-3-yl)-2,2-dimethylpropyl acetate (5 g, 8.175 mmol) and methyl (3S)-1-[(2S)-2-[(tert-butoxycarbonyl)amino]-3-[3-(4,4,5,5-tetramethyl-1,3,2-dioxaborolan-2-yl)phenyl]propanoyl]-1,2-diazinane-3-carboxylate (5.07 g, 9.81 mmol) in dioxane (125 mL) and H₂O (25 mL) were added K₂CO₃ (2824 mg, 20.438 mmol) and Pd(dppf)Cl₂ (1196 mg, 1.635 mmol). After stirring for 2 h at 70° C. under a nitrogen atmosphere. The precipitated solids were collected by filtration and washed with EtOAc (3×100 mL). The resulting mixture was concentrated under reduced pressure. The residue was purified by silica gel column chromatography (13% MeOH/DCM) to afford the product (4 g, 53% yield) as a solid.

LCMS (ESI) m/z [M+H] calcd for C₅₂H₇₁N₇O₈: 922.54; found: 922.6

### Step 3: Synthesis of (S)-1-((S)-2-((tert-butoxycarbonyl)amino)-3-(3-(1-ethyl-2-(5-((R)-hexahydropyrrolo[1,2-a]pyrazin-2(1H)-yl)-2-((S)-1-methoxyethyl)pyridin-3-yl)-3-(3-hydroxy-2,2-dimethylpropyl)-1H-indol-5-yl)phenyl)propanoyl)hexahydropyridazine-3-carboxylic acid

Into a 100 mL round-bottom flask were added methyl (S)-1-((S)-3-(3-(3-(3-acetoxy-2,2-dimethylpropyl)-1-ethyl-2-(5-((R)-hexahydropyrrolo[1,2-a]pyrazin-2(1H)-yl)-2-((S)-1-methoxyethyl)pyridin-3-yl)-1H-indol-5-yl)phenyl)-2-((tert-butoxycarbonyl)amino)propanoyl)hexahydropyridazine-3-carboxylate (3 g, 3.2501 mmol) and THE (30 mL) and LiOH⋅H₂O (0.55 g, 13.012 mmol) at 0° C. The resulting mixture was stirred overnight at room temperature under an argon atmosphere. The mixture was acidified to pH 6 with HCl (aq.). The aqueous layer was extracted with EtOAc (3×100 mL). The resulting mixture was concentrated under reduced pressure to afford the product (2.96 g, 92% yield) as a solid. LCMS (ESI) m/z [M+H] calcd for C₄₉H₆₇N₇O₇: 866.52; found: 866.6

### Step 4: Synthesis of tert-butyl ((63S,4S)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

Into a 500 mL round-bottom flask were added (S)-1-((S)-2-((tert-butoxycarbonyl)amino)-3-(3-(1-ethyl-2-(5-((R)-hexahydropyrrolo[1,2-a]pyrazin-2(1H)-yl)-2-((S)-1-methoxyethyl)pyridin-3-yl)-3-(3-hydroxy-2,2-dimethylpropyl)-1H-indol-5-yl)phenyl)propanoyl)hexahydropyridazine-3-carboxylic acid (2.9 g, 3.348 mmol), DCM (200 mL), and DIPEA (8.65 g, 66.960 mmol) in at 0° C. To the above mixture was added HOBt (2.26 g, 16.740 mmol), EDCI (6.42 g, 33.480 mmol) in portions over 5 min at 0° C. The resulting mixture was stirred overnight at room temperature. The resulting mixture was washed with H₂O (3×200 mL). The aqueous layer was extracted with EtOAc (3×20 mL). The resulting mixture was concentrated under reduced pressure. The residue was purified by silica gel column chromatography (10% MeOH/DCM) to afford the product (1.2 g, 42% yield) as a solid. LCMS (ESI) m/z [M+H] calcd for C₄₉H₆₅N₇O₆: 848.51; found: 847.6

### Step 5: Synthesis of (6³S,4S)-4-amino-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

Into a 40 mL vial were added tert-butyl ((6³S,4S)-1′-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (400 mg, 0.472 mmol) DCM (8 mL), and TFA (4 mL) at 0° C. The resulting mixture was stirred for 3 h at 0° C. The resulting mixture was concentrated under reduced pressure to afford the product (300 mg, 85% yield) as a solid. LCMS (ESI) m/z [M+H] calcd for C₄₄H₅₇N₇O₄: 747.4; found: 748.4

### Intermediate F19: Synthesis of (2²S,6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1H-8-oxa-2(4,2)-morpholina-1(5,3)-indola-6(1,3)-pyridazinacycloundecaphane-5,7-dione

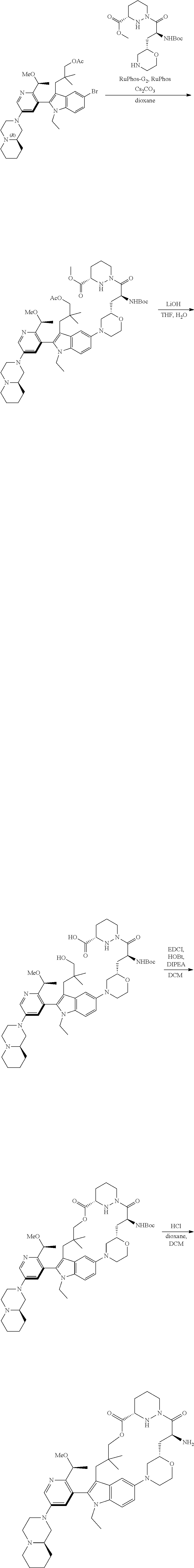

### Step 1: Synthesis of methyl (S)-1-((S)-3-((S)-1-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)piperidin-3-yl)-2-((tert-butoxycarbonyl)amino)propanoyl)hexahydropyridazine-3-carboxylate

To a stirred solution of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (13 g, 20.778 mmol) and methyl (S)-1-((S)-2-((tert-butoxycarbonyl)amino)-3-((S)-morpholin-2-yl)propanoyl)hexahydropyridazine-3-carboxylate (10.82 g, 27.011 mmol) in dioxane (130 mL) were added chloro(2-dicyclohexylphosphino-2′,6′-diisopropoxy-1,1′-biphenyl)[2-(2′-amino-1,1′-biphenyl)]palladium(II) (1.61 g, 2.078 mmol), RuPhos (1.94 g, 4.156 mmol) and Cs₂CO₃ (13.54 g, 41.556 mmol) at room temperature under a nitrogen atmosphere. The resulting mixture was stirred for 3 h at 80° C. The resulting mixture was diluted with H₂O (300 mL) and extracted with EtOAc (3×200 mL). The combined organic layers were washed with brine (2 20×100 mL), dried over anhydrous Na₂SO₄. After filtration, the filtrate was concentrated under reduced pressure. The residue was purified by silica gel column chromatography (8% MeOH/DCM) to afford the product (18.8 g, 95% yield) as a solid. LCMS (ESI) m/z [M+H] calcd for C₅₂H₇₈N₈O₈: 945.58; found: 945.5

### Step 2: Synthesis of (S)-1-((S)-2-((tert-butoxycarbonyl)amino)-3-((S)-4-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)morpholin-2-yl)propanoyl)hexahydropyridazine-3-carboxylic acid

To a stirred solution of methyl (S)-1-((S)-3-((S)-1-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)piperidin-3-yl)-2-((tert-butoxycarbonyl)amino)propanoyl)hexahydropyridazine-3-carboxylate (18.8 g, 19.890 mmol) in THE (85 mL) and H₂O (85 mL) was added LiOH⋅H₂O (4.17 g, 99.450 mmol) at 0° C. The resulting mixture was stirred at room temperature then diluted with H₂O (300 mL). The resulting mixture was washed with MTBE (3×100 mL) and extracted with DCM (3×200 mL). The combined organic layers were washed with H₂O (200 mL), dried over anhydrous Na₂SO₄. After filtration, the filtrate was concentrated under reduced pressure to afford the product (11.2 g, crude) as a solid. The crude product was used in the next step directly without further purification. LCMS (ESI) m/z [M+H] calcd for C₄₈H₇₂N₈O₈: 889.56; found: 889.5

### Step 3: Synthesis of tert-butyl ((2²S,6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate

To a stirred solution of (S)-1-((S)-2-((tert-butoxycarbonyl)amino)-3-((S)-4-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)morpholin-2-yl)propanoyl)hexahydropyridazine-3-carboxylic acid (5.57 g, 6.264 mmol) and DIPEA (64.77 g, 501.120 mmol) in DMF (557 mL) were added HOBt (33.86 g, 250.560 mmol) and EDCI (72.05 g, 375.840 mmol) at 0° C. under a nitrogen atmosphere. The resulting mixture was stirred overnight at room temperature. The resulting mixture was diluted with H₂O (1 L). The resulting mixture was extracted with EtOAc (3×1 L). The combined organic layers were washed with brine (5×1 L), dried over anhydrous Na₂SO₄. After filtration, the filtrate was concentrated under reduced pressure. The residue was purified by silica gel column chromatography (10% MeOH/DCM) to afford the product (4 g, 73% yield) as a solid. LCMS (ESI) m/z [M+H] calcd for C₄₈H₇₀N₈O₇: 871.54; found: 871.6

### Step 4: Synthesis of (2²S,6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

To a stirred mixture of tert-butyl ((2²S,6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate (750 mg, 0.861 mmol) in DCM (5 mL) was added HCl (4 M in dioxane) (5 mL) dropwise at 0° C. The resulting mixture was stirred for 2 h at room temperature then concentrated under reduced pressure. This resulted in the product (830 mg, crude) as a solid. LCMS (ESI) m/z [M+H] calcd for C₄₃H₆₂N₈O₅: 771.49; found: 771.7

### Intermediate F20: Synthesis of (6³S,4S)-4-amino-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

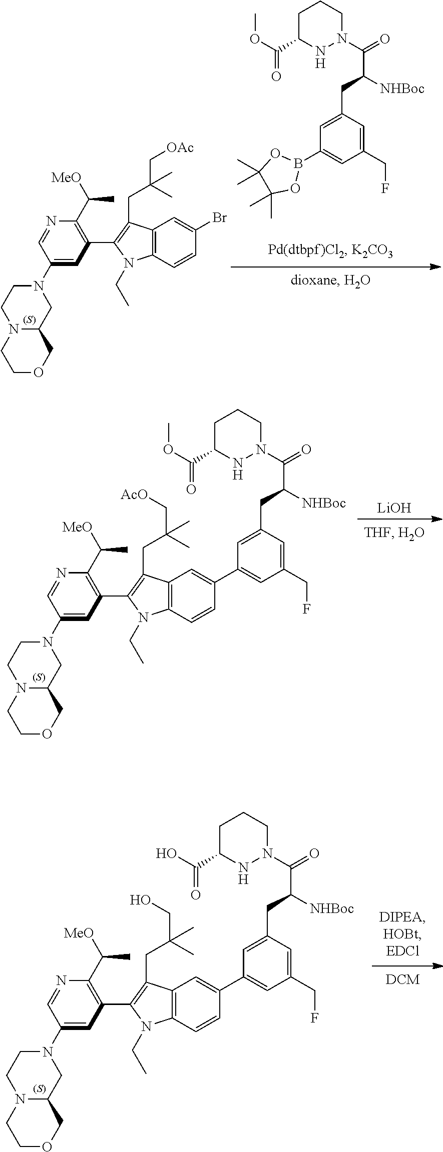

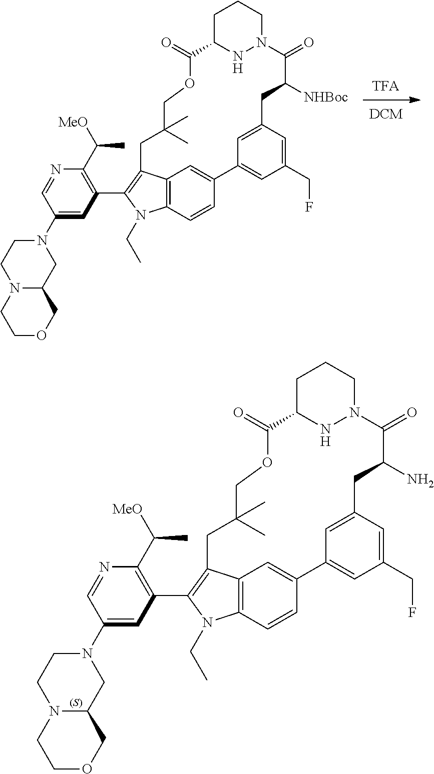

### Step 1: Synthesis of methyl (S)-1-((S)-3-(3-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)-5-(fluoromethyl)phenyl)-2-((tert-butoxycarbonyl)amino)propanoyl)hexahydropyridazine-3-carboxylate

To a solution of 3-(5-bromo-1-ethyl-2-(5-((S)-hexahydropyrazino[2,1-c][1,4]oxazin-8(1H)-yl)-2-((S)-1-methoxyethyl)pyridin-3-yl)-1H-indol-3-yl)-2,2-dimethylpropyl acetate (700 mg, 1.115 mmol), K₃PO₄ (592 mg, 2.788 mmol) and methyl (3S)-1-[(2S)-2-[(tert-butoxycarbonyl)amino]-3-[3-(fluoromethyl)-5-(4,4,5,5-tetramethyl-1,3,2-dioxaborolan-2-yl)phenyl]propanoyl]-1,2-diazinane-3-carboxylate (1.225 g, 2.230 mmol) in toluene (6 mL), dioxane (4 mL) and H₂O (2 mL) was added Pd(dtbpf)Cl₂ (73 mg, 0.112 mmol) at room temperature under a nitrogen atmosphere. The resulting mixture was stirred for 2 h at 70° C. The mixture was concentrated under reduced pressure, diluted with H₂O (200 mL), and extracted with (DCM:MeOH 10%) (4×100 mL). The combined organic layers were washed with brine (3×200 mL), dried over anhydrous Na₂SO₄. After filtration, the filtrate was concentrated under reduced pressure. The residue was purified by silica gel column chromatography (10% MeOH/DCM) to afford the product (1.062 g, 98% yield) as a solid. LCMS (ESI) m/z [M+H] calcd for C₅₃H₇₂FN₇O₉: 970.55; found: 970.4

### Step 2: Synthesis of (S)-1-((S)-2-((tert-butoxycarbonyl)amino)-3-(3-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)-5-(fluoromethyl)phenyl)propanoyl)hexahydropyridazine-3-carboxylic acid

To a solution of methyl (S)-1-((S)-3-(3-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)-5-(fluoromethyl)phenyl)-2-((tert-butoxycarbonyl)amino)propanoyl)hexahydropyridazine-3-carboxylate (1 g, 1.031 mmol) in THE (5 mL) and H₂O (5 mL) were added LiOH (0.12 g, 5.155 mmol) at 0° C. The resulting mixture was stirred overnight. The mixture was adjusted to pH 7 with 0.5N HCl (aq.). The resulting mixture was extracted with 10% MeOH/DCM (4×100 mL) and the combined organic layers were washed with brine (5×20 mL) and dried over anhydrous Na₂SO₄. After filtration, the filtrate was concentrated under reduced pressure to afford the product (810 mg, 86% yield) as a solid. LCMS (ESI) m/z [M+H] calcd C₅₀H₆₈FN₇O₈: 914.52; found: 914.4

### Step 3: Synthesis of tert-butyl ((6³S,4S)-1¹-ethyl-16-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of (S)-1-((S)-2-((tert-butoxycarbonyl)amino)-3-(3-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)-5-(fluoromethyl)phenyl)propanoyl)hexahydropyridazine-3-carboxylic acid (620 mg, 0.678 mmol) and DIPEA (3068 mg, 23.730 mmol) in DCM (60 mL) were added EDCI (3640.55 mg, 18.984 mmol) and HOBt (458 mg, 3.390 mmol) at 0° C. The resulting mixture was stirred overnight. The mixture was washed with brine (5×20 mL) and dried over anhydrous Na₂SO₄. After filtration, the filtrate was concentrated under reduced pressure. The residue was purified by reverse phase chromatography (0→100% MeCN/H₂O, 0.1% NH₄HCO₃) to afford the product (420 mg, 69% yield) as a solid. LCMS (ESI) m/z [M+H] calcd for C₅₀H₆₆FN₇O₇: 896.51; found: 896.7

### Step 4: Synthesis of (6³S,4S)-4-amino-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a solution of tert-butyl ((63S,4S)-1¹-ethyl-16-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (420 mg, 0.468 mmol) in DCM (5 mL) was added TFA (2.5 mL) at 0° C. The resulting mixture was stirred for 1 h at 0° C. The mixture was concentrated under reduced pressure and acidified to pH 7 with sat. aq. NaHCO₃. The resulting mixture was extracted with 10% MeOH/DCM (4×100 mL). The combined organic layers were washed with brine (3×50 mL) and dried over anhydrous Na₂SO₄. After filtration, the filtrate was concentrated under reduced pressure to afford the product (400 mg, 95% yield) as a solid. LCMS (ESI) m/z [M+H] calcd for C₄₅H₅₈FN₇O₅: 796.46; found: 796.2

### Intermediate F21: Synthesis of (6³S,4S)-4-amino-25-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

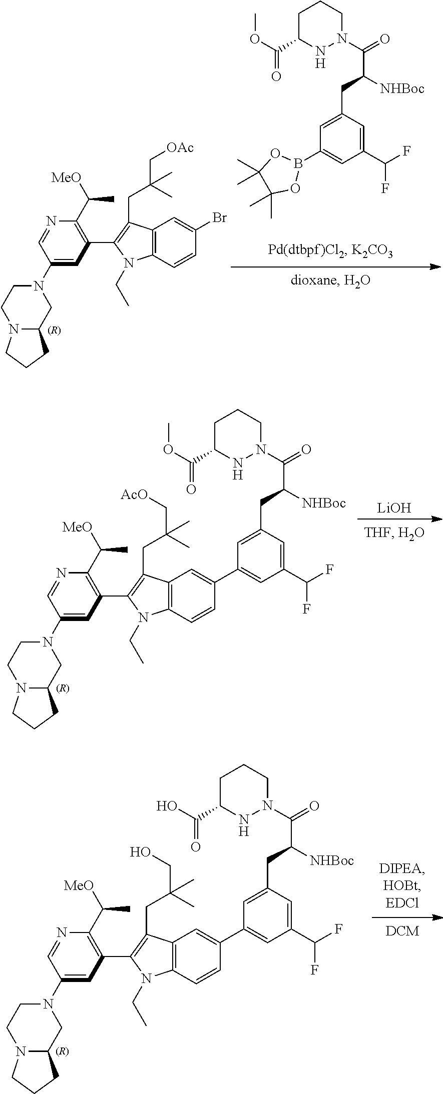

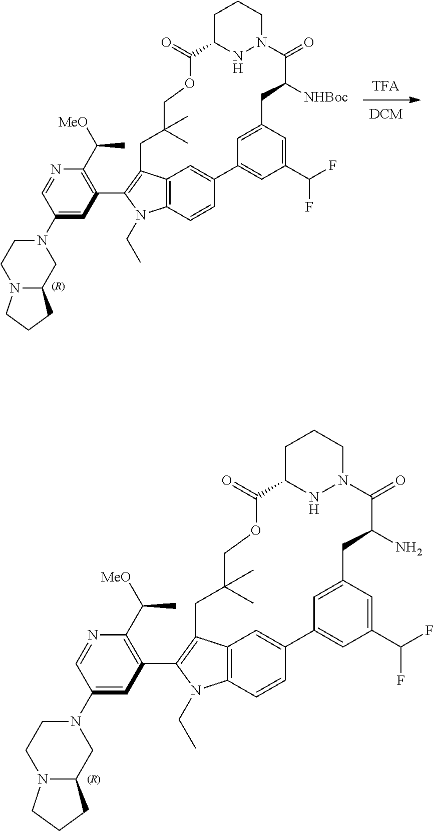

### Step 1: Synthesis of methyl (S)-1-((S)-3-(3-(3-(3-acetoxy-2,2-dimethylpropyl)-1-ethyl-2-(5-((R)-hexahydropyrrolo[1,2-a]pyrazin-2(1H)-yl)-2-((S)-1-methoxyethyl)pyridin-3-yl)-1H-indol-5-yl)-5-(difluoromethyl)phenyl)-2-((tert-butoxycarbonyl)amino)propanoyl)hexahydropyridazine-3-carboxylate

To a stirred solution of 3-(5-bromo-1-ethyl-2-(5-((R)-hexahydropyrrolo[1,2-a]pyrazin-2(1H)-yl)-2-((S)-1-methoxyethyl) pyridin-3-yl)-1H-indol-3-yl)-2,2-dimethylpropyl acetate (2 g, 3.270 mmol), methyl (S)-1-((S)-2-((tert-butoxycarbonyl)amino)-3-(3-(difluoromethyl)-5-(4,4,5,5-tetramethyl-1,3,2-dioxaborolan-2-yl)phenyl)propanoyl) hexahydropyridazine-3-carboxylate (2.78 g, 4.905 mmol), K₃PO₄ (2.08 g, 9.810 mmol) and Pd(DtBPF)Cl₂ (0.43 g, 0.654 mmol) was added dioxane (50 mL) and H₂O (10 mL) under an argon atmosphere. The resulting mixture was stirred for 2 h at 70° C. The mixture cooled to room temperature, quenched by the addition of cold H₂O (30 mL) and extracted with EtOAc (100 mL). The combined organic layers were washed with brine (3×50 mL) and dried over anhydrous Na₂SO₄. After filtration, the filtrate was concentrated under reduced pressure. The residue was purified by silica gel column chromatography (20% MeOH/DCM) to afford the product (1.70 g, 53% yield) as an oil. LCMS (ESI) [M+H] calcd for C₅₃H₇₁F₂N₇O₈: 972.54; found: 973.1

### Step 2: Synthesis of (S)-1-((S)-2-((tert-butoxycarbonyl)amino)-3-(3-(difluoromethyl)-5-(1-ethyl-2-(5-((R)-hexahydropyrrolo[1,2-a]pyrazin-2(1H)-yl)-2-((S)-1-methoxyethyl)pyridin-3-yl)-3-(3-hydroxy-2,2-dimethylpropyl)-1H-indol-5-yl)phenyl)propanoyl)hexahydropyridazine-3-carboxylic acid

Into a 100 mL round-bottom flask was added methyl (S)-1-((S)-3-(3-(3-(3-acetoxy-2,2-dimethylpropyl)-1-ethyl-2-(5-((R)-hexahydropyrrolo[1,2-a]pyrazin-2(1H)-yl)-2-((S)-1-methoxyethyl)pyridin-3-yl)-1H-indol-5-yl)-5-(difluoromethyl)phenyl)-2-((tert-butoxycarbonyl)amino)propanoyl)hexahydropyridazine-3-carboxylate (1.30 g, 1.316 mmol), LiOH⋅H₂O (0.33 g, 7.896 mmol), H₂O (15 mL), and THE (15 mL) at 0° C. The resulting mixture was stirred for 2 h at room temperature. The reaction mixture was filtered, the filtrate was concentrated under reduced pressure. The mixture was adjusted to pH 7 to 8 with 1M HCl at 0° C. The mixture was extracted with EtOAc (3×50 mL) and the combined organic layers were washed with brine (3×10 mL) and dried over anhydrous Na₂SO₄. After filtration, the filtrate was concentrated under reduced pressure. The residue was purified by silica gel column chromatography (20% EtOAc/pet. ether) to afford the product (0.96 g, 78% yield) as a solid. LCMS (ESI) m/z [M+H] calcd for C₅₀H₆₇F₂N₇O₇: 916.51; found: 916.5

### Step 3: Synthesis of tert-butyl ((6³S,4S)-25-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of (S)-1-((S)-2-((tert-butoxycarbonyl)amino)-3-(3-(difluoromethyl)-5-(1-ethyl-2-(5-((R)-hexahydropyrrolo[1,2-a]pyrazin-2(1H)-yl)-2-((S)-1-methoxyethyl)pyridin-3-yl)-3-(3-hydroxy-2,2-dimethylpropyl)-1H-indol-5-yl)phenyl)propanoyl)hexahydropyridazine-3-carboxylic acid (1.2 g, 1.310 mmol) and DIPEA (5.93 g, 45.850 mmol) in DCM (130 mL) was added HOBt (0.88 g, 6.550 mmol) and EDCI (7.03 g, 36.680 mmol) at 0° C. The resulting mixture was stirred for 1 h at room temperature under a nitrogen atmosphere. The mixture was extracted with DCM (3×20 mL) and the combined organic layers were washed with brine (3×20 mL) and dried over anhydrous Na₂SO₄. After filtration, the filtrate was concentrated under reduced pressure. The residue was purified by silica gel column chromatography (16% MeOH/DCM) to afford the product (0.915 g, 78% yield) as a solid. LCMS (ESI) m/z [M+H] calcd for C₅₀H₆₅F₂N₇O₆: 898.50; found: 898.1

### Step 4: Synthesis of (6³S,4S)-4-amino-25-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a solution of tert-butyl ((6³S,4S)-25-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (900 mg, 1.002 mmol) in DCM (10 mL) was added TFA (5 mL) at 0° C. The final reaction mixture was stirred for 1 h at room temperature under a nitrogen atmosphere. The mixture was then quenched by the addition of sat. aq. NaHCO₃ (30 mL) at 0° C. and extracted with EtOAc (100 mL). The combined organic layers were washed with brine (3×30 mL) and dried over anhydrous Na₂SO₄. After filtration, the filtrate was concentrated under reduced pressure to afford the product (586 mg, crude) as a solid, which was used in the next step directly without further purification. LCMS (ESI) m/z [M+H] calcd for C₄₅H₅₇F₂N₇O₄: 798.45; found: 798.2

### Intermediate F22: Synthesis of (6³S,4S)-4-amino-2⁵-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6-hexahydro-1¹H-8-oxa-1(5,3)-indola-6(1,3)-pyridazina-2(1,3)-benzenacycloundecaphane-5,7-dione

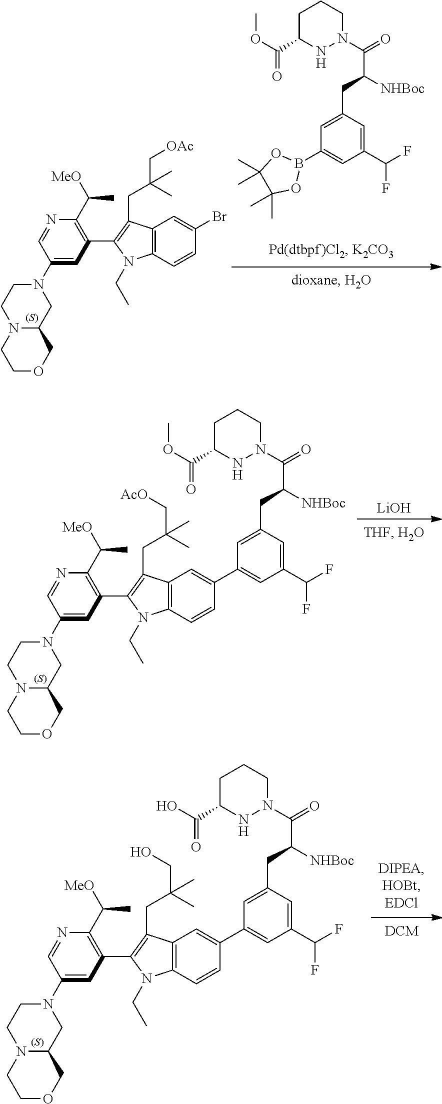

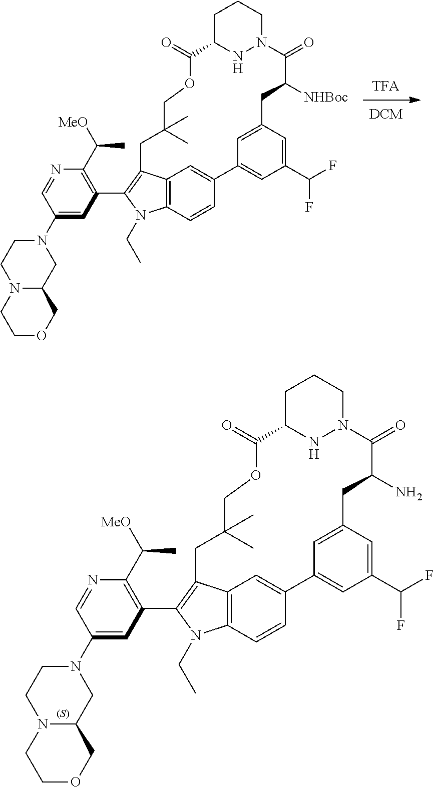

### Step 1: Synthesis of methyl (S)-1-((S)-3-(3-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)-5-(difluoromethyl)phenyl)-2-((tert-butoxycarbonyl)amino)propanoyl)hexahydropyridazine-3-carboxylate

To a stirred solution of 3-(5-bromo-1-ethyl-2-(5-((S)-hexahydropyrazino[2,1-c][1,4]oxazin-8(1H)-yl)-2-((S)-1-methoxyethyl)pyridin-3-yl)-1H-indol-3-yl)-2,2-dimethylpropyl acetate (1.00 g, 1.593 mmol), methyl (S)-1-((S)-2-((tert-butoxycarbonyl)amino)-3-(3-(difluoromethyl)-5-(4,4,5,5-tetramethyl-1,3,2-dioxaborolan-2-yl)phenyl) propanoyl)hexahydropyridazine-3-carboxylate (1.36 g, 2.389 mmol), K₃PO₄ (1.01 g, 4.779 mmol) and Pd(dtbpf)Cl₂ (207.69 mg, 0.319 mmol) was added dioxane (10 mL) and H₂O (2 mL) dropwise at room temperature under an argon atmosphere. The resulting mixture was stirred for 3 h at 70° C. The mixture was then cooled to room temperature, quenched by the addition of cold H₂O (30 mL), and extracted with EtOAc (100 mL). The combined organic layers were washed with brine (2×50 mL) and dried over anhydrous Na₂SO₄. After filtration, the filtrate was concentrated under reduced pressure. The residue was purified by silica gel column chromatography (16% MeOH/DCM) to afford the product (1.30 g, 83% yield) as a solid. LCMS (ESI) m/z [M+H] calcd for C₅₃H₇₁F₂N₇O: 988.54; found: 988.6

### Step 2: Synthesis of (S)-1-((S)-2-((tert-butoxycarbonyl)amino)-3-(3-(difluoromethyl)-5-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)phenyl)propanoyl)hexahydropyridazine-3-carboxylic acid

Into a 100 mL round-bottom flask was added a solution of methyl (S)-1-((S)-3-(3-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)-5-(difluoromethyl)phenyl)-2-((tert-butoxycarbonyl)amino)propanoyl)hexahydropyridazine-3-carboxylate (1.30 g, 1.316 mmol) in THE (10 mL) followed by a solution of LiOH⋅H₂O (0.33 g, 7.896 mmol) in H₂O (10 mL) at 0° C. The resulting mixture was stirred overnight at room temperature. The mixture was acidified to pH 7 to 8 with 0.5 M HCl at 0° C. The resulting mixture was extracted with 15% MeOH/DCM (3×50 mL) and the combined organic layers were washed with brine (3×10 mL) and dried over anhydrous Na₂SO₄. After filtration, the filtrate was concentrated under reduced pressure. The residue was purified by silica gel column chromatography (15% MeOH/DCM) to afford the product (1.20 g, crude) as a solid. LCMS (ESI) m/z [M+H] calcd for C₅₀H₆₇F₂N₇O₈: 932.51; found: 932.6

### Step 3: Synthesis of tert-butyl ((6³S,4S)-2⁵-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of (S)-1-((S)-2-((tert-butoxycarbonyl)amino)-3-(3-(difluoromethyl)-5-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)phenyl)propanoyl)hexahydropyridazine-3-carboxylic acid (1.40 g, 1.502 mmol) and DIPEA (6.79 g, 52.570 mmol) in DCM (140 mL) was added HOBt (1.01 g, 7.510 mmol) and EDCI (8.06 g, 42.056 mmol) at 0° C. The resulting mixture was stirred overnight at room temperature under a nitrogen atmosphere. The resulting mixture was extracted with DCM and the combined organic layers were washed with brine (3×20 mL) and dried over anhydrous Na₂SO₄. After filtration, the filtrate was concentrated under reduced pressure. The residue was purified by silica gel column chromatography (10% MeOH/DCM) to afford the product (579 mg, 42% yield) as a solid. LCMS (ESI) m/z [M+H] calcd for C₅₀H₆₅F₂N₇O₇: 914.50; found: 914.5

### Step 4: Synthesis of (6³S,4S)-4-amino-2⁵-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a solution of tert-butyl ((6³S,4S)-2⁵-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)carbamate (579.0 mg, 0.633 mmol) in DCM (6 mL) was added TFA (3 mL) at 0° C. The reaction mixture was stirred for 1 h at 0° C. The mixture was then concentrated under reduced pressure and adjusted to pH 8 by the addition of sat. aq. NaHCO₃ at 0° C. and extracted with 100 mL of (10% MeOH/DCM). The combined organic layers were washed with brine (3×50 mL) and dried over anhydrous Na₂SO₄. After filtration, the filtrate was concentrated under reduced pressure to afford the crude product (500 mg, crude) as a solid, which was used in the next step directly without further purification. LCMS (ESI) m/z [M+H] calcd for C₄₅H₅₇F₂N₇O₅: 814.45; found: 814.8

### Intermediate F23: Synthesis of (6³S,4S)-4-amino-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6-hexahydro-1¹H-8-oxa-1(5,3)-indola-6(1,3)-pyridazina-2(1,3)-benzenacycloundecaphane-5,7-dione

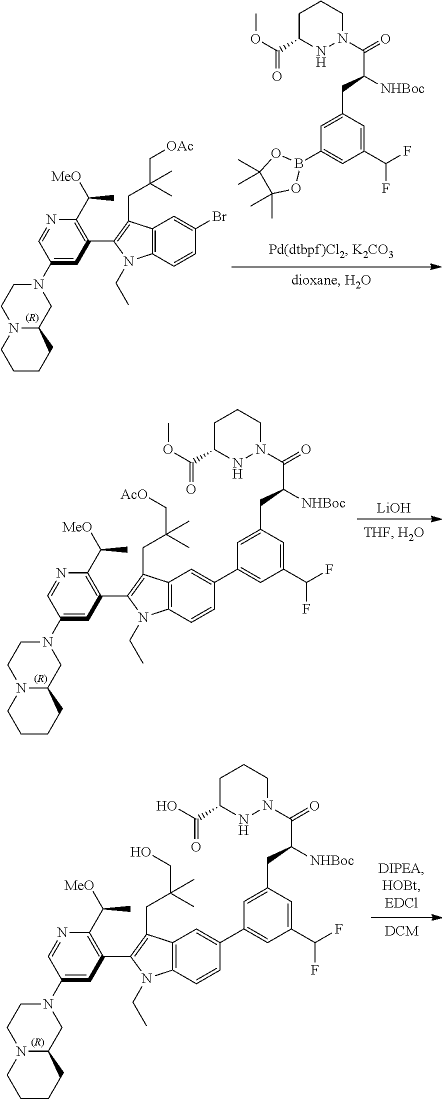

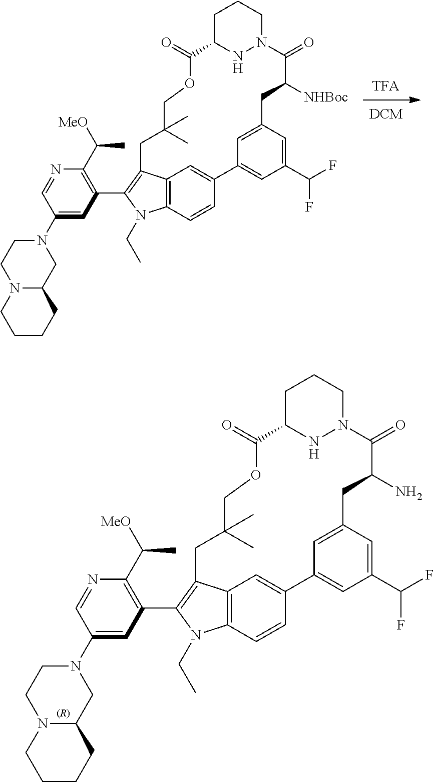

### Step 1: Synthesis of methyl (S)-1-((S)-3-(3-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)-5-(difluoromethyl)phenyl)-2-((tert-butoxycarbonyl)amino)propanoyl)hexahydropyridazine-3-carboxylate

To a stirred solution of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (1.50 g, 2.397 mmol), methyl (3S)-1-[(2S)-2-[(tert-butoxycarbonyl)amino]-3-[3-(difluoromethyl)-5-(4,4,5,5-tetramethyl-1,3,2-dioxaborolan-2-yl)phenyl]propanoyl]-1,2-diazinane-3-carboxylate (2.04 g, 3.595 mmol), K₃PO₄ (1.53 g, 7.191 mmol) and Pd(DtBPF)Cl₂ (0.31 g, 0.479 mmol) was added dioxane (20 mL) and H₂O (4 mL) under an argon atmosphere. The resulting mixture was stirred for 2 h at 70° C. The mixture was cooled to room temperature, quenched by the addition of cold H₂O (30 mL), and extracted with EtOAc (100 mL). The combined organic layers were washed with brine (3×50 mL) and dried over anhydrous Na₂SO₄. After filtration, the filtrate was concentrated under reduced pressure. The residue was purified by silica gel column chromatography (20% MeOH/DCM) to afford the product (1.20 g, 51% yield) as an oil. LCMS (ESI) m/z [M+H] calcd for C₅₄H₇₃F₂N₇O₈: 986.56; found: 986.5

### Step 2: Synthesis of (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)phenyl)propanoyl)hexahydropyridazine-3-carboxylic acid

Into a 100 mL round-bottom flask was added methyl (S)-1-((S)-3-(3-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)-5-(difluoromethyl)phenyl)-2-((tert-butoxycarbonyl)amino)propanoyl)hexahydropyridazine-3-carboxylate (1.20 g, 1.217 mmol), LiOH⋅H₂O (0.31 g, 7.302 mmol), H₂O (15 mL), and THE (15 mL) at 0° C. The resulting mixture was stirred for 2 h at room temperature. The reaction mixture was filtered, the filtrate was concentrated under reduced pressure. The mixture was adjusted to pH 7 to 8 with 1 M HCl at 0° C. and extracted with EtOAc (3×50 mL). The combined organic layers were washed with brine (3×10 mL) and dried over anhydrous Na₂SO₄. After filtration, the filtrate was concentrated under reduced pressure. The residue was purified by silica gel column chromatography (20% EtOAc/pet. ether) to afford the product (0.98 g, crude) as a solid.

LCMS (ESI) m/z [M+H] calcd for C₅₁H₆₉F₂N₇O₇: 930.53; found: 930.3

### Step 3: Synthesis of tert-butyl ((6³S,4S)-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of (S)-1-((S)-2-((tert-butoxycarbonyl)amino)-3-(3-(difluoromethyl)-5-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)phenyl)propanoyl)hexahydropyridazine-3-carboxylic acid (980 mg, 1.054 mmol) and DIPEA (4.09 g, 31.620 mmol) in DCM (200 mL) was added HOBt (711.82 mg, 5.270 mmol) and EDCI (5.05 g, 26.350 mmol) at 0° C. The resulting mixture was stirred for 1 h at room temperature under a nitrogen atmosphere. The mixture was extracted with DCM (3×20 mL). and the combined organic layers were washed with brine (3×50 mL) and dried over anhydrous Na₂SO₄. After filtration, the filtrate was concentrated under reduced pressure. The residue was purified by silica gel column chromatography (14% MeOH/DCM) to afford the product (869 mg, 90% yield) as a solid. LCMS (ESI) m/z [M+H] calcd for C₅₁H₆₇F₂N₇O₆: 912.52; found: 912.6

### Step 4: Synthesis of (6³S,4S)-4-amino-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a solution of tert-butyl ((6³S,4S)-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (869 mg, 0.953 mmol) in DCM (10 mL) was added TFA (5 mL) at 0° C. The final reaction mixture was stirred for 1 h at room temperature under a nitrogen atmosphere. The mixture was quenched by the addition of sat. aq. NaHCO₃ (30 mL) at 0° C. and extracted with EtOAC (100 mL). The combined organic layers were washed with brine (3×10 mL) and dried over anhydrous Na₂SO₄. After filtration, the filtrate was concentrated under reduced pressure. The crude product (706 mg, crude) as a solid was used in the next step directly without further purification. LCMS (ESI) m/z [M+H] calcd for C₄₆H₅₉F₂N₇O₄: 812.47; found: 812.4

### Intermediate F24: Synthesis of (6³S,4S)-4-amino-1¹-ethyl-12-(5-((S)-hexahydropyrazino[2,1-c][1,4]oxazin-8(1H)-yl)-2-((S)-1-methoxyethyl)pyridin-3-yl)-10,10-dimethyl-2¹,2²,2³,2⁶,6¹,6²,6³,6⁴,6⁵6⁶-decahydro-1¹H-8-oxa-1(5,3)-indola-6(1,3)-pyridazina-2(5,1)-pyridinacycloundecaphane-5,7-dione

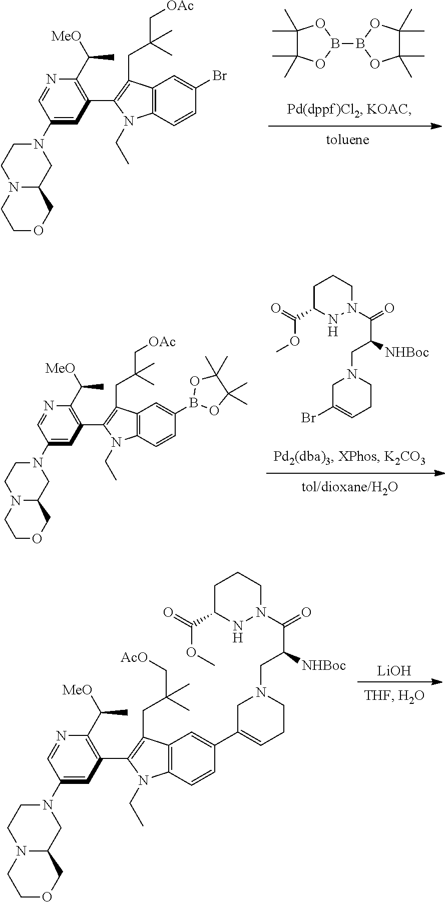

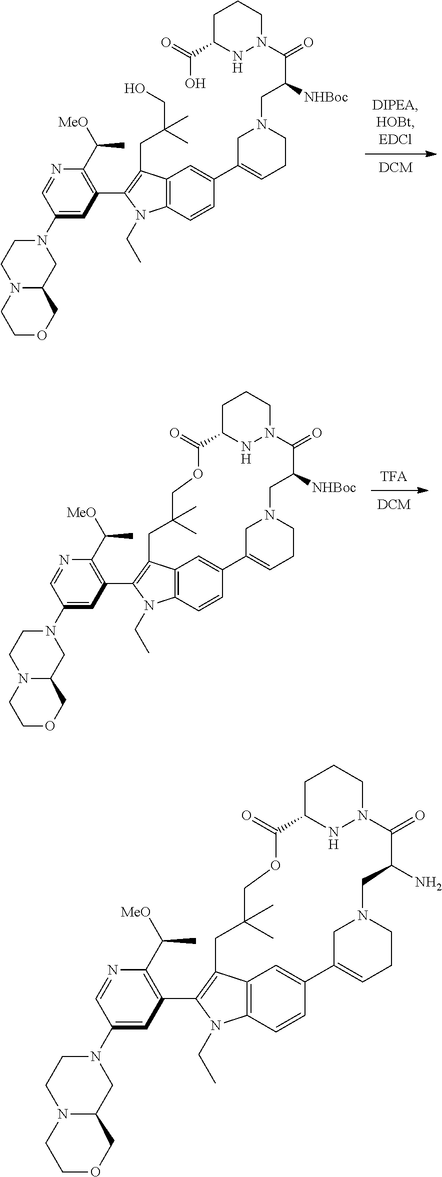

### Step 1: Synthesis of 3-(1-ethyl-2-(5-((S)-hexahydropyrazino[2,1-c][1,4]oxazin-8(1H)-yl)-2-((S)-1-methoxyethyl)pyridin-3-yl)-5-(4,4,5,5-tetramethyl-1,3,2-dioxaborolan-2-yl)-1H-indol-3-yl)-2,2-dimethylpropyl acetate

To a stirred solution of 3-(5-bromo-1-ethyl-2-(5-((S)-hexahydropyrazino[2,1-c][1,4]oxazin-8(1H)-yl)-2-((S)-1-methoxyethyl)pyridin-3-yl)-1H-indol-3-yl)-2,2-dimethylpropyl acetate (2.16 g, 3.45 mmol) in toluene (40 mL) was added KOAc (0.78 g, 7.967 mmol), 4,4,4′,4′,5,5,5′,5′-octamethyl-2,2′-bi(1,3,2-dioxaborolane (1.314 g, 5.175 mmol) and Pd(dppf)Cl₂ (0.23 g, 0.319 mmol, 0). The resulting mixture was stirred for 2 h at 90° C. under a nitrogen atmosphere. The mixture was basified to pH 8 with sat. aq. NaHCO₃. The resulting mixture was extracted with DCM (3×100 mL) and the combined organic layers were washed with brine (3×40 mL) and dried over anhydrous Na₂SO₄. After filtration, the filtrate was concentrated under reduced pressure. The residue was purified by silica gel column chromatography (2% MeOH/DCM) to afford the product (2 g, 86% yield) as a solid. LCMS (ESI) m/z [M+H] calcd for C₃₈H₅₅BN₄O₆: 675.43; found: 675.5

### Step 2: Synthesis of methyl (S)-1-((S)-3-(5-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)-3,6-dihydropyridin-1(2H)-yl)-2-((tert-butoxycarbonyl)amino)propanoyl)hexahydropyridazine-3-carboxylate

To a stirred solution of 3-(1-ethyl-2-(5-((S)-hexahydropyrazino[2,1-c][1,4]oxazin-8(1H)-yl)-2-((S)-1-methoxyethyl)pyridin-3-yl)-5-(4,4,5,5-tetramethyl-1,3,2-dioxaborolan-2-yl)-1H-indol-3-yl)-2,2-dimethylpropyl acetate (2 g, 2.964 mmol) and methyl (S)-1-((S)-3-(5-bromo-3,6-dihydropyridin-1(2H)-yl)-2-((tert-butoxycarbonyl)amino)propanoyl)hexahydropyridazine-3-carboxylate (2.25 g, 4.742 mmol) in toluene (12.5 mL), dioxane (8.3 mL), and H₂O (4.1 mL) was added K₂CO₃ (1.02 g, 7.410 mmol), X-Phos (0.57 g, 1.186 mmol), and Pd₂(dba)₃ (0.81 g, 0.889 mmol). The resulting mixture was stirred for 2 h at 70° C. under a nitrogen atmosphere. The mixture was basified to pH 8 with sat. aq. NaHCO₃. The resulting mixture was extracted with DCM (3×100 mL). and the combined organic layers were washed with brine (3×100 mL) and dried over anhydrous Na₂SO₄. After filtration, the filtrate was concentrated under reduced pressure. The residue was purified by silica gel column chromatography (2% MeOH/DCM) to afford the product (1.7 g, 55% yield) as a solid. LCMS (ESI) m/z [M+H] calcd for C₅₁H₇₄N₈O₉: 943.57; found: 943.7

### Step 3: Synthesis of (S)-1-((S)-2-((tert-butoxycarbonyl)amino)-3-(5-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)-3,6-dihydropyridin-1(2H)-yl)propanoyl)hexahydropyridazine-3-carboxylic acid

To a stirred solution of methyl (S)-1-((S)-3-(5-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)-3,6-dihydropyridin-1(2H)-yl)-2-((tert-butoxycarbonyl)amino)propanoyl)hexahydropyridazine-3-carboxylate (1.7 g, 1.802 mmol) in THE (9 mL) and H₂O (9 mL) was added LiOH (0.19 g, 8.109 mmol) at 0° C. The resulting mixture was stirred for 2 h at 0° C. The mixture was acidified to pH 6 with conc. HCl. The mixture was then extracted with DCM (3×50 mL) and dried over anhydrous Na₂SO₄. After filtration, the filtrate was concentrated under reduced pressure to afford the crude product (1.2 g, 67% yield) as a solid, which was used in the next step directly without further purification. LCMS (ESI) m/z [M+H] calcd for C₄₈H₇₀N₈O₈: 887.54; found: 887.6

### Step 4: Synthesis of tert-butyl ((63S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate

To a stirred solution of (S)-1-((S)-2-((tert-butoxycarbonyl)amino)-3-(5-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)-3,6-dihydropyridin-1(2H)-yl)propanoyl)hexahydropyridazine-3-carboxylic acid (1.2 g, 1.353 mmol) and HOBt (0.91 g, 6.765 mmol) in DCM (120 mL) was added EDC⋅HCl (7.26 g, 37.884 mmol) and DIPEA (6.12 g, 47.355 mmol) dropwise at 0° C. under a nitrogen atmosphere. The resulting mixture was stirred for 2 h at 0° C. The mixture was basified to pH 8 with sat. aq. NaHCO₃. The resulting mixture was extracted with DCM (3×100 mL). The combined organic layers were washed with brine (3×100 mL) and dried over anhydrous Na₂SO₄. After filtration, the filtrate was concentrated under reduced pressure. The residue was purified by silica gel column chromatography (3% MeOH/DCM) to afford the product (880 mg, 67% yield) as a solid. LCMS (ESI) m/z [M+H] calcd for C₄₈H₆₈N₈O₇: 869.53; found: 869.4

### Step 5: Synthesis of (6³S,4S)-4-amino-1¹-ethyl-12-(5-((S)-hexahydropyrazino[2,1-c][1,4]oxazin-8(1H)-yl)-2-((S)-1-methoxyethyl)pyridin-3-yl)-10,10-dimethyl-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione

To a stirred solution of tert-butyl ((63S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)carbamate (880 mg, 1.013 mmol) in DCM (8 mL) was added TFA (8 mL) dropwise at 0° C. The resulting mixture was stirred for 1 h at 0° C. The mixture was basified to pH 8 with sat. aq. NaHCO₃. The resulting mixture was extracted with DCM (3×50 mL) and the combined organic layers were washed with brine (3×50 mL) and dried over anhydrous Na₂SO₄. After filtration, the filtrate was concentrated under reduced pressure to afford the crude product (720 mg, 83% yield) as a solid, which was used in the next step directly without further purification. LCMS (ESI) m/z [M+H] calcd for C₄₃H₆₀N₈O₅: 769.48; found: 769.6

### Intermediate F25: Synthesis of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione

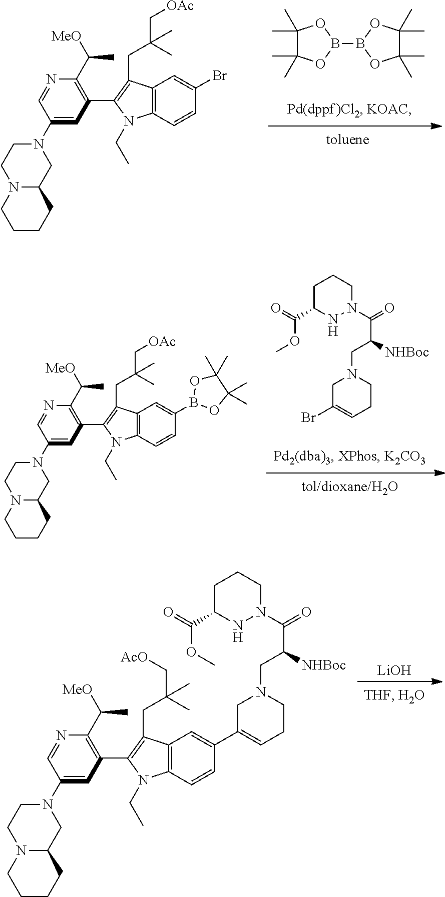

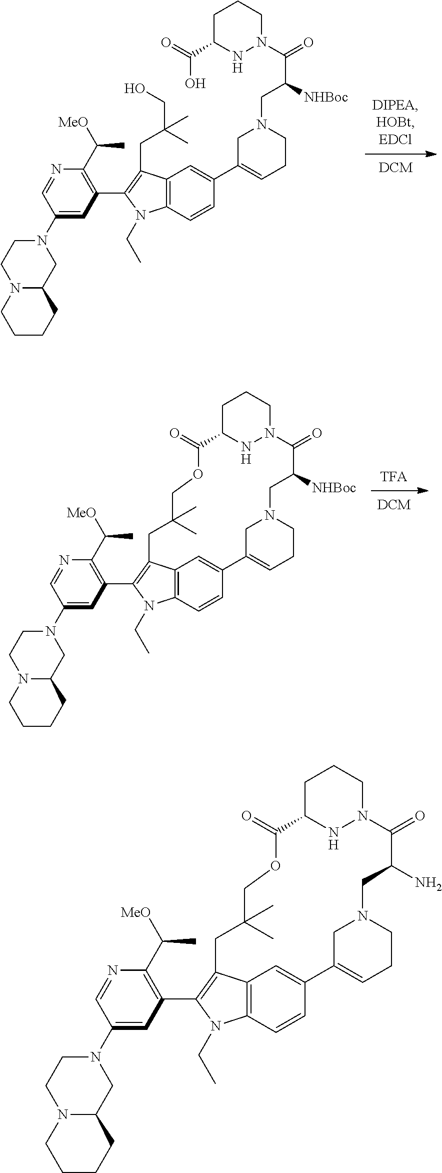

### Step 1: Synthesis of 3-(1-ethyl-2-(2-((S)-1-methoxyethyl)-5-((R)-octahydro-2H-pyrido[1,2-a]pyrazin-2-yl)pyridin-3-yl)-5-(4,4,5,5-tetramethyl-1,3,2-dioxaborolan-2-yl)-1H-indol-3-yl)-2,2-dimethylpropyl acetate

To a stirred solution of 3-(5-bromo-1-ethyl-2-(2-((S)-1-methoxyethyl)-5-((R)-octahydro-2H-pyrido[1,2-a]pyrazin-2-yl)pyridin-3-yl)-1H-indol-3-yl)-2,2-dimethylpropyl acetate (1 g, 1.598 mmol) and B₂Pin₂ (0.81 g, 3.196 mmol) in toluene (20 mL) was added KOAc (0.39 g, 3.995 mmol) and Pd(dppf)Cl₂ (0.12 g, 0.16 mmol). The mixture was stirred for 2 h at 90° C. under a nitrogen atmosphere. The mixture was then basified to pH 8 with sat. aq. NaHCO₃. The resulting mixture was extracted with DCM (3×40 mL) and the combined organic layers were washed with brine (3×40 mL) and dried over anhydrous Na₂SO₄. After filtration, the filtrate was concentrated under reduced pressure. The residue was purified by silica gel column chromatography (2% MeOH/DCM) to afford the product (0.9 g, 83% yield) as a solid.

LCMS (ESI) m/z [M+H] calcd for C₃₉H₅₇BN₄O₅: 673.45; found: 673.6

### Step 2: Synthesis of methyl (S)-1-((S)-3-(5-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)-3,6-dihydropyridin-1(2H)-yl)-2-((tert-butoxycarbonyl)amino)propanoyl)hexahydropyridazine-3-carboxylate

To a stirred solution of 3-(1-ethyl-2-(2-((S)-1-methoxyethyl)-5-((R)-octahydro-2H-pyrido[1,2-a]pyrazin-2-yl)pyridin-3-yl)-5-(4,4,5,5-tetramethyl-1,3,2-dioxaborolan-2-yl)-1H-indol-3-yl)-2,2-dimethylpropyl acetate (0.9 g, 1.338 mmol), methyl (3S)-1-[(2S)-3-(3-bromo-5,6-dihydro-2H-pyridin-1-yl)-2-[(tert-butoxycarbonyl)amino]propanoyl]-1,2-diazinane-3-carboxylate (1.02 g, 2.141 mmol), K₂CO₃ (0.46 g, 3.345 mmol), and X-Phos (0.26 g, 0.535 mmol) in toluene (13.5 mL), dioxane (90 mL), and H₂O (4.5 mL) was added Pd₂(dba)₃ (0.37 g, 0.401 mmol). The mixture was stirred for 2 h at 70° C. under a nitrogen atmosphere. The mixture was then basified to pH 8 with sat. aq. NaHCO₃. The resulting mixture was extracted with DCM (3×100 mL) and the combined organic layers were washed with brine (3×100 mL) and dried over anhydrous Na₂SO₄. After filtration, the filtrate was concentrated under reduced pressure. The residue was purified by silica gel column chromatography (2% MeOH/DCM) to afford the product (1.1 g, 87% yield) as a solid. LCMS (ESI) m/z [M+H] calcd for C₅₂H₇₆N₈O₈: 941.59; found: 941.8

### Step 3: Synthesis of (S)-1-((S)-2-((tert-butoxycarbonyl)amino)-3-(5-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)-3,6-dihydropyridin-1(2H)-yl)propanoyl)hexahydropyridazine-3-carboxylic acid

To a stirred solution of methyl (S)-1-((S)-3-(5-(3-(3-acetoxy-2,2-dimethylpropyl)-1-ethyl-2-(2-((S)-1-methoxyethyl)-5-((R)-octahydro-2H-pyrido[1,2-a]pyrazin-2-yl)pyridin-3-yl)-1H-indol-5-yl)-3,6-dihydropyridin-1(2H)-yl)-2-((tert-butoxycarbonyl)amino)propanoyl)hexahydropyridazine-3-carboxylate (1.1 g, 1.169 mmol) in THE (8 mL) was added a solution of LiOH (0.14 g, 5.845 mmol) in H₂O (8 mL) dropwise at 0° C. under a nitrogen atmosphere. The reaction mixture was stirred for 16 h. The mixture was then acidified to pH 6 with conc. HCl. The resulting mixture was extracted with DCM (3×50 mL) and dried over anhydrous Na₂SO₄. After filtration, the filtrate was concentrated under reduced pressure to afford the product (1.0 g, 96% yield) as a solid, which was used in the next step directly without further purification.

LCMS (ESI) m/z [M+H] calcd for C₄₉H₇₂N₈O₇: 885.56; found: 885.5

### Step 4: Synthesis of tert-butyl ((63S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹ 2²,2³,2⁶,6¹¹,6²,6³,6⁴,6⁵, 6⁶-decahydro-1¹H-8-oxa-1 (5,3)-indola-6(1,3)-pyridazina-2(5,1)-pyridinacycloundecaphane-4-yl)carbamate

To a stirred solution of (S)-1-((S)-2-((tert-butoxycarbonyl)amino)-3-(5-(1-ethyl-3-(3-hydroxy-2,2-dimethylpropyl)-2-(2-((S)-1-methoxyethyl)-5-((R)-octahydro-2H-pyrido[1,2-a]pyrazin-2-yl)pyridin-3-yl)-1H-indol-5-yl)-3,6-dihydropyridin-1(2H)-yl)propanoyl)hexahydropyridazine-3-carboxylic acid (1.0 g, 1.13 mmol) and HOBt (0.76 g, 5.65 mmol) in DCM (100 mL) was added EDC⋅HCl (6.06 g, 31.64 mmol) and DIPEA (5.11 g, 39.55 mmol) dropwise at 0° C. under a nitrogen atmosphere. The reaction mixture was stirred for 16 h. The mixture was then basified to pH 8 with sat. aq. NaHCO₃. The resulting mixture was extracted with DCM (3×100 mL) and the combined organic layers were washed with brine (3×100 mL) and dried over anhydrous Na₂SO₄. After filtration, the filtrate was concentrated under reduced pressure. The residue was purified by silica gel column chromatography (3% MeOH/DCM) to afford the product (650 mg, 66% yield) as a solid. LCMS (ESI) m/z [M+H] calcd for C₄₉H₇₀N₈O₆: 867.55; found: 867.5

### Step 5: Synthesis of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-2¹ 2²,2³,2⁶,6¹,6²,6³,6⁴,6⁵, 6⁶-decahydro-1¹H-8-oxa-1(5,3)-indola-6(1,3)-pyridazina-2(5,1)-pyridinacycloundecaphane-5,7-dione

To a stirred solution of tert-butyl ((63S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate (300 mg, 0.346 mmol) in DCM (3 mL) was added TFA (3 mL) dropwise at 0° C. under a nitrogen atmosphere. The resulting mixture was stirred for 1 h at 0° C. The mixture was then basified to pH 8 with sat. aq. NaHCO₃. The resulting mixture was extracted with DCM (3×50 mL) and the combined organic layers were washed with brine (3×50 mL) and dried over anhydrous Na₂SO₄. After filtration, the filtrate was concentrated under reduced pressure to afford the product (260 mg, 98% yield) as a solid, which was used in the next step directly without further purification. LCMS (ESI) m/z [M+H] calcd for C₄₄H₆₂N₈O₄: 767.50; found: 767.2

### Intermediate F26: Synthesis of (2²S,6³S,4S)-4-amino-1²-(5-(4-cyclopropylpiperazin-1-yl)-2-((S)-1-methoxyethyl)pyridin-3-yl)-1¹-ethyl-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

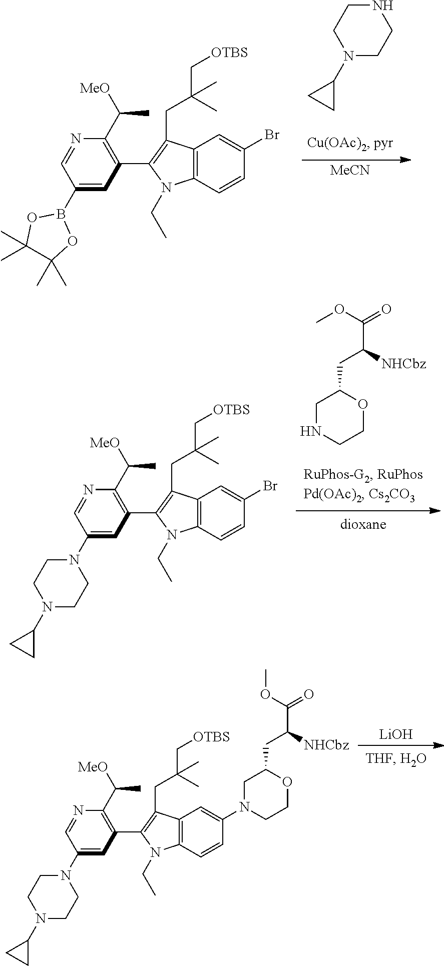

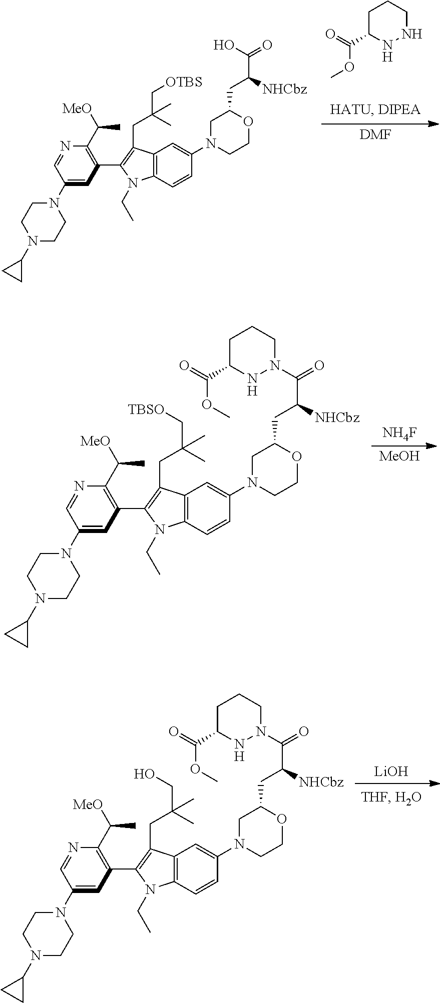

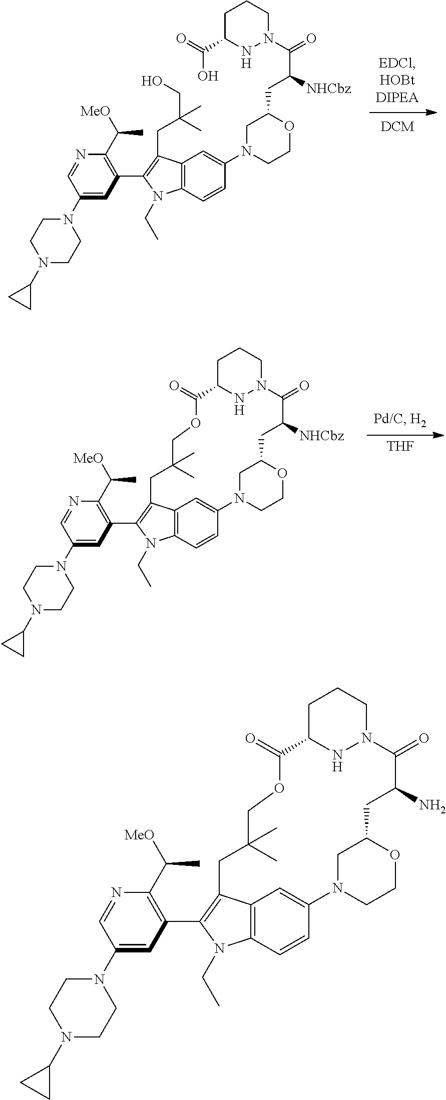

### Step 1: Synthesis of (S)-5-bromo-3-(3-((tert-butyldimethylsilyl)oxy)-2,2-dimethylpropyl)-2-(5-(4-cyclopropylpiperazin-1-yl)-2-(1-methoxyethyl)pyridin-3-yl)-1-ethyl-1H-indole

To a solution of (S)-5-bromo-3-(3-((tert-butyldimethylsilyl)oxy)-2,2-dimethylpropyl)-1-ethyl-2-(2-(1-methoxyethyl)-5-(4,4,5,5-tetramethyl-1,3,2-dioxaborolan-2-yl)pyridin-3-yl)-1H-indole (20 g, 29.2 mmol) in MeCN (100 mL) was added 1-cyclopropylpiperazine (5.53 g, 43.8 mmol), pyridine (6.93 g, 87.6 mmol) and Cu(OAC)₂ (10.61 g, 58.4 mmol), followed by the addition of 4 Å MS (20 g). The reaction was stirred at 60° C. for 16 h under O₂. The mixture was then filtered and concentrated under reduced pressure. The residue was purified by silica gel column chromatography (EtOAc/Pet. ether 2:1 then EtOAc/MeOH 10:1) to afford the desired product (6 g, 30% yield) as a solid. LCMS (ESI) m/z [M+H] calcd for C₃₆H₅₅BrN₄O₂Si: 683.34; found: 683.3.

### Step 2: Synthesis of methyl (S)-2-(((benzyloxy)carbonyl)amino)-3-((S)-4-(3-(3-((tert-butyldimethylsilyl)oxy)-2,2-dimethylpropyl)-2-(5-(4-cyclopropylpiperazin-1-yl)-2-((S)-1-methoxyethyl)pyridin-3-yl)-1-ethyl-1H-indol-5-yl)morpholin-2-yl)propanoate

To a solution of (S)-5-bromo-3-(3-((tert-butyldimethylsilyl)oxy)-2,2-dimethylpropyl)-2-(5-(4-cyclopropylpiperazin-1-yl)-2-(1-methoxyethyl)pyridin-3-yl)-1-ethyl-1H-indole (4 g, 5.8 mmol) in dioxane (40 mL) was added methyl (S)-2-(((benzyloxy)carbonyl)amino)-3-((S)-morpholin-2-yl)propanoate (2.8 g, 8.7 mmol), RuPhos (0.81 g, 1.7 mmol), Cs₂CO₃ (5.67 g, 17.4 mmol), Ruphos Pd G2 (0.45 g, 0.6 mmol) and Pd(OAc)₂ (0.13 g, 0.6 mmol). The reaction was stirred at 105° C. for 4 h under N₂. The mixture was concentrated under reduced pressure and the residue was purified by column chromatography (basic Al₂O₃, EtOAc/pet. ether 1:1) to afford the desired product (2 g, 38% yield) as a solid. LCMS (ESI) m/z [M+H] calcd for C₅₂H₇₆N₆O₇Si: 925.56; found: 925.5.

### Step 3: Synthesis of (S)-2-(((benzyloxy)carbonyl)amino)-3-((S)-4-(3-(3-((tert-butyldimethylsilyl)oxy)-2,2-dimethylpropyl)-2-(5-(4-cyclopropylpiperazin-1-yl)-2-((S)-1-methoxyethyl)pyridin-3-yl)-1-ethyl-1H-indol-5-yl)morpholin-2-yl)propanoic acid

To a solution of methyl (S)-2-(((benzyloxy)carbonyl)amino)-3-((S)-4-(3-(3-((tert-butyldimethylsilyl)oxy)-2,2-dimethylpropyl)-2-(5-(4-cyclopropylpiperazin-1-yl)-2-((S)-1-methoxyethyl)pyridin-3-yl)-1-ethyl-1H-indol-5-yl)morpholin-2-yl)propanoate (2 g, 2.2 mmol) in THE (20 mL) and H₂O (6 mL) was added LiOH (0.26 g. 11 mmol) at 0° C. The reaction was stirred at room temperature for 3 h. The mixture was adjusted to pH 6 with 1 N HCl and extracted with EtOAc (2×30 mL). The combined organic layers were washed with brine, dried over Na₂SO₄, filtered and concentrated under reduced pressure to afford (1.8 g crude) as a solid, which was used in the next step directly without further purification. LCMS (ESI) m/z [M+H] calcd for C₅₁H₇₄N₆O₇Si: 911.55; found: 911.5.

### Step 4: Synthesis of methyl (S)-1-((S)-2-(((benzyloxy)carbonyl)amino)-3-((S)-4-(3-(3-((tert-butyldimethylsilyl)oxy)-2,2-dimethylpropyl)-2-(5-(4-cyclopropylpiperazin-1-yl)-2-((S)-1-methoxyethyl)pyridin-3-yl)-1-ethyl-1H-indol-5-yl)morpholin-2-yl)propanoyl)hexahydropyridazine-3-carboxylate

To a solution of (S)-2-(((benzyloxy)carbonyl)amino)-3-((S)-4-(3-(3-((tert-butyldimethylsilyl)oxy)-2,2-dimethylpropyl)-2-(5-(4-cyclopropylpiperazin-1-yl)-2-((S)-1-methoxyethyl)pyridin-3-yl)-1-ethyl-1H-indol-5-yl)morpholin-2-yl)propanoic acid (1.8 g, 2.0 mmol) in DMF (15 mL) was added a solution of methyl (3S)-1,2-diazinane-3-carboxylate (0.43 g, 3 mmol) and DIPEA (1.3 g, 10 mmol) at 0° C. in DMF (5 mL), followed by HATU (0.91 g, 2.4 mmol). The reaction was stirred at 0° C. for 2 h. The mixture was diluted with EtOAc (40 mL) and quenched with H₂O (30 mL). The organic layer was washed with H₂O (2×30 mL) and brine (30 mL), dried over Na₂SO₄, filtered, and concentrated under reduced pressure. The residue was purified by normal phase column chromatography (EtOAc/MeOH 20:1) to afford the desired product (1.8 g, 85% yield) as a solid. LCMS (ESI) m/z [M/2+H] calcd for C₅₇H₈₄N₈O₈Si: 519.32; found: 519.4.

### Step 5: Synthesis of methyl (S)-1-((S)-2-(((benzyloxy)carbonyl)amino)-3-((S)-4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)morpholin-2-yl)propanoyl)hexahydropyridazine-3-carboxylate

To a solution of methyl (S)-1-((S)-2-(((benzyloxy)carbonyl)amino)-3-((S)-4-(3-(3-((tert-butyldimethylsilyl)oxy)-2,2-dimethylpropyl)-2-(5-(4-cyclopropylpiperazin-1-yl)-2-((S)-1-methoxyethyl)pyridin-3-yl)-1-ethyl-1H-indol-5-yl)morpholin-2-yl)propanoyl)hexahydropyridazine-3-carboxylate (1.8 g, 1.7 mmol) in MeOH (20 mL) was added NH₄F (2.52 g, 67.9 mmol). The reaction was stirred at 80° C. for 16 h. The mixture was concentrated under reduced pressure, and the residue was diluted with DCM (30 mL). After filtration, the filtrate was concentrated under reduced pressure to afford the dired product (1.7 g crude) as a solid, which was used in the next step directly without further purification. LCMS (ESI) m/z [M+H] calcd for C₅₁H₇₀N₈O₈: 923.54; found: 923.4.

### Step 6: Synthesis of (S)-1-((S)-2-(((benzyloxy)carbonyl)amino)-3-((S)-4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)morpholin-2-yl)propanoyl)hexahydropyridazine-3-carboxylic acid

To a solution of methyl (S)-1-((S)-2-(((benzyloxy)carbonyl)amino)-3-((S)-4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)morpholin-2-yl)propanoyl)hexahydropyridazine-3-carboxylate (1.8 g, 1.9 mmol) in THE (20 mL) and H₂O (5 mL) was added LiOH (0.23 g, 9.5 mmol) at 0° C. The reaction was stirred at room temperature for 3 h. The mixture was adjusted to pH-7 with 1 N HCl. The resulting solution was concentrated under reduced pressure to afford the desired product (1.8 g crude) as a solid, which was used in the next step directly without further purification. LCMS (ESI) m/z [M+H] calcd for C₅₀H₆₈N₈O₈: 909.53; found: 909.5.

### Step 7: Synthesis of benzyl ((2²S,6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate

To a solution of (S)-1-((S)-2-(((benzyloxy)carbonyl)amino)-3-((S)-4-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)morpholin-2-yl)propanoyl)hexahydropyridazine-3-carboxylic acid (1.6 g, 1.8 mmol) in DCM (160 mL) was added DIPEA (6.98 g, 54 mmol), HOBt (2.43 g, 18 mmol) and EDCI (10.4 g, 54 mmol). The reaction was stirred at 30° C. for 16 h. The mixture was concentrated under reduced pressure, the residue was diluted with EtOAc (50 mL). The organic layer was washed with H₂O (2×40 mL) and brine (40 mL), dried over Na₂SO₄, filtered and concentrated under reduced pressure. The residue was purified by normal phase column chromatography (EtOAc/MeOH 10:1) to afford the desired product (0.8 g, 50% yield) as a solid.

LCMS (ESI) m/z [M+H] calcd for C₅₀H₆₆N₈O₇: 891.52; found: 891.6.

### Step 8: Synthesis of (2²S,6³S,4S)-4-amino-1²-(5-(4-cyclopropylpiperazin-1-yl)-2-((S)-1-methoxyethyl)pyridin-3-yl)-1¹-ethyl-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

To a solution of benzyl ((2²S,6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate (700 mg, 0.79 mmol) in THE (10 mL) was added 10% Pd/C (350 mg). The reaction was stirred for 6 h under H₂ (1 atm). The mixture was filtered and concentrated under reduced pressure. The residue was purified by normal phase column chromatography (EtOAc (1% NH₃H₂O)/MeOH (1% NH₃H₂O) 10:1) to afford the desired product (420 mg, 71% yield) as a solid. LCMS (ESI) m/z [M+H] calcd for C₄₂H₆₀N₈O₅: 757.48; found: 757.5.

### Intermediate F27: Synthesis of (6³S,4S)-4-amino-1²-(5-(4-cyclopropylpiperazin-1-yl)-2-((S)-1-methoxyethyl)pyridin-3-yl)-1¹-ethyl-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione

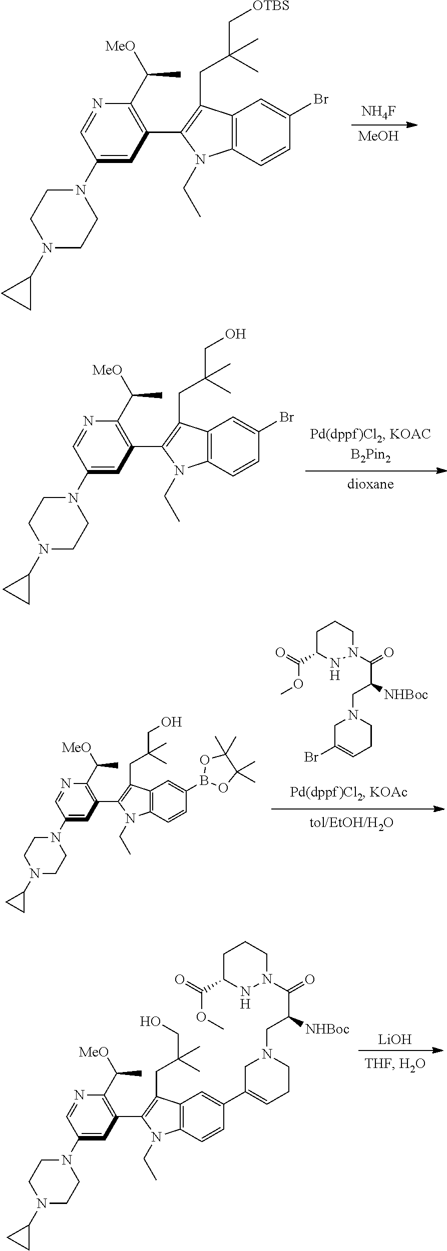

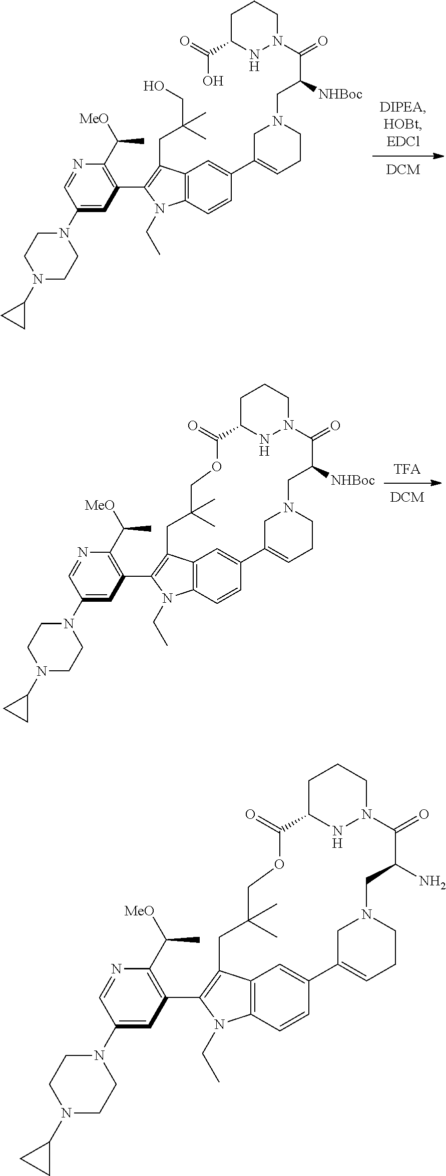

### Step 1: Synthesis of (S)-3-(5-bromo-2-(5-(4-cyclopropylpiperazin-1-yl)-2-(1-methoxyethyl)pyridin-3-yl)-1-ethyl-1H-indol-3-yl)-2,2-dimethylpropan-1-ol

A solution of (S)-5-bromo-3-(3-((tert-butyldimethylsilyl)oxy)-2,2-dimethylpropyl)-2-(5-(4-cyclopropylpiperazin-1-yl)-2-(1-methoxyethyl)pyridin-3-yl)-1-ethyl-1H-indole (3 g, 4 mmol) and NH₄F (6.5 g, 176 mmol, 40 eq) in MeOH (30 mL) was stirred for 16 h at 80° C. The reaction mixture was diluted with EtOAc (50 mL) and washed with H₂O (2×50 mL). The organic phase was concentrated under reduced pressure to afford (4.2 g, 95% yield) as a solid. LCMS (ESI) m/z [M+H] calcd for C₃₀H₄₁BrN₄O₂: 569.25; found: 569.3.

### Step 2: Synthesis of (S)-3-(2-(5-(4-cyclopropylpiperazin-1-yl)-2-(1-methoxyethyl)pyridin-3-yl)-1-ethyl-5-(4,4,5,5-tetramethyl-1,3,2-dioxaborolan-2-yl)-1H-indol-3-yl)-2,2-dimethylpropan-1-ol

To a solution of (S)-3-(5-bromo-2-(5-(4-cyclopropylpiperazin-1-yl)-2-(1-methoxyethyl)pyridin-3-yl)-1-ethyl-1H-indol-3-yl)-2,2-dimethylpropan-1-ol (3 g, 5.3 mmol) and 4,4,5,5-tetramethyl-2-(4,4,5,5-tetramethyl-1,3,2-dioxaborolan-2-yl)-1,3,2-dioxaborolane (1.5 g, 5.8 mmol) in 1,4-dioxane (30 mL) was added KOAc (1 g, 10.5 mmol) followed by Pd(dppf)Cl₂⋅DCM (860 mg, 1.1 mmol) under N₂ atmosphere. The resulting mixture was stirred for 6 hours at 85° C. The mixture was concentrated under pressure to give a residue. The residue was purified by normal phase column chromatography (EtOAc/pet. ether 5:1) to afford the desired product (2 g, 61% yield) as an oil. LCMS (ESI) m/z [M+H] calcd for C₃₆H₅₃BN₄O₄: 617.43; found: 617.3.

### Step 3: Synthesis of methyl (S)-1-((S)-2-((tert-butoxycarbonyl)amino)-3-(5-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-3,6-dihydropyridin-1(2H)-yl)propanoyl)hexahydropyridazine-3-carboxylate

To a solution of (S)-3-(2-(5-(4-cyclopropylpiperazin-1-yl)-2-(1-methoxyethyl)pyridin-3-yl)-1-ethyl-5-(4,4,5,5-tetramethyl-1,3,2-dioxaborolan-2-yl)-1H-indol-3-yl)-2,2-dimethylpropan-1-ol (1.7 g, 2.8 mmol) and methyl (S)-1-((S)-3-(5-bromo-3,6-dihydropyridin-1(2H)-yl)-2-((tert-butoxycarbonyl)amino)propanoyl)hexahydropyridazine-3-carboxylate (1.4 g, 3 mmol) in toluene (20 mL), EtOH (4 mL), and H₂O (1 mL) was added K₂CO₃ (1.2 g, 8.4 mmol) followed by Pd(dppf)Cl₂⋅DCM (230 mg, 0.28 mmol) under N₂ atmosphere. The resulting mixture was stirred for 6 hours at 85° C. under N₂ atmosphere. The mixture was concentrated under reduced pressure to give a residue. The residue was purified by normal phase column chromatography (EtOAc/pet. ether 5:1) to afford the desired product (1 g, 40% yield) as a solid. LCMS (ESI) m/z [M+H] calcd for C₄₉H₇₂N₈O₇: 885.56; found: 885.5.

### Step 4: Synthesis of (S)-1-((S)-2-((tert-butoxycarbonyl)amino)-3-(5-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-3,6-dihydropyridin-1(2H)-yl)propanoyl)hexahydropyridazine-3-carboxylic acid

To a solution of methyl (S)-1-((S)-2-((tert-butoxycarbonyl)amino)-3-(5-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-3,6-dihydropyridin-1(2H)-yl)propanoyl)hexahydropyridazine-3-carboxylate (900 mg, 1 mmol) in THE (4 mL) was added a solution of lithium hydroxide (260 mg, 6.1 mmol) in H₂O (1 mL). The resulting mixture was stirred for 3 hours. The reaction mixture was treated with 1 N HCl to pH to 4 at 0° C. The mixture was extracted with DCM (2×20 mL) and the organic layer was washed with brine. The solution was concentrated under reduced pressure to afford the desired product (1.0 g) as a solid, which was used directly in the next step. LCMS (ESI) m/z [M+H] calcd for C₄₈H₇₀N₈O₇: 871.55; found: 871.5.

### Step 5: Synthesis of tert-butyl ((63S,4S)-1²-(5-(4-cyclopropylpiperazin-1-yl)-2-((S)-1-methoxyethyl)pyridin-3-yl)-1¹-ethyl-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate

To a stirred solution of (S)-1-((S)-2-((tert-butoxycarbonyl)amino)-3-(5-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-3,6-dihydropyridin-1(2H)-yl)propanoyl)hexahydropyridazine-3-carboxylic acid (900 mg, 1 mmol) and DIPEA (4 g, 30 mmol) in DCM (100 mL) was added EDCI (5.9 g, 30 mmol) and HOBt (1.4 g, 10 mmol). The resulting mixture was stirred for 16 hours at 35° C. under an argon atmosphere. The resulting mixture was concentrated under reduced pressure and the residue was purified by normal phase column chromatography (pet. ether/EtOAc/NH₃⋅H₂O(1:5:0.05) to afford the desired product (450 mg, 53% yield) as a solid. LCMS (ESI) m/z [M+H] calcd for C₄₈H₆₈N₈O₆: 853.54; found: 853.4.

### Step 6: Synthesis of (6³S,4S)-4-amino-1²-(5-(4-cyclopropylpiperazin-1-yl)-2-((S)-1-methoxyethyl)pyridin-3-yl)-1¹-ethyl-10,10-dimethyl-2¹ 2²,2³,2⁶,6¹,6²,6³,6⁴,6⁵,6⁶-decahydro-1′H-8-oxa-1(5,3)-indola-6(1,3)-pyridazina-2(5,1)-pyridinacycloundecaphane-5,7-dione

To a solution of tert-butyl ((6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate (230 mg, 0.024 mmol) in DCM (1.5 mL) was added TFA (0.5 mL). The solution was stirred for 1 h and was then concentrated under reduced pressure to afford the desired product (280 mg). LCMS (ESI) m/z [M+H] calcd for C₄₃H₆₀N₈O₄: 753.48; found: 753.5.

### Intermediate F28: Synthesis of (6³S,4S,Z)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

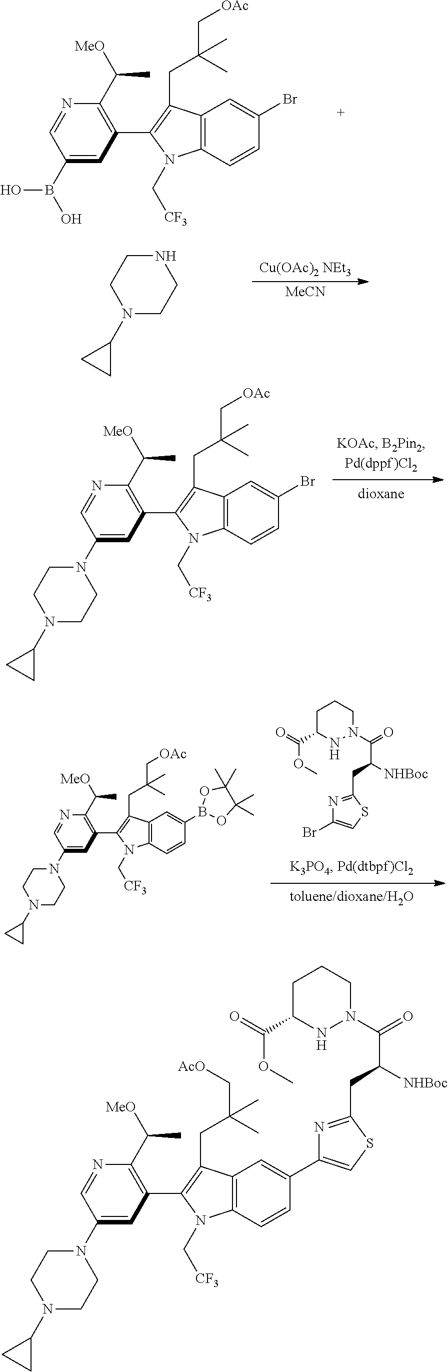

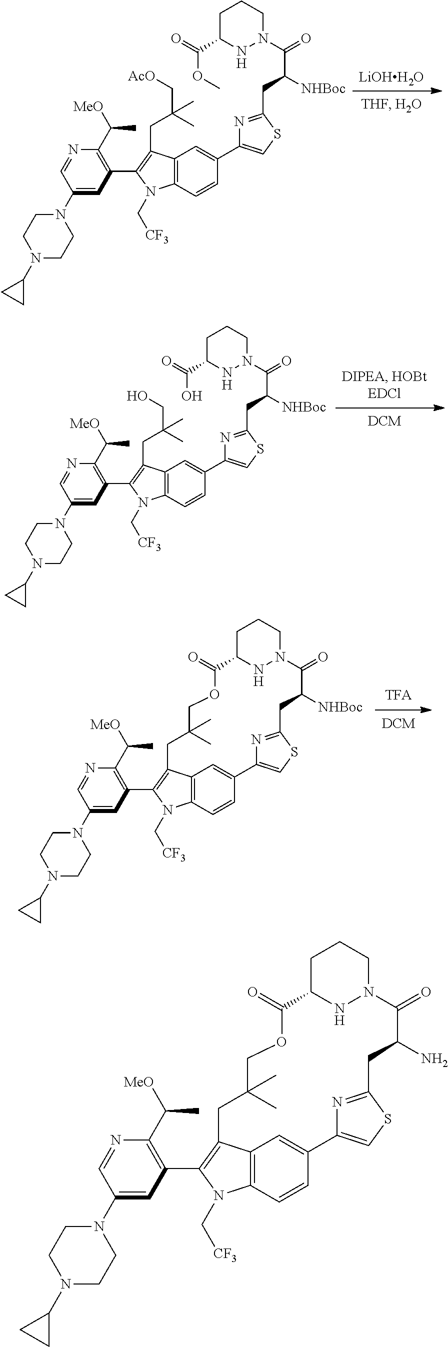

### Step 1: Synthesis of (S)-3-(5-bromo-2-(5-(4-cyclopropylpiperazin-1-yl)-2-(1-methoxyethyl)pyridin-3-yl)-1-(2,2,2-trifluoroethyl)-1H-indol-3-yl)-2,2-dimethylpropyl acetate

To a stirred solution of (S)-(5-(3-(3-acetoxy-2,2-dimethylpropyl)-5-bromo-1-(2,2,2-trifluoroethyl)-1H-indol-2-yl)-6-(1-methoxyethyl)pyridin-3-yl)boronic acid (40 g, 61.514 mmol) and NEt₃ (12.45 g, 123.028 mmol) in MeCN (1000 mL) was added 4 Å MS (8 g) and 1-cyclopropylpiperazine (38.82 g, 307.570 mmol) in portions under an oxygen atmosphere. The resulting mixture was stirred for 1 h at room temperature under an oxygen atmosphere. To the above mixture was added Cu(OAc)₂ (22.35 g, 123.028 mmol) and then the vessel was evacuated, backfilled with oxygen, and then stirred overnight at room temperature. The resulting mixture was filtered and was concentrated under reduced pressure. The residue was diluted with EtOAc (300 mL) and the organic layer was washed with NH₃⋅H₂O (4×100 mL), dried over Na₂SO₄, and concentrated under reduce pressure. The residue was purified by column chromatography (50% EtOAc/pet. ether) to afford the desired product (23.7 g, 56% yield) as a solid.

LCMS (ESI) m/z [M+H] calcd for C₃₂H₄₀BrF₃N₄O₃ 665.23; found: 666.0.

### Step 2: Synthesis of (S)-3-(2-(5-(4-cyclopropylpiperazin-1-yl)-2-(1-methoxyethyl)pyridin-3-yl)-5-(4,4,5,5-tetramethyl-1,3,2-dioxaborolan-2-yl)-1-(2,2,2-trifluoroethyl)-1H-indol-3-yl)-2,2-dimethylpropyl acetate

To a solution of (S)-3-(5-bromo-2-(5-(4-cyclopropylpiperazin-1-yl)-2-(1-methoxyethyl)pyridin-3-yl)-1-(2,2,2-trifluoroethyl)-1H-indol-3-yl)-2,2-dimethylpropyl acetate (26 g, 39.063 mmol) and KOAc (13.42 g, 136.721 mmol) in dioxane (260 mL) was added B₂Pin₂ (37.7 g, 148.4 mmol) and Pd(dppf)Cl₂ (2.86 g, 3.906 mmol). The resulting mixture was evacuated and backfilled with argon then stirred at 90° C. for 3 h. The resulting mixture was filtered, the filter cake was washed with EtOAc (2×200 mL), and the filtrate was concentrated under reduced pressure. The residue was purified by silica gel column chromatography, (80% EtOAc/pet. ether) to afford the desired product (17 g, 61% yield) as a solid. LCMS (ESI) m/z [M+H] calcd for C₃₈H₅₂BF₃N₄O₅ 713.41; found: 713.3.

### Step 3: Synthesis of methyl (S)-1-((S)-3-(4-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)thiazol-2-yl)-2-((tert-butoxycarbonyl)amino)propanoyl)hexahydropyridazine-3-carboxylate

To a solution of (S)-3-(2-(5-(4-cyclopropylpiperazin-1-yl)-2-(1-methoxyethyl)pyridin-3-yl)-5-(4,4,5,5-tetramethyl-1,3,2-dioxaborolan-2-yl)-1-(2,2,2-trifluoroethyl)-1H-indol-3-yl)-2,2-dimethylpropyl acetate (17 g, 23.854 mmol), methyl (S)-1-((S)-3-(4-bromothiazol-2-yl)-2-((tert-butoxycarbonyl)amino)propanoyl)hexahydropyridazine-3-carboxylate (13.66 g, 28.625 mmol) and K₃PO₄ (12.66 g, 59.635 mmol) in toluene (170 mL), dioxane (57 mL) and H₂O (57 mL) was added 1,1′-bis(di-tert-butylphosphino) ferrocene palladium dichloride (1.55 g, 2.385 mmol) in portions under an argon atmosphere. To the mixture was added. The resulting mixture was stirred at 70° C. for 2 h. The mixture was filtered, the filter cake was washed with EtOAc (3×100 mL). The aqueous layer was extracted with EtOAc (3×200 mL), and the combined organic layers were washed with brine (2×200 mL), dried over Na₂SO₄, filtered, and concentrated under reduced pressure. The residue was purified by silica gel column chromatography (3% MeOH/DCM) to afford the desired product (20.4 g, 87% yield) as a solid. LCMS (ESI) m/z [M+H] calcd for C₄₉H₆₅F₃N₈O₈S 983.47; found: 983.6.

### Step 4: Synthesis of (S)-1-((S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)thiazol-2-yl)propanoyl)hexahydropyridazine-3-carboxylic acid

To a solution of methyl (S)-1-((S)-3-(4-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)thiazol-2-yl)-2-((tert-butoxycarbonyl)amino)propanoyl)hexahydropyridazine-3-carboxylate (20 g, 20.343 mmol) in THE (200 mL) was added a solution of LiOH (2.56 g, 61.029 mmol) in H₂O (61 mL) at 0° C. The resulting mixture was stirred overnight at room temperature. The mixture was then acidified to pH 6 with 1 N HCl (aq.) and was then extracted with EtOAc (3×100 mL). The combined organic layers were washed with brine (2×100 mL), dried over Na₂SO₄, filtered, and concentrated under reduced pressure to afford the crude product (18.8 g), which was used directly in the next step without purification. LCMS (ESI) m/z [M+H] calcd for C₄₆H₆₁F₃N₈O₇S 927.44; found: 927.3.

### Step 5: Synthesis of tert-butyl ((6³S,4S,Z)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

To a solution of (S)-1-((S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)thiazol-2-yl)propanoyl)hexahydropyridazine-3-carboxylic acid (15 g, 16.179 mmol), DIPEA (112.73 mL, 647.160 mmol), and HOBt (43.72 g, 323.580 mmol) in DCM (768 mL) at 0° C. was added EDCI (93.05 g, 485.370 mmol). The resulting mixture was stirred overnight at room temperature. The reaction mixture was quenched by the addition of cold H₂O (500 mL). The resulting mixture was extracted with EtOAc (3×500 mL), and the combined organic layers were washed with brine (2×500 mL), dried over Na₂SO₄, filtered, and concentrated under reduced pressure. The residue was purified by silica gel column chromatography (3% MeOH/DCM) to afford the desired product (7.5 g, 51% yield) as a solid. LCMS (ESI) m/z [M+H] calcd for C₄₆H₅₉F₃N₈O₆S 909.43; found: 909.3.

### Step 6: Synthesis of (6³S,4S,Z)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

To a solution of tert-butyl ((63S,4S,2)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (8.2 g, 9.02 mmol) in DCM (80 mL) at 0° C. was added TFA (40 mL, 538.52 mmol). The resulting mixture was stirred for 2 h at room temperature. The mixture was then concentrated under reduced pressure and the residue was adjusted to pH 8 with sat. NaHCO₃ (aq.). The resulting mixture was extracted with EtOAc (3×300 mL), and the combined organic layers were washed with brine (300 mL), dried over Na₂SO₄, filtered, and concentrated under reduced pressure to afford the desired product (8.0 g, 98% yield) as a solid.

LCMS (ESI) m/z [M+H] calcd for C₄₁H₅₁F₃N₈O₄S 809.38; found: 809.5.

### Intermediate F29: Synthesis of (2²S,6³S,4S)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-11H-8-oxa-2(4,2)-morpholina-1(5,3)-indola-6(1,3)-pyridazinacycloundecaphane-5,7-dione

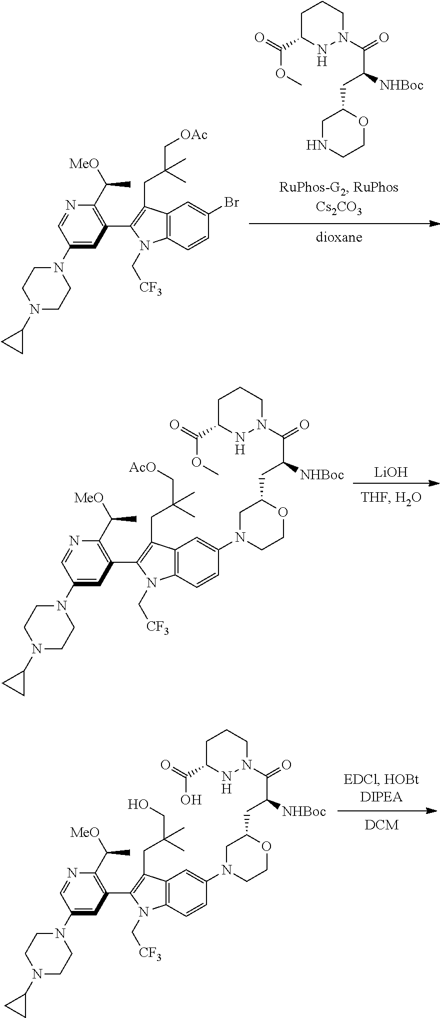

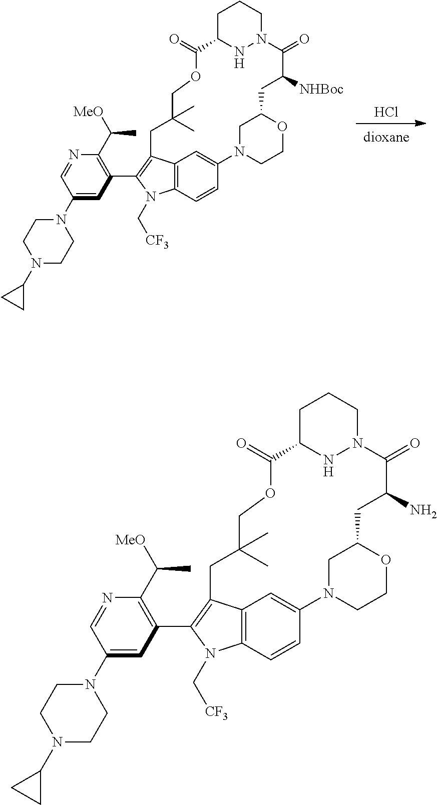

### Step 1: Synthesis of methyl (S)-1-((S)-3-((S)-4-(3-(3-acetoxy-2,2-dimethyl propyl)-2-(5-(4-cyclopropylpiperazin-1-yl)-2-((S)-1-methoxyethyl)pyridin-3-yl)-1-(2,2,2-trifluoroethyl)-1H-indol-5-yl)morpholin-2-yl)-2-((tert-butoxycarbonyl)amino)propanoyl)hexahydropyridazine-3-carboxylate

To a solution of 3(S)-3-(5-bromo-2-(5-(4-cyclopropylpiperazin-1-yl)-2-(1-methoxyethyl)pyridin-3-yl)-1-(2,2,2-trifluoroethyl)-1H-indol-3-yl)-2,2-dimethylpropyl acetate (65.0 g, 97.66 mmol) and methyl (S)-1-((S)-2-((tert-butoxycarbonyl)amino)-3-((S)-morpholin-2-yl)propanoyl)hexahydropyridazine-3-carboxylate (78.2 g, 0.195 mol) in dioxane (650 mL) was added RuPhos (27.3 g, 58.60 mmol), RuPhos-G2-Pd (22.7 g, 29.30 mmol), and Cs₂CO₃ (95.5 g, 0.29 mol). The resulting mixture was stirred overnight at 80° C. The reaction mixture was then filtered, the filter cake was washed with EtOAc (3×300 mL), and the filtrate was concentrated under reduced pressure. The residue was purified by silica gel column chromatography to afford the desired product (63 g, 65% yield) as a solid. LCMS (ESI) m/z [M+H] calcd for C₅₀H₇₁F₃NaO₉ 985.54, found: 985.8.

### Step 2: Synthesis of (S)-1-((S)-2-((tert-butoxycarbonyl)amino)-3-((S)-4-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)morpholin-2-yl)propanoyl)hexahydropyridazine-3-carboxylic acid

To a solution of methyl (S)-1-((S)-3-((S)-4-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)morpholin-2-yl)-2-((tert-butoxycarbonyl)amino)propanoyl)hexahydropyridazine-3-carboxylate (79 g, 80.19 mmol) in THF (700 mL) was added a solution of LiOH⋅H₂O (16.7 g, 0.398 mol) in H₂O (150 mL) at 0° C. The resulting mixture was stirred for 5 h at room temperature. The mixture was then acidified to pH 5 with 1M HCl. The aqueous layer was extracted with DCM (3×500 mL) and the organic layer was dried over Na₂SO₄, filtered, and concentrated under reduced pressure to afford the desired product (70 g, crude) as a solid. LCMS (ESI) m/z [M+H] calcd for C₄₇H₆₇F₃N₈O₈ 929.51; found: 929.4.

### Step 3: Synthesis of tert-butyl ((2²S,6³S,4S)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1′H-8-oxa-2(4,2)-morpholina-1(5,3)-indola-6(1,3)-pyridazinacycloundecaphane-4-yl)carbamate

To a solution of (S)-1-((S)-2-((tert-butoxycarbonyl)amino)-3-((S)-4-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)morpholin-2-yl)propanoyl)hexahydropyridazine-3-carboxylic acid (55.7 g, 59.95 mmol) and DIPEA (208.8 mL, 1.199 mol) in DCM (6500 mL) at 0° C. was added EDCI (229.9 g, 1.199 mol) and HOBt (40.5 g, 0.299 mol). The resulting mixture was stirred overnight at room temperature. The reaction mixture was quenched by the addition of cold H₂O (500 mL) and the aqueous layer was extracted with EtOAc (3×800 mL). The combined organic layers were washed with brine (2×500 mL), dried over Na₂SO₄, filtered, and concentrated under reduced pressure. The residue was purified by silica gel column chromatography to afford the desired product (35.0 g, 64% yield) as a solid. LCMS (ESI) m/z [M+H] calcd for C₄₇H₆₅F₃N₈O₇ 911.50; found: 911.3.

### Step 4: Synthesis of (2²S,6³S,4S)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

To a solution of tert-butyl ((2²S,6³S,4S)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate (33 g, 36.07 mmol) in DCM (180 mL) at 0° C. was added HCl in 1,4-dioxane (180 mL). The resulting mixture was stirred for 2 h at room temperature and then the mixture was concentrated under reduced pressure to afford the desired product (33 g, crude) as a solid. LCMS (ESI) m/z [M+H] calcd for C₄₂H₅₇F₃N₈O₅ 811.45; found: 811.3.

### Intermediate 30: Synthesis of (6³S,4S,Z)-4-amino-1²-(5-(4-cyclopropylpiperazin-1-yl)-2-((S)-1-methoxyethyl)pyridin-3-yl)-10,10-dimethyl-1-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

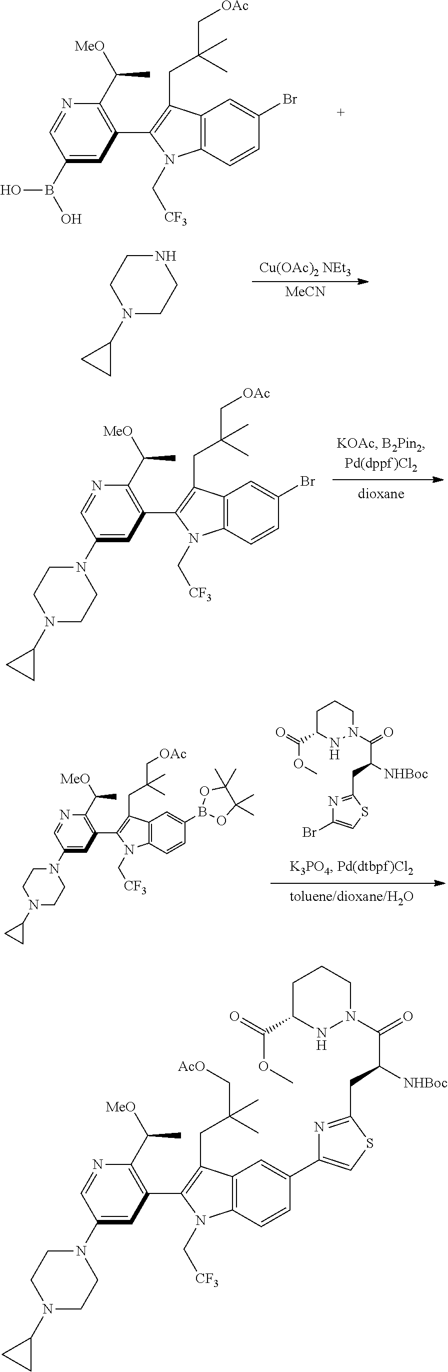

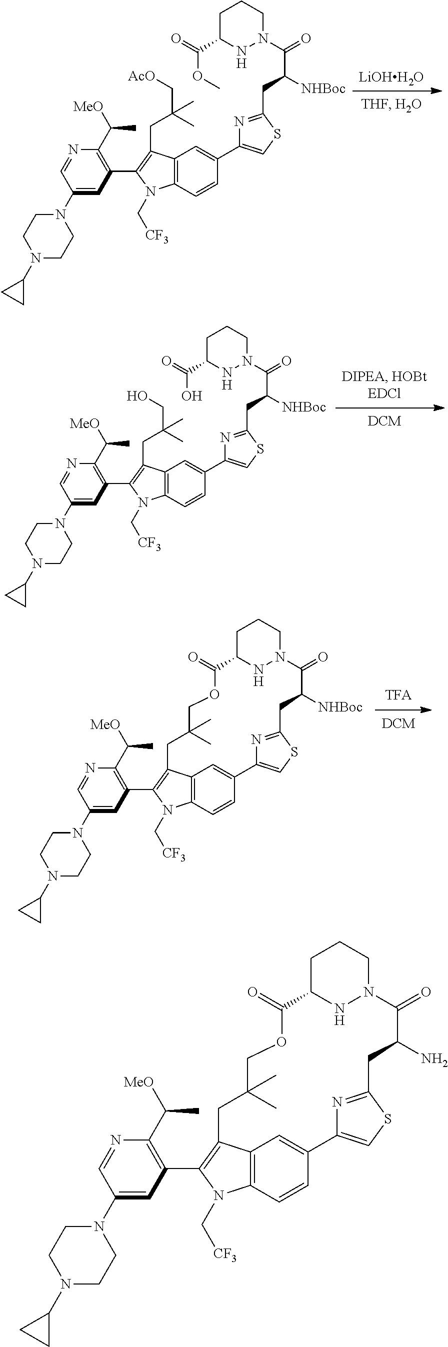

### Step 1: Synthesis of (S)-3-(5-bromo-2-(5-(4-cyclopropylpiperazin-1-yl)-2-(1-methoxyethyl)pyridin-3-yl)-1-(2,2,2-trifluoroethyl)-1H-indol-3-yl)-2,2-dimethylpropyl acetate

To a solution of (S)-(5-(3-(3-acetoxy-2,2-dimethylpropyl)-5-bromo-1-(2,2,2-trifluoroethyl)-1H-indol-2-yl)-6-(1-methoxyethyl)pyridin-3-yl)boronic acid (40 g, 61.514 mmol) and NEt₃ (12.45 g, 123.028 mmol) in MeCN (1000 mL) was added 4 Å MS (8 g) and 1-cyclopropylpiperazine (38.82 g, 307.570 mmol) in portions under an oxygen atmosphere. The resulting mixture was stirred for 1 h at room temperature under an oxygen atmosphere. To the above mixture was added Cu(OAc)₂ (22.35 g, 123.028 mmol) and then the vessel was evacuated, backfilled with oxygen, and then stirred overnight at room temperature. The resulting mixture was filtered and was concentrated under reduced pressure. The residue was diluted with EtOAc (300 mL) and the organic layer was washed with NH₃⋅6H₂O (4×100 mL), dried over Na₂SO₄, and concentrated under reduce pressure. The residue was purified by column chromatography (50% EtOAc/pet. ether) to afford the desired product (23.7 g, 56% yield) as a solid. LCMS (ESI) m/z [M+H] calcd for C₃₂H₄₀BrF₃N₄O₃ 665.23; found: 666.0.

### Step 2: Synthesis of (S)-3-(2-(5-(4-cyclopropylpiperazin-1-yl)-2-(1-methoxyethyl)pyridin-3-yl)-5-(4,4,5,5-tetramethyl-1,3,2-dioxaborolan-2-yl)-1-(2,2,2-trifluoroethyl)-1H-indol-3-yl)-2,2-dimethylpropyl acetate

To a solution of (S)-3-(5-bromo-2-(5-(4-cyclopropylpiperazin-1-yl)-2-(1-methoxyethyl)pyridin-3-yl)-1-(2,2,2-trifluoroethyl)-1H-indol-3-yl)-2,2-dimethylpropyl acetate (26 g, 39.063 mmol) and KOAc (13.42 g, 136.721 mmol) in dioxane (260 mL) was added B₂Pin₂ (37.7 g, 148.4 mmol) and Pd(dppf)Cl₂ (2.86 g, 3.906 mmol). The resulting mixture was evacuated and backfilled with argon then stirred at 90° C. for 3 h. The resulting mixture was filtered, the filter cake was washed with EtOAc (2×200 mL), and the filtrate was concentrated under reduced pressure. The residue was purified by silica gel column chromatography, (80% EtOAc/pet. ether) to afford the desired product (17 g, 61% yield) as a solid. LCMS (ESI) m/z [M+H] calcd for C₃₈H₅₂BF₃N₄O₅ 713.41; found: 713.3.

### Step 3: Synthesis of methyl (S)-1-((S)-3-(4-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)thiazol-2-yl)-2-((tert-butoxycarbonyl)amino)propanoyl)hexahydropyridazine-3-carboxylate

To a solution of (S)-3-(2-(5-(4-cyclopropylpiperazin-1-yl)-2-(1-methoxyethyl)pyridin-3-yl)-5-(4,4,5,5-tetramethyl-1,3,2-dioxaborolan-2-yl)-1-(2,2,2-trifluoroethyl)-1H-indol-3-yl)-2,2-dimethylpropyl acetate (17 g, 23.854 mmol), methyl (S)-1-((S)-3-(4-bromothiazol-2-yl)-2-((tert-butoxycarbonyl)amino)propanoyl)hexahydropyridazine-3-carboxylate (13.66 g, 28.625 mmol) and K₃PO₄ (12.66 g, 59.635 mmol) in toluene (170 mL), dioxane (57 mL) and H₂O (57 mL) was added 1,1′-bis(di-tert-butylphosphino) ferrocene palladium dichloride (1.55 g, 2.385 mmol) in portions under an argon atmosphere. To the mixture was added. The resulting mixture was stirred at 70° C. for 2 h. The mixture was filtered, the filter cake was washed with EtOAc (3×100 mL). The aqueous layer was extracted with EtOAc (3×200 mL), and the combined organic layers were washed with brine (2×200 mL), dried over Na₂SO₄, filtered, and concentrated under reduced pressure. The residue was purified by silica gel column chromatography (3% MeOH/DCM) to afford the desired product (20.4 g, 87% yield) as a solid. LCMS (ESI) m/z [M+H] calcd for C₄₉H₆₅F₃N₈O₈S 983.47; found: 983.6.

### Step 4: Synthesis of (S)-1-((S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)thiazol-2-yl)propanoyl)hexahydropyridazine-3-carboxylic acid

To a solution of methyl (S)-1-((S)-3-(4-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)thiazol-2-yl)-2-((tert-butoxycarbonyl)amino)propanoyl)hexahydropyridazine-3-carboxylate (20 g, 20.343 mmol) in THE (200 mL) was added a solution of LiOH (2.56 g, 61.029 mmol) in H₂O (61 mL) at 0° C. The resulting mixture was stirred overnight at room temperature. The mixture was then acidified to pH 6 with 1 N HCl (aq.) and was then extracted with EtOAc (3×100 mL). The combined organic layers were washed with brine (2×100 mL), dried over Na₂SO₄, filtered, and concentrated under reduced pressure to afford the crude product (18.8 g), which was used directly in the next step without purification. LCMS (ESI) m/z [M+H] calcd for C₄₆H₆₁F₃N₈O₇S 927.44; found: 927.3.

### Step 5: Synthesis of tert-butyl ((6³S,4S,Z)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-11H-8-oxa-2(4,2)-thiazola-1 (5,3)-indola-6(1,3)-pyridazinacycloundecaphane-4-yl)carbamate

To a solution of (S)-1-((S)-2-((tert-butoxycarbonyl)amino)-3-(4-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)thiazol-2-yl)propanoyl)hexahydropyridazine-3-carboxylic acid (15 g, 16.179 mmol), DIPEA (112.73 mL, 647.160 mmol), and HOBt (43.72 g, 323.580 mmol) in DCM (768 mL) at 0° C. was added EDCI (93.05 g, 485.370 mmol). The resulting mixture was stirred overnight at room temperature. The reaction mixture was quenched by the addition of cold H₂O (500 mL). The resulting mixture was extracted with EtOAc (3×500 mL), and the combined organic layers were washed with brine (2×500 mL), dried over Na₂SO₄, filtered, and concentrated under reduced pressure. The residue was purified by silica gel column chromatography (3% MeOH/DCM) to afford the desired product (7.5 g, 51% yield) as a solid. LCMS (ESI) m/z [M+H] calcd for C₄₆H₅₉F₃N₈O₆S 909.43; found: 909.3.

### Step 6: Synthesis of (6³S,4S,Z)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

To a solution of tert-butyl ((63S,4S,Z)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (8.2 g, 9.02 mmol) in DCM (80 mL) at 0° C. was added TFA (40 mL, 538.52 mmol). The resulting mixture was stirred for 2 h at room temperature. The mixture was then concentrated under reduced pressure and the residue was adjusted to pH 8 with sat. NaHCO₃ (aq.). The resulting mixture was extracted with EtOAc (3×300 mL), and the combined organic layers were washed with brine (300 mL), dried over Na₂SO₄, filtered, and concentrated under reduced pressure to afford the desired product (8.0 g, 98% yield) as a solid. LCMS (ESI) m/z [M+H] calcd for C₄₁H₅₁F₃NaO₄S 809.38; found: 809.5.

### Intermediate F31: Synthesis of (2²S,6³S,4S)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-11H-8-oxa-2(4,2)-morpholina-1(5,3)-indola-6(1,3)-pyridazinacycloundecaphane-5,7-dione

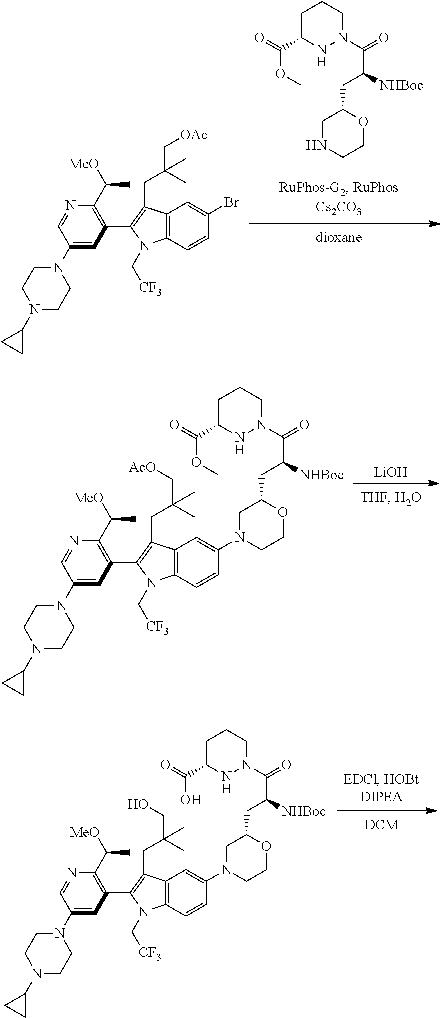

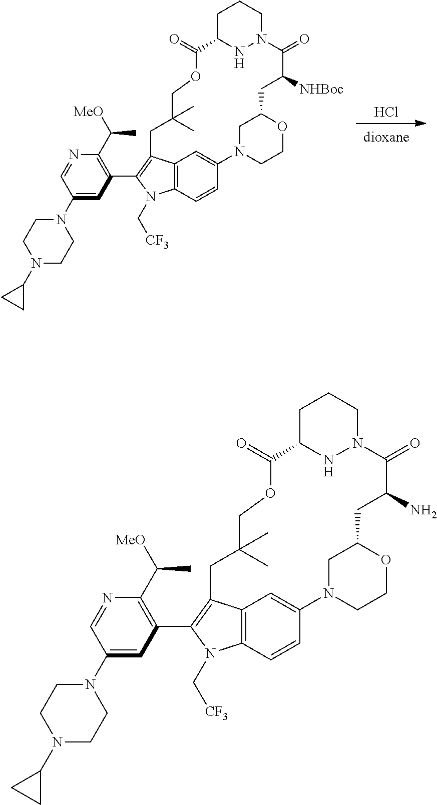

### Step 1: Synthesis of methyl (S)-1-((S)-3-((S)-4-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)morpholin-2-yl)-2-((tert-butoxycarbonyl)amino)propanoyl)hexahydropyridazine-3-carboxylate

To a solution of 3(S)-3-(5-bromo-2-(5-(4-cyclopropylpiperazin-1-yl)-2-(1-methoxyethyl)pyridin-3-yl)-1-(2,2,2-trifluoroethyl)-1H-indol-3-yl)-2,2-dimethylpropyl acetate (65.0 g, 97.66 mmol) and methyl (S)-1-((S)-2-((tert-butoxycarbonyl)amino)-3-((S)-morpholin-2-yl)propanoyl)hexahydropyridazine-3-carboxylate (78.2 g, 0.195 mol) in dioxane (650 mL) was added RuPhos (27.3 g, 58.60 mmol), RuPhos-G2-Pd (22.7 g, 29.30 mmol), and Cs₂CO₃ (95.5 g, 0.29 mol). The resulting mixture was stirred overnight at 80° C. The reaction mixture was then filtered, the filter cake was washed with EtOAc (3×300 mL), and the filtrate was concentrated under reduced pressure. The residue was purified by silica gel column chromatography to afford the desired product (63 g, 65% yield) as a solid. LCMS (ESI) m/z [M+H] calcd for C₅₀H₇₁F₃NaO₉ 985.54; found: 985.8.

### Step 2: Synthesis of (S)-1-((S)-2-((tert-butoxycarbonyl)amino)-3-((S)-4-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)morpholin-2-yl)propanoyl)hexahydropyridazine-3-carboxylic acid

To a solution of methyl (S)-1-((S)-3-((S)-4-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)morpholin-2-yl)-2-((tert-butoxycarbonyl)amino)propanoyl)hexahydropyridazine-3-carboxylate (79 g, 80.19 mmol) in THE (700 mL) was added a solution of LiOH⋅H₂O (16.7 g, 0.398 mol) in H₂O (150 mL) at 0° C. The resulting mixture was stirred for 5 h at room temperature. The mixture was then acidified to pH 5 with 1M HCl. The aqueous layer was extracted with DCM (3×500 mL) and the organic layer was dried over Na₂SO₄, filtered, and concentrated under reduced pressure to afford the desired product (70 g, crude) as a solid. LCMS (ESI) m/z [M+H] calcd for C₄₇H₆₇F₃N₈O₈ 929.51; found: 929.4.

### Step 3: Synthesis of tert-butyl ((2²S,6³S,4S)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate

To a solution of (S)-1-((S)-2-((tert-butoxycarbonyl)amino)-3-((S)-4-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)morpholin-2-yl)propanoyl)hexahydropyridazine-3-carboxylic acid (55.7 g, 59.95 mmol) and DIPEA (208.8 mL, 1.199 mol) in DCM (6500 mL) at 0° C. was added EDCI (229.9 g, 1.199 mol) and HOBt (40.5 g, 0.299 mol). The resulting mixture was stirred overnight at room temperature. The reaction mixture was quenched by the addition of cold H₂O (500 mL) and the aqueous layer was extracted with EtOAc (3×800 mL). The combined organic layers were washed with brine (2×500 mL), dried over Na₂SO₄, filtered, and concentrated under reduced pressure. The residue was purified by silica gel column chromatography to afford the desired product (35.0 g, 64% yield) as a solid. LCMS (ESI) m/z [M+H] calcd for C₄₇H₆₅F₃N₈O₇ 911.50; found: 911.3.

### Step 4: Synthesis of (2²S,6³S,4S)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

To a solution of tert-butyl ((2²S,6³S,4S)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate (33 g, 36.07 mmol) in DCM (180 mL) at 0° C. was added HCl in 1,4-dioxane (180 mL). The resulting mixture was stirred for 2 h at room temperature and then the mixture was concentrated under reduced pressure to afford the desired product (33 g, crude) as a solid. LCMS (ESI) m/z [M+H] calcd for C₄₂H₅₇F₃N₈O₅ 811.45; found: 811.3.

### Intermediate F32: Synthesis of (6³S,4S)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

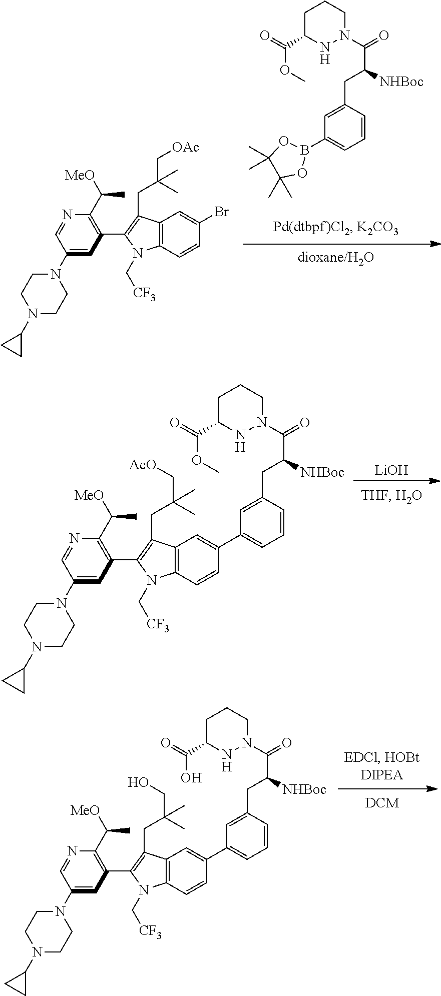

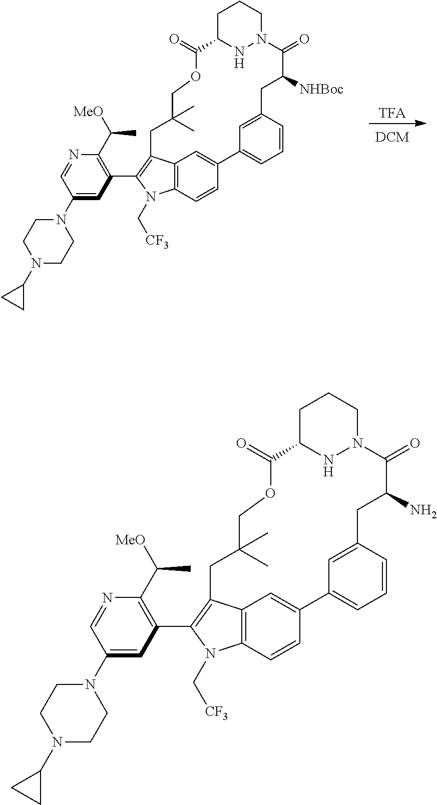

### Step 1: Synthesis of methyl (S)-1-((S)-3-(3-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)phenyl)-2-((tert-butoxycarbonyl)amino)propanoyl)hexahydropyridazine-3-carboxylate

To a solution of 3(S)-3-(5-bromo-2-(5-(4-cyclopropylpiperazin-1-yl)-2-(1-methoxyethyl)pyridin-3-yl)-1-(2,2,2-trifluoroethyl)-1H-indol-3-yl)-2,2-dimethylpropyl acetate (1 g, 1.502 mmol) methyl (S)-1-((S)-2-((tert-butoxycarbonyl)amino)-3-(3-(4,4,5,5-tetramethyl-1,3,2-dioxaborolan-2-yl)phenyl)propanoyl)hexahydropyridazine-3-carboxylate (1.14 g, 2.253 mmol) in 1,4-dioxane (10 mL) and H₂O (2 mL) was added K₂CO₃ (415.28 mg, 3.004 mmol) and Pd(dtbpf)Cl₂ (97.92 mg, 0.150 mmol). The resulting mixture was stirred for 3 h at 65° C. The precipitated solids were collected by filtration and washed with DCM (30 mL). The resulting mixture was extracted with EtOAc (3×50 mL). The combined organic layers were washed with brine (2×20 mL), dried over Na₂SO₄, filtered, and concentrated under reduced pressure. The residue was purified by silica gel column chromatography (10% MeOH/DCM) to afford the desired product (860 mg, 90% yield) as a solid. LCMS (ESI) m/z [M+H] calcd for C₅₂H₆₈F₃N₇O₈ 976.52; found: 976.9.

### Step 2: Synthesis of (S)-1-((S)-2-((tert-butoxycarbonyl)amino)-3-(3-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)phenyl)propanoyl)hexahydropyridazine-3-carboxylic acid

To a solution of methyl (S)-1-((S)-3-(3-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)phenyl)-2-((tert-butoxycarbonyl)amino)propanoyl)hexahydropyridazine-3-carboxylate (6.8 g, 6.963 mmol) in THE (68 mL) was added a solution of LiOH⋅H₂O (4.096 mmol) in H₂O (13.9 mL) at 0° C. The resulting mixture was stirred overnight and was then acidified to pH 5 with 1M HCl. The resulting mixture was extracted with DCM (3×50 mL) and the combined organic layers were washed with brine (2×50 mL), dried over Na₂SO₄, filtered, and concentrated under reduced pressure to afford the desired product (6 g, 90% yield) as a solid. LCMS (ESI) m/z [M+H] calcd for C₄₉H₆₄F₃N₇O₇ 920.49; found: 920.9.

### Step 3: Synthesis of tert-butyl ((63S,4S)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of (S)-1-((S)-2-((tert-butoxycarbonyl)amino)-3-(3-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)phenyl)propanoyl)hexahydropyridazine-3-carboxylic acid (5 g, 5.434 mmol) in DCM (40 mL) at 0° C. was added DIPEA (28.09 g, 217.360 mmol) and HOBt (7.34 g, 54.34 mmol). To the mixture was added a solution of EDCI (31.25 g, 163.020 mmol) in DCM (10 mL). The resulting mixture was warmed to room temperature and stirred overnight. The mixture was concentrated under reduced pressure and the residue was taken up in EtOAc (100 mL). The organic layer was washed with brine (2×100 mL), dried over Na₂SO₄, filtered, and concentrated under reduced pressure. The residue was purified by silica gel column chromatography (3% MeOH/DCM) to afford the desired product (4.2 g, 79% yield) as a solid.

LCMS (ESI) m/z [M+H] calcd for C₄₉H₆₂F₃N₇O₆ 902.48; found: 902.1.

### Step 4: Synthesis of (6³S,4S)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a solution of tert-butyl ((63S,4S)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.8 g, 1.995 mmol) in DCM (16 mL) at 0° C. was added TFA (4 mL). The resulting mixture was stirred at 0° C. for 1 h and then the mixture was neutralized to pH 7 with sat. NaHCO₃ (aq). The resulting mixture was extracted with DCM (100 mL) and the combined organic layers were washed with brine (2×100 mL), dried over Na₂SO₄, filtered, and concentrated under reduced pressure to afford the desired product (1.5 g, 89% yield) as a solid. LCMS (ESI) m/z [M+H] calcd for C₄₄H₅₄F₃N₇O₄ 802.43; found: 802.8. Intermediate F33: Synthesis of (6³S,4S,Z)-4-amino-1²-(5-(4-cyclopropylpiperazin-1-yl)-2-((S)-1-methoxyethyl)pyridin-3-yl)-1¹-ethyl-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

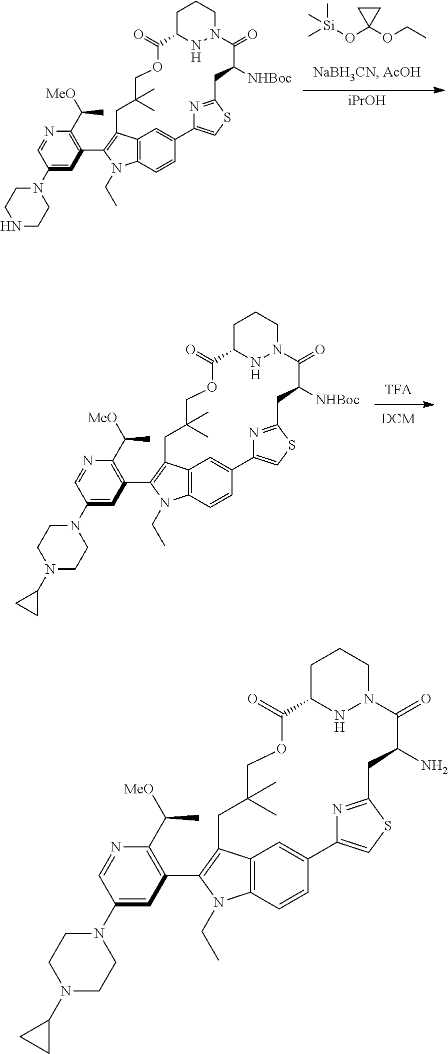

### Step 1: Synthesis of tert-butyl ((6³S,4S,Z)-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)-pyridazinacycloundecaphane-4-yl)carbamate

To a solution of tert-butyl ((6³S,4S,Z)-1¹-ethyl-1²-(2-((S)-1-methoxyethyl)-5-(piperazin-1-yl)pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)- pyridazinacycloundecaphane-4-yl)carbamate (2 g, 2.454 mmol) and (1-ethoxycyclopropoxy)trimethylsilane (0.86 g, 4.908 mmol) in 2-propanol (20 mL) at room temperature was added NaBH₃CN (0.46 g, 7.362 mmol) and AcOH (0.28 mL, 4.908 mmol). The resulting mixture was stirred at 50° C. for 16 h and then reaction mixture was cooled to 0° C. and sat. NH₄Cl (30 mL) was added. The resulting mixture was extracted with EtOAc (2×20 mL) and the combined organic layers were washed with brine (2×20 mL), dried over Na₂SO₄, filtered, and concentrated under reduced pressure. The residue was purified by silica gel column chromatography (3% MeOH/DCM) to afford the desired product (1.5 g, 71% yield) as a solid. LCMS (ESI) m/z [M+H] calcd for C₄₆H₆₂N₈O₆S 855.46; found: 856.4.

### Step 2: Synthesis of (6³S,4S,Z)-4-amino-1²-(5-(4-cyclopropylpiperazin-1-yl)-2-((S)-1-methoxyethyl)pyridin-3-yl)-1¹-ethyl-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

A solution of tert-butyl ((6³S,4S,Z)-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (1.56 g, 1.824 mmol) and TFA (4 mL) in DCM (16 mL) was stirred at room temperature for 2 h and was then concentrated under reduced pressure. The residue was dissolved in EtOAc (30 mL) and the mixture was basified to pH 8 with sat. NaHCO₃ (aq). The aqueous layer was extracted with EtOAc (3×40 mL) and the combined organic layers were washed with brine (2×20 mL), dried over Na₂SO₄, filtered, and concentrated under reduced pressure to afford the desired product (1.36 g, crude) as a solid. LCMS (ESI) m/z [M+H] calcd for C₄₁H₅₄NaO₄S 755.41; found: 755.3.

### Intermediate F34: Synthesis of (2²S,6³S,4S)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

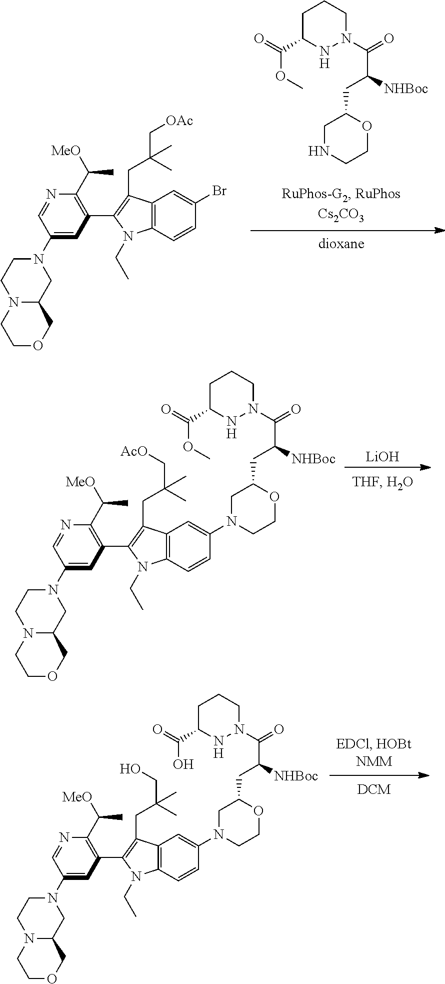

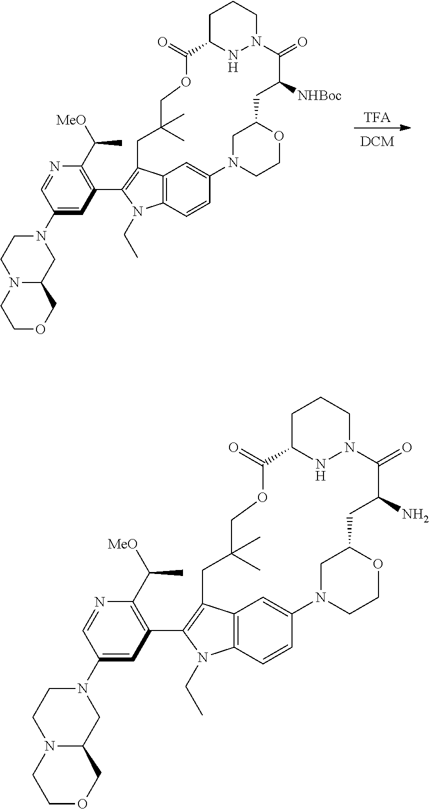

### Step 1: Synthesis of methyl (S)-1-((S)-3-((S)-4-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)morpholin-2-yl)-2-((tert-butoxycarbonyl)amino)propanoyl)hexahydropyridazine-3-carboxylate

To a solution of 3-(5-bromo-1-ethyl-2-(5-((S)-hexahydropyrazino[2,1-c][1,4]oxazin-8(1H)-yl)-2-((S)-1-methoxyethyl)pyridin-3-yl)-1H-indol-3-yl)-2,2-dimethylpropyl acetate (15 g, 23.900 mmol) and methyl (S)-1-((S)-2-((tert-butoxycarbonyl)amino)-3-((S)-morpholin-2-yl)propanoyl)hexahydropyridazine-3-carboxylate (12.44 g, 31.070 mmol) in dioxane (150 mL) was added RuPhos (2.23 g, 4.780 mmol), RuPhos-G2-Pd (1.86 g, 2.390 mmol), and Cs₂CO₃ (3.64 g, 47.800 mmol). The resulting mixture was stirred overnight at 90° C. The reaction mixture was then filtered, the filter cake was washed with EtOAc (3×100 mL), and the filtrate was concentrated under reduced pressure. The residue was purified by silica gel column chromatography (9% MeOH/DCM) to afford the desired product (15.3 g, 33% yield) as a solid. LCMS (ESI) m/z [M+H] calcd for C₅₀H₇₄N₈O₁₀ 947.56; found: 947.4.

### Step 2: Synthesis of (S)-1-((S)-2-((tert-butoxycarbonyl)amino)-3-((S)-4-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)morpholin-2-yl)propanoyl)hexahydropyridazine-3-carboxylic acid

To a solution of methyl (S)-1-((S)-3-((S)-4-(3-(3-acetoxy-2,2-dimethylpropyl)-1-ethyl-2-(5-((S)-hexahydropyrazino[2,1-c][1,4]oxazin-8(1H)-yl)-2-((S)-1-methoxyethyl)pyridin-3-yl)-1H-indol-5-yl)morpholin-2-yl)-2-((tert-butoxycarbonyl)amino)propanoyl)hexahydropyridazine-3-carboxylate (7.6 g, 8.024 mmol) in THE (34 mL) and H₂O (34) was added LiOH⋅H₂O (0.96 g, 40.120 mmol). The resulting mixture was stirred overnight at room temperature. The mixture was then acidified to pH 5 with HCl (1M). The aqueous layer was extracted with DCM (3×100 mL) and the organic layer was washed with brine (3×100 mL), dried over Na₂SO₄, filtered, and concentrated under reduced pressure to afford the desired product (6.4 g, 89% yield) as a solid. LCMS (ESI) m/z [M+H] calcd for C₄₇H₇₀N₈O₉ 891.54; found: 891.5.

### Step 3: Synthesis of tert-butyl ((2²S,6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate

To a solution of (S)-1-((S)-2-((tert-butoxycarbonyl)amino)-3-((S)-4-(1-ethyl-2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1H-indol-5-yl)morpholin-2-yl)propanoyl)hexahydropyridazine-3-carboxylic acid (6.4 g, 7.182 mmol) and NMM (58.11 g, 574.560 mmol) in DCM (640 mL) at 0° C. was added EDCI (82.61 g, 430.920 mmol) and HOBt (14.6 g, 75.9 mmol). The resulting mixture was stirred overnight at room temperature. The reaction mixture was concentrated under reduced pressure and taken up in EtOAc (3×300 mL). The organic layer was washed with brine (3×200 mL), dried over Na₂SO₄, filtered, and concentrated under reduced pressure. The residue was purified by silica gel column chromatography (8% MeOH/DCM) to afford the desired product (3.2 g, 51% yield) as a solid. LCMS (ESI) m/z [M+H] calcd for C₄₇H₆₈N₈O₈ 873.53; found: 873.4.

### Step 4: Synthesis of (2²S,6³S,4S)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

To a solution tert-butyl ((2²S,6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate (1 g, 1.145 mmol) in DCM (20 mL) at 0° C. was added TFA (10 mL). The resulting mixture was stirred at 0° C. for 3 h and was then concentrated under reduced to afford the desired product as a solid. LCMS (ESI) m/z [M+H] calcd for C₄₂H₆₀N₈O₆ 773.47; found: 773.5.

### Intermediate F35: Synthesis of (6³S,4S)-4-amino-1²-(5-(4-cyclopropylpiperazin-1-yl)-2-((S)-1-methoxyethyl)pyridin-3-yl)-1¹-ethyl-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

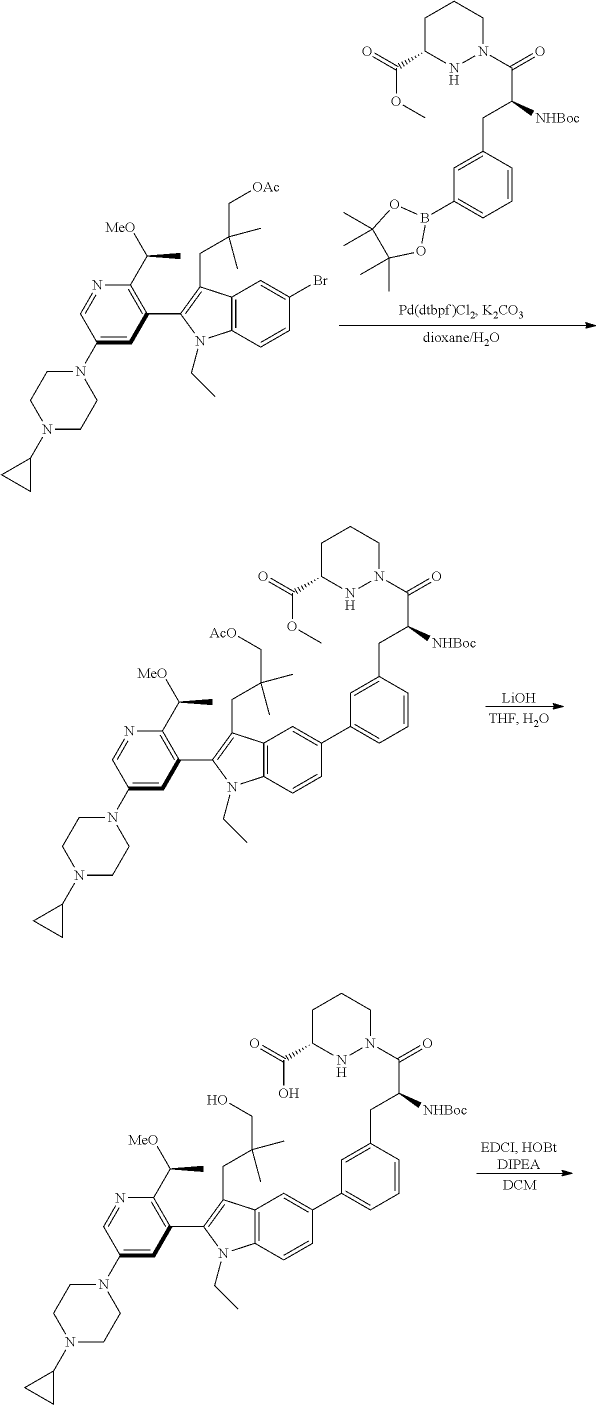

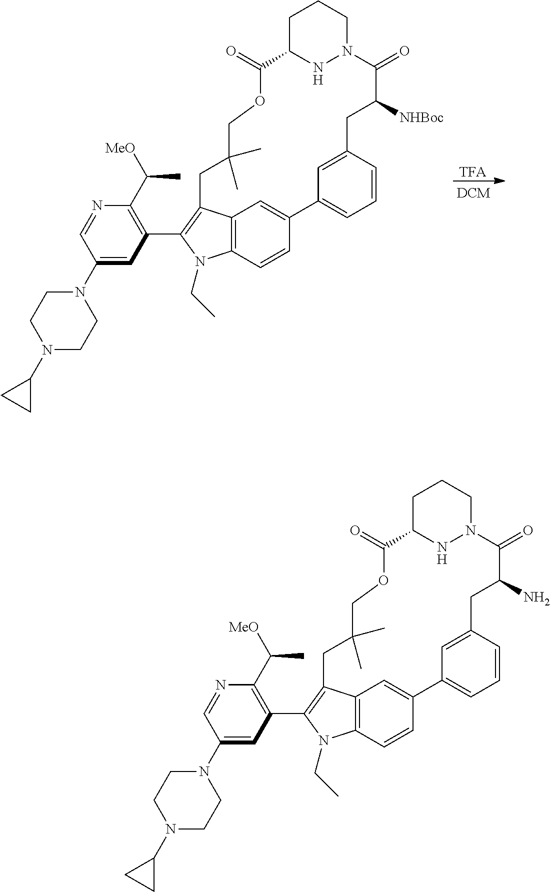

### Step 1: Synthesis of methyl (S)-1-((S)-3-(3-(3-(3-acetoxy-2,2-dimethylpropyl)-2-(5-(4-cyclopropylpiperazin-1-yl)-2-((S)-1-methoxyethyl)pyridin-3-yl)-1-ethyl-1H-indol-5-yl)phenyl)-2-((tert-3, C₃ butoxycarbonyl)amino)propanoyl)hexahydropyridazine-3-carboxylate

To a solution of (S)-3-(5-bromo-2-(5-(4-cyclopropylpiperazin-1-yl)-2-(1-methoxyethyl)pyridin-3-yl)-1-ethyl-1H-indol-3-yl)-2,2-dimethylpropyl acetate (10 g, 16.350 mmol) and methyl (S)-1-((S)-2-((tert-butoxycarbonyl)amino)-3-(3-(4,4,5,5-tetramethyl-1,3,2-dioxaborolan-2-yl)phenyl)propanoyl)hexahydropyridazine-3-carboxylate (12.69 g, 24.525 mmol) in 1,4-dioxane (110 mL) and H₂O (20 mL) was added K₂CO₃ (4.52 g, 32.700 mmol) and Pd(dtbpf)Cl₂ (1.07 g, 1.635 mmol). The resulting mixture was stirred for 3 h at 70° C. The precipitated solids were collected by filtration and washed with DCM (2×200 mL). The resulting mixture was extracted with EtOAc (100 mL). The combined organic layers were washed with brine (2×100 mL), dried over Na₂SO₄, filtered, and concentrated under reduced pressure. The residue was purified by silica gel column chromatography (10% MeOH/DCM) to afford the desired product (8.5 g, 56% yield) as a solid. LCMS (ESI) m/z [M+H] calcd for C₅₂H₇₁N₇O₈ 922.55; found: 922.7.

### Step 2: Synthesis of (S)-1-((S)-2-((tert-butoxycarbonyl)amino)-3-(3-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)phenyl)propanoyl)hexahydropyridazine-3-carboxylic acid

To a solution of methyl (S)-1-((S)-3-(3-(3-(3-acetoxy-2,2-dimethylpropyl)-2-(5-(4-cyclopropylpiperazin-1-yl)-2-((S)-1-methoxyethyl)pyridin-3-yl)-1-ethyl-1H-indol-5-yl)phenyl)-2-((tert-butoxycarbonyl)amino)propanoyl)hexahydropyridazine-3-carboxylate (7 g, 7.591 mmol) in THE (70 mL) was added a solution of LiOH⋅H₂O (0.96 g, 22.773) in H₂O (22 mL) at 0° C. The resulting mixture was stirred overnight and was then acidified to pH 6 with 1M HCl. The resulting mixture was extracted with DCM (2×100 mL) and the combined organic layers were washed with brine (2×100 mL), dried over Na₂SO₄, filtered, and concentrated under reduced pressure to afford the desired product as a solid.

LCMS (ESI) m/z [M+H] calcd for C₄₉H₆₇N₇O₇ 866.52; found: 866.4.

### Step 3: Synthesis of tert-butyl ((6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

To a solution of (S)-1-((S)-2-((tert-butoxycarbonyl)amino)-3-(3-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)phenyl)propanoyl)hexahydropyridazine-3-carboxylic acid (6 g, 6.927 mmol) in DCM (240 mL) at 0° C. was added DIPEA (35.81 g, 277.080 mmol) and HOBt (9.36 g, 69.270 mmol). To the mixture was added a solution of EDCI (39.84 g, 207.810 mmol) in DCM (240 mL). The resulting mixture was warmed to room temperature and stirred overnight. The mixture was concentrated under reduced pressure and the residue was taken up in EtOAc (200 mL). The organic layer was washed with brine, dried over Na₂SO₄, filtered, and concentrated under reduced pressure. The residue was purified by prep-TLC (8% MeOH/DCM) to afford the desired product (43.8 g, 64% yield) as a solid. LCMS (ESI) m/z [M+H] calcd for C₄₉H₆₅N₇O₆ 848.51; found: 848.7.

### Step 4: Synthesis of (6³S,4S)-4-amino-1²-(5-(4-cyclopropylpiperazin-1-yl)-2-((S)-1-methoxyethyl)pyridin-3-yl)-1¹-ethyl-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a solution of tert-butyl ((63S,4S)-1²-(5-(4-cyclopropylpiperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)carbamate (1.5 g, 1.769 mmol) in DCM (15 mL) at 0° C. was added TFA (7 mL). The resulting mixture was stirred at 0° C. for 1 h and then the mixture was neutralized to pH 8 with sat. NaHCO₃ (aq). The resulting mixture was extracted with DCM (3×100 mL) and the combined organic layers were washed with brine (3×100 mL), dried over Na₂SO₄, filtered, and concentrated under reduced pressure to afford the desired product as a solid. LCMS (ESI) m/z [M+H] calcd for C₄₄H₅₇N₇O₄ 748.45; found: 748.4.

### Intermediate F36: Synthesis of (6³S,4S,Z)-4-amino-1²-(5-((S)-hexahydropyrazino[2,1-c][1,4]oxazin-8(1H)-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

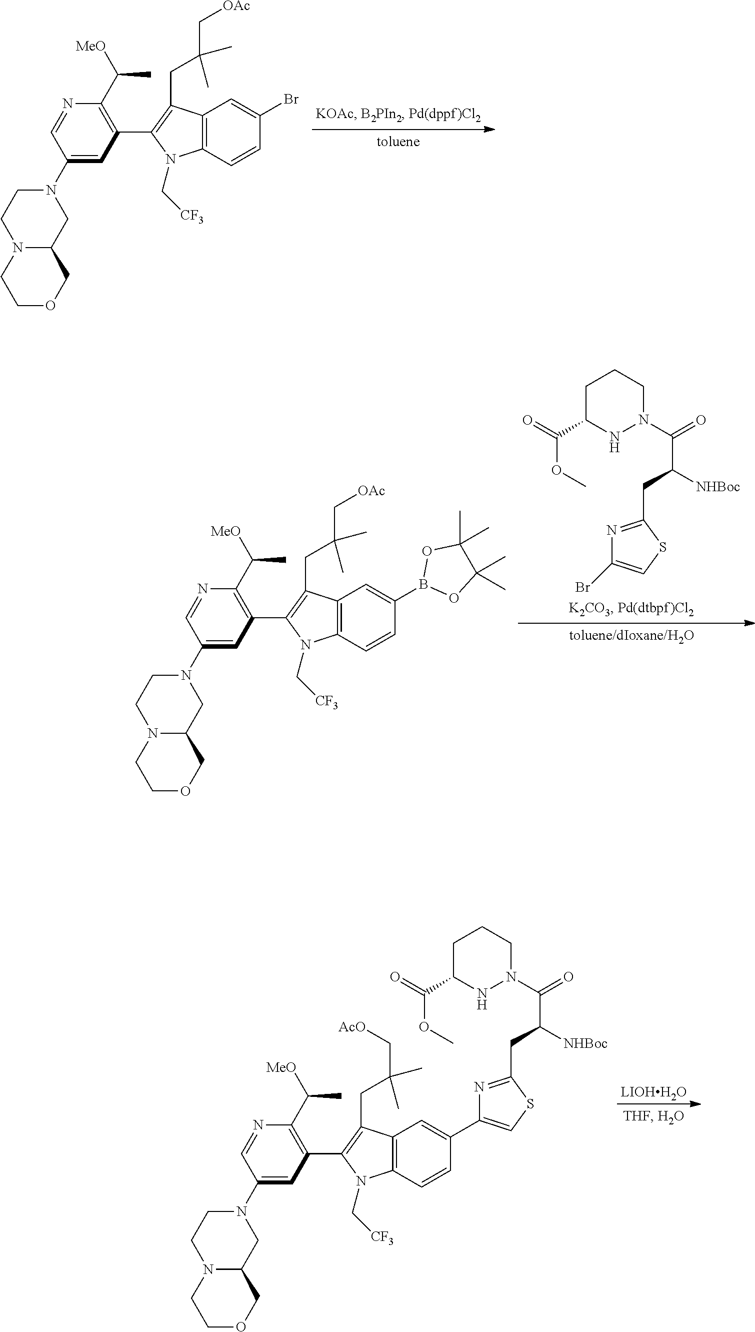

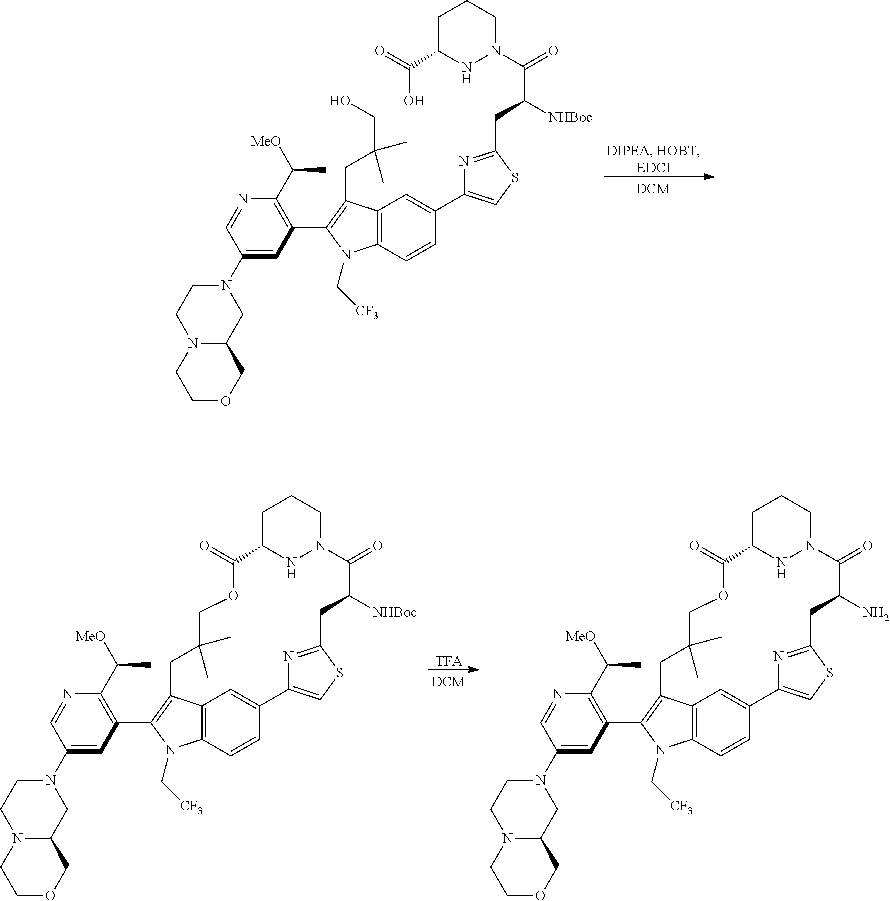

### Step 1: Synthesis of 3-(2-(5-((S)-hexahydropyrazino[2,1-c][1,4]oxazin-8(1H)-yl)-2-((S)-1-methoxyethyl)pyridin-3-yl)-5-(4,4,5,5-tetramethyl-1,3,2-dioxaborolan-2-yl)-1-(2,2,2-trifluoroethyl)-1H-indol-3-yl)-2,2-dimethylpropyl acetate

To a solution of 3-(5-bromo-2-(5-((S)-hexahydropyrazino[2,1-c][1,4]oxazin-8(1H)-yl)-2-((S)-1-methoxyethyl)pyridin-3-yl)-1-(2,2,2-trifluoroethyl)-1H-indol-3-yl)-2,2-dimethylpropyl acetate (26.5 g, 38.879 mmol), KOAc (9.54 g, 97.197 mmol) and 4,4,5,5-tetramethyl-2-(tetramethyl-1,3,2-dioxaborolan-2-yl)-1,3,2-dioxaborolane (19.75 g, 77.758 mmol) in toluene (300 mL) was added Pd(dppf)Cl₂ (2.84 g, 3.888 mmol) in portions at room temperature under an argon atmosphere. The resulting mixture was stirred for 3 h at 90° C. The resulting mixture was filtered, the filter cake was washed with DCM (3×500 mL). The filtrate was concentrated under reduced pressure. The residue was purified by silica gel column chromatography (20% EtOAc/pet. ether) to afford the product (325 g, 83% yield) as a solid. LCMS (ESI) m/z [M+H] calcd for C₃₈H₅₂BF₃N₄O₆: 729.40; found: 729.5.

### Step 2: Synthesis of methyl (S)-1-((S)-3-(4-(3-(3-acetoxy-2,2-dimethylpropyl)-2-(5-((S)-hexahydropyrazino[2,1-c][1,4]oxazin-8(1H)-yl)-2-((S)-1-methoxyethyl)pyridin-3-yl)-1-(2,2,2-trifluoroethyl)-1H-indol-5-yl)thiazol-2-yl)-2-((tert-butoxycarbonyl)amino)propanoyl)hexahydropyridazine-3-carboxylate

To a stirred mixture of 3-(2-(5-((S)-hexahydropyrazino[2,1-c][1,4]oxazin-8(1H)-yl)-2-((S)-1-methoxyethyl)pyridin-3-yl)-5-(4,4,5,5-tetramethyl-1,3,2-dioxaborolan-2-yl)-1-(2,2,2-trifluoroethyl)-1H-indol-3-yl)-2,2-dimethylpropyl acetate (25 g, 34.310 mmol), methyl (S)-1-((S)-3-(4-bromothiazol-2-yl)-2-((tert-butoxycarbonyl)amino)propanoyl)hexahydropyridazine-3-carboxylate (19.65 g, 41.172 mmol), and K₂CO₃ (11.85 g, 85.775 mmol) in toluene (200 mL) were added dioxane (100 mL) and H₂O (50 mL) was added Pd(dtbpf)Cl₂ (2.24 g, 3.431 mmol). The resulting mixture was stirred for 2 h at 70° C. The resulting mixture was filtered, the filter cake was washed with DCM (3×500 mL). The filtrate was concentrated under reduced pressure. The resulting mixture was extracted with DCM (3×200 mL). The combined organic layers were washed with brine, dried over anhydrous Na₂SO₄. After filtration, the filtrate was concentrated under reduced pressure. The residue was purified by silica gel column chromatography (20% EtOAc/pet. ether) to afford the desired product (32 g, 84% yield) as a solid. LCMS (ESI) m/z [M+H] calcd for C₄₉H₆₅F₃N₈O₉S: 999.46; found: 999.8.

### Step 3: Synthesis of (S)-1-((S)-2-((tert-butoxycarbonyl)amino)-3-(4-(2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1-(2,2,2-trifluoroethyl)-1H-indol-5-yl)thiazol-2-yl)propanoyl)hexahydropyridazine-3-carboxylic acid

To a solution of methyl (S)-1-((S)-3-(4-(3-(3-acetoxy-2,2-dimethylpropyl)-2-(5-((S)-hexahydropyrazino[2,1-c][1,4]oxazin-8(1H)-yl)-2-((S)-1-methoxyethyl)pyridin-3-yl)-1-(2,2,2-trifluoroethyl)-1H-indol-5-yl)thiazol-2-yl)-2-((tert-butoxycarbonyl)amino)propanoyl)hexahydropyridazine-3-carboxylate (32 g, 32.027 mmol) in THE (320 mL) and H₂O (300 mL) at 0° C. was added LiOH⋅H₂O (5.38 g, 128.108 mmol). The resulting mixture was stirred overnight at room temperature. The mixture was neutralized to pH 7 with HCl (aq.). The resulting mixture was extracted with DCM (3×500 mL). The combined organic layers were washed with brine (3×500 mL), dried over anhydrous Na₂SO₄. After filtration, the filtrate was concentrated under reduced pressure to afford the product (30 g, 89% yield) as a solid. LCMS (ESI) m/z [M+H] calcd for C₄₆H₆₁F₃N₈O₈S: 943.43; found: 943.8

### Step 4: Synthesis of tert-butyl ((6³S,4S,Z)-12-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

To a solution of (S)-1-((S)-2-((tert-butoxycarbonyl)amino)-3-(4-(2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1-(2,2,2-trifluoroethyl)-1H-indol-5-yl)thiazol-2-yl)propanoyl)hexahydropyridazine-3-carboxylic acid (30 g, 31.810 mmol), HOBt (25.79 g, 190.860 mmol) and DIPEA (164.45 g, 1272.400 mmol) in DCM (3 L) at 0° C. was added EDCI (182.94 g, 954.300 mmol) under an argon atmosphere. The resulting mixture was stirred for overnight at room temperature and then cold H₂O (5 L) was added. Then the mixture was extracted with DCM (3×1 L) and the combined organic layers were washed with brine (3×1 L), dried over Na₂SO₄, filtered, and concentrated under reduced pressure. The residue was purified by silica gel column chromatography (20% EtOAc/pet. ether) to afford the desired product (20 g, 64% yield) as a solid. LCMS (ESI) m/z [M+H] calcd for C₄₆H₅₉F₃N₈O₇S: 925.43; found: 925.5

### Step 5: Synthesis of (6³S,4S,Z)-4-amino-1²-(5-((S)-hexahydropyrazino[2,1-c][1,4]oxazin-8(1H)-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

To a solution of tert-butyl ((6³S,4S,Z)-12-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (977 mg, 0.843 mmol) in DCM (8 mL,) was added TFA (8 mL) dropwise at 0° C. under an argon atmosphere. The resulting mixture was stirred at 0° C. for 1 h. The mixture was basified to pH 8 with sat. aq. NaHCO₃. The resulting mixture was extracted with DCM (3×50 mL). The combined organic layers were washed with brine (3×50 mL), dried over Na₂SO₄, filtered, and concentrated under reduced pressure to afford the desired product (766 mg, 88% yield) as a solid. LCMS (ESI) m/z [M+H] calcd for C₄₁H₅₁F₃NaO₅S: 825.38; found: 825.6.

### Intermediate F37: Synthesis of (2²S,6³S,4S)-4-amino-1²-(5-((S)-hexahydropyrazino[2,1-c][1,4]oxazin-8(1H)-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

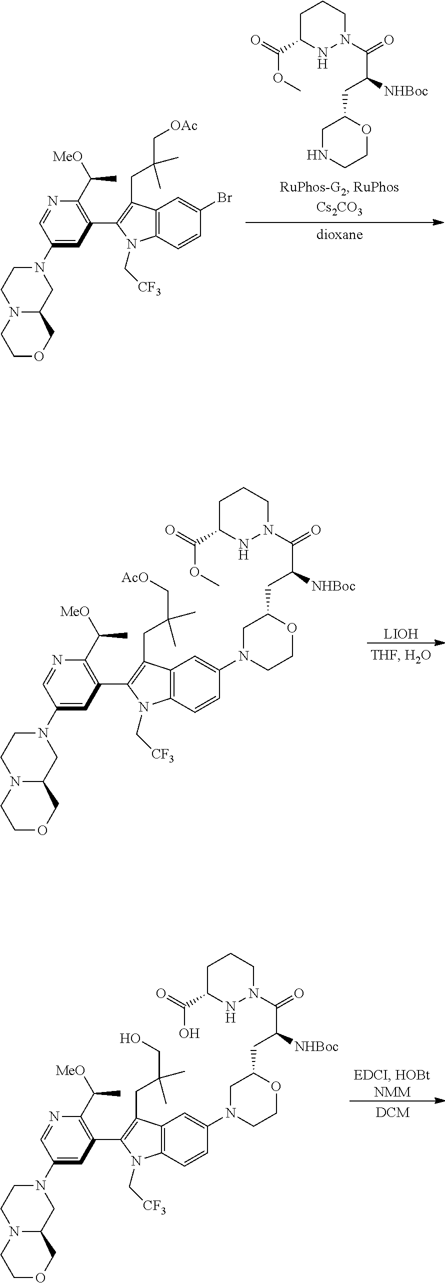

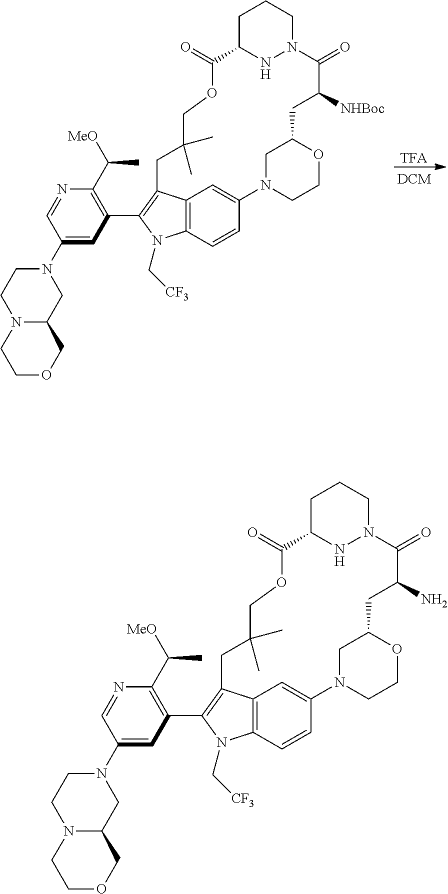

### Step 1: Synthesis of methyl (S)-1-((S)-3-((S)-4-(3-(3-acetoxy-2,2-dimethylpropyl)-2-(5-((S)-hexahydropyrazino[2,1-c][1,4]oxazin-8(1H)-yl)-2-((S)-1-methoxyethyl)pyridin-3-yl)-1-(2,2,2-trifluoroethyl)-1H-indol-5-yl)morpholin-2-yl)-2-((tert-butoxycarbonyl)amino)propanoyl)hexahydropyridazine-3-carboxylate

To a solution of 3-(5-bromo-2-(5-((S)-hexahydropyrazino[2,1-c][1,4]oxazin-8(1H)-yl)-2-((S)-1-methoxyethyl)pyridin-3-yl)-1-(2,2,2-trifluoroethyl)-1H-indol-3-yl)-2,2-dimethylpropyl acetate (9.5 g, 13.938 mmol) and methyl (S)-1-((S)-2-((tert-butoxycarbonyl)amino)-3-((S)-morpholin-2-yl)propanoyl)hexahydropyridazine-3-carboxylate (11.16 g, 27.876 mmol) in dioxane (95 mL) was added RuPhos (1.30 g, 2.788 mmol), RuPhos-G2-Pd (1.08 g, 1.394 mmol), and Cs₂CO₃ (9.08 g, 27.876 mmol). The resulting mixture was at 80° C. for 3 h. The reaction mixture was then filtered, the filter cake was washed with EtOAc (3×300 mL), and the filtrate was washed with brine (3×100 mL), dried over Na₂SO₄, filtered, and concentrated under reduced pressure. The residue was purified by silica gel column chromatography (5% MeOH/DCM) to afford the desired product (10 g, 70% yield) as a solid. LCMS (ESI) m/z [M+H] calcd for C₅₀H₇₁F₃N₈O₁₀ 1001.53; found: 1001.7.

### Step 2: Synthesis of (S)-1-((S)-2-((tert-butoxycarbonyl)amino)-3-((S)-4-(2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1-(2,2,2-trifluoroethyl)-1H-indol-5-yl)morpholin-2-yl)propanoyl)hexahydropyridazine-3-carboxylic acid

To a solution of methyl (S)-1-((S)-3-((S)-4-(3-(3-acetoxy-2,2-dimethylpropyl)-2-(5-((S)-hexahydropyrazino[2,1-c][1,4]oxazin-8(1H)-yl)-2-((S)-1-methoxyethyl)pyridin-3-yl)-1-(2,2,2-trifluoroethyl)-1H-indol-5-yl)morpholin-2-yl)-2-((tert-butoxycarbonyl)amino)propanoyl)hexahydropyridazine-3-carboxylate (10 g, 9.988 mmol) in THE (50 mL) and H₂O (50 mL) at 0° C. was added LiOH⋅H₂O (2.10 g, 49.940 mmol). The resulting mixture was stirred overnight at room temperature and then H₂O (100 mL) was added. The aqueous layer was extracted with MTBE (3×300 mL) and then the aqueous layer was acidified to pH 6 with HCl (1M) and extracted with DCM (3×500 mL). The combined organic layers were washed with brine (3×200 mL), dried over Na₂SO₄, filtered, and concentrated under reduced pressure to afford the desired product (7.1 g) as a solid. LCMS (ESI) m/z [M+H] calcd for C₄₇H₆₇F₃N₈O₉ 945.51; found: 945.3.

### Step 3: Synthesis of tert-butyl ((2²S,6³S,4S)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate

To a solution of (S)-1-((S)-2-((tert-butoxycarbonyl)amino)-3-((S)-4-(2-(5-((S)-hexahydropyrazino[2,1-c][1,4]oxazin-8(1H)-yl)-2-((S)-1-methoxyethyl)pyridin-3-yl)-3-(3-hydroxy-2,2-dimethylpropyl)-1-(2,2,2-trifluoroethyl)-1H-indol-5-yl)morpholin-2-yl)propanoyl)hexahydropyridazine-3-carboxylic acid (7.1 g, 7.512 mmol) and NMM (12.16 g, 120.192 mmol) in DCM (710 mL) at 0° C. was added EDCI (11.52 g, 60.096 mmol) and HOBt (4.06 g, 30.048 mmol). The resulting mixture was stirred at room temperature overnight and then H₂O (500 mL) was added. The resulting mixture was extracted with DCM (3×500 mL) and the combined organic layers were washed with brine (3×1 L), dried over Na₂SO₄, filtered, and concentrated under reduced pressure. The residue was purified by silica gel column chromatography (8% MeOH/DCM) to afford the desired product (3 g, 48% yield) as a solid. LCMS (ESI) m/z [M+H] calcd for C₄₇H₆₅F₃N₈O₈ 927.50; found: 927.3.

### Step 4: Synthesis of (2²S,6³S,4S)-4-amino-1²-(5-((S)-hexahydropyrazino[2,1-c][1,4]oxazin-8(1H)-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione

To a solution tert-butyl ((2²S,6³S,4S)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)carbamate (3 g, 3.236 mmol) in DCM (30 mL) at 0° C. was added TFA (15 mL). The resulting mixture was stirred at 0° C. for 2 h and was then basified to pH 8 with sat. NaHCO₃ (aq.). The resulting mixture was extracted with DCM (3×100 mL). The combined organic layers were washed with brine (3×100 mL), dried over Na₂SO₄, filtered, and concentrated under reduced pressure to afford the desired product as a solid. LCMS (ESI) m/z [M+H] calcd for C₄₂H₅₇F₃N₈O₆ 827.45; found: 827.5.

### Intermediate F38: Synthesis of (6³S,4S)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione

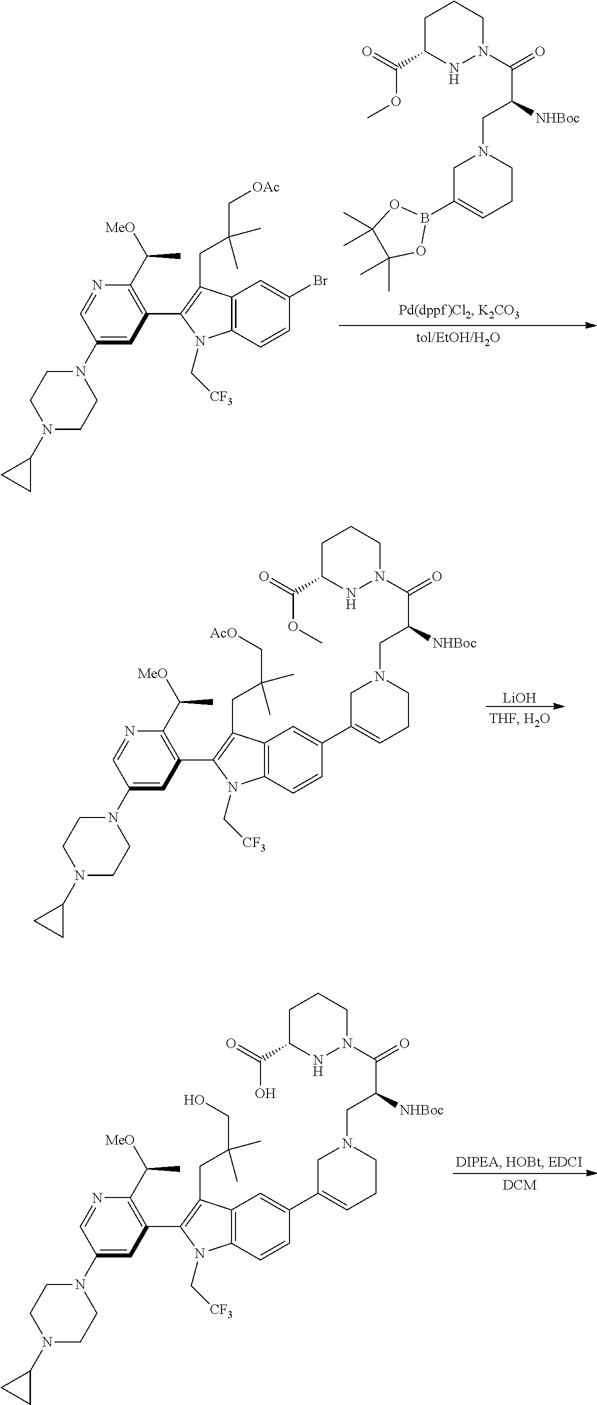

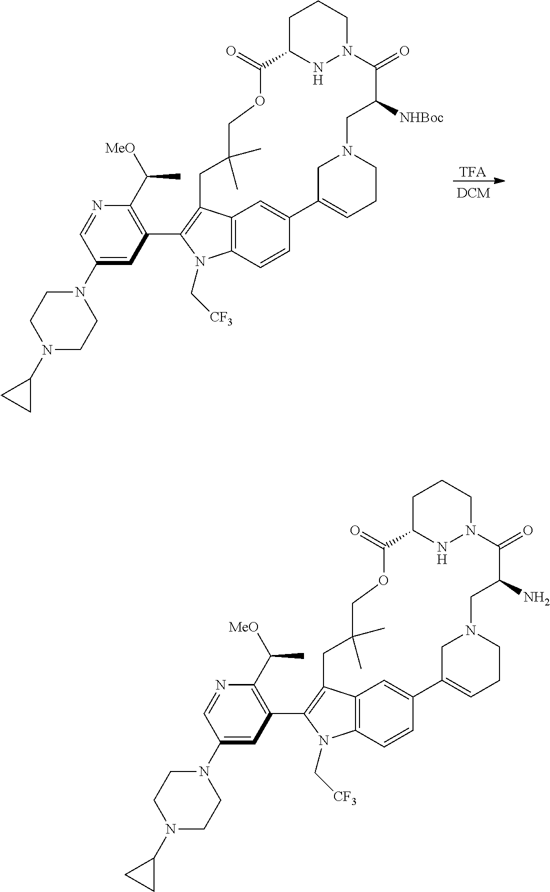

### Step 1: Synthesis of methyl (S)-1-((S)-3-(5-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)-3,6-dihydropyridin-1(2H)-yl)-2-((tert-butoxycarbonyl)amino)propanoyl)hexahydropyridazine-3-carboxylate

To a solution of (S)-3-(5-bromo-2-(5-(4-cyclopropylpiperazin-1-yl)-2-(1-methoxyethyl)pyridin-3-yl)-1-(2,2,2-trifluoroethyl)-1H-indol-3-yl)-2,2-dimethylpropyl acetate (9 g, 13.522 mmol), methyl (S)-1-((S)-2-((tert-butoxycarbonyl)amino)-3-(5-(4,4,5,5-tetramethyl-1,3,2-dioxaborolan-2-yl)-3,6-dihydropyridin-1(2H)-yl)propanoyl)hexahydropyridazine-3-carboxylate (7.14 g, 16.226 mmol), and K₂CO₃ (8.41 g, 60.849 mmol) in toluene (90 mL), dioxane (60 mL), and H₂O (30 mL) was added and Pd(dtbpf)Cl₂ (2.97 g, 4.057 mmol). The resulting mixture was stirred at 70° C. for 3 h and then cold H₂O (1 L). The resulting mixture was extracted with DCM (3×500 mL) and the combined organic layers were washed with brine (3×300 mL), dried over Na₂SO₄, filtered, and concentrated under reduced pressure. The residue was purified by silica gel column chromatography (5% MeOH/DCM) to afford the desired product (9 g, 67% yield) as a solid. LCMS (ESI) m/z [M+H] calcd for C₅₁H₇₁F₃N₈O₈: 981.54; found: 981.3.

### Step 2: Synthesis of (S)-1-((S)-2-((tert-butoxycarbonyl)amino)-3-(5-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)-3,6-dihydropyridin-1(2H)-yl)propanoyl)hexahydropyridazine-3-carboxylic acid

To a solution of methyl (S)-1-((S)-3-(5-(3-(3-acetoxy-2,2-dimethylpropyl)-2-(5-(4-cyclopropylpiperazin-1-yl)-2-((S)-1-methoxyethyl)pyridin-3-yl)-1-(2,2,2-trifluoroethyl)-1H-indol-5-yl)-3,6-dihydropyridin-1(2H)-yl)-2-((tert-butoxycarbonyl)amino)propanoyl)hexahydropyridazine-3-carboxylate (9 g, 9.173 mmol) in THE (70 mL) and H₂O (50 mL) at 0° C. was added LiOH⋅H₂O (0.88 g, 36.692 mmol). The resulting mixture was stirred overnight at room temperature and was then neutralized to pH 7 with HCl (aq.). The resulting mixture was extracted with DCM (3×200 mL) and the combined organic layers were washed with brine (3×200 mL), dried over Na₂SO₄, filtered, and concentrated under reduced pressure to afford the desired product (7.5 g, 88% yield) as a solid. LCMS (ESI) m/z [M+H] calcd for C₄₈H₆₇F₃N₈O₇: 925.52; found: 925.6.

### Step 3: Synthesis of tert-butyl ((63S,4S)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-2¹ 2²,2³,2⁶,6¹,6²,6³,6⁴,6⁵,6⁶-decahydro-1′H-8-oxa-1(5,3)-indola-6(1,3)-pyridazina-2(5,1)-pyridinacycloundecaphane-4-yl)carbamate

To a solution of (S)-1-((S)-2-((tert-butoxycarbonyl)amino)-3-(5-(2-(5-(4-cyclopropylpiperazin-1-yl)-2-((S)-1-methoxyethyl)pyridin-3-yl)-3-(3-hydroxy-2,2-dimethylpropyl)-1-(2,2,2-trifluoroethyl)-1H-indol-5-yl)-3,6-dihydropyridin-1(2H)-yl)propanoyl)hexahydropyridazine-3-carboxylic acid (7.5 g, 8.107 mmol) and DIPEA (41.91 g, 324.280 mmol) in DCM (750 mL) at 0° C. was added EDCI (46.62 g, 243.210 mmol) and HOBt (16.57 g, 48.642 mmol). The resulting mixture was stirred overnight at room temperature and then cold H₂O (1 L) was added. The resulting mixture was extracted with DCM (3×500 mL) and the combined organic layers were washed with brine (3×500 mL), dried over Na₂SO₄, filtered, and concentrated under reduced pressure. The residue was purified by silica gel column chromatography (20% EtOAc/pet. ether) to afford the desired product (6 g, 73% yield) as a solid. LCMS (ESI) m/z [M+H] calcd for C₄₈H₆₅F₃N₈O₆: 907.51; found: 907.6

### Step 4: Synthesis of (6³S,4S)-4-amino-1²-(5-(4-cyclopropylpiperazin-1-yl)-2-((S)-1-methoxyethyl)pyridin-3-yl)-10,10-dimethyl-1¹-(2,2,2-trifluoroethyl)-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione

To a solution of tert-butyl ((63S,4S)-1²-(5-(4-cyclopropylpiperazin-1-yl)-2-((S)-1-methoxyethyl)pyridin-3-yl)-10,10-dimethyl-5,7-dioxo-1¹-(2,2,2-trifluoroethyl)-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)carbamate (2 g, 2.205 mmol) in DCM (15 mL) at 0° C. was added TFA (15 mL). The resulting mixture was stirred at 0° C. for 1 h and then the mixture was basified to pH 8 with sat. NaHCO₃ (aq.). The resulting mixture was extracted with DCM (3×100 mL) and the combined organic layers were washed with brine (3×100 mL), dried over Na₂SO₄, filtered, and concentrated under reduced pressure to afford the desired product (1.65 g, 83% yield) as a solid. LCMS (ESI) m/z [M+H] calcd for C₄₃H₅₇F₃N₈O₄: 807.46; found: 807.7.

### Intermediate F39: Synthesis of (6³S,4S,Z)-4-amino-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

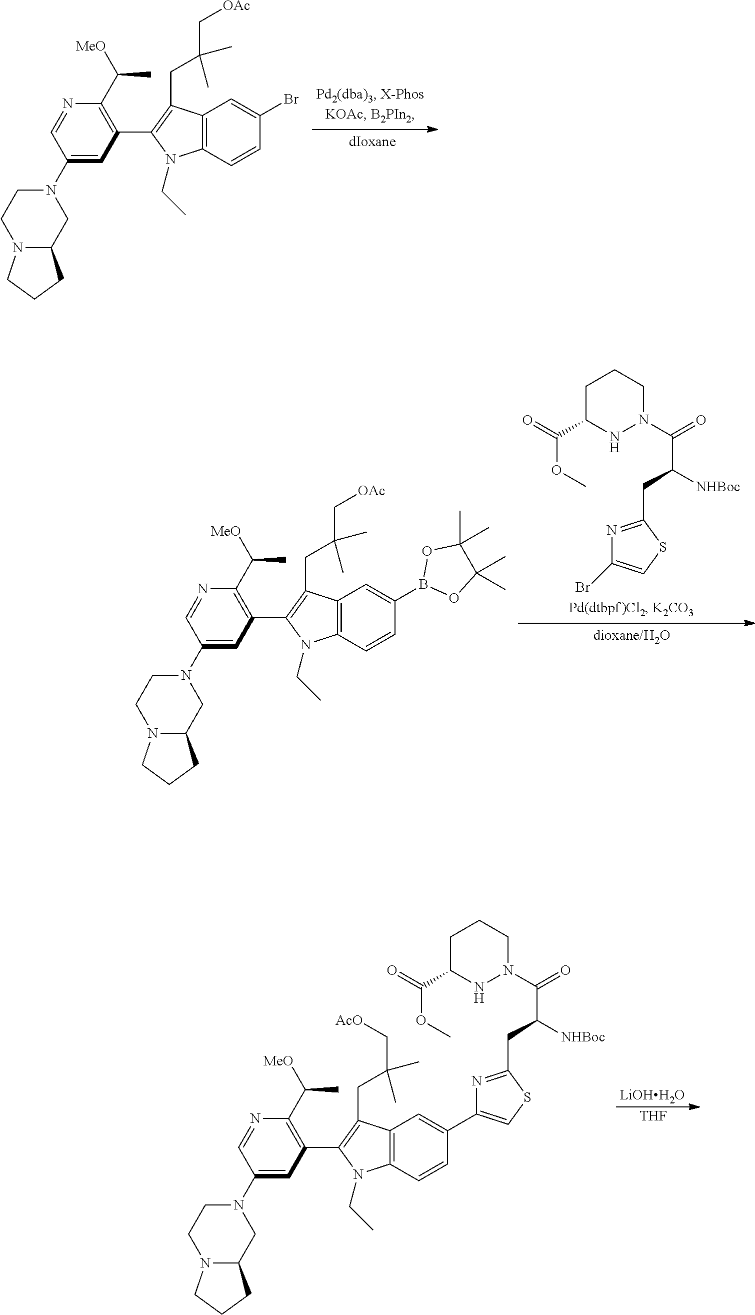

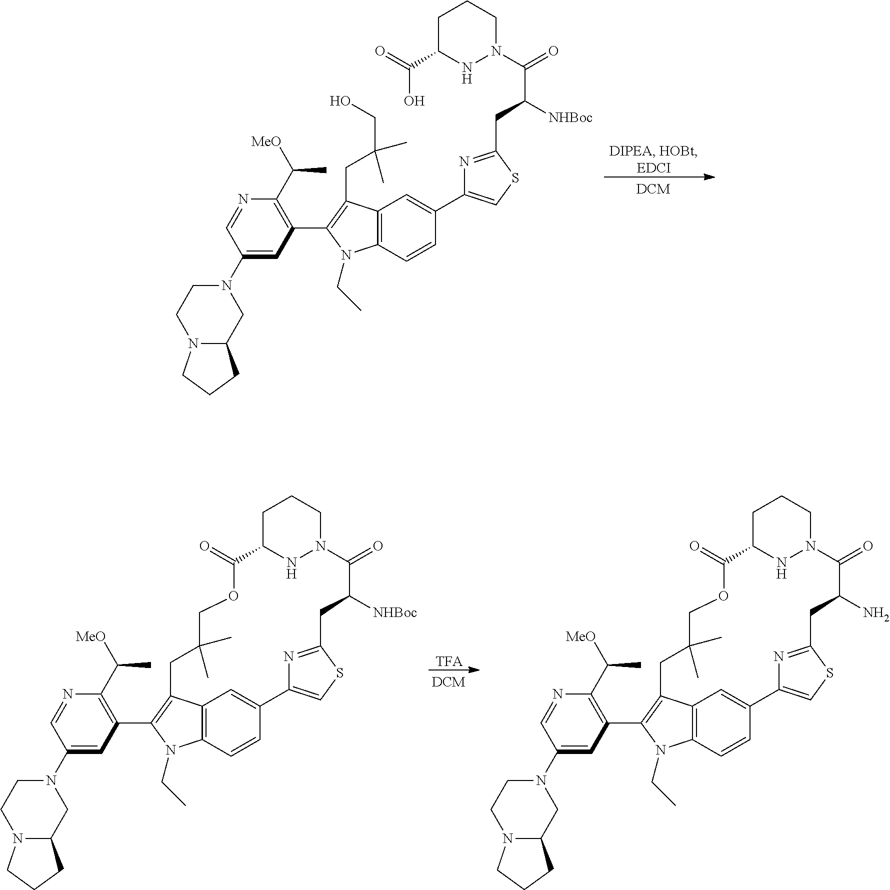

### Step 1: Synthesis of 3-(1-ethyl-2-(5-((R)-hexahydropyrrolo[1,2-a]pyrazin-2(1H)-yl)-2-((S)-1-methoxyethyl)pyridin-3-yl)-1H-indol-3-yl)-2,2-dimethylpropyl acetate

To a solution of 3-5(5-bromo-1-ethyl-2-(5-((R)-hexahydropyrrolo[1,2-a]pyrazin-2(1H)-yl-2-((S)-1-methoxyethyl)pyridin-3-yl)-1H-indol-3-yl)-2,2-dimethylpropyl acetate (7 g, 11.445 mmol), KOAc (2.81 g, 28.613 mmol) and 4,4,5,5-tetramethyl-2-(tetra methyl-1,3,2-dioxaborolan-2-yl)-1,3,2-dioxaborolane (5.81 g, 22.890 mmol) in dioxane (70 mL) was added Pd₂(dba)₃ (2.10 g, 2.289 mmol) and X-Phos (2.18 g, 4.578 mmol). The resulting mixture was stirred for 3 h at 80° C. and then cold H₂O (50 mL) was added. The aqueous layer was extracted with EtOAc (3×30 mL), and the combined organic layers were washed with brine, dried over Na₂SO₄, filtered, and concentrated under reduced pressure. The residue was purified by column chromatography (10% MeOH/DCM) to afford the desired product (5.2 g, 69% yield) as a solid. LCMS (ESI) m/z [M+H] calcd for C₃₈H₅₅BN₄O₅ 659.44, found: 659.7.

### Step 2: Synthesis of methyl (S)-1-((S)-3-(4-(3-(3-acetoxy-2,2-dimethylpropyl)-1-ethyl-2-(5-((R)-hexahydropyrrolo[1,2-a]pyrazin-2(1H)-yl)-2-((S)-1-methoxyethyl)pyridin-3-yl)-1H-indol-5-yl)thiazol-2-yl)-2-((tert-butoxycarbonyl)amino)propanoyl)hexahydropyridazine-3-carboxylate

To a stirred mixture of 3-(1-ethyl-2-(5-((R)-hexahydropyrrolo[1,2-a]pyrazin-2(1H)-yl)-2-((S)-1-methoxyethyl)pyridin-3-yl)-5-(4,4,5,5-tetramethyl-1,3,2-dioxaborolan-2-yl)-1H-indol-3-yl)-2,2-dimethylpropyl acetate (5.2 g, 7.894 mmol), methyl (S)-1-((S)-3-(4-bromothiazol-2-yl)-2-((tert-butoxycarbonyl)amino)propanoyl)hexahydropyridazine-3-carboxylate (47.11 mg, 0.099 mmol), and K₂CO₃ (2.73 g, 19.735 mmol) in dioxane (52 mL) and H₂O (11 mL) was added Pd(dtbpf)Cl₂ (0.51 g, 0.789 mmol). The resulting mixture was stirred at 70° C. for 2 h and then H₂O was added. The aqueous layer was extracted with EtOAc (3×50 mL), and the combined organic layers were washed with brine, dried over Na₂SO₄, filtered, and concentrated under reduced pressure. The residue was purified by column chromatography (10% MeOH/DCM) to afford the desired product (3 g, 74% yield) as a solid. LCMS (ESI) m/z [M+H] calcd for C₄₉H₆₈N₈O₈S 929.50; found: 930.1.

### Step 3: Synthesis of (S)-1-((S)-2-((tert-butoxycarbonyl)amino)-3-(4-(1-ethyl-2-(5-((R)-hexahydropyrrolo[1,2-a]pyrazin-2(1H)-yl)-2-((S)-1-methoxyethyl)pyridin-3-yl)-3-(3-hydroxy-2,2-dimethylpropyl)-1H-indol-5-yl)thiazol-2-yl)propanoyl)hexahydropyridazine-3-carboxylic acid

To a solution of methyl (S)-1-((S)-3-(4-(3-(3-acetoxy-2,2-dimethylpropyl)-1-ethyl-2-(5-((R)-hexahydropyrrolo[1,2-a]pyrazin-2(1H)-yl)-2-((S)-1-methoxyethyl)pyridin-3-yl)-1H-indol-5-yl)thiazol-2-yl)-2-((tert-butoxycarbonyl)amino)propanoyl)hexahydropyridazine-3-carboxylate (3 g, 3.2501 mmol) in THE (30 mL) at 0° C. was added LiOH⋅H₂O (0.55 g, 13.012 mmol). The resulting mixture was stirred overnight at room temperature. The mixture was neutralized to pH 7 with HCl (aq). The resulting mixture was extracted with EtOAc (3×50 mL). The combined organic layers were washed with brine (3×50 mL), dried over anhydrous Na₂SO₄, filtered, and concentrated under reduced pressure to afford the product (2.96 g, 92% yield) as a solid. LCMS (ESI) m/z [M+H] calcd for C₄₆H₆₄N₈O₇S: 873.47; found: 873.6.

### Step 4: Synthesis of tert-butyl ((6³S,4S,Z)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate

To a solution of (S)-1-((S)-2-((tert-butoxycarbonyl)amino)-3-(4-(1-ethyl-2-(5-((R)-hexahydropyrrolo[1,2-a]pyrazin-2(1H)-yl)-2-((S)-1-methoxyethyl)pyridin-3-yl)-3-(3-hydroxy-2,2-dimethylpropyl)-1H-indol-5-yl)thiazol-2-yl)propanoyl)hexahydropyridazine-3-carboxylic acid (2.9 g, 3.348 mmol), HOBt (2.26 g, 16.740 mmol) and DIPEA (8.65 g, 66.960 mmol) in DCM (30 mL) at 0° C. was added EDCI (6.42 g, 33.480 mmol) under an argon atmosphere. The resulting mixture was stirred overnight at room temperature and then cold H₂O (40 mL) was added. The mixture was extracted with DCM (3×20 mL) and the combined organic layers were washed with brine (3×20 mL), dried over Na₂SO₄, filtered, and concentrated under reduced pressure. The residue was purified by silica gel column chromatography (10% MeOH/DCM) to afford the desired product (900 mg, 37% yield) as a solid. LCMS (ESI) m/z [M+H] calcd for C₄₆H₆₂N₈O₆S: 855.46; found: 855.1

### Step 5: Synthesis of (6³S,4S,Z)-4-amino-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione

To a solution of tert-butyl ((63S,4S,2)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)carbamate (900 mg, 1.052 mmol) in DCM (8 mL) at 0° C. was added TFA (4 mL). The resulting mixture was stirred at 0° C. for 3 h. The mixture was basified to pH 8 with sat. aq. NaHCO₃. The resulting mixture was extracted with DCM (3×50 mL). The combined organic layers were washed with brine (3×50 mL), dried over Na₂SO₄, filtered, and concentrated under reduced pressure to afford the desired product (850 mg, 81% yield) as a solid. LCMS (ESI) m/z [M+H] calcd for C₄₁H₅₄N₈O₄S: 755.41; found: 755.5.

### Intermediate A-1 and A-2: Synthesis of (S)-2-((R)-7-(tert-butoxycarbonyl)-2,7-diazaspiro[4.4]nonan-2-yl)-2-cyclopentylacetic acid and (S)-2-((S)-7-(tert-butoxycarbonyl)-2,7-diazaspiro[4.4]nonan-2-yl)-2-cyclopentylacetic acid

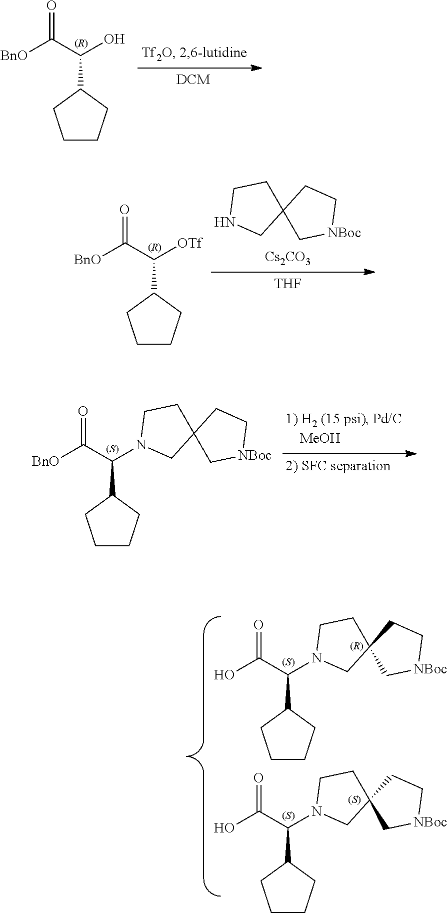

### Step 1: Synthesis of benzyl (R)-2-cyclopentyl-2-(((trifluoromethyl)sulfonyl)oxy)acetate PUP-3776, C₃

To a solution of benzyl (R)-2-cyclopentyl-2-hydroxyacetate (3 g, 12.8 mmol) in DCM (50 mL) was added Tf₂O (3.79 g, 13.44 mmol) and 2,6-lutidine (1.51 g, 14.09 mmol) at 0° C. under N₂ and the mixture was stirred at 0° C. for 2 h. The residue was diluted with H₂O (30 mL) and extracted with DCM (3×50 mL). The combined organic layers were washed with brine (2×50 mL), dried over Na₂SO₄, filtered and the filtrate was concentrated under reduced pressure to give the product, which was used directly without further purification.

### Step 2: Synthesis of tert-butyl 7-((S)-2-(benzyloxy)-1-cyclopentyl-2-oxoethyl)-2,7-diazaspiro[4.4]nonane-2-carboxylate

To a solution of benzyl (R)-2-cyclopentyl-2-(((trifluoromethyl)sulfonyl)oxy)acetate (4.86 g, 13.26 mmol) in THE (20 mL) was added tert-butyl 2,7-diazaspiro[4.4]nonane-2-carboxylate (2 g, 8.84 mmol) and Cs₂CO₃ (8.64 g, 26.51 mmol). The mixture was stirred at room temperature for 30 min. The residue was diluted with H₂O (30 mL) and extracted with EtOAc (3×30 mL) and the combined organic layers were washed with brine (2×40 mL), dried over Na₂SO₄, filtered, and concentrated under reduced pressure. The residue was purified by silica gel column chromatography (20→100% EtOAc/pet. ether) to give the product (2.6 g, 66% yield) as a solid. LCMS (ESI) m/z [M+H] calcd for C₂₆H₃₈N₂O₄:443.3; found: 443.2.

### Step 3: Synthesis of (S)-2-((R)-7-(tert-butoxycarbonyl)-2,7-diazaspiro[4.4]nonan-2-yl)-2-cyclopentylacetic acid and (S)-2-((S)-7-(tert-butoxycarbonyl)-2,7-diazaspiro[4.4]nonan-2-yl)-2-cyclopentylacetic acid

To a solution of tert-butyl 7-((S)-2-(benzyloxy)-1-cyclopentyl-2-oxoethyl)-2,7-diazaspiro[4.4]nonane-2-carboxylate (2.6 g, 5.87 mmol) in MeOH (30 mL) was added Pd/C (0.5 g, 10% on carbon w/w) under a N₂ atmosphere. The suspension was degassed and purged with H₂. The mixture was stirred under H₂ (15 psi) at room temperature for 4 h. The mixture was filtered and the filtrate was concentrated under reduced pressure to give residue. The residue was dissolved in EtOAc (5 mL) and the mixture was stirred for 10 min. Then the mixture was filtered and the filter cake was dried under reduced pressure. The solid was purified by SFC-separation (CO₂/MeOH (0.1% NH₄O)) to give (S)-2-((R)-7-(tert-butoxycarbonyl)-2,7-diazaspiro[4.4]nonan-2-yl)-2-cyclopentylacetic acid (450 mg, 22% yield) and (S)-2-((S)-7-(tert-butoxycarbonyl)-2,7-diazaspiro[4.4]nonan-2-yl)-2-cyclopentylacetic acid (450 mg, 22% yield).

### Intermediate A-3 and A-4: (S)-2-((S)-7-(tert-butoxycarbonyl)-2,7-diazaspiro[4.4]nonan-2-yl)-3-methylbutanoic acid and (S)-2-((R)-7-(tert-butoxycarbonyl)-2,7 diazaspiro[4.4]nonan-2-yl)-3-methylbutanoic acid

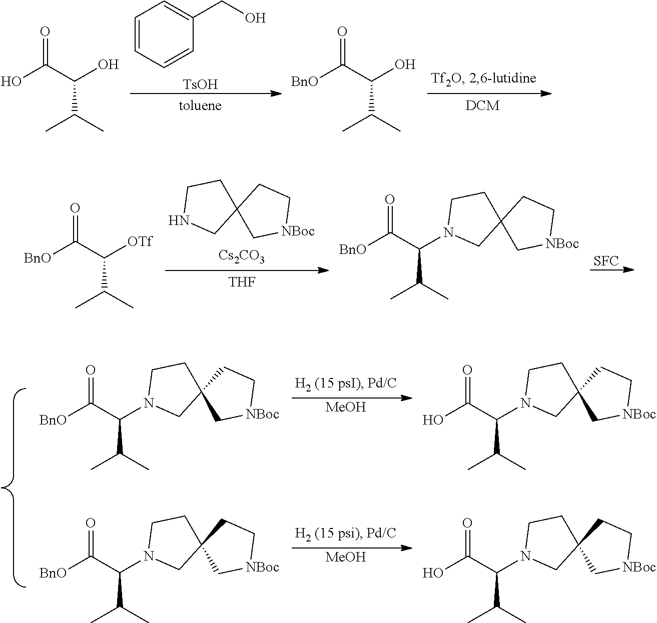

### Step 1: Synthesis of benzyl (R)-2-hydroxy-3-methylbutanoate

To a stirred solution of benzyl alcohol (19.22 g, 177.77 mmol) in toluene (200 mL) was added TsOH⋅H₂O (2.92 g, 16.93 mmol) in portions at room temperature under N₂. The mixture was stirred at 80° C. for 30 min, the mixture then cooled to room temperature and (R)-2-hydroxy-3-methylbutanoic acid (20 g, 169.30 mmol, 1 eq) was added. The resultant mixture was stirred at 80° C. for 3 h. The reaction mixture was concentrated under reduced pressure to give the crude product as colorless oil. The crude product was purified by silica gel column chromatography (20→100% EtOAc/pet. ether) to afford the product (25 g, 71% yield) as an oil.

### Step 2: Synthesis of benzyl (R)-3-methyl-2-(((trifluoromethyl)sulfonyl)oxy)butanoate

A solution of benzyl (R)-2-hydroxy-3-methylbutanoate (15 g, 72.03 mmol) in DCM (225 mL) was cooled to 0° C. and then treated with Tf₂O (21.34 g, 75.63 mmol) and 2,6-lutidine (8.49 g, 79.23 mmol) under N₂. The resultant mixture was stirred for 1 h at 0° C. The reaction mixture was added into H₂O (300 mL). The mixture was extracted with DCM (3×100 mL) and the combined organic layers were washed with brine (400 mL), filtered and concentrated under reduced pressure to give the crude product. The crude product was purified by silica gel column chromatography (5→10% EtOAc/pet. ether: EtOAc) to afford the product (20 g, 82% yield) as an oil.

### Step 3: Synthesis of tert-butyl 7-((S)-1-(benzyloxy)-3-methyl-1-oxobutan-2-yl)-2,7-diazaspiro [4.4]nonane-2-carboxylate

To a solution of benzyl (R)-3-methyl-2-(((trifluoromethyl)sulfonyl)oxy)butanoate (20 g, 58.77 mmol) and tert-butyl 2,7-diazaspiro[4.4]nonane-2-carboxylate (11.08 g, 48.97 mmol), Cs₂CO₃ (47.87 g, 146.92 mmol) in THE (300 mL) at 0° C. The resultant mixture was stirred for 2 h at room temperature. The reaction mixture was filtered and the filter cake washed with THE (3×100 mL). Then the filtrate was concentrated under reduced pressure to give the crude product as an oil. The oil was purified by silica gel column chromatography (10→30% EtOAc/pet. ether) to give the product (13.2 g, 64% yield). LCMS (ESI) m/z [M+H] calcd for C₂₄H₃₇N₂O₄: 417.27; found: 417.2

### Step 4: Synthesis tert-butyl (S)-7-((S)-1-(benzyloxy)-3-methyl-1-oxobutan-2-yl)-2,7-diazaspiro[4.4]nonane-2-carboxylate and tert-butyl (R)-7-((S)-1-(benzyloxy)-3-methyl-1-oxobutan-2-yl)-2,7-diazaspiro[4.4]nonane-2-carboxylate

The tert-butyl 7-((S)-1-(benzyloxy)-3-methyl-1-oxobutan-2-yl)-2,7-diazaspiro[4.4]nonane-2-carboxylate (10 g) was purified by SFC separation to afford tert-butyl (S)-7-((S)-1-(benzyloxy)-3-methyl-1-oxobutan-2-yl)-2,7-diazaspiro[4.4]nonane-2-carboxylate (3.4 g) and tert-butyl (R)-7-((S)-1-(benzyloxy)-3-methyl-1-oxobutan-2-yl)-2,7-diazaspiro[4.4]nonane-2-carboxylate (3.9 g).

### Step 5: Synthesis of (S)-2-((R)-7-(tert-butoxycarbonyl)-2,7-diazaspiro[4.4]nonan-2-yl)-3-methylbutanoic acid

To a solution of tert-butyl (R)-7-((S)-1-(benzyloxy)-3-methyl-1-oxobutan-2-yl)-2,7-diazaspiro[4.4]nonane-2-carboxylate (2.5 g, 6.00 mmol) in MeOH (25 mL) was added Pd/C (1.5 g, 10% purity) under Ar. The suspension was degassed under reduced pressure and purged with H₂ several times. The mixture was stirred under H₂ (15 psi) at room temperature for 1 h. The reaction mixture was filtered and then the filtrate was concentrated under reduced pressure to give the product (1.9 g, crude) as a solid.

### Step 6: Synthesis of (S)-2-((S)-7-(tert-butoxycarbonyl)-2,7-diazaspiro[4.4]nonan-2-yl)-3-methylbutanoic acid

To a solution of tert-butyl (S)-7-((S)-1-(benzyloxy)-3-methyl-1-oxobutan-2-yl)-2,7-diazaspiro[4.4]nonane-2-carboxylate (3 g, 7.20 mmol) in MeOH (5 mL) was added Pd/C (1 g, 10% purity) under Ar. The suspension was degassed under reduced pressure and purged with H₂ several times. The mixture was stirred under H₂ (15 psi) at room temperature for 1 h. The reaction mixture was filtered and then the filtrate was concentrated under reduced pressure to give the product (2.3 g, 98% yield) as solid.

### Intermediate A-5 and A-6: Synthesis of tert-butyl (R)-7-((S)-1-(benzyloxy)-3-methyl-1-oxobutan-2-yl)-6-oxo-2,7-diazaspiro[4.4]nonane-2-carboxylate and tert-butyl (S)-7-((S)-1-(benzyloxy)-3-methyl-1-oxobutan-2-yl)-6-oxo-2,7-diazaspiro[4.4]nonane-2-carboxylate carboxylate

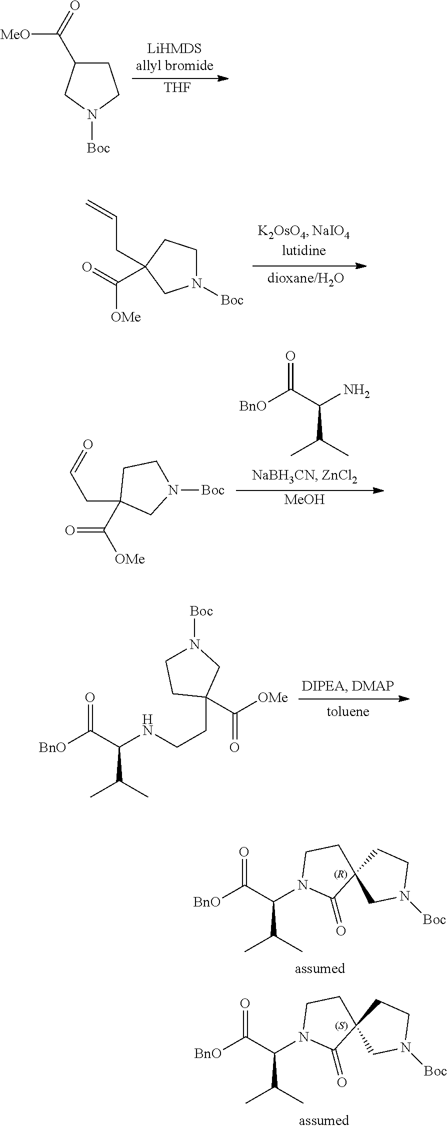

### Step 1: Synthesis of 1-(tert-butyl) 3-methyl 3-allylpyrrolidine-1,3-dicarboxylate

To a mixture of 1-(tert-butyl) 3-methyl pyrrolidine-1,3-dicarboxylate (10 g, 43.616 mmol) in THE (100 mL) at −78° C. was added 1M LiHMDS (65.42 mL, 65.424 mmol), dropwise. The resulting mixture was stirred at −78° C. for 1 h and then a solution of allyl bromide (7.91 g, 65.423 mmol) in THE was added dropwise over 10 min. The resulting mixture was stirred at −78° C. for an additional 2 h and was then quenched by the addition of sat. aq. NH₄Cl at 0° C. The resulting mixture was extracted with EtOAc (3×100 mL) and the combined organic layers were washed with brine (2×80 mL), dried with Na₂SO₄, filtered, and concentrated under reduced pressure. Purification by silica gel column chromatography (20% EtOAc/pet. ether) afforded the desired product (10 g, 76% yield).

### Step 2: Synthesis of 1-(tert-butyl) 3-methyl 3-(2-oxoethyl)pyrrolidine-1,3-dicarboxylate

To a mixture of 1-(tert-butyl) 3-methyl 3-allylpyrrolidine-1,3-dicarboxylate (11.0 g, 40.84 mmol) and 2,6-lutidine (8.75 g, 81.68 mmol) in dioxane (190 mL) and H₂O (19 mL) at 0° C. was added K₂OsO₄⋅2H₂O (0.75 g, 2.04 mmol). The resulting mixture was stirred at 0° C. for 15 min and then NaIO₄ (34.94 g, 163.36 mmol) was added in portions. The mixture was warmed to room temperature and stirred for an additional 3 h, then was quenched by the addition of sat. aq. Na₂S₂O₃ at 0° C. The resulting mixture was extracted with EtOAc (3×300 mL) and the combined organic layers were washed with brine (200 mL), dried with Na₂SO₄, filtered, and concentrated under reduced pressure. Purification by reverse phase chromatography (0→40% MeCN/H₂O, 0.1% HCO₂H) afforded the desired product (6.4 g, 51% yield).

### Step 3: Synthesis of 1-(tert-butyl) 3-methyl 3-(2-(((S)-1-(benzyloxy)-3-methyl-1-oxobutan-2-yl)amino)ethyl)pyrrolidine-1,3-dicarboxylate

To a mixture of 1-(tert-butyl) 3-methyl 3-(2-oxoethyl)pyrrolidine-1,3-dicarboxylate (6.30 g, 23.220 mmol) and benzyl L-valinate (7.22 g, 34.831 mmol) in MeOH (70 mL) at 0° C. was added ZnCl₂ (4.75 g, 34.831 mmol). The resulting mixture was warmed to room temperature and stirred for 30 min, then cooled to 0° C. NaBH₃CN (2.92 g, 46.441 mmol) was added in portions then the mixture was warmed to room temperature and stirred for 2 h. The reaction was quenched by the addition of sat. aq. NH₄Cl at 0° C. and the resulting mixture was then extracted with EtOAc (3×200 mL). The combined organic layers were washed with brine (150 mL), dried with Na₂SO₄, filtered, and concentrated under reduced pressure. Purification by silica gel column chromatography (33% EtOAc/pet. ether) afforded the desired product (6.4 g, 53% yield). LCMS (ESI) m/z [M+H] calcd for C₂₅H₃₈N₂O₆: 463.28; found: 463.3.

### Step 4: Synthesis of tert-butyl (R)-7-((S)-1-(benzyloxy)-3-methyl-1-oxobutan-2-yl)-6-oxo-2,7-diazaspiro[4.4]nonane-2-carboxylate and tert-butyl (S)-7-((S)-1-(benzyloxy)-3-methyl-1-oxobutan-2-yl)-6-oxo-2,7-diazaspiro[4.4]nonane-2-carboxylate

To a mixture of 1-(tert-butyl) 3-methyl 3-(2-(((S)-1-(benzyloxy)-3-methyl-1-oxobutan-2-yl)amino)ethyl)pyrrolidine-1,3-dicarboxylate (4.50 g, 9.728 mmol) and DIPEA (16.6 mL, 97.28 mmol) in toluene (50 mL) was added DMAP (1.19 g, 9.728 mmol) and then the mixture was heated to 80° C. After 24 h the reaction was cooled to room temperature and concentrated under reduced pressure, and the residue was purified by reverse phase chromatography (10→50% MeCN/H₂O, 0.1% HCO₂H). The diastereomers were then separated by chiral prep-SFC (30% EtOH/CO₂) to afford tert-butyl (R)-7-((S)-1-(benzyloxy)-3-methyl-1-oxobutan-2-yl)-6-oxo-2,7-diazaspiro[4.4]nonane-2-carboxylate (1.0 g, 32% yield, LCMS (ESI) m/z [M+H] calcd for C₂₄H₃₄N₂O₅: 431.26; found: 431.2) and tert-butyl (S)-7-((S)-1-(benzyloxy)-3-methyl-1-oxobutan-2-yl)-6-oxo-2,7-diazaspiro[4.4]nonane-2-carboxylate (1.0 g, 32% yield, LCMS (ESI) m/z [M+H] calcd for C₂₄H₃₄N₂O₅: 431.26; found: 431.2).

### Intermediate A-7: Synthesis of (S)-2-((R)-7-(tert-butoxycarbonyl)-1-oxo-2,7-diazaspiro[4.4]nonan-2-yl)-3-methylbutanoic acid

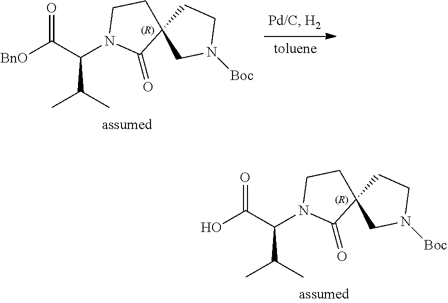

To a solution of tert-butyl (R)-7-((S)-1-(benzyloxy)-3-methyl-1-oxobutan-2-yl)-6-oxo-2,7-diazaspiro[4.4]nonane-2-carboxylate (600 mg, 1.4 mmol) in toluene (20 mL) was added Pd/C (120 mg, 1.1 mmol). The reaction mixture was heated at 50° C. and stirred under a hydrogen atmosphere (1 atm) for 3 h. The mixture was filtered, and the filter cake was washed with MeOH (3×20 mL). The filtrate was concentrated under reduced pressure to afford the desired product (550 mg, crude). LCMS (ESI) m/z [M−H] calcd for C₁₇H₂₈N₂O₅: 339.19; found: 339.1.

### Intermediate A-8: Synthesis of (S)-2-((S)-7-(tert-butoxycarbonyl)-1-oxo-2,7-diazaspiro[4.4]nonan-2-yl)-3-methylbutanoic acid

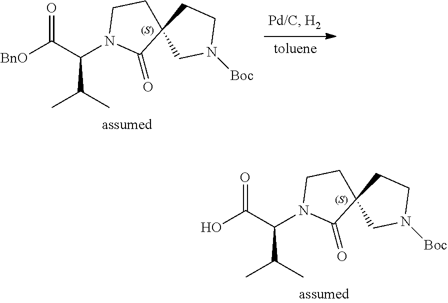

To a solution of tert-butyl (S)-7-((S)-1-(benzyloxy)-3-methyl-1-oxobutan-2-yl)-6-oxo-2,7-diazaspiro[4.4]nonane-2-carboxylate (550 mg, 1.3 mmol) in toluene (30 mL) was added Pd/C (120 mg, 1.1 mmol). The reaction mixture was heated at 50° C. and stirred under a hydrogen atmosphere (1 atm) for 3 h. The mixture was filtered, and the filter cake was washed with MeOH (3×20 mL). The filtrate was concentrated under reduced pressure to afford the desired product (550 mg, crude). LCMS (ESI) m/z [M−H] calcd for C₁₇H₂₈N₂O₅: 339.19; found: 339.2.

### Intermediates A-9 and A-10: Synthesis of Tert-Butyl (R)-7-((S)-2-(benzyloxy)-1-cyclopentyl-2-oxoethyl)-6-oxo-2,7-diazaspiro[4.4]nonane-2-carboxylate and tert-butyl (S)-7-((S)-2-(benzyloxy)-1-cyclopentyl-2-oxoethyl)-6-oxo-2,7-diazaspiro[4.4]nonane-2-carboxylate

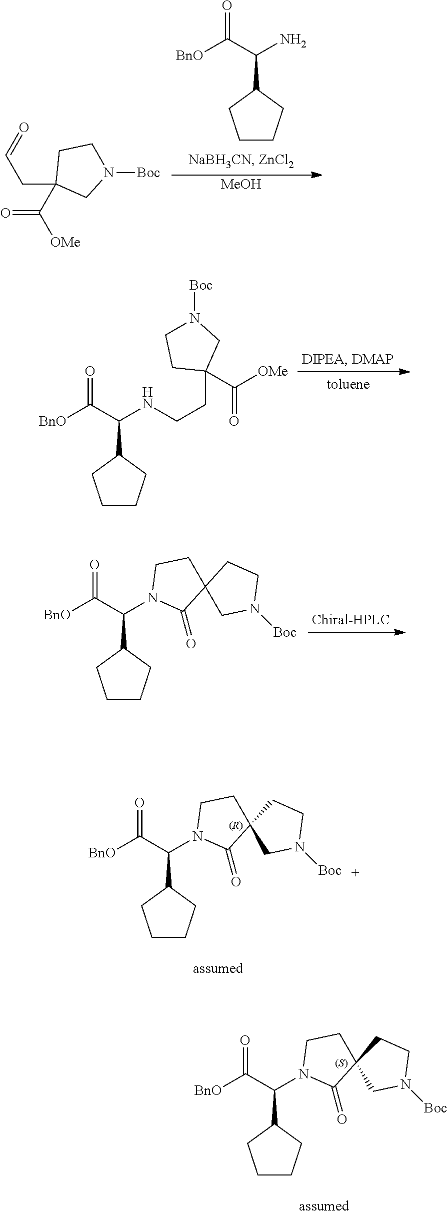

### Step 1: Synthesis of 1-(tert-butyl) 3-methyl 3-(2-(((S)-2-(benzyloxy)-1-cyclopentyl-2-oxoethyl)amino)ethyl)pyrrolidine-1,3-dicarboxylate

To a solution of 1-(tert-butyl) 3-methyl 3-(2-oxoethyl)pyrrolidine-1,3-dicarboxylate (9.60 g, 35.4 mmol) in MeOH (100 mL) at 0° C. was added benzyl (S)-2-amino-2-cyclopentylacetate (12.38 g, 53.075 mmol) and zinc chloride (7.23 g, 53.1 mmol). After 30 min NaBH₃CN (4.45 g, 70.8 mmol) was added and the resulting mixture stirred for 2 h at room temperature, concentrated under reduced pressure and the residue diluted with H₂O (150 mL). The aqueous layer was extracted with EtOAc (3×50 mL) and the combined organic layers were washed with brine, dried with Na₂SO₄, filtered, and then concentrated under reduced pressure. Purification by silica gel column chromatography (20% EtOAc/pet. ether) afforded the desired product (11.1 g, 64% yield). LCMS (ESI) m/z [M+H] calcd for C₂₇H₄₀N₂O₆: 489.30; found: 489.3.

### Step 2: Synthesis of tert-butyl (R)-7-((S)-2-(benzyloxy)-1-cyclopentyl-2-oxoethyl)-6-oxo-2,7-diazaspiro[4.4]nonane-2-carboxylate and tert-butyl (S)-7-((S)-2-(benzyloxy)-1-cyclopentyl-2-oxoethyl)-6-oxo-2,7-diazaspiro[4.4]nonane-2-carboxylate

To a solution of stirred solution of 1-(tert-butyl) 3-methyl 3-(2-(((S)-2-(benzyloxy)-1-cyclopentyl-2-oxoethyl)amino)ethyl)pyrrolidine-1,3-dicarboxylate (11.1 g, 22.7 mmol) in toluene (120 mL) was added DIPEA (39.6 mL, 227 mmol) and DMAP (2.78 g, 22.7 mmol). The resulting mixture was stirred for 2 days at 80° C. and then concentrated under reduced pressure. Purification by reverse phase chromatography (20→70% MeCN/H₂O, 0.1% HCO₂H) afforded a mixture of desired products. The diastereomers were separated by prep-SFC (30% EtOH/CO₂) to afford tert-butyl (R)-7-((S)-2-(benzyloxy)-1-cyclopentyl-2-oxoethyl)-6-oxo-2,7-diazaspiro[4.4]nonane-2-carboxylate (3.73 g, 44% yield) LCMS (ESI) m/z [M+H] calcd for C₂₆H₃₆N₂O₅: 457.27; found: 457.3 and tert-butyl (S)-7-((S)-2-(benzyloxy)-1-cyclopentyl-2-oxoethyl)-6-oxo-2,7-diazaspiro[4.4]nonane-2-carboxylate (3.87 g, 46% yield)) LCMS (ESI) m/z [M+H] calcd for C₂₆H₃₆N₂O₅: 457.27; found: 457.3.

### Intermediates A-11: Synthesis of (S)-2-(2-(tert-butoxycarbonyl)-2,6-diazaspiro[3.5]nonan-6-yl)-2-cyclopentylacetic acid

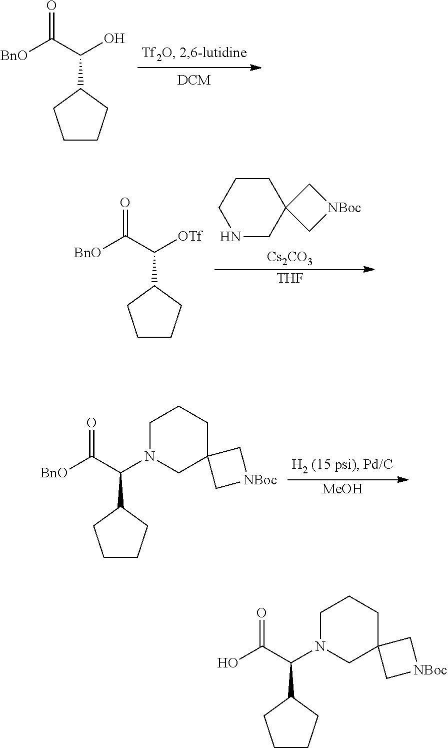

### Step 1: Synthesis of benzyl (R)-2-cyclopentyl-2-(((trifluoromethyl)sulfonyl)oxy)acetate

A solution of benzyl (R)-2-cyclopentyl-2-hydroxyacetate (500 mg, 2.13 mmol) in DCM (5 mL) was cooled to 0° C. and was treated with Tf₂O (632.22 mg, 2.24 mmol) and 2,6-lutidine (251.54 mg, 2.35 mmol) under N₂. The resultant mixture was stirred for 1 h at 0° C. The reaction mixture was added to H₂O (10 mL). Then the mixture was extracted with DCM (3×5 mL) and separated. The combined organic layers were washed with brine (10 mL), filtered and concentrated under reduced pressure to give the product (880 mg, crude) as brown oil.

### Step 2: Synthesis of tert-butyl (S)-6-(2-(benzyloxy)-1-cyclopentyl-2-oxoethyl)-2,6-diazaspiro[3.5]nonane-2-carboxylate

To a solution of benzyl (R)-2-cyclopentyl-2-(((trifluoromethyl)sulfonyl)oxy)acetate (880 mg, 2.11 mmol) and tert-butyl 2,6-diazaspiro[3.5]nonane-2-carboxylate (367.99 mg, 1.63 mmol) in THE (10 mL) at 0° C. was added Cs₂CO₃ (1.59 g, 4.88 mmol). The resulting mixture was stirred for 2 h at room temperature. The reaction mixture was filtered and the filter cake washed with THE (3×20 mL). Then the filtrate was concentrated under reduced pressure to give the crude product as colorless oil. The crude oil was purified by silica gel column chromatography (0→30% EtOAc/pet. ether) to give the product (505 mg, 70% yield) as an oil. LCMS (ESI) m/z [M+H] calcd for C₂₆H₃₉N₂O₄: 443.3; found: 443.3.

### Step 3: Synthesis of (S)-2-(2-(tert-butoxycarbonyl)-2,6-diazaspiro[3.5]nonan-6-yl)-2-cyclopentylacetic acid

To a solution of tert-butyl (S)-6-(2-(benzyloxy)-1-cyclopentyl-2-oxoethyl)-2,6-diazaspiro[3.5]nonane-2-carboxylate (500 mg, 1.13 mmol) in MeOH (10 mL) was added Pd/C (300 mg, 10% purity) under Ar. The suspension was degassed under reduced pressure and purged with H₂ several times. The mixture was stirred under H₂ (15 psi) at room temperature for 2 h. The suspension was filtered through a pad of Celite and the filter cake was washed with MeOH (5×30 mL). The combined filtrates were concentrated under reduced pressure to give the product (380 mg, 95% yield) as solid, which was used directly in the next step.

### Intermediate B-1: Synthesis of (2S,3S)-1-(tert-butylsulfinyl)-3-cyclopropylaziridine-2-carboxylic acid

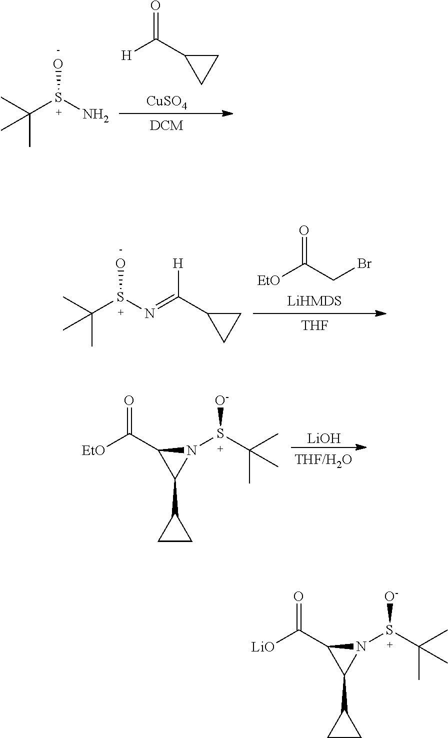

### Step 1: Synthesis of (E)-N-(cyclopropylmethylene)-2-methylpropane-2-sulfinamide

To a suspension of (S)-2-methylpropane-2-sulfinamide (4.0 g, 33.0 mmol) and CuSO₄ (15.80 g, 99.01 mmol) in DCM (200.0 mL) was added cyclopropanecarbaldehyde (4.63 g, 66.0 mmol). The resulting mixture was stirred overnight and was then filtered, the filter cake was washed with DCM (3×100 mL), and the filtrate was concentrated under reduced pressure to afford the desired product (3.5 g, 61% yield). LCMS (ESI) m/z [M+H] calcd for C₈H₁₅NOS: 174.10; found: 174.1.

### Step 2: Synthesis of ethyl (2S,3S)-1-(tert-butylsulfinyl)-3-cyclopropylaziridine-2-carboxylate

To a solution of ethyl bromoacetate (481.91 mg, 2.886 mmol) in THE (5.0 mL) at −78° C. was added LiHMDS (2.90 mL, 2.90 mmol). The resulting mixture was stirred for 2 h at −78° C. and then a solution of (E)-N-(cyclopropylmethylene)-2-methylpropane-2-sulfinamide (250.0 mg, 1.443 mmol) was added. The resulting mixture was stirred for 2 h at −78° C. and then quenched with H₂O at 0° C. The aqueous layer was extracted with EtOAc (3×50 mL), and the combined organic layers were dried over Na₂SO₄, filtered, and concentrated under reduced pressure. The residue was purified by prep-TLC (17% EtOAc/pet. ether) to afford the desired product (250 mg, 67% yield). LCMS (ESI) m/z [M+H] calcd for C₁₂H₂₁NO₃S: 260.13; found: 260.1.

### Step 3: Synthesis of (2S,3S)-1-(tert-butylsulfinyl)-3-cyclopropylaziridine-2-carboxylic acid

A solution of ethyl (2S,3S)-1-(tert-butylsulfinyl)-3-cyclopropylaziridine-2-carboxylate (500.0 mg, 1.928 mmol) in THE (2.0 mL) and H₂O (2.0 mL) at 0° C. was added LiOH⋅H₂O (121.34 mg, 2.89 mmol). The reaction mixture was stirred for 1 h and was then acidified to pH 6 with 1M HCl (aq.). The resulting mixture was extracted with EtOAc (2×10 mL) and the combined organic layers were washed with brine (10 mL), dried over Na₂SO₄, filtered, and the filtrate was concentrated under reduced pressure to afford the desired product (400 mg, 90% yield). LCMS (ESI) m/z [M+H] calcd for C₁₀H₁₇NO₃S: 232.10; found: 232.0.

### Intermediate B-2. Synthesis of (2R,3R)-1-((R)-tert-butylsulfinyl)-3-cyclopropylaziridine-2-carboxylic acid

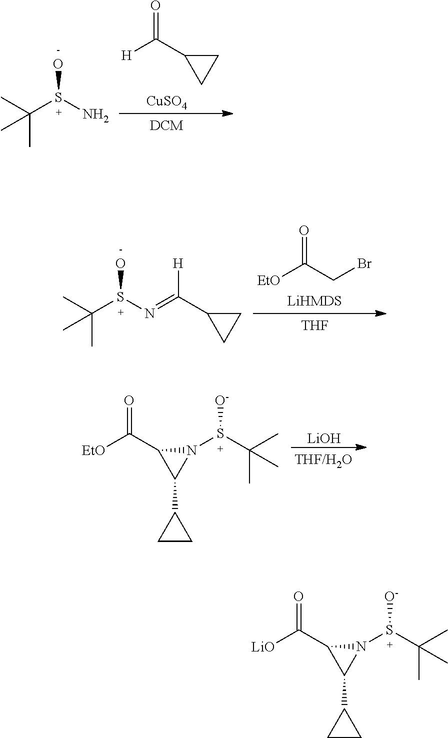

### Step 1: Synthesis of (R,E)-N-(cyclopropylmethylene)-2-methylpropane-2-sulfinamide

To a solution of (R)-2-methylpropane-2-sulfinamide (1.0 g, 8.25 mmol) and cyclopropanecarbaldehyde (1.16 g, 16.55 mmol) in DCM (50 mL) at room temperature was added CuSO₄ (3.95 g, 24.75 mmol). The resulting mixture was stirred overnight. The reaction mixture was then filtered, the filter cake washed with EtOAc, and the filtrate was concentrated under reduced pressure. The residue was purified by prep-TLC (17% EtOAc/pet. ether) to afford the desired product (1.4 g, 98% yield). LCMS (ESI) m/z: [M+H] calcd for C₈H₁₅NOS: 174.10; found 174.1.

### Step 2: Synthesis of ethyl (2R,3R)-1-((R)-tert-butylsulfinyl)-3-cyclopropylaziridine-2-carboxylate

To a solution of 1M LiHMDS (23 mL, 23 mmol) in THE (50.0 mL) at −78° C. was added ethyl bromoacetate (3.83 g, 22.95 mmol). The resulting mixture was warmed to −70° C. and stirred for 1 h. To the reaction mixture was then added (R,E)-N-(cyclopropylmethylene)-2-methylpropane-2-sulfinamide (2.0 g, 11.48 mmol). The resulting mixture was stirred for 1 h at −70° C. The reaction mixture was warmed to 0° C. and quenched with H₂O. The aqueous layer was extracted with EtOAc (3×100 mL). The combined organic layers were washed with brine, dried over Na₂SO₄, filtered, and concentrated under reduced pressure. The residue was purified by prep-TLC (25% EtOAc/pet. ether) to afford the desired product (1.8 g, 61% yield). LCMS (ESI) m/z: [M+H] calcd for C₁₂H₂₁NO₃S: 306.14; found 260.13.

### Step 3: Synthesis of (2R,3R)-1-((R)-tert-butylsulfinyl)-3-cyclopropylaziridine-2-carboxylic acid

To a solution of ethyl (2R,3R)-1-((R)-tert-butylsulfinyl)-3-cyclopropylaziridine-2-carboxylate (900.0 mg, 3.47 mmol) in THE (3.0 mL) and H₂O (3.0 mL) at 0° C. was added LiOH⋅H₂O (218.4 mg,

5.21 mmol). The resulting mixture was stirred for 1 h and was then quenched by H₂O. The aqueous layer was extracted with EtOAc (3×50) and the combined organic layers were washed with brine, dried over Na₂SO₄, filtered, and concentrated under reduced pressure to afford the desired crude product (400 mg, 30% yield). LCMS (ESI) m/z: [M+H] calcd for C₁₀H₁₇NO₃S: 232.10; found 232.1.

### Intermediate B-3: Synthesis of Ethyl (2R,3R)-3-cyclopropylaziridine-2-carboxylate

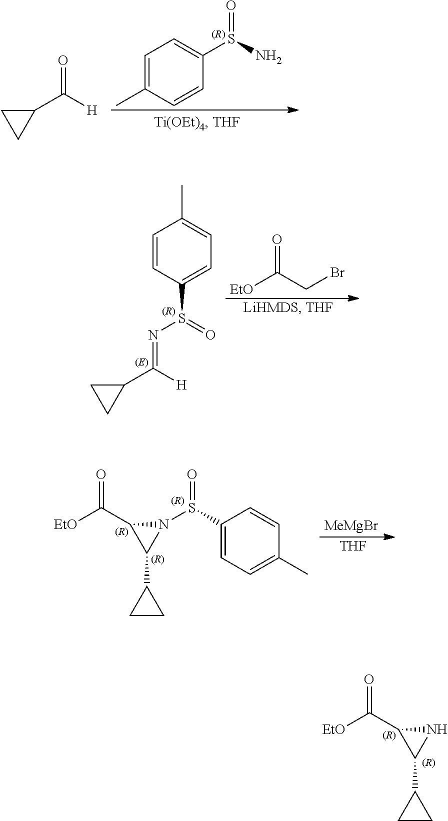

### Step 1: Synthesis of (R,E)-N-(cyclopropylmethylene)-4-methylbenzenesulfinamide

To a solution of cyclopropanecarbaldehyde (6 g, 85.60 mmol) in THE (120 mL) was added (R)-4-methylbenzenesulfinamide (13.29 g, 85.60 mmol) and Ti(OEt)₄ (39.05 g, 171.21 mmol) at room temperature under N₂. The mixture was stirred at 75° C. for 2 h. The reaction mixture was poured into brine/H₂O (1:1, 600 mL) at 0-15° C. The mixture was filtered through a pad of Celite and the pad was washed with EtOAc (6×200 mL). The combined filtrates were extracted with EtOAc (2×200 mL). The combined organic layers were washed with brine (200 mL), dried over Na₂SO₄, filtered and concentrated under reduced pressure to give a residue. The crude product was purified by silica gel column chromatography. (0→10% EtOAc/pet. ether) to give the product (14.6 g, 82% yield) as a solid.

### Step 2: Synthesis of ethyl (2R,3R)-3-cyclopropyl-1-((R)-p-tolylsulfinyl)aziridine-2-carboxylate

To a solution of ethyl 2-bromoacetate (23.52 g, 140.86 mmol) in THE (700 mL) was added LiHMDS (1 M, 140.86 mL) at −70° C. over 10 min under N₂. The mixture was stirred at −70° C. for 20 min. A solution of (R,E)-N-(cyclopropylmethylene)-4-methylbenzenesulfinamide (14.6 g, 70.43 mmol) in THE (150 mL) was added into the reaction solution at −70° C. for 10 min. Then the mixture was stirred at −70° C. for 1 h 20 min under N₂. The reaction mixture was poured into cold H₂O (1.2 L) and stirred at room temperature for 5 min. The aqueous layer was extracted with EtOAc (3×300 mL). The combined organic layers were washed with brine (300 mL), dried over Na₂SO₄, filtered and concentrated under reduced pressure to give a residue. The crude product was purified by silica gel column chromatography. (0→10% EtOAc/pet. ether) to give the product (11 g, 53% yield) as an oil. LCMS (ESI) m/z [M+H] calcd for C₁₅H₂₀NO₃S: 294.11; found: 294.1.

### Step 3: Synthesis of ethyl (2R,3R)-3-cyclopropylaziridine-2-carboxylate

Ethyl (2R,3R)-3-cyclopropyl-1-[(R)-p-tolylsulfinyl]aziridine-2-carboxylate (6 g, 20.45 mmol) was dissolved in anhydrous THE (300 mL). MeMgBr (3 M, 13.63 mL) was added dropwise at −65° C. over 40 min under N₂. The reaction mixture was stirred for 5 min. Sat. aq. NH₄Cl (90 mL) was added dropwise at −65° C. The cooling bath was removed, and the reaction mixture was warmed to room temperature. EtOAc (300 mL) was added and the organic layer was separated and concentrated under reduced pressure. The residue was purified by silica gel column chromatography (0→50% EtOAC/pet. ether) to afford the product as an oil.

### Intermediate B-4: Synthesis of Lithium (2R,3R)-3-cyclopropyl-1-((R)-p-tolylsulfinyl)aziridine-2-carboxylate

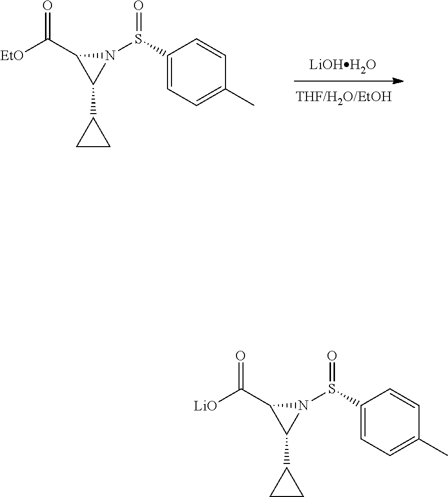

To a solution of ethyl (2R,3R)-3-cyclopropyl-1-((R)-p-tolylsulfinyl)aziridine-2-carboxylate (380 mg, 1.30 mmol) in THE (1.6 mL), H₂O (1.2 mL) and EtOH (1.2 mL) was added LiOH⋅H₂O (163.06 mg, 3.89 mmol) at 0° C., then the mixture was stirred at room temperature for 1 h. H₂O (5 mL) was added and the reaction mixture was lyophilized directly to give the product (430 mg, crude) as a solid, which was used directly in the next step. LCMS (ESI) m/z [M+H] calcd for C₁₃H₁₆NO₃S:266.08; found: 266.1 Intermediate B-5: Synthesis of (2R,3R)-3-cyclopropyl-1-methylaziridine-2-carboxylic acid

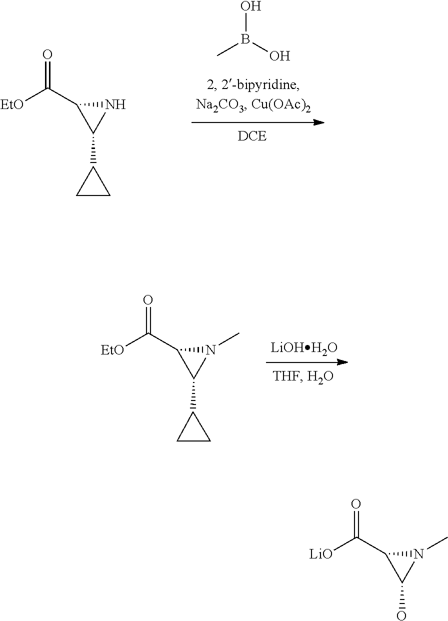

### Step 1: ethyl (2R,3R)-3-cyclopropyl-1-methylaziridine-2-carboxylate

To a solution of ethyl (2R,3R)-3-cyclopropylaziridine-2-carboxylate (400 mg, 2.58 mmol) in DCE (8 mL) was added methylboronic acid (462.85 mg, 7.73 mmol), 2,2′-bipyridine (402.54 mg, 2.58 mmol), Cu(OAc)₂ (468.14 mg, 2.58 mmol), and Na₂CO₃ (819.54 mg, 7.73 mmol). The reaction mixture was stirred at 45° C. for 40 h. The mixture was poured into aq. NH₄Cl (15 mL) and extracted with DCM (3×15 mL), the combined organic phases were washed with brine (20 mL), dried with anhydrous Na₂SO₄, filtered and concentrated under reduced pressure. The residue was purified by silica gel column chromatography (0→50% EtOAc/pet. ether) to give the product (230 mg, 53% yield) as an oil. LCMS (ESI) m/z [M+H] calcd for C₉H₁₆NO₂: 170.1; found: 170.1.

### Step 2: (2R,3R)-3-cyclopropyl-1-methylaziridine-2-carboxylic acid

To a solution of ethyl (2R,3R)-3-cyclopropyl-1-methylaziridine-2-carboxylate (230 mg, 1.36 mmol) in THE (2 mL) was added a solution of LiOH⋅H₂O (114.07 mg, 2.72 mmol) in H₂O (1 mL). The reaction mixture was stirred at room temperature for 1 h. The pH was adjusted to about 8 with 0.5N HCl at 0° C., and the solution was lyophilized directly to give the product (230 mg, crude) as a solid.

### Intermediate B-6: Synthesis of (S)-1-((benzyloxy)carbonyl)-2-methylaziridine-2-carboxylic acid

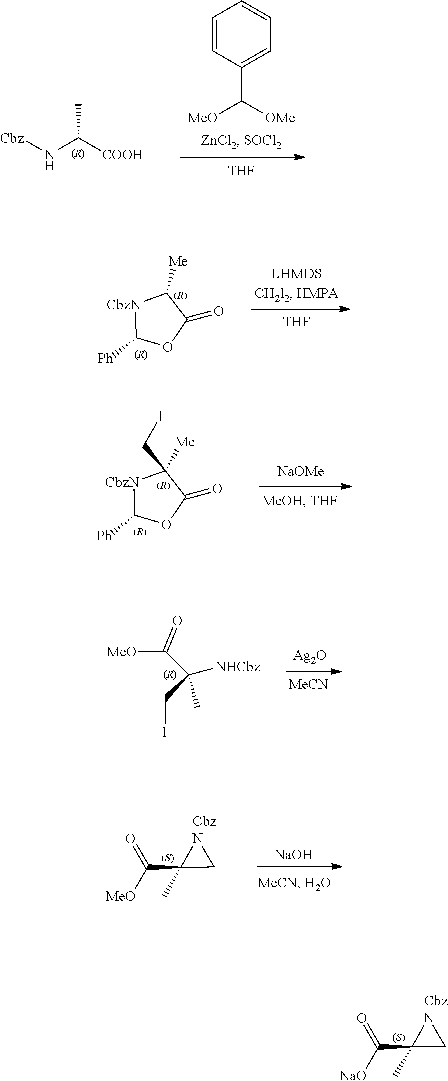

### Step 1: Synthesis of benzyl (2R,4R)-4-methyl-5-oxo-2-phenyloxazolidine-3-carboxylate

To a mixture of ((benzyloxy)carbonyl)-D-alanine (5 g, 22.40 mmol) and (dimethoxymethyl)benzene (3.75 g, 24.64 mmol) in THE (35 mL) was added SOCl₂ (2.93 g, 24.64 mmol) in one portion at 0° C. After the mixture was stirred for 10 min, ZnCl₂ (3.36 g, 24.64 mmol) was added to the solution. Then the mixture was stirred at 0° C. for 4 h. The reaction mixture was quenched by dropwise addition of cold H₂O and adjusted to pH 5 with sat. aq. NaHCO₃, then extracted with EtOAc (3×50 mL). The organic layer was washed with a sat. aq. NaHCO₃ (30 mL) and brine (30 mL), dried over Na₂SO₄, and concentrated. The residue was purified by silica gel column chromatography (0→20% EtOAc/pet. ether) to afford the product (3.19 g, 46% yield) as an oil. LCMS (ESI) m/z [M+H] calcd for C₁₈H₁₈NO₄: 311.14; found: 312.1.

### Step 2: Synthesis of benzyl (2R,4R)-4-(iodomethyl)-4-methyl-5-oxo-2-phenyloxazolidine-3-carboxylate

To a mixture of THE (50 mL), HMPA (8.50 g, 47.44 mmol) was added LiHMDS (1 M, 10.55 mL) under N₂ at room temperature. This solution was cooled to −70° C. and a solution of benzyl (2R,4R)-4-methyl-5-oxo-2-phenyl-oxazolidine-3-carboxylate (3.19 g, 10.25 mmol) in THE (14 mL) was added dropwise. After stirring an additional 30 min, a solution of diiodomethane (8.23 g, 30.74 mmol) in THE (14 mL) was added dropwise. The mixture was stirred at −70° C. for 90 min. sat. aq. NH₄Cl (50 mL) was added to the reaction mixture at 0° C. and extracted with EtOAc (3×30 mL). The combined organic layers were washed with brine (80 mL), dried over Na₂SO₄, filtered and concentrated under reduced pressure. The residue was purified by silica gel column chromatography (0→20% EtOAc/pet. ether) to afford the product (3.03 g, 66% yield) as an oil. LCMS (ESI) m/z [M+H] calcd for C₁₉H₁₉INO₄: 451.26; found: 452.0.

### Step 3: Synthesis of methyl (R)-2-(((benzyloxy)carbonyl)amino)-3-iodo-2-methylpropanoate

To a solution of benzyl (2R,4R)-4-(iodomethyl)-4-methyl-5-oxo-2-phenyl-oxazolidine-3-carboxylate (3 g, 6.65 mmol) in THE (50 mL) was added NaOMe (2.39 g, 13.30 mmol) in MeOH (22.5 mL) dropwise over 10 min at −40° C. under N₂. The mixture was stirred at −40° C. for 2 h, then warmed to room temperature and stirred for 30 min. The reaction was quenched by the addition of H₂O (50 mL), and the resulting mixture was extracted with EtOAc (3×30 mL). The combined organic layers were washed with brine (80 mL), dried over Na₂SO₄, filtered, and concentrated under reduced pressure. The residue was purified by silica gel column chromatography (0→20% EtOAc/pet. ether) to afford the product (2.04 g, 81% yield) as an oil. LCMS (ESI) m/z [M+H] calcd for C₁₃H₁₇INO₄: 377.18; found: 378.0.

### Step 4: Synthesis of 1-benzyl 2-methyl (S)-2-methylaziridine-1, 2-dicarboxylate

To a solution of (R)-2-(((benzyloxy)carbonyl)amino)-3-iodo-2-methylpropanoate (1 g, 2.65 mmol) in MeCN (100 mL) was added Ag₂O (1.84 g, 7.95 mmol) at room temperature. The mixture was heated at 90° C. for 30 min. After the reaction was cooled to room temperature the mixture was filtered through Celite and the filtrate concentrated under reduced pressure. This residue was extracted with EtOAc (100 mL), and the organic layer was filtered through Celite and concentrated under reduced pressure to afford the product (630 mg, crude) as an oil. LCMS (ESI) m/z [M+H] calcd for C₁₃H₁₆NO₄: 249.27; found: 250.1.

### Step 5: Synthesis of (S)-1-((benzyloxy)carbonyl)-2-methylaziridine-2-carboxylic acid

To a solution of 1-benzyl 2-methyl (S)-2-methylaziridine-1,2-dicarboxylate (630 mg, 2.53 mmol) in MeCN (3.2 mL) was add a solution of NaOH (151.65 mg, 3.79 mmol) in H₂O (3.2 mL) at 0° C. The mixture was stirred at 0° C. for 30 min. The reaction mixture was diluted with H₂O (10 mL) and lyophilized to give the product (652.65 mg, crude) as solid.

### Intermediate B-7: Synthesis of (R)-1-((benzyloxy)carbonyl)-2-methylaziridine-2-carboxylic acid

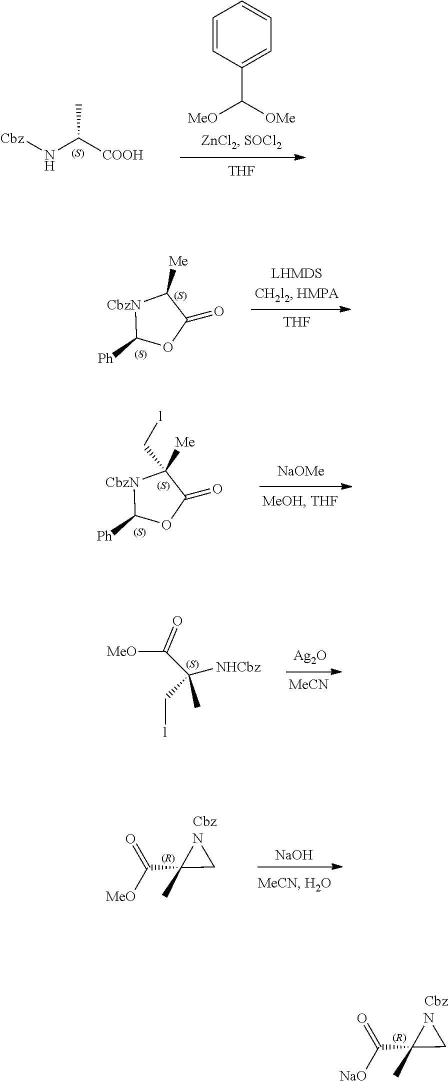

### Step 1: Synthesis of benzyl (2S,4S)-4-methyl-5-oxo-2-phenyloxazolidine-3-carboxylate

To a solution of ((benzyloxy)carbonyl)-L-alanine (5 g, 22.40 mmol) and (dimethoxymethyl)benzene (3.51 g, 23.07 mmol) in THE (36 mL) was added SOCl₂ (2.93 g, 24.64 mmol) in one portion at 0° C. The mixture was stirred for 10 min and then ZnCl₂ (1.15 mL, 24.64 mmol) was added. The mixture was then stirred at 0° C. for 4 h. The reaction mixture was quenched by the dropwise addition of cold H₂O, adjusted to pH 5 with sat. NaHCO₃, then extracted with EtOAc (2×30 mL). The organic phase was washed with a sat. aq. NaHCO₃ (30 mL) and brine (30 mL), dried with anhydrous Na₂SO₄, and concentrated under reduced pressure. The residue was purified by silica gel column chromatography (0→20% EtOAc/pet. ether) to afford the product (3.7 g, 53% yield) as an oil.

### Step 2: Synthesis of benzyl (2S,4S)-4-(iodomethyl)-4-methyl-5-oxo-2-phenyloxazolidine-3-carboxylate

A mixture of HMPA (9.33 g, 52.05 mmol) and LiHMDS (1 M, 11.58 mL) in THE (52 mL) was stirred at room temperature under N₂, and was then cooled to −70° C. A solution of benzyl (2S,4S)-4-methyl-5-oxo-2-phenyloxazolidine-3-carboxylate (3.5 g, 11.24 mmol) in THE (15 mL) was added dropwise. After stirring for 30 min, a solution of diiodomethane (9.03 g, 33.73 mmol) in THE (7 mL) was added dropwise. The mixture was stirred at −70° C. for 90 min, then sat. NH₄Cl (50 mL) was added to the reaction mixture at 0° C. and the solution was extracted with EtOAc (3×30 mL). The combined organic layers were washed with brine (80 mL), dried over anhydrous Na₂SO₄, filtered and concentrated under reduced pressure. The residue was purified by column chromatography (0→20% EtOAc/pet. ether) to give the product (2.39 g, 47% yield) as an oil.

### Step 3: Synthesis of methyl (S)-2-(((benzyloxy)carbonyl)amino)-3-iodo-2-methylpropanoate

To a solution of benzyl (2S,4S)-4-(iodomethyl)-4-methyl-5-oxo-2-phenyloxazolidine-3-carboxylate (2.39 g, 5.30 mmol) in THE (40 mL) was added NaOMe (1.91 g, 10.59 mmol) in MeOH (19 mL) dropwise over 10 min at −40° C. under N₂. The mixture was stirred at −40° C. for 2 h, then warmed to −20° C. and stirred for 0.5 h. The reaction was quenched by addition of H₂O (50 mL), and the resulting mixture was extracted with EtOAc (3×30 mL). The combined organic layers were washed with brine (80 mL), dried over Na₂SO₄, filtered, and concentrated under reduced pressure. The residue was purified by column chromatography (0→20% EtOAc/pet. ether) to give the product (1.37 g, 69% yield) as an oil.

### Step 4: Synthesis of 1-benzyl 2-methyl (R)-2-methylaziridine-1,2-dicarboxylate

To a mixture of methyl (2S)-2-(benzyloxycarbonylamino)-3-iodo-2-methyl-propanoate (1.37 g, 3.63 mmol) in MeCN (14 mL) was added Ag₂O (2.53 g, 10.90 mmol) in one portion at room temperature. The mixture was stirred at 90° C. for 30 min, then the mixture was vacuum filtered through Celite and the filtrate was concentrated under reduced pressure to give the product (790 mg, 87% yield) as an oil.

LCMS: (ESI) m/z [M+H] calcd for C₁₃H₁₆NO₄:250.10; found 250.1.

### Step 5: Synthesis of (R)-1-((benzyloxy)carbonyl)-2-methylaziridine-2-carboxylic acid

To a mixture of 1-benzyl 2-methyl (R)-2-methylaziridine-1,2-dicarboxylate (790 mg, 3.17 mmol) in MeCN (7.6 mL) and H₂O (7.6 mL) was added NaOH (126.77 mg, 3.17 mmol) in one portion at 0° C. under N₂. The mixture was stirred at 0° C. for 0.5 h. The reaction was added H₂O (8 mL) and lyophilized to give (R)-1-((benzyloxy)carbonyl)-2-methylaziridine-2-carboxylic acid (850 mg, crude, Na) as white solid.

### Intermediate B-8: Synthesis of (2R,3R)-3-methyloxirane-2-carboxylic acid

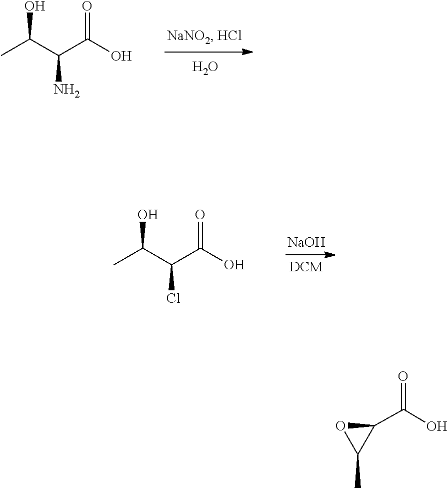

### Step 1: Synthesis of (2S,3R)-2-chloro-3-hydroxybutanoic acid

To a mixture of (2S,3R)-2-amino-3-hydroxy-butanoic acid (25 g, 209.87 mmol) in HCl (148.50 g, 1.55 mol) and H₂O was added NaNO₂ (54.30 g, 314.81 mmol) at 0° C. The reaction mixture was stirred at 0° C. for 3 h, and was then extracted with EtOAc (3×200 mL). The combined organic layer was washed with brine (100 mL), dried over Na₂SO₄, filtered and the filtrate was concentrated under reduced pressure to give the product (27 g, crude) as an oil, which was used directly to the next step. LCMS (ESI) m/z [M−H] calcd for C₄H₆ClO₃: 137.01; found: 137.0.

### Step 2: Synthesis of (2R,3R)-3-methyloxirane-2-carboxylic acid

To a mixture of (2S,3R)-2-chloro-3-hydroxy-butanoic acid (27 g, 194.88 mmol) in DCM (300 mL) was added NaOH (48.99 g, 428.73 mmol) at 10° C. The mixture was stirred at 10° C. for 3 h. The aqueous layer was extracted with DCM (2×50 mL). The pH of aqueous layer was adjusted to about 1-2 by adding 25% HCl and was then extracted with EtOAc (4×100 mL). The organic layer was washed with brine (30 mL), dried over Na₂SO₄, filtered and the filtrate was concentrated under reduced pressure to give the product (6 g, 30% yield) as an oil, which was used directly to the next step. LCMS (ESI) m/z [M−H] calcd for C₄H₅O₃: 101.03; found: 101.0

### Intermediate B-9: Synthesis of Lithium (2R,3R)-3-cyclopropyloxirane-2-carboxylate

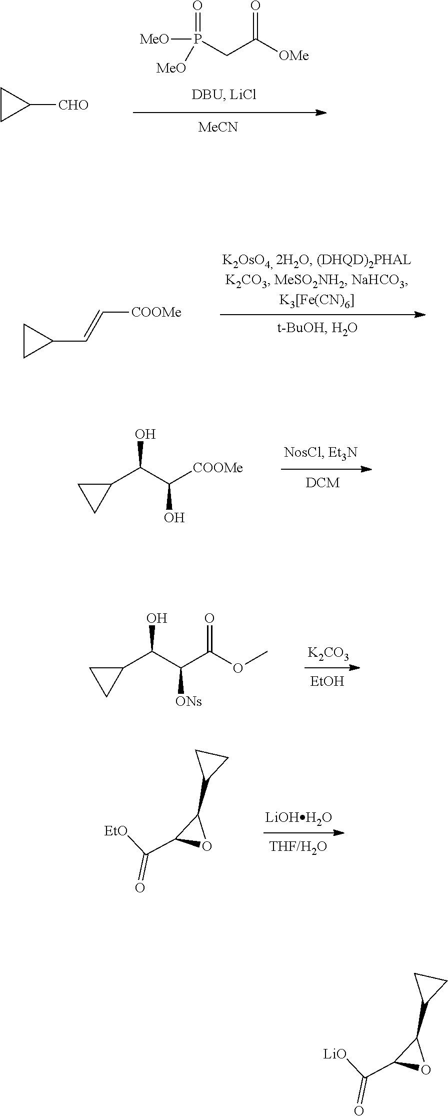

### Step 1: Synthesis of methyl (E)-3-cyclopropylacrylate

To a mixture of methyl 2-(dimethoxyphosphoryl)acetate (31.18 g, 171.21 mmol) in MeCN (100 mL) was added DBU (26.06 g, 171.21 mmol) and LiCl (9.07 g, 214.01 mmol) at 0° C. followed by cyclopropanecarbaldehyde (10 g, 142.67 mmol). The mixture was stirred at room temperature for 12 h then quenched with H₂O (300 mL) and extracted with EtOAc (2×150 mL). The combined organic layers were washed with brine (100 mL), dried over Na₂SO₄, filtered and the filtrate was concentrated under reduced pressure to give a residue. The residue was purified by silica gel column chromatography (0→20% EtOAc/pet. ether) to give the product (9 g, 50% yield) as an oil.

### Step 2: Synthesis of methyl (2S,3R)-3-cyclopropyl-2,3-dihydroxypropanoate

A mixture of K₃[Fe(CN)₆] (27.40 g, 83.23 mmol), K₂CO₃ (11.50 g, 83.23 mmol), MeSO2NH₂ (2.64 g, 27.74 mmol), NaHCO₃(6.99 g, 83.23 mmol) in t-BuOH (210 mL) and H₂O (140 mL) was stirred at room temperature for 10 min. K₂OsO₄·2H₂O (40.89 mg, 110.98 μmol) and (DHQD)₂PHAL (216.12 mg, 277.44 μmol) were then added. The mixture was stirred at for 30 min, then cooled to 0° C. Methyl (E)-3-cyclopropylacrylate (3.5 g, 27.74 mmol, 1 eq) in t-BuOH (70 mL) was added to the mixture and stirred at room temperature for 15 h. The mixture was quenched with sat. Na₂S₂O₃ (100 mL) and was then extracted with EtOAc (3×200 mL). The combined organic layers were washed with brine (100 mL) and dried over Na₂SO₄, filtered and the filtrate was concentrated under reduced pressure to give a residue. The residue was purified by silica gel column chromatography (0→50% EtOAc/pet. ether) to afford the product (4.2 g, 47% yield) as a solid.

### Step 3: Synthesis of methyl (2S,3R)-3-cyclopropyl-3-hydroxy-2-(((4-nitrophenyl)sulfonyl) oxy)propanoate

To a mixture of methyl (2S,3R)-3-cyclopropyl-2,3-dihydroxypropanoate (4 g, 24.97 mmol) and Et₃N (3.79 g, 37.46 mmol) in DCM (40 mL) was added dropwise 4-nitrobenzenesulfonyl chloride (6.09 g, 27.47 mmol) in DCM (10 mL) at 0° C. and stirred at room temperature for 12 h. The mixture was poured into H₂O (50 mL) and extracted with DCM (2×50 mL). The combined organic layers were washed with brine (30 mL), dried over Na₂SO₄, filtered and the filtrate was concentrated under reduced pressure to give a residue. The residue was purified by silica gel column chromatography (0→100% EtOAc/pet. ether) to give the product (5.82 g, 68% yield) as an oil.

### Step 4: Synthesis of ethyl (2R,3R)-3-cyclopropyloxirane-2-carboxylate

To a mixture of methyl (2S,3R)-3-cyclopropyl-3-hydroxy-2-(((4-nitrophenyl)sulfonyl)oxy) propanoate (1 g, 2.90 mmol) in EtOH (20 mL) was added K₂CO₃ (800.44 mg, 5.79 mmol). The mixture was stirred at 15° C. for 12 h. The mixture was poured into sat. NaHCO₃ (50 mL) and extracted with DCM (3×40 mL). The combined organic layers were washed with brine (30 mL), dried over Na₂SO₄, filtered and the filtrate was concentrated under reduced pressure to give a residue. The residue was purified by silica gel column chromatography (0→30% EtOAc/pet. ether) to give the product (0.3 g, crude) as an oil, which was used directly in the next step.

### Step 5: Synthesis of lithium (2R,3R)-3-cyclopropyloxirane-2-carboxylate

To a solution of ethyl (2R,3R)-3-cyclopropyloxirane-2-carboxylate (300 mg, 1.92 mmol) in THE (3 mL) was added LiOH⋅H₂O (161.20 mg, 3.84 mmol) in H₂O (1.5 mL). The mixture was stirred at 0° C. for 1 h. H₂O (20 mL) was added and the mixture was lyophilized directly to give the product (200 mg, crude) as solid. LCMS (ESI) m/z [M−H] calcd for C₆H₇O₂: 127.0; found: 127.0.

### Synthesis of (2S)-2-cyclopentyl-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-7-((R)-oxirane-2-carbonyl)-2,7-diazaspiro[4.4]nonan-2-yl)acetamide

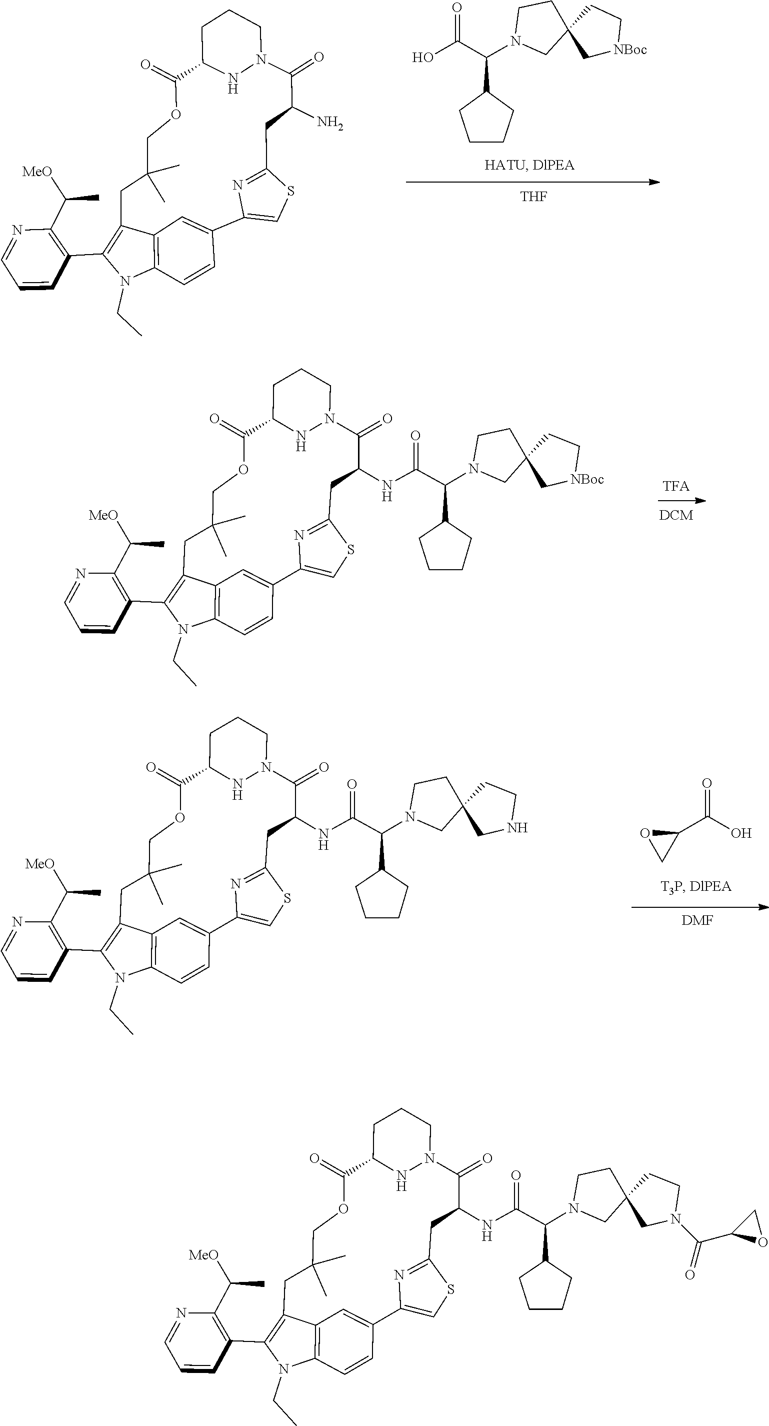

### Step 1: Synthesis of tert-butyl (5S)-7-((1S)-1-cyclopentyl-2-(((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.4]nonane-2-carboxylate

To a solution of (S)-2-((S)-7-(tert-butoxycarbonyl)-2,7-diazaspiro[4.4]nonan-2-yl)-2-cyclopentylacetic acid (239.71 mg, 680.09 μmol) in THE (6.6 mL) was added HATU (278.48 mg, 732.41 μmol) and DIPEA (202.84 mg, 1.57 mmol) at 0° C. The mixture was stirred at room temperature for 30 min and then (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (330 mg, 523.15 μmol) was added. The mixture was stirred at room temperature for 1 h. The reaction mixture was poured into cold H₂O (30 mL) and the aqueous phase was extracted with EtOAc (3×10 mL). The combined organic phases were washed with brine (10 mL), dried with anhydrous Na₂SO₄, filtered and concentrated under reduced pressure to afford the product as solid, which was used directly in the next step. LCMS (ESI) m/z [M+H] calcd for C₅₃H₇₃N₈O₇S: 965.5; found: 965.6

### Step 2: Synthesis of (2S)-2-cyclopentyl-N-((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)-2-((S)-2,7-diazaspiro[4.4]nonan-2-yl)acetamide

To a solution of tert-butyl (5S)-7-((1 S)-1-cyclopentyl-2-(((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.4]nonane-2-carboxylate (700 mg, 725.20 μmol) in DCM (7 mL) was added TFA (1.65 g, 14.50 mmol) at 0° C., then the reaction was stirred at room temperature for 1 h. The reaction mixture was added dropwise to sat. aq. NaHCO₃ (50 mL) at 0° C. The mixture was extracted with DCM (3×30 mL) and the combined organic phases were washed with brine (20 mL), dried with anhydrous Na₂SO₄, filtered and concentrated under reduced pressure to give the product (400 mg, crude) as solid, which was used directly in the next step. LCMS (ESI) m/z [M+H] calcd for C₄₈H₆₅N₈₀₅S: 865.5; found: 865.5.

### Step 3: Synthesis of (2S)-2-cyclopentyl-N-((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)-2-((S)-7-((R)-oxirane-2-carbonyl)-2,7-diazaspiro[4.4]nonan-2-yl)acetamide

To a solution of (2S)-2-cyclopentyl-N-((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)-2-((S)-2,7-diazaspiro[4.4]nonan-2-yl)acetamide (400 mg, 416.12 μmol) and (R)-oxirane-2-carboxylic acid (105.83 mg, 832.24 μmol, K) in DMF (4 mL) was added DIPEA (376.46 mg, 2.91 mmol) and T₃P (317.76 mg, 499.34 μmol) at 0° C., then the reaction was stirred at room temperature for 1 h. The reaction was poured into cold H₂O (50 mL). and the aqueous phase was extracted with EtOAc (3×30 mL). The combined organic phases were washed with brine (2×20 mL), dried with anhydrous Na₂SO₄, filtered and concentrated under reduced pressure and purified by reverse phase chromatography (45→70% MeCN/H₂O, 0.1% NH₄HCO₃) to afford the product (40.83 mg, 10% yield) as solid. LCMS (ESI) m/z [M+H] calcd for C₅₁H₆₇N₈O₇S: 935.49; found: 935.4.

### Synthesis of (2S)-2-cyclopentyl-2-((S)-7-((2R,3R)-3-cyclopropyl-1-methylaziridine-2-carbonyl)-2,7-diazaspiro[4.4]nonan-2-yl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

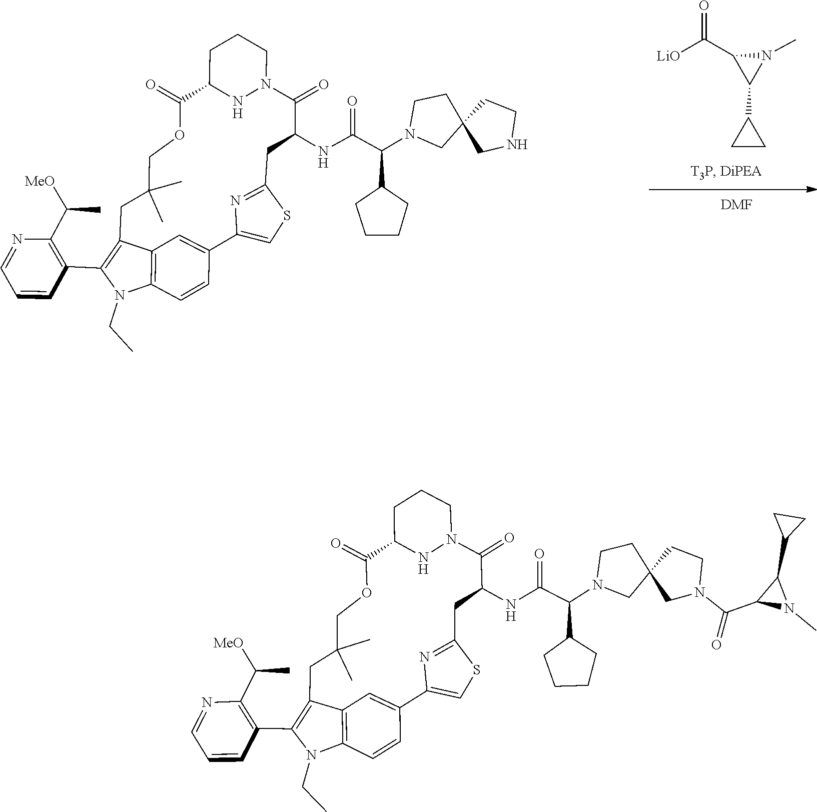

To a solution of (2S)-2-cyclopentyl-N-((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-11H-8-oxa-2(4,2)-thiazola-1(5,3)-indola-6(1,3)-pyridazinacycloundecaphane- 4-yl)-2-((S)-2,7-diazaspiro[4.4]nonan-2-yl)acetamide (250 mg, 288.97 μmol) and (2R,3R)-3-cyclopropyl-1-methylaziridine-2-carboxylic acid (64.20 mg, 346.77 μmol Li) in DMF (2.5 mL) was added DIPEA (261.43 mg, 2.02 mmol), T3P (239.06 mg, 375.66 μmol) at 0° C., then the reaction was stirred at room temperature for 1 h under N₂. The reaction was poured into sat. aq. NH₄Cl (30 mL) and the aqueous phase was extracted with EtOAc (3×10 mL). The combined organic phases were washed with brine (10 mL), dried with anhydrous Na₂SO₄, filtered and concentrated under reduced pressure to give crude product which was purified by reverse phase chromatography (50→80% MeCN/H₂O, 0.1% NH₄HCO₃) to give the product (57.50 mg, 20% yield,) as a solid. LCMS (ESI) m/z [M+H] calcd for C₅₅H₇₄N₉O₆S: 988.55; found: 988.5.

### Synthesis of (2S)-2-cyclopentyl-2-((S)-7-((2R,3R)-1,3-dicyclopropylaziridine-2-carbonyl)-2,7-diazaspiro[4.4]nonan-2-yl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

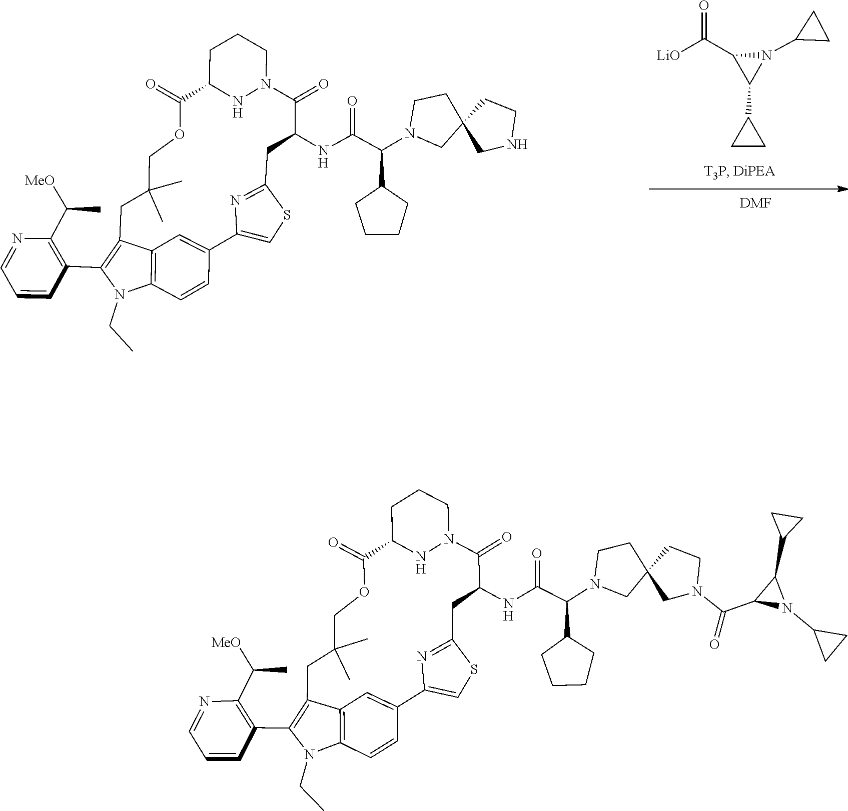

To a solution of (2S)-2-cyclopentyl-N-((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)- pyridazinacycloundecaphane-4-yl)-2-((S)-2,7-diazaspiro[4.4]nonan-2-yl)acetamide (350 mg, 404.56 μmol) in THE (7 mL) was added (2R,3R)-1,3-dicyclopropylaziridine-2-carboxylic acid (84.54 mg, 485.47 μmol, Li), T3P (334.68 mg, 525.93 μmol), and DIPEA (366.01 mg, 2.83 mmol). The reaction mixture was stirred at room temperature for 1 h. The mixture was poured into aq. NH₄Cl (10 mL) and extracted with EtOAc (3×10 mL), and the combined organic phases were washed with brine (15 mL), dried with anhydrous Na₂SO₄, filtered and concentrated under reduced pressure. The residue was purified by reverse phase chromatography (50→85% MeCN/H₂O 0.1% NH₄HCO₃) to give the product (51.7 mg, 12% yield) as a solid. LCMS (ESI) m/z [M+H] calcd for C₅₇H₇₆N₉O₆S: 1014.57; found: 1014.5.

### Synthesis of (2S)-2-((S)-7-((2R,3R)-3-cyclopropylaziridine-2-carbonyl)-2,7-diazaspiro [4.4]nonan-2-yl)-N-((6³S,4S,Z)-1¹-ethyl-12-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methylbutanamide

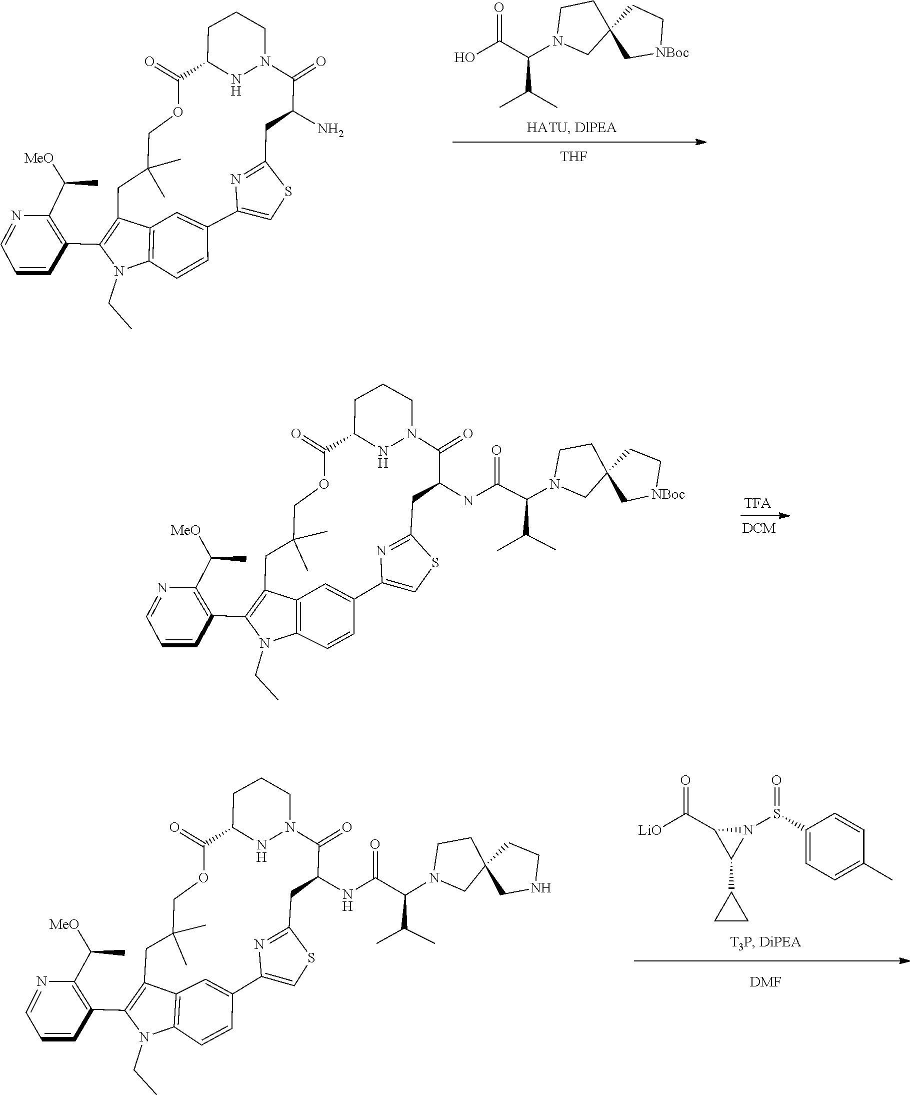

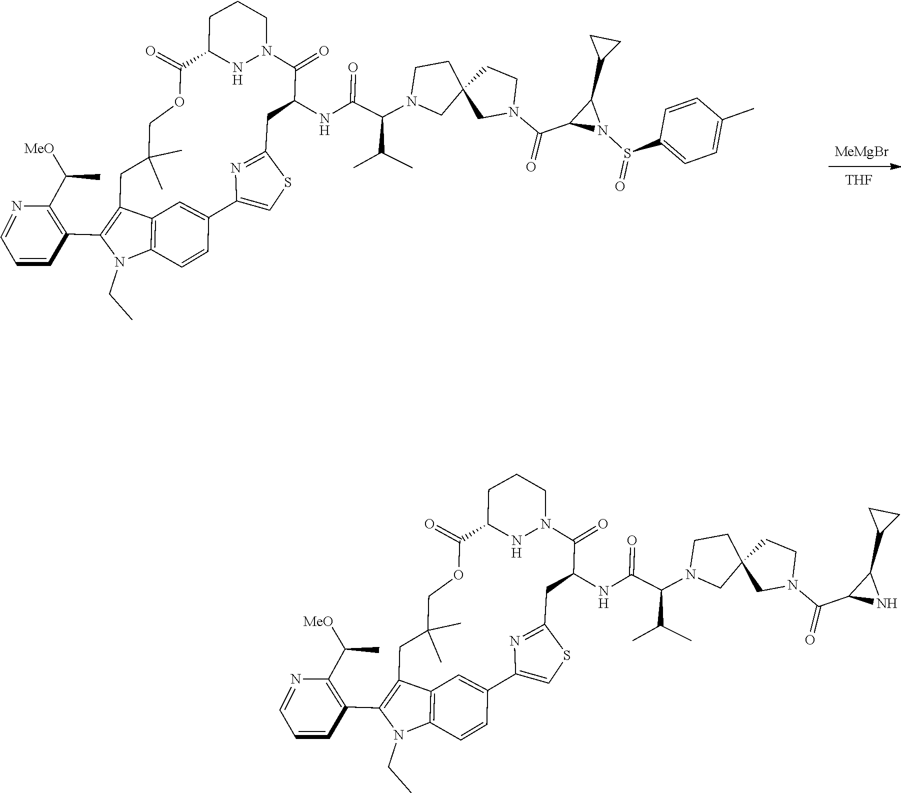

### Step 1: Synthesis of tert-butyl (5S)-7-((2S)-1-(((63S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-2,7-diazaspiro[4.4]nonane-2-carboxylate

To a solution of (S)-2-((S)-7-(tert-butoxycarbonyl)-2,7-diazaspiro[4.4]nonan-2-yl)-3-methylbutanoic acid (6.21 g, 19.02 mmol) in THE (100 mL) was added DIPEA (6.15 g, 47.56 mmol) and HATU (7.53 g, 19.82 mmol) at 0° C., then the mixture was stirred at 0° C. for 20 min, and (6³S,4S,Z)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (10 g, 15.85 mmol) was added. The reaction mixture was stirred at room temperature for 3 h 30 min. The mixture was added to sat. NH₄Cl (500 mL) and the mixture was extracted with EtOAc (3×150 mL). The combined organic phases were washed with brine (2×100 mL), dried with anhydrous Na₂SO₄, filtered and concentrated under reduced pressure. Then the residue was purified by silica gel column chromatography (0→100% EtOAc/pet. ether) to give the product (14.0 g, 80% yield) as a solid.

### Step 2: Synthesis of (2S)—N-((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyl-2-((S)-2,7-diazaspiro[4.4]nonan-2-yl)butanamide

To a solution of tert-butyl (5S)-7-((2S)-1-(((63S,4S,2)-11-ethyl-12-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)-2,7-diazaspiro[4.4]nonane-2-carboxylate (14 g, 12.68 mmol) in DCM (35 mL) was added TFA (43.38 g, 380.47 mmol) at 0° C. The mixture was stirred at room temperature for 30 min. The reaction mixture was then concentrated under reduced pressure and the residue was taken up in DCM (80 mL). The mixture was added dropwise to sat. NaHCO₃ (400 mL) at 0° C. The mixture was extracted with DCM (3×150 mL) and the combined organic phases were washed with brine (2×100 mL), dried with anhydrous Na₂SO₄, filtered and concentrated under reduced pressure to afford the product (10.5 g, 85% yield) as a solid, which was used directly in the next step.

### Step 3: Synthesis of (2S)-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)-3-methylbutanamide

To a solution of (2S)—N-((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyl-2-((S)-2,7-diazaspiro[4.4]nonan-2-yl)butanamide (9.5 g, 11.32 mmol) and (2R,3R)-3-cyclopropyl-1-((R)-p-tolylsulfinyl)aziridine-2-carboxylic acid (4.01 g, 14.72 mmol, Li) in DMF (95 mL) was added DIPEA (8.78 g, 67.93 mmol) and T3P (10.81 g, 16.98 mmol) at 0° C. The mixture was stirred at room temperature for 30 min. The mixture was added to sat. NH₄Cl (500 mL) and the mixture was extracted with EtOAc (3×200 mL). The combined organic phases were washed with brine (3×180 mL), dried with anhydrous Na₂SO₄, filtered and concentrated under reduced pressure. The residue was purified by silica gel column chromatography (0→100% EtOAc/pet. ether) to afford the product (6.89 g, 54% yield) as a solid.

### Step 4: Synthesis of (2S)-2-((S)-7-((2R,3R)-3-cyclopropylaziridine-2-carbonyl)-2,7-diazaspiro [4.4]nonan-2-yl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methylbutanamide

To a solution of (2S)-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methylbutanamide (6.89 g, 6.34 mmol) in THE (68.9 mL) was added MeMgBr (3 M, 16.91 mL) under N₂ at −78° C. The mixture was stirred at −78° C. for 1 h. The reaction mixture was then added into sat. NH₄Cl (50 mL) at 0° C. and then the mixture was extracted with DCM (3×20 mL). The combined organic layers were washed with brine (20 mL), dried with anhydrous Na₂SO₄, filtered and concentrated under reduced pressure. The residue was purified by silica gel column chromatography (0→20% MeOH/EtOAc) to afford the product (4.4 g, 70% yield) as a solid. LCMS (ESI) m/z [M+H] calcd for C₅₂H₇₀N₉O₆S: 948.52; found: 948.6.

### Synthesis of (2S)-2-cyclopentyl-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-7-((R)-2-methylaziridine-2-carbonyl)-2,7-diazaspiro[4.4]nonan-2-yl)acetamide

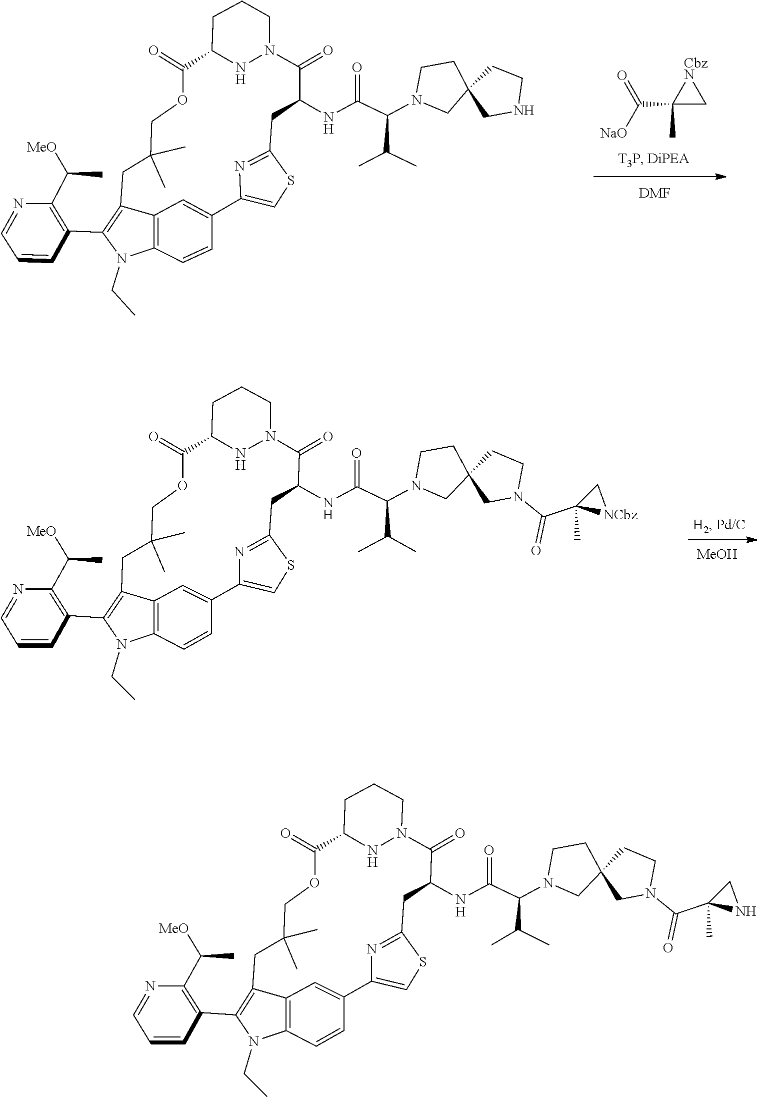

### Step 1: Synthesis of benzyl (2R)-2-((5S)-7-((1S)-1-cyclopentyl-2-(((6³S,4S,2)-1¹-ethyl-12-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)-pyridazinacycloundecaphane-4-yl)amino)-2-oxoethyl)-2,7-diazaspiro[4.4]nonane-2-carbonyl)-2-methylaziridine-1-carboxylate

To a solution of (2S)-2-cyclopentyl-N-((6³S,4S,2)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)- pyridazinacycloundecaphane-4-yl)-2-((S)-2,7-diazaspiro[4.4]nonan-2-yl)acetamide (250 mg, 288.97 μmol) and (R)-1-((benzyloxy)carbonyl)-2-methylaziridine-2-carboxylic acid (81.57 mg, 315.89 μmol, Na) in DMF (2.5 mL) was added DIPEA (186.74 mg, 1.44 mmol) and T3P (183.89 mg, 288.97 μmol) at 0° C., the mixture was stirred at room temperature for 1 h, then sat. NH₄Cl (20 mL) was added at 0° C. The mixture was extracted with EtOAc (3×30 mL) and the combined organic layers were washed with brine (20 mL), dried over Na₂SO₄, filtered and concentrated under reduced pressure to give the crude product. The residue was purified by silica gel column chromatography (0→10% MeOH/EtOAc) to give the product (160 mg, 51% yield) as solid. LCMS (ESI) m/z [M+H] calcd for C₆₀H₇₆N₉O₈S:1082.55; found: 1082.6.

### Step 2: Synthesis of (2S)-2-cyclopentyl-N-((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)-2-((S)-7-((R)-2-methylaziridine-2-carbonyl)-2,7-diazaspiro[4.4]nonan-2-yl)acetamide

To the solution of benzyl (2R)-2-((5S)-7-((1S)-1-cyclopentyl-2-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.4]nonane-2-carbonyl)-2-methylaziridine-1-carboxylate (160 mg, 147.83 μmol) in MeOH (2 mL) was added Pd/C (160 mg). The suspension was degassed under reduced pressure and purged with H₂ several times. The mixture was stirred under H₂ (15 psi) at room temperature for 1 h. The reaction was filtered through Celite and the filtrate was concentrated under reduced pressure. The residue was purified by reverse phase chromatography (40→70% MeCN/H₂O 0.1% NH₄HCO₃) to give the product (56.07 mg, 40% yield) as solid. LCMS: (ESI) m/z [M+H] calcd for C₅₂H₇₀N₉O₆S: 948.52; found: 948.3.

### Synthesis of (2S)-2-cyclopentyl-N-((6³S,4S,Z)-1¹-ethyl-12-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-7-((2R,3R)-3-methyloxirane-2-carbonyl)-2,7-diazaspiro[4.4]nonan-2-yl)acetamide

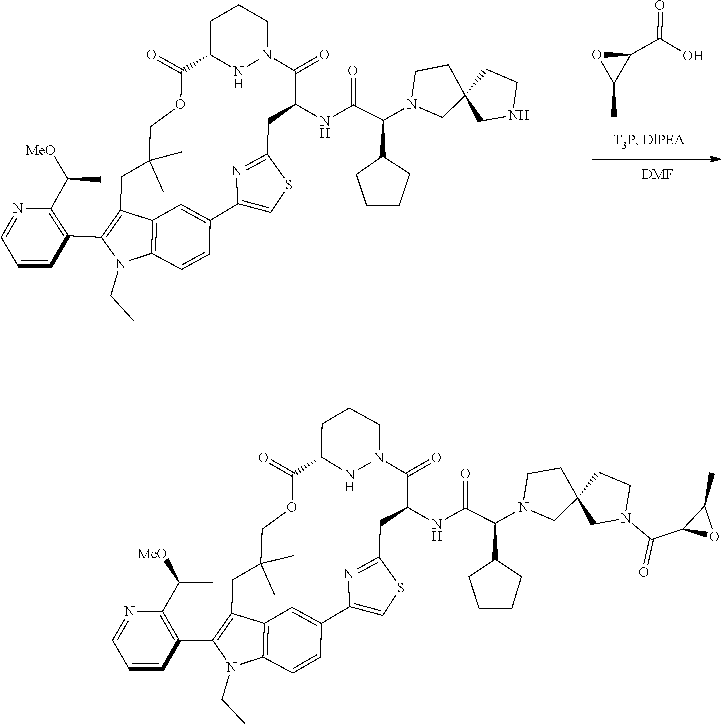

To a solution of (2S)-2-cyclopentyl-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-11H-8-oxa-2(4,2)-thiazola-1(5,3)-indola-6(1,3)-pyridazinacycloundecaphane- 4-yl)-2-((S)-2,7-diazaspiro[4.4]nonan-2-yl) acetamide (0.2 g, 231.18 μmol) and (2R,3R)-3-methyloxirane-2-carboxylic acid (47.20 mg, 462.36 μmol) in DMF (2 mL) was added DIPEA (89.63 mg, 693.53 μmol) and T3P (294.23 mg, 462.36 μmol) at 0° C. The mixture was stirred at room temperature for 2 h, then H₂O (5 mL) was added and the mixture was extracted with EtOAc (3×5 mL). The organic phase was washed with brine (5 mL), dried with anhydrous Na₂SO₄, filtered and concentrated under reduced pressure. The residue was purified by reverse phase chromatography (45→85% MeCN/H₂O 0.1% NH₄HCO₃) to give the product (105 mg, 48% yield) as a solid. LCMS (ESI) m/z [M+H] calcd for C₅₂H₆₉N₈O₇S: 949.50; found: 949.5.

### Synthesis of (2S)-2-cyclopentyl-2-((S)-7-((2R,3R)-3-cyclopropyloxirane-2-carbonyl)-2,7-diazaspiro[4.4]nonan-2-yl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

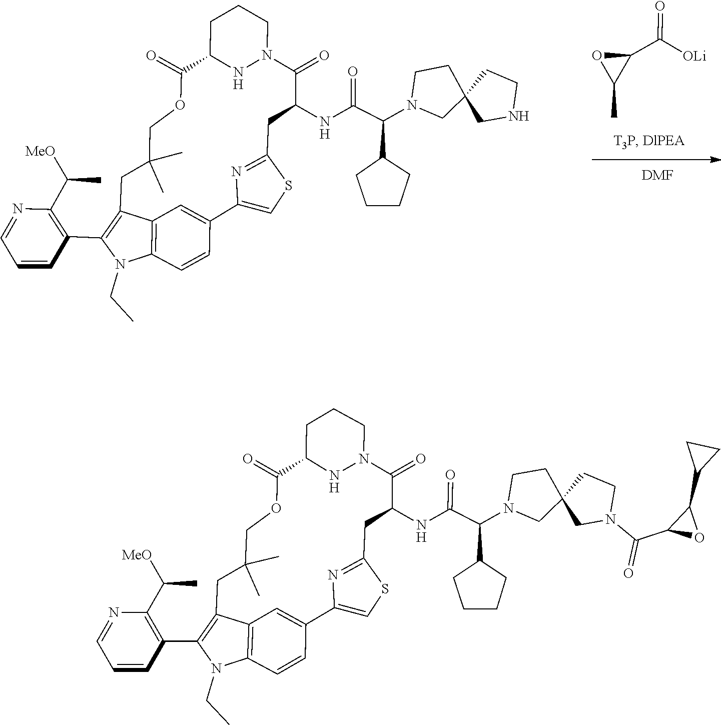

To a solution of lithium (2R,3R)-3-cyclopropyloxirane-2-carboxylate (81.49 mg, 577.94 μmol) and (2S)-2-cyclopentyl-N-((6³S,4S,Z)-1¹-ethyl-1²-(2-((S)-1-methoxyethyl)pyridine-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-2,7-diazaspiro[4.4]nonan-2-yl)acetamide (250 mg, 288.97 μmol) in DMF (3 mL) was added T3P (367.78 mg, 577.94 μmol) and DIPEA (112.04 mg, 866.91 μmol) at 0° C. The mixture was stirred at room temperature for 2 h. The reaction was quenched with H₂O (10 mL), then extracted with EtOAc (3×10 mL). The combined organic layers were washed with brine (10 mL), dried over Na₂SO₄, Filtered and the filtrate was concentrated under reduced pressure to give a residue. The residue was purified by reverse phase chromatography (45→75% MeCN/H₂O 0.1% NH₄HCO₃) to give the product (85.23 mg, 30% yield) as a solid. LCMS (ESI) m/z [M+H] calcd for C₅₄H₇₁N₈O₇S: 975.25; found: 975.5.

### Synthesis of (3S)-1-((2R,3R)-3-cyclopropylaziridine-2-carbonyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide

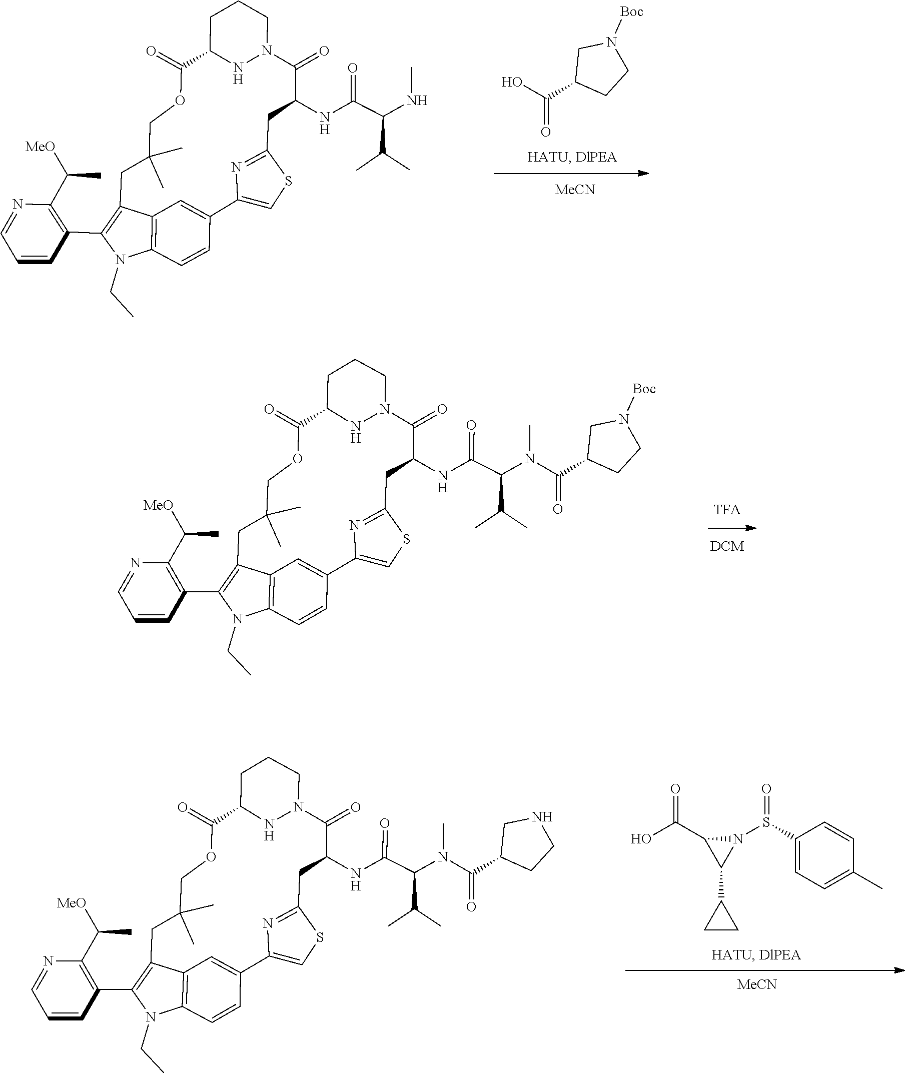

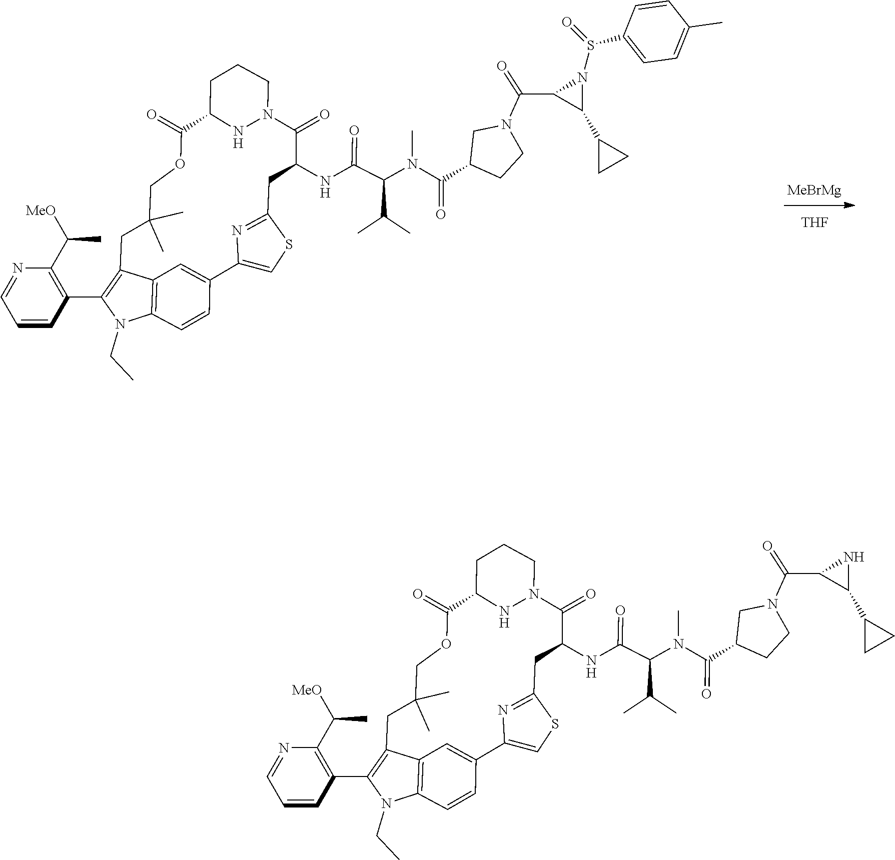

### Step 1: Synthesis of tert-butyl (3S)-3-(((2S)-1-(((63S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)(methyl)carbamoyl)pyrrolidine-1-carboxylate

To a solution of (2S)—N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyl-2-(methylamino)butanamide (0.25 g, 336.04 μmol) in MeCN (7 mL) was added (S)-1-(tert-butoxycarbonyl)pyrrolidine-3-carboxylic acid (216.99 mg, 1.01 mmol), HATU (383.32 mg, 1.01 mmol) and DIPEA (130.29 mg, 1.01 mmol) at 0° C. The mixture was stirred at room temperature for 30 min. The reaction mixture was partitioned between DCM (15 mL) and H₂O (10 mL). The organic phase was separated, washed with brine (2×5 mL), dried over Na₂SO₄, filtered and concentrated under reduced pressure to give a residue. The residue was purified by silica gel column chromatography (50→100% EtOAc/pet. ether) to give the product (0.283 g, 89% yield) as a solid. LCMS (ESI) m/z [M+H] calcd for C₅₀H₆₉N₈O₈S: 941.5; found: 941.4.

### Step 2: Synthesis of (3S)—N-((2S)-1-(((63S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)-N-methylpyrrolidine-3-carboxamide

To a solution of tert-butyl (3S)-3-(((2S)-1-(((63S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)(methyl)carbamoyl)pyrrolidine-1-carboxylate (0.283 g, 300.68 μmol) in DCM (4 mL) was added TFA (980.59 mg, 8.60 mmol) at 0° C. The mixture was stirred at room temperature for 30 min. The mixture was adjusted to pH 8 with the addition of sat. aq. NaHCO₃. The reaction mixture was partitioned between DCM (15 mL) and H₂O (10 mL). The organic phase was separated, washed with brine (2×5 mL), dried over Na₂SO₄, filtered and concentrated under reduced pressure to give the product (0.18 g, 71% yield) as a solid, which was used directly in the next step. LCMS (ESI) m/z [M+H] calcd for C₄₅H₆₁N₈O₆S: 841.4; found: 841.4.

### Step 3: Synthesis of (3S)-1-((2R,3R)-3-cyclopropyl-1-((R)-p-tolylsulfinyl)aziridine-2-carbonyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide

To a solution of (3S)—N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (0.13 g, 154.56 μmol) in MeCN (5 mL) was added (2R,3R)-3-cyclopropyl-1-((R)-p-tolylsulfinyl)aziridine-2-carboxylic acid (102.53 mg, 386.41 μmol, Li), HATU (146.93 mg, 386.41 μmol) and DIPEA (49.94 mg, 386.41 μmol) at 0° C. The mixture was stirred at room temperature for 30 min. The reaction mixture was partitioned between DCM (15 mL) and H₂O (10 mL). The organic phase was separated, washed with brine (2×5 mL), dried over Na₂SO₄, filtered and concentrated under reduced pressure to give a residue. The residue was purified by silica gel column chromatography (50→100% EtOAc/pet. ether) to give the product (0.12 g, 71% yield) as a solid.

### Step 4: Synthesis of (3S)-1-((2R,3R)-3-cyclopropylaziridine-2-carbonyl)-N-((2S)-1-(((63S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)-pyridazinacycloundecaphane-4-yl)amino)-3-methyl-1-oxobutan-2-yl)-N-methylpyrrolidine-3-carboxamide

To a solution of (3S)-1-((2R,3R)-3-cyclopropyl-1-((R)-p-tolylsulfinyl)aziridine-2-carbonyl)-N-((2S)-1-(((63S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide (0.08 g, 73.50 μmol) in THE (4 mL) was added MeMgBr (1 M, 294.01 μL). The mixture was stirred at −78° C. for 1 h. The reaction mixture was partitioned between DCM (15 mL) and H₂O (10 mL). The organic phase was separated, washed with brine (2×5 mL), dried over Na₂SO₄, filtered and concentrated under reduced pressure to give a residue. The residue was purified by reverse phase column chromatography (30→60% MeCN/H₂O 0.1% NH₄HCO₃) to give the product (0.016 g, 23% yield) as a solid. LCMS (ESI) m/z [M+H] calcd for C₅₁H₆₈N₉O₇S:950.50; found: 950.5.

### Synthesis of (3R)-1-((2R,3R)-3-cyclopropylaziridine-2-carbonyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide

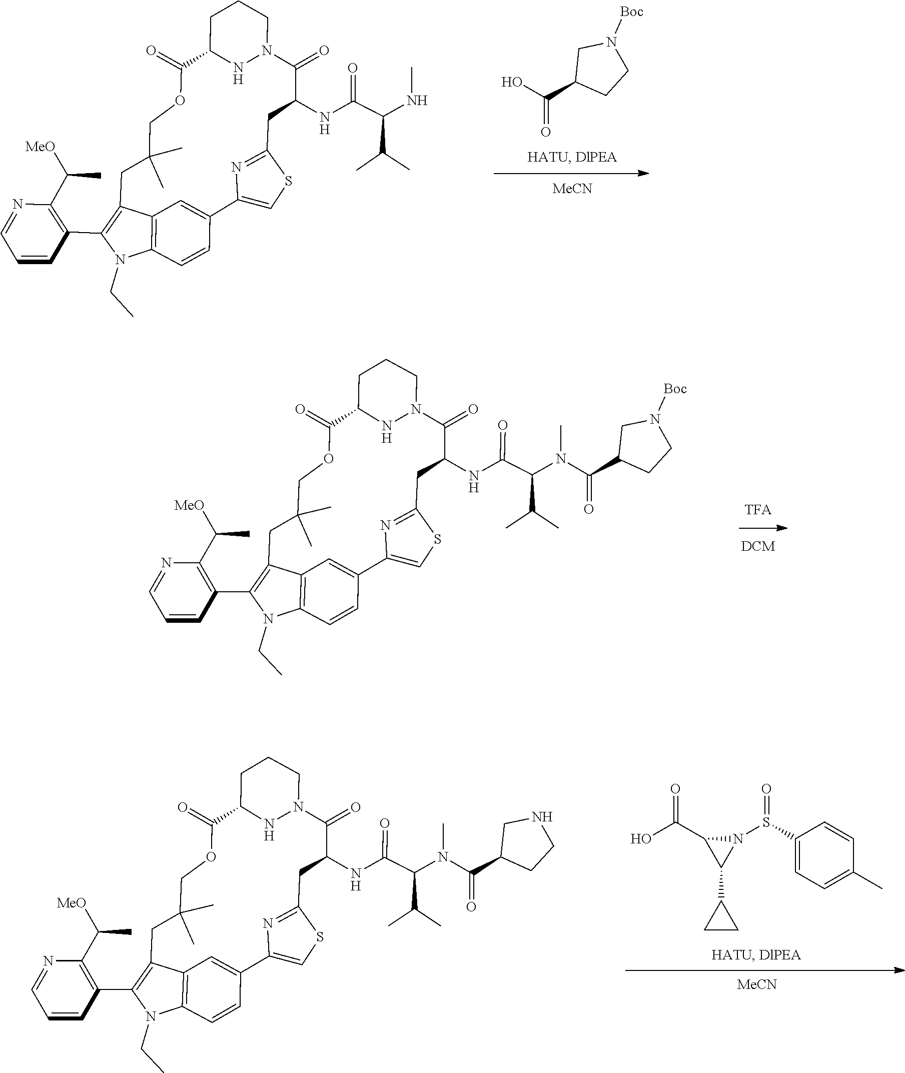

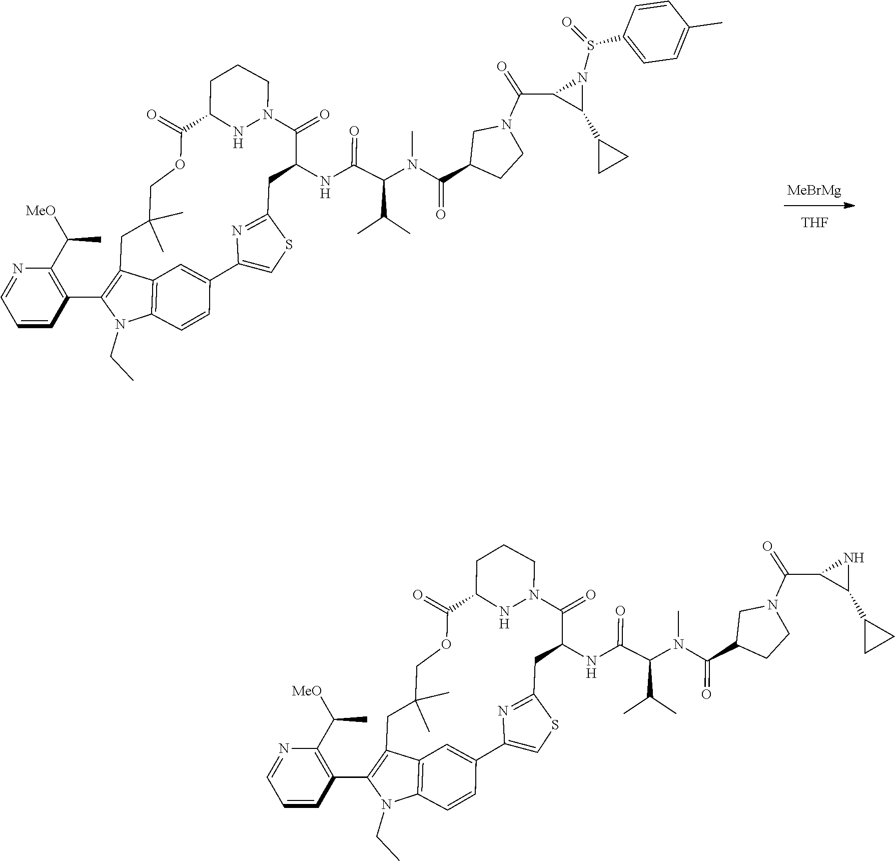

### Step 1: Synthesis of tert-butyl (3R)-3-(((2S)-1-(((63S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)(methyl)carbamoyl)pyrrolidine-1-carboxylate

To a solution of (2S)—N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-3-methyl-2-(methylamino)butanamide (200 mg, 268.83 μmol) in MeCN (3 mL) was added (3R)-1-tert-butoxycarbonylpyrrolidine-3-carboxylic acid (69.44 mg, 322.60 μmol), HATU (153.33 mg, 403.25 μmol) and DIPEA (104.23 mg, 806.50 μmol) at 0° C. and the mixture was stirred at room temperature for 30 min. The mixture was diluted with H₂O (5 mL) and extracted with DCM (3×5 mL). The combined organic layers were washed with brine (2×5 mL), dried over Na₂SO₄, filtered and the filtrate concentrated under reduced pressure to give a residue. The residue was purified by prep-TLC(10% MeOH/DCM) to give the product (180 mg, 71% yield) as a solid. LCMS (ESI) m/z [M+H] calcd for C₅₀H₆₉N₈O₈S: 941.5; found: 941.4.

### Step 2: Synthesis of (3R)—N-((2S)-1-(((63S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)-N-methylpyrrolidine-3-carboxamide

To a solution of tert-butyl (3R)-3-(((2S)-1-(((63S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)(methyl)carbamoyl)pyrrolidine-1-carboxylate (180 mg, 191.25 μmol) in DCM (2 mL) was added TFA (0.4 mL, 5.40 mmol) at 0° C. and the mixture was stirred at room temperature for 30 min. The mixture was adjusted to pH 8 with sat. aq. NaHCO₃. Then the mixture was diluted with H₂O (5 mL) and extracted with DCM (3×5 mL). The combined organic layers were washed with brine (2×5 mL), dried over Na₂SO₄, filtered and concentrated under reduced pressure to give the product (110 mg, 68% yield) as a solid. LCMS (ESI) m/z [M+H] calcd for C₄₅H₆₁N₈O₆S: 841.4; found: 841.5.

### Step 3: Synthesis of (3R)-1-((2R,3R)-3-cyclopropyl-1-((R)-p-tolylsulfinyl)aziridine-2-carbonyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-3-methyl-1-oxobutan-2-yl)-N-methylpyrrolidine-3-carboxamide

To a solution of (3R)—N-((2S)-1-(((63S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)amino)-3-methyl-1-oxobutan-2-yl)-N-methylpyrrolidine-3-carboxamide (110 mg, 130.79 μmol) in MeCN (2 mL) was added (2R,3R)-3-cyclopropyl-1-((R)-p-tolylsulfinyl)aziridine-2-carboxylic acid (89.02 mg, 326.96 μmol, Li), HATU (74.59 mg, 196.18 μmol) and DIPEA (50.71 mg, 392.36 μmol) at 0° C. and the mixture was stirred at room temperature for 30 min. The mixture was diluted with H₂O (5 mL) and extracted with DCM (3×5 mL). The combined organic layers were washed with brine (3×5 mL), dried over Na₂SO₄, filtered and concentrated under reduced pressure to give a residue. The residue was purified by prep-TLC (10% MeOH/DCM) to give the product (100 mg, 70% yield) as a solid. LCMS (ESI) m/z [M+H] calcd for C₅₈H₇₄N₉O₈S₂: 1088.5; found: 1088.6.

### Step 4: Synthesis of (3R)-1-((2R,3R)-3-cyclopropylaziridine-2-carbonyl)-N-((2S)-1-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)-pyridazinacycloundecaphane-4-yl)amino)-3-methyl-1-oxobutan-2-yl)-N-methylpyrrolidine-3-carboxamide

To a solution of (3R)-1-((2R,3R)-3-cyclopropyl-1-((R)-p-tolylsulfinyl)aziridine-2-carbonyl)-N-((2S)-1-(((63S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)-pyridazinacycloundecaphane-4-yl)amino)-3-methyl-1-oxobutan-2-yl)-N-methylpyrrolidine-3-carboxamide (140 mg, 128.63 μmol) in THE (1 mL) was added MeMgBr (1 M, 514.52 μL) at −78° C. and the mixture was stirred at room temperature for 30 min. The mixture was diluted with H₂O (2 mL) and extracted with DCM (3×2 mL). The combined organic layers were washed with brine (2×3 mL), dried over Na₂SO₄, filtered and the filtrate concentrated under reduced pressure to give a residue. The residue was purified by reverse phase chromatography (30→60% MeCN/H₂O 0.1% NH₄HCO₃) to give the product as a solid. LCMS (ESI) m/z [M+H] calcd for C₅₁H₆₈N₉O₇S: 950.50; found: 950.5.

### Synthesis of (2S,3R)—N-((1S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-1-((2R,3R)-3-cyclopropylaziridine-2-carbonyl)-N,2-dimethylpyrrolidine-3-carboxamide

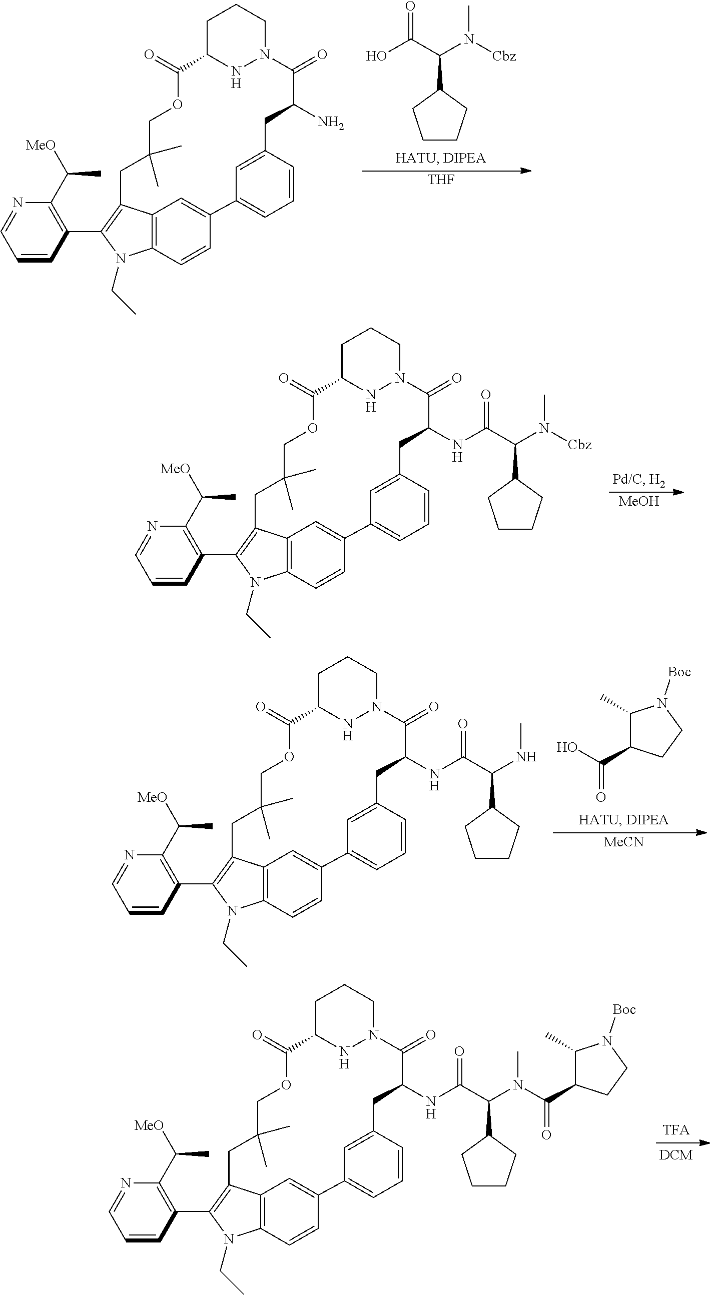

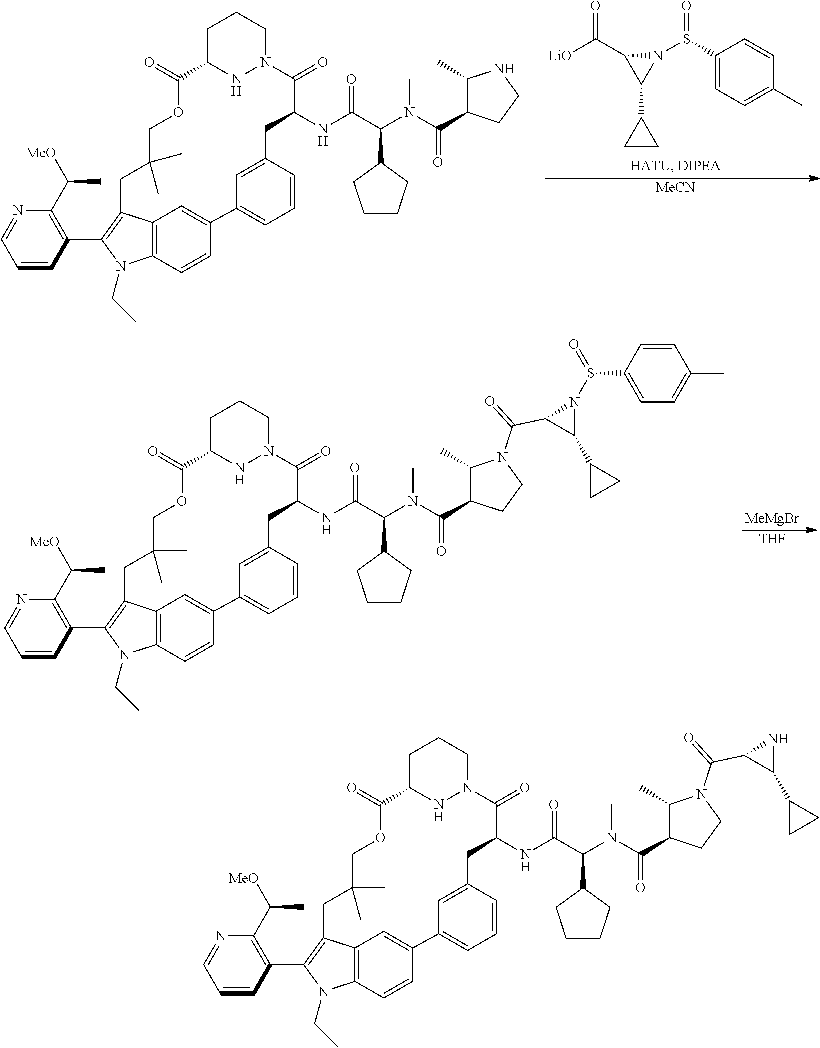

### Step 1: Synthesis of benzyl ((1S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)carbamate

To a solution of (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (2.56 g, 3.18 mmol) and (S)-2-(((benzyloxy)carbonyl)(methyl)amino)-2-cyclopentylacetic acid (1.11 g, 3.81 mmol) in THE (21.2 mL) at 0° C. was added DIPEA (2.76 mL, 15.9 mmol) and HATU (1.57 g, 4.13 mmol). After 2 h the resulting mixture was warmed to room temperature overnight and then diluted with EtOAc and H₂O. The aqueous layer was extracted with EtOAc and the combined organic layers washed with 0.2 N citric acid, H₂O, sat NaHCO₃, brine, dried with MgSO₄, filtered, and concentrated under reduced pressure. Purified by silica gel column chromatography (0→75% EtOAc/hexanes) afforded the desired product (2.22 g, 78% yield). LCMS (ESI) m/z [M+H] calcd for C₅₃H₆₄N₆O₇: 897.49; found: 897.9.

### Step 2: Synthesis of (2S)-2-cyclopentyl-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)- benzenacycloundecaphane-4-yl)-2-(methylamino)acetamide

To a solution of benzyl ((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)carbamate (1.77 g, 1.97 mmol) in MeOH (19.7 mL) was added 10 wt % Pd/C (180 mg). The resulting mixture was stirred for 4 h at room temperature under a hydrogen atmosphere, filtered through Celite, and the filter cake washed with MeOH. The filtrate was concentrated under reduced pressure to afford the desired product (1.42 g, crude), which was used without purification. LCMS (ESI) m/z [M+H] calcd for C₄₅H₅₈N₆O₅: 763.46; found: 763.7.

### Step 3: Synthesis of tert-butyl (2S,3R)-3-(((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)carbamoyl)-2-methylpyrrolidine-1-carboxylate

To a solution of (2S)-2-cyclopentyl-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-2-(methylamino)acetamide (475 mg, 0.623 mmol), (2S,3R)-1-(tert-butoxycarbonyl)-2-methylpyrrolidine-3-carboxylic acid (185 mg, 0.809 mmol), and DIPEA (540 mL, 3.11 mmol) in MeCN (3.1 mL) at 0° C. was added HATU (331 mg, 0.871 mmol). After 1 h the resulting mixture was warmed to room temperature and after an additional 2 h diluted with EtOAc and H₂O. The organic phase was washed with 0.2 N citric acid, H₂O, sat NaHCO₃, brine, dried with MgSO₄, filtered, and concentrated under reduced pressure. Purification by silica gel column chromatography (0→77% EtOAc/hexanes) afforded the desired product (478 mg, 79% yield). LCMS (ESI) m/z [M+H] calcd for C₅₆H₇₅N₇O₈: 974.58; found: 974.9.

### Step 4: Synthesis of (2S,3R)—N-((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-N,2-dimethylpyrrolidine-3-carboxamide

To a solution of tert-butyl (2S,3R)-3-(((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)carbamoyl)-2-methylpyrrolidine-1-carboxylate (478 mg, 0.491 mmol) in DCM (4.89 mL) at 0° C. was added TFA (1.87 mL, 24.5 mmol). After 1 h the reaction was quenched by the addition of sat. aq. NaHCO₃ and solid NaHCO₃ and then diluted with H₂O. The aqueous layer was extracted with DCM and the combined organic layers were washed with brine, dried with MgSO₄, filtered, and concentrated under reduced pressure affording the desired product (445 mg, crude), which was used without further purification.

LCMS (ESI) m/z [M+H] calcd for C₅₁H₆₇N₇O₆: 874.53; found: 874.9.

### Step 5: Synthesis of (2S,3R)—N-((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)-1-((2R,3R)-3-cyclopropyl-1-((R)-p-tolylsulfinyl)aziridine-2-carbonyl)-N,2-dimethylpyrrolidine-3-carboxamide

To a solution of (2S,3R)—N-((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)-N,2-dimethylpyrrolidine-3-carboxamide (440 mg, 0.503 mmol), lithium (2R,3R)-3-cyclopropyl-1-((R)-p-tolylsulfinyl)aziridine-2-carboxylate (177 mg, 0.654 mmol) and DIPEA (260 mL, 1.50 mmol) in MeCN (2.51 mL) at 0° C. was added HATU (286 mg, 0.754 mmol). Additional MeCN (2.0 mL) was added and the reaction was warmed to room temperature. After 1.5 h the reaction mixture was diluted with EtOAc and H₂O. The aqueous layer was extracted with EtOAc and the combined organic layers were washed with H₂O, sat NaHCO₃, and brine, dried with MgSO₄, filtered, and concentrated under reduced pressure. Purification by silica gel column chromatography (0→5% MeOH/DCM) afforded the desired product (425 mg, 75% yield). LCMS (ESI) m/z [M+H] calcd for C₆₄H₈₀N₈O₈S: 1121.59; found: 1121.9.

### Step 6: Synthesis (2S,3R)—N-((1S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-1-((2R,3R)-3-cyclopropylaziridine-2-carbonyl)-N,2-dimethylpyrrolidine-3-carboxamide

To a solution of (2S,3R)—N-((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)-1-((2R,3R)-3-cyclopropyl-1-((R)-p-tolylsulfinyl)aziridine-2-carbonyl)-N,2-dimethylpyrrolidine-3-carboxamide (425 mg, 0.379 mmol) in THE (7.55 mL) at −78° C. was added MeMgBr (667 mL, 2.27 mmol, 3.4 M in 2-MeTHF). After 30 min the reaction was quenched with the addition of sat. aq. NH₄Cl (7.0 mL), warmed to room temperature, and diluted with EtOAc and H₂O. The aqueous layer was extracted with EtOAc and the combined organic layers were washed with brine, dried (MgSO₄), filtered and concentrated under reduced pressure. Purification by reverse phase chromatography (10→99% MeCN/H₂O) afforded the desired product (181 mg, 49% yield). LCMS (ESI) m/z [M+H] calcd for C₅₇H₇₄N₈O₇: 983.58; found: 983.5.

### Synthesis of (3R,4S)—N-((1S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-1-((2R,3R)-3-cyclopropylaziridine-2-carbonyl)-N,4-dimethylpyrrolidine-3-carboxamide

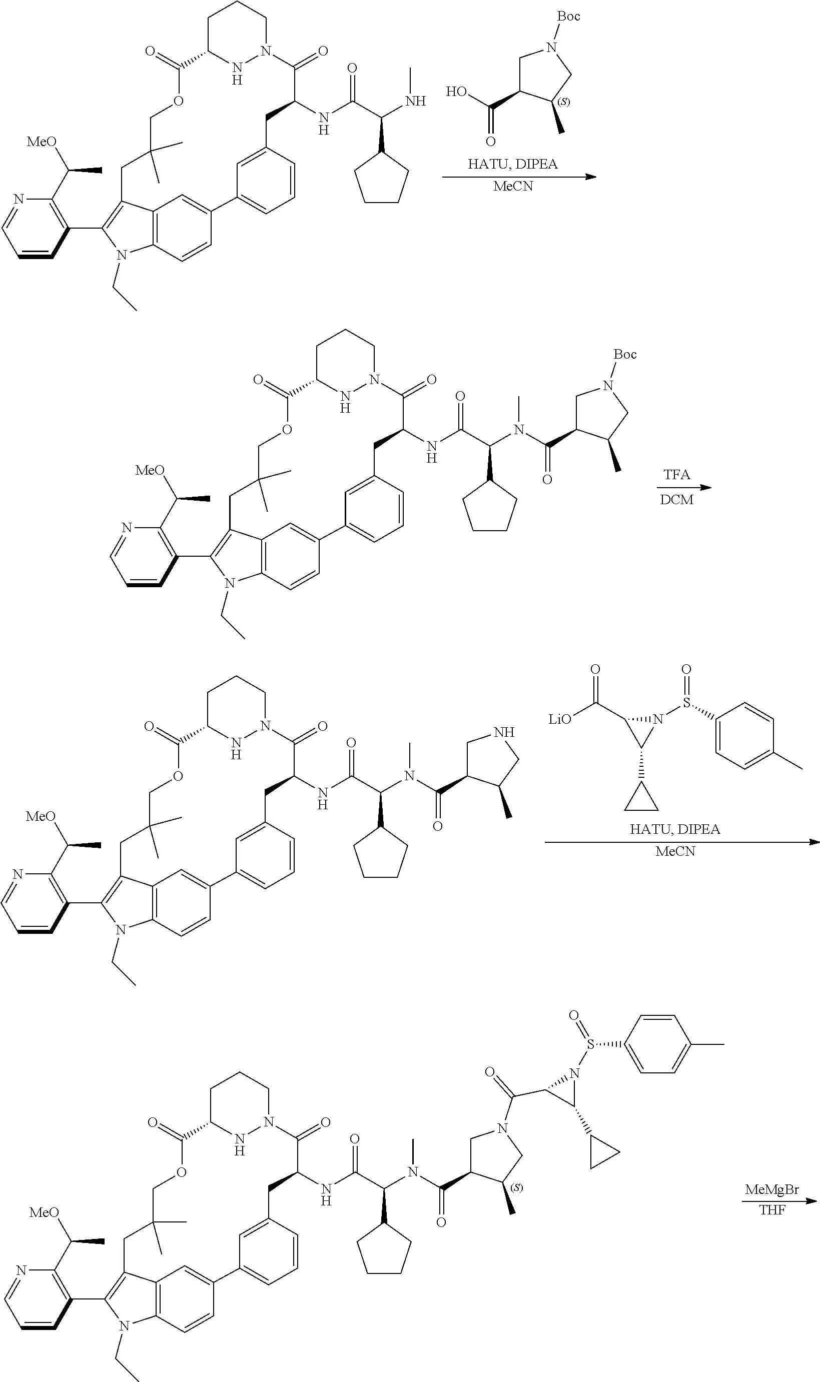

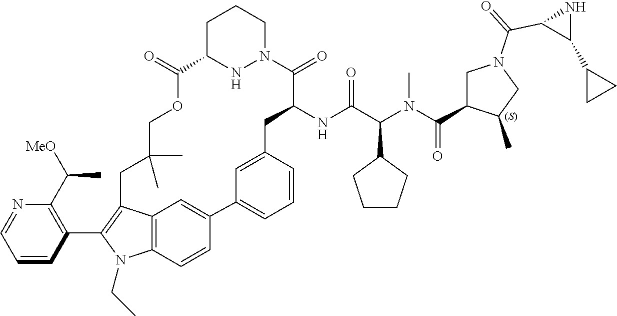

### Step 1: Synthesis of benzyl (3R,4S)-3-(((1S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)(methyl)carbamoyl)-4-methylpyrrolidine-1-carboxylate

To a solution of (2S)-2-cyclopentyl-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-2-(methylamino)acetamide (481 mg, 0.623 mmol), (3R,4S)-1-((benzyloxy)carbonyl)-4-methylpyrrolidine-3-carboxylic acid (215 mg, 0.817 mmol), and DIPEA (545 mL, 3.14 mmol) in MeCN (3.1 mL) at 0° C. was added HATU (334 mg, 0.880 mmol). After 1 h the resulting mixture was warmed to room temperature overnight and additional (3R,4S)-1-((benzyloxy)carbonyl)-4-methylpyrrolidine-3-carboxylic acid (49 mg, 0.186 mmol) and HATU (71 mg, 0.186 mmol) were added. After 4 h the reaction mixture was diluted with EtOAc and H₂O. The organic phase was washed with 0.2 N citric acid, H₂O, sat NaHCO₃, brine, dried with MgSO₄, filtered, and concentrated under reduced pressure. Purification by silica gel column chromatography (0→77% EtOAc/hexanes) afforded the desired product (379 mg, 60% yield). LCMS (ESI) m/z [M+H] calcd for C₅₉H₇₃N₇O₈: 1008.56; found: 1008.9.

### Step 2: Synthesis (3R,4S)—N-((1S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)-N,4-dimethylpyrrolidine-3-carboxamide

To a solution of benzyl (3R,4S)-3-(((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)carbamoyl)-4-methylpyrrolidine-1-carboxylate (366 mg, 0.366 mmol) in MeOH (18 mL) was added 10 wt % Pd/C (38 mg). The resulting mixture was stirred for 3 h at room temperature under a hydrogen atmosphere, filtered through Celite, and the filter cake washed with MeOH. The filtrate was concentrated under reduced pressure to afford the desired product (318 mg, crude), which was used without purification.

LCMS (ESI) m/z [M+H] calcd for C₅₁H₆₇N₇O₆: 874.53; found: 875.0.

### Step 3: Synthesis of (3R,4S)—N-((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-1-((2R,3R)-3-cyclopropyl-1-((R)-p-tolylsulfinyl)aziridine-2-carbonyl)-N,4-dimethylpyrrolidine-3-carboxamide

To a solution of (3R,4S)—N-((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)-N,4-dimethylpyrrolidine-3-carboxamide (318 mg, 0.364 mmol), lithium (2R,3R)-3-cyclopropyl-1-((R)-p-tolylsulfinyl)aziridine-2-carboxylate (128 mg, 0.472 mmol) and DIPEA (188 mL, 1.09 mmol) in MeCN (1.81 mL) at 0° C. was added HATU (207 mg, 0.545 mmol). Additional MeCN (2.0 mL) was added and the reaction was warmed to room temperature. After 16 h the reaction mixture was diluted with EtOAc and H₂O. The aqueous layer was extracted with EtOAc and the combined organic layers were washed with H₂O, sat NaHCO₃, and brine, dried with MgSO₄, filtered, and concentrated under reduced pressure. Purification by silica gel column chromatography (0→5% MeOH/DCM) afforded the desired product (203 mg, 50% yield). LCMS (ESI) m/z [M+H] calcd for C₆₄H₈₀N₈O₈S: 1121.59; found: 1121.6.

### Step 4: Synthesis of (3R,4S)—N-((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-1-((2R,3R)-3-cyclopropylaziridine-2-carbonyl)-N,4-dimethylpyrrolidine-3-carboxamide

To a solution of (3R,4S)—N-((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)-1-((2R,3R)-3-cyclopropyl-1-((R)-p-tolylsulfinyl)aziridine-2-carbonyl)-N,4-dimethylpyrrolidine-3-carboxamide (202 mg, 0.181 mmol) in THE (3.59 mL) at −78° C. was added MeMgBr (317 mL, 1.08 mmol, 3.4 M in 2-MeTHF). After 30 min the reaction was quenched with the addition of sat. aq. NH₄Cl (7.0 mL), warmed to room temperature, and diluted with EtOAc and H₂O. The aqueous layer was extracted with EtOAc and the combined organic layers were washed with brine, dried (MgSO₄), filtered and concentrated under reduced pressure. Purification by reverse phase chromatography (10→99% MeCN/H₂O) afforded the desired product (67 mg, 38% yield). LCMS (ESI) m/z [M+H] calcd for C₅₇H₇₄N₈O₇: 983.58; found: 983.9.

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

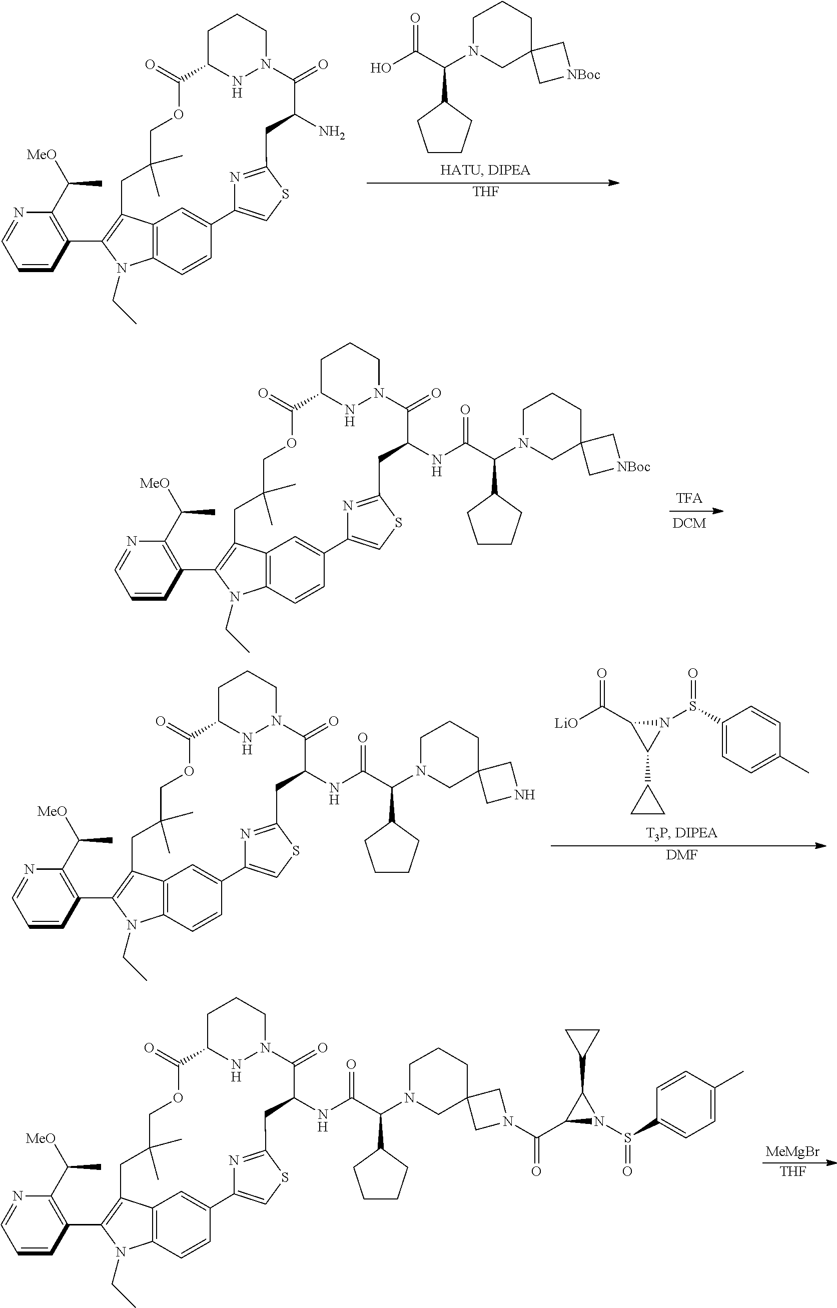

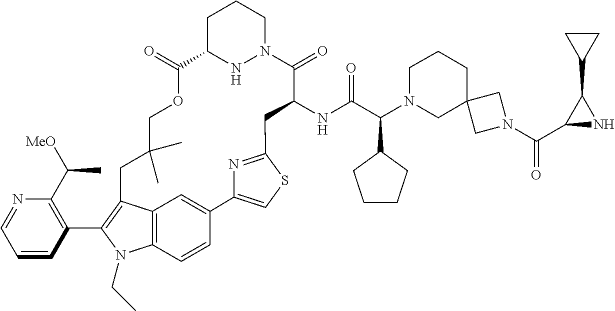

### Step 1: Synthesis of tert-butyl 6-((1S)-1-cyclopentyl-2-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,6-diazaspiro[3.5]nonane-2-carboxylate

To a solution of (S)-2-(2-(tert-butoxycarbonyl)-2,6-diazaspiro[3.5]nonan-6-yl)-2-cyclopentylacetic acid (363.20 mg, 1.03 mmol) in THE (5 mL) was added HATU (421.94 mg, 1.11 mmol) and DIPEA (307.32 mg, 2.38 mmol) at 0° C. followed by (6³S,4S,Z)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (500 mg, 792.65 μmol). The mixture was stirred at room temperature for 2 h. The reaction mixture was poured into cold sat. NH₄Cl (30 mL). The aqueous phase was extracted with EtOAc (3×20 mL). and the combined organic phases were washed with brine (20 mL), dried with anhydrous Na₂SO₄, filtered and concentrated under reduced pressure to give the product (1 g, crude) as solid, which was used directly in the next step. LCMS (ESI) m/z [M+H] calcd for C₅₃H₇₃N₈O₇S: 965.5; found: 965.6;

### Step 2: Synthesis of (2S)-2-cyclopentyl-N-((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)-2-(2,6-diazaspiro[3.5]nonan-6-yl)acetamide

To a solution of tert-butyl 6-((1 S)-1-cyclopentyl-2-(((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,6-diazaspiro[3.5]nonane-2-carboxylate (1 g, 808.08 μmol) in DCM (10 mL) was added TFA (1.84 g, 16.16 mmol) at 0° C., then the reaction was stirred at room temperature for 2 h. The reaction mixture was added dropwise to 100 mL sat. NaHCO₃ at 0° C. The mixture was extracted with DCM (3×30 mL). The combined organic phases were washed with brine (30 mL), dried with anhydrous Na₂SO₄, filtered and concentrated under reduced pressure to give (800 mg, 94% yield) as solid, which was used directly in the next step. LCMS (ESI) m/z [M+H] calcd for C₄₈H₆₅N₈₀₅S: 865.5; found: 865.5;

### Step 3: Synthesis of (2S)-2-cyclopentyl-2-(2-((2R,3R)-3-cyclopropyl-1-((R)-p-tolylsulfinyl)aziridine-2-carbonyl)-2,6-diazaspiro[3.5]nonan-6-yl)-N-((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)- pyridazinacycloundecaphane-4-yl)acetamide

To a solution of (2S)-2-cyclopentyl-N-((6³S,4S,2)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)-2-(2,6-diazaspiro[3.5]nonan-6-yl)acetamide (800 mg, 924.71 μmol) and (2R,3R)-3-cyclopropyl-1-((R)-p-tolylsulfinyl)aziridine-2-carboxylic acid (302.12 mg, 1.11 mmol, Li) in DMF (8 mL) was added DIPEA (836.57 mg, 6.47 mmol) and T3P (706.14 mg, 1.11 mmol). The reaction was stirred at room temperature for 1 h. The reaction was then poured into sat. NH₄Cl (100 mL). The aqueous phase was extracted with EtOAc (3×50 mL) and the combined organic phases were washed with brine (2×50 mL), dried with anhydrous Na₂SO₄, filtered and concentrated under reduced pressure to give crude product which was purified by silica gel column chromatography (30→100% EtOAc/pet. ether to 0→20% MeOH/EtOAc) to give the product (350 mg, 33.% yield) as a solid. LCMS (ESI) m/z [M+H] calcd for C₆₁H₇₈N₉O₇S₂: 1112.5; found: 1112.5

### Step 4: Synthesis of (2S)-2-cyclopentyl-2-(2-((2R,3R)-3-cyclopropylaziridine-2-carbonyl)-2,6-diazaspiro[3.5]nonan-6-yl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

To a solution of (2S)-2-cyclopentyl-2-(2-((2R,3R)-3-cyclopropyl-1-((R)-p-tolylsulfinyl)aziridine-2-carbonyl)-2,6-diazaspiro[3.5]nonan-6-yl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)acetamide (280 mg, 251.70 μmol) in THE (4.2 mL) was added MeMgBr (3 M, 671.19 μL) at −70° C. under N₂. The mixture was stirred at −70° C. for 1 h. The reaction mixture was quenched with sat. aq. NH₄Cl (30 mL) and then the aqueous phase was extracted with EtOAc (3×20 mL). The combined organic layers were washed with brine (10 mL), dried with anhydrous Na₂SO₄, filtered and concentrated under reduced pressure and purified by reverse phase chromatography (45→65% MeCN/H₂O 0.1% NH₄HCO₃) to give the product (69.17 mg, 28% yield) as solid. LCMS (ESI) m/z [M+H] calcd for C₅₄H₇₂N₉O₆S: 974.53; found: 974.5.

### Synthesis of (2S)-2-cyclopentyl-2-((R)-2-((2R,3R)-3-cyclopropylaziridine-2-carbonyl)-2,7-diazaspiro [4.5]decan-7-yl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

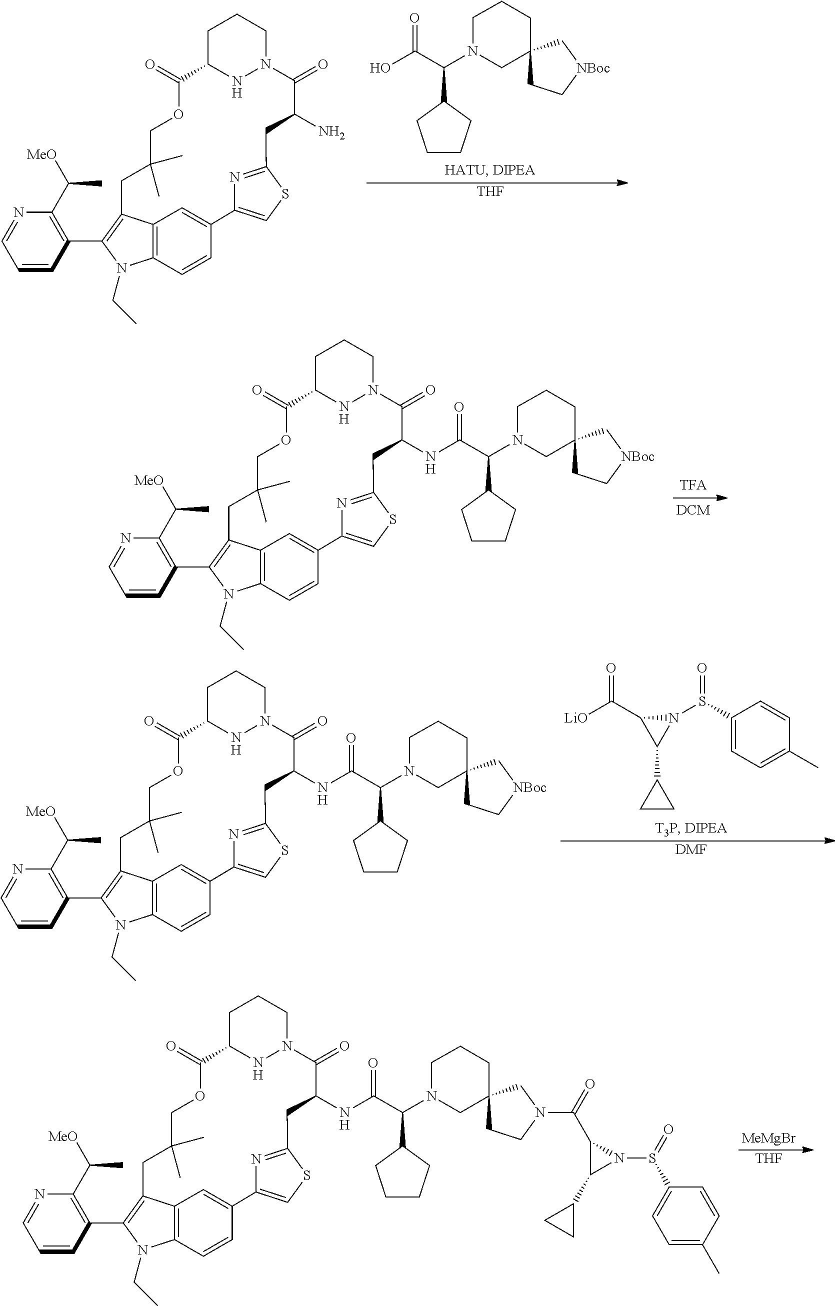

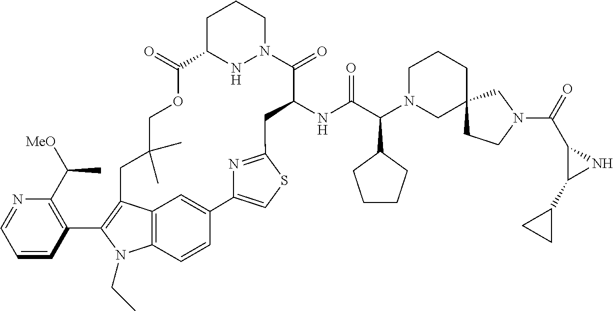

### Step 1: Synthesis of tert-butyl (5R)-7-((1S)-1-cyclopentyl-2-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.5]decane-2-carboxylate

To a solution of (S)-2-((R)-2-(tert-butoxycarbonyl)-2,7-diazaspiro[4.5]decan-7-yl)-2-cyclopentylacetic acid (270.16 mg, 737.16 μmol) in THE (3.1 mL) was added HATU (298.98 mg, 786.31 μmol) and DIPEA (254.06 mg, 1.97 mmol). The solution was stirred for 30 min and then (63S,4S,2)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (310 mg, 491.44 μmol) was added. Then mixture was stirred at room temperature for 1.5 h and then sat. NH₄Cl (10 mL) was added. The mixture was extracted with EtOAc (3×10 mL) and the combined organic layers were washed with brine (15 mL), dried over Na₂SO₄, filtered and concentrated under reduced pressure, then the crude product was purified by silica gel column chromatography (0→100% EtOAc/pet. ether) to give the product (420 mg, 87% yield) as solid. LCMS (ESI) m/z [M+H] calcd for C₅₄H₇₅N₈O₇S: 979.30; found: 979.5.

### Step 2: Synthesis of (2S)-2-cyclopentyl-N-((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)-2-((S)-2,7-diazaspiro[4.5]decan-7-yl) acetamide

To a solution of tert-butyl (5R)-7-((1 S)-1-cyclopentyl-2-(((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.5]decane-2-carboxylate (420 mg, 428.89 μmol) in DCM (4.2 mL) was added TFA (978.04 mg, 8.58 mmol) at 0° C., then the mixture was stirred at room temperature for 2 h. The reaction mixture was added dropwise to sat. NaHCO₃ (25 mL) at 0° C. Then the mixture was extracted with DCM (3×15 mL) and the combined organic phases were washed with brine (40 mL), dried with anhydrous Na₂SO₄, filtered and concentrated under reduced pressure to give the product (350 mg, crude) as a solid. LCMS (ESI) m/z [M+H] calcd for C₄₉H₆₇N₈₀₅S: 879.18; found: 879.5.

### Step 3: Synthesis of (2S)-2-cyclopentyl-2-((R)-2-((2R,3R)-3-cyclopropyl-1-((R)-p-tolylsulfinyl) aziridine-2-carbonyl)-2,7-diazaspiro[4.5]decan-7-yl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

To a solution of (2S)-2-cyclopentyl-N-((6³S,4S,2)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)-2-((S)-2,7-diazaspiro[4.5]decan-7-yl) acetamide (350 mg, 398.11 μmol) and (2R,3R)-3-cyclopropyl-1-[(R)-p-tolylsulfinyl] aziridine-2-carboxylic acid (216.78 mg, 796.21 μmol, Li) in DMF (3.5 mL) was added T3P (380.01 mg, 597.16 μmol) and DIPEA (411.61 mg, 3.18 mmol).

Then the mixture was stirred at room temperature for 1 h. H₂O (15 mL) was added and the mixture was extracted with EtOAc (3×15 mL). The combined organic layers were washed with aq. NH₄Cl (25 mL), dried over Na₂SO₄, filtered and concentrated under reduced pressure. The crude product was purified by prep-TLC (10% EtOAc/pet. ether) to give the product (435 mg, crude) as a solid. LCMS (ESI) m/z [M+H] calcd for C₆₂H₈₀N₉O₇S₂: 1126.49; found: 1126.6.

### Step 4: Synthesis of (2S)-2-cyclopentyl-2-((R)-2-((2R,3R)-3-cyclopropylaziridine-2-carbonyl)-2,7-diazaspiro[4.5]decan-7-yl)-N-((6³S,4S,Z)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

To a solution of (2S)-2-cyclopentyl-2-((R)-2-((2R,3R)-3-cyclopropyl-1-((R)-p-tolylsulfinyl) aziridine-2-carbonyl)-2,7-diazaspiro[4.5]decan-7-yl)-N-((6³S,4S,Z)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)- pyridazinacycloundecaphane-4-yl)acetamide (435 mg, 386.16 μmol) in THE (4.5 mL) was added MeMgBr (3 M, 1.03 mL) at −70° C. and the mixture was stirred at −70° C. for 2 h. Sat. NH₄Cl (15 mL) was added dropwise at −70° C. and then the mixture was warmed to room temperature. EtOAc (3×15 mL) was added and the organic layer was separated and concentrated under reduced pressure. The residue was purified by reverse phase chromatography (50→80% MeCN/H₂O 0.1% NH₄HCO₃) to give the product (107 mg, 28% yield) as a solid. LCMS (ESI) m/z [M+H] calcd for C₅₅H₇₄N₉O₆S: 988.55; found: 988.5.

### Synthesis of (2S)-2-((S)-7-((R)-aziridine-2-carbonyl)-2,7-diazaspiro[4.4]nonan-2-yl)-2-cyclopentyl-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

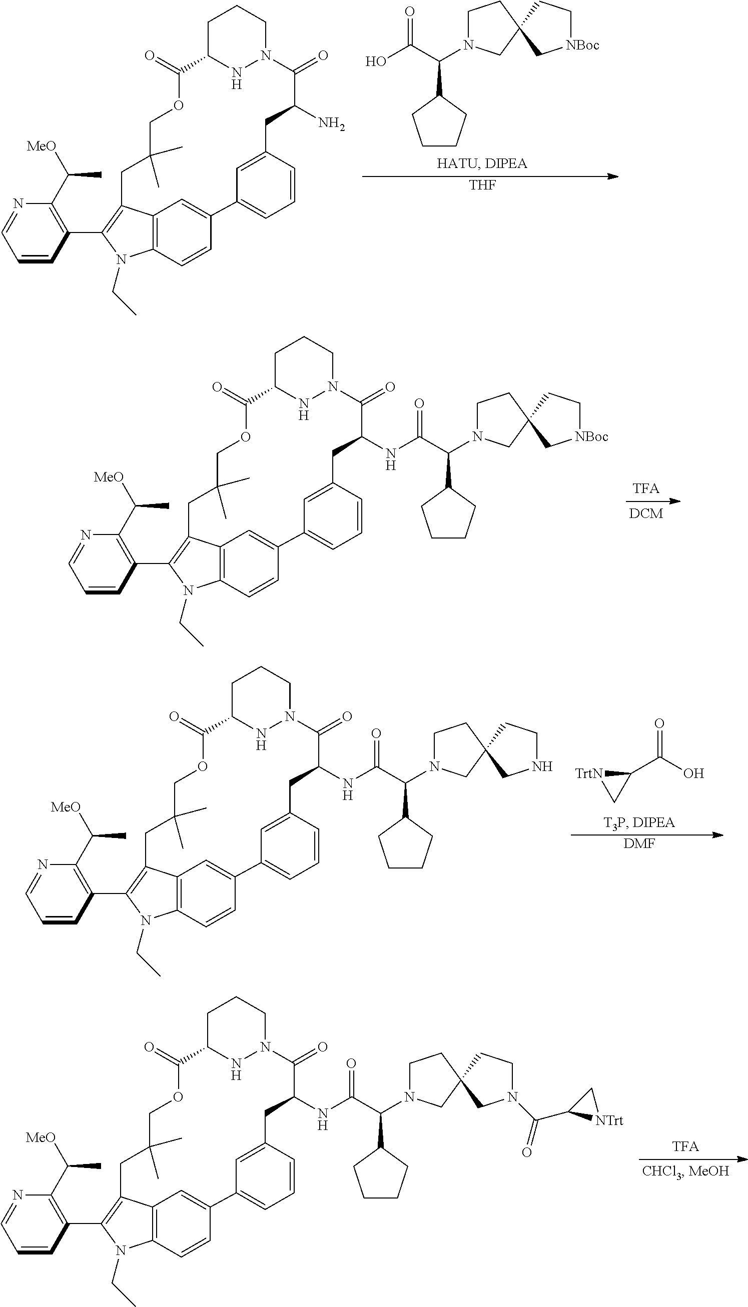

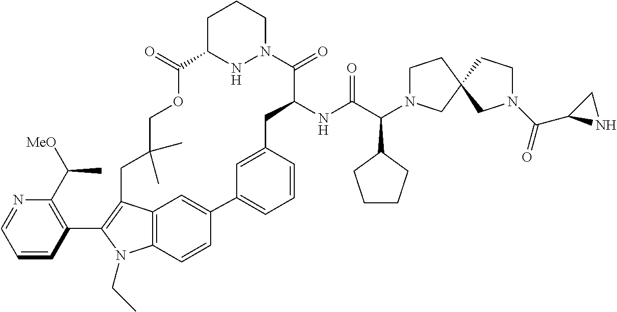

### Step 1: Synthesis of tert-butyl (5S)-7-((1S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)-2,7-diazaspiro[4.4]nonane-2-carboxylate

To a solution of (S)-2-((S)-7-(tert-butoxycarbonyl)-2,7-diazaspiro[4.4]nonan-2-yl)-2-cyclopentylacetic acid (609.69 mg, 1.73 mmol) and (6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (830 mg, 1.33 mmol) in THE (16 mL) was added HATU (708.30 mg, 1.86 mmol) and DIPEA (515.91 mg, 3.99 mmol) at 0° C., then the reaction was stirred at room temperature for 2.5 h. The residue was poured into cooled sat. NH₄Cl (50 mL), the aqueous phase was extracted with EtOAc (3×20 mL), and the combined organic phases were washed with brine (30 mL), dried with anhydrous Na₂SO₄, filtered and concentrated under reduced pressure. The crude product was purified by silica gel column chromatography (100% EtOAc/pet. ether) to give the product (1.2 g, 94% yield,) as a solid, which was used directly in the next step. LCMS (ESI) m/z [M+H] calcd for C₅₆H₇₆N₇O₇: 958.6; found: 958.7.

### Step 2: Synthesis of (2S)-2-cyclopentyl-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-2-((S)-2,7-diazaspiro[4.4]nonan-2-yl)acetamide

To a solution of tert-butyl (5S)-7-((1 S)-1-cyclopentyl-2-(((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (1.2 g, 1.25 mmol) in DCM (12 mL) was added TFA (2.86 g, 25.05 mmol). The mixture was stirred at 0° C. and was then warmed to room temperature and stirred for 2 h. The mixture was poured into cold sat. NaHCO₃ (40 mL) and the aqueous phase was extracted with DCM (3×20 mL). The combined organic phases were washed with brine (30 mL), dried with anhydrous Na₂SO₄, filtered and concentrated under reduced pressure the afford the product (850 mg, crude) as a solid, which was used directly in the next step. LCMS (ESI) m/z [M+H] calcd for C₅₁H₆₈N₇O₅: 858.5; found: 858.6.

### Step 3: Synthesis of (2S)-2-cyclopentyl-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-2-((S)-7-((R)-1-tritylaziridine-2-carbonyl)-2,7-diazaspiro[4.4]nonan-2-yl)acetamide

To a solution of (R)-1-tritylaziridine-2-carboxylic acid (438.69 mg, 932.27 μmol) and (2S)-2-cyclopentyl-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (400 mg, 466.14 μmol) in DMF (4 mL) was added DIPEA (421.71 mg, 3.26 mmol) and T3P (444.95 mg, 699.20 μmol) at 0° C., then the reaction was stirred at room temperature for 2 h. The residue was poured into cooled sat. NH₄Cl (40 mL), the aqueous phase was extracted with EtOAc (3×20 mL), the combined organic phases were washed with brine (30 mL), dried with anhydrous Na₂SO₄, filtered and concentrated under reduced pressure. The crude product was purified by silica gel column chromatography (30→100% EtOAc/pet. ether) to give the product (300 mg, 55% yield) as a solid. LCMS (ESI) m/z [M+H] calcd for C₇₃H₈₅N₈O₆: 1169.7; found: 1169.8.

### Step 4: Synthesis of (2S)-2-((S)-7-((R)-aziridine-2-carbonyl)-2,7-diazaspiro[4.4]nonan-2-yl)-2-cyclopentyl-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-cyclopentyl-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-2-((S)-7-((R)-1-tritylaziridine-2-carbonyl)-2,7-diazaspiro[4.4]nonan-2-yl)acetamide (300 mg, 256.52 μmol) in MeOH (2 mL) and CHCl₃ (1 mL) was added TFA (1.75 g, 15.39 mmol). The reaction mixture was stirred at 0° C. for 1 h and was the n added to sat. NaHCO₃ (30 mL). The aqueous phase was extracted with DCM (3×10 mL) and the combined organic phases were washed with brine (20 mL), dried with anhydrous Na₂SO₄, filtered and concentrated under reduced pressure. The crude product was purified by reverse phase chromatography (45→65% MeCN/H₂O 0.1% NH₄HCO₃) followed by a second purification by reverse phase chromatography (55→75% MeCN/H₂O 0.1% NH₄HCO₃) to give the product (55.34 mg, 23% yield) as a solid. LCMS (ESI) m/z [M+H] calcd for C₅₄H₇₁N₈O₆: 927.55; found: 927.6.

### Synthesis of (2S)-2-cyclopentyl-2-((S)-7-((2R,3R)-3-cyclopropyl-1-methylaziridine-2-carbonyl)-1-oxo-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

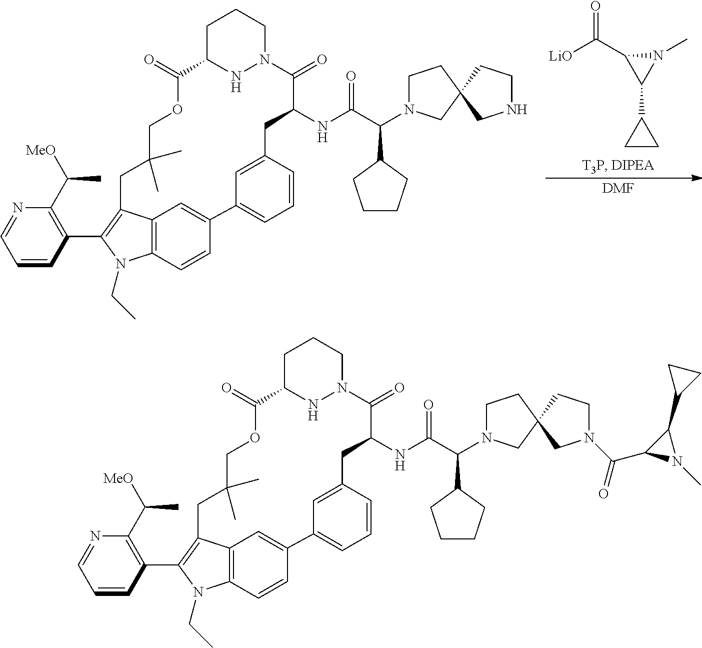

To a solution of (2S)-2-cyclopentyl-N-((6³S,4S)-1¹-ethyl-1²-(2-((S)-1-methoxyethyl)pyridine-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-2-((S)-1-oxo-2,7-diazaspiro[4.4]nonan-2-yl)acetamide (200 mg, 229.33 μmol) in DMF (2 mL) was added (2R,3R)-3-cyclopropyl-1-methylaziridine-2-carboxylic acid (40.76 mg, 275.20 μmol, Li), DIPEA (296.39 mg, 2.29 mmol) and T3P (189.72 mg, 298.13 μmol). The reaction mixture was stirred at room temperature for 1 h, was then poured into sat. NH₄Cl (10 mL), extracted with DCM (3×10 mL), and the combined organic phases were washed with brine (15 mL), dried with anhydrous Na₂SO₄, filtered and concentrated under reduced pressure. The residue was purified by reverse phase chromatography (45→65% MeCN/H₂O 0.1% NH₄HCO₃) to give the product (75.89 mg, 33% yield) as a solid. LCMS (ESI) m/z [M+H] calcd for C₅₈H₇₅N₈O₇: 995.58; found: 995.5.

### Synthesis of (2S)-2-cyclopentyl-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-7-((R)-oxirane-2-carbonyl)-2,7-diazaspiro[4.4]nonan-2-yl)acetamide

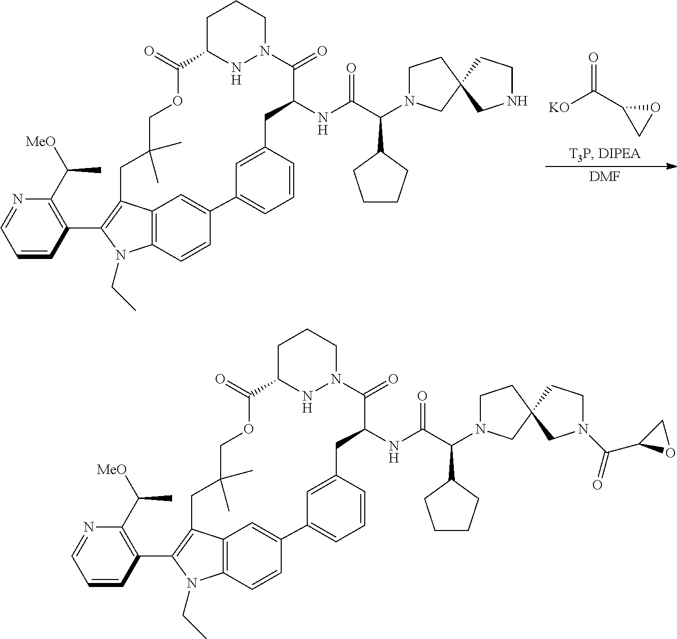

To a solution of (R)-oxirane-2-carboxylic acid (103.73 mg, 815.74 μmol, K) and (2S)-2-cyclopentyl-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (350 mg, 407.87 μmol) in DMF (3.5 mL) was added DIPEA (316.29 mg, 2.45 mmol) and T3P (311.46 mg, 489.44 μmol) at 0° C., then the reaction was stirred at room temperature for 2 h. The residue was poured into cold sat. NH₄Cl (30 mL), the aqueous phase was extracted with EtOAc (3×10 mL), and the combined organic phases were washed with brine (10 mL), dried with anhydrous Na₂SO₄, filtered and concentrated under reduced pressure. The crude product was purified by reverse phase chromatography (45→65% MeCN/H₂O 0.1% NH₄HCO₃) to give the product (68.44 mg, 17% yield) as a solid. LCMS (ESI) m/z [M+H] calcd for C₅₄H₇₀N₇O₇: 928.54; found: 928.5.

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

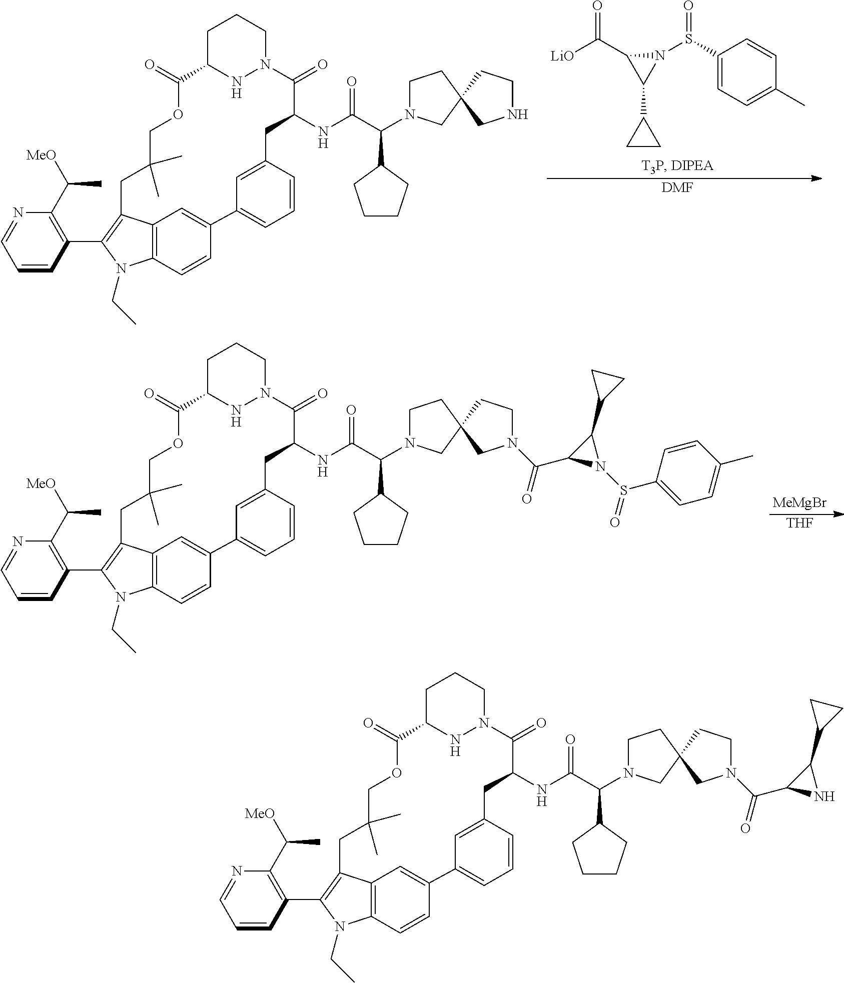

### Step 1: Synthesis of (2S)-2-cyclopentyl-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-cyclopentyl-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-2-((S)-2,7-diazaspiro[4.4]nonan-2-yl)acetamide (252.65 mg, 751.64 μmol, Li) in DMF (4.5 mL) was added DIPEA (323.81 mg, 2.51 mmol) and T3P (350.77 mg, 551.21 μmol) at 0° C., then the mixture was stirred at room temperature for 1 h. The reaction mixture was quenched with H₂O (10 mL) at 0° C. and then extracted with EtOAc (3×10 mL). The combined organic layers were washed with brine (3×10 mL), dried over Na₂SO₄, filtered and concentrated under reduced pressure. The residue was purified by silica gel column chromatography (0→100% EtOAc/pet. ether) to give the product (370 mg, 67% yield) as a solid. LCMS (ESI) m/z [M+H] calcd for C₆₄H₈₁N₈O₇S:1105.59, found: 1105.7.

### Step 2: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-cyclopentyl-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)acetamide (190 mg, 171.88 μmol) in THE (2 mL) was added MeMgBr (3 M, 458.34 μL) at −70° C., then the mixture was stirred at −70° C. for 1 h. Sat. NH₄Cl (10 mL) was added to the reaction mixture, dropwise, at −70° C. The cooling bath was removed, and the reaction mixture warmed to room temperature. The mixture was extracted with EtOAc (3×20 mL) and the combined organic layers were concentrated under reduced pressure. The residue was purified by reverse phase chromatography (45→75% MeCN/H₂O 0.1% NH₄HCO₃) to give the product (72.17 mg, 43% yield) as solid. LCMS (ESI) m/z [M+H] calcd for C₅₇H₇₅N₈O₆:967.58; found: 967.5.

### Synthesis of (2S)-2-((S)-7-((2R,3R)-3-cyclopropylaziridine-2-carbonyl)-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3) benzenacycloundecaphane-4-yl)-3-methylbutanamide

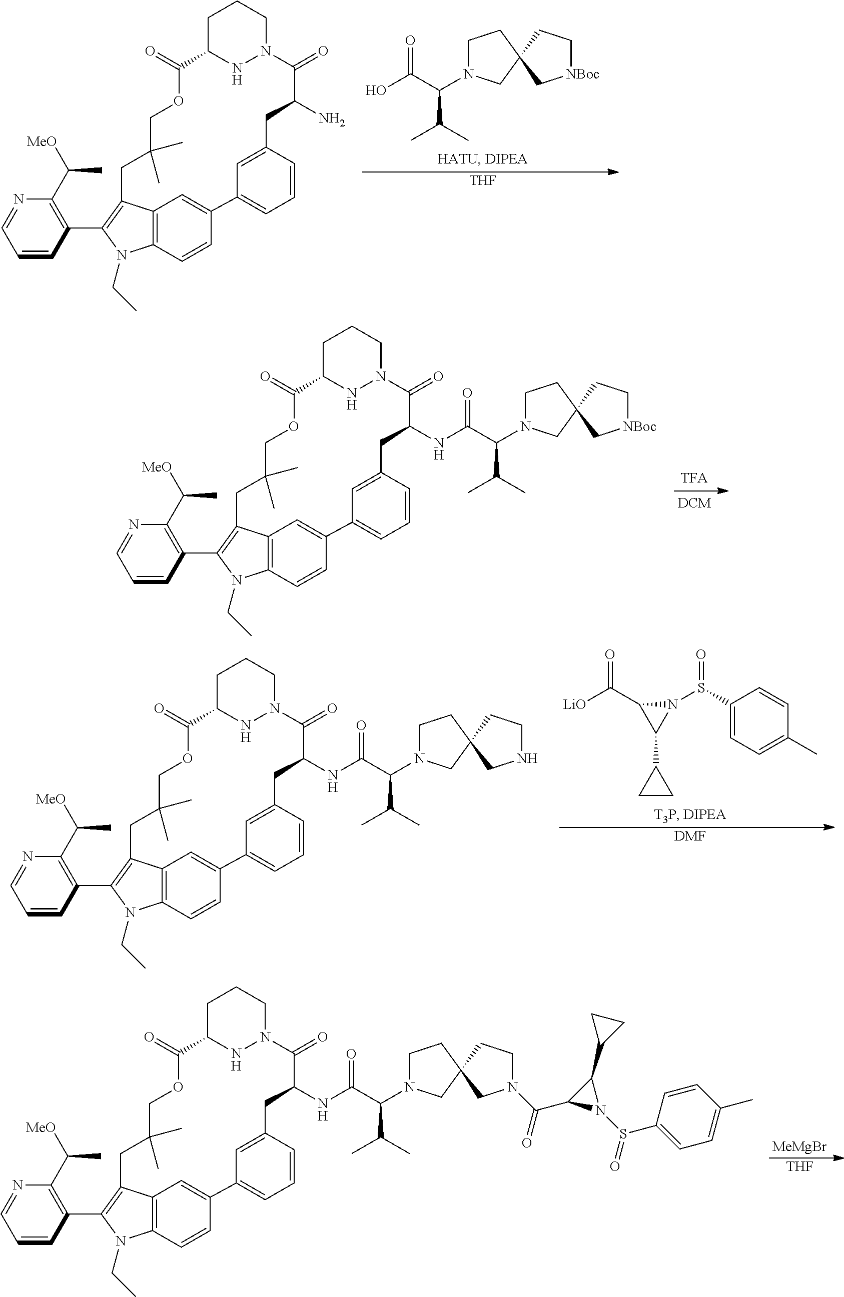

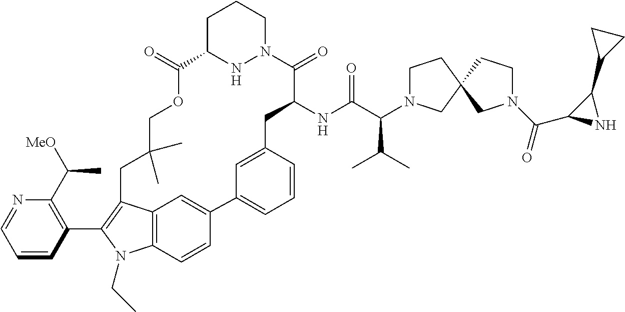

### Step 1: Synthesis of tert-butyl (5S)-7-((2S)-1-(((63S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-2,7-diazaspiro[4.4]nonane-2-carboxylate

To a solution of (S)-2-((S)-7-(tert-butoxycarbonyl)-2,7-diazaspiro[4.4]nonan-2-yl)-3-methylbutanoic acid (289.81 mg, 887.81 μmol) in THF (3 mL) was added DIPEA (176.52 mg, 1.37 mmol) and HATU (272.65 mg, 717.08 μmol), then the mixture was stirred for 20 min, followed by the addition of (63S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (426 mg, 682.93 μmol). The mixture was stirred at room temperature for 2 h, then sat. NH₄Cl (20 mL) was added. The solution was extracted with EtOAc (3×10 mL) and the combined organic layers were washed with brine (30 mL), dried over Na₂SO₄, filtered and concentrated under reduced pressure to give the product (636 mg, crude) as a solid, which was used directly in the next step. LCMS (ESI) m/z [M+H] calcd for C₅₄H₇₄N₇O₇:932.56; found: 932.6.

### Step 2: Synthesis of (2S)—N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-((S)-2,7-diazaspiro[4.4]nonan-2-yl)butanamide

To a solution of tert-butyl (5S)-7-((2S)-1-(((63S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-3-methyl-1-oxobutan-2-yl)-2,7-diazaspiro[4.4]nonane-2-carboxylate (857 mg, 919.33 μmol) in DCM (9.3 mL) was added TFA (2.10 mg, 18.39 mmol) at 0° C. Then the mixture was stirred at room temperature for 3.5 h. The reaction mixture was added dropwise to sat. NaHCO₃ (30 mL) at 0° C. The mixture was extracted with DCM (3×10 mL) and the combined organic phases were washed with brine (50 mL), dried with anhydrous Na₂SO₄, filtered and concentrated under reduced pressure to give the product (764 mg, crude) as a solid. LCMS (ESI) m/z [M+H] calcd for C₄₉H₆₆N₇O₅: 832.50; found: 832.5.

### Step 3: Synthesis of (2S)-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methylbutanamide

To a solution of (2R,3R)-3-cyclopropyl-1-((R)-p-tolylsulfinyl)aziridine-2-carboxylate (89.98 mg, 331.70 μmol Li) and (2S)—N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-((S)-2,7-diazaspiro[4.4]nonan-2-yl)butanamide (184 mg, 221.13 μmol) in DMF (1.9 mL) was added DIPEA (114.32 mg, 884.53 μmol) and T3P (154.79 mg, 243.25 μmol). Then the mixture was stirred at room temperature for 1 h. Sat. NH₄Cl (10 mL) was added and the mixture was extracted with EtOAc (3×10 mL). The combined organic layers were washed with brine (20 mL), dried over anhydrous Na₂SO₄, filtered and concentrated under reduced pressure to give the crude product. The crude product was purified by silica gel column chromatography (0→100% MeOH/EtOAc) to give the product (170 mg, 71% yield) as a solid. LCMS (ESI) m/z [M/2+H] calcd for C₆₂H₇₈N₈O₇S/2+H: 540.78; found: 540.7.

### Step 4: Synthesis of (2S)-2-((S)-7-((2R,3R)-3-cyclopropylaziridine-2-carbonyl)-2,7-diazaspiro [4.4]nonan-2-yl)-N-((6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a solution of (2S)-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-1¹-ethyl-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (255 mg, 236.24 μmol) in THE (2.6 mL) was added MeMgBr (3 M, 628.98 μL) at −70° C. Then the mixture was stirred at −70° C. for 1 h. Sat. NH₄Cl (20 mL) was added dropwise at −70° C. The cooling bath was removed, and the reaction mixture was warmed to room temperature. EtOAc (3×10 mL) was added and the organic layer was separated and concentrated under reduced pressure. The residue was purified by reverse phase chromatography (35→65% MeCN/H₂O 0.1% NH₄HCO₃) to give the product (71.7 mg, 32% yield) as a solid. LCMS (ESI) m/z [M+H] calcd for C₅₅H₇₃N₈O₆: 941.57; found: 941.6.

### Synthesis of (2S)-2-cyclopentyl-N-((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina- 2(5,1)-pyridinacycloundecaphane-4-yl)-2-((S)-7-((R)-oxirane-2-carbonyl)-2,7-diazaspiro[4.4]nonan-2-yl)acetamide

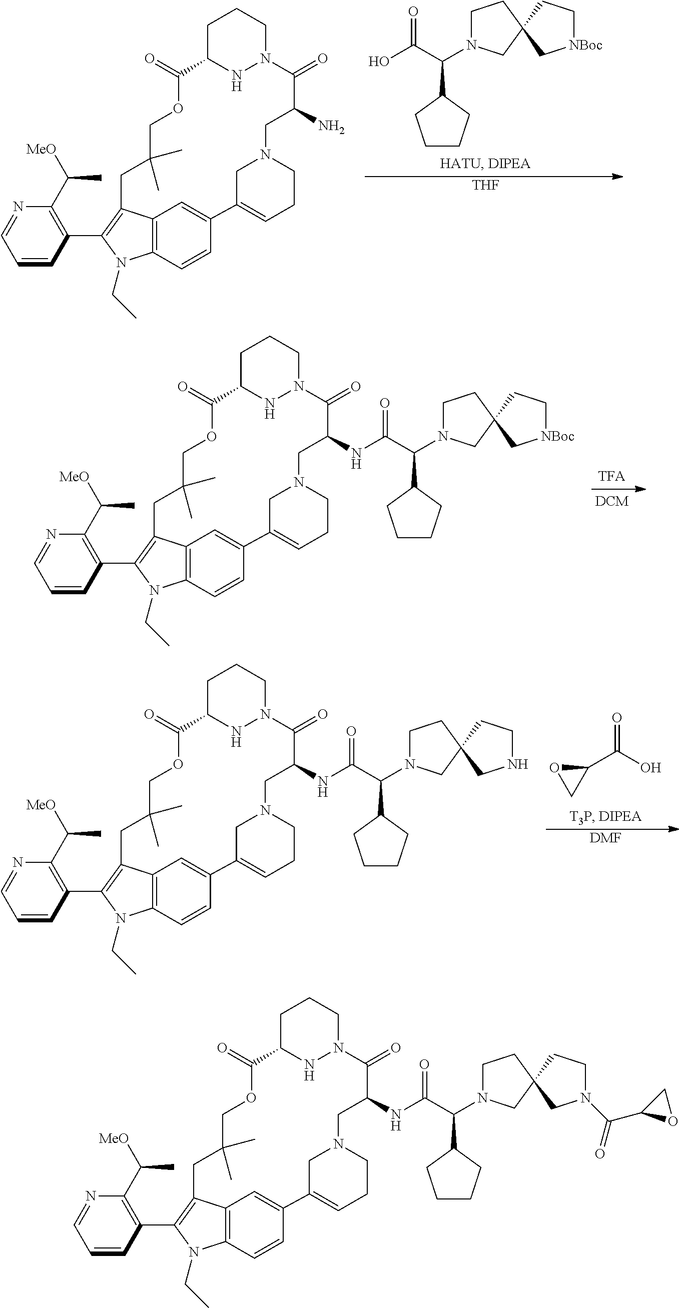

### Step 1: Synthesis of tert-butyl (5S)-7-((1 S)-1-cyclopentyl-2-(((6³S,4S)-1¹-ethyl-1²-(2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1′H-8-oxa-1(5,3)-indola-6(1,3)-pyridazina-2(5,1)-pyridinacycloundecaphane-4-yl)amino)-2-oxoethyl)-2,7-diazaspiro[4.4]nonane-2-carboxylate

To a solution of (S)-2-((S)-7-(tert-butoxycarbonyl)-2,7-diazaspiro[4.4]nonan-2-yl)-2-cyclopentylacetic acid (215.81 mg, 612.28 μmol) in MeCN (3.5 mL) at 0° C. was added HATU (243.39 mg, 640.11 μmol) and DIPEA (179.84 mg, 1.39 mmol). The mixture was stirred for 30 min, followed by the addition of (6³S,4S)-4-amino-1¹-ethyl-1²-(2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-5,7-dione (350 mg, 556.61 μmol). The mixture was stirred at room temperature for 1 h. The reaction mixture was quenched by cold sat. NH₄Cl (50 mL), extracted with EtOAc (3×20 mL), and the combined organic layers were washed with brine (30 mL), dried over anhydrous Na₂SO₄, filtered and concentrated under reduced pressure to give the product (680 mg, crude) as a solid. LCMS (ESI) m/z [M+H] calcd for C₅₅H₇₉N₈O₇: 963.6; found: 963.7.

### Step 2: Synthesis of (2S)-2-cyclopentyl-N-((6³S,4S)-1¹-ethyl-1²-(2-((S)-1-methoxyethyl) pyridine-3-yl)-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)-2-((S)-2,7-diazaspiro[4.4]nonan-2-yl)acetamide

To a solution of tert-butyl (5S)-7-((1S)-1-cyclopentyl-2-(((6³S,4S)-1¹-ethyl-1²-(2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1′H-8-oxa-1(5,3)-indola-6(1,3)-pyridazina-2(5,1)-pyridinacycloundecaphane-4-yl)amino)-2-oxoethyl)-2,7-diazaspiro[4.4]nonane-2-carboxylate (680 mg, 705.94 μmol) in DCM (7 mL) was added TFA (2.41 g, 21.18 mmol) at 0° C. The mixture was stirred at room temperature for 1 h. The reaction mixture was poured into sat. NaHCO₃ (10 mL), extracted with DCM (3×3 mL) and the combined organic layers were washed with brine (10 mL), dried over anhydrous Na₂SO₄, filtered and concentrated under reduced pressure to give the product (510 mg, crude) as a solid, which was used directly in the next step. LCMS (ESI) m/z [M+H] calcd for C₅₀H₇₁N₈O₅: 863.5; found: 863.6.

### Step 3: Synthesis of (2S)-2-cyclopentyl-N-((6³S,4S)-1¹-ethyl-1²-(2-((S)-1-methoxyethyl) pyridine-3-yl)-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)-2-((S)-7-((R)-oxirane-2-carbonyl)-2,7-diazaspiro[4.4]nonan-2-yl)acetamide

To a solution of (R)-oxirane-2-carboxylic acid (147.32 mg, 1.16 mmol K) and (2S)-2-cyclopentyl-N-((6³S,4S)-1¹-ethyl-1²-(2-((S)-1-methoxyethyl) pyridine-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)-2-((S)-2,7-diazaspiro[4.4]nonan-2-yl)acetamide (500 mg, 579.28 μmol) in DMF (5 mL) was added DIPEA (748.66 mg, 5.79 mmol) and T3P (737.27 mg, 1.16 mmol). The mixture was stirred at room temperature for 1 h. The reaction mixture was quenched with H₂O (50 mL), extracted with EtOAc (3×15 mL) and the combined organic layers were washed with brine (30 mL), dried over anhydrous Na₂SO₄, filtered and concentrated under reduced pressure. The residue was purified by reverse phase chromatography (45→75% MeCN/H₂O 0.1% NH₄HCO₃) to give the product (184.4 mg, 33% yield) as a solid. LCMS (ESI) m/z [M+H] calcd for C₅₃H₇₃N₈O₇: 933.56; found: 933.6

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)acetamide

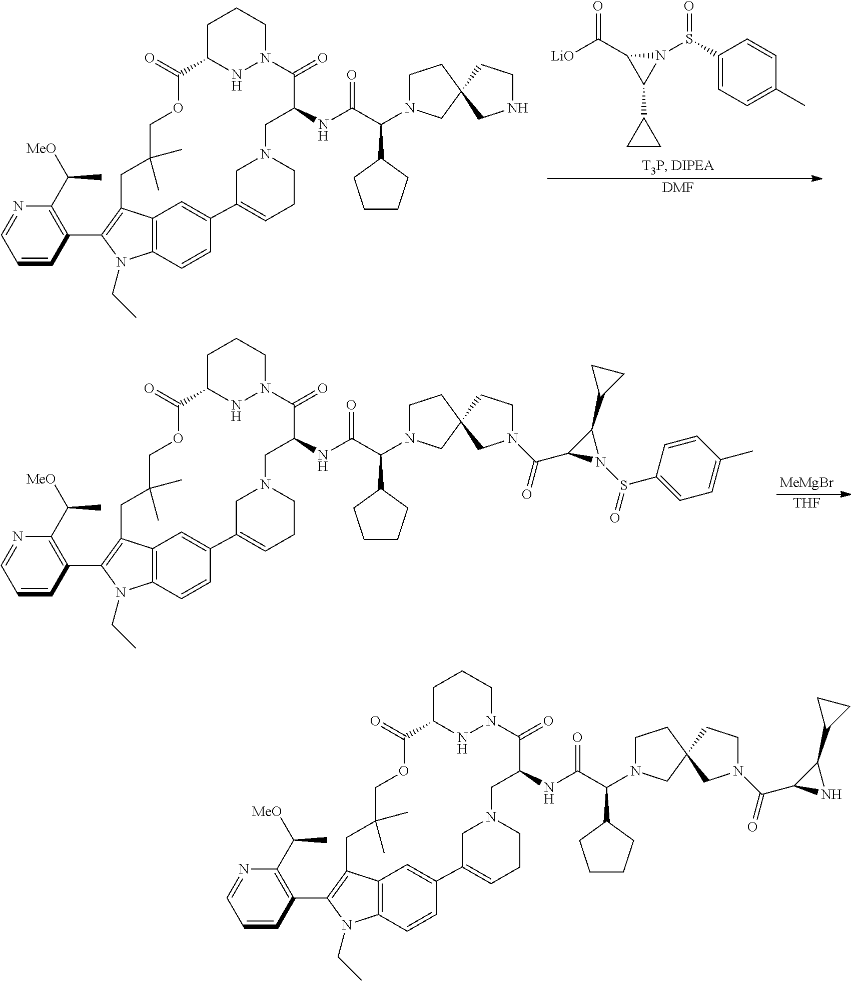

### Step 1: Synthesis of (2S)-2-cyclopentyl-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1′H-8-oxa-1(5,3)-indola-6(1,3)-pyridazina-2(5,1)-pyridinacycloundecaphane-4-yl)acetamide

To a solution of (2S)-2-cyclopentyl-N-((6³S,4S)-1¹-ethyl-1²-(2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina- 2(5,1)-pyridinacycloundecaphane-4-yl)-2-((S)-2,7-diazaspiro[4.4]nonan-2-yl)acetamide (400 mg, 463.42 μmol) and (2R,3R)-3-cyclopropyl-1-((R)-p-tolylsulfinyl) aziridine-2-carboxylic acid (252.35 mg, 926.85 μmol, Li) in DMF (4 mL) was added DIPEA (299.47 mg, 2.32 mmol) and T3P (294.91 mg, 463.42 μmol) at 0° C. The mixture was stirred at room temperature for 1 h. The reaction mixture was added to cold sat. NH₄Cl (20 mL). The aqueous phase was extracted with EtOAc (3×20 mL) and the combined organic layers were washed with brine (50 mL), dried with anhydrous Na₂SO₄, filtered and concentrated under reduced pressure. The crude product was purified by silica gel column chromatography (0→10% MeOH/EtOAc) to give the product (180 mg, 34% yield) as a solid. LCMS (ESI) m/z [M+H] calcd for C₆₃H₈₄N₉O₇S: 1110.61; found: 1110.8.

### Step 2: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)acetamide

To a solution of (2S)-2-cyclopentyl-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina- 2(5,1)-pyridinacycloundecaphane-4-yl)acetamide (200 mg, 180.11 μmol) in THE (2 mL) was added MeMgBr (3 M, 480.29 μL). The reaction mixture was stirred at −78° C. for 1 h. The reaction mixture was added to cold sat. NH₄Cl (20 mL). The aqueous phase was extracted with EtOAc (3×20 mL), and the combined organic layers were washed with brine (50 mL), dried with anhydrous Na₂SO₄, filtered and concentrated under reduced pressure. The product was purified by reverse phase chromatography (45→75% MeCN/H₂O 0.1% NH₄HCO₃) to give the product (66.36 mg, 38% yield) as a solid. LCMS: (ESI) m/z [M+H] calcd for C₅₆H₇₈N₉O₆:972.61; found: 972.5.

### Synthesis of (2S)-2-((S)-7-((2R,3R)-3-cyclopropylaziridine-2-carbonyl)-2,7-diazaspiro[4.4]nonan-2-yl)-N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

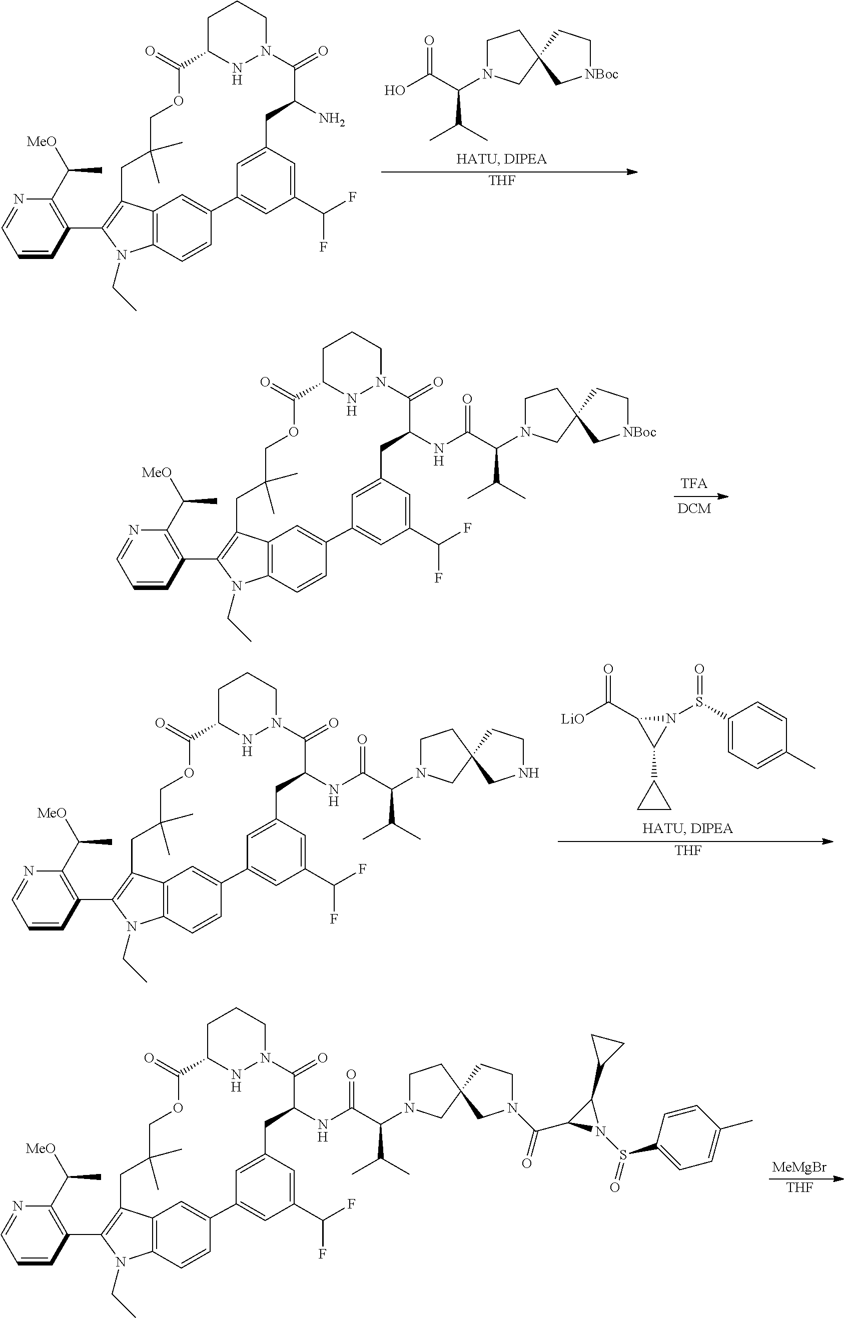

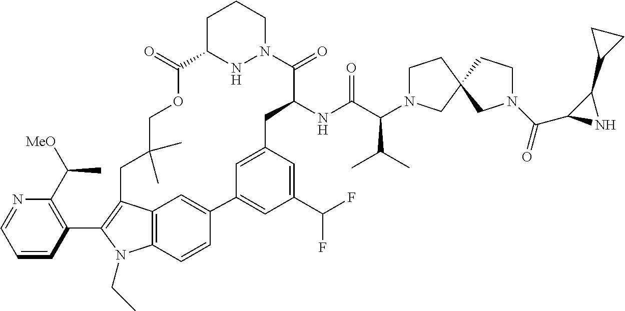

### Step 1: Synthesis of tert-butyl (5S)-7-((2S)-1-(((6³S,4S)-25-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-2,7-diazaspiro[4.4]nonane-2-carboxylate

To a solution of (S)-2-((S)-7-(tert-butoxycarbonyl)-2,7-diazaspiro[4.4]nonan-2-yl)-3-methylbutanoic acid (290.68 mg, 890.49 μmol) in THE (5 mL) was added HATU (338.59 mg, 890.49 μmol) and DIPEA (230.17 mg, 1.78 mmol) at 0° C. After stirring for 5 min, (6³S,4S)-4-amino-2⁵-(difluoromethyl)-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (0.4 g, 593.66 μmol) was added at 0° C. The reaction mixture was stirred for 30 min at room temperature. The resulting mixture was diluted with EtOAc (30 mL) and quenched with H₂O (20 mL). Then the aqueous layer was extracted with EtOAc 120 mL (4×30 mL). The organic layers were combined, washed with brine (10 mL), and dried over Na₂SO₄. The solvent was concentrated under reduced pressure to give the product (0.5 g, crude) as an oil, which was used directly in the next step. LCMS (ESI) m/z [M+H] calcd for C₅₅H₇₄F₂N₇O₇: 982.5; found: 982.5.

### Step 2: Synthesis of (2S)—N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)-3-methyl-2-((S)-2,7-diazaspiro[4.4]nonan-2-yl)butanamide

To a solution of tert-butyl (5R)-7-((2S)-1-(((6³S,4S)-25-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-2,7-diazaspiro[4.4]nonane-2-carboxylate (0.5 g, 509.06 μmol) in DCM (10 mL) was added TFA (3.85 g, 33.77 mmol) at room temperature. The resulting mixture was stirred at room temperature for 2 h. The solvent was removed under reduced pressure and the residue was quenched with H₂O (15 mL). The aqueous layer was extracted with MTBE (2×15 mL). The aqueous layer was then treated with sat. aq. NaHCO₃ to pH 7˜8, then extracted with EtOAc (5×20 mL). The organic layers were combined, washed with brine (10 mL), and dried over Na₂SO₄. The solvent was removed under reduced pressure to give the product (0.45 g, crude) as a solid, which was used directly in the next step. LCMS (ESI) m/z [M+H] calcd for C₅₀H₆₆F₂N₇O₅:882.50; found: 882.4.

### Step 3: Synthesis of (2S)-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)-3-methylbutanamide

To a solution of (2R,3R)-3-cyclopropyl-1-((R)-p-tolylsulfinyl)aziridine-2-carboxylic acid (270.71 mg, 1.02 mmol) in THE (3 mL) was added DIPEA (197.80 mg, 1.53 mmol) and HATU (290.96 mg, 765.23 μmol) at 0° C. After stirring for 5 min, (2S)—N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)-3-methyl-2-((S)-2,7-diazaspiro[4.4]nonan-2-yl)butanamide (0.45 g, 510.15 μmol) was added at 0° C. The reaction mixture was stirred for 30 min at room temperature. The solvent was then removed under reduced pressure and the resulting mixture was diluted with EtOAc (15 mL) and washed with H₂O (15 mL). Then the aqueous layer was extracted with EtOAc (4×15 mL). The organic layers were combined, washed with brine (10 mL), and dried over Na₂SO₄. The solvent was removed under reduced pressure. The crude product was purified by silica gel column chromatography (0→10% MeOH/DCM) to give the product (0.4 g, 69% yield) as an oil. LCMS (ESI) m/z [M/2+H] calcd for C₆₃H₇₉F₂N₈O₇S/2+1:565.3; found: 565.7.

### Step 4: Synthesis of (2S)-2-((S)-7-((2R,3R)-3-cyclopropylaziridine-2-carbonyl)-2,7-diazaspiro[4.4]nonan-2-yl)-N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methylbutanamide

To a solution of (2S)-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (0.4 g, 354.17 μmol) in THE (5 mL) at −78° C. was added MeMgBr (3 M, 472.23 μL). The reaction mixture was stirred for 30 min at −78° C. H₂O (10 mL) was then added to the reaction at 0° C. The aqueous layer was extracted with EtOAc (4×15 mL) and the organic layers were combined, washed with brine (10 mL), and dried over Na₂SO₄, filtered and concentrated under reduced pressure. The crude product was purified by reverse phase chromatography (40→70% MeCN/H₂O 0.1% NH₄HCO₃) to afford the product (0.094 g, 27% yield) as solid. LCMS (ESI) m/z [M+H] calcd for C₅₆H₇₃F₂N₈O₆:991.56; found: 992.2.

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

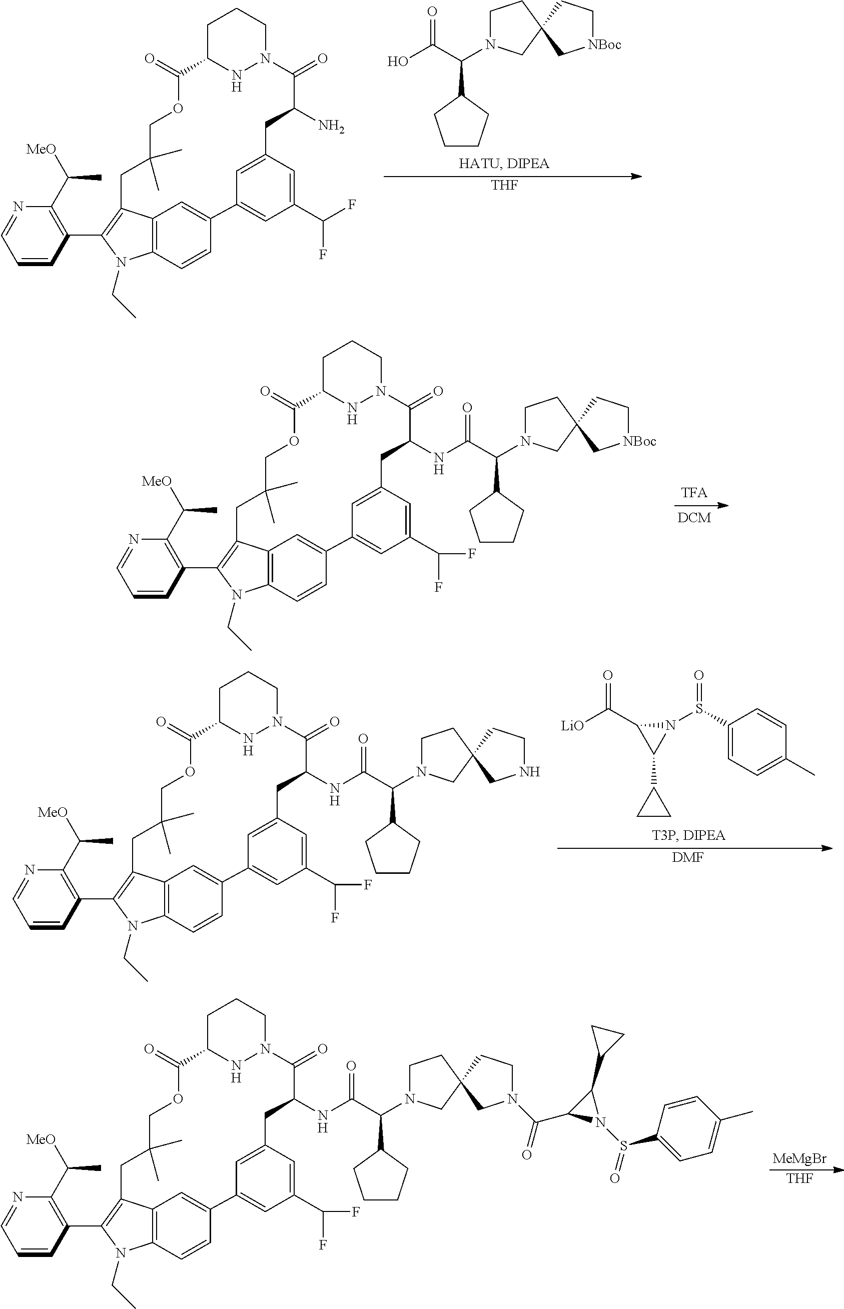

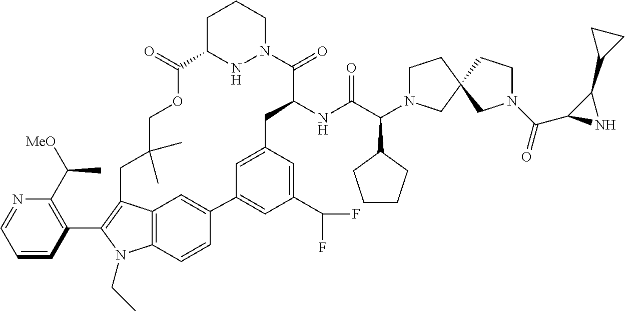

### Step 1: Synthesis of tert-butyl (5S)-7-((1 S)-1-cyclopentyl-2-(((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

To a solution of (S)-2-((S)-7-(tert-butoxycarbonyl)-2,7-diazaspiro[4.4]nonan-2-yl)-2-cyclopentylacetic acid (313.87 mg, 890.49 μmol) in THE (0.4 mL) was added HATU (338.59 mg, 890.49 μmol) and DIPEA (230.17 mg, 1.78 mmol) at 0° C. The reaction mixture was stirred for 30 min at room temperature, followed by the addition of a solution of (6³S,4S)-4-amino-2⁵-(difluoromethyl)-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (400 mg, 593.66 μmol) in THE (0.4 mL). The reaction mixture was stirred at room temperature for 1 h, and was then poured into sat. aq. NH₄Cl (20 mL). The aqueous phase was extracted with EtOAc (3×10 mL) and the combined organic phases were washed with brine (10 mL), dried with anhydrous Na₂SO₄, filtered and concentrated under reduced pressure to give the product (800 mg, crude) as solid which was used directly in next step. LCMS (ESI) m/z [M+H] calcd for C₅₇H₇₆F₂N₇O₇: 1008.6; found: 1008.7

### Step 2: Synthesis of (2S)-2-cyclopentyl-N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

To a solution of tert-butyl (5S)-7-((1 S)-1-cyclopentyl-2-(((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (800 mg, 595.10 μmol) in DCM (8 mL) was added TFA (1.36 g, 11.90 mmol) at 0° C., then the reaction was stirred at room temperature for 1 h. The reaction mixture was added dropwise to sat. aq. NaHCO₃ (30 mL) at 0° C. The mixture was extracted with DCM (3×10 mL) and the combined organic phases were washed with brine (10 mL), dried with anhydrous Na₂SO₄, filtered and concentrated under reduced pressure to give the product (630 mg, crude) as solid, which was used directly in the next step. LCMS (ESI) m/z [M+H] calcd for C₅₂H₆₈F₂N₇O₅: 908.5; found: 908.6.

### Step 3: Synthesis of (2S)-2-cyclopentyl-2-((S)-7-((2R,3R)-3-cyclopropyl-1-((R)-p-tolylsulfinyl) aziridine-2-carbonyl)-2,7-diazaspiro[4.4]nonan-2-yl)-N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)acetamide

To a solution of (2R,3R)-3-cyclopropyl-1-((R)-p-tolylsulfinyl)aziridine-2-carboxylic acid (350.60 mg, 1.32 mmol), (2S)-2-cyclopentyl-N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (600 mg, 660.70 μmol) and DIPEA (512.33 mg, 3.96 mmol) in DMF (6 mL) was added T3P (504.53 mg, 792.84 μmol) at 0° C., the reaction was then stirred at room temperature for 1 h. The residue was poured into sat. aq. NH₄Cl (60 mL). The aqueous phase was extracted with EtOAc (3×30 mL) and the combined organic phases were washed with brine (2×20 mL), dried with anhydrous Na₂SO₄, filtered and concentrated under reduced pressure. Purification by silica gel column chromatography (10→50% MeOH/DCM) afforded the product (560 mg, 57% yield) as solid. LCMS (ESI) m/z [M+H] calcd for C₆₅H₈₁F₂NaO₇S: 1155.6; found: 1155.7.

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)acetamide

To a solution of (2S)-2-cyclopentyl-2-((S)-7-((2R,3R)-3-cyclopropyl-1-((R)-p-tolylsulfinyl) aziridine-2-carbonyl)-2,7-diazaspiro[4.4]nonan-2-yl)-N-((6³S,4S)-2⁵-(difluoromethyl)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)acetamide (500 mg, 432.74 μmol) in THE (7.5 mL) was added MeMgBr (3 M, 1.15 mL) at −70° C. under N₂. The mixture was stirred at −70° C. for 1 h. The reaction mixture was quenched with sat. aq. NH₄Cl (30 mL). Then the aqueous phase was extracted with EtOAc (3×10 mL) and the combined organic layers were washed with brine (10 mL), dried with anhydrous Na₂SO₄, filtered and concentrated under reduced pressure. The crude product was purified by reverse phase chromatography (40→70% MeCN/H₂O 0.1% NH₄HCO₃) to give the product (105.94 mg, 24% yield) as solid. LCMS (ESI) m/z [M+H] calcd for C₅₈H₇₅F₂N₈O₆: 1017.58; found: 1017.4.

### Synthesis of (2S)-2-cyclopentyl-2-((S)-7-((2R,3R)-3-cyclopropylaziridine-2-carbonyl)-2,7-diazaspiro[4.4]nonan-2-yl)-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide

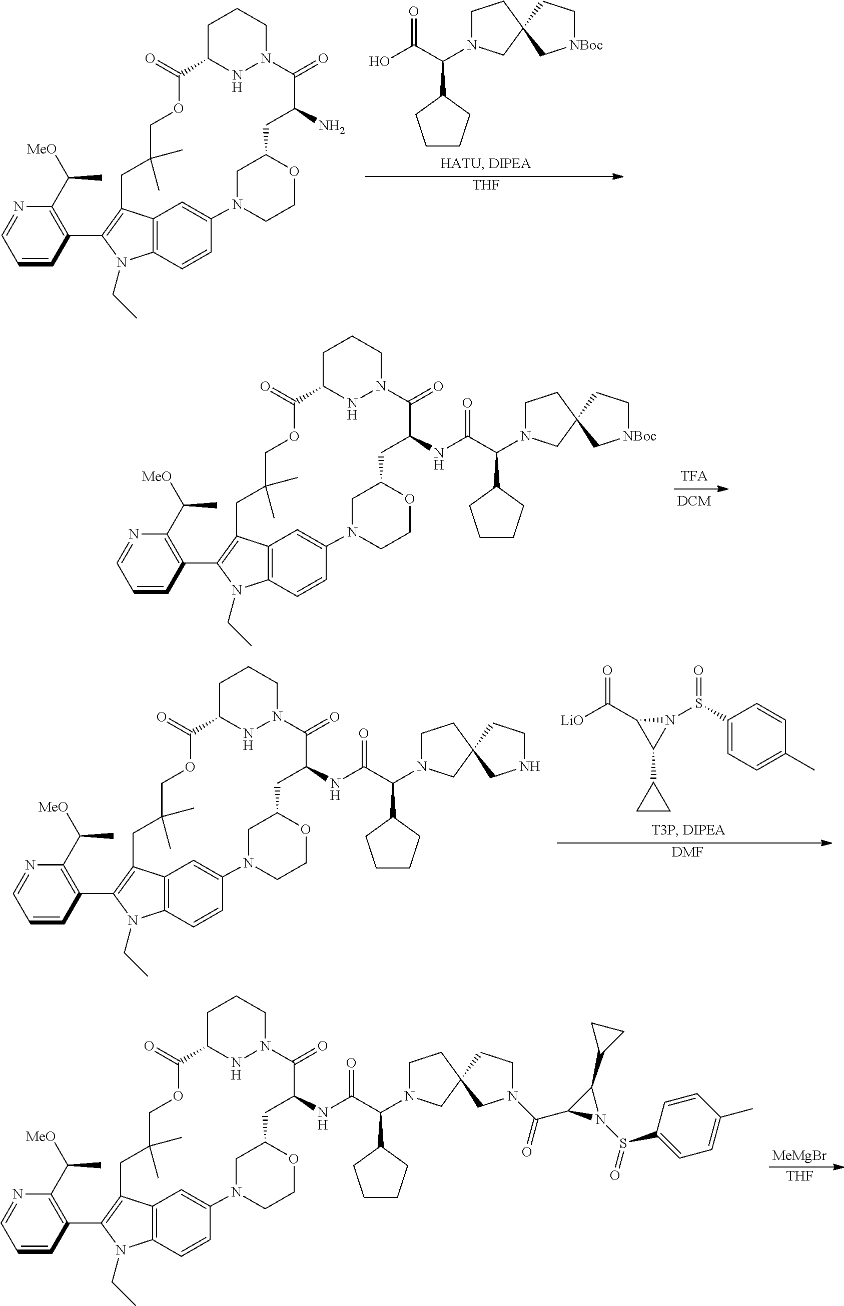

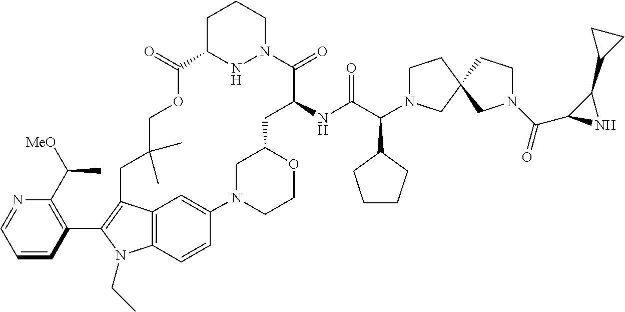

### Step 1: Synthesis of tert-butyl (5S)-7-((1S)-1-cyclopentyl-2-(((2²S,6³S,4S)-1-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-2-oxoethyl)-2,7-diazaspiro[4.4]nonane-2-carboxylate

To a solution of (S)-2-((S)-7-(tert-butoxycarbonyl)-2,7-diazaspiro [4.4]nonan-2-yl)-2-cyclopentylacetic acid (310 mg, 879.51 μmol) in MeCN (4 mL) was added HATU (334.42 mg, 879.51 μmol) and DIPEA (227.34 mg, 1.76 mmol), the mixture was stirred at room temperature for 30 min, followed by the addition of (2²S,6³S,4S)-4-amino-1¹-ethyl-1²-(2-((S)-1-methoxyethyl)pyridin-3-yl)-10,10-dimethyl-6¹,6²,6³,6⁴,6⁵,6⁶-hexahydro-1¹H-8-oxa-2(4,2)-morpholina-1 (5,3)-indola-6(1,3)-pyridazinacycloundecaphane-5,7-dione (371.03 mg, 586.34 μmol) at room temperature. The mixture was then stirred at room temperature for 1 h. The mixture was poured in H₂O (20 mL), extracted with EtOAc (3×20 mL), the combined organic layers were washed with brine (20 mL), dried with anhydrous Na₂SO₄, filtered, and concentrated under reduced pressure. The crude residue was purified by silica gel column chromatography (0→10% MeOH/EtOAc) to afford the product (400 mg, 71% yield) as a solid. LCMS (ESI) m/z [M+H] calcd for C₅₄H₇₉N₈O₈: 966.59; found: 967.7.

### Step 2: Synthesis of (2S)-2-cyclopentyl-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1 (5,3)-indola-6(1,3)-pyridazinacycloundecaphane-4-yl)-2-((S)-2,7-diazaspiro[4.4]nonan-2-yl)acetamide

To a solution of tert-butyl (5S)-7-((1 S)-1-cyclopentyl-2-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-2-oxoethyl)-2,7-diazaspiro[4.4]nonane-2-carboxylate (400 mg, 413.55 μmol) in DCM (1 mL) was added TFA (943.08 mg, 8.27 mmol) at 0° C., then the mixture was stirred at room temperature for 1 h. The mixture was poured into sat. aq. NaHCO₃ (30 mL), extracted with DCM (3×20 mL), then washed with brine (20 mL) and the organic phase was dried with anhydrous Na₂SO₄, filtered and concentrated under reduced pressure to afford the product (220 mg, 61% yield) as a solid. LCMS (ESI) m/z [M+H] calcd for C₄₉H₇₁N₈O₆: 866.54; found: 867.6.

### Step 3: Synthesis of (2S)-2-cyclopentyl-2-((S)-7-((2R,3R)-3-cyclopropyl-1-((R)-p-tolylsulfinyl)aziridine-2-carbonyl)-2,7-diazaspiro[4.4]nonan-2-yl)-N-((2²S,6³S,4S)-1¹-ethyl-1²-(2-isopropylpyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide

To a solution of (2S)-2-cyclopentyl-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-2-((S)-2,7-diazaspiro[4.4]nonan-2-yl)acetamide (320 mg, 369.03 μmol) and (2R,3R)-3-cyclopropyl-1-((R)-p-tolylsulfinyl)aziridine-2-carboxylic acid (209.82 mg, 553.55 μmol) in DMF (3.5 mL) was added T3P (258.32 mg, 405.94 μmol) and DIPEA (190.78 mg, 1.48 mmol) at room temperature, then the reaction was stirred for 1 h. The mixture was poured in H₂O (20 mL), extracted with EtOAc (2×20 mL), and washed with brine (20 mL). The organic phase was dried with anhydrous Na₂SO₄, filtered and concentrated under reduced pressure. The residue was purified by silica gel column chromatography (10% MeOH/DCM) to afford the product (230 mg, 56% yield) as a solid.

LCMS (ESI) m/z [M+H] calcd for C₆₂H₈₄N₉O₈S: 1113.61; found: 1114.7

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide

To a solution of (2S)-2-cyclopentyl-2-((S)-7-((2R,3R)-3-cyclopropyl-1-((R)-p-tolylsulfinyl)aziridine-2-carbonyl)-2,7-diazaspiro[4.4]nonan-2-yl)-N-((2²S,6³S,4S)-1¹-ethyl-1²-(2-isopropylpyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide (230 mg, 206.38 μmol) in THE (3 mL) was added MeMgBr (3 M, 550.35 μL) at −78° C., then the mixture was stirred at −78° C. for 1 h. The reaction mixture was quenched with sat. aq. NH₄Cl (20 mL). The aqueous phase was extracted with DCM (3×20 mL) and the combined organic layers were washed with brine (20 mL), dried with anhydrous Na₂SO₄, filtered and concentrated under reduced pressure. The crude was purified by reverse phase chromatography (35→60% MeCN/H₂O 0.1% NH₄HCO₃) to afford the product (70.34 mg, 35% yield) as a solid. LCMS (ESI) m/z [M+H] calcd for C₅₅H₇₈N₉O₇: 976.61; found: 976.6.

### Synthesis of (2S)-2-((R)-7-((2R,3R)-3-cyclopropylaziridine-2-carbonyl)-2,7-diazaspiro[4.4]nonan-2-yl)-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methylbutanamide

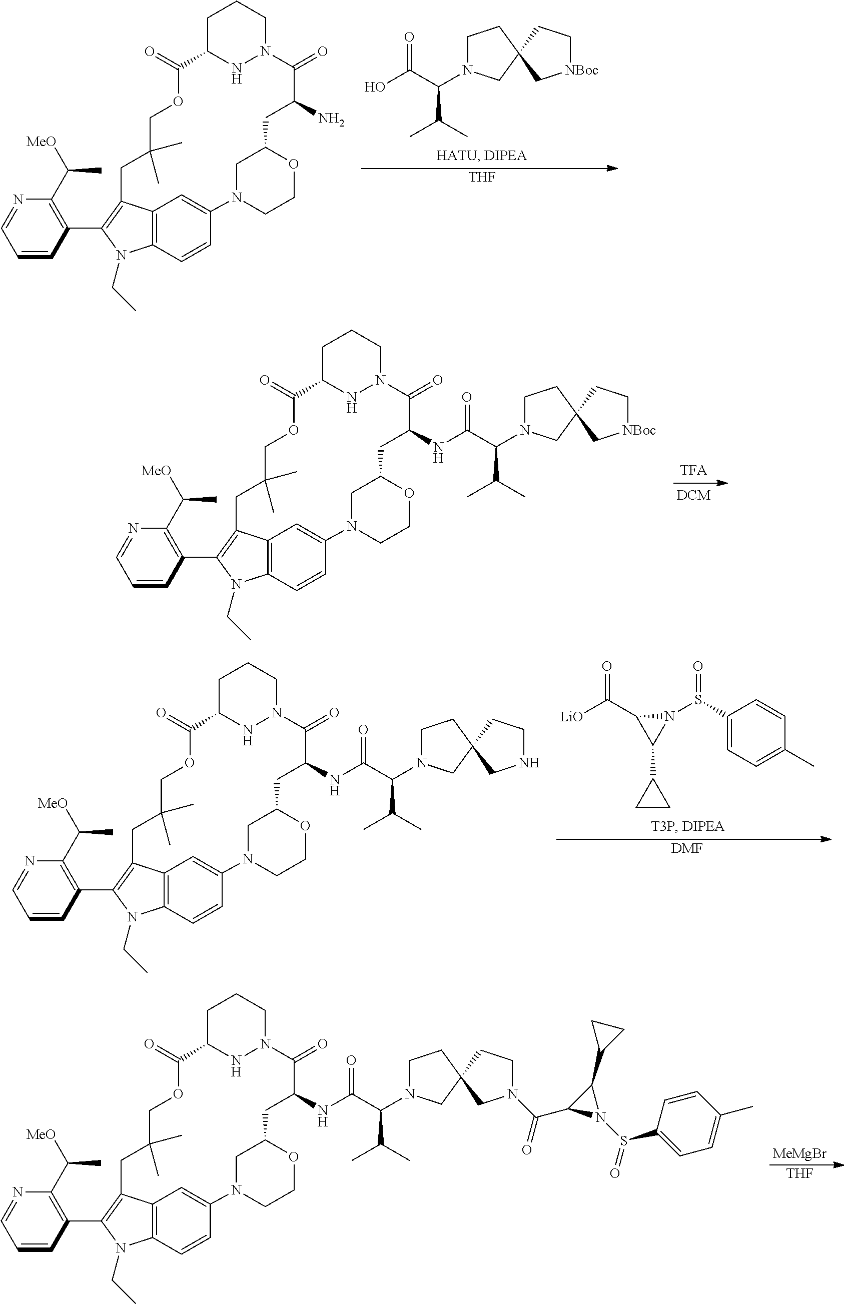

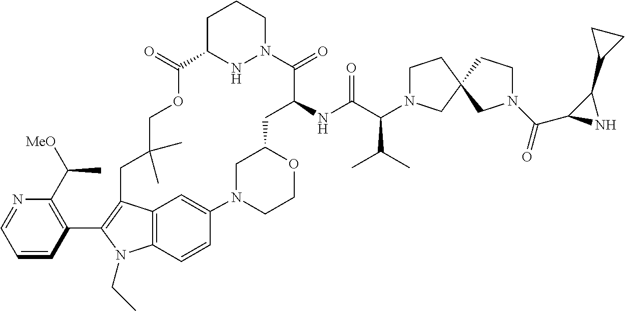

### Step 1: Synthesis of tert-butyl (5R)-7-((2S)-1-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1 (5,3)-indola-6(1,3)-pyridazinacycloundecaphane-4-yl)amino)-3-methyl-1-oxobutan-2-yl)-2,7-diazaspiro[4.4]nonane-2-carboxylate

To a solution of (S)-2-((R)-7-(tert-butoxycarbonyl)-2,7-diazaspiro[4.4]nonan-2-yl)-3-methylbutanoic acid (346.66 mg, 1.06 mmol) in THE (4 mL) was added DIPEA (257.35 mg, 1.99 mmol) and HATU (403.79 mg, 1.06 mmol) at 0° C., then the mixture was stirred for 30 min, followed by the addition of (2²S,6³S,4S)-4-amino-1¹-ethyl-1²-(2-((S)-1-methoxyethyl) pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (420 mg, 663.73 μmol). The resulting mixture was stirred at room temperature for 1 h. H₂O (20 mL) was added to the mixture and the mixture was extracted with EtOAc (3×15 mL) and the organic phase was washed with sat. aq. NH₄Cl (3×20 mL), dried with anhydrous Na₂SO₄, filtered and concentrated under reduced pressure to afford the product (780 mg, crude) as a solid. LCMS (ESI) m/z [M+H] calcd for C₅₂H₇₇N₈O₈: 941.58; found: 941.5

### Step 2: Synthesis of (2S)—N-((2²S,6³S,4S)-1¹-ethyl-1²-(2-((S)-1-methoxyethyl)pyridine-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methyl-2-((R)-2,7-diazaspiro[4.4]nonan-2-yl)butanamide

To a solution of tert-butyl (5R)-7-((2S)-1-(((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1 (5,3)-indola-6(1,3)-pyridazinacycloundecaphane-4-yl)amino)-3-methyl-1-oxobutan-2-yl)-2,7-diazaspiro[4.4]nonane-2-carboxylate (620 mg, 658.73 μmol) in DCM (6 mL) was added TFA (1.50 g, 13.17 mmol) at 0° C., then the mixture was stirred at room temperature for 1 h. The mixture was added dropwise to 100 mL cold sat. aq. NaHCO₃, then the mixture was extracted with DCM (3×50 mL). The combined organic phases were concentrated under reduced pressure to afford the product (520 mg, 73% yield) as a solid. LCMS (ESI) m/z [M+H] calcd for C₄₇H₆₉N₈O₆: 841.53; found: 841.4

### Step 3: Synthesis of (2S)-2-((R)-7-((2R,3R)-3-cyclopropyl-1-((R)-p-tolylsulfinyl)aziridine-2-carbonyl)-2,7-diazaspiro[4.4]nonan-2-yl)-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)- pyridazinacycloundecaphane-4-yl)-3-methylbutanamide

To a solution of (2S)—N-((2²S,6³S,4S)-1¹-ethyl-1²-(2-((S)-1-methoxyethyl)pyridine-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methyl-2-((R)-2,7-diazaspiro[4.4]nonan-2-yl)butanamide (420 mg, 499.35 μmol) in DMF (4 mL) was added (2R,3R)-3-cyclopropyl-1-((R)-p-tolylsulfinyl)aziridine-2-carboxylic acid (226.60 mg, 599.22 μmol, Li) at room temperature, followed by DIPEA (258.15 mg, 2.00 mmol) and T₃P (349.55 mg, 549.29 μmol). The mixture was stirred at room temperature for 1 h. H₂O (50 mL) was added to the mixture, the mixture was extracted with EtOAc (3×20 mL), and the organic phase was washed with sat. aq. NH₄Cl (3×20 mL), dried with anhydrous Na₂SO₄, filtered and concentrated under reduced pressure. The residue was purified by silica gel column chromatography (0→100% EtOAc/pet. ether, then 0→20% MeOH/EtOAc) to give the product (370 mg, 61% yield) as a solid. LCMS (ESI) m/z [M+H] calcd for C₆₀H₈₂N₉O₈S: 1088.59; found: 1088.7

### Step 4: Synthesis of (2S)-2-((R)-7-((2R,3R)-3-cyclopropylaziridine-2-carbonyl)-2,7-diazaspiro[4.4]nonan-2-yl)-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1 (5,3)-indola-6(1,3)-pyridazinacycloundecaphane- 4-yl)-3-methylbutanamide

To a solution of (2S)-2-((R)-7-((2R,3R)-3-cyclopropyl-1-((R)-p-tolylsulfinyl)aziridine-2-carbonyl)-2,7-diazaspiro[4.4]nonan-2-yl)-N-((2²S,6³S,4S)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-3-methylbutanamide (440 mg, 404.26 μmol) in THE (4.5 mL) was added MeMgBr (3 M, 1.08 mL) at −78° C., then the mixture was stirred at −78° C. for 1 h under N₂. The mixture was added dropwise to cold sat. aq. NH₄Cl (40 mL), then the mixture was extracted with DCM (3×20 mL). The combined organic phases were concentrated under reduced pressure. The crude was purified by reverse phase chromatography (35→65% MeCN/H₂O 0.1% NH₄HCO₃) to give the product (109.2 mg, 28% yield) as a solid. LCMS (ESI) m/z [M+H] calcd for C₅₃H₇₆N₉O₇: 950.59; found: 950.6

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

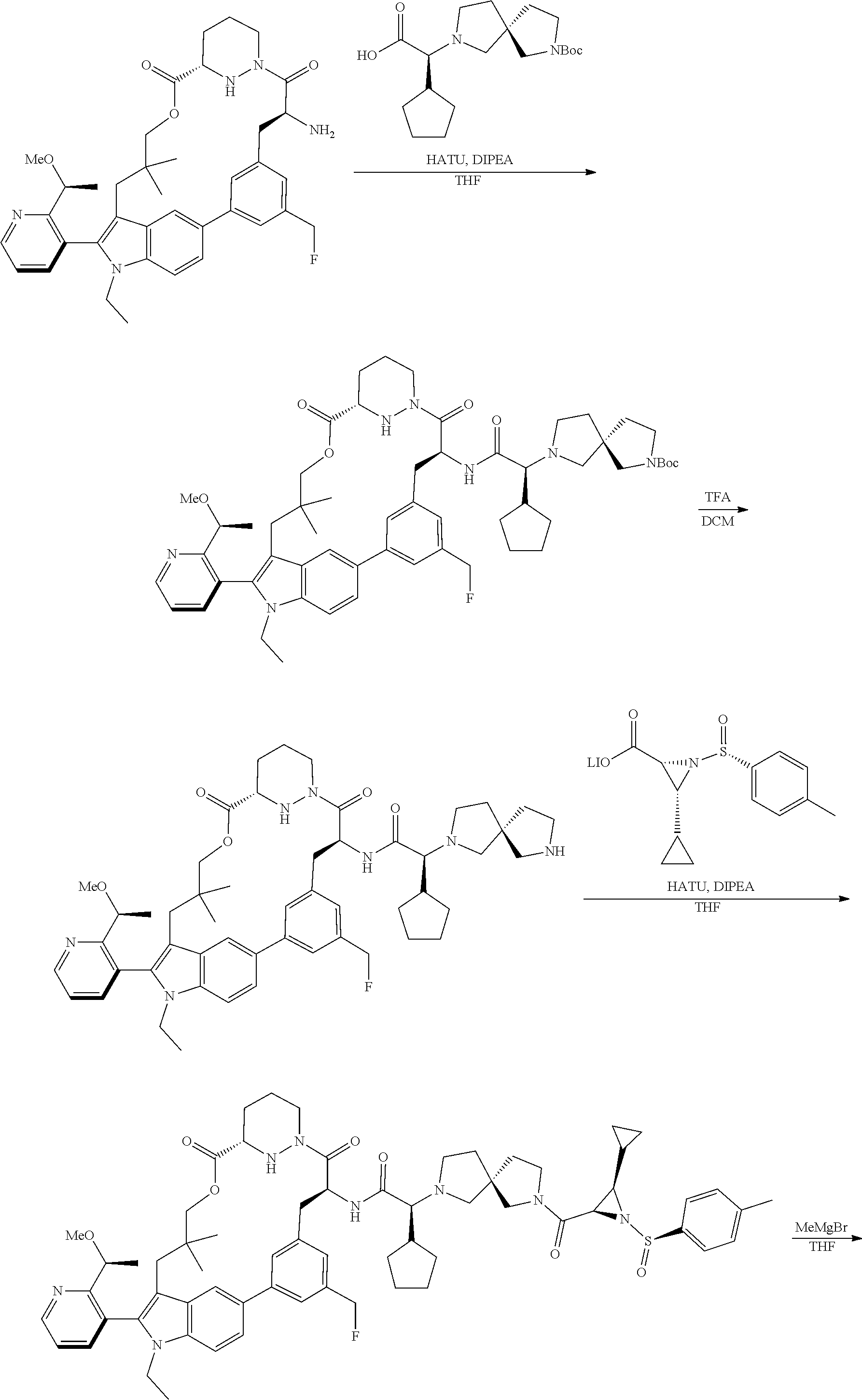

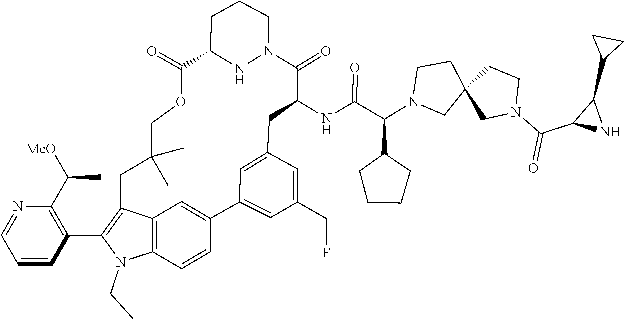

### Step 1: Synthesis of tert-butyl (5S)-7-((1 S)-1-cyclopentyl-2-(((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

To a solution of (S)-2-((S)-7-(tert-butoxycarbonyl)-2,7-diazaspiro[4.4]nonan-2-yl)-2-cyclopentylacetic acid (362.79 mg, 1.03 mmol) in THE (4.5 mL) was added HATU (391.36 mg, 1.03 mmol) and DIPEA (266.05 mg, 2.06 mmol) at 0° C. The reaction mixture was stirred for 30 min at room temperature, then a solution of (6³S,4S)-4-amino-1¹-ethyl-2⁵-(fluoromethyl)-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (450 mg, 686.19 μmol) in THE (4.5 mL) was added, and the reaction was stirred at room temperature for 1 h. The residue was poured into sat. aq. NH₄Cl (30 mL). The aqueous phase was extracted with EtOAc (3×20 mL) and the combined organic phases were washed with brine (10 mL), dried with anhydrous Na₂SO₄, filtered and concentrated under reduced pressure to give the product (900 mg, crude) as a solid, which was used directly in the next step.

LCMS (ESI) m/z [M+H] calcd for C₅₇H₇₇FN₇O₇: 990.6; found: 990.6.

### Step 2: Synthesis of tert-butyl (5S)-7-((1 S)-1-cyclopentyl-2-(((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

To a solution of tert-butyl (5S)-7-((1 S)-1-cyclopentyl-2-(((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (900 mg, 636.20 μmol) in DCM (9 mL) was added TFA (1.45 g, 12.72 mmol) at 0° C., then the reaction was stirred at room temperature for 1 h. The reaction mixture was added dropwise to sat. aq. NaHCO₃ (100 mL) at 0° C. The mixture was extracted with DCM (3×30 mL) and the combined organic phases were washed with brine (30 mL), dried with anhydrous Na₂SO₄, filtered and concentrated under reduced pressure to give the product (700 mg, 88% yield) as a solid. LCMS (ESI) m/z [M+H] calcd for C₅₂H₆₉FN₇O₅: 890.5; found: 890.7.

### Step 3: Synthesis of (2S)-2-cyclopentyl-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2R,3R)-3-cyclopropyl-1-((R)-p-tolylsulfinyl)aziridine-2-carboxylic acid (320.11 mg, 1.18 mmol, Li) in THE (6 mL) was added HATU (406.40 mg, 1.07 mmol) and DIPEA (207.21 mg, 1.60 mmol) at 0° C., and the mixture was stirred at room temperature for 30 min. A solution of tert-butyl (5S)-7-((1 S)-1-cyclopentyl-2-(((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (670 mg, 534.41 μmol) in THE (6 mL) was added and the reaction was stirred at room temperature for 1 h. The residue was poured into sat. aq. NH₄Cl (30 mL). The aqueous phase was extracted with EtOAc (3×15 mL) and the combined organic phases were washed with brine (10 mL), dried with anhydrous Na₂SO₄, filtered and concentrated under reduced pressure to give the crude product which was purified by silica gel column chromatography (0→100% EtOAc/pet. ether then 50% MeOH/EtOAc) to give to give (330 mg, 52% yield) as a solid. LCMS (ESI) m/z [M+H] calcd for C₆₅H₈₂FN₈O₇: 1137.6; found: 1137.7.

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)acetamide

To a solution of (2S)-2-cyclopentyl-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (400 mg, 351.66 μmol) in THE (6 mL) was added MeMgBr (3 M, 937.77 μL) at −70° C. under N₂. Then the mixture was stirred at −70° C. for 1 h. The reaction mixture was quenched with sat. aq. NH₄Cl (30 mL). The aqueous phase was extracted with EtOAc (3×10 mL) and the combined organic layers were washed with brine (10 mL), dried with anhydrous Na₂SO₄, filtered and concentrated under reduced pressure to give the crude product. The crude product was purified by reverse phase chromatography (40→70% MeCN/H₂O 0.1% NH₄HCO₃) to give the product (72 mg, 20% yield) as solid. LCMS (ESI) m/z [M+H] calcd for C₅₈H₇₆FN₈O₆: 999.59; found: 999.5

### Synthesis of (2S)-2-((R)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

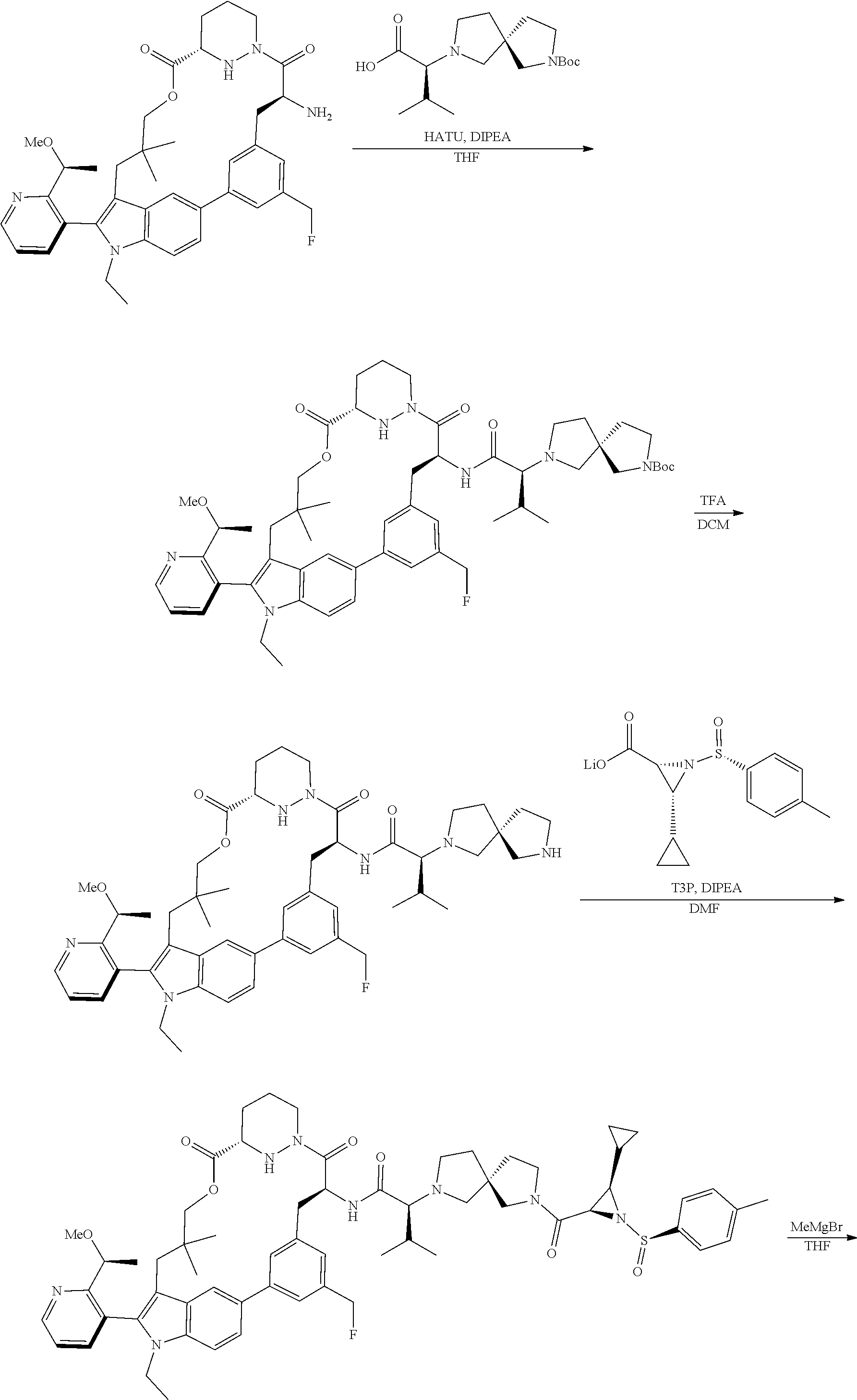

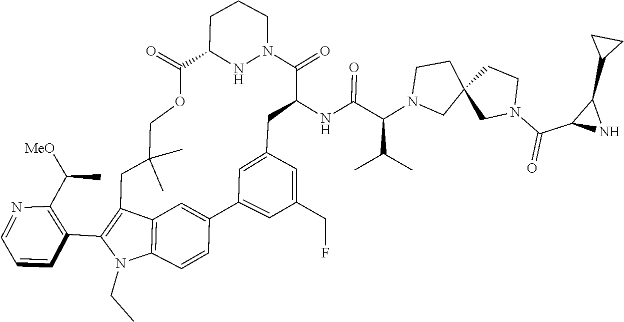

### Step 1: Synthesis of tert-butyl (5S)-7-((2S)-1-(((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-3-methyl-1-oxobutan-2-yl)-2,7-diazaspiro[4.4]nonane-2-carboxylate

To a solution of (S)-2-((S)-7-(tert-butoxycarbonyl)-2,7-diazaspiro[4.4]nonan-2-yl)-3-methylbutanoic acid (440 mg, 1.35 mmol) and HATU (580.85 mg, 1.53 mmol) in THE (5 mL) was added (6³S,4S)-4-amino-1¹-ethyl-2⁵-(fluoromethyl)-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (589.31 mg, 898.61 μmol) and DIPEA (290.35 mg, 2.25 mmol) at 0° C., then the mixture was stirred at room temperature for 1 h. The mixture was added into cold sat. aq. NH₄Cl (20 mL). Then the aqueous phase was extracted with EtOAc (3×20 mL). The combined organic layers were washed with brine (50 mL), dried with anhydrous Na₂SO₄, filtered and concentrated under reduced pressure to give the product (1.27 g, crude) as a solid. LCMS (ESI) m/z [M+H] calcd for C₅₅H₇₅FN₇O₇: 964.56; found: 964.7.

### Step 2: Synthesis of (2S)—N-((6³S,4S)-1¹-ethyl-2⁵-(fluoromethyl)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)-3-methyl-2-((S)-2,7-diazaspiro[4.4]nonan-2-yl)butanamide

To a solution of tert-butyl (5S)-7-((2S)-1-(((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-2,7-diazaspiro[4.4]nonane-2-carboxylate (1.27 g, 1.32 mmol) in DCM (12 mL) was added TFA (4.51 g, 39.51 mmol) at 0° C., then the mixture was stirred at room temperature for 1 h. The reaction mixture was added into cold sat. aq. NaHCO₃ (100 mL). Then the aqueous phase was extracted with EtOAc (3×50 mL) and the combined organic layers were washed with brine (50 mL), dried with anhydrous Na₂SO₄, filtered and concentrated under reduced pressure to give the product (890 mg, crude) as a solid. LCMS (ESI) m/z [M+H] calcd for C₅₀H₆₇FN₇O₅: 864.51; found: 864.6.

### Step 3: Synthesis of (2S)-2-((S)-7-((2R,3R)-3-cyclopropyl-1-((R)-p-tolylsulfinyl) aziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)-3-methylbutanamide

To a solution of (2R,3R)-3-cyclopropyl-1-((R)-p-tolylsulfinyl)aziridine-2-carboxylic acid (397.01 mg, 1.46 mmol, Li) and (2S)—N-((6³S,4S)-1¹-ethyl-2⁵-(fluoromethyl)-1²-(2-((S)-1-methoxyethyl) pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-((S)-2,7-diazaspiro[4.4]nonan-2-yl)butanamide (840 mg, 972.11 μmol) in DMF (9 mL) was added DIPEA (628.19 mg, 4.86 mmol) and T3P (309.31 mg, 972.11 μmol) at 0° C., the mixture was stirred at room temperature for 1 h. The reaction mixture was added into cold sat. aq. NH₄Cl (30 mL). Then the aqueous phase was extracted with EtOAc (3×20 mL) and the combined organic layers were washed with brine (50 mL), dried with anhydrous Na₂SO₄, filtered and concentrated under reduced pressure. The crude product was purified by silica gel column chromatography (0→10% MeOH/EtOAc) to afford the product (700 mg, 65% yield) as a solid. LCMS (ESI) m/z [M+H] calcd for C₆₃H₈₀FN₈O₇S: 1111.58; found: 1111.8.

### Step 4: Synthesis of (2S)-2-((S)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methylbutanamide

To a solution of (2S)-2-((S)-7-((2R,3R)-3-cyclopropyl-1-((R)-p-tolylsulfinyl) aziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methylbutanamide (350 mg, 314.92 μmol) in THE (4 mL) was added MeMgBr (3 M, 839.77 μL) at −78° C., then the mixture was stirred at −78° C. for 1 h. The reaction mixture was added into cold sat. aq. NH₄Cl (20 mL). Then the aqueous phase was extracted with EtOAc (3×20 mL) and the combined organic layers were washed with brine (20 mL), dried with anhydrous Na₂SO₄, filtered and concentrated under reduced pressure. The product was purified by reverse phase chromatography (45→65% MeCN/H₂O 0.1% NH₄HCO₃), to give the product (107.96 mg, 18% yield) as a solid. LCMS: (ESI) m/z [M+H] calcd for C₅₆H₇₄FN₈O₆: 973.57; found: 973.5.

### Synthesis of (6³S,4S)-4-((S)-2-((R)-7-((2R,3R)-3-cyclopropylaziridine-2-carbonyl)-1-oxo-2,7-diazaspiro[4.4]nonan-2-yl)-3-methylbutanamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl pivalate

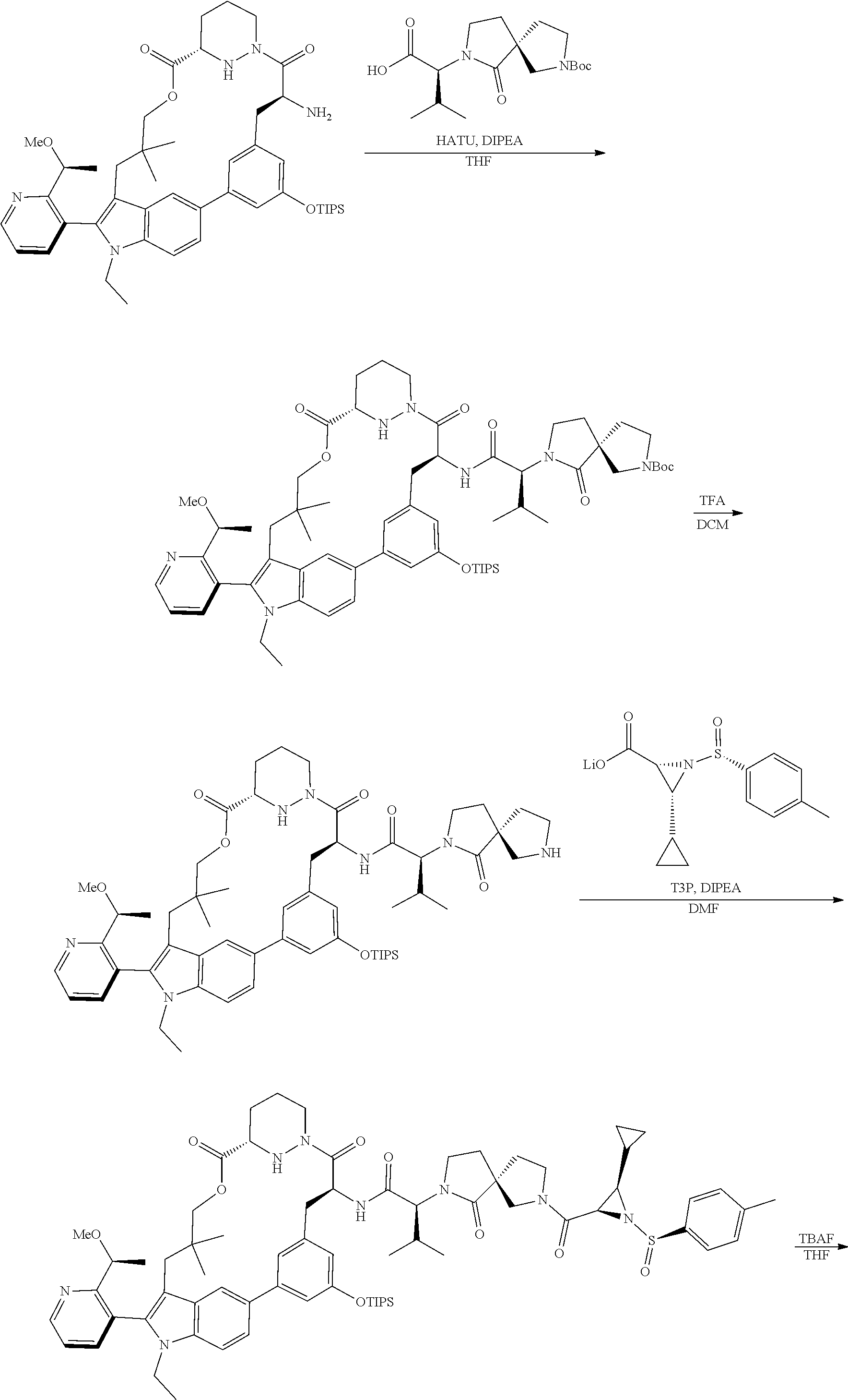

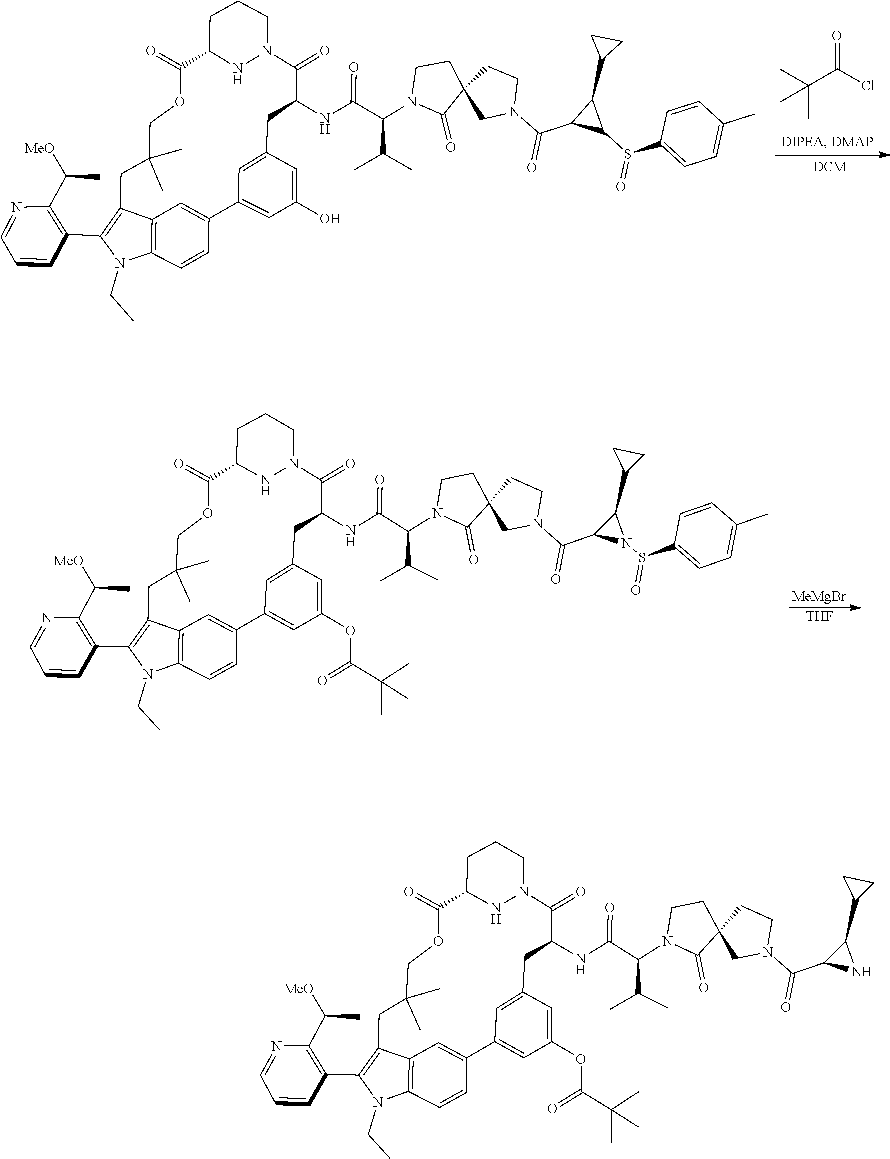

### Step 1: Synthesis of tert-butyl (5R)-7-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-6-oxo-2,7-diazaspiro[4.4]nonane-2-carboxylate

To a solution of (6³S,4S)-4-amino-1¹-ethyl-1²-(2-((S)-1-methoxyethyl)pyridin-3-yl)-10,10-dimethyl-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)- benzenacycloundecaphane-5,7-dione (0.55 g, 690.85 μmol) in THE (5.5 mL) was added (S)-2-((R)-7-(tert-butoxycarbonyl)-1-oxo-2,7-diazaspiro[4.4]nonan-2-yl)-3-methylbutanoic acid (246.93 mg, 725.39 μmol), DIPEA (446.44 mg, 3.45 mmol) and HATU (315.22 mg, 829.02 μmol), then the mixture was stirred at room temperature for 1 h. The reaction mixture was poured into cold H₂O (50 mL) and stirred for 5 min. The aqueous phase was extracted with EtOAc (3×20 mL) and the combined organic phase was washed with brine (20 mL), dried with anhydrous Na₂SO₄, filtered and concentrated under reduced pressure. The residue was purified by silica gel column chromatography (0→100% EtOAc/pet. ether) to give the product (0.82 g, crude) as a solid. LCMS (ESI) m/z [M+H] calcd for C₆₃H₉₂N₇O₉Si: 1118.66; found: 1118.7.

### Step 2: Synthesis of (2S)—N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-((S)-1-oxo-2,7-diazaspiro[4.4]nonan-2-yl)butanamide

To a solution of tert-butyl (5R)-7-((2S)-1-(((6³S,4S)-1¹-ethyl-1²-(2-((S)-1-methoxyethyl) pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-6-oxo-2,7-diazaspiro[4.4]nonane-2-carboxylate (0.89 g, 795.69 μmol) in DCM (9 mL) was added TFA (2.27 g, 19.89 mmol) at 0° C., then the mixture was stirred at room temperature under N₂ for 1 h. The mixture was dropped into sat. aq. NaHCO₃ (60 mL) at 0° C. and neutralized to pH 7˜8. The resulting mixture was extracted with DCM (2×20 mL) and the combined organic layers were washed with brine (30 mL), dried over Na₂SO₄, filtered and concentrated under reduced pressure to give the product (0.7 g, crude) as a solid. LCMS (ESI) m/z [M+H] calcd for C₅₈H₈₄N₇O₇Si: 1018.61; found: 1018.6.

### Step 3: Synthesis of (2S)-2-((S)-7-((2R,3R)-3-cyclopropyl-1-((R)-p-tolylsulfinyl)aziridine-2-carbonyl)-1-oxo-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a solution of (2S)—N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-((S)-1-oxo-2,7-diazaspiro[4.4]nonan-2-yl)butanamide (0.7 g, 687.35 μmol) and (2R,3R)-3-cyclopropyl-1-((R)-p-tolylsulfinyl)aziridine-2-carboxylic acid (246.20 mg, 904.27 μmol, Li) in DMF (7 mL) was added DIPEA (533.00 mg, 4.12 mmol) and T3P (568.62 mg, 893.55 μmol), then the mixture was stirred at room temperature for 1 h. The reaction mixture was poured into cold H₂O (80 mL) and stirred for 5 min. The aqueous phase was extracted with EtOAc (3×30 mL) and the combined organic phases were washed with brine (20 mL), dried with anhydrous Na₂SO₄, filtered and concentrated under reduced pressure. The residue was purified by silica gel column chromatography (0→100% EtOAc/pet. ether) to give the product (0.7 g, 81% yield) as a solid.

### Step 4: Synthesis of (2S)-2-((S)-7-((2R,3R)-3-cyclopropyl-1-((R)-p-tolylsulfinyl)aziridine-2-carbonyl)-1-oxo-2,7-diazaspiro[4.4]nonan-2-yl)-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)-3-methylbutanamide

To a solution of (2S)-2-((S)-7-((2R,3R)-3-cyclopropyl-1-((R)-p-tolylsulfinyl)aziridine-2-carbonyl)-1-oxo-2,7-diazaspiro[4.4]nonan-2-yl)-N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (0.7 g, 553.05 μmol) in THE (7 mL) was added TBAF (1 M, 1.66 mL), and the mixture was stirred at room temperature under N₂ for 1 h. The reaction was quenched with sat. aq. NH₄Cl/H₂O (1:1, 70 mL) and extracted with EtOAc (3×20 mL). The combined organic phases were washed with brine (30 mL), dried with anhydrous Na₂SO₄, filtered and concentrated under reduced pressure. The residue was purified by silica gel column chromatography (0→100% EtOAc/pet. ether) to give the product (0.54 g, 88% yield) as a solid.

### Step 5: Synthesis of (6³S,4S)-4-((S)-2-((S)-7-((2R,3R)-3-cyclopropyl-1-((R)-p-tolylsulfinyl) aziridine-2-carbonyl)-1-oxo-2,7-diazaspiro[4.4]nonan-2-yl)-3-methylbutanamido)-1′-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl pivalate

To a solution of (2S)-2-((S)-7-((2R,3R)-3-cyclopropyl-1-((R)-p-tolylsulfinyl)aziridine-2-carbonyl)-1-oxo-2,7-diazaspiro[4.4]nonan-2-yl)-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methylbutanamide (0.54 g, 486.76 μmol) and DIPEA (188.73 mg, 1.46 mmol) in DCM (8 mL) was added 2,2-dimethylpropanoyl chloride (76.30 mg, 632.79 μmol) and DMAP (5.95 mg, 48.68 μmol) at 0° C., the mixture was stirred at room temperature under N₂ for 1 h. The reaction mixture was poured into cold H₂O (100 mL) and extracted with DCM (3×30 mL). The combined organic layers were washed with brine (40 mL), dried over Na₂SO₄, filtered and concentrated under reduced pressure to give a residue. The crude product was purified by silica gel column chromatography (0→100% EtOAc/pet. ether) to give the product (0.51 g, 88% yield) as a solid.

### Step 6: Synthesis of (6³S,4S)-4-((S)-2-((S)-7-((2R,3R)-3-cyclopropylaziridine-2-carbonyl)-1-oxo-2,7-diazaspiro[4.4]nonan-2-yl)-3-methylbutanamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-25-yl pivalate

To a solution of (6³S,4S)-4-((S)-2-((S)-7-((2R,3R)-3-cyclopropyl-1-((R)-p-tolylsulfinyl) aziridine-2-carbonyl)-1-oxo-2,7-diazaspiro[4.4]nonan-2-yl)-3-methylbutanamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl pivalate (0.49 g, 410.56 μmol) in THE (5 mL) was added MeMgBr (3 M, 615.84 μL) at −78° C., then the mixture was stirred at −78° C. under N₂ for 30 min. The reaction mixture was quenched with sat. aq. NH₄Cl (60 mL) and then the aqueous phase was extracted with EtOAc (3×20 mL). The combined organic layers were washed with brine (20 mL), dried with anhydrous Na₂SO₄, filtered and concentrated under reduced pressure. The residue was purified by reverse phase chromatography (45→75% MeCN/H₂O 0.1% NH₄HCO₃) to give the product (232.42 mg, 53% yield) as a solid. LCMS (ESI) m/z [M+H] calcd for C₆₀H₇₉N₈O₉: 1055.60; found: 1055.5.

### Synthesis of (6³S,4S)-4-((S)-2-cyclopentyl-2-((S)-7-((2R,3R)-3-cyclopropylaziridine-2-carbonyl)-1-oxo-2,7-diazaspiro[4.4]nonan-2-yl)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-ylisobutyrate

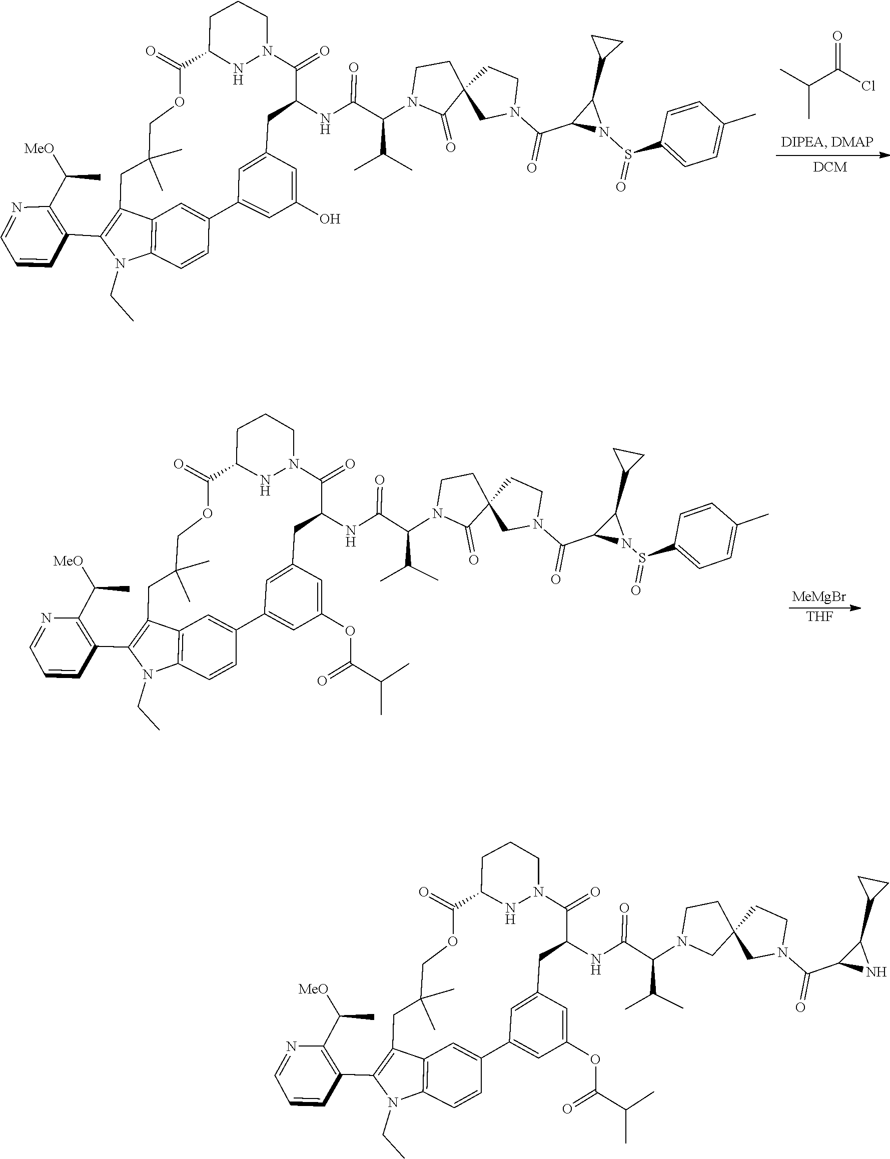

### Step 1: Synthesis of (6³S,4S)-4-((S)-2-cyclopentyl-2-((S)-7-((2R,3R)-3-cyclopropyl-1-((R)-p-tolylsulfinyl)aziridine-2-carbonyl)-1-oxo-2,7-diazaspiro[4.4]nonan-2-yl)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl isobutyrate

To a solution of (2S)-2-cyclopentyl-2-((S)-7-((2R,3R)-3-cyclopropyl-1-((R)-p-tolylsulfinyl) aziridine-2-carbonyl)-1-oxo-2,7-diazaspiro[4.4]nonan-2-yl)-N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (750 mg, 660.55 μmol) and DIPEA (256.12 mg, 1.98 mmol) in DCM (8 mL) was added 2-methylpropanoyl chloride (91.50 mg, 858.72 μmol) and DMAP (8.07 mg, 66.06 μmol) at 00C. The mixture was stirred at room temperature for 1 h. The mixture was added into sat. aq. NH₄Cl (20 mL) and then the mixture was extracted with EtOAc (3×10 mL) and separated. The combined organic layers were washed with brine (20 mL), dried with anhydrous Na₂SO₄, filtered and concentrated under reduced pressure to give the crude product. The product was purified by silica gel column chromatography (0→10% MeOH/EtOAc) to give the product (500 mg, 63% yield) as solid. LCMS: (ESI) m/z [M+H] calcd for C₆₈H₈₅N₈O₁₀S:1025.60; found: 1025.6.

### Step 2: Synthesis of (6³S,4S)-4-((S)-2-cyclopentyl-2-((S)-7-((2R,3R)-3-cyclopropylaziridine-2-carbonyl)-1-oxo-2,7-diazaspiro[4.4]nonan-2-yl)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-25-yl isobutyrate

To a solution of (6³S,4S)-4-((S)-2-cyclopentyl-2-((S)-7-((2R,3R)-3-cyclopropyl-1-((R)-p-tolylsulfinyl)aziridine-2-carbonyl)-1-oxo-2,7-diazaspiro[4.4]nonan-2-yl)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-25-yl isobutyrate (400 mg, 331.81 μmol) in THE (5 mL) was added MeMgBr (3 M, 884.83 μL) at −78° C., and stirred for 1 h. The mixture was added into sat. aq. NH₄Cl (20 mL). The mixture was extracted with EtOAc (3×10 mL) and the combined organic layers were washed with brine (20 mL), dried with anhydrous Na₂SO₄, filtered and concentrated under reduced pressure to give the crude product. The crude product was purified by reverse phase chromatography (45→65% MeCN/H₂O 0.1% NH₄HCO₃) and the eluent was lyophilized to give the product (103.15 mg, 29% yield) as a solid. LCMS: (ESI) m/z [M+H] calcd for C₆₁H₇₉N₈O₉:1067.60; found: 1067.6.

### Synthesis of (6³S,4S)-4-((S)-2-cyclopentyl-2-(2-((2R,3R)-3-cyclopropyl-N-methylaziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl dihydrogen phosphate

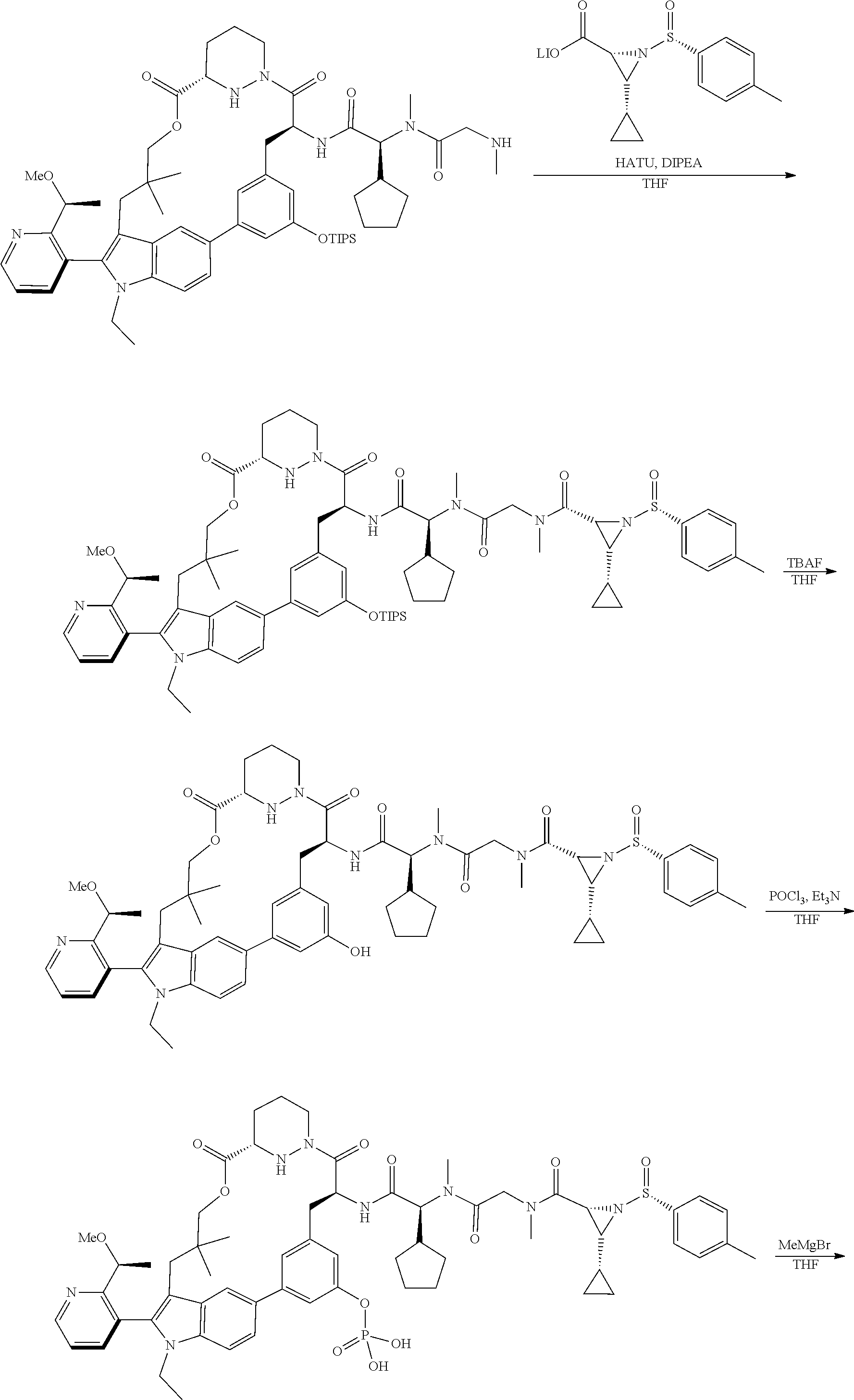

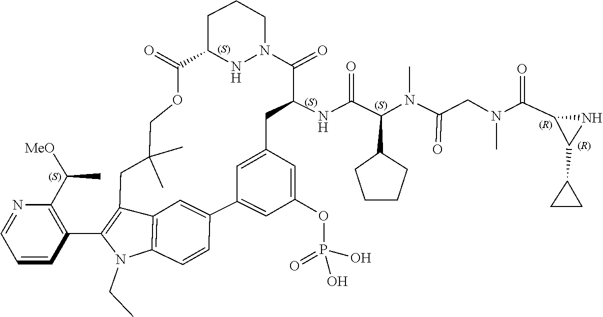

### Step 1: Synthesis of (2R,3R)—N-(2-(((1 S)-1-cyclopentyl-2-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsulfinyl)aziridine-2-carboxamide

To a solution of (2S)-2-cyclopentyl-N-((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(N-methyl-2-(methylamino)acetamido)acetamide (0.2 g, 198.73 μmol) and (2R,3R)-3-cyclopropyl-1-((R)-p-tolylsulfinyl)aziridine-2-carboxylic acid (113.91 mg, 317.97 μmol, Li) in THE (3.5 mL) was added DIPEA (115.58 mg, 894.28 μmol) and HATU (113.34 mg, 298.09 μmol) at 0° C. The mixture was stirred at room temperature for 3 h. The reaction mixture was poured into cold H₂O (30 mL) and extracted with EtOAc (3×15 mL). The combined organic layers were washed with brine (10 mL), dried over Na₂SO₄, filtered and concentrated under reduced pressure to give a residue. The crude product was purified by silica gel column chromatography (0→100% EtOAc/pet. ether) to give the product (0.2 g, 80% yield) as a solid.

LCMS (ESI) m/z [M+H] calcd for C₇₀H₉₇N₈O₉SSi: 1253.7; found: 1253.7.

### Step 2: Synthesis of (2R,3R)—N-(2-(((1 S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsulfinyl)aziridine-2-carboxamide

To a solution of (2R,3R)—N-(2-(((1 S)-1-cyclopentyl-2-(((6³S,4S)-1¹-ethyl-1²-(2-((S)-1-methoxyethyl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsulfinyl)aziridine-2-carboxamide (0.2 g, 159.53 μmol) in THE (1.5 mL) was added TBAF (1 M, 191.43 μL) at 0° C. The mixture was stirred at room temperature for 1 h. The reaction was quenched with sat. aq. NH₄Cl/H₂O (1:1, 20 mL) and was extracted with EtOAc (10×3 mL). The combined organic layers were washed with brine (10 mL), dried with Na₂SO₄, filtered and concentrated under reduced pressure. The crude product was purified by silica gel column chromatography (0→100% EtOAc/pet. ether) to give the product (0.14 g, 80% yield) as a solid. LCMS (ESI) m/z [M+H] calcd for C₆₁H₇₆N₈O₉S: 1097.5; found: 1097.5.

### Step 3: Synthesis of (6³S,4S)-4-((S)-2-cyclopentyl-2-(2-((2R,3R)-3-cyclopropyl-N-methyl-1-((R)-p-tolylsulfinyl)aziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl dihydrogen phosphate

To a solution of (2R,3R)—N-(2-(((1 S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsulfinyl)aziridine-2-carboxamide (1 g, 911.27 μmol) in THE (50 mL) at 0° C. was added Et₃N (1.90 mL, 13.67 mmol), followed by POCl₃ (127.02 μL, 1.37 mmol), then the mixture was stirred for 30 min at 0° C. Na₂CO₃ (20 mL) was added and the resulting mixture was stirred for 4 h at room temperature. The mixture was adjusted to pH 7 with 1 N HCl and was then extracted with DCM (5×30 mL). The combined organic layers were concentrated under reduced pressure to give the product (1.07 g, crude) as a solid, which was used directly in the next step. LCMS (ESI) m/z [M+H] calcd for C₆₁H₇₈NaO₁₂PS: 1177.51; found 1177.6.

### Step 4: Synthesis of (6³S,4S)-4-((S)-2-cyclopentyl-2-(2-((2R,3R)-3-cyclopropyl-N-methylaziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl dihydrogen phosphate

To a solution of (6³S,4S)-4-((S)-2-cyclopentyl-2-(2-((2R,3R)-3-cyclopropyl-N-methyl-1-((R)-p-tolylsulfinyl)aziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl dihydrogen phosphate (400 mg, 339.75 μmol) in THE (4 mL) was added MeMgBr (3 M, 905.99 μL) at −78° C., then the mixture was stirred at −78° C. for 1 h. The reaction mixture was quenched with sat. aq. NH₄Cl (20 mL), then the aqueous phase was extracted with DCM (3×20 mL). The combined organic layers were washed with brine (20 mL), dried with anhydrous Na₂SO₄, filtered and concentrated under reduced pressure. The crude product was purified by reverse phase chromatography (25→55% MeCN/H₂O 0.1% NH₄HCO₃) to afford the product (31.60 mg, 8% yield) as a solid. LCMS (ESI) m/z [M+H] calcd for C₅₄H₇₂N₈O₁₁P: 1039.51; found: 1039.5.

### Synthesis of (2R,3R)—N-(2-(((1S)-2-(((6³S,4S)-2⁵-((tert-butyldimethylsilyl)oxy)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-1-cyclopentyl-2-oxoethyl)(methyl)amino)-2-oxoethyl)-3-cyclopropyl-N-methylaziridine-2-carboxamide

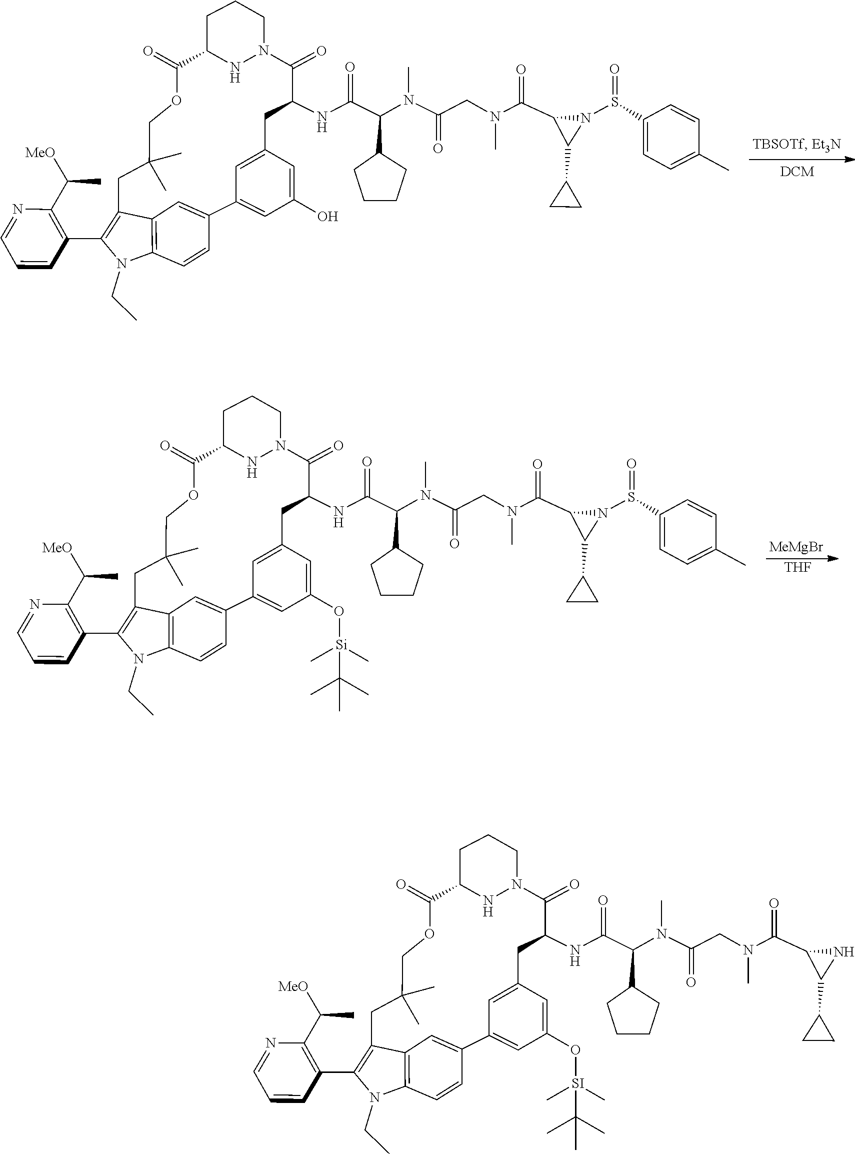

### Step 1: Synthesis of (2R,3R)—N-(2-(((1 S)-2-(((6³S,4S)-2⁵-((tert-butyldimethylsilyl)oxy)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-1-cyclopentyl-2-oxoethyl)(methyl)amino)-2-oxoethyl)-3-cyclopropyl-N-methyl-1-((R)-p-tolylsulfinyl)aziridine-2-carboxamide

To a solution of (2R,3R)—N-(2-(((1 S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsulfinyl)aziridine-2-carboxamide (200 mg, 182.25 μmol) in DCM (2 mL) was added Et₃N (55.33 mg, 546.76 μmol) and TBSOTf (96.35 mg, 364.51 μmol) at 0° C., then the mixture was stirred at 30° C. for 2 h. The reaction mixture was quenched by H₂O (30 mL) at 0° C., and then extracted with EtOAc (3×15 mL). The combined organic layers were washed with brine (20 mL), dried over Na₂SO₄, filtered and concentrated under reduced pressure to give a residue. The residue was purified by prep-TLC (10% MeOH/EtOAc) to give the product (150 mg, 57% yield) as a solid. LCMS (ESI) m/z [M+H] calcd for C₆₇H₉₁N₈O₉SSi: 1211.6; found: 1211.7.

### Step 2: Synthesis of (2R,3R)—N-(2-(((1 S)-2-(((6³S,4S)-2⁵-((tert-butyldimethylsilyl)oxy)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-1-cyclopentyl-2-oxoethyl)(methyl)amino)-2-oxoethyl)-3-cyclopropyl-N-methylaziridine-2-carboxamide

To a solution of (2R,3R)—N-(2-(((1 S)-2-(((6³S,4S)-2⁵-((tert-butyldimethylsilyl)oxy)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-1-cyclopentyl-2-oxoethyl)(methyl)amino)-2-oxoethyl)-3-cyclopropyl-N-methyl-1-((R)-p-tolylsulfinyl)aziridine-2-carboxamide (300 mg, 247.60 μmol) in THE (3 mL) at −70° C. was added MeMgBr (3 M, 660.27 μL). The mixture was stirred at −70° C. for 1 h. The reaction was quenched by sat. aq. NH₄Cl (30 mL) at 0° C., and the mixture was extracted with EtOAc (3×15 mL). The combined organic layers were washed with brine (20 mL), dried over anhydrous Na₂SO₄, filtered and concentrated under reduced pressure. The residue was purified by reverse phase chromatography (80→98% MeCN/H₂O 0.1% NH₄HCO₃) and lyophilized to give the product (56.05 mg, 76% yield) as a solid. LCMS (ESI) m/z [M+H] calcd for C₆₀H₈₅N₈O₈Si: 1073.63; found: 1073.6

### Synthesis of (2R,3R)—N-(2-(((1S)-1-cyclopentyl-2-(((6³S,4S)-1¹-ethyl-1²-(2-((S)-1-methoxyethyl)pyridin-3-yl)-10,10-dimethyl-5,7-dioxo-2⁵-((triethylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methylaziridine-2-carboxamide

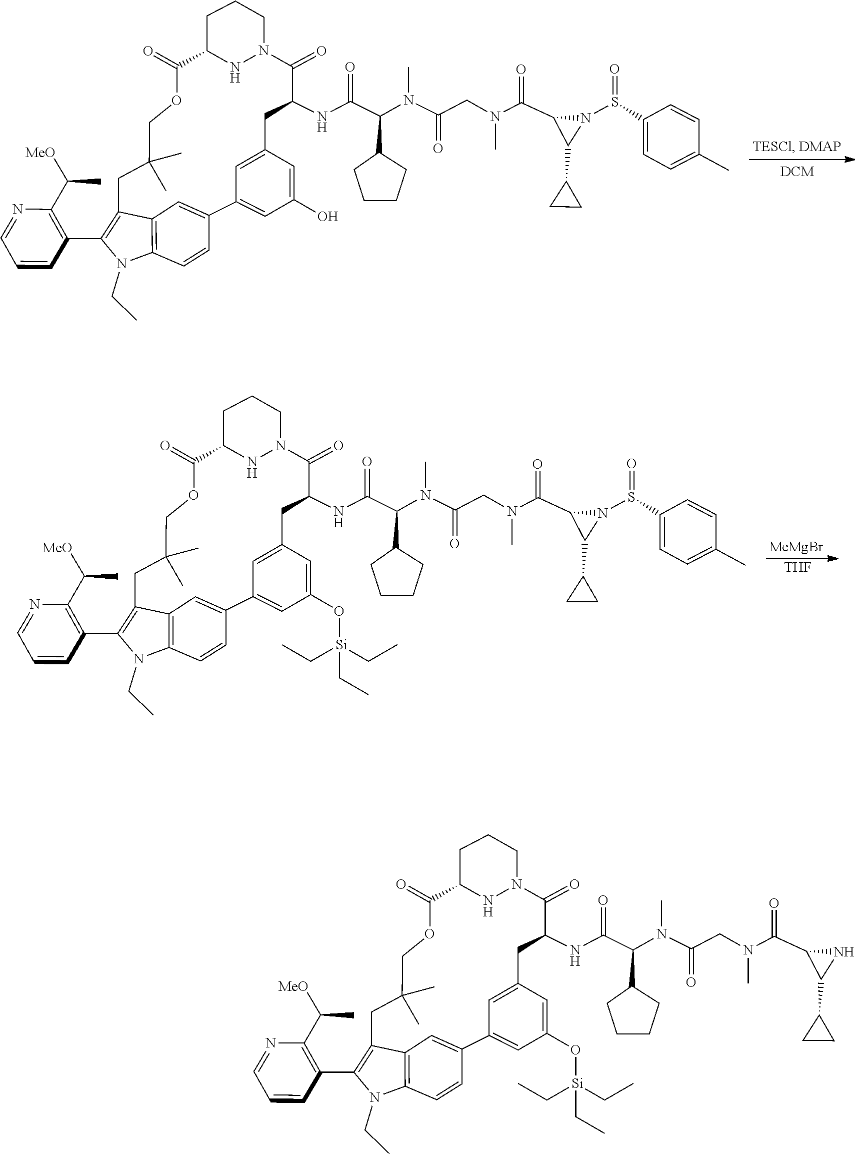

### Step 1: Synthesis of (2R,3R)—N-(2-(((1 S)-1-cyclopentyl-2-(((6³S,4S)-1¹-ethyl-1²-(2-((S)-1-methoxyethyl)pyridin-3-yl)-10,10-dimethyl-5,7-dioxo-2⁵-((triethylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsulfinyl)aziridine-2-carboxamide

To a solution of (2R,3R)—N-(2-(((1 S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsulfinyl)aziridine-2-carboxamide (500 mg, 455.64 μmol) and DMAP (244.11 mg, 2.28 mmol) in DCM (5 mL) was added chloro(triethyl)silane (206.02 mg, 1.37 mmol), then the mixture was stirred at room temperature for 12 h. The reaction mixture was quenched with H₂O (10 mL) at 0° C., and then extracted with EtOAc (3×10 mL). The combined organic layers were washed with brine (3×10 mL), dried over Na₂SO₄, filtered and concentrated under reduced pressure. The residue was purified by silica gel column chromatography (0→100% EtOAc/pet. ether) to give the product (280 mg, 36% yield) as a solid. LCMS (ESI) m/z [M+H] calcd for C₆₇H₉₁N₈O₉SSi:1211.63; found: 1211.8.

### Step 2: Synthesis of (2R,3R)—N-(2-(((1 S)-1-cyclopentyl-2-(((6³S,4S)-1¹-ethyl-1²-(2-((S)-1-methoxyethyl)pyridin-3-yl)-10,10-dimethyl-5,7-dioxo-2⁵-((triethylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methylaziridine-2-carboxamide

To a solution of (2R,3R)—N-(2-(((1 S)-1-cyclopentyl-2-(((6³S,4S)-1¹-ethyl-1²-(2-((S)-1-methoxyethyl)pyridin-3-yl)-10,10-dimethyl-5,7-dioxo-2⁵-((triethylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsulfinyl)aziridine-2-carboxamide (260 mg, 214.59 μmol) in THE (2.5 mL) was added MeMgBr (3 M, 286.12 μL) at −70° C., then the mixture was stirred at −70° C. for 1 h. Sat. aq. NH₄Cl (10 mL) was added to the reaction mixture dropwise at −70° C. The cooling bath was removed, and the reaction mixture was warmed to room temperature. The mixture was extracted with EtOAc (3×20 mL) and the combined organic layer was concentrated under reduced pressure. The residue was purified by reverse phase chromatography (75→98% MeCN/H₂O 0.1% NH₄HCO₃) to give the product (40.27 mg, 17% yield) as a solid. LCMS (ESI) m/z [M+H] calcd for C₆₀H₈₅N₈O₈Si: 1073.63; found: 1073.5.

### Synthesis of (6³S,4S)-4-((S)-2-cyclopentyl-2-(2-((2R,3R)-3-cyclopropyl-N-methylaziridine-2-carboxamido)-N-methylacetamido)acetamido)-1-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl pivalate

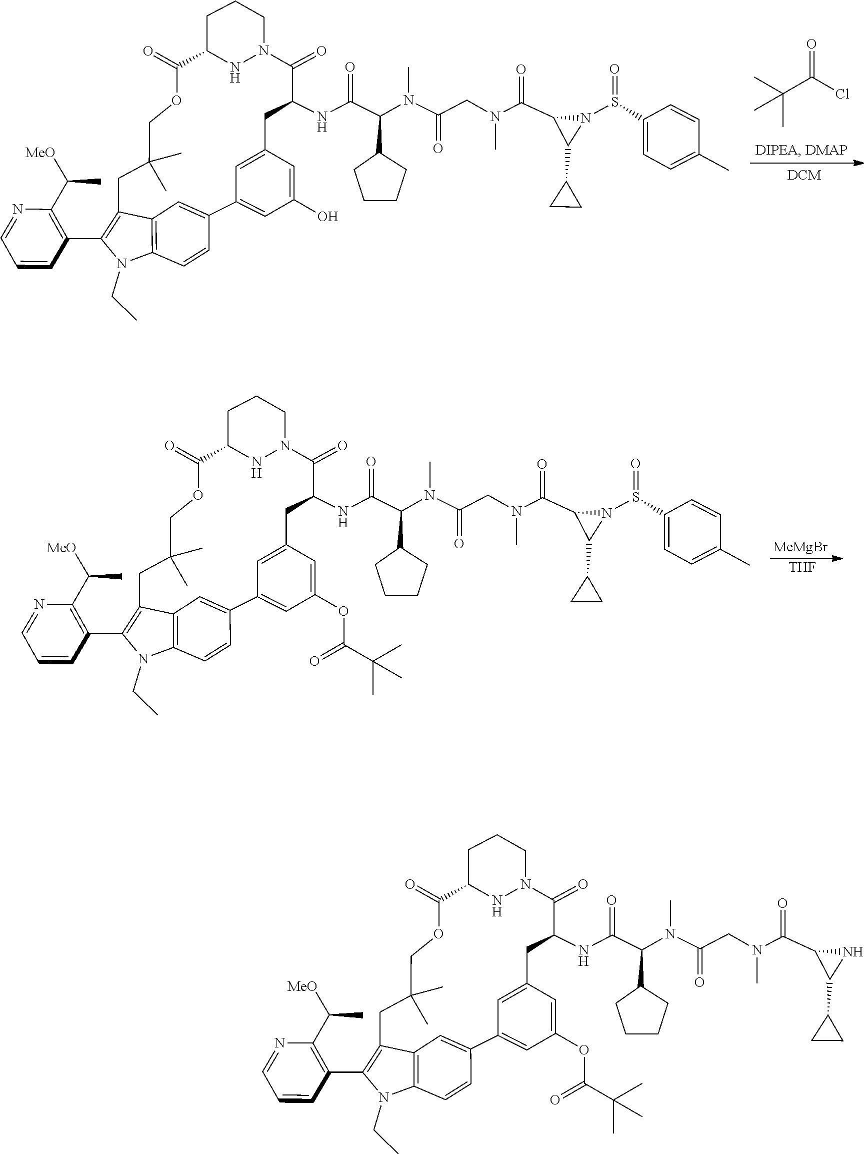

### Step 1: Synthesis of (6³S,4S)-4-((S)-2-cyclopentyl-2-(2-((2R,3R)-3-cyclopropyl-N-methyl-1-((R)-p-tolylsulfinyl)aziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-2⁵-yl pivalate

To a solution of (2R,3R)—N-(2-(((1 S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsulfinyl)aziridine-2-carboxamide (500 mg, 455.64 μmol) and DIPEA (176.66 mg, 1.37 mmol) in DCM (7.5 mL) was added 2,2-dimethylpropanoyl chloride (71.42 mg, 592.33 μmol) and DMAP (5.57 mg, 45.56 μmol) at 0° C. The mixture was stirred at room temperature for 1 h. The reaction mixture was quenched with H₂O (10 mL) at 0° C., and then extracted with EtOAc (3×10 mL). The combined organic layers were washed with brine (3×15 mL), dried over Na₂SO₄, filtered and concentrated under reduced pressure. The residue was purified by silica gel column chromatography (0→100% EtOAc/pet. ether) to give the product (450 mg, 76% yield) as a solid. LCMS (ESI) m/z [M+H] calcd for C₆₆H₈₅N₈O₁₀S: 1181.60; found: 1181.6.

### Step 2: (6³S,4S)-4-((S)-2-cyclopentyl-2-(2-((2R,3R)-3-cyclopropyl-N-methylaziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl pivalate

To a solution of (6³S,4S)-4-((S)-2-cyclopentyl-2-(2-((2R,3R)-3-cyclopropyl-N-methyl-1-((R)-p-tolylsulfinyl)aziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl pivalate (330 mg, 279.31 μmol) in THE (3 mL) was added MeMgBr (3 M, 744.83 μL) at −70° C., then the mixture was stirred at −70° C. for 1 h. Sat. aq. NH₄Cl (10 mL) was added to the reaction mixture dropwise at −70° C. The cooling bath was removed, and the reaction mixture was warmed to room temperature. The mixture was extracted with EtOAc (3×20 mL) and the combined organic layers were concentrated under reduced pressure. The residue was purified by reverse phase chromatography (55→85% MeCN/H₂O 0.1% NH₄HCO₃) and lyophilized to give the product (120 mg, 41% yield) as a solid. LCMS (ESI) m/z [M+H] calcd for C₅₉H₇₉N₈O₉: 1043.60; found: 1043.6.

### Synthesis of (6³S,4S)-4-((S)-2-cyclopentyl-2-(2-((2R,3R)-3-cyclopropyl-N-methylaziridine-2-carboxamido)-N-methylacetamido)acetamido)-1-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl methyl carbonate

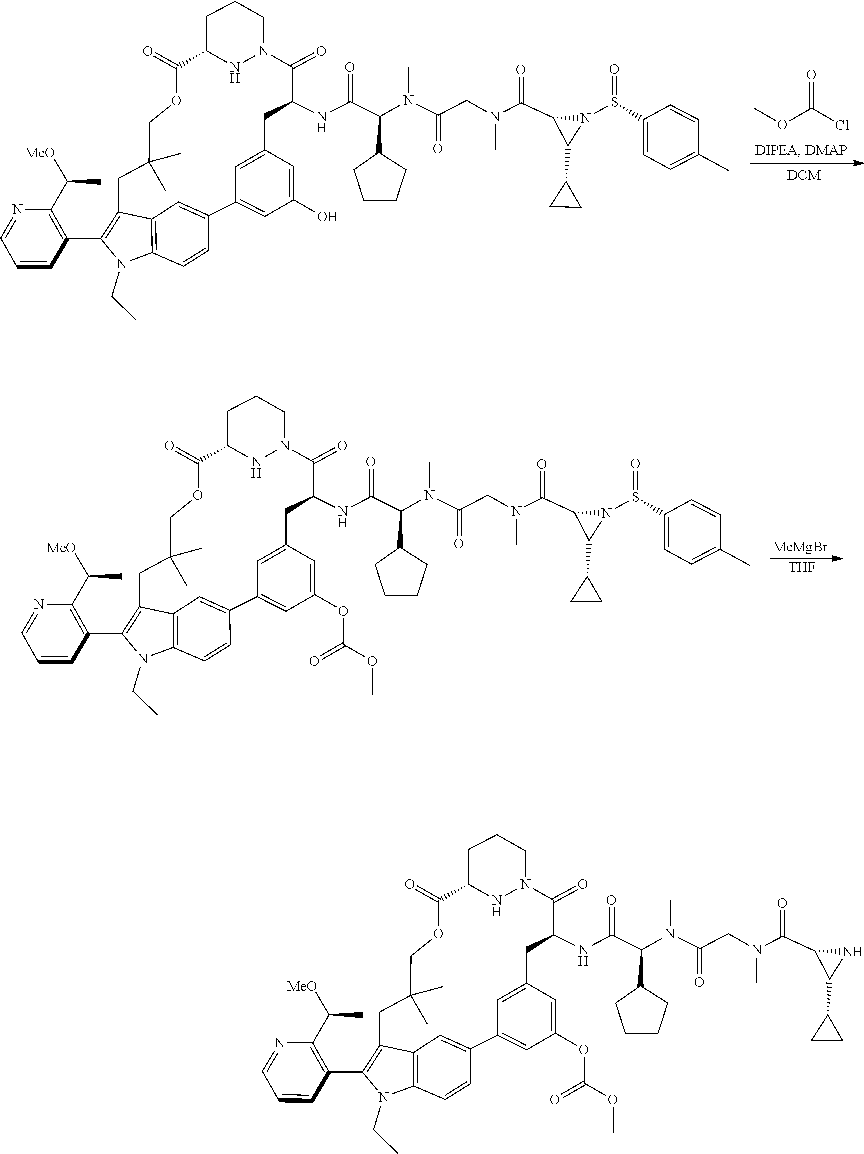

### Step 1: Synthesis of (6³S,4S)-4-((S)-2-cyclopentyl-2-(2-((2R,3R)-3-cyclopropyl-N-methyl-1-((R)-p-tolylsμLfinyl)aziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl methyl carbonate

To a solution of (2R,3R)—N-(2-(((1 S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsμLfinyl)aziridine-2-carboxamide (500 mg, 455.64 μmol) and DIPEA (176.66 mg, 1.37 mmol) in DCM (7.5 mL) was added methyl carbonochloridate (55.97 mg, 592.33 μmol) and DMAP (5.57 mg, 45.56 μmol) at 0° C. The mixture was stirred at room temperature for 1 h. The reaction mixture was quenched by H₂O (10 mL) at 0° C., and then extracted with EtOAc (3×10 mL). The combined organic layers were washed with brine (3×15 mL), dried over Na₂SO₄, filtered and concentrated under reduced pressure. The residue was purified by silica gel column chromatography (0→100% EtOAc/pet. ether) to give the product (450 mg, 82% yield) as a solid. LCMS (ESI) m/z [M+H] calcd for C₆₃H₇₉N₈O₁₁S: 1155.55; found: 1155.6.

### Step 2: Synthesis of (6³S,4S)-4-((S)-2-cyclopentyl-2-(2-((2R,3R)-3-cyclopropyl-N-methylaziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl methyl carbonate

To a solution of (6³S,4S)-4-((S)-2-cyclopentyl-2-(2-((2R,3R)-3-cyclopropyl-N-methyl-1-((R)-p-tolylsμLfinyl)aziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl methyl carbonate (170 mg, 147.13 μmol) in THE (1.7 mL) was added MeMgBr (3 M, 392.36 μL) at −70° C., then the mixture was stirred at −70° C. for 1 h. Sat. aq. NH₄Cl (10 mL) was added to the reaction mixture dropwise at −70° C. The cooling bath was removed, and the reaction mixture was warmed to room temperature. The mixture was extracted with EtOAc (3×20 mL) and the combined organic layers were concentrated under reduced pressure. The residue was purified by reverse phase chromatography (35→65% MeCN/H₂O 0.1% NH₄HCO₃) to give the product (50.12 mg, 33% yield) as a solid. LCMS (ESI) m/z [M+H] calcd for C₅₆H₇₃N₈O₁₀: 1017.55; found: 1017.5.

### Synthesis of (6³S,4S)-4-((S)-2-cyclopentyl-2-(2-((2R,3R)-3-cyclopropyl-N-methylaziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl isobutyrate

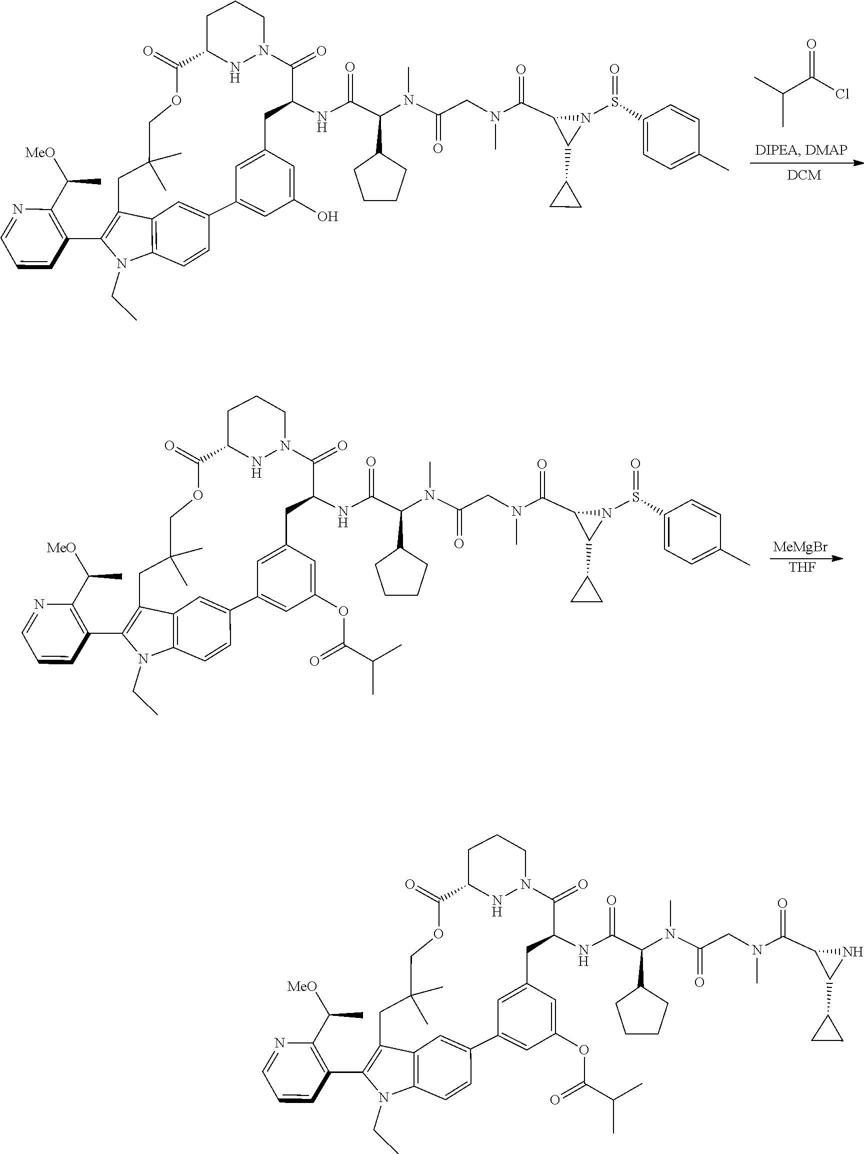

### Step 1: Synthesis of (6³S,4S)-4-((S)-2-cyclopentyl-2-(2-((2R,3R)-3-cyclopropyl-N-methyl-1-((R)-p-tolylsμLfinyl)aziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl isobutyrate

To a solution of (2R,3R)—N-(2-(((1 S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsμLfinyl)aziridine-2-carboxamide (7.8 g, 7.11 mmol) and DIPEA (2.76 g, 21.32 mmol) in DCM (120 mL) was added 2-methylpropanoyl chloride (984.56 mg, 9.24 mmol) and DMAP (86.84 mg, 710.79 μmol) at 0° C. The mixture was stirred at room temperature for 1 h. The reaction mixture was poured into cold H₂O (360 mL) and extracted with DCM (3×120 mL). The combined organic layers were washed with brine (100 mL), dried over Na₂SO₄, filtered and concentrated under reduced pressure to give a residue. The crude product was purified by silica gel column chromatography (0→100% EtOAc/pet. ether) to give the product (6.7 g, 81% yield) as a solid. LCMS (ESI) m/z [M/2+H] calcd for C₆₅H₈₃N₈O₁₀S: 584.7; found: 584.7.

### Step 2: Synthesis of (6³S,4S)-4-((S)-2-cyclopentyl-2-(2-((2R,3R)-3-cyclopropyl-N-methylaziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl isobutyrate

To a solution of (6³S,4S)-4-((S)-2-cyclopentyl-2-(2-((2R,3R)-3-cyclopropyl-N-methyl-1-((R)-p-tolylsμLfinyl)aziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl isobutyrate (6.2 g, 5.31 mmol) in THE (75 mL) was added MeMgBr (3 M, 7.97 mL) at −70° C. The mixture was stirred at −70° C. for 30 min. The reaction mixture was quenched by addition of sat. aq. NH₄Cl (80 mL) at −70° C., and then diluted with H₂O (80 mL) and extracted with EtOAc (3×50 mL). The combined organic layers were washed with brine (30 mL), dried over Na₂SO₄, filtered and concentrated under reduced pressure to give the crude product. The crude product was purified by reverse phase chromatography (35→70% MeCN/H₂O 0.1% NH₄HCO₃). The eluent was lyophilized to give the product (4.77 g, 83% yield) as a solid. LCMS (ESI) m/z [M+H] calcd for C₅₈H₇₇N₈O₉: 1029.58; found: 1029.6

### Synthesis of (6³S,4S)-4-((S)-2-cyclopentyl-2-(2-((2R,3R)-3-cyclopropyl-N-methylaziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl acetate

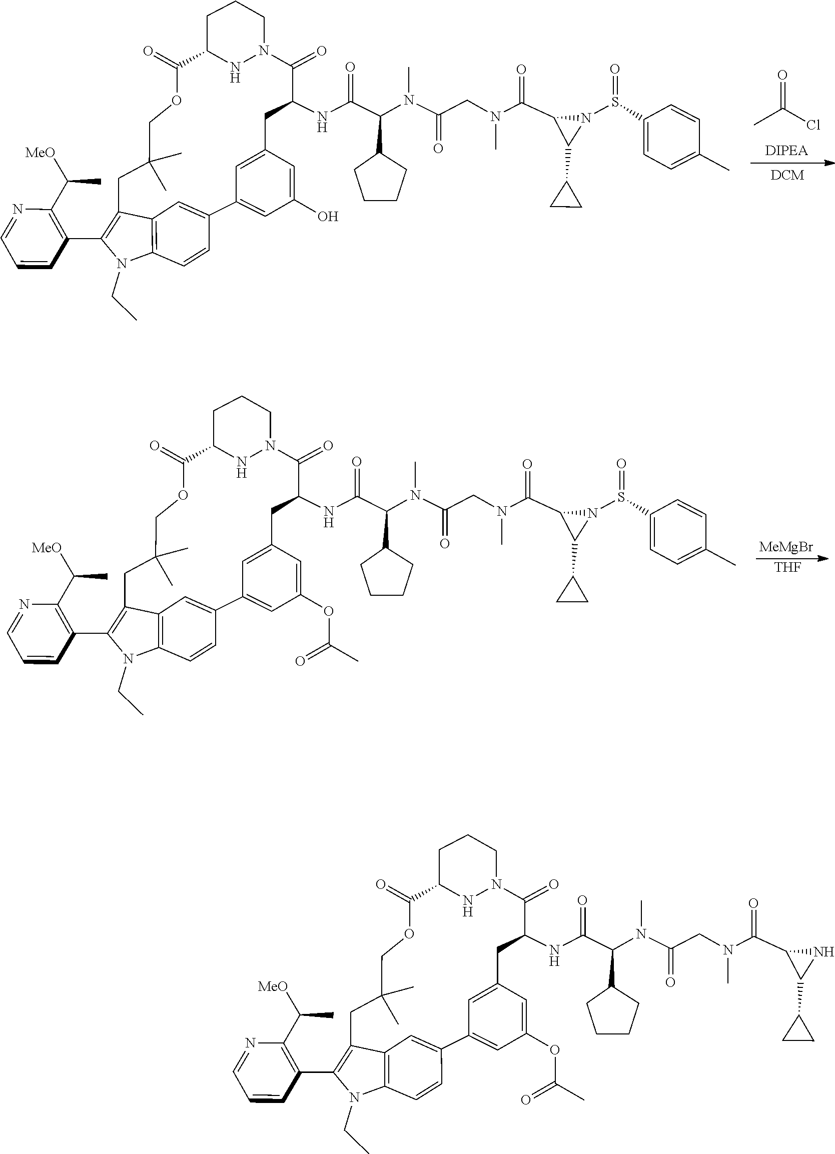

### Step 1: Synthesis of (6³S,4S)-4-((S)-2-cyclopentyl-2-(2-((2R,3R)-3-cyclopropyl-N-methyl-1-((R)-p-tolylsulfinyl)aziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl acetate

To a solution of (2R,3R)—N-(2-(((1 S)-1-cyclopentyl-2-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)(methyl)amino)-2-oxoethyl)-3-cyclopropyl-N-methyl-1-((R)-p-tolylsulfinyl)aziridine-2-carboxamide (110 mg, 100.24 μmol) and DIPEA (38.87 mg, 300.72 μmol) in DCM (1.1 mL) was added acetyl chloride (9.44 mg, 120.29 μmol) at 0° C. The mixture was stirred at room temperature for 1 h. The reaction mixture was poured into cold H₂O (20 mL) and extracted with DCM (3×10 mL). The combined organic layers were washed with brine (10 mL), dried over Na₂SO₄, filtered and concentrated under reduced pressure to give a residue. The crude product was purified by silica gel column chromatography (0→1 00% EtOAc/pet. ether) to give the product (105 mg, 92% yield) as a solid. LCMS (ESI) m/z [M+H] calcd for C₆₄H₇₉N₈O₁₀S: 1139.6; found: 1139.6.

### Step 2: Synthesis of (6³S,4S)-4-((S)-2-cyclopentyl-2-(2-((2R,3R)-3-cyclopropyl-N-methylaziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl acetate

To a solution of (6³S,4S)-4-((S)-2-cyclopentyl-2-(2-((2R,3R)-3-cyclopropyl-N-methyl-1-((R)-p-tolylsulfinyl)aziridine-2-carboxamido)-N-methylacetamido)acetamido)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-2⁵-yl acetate (100 mg, 87.77 μmol) in THE (1 mL) was added MeMgBr (3 M, 146.28 μL) at −70° C. The mixture was stirred at −70° C. for 1 h. The reaction mixture was quenched by the addition sat. aq. NH₄Cl (2 mL) at −70° C., and then diluted with H₂O (20 mL) and extracted with EtOAc (3×10 mL). The combined organic layers were washed with brine (10 mL), dried over Na₂SO₄, filtered and concentrated under reduced pressure to give crude product. The crude product was purified by reverse phase chromatography (45→75% MeCN/H₂O 0.1% NH₄HCO₃)) to give the product (20.8 mg, 24% yield) as a solid. LCMS (ESI) m/z [M+H] calcd for C₅₆H₇₃N₈O₉: 1001.55; found: 1001.5.

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

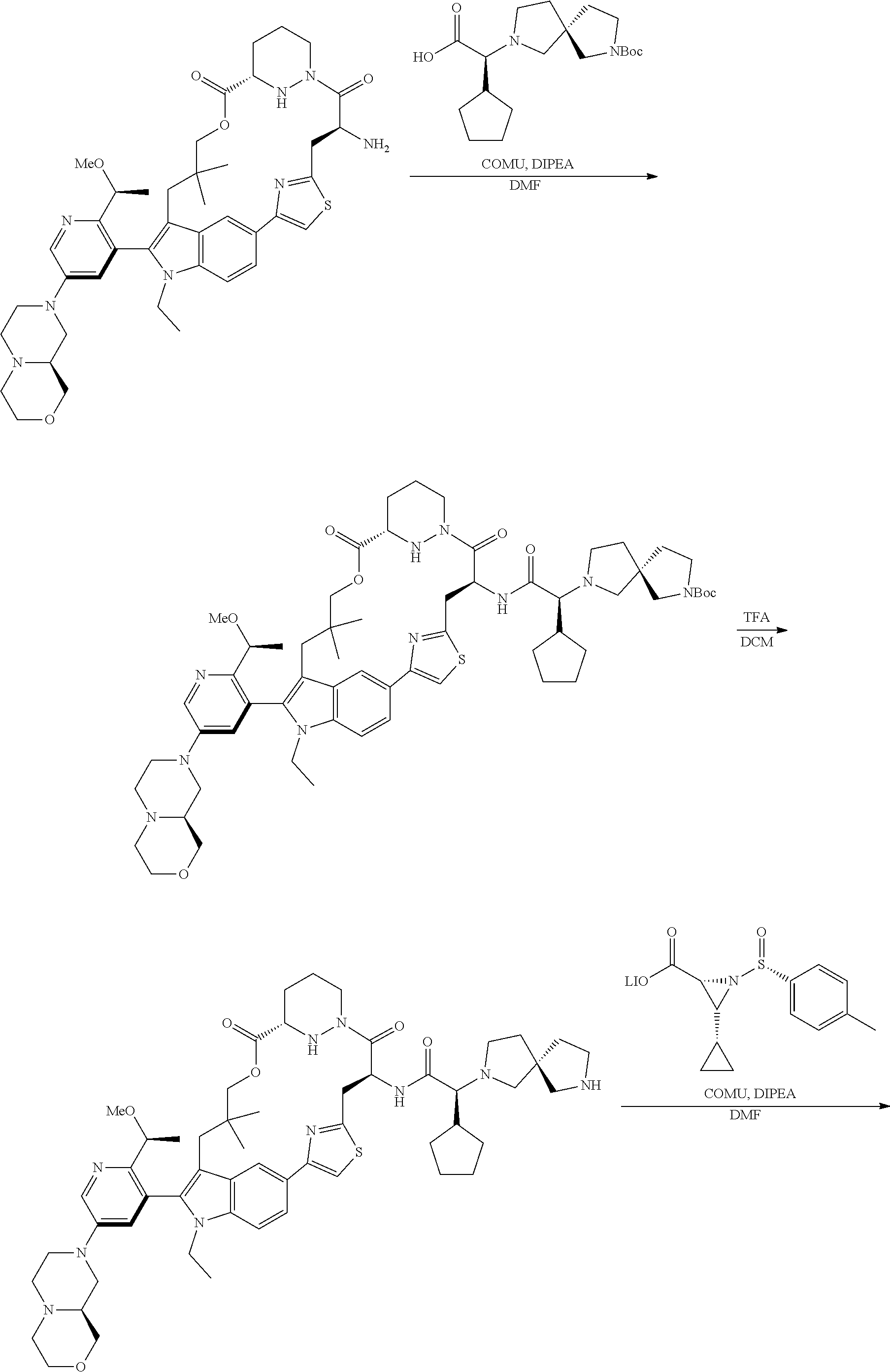

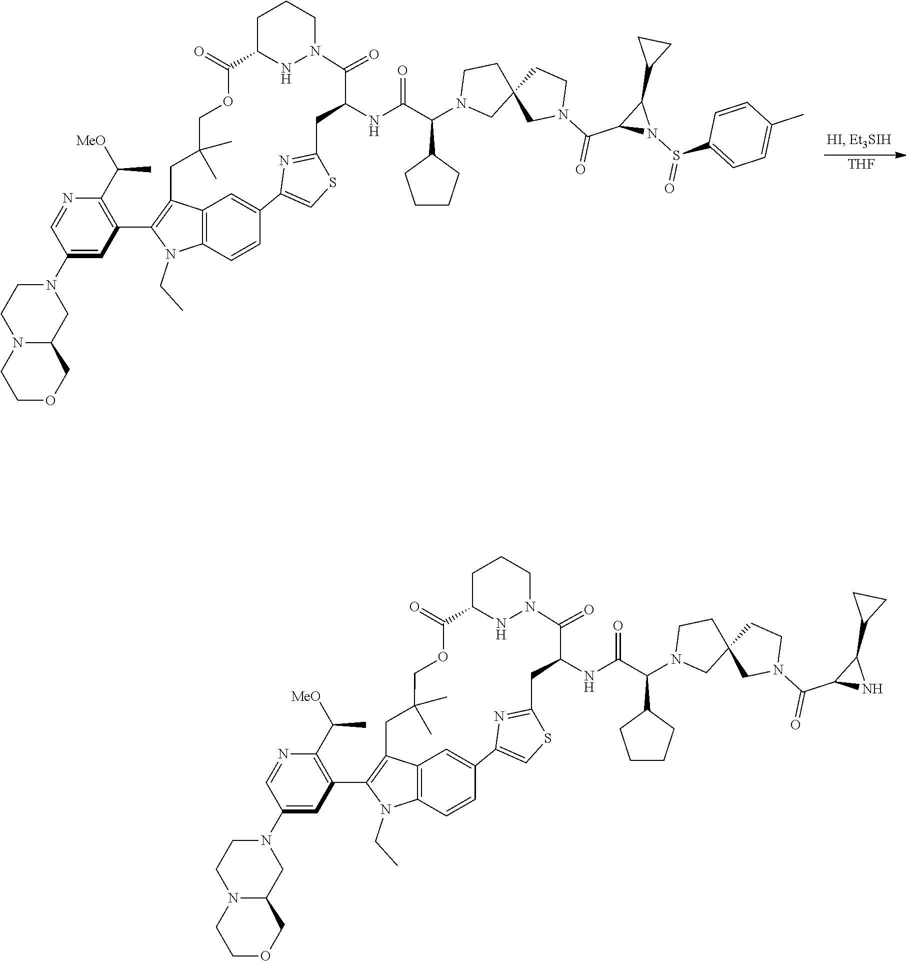

### Step 1: Synthesis of tert-butyl (5S)-7-((1 S)-1-cyclopentyl-2-(((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.4]nonane-2-carboxylate

To a stirred solution of (6³S,4S,Z)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (715 mg, 0.936 mmol), DIPEA (4840.19 mg, 37.440 mmol) and (S)-[(5S)-7-(tert-butoxycarbonyl)-2,7-diazaspiro[4.4]nonan-2-yl](cyclopentyl)acetic acid (429.01 mg, 1.217 mmol) in DMF (8 mL) was added COMU (441.07 mg, 1.030 mmol) in DMF (0.5 mL) dropwise at 0° C. under an argon atmosphere. The resulting mixture was stirred for 2 h at room temperature. The reaction was quenched by the addition of H₂O at 0° C. The resulting mixture was extracted with EtOAc (3×50 mL). The combined organic layers were washed with brine, dried over anhydrous Na₂SO₄. After filtration, the filtrate was concentrated under reduced pressure. The residue was purified by prep-TLC (10% MeOH/DCM) to afford the product (632 mg, 60% yield) as a solid. LCMS (ESI) m/z [M+H] calcd for C₆₀H₈₄N₁₀O₈S: 1105.63; found: 1105.8

### Step 2: Synthesis of (2S)-2-cyclopentyl-N-((63S,4S,2)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-2,7-diazaspiro[4.4]nonan-2-yl)acetamide

To a stirred solution of tert-butyl (5S)-7-((1 S)-1-cyclopentyl-2-(((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.4]nonane-2-carboxylate (659 mg) in DCM (6 mL) was added TFA (2 mL) dropwise at 0° C. under an argon atmosphere. The resulting mixture was stirred for 2 h at room temperature. The mixture was basified to pH 8 with sat. aq. NaHCO₃. The resulting mixture was extracted with EtOAc (3×50 mL). The combined organic layers were washed with brine, dried over anhydrous Na₂SO₄. After filtration, the filtrate was concentrated under reduced pressure. The crude product mixture was used in the next step directly without further purification.

LCMS (ESI) m/z [M+H] calcd for C₅₅H₇₆N₁₀O₆S: 1005.57; found: 1005.8

### Step 3: Synthesis of (2S)-2-((S)-7-((2R,3R)-1-((R)-tert-butylsulfinyl)-3-cyclopropylaziridine-2-carbonyl)-2,7-diazaspiro[4.4]nonan-2-yl)-2-cyclopentyl-N-((6³S,4S,2)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

To a stirred solution of (2S)-2-cyclopentyl-N-((63S,4S,2)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-2,7-diazaspiro[4.4]nonan-2-yl)acetamide (445 mg, 0.429 mmol), DIPEA (2215.52 mg, 17.160 mmol) and lithio (2R,3R)-3-cyclopropyl-1-[(R)-2-methylpropane-2-sulfinyl]aziridine-2-carboxylate (152.51 mg, 0.643 mmol) in DMF (4 mL) was added COMU (220.24 mg, 0.515 mmol) in DMF(1 mL) dropwise at 0° C. under an argon atmosphere. The resulting mixture was stirred for 2 h at room temperature. The reaction was quenched with H₂O at 0° C. The resulting mixture was extracted with EtOAc (3×50 mL). The combined organic layers were washed with brine, dried over anhydrous Na₂SO₄. After filtration, the filtrate was concentrated under reduced pressure. The residue was purified by prep-TLC (8% MeOH/DCM) to afford the product (410 mg, 69% yield) as a solid. LCMS (ESI) m/z [M+H] calcd for C₆₅H₉₁N₁₁O₈S₂: 1218.66; found: 1218.9

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1 (5,3)-indola-6(1,3)-pyridazinacycloundecaphane-4-yl)acetamide

To a stirred solution of (2S)-2-((S)-7-((2R,3R)-1-((R)-tert-butylsulfinyl)-3-cyclopropylaziridine-2-carbonyl)-2,7-diazaspiro[4.4]nonan-2-yl)-2-cyclopentyl-N-((6³S,4S,2)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide (600 mg, 0.492 mmol) in THE (6 mL) were added Et₃SiH (572.50 mg, 4.920 mmol) and HI (629.78 mg, 4.920 mmol) dropwise at 0° C. under an argon atmosphere. The resulting mixture was stirred for 30 min at 0° C. The reaction was quenched with DIPEA (600 mg) in EtOAc (100 mL) at 0° C. The resulting mixture was extracted with EtOAc (3×100 mL). The combined organic layers were washed with brine (3×30 mL), dried over anhydrous Na₂SO₄. After filtration, the filtrate was concentrated under reduced pressure. The residue was purified by prep-TLC (8% MeOH/DCM) to afford the product (200 mg, 36% yield) as a solid. LCMS (ESI) m/z [M+H] calcd C₆₁H₈₃N₁₁O₇S: 1114.63; found: 1113.0

### Synthesis of (2S)-2-cyclopentyl-N-((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-7-((R)-oxirane-2-carbonyl)-2,7-diazaspiro[4.4]nonan-2-yl)acetamide

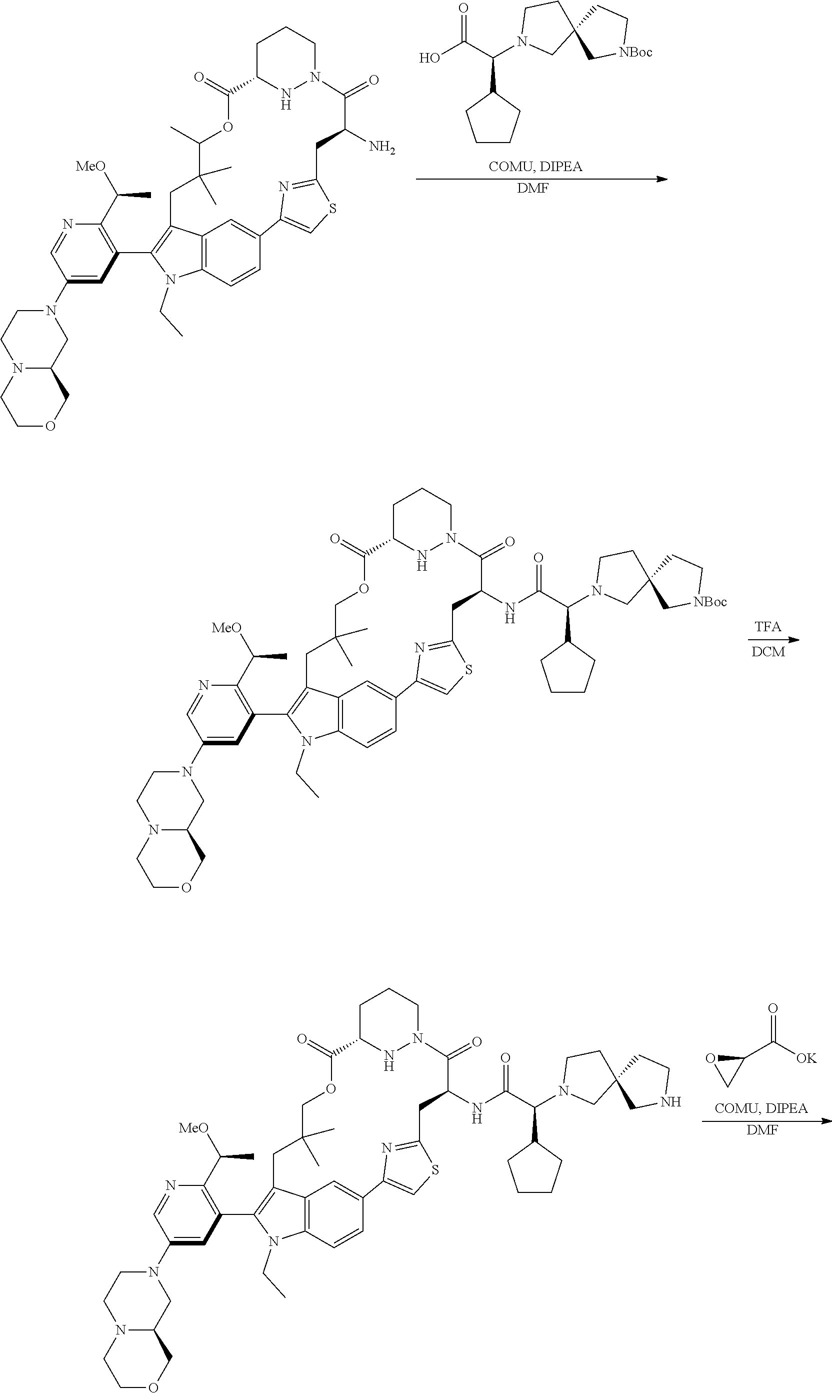

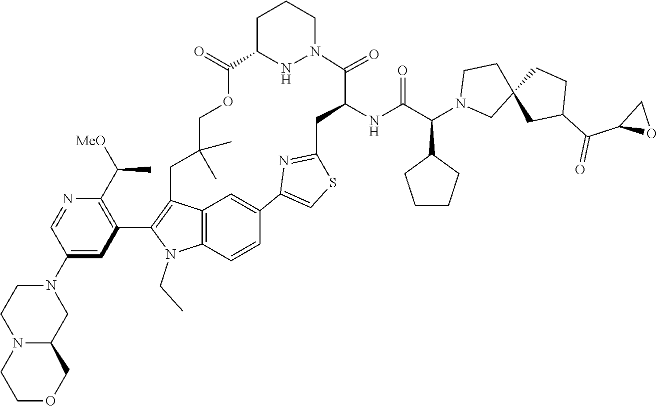

### Step 1: Synthesis of tert-butyl (5S)-7-((1 S)-1-cyclopentyl-2-(((6³S,4S,2)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.4]nonane-2-carboxylate

To a stirred mixture of (6³S,4S,2)-4-amino-1′-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (229 mg, 0.297 mmol) and DIPEA (1.54 g, 11.880 mmol) in DMF (3 mL) was added (S)-2-((S)-7-(tert-butoxycarbonyl)-2,7-diazaspiro[4.4]nonan-2-yl)-2-cyclopentylacetic acid (314.08 mg, 0.891 mmol) and COMU (254.41 mg, 0.594 mmol) dropwise at 0° C. under an argon atmosphere. The resulting mixture was stirred for 4 h at room temperature. The resulting mixture was extracted with EtOAc (1×50 mL). The combined organic layers were washed with brine (3×50 mL), dried over anhydrous Na₂SO₄. After filtration, the filtrate was concentrated under reduced pressure. The resulting mixture was concentrated under reduced pressure. The residue was purified by prep-TLC (10% MeOH/DCM) to afford the product (158 mg, 48% yield) as a solid. LCMS (ESI) m/z [M+H] calcd for C₆₀H₈₄N₁₀O₈S: 1105.6; found: 1106.0

### Step 2: Synthesis of (2S)-2-cyclopentyl-N-((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-2,7-diazaspiro[4.4]nonan-2-yl)acetamide

A mixture tert-butyl (5S)-7-((1S)-1-cyclopentyl-2-(((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.4]nonane-2-carboxylate (158 mg, 0.143 mmol) and TFA (0.8 mL, 8.161 mmol) in DCM (1.6 mL) was stirred for 1 h at 0° C. under an argon atmosphere. The resulting mixture was concentrated under reduced pressure and diluted with EtOAc (100 mL). The mixture was neutralized to pH 7 with sat. aq. NaHCO₃, the combined organic layers were washed with brine (3×100 mL), dried over anhydrous Na₂SO₄. After filtration, the filtrate was concentrated under reduced pressure to afford the product (158 mg, crude) as a solid. LCMS (ESI) m/z [M+H] calcd C₅₅H₇₆N₁₀O₆S: 1005.57; found: 1005.8

### Step 3: Synthesis of (2S)-2-cyclopentyl-N-((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-7-((R)-oxirane-2-carbonyl)-2,7-diazaspiro[4.4]nonan-2-yl)acetamide

To a stirred solution of (2S)-2-cyclopentyl-N-((6³S,4S,Z)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-2,7-diazaspiro[4.4]nonan-2-yl)acetamide (146 mg, 0.145 mmol) and DIPEA (750.77 mg, 5.800 mmol) in DMF (2 mL) were added potassium (2R)-oxirane-2-carboxylate (54.96 mg, 0.435 mmol) and COMU (124.39 mg, 0.290 mmol) dropwise at 0° C. under an argon atmosphere. The final reaction mixture was stirred for 2 h at room temperature. The resulting mixture was diluted with EtOAc (50 mL). The resulting mixture was washed with brine (3×50 mL), dried over anhydrous Na₂SO₄. After filtration, the filtrate was concentrated under reduced pressure. The crude product was purified by reverse phase chromatography (38→54% MeCN/H₂O 0.1% NH₄HCO₃) to afford the product (31.7 mg, 20% yield) as a solid. LCMS (ESI) m/z [M+H] calcd C₅₈H₇₈N₁₀O₈S: 1075.58; found: 1076.1

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S,Z)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

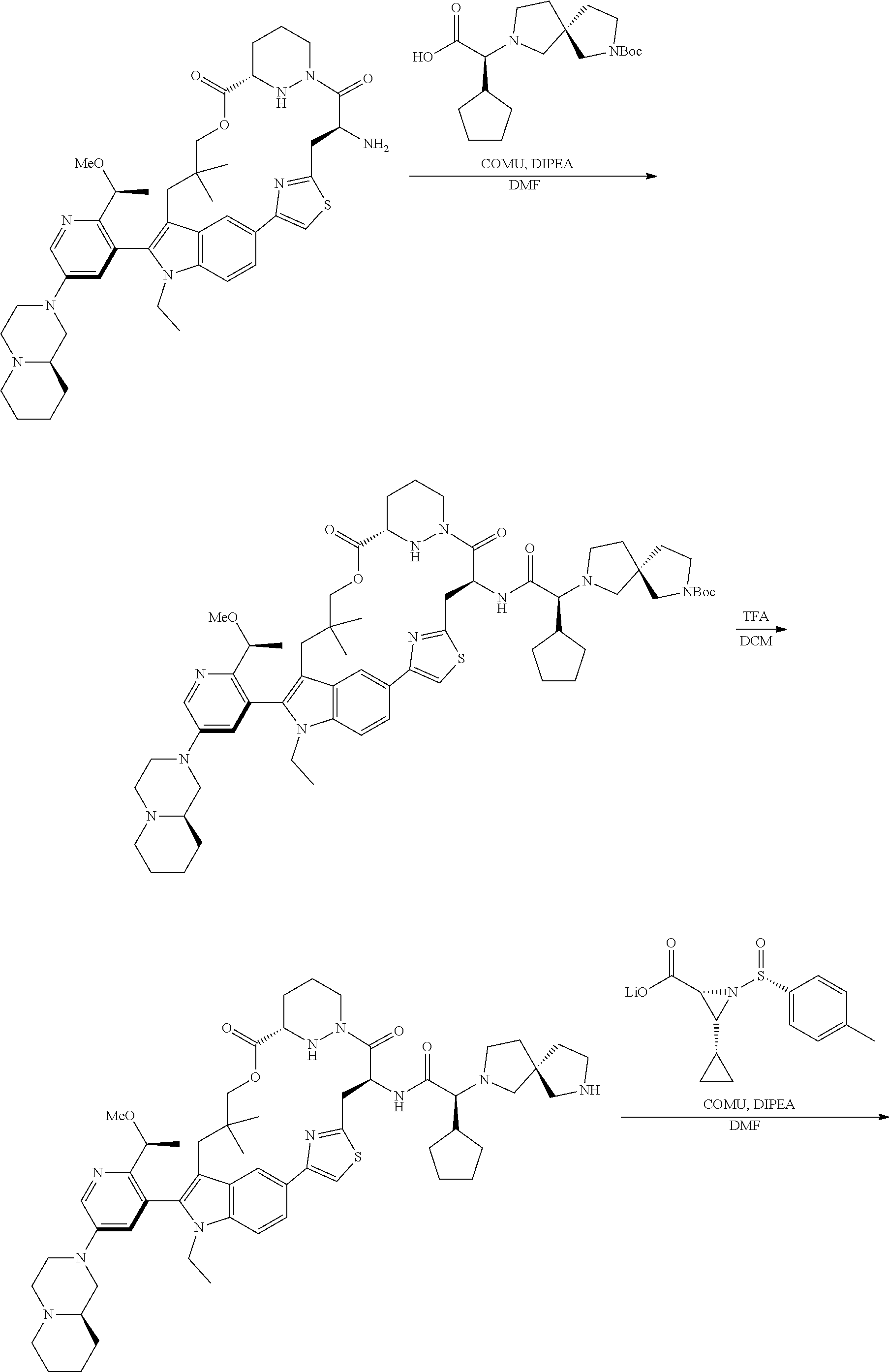

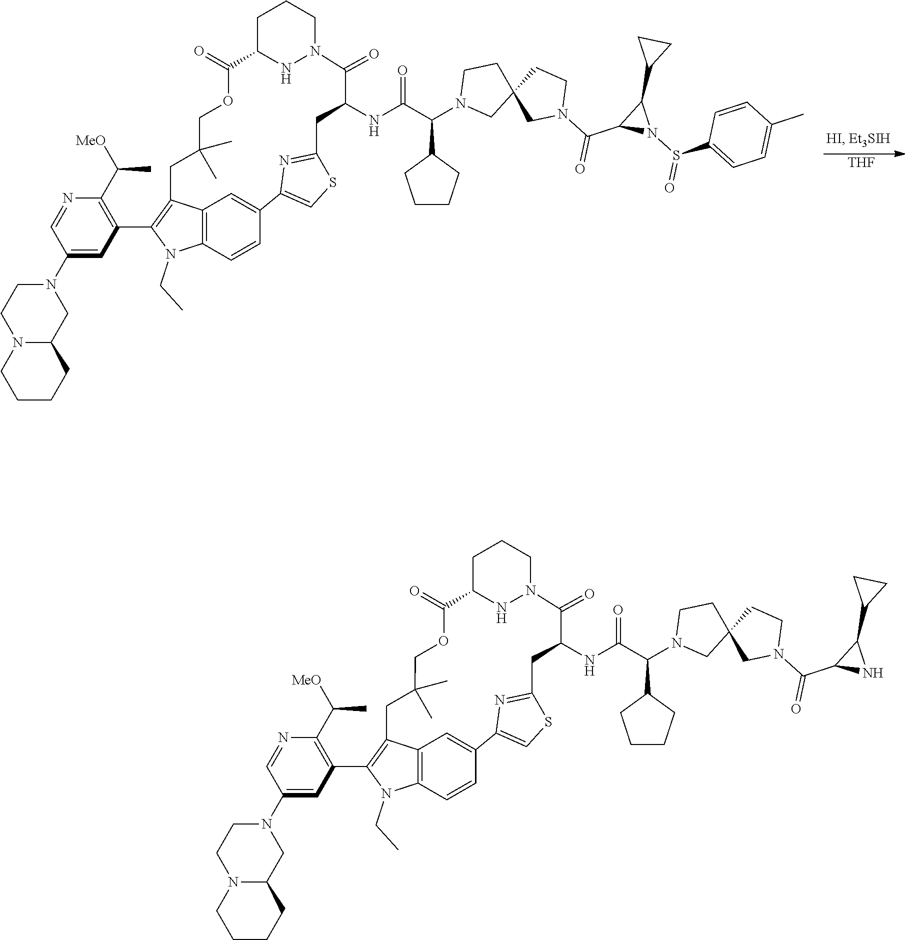

### Step 1: Synthesis of tert-butyl (5S)-7-((1S)-1-cyclopentyl-2-(((6³S,4S,Z)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.4]nonane-2-carboxylate

To a stirred solution of (6³S,4S,Z)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-2(4,2)-thiazola-1(5,3)-indola-6(1,3)-pyridazinacycloundecaphane-5,7-dione (720 mg, 0.936 mmol), DIPEA (4840.19 mg, 37.440 mmol) and (S)-[(5S)-7-(tert-butoxycarbonyl)-2,7-diazaspiro[4.4]nonan-2-yl](cyclopentyl)acetic acid (429.01 mg, 1.217 mmol) in DMF (8 mL) was added COMU (441.07 mg, 1.030 mmol) in DMF (0.5 mL) dropwise at 0° C. under an argon atmosphere. The resulting mixture was stirred for 2 h at room temperature. The reaction was quenched by the addition of H₂O at 0° C. The resulting mixture was extracted with EtOAc and the combined organic layers were washed with brine, dried over anhydrous Na₂SO₄. After filtration, the filtrate was concentrated under reduced pressure. The residue was purified by prep-TLC (10% MeOH/DCM) to afford the product (620 mg, 60% yield) as a solid. LCMS (ESI) m/z [M+H] calcd for C₆₁H₈₆N₁₀O₇S: 1103.65; found:1103.9

### Step 2: Synthesis of (2S)-2-cyclopentyl-N-((6³S,4S,2)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-2,7-diazaspiro[4.4]nonan-2-yl)acetamide

To a stirred solution of tert-butyl (5S)-7-((1 S)-1-cyclopentyl-2-(((6³S,4S,Z)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)amino)-2-oxoethyl)-2,7-diazaspiro[4.4]nonane-2-carboxylate (620 mg) in DCM (6 mL) was added TFA (2 mL) dropwise at 0° C. The resulting mixture was stirred for 2 h at room temperature. The mixture was basified to pH 8 with sat. aq. NaHCO₃ the resulting mixture was extracted with EtOAc (3×100 mL). The combined organic layers were washed with brine, dried over anhydrous Na₂SO₄. After filtration, the filtrate was concentrated under reduced pressure to afford the product (500 mg, crude) which was used in the next step directly without further purification. LCMS (ESI) m/z [M+H] calcd C₅₆H₇₈N₁₀O₅S: 1003.60; found: 1003.8

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S,2)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

To a stirred solution of (2S)-2-cyclopentyl-N-((6³S,4S,Z)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)-2-((S)-2,7-diazaspiro[4.4]nonan-2-yl)acetamide (430 mg, 0.429 mmol), DIPEA (2215.52 mg, 17.160 mmol) and lithio (2R,3R)-3-cyclopropyl-1-(R)-2-methylpropane-2-sulfinyl]aziridine-2-carboxylate (152.51 mg, 0.643 mmol) in DMF (4 mL) was added COMU (220.24 mg, 0.515 mmol) in DMF (1 mL) dropwise at 0° C. under an argon atmosphere. The resulting mixture was stirred for 2 h at room temperature. The reaction was quenched with H₂O at 0° C. The resulting mixture was extracted with EtOAc the combined organic layers were washed with brine, dried over anhydrous Na₂SO₄. After filtration, the filtrate was concentrated under reduced pressure. The residue was purified by prep-TLC (8% MeOH/DCM) to afford the product (400 mg, 69% yield) as a solid. LCMS (ESI) m/z [M+H] calcd C₆₆H₉₃N₁₁O₇S₂: 1216.68; found:1217.0

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S,Z)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide

To a stirred solution of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S,Z)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-thiazola-1(5,3)-indola-6(1,3)-pyridazinacycloundecaphane-4-yl)acetamide (480 mg, 0.395 mmol) in THE (5 mL) were added Et₃SiH (458.74 mg, 3.950 mmol) and HI (504.64 mg, 3.950 mmol) dropwise at 0° C. under an argon atmosphere. The resulting mixture was stirred for 30 min at 0° C. The reaction was quenched with DIPEA (500 mg) in EtOAc (100 mL) at 0° C. and extracted with EtOAc (200 mL). The combined organic layers were washed with brine (60 mL), dried over anhydrous Na₂SO₄. After filtration, the filtrate was concentrated under reduced pressure. The residue was purified by prep-TLC (10% MeOH/DCM) to afford the product (123 mg, 28% yield) as a solid. LCMS (ESI) m/z [M+H] calcd C₆₂H₈₅N₁₁O₆S: 1112.65; found: 1113.0

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-12-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

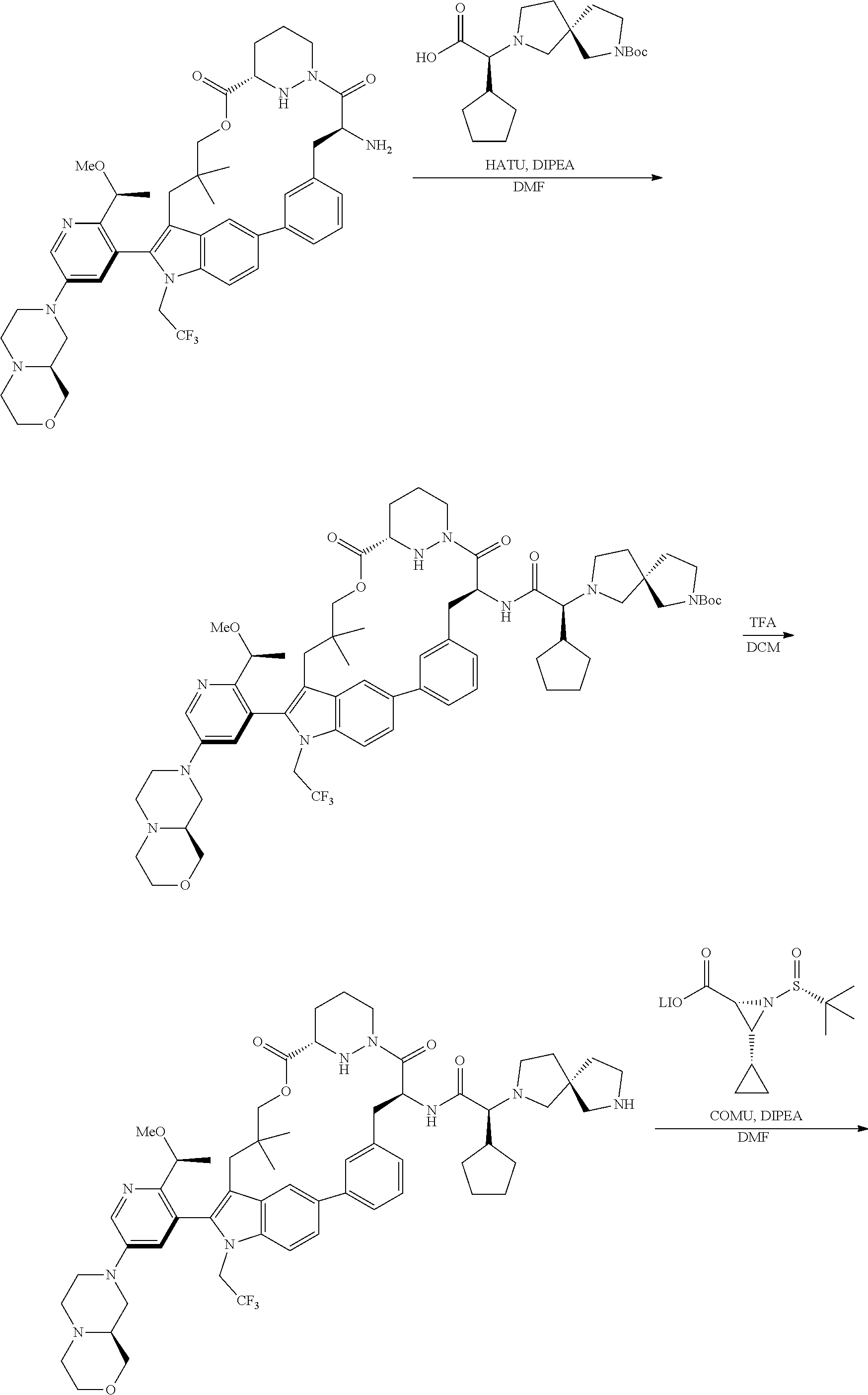

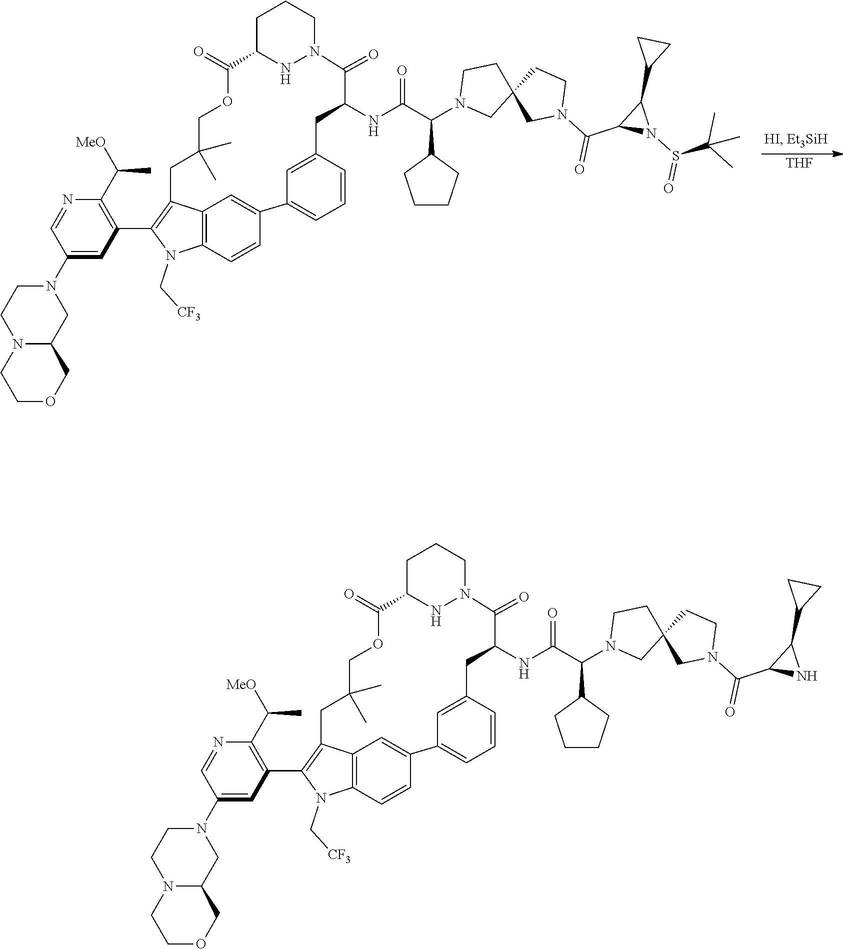

### Step 1: Synthesis of tert-butyl (5S)-7-((1 S)-1-cyclopentyl-2-(((6³S,4S)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-2-oxoethyl)-2,7-diazaspiro[4.4]nonane-2-carboxylate

To a stirred solution of (6³S,4S)-4-amino-1²-(5-((S)-hexahydropyrazino[2,1-c][1,4]oxazin-8(1H)-yl)-2-((S)-1-methoxyethyl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (380 mg, 0.465 mmol), DIPEA (2398 mg, 18.595 mmol) and (S)-2-((S)-7-(tert-butoxycarbonyl)-2,7-diazaspiro[4.4]nonan-2-yl)-2-cyclopentylacetic acid (253.81 mg, 0.721 mmol) in DMF (5 mL) was added HATU (229.64 mg, 0.605 mmol) dropwise at 0° C. under an argon atmosphere. The resulting mixture was stirred for 1.5 h at room temperature then diluted with H₂O. The resulting mixture was extracted with DCM. The combined organic layers were washed with brine, dried over anhydrous Na₂SO₄. After filtration, the filtrate was concentrated under reduced pressure. The residue was purified by silica gel column chromatography (10% MeOH/DCM) to afford the product (560 mg, crude) as a solid. LCMS (ESI) m/z [M+H] calcd C₆₃H₈₄F₃N₉O₈: 1152.65; found: 1152.4

### Step 2: Synthesis of (2S)-2-cyclopentyl-N-((6³S,4S)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

To a stirred solution of tert-butyl (5S)-7-((1 S)-1-cyclopentyl-2-(((6³S,4S)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-2-oxoethyl)-2,7-diazaspiro[4.4]nonane-2-carboxylate (680 mg, 0.590 mmol) in DCM (14 mL) was added TFA (3.5 mL) dropwise at 0° C. under an argon atmosphere. The resulting mixture was stirred for 1 h at 0° C. The residue was basified to pH 8 with sat. aq. NaHCO₃, the resulting mixture was extracted with EtOAc. The combined organic layers were washed with brine, dried over anhydrous Na₂SO₄. After filtration, the filtrate was concentrated under reduced pressure to afford the product (620 mg, crude) as a solid. LCMS (ESI) m/z [M+H] calcd C₅₈H₇₆F₃N₉O₆: 1052.59; found: 1052.8

### Step 3: Synthesis of tert-butyl((1 S,2R,3R)-2-((5S)-7-((1 S)-1-cyclopentyl-2-(((6³S,4S)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-2-oxoethyl)-2,7-diazaspiro[4.4]nonane-2-carbonyl)-3-cyclopropylaziridin-1-ium-1-yl)-l3-sulfanolate

To a stirred solution of (2S)-2-cyclopentyl-N-((6³S,4S)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (500 mg, 0.475 mmol), DIPEA (2456.40 mg, 19.00 mmol) and lithium (2R,3R)-1-((R)-tert-butylsulfinyl)-3-cyclopropylaziridine-2-carboxylate (146.54 mg, 0.617 mmol) in DMF (5 mL) was added COMU (244.19 mg, 0.570 mmol) dropwise at 0° C. under a nitrogen atmosphere. The resulting mixture was stirred for 2 h at room temperature. The resulting mixture was diluted with H₂O and extracted with EtOAc. The combined organic layers were washed with brine, dried over anhydrous Na₂SO₄. After filtration, the filtrate was concentrated under reduced pressure. The residue was purified by prep-TLC (6% MeOH/DCM) to afford the product (400 mg, 66% yield) as a solid. LCMS (ESI) m/z [M+H] calcd for C₆₈H₉₂F₃N₁₀O₈S: 1266.69; found: 1265.9

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-12-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a stirred solution of tert-butyl((1 S,2R,3R)-2-((5S)-7-((1 S)-1-cyclopentyl-2-(((6³S,4S)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-2-oxoethyl)-2,7-diazaspiro[4.4]nonane-2-carbonyl)-3-cyclopropylaziridin-1-ium-1-yl)-l3-sulfanolate (460 mg, 0.364 mmol) in THE (6 mL) was added Et₃SiH (422 mg, 3.640 mmol) dropwise at 0° C. under an argon atmosphere. To the above mixture was added HI (278 mg, 2.184 mmol) dropwise at 0° C. The resulting mixture was stirred for additional 30 min. The reaction was quenched by the addition of EtOAc (20 mL containing 470 mg of DIPEA) at 0° C. The resulting mixture was diluted with cold H₂O and extracted with EtOAc. The combined organic layers were washed with brine, dried over anhydrous MgSO₄. After filtration, the filtrate was concentrated under reduced pressure. The residue was purified by prep-TLC (8% DCM/MeOH with 0.1% Et₃N) to afford (149.5 mg, 35% yield) as a solid. LCMS (ESI) m/z [M+H] calcd for C₆₄H₈₃F₃N₁₀O₇: 1161.65; found: 1162.1

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-12-(2-((S)-1-methoxyethyl)-5-((R)-octahydro-2H-pyrido[1,2-a]pyrazin-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

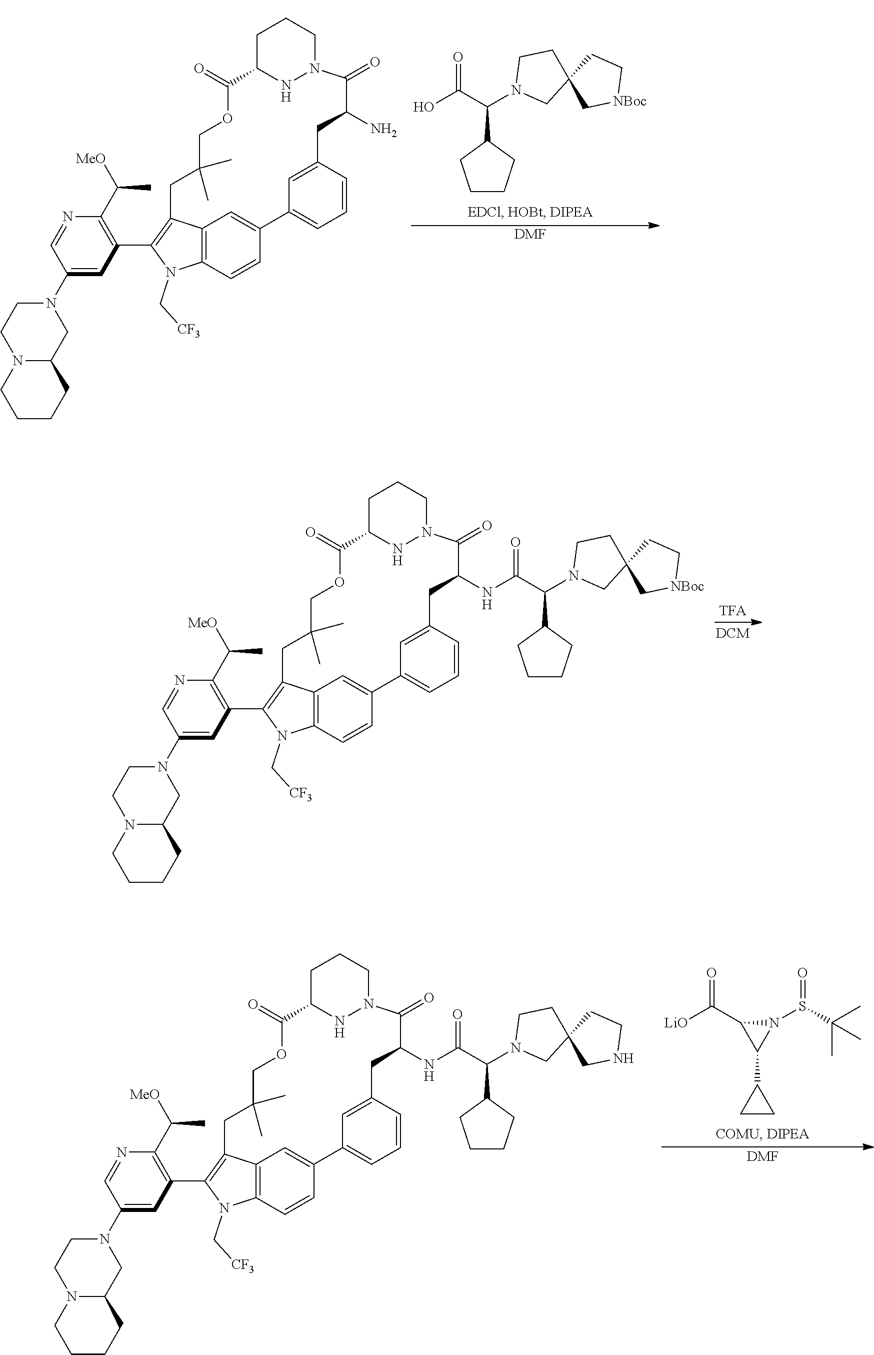

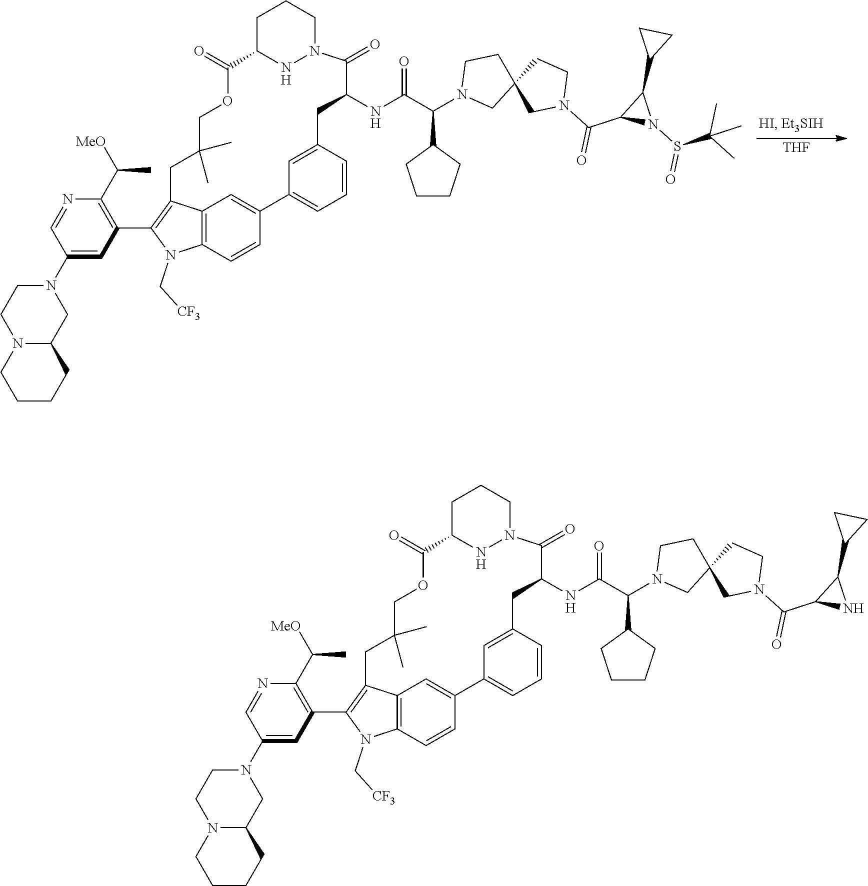

### Step 1: Synthesis of tert-butyl (5S)-7-((1S)-1-cyclopentyl-2-(((6³S,4S)-1²-(2-((S)-1-methoxyethyl)-5-((R)-octahydro-2H-pyrido[1,2-a]pyrazin-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

To a stirred mixture of (6³S,4S)-4-amino-12-(2-((S)-1-methoxyethyl)-5-((R)-octahydro-2H-pyrido[1,2-a]pyrazin-2-yl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (800 mg, 0.980 mmol) and (S)-2-((S)-7-(tert-butoxycarbonyl)-2,7-diazaspiro[4.4]nonan-2-yl)-2-cyclopentylacetic acid (518.36 mg, 1.470 mmol) in DCM (16 mL) were added DIPEA (3.02 mL, 17.326 mmol), HOBt (1324.77 mg, 9.800 mmol) and EDCI⋅HCl (913.20 mg, 5.880 mmol) at 0° C. The resulting mixture was stirred for 2 h. The mixture was diluted with DCM (30 mL). The combined organic layers were washed with H₂O (3×30 mL), dried over anhydrous Na₂SO₄. After filtration, the filtrate was concentrated under reduced pressure. The residue was purified by prep-TLC (6% MeOH/DCM) to afford the product (600 mg, 47% yield) as a solid. LCMS (ESI) m/z [M+H] calcd for C₆₄H₈₆F₃N₉O₇: 1150.67; found: 1151.1

### Step 2: Synthesis of (2S)-2-cyclopentyl-N-((6³S,4S)-1²-(2-((S)-1-methoxyethyl)-5-((R)-octahydro-2H-pyrido[1,2-a]pyrazin-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

To a stirred mixture of tert-butyl (5S)-7-((1 S)-1-cyclopentyl-2-(((6³S,4S)-1²-(2-((S)-1-methoxyethyl)-5-((R)-octahydro-2H-pyrido[1,2-a]pyrazin-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane- 4-yl)amino)-2-oxoethyl)-2,7-diazaspiro[4.4]nonane-2-carboxylate (600 mg, 0.522 mmol) in DCM (6 mL) was added and TFA (6 mL) at 0° C. The resulting mixture was stirred for 1 h. The resulting mixture was concentrated under reduced pressure. The residue was dissolved in DCM (5 mL). The residue was basified to pH 8 with sat. aq. NaHCO₃. The aqueous layer was extracted with DCM (3×30 mL), washed with brine (30 mL), dried over anhydrous Na₂SO₄. After filtration, the filtrate was concentrated under reduced pressure to afford the product (580 mg, 95% yield) as a solid. LCMS (ESI) m/z [M+H] calcd for C₅₉H₇₈F₃N₉O₅: 1050.62; found: 1051.0

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1²-(2-((S)-1-methoxyethyl)-5-((R)-octahydro-2H-pyrido[1,2-a]pyrazin-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a stirred mixture of (2S)-2-cyclopentyl-N-((6³S,4S)-1²-(2-((S)-1-methoxyethyl)-5-((R)-octahydro-2H-pyrido[1,2-a]pyrazin-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (580 mg, 0.552 mmol) and lithium (2R,3R)-1-((R)-tert-butylsulfinyl)-3-cyclopropylaziridine-2-carboxylate (366.82 mg, 1.546 mmol) in DMF (8 mL) were added DIPEA (1 mL, 5.741 mmol) and COMU (307 mg) at 0° C. The resulting mixture was stirred for 1 h. The mixture was diluted with H₂O (30 mL). The aqueous layer was extracted with DCM (3×30 mL). The combined organic layers were washed with H₂O (3×30 mL), dried over anhydrous Na₂SO₄. After filtration, the filtrate was concentrated under reduced pressure. The residue was purified by prep-TLC (8% MeOH/DCM) to afford the product (540 mg, 69% yield) as a solid. LCMS (ESI) m/z [M+H] calcd for C₆₉H₉₃F₃N₁₀O₇S: 1263.70; found: 1264.5

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-12-(2-((S)-1-methoxyethyl)-5-((R)-octahydro-2H-pyrido[1,2-a]pyrazin-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a stirred mixture of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1²-(2-((S)-1-methoxyethyl)-5-((R)-octahydro-2H-pyrido[1,2-a]pyrazin-2-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (540 mg, 0.427 mmol) and Et₃SiH (745.35 mg, 6.405 mmol) in THE (12 mL) was added HI (546.63 mg, 4.270 mmol) dropwise at 0° C. The resulting mixture was stirred for 30 min. The reaction solution was added to cold solution of DIPEA (9 g) in EtOAc (100 mL). The resulting mixture were washed with brine (7×20 mL), dried over anhydrous Na₂SO₄. After filtration, the filtrate was concentrated under reduced pressure at room temperature. The residue was purified by prep-TLC (8% MeOH/DCM) to afford the product (140 mg, 27% yield) as an solid. LCMS (ESI) m/z [M+H] calcd for C₆₅H₈₅F₃N₁₀O₆: 1159.67; found: 1160.0

### Synthesis (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

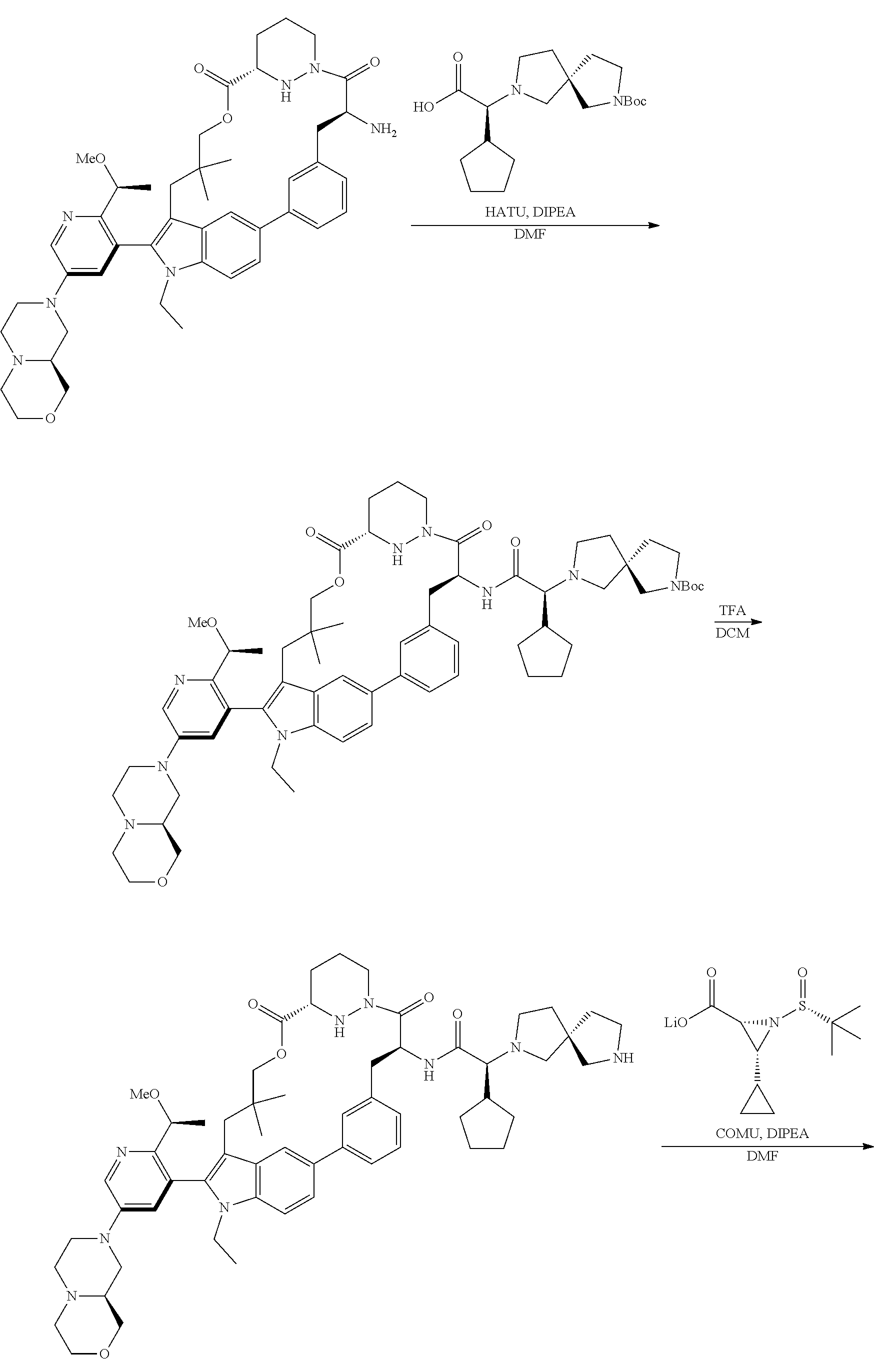

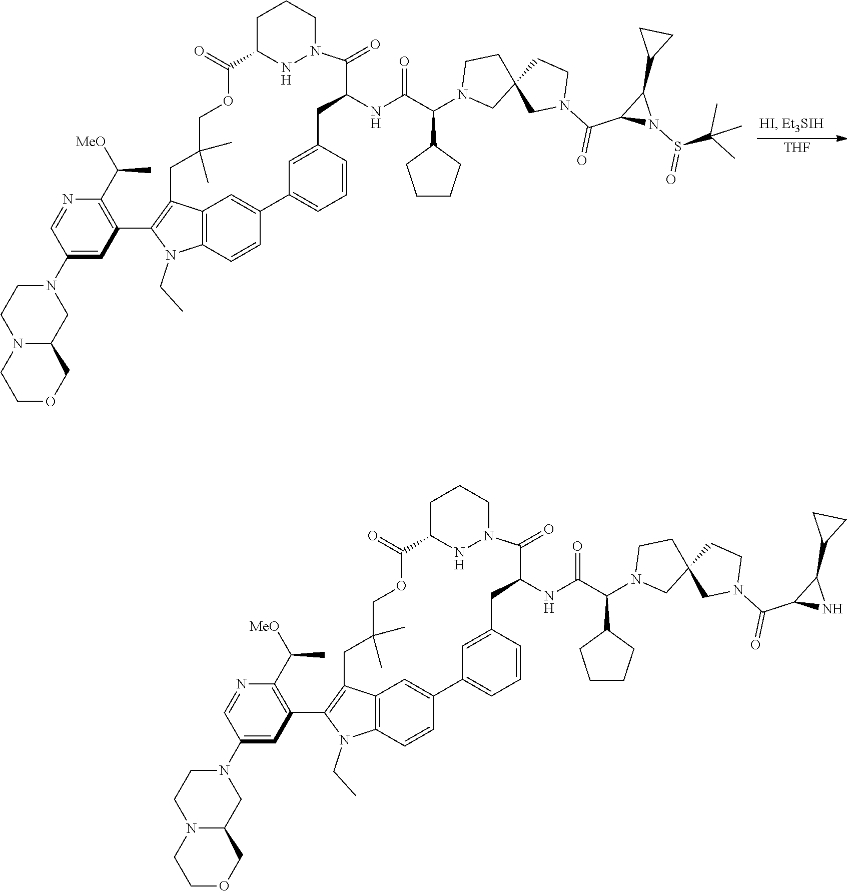

### Step 1: Synthesis of tert-butyl (5S)-7-((1 S)-1-cyclopentyl-2-(((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

To a stirred mixture of (6³S,4S)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (200 mg, 0.262 mmol) and DIPEA (1361.14 mg, 10.54 mmol) in MeCN (3 mL) were added (S)-2-((S)-7-(tert-butoxycarbonyl)-2,7-diazaspiro[4.4]nonan-2-yl)-2-cyclopentylacetic acid (157.08 mg, 0.446 mmol) and HATU (149.31 mg, 0.393 mmol) in portions at 0° C. under a nitrogen atmosphere. After 2 h, the resulting mixture was extracted with EtOAc (3×50 mL). The combined organic layers were washed with brine (3×40 mL), dried over anhydrous Na₂SO₄. After filtration, the filtrate was concentrated under reduced pressure. The residue was purified by prep-TLC (10% MeOH/DCM) to afford the product (260 mg, 90% yield) as a solid.

LCMS (ESI) m/z [M+H] calcd for C₆₃H₈₇N₉O₈: 1098.68; found: 1098.9

### Step 2: Synthesis of (2S)-2-cyclopentyl-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

To a stirred mixture of tert-butyl (5S)-7-((1 S)-1-cyclopentyl-2-(((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (250 mg, 0.246 mmol) in DCM (2.6 mL) was added TFA (1.3 mL) dropwise at 0° C. under a nitrogen atmosphere. After 1 h, the mixture was basified to pH 8 with sat. aq. Na₂CO₃. The resulting mixture was extracted with DCM (3×40 mL). The combined organic layers were washed with brine (3×30 mL), dried over anhydrous Na₂SO₄. After filtration, the filtrate was concentrated under reduced pressure to afford the product (180 mg), which was used in the next step directly without further purification. LCMS (ESI) m/z [M+H] calcd for C₅₈H₇₉N₉O₆: 998.62; found: 998.7

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a stirred mixture of (2S)-2-cyclopentyl-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (160 mg, 0.160 mmol) and DIPEA (833.51 mg, 6.438 mmol) in MeCN (2 mL) were added lithium (2R,3R)-1-((R)-tert-butylsulfinyl)-3-cyclopropylaziridine-2-carboxylate (52.3 mg, 0.220 mmol) and COMU (102.96 mg, 0.240 mmol) in portions at 0° C. under a nitrogen atmosphere. After 2 h, the resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by prep-TLC (10% MeOH/DCM) to afford the product (100 mg, 51% yield) as a solid. LCMS (ESI) m/z [M+H] calcd for C₆₈H₉₄N₁₀O₈S: 1211.71; found: 1211.3

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

Into a 40 mL vial were added (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (75 mg, 0.062 mmol) and THE (2 mL) at 0° C. Then triethylsilane (71.98 mg, 0.620 mmol) and HI (79.18 mg, 0.620 mmol) was added dropwise. After 30 min, the mixture was added into a mixture of DIPEA (80.5 mg, 0.620 mmol) and EtOAc (4 mL) at 0° C. The combined organic layers were washed with brine (2×30 mL), dried over anhydrous Na₂SO₄. After filtration, the filtrate was concentrated under reduced pressure. The residue was purified by prep-TLC (3% MeOH/DCM) to afford the product (3.2 mg, 5% yield) as a solid. LCMS (ESI) m/z; calcd for C₆₄H₈₆N₁₀O₇: 1107.68; found: 1107.9

### Synthesis of (2S)-2-((S)-7-((2R,3R)-3-cyclopropylaziridine-2-carbonyl)-2,7-diazaspiro[4.4]nonan-2-yl)-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

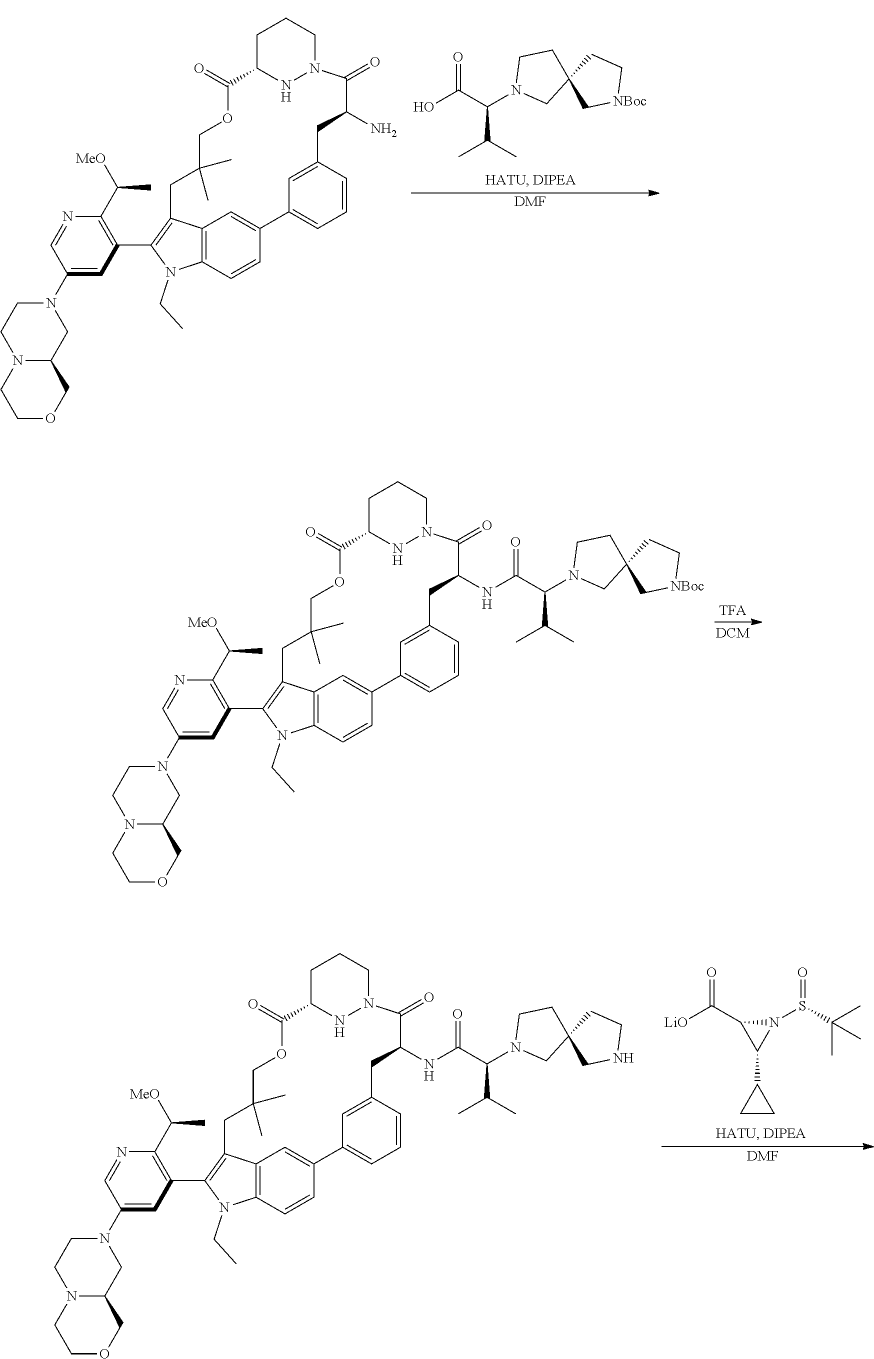

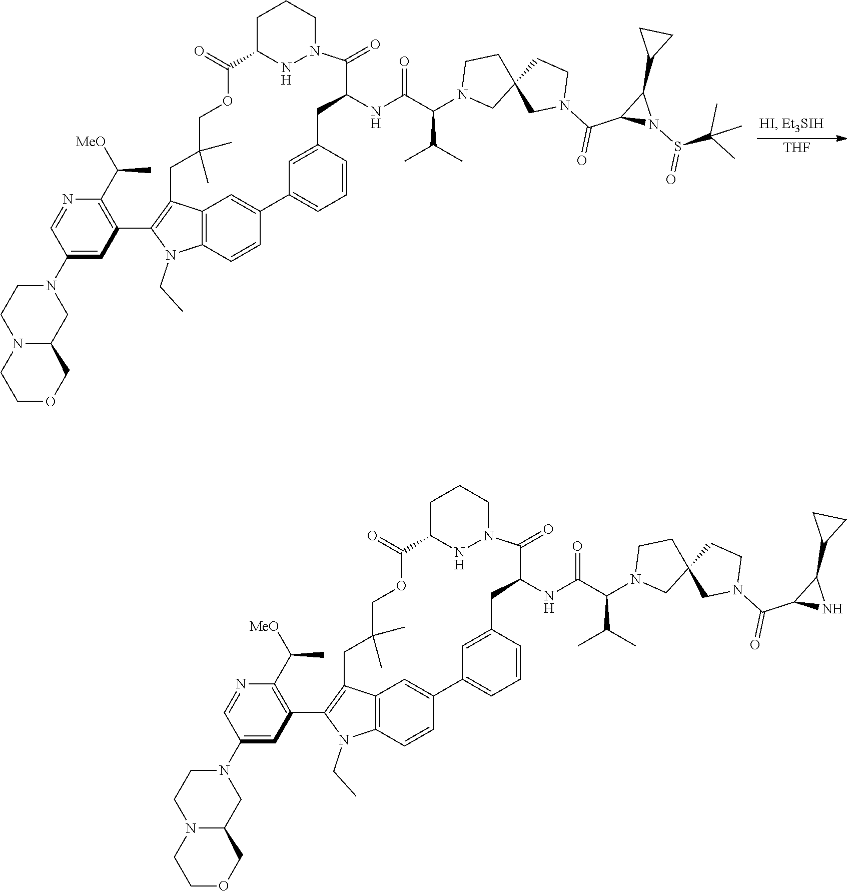

### Step 1: Synthesis of (6³S,4S)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione

To a stirred solution of tert-butyl ((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (700 mg, 0.810 mmol) in DCM (7 mL) was added TFA (3 mL) dropwise at 0° C. The mixture was basified to pH 8 with sat. aq. NaHCO₃. The resulting mixture was extracted with EtOAc (3×30 mL). The combined organic layers were washed with brine (3×50 mL), dried over anhydrous Na₂SO₄. After filtration, the filtrate was concentrated under reduced pressure to afford the product (600 mg, 96% yield) as a solid.

LCMS (ESI) m/z [M+H] Calcud for C₄₄H₅₇N₇O₅: 764.45; found: 764.6

### Step 2: Synthesis of tert-butyl (5S)-7-((2S)-1-(((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-2,7-diazaspiro[4.4]nonane-2-carboxylate

To a stirred solution of (6³S,4S)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (600 mg, 0.785 mmol) and (S)-2-((S)-7-(tert-butoxycarbonyl)-2,7-diazaspiro[4.4]nonan-2-yl)-3-methylbutanoic acid (384.55 mg, 1.177 mmol) in DMF (4 mL) was added DIPEA (4060.08 mg, 31.400 mmol) and HATU (447.92 mg, 1.177 mmol) in portions at 0° C. The resulting mixture was stirred for 1 h at room temperature then extracted with EtOAc (3×30 mL). The combined organic layers were washed with brine (3×30 mL), dried over anhydrous Na₂SO₄. After filtration, the filtrate was concentrated under reduced pressure. The residue was purified by prep-TLC (8% MeOH/DCM) to afford the product (600 mg, 71% yield) as a solid. LCMS (ESI) m/z [M+H] calcd for C₆₁H₈₅N₉O₈: 1072.66; found: 1072.9

### Step 3: Synthesis of (2S)—N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-((S)-2,7-diazaspiro[4.4]nonan-2-yl)butanamide

To a stirred solution of tert-butyl (5S)-7-((2S)-1-(((63S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-2,7-diazaspiro[4.4]nonane-2-carboxylate (600 mg, 0.559 mmol) in DCM (6 mL) was added TFA (3 mL) dropwise at 0° C. The resulting mixture was stirred for 1 h at room temperature. The mixture was basified to pH 8 with sat. aq. NaHCO₃. The resulting mixture was extracted with EtOAc (3×30 mL). The combined organic layers were washed with brine (3×30 mL), dried over anhydrous Na₂SO₄. After filtration, the filtrate was concentrated under reduced pressure to afford the product (450 mg, 82% yield) as a solid. LCMS (ESI) m/z [M+H] calcd for C₅₆H₇₇N₉O₆: 972.61; found: 972.8

### Step 4: Synthesis of (2S)-2-((S)-7-((2R,3R)-1-((R)-tert-butylsulfinyl)-3-cyclopropylaziridine-2-carbonyl)-2,7-diazaspiro[4.4]nonan-2-yl)-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)-3-methylbutanamide

To a stirred solution of (2S)—N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methyl-2-((S)-2,7-diazaspiro[4.4]nonan-2-yl)butanamide (439 mg, 0.452 mmol) and lithium (2R,3R)-3-cyclopropyl-1-[(R)-2-methylpropane-2-sulfinyl]aziridine-2-carboxylate (156.66 mg, 0.678 mmol) in DMF (4 mL) were added DIPEA (2334.19 mg, 18.080 mmol) and HATU (257.52 mg, 0.678 mmol) in portions at 0° C. under a nitrogen atmosphere. The resulting mixture was stirred for 1 h at room temperature. The resulting mixture was extracted with EtOAc (3×50 mL). The combined organic layers were washed with brine (3×30 mL), dried over anhydrous Na₂SO₄. After filtration, the filtrate was concentrated under reduced pressure. The residue was purified by prep-TLC (8% MeOH/DCM) to afford the product (300 mg, 56% yield) as a solid.

LCMS (ESI) m/z [M+H] calcd for C₆₆H₉₂N₁₀O₈S: 1185.69; found: 1186.0

### Step 5: Synthesis of (2S)-2-((S)-7-((2R,3R)-3-cyclopropylaziridine-2-carbonyl)-2,7-diazaspiro[4.4]nonan-2-yl)-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide

To a stirred solution of (2S)-2-((S)-7-((2R,3R)-1-((R)-tert-butylsulfinyl)-3-cyclopropylaziridine-2-carbonyl)-2,7-diazaspiro[4.4]nonan-2-yl)-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-3-methylbutanamide (425 mg, 0.358 mmol) in THE was added Et₃SiH (416.82 mg, 3.580 mmol) and HI (458.53 mg, 3.580 mmol) dropwise at 0° C. under a nitrogen atmosphere. The resulting mixture was stirred for 20 min at 0° C. The resulting mixture was diluted with EtOAc (200 mL) and DIPEA (1.85 g, 14.32 mmol) at 0° C. The resulting mixture was washed with brine (3×300 mL) dried over anhydrous Na₂SO₄. After filtration, the filtrate was concentrated under reduced pressure. The residue was purified by prep-TLC (8% MeOH/DCM) to afford the product (100 mg, 25% yield) as a solid. LCMS (ESI) m/z [M+H] calcd for C₆₂H₈₄N₁₀O₇: 1081.66; found: 1082.0

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

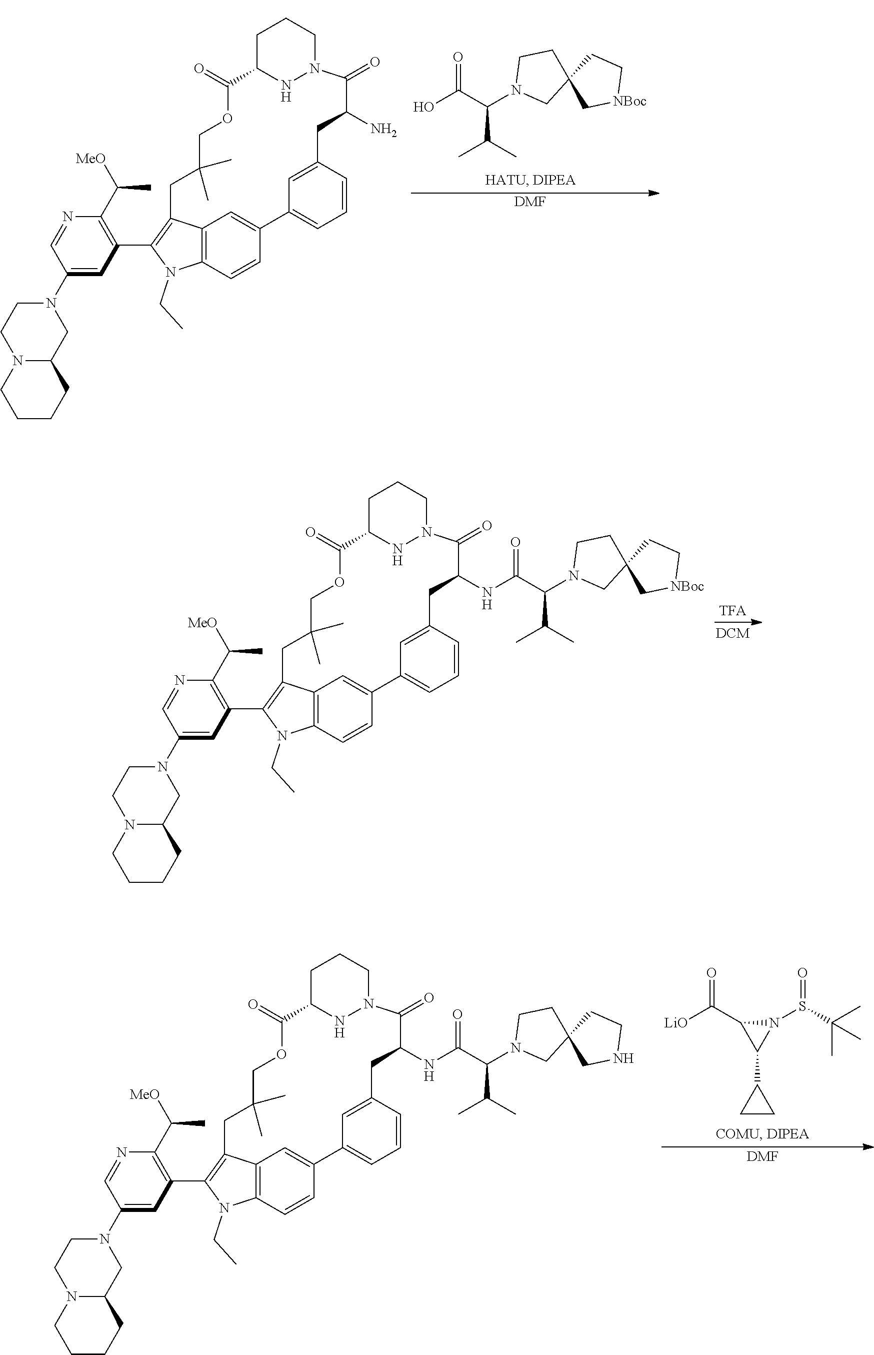

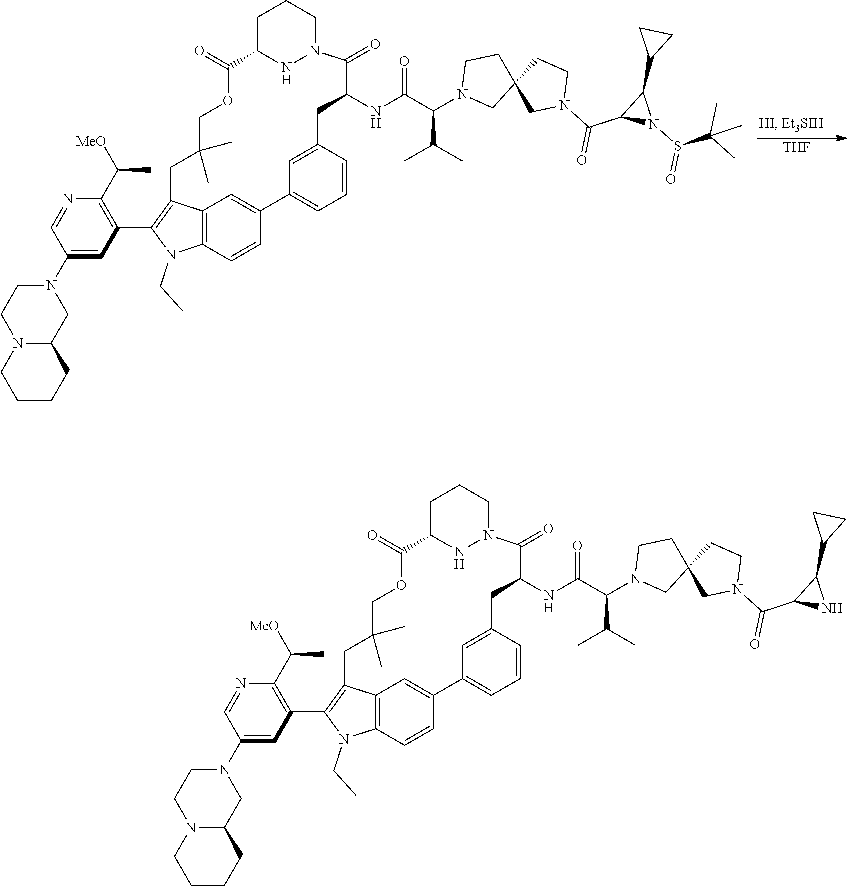

### Step 1: Synthesis of tert-butyl (5S)-7-((1S)-1-cyclopentyl-2-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,64,65,66-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

To a stirred mixture of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-1 (5,3)-indola-6(1,3)-pyridazina-2(1,3)-benzenacycloundecaphane-5,7-dione (300 mg, 0.394 mmol) and DIPEA (2035 mg, 15.760 mmol) in MeCN (5 mL) was added (S)-2-((S)-7-(tert-butoxycarbonyl)-2,7-diazaspiro[4.4]nonan-2-yl)-2-cyclopentylacetic acid (277.53 mg, 0.788 mmol) and HATU (224.54 mg) in portions at 0° C. under a nitrogen atmosphere. After 2 h, the resulting mixture was concentrated under reduced pressure. The residue was purified by prep-TLC (10% MeOH/DCM) to the product (270 mg, 62% yield) as a solid. LCMS (ESI) m/z [M+H] calcd for C₆₄H₈₉N₉O₇: 1096.70; found: 1096.4

### Step 2: Synthesis of (2S)-2-cyclopentyl-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

To a stirred mixture of tert-butyl (5S)-7-((1 S)-1-cyclopentyl-2-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (270 mg, 0.246 mmol) in DCM (3 mL) was added TFA (1.5 mL) dropwise at 0° C. After 1 h, the mixture was basified to pH 8 with sat. aq. NaHCO₃. The resulting mixture was extracted with DCM (3×50 mL). The combined organic layers were washed with brine (3×30 mL), dried over anhydrous Na₂SO₄. After filtration, the filtrate was concentrated under reduced pressure to afford the product (180 mg, crude). LCMS (ESI) m/z [M+H] calcd for C₅₉H₈₁N₉O₅: 996.64; found: 996.6

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a stirred mixture of (2S)-2-cyclopentyl-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (180 mg, 0.181 mmol) and DIPEA (937.69 mg, 7.269 mmol) in MeCN (2 mL) were added lithium (2R,3R)-1-((R)-tert-butylsulfinyl)-3-cyclopropylaziridine-2-carboxylate (47.5 mg, 0.200 mmol) and COMU (64.3 mg, 0.150 mmol) in portions at 0° C. under a nitrogen atmosphere. After 2 h, the resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by prep-TLC (10% MeOH/DCM) to afford the product (100 mg, 45% yield) as a solid. LCMS (ESI) m/z [M+H] calcd for C₆₉H₉₆N₁₀O₇S: 1209.73; found: 1210.0

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

Into a 40 mL vial were added (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (75 mg, 0.062 mmol) and THE (2 mL) at 0° C. Then triethylsilane (72.09 mg, 0.620 mmol) and HI (79.31 mg, 0.620 mmol) was added dropwise. After 30 min, the above solution was added into a mixture of DIPEA (225 mg, 0.620 mmol) and EtOAc (5 mL). The combined organic layers were washed with brine (3×20 mL), dried over anhydrous Na₂SO₄. After filtration, the filtrate was concentrated under reduced pressure. The residue was purified by prep-TLC (4% MeOH/DCM) to afford the product (1.6 mg, 3% yield) as a solid. LCMS (ESI) m/z [M+H] calcd for C₆₅H₈₈N₁₀O₆: 1105.70; found: 1106.1

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

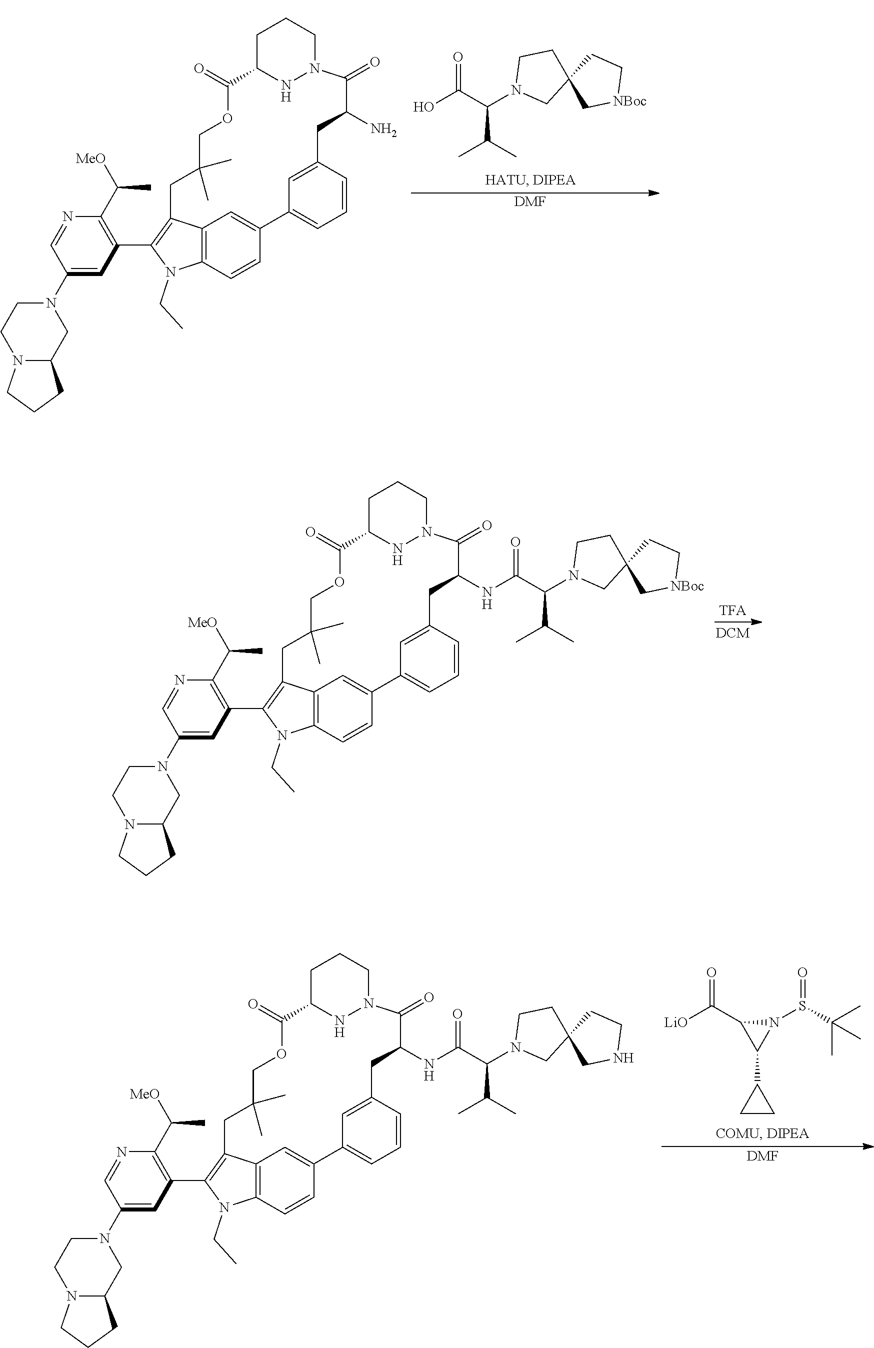

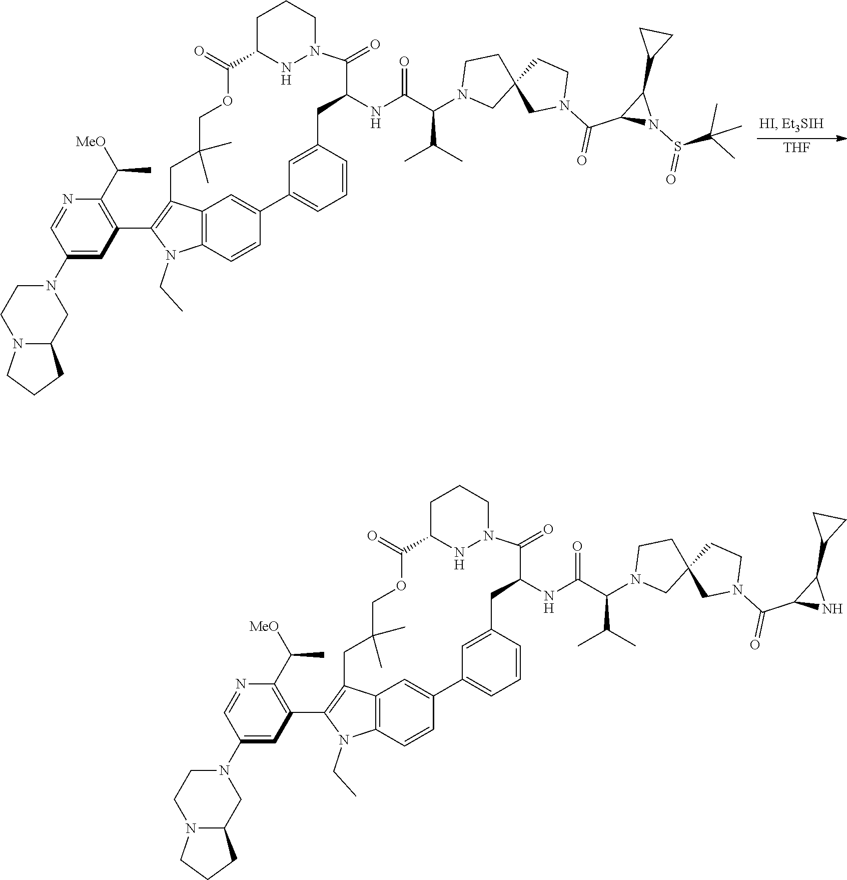

### Step 1: Synthesis of tert-butyl (5S)-7-((1 S)-1-cyclopentyl-2-(((6³S,4S)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

Into a 100 mL round-bottom flask was added (6³S,4S)-4-amino-1′-ethyl-1²-(5-((R)-hexahydropyrrolo[1,2-a]pyrazin-2(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (226 mg, 0.642 mmol), DMF (8 mL), DIPEA (345 mg, 2.675 mmol), HATU (610 mg, 1.605 mmol), at 0° C. The resulting mixture was diluted with H₂O (100 mL). The resulting mixture was extracted with EtOAc (2×100 mL). The combined organic layers were washed with brine (2×100 mL), dried over anhydrous Na₂SO₄. After filtration, the filtrate was concentrated under reduced pressure. The residue was purified by silica gel column chromatography (10% MeOH/DCM) to afford the product (400 mg, 69% yield) as a solid. LCMS (ESI) m/z [M+H] calcd for C₆₃H₈₇N₉O₇: 1082.68; found: 1082.5

### Step 2: Synthesis of (2S)-2-cyclopentyl-N-((6³S,4S)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

Into a 40 mL vial was added tert-butyl (5S)-7-((1S)-1-cyclopentyl-2-(((6³S,4S)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (400 mg, 0.370 mmol) and DCM (8 mL), TFA (4 mL) at 0° C. The resulting mixture was stirred for 3 h at room temperature then concentrated under reduced pressure to afford the product (300 mg, 82% yield) as a solid. LCMS (ESI) m/z [M+H] calcd for C₅₈H₇₉N₉O₅: 982.63; found: 982.8

### Step 3: Synthesis of (2S)-2-((5S)-7-((2R,3R)-1-(tert-butylsulfinyl)-3˜cyclopropylaziridine-2-carbonyl)-2,7-diazaspio[4,4]nonan-2-yl)-2-cyclopentyl-N-((6³S,4S)-1¹-ethyl-1²(5((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁵-hexahydro-1¹H-8-oxa-1(5,3)-indola-6(1,3)-pyridazina-2(1,3)-benzenacycloundecaphane-4-yl)acetamide

Into a 50 mL round-bottom flask was added (2S)-2-cyclopentyl-N-((6³S,4S)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (300 mg, 0.305 mmol) and (2R,3R)-3-cyclopropyl-1-[(R)-2-methylpropane-2-sulfinyl]aziridine-2-carboxylic acid (141 mg, 0.610 mmol), COMU (156.85 mg, 0.366 mmol), DIPEA (394 mg, 3.050 mmol), and DMF (6 mL) at 0° C. The resulting mixture was stirred for 3 h at 0° C. then diluted with H₂O (100 mL). The resulting mixture was extracted with EtOAc (2×100 mL). The combined organic layers were washed with brine (2×100 mL), dried over anhydrous Na₂SO₄. After filtration, the filtrate was concentrated under reduced pressure. The residue was purified by silica gel column chromatography (10% MeOH/DCM) to afford the product (200 mg, 54% yield) as a solid. LCMS (ESI) m/z [M+H] calcd for C₆₈H₉₄N₁₀O₇S: 1195.71; found: 1195.6

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

Into a 40 mL vial was added (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((63S,4S)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (200 mg, 0.167 mmol) THE (4 mL), and Et₃SiH (155.47 mg, 1.336 mmol) at 0° C. To the above mixture was added HI (171.03 mg, 1.336 mmol) dropwise at 0° C. The resulting mixture was stirred for 20 min. The resulting mixture was diluted with DIPEA (1 mL) in EtOAc (40 mL) at 0° C. The resulting mixture was washed with brine (2×40 mL). The resulting mixture was concentrated under reduced pressure. The residue was purified by prep-TLC (7% MeOH/DCM) to afford the product (20 mg, 10% yield) as a solid. LCMS (ESI) m/z [M+H] calcd for C₆₄H₈₆N₁₀O₆: 1091.68; found: 1092.1

### Synthesis of (2S)-2-cyclopentyl-2-((S)-7-((2R,3R)-3-cyclopropylaziridine-2-carbonyl)-2,7-diazaspiro[4.4]nonan-2-yl)-N-((2²S,6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide

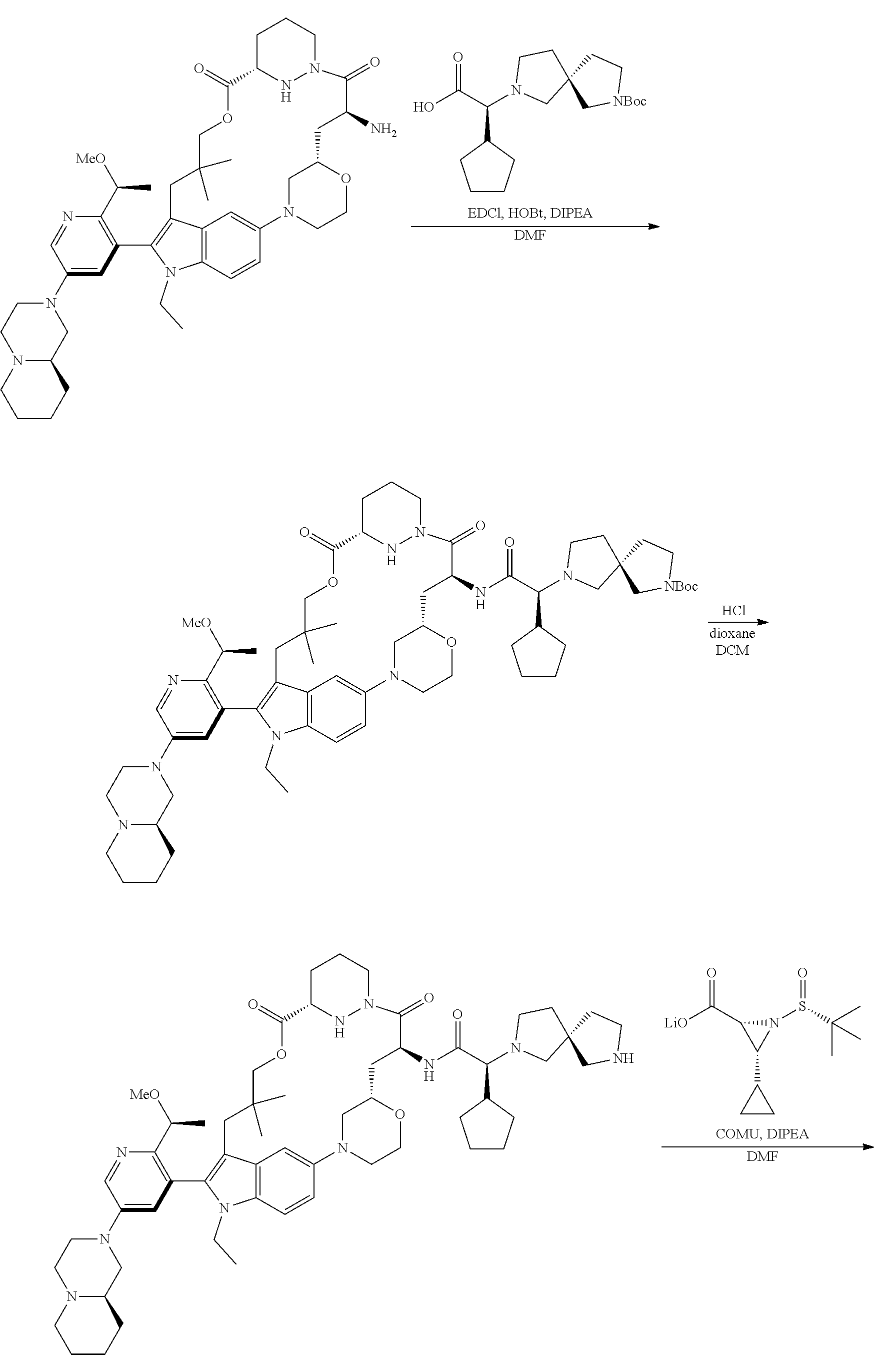

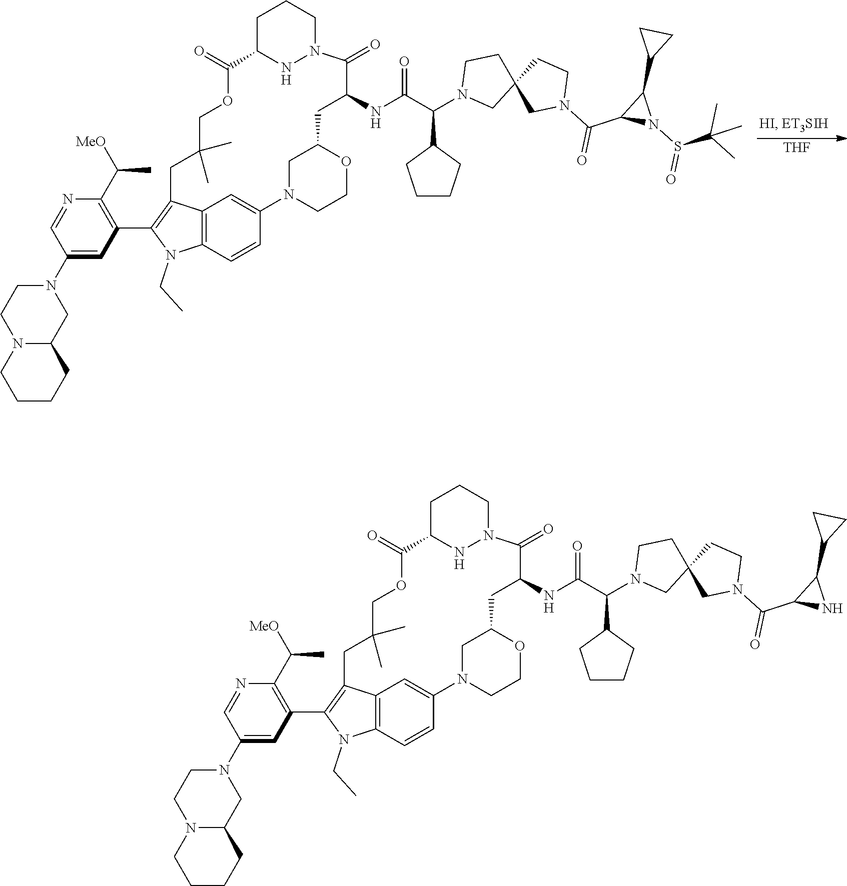

### Step 1: Synthesis of tert-butyl (5S)-7-((1 S)-1-cyclopentyl-2-(((2²S,6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-morpholina-1 (5,3)-indola-6(1,3)-pyridazinacycloundecaphane-4-yl)amino)-2-oxoethyl)-2,7-diazaspiro[4.4]nonane-2-carboxylate

To a stirred solution of (2²S,6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (800 mg, 1.038 mmol) and (S)-[(5S)-7-(tert-butoxycarbonyl)-2,7-diazaspiro[4.4]nonan-2-yl](cyclopentyl)acetic acid (438.87 mg, 1.246 mmol) in DMF (10 mL) was added DIPEA (2.71 mL, 15.57 mmol) and EDCI (1610.76 mg, 10.38 mmol) in portions at 0° C. under a nitrogen atmosphere. The resulting mixture was stirred overnight at room temperature. The reaction was quenched by the addition of H₂O (100 mL) at room temperature then extracted with EtOAc (3×100 mL). The combined organic layers were washed with brine (100 mL), dried over anhydrous Na₂SO₄. After filtration, the filtrate was concentrated under reduced pressure. The residue was purified by prep-TLC (10% MeOH/DCM) to afford the product (830 mg, 72% yield) as a solid.

LCMS (ESI) m/z [M+H] calcd for C₆₂H₉₂N₁₀O₈: 1105.72; found: 1106.0

### Step 2: Synthesis of (2S)-2-cyclopentyl-N-((2²S,6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-2-((S)-2,7-diazaspiro[4.4]nonan-2-yl)acetamide

To a stirred solution of tert-butyl (5S)-7-((1 S)-1-cyclopentyl-2-(((2²S,6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-2-oxoethyl)-2,7-diazaspiro[4.4]nonane-2-carboxylate (800 mg, 0.724 mmol) in DCM (5 mL) was added HCl (4M in dioxane, 5 mL) dropwise at 0° C. The resulting mixture was stirred for 2 h at room temperature under a nitrogen atmosphere. The resulting mixture was concentrated under reduced pressure to afford (830 mg, crude) as a solid. LCMS (ESI) m/z [M+H] calcd for C₅₇H₈₄N₁₀O₆: 1005.67; found: 1005.9

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((2²S,6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide

To a stirred solution of (2S)-2-cyclopentyl-N-((2²S,6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-2-((S)-2,7-diazaspiro[4.4]nonan-2-yl)acetamide (830 mg, 0.826 mmol) and lithium (2R,3R)-3-cyclopropyl-1-[(R)-2-methylpropane-2-sulfinyl]aziridine-2-carboxylate (293.79 mg, 1.239 mmol) in DMF (10 mL) was added DIPEA (4267.99 mg, 33.04 mmol) and COMU (459.67 mg, 1.074 mmol) at 0° C. under a nitrogen atmosphere. The resulting mixture was stirred for 1 h at room temperature. The reaction was quenched with H₂O (10 mL). The resulting mixture was extracted with EtOAc (3×20 mL). The combined organic layers were washed with brine (2×50 mL), dried over anhydrous Na₂SO₄. After filtration, the filtrate was concentrated under reduced pressure. The residue was purified by prep-TLC (7% MeOH/DCM) to afford the product (760 mg, 75% yield) as a solid. LCMS (ESI) m/z [M+H] calcd for C₆₇H₉₉N₁₁O₈S: 1218.75; found: 1219.0

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((2²S,6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-morpholina-1 (5,3)-indola-6(1,3)-pyridazinacycloundecaphane-4-yl)acetamide

To a stirred solution of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((2²S,6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide (600 mg, 0.492 mmol) in THE (10 mL) was added triethylsilane (572.49 mg, 4.92 mmol) and HI (629.77 mg, 4.92 mmol) dropwise at 0° C. under a nitrogen atmosphere. The resulting mixture was stirred for 30 min. The reaction was quenched by the addition of a mixture of EtOAc (200 mL) and DIPEA (3.5 mL) at 0° C. The combined organic layers were washed with brine (200 mL), dried over anhydrous Na₂SO₄. After filtration, the filtrate was concentrated under reduced pressure. The residue was purified by prep-TLC (7% MeOH/DCM) to afford the product (155 mg, 28% yield) as a solid. LCMS (ESI) m/z [M+H] calcd for C₆₃H₉₁N₁₁O₇: 1114.72; found: 1115.0

### Synthesis of (2S)-2-cyclopentyl-2-((S)-7-((2R,3R)-3-cyclopropylaziridine-2-carbonyl)-2,7-diazaspiro[4.4]nonan-2-yl)-N-((2²S,6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide

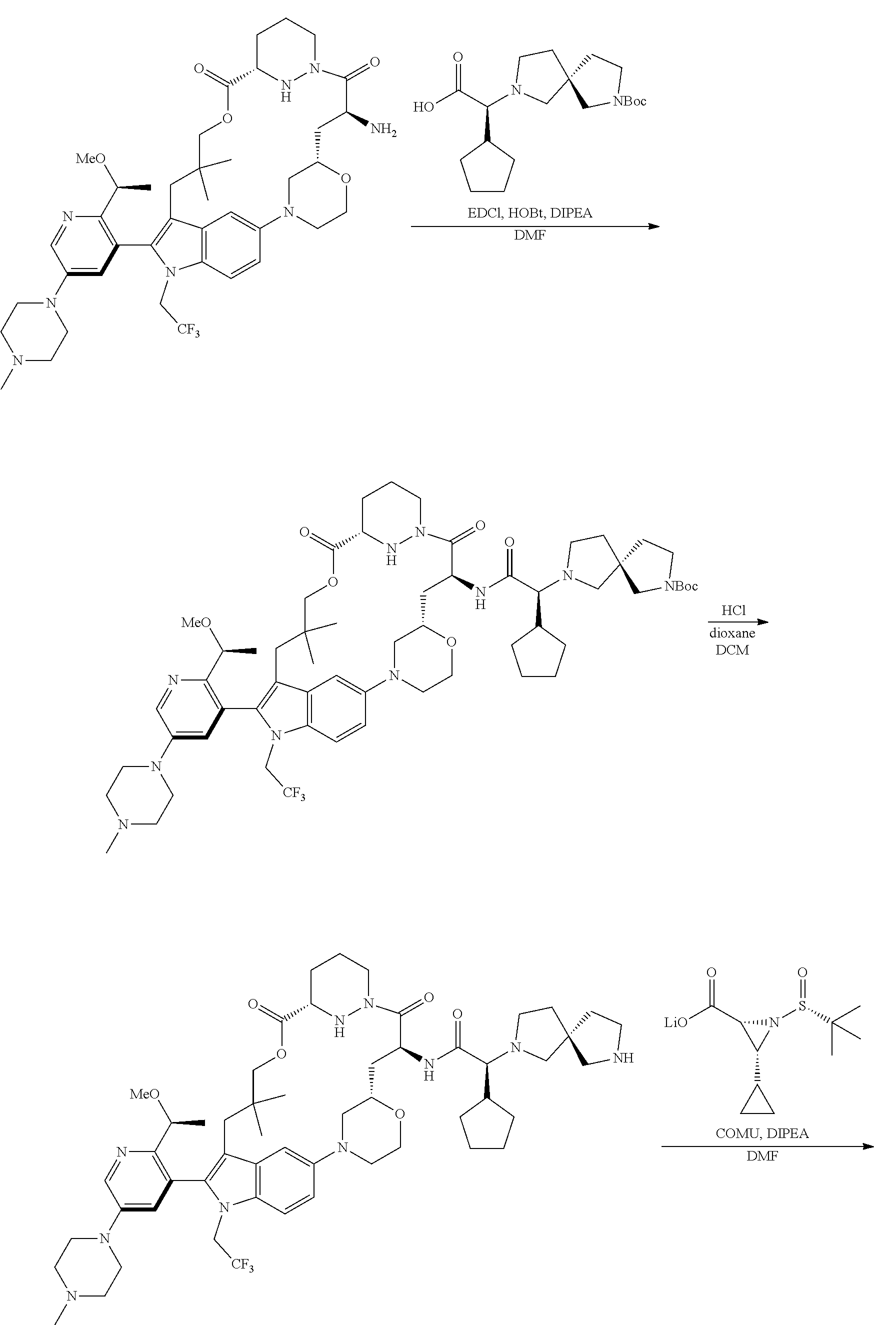

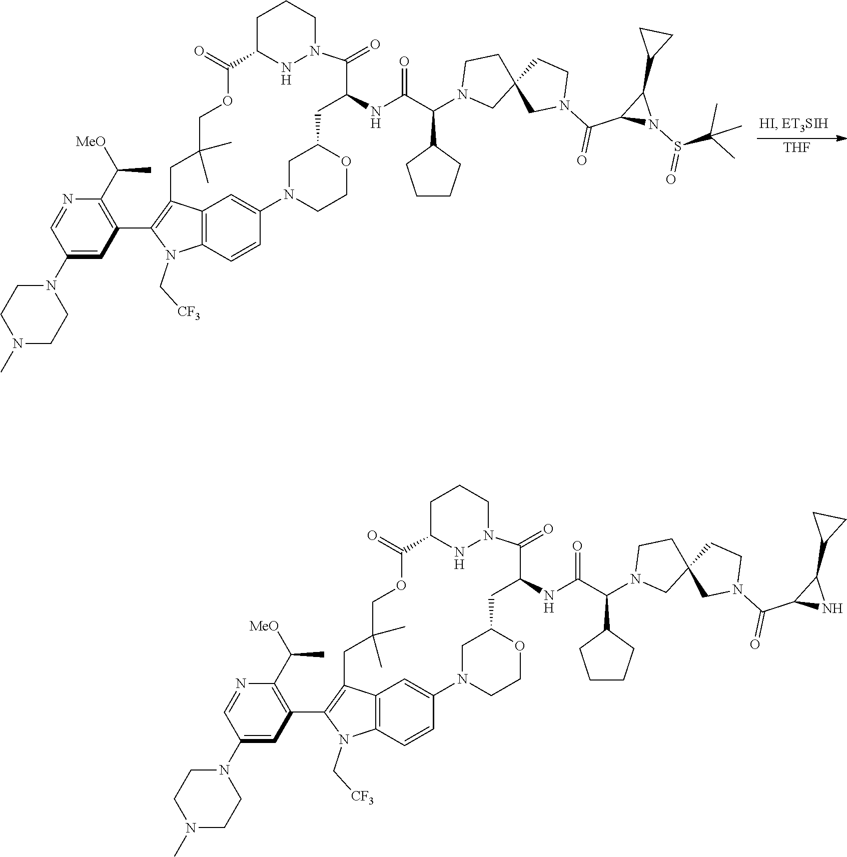

### Step 1: Synthesis of tert-butyl (5S)-7-((1S)-1-cyclopentyl-2-(((2²S,6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-2-oxoethyl)-2,7-diazaspiro[4.4]nonane-2-carboxylate

To a stirred solution of (2²S,6³S,4S)-4-amino-1²-(2-((S)-1-methoxyethyl)-5-(4-methylpiperazin-1-yl)pyridin-3-yl)-10,10-dimethyl-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-5,7-dione (200 mg, 0.255 mmol) and (S)-[(5S)-7-(tert-butoxycarbonyl)-2,7-diazaspiro[4.4]nonan-2-yl](cyclopentyl)acetic acid (134.72 mg, 0.383 mmol) in DCM (3 mL) was added DIPEA (1.78 mL, 10.219 mmol), HOBt (172.15 mg, 1.275 mmol) and EDCI (488.46 mg, 2.550 mmol) at 0° C. under a nitrogen atmosphere. The resulting mixture was stirred overnight at room temperature. The resulting mixture was concentrated under reduced pressure then diluted with EtOAc (100 mL). The organic layers were washed with brine (250 mL), dried over anhydrous Na₂SO₄. After filtration, the filtrate was concentrated under reduced pressure. The residue was purified by prep-TLC (10% MeOH/DCM) to afford the product (130 mg, 45% yield) as a solid. LCMS (ESI) m/z [M+H] calcd for C₅₉H₈₅F₃N₁₀O₈: 1119.66; found: 1120.0

### Step 2: Synthesis of (2S)-2-cyclopentyl-N-((2²S,6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-2-((S)-2,7-diazaspiro[4.4]nonan-2-yl)acetamide

To a stirred mixture of tert-butyl (5S)-7-((1 S)-1-cyclopentyl-2-(((2²S,6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)amino)-2-oxoethyl)-2,7-diazaspiro[4.4]nonane-2-carboxylate (130 mg, 0.128 mmol) in DCM (1 mL) was added HCl (4 M in dioxane) (1 mL) dropwise at 0° C. under a nitrogen atmosphere. The resulting mixture was stirred for 2 h at room temperature. The resulting mixture was concentrated under reduced pressure. This resulted in the product (130 mg, crude) as a solid. LCMS (ESI) m/z [M+H] calcd for C₅₄H₇₇F₃N₁₀O₆: 1019.61; found: 1019.8

### Step 3: Synthesis of (2S)-2-((S)-7-((2R,3R)-1-((R)-tert-butylsulfinyl)-3-cyclopropylaziridine-2-carbonyl)-2,7-diazaspiro[4.4]nonan-2-yl)-2-cyclopentyl-N-((2²S,6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide

To a stirred solution of (2S)-2-cyclopentyl-N-((2²S,6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)-2-((S)-2,7-diazaspiro[4.4]nonan-2-yl)acetamide (140 mg, 0.137 mmol) and lithium (2R,3R)-3-cyclopropyl-1-[(R)-2-methylpropane-2-sulfinyl]aziridine-2-carboxylate (65.17 mg, 0.274 mmol) in DMF (2 mL) was added DIPEA (0.96 mL, 5.480 mmol) and COMU (76.48 mg, 0.178 mmol) dropwise at 0° C. under a nitrogen atmosphere. The resulting mixture was stirred for 1 h at room temperature. The reaction was quenched with H₂O at room temperature. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by prep-TLC (10% MeOH/DCM) to afford the product (130 mg, 76% yield) as a solid. LCMS (ESI) m/z [M+H] calcd for C₆₄H₉₂F₃N₁₁O₈S: 1232.69; found: 1232.9

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((2²S,6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide

To a stirred mixture of (2S)-2-((S)-7-((2R,3R)-1-((R)-tert-butylsulfinyl)-3-cyclopropylaziridine-2-carbonyl)-2,7-diazaspiro[4.4]nonan-2-yl)-2-cyclopentyl-N-((2²S,6³S,4S)-1²-(2-((S)-1-methoxyethyl)-5-(4-methylpiperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-2(4,2)-morpholina-1(5,3)-indola-6(1,3)-pyridazinacycloundecaphane-4-yl)acetamide (116 mg, 0.094 mmol) in THE (3 mL) was added triethylsilane (109.43 mg, 0.940 mmol) and HI (120.38 mg, 0.940 mmol) dropwise at 0° C. under a nitrogen atmosphere. The resulting mixture was stirred for 40 min at 0° C. The reaction was quenched by the addition of EtOAc (200 mL) and DIPEA (0.64 mL) at 0° C. The combined organic layers were washed with brine (100 mL), dried over anhydrous Na₂SO₄. After filtration, the filtrate was concentrated under reduced pressure. The residue was purified by prep-TLC (10% MeOH/DCM) to afford the product (45 mg, 42% yield) as a solid. LMCS(ESI) m/z [M+H] calcd for C₆₀H₈₄F₃N₁₁O₇: 1128.66; found: 1129.0

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6-hexahydro-1¹H-8-oxa-1(5,3)-indola-6(1,3)-pyridazina-2(1,3)-benzenacycloundecaphane-4-yl)acetamide

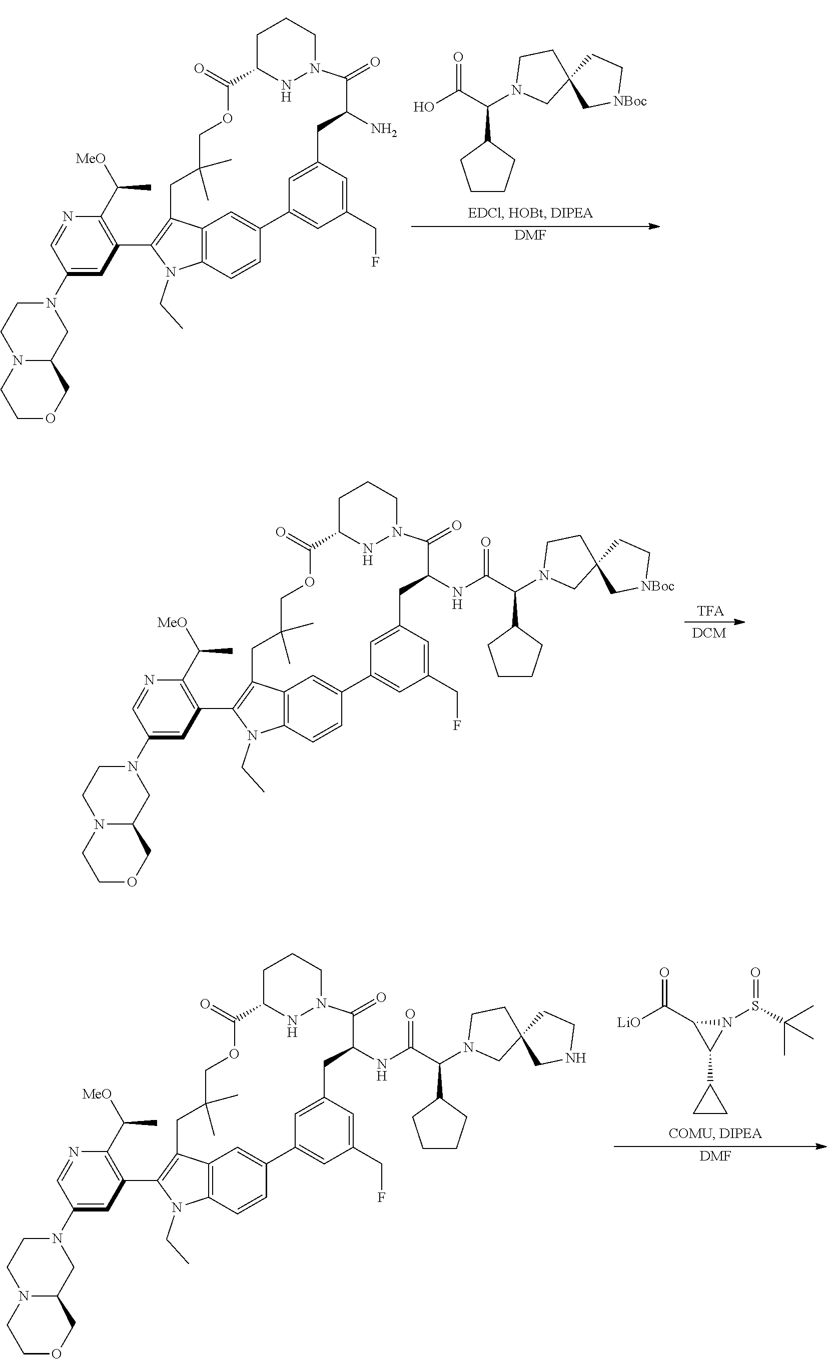

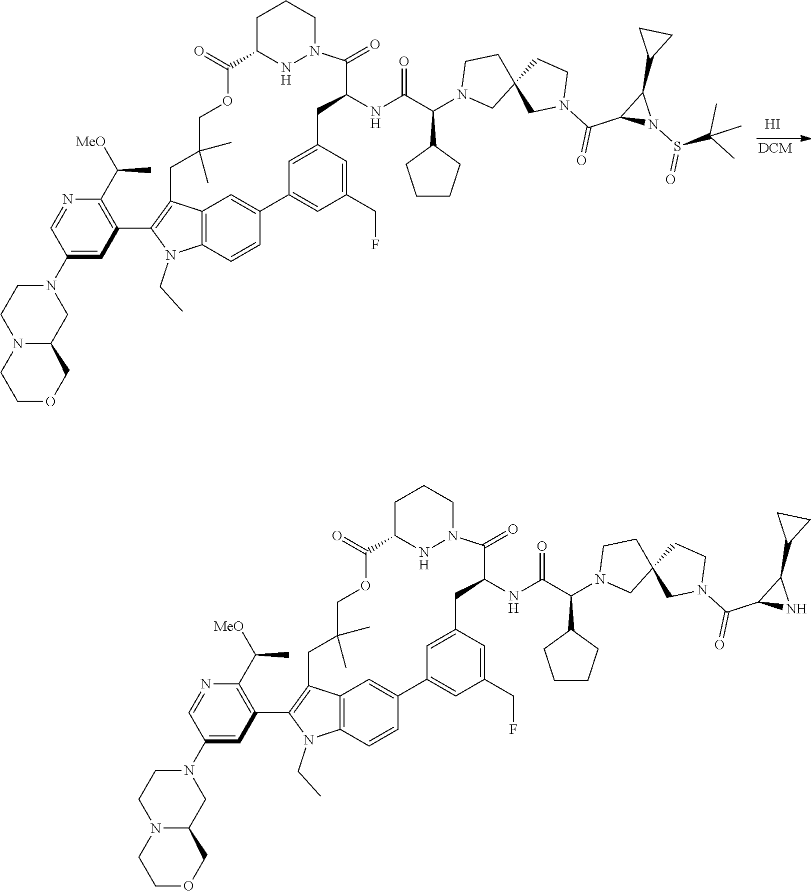

### Step 1: Synthesis of tert-butyl (5S)-7-((1 S)-1-cyclopentyl-2-(((6³S,4S)-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)-2,7-diazaspiro[4.4]nonane-2-carboxylate

To a solution of (6³S,4S)-4-amino-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (250 mg, 0.314 mmol), DIPEA (1217 mg, 9.420 mmol) and (S)-[(5S)-7-(tert-butoxycarbonyl)-2,7-diazaspiro[4.4]nonan-2-yl](cyclopentyl)acetic acid (166 mg, 0.471 mmol) in DCM (50 mL) was added HOBt (424 mg, 3.140 mmol) and EDCI (361 mg, 1.884 mmol) at 0° C. The resulting mixture was stirred for 1 h. The resulting mixture was washed with brine (5×20 mL), dried over anhydrous Na₂SO₄. After filtration, the filtrate was concentrated under reduced pressure. The residue was purified by reverse phase chromatography (0→100% MeCN/H₂O 0.1% NH₄HCO₃). To afford product (275 mg, 77% yield) as a solid. LCMS (ESI) m/z [M+H] calcd for C₆₄H₈₈FN₉O₈: 1130.68; found: 1130.2

### Step 2: Synthesis of (2S)-2-cyclopentyl-N-((6³S,4S)-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

To a solution of tert-butyl (5S)-7-((1 S)-1-cyclopentyl-2-(((6³S,4S)-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (275 mg, 0.243 mmol) in DCM (3 mL) was added TFA (1.5 mL) at 0° C. The resulting mixture was stirred for 1 h. The mixture was concentrated under reduced pressure, basified to pH 7 with sat. aq. NaHCO₃. The resulting mixture was extracted with 10% DCM/MeOH (4×100 mL). The combined organic layers were washed with brine (3×100 mL), dried over anhydrous Na₂SO₄. After filtration, the filtrate was concentrated under reduced pressure to afford the product (230 mg, 92% yield) as a solid. LCMS (ESI) m/z [M+H] calcd for C₅₉H₈₀FN₉O₆: 1030.63; found: 1030.2

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-cyclopentyl-N-((6³S,4S)-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (250 mg, 0.243 mmol), DIPEA (313 mg, 2.430 mmol) and lithium (2R,3R)-3-cyclopropyl-1-[(R)-2-methylpropane-2-sulfinyl]aziridine-2-carboxylate (173 mg, 0.729 mmol) in DMF (3 mL) was added COMU (125 mg, 0.292 mmol) in portions at 0° C. The resulting mixture was stirred for 1 h. The residue was purified by reverse phase chromatography (0→100% MeCN/H₂O 0.1% NH₄HCO₃) to afford the product (210 mg, 69% yield) as a solid. LCMS (ESI) m/z [M+H] calcd for C₆₉H₉₅FN₁₀O₈S: 1243.71; found: 1243.3

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-2⁵-(fluoromethyl)-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (600 mg, 0.482 mmol) in THE (90 mL) was added HI (370 mg, 2.892 mmol) in THE (3 mL) at 0° C. The resulting mixture was stirred for 30 min at 0° C. under a nitrogen atmosphere. The reaction was added to a solution of DIPEA (9 g) in cold EtOAc (100 mL). The resulting mixture was washed with brine (7×20 mL), dried over anhydrous Na₂SO₄. After filtration, the filtrate was concentrated under reduced pressure at room temperature. The residue was purified by prep-TLC (8% MeOH/DCM) to afford the product (121.9 mg, 20% yield) as a solid. LCMS (ESI) m/z [M+H] calcd for C₆₅H₈₇FN₁₀O₇: 1139.68; found: 1140.1

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6-hexahydro-1¹H-8-oxa-1(5,3)-indola-6(1,3)-pyridazina-2(1,3)-benzenacycloundecaphane-4-yl)acetamide

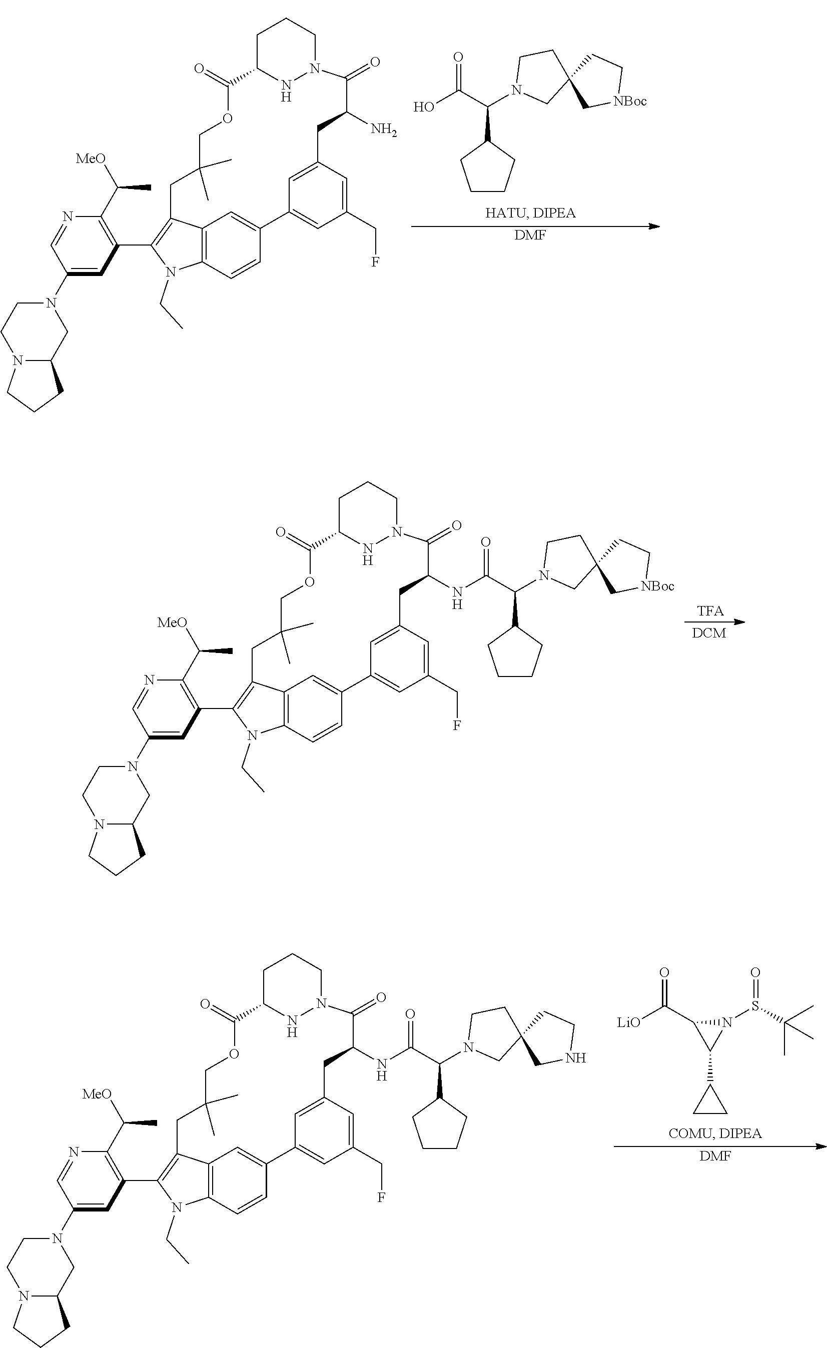

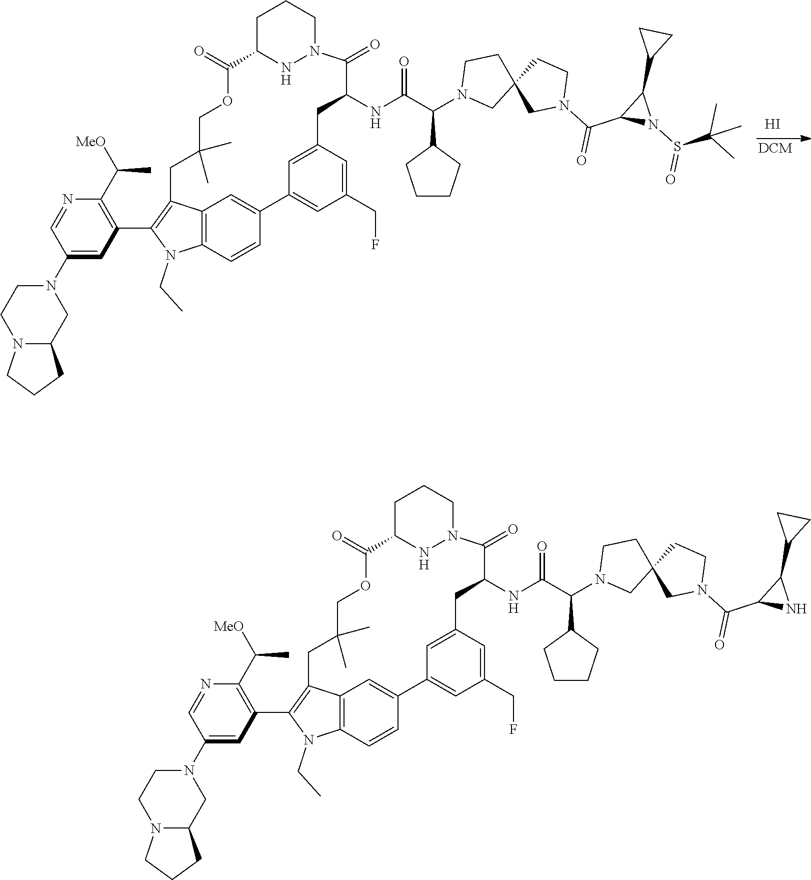

### Step 1: Synthesis of tert-butyl (5S)-7-((1 S)-1-cyclopentyl-2-(((6³S,4S)-1¹-ethyl-2⁵-(fluoromethyl)-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

To a solution of (6³S,4S)-4-amino-1¹-ethyl-1⁶-(fluoromethyl)-1²-(5-((R)-hexahydropyrrolo[1,2-a]pyrazin-2(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (600 mg, 0.769 mmol), DIPEA (994.18 mg, 7.690 mmol) and (S)-[(5S)-7-(tert-butoxycarbonyl)-2,7-diazaspiro[4.4]nonan-2-yl](cyclopentyl)acetic acid (406.70 mg, 1.153 mmol) in DMF (7 mL) was added HATU (438.73 mg, 1.153 mmol) at 0° C. The resulting mixture was stirred for 1 h at room temperature under nitrogen atmosphere. The residue was purified by reverse phase chromatography (0→100% MeCN/H₂O 0.1% NH₄HCO₃) to afford the product (465 mg, 54%) as a solid. LCMS (ESI) m/z [M+H] calcd for C₆₄H₈₈FN₉O₇: 1114.69; found: 1114.5

### Step 2: Synthesis of (2S)-2-cyclopentyl-N-((6³S,4S)-1¹-ethyl-2⁵-(fluoromethyl)-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

To a solution of tert-butyl (5S)-7-((1 S)-1-cyclopentyl-2-(((6³S,4S)-1¹-ethyl-2⁵-(fluoromethyl)-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (460 mg, 0.413 mmol) in DCM (6 mL) was added TFA (3 mL) at 0° C. The resulting mixture was stirred for 1 h at room temperature under a nitrogen atmosphere. The mixture was basified to pH 7˜8 with sat. aq. NaHCO₃. The resulting mixture was extracted with EtOAc (3×20 mL). The combined organic layers were washed with brine (3×20 mL), dried over anhydrous Na₂SO₄. After filtration, the filtrate was concentrated under reduced pressure to afford the product (340 mg, crude) as a solid. The crude product was used in the next step directly without further purification. LCMS (ESI) m/z [M+H] calcd for C₅₉H₈₀FN₉O₅: 1014.63; found: 1014.6

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-2⁵-(fluoromethyl)-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-cyclopentyl-N-((6³S,4S)-1¹-ethyl-2⁵-(fluoromethyl)-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (500 mg, 0.486 mmol), DIPEA (628.39 mg, 4.860 mmol), and lithium (2R,3R)-3-cyclopropyl-1-[(R)-2-methylpropane-2-sulfinyl]aziridine-2-carboxylate (346.04 mg, 1.458 mmol) in DMF was added COMU (249.72 mg, 0.583 mmol) at 0° C. The resulting mixture was stirred for 1 h at room temperature under nitrogen atmosphere. The residue was purified by reverse phase chromatography (0→100% MeCN/H₂O 0.1% NH₄HCO₃). to afford the product (480 mg, 80%) as a solid. LCMS (ESI) m/z [M+H] calcd C₆₉H₉₅FN₁₀O₇S: 1227.72; found: 1227.7

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)acetamide

To a solution of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-2⁵-(fluoromethyl)-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (310 mg, 0.253 mmol) in THE (45 mL) was added a solution of HI (193.80 mg, 1.518 mmol.) in THE (0.5 mL) at 0° C. The resulting mixture was stirred for 20 min at 0° C. under a nitrogen atmosphere. The reaction was quenched by the addition of a solution of DIPEA (300 mg) in EtOAc (50 mL) at 0° C. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by prep-TLC (10% MeOH/DCM) to afford the product (94.9 mg, 32%) as a solid. LCMS (ESI) m/z [M+H] calcd for C₆₅H₈₇FN₁₀O₆: 1123.69; found: 1124.1

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

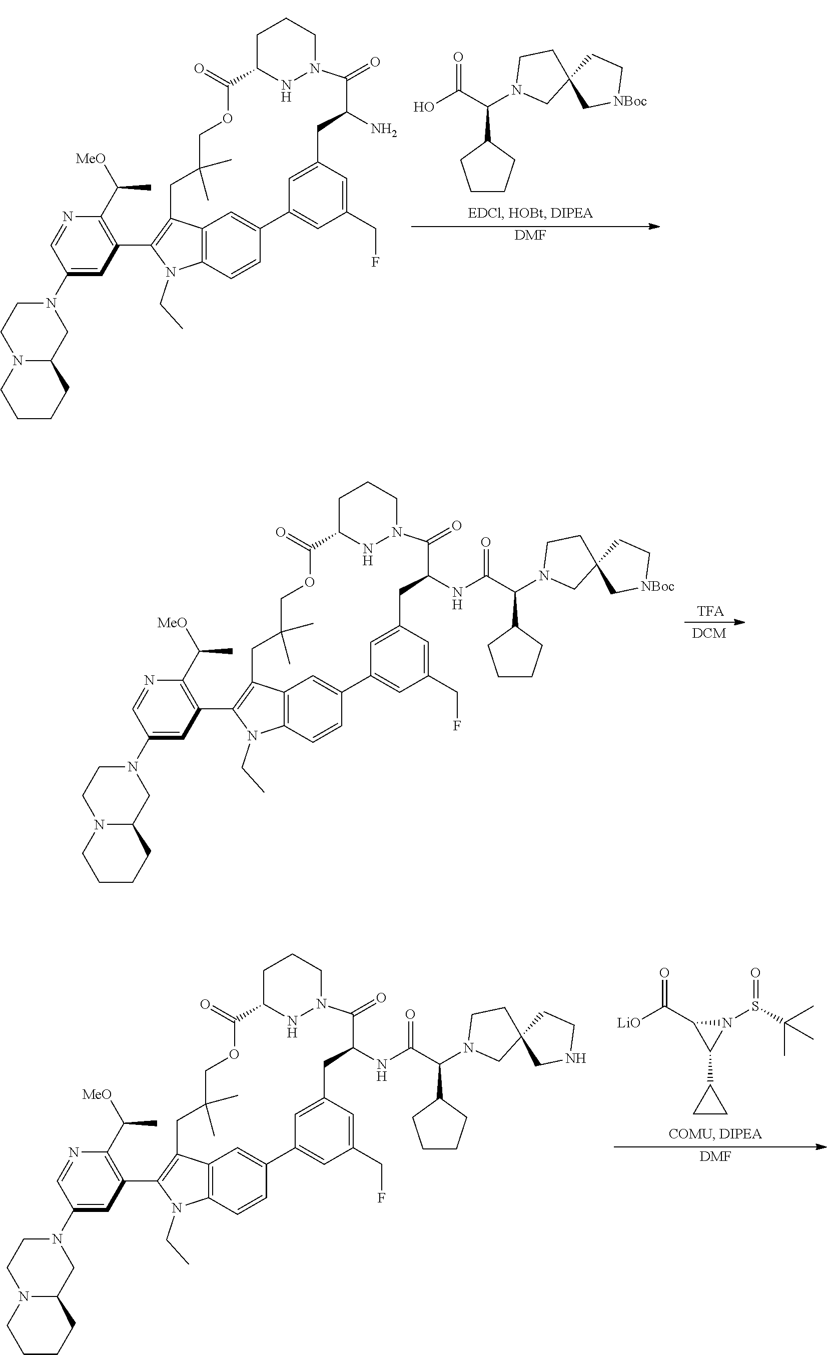

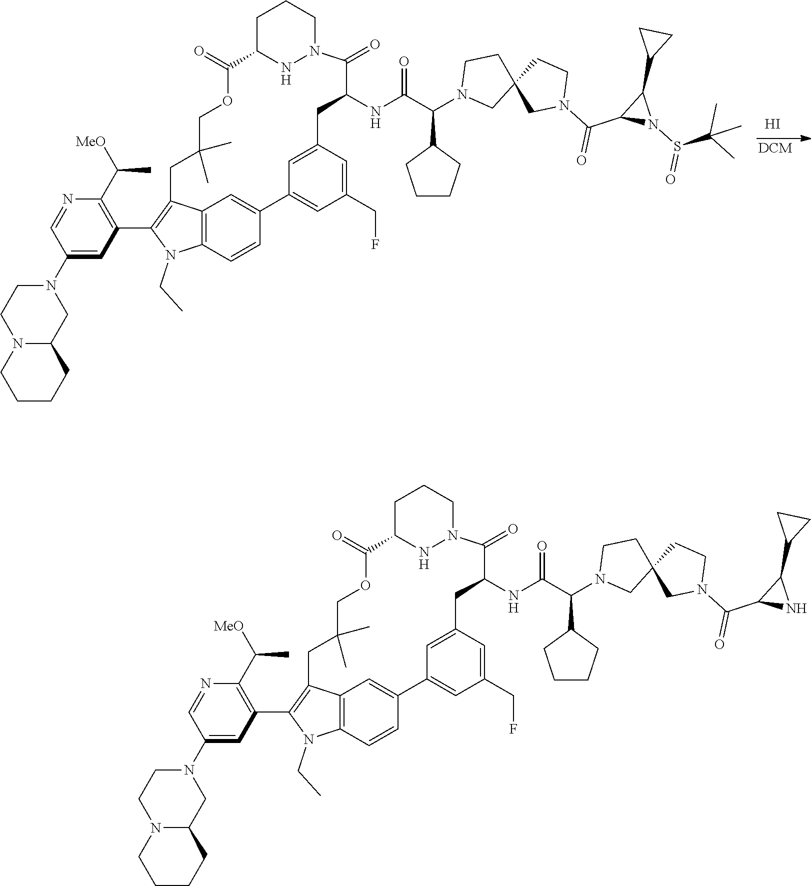

### Step 1: Synthesis of tert-butyl (5S)-7-((1 S)-1-cyclopentyl-2-(((6³S,4S)-1¹-ethyl-2⁵-(fluoromethyl)-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

To a solution of (6³S,4S)-4-amino-1¹-ethyl-1⁶-(fluoromethyl)-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (500 mg, 0.630 mmol), DIPEA (2.44 g, 18.90 mmol) and (S)-[(5S)-7-(tert-butoxycarbonyl)-2,7-diazaspiro[4.4]nonan-2-yl](cyclopentyl)acetic acid (266.34 mg, 0.756 mmol) in DCM (50 mL) was added HOBt (255.26 mg, 1.890 mmol) and EDCI (1.81 g, 9.450 mmol) at 0° C. The resulting mixture was stirred overnight at room temperature under a nitrogen atmosphere. The mixture was concentrated under reduced pressure. The residue was purified by reverse phase chromatography (0→100% MeCN/H₂O 0.1% NH₄HCO₃) to afford the product (523 mg, 83%) as a solid. LCMS (ESI) m/z [M+H] calcd for C₆₅H₉₀FN₉O₇: 1128.70; found: 1128.6

### Step 2: Synthesis of (2S)-2-cyclopentyl-N-((6³S,4S)-1¹-ethyl-2⁵-(fluoromethyl)-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

To a solution of tert-butyl (5S)-7-((1 S)-1-cyclopentyl-2-(((6³S,4S)-1¹-ethyl-2⁵-(fluoromethyl)-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (187 mg, 0.166 mmol) in DCM (2 mL) was added TFA (1 mL) at 0° C. The resulting mixture was stirred for 1 h at room temperature under a nitrogen atmosphere. The mixture was basified to pH 7˜8 with sat. aq. NaHCO₃. The resulting mixture was extracted with EtOAc (3×20 mL). The combined organic layers were washed with brine (3×20 mL), dried over anhydrous Na₂SO₄. After filtration, the filtrate was concentrated under reduced pressure. The crude product (154 mg, crude) as a solid was used in the next step directly without further purification.

LCMS (ESI) m/z [M+H] calcd for C₆₀H₈₂FN₉O₅: 1028.65; found: 1028.7

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-2⁵-(fluoromethyl)-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-cyclopentyl-N-((6³S,4S)-1¹-ethyl-2⁵-(fluoromethyl)-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (178 mg, 0.173 mmol), DIPEA (223.71 mg, 1.730 mmol) and lithium (2R,3R)-3-cyclopropyl-1-[(R)-2-methyl propane-2-sulfinyl]aziridine-2-carboxylate (123.19 mg, 0.519 mmol) in DMF (3 mL) was added COMU (88.90 mg, 0.208 mmol) at 0° C. The resulting mixture was stirred for 1 h at room temperature under a nitrogen atmosphere to afford (140 mg, 65%) as a solid. LCMS (ESI) m/z [M+Na]; calcd for C₇₀H₉₇FN₁₀O₇S: 1263.71; found: 1263.7

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-2⁵-(fluoromethyl)-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-2⁵-(fluoromethyl)-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (420 mg, 0.338 mmol) in THE (45 mL) was added HI (607 mg, 2.704 mmol) at 0° C. The resulting mixture was stirred for 30 min at 0° C. under a nitrogen atmosphere. The reaction was quenched by the addition of DIPEA (300 mg) within cooled EtOAc (50 mL) at 0° C. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by prep-TLC (10% MeOH/DCM) to afford the product (125 mg, 31%) as a solid.

LCMS (ESI) m/z [M+H] calcd for C₆₆H₈₉FN₁₀O₆: 1137.71; found: 1138.1

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-25-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

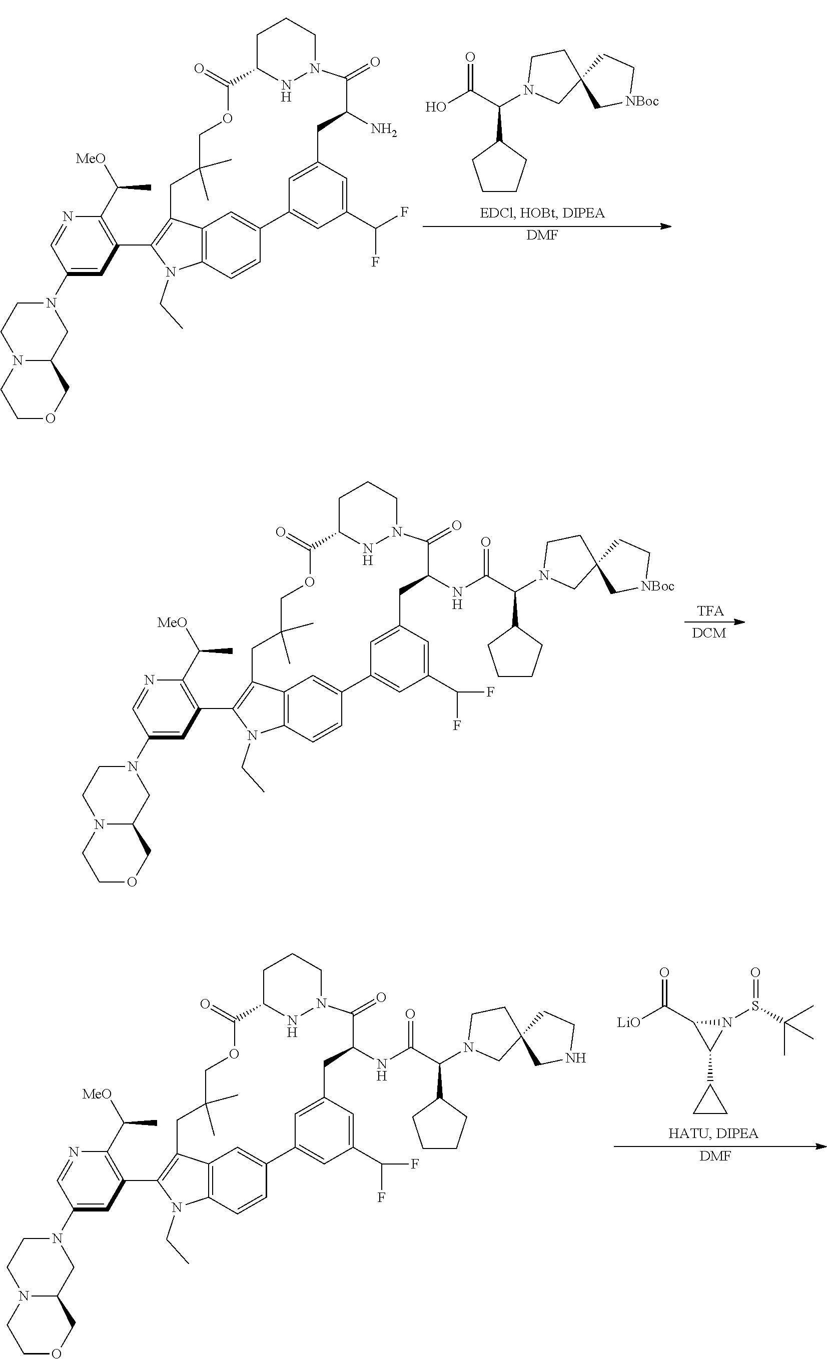

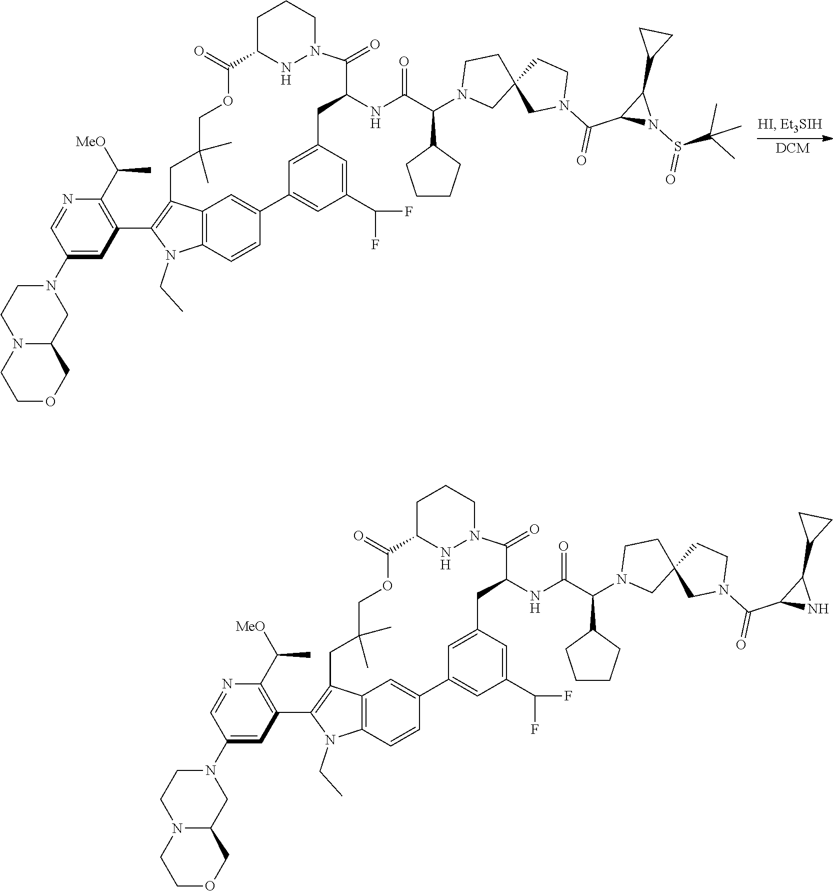

### Step 1: Synthesis of tert-butyl (5S)-7-((1S)-1-cyclopentyl-2-(((6³S,4S)-2⁵-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

Into a 50 mL round-bottom flask was added ((6³S,4S)-4-amino-2⁵-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (500 mg, 0.614 mmol), (S)-[(5S)-7-(tert-butoxycarbonyl)-2,7-diazaspiro[4.4]nonan-2-yl](cyclopentyl)acetic acid (649.53 mg, 1.842 mmol), DIPEA (2.38 g, 18.420 mmol), HOBt (415.00 mg, 3.070 mmol) and EDCI (3.30 g, 17.192 mmol) in DCM (5 mL). The resulting solution was stirred for 2 h at room temperature, then the resulting solution was diluted with DCM and washed with brine (2×50 mL). The mixture was dried over anhydrous Na₂SO₄ and concentrated under reduced pressure. The residue was purified by SFC chromatography (0→89% MeOH/CO₂) to afford the product (501 mg, 71%) as a solid. LCMS (ESI) m/z [M+H] calcd for C₆₄H₈₇F₂N₉O₈: 1148.67; found: 1148.4

### Step 2: Synthesis of (2S)-2-cyclopentyl-N-((6³S,4S)-25-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

Into a 50 mL round-bottom flask, was added tert-butyl (5S)-7-((1S)-1-cyclopentyl-2-(((6³S,4S)-2⁵-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)amino)-2-oxoethyl)-2,7-diazaspiro[4.4]nonane-2-carboxylate (700 mg, 0.610 mmol) in DCM (6 mL) and TFA (3 mL) at 0° C. The resulting solution was stirred for 1 h at 0° C. The mixture was concentrated under reduced pressure and adjusted to pH 8 by the addition of sat. aq. NaHCO₃ at 0° C. and extracted with 100 mL of (10% MeOH/DCM). The combined organic layers were washed with brine (3×50 mL), dried over anhydrous Na₂SO₄. After filtration, the filtrate was concentrated under reduced pressure. The crude product (613 mg, crude) as a solid was used in the next step directly without further purification. LCMS (ESI) m/z [M+H] calcd for C₅₉H₇₉F₂N₉O₆: 1048.62; found: 1048.6

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-2⁵-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

A mixture of (2S)-2-cyclopentyl-N-((6³S,4S)-25-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (590 mg, 0.563 mmol), lithium (2R,3R)-1-((R)-tert-butylsulfinyl)-3-cyclopropylaziridine-2-carboxylate (173.57 mg, 0.732 mmol), DIPEA (1.45 g, 11.260 mmol) and HATU (235.39 mg, 0.619 mmol) in DMF (6 mL) was stirred for 1 h at 0° C. The resulting mixture was purified by reverse phase chromatography (0→100% MeCN/H₂O 0.1% NH₄HCO₃) to afford the product (290 mg, 41% yield) as solid. LCMS (ESI) m/z [M+H] calcd C₆₉H₉₄F₂N₁₀O₈S: 1261.70; found: 1261.3

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-25-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-2⁵-(difluoromethyl)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (290 mg, 0.230 mmol) in THE (5 mL) was added Et₃SiH (267.28 mg, 2.30 mmol) and HI (235.22 mg, 1.84 mmol) dropwise at 0° C. The resulting mixture was stirred for 20 min at 0° C. under a nitrogen atmosphere. The reaction was added to EtOAc (100 mL) with DIPEA (600 mg). The resulting mixture was washed with brine (7×20 mL), dried over anhydrous Na₂SO₄. After filtration, the filtrate was concentrated under reduced pressure at room temperature. The residue was purified by prep-TLC (8% MeOH/DCM) to afford the product (71.7 mg, 24% yield) as a solid. LCMS (ESI) m/z [M+H] calcd for C₆₅H₈₆F₂N₁₀O₇: 1157.68; found: 1158.1

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-2⁵-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

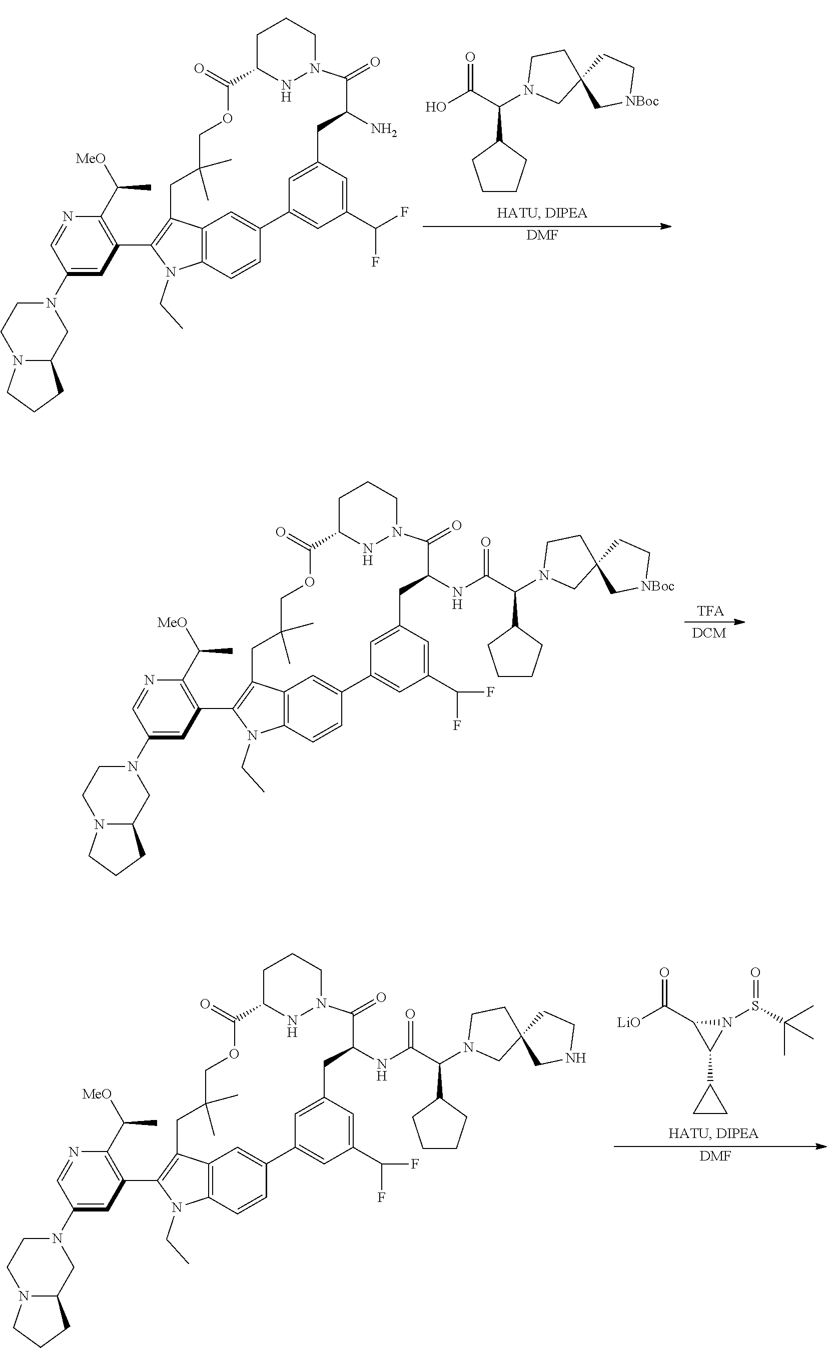

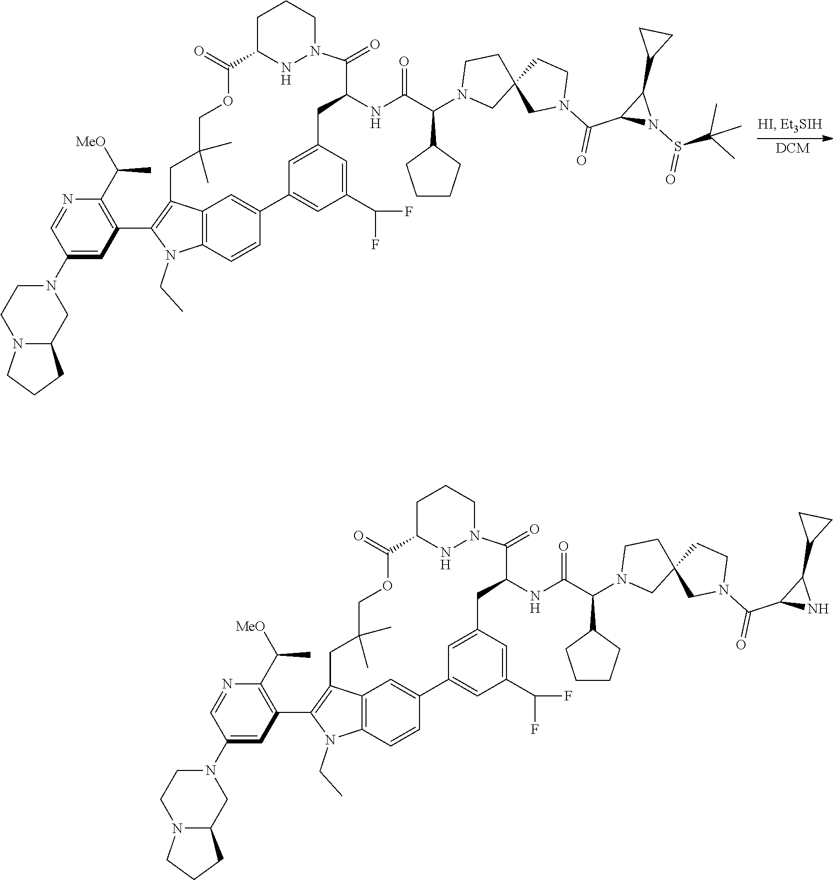

### Step 1: Synthesis of tert-butyl (5S)-7-((1 S)-1-cyclopentyl-2-(((6³S,4S)-25-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

Into a 50 mL round-bottom flask, was added (6³S,4S)-4-amino-25-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (560 mg, 0.702 mmol), (S)-2-((S)-7-(tert-butoxycarbonyl)-2,7-diazaspiro[4.4]nonan-2-yl)-2-cyclopentylacetic acid (296.82 mg, 0.842 mmol), DIPEA (1.36 g, 10.53 mmol), and HATU (293.51 mg, 0.772 mmol) in MeCN (6 mL). The resulting solution was stirred for 2 h at room temperature. The resulting solution was diluted with H₂O (10 mL) and extracted with EtOAc (100 mL) the organic layer was washed with brine (2×50 mL). The organic layer was dried over anhydrous Na₂SO₄ and concentrated under reduced pressure. The residue purified by silica gel column chromatography (18% MeOH/DCM) to afford the product (594 mg, 75% yield) as a solid. LCMS (ESI) m/z [M+H] calcd C₆₄H₈₇F₂N₉O₇: 1132.68; found: 1132.3

### Step 2: Synthesis of (2S)-2-cyclopentyl-N-((6³S,4S)-2⁵-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

Into a 50 mL round-bottom flask, was added tert-butyl (5S)-7-((1S)-1-cyclopentyl-2-(((6³S,4S)-25-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (580 mg, 0.512 mmol) in DCM (6 mL), and TFA (3 mL). The resulting solution was stirred for 2 h at room temperature. The mixture was quenched by the addition of sat. aq. NaHCO₃ (20 mL) at 0° C. and extracted with EtOAc (100 mL). The combined organic layers were washed with brine (3×40 mL), dried over anhydrous Na₂SO₄. After filtration, the filtrate was concentrated under reduced pressure. The crude product (509 mg, crude) as a solid was used in the next step directly without further purification.

LCMS (ESI) m/z [M+H] calcd for C₅₉H₇₉F₂N₉O₅: 1032.63; found: 1032.6

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-2⁵-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

A mixture of (2S)-2-cyclopentyl-N-((6³S,4S)-2⁵-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (499 mg, 0.483 mmol), lithium (2R,3R)-1-((R)-tert-butylsulfinyl)-3-cyclopropylaziridine-2-carboxylate(149.08 mg, 0.628 mmol), DIPEA (937.08 mg, 7.245 mmol) and HATU (202.17 mg, 0.531 mmol) in DMF (6 mL) was stirred for 1 h at 0° C. under an argon atmosphere. The resulting mixture was concentrated under reduced pressure then extracted with EtOAc (3×50 mL). The combined organic layers were washed with brine (3×30 mL), dried over anhydrous Na₂SO₄. After filtration, the filtrate was concentrated under reduced pressure. The crude product was purified by reverse phase chromatography (0→100% MeCN/H₂O 0.1% NH₄HCO₃) to afford the product (406 mg, 67% yield) as a solid. LCMS (ESI) m/z [M+H] calcd for C₆H₉₄F₂N₁₀O₇S: 1245.71; found: 1245.8

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-2⁵-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-2⁵-(difluoromethyl)-1¹-ethyl-1²-(5-((R)-hexahydropyrrolo[1,2-a]pyrazin-2(1H)-yl)-2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (406 mg, 0.326 mmol) in THE (5 mL) was added Et₃SiH (378.99 mg, 3.260 mmol) and HI (750.45 mg, 5.868 mmol) dropwise at 0° C. The resulting mixture was stirred for 20 min at 0° C. under a nitrogen atmosphere. The mixture basified to pH 7 to 8 with DIPEA (0.3 mL) at 0° C. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by prep-TLC (10% MeOH/DCM) to afford the product (140 mg, 38% yield) as a solid.

LCMS (ESI) m/z [M+H] calcd for C₆₅H₈₆F₂N₁₀O₆: 1141.68; found: 1142.1

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

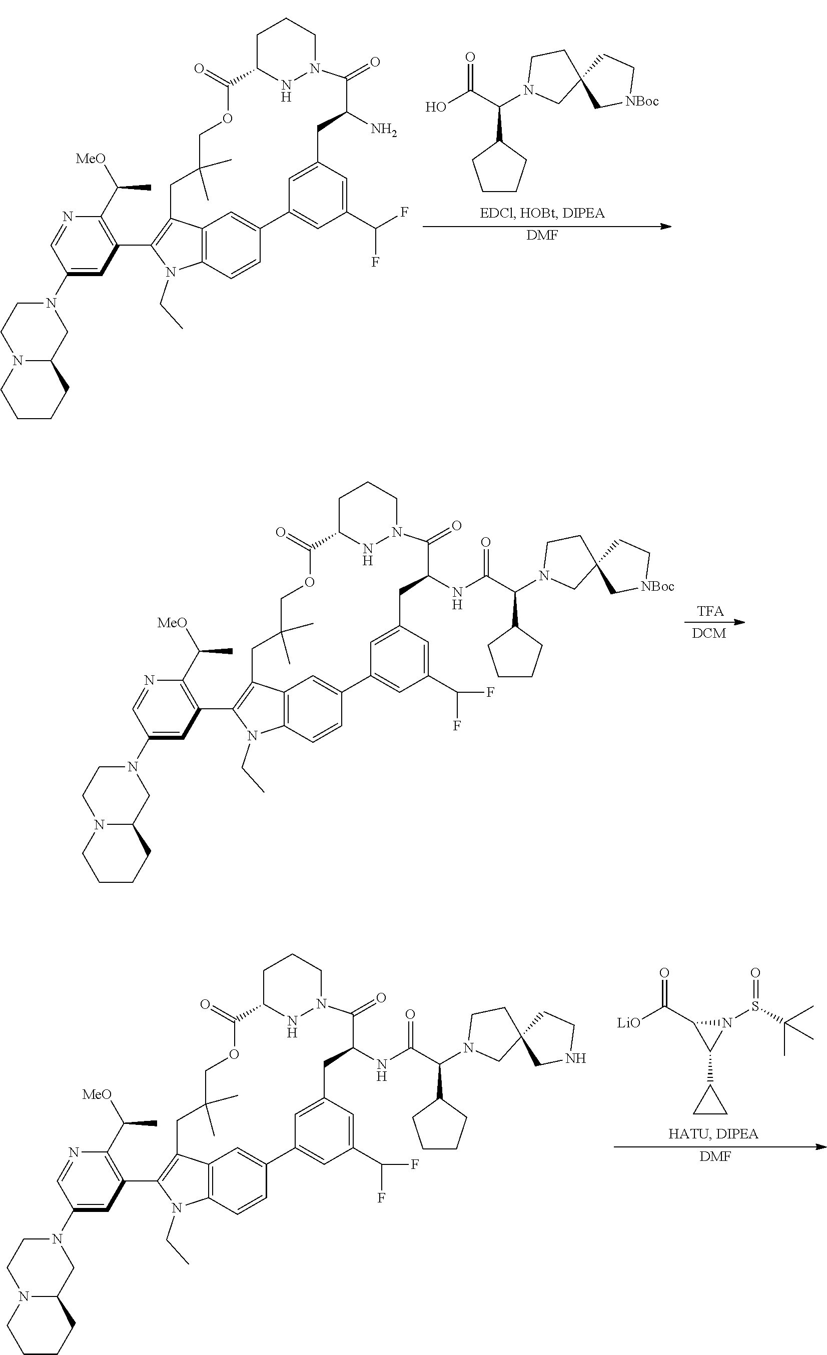

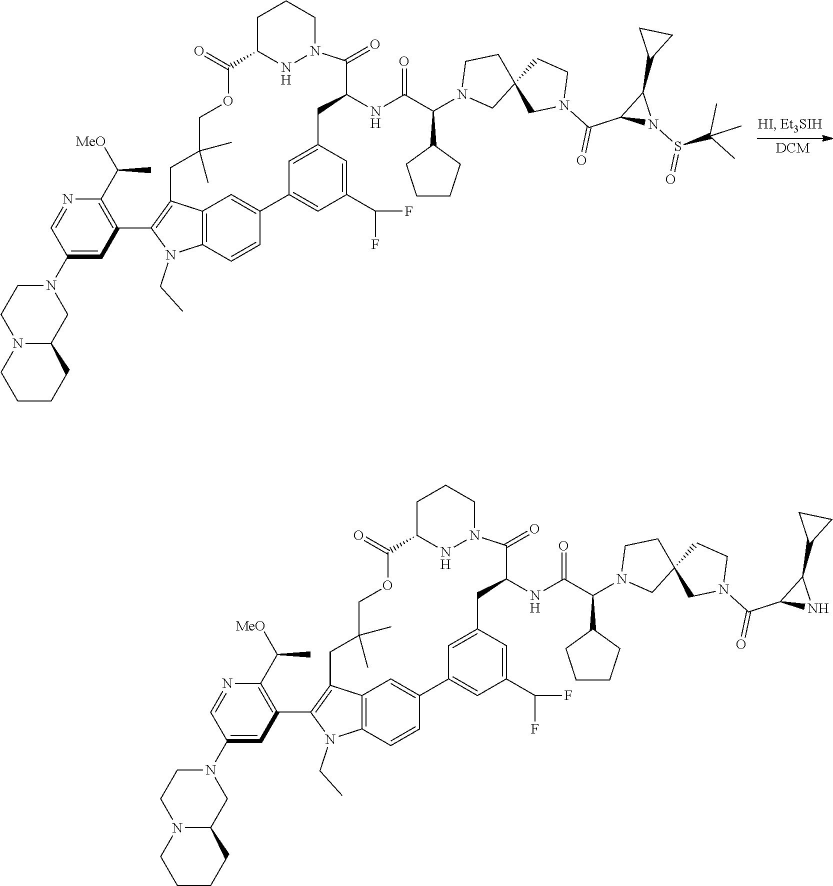

### Step 1: Synthesis of tert-butyl (5S)-7-((1 S)-1-cyclopentyl-2-(((6³S,4S)-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate

Into a 50 mL round-bottom flask, was added (6³S,4S)-4-amino-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (598 mg, 0.736 mmol), (S)-2-((S)-7-(tert-butoxycarbonyl)-2,7-diazaspiro[4.4]nonan-2-yl)-2-cyclopentylacetic acid (311.49 mg, 0.883 mmol), DIPEA (2.86 g, 22.080 mmol), HOBt (497.55 mg, 3.680 mmol) and EDCI (3.53 g, 18.400 mmol) in DCM (8 mL). The resulting solution was stirred for 2 h at room temperature. The resulting solution was diluted with H₂O (10 mL) and extracted with EtOAc (100 mL) then washed with brine (2×50 mL). The mixture was dried over anhydrous Na₂SO₄ and concentrated under reduced pressure. The residue purified by silica gel column chromatography (16% MeOH/DCM) to afford the product (568 mg, 67% yield) as a solid. LCMS (ESI) m/z [M+H] calcd for C₆₅H₈₉F₂N₉O₇: 1146.69; found: 1146.5

### Step 2: Synthesis of (2S)-2-cyclopentyl-N-((6³S,4S)-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide

Into a 50 mL round-bottom flask, was added tert-butyl (5S)-7-((1S)-1-cyclopentyl-2-(((6³S,4S)-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxoethyl)-2,7-diazaspiro[4.4]nonane-2-carboxylate (560 mg, 0.488 mmol) in DCM (6 mL), and TFA (3 mL). The resulting solution was stirred for 2 h at room temperature. The mixture was quenched by the addition of sat. aq. NaHCO₃ (20 mL) at 0° C. and extracted with EtOAc (100 mL). The combined organic layers were washed with brine (3×20 mL), dried over anhydrous Na₂SO₄. After filtration, the filtrate was concentrated under reduced pressure. The crude product (416 mg, 81% yield) as a solid was used in the next step directly without further purification.

LCMS (ESI) m/z [M+H] calcd for C₆₀H₈₁F₂N₉O₅: 1046.64; found: 1046.6

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((63S,4S)-25-(difluoromethyl)-11-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

A mixture of (2S)-2-cyclopentyl-N-((6³S,4S)-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-((S)-2,7-diazaspiro[4.4]nonan-2-yl)acetamide (400 mg, 0.382 mmol), lithium (2R,3R)-1-((R)-tert-butylsulfinyl)-3-cyclopropylaziridine-2-carboxylate (117.90 mg, 0.497 mmol), DIPEA (247.03 mg, 1.910 mmol) and HATU (159.89 mg, 0.420 mmol) in DMF (4 mL) was stirred for 1 h at 0° C. under an argon atmosphere. The resulting mixture was concentrated under reduced pressure. The resulting mixture was extracted with EtOAc (3×50 mL). The combined organic layers were washed with brine (3×30 mL), dried over anhydrous Na₂SO₄. After filtration, the filtrate was concentrated under reduced pressure. The crude product was purified by reverse phase chromatography (0→100% MeOH/H₂O 0.1% NH₄HCO₃) to afford the product (463 mg, 96% yield) as solid. LCMS(ESI) m/z [M+H] calcd for C₇₀H₉₆F₂N₁₀O₇S: 1259.72; found: 1259.5

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-2⁵-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide

To a solution of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((63S,4S)-25-(difluoromethyl)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)acetamide (360 mg, 0.286 mmol) in THF(4 mL) was added Et₃SiH (332.31 mg, 2.86 mmol) and HI (658.01 mg, 5.148 mmol) dropwise at 0° C. The resulting mixture was stirred for 20 min at 0° C. under a nitrogen atmosphere. The mixture basified to pH 7 to 8 with DIPEA (0.3 mL) and the resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by prep-TLC (10% MeOH/DCM) to afford the product (102 mg, 31% yield) as a solid. LCMS (ESI) m/z [M+H] calcd for C₆₆H₈₈F₂N₁₀O₆: 1155.70; found: 1156.1

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)acetamide

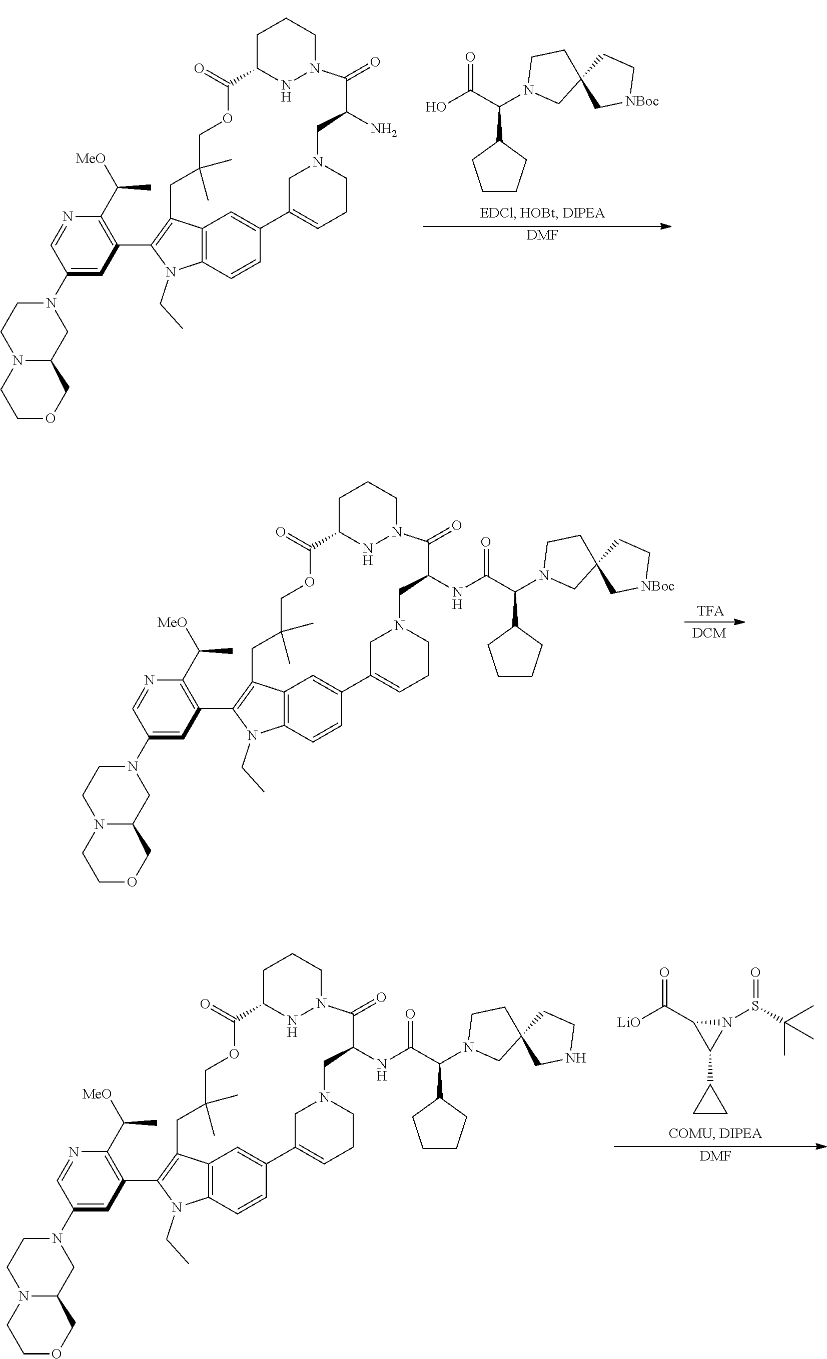

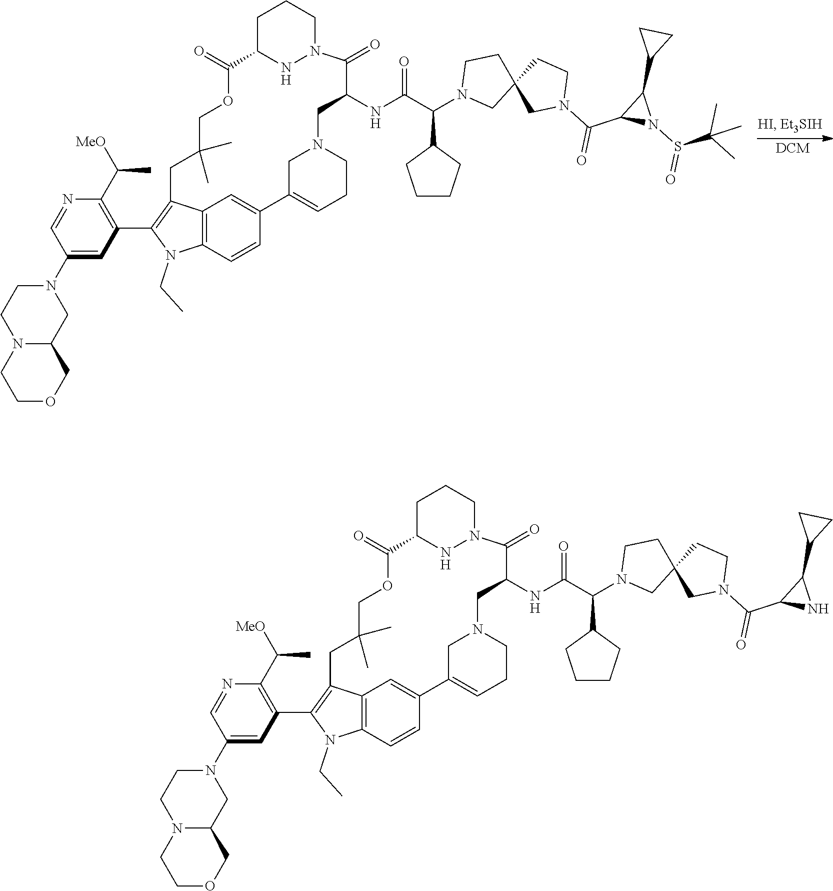

### Step 1: Synthesis of tert-butyl (5S)-7-((1 S)-1-cyclopentyl-2-(((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6(1,3)-pyridazina-2(5,1)- pyridinacycloundecaphane-4-yl)amino)-2-oxoethyl)-2,7-diazaspiro[4.4]nonane-2-carboxylate

To a stirred solution of (6³S,4S)-4-amino-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-5,7-dione (720 mg, 0.936 mmol) and (S)-2-((S)-7-(tert-butoxycarbonyl)-2,7-diazaspiro[4.4]nonan-2-yl)-2-cyclopentylacetic acid (495.02 mg, 1.404 mmol) in DCM (15 mL) was added EDC⋅HCl (1076.92 mg, 5.616 mmol) and HOBt (1265.13 mg, 9.360 mmol) dropwise at 0° C. under a nitrogen atmosphere. The resulting mixture was stirred for 2 h at 0° C. The mixture was basified to pH 8 with sat. aq. NaHCO₃. The resulting mixture was extracted with DCM (3×50 mL). The combined organic layers were washed with brine (3×50 mL), dried over anhydrous Na₂SO₄. After filtration, the filtrate was concentrated under reduced pressure. The residue was purified by silica gel column chromatography (2% MeOH/DCM) to afford the product (670 mg, 58% yield) as a solid. LCMS (ESI) m/z [M+H] calcd for C₆₂H₉₀N₁₀O₈: 1103.70; found: 1104.0

### Step 2: Synthesis of (2S)-2-cyclopentyl-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6(1,3)-pyridazina-2(5,1)- pyridinacycloundecaphane-4-yl)-2-((S)-2,7-diazaspiro[4.4]nonan-2-yl)acetamide

To a stirred solution of tert-butyl (5S)-7-((1S)-1-cyclopentyl-2-(((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)amino)-2-oxoethyl)-2,7-diazaspiro[4.4]nonane-2-carboxylate (670 mg, 0.607 mmol) in DCM (7 mL) was added TFA (7 mL) dropwise at 0° C. The resulting mixture was stirred for 1 h at 0° C. The mixture was basified to pH 8 with sat. aq. NaHCO₃. The resulting mixture was extracted with DCM (3×50 mL). The combined organic layers were washed with brine (3×50 mL), dried over anhydrous Na₂SO₄. After filtration, the filtrate was concentrated under reduced pressure. The crude product (640 mg, 84% yield) as a solid was used in the next step directly without further purification.

LCMS (ESI) m/z [M+H] calcd for C₅₇H₈₂N₁₀O₆: 1003.65; found: 1003.7

### Step 3: Synthesis of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6(1,3)-pyridazina-2(5,1)- pyridinacycloundecaphane-4-yl)acetamide

To a stirred solution of (2S)-2-cyclopentyl-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)-2-((S)-2,7-diazaspiro[4.4]nonan-2-yl)acetamide (640 mg, 0.638 mmol) and lithium (2R,3R)-1-((R)-tert-butylsulfinyl)-3-cyclopropylaziridine-2-carboxylate (378.32 mg, 1.595 mmol) in DMF (12 mL) was added DIPEA (824.40 mg, 6.380 mmol) and COMU (355.13 mg, 0.829 mmol) dropwise at 0° C. under a nitrogen atmosphere. The resulting mixture was stirred for 1 h at 0° C. The mixture was basified to pH 8 with sat. aq. NaHCO₃. The resulting mixture was extracted with DCM (3×50 mL). The combined organic layers were washed with brine (3×50 mL), dried over anhydrous Na₂SO₄.

After filtration, the filtrate was concentrated under reduced pressure. The residue was purified by prep-TLC (6% MeOH/DCM) to afford the product (490 mg, 57% yield) as a solid. LCMS (ESI) m/z [M+H] calcd for C₆₇H₉₇N₁₁O₈S: 1216.73; found: 1217.1

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-2¹ 2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)acetamide

To a stirred solution of (2S)-2-((5S)-7-((2R,3R)-1-(tert-butylsulfinyl)-3-cyclopropylaziridine-2-carbonyl)-2,7-diazaspiro[4.4]nonan-2-yl)-2-cyclopentyl-N-((6³S,4S)-1¹-ethyl-1²-(5-((S)-hexahydropyrazino[2,1-c][1,4]oxazin-8(1H)-yl)-2-((S)-1-methoxyethyl)pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6(1,3)-pyridazina-2(5,1)- pyridinacycloundecaphane-4-yl)acetamide (490 mg, 0.403 mmol) and Et₃SiH (702.45 mg, 6.045 mmol) in THE (12 mL) was added HI (515.17 mg, 4.030 mmol) dropwise at 0° C. under a nitrogen atmosphere. The resulting mixture was stirred for 30 min at 0° C. The mixture was basified to pH 8 with DIPEA. The resulting mixture was extracted with EtOAc (3×15 mL), dried over anhydrous Na₂SO₄. After filtration, the filtrate was concentrated under reduced pressure. The residue was purified by prep-TLC (8% MeOH/DCM) to afford the product (150 mg, 32% yield) as an solid. LCMS (ESI) m/z [M+H] calcd for C₆₃H₈₉N₁₁O₇: 1112.70; found: 1113.0

### Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)acetamide

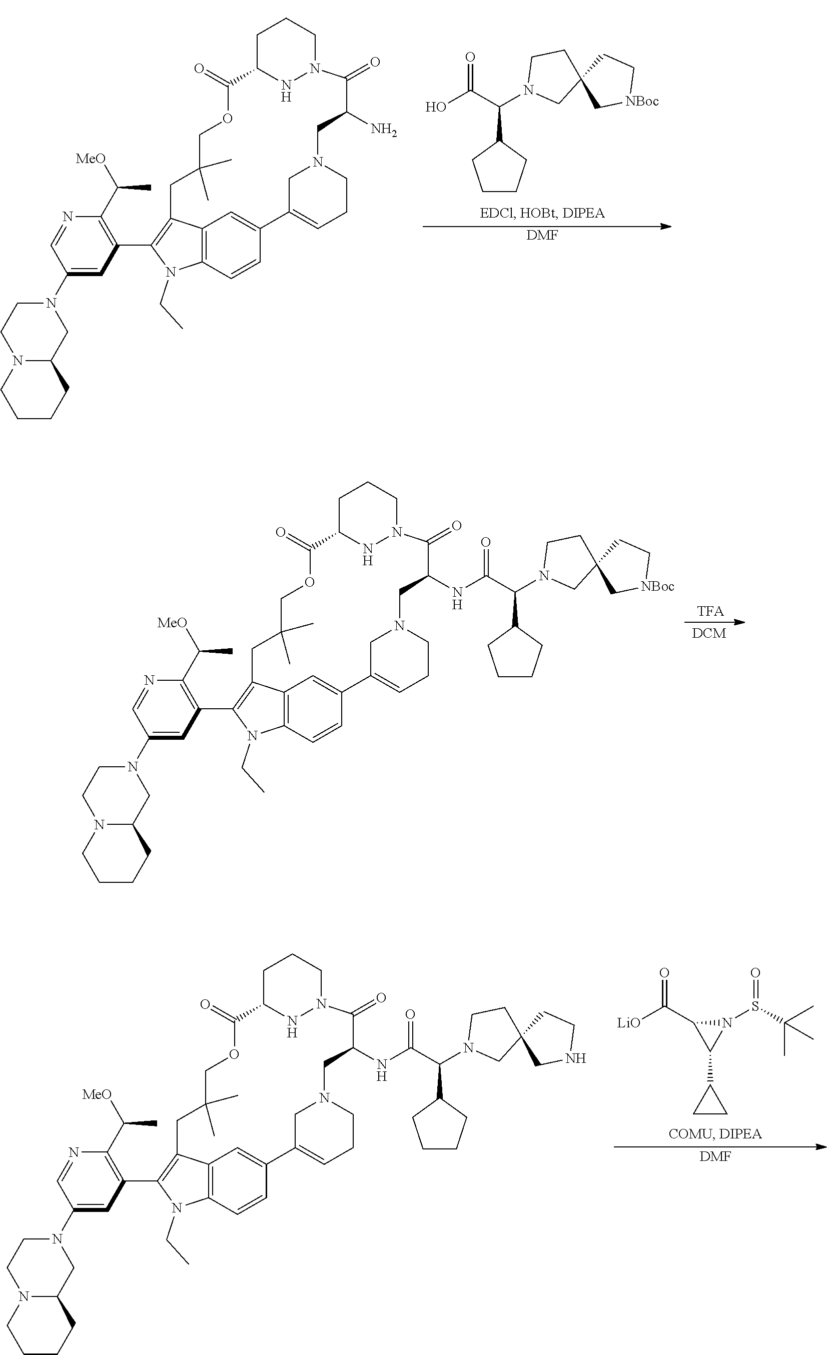

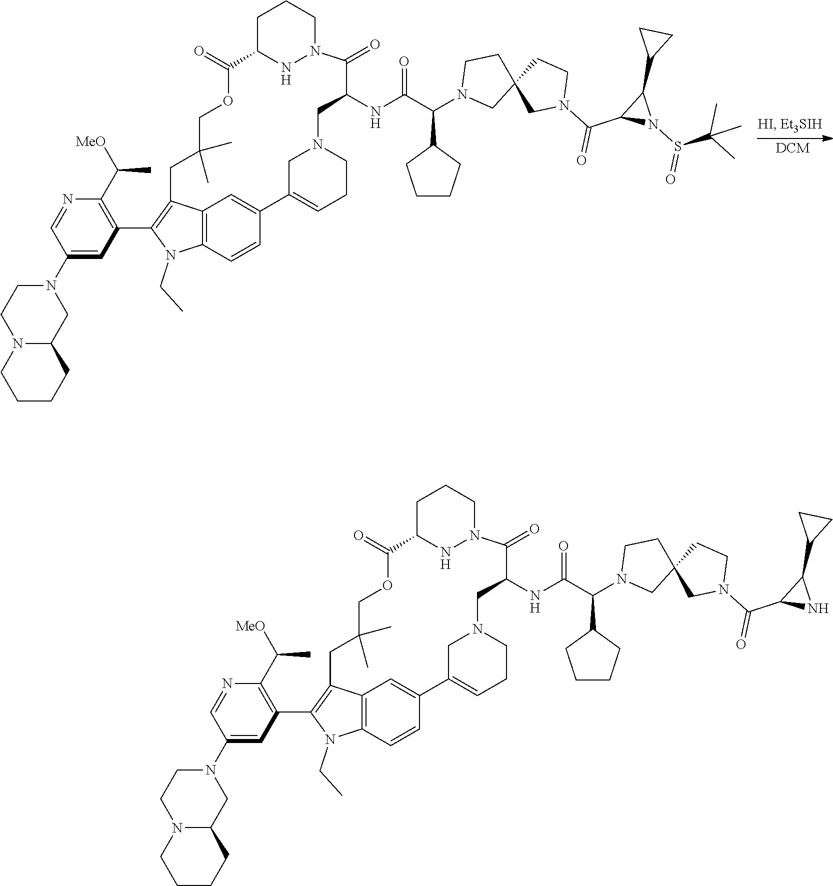

### Step 1: Synthesis of tert-butyl (5S)-7-((1S)-1-cyclopentyl-2-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)amino)-2-oxoethyl)-2,7-diazaspiro[4.4]nonane-2-carboxylate

To a stirred solution of (6³S,4S)-4-amino-1¹-ethyl-1²-(2-((S)-1-methoxyethyl)-5-((R)-octahydro-2H-pyrido[1,2-a]pyrazin-2-yl)pyridin-3-yl)-10,10-dimethyl-2¹ 2²,2³,2⁶,6¹⁷,6²,6³,6⁴,6⁵,6⁶-decahydro-1¹H-8-oxa-1(5,3)-indola-6(1,3)-pyridazina-2(5,1)-pyridinacycloundecaphane-5,7-dione (260 mg, 0.339 mmol) and (S)-2-((S)-7-(tert-butoxycarbonyl)-2,7-diazaspiro[4.4]nonan-2-yl)-2-cyclopentylacetic acid (179 mg, 0.509 mmol) in DCM (6 mL) was added DIPEA (876 mg, 6.78 mmol) and EDC⋅HCl (390 mg, 2.034 mmol) and HOBt (458 mg, 3.390 mmol) dropwise at 0° C. under a nitrogen atmosphere for 2 h. The mixture was basified to pH 8 with sat. aq. NaHCO₃. The resulting mixture was extracted with DCM (3×50 mL). The combined organic layers were washed with brine (3×50 mL), dried over anhydrous Na₂SO₄. After filtration, the filtrate was concentrated under reduced pressure. The residue was purified by silica gel column chromatography (2% MeOH/DCM) to afford the product (280 mg, 75% yield) as a solid. LCMS (ESI) m/z [M+H] calcd for C₆₃H₉₂N₁₀O₇: 1101.72; found: 1102.1

### Step 2: Synthesis of (2S)-2-cyclopentyl-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6(1,3)-pyridazina-2(5,1)-pyridinacycloundecaphane-4-yl)-2-((S)-2,7-diazaspiro[4.4]nonan-2-yl)acetamide

To a stirred solution of tert-butyl (5S)-7-((1S)-1-cyclopentyl-2-(((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)amino)-2-oxoethyl)-2,7-diazaspiro[4.4]nonane-2-carboxylate (280 mg, 0.254 mmol) in DCM (3 mL) was added TFA (3 mL) dropwise at 0° C. under a nitrogen atmosphere for 1 h. The mixture was basified to pH 8 with sat. aq. NaHCO₃. The resulting mixture was extracted with DCM (3×50 mL). The combined organic layers were washed with brine (3×50 mL), dried over anhydrous Na₂SO₄. After filtration, the filtrate was concentrated under reduced pressure. The crude product (240 mg, 94% yield) as a solid was used in the next step directly without further purification. LCMS (ESI) m/z [M+H] calcd for C₅₈H₈₄N₁₀O₅: 1001.67; found: 1001.6

### Step 3: Synthesis of (2S)-2-cyclopentyl-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)acetamide

To a stirred solution of (2S)-2-cyclopentyl-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)-pyridinacycloundecaphane-4-yl)-2-((S)-2,7-diazaspiro[4.4]nonan-2-yl)acetamide (150 mg, 0.15 mmol) and lithium (2R,3R)-3-cyclopropyl-1-((R)-p-tolylsulfinyl)aziridine-2-carboxylate (102 mg, 0.375 mmol) in DMF (2 mL) was added COMU (83 mg, 0.195 mmol) and DIPEA (194 mg, 1.50 mmol) dropwise at 0° C. under a nitrogen atmosphere and stirred for 1 h. The mixture was basified to pH 8 with sat. aq. NaHCO₃. The resulting mixture was extracted with DCM (3×50 mL). The combined organic layers were washed with brine (3×50 mL), dried over anhydrous Na₂SO₄. After filtration, the filtrate was concentrated under reduced pressure. The residue was purified by prep-TLC (DCM/MeOH 6%) to afford the product (140 mg, 75% yield) as a solid. LCMS (ESI) m/z [M+H]/2; calcd for C₇₁H₉₇N₁₁O₇S: 1248.74; found: 625.2

### Step 4: Synthesis of (2S)-2-cyclopentyl-2-((S)-7-((2R,3R)-3-cyclopropylaziridine-2-carbonyl)-2,7-diazaspiro[4.4]nonan-2-yl)-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1 (5,3)-indola-6(1,3)-pyridazina-2(5,1)-pyridinacycloundecaphane-4-yl)acetamide

To a stirred solution of (2S)-2-cyclopentyl-2-((S)-7-((2R,3R)-3-cyclopropyl-1-((R)-p-tolylsulfinyl)aziridine-2-carbonyl)-2,7-diazaspiro[4.4]nonan-2-yl)-N-((6³S,4S)-1¹-ethyl-1²-(2-((S)-1-methoxyethyl)-5-((R)-octahydro-2H-pyrido[1,2-a]pyrazin-2-yl)pyridin-3-yl)-10,10-dimethyl-5,7-dioxo-2¹,2²,2³,2⁶,6¹,6²,6³,6⁴,6⁵,6⁶-decahydro-1¹H-8-oxa-1(5,3)-indola-6(1,3)-pyridazina-2(5,1)- pyridinacycloundecaphane-4-yl)acetamide (90 mg, 0.072 mmol) in THE (1 mL) was added MeMgBr (1 M, 0.576 mL) at −78° C. under N₂. Then the mixture was stirred at −78° C. for 1 h. The reaction mixture was quenched with sat. aq. NH₄Cl (20 mL) at 0° C. Then the aqueous phase was extracted with EtOAc (3×15 mL). The combined organic layers were washed with brine (30 mL), dried with anhydrous Na₂SO₄, filtered, and concentrated under reduced pressure to give the crude product. The residue was purified by prep-TLC (8% MeOH/DCM) to afford the product (55 mg, 66% yield) as a solid. LCMS (ESI) m/z [M+H] calcd for C₆₄H₉₁N₁₁O₆: 1110.73; found: 1111.1

TABLE F6
Exemplary Compounds Prepared by
Methods of the Present Invention

| | Molecular | | Observed MW |
|---|---|---|---|
| Ex# | Formula | Calculated MW | LCMS (ESI) m/z |
| FA1 | C₅₁H₆₆N₈O₇S | [M + H] = 935.49 | [M + H] = 935.4 |
| FA2 | C₅₅H₇₃N₉O₆S | [M + H] = 988.55 | [M + H] = 988.5 |
| FA3 | C₅₇H₇₅N₉O₆S | [M + H] = 1014.57 | [M + H] = 1014.5 |
| FA4 | C₅₂H₆₉N₉O₆S | [M + H] = 948.52 | [M + H] = 948.6 |
| FA5 | C₅₂H₆₉N₉O₆S | [M + H] = 948.52 | [M + H] = 948.3 |
| FA6 | C₅₂H₆₈N₈O₇S | [M + H] = 949.50 | [M + H] = 949.5 |
| FA7 | C₅₄H₇₀N₈O₇S | [M + H] = 975.52 | [M + H] = 975.5 |
| FA8 | C₅₄H₇₁N₉O₆S | [M + H] = 974.53 | [M + H] = 974.5 |
| FA9 | C₅₅H₇₃N₉O₆S | [M + H] = 988.55 | [M + H] = 988.5 |
| FA10 | C₅₄H₇₀N₈O₆ | [M + H] = 927.55 | [M + H] = 927.6 |
| FA11 | C₅₈H₇₄N₈O₇ | [M + H] = 995.58 | [M + H] = 995.5 |
| FA12 | C₅₄H₆₉N₇O₇ | [M + H] = 928.54 | [M + H] = 928.5 |
| FA13 | C₅₇H₇₄N₈O₆ | [M + H] = 967.58 | [M + H] = 967.5 |
| FA14 | C₅₅H₇₂N₈O₆ | [M + H] = 941.57 | [M + H] = 941.6 |
| FA15 | C₅₃H₇₂N₈O₇ | [M + H] = 933.56 | [M + H] = 933.6 |
| FA16 | C₅₆H₇₇N₉O₆ | [M + H] = 972.61 | [M + H] = 972.5 |
| FA17 | C₅₆H₇₂F₂N₈O₆ | [M + H] = 991.56 | [M + H] = 992.2 |
| FA18 | C₅₈H₇₄F₂N₈O₆ | [M + H] = 1017.58 | [M + H] = 1017.4 |
| FA19 | C₅₅H₇₇N₉O₇ | [M + H] = 976.61 | [M + H] = 976.6 |
| FA20 | C₅₃H₇₅N₉O₇ | [M + H] = 950.59 | [M + H] = 950.6 |
| FA21 | C₅₈H₇₅FN₈O₆ | [M + H] = 999.59 | [M + H] = 999.5 |
| FA22 | C₅₆H₇₃FN₈O₆ | [M + H] = 973.57 | [M + H] = 973.5 |
| FA23 | C₆₁H₈₃N₁₁O₇S | [M + H] = 1114.63 | [M + H] = 1013.0 |
| FA24 | C₅₈H₇₈N₁₀O₈S | [M + H] = 1075.58 | [M + H] = 1076.1 |
| FA25 | C₆₂H₈₅N₁₁O₆S | [M + H] = 1112.65 | [M + H] = 1113.0 |
| FA26 | C₆₄H₈₃F₃N₁₀O₇ | [M + H] = 1161.65 | [M + H] = 1162.1 |
| FA27 | C₆₅H₈₅F₃N₁₀O₆ | [M + H] = 1159.67 | [M + H] = 1160.0 |
| FA28 | C₆₄H₈₆N₁₀O₇ | [M + H] = 1107.68 | [M + H] = 1107.9 |
| FA29 | C₆₂H₈₄N₁₀O₇ | [M + H] = 1081.66 | [M + H] = 1082.0 |
| FA30 | C₆₅H₈₈N₁₀O₆ | [M + H] = 1105.70 | [M + H] = 1106.1 |
| FA31 | C₆₄H₈₆N₁₀O₆ | [M + H] = 1091.68 | [M + H] = 1092.1 |
| FA32 | C₆₃H₉₁N₁₁O₇ | [M + H] = 1114.72 | [M + H] = 1115.0 |
| FA33 | C₆₀H₈₄F₃N₁₁O₇ | [M + H] = 1128.66 | [M + H] = 1129.0 |
| FA34 | C₆₅H₈₇FN₁₀O₇ | [M + H] = 1139.68 | [M + H] = 1140.1 |
| FA35 | C₆₅H₈₇FN₁₀O₆ | [M + H] = 1123.69 | [M + H] = 1124.1 |
| FA36 | C₆₆H₈₉FN₁₀O₆ | [M + H] = 1137.71 | [M + H] = 1138.1 |
| FA37 | C₆₅H₈₆F₂N₁₀O₇ | [M + H] = 1157.68 | [M + H] = 1158.1 |
| FA38 | C₆₅H₈₆F₂N₁₀O₆ | [M + H] = 1141.68 | [M + H] = 1142.1 |
| FA39 | C₆₆H₈₈F₂N₁₀O₆ | [M + H] = 1155.70 | [M + H] = 1156.1 |
| FA40 | C₆₃H₈₉N₁₁O₇ | [M + H] = 1112.70 | [M + H] = 1113.0 |
| FA41 | C₆₄H₉₁N₁₁O₆ | [M + H] = 1110.73 | [M + H] = 1111.1 |
| FA42 | C₅₆H₇₅N₉O₆S | [M + H] = 1002.57 | [M + H] = 1002.9 |
| FA43 | C₅₄H₇₁N₉O₆S | [M + H] = 974.54 | [M + H] = 974.6 |
| FA44 | C₅₁H₆₇N₉O₆S | [M + H] = 934.50 | [M + H] = 934.5 |
| FB1 | C₆₀H₈₇N₁₁O₇ | [M + H] = 1074.69 | [M + H] = 1074.8 |
| FB2 | C₆₄H₉₃N₁₁O₈ | [M + H] = 1144.73 | [M + H] = 1144.9 |
| FB3 | C₆₀H₈₇N₁₁O₈ | [M + H] = 1090.68 | [M + H] = 1091.0 |
| FB4 | C₆₂H₈₁F₃N₁₀O₇ | [M + H] = 1135.63 | [M + H] = 1135.6 |
| FB5 | C₆₁H₈₀F₃N₁₁O₇S | [M + H] = 1168.60 | [M + H] = 1168.8 |
| FB6 | C₅₄H₆₈F₃N₉O₆S | [M + H] = 1028.51 | [M + H] = 1028.5 |
| FB7 | C₅₈H₇₉N₁₁O₇S | [M + H] = 1074.60 | [M + H] = 1075.0 |
| FB8 | C₆₄H₈₆N₁₀O₇ | [M + H] = 1107.68 | [M + H] = 1108.1 |
| FB9 | C₆₁H₈₃N₁₁O₇S | [M + H] = 1114.63 | [M + H] = 1114.9 |
| FB10 | C₆₂H₈₉N₁₁O₇ | [M + H] = 1100.71 | [M + H] = 1100.5 |
| FB11 | C₆₀H₈₄F₃N₁₁O₈ | [M + H] = 1144.66 | [M + H] = 1145.0 |
| FB12 | C₆₂H₈₆F₃N₁₁O₈ | [M + H] = 1170.67 | [M + H] = 1171.0 |
| FB13 | C₅₉H₈₁N₁₁O₇S | [M + H] = 1088.61 | [M + H] = 1088.9 |
| FB14 | C₆₃H₈₉N₁₁O₆ | [M + H] = 1096.71 | [M + H] = 1097.1 |
| FB15 | C₆₂H₈₉N₁₁O₈ | [M + H] = 1116.70 | [M + H] = 1117.0 |
| FB16 | C₅₂H₆₉N₉O₆S | [M + H] = 948.52 | [M + H] = 948.5 |
| FB17 | C₅₈H₇₈N₁₀O₇S | [M + H] = 1059.59 | [M + H] = 1059.6 |
| FB18 | C₅₈H₇₉N₉O₇S | [M + H] = 1046.59 | [M + H] = 1046.5 |
| FB19 | C₆₁H₈₃N₁₁O₆S | [M + H] = 1098.64 | [M + H] = 1099.0 |
| FB20 | C₅₃H₆₆F₃N₉O₇S | [M + H] = 1030.49 | [M + H] = 1030.4 |
| FB21 | C₅₁H₆₄F₃N₉O₇S | [M + H] = 1004.47 | [M + H] = 1004.5 |
| FB22 | C₅₀H₆₂F₃N₉O₇S | [M + H] = 990.46 | [M + H] = 990.3 |
| FB26 | C₅₄H₆₈F₃N₉O₆S | [M + H] = 1028.51 | [M + H] = 1028.5 |
| FB27 | C₅₂H₆₆F₃N₉O₆S | [M + H] = 1002.49 | [M + H] = 1002.4 |
| FB28 | C₅₁H₆₄F₃N₉O₆S | [M + H] = 988.48 | [M + H] = 988.5 |
| FB29 | C₅₆H₇₀F₃N₉O₇S | [M + H] = 1070.52 | [M + H] = 1070.5 |
| FB30 | C₅₄H₆₈F₃N₉O₇S | [M + H] = 1044.50 | [M + H] = 1044.5 |
| FB31 | C₅₇H₇₂F₃N₉O₆S | [M + H] = 1068.54 | [M + H] = 1068.5 |
| FB32 | C₅₅H₇₀F₃N₉O₆S | [M + H] = 1042.52 | [M + H] = 1042.4 |
| FB33 | C₅₄H₆₈F₃N₉O₆S | [M + H] = 1028.51 | [M + H] = 1028.5 |
| FB34 | C₅₃H₆₆F₃N₉O₇S | [M + H] = 1030.49 | [M + H] = 1030.4 |
| FB37 | C₆₁H₈₃N₁₁O₆S | [M + H] = 1098.64 | [M + H] = 1098.8 |
| FB38 | C₆₀H₈₁N₁₁O₇S | [M + H] = 1100.61 | [M + H] = 1100.9 |
| FB39 | C₆₁H₈₃N₁₁O₇ | [M + H] = 1082.66 | [M + H] = 1082.6 |
| FB40 | C₅₄H₆₈F₃N₉O₇ | [M + H] = 1012.53 | [M + H] = 1012.7 |
| FB41 | C₅₅H₇₄F₃N₉O₆ | [M + H] = 1014.58 | [M + H] = 1014.8 |
| FB42 | C₆₄H₈₅FN₁₀O₆ | [M + H] = 1109.67 | [M + H] = 1109.8 |
| FB44 | C₅₃H₆₆F₃N₉O₈ | [M + H] = 1014.51 | [M + H] = 1014.6 |
| FB45 | C₅₄H₇₂F₃N₉O₇ | [M + H] = 1016.56 | [M + H] = 1016.7 |
| FB46 | C₆₃H₈₃FN₁₀O₇ | [M + H] = 1111.65 | [M + H] = 1111.7 |
| FB47 | C₆₂H₈₅N₁₁O₇S | [M + H] = 1128.65 | [M + H] = 1129.0 |
| FB48 | C₆₀H₈₃N₁₁O₇S | [M + H] = 1102.63 | [M + H] = 1102.7 |
| FB49 | C₆₂H₈₂F₃N₁₁O₇S | [M + H] = 1182.62 | [M + H] = 1183.0 |
| FB50 | C₆₀H₈₀F₃N₁₁O₇S | [M + H] = 1156.60 | [M + H] = 1156.7 |
| FB52 | C₆₀H₈₂F₃N₁₁O₇ | [M + H] = 1126.65 | [M + H] = 1126.8 |
| FB55 | C₆₁H₇₉F₃N₁₀O₇ | [M + H] = 1121.62 | [M + H] = 1121.7 |
| FB56 | C₅₈H₇₆F₃N₁₁O₇S | [M + H] = 1128.57 | [M + H] = 1128.8 |
| FB58 | C₆₁H₈₄F₃N₁₁O₆ | [M + H] = 1124.67 | [M + H] = 1125.0 |
| FB59 | C₆₀H₈₄F₃N₁₁O₇ | [M + H] = 1128.66 | [M + H] = 1128.7 |
| FB60 | C₆₂H₈₁F₃N₁₀O₆ | [M + H] = 1119.64 | [M + H] = 1119.8 |
| FB61 | C₅₉H₇₈F₃N₁₁O₆S | [M + H] = 1126.59 | [M + H] = 1126.7 |
| FB62 | C₆₃H₈₆F₃N₁₁O₆ | [M + H] = 1150.68 | [M + H] = 1150.7 |
| FB63 | C₆₁H₈₄F₃N₁₁O₇ | [M + H] = 1140.66 | [M + H] = 1140.8 |
| FB64 | C₆₁H₈₇N₁₁O₇ | [M + H] = 1086.69 | [M + H] = 1087.2 |
| FB65 | C₆₃H₈₆F₃N₁₁O₇ | [M + H] = 1166.68 | [M + H] = 1166.7 |
| FB67 | C₅₉H₈₂F₃N₁₁O₉ | [M + H] = 1146.64 | [M + H] = 1146.9 |
| FB68 | C₆₁H₇₉F₃N₁₀O₈ | [M + H] = 1137.61 | [M + H] = 1137.8 |
| FB69 | C₅₈H₇₆F₃N₁₁O₈S | [M + H] = 1144.57 | [M + H] = 1144.8 |
| FB71 | C₆₄H₈₄F₃N₁₁O₇S | [M + H] = 1208.63 | [M + H] = 1208.9 |
| FB72 | C₆₂H₈₂F₃N₁₁O₇S | [M + H] = 1182.62 | [M + H] = 1182.8 |
| FB73 | C₆₂H₈₅N₁₁O₇S | [M + H] = 1128.65 | [M + H] = 1128.7 |
| FB74 | C₆₄H₈₇N₁₁O₇S | [M + H] = 1154.66 | [M + H] = 1154.9 |
| FB75 | C₅₉H₈₂F₃N₁₁O₈ | [M + H] = 1130.64 | [M + H] = 1130.9 |
| FB76 | C₆₁H₇₉F₃N₁₀O₇ | [M + H] = 1121.62 | [M + H] = 1121.9 |
| FB77 | C₅₈H₇₆F₃N₁₁O₇S | [M + H] = 1128.57 | [M + H] = 1128.8 |
| FB84 | C₆₂H₈₆F₃N₁₁O₇ | [M + H] = 1154.68 | [M + H] = 1154.8 |
| FB85 | C₆₄H₈₃F₃N₁₀O₆ | [M + H] = 1145.66 | [M + H] = 1145.9 |
| FB86 | C₆₁H₈₀F₃N₁₁O₆S | [M + H] = 1152.61 | [M + H] = 1152.8 |
| FB87 | C₅₉H₈₅N₁₁O₈ | [M + H] = 1076.67 | [M + H] = 1077.1 |
| FB88 | C₆₁H₈₂N₁₀O₇ | [M + H] = 1067.65 | [M + H] = 1067.9 |
| FB89 | C₅₉H₇₈F₃N₁₁O₇S | [M + H] = 1142.59 | [M + H] = 1142.8 |
| FB90 | C₆₂H₈₉N₁₁O₇ | [M + H] = 1100.70 | [M + H] = 1101.1 |
| FB91 | C₆₀H₇₈F₃N₁₁O₇S | [M + H] = 1154.59 | [M + H] = 1154.8 |
| FB92 | C₆₃H₈₁F₃N₁₀O₇ | [M + H] = 1147.63 | [M + H] = 1147.9 |
| FB94 | C₆₀H₇₈F₃N₁₁O₈S | [M + H] = 1170.58 | [M + H] = 1171.1 |
| FB95 | C₆₃H₈₁F₃N₁₀O₈ | [M + H] = 1163.63 | [M + H] = 1163.9 |
| FB96 | C₆₁H₈₄F₃N₁₁O₉ | [M + H] = 1172.65 | [M + H] = 1172.9 |
| FB100 | C₅₉H₇₉F₃N₁₂O₈S | [M + H] = 1173.59 | [M + H] = 1173.9 |
| FB101 | C₆₂H₈₂F₃N₁₁O₈ | [M + H] = 1166.64 | [M + H] = 1166.9 |
| FB106 | C₆₀H₈₁F₃N₁₂O₇S | [M + H] = 1171.61 | [M + H] = 1171.7 |
| FB113 | C₅₇H₇₄F₃N₁₁O₈S | [M + H] = 1130.55 | [M + H] = 1130.5 |
| FB114 | C₆₀H₇₇F₃N₁₀O₈ | [M + H] = 1123.60 | [M + H] = 1124.1 |
| FB115 | C₅₈H₈₀F₃N₁₁O₉ | [M + H] = 1132.62 | [M + H] = 1132.7 |
| FB116 | C₅₈H₇₅F₃N₁₀O₇S | [M + H] = 1113.56 | [M + H] = 1113.5 |
| FB117 | C₅₇H₇₃F₃N₁₀O₈S | [M + H] = 1115.54 | [M + H] = 1115.5 |
| FB118 | C₅₅H₇₁F₃N₁₀O₇S | [M + H] = 1073.53 | [M + H] = 1073.5 |
| FB119 | C₅₄H₆₉F₃N₁₀O₈S | [M + H] = 1075.51 | [M + H] = 1075.6 |
| FC1 | C₆₀H₇₈N₈O₉ | [M + H] = 1055.60 | [M + H] = 1055.5 |
| FC2 | C₆₁H₇₈N₈O₉ | [M + H] = 1067.60 | [M + H] = 1067.6 |
| FC3 | C₅₄H₇₁N₈O₁₁P | [M + H] = 1039.51 | [M + H] = 1039.5 |
| FC4 | C₆₀H₈₄N₈O₈Si | [M + H] = 1073.63 | [M + H] = 1073.6 |
| FC5 | C₆₀H₈₄N₈O₈Si | [M + H] = 1073.63 | [M + H] = 1073.5 |
| FC6 | C₅₉H₇₈N₈O₉ | [M + H] = 1043.60 | [M + H] = 1043.6 |
| FC7 | C₅₆H₇₂N₈O₁₀ | [M + H] = 1017.55 | [M + H] = 1017.5 |
| FC8 | C₅₈H₇₆N₈O₉ | [M + H] = 1029.58 | [M + H] = 1029.6 |
| FC9 | C₅₆H₇₂N₈O₉ | [M + H] = 1001.55 | [M + H] = 1001.5 |
| FD1 | C₅₁H₆₇N₉O₇S | [M + H] = 950.50 | [M + H] = 950.5 |
| FD2 | C₅₁H₆₇N₉O₇S | [M + H] = 950.50 | [M + H] = 950.5 |
| FD3 | C₅₇H₇₄N₈O₇ | [M + H] = 983.58 | [M + H] = 983.5 |
| FD4 | C₅₇H₇₄N₈O₇ | [M + H] = 983.58 | [M + H] = 983.9 |
| FF1 | C₅₆H₇₇N₁₁O₇S | [M + H] = 1048.58 | [M + H] = 1048.6 |
| FF2 | C₅₆H₇₄F₃N₁₁O₇S | [M + H] = 1102.55 | [M + H] = 1102.6 |
| FF3 | C₅₉H₈₂F₃N₁₁O₇ | [M + H] = 1114.65 | [M + H] = 1114.7 |
| FF4 | C₅₈H₈₀F₃N₁₁O₆ | [M + H] = 1084.63 | [M + H] = 1084.7 |
| FF5 | C₅₇H₈₀F₃N₁₁O₇ | [M + H] = 1088.63 | [M + H] = 1088.7 |
| FF6 | C₅₉H₈₀N₁₀O₆ | [M + H] = 1025.64 | [M + H] = 1025.7 |
| FF7 | C₆₁H₈₂N₁₀O₆ | [M + H] = 1051.65 | [M + H] = 1051.9 |
| FF8 | C₅₆H₇₇N₁₁O₆S | [M + H] = 1032.59 | [M + H] = 1032.7 |
| FF9 | C₅₈H₇₉N₁₁O₆S | [M + H] = 1058.60 | [M + H] = 1058.7 |
| FF10 | C₅₉H₈₅N₁₁O₇ | [M + H] = 1060.67 | [M + H] = 1060.7 |
| FF11 | C₅₆H₇₄F₃N₁₁O₆S | [M + H] = 1086.56 | [M + H] = 1086.9 |
| FF12 | C₅₉H₈₁N₁₁O₆S | [M + H] = 1072.62 | [M + H] = 1072.7 |
| FF13 | C₆₀H₈₇N₁₁O₇ | [M + H] = 1074.69 | [M + H] = 1074.7 |
| FF14 | C₆₀H₈₂F₃N₁₁O₆ | [M + H] = 1110.65 | [M + H] = 1111.1 |
| FF15 | C₆₁H₇₉F₃N₁₀O₆ | [M + H] = 1105.62 | [M + H] = 1105.7 |
| FF16 | C₅₈H₇₆F₃N₁₁O₆S | [M + H] = 1112.58 | [M + H] = 1112.6 |

### Example 15. Biological Assays of Compounds of Tables F1, F2, F3, F4, and F6

All but four compounds of Table F1, F2, F3, F4, and F6 (FA5, FA17, FA18 and FC18) exhibited an IC₅₀ of 8 μM or less in the pERK potency assay described below with respect to AsPC-1 (K-Ras G12D). All compounds herein exhibited an IC₅₀ of 7 μM or less in the cell viability assay described below with respect to AsPC-1 (K-Ras G12D).

Potency Assay: pERK

The purpose of this assay was to measure the ability of test compounds to inhibit K-Ras in cells. Activated K-Ras induces increased phosphorylation of ERK at Threonine 202 and Tyrosine 204 (pERK). This procedure measures a decrease in cellular pERK in response to test compounds. The procedure described below in NCI-H358 cells is applicable to K-Ras G12C.

Note: This protocol may be executed substituting other cell lines to characterize inhibitors of other RAS variants, including, for example, AsPC-1 (K-Ras G12D), Capan-1 (K-Ras G12V), or NCI-H1355 (K-Ras G13C).

NCI-H358 cells were grown and maintained using media and procedures recommended by the ATCC. On the day prior to compound addition, cells were plated in 384-well cell culture plates (40 μl/well) and grown overnight in a 37° C., 5% CO₂ incubator. Test compounds were prepared in 10, 3-fold dilutions in DMSO, with a high concentration of 10 mM. On the day of assay, 40 nL of test compound was added to each well of cell culture plate using an Echo550 liquid handler (LabCyte®). Concentrations of test compound were tested in duplicate. After compound addition, cells were incubated 4 hours at 37° C., 5% CO₂. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μL lysis buffer, with shaking at 600 RPM at room temperature. Lysate (10 μL) was transferred to a 384-well Opti-plate (PerkinElmer) and 5 μL acceptor mix was added. After a 2-hour incubation in the dark, 5 μL donor mix was added, the plate was sealed, and incubated 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 μL tris buffered saline (TBS) and fixed for 15 minutes with 150 μL 4% formaldehyde in TBS. Fixed cells were washed 4 times for 5 minutes with TBS containing 0.1% Triton X-100 (TBST) and then blocked with 100 μL Odyssey blocking buffer (LI-COR) for 60 minutes at room temperature. Primary antibody (pERK, CST-4370, Cell Signaling Technology) was diluted 1:200 in blocking buffer, and 50 μL were added to each well and incubated overnight at 4° C. Cells were washed 4 times for 5 minutes with TBST. Secondary antibody (IR-800CW rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a Li-COR Odyssey CLx Imager. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

Regarding G13C, another pERK assay protocol is as follows.

Note: This protocol may be executed substituting other cell lines to characterize inhibitors of other RAS variants, including, for example, AsPC-1 (K-Ras G12D), Capan-1 (K-Ras G12V), or NCI-H358 (K-Ras G12C).

MIA PaCa-2 KRAS G13C A12 cells were grown and maintained using media and procedures recommended by the ATCC. On the day prior to compound addition, cells were plated in 384-well cell culture plates (8,000 cells/40 μl/well) and grown overnight in a 37° C., 5% CO₂ incubator. Test compounds were prepared in 10, 3-fold dilutions in DMSO, with a high concentration of 10, 1 or 0.1 mM. On the day of assay, 40 nL of test compound were added to each well of cell culture plate using an Echo550 liquid handler (LabCyte®). Concentrations of test compound were tested in duplicate. After compound addition, cells were incubated 4 hours at 37° C., 5% CO₂. Following incubation, culture medium was removed and cells were washed once with phosphate buffered saline.

In some experiments, cellular pERK level was determined using the AlphaLISA SureFire Ultra p-ERK1/2 Assay Kit (PerkinElmer). Cells were lysed in 25 μL lysis buffer, with shaking at 600 RPM at room temperature. Lysate (10 μL) was transferred to a 384-well Opti-plate (PerkinElmer) and 5 μL acceptor mix was added. After a 2-hour incubation in the dark, 5 μL donor mix was added, the plate was sealed, and incubated 2 hours at room temperature. Signal was read on an Envision plate reader (PerkinElmer) using standard AlphaLISA settings. Analysis of raw data was carried out in Genedata Screener and Prism (GraphPad). Data were normalized by the following calculation: ((sample signal−average low control)/(average DMSO−average low control))*100. Signal was plotted vs. the decadal logarithm of compound concentration, and IC₅₀ was determined by fitting a 4-parameter sigmoidal concentration response model.

In other experiments, cellular pERK was determined by In-Cell Western. Following compound treatment, cells were washed twice with 200 μL tris buffered saline (TBS) and fixed for 15 minutes with 150 μL 4% formaldehyde in TBS. Fixed cells were washed 4 times for 5 minutes with TBS containing 0.1% Triton X-100 (TBST) and then blocked with 100 μL Odyssey blocking buffer (LI-COR) for 60 minutes at room temperature. Primary antibody (pERK, CST-4370, Cell Signaling Technology) was diluted 1:200 in blocking buffer, and 50 μL were added to each well and incubated overnight at 4° C. Cells were washed 4 times for 5 minutes with TBST. Secondary antibody (IR-800CW rabbit, LI-COR, diluted 1:800) and DNA stain DRAQ5 (LI-COR, diluted 1:2000) were added and incubated 1-2 hours at room temperature. Cells were washed 4 times for 5 minutes with TBST. Plates were scanned on a Li-COR Odyssey CLx Imager. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

### Determination of Cell Viability in RAS Mutant Cancer Cell Lines

### Protocol: CellTiter-Glo® Cell Viability Assay

Note—The following protocol describes a procedure for monitoring cell viability of K-Ras mutant cancer cell lines in response to a compound of the invention. Other RAS isoforms may be employed, though the number of cells to be seeded will vary based on cell line used.

The purpose of this cellular assay was to determine the effects of test compounds on the proliferation of three human cancer cell lines (NCI-H358 (K-Ras G12C), AsPC-1 (K-Ras G12D), and Capan-1 (K-Ras G12V)) over a 5-day treatment period by quantifying the amount of ATP present at endpoint using the CellTiter-Glo® 2.0 Reagent (Promega).

Cells were seeded at 250 cells/well in 40 μL of growth medium in 384-well assay plates and incubated overnight in a humidified atmosphere of 5% CO₂ at 37° C. On the day of the assay, 10 mM stock solutions of test compounds were first diluted into 3 mM solutions with 100% DMSO. Well-mixed compound solutions (15 μL) were transferred to the next wells containing 30 μL of 100% DMSO, and repeated until a 9-concentration 3-fold serial dilution was made (starting assay concentration of 10 μM). Test compounds (132.5 nL) were directly dispensed into the assay plates containing cells. The plates were shaken for 15 seconds at 300 rpm, centrifuged, and incubated in a humidified atmosphere of 5% CO₂ at 37° C. for 5 days. On day 5, assay plates and their contents were equilibrated to room temperature for approximately 30 minutes. CellTiter-Glo® 2.0 Reagent (25 μL) was added, and plate contents were mixed for 2 minutes on an orbital shaker before incubation at room temperature for 10 minutes. Luminescence was measured using the PerkinElmer Enspire. Data were normalized by the following: (Sample signal/Avg. DMSO)*100. The data were fit using a four-parameter logistic fit.

Another CTG assay protocol employed with respect to MIA PaCa-2 KRAS G13C A12 (K-Ras G13C, in particular, is as follows, Note: other RAS isoforms may be employed (e.g., NCI-H358 (K-Ras G12C), AsPC-1 (K-Ras G12D), and Capan-1 (K-Ras G12V)), though the number of cells to be seeded will vary based on cell line used).

The purpose of this cellular assay was to determine the effects of test compounds on the proliferation of human cancer cell lines over a 5-day treatment period by quantifying the amount of ATP present at endpoint using the CellTiter-Glo® 2.0 Reagent (Promega).

Cells were seeded at 250 cells/well in 40 μL of growth medium in 384-well assay plates and incubated overnight in a humidified atmosphere of 5% CO₂ at 37° C. Test compounds were prepared in 9 point, 3-fold dilutions in DMSO, with a high concentration of 10, 1 or 0.1 mM. On the day of the assay, test compounds (40 nL) were directly dispensed into the assay plates containing cells. The plates were shaken for 15 seconds at 300 rpm, centrifuged, and incubated in a humidified atmosphere of 5% CO₂ at 37° C. for 5 days. On day 5, assay plates and their contents were equilibrated to room temperature for approximately 30 minutes. CellTiter-Glo® 2.0 Reagent (25 μL) was added, and plate contents were mixed for 2 minutes on an orbital shaker before incubation at room temperature for 10 minutes. Luminescence was measured using the PerkinElmer Enspire. Data were normalized by the following: (Sample signal/Avg. DMSO)*100. The data were fit using a four-parameter logistic fit.

Disruption of B-Raf Ras-Binding Domain (BRAF^{RBD}) Interaction with K-Ras by Compounds of the Invention (Also Called a FRET Assay or an MOA Assay)

Note—The following protocol describes a procedure for monitoring disruption of K-Ras G12C (GMP-PNP) binding to BRAF^{RBD} by a compound of the invention. This protocol may also be executed substituting other Ras proteins or nucleotides, such as K-Ras G12D and K-Ras G13D.

The purpose of this biochemical assay was to measure the ability of test compounds to facilitate ternary complex formation between a nucleotide-loaded K-Ras isoform and Cyclophilin A; the resulting ternary complex disrupts binding to a BRAF^{RBD} construct, inhibiting K-Ras signaling through a RAF effector. Data was reported as IC₅₀ values.

In assay buffer containing 25 mM HEPES pH 7.3, 0.002% Tween20, 0.1% BSA, 100 mM NaCl and 5 mM MgCl₂, tagless Cyclophilin A, His 6-K-Ras-GMPPNP, and GST-BRAF^{RBD} were combined in a 384-well assay plate at final concentrations of 25 μM, 12.5 nM, and 50 nM, respectively. Compound was present in plate wells as a 10-point 3-fold dilution series starting at a final concentration of 30 μM. After incubation at 25° C. for 3 hours, a mixture of anti-His Eu-W1024 and anti-GST allophycocyanin was then added to assay sample wells at final concentrations of 10 nM and 50 nM, respectively, and the reaction incubated for an additional 1.5 hours. TR-FRET signal was read on a microplate reader (Ex 320 nm, Em 665/615 nm). Compounds that facilitate disruption of a K-Ras:RAF complex were identified as those eliciting a decrease in the TR-FRET ratio relative to DMSO control wells.

Cross-Linking of Ras Proteins with Compounds of the Invention to Form Conjugates

The following cross-linking assay describes a method of determining covalent adduct formation by a compound of the present invention with a Ras protein.

Note—The following protocol describes a procedure for monitoring cross-linking of K-Ras G12C (GMP-PNP) to a compound of the invention. This protocol may also be executed substituting other Ras proteins or nucleotides, such as such as K-Ras G12D and K-Ras G13D.

The purpose of this biochemical assay was to measure the ability of test compounds to covalently label nucleotide-loaded K-Ras isoforms. In assay buffer containing 12.5 mM HEPES pH 7.4, 75 mM NaCl, 1 mM MgCl₂, 1 mM BME, 5 μM Cyclophilin A, and 2 μM test compound, a 5 μM stock of GMP-PNP-loaded K-Ras (1-169) G12C was diluted 10-fold to yield a final concentration of 0.5 μM; with final sample volume being 100 μL.

The sample was incubated at 25° C. for a time period of up to 24 hours prior to quenching by the addition of 10 μL of 5% Formic Acid. Quenched samples were centrifuged at 15000 rpm for 15 minutes in a benchtop centrifuge before injecting a 10 μL aliquot onto a reverse phase C₄ column and eluting into the mass spectrometer with an increasing acetonitrile gradient in the mobile phase. Analysis of raw data was carried out using Waters MassLynx MS software, with % bound calculated from the deconvoluted protein peaks for labeled and unlabeled K-Ras.

### In Vitro Cell Proliferation Panels

Potency for inhibition of cell growth may be assessed at CrownBio using standard methods. Briefly, cell lines are cultured in appropriate medium, and then plated in 3D methylcellulose. Inhibition of cell growth is determined by CellTiter-Glo® after 5 days of culture with increasing concentrations of compounds. Compound potency is reported as the 50% inhibition concentration (absolute IC₅₀). The assay took place over 7 days. On day 1, cells in 2D culture are harvested during logarithmic growth and suspended in culture medium at 1×105 cells/ml. Higher or lower cell densities are used for some cell lines based on prior optimization. 3.5 ml of cell suspension is mixed with 6.5% growth medium with 1% methylcellulose, resulting in a cell suspension in 0.65% methylcellulose. 90 μl of this suspension is distributed in the wells of 2 96-well plates. One plate is used for day 0 reading and 1 plate is used for the end-point experiment. Plates are incubated overnight at 37 C with 5% CO₂. On day 2, one plate (for t0 reading) is removed and 10 μl growth medium plus 100 μl CellTiter-Glo® Reagent is added to each well. After mixing and a 10-minute incubation, luminescence is recorded on an EnVision Multi-Label Reader (Perkin Elmer). Compounds in DMSO are diluted in growth medium such that the final, maximum concentration of compound is 10 μM, and serial 4-fold dilutions are performed to generate a 9-point concentration series. 10 μl of compound solution at 10 times final concentration is added to wells of the second plate. Plate is then incubated for 120 hours at 37C and 5% CO₂. On day 7 the plates are removed, 100 μl CellTiter-Glo® Reagent is added to each well, and after mixing and a 10-minute incubation, luminescence is recorded on an EnVision Multi-Label Reader (Perkin Elmer). Data is exported to GeneData Screener and modeled with a sigmoidal concentration response model in order to determine the IC₅₀ for compound response.

Not all cell lines with a given RAS mutation may be equally sensitive to a RAS inhibitor targeting that mutation, due to differential expression of efflux transporters, varying dependencies on RAS pathway activation for growth, or other reasons. This has been exemplified by the cell line KYSE-410 which, despite having a KRAS G12C mutation, is insensitive to the KRAS G12C (OFF) inhibitor MRTX-849 (Hallin et al., Cancer Discovery 10:54-71 (2020)), and the cell line SW1573, which is insensitive to the KRAS G12C (OFF) inhibitor AMG510 (Canon et al., Nature 575:217-223 (2019)).

### OTHER EMBODIMENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims. Other embodiments are within the claims.

1. A method of treating cancer in a subject in need thereof, wherein the cancer comprises:
(a) a first RAS mutation that is G12C and a second RAS mutation at a position selected from the group consisting of H95, R68, G13 and Q61, or a second RAS mutation that is selected from the group consisting of Y96C, Y96F, Y96H, Y96N, Y96S; or
(b) a first RAS mutation at position G12 selected from the group consisting of G12H, G12I, G12K, G12M, G12N, G12P, G12Q, G12T, G12W and G12Y,
wherein the cancer is resistant to treatment with a RAS(OFF) inhibitor, the method comprising administering to the subject a RAS(ON) inhibitor.

2. The method of claim 1, wherein the cancer comprises a first RAS mutation that is G12C and a second RAS mutation at position H95 or R68.

3. The method of claim 2, wherein the second RAS mutation is selected from the group consisting of H95D, H95L, H95N, H95P, H95Q, H95R and H95Y.

4. The method of claim 2, wherein the second mutation is selected from the group consisting of R68G, R68K, R68M, R68S, R68T and R68W.

5. The method of claim 1, wherein any RAS mutation is selected from a KRAS mutation, an NRAS mutation, and an HRAS mutation.

6. The method of claim 1, wherein the RAS(ON) inhibitor is an inhibitor selective for RAS G12C, G13D, or G12D.

7. The method of claim 1, wherein the RAS(ON) inhibitor is a RAS(ON)^{MULTI} inhibitor.

8. The method of claim 1, wherein the RAS(ON) inhibitor is a tri-complex RAS(ON) inhibitor.

9. The method of claim 1, wherein the RAS(ON) inhibitor is a compound of Formula AI: 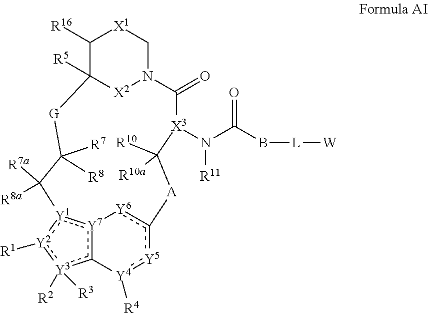
or a pharmaceutically acceptable salt thereof,
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, cyano, S(O)₂R′, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₁-C₄ alkyl optionally substituted 3 to 11-membered heterocycloalkyl, optionally substituted 3 to 8-membered cycloalkyl, or optionally substituted 3 to 8-membered heteroaryl;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl;
R¹⁶ is hydrogen or C₁-C₃ alkyl.

10. The method of claim 1, wherein the RAS(ON) inhibitor is a compound of Formula BI: 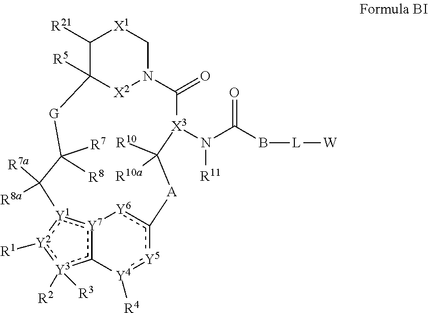
or a pharmaceutically acceptable salt thereof,
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is absent, —CH(R⁹)—, >C═CR⁹R⁹′, or >CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a vinyl ketone, a vinyl sulfone, an ynone, a haloacetyl, or an alkynyl sulfone;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is H, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl; or
R⁹ and R⁹′, combined with the atoms to which they are attached, form a 3 to 6-membered cycloalkyl or a 3 to 6-membered heterocycloalkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R²¹ is hydrogen or C₁-C₃ alkyl.

11. The method of claim 1, wherein the RAS(ON) inhibitor is a compound of Formula CI: 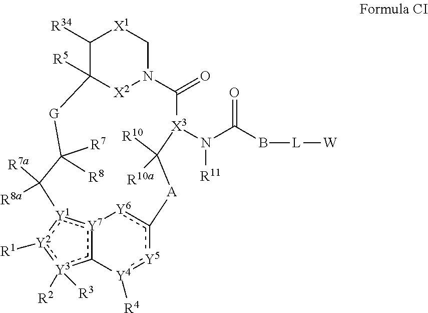
or a pharmaceutically acceptable salt thereof,
wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted 3 to 6-membered cycloalkylene, optionally substituted 3 to 6-membered heterocycloalkylene, optionally substituted 6-membered arylene, or optionally substituted 5 to 10-membered heteroarylene;
B is —CH(R⁹)— or >C═CR⁹R⁹′ where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is a cross-linking group comprising a carbodiimide, an oxazoline, a thiazoline, a chloroethyl urea, a chloroethyl thiourea, a chloroethyl carbamate, a chloroethyl thiocarbamate, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R⁸′; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷′ is hydrogen, halogen, or optionally substituted C₁-C₃ alkyl; R⁸′ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷′ and R⁸′ combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl, or
R⁹ and L combine with the atoms to which they are attached to form an optionally substituted 3 to 14-membered heterocycloalkyl;
R⁹′ is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo; and
R¹¹ is hydrogen or C₁-C₃ alkyl; and
R³⁴ is hydrogen or C₁-C₃ alkyl.

12. The method of claim 1, wherein the RAS(ON) inhibitor is a compound of Formula DIa: 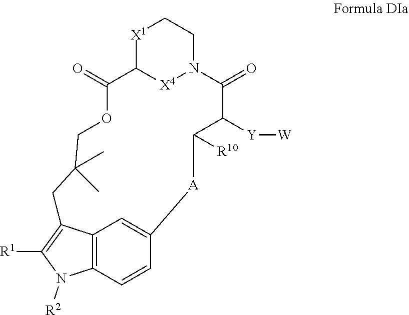
or a pharmaceutically acceptable salt thereof,
wherein A is optionally substituted 3 to 6-membered cycloalkylene, optionally substituted 3 to 6-membered heterocycloalkylene, optionally substituted 6-membered arylene, optionally substituted 5 to 6-membered heteroarylene, optionally substituted C₂-C₄ alkylene, or optionally substituted C₂-C₄ alkenylene;
Y is 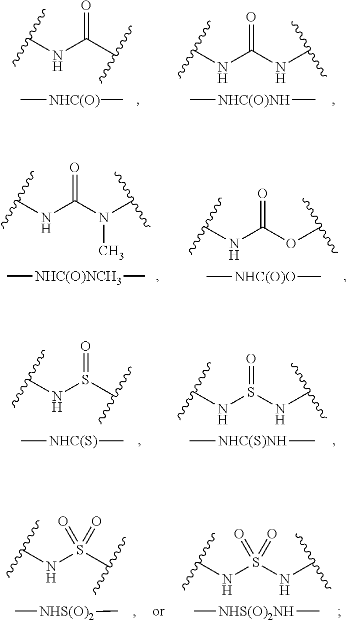
W is hydrogen, C₁-C₄ alkyl, optionally substituted C₁-C₃ heteroalkyl, optionally substituted 3 to 10-membered heterocycloalkyl, optionally substituted 3 to 10-membered cycloalkyl, optionally substituted 6 to 10-membered aryl, or optionally substituted 5 to 10-membered heteroaryl;
X¹ and X⁴ are each, independently, CH₂ or NH;
R¹ is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 15-membered heterocycloalkyl, optionally substituted 6 to 10-membered aryl, or optionally substituted 5 to 10-membered heteroaryl; and
R² is hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; and R¹⁰ is hydrogen, hydroxy, optionally substituted C₁-C₃ alkyl, or optionally substituted C₁-C₆ heteroalkyl.

13. The method of claim 1, wherein the RAS(ON) inhibitor is a compound of Formula EI: 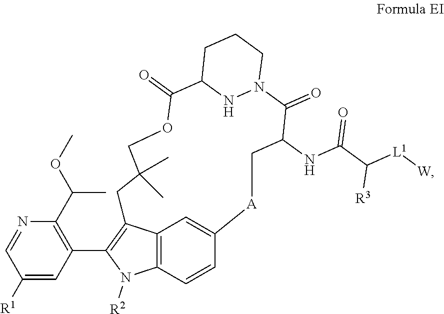
or a pharmaceutically acceptable salt thereof,
wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
L¹ is absent or a linker;
W is a cross-linking group comprising a vinyl ketone, vinyl sulfone, ynone, or an alkynyl sulfone:
R¹ is hydrogen, optionally substituted 3 to 10-membered heterocycloalkyl, or optionally substituted C₁-C₆ heteroalkyl;
R² is optionally substituted C₁-C₆ alkyl; and
R³ is optionally substituted C₁-C₆ alkyl or optionally substituted C₁-C₃ heteroalkyl.

14. The method of claim 1, wherein the RAS(ON) inhibitor is a compound of Formula FI: 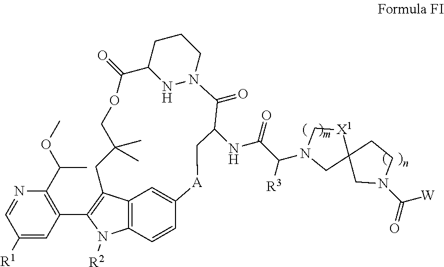
or a pharmaceutically acceptable salt thereof,
wherein A is optionally substituted 3 to 6-membered heterocycloalkylene, optionally substituted 3 to 6-membered cycloalkylene, optionally substituted 6-membered arylene, or optionally substituted 5 to 10-membered heteroarylene;
W is a cross-linking group comprising an aziridine, an epoxide, a carbodiimide, an oxazoline, a thiazoline, a chloroethyl urea, a chloroethyl thiourea, a chloroethyl carbamate, a chloroethyl thiocarbamate, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an oxazolium, or a glycal;
X¹ is CH₂ or O;
m is 1 or 2;
n is 0 or 1;
R¹ is hydrogen or optionally substituted 3 to 10-membered heterocycloalkyl;
R² is optionally substituted C₁-C₆ alkyl; and
R³ is optionally substituted C₁-C₆ alkyl or optionally substituted 3 to 6-membered cycloalkyl.
